(19) United States
(12) Patent Application Publication
(10) Pub. No.: US 2004/0033493 A1
(43) Pub. Date: Feb. 19, 2004

(54) Proteins and nucleic acids encoding same

(76) Inventors: Velizar T. Tchernev, Branford, CT (US); Kimberly A. Spytek, New Haven, CT (US); Bryan D. Zerhusen, Branford, CT (US); Meera Patturajan, Branford, CT (US); Richard A. Shimkets, West Haven, CT (US); Li Li, Branford, CT (US); Esha A. Gangolli, Madison, CT (US); Muralidhara Padigaru, Branford, CT (US); David W. Anderson, Branford, CT (US); Luca Rastelli, Guilford, CT (US); Charles E. Miller, Hill Drive, CT (US); Valerie Gerlach, Branford, CT (US); Raymond J. Taupier JR., East Haven, CT (US); Vladimir Y. Gusev, (US); Steven D. Colman, Guilford, CT (US); Adam Ryan Wolenc, New Haven, CT (US); Carol E. A. Pena, Guilford, CT (US); Katarzyna Furtak, Anosia, CT (US); William M. Grosse, Bransford, CT (US); John P. Alsobrook II, Madison, CT (US); Denise M. Lepley, Branford, CT (US); Daniel K. Rieger, Branford, CT (US); Catherine E. Burgess, Wethersfield, CT (US)

Correspondence Address: Ivor R. Elrifi, Ph.D., Mintz, Levin, Cohn, Ferris,, Glovsky and Popeo, P.C., One Financial Center, Boston, MA, 02111, US

(21) Appl. No.: 10/072,012

(22) Filed: Jan. 31, 2002

Related U.S. Application Data
(60) Provisional application No. 60/267,459, filed on Feb. 8, 2001.
(60) Provisional application No. 60/266,975, filed on Feb. 7, 2001.
(60) Provisional application No. 60/267,057, filed on Feb. 7, 2001.
(60) Provisional application No. 60/266,767, filed on Feb. 5, 2001.
(60) Provisional application No. 60/266,406, filed on Feb. 2, 2001.
(60) Provisional application No. 60/265,395, filed on Jan. 31, 2001.
(60) Provisional application No. 60/265,412, filed on Jan. 31, 2001.
(60) Provisional application No. 60/265,517, filed on Jan. 31, 2001.
(60) Provisional application No. 60/265,514, filed on Jan. 31, 2001.
(60) Provisional application No. 60/267,823, filed on Feb. 9, 2001.
(60) Provisional application No. 60/268,974, filed on Feb. 15, 2001.
(60) Provisional application No. 60/271,855, filed on Feb. 27, 2001.
(60) Provisional application No. 60/271,839, filed on Feb. 27, 2001.
(60) Provisional application No. 60/273,046, filed on Mar. 2, 2001.
(60) Provisional application No. 60/272,788, filed on Mar. 2, 2001.
(60) Provisional application No. 60/275,989, filed on Mar. 14, 2001.
(60) Provisional application No. 60/275,925, filed on Mar. 14, 2001.
(60) Provisional application No. 60/275,947, filed on Mar. 14, 2001.
(60) Provisional application No. 60/275,950, filed on Mar. 14, 2001.
(60) Provisional application No. 60/276,450, filed on Mar. 15, 2001.
(60) Provisional application No. 60/276,448, filed on Mar. 15, 2001.
(60) Provisional application No. 60/276,397, filed on Mar. 16, 2001.
(60) Provisional application No. 60/276,768, filed on Mar. 16, 2001.
(60) Provisional application No. 60/278,652, filed on Mar. 20, 2001.
(60) Provisional application No. 60/278,775, filed on Mar. 26, 2001.
(60) Provisional application No. 60/278,778, filed on Mar. 26, 2001.
(60) Provisional application No. 60/279,882, filed on Mar. 29, 2001.
(60) Provisional application No. 60/279,884, filed on Mar. 29, 2001.
(60) Provisional application No. 60/280,147, filed on Mar. 30, 2001.
(60) Provisional application No. 60/283,083, filed on Apr. 11, 2001.
(60) Provisional application No. 60/282,992, filed on Apr. 11, 2001.
(60) Provisional application No. 60/285,133, filed on Apr. 20, 2001.
(60) Provisional application No. 60/285,749, filed on Apr. 23, 2001.
(60) Provisional application No. 60/288,327, filed on May 3, 2001.
(60) Provisional application No. 60/288,504, filed on May 3, 2001.
(60) Provisional application No. 60/294,047, filed on May 29, 2001.
(60) Provisional application No. 60/294,473, filed on May 30, 2001.
(60) Provisional application No. 60/296,964, filed on Jun. 8, 2001.
(60) Provisional application No. 60/298,959, filed on Jun. 18, 2001.
(60) Provisional application No. 60/299,324, filed on Jun. 19, 2001.
(60) Provisional application No. 60/312,020, filed on Aug. 13, 2001.
(60) Provisional application No. 60/312,908, filed on Aug. 16, 2001.
(60) Provisional application No. 60/312,889, filed on Aug. 16, 2001.
(60) Provisional application No. 60/313,930, filed on Aug. 21, 2001.
(60) Provisional application No. 60/315,470, filed on Aug. 28, 2001.
(60) Provisional application No. 60/316,447, filed on Aug. 31, 2001.
(60) Provisional application No. 60/318,115, filed on Sep. 7, 2001.
(60) Provisional application No. 60/318,118, filed on Sep. 7, 2001.
(60) Provisional application No. 60/318,740, filed on Sep. 12, 2001.
(60) Provisional application No. 60/323,379, filed on Sep. 19, 2001.
(60) Provisional application No. 60/330,308, filed on Oct. 18, 2001.
(60) Provisional application No. 60/330,245, filed on Oct. 18, 2001.
(60) Provisional application No. 60/332,701, filed on Nov. 14, 2001.
(60) Provisional application No. 60/271,664, filed on Feb. 26, 2001.

(51) Int. Cl. C12Q 1/68; G01N 33/574; C07H 21/04; A61K 39/395; C12N 9/00; C12P 21/02; C12N 5/06; C07K 14/47

(52) U.S. Cl. 435/6; 435/7.23; 435/69.3; 435/320.1; 435/325; 530/350; 536/23.2; 435/183; 424/155.1

(57) ABSTRACT

Disclosed herein are nucleic acid sequences that encode novel polypeptides. Also disclosed are polypeptides encoded by these nucleic acid sequences, and antibodies, which immunospecifically-bind to the polypeptide, as well as derivatives, variants, mutants, or fragments of the aforementioned polypeptide, polynucleotide, or antibody. The invention further discloses therapeutic, diagnostic and research methods for diagnosis, treatment, and prevention of disorders involving any one of these novel human nucleic acids and proteins.

### FIELD OF THE INVENTION

The invention generally relates to nucleic acids and polypeptides encoded thereby.

### BACKGROUND OF THE INVENTION

The invention generally relates to nucleic acids and polypeptides encoded therefrom. More specifically, the invention relates to nucleic acids encoding cytoplasmic, nuclear, membrane bound, and secreted polypeptides, as well as vectors, host cells, antibodies, and recombinant methods for producing these nucleic acids and polypeptides.

### SUMMARY OF THE INVENTION

The invention is based in part upon the discovery of nucleic acid sequences encoding novel polypeptides. The novel nucleic acids and polypeptides are referred to herein as NOVX, or NOV1-99 nucleic acids and polypeptides. These nucleic acids and polypeptides, as well as derivatives, homologs, analogs and fragments thereof, will hereinafter be collectively designated as “NOVX” nucleic acid or polypeptide sequences.

In one aspect, the invention provides an isolated NOVX nucleic acid molecule encoding a NOVX polypeptide that includes a nucleic acid sequence that has identity to the nucleic acids disclosed in SEQ ID NOS:2n−1, wherein n is an integer between 1 and 162,. In some embodiments, the NOVX nucleic acid molecule will hybridize under stringent conditions to a nucleic acid sequence complementary to a nucleic acid molecule that includes a protein-coding sequence of a NOVX nucleic acid sequence. The invention also includes an isolated nucleic acid that encodes a NOVX polypeptide, or a fragment, homolog, analog or derivative thereof. For example, the nucleic acid can encode a polypeptide at least 80% identical to a polypeptide comprising the amino acid sequences of SEQ ID NOS:2n, wherein n is an integer between 1 and 162. The nucleic acid can be, for example, a genomic DNA fragment or a cDNA molecule that includes the nucleic acid sequence of any of SEQ ID NOS:2n−1, wherein n is an integer between 1 and 162.

Also included in the invention is an oligonucleotide, e.g., an oligonucleotide which includes at least 6 contiguous nucleotides of a NOVX nucleic acid (e.g., SEQ ID NOS:2n−1, wherein n is an integer between 1 and 162) or a complement of said oligonucleotide.

Also included in the invention are substantially purified NOVX polypeptides (SEQ ID NOS:2n, wherein n is an integer between 1 and 162). In certain embodiments, the NOVX polypeptides include an amino acid sequence that is substantially identical to the amino acid sequence of a human NOVX polypeptide.

The invention also features antibodies that immunoselectively bind to NOVX polypeptides, or fragments, homologs, analogs or derivatives thereof.

In another aspect, the invention includes pharmaceutical compositions that include therapeutically- or prophylactically-effective amounts of a therapeutic and a pharmaceutically-acceptable carrier. The therapeutic can be, e.g., a NOVX nucleic acid, a NOVX polypeptide, or an antibody specific for a NOVX polypeptide. In a further aspect, the invention includes, in one or more containers, a therapeutically- or prophylactically-effective amount of this pharmaceutical composition.

In a further aspect, the invention includes a method of producing a polypeptide by culturing a cell that includes a NOVX nucleic acid, under conditions allowing for expression of the NOVX polypeptide encoded by the DNA. If desired, the NOVX polypeptide can then be recovered.

In another aspect, the invention includes a method of detecting the presence of a NOVX polypeptide in a sample. In the method, a sample is contacted with a compound that selectively binds to the polypeptide under conditions allowing for formation of a complex between the polypeptide and the compound. The complex is detected, if present, thereby identifying the NOVX polypeptide within the sample.

The invention also includes methods to identify specific cell or tissue types based on their expression of a NOVX.

Also included in the invention is a method of detecting the presence of a NOVX nucleic acid molecule in a sample by contacting the sample with a NOVX nucleic acid probe or primer, and detecting whether the nucleic acid probe or primer bound to a NOVX nucleic acid molecule in the sample.

In a further aspect, the invention provides a method for modulating the activity of a NOVX polypeptide by contacting a cell sample that includes the NOVX polypeptide with a compound that binds to the NOVX polypeptide in an amount sufficient to modulate the activity of said polypeptide. The compound can be, e.g., a small molecule, such as a nucleic acid, peptide, polypeptide, peptidomimetic, carbohydrate, lipid or other organic (carbon containing) or inorganic molecule, as further described herein.

Also within the scope of the invention is the use of a therapeutic in the manufacture of a medicament for treating or preventing various disorders or syndromes described below.

The therapeutic can be, e.g., a NOVX nucleic acid, a NOVX polypeptide, or a NOVX-specific antibody, or biologically-active derivatives or fragments thereof.

For example, the compositions of the present invention will have efficacy for treatment of patients suffering from the diseases and disorders disclosed above and/or other pathologies and disorders of the like. The polypeptides can be used as immunogens to produce antibodies specific for the invention, and as vaccines. They can also be used to screen for potential agonist and antagonist compounds. For example, a cDNA encoding NOVX may be useful in gene therapy, and NOVX may be useful when administered to a subject in need thereof. By way of non-limiting example, the compositions of the present invention will have efficacy for treatment of patients suffering from the diseases and disorders disclosed above and/or other pathologies and disorders of the like.

The invention further includes a method for screening for a modulator of disorders or syndromes including, e.g., the diseases and disorders disclosed above and/or other pathologies and disorders of the like. The method includes contacting a test compound with a NOVX polypeptide and determining if the test compound binds to said NOVX polypeptide. Binding of the test compound to the NOVX polypeptide indicates the test compound is a modulator of activity, or of latency or predisposition to the aforementioned disorders or syndromes.

Also within the scope of the invention is a method for screening for a modulator of activity, or of latency or predisposition to disorders or syndromes including, e.g., the diseases and disorders disclosed above and/or other pathologies and disorders of the like by administering a test compound to a test animal at increased risk for the aforementioned disorders or syndromes. The test animal expresses a recombinant polypeptide encoded by a NOVX nucleic acid. Expression or activity of NOVX polypeptide is then measured in the test animal, as is expression or activity of the protein in a control animal which recombinantly-expresses NOVX polypeptide and is not at increased risk for the disorder or syndrome. Next, the expression of NOVX polypeptide in both the test animal and the control animal is compared. A change in the activity of NOVX polypeptide in the test animal relative to the control animal indicates the test compound is a modulator of latency of the disorder or syndrome.

In yet another aspect, the invention includes a method for determining the presence of or predisposition to a disease associated with altered levels of a NOVX polypeptide, a NOVX nucleic acid, or both, in a subject (e.g., a human subject). The method includes measuring the amount of the NOVX polypeptide in a test sample from the subject and comparing the amount of the polypeptide in the test sample to the amount of the NOVX polypeptide present in a control sample. An alteration in the level of the NOVX polypeptide in the test sample as compared to the control sample indicates the presence of or predisposition to a disease in the subject. Preferably, the predisposition includes, e.g., the diseases and disorders disclosed above and/or other pathologies and disorders of the like. Also, the expression levels of the new polypeptides of the invention can be used in a method to screen for various cancers as well as to determine the stage of cancers.

In a further aspect, the invention includes a method of treating or preventing a pathological condition associated with a disorder in a mammal by administering to the subject a NOVX polypeptide, a NOVX nucleic acid, or a NOVX-specific antibody to a subject (e.g., a human subject), in an amount sufficient to alleviate or prevent the pathological condition. In preferred embodiments, the disorder, includes, e.g., the diseases and disorders disclosed above and/or other pathologies and disorders of the like.

In yet another aspect, the invention can be used in a method to identity the cellular receptors and downstream effectors of the invention by any one of a number of techniques commonly employed in the art. These include but are not limited to the two-hybrid system, affinity purification, co-precipitation with antibodies or other specific-interacting molecules.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### DETAILED DESCRIPTION OF THE INVENTION

The present invention provides novel nucleotides and polypeptides encoded thereby. Included in the invention are the novel nucleic acid sequences and their encoded polypeptides. The sequences are collectively referred to herein as “NOVX nucleic acids” or “NOVX polynucleotides” and the corresponding encoded polypeptides are referred to as “NOVX polypeptides” or “NOVX proteins.” Unless indicated otherwise, “NOVX” is meant to refer to any of the novel sequences disclosed herein. Table A provides a summary of the NOVX nucleic acids and their encoded polypeptides.

TABLE A
Sequences and Corresponding SEQ ID Numbers

| | | SEQ ID | | |
|---|---|---|---|---|
| | | NO | | |
| NOVX | | (nucleic | SEQ ID NO | |
| Assignment | Internal Identification | acid) | (polypeptide) | Homology |
| 1a | CG56592-01 | 1 | 2 | Claudin 6-like |
| 1b | CG56586-01 | 3 | 4 | Claudin-3-like |
| 1c | CG56592-03 | 5 | 6 | Claudin-6-like |
| 1d | CG56592-02 | 7 | 8 | Claudin 6-like |
| 2 | CG56596-01 | 9 | 10 | Protein Serine Kinase-like |
| 3a | CG56594-01 | 11 | 12 | Claudin-19-like |
| 3b | CG56594-02 | 13 | 14 | Claudin-19-like |
| 3c | CG57576-01 | 15 | 16 | Claudin-19-like |
| 4a | CG56589-01 | 17 | 18 | Claudin-6-like |
| 4b | CG56589-01 | 19 | 20 | Claudin-6-like |
| 4c | CG56589-02 | 21 | 22 | Claudin-6-like |
| 5a | CG56635-01 | 23 | 24 | Monocarboxylate transporter |
| | | | | (MCT3)-like |
| 5b | CG56635-02 | 25 | 26 | Monocarboxylate transporter |
| | | | | 3-like |
| 5c | CG56635-03 | 27 | 28 | Monocarboxylate transporter |
| | | | | 3-like |
| 5d | CG56635-04 | 29 | 30 | Monocarboxylate transporter |
| | | | | 3-like |
| 5e | CG56635-05 | 31 | 32 | Monocarboxylate transporter |
| | | | | 3-like |
| 6 | CG56674-01 | 33 | 34 | Nitrilase-1-like |
| 7a | CG56613-01 | 35 | 36 | Cleavage Signal-1 Protein- |
| | | | | Like |
| 7b | CG56613-02 | 37 | 38 | Cleavage Signal-1 Protein- |
| | | | | Like |
| 7c | CG56613-03 | 39 | 40 | Cleavage Signal-1 Protein- |
| | | | | Like |
| 7d | 174307820 | 41 | 42 | Cleavage Signal-1 Protein- |
| | | | | Like |
| 7e | 167474749 | 323 | 324 | Cleavage Signal-1 Protein- |
| | | | | Like |
| 8 | 153472451 | 43 | 44 | Matriptase-like |
| 9a | CG56554-01 | 45 | 46 | Neuropeptide Y/Peptide YY |
| | | | | receptor-like |
| 9b | CG56554-02 | 47 | 48 | Neuropeptide Y/Peptide YY |
| | | | | receptor-like |
| 10 | CG55964-01 | 49 | 50 | G-Protein Coupled Receptor- |
| | | | | like |
| 11 | CG55966-01 | 51 | 52 | G-Protein Coupled Receptor- |
| | | | | like |
| 12 | CG56003-01 | 53 | 54 | Neuromodulin-like |
| 13a | CG56021-01 | 55 | 56 | G-Protein Coupled Receptor- |
| | | | | like |
| 13b | CG56021-02 | 57 | 58 | G-Protein Coupled Receptor- |
| | | | | like |
| 14 | CG56023-01 | 59 | 60 | G-Protein Coupled Receptor- |
| | | | | like |
| 15a | CG56065-01 | 61 | 62 | G-Protein Coupled Receptor- |
| | | | | like |
| 15b | CG56065-02 | 63 | 64 | G-Protein Coupled Receptor- |
| | | | | like |
| 16a | CG56067-01 | 65 | 66 | G-Protein Coupled Receptor- |
| | | | | like |
| 16b | CG56753-02 | 67 | 68 | G-Protein Coupled Receptor- |
| | | | | like |
| 17a | CG56657-01 | 69 | 70 | G-Protein Coupled Receptor- |
| | | | | like |
| 17b | CG56657-02 | 71 | 72 | G-Protein Coupled Receptor- |
| | | | | like |
| 17c | CG56659-01 | 73 | 74 | G-Protein Coupled Receptor- |
| | | | | like |
| 17d | CG56659-02 | 75 | 76 | G-Protein Coupled Receptor- |
| | | | | like |
| 18a | CG56663-01 | 77 | 78 | G-Protein Coupled Receptor- |
| | | | | like |
| 18b | CG56663-02 | 79 | 80 | G-Protein Coupled Receptor- |
| | | | | like |
| 19a | CG56665-01 | 81 | 82 | G-Protein Coupled Receptor- |
| | | | | like |
| 19b | CG56665-02 | 83 | 84 | G-Protein Coupled Receptor- |
| | | | | like |
| 20 | CG56667-01 | 85 | 86 | G-Protein Coupled Receptor- |
| | | | | like |
| 21a | CG56639-01 | 87 | 88 | Adrenal Secretory Serine |
| | | | | Protease-Like |
| 21b | CG56639-02 | 89 | 90 | Adrenal Secretory Serine |
| | | | | Protease-Like |
| 22a | CG56643-01 | 91 | 92 | Adrenal Secretory Serine |
| | | | | Protease-Like |
| 22b | CG56643-02 | 93 | 94 | Adrenal Secretory Serine |
| | | | | Protease-Like |
| 22c | CG56643-03 | 95 | 96 | Adrenal Secretory Serine |
| | | | | Protease-Like |
| 23a | CG56647-02 | 97 | 98 | Serine Protease DESC1-like |
| 23b | CG56647-03 | 99 | 100 | Serine Protease DESC1-like |
| 23c | CG56647-01 | 101 | 102 | Serine Protease DESC1-like |
| 24a | CG56155-01 | 103 | 104 | Parchorin-like |
| 24b | CG56155-02 | 105 | 106 | Parchorin-like |
| 25 | CG56457-01 | 107 | 108 | Protein Phosphatase-like |
| 26a | CG56461-01 | 109 | 110 | GAGE-7-like |
| 26b | CG56461-02 | 111 | 112 | GAGE-7-like |
| 27a | CG56645-01 | 113 | 114 | Sodium-Glucose |
| | | | | Cotransporter-like |
| 27b | CG56645-02 | 115 | 116 | Sodium-Glucose |
| | | | | Cotransporter-like |
| 27c | 191828203 | 117 | 118 | Sodium-Glucose |
| | | | | Cotransporter-like |
| 28 | CG56185-01 | 119 | 120 | MYD-1-like |
| 29a | CG56187-01 | 121 | 122 | CRAL-TRIO-like |
| 29b | CG56187-03 | 123 | 124 | CRAL-TRIO-like |
| 29c | CG56189-01 | 125 | 126 | CRAL-TRIO-like |
| 30 | CG56191-01 | 127 | 128 | Ryudocan-like |
| 31 | CG56392-01 | 129 | 130 | Sulfur-rich Keratin-like |
| 32 | CG56686-01 | 131 | 132 | DNMT1 associated protein-1 |
| | | | | (DMAP) |
| 33 | CG56688-01 | 133 | 134 | Notch1-like |
| 34 | CG56715-01 | 135 | 136 | Olfactory Receptor-like |
| 35 | CG56718-01 | 137 | 138 | Olfactory Receptor-like |
| 36a | CG56729-01 | 139 | 140 | Cadherin 11-like |
| 36b | CG56729-02 | 141 | 142 | Cadherin 11-like |
| 37 | CG56733-01 | 143 | 144 | Ten-M2-like |
| 38 | CG56737-01 | 145 | 146 | Activin Beta C Chain-like |
| 39a | CG56737-02 | 147 | 148 | Activin Beta C Chain-like |
| 39b | CG56637-03 | 149 | 150 | Inhibin Beta E Chain-like |
| 40 | CG56097-01 | 151 | 152 | UDP Glycosyltransferase- |
| | | | | like |
| 41a | CG56680-01 | 153 | 154 | Sodium/Hydrogen Exchanger |
| | | | | 4-like |
| 41b | CG56680-02 | 155 | 156 | Sodium/Hydrogen Exchanger |
| | | | | 4-like |
| 42a | CG56682-01 | 157 | 158 | Kupffer Cell Receptor-like |
| 42b | CG56682-02 | 159 | 160 | Kupffer Cell Receptor-like |
| 42c | CG56682-03 | 161 | 162 | Kupffer Cell Receptor-like |
| 42d | CG56682-04 | 163 | 164 | Kupffer Cell Receptor-like |
| 43 | CG56690-01 | 165 | 166 | P2Y Purinoceptor-like |
| 44 | CG56692-01 | 167 | 168 | G Protein Coupled Receptor- |
| | | | | like |
| 45 | CG56694-01 | 169 | 170 | Mas Proto-Oncogene-like |
| 46a | CG56696-01 | 171 | 172 | Mas Proto-Oncogene-like |
| 46b | CG56696-02 | 173 | 174 | Mas-Related G Protein- |
| | | | | Coupled Receptor-like |
| 46c | CG56702-01 | 175 | 176 | Mas Proto-Oncogene-like |
| 46d | CG56698-01 | 177 | 178 | Mas Proto-Oncogene-like |
| 47 | CG56700-01 | 179 | 180 | Peptidyl-Prolyl Cis-Trans |
| | | | | Isomerase-like |
| 48a | CG56743-01 | 181 | 182 | Phospholipase C Delta-4- |
| | | | | like |
| 48b | CG56743-02 | 183 | 184 | Phospholipase C Delta-4- |
| | | | | like |
| 49 | CG56739-01 | 185 | 186 | Leukotriene-B4 Omega- |
| | | | | Hydroxylase-like |
| 50a | CG56771-01 | 187 | 188 | Protein Arginine N- |
| | | | | Methyltransferase 2-like |
| 50b | CG56771-02 | 189 | 190 | Protein Arginine N- |
| | | | | Methyltransferase 2-like |
| 51 | CG56759-01 | 191 | 192 | Olfactory Receptor-like |
| 52 | CG56731-01 | 193 | 194 | H326-like |
| 53 | CG56745-01 | 195 | 196 | Uracil |
| | | | | Phosphoribosyltransferase- |
| | | | | Like |
| 54a | CG56773-01 | 197 | 198 | Protein Phosphatase 2C-like |
| 54b | CG56773-02 | 199 | 200 | Protein Phosphatase 2C-like |
| 55 | CG56806-01 | 201 | 202 | Heparan Sulfate 6- |
| | | | | Sulfotransferase 3-like |
| 56a | CG56816-01 | 203 | 204 | N-Hydroxyarylamine |
| | | | | Sulfotransferase-like |
| 56b | CG56816-02 | 205 | 206 | N-Hydroxyarylamine |
| | | | | Sulfotransferase-like |
| 57 | CG56829-01 | 207 | 208 | Testis Specific Serine |
| | | | | Kinase-3-like |
| 58a | CG56315-01 | 209 | 210 | Gap Junction Beta-5-like |
| 58b | CG56315-02 | 211 | 212 | Connexin-like |
| 59 | CG56633-01 | 213 | 214 | Translation Initiation |
| | | | | Factor 5-like |
| 60a | CG56894-01 | 215 | 216 | Lynx1-like |
| 60b | CG56894-02 | 217 | 218 | Lynx1-like |
| 61 | CG56453-01 | 219 | 220 | Adlican-like |
| 62 | CG56781-01 | 221 | 222 | Neuropsin Precursor-like |
| 63 | CG53054-02 | 223 | 224 | Wnt-14 Precursor-like |
| 64 | CG56884-01 | 225 | 226 | Dipeptidyl peptidase-like |
| 65a | CG56651-01 | 227 | 228 | Protein phosphatase-like |
| 65b | CG56651-02 | 229 | 230 | Protein phosphatase-like |
| 66 | CG56313-01 | 231 | 232 | Olfactory receptor-like |
| 67 | CG56571-01 | 233 | 234 | Olfactory Receptor-Like |
| | | | | Protein OLF3-like |
| 68 | CG56844-01 | 235 | 236 | Endoglin (CD105 antigen)- |
| | | | | like |
| 69a | CG56950-01 | 237 | 238 | Interleukin 1 Epsilon-like |
| 69b | CG56136-02 | 239 | 240 | Interleukin 1 Epsilon-like |
| 70a | CG56878-01 | 241 | 242 | OS-9-like |
| 70b | CG56878-04 | 243 | 244 | OS-9-like |
| 71 | CG56906-01 | 245 | 246 | Sodium/Hydrogen Exchanger |
| | | | | 6-like |
| 72 | CG56910-01 | 247 | 248 | Ubiquitin-Specific |
| | | | | Protease-like |
| 73 | CG56822-01 | 249 | 250 | Sulfotransferase-like |
| 74 | CG56775-01 | 251 | 252 | Dual Specificity |
| | | | | Phosphatase-like |
| 75 | CG56783-01 | 253 | 254 | Dual Specificity |
| | | | | Phosphatase-like |
| 76a | CG56789-01 | 255 | 256 | Dual Specificity |
| | | | | Phosphatase-like |
| 76b | CG56789-02 | 257 | 258 | Dual Specificity |
| | | | | Phosphatase-like |
| 77 | CG56804-01 | 259 | 260 | Dual Specificity |
| | | | | Phosphatase-like |
| 78 | CG56810-01 | 261 | 262 | Dual Specificity |
| | | | | Phosphatase-like |
| 79 | CG56862-01 | 263 | 264 | Dual Specificity |
| | | | | Phosphatase-like |
| 80 | CG56882-01 | 265 | 266 | Dual Specificity |
| | | | | Phosphatase-like |
| 81a | CG56283-01 | 267 | 268 | Beta-1,3- |
| | | | | Galactosyltransferase-like |
| 81b | CG56283-02 | 269 | 270 | Beta-1,3- |
| | | | | Galactosyltransferase-like |
| 82 | CG56983-01 | 271 | 272 | Peptide YY-like |
| 83 | CG56890-01 | 273 | 274 | G Protein-Coupled Receptor |
| | | | | Kinase GRK7-like |
| 84 | CG56912-01 | 275 | 276 | Phospholipase C delta 1- |
| | | | | like |
| 85 | CG56955-01 | 277 | 278 | GTPase-Activating Protein- |
| | | | | like |
| 86 | CG56957-01 | 279 | 280 | GTPase-Activating Protein- |
| | | | | like |
| 87a | CG56886-01 | 281 | 282 | Rho-GTPase-Activating |
| | | | | Protein-like |
| 87b | CG56886-02 | 283 | 284 | Rho-GTPase-Activating |
| | | | | Protein-like |
| 88 | CG56394-01 | 285 | 286 | Glycerol-3-Phosphate |
| | | | | Dehydrogenase-like |
| 89 | CG56396-01 | 287 | 288 | Glycerol-3-Phosphate |
| | | | | Dehydrogenase-like |
| 90 | CG56888-01 | 289 | 290 | Serine/Threonine-Protein |
| | | | | Kinase PAK 2-like |
| 91 | CG56779-01 | 291 | 292 | D-Dopachrome Tautomerase- |
| | | | | like |
| 92 | CG56904-01 | 293 | 294 | Secreted leucine-rich |
| | | | | repeat (LRR)-like |
| 93 | CG56277-01 | 295 | 296 | Inosine-5′-Monophosphate |
| | | | | Dehydrogenase-like |
| 94 | CG56281-01 | 297 | 298 | Male-Specific Lethal 3-Like |
| | | | | 1-like |
| 95 | CG56975-01 | 299 | 300 | Cysteine Conjugate Beta- |
| | | | | Lyase-like |
| 96a | CG56918-01 | 301 | 302 | Monocarboxylate |
| | | | | transporter-like |
| 96b | CG56918-02 | 303 | 304 | Monocarboxylate |
| | | | | transporter-like |
| 96c | CG56918-03 | 305 | 306 | Sugar Transporter-like |
| 97a | CG57070-01 | 307 | 308 | Carboxypeptidase A1-like |
| 97b | CG57070-02 | 309 | 310 | Carboxypeptidase A1-like |
| 97c | CG57070-03 | 311 | 312 | Carboxypeptidase A1-like |
| 97d | CG57070-04 | 313 | 314 | Carboxypeptidase A1-like |
| 97e | CG57070-05 | 315 | 316 | Carboxypeptidase A1-like |
| 97f | CG57070-06 | 317 | 318 | Carboxypeptidase A1-like |
| 98 | CG56939-01 | 319 | 320 | Agrin-like |
| 99 | CG57010-01 | 321 | 322 | SNC73-like |

NOVX nucleic acids and their encoded polypeptides are useful in a variety of applications and contexts. The various NOVX nucleic acids and polypeptides according to the invention are useful as novel members of the protein families according to the presence of domains and sequence relatedness to previously described proteins. Additionally, NOVX nucleic acids and polypeptides can also be used to identify proteins that are members of the family to which the NOVX polypeptides belong.

NOV1, NOV3, and NOV4 are homologous to a Claudin-like family of proteins. Thus, the NOV1, NOV3, and NOV4 nucleic acids, polypeptides, antibodies and related compounds according to the invention will be useful in therapeutic and diagnostic applications implicated in various pathologies or conditions.

NOV2 is homologous to the Protein Serine Kinase-like family of proteins. Thus NOV2 nucleic acids, polypeptides, antibodies and related compounds according to the invention will be useful in therapeutic and diagnostic applications implicated in various pathologies or conditions.

NOV5 is homologous to a family of Monocarboxylate transporter-like proteins. Thus, the NOV5 nucleic acids and polypeptides, antibodies and related compounds according to the invention will be useful in therapeutic and diagnostic applications implicated in various pathologies or conditions

NOV6 is homologous to the nitrilase-1-like family of proteins. Thus, NOV6 nucleic acids, polypeptides, antibodies and related compounds according to the invention will be useful in therapeutic and diagnostic applications implicated in various pathologies or conditions.

NOV7 is homologous to the Cleavage Signal-1-like family of proteins. Thus NOV7 nucleic acids, polypeptides, antibodies and related compounds according to the invention will be useful in therapeutic and diagnostic applications implicated in various pathologies or conditions.

NOV8 is homologous to the Matripase-like family of proteins. Thus NOV8 nucleic acids, polypeptides, antibodies and related compounds according to the invention will be useful in therapeutic and diagnostic applications implicated in, various pathologies or conditions.

NOV9 is homologous to members of the Neuropeptide Y/Peptide YY receptor-like family of proteins. Thus, the NOV9 nucleic acids, polypeptides, antibodies and related compounds according to the invention will be useful in therapeutic and diagnostic applications implicated in various pathologies or conditions.

NOVs10 through 20,, NOV43, NOV44, and NOV83 are homologous to the G-Protein Coupled Receptor-like family of proteins. Thus, these nucleic acids and polypeptides, antibodies and related compounds according to the invention will be useful in therapeutic and diagnostic applications implicated in various pathologies or conditions.

NOV21 and NOV22 are homologous to the Adrenal; secretory serine protease like growth factor binding protein-like family of proteins. Thus, NOV21 and NOV22 nucleic acids and polypeptides, antibodies and related compounds according to the invention will be useful in therapeutic and diagnostic applications implicated in various pathologies or conditions.

NOV23 is homologous to the Serine Protease DESC-1-like family of proteins. Thus, NOV23 nucleic acids and polypeptides, antibodies and related compounds according to the invention will be useful in various pathologies or conditions.

NOV24 is homologous to the Parchorin-like family of proteins. Thus, NOV24 nucleic acids and polypeptides, antibodies and related compounds according to the invention will be useful in therapeutic and diagnostic applications implicated in various pathologies or disorders.

NOV25 is homologous to theProtein Phosphatase-like family of proteins. Thus, NOV25 nucleic acids and polypeptides, antibodies and related compounds according to the invention will be useful in therapeutic and diagnostic applications implicated in various pathologies or conditions.

NOV26 is homologous to the GAGE7-like family of proteins. Thus, NOV26 nucleic acids and polypeptides, antibodies and related compounds according to the invention will be useful in therapeutic and diagnostic applications implicated in various pathologies/disorders.

NOV27 is homologous to the Sodium-Glucose Cotransporter-like family of proteins. Thus, NOV27 nucleic acids and polypeptides, antibodies and related compounds according to the invention will be useful in therapeutic and diagnostic applications implicated in various pathologies or conditions

NOV28 is homologous to the MYD-1-like family of proteins. Thus, NOV28 nucleic acids and polypeptides, antibodies and related compounds according to the invention will be useful in therapeutic and diagnostic applications implicated in various pathologies or conditions

NOV29 is homologous to the CRAL-TRIO-like family of proteins. Thus, NOV27 nucleic acids and polypeptides, antibodies and related compounds according to the invention will be useful in therapeutic and diagnostic applications implicated in various pathologies or conditions

NOV30 is homologous to the Ryudocan-like family of proteins. Thus, NOV30 nucleic acids and polypeptides, antibodies and related compounds according to the invention will be useful in therapeutic and diagnostic applications implicated in various pathologies or conditions

NOV31 is homologous to the Sulfur-rich Keratin-like family of proteins. Thus, NOV31 nucleic acids and polypeptides, antibodies and related compounds according to the invention will be useful in therapeutic and diagnostic applications implicated in various pathologies or conditions

NOV32 is homologous to the DMNT1 associated protein-like family of proteins. Thus, NOV32 nucleic acids and polypeptides, antibodies and related compounds according to the invention will be useful in therapeutic and diagnostic applications implicated in various pathologies or conditions

NOV33 is homologous to the Notch1-like family of proteins. Thus, NOV33 nucleic acids and polypeptides, antibodies and related compounds according to the invention will be useful in therapeutic and diagnostic applications implicated in various pathologies or conditions

NOV34, NOV35, NOV51, NOV66, and NOV67 are homologous to the Olfactory Receptor-like family of proteins. Thus, NOV34, NOV35, NOV51, NOV66, and NOV67 nucleic acids and polypeptides, antibodies and related compounds according to the invention will be useful in therapeutic and diagnostic applications implicated in various pathologies or conditions

NOV36 is homologous to the Cadherin 11-like family of proteins. Thus, NOV36 nucleic acids and polypeptides, antibodies and related compounds according to the invention will be useful in therapeutic and diagnostic applications implicated in various pathologies or conditions

NOV37 is homologous to the Ten-M2-like family of proteins. Thus, NOV33 nucleic acids and polypeptides, antibodies and related compounds according to the invention will be useful in therapeutic and diagnostic applications implicated in various pathologies or conditions

NOV38 and NOV39 are homologous to the Activin/Inhibin-like family of proteins. Thus, NOV38 and NOV39 nucleic acids and polypeptides, antibodies and related compounds according to the invention will be useful in therapeutic and diagnostic applications implicated in various pathologies or conditions

NOV40 is homologous to the UDP Glycosyltransferase-like family of proteins. Thus, NOV40 nucleic acids and polypeptides, antibodies and related compounds according to the invention will be useful in therapeutic and diagnostic applications implicated in various pathologies or conditions

NOV41 is homologous to the Sodium/Hydrogen Exchanger 4-like family of proteins. Thus, NOV41 nucleic acids and polypeptides, antibodies and related compounds according to the invention will be useful in therapeutic and diagnostic applications implicated in various pathologies or conditions

NOV42 is homologous to the Kupffer Cell Receptor-like family of proteins. Thus, NOV42 nucleic acids and polypeptides, antibodies and related compounds according to the invention will be useful in therapeutic and diagnostic applications implicated in various pathologies or conditions

NOV45 and NOV46 is homologous to the Mas Proto-Oncogene-like family of proteins. Thus, NOV45 and NOV46 nucleic acids and polypeptides, antibodies and related compounds according to the invention will be useful in therapeutic and diagnostic applications implicated in various pathologies or conditions

NOV47 is homologous to the Peptidyl-Prolyl Cis-Trans Isomerase-like family of proteins. Thus, NOV47 nucleic acids and polypeptides, antibodies and related compounds according to the invention will be useful in therapeutic and diagnostic applications implicated in various pathologies or conditions

NOV48 is homologous to the Phospholipase C Delta-4-like family of proteins. Thus, NOV48 nucleic acids and polypeptides, antibodies and related compounds according to the invention will be useful in therapeutic and diagnostic applications implicated in various pathologies or conditions

NOV49 is homologous to the Leukotriene-B4 Omega Hydroxylase-like family of proteins. Thus, NOV49 nucleic acids and polypeptides, antibodies and related compounds according to the invention will be useful in therapeutic and diagnostic applications implicated in various pathologies or conditions

NOV50 is homologous to the Protein Arginine N-Methyltransferase 2-like family of proteins. Thus, NOV50 nucleic acids and polypeptides, antibodies and related compounds according to the invention will be useful in therapeutic and diagnostic applications implicated in various pathologies or conditions

NOV52 is homologous to the H326-like family of proteins. Thus, NOV52 nucleic acids and polypeptides, antibodies and related compounds according to the invention will be useful in therapeutic and diagnostic applications implicated in various pathologies or conditions

NOV53 is homologous to the Uracil Phosphoribosyltransferase-like family of proteins. Thus, NOV53 nucleic acids and polypeptides, antibodies and related compounds according to the invention will be useful in therapeutic and diagnostic applications implicated in various pathologies or conditions

NOV54 is homologous to the Protein Phosphatase 2C-like family of proteins. Thus, NOV54 nucleic acids and polypeptides, antibodies and related compounds according to the invention will be useful in therapeutic and diagnostic applications implicated in various pathologies or conditions

NOV55 is homologous to the Heparan Sulfate 6-Sulfotransferase 3-like family of proteins. Thus, NOV55 nucleic acids and polypeptides, antibodies and related compounds according to the invention will be useful in therapeutic and diagnostic applications implicated in various pathologies or conditions

NOV56 is homologous to the N-Hydroxyarylamine Sulfotransferase 3-like family of proteins. Thus, NOV52 nucleic acids and polypeptides, antibodies and related compounds according to the invention will be useful in therapeutic and diagnostic applications implicated in various pathologies or conditions

NOV57 is homologous to the Testis Specific Serine Kinase-3-like family of proteins. Thus, NOV57 nucleic acids and polypeptides, antibodies and related compounds according to the invention will be useful in therapeutic and diagnostic applications implicated in various pathologies or conditions

NOV58 is homologous to the Gap Junction Beta-5-like family of proteins. Thus, NOV58 nucleic acids and polypeptides, antibodies and related compounds according to the invention will be useful in therapeutic and diagnostic applications implicated in various pathologies or conditions

NOV59 is homologous to the Translation Initiation Factor 5-like family of proteins. Thus, NOV59 nucleic acids and polypeptides, antibodies and related compounds according to the invention will be useful in therapeutic and diagnostic applications implicated in various pathologies or conditions

NOV60 is homologous to the Lynx1-like family of proteins. Thus, NOV60 nucleic acids and polypeptides, antibodies and related compounds according to the invention will be useful in therapeutic and diagnostic applications implicated in various pathologies or conditions

NOV61 is homologous to the Adlican-like family of proteins. Thus, NOV61 nucleic acids and polypeptides, antibodies and related compounds according to the invention will be useful in therapeutic and diagnostic applications implicated in various pathologies or conditions

NOV62 is homologous to the Neuropsin Precursor-like family of proteins. Thus, NOV62 nucleic acids and polypeptides, antibodies and related compounds according to the invention will be useful in therapeutic and diagnostic applications implicated in various pathologies or conditions

NOV63 is homologous to the Wnt-14-like family of proteins. Thus, NOV63 nucleic acids and polypeptides, antibodies and related compounds according to the invention will be useful in therapeutic and diagnostic applications implicated in various pathologies or conditions

NOV64 is homologous to the Dipeptidyl peptidase-like family of proteins. Thus, NOV64 nucleic acids and polypeptides, antibodies and related compounds according to the invention will be useful in therapeutic and diagnostic applications implicated in various pathologies or conditions

NOV65 is homologous to the Protein phosphatase-like family of proteins. Thus, NOV65 nucleic acids and polypeptides, antibodies and related compounds according to the invention will be useful in therapeutic and diagnostic applications implicated in various pathologies or conditions

NOV68 is homologous to the Endoglin (CD105 antigen)-like family of proteins. Thus, NOV68 nucleic acids and polypeptides, antibodies and related compounds according to the invention will be useful in therapeutic and diagnostic applications implicated in various pathologies or conditions

NOV69 is homologous to the Interleukin 1 Epsilom-like family of proteins. Thus, NOV69 nucleic acids and polypeptides, antibodies and related compounds according to the invention will be useful in therapeutic and diagnostic applications implicated in various pathologies or conditions

NOV70 is homologous to the OS-9-like family of proteins. Thus, NOV70 nucleic acids and polypeptides, antibodies and related compounds according to the invention will be useful in therapeutic and diagnostic applications implicated in various pathologies or conditions

NOV71 is homologous to the Sodium/Hydrogen Exchanger 6-like family of proteins. Thus, NOV71 nucleic acids and polypeptides, antibodies and related compounds according to the invention will be useful in therapeutic and diagnostic applications implicated in various pathologies or conditions

NOV72 is homologous to the Ubiquitin Specific Protease-like family of proteins. Thus, NOV72 nucleic acids and polypeptides, antibodies and related compounds according to the invention will be useful in therapeutic and diagnostic applications implicated in various pathologies or conditions

NOV73 is homologous to the Sulfotransferase-like family of proteins. Thus, NOV73 nucleic acids and polypeptides, antibodies and related compounds according to the invention will be useful in therapeutic and diagnostic applications implicated in various pathologies or conditions

NOV74, NOV75, NOV76, NOV77, NOV78, NOV79, and NOV80 are homologous to the Dual Specificity Phosphatase-like family of proteins. Thus, NOV74, NOV75, NOV76, NOV77, NOV78, NOV79, and NOV80 nucleic acids and polypeptides, antibodies and related compounds according to the invention will be useful in therapeutic and diagnostic applications implicated in various pathologies or conditions

NOV81 is homologous to the Beta-1,3-Galactosyltransferase-like family of proteins. Thus, NOV81 nucleic acids and polypeptides, antibodies and related compounds according to the invention will be useful in therapeutic and diagnostic applications implicated in various pathologies or conditions

NOV82 is homologous to the Peptide YY-like family of proteins. Thus, NOV82 nucleic acids and polypeptides, antibodies and related compounds according to the invention will be useful in therapeutic and diagnostic applications implicated in various pathologies or conditions

NOV84 is homologous to the Phospholipase C delta 1-like family of proteins. Thus, NOV84 nucleic acids and polypeptides, antibodies and related compounds according to the invention will be useful in therapeutic and diagnostic applications implicated in various pathologies or conditions

NOV85, NOIV86, and NOV87 are homologous to the GTPase-Activating Protein-like family of proteins. Thus, NOV85, NOIV86, and NOV87 nucleic acids and polypeptides, antibodies and related compounds according to the invention will be useful in therapeutic and diagnostic applications implicated in various pathologies or conditions

NOV88 and NOV89 are homologous to the Glyceroil-3-Phosphate Dehydrogenase-like family of proteins. Thus, NOV88 and NOV89 nucleic acids and polypeptides, antibodies and related compounds according to the invention will be useful in therapeutic and diagnostic applications implicated in various pathologies or conditions

NOV90 is homologous to the Serine/Threonine-Protein Kinase PAK 2-like family of proteins. Thus, NOV90 nucleic acids and polypeptides, antibodies and related compounds according to the invention will be useful in therapeutic and diagnostic applications implicated in various pathologies or conditions

NOV91 is homologous to the D-Dopachrome Tautomerase family of proteins. Thus, NOV91 nucleic acids and polypeptides, antibodies and related compounds according to the invention will be useful in therapeutic and diagnostic applications implicated in various pathologies or conditions

NOV92 is homologous to the Secreted leucine-rich repeat (LRR)-like family of proteins. Thus, NOV92 nucleic acids and polypeptides, antibodies and related compounds according to the invention will be useful in therapeutic and diagnostic applications implicated in various pathologies or conditions

NOV93 is homologous to the Inosine-5′-Monophosphate Dehydrogenase-like family of proteins. Thus, NOV93 nucleic acids and polypeptides, antibodies and related compounds according to the invention will be useful in therapeutic and diagnostic applications implicated in various pathologies or conditions

NOV94 is homologous to the Male-Specific Lethal 3-like family of proteins. Thus, NOV94 nucleic acids and polypeptides, antibodies and related compounds according to the invention will be useful in therapeutic and diagnostic applications implicated in various pathologies or conditions

NOV95 is homologous to the Cysteine Conjugate Beta Lyase-like family of proteins. Thus, NOV95 nucleic acids and polypeptides, antibodies and related compounds according to the invention will be useful in therapeutic and diagnostic applications implicated in various pathologies or conditions

NOV96 is homologous to the Monocarboxylate transporter-like family of proteins. Thus, NOV96 nucleic acids and polypeptides, antibodies and related compounds according to the invention will be useful in therapeutic and diagnostic applications implicated in various pathologies or conditions

NOV97 is homologous to the Carboxypeptidase A1-like family of proteins. Thus, NOV97 nucleic acids and polypeptides, antibodies and related compounds according to the invention will be useful in therapeutic and diagnostic applications implicated in various pathologies or conditions

NOV98 is homologous to the Agrin-like family of proteins. Thus, NOV98 nucleic acids and polypeptides, antibodies and related compounds according to the invention will be useful in therapeutic and diagnostic applications implicated in various pathologies or conditions

NOV99 is homologous to the SNC73-like family of proteins. Thus, NOV99 nucleic acids and polypeptides, antibodies and related compounds according to the invention will be useful in therapeutic and diagnostic applications implicated in various pathologies or conditions

The NOVX nucleic acids and polypeptides can also be used to screen for molecules, which inhibit or enhance NOVX activity or function. Specifically, the nucleic acids and polypeptides according to the invention may be used as targets for the identification of small molecules that modulate or inhibit, e.g., neurogenesis, cell differentiation, cell proliferation, hematopoiesis, wound healing and angiogenesis.

Additional utilities for the NOVX nucleic acids and polypeptides according to the invention are disclosed herein.

NOV1

NOV1 includes three novel human 1 Claudin-like proteins disclosed below. The disclosed sequences have been named NOV1a, NOV1b, NOV1c, NOV1d, NOV1e, NOV1f, and NOV1g.

NOV1a

A disclosed NOV1a nucleic acid of 687 nucleotides (also referred to as CG56592-02) encoding a novel human Claudin 6-like protein is shown in Table 1A An open reading frame was identified beginning with an ATG initiation codon at nucleotides 6-8 and ending with a TAG termination codon at nucleotides 678-680. The start and stop codons are in bold letters in Table 1A, and the 5′ and 3′ untranslated regions are underlined.

TABLE 1A
NOV1a nucleotide sequence.

(SEQ ID NO:1)

TGACTATGGCCTGGAGTTTCCGTGCAAAAGTCCAGCTCGGGGGGCTACTTCTCTCCCTCCTTGGCTGGGTCT
GCTCCTGTGTTACCACCATCCTGCCCCAGTGGAAGACTCTTAATCTGGAACTGAACGAGATGGAGACCTGGA
TCATGGGGATTTGGGAGGTCTGCGTGGATCGAGAGGAAGTCGCCACTGTGTGCAAGGCCTTTGAATCCTTCT
TGTCTCTGCCCCAGGAGCTCCAGGTAGCCCGCATCCTCATGGTAGCCTCCCATGGGCTGGGCCTATTGGGGC
TTTTGCTCTGCAGCTTTGGGTCTGAATGCTTCCAGTTTCACAGGATCAGATGGGTATTCAAGAGGCGGCTTG
GTCTCCTGGGAAGGACTTTGGAGGCATCCGCTTCAGCCACTACCCTCCTTCCAGTCTCCTGGGTGGCCCATG
CCACAATCCAAGACTTCTGGGATGACAGCATCCCTGACATCATACCCTCGGTGGGAGTTTGGAGGTGCCCTC
TACTTGGGCTGGGCTGCTGGTATTTTCCTGGCTCTTGGTGGGCTACTCCTCATCTTCTCGGCCTGCCTGGGA
AAAGAAGATGTGCCTTTTCCTTTGATGGCTGGTCCCACAGTCCCCCTATCCTGTGCTCCAGTGGAGGAGTCA
GATGGCTCCTTCCACCTCATGCTAAGACCTAGGAACCTG

In a search of public sequence databases, the NOV1a nucleic acid sequence, located on chromsome 12 has 337 of 534 bases (63%) identical to a gb:GENBANK-ID:HSA249735|acc:AJ249735.1 mRNA from Homo sapiens (CLDN6 gene for claudin-6).

In all BLAST alignments herein, the “E-value” or “Expect” value is a numeric indication of the probability that the aligned sequences could have achieved their similarity to the BLAST query sequence by chance alone, within the database that was searched. For example, the probability that the subject (“Sbjct”) retrieved from the NOV1a BLAST analysis, e.g., Homo sapiens CLDN6 gene for claudin-6, matched the Query NOV1a sequence purely by chance is 1.4e⁻¹⁵. The Expect value (E) is a parameter that describes the number of hits one can “expect” to see just by chance when searching a database of a particular size. It decreases exponentially with the Score (S) that is assigned to a match between two sequences. Essentially, the E value describes the random background noise that exists for matches between sequences.

The Expect value is used as a convenient way to create a significance threshold for reporting results. The default value used for blasting is typically set to 0.0001. In BLAST 2.0, the Expect value is also used instead of the P value (probability) to report the significance of matches. For example, an E value of one assigned to a hit can be interpreted as meaning that in a database of the current size one might expect to see one match with a similar score simply by chance. An E value of zero means that one would not expect to see any matches with a similar score simply by chance. See, e.g., http://www.ncbi.nlm.nih.gov/Education/BLASTinfo/. Occasionally, a string of X's or N's will result from a BLAST search. This is a result of automatic filtering of the query for low-complexity sequence that is performed to prevent artifactual hits. The filter substitutes any low-complexity sequence that it finds with the letter “N” in nucleotide sequence (e.g., “NNNNNNNNNNNNN”) or the letter “X” in protein sequences (e.g., “XXXXXXXXX”). Low-complexity regions can result in high scores that reflect compositional bias rather than significant position-by-position alignment. (Wootton and Federhen, Methods Enzymol 266:554-571, 1996).

The disclosed NOV1a polypeptide (SEQ ID NO:2) encoded by SEQ ID NO:1 has 229 amino acid residues and is presented in Table 1B using the one-letter amino acid code. Signal P, Psort and/or Hydropathy results predict that NOV1a has no signal peptide and is likely to be localized the plasma membrane with a certainty of 0.6400. Alternatively, NOV1a also may localize to the Golgi body with acertainty of 0.4600, the endoplasmic reticulum (membrane) with a certainty of 0.3700 or in the endoplasmic reticulum (lumen) with a certainty of 0.1000. The most likely cleavage site for a NOV1a peptide is between amino acids 24 and 25, at: VCS-CV.

TABLE 1B
Encoded NOV1a protein sequence.

(SEQ ID NO:2)

MAWSFRAKVQLGGLLLSLLGWVCSCVTTILPQWKTLNLELNEMETWIMGIWEVCVDREEVATVCKAFESFLS
LPQELQVARILMVASHGLGLLCLLLCSFGSECFQFHRIRWVFKRRLGLLGRTLEASASATTLLPVSWVAHAT
IQDFWDDSIPDIIPRWEFGGALYLGWAAGIFLALGGLLLIFSACLGKEDVPFPLMAGPTVPLSCAPVEESDG
SFHLMLRPRNLVI

A search of sequence databases reveals that the NOV1a amino acid sequence has 81 of 207 amino acid residues (39%) identical to, and 111 of 207 amino acid residues (53%) similar to, the 219 amino acid residue ptnr:SWISSPROT-ACC:Q9Z262 protein from Mus musculus (Mouse) (Claudin-6) (E=2.7e⁻²⁷).

NOV1a is predicted to be expressed in Bone Marrow, Brain, Liver, Placenta, and Lung.

NOV1b

A disclosed NOV1b nucleic acid of 687 nucleotides (also referred to as CG56586-01) encoding a human Claudin-3-like protein is shown in Table 1C. An open reading frame was identified beginning with an ATG initiation codon at nucleotides 6-8 and ending with a TAG codon at nucleotides 678-680. Putative untranslated regions upstream from the initiation codon, and downstream from the termination codon, if any, are underlined in Table 1C. The start and stop codons are in bold letters.

TABLE 1C
NOV1b nucleotide sequence.

(SEQ ID NO:3)

TGACTATGGCCTGGAGTTTCCGTGCAAAAGTCCAGCTCGGGGGGCTACTTCTCTCCCTCCTTGGCTGGGTCT
GCTCCTGTGTTACCACCATCCTGCCCCAGTGGAAGACTCTTAATCTGGAACTGAACGAGATGGAGACCTGGA
TCATGGGGATTTGGGAGGTCTGCGTGGATCGAGAGGAAGTCGCCACTGTGTGCAAGGCCTTTGAATCCTTCT
TGTCTCTGCCCCAGGAGCTCCAGGTAGCCCGCATCCTCATGGTAGCCTCCCATGGGCTGGGCCTATTGGGGC
TTTTGCTCTGCAGCTTTGGGTCTGAATGCTTCCAGTTTCACAGGATCAGATGGGTATTCAAGAGGCGGCTTG
GTCTCCTGGGAAGGACTTTGGAGGCATCCGCTTCAGCCACTACCCTCCTTCCAGTCTCCTGGGTGGCCCATG
CCACAATCCAAGACTTCTGGGATGACAGCATCCCTGACATCATACCCTCGGTCGGAGTTTGGAGGTGCCCTC
TACTTGGGCTGGGCTGCTGGTATTTTCCTGGCTCTTGGTGGGCTACTCCTCATCTTCTCGGCCTGCCTGGGA
AAAGAAGATGTGCCTTTTCCTTTGATGGCTGGTCCCACAGTCCCCCTATCCTGTGCTCCAGTGGAGGAGTCA
GATGGCTCCTTCCACCTCATGCTAAGACCTAGGAACCTG

In a search of public sequence databases, the NOV1b nucleic acid sequence, located on chromsome 11 is 338 of 534 bases (63%) identical to a gb:GENBANK-ID:HSA249735|acc:AJ249735.1 mRNA from Homo sapiens (CLDN6 gene for claudin-6). (E=2.8e⁻¹⁶).

The disclosed NOV1b polypeptide (SEQ ID NO:4) encoded by SEQ ID NO:3 has 224 amino acid residues and is presented in Table 1D using the one-letter amino acid code. Signal P, Psort and/or Hydropathy results predict that NOV1b has a signal peptide and is likely to be localized in the plasma membrane with a certainty of 0.4600. Alternatively, NOV1b may also localize to the microbody (peroxisome) with acertainty of 0.3200, the endoplasmic reticulum (membrane) with a certainty of 0.1000 or in the endoplasmic reticulum (lumen) with a certainty of 0.1000. The most likely cleavage site for a NOV1b peptide is between amino acids 24 and 25, at: VCS-CV.

TABLE 1D
Encoded NOV1b protein sequence.

(SEQ ID NO:4)

MAWSFRAKVQLGGLLLSLLGWVCSCVTTILPQWKTLNLELNEMETWIMGIWEVCVDREEVATVCKAFESFLS
LPQELQVARILMVASHGLGLLGLLLCSFGSECFQFHRIRWVFKRRLGLLGRTLEASASATTLLPVSWVAHAT
IQDFWDDSIPDIIPSVGVWRCPLLGLGCWYFPCSWWATPHLLGLPGKRRCAFSFDGWSHSPPILCSSGGVRW
LLPPHAKT

A search of sequence databases reveals that the NOV1b amino acid sequence has 50 of 149 amino acid residues (33%) identical to, and 83 of 149 amino acid residues (55%) similar to, the 219 amino acid residue ptnr:SWISSPROT-ACC:Q63400 protein from Rattus norvegicus (Rat) (Claudin-3 (Ventral Prostate.1 Protein) (RVP1)) (E=0.0).

NOV1b is predicted to be expressed in Bone Marrow, Brain, Liver, Placenta, and Lung.

NOV1c

A disclosed NOV1c nucleic acid of 642 nucleotides (also referred to as CG56592-03) encoding a novel Claudin-6-like protein is shown in Table 1E. An open reading frame was identified beginning with a ATG initiation codon at nucleotides 6-8 and ending with a TAG codon at nucleotides 609-611. The start and stop codons are in bold letters, and the 5′ and 3′ untranslated regions are underlined.

TABLE 1E
NOV1c Nucleotide Sequence

(SEQ ID NO:5)

TGACTATGGCCTGGAGTTTCCGTGCAAAAGTCCAGCTCGGGGGGCTACTTCTCTCCCTCCTTGGCTGGGTC
TGCTCCTGTGTTACCACCATCCTGCCCCAGTGGAAGACTCTTAATCTGGAACTGAACGAGATGGAGACCTG
GATCATGGGGATTTGGGAGGTCTGCGTGGATCGAGAGGAAGTCGCCACTGTGTGCAAGGCCTTTGAATCCT
TCTTGTCTCTGCCCCAGGAGCTCCAGTTTCACAGGATCAGATGGGTATTCAAGAGGCGGCTTGGTCTCCTG
GGAAGGACTTTGGAGGCATCCGCTTCAGCCACTACCCTCCTTCCAGTCTCCTGGGTGGCCCATCCCACAAT
CCAAGACTTCTGGGATGACAGCATCCCTGACATCATACCTCGGTGGGAGTTTGGAGGTGCCCTCTACTTGG
GCTGGGCTGCTGGTATTTTCCTGGCTCTTGGTGGGCTACTCCTCATCTTCTCGGCCTGCCTGGGAAAAGAA
GATGTGCCTTTTCCTTTGATGGCTGGTCCCACAGTCCCCCTATCCTGTGCTCCAGTGGAGGAGTCAGATGG
CTCCTTCCACCTCATGCTAAGACCTAGGAACCTGGTCATCTAGGACTGGCTTCTGCCAAGGATCTCTGGAA
TAA

The disclosed NOV1c nucleic acid sequence maps to chromosome 12 and has 144 of 220 bases (65%) identical to a gb:GENBANK-ID:HSA249735|acc:AJ249735.1 mRNA from Homo sapiens (CLDN6 gene for claudin-6) (E=0.0).

A disclosed NOV1c protein (SEQ ID NO:6) encoded by SEQ ID NO:5 has 201 amino acid residues, and is presented using the one-letter code in Table 1F. Signal P, Psort and/or Hydropathy results predict that NOV1c does have a signal peptide, and is likely to be localized to the plasma membrane with a certainty of 0.4600. In other embodiments NOV1c is also likely to be localized to the microbody (peroxisome) with a certainty of 0.2651, to endoplasmic reticulum (membrane) with a certainty of 0.1000, or to the endoplasmic reticulum (lumen) with a certainty of 0.1000. The most likely cleavage site for NOV1c is between positions 24 and 25, (VCS-CV).

TABLE 1F
Encoded NOV1c protein sequence.

(SEQ ID NO:6)

MAWSFRAKVQLGGLLLSLLGWVCSCVTTILPQWKTLNLELNEMETWIMGIWEVCVDREEVATVCKAFESFL
SLPQELQFHRIRWVFKRRLGLLGRTLEASASATTLLPVSWVAHATIQDFWDDSIPDIIPRWEFGGALYLGW
AAGIFLALGGLLLIFSACLGKEDVPFPLMAGPTVPLSCAPVEESDGSFHLMLRPRNLVI

The disclosed NOV1c amino acid has 55 of 94 amino acid residues (58%) identical to, and 62 of 94 amino acid residues (65%) similar to, the 220 amino acid residue ptnr:SPTREMBL-ACC:Q9D7U6 protein from Mus musculus (Mouse) (2210404A22RIK Protein) (E=3.1e⁻⁴⁷).

In addition, NOV1c is predicted to be expressed in Bone Marrow, Brain, Liver, Placenta, and Lung.

NOV1d

A disclosed NOV1d nucleic acid of 726 nucleotides (also referred to as CG56592-02) encoding a novel Claudin 6-like protein is shown in Table 1G. An open reading frame was identified beginning with an ATG codon at nucleotides 6-8 and ending with a TAG codon at nucleotides 693-695. The start and stop codons are in bold letters and the 5′ and 3′ untranslated regions are underlined in Table 1G.

TABLE 1G
NOV1d nucleotide sequence

(SEQ ID NO:7)

TGACTATGGCCTGGAGTTTCCGTGCAAAAGTCCAGCTCGGGGGGCTACTTCTCTCCCTCCTTGGCTGGGTCT
GTTCCTGTGTTACCACCATCCTGCCCCAGTGGAAGACTCTTAATCTGGAACTGAACGAGATGGAGACCTGGA
TCATGGGGATTTGGGAGGTCTGCGTGGATCGAGAGGAAGTCGCCACTGTGTGCAAGGCCTTTGAATCCTTCT
TGTCTCTGCCCCAGGAGCTCCAGGTAGCCCGCATCCTCATGGTAGCCTCCCATGGGCTGGGCCTATTGGGGC
TTTTGCTCTGCAGCTTTGGGTCTGAATGCTTCCAGTTTCACAGGATCAGATGGGTATTCAAGAGGCGGCTTG
GTCTCCTGGGAAGGACTTTGGAGGCATCCGCTTCAGCCACTACCCTCTTTCCAGTCTCCTGGGTGGCCCATG
CCACAATCCAAGACTTCTGGGATGACAGCATCCCTGACATCATACCTCGGTGGGAGTTTGGAGGTGCCCTCT
ACTTGGGCTGGGCTGCTGGTATTTTCCTGGCTCTTGGTGGGCTACTCCTCATCTTCTCGGCCTGCCTGGGAA
AAGAAGATGTGCCTTTTCCTTTGATGGCTGGTCCCACAGTCCCCCTATCCTGTGCTCCAGTGGAGGAGTCAG
ATGGCTCCTTCCACCTCATGCTAAGACCTAGGAACCTGGTCATCTAGGACTGGCTTCTGCCAAGGATCTCTG
GAATAA

In a search of public sequence databases, the NOV1d nucleic acid sequence, located on chromsome 12 has 336 of 534 bases (62%) identical to a gb:GENBANK-ID:HSA249735|acc:AJ249735.1 mRNA from Homo sapiens (CLDN6 gene for claudin-6) (E=6.5e⁻¹⁶).

The disclosed NOV1d polypeptide (SEQ ID NO:8) encoded by SEQ ID NO:7 has 229 amino acid residues and is presented in Table 1H using the one-letter amino acid code. Signal P, Psort and/or Hydropathy results predict that NOV1d has no signal peptide and is likely to be localized the the plasma membrane with a certainty of 0.6400. Alternatively, NOV1d may also localize to the Golgi body with acertainty of 0.4600, the endoplasmic reticulum (membrane) with a certainty of 0.3700 or in the endoplasmic reticulum (lumen) with a certainty of 0.1000. The most likely cleavage site for a NOV1d peptide is between amino acids 24 and 25, at: VCS-CV.

TABLE 1H
Encoded NOV1d protein sequence.

(SEQ ID NO:8)

MAWSFRAKVQLGGLLLSLLGWVCSCVTTILPQWKTLNLELNEMETWIMGIWEVCVDREEVATVCKAFESFLS
LPQELQVARILMVASHGLGLLGLLLCSFGSECFQFHRIRWVFKRRLGLLGRTLEASASATTLLPVSWVAHAT
IQDFWDDSIPDIIPRWEFGGALYLGWAAGIFLALGGLLLIFSACLGKEDVPFPLMAGPTVPLSCAPVEESDG
SFHLMLRPRNLVI

A search of sequence databases reveals that the NOV1d amino acid sequence has 81 of 207 amino acid residues (39%) identical to, and 111 of 207 amino acid residues (53%) similar to, the 219 amino acid residue ptnr:SWISSPROT-ACC:Q9Z262 protein from Mus musculus (Mouse) (Claudin-6) (E=2.8e⁻²⁷).

Expression information was derived from the tissue sources of the sequences that were included in the derivation of NOV1d. The sequence is predicted to be expressed in Bone Marrow, Brain, Liver, Placenta, and Lung.

Homologies to any of the above NOV1 proteins will be shared by the other NOV1 proteins insofar as they are homologous to each other as shown below. Any reference to NOV1 is assumed to refer to all four of the NOV1 proteins in general, unless otherwise noted.

The disclosed NOV1a polypeptide has homology to the amino acid sequences shown in the BLASTP data listed in Table 1I.

TABLE 1I
BLAST results for NOV1a

| Gene Index/ | | Length | Identity | Positives | |
|---|---|---|---|---|---|
| Identifier | Protein/Organism | (aa) | (%) | (%) | Expect |
| gi\|17458947\|ref\|XP_{—} | similar to | 229 | 229/229 | 229/229 | e−109 |
| 061964.1\| | putative (H. | | (100%) | (100%) | |
| (XM_061964) | sapiens) [Homo | | | | |
| | sapiens] | | | | |
| >gi\|17437506\|ref\|XP_{—} | similar to | 220 | 99/172 | 125/172 | 4e−50 |
| 068031.1\| | putative (H. | | (57%) | (72%) | |
| (XM_068031) | sapiens) [Homo | | | | |
| | sapiens] | | | | |
| gi\|17437504\|ref\|XP_{—} | similar to | 220 | 99/172 | 126/172 | 4e−43 |
| 068030.1\| | putative (H. | | (57%) | (72%) | |
| (XM_068030) | sapiens) [Homo | | | | |
| | sapiens] | | | | |
| gi\|12843248\|dbj\|BAB25914.1\| | PMP- | 220 | 104/188 | 131/188 | 3e−40 |
| (AK008821) | 22/EMP/MP20/Claud | | (55%) | (69%) | |
| | in family | | | | |
| | containing | | | | |
| | protein˜data | | | | |
| | source: Pfam, | | | | |
| | source | | | | |
| | key: PF00822, | | | | |
| | evidence: ISS˜putative | | | | |
| | [Mus | | | | |
| | musculus] | | | | |
| gi\|7710002\|ref\|NP_057883.1\| | claudin 1 [Mus | 211 | 67/194 | 99/194 | 2e−20 |
| (NM_016674) | musculus] | | (34%) | (50%) | |

The homology between these and other sequences is shown graphically in the ClustalW analysis shown in Table 1J. In the ClustalW alignment of the NOV1 proteins, as well as all other ClustalW analyses herein, the black outlined amino acid residues indicate regions of conserved sequence (i.e., regions that may be required to preserve structural or functional properties), whereas non-highlighted amino acid residues are less conserved and can potentially be altered to a much broader extent without altering protein structure or function.

[image]

[image]

The claudins are a family of integral membrane proteins that are major components of tight junction (TJ) strands. When claudins are introduced into cells that lack tight junctions, networks of strands and grooves form at cell-cell contact sites that closely resemble native tight junctions. There are at least 17 members of this family in mammals. Claudin family members share ˜38% amino acid identity, and are predicted to have four transmembrane (TM) domains, which is reminiscent of occludin, although they share no sequence similarity with it. Multiple sequence alignment reveals their sequences to be fairly well conserved in the first and fourth putative TM domains, and in the first and second extracellular loops, but they diverge in the second and third TM domains. Although the sequences of their C-terminal cytoplasmic domains vary, the known family members share a common motif of -Y-V. This has been postulated as a possible binding motif for PDZ domains of other tight junction-associated peripheral membrane proteins, such as ZO-1.

The disclosed NOV1 nucleic acid of the invention encoding a Human Claudin-like protein includes the nucleic acid whose sequence is provided in Table 1A, 1C, 1E, 1G, or a fragment thereof. The invention also includes a mutant or variant nucleic acid any, of whose bases may be changed from the corresponding base shown in Table 1A, 1C, 1E, or 1G while still encoding a protein that maintains its Human Claudin-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 37 percent of the bases may be so changed.

The disclosed NOV1 protein of the invention includes the Human Claudin-like protein whose sequence is provided in Table 1B, 1D, 1F, or 1H. The invention also includes a mutant or variant protein any of whose residues may be changed from the corresponding residue shown in Table 1B, 1D, 1F, or 1H while still encoding a protein that maintains its Human Claudin-like activities and physiological functions, or a functional fragment thereof. In the mutant or variant protein, up to about 66 percent of the residues may be so changed.

The invention further encompasses antibodies and antibody fragments, such as F_{ab} or (F_{ab})₂, that bind immunospecifically to any of the proteins of the invention.

The above disclosed information suggests that this Human Claudin-like protein (NOV1) is a member of a “Human Claudin family”. Therefore, the NOV1 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV1 nucleic acids and proteins of the invention are useful in potential therapeutic applications implicated in cancer including but not limited to various pathologies and disorders as indicated below. For example, a cDNA encoding the Human Claudin-like protein (NOV1) may be useful in gene therapy, and the Human Claudin-like protein (NOV1) may be useful when administered to a subject in need thereof. By way of nonlimiting example, the compositions of the present invention will have efficacy for treatment of patients suffering from Von Hippel-Lindau (VHL) syndrome, Cirrhosis, Transplantation, Hemophilia, hypercoagulation, Idiopathic thrombocytopenic purpura, autoimmume disease, allergies, immunodeficiencies, transplantation, Graft vesus host, Alzheimer's disease, Stroke, Tuberous sclerosis, hypercalceimia, Parkinson's disease, Huntington's disease, Cerebral palsy, Epilepsy, Lesc\acute{h}-Nyhan syndrome, Multiple sclerosis, Ataxia-telangiectasia, Leukodystrophies, Behavioral disorders, Addiction, Anxiety, Pain, Neuroprotection, Systemic lupus erythematosus, Autoimmune disease, Asthma, Emphysema, Scleroderma, allergy, and Cancer, or other pathologies or conditions. The NOV1 nucleic acid encoding the Human Claudin-like protein of the invention, or fragments thereof, may further be useful in diagnostic applications, wherein the presence or amount of the nucleic acid or the protein are to be assessed.

NOV1 nucleic acids and polypeptides are further useful in the generation of antibodies that bind immuno-specifically to the novel NOV1 substances for use in therapeutic or diagnostic methods. These antibodies may be generated according to methods known in the art, using prediction from hydrophobicity charts, as described in the “Anti-NOVX Antibodies” section below. The disclosed NOV1 proteins have multiple hydrophilic regions, each of which can be used as an immunogen. These novel proteins can be used in assay systems for functional analysis of various human disorders, which will help in understanding of pathology of the disease and development of new drug targets for various disorders.

NOV2

A disclosed NOV2 nucleic acid of 1361 nucleotides (also referred to as CG56596-01) encoding a novel Protein Serine Kinase-like protein is shown in Table 2A. An open reading frame was identified beginning with an ATG initiation codon at nucleotides 20-22 and ending with a TAA codon at nucleotides 1268-1270. A The start and stop codons are in bold letters in Table 2A.

TABLE 2A
NOV2 nucleotide sequence.

(SEQ ID NO:9)

CGGCGGGCGTGTTGCGGGATGGGGTGCGGCGCCAGCAGGAAGGTGGTCCCGGGGCCACCAAAAATTCTTGT
AATAGAATTGGCATCCAAAGTGGAACCCAGAAATGGAACAAAGAATGATCTCTATAAATTTTTTTATTATAC
TTTAAGTTCTACTCCTCCCTGCCCTCTGCCACTCCCCTCACTACCCCAGTGCCCCCTCCCTCCTTGCCCTGG
GCCCGAGGCGGCGGCCCAGGCGGCGCAGAGGATACAGGTGGCTCGCTTCCGAGCCAAGTTCGACCCCCGGGT
CCTTGCCAGATATGACATCAAAGCTCTTATTGGGACAGGCAGTTTCAGCAGGGTTGTCAGGGTAGAGCAGAA
GACCACCAAGAAACCTTTTGCAATAAAAGTGATGGAAACCAGAGAGAGGGAAGGTAGAGAAGCGTGCGTGTC
TGAGCTGAGCGTCCTGCGGCGGGTTAGCCATCGTTACATTGTCCAGCTCATGGAGATCTTTGAGACTGAGGA
TCAAGTTTACATGGTAATGGAGCTGGCTACCGGAGGGGAGCTCTTTGATCGACTCATTGCTCAGGGATCCTT
TACAGAGCGGGATGCCGTCAGGATCCTCCAGATGGTTGCTGATGGGATTAGGTATTTGCATGCGCTGCAGAT
AACTCATAGGAATCTAAAGCCTGAAAACCTCTTATACTATCATCCAGGTGAAGAGTCGAAAATTTTAATTAC
AGATTTTGGTTTGGCATACTCCGGGAAAAAAAGTGGTGACTGGACAATGAAGACACTCTGTGGGACCCCAGA
GTACATAGCTCCTGAGGTTTTGCTAAGGAAGCCTTATACCAGTGCAGTGGACATGTGGGCTCTTGGTGTGAT
CACATATGCTTTACTTAGCGGATTCCTGCCTTTTGATGATGAAAGCCAGACAAGGCTTTACAGGAAGATTCT
GAAAGGCAAATATAATTATACAGGAGAGCCTTGGCCAAGCATTTCCCACTTGGCGAAGGACTTTATAGACAA
ACTACTGATTTTGGAGGCTGGTCATCGCATGTCAGCTGGCCAGGCCCTGGACCATCCCTGGGTGATCACCAT
GGCTGCAGGGTCTTCCATGAAGAATCTCCAGAGGGCCATATCCCGAAACCTCATGCAGAGGGCCTCTCCCCA
CTCTCAGAGTCCTGGATCTGCACAGTCTTCTAAGTCACATTATTCTCACAAATCCAGGCATATGTGGAGCAA
GAGAAACTTAAGGATAGTAGAATCGCCACTGTCTGCGCTTTTGTAAGCAGATGACCTCTAAAACTATTTTTG
CCTATTTTAGGACCATTTCATCATGATTAGGGCACCCTCAAGCTCCAAAGACACGGGACTCCATG

The disclosed NOV2 nucleic acid sequence, localized to the q21.3-22 region of chromsome 18, has 685 of 997 bases (68%) identical to a gb:GENBANK-ID:HSA272212|acc:AJ272212.1 mRNA from Homo sapiens (mRNA for protein serine kinase (PSKH1 gene)) (E=6.1e⁻⁸⁵).

A NOV2 polypeptide (SEQ ID NO:10) encoded by SEQ ID NO:9 has 416 amino acid residues and is presented using the one-letter code in Table 2B. Signal P, Psort and/or Hydropathy results predict that NOV2 contains no signal peptide and is likely to be localized to the endoplasmic reticulum (membrane) with a certainty of 0.5500. Alternatively, NOV2 20 may also localize to the lysosome (lumen) with a certainty of 0.2403, the plasma membrane with a certainty of 0.1900, or the microbody (peroxisome) with a certainty of 0.1111.

TABLE 2B
Encoded NOV2 protein sequence.

(SEQ ID NO:10)

MGCGASRKVVPGPPKILVIELASKVEPRNGTKNDLYKFFYYTLSSTPPCPLPLPSLPQCPLPPCPGPEAAAQ
AAQRIQVARFRAKFDPRVLARYDIKALIGTGSFSRVVRVEQKTTKKPFAIKVMETREREGREACVSELSVLR
RVSHRYIVQLMEIFETEDQVYMVMELATGGELFDRLIAQGSFTERDAVRILQMVADGIRYLHALQITHRNLK
PENLLYYHPGEESKILITDFGLAYSGKKSGDWTMKTLCGTPEYIAPEVLLRKPYTSAVDMWALGVITYALLS
GFLPFDDESQTRLYRKILKGKYNYTGEPWPSISHLAKDFIDKLLILEAGHRMSAGQALDHPWVITMAAGSSM
KNLQRAISRNLMQRASPHSQSPGSAQSSKSHYSHKSRHMWSKRNLRIVESPLSALL

The disclosed NOV2 amino acid sequence has 267 of 412 amino acid residues (64%) identical to, and 332 of 412 amino acid residues (80%) similar to, the 424 amino acid residue ptnr:SPTREMBL-ACC:Q9NY19 protein from Homo sapiens (Human) (Protein Serine Kinase) (E=1.1e⁻¹³⁸).

NOV2 is predicted to be expressed in Kidney, Lymph node, Pancreas, Salivary Glands, Brain, and Placenta because of the expression pattern of (GENBANK-ID: gb:GENBANK-ID:HSA272212|acc:AJ272212.1) a closely related Homo sapiens mRNA for protein serine kinase (PSKH1 gene) homolog.

In addition, the sequence is predicted to be expressed in keratinocytes because of the expression pattern of (GENBANK-ID: gb:GENBANK-ID:HSPI13711|acc:AJ001696.2) a closely related Homo sapiens mRNA for hurpin, clone R7-1.1 homolog.

NOV2 also has homology to the amino acid sequences shown in the BLASTP data listed in Table 2C.

TABLE 2C
BLAST results for NOV2

| Gene Index/ | Protein/ | Length | Identity | Positives | |
|---|---|---|---|---|---|
| Identifier | Organism | (aa) | (%) | (%) | Expect |
| gi\|14916455\|ref\|NP_{—} | serine/threonine | 385 | 369/416 | 372/416 | 0.0 |
| 149117.1\| | kinase | | (88%) | (88%) | |
| (NM_033126) | PSKH2 [Homo | | | | |
| | sapiens] | | | | |
| gi\|17530179\|gb\|AAL40735.1\| | protein | 975 | 257/391 | 318/391 | e−149 |
| (AF416988) | serine | | (65%) | (80%) | |
| | kinase/luciferase | | | | |
| | fusion | | | | |
| | protein | | | | |
| gi\|14776113\|ref\|XP_{—} | hypothetical | 424 | 257/391 | 318/391 | e−145 |
| 043047.1\| | protein | | (65%) | (80%) | |
| (XM_043047) | XP_043047 | | | | |
| | [Homo | | | | |
| | sapiens] | | | | |
| gi\|15963448\|gb\|AAL11033.1\| | protein | 424 | 254/386 | 311/386 | e−144 |
| (AF236367) | serine | | (65%) | (79%) | |
| | kinase Pskh1 | | | | |
| | [Mus | | | | |
| | musculus] | | | | |
| gi\|2136035\|pir\|\|I38138 | protein- | 319 | 209/320 | 258/320 | e−115 |
| | serine | | (65%) | (80%) | |
| | kinase (EC | | | | |
| | 2.7.1.—) | | | | |
| | PSK-H1 - | | | | |
| | human | | | | |
| | (fragment) | | | | |

The homology of these sequences is shown graphically in the ClustalW analysis shown in Table 2D.

[image]

[image]

[image]

The presence of identifiable domains in NOV2, as well as all other NOVX proteins, was determined by searches using software algorithms such as PROSITE, DOMAIN, Blocks, Pfam, ProDomain, and Prints, and then determining the Interpro number by crossing the domain match (or numbers) using the Interpro website (http:www.ebi.ac.uk/interpro). DOMAIN results for NOV2 as disclosed in Tables 2E-2G, were collected from the Conserved Domain Database (CDD) with Reverse Position Specific BLAST analyses. This BLAST analysis software samples domains found in the Smart and Pfam collections. For Table 2K and all successive DOMAIN sequence alignments, fully conserved single residues are indicated by black shading or by the sign (|) and “strong” semi-conserved residues are indicated by grey shading or by the sign (+). The “strong” group of conserved amino acid residues may be any one of the following groups of amino acids: STA, NEQK, NHQK, NDEQ, QHRK, MILV, MILF, HY, FYW.

Tables 2E-G lists the domain description from DOMAIN analysis results against NOV2. This indicates that the NOV2 sequence has properties similar to those of other proteins known to contain this domain.

TABLE 2E
Domain Analysis of NOV2

gnl|Smart|smart00220, S_TKc, Serine/Threonine protein kinases,
catalytic domain; Phosphotransferases. Serine or threonine-specific
kinase subfamily. (SEQ ID NO:799)
CD-Length = 256 residues, 100.0% aligned
Score = 261 bits (668), Expect = 4e−71

| | | | |
|---|---|---|---|
| NOV 2: | 94 | YDIKALIGTGSFSRVVRVEQKTTKKPFAIKVMETRE--REGREACVSELSVLRRVSHRYI | 151 |
| | | \|++ ++\| \|+\| +\| \| \| \| \|\|\|\|++ + ++ \|\| + \|+ +\|+++ \| \| | |
| Sbjct: | 1 | YELLEVLGKCAFGKVYLARDKKTGKLVAIKVIKKEKLKKKKRERILREIKILKKLDHPNI | 60 |
| NOV2: | 152 | VQLMEIFETEDQVYMVMELATOCELFDRLIAQGSFTERDAVRILQMVADCIRYLHALQIT | 211 |
| | | \|+\| ++\|\| +\|++\|+\|\|\| \|\|+\|\|\| \| +\| +\| +\| + + + \|\|\|+ \| | |
| Sbjct: | 61 | VKLYDVFEDDDKLYLVMEYCEGGDLFDLLKKRGRLSEDEARFYARQILSALEYLHSQGII | 120 |
| NOV 2: | 212 | HRNLKPENLLYYHPGEESKILITDFGLAYSCKKSGDWTMKTLCGTPEYIAPEVLLRKPYT | 271 |
| | | \|\|+\|\|\|\|\|+\| \| + + \|\|\|\|\| \|\| + \| \|\|\|\|\|+\|\|\|\|\|\| \| \| | |
| Sbjct: | 121 | HRDLKPENILLDSDGH---VKLADFQLA-KQLDSGGTLLTTFVGTPEYMAPEVLLGKGYG | 176 |
| NOV 2: | 272 | SAVDMWALGVITYALLSCFLPFDDESQTRLYRKILKGKYNYTGEPWPSISHLAKDFIDKL | 331 |
| | | \|\|\|+\|+\|\|\|\| \| \|\|+\| \|\| + \| \| + \| \|\| \|\|\| \| \|\| | |
| Sbjct: | 177 | KAVDIWSLGVILYELLTGKPPFPGDDQLLALFKKIGKPPPPFPPPEWKISPEAKDLIKKL | 236 |
| NOV 2: | 332 | LILEAGHRMSAGQALDHPWV 351 | |
| | | \|+ + \|++\| +\|\|+\|\|+ | |
| Sbjct: | 237 | LVKDPEKRLTAEEALEHPFF 256 | |

TABLE 2F
Domain Analysis of NOV2

gnl|Pfam|pfam00069, pkinase, Protein kinase domain (SEQ ID NO:800)
CD-Length = 256 residues, 100.0% aligned
Score = 230 bits (586), Expect = 1e−61

| | | | |
|---|---|---|---|
| NOV 2: | 94 | YDIKALIOTGSFSRVVRVEQKTTKKPFAIKVMETREREGREACV-SELSVLRRVSHRYIV | 152 |
| | | \|++ +\|+\|+\| +\| + + \| \| + \|\|\|+++ \| ++ \|+ +\|\|\|+\|\| \|\| | |
| Sbjct: | 1 | YELGEKLGSGAEGKVYKGKHKDTGEIVAIKILKKRSLSEKKKRFLREIQILRRLSHPNIV | 60 |
| NOV2: | 153 | QLMEIFETEDQVYMVNELATOGELFDRLIAQGS-FTERDAVRILQMVADGIRYLHALQIT | 211 |
| | | +\|+ +\|\| +\| +\|+\|\|\| \|\|+\|\|\| \| \| +\|++\| +\| + \|+ \|\|\|+ \| | |
| Sbjct: | 61 | RLLGVFEEDDHLYLVMEYMEGGDLFDYLRRNGLLLSEKEAKKIALQILRGLEYLHSRCIV | 120 |
| NOV 2: | 212 | HRNLKPENLLYYHPGEESKILITDFGLAYSGKKSGDWVMKVLCGTPEYIAPEVLLRKPYT | 271 |
| | | \|\|+\|\|\|\|\|+\| \| + \| \|\|\|\|\| + \| + \| \|\|\|\|\|+\|\|\|\|\| + \|+ | |
| Sbjct: | 121 | HRDLKPENILLDENGT---VKIADFGLARKLESSSYEKLVVFVGTPEYMAPEVLEGRGYS | 177 |
| NOV 2: | 272 | SAVDMWALGVITYALLSGFLPFDDESQVRLYRKILKGKYNYTGEPWPSISHLAKDFIDKL | 331 |
| | | \| \|\|+\|+\|\|\|\| \| \|\|+\| \|\|\| +\| + \|+ \| \|\| \| \| | |
| Sbjct: | 178 | SKVDVWSLGVILYELLTGKLPFPGIDPLEELFRIKERPR-LRLPLPPNCSEELKDLIKKC | 236 |
| NOV 2: | 332 | LILEAGHRMSAGQALDHPNV 351 | |
| | | \| + \| +\| + \|+\|\|\| | |
| Sbjct: | 237 | LNKDPEKRPTAKEILNHPWF 256 | |

TABLE 2G
Domain Analysis of NOV2

gnl|Smart|smart00219, TyrKc, Tyrosine kinase, catalytic domain;
Phosphotransferases. Tyrosine-specific kinase subfamily. (SEQ ID
NO: 801)
CD-Length = 258 residues, 837% aligned
Score = 117 bits (292), Expect = 2e−27

| | | | |
|---|---|---|---|
| NOV 2: | 100 | IGTGSFSRVVR---VEQKTTKKPFAIKVM-ETREREGREACVSELSVLRRVSHRYIVQLM | 155 |
| | | +\| \|+\| \| + + + \|+\| + \| + \| + \| ++\|++ \| \|\|+\|+ | |
| Sbjct: | 7 | LGEGAFCEVYKGTLKGKGGVEVEVAVKTLKEDASEQQIEEFLREARLMRKLDHPNIVKLL | 66 |
| NOV 2: | 156 | EIFETEDQVYMVMELATGGELFDRLIAQG--SFTERDAVRILQMVADGIRYLHALQITHR | 213 |
| | | + \|+ + +\|\|\| \|\|+\| \| \| + \| + +\| \|+ \|\| + \|\| | |
| Sbjct: | 67 | GVCTEEEPLMIVMEYMEGGDLLDYLRKNRPKELSLSDLLSFALQIARGMEYLESKNFVHR | 126 |
| NOV 2: | 214 | NLKPENLLYYHPGEESKILITDFGLAYSGKKSGDWTMKTLCGTP-EYIAPEVLLRKPYTS | 272 |
| | | +\| \| \| \|\| + \| \|\|\|\|\| + \| \| \| ++\|\|\| \| +\|\| | |
| Sbjct: | 127 | DLAARNCLV---GENKTVKIADFGLARDLYDDDYYRKKKSPRLPIRWMAPESLKDGKFTS | 183 |
| NOV2: | 273 | AVDMWALGVITYALLS-GFLPFDDESQTRLYRKILKGKY 310 | |
| | | \|+\|+ \|\|+ + + + \| \|+ \| + + \|\| | |
| Sbjct: | 184 | KSDVWSFGVLLWEIFTLGESPYPGMSNEEVLEYLKKGYR 222 | |

Protein phosphorylation is a fundamental process for the regulation of cellular functions. The coordinated action of both protein kinases and phosphatases controls the levels of phosphorylation and, hence, the activity of specific target proteins. One of the predominant roles of protein phosphorylation is in signal transduction, where extracellular signals are amplified and propagated by a cascade of protein phosphorylation and dephosphorylation events. Eukaryotic protein kinases are enzymes that belong to a very extensive family of proteins which share a conserved catalytic core common with both serine/threonine and tyrosine protein kinases. There are a number of conserved regions in the catalytic domain of protein kinases. In the N-terminal extremity of the catalytic domain there is a glycine-rich stretch of residues in the vicinity of a lysine residue, which has been shown to be involved in ATP binding. In the central part of the catalytic domain there is a conserved aspartic acid residue which is important for the catalytic activity of the enzyme.

The disclosed NOV2 nucleic acid of the invention encoding a Protein Serine Kinase-like protein includes the nucleic acid whose sequence is provided in Tables 2A or a fragment thereof. The invention also includes a mutant or variant nucleic acid any of whose bases may be changed from the corresponding base shown in Tables 2A while still encoding a protein that maintains its Protein Serine Kinase-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 32 percent of the bases may be so changed.

The disclosed NOV2 protein of the invention includes the Protein Serine Kinase-like protein whose sequence is provided in Tables 2B. The invention also includes a mutant or variant protein any of whose residues may be changed from the corresponding residue shown in Table 2B while still encoding a protein that maintains its Protein Serine Kinase-like activities and physiological functions, or a functional fragment thereof. In the mutant or variant protein, up to about 35 percent of the residues may be so changed.

The NOV2 nucleic acids and proteins of the invention are useful in potential therapeutic applications implicated in Diabetes, Von Hippel-Lindau (VHL) syndrome, Pancreatitis, Obesity, Lymphedema, Allergies, Alzheimer's disease, Stroke, Tuberous sclerosis, hypercalceimia, Parkinson's disease, Huntington's disease, Cerebral palsy, Epilepsy, Lesch-Nyhan syndrome, Multiple sclerosis, Ataxia-telangiectasia, Leukodystrophies, Behavioral disorders, Addiction, Anxiety, Pain, Neuroprotection, Diabetes, Autoimmune disease, Renal artery stenosis, Interstitial nephritis, Glomerulonephritis, Polycystic kidney disease, Systemic lupus erythematosus, Renal tubular acidosis, IgA nephropathy, and/or other pathologies and disorders.

NOV2 nucleic acids and polypeptides are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. The disclosed NOV2 protein has multiple hydrophilic regions, each of which can be used as an immunogen. These novel proteins can be used in assay systems for functional analysis of various human disorders, which are useful in understanding of pathology of the disease and development of new drug targets for various disorders.

NOV3

NOV3 includes three novel human 1 Claudin-like proteins disclosed below. The disclosed sequences have been named NOV3a, NOV3b, and NOV3c.

NOV3a

A disclosed NOV3a nucleic acid of 695 nucleotides (designated CuraGen Acc. No. CG56594-01) encoding a novel Claudin-19-like protein is shown in Table 3A. An open reading frame was identified beginning with an ATG initiation codon at nucleotides 53-55 and ending with a TGA codon at nucleotides 662-664. A putative untranslated region downstream from the termination codon is underlined in Table 3A, and the start and stop codons are in bold letters.

TABLE 3A
NOV3a Nucleotide Sequence

(SEQ ID NO:11)

GCACCCTGGCCCAGCTCTGAGTCCTGGGACCCTCGGTCCTCTCTCCTGGGCCATGGCCAACTCAGGCCTC
CAGCTCCTGGGCTACTTCTTGGCCCTGGGTGGCTGGGTGGGCATCATTGCTAGCACAGCCCTGCCACAGT
GGAAGCAGTCTTCCTACGCAGGCGACGCCATCATCACTGCCGTGGGCCTCTATGAAGGGCTCTGGATGTC
CTGCGCCTCCCAGAGCACTGGGCAAGTGCAGTGCAAGCTCTACGACTCGCTGCTCGCCCTGGACGGTAGG
CCCCAGGCCGCGCGGGCCCTGATGGTGGTGGCCGTGCTCCTGGGCTTCGTGGCCATGGTCCTCAGCGTAG
TTGGCATGAAGTGTACGCGGGTGGGAGACAGCAACCCCATTGCCAAGGGCCGTGTTGCCATCGCCGGGGG
AGCCCTCTTCATCCTGGCAGGCCTCTGCACTTTGACTGCTGTCTCGTGGTATGCCACCCTGGTGACCCAG
GAGTTCTTCAACCCAGAATTTGGCCCAGCCCTGTTCGTGGGCTGGGCCTCAGCTGGCCTGGCCGTGCTGG
GCGGCTCCTTCCTCTGCTGCACATGCCCGGAGCCAGAGAGACCCAACAGCAGCCCACAGCCCTATCGGCC
TGGACCCTCTGCTGCTGCCCGAGAGTACGTCTGAGCTCCGCCTGCCCTGGCCAGCCCCCCACCCA

The nucleic acid sequence, localized to chromosome 1, has 402 of 482 bases (83%) identical to a gb:GENBANK-ID:AF249889|acc:AF249889.1 mRNA from Mus musculus (claudin-19 mRNA, partial cds) (E=1.1e⁻⁶⁷).

A NOV3a polypeptide (SEQ ID NO:12) encoded by SEQ ID NO:11 is 203 amino acid residues and is presented using the one letter code in Table 3B. Signal P, Psort and/or Hydropathy results predict that NOV3a has no signal peptide and is likely to be localized at the endoplasmic reticulum (membrane) with a certainty of 0.6850. Alternatively, NOV3a may also localize to the plasma membrane with a certainty of 0.6400, the Golgi body with a certainty of 0.4600, or the endoplasmic reticulum (lumen) with a certainty of 0.1000. The most likely cleavage site for NOV3a is between positions 23 and 24: IIA-ST.

TABLE 3B
NOV3a protein sequence

(SEQ ID NO:12)

MANSGLQLLGYFLALGGWVGIIASTALPQWKQSSYAGSAIITAVGLYEGLWMSCASQSTGQVQCKLYDSLLALD
GRPQAARALMVVAVLLGFVAMVLSVVGMKCTRVGDSNPIAKGRAVIAGGALFILAGLCTLTAVSWYATLVTQEF
FNPEFGPALFVGWASAGLAVLGGFLCCTCPEPERPNSSPQPYRPGPSAAAREYV

The full amino acid sequence of the protein of the invention was found to have 174 of 193 amino acid residues (90%) identical to, and 178 of 193 amino acid residues (92%) similar to, the 193 amino acid residue ptnr:TREMBLNEW-ACC:AAF98323 protein from Mus musculus (Mouse) (CLAUDIN-19) (E=5.7e⁻⁸⁹).

NOV3a is predicted to be expressed in at least the Spinal cord.

NOV3b

A disclosed NOV3b nucleic acid of 695 nucleotides (also referred to as CG56594-01) encoding a novel Claudin-19-like protein is shown in Table 3C. An open reading frame was identified beginning with an ATG initiation codon at nucleotides 53-55 and ending with a TGA termination codon at nucleotides 662-664. The start and stop codons are in bold letters in Table 3C, and the 5′ and 3′ untranslated regions are underlined.

TABLE 3C
NOV3b nucleotide sequence.

(SEQ ID NO:13)

GCACCCTGGCCCAGCTCTGAGTCCTGGGACCCTCGGTCCTCTCTCCTGGGCCATGGCCAACTCAGGCCTCCA
GCTCCTGGGCTACTTCTTGGCCCTGGGTGGCTGGGTGGGCATCATTGCTAGCACAGCCCTGCCACAGTGGAA
GCAGTCTTCCTACGCAGGCGACGCCATCATCACTGCCGTGGGCCTCTATGAAGGGCTCTGGATGTCCTGCGC
CTCCCAGAGCACTGGGCAAGTGCAGTGCAAGCTCTACGACTCGCTGCTCGCCCTGGACGGTAGGCCCCAGGC
CGCGCGGGCCCTGATGGTGGTGGCCGTGCTCCTGGGCTTCGTGGCCATGGTCCTCAGCGTAGTTGGCATGAA
GTGTACGCGGGTGGGAGACAGCAACCCCATTGCCAAGGGCCGTGTTGCCATCGCCGGGGGAGCCCTCTTCAT
CCTGGCAGGCCTCTGCACTTTGACTGCTGTCTCGTGGTATGCCACCCTGGTGACCCAGGAGTTCTTCAACCC
AGAATTTGGCCCAGCCCGTTCGTGGGCTGGGCCTCAGCTGGCCTGGCCGTGCTGGGCGGCTCCTTCCTCTG
CTGCACATGCCCGGAGCCAGAGAGACCCAACAGCAGCCCACAGCCCTATCGGCCTGGACCCTCTGCTGCTGC
CCGAGAGTACGTCTGAGCTCCGCCTGCCCTGGCCAGCCCCCCACCCA

In a search of public sequence databases, the NOV3b nucleic acid sequence, located on chromsome 1 has 402 of 482 bases (83%) identical to a gb:GENBANK-ID:AF249889|acc:AF249889.1 mRNA from Mus musculus (claudin-19 mRNA, partial cds) (E=1.1e⁻⁶⁷).

The disclosed NOV3b polypeptide (SEQ ID NO:14) encoded by SEQ ID NO:13 has 203 amino acid residues and is presented in Table 3D using the one-letter amino acid code. Signal P, Psort and/or Hydropathy results predict that NOV3b has a signal peptide and is likely to be localized the endoplasmic reticulum (membrane) with a certainty of 0.6850. Alternatively, NOV3b may also localize to the plasma membrane with acertainty of 0.6400, the Golgi body with a certainty of 0.4600 or in the endoplasmic reticulum (lumen) with a certainty of 0.1000. The most likely cleavage site for NOV3b is between positions 23 and 24: IIA-ST.

TABLE 3D
Encoded NOV3b protein sequence.

(SEQ ID NO:14)

MANSGLQLLGYFLALGGWVGIIASTALPQWKQSSYAGDAIITAVGLYEGLWMSCASQSTGQVQCKLYDSLLA
LDGRPQAARALMVVAVLLGFVAMVLSVVGMKCTRVGDSNPIAKGRVAIAGGALFILAGLCTLTAVSWYATLV
TQEFFNPEFGPALFVGWASAGLAVLGGSFLCCTCPEPERPNSSPQPYRPGPSAAAREYV

A search of sequence databases reveals that the NOV3b amino acid sequence has 174 of 193 amino acid residues (90%) identical to, and 178 of 193 amino acid residues (92%) similar to, the 193 amino acid residue ptnr:TREMBLNEW-ACC:AAF98323 protein from Mus musculus (Mouse) (Claudin-19) (E=5.7e⁻⁸⁹).

NOV3b is predicted to be expressed in at least the Spinal cord.

NOV3c

A disclosed NOV3c nucleic acid of 690 nucleotides (also referred to as CG57576-01) encoding a novel Claudin 19-like protein is shown in Table 3E. An open reading frame was identified beginning with an ATG codon at nucleotides 51-53 and ending with a TGA codon at nucleotides 684-686. The start and stop codons are in bold letters and the 5′ and 3′ untranslated regions are underlined in Table 3I. Because the start codon is not a traditional initiation codon, NOV3c could be a partial reading frame. NOV3c could extend further in the 5′ direction.

TABLE 3E
NOV3c nucleotide sequence.

(SEQ ID NO:15)

ACCCTGGCCCAGCTCTGAGTCCTGGGACCCTCGGTCCTCTCTCCTGGCCATGGCCAACTCAGGCCTCCAGC
TCCTGGGCTACTTCTTGGCCCTGGGTGGCTGGGTGGGCATCATTGCTAGCACAGCCCTGCCACAGTGGAAGC
AGTCTTCCTACGCAGGCGACGCCATCATCACTGCCGTGGGCCTCTATGAAGGGCTCTGGATGTCCTGCGCCT
CCCAGAGCACTGGGCAAGTGCAGTGCAAGCTCTACGACTCGCTGCTCGCCCTGGACGGTCACATCCAATCAG
CGCGGGCCCTGATGGTGGTGGCCGTGCTCCTGGGCTTCGTGGCCATGGTCCTCAGCGTAGTTGGCATGAAGT
GTACGCGGGTGGGAGACAGCAACCCATTGCCAAGGGCCGTGTTGCCATCGCCGGGGGAGCCCTCTTCATCC
TGGCAGGCCTCTGCACTTTGACTGCTGTCTCGTGGTATGCCACCCTGGTGACCCAGGAGTTCTTCAACCCAA
GCACACCTGTCAATGCCAGGTATGAATTTGGCCCAGCCCTGTTCGTGGGCTGGGCCTCAGCTGGCCTGGCCG
TGCTGGGCGGCTCCTTCCTCTGCTGCACATGCCCGGAGCCAGAGAGACCCAACAGCAGCCCACAGCCCTATC
GGCCTGGACCCTCTGCTGCTGCCCGAGAGTACGTCTGAGCTC

In a search of public sequence databases, the NOV3c nucleic acid sequence, located on chromsome 1 has 445 of 671 bases (66%) identical to a gb:GENBANK-ID:HSA011497|acc:AJ011497.1 mRNA from Homo sapiens (mRNA for Claudin-7) (E=5.3e⁻⁴⁶).

The disclosed NOV3c polypeptide (SEQ ID NO:16) encoded by SEQ ID NO:15 has 211 amino acid residues and is presented in Table 3F using the one-letter amino acid code. Signal P, Psort and/or Hydropathy results predict that NOV3c has no signal peptide and is likely to be localized the the endoplasmic reticulum (membrane) with a certainty of 0.6850. Alternatively, NOV3c may also localize to the plasma membrane with acertainty of 0.6400, the Golgi body with a certainty of 0.4600 or in the endoplasmic reticulum (lumen) with a certainty of 0.1000. The most likely cleavage site for a NOV3c peptide is between amino acids 23 and 24, at: IIA-ST.

TABLE 3F
Encoded NOV3c protein seqnence.

(SEQ ID NO:16)

MANSGLQLLGYFLALGGWVGIIASTALPQWKQSSYAGDAIITAVGLYEGLWMSCASQSTGQVQCKLYDSLLA
LDGHIQSARALMVVAVLLGFVAMVLSVVGMKCTRVGDSNPIAKGRVAIAGGALFILAGLCTLTAVSWYATLV
TQEFFNPSTPVNARYEFGPALFVGWASAGLAVLGGSFLCCTCPEPERPNSSPQPYRPGPSAAAREYV

A search of sequence databases reveals that the NOV3c amino acid sequence has 121 of 211 amino acid residues (57%) identical to, and 159 of 211 amino acid residues (75%) similar to, the 211 amino acid residue ptnr:SWISSNEW-ACC:O95832 protein from Homo sapiens (Human) (Claudin-1 (Senescence-Associated Epithelial Membrane Protein)) (E=9.6e⁻⁶⁶).

NOV3c is predicted to be expressed in at least Spinal cord.

NOV3a also has homology to the amino acid sequences shown in the BLASTP data listed in Table 3G.

TABLE 3G
BLAST results for NOV3a

| Gene Index/ | | Length | Identity | Positives | |
|---|---|---|---|---|---|
| Identifier | Protein/Organism | (aa) | (%) | (%) | Expect |
| gi\|9789476\|gb\|AAF98323.1\| | claudin-19 [Mus | 193 | 174/193 | 178/193 | 1e−84 |
| (AF249889) | musculus] | | (90%) | (92%) | |
| gi\|17489134\|ref\|XP_{—} | similar to | 309 | 126/137 | 127/137 | 3e−59 |
| 060892.1\| | claudin-19 (H. | | (91%) | (91%) | |
| (XM_060892) | sapiens) [Homo | | | | |
| | sapiens] | | | | |
| gi\|12654455\|gb\|AAH01055.1\| | claudin 7 [Homo | 211 | 112/211 | 149/211 | 2e−55 |
| AAH01055 | sapiens] | | (53%) | (70%) | |
| (BC001055) | | | | | |
| gi\|10835008\|ref\|NP_{—} | claudin 7; | 211 | 111/211 | 148/211 | 7e−55 |
| 001298.1\| | Clostridium | | (52%) | (69%) | |
| (NM_001307) | perfringens | | | | |
| | enterotoxin | | | | |
| | receptor-like 2; | | | | |
| | claudin 9 [Homo | | | | |
| | sapiens] | | | | |
| gi\|7710002\|ref\|NP_057883.1\| | claudin 1 [Mus | 211 | 112/212 | 149/212 | 8e−55 |
| (NM_016674) | musculus] | | (52%) | (69%) | |

The homology of these sequences is shown graphically in the ClustalW analysis shown in Table 3H.

[image]

[image]

Table 3I lists the domain description from DOMAIN analysis results against NOV3. This indicates that the NOV3 sequence has properties similar to those of other proteins known to contain this domain.

TABLE 31
Domain Analysis of NOV3

gnl|Pfam|pfam00822, PMP22_Claudin, PMP-22/EMP/MP20/Claudin family
(SEQ ID NO:802)
CD-Length = 162 residues, 99.4% aligned
Score = 80.5 bits (197), Expect = 9e−17

| | | | |
|---|---|---|---|
| NOV 3: | 5 | GLQLLGYFLALGGWVG-IIASTALPQWKQSSYAGDAIITAVGLYEGLWMSCASQS-TQV | 62 |
| | | + \|\|\| + + + \|\| + + \| \|\|\| \| \| \| \| \|\|\| + \| + \|\| \|\|\| + | |
| Sbjct: | 2 | LVLLLGFIVSHIAWVILLFVATITDQWKVSRYVGAAA------SAGLWRNCTTQSCTGQI | 55 |
| NOV 3: | 63 | QCKLYDSLLALDGRPQAARALMVVAVLLGFVAMVLSVVGMKCTRVGDSNPIAKGRVAIAG | 122 |
| | | \|\| + \| \| + \|\| + \|\|\| + + + + + \|\| + + + + + + \| \| + \| | |
| Sbjct: | 56 | SCKV----LELNDALQAVQALMILSIILGIISLIVFFFQLFTMRKGGRFKLA-------- | 103 |
| NOV 3: | 123 | GALFILAGLCTLTAVSWYATLVTQEFFNP-------EFGPALFVGWASAGLAVLGGSFL | 174 |
| | | \| + \| + + + \|\|\| \| \| \| + + + \| \|\| \|\| + + \|\| + \|\| + \|\| | |
| Sbjct: | 104 | GIIFLVSGLCVLVGASIYTSRIATDFGNPFTPNRKYSFGYSFILGWVAFALAFIGGVLY | 162 |

The claudins are a family of integral membrane proteins that are major components of tight junction (TJ) strands. When claudins are introduced into cells that lack tight junctions, networks of strands and grooves form at cell-cell contact sites that closely resemble native tight junctions. There are at least 17 members of this family in mammals. Claudin family members share ˜38% amino acid identity, and are predicted to have four transmembrane (TM) domains, which is reminiscent of occludin, although they share no sequence similarity with it. Multiple sequence alignment reveals their sequences to be fairly well conserved in the first and fourth putative TM domains, and in the first and second extracellular loops, but they diverge in the second and third TM domains. Although the sequences of their C-terminal cytoplasmic domains vary, the known family members share a common motif of -Y-V. This has been postulated as a possible binding motif for PDZ domains of other tight junction-associated peripheral membrane proteins, such as ZO-1.

The disclosed NOV3 nucleic acid of the invention encoding a Claudin-19-like protein includes the nucleic acid whose sequence is provided in Table 3A or a fragment thereof. The invention also includes a mutant or variant nucleic acid any of whose bases may be changed from the corresponding base shown in Table 3A while still encoding a protein that maintains its Claudin-19-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 17 percent of the bases may be so changed.

The disclosed NOV3 protein of the invention includes the Claudin-19-like protein whose sequence is provided in Table 3B. The invention also includes a mutant or variant protein any of whose residues may be changed from the corresponding residue shown in Table 3B while still encoding a protein that maintains its Claudin-19-like activities and physiological functions, or a functional fragment thereof. In the mutant or variant protein, up to about 48 percent of the residues may be so changed.

The protein similarity information, expression pattern, and map location for the Claudin-19-like protein and nucleic acid (NOV3) disclosed herein suggest that this NOV3 protein may have important structural and/or physiological functions characteristic of the Claudin-19family. Therefore, the NOV3 nucleic acids and proteins of the invention are useful in potential diagnostic and therapeutic applications. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo.

The NOV3 nucleic acids and proteins of the invention are useful in potential diagnostic and therapeutic applications implicated in various diseases and disorders described below. For example, the compositions of the present invention will have efficacy for treatment of patients suffering from Von Hippel-Lindau (VHL) syndrome, Cirrhosis, Transplantation, Hemophilia, hypercoagulation, Idiopathic thrombocytopenic purpura, autoimmume disease, allergies, immunodeficiencies, transplantation, Graft vesus host, Alzheimer's disease, Stroke, Tuberous sclerosis, hypercalceimia, Parkinson's disease, Huntington's disease, Cerebral palsy, Epilepsy, Lesch-Nyhan syndrome, Multiple sclerosis, Ataxia-telangiectasia, Leukodystrophies, Behavioral disorders, Addiction, Anxiety, Pain, Neuroprotection, Systemic lupus erythematosus, Autoimmune disease, Asthma, Emphysema, Scleroderma, allergy, and Cancer, and/or other pathologies. The NOV3 nucleic acids, or fragments thereof, may further be useful in diagnostic applications, wherein the presence or amount of the nucleic acid or the protein are to be assessed.

NOV3 nucleic acids and polypeptides are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. These novel proteins can be used in assay systems for functional analysis of various human disorders, which will help in understanding of pathology of the disease and development of new drug targets for various disorders.

NOV4

NOV4 includes three novel human 1 Claudin-like proteins disclosed below. The disclosed sequences have been named NOV4a, NOV4b, and NOV4c.

NOV4a

A disclosed NOV4a nucleic acid of 694 nucleotides (also referred to as CG56589-01) encoding a novel Claudin-6-like protein is shown in Table 4A. An open reading frame was identified beginning with an ATG initiation codon at nucleotides 11-13 and ending with a TAA codon at nucleotides 671-673. Putative untranslated regions upstream from the initiation codon and downstream from the termination codon are underlined in Table 4A, and the start and stop codons are in bold letters.

TABLE 4A
NOV4a Nucleotide Sequence

| | |
|---|---|
| ACCTGTCGCAATGGCTTTAATCTTTAGAACAGCAATGCAATCTGTTGGACTTTTACTATCTC | (SEQ ID NO:17) |
| TCCTGGGATGGATTTTATCCATTATTACAACTTATTTGCCACACTGGAAGAACCTCAACCTG | |
| GACTTAAATGAAATGGAAAACTGGACCATGGGACTCTGGCAAACCTGTGTCATCCAAGAGGA | |
| AGTCGGGATGCAATGCAAGGACTTTGACTCCTTCCTGGCTTTGCCTGCTGAACTCAGGGTCT | |
| CCAGGATCTTAATGTTTCTGTCAAATGGGCTGCGATTTCTGGGCCTGCTGGTCTCTGGGTTT | |
| GGCCTGGACTGTTTGAGAATTGGAGAGAGTCAGAGAGATCTCAAGAGGCGACTGCTCATTCT | |
| GGGAGGAATTCTGTCCTGGGCCTCGGGAATCACAGCCCTGCTTCCCGTCTCTTGGGTTGCCC | |
| ACAAGACGGTTCAGGAGTTCTGGGATGAQAACGTCCCAGACTTTGTCCCCAGGTGGGAGTTT | |
| GGGGAGGCCCTGTTTCTGGGCTGGTTTGCTGGACTTTCTCTTCTGCTAGGAGGGTGTCTGCT | |
| CAACTGCGCAGCCTCCTCCAGCCACGCTCCCCTAGCTTTGGGCCACTATGCAGTGGCGCAAA | |
| TGCAAACTCAGTGTCCCTACCTGGAAGATGGGACAGCAGATCCTCAAGTGTAAGACTCCGAC | |
| AAGGCCAGAGAT | |

The NOV4a nucleic acid was identified on chromosome 4 and has 330 of 556 bases (59%) identical to a gb:GENBANK-ID:AF134160|acc:AF134160.1 mRNA from Homo sapiens (claudin-1 (CLDN1) mRNA, complete cds) (E=2.9e⁻⁹).

A disclosed NOV4a polypeptide (SEQ ID NO:18) encoded by SEQ ID NO:17 is 220 amino acid residues and is presented using the one-letter code in Table 4B. Signal P, Psort and/or Hydropathy results predict that NOV4a has no signal peptide and is likely to be localized in the plasma membrane with a certainty of 0.6400. Alternatively, NOV4a may also localize to the Golgi body with acertainty of 0.4600, the endoplasmic reticulum (membrane) with a certainty of 0.3700, or the enoplasmic reticulum (lumen) with a certainty of 0.1000. The most likely cleavage site for NOV4a is between positions 24 and 25: ILS-II.

TABLE 4B
Encoded NOV4a protein sequence

(SEQ ID NO:18)

MALIFRTANQSVGLLLSLLGWILSIITTYLPHWKNLNLDLNEMENWTMGLWQTCVIQEEVGMQCKDFDSFLA
LPAELRVSRILMFLSNGLGFLGLLVSGFGLDCLRIGESQRDLKRRLLILGGILSWASGITALVPVSWVAHKT
VQEFWDENVPDFVPRWEFGEALFLGWFAGLSLLLGGCLLNCAACSSHAPLALGHYAVAQMQTQCPYLEDGTA
DPQV

The disclosed NOV4a amino acid sequence has 84 of 204 amino acid residues (41%) identical to, and 119 of 204 amino acid residues (58%) similar to, the 219 amino acid residue ptnr:SWISSPROT-ACC:Q9Z262 protein from Mus musculus (Mouse) (Claudin-6) (E=1.1e⁻³²).

NOV4a is predicted to be expressed in at least Brain. This information was derived by determining the tissue sources of the sequences that were included in the invention including but not limited to SeqCalling sources, Public EST sources, Literature sources, and/or RACE sources.

In addition, the sequence is predicted to be expressed in Adrenal Gland/Suprarenal gland, Brain, Bronchus, Brown adipose, Cervix, Colon, Coronary Artery, Epidermis, Gall Bladder, Heart, Hippocampus, Islets of Langerhans, Kidney, Liver, Lung, Lung Pleura, Mammary gland/Breast, Oesophagus, Ovary, Oviduct/Uterine Tube/Fallopian tube, Parotid Salivary glands, Peripheral Blood, Placenta, Prostate, Proximal Convoluted Tubule, Respiratory Bronchiole, Skin, Stomach, Substantia Nigra, Thymus, Thyroid, Trachea, Umbilical Vein, Uterus, and Vulva.

NOV4b

A disclosed NOV4b nucleic acid of 694 nucleotides (also referred to as CG56589-01) encoding a novel Claudin-6-like protein is shown in Table 4C. An open reading frame was identified beginning with an ATG codon at nucleotides 11-13 and ending with a TAA codon at nucleotides 671-673. The start and stop codons are in bold letters and the 5′ and 3′ untranslated regions are underlined in Table 4C. Because the start codon is not a traditional initiation codon, NOV4b could be a partial reading frame. NOV4b could extend further in the 5′ direction.

TABLE 4C
NOV4b nucleotide sequence.

(SEQ ID NO:19)

ACCTGTCGCAATGGCTTTAATCTTTAGAACAGCAATGCAATCTGTTGGACTTTTACTATCTCTCCTGGGATG
GATTTTATCCATTATTACAACTTATTTGCCACACTGGAAGAACCTCAACCTGGACTTAAATGAAATGGAAAA
CTGGACCATGGGACTCTGGCAAACCTGTGTCATCCAAGAGGAAGTGGGGATGCAATGCAAGGACTTTGACTC
CTTCCTGGCTTTGCCTGCTGAACTCAGGGTCTCCAGGATCTTAATGTTTCTGTCAAATGGGCTGGGATTTCT
GGGCCTGCTGGTCTCTGGGTTTGGCCTGGACTGTTTGAGAATTGGAGAGAGTCAGAGAGATCTCAAGAGGCG
ACTGCTCATTCTGGGAGGAATTCTGTCCTGGGCCTCGGGAATCACAGCCCTGGTTCCCGTCTCTTGGGTTGC
CCACAAGACGGTTCAGGAGTTCTGGGATGAGAACGTCCCAGACTTTGTCCCCAGGTGGGAGTTTGGGGAGGC
CCTGTTTCTGGGCTGGTTTGCTGGACTTTCTCTTCTGCTAGGAGGGTGTCTGCTCAACTGCGCAGCCTGCTC
CAGCCACGCTCCCCTAGCTTTGGGCCACTATGCAGTGGCGCAAATGCAAACTCAGTGTCCCTACCTGGAAGA
TGGGACAGCAGATCCTCAAGTGTAAGACTCCGACAAGGCCAGAGAT

In a search of public sequence databases, the NOV4b nucleic acid sequence, located on chromsome 4 has 330 of 556 bases (59%) identical to a gb:GENBANK-ID:AF134160|acc:AF134160.1 mRNA from Homo sapiens (claudin-1 (CLDN1) mRNA, complete cds) (E=2.9e⁻⁰⁹).

The disclosed NOV4b polypeptide (SEQ ID NO:20) encoded by SEQ ID NO:19 has 220 amino acid residues and is presented in Table 4D using the one-letter amino acid code. Signal P, Psort and/or Hydropathy results predict that NOV4b has no signal peptide and is likely to be localized the the plasma membrane with a certainty of 0.6400. Alternatively, NOV4b may also localize to the Golgi body with acertainty of 0.4600, the endoplasmic reticulum (membrane) with a certainty of 0.3700 or in the endoplasmic reticulum (lumen) with a certainty of 0.1000. The most likely cleavage site for a NOV4b peptide is between amino acids 24 and 25, at: ILS-II.

TABLE 4D
Encoded NOV4b protein sequence.

(SEQ ID NO:20)

MALIFRTAMQSVGLLLSLLGWILSIITTYLPHWKNLNLDLNEMENWTMGLWQTCVIQEEVGMQCKDFDSFLA
LPAELRVSRILMFLSNGLGFLGLLVSGFGLDCLRIGESQRDLKRRLLILGGILSWASGITALVPVSWVAHKT
VQEFWDENVPDFVPRWEFGEALFLGWFAGLSLLLGGCLLNCAACSSHAPLALGHYAVAQMQTQCPYLEDGTA
DPQV

A search of sequence databases reveals that the NOV4b amino acid sequence has 84 of 204 amino acid residues (41%) identical to, and 119 of 204 amino acid residues (58%) similar to, the 219 amino acid residue ptnr:SWISSPROT-ACC:Q9Z262 protein from Mus musculus (Mouse) (Claudin-6) (E=1.1e⁻³²).

NOV4b is predicted to be expressed in at least Brain.

In addition, NOV4b is predicted to be expressed in Adrenal Gland/Suprarenal gland, Brain, Bronchus, Brown adipose, Cervix, Colon, Coronary Artery, Epidermis, Gall Bladder, Heart, Hippocampus, Islets of Langerhans, Kidney, Liver, Lung, Lung Pleura, Mammary gland/Breast, Oesophagus, Ovary, Oviduct/Uterine Tube/Fallopian tube, Parotid Salivary glands, Peripheral Blood, Placenta, Prostate, Proximal Convoluted Tubule, Respiratory Bronchiole, Skin, Stomach, Substantia Nigra, Thymus, Thyroid, Trachea, Umbilical Vein, Uterus, and Vulva.

NOV4c

A disclosed NOV4c nucleic acid of 694 nucleotides (also referred to as CG56589-02) encoding a novel Claudin 6-like protein is shown in Table 4E. An open reading frame was identified beginning with an ATG codon at nucleotides 11-13 and ending with a TAA codon at nucleotides 671-673. The start and stop codons are in bold letters and the 5′ and 3′ untranslated regions are underlined in Table 4E.

TABLE 4E
NOV4c nucleotide sequence.

(SEQ ID NO:21)

ACCTGTCGCAATGGCTTTAATCTTTAAAACAGCAATGCAATCTGTTGGACTTTTGCTATCTTTCCTGGGATG
GATTTTATCCATTATTACAACTTATTTGCCACACTGGAAGAACCTCAACCTGGACTTAAATGAAATGGAAAA
CTGGACCATGGGACTCTCGCAAACCTGTGTCATCCAAGAGGAAGTGGGGATGCAATGCAAGGACTTTGACTC
CTTCCTGGCTTTGCCTGCTCAACTCAGGGTCTCCAGGATCTTAATGTTTCTGTCAAATGGGCTGGGATTTCT
GGGCCTGCTGGTCTCTGGGTTTGGCCTGGACTGTTTGAGAATTGGAGAGAGTCAGAGAGATCTCAAGAGGCG
ACTGCTCATTCTGGGAGGAATTCTGTCCTGGGCCTCGGGAATCACGGCCCTGGTTCCCGTCTCTTCGGTTGC
CCACAAGACGGTTCAGGAGTTCTGGGATGAGAACGTCCCAGACTTTGTCCCCAGGTGGGAGTTTGGGGAGGC
CCTGTTTCTGGGCTGGCTTGCTGGACTTTCTCTTCTGCTAGGAGGGTGTCTGCTCAACTGCGCAGCCTGCTC
CAGCCACGCTCCCCTAGCTTTGGGCCACTATGCAGTGGCGCAAATGCAAACTCACTGTCCCTACCTGGAAGA
TGGGACAGCAGATCCTCAAGTGTAAGACTCCGACAAGGCCAGAGAT

In a search of public sequence databases, the NOV4c nucleic acid sequence, located on chromsome 4 has 331 of 556 bases (59%) identical to a gb:GENBANK-ID:AF134160|acc:AF134160.1 mRNA from Homo sapiens (claudin-1 (CLDN1) mRNA, complete cds) (E=3.2e⁻⁹).

The disclosed NOV4c polypeptide (SEQ ID NO:22) encoded by SEQ ID NO:21 has 220 amino acid residues and is presented in Table 4F using the one-letter amino acid code. Signal P, Psort and/or Hydropathy results predict that NOV4c has no signal peptide and is likely to be localized the the plasma membrane with a certainty of 0.6400. Alternatively, NOV4c may also localize to the Golgi body with acertainty of 0.4600, the endoplasmic reticulum (membrane) with a certainty of 0.3700 or in the endoplasmic reticulum (lumen) with a certainty of 0.1000. The most likely cleavage site for a NOV4c peptide is between amino acids 24 and 25, at: ILS-II.

TABLE 4F
Encoded NOV4c protein sequence.

(SEQ ID NO:22)

MALIFKTAMQSVGLLLSFLGWILSIITTYLPHWKNLNLDLNEMENWTMGLWQTCVIQEEVGMQCKDFDSFLA
LPAELRVSRILMFLSNGLGFLGLLVSGFGLDCLRIGESQRDLKRRLLILGGILSWASGITALVPVSWVAHKT
VQEFWDENVPDFVPRWEFGEALFLGWLAGLSLLLGGCLLNCAACSSHAPLALGHYAVAQMQTHCPYLEDGTA
DPQV

A search of sequence databases reveals that the NOV4c amino acid sequence has 83 of 204 amino acid residues (40%) identical to, and 118 of 204 amino acid residues (57%) similar to, the 219 amino acid residue ptnr:SWISSPROT-ACC:Q9Z262 protein from Mus musculus (Mouse) (Claudin-6) (E=9.6e⁻⁶⁶).

The sequence is predicted to be expressed in the following tissues: Adrenal Gland/Suprarenal gland, Brain, Bronchus, Brown adipose, Cervix, Colon, Coronary Artery, Epidermis, Gall Bladder, Heart, Hippocampus, Islets of Langerhans, Kidney, Liver, Lung, Lung Pleura, Mammary gland/Breast, Oesophagus, Ovary, Oviduct/Uterine Tube/Fallopian tube, Parotid Salivary glands, Peripheral Blood, Placenta, Prostate, Proximal Convoluted Tubule, Respiratory Bronchiole, Skin, Stomach, Substantia Nigra, Thymus, Thyroid, Trachea, Umbilical Vein, Uterus, and Vulva.

NOV4 also has homology to the amino acid sequences shown in the BLASTP data listed in Table 4G.

TABLE 4G
BLAST results for NOV4

| Gene Index/ | | Length | Identity | Positives | |
|---|---|---|---|---|---|
| Identifier | Protein/Organism | (aa) | (%) | (%) | Expect |
| gi\|17437504\|ref\|XP_{—} | similar to | 220 | 220/220 | 220/220 | e−105 |
| 068030.1\| | putative (H. | | (100%) | (100%) | |
| (XM_068030) | sapiens) [Homo | | | | |
| | sapiens] | | | | |
| gi\|17437506\|ref\|XP_{—} | similar to | 220 | 192/212 | 198/212 | 9e−96 |
| 068031.1\| | putative (H. | | (90%) | (92%) | |
| (XM_068031) | sapiens) [Homo | | | | |
| | sapiens] | | | | |
| gi\|12843248\|dbj\|BAB25914.1\| | PMP- | 220 | 158/220 | 182/220 | 3e−70 |
| (AK008821) | 22/EMP/MP20/Claudin | | (71%) | (81%) | |
| | family | | | | |
| | containing | | | | |
| | protein˜data | | | | |
| | source: Pfam, | | | | |
| | source | | | | |
| | key: PF00822, | | | | |
| | evidence: ISS˜putative | | | | |
| | [Mus | | | | |
| | musculus] | | | | |
| gi\|17458947\|ref\|XP_{—} | similar to | 229 | 108/188 | 137/188 | 2e−45 |
| 061964.1\| | putative (H. | | (57%) | (72%) | |
| (XM_061964) | sapiens) [Homo | | | | |
| | sapiens] | | | | |
| gi\|7710002\|ref\|NP_057883.1\| | claudin 1 [Mus | 211 | 72/181 | 105/181 | 1e−27 |
| (NM_016674) | musculus] | | (39%) | (57%) | |

The homology of these sequences is shown graphically in the ClustalW analysis shown in Table 4H.

[image]

Table 4I lists the domain description from DOMAIN analysis results against NOV4. This indicates that the NOV4 sequence has properties similar to those of other proteins known to contain this domain.

TABLE 41
Domain Analysis of NOV4

gnh|Pfam|pfam00822, PMP22_Claudin, PMP-22/EMP/MP20/Claudin family
(SEQ ID NO:802)
CD-Length = 162 residues, 67.3% aligned
Score = 35.0 bits (79), Expect = 0.004

| | | | |
|---|---|---|---|
| NOV 4: | 49 | GLWQTCVIQEEVGM-QCKDFDSFLALPAELRVSRILMFLSNCLGFLGLLVSCFGLDCLRI | 107 |
| | | \|\|\|+ \| \| \|\| \| \|+ + \|\| \|\| \|\| + \|+\| \| \| +\| | |
| Sbjct: | 41 | GLWRNCTTQSCTGQISCKVL----ELNDALQAVQALMILSIILGIISLIVFFFQLFTMRK | 96 |
| NOV 4: | 108 | GESQRDLKRRLLILGGILSWASGITALVPVSWVAHKTVQEFWDENVPDFVCPRWEFGEALF | 167 |
| | | \| \| \|\|+ \|\|+ \|\| \| + +\| \| ++ \|\| + | |
| Sbjct: | 97 | GRR---------FKLAGIIPLVSGLCVLVGASIYTSRIATDF--GNPFTPNRKYSFGYSFI | 146 |
| NOV4: | 168 | LGW 170 | |
| | | \|\|\| | |
| Sbjct: | 147 | LGW 149 | |

The claudins are a family of integral membrane proteins that are major components of tight junction (TJ) strands. When claudins are introduced into cells that lack tight junctions, networks of strands and grooves form at cell-cell contact sites that closely resemble native tight junctions. There are at least 17 members of this family in mammals. Claudin family members share ˜38% amino acid identity, and are predicted to have four transmembrane (TM) domains, which is reminiscent of occludin, although they share no sequence similarity with it. Multiple sequence alignment reveals their sequences to be fairly well conserved in the first and fourth putative TM domains, and in the first and second extracellular loops, but they diverge in the second and third TM domains. Although the sequences of their C-terminal cytoplasmic domains vary, the known family members share a common motif of -Y-V. This has been postulated as a possible binding motif for PDZ domains of other tight junction-associated peripheral membrane proteins, such as ZO-1.

The disclosed NOV4 nucleic acid of the invention encoding a Claudin-6-like protein includes the nucleic acid whose sequence is provided in Table 4A or a fragment thereof. The invention also includes a mutant or variant nucleic acid any of whose bases may be changed from the corresponding base shown in Table 4A while still encoding a protein that maintains its Claudin-6-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 41 percent of the bases may be so changed.

The disclosed NOV4 protein of the invention includes the Claudin-6-like protein whose sequence is provided in Table 4B. The invention also includes a mutant or variant protein any of whose residues may be changed from the corresponding residue shown in Table 4B while still encoding a protein that maintains its Claudin-6-like activities and physiological functions, or a functional fragment thereof. In the mutant or variant protein, up to about 61 percent of the residues may be so changed.

The NOV4 nucleic acids and proteins of the invention are useful in potential therapeutic applications implicated in Von Hippel-Lindau (VHL) syndrome, Cirrhosis, Transplantation, Hemophilia, hypercoagulation, Idiopathic thrombocytopenic purpura, autoimmume disease, allergies, immunodeficiencies, transplantation, Graft vesus host, Alzheimer's disease, Stroke, Tuberous sclerosis, hypercalceimia, Parkinson's disease, Huntington's disease, Cerebral palsy, Epilepsy, Lesch-Nyhan syndrome, Multiple sclerosis, Ataxia-telangiectasia, Leukodystrophies, Behavioral disorders, Addiction, Anxiety, Pain, Neuroprotection, Systemic lupus erythematosus, Autoimmune disease, Asthma, Emphysema, Scleroderma, allergy, and Cancer, and/or other pathologies and disorders of the like. The NOV4 nucleic acid, or fragments thereof, may further be useful in diagnostic applications, wherein the presence or amount of the nucleic acid or the protein are to be assessed.

NOV4 nucleic acids and polypeptides are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. For example the disclosed NOV4 protein have multiple hydrophilic regions, each of which can be used as an immunogen. This novel protein also has value in development of powerful assay system for functional analysis of various human disorders, which will help in understanding of pathology of the disease and development of new drug targets for various disorders.

NOV5

NOV5 includes three novel Monocarboxylate transporter (MCT3)-like proteins disclosed below. The disclosed sequences have been named NOV5a, NOV5b, NOV5c, NOV5d, and NOV5e.

NOV5a

A disclosed NOV5a nucleic acid of 1502 nucleotides (also referred to as CG56635-01) encoding a novel Monocarboxylate transporter (MCT3)-like protein is shown in Table 5a. An open reading frame was identified beginning with an ATG initiation codon at nucleotides 24-26 and ending with a TGA codon at nucleotides 1365-1367. The start and stop codons are in bold letters in Table 5A.

TABLE 5A
NOV5a Nucleotide Sequence

(SEQ ID NO:23)

GTTTCCCCACCCCCCAGACGGCGATGACCCCCCAGCCCGCCGGACCCCCGGATGGGGGCTGGGGCTGGGT
GGTGGCCGCCGCAGCCTTCGCGATAAACGGGCTGTCCTACGGGCTGCTGCGCTCGCTGGGCCTTGCCTTC
CCTGACCTTGCCGAGCACTTTGACCGAAGCGCCCAGGACACTGCGTGGATCAGCGCCCTGGCCTTGGCCG
TGCAGCAGGCAGCCAGCCCCGTGGGCAGCGCCCTGAGCACGCGCTGGGGGGCCCGCCCCGTGGTGATGGT
TGGGGGCGTCCTCGCCTCGCTGGGCTTCGTCTTCTCGGCTTTCGCCAGCGATCTGCTCCATCTCTACCTC
GGCCTGGGCCTCCTCGCTGGCTTTGGTTGGGCCCTGGTGTTCGCCCCCGCCCTAGGCACCCTCTCGCGTT
ACTTCTCCCGCCGTCGAGTCTTGGCGGTGGGGCTGGCGCTCACCGGCAACGGGGCCTCCTCGCTGCTCCT
GGCGCCCGCCTTGCAGCTTCTTCTCGATACTTTCGGCTGGCGGGGCGCTCTGCTCCTCCTCGGCGCGATC
ACCCTCCACCTCACCCCCTGTGGCGCCCTGCTGCTACCCCTGGTCCTTCCTGGAGACCCCCCAGCCCCAC
CGCGTAGTCCCCTAGCTGCCCTCGGCCAGAGTCTGTTCACACGCCGCCCCTTCTCAATCTTTGCTCTAGG
CACAGCCCTGGTTCGGGGCGOGTACTTCGTTCCTTACGTGCACTTGGCTCCCCACGCTTTAGACCGGGGC
CTGGGGGGATACGGAGCAGCGCTGGTGGTGGCCGTGGCTGCGATGGGGGATGCGGGCGCCCGGCTGGTCT
GCGGGTGGCTGGCAGACCAAGGCTGGGTGCCCCTCCCGCGGCTCCTGGCCGTATTCGCGCCTCTGACTGG
GCTGGGGCTGTGGGTGGTGGGGCTGGTGCCCGTGGTGGGCGGCGAAGAGAGCTGGGGGGGTCCCCTGCTG
GCCGCGGCTGTGGCCTATGGGCTGAGCGCGGGGAGTTACGCCCCGCTGGTTTTCGGTGTACTCCCCGGGC
TGGTGGGCGTCGGAGGTGTGGTGCAGGCCACAGGGCTGGTGATGATGCTGATGAGCCTCGGGGGGCTCCT
GGGCCCTCCCCTGTCAGGCTTCCTAAGGGATCAGACAGGAGACTTCACCGCCTCTTTCCTCCTGTCTGGT
TCTTTGATCCTCTCCGGCAGCTTCATCTACATAGGGTTGCCCAGGGCGCTGCCCTCCTGTCGTCCAGCCT
CCCCTCCAGCCACGCCTCCCCCAGAGACGGGGCAGCTGCTTCCCGCTCCCCAGGCAGTCTTGCTGTCCCC
AGGAGGCCCTGGCTCCACTCTGGACACCACTTGTTGATTATTTTCTTGTTTGAGCCCCTCCCCCAATAAA
GAATTTTTATCGGGTTTTCCTGAAACCTCCAACTGTTCACCAATCTAGGACCCTGAAAATATTCTACATA
AGACAGCCACAAAGGCTGGTTCAAACGAACAG

The disclosed NOV5a nucleic acid sequence, located on chromosome 17, has 672 of 1110 bases (60%) identical to a gb:GENBANK-ID:AF132610|acc:AF132610.1 mRNA from Homo sapiens (monocarboxylate transporter MCT3 mRNA, complete cds) (E=1.6e⁻²⁹).

A disclosed NOV5a polypeptide (SEQ ID NO:24) encoded by SEQ ID NO:23 is 447 amino acid residues and is presented using the one-letter amino acid code in Table 5B. Signal P, Psort and/or Hydropathy results predict that NOV5a contains no signal peptide and is likely to be localized in the endoplasmic reticulum (membrane) with a certainty of 0.6850. Alternatively, NOV5a is also likely to be localized to the plasma membrane with a certainty of 0.6400, to the Golgi body with a certainty of 0.4600, or to the endoplasmic reticulum (lumen) with a certainty of 0.1000

TABLE 5B
Encoded NOV5a protein sequence.

(SEQ ID NO:24)

MTPQPAGPPDGCWGWVVAAAAFAINGLSYGLLRSLGLAFPDLAEHFDRSAQDTAWISALALAVQQAASPVGSALS
TRWGARPVVMVGGVLASLGFVFSAFASDLLHLYLGLGLLAGFGWALVFAPALGTLSRYFSRRRVLAVGLALTGNG
ASSLLLAPALQLLLDTFGWRGALLLLGAITLHLTPCCALLLPLVLPGDPPAPPRSPLAALGQSLFTRRAFSIFAL
GTALVGGGYFVPYVHLAPHALDRGLGGYGAALVVAVAANGDAGARLVCGWLADQGWVPLPRLLAVFGALTCLGLW
VVGLVPVVGGEESWGGPLLAAAVAYGLSAGSYAPLVFGVLPGLVGVGGVVQATGLVMMLMSLGGLLGPPLSGFLR
DETGDFTASFLLSGSLILSGSFIYIGLPRALPSCGPASPPATPPPETGELLPAPQAVLLSPGGPGSTLDTTC

The disclosed NOV5a amino acid sequence has 96 of 198 amino acid residues (48%) identical to, and 122 of 198 amino acid residues (61%) similar to, the 504 amino acid residue ptnr:SPTREMBL-ACC:O95907 protein from Homo sapiens (Human) (DJ1039K5.2 (Similar To Monocarboxylate Transporter (MCT3))) (E=1.2e⁻⁶⁷).

NOV5a is predicted to be expressed in at least Adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, retina, salivary gland, skeletal muscle, small intestine, spinal cord, spleen, stomach, testis, thyroid, trachea, uterus.

NOV5b

A disclosed NOV5b nucleic acid of 611 nucleotides (also referred to as CG56635-02) encoding a novel Monocarboxylate transporter 3-like protein is shown in Table 5C. An open reading frame was identified beginning with an ATG initiation codon at nucleotides 6-8 and ending with a TGA codon at nucleotides 500-502. The start and stop codons are in bold letters in Table 5B.

TABLE 5C
NOV5b Nucleotide Sequence

(SEQ ID NO:25)

ACGGCGATGACCCCCCAGCCCGCCGGACCCCCGGATGGGGGCTGGGGCTGGGTCGTGGCGGCCGCACCCT
TCGCGATAAACGGGCTGTCCTACGGGCTGCTGCGCTCGCTGGGCCTTGCCTTCCCTGACCTTGCCGAGCA
CTTTGACCGAAGCGCCCAGGACACTGCGTGGATCAGCGCCCTGCCCCTGGCCGTGCAGCAGGCAGCCAGC
CCCGTGGGCAGCGCCCTGAGCACGCGCTGGGGGGCCCGCCCCGTGGTGATGGTTGGGGGCGTCCTCGCCT
CGCTGGGCTTCGTCTTCTCGGCTTTCGCCAGCGATCTGCTGCATCTCTACCTCGGCCTGGGCCTCCTCGC
TGGCTTCCTAAGGGATGAGACAGGAGACTTCACCGCCTCTTTCCTCCTGTCTGGTTCTTTGATCCTCTCC
GGCAGCTTCATCTACATAGGGTTGCCCAGGGCGCTGCCCTCCTGTGGTCCAGCCTCCCCTCCAGCCACGC
CTCCCCCAGAGACGGGGGAGCTGCTTCCCGCTCCCCAGGCAGTCTTGCTGTCCCCAGGAGGCCCTGGCTC
CACTCTGGACACCACTTGTTTGATTATTTTCTTGTTTGAGCCCCTCCCCCAC

The disclosed NOV5b nucleic acid sequence, located on chromosome 17, has 323 of 520 bases (62%) identical to a gb:GENBANK-ID:AF132610|acc:AF132610.1 mRNA from Homo sapiens (monocarboxylate transporter MCT3 mRNA, complete cds) (E=3.2e⁻¹⁸).

A disclosed NOV5b polypeptide (SEQ ID NO:26) encoded by SEQ ID NO:25 is 191 amino acid residues and is presented using the one-letter amino acid code in Table 5D. Signal P, Psort and/or Hydropathy results predict that NOV5b contains no signal peptide and is likely to be localized in the endoplasmic reticulum (membrane) with a certainty of 0.9325. Alternatively, NOV5b is also likely to be localized to the plasma membrane with a certainty of 0.4960, to the microbody (peroxisome) with a certainty of 0.3200, or to the Golgi body with a certainty of 0.1900 The most likely cleavage site for NOV5b is between positions 38 and 39: GLA-FP.

TABLE 5D
Encoded NOV5b protein sequence.

(SEQ ID NO:26)

MTPQPAGPPDGGWGWVVAAAAFAINGLSYGLLRSLGLAFPDLAEHFDRSAQDTAWISALALAVQQAASPVGSALS
TRWGARPVVMVGGVLASLGFVFSAFASDLLHLYLGLGLLAGFLRDETGDFTASFLLSGSLILSGSFIYIGLPRAL
PSCGPASPPATPPPETGELLPAPQAVLLSPGGPGSTLDTTC

The disclosed NOV5b amino acid sequence has 53 of 110 amino acid residues (48%) identical to, and 72 of 110 amino acid residues (65%) similar to, the 504 amino acid residue ptnr:SPTREMBL-ACC:Q9UBE2 protein from Homo sapiens (Human) (Monocarboxylate Transporter MCT3) (E=2.9e⁻²⁸).

NOV5b is predicted to be expressed in at least the following tissues: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea and uterus.

NOV5c

A disclosed NOV5c nucleic acid of 704 nucleotides (also referred to as CG56635-03) encoding a novel Monocarboxylate transporter 3-like protein is shown in Table 5E. An open reading frame was identified beginning with an ATG initiation codon at nucleotides 28-30 and ending with a TGA codon at nucleotides 673-675. The start and stop codons are in bold letters in Table 5E.

TABLE 5E
NOV5c Nucleotide Sequence

(SEQ ID NO:27)

CGAGCAGCCAGAGGCTGGATCTCAGGGATGCCAGCTCCCCAGCGGAAGCACAGGCGTGGAGGCTTCTCTC
ACAGATGTTTCCCCACCCCGCAGACGGCGATGACCCCCCAGCCCGCCGGACCCCCGGATGGGGGCTGGGG
CTGGGTGGTGGCGGCCGCAGCCTTCGCGATAAACGGGCTGTCCTACGGGCTGCTGCGCTCGCTGGGCCTT
CCCTTCCCTGACCTTGCCGAGCACTTTGACCGAAGCGCCCAGGACACTGCGTGGATCAGCGCCCTGGCCC
TGGCCGTGCAGCAGGCAGCCAGCCCCGTGGGCAGCGCCCTGAGCACGCGCTGGGGGGCCCGCCCCCTGGT
GATGGTTGGGGGCGTCCTCGCCTCGCTGGGCTTCGTCTTCTCGGCTTTCGCCAGCGATCTGCTGCATCTC
TACCTCGGCCTGGGCCTCCTCGCTGGCTTCCTAAGGGATGAGACAGGAGACTTCACCGCCTCTTTCCTCC
TGTCTGGTTCTTTGATCCTCTCCGGCACCTTCATCTACATAGGGTTGCCCAGGGCGCTGCCCTCCTGTGG
TCCAGCCTCCCCTCCAGCCACGCCTCCCCCAGAGACGGGGGAGCTGCTTCCCGCTCCCCAGGCAGTCTTG
CTGTCCCCAGGAGGCCCTGGCTCCACTCTGGACACCACTTGTTGATTATTTTCTTGTTTGAGCCCCTCCC
CCAC

The disclosed NOV5c nucleic acid sequence, located on chromosome 17, has 340 of 547 bases (62%) identical to a gb:GENBANK-ID:AF019111|acc:AF019111.2 mRNA from Mus musculus (monocarboxylate transporter 3 (MCT3) mRNA, complete cds) (E=2.4e⁻¹⁵).

A disclosed NOV5c polypeptide (SEQ ID NO:28) encoded by SEQ ID NO:27 is 215 amino acid residues and is presented using the one-letter amino acid code in Table 5F. Signal P, Psort and/or Hydropathy results predict that NOV5c contains no signal peptide and is likely to be localized in the endoplasmic reticulum (membrane) with a certainty of 0.8500. Alternatively, NOV5c is also likely to be localized to the microbody (peroxisome) with a certainty of 0.6400, to the plasma membrane with a certainty of 0.4400, or to the nucleus with a certainty of 0.3000

TABLE 5F
Encoded NOV5c protein sequence.

(SEQ ID NO:28)

MPAPQRKHRRGGFSHRCFPTPQTAITPQPAGPPDGGWGWVVAAAAFAINGLSYGLLRSLGLAFPDLAEHFDRSAQ
DTAWISALALAVQQAASPVGSALSTRWGARPVVMVGCVLASLCFVFSAFASDLLHLYLGLGLLAGFLRDETCDFT
ASFLLSGSLILSGSFIYIGLPRALPSCGPASPPATPPPETGELLPAPQAVLLSPGGPGSTLDTTC

The disclosed NOV5c amino acid sequence has 53 of 110 amino acid residues (48%) identical to, and 72 of 110 amino acid residues (65%) similar to, the 504 amino acid residue ptnr:SPTREMBL-ACC:Q9UBE2 protein from Homo sapiens (Human) (Monocarboxylate Transporter MCT3) (E=2.9e⁻²⁸).

NOV5c is predicted to be expressed in at least the following tissues: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea and uterus.

NOV5d

A disclosed NOV5d nucleic acid of 1513 nucleotides (also referred to as CG56635-04) encoding a novel Monocarboxylate transporter 3-like protein is shown in Table 5G. An open reading frame was identified beginning with an ATG initiation codon at nucleotides 28-30 and ending with a TGA codon at nucleotides 1444-1446. The start and stop codons are in bold letters in Table 5G.

TABLE 5G
NOV5d Nucleotide Sequence

(SEQ ID NO:29)

CCAGCAGCCAGAGGCTGGATCTCAGGGATGCCAGCTCCCCAGCCGAAGCACAGGCGTGGAGGCTTCTCTC
ACAGATGTTTCCCCACCCCGCAGACCGCGATGACCCCCCAGCCCGCCGGACCCCCGGATGGGGGCTGGGG
CTGGGTCGTGGCGGCCGCAGCCTTCGCGATAAACGGGCTGTCCTACGGCCTGCTGCGCTCGCTGGGCCTT
GCCTTCCCTGACCTTGCCGAGCACTTTGACCGAAGCGCCCAGGACACTGCGTGGATCACCGCCCTGGCCC
TGGCCGTGCAGCAGGCAGCCAGTCCCGTGGGCAGCGCCCTGAGCACGCGCTGGGGGGCCCGCCCCGTGGT
GATGGTTGGGGGCGTCCTCGCCTCGCTGGGCTTCGTCTTCTCGGCTTTCGCCAGCGATCTGCTGCATCTC
TACCTCGGCCTGGGCCTCCTCGCTGGTTTTGGTTGGGCCCTGGTGTTCCCCCCCGCCCTAGGCACCCTCT
CGCGTTACTTCTCCCGCCGTCGAGTCTTGGCGGTGGGGCTGGCGCTCACCGGCAACGGGGCCTCCTCGCT
GCTCCTGGCGCCCGCCTTGCAGCTTCTTCTCGATACTTTCGGCTGGCGGGGCGCTCTGCTCCTCCTCGGC
GCGATCACCCTCCACCTCACCCCCTCTGCCGCCCTGCTGCTACCCCTGGTCCTTCCTGGAGACCCCCCAG
CCCCACCGCGTAGTCCCCTAGCTGCCCTCGGCCTGAGTCTGTTCACACGCCGGGCCTTCTCAATCTTTGC
TCTAGGCACAGCCCTGGTTGGGGGCGGGTACTTCGTTCCTTACGTGCACTTGGCTCCCCACGCTTTAGAC
CGGGGCCTGGGGGGATACGGAGCAGCGCTGGTGGTGGCCGTGGCTGCGATGGGGGATGCGGGCGCCCGGC
TGGTCTGCGCGTGGCTGGCAGACCAAGGCTGGGTGCCCCTCCCGCGGCTGCTGGCCGTATTCGGGGCTCT
GACTGCGCTGGGGCTGTGGGTGGTGGGGCTGGTCCCCGTOGTCGGCGGCGAAGAGAGCTGGGGGGGTCCC
CTGCTGGCCGCGGCTGTGGCCTATGGGCTGAGCGCGGGGAGTTACGCCCCGCTGGTTTTCGGTGTACTCC
CCGGGCTGGTGGCCGTCGGAGGTGTGGTGCAGGCCACAGGCCTGGTGATGATGCTGATGAGCCTCGGGGG
GCTCCTGGGCCCTCCCCTGTCAGGTAAGTTCCTAAGGGATGAGACAGGAGACTTCACCCCCTCTTTCCTC
CTGTCTGGTTCTTTGATCCTCTCCGGCAGCTTCATCTACATAGGGTTGCCCACGGCGCTCCCCTCCTGTG
GTCCAGCCTCCCCTCCAGCCACGCCTCCCCCAGAGACGGGGGAGCTGCTTCCCGCTCCCCAGGCAGTCTT
GCTGTCCCCAGGAGGCCCTGGCTCCACTCTGGACACCACTTGTTGATTATTTTCTTGTTTGAGCCCCTCC
CCCAATAAAGAATTTTTATCGGGTTTTCCTGAAACCTCCAACT

The disclosed NOV5d nucleic acid sequence, located on chromosome 17, has 567 of 940 bases (60%) identical to a gb:GENBANK-ID:HSU81800|acc:U81800.1 mRNA from Homo sapiens (monocarboxylate transporter (MCT3) mRNA, complete cds) (E=6.5e⁻³⁰).

A disclosed NOV5d polypeptide (SEQ ID NO:30) encoded by SEQ ID NO:29 is 472 amino acid residues and is presented using the one-letter amino acid code in Table 5H. Signal P, Psort and/or Hydropathy results predict that NOV5d contains no signal peptide and is likely to be localized in the plasma membrane with a certainty of 0.6000. Alternatively, NOV5d is also likely to be localized to the Golgi body with a certainty of 0.4000, to the endoplasmic reticulum (membrane) with a certainty of 0.3000, or to the microbody (peroxisome) with a certainty of 0.3000

TABLE 5H
Encoded NOV5d protein sequence.

(SEQ ID NO:30)

MPAPQRKNRRGGFSHRCFPTPQTANTPQPAGPPDGGWGWVVAAAAFAINGLSYGLLRSLGLAFPDLAEHFDRSAQ
DTAWISALALAVQQAASPVCSALSTRWGARPVVMVGGVLASLGFVFSAFASDLLHLYLGLGLLAGFGWALVFAPA
LGTLSRYFSRRRVLAVGLALTGNGASSLLLAPALQLLLDTFGWRGALLLLGAITLHLTPCGALLLPLVLPGDPPA
PPRSPLAALGLSLFTRRAFSIFALGTALVGGGYFVPYVHLAPHALDRGLGGYGAALVVAVAAMGDAGARLVCGWL
ADQGWVPLPRLLAVFGALTGLGLWVVGLVPVVGGEESWGGPLLAAAVAYGLSAGSYAPLVFGVLPGLVGVGGVVQ
ATGLVMMLMSLGGLLGPPLSGKFLRDETGDFTASFLLSGSLILSGSFIYIGLPRALPSCGPASPPATPPPETGEL
LPAPQAVLLSPGGPGSTLDTTC

The disclosed NOV5d amino acid sequence has 96 of 198 amino acid residues (48%) identical to, and 122 of 198 amino acid residues (61%) similar to, the 504 amino acid residue ptnr:SPTREMBL-ACC:O95907 protein from Homo sapiens (Human) (DJ1039K5.2 (Similar To Monocarboxylate Transporter (MCT3))) (E=7.9e⁻⁶⁸).

NOV5d is predicted to be expressed in at least the following tissues: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea and uterus.

NOV5e

A disclosed NOV5e nucleic acid of 465 nucleotides (also referred to as CG56635-05) encoding a novel Monocarboxylate transporter 3-like protein is shown in Table 5I. An open reading frame was identified beginning with an ATG initiation codon at nucleotides 7-9 and ending with a TGA codon at nucleotides 436-438. The start and stop codons are in bold letters in Table 5I., and the 5′ and 3′ untranslated regions, if any, are underlined.

TABLE 5I
NOV5e Nucleotide Sequence

(SEQ ID NO:31)

ACGGCGATGACCCCCCAGCCCGCCGGACCCCCGGATGGGGGCTGGGGCTGGGTGGTGGCGGCCGCAGCCT
TCGCGATAAACGGGCTGTCCTACGGGCTGCTGCGCTCGCTGGGCCTTGCCTTCCCTGTCCTTGCCGAGCA
CTTTGACCGAAGCGCCCAGGACACTGCGTGGATCAGCGCCCTGGCCCTGGCCGTGCAGCAGCCAGCCAGC
TTCCTAAGGGATGAGACAGGAGACTTCACCGCCTCTTTCCTCCTGTCTGGTTCTTTGATCCTCTCCGGCA
GCTTCATCTACATAGGGTTGCCCAGGGCGCTGCCCTCCTGTGGTCCAGCCTCCCCTCCAGCCACGCCTCC
CCCAGAGACGGGGGAGCTGCTTCCCGCTCCCCAGGCAGTCTTGCTGTCCCCAGGAGGCCCTGGCTCCACT
CTGGACACCACTTGTTGATTATTTTCTTGTTTGAGCCCCTCCCCC

The disclosed NOV5e nucleic acid sequence, located on chromosome 17, has 351 of 434 bases (80%) identical to a gb:GENBANK-ID:AX083362|acc:AX083362.1 mRNA from Homo sapiens (Sequence 54 from Patent WO0112660) (E=1.6e⁻⁵³).

A disclosed NOV5e polypeptide (SEQ ID NO:32) encoded by SEQ ID NO:31 is 143 amino acid residues and is presented using the one-letter amino acid code in Table 5J. Signal P, Psort and/or Hydropathy results predict that NOV5e contains no signal peptide and is likely to be localized extracellularly with a certainty of 0.5040. Alternatively, NOV5e is also likely to be localized to the endoplasmic reticulum (membrane) with a certainty of 0.1000, to the endoplasmic reticulum (lumen) with a certainty of 0.1000, or to the lysosome (lumen) with a certainty of 0.1000. The most likely cleavage site for NOV5e is between positions 43 and 44: VLA-EH.

TABLE 5J
Encoded NOV5e protein sequence.

(SEQ ID NO:32)

MTPQPAGPPDGGWGWVVAAAAFAINGLSYGLLRSLGLAFPVLAEHFDRSAQDTAWISALALAVQQAASFLRDETG
DFTASFLLSGSLILSGSFIYIGLPRALPSCGPASPPATPPPETGELLPAPQAVLLSPGGPGSTLDTTC

The disclosed NOV5e amino acid sequence has 67 of 68 amino acid residues (98%) identical to, and 67 of 68 amino acid residues (98%) similar to, the 375 amino acid residue ptnr:REMTREMBL-ACC:CAC33285 protein from Homo sapiens (Human) (Sequence 54 from Patent WO0112660) (E=2.9e⁻³¹).

NOV5e is predicted to be expressed in at least Mammalian Tissue, Parathyroid Gland, Mammary gland/Breast, Prostate.

NOV5a also has homology to the amino acid sequences shown in the BLASTP data listed in Table 5K.

TABLE 5K
BLAST results for NOV5a

| Gene Index/ | | Length | Identity | Positives | |
|---|---|---|---|---|---|
| Identifier | Protein/Organism | (aa) | (%) | (%) | Expect |
| gi\|7670446\|dbj\|BAA95074.1\| | unnamed protein | 290 | 252/288 | 263/288 | 1e−86 |
| (AB041591) | product [Mus | | (87%) | (90%) | |
| | musculus] | | | | |
| gi\|17491104\|ref\|XP_{—} | similar to solute | 427 | 196/398 | 257/398 | 6e−74 |
| 064368.1\| | carrier family 16 | | (49%) | (64%) | |
| (XM_064368) | (monocarboxylic | | | | |
| | acid | | | | |
| | transporters), | | | | |
| | member 8 (H. | | | | |
| | sapiens) [Homo | | | | |
| | sapiens] | | | | |
| gi\|2497855\|sp\|Q63344\| | MONOCARBOXYLATE | 489 | 142/420 | 220/420 | 6e−53 |
| MOT2_RAT | TRANSPORTER 2 | | (33%) | (51%) | |
| | (MCT 2) | | | | |
| gi\|1432167\|gb\|AAB04023.1\| | monocarboxylate | 489 | 143/420 | 220/420 | 6e−53 |
| (U62316) | transporter 2 | | (34%) | (52%) | |
| | [Rattus | | | | |
| | norvegicus] | | | | |
| gi\|6755536\|ref\|NP_035521.1\| | solute carrier | 484 | 142/421 | 221/421 | 2e−52 |
| (NM_011391) | family 16 | | (33%) | (51%) | |
| | (monocarboxylic | | | | |
| | acid | | | | |
| | transporters), | | | | |
| | member 7 [Mus | | | | |
| | musculus] | | | | |

The homology of these sequences is shown graphically in the ClustalW analysis shown in Table 5J.

[image]

[image]

[image]

[image]

Monocarboxylates such as lactate and pyruvate play a central role in cellular metabolism and metabolic communication between tissues. Essential to these roles is their rapid transport across the plasma membrane, which is catalysed by a recently identified family of proton-linked monocarboxylate transporters (MCTs). Nine MCT-related sequences have so far been identified in mammals, each having a different tissue distribution, whereas six related proteins can be recognized in Caenorhabditis elegans and 4 in Saccharomyces cerevisiae. Direct demonstration of proton-linked lactate and pyruvate transport has been demonstrated for mammalian MCT1-MCT4, but only for MCT1 and MCT2 have detailed analyses of substrate and inhibitor kinetics been described following heterologous expression in Xenopus oocytes. MCT1 is ubiquitously expressed, but is especially prominent in heart and red muscle, where it is up-regulated in response to increased work, suggesting a special role in lactic acid oxidation. By contrast, MCT4 is most evident in white muscle and other cells with a high glycolytic rate, such as tumour cells and white blood cells, suggesting it is expressed where lactic acid efflux predominates. MCT2 has a ten-fold higher affinity for substrates than MCT1 and MCT4 and is found in cells where rapid uptake at low substrate concentrations may be required, including the proximal kidney tubules, neurons and sperm tails. MCT3 is uniquely expressed in the retinal pigment epithelium. MCT1 and MCT4 have been shown to interact specifically with OX-47 (CD147), a member of the immunoglobulin superfamily with a single transmembrane helix. This interaction appears to assist MCT expression at the cell surface

The disclosed NOV5 nucleic acid of the invention encoding a Monocarboxylate transporter (MCT3)-like protein includes the nucleic acid whose sequence is provided in Table 5A, 5C, 5E, 5G, 5I or a fragment thereof. The invention also includes a mutant or variant nucleic acid any of whose bases may be changed from the corresponding base shown in Table 5A, 5C, 5E, 5G, or 5I while still encoding a protein that maintains its Monocarboxylate transporter (MCT3)-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 40 percent of the bases may be so changed.

The disclosed NOV5 protein of the invention includes the Monocarboxylate transporter (MCT3)-like protein whose sequence is provided in Table 5B, 5D, 5F, 5H, or 5J. The invention also includes a mutant or variant protein any of whose residues may be changed from the corresponding residue shown in Table 5B, 5D, 5F, 5H, or 5J while still encoding a protein that maintains its Monocarboxylate transporter (MCT3)-like activities and physiological functions, or a functional fragment thereof. In the mutant or variant protein, up to about 67 percent of the residues may be so changed.

NOV5 nucleic acid and polypeptide show homology to the Monocarboxylate transporter (MCT3) family of proteins. Accordingly, to the NOV5 nucleic acid and polypeptide may function as members of this family. The NOV5 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The nucleic acids and proteins of NOV5 are useful in metabolic disorders such as salla disease, infantile sialic acid storage disease, symptomatic deficiency in lactate transport, subnormal erythrocyte lactate transport, muscle injuries, cystinosis, streptozotocin-induced diabetes, hypoxia, cardiac arrest or stroke, neuronal disorders, retinal angiogenesis, and/or other pathologies and disorders.

NOV5 nucleic acids and polypeptides are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. For example the disclosed NOV5 protein have multiple hydrophilic regions, each of which can be used as an immunogen. This novel protein also has value in development of powerful assay system for functional analysis of various human disorders, which will help in understanding of pathology of the disease and development of new drug targets for various disorders.

NOV6

A disclosed NOV6 nucleic acid of 1336 nucleotides (also referred to CG56674-01) encoding a novel Nitrilase-1-like protein is shown in Table 6A. An open reading frame was identified beginning with an ATG initiation codon at nucleotides 77-79 and ending with a TAA codon at nucleotides 1058-1060. In Table 6A, the 5′ and 3′ untranslated regions are underlined and the start and stop codons are in bold letters.

TABLE 6A
NOV6 Nucleotide Sequence

(SEQ ID NO:33)

GCCCACTCGCTGCGGCCTATCTGGCTCCAGACCGCCCTCCGGATCGGACCCTGCGAATGGTTTTGGCTATA
TCTTCATGCTGGGCTTCATCACCAGGCCTCCTCACAGATTCCTGTCCCTTCTGTGTCCTGGACTCCGGATA
CCTCAACTCTCTGGGGAAGGTGCTCAGCCCAGGCCCAGAGCCATGGCTATCTCCTCTTCCTCCTGCGAAC
GCCCCTGGTGGCTGTGTGCCAGGTAACATCGACGCCAGACAAGCAACAGAACTTTAAAACATGTGCTGAGC
TGGTTCGAGAGGCTGCCAGACTGGGTGCCTGCCTGGCTTTCCTGCCTGAGGCATTTGACTTCATTGCACGG
GACCCTGCAGAGACGCTACACCTGTCTGAACCACTGGGTGGGAAACTTTTGGAAGAATACACCCAGCTTGC
CAGGGAATGTGGACTCTGGCTGTCCTTGGGTGGTTTCCATGAGCGTGGCCAAGACTGGGAGCAGACTCAGA
AAATCTACAATTGTCACGTGCTGCTGAACAGCAAAGGGGCAGTAGTGGCCATTTACAGGAAGACACATCTG
TGTGACGTAGAGATTCCAGGGCAGGGGCCTATGTGTGAAAGCAACTCTACCATGCCTGGGCCCAGTCTTGA
GTCACCTGTCAGCACACCAGCAGGCAAGATTGGTCTAGCTGTCTGCTATGACATGCGGTTCCCTGAACTCT
CTCTGGCATTGGCTCAAGCTGGAACAGAGATACTTACCTATCCTTCAGCTTTTGCATCCATTACAGGCCCA
GCCCACTGGGAGGTGTTGCTGCGGGCCCGTGCTATCGAAACCCAGTGCTATGTAGTGGCAGCAGCACAGTG
TGGACGCCACCATGAGAAGAGAGCAAGTTATGGCCACAGCATGGTGGTAGACCCCTGGGGAACAGTGGTGG
CCCGCTGCTCTGAGGGGCCAGGCCTCTGCCTTGCCCGAATAGACCTCAACTATCTGCGACAGTTGCGCCGA
CACCTGCCTGTGTTCCAGCACCGCAGGCCTGACCTCTATGGCAATCTGGGTCACCCACTGTCTTAAGACTT
GACTTCTGTGACTTTAGACCTGCCCCTCCCACCCCCACCCTGCCACTATGAGCTAGTGCTCATGTGACTTG
GAGGCAGGATCCAGGCACAGCTCCCCTCACTTGGAGAACCTTGACTCTCTTGATGGAACACAGATGGGCTG
CTTGGGAAAGAAACTTTCACCTGAGCTTCACCTGAGGTCAGACTGCAGTTTCAGAAAGGTGGAATTTTATA
TAGTCATTGTTTATTTCATGGAAACTGAAGTTCTGCTGAGGGCTGAGCACCTTCCCCA

The disclosed NOV6 nucleic acid sequence, localized to the p14.2 region of chromosome 3, has 1319 of 1329 bases (99%) identical to a gb:GENBANK-ID:AF069987|acc:AF069987.1 mRNA from Homo sapiens (nitrilase 1 (NIT1) mRNA, complete cds) (E=3.1e⁻²⁹⁰).

A disclosed NOV6 polypeptide (SEQ ID NO:34) encoded by SEQ ID NO:33 is 327 amino acid residues and is presented using the one-letter amino acid code in Table 6B. Signal P, Psort and/or Hydropathy results predict that NOV6 has a signal peptide and is likely to be localized in the cytoplasm with a certainty of 0.4500. Alternatively, NOV6 is also likely to be localized to the microbody (peroxisome) with a certainty of 0.3000, to the lysosome (lumen) with a certainty of 0.2021, or to the mitochondrial matrix space with a certainty of 0.1000. The most likely cleavage site for NOV6 is between positions 27 and 28: LSG-EG

TABLE 6B
Encoded NOV6 protein sequence.

(SEQ ID NO:34)

MLGFITRPPHRFLSLLCPGLRIPQLSGEGAQPRPRAMAISSSSCELPLVAVCQVTSTPDKQQNFKTCAELV
REAARLGACLAFLPEAFDFIARDPAETLHLSEPLGGKLLEEYTQLARECGLWLSLGGFHERGQDWEQTQKI
YNCHVLLNSKGAVVAIYRKTHLCDVEIPGQGPMCESNSTMPGPSLESPVSTPAGKIGLAVCYDMRFPELSL
ALAQAGTEILTYPSAFGSITGPAHWEVLLRARAIETQCYVVAAAQCGRHHEKRASYGHSMVVDPWGTVVAR
CSEGPGLCLARIDLNYLRQLRRHLPVFQHRRPDLYGNLGHPLS

The disclosed NOV6 amino acid sequence has 322 of 327 amino acid residues (98%) identical to, and 322 of 327 amino acid residues (98%) similar to, the 327 amino acid residue ptnr:SPTREMBL-ACC:O76091 protein from Homo sapiens (Human) (Nitrilase Homolog 1) (E=4.5e⁻¹⁷⁶).

NOV6 also has homology to the amino acid sequence shown in the BLASTP data listed in Table 6C.

TABLE 6C
BLAST results for NOV6

| Gene Index/ | | Length | Identity | Positives | |
|---|---|---|---|---|---|
| Identifier | Protein/Organism | (aa) | (%) | (%) | Expect |
| gi\|5031947\|ref\|NP_005591.1\| | nitrilase 1 [Homo | 327 | 322/327 | 322/327 | 0.0 |
| (NM_005600) | sapiens] | | (98%) | (98%) | |
| gi\|3242980\|gb\|AAC40184.1\| | nitrilase homolog | 323 | 272/327 | 298/327 | e−154 |
| (AF069985) | 1 [Mus musculus] | | (83%) | (90%) | |
| gi\|6754856\|ref\|NP_036179.1 | nitrilase 1 [Mus | 323 | 272/327 | 297/327 | e−153 |
| (NM_012049) | musculus] | | (83%) | (90%) | |
| gi\|18204913\|gb\|AAH21634.1\| | Unknown (protein | 323 | 271/327 | 296/327 | e−153 |
| AAH21634 | for MGC: 13825) | | (82%) | (89%) | |
| (BC021634) | [Mus musculus] | | | | |
| gi\|12836591\|dbj\|BAB23723.1\| | data source: MGD, | 290 | 251/288 | 272/288 | e−145 |
| (AK004988) | source | | (87%) | (94%) | |
| | key: MGI: 1350916, | | | | |
| | evidence: ISS˜nitrilase | | | | |
| | 1˜putative | | | | |
| | [Mus musculus] | | | | |

The homology of these sequences is shown graphically in the ClustalW analysis shown in Table 6D.

[image]

[image]

Tables 6E list the domain description from DOMAIN analysis results against NOV6. This indicates that the NOV6 sequence has properties similar to those of other proteins known to contain this domain.

TABLE 6E
Domain Analysis of NOV6

gnl|Pfam|ptam00795, CN_hydrolase, Carbon-nitrogen hydrolase. This
family contains hydrolases that break carbon-nitrogen bonds. The
family includes: Nitrilase EC:3.5.5.1, Aliphatic amidase EC:3.S.1.4,
Biotidinase EC:3.5.1.12, Beta-ureidopropionase EC:3.5.1.6. (SEQ ID NO: 803)
CD-Length = 267 residues, 100.0% aligned
Score = 273 bits (698), Expect = 1e−74

| | | | |
|---|---|---|---|
| NOV 6: | 51 | VCQVTSTP-DKQQNFKTCAELVREAARLGACLAFLPEAFDFI---ARDPAETLHLSEPLQ | 106 |
| | | \| \| \| \| + \|\|+ \|\| \| \|\|\|\| + \|\| +\| + | |
| Sbjct: | 1 | AVQAEPVPEDLAANLQKAEELIEEAAXAGAELVVFPEAFIPGYPYCKSDAEYYENAEAID | 60 |
| NOV 6: | 107 | GKLLEEYTQLARECGLWLSLGGFHERGQDWEQTQKIYNCHVLLNSKGAVVAIYRKTHLCD | 166 |
| | | \|+ + ++\|\|\|+ \|+ + \|\| \|+ \|+\|\| \|\|++ \| ++ \|\|\| \|\| | |
| Sbjct: | 61 | GEETQFLSRLARKNGIVIVLGVSEREGEG-----KLYNTAVLIDPDCKLIOKYRKIHLFT | 115 |
| NOV 6: | 167 | V---EIPGOGPMCESNSTMPGPSLESPVSTPAGKIGLAVCYDMRFPELSLALAOAGTEIL | 223 |
| | | ++ \|+\| \| \| \|\| \|\|+\|\|+\|\| +\|\|\|+\|\|\|+\|\|\|\|\|+ \|\|\| \| \|\|\| | |
| Sbjct: | 116 | DPERKVYGEG----------GGSGFPVFDTPVGKLGLLICYDIRFPELARALALKGAEIL | 165 |
| NOV 6: | 224 | TYPSAFCSITCPAHWEVLLRARAIETQCYVVAAAQCGRHNEKRA-----SYGHSMVVDPW | 278 |
| | | +\|\|\|\|\| \|\| +\|\|\|+\| \|\|\|\|\|\| \|\|+\| \|\| \| \| + \|\|\|\|\|++\|\| | |
| Sbjct: | 166 | AWPSAFGRKTGDSHWELLARARAIENQCFVAAANQVGTEEDLDLFDLGEFYGHSMIIDPD | 225 |
| NOV 6: | 279 | GTVVA-RCSEGPGLCLARIDLNYLRQLRRHLPVFQERRPDLY 319 | |
| | | \| \|+\| \| \|\| +\| \|\|\|+ + + \|+ + \|\|\|\|\|\|\| | |
| Sbjct: | 226 | GKVLAAPAEEEEGLIIADIDLSRIAEARQKMDFLGHRRPDLY 267 | |

The tumor suppressor gene FHIT encompasses the common human chromosomal fragile site at 3p14.2 and numerous cancer cell biallelic deletions. In human and mouse, the nitrilase homologs and Fhit are encoded by two different genes: FHIT and NIT1, localized on chromosomes 3 and 1 in human, and 14 and 1 in mouse, respectively.

Bacterial and plant nitrilases are enzymes that cleave nitrites and organic amides to the corresponding carboxylic acids plus ammonia. The NIT1 gene is expressed as alternatively spliced transcripts. The major NIT1 transcript encodes a deduced 327-amino acid protein that shares 90% amino acid sequence identity with mouse Nit1, 58% identity with the nitrilase domain of C. elegans NitFhit, and 53% identity with the nitrilase domain of Drosophila NitFhit. The NIT1 gene spans approximately 3.2 kb and contains 7 exons. Northern blot analysis detected NIT1 transcripts of approximately 1.4 and 2.4 kb in all adult tissues examined, namely heart, brain, lung, liver, pancreas, kidney, skeletal muscle, and placenta. An approximately 1.2-kb NIT1 transcript was found in skeletal muscle and heart.

The loss of Fhit expression in several common human cancers is well documented.

The disclosed NOV6 nucleic acid of the invention encoding a Nitrilase-1-like protein includes the nucleic acid whose sequence is provided in Table 6A or a fragment thereof. The invention also includes a mutant or variant nucleic acid any of whose bases may be changed from the corresponding base shown in Table 6A while still encoding a protein that maintains its Nitrilase-1-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 1 percent of the bases may be so changed.

The disclosed NOV6 protein of the invention includes the Nitrilase-1-like protin whose sequence is provided in Table 6B. The invention also includes a mutant or variant protein any of whose residues may be changed from the corresponding residue shown in Table 6B while still encoding a protein that maintains its Nitrilase-1-like activities and physiological functions, or a functional fragment thereof. In the mutant or variant protein, up to about 18 percent of the residues may be so changed.

The protein homology information, expression pattern, and map location for the Nitrilase-1-like protein and nucleic acid (NOV6) disclosed herein suggest that NOV6 may have important structural and/or physiological functions characteristic of the Nitrilase-1-like family. Therefore, the NOV6 nucleic acids and proteins of the invention are useful in potential diagnostic and therapeutic applications. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo.

The NOV6 nucleic acids and proteins of the invention are useful in potential diagnostic and therapeutic applications implicated in various diseases and disorders described below and/or other pathologies. For example, the compositions of the present invention will have efficacy for treatment of patients suffering from cancer, muscle conditions, disorders and diseases, longevity, and/or other pathologies/disorders. The NOV6 nucleic acid, or fragments thereof, may further be useful in diagnostic applications, wherein the presence or amount of the nucleic acid or the protein are to be assessed.

NOV6 nucleic acids and polypeptides are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. For example the disclosed NOV6 protein have multiple hydrophilic regions, each of which can be used as an immunogen. This novel protein also has value in development of powerful assay system for functional analysis of various human disorders, which will help in understanding of pathology of the disease and development of new drug targets for various disorders.

NOV7

NOV7 includes three novel cleavage signal-1 protein-like proteins disclosed below. The disclosed sequences have been named NOV7a, NOV7b, NOV7c, and NOV7d.

NOV7a

A disclosed NOV7a nucleic acid of 1822 nucleotides (also referred to as CG56613-01) encoding a novel cleavage signal-1 protein-like protein is shown in Table 7A. An open reading frame was identified beginning with an ATG initiation codon at nucleotides 98-100 and ending with a TAA codon at nucleotides 839-841. A putative untranslated region upstream from the initiation codon is underlined in Table 7A. The start and stop codons are in bold letters.

TABLE 7A
NOV7a nucleotide sequence.

(SEQ ID NO:35)

GGGGCTGACGCAGCATTGCCAATTCTAAATCCATCATTTGACTGAGGAGGAGAGGTTTGAAGTTGATCAGCT
CCAGGGTTTGAGAAATTCAGTCCGAATGGAACTTCAGGACCTGGAACTGCAGCTGGAGGAGCGCCTGCTGGG
CCTGGAGGAGCAGCTTCGTGCTGTGCGCATGCCTTCACCCTTCCGCTCCTCCGCACTCATGGGAATGTGTGG
CAGTAGAAGCACTGATAACTTGTCATGCCCTTCTCCATTGAATGTAATGGAACCAGTCACTGAACTGATGCA
GGAGCAGTCATACCTCAAGTCTGAATTGGGCCTGGGACTTGGAGAAATGCGATTTGAAATTCCTCCTGGACA
AAGCTCAGAATCTGTTTTTTCCAAGCAACGATCAGAATCATCTTCTATATGTTCTGGTCCCTCTCATGCTAA
CAGAAGAACTGCAGTACCTTCTACTGCCTCAGTGGGCAAATCCAAAACCCCATTAGTGGCAAGGAAGAAAGT
GTTCCGAGCATCGGTGGCTCTAACGCCAACAGCTCCTTCTAGAACAGGCTCTGTGCAGACACCTCCAGATTT
GGAAAGTTCTGAGGAAGTTGATGCAGCTGAAGGAGCCCCAGAAGTTGTAGGACCTAAATCTGAAGTGGAAGA
AGGGCATGGAAAACTCCCATCAATGCCAGCTGCTGAGGAAATGCATAAAAATGTGGAGCAAGATGAGTTGCA
GCAAGTCATACCGGAGATTAAAGAGTCTATTGTTGGGGAAATCAGACGGGAAATTGTAAGTCGACTTTTGGC
AGCAGTATCTTCAAGTAAAGCGTCTAATTCTAAGCAAGATTATCATTAAACAGAAATTATAGGTTGGCATGG
ATCCTATTAGCTGTGTAATACTGGAATTATCAATGATATGCACTGGTGGAGGTGTTATTTGTGCTTTACAAG
ATACTTGCTGTTGAGCTGGGCTACTGTATACAGTGTACAATGTGTATTTCTTCAACCATATATTTTAAAAAG
ACGTACATAGAAACTTAGGCACTTTGCTATTTCTTTTCTAAACTATCAAAAACTCTAGCAGTTTGAAAAGCC
TAATATTTATTTGTATGTCAATATTTTTCATTTGATTCCCTATTAGAATTAATTTTAAAACTTGAAGACTTC
CAGACTTATCCAACTTATAAATAACATATTTCTTCAGACTAACATCTTAAAACACTGACCTCTATGAGGTAT
TTACTGTGCAATAACTGATTCATTTTTTTCAGAGCTTGAAGCATCCAATGATTTTTCCCTCCACTGCTGTTA
ATTAATGTCACTTCCAAGAAGAAAAACTGTTCTGTTGTAAAAAATATAATTGCTCTTAATTCTTGGGGAGGT
TACTAATAGCAGTAGGATAGAATTTTATGAGGTTACCTACAACTACTTAATGTACTTACACTGTAAGCCTTG
TTGCTTTACCCAAGACAAATGTAATTTTATCATTGCTTATCTAGTATTTTTCTTTTGGAAATGTGCCTTATG
TTAAACACTATGTACTTTTACTTTTTGCATTGTCCAGACTTCTTTATTAGATGGAGATGTTTCTTTTTCTGT
CTTCTAGACTAAATAGAGTATCATCCAAATAATCGGGCCTATGACTTGAATGAATAGAAATGAATAAGCTGG
TGTTTGTTTTTTCAAAATGGAAGTAATTTAGATTTGTTCTCCTCATACATAAAATGATTTTAGTTCAGTTTT
AACCAGTGAAAACTTTGTTTTTATGAAAAAAAAGGAAAATGGTTTCCCATTTGGTTTTATATGTGTTAAATA
AATGTGTAAAGTAACCACCCCC

The disclosed NOV7a nucleic acid sequence, localized to chromosome 2, has 1822 of 1828 bases (99%) identical to a gb:GENBANK-ID:HUMCS1PA|acc:M61199.1 mRNA from Homo sapiens (Human cleavage signal 1 protein mRNA, complete cds) (E=0.0).

The disclosed NOV7a polypeptide (SEQ ID NO:36) encoded by SEQ ID NO:35 has 247 amino acid residues and is presented in Table 7B using the one-letter amino acid code. Signal P, Psort and/or Hydropathy results predict that NOV7a has a signal peptide and is likely to be localized to the cytoplasm with a certainty of 0.6500. Alternatively, NOV7A may also localize to the mitochondrial matrix space with a certainty of 0.1000, or the lysosome (lumen) with a certainty of 0.1000.

TABLE 7B
Encoded NOV7a protein sequence.

(SEQ ID NO:36)

MELQDLELQLEERLLGLEEQLRAVRMPSPFRSSALMGMCGSRSTDNLSCPSPLNVMEPVTELMQEQSYLKSE
LGLGLGEMGFEIPPGESSESVFSKQRSESSSICSGPSHANRRTGVPSTASVGKSKTPLVARKKVFRASVALT
PTAPSRTGSVQTPPDLESSEEVDAAEGAPEVVGPKSEVEEGHGKLPSMPAAEEMHKNVEQDELQQVIREIKE
SIVGEIRREIVSGLLAAVSSSKASNSKQDYH

A search of sequence databases reveals that the NOV7a amino acid sequence has 247 of 249 amino acid residues (99%) identical to, and 247 of 249 amino acid residues (99%) similar to, the 249 amino acid residue ptnr:SWISSPROT-ACC:P28290 protein from Homo sapiens (Human) (Sperm-Specific Antigen 2 (Cleavage Signal-1 Protein) (CS-1)) (E=6.1e⁻¹²⁴).

NOV7a is predicted to be expressed in at least adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea, uterus, Aorta, Ascending Colon, Bone, Cervix, Cochlea, Colon, Dermis, Gall Bladder, Hypothalamus, Islets of Langerhans, Liver, Lung, Lymphoid tissue, Ovary, Parathyroid Gland, Parotid Salivary glands, Pineal Gland, Retina, Right Cerebellum, Skin, Tonsils, Umbilical Vein, Vein, Whole Organism.

NOV7b

In the present invention, the target sequence identified previously, NOV7a, was subjected to the exon linking process to confirm the sequence. PCR primers were designed by starting at the most upstream sequence available, for the forward primer, and at the most downstream sequence available for the reverse primer. In each case, the sequence was examined, walking inward from the respective termini toward the coding sequence, until a suitable sequence that is either unique or highly selective was encountered, or, in the case of the reverse primer, until the stop codon was reached. Such primers were designed based on in silico predictions for the full length cDNA, part (one or more exons) of the DNA or protein sequence of the target sequence, or by translated homology of the predicted exons to closely related human sequences sequences from other species. These primers were then employed in PCR amplification based on the following pool of human cDNAs: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea, uterus. Usually the resulting amplicons were gel purified, cloned and sequenced to high redundancy. The resulting sequences from all clones were assembled with themselves, with other fragments in CuraGen Corporation's database and with public ESTs. Fragments and ESTs were included as components for an assembly when the extent of their identity with another component of the assembly was at least 95% over 50 bp. In addition, sequence traces were evaluated manually and edited for corrections if appropriate. These procedures provide the sequence reported below, which is designated Accession Number NOV7b (6 aminoacid different from NOV7a) and NOV7c (2 aminoacid different from NOV7a).

A disclosed NOV7b nucleic acid of 806 nucleotides (also referred to as CG56613-02) encoding a novel cleavage signal-1 protein-like protein is shown in Table 7C. An open reading frame was identified beginning with an ATG initiation codon at nucleotides 21-23 and ending with a TAA codon at nucleotides 762-764. A putative untranslated region upstream from the initiation codon is underlined in Table 7C. The start and stop codons are in bold letters, and the 5′ and 3′ untranslated regions, if any, are underlined.

TABLE 7C
NOV7b nucleotide sequence.

(SEQ ID NO:37)

GTTTGAGAAATTCAGTCCGAATGGAACTTCAGGACCTGGAACTGCAGCTGGAGGAGCGCCTGCTGGGCCTGG
AGGAGCAGCTTCGTGCTGTGCGCATGCCTTCACCCTTCCGCTCCTCCGCACTCATGGGAATGTGTGGCAGTA
GAAGCGCTGATAACTTGTCATGCCCTTCTCCATTGAATGTAATGGAACCAGTCACTGAACTGATGCAGGAGC
AGTCATACCTGAAGTCTGAATTCGGCCTGGGACTTGGAGAAATGGGATTTGAAATTCCTCCTGGAGAAAGCT
CAGAATCTGTTTTTTCCCAAGCAACATCAGAATCATCTTCTGTATGTTCTGGTCCCTCTCATGCTAACAGAA
GAACTGGAGTACCTTCTACTGTCTCAGTGGGCAAATCCAAAACCCCATTAGTGGCAAGGAAGAAAGTGTTCC
GAGCATCGGTGGCTCTAACGCCAACAGCTCCTTCTAGAACAGGCTCTGTGCAGACACCTCCAGATTTGGAAA
GTTCTGAGGAAGTTGATGCAGCTGAAGGAGCCCCAGAAGTTGTAGGACCTAAATCTGAAGTGGAAGAACGGC
ATGGAAAACTCCCATCAATGCCAGCTGTTGAGGAAATGCATAAAAATGTGGAGCAAGATGAGTTGCAGCAAG
TCATACGGGAGATTAAAGAGTCTATTGTTGGGGAAATCAGACGGGAAATTGTAAGTGGACTTTTGGCAGCAG
TATCTTCAAGTAAAGCGTCTAATTCTAAGCAAGATTATCATTAAACAGAAATTATACGTTGGCATGGATCCT
ATTAGCTGTGTAAT

The disclosed NOV7b nucleic acid sequence, localized to chromosome 2, has 801 of 812 bases (98%) identical to a gb:GENBANK-ID:HUMCS1PA|acc:M61199.1 mRNA from Homo sapiens (Human cleavage signal 1 protein mRNA, complete cds) (E=7.6e⁻¹⁷¹).

The disclosed NOV7b polypeptide (SEQ ID NO:38) encoded by SEQ ID NO:37 has 247 amino acid residues and is presented in Table 7D using the one-letter amino acid code. Signal P, Psort and/or Hydropathy results predict that NOV7b has no signal peptide and is likely to be localized to the cytoplasm with a certainty of 0.6500. Alternatively, NOV7b may also localize to the mitochondrial matrix space with a certainty of 0.1000, or the lysosome (lumen) with a certainty of 0.1000.

TABLE 7D
Encoded NOV7b protein sequence.

(SEQ ID NO:38)

MELQDLELQLEERLLGLEEQLRAVRMPSPERSSALMGMCGSRSADNLSCPSPLNVMEPVTELMQEQSYLKSE
LGLGLGEMGFEIPPGESSESVFSQATSESSSVCSGPSHANRRTGVPSTVSVGKSKTPLVARKKVFRASVALT
PTAPSRTGSVQTPPDLESSEEVDAAEGAPEVVGPKSEVEEGHGKLPSMPAVEEMHKNVEQDELQQVIREIKE
SIVGEIRREIVSGLLAAVSSSKASNSKQDYH

A search of sequence databases reveals that the NOV7b amino acid sequence has 240 of 249 amino acid residues (96%) identical to, and 242 of 249 amino acid residues (97%) similar to, the 249 amino acid residue ptnr:SWISSNEW-ACC:P28290 protein from Homo sapiens (Human) (Sperm-Specific Antigen 2 (Cleavage Signal-1 Protein) (CS-1)) (E=9.7e⁻¹²¹).

NOV7b is predicted to be expressed in at least adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea, uterus, Aorta, Ascending Colon, Bone, Cervix, Cochlea, Colon, Dermis, Gall Bladder, Hypothalamus, Islets of Langerhans, Liver, Lung, Lymphoid tissue, Ovary, Parathyroid Gland, Parotid Salivary glands, Pineal Gland, Retina, Right Cerebellum, Skin, Tonsils, Umbilical Vein, Vein, Whole Organism.

NOV7c

A disclosed NOV7c nucleic acid of 806 nucleotides (also referred to as CG56613-03) encoding a novel cleavage signal-1 protein-like protein is shown in Table 7E. An open reading frame was identified beginning with an ATG initiation codon at nucleotides 21-23 and ending with a TAA codon at nucleotides 762-764. A putative untranslated region upstream from the initiation codon is underlined in Table 7E. The start and stop codons are in bold letters, and the 5′ and 3′ untranslated regions, if any, are underlined.

TABLE 7E
NOV7c nucleotide sequence.

(SEQ ID NO:39)

GTTTCAGAAATTCACTCCGAATGGAACTTCAGGACCTGGAACTGCAGCTGGAGGAGCGCCTGCTGGGCCTGG
AGGAGCAGCTTCGTGCTGTGCGCATGCCTTCACCCTTCCGCTCCTCCGCACTCATGGGAATGTGTGGCAGTA
GAAGCGCTGATAACTTGTCATCCCCTTCTCCATTGAATGTAATCGAACCAGTCACTGAACTGATGCAGCACC
AGTCATACCTGAAGTCTGAATTGGGCCTGGGACTTGGAGAAATGGGATTTGAAATTCCTCCTGGAGAAAGCT
CAGAATCTGTTTTTTCCCAAGCAACATCAGAATCATCTTCTGTATGTTCTGGTCCCTCTCATGCTAACAGAA
GAGCATCGGTGGCTCTAACGCCAACAGCTCCTTCTAGAACAGGCTCTGTGCAGACACCTCCAGATTTGGAAA
GAGCATCGGTGGCTCTAACGCCAACAGCTCCTTCTAGAACAGGCTCTGTCCAGACACCTCCAGATTTGGAAA
GTTCTGAGGAAGTTGATGCAGCTGAAGGAGCCCCAGAAGTTGTAGGACCTAAATCTGAAGTGGAAGAAGGGC
ATGGAAAACTCCCATCAATGCCAGCTGCTGAGGAAATGCATAAAAATGTGGAGCAAGATGAGTTGCAGCAAG
TCATACGGGAGATTAAAGAGTCTATTGTTGGGGAAATCAGACGGGAAATTGTAAGTGGACTTTTGGCAGCAG
TATCTTCAAGTAAAGCGTCTAATTCTAAGCAAGATTATCATTAAACAGAAATTATAGGTTGGCATGGATCCT
ATTAGCTGTGTAAT

The disclosed NOV7c nucleic acid sequence, localized to chromosome 2, has 803 of 812 bases (98%) identical to a gb:GENBANK-ID:HUMCS1PA|acc:M61199.1 mRNA from Homo sapiens (Human cleavage signal 1 protein mRNA, complete cds) (E=1.2e⁻¹⁷¹).

The disclosed NOV7c polypeptide (SEQ ID NO:40) encoded by SEQ ID NO:39 has 247 amino acid residues and is presented in Table 7F using the one-letter amino acid code. Signal P, Psort and/or Hydropathy results predict that NOV7c has no signal peptide and is likely to be localized to the cytoplasm with a certainty of 0.6500. Alternatively, NOV7f may also localize to the mitochondrial matrix space with a certainty of 0.1000, or the lysosome (lumen) with a certainty of 0.1000.

TABLE 7F
Encoded NOV7c protein sequence.

(SEQ ID NO:40)

MELQDLELQLEERLLGLEEQLRAVRMPSPFRSSALMGMCGSRSADNLSCPSPLNVMEPVTELMQEQSYLSKE
LGLGLGEMGFEIPPGESSESVFSQATSESSSVCSGPSHANRRTGVPSTASVGKSKTPLVARKKVFRASVALT
PTAPSRTGSVQTPPDLESSEEVDAAEGAPEVVGPKSEVEEGHGKLPSMPAAEEMHKNVEQDELQQVIREIKE
SIVGEIRREIVSGLLAAVSSSKASNSKQDYH

A search of sequence databases reveals that the NOV7c amino acid sequence has 242 of 249 amino acid residues (97%) identical to, and 244 of 249 amino acid residues (97%) similar to, the 249 amino acid residue ptnr:SWISSNEW-ACC:P28290 protein from Homo sapiens (Human) (Sperm-Specific Antigen 2 (Cleavage Signal-1 Protein) (CS-1)) (E=1.4e⁻¹²¹).

NOV7c is predicted to be expressed in at least adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea, uterus, Aorta, Ascending Colon, Bone, Cervix, Cochlea, Colon, Dermis, Gall Bladder, Hypothalamus, Islets of Langerhans, Liver, Lung, Lymphoid tissue, Ovary, Parathyroid Gland, Parotid Salivary glands, Pineal Gland, Retina, Right Cerebellum, Skin, Tonsils, Umbilical Vein, Vein, Whole Organism.

NOV7d

A disclosed NOV7d nucleic acid of 705 nucleotides (also referred to as 174307820) encoding a novel cleavage signal-1 protein-like protein is shown in Table 7G. An open reading frame was identified beginning with an AGA initiation codon at nucleotides 1-3 and ending with nucleotides 703-705. The start codon is in bold letters, and the 5′ and 3′ untranslated regions, if any, are underlined. Because the start codon is not a traditional initiation codon, and there is no stop codon, NOV7d could be a partial open reading frame extending further in the 5′ and 3′ directions.

TABLE 7G
NOV7d nucleotide sequence.

(SEQ ID NO:41)

AGATCTCCCACCATGGAACTTCAGGACCTCGAACTGCAGCTGGAGGAGCGCCTGCTGGGCCTGGAGGAGCAG
CTTCGTGCTGTGCGCATGCCTTCACCCTTCCCCTCCTCCGCACTCATGGGAATGTGTGGCAGTAGAAGCGCT
GATAACTTGTCATGCCCTTCTCCATTGAATGTAATGGAACCAGTCACTGAACTGATGCAGGAGCAGTCATAC
CTGAAGTCTGAATTGGGCCTGGGACTTGGAGAAATGGGATTTGAAATTCCTCCTGGAGAAAGCTCAGAATCT
GTTTTTTCCCAAGCAACATCAGAATCATCTTCTGTATGTTCTGGTCCCTCTCATGCTAACAGAAGAACTGGG
GTACCTTCTACTGCCTCAGTGGGCAAATCCAAAACCCCATTAGTGGCAAGGAAGAAAGTGTTCCGAGCATCG
GTCGCTCTAACCCCAACAGCTCCTTCTAGAACAGGCTCTGTGCAGACACCTCCAGATTTGGAAAGTTCTGAG
GAAGTTGATGCAGCTGAAGGAGCCCCAGAAGTTGTAGGACCTAAATCTGAAGTGGAAGAAGGCCATGGAAAA
CTCCCATCAATGCCAGCTGCTGAGGAAATGCATAAAAATGTCGAGCAAGATGAGTTGCAGCAAGTCATACGG
GAGATTAAAGAGTCTATTGTTGGGGAAATCAGACGGGAAATTGTAAGTGGACTCGAG

The disclosed NOV7d polypeptide (SEQ ID NO:42) encoded by SEQ ID NO:41 has 235 amino acid residues and is presented in Table 7H using the one-letter amino acid code.

TABLE 7H
Encoded NOV7d protein sequence.

(SEQ ID NO:42)

RSPTMELQDLELQLEERLLGLEEQLRAVRMPSPFRSSALMGMCGSRSADNLSCPSPLNVMEPVTELMQEQSY
LKSELGLGLGEMGFEIPPGESSESVFSQATSESSSVCSGPSUANRRTGVPSTASVGKSKTPLVARKKVFRAS
VALTPTAPSRTGSVQTPPDLESSEEVDAAEGAPEVVGPKSEVEEGHGKLPSMPAAEEMHKNVEQDELQQVIR
EIKESIVGEIRREIVSGLE

NOV7e

A disclosed NOV7e nucleic acid of 759 nucleotides (also referred to as 174307820) encoding a novel cleavage signal-1 protein-like protein is shown in Table 7I. An open reading frame was identified beginning with an AGA initiation codon at nucleotides 1-3 and ending with nucleotides 757-759. The start codon is in bold letters, and the 5′ and 3′ untranslated regions, if any, are underlined. Because the start codon is not a traditional initiation codon, and there is no stop codon, NOV7e could be a partial open reading frame extending further in the 5′ and 3′ directions.

TABLE 7I
NOV7e nucleotide sequence.

(SEQ ID NO:323)

AGATCTCCCACCATGGAACTTCAGGACCTGGAACTCCAGCTGGAGGAGCGCCTGCTGGGCCTGGAGGAGCAC
CTTCGTGCTGTGCGCATGCCTTCACCCTTCCGCTCCTCCGCACTCATGGGAATGTGTGGCAGTAGAAGCGCT
GATAACTTGTCATGCCCTTCTCCATTGAATGTAATGGAACCAGTCACTGAACTGATGCAGGAGCAGTCATAC
CTGAAGTCTGAATTGGGCCTCGGACTTGGAGAAATGGGATTTGAAATTCCTCCTGGAGAAAGCTCAGAATCT
GTTTTTTCCCAAGCAACATCAGAATCATCTTCTGTATGTTCTGGTCCCTCTCATGCTAACAGAAGAACTGGA
GTACCTTCTACTGCCTCAGTGGGCAAATCCAAAACCCCATTAGTGGCAAGGAAGAAAGTGTTCCGAGCATCG
GTGGCTCTAACGCCAACAGCTCCTTCTAGAACAGGCTCTGTGCAGACACCTCCAGATTTGGAAAGTTCTGAG
GAAGTTGATCCAGCTCAAGGAGCCCCAGAAGTTGTAGGACCTAAATCTGAAGTGGAAGAAGGGCATGGAAAA
CTCCCATCAATGCCAGCTGCTGAGGAAATGCATAAAAATGTGGAGCAAGATGAGTTGCAGCAAGTCATACGG
GAGATTAAAGAGTCTATTGTTGGGGAAATCAGACGGGAAATTGTAAGTGGACTTTTGGCAGCAGTATCTTCA
AGTAAAGCGTCTAATTCTAAGCAAGATTATCATCTCGAG

The disclosed NOV7e polypeptide (SEQ ID NO:324) encoded by SEQ ID NO:323 has 253 amino acid residues and is presented in Table 7J using the one-letter amino acid code.

TABLE 7J
Encoded NOV7e protein sequence.

(SEQ ID NO:324)

RSPTNELQDLELQLEERLLGLEEQLRAVRMPSPFRSSALMGMCGSRSADNLSCPSPLNVMEPVTELMQEQSY
LKSELGLGLGENGFEIPPGESSESVFSQATSESSSVCSGPSHANRRTGVPSTASVGKSKTPLVARKKVFRAS
VALTPTAPSRTGSVQTPPDLESSEEVDAAEGAPEVVGPKSEVEEGHGKLPSMPAAEEMHKNVEQDELQQVIR
EIKESIVGEIRREIVSGLLAAVSSSKASNSKQDYHLE

NOV7a also has homology to the amino acid sequence shown in the BLASTP data listed in Table 7K.

TABLE 7K
BLAST results for NOV7a

| Gene Index/ | | Length | Identity | Positives | |
|---|---|---|---|---|---|
| Identifier | Protein/Organism | (aa) | (%) | (%) | Expect |
| gi\|15620913\|dbj\|BAB67820.1\| | KIAA1927 protein | 772 | 242/247 | 244/247 | e−109 |
| (AB067514) | [Homo sapiens] | | (97%) | (97%) | |
| gi\|16159686\|ref\|XP_057458.1\| | sperm specific | 727 | 242/247 | 244/247 | e−108 |
| (XM_057458) | antigen 2 [Homo | | (97%) | (97%) | |
| | sapiens] | | | | |
| gi\|15277922\|gb\|AAH12947.1\| | Unknown (protein | 267 | 242/247 | 244/247 | e−102 |
| AAH12947 | for MGC: 21202) | | (97%) | (97%) | |
| (BC012947) | [Homo sapiens] | | | | |
| gi\|5803179\|ref\|NP_006742.1\| | sperm specific | 249 | 247/249 | 247/249 | e−102 |
| (NM_006751) | antigen 2; | | (99%) | (99%) | |
| | KIAA1927 protein | | | | |
| | [Homo sapiens] | | | | |
| gi\|18017599\|ref\|NP_542125.1\| | sperm specific | 264 | 197/248 | 212/248 | 9e−81 |
| (NM_080558) | antigen 2 [Mus | | (79%) | (85%) | |
| | musculus] | | | | |

The homology of these sequences is shown graphically in the ClustalW analysis shown in Table 7L.

[image]

[image]

[image]

[image]

The cleavage signal-1 protein (CS-1), a doublet antigen comprised of approximately 14-kDa and 18-kDa proteins has been shown to be present on the surface of sperm of various mammalian species including humans. Polyclonal antibodies to CS-1 inhibit the early cleavage of fertilized eggs without apparently affecting sperm penetration and pronuclear formation. The human CS-1 cDNA has been cloned and expressed in vitro to obtain the recombinant protein (reCS-1) molecule. The CS-1 cDNA clone has been isolated by immunological screening of a human testis lambda gt11 cDNA library with mono-specific polyclonal antibody against CS-1. The cDNA is 1828 bp long; the start codon assigned to the first ATG (bp 98-100) encodes a protein with 249 amino acid residues terminating at TAA (bp 845-847).

XCS-1 is a maternally expressed gene product that is the Xenopus homologue of the human cleavage signal protein (CS-1). XCS-1 may play an important role in regulating mitosis during early embryogenesis in Xenopus laevis. XCS-1 transcripts have been detected in oocytes. During development the XCS-1 protein has been detected on the membrane and in the nucleus of blastomeres. It has also been detected on the mitotic spindle in mitotic cells and on the centrosomes in interphase cells. Overexpression of myc-XCS-1 in Xenopus embryos results in abnormal mitoses with increased numbers of centrosomes, multipolar spindles, and abnormal distribution of chromosomes. Incomplete cytokinesis resulting in multiple nuclei residing in the same cytoplasm with the daughter nuclei in different phases of the cell cycle has been observed. The phenotype depended on the presence of the N terminus of XCS-1 (aa 1-73) and a consensus NIMA kinase phosphorylation site (aa159-167). Mutations in this site affect the ability of the overexpressed XCS-1 protein to produce the phenotype.

The disclosed NOV7 nucleic acid of the invention encoding a Cleavage signal-1 protein-like protein includes the nucleic acid whose sequence is provided in Table 7A, or a fragment thereof. The invention also includes a mutant or variant nucleic acid any of whose bases may be changed from the corresponding base shown in Table 7A while still encoding a protein that maintains its Cleavage signal-1 protein-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 1 percent of the bases may be so changed.

The disclosed NOV7 protein of the invention includes the Cleavage signal-1 protein-like protein whose sequence is provided in Table 7B. The invention also includes a mutant or variant protein any of whose residues may be changed from the corresponding residue shown in Table 2 while still encoding a protein that maintains its Cleavage signal-1 protein-like activities and physiological functions, or a functional fragment thereof. In the mutant or variant protein, up to about 21 percent of the residues may be so changed.

The invention further encompasses antibodies and antibody fragments, such as F_{ab} or (F_{ab})₂, that bind immunospecifically to any of the proteins of the invention.

The above disclosed information suggests that this Cleavage signal-1 protein-like protein (NOV7) is a member of a “Cleavage signal-1 protein family”. Therefore, the NOV7 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV7 nucleic acids and proteins of the invention are useful in potential therapeutic applications implicated in regulation of the cell cycle during early embryogenesis, and therefore may have potential application in the management of embryonic defects. Additionally, this antigen may also be involved in human immunoinfertility and therefore may have application in the treatment of infertility, and/or other diseases or pathologies.

NOV7 nucleic acids and polypeptides are further useful in the generation of antibodies that bind immuno-specifically to the novel NOV7 substances for use in therapeutic or diagnostic methods. These antibodies may be generated according to methods known in the art, using prediction from hydrophobicity charts, as described in the “Anti-NOVX Antibodies” section below. The disclosed NOV7 protein has multiple hydrophilic regions, each of which can be used as an immunogen. These novel proteins can be used in assay systems for functional analysis of various human disorders, which will help in understanding of pathology of the disease and development of new drug targets for various disorders.

NOV8

A disclosed NOV8 nucleic acid of 2838 nucleotides (also referred to as 153472451) encoding a novel Matriptase-like protein is shown in Table 8A. An open reading frame was identified beginning with an TAG initiation codon at nucleotides 8-10 and ending with a TGA codon at nucleotides 2279-2281. The start and stop codons are in bold letters in Table 8A, and the 5′ and 3′ untranslated regions, if any, are underlined.

TABLE 8A
NOV8 nucleotide sequence.

(SEQ ID NO:43)

GGGGACCATGGGGACCGATCGGGCCCGCAAGOGCCCAGGGGGCCCGAAGGACTTCGGCGCGGGACTCAAGTA
CAACTCCCGCCACGAGAAAGTGAATGGCTTGGAGGAAGGCGTGGAGTTCCTGCCAGTCAACAACGTCAAGAA
GGTGGAAAAGCATGGCCCGGGCCGCTGGGTGGTGCTGGCAGCCGTGCTGATCGGCCTCCTCTTGGTGGAGGA
GGCCGAGCGCGTCATGGCCGAGGAGCGCGTAGTCATGCTGCCCCCGCGGGCGCGCTCCCTGAAGTCCTTTGT
GGTCACCTCAGTGGTGGCTTTCCCCACGGACTCCAAAACAGTACAGAGGACCCAGGACAACAGCTGCAGCTT
TGGCCTGCACGCCCGCGGTGTGGAGCTGATGCGCTTCACCACGCCCGGCTTCCCTGACAGCCCCTACCCCGC
TCATGCCCGCTGCCAGTGGCCCCTGCGGGGGGACGCCGACTCAGTGCTGAGCCTCACCTTCCGCAGCTTTGA
CCTTGCGTCCTGCGACGAICGCGGCAGCGACCTGGTGACGGTGTACAACACCCTGAGCCCCATGGAGCCCCA
CGCCCTGGTGCAGTTGTGTGGCACCTACCCTCCCTCCTACAACCTGACCTTCCACTCCTCCCAGAACGTCCT
GCTCATCACACTGATAACCAACACTGAGCGGCGGCATCCCGGCTTTGAGGCCACCTTCTTCCAGCTGCCTAG
GATGAGCAGCTGTGGAGGCCGCTTACGTAAAGCCCAGGGGACATTCAACAGCCCCTACTACCCAGGCCACTA
CCCACCCAACATTGACTGCACATGGAACATTGAGGTGCCCAACAACCAGCATGTGAAGGTGAGCTTCAAATT
CTTCTACCTGCTGGAGCCCGGCGTGCCTGCGGGCACCTGCCCCAAGGACTACGTGCAGATCAATGGGGACAA
ATACTGCGGAGAGAGGTCCCAGTTCGTCGTCACCAGCAACAGCAACAAGATCACAGTTCGCTTCCACTCAGA
TCAGTCCTACACCGACACCOGCTTCTTAGCTGAATACCTCTCCTACGACTCCAGTGACCCATGCCCGGGGCA
GTTCACGTGCCGCACGGGGCGGTGTATCCGGAAGGAGCTGCGCTGTGATGGCTGGCCCGACTGCACCGACCA
CAGCGATGAGCTCAACTGCAGTTGCGACGCCGGCCACCAGTTCACGTGCAAGAACAAGTTCTGCAAGCCCCT
CTTCTGGGTCTGCGACAGTGTGAACGACTGCGGAGACAACAGCGACGAGCAGGGGTGCAGTTGTCCGGCCCA
GACCTTCAGGTGTTCCAATGGGAAGTGCCTCTCGAAAAGCCAGCAGTGCAATGGGAACGACGACTGTGGGGA
CGGGTCCGACGAGGCCTCCTGCCCCAAGGTGAACGTCGTCACTTGTACCAAACACACCTACCGCTGCCTCAA
TGGGCTCTGCTTGAGCAAGCGCAACCCTGAGTGTGACGGGAAGCAGGACTGTAGCGACGGCTCAGATGAGAA
GCACTGCGACTGTGGGCTGCGGTCATTCACGAGACAGGCTCGTGTTGTTGGGGGCACGGATGCGGATGAGGG
CGAGTGGCCCTGGCAGGTAAGCCTGCATGCTCTGGGCCAGGGCCACATCTGCGGTCCTTCCCTCATCTCTCC
CAACTGGCTGGTCTCTGCCGCACACTCCTACATCGATGACAGAGGATTCAGGTACTCAGACCCCACGCAGTG
GACGGCCTTCCTGGGCTTGCACGACCAGAGCCAGCGCAGCGCCCCTCGCGTGCAGGAGCGCAGGCTCAAGCG
CATCATCTCCCACCCCTTCTTCAATGACTTCACCTTCGACTATGACATCGCGCTGCTGGAGCTGGAGAAACC
GGCAGAGTACAGCTCCATGGTGCGGCCCATCTGCCTGCCGGACACCTCCCATGTCTTCCCTGCCGGCAAGGC
CATCTGGGTCACGGGCTGGGGACACACCCAGTATGGAGGCACTGGCGCGCTGATCCTGCAAAAGGGTGAGAT
CCGCGTCATCAACCAGACCACCTGCGAGAACCTCCTGCCGCAGCAGATCACGCCGCGCATGATGTGCGTGGG
CTTCCTCAGCGGCGGCGTGGACTCCTGCCAGGGTGATTCCGGGGGACCCCTGTCCAGCGTGGAGGCGGATGG
GCGGATCTTCCAGGCCGGTGTGGTGAGCTGGGGAGACGGCTGCGCTCAGAGGAACAAGCCAGGCGTGTACAC
AAGGCTCCCTCTGTTTCGGGACTGGATCAAAGAGAACACTGGGGTATAGGGGCCCGGGCCACCCAAATGTGT
ACACCTGCGGGGCCACCCATCCTCCACCCCAGTGTGCACGCCTGCAGGCTGGAGACTGGACCGCTGACTGCA
CCAGCGCCCCCAGAACATACACTGTGAACTCAATCTCCAGGGCTCCAAATCTGCCTAGAAAACCTCTCGCTT
CCTCAGCCTCCAAAGTGGAGCTGGGAGGTAGAAGGGGAGGACACTGGTGGTTCTACTGACCCAACTGGGGGC
AAAGGTTTGAAGACACAGCCTCCCCCGCCAGCCCCAAGCTGGGCCGAGCCGCGTTTGTGTATATCTGCCTCC
CCTGTCTGTAAGGAGCAGCQGGAACGGAGCTTCGGACCCTCCTCAGTGAAGGTGGTGGGGCTGCCGGATCTG
GGCTGTGGGGCCCTTGGGCCACGCTCTTGAGGAAGCCCAGGCTCCGAGGACCCTGGAAAACAGACGGGTCTG
AGACTGAAATTGTTTTACCAGCTCCCAGGGTGGACTTCAGTGTGTGTATTTGTGTAAATGGGTAAAACAATT
TATTTCTTTTTAAAAAAAAAAAAAAAAAAA

The disclosed NOV8 nucleic acid sequence has 2644 of 2678 bases (98%) identical to a gb:GENBANK-ID:AF118224|acc:AF118224.2 mRNA from Homo sapiens (matriptase mRNA, complete cds) (E=0.0).

The disclosed NOV8 polypeptide (SEQ ID NO:44) encoded by SEQ ID NO:43 has 757 amino acid residues is presented in Table 8B using the one-letter amino acid code. Signal P, Psort and/or Hydropathy results predict that NOV8 has a signal peptide and is likely to be localized in the plasma membrane with a certainty of 0.8110. Alternatively, NOV8 is predicted to be localized to the Golgi body with a certainty of 0.3000, to the endoplasmic reticulum (membrane) with a certainty of 0.2000, or to the microbody (peroxisome) with a certainty of 0.1527. The most likely ceavage site for NOV8 is between positions 8 and 9, ARK-GG.

TABLE 8B
Encoded NOV8 protein sequence.

(SEQ ID NO:44)

MGSDRARKGGGGPKDFGAGLKYNSRHEKVNGLEEGVEFLPVNNVKKVEKHGPGRWVVLAAVLIGLLLVEEAE
RVMAEERVVMLPPRARSLKSFVVTSVVAFPTDSKTVQRTQDNSCSFGLHARGVELMRFTTPGFPDSPYPAHA
RCQWALRGDSDSVLSLTFRSFDLASCDERGSDLVTVYNTLSPMEPHALVQLCGTYPPSYNLTFHSSQNVLLI
TLITNTERRHPGFEATFFQLPRMSSCGGRLRKAQGTFNSPYYPHHYPPNIDCTWNIEVPNNQHVKVSFKFFY
LLEPGVPAGTCPKDYVEINGEKYCGERSQFVVTSNSNKITVRFHSDQSYTDTGFLAEYLSYDSSDPCPGQFT
CRTGRCIRKELRCDGWADCTDHSDELNCSCDAGHQFTCKNKFCKPLFWVCDSVNDCGDNSDEQGCSCPAQTF
RCSNGKCLSKSQQCNGKDDCGDGSDEASCPXVNVVTCTKHTYRCLNGLCLSKGNPECDGKEDCSDGSDEKDC
DCGLRSFTRQARVVGGTDADEGEWPWQVSLHALGQGHICGASLISPNWLVSAAHCYIDDRGFRYSDPTQWTA
FLGLHDQSQRSAPGVQERRLKRIISHPFENDFTFDYDIALLELEKPAEYSSMVRPICLPDASHVFPAGKAIW
VTGWGHTQYGGTGALILQKGEIRVINQTTCENLLPQQITPRMMCVGFLSGGVDSCQCDSGGPLSSVEADGRI
FQAGVVSWGDGCAQRNKPGVYTRLPLFRDWIKENTGV

A BLASTX of NOV8 shows that it has 699 of 729 amino acid residues (95%) identical to, and 702 of 729 amino acid residues (96%) similar to, the 855 amino acid residue ptnr:SPTREMBL-ACC:Q9Y5Y6 protein from Homo sapiens (Human) (Matriptase) (E=0.0).

NOV8 is predicted to be expressed in at least the following tissues: Adrenal Gland/Suprarenal gland, Aorta, Ascending Colon, Bone Marrow, Brain, Bronchus, Cartilage, Colon, Duodenum, Gall Bladder, Heart, Islets of Langerhans, Kidney, Kidney Cortex, Lung, Mammary gland/Breast, Ovary, Pancreas, Parathyroid Gland, Parotid Salivary glands, Peripheral Blood, Pituitary Gland, Placenta, Prostate, Small Intestine, Stomach, Thymus, Thyroid, Tonsils, Uterus, Vulva, Whole Organism.

In addition, NOV8 is predicted to be expressed in breast cancer, according to NOV8 nucleic acids, polypeptides, and antibodies. Accordingly to the invention will have diagnostic and therapeutic applications for the detection of breast cancer.

The disclosed NOV8 polypeptide has homology to the amino acid sequences shown in the BLASTP data listed in Table 8C.

TABLE 8C
BLAST results for NOV8

| Gene Index/ | | Length | Identity | Positives | |
|---|---|---|---|---|---|
| Identifier | Protein/Organism | (aa) | (%) | (%) | Expect |
| gi\|10257390\|gb\|AAG15395.1\| | serine protease | 855 | 691/691 | 691/691 | 0.0 |
| AF057145_1 | TADG15 [Homo | | (100%) | (100%) | |
| (AF057145) | sapiens] | | | | |
| gi\|11415040\|ref\|NP_{—} | suppression of | 855 | 690/691 | 690/691 | 0.0 |
| 068813.1\| | tumorigenicity 14 | | (99%) | (99%) | |
| (NM_021978) | (colon carcinoma, | | | | |
| | matriptase, | | | | |
| | epithin); | | | | |
| | suppression of | | | | |
| | tumorigenicity 14 | | | | |
| | (colon | | | | |
| | carcinoma); | | | | |
| | matriptase [Homo | | | | |
| | sapiens] | | | | |
| gi\|12249015\|dbj\|BAB20376.1\| | prostamin [Homo | 855 | 689/691 | 689/691 | 0.0 |
| (AB030036) | sapiens] | | (99%) | (99%) | |
| gi\|7363445\|ref\|NP_035306.2\| | protease, serine, | 855 | 573/691 | 633/691 | 0.0 |
| (NM_011176) | 14 (epithin) [Mus | | (82%) | (90%) | |
| | musculus] | | | | |
| gi\|16758444\|ref\|NP_{—} | suppression of | 855 | 571/691 | 632/691 | 0.0 |
| 446087.1\| | tumorigenicity 14 | | (82%) | (90%) | |
| (NM_053635) | (colon carcinoma, | | | | |
| | matriptase, | | | | |
| | epithin) [Rattus | | | | |
| | norvegicus] | | | | |

The homology between these and other sequences is shown graphically in the ClustalW analysis shown in Table 8D. In the ClustalW alignment of the NOV8 protein, as well as all other ClustalW analyses herein, the black outlined amino acid residues indicate regions of conserved sequence (i.e., regions that may be required to preserve structural or functional properties), whereas non-highlighted amino acid residues are less conserved and can potentially be altered to a much broader extent without altering protein structure or function.

[image]

[image]

[image]

Tables 8E-8R list the domain descriptions from DOMAIN analysis results against NOV8. This indicates that the NOV8 sequence has properties similar to those of other proteins known to contain this domain.

TABLE 8E
Domain Analysis of NOV8

gnl|Smart|smart00020, Tryp_SPc, Trypsin-like serine protease; Many of
these are synthesised as inactive precursor zymogens that are cleaved
during limited proteolysis to generate their active forms. A few,
however, are active as single chain molecules, and others are inactive
due to substitutions of the catalytic triad residues. (SEQ ID NO: 804)
CD-Length = 230 residues, 100.0% aligned
Score = 259 bits (662), Expect = 4e−70

| | | | |
|---|---|---|---|
| NOV 8: | 516 | RVVGGTDADEGEWPWQVSLHALGQGHICGASLISPNWLVSAAHCYIDDRGFRYSDPTQWT | 575 |
| | | \|+\|\|\|++\|+\|+\|\|\|\|\|\| \| \| \|\| \|\|\|\|\| \|+++\|\|\|\| \| \|+ | |
| Sbjct: | 1 | RIVGGSEANIGSFPWQVSLQYRGGRHFCGGSLISPRWVLTAAHC------VYGSAPSSIR | 54 |
| NOV8: | 576 | AFLGLHDQSQRSAPGVQERRLKRIISHPFFNDFTFDYDIALLELEKPAEYSSMVRPICLP | 635 |
| | | \|\| \|\| \| \| \| ++ ++\| \|\| +\| \|+\| \|\|\|\|\|+\| +\| \| \|\|\|\|\|\|\| | |
| Sbjct: | 55 | VRLGSHDLS--SGEETQTVKVSKVIVHPNYNPSTYDNDIALLKLSEPVTLSDTVRPICLP | 112 |
| NOV 8: | 636 | DASHVFPAGKAIWVTGWGHTQY-GGTGALILQKGEIRVINQTTCENLLPQQ--ITPRMMC | 692 |
| | | + + \|\|\| \|+\|\|\| \| \|+ \|\|+ + +++ \|\| \|\| \|+\| | |
| Sbjct: | 113 | SSGYNVPAGTTCTVSGWGRTSESSGSLPDTLQEVNVPIVSNATCRRAYSGGPAITDNMLC | 172 |
| NOV 8: | 693 | VGFLSGGVDSCQGDSGGPLSSVEADGRIFQAGVVSWG-DGCAQRNKPGVYTRLRLFRDWI | 751 |
| | | \| \| \|\| \|+\|\|\|\|\|\|\|\|\| \| \| \| \|+\|\|\|\| \|\|\|+\|\|\|\|\|\|\|\|\|+ + \|\|\| | |
| Sbjct: | 173 | AOGLEGGKDACQGDSGGPL--VCNDPRWVLVGIVSWGSYGCARPNKPGVYTRVSSYLDWI | 230 |

TABLE 8F
Domain Analysis of NOV8

gnl|Pfam|pfam00089, trypsin, Trypsin. Proteins recognized include all
proteins in families S1, S2A, S2B, S2C, and S5 in the classification
of peptidases. Also included are proteins that are clearly members,
but that lack peptidase activity, such as haptoglobin and protein z
(PRTZ*). (SEQ ID NO:805)
CD-Length = 217 residues, 100.0% aligned
Score = 201 bits (510), Expect = 2e−52

| | | | |
|---|---|---|---|
| NOV8: | 517 | VVGGTDADEGEWPWQVSLHALGQGHICCASLISPNWLVSAAHCYIDDRGFRYSDPTQWTA | 576 |
| | | +\|\|\| +\| \| +\|\|\|\|\|\| + \|\| \|\| \|\|\|\| \|\|+++\|\|\|\| \| + | |
| Sbjct: | 1 | IVGGREAQAGSFPWQVSLQ-VSSGHFCGGSLISENNVLTAAHCV--------SGASSVRV | 51 |
| NOV 8: | 577 | FLGLHDQSQRSAPGVQERRLKRIISHPFFNDFTFDYDIALLELEKPAEYSSMVPPICLPD | 636 |
| | | \|\| \|+ \|+ +\|+\|\| \|\| +\| \| \|\|\|\|\|+\|+ \| \|\|\|\|\|\|\| | |
| Sbjct: | 52 | VLGEHNLGTTEG-TEQKFDVKKIIVHPNYNPDT--NDIALLKLKSPVTLGDTVRPICLPS | 108 |
| NOV 8: | 637 | ASHVFPAGKAIWVTGWGHTQYGGTGALILQKGEIRVINQTTCENLLPQQITPRMMCVGFL | 696 |
| | | \|\| \| \| \|+\|\|\| \|+ \|\| + \|\|+ + ++++ \|\| + +\| \|+\| \| \| \| | |
| Sbjct: | 109 | ASSDLPVGTTCSVSGWGRTKNLGT-SDTLQEVVVPIVSRETCRSAYGGTVTDTMICAGAL | 167 |
| NOV8: | 697 | SGGVDSCQGDSGGPLSSVEADGRIEQAGVVSWGDCCAQRNKPGVYTRLPLFRDWI 751 | |
| | | \|\| \|+\|\|\|\|\|\|\|\|\| \|+\|\|\|\| \|\|\| \| \|\|\|\|\|\|+ + \|\|\| | |
| Sbjct: | 168 | -GGKDACQGDSGGPL----VCSDGELVGIVSWGYGCAVGNYPGVYTRVSRYLDWI 217 | |

TABLE 8G
Domain Analysis of NOV8

gnl|Pfam|pfam00431, CUB, COB domain (SEQ ID NO:806)
CD-Length = 110 residues, 100.0% aligned
Score = 99.0 bits (245), Expect = 9e−22

| | | | |
|---|---|---|---|
| NOV 8: | 242 | CGGRLRRAQGTFNSPYYPGHYPPNIDCTWNIEVPNNQHVKVSFKFFYLLEPOVPAGTCPK | 301 |
| | | \|\|\| \| ++ \|+ +\|\| \|\| \|\|\|\| +\| \| \| \| \|+++\|+ \| \| \| | |
| Sbjct: | 1 | CGGVLTESSGSISSPNYPNDYPPNKECVWTIRAPPCYRVELTFQDFDL----EDHTGCRY | 56 |
| NOV 8: | 302 | DYVEI---------NGEKYCGERSQEVVTSNSNKITVRFHSDQSYTDTGFLAEY 346 | |
| | | \|\|\|\|\| \|+\|\| + \|+\|\|++\|++\| \|\| \| + \|\| \| \| | |
| Sbjct: | 57 | DYVEIRDGDGSSSPLLGKFCGSGPPEDIVSSSNRMTIKFVSDASVSKRGFKATY 110 | |

TABLE 8H
Domain Analysis of NOV8

gn1|Pfam|pfam00431, CUB, CUB domain (SEQ ID NO:806)
CD-Length=110 residues, 90.9% aligned
Score=62.4 bits (150), Expect=9e−11

| | | | |
|---|---|---|---|
| NOV8: | 129 | RFTTPGFPDSPYPAHARCQWALRGDADSVLSLTFRSFDLASCDERGSDLVTVYNTLSPME | 188 |
| | | ++\| +\|+ \|\| + \| \| +\| + \|\|\|+ \|\|\| \| \| + + | |
| Sbjct: | 11 | SISSPNYPN-DYPPNKECVWTIRAPPGYRVELTFQDFDLEDHTGCRYDYVEIRDGDGSSS | 69 |
| NOV 8: | 189 | PHALVQLCGTYPPSYNLTFHSSQNVLLITLITNTERRHPGFEATF 233 | |
| | | \| \| + \|\|+ \|\| \|\| \| + \| +++ \|\|+\|\|+ | |
| Sbjct: | 70 | PL-LGKFCGSGPP---EDIVSSSNRMTIKFVSDASVSKRGFKATY 110 | |

TABLE 8I

Domain Analysis of NOV8

gn1|Smart|smart00042, CUB, Domain first found in Clr, Cls, uEGF, and (SEQ ID NO:807)
bone morphogenetic protein.; This domain is found mostly among
developmentally-regulated proteins. Spermadhesins contain only this
domain.
CD-Length=114 residues, 99.1% aligned
Score=97.4 bits (241), Expect=3e−21

| | | | |
|---|---|---|---|
| NOV 8: | 242 | CGGRLRKAQGTFNSPYYPGHYPPNIDCTWNIEVPNNQHVKVSFKFFYLLEPGVPAGTCPK | 301 |
| | | \|\|\| \| + \|\| \|\| \|\| \|\| \|++\| \| \| \| +++ \| \| \| + \| | |
| Sbjct: | 1 | CGGTLTASSGTITSPNYPNSYPNNLNCVWTISAPPGYRIELKFTDFDLE----SSDNCTY | 56 |
| NOV 8: | 302 | DYVEI-NGE--------KYCG-ERSQFVVTSNSNKITVRFHSDQSYTDTGFLAEYLS | 348 |
| | | \|\|\|\|\| +\| ++\|\| \| +++\|+\|\| +\|\| \| \|\| \| \|\| \| \| + | |
| Sbjct: | 57 | DYVEIYDGPSTSSPLLGRFCGSELPPPIISSSSNSMTVTFVSDSSVQKRGFSARYSA | 113 |

TABLE 8J

Domain Analysis of NOV8

gn1|Smart|smart00042, CUB, Domain first found in Clr, Cls, uEGF, and (SEQ ID NO:807)
bone morphogenetic protein.; This domain is found mostly among
developmentally-regulated proteins. Spermadhesins contain only this
domain.
CD-Length=114 residues, 89.5% aligned
Score=58.5 bits (140), Expect=1e−09

| | | | |
|---|---|---|---|
| NOV8: | 129 | RFTTPGFPDSPYPAHARCQWALRGDADSVLSLTFRSFDLASCDERGSDLVTVYNTLSPME | 188 |
| | | \|+\| +\|+\| \|\| + \| \| + + \| \| \|\|\| \| \| \| \| +\|+ \| | |
| Sbjct: | 11 | TITSPNYPNS-YPNNLNCVWTISAPPGYRIELKFTDFDLESSDNCTYDYVEIYDGPSTSS | 69 |
| NOV8: | 189 | PHALVQLCGTYPPSYNLTFHSSQNVLLITLITNTERRHPGFEATFF | 234 |
| | | \| \| + \|\|+ \| \|\| \| + +\| ++++ + \|\| \| + | |
| Sbjct: | 70 | PL-LGRFCGSELP--PPIISSSSNSMTVTFVSDSSVQKRGFSARYS | 112 |

TABLE 8K

Domain Analysis of NOV8
gn1|Smart|smart00192, LDLa, Low-density lipoprotein
receptor domain (SEQ ID NO:808) class A; Cysteine-rich
repeat in the low-density lipoprotein (LDL) receptor
that plays a central role in mammalian cholesterol
metabolism. The N-terminal type A repeats in LDL
receptor bind the lipoproteins. Other homologous
domains occur in related receptors, including the
very low-density lipoprotein receptor and the LDL
receptor-related protein/alpha 2-macroglobulin
receptor, and in proteins which are functionally
unrelated, such as the C9 component of complement.
Mutations in the LDL receptor gene cause familial
hypercholesterolemia.
CD-Length=38 residues, 94.7% aligned
Score=58.5 bits (140), Expect=1e−09

| | | | |
|---|---|---|---|
| NOV8: | 427 | CPAQTFRCSNGKCLSKSQQCNGKDDCGDGSDEASCP | 462 |
| | | \|\| \|+\| \|\|+\|+ \| \|+\| \|\|\|\|\|\|\|\|\| +\|\| | |
| Sbjct: | 2 | CPPGEFQCKNGRCIPLSWVCDGVDDCGDGSDEENCP | 37 |

TABLE 8L

Domain Analysis of NOV8

| | |
|---|---|
| gn1\|Smart\|smart00192, LDLa, Low-density | |
| lipoprotein receptor domain | (SEQ ID NO:808) |
| class A; Cysteine-rich repeat in the low-density | |
| lipoprotein (LDL) receptor that plays a central | |
| role in mammalian cholesterol metabolism. The N- | |
| terminal type A repeats in LDL receptor bind the | |
| lipoproteins. Other homologous domains occur in | |
| related receptors, including the very low-density | |
| lipoprotein receptor and the LDL receptor-related | |
| protein/alpha 2-macroglobulin receptor, and in | |
| proteins which are functionally unrelated, such | |
| as the C9 component of complement. Mutations in | |
| the LDL receptor gene cause familial | |
| hypercholesterolemia. | |
| CD-Length=38 residues, 92.1% aligned | |
| Score=52.0 bits (123), Expect=1e−07 | |

| | | | |
|---|---|---|---|
| NOV8: | 356 | PGQFTCRTGRCIRKELRCDGWADCTDHSDELNCSC | 390 |
| | | \|\|+\| \|+ \|\|\|\| \|\|\| \|\| \| \|\|\| \|\| | |
| Sbjct: | 4 | PGEFQCKNGRCIPLSWVCDGVDDCGDGSDEENCPS | 38 |

TABLE 8M

Domain Analysis of NOV8

gn1|Smart|smart00192, LDLa, Low-density
lipoprotein receptor domain (SEQ ID NO:808) class
A; Cysteine-rich repeat in the low-density
lipoprotein (LDL) receptor that plays a central
role in mammalian cholesterol metabolism. The N-
terminal type A repeats in LDL receptor bind the
lipoproteins. Other homologous domains occur in
related receptors, including the very low-density
lipoprotein receptor and the LDL receptor-related
protein/alpha 2-macroglobulin receptor, and in
proteins which are functionally unrelated, such as
the C9 component of complement. Mutations in the
LDL receptor gene cause familial
hypercholesterolemia.
CD-Length=38 residues, 89.5% aligned
Score=52.0 hits (123), Expect=1e−07

| | | | |
|---|---|---|---|
| NOV8: | 394 | HQFTCKNKFCKPLFWVCDSVNDCGDNSDEQGCSC | 427 |
| | | +\| \|\|\| \| \|\| \|\|\|\| \|+\|\|\|\| \|\|\|+ \| | |
| Sbjct: | 5 | GEFQCKNGRCIPLSWVCDGVDDCGDGSDEENCPS | 38 |

TABLE 8N

Domain Analysis of NOV8

gn1|smart|smart00192, LDLa, Low-density
lipoprotein receptor domain (SEQ ID NO:808) class
A; Cysteine-rich repeat in the low-density
lipoprotein (LDL) receptor that plays a central
role in mammalian cholesterol metabolism. The N-
terminal type A repeats in LDL receptor bind the
lipoproteins. Other homologous domains occur in
related receptors, including the very low-density
lipoprotein receptor and the LDL receptor-related
protein/alpha 2-macroglobulin receptor, and in
proteins which are functionally unrelated, such
as the C9 component of complement. Mutations in
the LDL receptor gene cause familial
hypercholesterolemia.
CD-Length=38 residues, 94.7% aligned
Score=45.1 bits (105), Expect=1e−05

| | | | |
|---|---|---|---|
| NOV8: | 468 | TCTKHTYRCLNGLCLSKGNPECDGKEDCSDGSDEKDC | 504 |
| | | \|\| ++\| \|\| \|+ \|\|\| +\|\| \|\|\|\|\|++\| | |
| Sbjct: | 1 | TCPPGEFQCKNGRCIPLSWV-CDGVDDCGDGSDEENC | 36 |

TABLE 80

Domain Analysis of NOV8

gn1|fam|pfam00057, ldl_recept_a, Low-density
lipoprotein receptor (SEQ ID NO:809) domain class A
CD-Length=39 residues, 92.3% aligned
Score=53.1 bits (126), Expect=5e−08

| | | | |
|---|---|---|---|
| NOV8: | 427 | CPAQTFRCSNGKCLSKSQQCNGKDDCGDGSDEASCP | 462 |
| | | \| \|+\| +\|+\|+ \| \|+\| \|\| \|\|\|\|\| +\| | |
| Sbjct: | 3 | CGPNEFQCGSGECIPMSWVCDGDPDCEDGSDEKNCA | 38 |

TABLE 8P

Domain Analysis of NOV8

gn1|Pfam|pfam00057, ldl_recept_a, Low-
density lipoprotein receptor (SEQ ID NO:809) domain class A
CD-Length=39 residues, 87.2% aligned
Score=47.4 bits (111), Expect=3e−06

| | | | |
|---|---|---|---|
| NOV8: | 356 | PGQFTCRTGRCIRKELRCDGWADCTDHSDELNCS | 389 |
| | | \| +\| \| +\| \|\| \|\|\| \|\| \| \|\|\| \|\|+ | |
| Sbjct: | 5 | PNEFQCGSGECIPMSWVCDGDPDCEDGSDEKNCA | 38 |

TABLE 8Q

Domain Analysis of NOV8

gn1|Pfam|pfam00057, ldl_recept_a, Low-
density lipoprotein receptor (SEQ ID NO: 809) domain class A
CD-Length=39 residues, 84.6% aligned
Score=44.3 bits (103), Expect 3e−05

| | | | |
|---|---|---|---|
| NOV8: | 394 | HQFTCKNKFCKPLFWVCDSVNDCGDNSDEQGCS | 426 |
| | | ++\| \| + \| \|+ \|\|\|\| \|\| \| \|\|\|+ 1+ | |
| Sbjct: | 6 | NEFQCGSGECIPMSNVCDGDPDCEDGSDERNCA | 38 |

TABLE 8R

Domain Analysis of NOV8

gn1|Pfam|pfam00057, ldl_recept_a, Low-
density lipoprotein receptor (SEQ ID NO:809) domain class A
CD-Length=39 residues, 92.3% aligned
Score=42.0 bits (97), Expect=1e−04

| | | | |
|---|---|---|---|
| NOV8: | 468 | TCTKHTYRCLNGLCLSKGNPECDGKEDCSDGSDEKDC | 504 |
| | | \|\| + ++\| +\| \|+ + \|\|\| \|\| \|\|\|\|\|\|+\| | |
| Sbjct: | 2 | TCGPNEFQCGSGECIPM-SWVCDGDPDCEDGSDEKNC | 37 |

The predicted sequence described here belongs to the leucine-rich repeat protein family. It is homologous to insulin like growth factor binding protein (IGFBP) and RP105, a novel B cell surface molecule. It contains five leucine-rich repeat domains. Leucine-rich repeats (LRRs) are relatively short motifs (22-28 residues in length) found in a variety of cytoplasmic, membrane and extracellular proteins (1). A common property of this protein family involves protein-protein interaction. Other functions of LRR-containing proteins include, for example, binding to enzymes and vascular repair (1). LRRs form elongated non-globular structures and are often flanked by cysteine rich domains. The circulating insulin-like growth factors (IGF-I and -II) occur largely as components of a 140 kDa protein complex with IGF binding protein-3 and the acid-labile subunit (ALS). This ternary complex regulates the metabolic effects of the serum IGFs by limiting their access to tissue fluids.

Because of the presence of the Leucine rich repeat domains and the homology to the IGFBP and RP105, we anticipate that the novel sequence described here will have useful properties and functions similar to these genes.

The NOV8 nucleic acid and polypeptide contain structural motifs (i.e. leucine rich repeat domains) that are characteristics of proteins belonging to the leucine-rich repeat protein family. Accordingly, the various NOV8 nucleic acids and polypeptides of the invention are useful, inter alia, as novel members of this protein family.

The disclosed NOV8 nucleic acid of the invention encoding a Insulin like growth factor binding protein-like protein includes the nucleic acid whose sequence is provided in Table 8A, or a fragment thereof. The invention also includes a mutant or variant nucleic acid any of whose bases may be changed from the corresponding base shown in Table 8A while still encoding a protein that maintains its Insulin like growth factor binding protein-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acid, up to about 2 percent of the bases may be so changed.

The disclosed NOV8 protein of the invention includes the Insulin like growth factor binding protein-like protein whose sequence is provided in Table 8B. The invention also includes a mutant or variant protein any of whose residues may be changed from the corresponding residue shown in Table 8B while still encoding a protein that maintains its Insulin like growth factor binding protein-like activities and physiological functions, or a functional fragment thereof. In the mutant or variant protein, up to about 18 percent of the residues may be so changed.

The invention further encompasses antibodies and antibody fragments, such as F_{ab} or (F_{ab})₂, that bind immunospecifically to any of the proteins of the invention.

The above disclosed information suggests that this Insulin like growth factor binding protein-like protein (NOV8) is a member of a “Insulin like growth factor binding protein family”. Therefore, the NOV8 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV8 nucleic acids and proteins of the invention are useful in potential therapeutic applications implicated in diabetes, obesity, Von Hippel-Lindau (VHL) syndrome, Alzheimer's disease, stroke, tuberous sclerosis, hypercalceimia, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, neuroprotection, cirrhosis, transplantation, hemophilia, hypercoagulation, idiopathic thrombocytopenic purpura, autoimmume disease, allergies, immunodeficiencies, graft versus host disease (GVHD), lymphaedema, and other diseases, disorders and conditions of the like.

NOV8 nucleic acids and polypeptides are further useful in the generation of antibodies that bind immuno-specifically to the novel NOV8 substances for use in therapeutic or diagnostic methods. These antibodies may be generated according to methods known in the art, using prediction from hydrophobicity charts, as described in the “Anti-NOVX Antibodies” section below. The disclosed NOV8 protein has multiple hydrophilic regions, each of which can be used as an immunogen. These novel proteins can be used in assay systems for functional analysis of various human disorders, which will help in understanding of pathology of the disease and development of new drug targets for various disorders.

NOV9

NOV9 includes three novel Neuropeptide Y/Peptide YY receptor-like proteins disclosed below. The disclosed sequences have been named NOV9a, and NOV9b.

NOV9a

A disclosed NOV9a nucleic acid of 2276 nucleotides (also referred to as CG56554-01) encoding a novel Neuropeptide Y/Peptide YY receptor-like protein is shown in Table 9A. An open reading frame was identified beginning with an ATG initiation codon at nucleotides 370-372 and ending with a TAA codon at nucleotides 1549-1551. A putative untranslated region upstream from the initiation codon and downstream from the termination codon is underlined in Table 9A. The start and stop codons are in bold letters.

TABLE 9A
NOV9a nucleotide sequence.

(SEQ ID NO:45)

GGCCAGAACGCGGGGAGCCAGAGGCGGCAGGACCCTAGCGTGGCGCTCCAGCACCCCAGACCGTGGCGGCGC
CTCGCCTTAGGGAAGAGCAAGGGAAGAACTTTATTTGAACCGCGAACATTTTTTGGTCACTGAGATCGAGTC
TCCCAGTGCTTTGGCTTCCCGCCTCTTTATCGTGGGTTTGATCCCTGAGCTGCTCTCCTTTCCCGAACCTCC
CGGGGTGCAGCCTAGAGCCCTCCCGCGCGGCTGACTCCAGAGTAGAGGAAGGGAGGCGGCCTCCGGCTGGTC
CCCCGAAGCCCTCGCTGCCCCGCAGATGCGGATGGCCAGCCAGTAGCGGGCGGTGGCCCCGCGTCCCGGGAG
CGCACAGCAATGCAGGCGCTTAACATTACCCCGGAGCAGTTCTCTCGGCTGCTGCGGGACCACAACCTGACG
CGGGAGCAGTTCATCGCTCTGTACCGGCTGCGACCGCTCGTCTACACCCCAGAGCTGCCGGGACGCGCCAAG
CTGGCCCTCGTGCTCACCGGCGTGCTCATCTTCGCCCTGGCGCTCTTTGGCAATGCTCTGGTGTTCTACGTG
GTGACCCGCAGCAAGGCCATGCGCACCGTCACCAACATCTTTATCTGCTCCTTGGCGCTCAGTGACCTGCTC
ATCACCTTCTTCTGCATTCCCGTCACCATGATCCAGAACATTTCCGACAACTGGCTGGAGGGTGCTTTCATT
TGCAAGATGGTGCCATTTGTCCAGTCTACCGCTGTTGTGACAGAAATCCTCACTATGACCTGCATTGCTGTG
CAAACGCACCAGGGACTTGTGCATCCTTTTAAAATGAAGTGGCAATACACCAACCGAAGGCCTTTCACAATG
CTAGGTGTGGTCTGGCTGGTGGCAGTCATCGTAGGATCACCCATGTGGCACGTGCAACAACTTGAGATCAAA
TATGACTTCCTATATGAAAAGGAACACATCTGCTGCTTAGAAGAGTGGACCAGCCCTGTCCACCAGAAGATC
TACACCACCTTCATCCTTGTCATCCTCTTCCTCCTGCCTCTTATGGAGAAGAAACGAGCTGTCATTATGATG
GTGACAGTGGTGGCTCTCTTTGCTGTGTGCTGGGCACCATTCCATGTTGTCCATATGATGATTGAATACAGT
AATTTTCAAAAGGAATATGATGATGTCACAATCAAGATGATTTTTCCTATCGTGCAAATTATTGGATTTTCC
AACTCCATCTGTAATCCCATTGTCTATGCATTTATGAATGAAAACTTCAAAAAAAATGTTTTGTCTGCAGTT
TGTTATTGCATAGTAAATAAAACCTTCTCTCCAGCACAAACGCATGGAAATTCAGGAATTACAATGATGCGG
AAGAAAGCAAAGTTTTCCCTCAGAGAGAATCCAGTGGAGGAAACCAAAGGAGAAGCATTCAGTGATGCCAAC
ATTGAAGTCAAATTGTGTGAACAGACAGAGGAGAAGAAAAAGCTCAAACGACATCTTGCTCTCTTTAGGTCT
GAACTGGCTGAGAATTCTCCTTTAGACAGTAGGCATTAATTATAACAATATCTTCATAATTAATGCCCTTCA
GATTGTAACCCAAAGAGAAAATTATTTTGAGCAAAGGTCAAATACTCTTTTTATTCTTAAGATGATGACAAG
AAGAAAACAAATCATGTTTCCATTAAAAAATGACACGAGGCTAGTCCAAGTGCAGTGATGTTTACAACCAAT
TGATCACAATCATTTAACAGATTTCTGTGTTCCTTCTCATTCCCACTGCTTCACTTGACTAGCCTTAAAAAA
GCAACATGGAAGGCCAGGCACGGTGCCTCATGCCTGTAATCCCAGCACTTTGGGAGGCCTAGACGGGCGGAT
CACGAGGTCAGGAGATCAAAACCATCCTGGCTAACACGGTCAAACCCCATCTCTGCTAAAAATACAAAAATT
AGCCCGGCGTGGTGGCGGGCACCTGTAGTCCCAGCTACTTGGGAGCCTCAGGCGGGAGAATGGTGTGAACCC
GGGAGGCGGAGCTTGCAGTGATCCGAGATCATGCCACTGCACTCCAGCCTGGGCGAAAGAGCGAGACTCCCC
GTCTCAAAAAAAATTTTTTTGAAAAATTCGTAAACCATACTTTTAAGATTATTTCAGTGGATTTTTAAAAAT
CTTGTACAGAAATCAGGGTTCTTAGCTAGCAGTTTTTCTCCCACGCAGTCACTGTAATGTGACTATGTATTG
CTAGATTGAATAAGAAAATAAAATAATATCTTCTTCCTTGAAAA

In a search of public sequence databases, the NOV9a nucleic acid sequence, localized to chromosome 4, has 372 of 434 bases (85%) identical to a gb:GENBANK-ID:HSA400877|acc:AJ400877.1 mRNA from Homo sapiens (ASCL3 gene, CEGP1 gene, C11orf14 gene, C11orf15 gene, C11orf16 gene and C11orf17 gene) (E=2.5e⁻⁶¹).

The disclosed NOV9a polypeptide (SEQ ID NO:46) encoded by SEQ ID NO:45 has 393 amino acid residues and is presented in Table 9B using the one-letter amino acid code. Signal P, Psort and/or Hydropathy results predict that NOV9a has no signal peptide and is likely to be localized to the plasma membrane with a certainty of 0.6000. Alternatively, NOV9a may also localize to the Golgi body with a certainty of 0.4000, the endoplasmic reticulum (membrane) with a certainty of 0.3000, or in the microbody (peroxisome) with a certainty of 0.3000. The most likely cleavage site for NOV9a is between positions 64 and 65: GNA-LV.

TABLE 9B
Encoded NOV9a protein sequence.

(SEQ ID NO:46)

MQALNITPEQFSRLLRDHNLTREQFIALYRLRPLVYTPELPGRAKLALVLTGVLIFALALFGNALVFYVVTR
SKAMRTVTNIFICSLALSDLLITFFCIPVTMIQNISDNWLEGAFICKMVPFVQSTAVVTEILTMTCIAVERH
QGLVHPFKMKWQYTNRRAFTMLGVVWLVAVIVGSPMWHVQQLEIKYDFLYEKEHICCLEEWTSPVHQKIYTT
FILVILFLLPLMEKKRAVIMMVTVVALFAVCWAPFHVVHMMIEYSNFEKEYDDVTIKMIFAIVQIIGFSNSI
CNPIVYAFMNENFKKNVLSAVCYCIVNKTFSPAQRHGNSGITMMRKKAKFSLRENPVEETKGEAFSDGNIEV
KLCEQTEEKKKLKRHLALFRSELAENSPLDSGH

A search of sequence databases reveals that the NOV9a amino acid sequence has 63 of 184 amino acid residues (34%) identical to, and 107 of 184 amino acid residues (58%) similar to, the 377 amino acid residue ptnr:SPTREMBL-ACC:O73733 protein from Brachydanio rerio (Zebrafish) (Zebra danio) (Neuropeptide Y/Peptide YY Receptor YA) (E=0.0).

NOV9a is predicted to be expressed in at least kidney. This information was derived by determining the tissue sources of the sequences that were included in the invention including but not limited to SeqCalling sources, Public EST sources, Literature sources, and/or RACE sources.

In addition, the sequence is predicted to be expressed in lower small intestine, colon, and pancreas, brain, hypothalamus because of SAGE tags identifed for AI308124 and AI307658, ESTs which match to the sequence of the invention: pancreatic cancer, prostate, prostate cancer, brain, glioblastoma, astrocytoma, normal human luminar mammary epithelial cells, breast cancer, ovary, cystadenoma. The SAGE data is reproduced in Example 5. The sequence is also predicted to be expressed in the following tissues because of the expression pattern of related genes in the Neuropeptide Y/Peptide YY/Orexin/Galanin/Cholecystokinin receptor family.

NOV9b

In the present invention, the target sequence identified previously, NOV9a, was subjected to the exon linking process to confirm the sequence. PCR primers were designed by starting at the most upstream sequence available, for the forward primer, and at the most downstream sequence available for the reverse primer. In each case, the sequence was examined, walking inward from the respective termini toward the coding sequence, until a suitable sequence that is either unique or highly selective was encountered, or, in the case of the reverse primer, until the stop codon was reached. Such primers were designed based on in silico predictions for the full length cDNA, part (one or more exons) of the DNA or protein sequence of the target sequence, or by translated homology of the predicted exons to closely related human sequences sequences from other species. These primers were then employed in PCR amplification based on the following pool of human cDNAs: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea, uterus. Usually the resulting amplicons were gel purified, cloned and sequenced to high redundancy. The resulting sequences from all clones were assembled with themselves, with other fragments in CuraGen Corporation's database and with public ESTs. Fragments and ESTs were included as components for an assembly when the extent of their identity with another component of the assembly was at least 95% over 50 bp. In addition, sequence traces were evaluated manually and edited for corrections if appropriate. These procedures provide the sequence reported below, which is designated NOV9b. This differs from the previously identified sequence (NOV9a) in having 38 less amino acids and 3 different ones.

A disclosed NOV9b nucleic acid of 1472 nucleotides (also referred to as CG56554-02) encoding a novel Neuropeptide Y/Peptide YY receptor-like protein is shown in Table 9C. An open reading frame was identified beginning with an ATG initiation codon at nucleotides 42-44 and ending with a TAA codon at nucleotides 1335-1337. A putative untranslated region upstream from the initiation codon and downstream from the termination codon is underlined in Table 9C. The start and stop codons are in bold letters.

TABLE 9C
NOV9b nucleotide sequence.

(SEQ ID NO:47)
CAGTAGCGGGCGGTGGCCCCGCGTCCCGGGAGCGCACAGCAATGCACGCGCTTAACATTACCCCGGAGCAGT
TCTCTCGGCTGCTGCGGGACCACAACCTGACGCGGGAGCAGTTCATCGCTCTGTACCGGCTGCGACCGCTCG
TCTACACCCCAGAGCTGCCGGGACGCGCCAAGCTGGCCCTCGTGCTCACCGGCGTGCTCATCTTCGCCCTGG
CGCTCTTTGGCAATGCTCTGGTGTTCTACGTGGTGACCCGCAGCAAGGCCATGCGCACCGTCACCAACATCT
TTATCTGCTCCTTCGCGCTCAGTGACCTGCTCATCACCTTCTTCTGCATTCCCGTCACCATGCTCCAGAACA
TTTCCGACAACTGGCTGGGGGGTGCTTTCATTTGCAAGATGGTGCCATTTGTCCAGTCTACCGCTGTTGTGA
CAGAAATCCTCACTATCACCTGCATTGCTGTGGAAAGGCACCAGGGACTTGTGCATCCTTTTAAAATGAAGT
GGCAATACACCAACCGAAGGGCTTTCACAATGCTAGGTGTGGTCTGGCTGGTGGCAGTCATCGTAGGATCAC
CCATGTGGCACGTGCAACAACTTGAGATCAAATATGACTTCCTATATGAAAAGGAACACATCTGCTGCTTAG
AAGAGTGCACCAGCCCTGTGCACCAGAAGATCTACACCACCTTCATCCTTGTCATCCTCTTCCTCCTGCCTC
TTATGGTGATGCTTATTCTGTACAGTAAAATTGGTTATGAACTTTCGATAAAGAAAAGAGTTGGGGATGGTT
CAGTGCTTCGAACTATTCATGGAAAAGAAATGTCCAAAATAGCCAGGAAGAAGAAACGAGCTGTCATTATGA
TGGTGACAGTGGTGGCTCTCTTTGCTGTGTGCTGGGCACCATTCCATGTTGTCCATATGATGATTGAATACA
GTAATTTTGAAAAGCAATATGATGATGTCACAATCAAGATGATTTTTGCTATCGTGCAAATTATTCGATTTT
CCAACTCCATCTGTAATCCCATTGTCTATGCATTTATGAATGAAAACTTCAAAAAAAATGTTTTGTCTGCAG
TTTGTTATTGCATAGTAAATAAAACCTTCTCTCCAGCACAAAGGCATGGAAATTCAGGAATTACAATGATGC
GGAAGAAAGCAAAGTTTTCCCTCAGAGAGAATCCAGTGGAGGAAACCAAAGGAGAAGCATTCAGTGATGGCA
ACATTGAAGTCAAATTGTGTGAACAGACAGAGGAGAAGAAAAAGCTCAAACGACATCTTGCTCTCTTTAGGT
CTGAACTGGCTGAGAATTCTCCTTTAGACAGTGGGCATTAATTATAACAATATCTTCATAATTAATGCCCTT
CAGATTGTAACCCAAAGAGAAAATTATTTTGAGCAAAGGTCAAATACTCTTTTATTCTTAAGATGATGACA
AGAAGAAAACAAATATGTTTCATTAAAAATGA

In a search of public sequence databases, the NOV9b nucleic acid sequence, localized to chromosome 4, has 403 of 656 bases (61%) identical to a gb:GENBANK-ID:AB040103|acc:AB040103.1 mRNA from Rattus norvegicus (Rattus norvegicus OT7T022 mRNA for RFamide-related peptide receptor, complete cds) (E=7.8e⁻¹³).

The disclosed NOV9b polypeptide (SEQ ID NO:48) encoded by SEQ ID NO:47 has 393 amino acid residues and is presented in Table 9D using the one-letter amino acid code. Signal P, Psort and/or Hydropathy results predict that NOV9b has no signal peptide and is likely to be localized to the plasma membrane with a certainty of 0.6000. Alternatively, NOV9b may also localize to the Golgi body with a certainty of 0.4000, the endoplasmic reticulum (membrane) with a certainty of 0.3000, or in the microbody (peroxisome) with a certainty of 0.3000. The most likely cleavage site for NOV9b is between positions 64 and 65: GNA-LV.

TABLE 9D

Encoded NOV9b protein sequence.

(SEQ ID NO:48)

MQALNITPEQFSRLLRDHNLTREQFIALYRLRPLVYTPELPGRAKLALVLTGVLIFALALFGNALVFYVVTR
SKAMRTVTNIFICSLALSDLLITFFCIPVTMIQNISDNWLEGAFICKMVPFVQSTAVVTEILTMTCIAVERH
QGLVHPFKMKWQYTNRRAFTMLGVVWLVAVIVGSPMWHVQQLEIKYDFLYEKEHICCLEEWTSPVHQKIYTT
FILVILFLLPLMEKKRAVIMMVTVVALFAVCWAPFHVVHMMIEYSNFEKEYDDVTIKMIFAIVQIIGFSNSI
CNPIVYAFMNENFKKNVLSAVCYCIVNKTFSPAQRHGNSGITMMRKKAKFSLRENPVEETKGEAFSDGNIEV
KLCEQTEEKKKLKRHLALFRSELAENSPLDSGH

A search of sequence databases reveals that the NOV9b amino acid sequence has 108 of 315 amino acid residues (34%) identical to, and 180 of 315 amino acid residues (57%) similar to, the 522 amino acid residue ptnr:SWISSNEW-ACC:Q9Y5X5 protein from Homo sapiens (Human) (Neuropeptide Ff Receptor 2 (Neuropeptide G Protein-Coupled Receptor) (G-Protein-Coupled Receptor HLWAR77)) (E=5.2e⁻⁴⁶).

NOV9b is predicted to be expressed in at least the following tissues: lower small intestine, colon, and pancreas, brain, hypothalamus, kidney, pancreatic cancer, prostate, prostate cancer, glioblastoma, astrocytoma, normal human luminar mammary epithelial cells, breast cancer, ovary, cystadenoma.

The disclosed NOV9a polypeptide has homology to the amino acid sequences shown in the BLASTP data listed in Table 9E.

TABLE 9E
BLAST results for NOV9a

| Gene Index/ | | Length | Identity | Positives | |
|---|---|---|---|---|---|
| Identifier | Protein/Organism | (aa) | (%) | (%) | Expect |
| gi\|16566347\|gb\|AAL26488.1\| | G protein-coupled | 455 | 382/393 | 384/393 | 0.0 |
| AF411117_1 | receptor [Homo | | (97%) | (97%) | |
| (AF411117) | sapiens] | | | | |
| gi\|13027438\|ref\|NP_{—} | neuropeptide FF | 417 | 99/314 | 157/314 | 3e−37 |
| 076470.1\| | receptor 2 | | (31%) | (49%) | |
| (NM_023980) | [Rattus | | | | |
| | norvegicus] | | | | |
| gi\|4106397\|gb\|AAD02833.1\| | neuropeptide | 374 | 90/320 | 169/320 | 4e−37 |
| (AF073925) | Y/peptide YY | | (28%) | (52%) | |
| | receptor Yb | | | | |
| | [Gadus morhua] | | | | |
| gi\|4758820\|ref\|NP_004876.1\| | neuropeptide G | 522 | 98/317 | 159/317 | 4e−37 |
| (NM_004885) | protein-coupled | | (30%) | (49%) | |
| | receptor; | | | | |
| | neuropeptide FF 2 | | | | |
| | [Homo sapiens] | | | | |
| gi\|13878604\|sp\|Q9Y5X5\| | NEUROPEPTIDE FF | 522 | 98/317 | 159/317 | 4e−37 |
| NFF2_HUMAN | RECEPTOR 2 | | (30%) | (49%) | |
| | (NEUROPEPTIDE G | | | | |
| | PROTEIN-COUPLED | | | | |
| | RECEPTOR) (G- | | | | |
| | PROTEIN-COUPLED | | | | |
| | RECEPTOR HLWAR77) | | | | |

The homology between these and other sequences is shown graphically in the ClustalW analysis shown in Table 9F. In the ClustalW alignment of the NOV9 protein, as well as all other ClustalW analyses herein, the black outlined amino acid residues indicate regions of conserved sequence (i.e., regions that may be required to preserve structural or functional properties), whereas non-highlighted amino acid residues are less conserved and can potentially be altered to a much broader extent without altering protein structure or function.

[image]

[image]

[image]

Tables 9G-9H list the domain descriptions from DOMAIN analysis results against NOV9. This indicates that the NOV9 sequence has properties similar to those of other proteins known to contain this domain.

TABLE 9G
Domain Analysis of NOV9

gn1|Pfam|pfam00001, 7tm_1, 7 transmembrane receptor (rhodopsin
family). (SEQ ID NO:810)
CD-Length = 254 residues, 100.0% aligned
Score = 146 bits (368), Expect = 2e−36

| | | | |
|---|---|---|---|
| NOV 9: | 62 | GNALVFYVVTRSKAMRTVTNIFICSLALSDLLITFFCIPVTMIQNISDNWLEGAFICKMV | 121 |
| | | \|\| \|\| \|+ \|+\| +\|\| \|\|\|\|+ +\|\|++\|\|\| \| + + +\|+ \| +\|\|+\| | |
| Sbjct: | 1 | GNLLVILVILRTKKLRTPTNIFLLNLAVADLLFLLTLPPWALYYLVGGDWVFGDALCKLV | 60 |
| NOV 9: | 122 | PFVQSTAVVTEILTMTCIAVERHQGLVHPFKMKWQYTNRRAFTMLGVVWLVAVIVGSPMW | 181 |
| | | + \|\| +\| \|++++\|+ +\|\|\| + + \| \|\|\| ++ +\|\|++\|+++ \| | |
| Sbjct: | 61 | GALFVVNGYASILLLTAISIDRYLAIVHPLRYRRIRTPRRAKVLILLVWVLALLLSLPPL | 120 |
| NOV 9: | 182 | HVQQLEIKYDFLYEKEHICCLEEWTSPVHQKIYTTFILVILFLLPL-------------- | 227 |
| | | \| + \| \|\| ++ ++ \| ++ \|+\|\|\| | |
| Sbjct: | 121 | LFSWLR----TVEEGNTTVCLIDFPEESVKRSYVLLSTLVGFVLPLLVILVCYTRILRTL | 176 |
| NOV 9: | 228 | MEKKRAVIMMVTVVALFAVCWAPFHVVHMMIEYSNFEKEYDDVTIK | 273 |
| | | +++\| \|++ \|\| \|+\|+\| ++ + + | |
| Sbjct: | 177 | RKRARSQRSLKRRSSSERKAAKMLLVVVVVFVLCWLPYHIVLLL---DSLCLLSIWRVLP | 233 |
| NOV9: | 274 | MIFAIVQIIGFSNSICNPIVY 294 | |
| | | \| + + \|\| \|\|\|+\| | |
| Sbjct: | 234 | TALLITLWLAYVNSCLNPIIY 254 | |

TABLE 9H
Domain Analysis of NOV9

gn1|Pfam|pfam01604, 7tm_5, 7TM chemoreceptor. This large family of
proteins are related to pfam00001. They are 7 transmembrane receptors.
This family does not include all known members, as there are problems
with overlapping specificity with pfam00001. This family is greatly
expanded in the nematode worm C. elegans. (SEQ ID NO:811)
CD-Length = 297 residues, 83.8% aligned
Score = 38.1 bits (87), Expect = 0.001

| | | | |
|---|---|---|---|
| NOV 9: | 55 | IFALALFGNALVFYVVTR--SKAMRTVTN---IFICSLALSDLLITFFCIPVTMIQNISD | 109 |
| | | \| ++\| + \|\| + \| \|++\| \|\| \|\| ++\| \|\| ++ | |
| Sbjct: | 16 | ITIISLPIHIFGFYCILFKTPKKMKSVKWSLLNLHFWSALLDLYLSFLTIPYLFFPVLAG | 75 |
| NOV 9: | 110 | NWLEGAFICKMVPFVQSTAVVTEILTMTC----IAVERHQGLVHPFKMKWQYTNRRAFTM | 165 |
| | | \| + +\| \|\| + + + \|\| \|\|+ \|++ | |
| Sbjct: | 76 | YPLGLLSYLGVPTSIQIYIGVTILGVAVSIILLFENSLVNINN-KFRIWKWIRILY | 134 |
| NOV 9: | 166 | LGVVWLVAVIVGSPMWHVQQLEIKYDFLYEKEHICCLEEWTSPVHQKIYTTFILVILFLL | 225 |
| | | \| + +++\|\|+ \|++ + + + \| \|++ \| \|+ + + + + | |
| Sbjct: | 135 | LILNYILAVLFFLPVFLLIPEDQEAAKLKLKKYPCPPPEFFDEPNFFVLAIDSNYFVISI | 194 |
| NOV 9: | 226 | PLMEKKRAVIMMVTVVALFAVCWAPFHMMIEYSNFEKEYDDVTIKMIFAI-VQIIGF | 284 |
| | | + ++++ + + + + + + + 10 + + \| \|+ +\|+ | |
| Sbjct: | 195 | VFLI---LIVILQIIFFVSLIFYYLKILKNSTMSKKTRKLQ-----KKFFIALCIQVSIP | 246 |
| NOV 9: | 285 | SNSICNPIVYAFMNENFK 302 | |
| | | \| \|++\| + \| | |
| Sbjct: | 247 | ILVILIPLIYLVFSIIFG 264 | |

The NOV9 nucleic acids and polypeptides share structure similarity to members to the Neuropeptide Y/Peptide YY/Orexin/Galanin/Cholecystokinin/pancreatic polypeptide receptor family Neuropeptide Y (NPY) is one of the most abundant neuropeptides in the mammalian nervous system and exhibits a diverse range of important physiologic activities, including effects on psychomotor activity, food intake, regulation of central endocrine secretion, and potent vasoactive effects on the cardiovascular system. It shows sequence homology to peptide YY and over 50% homology in amino acid and nucleotide sequence to pancreatic polypeptide. Neuropeptide Y (NPY) signals through a family of G protein-coupled receptors present in the brain and sympathetic neurons. At least 3 types of neuropeptide Y receptor have been defined on the basis of pharmacologic criteria, tissue distribution, and structure of the encoding gene. The NPY Y1 receptors have been identified in a variety of tissues, including brain, spleen, small intestine, kidney, testis, placenta, and aortic smooth muscle. The Y2 receptor is found mainly in the central nervous system.

Orexin A and Orexin B, are derived from the same precursor, orexin, or hypocretin (HCRT), by proteolytic processing. One receptor, designated OX2R, binds both orexin A and orexin B. The predicted amino acid sequences of human and rat OX2R are 95% identical and contain 7 putative transmembrane domains. The other receptor, designated OX1R (HCRTR1), binds orexin A only and has 64% identity to OX2R. Northern blot analysis revealed that in the rat a 3.5-kb OX2R mRNA is expressed exclusively in the brain. When administered intracerebroventricularly to rats, orexin A and orexin B stimulated food consumption. In addition, preproorexin mRNA levels are upregulated upon fasting. thust these peptides are mediators in the central feedback mechanism that regulates feeding behavior.

PYY is secreted from endocrine cells in the lower small intestine, colon, and pancreas. It acts through the pancreatic polypeptide receptors in the gastrointestinal tract as an inhibitor of gastric acid secretion, gastric emptying, digestive enzyme secretion by the pancreas, and gut motility.

The disclosed NOV9 nucleic acid of the invention encoding a Neuropeptide Y/Peptide YY receptor-like protein includes the nucleic acid whose sequence is provided in Table 9A or a fragment thereof. The invention also includes a mutant or variant nucleic acid any of whose bases may be changed from the corresponding base shown in Table 9A while still encoding a protein that maintains its Neuropeptide Y/Peptide YY receptor-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 15 percent of the bases may be so changed.

The disclosed NOV9 protein of the invention includes the Neuropeptide Y/Peptide YY receptor-like protein whose sequence is provided in Table 9B. The invention also includes a mutant or variant protein any of whose residues may be changed from the corresponding residue shown in Table 9B while still encoding a protein that maintains its Neuropeptide Y/Peptide YY receptor-like activities and physiological functions, or a functional fragment thereof. In the mutant or variant protein, up to about 70 percent of the residues may be so changed.

The invention further encompasses antibodies and antibody fragments, such as F_{ab} or (F_{ab})₂, that bind immunospecifically to any of the proteins of the invention.

The above disclosed information suggests that this Neuropeptide Y/Peptide YY receptor-like protein (NOV9) is a member of a “Neuropeptide Y/Peptide YY receptor family”. Therefore, the NOV9 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV9 nucleic acids and proteins of the invention are useful in potential therapeutic applications implicated in obesity, diabetes, kidney disorders, cardiovascular disorders, anorexia, eating disorders, gastrointestinal and digestive diseases, metabolic diseases,CNS disorders, cancer, autoimmune disease, inflammation, and/or other pathologies and disorders.

NOV9 nucleic acids and polypeptides are further useful in the generation of antibodies that bind immuno-specifically to the novel NOV9 substances for use in therapeutic or diagnostic methods. These antibodies may be generated according to methods known in the art, using prediction from hydrophobicity charts, as described in the “Anti-NOVX Antibodies” section below. The disclosed NOV9 protein has multiple hydrophilic regions, each of which can be used as an immunogen. These novel proteins can be used in assay systems for functional analysis of various human disorders, which will help in understanding of pathology of the disease and development of new drug targets for various disorders.

NOV10

A disclosed NOV10 nucleic acid of 985 nucleotides (also referred to as CG55964-01) encoding a novel G-Protein Coupled Receptor-like protein is shown in Table 10A. An open reading frame was identified beginning with an ATG initiation codon at nucleotides 33-35 and ending with a TGA codon at nucleotides 981-983. A putative untranslated region upstream from the initiation codon is underlined in Table 10A. The start and stop codons are in bold letters.

TABLE 10A
NOV10 nucleotide sequence.

(SEQ ID NO:49)

CAAATCTACCACTTGATTCTGATGAACAAATCATGCCGACATTCAATGGCTCAGTCTTCATGCCCTCTGCGT
TTATACTAATTGGGATTCCTGGTCTGGAGTCACTGCAGTGTTGGATTGGGATTCCTTTCTCTGCCATGTATC
TTATTGGTGTGATTGGAAATTCCCTAATTTTAGTTATAATCAAATATGAAAACACCCTCCATATACCCATGT
ACATTTTTTTGGCCATGTTGGCAGCCACAGACATTGCACTTAACACCTGCATTCTTCCCAAAATGTTAGGCA
TCTTCTGGTTTCATTTGCCAGAGATTTCTTTTGATGCCTGTCTTTTTCAAATGTGGCTTATTCACTCATTCC
AGGCAATTGAATCGGGTATCCTTCTGGCAATGGCCCTGGATCGCTATGTGGCCATCTGTATCCCCTTGAGAC
ATGCCACCATCTTTTCCCAGCAGTTCTTAACTCATATTGGACTTGGGGTGACACTCAGGGCTGCCATTCTTA
TAATACCTTCCTTAGGGCTCATCAAATGCTGTCTGAAACACTATCGAACTACAGTCATCTCTCACTCTTACT
GTGAGCACATGGCCATCGTGAAGCTGGCTACTGAAGATATCCGAGTCAACAAGATATATGGCCTATTCGTTG
CCTTTGCAATCCTAGGGTTTGACATAATATTTATAACCTTCTCCTATGTCCAAATTTTTATCACTGTCTTTC
AGCTGCCCCAGAAGGAGGCACGATTCAAGGCCTTTAATACATGCATTGCCCACATTTGTGTCTTCCTACAGT
TCTACCTTCTTCCCTTCTTCTCTTTCTTCACACACAGGTTTGGTTCACACATACCACCATATATTCATATCC
TCTTGTCAAATCTTTACCTGTTAGTCCCACCTTTTCTCAACCCTATTGTCTATGCAGTGAAGACCAAGCAAA
TTCGTGACCATATTGTGAAAGTGTTTTTCTTCAAAAAAGTAACTTGATC

In a search of public sequence databases, the NOV10 nucleic acid sequence has 789 of 974 bases (81%) identical to a gb:GENBANK-ID:AF133300|acc:AF133300.2 mRNA from Mus musculus (MOR 3′Beta1, MOR 3′Beta2, MOR 3′Beta3, and MOR 3′Beta4 genes, complete cds; Cbx3 pseudogene, complete sequence; and MOR 3′Beta5 and MOR 3′Beta6 genes, complete cds) (E=4.3e⁻¹³⁶).

The disclosed NOV10 polypeptide (SEQ ID NO:50) encoded by SEQ ID NO:49 has 316 amino acid residues and is presented in Table 10B using the one-letter amino acid code. Signal P, Psort and/or Hydropathy results predict that NOV10b has a signal peptide and is likely to be localized to the endoplasmic reticulum (membrane) with a certainty of 0.6850. Alternatively, NOV10 may also localize to the plasma membrane with a certainty of 0.6400, the Golgi body with a certainty of 0.4600, or in the endoplasmic reticulum (lumen) with a certainty of 0.1000. The most likely cleavage site for NOV10 is between positions 24 and 25: LES-VQ.

TABLE 10B
Encoded NOV10 protein sequence.

| | |
|---|---|
| MPTFNGSVFMPSAFILIGIPGLESVQCWIGIPFSAMYLIGVIGNSLILVIIKYENSLHIPMYIF | (SEQ ID NO:50) |
| LAMLAATDIALNTCILPKMLGIFWFHLPEISFDACLFQMWLIHSFQAIESGILLAMALDRYVAI | |
| CIPLRHATIFSQQFLTHIGLGVTLRAAILIIPSLGLIKCCLKHYRTTVISHSYCEHMAIVKLAT | |
| EDIRVNKIYGLFVAFAILGFDIIFITLSYVQIFITVFQLPQKEARFKAFNTCIAHICVFLQFYL | |
| LAFFSFFTHRFGSHIPPYIHILLSNLYLLVPPFLNPIVYGVKTKQIRDHIVKVFFFKKVT | |

A search of sequence databases reveals that the NOV10 amino acid sequence has 316 of 316 amino acid residues (100%) identical to, and 316 of 316 amino acid residues (100%) similar to, the 316 amino acid residue ptnr:TREMBLNEW-ACC:AAG42368 protein from Homo sapiens (Human) (Odorant Receptor HOR3′BETA5) (E=5.7e⁻¹⁶⁹).

NOV10 is predicted to be expressed in at least Apical microvilli of the retinal pigment epithelium, arterial (aortic), basal forebrain, brain, Burkitt lymphoma cell lines, corpus callosum, cardiac (atria and ventricle), caudate nucleus, CNS and peripheral tissue, cerebellum, cerebral cortex, colon, cortical neurogenic cells, endothelial (coronary artery and umbilical vein) cells, palate epithelia, eye, neonatal eye, frontal cortex, fetal hematopoietic cells, heart, hippocampus, hypothalamus, leukocytes, liver, fetal liver, lung, lung lymphoma cell lines, fetal lymphoid tissue, adult lymphoid tissue, Those that express MHC II and III nervous, medulla, subthalamic nucleus, ovary, pancreas, pituitary, placenta, pons, prostate, putamen, serum, skeletal muscle, small intestine, smooth muscle (coronary artery in aortic) spinal cord, spleen, stomach, taste receptor cells of the tongue, testis, thalamus, and thymus tissue. This information was derived by determining the tissue sources of the sequences that were included in the invention including but not limited to SeqCalling sources, Public EST sources, Literature sources, and/or RACE sources.

The disclosed NOV10 polypeptide has homology to the amino acid sequences shown in the BLASTP data listed in Table 10C.

TABLE 10C
BLAST results for NOV10

| Gene Index/ | | Length | Identity | Positives | |
|---|---|---|---|---|---|
| Identifier | Protein/Organism | (aa) | (%) | (%) | Expect |
| gi\|11991867\|gb\|AAG42368.1\| | odorant receptor | 316 | 316/316 | 316/316 | e−148 |
| (AF289204) | HOR3'beta5 [Homo | | (100%) | (100%) | |
| | sapiens] | | | | |
| gi\|7305351\|ref\|NP_038648.1\| | olfactory | 315 | 258/314 | 281/314 | e−122 |
| (NM_013620) | receptor 68 [Mus | | (82%) | (89%) | |
| | musculus] | | | | |
| gi\|7305353\|ref\|NP_038649.1\| | olfactory | 316 | 255/314 | 279/314 | e−120 |
| (NM_013621) | receptor 69 [Mus | | (81%) | (88%) | |
| | musculus] | | | | |
| gi\|11908221\|gb\|AAG41685.1\| | MOR 3'Beta6 [Mus | 316 | 238/311 | 268/311 | e−115 |
| (AF133300) | musculus] | | (76%) | (85%) | |
| gi\|6912560\|ref\|NP_036507.1\| | olfactory | 312 | 233/310 | 263/310 | e−110 |
| (NM_012375) | receptor, family | | (75%) | (84%) | |
| | 52, subfamily A, | | | | |
| | member 1 [Homo | | | | |
| | sapiens] | | | | |

The homology between these and other sequences is shown graphically in the ClustalW analysis shown in Table 10D. In the ClustalW alignment of the NOV10 protein, as well as all other ClustalW analyses herein, the black outlined amino acid residues indicate regions of conserved sequence (i.e., regions that may be required to preserve structural or functional properties), whereas non-highlighted amino acid residues are less conserved and can potentially be altered to a much broader extent without altering protein structure or function.

[image]

[image]

Table 10E lists the domain description from DOMAIN analysis results against NOV10. This indicates that the NOV10 sequence has properties similar to those of other proteins known to contain this domain.

TABLE 10E
Domain Analysis of NOV10

gn1|Pfam|pfam00001, 7tm_1, 7 transmembrane receptor (rhodopsin
family). (SEQ ID NO:810)
CD-Length = 254 residues, 100.0% aligned
Score = 67.8 bits (164), Expect = 9e−13

| | | | |
|---|---|---|---|
| NOV10: | 43 | GNSLILVIIKYENSLHIPMYIFLAMLAATDIALNTCILPKMLGIFWFHLPEISFDACLFQ | 102 |
| | | \|\| \|++++\| \| \| \|\|\| \|\| \|+ + \| \| \| | |
| Sbjct: | 1 | GNLLVILVILRTKKLRTPTNIFLLNLAVADLLFLLTLPPWALYYLVGGDWVFGDALCKLV | 60 |
| NOV10: | 103 | MWLIHSFQAIESGILLAMALDRYVAICIPLRHATIFSQQFLTHIGLGVTLRAAILIIPSL | 162 |
| | | \| +\| \|+++\|\|\|+\|\| \|\|\|+ \| + + + \| \| + \| +\| +\| \| | |
| Sbjct: | 61 | GALFVVNGYASILLLTAISIDRYLAIVHPLRYRRIRTPRRAKVLILLVWVLALLLSLPPL | 120 |
| NOV10: | 163 | GLIKCCLKHYR-TTVISHSYCEHMAIVKLATEDIRVNKIYGLFVAFAILGF--DIIFITL | 219 |
| | | \|\|\| + \| ++ + \| + ++ \| \|\| | 219 |
| Sbjct: | 121 | LFSWLRTVEEGNTTVCLIDFPEESVKRSYVL----LSTLVGFLPLLVILVCYTRILRTL | 176 |
| NOV10: | 220 | SYVQIFITVFQLPQKEARFKAFNTCIAHICVFLQF--YLLAFFSFFTHRFGSHIPPYIHI | 277 |
| | | + \| \| + + \| + \| + + | |
| Sbjct: | 177 | RKRARSQRSLKRRSSSERKAAKMLLVVVVVFVLCWLPYHIVLLLDSLCLLSIWRVLPTAL | 236 |
| NOV10: | 278 | LLSNLYLLVPPFLNPIVY 295 | |
| | | \|++ \| \|\|\|\|+\| | |
| Sbjct: | 237 | LITLWLAYVNSCLNPIIY 254 | |

G-Protein Coupled Receptor (GPCRs) have been identified as extremely large subfamily of G protein-coupled receptors in a number of species. These receptors share a seven transmembrane domain structure with many neurotransmitter and hormone receptors, and are likely to underlie the recognition and G-protein-mediated transduction of various signals. Previously, GPCR genes cloned in different species were from random locations in the respective genomes. The human GPCR genes are intron less and belong to four different gene subfamilies, displaying great sequence variability. These genes are dominantly expressed in olfactory epithelium.

Olfactory receptors (ORs) have been identified as extremely large subfamily of G protein-coupled receptors in a number of species. These receptors share a seven transmembrane domain structure with many neurotransmitter and hormone receptors, and are likely to underlie the recognition and G-protein-mediated transduction of odorant signals. Previously, OR genes cloned in different species were from random locations in the respective genomes. The human OR genes are intron less and belong to four different gene subfamilies, displaying great sequence variability. These genes are dominantly expressed in olfactory epithelium.

The disclosed NOV10 nucleic acid of the invention encoding a G-Protein Coupled Receptor-like protein includes the nucleic acid whose sequence is provided in Table 10A or a fragment thereof. The invention also includes a mutant or variant nucleic acid any of whose bases may be changed from the corresponding base shown in Table 10A while still encoding a protein that maintains its G-Protein Coupled Receptor-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 19 percent of the bases may be so changed.

The disclosed NOV10 protein of the invention includes the G-Protein Coupled Receptor-like protein whose sequence is provided in Table 10B. The invention also includes a mutant or variant protein any of whose residues may be changed from the corresponding residue shown in Table 10B while still encoding a protein that maintains its G-Protein Coupled Receptor-like activities and physiological functions, or a functional fragment thereof. In the mutant or variant protein, up to about 25 percent of the residues may be so changed.

The invention further encompasses antibodies and antibody fragments, such as F_{ab} or (F_{ab})₂, that bind immunospecifically to any of the proteins of the invention.

The above disclosed information suggests that this G-Protein Coupled Receptor-like protein (NOV10) is a member of a “G-Protein Coupled Receptor family”. Therefore, the NOV10 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV10 nucleic acids and proteins of the invention are useful in potential therapeutic applications implicated in developmental diseases, MHCII and III diseases (immune diseases), Taste and scent detectability Disorders, Burkitt's lymphoma, Corticoneurogenic disease, Signal Transduction pathway disorders, Retinal diseases including those involving photoreception, Cell Growth rate disorders; Cell Shape disorders, Feeding disorders;control of feeding; potential obesity due to over-eating; potential disorders due to starvation (lack of apetite), noninsulin-dependent diabetes mellitus (NIDDM1), bacterial, fungal, protozoal and viral infections (particularly infections caused by HIV-1 or HIV-2), pain, cancer (including but not limited to Neoplasm; adenocarcinoma; lymphoma; prostate cancer; uterus cancer), anorexia, bulimia, asthma, Parkinson's disease, acute heart failure, hypotension, hypertension, urinary retention, osteoporosis, Crohn's disease; multiple sclerosis; and Treatment of Albright Hereditary Ostoeodystrophy, angina pectoris, myocardial infarction, ulcers, asthma, allergies, benign prostatic hypertrophy, and psychotic and neurological disorders, including anxiety, schizophrenia, manic depression, delirium, dementia, severe mental retardation. Dentatorubro-pallidoluysian atrophy(DRPLA) Hypophosphatemic rickets, autosomal dominant (2) Acrocallosal syndrome and dyskinesias, such as Huntington's disease or Gilles de la Tourette syndrome, and/or other diseases and pathologies.

NOV10 nucleic acids and polypeptides are further useful in the generation of antibodies that bind immuno-specifically to the novel NOV10 substances for use in therapeutic or diagnostic methods. These antibodies may be generated according to methods known in the art, using prediction from hydrophobicity charts, as described in the “Anti-NOVX Antibodies” section below. The disclosed NOV10 protein has multiple hydrophilic regions, each of which can be used as an immunogen. These novel proteins can be used in assay systems for functional analysis of various human disorders, which will help in understanding of pathology of the disease and development of new drug targets for various disorders.

NOV11

A disclosed NOV11 nucleic acid of 1014 nucleotides (also referred to as Curagen Accession No. CG55966-01) encoding a novel G-Protein Coupled Receptor-like protein is shown in Table 11A. An open reading frame was identified beginning with an ATG initiation codon at nucleotides 2-4 and ending with a TGA codon at nucleotides 947-949. Putative untranslated regions upstream from the initiation codon and downstream of the termination codon are underlined in Table 11A. The start and stop codons are in bold letters.

TABLE 11A
NOV11 nucleotide sequence.

| | |
|---|---|
| AATGATTACTTCAGTAAGCCCTAGCACCAGCACGAATTCTTCCTTTCTTCTCACTGGATTTTCTG | (SEQ ID NO:51) |
| GCATCGAGCAGCAATACCCCTGGTTTTCCATCCCCTTCTCCTCAATCTATCCCATGGTGCTTTTG | |
| GGCAATTGCATGCTTCTCCATGTGATATGGACTGAGCCAAGCCTGCACCAGCCTATGTTTTACTT | |
| CCTGTCCATGCTGGCCCTCACTGACCTGTGCATGCCGCTGTCCACTGTGTACACAGTGCTGGGGA | |
| TCCTGTGGCCGATCATTCGAGAGATCAGCTTGGATTCCTGCATTGCCCAGTCCTATTTCATCCAT | |
| GGTCTGTCCTTCATGGAGTCCTCTGTCCTCCTCACTATGGCCTTTGACCGGTACATTGCAATTTG | |
| CAATCCACTACGTTATTCCTCCATCCTGACTAATTCCAGAATTATCAAAATTGGGCTCACTATAA | |
| TAGGTAGGAGTTTTTTCTTTATTACACCCCCCATCATCTGTCTGAAATTTTTTAATTACTGTCAT | |
| TTCCACATCCTTTCTCACTCTTTCTGCCTGCACCAGGATCTTCTCCGCTTAGCCTGTTCAGACAT | |
| CCGATTCAATAGTTACTATGCCCTGATGCTGGTTATTTGCATACTGTTGTTGGATGCTATACTCA | |
| TCCTTTTCTCCTACATCCTGATTCTTAACTCAGTCCTGCCAGTTGCCTCTCAGGAAGAGACGCAT | |
| AAATTATTTCAGACCTGCATCTCCCACATCTGTGCTGTCCTTGTGTTCTACATCCCTATCATTAG | |
| CCTCACAATGGTGCACCGTTTTGGCAAGCACCTTTCCCCCGTGGCCCACGTTCTCATTGGCAACA | |
| TCTACATCCTTTTCCCACCTTTAATGAATCCCATCATCTACAGTGTCAAGACCCAACACATTCAT | |
| ACCAGAATGCTTAGACTCTTTTCTCTGAAAAGATATTGAGAGATATTGAGATGTATTGCCTAAAA | |
| AAAAGAAAGAAAACCACCAACAATAATAAACAAAAATCA | |

The disclosed NOV11 polypeptide (SEQ ID NO:52) encoded by SEQ ID NO:51 has 315 amino acid residues and is presented in Table 11B using the one-letter amino acid code.

TABLE 11B
Encoded NOV11 protein sequence.

| | |
|---|---|
| MITSVSPSTSTNSSFLLTGFSGMEQQYPWFSIPFSSIYAMVLLGNCMVLHVIWTEPSLHQPMFY | (SEQ ID NO:52) |
| FLSMLALTDLCMGLSTVYTVLGILWRIIREISLDSCIAQSYFIHGLSFMESSVLLTMAFDRYIA | |
| ICNPLRYSSILTNSRIIKIGLTIIGRSFFFITPPIICLKFFNYCHFHILSHSFCLHQDLLRLAC | |
| SDIRFNSYYALMLVICILLLDAILILFSYILILKSVLAVASQEERHKLFQTCISHICAVLVFYI | |
| PIISLTMVHRFGKHLSPVAHVLIGNIYILFPPLMNPIIYSVKTQQIHTRMLRLFSLKRY | |

A search of sequence databases reveals that the NOV11 amino acid sequence has 165 of 302 amino acid residues (54%) identical to, and 222 of 302 amino acid residues (73%) similar to, the 311 amino acid residue ptnr: SPTREMBL-ACC:Q9WVN4 protein from Mus musculus (Mouse) MOR 5′BETA1 (E=7.0e⁻⁸⁸).

The disclosed NOV11 polypeptide has homology to the amino acid sequences shown in the BLASTP data listed in Table 11C.

TABLE 11C
BLAST results for NOV11

| Gene Index/ | | Length | Identity | Positives | |
|---|---|---|---|---|---|
| Identifier | Protein/Organism | (aa) | (%) | (%) | Expect |
| gi\|11991863\|gb\|AAG42364.1\| | odorant receptor | 321 | 315/315 | 315/315 | e−139 |
| (AF289204) | HOR3'beta1 [Homo | | (100%) | (100%) | |
| | sapiens] | | | | |
| gi\|11908218\|gb\|AAG41683.1\| | HOR5'Beta5 [Homo | 312 | 165/307 | 231/307 | 4e−78 |
| (AF137396) | sapiens] | | (53%) | (74%) | |
| gi\|17456753\|ref\|XP_{—} | similar to MOR | 315 | 163/307 | 223/307 | 1e−77 |
| 061614.1\| | 3Beta4 (H. | | (53%) | (72%) | |
| (XM_061614) | sapiens) [Homo | | | | |
| | sapiens] | | | | |
| gi\|7305345\|ref\|NP_038645.1\| | olfactory | 307 | 164/305 | 223/305 | 5e−77 |
| (NM_013617) | receptor 65 [Mus | | (53%) | (72%) | |
| | musculus] | | | | |
| gi\|17456767\|ref\|XP_{—} | similar to | 879 | 162/303 | 226/303 | 2e−76 |
| 061618.1\| | prostate specific | | (53%) | (74%) | |
| (XM_061618) | G-protein coupled | | | | |
| | receptor (H. | | | | |
| | sapiens) [Homo | | | | |
| | sapiens] | | | | |

The homology between these and other sequences is shown graphically in the ClustalW analysis shown in Table 11D. In the ClustalW alignment of the NOV11 protein, as well as all other ClustalW analyses herein, the black outlined amino acid residues indicate regions of conserved sequence (i.e., regions that may be required to preserve structural or functional properties), whereas non-highlighted amino acid residues are less conserved and can potentially be altered to a much broader extent without altering protein structure or function.

[image]

[image]

[image]

Table 11E lists the domain description from DOMAIN analysis results against NOV11. This indicates that the NOV11 sequence has properties similar to those of other proteins known to contain this domain.

TABLE 11E
Domain Analysis of NOV11

gn1|Pfam|pfam00001, 7tm_1, 7 transmembrane receptor (rhodopsin
family). (SEQ ID NO:810)
CD-Length = 254 residues, 100.0% aligned
Score = 71.2 bits (173), Expect = 8e−14

| | | | |
|---|---|---|---|
| NOV11: | 44 | GNCMVLHVIWTEPSLHQPMFYFLSMLALTDLCMGLSTVYTVLGILWRIIREISLDSCIAQ | 103 |
| | | \|\| +\|+ \|\| \| \| \|\| \|\|+ \|\| \|+ \| \| \| | |
| Sbjct: | 1 | GNLLVILVILRTKKLRTPTNIFLLNLAVADLLFLLTLPPWALYYLVGGDWVFGDALCKLV | 60 |
| NOV11: | 104 | SYFIHGLSFMESSVLLTMAFDRYIAICNPLRYSSILTNSRIIKIGLTIIGRSFFFITPPI | 163 |
| | | + +\| ++ \|\|\|+\|\| +\|\|\|\| \| \| \| + \| + + \|\|+ | |
| Sbjct: | 61 | GALFVVNGYASILLLTAISIDRYLAIVHPLRYRRIRTPRRAKVLILLVWVLALLLSLPPL | 120 |
| NOV11: | 164 | ICLKFFNYCHFHILSHSFCLHQDLLRLACSDIRFNSYYALMLVICILLLDAILILFSYIL | 223 |
| | | + \| + + \|\| + \| \|+ + +\| ++\|\| \| | |
| Sbjct: | 121 | L---FSWLRTVEEGNTTVCLIDF------PEESVKRSYVLLSTLVGFVLPLLVILVCYTR | 171 |
| NOV11: | 224 | ILKSVLAVA---------SQEERHKLFQTCISHICAVLVF--YIPIISLTMVHRFGKHLS | 272 |
| | | \|\|+++ \| \| \|\| + + \|\| + \| ++ \| + | |
| Sbjct: | 172 | ILRTLRKRARSQRSLKRRSSSERKAAKMLLVVVVVFVLCWLPYHIVLLLDSLCLLSIWRV | 231 |
| NOV11: | 273 | PVAHVLIGNIYILFPPLMNPIIY 295 | |
| | | +\|\| +\|\|\|\|\| | |
| Sbjct: | 232 | LPTALLITLWLAYVNSCLNPIIY 254 | |

G-Protein Coupled Receptor (GPCRs) have been identified as extremely large subfamily of G protein-coupled receptors in a number of species. These receptors share a seven transmembrane domain structure with many neurotransmitter and hormone receptors, and are likely to underlie the recognition and G-protein-mediated transduction of various signals. Previously, GPCR genes cloned in different species were from random locations in the respective genomes. The human GPCR genes are intron less and belong to four different gene subfamilies, displaying great sequence variability. These genes are dominantly expressed in olfactory epithelium.

Olfactory receptors (ORs) have been identified as extremely large subfamily of G protein-coupled receptors in a number of species. These receptors share a seven transmembrane domain structure with many neurotransmitter and hormone receptors, and are likely to underlie the recognition and G-protein-mediated transduction of odorant signals. Previously, OR genes cloned in different species were from random locations in the respective genomes. The human OR genes are intron less and belong to four different gene subfamilies, displaying great sequence variability. These genes are dominantly expressed in olfactory epithelium.

The disclosed NOV11 nucleic acid of the invention encoding a G-Protein Coupled Receptor-like protein includes the nucleic acid whose sequence is provided in Table 11A or a fragment thereof. The invention also includes a mutant or variant nucleic acid any of whose bases may be changed from the corresponding base shown in Table 11A while still encoding a protein that maintains its G-Protein Coupled Receptor-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

The disclosed NOV11 protein of the invention includes the G-Protein Coupled Receptor-like protein whose sequence is provided in Table 11B. The invention also includes a mutant or variant protein any of whose residues may be changed from the corresponding residue shown in Table 11B while still encoding a protein that maintains its G-Protein Coupled Receptor-like activities and physiological functions, or a functional fragment thereof. In the mutant or variant protein, up to about 47 percent of the residues may be so changed.

The invention further encompasses antibodies and antibody fragments, such as F_{ab} or (F_{ab})₂, that bind immunospecifically to any of the proteins of the invention.

The above disclosed information suggests that this G-Protein Coupled Receptor-like protein (NOV11) is a member of a “G-Protein Coupled Receptor family”. Therefore, the NOV11 nucleic acids and proteins identified here may be useful in potential therapeutic applications implicated in (but not limited to) various pathologies and disorders. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV11 nucleic acids and proteins of the invention are useful in potential therapeutic applications implicated in developmental diseases, MHCII and III diseases (immune diseases), Taste and scent detectability Disorders, Burkitt's lymphoma, Corticoneurogenic disease, Signal Transduction pathway disorders, Retinal diseases including those involving photoreception, Cell Growth rate disorders; Cell Shape disorders, Feeding disorders;control of feeding; potential obesity due to over-eating; potential disorders due to starvation (lack of apetite), noninsulin-dependent diabetes mellitus (NIDDM1), bacterial, fungal, protozoal and viral infections (particularly infections caused by HIV-1 or HIV-2), pain, cancer (including but not limited to Neoplasm; adenocarcinoma; lymphoma; prostate cancer; uterus cancer), anorexia, bulimia, asthma, Parkinson's disease, acute heart failure, hypotension, hypertension, urinary retention, osteoporosis, Crohn's disease; multiple sclerosis; and Treatment of Albright Hereditary Ostoeodystrophy, angina pectoris, myocardial infarction, ulcers, asthma, allergies, benign prostatic hypertrophy, and psychotic and neurological disorders, including anxiety, schizophrenia, manic depression, delirium, dementia, severe mental retardation. Dentatorubro-pallidoluysian atrophy(DRPLA) Hypophosphatemic rickets, autosomal dominant (2) Acrocallosal syndrome and dyskinesias, such as Huntington's disease or Gilles de la Tourette syndrome, and/or other pathologies and disorders.

NOV11 nucleic acids and polypeptides are further useful in the generation of antibodies that bind immuno-specifically to the novel NOV11 substances for use in therapeutic or diagnostic methods. These antibodies may be generated according to methods known in the art, using prediction from hydrophobicity charts, as described in the “Anti-NOVX Antibodies” section below. These novel proteins can be used in assay systems for functional analysis of various human disorders, which will help in understanding of pathology of the disease and development of new drug targets for various disorders.

NOV12

A disclosed NOV12 nucleic acid of 1067 nucleotides (also referred to as Curagen Accession No. CG56003-01) encoding a novel G-Protein Coupled Receptor-like protein is shown in Table 12A. An open reading frame was identified beginning with an ATG initiation codon at nucleotides 15-17 and ending with a TGA codon at nucleotides 1023-1025. The untranslated regions are underlined and the start and stop codons are in bold letters in Table 12A.

TABLE 12A
NOV12 nucleotide sequence.

(SEQ ID NO:53)

AAAACCTGACATAAATGAACAACAATACAACATCTATTCAACCATCTATGATCTCTTCCATGGCTTTACCAA
TCATTTACATCCTCCTTTGTATTGTTGGTGTTTTTGGAAACACTCTCTCTCAATGGATATTTTTAACAAAAA
TAGGTAAAAAAACATCAACGCACATCTACCTGTCACACCTTGTGACTGCAAACTTACTTGTGTGCAGTGCCA
TGCCTTTCATGAGTATCTATTTCCTGAAAGGTTTCCAATGGGAATATCAATCTGCTCAATGCAGAGTGGTCA
ATTTTCTGGGAACTCTATCCATGCATGCAAGTATGTTTGTCAGTCTCTTAATTTTAAGTTGGATTGCCATAA
GCCCCTATGCTACCTTAATGCAAAAGGATTCCTCGCAAGAGACTACTTCATGCTATGAGAAAATATTTTATG
GCCATTTACTGAAAAAATTTCGCCAGCCCAACTTTGCTAGAAAACTATGCATTTACATATGGGCAGTTGTAC
TGGGCATAATCATTCCAGTTACCGTATACTACTCAGTCATAGAGGCTACACAAGGAGAAGAGAGCCTATGCT
ACAATCGGCAGATGGAACTAGGAGCCATGATCTCTCAGATTGCAGGTCTCATTGGAACCACATTTATTGGAT
TTTCCTTTTTAGTAGTACTAACATCATACTACTCTTTTGTAAGCCATCTGAGAAAAATAACAACCTGTACGT
CCATTATGGAGAAAGATTTGACTTACACTTCTGTGAAAAGACATCTTTTGGTCATCCAGATTCTACTAATAG
TTTGCTTCCTTCCTTATAGTATTTTTAAACCCATTTTTTATGTTCTACACCAAAGAGATAACTGTCAGCAAT
TGAATTATTTAATAGAAACAAAAAACATTCTCACCTGTCTTGCTTCGGCCAGAAGTAGCACAGACCCCATTA
TATTTCTTTTATTAGATAAAACATTCAAGAAGACACTATATAATCTCTTTACAAAGTCTAATTCAGCACATA
TGCAATCATATGGTTGACTTTTGAATGGAAAACCCCACAATATTAAGAAAAGCATTCAT

The disclosed NOV12 polypeptide (SEQ ID NO 54) encoded by SEQ ID NO:53 has 336 amino acid residues and is presented in Table 12B using the one-letter amino acid code.

TABLE 12B
Encoded NOV12 protein sequence.

(SEQ ID NO:54)

MNNNTTCIQPSMISSMALPIIYILLCIVGVFGNTLSQWIFLTKIGKKTSTHIYLSHLVTANLLV
CSAMPFMSIYFLKGFQWEYQSAQCRVVNFLGTLSMHASMFVSLLILSWIAISRYATLMQKDSSQ
ETTSCYEKIFYGHLLKKFRQPNFARKLCIYIWGVVLGIIIPVTVYYSVIEATEGEESLCYNRQM
ELGAMISQIAGLIGTTFIGFSFLVVLTSYYSFVSHLRKIRTCTSIMEKDLTYSSVRHLLVIQI
LLIVCFLPYSIFKPIFYVLHQRDNCQQLNYLIETKNILTCLASARSSTDPIIFLLLDKTFKKTLYNLFT
KSNSAHMQSYG

A search of sequence databases reveals that the NOV12 amino acid sequence has 52 of 179 amino acid residues (29%) identical to, and 86 of 179 amino acid residues (48%) similar to, the 339 amino acid residue ptnr: SWISSPROT-ACC:Q13304 protein from Homo sapiens Putative G Protein-Coupled Receptor GPR17 (R12) (E=1.6e⁻²²).

G-Protein Coupled Receptor (GPCRs) have been identified as extremely large subfamily of G protein-coupled receptors in a number of species. These receptors share a seven transmembrane domain structure with many neurotransmitter and hormone receptors, and are likely to underlie the recognition and G-protein-mediated transduction of various signals. Previously, GPCR genes cloned in different species were from random locations in the respective genomes. The human GPCR genes are intron less and belong to four different gene subfamilies, displaying great sequence variability. These genes are dominantly expressed in olfactory epithelium.

The disclosed NOV12 polypeptide has homology to the amino acid sequences shown in the BLASTP data listed in Table 12C.

TABLE 12C
BLAST results for NOV12

| Gene Index/ | | Length | Identity | Positives | |
|---|---|---|---|---|---|
| Identifier | Protein/Organism | (aa) | (%) | (%) | Expect |
| gi\|18201870\|ref\|NP_{—} | G protein-coupled | 336 | 336/336 | 336/336 | e−170 |
| 543007.1\| | receptor 82 [Homo | | (100%) | (100%) | |
| (NM_080817) | sapiens] | | | | |
| gi\|4885301\|ref\|NP_005282.1\| | G protein-coupled | 367 | 85/322 | 144/322 | 6e−21 |
| (NM_005291) | receptor 17 [Homo | | (26%) | (44%) | |
| | sapiens] | | | | |
| gi\|17462169\|ref\|XP_{—} | G protein-coupled | 339 | 85/322 | 144/322 | 2e−20 |
| 002705.4\| | receptor 17 [Homo | | (26%) | (44%) | |
| (XM_002705) | sapiens] | | | | |
| gi\|2695876\|emb\|CAB08108.1\| | P2Y-like G- | 298 | 80/302 | 135/302 | 3e−18 |
| (Z94155) | protein coupled | | (26%) | (44%) | |
| | receptor [Homo | | | | |
| | sapiens] | | | | |
| gi\|5757634\|gb\|AAD50531.1\| | G-protein coupled | 381 | 77/323 | 152/323 | 4e−18 |
| AF039686_1 | receptor GPR34 | | (23%) | (46%) | |
| (AF039686) | [Homo sapiens] | | | | |

The homology between these and other sequences is shown graphically in the ClustalW analysis shown in Table 12D. In the ClustalW alignment of the NOV12 protein, as well as all other ClustalW analyses herein, the black outlined amino acid residues indicate regions of conserved sequence (i.e., regions that may be required to preserve structural or functional properties), whereas non-highlighted amino acid residues are less conserved and can potentially be altered to a much broader extent without altering protein structure or function.

[image]

[image]

Table 12E lists the domain description from DOMAIN analysis results against NOV12. This indicates that the NOV12 sequence has properties similar to those of other proteins known to contain this domain.

TABLE 12E
Domain Analysis of NOV12

gn1|Pfam|pfam00001, 7tm_1, 7 transmembrane receptor (rhodopsin
family). (SEQ ID NO:810)
CD-Length = 254 residues, 99.6% aligned
Score = 82.0 bits (201), Expect = 5e−17

| | | | |
|---|---|---|---|
| NOV12: | 32 | GNTLSQWIFLTKIGKKTSTHIYLSHLVTANLLVCSAMPFMSIYFLKGFQWEYQSAQCRVV | 91 |
| | | \|\| \| + \| +\| \|+\|+\| +\| \|+\|\| +\| ++\|+\| \| \| + \| \|++\| | |
| Sbjct: | 1 | GNLLVILVILRTKKLRTPTNIFLLNLAVADLLFLLTLPPWALYYLVGGDWVFGDALCKLV | 60 |
| NOV12: | 92 | NFLGTLSMHASMFVSLLILSWIAISRYATLMQKDSSQETTSCYEKIFYGHLLKKFRQPNF | 151 |
| | | \| ++ +\|\|+ +\|+ \|+\| \|\| \| + ++ \| \| | |
| Sbjct: | 61 | GALFVVNGYASIL----LLTAISIDRYLA----------------IVHPLRYRRIRTPRR | 100 |
| NOV12: | 152 | ARKLCIYIWGVVLGIIIPVTVYYSVIEATEGEESLCYNRQMELGAMISQIAGLIGTTFIG | 211 |
| | | \|+ \| + +\| + \| + +\| ++ + \|\| ++\| \| \| + \|+ | |
| Sbjct: | 101 | AKVLILLVWVLALLLSLPPLLFSWLRTVEEGNTTVCLIDFPEESVKRSYVLLSTLVGFV- | 159 |
| NOV12: | 212 | FSFLVVLTSYYSFVSHLRK-IRTCTSIMEKDLTYSSVKRHLLVIQILLIVCFLPYSIFKP | 270 |
| | | \|\|+\| \| + \|\|\| \|+ \|+ + + + \|\|\|+ ++ ++\|+\|\|\| \| | |
| Sbjct: | 160 | LPLLVILVCYTRILRTLRKIkARSQRSLKRRSSSERKJAAKJVILLVVVVVFVLCWLPYHIVLL | 219 |
| NOV12: | 271 | IFYVLHQRDNCQQLNYLIETKNILTCLASARSSTDPII 308 | |
| | | + + \| \|\| \| +\|\|\| | |
| Sbjct: | 220 | LDSLCLLSIWRVLPT----ALLITLWLAYVNSCLNPII 253 | |

Olfactory receptors (ORs) have been identified as extremely large subfamily of G protein-coupled receptors in a number of species. These receptors share a seven transmembrane domain structure with many neurotransmitter and hormone receptors, and are likely to underlie the recognition and G-protein-mediated transduction of odorant signals. Previously, OR genes cloned in different species were from random locations in the respective genomes. The human OR genes are intron less and belong to four different gene subfamilies, displaying great sequence variability. These genes are dominantly expressed in olfactory epithelium.

The disclosed NOV12 nucleic acid of the invention encoding a G-Protein Coupled Receptor-like protein includes the nucleic acid whose sequence is provided in Table 12A or a fragment thereof. The invention also includes a mutant or variant nucleic acid any of whose bases may be changed from the corresponding base shown in Table 12A while still encoding a protein that maintains its G-Protein Coupled Receptor-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

The disclosed NOV12 protein of the invention includes the G-Protein Coupled Receptor-like protein whose sequence is provided in Table 12B. The invention also includes a mutant or variant protein any of whose residues may be changed from the corresponding residue shown in Table 12B while still encoding a protein that maintains its G-Protein Coupled Receptor-like activities and physiological functions, or a functional fragment thereof. In the mutant or variant protein, up to about 77 percent of the residues may be so changed.

The invention further encompasses antibodies and antibody fragments, such as F_{ab} or (F_{ab})₂, that bind immunospecifically to any of the proteins of the invention.

The above disclosed information suggests that this G-Protein Coupled Receptor-like protein (NOV12) is a member of a “G-Protein Coupled Receptor family”. Therefore, the NOV12 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV12 nucleic acids and proteins of the invention are useful in potential therapeutic applications implicated in developmental diseases, MHCII and III diseases (immune diseases), Taste and scent detectability Disorders, Burkitt's lymphoma, Corticoneurogenic disease, Signal Transduction pathway disorders, Retinal diseases including those involving photoreception, Cell Growth rate disorders; Cell Shape disorders, Feeding disorders;control of feeding; potential obesity due to over-eating; potential disorders due to starvation (lack of apetite), noninsulin-dependent diabetes mellitus (NIDDM1), bacterial, fungal, protozoal and viral infections (particularly infections caused by HIV-1 or HIV-2), pain, cancer (including but not limited to Neoplasm; adenocarcinoma; lymphoma; prostate cancer; uterus cancer), anorexia, bulimia, asthma, Parkinson's disease, acute heart failure, hypotension, hypertension, urinary retention, osteoporosis, Crohn's disease; multiple sclerosis; and Treatment of Albright Hereditary Ostocodystrophy, angina pectoris, myocardial infarction, ulcers, asthma, allergies, benign prostatic hypertrophy, and psychotic and neurological disorders, including anxiety, schizophrenia, manic depression, delirium, dementia, severe mental retardation. Dentatorubro-pallidoluysian atrophy(DRPLA) Hypophosphatemic rickets, autosomal dominant (2) Acrocallosal syndrome and dyskinesias, such as Huntington's disease or Gilles de la Tourette syndrome, and/or other pathologies and disorders.

NOV12 nucleic acids and polypeptides are further useful in the generation of antibodies that bind immuno-specifically to the novel NOV12 substances for use in therapeutic or diagnostic methods. These antibodies may be generated according to methods known in the art, using prediction from hydrophobicity charts, as described in the “Anti-NOVX Antibodies” section below. The disclosed NOV12 protein has multiple hydrophilic regions, each of which can be used as an immunogen. These novel proteins can be used in assay systems for functional analysis, of various human disorders, which will help in understanding of pathology of the disease and development of new drug targets for various disorders.

NOV13

NOV13 includes three novel G-Protein Coupled Receptor-like proteins disclosed below. The disclosed sequences have been named NOV13a and NOV13b.

NOV13a

A disclosed NOV13a nucleic acid of 961 nucleotides (also referred to as CG56075-01) encoding a novel G-Protein Coupled Receptor-like protein is shown in Table 13A. An open reading frame was identified beginning with an ATG initiation codon at nucleotides 12-14 and ending with a TGA codon at nucleotides 936-938. The start and stop codons are shown in bold in Table 13A, and the 5′ and 3′ untranslated regions, if any, are underlined.

TABLE 13A
NOV13a nucleotide sequence.

(SEQ ID NO:55)

GACAACAAACTATGAGACAGATAAATCAGACACAAGTGACAGAATTCCTCCTTCTGGGACTCTCTGATGGGC
CACACACCGAGCAGCTGCTATTTATCGTATTATTGGGTGTCTACCTGGTCACTGTGCTTGGAAATCTGCTTC
TAATCTCCCTTGTTCATGTTGACTCCCAACTTCACACACCCATGTATTTTTTTCTCTGCAACTTGTCTCTGG
CTGACCTCTGTTTCTCTACCAACATAGTTCCTCAGGCACTAGTCCACCTGCTTTCCAGAAAGAAGGTCATTG
CATTCACACTTTGCGCAGCTCGACTTCTCTTTTTCCTCATTTTTGGGTGTACCCAGTGCGCCCTTCTTGCAG
TGATGTCCTATGATCGCTATGTTGCAATCTGCAATCCTCTGCGTTACCCTAACATCATGACCTGGAAAGTGT
GTGTCCAGCTGGCAACAGGATCATGGACCAGTGGCATTCTGGTGTCTGTGGTAGACACCACCTTCACACTGA
GGCTACCCTACCGAGGCAGTAACAGCATTGCTCATTTCTTTTGTGAGGCCCCTGCACTATTGATCTTAGCAT
CCACAGACACCCATGCATCAGAGATGGCCATTTTTCTTACGGGGGTTGTGATTCTCCTCATACCTGTTTTTC
TGATTCTGGTATCCTATGGCCGTATCATAGTAACTGTGGTCAAGATGAAGTCAACTGTGGGGAGTCTCAAGG
CATTTTCTACCTGTGGCTCCCACCTCATGGTGGTCATACTTTTTTATGGATCAGCAATTATCACTTACATGA
CACCCAAGTCTTCCAAACAGCAGGAAAAATCGGTGTCTGTTTTCTATGCAATAGTGACTCCCATGCTTAATC
CCCTCATCTATAGCCTGAGAAACAAGGATGTGAAGGCAGCTCTGAGGAAAGTAGCCACAAGGAATTTCCCAT
GAAGGCTTGGAATCTCACACTGACA

The disclosed NOV13a polypeptide (SEQ ID NO:56) encoded by SEQ ID NO:55 has 308 amino acid residues and is presented in Table 13B using the one-letter amino acid code.

TABLE 13B
Encoded NOV13a protein sequence.

| | |
|---|---|
| MRQINQTQVTEFLLLGLSDGPHTEQLLFIVLLGVYLVTVLGNLLLISLVHVDSQLHTPMYFFLC | (SEQ ID NO:56) |
| NLSLADLCFSTNIVPQALVHLLSRKKKVIAFTLCAARLLFFLIFGCTQCALLAVMSYDRYVAICN | |
| PLRYPNIMTWKVCVQLATGSWTSGILVSVVDTTFTLRLPYRGSNSIAHFFCEAPALLILASTDT | |
| HASEMAIFLTGVVILLIPVFLILVSYGRIIVTVVKMKSTVGSLKAFSTCGSHLMVVILFYGSAI | |
| ITYMTPKSSKQQEKSVSVFYAIVTPMLNPLIYSLRNKDVKAALRKVATRNFP | |

A search of sequence databases reveals that the NOV13a amino acid sequence has 216 of 217 amino acid residues (99%) identical to, and 217 of 217 amino acid residues (100%) similar to, the 217 amino acid residue ptnr: SPTREMBL-ACC:O95224 protein from Homo sapiens (Human) (Olfactory Receptor) (E=2.2e⁻¹⁰⁹).

NOV13b

A disclosed NOV13b nucleic acid of 961 nucleotides (also referred to as CG56021-02) encoding a novel G-Protein Coupled Receptor-like protein is shown in Table 13C. An open reading frame was identified beginning with an ATG initiation codon at nucleotides 12-14 and ending with a TGA codon at nucleotides 936-938. A putative untranslated region upstream from the initiation codon is underlined in Table 13C. The start and stop codons are in bold letters.

TABLE 13C
NOV13b nucleotide sequence.

(SEQ ID NO:57)

GACAACAAACTATGAGACAGATAAATCAGACACAAGTGACAGAATTCCTCCTTCTGGGACTCTGTGATGGGC
CACACACCGAGCAGCTGCTATTTATCGTATTATTGGGTGTCTACCTGGTCACTGTGCTTGGAAATCTGCTTC
TAATCTCCCTTGTTCATGTTGACTCCCAACTTCACACACCCATGTATTTTTTTCTCTGCAACTTGTCTCTGG
CTGACCTCTGTTTCTCTACCAACATAGTTCCTCAGGCACTAATCCACCTGCTTTCCAGAAAGAAGGTCATTG
CATTCACACTTTGCGCAGCTCGACTTCTCTTTTTCCTCATTTTTGGGTGTACCCAGTGCGCCCTTCTTGCAG
TGATGTCCTATGATCGCTATGTTGCAATCTGCAATCCTCTGCGTTACCCTAACATCATGACCTGGAAAGTGT
GTGTCCAGCTGGCAACAGGATCATGGACCAGTGGCATTCTGGTGTCTGTGGTAGACACCACCTTCACACTGA
GGCTACCCTACCGAGGCAGTAACAGCATTGCTCATTTCTTTTGTCAGGCCCCTGCACTATTGATCTTAGCAT
CCACAGACACCCATGCATCAGAGATGGCCATTTTTCTTATGGGGGTTGTGATTCTCCTCATACCTGTTTTTC
TGATTCTGGTATCCTATGGCCGTATCATAGTAACTGTGGTCAAGATGAAGTCAACTGTGGGGAGTCTCAAGG
CATTTTCTACCTGTGGCTCCCACCTCATGGTGGTCATACTTTTTTATGGATCAGCAATTATCACTTGCATGA
CACCCAAGTCTTCCAAACAGCAGGAAAAATCGGTGTCTGTTTTCTATGCAATAGTGACTCCCATGCTTAATC
CCCTCATCTATAGCCTGAGAAACAAGGATGTGAAGGCAGCTCTGAGGAAAGTAGCCACAAGGAATTTCCCAT
GAAGGCTTGGAATCTCACACTGACA

In a search of public sequence databases, the NOV13b nucleic acid sequence has 648 of 653 bases (99%) identical to a gb:GENBANK-ID:AF065876|acc:AF065876.1 mRNA from Homo sapiens (olfactory receptor (OR2D2) gene, partial cds) (E=2.8e⁻¹³⁹).

The disclosed NOV13b polypeptide (SEQ ID NO:58) encoded by SEQ ID NO:57 has 308 amino acid residues and is presented in Table 13D using the one-letter amino acid code. Signal P, Psort and/or Hydropathy results predict that NOV13b has a signal peptide and is likely to be localized to the plasma membrane with a certainty of 0.6000. Alternatively, NOV13b may also localize to the Golgi body with a certainty of 0.4000, the endoplasmic reticulum (membrane) with a certainty of 0.3000, or in the microbody (peroxisome) with a certainty of 0.3000. The most likely cleavage site for NOV13b is between positions 53 and 54: VDS-QL.

TABLE 13D
Encoded NOV13b protein sequence.

(SEQ ID NO:58)

MRQINQTQVTEFLLLGLCDGPHTEQLLFIVLLGVYLVTVLGNLLLISLVHVDSQLHTPMYFFLCNLSLADLC
FSTNIVPQALIHLLSRKKVIAFTLCAARLLFFLIFGCTQCALLAVMSYDRYVAICNPLRYPNIMTWKVCVQL
ATGSWTSGILVSVVDTTFTLRLPYRGSNSIAHFFCEAPALLILASTDTHASEMAIFLMGVVILLIPVFLILV
SYGRIIVTVVKMKSTVGSLKAFSTCGSHLMVVILFYGSAIITCMTPKSSKQQEKSVSVFYAIVTPMLNPLIY
SLRNKDVKAALRKVATRNFP

A search of sequence databases reveals that the NOV13 amino acid sequence has 52 of 179 amino acid residues (29%) identical to, and 86 of 179 amino acid residues (48%) similar to, the 339 amino acid residue ptnr: SWISSPROT-ACC:Q13304 protein from Homo sapiens Putative G Protein-Coupled Receptor GPR17 (R12) (E=3.3e⁻¹⁵⁷).

NOV13b is predicted to be expressed in at least Apical microvilli of the retinal pigment epithelium, arterial (aortic), basal forebrain, brain, Burkitt lymphoma cell lines, corpus callosum, cardiac (atria and ventricle), caudate nucleus, CNS and peripheral tissue, cerebellum, cerebral cortex, colon, cortical neurogenic cells, endothelial (coronary artery and umbilical vein) cells, palate epithelia, eye, neonatal eye, frontal cortex, fetal hematopoietic cells, heart, hippocampus, hypothalamus, leukocytes, liver, fetal liver, lung, lung lymphoma cell lines, fetal lymphoid tissue, adult lymphoid tissue, Those that express MHC II and III nervous, medulla, subthalamic nucleus, ovary, pancreas, pituitary, placenta, pons, prostate, putamen, serum, skeletal muscle, small intestine, smooth muscle (coronary artery in aortic) spinal cord, spleen, stomach, taste receptor cells of the tongue, testis, thalamus, and thymus tissue. This information was derived by determining the tissue sources of the sequences that were included in the invention including but not limited to SeqCalling sources, Public EST sources, Literature sources, and/or RACE sources.

The disclosed NOV13a polypeptide has homology to the amino acid sequences shown in the BLASTP data listed in Table 13E.

TABLE 13E
BLAST results for NOV13a

| Gene Index/ | | Length | Identity | Positives | |
|---|---|---|---|---|---|
| Identifier | Protein/Organism | (aa) | (%) | (%) | Expect |
| gi\|14423807\|sp\|Q9H210\| | OLFACTORY | 308 | 307/308 | 308/308 | e−148 |
| O2D2_HUMAN | RECEPTOR 2D2 | | (99%) | (99%) | |
| | (OLFACTORY | | | | |
| | RECEPTOR 11-610) | | | | |
| | (OR11-610) (HB2) | | | | |
| gi\|17461460\|ref\|XP_{—} | similar to hB2 | 308 | 308/308 | 308/308 | e−148 |
| 062286.1\| | olfactory | | (100%) | (100%) | |
| (XM_062286) | receptor (H. | | | | |
| | sapiens) [Homo | | | | |
| | sapiens] | | | | |
| gi\|12007409\|gb\|AAG45183.1\| | B2 olfactory | 314 | 261/305 | 278/305 | e−127 |
| (AF321233) | receptor [Mus | | (85%) | (90%) | |
| | musculus] | | | | |
| gi\|3831619\|gb\|AAC70020.1\| | olfactory | 217 | 216/217 | 217/217 | e−100 |
| (AF065876) | receptor [Homo | | (99%) | (99%) | |
| | sapiens] | | | | |
| gi\|15293767\|gb\|AAK95076.1\| | olfactory | 214 | 213/214 | 214/214 | e−100 |
| (AF399591) | receptor [Homo | | (99%) | (99%) | |
| | sapiens] | | | | |

The homology between these and other sequences is shown graphically in the ClustalW analysis shown in Table 13F. In the ClustalW alignment of the NOV13 protein, as well as all other ClustalW analyses herein, the black outlined amino acid residues indicate regions of conserved sequence (i.e., regions that may be required to preserve structural or functional properties), whereas non-highlighted amino acid residues are less conserved and can potentially be altered to a much broader extent without altering protein structure or function.

[image]

[image]

Table 13G lists the domain description from DOMAIN analysis results against NOV13. This indicates that the NOV13 sequence has properties similar to those of other proteins known to contain this domain.

TABLE 13G
Domain Analysis of NOV13

gnl|Pfam|pfam00001, 7tm_1, 7 transmembrane receptor (rhodopsin

| | |
|---|---|
| family). | (SEQ ID NO:810) |

CD-Length = 254 residues, 94.9% aligned
Score = 93.2 bits (230), Expect = 2e−20

| | | | |
|---|---|---|---|
| NOV13: | 54 | QLHTPMYFFLCNLSLADLCFSTNIVPQALVHLLSRKKVIAFTLCAARLLFFLIFGCTQCA | 113 |
| | | +\| \|\| \|\| \|\|++\|\|\| \| + \| \|\| +\|+ \| \|\| \|++ \| | |
| Sbjct: | 14 | KLRTPTNIFLLNLAVADLLFLLTLPPWALYYLVGGDWVFGDALCKLVGALFVVNGYASIL | 73 |
| NOV13: | 114 | LLAVMSYDRYVAICNPLRYPNIMTWKVCVQLATGSWTSGILVSVVDTTFTLRLPYRGSNS | 173 |
| | | \|\| +\| \|\|\|+\|\| +\|\|\|\| \| \| + \| \| +\|+\|+ \|+ \|+ | |
| Sbjct: | 74 | LLTAISIDRYLAIVHPLRYRRIRTPRRAKVLILLVWVLALLLSLPPLLFSWLRTVEEGNT | 133 |
| NOV13: | 174 | IAHFFC-----EAPALLILASTDTHASEMAIFLTGVVILLIPVFLILVSYGRIIVTVVKM | 228 |
| | | + ++\|++ + + \| \| \|\|+ \|+ \| | |
| Sbjct: | 134 | TVCLIDFPEESVKRSYVLLSTLVGFVLPLLVILV--------------CYTRILRTLRKR | 179 |
| NOV13: | 229 | KSTVGSLK---------AFSTCGSHLMVVILFYGSAIITYMTPKSSKQQEKSVSVFYAI- | 278 |
| | | + \|\|\| \| ++ \|+ + \|+ + + + \| | |
| Sbjct: | 180 | ARSQRSLKRRSSSERKAAKMLLVVVVVFVLCWLPYHIVLLLDSLCLLSIWRVLPTALLIT | 239 |
| NOV13: | 279 | -----VTPMLNPLIY 288 | |
| | | \| \|\|\|+\|\| | |
| Sbjct: | 240 | LWLAYVNSCLNPIIY 254 | |

G-Protein Coupled Receptor (GPCRs) have been identified as extremely large subfamily of G protein-coupled receptors in a number of species. These receptors share a seven transmembrane domain structure with many neurotransmitter and hormone receptors, and are likely to underlie the recognition and G-protein-mediated transduction of various signals. Previously, GPCR genes cloned in different species were from random locations in the respective genomes. The human GPCR genes are intron less and belong to four different gene subfamilies, displaying great sequence variability. These genes are dominantly expressed in olfactory epithelium.

Olfactory receptors (ORs) have been identified as extremely large subfamily of G protein-coupled receptors in a number of species. These receptors share a seven transmembrane domain structure with many neurotransmitter and hormone receptors, and are likely to underlie the recognition and G-protein-mediated transduction of odorant signals. Previously, OR genes cloned in different species were from random locations in the respective genomes. The human OR genes are intron less and belong to four different gene subfamilies, displaying great sequence variability. These genes are dominantly expressed in olfactory epithelium.

The disclosed NOV13 nucleic acid of the invention encoding a G-Protein Coupled Receptor-like protein includes the nucleic acid whose sequence is provided in Table 13A, 14C or a fragment thereof. The invention also includes a mutant or variant nucleic acid any of whose bases may be changed from the corresponding base shown in Table 13A, or 14C while still encoding a protein that maintains its G-Protein Coupled Receptor-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 1 percent of the bases may be so changed.

The disclosed NOV13 protein of the invention includes the G-Protein Coupled Receptor-like protein whose sequence is provided in Table 13B, or 14D. The invention also includes a mutant or variant protein any of whose residues may be changed from the corresponding residue shown in Table 13B, or 14D while still encoding a protein that maintains its G-Protein Coupled Receptor-like activities and physiological functions, or a functional fragment thereof. In the mutant or variant protein, up to about 15 percent of the residues may be so changed.

The invention further encompasses antibodies and antibody fragments, such as F_{ab} or (F_{ab})₂, that bind immunospecifically to any of the proteins of the invention.

The above disclosed information suggests that this G-Protein Coupled Receptor-like protein (NOV13) is a member of a “G-Protein Coupled Receptor family”. Therefore, the NOV13 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV13 nucleic acids and proteins of the invention are useful in potential therapeutic applications implicated in developmental diseases, MHCII and III diseases (immune diseases), Taste and scent detectability Disorders, Burkitt's lymphoma, Corticoneurogenic disease, Signal Transduction pathway disorders, Retinal diseases including those involving photoreception, Cell Growth rate disorders; Cell Shape disorders, Feeding disorders;control of feeding; potential obesity due to over-eating; potential disorders due to starvation (lack of apetite), noninsulin-dependent diabetes mellitus (NIDDM1), bacterial, fungal, protozoal and viral infections (particularly infections caused by HIV-1 or HIV-2), pain, cancer (including but not limited to Neoplasm; adenocarcinoma; lymphoma; prostate cancer; uterus cancer), anorexia, bulimia, asthma, Parkinson's disease, acute heart failure, hypotension, hypertension, urinary retention, osteoporosis, Crohn's disease; multiple sclerosis; and Treatment of Albright Hereditary Ostoeodystrophy, angina pectoris, myocardial infarction, ulcers, asthma, allergies, benign prostatic hypertrophy, and psychotic and neurological disorders, including anxiety, schizophrenia, manic depression, delirium, dementia, severe mental retardation. Dentatorubro-pallidoluysian atrophy(DRPLA) Hypophosphatemic rickets, autosomal dominant (2) Acrocallosal syndrome and dyskinesias, such as Huntington's disease or Gilles de la Tourette syndrome, and/or other diseases and pathologies.

NOV13 nucleic acids and polypeptides are further useful in the generation of antibodies that bind immuno-specifically to the novel NOV13 substances for use in therapeutic or diagnostic methods. These antibodies may be generated according to methods known in the art, using prediction from hydrophobicity charts, as described in the “Anti-NOVX Antibodies” section below. The disclosed NOV13 protein has multiple hydrophilic regions, each of which can be used as an immunogen. These novel proteins can be used in assay systems for functional analysis of various human disorders, which will help in understanding of pathology of the disease and development of new drug targets for various disorders.

NOV14

A disclosed NOV14 nucleic acid of 986 nucleotides (also referred to as CG56023-01) encoding a novel G-Protein Coupled Receptor-like protein is shown in Table 14A. An open reading frame was identified beginning with an ATG initiation codon at nucleotides 23-25 and ending with a TGA codon at nucleotides 974-976. The start and stop codons are shown in bold in Table 14A, and the 5′ and 3′ untranslated regions, if any, are underlined.

TABLE 14A
NOV14 nucleotide sequence.

(SEQ ID NO:59)

CTGGGGATTTATGCCCATACTTATGGCTATAGGAAACTGGACAGAAATAAGTGAATTTATCCTCATGAGCTT
CTCTTCCCTACCTACTGAAATACAGTCATTGCTCTTCCTGACATTTCTAACTATCTATTTGGTTACTCTGAA
GGGAAACAGCCTCATCATTCTGGTTACCCTAGCTGACCCCATGCTACACAGCCCCATGTACTTCTTCCTCAG
AAACTTATCTTTCCTGGAGATTGGCTTCAACCTAGTCATTGTGCCCAAAATGCTGGGGACCCTGCTTGCCCA
GGACACAACCATCTCCTTCCTTGGCTGTGCCACTCAGATGTATTTCTTCTTCTTCTTTGGGGTAGCTGAATG
CTTCCTCCTGGCTACCATGGCATATGACCGCTATGTGGCCATCTGCAGTCCCTTGCACTACCCAGTCATCAT
GAACCAAAGGACACGGGCCAAACTGGCTGCTGCTTCCTGGTTCCCAGGCTTTCCTGTAGCTACTGTGCAGAC
CACATGGCTCTTCAGTTTTCCATTCTGTGGCACCAACAAGGTGAACCACTTCTTCTGTGACAGCCCGCCTGT
GCTGAAGCTGGTCTGTGCAGACACAGCACTGTTTGAGATCTACGCCATCGTCGGAACCATTCTGGTGGTCAT
GATCCCCTGCTTGCTGATCTTGTGTTCCTATACTCGCATTGCTGCTGCTATCCTCAAGATCCCATCAGCTAA
AGGGAAGCATAAAGCCTTCTCTACGTGCTCCTCACACCTCCTTGTTGTCTCTCTTTTCTATATATCTTCTAG
CCTCACCTACTTCTGGCCTAAATCAAATAATTCTCCTGAGAGCAAGAAGTTGTTATCATTATCCTACACTGT
TGTGACTCCCATGTTGAACCCCATTATCTACAGCTTGAGAAATAGCGAGGTGAAGAATGCCCTCAGCAGGAC
CTTCCACAAGGTCCTAGCCCTCAGAAACTGTATCCCATAGACCTTAGGAA

The disclosed NOV14 polypeptide (SEQ ID NO:60) encoded by SEQ ID NO:59 has 321 amino acid residues and is presented in Table 14B using the one-letter amino acid code.

TABLE 14B
Encoded NOV14 protein sequence.

| | |
|---|---|
| MPILMAIGNWTEISEFILMSFSSLPTEIQSLLFLTFLTIYLVTLKGNSLIILVTLADPMLHSPM | (SEQ ID NO:60) |
| YFFLRNLSFLEIGFNLVIVPKMLGTLLAQDTTISFLGCATQMYFFFFFGVAECFLLATMAYDRY | |
| VAICSPLHYPVIMNQRTRAKLAAASWFPGFPVATVQTTWLFSFPFCGTNKVNHFFCDSPPVLKL | |
| VCADTALFEIYAIVGTILVVMIPCLLILCSYTRIAAAILKIPSAKGKHKAFSTCSSHLLVVSLF | |
| YISSSLTYFWPKSNNSPESKKLLSLSYTVVTPMLNPIIYSLRNSEVKNALSRTFHKVLALRNCIP | |

A search of sequence databases reveals that the NOV14 amino acid sequence has 234 of 310 amino acid residues (75%) identical to, and 264 of 310 amino acid residues (85%) similar to, the 315 amino acid residue ptnr: SPTREMBL-ACC:Q9JKA6 protein from Mus musculus (Mouse) (OLFACTORY RECEPTOR P2) (E=4.0e⁻¹²⁴).

The disclosed NOV14 polypeptide has homology to the amino acid sequences shown in the BLASTP data listed in Table 14C.

TABLE 14C
BLAST results for NOV14

| Gene Index/ | | Length | Identity | Positives | |
|---|---|---|---|---|---|
| Identifier | Protein/Organism | (aa) | (%) | (%) | Expect |
| gi\|14423805\|sp\|Q9H207\| | OLFACTORY | 317 | 317/317 | 317/317 | e−154 |
| OAA5_HUMAN | RECEPTOR 10A5 | | (100%) | (100%) | |
| | (HP3) | | | | |
| gi\|12007437\|gb\|AAG45207.1\| | hP4 olfactory | 317 | 300/317 | 305/317 | e−145 |
| AF321237_4 | receptor [Homo | | (94%) | (95%) | |
| (AF321237) | sapiens] | | | | |
| gi\|12007412\|gb\|AAG45186.1\| | P3 olfactory | 317 | 292/316 | 302/316 | e−140 |
| (AF321233) | receptor [Mus | | (92%) | (95%) | |
| | musculus] | | | | |
| gi\|15419583\|gb\|AAK97076.1\| | olfactory | 324 | 294/320 | 304/320 | e−140 |
| AF293080_1 | receptor P3 [Mus | | (91%) | (94%) | |
| (AF293080) | musculus] | | | | |
| gi\|12007411\|gb\|AAG45185.1\| | P4 olfactory | 317 | 281/316 | 296/316 | e−136 |
| (AF321233) | receptor [Mus | | (88%) | (92%) | |
| | musculus] | | | | |

The homology between these and other sequences is shown graphically in the ClustalW analysis shown in Table 14F. In the ClustalW alignment of the NOV14 protein, as well as all other ClustalW analyses herein, the black outlined amino acid residues indicate regions of conserved sequence (i.e., regions that may be required to preserve structural or functional properties), whereas non-highlighted amino acid residues are less conserved and can potentially be altered to a much broader extent without altering protein structure or function.

[image]

[image]

Table 14E lists the domain descriptions from DOMAIN analysis results against NOV14. This indicates that the NOV14 sequence has properties similar to those of other proteins known to contain this domain.

TABLE 14E
Domain Analysis of NOV14

gnl|Pfam|pfam00001, 7tm_1, 7 transmembrane receptor (rhodopsin

| | |
|---|---|
| family). | (SEQ ID NO:810) |

CD-Length = 254 residues, 100.0% aligned
Score = 103 bits (256), Expect = 2e−23

| | | | |
|---|---|---|---|
| NOV14: | 46 | GNSLIILVTLADPMLHSPMYFFLRNLSFLEIGFNLVIVPKMLGTLLAQDTTISFLGCATQ | 105 |
| | | \|\| \|+\|\|\| \| \| +\| \|\| \|\|+ ++ \| \| + \| \| \|+ \| \| | |
| Sbjct: | 1 | GNLLVILVILRTKKLRTPTNIFLLNLAVADLLFLLTLPPWALYYLVGGDWVFGDALCKLV | 60 |
| NOV14: | 106 | MYFFFFFGVAECFLLATMAYDRYVAICSPLHYPVIMNQRTRAKLAAASWFPGFPVATVQT | 165 |
| | | \| \| \| \|\| ++ \|\|\|+\|\| \|\| \| \| \| \| \| | |
| Sbjct: | 61 | GALFVVNGYASILLLTAISIDRYLAIVHPLRYRRIRTPRRAKVLILLVWVLAL------- | 113 |
| NOV14: | 166 | TWLFSFPFCGTNKVNHFFCDSPPVLKLVCADTALFEIYAIVGTILVVMIPCLLILCSYTR | 225 |
| | | \| \| \| + + + \| + + ++ \| ++ \|++ ++\| \|+\|\| \|\|\| | |
| Sbjct: | 114 | --LLSLPPLLFSWLRTVEEGNTTVCLIDFPEESVKRSYVLLSTLVGFVLPLLVILVCYTR | 171 |
| NOV14: | 226 | IA---------AAILKIPSAKGKHKAFSTCSSHLLVVSLFY----ISSSLTYFWPKSNNS | 272 |
| | | \| \|\| \|+ + \| ++ \| + + + | |
| Sbjct: | 172 | ILRTLRKRARSQRSLKRRSSSERKAAKMLLVVVVVFVLCWLPYHIVLLLDSLCLLSIWRV | 231 |
| NOV14: | 273 | PESKKLLSLSYTVVTPMLNPIIY 295 | |
| | | + \|++\| \| \|\|\|\|\|\| | |
| Sbjct: | 232 | LPTALLITLWLAYVNSCLNPIIY 254 | |

G-Protein Coupled Receptor (GPCRs) have been identified as extremely large subfamily of G protein-coupled receptors in a number of species. These receptors share a seven transmembrane domain structure with many neurotransmitter and hormone receptors, and are likely to underlie the recognition and G-protein-mediated transduction of various signals. Previously, GPCR genes cloned in different species were from random locations in the respective genomes. The human GPCR genes are intron less and belong to four different gene subfamilies, displaying great sequence variability. These genes are dominantly expressed in olfactory epithelium.

Olfactory receptors (ORs) have been identified as extremely large subfamily of G protein-coupled receptors in a number of species. These receptors share a seven transmembrane domain structure with many neurotransmitter and hormone receptors, and are likely to underlie the recognition and G-protein-mediated transduction of odorant signals. Previously, OR genes cloned in different species were from random locations in the respective genomes. The human OR genes are intron less and belong to four different gene subfamilies, displaying great sequence variability. These genes are dominantly expressed in olfactory epithelium.

The disclosed NOV14 nucleic acid of the invention encoding a G-Protein Coupled Receptor-like protein includes the nucleic acid whose sequence is provided in Table 14A or a fragment thereof. The invention also includes a mutant or variant nucleic acid any of whose bases may be changed from the corresponding base shown in Table 14A while still encoding a protein that maintains its G-Protein Coupled Receptor-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

The disclosed NOV14 protein of the invention includes the G-Protein Coupled Receptor-like protein whose sequence is provided in Table 14B. The invention also includes a mutant or variant protein any of whose residues may be changed from the corresponding residue shown in Table 14B while still encoding a protein that maintains its G-Protein Coupled Receptor-like activities and physiological functions, or a functional fragment thereof. In the mutant or variant protein, up to about 12 percent of the residues may be so changed.

The invention further encompasses antibodies and antibody fragments, such as F_{ab} or (F_{ab})₂, that bind immunospecifically to any of the proteins of the invention.

The above disclosed information suggests that this G-Protein Coupled Receptor-like protein (NOV14) is a member of a “G-Protein Coupled Receptor family”. Therefore, the NOV14 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV14 nucleic acids and proteins of the invention are useful in potential therapeutic applications implicated in developmental diseases, MHCII and III diseases (immune diseases), Taste and scent detectability Disorders, Burkitt's lymphoma, Corticoneurogenic disease, Signal Transduction pathway disorders, Retinal diseases including those involving photoreception, Cell Growth rate disorders; Cell Shape disorders, Feeding disorders;control of feeding; potential obesity due to over-eating; potential disorders due to starvation (lack of apetite), noninsulin-dependent diabetes mellitus (NIDDM1), bacterial, fungal, protozoal and viral infections (particularly infections caused by HIV-1 or HIV-2), pain, cancer (including but not limited to Neoplasm; adenocarcinoma; lymphoma; prostate cancer; uterus cancer), anorexia, bulimia, asthma, Parkinson's disease, acute heart failure, hypotension, hypertension, urinary retention, osteoporosis, Crohn's disease; multiple sclerosis; and Treatment of Albright Hereditary Ostoeodystrophy, angina pectoris, myocardial infarction, ulcers, asthma, allergies, benign prostatic hypertrophy, and psychotic and neurological disorders, including anxiety, schizophrenia, manic depression, delirium, dementia, severe mental retardation. Dentatorubro-pallidoluysian atrophy(DRPLA) Hypophosphatemic rickets, autosomal dominant (2) Acrocallosal syndrome and dyskinesias, such as Huntington's disease or Gilles de la Tourette syndrome, and/or other diseases and pathologies.

NOV14 nucleic acids and polypeptides are further useful in the generation of antibodies that bind immuno-specifically to the novel NOV14 substances for use in therapeutic or diagnostic methods. These antibodies may be generated according to methods known in the art, using prediction from hydrophobicity charts, as described in the “Anti-NOVX Antibodies” section below. The disclosed NOV14 protein has multiple hydrophilic regions, each of which can be used as an immunogen. These novel proteins can be used in assay systems for functional analysis of various human disorders, which will help in understanding of pathology of the disease and development of new drug targets for various disorders.

NOV15

NOV15 includes three novel G-Protein Coupled Receptor-like proteins disclosed below. The disclosed sequences have been named NOV15a and NOV15b.

NOV15a

A disclosed NOV15a nucleic acid of 943 nucleotides (also referred to as CG56065-01) encoding a novel G-Protein Coupled Receptor-like protein is shown in Table 15A. An open reading frame was identified beginning with an ATG initiation codon at nucleotides 2-4 and ending with a TGA codon at nucleotides 935-937. The start and stop codons are shown in bold in Table 15A, and the 5′ and 3′ untranslated regions, if any, are underlined.

TABLE 15A
NOV15a nucleotide sequence.

| | |
|---|---|
| AATGGCAGCAGAAAACCATTCTTTTGTGACTAAGTTTATTCTGGTTGGGCTAACAGAGAAGTCAG | (SEQ ID NO:61) |
| AGCTACAGCTGCCCCTCTTCCTCGTCTTCCTGGGAATCTATGTAGTCACAGTCCTGGGGAACCTG | |
| GGCATGATCACACTGATTGGGCTCAGTTCTCACCTGCACACACCTATGTACTGTTTCCTCAGCAG | |
| TCTGTCCTTCATTGACTTCTGCCATTCCACTGTCATTACCCCTAAGATGCTGGTGAACTTTGTGA | |
| CAGAGAAGAACATCATCTCCTACCCTGAATGCATGACTCAGCTCTACTTCTTCCTCGTTTTTGCT | |
| ATTGCAGAGTGTCACATGTTGGCTGCAATGGCATATGACGGCTACGTGGCCATCTGTAGCCCCTT | |
| GCTGTACAGCATCATCATATCCAATAAGGCTTGCTTTTCTCTGATTTTAGTGGTGTATGTAATAG | |
| GCCTGATTTGTGCGTCAGCTCATATAGGCTGTATGTTTAGGGTTCAATTCTGCAAATTTGATGTG | |
| ATCAACCATTATTTCTGTGATCTTATTTCTATCTTGAAGCTCTCCTGTTCTAGTACTTACATTAA | |
| TGAGTTACTGATTTTAATCTTTAGTGGAATTAACATCCTTGTCCCCAGCCTGACCATCCTCAGCT | |
| CTTACATCTTCATCATTGCCAGCATCCTCCGCATTCGCTACACTGAGGGCAGGTCCAAAGCCTTC | |
| AGCACTTGCAGCTCCCACATCTCGGCTGTTTCTGTTTTCTTTGGGTCTGCAGCATTCATGTACCT | |
| GCAGCCATCATCTGTCAGCTCCATGGACCAGGGGAAAGTGTCCTCTGTGTTTTATACTATTGTTG | |
| TGCCCATGCTGAACCCCCTGATCTACAGCCTGAGGAATAAAGATGTCCACGTTGCCCTGAAGAAA | |
| ACGCTAGGGAAAAGAACATTCTTATGAACAGAA | |

The disclosed NOV15a polypeptide (SEQ ID NO:62) encoded by SEQ ID NO:61 has 311 amino acid residues and is presented in Table 15B using the one-letter amino acid code.

TABLE 15B
Encoded NOV15a protein sequence.

| | |
|---|---|
| MAAENHSFVTKFILVGLTEKSELQLPLFLVFLGIYVVTVLGNLGMITLIGLSSHLHTPMYCFLS | (SEQ ID NO:62) |
| SLSFIDFCHSTVITPKMLVNFVTEKNIISYPECMTQLYFFLVFAIAECHMLAAMAYDGYVAICS | |
| PLLYSIIISNKACFSLILVVYVIGLICASAHIGCMFRVQFCKFDVINHYFCDLISILKLSCSST | |
| YINELLILIFSGINILVPSLTILSSYIFIIASILRIRYTEGRSKAFSTCSSHISAVSVFFGSAA | |
| FMYLQPSSVSSMDQGKVSSVFYTIVVPMLNPLIYSLRNKDVHVALKKTLGKRTFL | |

A search of sequence databases reveals that the NOV15a amino acid sequence has 235 of 311 amino acid residues (75%) identical to, and 270 of 311 amino acid residues (86%) similar to, the 311 amino acid residue ptnr: SPTREMBL-ACC:O35184 protein from Rattus norvegicus (Rat) (Olfactory Receptor) (E=9.9e⁻¹²¹).

NOV15b

A disclosed NOV15b nucleic acid of 943 nucleotides (also referred to as CG56065-02) encoding a novel G-Protein Coupled Receptor-like protein is shown in Table 15C. An open reading frame was identified beginning with an ATG initiation codon at nucleotides 2-4 and ending with a TGA codon at nucleotides 935-937. The start and stop codons are shown in bold in Table 15C, and the 5′ and 3′ untranslated regions, if any, are underlined.

TABLE 15C
NOV15b nucleotide sequence.

(SEQ ID NO:63)

AATGGCAGCAGAAAACCATTCTTTTGTGACTAAGTTTATTCTGGTTGGGCTAACAGAGAAGTCAGAGCTACA
GCTGCCCCTCTTCCTCGTCTTCCTGGGAATCTATGTAGTCACAGTGCTGGGGAACCTGGGCATGATCACACT
GATTGGGCTCAGTTCTCACCTGCACACACCTATGTACTGTTTCCTCAGCAGTCTGTCCTTCATTGACTTCTG
CCATTCCACTGTCATTACCCCTAAGATGCTGGTGAACTTTGTGACAGAGAAGAACATCATCTCCTACCCTGA
ATGCATGACTCAGCTCTACTTCTTCCTCGTTTTTGCTATTGCAGAGTGTCACATGTTGGCTGCAATGGCATA
TGACGGCTACGTGGCCATCTGTAGCCCCGTGCTGTACAGCATCATCATATCCAATAAGGCTTGCTTTTCTCT
GATTTTAGTGGTGTATGTAATAGGCCTGATTTGTGCGTCAGCTCATATAGGCTGTATGTTTAGGGTTCAATT
CTGCAAATTTGATGTGATCAACCATTATTTCTGTGATCTTATTTCTATCTTGAAGCTCTCCTGTTCTAGTAC
TTACATTAATGAGTTACTGATTTTAATCTTTAGTGGAATTAACATCCTTGTCCCCAGCCTGACCATCCTCAG
CTCTTACATCTTCATCATTGCCAGCATCCTCCGCATTCGCTACACTGAGGGCAGGTCCAAAGCCTTCAGCAC
TTGCAGCTCCCACATCTCGGCTGTTTCTGTTTTCTTTGGGTCTGCAGCATTCATGTACCTGCAGCCATCATC
TGTCAGCTCCATGGACCAGGGGAAAGTGTCCTCTGTGTTTTATACTATTGTTGTGCCCGTGCTGAACCCCCT
GATCTACAGCCTGAGGAATAAAGATGTCCACGTTGCCCTGAAGAAAACGCTAGGGAAAAGAACATTCTTATG
AACAGAA

In a search of public sequence databases, the NOV15b nucleic acid sequence, localized to chromosome 4, has 770 of 937 bases (82%) identical to a gb:GENBANK-ID:AF282271|acc:AF282271.1 mRNA from Mus musculus (odorant receptor K11 gene, complete cds) (E=5.2e⁻¹³⁵).

The disclosed NOV15b polypeptide (SEQ ID NO:64) encoded by SEQ ID NO:63 has 311 amino acid residues and is presented in Table 15D using the one-letter amino acid code. Signal P, Psort and/or Hydropathy results predict that NOV15b has no signal peptide and is likely to be localized to the plasma membrane with a certainty of 0.6000. Alternatively, NOV15b may also localize to the Golgi body with a certainty of 0.4000, the endoplasmic reticulum (membrane) with a certainty of 0.3000, or in the microbody (peroxisome) with a certainty of 0.3000. The most likely cleavage site for NOV15b is between positions 41 and 42: VLG-NL.

TABLE 15D
Encoded NOV15b protein sequence.

(SEQ ID NO:64)

MAAENHSFVTKFILVGLTEKSELQLPLFLVFLGIYVVTVLGNLGMITLIGLSSHLHTPMYCFLSSLSFIDFC
HSTVITPKMLVNFVTEKNIISYPECMTQLYFFLVFAIAECHMLAAMAYDGYVAICSPVLYSIIISNKACFSL
ILVVYVIGLICASAHIGCMFRVQFCKFDVINHYFCDLISILKLSCSSTYINELLILIFSGINILVPSLTILS
SYIFIIASILRIRYTEGRSKAFSTCSSHISAVSVFFGSAAFMYLQPSSVSSMDQGKVSSVFYTIVVPVLNPL
IYSLRNKDVHVALKKTLGKRTFL

A search of sequence databases reveals that the NOV15b amino acid sequence has 237 of 311 amino acid residues (76%) identical to, and 273 of 311 amino acid residues (87%) similar to, the 314 amino acid residue ptnr:TREMBLNEW-ACC:AAG39856 protein from Mus musculus (Mouse) (Odorant Receptor K11) (E=2.6e⁻¹²⁵).

NOV15b is predicted to be expressed in at least the following tissues: Apical microvilli of the retinal pigment epithelium, arterial (aortic), basal forebrain, brain, Burkitt lymphoma cell lines, corpus callosum, cardiac (atria and ventricle), caudate nucleus, CNS and peripheral tissue, cerebellum, cerebral cortex, colon, cortical neurogenic cells, endothelial (coronary artery and umbilical vein) cells, palate epithelia, eye, neonatal eye, frontal cortex, fetal hematopoietic cells, heart, hippocampus, hypothalamus, leukocytes, liver, fetal liver, lung, lung lymphoma cell lines, fetal lymphoid tissue, adult lymphoid tissue, Those that express MHC II and III nervous, medulla, subthalamic nucleus, ovary, pancreas, pituitary, placenta, pons, prostate, putamen, serum, skeletal muscle, small intestine, smooth muscle (coronary artery in aortic) spinal cord, spleen, stomach, taste receptor cells of the tongue, testis, thalamus, and thymus tissue. This information was derived by determining the tissue sources of the sequences that were included in the invention including but not limited to SeqCalling sources, Public EST sources, Literature sources, and/or RACE sources.

The disclosed NOV15a polypeptide has homology to the amino acid sequences shown in the BLASTP data listed in Table 15E.

TABLE 15E
BLAST results for NOV15a

| Gene Index/ | | Length | Identity | Positives | |
|---|---|---|---|---|---|
| Identifier | Protein/Organism | (aa) | (%) | (%) | Expect |
| gi\|17472672\|ref\|XP_{—} | similar to | 311 | 311/311 | 311/311 | e−140 |
| 061794.1\| | odorant receptor | | (100%) | (100%) | |
| (XM_061794) | K11 (H. sapiens) | | | | |
| | [Homo sapiens] | | | | |
| gi\|11692519\|gb\|AAG39856.1\| | odorant receptor | 314 | 239/311 | 273/311 | e−110 |
| AF282271_1 | K11 [Mus | | (76%) | (86%) | |
| (AF282271) | musculus] | | | | |
| gi\|11692527\|gb\|AAG39860.1\| | odorant receptor | 311 | 236/311 | 271/311 | e−108 |
| AF282275_1 | K15 [Mus | | (75%) | (86%) | |
| (AF282275) | musculus] | | | | |
| gi\|17472662\|ref\|XP_{—} | similar to | 593 | 233/301 | 261/301 | e−105 |
| 061790.1\| | odorant receptor | | (77%) | (86%) | |
| (XM_061790) | K4h11 (H. | | | | |
| | sapiens) [Homo | | | | |
| | sapiens] | | | | |
| gi\|2317704\|gb\|AAB66333.1\| | olfactory | 311 | 235/311 | 270/311 | e−105 |
| (AF010293) | receptor [Rattus | | (75%) | (86%) | |
| | norvegicus] | | | | |

The homology between these and other sequences is shown graphically in the ClustalW analysis shown in Table 15F. In the ClustalW alignment of the NOV15 protein, as well as all other ClustalW analyses herein, the black outlined amino acid residues indicate regions of conserved sequence (i.e., regions that may be required to preserve structural or functional properties), whereas non-highlighted amino acid residues are less conserved and can potentially be altered to a much broader extent without altering protein structure or function.

[image]

[image]

[image]

Table 15G lists the domain description from DOMAIN analysis results against NOV15. This indicates that the NOV15 sequence has properties similar to those of other proteins known to contain this domain.

TABLE 15G
Domain Analysis of NOV15

gnl|Pfam|pfam00001, 7tm_1, 7 transmembrane receptor (rhodopsin
family). (SEQ ID NO:810)
CD-Length = 254 residues, 100.0% aligned
Score = 86.7 bits (213), Expect = 2e−18

| | | | |
|---|---|---|---|
| NOV15: | 41 | GNLGMITLIGLSSHLHTPMYCFLSSLSFIDFCHSTVITPKMLVNFVTEKNIISYPECMTQ | 100 |
| | | \|\|\| +\| +\| + \| \|\| \|\| +\|+ \| + \| \| \| + \| | |
| Sbjct: | 1 | GNLLVILVILRTKKLRTPTNIFLLNLAVADLLFLLTLPPWALYYLVGGDWVFGDALCKLV | 60 |
| NOV15: | 101 | LYFFLVFAIAECHMLAAMAYDGYVAICSPLLYSIIISNKACFSLILVVYVIGLICASAHI | 160 |
| | | \|+\| \| +\| \|++ \| \|+\|\| \|\| \| \| + + \|\|\|+\|+\|+ \|+ + + | |
| Sbjct: | 61 | GALFVVNGYASILLLTAISIDRYLAIVHPLRYRRIRTPRRAKVLILLVWVLALLLSLPPL | 120 |
| NOV15: | 161 | GCMFRVQFCKFDVINHYFCD-----LISILKLSCSSTYINELLILIFSGINILVPSLTIL | 215 |
| | | + + + \| + \|\| ++ \|\|+++ \|\| | |
| Sbjct: | 121 | LFSWLRTVEEGNTTVCLIDFPEESVKRSYVLLSTLVGFVLPLLVILVCYTRILRTLRKRA | 180 |
| NOV15: | 216 | SSYIFIIASILRIRYTEGRSKAFSTCSSHISAVSVFFGSAAFMYL----QPSSVSSMDQG | 271 |
| | | \| \|+ \| + \| \| + \| + + \| \| + | |
| Sbjct: | 181 | RSQ-----RSLKRRSSSERKAAKMLLVVVVVFVLCWLPYHIVLLLDSLCLLSIWRVLPTA | 235 |
| NOV15: | 272 | KVSSVFYTIVVPMLNPLIY 290 | |
| | | + +++ \| \|\|\|+\|\| | |
| Sbjct: | 236 | LLITLWLAYVNSCLNPIIY 254 | |

G-Protein Coupled Receptor (GPCRs) have been identified as extremely large subfamily of G protein-coupled receptors in a number of species. These receptors share a seven transmembrane domain structure with many neurotransmitter and hormone receptors, and are likely to underlie the recognition and G-protein-mediated transduction of various signals. Previously, GPCR genes cloned in different species were from random locations in the respective genomes. The human GPCR genes are intron less and belong to four different gene subfamilies, displaying great sequence variability. These genes are dominantly expressed in olfactory epithelium.

Olfactory receptors (ORs) have been identified as extremely large subfamily of G protein-coupled receptors in a number of species. These receptors share a seven transmembrane domain structure with many neurotransmitter and hormone receptors, and are likely to underlie the recognition and G-protein-mediated transduction of odorant signals. Previously, OR genes cloned in different species were from random locations in the respective genomes. The human OR genes are intron less and belong to four different gene subfamilies, displaying great sequence variability. These genes are dominantly expressed in olfactory epithelium.

The disclosed NOV15 nucleic acid of the invention encoding a G-Protein Coupled Receptor-like protein includes the nucleic acid whose sequence is provided in Table 15A, 15C or a fragment thereof. The invention also includes a mutant or variant nucleic acid any of whose bases may be changed from the corresponding base shown in Table 15A or 15C while still encoding a protein that maintains its G-Protein Coupled Receptor-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 18 percent of the bases may be so changed.

The disclosed NOV15 protein of the invention includes the G-Protein Coupled Receptor-like protein whose sequence is provided in Table 15B, or 15D. The invention also includes a mutant or variant protein any of whose residues may be changed from the corresponding residue shown in Table 15B, or 15D while still encoding a protein that maintains its G-Protein Coupled Receptor-like activities and physiological functions, or a functional fragment thereof. In the mutant or variant protein, up to about 23 percent of the residues may be so changed.

The invention further encompasses antibodies and antibody fragments, such as F_{ab} or (F_{ab})₂, that bind immunospecifically to any of the proteins of the invention.

The above disclosed information suggests that this G-Protein Coupled Receptor-like protein (NOV15) is a member of a “G-Protein Coupled Receptor family”. Therefore, the NOV15 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV15 nucleic acids and proteins of the invention are useful in potential therapeutic applications implicated in developmental diseases, MHCII and III diseases (immune diseases), Taste and scent detectability Disorders, Burkitt's lymphoma, Corticoneurogenic disease, Signal Transduction pathway disorders, Retinal diseases including those involving photoreception, Cell Growth rate disorders; Cell Shape disorders, Feeding disorders;control of feeding; potential obesity due to over-eating; potential disorders due to starvation (lack of apetite), noninsulin-dependent diabetes mellitus (NIDDM1), bacterial, fungal, protozoal and viral infections (particularly infections caused by HIV-1 or HIV-2), pain, cancer (including but not limited to Neoplasm; adenocarcinoma; lymphoma; prostate cancer; uterus cancer), anorexia, bulimia, asthma, Parkinson's disease, acute heart failure, hypotension, hypertension, urinary retention, osteoporosis, Crohn's disease; multiple sclerosis; and Treatment of Albright Hereditary Ostoeodystrophy, angina pectoris, myocardial infarction, ulcers, asthma, allergies, benign prostatic hypertrophy, and psychotic and neurological disorders, including anxiety, schizophrenia, manic depression, delirium, dementia, severe mental retardation. Dentatorubro-pallidoluysian atrophy(DRPLA) Hypophosphatemic rickets, autosomal dominant (2) Acrocallosal syndrome and dyskinesias, such as Huntington's disease or Gilles de la Tourette syndrome, and/or other diseases and pathologies.

NOV15 nucleic acids and polypeptides are further useful in the generation of antibodies that bind immuno-specifically to the novel NOV15 substances for use in therapeutic or diagnostic methods. These antibodies may be generated according to methods known in the art, using prediction from hydrophobicity charts, as described in the “Anti-NOVX Antibodies” section below. The disclosed NOV15 protein has multiple hydrophilic regions, each of which can be used as an immunogen. These novel proteins can be used in assay systems for functional analysis of various human disorders, which will help in understanding of pathology of the disease and development of new drug targets for various disorders.

NOV16a

A disclosed NOV16a nucleic acid of 891 nucleotides (also referred to as CG56067-01) encoding a novel G-Protein Coupled Receptor-like protein is shown in Table 16A. An open reading frame was identified beginning with an ATG initiation codon at nucleotides 5-7 and ending with a TAA codon at nucleotides 878-880. The start and stop codons are shown in bold in Table 16a, and the 5′ and 3′ untranslated regions, if any, are underlined.

TABLE 16A
NOV16a nucleotide sequence.

(SEQ ID NO:65)

GAAAATGTCAGCAGGAAACCATTCCTCAGTGACTGAGTTCATTCTGGCTGGGCTCTCAGAACAGCCAGAGCT
CCAGCTGCGCCTCTTCCTCCTGTTCTTAGGAATCTATGTGGTCACAGTGGTGGGCAACTTGAGCATGATCAC
ACTGATTGGGCTCAGTTCTCACCTGCATACCCCCATGTACTATTTCCTCAGTGGTCTGTCCTTCATTGATAT
CTGCCATTCCACTATCATTACCCCCAAAATGCTGGTGAACTTTGTGACAGAGAAGAACATCATCTCCTACCC
TGAATGCATGACTCAGCTTTACTTCTTCCTCATTTTTGCTATTGCAGAGTGTCACATGTTGGCTGTAACGGC
ATATGACCGCTATGTTGCCATCTGCAGCCCCTTGCTGTACAATGTCATCATGTCCTATCACCACTGCTTCTG
GCTCACAGTGGGAGTTTACATTTTAGGCATCCTTGGATCTACAATTCACACCGGCTTTATGTTGAGACTCTT
TTTGTGCAAGACTAATGTGATTAACCATTATTTTTGTGATCTCTTCCCTCTCTTGGGGCTCTCCTGCTCCAG
CACCTACATCAATGAATTACTGGTTCTGGTCTTGAGTGCATTTAACATCCTGACGCCTGCCTTAACCATCCT
TGCTTCTTACATCTTTATCATTGCCAGCATCCTCCGCATTCGCTCCACTGAGGGCAGGTCCAAAGCCTTCAG
CACTTGCAGCTCCCACATCTTGGCTGTTGCTGTTTTCTTTGGGTCTGCAGCATTCATGTACCTCCAGCCATC
ATCTGTCAGCTCCATGGACCAGGGGAAAGTGTCCTCTGTGTTTTATACTATTGTTGTGCCCATGCTGAACCC
CCAATCTATAGCCTAAGAAATAAGGAT

In a search of public sequence databases, the NOV16a nucleic acid sequence, localized to chromosome 4, has729 of 888 bases (82%) identical to a gb:GENBANK-ID:AF282293|acc:AF282293.1 mRNA from Mus musculus (odorant receptor K4h11 gene, complete cds) (E=9.8e⁻¹²⁷).

The disclosed NOV16a polypeptide (SEQ ID NO:66) encoded by SEQ ID NO:65 has 311 amino acid residues and is presented in Table 16B using the one-letter amino acid code. Signal P, Psort and/or Hydropathy results predict that NOV16a has no signal peptide and is likely to be localized to the plasma membrane with a certainty of 0.6000. Alternatively, NOV16A may also localize to the Golgi body with a certainty of 0.4000, the endoplasmic reticulum (membrane) with a certainty of 0.3000, or in the microbody (peroxisome) with a certainty of 0.3000. The most likely cleavage site for NOV16A is between positions 41 and 42: VVG-NL.

TABLE 16B
Encoded NOV16a protein sequence.

| | |
|---|---|
| MSAGNHSSVTEFILAGLSEQPELQLRLFLLFLGIYVVTVVGNLSMITLIGLSSHLHTPMYYFLS | (SEQ ID NO:66) |
| GLSFIDICHSTIITPKMLVNFVTEKNIISYPECMTQLYFFLIFAIAECHMLAVTAYDRYVAICS | |
| PLLYNVIMSYHHCFWLTVGVYILGILGSTIHTGFMLRLFLCKTNVINHYFCDLFPLLGLSCSST | |
| YINELLVLVLSAFNILTPALTILASYIFIIASILRIRSTEGRSKAFSTCSSHILAVAVFFGSAA | |
| FMYLQPSSVSSMDQGKVSSVFYTIVVPMLNPQSIA | |

A search of sequence databases reveals that the NOV16a amino acid sequence has 232 of 287 amino acid residues (80%) identical to, and 253 of 287 amino acid residues (88%) similar to, the 307 amino acid residue ptnr:TREMBLNEW-ACC:AAG39878 protein from Mus musculus (Mouse) (Odorant Receptor K4H11) (E=5.1e⁻¹²²).

NOV16a is predicted to be expressed in at least Apical microvilli of the retinal pigment epithelium, arterial (aortic), basal forebrain, brain, Burkitt lymphoma cell lines, corpus callosum, cardiac (atria and ventricle), caudate nucleus, CNS and peripheral tissue, cerebellum, cerebral cortex, colon, cortical neurogenic cells, endothelial (coronary artery and umbilical vein) cells, palate epithelia, eye, neonatal eye, frontal cortex, fetal hematopoietic cells, heart, hippocampus, hypothalamus, leukocytes, liver, fetal liver, lung, lung lymphoma cell lines, fetal lymphoid tissue, adult lymphoid tissue, Those that express MHC II and III nervous, medulla, subthalamic nucleus, ovary, pancreas, pituitary, placenta, pons, prostate, putamen, serum, skeletal muscle, small intestine, smooth muscle (coronary artery in aortic) spinal cord, spleen, stomach, taste receptor cells of the tongue, testis, thalamus, and thymus tissue. This information was derived by determining the tissue sources of the sequences that were included in the invention including but not limited to SeqCalling sources, Public EST sources, Literature sources, and/or RACE sources.

NOV16b

A disclosed NOV16b nucleic acid of 939 nucleotides (also referred to as CG56753-02) encoding a novel G-Protein Coupled Receptor-like protein is shown in Table 16C. An open reading frame was identified beginning with an ATG initiation codon at nucleotides 1-3 and ending with a TAG codon at nucleotides 934-936. The start and stop codons are shown in bold in Table 16C, and the 5′ and 3′ untranslated regions, if any, are underlined.

TABLE 16C
NOV16b nucleotide sequence.

(SEQ ID NO:67)

ATGTCAGGAGAAAATAATTCCTCAGTGACTGAGTTCATTCTGGCTGGGCTCTCAGAACAGCCAGAGCTCCAG
CTGCCCCTCTTCCTCCTGTTCTTAGGAATCTATGTGGTCACAGTGGTGGGCAACCTGGGCATGACCACACTG
ATTTGGCTCAGTTCTCACCTGCACACCCCTATGTACTATTTCCTCAGCAGTCTGTCCTTCATTGACTTCTGC
CATTCCACTGTCATTACCCCTAAGATGCTGGTGAACTTTGTGACAGAGAAGAACATCATCTCCTACCCTGAA
TCCATGACTCAGCTCTACTTCTTCCTCGTTTTTGCTATTGCAGAGTGTCACATGTTGGCTGCAATGGCGTAT
GACCGTTACATGGCCATCTGTAGCCCCTTGCTGTACAGTGTCATCATATCCAATAAGGCTTGCTTTTCTCTG
ATTTTAGGGGTGTATATAATAGGCCTGGTTTGTGCATCAGTTCATACAGACAGTATGTTTAGGGTTCAATTC
TGCAAATTTGATTTGATTAACCATTATTTCTGTGATCTTCTTCCCCTCCTAAAGCTCTCTTGCTCTAGTATC
TATGTCAACAAACTACTTATTCTATGTGTTGGTGCATTTAACATCCTTGTCCCCAGCCTGACCATCCTTTGC
TCTTACATCTTTATTATTGCCAGCATCCTCCACATTCGCTCCACTGAGGGCAGGTCCAAAGCCTTCAGCACT
TGTAGCTCCCACATGTTGGCGGTTGTAATCTTTTTTGGATCTGCAGCATTCATGTACTTGCAGCCATCTTCA
ATCAGCTCCATGGACCAGGGGAAAGTATCCTCTCTGTTTTATACTATTATTGTGCCCATGTTGAACCCTCTG
ATTTATAGCCTGAGGAATAAAGATGTCCATGTTTCCCTGAAGAAAATGCTACAGAGAAGAACATTATTGTAA
ACA

In a search of public sequence databases, the NOV16b nucleic acid sequence has 770 of 935 bases (82%) identical to a gb:GENBANK-ID:AF282271|acc:AF282271.1 mRNA from Mus musculus (odorant receptor K11 gene, complete cds) (E=1.3e⁻¹³⁶).

The disclosed NOV16b polypeptide (SEQ ID NO:68) encoded by SEQ ID NO:67 has 311 amino acid residues and is presented in Table 16D using the one-letter amino acid code. Signal P, Psort and/or Hydropathy results predict that NOV16b has A signal peptide and is likely to be localized to the plasma membrane with a certainty of 0.6000. Alternatively, NOV16b may also localize to the Golgi body with a certainty of 0.4000, the endoplasmic reticulum (membrane) with a certainty of 0.3000, or in the endoplasmic reticulum (lumen) with a certainty of 0.3000. The most likely cleavage site for NOV16b is between positions 41 and 42: VVG-NL.

TABLE 16D
Encoded NOV16b protein sequence.

(SEQ ID NO:68)

MSGENNSSVTEFILAGLSEQPELQLPLFLLFLGIYVVTVVGNLGMTTLIWLSSHLHTPMYYFLSSLSFIDFC
HSTVITPKNLVNFVTEKNIISYPECMTQLYFFLVFAIAECHMLAAMAYDRYMAICSPLLYSVIISNKACFSL
ILGVYIIGLVCASVHTDSMFRVQFCKFDLINHYFCDLLPLLKLSCSSIYVNKLLILCVGAFNILVPSLTILC
SYIFIIASILHIRSTEGRSKAFSTCSSHMLAVVIFFGSAAFMYLQPSSISSMDQGKVSSVFYTIIVPMLNPL
IYSLRNKDVHVSLKKMLQRRTLL

A search of sequence databases reveals that the NOV16b amino acid sequence has 238 of 311 amino acid residues (76%) identical to, and 274 of 311 amino acid residues (88%) similar to, the 314 amino acid residue ptnr:SPTREMBL-ACC:Q9EQB8 protein from Mus musculus (Mouse) (Odorant Receptor K11) (E=1.0e⁻¹²⁷).

NOV16b is predicted to be expressed in at least the following tissues: Apical microvilli of the retinal pigment epithelium, arterial (aortic), basal forebrain, brain, Burkitt lymphoma cell lines, corpus callosum, cardiac (atria and ventricle), caudate nucleus, CNS and peripheral tissue, cerebellum, cerebral cortex, colon, cortical neurogenic cells, endothelial (coronary artery and umbilical vein) cells, palate epithelia, eye, neonatal eye, frontal cortex, fetal hematopoietic cells, heart, hippocampus, hypothalamus, leukocytes, liver, fetal liver, lung, lung lymphoma cell lines, fetal lymphoid tissue, adult lymphoid tissue, Those that express MHC II and III nervous, medulla, subthalamic nucleus, ovary, pancreas, pituitary, placenta, pons, prostate, putamen, serum, skeletal muscle, small intestine, smooth muscle (coronary artery in aortic) spinal cord, spleen, stomach, taste receptor cells of the tongue, testis, thalamus, and thymus tissue. This information was derived by determining the tissue sources of the sequences that were included in the invention including but not limited to SeqCalling sources, Public EST sources, Literature sources, and/or RACE sources.

The disclosed NOV16a polypeptide has homology to the amino acid sequences shown in the BLASTP data listed in Table 16E.

TABLE 16E
BLAST results for NOV16a

| Gene Index/ | | Length | Identity | Positives | |
|---|---|---|---|---|---|
| Identifier | Protein/Organism | (aa) | (%) | (%) | Expect |
| gi\|17472662\|ref\|XP_{—} | similar to | 593 | 265/284 | 267/284 | e−121 |
| 061790.1\| | odorant receptor | | (93%) | (93%) | |
| (XM_061790) | K4h11 (H. | | | | |
| | sapiens) [Homo | | | | |
| | sapiens] | | | | |
| gi\|11692519\|gb\|AAG39856.1\| | odorant receptor | 314 | 223/287 | 250/287 | e−104 |
| AF282271_1 | K11 [Mus | | (77%) | (86%) | |
| (AF282271) | musculus] | | | | |
| gi\|11692563\|gb\|AAG39878.1\| | odorant receptor | 307 | 232/287 | 253/287 | e−102 |
| AF282293_1 | K4h11 [Mus | | (80%) | (87%) | |
| (AF282293) | musculus] | | | | |
| gi\|17472672\|ref\|XP_{—} | similar to | 311 | 226/287 | 252/287 | e−102 |
| 061794.1\| | odorant receptor | | (78%) | (87%) | |
| (XM_061794) | K11 (H. sapiens) | | | | |
| | [Homo sapiens] | | | | |
| gi\|11692527\|gb\|AAG39860.1\| | odorant receptor | 311 | 224/287 | 246/287 | e−102 |
| AF282275_1 | K15 [Mus | | (78%) | (85%) | |
| (AF282275) | musculus] | | | | |

The homology between these and other sequences is shown graphically in the ClustalW analysis shown in Table 16F. In the ClustalW alignment of the NOV16 protein, as well as all other ClustalW analyses herein, the black outlined amino acid residues indicate regions of conserved sequence (i.e., regions that may be required to preserve structural or functional properties), whereas non-highlighted amino acid residues are less conserved and can potentially be altered to a much broader extent without altering protein structure or function.

[image]

[image]

Table 16G lists the domain description from DOMAIN analysis results against NOV16. This indicates that the NOV16 sequence has properties similar to those of other proteins known to contain this domain.

TABLE 16G
Domain Analysis of NOV16

gnl|Pfam|pfam00001, 7tm_1, 7 transmembrane receptor (rhodopsin

| | |
|---|---|
| family). | (SEQ ID NO:810) |

CD-Length = 254 residues, 98.8% aligned
Score = 85.9 bits (211), Expect = 3e−18

| | | | |
|---|---|---|---|
| NOV18: | 41 | GNLSMITLIGLSSHLHTPMYYFLSGLSFIDICHSTIITPKMLVNFVTEKNIISYPECMTQ | 100 |
| | | \|\|\| +\| +\| + \| \|\| \|\| \|+ \|+ + \| \| \| + \| | |
| Sbjct: | 1 | GNLLVILVILRTKKLRTPTNIFLLNLAVADLLFLLTLPPWALYYLVGGDWVFGDALCKLV | 60 |
| NOV18: | 101 | LYFFLIFAIAECHMLAVTAYDRYVAICSPLLYNVIMSYHHCFWLTVGVYILGILGSTIHT | 160 |
| | | \|++ \| +\| + \|\|\|+\|\| \|\| \| \| + \| + \|++\| +\| \| | |
| Sbjct: | 61 | GALFVVNGYASILLLTAISIDRYLAIVHPLRYRRIRTPRRAKVLILLVWVLALLLSLPPL | 120 |
| NOV18: | 161 | GFMLRLFLCKTNVINHYFCDLFPLLG-----LSCSSTYINELLVLVLSAFNILTPALTIL | 215 |
| | | \| + + \| + \|\| ++ \|\|\|+++ \|\| | |
| Sbjct: | 121 | LFSWLRTVEEGNTTVCLIDFPEESVKRSYVLLSTLVGFVLPLLVILVCYTRIL-----RT | 175 |
| NOV18: | 216 | ASYIFIIASILRIRSTEGRSKAFSTCSSHILAVAVFFGSAAFMYL----QPSSVSSMDQG | 271 |
| | | \|+ \|\|+ \| \| ++ \| + + \| \| + | |
| Sbjct: | 176 | LRKRARSQRSLKRRSSSERKAAKMLLVVVVVFVLCWLPYHIVLLLDSLCLLSIWRVLPTA | 235 |
| NOV18: | 272 | KVSSVFYTIVVPMLNP 287 | |
| | | + +++ \| \|\|\| | |
| Sbjct: | 236 | LLITLWLAYVNSCLNP 251 | |

G-Protein Coupled Receptor (GPCRs) have been identified as extremely large subfamily of G protein-coupled receptors in a number of species. These receptors share a seven transmembrane domain structure with many neurotransmitter and hormone receptors, and are likely to underlie the recognition and G-protein-mediated transduction of various signals. Previously, GPCR genes cloned in different species were from random locations in the respective genomes. The human GPCR genes are intron less and belong to four different gene subfamilies, displaying great sequence variability. These genes are dominantly expressed in olfactory epithelium.

Olfactory receptors (ORs) have been identified as extremely large subfamily of G protein-coupled receptors in a number of species. These receptors share a seven transmembrane domain structure with many neurotransmitter and hormone receptors, and are likely to underlie the recognition and G-protein-mediated transduction of odorant signals. Previously, OR genes cloned in different species were from random locations in the respective genomes. The human OR genes are intron less and belong to four different gene subfamilies, displaying great sequence variability. These genes are dominantly expressed in olfactory epithelium.

The disclosed NOV16 nucleic acid of the invention encoding a G-Protein Coupled Receptor-like protein includes the nucleic acid whose sequence is provided in Table 16A or a fragment thereof. The invention also includes a mutant or variant nucleic acid any of whose bases may be changed from the corresponding base shown in Table 16A while still encoding a protein that maintains its G-Protein Coupled Receptor-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 18 percent of the bases may be so changed.

The disclosed NOV16 protein of the invention includes the G-Protein Coupled Receptor-like protein whose sequence is provided in Table 16B. The invention also includes a mutant or variant protein any of whose residues may be changed from the corresponding residue shown in Table 16B while still encoding a protein that maintains its G-Protein Coupled Receptor-like activities and physiological functions, or a functional fragment thereof. In the mutant or variant protein, up to about 23 percent of the residues may be so changed.

The invention further encompasses antibodies and antibody fragments, such as F_{ab} or (F_{ab})₂, that bind immunospecifically to any of the proteins of the invention.

The above disclosed information suggests that this G-Protein Coupled Receptor-like protein (NOV16) is a member of a “G-Protein Coupled Receptor family”. Therefore, the NOV16 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV16 nucleic acids and proteins of the invention are useful in potential therapeutic applications implicated in developmental diseases, MHCII and III diseases (immune diseases), Taste and scent detectability Disorders, Burkitt's lymphoma, Corticoneurogenic disease, Signal Transduction pathway disorders, Retinal diseases including those involving photoreception, Cell Growth rate disorders; Cell Shape disorders, Feeding disorders;control of feeding; potential obesity due to over-eating; potential disorders due to starvation (lack of apetite), noninsulin-dependent diabetes mellitus (NIDDM1), bacterial, fungal, protozoal and viral infections (particularly infections caused by HIV-1 or HIV-2), pain, cancer (including but not limited to Neoplasm; adenocarcinoma; lymphoma; prostate cancer; uterus cancer), anorexia, bulimia, asthma, Parkinson's disease, acute heart failure, hypotension, hypertension, urinary retention, osteoporosis, Crohn's disease; multiple sclerosis; and Treatment of Albright Hereditary Ostoeodystrophy, angina pectoris, myocardial infarction, ulcers, asthma, allergies, benign prostatic hypertrophy, and psychotic and neurological disorders, including anxiety, schizophrenia, manic depression, delirium, dementia, severe mental retardation. Dentatorubro-pallidoluysian atrophy(DRPLA) Hypophosphatemic rickets, autosomal dominant (2) Acrocallosal syndrome and dyskinesias, such as Huntington's disease or Gilles de la Tourette syndrome, and/or other diseases and pathologies.

NOV16 nucleic acids and polypeptides are further useful in the generation of antibodies that bind immuno-specifically to the novel NOV16 substances for use in therapeutic or diagnostic methods. These antibodies may be generated according to methods known in the art, using prediction from hydrophobicity charts, as described in the “Anti-NOVX Antibodies” section below. The disclosed NOV16 protein has multiple hydrophilic regions, each of which can be used as an immunogen. These novel proteins can be used in assay systems for functional analysis of various human disorders, which will help in understanding of pathology of the disease and development of new drug targets for various disorders.

NOV17

NOV17 includes three novel G-Protein Coupled Receptor-like proteins disclosed below. The disclosed sequences have been named NOV17a, NOV17b, NOV17c, and NOV17d.

NOV17a

A disclosed NOV17a nucleic acid of 962 nucleotides (also referred to as CG56657-01) encoding a novel G-Protein Coupled Receptor-like protein is shown in Table 17A. An open reading frame was identified beginning with an ATG initiation codon at nucleotides 18-20 and ending with a TAG codon at nucleotides 954-956. The start and stop codons are shown in bold in Table 17A, and the 5′ and 3′ untranslated regions, if any, are underlined.

TABLE 17A
NOV17a nucleotide sequence.

(SEQ ID NO:69)

GATCGTATGAATGCCCCATGGAAAATTACAATCAAACGTCAACTGATTTCATCTTATTGGGGCTGTTCCCAC
CATCAAAAATTGGCCTTTTCCTCTTCATTCTCTTTGTTCTCATTTTCCTAATGGCTCTAATTGGAAACCTAT
CCATGATTCTTCTCATCTTCTTGGACACCCATCTCCACACACCCATGTATTTCCTGCTTAGTCAGCTCTCCC
TCATTGACCTAAATTACATCTCTACGATTGTTCCTAAGATGGCTTCTGATTTTCTGTATGGAAACAAGTCTA
TCTCCTTCATTGGGTGTGGGATTCAGAGTTTCTTCTTCATGACTTTTGCAGGTGCAGAAGCGCTGCTCCTGA
CATCAATGGCCTATGATCGTTATGTGGCCATTTGCTTTCCTCTCCACTATCCCATCCGTATGAGCAAAAGAA
TGTATGTGCTGATGATAACAGGATCTTGGATGATAGGCTCCATCAACTCTTGTGCTCACACAGTATATGCAT
TCCGTATCCCATATTGCAAGTCCAGAGCCATCAATCATTTTTTCTGTGATGTTCCAGCTATGTTGACATTAG
CCTGTACAGACACCTGGCTCTATGAGTACACAGTGTTTTTGAGCAGCACCATCTTTCTTGTGTTTCCCTTCA
CTGGCATTGCGTGTTCCTATGGCTGGGTTCTCCTTGCTGTCTACCGCATGCACTCTGCAGAAGGGAGGAAAA
AGGCCTATTCGACCTGCAGCACCCACCTCACTGTAGTAACTTTCTACTATGCACCCTTTGCTTATACCTATC
TATGTCCAAGATCCCTCCGATCTCTGACAGAGGACAAGGTTCTGGCTGTTTTCTACACCATCCTCACCCCAA
TGCTCAACCCCATCATCTACAGCCTGAGAAACAAGGAGGTGATGGGGGCCCTGACACGAGTGATTCAGAATA
TCTTCTCGGTGAAAATGTAGACATAC

The disclosed NOV17a polypeptide (SEQ ID NO:70) encoded by SEQ ID NO:69 has 312 amino acid residues and is presented in Table 17B using the one-letter amino acid code.

TABLE 17B
Encoded NOV17a protein sequence.

| | |
|---|---|
| MENYNQTSTDFILLGLFPPSKIGLFLFILFVLIFLMALIGNLSMILLIFLDTHLHTPMYFLLSQ | (SEQ ID NO:70) |
| LSLIDLNYISTIVPKMASDFLYGNKSISFIGCGIQSFFFMTFAGAEALLLTSMAYDRYVAICFP | |
| LHYPIRMSKRMYVLMITGSWMIGSINSCAHTVYAFRIPYCKSRAINHFFCDVPAMLTLACTDTW | |
| VYEYTVFLSSTIFLVFPFTGIACSYGWVLLAVYRMHSARGRKKAYSTCSTHLTVVTFYYAPFAY | |
| TYLCPRSLRSLTEDKVLAVFYTILTPMLNPIIYSLRNKEVMGALTRVIQNIFSVKM. | |

A search of sequence databases reveals that the NOV17a amino acid sequence has 148 of 305 amino acid residues (48%) identical to, and 192 of 305 amino acid residues (62%) similar to, the 316 amino acid residue ptnr: TREMBLNEW-ACC:AAG45196 protein from Mus musculus (Mouse) (T2 OLFACTORY RECEPTOR) (E=8.0e⁻⁷³).

NOV17b

A disclosed NOV17b nucleic acid of 962 nucleotides (also referred to as CG56657-01) encoding a novel G-Protein Coupled Receptor-like protein is shown in Table 17C. An open reading frame was identified beginning with an ATG initiation codon at nucleotides 18-20 and ending with a TAG codon at nucleotides 954-956. The start and stop codons are shown in bold in Table 17C, and the 5′ and 3′ untranslated regions, if any, are underlined.

TABLE 17C
NOV17b nucleotide sequence

(SEQ ID NO:71)

GATCGTATGAATGCCCCATGGAAAATTACAATCAAACGTCAACTGATTTCATCTTATTGGGGCTGTTCCCAC
CATCAAAAATTGGCCTTTTCCTCTTCATTCTCTTTGTTCTCATTTTCCTAATGGCTCTAATTGGAAACCTAT
CCATGATTCTTCTCATCTTCTTGGACACCCATCTCCACACACCCATGTATTTCCTGCTTAGTCAGCTCTCCC
TCATTGACCTAAATTACATCTCTACGATTGTTCCTAAGATGGCTTCTGATTTTCTGTATGGAAACAAGTCTA
TCTCCTTCATTGGGTGTGGGATTCAGAGTTTCTTCTTCATGACTTTTGCAGGTGCAGAAGCGCTGCTCCTGA
CATCAATGGCCTATGATCGTTATGTGGCCATTTGCTTTCCTCTCCGCTATCCCATCCGTATGAGCAAAAGAA
TGTATGTGCTGATGATAACAGGATCTTGGATGATAGGCTCCATCAACTCTTGTGCTCACACAGTATATGCAT
TCCGTATCCCATATTGCAAGTCCAGAGCCATCAATCATTTTTTCTGTGATGTTCCAGCTATGTTGACATTAG
CCTGTACAGACACCTGGGTCTATGAGTACACAGTGTTTTTGAGCAGCACCATCTTTCTTGTGTTTCCCTTCA
CTGGCATTGCGTGTTCCTATGGCTGGGTTCTCCTTGCTGTCTACCGCATGCACTCTGCAGAAGGGAGGAAAA
AGGCCTATTCGACCTGCAGCACCCACCTCACTGTAGTAACTTTCTACTATGCACCCTTTGCTTATACCTATC
TATGTCCAAGATCCCTGCGATCTCTGACAGAGGACAAGGTTCTGGCTGTTTTCTACACCATCCTCACCCCAA
TGCTCAACCCCATCATCTACAGCCTGAGAAACAAGGAGGTGATGGGGGCCCTGACACGAGTGATTCAGAATA
TCTTCTCGGTGAAAATGTAGACATAC.

In a search of public sequence databases, the NOV17b nucleic acid sequence, localized to chromosome 4, has321 of 342 bases (93%) identical to a gb:GENBANK-ID:HSHTPRH07|acc:X64978.1 mRNA from Homo sapiens (H.sapiens mRNA HTPCRH07 for olfactory receptor) (E=2.9e⁻⁶²).

The disclosed NOV17b polypeptide (SEQ ID NO:72) encoded by SEQ ID NO:71 has 311 amino acid residues and is presented in Table 17D using the one-letter amino acid code. Signal P, Psort and/or Hydropathy results predict that NOV17b has no signal peptide and is likely to be localized to the plasma membrane with a certainty of 0.4600. Alternatively, NOV17b may also localize to the microbody (peroxisome) with a certainty of 0.2311, the endoplasmic reticulum (membrane) with a certainty of 0.1000, or in the endoplasmic reticulum (lumen) with a certainty of 0.1000. The most likely cleavage site for NOV17B is between positions 43 and 44: NLS-MI.

TABLE 17D
Encoded NOV17b protein sequence

(SEQ ID NO:72)
MENYNQTSTDFILLGLFPPSKIGLFLFILFVLIFLMALIGNLSMILLIFLDTHLHTPMYFLLSQLSLIDLNY
ISTIVPKMASDFLYGNKSISFIGCGIQSFFFMTFAGAEALLLTSMAYDRYVAICFPLRYPIRMSKRMYVLMI
TGSWMIGSINSCAHTVYAFRIPYCKSRAINHFFCDVPAMLTLACTDTWVYEYTVFLSSTIFLVFPFTGIACS
TGWVLLAVYRMHSAEGRKKAYSTCSTHLTVVTFYYAPFAYTYLCPRSLRSLTEDKVLAVFYTILTPMLNPII
YSLRNKEVMGALTRVIQNIFSVKM.

A search of sequence databases reveals that the NOV17b amino acid sequence has 148 of 305 amino acid residues (48%) identical to, and 191 of 305 amino acid residues (62%) similar to, the 316 amino acid residue ptnr:TREMBLNEW-ACC:AAG45196 protein from Mus musculus (Mouse) (T2 Olfactory Receptor) (E=8.0e⁻⁷³).

NOV17b is predicted to be expressed in at least the following tissues: Apical microvilli of the retinal pigment epithelium, arterial (aortic), basal forebrain, brain, Burkitt lymphoma cell lines, corpus callosum, cardiac (atria and ventricle), caudate nucleus, CNS and peripheral tissue, cerebellum, cerebral cortex, colon, cortical neurogenic cells, endothelial (coronary artery and umbilical vein) cells, palate epithelia, eye, neonatal eye, frontal cortex, fetal hematopoietic cells, heart, hippocampus, hypothalamus, leukocytes, liver, fetal liver, lung, lung lymphoma cell lines, fetal lymphoid tissue, adult lymphoid tissue, Those that express MHC II and III nervous, medulla, subthalamic nucleus, ovary, pancreas, pituitary, placenta, pons, prostate, putamen, serum, skeletal muscle, small intestine, smooth muscle (coronary artery in aortic) spinal cord, spleen, stomach, taste receptor cells of the tongue, testis, thalamus, and thymus tissue. This information was derived by determining the tissue sources of the sequences that were included in the invention including but not limited to SeqCalling sources, Public EST sources, Literature sources, and/or RACE sources.

NOV17c

A disclosed NOV17c nucleic acid of 883 nucleotides (also referred to as CG56659-01) encoding a novel G-Protein Coupled Receptor-like protein is shown in Table 17E. An open reading frame was identified beginning with an ATG initiation codon at nucleotides 44-46 and ending with a TAG codon at nucleotides 875-877. The start and stop codons are shown in bold in Table 17E, and the 5′ and 3′ untranslated regions, if any, are underlined.

TABLE 17E
NOV17c nucleotide sequence

(SEQ ID NO:73)

AATTGGCCTTTTCGTATTCACCCTCATTTTTCTCATTTTCCTAATGGCTCTAATTGGAAATCTATCCATGAT
TCTTCTCATCTTTTTGGACATCCATCTCCACACACCTATGTATTTCCTACTTAGTCAGCTCTCCCTCATTGA
CCTAAATTACATCTCCACCATTGTTCCAAAGATGGTTTATGATTTTCTGTATGGAAACAAGTCTATCTCCTT
CACTGGATGTGGGATTCAGAGTTTCTTCTTCTTGACTTTAGCAGTTGCAGAAGGGCTGCTCCTGACATCAAT
GGCCTATGATCGTTATGTGGCCATTTGCTTTCCTCTCCACTATCCCATCCGTATAAGCAAAAGAGTGTGTGT
GATGATGATAACAGGATCTTGGATGATAAGCTCTATCAACTCTTGTGCTCACACAGTATATGCACTCTGTAT
CCCATATTGCAAGTCCAGAGCCATCAATCATTTTTTCTGTGATGTTCCAGCTATGTTGACGCTAGCCTGCAC
AGACACTTGGGTCTATGAGAGCACAGTGTTTTTGAGCAGCACCATCTTTCTTGTGCTTCCTTTCACTGGTAT
TGCATGTTCCTATGGCCGGGTTCTCCTTGCTGTCTACCGCATGCACTCTGCAGAAGGGAGGAAGAAGGCCTA
TTCAACCTGTAGCACCCACCTCACTGTAGTGTCCTTCTACTATGCACCCTTTGCTTATACCTATGTACGTCC
AAGATCCCTGCGATCTCCAACAGAGGACAAGATTCTGGCTGTTTTCTACACCATCCTCACCCCAATGCTCAA
CCCCATCATCTACAGCCTGAGAAACAAGGAGGTGATGGGGGCCCTGACACAAGTGATTCAGAAAATCTTCTC
AGTGAAAATGTAGACATAC.

The disclosed NOV17c polypeptide (SEQ ID NO:74) encoded by SEQ ID NO:73 has 277 amino acid residues and is presented in Table 17F using the one-letter amino acid code.

TABLE 17F
Encoded NOV17c protein sequence

| | |
|---|---|
| MALIGNLSMTLLIFLDIHLHTPMYFLLSQLSLIDLNYISTIVPKMVYDFLYGNKSISFTGCGIQ | (SEQ ID NO:74) |
| SFFFLTLAVAEGLLLTSMAYDRYVAICFPLHYPIRISKRVCVMMITGSWMISSINSCAHTVYAL | |
| CIPYCKSRAINHFFCDVPAMLTLACTDTWVYESTVFLSSTIFLVLPFTGIACSYGRVLLAVYRM | |
| HSAEGRKKAYSTCSTHLTVVSFYYAPFAYTYVRPRSLRSPTEDKILAVFYTILTPMLNPIIYSLRNKEVM | |
| GALTQVIQKIFSVKM. | |

A search of sequence databases reveals that the NOV17c amino acid sequence has 139 of 272 amino acid residues (51%) identical to, and 181 of 272 amino acid residues (66%) similar to, the 316 amino acid residue ptnr: TPEMBLNEW-ACC:AAG45196 protein from Mus musculus (Mouse) (T2 OLFACTORY RECEPTOR) (E=4.0e⁻⁷¹).

NOV17d

A disclosed NOV17d nucleic acid of 926 nucleotides (also referred to as CG56659_{—}02) encoding a novel G-Protein Coupled Receptor-like protein is shown in Table 17G. An open reading frame was identified beginning with an ATG initiation codon at nucleotides 87-89 and ending with a TAG codon at nucleotides 918-920. The start and stop codons are shown in bold in Table 17G, and the 5′ and 3′ untranslated regions, if any, are underlined.

TABLE 17G
NOV17d nucleotide sequence

(SEQ ID NO:75)

CATCAACTGATTTCATCTTATTGGGGCTGTTCCCACAATCAAGAATTGGCCTTTTCGTATTCACCCTCATTT
TTCTCATTTTCCTAATGGCTCTAATTGGAAATCTATCCATGATTCTTCTCATCTTTTTGGACATCCATCTCC
ACACACCTATGTATTTCCTACTTAGTCAGCTCTCCCTCATTGACCTAAATTACATCTCCACCATTGTTCCAA
AGATGGTTTATGATTTTCTGTATGGAAACAAGTCTATCTCCTTCACTGGATGTGGGATTCAGAGTTTCTTCT
TCTTGACTTTAGCAGTTGCAGAAGGGCTGCTCCTGACATCAATGGCCTATGATCGTTATGTGGCCATTTGCT
TTCCTCTCCACTATCCCATCCGTATAAGCAAAAGAGTGTGTGTGATGATGATAACAGGATCTTGGATGATAA
GCTCTATCAACTCTTGTGCTCACACAGTATATGCACTCTGTATCCCATATTGCAAGTCCAGAGCCATCAATC
ATTTTTTCTGTGATGTTCCAGCTATGTTGACGCTAGCCTGCACAGACACTTGGGTCTATGAGAGCACAGTGT
TTTTGAGCAGCACCATCTTTCTTGTGCTTCCTTTCACTGGTATTGCATGTTCCTATGGCCGGGTTCTCCTTG
CTGTCTACCGCATGCACTCTGCAGAAGGGAGGAAGAAGGCCTATTCAACCTGTAGCACCCACCTCACTGTAG
TGTCCTTCTACTATGCACCCTTTGCTTATACCTATGTACGTCCAAGATCCCTGCGATCTCCAACAGAGGACA
AGATTCTGGCTGTTTTCTACACCATCCTCACCCCAATGCTCAACCCCATCATCTACAGCCTGAGAAACAAGG
AGGTGATGGGGGTCCTGACACAAGTGATTCAGAAAATCTTCTCAGTGAAAATGTAGACATAC.

In a search of public sequence databases, the NOV17d nucleic acid sequence has343 of 343 bases (100%) identical to a gb:GENBANK-ID:HSHTPRH07|acc:X64978.1 mRNA from Homo sapiens (H.sapiens mRNA HTPCRH07 for olfactory receptor) (E=5.4e⁻⁷¹).

The disclosed NOV17D polypeptide (SEQ ID NO:76) encoded by SEQ ID NO:75 has 277 amino acid residues and is presented in Table 17H using the one-letter amino acid code. Signal P, Psort and/or Hydropathy results predict that NOV17d has no signal peptide and is likely to be localized to the endoplasmic reticulum (membrane) with a certainty of 0.6850. Alternatively, NOV17d may also localize to the plasma membrane with a certainty of 0.6400, the Golgi body with a certainty of 0.4600, or in the endoplasmic reticulum (lumen) with a certainty of 0.1000. The most likely cleavage site for NOV17D is between positions 22 and 23: HTP-MY.

TABLE 17H
Encoded NOV17d protein sequence

(SEQ ID NO:76)

MALIGNLSMILLIFLDIHLHTPMYFLLSQLSLIDLNYISTIVPKMVYDFLYGNKSISFTGCGIQSFFFLTLA
VAEGLLLTSMAYDRYVAICFPLHYPIRISKRVCVMMITGSWMISSINSCAHTVYALCIPYCKSRAINHFFCD
VPAMLTLACTDTWVYESTVFLSSTIFLVLPFTGIACSYGRVLLAVYRMHSAEGRKKAYSTCSTHLTVVSFYY
APFAYTYVRPRSLRSPTEDKILAVFYTILTPMLNPIIYSLRNKEVMGVLTQVIQKIFSVKM.

A search of sequence databases reveals that the NOV17d amino acid sequence has 138 of 269 amino acid residues (51%) identical to, and 183 of 269 amino acid residues (68%) similar to, the 316 amino acid residue ptnr:SPTREMBL-ACC:Q9D3U9 protein from Mus musculus (Mouse) (4933433E02rik Protein) (E=3.9e⁻⁷¹).

NOV17d is predicted to be expressed in at least the following tissues: Apical microvilli of the retinal pigment epithelium, arterial (aortic), basal forebrain, brain, Burkitt lymphoma cell lines, corpus callosum, cardiac (atria and ventricle), caudate nucleus, CNS and peripheral tissue, cerebellum, cerebral cortex, colon, cortical neurogenic cells, endothelial (coronary artery and umbilical vein) cells, palate epithelia, eye, neonatal eye, frontal cortex, fetal hematopoietic cells, heart, hippocampus, hypothalamus, leukocytes, liver, fetal liver, lung, lung lymphoma cell lines, fetal lymphoid tissue, adult lymphoid tissue, Those that express MHC II and III nervous, medulla, subthalamic nucleus, ovary, pancreas, pituitary, placenta, pons, prostate, putamen, serum, skeletal muscle, small intestine, smooth muscle (coronary artery in aortic) spinal cord, spleen, stomach, taste receptor cells of the tongue, testis, thalamus, and thymus tissue. This information was derived by determining the tissue sources of the sequences that were included in the invention including but not limited to SeqCalling sources, Public EST sources, Literature sources, and/or RACE sources.

The disclosed NOV17a polypeptide has homology to the amino acid sequences shown in the BLASTP data listed in Table 17I.

TABLE 17I
BLAST results for NOV17a

| Gene Index/ | | Length | Identity | Positives | |
|---|---|---|---|---|---|
| Identifier | Protein/Organism | (aa) | (%) | (%) | Expect |
| >gi\|17445356\|ref\|XP_{—} | similar to | 312 | 312/312 | 312/312 | e−149 |
| 060561.1\| | OLFACTORY | | (100%) | (100%) | |
| (XM_060561) | RECEPTOR 2T1 | | | | |
| | (OLFACTORY | | | | |
| | RECEPTOR 1-25) | | | | |
| | (OR1-25) (H. | | | | |
| | sapiens) [Homo | | | | |
| | sapiens] | | | | |
| gi\|17445348\|ref\|XP_{—} | similar to | 533 | 199/233 | 206/233 | 1e−95 |
| 060559.1\| | OLFACTORY | | (85%) | (88%) | |
| (XM_060559) | RECEPTOR 2T1 | | | | |
| | (OLFACTORY | | | | |
| | RECEPTOR 1-25) | | | | |
| | (OR1-25) (H. | | | | |
| | sapiens) [Homo | | | | |
| | sapiens] | | | | |
| gi\|17437047\|ref\|XP_{—} | similar to | 472 | 149/299 | 211/299 | 5e−78 |
| 060312.1\| | OLFACTORY | | (49%) | (69%) | |
| (XM_060312) | RECEPTOR 2T1 | | | | |
| | (OLFACTORY | | | | |
| | RECEPTOR 1-25) | | | | |
| | (OR1-25) (H. | | | | |
| | sapiens) [Homo | | | | |
| | sapiens] | | | | |
| gi\|17437056\|ref\|XP_{—} | similar to | 695 | 155/295 | 209/295 | 1e−74 |
| 060314.1\| | OLFACTORY | | (52%) | (70%) | |
| (XM_060314) | RECEPTOR 2T1 | | | | |
| | (OLFACTORY | | | | |
| | RECEPTOR 1-25) | | | | |
| | (OR1-25) (H. | | | | |
| | sapiens) [Homo | | | | |
| | sapiens] | | | | |
| gi\|17456595\|ref\|XP_{—} | similar to | 638 | 138/296 | 193/296 | 1e−73 |
| 065073.1\| | olfactory | | (46%) | (64%) | |
| (XM_065073) | receptor (H. | | | | |
| | sapiens) [Homo | | | | |
| | sapiens] | | | | |

The homology between these and other sequences is shown graphically in the ClustalW analysis shown in Table 17J. In the ClustalW alignment of the NOV17 protein, as well as all other ClustalW analyses herein, the black outlined amino acid residues indicate regions of conserved sequence (i.e., regions that may be required to preserve structural or functional properties), whereas non-highlighted amino acid residues are less conserved and can potentially be altered to a much broader extent without altering protein structure or function.

[image]

[image]

[image]

[image]

[image]

Table 17F lists the domain description from DOMAIN analysis results against NOV17. This indicates that the NOV17 sequence has properties similar to those of other proteins known to contain this domain.

TABLE 17F
Domain Analysis of NOV17

gnL|PFam(pFam00001, 7tm_1, 7 transmembrane receptor (rhodopsin
family). (SEQ ID NO:810)
CD-Length=254 residues, 100.0% aligned
Score=99.4 bits (246), Expect=3e−22

| | | | |
|---|---|---|---|
| NOV17: | 40 | GNLSMILLIFLDTHLHTPMYFLLSQLSLIDLNYISTIVPKMASDFLYGNKSISFIGCGIQ | 99 |
| | | \|\|\| +\|\|+\| \| \|\| \| \|++ \|\| ++ \|+ \| + \|+ \| + | |
| Sbjct: | 1 | GNLLVILVILRTKKLRTPTNIFLLNLAVADLLFLLTLPPWALYYLVGGDWVFGDALCKLV | 60 |
| NOV17: | 100 | SFFFMTFAGAEALLLTSMAYDRYVAICFPLHYPIRMSKRMYVLMITGSWMIGSINSCAHT | 159 |
| Sbjct: | 61 | GALFVVNGYASILLLTAISIDRYLAIVHPLRYRRIRTPRRAKVLILLVWVLALLLSLPPL | 120 |
| NOV17: | 160 | VYAFRIPYCKSRAINHFFCDVPAMLTLACTDTWVYEYTVFLSSTIFLVFPFTGIACSYGW | 219 |
| | ++ + + + + \| \| \|\|+ + \| \| \| \| | | |
| Sbjct: | 121 | ---------SWLRTVEEGNTTVCLIDFPEESVKRSYVLLSTLVGFVLPLLVILVCYTR | 171 |
| NOV17: | 220 | VLLAV---------YRMHSAEGRKKAYSTCSTHLTVVTFYY----APFAYTYLCPRSLRS | 266 |
| | +\| + + \|+ \|\| \| + \| + + \| | | |
| Sbjct: | 172 | ILRTLRKRARSQRSLKRRSSSERKAAKMLLVVVVVFVLCWLPYHIVLLLDSLCLLSIWRV | 231 |
| NOV17: | 267 | LTEDKVLAVFYTILTPMLNPIIY 289 | |
| | \| ++ ++ + \|\|\|\|\|\| | | |
| Sbjct: | 232 | LPTALLITLWLAYVNSCLNPIIY 254 | |

G-Protein Coupled Receptor (GPCRs) have been identified as extremely large subfamily of G protein-coupled receptors in a number of species. These receptors share a seven transmembrane domain structure with many neurotransmitter and hormone receptors, and are likely to underlie the recognition and G-protein-mediated transduction of various signals. Previously, GPCR genes cloned in different species were from random locations in the respective genomes. The human GPCR genes are intron less and belong to four different gene subfamilies, displaying great sequence variability. These genes are dominantly expressed in olfactory epithelium.

Olfactory receptors (ORs) have been identified as extremely large subfamily of G protein-coupled receptors in a number of species. These receptors share a seven transmembrane domain structure with many neurotransmitter and hormone receptors, and are likely to underlie the recognition and G-protein-mediated transduction of odorant signals. Previously, OR genes cloned in different species were from random locations in the respective genomes. The human OR genes are intron less and belong to four different gene subfamilies, displaying great sequence variability. These genes are dominantly expressed in olfactory epithelium.

The disclosed NOV17 nucleic acid of the invention encoding a G-Protein Coupled Receptor-like protein includes the nucleic acid whose sequence is provided in Table 17A, 17C or a fragment thereof. The invention also includes a mutant or variant nucleic acid any of whose bases may be changed from the corresponding base shown in Table 17A or 17C while still encoding a protein that maintains its G-Protein Coupled Receptor-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 7 percent of the bases may be so changed.

The disclosed NOV17 protein of the invention includes the G-Protein Coupled Receptor-like protein whose sequence is provided in Table 17B or 17D. The invention also includes a mutant or variant protein any of whose residues may be changed from the corresponding residue shown in Table 17B or 17D while still encoding a protein that maintains its G-Protein Coupled Receptor-like activities and physiological functions, or a functional fragment thereof. In the mutant or variant protein, up to about 54 percent of the residues may be so changed.

The invention further encompasses antibodies and antibody fragments, such as F_{ab} or (F_{ab})₂, that bind immunospecifically to any of the proteins of the invention.

The above disclosed information suggests that this G-Protein Coupled Receptor-like protein (NOV17) is a member of a “G-Protein Coupled Receptor family”. Therefore, the NOV17 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV17 nucleic acids and proteins of the invention are useful in potential therapeutic applications implicated in developmental diseases, MHCII and III diseases (immune diseases), Taste and scent detectability Disorders, Burkitt's lymphoma, Corticoneurogenic disease, Signal Transduction pathway disorders, Retinal diseases including those involving photoreception, Cell Growth rate disorders; Cell Shape disorders, Feeding disorders;control of feeding; potential obesity due to over-eating; potential disorders due to starvation (lack of apetite), noninsulin-dependent diabetes mellitus (NIDDM1), bacterial, fungal, protozoal and viral infections (particularly infections caused by HIV-1 or HIV-2), pain, cancer (including but not limited to Neoplasm; adenocarcinoma; lymphoma; prostate cancer; uterus cancer), anorexia, bulimia, asthma, Parkinson's disease, acute heart failure, hypotension, hypertension, urinary retention, osteoporosis, Crohn's disease; multiple sclerosis; and Treatment of Albright Hereditary Ostoeodystrophy, angina pectoris, myocardial infarction, ulcers, asthma, allergies, benign prostatic hypertrophy, and psychotic and neurological disorders, including anxiety, schizophrenia, manic depression, delirium, dementia, severe mental retardation. Dentatorubro-pallidoluysian atrophy(DRPLA) Hypophosphatemic rickets, autosomal dominant (2) Acrocallosal syndrome and dyskinesias, such as Huntington's disease or Gilles de la Tourette syndrome, and/or other diseases and pathologies.

NOV17 nucleic acids and polypeptides are further useful in the generation of antibodies that bind immuno-specifically to the novel NOV17 substances for use in therapeutic or diagnostic methods. These antibodies may be generated according to methods known in the art, using prediction from hydrophobicity charts, as described in the “Anti-NOVX Antibodies” section below. The disclosed NOV17 protein has multiple hydrophilic regions, each of which can be used as an immunogen. These novel proteins can be used in assay systems for functional analysis of various human disorders, which will help in understanding of pathology of the disease and development of new drug targets for various disorders.

NOV18

NOV18 includes three novel G-Protein Coupled Receptor-like proteins disclosed below. The disclosed sequences have been named NOV18a and NOV18b.

NOV18a

A disclosed NOV18a nucleic acid of 1062 nucleotides (also referred to as CG56663-01) encoding a novel G-Protein Coupled Receptor-like protein is shown in Table 18A. An open reading frame was identified beginning with an ATG initiation codon at nucleotides 10-12 and ending with a TAA codon at nucleotides 948-950. The start and stop codons are shown in bold in Table 18A, and the 5′ and 3′ untranslated regions, if any, are underlined.

TABLE 18A
NOV18a nucleotide sequence

(SEQ ID NO:77)

TAGAGATGGATGGAACCAATGGCAGCACCCAAACCCATTCATCCTACTGGGATTCTCTGACCGACCCCATC
TGGAGAGGATCCTCTTTGTGGTCATCCTGATCGCGTACCTCCTGACCCTCGTAGGCAACACCACCATCATCC
TGGTGTCCCGGCTGGACCCCCACCTCCACACCCCCATGTACTTCTTCCTCGCCCACCTTTCCTTCCTGGACC
TCAGTTTCACCACCAGCTCCATCCCCCAGCTGCTCTACAACCTTAATGGATGTGACAAGACCATCAGCTACA
TGGGCTGTGCCATCCAGCTCTTCCTGTTCCTGGGTCTGGGTGGTGTGGAGTGCCTGCTTCTGGCTGTCATGG
CCTATGACCGGTGTGTGGCTATCTGCAAGCCCCTGCACTACATGGTGATCATGAACCCCAGGCTCTGCCGGG
GCTTGGTGTCAGTGACCTGGGGCTGTGGGGTGGCCAACTCCTTGGCCATGTCTCCTGTGACCCTGCGCTTAC
CCCGCTGTGGGCACCACGAGGTGGACCACTTCCTGCGTGAGATGCCCGCCCTGATCCGGATGGCCTGCGTCA
GCACTGTGGCCATCGAAGGCACCGTCTTTGTCCTGAAAAAAGGTGTTGTGCTGTCCCCCTTGGTGTTTATCC
TGCTCTCTTACAGCTACATTGTGAGGGCTGTGTTACAAATTCGGTCAGCATCAGGAAGGCAGAAGGCCTTCG
GCACCTGCGGCTCCCATCTCACTGTGGTCTCCCTTTTCTATGGAAACATCATCTACATGTACATGCAGCCAG
GAGCCAGTTCTTCCCAGGACCAGGGCATGTTCCTCATGCTCTTCTACAACATTGTCACCCCCCTCCTCAATC
CTCTCATCTACACCCTCAGAAACAGAGAGGTGAAGGGGGCACTGGGAAGGTTGCTTCTGGGGAAGAGAGAGC
TAGGAAAGGAGTAAAGGCATCTCCACCTGACTTCACTTCCATCCAGGGCCACTGGCAGCATCTGGAACGGCT
GAATTCCAGCTGATATTAGCCCACGACTCCCAACTTGCCTTTTTCTGGACTTTT.

The disclosed NOV18a polypeptide (SEQ ID NO:78) encoded by SEQ ID NO:77 has 314 amino acid residues and is presented in Table 18B using the one-letter amino acid code.

TABLE 18B
Encoded NOV18a protein sequences

| | |
|---|---|
| MDGTNGSTQTHFILLGFSDRPHLERILFVVILIAYLLTLVGNTTIILVSRLDPHLHTPMYFFLA | (SEQ ID NO:78) |
| HLSFLDLSFTTSSIPQLLYNLNGCDKTISYMGCAIQLFLFLGLFFVECLLLAVMAYDRCVAICK | |
| PLHYMVIMNPRLCRGLVSVTWGCGVANSLAMSPVTLRLPRCGHHEVDHFLREMPALIRMACVST | |
| VAIEGTVFVLKKGVVLSPLVFILLSYSYIVRAVLQIRSASGRQKAFGTCGSHLTVVSLFYGNII | |
| YMYMQPGASSSQDQGMFLMLFYNIVTPLLNPLIYTLRNREVKGALGRLLLGKRELGKE. | |

A search of sequence databases reveals that the NOV18a amino acid sequence has 194 of 237 amino acid residues (81%) identical to, and 215 of 237 amino acid residues (90%) similar to, the 237 amino acid residue ptnr: SPTREMBL-ACC:Q9R0G5 protein from Marmota marmota (European marmot) (Olfactory Receptor) (E=3.5e⁻¹⁰²).

NOV18b

A disclosed NOV18b nucleic acid of 1062 nucleotides (also referred to as CG56663-02) encoding a novel G-Protein Coupled Receptor-like protein is shown in Table 18C. An open reading frame was identified beginning with an ATG initiation codon at nucleotides 6-8 and ending with a TAA codon at nucleotides 948-950. The start and stop codons are shown in bold in Table 18C, and the 5′ and 3′ untranslated regions, if any, are underlined.

TABLE 18C
NOV18b nucleotide sequence

(SEQ ID NO:79)

TAGAGATGGATGGAACCAATGGCAGCACCCAAACCCATTTCATCCTACTGGGATTCTCTGACCGACCCCATC
TGGAGAGGATCCTCTTTGTGGTCATCCTGATCGCGTACCTCCTGACCCTCGTAGGCAACACCACCATCATCC
TGGTGTCCCGGCTGGACCCCCACCTCCACACCCCCATGTACTTCTTCCTCGCCCACCTTTCCTTCCTGGACC
TCAGTTTCACCACCAGCTCCATCCCCCAGCTGCTCTACAACCTTAATGGATGTGACAAGACCATCAGCTACA
TGGGCTGTGCCATCCAGCTCTTCCTGTTCCTGGGTCTGGGTGGTGTGGAGTGCCTGCTTCTGGCTGTCATCC
CCTATGACCGGTGTGTGGCTATCTGCAAGCCCCTGCACTACATGGTGATCATGAACCCCAGGCTCTGCCGGG
GCTTGGTGTCAGTGACCTGGGGCTGTGGGGTGGCCAACTCCTTGGCCATGTCTCCTGTGACCCTGCGCTTAC
CCCGCTGTGGGCACCACGAGGTGGACCACTTCCTGCGTGAGATGCCCGCCCTGATCCGGATGGCCTGCGTCA
GCACTGTGGCCATCGACGGCACCGTCTTTGTCCTGGCGGTGGGTGTTGTGCTGTCCCCCTTGGTGTTTATCC
TGCTCTCTTACAGCTACATTGTGAGGGCTGTGTTACAAATTCGGTCAGCATCAGGAAGGCAGAAGGCCTTCG
GCACCTGCGGCTCCCATCTCACTGTGGTCTCCCTTTTCTATGGAAACATCATCTACATGTACATGCAGCCAG
GAGCCAGTTCTTCCCAGGACCAGGGCATGTTCCTCATGCTCTTCTACAACATTGTCACCCCCCTCCTCAATC
CTCTCATCTACACCCTCAGAAACAGAGAGGTGAAGGGGGCACTGGGAAGGTTGCTTTTGGGGAAGAGAGAGC
TAGGAAAGGAGTAAAGGCATCTCCACCTGACTTCACTTCCATCCAGGGCCACTGGCAGCATCTGGAACGGCT
GAATTCCAGCTGATATTAGCCCACGACTCCCAACTTGCCTTTTTCTGGACTTTT.

In a search of public sequence databases, the NOV18b nucleic acid sequence has600 of 710 bases (84%) identical to a gb:GENBANK-ID:AX008326|acc:AX008326.1 mRNA from Marmota marmota (Sequence 24 from Patent WO9967282) (E=8.8e⁻¹⁰⁹).

The disclosed NOV18D polypeptide (SEQ ID NO:80) encoded by SEQ ID NO:79 has 314 amino acid residues and is presented in Table 18D using the one-letter amino acid code. Signal P, Psort and/or Hydropathy results predict that NOV18b has A signal peptide and is likely to be localized to the plasma membrane with a certainty of 0.6000. Alternatively, NOV18b may also localize to the Golgi body with a certainty of 0.4000, the endoplasmic reticulum (membrane) with a certainty of 0.3000, or in the endoplasmic reticulum (lumen) with a certainty of 0.3000. The most likely cleavage site for NOV18b is between positions 42 and 43: LVG-NT.

TABLE 18D
Encoded NOV18b protein sequence

(SEQ ID NO:80)

MDGTNGSTQTHFILLGFSDRPHLERILFVVILIAYLLTLVGNTTIILVSRLDPHLHTPMYFFLAHLSFLDLS
FTTSSIPQLLYNLNGCDKTISYMGCAIQLFLFLGLGGVECLLLAVMAYDRCVAICKPLHYMVIMNPRLCRGL
VSVTWGCGVANSLAMSPVTLRLPRCGHHEVDHFLREMPALIRMACVSTVAIDGTVFVLAVGVVLSPLVFILL
SYSYIVRAVLQIRSASGRQKAFGTCGSHLTVVSLFYGNIIYMYMQPGASSQDQGMFLMLFYNIVTPLLNPL
IYTLRNREVKGALGRLLLGKRELGKE.

A search of sequence databases reveals that the NOV18b amino acid sequence has 183 of 305 amino acid residues (60%) identical to, and 237 of 305 amino acid residues (77%) similar to, the 320 amino acid residue ptnr:SPTREMBL-ACC:Q9Y3N9 protein from Homo sapiens (Human) (DJ88J8.1 (Novel 7 Transmembrane Receptor (Rhodopsin Family) (Olfactory Receptor Like) Protein) (HS6M1-15))) (E=2.8e⁻⁹⁸).

NOV18b is predicted to be expressed in at least the following tissues: Apical microvilli of the retinal pigment epithelium, arterial (aortic), basal forebrain, brain, Burkitt lymphoma cell lines, corpus callosum, cardiac (atria and ventricle), caudate nucleus, CNS and peripheral tissue, cerebellum, cerebral cortex, colon, cortical neurogenic cells, endothelial (coronary artery and umbilical vein) cells, palate epithelia, eye, neonatal eye, frontal cortex, fetal hematopoietic cells, heart, hippocampus, hypothalamus, leukocytes, liver, fetal liver, lung, lung lymphoma cell lines, fetal lymphoid tissue, adult lymphoid tissue, Those that express MHC II and III nervous, medulla, subthalamic nucleus, ovary, pancreas, pituitary, placenta, pons, prostate, putamen, serum, skeletal muscle, small intestine, smooth muscle (coronary artery in aortic) spinal cord, spleen, stomach, taste receptor cells of the tongue, testis, thalamus, and thymus tissue. This information was derived by determining the tissue sources of the sequences that were included in the invention including but not limited to SeqCalling sources, Public EST sources, Literature sources, and/or RACE sources.

The disclosed NOV18a polypeptide has homology to the amino acid sequences shown in the BLASTP data listed in Table 18E.

TABLE 18E
BLAST results for NOV18a

| Gene Index/ | | Length | Identity | Positives | |
|---|---|---|---|---|---|
| Identifier | Protein/Organism | (aa) | (%) | (%) | Expect |
| gi\|17445344\|ref\|XP_{—} | similar to | 314 | 314/314 | 314/314 | e−164 |
| 060558.1\| | olfactory | | (100%) | (100%) | |
| (XM_060558) | receptor (H. | | | | |
| | sapiens) [Homo | | | | |
| | sapiens] | | | | |
| gi\|5901478\|gb\|AAD55304.1\| | olfactory | 237 | 194/237 | 215/237 | 2e−99 |
| AF044033_1 | receptor [Marmota | | (81%) | (89%) | |
| (AF044033) | marmota] | | | | |
| gi\|13624329\|ref\|NP_{—} | olfactory | 320 | 184/305 | 236/305 | 1e−94 |
| 112165.1\| | receptor, family | | (60%) | (77%) | |
| (NM_030903) | 2, subfamily W, | | | | |
| | member 1 [Homo | | | | |
| | sapiens] | | | | |
| gi\|12054431\|emb\|CAC20523.1\| | olfactory | 320 | 184/305 | 236/305 | 1e−94 |
| (AJ302603) | receptor [Homo | | (60%) | (77%) | |
| | sapiens] | | | | |
| gi\|12054429\|emb\|CAC20522.1\| | olfactory | 320 | 184/305 | 235/305 | 2e−94 |
| (AJ302602) | receptor [Homo | | (60%) | (76%) | |
| | sapiens] | | | | |

The homology between these and other sequences is shown graphically in the ClustalW analysis shown in Table 18F. In the ClustalW alignment of the NOV18 protein, as well as all other ClustalW analyses herein, the black outlined amino acid residues indicate regions of conserved sequence (i.e., regions that may be required to preserve structural or functional properties), whereas non-highlighted amino acid residues are less conserved and can potentially be altered to a much broader extent without altering protein structure or function.

[image]

[image]

Tables 18G lists the domain descriptions from DOMAIN analysis results against NOV18. This indicates that the NOV18 sequence has properties similar to those of other proteins known to contain this domain.

TABLE 18G
Domain Analysis of NOV18

gnl|Pfam|pfam00001, 7tm_, 7 transmembrane receptor (rhodopsin
family). (SEQ ID NO:810)
CD-Length=254 residues, 100.0% aligned
Score=95.1 bits (235), Expect=5e−21

| | | | |
|---|---|---|---|
| NOV18: | 41 | GNTTIILVSRLDPHLHTPMYFFLAHLSFLDLSFTTSSIPQLLYNLNGCDKTISYMGCAIQ | 100 |
| | | \|\| +\|\|\| \| \|\| \|\| +\|+ \|\| \| + \| \|\| \| \| \| \| + | |
| Sbjct: | 1 | GNLLVILVILRTKKLRTPTNIFLLNLAVADLLFLLTLPPWALYYLVGGDWVFGDALCKLV | 60 |
| NOV18: | 101 | LFLFLGLGGVECLLLAVMAYDRCVAICKPLHYMVIMNPRLCRGLVSVTWGCGVANSLAMS | 160 |
| | | \|\|+ \| \|\|\| ++ \|\| +\|\| \|\| \| \| \|\| + \|+ + \| + \|\| | |
| Sbjct: | 61 | GALFVVNGYASILLLTAISIDRYLAIVHPLRYRRIRTPRRAKVLILLVWVLALLLSLP-- | 118 |
| NOV18: | 161 | PVTLRLPRCGHHEVDHFLREMPALIRMACVSTVAIEGTVFVLKKGVVLSPLVFILLSYSY | 220 |
| | | \|+ \| + + \|\|\| \|\|+ \|\|+ \|+ | |
| Sbjct: | 119 | PLLFSWLRTVEEGNTTVCLIDFPEESVKRSYVLLSTLVGFVL-------PLLVILVCYTR | 171 |
| NOV18: | 221 | IVRAV---------LQIRSASGRQKAFGTCGSHLTVVSLFYG----NIIYMYMQPGASSS | 267 |
| | | \|+\| +---------\|+ \|\|+\| \|+ \| + \| + ++ | |
| Sbjct: | 172 | ILRTLRKRARSQRSLKRRSSSERKAAKMLLVVVVVFVLCWLPYHIVLLLDSLCLLSIWRV | 231 |
| NOV18: | 268 | QDQGMFLMLFYNIVTPLLNPLIY | 290 |
| | | + + \|+ \| \|\|\|+\|\| | |

Sbjct: 232 LPTALLITLWLAYVNSCLNPIIY 254

G-Protein Coupled Receptor (GPCRs) have been identified as extremely large subfamily of G protein-coupled receptors in a number of species. These receptors share a seven transmembrane domain structure with many neurotransmitter and hormone receptors, and are likely to underlie the recognition and G-protein-mediated transduction of various signals. Previously, GPCR genes cloned in different species were from random locations in the respective genomes. The human GPCR genes are intron less and belong to four different gene subfamilies, displaying great sequence variability. These genes are dominantly expressed in olfactory epithelium.

Olfactory receptors (ORs) have been identified as extremely large subfamily of G protein-coupled receptors in a number of species. These receptors share a seven transmembrane domain structure with many neurotransmitter and hormone receptors, and are likely to underlie the recognition and G-protein-mediated transduction of odorant signals. Previously, OR genes cloned in different species were from random locations in the respective genomes. The human OR genes are intron less and belong to four different gene subfamilies, displaying great sequence variability. These genes are dominantly expressed in olfactory epithelium.

The disclosed NOV18 nucleic acid of the invention encoding a G-Protein Coupled Receptor-like protein includes the nucleic acid whose sequence is provided in Table 18A, 20C or a fragment thereof. The invention also includes a mutant or variant nucleic acid any of whose bases may be changed from the corresponding base shown in Table 18A or 20C while still encoding a protein that maintains its G-Protein Coupled Receptor-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 16 percent of the bases may be so changed.

The disclosed NOV18 protein of the invention includes the G-Protein Coupled Receptor-like protein whose sequence is provided in Table 18B or 20D. The invention also includes a mutant or variant protein any of whose residues may be changed from the corresponding residue shown in Table 18B or 20D while still encoding a protein that maintains its G-Protein Coupled Receptor-like activities and physiological functions, or a functional fragment thereof. In the mutant or variant protein, up to about 40 percent of the residues may be so changed.

The invention further encompasses antibodies and antibody fragments, such as F_{ab} or (F_{ab})₂, that bind immunospecifically to any of the proteins of the invention.

The above disclosed information suggests that this G-Protein Coupled Receptor-like protein (NOV18) is a member of a “G-Protein Coupled Receptor family”. Therefore, the NOV18 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV18 nucleic acids and proteins of the invention are useful in potential therapeutic applications implicated in developmental diseases, MHCII and III diseases (immune diseases), Taste and scent detectability Disorders, Burkitt's lymphoma, Corticoneurogenic disease, Signal Transduction pathway disorders, Retinal diseases including those involving photoreception, Cell Growth rate disorders; Cell Shape disorders, Feeding disorders;control of feeding; potential obesity due to over-eating; potential disorders due to starvation (lack of apetite), noninsulin-dependent diabetes mellitus (NIDDM1), bacterial, fungal, protozoal and viral infections (particularly infections caused by HIV-1 or HIV-2), pain, cancer (including but not limited to Neoplasm; adenocarcinoma; lymphoma; prostate cancer; uterus cancer), anorexia, bulimia, asthma, Parkinson's disease, acute heart failure, hypotension, hypertension, urinary retention, osteoporosis, Crohn's disease; multiple sclerosis; and Treatment of Albright Hereditary Ostoeodystrophy, angina pectoris, myocardial infarction, ulcers, asthma, allergies, benign prostatic hypertrophy, and psychotic and neurological disorders, including anxiety, schizophrenia, manic depression, delirium, dementia, severe mental retardation. Dentatorubro-pallidoluysian atrophy(DRPLA) Hypophosphatemic rickets, autosomal dominant (2) Acrocallosal syndrome and dyskinesias, such as Huntington's disease or Gilles de la Tourette syndrome, and/or other diseases and pathologies.

NOV18 nucleic acids and polypeptides are further useful in the generation of antibodies that bind immuno-specifically to the novel NOV18 substances for use in therapeutic or diagnostic methods. These antibodies may be generated according to methods known in the art, using prediction from hydrophobicity charts, as described in the “Anti-NOVX Antibodies” section below. The disclosed NOV18 protein has multiple hydrophilic regions, each of which can be used as an immunogen. These novel proteins can be used in assay systems for functional analysis of various human disorders, which will help in understanding of pathology of the disease and development of new drug targets for various disorders.

NOV19

NOV19 includes three novel G-Protein Coupled Receptor-like proteins disclosed below. The disclosed sequences have been named NOV19a and NOV19b.

NOV19a

A disclosed NOV19a nucleic acid of 1046 nucleotides (also referred to as CG56665-01) encoding a novel G-Protein Coupled Receptor-like protein is shown in Table 19A. An open reading frame was identified beginning with an ATG initiation codon at nucleotides 14-16 and ending with a TGA codon at nucleotides 1019-1021. The start and stop codons are shown in bold in Table 19A, and the 5′ and 3′ untranslated regions, if any, are underlined.

TABLE 19A
NOV19a nucleotide sequence

(SEQ ID NO:81)

TCAACATTATTACATGAACATTTCAGATGTCATCTCCTTTGATATTTTGGTTTCAGCCATGAAAACAGGAAA
TCAAAGTTTTGGGACAGATTTTCTACTTGTTGGTCTTTTCCAATATGGCTGGATAAACTCTCTTCTCTTTGT
CGTCATTGCCACCCTCTTTACAGTTGCTCTGACAGGAAATATCATGCTGATCCACCTCATTCGACTGAACAC
CAGACTCCACACTCCAATGTACTTTCTGCTCAGTCAGCTCTCCATCGTTGACCTCATGTACATCTCCACCAC
AGTGCCCAAGATGGCAGTCAGCTTCCTCTCACAGAGTAAGACCATTAGATTTTTGGGCTGTGAGATTCAAAC
GTATGTGTTCTTGGCCCTTGGTGGAACTGAAGCCCTTCTCCTTGGTTTTATGTCTTATGATCGCTATGTAGC
TATCTGTCACCCTTTACATTATCCTATGCTTATGAGCAAGAAGATCTGCTGCCTCATCCTTGCATGTGCATG
GGCCAGTGGTTCTATCAATGCTTTCATACATACATTGTATGTGTTTCAGCTTCCATTCTGTAGGTCTCGGCT
CATTAACCACTTTTTCTGTGAAGTTCCAGCTCTACTATCATTGGTGTGTCAGGACACCTCCCAGTATGAGTA
TACAGTCCTCCTGAGTGGACTTATTATCTTGCTACTACCATTCCTAGCCATTCTGGCTTCCTATGCTCGTGT
GCTTATTGTGGTATTCCAGATGAGCTCAGGAAAAGGACAGGCAAAAGCTGTTTCCACTTGTTCCTCCCACCT
GATTGTGGCAAGCCTGTTCTATGCAACCACTCTCTTTACCTACACAAGGCCACACTCCTTGCGTTCCCCTTC
ACGGGATAAGGCGGTGGCAGTATTTTACACCATTGTCACACCTCTACTGAACCCATTTATCTACAGCCTGAG
AAATAAGGAAGTGACGGGGGCAGTGAGGAGACTGTTGGGATATTGGATATGCTGTAGAAAATATGACTTCAG
ATCTCTGTATTGATTGAGCATTAACAACATAAAAAGCT.

The disclosed NOV19a polypeptide (SEQ ID NO:82) encoded by SEQ ID NO:81 has 335 amino acid residues and is presented in Table 19B using the one-letter amino acid code.

TABLE 19B
Encoded NOV19a protein sequence

| | |
|---|---|
| MNISDVISFDILVSAMKTGNQSFGTDFLLVGLFQYGWINSLLFVVIATLFTVALTGNIMLIHLI | (SEQ ID NO:82) |
| RLNTRLHTPMYFLLSQLSIVDLMYISTTVPKMAVSFLSQSKTIRFLGCEIQTYVFLALGGTEAL | |
| LLGFMSYDRYVAICHPLHYPMLMSKKICCLMVACAWASGSINAFIHTLYVFQLPFCRSRLINHF | |
| FCEVPALLSLVCQDTSQYEYTVLLSGLIILLLPFLAILASYARVLIVVFQMSSGKGQAKAVSTC | |
| SSHLIVASLFYATTLFTYTRPHSLRSPSRDKAVAVFYTIVTPLLNPFIYSLRNKEVTGAVRRLLGYWIC | |
| CRKYDFRSLY. | |

A search of sequence databases reveals that the NOV19a amino acid sequence has 155 of 309 amino acid residues (50%) identical to, and 199 of 309 amino acid residues (64%) similar to, the 316 amino acid residue ptnr: TREMBLNEW-ACC:AAG45196 protein from Mus musculus (Mouse) (T2 Olfactory Receptor) (E=9.3e⁻⁷⁹).

NOV19b

A disclosed NOV19b nucleic acid of 1046 nucleotides (also referred to as CG56665-02) encoding a novel G-Protein Coupled Receptor-like protein is shown in Table 19C. An open reading frame was identified beginning with an ATG initiation codon at nucleotides 59-60 and ending with a TGA codon at nucleotides 1019-1021. The start and stop codons are shown in bold in Table 19C, and the 5′ and 3′ untranslated regions, if any, are underlined.

TABLE 19C
NOV19b nucleotide sequence.

(SEQ ID NO:83)

TCAACATTATTACATGAACATTTCAGATGTCATCTCCTTTGATATTTTGGTTTCAGCCATGAAAACAGGAAA
TCAAAGTTTTGGGACAGATTTTCTACTTGTTGGTCTTTTCCAATATGGCTGGATAAACTCTCTTCTCTTTGT
CGTCATTGCCACCCTCTTTACAGTTGCTCTGACAGGAAATATCATGCTGATCCACCTCATTCGACTGAACAC
CAGACTCCACACTCCAATGTACTTTCTGCTCAGTCAGCTCTCCATCGTTGACCTCATGTACATCTCCACCAC
AGTGCCCAAGATGGCAGTCAGCTTCCTCTCACAGAGTAAGACCATTAGATTTTTGGGCTGTGAGATTCAAAC
GTATGTGTTCTTGGCCCTTGGTGGAACTGAAGCCCTTCTCCTTGGTTTTATGTCTTATGATCGCTATGTAGC
TATCTGTCACCCTTTACATTATCCTATGCTTATGAGCAAGAAGATCTGCTGCCTCATGGTTGCATGTGCATG
GGCCAGTGGTTCTATCAATGCTTTCATACATACATTGTATGTGTTTCAGCTTCCATTCTGTAGGTCTCGGCT
CATTAACCACTTTTTCTGTGAAGTTCCAGCTCTACTATCATTGATGTGTCAGGACACCTCCCAGTATGAGTA
TACAGTCCTCCTGAGTGGACTTATTATCTTGCTACTACCATTCCTAGCCATTCTGGCTTCCTATGCTCGTGT
GCTTATTGTGGTATTCCAGATGAGCTCAGGAAAAGGACAGGCAAAACCTGTTTCCACTTGTTCCTCCCACCT
GATTGTGGCAAGCCTGTTCTATGCAACCACTCTCTTTACCTACACAAGGCCACACTCCTTGCGTTCCCCTTC
ACGGGATAAGGCGGTGGCAGTATTTTACACCATTGTCACACCTCTACTGAACCCATTTATCTACAGCCTGAG
AAATAAGGAAGTGACGGGGGCAGTGAGGAGACTGTTGGGATATTGGATATGCTGTAGAAAATATGACTTCAG
ATCTCTGTATTGATTGAGCATTAACAACATAAAAAGCT

In a search of public sequence databases, the NOV19b nucleic acid sequence has 592 of 910 bases (65%) identical to a gb:GENBANK-ID:GGCOR4GEN|acc:X94744.1 mRNA from Gallus gallus (G.gallus cor4 DNA for olfactory receptor 4) (E=7.8e⁻⁴⁸).

The disclosed NOV19b polypeptide (SEQ ID NO:84) encoded by SEQ ID NO:83 has 320 amino acid residues and is presented in Table 19D using the one-letter amino acid code. Signal P, Psort and/or Hydropathy results predict that NOV19b has A signal peptide and is likely to be localized to the plasma membrane with a certainty of 0.4600. Alternatively, NOV19b may also localize to the microbody (peroxisome) with a certainty of 0.2188, the endoplasmic reticulum (membrane) with a certainty of 0.1000, or in the endoplasmic reticulum (lumen) with a certainty of 0.1000. The most likely cleavage site for NOV19b is between positions 40 and 41: ALT-GN.

TABLE 19D
Encoded NOV19b protein sequence.

(SEQ ID NO:84)

MKTGNQSFGTDFLLVGLFQYGWINSLLFVVIATLFTVALTGNIMLIHLTRLNTRLHTPMYFLLSQLSIVDLM
YISTTVPKMAVSFLSQSKTIRFLGCEIQTYVFLALGGTEALLLGFMSYDRYVAICHPLHYPMLMSKKICCLM
VACAWASGSINAFIHTLYVFQLPFCRSRLINHFFCEVPALLSLMCQDTSQYEYTVLLSGLIILLLPFLAILA
SYARVLIVVFQMSSGKGQAKAVSTCSSHLIVASLFYATTLFTYTRPHSLRSPSRDKAVAVFYTIVTPLLNPF
IYSLRNKEVTGAVRRLLGYWICCRKYDFRSLY

A search of sequence databases reveals that the NOV19b amino acid sequence has 155 of 306 amino acid residues (50%) identical to, and 198 of 306 amino acid residues (64%) similar to, the 316 amino acid residue ptnr:TREMBLNEW-ACC:BAB30304 protein from Mus musculus (Mouse) (Adult Male Testis cDNA, Riken Full-Length Enriched Library, Clone:4932441h21, Full Insert Sequence) (E=1.3e⁻⁷⁹).

NOV19b is predicted to be expressed in at least the following tissues: Apical microvilli of the retinal pigment epithelium, arterial (aortic), basal forebrain, brain, Burkitt lymphoma cell lines, corpus callosum, cardiac (atria and ventricle), caudate nucleus, CNS and peripheral tissue, cerebellum, cerebral cortex, colon, cortical neurogenic cells, endothelial (coronary artery and umbilical vein) cells, palate epithelia, eye, neonatal eye, frontal cortex, fetal hematopoietic cells, heart, hippocampus, hypothalamus, leukocytes, liver, fetal liver, lung, lung lymphoma cell lines, fetal lymphoid tissue, adult lymphoid tissue, Those that express MHC II and III nervous, medulla, subthalamic nucleus, ovary, pancreas, pituitary, placenta, pons, prostate, putamen, serum, skeletal muscle, small intestine, smooth muscle (coronary artery in aortic) spinal cord, spleen, stomach, taste receptor cells of the tongue, testis, thalamus, and thymus tissue. This information was derived by determining the tissue sources of the sequences that were included in the invention including but not limited to SeqCalling sources, Public EST sources, Literature sources, and/or RACE sources.

The disclosed NOV19a polypeptide has homology to the amino acid sequences shown in the BLASTP data listed in Table 19E.

TABLE 19E
BLAST results for NOV19a

| Gene Index/ | | Length | Identity | Positives | |
|---|---|---|---|---|---|
| Identifier | Protein/Organism | (aa) | (%) | (%) | Expect |
| gi\|17445348\|ref\|XP_{—} | similar to | 533 | 300/301 | 301/301 | e−143 |
| 060559.1\| | OLFACTORY | | (99%) | (99%) | |
| (XM_060559) | RECEPTOR 2T1 | | | | |
| | (OLFACTORY | | | | |
| | RECEPTOR 1-25) | | | | |
| | (OR1-25) (H. | | | | |
| | sapiens) [Homo | | | | |
| | sapiens] | | | | |
| gi\|17437056\|ref\|XP_{—} | similar to | 695 | 169/310 | 224/310 | 5e−84 |
| 060314.1\| | OLFACTORY | | (54%) | (71%) | |
| (XM_060314) | RECEPTOR 2T1 | | | | |
| | (OLFACTORY | | | | |
| | RECEPTOR 1-25) | | | | |
| | (OR1-25) (H. | | | | |
| | sapiens) [Homo | | | | |
| | sapiens] | | | | |
| gi\|17445356\|ref\|XP_{—} | similar to | 312 | 172/305 | 223/305 | 3e−80 |
| 060561.1\| | OLFACTORY | | (56%) | (72%) | |
| (XM_060561) | RECEPTOR 2T1 | | | | |
| | (OLFACTORY | | | | |
| | RECEPTOR 1-25) | | | | |
| | (OR1-25) (H. | | | | |
| | sapiens) [Homo | | | | |
| | sapiens] | | | | |
| gi\|17456595\|ref\|XP_{—} | similar to | 638 | 142/292 | 188/292 | 7e−78 |
| 065073.1\| | olfactory | | (48%) | (63%) | |
| (XM_065073) | receptor (H. | | | | |
| | sapiens) [Homo | | | | |
| | sapiens] | | | | |
| gi\|17475192\|ref\|XP_{—} | similar to | 315 | 154/299 | 209/299 | 2e−77 |
| 062796.1\| | olfactory | | (51%) | (69%) | |
| (XM_062796) | receptor (H. | | | | |
| | sapiens) [Homo | | | | |
| | sapiens] | | | | |

The homology between these and other sequences is shown graphically in the ClustalW analysis shown in Table 19F. In the ClustalW alignment of the NOV19 protein, as well as all other ClustalW analyses herein, the black outlined amino acid residues indicate regions of conserved sequence (i.e., regions that may be required to preserve structural or functional properties), whereas non-highlighted amino acid residues are less conserved and can potentially be altered to a much broader extent without altering protein structure or function.

[image]

[image]

[image]

Table 19G lists the domain description from DOMAIN analysis results against NOV19. This indicates that the NOV19 sequence has properties similar to those of other proteins known to contain this domain.

TABLE 19G
Domain Analysis of NOV19

gnl|Pfam|pfam00001, 7tm_1, 7 transmembrane receptor (rhodopsin
family). (SEQ ID NO:810)
CD-Length = 254 residues, 100.0% aligned
Score = 91.3 bits (225), Expect = 8e−20

| | | | |
|---|---|---|---|
| NOV19: | 56 | GNIMLIHLIRLNTRLHTPMYFLLSQLSIVDLMYISTTVPKMAVSFLSQSKTIRFLGCEIQ | 115 |
| | | \|\|+++\| +\| +\| \|\| \|++ \|\|+++ \| \| + \|++ | |
| Sbjct: | 1 | GNLLVILVILRTKKLRTPTNIFLLNLAVADLLFLLTLPPWALYYLVGGDWVFGDALCKLV | 60 |
| NOV19: | 116 | TYVFLALGGTEALLLGFMSYDRYVAICHPLHYPMLMSKKICCLMVACAWASGSINAFIHT | 175 |
| | | +\|+ \| \|\|\| +\| \|\|\|+\|\| \|\|\| \| + + + +++ \| + + | |
| Sbjct: | 61 | GALFVVNGYASILLLTAISIDRYLAIVHPLRYRRIRTPRRAKVLILLVWVLALLLSLPPL | 120 |
| NOV19: | 176 | LYVFQLPFCRSRLINHFFCEVPALLSLVCQDTSQYEYTVLLSGLITLLLPFLAILASYAR | 235 |
| | | \|+ + + \| \|\| +\|++ + | |
| Sbjct: | 121 | LFSWLRTVEEGNTTVCLIDFPEESVKRSYVLLSTLVGFVLPLLVILVCYTRILRTLRKRA | 180 |
| NOV19: | 236 | VLIVVFQMSSGKGQAKAVSTCSSHLIVASLFY----ATTLFTYTRPHSLRSPSRDKAVAV | 291 |
| | | + \| + \| ++ + \| + \| + + | |
| Sbjct: | 181 | RSQRSLKRRSSSERKAAKMLLVVVVVFVLCWLPYHIVLLLDSLCLLSIWRVLPTALLITL | 240 |
| NOV19: | 292 | FYTIVTPLLNPFIY | 305 |
| | | + \| \|\|\| \|\| | |
| Sbjct: | 241 | WLAYVNSCLNPIIY | 254 |

G-Protein Coupled Receptor (GPCRs) have been identified as extremely large subfamily of G protein-coupled receptors in a number of species. These receptors share a seven transmembrane domain structure with many neurotransmitter and hormone receptors, and are likely to underlie the recognition and G-protein-mediated transduction of various signals. Previously, GPCR genes cloned in different species were from random locations in the respective genomes. The human GPCR genes are intron less and belong to four different gene subfamilies, displaying great sequence variability. These genes are dominantly expressed in olfactory epithelium.

Olfactory receptors (ORs) have been identified as extremely large subfamily of G protein-coupled receptors in a number of species. These receptors share a seven transmembrane domain structure with many neurotransmitter and hormone receptors, and are likely to underlie the recognition and G-protein-mediated transduction of odorant signals. Previously, OR genes cloned in different species were from random locations in the respective genomes. The human OR genes are intron less and belong to four different gene subfamilies, displaying great sequence variability. These genes are dominantly expressed in olfactory epithelium.

The disclosed NOV19 nucleic acid of the invention encoding a G-Protein Coupled Receptor-like protein includes the nucleic acid whose sequence is provided in Table 19A, 19C or a fragment thereof. The invention also includes a mutant or variant nucleic acid any of whose bases may be changed from the corresponding base shown in Table 19A or 19C while still encoding a protein that maintains its G-Protein Coupled Receptor-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 35 percent of the bases may be so changed.

The disclosed NOV19 protein of the invention includes the G-Protein Coupled Receptor-like protein whose sequence is provided in Table 19B or 19D. The invention also includes a mutant or variant protein any of whose residues may be changed from the corresponding residue shown in Table 19B or 19D while still encoding a protein that maintains its G-Protein Coupled Receptor-like activities and physiological functions, or a functional fragment thereof. In the mutant or variant protein, up to about 52 percent of the residues may be so changed.

The invention further encompasses antibodies and antibody fragments, such as F_{ab} or (F_{ab})₂, that bind immunospecifically to any of the proteins of the invention.

The above disclosed information suggests that this G-Protein Coupled Receptor-like protein (NOV19) is a member of a “G-Protein Coupled Receptor family”. Therefore, the NOV19 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV19 nucleic acids and proteins of the invention are useful in potential therapeutic applications implicated in developmental diseases, MHCII and III diseases (immune diseases), Taste and scent detectability Disorders, Burkitt's lymphoma, Corticoneurogenic disease, Signal Transduction pathway disorders, Retinal diseases including those involving photoreception, Cell Growth rate disorders; Cell Shape disorders, Feeding disorders;control of feeding; potential obesity due to over-eating; potential disorders due to starvation (lack of apetite), noninsulin-dependent diabetes mellitus (NIDDM1), bacterial, fungal, protozoal and viral infections (particularly infections caused by HIV-1 or HIV-2), pain, cancer (including but not limited to Neoplasm; adenocarcinoma; lymphoma; prostate cancer; uterus cancer), anorexia, bulimia, asthma, Parkinson's disease, acute heart failure, hypotension, hypertension, urinary retention, osteoporosis, Crohn's disease; multiple sclerosis; and Treatment of Albright Hereditary Ostoeodystrophy, angina pectoris, myocardial infarction, ulcers, asthma, allergies, benign prostatic hypertrophy, and psychotic and neurological disorders, including anxiety, schizophrenia, manic depression, delirium, dementia, severe mental retardation. Dentatorubro-pallidoluysian atrophy(DRPLA) Hypophosphatemic rickets, autosomal dominant (2) Acrocallosal syndrome and dyskinesias, such as Huntington's disease or Gilles de la Tourette syndrome, and/or other diseases and pathologies.

NOV19 nucleic acids and polypeptides are further useful in the generation of antibodies that bind immuno-specifically to the novel NOV19 substances for use in therapeutic or diagnostic methods. These antibodies may be generated according to methods known in the art, using prediction from hydrophobicity charts, as described in the “Anti-NOVX Antibodies” section below. The disclosed NOV19 protein has multiple hydrophilic regions, each of which can be used as an immunogen. These novel proteins can be used in assay systems for functional analysis of various human disorders, which will help in understanding of pathology of the disease and development of new drug targets for various disorders.

NOV20

A disclosed NOV20 nucleic acid of 1027 nucleotides (also referred to as CG56665-01) encoding a novel G-Protein Coupled Receptor-like protein is shown in Table 20A. An open reading frame was identified beginning with an ATG initiation codon at nucleotides 1-3 and ending with a TAG codon at nucleotides 940-942. The start and stop codons are shown in bold in Table 20A, and the 5′ and 3′ untranslated regions, if any, are underlined.

TABLE 20A
NOV20 nucleotide sequence.

(SEQ ID NO:85)

ATGATCTGCTCAGCTATCAACCTACACTTACTACTGGCAGTTAAGATGATTCACCCTGTCTGGATTCTTGCT
CCTCGGGAGCAAGGGCTGTTTCTGCTGATTTATCTGGCAGTGCTGGTGGGGAACCTGCTCATCATTGCAGTC
ATCACTCTCGATCAGCATCTTCACACACCCATGTACTTCTTCCTGAAGAACCTCTCCGTTTTGGATCTGTGC
TACATCTCAGTCACTGTGCCTAAATCCATCCGTAACTCCCTGACTCGCAGAAGCTCCATCTCTTATCTTGGC
TGTGTGGCTCAAGTCTATTTTTTCTCTGCCTTTGCATCTGCTGAGCTGGCCTTCCTTACTGTCATGTCTTAT
GACCGCTATGTTGCCATTTGCCACCCCCTCCAATACAGAGCCGTGATGACATCAGGAGGGTGCTATCAGATG
GCAGTCACCACCTGGCTAAGCTCCTTTTCCTACGCAGCCGTCCACACTGGCAACATGTTTCGGGAGCACGTT
TGCAGATCCAGTGTGATCCACCAGTTCTTCCGTGACATCCCTCATGTGTTGGCCCTGGTTTCCTGTGAGGTT
TTCTTTGTAGAGTTTTTGACCCTGGCCCTGAGCTCATGCTTGGTTCTGGGATGCTTTATTCTCATGATGATC
TCCTATTTCCAAATCTTCTCAACGGTGCTCAGAATCCCTTCAGGACAGAGTCGAGCAAAAGCCTTCTCCACC
TGCTCCCCCCAGCTCATTGTCATCATGCTCTTTCTTACCACAGGGCTCTTTGCTGCCTTAGGACCAATTGCA
AAAGCTCTGTCCATTCAGGATTTAGTGATTGCTCTGACATACACAGTTTTGCCTCCCTTCCTCAATCCCATC
ATATATAGTCTTAGGAATAAGGAGATTAAAACAGCCATGTGGAGACTCTTTGTGAAGATATATTTTCTGCAA
AAGTAGAACATCCTGGTCTTTACTATAGAAGATCTGCAACAAAACCCCAAAAAAGCATAAATACTTTATGAC
AAAAAAAGATGAAAAAATT

The disclosed NOV20 polypeptide (SEQ ID NO:86) encoded by SEQ ID NO:85 has 313 amino acid residues and is presented in Table 20B using the one-letter amino acid code.

TABLE 20B
Encoded NOV20 protein sequence.

| | |
|---|---|
| MICSAINLHLLLAVKMIHPVWILAPREQGLFLLIYLAVLVGNLLIIAVITLDQHLHTPMYFFLK | (SEQ ID NO:86) |
| NLSVLDLCYISVTVPKSIRNSLTRRSSISYLGCVAQVYFFSAFASAELAFLTVMSYDRYVAICH | |
| PLQYRAVNTSGGCYQMAVTTWLSCFSYAAVHTGNMFREHVCRSSVIHQFFRDIPHVLALVSCEV | |
| FFVEFLTLALSSCLVLGCFILMMISYFQIFSTVLRIPSGQSRAKAFSTCSPQLIVIMLFLTTGL | |
| FAALGPIAKALSIQDLVIALTYTVLPPFLNPIIYSLRNKEIKTAMWRLFVKIYFLQK | |

A search of sequence databases reveals that the NOV20 amino acid sequence has 134 of 278 amino acid residues (48%) identical to, and 179 of 278 amino acid residues (64%) similar to, the 321 amino acid residue ptnr: SPTREMBL-ACC:Q9UGF5 BA150A6.4 protein from Homo sapiens (Human) (NOVEL 7 TRANSMEMBRANE RECEPTOR (RHODOPSIN FAMILY) (E=2.4e⁻⁶⁴).

The disclosed NOV20 polypeptide has homology to the amino acid sequences shown in the BLASTP data listed in Table 20C.

TABLE 20C
BLAST results for NOV20

| Gene Index/ | | Length | | Positives | |
|---|---|---|---|---|---|
| Identifier | Protein/Organism | (aa) | Identity (%) | (%) | Expect |
| gi\|17437075\|ref\|XP_{—} | similar to | 311 | 287/294 | 288/294 | e−134 |
| 060319.1\| | OLFACTORY | | (97%) | (97%) | |
| (XM_060319) | RECEPTOR 5U1 | | | | |
| | (HS6M1-28) (H. | | | | |
| | sapiens) [Homo | | | | |
| | sapiens] | | | | |
| gi\|17445373\|ref\|XP_{—} | similar to | 309 | 147/272 | 188/272 | 8e−63 |
| 060567.1\| | OLFACTORY | | (54%) | (69%) | |
| (XM_060567) | RECEPTOR 5U1 | | | | |
| | (HS6M1-28) (H. | | | | |
| | sapiens) [Homo | | | | |
| | sapiens] | | | | |
| gi\|17445394\|ref\|XP_{—} | similar to | 316 | 133/283 | 187/283 | 2e−61 |
| 060572.1\| | OLFACTORY | | (46%) | (65%) | |
| (XM_060572) | RECEPTOR 5U1 | | | | |
| | (HS6M1-28) (H. | | | | |
| | sapiens) [Homo | | | | |
| | sapiens] | | | | |
| gi\|17437015\|ref\|XP_{—} | similar to | 312 | 139/291 | 189/291 | 9e−59 |
| 060307.1\| | OLFACTORY | | (47%) | (64%) | |
| (XM_060307) | RECEPTOR 5U1 | | | | |
| | (HS6M1-28) (H. | | | | |
| | sapiens) [Homo | | | | |
| | sapiens] | | | | |
| gi\|17464351\|ref\|XP_{—} | similar to | 321 | 133/278 | 175/278 | 3e−57 |
| 069462.1\| | OLFACTORY | | (47%) | (62%) | |
| (XM_069462) | RECEPTOR 5U1 | | | | |
| | (HS6M1-28) (H. | | | | |
| | sapiens) [Homo | | | | |
| | sapiens] | | | | |

The homology between these and other sequences is shown graphically in the ClustalW analysis shown in Table 20D. In the ClustalW alignment of the NOV20 protein, as well as all other ClustalW analyses herein, the black outlined amino acid residues indicate regions of conserved sequence (i.e., regions that may be required to preserve structural or functional properties), whereas non-highlighted amino acid residues are less conserved and can potentially be altered to a much broader extent without altering protein structure or function.

[image]

[image]

Table 20E lists the domain descriptions from DOMAIN analysis results against NOV20. This indicates that the NOV20 sequence has properties similar to those of other proteins known to contain this domain.

TABLE 20E
Domain Analysis of NOV20

gnl|Pfam|pfam00001, 7tm_1, 7 transmembrane receptor (rhodopsin family)
CD-Length = 254 residues, 100.0% aligned
Score 83.6 bits (205), Expect = 2e−17

| | | | |
|---|---|---|---|
| NOV20: | 41 | GNLLIIAVITLDQHLHTPMYFFLKNLSVLDLCYISVTVPKSIRNSLTRRSSISYLGCVAQ | 100 |
| | | \|\|\|\|+\| \|\| + \| \|\| \|\| \|\|+\| \|\| ++ \| ++ + \| | |
| Sbjct: | 1 | GNLLVILVILRTKKLRTPTNIFLLNLAVADLLFLLTLPPWALYYLVGGDWVFGDALCKLV | 60 |
| NOV20: | 101 | VYFFSAFASAELAFLTVMSYDRYVAICHPLQYRAVMTSGGCYQMAVTTWLSCFSYAAVHT | 160 |
| | | \| \| + \|\| +\| \|\|\|+\|\| \|\|\|+\|\| + \| + + \|+ + | |
| Sbjct: | 61 | GALFVVNGYASILLLTAISIDRYLAIVHPLRYRRIRTPRRAKVLILLVWVLALLLSLPPL | 120 |
| NOV20: | 161 | GNMFREHVCRSSVIHQFFRDIPHVLALVSCEVFFVEFLTLALSSCLVLGCFILMMISYFQ | 220 |
| | | + \| + + + + \| \| \|\| \|\| + | |
| Sbjct: | 121 | LFSWLRTVEEGNTTVCLIDFPEESVKRSYVLLSTLVGFVLPLLVILVCYTRILRTLRKRA | 180 |
| NOV20: | 221 | IFSTVLRIPSGQSRAKAFSTCSPQLIVIMLFLTTGLFAALGPIAKALSIQDLVIALT--- | 277 |
| | | \|+ \| \| \| ++ ++ +\| + \| + + \| \|\| | |
| Sbjct: | 181 | RSQRSLKRRSSSERKAAKMLLVVVVVFVLCWLPYHIVLLLDSLCLLSIWRVLPTALLITL | 240 |
| NOV20: | 278 | -YTVLPPFLNPIIY | 290 |
| | | + \|\|\|\|\|\| | |
| Sbjct: | 241 | WLAYVNSCLNPIIY | 254 |

G-Protein Coupled Receptor (GPCRs) have been identified as extremely large subfamily of G protein-coupled receptors in a number of species. These receptors share a seven transmembrane domain structure with many neurotransmitter and hormone receptors, and are likely to underlie the recognition and G-protein-mediated transduction of various signals. Previously, GPCR genes cloned in different species were from random locations in the respective genomes. The human GPCR genes are intron less and belong to four different gene subfamilies, displaying great sequence variability. These genes are dominantly expressed in olfactory epithelium.

Olfactory receptors (ORs) have been identified as extremely large subfamily of G protein-coupled receptors in a number of species. These receptors share a seven transmembrane domain structure with many neurotransmitter and hormone receptors, and are likely to underlie the recognition and G-protein-mediated transduction of odorant signals. Previously, OR genes cloned in different species were from random locations in the respective genomes. The human OR genes are intron less and belong to four different gene subfamilies, displaying great sequence variability. These genes are dominantly expressed in olfactory epithelium.

The disclosed NOV20 nucleic acid of the invention encoding a G-Protein Coupled Receptor-like protein includes the nucleic acid whose sequence is provided in Table 20A or a fragment thereof. The invention also includes a mutant or variant nucleic acid any of whose bases may be changed from the corresponding base shown in Table 20A while still encoding a protein that maintains its G-Protein Coupled Receptor-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

The disclosed NOV20 protein of the invention includes the G-Protein Coupled Receptor-like protein whose sequence is provided in Table 20B. The invention also includes a mutant or variant protein any of whose residues may be changed from the corresponding residue shown in Table 20B while still encoding a protein that maintains its G-Protein Coupled Receptor-like activities and physiological functions, or a functional fragment thereof. In the mutant or variant protein, up to about 54 percent of the residues may be so changed.

The invention further encompasses antibodies and antibody fragments, such as F_{ab} or (F_{ab})₂, that bind immunospecifically to any of the proteins of the invention.

The above disclosed information suggests that this G-Protein Coupled Receptor-like protein (NOV20) is a member of a “G-Protein Coupled Receptor family”. Therefore, the NOV20 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV20 nucleic acids and proteins of the invention are useful in potential therapeutic applications implicated in developmental diseases, MHCII and III diseases (immune diseases), Taste and scent detectability Disorders, Burkitt's lymphoma, Corticoneurogenic disease, Signal Transduction pathway disorders, Retinal diseases including those involving photoreception, Cell Growth rate disorders; Cell Shape disorders, Feeding disorders;control of feeding; potential obesity due to over-eating; potential disorders due to starvation (lack of apetite), noninsulin-dependent diabetes mellitus (NIDDM1), bacterial, fungal, protozoal and viral infections (particularly infections caused by HIV-1 or HIV-2), pain, cancer (including but not limited to Neoplasm; adenocarcinoma; lymphoma; prostate cancer; uterus cancer), anorexia, bulimia, asthma, Parkinson's disease, acute heart failure, hypotension, hypertension, urinary retention, osteoporosis, Crohn's disease; multiple sclerosis; and Treatment of Albright Hereditary Ostoeodystrophy, angina pectoris, myocardial infarction, ulcers, asthma, allergies, benign prostatic hypertrophy, and psychotic and neurological disorders, including anxiety, schizophrenia, manic depression, delirium, dementia, severe mental retardation. Dentatorubro-pallidoluysian atrophy(DRPLA) Hypophosphatemic rickets, autosomal dominant (2) Acrocallosal syndrome and dyskinesias, such as Huntington's disease or Gilles de la Tourette syndrome, and/or other diseases and pathologies.

NOV20 nucleic acids and polypeptides are further useful in the generation of antibodies that bind immuno-specifically to the novel NOV20 substances for use in therapeutic or diagnostic methods. These antibodies may be generated according to methods known in the art, using prediction from hydrophobicity charts, as described in the “Anti-NOVX Antibodies” section below. The disclosed NOV20 protein has multiple hydrophilic regions, each of which can be used as an immunogen. These novel proteins can be used in assay systems for functional analysis of various human disorders, which will help in understanding of pathology of the disease and development of new drug targets for various disorders.

NOV21

NOV21 includes three novel adrenal secretory serine protease-like proteins disclosed below. The disclosed sequences have been named NOV21a and NOV21b.

NOV21a

A disclosed NOV21a nucleic acid of 1028 nucleotides (also referred to as CG56639-01) encoding a novel adrenal secretory serine protease-like protein is shown in Table 21A. An open reading frame was identified beginning with an TCG initiation codon at nucleotides 1-3 and ending with a TGA codon at nucleotides 769-771. The start and stop codons are shown in bold in Table 21A, and the 5′ and 3′ untranslated regions, if any, are underlined. Because the start codon of NOV21a is not a traditional initiation codon, NOV21a could be a partial reading frame that extends further in the 5′ direction.

TABLE 21A
NOV21a nucleotide sequence.

(SEQ ID NO:87)

TCGCCATTTCCAGACGCCCCGGAGGCCACCACACACACCCAGCTACCAGACTGTGGCCTGCCGCCGGCCGCG
CTCACCAGGATTGTGGGCGGCAGCGCAGCGGGCCGTGGGGAGTGGCCGTGGCAGGTGAGCCTGTGGCTGCGG
CGCCGGGAACACCGTTGCGGGGCCGTGCTGGTGGCAGAGAGGTGGCTGCTGTCGGCGGCGCACTGCTTCGAC
GTCTACGGGGACCCCAAGCAGTGGGCGGCCTTCCTAGGCACGCCGTTCCTGAGCGGCGCGGAGGGGCAGCTG
GAGCGCGTGGCGCGCATCTACAAGCACCCGTTCTACAATCTCTACACGCTCGACTACGACGTGGCGCTGCTG
GAGCTGGCGGGGCCGGTGCGTCGCAGCCGCCTGGTGCGTCCCATCTGCCTGCCCGAGCCCGCGCCGCGACCC
CCGGACGGCACGCGCTGCGTCATCACCGGCTGGGGCTCGGTGCGCGAAGGAGGCTCCATGGCGCGGCAGCTG
CAGAAGGCGGCCGTGCGCCTCCTCAGCGAGCAGACCTGCCGCCGCTTCTACCCAGTGCAGATCAGCAGCCGC
ATCTCTGAACCCCCTTTCTTCTCTCCCCAACAGGGTGACGCTGGGGGACCCCTGGCCTGCAGGGAGCCCTCT
GGACGGTGGCTGCTAACTGGGGTCACTAGCTGGGGCTATGGCTGTGGCCGGCCCCACTTCCCAGGTGTCTAT
ACCCGGGTGGCAGCTGTGAGAGGCTGGATAGGACAGCACATCCAGGAGTGACCACCACGTGACTGCCCAGGC
CGAGACTCTACGTGAAAGCAACAGGAGCAGCAGGCCACCCAACACCCCACCCCACCGTACCCTACCCAAGGA
CGGGTGTGGGGGGGCTGTGGGTCATGGGGATGCATTTTGGTACCACCCTTTGTTCCAATAAACACAGCCCCT
CCACCCTAGCTCACTGGCTCAGCACCTCAGTGTCACAGCGAGGACCACCTGCCTGGTGCTTCACCAGGACCC
GGGGTGGAACGAAACAACCC

In a search of public sequence databases, the NOV21a nucleic acid sequence, located on chromosome 19, has 296 of 466 bases (63%) identical to a gb:GENBANK-ID:E13204|acc:E13204.1 mRNA from Homo sapiens (Human cDNA encoding a serine protease) (E=3.9e⁻¹⁸).

The disclosed NOV21a polypeptide (SEQ ID NO:88) encoded by SEQ ID NO:87 has 256 amino acid residues and is presented in Table 21B using the one-letter amino acid code. Signal P, Psort and/or Hydropathy results predict that NOV21a has A signal peptide and is likely to be localized to the microbody (peroxisome) with a certainty of 0.7480. Alternatively, NOV21a may also localize to the lysosome (lumen) with a certainty of 0.3168, or the mitochondrial matrix space with a certainty of 0.1000. The most likely cleavage site for NOV21a is between positions 68 and 69: SAA-HC.

TABLE 21B
Encoded NOV21a protein sequence.

(SEQ ID NO:88)

SPFPDAPEATTHTQLPDCGLAPAALTRIVGGSAAGRGEWPWQVSLWLRRREHRCGAVLVAERWLLSAAHCFD
VYGDPKQWAAFLGTPFLSGAEGQLERVARIYKHPFYNLYTLDYDVALLELAGPVRRSRLVRPICLPEPAPRP
PDGTRCVITGWGSVREGGSMARQLQKAAVRLLSEQTCRRFYPVQISSRISEPPFFSPQQGDAGGPLACREPS
GRWVLTGVTSWGYGCGRPHFPGVYTRVAAVRGWIGQHIQE

A search of sequence databases reveals that the NOV21a amino acid sequence has 99 of 250 amino acid residues (39%) identical to, and 134 of 250 amino acid residues (53%) similar to, the 279 amino acid residue ptnr:SPTREMBL-ACC:Q9QZ74 protein from Rattus norvegicus (Rat) (Adrenal Secretory Serine Protease Precursor) (E=1.5e⁻⁴²).

NOV21a is predicted to be expressed in at least the following tissues: Ovary, kidney, breast, lung, muscle, liver, spleen, blood, lymphocyte. This information was derived by determining the tissue sources of the sequences that were included in the invention including but not limited to SeqCalling sources, Public EST sources, Literature sources, and/or RACE sources.

NOV21b

In the present invention, the target sequence identified previously, NOV21a, was subjected to the exon linking process to confirm the sequence. PCR primers were designed by starting at the most upstream sequence available, for the forward primer, and at the most downstream sequence available for the reverse primer. In each case, the sequence was examined, walking inward from the respective termini toward the coding sequence, until a suitable sequence that is either unique or highly selective was encountered, or, in the case of the reverse primer, until the stop codon was reached. Such primers were designed based on in silico predictions for the full length cDNA, part (one or more exons) of the DNA or protein sequence of the target sequence, or by translated homology of the predicted exons to closely related human sequences sequences from other species. These primers were then employed in PCR amplification based on the following pool of human cDNAs: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea, uterus. Usually the resulting amplicons were gel purified, cloned and sequenced to high redundancy. The resulting sequences from all clones were assembled with themselves, with other fragments in CuraGen Corporation's database and with public ESTs. Fragments and ESTs were included as components for an assembly when the extent of their identity with another component of the assembly was at least 95% over 50 bp. In addition, sequence traces were evaluated manually and edited for corrections if appropriate. These procedures provide the sequence reported below, which is designated Accession Number NOV21b. This differs from the previously identified sequence (NOV21a) in being a splice variant and a mature protein starting with serine.

A disclosed NOV21b nucleic acid of 785 nucleotides (also referred to as CG56639-02) encoding a novel adrenal secretory serine protease-like protein is shown in Table 21C. An open reading frame was identified beginning with an CTT initiation codon at nucleotides 1-3 and ending with a TGA codon at nucleotides 783-785. The start and stop codons are shown in bold in Table 21C, and the 5′ and 3′ untranslated regions, if any, are underlined. Because the start codon of NOV21b is not a traditional initiation codon, NOV21b could be a partial reading frame that extends further in the 5′ direction.

TABLE 21C
NOV21b nucleotide sequence.

(SEQ ID NO:89)

CTTCGCCATTTCCAGACGCCCCGGAGGCCACCACACACACCCAGCTACCAGACTGTGGCCTGGCGCCGGCCG
CGCTCACCAGGATTGTGGGCGGCAGCGCAGCGGGCCGTGGGGAGTGGCCGTGGCAGGTGAGCCTGTGGCTGC
GGCGCCGGGAACACCGTTGCGGGGCCGTGCTGGTGGCAGAGAGGTGGCTGCTGTCGGCGGCGCACTGCTTCG
ACGTCTACGGGGACCCCAAGCAGTGGGCGGCCTTCCTAGGCACGCCGTTCCTGAGCGGCGCGGAGGGGCAGC
TGGAGCGCGTGGCGCGCATCTACAAGCACCCGTTCTACAATCTCTACACGCTCGACTACGACGTGGCGCTGC
TGGAGCTGGCGGGGCCGGTGCGTCGCAGCCGCCTGGTGCGTCCCATCTGCCTGCCCGAGCCCGCGCCGCGAC
CCCCGGACGGCACGCGCTGCGTCATCACCGGCTGGGGCTCGGTGCGCGAAGGAGGCTCCATGGCGCGGCAGC
TGCAGAAGGCGGCGGTGCGCCTCCTCAGCGAGCAGACCTGCCACCGCTTCTACCCAGTGCAGATCAGCAGCC
GCATGCTGTGTGCCGGCTTCCCGCAGGGTGGCGTGGACAGCTGCTCGGGTGACGCTGGGGGACCCCTGCCCT
GCAGGGAGCCCTCTGGACGGTGGGTGCTAACTGGGGTCACTAGCTGGGGCTATGGCTGTGGCCGGCCCCACT
TCCCAGGTGTCTATACCCGGGTGGCAGCTGTGAGAGGCTGGATAGGACAGCACATCCAGGAGTGA

In a search of public sequence databases, the NOV21b nucleic acid sequence, located on chromosome 19, has 160 of 162 bases (98%) identical to a gb:GENBANK-ID:HUMLAMBBB|acc:M94363.1 mRNA from Homo sapiens (Human lamin B2 (LAMB2) gene and ppv1 gene sequence) (E=4.3e⁻⁵⁹).

The disclosed NOV21b polypeptide (SEQ ID NO:90) encoded by SEQ ID NO:89 has 260 amino acid residues and is presented in Table 21D using the one-letter amino acid code. Signal P, Psort and/or Hydropathy results predict that NOV21b has A signal peptide and is likely to be localized to the microbody (peroxisome) with a certainty of 0.7480. Alternatively, NOV21b may also localize to the lysosome (lumen) with a certainty of 0.3082, or the mitochondrial matrix space with a certainty of 0.1000. The most likely cleavage site for NOV21b is between positions 68 and 69: SAA-HC.

TABLE 21D
Encoded NOV21b protein sequence.

(SEQ ID NO:90)

SPFPDAPEATTHTQLPDCGLAPAALTRIVGGSAAGRGEWPWQVSLWLRRREHRCGAVLVAERWLLSAAHCFD
VYGDPKQWAAFLGTPFLSGAEGQLERVARIYKHPFYNLYTLDYDVALLELAGPVRRSRLVRPICLPEPAPRP
PDGTRCVITGWGSVREGGSMARQLQKAAVRLLSEQTCHRFYPVQISSRMLCAGFPQGGVDSCSGDAGGPLAC
REPSGRWVLTGVTSWGYGCGRPHFPGVYTRVAAVRGWIGQHIQE

A search of sequence databases reveals that the NOV21b amino acid sequence has 123 of 250 amino acid residues (49%) identical to, and 154 of 250 amino acid residues (61%) similar to, the 855 amino acid residue ptnr:SPTREMBL-ACC:Q9Y5Y6 protein from Homo sapiens (Human) (Matriptase) (E=3.5e⁻⁵⁹).

NOV21b is predicted to be expressed in at least the following tissues: adrenal gland, Ovary, kidney, breast, lung, muscle, liver, spleen, blood, lymphocyte.

The disclosed NOV21a polypeptide has homology to the amino acid sequences shown in the BLASTP data listed in Table 21E.

TABLE 21E
BLAST results for NOV21a

| Gene Index/ | | Length | Identity | Positives | |
|---|---|---|---|---|---|
| Identifier | Protein/ Organism | (aa) | (%) | (%) | Expect |
| gi\|12836503\|dbj\|BAB | data source:SPTR, | 799 | 118/244 | 153/244 | 7e−55 |
| 23684.1\| (AK004939) | source | | (48%) | (62%) | |
| | key:O95519, | | | | |
| | evidence: ISS˜homo | | | | |
| | log to DJ1170K4.4 | | | | |
| | (NOVEL PROTEIN) | | | | |
| | (FRAGMENT)˜putative | | | | |
| | [Mus musculus] | | | | |
| gi\|10257390\|gb\|AAG1 | serine protease | 855 | 115/250 | 146/250 | 6e−52 |
| 5395.1\|AF057145_1 | TADG15 [Homo | | (46%) | (58%) | |
| (AF057145) | sapiens] | | | | |
| gi\|11415040\|ref\|NP_{—} | suppression of | 855 | 115/250 | 146/250 | 7e−52 |
| 068813.1\| | tumorigenicity 14 | | (46%) | (58%) | |
| (NM_021978) | (colon carcinoma, | | | | |
| | matriptase, | | | | |
| | epithin); | | | | |
| | suppression of | | | | |
| | tumorigenicity 14 | | | | |
| | (colon | | | | |
| | carcinoma); | | | | |
| | matriptase [Homo | | | | |
| | sapiens] | | | | |
| gi\|7363445\|ref\|NP_{—} | protease, serine, | 855 | 115/250 | 144/250 | 8e−52 |
| 035306.2\| | 14 (epithin) [MUS | | (46%) | (57%) | |
| (NM_011176) | musculus] | | | | |
| gi\|16758444\|ref\|NP_{—} | suppression of | 855 | 112/247 | 141/247 | 1e−51 |
| 446067.1\| | tumorigenicity 14 | | (45%) | (56%) | |
| (NM_053635) | (colon carcinoma, | | | | |
| | matriptase, | | | | |
| | epithin) [Rattus | | | | |
| | norvegicus] | | | | |

The homology between these and other sequences is shown graphically in the ClustalW analysis shown in Table 21F. In the ClustalW alignment of the NOV21 protein, as well as all other ClustalW analyses herein, the black outlined amino acid residues indicate regions of conserved sequence (i.e., regions that may be required to preserve structural or functional properties), whereas non-highlighted amino acid residues are less conserved and can potentially be altered to a much broader extent without altering protein structure or function.

[image]

[image]

[image]

Tables 21G-H lists the domain descriptions from DOMAIN analysis results against NOV21. This indicates that the NOV21 sequence has properties similar to those of other proteins known to contain this domain.

TABLE 21G
Domain Analysis of NOV21

gnl|Smart|smart00020, Tryp_SPc, Trypsin-like serine protease; Many of
these are synthesised as inactive precursor zymogens that are cleaved
during limited proteolysis to generate their active forms. A few,
however, are active as single chain molecules, and others are inactive
due to substitutions of the catalytic triad residues. (SEQ ID NO:812)
CD-Length = 230 residues, 100.0% aligned
Score = 221 hits (563), Expect = 4e−59

| | | | |
|---|---|---|---|
| NOV21: | 27 | RIVGGSAAGRGEWPWQVSLWLRRREHRCGAVLVAERWLLSAAHCFDVYGDPKQWAAFLGT | 86 |
| | | \|\|\|\|\|\| \| \| +\|\|\|\|\|\| \| \| \|\| \|++ \|\|+\|+\|\|\|\| \| \|\|+ | |
| Sbjct: | 1 | RIVGGSEANIGSFPWQVSLQYRCGGRHFCCGSLISPRWVLTAAHCVY-GSAPSSIRVRLGS | 59 |
| NOV21: | 87 | PFL-SGAEGQLERVARIYKHPFYNLYTLDYDVALLELAGPVRRSRLVRPICLPEPAPRPP | 145 |
| | | \| \|\| \| \| +\|+++ \|\| \|\| \| \| \|+\|\|\|+\|+ \|\| \| \|\|\|\|\|\|\| \| | |
| Sbjct: | 60 | HDLSSGEETQTVKVSKVIVHPNYNPSTYDNDIALLKLSEPVTLSDTVRPICLPSSGYNVP | 119 |
| NOV21: | 146 | DGTRCVITGWGSVRE-GGSMARQLQKAAVRLLSEQTCRRFYPVQISSRISEPPFFSPQ-- | 202 |
| | | \|\| \| ++\|\|\| \| \|\|+ \|\|+ \| ++\| \|\|\|\| \| + + + | |
| Sbjct: | 120 | AGTTCTVSGWGRTSESSGSLPDTLQEVNVPIVSNATCRRAYSGGPAITDNMLCAGGLEGG | 179 |
| NOV21: | 203 | ----QGDAGGPLACREPSGRWVLTGVTSWG-YGCGRPHFPGVYTRVAAVRGWI | 250 |
| | | \|\|\|+\|\|\|\| \|+ \|\|\| \|\|\| \|\|+ \|\|\|\|\|\|\|++ \|\| | |
| Sbjct: | 180 | KDACQGDSGGPLVCN--DPRWVLVGIVSWGSYGCARPNKPGVYTRVSSYLDWI | 230 |

TABLE 21H
Domain Analysis of NOV21

gnl|Pfam|pfam00089, trypsin, Trypsin. Proteins recognized include all
proteins in families S1, S2A, S2B, S2C, and S5 in the classification
of peptidases. Also included are proteins that are clearly members,
but that lack peptidase activity, such as haptoglobin and protein Z
(PRTZ*). (SEQ ID NO:813)
CD-Length = 217 residues, 100.0% aligned
Score = 177 bits (448), Expect = 9e−46

| | | | |
|---|---|---|---|
| NOV21: | 28 | IVGGSAAGRGEWPWQVSLWLRRREHRCGAVLVAERWLLSAAHCFDVYGDPKQWAAFLGTP | 87 |
| | | \|\|\|\| \| \| +\|\|\|\|\|\| + \| \|\| \|++\| \|+\|+\|\|\|\| \|\| | |
| Sbjct: | 1 | IVGGREAQAGSFPWQVSLQVSSG-HFCGGSLISENWVLTAAHCVS---GASSVRVVLGEH | 56 |
| NOV21: | 88 | FLSGAEGQLER--VARIYKHPFYNLYTLDYDVALLELAGPVRRSRLVRPICLPEPAPRPP | 145 |
| | | \| \|\| ++ \| +\| \|\| \|\| \| \|+\|\|\|+\| \|\| \|\|\|\|\|\|\| + \| | |
| Sbjct: | 57 | NLGTTEGTEQKFDVKKIIVHPNYNPDT--NDIALLKLKSPVTLGDTVRPICLPSASSDLP | 114 |
| NOV21: | 146 | DGTRCVITGWGSVREGGSMARQLQKAAVRLLSEQTCRRFYPVQISSR---ISEPPFFSPQ | 202 |
| | | \|\| \| ++\|\|\| + \|+ + \|\|+ \| ++\| +\|\|\| \| ++ | |
| Sbjct: | 115 | VGTTCSVSGWGRTKNLGT-SDTLQEVVVPIVSRETCRSAYGGTVTDTMICAGALGGKDAC | 173 |
| NOV21: | 203 | QGDAGGPLACREPSGRWVLTGVTSWGYGCGRPHFPGVYTRVAAVRGWI | 250 |
| | | \|\|\|+\|\|\|\| \| + \| \|+ \|\|\|\|\|\| \|\|\|\|\|\|\|+ \|\| | |
| Sbjct: | 174 | QGDSGGPLVCSDG----ELVGIVSWGYGCAVGNYPGVYTRVSRYLDWI | 217 |

Proteolytic enzymes that exploit serine in their catalytic activity are ubiquitous, being found in viruses, bacteria and eukaryotes. They include a wide range of peptidase activity, including exopeptidase, endopeptidase, oligopeptidase and omega-peptidase activity. Over 20 families (denoted S1-S27) of serine protease have been identified, these being grouped into 6 clans on the basis of structural similarity and other functional evidence.

Tryptase is a tetrameric serine protease that is concentrated and stored selectively in the secretory granules of all types of mast cells, from which it is secreted during mast cell degranulation. Its exclusive presence in mast cells permits its use as a specific clinical indicator of mast cell activation by measurement of its level in biologic fluids and as a selective marker of intact mast cells using immunohistochemical techniques with antitryptase antibodies.

In addition, NOV21 nucleic acids and polypeptides are useful, inter alia, as novel members of the protein families according to the presence of domains and sequences related to previously described proteins. For example, NOV21 nucleic acids and polypeptides contain a structural motif that is characteristic of protein sbelonging to the serine protease family of proteins. Accordingly, NOV21 may be useful in the same ways other members of this family are useful as detailed above.

The disclosed NOV21 nucleic acid of the invention encoding a Adrenal secretory serine protease-like protein includes the nucleic acid whose sequence is provided in Table 21A, 21C or a fragment thereof. The invention also includes a mutant or variant nucleic acid any of whose bases may be changed from the corresponding base shown in Table 21A or 21C while still encoding a protein that maintains its Adrenal secretory serine protease-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 2 percent of the bases may be so changed.

The disclosed NOV21 protein of the invention includes the Adrenal secretory serine protease-like protein whose sequence is provided in Table 21B or 24D. The invention also includes a mutant or variant protein any of whose residues may be changed from the corresponding residue shown in Table 21B or 21D while still encoding a protein that maintains its Adrenal secretory serine protease-like activities and physiological functions, or a functional fragment thereof. In the mutant or variant protein, up to about 54 percent of the residues may be so changed.

The invention further encompasses antibodies and antibody fragments, such as F_{ab} or (Fab)₂, that bind immunospecifically to any of the proteins of the invention.

The above disclosed information suggests that this Adrenal secretory serine protease-like protein (NOV21) is a member of a “Adrenal secretory serine protease family”. Therefore, the NOV21 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV21 nucleic acids and proteins of the invention are useful in potential therapeutic applications implicated in Von Hippel-Lindau (VHL) syndrome, cirrhosis, transplantation, endometriosis, fertility, anemia, ataxia-telangiectasia, autoimmune disease, hemophilia, hypercoagulation, idiopathic thrombocytopenic purpura, allergies, immunodeficiencies, graft versus host disease (GVHD), lymphaedema, muscular dystrophy, Lesch-Nyhan syndrome, myasthenia gravis, and/or other diseases and pathologies.

NOV21 nucleic acids and polypeptides are further useful in the generation of antibodies that bind immuno-specifically to the novel NOV21 substances for use in therapeutic or diagnostic methods. These antibodies may be generated according to methods known in the art, using prediction from hydrophobicity charts, as described in the “Anti-NOVX Antibodies” section below. The disclosed NOV21 protein has multiple hydrophilic regions, each of which can be used as an immunogen. These novel proteins can be used in assay systems for functional analysis of various human disorders, which will help in understanding of pathology of the disease and development of new drug targets for various disorders.

NOV22

NOV22 includes three novel adrenal secretory serine protease-like proteins disclosed below. The disclosed sequences have been named NOV22a, and NOV22b.

NOV22a

A disclosed NOV22a nucleic acid of 796 nucleotides (also referred to as CG56643-01) encoding a novel adrenal secretory serine protease-like protein is shown in Table 22A. An open reading frame was identified beginning with an ACC initiation codon at nucleotides 1-3 and ending with a TGA codon at nucleotides 763-765. The start and stop codons are shown in bold in Table 22A, and the 5′ and 3′ untranslated regions, if any, are underlined. Because the start codon of NOV22a is not a traditional initiation codon, NOV22a could be a partial reading frame that extends further in the 5′ direction.

TABLE 22A
NOV22a nucleotide sequence.

(SEQ ID NO:91)

ACCCGAGCAGGCCAAGATCCCCAGACCTGGTCTTGTGTCCTCCTTCCAGAATGTGGGGCCAGGCCTGCAATG
GAGAAGCCCACCCGGGTCGTGCGCGGGTTCGGAGCTGCCTCCGGGGAGGTGCCCTGGCAGGTCAGCCTGAAG
GAAGGGTCCCGGCACTTCTGCGGAGCAACTGTGGTGGGGGACCGCTGGCTGCTGTCTGCCGCCCACTGCTTC
CATAGCACGAAGGTGGAGCAGGTTCGGGCCCACCTGGGCACTGCGTCCCTCCTGGGCCTGGGCGGGAGCCCG
GTGAAGATCGGGCTGCGGCGGGTAGTGCTGCACCCCCTCTACAACCCTGGCATCCTGGACTTCGACCTGGCT
GTCCTGGAGCTGGCCAGCCCCCTGGCCTTCAACAAATACATCCAGCCTGTCTGCCTGCCCCTGGCCATCCAG
AAGTTCCCTGTGGGCCGGAAGTGCATGATCTCCGGATGGGGAAATACGCAGGAAGGAAATCTGCAGAAGGCG
TCCGTGGGCATCATAGACCAGAAAACCTGTAGTGTGCTCTACAACTTCTCCCTCACAGACCGCATGATCTGC
GCAGGCTTCCTGGAAGGCAAAGTCGACTCCTGCCAGGGTGACTCTGGGGGCCCCCTGGCCTGCGAGGAGGCC
CCTGGCGTGTTTTATCTGGCAGGGATCGTGAGCTGGGGTATTGGCTGCGCTCAGGTTAAGAAGCCGGGCGTG
TACACGCGCATCACCAGGCTAAAGGGCTGGATCATCCAGGAGTGACCACCACGTGACTGCCCAGGCCGAGAC
TCTA

In a search of public sequence databases, the NOV22a nucleic acid sequence, located on chromosome 19, has 278 of 428 bases (64%) identical to a gb:GENBANK-ID:E13204|acc:E13204.1 mRNA from Homo sapiens (Human cDNA encoding a serine protease) (E=1.6e⁻²⁹).

The disclosed NOV22a polypeptide (SEQ ID NO:92) encoded by SEQ ID NO:91 has 254 amino acid residues and is presented in Table 22B using the one-letter amino acid code. Signal P, Psort and/or Hydropathy results predict that NOV22a has no signal peptide and is likely to be localized to the microbody (peroxisome) with a certainty of 0.5090. Alternatively, NOV22a may also localize to the cytoplasm with a certainty of 0.4500, to the lysosome (lumen) with a certainty of 0.2082, or the mitochondrial matrix space with a certainty of 0.1000.

TABLE 22B
Encoded NOV22a protein sequence.

(SEQ ID NO:92)

TRAGQDPQTWSCVLLPECGARPAMEKPTRVVRGFGAASGEVPWQVSLKEGSRHFCGATVVGDRWLLSAAHCF
HSTKVEQVRAHLGTASLLGLGGSPVKIGLRRVVLHPLYNPGILDFDLAVLELASPLAFNKYIQPVCLPLAIQ
KFPVGRKCMISGWGNTQEGNLQKASVGIIDQKTCSVLYNFSLTDRMICAGFLEGKVDSCQGDSGGPLACEEA
PGVFYLAGIVSWGIGCAQVKKPGVYTRITRLKGWIIQE

A search of sequence databases reveals that the NOV22a amino acid sequence has 100 of 241 amino acid residues (41%) identical to, and 149 of 241 amino acid residues (61%) similar to, the 273 amino acid residue ptnr:TREMBLNEW-ACC:BAB20278 protein from Mus musculus (Mouse) (Type I Spinesin) (E=3.1e⁻⁴⁹).

The adrenal secretory serine protease disclosed in this invention is predicted to be expressed in at least the following tissues: Ovary, kidney, breast, lung, muscle, liver, spleen, blood, lymphocyte. This information was derived by determining the tissue sources of the sequences that were included in the invention including but not limited to SeqCalling sources, Public EST sources, Literature sources, and/or RACE sources.

NOV22b

In the present invention, the target sequence identified previously, NOV22a, was subjected to the exon linking process to confirm the sequence. PCR primers were designed by starting at the most upstream sequence available, for the forward primer, and at the most downstream sequence available for the reverse primer. In each case, the sequence was examined, walking inward from the respective termini toward the coding sequence, until a suitable sequence that is either unique or highly selective was encountered, or, in the case of the reverse primer, until the stop codon was reached. Such primers were designed based on in silico predictions for the full length cDNA, part (one or more exons) of the DNA or protein sequence of the target sequence, or by translated homology of the predicted exons to closely related human sequences sequences from other species. These primers were then employed in PCR amplification based on the following pool of human cDNAs: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea, uterus. Usually the resulting amplicons were gel purified, cloned and sequenced to high redundancy. The resulting sequences from all clones were assembled with themselves, with other fragments in CuraGen Corporation's database and with public ESTs. Fragments and ESTs were included as components for an assembly when the extent of their identity with another component of the assembly was at least 95% over 50 bp. In addition, sequence traces were evaluated manually and edited for corrections if appropriate. These procedures provide the sequence reported below, which is designated NOV22b. This differs from the previously identified sequence (NOV22a) in having 43 additional aminoacids and different N and C terminus.

A disclosed NOV22b nucleic acid of 992 nucleotides (also referred to as CG56643-02) encoding a novel adrenal secretory serine protease-like protein is shown in Table 22C. An open reading frame was identified beginning with an ATG initiation codon at nucleotides 101-103 and ending with a TAA codon at nucleotides 920-922. The start and stop codons are shown in bold in Table 22C, and the 5′ and 3′ untranslated regions, if any, are underlined.

TABLE 22C
NOV22b nucleotide sequence.

(SEQ ID NO:93)

GCTAGTCTATCCCGAGACCCCTCCCACTCCAACAGTTAATGCTTCCCTTGACCTCAGAATGGCCTCCTACAC
CTTACCCAGGTGCTAGGGCGGCAGCCCCATGGGGACAGTGGGGAGACTCTTGCGCTCTGAGCGGGCCATCAG
GCCCACCTCCTCCTCACTCTGTGGCTTTGTGAGATTCCTGCAACTCTGTGAGCCCTGGTTTCTTCGTCTGTG
GGGTGGGGATGCTGCATCTCGGGGCTGTTATCGGAGCGGAACTGGAGCTGCTCTGATGATCACTGTGCACGT
GGCCTTTCTGGCTCTTTCCCTGGTAGCCACCAAGCCCGAGCTCCTGCAGAAGGCGTCCGTGGGCATCATAGA
CCAGAAAACCTGTAGTGTGCTCTACAACTTCTCCCTCACAGACCGCATGATCTGCGCAGGCTTCCTGGAAGG
CAAAGTCGACTCCTGCCAGGGTGACTCTGGGGGCCCCCTGGCCTGCGAGGAGGCCCCTGGCGTGTTTTATCT
GGCAGGGATCGTGAGCTGGGGTATTGGCTGCGCTCAGGTTAAGAAGCCGGGCGTGTACACGCGCATCACCAG
GCTAAAGGGCTGGATCCTGGAGATCATGTCCTCCCAGCCCCTTCCCATGTCTCCCCCCTCGACCACAAGGAT
GCTGGCCACCACCAGCCCCAGGACGACAGCTGGCCTCACAGTCCCGGGGGCCACACCCAGCAGACCCACCCC
TGGGGCTGCCAGCAGGGTGACGGGCCAACCTGCCAACTCAACCTTATCTGCCGTGAGCACCACTGCTAGGGG
ACAGACGCCATTTCCAGACGCCCCGGAGGCCACCACACACACCCAGCTACCAGGTACCGGGAGAGACGGAGG
GATCCCTGGGAGTCGAGGGTCCCATGTTAATCAGCCTGGGCTGCCTAACAAGACATAACGTCGTCCACTTTG
GGAGGCCGAGGCGGGCGGATCAAGAGGTCAGGAGATCGAGACCATCCTGGCGAACA

In a search of public sequence databases, the NOV22b nucleic acid sequence, located on chromosome 19, has 203 of 294 bases (69%) identical to a gb:GENBANK-ID:AF133086|acc:AF133086.1 mRNA from Homo sapiens (membrane-type serine protease 1 mRNA, complete cds) (E=3.6e⁻¹⁶).

The disclosed NOV22b polypeptide (SEQ ID NO:94) encoded by SEQ ID NO:93 has 273 amino acid residues and is presented in Table 22D using the one-letter amino acid code. Signal P, Psort and/or Hydropathy results predict that NOV22b has A signal peptide and is likely to be localized to the mitochondrial inner membrane with a certainty of 0.8723. Alternatively, NOV22b may also localize to the plasma membrane with a certainty of 0.6500, to the mitochondrial intermembrane space with a certainty of 0.5053, or the mitochondrial matrix space with a certainty of 0.3617. The most likely cleavage site for NOV22b is between positions 43 and 44: GDA-AS.

TABLE 22D
Encoded NOV22b protein sequence.

(SEQ ID NO:94)

MGTVGRLLRSERAIRPTSSSLCGFVRFLQLCEPWFLRLWGGDAASRGCYRSGTGAALMITVHVAFLALSLVA
TKPELLQKASVGIIDQKTCSVLYNFSLTDRMICAGFLEGKVDSCQGDSGGPLACEEAPGVFYLAGIVSWGIG
CAQVKKPGVYTRITRLKGWILEIMSSQPLPMSPPSTTRMLATTSPRTTAGLTVPGATPSRPTPGAASRVTGQ
PANSTLSAVSTTARGQTPFPDAPEATTHTQLPGTGRDGGIPGSGGSHVNQPGLPNKT

A search of sequence databases reveals that the NOV22b amino acid sequence has 49 of 90 amino acid residues (54%) identical to, and 63 of 90 amino acid residues (70%) similar to, the 277 amino acid residue ptnr:SPTREMBL-ACC:O96899 protein from Scolopendra subspinipes (Plasminogen Activator Spa) (E=4.3e⁻²⁴).

NOV22b is predicted to be expressed in at least the following tissues: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea and uterus.

NOV22c

A disclosed NOV22c nucleic acid of 912 nucleotides (also referred to as CG56643-03) encoding a novel adrenal secretory serine protease-like protein is shown in Table 22E. An open reading frame was identified beginning with an ATG initiation codon at nucleotides 77-79 and ending with a TAA codon at nucleotides 896-898. The start and stop codons are shown in bold in Table 22E, and the 5′ and 3′ untranslated regions, if any, are underlined.

TABLE 22E
NOV22c nucleotide sequence.

(SEQ ID NO:95)

SCACTCCAACACTTAATGCTTCCCTTGACCTCAGAATGGCCTCCTACACCTTACCCAGGTGCTAGGGCGGCAG
CCCCATGGGGACAGTGGGGAGACTCTTGCGCTCTGAGCGCGCCATCAGGCCCACCTCCTCCTCACTCTGTGG
CTTTGTGAGATTCCTGCAACTCTGTGAGCCCTCGTTTCTTCGTCTGTGGGGTGGGGATGCTGCATCTCGGGG
CTGTTATCGGAGCGGAACTGGAGCTCCTCTGATGATCACTGTGCACGTGGCCTTTCTGCCTCTTTCCCTGGT
AGCCACCAAGCCCGAGCTCCTGCAGAAGGCGTCCGTGGGCATCATAGACCAGAAAACCTGTAGTGTGCTCTA
CAACTTCTCCCTCACAGACCGCATGATCTGCGCAGGCTTCCTGGAAGGCAAAGTCGACTCCTGCCAGGGTGA
CTCTGGGGGCCCCCTGGCCTGCGAGGAGGCCCCTGGCGTGTTTTATCTGGCAGGGATCGTGAGCTGGGGTAT
TGCCTGCGCTCAGGTTAACAAGCCGGGCGTGTACACGCGCATCACCAGGCTAAAGGGCTGGATCCTGGAGAT
CATGTCCTCCCAGCCCCTTCCCATGTCTCCCCCCTCGACCACAAGGATGCTGGCCACCACCAGCCCCAGGAC
GACAGCTGGCCTCACAGTCCCGGGGGCCACACCCAGCAGACCCACCCCTGGGGCTGCCAGCAGGGTGACGGG
CCAACCTGCCAACTCAACCTTATCTGCCGTGAGCACCACTGCTAGGGGACAGACGCCATTTCCACACGCCCC
CGAGGCCACCACACACACCCAGCTACCAGGTACCGGGAGAGACGGAGGGATCCCTGGGAGTGGAGGGTCCCA
TGTTAATCAGCCTGGGCTGCCTAACAAGACATAACGTCGTCCACTTTG

In a search of public sequence databases, the NOV22c nucleic acid sequence, located on chromosome 19, has 203 of 294 bases (69%) identical to a gb:GENBANK-ID:E13204|acc:E13204.1 mRNA from Homo sapiens (Human cDNA encoding a serine protease) (E=1.3e⁻¹⁸).

The disclosed NOV22c polypeptide (SEQ ID NO:96) encoded by SEQ ID NO:95 has 273 amino acid residues and is presented in Table 22F using the one-letter amino acid code. Signal P, Psort and/or Hydropathy results predict that NOV22c has A signal peptide and is likely to be localized to the mitochondrial inner membrane with a certainty of 0.8723. Alternatively, NOV22c may also localize to the plasma membrane with a certainty of 0.6500, to the mitochondrial intermembrane space with a certainty of 0.5053, or the mitochondrial matrix space with a certainty of 0.3617. The most likely cleavage site for NOV22c is between positions 43 and 44: GDA-AS.

TABLE 22F
Encoded NOV22c protein sequence.

(SEQ ID NO:96)

MGTVGRLLRSERAIRPTSSSLCGFVRFLQLCEPWFLRLWGGDAASRGCYRSGTGAALMITVHVAFLALSLVA
TKPELLQKASVGIIDQKTCSVLYNFSLTDRMICAGFLEGKVDSCQGDSGGPLACEEAPGVFYLAGIVSWGIG
CAQVKKPGVYTRITRLKGWILEIMSSQPLPMSPPSTTRMLATTSPRTTAGLTVPGATPSRPTPGAASRVTGQ
PANSTLSAVSTTARGQTPFPDAPEATTHTQLPGTGRDGGIPGSGGSHVNQPGLPNKT

A search of sequence databases reveals that the NOV22c amino acid sequence has 49 of 90 amino acid residues (54%) identical to, and 63 of 90 amino acid residues (70%) similar to, the 277 amino acid residue ptnr:SPTREMBL-ACC:O96899 protein from Scolopendra subspinipes (Plasminogen Activator SPA) (E=4.5e⁻²⁴).

NOV22c is predicted to be expressed in at least the following tissues: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea and uterus.

The disclosed NOV22a polypeptide has homology to the amino acid sequences shown in the BLASTP data listed in Table 22G.

TABLE 22G
BLAST results for NOV22a

| Gene Index/ | | Length | Identity | Positives | |
|---|---|---|---|---|---|
| Identifier | Protein/Organism | (aa) | (%) | (%) | Expect |
| gi\|16758444\|ref\|NP_{—} | suppression of | 855 | 109/251 | 148/251 | 7e−55 |
| 446087.1\| | tumorigenicity 14 | | (43%) | (58%) | |
| (NM_053635) | (colon carcinoma, | | | | |
| | matriptase, | | | | |
| | epithin) [Rattus | | | | |
| | norvegicus] | | | | |
| gi\|7363445\|ref\|NP_035306.2\| | protease, serine, | 855 | 110/248 | 150/248 | 7e−54 |
| (NM_011176) | 14 (epithin) [Mus | | (44%) | (60%) | |
| | musculus] | | | | |
| gi\|9757702\|dbj\|BAB08218.1\| | homolog of human | 845 | 113/261 | 156/261 | 2e−52 |
| (AB038498) | MT-SP1 [Xenopus | | (43%) | (59%) | |
| | laevis] | | | | |
| gi\|10257390\|gb\|AAG15395.1\| | serine protease | 855 | 107/248 | 145/248 | 3e−52 |
| AF057145_1 | TADG15 [Homo | | (43%) | (58%) | |
| (AF057145) | sapiens] | | | | |
| gi\|11415040\|ref\|NP_{—} | suppression of | 855 | 107/248 | 145/248 | 3e−52 |
| 068813.1\| | tumorigenicity 14 | | (43%) | (58%) | |
| (NM_021978) | (colon carcinoma, | | | | |
| | matriptase, | | | | |
| | epithin); | | | | |
| | suppression of | | | | |
| | tumorigenicity 14 | | | | |
| | (colon | | | | |
| | carcinoma); | | | | |
| | matriptase [Homo | | | | |
| | sapiens] | | | | |

The homology between these and other sequences is shown graphically in the ClustalW analysis shown in Table 22H. In the ClustalW alignment of the NOV22 protein, as well as all other ClustalW analyses herein, the black outlined amino acid residues indicate regions of conserved sequence (i.e., regions that may be required to preserve structural or functional properties), whereas non-highlighted amino acid residues are less conserved and can potentially be altered to a much broader extent without altering protein structure or function.

[image]

[image]

[image]

[image]

Tables 22I-J lists the domain descriptions from DOMAIN analysis results against NOV22. This indicates that the NOV22 sequence has properties similar to those of other proteins known to contain this domain.

TABLE 22I
Domain Analysis of NOV22

gnl|Smart|smart00020, Tryp_SPc, Trypsin-like serine protease; Many of
these are synthesised as inactive precursor zytnogens that are cleaved
during limited proteolysis to generate their active forms. A few,
however, are active as single chain molecules, and others are inactive
due to substitutions of the catalytic triad residues. (SEQ ID
NO: 812)
CD-Length = 230 residues, 100.0% aligned
Score = 220 bits (560), Expect 9e−59

| | | | |
|---|---|---|---|
| NOV22: | 29 | RVVRGFGAASGEVPWQVSLK-EGSRHFCGATVVGDRWLLSAAHCFHSTKVEQVRAHLGTA | 87 |
| | | \|+\|\| \| \| \|\|\|\|\|\|+ \|\|\|\|\|\|+++ \|\|+\|+\|\|\|\|++ +\| \|\|+ | |
| Sbjct: | 1 | RIVGGSEANIGSFPWQVSLQYRGCRHFCGGSLISPRWVLTAAHCVYOSAPSSIRVRLGSH | 60 |
| NOV22: | 88 | SLLGLGGSPVKIGLRRVVLHPLYNPGILDFDLAVLELASPLAFNKYIQPVCLPLAIQKFP | 147 |
| | | \| +++\|++\|\|\|\|\| \|\|+\|+\|+\|+\|+ + ++\|+\|\|\|+ \| | |
| Sbjct: | 61 | DLSSGEE-TQTVKVSKVIVHPNYNPSTYDNDIALLKLSEPVTLSDTVRPICLPSSGYNVP | 119 |
| NOV22: | 148 | VGRKCMISGWGNTQEGN------LQKASVGIIDQKTCSVLY--NFSLTDRMICAGFLEGK | 199 |
| | | \| \|+\|\|\|\|\|1 + \|\|++\|\|+ \|\| \| ++\|\|\|+\|\|\|\|\|\| | |
| Sbjct: | 120 | AGTTCTVSGWGRTSESSGSLPDTLQEVNVPIVSNATCRRAYSGGPAITDNMLCAGGLEGG | 179 |
| NOV22: | 200 | VDSCQGDSGGPLACEEAPGVFYLAGIVSWG-IGCAQVKKPGVYTRITRLKGWI | 251 |
| | | \|+\|\|\|\|\|\|\|\|\|\|+ +\|\|\|\|\|\|\|\| \|\|\|+ \|\|\|\|\|\|\|++ \|\| | |
| Sbjct: | 180 | KDACQGDSGGPLVCND--PRWVLVGIVSWGSYGCARPNKPGVYTRVSSYLDWI | 230 |

TABLE 22J
Domain Analysis of NOV22

gnl|Pfam|pfam00089, trypsin, Trypsin. Proteins recognized include all
proteins in families S1, S2A, S2B, S2C, and S5 in the classification
of peptidases. Also included are proteins that are clearly members,
but that lack peptidase activity, such as haptoglobin and protein Z
(PRTZ*). (SEQ ID NO:813)
CD-Length = 217 residues, 100.0% aligned
Score = 192 bits (488), Expect = 2e−50

| | | | |
|---|---|---|---|
| NOV22: | 30 | VVRGFGAASGEVPWQVSLKEGSRHFCGATVVGDRWLLSAAHCFHSTKVEQVRAHLGTASL | 89 |
| | | +\|\| \|+\| \|\|\|\|\|\|+ \|\|\|\|\|++++\|+\|+\|\|\|\| +\| \| +\| | |
| Sbjct: | 1 | IVGGREAQAGSFPWQVSLQVSSGHFCGGSLISENWVLTAAHCVSGASSVRVVL--GEHNL | 58 |
| NOV22: | 90 | LGLGGSPVKIGLRRVVLHPLYNPGILDFDLAVLELASPLAFNKYIQPVCLPLAIQKFPVG | 149 |
| | | \|+ \| ++++++\|\|\|\|\| \|+\|+\|+\|\|\|+ ++\|+\|\|\|\| \|\|\| | |
| Sbjct: | 59 | GTTEGTEQKFDVKKIIVHPNYNPD--TNDIALLKLKSPVTLGDTVRPICLPSASSDLPVG | 116 |
| NOV22: | 150 | RKCMISGWGNTQEGN----LQKASVGIIDQKTCSVLYNFSLTDRMICAGFLEGKVDSCQG | 205 |
| | | \|+\|\|\|\|\|+ \|\|+ \|\|+++\|\| \| ++\|\|\|\|\|\|\|\|\|\|\|+\|\|\| | |
| Sbjct: | 117 | TTCSVSGWGRTKNLGTSDTLQEVVVPIVSRETCRSAYGGTVTDTMICAGALGGK-DACQG | 175 |
| NOV22: | 206 | DSGGPLACEEAPGVFYLAGIVSWGIGCAQVKKPGVYTRITRLKGWI | 251 |
| | | \|\|\|\|\|\|\|+ \|\|\|\|\|\|\|\|\|\| \|\|\|\|\|\|++\| \|\| | |
| Sbjct: | 176 | DSGGPLVCSDG----ELVGIVSWGYGCAVGNYPGVYTRVSRYLDWI | 217 |

Proteolytic enzymes that exploit serine in their catalytic activity are ubiquitous, being found in viruses, bacteria and eukaryotes [1]. They include a wide range of peptidase activity, including exopeptidase, endopeptidase, oligopeptidase and omega-peptidase activity. Over 20 families (denoted S1-S27) of serine protease have been identified, these being grouped into 6 clans on the basis of structural similarity and other functional evidence [1].

Tryptase is a tetrameric serine protease that is concentrated and stored selectively in the secretory granules of all types of mast cells, from which it is secreted during mast cell degranulation. Its exclusive presence in mast cells permits its use as a specific clinical indicator of mast cell activation by measurement of its level in biologic fluids and as a selective marker of intact mast cells using immunohistochemical techniques with antitryptase antibodies. Vanderslice [2] demonstrated the existence of multiple tryptases. In this respect, mast cell tryptase is like other serine proteases such as glandular kallikrein and trypsin, which are also members of multigene families. Miller et al. [3] mapped both alpha-tryptase and beta-tryptase to human chromosome 16 by PCR analysis of DNA from human/hamster somatic cell hybrids. Miller et al. [3] cloned a second cDNA for human tryptase, called beta-tryptase, from a mast cell cDNA library. The 1,142 bases of beta-tryptase were found to encode a 30-amino acid leader sequence of 3,089 daltons and a 245-amino acid catalytic region of 27,458 daltons. The amino acid sequence of beta-tryptase was found to be 90% identical with that of alpha-tryptase, the first 20 amino acids of the catalytic portions being 100% identical. Both alpha- and beta-tryptase sequences were localized to human chromosome 16 by analysis of DNA preparations from 25 human/hamster somatic cell hybrids by PCR.

Because of the presence of the trypsin domains and the homology to the adrenal secretory serine protease, we anticipate that the novel sequence described here will have useful properties and functions similar to these genes.

The disclosed NOV22 nucleic acid of the invention encoding a Adrenal secretory serine protease-like protein includes the nucleic acid whose sequence is provided in Table 22A, 25C, 25E or a fragment thereof. The invention also includes a mutant or variant nucleic acid any of whose bases may be changed from the corresponding base shown in Table 22A, 25C, or 25E while still encoding a protein that maintains its Adrenal secretory serine protease-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 36 percent of the bases may be so changed.

The disclosed NOV22 protein of the invention includes the Adrenal secretory serine protease-like protein whose sequence is provided in Table 22B, 25D, or 25F. The invention also includes a mutant or variant protein any of whose residues may be changed from the corresponding residue shown in Table 22B, 25D, or 25F while still encoding a protein that maintains its Adrenal secretory serine protease-like activities and physiological functions, or a functional fragment thereof. In the mutant or variant protein, up to about 57 percent of the residues may be so changed.

The invention further encompasses antibodies and antibody fragments, such as F_{ab} or (F_{ab})₂, that bind immunospecifically to any of the proteins of the invention.

The above disclosed information suggests that this Adrenal secretory serine protease-like protein (NOV22) is a member of a “Adrenal secretory serine protease family”. Therefore, the NOV22 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV22 nucleic acids and proteins of the invention are useful in potential therapeutic applications implicated in Von Hippel-Lindau (VHL) syndrome, cirrhosis, transplantation, endometriosis, fertility, anemia, ataxia-telangiectasia, autoimmune disease, hemophilia, hypercoagulation, idiopathic thrombocytopenic purpura, allergies, immunodeficiencies, graft versus host disease (GVHD), lymphaedema, muscular dystrophy, Lesch-Nyhan syndrome, myasthenia gravis, and/or other diseases and pathologies.

NOV22 nucleic acids and polypeptides are further useful in the generation of antibodies that bind immuno-specifically to the novel NOV22 substances for use in therapeutic or diagnostic methods. These antibodies may be generated according to methods known in the art, using prediction from hydrophobicity charts, as described in the “Anti-NOVX Antibodies” section below. The disclosed NOV22 protein has multiple hydrophilic regions, each of which can be used as an immunogen. These novel proteins can be used in assay systems for functional analysis of various human disorders, which will help in understanding of pathology of the disease and development of new drug targets for various disorders.

NOV23

NOV23 includes three novel serine protease DESC1 protease-like proteins disclosed below. The disclosed sequences have been named NOV23a, NOV23b, NOV23c, and NOV23d.

NOV23a

The disclosed NOV23a nucleic acid of 1546 nucleotides (also referred to as CG56647-02) encoding a novel serine protease DESC1-like protein is shown in Table 23A. An open reading frame was identified beginning with an ATG initiation codon at nucleotides 101-103 and ending with a TAG codon at nucleotides 1481-1483. Putative untranslated regions, if any, found upstream from the initiation codon and downstream from the termination codon are underlined in Table 23A, and the start and stop codons are in bold letters.

TABLE 23A
NOV23a Nucleotide Sequence

(SEQ ID NO:97)

GCCCCTGCCATAGGAGGCGGGGACTGTCATTTCACCGTCTCCTGATGCCATTCCAGAGGTTACGCCCTGA
AGTCAGCTCAGATCCTGGGCCAGGCACTGCATGGGAGACAGGCATCAGCAGGACCTCTTTCTGCCTTCGA
GGAAAACACGGGGGCATCTGGGGCTCACTTGGCACTCATCCACCTTGTGCTGTACCTGGGGACCTCCGGC
CTTCCTCTCTACACAGGGCTTCCACGTGGACCACACGGCCGAGCTGCGGGGAATCCGGTGGACCAGCAGT
TTGCGGCGGGAGACCTCGGACTATCACCGCACGCTGACGCCCACCCTGGAGGCACTGTTTGTAAGTAGTT
TTCAGAAGACAGAGTTAGAGGCAAGCTGCGTGGGTTGCTCGGTACTGAATTATAGGGATGGGAACTCCAG
TGTCCTCGTACATTTCCAGCTGCACTTTCTGCTGCGACCCCTCCAGACGCTGAGCCTGGGCCTGGAGGAG
GAGCTATTGCAGCGAGGGATCCGGGCAAGGCTGCGGGAGCACGGCATCTCCCTGGCTGCCTATGGCACAA
TTGTGTCGGCTGAGCTCACAGGTAGACATAAGGGACCCTTGGCAGAAAGAGACTTCAAATCAGGTCGCTG
TCCAGGGAACTCCTTTTCCTGCGGGAACAGCCAGTGTGTGACCAAGGTGAACCCGGAGTGTGACGACCAG
GAGGACTGCTCCGATGGGTCCGACGAGCCGCACTGCGAGTGTGGCTTGCAGCCTGCCTGGAGGATGGCCG
GCAGGATCGTGGGCGGCATCGAAGCATCCCCGGGGGAGTTTCCGTGGCAAGCCAGCCTTCGAGAGAACAA
GGAGCACTTCTGTGGGGCCGCCATCATCAACGCCAGGTGGCTGGTGTCTGCTGCTCACTGCTTCAATGAC
TTCCAAGACCCGACGAAGTGGGTGGCCTACGTGGCTGCGACCTACCTCAGCGGCTCGGACGCCAGCACCG
TGCGCGCCCAGGTGGTCCAGATCGTCAAGCACCCCCTGTACAACGCGGACACGGCCGACTTTGACGTGGC
TGTGCTGGAGCTGACCAGCCCTCTGCCTTTCGGCCGGCACATCCAGCCCGTGTGCCTCCCGGCTGCCACA
CACATCTTCCCACCCAGCAAGAAGTGCCTGATCTCAGGCTGGGGCTACCTCAAGCACGACTTCGTGGTCA
AGCCAGAGGTGCTGCAGAAAGCCACTGTGCAGCTGCTGGACCAGGCACTGTGTGCCAGCTTGTACGGCCA
TTCACTCACTGACAGGATCGTGTGCCCTGGCTACCTGGACGGGAAGGTGGACTCCTGCCAGCGTGACTCA
GGAGGACCCCTGGTCTGCGAGGAGCCCTCTGGCCGGTTCTTTCTGGCTGGCATCGTGAGCTGGGGAATCG
GGTGTGCGGAAGCCCGGCGTCCAGGGGTCTATGCCCGAGTCACCAGGCTACGTGACTGGATCCTGGAGGC
CACCGAAAGGTAGAAGATCATGTACCTGCCTATCTTGATTTAGGGAGAACGGATATCGTCATAGTATCTT
CATAAT

The disclosed NOV23a nucleic acid sequence, located on chromosome 19, has 356 of 566 bases (62%) identical to a gb:GENBANK-ID:AF133086|acc:AF133086.1 mRNA from Homo sapiens (membrane-type serine protease 1 mRNA, complete cds) (E=1.1e⁻²³).

A disclosed NOV23a polypeptide (SEQ ID NO:98) encoded by SEQ ID NO:97 is 460 amino acid residues and is presented using the one-letter amino acid code in Table 23B. Signal P, Psort and/or Hydropathy results predict that NOV23a contains no signal peptide and is likely to be localized in the microbody (peroxisome) with a certainty of 0.5387.

TABLE 23B
Encoded NOV23a protein sequence.

(SEQ ID NO:98)

MGDRHEQDLFLPSRKTRGHLGLTWHSSTLCCTWGPPAFLSTQGFHVDHTAELRGIRWTSSLRRETSDYHRTLTPT
LEALFVSSFQKTELEASCVGCSVLNYRDGNSSVLVHFQLHFLLRPLQTLSLGLEEELLQRGIRARLREHGISLAA
YGTIVSAELTGRHKGPLAERDFKSGRCPGNSFSCGNSQCVTKVNPECDDQEDCSDGSDEAHCECGLQPAWRMAGR
IVGGMEASPGEFPWQASLRENKEHFCGAAIINARWLVSAAHCFNEFQDPTKWVAYVGATYLSGSEASTVRAQVVQ
IVKHPLYNADTADFDVAVLELTSPLPFGRHIQPVCLPAATHTFPPSKKCLISGWGYLKEDFVVKPEVLQKATVEL
LDQALCASLYGHSLTDRMVCAGYLDGKVDSCQGDSGGPLVCEEPSGRFFLAGIVSWGIGCAEARRPGVYARVTRL
RDWILEATER

The disclosed NOV23a amino acid sequence has 112 of 248 amino acid residues (45%) identical to, and 157 of 248 amino acid residues (63%) similar to, the 422 amino acid residue ptnr:SPTREMBL-ACC:Q9UL52 protein from Homo sapiens (Human) (serine protease DESC1) (E=1.1e⁻⁵⁸).

NOV23a is predicted to be expressed in at least Ovary, kidney, breast, lung, muscle, liver, spleen, blood and lymphocyte. This information was derived by determining the tissue sources of the sequences that were included in the invention including but not limited to SeqCalling sources, Public EST sources, and/or RACE sources.

NOV23b

A disclosed NOV23b nucleic acid of 1777 nucleotides (also referred to as CG56647-03) encoding a novel serine protease DESC1-like protein is shown in Table 23C. An open reading frame was identified beginning with an ATG initiation codon at nucleotides 101-103 and ending with a TAG codon at nucleotides 1631-1633. Putative untranslated regions, if any, found upstream from the initiation codon and downstream from the termination codon are underlined in Table 23C, and the start and stop codons are in bold letters.

TABLE 23C
NOV23b Nucleotide Sequence

(SEQ ID NO:99)

GCCCCTGCCATAGGAGGCGGGGACTGTCATTTCACCGTCTCCTGATGCCATTCCAGAGGTTACGCCCTGA
AGTCAGCTCAGATCCTGGGCCACGCACTGCATGGGAGACAGGCATCAGCAGGACCTCTTTCTGCCTTCGA
GGAAAACACGGGGGCATCTGGGGCTCACTTGGCACTCATCCACCTTGTGCTGTACCTGGCGACCTCCGGC
CTTCCTCTCTACACAGGGCTTCCACGTGGACCACACGGCCGAGCTGCGGGGAATCCGGTGGACCAGCAGT
TTGCGGCGGGAGACCTCGGACTATCACCGCACGCTGACGCCCACCCTGGAGGCACTGTTTGTAAGTAGTT
TTCAGAAGACAGAGTTAGAGGCAAGCTGCGTGGGTTGCTCCGTACTGAATTATAGGGATGGGAACTCCAG
TGTCCTCGTACATTTCCAGCTGCACTTTCTGCTGCGACCCCTCCAGACGCTGAGCCTGGGCCTGGAGGAG
GACCTATTCCAGCGAGGGATCCGGGCAAGGCTGCGGGAGCACGGCATCTCCCTGGCTGCCTATGGCACAA
TTGTGTCGGCTGAGCTCACAGGGAGACATAAGCGACCCTTGGCAGAAAGAGACTTCAAATCACGCCGCTG
TCCACGCAACTCCTTTTCCTGCGGGAACAGCCAGTGTGTCACCAAGGTGAACCCGGAGTGTGACGACCAG
GAGGACTGCTCCGATGGGTCCGACGAGGCGCACTGCGAGTGTGGCTTGCAGCCTGCCTGGAGGATGGCCG
GCAGGATCGTGGGCGGCATGGAAGCATCCCCGGGGCAGTTTCCGTGGCAAGCCAGCCTTCGAGAGAACAA
GGAGCACTTCTGTGGGGCCGCCATCATCAACGCCAGGTGGCTGGTGTCTCCTGCTCACTGCTTCAATGAG
TTCCAAGACCCGACGAAGTGGGTGGCCTACGTCGGTGCGACCTACCTCAGCGGCTCGGAGCCCAGCACCG
TGCGGGCCCAGGTGGTCCAGATCGTCAAGCACCCCCTGTACAACGCGGACACGGCCGACTTTGACGTGGC
TCTGCTGGAGCTGACCAGCCCTCTGCCTTTCGGCCGGCACATCCAGCCCGTGTGCCTCCCGGCTGCCACA
CACATCTTCCCACCCAGCAAGAAGTGCCTGATCTCAGGCTGGGGCTACGTGCTGCAGAAAGCCACTGTGG
AGCTGCTGGACCAGGCACTGTGTGCCAGCTTGTACGGCCATTCACTCACTGACAGGATGGTGTGCGCTGG
CTACCTGGACGGGAAGGTGGACTCCTGCCAGGGTGACTCAGGAGGACCCCTGGTCTGCGAGGAGCCCTCT
GGCCGGTTGTTTCTGGCTGGCATCGTGAGCTGGGGAATCCGGTGTGCGGAAGCCCGGCATCCAGGGGTCT
ATGCCCGAGTCACCAGGCTACGCGACTGGATCCTGGAGGCCACCACCAAAGCCAGCATGCCTCTGGCCCC
CACCATGGCTCCTGCCCCTGCCGCCCCCAGCACAGCCTGGCCCACCAGTCCTGACAGCCCTGTGGTCAGC
ACCCCCACCAAATCGATGCAGGCCCTCAGTACCGTGCCTCTTGACTGGGTCACCGTTCCTAAGCTACAAG
GTATTTTCGGGGCAGAAAGGTAGAAGATGATGTACGTGCCTATCTTCATTTAGGGAGAACGGATATCGTC
ATAGTATCTTCATAATTTTGGATCTTCCTGTTCAAGGAAAGGTCACATGTGTATCCGTTTATTCCCATCT
TACGTTGCGTGTACCCTCATGGTATCT

The disclosed NOV23b nucleic acid sequence, located on chromosome 19, has 208 of 327 bases (63%) identical to a gb:GENBANK-ID:AF098327|acc:AF098327.1 mRNA from Homo sapiens (putative mast cell mMCP-7-like II typtase gene, complete cds) (E=2.8e⁻¹⁴)

A disclosed NOV23b polypeptide (SEQ ID NO:100) encoded by SEQ ID NO:99 is 510 amino acid residues and is presented using the one-letter amino acid code in Table 23D. Signal P, Psort and/or Hydropathy results predict that NOV23b contains no signal peptide and is likely to be localized in the microbody (peroxisome) with a certainty of 0.5131.

TABLE 23D
Encoded NOV23b protein sequence.

(SEQ ID NO:100)

MGDRHEQDLFLPSRKTRGHLGLTWHSSTLCCTWGPPAFLSTQGFHVDHTAELRGIRWTSSLRRETSDYHRTLTPT
LEALFVSSFQKTELEASCVGCSVLNYRDGNSSVLVHFQLHFLLRPLQTLSLGLEEELLQRGIRARLREHGISLAA
YGTIVSAELTGRHKGPLAERDFKSGRCPGNSFSCGNSQCVTKVNPECDDQEDCSDGSDEAHCECGLQPAWRMAGR
IVGGMEASPGEFPWQASLRENKEHFCGAAIINARWLVSAAHCFNEFQDPTKWVAYVGATYLSGSEASTVPAQVVQ
IVKHPLYNADTADFDVAVLELTSPLPFGRHIQPVCLPAATHIFPPSKKCLISGWGYVLQKATVELLDQALCASLY
GHSLTDRMVCAGYLDGKVDSCQGDSGGPLVCEEPSGRLFLAGIVSWGIGCAEARHPGVYARVTRLRDWILEATTK
ASMPLAPTMAPAPAAPSTAWPTSPESPVVSTPTKSMQALSTVPLDWVTVPKLQGIFGAER

The disclosed NOV23b amino acid sequence has 109 of 246 amino acid residues (44%) identical to, and 152 of 246 amino acid residues (61%) similar to, the 422 amino acid residue ptnr:SPTREMBL-ACC:Q9UL52 protein from Homo sapiens (Human) (serine protease DESC1) (E=1.3e⁻⁵⁵).

NOV23b is predicted to be expressed in at least the following tissues: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea and uterus. Expression information was derived from the tissue sources of the sequences that were included in the derivation of the NOV23b sequence.

NOV23c

A disclosed NOV23c nucleic acid of 815 nucleotides (also referred to as CG56647-01) encoding a novel adrenal secretory serine protease-like protein is shown in Table 23E. An open reading frame was identified beginning with a GGT initiation codon at nucleotides 1-3 and ending with a TAA codon at nucleotides 787-789. The start and stop codons are shown in bold in Table 23E, and the 5′ and 3′ untranslated regions, if any, are underlined. Because the start codon of NOV23c is not a traditional initiation codon, NOV23c could be a partial reading frame that extends further in the 5′ direction.

TABLE 23E
NOV23c nucleotide sequence.

(SEQ ID NO:101)

GGTCCTGCCTTCGTGGGGTCATGGCTCGTGACCTGCTGTCTTGCAGAGTGTGGCTTGCAGCCTGCCTGGAGG
ATGGCCGGCAGGATCGTGGGCGGCATGGAAGCATCCCCGGGGGAGTTTCCGTGGCAAGCCAGCCTTCGAGAG
AACAAGGAGCACTTCTGTGGGGCCGCCATCATCAACGCCAGGTGGCTGGTGTCTGCTGCTCACTGCTTCAAT
GAGTTCCAACACCCCACGAAGTGGGTGGCCTACGTGGGTGCGACCTACCTCAGCGGCTCGGAGGCCAGCACC
GTCCGGGCCCAGGTGGTCCAGATCGTCAAGCACCCCCTGTACAACGCGGACACGGCCGACTTTGACGTGGCT
GTGCTGGAGCTGACCAGCCCTCTGCCTTTCGGCCGGCACATCCAGCCCGTGTGCCTCCCGGCTGCCACACAC
ATCTTCCCACCCAGCAAGAAGTGCCTGATCTCAGGCTGGGGCTACCTCAAGGAGGACTTCCGTAAGCATCTT
CCTCTGCAGAAAGCCACTGTGGAGCTGCTGGACCAGGCACTGTGTGCCAGCTTGTACGGCCATTCACTCACT
GACAGGATGGTGTGCGCTGGCTACCTGGACGGGAAQGTGGACTCCTGCCAGGGTGACTCAGGAGGACCCCTG
GTCTGCGAGGAGCCCTCTGGCCGGTTCTTTCTGGCTGGCATCCTGAGCTGGGGAATCGGGTGTGCGGAAGCC
CGGCGTCCAGGGGTCTATGCCCGAGTCACCAGGCTACGTGACTGGATCCTGGAGGCCACCCGTTCCTAAGCT
ACAACGTATTTTCGGGGCAGAAA

In a search of public sequence databases, the NOV23c nucleic acid sequence, located on chromosome 19, has 350 of 564 bases (62%) identical to a gb:GENBANK-ID:E13204|acc:E13204.1 mRNA from Homo sapiens (Human cDNA encoding a serine protease) (E=3.2e⁻²⁶).

The disclosed NOV23c polypeptide (SEQ ID NO:102) encoded by SEQ ID NO:101 has 262 amino acid residues and is presented in Table 23F using the one-letter amino acid code. Signal P, Psort and/or Hydropathy results predict that NOV23c has no signal peptide and is likely to be localized extracellularly with a certainty of 0.3750. Alternatively, NOV23c may also localize to the microbody (peroxisome) with a certainty of 0.1391, to the endoplasmic reticulum (membrane) with a certainty of 0.1000, or the endoplasmic reticulum (lumen) with a certainty of 0.1000. The most likely cleavage site for NOV23c is between positions 15 and 16: CLA-EC.

TABLE 23F
Encoded NOV23c protein sequence.

(SEQ ID NO:102)

GPAFVGSWLVTCCLAECGLQPAWRMAGRIVGGMEASPGEFPWQASLRENKEHFCGAAIINARWLVSAAHCFN
EFQDPTKWVAYVGATYLSGSEASTVRAQVVQIVKHPLYNADTADFDVAVLELTSPLPFGRHIQPVCLPAATH
IFPPSKKCLISGWGYLKEDFRKHLPLQKATVELLDQALCASLYGHSLTDRMVCAGYLDGKVDSCQGDSGGPL
VCEEPSGRFFLAGIVSWGIGCAEARRPGVYARVTRLRDWILEATRS

A search of sequence databases reveals that the NOV23c amino acid sequence has 114 of 248 amino acid residues (45%) identical to, and 152 of 248 amino acid residues (61%) similar to, the 273 amino acid residue ptnr:TREMBLNEW-ACC:BAB20278 protein from Mus musculus (Mouse) (Type 1 Spinesin) (E=1.1e⁻⁵³).

NOV23c is predicted to be expressed in at least the following tissues: Ovary, kidney, breast, lung, muscle, liver, spleen, blood, lymphocyte. This information was derived by determining the tissue sources of the sequences that were included in the invention including but not limited to SeqCalling sources, Public EST sources, Literature sources, and/or RACE sources.

The disclosed NOV23a polypeptide has homology to the amino acid sequences shown in the BLASTP data listed in Table 23G.

TABLE 23G
BLAST results for NOV23

| Gene Index/ | | Length | Identity | Positives | |
|---|---|---|---|---|---|
| Identifier | Protein/Organism | (aa) | (%) | (%) | Expect |
| gi\|12836503\|dbj\|BAB23684.1\| | data source: SPTR, | 799 | 136/280 | 180/280 | 1e−75 |
| (AK004939) | source | | (48%) | (63%) | |
| | key: O95519, | | | | |
| | evidence: ISS˜homo | | | | |
| | log to DJ1170K4.4 | | | | |
| | (NOVEL PROTEIN) | | | | |
| | (FRAGMENT)˜putative | | | | |
| | [Mus musculus] | | | | |
| gi\|16758444\|ref\|NP_{—} | suppression of | 855 | 133/289 | 182/289 | 3e−72 |
| 446087.1\| | tumorigenicity 14 | | (46%) | (62%) | |
| (NM_053635) | (colon carcinoma, | | | | |
| | matriptase, | | | | |
| | epithin) [Rattus | | | | |
| | norvegicus] | | | | |
| gi\|10257390\|gb\|AAG15395.1\| | serine protease | 855 | 132/289 | 185/289 | 3e−72 |
| AF057145_1 | TADG15 [Homo | | (45%) | (63%) | |
| (AF057145) | sapiens] | | | | |
| gi\|11415040\|ref\|NP_{—} | suppression of | 855 | 132/289 | 185/289 | 3e−72 |
| 068813.1\| | tumorigenicity 14 | | (45%) | (63%) | |
| (NM_021978) | (colon carcinoma, | | | | |
| | matriptase, | | | | |
| | epithin); | | | | |
| | suppression of | | | | |
| | tumorigenicity 14 | | | | |
| | (colon | | | | |
| | carcinoma); | | | | |
| | matriptase [Homo | | | | |
| | sapiens] | | | | |
| gi\|12249015\|dbj\|BAB20376.1\| | prostamin [Homo | 855 | 131/289 | 184/289 | 9e−72 |
| (AB030036) | sapiens] | | (45%) | (63%) | |

The homology between these and other sequences is shown graphically in the ClustalW analysis shown in Table 23H. In the ClustalW alignment of the NOV23 protein, as well as all other ClustalW analyses herein, the black outlined amino acid residues indicate regions of conserved sequence (i.e., regions that may be required to preserve structural or functional properties), whereas non-highlighted amino acid residues are less conserved and can potentially be altered to a much broader extent without altering protein structure or function.

[image]

[image]

[image]

Tables 23I-L list the domain descriptions from DOMAIN analysis results against NOV23. This indicates that the NOV23 sequence has properties similar to those of other proteins known to contain this domain.

TABLE 23I
Domain Analysis of NOV23a

gnl|Smart|smart00020, Tryp_SPc, Trypsin-like serine protease; Many of
these are synthesised as inactive precursor zymogens that are cleaved
during limited proteolysis to generate their active forms. A few,
however, are active as single chain molecules, and others are inactive
due to suhstitutions of the catalytic triad residues. (SEQ ID NO:812)
CD-Length = 230 residues, 100.0% aligned
Score = 269 bits (687), Expect = 3e−73

| | | | |
|---|---|---|---|
| NOV23: | 225 | RIVGGMEASPGEFPWQASLR-ENKEHFCGAAIINARWLVSAAHCFNEFQDPTKWVAYVGA | 283 |
| | | \|\|\|\|\|\|\|+\|\|\|\|\|\|\|+ \|\|\|\|++\|+\|\|+++\|\|\|\| \|+ +\|+ | |
| Sbjct: | 1 | RIVGGSEANIGSFPWQVSLQYRGGRHFCGGSLISPRWVLTAAHCVYGSA-PSSIRVRLGS | 59 |
| NOV23: | 284 | TYLSGSEASTVRAQVVQIVKHPLYNADTADFDVAVLELTSPLPFGRHIQPVCLPAATHIF | 343 |
| | | \|\| \|+ +\|+++\|\|\|\| \|\|\|\|+\|+\|+ ++\|+\|\|\|+++ | |
| Sbjct: | 60 | HDLSSGEETQTV-KVSKVIVHPNYNPSTYDNDIALLKLSEPVTLSDTVRPICLPSSGYNV | 118 |
| NOV23: | 344 | PPSKKCLISGWGYLKEDFVVKPEVLQKATVELLDQALCASLY--GHSLTDRMVCAGYLDG | 401 |
| | | \| \|+\|\|\|\| \| \|+\|\|+ \|++ \|\| \| \|++\|\|\|+\|\|\|\|+\| | |
| Sbjct: | 119 | PAGTTCTVSGWGRTSESSGSLPDTLQEVNVPIVSNATCRRAYSGGPAITDNMLCAGGLEG | 178 |
| NOV23: | 402 | KVDSCQGDSGGPLVCEEPSGRFFLAGIVSWG-IGCAEARRPGVYARVTRLRDWI | 454 |
| | | \|+\|\|\|\|\|\|\|\|\|\|\| \|+\|\|\|\|\|\|\| \|\|\| +\|\|\|\|\|\|+ \|\|\| | |
| Sbjct: | 179 | GKDACQGDSGGPLVCN--DPRWVLVGIVSWGSYGCARPNKPGVYTRVSSYLDWI | 230 |

TABLE 23J
Domain Analysis of NOV23a

gnl|Pfam|pfam00089, trypsin, Trypsin. Proteins recognized include all
proteins in families S1, S2A, S2B, S2C, and S5 in the classification
of peptidases. Also included are proteins that are clearly members,
but that lack peptidase activity, such as haptoglobin and protein Z
(PRTZ*). (SEQ ID NO:813)
CD-Length = 217 residues, 100.0% aligned
Score = 223 bits (568), Expect = 2e−59

| | | | |
|---|---|---|---|
| NOV27: | 226 | IVGGMEASPGEFPWQASLRENKEHFCGAAIINARWLVSAAHCFNEFQDPTKWVAYVGATY | 285 |
| | | \|\|\|\|\|\| \|\|\|\|\|\|\|++ \|\|\|\|++\|+ \|+++\|\|\|\|+ +\| +\| | |
| Sbjct: | 1 | IVGGREAQAGSFPWQVSLQVSSGHFCGGSLISENWVLTAAHCVS---GASSVRVVLGEHN | 57 |
| NOV27: | 286 | LSGSEASTVRAQVVQIVKHPLYNADTADFDVAVLELTSPLPFGRHIQPVCLPAATHIFPP | 345 |
| | | \| +\|+ + \|+\|+\|\|\|\|\|\| \|+\|+\|+\|\|\|+ \| ++\|+\|\|\|+\|+ \| | |
| Sbjct: | 58 | LGTTEGTEQKFDVKKIIVHPNYNPDT--NDIALLKLRSPVTLGDTVRPICLPSASSDLPV | 115 |
| NOV27: | 346 | SKKCLISGWGYLKEDFVVKPEVLQKATVELLDQALCASLYGHSLTDRMVCAGYLDGKVDS | 405 |
| | | \|+\|\|\|\| \| +\|\|+ \|+++ \|\|\|\|++\|\|\|+\|\|\|\|\|\|\|+ | |
| Sbjct: | 116 | GTTCSVSGWGRTKNL--GTSDTLQEVVVPIVSRETCRSAYGGTVTDTMICAGALGGK-DA | 172 |
| NOV27: | 406 | CQGDSGGPLVCEEPSGRFFLAGIVSWGIGCAEARRPGVYARVTRLRDWI | 454 |
| | | \|\|\|\|\|\|\|\|\|\|\|+ \|\|\|\|\|\|\|\|\|\| \|\|\|\|\|\|+\| \|\|\| | |
| Sbjct: | 173 | CQGDSGGPLVCSDG----ELVGIVSWGYGCAVGNYPGVYTRVSRYLDWI | 217 |

TABLE 23K
Domain Analysis of NOV23b

gnl|Smart|smart00192, LDLa, Low-density lipoprotein receptor
domain class A; Cysteine-rich repeat in the low-density
lipoprotein (LDL) receptor that plays a central role in
mammalian cholesterol metabolism. The N-terminal type A
repeats in LDL receptor bind the lipoproteins. Other
homologous domains occur in related receptors, including the
very low-density lipoprotein receptor and the LDL receptor-
related protein/alpha 2-macroglobulin receptor, and in
proteins which are functionally unrelated, such as the C9
component of complement. Mutations in the LDL receptor gene
cause familial hypercholesterolemia. (SEQ ID NO:814)
CD-Length = 38 residues, 100.0% aligned
Score = 50.4 bits (119), Expect = 2e−07

| | | | |
|---|---|---|---|
| NOV23: | 176 | RCPGNSFSCGNSQCVTKVNPECDDQEDCSDGSDEAHCEC | 214 |
| | | \|\| \|\|\|+\|+ \|\| +\|\|\|\|\|\|\|+\| | |
| Sbjct: | 1 | TCPPGEFQCKNGRCIPLSWV-CDGVDDCGDGSDEENCPS | 38 |

TABLE 23L
Domain Analysis of NOV23b

gnl|Pfam|pfam00057, ldl_recept_a, Low-density lipoprotein
receptor domain class A (SEQ ID NO:8lS)
CD-Length = 39 residues, 94.9% aligned
Score = 43.5 bits (101), Expect = 3e−05

| | | | |
|---|---|---|---|
| NOV23: | 175 | GRCPGNSFSCGNSQCVTKVNPECDDQEDCSDGSDEAHC | 212 |
| | | \| \|\|\|\|++\|+ \|\| \|\|\|\|\|\|\|+\| | |
| Sbjct: | 1 | STCGPNEFQCGSGECIPMSW-VCDGDPDCEDGSDEKNC | 37 |

Proteolytic enzymes that exploit serine in their catalytic activity are ubiquitous, being found in viruses, bacteria and eukaryotes. They include a wide range of peptidase activity, including exopeptidase, endopeptidase, oligopeptidase and omega-peptidase activity. Over 20 families (denoted S1-S27) of serine protease have been identified, these being grouped into 6 clans on the basis of structural similarity and other functional evidence.

Tryptase is a tetrameric serine protease that is concentrated and stored selectively in the secretory granules of all types of mast cells, from which it is secreted during mast cell degranulation. Its exclusive presence in mast cells permits its use as a specific clinical indicator of mast cell activation by measurement of its level in biologic fluids and as a selective marker of intact mast cells using immunohistochemical techniques with antitryptase antibodies. Vanderslice demonstrated the existence of multiple tryptases. In this respect, mast cell tryptase is like other serine proteases such as glandular kallikrein and trypsin, which are also members of multigene families. Miller et al. mapped both alpha-tryptase and beta-tryptase to human chromosome 16 by PCR analysis of DNA from human/hamster somatic cell hybrids. Miller et al. cloned a second cDNA for human tryptase, called beta-tryptase, from a mast cell cDNA library. The 1,142 bases of beta-tryptase were found to encode a 30-amino acid leader sequence of 3,089 daltons and a 245-amino acid catalytic region of 27,458 daltons. The amino acid sequence of beta-tryptase was found to be 90% identical with that of alpha-tryptase, the first 20 amino acids of the catalytic portions being 100% identical. Both alpha- and beta-tryptase sequences were localized to human chromosome 16 by analysis of DNA preparations from 25 human/hamster somatic cell hybrids by PCR.

Because of the presence of the trypsin domains and the homology to the adrenal secretory serine protease, it is anticipated that the novel sequences described here will have useful properties and functions similar to these proteins.

The disclosed NOV23 nucleic acid of the invention encoding an Adrenal secretory serine protease-like protein includes the nucleic acids whose sequences are provided in Tables 23A, 23C, 23E or a fragment thereof. The invention also includes a mutant or variant nucleic acid any of whose bases may be changed from the corresponding base shown in Table 23A, 23C, or 23E while still encoding a protein that maintains its Adrenal secretory serine protease-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 37 percent of the bases may be so changed.

The disclosed NOV23 protein of the invention includes the Adrenal secretory serine protease-like protein whose sequence are provided in Table 23B, 23D, or 23F. The invention also includes a mutant or variant protein any of whose residues may be changed from the corresponding residue shown in Table 23B, 23D, or 23F while still encoding a protein that maintains its Adrenal secretory serine protease-like activities and physiological functions, or a functional fragment thereof. In the mutant or variant protein, up to about 55 percent of the residues may be so changed.

The invention further encompasses antibodies and antibody fragments, such as F_{ab} or (F_{ab})₂, that bind immunospecifically to any of the proteins of the invention.

The above disclosed information suggests that these Adrenal secretory serine protease-like proteins (NOV23) is a member of a “Adrenal secretory serine protease family”. Therefore, the NOV23 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV23 nucleic acids and proteins of the invention are useful in potential therapeutic applications implicated in Von Hippel-Lindau (VHL) syndrome, cirrhosis, transplantation, endometriosis, fertility, anemia, ataxia-telangiectasia, autoimmune disease, hemophilia, hypercoagulation, idiopathic thrombocytopenic purpura, allergies, immunodeficiencies, graft versus host disease (GVHD), lymphaedema, muscular dystrophy, Lesch-Nyhan syndrome, myasthenia gravis, and/or other diseases and pathologies.

NOV23 nucleic acids and polypeptides are further useful in the generation of antibodies that bind immuno-specifically to the novel NOV23 substances for use in therapeutic or diagnostic methods. These antibodies may be generated according to methods known in the art, using prediction from hydrophobicity charts, as described in the “Anti-NOVX Antibodies” section below. The disclosed NOV23 protein has multiple hydrophilic regions, each of which can be used as an immunogen. These novel proteins can be used in assay systems for functional analysis of various human disorders, which will help in understanding of pathology of the disease and development of new drug targets for various disorders.

NOV24

NOV24 includes two novel parchorin-like proteins disclosed below. The disclosed sequences have been named NOV24a and NOV24b.

NOV24a

A disclosed NOV24a nucleic acid of 2091 nucleotides (also referred to as CG56455-01) encoding a novel parchorin-like protein is shown in Table 24A. An open reading frame was identified beginning with a ATG initiation codon at nucleotides 7-9 and ending with a TGA codon at nucleotides 2080-2082. The start and stop codons are shown in bold in Table 24A, and the 5′ and 3′ untranslated regions, if any, are underlined.

TABLE 24A

NOV24a nucleotide sequence.

(SEQ ID NO:103)

GCGGCCATGGCCGAGGCCGCGGAGCCGGAGGGGGTTGCCCCGGGTCCCCAGGGGCCGCCGGAGGTCCCCGCG
CCTCTGGCTGAGAGACCCGGAGAGCCAGGAGCCGCGGGCGCGGAGGCAGAAGGGCCGGAGGGGAGCGAGGGC
GCAGAGGAGGCGCCGAGGGGCGCCGCCGCTGTGAAGGAGGCAGGAGGCGGCGGGCCAGACAGGGGCCCGGAG
GCCGAGGCGCGGGGCACGAGGGGGGCGCACGGCGAGACTGAGGCCGAGGAGGGAGCCCCGGAGGGTGCCGAG
GTGCCCCAAGGAGGGGAGGAGACAAGCGGCGCGCAGCAGGTGGAGGGGGCGAGCCCGGGACGCGGCGCGCAG
GGCGAGCCCCGCGGGGAGGCTCAGAGGGAGCCCGAGGACTCTGCGGCCCCCGAGAGGCAGGAGGAGGCGGAG
CAGAGGCCTGAGGTCCCGGAAGGTAGCGCGTCCGGGGAGGCGGGGGACAGCGTAGACGCGGAGGGCCCGCTG
GGGGACAACATAGAAGCGGAGGGCCCGGCGGGCGACAGCGTAGAGGCGGAGGGCCGGGTGGGGGACAGCGTA
GACGCGGAAGGTCCGGCGGGGGACAGCGTAGACGCGGAGGGCCCGCTGGGGGACAACATACAAGCCGAGGGC
CCGGCGGGGGACAGCGTAGACGCGGAGGGCCGGGTGGGGGACAGCGTAGACGCGGAAGGTCCGGCGGGGGAC
AGCGTAGACGCGGAGGGCCGGGTGGGGGACAGCGTAGAGGCGGGGGACCCGGCGGGGGACGGCGTAGAAGCG
GGGGTCCCGGCGGGGGACAGCGTAGAAGCCGAAGGCCCGGCGGGGGACAGCATGGACGCCGAGGGTCCGGCA
GGAAGGGCGCGCCGGGTCTCGGGTGAGCCGCAGCAATCGGGGGACGGCAGCCTCTCGCCCCAGGCCGAGGCA
ATTGAGGTCGCAGCCGGGGAGAGTGCGGGGCGCAGCCCCGGTGAGCTCGCCTGGGACGCAGCGGAGGAGGCG
GAGGTCCCGGGGGTAAAGGGGTCCGAAGAAGCGGCCCCCGGGGACGCAAGGGCAGACGCTGGCGAGGACAGG
GAGGAGGAAGCAGCGGGGGGCGAAGAGGAATCCCCCGACAGCAGCCCACATGGCCAGGCCTCCAGGGGCGCC
GCGGAGCCTGAGGCCCAGCTCAGCAACCACCTGGCCGAGGAGGGCCCCGCCGAGGGTAGCGGCGAGGCCGCG
CGCGTGAACGGCCGCCGGGAGGACGGAGAGGCGTCCGAGCCCCGGGCCCTGGGGCAGGAGCACGACATCACC
CTCTTCGTCAAGGCTGGTTATGATGGTGAGAGTATCGGAAATTGCCCGTTTTCTCAGCGTCTCTTTATGATT
CTCTGGCTGAAAGGCGTTATATTTAATGTGACCACAGTGGACCTGAAAAGGAAACCCGCAGACCTGCAGAAC
CTGGCTCCCGGAACAAACCCTCCTTTCATGACTTTTGATGGTGAAGTCAAGACGGATGTGAATAAGATCGAG
GAGTTCTTAGAGGAGAAATTAGCTCCCCCGAGGTATCCCAAGCTGGGGACCCAACATCCCGAATCTAATTCC
GCAGGAAATGACGTGTTTGCCAAATTCTCAGCGTTTATAAAAAACACGAAGAAGGATGCAAATGAGGTTCAT
GAAAAGAACCTGCTGAAGGCCCTGAGGAAGCTGGATAATTACTTAAATAGCCCTCTGCCTGATGAAATAGAT
GCCTACAGCACCGAGGATGTCACTGTTTCTGGAAGGAAGTTTCTGGATGGGGACGAGCTGACGCTGGCTGAC
TGCAACCTCTTACCCAAGCTCCATATTATTAAGGTTCTTCATTTTCAGATTGTGGCCAAGAAGTACAGAGAT
TTTGAATTTCCTTCTGAAATGACTGGCATCTGGAGATACTTGAATAATGCTTATGCTAGAGATGAGTTCACA
AATACGTGTCCAGCTGATCAAGAGATTGAACACGCATATTCAGATGTTGCAAAAAGAATGAAATGAAGCTGG
GCT

In a search of public sequence databases, the NOV24a nucleic acid sequence, located on chromosome 21, has 1347 of 1897 bases (71%) identical to a gb:GENBANK-ID:AB035520|acc:AB035520.1 mRNA from Oryctolagus cuniculus (Oryctolagus cuniculus mRNA for parchorin, complete cds) (E=2.4e⁻¹⁷⁵).

A disclosed NOV24a polypeptide (SEQ ID NO:104) encoded by SEQ ID NO:103 has 691 amino acid residues and is presented in Table 24B using the one-letter amino acid code. Signal P, Psort and/or Hydropathy results predict that NOV24a has no signal peptide and is likely to be localized to the nucleus with a certainty of 0.3000. Alternatively, NOV24a may also localize to the mitochondrial matrix space with a certainty of 0.1000, or to the lysosome (lumen) with a certainty of 0.1000.

TABLE 24B

Encoded NOV24a protein sequence.

(SEQ ID NO:104)

MAEAAEPEGVAPGPQGPPEVPAPLAERPGEPGAAGGEAEGPEGSEGAEEAPRGAAAVKEAGGGGPDRGPEAE
ARGTRGAHGETEAEEGAPEGAEVPQGGEETSGAQQVEGASPGRGAQGEPRGEAQREPEDSAAPERQEEAEQR
PEVPEGSASGEAGDSVDAEGPLGDNIEAEGPAGDSVEAEGRVGDSVDAEGPAGDSVDAEGPLGDNIQAEGPA
GDSVDAEGRVGDSVDAEGPAGDSVDAEGRVGDSVEAGDPAGDGVEAGVPAGDSVEAEGPAGDSMDAEGPAGR
ARRVSGEPQQSGDGSLSPQAEAIEVAAGESAGRSPGELAWDAAEEAEVPGVKGSEEAAPGDARADAGEDRVG
DGPQQEPGEDEERRERSPEGPREEEAAGGEEESPDSSPHGEASRGAAEPEAQLSNHLAEEGPAEGSGEAARV
NGRREDGEASEPRALGQEHDITLFVKAGYDGESIGNCPFSQRLFMILWLKGVIFNVTTVDLKRKPADLQNLA
PGTNPPFMTFDGEVKTDVNKIEEFLEEKLAPPRYPKLGTQHPESNSAGNDVFAKFSAFIKNTKKDANEVHEK
NLLKALRKLDNYLNSPLPDEIDAYSTEDVTVSGRKFLDGDELTLADCNLLPKLHIIKVLHFQIVAKKYRDFE
FPSEMTGIWRYLNNAYARDEFTNTCPADQEIEHAYSDVAKRMK

A search of sequence databases reveals that the NOV24a amino acid sequence has 414 of 655 amino acid residues (63%) identical to, and 453 of 655 amino acid residues (69%) similar to, the 637 amino acid residue ptnr:SPTREMBL-ACC:Q9N2G5 protein from Oryctolagus cuniculus (Rabbit) (Parchorin) (E=2.5e⁻⁸²).

NOV24a is predicted to be expressed in at least the following tissues: brain, lung, and kidney. This information was derived by determining the tissue sources of the sequences that were included in the invention including but not limited to SeqCalling sources, Public EST sources, Literature sources, and/or RACE sources.

In addition, the sequence is predicted to be expressed in gastric parietal cells, choroid plexus, salivary duct, lacrimal gland, kidney, airway epithelia and chorioretinal epithelia because of the expression pattern of (GENBANK-ID: gb:GENBANK-ID:AB035520|acc:AB035520.1) a closely related Oryctolagus cuniculus mRNA for parchorin, complete cds homolog.

NOV24b

A disclosed NOV24b nucleic acid of 859 nucleotides (also referred to as CG56455-02) encoding a novel parchorin-like protein is shown in Table 24C. An open reading frame was identified beginning with an ATG initiation codon at nucleotides 1-3 and ending with a TGA codon at nucleotides 853-855. The start and stop codons are shown in bold in Table 24A, and the 5′ and 3′ untranslated regions, if any, are underlined.

TABLE 24C

NOV24b nucleotide sequence.

(SEQ ID NO:105)

ATGGCCGAGGCCGCGGAGCCTGAGGCCCAGCTCAGCAACCACCTGGCCGAGGAGGGCCCCGCCGAGGGTAGC
GGCGAGGCCGCGCGTGTGAACGGCCGCCGGGAGGACGGAGAGGCGTCCGAGCCCCGGGCCCTGGGGCAGGAG
CACGACATCACCCTCTTCGTCAAGGCTGGTTATGATGGTGAGAGTATCGGAAATTGCCCGTTTTCTCAGCGT
CTCTTTATGATTCTCTGGCTGAAAGGCGTTATATTTAATGTGACCACAGTGGACCTGAAAAGGAAACCCGCA
GACCTGCAGAACCTGGCTCCCGGAACAAACCCTCCTTTTATGACTTTTGATGGTGAAGTCAAGACGGATGTG
AATAAGATCCAGGAGTTCTTAGAGGAGAAATTAGCTCCCCCGAGGTATCCCAAGCTGGGGACCCAACATCCC
GAATCTAATTCCGCAGGAAATGACGTGTTTGCCAAATTCTCAGCGTTTATAAAAAACACGAAGAAGGATGCA
AATGAGATTCATGAAAAGAACCTGCTGAAGGCCCTGAGGAAGCTGGATAATTACTTAAATAGCCCTCTGCCT
GATGAAATAGATGCCTACAGCACCGAGGATGTCACTGTTTCTGGAAGGAAGTTTCTGGATGGGGACGAGCTG
ACGCTGGCTGACTGCAACCTCTTACCCAAGCTCCATATTATTAAGATTGTGGCCAAGAAGTACAGAGATTTT
GAATTTCCTTCTGAAATGACTGGCATCTGGAGATACTTGAATAATGCTTATGCTAGAGATGAGTTCACAAAT
ACGTGTCCAGCTGATCAAGAGATTGAACACGCATATTCAGATGTTGCAAAAAGAATGAAATGAAGCT

In a search of public sequence databases, the NOV24b nucleic acid sequence, located on the q22.12 region of chromosome 21, has 741 of 847 bases (87%) identical to a parchorin mRNA from oryctolagus cuniculus gb accno AB035520.1) (E=3.2e⁻¹⁴⁰).

A disclosed NOV24b polypeptide (SEQ ID NO:106) encoded by SEQ ID NO:105 has 284 amino acid residues and is presented in Table 24D using the one-letter amino acid code. Signal P, Psort and/or Hydropathy results predict that NOV24b has no signal peptide and is likely to be localized to the nucleus with a certainty of 0.3000. Alternatively, NOV24b may also localize to the mitochondrial matrix space with a certainty of 0.1000, or to the lysosome (lumen) with a certainty of 0.1000.

TABLE 24D
Encoded NOV24b protein sequence.

(SEQ ID NO:106)

MAEAAEPEAQLSNHLAEEGPAEGSGEAARVNGRREDGEASEPRALGQEHDITLFVKAGYDGESIGNCPFSQR
LFMILWLKGVIFNVTTVDLKRKPADLQNLAPGTNPPFMTFDGEVKTDVNKIEEFLEEKLAPPRYPKLGTQHP
ESNSAGNDVFAKFSAFIKNTKKDANEIHEKNLLKALRKLDNYLNSPLPDEIDAYSTEDVTVSGRKFLDGDEL
TLADCNLLPKLHIIKIVAKKYRDFEFPSEMTGIWRYLNNAYARDEFTNTCPADQEIEHAYSDVAKRMK

A search of sequence databases reveals that the NOV24b amino acid sequence has 255 of 281 amino acid residues (90%) identical to, and 263 of 281 amino acid residues (93%) similar to, the 637 amino acid residue ptnr:SPTREMBL-ACC:Q9N2G5 protein from Oryctolagus cuniculus (Rabbit) (Parchorin) (E=1.6e⁻¹³⁴).

NOV24b disclosed in this invention is predicted to be expressed in at least the following tissues: heart, placent, skeletal muscle, stomach, and lung.

The disclosed NOV24a polypeptide has homology to the amino acid sequences shown in the BLASTP data listed in Table 24E.

TABLE 24E
BLAST results for NOV24a

| Gene Index/ | | Length | Identity | Positives | |
|---|---|---|---|---|---|
| Identifier | Protein/Organism | (aa) | (%) | (%) | Expect |
| gi\|7592636\|dbj\|BAA94345.1\| | parchorin | 637 | 436/715 | 475/715 | e−130 |
| (AB035520) | [Oryctolagus | | (60%) | (65%) | |
| | cuniculus] | | | | |
| gi\|6685319\|sp\|Q9Y696\| | CHLORIDE | 253 | 182/238 | 207/238 | e−108 |
| CLI4_HUMAN | INTRACELLULAR | | (76%) | (86%) | |
| | CHANNEL PROTEIN 4 | | | | |
| | (INTRACELLULAR | | | | |
| | CHLORIDE ION | | | | |
| | CHANNEL PROTEIN | | | | |
| | P64H1) | | | | |
| gi\|7330335\|ref\|NP_039234.1\| | chloride | 253 | 182/238 | 208/238 | e−108 |
| (NM_013943) | intracellular | | (76%) | (86%) | |
| | channel 4; | | | | |
| | chloride | | | | |
| | intracellular | | | | |
| | channel 4 like | | | | |
| | [Homo sapiens] | | | | |
| gi\|7304963\|ref\|NP_038913.1\| | chloride | 253 | 181/238 | 207/238 | e−107 |
| (NM_013885) | intracellular | | (76%) | (86%) | |
| | channel 4 | | | | |
| | (mitochondrial) | | | | |
| | [Mus musculus] | | | | |
| gi\|4588524\|gb\|AAD26136.1\| | intracellular | 253 | 180/238 | 205/238 | e−106 |
| AF109196_1 | chloride channel | | (75%) | (85%) | |
| (AF109196) | p64H1 [Homo | | | | |
| | sapiens] | | | | |

The homology between these and other sequences is shown graphically in the ClustalW analysis shown in Table 24F. In the ClustalW alignment of the NOV24 protein, as well as all other ClustalW analyses herein, the black outlined amino acid residues indicate regions of conserved sequence (i.e., regions that may be required to preserve structural or functional properties), whereas non-highlighted amino acid residues are less conserved and can potentially be altered to a much broader extent without altering protein structure or function.

[image]

[image]

[image]

The gene of invention encodes a homolog of parchorin, a new member of the intracellular chloride channel family. Parchorin was discovered as a 120 kDa phosphoprotein in gastric parietal cells (Urushidani et al., J Membr Biol. Apr. 1, 1999;168(3):209-20). Subsequent analysis revealed that this protein had significant homology to the family of intracellular chloride channels, especially in the C terminal domain (Nishizawa et al., J Biol Chem Apr. 14, 2000;275(15):11164-73). However, unlike other members of this family, parchorin exists mainly in the cytoplasm and translocated to the plasma membrane upon stimulation of chloride ion efflux. In addition, parchorin shows only two hydrophobic domains relative to the ten to twelve domains seen in other intracellular chloride channels. Tissue expression of parchorin in the rabbit is enhanced in cells that secrete water, like parietal cells, choroid plexus, salivary duct, lacrimal gland, kidney, airway epithelia, and chorioretinal epithelia. It is therefore thought that this protein plays a critical role in these tissues, possibly by modulating chloride ion transport.

Intracellular chloride channels have diverse roles within cells, such as volume regulation, acidification of intracellular vesicles, vectorial transepithelial chloride transport and regulation of cellular excitability (Jentsch et al., Pflugers Arch May 1999;437(6):783-95). Loss of function mutations affecting three different members of this family lead to three human inherited diseases: myotonia congenita, Dent's disease, and Bartter's syndrome. In addition, a mouse knockout model involving a member of this family has been generated that mimics diabetes insipidus (Matsumura et al., Nat Genet January 1999;21(1):95-8).

It is likely, therefore, that the protein of invention participates in physiological functions similar to those of other members of the intracellular chloride channel family, particularly parchorin.

The disclosed NOV24 nucleic acid of the invention encoding a Parchorin-like protein includes the nucleic acids whose sequences are provided in Table 24A or 24C or a fragment thereof. The invention also includes a mutant or variant nucleic acid any of whose bases may be changed from the corresponding base shown in Table 24A or 24C while still encoding a protein that maintains its Adrenal secretory serine protease-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 37 percent of the bases may be so changed.

The disclosed NOV24 protein of the invention includes the Parchorin-like protein whose sequence is provided in Table 24B or 24D. The invention also includes a mutant or variant protein any of whose residues may be changed from the corresponding residue shown in Table 24B or 24D while still encoding a protein that maintains its Adrenal secretory serine protease-like activities and physiological functions, or a functional fragment thereof. In the mutant or variant protein, up to about 40 percent of the residues may be so changed.

The invention further encompasses antibodies and antibody fragments, such as F_{ab} or (F_{ab})₂, that bind immunospecifically to any of the proteins of the invention.

The above disclosed information suggests that this Parchorin-like protein (NOV24) is a member of a “Parchorin family”. Therefore, the NOV24 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV24 nucleic acids and proteins of the invention are useful in potential therapeutic applications implicated in Von Hippel-Lindau (VHL) syndrome, Alzheimer's disease, stroke, tuberous sclerosis, hypercalceimia, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, neurodegeneration, systemic lupus erythematosus, autoimmune disease, asthma, emphysema, scleroderma, allergy, ARDS, diabetes, autoimmune disease, renal artery stenosis, interstitial nephritis, glomerulonephritis, polycystic kidney disease, systemic lupus erythematosus, renal tubular acidosis, IgA nephropathy, hypercalceimia, Lesch-Nyhan syndrome, cancer, trauma, bacterial/viral/parasitic infection, tissue degeneration, and/or other diseases and pathologies.

NOV24 nucleic acids and polypeptides are further useful in the generation of antibodies that bind immuno-specifically to the novel NOV24 substances for use in therapeutic or diagnostic methods. These antibodies may be generated according to methods known in the art, using prediction from hydrophobicity charts, as described in the “Anti-NOVX Antibodies” section below. The disclosed NOV24 protein has multiple hydrophilic regions, each of which can be used as an immunogen. These novel proteins can be used in assay systems for functional analysis of various human disorders, which will help in understanding of pathology of the disease and development of new drug targets for various disorders.

NOV25

A disclosed NOV25 nucleic acid of 1123 nucleotides (also referred to as CG56457-01) encoding a novel protein phosphatase-like protein is shown in Table 25A. An open reading frame was identified beginning with a ATG initiation codon at nucleotides 60-62 and ending with a TGA codon at nucleotides 768-770. The start and stop codons are shown in bold in Table 25A, and the 5′ and 3′ untranslated regions, if any, are underlined.

TABLE 25A
NOV25 nucleotide sequence.

(SEQ ID NO:107)

TCCGGATCGCTTCCCGGGCGGCGAGCTGGGGGTGCACCCGGACCGCCGCCCCCGGGATCATGGGCAATGGCA
TGACCAAGGTACTTCCTGGACTCTACCTCGGAAACTTCATTGATGCCAAAGACCTGGATCGCCTGGGCCGAA
ATAAGATCACACACATCATCTCTATCCATGAGTCACCCCAGCCTCTGCTGCAGGATATCACCTACCTTCGCA
TCCCGGTCGCTGATACCCCTGAGGTACCCATCAAAAAGCACTTCAAAGAATGTATCAACTTCATCCACTGCT
GCCGCCTTAATGGGGGGAACTGCCTTGTGCACTGCTTTGCAGGCATCTCTCGCAGCACCACGATTGTGACAG
CGTATGTGATGACTGTGACGGGGCTAGGCTGGCGGGACGTGCTTGAAGCCATCAAGGCCACCAGGCCCATCG
CCAACCCCAACCCAGGCTTTAGGCAGCAGCTTGAAGAGTTTGGCTGGGCCAGTTCCCAGAAGCTTCGCCGGC
AGCTGGAGGAGCGCTTCGGCGAGAGCCCCTTCCGCGACGAGGAGGACTTGCGCGCGCTGCTGCCTCTCTGCA
GGCGCTGTCGCCAGGGTCCGGGGACTTCGGCCCCGTCGGCCACCACAGCGTCCTCGGCCGCTTCCGAGGGGA
CCCTGCAGCGCCTGGTGCCGCGATCGCCGCGGGAATCACACCGGCCGCTGCCGCTGCTGGCGCGCGTCAAGC
AGACTTTCTCTTGCCTCCCCCGGTGTCTGTCCCGCAAGGGCGGCAAGTGAGGATGCAGTCCAGCCGTGGCTC
CCCACTTCCGACTGGCTCCCTTCGGGGGCTGTCTGCGCCTTCCACGCCCCCCAGGACGGGCCCAGAGGCTGG
GGGAGCCCCGCGGCCGCCTGAACCCTGCCTCCCGCGCCCGCCCTGCTCGTCCGCGTCTGCAGTCAGCGTCCC
CAACCTGTGCGTCTCTGTGTCCGGGCCGGCCTGCTGCAGCCACCTGGTGCCTTAGTCCTTGGGCTGGGGGAG
GGGGCCCACCCTTAAAGGCGGCCGGAGGGGAGGGAGGGAGAGTGGAGGGTTTGACGGGCCTGGAGGGTATTA
AAGAGACACAGAAGAAGCTGCCTGTCAAAAAAAAAAAAAAAAA

In a search of public sequence databases, the NOV25 nucleic acid sequence, located on chromosome 20, has 324 of 505 bases (64%) identical to a gb:GENBANK-ID:AF165519|acc:AF165519.1 mRNA from Homo sapiens (mitogen-activated protein kinase phosphatase x (MKPX) mRNA, complete cds) (E=2.3e⁻³¹).

A disclosed NOV25 polypeptide (SEQ ID NO:108) encoded by SEQ ID NO:107 has 236 amino acid residues and is presented in Table 25B using the one-letter amino acid code. Signal P, Psort and/or Hydropathy results predict that NOV25 has no signal peptide and is likely to be localized to the cytoplasm with a certainty of 0.6500. Alternatively, NOV25 may also localize to the lysosome (lumen) with a certainty of 0.1805, to the mitochondrial matrix space with a certainty of 0.1000, or to the plasma membrane with a certainty of 0.1000.

TABLE 25B
Encoded NOV25 protein sequence.

(SEQ ID NO:108)

MGNGMTKVLPCLYLGNFIDAKDLDRLGRNKITHIISIHESPQPLLQDITYLRIPVADTPEVPIKKHFKECIN
FIHCCRLNGGNCLVHCFAGISRSTTIVTAYVMTVTGLGWRDVLEAIKATRPIANPNPGFRQQLEEFGWASSQ
KLRRQLEERFGESPFRDEEDLRALLPLCRRCRQGPGTSAPSATTASSAASEGTLQRLVPRSPRESHRPLPLL
ARVKQTFSCLPRCLSRKGGK

A search of sequence databases reveals that the NOV25 amino acid sequence has 91 of 169 amino acid residues (53%) identical to, and 125 of 169 amino acid residues (73%) similar to, the 184 amino acid residue ptnr:SPTREMBL-ACC:Q9NRW4 protein from Homo sapiens (Human) (Mitogen-Activated Protein Kinase Phosphatase X) (E=7.3e⁻⁵⁰).

NOV25 is predicted to be expressed in at least brain, testis, exocrine pancreas, adipose, bone, peripheral blood, salivary glands, spinal cord, thyroid. This information was derived by determining the tissue sources of the sequences that were included in the invention including but not limited to SeqCalling sources, public EST sources and/or RACE sources.

In addition, the sequence is predicted to be expressed in hematopoietic stem cells because of the expression pattern of (GENBANK-ID: gb:GENBANK-ID:AF165519|acc:AF165519.1) a closely related Homo sapiens mitogen-activated protein kinase phosphatase x (MKPX) mRNA, complete cds homolog.

The disclosed NOV25 polypeptide has homology to the amino acid sequences shown in the BLASTP data listed in Table 25C.

TABLE 25C
BLAST results for NOV25

| Gene Index/ | | Length | Identity | Positives | |
|---|---|---|---|---|---|
| Identifier | Protein/Organism | (aa) | (%) | (%) | Expect |
| gi\|17458347\|ref\|XP_{—} | similar to | 235 | 223/236 | 229/236 | e−124 |
| 059288.1\| | bA243J16.6 (novel | | (94%) | (96%) | |
| (XM_059288) | protein with a | | | | |
| | dual specificity | | | | |
| | phosphatase, | | | | |
| | catalytic domain) | | | | |
| | (H. sapiens) | | | | |
| | [Homo sapiens] | | | | |
| gi\|18104942\|ref\|NP_{—} | dual specificity | 243 | 216/251 | 222/251 | e−115 |
| 542178.1\| | phosphatase-like | | (86%) | (88%) | |
| (NM_080611) | 15 [Homo sapiens] | | | | |
| gi\|9910432\|ref\|NP_064570.1\| | mitogen-activated | 184 | 91/169 | 125/169 | 4e−53 |
| (NM_020185) | protein kinase | | (53%) | (73%) | |
| | phosphatase x; | | | | |
| | homolog of mouse | | | | |
| | dual specificity | | | | |
| | phosphatase LMW- | | | | |
| | DSP2; JNK- | | | | |
| | stimulating | | | | |
| | phosphatase 1 | | | | |
| | [Homo sapiens] | | | | |
| gi\|13183069\|gb\|AAK15038.1\| | dual specificity | 184 | 90/169 | 125/169 | 2e−52 |
| AF237619_1 | phosphatase TS- | | (53%) | (73%) | |
| (AF237619) | DSP2 [Mus | | | | |
| | musculus] | | | | |
| gi\|14726046\|ref\|XP_{—} | mitogen-activated | 184 | 89/169 | 118/169 | 2e−50 |
| 046543.1\| | protein kinase | | (52%) | (69%) | |
| (XM_046543) | phosphatase x | | | | |
| | [Homo sapiens] | | | | |

The homology between these and other sequences is shown graphically in the ClustalW analysis shown in Table 25D. In the ClustalW alignment of the NOV25 protein, as well as all other ClustalW analyses herein, the black outlined amino acid residues indicate regions of conserved sequence (i.e., regions that may be required to preserve structural or functional properties), whereas non-highlighted amino acid residues are less conserved and can potentially be altered to a much broader extent without altering protein structure or function.

[image]

[image]

Tables 25E-H list the domain descriptions from DOMAIN analysis results against NOV25. This indicates that the NOV25 sequence has properties similar to those of other proteins known to contain this domain.

TABLE 25E

Domain Analysis of NOV25

gn1|Smart|smart00195, DSPc, Dual specificity phosphatase, catalytic
domain (SEQ ID NO:816)
CD-Length = 139 residues, 97.8% aligned
Score = 139 bits (349), Expect = 2e−34

| | | | |
|---|---|---|---|
| NOV25: | 4 | GMTKVLPGLYLGNFIDAKDLDRLGRNKITHIIS-IHESPQPLLQDITYLRIPVADTPEVP | 62 |
| | | \| +++\|\| \|\|\|\|++ \|\| +\| \| + \|\|\|+\|+ \| \| \|\| \|\|\| \| \| | |
| Sbjct: | 1 | GPSEILPHLYLGSYSDASNLALLKKLGITHVINVTEEVPNSNKSGFLYLGIPVDDNTETK | 60 |
| NOV25: | 63 | IKKHFKECINFIHCCRLNGGNCLVHCFAGISRSTTIVTAYVMTVTGLGWRDVLEAIKATR | 122 |
| | | \| + \| + \|\| \|\| \|\|\|\| \|\|+\|\|\| \|++ \|\|+\| + \| + +\| \| | |
| Sbjct: | 61 | ISPYLPEAVEFIEDAEKKGGKVLVHCQAGVSRSATLIIAYLMKYRNMSLNDAYDFVKERR | 120 |
| NOV25: | 123 | PIANPNPGFRQQLEEF 138 | |
| | | \|\| +\|\| \|\| +\|\| \|+ | |
| Sbjct: | 121 | PIISPNFGFLRQLIEY 136 | |

TABLE 25F

Domain Analysis of NOV25

gn1|Pfam|pfam00782, DSPc, Dual specificity phosphatase, catalytic
domain. Ser/Thr and Tyr protein phosphatases. The enzyme's tertiary
fold is highly similar to that of tyrosine-specific phosphatases,
except for a “recognition” region. (SEQ ID NO:817)
CD-Length = 139 residues, 97.8% aligned
Score = 136 bits (342), Expect = 2e−33

| | | | |
|---|---|---|---|
| NOV25: | 4 | GMTKVLPGLYLGNFIDAKDLDRLCRNKITHIIS-IHESPQPLLQDITYLRIPVADTPEVP | 62 |
| | | \| +++\|\| \|\|\|\|++ \| +\| \| + \|\|\|+\|+ \| \| \|\| \|\|\| \| \| | |
| Sbjct: | 1 | GPSEILPHLYLGSYPTASNLAFLSKLGITHVINVTEEVPNSKNSCFLYLHIPVDDNHETD | 60 |
| NOV25: | 63 | IKKHFKECINFIHCCRLNGGNCLVHCFAGISRSTTIVTAYVMTVTGLGWRDVLEAIKATR | 122 |
| | | \| + \| + \|\| \| \|\| \|\|\|\| \|\|\|\|\|\| \|++ \|\|+\| \| + +\| \| | |
| Sbjct: | 61 | ISPYLDEAVEFIEDARQKGGKVLVHCQAGISRSATLIIAYLMKTRNLSLNEAYSFVKERR | 120 |
| NOV25: | 123 | PIANPNPGFRQQLEEF 138 | |
| | | \|\| +\|\| \|\|++\|\| \|+ | |
| Sbjct: | 121 | PIISPNFGFKRQLIEY 136 | |

TABLE 25G

Domain Analysis of NOV25

gn1|Smart|smart00404, PTPc_motif, Protein tyrosine phosphatase,
catalytic domain motif (SEQ ID NO:818)
CD-Length = 105 residues, 53.3% aligned
Score = 41.2 bits (95), Expect = 7e−05

| | | | |
|---|---|---|---|
| NOV25: | 50 | YLRIPVADTPEVPIK-KHFKECINFIHCCRLNGGNCLVHCFAGISRSTTIVTAYVM | 104 |
| | | \| \| \|\| \| \| + \| \| +\|\|\| \|\|+ \|+ \| \| ++ | |
| Sbjct: | 7 | YTGWPDHGVPESPDSILEFLPAVKKSLNKSANNGPVVVHCSAGVGRTGTFVAIDIL | 62 |

TABLE 25H

Domain Analysis of NOV25

gn1|Pfam|pfam00102, Y_phosphatase, Protein-tyrosine phosphatase
(SEQ ID NO:819)
CD-Length = 1235 residues, 31.9% aligned
Score = 138.5 bits (88), Expect = 14e−04

| | | | |
|---|---|---|---|
| NOV25: | 50 | YLRIPVADTPEVPIKKHFKECINFIHCCRLNG--GNCLVHCFAGISRSTTIVTAYVM--T | 105 |
| | | \| \| \|\| \| \| + + + + \| +\|\|\| \|\|\| \|+ \| + ++ | |
| Sbjct: | 139 | YTGWPDHGVPESP--KSILDLLRKVRKSKGTPDDGPIVVHCSAGIGRTGTFIAIDILLQQ | 196 |
| NOV25: | 106 | VTGLGWRDVLEAIKATR 122 | |
| | | + \| \|\| + +\| \| | |
| Sbjct: | 197 | LEKEGVVDVFDTVKKLR 213 | |

The gene of invention is a member of the family of dual specificity protein phosphatases (DSPs; Martell et al., Mol Cells Feb. 28, 1998;8(1):2-11). DSPs recognize either Ser/Thr or Tyr moieties as targets for dephosphorylation. These enzymes regulate mitogenic signal transduction and can thereby regulate the cell cycle. Some members of this family are effective tumor suppressors, for example, PTEN. PTEN is required during embryonic development and later in life, and mutations in this gene give rise to different kinds of inherited and sporadic cancers (Eng, Recent Prog Horm Res 1999;54:441-52; discussion 453). In Drosophila, members of the DSP family, such as puckered, have important roles in development (Martin-Blanco et al., Genes Dev Feb. 15, 1998;12(4):557-70). The crystal structure of one member of the DSP family has been elucidated (Yuvaniyama at al., Science May 31, 1996;272(5266):1328-31) and this family has been successfully targeted for small molecule drug development (Ducruet et al., Bioorg Med Chem June 2000;8(6):1451-66). In addition, overexpression of a DSP has been demonstrated to be a potential therapy for cardiac hypertrophy (Bueno et al., Circ Res Jan. 19, 2001;88(1):88-96). The gene of invention has greatest homology to a DSP identified in hematopoietic stem/progenitor cells from a patient with myelodysplastic syndromes. It shows the presence of a distinct domain present in all DSPs, which qualifies it as a bona fide member of this family. Its localization is predicted to be cytoplasmic, which makes it a good candidate to interact with members of the signal transduction cascade governing the cell cycle.

The disclosed NOV25 nucleic acid of the invention encoding a Protein phosphatase-like protein includes the nucleic acid whose sequence is provided in Table 25A or a fragment thereof. The invention also includes a mutant or variant nucleic acid any of whose bases may be changed from the corresponding base shown in Table 25A while still encoding a protein that maintains its Protein phosphatase-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 36 percent of the bases may be so changed.

The disclosed NOV25 protein of the invention includes the Protein phosphatase-like protein whose sequence is provided in Table 25B. The invention also includes a mutant or variant protein any of whose residues may be changed from the corresponding residue shown in Table 25B while still encoding a protein that maintains its Protein phosphatase-like activities and physiological functions, or a functional fragment thereof. In the mutant or variant protein, up to about 48 percent of the residues may be so changed.

The invention further encompasses antibodies and antibody fragments, such as F_{ab} or (F_{ab})₂, that bind immunospecifically to any of the proteins of the invention.

The above disclosed information suggests that this Protein phosphatase-like protein (NOV25) is a member of a “Protein phosphatase family”. Therefore, the NOV25 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV25 nucleic acids and proteins of the invention are useful in potential therapeutic applications implicated in for example Von Hippel-Lindau (VHL) syndrome, pancreatitis, obesity, Alzheimer's disease, stroke, tuberous sclerosis, hypercalceimia, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, neurodegeneration, psychiatric disorders, metabolic disorders, fertility, hypogonadism, xerostomia, hyperthyroidism, hypothyroidism, cancer, trauma, tissue degeneration, viral/bacterial/parasitic infections, and/or other diseases and pathologies.

NOV25 nucleic acids and polypeptides are further useful in the generation of antibodies that bind immuno-specifically to the novel NOV25 substances for use in therapeutic or diagnostic methods. These antibodies may be generated according to methods known in the art, using prediction from hydrophobicity charts, as described in the “Anti-NOVX Antibodies” section below. The disclosed NOV25 protein has multiple hydrophilic regions, each of which can be used as an immunogen. These novel proteins can be used in assay systems for functional analysis of various human disorders, which will help in understanding of pathology of the disease and development of new drug targets for various disorders.

NOV26

NOV26 includes two novel GAGE-7-like proteins disclosed below. The disclosed sequences have been named NOV26a and NOV26b.

NOV26a

A disclosed NOV26a nucleic acid of 550 nucleotides (also referred to as CG56461-01) encoding a novel GAGE-7-like protein is shown in Table 26A. An open reading frame was identified beginning with a ATG initiation codon at nucleotides 67-69 and ending with a TAA codon at nucleotides 400-402. The start and stop codons are shown in bold in Table 26A, and the 5′ and 3′ untranslated regions, if any, are underlined.

TABLE 26A

NOV26a nucleotide sequence.

(SEQ ID NO:109)

GTTCCTGCTGTCTGGACTTTTTCTGTCCCACTGAGACGCAGCTGTATTCTGTTTGCAGTGTGAAATATGATT
TGGCGAGGAAGATCAACATATAGGCCTAGGCCGAGGAGAAGTGTACCACCTCCTGAGCTGATTGGGCCTATG
CTGGAGCCCGGTGATGAGGAGCCTCAGCAAGAGGAACCCCCAACTGAAAGTCGGGATCCTGCACCTGGTCAG
GAGAGAGAAGAAGATCAGGGTGCAGCTGAGACTCAAGTGCCTGACCTGGAAGCTGATCTCCAGGAGCTGTCT
CAGTCAAAGACTGGGGGTGAATGTGGAAATGGTCCTGATGACCAGGGGAAGATTCTGCCAAAATCAGAACAA
TTTAAAATGCCAGAAGGAGGTGACAGGCAACCACAGGTTTAAATGAAGACAAGCTGAAACAACACAAAACTG
TTTTTATCTAAGATATTTGACTTAAAAATATCGAATAAACTTTTGCAGCTTTCTCCAAAAAAAAAAAAAAAA
AAAAAAAAAAAAAAAAAAAAAAAAAAAAAAAAAAAAAAACCCCGC

In a search of public sequence databases, the NOV26a nucleic acid sequence, located on the X chromosome, has 293 of 360 bases (81%) identical to a gb:GENBANK-ID:AF251237|acc:AF251237.1 mRNA from Homo sapiens (XAGE-1 mRNA, complete cds) (E=3.6e⁻⁴⁶).

A disclosed NOV26a polypeptide (SEQ ID NO:110) encoded by SEQ ID NO:109 has 111 amino acid residues and is presented in Table 26B using the one-letter amino acid code. Signal P, Psort and/or Hydropathy results predict that NOV26a has no signal peptide and is likely to be localized to the mitochondrial matrix space with a certainty of 0.4462. Alternatively, NOV26A may also localize to the nucleuswith a certainty of 0.3000, to the mitochondrial inner membrane with a certainty of 0.1347, or to the mitochondrial intermembrane space with a certainty of 0.1347.

TABLE 26B

Encoded NOV26a protein sequence.

(SEQ ID NO:110)

MIWRGRSTYRPRPRRSVPPPELIGPMLEPGDEEPQQEEPPTESRDPAPGQ
EREEDQGAAETQVPDLEADLQELSQSKTGGECGNGPDDQGKILPKSEQFK
MPEGGDRQPQV

A search of sequence databases reveals that the NOV26a amino acid sequence has 60 of 115 amino acid residues (52%) identical to, and 72 of 115 amino acid residues (62%) similar to, the 116 amino acid residue ptnr:SPTREMBL-ACC:Q9UEU5 protein from Homo sapiens (Human) (GAGE-7) (E=1.4e⁻²³).

NOV26a is predicted to be expressed in at least placenta. This information was derived by determining the tissue sources of the sequences that were included in the invention including but not limited to SeqCalling sources, Public EST sources, and/or RACE sources.

NOV26b

In the present invention, the target sequence identified previously, NOV26a, was subjected to the exon linking process to confirm the sequence. PCR primers were designed by starting at the most upstream sequence available, for the forward primer, and at the most downstream sequence available for the reverse primer. In each case, the sequence was examined, walking inward from the respective termini toward the coding sequence, until a suitable sequence that is either unique or highly selective was encountered, or, in the case of the reverse primer, until the stop codon was reached. Such primers were designed based on in silico predictions for the full length cDNA, part (one or more exons) of the DNA or protein sequence of the target sequence, or by translated homology of the predicted exons to closely related human sequences sequences from other species. These primers were then employed in PCR amplification based on the following pool of human cDNAs: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea, uterus. Usually the resulting amplicons were gel purified, cloned and sequenced to high redundancy. The resulting sequences from all clones were assembled with themselves, with other fragments in CuraGen Corporation's database and with public ESTs. Fragments and ESTs were included as components for an assembly when the extent of their identity with another component of the assembly was at least 95% over 50 bp. In addition, sequence traces were evaluated manually and edited for corrections if appropriate. These procedures provide the sequence reported below, which is designated NOV26b. This is 100% identical to the previously identified sequence (NOV26a).

A disclosed NOV26b nucleic acid of 494 nucleotides (also referred to as CG56461-02) encoding a novel GAGE-7-like protein is shown in Table 26C. An open reading frame was identified beginning with a ATG initiation codon at nucleotides 67-69 and ending with a TAA codon at nucleotides 400-402. The start and stop codons are shown in bold in Table 26C, and the 5′ and 3′ untranslated regions, if any, are underlined.

TABLE 25C
NOV26b nucleotide sequence.

(SEQ ID NO:111)

GTTCCTGCTGTCTGGACTTTTTCTGTCCCACTGAGACGCAGCTGTATTCTGTTTGCAGTGTGAAATATGATT
TGGCGAGGAAGATCAACATATAGGCCTAGGCCGAGGAGAAGTGTACCACCTCCTGAGCTGATTGGGCCTATG
CTGGAGCCCGGTGATGAGGAGCCTCAGCAAGAGGAACCACCAACTGAAAGTCGGGATCCTGCACCTGGTCAG
GAGAGAGAAGAAGATCAGGGTGCAGCTGAGACTCAAGTGCCTGACCTGGAAGCTGATCTCCAGGAGCTGTCT
CAGTCAAAGACTGGGGGTGAATGTGGAAATGGTCCTGATGACCAGGGGAAGATTCTGCCAAAATCAGAACAA
TTTAAAATGCCAGAAGGAGGTGACAGGCAACCACAGGTTTAAATGAAGACAAGCTGAAACAACACAAAACTG
TTTTTATCTAAGATATTTGACTTAAAAATATCAAAATAAACTTTTGCAGCTTTCTCCAAAAA

In a search of public sequence databases, the NOV26b nucleic acid sequence, located on the X chromosome, has 346 of 426 bases (81%) identical to a gb:GENBANK-ID:HSL185E6A|acc:Z68274.1 mRNA from Homo sapiens (Human DNA sequence from cosmid L129H7, Huntington's Disease Region, chromosome 4p16.3 contains Pseudogene and CpG island) (E=5.7e⁻⁵³).

The disclosed NOV26b polypeptide (SEQ ID NO:112) encoded by SEQ ID NO:111 has 111 amino acid residues and is presented in Table 26D using the one-letter amino acid code. Signal P, Psort and/or Hydropathy results predict that NOV26b has no signal peptide and is likely to be localized to the mitochondrial matrix space with a certainty of 0.4462. Alternatively, NOV26b may also localize to the nucleus with a certainty of 0.3000, to the mitochondrial inner membrane with a certainty of 0.1347, or to the mitochondrial intermembrane space with a certainty of 0.1347.

TABLE 26D

Encoded NOV26b protein sequence.

(SEQ ID NO:112)

MIWRGRSTYRPRPRRSVPPPELIGPMLEPGDEEPQQEEPPTESRDPAPGQ
EREEDQGAAETQVPDLEADLQELSQSKTGGECGNGPDDQGKILPKSEQFK
MPEGGDRQPQV

A search of sequence databases reveals that the NOV26b amino acid sequence has 60 of 115 amino acid residues (52%) identical to, and 72 of 115 amino acid residues (62%) similar to, the 116 amino acid residue ptnr:SPTREMBL-ACC:Q9UEU5 protein from Homo sapiens (Human) (GAGE-7) (E=1.4e⁻²³).

NOV26b is predicted to be expressed in at least the following tissues: Placenta, Whole Organism.

The disclosed NOV26a polypeptide has homology to the amino acid sequences shown in the BLASTP data listed in Table 26E.

TABLE 26E
BLAST results for NOV26a

| Gene Index | | Length | Identity | Positives | |
|---|---|---|---|---|---|
| Identifier | Protein/Organism | (aa) | (%) | (%) | Expect |
| gi\|17486397\|ref\|XP_{—} | similar to G | 137 | 84/84 | 84/84 | 2e−33 |
| 060048.1\| | antigen 3 (H. | | (100%) | (100%) | |
| (XM_060048) | sapiens) [Homo | | | | |
| | sapiens] | | | | |
| gi\|18027836\|gb\|AAL55879.1\| | unknown [Homo | 111 | 110/111 | 110/111 | 4e−33 |
| AF318372_1 | sapiens] | | (99%) | (99%) | |
| (AF318372) | | | | | |
| gi\|18157212\|emb\|CAC83008.1\| | XAGE-3 protein | 111 | 111/111 | 111/111 | 2e−29 |
| (AJ318881) | [Homo sapiens] | | (100%) | (100%) | |
| gi\|17486394\|ref\|XP_{—} | similar to G | 185 | 64/78 | 69/78 | 2e−26 |
| 066835.1\| | antigen B1; | | (82%) | (88%) | |
| (XM_066835) | prostate | | | | |
| | associated gene 1 | | | | |
| | (H. sapiens) | | | | |
| | [Homo sapiens] | | | | |
| gi\|14765261\|ref\|XP_{—} | hypothetical | 111 | 80/111 | 93/111 | 4e−26 |
| 032309.1\| | protein XP_032309 | | (72%) | (83%) | |
| (XM_032309) | [Homo sapiens] | | | | |

The homology between these and other sequences is shown graphically in the ClustalW analysis shown in Table 26F. In the ClustalW alignment of the NOV26 protein, as well as all other ClustalW analyses herein, the black outlined amino acid residues indicate regions of conserved sequence (i.e., regions that may be required to preserve structural or functional properties), whereas non-highlighted amino acid residues are less conserved and can potentially be altered to a much broader extent without altering protein structure or function.

[image]

[image]

The gene of invention is a member of GAGE family of proteins. It belongs to the broad family of GAGE/MAGE/PAGE genes that are expressed in various human cancers. Many human tumors express antigens that are recognized in vitro by cytolyticT lymphocytes (CTLs) derived from the tumor-bearing patient. The MAGE(melanoma-specific antigen), PAGE (Prostate cancer antigen) and GAGE (G antigen) gene family members encode such antigens. Therefore these antigens can serve as therapeutic targets in cancer.

The LNCaP progression model of human prostate cancer consists of lineage-related sublines that differ in their androgen sensitivity and metastatic potential. A differential display polymerase chain reaction was employed by Chen ME, et al. (J Biol Chem Jul. 10, 1998;273(28):17618-25) to evaluate mRNA expression differences between the LNCaP sublines in order to define the differences in gene expression between the androgen-sensitive, nontumorigenic LNCaP cell line and the androgen-insensitive, metastatic LNCaP sublines, C4-2 and C4-2B. An amplicon, BG16.21, was isolated that showed increased expression in the androgen-independent and metastatic LNCaP sublines, C4-2 and C4-2B. Hybridization screening of a lambda gt11 expression library with BG16.21 revealed two transcripts, both homologous to BG16.21 at the 3′ end. A GenBank™ data base search using the GCG Wisconsin software package revealed the shorter approximately 600-bp transcript (designated GAGE-7) to be a new member of the GAGE family. The second approximately 700-bp transcript was a novel gene (designated PAGE-1, “prostate associated gene”) with only 45% homology to GAGE gene family members. RNA blot analysis demonstrated that GAGE-7 mRNA was expressed at equal levels in all lineage related prostate cancer cell sublines, while PAGE-1 mRNA levels were elevated 5-fold in C4-2 and C4-2B as compared with LNCaP cells. Neither GAGE-7 nor PAGE-1 demonstrated any regulation by androgens in the prostate cancer cell lines used in this study. PAGE-1 and GAGE-7 expression was found to be restricted to testes (high) and placenta (low) on human multiple tissue Northern blots. As GAGE/MAGE antigens were reported previously to be targets for tumor-specific cytotoxic lymphocytes in melanoma, these results suggest that PAGE-1 and GAGE-7 may be related to prostate cancer progression and may serve as potential targets for novel therapies.

The GAGE-1 gene was identified previously as a gene that codes for an antigenic peptide, YRPRPRRY, which was presented on a human melanoma by HLA-Cw6 molecules and recognized by a clone of CTLs derived from the patient bearing the tumor. By screening a cDNA library from this melanoma, De Backer O, et al. (Cancer Res Jul. 1, 1999;59(13):3157-65) identified five additional, closely related genes named GAGE-2-6. We report here that further screening of this library led to the identification of two more genes, GAGE-7B and -8. GAGE-1, -2, and -8 code for peptide YRPRPRRY. Using another antitumor CTL clone isolated from the same melanoma patient, they identified antigenic peptide, YYWPRPRRY, which is encoded by GAGE-3, -4, -5, -6, and -7B and which is presented by HLA-A29 molecules. Genomic cloning of GAGE-7B showed that it is composed of five exons. Sequence alignment showed that an additional exon, which is present only in the mRNA of GAGE-1, has been disrupted in gene GAGE-7B by the insertion of a long interspersed repeated element retroposon. These GAGE genes are located in the p11.2-p11.4 region of chromosome X. They are not expressed in normal tissues, except in testis, but a large proportion of tumors of various histological origins express at least one of these genes. Treatment of normal and tumor cultured cells with a demethylating agent, azadeoxycytidine, resulted in the transcriptional activation of GAGE genes, suggesting that their expression in tumors results from a demethylation process.

The disclosed NOV26 nucleic acid of the invention encoding a GAGE-7-like protein includes the nucleic acid whose sequence is provided in Table 26A, 26C or a fragment thereof. The invention also includes a mutant or variant nucleic acid any of whose bases may be changed from the corresponding base shown in Table 26A or 26C while still encoding a protein that maintains its GAGE-7-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 19 percent of the bases may be so changed.

The disclosed NOV26 protein of the invention includes the GAGE-7-like protein whose sequence is provided in Table 26B or 26D. The invention also includes a mutant or variant protein any of whose residues may be changed from the corresponding residue shown in Table 26B or 26D while still encoding a protein that maintains its GAGE-7-like activities and physiological functions, or a functional fragment thereof. In the mutant or variant protein, up to about 28 percent of the residues may be so changed.

The invention further encompasses antibodies and antibody fragments, such as F_{ab} or (F_{ab})₂, that bind immunospecifically to any of the proteins of the invention.

The above disclosed information suggests that this GAGE-7-like protein (NOV26) is a member of a “GAGE-7 family”. Therefore, the NOV26 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV26 nucleic acids and proteins of the invention are useful in potential therapeutic applications implicated in fertility disorders, cancer, trauma, tissue degeneration, viral/bacterial/parasitic infections, and/or other diseases and pathologies.

NOV26 nucleic acids and polypeptides are further useful in the generation of antibodies that bind immuno-specifically to the novel NOV26 substances for use in therapeutic or diagnostic methods. These antibodies may be generated according to methods known in the art, using prediction from hydrophobicity charts, as described in the “Anti-NOVX Antibodies” section below. The disclosed NOV26 protein has multiple hydrophilic regions, each of which can be used as an immunogen. These novel proteins can be used in assay systems for functional analysis of various human disorders, which will help in understanding of pathology of the disease and development of new drug targets for various disorders.

NOV27

NOV27 includes three novel sodium-glucose cotransporter-like proteins disclosed below. The disclosed sequences have been named NOV27a, NOV27b, and NOV27c.

NOV27a

A disclosed NOV27a nucleic acid of 1914 nucleotides (also referred to as CG56645-01) encoding a novel sodium-glucose cotransporter-like protein is shown in Table 27A. An open reading frame was identified beginning with a ATG initiation codon at nucleotides 51-53 and ending with a TGA codon at nucleotides 1839-1841. The start and stop codons are shown in bold in Table 27A, and the 5′ and 3′ untranslated regions, if any, are underlined.

TABLE 27A

NOV27a nucleotide sequence.

(SEQ ID NO:113)

TTGCCCCTCAGTCCCTCGGGCTCATACCTAGTGCCTGCGGCAGGACAGCCATGGCCGCCAACTCCACCAGCG
ACCTCCACACTCCCGGGACGCAGCTGAGCGTGGCTGACATCATCGTCATCACTGTGTATTTTGCTCTGAACG
TGGCCGTGGGCATATGGTCCTCTTGTCGGGCCAGTAGGAACACGGTGAATGGCTACTTCCTGGCAGGCCGGG
ACATGACGTGGTGGCCGATTGGAGCCTCCCTCTTCGCCAGCAGCGAGGGCTCTGGCCTCTTCATTGGACTGG
CGGGCTCAGGCGCGGCAGGAGGTCTGGCCGTGGCAGGCTTCGAGTGGAATGCCACGTACGTGCTGCTGGCAC
TGGCATGGGTGTTCGTGCCCATCTACATCTCCTCAGAGATCGTCACCTTACCTGAGTACATTCAGAAGCGCT
ACGGGGGCCAGCGGATCCGCATGTACCTGTCTGTCCTGTCCCTGCTACTGTCTGTCTTCACCAAGATATCGC
TGGACCTGTACGCGGGGGCTCTGTTTGTGCACATCTGCCTGGGCTGGAACTTCTACCTCTCCACCATCCTCA
CGCTCGGCATCACAGCCCTGTACACCATCGCAGGGGGCCTGGCTGCTGTAATCTACACGGACGCCCTGCAGA
CGCTCATCATGGTGGTGGGGCCTGTCATCCTGACAATCAAAGCTTTTGACCAGATCGGTGGTTACGGGCAGC
TGGAGGCAGCCTACGCCCAGGCCATTCCCTCCAGGACCATTGCCAACACCACCTGCCACCTGCCACGTACAG
ACGCCATGCACATGTTTCGAGACCCCCACACAGGGGACCTGCCGTGGACCGGGATGACCTTTGGCCTGACCA
TCATGGCCACCTGGTACTGGTGCACCGACCAGGTGATCGTGCAGCGATCACTGTCAGCCCGGGACCTGAACC
ATGCCAAGGCGGGCTCCATCCTGGCCAGCTACCTCAAGATGCTCCCCATGGGCCTGATCATAATGCCGGGCA
TGATCAGCCGCGCATTGTTCCCAGATGATGTGGGCTGCGTGGTGCCGTCCGAGTGCCTGCGGGCCTGCGGGG
CCGAGGTCGGCTGCTCCAACATCGCCTACCCCAAGCTGGTCATGGAACTGATGCCCATCGGTCTGCGGGGGC
TGATGATCGCAGTGATGCTGGCGGCGCTCATGTCGTCGCTGACCTCCATCTTCAACAGCAGCAGCACCCTCT
TCACTATGGACATCTGGAGGCGGCTGCGTCCCCGCTCCGGCGAGCGGGAGCTCCTGCTGGTGGGACGGCTGG
TCATAGTGGCACTCATCGGCGTGAGTGTGGCCTGGATCCCCGTCCTGCAGGACTCCAACAGCGGGCAACTCT
TCATCTACATGCAGTCAGTGACCAGCTCCCTGGCCCCACCAGTGACTGCAGTCTTTGTCCTGGGCGTCTTCT
GGCGACGTGCCAACGAGCAGGGGGCCTTCTGGGGCCTGATAGCAGGGCTGGTGGTGGGGGCCACGAGGCTGG
TCCTGGAATTCCTGAACCCAGCCCCACCGTGCGGAGAGCCAGACACGCGGCCAGCCGTCCTGGGGAGCATCC
ACTACCTGCACTTCGCTGTCGCCCTCTTTGCACTCAGTGGTGCTGTTGTGGTGGCTGGAAGCCTGCTGACCC
CACCCCCACAGAGTGTCCAGATTGAGAACCTTACCTGGTGGACCCTGGCTCAGGATGTGCCCTTGGGAACTA
AAGCAGGTGATGGCCAAACACCCCAGAAACACGCCTTCTGGGCCCGTGTCTGTGGCTTCAATGCCATCCTCC
TCATGTGTOTCAACATATTCTTTTATGCCTACTTCGCCTGACACTGCCATCCTGGACAGAAAGGCAGGAGCT
CTGAGTCCTCAGGTCCACCCATTTCCCTCATGGGGATCCCGA

In a search of public sequence databases, the NOV27a nucleic acid sequence, located on chromosome 17, has 1598 of 1838 bases (86%) identical to a gb:GENBANK-ID:OCU08813|acc:U08813.1 mRNA from Oryctolagus cuniculus (Oryctolagus cuniculus Na+/glucose cotransporter-related protein mRNA, complete cds) (E=2.6e⁻³⁰⁹).

A disclosed NOV27a polypeptide (SEQ ID NO:114) encoded by SEQ ID NO:113 has 596 amino acid residues and is presented in Table 27B using the one-letter amino acid code. Signal P, Psort and/or Hydropathy results predict that NOV27A has a signal peptide and is likely to be localized to the plasma membrane with a certainty of 0.8200. Alternatively, NOV27a may also localize to the endoplasmic reticulum (membrane) with a certainty of 0.6850, to the Golgi body with a certainty of 0.4600, or to the enoplasmic reticulum (lumen) with a certainty of 0.1000. The most likely cleavage site for NOV27A is between positions 42 and 43: CRA-SR.

TABLE 27B
Encoded NOV27a protein sequence.

(SEQ ID NO:114)

MAANSTSDLHTPGTQLSVADIIVITVYFALNVAVGIWSSCRASRNTVNGYFLAGRDMTWWPIGASLFASSEG
SGLFIGLAGSGAAGGLAVAGFEWNATYVLLALAWVFVPIYISSEIVTLPEYIQKRYGGQRIRMYLSVLSLLL
SVFTKISLDLYAGALFVHICLGWNFYLSTILTLGITALYTIAGGLAAVIYTDALQTLIMVVGAVILTIKAFD
QIGGYGQLEAAYAQAIPSRTIANTTCHLPRTDAMHMFRDPHTGDLPWTGMTFGLTIMATWYWCTDQVIVQRS
LSARDLNHAKAGSILASYLKMLPMGLIIMPGMISRALFPDDVGCVVPSECLRACGAEVGCSNIAYPKLVMEL
MPIGLRGLMIAVMLAALMSSLTSIFNSSSTLFTMDIWRRLRPRSGERELLLVGRLVIVALIGVSVAWIPVLQ
DSNSGQLFIYMQSVTSSLAPPVTAVFVLGVFWRRANEQGAFWGLIAGLVVGATRLVLEFLNPAPPCGEPDTR
PAVLGSIHYLHFAVALFALSGAVVVAGSLLTPPPQSVQIENLTWWTLAQDVPLGTKAGDGQTPQKHAFWARV
CGFNAILLMCVNIFFYAYFA

A search of sequence databases reveals that the NOV27a amino acid sequence has 531 of 596 amino acid residues (89%) identical to, and 559 of 596 amino acid residues (93%) similar to, the 597 amino acid residue ptnr:SPTREMBL-ACC:Q28610 protein from Oryctolagus cuniculus (Rabbit) (Na+/Glucose Cotransporter-Related Protein) (E=1.1e⁻²⁸⁹).

NOV27a is predicted to be expressed in at least heart and kidney. This information was derived by determining the tissue sources of the sequences that were included in the invention including but not limited to SeqCalling sources, public EST sources, and/or RACE sources.

In addition, the sequence is predicted to be expressed in the following tissues because of the expression pattern of (GENBANK-ID: gb:GENBANK-ID:OCU08813|acc:U08813.1) a closely related Oryctolagus cuniculus Na+/glucose cotransporter-related protein mRNA, complete cds homolog.

NOV27b

In the present invention, the target sequence identified previously, NOV27a, was subjected to the exon linking process to confirm the sequence. PCR primers were designed by starting at the most upstream sequence available, for the forward primer, and at the most downstream sequence available for the reverse primer. In each case, the sequence was examined, walking inward from the respective termini toward the coding sequence, until a suitable sequence that is either unique or highly selective was encountered, or, in the case of the reverse primer, until the stop codon was reached. Such primers were designed based on in silico predictions for the full length cDNA, part (one or more exons) of the DNA or protein sequence of the target sequence, or by translated homology of the predicted exons to closely related human sequences sequences from other species. These primers were then employed in PCR amplification based on the following pool of human cDNAs: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea, uterus. Usually the resulting amplicons were gel purified, cloned and sequenced to high redundancy. The resulting sequences from all clones were assembled with themselves, with other fragments in CuraGen Corporation's database and with public ESTs. Fragments and ESTs were included as components for an assembly when the extent of their identity with another component of the assembly was at least 95% over 50 bp. In addition, sequence traces were evaluated manually and edited for corrections if appropriate. These procedures provide the sequence reported below, which is designated NOV27b. This differs from the previously identified sequence (NOV27a) in having 16 extra internal amino acids.

A disclosed NOV27b nucleic acid of 1912 nucleotides (also referred to as CG56645-02 encoding a novel sodium-glucose cotransporter-like protein is shown in Table 27C. An open reading frame was identified beginning with a ATG initiation codon at nucleotides 35-37 and ending with a TGA codon at nucleotides 1871-1873. The start and stop codons are shown in bold in Table 27C, and the 5′ and 3′ untranslated regions, if any, are underlined.

TABLE 27C
NOV27b nucleotide sequence.

(SEQ ID NO:115)

CGGGCTCATACCTAGTGCCTGCGGCAGGACAGCCATGGCCGCCAACTCCACCAGCGACCTCCACACTCCCGG
GACGCAGCTGAGCGTGGCTGACATCATCGTCATCACTGTGTATTTTGCTCTGAACGTGGCCGTGGGCATATG
GTCCTCTTGTCGGGCCAGTAGGAACACGGTGAATGGCTACTTCCTGGCAGGCCGGGACATGACGTGGTGGCC
GATTGGAGCCTCCCTCTTCGCCAGCAGCGAGGGCTCTGGCCTCTTCATTGCACTGGCGGGCTCAGGCGCGGC
AGGAGGTCTGGCCGTGGCAGGCTTCGAGTGGAATGCCACGTACGTGCTGCTGGCACTGGCATGGGTGTTCGT
GCCCATCTACATCTCCTCAGAGATCGTCACCTTACCTGAGTACATTCAGAAGCGCTACGGGGGCCAGCGGAT
CCGCATGTACCTGTCTGTCCTGTCCCTGCTACTGTCTGTCTTCACCAAGATATCGCTGGACCTGTACGCGGG
GGCTCTGTTTGTGCACATCTGCCTGGGCTGGAACTTCTACCTCTCCACCATCCTCACGCTCGGCATCACAGC
CCTGTACACCATCGCAGGGGGCCTGGCTGCTGTAATCTACACGGACGCCCTGCAGACGCTCATCATGGTGGT
GGGGGCTGTCATCCTGACAATCAAAGCTTTTGACCAGATCGGTGGTTACGGGCAGCTGGAGGCAGCCTACGC
CCAGGCCATTCCCTCCAGGACCATTGCCAACACCACCTGCCACCTGCCACGTACAGACGCCATGCACATGTT
TCGAGACCCCCACACAGGGGACCTGCCGTGGACCGGGATGACCTTTGGCCTGACCATCATGGCCACCTGGTA
CTGGTGCACCGACCAGGTCATCGTGCAGCGATCACTGTCAGCCCGGGACCTGAACCATGCCAAGGCGGGCTC
CATCCTGGCCAGCTACCTCAAGATGCTCCCCATGGGCCTGATCATCATGCCGGGCATGATCAGCCGCGCATT
GTTCCCAGGTGCTCATGTCTATGAGGAGAGACACCAAGTGTCCGTCTCTCGAACAGATGATGTGGGCTGCGT
GGTGCCGTCCGAGTGCCTGCGGGCCTGCGGGGCCGAGGTCGGCTGCTCCAACATCGCCTACCCCAAGCTGGT
CATGGAACTGATGCCCATCGGTCTGCGGGGGCTGATGATCGCAGTGATGCTGGCGGCGCTCATGTCGTCGCT
GACCTCCATCTTCAACAGCAGCAGCACCCTCTTCACTATGGACATCTGGAGGCGGCTGCGTCCCCGCTCCGG
CGAGCGGGAGCTCCTGCTGGTGGGACGGCTGGTCATAGTGGCACTCATCGGCGTGAGTGTGGCCTGGATCCC
CGTCCTGCAGGACTCCAACAGCGGGCAACTCTTCATCTACATGCAGTCAGTGACCAGCTCCCTGGCCCCACC
AGTGACTGCAGTCTTTGTCCTGGGCGTCTTCTGGCGACGTGCCAACGAGCAGGGGGCCTTCTGGGGCCTGAT
AGCAGGGCTGGTGGTGGGGGCCACGAGGCTGGTCCTGGAATTCCTGAACCCAGCCCCACCGTGCCGAGAGCC
AGACACGCGGCCAGCCGTCCTGGGGAGCATCCACTACCTGCACTTCGCTGTCGCCCTCTTTGCACTCAGTGG
TGCTGTTGTGGTGGCTGGAAGCCTGCTGACCCCACCCCCACAGAGTGTCCAGATTGAGAACCTTACCTGGTG
GACCCTGGCTCAGGATGTGCCCTTGGGAACTAAAGCAGGTGATGGCCAAACACTCCAGAAACACGCCTTCTG
GGCCCGTGTCTGTGGCTTCAATGCCATCCTCCTCATGTGTGTCAACATATTCTTTTATGCCTACTTCGCCTG
ACACTGCCATCCTGGACAGAAAGGCAGGAGCTCTGAGTCC

In a search of public sequence databases, the NOV27b nucleic acid sequence, located on chromosome 17, has 903 of 1017 bases (88%) identical to a gb:GENBANK-ID:OCU08813|acc:U08813.1 mRNA from Oryctolagus cuniculus (Oryctolagus cuniculus Na+/glucose cotransporter-related protein mRNA, complete cds) (E=4.4e⁻¹⁷⁶).

The disclosed NOV27b polypeptide (SEQ ID NO:116) encoded by SEQ ID NO:115 has 612 amino acid residues and is presented in Table 27D using the one-letter amino acid code. Signal P, Psort and/or Hydropathy results predict that NOV27b has a signal peptide and is likely to be localized to the plasma membrane with a certainty of 0.8200. Alternatively, NOV27b may also localize to the endoplasmic reticulum (membrane) with a certainty of 0.6850, to the Golgi body with a certainty of 0.4600, or to the endoplasmic reticulum (lumen) with a certainty of 0.1000. The most likely cleavage site for NOV27B is between positions 42 and 43: CRA-SR.

TABLE 27D
Encoded NOV27b protein sequence.

(SEQ ID NO:116)

MAANSTSDLHTPGTQLSVADIIVITVYFALNVAVGIWSSCRASRNTVNGYFLAGRDMTWWPIGASLFASSEG
SGLFIGLAGSGAAGGLAVAGFEWNATYVLLALAWVFVPIYISSEIVTLPEYIQKRYGGQRIRMYLSVLSLLL
SVFTKISLDLYAGALFVHICLGWNFYLSTILTLGITALYTIAGGLAAVIYTDALQTLIMVVGAVILTIKAFD
QIGGYGQLEAAYAQAIPSRTIANTTCHLPRTDAMHMFRDPHTGDLPWTGMTFGLTIMATWYWCTDQVIVQRS
LSARDLNHAKAGSILASYLKMLPMGLIIMPGMISRALFPGAHVYEERHQVSVSRTDDVGCVVPSECLRACGA
EVGCSNIAYPKLVMELMPIGLRGLMIAVMLAALMSSLTSIFNSSSTLFTMDIWRRLRPRSGERELLLVGRLV
IVALIGVSVAWIPVLQDSNSGQLFIYMQSVTSSLAPPVTAVFVLGVFWRRANEQGAFWGLIAGLVVGATRLV
LEFLNPAPPCGEPDTRPAVLGSIHYLHFAVALFALSGAVVVAGSLLTPPPQSVQIENLTWWTLAQDVPLGTK
AGDGQTLQKHAFWARVCGFNAILLMCVNIFFYAYFA

A search of sequence databases reveals that the NOV27b amino acid sequence has 530 of 612 amino acid residues (86%) identical to, and 558 of 612 amino acid residues (91%) similar to, the 597 amino acid residue ptnr:SPTREMBL-ACC:Q28610 protein from Oryctolagus cuniculus (Rabbit) (Na+/Glucose Cotransporter-Related Protein) (E=1.9e⁻²⁸⁴).

NOV27b is predicted to be expressed in at least heart and kidney. The sequence is predicted to be expressed in the following tissues because of the expression pattern of (GENBANK-ID: gb:GENBANK-ID:OCU08813|acc:U08813.1) a closely related Oryctolagus cuniculus Na+/glucose cotransporter-related protein mRNA, complete cds homolog.

NOV27c

A disclosed NOV27c nucleic acid of 1741 nucleotides (also referred to as 191828203) encoding a novel sodium-glucose cotransporter-like protein is shown in Table 27E. An open reading frame was identified beginning with a ATG initiation codon at nucleotides 5-7 and ending with a TGA codon at nucleotides 1688-1690. The start and stop codons are shown in bold in Table 27E, and the 5′ and 3′ untranslated regions, if any, are underlined.

TABLE 27E
NOV27c nucleotide sequence.

(SEQ ID NO:117)

AGCCATGGCCGCCAACTCCACCAGCGACCTCCACACTCCCGGGACGCAGCTGAGCGTGGCTGACATCATCGT
CATCACTGTGTATTTTGCTCTGAATGTGGCCGTGGGCATATGGTCCTCTTGTCGGGCCAGTAGGAACACGGT
GAATGGCTACTTCCTGGCAGGCCGGGACATGACGTGGTGGCCGATTGGAGCCTCCCTCTTCGCCAGCAGCGA
GGGCTCTGGCCTCTTCATTGGACTGGCGGGCTCAGGCGCGGCAGGAGGTCTGGCCGTGGCAGGCTTCGAGTG
GAATGCCACGTACGTGCTGCTGGCACTGGCATGGGTGTTCGTGCCCATCTACATCTCCTCAGAGCTGGACCT
GTACGCGGGGGCTCTGTTTGTGCACATCTGCCTGGGCTGGAACTTCTACCTCTCCACCATCCTCACGCTCGG
CATCACAGCCCTGTACACCATCGCAGGGGGCCTGGCTGCTGTAATCTACACGGACGCCCTGCAGACGCTCAT
CATGGTGGTGGGGGCTGTCATCCTGACAATCAAAGCTTTTGACCAGATCGGTGGTTACGGGCAGCTGGAGGC
AGCCTACGCCCAGGCCATTCCCTCCAGGACCATTGCCAACACCACCTGCCACCTGCCACGTACAGACGCCAT
GCACATGTTTCGAGACCCCCACACAGGGGACCTGCCGTGGACCGGGATGACCTTTGGCCTGACCATCATGGC
CACCTGGTACTGGTGCACCGACCAGGTCATCGTGCAGCGATCACTGTCAGCCCGGGACCTGAACCATGCCAA
GGCGGGCTCCATCCTGGCCAGCTACCTCAAGATGCTCCCCATGGGCCTGATCATCATGCCGGGCATGATCAG
CCGCGCATTGTTCCCAGATGATGTGGGCTGCGTGGTGCCGTCCGAGTGCCTGCGGGCCTGCGGGGCCGAGGT
CGGCTGCTCCAACATCGCCTACCCCAAGCTGGTCATGGAACTGATGCCCATCGGTCTGCGGGGGCTGATGAT
CACAGTGATGCTGGCGGCGCTCATGTCGTCGCTGACCTCCATCTTCAACAGCAGCAGCACCCTCTTCACTAT
GGACATCTGGAGGCGGCTGCGTCCCCGCTCCGGCGAGCGGGAGCTCCTGCTGGTGGGACGGCTGGTCATAGT
GGCACTCATCGGCGTGAGTGTGGCCTGGATCCCCGTCCTGCAGGGCTCCAACAGCGGGCAACTCTTCATCTA
CATGCAGTCAGTGACCAGCTCCCTGGCCCCACCAGTGACTGCAGTCTTTGTCCTGGGCGTCTTCCGGCGACG
TGCCAACGAGCAGGGGGCCTTCTGGGGCCTGATAGCAGGGCTGGTGGTGGGGGCCACGAGGCTGGTCCTGGA
ATTCCTGAACCCAGCCCCACCGTGCGGAGAGCCAGACACGCGGCCAGCCGTCCTGGGGAGCATCCACTACCT
GCACTTCGCTGTCGCCCTCTTTGCACTCAGTGGTGCTGTTGTGGTGGCTGGAAGCCTGCTGACCCCACCCCC
ACAGAGTGTCCAGATTGACAACCTTACCTGGTGGACCCTGGCTCAGGATGTGCCCTTGGGAACTAAAGCAGG
TGATGGCCAAACACCCCAGAAACACGCCTTCTGGGCCCGCGTCTGTGGCTTCAATGCCATCCTCCTCATGTG
TGTCAACATATTCTTTTATGCCTACTTCGCCTGACACTGCCATCCTGGACACAAAGGCAGGAGCTCTGAGTT
GGCGGCCATGGCT

In a search of public sequence databases, the NOV27c nucleic acid sequence, located on chromosome 17, has 1409 of 1445 bases (97%) identical to a gb:GENBANK-ID:AX191622|acc:AX191622.1 mRNA from Homo sapiens (Sequence 144 from Patent WO0149728) (E=0.0).

A disclosed NOV27c polypeptide (SEQ ID NO:118) encoded by SEQ ID NO:117 has 561 amino acid residues and is presented in Table 27F using the one-letter amino acid code. Signal P, Psort and/or Hydropathy results predict that NOV27c has a signal peptide and is likely to be localized to the plasma membrane with a certainty of 0.8200. Alternatively, NOV27c may also localize to the endoplasmic reticulum (membrane) with a certainty of 0.6850, to the Golgi body with a certainty of 0.4600, or to the endoplasmic reticulum (lumen) with a certainty of 0.1000. The most likely cleavage site for NOV27C is between positions 42 and 43: CRA-SR.

TABLE 27F
Encoded NOV27c protein sequence.

(SEQ ID NO:118)

MAANSTSDLHTPGTQLSVADIIVITVYFALNVAVGIWSSCRASRNTVNGYFLAGRDMTWWPIGASLFASSEG
SGLFIGLAGSGAAGGLAVAGFEWNATYVLLALAWVFVPIYISSELDLYAGALFVHICLGWNFYLSTILTLGI
TALYTIAGGLAAVIYTDALQTLIMVVGAVILTIKAFDQIGGYGQLEAAYAQAIPSRTIANTTCHLPRTDAMH
MFRDPHTGDLPWTGMTFGLTIMATWYWCTDQVIVQRSLSARDLNHAKAGSILASYLKMLPMGLIIMPGMISR
ALFPDDVGCVVPSECLRACGAEVGCSNIAYPKLVMELMPIGLRGLMITVMLAALMSSLTSIFNSSSTLFTMD
IWRRLRPRSGERELLLVGRLVIVALIGVSVAWIPVLQGSNSGQLFIYMQSVTSSLAPPVTAVFVLGVFRRRA
NEQGAFWGLIAGLVVGATRLVLEFLNPAPPCGEPDTRPAVLGSIHYLHFAVALFALSGAVVVAGSLLTPPPQ
SVQIENLTWWTLAQDVPLGTKAGDGQTPQKHAFWARVCGFNAILLMCVNIFFYAYFA

A search of sequence databases reveals that the NOV27c amino acid sequence has 394 of 460 amino acid residues (85%) identical to, and 423 of 460 amino acid residues (91%) similar to, the 597 amino acid residue ptnr:SPTREMBL-ACC:Q28610 protein from Oryctolagus cuniculus (Rabbit) (Na+/Glucose Cotransporter-Related Protein) (E=2.6e⁻¹²⁵).

NOV27c is predicted to be expressed in at least heart, kidney, and colon. Expression information was derived from the tissue sources of the sequences that were included in the derivation of the sequence of CuraGen Acc. No. 191828203. The sequence is predicted to be expressed in kidney because of the expression pattern of (GENBANK-ID: gb:GENBANK-ID:OCU08813|acc:U08813.1) a closely related Oryctolagus cuniculus Na+/glucose cotransporter-related protein mRNA, complete cds homolog.

The disclosed NOV27a polypeptide has homology to the amino acid sequences shown in the BLASTP data listed in Table 27G.

TABLE 27G
BLAST results for NOV27a

| Gene Index/ | | Length | Identity | Positives | |
|---|---|---|---|---|---|
| Identifier | Protein/Organism | (aa) | (%) | (%) | Expect |
| gi\|520469\|gb\|AAA66065.1\| | 597 aa protein | 597 | 531/596 | 559/596 | 0.0 |
| (U08813) | related to | | (89%) | (93%) | |
| | Na/glucose | | | | |
| | cotransporters | | | | |
| | [Oryctolagus | | | | |
| | cuniculus] | | | | |
| gi\|16553933\|dbj\|BAB71619.1\| | unnamed protein | 517 | 440/456 | 440/456 | 0.0 |
| (AK057946) | product [Homo | | (96%) | (96%) | |
| | sapiens] | | | | |
| gi\|18203958\|gb\|AAH21357.1\| | Unknown (protein | 678 | 346/545 | 435/545 | 0.0 |
| AAH21357 | for MGC: 29197) | | (63%) | (79%) | |
| (BC021357) | [Mus musculus] | | | | |
| gi\|9588428\|emb\|CAC00574.1\| | dJ1024N4.1 (novel | 552 | 344/522 | 425/522 | e−180 |
| (AL109659) | Sodium: solute | | (65%) | (80%) | |
| | symporter family | | | | |
| | member similar to | | | | |
| | SLC5A1 (SGLT1)) | | | | |
| | [Homo sapiens] | | | | |
| gi\|2564063\|dbj\|BAA22950.1\| | Na+-glucose | 673 | 315/539 | 415/539 | e−174 |
| (AB008225) | cotransporter | | (58%) | (76%) | |
| | type 1 (SGLT-1)- | | | | |
| | like protein | | | | |
| | [Xenopus laevis] | | | | |

The homology between these and other sequences is shown graphically in the ClustalW analysis shown in Table 27H. In the ClustalW alignment of the NOV27 protein, as well as all other ClustalW analyses herein, the black outlined amino acid residues indicate regions of conserved sequence (i.e., regions that may be required to preserve structural or functional properties), whereas non-highlighted amino acid residues are less conserved and can potentially be altered to a much broader extent without altering protein structure or function.

[image]

[image]

[image]

Table 27I lists the domain description from DOMAIN analysis results against NOV27. This indicates that the NOV27 sequence has properties similar to those of other proteins known to contain this domain.

TABLE 72I
Domain Analysis of NOV27hz,1/47

gnl|Pfam|pfam00474, SSF, Sodium:solute symporter family. (SEQ ID NO:820)
CD-Length = 406 residues, 100.0% aligned
Score = 310 bits (793), Expect = 2e−85

| | | | |
|---|---|---|---|
| NOV27: | 50 | YFLAGRDMTWWPIGASLFASSEGSGLFIGLAGSGAAGGLAVAGFEWNATYVLLALAWVFV | 109 |
| | | \|\|\|\|\|\| \|\| + \| \|\| \|\| + \|+\|\|\|\|+\|\|\| \|\|\| + \| + \| \|+\| | |
| Sbjct: | | YFLAGRSMTGFVNGLSLAASYMSAASFVGLAGAGAASGLAGGLYAIGALVGVWLLLWLFA | 60 |
| NOV27: | 110 | PIYISSEIVTLPEYIQKRYGGQRIRMYLSVLSLLLSVFTKISLDLYAGALFVHICLGWNF | 169 |
| | | \| + \|+\|+\|++\|\|+\|\|+\|\| +\|\|\| \|\|\|\|\| \|\| +\|+ + \|\| + + \|\| \|+ | |
| Sbjct: | 61 | PRLRNLGAYTMPDYLRKRFGGKRILVYLSALSLLLYFFTYMSVQIVGGARLIELALGLNY | 120 |
| NOV27: | 170 | YLSTILTLGITALYTIAGGLAAVIYTDALQTLIMVVGAVILTIKAFDQIGGYGQLEAAYA | 229 |
| | | \| + +\| +\|\|+\|\| \|\| \|\| +\|\| +\| ++\|+ \| +\|\| \| \| ++\|\|\| | |
| Sbjct: | 121 | YTAVLLLGALTAIYTFFGGFLAVSWTDTIQAVLMLFGTIILMIIVFHEVGGYSSAVEKYM | 180 |
| NOV27: | 230 | QAIPSRTIANTTCHLPRTDAMHMFRDPHTGDLPWTGMTFGLTIMATWYWCTDQVIVQRSL | 289 |
| | | \| \|+ \| \| +\|+ \|\|\| \|\| \| \|+ \| \| + \|+\|\| \| | |
| Sbjct: | 181 | TADPNGVDLYT------PDGLHILRDPLTGLSLWPGLVLGTTGL--------PHILQRCL | 226 |
| NOV27: | 290 | SARDLNHAKAGSILASYLKMLPMGLIIMPGMISRALFPDDVGCVVPSECLRACGAEVGCS | 349 |
| | | +\|+\| \| \| \| + \|\| +\|+\|\|\|\|\|\|\| \|\| + \| \|\|\|\| \|\|\|\| | |
| Sbjct: | 227 | AAKD-----AKCIRCGVLILTPMFIIVMPGMISRGLFAIALAGANP----RACGTVVGCS | 277 |
| NOV27: | 350 | NIAYPKLVMELMPIGLRGLMIAVMLAALMSSLTSIFNSSSTLFTMDIWRRLRPRSGEREL | 409 |
| | | \|\|\|\|\| \| ++\| \| \|\| \|+\|+\|\|\|\|\|\|+\|\|+\|\|\| \|\|\|+ \|\| \|+++ +\| ++ \| | |
| Sbjct: | 278 | NIAYPTLAVKLGPPGLAGIMLAVMLAAIMSTLTSQLLSSSSAFTHDLYKNIRRKASATEK | 337 |
| NOV27: | 410 | LLVGRLVIVALIGVSVAWIPVLQDSNSGQLFIYMQSVTSSLAPPVTAVFVLGVFWRRANE | 469 |
| | | \|\|\|\| \|+ \|+ +\|+\| + +\| + +\| + \| \| +\| +\|\|+\| \|\| | |
| Sbjct: | 338 | ELVGRSRIIVLVVISLAILLAVQ-PAQMGIAFLVQLAFAGLGSAFLPVILLAIFWKRVNE | 396 |
| NOV27: | 470 | QGAFWGLIAG 479 | |
| | | \|\|\| \|\|+\| \| | |
| Sbjct: | 397 | QGALWGMIIG 406 | |

The gene of invention codes for a human ortholog of a rabbit sodium-glucose cotransporter (SGLT) and belongs to the large family of SGLTs that has been described to date. The rabbit gene is expressed in the kidney (Pajor,Biochim Biophys Acta Sep. 14, 1994;1194(2):349-51), and the novel gene described herein is expressed in the heart in addition to the kidney. It shows the characteristic sodium-solute symporter protein motif shared by members of the SGLT family.

SGLTs are critical in the maintenance of glucose homeostasis in the body, in a variety of tissues. Inhibitors of SGLTs are being studied in the treatment of diabetes. Treatment of Zucker diabetic fatty rats with the SGLT inhibitor T-1095 lowers both fed and fasted blood glucose levels to near-normal levels (Nawano et al., Am J Physiol Endocrinol Metab March 2000;278(3):E535-43). In streptozotocin-induced diabetic rats, T-1095 also exerts an antihyperglycemic effect which is nullified by nephrectomy, indicating that the drug acts through inhibition of renal SGLTs rather than intestinal ones (Oku et al., Biol Pharm Bull December 2000;23(12):1434-7) In addition, SGLT-1 seems to have a role in mammalian renal tubulogenesis (Yang et al., Am J Physiol Renal Physiol October 2000;279(4):F765-77).

The disclosed NOV27 nucleic acid of the invention encoding a Sodium-Glucose Cotransporter-like protein includes the nucleic acid whose sequence is provided in Table 27A, 27C, 27E or a fragment thereof. The invention also includes a mutant or variant nucleic acid any of whose bases may be changed from the corresponding base shown in Table 27A, 27C, or 27E while still encoding a protein that maintains its Sodium-Glucose Cotransporter-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 14 percent of the bases may be so changed.

The disclosed NOV27 protein of the invention includes the Sodium-Glucose Cotransporter-like protein whose sequence is provided in Table 27B, 30D, or 30F. The invention also includes a mutant or variant protein any of whose residues may be changed from the corresponding residue shown in Table 27B, 27D, or 27F while still encoding a protein that maintains its Sodium-Glucose Cotransporter-like activities and physiological functions, or a functional fragment thereof. In the mutant or variant protein, up to about 42 percent of the residues may be so changed.

The invention further encompasses antibodies and antibody fragments, such as F_{ab} or (F_{ab})₂, that bind immunospecifically to any of the proteins of the invention.

The above disclosed information suggests that this Sodium-Glucose Cotransporter-like protein (NOV27) is a member of a “Sodium-Glucose Cotransporter family”. Therefore, the NOV27 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV27 nucleic acids and proteins of the invention are useful in potential therapeutic applications implicated in diabetes, obesity, hypertension, cardiomyopathy, atherosclerosis, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, transplantation, autoimmune disease, renal artery stenosis, interstitial nephritis, glomerulonephritis, polycystic kidney disease, systemic lupus erythematosus, renal tubular acidosis, IgA nephropathy, hypercalceimia, Lesch-Nyhan syndrome, cancer, tissue degeneration, diabetic nephropathy, microvascular and macrovascular disease, and/or other diseases and pathologies.

NOV27 nucleic acids and polypeptides are further useful in the generation of antibodies that bind immuno-specifically to the novel NOV27 substances for use in therapeutic or diagnostic methods. These antibodies may be generated according to methods known in the art, using prediction from hydrophobicity charts, as described in the “Anti-NOVX Antibodies” section below. The disclosed NOV27 protein has multiple hydrophilic regions, each of which can be used as an immunogen. These novel proteins can be used in assay systems for functional analysis of various human disorders, which will help in understanding of pathology of the disease and development of new drug targets for various disorders.

NOV28

A disclosed NOV28 nucleic acid of 1560 nucleotides (also referred to as CG56185-01) encoding a MYD-1-like protein is shown in Table 28A. An open reading frame was identified beginning with a ATG initiation codon at nucleotides 31-33 and ending with a TGA codon at nucleotides 1537-1539. The start and stop codons are shown in bold in Table 28A, and the 5′ and 3′ untranslated regions, if any, are underlined.

TABLE 28A
NOV28 nucleotide sequence.

(SEQ ID NO:119)

CAGCCCTCGCGGGCGGCGTAGCCGCGGCCCATGGAGCCCGCGGGCCGGTCCCCGGCCGCCTCGGGCCGCTGC
TCTGCCTGCTGCTCCCCGCGTCCTGCGCCTGGTGGAGTGGCGGGTGAGGAGGAGCTGCAGGTGATTCAGCCT
AGAAGTCTGTATCAGTTGCAGCTGGAGAGTCGGCCGCTCTGCAGTGCACTGTGACCTCCCTGAACCCTGTG
GGGCCCATCCAACGGTTCAGAGGAGCTGGACCAGGCCGGAAATTAATCTACCATCAAAAAGAAGGCCACTTC
CCCCGGGTAACAACTGTTTCAGATCTCACAAAGAGAACCAACATGGACTTTTCCATCTGCATCAGTAACATC
ACCCCAGCAGATGCCGGCACCTACTACTGTGTGAAGTTCCAGAAAGGGAGCCCTGACGTGGAGTTGAAGTCT
GGAGCAGGCACTGAGCTGTCTGTGCGTGCCAAACCCTCTGCCCCCGTGGTATCGGGCCCCGCAGCGAGGGCC
ACACCTGACCACACAGTGAGCTTCACCTGCGAGTCTCATGGCTTCTCACCCAGAGACATCAGCCTGAAATGG
TTCAAAAATGGGAATCAGCTCTCAGACTTCCAGACCAACGTGGACCCCGCAAGAGAGAGCGTGTCCTACAGC
ATCCACAGCACAGCCAATGTGGTGCTGACCCGCGGGGACATTCACTCTCAAGTCATCTCCGAGGTGGCCCAC
GTCACCTTGCGGGGGGACTCTTTTCGTGGGACTGCCAACTTGTCTGAGACTATCCAAGTTCCACCCACCTTG
GAGGTTACTCAACAGCCCATGAGGGCAGAGAACCAGGTGAATATCACCTGCCAGGTGACGAAATTCTACCCC
CAGAGACTACAGTTGACCTGGTTGGAGAACGGCAATGTGTCCCGGACAGAAACGGCCTCAACTCTTACAGAG
AACAAGGATGGCACCTACAACTGGATGAGCTGGCTCCTGGTGAATGTATCTGCCCACAGGGATGATGTGAAG
CTCACCTGCCAGGTGGAGCATGACGGGCAGTCAGCGGTCAGCAAAAGCCATGACCTGAAGGTCTCAGCCCAC
CTGAAGGAGCAGAGCTCAAATACCGCCGCTGAGAACACTGGACCTAATGAACAGAACATCTATATTGTGGTG
GGCGTGGTGTGCACCTTGCTGGTGGCCCTACTGATGGAGGCTCTCTACCTCGTCCGAATCAGACAGAAGAAA
GCCCAGGGCTCCACTTCTTCTACAAGGTTGCATGAACCCGAGAAGAATGCCAGAAAAATAACCCAGGACACA
AATGATATCACATATGCGGACCTGAACCTGCCCAAGGGGAAGAAGCCTGCTCCCCGGGCCGCGGAGCCCAAC
AACCACACAGAGTATGCCAGCATTCAGACCAGCCTGCAGCCTGCGTCGGAGGACACCCTCACCTATGCTGAC
CTGGACATGGTGCACCTCAACCGGACCCCCAAGCAGCTGGCCCCCAAGCCCGAGCTGTCCTTCTCAGAGTAT
GCCAGCATCCAGGTCCCCAGGAAGTGAATGGGACCGTGGTTTGCTCTA

In a search of public sequence databases, the NOV28 nucleic acid sequence, located on chromosome 22, has 1466 of 1544 bases (94%) identical to a gb:GENBANK-ID:HSSIRPALP|acc:Y10375.1 mRNA from Homo sapiens (H.sapiens mRNA for SIRP-alpha1) (E=7.4e⁻³¹⁰).

The disclosed NOV28 polypeptide (SEQ ID NO:120) encoded by SEQ ID NO:119 has 503 amino acid residues and is presented in Table 28B using the one-letter amino acid code. Signal P, Psort and/or Hydropathy results predict that NOV28 has a signal peptide and is likely to be localized to the plasma membrane with a certainty of 0.4600. Alternatively, NOV28 may also localize to the endoplasmic reticulum (membrane) with a certainty of 0.1000, to the endoplasmic reticulum (lumen) with a certainty of 0.1000, or extracellularly with a certainty of 0.1000. The most likely cleavage site for NOV28 is between positions 30 and 31: VAG-EE.

TABLE 28B
Encoded NOV28 protein sequence.

(SEQ ID NO:120)

MEPAGPVPGRLGPLLCLLLPASCAWSGVAGEEELQVIQPEKSVSVAAGESAALQCTVTSLNPVGPIQRFRGA
GPGRKLIYHQKEGHFPRVTTVSDLTKRTNMDFSICISNITPADAGTYYCVKFQKGSPDVELKSGAGTELSVR
AKPSAPVVSGPAARATPDHTVSFTCESHGFSPRDISLKWFKNGNQLSDFQTNVDPARESVSYSIHSTANVVL
TRGDIHSQVICEVAHVTLRGDSFRGTANLSETIQVPPTLEVTQQPMRAENQVNITCQVTKFYPQRLQLTWLE
NGNVSRTETASTLTENKDGTYNWMSWLLVNVSAHRDDVKLTCQVEHDGQSAVSKSHDLKVSAHLKEQSSNTA
AENTGPNEQNIYIVVGVVCTLLVALLMEALYLVRIRQKKAQGSTSSTRLHEPEKNARKITQDTNDITYADLN
LPKGKKPAPRAAEPNNHTEYASIQTSLQPASEDTLTYADLDMVHLNRTPKQLAPKPELSFSEYASIQVPRK

A search of sequence databases reveals that the NOV28 amino acid sequence has458 of 503 amino acid residues (91%) identical to, and 475 of 503 amino acid residues (94%) similar to, the 503 amino acid residue ptnr:SPTREMBL-ACC:P78324 protein from Homo sapiens (Human) (Protein Tyrosine Phosphatase, Non-Receptor Type Substrate 1 Precursor (Shp Substrate-1) (Inhibitory Receptor Shps-1) (Shps-1) (Signal-Regulatory Protein Alpha-1) (SIRP-Alpha1) (MYD-1 Antigen)) (E=5.7e⁻²⁴⁷).

NOV28 is predicted to be expressed in at least myeloid, macrophages, Adrenal Gland/Suprarenal gland, Bone Marrow, Brain, Whole Organism. This information was derived by determining the tissue sources of the sequences that were included in the invention including but not limited to SeqCalling sources, Public EST sources, Literature sources, and/or RACE sources.

In addition, the sequence is predicted to be expressed in myeloid and macrophages because of the expression pattern of (GENBANK-ID: gb:GENBANK-ID:HSSIRPALP|acc: Y10375.1) a closely related H.sapiens mRNA for SIRP-alpha1 homolog.

The disclosed NOV28 polypeptide has homology to the amino acid sequences shown in the BLASTP data listed in Table 28C.

TABLE 28C
BLAST results for NOV28

| Gene Index/ | | Length | Identity | Positives | |
|---|---|---|---|---|---|
| Identifier | Protein/Organism | (aa) | (%) | (%) | Expect |
| gi\|14771369\|ref\|XP_{—} | hypothetical | 504 | 458/504 | 476/504 | 0.0 |
| 044897.1\| | protein XP_044897 | | (90%) | (93%) | |
| (XM_044897) | [Homo sapiens] | | | | |
| gi\|4758978\|ref\|NP_004639.1\| | protein tyrosine | 503 | 458/503 | 475/503 | 0.0 |
| (NM_004648) | phosphatase, non- | | (91%) | (94%) | |
| | receptor type | | | | |
| | substrate 1; SHP | | | | |
| | substrate-1 [Homo | | | | |
| | sapiens] | | | | |
| gi\|6624134\|gb\|AAF19260.1\| | similar to SHPS-1 | 402 | 402/402 | 402/402 | 0.0 |
| AC004832_5 | [Homo sapiens]; | | (100%) | (100%) | |
| (AC004832) | similar to | | | | |
| | BAA12974.1 | | | | |
| | (PID: g1864011) | | | | |
| gi\|2842392\|emb\|CAA71944.1\| | MyD-1 antigen | 429 | 391/429 | 407/429 | 0.0 |
| (Y11047) | [Homo sapiens] | | (91%) | (94%) | |
| gi\|2842390\|emb\|CAA71942.1\| | MyD-1 antigen | 506 | 373/510 | 415/510 | 0.0 |
| (Y11045) | [Bos taurus] | | (73%) | (81%) | |

The homology between these and other sequences is shown graphically in the ClustalW analysis shown in Table 28D. In the ClustalW alignment of the NOV28 protein, as well as all other ClustalW analyses herein, the black outlined amino acid residues indicate regions of conserved sequence (i.e., regions that may be required to preserve structural or functional properties), whereas non-highlighted amino acid residues are less conserved and can potentially be altered to a much broader extent without altering protein structure or function.

[image]

[image]

Tables 28E-F list the domain descriptions from DOMAIN analysis results against NOV28. This indicates that the NOV28 sequence has properties similar to those of other proteins known to contain this domain.

TABLE 28E
Domain Analysis of NOV28

gnl|Smart|smart00408, IGc1, Immunoglobulin C-Type (SEQ ID NO:821)
CD-Length = 75 residues, 94.7% aligned
Score = 508 bits (120), Expect = 2e-07

| | | | |
|---|---|---|---|
| NOV28: | 267 | QVNITCQVTKFYPQRLQLTWLENGNVSRTETAST-LTENKDGTYNWMSWLLVNVSAHRDD | 325 |
| | | + \| \|\| \|\|\| + +\|\|\|+\|\| + +\| ++\|\|\|\|\| \|+\| \|+ \| | |
| Sbjct: | 1 | PATLVCLVTGFYPPDITVTWLKNGQEVTSGVKTTDPLKDKDGTYFLSSYLTVSASTWESG | 60 |
| NOV28: | 326 | VKLTCQVEHDG 336 | |
| | | \|\|\|\| \|+\| | |
| Sbjct: | 61 | DVYTCQVTHEG 71 | |

TABLE 28F
Domain Analysis of NOV28

gnl|Smart|smart00407, IGc1, Immunoglobulin C-Type (SEQ ID NO:821)
CD-Length = 75 residues, 96.0% aligned
Score = 47.8 bits (112), Expect = 2e−06

| | | | |
|---|---|---|---|
| NOV28: | 164 | TVSFTCESHGFSPRDISLKWFKNGNQLSDFQTNVDPARES-VSYSIHSTANVVLTRGDIH | 222 |
| | | + \| \|\| \| \|\|++ \| \|\|\| +++ \|\| ++ +\| + \| \| + + | |
| Sbjct: | 1 | PATLVCLVTGFYPPDITVTWLKNGQEVTSGVKTTDPLKDKDGTYFLSSYLTVSASTWESG | 60 |
| NOV28: | 223 | SQVICEVAHVTL 234 | |
| | | \|+\| \| \| | |
| Sbjct: | 61 | DVYTCQVTHEGL 72 | |

Protein tyrosine phosphatases (PTPases), such as SHP-1 and SHP-2; that contain Src homology 2 (SH2) domains play important roles in growth factor and cytokine signal transduction pathways. A protein of approximately 115 to 120 kDa that interacts with SHP-1 and SHP-2 was purified from v-src-transformed rat fibroblasts (SR-3Y1 cells), and the corresponding cDNA was cloned. The predicted amino acid sequence of the encoded protein, termed SHPS-1 (SHP substrate 1), suggests that it is a glycosylated receptor-like protein with three immunoglobulin-like domains in its extracellular region and four YXX(L/V/I) motifs, potential tyrosine phosphorylation and SH2-domain binding sites, in its cytoplasmic region. Various mitogens, including serum, insulin, and lysophosphatidic acid, or cell adhesion induced tyrosine phosphorylation of SHPS-1 and its subsequent association with SHP-2 in cultured cells. Thus, SHPS-1 may be a direct substrate for both tyrosine kinases, such as the insulin receptor kinase or Src, and a specific docking protein for SH2-domain-containing PTPases. In addition, we suggest that SHPS-1 may be a potential substrate for SHP-2 and may function in both growth factor- and cell adhesion-induced cell signaling. (Fujioka et al. Mol Cell Biol. December 1996;16(12):6887-99.)

The rat OX41 antigen is a cell surface protein containing three immunoglobulin superfamily domains and intracellular immunoreceptor tyrosine-based inhibitory motifs (ITIM). It is a homologue of the human signal-regulatory protein (SIRP) also known as SHPS-1, BIT or MFR. Cell activation-induced phosphorylation of the intracellular ITIM motifs induces association with the tyrosine phosphatases SHP-1 and SHP-2. To identify the physiological OX41 ligand, recombinant OX41-CD4d3+4 fusion protein was coupled to fluorescent beads to produce a multivalent cell binding reagent. The OX41-CD4d3+4 beads bound to thymocytes and concanavalin A-stimulated splenocytes. This interaction was blocked by the monoclonal antibody (mAb) OX101. Affinity chromatography with OX101 mAb and peptide sequencing revealed the rat SIRP ligand to be CD47 (integrin-associated protein). A direct interaction between human SIRP and human CD47 was demonstrated using purified recombinant proteins and surface plasmon resonance ruling out the involvement of other proteins known to be associated with CD47. The affinity of the SIRP/CD47 interaction was K(d) approximately 8 microM at 37 degrees C. with a k(off )>/=2.1 s(−1). The membrane-distal SIRP V-like domain was sufficient for binding to CD47.(Vernon-Wilson EF, et al. Eur J Immunol. August 2000;30(8):2130-7.)

The transmembrane glycoprotein SHPS-1 binds the protein tyrosine phosphatase SHP-2 and serves as its substrate. Although SHPS-1 has been implicated in growth factor- and cell adhesion-induced signaling, its biological role has remained unknown. Fibroblasts homozygous for expression of an SHPS-1 mutant lacking most of the cytoplasmic region of this protein exhibited increased formation of actin stress fibers and focal adhesions. They spread more quickly on fibronectin than did wild-type cells, but they were defective in subsequent polarized extension and migration. The extent of adhesion-induced activation of Rho, but not that of Rac, was also markedly reduced in the mutant cells. Activation of the Ras-extracellular signal-regulated kinase signaling pathway and of c-Jun N-terminal kinases by growth factors was either unaffected or enhanced in the mutant fibroblasts. These results demonstrate that SHPS-1 plays crucial roles in integrin-mediated cytoskeletal reorganization, cell motility and the regulation of Rho, and that it also negatively modulates growth factor-induced activation of mitogen-activated protein kinases. (Inagaki, A. et al., EMBO J. Dec. 15, 2000;19(24):6721-31.)

Machida K. et al. (Oncogene. Mar. 23, 2000;19(13):1710-8.) investigated the effect of cell transformation by v-src on the expression and tyrosine phosphorylation of SHPS-1, a putative docking protein for SHP-1 and SHP-2. They found that transformation by v-src virtually inhibited the SHPS-1 expression at mRNA level. While nontransforming Src kinases including c-Src, nonmyristoylated forms of v-Src had no inhibitory effect on SHPS-1 expression, transforming Src kinases including wild-type v-Src and chimeric mutant of c-Src bearing v-Src SH3 substantially suppressed the SHPS-1 expression. In cells expressing temperature sensitive mutant of v-Src, suppression of the SHPS-1 expression was temperature-dependent. In contrast, tyrosine phosphorylation of SHPS-1 was rather activated in cells expressing c-Src or nonmyristoylated forms of v-Src. SHPS-1 expression in SR3Y1 was restored by treatment with herbimycin A, a potent inhibitor of tyrosine kinase, or by the expression of dominant negative form of Ras. Contrary, active form of Mekl markedly suppressed SHPS-1 expression. Finally, overexpression of SHPS-1 in SR3Y1 led to the drastic reduction of anchorage independent growth of the cells. Taken together, their results suggest that the suppression of SHPS-1 expression is a pivotal event for cell transformation by v-src, and the Ras-MAP kinase cascade plays a critical role in the suppression.

SHPS-1 (SH2-domain bearing protein tyrosine phosphatase (SHP) substrate-l), a member of the inhibitory-receptor superfamily that is abundantly expressed in macrophages and neural tissue, appears to regulate intracellular signaling events downstream of receptor protein-tyrosine kinases and integrin-extracellular matrix molecule interactions. To investigate the function of SHPS-1 in a hematopoietic cell line, SHPS-1 was expressed in Ba/F3 cells, an IL-3-dependent pro-B-cell line that lacks endogenous SHPS-1 protein. Interestingly, expression of either SHPS-1, or a mutant lacking the intracellular domain of SHPS-1 (DeltaCT SHPS-1), resulted in the rapid formation of macroscopic Ba/F3 cell aggregates. As the integrin-associated protein/CD47 was shown to be a SHPS-1 ligand in neural cells, Babic, J. et al. (J Immunol. Apr. 1, 2000;164(7):3652-8.) investigated whether CD47 played a role in the aggregation of SHPS-1-expressing Ba/F3 cells. In support of this idea, aggregate formation was inhibited by an anti-CD47 Ab. Furthermore, erythrocytes from control, but not from CD47-deficient mice, were able to form rosettes on SHPS-1-expressing Ba/F3 cells. Because erythrocytes do not express integrins, this result suggested that SHPS-1-CD47 interactions can take place in the absence of a CD47-integrin association. They also present evidence that the amino-terminal Ig domain of SHPS-1 mediates the interaction with CD47. Although SHPS-1-CD47 binding likely triggers bidirectional intracellular signaling processes, these results demonstrate that this interaction can also mediate cell-cell adhesion.

Inhibitory immunoreceptors downregulate signaling by recruiting Src homology 2 (SH2) domain-containing tyrosine and/or lipid phosphatases to activating receptor complexes [1]. There are indications that some inhibitory receptors might also perform other functions [2] [3]. In adherent macrophages, two inhibitory receptors, SHPS-1 and PIR-B, are the major proteins binding to the tyrosine phosphatase SHP-1. SHPS-1 also associates with two tyrosine-phosphorylated proteins (pp55 and pp130) and a protein tyrosine kinase [4]. Here, Timms, J F. et al. (Curr Biol. Aug. 26, 1999;9(16):927-30.) have identified pp55 and pp130 as the adaptor molecules SKAP55hom/R (Src-kinase-associated protein of 55 kDa homologue) and FYB/SLAP-130 (Fyn-binding protein/SLP-76-associated protein of 130 kDa), respectively, and the tyrosine kinase activity as PYK2. Two distinct SHPS-1 complexes were formed, one containing SKAP55hom/R and FYB/SLAP-130, and the other containing PYK2. Recruitment of FYB/SLAP-130 to SHPS-1 required SKAP55hom/R, whereas PYK2 associated with SHPS-1 independently. Formation of both complexes was independent of SHP-1 and tyrosine phosphorylation of SHPS-1. Finally, tyrosine phosphorylation of members of the SHPS-1 complexes was regulated by integrin-mediated adhesion. Thus, SHPS-1 provides a scaffold for the assembly of multi-protein complexes that might both transmit adhesion-regulated signals and help terminate such signals through SHP-1-directed dephosphorylation. Other inhibitory immunoreceptors might have similar scaffold-like functions.

SHPS-1 (or SIRP) is a member of the immunoglobulin (Ig) superfamily abundantly expressed in neurons and other cell types. Within its cytoplasmic domain, it possesses at least two immunoreceptor tyrosine-based inhibitory motifs, which are targets for tyrosine phosphorylation and mediate the recruitment of SHP-2, an Src homology 2 (SH2) domain-containing protein-tyrosine phosphatase. Since other immunoreceptor tyrosine-based inhibitory motifs-containing receptors have critical roles in the negative regulation of hemopoietic cell functions, the expression of SHPS-1 in cells of hematological lineages was examined. By analyzing a panel of hemopoietic cell lines, evidence was provided that SHPS-1 is abundantly expressed in macrophages and, to a lesser extent, in myeloid cells. No expression was detected in T-cell or B-cell lines. Expression of SHPS-1 could also be documented in normal ex vivo peritoneal macrophages. Further studies showed that SHPS-1 was an efficient tyrosine phosphorylation substrate in macrophages. However, unlike in non-hemopoietic cells, tyrosine-phosphorylated SHPS-1 in macrophages associated primarily with SHP-1 and not SHP-2. Finally, analyses allowed identification of several isoforms of SHPS-1 in mouse cells. In part, this heterogeneity was due to differential glycosylation of SHPS-1. Additionally, it was caused by the production of at least two distinct shps-1 transcripts, coding for SHPS-1 polypeptides having different numbers of Ig-like domains in the extracellular region. Taken together, these findings indicate that SHPS-1 is likely to play a significant role in macrophages, at least partially as a consequence of its capacity to recruit SHP-1. Veilette, A. et al. (J Biol Chem. Aug. 28, 1989;273(35):22719-28.)

SHPS-1 is a 120 kDa glycosylated receptor-like protein that contains immunoglobulin-like domains in its extracellular region and four potential tyrosine phosphorylation for SH2 domain binding sites in its cytoplasmic region. Epidermal growth factor (EGF) stimulated the rapid tyrosine phosphorylation of SHPS-1 and subsequent association of SHPS-1 with SHP-2, a protein tyrosine phosphatase containing SH2 domains, in Chinese hamster ovary cells overexpressing human EGF receptors. In the cells overexpressing SHPS-1, the tyrosine phosphorylation of SHPS-1 was more evident than that observed in parent cells. However, overexpression of SHPS-1 alone did not affect the activation of MAP kinase in response to EGF. These results suggest that SHPS-1 may be involved in the recruitment of SHP-2 from the cytosol to the plasma membrane in response to EGF. Copyright 1997 Academic Press. Ochi, F. et al. (Biochem Biophys Res Commun. Oct. 20, 1997;239(2):483-7.)

The immune system recognizes invaders as foreign because they express determinants that are absent on host cells or because they lack ‘markers of self’ that are normally present. Oldenborg et al. (2000) demonstrated that CD47 functions as a marker of self on murine red blood cells. Red blood cells that lack CD47 were rapidly cleared from the bloodstream by splenic red pulp macrophages. CD47 on normal red blood cells prevented this elimination by binding to the inhibitory receptor signal regulatory protein alpha (SIRP-alpha). Thus, Oldenborg et al. (2000) concluded that macrophages may use a number of nonspecific activating receptors and rely on the presence or absence of CD47 to distinguish self from foreign. Oldenborg et al. (2000) suggested that CD47-SIRP-alpha may represent a potential pathway for the control of hemolytic anemia.

The disclosed NOV28 nucleic acid of the invention encoding a MYD-1-like protein includes the nucleic acid whose sequence is provided in Table 28A or a fragment thereof. The invention also includes a mutant or variant nucleic acid any of whose bases may be changed from the corresponding base shown in Table 28A while still encoding a protein that maintains its MYD-1-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 6 percent of the bases may be so changed.

The disclosed NOV28 protein of the invention includes the MYD-1-like protein whose sequence is provided in Table 28B. The invention also includes a mutant or variant protein any of whose residues may be changed from the corresponding residue shown in Table 28B while still encoding a protein that maintains its MYD-1-like activities and physiological functions, or a functional fragment thereof. In the mutant or variant protein, up to about 27 percent of the residues may be so changed.

The invention further encompasses antibodies and antibody fragments, such as F_{ab} or (F_{ab})₂, that bind immunospecifically to any of the proteins of the invention.

The above disclosed information suggests that this MYD-1-like protein (NOV28) is a member of a “MYD-1 family”. Therefore, the NOV28 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV28 nucleic acids and proteins of the invention are useful in potential therapeutic applications implicated in epilepsy, eating disorders, schizophrenia, ADD, and cancer; heart disease; inflammation and autoimmune disorders including Crohn's disease, IBD, allergies, rheumatoid and osteoarthritis, inflammatory skin disorders, allergies, blood disorders; psoriasis colon cancer, leukemia AIDS; thalamus disorders; metabolic disorders including diabetes and obesity; lung diseases such as asthma, hemolytic anemia, emphysema, cystic fibrosis, and cancer; pancreatic disorders including pancreatic insufficiency and cancer; and prostate disorders including prostate cancer, and/or other diseases and pathologies.

NOV28 nucleic acids and polypeptides are further useful in the generation of antibodies that bind immuno-specifically to the novel NOV28 substances for use in therapeutic or diagnostic methods. These antibodies may be generated according to methods known in the art, using prediction from hydrophobicity charts, as described in the “Anti-NOVX Antibodies” section below. The disclosed NOV28 protein has multiple hydrophilic regions, each of which can be used as an immunogen. These novel proteins can be used in assay systems for functional analysis of various human disorders, which will help in understanding of pathology of the disease and development of new drug targets for various disorders.

NOV29

NOV29 includes three novel CRAL-TRIO-like proteins disclosed below. The disclosed sequences have been named NOV29a, NOV29b, and NOV29c.

NOV29a

A disclosed NOV29a nucleic acid of 1327 nucleotides (also referred to as CG56187-01) encoding a CRAL-TRIO-like protein is shown in Table 29A. An open reading frame was identified beginning with a ATG initiation codon at nucleotides 16-18 and ending with a TGA codon at nucleotides 1261-1263. The start and stop codons are shown in bold in Table 29A, and the 5′ and 3′ untranslated regions, if any, are underlined.

TABLE 29A
NOV29a nucleotide sequence.

(SEQ ID NO:121)

GGAGTTGACTGGTGGATGATGTGGGAAGGGTTAGGGGCCGGGTTGGTGGCCCCCGAGGTCATGAGACCTCCG
CCGACCATCAGATCCTCCTCCGCTCAGTTCCGGGAGAACCTCCAGGACCTGCTGCCCATACTGCCCAATGCT
GATGACTACTTCCTCCTGCGCTGCCTGGCAGCTCGAAACTTTGACCTGCAGAAATCCGAAGACATGCTCCGA
AGGCACATGGAGTTCCGGAAGCAACAAGACCTGGACAACATTGTCACATGGCAGCCCCCTGAGGTGGTCATC
CAGCTGTATGACTCGGCTGGTCTTTGTGGCTACGACTACGAAGGCTGCCCTGTGTACTTCAACATCATTGGG
TCCCTCGACCCCAAGGGTCTCCTGCTGTCAGCCTCCAAGCAGGATATGATCCGGAAGCGCATCAAAGTCTGT
GAGCTGCTGTTGCATGAGTGTGAGCTGCAAACTCAGAAGCTGGGCAGGAAGATCGAGATGGCGCTGATGGTG
TTTGACATGGAGGGGCTGAGCCTGAAACACCTGTGGAAGCCAGCTGTGGAGGTCTACCAGCAGTTTTTTAGC
ATCCTGGAAGCAAATTATCCTGAGACCCTGAAGAATTTAATTGTTATTCGAGCCCCAAAACTGTTCCCCGTG
GCCTTCAACTTGGTCAAGTCGTTCATGAGTGAGGAGACACGCAGGAAGATTGTGATTCTGGCAGACAACTGG
AAGCAGGAGCTGACAAAATTCATCAGCCCCGACCAGCTGCCTGTGGAGTTTGGGGGGACCATGACTGACCCC
GATGGCAACCCCAAGTGCCTGACCAAGATCAACTATCGGGGTGAGGTGCCCAAGAGCTACTACCTGTGCGAG
CAGGTGAGGCTGCAGTATGAGCACACGAGGTCCGTGGGCCGCGGCTCCTCCCTGCACGTGGAGAACGAGATC
CTGTTCCCGGGCTGTGTGCTCAGGTGGCAGTTTGCTTCAGATGGTGGGGACATCGGCTTTGGGGTTTTCCTG
AAGACCAAGATGGGGGAGCAGCAGAGTGCTAGGGAGATGACGGAGGTGCTGCCCAGCCAGCGCTACAATGCC
CACATGGTGCCTGAGGATGGGAGCCTCACCTGCCTCCAGGCTGGCGTCCTGCGCTTCGACAACACCTACAGC
CGGATGCATGCCAAGAAGCTCAGCTACACTGTGGAGGTGCTGCTTCCCGACAAGGCCTCTGAGGAGACGCTG
CAGAGTCTCAAGGCGATGAGACCCTCCCCAACACAGTGAAGACCCCAGCCACCTCTACCTGTGCACTCCAAC
CCCTTCACACCCACCCCTCTGACCCCTGCCT

In a search of public sequence databases, the NOV29a nucleic acid sequence, located on chromosome 22, has 935 of 1263 bases (74%) identical to a gb:GENBANK-ID:RNO132352|acc:AJ132352.1 mRNA from Rattus norvegicus (Rattus norvegicus mRNA for 45 kDa secretory protein, partial) (E=4.0e⁻¹³²).

A disclosed NOV29a polypeptide (SEQ ID NO:122) encoded by SEQ ID NO:121 has 415 amino acid residues and is presented in Table 29B using the one-letter amino acid code. Signal P, Psort and/or Hydropathy results predict that NOV29a has no signal peptide and is likely to be localized extracellularly with a certainty of 0.6500. Alternatively, NOV29a may also localize to the mitochondrial membrane spacewith a certainty of 0.1000, to the lysosome (lumen) with a certainty of 0.1000, or to the microbody (peroxisome) with a certainty of 0.0348.

TABLE 29B
Encoded NOV29a protein sequence.

(SEQ ID NO:122)

MMWEGLGAGLVAPEVMRAPPTIRSSSAQFRENLQDLLPILPNADDYFLLRWLAARNFDLQKSEDMLRRHMEF
RKQQDLDNIVTWQPPEVVIQLYDSGGLCGYDYEGCPVYFNIIGSLDPKGLLLSASKQDMIRKRIKVCELLLH
ECELQTQKLGRKIEMALMVFDMEGLSLKHLWKPAVEVYQQFFSILEANYPETLKNLIVIRAPKLFPVAFNLV
KSFMSEETRRKIVILGDNWKQELTKFISPDQLPVEFGGTMTDPDGNPKCLTKINYGGEVPKSYYLCEQVRLQ
YEHTRSVGRGSSLQVENEILFPGCVLRWQFASDGGDIGFGVFLKTKMGEQQSAREMTEVLPSQRYNAHMVPE
DGSLTCLQAGVLRFDNTYSRNHAKKLSYTVEVLLPDKASEETLQSLKAMRPSPTQ

A search of sequence databases reveals that the NOV29a amino acid sequence has 387 of 397 amino acid residues (97%) identical to, and 390 of 397 amino acid residues (98%) similar to, the 406 amino acid residue ptnr:SPTREMBL-ACC:Q9UDX3 protein from Homo sapiens (Human) (WUGSC:H_DJ0539M06.4 PROTEIN) (E=7.2e⁻²⁰⁸).

NOV29a is predicted to be expressed in at least Bone, liver, brain, and prostate. This information was derived by determining the tissue sources of the sequences that were included in the invention including but not limited to SeqCalling sources, Public EST sources, Literature sources, and/or RACE sources.

In addition, the sequence is predicted to be expressed in Bone because of the expression pattern of (GENBANK-ID: gb:GENBANK-ID:RNO132352|acc: AJ132352.1) a closely related Rattus norvegicus mRNA for 45 kDa secretory protein, partial homolog.

NOV29b

A disclosed NOV29b nucleic acid of 1305 nucleotides (also referred to as CG56187-03) encoding a CRAL-TRIO-like protein is shown in Table 29C. An open reading frame was identified beginning with a ATG initiation codon at nucleotides 14-16 and ending with a TGA codon at nucleotides 1262-1264. The start and stop codons are shown in bold in Table 29C, and the 5′ and 3′ untranslated regions, if any, are underlined.

TABLE 29C
NOV29b nucleotide sequence

(SEQ ID NO:123)

AGTTGACTGGTGGATGATGTGGGAAGGGTTAGGGGCGGGGTTGGTGGCCCCCGAGGTCATGAGAGCTCCGCC
GACCATCAGATCCTCCTCCGCTCAGTTCCGGGAGAACCTCCAGGACCTGCTGCCCATACTGCCCAATGCTGA
TGACTACTTCCTCCTGCGCTGGCTGCGAGCTCGAAACTTTGACCTGCAGAAATCCGAAGACATGCTCCGAAG
GCACATGGAGTTCCGGAAGCAACAAGACCTGGACAACATTGTCACATGGCAGCCCCCTGAGGTCATCCAGCT
GTATGACTCGGGTGGTCTTTGTGGCTACGACTACGAAGGCTGCCCTGTGTACTTCAACATCATTGGGTCCCT
CGACCCCAAGGGTCTCCTGCTGTCAGCCTCCAAGCAGGATATGATCCGGAAGCGCATCAAAGTCTGTGAGCT
GCTCTTGCATGACTGTGAGCTGCAAACTCAGAAGCTGGGCAGGAAGATCGAGATGGCCCTGATGGTGTTTGA
CATGGAGGGGCTGAGCCTGAAACACCTGTGGAAGCCAGCTGTGGAGGTCTACCAGCAGTTTTTTAGCATCCT
GGAAGCAAATTATCCTGAGACCCTGAAGAATTTAATTGTTATTCGAGCCCCAAAACTGTTCCCCGTGGCCTT
CAACTTGGTCAAGTCGTTCATGAGTGAGGAGACACGCAGGAAGATTGTGATTCTGGGAGACAACTGGAAGCA
GGAGCTGACAAAATTCATCAGCCCCGACCAGCTGCCTGTGGAGTTTGGGGCGACCATGACTGACCCCGATGG
CCACCCCAAGTGCCTGACCAAGATCAACTATGGGGGTGAGGTGCCCAAGAGCTACTACCTGTGCGAGCAGGT
GAGGCTGCAGTATGAGCACACGAGGTCCGTGGGCCGCGGCTCCTCCCTGCAGGTGGAGAACGAGATCCTGTT
CCCGGGCTGTGTGCTCAGGTGGCAGTTTGCTTCAGATGGTGGGGACATCGGCTTTGGGGTTTTCCTGAAGAC
CAAGATGGCGGAGCAGCAGAGTGCTAGGGAGATGACGGAGGTGCTGCCCAGCCAGCGCTACAATGCCCACAT
GGTGCCTGAGGATGGGAGCCTCACCTGCCTCCAGGCTGGCGTCTATGTCCTGCGCTTCGACAACACCTACAG
CCGGATGCATGCCAAGAAGCTCAGCTACACTGTGGAGGTGCTGCTTCCCGACAAGGCCTCTGAGGAGACGCT
GCAGAGTCTCAGGCGATGAGACCCTCCCCAACACAGTGAAGACCCCAGCCACCTCCACCTGTGCACTCCAA
CCCCTTCAC

In a search of public sequence databases, the NOV29b nucleic acid sequence, located on chromosome 22, has 906 of 1212 bases (74%) identical to a gb:GENBANK-ID:BC005759|acc:BC005759.1 mRNA from Mus musculus (Mus musculus, clone MGC:6302, mRNA, complete cds) (E=2.0e⁻¹³⁷).

A disclosed NOV29b polypeptide (SEQ ID NO:124) encoded by SEQ ID NO:123 has 416 amino acid residues and is presented in Table 29D using the one-letter amino acid code. Signal P, Psort and/or Hydropathy results predict that NOV29b has no signal peptide and is likely to be localized extracellularly with a certainty of 0.4500. Alternatively, NOV29b may also localize to the mitochondrial membrane spacewith a certainty of 0.1000, to the lysosome (lumen) with a certainty of 0.1000, or to the microbody (peroxisome) with a certainty of 0.0779.

TABLE 29D
Encoded NOV29b protein sequence.

(SEQ ID NO:124)

MMWEGLGAGLVAPEVMRAPPTIRSSSAQFRENLQDLLPILPNADDYFLLRWLRARNFDLQKSEDMLRRHMEF
RKQQDLDNIVTWQPPEVIQLYDSGGLCGYDYEGCPVYFNIIGSLDPKGLLLSASKQDMIRKRIKVCELLLHE
CELQTQKLGRKIEMALMVFDMEGLSLKHLWKPAVEVYQQFFSILEANYPETLKNLIVIRAPKLFPVAFNLVK
SFMSEETRRKIVILGDNWKQELTKFISPDQLPVEFGGTMTDPDCHPRCLTKINYGGEVPKSYYLCEQVRLQY
EHTRSVGRGSSLQVENEILFPGCVLRWQFASDGGDTGFGVFLKTKMGEQQSAREMTEVLPSQRYNAHMVPED
GSLTCLQAGVYVLRFDNTYSRMHAKKLSYTVEVLLPDKASEETLQSLKAMRPSPTQ

A search of sequence databases reveals that the NOV29b amino acid sequence has 906 of 1212 amino acid residues (74%) identical to, and 906 of 1212 amino acid residues (74%) similar to, the 2529 amino acid residue gb:GENBANK-ID:BC005759|acc:BC005759.1 protein from Mus musculus (Mus musculus, clone MGC:6302, mRNA, complete cds) (E=2.0e⁻¹³⁷).

NOV29b is predicted to be expressed in at least Bone, liver, brain, and prostate. The sequence is predicted to be expressed inbone because of the expression pattern of (GENBANK-ID: gb:GENBANK-ID:BC005759|acc:BC005759.1) a closely related Mus musculus, clone MGC:6302, mRNA, complete cds homolog.

NOV29c

A disclosed NOV29c nucleic acid of 1218 nucleotides (also referred to as CG56189-01) encoding a CRAL-TRIO-like protein is shown in Table 29E. An open reading frame was identified beginning with a ATG initiation codon at nucleotides 1-3 and ending with a TAG codon at nucleotides 1216-1218. The start and stop codons are shown in bold in Table 29E, and the 5′ and 3′ untranslated regions, if any, are underlined.

TABLE 29E
NOV29c nucleotide sequence.

(SEQ ID NO:125)

ATGTTCCGGGAGAACATCCAAGATGTGCTATCTGCGCTGCCCAATCCTGATGACTACTTCCTCCTGCGCTGG
CTCCAAGCTCGGAGCTTTGACCTGCAGAAATCAGAGGACATGCTGAGGAAGCATATGGAGTTCCGGAAGCAA
CAAGACCTGGCCAACATCCTTGCCTGGCAGCCCCCAGAGGTGGTCAGGCTGTACAACGCTAACGGCATATGC
GGCCACGACGGTGAGGGCAGCCCTGTCTGCTACCACATTGTGGGAAGCCTGGACCCCAAAGGCCTCTTGCTC
TCAGCCTCCAAACAGGAGTTGCTCAGGGACAGCTTCCGGAGCTGCGAGCTGCTCCTGCGGGAGTGTGACCTG
CAGAGTCAGAAGCTGGGGAAGAGGGTGGAGAAAATCATAGCTATTTTTGGTCTCGAAGGGCTGGGCCTGAGG
GATCTGTGGAAGCCAGGAATAGAGCTTCTCCAGGAGTTTTTCTCAGCACTTGAAGCAAATTACCCTGAGATC
TTGAAGAGTTTAATTGTTGTGAGAGCCCCCAAGCTATTCGCCGTAGCCTTCAACCTGGTCAAGTCTTACATG
AGTGAAGAGACACGCAGGAAGGTGGTGATTCTCGGAGATCTGATGGTTCCTGCATCCGAAGGTGTAGGGCAC
CCAACTGGTGTTGAGGGCCCACTGCCTGGTGGGCTGCCAGACAACTGGAAGCAGGAGCTGACAAAATTCATC
AGCCCCGACCAGCTGCCCGTGGAGTTTGGGGGGACCATGACTGACCCCGATGGCAACCCCAAGTGCCTGACC
AAGATCAACTACGGGGGTGAGGTGCCCAAGAGCTACTACCTGTGCAAGCAGGTGAGGCTGCAGTATGAGCAC
ACGAGGTCCGTGGGCCGCGCCTCCTCCCTGCAGGTGGAGAACGAGATCCTGTTCCCGGCCTGTGTGCTCAGG
TGGCAGTTTGCTTCAGATGGTGGGGACATTGGCTTTGGGGTTTTCCTGAAGACCAAGATGGGGGAGCGGCAG
AGGGCTAGGGAGATGACAGAGGTGCTGCCCAGCCAGCGCTACAATGCCCACATGGTGCCTGAAGATGGGATT
CTCACCTGCCTCCAGGCCGGCAGCTATGTCCTGAGGTTTTACAACACCTACAGCCTGGTTCATTCTAAACGC
ATCAGCTACACCGTGGAGGTACTGCTCCCAGACCAAACCTTCATGGAGAAGATGGAGAAATTCTAG

In a search of public sequence databases, the NOV29c nucleic acid sequence, located on chromosome 22, has 418 of 532 bases (78%) identical to a gb:GENBANK-ID:HS130H16A|acc:AL096881.1 mRNA from Homo sapiens (Novel human mRNA similar to Rattus norvegicus 45 kDa secretory protein, AJ132352) (E=4.9e⁻¹²⁹).

The disclosed NOV29c polypeptide (SEQ ID NO:126) encoded by SEQ ID NO:125 has 405 amino acid residues and is presented in Table 29F using the one-letter amino acid code. Signal P, Psort and/or Hydropathy results predict that NOV29c has no signal peptide and is likely to be localized extracellularly with a certainty of 0.4500. Alternatively, NOV29c may also localize to the microbody (peroxisome) with a certainty of 0.2010, to the mitochondrial matrix space with a certainty of 0.1000, or to the lysosome (lumen) with a certainty of 0.1000.

TABLE 29F
Encoded NOV29c protein sequence.

(SEQ ID NO:126)

MFRENIQDVLSALPNPDDYFLLRWLQARSFDLQKSEDMLRKHMEFRKQQDLANILAWQPPEVVRLYNANGIC
GHDGEGSPVWYHIVGSLDPKGLLLSASKQELLRDSFRSCELLLRECELQSQKLGKRVEKIIAIFGLEGLGLR
DLWKPGIELLQEFFSALEANYPEILKSLIVVRAPKLFAVAFNLVKSYMSEETRRKVVILGDLMVPASEGVGH
PTGVECPLPGGLPDNWKQELTKFISPDQLPVEFGGTMTDPDGNPKCLTKINYGGEVPKSYYLCKQVRLQYEH
TRSVGRGSSLQVENEILFPGCVLRWQFASDGGDIGFGVFLKTKMGERQRAREMTEVLPSQRYNAHNVPEDGI
LTCLQAGSYVLRFYNTYSLVHSKRISYTVEVLLPDQTFMEKMEKF

A search of sequence databases reveals that the NOV29c amino acid sequence has 157 of 176 amino acid residues (89%) identical to, and 166 of 176 amino acid residues (94%) similar to, the 406 amino acid residue ptnr:SPTREMBL-ACC:Q9UDX3 protein from Homo sapiens (Human) (WUGSC:H_DJ0539M06.4 PROTEIN) (E=2.6e⁻¹⁶⁷).

NOV29c is predicted to be expressed in at least Bone, liver, brain, and prostate. This information was derived by determining the tissue sources of the sequences that were included in the invention including but not limited to SeqCalling sources, Public EST sources, Literature sources, and/or RACE sources.

In addition, the sequence is predicted to be expressed in Bone because of the expression pattern of (GENBANK-ID: gb:GENBANK-ID:RNO132352|acc: AJ132352.1) a closely related Rattus norvegicus mRNA for 45 kDa secretory protein, partial homolog.

The disclosed NOV29a polypeptide has homology to the amino acid sequences shown in the BLASTP data listed in Table 29G.

TABLE 29G
BLAST results for NOV29a

| Gene Index/ | | Length | Identity | Positives | |
|---|---|---|---|---|---|
| Identifier | Protein/Organism | (aa) | (%) | (%) | Expect |
| gi\|6624133\|gb\|AAF19259.1\| | similar to 45 kDa | 406 | 387/398 | 390/398 | 0.0 |
| AC004832_4 | secretory protein | | (97%) | (97%) | |
| (AC004832) | [Rattus | | | | |
| | norvegicus]; | | | | |
| | similar to | | | | |
| | CAA10644.1 | | | | |
| | (PID: g4164418) | | | | |
| | [Homo sapiens] | | | | |
| gi\|7110715\|ref\|NP_036561.1\| | SEC14 (S. | 403 | 269/394 | 331/394 | e−165 |
| (NM_012429) | cerevisiae)-like | | (68%) | (83%) | |
| | 2; tocopherol- | | | | |
| | associated | | | | |
| | protein [Homo | | | | |
| | sapiens] | | | | |
| gi\|16758646\|ref\|NP_{—} | SEC14 (S. | 403 | 271/394 | 329/394 | e−164 |
| 446253.1\| | cerevisiae)-like | | (68%) | (82%) | |
| (NM_053801) | 2 [Rattus | | | | |
| | norvegicus] | | | | |
| gi\|13543184\|gb\|AAH05759.1\| | Unknown (protein | 403 | 273/394 | 328/394 | e−164 |
| AAH05759 | for MGC: 6302) | | (69%) | (82%) | |
| (BC005759) | [Mus musculus] | | | | |
| gi\|4164418\|emb\|CAA10644.1\| | 45 kDa secretory | 400 | 267/384 | 326/384 | e−163 |
| (AJ132352) | protein [Rattus | | (69%) | (84%) | |
| | norvegicus] | | | | |

The homology between these and other sequences is shown graphically in the ClustalW analysis shown in Table 29H. In the ClustalW alignment of the NOV29 protein, as well as all other ClustalW analyses herein, the black outlined amino acid residues indicate regions of conserved sequence (i.e., regions that may be required to preserve structural or functional properties), whereas non-highlighted amino acid residues are less conserved and can potentially be altered to a much broader extent without altering protein structure or function.

[image]

[image]

Tables 29I-J list the domain descriptions from DOMAIN analysis results against NOV29. This indicates that the NOV29 sequence has properties similar to those of other proteins known to contain this domain.

TABLE 29I
Domain Analysis of NOV29

gnl|Smart|smart00516, SEC14, Domain in homologues of a S. cerevisiae
phosphatidylinositol transfer protein (Sec14p); Domain in honologues
of a S. cerevisiae phosphatidylinositol transfer protein (Sec14p) and
in RhoGAPs, RhoGEFs and the RasGEF, neurofibromin (NF1). Lipid-binding
domain. The SEC14 domain of Dbl is known to associate with G protein
beta/gamma subunits. (SEQ ID NO:822)
CD-Length = 157 residues, 96.8% aligned
Score = 131 bits (329), Expect =9e−32

| | | | |
|---|---|---|---|
| NOV29: | 90 | VIQLYDSGGLCGYDYEGCPVYFNIIGSLDPKGLLLSASKQDMIRKRIKVCELLLHECELQ | 149 |
| | | \|+\| \|\| \|\|+\|\|\| \| \|\| \|+++++\| +\|\| \| | |
| Sbjct: | 4 | VGKAYIPGGR--YDKDGRPVLVFRAGRFDLK----SVTLEELLRYLVYVLEKALQE---- | 53 |
| NOV29: | 150 | TQKLGRKIEMALMVFDMEGLSLKHLWKPAVEVYQQFFSILEANYPETLKNLIVIRAPKLF | 209 |
| | | +\| \|\| +\|\|++\|\|\|++ \|+\|++ \|\|++\|\|\|\| ++\| \| \| | |
| Sbjct: | 54 | -EKKTGGIEGFTTIFDLKGLSMSN---PDLGVLRKILKILQDHYPERLGKVYIINPPWFF | 109 |
| NOV29: | 210 | PVAFNLVRSFMSEETRRKIVILGDNWKQELTKFISPDQLPVEFGGT 255 | |
| | | \|+++\|\|+\|\|+\|\|\|\| +\|+\|+\|\|++\|\|+\|\|\|\|\|\|\| | |
| Sbjct: | 110 | RVLWKIIKPFLSEKTREKIRFVGPDSKEELLEYIDPEQLPEELGGT 155 | |

TABLE 29J
Domain Analysis of NOV29

gnl|Pfam|pfam00650, CRAL_TRIO, CRAL/TRIO domain.. The original profile
has been extended to include the carboxyl domain from the known
structure of Sec14. (SEQ ID NO:823)
CD-Length = 185 residues, 98.9% aligned
Score = 120 hits (300), Expect =2e−28

| | | | |
|---|---|---|---|
| NOV29: | 73 | RKQQDLDNIV-TWQPPEVVIQLYDSGGLCGYDYEGCPVYFNIIGSLDPKGLLLSASKQDM | 131 |
| | | \|++ +\|\|+ \|+\|\| +\|\|+\|\|\|\| \|\|+\|+\| ++\| | |
| Sbjct: | 3 | RREFGVDTILEEATYPKEVIAKLYPQFIHGSDKOGRPVYLERRGQLNLRKMLFITTVERM | 62 |
| NOV29: | 132 | IRKRIKVCE-LLLHECELQTQKLGRKIEMALMVFDMEGLSL-KHLWKPAVEVYQQFFSIL | 189 |
| | | +\| + \| \|\|+ ++\|+\| \| + \|\|\|++\|+\|+ \|\|\| ++ +\|\| | |
| Sbjct: | 63 | VRNLVYEMEQALLYLLPACSRKVGTLINGSCTVFDLKGVSVSSANWVPGVL--KKVLNIL | 120 |
| NOV29: | 190 | EANYPETLKNLIVIRAPKLFPVAFNLVKSFMSEETRRKIVILGDNWKQELTKFISPDQLP | 249 |
| | | + \|\|\|\| +\|\|\|\|\| +\|+\|\|+ +\|\|\|\|+\|\|+ \|\|\|++\| \|\|\| | |
| Sbjct: | 121 | QDYYPERLGKFYLINAPWLFSTVYKLIKPFLDPKTREKIFVLGNY-KSELLQYIPADNLP | 179 |
| NOV29: | 250 | VEEGGT 255 | |
| | | +\|\|\| | |
| Sbjct: | 180 | AKLGGT 185 | |

Vitamin E (alpha-tocopherol) is an essential dietary nutrient for humans and animals. The mechanisms involved in cellular regulation as well as in the preferential cellular and tissue accumulation of alpha-tocopherol are not yet well established. We previously reported (Stocker, A., Zimmer, S., Spycher, S. E., and Azzi, A. (1999) IUBMB Life 48, 49-55) the identification of a novel 46-kDa tocopherol-associated protein (TAP) in the cytosol of bovine liver. Here, we describe the identification, the molecular cloning into Escherichia coli, and the in vitro expression of the human homologue of bovine TAP, hTAP. This protein appears to belong to a family of hydrophobic ligand binding proteins, which have the CRAL (cis-retinal binding motif) sequence in common. By using a biotinylated alpha-tocopherol derivative and the IASys resonant mirror biosensor, the purified recombinant protein was shown to bind tocopherol at a specific binding site with K(d) 4.6×10(−7) m. Northern analyses showed that hTAP mRNA has a size of approximately 2800 base pairs and is ubiquitously expressed. The highest amounts of hTAP message are found in liver, brain, and prostate. In conclusion, hTAP has sequence homology to proteins containing the CRAL_TRIO structural motif. TAP binds to alpha-tocopherol and biotinylated tocopherol, suggesting the existence of a hydrophobic pocket, possibly analogous to that of SEC14. Zimmer S. et al. (J Biol Chem. Aug. 18, 2000;275(33):25672-80.)

The disclosed NOV29 nucleic acid of the invention encoding a CRAL-TRIO-like protein includes the nucleic acid whose sequence is provided in Table 29A, 29C, 29E or a fragment thereof. The invention also includes a mutant or variant nucleic acid any of whose bases may be changed from the corresponding base shown in Table 29A, 29C, or 29E while still encoding a protein that maintains its CRAL-TRIO-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 26 percent of the bases may be so changed.

The disclosed NOV29 protein of the invention includes the CRAL-TRIO-like protein whose sequence is provided in Table 29B, 29D, or 29F. The invention also includes a mutant or variant protein any of whose residues may be changed from the corresponding residue shown in Table 29B, 29D, or 29F while still encoding a protein that maintains its CRAL-TRIO-like activities and physiological functions, or a functional fragment thereof. In the mutant or variant protein, up to about 32 percent of the residues may be so changed.

The invention further encompasses antibodies and antibody fragments, such as F_{ab} or (F_{ab})₂, that bind immunospecifically to any of the proteins of the invention.

The above disclosed information suggests that this CRAL-TRIO-like protein (NOV29) is a member of a “CRAL-TRIO family”. Therefore, the NOV29 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV29 nucleic acids and proteins of the invention are useful in potential therapeutic applications implicated in brain disorders including epilepsy, eating disorders, schizophrenia, ADD, and cancer; heart disease; inflammation and autoimmune disorders including Crohn's disease, IBD, allergies, rheumatoid and osteoarthritis, inflammatory skin disorders, allergies, blood disorders; psoriasis colon cancer, leukemia AIDS; thalamus disorders; metabolic disorders including diabetes and obesity; lung diseases such as asthma, emphysema, cystic fibrosis, and cancer; pancreatic disorders including pancreatic insufficiency and cancer; and prostate disorders including prostate cancer, and/or other diseases and pathologies.

NOV29 nucleic acids and polypeptides are further useful in the generation of antibodies that bind immuno-specifically to the novel NOV29 substances for use in therapeutic or diagnostic methods. These antibodies may be generated according to methods known in the art, using prediction from hydrophobicity charts, as described in the “Anti-NOVX Antibodies” section below. The disclosed NOV29 protein has multiple hydrophilic regions, each of which can be used as an immunogen. These novel proteins can be used in assay systems for functional analysis of various human disorders, which will help in understanding of pathology of the disease and development of new drug targets for various disorders.

NOV30

A disclosed NOV30 nucleic acid of 717 nucleotides (also referred to as CG56191-01) encoding a novel Ryudocan-like protein is shown in Table 30A. An open reading frame was identified beginning with an ATG initiation codon at nucleotides 22-24 and ending with a TAG codon at nucleotides 658-660. Putative untranslated regions, if any, found upstream from the initiation codon and downstream from the termination codon are underlined in Table 30A, and the start and stop codons are in bold letters.

TABLE 30A
NOV30 Nucleotide Sequence

(SEQ ID NO:127)

CAGGCTGTTCACCCTCTCTGGATGGCGGTACCCACTGCCCCCGCCCTCCTGCTCCTGCTGCTGCTGCTGCT
TTTTGCAGGCACCCCCACCACCCCTGAGTCAATCCAAGAAACTGAGGTCATCAACCCAGGACCGCCTAGGG
GCCCAAACTTCTCCAGATCCCTACTGGAAGACTCTGGATGTGGGTGTTGGGGGCAGGAACCTGATGACTCT
GAGCTCTCTGGCTCTAGAGATATTGATGAGTCAAGGGACCCCAAGATCATCCCTGAAGTGATCCAACCCTT
GGTGCTTCTAGATAACCACATCCCTGAGAGGGCAGGCCCTGGGAACCTGGTCCCCACTGAAACCAAGGAAC
TGGAGGACAACGAGGTCATCCCCAGGAGGATCTCACTCTCTGCGGGGGACCAGGATGTGTCCAATAAGGCA
CCCATGTCCAACACTGCCCAGGGCACCAACATCTTTGAGAGAATGGAGGTCGTGGCAGTCCTGATTGTGGA
CAGCATCGCGGGCATCCTCTCTGCTGTTTTCCTGATCCTGCTTCTGGTGAACCATATGAAGAAGGATGAAG
GCAGAAACGACCTGAGCAGGAAGCCCATCTACAAAAAAGCCCCTAGCAAGGAGTTATTACGCTTCTTCTAT
GAGCACTGGTTTGGACTTTAGGGGATAGGGAAGTCCGAGGATTTTGCAGAGTGGCCATTAGGATGCCGGAG
GACAACC

The NOV30 nucleic acid was identified on chromosome 22 and has 553 of 708 bases (78%) identical to a gb:GENBANK-ID:HUMRYUDO|acc:D13292.1 mRNA from Homo sapiens (mRNA for ryudocan core protein) (E=2.2e⁻⁸²).

A disclosed NOV30 polypeptide (SEQ ID NO:128) encoded by SEQ ID NO:127 is 212 amino acid residues and is presented using the one-letter code in Table 30B. Signal P, Psort and/or Hydropathy results predict that NOV30 contains a signal peptide and is likely to be localized in the plasma membrane with a certainty of 0.4600. The most likely cleavage site for NOV30 is between positions 23 and 24: TPT-TP.

TABLE 30B
Encoded NOV30 protein sequence

(SEQ ID NO:128)

MAVPTAPALLLLLLLLLFAGTPTTPESIQETEVINPGPPRGPNFSRSLLEDSGCGCWGQEPDDSELSGSRDI
DESRDPKIIPEVIQPLVLLDNHIPERAGPGNLVPTETKELEDNEVIPRRISLSAGDQDVSNKAPMSNTAQGS
NIFERMEVVAVLIVDSIAGILSAVFLILLLVNHMKKDEGRNDLSRKPIYKKAPSKELLRFFYEHWFGL

The disclosed NOV30 amino acid sequence has 121 of 198 amino acid residues (61%) identical to, and 140 of 198 amino acid residues (70%) similar to, the 202 amino acid residue ptnr:SWISSPROT-ACC: P34901 protein from Rattus norvegicus (Rat) (Syndecan-4 Precursor (Ryudocan Core Protein)) (E=1.9e⁻⁵¹).

NOV30 is predicted to be expressed in at least myeloid tissue, B-cell lymphoma, including B-cell precursor lymphoblastic leukemia, lymphoplasmacytoid, immunoblastic, lymphocytic/CLL, hairy cell leukemia, large B-cell, mantle-cell, marginal zone and follicular, lymphomas, endothelia, Lymphopoietic and bone marrow (BM) plasma cells (PCs). This information was derived by determining the tissue sources of the sequences that were included in the invention including but not limited to SeqCalling sources, Public EST sources, Literature sources, and/or RACE sources.

In addition, NOV30 is predicted to be expressed in the following tissues because of the expression pattern of(GENBANK-ID: gb:GENBANK-ID:HUMRYUDO|acc: D13292.1) a closely related Human mRNA for ryudocan core protein homolog in species Homo sapiens: myeloid tissue.

NOV30 also has homology to the amino acid sequences shown in the BLASTP data listed in Table 30C.

TABLE 30C
BLAST results for NOV30

| Gene Index/ | | Length | Identity | Positives | |
|---|---|---|---|---|---|
| Identifier | Protein/Organism | (aa) | (%) | (%) | Expect |
| gi\|14771140\|ref\|XP_{—} | syndecan 4 | 198 | 119/197 | 136/197 | 9e−49 |
| 009530.3\| | (amphiglycan, | | (60%) | (68%) | |
| (XM_009530) | ryudocan) [Homo | | | | |
| | sapiens] | | | | |
| gi\|4506861\|ref\|NP_002990.1\| | syndecan 4 | 198 | 120/197 | 137/197 | 2e−45 |
| (NM_002999) | (amphiglycan, | | (60%) | (68%) | |
| | ryudocan) [Homo | | | | |
| | sapiens] | | | | |
| gi\|6981522\|ref\|NP_036781.1\| | ryudocan/syndecan | 202 | 119/199 | 139/199 | 3e−45 |
| (NM_012649) | 4 [Rattus | | (59%) | (69%) | |
| | norvegicus] | | | | |
| gi\|6755442\|ref\|NP_035651.1\| | syndecan 4 [Mus | 198 | 117/199 | 136/199 | 6e−41 |
| (NM_011521) | musculus] | | (58%) | (67%) | |
| gi\|1351051\|sp\|P49416\| | SYNDECAN-4 | 197 | 80/216 | 105/216 | 1e−14 |
| SDC4_CHICK | PRECURSOR | | (37%) | (48%) | |

The homology of these sequences is shown graphically in the ClustalW analysis shown in Table 30D.

[image]

[image]

Table 30E lists the domain description from DOMAIN analysis results against NOV30. This indicates that the NOV30 sequence has properties similar to those of other proteins known to contain this domain.

TABLE 30E
Domain Analysis of NOV30

gnl|Pfam|pfam01034, Syndecan, Syndecan domain. Syndecans are
transmembrane heparin sulfate proteoglycans which are implicated in
the binding of extracellular matrix components and growth factors (SEQ
ID NO:824)
CD-Length = 359 residues, 21.7% aligned
Score = 41.6 bits (96), Expect = 5e−05

| | | | |
|---|---|---|---|
| NOV30: | 115 | NEVIPRRISLSAGDQDVSNKAPMSNTA--------QGSNIFERNEVVAVLIVDSIAGILS | 166 |
| | | \|\| \| ++ + +\|+\|\| \| \|\|\|\|\|+\|+\| +\|+\| | |
| Sbjct: | 258 | NETSPENTAAANPEPLGRGQRPIDNTVDSGSSGAQQSQKILERKEVLAAVIAGGVVGLLF | 317 |
| NOV30: | 167 | AVFLILLLVNHM-KKDEG 183 | |
| | | \|\|\|\|++++ \|\|\|\|\|\| | |
| Sbjct: | 318 | AVFLVMFMLYRMKKKDEG 335 | |

Kininogens, the high molecular weight precursor of vasoactive kinins, bind to a wide variety of cells in a specific, reversible, and saturable manner. The cell docking sites have been mapped to domains D3 and D5(H) of kininogens; however, the corresponding cellular acceptor sites are not fully established. To characterize the major cell binding sites for kininogens exposed by the endothelial cell line EA.hy926, intact cells were digested with trypsin and other proteases and found a time- and concentration-dependent loss of (125)I-labeled high molecular weight kininogen (H-kininogen) binding capacity (up to 82%), indicating that proteins are crucially involved in kininogen cell attachment. Cell surface digestion with heparinases similarly reduced kininogen binding capacity (up to 78%), and the combined action of heparinases and trypsin almost eliminated kininogen binding (up to 85%), suggesting that proteoglycans of the heparan sulfate type are intimately involved. Consistently, inhibitors such as p-nitrophenyl-beta-d-xylopyranoside and chlorate interfering with heparan sulfate proteoglycan biosynthesis reduced the total number of kininogen binding sites in a time- and concentration-dependent manner (up to 67%). In vitro binding studies demonstrated that biotinylated H-kininogen binds to heparan sulfate glycosaminoglycans via domains D3 and D5(H) and that the presence of Zn(2+) promotes this association. Cloning and over-expression of the major endothelial heparan sulfate-type proteoglycans syndecan-1, syndecan-2, syndecan-4, and glypican in HEK293t cells significantly increased total heparan sulfate at the cell surface and thus the number of kininogen binding sites (up to 3.3-fold). This gain in kininogen binding capacity was completely abolished by treating transfected cells with heparinases. It was concluded that heparan sulfate proteoglycans on the surface of endothelial cells provide a platform for the local accumulation of kininogens on the vascular lining. This accumulation may allow the circumscribed release of short-lived kinins from their precursor molecules in close proximity to their sites of action (Renne et al., J Biol Chem 2000, 275(43):33688-96).

Lymphopoietic cells require interactions with bone marrow stroma for normal maturation and show changes in adhesion to matrix during their differentiation. Syndecan, a heparan sulfate-rich integral membrane proteoglycan, functions as a matrix receptor by binding cells to interstitial collagens, fibronectin, and thrombospondin. Therefore, it was asked whether syndecan was present on the surface of lymphopoietic cells. In bone marrow, syndecan was only found on precursor B cells. Expression changes with pre-B cell maturation in the marrow and with B-lymphocyte differentiation to plasma cells in interstitial matrices. Syndecan on B cell precursors is more heterogeneous and slightly larger than on plasma cells. Syndecan 1) is lost immediately before maturation and release of B lymphocytes into the circulation, 2) is absent on circulating and peripheral B lymphocytes, and 3) is reexpressed upon their differentiation into immobilized plasma cells. Thus, syndecan is expressed only when and where B lymphocytes associate with extracellular matrix. These results indicate that B cells differentiating in vivo alter their matrix receptor expression and suggest a role for syndecan in B cell stage-specific adhesion (Sanderson et al., Cell Regul 1989,1(1):27-35).

Detection of abnormal numbers and/or distribution of bone marrow (BM) plasma cells (PCs) on trephine biopsies can be important in the differential diagnosis of multiple myeloma (MM) and other PC disorders. A variety of immunohistochemical markers can potentially improve the specificity and sensitivity of PC detection on routine histological sections obtained from trephine BM biopsies, but most of them are not completely satisfactory. In one study, the antibody CD138/B-B4, which is an optimal marker for PC detection on BM aspirates by flow cytometry, was investigated to determine whether it can be used successfully for the identification of PCs also on formalin-fixed, decalcified biopsies. A series of samples including normal BM, MM, monoclonal gammopathies of undetermined significance, and B-cell lymphoma of various types, including B-cell precursor lymphoblastic leukemia, lymphoplasmacytoid, immunoblastic, lymphocytic/CLL, hairy cell leukemia, large B-cell, mantle-cell, marginal zone and follicular lymphomas, have been investigated for CD138 expression using a sensitive immunohistochemical technique. Within the BM microenvironment, CD138 was characterized by excellent sensitivity and specificity. Virtually all normal and neoplastic PCs expressed clear-cut membrane CD138 immunostaining, whereas all other cell types did not. All cases of MM, including plasmablastic and leukemic cases, showed strong immunoreactivity. Conversely, all B-cell lymphomas, including all cases characterized by secretive features, lymphoplasmacytoid, and immunoblastic lymphomas, were completely negative. These results demonstrate that CD138 is a highly sensitive and specific marker that is useful for the rapid and precise localization of normal and neoplastic PCs on routine BM sections. In addition, because of its clear-cut cell membrane localization, CD138 can be used successfully in double-marker immunostaining reactions to evaluate precisely nuclear prognostic markers such as Ki67 and p53 in MMs (Chilosi et al., Mod Pathol 1999, 12(12):1101-6).

Monoclonal antibody therapy has emerged as a viable treatment option for patients with lymphoma and some leukemias. It is now beginning to be investigated for treatment of multiple myeloma. There are relatively few surface antigens on the plasma cells that are suitable for antibody-directed treatment. Possible molecules include HM1.24, CD38, ICAM-1 (CD54), CD40, CD45, CD20, and syndecan 1. There is now some clinical experience with anti-CD38 antibody in lymphoma and myeloma. However, to date, there has been minimal clinical activity observed. Additional antibodies are entering clinical trials. A new approach involves the generation of an anti-CD38 single-chain variable fragment (scFv) construct that acts as the carrier of a toxin gene instead of being conjugated directly to the toxin itself. It is hoped that expression of the toxin by CD38+ plasma cells will promote suicide of the malignant cells without affecting normal cells or generating an immunologic response to the toxin. Ongoing clinical trials are also attempting to target B-cell antigens such as CD20. Although CD20 is present only on 20% of myeloma cells, it may be present on myeloma precursor cells. This treatment has met with success in follicular lymphoma and is now being evaluated in clinical trials in both Europe and the United States for myeloma. Although these clinical trials are in very early stages, researchers are beginning to understand that antibody therapy can be used not only as a carrier molecule of radioisotopes and toxins, but also as molecules that can trigger tumor cells and promote growth arrest or apoptosis (Maloney et al., Semin Hematol 1999, 36(1 Suppl 3):30-3).

The NOV30 nucleic acid of the invention encoding a Ryudocan-like protein includes the nucleic acid whose sequence is provided in Table 30A, or a fragment thereof. The invention also includes a mutant or variant nucleic acid any of whose bases may be changed from the corresponding base shown in Table 30A while still encoding a protein that maintains its Ryudocan-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 22% of the residues may be so changed.

The NOV30 protein of the invention includes the Ryudocan-like protein whose sequence is provided in Table 30B. The invention also includes a mutant or variant protein any of whose residues may be changed from the corresponding residue shown in Table 30B while still encoding a protein that maintains its Ryudocan-like activities and physiological functions, or a functional fragment thereof. In the mutant or variant protein, up to about 63 of the bases may be so changed.

The NOV30 nucleic acids and proteins of the invention are useful in potential diagnostic and therapeutic applications implicated in various diseases and disorders described below and/or other pathologies. For example, the compositions of the present invention will have efficacy for treatment of patients suffering from: brain disorders including epilepsy, eating disorders, schizophrenia, ADD, cancer, heart disease, inflammation and autoimmune disorders including Crohn's disease, IBD, allergies, rheumatoid and osteoarthritis, inflammatory skin disorders, allergies, blood disorders, psoriasis, colon cancer, leukemia, AIDS, thalamus disorders, metabolic disorders including diabetes and obesity, lung diseases such as asthma, myelomas, emphysema, cystic fibrosis, and cancer, pancreatic disorders including pancreatic insufficiency and cancer, and prostate disorders including prostate cancer and other diseases, disorders and conditions of the like.

NOV30 nucleic acids and polypeptides are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. For example the disclosed NOV30 protein have multiple hydrophilic regions, each of which can be used as an immunogen. This novel protein also has value in development of powerful assay system for functional analysis of various human disorders, which will help in understanding of pathology of the disease and development of new drug targets for various disorders.

NOV31

A disclosed NOV31 nucleic acid of 683 nucleotides (also referred to as CG56392-01) encoding a novel Sulfur-rich Keratin-like protein is shown in Table 31A. An open reading frame was identified beginning with an ATG initiation codon at nucleotides 46-48 and ending with a TGA codon at nucleotides 652-654. Putative untranslated regions, if any, found upstream from the initiation codon and downstream from the termination codon are underlined in Table 31 A, and the start and stop codons are in bold letters.

TABLE 31K
NOV31 Nucleotide Sequence

(SEQ ID NO:129)

GAGCTGTGTAACAGCAACCGGAAAGAGAAACAATGGTGTGTTCCTATGTGGGATATAAAGAGCCGGGGCTC
AGGGGGCTCCACACCTGCACCTCCTTCTCACCTGCTCCTCTACCTGCTCCACCCTCAATCCACCAGAACCA
TGGGCTGCTGTGGCTGCTCCGGAGGCTGTGGCTCCAGCTGTGGACGCTGTGACTCCAGCTGTGGGAGCTGT
GGCTCTGGCTGCAGGGGCTGTGCCCCCAGCTGCTCTGCACCCGTCTACTGCTGCAAGCCCGTGTGCTGCTG
TGTTCCAGCCTGTTCCTGCTCTAGCTGTGGCAAGCGGGCCTGTGGCTCCTGTGGGGGCTCCAAGGGAGGCT
GTGGTTCTTGTGGCTGCTCCCAGTGCAGTTGCTGCAACCCCTGCTGTTGCTCTTCAGGCTGTGGGTCATCC
TGCTGCCAGTGCAGCTGCTGCAAGCCCTACTGCTCCCAGTCCAGCTGTTGTAAGCCCTGTTGCTGCTCCTC
AGGCTGTGGATCATCCTGCTGCCAGTCCAGCTGCTGCAAGCCCTCCTGCTGCCAGTCCAGCTGCTGTGTCC
CCGTGTGCTCCCAGTCCAGCTGCTGCAAGCCCTGTTGCTGCCAGTCCAACTGTTGTGTCCCTGTGTGCTGC
CAGTGTAAGATCTGAGGCTCTAGTGGGAAACCTCAGGTAGCTCC

The NOV31 nucleic acid was identified on chromosome 11 and has 654 of 683 bases (95%) identical to a gb:GENBANK-ID:HSA6693|acc:AJ006693.1 mRNA from Homo sapiens (UHS KerA gene) (E=3.3e⁻¹³⁶).

A disclosed NOV31 polypeptide (SEQ ID NO:130) encoded by SEQ ID NO:129 is 202 amino acid residues and is presented using the one-letter code in Table 31B. Signal P, Psort and/or Hydropathy results predict that NOV31 contains a signal peptide and is likely to be localized to the cytoplasm with a certainty of 0.4500. The most likely cleavage site for a NOV31 peptide is between amino acids 32 and 33: TRT-MG.

TABLE 31B
Encoded NOV31 protein sequence

(SEQ ID NO:130)

MWDIKSRGSGGSTPAPPSHLLLYLLHPQSTRTMCCCGCSGGCGSSCGGCDSSCGSCGSGCRGCGPSCCAPVY
CCKPVCCCVPACSCSSCGKRGCGSCGGSKGGCGSCGCSQCSCCKPCCCSSGCGSSCCQCSCCKPYCSQSSCC
KPCCCSSGCGSSCCQSSCCKPCCCQSSCCVPVCCQSSCCKPCCCQSNCCVPVCCQCKI

The disclosed NOV31 amino acid sequence has 158 of 170 amino acid residues (92%) identical to, and 158 of 170 amino acid residues (92%) similar to, the 169 amino acid residue ptnr:SWISSNEW-ACC:P26371 protein from Homo sapiens (Human) (Keratin, Ultra High-Sulfur Matrix Protein A (Uhs Keratin A) (Uhs Kera)) (E=1.8e⁻¹⁰¹).

NOV31 is predicted to be expressed in at least Kidney, Pancreas, Testis and Whole Organism. This information was derived by determining the tissue sources of the sequences that were included in the invention including but not limited to SeqCalling sources, Public EST sources, Literature sources, and/or RACE sources.

In addition, NOV31 is predicted to be expressed in the following tissues because of the expression pattern of (GENBANK-ID:gb:GENBANK-ID:HSA6693|acc:AJ006693.1) a closely related Homo sapiens UHS KerA gene homolog in species Homo sapiens: Kidney, Pancreas and Testis.

NOV31 also has homology to the amino acid sequences shown in the BLASTP data listed in Table 31C.

TABLE 31C
BLAST results for NOV31

| Gene Index/ | | Length | Identity | Positives | |
|---|---|---|---|---|---|
| Identifier | Protein/Organism | (aa) | (%) | (%) | Expect |
| gi\|12835376\|dbj\|BAB23238.1\| | data source: SPTR, | 195 | 76/157 | 91/157 | 5e−13 |
| (AK004258) | source | | (48%) | (57%) | |
| | key: Q64526, | | | | |
| | evidence: ISS˜putative˜ | | | | |
| | similar to | | | | |
| | ULTRA-HIGH | | | | |
| | SULPHUR KERATIN | | | | |
| | [Mus musculus] | | | | |
| gi\|2136964\|pir\|\|I46489 | cysteine-rich | 126 | 53/120 | 72/120 | 3e−11 |
| | hair keratin | | (44%) | (59%) | |
| | associated | | | | |
| | protein - rabbit | | | | |
| gi\|12844600\|dbj\|BAB26426.1\| | data source: SPTR, | 168 | 59/116 | 70/116 | 1e−10 |
| (AK009665) | source | | (50%) | (59%) | |
| | key: Q28707, | | | | |
| | evidence: ISS˜homolog | | | | |
| | to CYSTEINE | | | | |
| | RICH HAIR KERATIN | | | | |
| | ASSOCIATED | | | | |
| | PROTEIN˜putative | | | | |
| | [Mus musculus] | | | | |
| gi\|15082220\|ref\|NP_{—} | keratin | 195 | 56/122 | 65/122 | 2e−10 |
| 149048.1\| | associated | | (45%) | (52%) | |
| (NM_033059) | protein 4.14 | | | | |
| | [Homo sapiens] | | | | |
| gi\|13386198\|ref\|NP_{—} | RIKEN cDNA | 165 | 53/106 | 61/106 | 2e−10 |
| 081363.1\| | 2300006N05 [Mus | | (50%) | (57%) | |
| (NM_027087) | musculus] | | | | |

The homology of these sequences is shown graphically in the ClustalW analysis shown in Table 31D.

[image]

[image]

Insulin-like growth factor 1 (IGF-1) mediates many of the actions of growth hormone. Overexpression of IGF-1 has been reported to have endocrine and paracrine/autocrine effects on somatic growth in transgenic mice. To study the paracrine/autocrine effects of IGF-1 in hair follicles, transgenic mice were produced by pronuclear microinjection of a construct containing a mouse ultra-high sulfur keratin (UHS-KER) promoter linked to an ovine IGF-1 cDNA. This UHS-KER promoter has previously been shown to direct expression of a reporter gene to the hair follicles of transgenic mice. Four transgenic mouse lines were established as a result of microinjection of 435 embryos. Transgene expression was found in skin at day 8 and day 15 of age in three of the lines. Progeny tests were carried out by mating two of the transgenic expressing males to nontransgenic females. Mice from one line were all nonexpressors while four of the 12 mice from the other showed integration of the transgene and three expressed transgene IGF-1 mRNA in the skin. Vibrissa growth at 11-21 d of age was significantly greater in transgenic expressors than in their nontransgenic littermates. Specifically, the increase in vibrissa length for transgenics at days 11-16 (20.5%) is approximately 2-fold compared with days 16-21 (11.9%). These results demonstrate that local overexpression of IGF-1 in transgenic mice is capable of stimulating vibrissa growth during the first neonatal hair cycle (Su et al., J Invest Dermatol 1999, 112(2):245-8).

The major histological components of the hair follicle are the hair cortex and cuticle. The hair cuticle cells encase and protect the cortex and undergo a different developmental program to that of the cortex. In one study, the molecular characterization of a set of evolutionarily conserved hair genes which are transcribed in the hair cuticle late in follicle development was reported. Two genes were isolated and characterized, one expressed in the human follicle and one in the sheep follicle. Each gene encodes a small protein of 16 kD, containing greater than 50 cysteine residues, ranging from 31 to 36 mol % cysteine. Their high cysteine content and in vitro expression data identify them as ultra-high-sulfur (UHS) keratin proteins. The predicted proteins are composed almost entirely of cysteine-rich and glycine-rich repeats. Genomic blots reveal that the UHS keratin proteins are encoded by related multigene families in both the human and sheep genomes. Tissue in situ hybridization demonstrates that the expression of both genes is localized to the hair fiber cuticle and occurs at a late stage in fiber morphogenesis (MacKinnon et al., J Cell Biol 1990, 111(6 Pt 1):2587-600).

The NOV31 nucleic acid of the invention encoding a Sulfur-rich Keratin-like protein includes the nucleic acid whose sequence is provided in Table 31A, or a fragment thereof. The invention also includes a mutant or variant nucleic acid any of whose bases may be changed from the corresponding base shown in Table 31A while still encoding a protein that maintains its Sulfur-rich Keratin-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of non-limiting example, modified bases, and nucleic acids whosesugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 5% of the residues may be so changed.

The NOV31 protein of the invention includes the Sulfur-rich Keratin-like protein whose sequence is provided in Table 31B. The invention also includes a mutant or variant protein any of whose residues may be changed from the corresponding residue shown in Table 31B while still encoding a protein that maintains its Sulfur-rich Keratin-like activities and physiological functions, or a functional fragment thereof. In the mutant or variant protein, up to about 56% of the bases may be so changed.

The NOV31 nucleic acids and proteins of the invention are useful in potential diagnostic and therapeutic applications implicated in various diseases and disorders described below and/or other pathologies. For example, the compositions of the present invention will have efficacy for treatment of patients suffering from: brain disorders including epilepsy, eating disorders, schizophrenia, ADD, cancer, heart disease, inflammation and autoimmune disorders including Crohn's disease, IBD, allergies, rheumatoid and osteoarthritis, inflammatory skin disorders, allergies, blood disorders, psoriasis, colon cancer, leukemia, AIDS, thalamus disorders, metabolic disorders including diabetes and obesity, lung diseases such as asthma, emphysema, cystic fibrosis, and cancer, pancreatic disorders including pancreatic insufficiency and cancer, and prostate disorders including prostate cancer and other diseases, disorders and conditions of the like.

NOV31 nucleic acids and polypeptides are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. For example the disclosed NOV31 protein have multiple hydrophilic regions, each of which can be used as an immunogen. This novel protein also has value in development of powerful assay system for functional analysis of various human disorders, which will help in understanding of pathology of the disease and development of new drug targets for various disorders.

NOV32

A disclosed NOV32 nucleic acid of 1575 nucleotides (also referred to as CG56686-01) encoding a novel DNMT1 associated protein-1 (DMAP)-like protein is shown in Table 32A. An open reading frame was identified beginning with an ATG initiation codon at nucleotides 94-96 and ending with a TGA codon at nucleotides 1573-1575. Putative untranslated regions, if any, found upstream from the initiation codon and downstream from the termination codon are underlined in Table 32A, and the start and stop codons are in bold letters.

TABLE 32A
NOV32 Nucleotide Sequence

(SEQ ID NO:131)

CTTGGAGGCTGCAGGTCCGGACCCAGGTGCGGAAGTGCGAGGGCCCAGGCACTGACCCTTGACCTCCGGTG
GCTCCCCCATCTCTCAGGCGCGATGGCTACGGGCGCGGATGTACGGGACATTCTAGAACTCGGGCGTCCAG
AAGGGGATGCAGCCTCTGGGACCATCAGCAAGAAGGACATTATCAACCCGGACAAGAAAAAATCCAAGAAG
TCCTCTCAGACACTGACTTTCAAGAGGCCCGAGGGCATGCACCCGGAAGTCTATGCCTTGCTCTACTCTGA
CAACAACAAGGGCTCCTGCTTGCTTAGCAGGATGCAGGAGGACCTGAAGTCTTTTGCTCCAGGACATGACT
TTCTTGCTATAGGGGATGCACCCCCACTGCTACCCAGTGACACTGGCCAGGGATACCGTACAGTGAAGGCC
AAGTTGGGCTCCAAGAAGGTGCGGCCTTGGAAGTGGATGCCATTCACCAACCCGGCCCGCAAGGACCGAGC
AATGTTCTTCCACTGGCGACGTGCAGCGGAGGAGGGCAAGGACTACCCCTTTGCCAGGTTCAATAAGACTG
TGCAGGTGCCTGTGTACTCGGAGCAGGAGTACCAGCTTTATCTCCACGATGATGCTTGGACTAAGGCAGAA
ACTCACCACCTCTTTGACCTCAGCCGCCGCTTTGACCTGCGTTTTGTTGTTATCCATGACCGGTATGACCA
CCACCAGTTCAAGAAGCGTTCTGTGGAAGACCTGAAGGAGCGGTACTACCACATCTGTGCTAAGCTTGCCA
ACGTGCGGGCTGTGCCAGGCACAGACCTTAAGATACCAGTATTTGATGCTGGGCACGAACGACGGCGGAAG
GAACAGCTTGAGCGTCTCTACAACCGGACCCCAGAGCAGGTGGCAGAGGAGGAGTACCTGCTACAGGAGCT
GCGCAAGATTGAGGCCCGGAAGAAGGAGCGGGAGAAACGCAGCCAGCACCTGCAGAAGCTGATCACAGCGG
CAGACACCACTGCAGAGCAGCGGCGCACGGAACGCAAGGCCCCCAAAAAGAAGCTACCCCAGAAAAAGGAG
GCTCAGAAGCCGGCTGTTCCTGAGACTGCAGGCATCAAGTTTCCAGACTTCAAGTCTGCAGGTGTCACGCT
GCGGAGCCAACGGATGAAGCTGCCAAGCTCTGTGGGACAGAAGAAGATCAAGGCCCTGGAACAGATGCTGC
TGGAGCTTGGTGTGGAGCTGAGCCCGACACCTACGGAGCAGCTGGTGCACATGTTCAATGAGCTGCGAAGC
CACCTGGTGCTGCTCTACGAGCTCAAGCAGGCCTGTGCCAACTGCGAGTATGAGCTGCAGATGCTGCGGCA
CCGTCATGACGCACTGGCCCGGGCTGGTGTGCTAGGGGGCCCTGCCACACCAGCATCAGGCCCAGGCCCGG
CCTCTGCTGAGCCGGCAGTGACTGAACCCGGACTTGGTCCTGACCCCAAGGACACCATCATTCATGTGGTG
GGCGCACCCCTCACGCCCAATTCGAGAAAGCGACGGGAGTCGGCCTCCAGCTCATCTTCCGTGAAGAAAGC
CAAGAAGCCGTGA

The NOV32 nucleic acid was identified on chromosome 1p34 and has 1244 of 1273 bases (97%) identical to a gb:GENBANK-ID:AF265228|acc:AF265228.1 mRNA from Homo sapiens (DNMT1 associated protein-1 (DMAP1) mRNA, complete cds) (E=1.0e⁻³⁰⁹).

A disclosed NOV32 polypeptide (SEQ ID NO:132) encoded by SEQ ID NO:131 is 493 amino acid residues and is presented using the one-letter code in Table 32B. Signal P, Psort and/or Hydropathy results predict that NOV32 does not contain a signal peptide and is likely to be localized to the nucleus with a certainty of 0.9800.

TABLE 32B
Encoded NOV32 protein sequence

(SEQ ID NO:132)

MATGADVRDILELGGPEGDAASGTISKKDIINPDKKKSKKSSETLTFKRPEGMHREVYALLYSDKNKGSCLL
SRMQEDLKSFAPGHDFLAIGDAPPLLPSDTGQGYRTVKAKLGSKKVRPWKWMPFTNPARKDGANFFHWRRAA
EEGKDYPFARFNKTVQVPVYSEQEYQLYLHDDAWTKAETDHLFDLSRRFDLRFVVIHDRYDHQQFKKRSVED
LKERYYHICAKLANVRAVPGTDLKIPVFDAGHERRRKEQLERLYNRTPEQVAEEEYLLQELRKIEARKKERE
KRSQDLQRLITAADTTAEQRRTERKAPKKKLPQKKEAEKPAVPETAGIKFPDFKSAGVTLRSQRMKLPSSVG
QKKIKALEQMLLELGVELSPTPTEELVHMFNELRSDLVLLYELKQACANCEYELQMLRHRHEALARAGVLGG
PATPASGPGPASAEPAVTEPGLGPDPKDTIIDVVGAPLTPNSRKRRESASSSSSVKKAKKP

The disclosed NOV32 amino acid sequence has 401 of 401 amino acid residues (100%) identical to, and 401 of 401 amino acid residues (100%) similar to, the 467 amino acid residue ptnr:SPTREMBL-ACC:Q9NPF5 protein from Homo sapiens (Human) (Hypothetical 53.0 Kda Protein (Dnmt1 Associated Protein-1) (E=1.3e⁻²⁴⁸).

NOV32 is predicted to be expressed in at least Adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea and uterus. This information was derived by determining the tissue sources of the sequences that were included in the invention including but not limited to SeqCalling sources, Public EST sources, Literature sources, and/or RACE sources.

NOV32 also has homology to the amino acid sequences shown in the BLASTP data listed in Table 32C.

TABLE 32C
BLAST results for NOV32

| Gene Index/ | | Length | Identity | Positives | |
|---|---|---|---|---|---|
| Identifier | Protein/Organism | (aa) | (%) | (%) | Expect |
| gi\|7243231\|dbj\|BAA92663.1\| | KIAA1425 protein | 495 | 446/473 | 446/473 | 0.0 |
| (AB037846) | [Homo sapiens] | | (94%) | (94%) | |
| gi\|13123776\|ref\|NP_{—} | DNA | 467 | 446/473 | 446/473 | 0.0 |
| 061973.1\| | methyltransferase | | (94%) | (94%) | |
| (NM_019100) | 1-associated | | | | |
| | protein 1 [Homo | | | | |
| | sapiens] | | | | |
| gi\|12052838\|emb\|CAB66592.1\| | hypothetical | 467 | 443/473 | 445/473 | 0.0 |
| (AL136657) | protein [Homo | | (93%) | (93%) | |
| | sapiens] | | | | |
| gi\|12963557\|ref\|NP_{—} | DNMT1 associated | 468 | 437/474 | 438/474 | 0.0 |
| 075667.1\| | protein-1 [Mus | | (92%) | (92%) | |
| (NM_023178) | musculus] | | | | |
| gi\|12805675\|gb\|AAH02321.1\| | Unknown (protein | 451 | 420/457 | 421/457 | 0.0 |
| AAH02321 | for | | (91%) | (91%) | |
| (BC002321) | IMAGE: 3594236) | | | | |
| | [Mus musculus] | | | | |

The homology of these sequences is shown graphically in the ClustalW analysis shown in Table 32D.

[image]

[image]

Methylation of CpG islands is associated with transcriptional silencing and the formation of nuclease-resistant chromatin structures enriched in hypoacetylated histones. Methyl-CpG-binding proteins, such as MeCP2, provide a link between methylated DNA and hypoacetylated histones by recruiting histone deacetylase, but the mechanisms establishing the methylation patterns themselves are unknown. Whether DNA methylation is always causal for the assembly of repressive chromatin or whether features of transcriptionally silent chromatin might target methyltransferase remains unresolved. Mammalian DNA methyltransferases (DNMT) show little sequence specificity in vitro, yet methylation can be targeted in vivo within chromosomes to repetitive elements, centromeres and imprinted loci. This targeting is frequently disrupted in tumour cells, resulting in the improper silencing of tumour-suppressor genes associated with CpG islands. Robertson et al. (Nat Genet 2000, 25:338-42) have shown that the predominant mammalian DNA methyltransferase, DNMT1, co-purifies with the retinoblastoma (Rb) tumour suppressor gene product, E2F1, and HDAC1 and that DNMT1 cooperates with Rb to repress transcription from promoters containing E2F-binding sites. These results establish a link between DNA methylation, histone deacetylase and sequence-specific DNA binding activity, as well as a growth-regulatory pathway that is disrupted in nearly all cancer cells. Recently, Rountree et al. (Nat Genet, 2000, 25:269-77) have shown that the non-catalytic amino terminus of DNMT1 binds to HDAC2 and a new protein, DMAP1 (for DNMT1 associated protein), and can mediate transcriptional repression. DMAP1 has intrinsic transcription repressive activity, and binds to the transcriptional co-repressor TSG101. DMAP1 is targeted to replication foci through interaction with the far N terminus of DNMT1 throughout S phase, whereas HDAC2 joins DNMT1 and DMAP1 only during late S phase, providing a platform for how histones may become deacetylated in heterochromatin following replication. Thus, DNMT1 not only maintains DNA methylation, but also may directly target, in a heritable manner, transcriptionally repressive chromatin to the genome during DNA replication.

The NOV32 nucleic acid of the invention encoding a DNMT1 associated protein-1 (DMAP)-like protein includes the nucleic acid whose sequence is provided in Table 32A, or a fragment thereof. The invention also includes a mutant or variant nucleic acid any of whose bases may be changed from the corresponding base shown in Table 32A while still encoding a protein that maintains its DNMT1 associated protein-1 (DMAP)-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 3% of the residues may be so changed.

The NOV32 protein of the invention includes the DNMT1 associated protein-1 (DMAP)-like protein whose sequence is provided in Table 32B. The invention also includes a mutant or variant protein any of whose residues may be changed from the corresponding residue shown in Table 32B while still encoding a protein that maintains its DNMT1 associated protein-1 (DMAP)-like activities and physiological functions, or a functional fragment thereof. In the mutant or variant protein, up to about 9% of the bases may be so changed.

The NOV32 nucleic acids and proteins of the invention are useful in potential diagnostic and therapeutic applications implicated in various diseases and disorders described below and/or other pathologies. For example, the compositions of the present invention will have efficacy for treatment of patients suffering from: cancers such as breast cancer, colorectal cancers, lung cancer, liver cancer, pancreatic cancer, prostate cancer, stomach cancers, developmental syndromes, Fragile X and Rett and other diseases, disorders and conditions of the like.

NOV32 nucleic acids and polypeptides are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. For example the disclosed NOV32 protein have multiple hydrophilic regions, each of which can be used as an immunogen. This novel protein also has value in development of powerful assay system for functional analysis of various human disorders, which will help in understanding of pathology of the disease and development of new drug targets for various disorders.

NOV33

A disclosed NOV33 nucleic acid of 7693 nucleotides (also referred to as CG56688-01) encoding a novel Notch1-like protein is shown in Table 33A. An open reading frame was identified beginning with an ATG initiation codon at nucleotides 1-3 and ending with a TAA codon at nucleotides 7669-7671. Putative untranslated regions, if any, found upstream from the initiation codon and downstream from the termination codon are underlined in Table 33A, and the start and stop codons are in bold letters.

TABLE 33A
NOV33 Nucleotide Sequence

(SEQ ID NO:133)

ATGCCGCCGCTCCTGGCGCCCCTGCTCTGCCTGGCGCTGCTGCCCGCGCTCGCCGCACGAGGCCCGCGATG
CTCCCAGCCCGGTGAGACCTGCCTGAATGGCGGGAAGTGTGAAGCGGCCAATGGCACGGAGGCCTGCGTCT
GTGGCGGGGCCTTCGTGGGCCCGCGATGCCAGGACCCCAACCCGTGCCTCAGCACCCCCTGCAAGAACGCC
GGGACATGCCACGTGGTGGACCGCAGAGGCGTGGCAGACTATGCCTGCAGCTGTGCCCTGGGCTTCTCTGG
GCCCCTCTGCCTGACACCCCTGGACAACGCCTCCCTCACCAACCCCTGCCGCAACGGGGGCACCTGCGACC
TGCTCACGCTGACGGAGTACAAGTGCCGCTGCCCGCCCGCCTGGTCAGGGAAATCGTGCCAGCAGGCTCAC
CCGTGCGCCTCCAACCCCTGCGCCAACGGTGGCCAGTGCCTGCCCTTCGAGGCCTCCTACATCTGCCACTG
CCCACCCAGCTTCCATGGCCCCACCTGCCGGCAGGATGTCAACGAGTGTGCCCAGAAGCCCCGGCTTTGCC
GCCACGGAGGCACCTGCCACAACGAGGTCGGCTCCTACCGCTGCGTCTGCCGCGCCACCCACACTGGCCCC
AACTGCGAGCGGCCCTACGTGCCCTGCACCCCCTCGCCCTGCCAGAACGGGGGCACCTGCCGCCCCACGCG
CGACGTCACCCACGAGTGTGCCTGCCTGCCAGGCTTCACCGGCCAGAACTGTGAGGAAAATATCGACGATT
GTCCAGGAAACAACTGCAAGAACGGGGGTGCCTGTGTGGACGGCGTGAACACCTACAACTGCCCGTGCCCG
CCAGAGTGGACAGGTCAGTACTGTACCGAGGATGTGGACGAGTGCCAGCTGATCCCAAATGCCTGCCAGAA
CGGCGGGACCTGCCACAACACCCACGGTGGCTACAACTGCGTGTGTGTCAACGGCTGGACTGGTGAGGACT
GCAGCGAGAACATTGATGACTGTGCCAGCGCCGCCTGCTTCCACGGCGCCACCTCCCATGACCGTGTGGCC
TCCTTTTACTGCGAGTGTCCCCATGGCCGCACAGGTCTGCTGTGCCACCTCAACGACGCATGCATCAGCAA
CCCCTGTAACGAGGGCTCCAACTGCGACACCAACCCTGTCAATGCCAAGGCCATCTGCACCTGCCCCTCGG
GGTACACGGGCCCGGCCTGCACCCAGGACGTGGATGAGTGCTCGCTGGCTGCCAACCCCTGCGAGCATCCG
GCCAAGTGCATCAACACGCTGGGCTCCTTCGAGTGCCAGTGTCTGCAGGGCTACACGGGCCCCCGATGCGA
GATCGACGTCAACGAGTGCGTCTCGAACCCGTGCCAGAACGACCCCACCTGCCTGGACCAGATTGGGGAGT
TCCAGTGCATGTGCATGCCCGGCTACGAGGGTGTGCACTGCGAGGTCAACACAGACGAGTGTGCCAGCAGC
CCCTGCCTGCACAATGGCCGCTGCCTGGACAAGATCAATGAGTTCCAGTGCGAGTGCCCCACCGGCTTCAC
TGGGCATCTGTGCCAGTACGATGTGGACCAGTGTGCCAGCACCCCCTGCAAGAATGGTGCCAAGTGCCTGG
ACGGACCCAACACTTACACCTGTGTGTGCACGGAAGOGTACACGGGGACGCACTGCGAGGTGGACATCGAT
GAGTGCGACCCCGACCCCTGCCACTACGGCTCCTGCAAGGACGGCGTCGCCACCTTCACCTGCCTCTGCCG
CCCAGGCTACACGGGCCACCACTGCGAGACCAACATCAACGAGTGCTCCAGCCAGCCCTGCCGCCACGGGG
GCACCTGCCAGGACCGCGACAACGCCTACCTCTGCTTCTGCCTGAAGGGGACCACAGGACCCAACTGCGAG
ATCAACCTGGATGACTGTGCCAGCAGCCCCTGCGACTCGGGCACCTGTCTGGACAAGATCGATGGCTACGA
GTGTGCCTGTGAGCCGGGCTACACAGGGAGCATGTGTAACATCAACATCGATGAGTGTGCGGGCAACCCCT
GCCACAACGGGGGCACCTGCGAGGACGGCATCAATGGCTTCACCTGCCGCTGCCCCGAGCCCTACCACGAC
CCCACCTGCCTGTCTGAGGTCAATGAGTGCAACAGCAACCCCTGCGTCCACGGGGCCTGCCGGGACAGCCT
CAACGGGTACAAGTGCCACTGTGACCCTCGCTGGAGTGGGACCAACTGTGACATCAACAACAACGAGTGTG
AATCCAACCCTTGTGTCAACGGCGGCACCTGCAAAGACATGACCAGTGGCTACGTGTGCACCTGCCGGGAG
GGCTTCAGCGGTCCCAACTGCCAGACCAACATCAACGAGTGTGCGTCCAACCCATGTCTGAACAAGGGCAC
GTGTATTGACGACGTTGCCGGGTACAAGTGCAACTGCCTGCTGCCCTACACACGTGCCACGTGTGAGGTGG
TGCTGGCCCCGTGTGCCCCCAGCCCCTGCACAAACGGCGGGGAGTGCAGGCAATCCGAGGACTATGAGAGC
TTCTCCTGTGTCTGCCCCACGGCTGGGGCCAAAGGGCAGACCTGTGAGGTCGACATCAACGAGTGCGTTCT
GAGCCCGTCCCGGCACGGCGCATCCTGCCAGAACACCCACGGCGGCTACCGCTGCCACTGCCAGGCCGGCT
ACAGTGGGCGCAACTGCGAGACCGACATCGACGACTGCCGGCCCAACCCGTGTCACAACGGGGGCTCCTGC
ACAGACGGCATCAACACGGCCTTCTGCGACTGCCTGCCCGGCTTCCGGGGCACTTTCTGTGAGGAGGACAT
CAACGAGTGTGCCAGTGACCCCTGCCGCAACGGCGCCAACTGCACGGACTGCGTGGACAGCTACACGTGCA
CCTGCCCCGCAGGCTTCAGCGGGATCCACTGTGAGAACAACACGCCTGACTGCACAGAGAGCTCCTGCTTC
AACGGTGGCACCTCCGTGGACGGCATCAACTCGTTCACCTGCCTGTGTCCACCCGGCTTCACGGGCAGCTA
CTGCCAGCACGATCTCAATGAGTGCGACTCACAGCCCTGCCTGCATGGCGGCACCTGTCAGGACGGCTGCG
GCTCCTACAGGTGCACCTGCCCCCAGGGCTACACTGGCCCCAACTGCCAGAACCTTGTGCACTGGTGTGAC
TCCTCGCCCTGCAAGAACGGCGGCAAATGCTGCCACACCCACACCCAGTACCGCTGCGAGTGCCCCAGCGG
CTGGACCGGCCTTTACTGCGACGTGCCCAGCGTGTCCTGTGAGGTGGCTGCGCAGCGACAAGGTGTTGACG
TTGCCCGCCTGTGCCAGCATGGAGGGCTCTGTGTGGACGCGGGCAACACGCACCACTGCCGCTCCCAGGCG
GGCTACACAGGCAGCTACTGTGAGGACCTGGTGGACGAGTGCTCACCCAGCCCCTGCCAGAACGGGGCCAC
CTGCACGGACTACCTGGGCGGCTACTCCTGCAAGTGCGTGGCCGGCTACCACGGGGTGAACTGCTCTGAGG
AGATCGACGAGTGCCTCTCCCACCCCTGCCAGAACGGGGGCACCTGCCTCGACCTCCCCAACACCTACAAG
TGCTCCTGCCCACGCGGCACTCAGGGTGTGCACTGTGAGATCAACGTGGACGACTGCAATCCCCCCGTTGA
CCCCGTGTCCCGGAGCCCCAAGTGCTTTAACAACGGCACCTGCGTGGACCAGCTGGGCGGCTACAGCTGCA
CCTGCCCGCCGGGCTTCGTGGCTGAGCCCTGTGAGGGGGATGTCAACGAGTGCCTGTCCAATCCCTGCGAC
GCCCGTGGCACCCAGAACTGCGTGCAGCGCGTCAATGACTTCCACTGCGAGTGCCGTGCTGGTCACACCGG
GCGCCGCTGCGAGTCCGTCATCAATCGCTGCAAAGGCAAGCCCTGCAAGAATGGGGGCACCTGCGCCGTGG
CCTCCAACACCGCCCGCGGGTTCATCTGCAAGTGCCCTGCGGGCTTCGAGGGCGCCACGTGTGAGAATGAC
GCTCGTACCTGCGGCAGCCTGCGCTGCCTCAACGGCGCCACATGCATCTCCGGCCCGCGCAGCCCCACCTG
CCTCTGCCTGGGCCCCTTCACGGGCCCCGAATCCCAGTTCCCGGCCAGCAGCCCCTGCCTGGGCGGCAACC
CCTGCTACAACCAGGGGACCTGTGAGCCCACATCCGAGAGCCCCTTCTACCGTTGCCTGTGCCCCGCCAAA
TTCAACGGGCTCTTGTGCCACATCCTGGACTACAGCTTCGCGGGTGGCGCCGGGCGCGACATCCCCCCGCC
GCTGATCGAGGAGGCGTGCGAGCTGCCCGAGTGCCAGGAGGACGCGGGCAACAAGGTCTGCACCCTGCAGT
GCAACAACCACGCGTGCGGCTGGGACGGCGGTGACTGCTCCCTCAACTTCAATGACCCCTGCAAGAACTGC
ACGCAGTCTCTGCAGTGCTGGAAGTACTTCAGTGACGGCCACTGTGACAGCCAGTGCAACTCAGCCGGCTG
CCTCTTCGACGGCTTTGACTGCCAGCGTGCGGAAGGCCAGTGCAACCCCCTGTACGACCAGTACTGCAAGG
ACCACTTCAGCGACGGGCACTGCGACCAGGGCTGCAACAGCGCGGAGTGCGAGTGGGACGGGCTGGACTGT
GCGGAGCATGTACCCGAGAGGCTGGCGGCCGGCACGCTGGTGGTGGTGGTGCTGATGCCGCCGGAGCAGCT
GCGCAACAGCTCCTTCCACTTCCTGCGGAGCTCAGCCGCCTGCTGCACACCAACCATGGTCTTCAAGCGTG
ACGCACACGGCCAGCAGATGATCTTCCCCTACTACGGCCCCGAGGAGGAGCTGCGCAAGCACCCCATCAAG
CGTGCCGCCGAGGGCTGGGCCGCACCTGACGCCCTGCTCGGCCAGGTGAAGGCCTCGCTGCTCCCTGGTGG
CAGCGACGGTGGGCGGCGGCGGAGGGAGCTGGACCCCATGGACGTCCGCGGCTCCATCGTCTACCTGGAGA
TTGACAACCGGCAGTGTGTGCAGGCCTCCTCGCAGTGCTTCCAGAGTGCCACCGATGTGGCCGCATTCCTG
GGAGCGCTCGCCTCGCTGGGCAGCCTCAACATCCCCTACAAGATCGAGGCCGTGCAGAGTGAGACCGTGGA
GCCGCCCCCGCCGGCGCAGCTGCACTTCATGTACGTGGCGGCGGCCGCCTTTGTGCTTCTGTTCTTCGTGG
GCTGCGGGGTGCTGCTGTCCCGCAAGCGCCGGCGGCAGCATGGCCAGCTCTGGTTCCCTGAGGGCTTCAAA
GTGTCTGAGGCCAGCAAGAAGAAGCGGCGCGAGCCCCTCGGCGAGGACTCCGTGGGCCTCAAGCCCCTGAA
GAACGCTTCAGACCGTGCCCTCATGGACGACAACCAGAATGAGTGGGGGGACGAGGACCTGGAGACCAAGA
AGTTCCGGTTCGAGGAGCCCGTGGTTCTGCCTGACCTGGACGACCAGACAGACCACCGCCAGTGGACTCAG
CAGCACCTGGATCCCGCTGACCTGCGCATGTCTGCCATGCCCCCCACACCGCCCCAGGGTGAGGTTGACGC
CGACTGCATGGACGTCAATGTCCGCGGGCCTGATGGCTTCACCCCGCTCATGATCGCCTCCTGCAGCGGGG
GCGGCCTGGAGACGGGCAACAGCGAGGAAGAGGAGGACGCGCCGGCCGTCATCTCCGACTTCATCTACCAG
GGCGCCAGCCTGCACAACCAGACAGACCGCACGGGCGAGACCGCCTTGCACCTGGCCGCCCGCTACTCACG
CTCTGATGCCGCCAAGCGCCTGCTGGAGGCCAGCGCAGATGCCAACATCCAGGACAACATGGCCCGCACCC
CGCTGCATGCGGCTGTGTCTGCCGACGCACAAGGTGTCTTCCAGATCCTGATCCGGAACACGGCCACAGAC
CTGGATGCCCGCATGCATGATGGCACAACTCCACTGATCCTGGCTGCCCGCCTGGCCGTGGAGGGCATGCT
GGAGGACCTCATCAACTCACACGCCGACGTCAACGCCGTAGATGACCTGGGCAAGTCCGCCCTGCACTGGG
CCGCCGCCGTGAACAATGTGGATGCCGCAGTTGTGCTCCTGAAGAACGGGGCTAACAAAGATATGCAGAAC
AACAGGGAGGAGACACCCCTGTTTCTGGCCGCCCGGGAGCGCAGCTACGAGACCGCCAAGGTGCTGCTGGA
CCACTTTGCCAACCGGGACATCACGGATCATATGGACCGCCTGCCGCGCGACATCGCACAGGAGCGCATGC
ATCACGACATCGTGAGGCTGCTGGACGAGTACAACCTGGTGCGCAGCCCGCAGCTGCACGGAGCCCCGCTG
GGGGGCACGCCCACCCTGTCGCCCCCGCTCTGCTCGCCCAACGGCTACCTGGGCAGCCTCAACCCCGGCGT
GCAGGGCAAGAAGGTCCGCAAGCCCAGCAGCAAAGGCCTGGCCTGTGCAAGCAAGGAGGCCAAGGACCTCA
AGGCACGGAGCAAGAAGTCCCAGGATCGCAAGGGCTGCCTGCTGGACAGCTCCGGCATGCTCTCGCCCGTG
GACTCCCTGGAGTCACCCCATGGCTACCTGTCAGACGTGGCCTCGCCGCCACTGCTGCCCTCCCCGTTCCA
GCAGTCTCCGTCCGTGCCCCTCAACCACCTGCCTGGGATGCCCGACACCCACCTGGGCATCGGCCACCTGA
ACGTGGCGGCCAAGCCCGAGATGGCGGCGCTGGGTGGGGGCGGCCGGCTGGCCTTTCACACTGGCCCACCT
CGTCTCTCCCACCTGCCTGTGGCCTCTGGCACCAGCACCGTCCTGGGCTCCAGCAGCGGAGGGGCCCTGAA
TTTCACTGTGGGCGGGTCCACCAGTTTGAATGGTCAATGCGAGTGGCTGTCCCGGCTGCAGAGCGGCATGG
TGCCGAACCAATACAACCCTCTGCGGGGGAGTGTGGCACCAGGCCCCCTGAGCACACAGGCCCCCTCCCTG
CAGCATGGCATGGTAGGCCCGCTGCACAGTAGCCTTGCTGCCACCGCCCTGTCCCAGATGATGAGCTACCA
GGGCCTGCCCAGCACCCGGCTCGCCACCCAGCCTCACCTGGTGCAGACCCAGCAGGTGCAGCCACAAAACT
TACAGATGCAGCAGCAGAACCTGCAGCCAGCAAACATCCAGCAGCAGCAAAGCCTGCAGCCCCCACCACCA
CCACCACAGCCGCACCTTGGCGTGAGCTCAGCAGCCAGCGCCCACCTGGCCCGGAGCTTCCTCAGTGGAGA
GCCGAGCCAGGCAGACGTGCAGCCACTGGGCCCCAGCACCCTGGCGGTGCACACTATTCTGCCCCACGAGA
GCCCCGCCCTGCCCACGTCGCTGCCATCCTCGCTGGTCCCACCCGTGACCGCAGCCCAGTTCCTGACGCCC
CCCTCGCAGCACAGCTACTCCTCGCCTGTGGACAACACCCCCAGCCACCAGCTACAGGTGCCTGAGCACCC
CTTCCTGACCCCTTCGCCGGAGTCGCCCGACCAATGGTCCTCCTCGTCGCCGCACTCTAATGTGTCTGACT
GGTCTGAGGGCGTGTCGTCGCCCCCGACCTCCATGCAGTCCCACATCCCGCGCATCCCGGAGGCGTTCAAG
TAATAGCTCGAGGTGCCAGCAGCTC

The NOV33 nucleic acid has 7670 of 7693 bases (99%) identical to a gb:GENBANK-ID:AF308602|acc:AF308602.1 mRNA from Homo sapiens (NOTCH 1 (N1) mRNA, complete cds) (E=0.0).

A disclosed NOV33 polypeptide (SEQ ID NO:134) encoded by SEQ ID NO:133 is 2556 amino acid residues and is presented using the one-letter code in Table 33B. Signal P, Psort and/or Hydropathy results predict that NOV33 contains a signal peptide and is likely to be localized to the plasma membrane with a certainty of 0.4600. The most likely cleavage site for a NOV33 peptide is between amino acids 18 and 19: ALA-AR.

TABLE 33B
Encoded NOV33 protein sequence

(SEQ ID NO:134)

MPPLLAPLLCLALLPALAARGPRCSQPCETCLNGGKCEAANGTEACVCCCAFVGPRCQDPNPCLSTPCKNAG
TCHVVDRRGVADYACSCALGFSCPLCLTPLDNACLTNPCRNGGTCDLLTLTEYKCRCPPGWSGKSCQQADPC
ASNPCANGCQCLPFEASYICHCPPSFHGPTCRQDVNECGQKPGLCRHGGTCHNEVGSYRCVCRATHTGPNCE
RPYVPCSPSPCQNGGTCRPTGDVTHECACLPGFTGQNCEENIDDCPGNNCKNGGACVDGVNTYNCPCPPEWT
GQYCTEDVDECQLMPNACQNGGTCHNTHGGYNCVCVNGWTGEDCSENIDDCASAACFHGATCHDRVASFYCE
CPHCRTGLLCHLNDACISNPCNEGSNCDTNPVNGKATCTCPSCYTGPACSQDVDECSLGANPCEHAGKCINT
LGSFECQCLQGYTGPRCETDVNECVSNPCQNDATCLDQIGEFQCMCMPGYEGVHCEVNTDECASSPCLHNGR
CLDKINEFQCECPTGFTGHLCQYDVDECASTPCKNGAKCLDGPNTYTCVCTEGYTGTHCEVDIDECDPDPCH
YGSCKDGVATFTCLCRPGYTGHHCETNINECSSQPCRHCGTCQDRDNAYLCFCLKGTTGPNCEIMLDDCASS
PCDSGTCLDKIDGYECACEPGYTGSMCNINIDECAGNPCHNGGTCEDGINGFTCRCPEGYHDPTCLSEVNEC
NSNPCVHGACRDSLNGYKCDCDPGWSGTNCDINNNECESNPCVNGGTCKDMTSGYVCTCREGFSGPNCQTNI
NECASNPCLNKGTCIDDVAGYKCNCLLPYTGATCEVVLAPCAPSPCRNGGECRQSEDYESFSCVCPTAGAKG
QTCEVDINECVLSPCRHGASCQNTHGGYRCHCQAGYSGRNCETDIDDCRPNPCHNGGSCTDGINTAFCDCLP
GFRGTFCEEDINECASDPCRNGANCTDCVDSYTCTCPAGFSCIHCENNTPDCTESSCFNGGTCVDGINSFTC
LCPPGFTGSYCQHDVNECDSQPCLHGGTCQDGCGSYRCTCPQCYTGPNCQNLVHWCDSSPCKNGGKCWQTHT
QYRCECPSGWTGLYCDVPSVSCEVAAQRQGVDVARLCQHGGLCVDAGNTHHCRCQAGYTGSYCEDLVDECSP
SPCQNGATCTDYLGGYSCKCVAGYHGVNCSEEIDECLSHPCQNGGTCLDLPNTYKCSCPRGTQGVHCEINVD
DCNPPVDPVSRSPKCFNNGTCVDQVCGYSCTCPPGFVGERCEGDVNECLSNPCDARGTQNCVQRVNDFHCEC
RAGHTGRRCESVINGCKGKPCKNGGTCAVASNTARGFICKCPAGFEGATCENDARTCGSLRCLNGGTCISGP
RSPTCLCLGPFTGPECQFPASSPCLGGNPCYNQGTCEPTSESPFYRCLCPAKFNGLLCHILDYSFGGGAGRD
IPPPLIEEACELPECQEDAGNKVCSLQCNNHACGWDGGDCSLNFNDPWKNCTQSLQCWKYFSDGHCDSQCNS
AGCLFDGFDCQRAEGQCNPLYDQYCKDHFSDGHCDQGCNSAECEWDGLDCAEHVPERLAAGTLVVVVLMPPE
QLRNSSFHFLRELSRVLHTNVVFKRDAHGQQMIFPYYGREEELRKHPIKRAAEGWAAPDALLGQVKASLLPG
GSEGGRRRRELDPMDVRCSIVYLEIDNRQCVQASSQCFQSATDVAAFLGALASLGSLNIPYKIEAVQSETVE
PPPPAQLHFMYVAAAAFVLLFFVGCGVLLSRKRRRQHGQLWFPEGFKVSEASKKKRREPLCEDSVGLKPLKN
ASDGALMDDNQNEWGDEDLETKKFRFSEPVVLPDLDDQTDHRQWTQQHLDAADLRMSAMAPTPPQGEVDADC
MDVNVRGPDGFTPLMIASCSGGGLETGNSEEEEDAPAVISDFIYQGASLHNQTDRTGETALHLAARYSRSDA
AKRLLEASADANIQDNMGRTPLHAAVSADAQGVFQILIRNRATDLDARMEDSTTPLILAARLAVEGMLEDLI
NSHADVNAVDDLGKSALHWAAAVNNVDAAVVLLKNGANKDMQNNREETPLFLAAREGSYETAKVLLDHFANR
DITDHMDRLPRDIAQERMHHDIVRLLDEYNLVRSPQLHSAPLGGTPTLSPPLCSPNGYLGSLKPGVQGKKVR
KPSSKGLACGSKEAKDLKARRKKSQDGKGCLLDSSGMLSPVDSLESPHGYLSDVASPPLLPSPFQQSPSVPL
NHLPCMPDTHLGIGHLNVAAKPEMAALGGGGRLAFETGPPRLSHLPVASGTSTVLGSSSGGALNFTVGGSTS
LNGQCEWLSRLQSGMVPNQYNPLRGSVAPCPLSTQAPSLQHGMVGPLHSSLAASALSQMMSYQGLPSTRLAT
QPHLVQTQQVQPQNLQMQQQNLQPANIQQQQSLQPPPPPPQPHLGVSSAASGHLGRSFLSGEPSQADVQPLG
PSSLAVHTILPQESPALPTSLPSSLVPPVTAAQFLTPPSQHSYSSPVDNTPSHQLQVPEHPFLTPSPESPDQ
WSSSSPHSNVSDWSEGVSSPPTSMQSQIARIPEAFK

The disclosed NOV33 amino acid sequence has 2543 of 2556 amino acid residues (99%) identical to, and 2545 of 2556 amino acid residues (99%) similar to, the 2556 amino acid residue ptnr:TREMBLNEW-ACC:AAG33848 protein from Homo sapiens (Human) (Notch 1) (E=0.0).

NOV33 is predicted to be expressed in at least Adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea and uterus. This information was derived by determining the tissue sources of the sequences that were included in the invention including but not limited to SeqCalling sources, Public EST sources, Literature sources, and/or RACE sources.

In addition, NOV33 is predicted to be expressed in the following tissues because of the expression pattern of (GENBANK-ID: gb:GENBANK-ID:AF308602|acc:AF308602.1) a closely related Homo sapiens NOTCH 1 (N1) mRNA, complete cds homolog in species Homo sapiens: brain.

NOV33 also has homology to the amino acid sequences shown in the BLASTP data listed in Table 33C.

TABLE 33C
BLAST results for NOV33

| Gene Index/ | | Length | Identity | Positives | |
|---|---|---|---|---|---|
| Identifier | Protein/Organism | (aa) | (%) | (%) | Expect |
| gi\|11275980\|gb\|AAG33848.1\| | NOTCH 1 [Homo | 2556 | 2543/2556 | 2545/2556 | 0.0 |
| AF308602_1 (AF308602) | sapiens] | | (99%) | (99%) | |
| gi\|107215\|pir\|\|A40043 | notch protein | 2555 | 2537/2556 | 2541/2556 | 0.0 |
| | homolog TAN-1 | | (99%) | (99%) | |
| | precursor - human | | | | |
| gi\|1171777\|sp\|P46531\| | NEUROGENIC LOCUS | 2444 | 2429/2444 | 2431/2444 | 0.0 |
| NTC1_HUMAN | NOTCH PROTEIN | | (99%) | | |
| | HOMOLOG 1 | | | | |
| | PRECURSOR | | | | |
| | (TRANSLOCATION- | | | | |
| | ASSOCIATED NOTCH | | | | |
| | PROTEIN TAN-1) | | | | |
| gi\|6093542\|sp\|Q07008\| | NEUROGENIC LOCUS | 2531 | 2301/2557 | 2401/2557 | 0.0 |
| NTC1_RAT | NOTCH HOMOLOG | | (89%) | (92%) | |
| | PROTEIN 1 | | | | |
| | PRECURSOR | | | | |
| gi\|112074\|pir\|\|S18188 | notch protein | 2531 | 2300/2557 | 2399/2557 | 0.0 |
| | homolog - rat | | (89%) | (92%) | |

The homology of these sequences is shown graphically in the ClustalW analysis shown in Table 33D.

[image]

[image]

[image]

[image]

[image]

[image]

Tables 33E-I list the domain descriptions from DOMAIN analysis results against NOV33. This indicates that the NOV33 sequence has properties similar to those of other proteins known to contain this domain.

TABLE 33E
Domain Analysis of NOV33

gnl|Smart|smart00004, NL, Domain found in Notch and Lin-12;
The Notch protein is essential for the proper differenti-
ation of the Drosophila ectoderm. This protein contains 3 NL
domains. (SEQ ID NO:825)
CD-Length = 39 residues, 100.0% aligned
Score = 45.1 bits (105), Expect = 5e−05

| | | | |
|---|---|---|---|
| NOV33: | 1443 | PPLIEEACELPECQEDAGNKVCSLQCNNHACGWDGGDCS | 1481 |
| | | \| \|\| +\|+ \|+\|\| +\|\|\| \|\|\|\|\|\|\|\| | |
| Sbjct: | 1 | PQDPWSRCEDAQCWDKFGDGVCDEECNNAECLWDGGDCS | 39 |

TABLE 33F
Domain Analysis of NOV33

gnl|Smart|smart00004, NL, Domain found in Notch and Lin-12;
The Notch protein is essential for the proper differenti-
ation of the Drosophila ectoderm. This protein contains 3 NL
domains. (SEQ ID NO:825)
CD-Length = 39 residues, 94.9% aligned
Score = 44.7 bits (104), Expect = 7e−05

| | | | |
|---|---|---|---|
| NOV33: | 1486 | DPWKNCTQSLQCWKYFSDGHCDSQCNSAGCLFDGFDCQ | 1523 |
| | | \|\|\| \| \|\|\| \|\|\|\|\|+\|\|+\|\|\|+\|\|\|\| | |
| Sbjct: | 3 | DPWSRCE-DAQCWDKFGDGVCDEECNNAECLWDGGDCS | 39 |

TABLE 33G
Domain Analysis of NOV33

gnl|Smart|smart00004, NL, Domain found in Notch and Lin-12;
The Notch protein is essential for the proper differenti-
ation of the Drosophila ectoderm. This protein contains 3 NL
domains. (SEQ ID NO:825)
CD-Length = 39 residues, 97.4% aligned
Score = 41.2 bits (95), Expect = 7e−04

| | | | |
|---|---|---|---|
| NOV33: | 1522 | CQRAEGQCNPLYDQYCKDHFSDGHCDQGCNSAECEWDGLDC | 1562 |
| | | \| +\| \| \|\|\|\|\|\|\|+\|\|+\|\|\|\|\|\|\|\| | |
| Sbjct: | 1 | PQDPWSRCE---DAQCWDKFGDGVCDEECNNAECLWDOGDC | 38 |

TABLE 33H
Domain Analysis of NOV33

gnl|Pfam|pfam00023, ank, Ank repeat. Ankyrin
repeats generally consist of a beta, alpha, alpha,
beta order of secondary structures. The repeats
associate to form a higher order structure.
(SEQ ID NO:826)
CD-Length = 33 residues, 97.0% aligned
Score = 42.7 bits (99), Expect = 3e−04

| | | |
|---|---|---|
| NOV33: | 1929 | GETALHLAARYSRSDAAKRLLEASADANIQDN 1960 |
| | | \| \| \|\|\|\|\|\| + \| \|\|\|\| \|\| \| +\| |
| Sbjct: | 2 | GNTPLHLAARNGHLEVVKLLLEAGADVNARDK 33 |

TABLE 33I
Domain Analysis of NOV33

gnl|Pfam|pfam00066, notch, Notch (DSL) domain. The
Notch domain is also called the ‘DSL’ domain. The
notch proteins are transmembrane proteins with
extracellular domains of repeated ECF domains and
the notch (or DSL) domain N-terminal to that.
These proteins are generally involved in lateral
inhibition in developmental processes.
(SEQ ID NO: 826)
CD-Length = 38 residues, 81.6% aligned
Score = 42.0 bits (97), Expect = 4e−04

| | | |
|---|---|---|
| NOV33: | 1533 | YDQYCKDHFSDGHCDQGCNSAECEWDGLDCA 1563 |
| | | \| ++\| + \|++\| \|+\| \|\|+\| \| +\|\| \|\|+ |
| Sbjct: | 8 | YRRHCAERFANGVCNQECNNAACGFDGGDCS 38 |

Notch is a surface receptor. It transmits signals received from outside the cell to the cell's interior. Notch ligands, such as Delta, Serrate and Scabrous interact with epidermal growth factor repeats contained in Notch's extracellular domain. Notch plays an active role in the differentiation of glial cells and Notch influences the length and organisation of neuronal processes. Several homologs of the Drosophila Notch receptor and its ligands, Delta/Serrate, have been cloned in man. Three human disorders including a neoplasia (a T-cell acute lymphoblastic leukemia/lymphoma), a late onset neurological disease (CADASIL) and a developmental disorder (the Alagille syndrome) are associated with mutations in, respectively, the Notch 1, Notch3 and Jagged1 genes, pointing out the broad spectrum of Notch activity in humans (Joutel A, and Tournier-Lasserve E, 1998, Semin Cell Dev Biol, 9:619-25; Frisen J, and Lendahl U, 2001, Bioessays 23:3-7).

In Drosophila, the intracellular domain of Notch binds Suppressor of hairless, a multifunction transcription factor that acts as a signal transducing molecule shuttling between the cytoplasm and the nucleus. A nuclear function has been documented for the mammalian Notch homolog (Lu, 1996), as well as for Drosophila Notch (Struhl and Adachi, 1998, Cell 93:649-60). When Notch is bound by a ligand, a signal is passed across the cell membrane releasing the Suppressor of Hairless protein, freeing this protein to enter the nucleus and assume its role in activating transcription of enhancer of Split complex genes. E(spl)-C proteins act in turn to repress the adoption of neural and other differentiated states. Deltex, an intracellular docking protein, replaces Suppressor of Hairless as Su(H) leaves the site of interaction with the intracellular tail of Notch.

The Notch receptor's function is called neurogenic, but this confusing nomenclature refers to the phenotype established in the absence of functional Notch. Notch's function is to repress the adoption of differentiation by cells that carry the Notch protein. A look at the principle ligand of Notch (Delta) and its function makes the anti-neural function of Notch more easily understood. Delta is not secreted, but is cell bound. The Delta-Notch interaction serves a cell adhesive function between ligand and receptor bearing cells. The receptor bearing cell is inhibited in assuming a differentitated state, while the ligand bearing cell is free to do so. During neurogenesis, this latter cell delaminates, that is, it migrates out of the epithelial cell layer in which it formerly resided, and assumes the differentiated state of a neuroblast in its new physical location within the developing nervous system. Thus Notch is involved in neurogenesis with respect to cells that bears the ligands for Notch: Delta, Serrate and Scabrous.

Lateral inhibition is one of the major themes of development. The process of lateral inhibition and cell selection is repeated hundreds of times in Drosophila, with differentiation that takes place in nearly every kind of tissue. For example, Notch is required to limit the number of neuronal precursors, limit the number of muscle precursors, limit the growth of malphigian tubules, and regulate the growth of the ovary. Notch also functions as receptor for both Serrate and Delta in organizing the dorsal-ventral boundary of the wing. One important target of Serrate and Notch in this context is wingless (Diaz-Benjumea and Cohen, 1995, Development 121:4215-25). Two extreme models can be envisioned for lateral inhibition. The first implicates the Notch pathway in the choice of a single precursor via a negative feedback loop. This process could be random in some cases. The second model postulates that the precursor is pre-determined by some mechanism other than Notch signaling, and that Notch signaling then serves only to mediate mutual, uniform repression of other cells and ensure development of a single precursor. Studies concerning the physical spacing of precursors for the microchaetes of the peripheral nervous system suggest the existence of a regulatory loop under transcriptional control between Notch and its ligand Delta. Activation of Notch leads to repression of the achaete-scute genes, which are themselves known to regulate transcription of Delta; this regulation may perhaps be direct (Seugnet et al., 1997, Dev Biol. 192:585-98). Neuroblast segregation was studied in embryos lacking both the maternal and the zygotic forms of either Notch or Delta. A seven-up-LacZ marker was used to follow neuralization of 5-2 and 7-4 neuroblast groups. In the absence of Notch signaling, the cells with an equivalence group do not enter the neural differentiation pathway simultaneously. Neuralization within a group is progressive with two or three cells segregating early and several more later. This suggests that neural potential is not evenly distributed among these cells. A requirement for transcriptional regulation of Notch and/or Delta during neuroblast segregation in embryos was tested by providing Notch and Delta ubiquitously at uniform levels. Neuroblast segregation occurs normally under conditions of uniform Notch expression, suggesting that transcriptional regulation of Notch is not necessary for many aspects of development of the larval CNS and PNS. In particular, it is dispensable both before and after neuroblast segregation, implying that it is not a necessary component of neuroblast segregation, per se. Under conditions of uniform Delta expression, a single neuroblast segregates from each proneural group in 80% of the cases; in the remaining 20%, more than one neuroblast segregates from a single group of cells. Thus transcriptional regulation of Delta is largely dispensable, with only a small percentage of multiple neurons segregating in each cluster. Genes such as achaete, scute, extramacrochaete, and wingless could be responsible for local differences in proneural activity. Notch signaling would enable all cells to mutually repress one another; only a cell with an elevated neural potential could overcome this repression (Seugnet et al., 1997, Development 124:2015-25).

The development and patterning of the wing in Drosophila relies on a sequence of cell interactions molecularly driven by a number of ligands and receptors. Genetic analysis indicates that a receptor encoded by the Notch gene and a signal encoded by the wingless gene play a number of interdependent roles in this process and display very strong functional interactions. At certain times and places, during wing development, the expression of wingless requires Notch activity and that of its ligands Delta and Serrate. This has led to the proposal that all the interactions between Notch and wingless can be understood in terms of this regulatory relationship. This proposal has been tested by analyzing interactions between Delta- and Serrate-activated Notch signaling and Wingless signaling during wing development and patterning. Cell death caused by expressing dominant negative Notch molecules during wing development cannot be rescued by coexpressing Nintra. This suggests that the dominant negative Notch molecules cannot only disrupt Delta and Serrate signaling but can also disrupt signaling through another pathway. One possibility is the Wingless signaling pathway, since the cell death caused by expressing dominant negative Notch molecules can be rescued by activating Wingless signaling. Furthermore, the outcome of the interactions between Notch and Wingless signaling differs when Wingless signaling is activated by expressing either Wingless itself or an activated form of the Armadillo. For example, the effect of expressing the activated form of Armadillo with a dominant negative Notch on the patterning of sense organ precursors in the wing resembles the effects of expressing Wingless alone. This result suggests that signaling activated by Wingless leads to two effects: a reduction of Notch signaling and an activation of Armadillo (Brennan, 1999, Curr Biol, 9:707-10).

Expression of a dominant negative Notch molecule (Extracellular Notch or ECN) throughout the developing wing mimics the effects of loss of Notch function. However, Nintra cannot rescue the cell death caused by overexpressing ECN. Since Nintra provides constitutive signaling for Delta and Serrate during wing development and the effects of ECN are mediated by the sequestration of extracellular molecules that can interact with Notch, this suggests that the ECN molecule is sequestering extracellular molecules other than Delta and Serrate and attenuating signaling through another pathway. One candidate pathway is the Wingless signaling pathway, since the cell death caused by expressing the ECN can be rescued by activating Wingless signaling. Therefore, it is possible that the ECN molecule is sequestering the Wingless protein. The possibility that Wingless can bind the extracellular domain of Notch is supported by the following results, in particular, by two observations: first, that some of the deleterious effects of ECN can be suppressed by Wingless, but not Wingless signaling in the form of a constitutively active Armadillo molecule; and second, that this interaction requires specific EGF-like repeats of Notch, namely repeats 17-19 and 24-26 but not 10-12. Evidence for a physical interaction between Notch and Wingless has also been provided recently by Wesley (1999, Mol Cell Biol. 19:5743-58) who finds that the Wingless protein is enriched in a biopanning assay designed to identify proteins that interact with the extracellular domain of the Notch protein and that Wingless can be immunoprecipitated with Notch from embryo extracts and cultured cells. These experiments also show that the association of Wingless with Notch requires the integrity of a region of Notch centered around EGF-like repeats 24-26 (Wesley, 1999, Mol Cell Biol. 19:5743-58) which these experiments indicate are essential for the interactions that are described between Wingless and ECN during wing development and patterning (Brennan et al., 1999, Curr Biol. 9:707-10). The interaction of Wingless and Notch signaling that has been observed might also be important during normal neural development. Wingless and Delta have opposite effects during neurogenesis; Wingless promotes while Delta suppresses the development of sense organs. Various experiments suggest that during the segregation of neural precursors a reduction of Notch signaling in the precursors themselves is as important as the Delta-mediated activation of Notch signaling in the surrounding cells. It is possible that, like the activation of Notch by Delta, the suppression of Notch signaling is an active process mediated by the interaction of Wingless and Dishevelled with Notch. If this were the case, since both Delta and Wingless have spatially and temporally regulated patterns of gene expression, their interactions with Notch could contribute to the well-documented bias in the appearance of precursors from clusters of cells with neural potential. This competitive interaction could also account for the observed increases in Wingless signaling associated with reductions in Notch signaling during lateral inhibition (Brennan et al., 1999, Curr Biol. 9:707-10; Brennan et al, 1999, Dev Biol. 216:210-29).

The NOV33 nucleic acid of the invention encoding a Notch1-like protein includes the nucleic acid whose sequence is provided in Table 33A, or a fragment thereof. The invention also includes a mutant or variant nucleic acid any of whose bases may be changed from the corresponding base shown in Table 33A while still encoding a protein that maintains its Notch1-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 1% of the residues may be so changed.

The NOV33 protein of the invention includes the Notch1-like protein whose sequence is provided in Table 33B. The invention also includes a mutant or variant protein any of whose residues may be changed from the corresponding residue shown in Table 33B while still encoding a protein that maintains its Notch1-like activities and physiological functions, or a functional fragment thereof. In the mutant or variant protein, up to about 11% of the bases may be so changed.

The NOV33 nucleic acids and proteins of the invention are useful in potential diagnostic and therapeutic applications implicated in various diseases and disorders, described below and/or other pathologies. For example, the compositions of the present invention will have efficacy for treatment of patients suffering from: neoplasia such as T-cell acute lymphoblastic leukemia/lymphoma and mammary carcinomas, a late onset neurological disease like CADASIL and a developmental disorder such as the Alagille syndrome, familial and congenital cholestatic diseases, Hereditary vascular dementia, neurological diseases and other diseases, disorders and conditions of the like.

NOV33 nucleic acids and polypeptides are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. For example the disclosed NOV33 protein have multiple hydrophilic regions, each of which can be used as an immunogen. This novel protein also has value in development of powerful assay system for functional analysis of various human disorders, which will help in understanding of pathology of the disease and development of new drug targets for various disorders.

NOV34

A disclosed NOV34 nucleic acid of 935 nucleotides (also referred to as CG56715-01) encoding a novel Olfactory Receptor-like protein is shown in Table 34A. An open reading frame was identified beginning with an ACA codon, which codes for the amino acid Threonine, at nucleotides 2-4 and ending with a TGA codon at nucleotides 932-934. Putative untranslated regions, if any, found upstream from the initiation codon and downstream from the termination codon are underlined in Table 34A, and the start and stop codons are in bold letters.

TABLE 34A
NOV34 Nucleotide Sequence

(SEQ ID NO:135)
CACACAAGAAGGCATCTACTTCATCCTCACGGACATCCCTGGATTTGAGGCCTCCCACATCTGGATCTCCA
TCCCCGTCTGCTGTCTCTACACCATCTCCATCATGGCCAATACCACCATCCTCACTGTCATTCGCACAGAG
CCATCTGTCCACCAGCGCATGTATCTGTTTCTCTCCATGCTGGCCCTGACGGACCTGGGTCTCACCCTCAC
CACCCTACCCACAGTCATGCAGCTTCTCTGGTTCAACGTTCGTAGAATCAGCTCTGAGGCCCGTTTTGCTC
AGTTTTTCTTCCTTCATGGATTCTCCTTTATGGAGTCTTCTGTCCTCCTGGCTATGTCCGTTGACTGCTAT
GTGGCCATCTGCTGTCCCCTCCATTATGCCTCCATCCTCACCAATGAAGTCATTGGTAGAACTGGGTTAGC
CATCATTTGCTGCTGTGTTCTGGCGGTTCTTCCCTCCCTTTTCTTACTCAAGCGACTGCCTTTCTGCCACT
CCCACCTTCTCTCTCGCTCCTATTGCCTCCACCAGGATATGATCCGCCTGGTCTGTGCTGACATCAGGCTC
AACAGCTGGTATGGATTTGCTCTTGCCTTGTTCATTATTATCGTGGATCCTCTGCTCATTGTGATCTCCTA
TACACTTATTCTGAAAAATATCTTGGGCACAGCCACCTGGGCTGAGCGACTCCGTGCCCTCAATAACTGCC
TGTCCCACATTCTGGCTGTCCTGGTCCTCTACATTCCCATGGTTGGTGTATCTATGACTCATCGCTTTGCC
AAGCATGCCTCTCCACTGGTCCATGTTATCATGGCCAATATCTACCTGCTGGCACCCCCGGTGATGAACCC
CATCATTTACAGTGTAAAGAACAAGCAGATCCAATGGGGAATGTTAAATTTCCTTTCCCTCAAAAATATGC
ATTCAAGATGAG

The NOV34 nucleic acid has 578 of 903 bases (64%) identical to a gb:GENBANK-ID:AF137396|acc:AF137396.2 mRNA from Homo sapiens (ubiquilin 3, HOR 5′Beta14, HOR5′Beta13, HOR5′Beta12, and HOR5′Beta11 genes, complete cds; HOR 5′Beta10 and HOR5′Beta9 pseudogenes, complete sequence; HOR5′Beta8 and HOR5′Beta7 genes, complete cds; CHR11ORF1 and amphiphysin pseudogenes, complete sequence; HOR5′Beta6 and HOR5′Beta5 genes, complete cds; HOR5′Beta4 pseudogene, complete sequence; HOR 5′Beta3 genes, complete cds; HOR5′Beta2 pseudogene, complete sequence; and HOR 5′Beta1 gene, complete cds) (E=6.1e⁻⁵⁰).

A disclosed NOV34 polypeptide (SEQ ID NO:136) encoded by SEQ ID NO:135 is 310 amino acid residues and is presented using the one-letter code in Table 34B. Signal P, Psort and/or Hydropathy results predict that NOV34 contains a signal peptide and is likely to be localized to the plasma membrane with a certainty of 0.6000. The most likely cleavage site for a NOV34 peptide is between amino acids 36 and 37: IMG-NT.

TABLE 34B
Encoded NOV34 protein sequence

(SEQ ID NO:136)

TQEGIYFILTDIPGFEASHIWISIPVCCLYTISIMGNTTILTVIRTEPSVHQRMYLFLSMLALTDLGLTLTT
LPTVMQLLWFNVRRISSEARFAQFFFLHGFSFMESSVLLAMSVDCYVAICCPLHYASILTNEVIGRTGLAII
CCCVLAVLPSLFLLKRLPFCHSHLLSRSYCLHQDMIRLVCADIRLNSWYGFALALFIIIVDPLLIVISYTLI
LKNILGTATWAERLRALNNCLSHILAVLVLYIPMVGVSMTHRFAKHASPLVHVIMANIYLLAPPVMNPIIYS
VKNKQIQWGMLNFLSLKNMHSR

The disclosed NOV34 amino acid sequence has 160 of 296 amino acid residues (54%) identical to, and 210 of 296 amino acid residues (70%) similar to, the 319 amino acid residue ptnr:TREMBLNEW-ACC:AAG41684 protein from Mus musculus (Mouse) (MOR 3′BETA4) (E=6.3e⁻⁸³).

NOV34 is predicted to be expressed in at least Apical microvilli of the retinal pigment epithelium, arterial (aortic), basal forebrain, brain, Burkitt lymphoma cell lines, corpus callosum, cardiac (atria and ventricle), caudate nucleus, CNS and peripheral tissue, cerebellum, cerebral cortex, colon, cortical neurogenic cells, endothelial (coronary artery and umbilical vein) cells, palate epithelia, eye, neonatal eye, frontal cortex, fetal hematopoietic cells, heart, hippocampus, hypothalamus, leukocytes, liver, fetal liver, lung, lung lymphoma cell lines, fetal lymphoid tissue, adult lymphoid tissue, those that express MHC II and III nervous, medulla, subthalamic nucleus, ovary, pancreas, pituitary, placenta, pons, prostate, putamen, serum, skeletal muscle, small intestine, smooth muscle (coronary artery in aortic) spinal cord, spleen, stomach, taste receptor cells of the tongue, testis, thalamus and thymus tissue. This information was derived by determining the tissue sources of the sequences that were included in the invention including but not limited to SeqCalling sources, Public EST sources, Literature sources, and/or RACE sources.

NOV34 also has homology to the amino acid sequences shown in the BLASTP data listed in Table 34C.

TABLE 34C
BLAST results for NOV34

| Gene Index/ | | Length | Identity | Positives | |
|---|---|---|---|---|---|
| Identifier | Protein/Organism | (aa) | (%) | (%) | Expect |
| gi\|17472775\|ref\|XP_{—} | similar to MOR | 317 | 298/300 | 298/300 | e−136 |
| 061808.1\| | 3Beta4 (H. | | (99%) | (99%) | |
| (XM_061808) | sapiens) [Homo | | | | |
| | sapiens] | | | | |
| gi\|17456767\|ref\|XP_{—} | similar to | 879 | 168/289 | 215/289 | 6e−81 |
| 061618.1\| | prostate specific | | (58%) | (74%) | |
| (XM_061618) | G-protein coupled | | | | |
| | receptor (H. | | | | |
| | sapiens) [Homo | | | | |
| | sapiens] | | | | |
| gi\|17456753\|ref\|XP_{—} | similar to MOR | 315 | 154/294 | 214/294 | 5e−75 |
| 061614.1\| | 3Beta4 (H. | | (52%) | (72%) | |
| (XM_061614) | sapiens) [Homo | | | | |
| | sapiens] | | | | |
| gi\|17472781\|ref\|XP_{—} | similar to | 312 | 150/290 | 204/290 | 6e−74 |
| 061811.1\| | OLFACTORY | | (51%) | (69%) | |
| (XM_061811) | RECEPTOR 51I2 | | | | |
| | (HOR5BETA12) (H. | | | | |
| | sapiens) [Homo | | | | |
| | sapiens] | | | | |
| gi\|11908220\|gb\|AAG41684.1\| | MOR 3′ Beta4 [Mus | 319 | 159/294 | 209/294 | 2e−72 |
| (AF133300) | musculus] | | (54%) | (71%) | |

The homology of these sequences is shown graphically in the ClustalW analysis shown in Table 34D.

[image]

[image]

[image]

Table 34E list the domain descriptions from DOMAIN analysis results against NOV34. This indicates that the NOV34 sequence has properties similar to those of other proteins known to contain this domain.

TABLE 34E
Domain Analysis of NOV34

gnl|Pfam|pfam00001, 7tm_1, 7 transoembrane receptor (rhodopsin
family). (SEQ ID NO:810)
CD-Length = 254 residues, 100.0% aligned
Score = 41.6 bits (96), Expect = 7e−05

| | | | |
|---|---|---|---|
| NOV40: | 36 | GNTTILTVIRTEPSVHQRMYLFLSMLALTDLGLTLTTLPTVMQLLWFNVRRISSEARFAQ | 95 |
| | | \|\| ++ \|\| + +\|\| \|\|+ \|\| \|\| \| + \| | |
| Sbjct: | 1 | GNLLVILVILRTKKLRTPTNIFLLNLAVADLLFLLTLPPWALYYLVGGDWVFGDALCKLV | 60 |
| NOV40: | 96 | FFFLHGFSFMESSVLLAMSVDCYVAICCPLHYASILTNEVIGRTGLAIICCCVLAVLPSL | 155 |
| | | + +\| \|+\|+\| \|+\|\| \|\| \| \| \| \| + \|+ \|\|\|+\| \| | |
| Sbjct: | 61 | GALFVVNGYASILLLTAISIDRYLAIVHPLRYRRIRT---PRRAKVLILLVWVLALLLS- | 116 |
| NOV40: | 156 | FLLKRLPFCHSHLLSRSYCLHQDMIRLVCADIRLNSWYGFALALFIIIVDPLLIVISYTL | 215 |
| | | \|\| \| + + + + + \| \| ++ \|+\|++ \|\| | |
| Sbjct: | 117 | -----LPPLLFSWLRTVEEGNTTVCLIDFPEESVKRSYVLLSTLVGFVLPLLVILVCYTR | 171 |
| NOV40: | 216 | IL--------KNILGTATWAERLRALNNCLSHILAVLVLYIPMVGVSMTHRFAKHASPLV | 267 |
| | | \|\| + +\| \| ++ ++ ++\| \| + + \| | |
| Sbjct: | 172 | ILRTLRKRARSQRSLKRRSSSERKAAKMLLVVVVVFVLCWLPYHIVLLLDSLCLLSIWRV | 231 |
| NOV40: | 268 | HVIMANIYLL---APPVMNPIIY 287 | |
| | | \| \| +\|\|\|\|\| | |
| Sbjct: | 232 | LPTALLITLWLAYVNSCLNPIIY 254 | |

G-Protein Coupled Receptor (GPCRs) have been identified as an extremely large family of protein receptors in a number of species. At the phylogenetic level they can be classified into four major subfamilies. These receptors share a seven transmembrane domain structure with many neurotransmitter and hormone receptors. They are likely to be involved in the recognition and transduction of various signals mediated by G-Proteins, hence their name G-Protein Coupled Receptors. The human GPCR genes are generally intron-less and belong to four gene subfamilies, displaying great sequence variability. These genes are dominantly expressed in olfactory epithelium.

Olfactory receptors (ORs) have been identified as extremely large family of GPCRs in a number of species. As members of the GPCR family, these receptors share a seven transmembrane domain structure with many neurotransmitter and hormone receptors, and are likely to underlie the recognition and G-protein-mediated transduction of odorant signals. Like GPCRs, the ORs they can be expressed in a variety of tissues where they are thought to be involved in recognition and transmission of a variety of signals. The human OR genes are typically intron-less and belong to four different gene subfamilies, displaying great sequence variability. These genes are dominantly expressed in olfactory epithelium.

The NOV34 nucleic acid of the invention encoding a Olfactory Receptor-like protein includes the nucleic acid whose sequence is provided in Table 34A, or a fragment thereof. The invention also includes a mutant or variant nucleic acid any of whose bases may be changed from the corresponding base shown in Table 34A while still encoding a protein that maintains its Olfactory Receptor-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 36% of the residues may be so changed.

The NOV34 protein of the invention includes the Olfactory Receptor-like protein whose sequence is provided in Table 34B. The invention also includes a mutant or variant protein any of whose residues may be changed from the corresponding residue shown in Table 34B while still encoding a protein that maintains its Olfactory Receptor-like activities and physiological functions, or a functional fragment thereof. In the mutant or variant protein, up to about 49% of the bases may be so changed.

The NOV34 nucleic acids and proteins of the invention are useful in potential diagnostic and therapeutic applications implicated in various diseases and disorders described below and/or other pathologies. For example, the compositions of the present invention will have efficacy for treatment of patients suffering from: developmental diseases, MHCII and III diseases (immune diseases), Taste and scent detectability Disorders, Burkitt's lymphoma, Corticoneurogenic disease, Signal Transduction pathway disorders, Retinal diseases including those involving photoreception, Cell Growth rate disorders, Cell Shape disorders, Feeding disorders, control of feeding, potential obesity due to over-eating, potential disorders due to starvation (lack of apetite), noninsulin-dependent diabetes mellitus (NIDDM1), bacterial, fungal, protozoal and viral infections (particularly infections caused by HIV-1 or HIV-2), pain, cancer (including but not limited to Neoplasm, adenocarcinoma, lymphoma, prostate cancer, uterus cancer), anorexia, bulimia, asthma, Parkinson's disease, acute heart failure, hypotension, hypertension, urinary retention, osteoporosis, Crohn's disease, multiple sclerosis, treatment of Albright Hereditary Ostocodystrophy, angina pectoris, myocardial infarction, ulcers, asthma, allergies, benign prostatic hypertrophy, psychotic and neurological disorders, including anxiety, schizophrenia, manic depression, delirium, dementia, severe mental retardation, Dentatorubro-pallidoluysian atrophy (DRPLA), Hypophosphatemic rickets, autosomal dominant (2) Acrocallosal syndrome and dyskinesias, such as Huntington's disease or Gilles de la Tourette syndrome and other diseases, disorders and conditions of the like.

NOV34 nucleic acids and polypeptides are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. For example the disclosed NOV34 protein have multiple hydrophilic regions, each of which can be used as an immunogen. This novel protein also has value in development of powerful assay system for functional analysis of various human disorders, which will help in understanding of pathology of the disease and development of new drug targets for various disorders.

NOV35

A disclosed NOV35 nucleic acid of 1102 nucleotides (also referred to as CG56718-01) encoding a novel Olfactory Receptor-like protein is shown in Table 35A. An open reading frame was identified beginning with an ATG initiation codon at nucleotides 92-94 and ending with a TGA codon at nucleotides 1049-1051. Putative untranslated regions, if any, found upstream from the initiation codon and downstream from the termination codon are underlined in Table 35A, and the start and stop codons are in bold letters.

TABLE 35A
NOV35 Nucleotide Sequence

(SEQ ID NO:137)

xATAACTCTACTAACTAAAAGAAAATGTATCTAAATATTTTACATAACACTTAATGTATCACTTCTATAGGA
TGGAAGCCTTTTGAGGACAAATGCTGCAAAACCAGGACACCATGGAAATCCTAAGCAACTCAACATCTAAA
TTTCCAACCTTCTTGTTGACCGGCATTCCTGGCCTAGAGTCTGCCCATGTCTGGATCTCCATTCCTTTCTG
TTGTTTTTATGCCATTGCCCTCTCTGGGAACAGCGTGATCCTGTTTGTCATCATTACCCAGCAGAGTCTCC
ATGAACCCATGTATTATTTCCTCTTCAGGCTATCAGCCACTGATCTGGGCTTGACTGTTTCTTCATTGTCA
ACAACATTAGGTATCCTCTGGTTTGAGGCACGTGAAATCAGTCTATATAGCTGCATTGTCCAGATGTTTTT
TCTTCATGGATTCACTTTTATGGAATCTGGAGTGCTGGTGGCTACAGCCTTTGACCGTTATGTGGCCATCT
GTGACCCTCTGAGGCACACTACCATTCTCACTAATTCCAGAATCATTCAAATGGGTCTTCTGATGATTACA
CGTGCTATAGTACTAATATTGCCACTACTTTTGCTCCTTAAGCCTCTCTATTTCTGTAGAATGAATGCCCT
TTCTCACTCCTATTGTTACCATCCAGATGTGATTCAATTAGCATGTTCAGACATTCGGGCAAATAGCATCT
GTGGATTAATTGATCTCATCCTGACCACTGGAATAGATACACCATGCATTGTCCTGTCATATATCTTAATT
ATTCACTCTGTCCTCAGAATTGCCTCCCCTGAAGAATGGCACAAGGTCTTCAGCACCTGTGTCTCCCATGT
GGGAGCAGTTGCTTTCTTCTACATCCACATGCTGAGCCTGTCCTTGGTGTATCGCTATGGTCGGTCAGCCC
CCAGAGTAGTCCATTCAGTGATGGCTAATGTATACCTGCTTTTACCCCCTGTGCTCAACCCCATCATCGAC
AGTGTAAAAACAAAACAAATCCGCAAGGCTATGCTCAGTCTGCTGCTTACAAAATGAACAGACATAGTTTT
ATTTGATACAAACCTGGCATGAATGACTTGCACTGTA

The NOV35 nucleic acid, located on chromosome 11, has 636 of 1006 bases (63%) identical to a gb:GENBANK-ID:AF133300|acc:AF133300.2 mRNA from Mus musculus (MOR 3′Beta1, MOR 3′Beta2, MOR 3′Beta3, and MOR 3′Beta4 genes, complete cds; Cbx3 pseudogene, complete sequence; and MOR 3′Beta5 and MOR 3′Beta6 genes, complete cds) (E=1.2e⁻⁵¹).

A disclosed NOV35 polypeptide (SEQ ID NO:138) encoded by SEQ ID NO:137 is 319 amino acid residues and is presented using the one-letter code in Table 35B. Signal P, Psort and/or Hydropathy results predict that NOV35 contains a signal peptide and is likely to be localized to the plasma membrane with a certainty of 0.6000.

TABLE 35B
Encoded NOV35 protein sequence

(SEQ ID NO:138)

MLQNQDTMEILSNSTSKFPTFLLTGIPGLESAHVWISIPFCCFYAIALSGNSVILFVIITQQSLHEPMYYFL
FRLSATDLGLTVSSLSTTLGILWFEAREISLYSCIVQMFFLHGFTFMESGVLVATAFDRYVAICDPLRHTTI
LTNSRIIQMGLLMITRAIVLILPLLLLLKPLYFCRMNALSHSYCYHPDVIQLACSDIRANSICGLIDLILTT
GIDTPCIVLSYILIIHSVLRIASPEEWHKVFSTCVSHVGAVAFFYIHMLSLSLVYRYGRSAPRVVHSVMANV
YLLLPPVLNPIIDSVKTKQIRKAMLSLLLTK

The disclosed NOV35 amino acid sequence has 160 of 309 amino acid residues (51%) identical to, and 219 of 309 amino acid residues (70%) similar to, the 321 amino acid residue ptnr:TREMBLNEW-ACC:AAG42364 protein from Homo sapiens (Human) (odorant receptor HOR3′BETA1) (E=2.5e⁻⁸¹).

NOV35 is predicted to be expressed in at least Apical microvilli of the retinal pigment epithelium, arterial (aortic), basal forebrain, brain, Burkitt lymphoma cell lines, corpus callosum, cardiac (atria and ventricle), caudate nucleus, CNS and peripheral tissue, cerebellum, cerebral cortex, colon, cortical neurogenic cells, endothelial (coronary artery and umbilical vein) cells, palate epithelia, eye, neonatal eye, frontal cortex, fetal hematopoietic cells, heart, hippocampus, hypothalamus, leukocytes, liver, fetal liver, lung, lung lymphoma cell lines, fetal lymphoid tissue, adult lymphoid tissue, Those that express MHC II and III nervous, medulla, subthalamic nucleus, ovary, pancreas, pituitary, placenta, pons, prostate, putamen, serum, skeletal muscle, small intestine, smooth muscle (coronary artery in aortic) spinal cord, spleen, stomach, taste receptor cells of the tongue, testis, thalamus and thymus tissue. This information was derived by determining the tissue sources of the sequences that were included in the invention including but not limited to SeqCalling sources, Public EST sources, Literature sources, and/or RACE sources.

NOV35 also has homology to the amino acid sequences shown in the BLASTP data listed in Table 35C.

TABLE 35C
BLAST results for NOV35

| Gene Index/ | | Length | Identity | Positives | |
|---|---|---|---|---|---|
| Identifier | Protein/Organism | (aa) | (%) | (%) | Expect |
| gi\|17456801\|ref\|XP_{—} | similar to | 342 | 196/312 | 251/312 | 1e−95 |
| 061626.1\| | OLFACTORY | | (62%) | (79%) | |
| (XM_061626) | RECEPTOR 51I2 | | | | |
| | (HOR5BETA12) (H. | | | | |
| | sapiens) [Homo | | | | |
| | sapiens] | | | | |
| gi\|17456777\|ref\|XP_{—} | similar to | 327 | 165/312 | 218/312 | 6e−77 |
| 061621.1\| | olfactory | | (52%) | (68%) | |
| (XM_061621) | receptor-like | | | | |
| | protein COR3beta | | | | |
| | (H. sapiens) | | | | |
| | [Homo sapiens] | | | | |
| gi\|17456767\|ref\|XP_{—} | similar to | 879 | 158/304 | 218/304 | 8e−74 |
| 061618.1\| | prostate specific | | (51%) | (70%) | |
| (XM_061618) | G-protein coupled | | | | |
| | receptor (H. | | | | |
| | sapiens) [Homo | | | | |
| | sapiens] | | | | |
| gi\|17472781\|ref\|XP_{—} | similar to | 312 | 155/295 | 209/295 | 1e−73 |
| 061811.1\| | OLFACTORY | | (52%) | (70%) | |
| (XM_061811) | RECEPTOR 51I2 | | | | |
| | (HOR5BETA12) (H. | | | | |
| | sapiens) [Homo | | | | |
| | sapiens] | | | | |
| gi\|18202242\|sp\|088628\| | Olfactory | 320 | 147/305 | 206/305 | 4e−73 |
| OXE2_RAT | receptor 51E2 (G- | | (48%) | (67%) | |
| | protein coupled | | | | |
| | receptor RA1c) | | | | |

The homology of these sequences is shown graphically in the ClustalW analysis shown in Table 35D.

[image]

[image]

[image]

Table 35E list the domain descriptions from DOMAIN analysis results against NOV35. This indicates that the NOV35 sequence has properties similar to those of other proteins known to contain this domain.

TABLE 35E
Domain Analysis of NOV35

gnl|Pfam|pfam00001, 7tm_1, 7 transmembrane receptor (rhodopsin family)
(SEQ ID NO:810)
CD-Length = 254 residues, 40.9% aligned
Score = 56.2 bits (134), Expect = 3e−09

| | | | |
|---|---|---|---|
| NOV35: | 50 | GNSVILFVIITQQSLHEPMYYFLFRLSATDLGLTVSSLSTTLGILWFEAREISLYSCIVQ | 109 |
| | | \|\| +++ \|\|+ + \| \| \|\| \|+ \|\| ++ \| \| \| + | |
| Sbjct: | 1 | GNLLVILVILRTKKLRTPTNIFLLNLAVADLLFLLTLPPWALYYLVGGDWVFGDALCKLV | 60 |
| NOV35: | 110 | MFFLHGFTFMESGVLVATAFDRYVAICDPLRHTTILTNSRIIQM 153 | |
| | | + +\| \| + \|\|\|+\|\| \|\|\|+ \| \| \| + | |
| Sbjct: | 61 | GALFVVNGYASILLLTAISIDRYLAIVHPLRYRRIRTPRRAKVL 104 | |

G-Protein Coupled Receptor (GPCRs) have been identified as an extremely large family of protein receptors in a number of species. At the phylogenetic level they can be classified into four major subfamilies. These receptors share a seven transmembrane domain structure with many neurotransmitter and hormone receptors. They are likely to be involved in the recognition and transduction of various signals mediated by G-Proteins, hence their name G-Protein Coupled Receptors. The human GPCR genes are generally intron-less and belong to four gene subfamilies, displaying great sequence variability. These genes are dominantly expressed in olfactory epithelium.

Olfactory receptors (ORs) have been identified as extremely large family of GPCRs in a number of species. As members of the GPCR family, these receptors share a seven transmembrane domain structure with many neurotransmitter and hormone receptors, and are likely to underlie the recognition and G-protein-mediated transduction of odorant signals. Like GPCRs, the ORs they can be expressed in a variety of tissues where they are thought to be involved in recognition and transmission of a variety of signals. The human OR genes are typically intron-less and belong to four different gene subfamilies, displaying great sequence variability. These genes are dominantly expressed in olfactory epithelium.

The NOV35 nucleic acid of the invention encoding a Olfactory Receptor-like protein includes the nucleic acid whose sequence is provided in Table 35A, or a fragment thereof. The invention also includes a mutant or variant nucleic acid any of whose bases may be changed from the corresponding base shown in Table 35A while still encoding a protein that maintains its Olfactory Receptor-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 37% of the residues may be so changed.

The NOV35 protein of the invention includes the Olfactory Receptor-like protein whose sequence is provided in Table 35B. The invention also includes a mutant or variant protein any of whose residues may be changed from the corresponding residue shown in Table 35B while still encoding a protein that maintains its Olfactory Receptor-like activities and physiological functions, or a functional fragment thereof. In the mutant or variant protein, up to about 52% of the bases may be so changed.

The NOV35 nucleic acids and proteins of the invention are useful in potential diagnostic and therapeutic applications implicated in various diseases and disorders described below and/or other pathologies. For example, the compositions of the present invention will have efficacy for treatment of patients suffering from: developmental diseases, MHCII and III diseases (immune diseases), Taste and scent detectability Disorders, Burkitt's lymphoma, Corticoneurogenic disease, Signal Transduction pathway disorders, Retinal diseases including those involving photoreception, Cell Growth rate disorders, Cell Shape disorders, Feeding disorders, control of feeding, potential obesity due to over-eating, potential disorders due to starvation (lack of apetite), non insulin-dependent diabetes mellitus (NIDDM1), bacterial, fungal, protozoal and viral infections (particularly infections caused by HIV-1 or HIV-2), pain, cancer (including but not limited to Neoplasm, adenocarcinoma, lymphoma, prostate cancer, uterus cancer), anorexia, bulimia, asthma, Parkinson's disease, acute heart failure, hypotension, hypertension, urinary retention, osteoporosis, Crohn's disease, multiple sclerosis, treatment of Albright Hereditary Ostocodystrophy, angina pectoris, myocardial infarction, ulcers, asthma, allergies, benign prostatic hypertrophy, psychotic and neurological disorders, including anxiety, schizophrenia, manic depression, delirium, dementia, severe mental retardation, Dentatorubro-pallidoluysian atrophy (DRPLA), Hypophosphatemic rickets, autosomal dominant (2) Acrocallosal syndrome and dyskinesias, such as Huntington's disease or Gilles de la Tourette syndrome and other diseases, disorders and conditions of the like.

NOV35 nucleic acids and polypeptides are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. For example the disclosed NOV35 protein have multiple hydrophilic regions, each of which can be used as an immunogen. This novel protein also has value in development of powerful assay system for functional analysis of various human disorders, which will help in understanding of pathology of the disease and development of new drug targets for various disorders.

NOV36

NOV36 includes two novel cadherin 11-like proteins disclosed below. The disclosed sequences have been named NOV36a and NOV36b.

NOV36a

A disclosed NOV36a nucleic acid of 2476 nucleotides (also referred to as CG56729-01) encoding a novel cadherin 11-like protein is shown in Table 36A. An open reading frame was identified beginning with an ATG initiation codon at nucleotides 46-48 and ending with a TGA codon at nucleotides 2389-2391. Putative untranslated regions, if any, found upstream from the initiation codon and downstream from the termination codon are underlined in Table 36A, and the start and stop codons are in bold letters.

TABLE 36A
NOV36a Nucleotide Sequence

(SEQ ID NO:139)

TCAGCACCCAGGGCCAGTGAACAGAGCCCTGGCTGGAGTCCAAACATGTGGGGCCTGGTGAGGCTCCTGC
TGGCCTGGCTGGGTGGCTGGGGCTGCATGGGGCGTCTGGCAGCCCCAGCCCGGGCCTGGGCAGGGTCCCG
GGAACACCCAGGGCCTGCTCTGCTGCGGACTCGAAGGAGCTGGGTCTGGAACCAGTTCTTTGTCATTGAG
GAATATGCTGGTCCAGAGCCTGTTCTCATTGGCAAGCTGCACTCGGATGTTGACCGGGGAGAGGGCCGCA
CCAAGTACCTGTTGACCGGGGAGGGGGCAGGCACCGTATTTGTGATTGATGAGGCCACAGGCAATATTCA
TGTTACCAAGAGCCTTGACCGGGAGGAAAAGGCGCAATATGTGCTACTGGCCCAAGCCGTGGACCGAGCC
TCCAACCGGCCCCTGGAGCCCCCATCAGAGTTCATCATCAAAGTGCAAGACATCAACGACAATCCACCCA
TTTTTCCCCTTGGGCCCTACCATGCCACCGTGCCCGAGATGTCCAATGTCGGTACATCAGTGATCCAGGT
GACTGCTCACGATGCTGATGACCCCAGCTATGGGAACAGTGCCAAGCTGGTGTACACTGTTCTGGATGGA
CTGCCTTTCTTCTCTGTGGACCCCCAGACTGGTGTGGTGCGTACAGCCATCCCCAACATGGACCGGGAGA
CACAGGAGGAGTTCTTGGTGGTGATCCAGGCCAAGGACATGGGCGGCCACATGGGGGGGCTGTCAGGCAG
CACTACGGTGACTGTCACGCTCAGCCATGTCAACGACAACCCCCCCAAGTTCCCACAGAGTCTATACCAG
TTCTCCGTGGTGGAGACAGCTGGACCTGGCACACTGGTGGGCCGGCTCCGGGCCCAGGACCCAGACCTGG
GGGACAACGCCCTGATGGCATACAGCATCCTGGATGGGGAGGGGTCTGAGGCCTTCAGCATCAGCACAGA
CTTGCAGGGTCGAGACGGGCTCCTCACTGTCCGCAAGGTTCTAGACTTTGAGAGCCAGCGCTCCTACTCC
TTCCGTGTCGAGGCCACCAACACGCTCATTGACCCAGCCTATCTGCGGCGAGGGCCCTTCAAGGATGTGG
CCTCTGTGCGTGTGGCAGTGCAAGATGCCCCAGAGCCACCTGCCTTCACCCAGGCTGCCTACCACCTGAC
AGTGCCTGAGAACAAGGCCCCGGGGACCCTGGTAGGCCAGATCTCCGCGGCTGACCTGGACTCCCCTGCC
AGCCCAATCAGGTACTCCATCCTCCCCCACTCAGATCCGGAGCGTTGCTTCTCTATCCAGCCCGAGGAAG
GCACCATCCATACAGCAGCACCCCTGGATCGCGAGGCTCGCGCCTGGCACAACCTCACTGTGCTGGCTAC
AGAGCTCGGTGAGGACTCCCAGGCCTCGCGCGTGCAAGTGGCCATCCAGACCCTGGATGAGAATGACAAT
GCTCCCCAGCTGGCTGAGCCCTACGATACTTTTGTGTGTGACTCTGCAGCTCCTGGCCAGCTGATTCAGG
TCATCCGGGCCCTGGACAGAGATGAAGTTGGCAACAGTAGCCATGTCTCCTTTCAAGGTCCTCTGGGCCC
TGATGCCAACTTTACTGTCCAGGACAACCGAGATGGCTCCGCCAGCCTGCTGCTGCCCTCCCGCCCTGCT
CCACCCCGCCATGCCCCCTACTTGGTTCCCATAGAACTGTGGGACTGGGGGCAGCCGGCGCTGAGCAGCA
CTGCCACAGTGACTGTTAGTGTGTGCCGCTGCCAGCCTGACGGCTCTGTGGCATCCTGCTGGCCTGAGGC
TCACCTCTCAGCTGCTGGGCTCAGCACCGGCGCCCTGCTTGCCATCATCACCTGTGTGGGTGCCCTCCTT
GCCCTGGTGGTGCTCTTCGTGGCCCTGCGGCGGCAGAAGCAAGAAGCACTGATGGTACTGGAGGAGGAGG
ACGTCCGAGAGAACATCATCACCTACGACGACGAGGGCGGCGGCGAGGAGGACACCGAGGCCTTCGACAT
CACGGCCTTGCAGAACCCGGACGGGGCGGCCCCCCCGGCGCCCGGCCCTCCCGCGCGCCGAGACGTGTTG
CCCCGGGCCCGGGTGTCGCGCCAGCCCAGACCCCCCGGCCCCGCCGACGTGGCGCAGCTCCTGGCGCTGC
GGCTCCGCGAGGCGGACGAGGACCCCGGCGTACCCCCGTACGACTCGGTGCAGGTGTACGGCTACGAGGG
CCGCGGCTCCTCTTGCGGCTCCCTCAGCTCCCTGGGCTCCGGCAGCGAAGCCGGCGGCGCCCCCGGCCCC
GCGGAGCCGCTGGACGACTGGGGTCCGCTCTTCCGCACCCTGGCCGAGCTGTATGGGGCCAAGGAGCCCC
CGGCCCCCTGAGCGCCCGGGCTGGCCCGGCCCACCGCGGGGGGGGGGCAGCGGGCACAGGCCCTCTGAGT
GAGCCCCACGGGGTCCAGGCGGGCGG

The disclosed NOV36a nucleic acid sequence, located on chromosome 14, has 992 of 1514 bases (65%) identical to a gb:GENBANK-ID:HUMCA11A|acc:L34056.1 mRNA from Homo sapiens (cadherin-11 mRNA, complete cds) (E=7.3e⁻¹⁴⁵).

A disclosed NOV36a polypeptide (SEQ ID NO:140) encoded by SEQ ID NO:139 is 781 amino acid residues and is presented using the one-letter amino acid code in Table 36B. Signal P, Psort and/or Hydropathy results predict that NOV36a contains a signal peptide and is likely to be localized in the mitochondrial inner membrane with a certainty of 0.8227 in one embodiment and to the plasma membrane with a certainty of 0.4400 in an additional embodiment. The most likely cleavage site for a NOV36a polypeptide is between amino acids 16 and 17: GWG-CM.

TABLE 36B
Encoded NOV36a protein sequence.

(SEQ ID NO:140)

MWGLVRLLLAWLGGWGCMGRLAAPARAWAGSREHPGPALLRTRRSWVWNQFFVIEEYAGPEPVLIGKLHSDVDRG
EGRTKYLLTGEGAGTVFVIDEATGNIHVTKSLDREEKAQYVLLAQAVDRASNRPLEPPSEFIIKVQDINDNPPIF
PLGPYHATVPEMSNVGTSVIQVTAHDADDPSYGNSAKLVYTVLDGLPFFSVDPQTGVVRTAIPNMDRETQEEFLV
VIQAKDMGGHMGGLSGSTTVTVTLSDVNDNPPKFPQSLYQFSVVETAGPGTLVGRLRAQDPDLGDNALMAYSILD
GEGSEAFSISTDLQGRDGLLTVRKVLDFESQRSYSFRVEATNTLIDPAYLRRGPFKDVASVRVAVQDAPEPPAFT
QAAYHLTVPENKAPGTLVGQISAADLDSPASPIRYSILPHSDPERCFSIQPEEGTIHTAAPLDREARAWHNLTVL
ATELGEDSQASRVQVAIQTLDENDNAPQLAEPYDTFVCDSAAPGQLIQVIRALDRDEVGNSSHVSFQGPLGPDAN
FTVQDNRDGSASLLLPSRPAPPRHAPYLVPIELWDWGQPALSSTATVTVSVCRCQPDGSVASCWPEAHLSAAGLS
TGALLAIITCVGALLALVVLFVALRRQKQEALMVLEEEDVRENIITYDDEGGGEEDTEAFDITALQNPDGAAPPA
PGPPARRDVLPRARVSRQPRPPGPADVAQLLALRLREADEDPGVPPYDSVQVYGYEGRGSSCGSLSSLGSGSEAG
GAPGPAEPLDDWGPLFRTLAELYGAKEPPAP

The disclosed NOV36a amino acid sequence has 434 of 746 amino acid residues (58%) identical to, and 552 of 746 amino acid residues (73%) similar to, the 796 amino acid residue ptnr:SWISSPROT-ACC:P55287 protein from Homo sapiens (Human) (CADHERIN-11 precursor (osteoblast-cadherin) (OB-cadherin) (OSF-4)) (E=2.3e⁻²²⁹).

NOV36a is predicted to be expressed in at least Cerebral Medulla/Cerebral white matter, Gall Bladder, Retina, Temporal Lobe and Uterus. This information was derived by determining the tissue sources of the sequences that were included in the invention including but not limited to SeqCalling sources, Public EST sources, Literature sources, and/or RACE sources.

In addition, NOV36a is predicted to be expressed in the following tissues because of the expression pattern of(GENBANK-ID: gb:GENBANK-ID:HUMCA11A|acc:L34056.1) a closely related Homo sapiens cadherin-11 mRNA, complete cds homolog in species Homo sapiens: osteoblasts.

NOV36b

A disclosed NOV36b nucleic acid of 2476 nucleotides (also referred to as CG56729-02) encoding a novel Cadherin 11-like protein is shown in Table 36C. An open reading frame was identified beginning with an ATG initiation codon at nucleotides 46-48 and ending with a TGA codon at nucleotides 2389-2391. Putative untranslated regions, if any, found upstream from the initiation codon and downstream from the termination codon are underlined in Table 36C, and the start and stop codons are in bold letters.

TABLE 36C
NOV36b Nucleotide Sequence

(SEQ ID.NO:141)

TCAGCACCCAGGGCCAGTGAACAGAGCCCTGGCTGGAGTCCAAACATGTGGGGCCTCGTGAGGCTCCTGC
TGGCCTGGCTGGGTGGCTGGGGCTGCATGGGGCGTCTGGCAGCCCCAGCCCGGGCCTGGGCAGGGTCCCG
GGAACACCCAGGGCCTGCTCTGCTGCGGACTCGAAGCAGCTGGGTCTGGAACCAGTTCTTTGTCATTGAG
GAATATGCTGGTCCAGAGCCTGTTCTCATTGGCAAGCTGCACTCGGATGTTGACCGGGGAGAGGGCCGCA
CCAAGTACCTGTTGACCGGGGAGGGGGCAGGCACCGTATTTGTGATTGATGAGGCCACAGGCAATATTCA
TGTTACCAAGAGCCTTGACCGGGAGGAAAAGGCGCAATATGTGCTACTGGCCCAAGCCGTGGACCGAGCC
TCCAACCGGCCCCTGGAGCCCCCATCAGAGTTCATCATCAAAGTGCAAGACATCAACGACAATCCACCCA
TTTTTCCCCTTGGGCCCTACCATGCCACCGTGCCCGAGATGTCCAATGTCGGTACATCAGTGATCCAGGT
GACTGCTCACGATGCTGATGACCCCAGCTATGGGAACAGTGCCAAGCTGGTGTACACTGTTCTGGATGGA
CTGCCTTTCTTCTCTGTGGACCCCCAGACTGGTGTGGTGCGTACAGCCATCCCCAACATGGACCGGGAGA
CACAGGAGGAGTTCTTGGTGGTGATCCAGGCCAAGGACATGGGCGGCCACATGGGGGGGCTGTCAGGCAG
CACTACGGTGACTGTCACGCTCAGCGATGTCAACGACAACCCCCCCAAGTTCCCACAGAGTCTATACCAG
TTCTCCGTGGTGGAGACAGCTGGACCTGGCACACTGGTGGGCCGGCTCCGGGCCCAGGACCCAGACCTGG
GGGACAACGCCCTGATGGCATACAGCATCCTGGATGGGGAGGGGTCTGAGGCCTTCAGCATCAGCACAGA
CTTGCAGGGTCGAGACGGGCTCCTCACTGTCCGTAAGCCCCTAGACTTTGAGAGCCAGCGCTCCTACTCC
TTCCGTGTCGAGGCCACCAACACGCTCATTGACCCAGCCTATCTGCGGCGAGGGCCCTTCAAGGATGTGG
CCTCTGTGCGTGTGGCAGTGCAAGATGCCCCAGAGCCACCTGCCTTCACCCAGGCTGCCTACCACCTGAC
AGTGCCTGAGAACAAGGCCCCGGGGACCCTGGTAGGCCAGATCTCCGCGGCTGACCTGGACTCCCCTGCC
AGCCCAATCAGGTACTCCATCCTCCCCCACTCAGATCCGGAGCGTTGCTTCTCTATCCAGCCCGAGGAAG
GCACCATCCATACAGCAGCACCCCTGGATCGCGAGGCTCGCGCCTGGCACAACCTCACTGTGCTGGCTAC
AGAGCTCGGTGAGGACTCCCAGGCCTCGCGCGTGCAAGTGGCCATCCAGACCCTGGATGAGAATGACAAT
GCTCCCCAGCTGGCTGAGCCCTACGATACTTTTGTGTGTGACTCTGCAGCTCCTGGCCAGCTGATTCAGG
TCATCCGGGCCCTGGACAGAGATGAAGTTGGCAACAGTAGCCATGTCTCCTTTCAAGGTCCTCTGGGCCC
TGATGCCAACTTTACTGTCCAGGACAACCGAGATGGCTCCGCCAGCCTGCTGCTGCCCTCCCGCCCTGCT
CCACCCCGCCATGCCCCCTACTTGGTTCCCATAGAACTGTGGGACTGGGGGCAGCCGGCGCTGAGCAGCA
CTGCCACAGTGACTGTTAGTGTGTGCCGCTGCCAGCCTGACGGCTCTGTGGCATCCTGCTGGCCTGAGGC
TCACCTCTCAGCTGCTGGGCTCAGCACCGGCGCCCTGCTTGCCATCATCACCTGTGTGGGTGCCCTGCTT
GCCCTGCTGGTGCTCTTCGTGGCCCTGCGGCGGCAGAAGCAAGAAGCACTGATGGTACTGGAGGAGGAGG
ACGTCCGAGAGAACATCATCACCTACGACGACGAGGGCGGCGGCGAGGAGGACACCGAGGCCTTCGACAT
CACGGCCTTGCAGAACCCGGACCGGGCGGCCCCCCCGGCGCCCGGCCCTCCCGCGCGCCGAGACGTGTTC
CCCCGGGCCCGGGTGTCGCGCCAGCCCAGACCCCCCGGCCCCGCCGACGTGGCGCAGCTCCTGGCGCTGC
GGCTCCGCGAGGCGGACGAGGACCCCGGCGTACCCCCGTACGACTCGGTGCAGGTGTACGGCTACGAGGG
CCGCGGCTCCTCTTGCGGCTCCCTCAGCTCCCTGGGCTCCGGCAGCGAAGCCGGCGGCGCCCCCGGCCCC
GCGGAGCCGCTGGACGACTGGGGTCCGCTCTTCCGCACCCTGGCCGAGCTGTATGGGGCCAAGGAGCCCC
CGGCCCCCTGAGCGCCCGGGCTGGCCCGGCCCACCGCGGGGGGGGGGCAGCGGGCACAGGCCCTCTGAGT
GAGCCCCACGGGGTCCAGGCGGGCGG

The disclosed NOV36b nucleic acid sequence, located on chromosome 11, has 1100 of 1109 bases (99%) identical to a gb:GENBANK-ID:AK025342|acc:AK025342.1 mRNA from Homo sapiens (cDNA: FLJ21689 fis, clone COL09459) (E=2.3e⁻²⁴⁰).

A disclosed NOV36b polypeptide (SEQ ID NO:142) encoded by SEQ ID NO:141 is 781 amino acid residues and is presented using the one-letter amino acid code in Table 36D. Signal P, Psort and/or Hydropathy results predict that NOV36b contains a signal peptide and is likely to be localized to the mitochondrial inner membrane with a certainty of 0.8227 in one embodiment and to the plasma membrane with a certainty of 0.4400 in an additional embodiment. The most likely cleavage site for a NOV36b peptide is between amino acids 16 and 17: GWG-CM.

TABLE 36D
Encoded NOV36b protein sequence.

(SEQ ID NO:142)

MWGLVRLLLAWLGGWGCMGRLAAPARAWAGSREHPGPALLRTRRSWVWNQFFVIEEYAGPEPVLIGKLHSDVDRG
EGRTKYLLTGEGAGTVFVIDEATGNIHVTKSLDREEKAQYVLLAQAVDRASNRPLEPPSEFIIKVQDINDNPPIF
PLGPYHATVPEMSNVGTSVIQVTAHDADDPSYGNSAKLVYTVLDGLPFFSVDPQTGVVRTAIPNMDRETQEEFLV
VIQAKDMGGHMGGLSGSTTVTVTLSDVNDNPPKFPQSLYQFSVVETAGPGTLVGRLRAQDPDLGDNALMAYSILD
GEGSEAFSISTDLQGRDGLLTVRKPLDFESQRSYSFRVEATNTLIDPAYLRRGPFKDVASVRVAVQDAPEPPAFT
QAAYHLTVPENKAPGTLVGQISAADLDSPASPIRYSILPHSDPERCFSIQPEEGTIHTAAPLDREARAWHNLTVL
ATELGEDSQASRVQVAIQTLDENDNAPQLAEPYDTFVCDSAAPGQLIQVIRALDRDEVGNSSHVSFQGPLGPDAN
FTVQDNRDGSASLLLPSRPAPPRHAPYLVPIELWDWGQPALSSTATVTVSVCRCQPDGSVASCWPEAHLSAAGLS
TGALLAIITCVGALLALVVLFVALRRQKQEALMVLEEEDVRENIITYDDEGGGEEDTEAFDITALQNPDGAAPPA
PGPPARRDVLPRARVSRQPRPPGPADVAQLLALRLREADEDPGVPPYDSVQVYGYEGRGSSCGSLSSLGSGSEAG
GAPGPAEPLDDWGPLFRTLAELYGAKEPPAP

The disclosed NOV36b amino acid sequence has 435 of 746 amino acid residues (58%) identical to, and 553 of 746 amino acid residues (74%) similar to, the 796 amino acid residue ptnr:SWISSNEW-ACC:P55287 protein from Homo sapiens (Human) (cadherin-11 precursor (osteoblast-cadherin) (OB-cadherin) (OSF-4)) (E=2.5e⁻²³⁰).

NOV36b is predicted to be expressed in at least the following tissues: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea and uterus. Expression information was derived from the tissue sources of the sequences that were included in the derivation of the NOV36b sequence.

In addition, NOV36b is predicted to be expressed in the following tissues because of the expression pattern of (GENBANK-ID: gb:GENBANK-ID:AK025342|acc:AK025342.1) a closely related Homo sapiens cDNA: FLJ21689 fis, clone COL09459 homolog in species Homo sapiens: ostoeblasts.

NOV36a also has homology to the amino acid sequences shown in the BLASTP data listed in Table 36E.

TABLE 36E
BLAST results for NOV36a

| Gene Index/ | | Length | Identity | Positives | |
|---|---|---|---|---|---|
| Identifier | Protein/Organism | (aa) | (%) | (%) | Expect |
| gi\|16553903\|dbj\|BAB71613.1\| | unnamed protein | 493 | 459/466 | 461/466 | 0.0 |
| (AK057922) | product [Homo | | (98%) | (98%) | |
| | sapiens] | | | | |
| gi\|13626134\|sp\|O93319\| | CADHERIN-11 | 792 | 430/749 | 552/749 | 0.0 |
| CADB_CHICK | PRECURSOR | | (57%) | (73%) | |
| gi\|3377485\|gb\|AAC28073.1\| | cadherin | 794 | 429/751 | 547/751 | 0.0 |
| (AF002983) | precursor | | (57%) | (72%) | |
| | [Xenopus laevis] | | | | |
| gi\|1705549\|sp\|P55288\| | CADHERIN-11 | 796 | 432/753 | 552/753 | 0.0 |
| CADB_MOUSE | PRECURSOR | | (57%) | (72%) | |
| | (OSTEOBLAST- | | | | |
| | CADHERIN) (OB- | | | | |
| | CADHERIN) (OSF-4) | | | | |
| gi\|1377894\|dbj\|BAA04798.1\| | OB-cadherin-1 | 796 | 434/753 | 552/753 | 0.0 |
| (D21254) | [Homo sapiens] | | (57%) | (72%) | |

The homology of these sequences is shown graphically in the ClustalW analysis shown in Table 36F.

[image]

[image]

[image]

Tables 36G-P list the domain description from DOMAIN analysis results against NOV36. This indicates that the NOV36 sequence has properties similar to those of other proteins known to contain this domain.

TABLE 36G
Domain Analysis of NOV36

gnl|Pfam|pfam01049, Cadherin_C_term, Cadherin cytoplasmic region.
Cadherins are vital in cell-cell adhesion during tissue
differentiation. Cadherins are linked to the cytoskeleton by catenins.
Catenins bind to the cytoplasmic tail of the cadherin. Cadherins
cluster to form foci of homophilic binding units. A key determinant to
the strength of the binding that it is mediated by cadherins is the
juxtamembrane region of the cadherin. This region induces clustering
and also binds to the protein p120ctn. (SEQ ID NO:827)
CD-Length = 150 residues, 98.7% aligned
Score = 99.8 bits (247), Expect = 5e−22

| | | | |
|---|---|---|---|
| NOV36: | 625 | RRQKQEALMVLEEEDVRENIITYDDEGGGEEDTEAFDITALQNPDGAAPPAPGPPARRDV | 684 |
| | | \|\|+\|+\| \|++ \|+\|\|+\|\|\|\|\| \|\|\|\|\|\|\|\|\|\|\|+\|\|\|\|+\|\|++ \| \| | |
| Sbjct: | 1 | RRRKKEPLIIDEDEDIRENIINYDDEGGGEEDTDAFDISALRSGGNPKPIEELKLRRDIK | 60 |
| NOV36: | 685 | LPRARVSRQPRPPGPADVAQLLALRLREADEDPGVPPYDSVQVYGYEGRGSSCGSLSSLG | 744 |
| | | + \| \|\|\| \| \|+\| + +\|+\|\|\| \|\| \|\|\|\|\|+\| \| \| \|\| + \| | |
| Sbjct: | 61 | PELQSLPRPRRPPAPDDIADFINEKLKEADNDPTAPPYDSLQTYAY--EGSGSVAGSLSS | 118 |
| NOV36: | 745 | SGSEAGGAPGPAEPLDDWGPLFRTLAELYG 774 | |
| | | \| + + \|+\|\|\|\| \|+ \|\|++\|\| | |
| Sbjct: | 119 | LNSSTTDSDQDYDYLNDWGPRFKKLADMYG 148 | |

TABLE 36H
Domain Analysis of NOV36

gnl|Pfam|pfam00028, cadherin, Cadherin domain (SEQ ID NO:828)
CD-Length = 92 residues, 97.8% aligned
Score = 81.3 bits (199), Expect = 2e−16

| | | | |
|---|---|---|---|
| NOV36: | 379 | YHLTVPENKAPGTLVGQISAADLDSPA-SPIRYSILPHSDPERCFSIQPEEGTIHTAAPL | 437 |
| | | \| +\|\|\|\| \|\| \| ++\| \| \| \| \|\|\|\| \| \| \| \|+ \| + \| \|\| | |
| Sbjct: | 1 | YSASVPENAPVGTEVLTVTATDADLGPNGRIFYSILGG-GPGGWFRIDPDTGDLSTTKPL | 59 |
| NOV36: | 438 | DREARAWHNLTVLATELGEDSQASRVQVAIQ | 468 |
| | | \|\|\|+ + \|\|\|\|\|\|+ \| + \| \| | |
| Sbjct: | 60 | DRESIGEYELTVLATDSGGPPLSGTTTVTIT | 90 |

TABLE 36I
Domain Analysis of NOV36

gnl|Pfam|pfam00028, cadherin, Cadherin domain (SEQ ID NO:828)
CD-Length = 92 residues, 100.0% aligned
Score = 72.8 bits (177), Expect = 7e−14

| | | | |
|---|---|---|---|
| NOV36: | 264 | YQFSVVETAGPGTLVGRLRAQDPDLGDNALMAYSILDGEGSEAFSISTDLQGRDGLLTVR | 323 |
| | | \| \|\| \| \| \|\| \| + \| \| \|\|\| \| + \|\|\|\| \| \| \| \| \| \|+ | |
| Sbjct: | 1 | YSASVPENAPVGTEVLTVTATDADLGPNGRIFYSILGGGPGGWFRIDPD----TGDLSTT | 56 |
| | | o | |
| NOV36: | 324 | KVLDFESQRSYSFRVEATNTLIDPAYLRRGPFKDVASVRVAVQ | 366 |
| | | \| \|\| \|\| \| \| \|\|++ \| +\| + \| | |
| Sbjct: | 57 | KPLDRESIGEYELTVLATDSGGPPLS-------GTTTVTITVL | 92 |

TABLE 36J
Domain Analysis of NOV36

gnl|Pfam|pfam00028, cadherin, Cadherin domain (SEQ ID NO:828)
CD-Length = 92 residues, 85.9% aligned
Score = 63.9 bits (154), Expect = 3e−11

| | | | |
|---|---|---|---|
| NOV36: | 155 | YHATVPEMSNVGTSVIQVTAHDADDDPSYGNSAKLVYTVLDGLP-FFSVDPQTGVVRTAI | 212 |
| | | \| \|+\|\|\| + \|\|\| \|+ \|\|\| \|\|\| \| + ++ \|++\| \| \| +\| +\|\| \|\| + \| | |
| Sbjct: | 1 | YSASVPENAPVGTEVLTVTATDAD---LGPNGRIFYSILGGGPGGWFRIDPDTGDLSTTK | 57 |
| NOV36: | 213 | PNNDRETQEEFLVVIQAKDMGGH 235 | |
| | | \| +\|\|\|+ \|+ + + \| \| \|\| | |
| Sbjct: | 58 | P-LDRESIGEYELTVLATDSGGP 79 | |

TABLE 36K
Domain Analysis of NOV36

gnl|Pfam|pfam00028, cadherin, Cadherin domain (SEQ ID NO:828)
CD-Length = 92 residues, 97.8% aligned
Score = 59.3 bits (142), Expect = 8e−10

| | | | |
|---|---|---|---|
| NOV36: | 52 | FVIEEYAGPEPVLIGKLHSDVDRG-EGRTKYLLTGEGAGTVFVIDEATGNIHVTKSLDRE | 110 |
| | | + \| \| ++ +\| \| \| \|\| \| + \| \| \| \| \|\| \|\|++ \|\| \|\|\|\| | |
| Sbjct: | 3 | ASVPENAPVGTEVLTVTATDADLCPNGRIFYSILGGGPGGWFRIDPDTGDLSTTKPLDRE | 62 |
| NOV36: | 111 | EKAQYVLLAQAVDRASNRPLEPPSEFIIKVQ 141 | |
| | | +\| \| \| \| \|\| + \| \| | |
| Sbjct: | 63 | SIGEYELTVLATDSGGPPLSGTTTVTITVL 92 | |

TABLE 36L
Domain Analysis of NOV36

gnl|Pfam|pfam00028, cadherin, Cadherin domain (SEQ ID NO:828)
CD-Length = 92 residues, 100.0% aligned
Score = 44.7 bits (104), Expect = 2e−05

| | | | |
|---|---|---|---|
| NOV42: | 483 | YDTFVCDSAAPGQLIQVIRALDRDEVGNSSHVSFQGPLGPDANFTVQDNRDGSASLLLPS | 542 |
| | | \| \| ++\| \| + + \| \| \| \| \|\| \| + + + \| | |
| Sbjct: | 1 | YSASVPENAPVGTEVLTVTATDADLGPNGRIFYSILGGGPGGWFRIDPD---TGDLSTTK | 57 |
| NOV42: | 543 | RPAPPRHAPYLVPIELWDWGQPALSSTATVTVSVC 577 | |
| | | \| + + \| \| \| \|\| \| \|\|\|++\| | |
| Sbjct: | 58 | PLDRESIGEYELTVLATDSGGPPLSGTTTVTITVL 92 | |

TABLE 36M
Domain Analysis of NOV36

gnl|Smart|smart00112, CA, Cadherin repeats.; Cadherins are (SEQ ID NO:829)
glycoproteins involved in Ca2+-mediated cell-cell adhesion. Cadherin
domains occur as repeats in the extracellular regions which are
thought to mediate cell-cell contact when bound to calcium.
CD-Length = 82 residues, 100.0% aligned
Score = 79.7 bits (195), Expect = 6e−16

| | | | |
|---|---|---|---|
| NOV36: | 396 | ISAADLDSPA-SPIRYSILPHSDPERCFSIQPEEGTIHTAAPLDREARAWHNLTVLATEL | 454 |
| | | +\|\| \| \|\| + \|\|\| + \|\|\| \|\| \| \| \| \|\|\|\|\| ++ + \|\|\| \|\|+ | |
| Sbjct: | 1 | VSATDADSGENGKVTYSILS-GNDGGLFSIDPETGITTTTKPLDREEQSEYTLTVEATDG | 59 |
| NOV36: | 455 | GEDSQASRVQVAIQTLDENDNAP 477 | |
| | | \| +\| \| + \|\| \|\|\|\|\| | |
| Sbjct: | 60 | GCPPLSSTATVTVTVLDVNDNAP 82 | |

TABLE 36N
Domain Analysis of NOV36

gnl|Smart|smart00112, CA, Cadherin repeats.; Cadherins are (SEQ ID NO:829)
glycoproteins involved in Ca2+-mediated cell-cell adhesion. Cadherin
domains occur as repeats in the extracellular regions which are
thought to mediate cell-celDcontact when bound to calcium.
CD-Length = 82 residues, 96.3% aligned
Score = 73.9 bits (180), Expect = 3e−14

| | | | |
|---|---|---|---|
| NOV36: | 70 | SDVDRGE-GRTKYLLTGEGAGTVFVIDEATGNIEVTKSLDREEKAQYVLLAQAVDRASNR | 128 |
| | | +\| \| \|\| \|+ \| + \| +\| \|\| \|\| \| \|\| \|\|\|\|\|+++\| \| +\| \| | |
| Sbjct: | 4 | TDADSGENGKVTYSILSGNDGGLFSIDPETGIITTTKPLDREEQSEYTLTVEATDGGGP- | 62 |
| NOV36: | 129 | PLEPPSEFIIKVQOINDNPP 148 | |
| | | \|\| + + \| \|+\|\|\| \| | |
| Sbjct: | 63 | PLSSTATVTVTVLDVNDNAP 82 | |

TABLE 36O
Domain Analysis of NOV36

gnl|Smart|smart00112, CA, Cadherin repeats.; Cadherins are (SEQ ID NO:829)
glycoproteins involved in Ca2+-mediated cell-cell adhesion. Cadherin
domains occur as repeats in the extracellular regions which are
thought to mediate cell-cell contact when bound to calcium.
CD-Length = 82 residues, 98.8% aligned
Score = 63.5 bits (153), Expect = 4e−11

| | | | |
|---|---|---|---|
| NOV36: | 281 | LRAQDPDLGDNALMAYSILDGEGSEAFSISTDLQORDGLLTVRKVLDFESQRSYSFRVEA | 340 |
| | | + \| \| \| \|+\| + \|\|\|\| \| \|\|\| + \|++\| \| \|\| \| \| \|+ \|\|\| | |
| Sbjct: | 1 | VSATDADSGENGKVTYSILSGNDGGLFSIDPE----TGIITTTKPLDREEQSEYTLTVEA | 56 |
| NOV36: | 341 | TNTLIDPAYLRRGPFKDVASVRVAVQDAPEPP 372 | |
| | | \|+ \| \|+\| \| \| \| + | |
| Sbjct: | 57 | TDGGGP-------PLSSTATVTVTVLDVNDNA 81 | |

TABLE 36P
Domain Analysis of NOV36

gnl|Smart|smart00112, CA, Cadherin repeats.; Cadherins are (SEQ ID NO:829)
glycoproteins involved in Ca2+-mediated cell-cell adhesion. Cadherin
domains occur as repeats in the extracellular regions which are
thought to mediate cell-cell contact when hound to calcium.
CD-Length = 82 residues, 74.4% aligned
Score = 55.8 bits (133), Expect = 9e−09

| | | | |
|---|---|---|---|
| NOV36: | 172 | VTAHDADDPSYGNSAKLVYTVLDGLPF--FSVDPQTGVVRTAIPNMDRETQEEFLVVIQA | 229 |
| | | \|+\| \|\|\| \| \|+ \|++\| \| \|\|+\|\|+\|\|++ \| \| +\|\|\| \| \|+ + ++\| | |
| Sbjct: | 1 | VSATDADSGENGKVTYSILSGNOGGLFSIDPE----TGIITTTKPLDREEQSEYTLTVEA | 56 |
| NOV36: | 230 | KDMGG 234 | |
| | | \| \|\| | |
| Sbjct: | 57 | TDGGG 61 | |
| Sbjct: | 57 | TDGGG 61 | |

Cadherins, first discovered in mouse teratocarcinoma cells, are a family of animal glycoproteins responsible for calcium-dependent cell-cell adhesion. Cadherins preferentially interact with themselves in a homophilic manner in connecting cells; thus acting as both receptor and ligand. There are a number of different isoforms distributed in a tissue-specific manner in a wide variety of organisms. Cells containing different cadherins tend to segregate in vitro, while those that contain the same cadherins tend to preferentially aggregate together. This observation is linked to the finding that cadherin expression causes morphological changes involving the positional segregation of cells into layers, suggesting they may play an important role in the sorting of different cell types during morphogenesis, histogenesis and regeneration. They may also be involved in the regulation of tight and gap junctions, and in the control of intercellular spacing. Cadherins are evolutionary related to the desmogleins which are component of intercellular desmosome junctions involved in the interaction of plaque proteins. The first three cadherins to be described were E-cadherin is present on many types of epithelial cells; N-cadherin on nerve, muscle, and lens cells; and P-cadherin on cells in the placenta and epidermis.

The NOV36 proteins bear close resemblance to cadherin-11, a member of the cadherin family of proteins, expressed in osteoblasts. The tissue expression in brain, uterus and retina of these NOV36 proteins indicate they might play an important role during organogenesis and development.

The disclosed NOV36 nucleic acid of the invention encoding a cadherin 11-like protein includes the nucleic acid whose sequence is provided in Table 36A, 36C or a fragment thereof. The invention also includes a mutant or variant nucleic acid any of whose bases may be changed from the corresponding base shown in Table 36A or 36C while still encoding a protein that maintains its cadherin 11-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, in one embodiment up to about 35% of the NOV36a residues may be so changed and in an additional embodiment up to about 1% of the NOV36b residues may be so changed.

The disclosed NOV36 protein of the invention includes the cadherin 11-like protein whose sequence is provided in Table 36B or 36D. The invention also includes a mutant or variant protein any of whose residues may be changed from the corresponding residue shown in Table 36B or 36D while still encoding a protein that maintains its cadherin 11-like activities and physiological functions, or a functional fragment thereof. In the mutant or variant protein, up to about 43% of the NOV36a and NOV36b bases may be so changed.

The above defined information for this invention suggests that these cadherin 11-like proteins (NOV36) is a member of a “cadherin 11 family”. Therefore, the NOV36 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The nucleic acids and proteins of NOV36 are useful in Von Hippel-Lindau (VHL) syndrome, cirrhosis, transplantation, endometriosis, fertility, anemia, ataxia-telangiectasia, autoimmune disease, hemophilia, hypercoagulation, idiopathic thrombocytopenic purpura, allergies, immunodeficiencies, graft versus host disease (GVHD), lymphaedema, muscular dystrophy, Lesch-Nyhan syndrome, myasthenia gravis, and/or other pathologies and disorders.

NOV36 nucleic acids and polypeptides are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. For example the disclosed NOV36 protein have multiple hydrophilic regions, each of which can be used as an immunogen. This novel protein also has value in development of powerful assay system for functional analysis of various human disorders, which will help in understanding of pathology of the disease and development of new drug targets for various disorders.

NOV37

A disclosed NOV37 nucleic acid of 8575 nucleotides (also referred to as CG56733-01) encoding a novel Ten-M2-like protein is shown in Table 37A. An open reading frame was identified beginning with an ATG initiation codon at nucleotides 199-201 and ending with a TAA codon at nucleotides 8476-8478. Putative untranslated regions, if any, found upstream from the initiation codon and downstream from the termination codon are underlined in Table 37A, and the start and stop codons are in bold letters.

TABLE 37A
NOV37 Nucleotide Sequence

(SEQ ID NO:143)

TAAAGTACCTGTCATCTTGACAAGTGGCGGAGCGGAGGAGTCAAGGATTATAAATGATCACAGCCAAATCC
AGCTCGCCCCGTGATTGGGCTCTCCCGCGATCTGCACCGGGGGAAGCCCATGAGAGGCCAATGAGACTTGA
ACCCTGAGCCTAAGTTGTCACCAGCAGGACTGATGTGCACACAGAAGAGGAATGAAGTATGTGTGAAAGAA
CGCAGGCCTTACTGCTCCCTGACCAAGAGCAGACGAGAGAAGGAACGGCGCTACACAAATTCCTCCGCAGA
CATGAGGAGTGCCGGGTACCCACACAGAAGTCCTACAGTTCCAGCGAGACATTGAAAAGCTTTTGATCATG
ATTCCTCGCGGCTGCTTTACGGCAACAGAGTGAAGGATTTGGTTCACAGAGAAGCAGACGAGTTCACTAGA
CAAAGCAGGATGCACTATGGAAACCGAGTCACAGACCTCATCCACCGGGAGTCAGATGAGTTTCCTAGACA
AGGTATCCTTCACCAGGGCTACTCCCTTAGCACAGGCTCTGACGCCGACTCCGACACCGAGGGAGGGATGT
CTCCAGAACACGCCATCACACTGTGGGGCAGAGGGATAAAATCCAGGCGCAGTTCCGGCCTGTCCAGTCGT
GAAAACTCGGCCCTTACCCTGACTGACTCTGACAACGAAAACAAATCAGATGATGAGAACGGTCGTCCCAT
TCCACGTACATCCTCGCGTAGTCTCCTCCCATTTGTTCAGCTGCCTAGCTCCCATAATCCTCCACCAGTTA
GCTGCCAGATGCCATTGCTAGACAGCAACACCTCCCATCAAATCATGGACACCAACCCTGATGAGGAATTC
TCCCCCAATTCATACCTGCTCAGAGCATGCTCAGGGCCCCAGCAAGCCTCCAGCAGTGGTCCTCCGAACCA
CCACAGCCAGTCGACTCTGAGGCCCCCTCTCCCACCCCCTCACAACCACACGCTGTCCCATCACCACTCGT
CCGCCAACTCCCTCAACAGGAACTCACTGACCAATCGGCGGAGTCAGATCCACGCCCCGGCCCCAGCGCCC
AATGACCTCGCCACCACACCAGAGTCCGTTCAGCTTCACGACAGCTGGGTGCTAAACAGCAACGTGCCACT
GGAGACCCGGCACTTCCTCTTCAAGACCTCCTCGGGGAGCACACCCTTGTTCAGCAGCTCTTCCCCGGGAT
ACCCTTTGACCTCAGGAACGGTTTACACGCCCCCGCCCCGCCTGCTGCCCAGGAATACTTTCTCCAGGAAC
GCTTTCAAGCTGAAGAAGCCCTCCAAATACTGCAGCTCGAAATGTGCTGCCCTCTCCGCCATTGCCGCGGC
CCTCCTCTTGGCTATTTTGCTGGCGTATTTCGCAGCAATGCATCTGCTCGGACTCAATTGGCAACTCCAGC
CTGCAGATGGGCACACCTTTAACAATGGGATAAGGACCGGCTTACCAGGAACGATGATGTGGCAAACAATG
CCATCTGGAGGCAAAGTGCCCTGGTCGTTGAAAACAGCAGCATAAGACAGTGGTGAAACAGAAGTTGGTCG
GCGGGTAACACAACAAGTCCCACCAGGGGTGTTTTGGAGGTCACAAATTCACATCAGTCAGCCCCAGTTCT
TAAAGTTCAACATCTCCCTCGGGAAGGACGCTCTCTTTGGTGTTTACATAAGAAGAGGACTTCCACCATCT
CATGCCCAGTATGACTTCATGGAACGTCTGGACGGGAAAAAGAAGTGGAGTGTGGTTGAGTCTCCCAGGGA
ACGCCGGAGCATACAGACCTTGGTTCAGAATGAAGCCGTGTTTGTGCAGTACCTGGATGTGGGCCTGTGGC
ATCTGGCCTTCTACAATGATGGAAAAGACAAAGAGATGGTTTCCTTCAATACTGTTGTCCTAGATTCAGTG
CACGACTGTCCACGTAACTGCCATGGGAATGGTGAATGTGTGTCCGGGGTGTGTCACTGTTTCCCAGGATT
TCTAGGAGCAGACTGTGCTAAACCTGCCTGCCCTGTCCTGTGCAGTGGGAATGGACAATATTCTAAAGGGA
CGTGCCAGTGCTACAGCGGCTGGAAAGGTGCAGAGTGCGACGTGCCCATGAATCAGTGCATCGATCCTTCC
TGCGGGGGCCACGGCTCCTGCATTGATGGGAACTCTGTCTGCTCTGCTGGCTACAAAGGCGACCACTGTGA
GGAAGTTGATTCCTTGGATCCCACCTGCTCCAGCCACGGAGTCTGTGTGAATGGAGAATGCCTGTGCAGCC
CTGGCTGGGGTGGTCTGAACTGTGAGCTGGCGAGGGTCCAGTGCCCAGACCAGTGCAGTGGGCATGGCACG
TACCTGCCTGACACGGGCCTCTGCAGCTGCGATCCCAACTGGATGGGTCCCCACTGCTCTGTTGTGTGCTC
AGTAGACTGTGGCACTCACGGCGTCTGCATCGGGGGAGCCTGCCGCTGTGAAGAGGGCTAAACAGGCGCAG
CGTGTGACCAGCGCGTGTGCCACCCCCGCTGCATTGAGCACGGGACCTGTAAAGATGGCAAATGTGAATGC
CGAGAGGGCTGGAATGGTGAACACTGCACCATTGATGGCTGCCCTGACTTGTGCAACGGTAACGAAAGATG
CACACTGGGTCAGAACAGCTGGCAGTGTGTCTGCCAGACCGGCTGGACAGGGCCCGGATGCAACGTTGCCA
TGGAAACTTCCTGTGCTGATAACAAGGATAATGAGGGAGATGGACTCATTGACTGCATAAATCCCGATTGC
TGCCTACAGAGTTCCTGCCAGAATCAGCCCTATTGTCGGGGACTGCCGGATCCTCAGGACATCATTAGCCA
AAGCCTTCAATCGCCTTCTCAGCAAGCTGCCAAATCCTTTTATGATCGAATCAGTTTCCTTATAGGATCTG
ATAGCACCCATGTTATACCTGGAGAAAGTCCTTTCAATAGCTTGGTTTCTCTCATCCGAGGCCAAGTAGTA
ACTACAGATGGAACTCCCCTGGTCGGTGTGAACGTGTCTTTTGTCAAGTACCCAAAATACGGCTACACCAT
CACCCGCCAGGATGGCACGTACTCCCTCTCCAGGTTCGACCTGATCGCAAATGGAGGTGCTTCCTTGACTC
TACACTTTGAGCGAOCCCCGTTCATGAGCCAGGAGCGCACTGTGTGGCTGCCGTGGAACAGCTTTTACGCC
ATGGACACCCTGGTGATGAAGACCGAGGAGAACTCCATCCCCAGTTGTGACCTCAGTGGTTTTTGTCGGCT
TGATCCCATCATCATCTCCTCCCCTCTGTCCACTTTCTTTAGTGCTGCCCCTGGGCAGAATCCCATCGTGC
CTGAGACCCAGGTACTTCATGAAGAAATCGAGCTCCCTGGTTCCAATGTGAAACTTCGCTATCTGAGCTCT
AGAACTGCAGGGTACAAGTCACTGCTGAAGATCACCATGACCCAGTCCACAGTGCCCCTGAACCTCATTAG
GGTTCACCTGATGGTGGCTGTCGAGGGGCATCTCTTCCAGAAGTCATTCCAGGCTTCTCCCAACCTGGCCT
ACACCTTCATCTGGGACAAGACAGATCCGTATGGCCAAAGGGTGTATGGACTCTCAGATGCTGTTGGTATG
TTTTGGTTTCAAAGGACAGCCCTCCTTCAGCGATTCGAGCTGGACCCCTCCAACCTCGGTGGCTGGTCCCT
AGACAAACACCACATCCTCAATGTTAAAAGTGGTATCCTACACAAAGGCACTGGGGAAAACCAGTTCCTGA
CCCAGCAGCCTGCCATCATCACCAGCATCATGGGCAATGGTCGCCGCCGCAGCATTTCCTGTCCCAGCTGC
AACGGCCTTGCTGAAGGCAACAAGCTGCTGGCCCCAGTCGCTCTGGCTGTTGGAATCGATGGGAGCCTCTA
TGTGGGTGACTTCAATTACATCCGACGCATCTTTCCCTCTCGAAATGTGACCAGCATCTTGGAGTTACGGA
GAAATAAAGAGTTTAAACATAGCAACAACCCAGCACACAAGTACTACTTGGCAGTGGACCCCGTCTCCGGC
TCGCTCTACGTGTCCGACACCAACAGCAGGAGAATCTACCGCGTCAAGTCTCTGAGTGGAACCAAAGACCT
GGCTGGGAATTCGGAAGTTGTGGCAGGCACGGGAGAGCAGTGTCTACCCTTTGATGAAGCCCGCTGCAAGG
ATGGAGGGAAGCCCATAGATGCAACCCTGATGAGCCCGAGAGGTATTGCAGTAGACAAGAATGGGCTCATG
TACTTTGTCGATGCCACCATGATCCGGAGGTTGACCAGAATGCAATCATCTCCACCCTGCTGGGCTCCAAA
TGACCTCACTGCCGTCCGGCCGCTGAGCTGTGATTCCACCATGGATGTAGCCCAAGTTCGTCTGGAGTGGC
CAACAGACCTTGCTGTCAATCCCATGGATAACTCCTTGTATGTTCTAGAGAACAATGTCATCCTTCGAATC
ACCGAGAACCACCAAGTCAGCATCATTGCGGGACGCCCCATGCACTGCCAAGTTCCTGGCATTGACTACTC
ACTCAGCAAACTAGCCATTCACTCTGCCCTGCAGTCAGCCAGTGCCATTGCCATTTCTCACACTGGGGTCC
TCTACATCACTGAGACAGATGAGAAGAAGATTACCGTCTACGCCAGGTAACAACCAAACGGGGAGATCTGC
CTTTTAGCTGCGGCAGCCTCGGACTGCGACTGCAAAACGATGTCATATTGCAACTGCTATTCAGGAGATGA
TGCCTACGCGACTGATGCCATCTTGAATTCCCCATCATCCTTACCTGTACCTCCAGATGGTACCATTTACA
TTGCAGACCTTGGAATATTCGGATCAGGGCGGTCAGGAGAACAAGCCTGTTCTTAATGCCTTCAAAACCAG
TATGAGGCTGCATCCCCCGGACAGCAGGAGTTATATGTTTTCAACGCTGATGGCATCCACCAATACACTGT
GAGCCTGGTGACAGGGGAGTACTTGTACAATTTCACATATAGTACTGACAATGATGTCACTGAATTGATTG
ACAATAATGGGAATTCCCTGAAGATCCGTCGGGACAGCAGTGGCATGCCCCGTCACCTGCTCATGCCTGAC
AACCAGATCATCACCCTCACCGTGGGCACCAATGGAGGCCTCAAAGTCGTGTCCACACAGAACCTGGAGCT
TGGTCTCATGACCTATGATGGCAACACTGCGCTCCTGGCCACCAAGAGCGATGAAACAGGATGGACGACTT
TCTATAGCTATGACCACGAAGGCCGCCTGACCAACGTGACGCGCCCCACGGGGGTGGTAACCAGTCTCCAC
CGGGAAATGGAGAAATCTATTACCATTGACATTGAGAACTCCAACCGTGATGATGACGTCACTGTCATCAC
CAACCTCTCTTCAGTAGAGGCCTCCTACACAGTGGTACAAGATCAAGTTCGGAACAGCTACCAGCTCTGTA
ATAATGGTACCCTGAGGGTGATGTATGCTAATGGGATGGGTATCAGCTTCCACAGCGAGCCCCATGTCCTA
GCGGGCACCATCACCCCCACCATTGGACGCTGCAACATCTCCCTGCCTATGGAGAATGGCTTAAACTCCAT
TGAGTGGCGCCTAAGAAAGGAACAGATTAAACGCAAAGTCACCATCTTTGGCAGGAAGCTCGAGGTCCATG
GAAGAAATCTCTTGTCCATTGACTATGATCGAAATATTCGGACTGAAAGATCTATGATGACCACCAAAAAG
TTCACCCTGAGGATCATTTATGACCAGGTGGGCCGCCCCTTCCTCTGGCTGCCCAGCAGCGGGCTGGCAGC
TGTCAACGTGTCATACTTCTTCAATGGCCGCCTGGCTGGGCTTCAGCGTGGGGCCATGAGCGAGAGGACAG
ACATCCACAAGCAAGGCCGCATCGTGTCCCGCATGTTCCCTGACGGGAAAGTGTGGAGCTACTCCTACCTT
GACAACATGGTCCTCCTGCTTCAGAGCCAACCTCAGTATATATTTGAGTATGACTCCTCTGACCGCCTCCT
TGCCGTCACCATGCCCAGCGTGGCCCGGCACAGCATCTCCACACACACCTCCATCGGCTACATCCGTAATA
TTTACAACCCGCCTGAAGCAATGCTTCGGTCATCTTTGACTACAAGTGATGACGGCCGCATCCTGAAGACC
TCCTTTTTGGGCACCGGACGCCAGGTGTTCTACAAGTATGGGAAACTCTCCAAGTTATCAGAGATTGTCTA
CGACAGTACCGCCGTCACCTTCCGGTATGACGAGACCACTGGTGTCTTGAAGATGGTCAAACCTCCAAGTG
GGGGCTTCTCCTGCACCATCAGCTACCGGAAGATTGGCCCCCTGGTGGACAAGCAGATCTACAGGTTCTCC
GAGGAAGGCATGGTCAATGCCAGGTTTGACTACACCTATCATGACAACAGCTTCCGCATCGCAAGCATCAA
GCCCGTCATAAGTGAGACTCCCCTCCCCGTTGACCTCTACCGCTATGATGAGATTTCTGGCAGGTGGAAAC
ACTTTGGTAAGTTTGGAGTCATCTATTATGACATCAACCAGATCATCACCACTGCCGTGATGACCCTCAGC
AAACACTTCGACACCCATGGGCGCATCAAGGAGGTCCAGTATGAGATGTTCCGGTCCCTCATGTACTGGAT
GACCGTGCAATATGACAGCATGGGCAGGGTGATCAAGAGGGAGCTAAAACTGGGGCCCTATOCCAATACCA
CGAAGTACACCTATGACTACGATGGGGACGGGCAGCTCCAGAGCGTCCCGGCCGTCTAACACCGCCCGACC
TGGCGCTACAGCTATGACCTTAATGGGAATCTCCACTTACTGAACCCAGGCAACAGTGTGCGCCTCATGCC
CTTGCCCTATGACCTCCGGGATCGGATAACCAGACTCGGGGATGTGCAGTACAAAATTGACGACGATGGCT
ATCTGTGCCAGAGAGGGTCTGACATCTTCGAATACAATTCCAAGGGCCTCCTAACAAGAGCCTACAACAAG
GCCAGCGGGTGGAGTGTCCAGTACCGCTATGATCGCGTAGGACGGCGGGCTTCCTACAAGACCAACCTGGG
CCACCACCTGCAGTACTTCTACTCTGACCTCCACAACCCGACGCGCATCACCCATGTCTACAATCACTCCA
ACTCGGAGATTACCTCACTGTACTACGACCTCCAGGGCCACCTCTTTGCCATGGAGACCAGCAGTGGGGAG
CAGTACTATGTTGCCTCTGATAACACAGGGACTCCTCTGGCTGTGTTCAGCATCAACGGCCTCATGATCAA
ACAGCTGCAGTACACGGCCTATGGGGAGATTTATTATGACTCCAACCCCGACTTCCAGATGGTCATTGGCT
TCCATGGGGGACTCTATGACCCCCTGACCAAGCTGGTCCACTTCACTCAGCGTGATTATGATGTGCTGGCA
GGACGATGGACCTCCCCAGACTATACCATGTGGAAAAACGTGGGCAAGGAGCCGGCCCCCTTTAACCTGTA
TATGTTCAAGAGCAACAATCCTCTCAGCAGTGAGCTAGATTTGAAGAACTACGTOACAGATGTGTAAAGCT
GGCTTGTGATGTTTGGATTTCAGCTTAGCAACATCATTCCTGGCTTCCCGAGACCCAAAATGTATTTCGTG
CCTCCTCCCTATGAATTGTCAGAGAGTCAAGCAAGTGAGAATGGACAGCTCATCACAGGTGCCCACCAGAC
AACAGAGAGACATAACCAGGCTTTCATGGCTCTGGAAGGACAGGTCATTACTAAAAAGCTCCACGCCAGCA
TCCGAGAGAAAGCAGGTCATTGGTTTGCCACCACCACGCCCATCATTGGCAAAGGCATCATGTTTGCCATC
AAAGAAGGGCCGGTGACCACGGGCGTGTCCAGCATCGCCAGCGAAGATAGCCGCAAGGTCGCATCTGTCCT
GACAACGCCTACTACCTGGACAAGATGCAACTACAGCATCGAGGGCAAGGACACCCACTACTTTGTGAAGA
TTGGCTCAGCCGATCGCGACCTGGTCACACTAGGCACCACCATCGGCCGCAAGGTGCTACAGAGCGGGGTG
AACGTGACCGTGTCCCAGCCCACGCTGCTGGTCAACGGCAGGACTCGAACGTTCACGAACATTGAGTTCCA
GTACTCCACGCTGCTGCTCAGCATCCGCTATGGCCTCACCCCCGACACCCTGGACGAAGAGAAGGCCCGCG
TCCTGGACCAGGCGAGACAGAGGGCCCTGGGCACGGCCTGGGCCAAGGAGCAGCAGAAAGCCAGGGACGGG
AGAGAGGGGAGCCGCCTGTGGACTGAGGGCGAGAAGCAGCAGCTTCTGAGCACCGGGCGCGTGCAAGGCTA
CGAGGGATATTACGTGCTTCCCGTGGAGCAATACCCAGAGCTTGCAGACAGTACCAGCAACATCCAGTTTT
TAAGACAGAATGAGATGGGAAGAGGTAACAAAATTAATCTGCTGCCATTCCTTGTCTGAATGGCTCAGCAG
GAGTAACTGTTATCTCCTCTCCTAAGGAGATGAAGACCTACAGGGGCACTGCGG

The NOV37 nucleic acid, located on chromosome 5, has 4965 of 5004 bases (99%) identical to a gb:GENBANK-ID:AB032953|acc:AB032953.1 mRNA from Homo sapiens (mRNA for KIAA1127 protein, partial cds) (E=0.0).

A disclosed NOV37 polypeptide (SEQ ID NO:144) encoded by SEQ ID NO:143 is 2759 amino acid residues and is presented using the one-letter code in Table 37B. Signal P, Psort and/or Hydropathy results predict that NOV37 does not contain a signal peptide and is likely to be localized extracellularly with a certainty of 0.7900 in one embodiment, to the plasma membrane with a certainty of 0.7900 in another embodiment and to the nucleus with a certainty of 0.6000 in an additional embodiment.

TABLE 37B
Encoded NOV37 protein sequence

(SEQ ID NO:144)

MDVKERRPYCSLTKSRREKERRYTNSSADNEECRVPTQKSYSSSETLAAJFDHDSSRLLYGNRVKLVHREAD
EFTRQSRMHYGNRVTDLIHRESDEFPRQGILHQGYSLSTGSDADSDTEGGRVISPEHAIRLWGRGTKSRRSSGL
SSRENSALTLTDSDNENKSDDENGRPIPRTSSRSLLPFVQLPSSHNPPPVSCQMPLLDSNTSHQIMDTNPDE
EFSPNSYLLRACSGPQQASSSGPPNHTSQSTLRPPLPPPITHHTLSHHHSSPRSLNRNSLTNRRSQIHTAPAPA
PNDLATTPESVQLQDSWVLNSNVPLETRHFLFKTSSGSTPLFSSSSPCJYPLTSGTVYTPPPRLLPRNTFSRK
AFKLKKPSKYCSWKCAISAIALLLAILLAYFAAAHLLGLNWQLQPAAAGHTFAAGIRTGLPCNDDVATMP
SGGKVPWSLKNSSIDSGEAEVGRRVTQEVPPGVFWRSQIHISQPQFLKFNISLGKDALFGVYIRRGLPPSHA
QYDFMERLDCKEKWSVVESPRERRSIQTLVQNEAVFVQYLDVGLWHLAFYNDGKDKEMVSFNTVVLDSVQDC
PRCHGNGECVSGVCHCFPCFLGADCAKAJCPVLCSGNGQYSKGTCQCYSGWKGAECDVPAAJQCIDPSCGGH
GSCIDCNCVCSAGYKGEHCEEVDCLIJPTCSSHGVCVNCECLCSPGWGGLNCELARVQCPDQCSGHGTYLPDT
GLCSCDPNWMGPDCSVVCSVDCGTHCVCIGGACRCEEGWTGAACDQRVCHPRCIEHGTCKDGKCECREGWNG
EHCTIDGCPDLCNGNGRCTLGQNSWQCVCQTGWRGPGCNVAIAAIETSCADNKDNEGDCLIDCMDPDCCLQSSCQ
NQPYCRGLPDPQDIISQSLQSPSQQAAKSFYDRISFLIGSDSTHVIPGESPFNSLVSLIRCIQVVTTDGTPLV
GVNVSFVKYPKYGYTITRQDGTYSLSRFDLIANGGASLTLHFERAPFMSQERTVWLPWNSFYAMDTLVMKTE
ENSIPSCDLSGFCRLDPIIISSPLSTFFSAAPCIQNPIVPETQVLHEETELPGSNVKLRYLSSRTAGYKSLLK
ITMTQSTVPLNLIRVHLMVAVEGHLFQKSFQASPNLAYTFIWDKTDAYGQRVYGLSDAVGMFWFQRTALLQG
FELDPSNLGGWSLDKHHILNVKSGILHKGTGENQFLTQQPAIITSIMCNGRRRSISCPSCNGLAEGNKLLAP
VALAVGIDGSLYVGDFNYIRRIFPSRNVTSILELRRNKEFKHSNNPAHKYYLAVDPVSGSLYVSDTNSRRIY
RVKSLSGTKDLAGNSEVVAGTGEQCLPFDEARCGDSGKAIDATLMSPRGIAVDKNGLMYFVDATMIRKVDQN
GIISTLLCSNDLTAVRPLSCDSSMDVAQVRLEWPTDLAVNPMDNSLYVLENNVILRITENHQVSIIAGRPDH
CQVPGIDYSLSKLAIHSALESASAIAISHTGVLYITETDEKKINRLRQVTTNGEICLLAGAASDCDCKNDVN
CNCYSGDDAYATDAILNSPSSLAVAPDGTIYIADLGNIRIRAVSKNKPVLNAFNQYEAASPGEQELYVFNAD
GIHQYTVSLVTGEYLYNFTYSTDNDVTELIDNNGNSLKIRRDSSGMPRHLLMPDNQIITLTVGTNGGLKVVS
TQNLELGLMTYDGNTGLLATKSDETGWTTFYSYDHEGRLTNVTRPTGVVTSLHREMEKSITIDIENSNRDDD
VTVITNLSSVEASYTVVQDQVRNSYQLCNNGTLRVMYAAGMGISFHSEPHVLAGTITPTIGRCNISLPMENG
LNSIEWRLRKEQIKGKVTIFGRKLEVHGRNLLSIDYDRNIRTEKIYDDHRKFTLRIIYDQVGRPFLWLPSSG
LAAVNVSYFFNGRLAGLQRGAIVISERTDIDKQGRIVSRMFAIGKVWSYSYLDKLLLQSQRQYIFEYDSSDR
LLAVTMPSVARHSMSTHTSIGYIRNIYNPPESNASVIFDYSDDGRILKTSFLGTCRQVFYKYGKLSKLSEIV
YDSTAVTFGYDETTGVLKMVNLQSGGFSCTIRYRKIGPLVDKQIYRFSEEGMVNARFDYTYHDNSFRIASLK
PVISETPLPVDLYRYDEISGKVEHFGKFGVIYYDINQIITTAVMTLSKHFDTHGRIKEVQYEMFRSLMYWMT
VQYDSMGRVIKRELKLGPYANTTKYTYDYDGDGQLQSVPAVNDRPTWRYSYDLNGNLHLLNPGNSVRLMPLR
YDLRDRITRLGDVQYKIDDDGYLCQRGSDIFEYNSKGLLTRAYNKASGWSVQYRYDGVGRRASYKTNLGHHL
QYFYSDLHNPTRITHVYNHSNSEITSLYYDLQGHLFANESSSGEEYYVASDNTGTPLAVFSINGLMIKQLQY
TAYGEIYYDSNPDFQMVIGFHGGLYDPLTKLVHFTQRDYDVLAGRWTSPDYTMWKMVGKEPAPFNLYMFKSN
NPLSSELDLKNYVTDVKSWLVMFGFQLSNIIPGFPRAKMYFVPPPYELSESQASENGQLITGAHQTTERHNQ
AFMALEGQVITKKLHASIREKAGHWFATTTPIIGKGIMFAIKEGRVTTGVSSIASEDSRKVASVLNNAYYLD
KMHYSIEGKDTHYFVKIGSADGDLVTLGTTIGRKVLESGVNVTVSQPTLLVNGRTRRFTNIEFQYSTLLLSI
RYGLTPDTLDEEKARSTIDQARQRALGTAWAKEQQKARDGREGSRLWTEGEKQQLLSTGRVQGYEGYYVLPVE
QYPELADSSSNIQFLRQNEMGKR

The disclosed NOV37 amino acid sequence has 2634 of 2764 amino acid residues (95%) identical to, and 2679 of 2764 amino acid residues (96%) similar to, the 2764 amino acid residue ptnr:SPTREMBL-ACC:Q9WTS5 protein from Mus musculus (Mouse) (TEN-M2) (E=0.0).

NOV37 is predicted to be expressed in at least Amygdala, Brain, Bronchus, Cerebral Medulla/Cerebral white matter, Cochlea, Coronary Artery, Epidermis, Hair Follicles, Hippocampus, Hypothalamus, Kidney, Left cerebellum, Lung, Lymph node, Parietal Lobe, Pineal Gland, Retina, Right Cerebellum, Substantia Nigra, Vulva and Whole Organism. This information was derived by determining the tissue sources of the sequences that were included in the invention including but not limited to SeqCalling sources, Public EST sources, Literature sources, and/or RACE sources.

In addition, NOV37 is predicted to be expressed in the following tissues because of the expression pattern of (GENBANK-ID: gb:GENBANK-ID:AB032953|acc: AB032953.1) a closely related Homo sapiens mRNA for KIAA1127 protein, partial cds homolog in species Homo sapiens: Amygdala, Brain, Bronchus, Cerebral Medulla/Cerebral white matter, Cochlea, Coronary Artery, Epidermis, Hair Follicles, Hippocampus, Hypothalamus, Kidney, Left cerebellum, Lung, Lymph node, Parietal Lobe, Pineal Gland, Retina, Right Cerebellum and Substantia Nigra.

NOV37 also has homology to the amino acid sequences shown in the BLASTP data listed in Table 37C.

TABLE 37C
BLAST results for NOV37

| Gene Index/ | | Length | Identity | Positives | |
|---|---|---|---|---|---|
| Identifier | Protein/Organism | (aa) | (%) | (%) | Expect |
| gi\|7657415\|ref\|NP_035986.2\| | odd Oz/ten-m | 2764 | 2633/2777 | 2677/2777 | 0.0 |
| (NM_011856) | homolog 2 | | (94%) | (95%) | |
| | (Drosophila); odd | | | | |
| | Oz/ten-m homolog | | | | |
| | 3 (Drosophila) | | | | |
| | [Mus musculus] | | | | |
| gi\|9910320\|ref\|NP_064473.1\| | neurestin alpha | 2765 | 2625/2778 | 2676/2778 | 0.0 |
| (NM_020088) | [Rattus | | (94%) | (95%) | |
| | norvegicus] | | | | |
| gi\|10241574\|emb\|CAC09416.1\| | teneurin-2 | 2802 | 2525/2818 | 2639/2818 | 0.0 |
| (AJ279031) | [Gallus gallus] | | (89%) | (93%) | |
| gi\|5307761\|dbj\|BAA81892.1\| | ten-m3 [Danio | 2590 | 1707/2602 | 2097/2602 | 0.0 |
| (AB026979) | rerio] | | (65%) | (79%) | |
| gi\|6760369\|gb\|AAF28316.1\| | ODZ3 [Mus | 2346 | 1665/2360 | 1993/2360 | 0.0 |
| AF195418_1 | musculus] | | (70%) | (83%) | |
| (AF195418) | | | | | |

The homology of these sequences is shown graphically in the ClustalW analysis shown in Table 37D.

[image]

[image]

[image]

[image]

[image]

[image]

[image]

[image]

Table 37E lists the domain description from DOMAIN analysis results against NOV37. This indicates that the NOV37 sequence has properties similar to those of other proteins known to contain this domain.

TABLE 37E
Domain Analysis of NOV37

gnl|Pfam|pfam02068, Metallothio_PEC, Plant PEC family metallothionein.
(SEQ ID NO:830)
CD-Length = 77 residues, 97.4% aligned
Score = 41.6 bits (96), Expect = 6e−04

| | | | |
|---|---|---|---|
| NOV37: | 738 | CSVDCGTHGVCIGG-ACRCEEGWTGAACDQRVCHPRCIEHGTCKDGKCECREGWNGEHCT | 796 |
| | | \| \|\| \| \|\| +\|\|\| \| \| \|\| \|\| \|\| \| \|\|\|\| | |
| Sbjct: | 2 | CDDKCGCPSPCPGGNSCRCTSGGEAGAGDQ--------EHTTC---PC-------GEHC- | 42 |
| NOV37: | 797 | IDGC-PDLCNGNGRCTLGQNSWQCVCQTGWRGPCCNVANETSCA 839 | |
| | | \|\| \| \| \| + \| \| \| \|\| \| \|\|\| | |
| Sbjct: | 43 | --GCNPCTCPKTQTPTGRKGRANCSC-----GAGCTCA---SCA 76 | |

Neurestin shows homology to a neuregulin gene product, human gamma-heregulin, a Drosophila receptor-type pair-rule gene product, Odd Oz (Odz)/Ten(m), and Ten(a), suggesting a possible function in synapse formation and morphogenesis. A mouse neurestin homolog has independently been cloned as DOC4 from the NIH-3T3 cell line. Northern blot analysis showed that neurestin is highly expressed in the brain and also in other tissues at much lower levels. In situ hybridization studies showed that neurestin is expressed in many types of neurons, including pyramidal cells in the cerebral cortex and tufted cells in the olfactory bulb during development. In adults, neurestin is mainly expressed in olfactory and hippocampal granule cells, which are known to be generated throughout adulthood. Nonetheless, in adults the expression of neurestin was experimentally induced in external tufted cells during regeneration of olfactory sensory neurons. These results suggest a role for neurestin in neuronal development and regeneration in the central nervous system

Neurestin is a putative transmembrane protein whose expression is developmentally regulated in neurons. Neurestin expression pattern were examined in mitral/tufted cells in the developing rat olfactory bulb. In the main olfactory bulb, neurestin expression was segregated in the dorso-rostral area and in the ventro-caudal area, but not in between. In the accessory olfactory bulb, neurestin expression was found only in the far caudal area. This area did not completely correspond to a caudal half of the vomeronasal nerve and glomerular layers positive for a G-protein Go alpha. These spatio-temporal expression patterns suggest that neurestin functions as a target recognition molecule that specifies zonal projection patterns of olfactory and vomeronasal sensory neurons

The NOV37 nucleic acid of the invention encoding a Ten-M2-like protein includes the nucleic acid whose sequence is provided in Table 37A, or a fragment thereof. The invention also includes a mutant or variant nucleic acid any of whose bases may be changed from the corresponding base shown in Table 37A while still encoding a protein that maintains its Ten-M2-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 1% of the residues may be so changed.

The NOV37 protein of the invention includes the Ten-M2-like protein whose sequence is provided in Table 37B. The invention also includes a mutant or variant protein any of whose residues may be changed from the corresponding residue shown in Table 37B while still encoding a protein that maintains its Ten-M2-like activities and physiological functions, or a functional fragment thereof. In the mutant or variant protein, up to about 35% of the bases may be so changed.

The NOV37 nucleic acids and proteins of the invention are useful in potential diagnostic and therapeutic applications implicated in various diseases and disorders described below and/or other pathologies. For example, the compositions of the present invention will have efficacy for treatment of patients suffering from: brain disorders including epilepsy, eating disorders, schizophrenia, ADD, cancer, heart disease, inflammation and autoimmune disorders including Crohn's disease, IBD, allergies, rheumatoid and osteoarthritis, inflammatory skin disorders, allergies, blood disorders, psoriasis, colon cancer, leukemia, AIDS, thalamus disorders, metabolic disorders including diabetes and obesity, lung diseases such as asthma, emphysema, cystic fibrosis, and cancer, pancreatic disorders including pancreatic insufficiency and cancer, and prostate disorders including prostate cancer and other diseases, disorders and conditions of the like.

NOV37 nucleic acids and polypeptides are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. For example the disclosed NOV37 protein have multiple hydrophilic regions, each of which can be used as an immunogen. This novel protein also has value in development of powerful assay system for functional analysis of various human disorders, which will help in understanding of pathology of the disease and development of new drug targets for various disorders.

NOV38

A disclosed NOV38 nucleic acid of 1090 nucleotides (also referred to as CG56737-01) encoding a novel Activin Beta C Chain-like protein is shown in Table 38A. An open reading frame was identified beginning with an ATG initiation codon at nucleotides 3-5 and ending with a TAG codon at nucleotides 1068-1070. Putative untranslated regions, if any, found upstream from the initiation codon and downstream from the termination codon are underlined in Table 38A, and the start and stop codons are in bold letters.

TABLE 38A
NOV38 Nucleotide Sequence

(SEQ ID NO:145)

CAATGACCTCCTCATTGCTTCTGGCCTTTCTCCTCCTGGCTCCAACCACAGTGGCCACTCCCAGAGCTGG
CGGTCAGTGTCCAGCATGTGGGGGGCCCACCTTGGAACTGGAGAGCCAGCGGGAGCTGCTTCTTGATCTG
GCCAAGAGAAGCATCTTGGACAAGCTGCACCTCACCCAGCGCCCAACACTGAACCGCCCTGTGTCCAGAG
CTGCTTTGAGGACTGCACTGCAGCACCTCCACGGGGTCCCACAGGGCGCACTTCTAGACGACAACAGGGA
ACAGGAATGTGAAATCATCAGCTTTGCTGAGACAGACTCCACTTCAGCCTACAGCTCCCTGCTCACTTTT
CACCTGTCCACTCCTCGGTCCCACCACCTGTACCATGCCCGCCTGTGGCTGCACGTGCTCCCCACCCTTC
CTGGCACTCTTTGCTTGAGGATCTTCCGATGCGGACCAAGGAGGAGGCGCCAAGGGTCCCGCACTCTCCT
GGCTGAGCACCACATCACCAACCTGGGCTGGCATACCTTAACTCTGCCCTCTAGTGGCTTGAGGGGTGAG
AAGTCCGGTGTCCTGAAACTGCAACTAGACTGCAGACCCCTAGAAGGCAACAGCACAGTTACTGCACAAC
CGAGCCGGCTCTTGGACACAGCAGGACACCAGCAGCCCTTCCTAGAGCTAAAGATCCGAGCCAATGAGCC
TGGACCAGGCCGGGCCAGGAGGAGGACCCCCACCTGTGAGCCTGCGACCCCCTTATGTTGCAGGCGAGAC
CATTACGTAGACTTCCAGGAACTGGCATGGCGGGACTGGATACTGCAGCCCGAGGGGTACCAGCTGAATT
ACTGCAGTGCGCAGTGCCCTCCCCACCTGGCTGGCACCCCAGGCATTGCTGCCTCTTTCCATTCTGCCGT
CTTCAGCCTCCTCAAAGCCAACAATCCTTGGCCTGCCACTACCTCCTGTTGTGTCCCTACTGCCCGAAGG
CCCCTCTCTCTCCTCTACCTGGATCATAGTGGCAATGTGGTCAAGACGGATGTGCCAGATATGGTGGTGG
AGGCCTGTGGCTGCACCTAGCAAGAGGACCTGGGGCTTTG

The disclosed NOV38 nucleic acid sequence, located on chromosome 12q13.1, has 748 of 935 bases (80%) identical to a gb:GENBANK-ID:MMU96386|acc:U96386.1 mRNA from Mus musculus (activin beta E subunit mRNA, complete cds) (E=5.2e⁻¹²⁰).

A disclosed NOV38 polypeptide (SEQ ID NO:146) encoded by SEQ ID NO:145 is 355 amino acid residues and is presented using the one-letter amino acid code in Table 38B. Signal P, Psort and/or Hydropathy results predict that NOV38 contains a signal peptide and is likely to be localized extracellularly with a certainty of 0.5135. The most likely cleavage site for a NOV38 peptide is between amino acids 18 and 19: TVA-TP .

TABLE 38B
Encoded NOV38 protein sequence.

(SEQ ID NO:146)

MTSSLLLAFLLLAPTTVATPRAGGQCPACGGPTLELESQRELLLDLAKRSILDKLNLTQRPTLNRPVSRAALRTA
LQHLHGVPQGALLEDNREQECEIISFAETDSTSAYSSLLTFHLSTPRSHHLYHARLWLHVLPTLPGTLCLRIFRW
GPRRRRQCSRTLLAEHHITNLGWHTLTLPSSGLRGEKSGVLKLQLDCRPLEGNSTVTGQPRRLLDTAGHQQPFLE
LKIRANEPGAGRARRRTPTCEPATPLCCRRDHYVDFQELGWRDWILQPEGYQLNYCSGQCPPHLAGSPGIAASFH
SAVFSLLKANNPWPASTSCCVPTARRPLSLLYLDHSGNVVKTDVPDMVVEACGCS

The disclosed NOV38 amino acid sequence has 217 of 355 amino acid residues (61%) identical to, and 253 of 355 amino acid residues (71%) similar to, the 352 amino acid residue ptnr:SWISSPROT-ACC:P55103 protein from Homo sapiens (Human) (inhibin beta C chain precursor (activin beta-C chain)) (E=7.6e⁻¹⁰³).

NOV38 is predicted to be expressed in at least ovary and liver. This information was derived by determining the tissue sources of the sequences that were included in the invention including but not limited to SeqCalling sources, Public EST sources, Literature sources, and/or RACE sources.

Possible small nucleotide polymorphisms (SNPs) found for NOV38 are listed in Table 38C.

TABLE 38C
SNPs

| Consensus | | Base | |
|---|---|---|---|
| Position | Depth | Change | PAF |
| 95 | 14 | T > C | N/A |

NOV38 also has homology to the amino acid sequences shown in the BLASTP data listed in Table 38D.

TABLE 38D
BLAST results for NOV38

| Gene Index/ | | Length | Identity | Positives | |
|---|---|---|---|---|---|
| Identifier | Protein/Organism | (aa) | (%) | (%) | Expect |
| gi\|13899338\|ref\|NP_{—} | hypothetical | 350 | 291/348 | 307/348 | e−153 |
| 113667.1\| | protein MGC4638 | | (83%) | (87%) | |
| (NM_031479) | [Homo sapiens] | | | | |
| gi\|14714539\|gb\|AAH10404.1\| | inhibin beta E | 350 | 253/352 | 280/352 | e−129 |
| AAH10404 | [Mus musculus] | | (71%) | (78%) | |
| (BC010404) | | | | | |
| gi\|6680453\|ref\|NP_032408.1\| | inhibin beta E | 350 | 253/352 | 280/352 | e−129 |
| (NM_008382) | [Mus musculus] | | (71%) | (78%) | |
| gi\|13929160\|ref\|NP_{—} | activin beta E | 350 | 250/352 | 279/352 | e−125 |
| 114003.1\| | [Rattus | | (71%) | (79%) | |
| (NM_031815) | norvegicus] | | | | |
| gi\|4809189\|gb\|AAD30133.1\| | activin beta E | 350 | 248/352 | 279/352 | e−124 |
| AF140032_1 | [Rattus | | (70%) | (78%) | |
| (AF140032) | norvegicus] | | | | |

The homology of these sequences is shown graphically in the ClustalW analysis shown in Table 38E.

[image]

[image]

Tables 38F-G list the domain description from DOMAIN analysis results against NOV38. This indicates that the NOV38 sequence has properties similar to those of other proteins known to contain this domain.

TABLE 38F
Domain Analysis of NOV38

gnL|Smart|smart00204, TGFB, Transforming growth factor-beta (TGF-beta)
family; Family members are active as disuiphide-linked-homo- or
heterodimers. TGFB is a multifunctional peptide that controls
proliferation, differentiation, and other functions in many cell types
(SEQ ID NO:831)
CD-Length = 102 residues, 100.0% aligned
Score = 134 hits (336), Expect = 1e−32

| | | | |
|---|---|---|---|
| NOV38: | 252 | CCRRDHYVDFQELGWRDWILQPEGYQLNYCSGQCPPHLAGSPGIAASFHSAVFSLLKANN | 311 |
| | | \| \| \| \|\|\|\|++\|\|\| \|\|\|+ \|+\|\| \|\| \|+\|\| \|+ \|+ \|+ \| \|\|+ \| + | |
| Sbjct: | 1 | CRRHDLYVDFKDLGWDDWIIAPKGYNAYYCEGECPFPLSERLN--ATNHAIVQSLVHALD | 58 |
| NOV38: | 312 | PWPASTSCCVPTARRPLSLLYLDHSGNVVKTDVPDMVVEACGCS 355 | |
| | | \| \|\|\|\|\| \|\|\|+\|\| \| \|\|\|\| + \| \|\|\|\| \|\|\| | |
| Sbjct: | 59 | PGAVPKPCCVPTKLSPLSMLYYDDDGNVVLRNYPNMVVEECGCR 102 | |

TABLE 38G
Domain Analysis of NOV38

gnl|Pfam|pfam00019, TGF-beta, Transforming growth factor beta like
domain (SEQ ID NO:832)
CD-Length = 105 residues, 97.1% aligned
Score = 114 bits (286), Expect = 7e−27

| | | | |
|---|---|---|---|
| NOV38: | 252 | CCRRDHYVDFQELGWRDWILQPEGYQLNYCSGQCPPHLAGSPGIAASFHSAVFSLLKANN | 311 |
| | | \| \| \|\|\|\|++\|\|\| \|\|\|+ \|\|\|\| \|\|\|\|\| \|\| \| \| \|+ + +\|++ \| | |
| Sbjct: | 4 | CRLRSLYVDFRDLGWGDWIIAPEGYIANYCSGSCPFPLRDDLN--LSNHAILQTLVRLRN | 61 |
| NOV38: | 312 | PWPASTSCCVPTARRPLSLLYLDHSGNVVKTDVPDMVVEACGCS 355 | |
| | | \| \|\|\|\|\| \|\|\|+\|\|\|\| + \|\|\| \|+\| \|+ \|\|\| | |
| Sbjct: | 62 | PRAVPQPCCVPTXLSPLSMLYLDDNSNVVLRLYPNMSVKECGCR 105 | |

Activins are homo- or heterodimers of related beta subunits (see 147290) while inhibins are dimers composed of an alpha subunit (147380) and an activin beta subunit (summarized in Schmitt et al., Genomics 1996, 32(3):358-66). Activin proteins belong to the TGF-beta superfamily (see 190180), the members of which have important roles in cell determination, differentiation, and growth.

TGFB is a multifunctional peptide that controls proliferation, differentiation, and other functions in many cell types. It was first identified by its ability to cause phenotypic transformation of rat fibroblasts. TGFB is chemically distinct from TGFA. It has essentially no sequence homology with TGFA or with epidermal growth factor, of which TGFA is an analog. Members of the same gene family as TGFB include inhibin, which inhibits pituitary secretion of follicle stimulating hormone, and Mullerian inhibitory substance, which is produced by the testis and is responsible for regression of the Mullerian ducts (anlagen of the female reproductive system) in the male embryo. Many cells synthesize TGFB and almost all of them have specific receptors for this peptide. Alpha and beta TGFs are classes of transforming growth factors. TGFB acts synergistically with TGFA in inducing transformation. It also acts as a negative autocrine growth factor.

TGF-beta plays an important role in wound healing. A number of pathologic conditions, such as idiopathic pulmonary fibrosis, scleroderma, and keloids, which share the characteristic of fibrosis, are associated with increased TGF-beta-1 expression.

The disclosed NOV38 nucleic acid of the invention encoding a Activin Beta C Chain-like protein includes the nucleic acid whose sequence is provided in Table 38A or a fragment thereof. The invention also includes a mutant or variant nucleic acid any of whose bases may be changed from the corresponding base shown in Table 38A while still encoding a protein that maintains its Activin Beta C Chain-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, in one embodiment up to about 20% of the NOV38 residues may be so changed.

The disclosed NOV38 protein of the invention includes the Activin Beta C Chain-like protein whose sequence is provided in Table 38B. The invention also includes a mutant or variant protein any of whose residues may be changed from the corresponding residue shown in Table 38B while still encoding a protein that maintains its Activin Beta C Chain-like activities and physiological functions, or a functional fragment thereof. In one embodiment a mutant or variant protein of NOV38, up to about 39% of the bases may be so changed.

The above defined information for this invention suggests that these Activin Beta C Chain-like proteins (NOV38) is a member of a “Activin Beta C Chain family” . Therefore, the NOV38 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The nucleic acids and proteins of NOV38 are useful in Alzheimer disease-5, Myxoid liposarcoma, Stickler syndrome, type I (3), SED, Alpha-ketoglutarate dehydrogenase deficiency, Cerebral cavernous malformations-2, Greig cephalopolysyndactyly syndrome, Hyperinsulinism, familial, MODY, type 2, Pallister-Hall syndrome, Polydactyly, postaxial, types A1 and B, Polydactyly, postaxial, type IV, Retinitis pigmentosa-9, Charcot-Marie-Tooth neuropathy-2D, Colton blood group, Deafness, autosomal dominant 5, Macular dystrophy, dominant cystoid, Radioulnar synostosis with amegakaryocytic thrombocytopenia, Von Hippel-Lindau (VHL) syndrome, cirrhosis, transplantation, fertility and/or other pathologies and disorders.

NOV38 nucleic acids and polypeptides are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. For example the disclosed NOV38 protein have multiple hydrophilic regions, each of which can be used as an immunogen. This novel protein also has value in development of powerful assay system for functional analysis of various human disorders, which will help in understanding of pathology of the disease and development of new drug targets for various disorders.

NOV39

NOV39 includes novel Activin Beta C Chain-like and Inhibin Beta E Chain Precursor-like proteins disclosed below. The disclosed sequences have been named NOV39a and NOV39b.

NOV39a

A disclosed NOV39a nucleic acid of 1112 nucleotides (also referred to as CG56737-02) encoding a novel Inhibin Beta E Chain Precursor-like protein is shown in Table 39A. An open reading frame was identified beginning with an ATG initiation codon at nucleotides 40-42 and ending with a TAG codon at nucleotides 1090-1092. Putative untranslated regions, if any, found upstream from the initiation codon and downstream from the termination codon are underlined in Table 39A, and the start and stop codons are in bold letters.

TABLE 39A
NOV39a Nucleotide Sequence

(SEQ ID NO:147)

CCATCCGAGGCTCCTGAACCGGGGCCATTCACCAGGAGCATGCGGCTCCCTGATGTCCAGCTCTGGCTGG
TGCTGCTGTGGGCACTGGTGCGAGCACAGGGGACAGGGTCTGTGTGTCCCTCCTGTGGGGGCTCCAAACT
GCCACCCCAAGCAGAACGAGCTCTGGTGCTGGAGCTAGCCAAGCAGCAAATCCTGGATGGGTTGCACCTG
ACCAGTCGTCCCAGAATAACTCATCCTCCACCCCAGGCAGCGCTGACCAGAGCCCTCCGGAGACTACAGC
CAGGGAGTGTGGCTCCAGGGAATGGGGAGCAGGTCATCAGCTTTGCTACTGTCACAGACTCCACTTCAGC
CTACAGCTCCCTGCTCACTTTTCACCTGTCCACTCCTCCGTCCCACCACCTGTACCATGCCCGCCTGTGG
CTGCACGTGCTCCCCACCCTTCCTGGCACTCTTTGCTTGAGGATCTTCCGATGGGGACCAAGGAGGAGGC
GCCAAGGGTCCCGCACTCTCCTGGCTGAGCACCACATCACCAACCTGGGCTGGCATACCTTAACTCTGCC
CTCTAGTGGCTTGAGGGGTGAGAAGTCTGGTGTCCTGAAACTGCAACTAGACTGCAGACCCCTAGAAGGC
AACAGCACAGTTACTGGACAACCGAGGCGGCTCTTGGACACAGCAGCACACCAGCAGCCCTTCCTAGAGC
TTAAGATCCGAGCCAATGAGCCTGGAGCAGGCCGGGCCAGGAGGAGGACCCCCACCTGTGAGCCTGCGAC
CCCCTTATGTTGCAGGCGAGACCATTACCTAGACTTCCAGGAACTGGGATGGCGGGACTGGATACTGCAG
CCCGAGGGGTACCAGCTGAATTACTGCAGTGGGCAGTGCCCTCCCCACCCGGCTGGCAGCCCAGGCATTG
CTGCCTCTTTCCATTCTGCCGTCTTCAGCCTCCTCAAAGCCAACAATCCTTGGCCTGCCAGTACCTCCTG
TTGTGTCCCTACTGCCCGAAGGCCCCTCTCTCTCCTCTACCTCGATCATAATGCCAATGTGGTCAAGACG
GATGTGCCAGATATGGTGGTGGAGGCCTGTGGCTGCAGCTAGCAAGAGGACCTGGGGCTTTG

The disclosed NOV39a nucleic acid sequence, located on chromosome 7p13-15, has 923 of 1110 bases (83%) identical to a gb:GENBANK-ID:MMU96386|acc:U96386.1 mRNA from Mus musculus (activin beta E subunit mRNA, complete cds) (E=2.1e⁻¹⁶⁵).

A disclosed NOV39a polypeptide (SEQ ID NO:148) encoded by SEQ ID NO:147 is 350 amino acid residues and is presented using the one-letter amino acid code in Table 39B. Signal P, Psort and/or Hydropathy results predict that NOV39a contains no signal peptide and is likely to be localized extracellularly with a certainty of 0.3700. The most likely cleavage site for a NOV39a peptide is between amino acids 19 and 20: VRA-QG.

TABLE 39B
Encoded NOV39a protein sequence.

(SEQ ID NO:148)

MRLPDVQLWLVLLWALVRAQGTGSVCPSCGGSKLAPQAERALVLELAKQQILDGLHLTSRPRITHPPPQAALTRA
LRRLQPGSVAPGNGEEVISFATVTDSTSAYSSLLTFHLSTPRSHHLYHARLWLHVLPTLPGTLCLRIFRWGPRRR
RQGSRTLLAEHHITNLGWHTLTLPSSGLRGEKSGVLKLQLDCRPLEGNSTVTGQPRRLLDTAGHQQPFLELKIRA
NEPGAGRARRRTPTCEPATPLCCRRHYVDFQELGWRDWILQPEGYQLNYCSGQCPPHPAGSPGIAASFHSAVFS
LLKANNPWPASTSCCVPTARRPLSLLYLDHNGNVVKTDVPDMVVEACGCS

The disclosed NOV39a amino acid sequence has 287 of 350 amino acid residues (82%) identical to, and 301 of 350 amino acid residues (86%) similar to, the 350 amino acid residue ptnr:SWISSPROT-ACC:O08717 protein from Mus musculus (Mouse) (Inhibin Beta E Chain Precursor (Activin Beta-E Chain)) (E=5.0e⁻¹⁵⁴).

NOV39a is predicted to be expressed in at least the following tissues: ovary and liver. Expression information was derived from the tissue sources of the sequences that were included in the derivation of the NOV39a sequence.

NOV39b

A disclosed NOV39b nucleic acid of 1112 nucleotides (also referred to as CG56647-03) encoding a novel Inhibin Beta E Chain-like protein is shown in Table 39C. An open reading frame was identified beginning with an ATG initiation codon at nucleotides 40-42 and ending with a TAG codon at nucleotides 1090-1092. Putative untranslated regions, if any, found upstream from the initiation codon and downstream from the termination codon are underlined in Table 39C, and the start and stop codons are in bold letters.

TABLE 39C
NOV39b Nucleotide Sequence

(SEQ ID NO:149)

CCATCCGAGGCTCCTGAACCGGGGCCATTCACCAGGAGCATGCGGCTCCCTGATGTCCAGCTCTGGCTGG
TGCTGCTGTGGGCACTGGTGCGAGCACAGGGGACAGGGTCTGTGTGTCCCTCCTGTGGGGGCTCCAAACT
GGCACCCCAAGCAGAACGAGCTCTGGTGCTGGAGCTAGCCAAGCAGCAAATCCTGGATGGGTTGCACCTG
ACCAGTCGTCCCAGAATAACTCATCTTCCACCCCAGGCAGCGCTGACCAGAGCCCTCCGGAGACTACAGC
CAGGGAGTGTGGCTCCAGGGAATGGGGAGGAGGTCATCAGCTTTGCTACTGTCACAGACTCCACTTCAGC
CTACAGCTCCCTGCTCACTTTTCACCTGTCCACTCCTCCGTCCCACCACCTGTACCATCCCCGCCTGTGG
CTGCACGTGCTCCCCACCCTTCCTGGCACTCTTTGCTTGAGGATCTTCCGATGGGGACCAAGGAGGAGGC
GCCAAGGGTCCCGCACTCTCCTGGCTGAGCACCACATCACCAACCTGGGCTGGCATACCTTAACTCTGCC
CTCTAGTGGCTTGAGGGGTGAGAAGTCTGGTGTCCTGAAACTGCAACTAGACTGCAGACCCCTAGAAGGC
AACAGCACAGTTACTGGACAACCGAGGCGGCTCTTGGACACAGCAGGACACCAGCAGCCCTTCCTAGAGC
TTAAGATCCGAGCCAATGAGCCTGGAGCAGGCCGGGCCAGGAGGGGGACCCCCACCTGTGAGCCCGCGAC
CCCCTTATGTTGCAGGCGAGACCATTACGTAGACTTCCAGGAACTGGGATGGCGGGACTGGATACTGCAG
CCCGAGGGGTACCAGCTGAATTACTGCAGTGGGCAGTGCCCTCCCCACCTGGCTGGCAGCCCAGGCATTG
CTGTCTCTTTCCATTCTGCCGTCTTCAGCCTCCTCAAAGCCAACAATCCTTGGCCTGCCAGTACCTCCTG
TTGTGTCCCTACTGCCCGAAGGCCCCTCTCTCTCCTCTACCTGGATCATAATGGCAATGTGGTCAAGACG
GATGTGCCAGATATGGTGGTGGAGGCCTGTGGCTGCAGCTAGCAAGAGGACCTGGGGCTTTG

The disclosed NOV39b nucleic acid sequence, located on chromosome 7p13-15, has 920 of 1110 bases (82%) identical to a gb:GENBANK-ID:MMU96386|acc:U96386.1 mRNA from Mus musculus (activin beta E subunit mRNA, complete cds) (E=3.7e⁻¹⁶⁴).

A disclosed NOV39b polypeptide (SEQ ID NO:150) encoded by SEQ ID NO:149 is 350 amino acid residues and is presented using the one-letter amino acid code in Table 39D. Signal P, Psort and/or Hydropathy results predict that NOV39b contains a signal peptide and is likely to be localized extracellularly with a certainty of 0.3700. The most likely cleavage site for a NOV39b peptide is between amino acids 19 and 20: VRA-QG.

TABLE 39D
Encoded NOV39b protein sequence.

(SEQ ID NO:150)

MRLPDVQLWLVLLWALVRAQGTGSVCPSCGGSKLAPQAERALVLELAKQQILDGLHLTSRPRITHLPPQAALTRA
LRRLQPGSVAPGNGEEVISFATVTDSTSAYSSLLTFHLSTPRSHHLYHARLWLHVLPTLPGTLCLRIFRWGPRRR
RQGSRTLLAEHHITNLGWHTLTLPSSGLRGEKSGVLKLQLDCRPLEGNSTVTGQPRRLLDTAGHQQPFLELKIRA
NEPGAGRARRGTPTCEPATPLCCRRDHYVDFQELGWRDWILQPEGYQLNYCSGQCPPHLAGSPGIAVSFHSAVFS
LLKANNPWPASTSCCVPTARRPLSLLYLDHNGNVVKTDVPDMVVEACGCS

The disclosed NOV39b amino acid sequence has 285 of 350 amino acid residues (81%) identical to, and 299 of 350 amino acid residues (85%) similar to, the 350 amino acid residue ptnr:SWISSPROT-ACC:O08717 protein from Mus musculus (Mouse) (Inhibin Beta E Chain Precursor (Activin Beta-E Chain)) (E=1.5e⁻¹⁵²).

NOV39b is predicted to be expressed in at least the following tissues: ovary and liver. Expression information was derived from the tissue sources of the sequences that were included in the derivation of the NOV39b sequence.

Possible small nucleotide polymorphisms (SNPs) found for NOV39a are listed in Table 39E.

TABLE 39E
SNPs

| Consensus | | Base | |
|---|---|---|---|
| Position | Depth | Change | PAF |
| 1095 | 11 | T > C | N/A |

Possible small nucleotide polymorphisms (SNPs) found for NOV39b are listed in Table 39F.

TABLE 39F
SNPs

| Consensus | | Base | |
|---|---|---|---|
| Position | Depth | Change | PAF |
| 933 | 9 | C > T | 0.222 |

NOV39a also has homology to the amino acid sequences shown in the BLASTP data listed in Table 39G.

TABLE 39G
BLAST results for NOV39a

| Gene Index/ | | Length | Identity | Positives | |
|---|---|---|---|---|---|
| Identifier | Protein/Organism | (aa) | (%) | (%) | Expect |
| gi\|13899338\|ref\|NP_{—} | hypothetical | 350 | 349/350 | 349/350 | 0.0 |
| 113667.1\| | protein MGC4638 | | (99%) | (99%) | |
| (NM_031479) | [Homo sapiens] | | | | |
| gi\|14714539\|gb\|AAH10404.1\| | inhibin beta E | 350 | 288/351 | 302/351 | e−153 |
| AAH10404 | [Mus musculus] | | (82%) | (85%) | |
| (BC010404) | | | | | |
| gi\|6680453\|ref\|NP_032408.1\| | inhibin beta E | 350 | 287/351 | 301/351 | e−152 |
| (NM_008382) | [Mus musculus] | | (81%) | (84%) | |
| gi\|13929160\|ref\|NP_{—} | activin beta E | 350 | 281/351 | 294/351 | e−146 |
| 114003.1\| | [Rattus | | (80%) | (83%) | |
| (NM_031815) | norvegicus] | | | | |
| gi\|4809189\|gb\|AAD30133.1\| | activin beta E | 350 | 279/351 | 294/351 | e−146 |
| AF140032_1 | [Rattus | | (79%) | (83%) | |
| (AF140032) | norvegicus] | | | | |

The homology of these sequences is shown graphically in the ClustalW analysis shown in Table 39H.

[image]

[image]

Tables 39I-J list the domain description from DOMAIN analysis results against NOV39. This indicates that the NOV39 sequence has properties similar to those of other proteins known to contain this domain.

TABLE 39I
Domain Analysis of NOV39

gnl|smart|smart00204, TGFB, Transforming growth factor-beta (TGF-beta)
family; Family members are active as disuiphide-linked homo- or
heterodimers. TGFB is a multifunctional peptide that controls
proliferation, differentiation, and other functions in many cell
types. (SEQ ID NO:833)
CD-Length = 102 residues, 100.0% aligned
Score = 133 bits (335), Expect = 1e−32

| | | | |
|---|---|---|---|
| NOV39: | 247 | CCRRDHYVDFQELGWRDWILQPEGYQLNYCSGQCPPHPAGSPGIAASFHSAVFSLLKANN | 306 |
| | | \| \| \| \|\|\|\|++\|\|\| \|\|\|+ \|+\|\| \|\| \|+\|\| + \|+ \|+ \| \|\|+ \| + | |
| Sbjct: | 1 | CRRHDLYVDFKDLGWDDWIIAPKGYNAYYCEGECPFPLSERLN--ATNHAIVQSLVHALD | 58 |
| NOV39: | 307 | PWPASTSCCVPTARRPLSLLYLDHNGNVVKTDVPDMVVEACGCS 350 | |
| | | \| \|\|\|\|\| \|\|\|+\|\| \| +\|\|\|\| + \|+\|\|\|\| \|\|\|\| | |
| Sbjct: | 59 | PGAVPKPCCVPTKLSPLSMLYYDDDGNVVLRNYPNMVVEECGCR 102 | |

TABLE 39J
Domain Analysis of NOV39

gnl|Pfam|pfam00019, TGF-beta, Transforming growth factor beta like
domain. (SEQ ID NO:834)
CD-Length = 105 residues, 97.1% aligned
Score = 114 bits (286), Expect = 7e−27

| | | | |
|---|---|---|---|
| NOV46: | 247 | CCRRDHYVDFQELGWRDWILQPEGYQLNYCSGQCPPHPAGSPGIAASFHSAVFSLLKANN | 306 |
| | | \| \| \|\|\|\|++\|\|\| \|\|\|+ \|\|\|\| \|\|\|\|\| \|\| \| \|+ + +\|++ \| | |
| Sbjct: | 4 | CRLRSLYVDFRDLGWGDWIIAPEGYIANYCSGSCPFPLRDDLN--LSNHAILQTLVRLRN | 61 |
| NOV46: | 307 | PWPASTSCCVPTARRPLSLLYLDHNGNVVKTDVPDMVVEACGCS 350 | |
| | | \| \|\|\|\|\| \|\|\|+\|\|\|\| \| \|\|\| \|+\| \|+ \|\|\| | |
| Sbjct: | 62 | PRAVPQPCCVPTKLSPLSMLYLDDNSNVVLRLYPNMSVKECGCR 105 | |

Activins are homo- or heterodimers of related beta subunits (see 147290) while inhibins are dimers composed of an alpha subunit (147380) and an activin beta subunit (summarized in Schmitt et al., Genomics 1996, 32(3):358-66). Activin proteins belong to the TGF-beta superfamily (see 190180), the members of which have important roles in cell determination, differentiation, and growth.

TGFB is a multifunctional peptide that controls proliferation, differentiation, and other functions in many cell types. It was first identified by its ability to cause phenotypic transformation of rat fibroblasts. TGFB is chemically distinct from TGFA. It has essentially no sequence homology with TGFA or with epidermal growth factor, of which TGFA is an analog. Members of the same gene family as TGFB include inhibin, which inhibits pituitary secretion of follicle stimulating hormone, and Mullerian inhibitory substance, which is produced by the testis and is responsible for regression of the Mullerian ducts (anlagen of the female reproductive system) in the male embryo. Many cells synthesize TGFB and almost all of them have specific receptors for this peptide. Alpha and beta TGFs are classes of transforming growth factors. TGFB acts synergistically with TGFA in inducing transformation. It also acts as a negative autocrine growth factor.

TGF-beta plays an important role in wound healing. A number of pathologic conditions, such as idiopathic pulmonary fibrosis, scleroderma, and keloids, which share the characteristic of fibrosis, are associated with increased TGF-beta-I expression.

The disclosed NOV39 nucleic acid of the invention encoding a Activin Beta C Chain-like protein includes the nucleic acid whose sequence is provided in Table 39A, 39C or a fragment thereof. The invention also includes a mutant or variant nucleic acid any of whose bases may be changed from the corresponding base shown in Table 39A, or 39C while still encoding a protein that maintains its Activin Beta C Chain-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, in one embodiment up to about 17% of the NOV39a residues may be so changed, and in an additional embodiment up to about 18% of the NOV39b residues may be so changed.

The disclosed NOV39 protein of the invention includes the Activin Beta C Chain-like protein whose sequence is provided in Table 39B, or 39D. The invention also includes a mutant or variant protein any of whose residues may be changed from the corresponding residue shown in Table 39B, or 39D while still encoding a protein that maintains its Activin Beta C Chain-like activities and physiological functions, or a functional fragment thereof. In one embodiment a mutant or variant protein of NOV39a, up to about 39% of the bases may be so changed.

The above defined information for this invention suggests that these Activin Beta C Chain-like proteins (NOV39) is a member of a “Activin Beta C Chain family”. Therefore, the NOV39 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The nucleic acids and proteins of NOV39 are useful in Alzheimer disease-5, Myxoid liposarcoma, Stickler syndrome, type I (3), SED, Alpha-ketoglutarate dehydrogenase deficiency, Cerebral cavernous malformations-2, Greig cephalopolysyndactyly syndrome, Hyperinsulinism, familial, MODY, type 2, Pallister-Hall syndrome, Polydactyly, postaxial, types A1 and B, Polydactyly, postaxial, type IV, Retinitis pigmentosa-9, Charcot-Marie-Tooth neuropathy-2D, Colton blood group, Deafness, autosomal dominant 5, Macular dystrophy, dominant cystoid, Radioulnar synostosis with amegakaryocytic thrombocytopenia, Von Hippel-Lindau (VHL) syndrome, cirrhosis, transplantation, fertility and/or other pathologies and disorders.

NOV39 nucleic acids and polypeptides are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. For example the disclosed NOV39 protein have multiple hydrophilic regions, each of which can be used as an immunogen. This novel protein also has value in development of powerful assay system for functional analysis of various human disorders, which will help in understanding of pathology of the disease and development of new drug targets for various disorders.

NOV40

A disclosed NOV40 nucleic acid of 1606 nucleotides (also referred to as CG56097-01) encoding a UDP glycosyltransferase-like protein is shown in Table 40A. An open reading frame was identified beginning with a ATG initiation codon at nucleotides 1-3 and ending with a TAG codon at nucleotides 1600-1602. The start and stop codons are shown in bold in Table 40A, and the 5′ and 3′ untranslated regions, if any, are underlined.

TABLE 40A
NOV40 nucleotide sequence.

(SEQ IB NO:151)

ATGGCTATGAAATGGACTTCAGTCCTTCTCTTGATACAGCTGAGCTATTACTCTAGCTCTGGGAGTTGTGGA
AATGTGCCGCTGTGGCCCATGGAATATAGTCCTTGGATGAATATAAAGACAATCCTGGATAAACTTATGCAG
ATAAGTCATGAGGTGACTGTTCTAACATTGTCAGCTTCCATTCTTGTTGATCCCAACATAACATCTGTTACT
AAATTTGAGGTTTATTCTATATCTGTAATTAAAGATGATTTTGCAGGGTTTTTTTTCACACAACAGATTACT
AAATGGATACATGATCTTCCAAAACATATATTTTGGTTTAAATGTGTTCCCTTCAAGAATATTCTTTGGGAA
TATTCTGGTTATACTGAGAAGTTCTTTAAAGATGTAGTTTTGAACAAGAAACTTATGACAAACCTACAAGAA
TCAAGGTCTGATGTCGTTCATGCAAATGCCATTGGTCCCTTTGGAGAGCTGCTGGCTGAGCTATTAAAAATA
TCCTTTGTGTACAGTCTCCACTTCTCTCCTGGCTACACATTTGAGAAATACAGTGGAGGATTTCTACTTCCA
CCTTCCTATGGAGCTGTTATTCTGTCAGAATTAAGTGGTTCGATGACATTCATGGAGACAGTAAGAAATATT
ATATATGTGTTTTATTTTGACTTTTGGTTCCAAACATTTGATATGAAGAAGGGAGACCAGTTTTACAGTGAA
GTTCTAGGTAAGTCATGTTTTTTATCTGAGATAATGGGAAAAGCTGAAATGTGGCTCATTCGAAACTACTGG
TATTTGGAATTTCCTCGCCCACTCTTACCTAATTTTGAATTTGTTGTAAGACTCTACTGCAAACCTGTCAAC
CCCCTGCCTAAGGAGAAAATGGAAGAATTTGCCCAGAGCTCTGATGAAGACGGTGTTGTGTTTTCTCTGGAG
TCAGCTGTGCAAAACCTTACAGAAGAAAAAGCTGATCTTATCACTTCGGCCCTGGCTCAGATTCCACAAAAA
GTCATGAAGTTCGGAAGGAAACCAAATACCTTAAGATCCAATACTCAGTGGCATAGGTGGATCCCACAGAAT
GAATGTCTTATCCTAGATCATCCCCAAACCAAAGCCTTTATAACTTATGGTGGAACAAATAGCATCTATGAG
ATGATCTACCGTGGAGTCCCTTCCATGGGCATTCCTTTGTTTGCGGACCAACATGATAACATTGCTCACATG
AAGGCCAAGGGAGCAGCTGTTATATTGGACTTGAGCACAAAGTCAAGTACAGATTTGCTCGATATATCTGTG
TTCGTATCTTTATTTTTATCCTTCAGATATAAAGAGAGTGTTATGAAATTATCAAGAATTCAACATGATCAA
CCAGTGAAGCCCCTGGATCGAGCAGTCTTCTGGATTGAATTTGTCATGCGCCACAAAGGAGCCAAACACCTT
CGAGTTGCAGCCCGTGACCTCACCTGGTTCCAGTACCACTCTTTGGATGTGATTGGGTTTCTGCTGGCCTGT
GTGGCAACTGTGACATTTATCATCACAAAGTGTTGTCTGTTTTGTTTCTGGAAGTTTACTAGAAAAGTGAAG
AAGGAAAAAAGGGATTAGTTAT

In a search of public sequence databases, the NOV40 nucleic acid sequence, located on chromosome 4, has 1305 of 1606 bases (81%) identical to a gb:GENBANK-ID:HUMUDPGTA|acc:J05428.1 mRNA from Homo sapiens (Human 3,4-catechol estrogen UDP-glucuronosyltransferase mRNA, complete cds) (E=6.4e⁻²¹⁷).

The disclosed NOV40 polypeptide (SEQ ID NO:152) encoded by SEQ ID NO:151 has 533 amino acid residues and is presented in Table 40B using the one-letter amino acid code. Signal P, Psort and/or Hydropathy results predict that NOV40 has no signal peptide and is likely to be localized to the endoplasmic reticulum (membrane) with a certainty of 0.8200. Alternatively, NOV40 may also localize to the plasma membrane with a certainty of 0.4600, to the microbody (peroxisome) with a certainty of 0.3012, or to the lysosome (membrane) with a certainty of 0.2000. The most likely cleavage site for NOV40 is between positions 20 and 21: SSS-GS.

TABLE 40B
Encoded NOV40 protein sequence.

(SEQ ID NO:152)

MAMKWTSVLLLIQLSYYSSSGSCGNVPLWPMEYSPWMNIKTILDKLMQISHEVTVLTLSASILVDPNITSVT
KFEVYSISVIKDDFAGFFFTQQITKWIHDLPKHTFWFKCVPFKNILWEYSGYTEKFFKDVVLNKKLMTNLQE
SRSDVVHANAIGPFGELLAELLKISFVYSLHFSPGYTFEKYSGGFLLPPSYGAVILSELSGSMTFMETVRNI
IYVFYFDFWFQTFDMKKGDQFYSEVLGKSCFLSEIMGKAEMWLTRNYWYLEFPRPLLPNFEFVVRLYCKPVN
PLPKEKMEEFAQSSDEDGVVFSLESAVQNLTEEKADLITSALAQIPQKVMKFGRKPNTLRSNTQWHRWIPQN
ECLILDHPQTKAFITYGGTNSIYEMIYRGVPSMGIPLFADQHDNIAHMKAKGAAVILDLSTKSSTDLLDISV
FVSLFLSFRYKESVMKLSRIQHDQPVKPLDRAVFWIEFVMRHKGAKHLRVAARDLTWFQYHSLDVIGFLLAC
VATVTFIITKCCLFCFWKFTRKVKKEKRD

A search of sequence databases reveals that the NOV40 amino acid sequence has 353 of 533 amino acid residues (66%) identical to, and 412 of 533 amino acid residues (77%) similar to, the 529 amino acid residue ptnr:SWISSPROT-ACC:P16662 protein from Homo sapiens (Human) (UDP-Glucuronosyltransferase 2b7 Precursor, Microsomal (EC 2.4.1.17) (UDPGT) (3,4-Catechol Estrogen Specific) (UDPGTH-2)) (E=7.2e⁻¹⁸⁵),

NOV40 is predicted to be expressed in at least the following tissues: liver tissue. This information was derived by determining the tissue sources of the sequences that were included in the invention including but not limited to SeqCalling sources, Public EST sources, Literature sources, and/or RACE sources.

NOV40 also has homology to the amino acid sequences shown in the BLASTP data listed in Table 40C.

TABLE 40C
BLAST results for NOV40

| Gene Index/ | | Length | Identity | Positives | |
|---|---|---|---|---|---|
| Identifier | Protein/Organism | (aa) | (%) | (%) | Expect |
| gi\|4507825\|ref\|NP_001065.1\| | UDP | 529 | 353/536 | 412/536 | 0.0 |
| (NM_001074) | glycosyltransferase | | (65%) | (76%) | |
| | 2 family, | | | | |
| | polypeptide B7 | | | | |
| gi\|6175083\|sp\|P06133\| | UDP- | 528 | 355/536 | 409/536 | 0.0 |
| UDB4_HUMAN | GLUCURONOSYLTRANSFERASE | | (66%) | (76%) | |
| | 2B4 PRECURSOR, | | | | |
| | MICROSOMAL (UDPGT) | | | | |
| | (HYODEOXYCHOLIC | | | | |
| | ACID) (HLUG25) | | | | |
| | (UDPGTH-1) | | | | |
| gi\|484383\|pir\|\|JN0619 | glucuronosyltransferase | 528 | 354/536 | 408/536 | 0.0 |
| | (EC 2.4.1.17) 2B-4 | | (66%) | (76%) | |
| | precursor - human | | | | |
| gi\|3153832\|gb\|AAC95002.1\| | UDP- | 528 | 354/536 | 409/536 | 0.0 |
| (AF064200) | glucuronosyltransferase | | (66%) | (76%) | |
| | 2B4 precursor [Homo | | | | |
| | sapiens] | | | | |
| gi\|4079707\|gb\|AAC98726.1\| | UDP- | 529 | 351/536 | 410/536 | 0.0 |
| (AF016310) | glucuronosyltransferase | | (65%), | (76%) | |
| | [Macaca fascicularis] | | | | |

The homology between these and other sequences is shown graphically in the ClustalW analysis shown in Table 40D. In the ClustalW alignment of the NOV40 protein, as well as all other ClustalW analyses herein, the black outlined amino acid residues indicate regions of conserved sequence (i.e., regions that may be required to preserve structural or functional properties), whereas non-highlighted amino acid residues are less conserved and can potentially be altered to a much broader extent without altering protein structure or function.

[image]

[image]

Table 40E lists the domain descriptions from DOMAIN analysis results against NOV40. This indicates that the NOV40 sequence has properties similar to those of other proteins known to contain this domain.

TABLE 40E
Domain Analysis of NOV40

gnL|Pfam|pfam00201, UDPGT, UDP-glucoronosyl and UDP-glucosyl
transferase. (SEQ ID NO:835)
CD-Length = 501 residues, 100.0% aligned
Score = 587 bits (1514) , Expect = 4e−169

| | | | |
|---|---|---|---|
| NOV40: | 24 | GNVPLWPMEYSPWMNIKTILDKLMQISHEVTVLTLSASILVDPNITSVTKFEVYSISVIK | 83 |
| | | \| \| +\|\|\|+ \| \|\|\|+\| \|\| +\|+\| \|\|\|\|\|\| \|\|\|\|\|+ \| \| \|\|\| \| \| \| | |
| Sbjct: | 1 | GKVLVWPMDGSHWMNMKGILLELVQRGHEVTVLRPSASILIGPAKPSNLKFETYPDSATK | 60 |
| NOV40: | 84 | DDFAGFFFTQQITKWIHDLPKHIFWFKCVPFKNILW-------EYSGYTEKFFKDVVLNK | 136 |
| | | ++ \| \| + + \|\| +\| \|\|\| \|++\| \|\| | |
| Sbjct: | 61 | EELENLF-----PKRVMN------WFMEAAEAGTVWSYFSALQEYSDGARVSCKELVGNK | 109 |
| NOV40: | 137 | KLMTNLQESRSDVVHANAIGPFGELLAELLKISFVYSLHFSPGYTFEKYSGGFLLPPSYG | 196 |
| | | \|\|\| \|\|\|\| \|\|\| \|+ + \| \| \|\|\|\|\|\| \| \|\|\|\| \| \|\|\| \|\| \|\| \|\|\|\| | |
| Sbjct: | 110 | FLMTKLQESSFDVVLADPVWPCGALLAELLHIPTVYSLRFVPGYAAEKADGGLPAPPSYV | 169 |
| NOV40: | 197 | AVILSELSGSMTFMETVRNIIYVFYFDFWFQTFDMKKGDQFYSEVLGKSCFLSEIMGKAE | 256 |
| | | \| \|\|+\|\| \|\|\| \| \|+\|++ + \|\|\|\|\|\|\| \| \|\| \|\|\| \|\|+\|\|+ \| \| + \|\| | |
| Sbjct: | 170 | PVRLSDLSDCMTFGERVKNMLIMLYFDFWFQRFP-KKWDQFASELLGRPVTLPEDLSYAS | 228 |
| NOV40: | 257 | MWLIRNYWYLEFPRPLLPNFEFVVRLYCKPVNPLPKEKMEEFAQSSDEDGVV-FSLESAV | 315 |
| | | \|\|+\|\|\|\| \|\|\|\|\|\|\|\|\|\| \|\|+ \| \|\|\| \|\|\|+\| \|\| \| \|\|\| \| \|\|\| \|\|\| \| \| | |
| Sbjct: | 229 | AWLLRNYWDLEFPRPLLPNMEFIGGLNCKPAKPLPQE-MEAFVQSSGEHGWJVFSLGSMV | 287 |
| NOV40: | 316 | QNLTEEKADLITSALAQIPQKVM-KF-GRKPNTLRSNTQWHRWIPQNECLILDHPQTKQF | 373 |
| | | \|+ \|\|\|\|+ \| \|\|\|\|\|\|\|\|\|\|+ +\| \| \|\|+\|\| +\|\|+ +\|+\|\|\|+ +\| \|\|+\|+\|\| | |
| Sbjct: | 288 | SNIPEEKANEIASALAQIPQKVLWRFDCTKPSTLGNNTRLVKWLPQND--LLGHPKTRAF | 345 |
| NOV40: | 374 | ITYGGTNSIYEMIYRGVPSMGIPLFADQHDNIAHMKAKGAAVILDLSTKSSTDLLDISVF | 433 |
| | | +\|+ \|+\| +\|\| \| \|\|\| +\|+\|\|\| \|\| \|\| \|\|+\|\|\|\|\|\| \|++ \| +\| \|\|\|+ | |
| Sbjct: | 346 | VTHAGSNGVYEAICHGVPMVGMPLFGDQMDNAKHMEAKGAAVTLNVLTMTSEDLLNALK- | 404 |
| NOV40: | 434 | VSLFLSFRYKESVMKLSRIQHDQPVKPLDRAVFWIEFVMRHKGAKHLRVAARDLTWFQYH | 493 |
| | | ++ \|\|\|++\|+\|\| \| \|\|\|\|\|\|\|\|\|\|\|\|\|\|\|\|\|\|\|\|\|\|\|\|\|\|\|\| \|\| \|\|\|\|+\|\|\| | |
| Sbjct: | 405 | -TVINDPSYKENIMRLSSIHHDQPVKPLDRAVFWIEFVMRHKGAKHLRPAAHDLTWYQYH | 463 |
| NOV40: | 494 | SLDVIGFLLACVATVTFIITKCCLFCFWKFTRKVKKEK 531 | |
| | | \|\|\|\|\|\|\|\|\|\|\|\|\|\| \|\| \|\|\|\|\| + \|\| \| \|+ \| | |
| Sbjct: | 464 | SLDVIGFLLACVATVAFITFKCCLFGYRKFVGKKKRVK 501 | |

The UDP-glucuronosyltransferases, a group of isoenzymes located primarily in hepatic endoplasmic reticulum and nuclear envelope, are encoded by a large multigene family that has evolved to produce catalysts with differing but overlapping substrate specificities. Two subfamilies are recognized by sequence identities. UGT1 consists of at least 4 isoenzymes that catalyze the glucuronidation of phenols and bilirubin. All 4 map to chromosome 2 and probably derive from the same gene (UGT1). The UGT2 family contains at least 5 members catalyzing steroid or bile acid glucuronidation. Members of the subfamily share 65 to 90% amino acid sequence identity. However, unlike the phenol UGT cDNAs, where the high degree of identity is concentrated in the 3-prime region of the cDNA, the steroid UGTs have a high degree of sequence homology throughout the cDNA. The disclosed NOV40 nucleic acid of the invention encoding a UDP Glycosyltransferase-like protein includes the nucleic acid whose sequence is provided in Table 40A or a fragment thereof. The invention also includes a mutant or variant nucleic acid any of whose bases may be changed from the corresponding base shown in Table 40A while still encoding a protein that maintains its UDP[Glycosyltransferase-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 19 percent of the bases may be so changed.

The disclosed NOV40 protein of the invention includes the UDP Glycosyltransferase-like protein whose sequence is provided in Table 40B. The invention also includes a mutant or variant protein any of whose residues may be changed from the corresponding residue shown in Table 40B while still encoding a protein that maintains its UDP Glycosyltransferase-like activities and physiological functions, or a functional fragment thereof. In the mutant or variant protein, up to about 35 percent of the residues may be so changed.

The invention further encompasses antibodies and antibody fragments, such as F_{ab} or (F_{ab})₂, that bind immunospecifically to any of the proteins of the invention.

The above disclosed information suggests that this UDP Glycosyltransferase-like protein (NOV40) is a member of a “UDP Glycosyltransferase family”. Therefore, the NOV40 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV40 nucleic acids and proteins of the invention are useful in potential therapeutic applications implicated in brain disorders including Crigler-Najjar syndrome, Gilbert syndrome, and/or other diseases and pathologies.

NOV40 nucleic acids and polypeptides are further useful in the generation of antibodies that bind immuno-specifically to the novel NOV40 substances for use in therapeutic or diagnostic methods. These antibodies may be generated according to methods known in the art, using prediction from hydrophobicity charts, as described in the “Anti-NOVX Antibodies” section below. The disclosed NOV40 protein has multiple hydrophilic regions, each of which can be used as an immunogen. These novel proteins can be used in assay systems for functional analysis of various human disorders, which will help in understanding of pathology of the disease and development of new drug targets for various disorders.

NOV41

NOV41 includes three novel adrenal secretory serine protease-like proteins disclosed below. The disclosed sequences have been named NOV41a and NOV41b.

NOV41a

A disclosed NOV41a nucleic acid of 2155 nucleotides (also referred to as CG56680-01) encoding a Sodium/Hydrogen Exchanger 4-like protein is shown in Table 41A. An open reading frame was identified beginning with a ATG initiation codon at nucleotides 16-18 and ending with a TAG codon at nucleotides 2140-2142. The start and stop codons are shown in bold in Table 41A, and the 5′ and 3′ untranslated regions, if any, are underlined.

TABLE 41A
NOV41a nucleotide sequence.

(SEQ ID NO:153)

GAGAAGCCCACAGGAATGGCTCTGCAGATGTTCGTGACTTACAGTCCTTGGAATTGTTTGCTACTGCTAGTG
GCTCTTGAGTGTTCTGAAGCATCTTCTGATTTGAATGAATCTGCAAATTCCACTGCTCAGTATGCATCTAAC
GCTTGGTTTGCTGCTGCCAGCTCAGAGCCAGAGGAAGGGATATCTGTTTTTGAACTGGATTATGACTATGTG
CAAATTCCTTATGAGGTCACTCTCTGGATACTTCTAGCATCCCTTGCAAAAATAGGTTTCCACCTCTACCAC
AGGCTGCCAGGCCTCATGCCAGAAAGCTGCCTCCTCATCCTGGTGGGGGCCCTGGTGGGCGGCATCATCTTC
CGCACCCACCACAAATCGCCTCCGGTCATGGACTCCAGCATCTACTTCCTGTATCTCCTGCCACCCATCGTT
CTGGAGGGCGGCTACTTCATGCCCACCCGGCCCTTCTTTGAGAACATCGGCTCCATCCTGTGGTGGGCAGTA
TTGGGGCCCCTGATCAACCACCTTGGGCATTGGCCTCTCCCTCTACCTCATCTGCCAGGTGAAGGCCTTTGGC
CTGGGCGACGTCAACCTGCTGCAGAACCTGCTGTTCGGCAGCCTGATCTCCGCCGTGCACCCAGTGCCCGTG
CTAGCCGTGTTTGAGGAAGCGCGCGTGAACGAGCAGCTCTACATGATGATCTTTGGGGAGCCCCTCCTCAAT
GATGGCATTACTGTGGTGGTCTTATACAATATGTTAATTGCCTTTACAAAGATGCATAAATTTGAAGACATA
GAAACTGTCGACATTTTGGCTGGATGTGCCCGATTCATCGTTGTGGGGCTTGGAGCGGTATTGTTTGGCATC
GTTTTTGGATTTATTTCTGCATTTATCACACGTTTCACTCAGAATATCTCTGCAATTGAGCCACTCATCGTC
TTCATGTTCAGCTATTTGTCTTACTTAGCTGCTGAAACCCTCTATCTCTCCGGCATCCTGGCGATCACAGCC
TGCGCAGTAACAATGAAAAAGTACGTGGAAGAAAACGTGTCCCAGACATCATACACGACCATCAAGTACTTC
ATGAAGATGCTGAGCAGCGTCAGCGAGACCTTGATCTTCATCTTCATGGGTGTGTCCACTGTGGGCAAGAAT
CACGAGTGGAACTGCGCCTTCATCTGCTTCACCCTGGCCTTCTGCCAAATCTGGAGAGCCATCAGTGTATTT
GCTCTCTTCTATATCAGTAACCAGTTTTCGGACTTTCCCCTTCTCCATCAAGGACCAGTGCATCATTTTCTAC
AGTGGTGTTCGAGGAGCTCGAAGTTTTTCACTTGCATTTTTGCTTCCTCTGTCTCTTTTTCCTAGGAAGAAA
ATGTTTGTCACTGCTACTCTAGTAGTTATATACTTTACTGTATTTATTCAGGGAATCACAGTTGGCCCTCTG
GTCAGGTACCTGGATGTTAAAAAAACCAATAAAAAAGAATCCATCAATGAAGAGCTTCATATTCGTCTGATG
GATCACTTAAAGGCTGGAATCGAAGATGTGTGTGGGCACTGGAGTCACTACCAAGTGAGAGACAAGTTTAAG
AAGTTTGATCATAGATACTTACGGAAAATCCTCATCAGAAAGAACCTACCCAAATCAAGCATTGTTTCTTTG
TACAAGAAGCTGGAAATGAAGCAAGCCATCGAGATGGTGGAGACTGGGATACTGAGCTCTACAGCTTTCTCC
ATACCCCATCAGGCCCAGAGGATACAAGGAATCAAAAGACTTTCCCCTGAAGATGTGGAGTCCATAAGGGAC
ATTCTGACATCCAACATGTACCAAGTTCGGCAAAGGACCCTGTCCTACAACAAATACAACCTCAAACCCCAA
ACAGTGAGAACCAGGCTAAAGAGATTCTGATCCGCCGCCAGAACACCTTAAGGGAGAGCATGAGGAAAGGT
CACAGCCTGCCCTGGGGAAAGCCGGCTGGCACCAAGAATATCCGCTACCTCTCCTACCCCTACGGGAATCCT
CAGTCTGCAGGAAGAGACACAAGGGCTGCTGGGTTCTCAGGTAAGCTGCCCACCTGGCTGCTCTGCTGCTTT
TCTGTAGAGTCAGGTGGTAAATATCTGGGGGTGTGGGCCAAGAGGCAACATTAAGAACATTATGTAG

In a search of public sequence databases, the NOV41a nucleic acid sequence, located on chromosome 2, has 1820 of 2156 bases (84%) identical to a gb:GENBANK-ID:RATNHEXIV|acc:M85301.1 mRNA from Rattus norvegicus (Rat sodium-hydrogen exchange protein-isoform 4 (NHE-4) mRNA, complete cds) (E=6.4e⁻²¹⁷).

The disclosed NOV41a polypeptide (SEQ ID NO:154) encoded by SEQ ID NO:153 has 708 amino acid residues and is presented in Table 41B using the one-letter amino acid code. Signal P, Psort and/or Hydropathy results predict that NOV41a has a signal peptide and is likely to be localized to the plasma membrane with a certainty of 0.8200. Alternatively, NOV41a may also localize to the Golgi body with a certainty of 0.4600, to the endoplasmic reticulum (membrane) with a certainty of 0.3700, or to the endoplasmic reticulum (lumen) with a certainty of 0.1000. The most likely cleavage site for NOV41a is between positions 26 and 27: SEA-SS.

TABLE 41B
Encoded NOV41a protein sequence.

(SEQ ID NO:154)

MALQMFVTYSPWNCLLLLVALECSEASSDLNESANSTAQYASNAWFAAASSEPEEGISVFELDYDYVQIPYE
VTLWILLASLAKIGFHLYHRLPGLMPESCLLILVGALVGGIIFGTDHKSPPVMDSSTYFLYLLPPIVLEGGY
FMPTRPFFENICSILWWAVLGALINALGTGLSLYLICQVKAFGLGDVNLLQNLLFCSLISAVDPVAVLAVFE
EARVNEQLYMMIFGEALLNDGITVVVLYNMLIAFTKMHKFEDIETVDILAGCARFIVVGLGGVLFGIVFGFI
SAFITRFTQNISAIEPLIVFMFSYLSYLAAETLYLSGILAITACAVTMKKYVEENVSQTSYTTIKYFMKMLS
SVSETLIFIFMGVSTVGKNHEWNWAFICFTLAFCQIWRAISVFALFYISNQFRTFPFSIKDQCIIFYSGVRG
AGSFSLAFLLPLSLFPRKKMFVTATLVVIYFTVFIQGITVGPLVRYLDVKKTNKKESINEELHIRLMDHLKA
GIEDVCGHWSHYQVRDKFKKFDHRYLRKILIRKNLPKSSIVSLYKKLEMKQAIEMVETGILSSTAFSIPHQA
QRIQGIKRLSPEDVESIRDILTSNMYQVRQRTLSYNKYNLKPQTSEKQAKEILIRRQNTLRESMRKGHSLPW
GKPAGTKNIRYLSYPYGNPQSAGRDTRAAGFSGKLPTWLLCCFSVESGGKYLGVWAKRQH

A search of sequence databases reveals that the NOV41a amino acid sequence has 599 of 688 amino acid residues (87%) identical to, and 631 of 688 amino acid residues (91%) similar to, the 717 amino acid residue ptnr:SWISSPROT-ACC:P26434 protein from Rattus norvegicus (Rat) (Sodium/Hydrogen Exchanger 4 (NA(+)/H(+) Exchanger 4) (NHE-4)) (E=0.0).

NOV41a is predicted to be expressed in at least the stomach. This information was derived by determining the tissue sources of the sequences that were included in the invention including but not limited to SeqCalling sources, Public EST sources, Literature sources, and/or RACE sources.

In addition, the sequence is predicted to be expressed in stomach, colon and small intestine; lesser amounts in kidney, brain, uterus and skeletal muscle because of the expression pattern of (GENBANK-ID: gb:GENBANK-ID:RATNHEXIV|acc:M85301.1) a closely related Rat sodium-hydrogen exchange protein-isoform 4 (NHE-4) mRNA, complete cds homolog.

NOV41b

In the present invention, the target sequence identified previously, NOV41a, was subjected to the exon linking process to confirm the sequence. PCR primers were designed by starting at the most upstream sequence available, for the forward primer, and at the most downstream sequence available for the reverse primer. In each case, the sequence was examined, walking inward from the respective termini toward the coding sequence, until a suitable sequence that is either unique or highly selective was encountered, or, in the case of the reverse primer, until the stop codon was reached. Such primers were designed based on in silico predictions for the full length cDNA, part (one or more exons) of the DNA or protein sequence of the target sequence, or by translated homology of the predicted exons to closely related human sequences sequences from other species. These primers were then employed in PCR amplification based on the following pool of human cDNAs: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea, uterus. Usually the resulting amplicons were gel purified, cloned and sequenced to high redundancy. The resulting sequences from all clones were assembled with themselves, with other fragments in CuraGen Corporation's database and with public ESTs. Fragments and ESTs were included as components for an assembly when the extent of their identity with another component of the assembly was at least 95% over 50 bp. In addition, sequence traces were evaluated manually and edited for corrections if appropriate. These procedures provide the sequence reported below, which is designated NOV41b. This differs from the previously identified sequence (NOV41a) in having 17 different aminoacids.

A disclosed NOV41b nucleicacid of 2436 nucleotides (also referred to as CG56680-02) encoding a Sodium/Hydrogen Exchanger 4-like protein is shown in Table 41C. An open reading frame was identified beginning with a ATG initiation codon at nucleotides 86-88 and ending with a TAA codon at nucleotides 2369-2371. The start and stop codons are shown in bold in Table 41C, and the 5′ and 3′ untranslated regions, if any, are underlined.

TABLE 41C
NOV41b nucleotide sequence.

(SEQ ID NO:155)

ATGCAGTCACTCTCTAGAAGCCTCCCCGACTTCAGATGTGTGGCACACATCCACACAGGGGTGTAGGTAGGA
GAAGCCCACAGGAATGGCTCTGCAGATGTTCGTGACTTACAGTCCTTGGAATTGTTTGCTACTGCTAGTGGC
TCTTGAGTGTTCTGAAGCATCTTCTGATTTGAATGAATCTGCAAATTCCACTGCTCAGTATGCATCTAACGC
TTGGTTTGCTGCTGCCAGCTCAGAGCCAGAGGAAGGGATATCTGTTTTTGAACTGCATTATGACTATGTGCA
AATTCCTTATGAGGTCACTCTCTGGATACTTCTAGCATCCCTTGCAAAAATAGGCTTCCACCTCTACCACAG
GCTGCCAGGCCTCATGCCAGAAAGCTGCCTCCTCATCCTGCTGGGGGCGCTGGTGGGCGGCATCATCTTCGG
CACCGACCACAAATCGCCTCCGGTCATGGACTCCAGCATCTACTTCCTGTATCTCCTGCCACCCATCGTTCT
GGAGGGCAACTACTTCATGCCCACCCGGCCCTTCTTTGAGAACATCGGCTCCATCCTCTGCTAAGCAGTATT
GGGGGCCCTGATCAACGCCTTGGGCATTGGCCTCTCCCTCTACCTCATCTGCCAGGTGAAGGCCTTTGGCCT
GGGCGACGTCAACCTGCTGCAGAACCTGCTGTTCGGCAGCCTGATCTCCGCCGTGGACCCAGTGGCCGTGCT
AGCCGTGTTTGAGGAACCGCGCGTCAACGAGCAGCTCTACATGATGATCTTTGGGGAGGCCCTGCTCAATGA
TGGCATTACTGTGGTGTTATACAATATGTTAATTGCCTTTACAAAGATGCATAAATTTGAAGACATAGAAAC
TGTCGACATTTTGGCTGGATGTGCCCGATTCATCCTTGTGGGGCTTGGAGGGGTATTGTTTGGCATCGTTTT
TGGATTTATTTCTGCATTTATCACACGTTTCACTCAGAATATCTCTGCAATTGAGCCACTCATCGTCTTCAT
GTTCAGCTATTTGTCTTACTTAGCTGCTGAAACCCTCTATCTCTCCGGCATCCTGGCGATCACAGCCTGCGC
AGTAACAATGAAAAAGTACGTGGAAGAAAACGTGTCCCAGACATCATACACGACCATCAAGTACTTCATGAA
GATGCTGAGCAGCGTCAGCGAGACCTTGATCTTCATCTTCATGGGTGTGTCCACTGTGGGCAAGAATCACGA
GTGGAACTGGGCCTTCATCTGCTTCACCCTGGCCTTCTGCCAAATCTGGAGAGCCATCAGTGTATTTGCTCT
CTTCTATATCAGTAACCAGTTTCGGACTTTCCCCTTCTCCATCAAGGACCAGTGCATCATTTTCTACAGTGG
TGTTCCAGGAGCTGGAAGTTTTTCACTTGCATTTTTGCTTCCTCTGTCTCTTTTTCCTAGGAAGAAAATGTT
TGTCACTGCTACTCTAGTAGTTATATACTTTACTGTATTTATTCAGGGAATCACAGTTGGCCCTCTGGTCAG
GTACCTGGATGTTAAAAAAACCAATAAAAAAGAATCCATCAATGAAGAGCTTCATATTCGTCTGATGGATCA
CTTAAAGGCTGGAATCGAAGATGTGTGTGGGCACTGGAGTCAACTACCAAGTAGAGACAAGTTTAAGAAGTT
TGATCATAGATACTTACGGAAAATCCTCATCAGAAAGAACCTACCCAAATCAAGCATTCTTTCTTTGTACAA
GAAGCTGGAAATGAAGCAAGCCATCGAGATGGTGGAGACTGGGATACTGAGCTCTACAGCTTTCTCCATACC
CCATCAGGCCCAGAGGATACAAGGAATCAAAAGACTTTCCCCTGAAGATGTGGAGTCCATAAGGGACATTCT
GACATCCAACATGTACCAAGTTCGGCAAAGGACCCTGTCCTACAACAAATACAACCTCAAACCCCAAACAAG
TGAGAAGCAGGCTAAAGAGATTCTGATCCGCCGCCAGAACACCTTAAGGGAGAGCATCAGGAAAGGTCACAG
CCTGCCCTGGGGAAAGCCGGCTGCCACCAAGAATATCCGCTACCTCTCCTACCCCTACGGGAATCCTCAGTC
TGCAGGAAGACACACAAGGGCTGCTGGGTTCTCAGGTAAGCTGCCCACCTCGCTGCTCCTTTGGTTGAGGTT
CGGTCGAGGTGGACAGCTGACCATGGACACAACAGGGACCATCACAGGTCCCATAGTCCTTTGCTCCAAAAA
AATAGTGTTATTGTCCACAAGATTGTTTTGGTGTTTCTCAAGAGTCTGTCTTCCTATTAACTGTGAAAGGAG
GATTTCTGGAATTCAGAAGAGAGCTATTGAGTTTGCTGTGTTCAAGCTATTAAACATGGATCTATAAGCAGC
AGAGATTTTTTCCAACGACTGGGAGCAAACTTGCAGGCTCTGCCATGTACTTATTGTG

In a search of public sequence databases, the NOV41b nucleic acid sequence, located on chromosome 2, has 1818 of 2163 bases (84%) identical to a gb:GENBANK-ID:RATNHEXIV|acc:M85301.1 mRNA from Rattus norvegicus (Rat sodium-hydrogen exchange protein-isoform 4 (NHE-4) mRNA, complete cds) (E=0.0).

The disclosed NOV41b polypeptide (SEQ ID NO:156) encoded by SEQ ID NO:155 has 761 amino acid residues and is presented in Table 41D using the one-letter amino acid code. Signal P, Psort and/or Hydropathy results predict that NOV41b has no signal peptide and is likely to be localized to the plasma membrane with a certainty of 0.8200. Alternatively, NOV41b may also localize to the Golgi body with a certainty of 0.4600, to the endoplasmic reticulum (membrane) with a certainty of 0.3700, or to the endoplasmic reticulum (lumen) with a certainty of 0.1000. The most likely cleavage site for NOV41b is between positions 26 and 27: SEA-SS.

TABLE 41D
Encoded NOV41b protein sequence.

(SEQ ID NO:156)

MALQMFVTYSPWNCLLLLVALECSEASSDLNESANSTAQYASNAWFAAASSEPEEGISVFELDYDYVQIPYE
VTLWILLASLAKIGFHLYHRLPGLMPESCLLILVGALVGGIIFGTDHKSPPVMDSSIYFLYLLPPIVLEGGY
FMPTRPFFENIGSILWWAVLGALINALGIGLSLYLICQVKAFGLGDVNLLQNLLFGSLISAVDPVAVLAVFE
EARVNEQLYMMIFGEALLNDGITVVLYNMLIAFTKMHKFEDIETVDILAGCARFIVVGLGGVLFGIVFGFIS
AFITRFTQNISAIEPLIVEMFSYLSYLAAETLYLSGILAITACAVTMKKYVEENVSQTSYTTIKYFMKMLSS
VSETLIFIFMGVSTVGKNHEWNWAFICFTLAFCQIWRAISVFALFYISNQFRTFPFSIKDQCIIFYSGVRGA
GSFSLAFLLPLSLFPRKKMFVTATLVVIYFTVFIQGITVGPLVRYLDVKKTNKKESINEELHIRLMDHLKAG
IEDVCGHWSHYQVRDKFKKFDHRYLRKILIRKNLPKSSIVSLYKKLEMKQAIEMVETGILSSTAFSIPHQAQ
RIQGIKRLSPEDVESIRDILTSNMYQVRQRTLSYNKYNLKPQTSEKQAKEILIRRQNTLRESMRKGHSLPWG
KPAGTKNIRYLSYPYGNPQSAGRDTRAAGFSGKLPTWLLLWLRFGRGGQLTMDTAGTITGPIVLCSKRNSVI
VHKIVLVFLKSLSSYNCERRISCIQKRAIEFAVLKLLNMDL

A search of sequence databases reveals that the NOV41b amino acid sequence has 606 of 717 amino acid residues (84%) identical to, and 641 of 717 amino acid residues (89%) similar to, the 717 amino acid residue ptnr:SWISSPROT-ACC:P26434 protein from Rattus norvegicus (Rat) (Sodium/Hydrogen Exchanger 4 (NA(+)/H(+) Exchanger 4) (NHE-4)) (E=0.0).

NOV41b is predicted to be expressed in at least adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea and uterus.

NOV41a also has homology to the amino acid sequences shown in the BLASTP data listed in Table 41E.

TABLE 41E
BLAST results for NOV41a

| Gene Index/ | | Length | Identity | Positives | |
|---|---|---|---|---|---|
| Identifier | Protein/Organism | (aa) | (%) | (%) | Expect |
| gi\|127814\|sp\|P26434\| | SODIUM/HYDROGEN | 717 | 599/688 | 631/688 | 0.0 |
| NAH4_RAT | EXCHANGER 4 | | (87%) | (91%) | |
| | (NA(+)/H(+) | | | | |
| | EXCHANGER 4) | | | | |
| | (NHE-4) | | | | |
| gi\|1346658\|sp\|P48763\| | SODIUM/HYDROGEN | 813 | 421/659 | 523/659 | 0.0 |
| NAH2_RAT | EXCHANGER 2 | | (63%) | (78%) | |
| | (NA(+)/H(+) | | | | |
| | EXCHANGER 2) | | | | |
| | (NHE-2) (H7) | | | | |
| gi\|1709222\|sp\|P50482\| | SODIUM/HYDROGEN | 809 | 419/659 | 522/659 | 0.0 |
| NAH2_RABIT | EXCHANGER 2 | | (63%) | (78%) | |
| | (NA(+)/H(+) | | | | |
| | EXCHANGER 2) | | | | |
| | (NHE-2) | | | | |
| gi\|15529998\|ref\|NP_{—} | solute carrier | 812 | 405/611 | 499/611 | 0.0 |
| 003039.2\| | family 9 | | (66%) | (81%) | |
| (NM_003048) | (sodium/hydrogen | | | | |
| | exchanger), | | | | |
| | isoform 2 [Homo | | | | |
| | sapiens] | | | | |
| gi\|6981560\|ref\|NP_036785.1\| | solute carrier | 697 | 372/560 | 457/560 | 0.0 |
| (NM_012653) | family 9 | | (66%) | (81%) | |
| | (sodium/hydrogen | | | | |
| | exchanger 2), | | | | |
| | antiporter 2, | | | | |
| | Na+/H+ (Na+/H+ | | | | |
| | exchanger 2) | | | | |
| | [Rattus | | | | |
| | norvegicus] | | | | |

The homology between these and other sequences is shown graphically in the ClustalW analysis shown in Table 41F. In the ClustalW alignment of the NOV41 protein, as well as all other ClustalW analyses herein, the black outlined amino acid residues indicate regions of conserved sequence (i.e., regions that may be required to preserve structural or functional properties), whereas non-highlighted amino acid residues are less conserved and can potentially be altered to a much broader extent without altering protein structure or function.

[image]

[image]

[image]

Na+/H+ antiporters are key transporters in maintaining the pH of actively metobolizing cells. Na+/H+ exchange proteins eject protons from cells, effectively eliminating excess acid from actively metabolising cells. Na+/H+ exchange activity is also crucial for the regulation of cell volume, and for the reabsorption of NaCl across renal, intestinal, and other epithelia. These antiports exchange Na+ for H+ in an electroneutral manner, and this activity is carried out by a family of Na+/H+ exchangers, or NHEs. In mammalian cells, Na+/H+ exchange activity is found in both the plasma membrane and inner mitochondrial membrane. To date, six mammalian isoforms have been identified (designated NHE1-NHE6). These exchangers are highly-regulated (glyco)phosphoproteins, which, based on their primary structure, appear to contain 10-12 transmembrane regions at the N-terminus and a large cytoplasmic region at the C-terminus. The transmembrane regions M3-M12 share identity with other members of the family. The M6 and M7 regions are highly conserved. Thus, this is thought to be the region that is involved in the transport of sodium and hydrogen ions. The cytoplasmic region has little similarity throughout the family. There is some evidence that they may exist in the cell membrane as homodimers, but the molecular mechanisms of antiport are unclear. Na+/H+ antiporters play an important role in signal transduction.

The disclosed NOV41 nucleic acid of the invention encoding a Sodium/Hydrogen Exchanger 4-like protein includes the nucleic acid whose sequence is provided in Table 41A, 41C or a fragment thereof. The invention also includes a mutant or variant nucleic acid any of whose bases may be changed from the corresponding base shown in Table 41A or 41C while still encoding a protein that maintains its UDP[Glycosyltransferase-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 16 percent of the bases may be so changed.

The disclosed NOV41 protein of the invention includes the Sodium/Hydrogen Exchanger 4-like protein whose sequence is provided in Table 41B or 41D. The invention also includes a mutant or variant protein any of whose residues may be changed from the corresponding residue shown in Table 41B or 41D while still encoding a protein that maintains its Sodium/Hydrogen Exchanger 4-like activities and physiological functions, or a functional fragment thereof. In the mutant or variant protein, up to about 37 percent of the residues may be so changed.

The invention further encompasses antibodies and antibody fragments, such as F_{ab} or (F_{ab})₂, that bind immunospecifically to any of the proteins of the invention.

The above disclosed information suggests that this Sodium/Hydrogen Exchanger 4-like protein (NOV41) is a member of a “Sodium/Hydrogen Exchanger 4 family”. Therefore, the NOV41 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV41 nucleic acids and proteins of the invention are useful in potential therapeutic applications implicated in brain disorders including hypercalceimia, ulcers, inflammatory bowel disease, diverticular disease; diseases of the kidney including diabetes, autoimmune disease, renal artery stenosis, interstitial nephritis, and others; diseases of the brain including Von Hippel-Lindau (VHL) syndrome, Alzheimer's disease, epilepsy, and others; endometriosis, fertility, muscular dystrophy, Lesch-Nyhan syndrome, myasthenia gravis, and/or other diseases and pathologies.

NOV41 nucleic acids and polypeptides are further useful in the generation of antibodies that bind immuno-specifically to the novel NOV41 substances for use in therapeutic or diagnostic methods. These antibodies may be generated according to methods known in the art, using prediction from hydrophobicity charts, as described in the “Anti-NOVX Antibodies” section below. The disclosed NOV41 protein has multiple hydrophilic regions, each of which can be used as an immunogen. These novel proteins can be used in assay systems for functional analysis of various human disorders, which will help in understanding of pathology of the disease and development of new drug targets for various disorders.

NOV42

NOV42 includes three novel Kupffer Cell Receptor-like proteins disclosed below. The disclosed sequences have been named NOV42a, NOV42b, NOV42c, and NOV42d.

NOV42a

A disclosed NOV42a nucleic acid of 1760 nucleotides (also referred to as CG56682-01) encoding a Kupffer Cell Receptor-like protein is shown in Table 42A. An open reading frame was identified beginning with a ATG initiation codon at nucleotides 16-18 and ending with a TGA codon at nucleotides 1661-1663. The start and stop codons are shown in bold in Table 42A, and the 5′ and 3′ untranslated regions, if any, are underlined.

TABLE 42A
NOV42a nucleotide sequence.

(SEQ ID NO:157)

TGGCTGGGAGCAGTGCTGGAGGATGAAGGAAGCAGAGATGGACGGTGAGGCAGTCCGCTTCTGCACAGATAA
CCAGTGTGTCTCCCTGCACCCCCAGGTGTGGACTCTGTCGCAAATGGCTCCTGCAGCCCCCAAGATACCGAG
GCTCGTTCACGCTACCCCGGCATTTATGGCTGTGACCTTGGTCTTCTCTCTTGTGACTCTCTTTGTAGTGGG
TAAGCCCCCAGGTGACCCAAATCTCACTAACTTTCTCTCCTTTCAGCACAAAGTCCCCAGGGGCCCCAGATG
CACACTCGATCATCACCACTTTGGCAGGGAGGCAGAAATGCGAGAGCTTATCCAGACATTTAAAGGCCACAT
GGAGAATTCCAGTGCCTGGGTAGTAGAAATCCAGATGTTGAAGTGCAGAGTGGACAATGTCAATTCGCAGCT
CCAGGTGCTCGGTGATCATCTGGGAAACACCAATGCTGACATCCAGATGGTAAAAGGAGTTCTAAAGGATGC
CACTACATTGAGTTTGCAGACACAGATGTTAAGCAGTTCCCTGGAGGGAACCAATGCTGAGATCCAGAGGCT
CAAGGAAGACCTTGAAAAGGCAGATGCTTTAACTTTCCAGACGCTGAATTTCTTAAAAAGCAGTTTAGAAAA
CACCAGCATTGAGCTCCACGTGCTAAGCAGAGGCTTAGAAAATGCAACTCTGAAATTCAGATGTTGAATGC
CAGTTTGGAAACGGCAAATGCTTTAAACTCCCAGACCCAGGCCTTTATAAAAAGCAGTTTTGACAACACTAG
TGCTGAGATCCAGTTCTTAAGAGGTCATTTGGAAAGAGCTGGTGATGAAATTCACGTGTTAAAAAGGGATTT
GAAAATGGTCACAGCCCAGACCCAAAAAGCAAATGGCCGTCTCGACCAGACACATACTCAGATTCAGGTATT
CAAGTCAGAGATGGAAAATGTGAATACCTTAAATGCCCAGATTCAGGTCTTAAATGGTCATATGAAAAATGC
CAGCAGAGAGATACAGACCCTAAACAAGGAATGAAGAATGCTTCAGCCTTAAACTTCCCAGACCCAGATGTT
AGACAGCAATCTGCAGAAGGCCAGTGCCGAGATCCAGAGGTTAAGAGGGGATCTAGAGAACACCAAAGCTCT
AACCATGGAAATCCAGCAGGAGCAGAGTCGCCTGAAGACCCTCCATGTGGTCATTACTTCACAGGAACAGCT
ACAAAGAACCCAAAGTAAGCAGCTTCTCCAGATGGTCCTGCAAGGCTGGAAGTTCAATGGTGGAAGCTTATA
TTATTTTTCTAGTGTCAAGAAGTCTTGGCATGAGGCTGAGCAGTTCTGCGTGTCCCAGGGAGCCCATCTGGC
ATCTGTGGCCTCCAAGGAGGAGCAGGCATTTCTGGTAGAGTTCACAAGTAAAGTGTACTACTGGATCGGTCT
CACTGACAGGGGCACAGAGGGCTCCTGGCGCTGGACAGATGGGACACCATTCAACGCCGCCCAGAACAAAGG
GTTTTGGGAAAAGAATCAGTCTGACAACTGGCGGCACAAGAATGGGCAGACTGAAGACTGTGTCCAAATTCA
GCAGAAGTGGAATGACATGACCTGTGACACCCCCTATCAGTGGGTGTGCAAGAAGCCCATGGGCCAGGGTGT
GGCCTGAGGGCAGGCCAGAGCTGAGGGGCTCCTCCTGCTTGCCAATACTGACCCTCCTCCTCGATGCCTTCG
GAGCCTCTGAGCTCTGCTTGTTCTCTGGGACC

In a search of public sequence databases, the NOV42a nucleic acid sequence, located on chromosome 2, has 1214 of 1730 bases (70%) identical to a gb:GENBANK-ID:D88577|acc:D88577.1 mRNA from Mus musculus (mRNA for Kupffer cell receptor, complete cds) (E=3.9e⁻¹⁶²).

The disclosed NOV42a polypeptide (SEQ ID NO:158) encoded by SEQ ID NO:157 has 546 amino acid residues and is presented in Table 42B using the one-letter amino acid code. Signal P, Psort and/or Hydropathy results predict that NOV42a has a signal peptide and is likely to be localized to the plasma membrane with a certainty of 0.7900. Alternatively, NOV42a may also localize to the microbody (peroxisome) with a certainty of 0.3000, to the Golgi body with a certainty of 0.3000, or to the endoplasmic reticulum (membrane) with a certainty of 0.2000. The most likely cleavage site for NOV42a is between positions 65 and 66: VVG-KP.

TABLE 42B
Encoded NOV42A protein sequence.

(SEQ ID NO:158)

MKEAEMDGEAVRFCTDNQCVSLHPQGVDSVAMAPAAPKIPRLVQATPAFMAVTLVFSLVTLFVVGKPPGDPN
LTNFLSFQHKVPRGPRCTLDHHHFGREAEMRELIQTFKGHMENSSAWVVEIQMLKCRVDNVNSQLQVLGDHL
GNTNADIQMVKGVLKDATTLSLQTQMLRSSLEGTNAEIQRLKEDLEKADALTFQTLNFLKSSLENTSIELHV
LSRGLENANSEIQMLNASLETANALNSQTQAFIKSSFDNTSAEIQFLRGHLERAGDEIHVLKRDLKMVTAQT
QKANGRLDQTDTQIQVFKSEMENVNTLNAQIQVLNGHMKNASREIQTLKQCMKNASALTSQTQMLDSNLQKA
SAEIQRLRGDLENTKALTMEIQQEQSRLKTLHVVITSQEQLQRTQSKQLLQMVLQGWKFNGGSLYYFSSVKK
SWHEAEQFCVSQGAHLASVASKEEQAFLVEFTSKVYYWIGLTDRGTEGSWRWTDGTPFNAAQNKGFWEKNQS
DNWRHKNGQTEDCVQIQQKWNDMTCDTPYQWVCKKPMGQGVA

A search of sequence databases reveals that the NOV42a amino acid sequence has 301 of 546 amino acid residues (55%) identical to, and 396 of 546 amino acid residues (72%) similar to, the 548 amino acid residue ptnr:SWISSPROT-ACC:P70194 protein from Mus musculus (Mouse) (Kupffer Cell Receptor) (E=0.0).

NOV42a is predicted to be expressed in at least cartilage. This information was derived by determining the tissue sources of the sequences that were included in the invention including but not limited to SeqCalling sources, Public EST sources, Literature sources, and/or RACE sources.

In addition, the sequence is predicted to be expressed in: Kupffer cells (liver) because of the expression pattern of(GENBANK-ID: gb:GENBANK-ID:D88577|acc:D88577.1) a closely related Mus musculus mRNA for Kupffer cell receptor, complete cds homolog.

NOV42b

In the present invention, the target sequence identified previously, NOV42a, was subjected to the exon linking process to confirm the sequence. PCR primers were designed by starting at the most upstream sequence available, for the forward primer, and at the most downstream sequence available for the reverse primer. In each case, the sequence was examined, walking inward from the respective termini toward the coding sequence, until a suitable sequence that is either unique or highly selective was encountered, or, in the case of the reverse primer, until the stop codon was reached. Such primers were designed based on in silico predictions for the full length cDNA, part (one or more exons) of the DNA or protein sequence of the target sequence, or by translated homology of the predicted exons to closely related human sequences sequences from other species. These primers were then employed in PCR amplification based on the following pool of human cDNAs: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea, uterus. Usually the resulting amplicons were gel purified, cloned and sequenced to high redundancy. The resulting sequences from all clones were assembled with themselves, with other fragments in CuraGen Corporation's database and with public ESTs. Fragments and ESTs were included as components for an assembly when the extent of their identity with another component of the assembly was at least 95% over 50 bp. In addition, sequence traces were evaluated manually and edited for corrections if appropriate. These procedures provide the sequence reported below, which is designated NOV42b. This differs from the previously identified sequence (NOV42a) in having 3 less aminoacids and 26 different ones.

A disclosed NOV42b nucleic acid of 1769 nucleotides (also referred to as CG56682-02) encoding a Kupffer cell receptor-like protein is shown in Table 42C. An open reading frame was identified beginning with a ATG initiation codon at nucleotides 23-25 and ending with a TGA codon at nucleotides 1670-1672. The start and stop codons are shown in bold in

TABLE 42C
NOV42b nucleotide sequence.

(SEQ ID NO:159)

TGGCTGGGAGCAGTGCTGGAGGATGAGGAAGCAGAGATGGACGGTGAGGCAGTCCGCTTCTGCACAGATAA
CCAGTGTGTCTCCCTGCACCCCCAAGAGGTGGACTCTGTGGCAATGGCTCCTGCAGCCCCCAAGATACCGAG
GCTCGTTCAGGCTACCCCGGCATTTATGGCTGTCACCTTGGTCTTCTCTCTTGTGACTCTCTTTGTAGTGGT
TCAACAGCAGACAAGACCTGTTCCGAAGCCTGTGCAAGCCGTAATTCTGGGAGACAACATTACTGGGCATTT
ACCTTTTGAACCCAACAATCATCACCACTTTGGCAGGGAGGCAGAAATGCGAGAGCTTATCCAGACATTTAA
AGGTCACATGGAGAATTCCAGTGCCTGGGTAGTAGAAATCCAGATGTTGAAGTGCAGAGTGGACAATGTCAA
TTCGCAGCTCCAGGTGCTCGGTGATCATCTGGGAAACACCAATGCTGACATCCAGATGGTAAAAGGAGTTCT
AAAGGATGCCACTACATTGAGTTTGCAGACACAGATGTTAAGCAGTTCCCTGGAGGGAACCAATGCTGAGAT
CCAGAGGCTCAAGGAAGACCTTGAAAAGGCAGATGCTTTAACTTTCCAGACGCTGAATTTCTTAAAAAGCAG
TTTAGAAAACACCAGCATTGAGCTCCACGTGCTAAGCAGAGGCTTAGAAAATGCAAACTCTGAAATTCAGAT
GTTGAATGCCACTTTGGAAACGGCAAATGCTTTAAACTCCCAGACCCAGGCCTTTATAAAAAGCAGTTTTGA
CAACACTAGTGCTGAGATCCAGTTCTTAAGAGGTCATTTGGAAAGAGCTGGTGATGAAATTCACGTGTTAAA
AAGGGATTTGAAAATGGTCACAGCCCAGACCCAAAAAGCAAATGGCCGTCTGGACCAGACAGATACTCACAT
TCAGGTATTCAAGTCAGAGATGGAAAATGTGAATACCTTAAATGCCCAGATTCAGGTCTTAAATGGTCATAT
GAAAAATGCCACCAGAGAGATACAGACCCTAAAACAAGGAATGAAGAATGCTTCAGCCTTAACTTCCCAGAC
CCAGATGTTAGACAGCAATCTGCAGAAGGCCAGTGCCCAGATCCAGAGGTTAAGACGGGATCTAGAGAACAC
CAAAGCTCTAACCATGGAAATCCAGCAGGAGCAGAGTCGCCTGAAGACCCTCCATCTGGTCATTACTTCACA
GGAACAGCTACAAAGAACCCAAAGTAAGCAGCTTCTCCAGATGGTCCTGCAAGGCTGGAAGTTCAATGGTGG
AAGCTTATATTATTTTTCTAGTGTCAAGAAGTCTTGGCATGAGGCTGAGCAGTTCTGCGTGTCCCAGGGAGC
CCATCTGGCATCTGTGGCCTCCAAGGAGGACCAGGCATTTCTGGTAGAGTTCACAAGTAAAGTGTACTACTG
GATCGGTCTCACTGACAGGGGCACAGAGGGCTCCTGGCGCTGGACAGATGGGACACCATTCAACGCCGCCCA
GAACAAAGGGTTTTGGGAAAAGAATCAGTCTGACAACTGGCGGCACAAGAATGGGCAGACTGAAGACTGTGT
CCAAATTCAGCAGAAGTGGAATGACATGACCTGTGACACCCCCTATCAGTGGGTGTGCAAGAAGCCCATGGG
CCAGGGTGTGGCCTGAGGGCAGGCCAGAGCTGAGGGGCTGCTCCTGCTTGCCAATACTGACCCTCCTCCTCG
ATGCCTTCGGAGCCTCTGAGCTCTGCTTGTTCTCTGGGACC

In a search of public sequence databases, the NOV42b nucleic acid sequence, located on chromosome 2, has 1054 of 1469 bases (71%) identical to a gb:GENBANK-ID:D88577|acc:D88577.1 mRNA from Mus musculus (mRNA for Kupffer cell receptor, complete cds) (E=1.1e⁻¹¹⁶).

The disclosed NOV42b polypeptide (SEQ ID NO:160) encoded by SEQ ID NO:159 has 549 amino acid residues and is presented in Table 42D using the one-letter amino acid code. Signal P, Psort and/or Hydropathy results predict that NOV42b has a signal peptide and is likely to be localized to the plasma membrane with a certainty of 0.7900. Alternatively, NOV42b may also localize to the microbody (peroxisome) with a certainty of 0.3000, to the Golgi body with a certainty of 0.3000, or to the endoplasmic reticulum (membrane) with a certainty of 0.2000. The most likely cleavage site for NOV42b is between positions 67 and 68: VQQ-QT.

TABLE 42D
Encoded NOV42b protein sequence.

(SEQ ID NO:160)

MKEAEMDGEAVRFCTDNQCVSLHPQEVDSVAMAPAAPKIPRLVQATPAFMAVTLVFSLVTLFVVVQQQTRPV
PKPVQAVILGDNITGHLPFEPNNHHHFGREAEMRELIQTFKGHMENSSAWVVEIQMLKCRVDNVNSQLQVLG
DHLGNTNADIQMVKGVLKDATTLSLQTQMLRSSLEGTNAEIQRLKEDLEKADALTFQTLNFLKSSLENTSIE
LHVLSRGLENANSEIQMLNASLETANALNSQTQAFIKSSFDNTSAEIQFLRGHLERAGDEIHVLKRDLKMVT
AQTQKANGRLDQTDTQIQVFKSEMENVNTLNAQIQVLNGHMKNASREIQTLKQGMKNASALTSQTQMLDSNL
QKASAEIQRLRGDLENTKALTMEIQQEQSRLKTLHVVITSQEQLQRTQSKQLLQMVLQGWKFNGGSLYYFSS
VKKSQHEAEQFCVSQGAHLASVASKEEQAFLVEFTSKVYYWIGLTDRGTEGSWRWTDGTPFNAAQNKGFWEK
NQSDNWRHKNGQTEDCVQIQQKWNDMTCDTPYQWVCKKPMGQGVA

A search of sequence databases reveals that the NOV42b amino acid sequence has 304 of 549 amino acid residues (55%) identical to, and 401 of 549 amino acid residues (73%) similar to, the 548 amino acid residue ptnr:SWISSPROT-ACC:P70194 protein from Mus musculus (Mouse) (Kupffer Cell Receptor) (E=3.7e⁻¹⁵⁸).

NOV42b is predicted to be expressed in at least adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea and uterus.

NOV42c

A disclosed NOV42c nucleic acid of 1874 nucleotides (also referred to as CG56682-03) encoding a Kupffer cell receptor-like protein is shown in Table 42E. An open reading frame was identified beginning with a ATG initiation codon at nucleotides 1-3 and ending with a TAA codon at nucleotides 1702-1704. The start and stop codons are shown in bold in Table 42E, and the 5′ and 3′ untranslated regions, if any, are underlined.

TABLE 42E
NOV42c nucleotide sequence.

(SEQ ID NO:161)

ATGGACGGTGAGGCAGTCCGCTTCTGCACAGATAACCAGTGTGTCTCCCTGCACCCCCAAGAGGTGGACTCT
GTGGCAATGGCTCCTGCAGCCCCCAAGATACCGAGGCTCGTTCAGGCTACCCCGGCATTTATGGCTGTGACC
TTGGTCTTCTCTCTTGTGACTCTCTTTGTAGTGGTTCAACAGCAGACAAGACCTGTTCCGAAGCCTGTGCAA
GCCGTAATTCTGGGAGACAACATTACTGGGCATTTACCTTTTGAACCCAACAATCATCACCACTTTGGCAGG
GAGGCAGAAATGCAAGAGCTTATCCAGACATTTAAAGGCCACATGGAGAATTCCAGTGCCTGGGTAGTAGAA
ATCCAGATGTTGAAGTGCAGAGTGGACAATGTCAATTCGCAGCTCCAGGTGCTCGGTGATCATCTGGGAAAC
ACCAATGCTGACATCCAGATGGTAAAAGGAGTTCTAAAGGATGCCACTACATTGAGTTTGCAGACACAGATG
TTAAGGAGTTCCCTGGAGGGAACCAATGCTGAGATCCAGAGGCTCAAGGAAGACCTTGAAAAGGCAGATGCT
TTAACTTTCCAGACGCTGAATTTCTTAAAAAGCAGTTTAGAAAACACCAGCATTGAGCTCCACGTGCTAAGC
AGAGGCTTAGAAAATGCAAACTCTGAAATTCAGATGTTGAATGCCAGTTTGGAAACGGCAAATACCCAGGCT
CAGTTAGCCAATAGCAGTTTAAAGAACGCTAATGCTGAGATCTATGTTTTGAGAGGCCATCTAGATAGTGTC
AATGACTTGAGGACCCAGAACCAGGTTTTAAGAAATAGTTTGGAAGGAGCCAATGCTGAGATCCAGGGACTA
AAGGAAAATTTGCAGAACACAAATGCTTTAAACTCCCAGACCCAGGCCTTTATAAAAAGCAGTTTTGACAAC
ACTAGTGCTGAGATCCAGTTCTTAAGAGGTCATTTGGAAAGAGCTGGTGATGAAATTCACGTGTTAAAAAGG
GATTTGAAAATGGTCACAGCCCAGACCCAAAAAGCAAATGGCCATCTGGACCAGACAGATACTCAGATTCAG
GTATTCAAGTCAGAGATGGAAAATGTGAATACCTTAAATGCCCAGATTCAGGTCTTAAATGGTCATATGAAA
AATGCCAGCAGAGAGATACAGACCCTAAAACAAGGAATGAAGAATGCTTCAGCCTTAACTTCCCAGACCCAG
ATGTTAGACAGCAATCTGCAGAAGGCCAGTGCCGAGATCCAGAGGTTAAGAGGGGATCTAGAGAACACCAAA
GCTCTAACCATGGAAATCCAGCAGGAGCAGAGTCGCCTGAAGACCCTCCATGTGGTCATTACTTCACAGGAA
CAGCTACAAAGAACCCAAAGTCAGCTTCTCCAGATGGTCCTGCAAGGCTGGAAGTTCAATGGTGGAAGCTTA
TATTATTTTTCTAGTGTCAAGAAGTCTTGGCATGAGGCTGAGCAGTTCTGCGTGTCCCAGGGAGCCCATCTG
GCATCTGTGGCCTCCAAGGAGGAGCAGGCATTTCTGGTAGAGTTCACAAGTAAAGTGTACTACTGGATCGGT
CTCACTGACAGGGGCACAGAGGGCTCCTGGCGCTGGACAGATGGGACACCATTCAACGCCGCCCAGAACAAA
GCCTCCCTAGGAGCCACAGCACCAGGAAGGGATGCTGCCTTCATCTAACAGTATAAAGCCCTGTTGTCTTCG
GGTTTTGGGAAAAGAATCAGTCGACAACTGGCGGCACAAGAATGGGCAGACTGAAGACTGTGTCCAAATTCA
GCAGAAGTGGAATGACATGACCTGTGACACCCCCTATCAGTGGGTGTGCAAGAAGCCCATGGGCCAGGGTGT
GG

In a search of public sequence databases, the NOV42c nucleic acid sequence, located on chromosome 2, has 689 of 993 bases (69%) identical to a gb:GENBANK-ID:D88577|acc:D88577.1 mRNA from Mus musculus (mRNA for Kupffer cell receptor, complete cds) (E=1.6e⁻¹²⁰).

The disclosed NOV42c polypeptide (SEQ ID NO:162) encoded by SEQ ID NO:161 has 567 amino acid residues and is presented in Table 42F using the one-letter amino acid code. Signal P, Psort and/or Hydropathy results predict that NOV42c has a signal peptide and is likely to be localized to the plasma membrane with a certainty of 0.7900. Alternatively, NOV42c may also localize to the microbody (peroxisome) with a certainty of 0.3000, to the Golgi body with a certainty of 0.3000, or to the endoplasmic reticulum (membrane) with a certainty of 0.2000. The most likely cleavage site for NOV42c is between positions 62 and 63: VQQ-QT.

TABLE 42F
Encoded NOV42c protein sequence.

(SEQ ID NO:162)

MDGEAVRFCTDNQCVSLHPQEVDSVAMAPAAPKIPRLVQATPAFMAVTLVFSLVTLFVVVQQQTRPVPKPVQ
AVILGDNITGHLPFEPNNHHHFGREAEMQELIQTFKGHMENSSAWVVEIQMLKCRVDNVNSQLQVLGDHLGN
TNADIQMVKGVLKDATTLSLQTQMLRSSLEGTNAEIQRLKEDLEKADALTFQTLNFLKSSLENTSIELHVLS
RGLENANSEIQMLNASLETANTQAQLANSSLKNANAEIYVLRGHLDSVNDLRTQNQVLRNSLEGANAEIQGL
KENLQNTNALNSQTQAFIKSSFDNTSAEIQFLRGHLERAGDEIHVLKRDLKMVTAQTQKANGHLDQTDTQIQ
VFKSEMENVNTLNAQIQVLNGHMKNASREIQTLKQGMKNASALTSQTQMLDSNLQKASAEIQRLRGDLENTK
ALTMEIQQEQSRLKTLHVVITSQEQLQRTQSQLLQMVLQGWKFNGGSLYYFSSVKKSWHEAEQFCVSQGAHL
ASVASKEEQAFLVEFTSKVYYWIGLTDRGTEGSWRWTDGTPFNAAQNKASLGATAPGRDAAFI

A search of sequence databases reveals that the NOV42c amino acid sequence has 191 of 412 amino acid residues (46%) identical to, and 273 of 412 amino acid residues (66%) similar to, the 548 amino acid residue ptnr:SWISSNEW-ACC:P70194 protein from Mus musculus (Mouse) (Kupffer Cell Receptor) (E=5.3e⁻⁹²).

NOV42c is predicted to be expressed in at least the following tissues: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea and uterus.

NOV42d

A disclosed NOV42d nucleic acid of 1985 nucleotides (also referred to as CG56682-04) encoding a Kupffer cell receptor-like protein is shown in Table 42G. An open reading frame was identified beginning with a GTC initiation codon at nucleotides 2-4 and ending with a TAA codon at nucleotides 1658-1660. The start and stop codons are shown in bold in Table 42G, and the 5′ and 3′ untranslated regions, if any, are underlined. Because the starting codon is not a traditionl initiation codon, NOV42d could be a partial reading frame extending further into the 5′

TABLE 42G
NOV42d nucleotide sequence.

(SEQ ID NO:163)

AGTCCGCTTCTGCACAGATAACCAGTGTGTCTCCCTGCACCCCCAAGAGGTGGACTCTGTGGCAATGGCTCC
TGCAGCCCCCAAGATACCGAGGCTCGTTCAGGCTACCCCGGCATTTATGGCTGTGACCTTGGTCTTCTCTCT
TGTGACTCTCTTTGTAGTGGTTCAACAGCAGACAAGACCTGTTCCGAAGCCTGTGCAAGCCGTAATTCTGGG
AGACAACATTACTGGGCATTTACCTTTTGAACCCAACAATCATCACCACTTTGGCAGGGAGGCAGAAATGCA
AGAGCTTATCCAGACATTTAAAGGCCACATGGAGAATTCCAGTGCCTGGGTAGTAGAAATCCAGATGTTGAA
GTGCAGAGTGGACAATGTCAATTCGCAGCTCCAGGTGCTCGGTGATCATCTGGGAAACACCAATGCTGACAT
CCAGATGGTAAAAGGAGTTCTAAAGGATGCCACTACATTGAGTTTGCAGACACAGATGTTAAGGAGTTCCCT
GGAGGGAACCAATGCTGAGATCCAGAGGCTCAAGGAAGACCTTGAAAAGGCAGATGCTTTAACTTTCCAGAC
GCTGAATTTCTTAAAAAGCAGTTTAGAAAACACCAGCATTGAGCTCCACGTGCTAAGCAGAGGCTTAGAAAA
TGCAAACTCTGAAATTCAGATGTTGAATGCCAGTTTGGAAACGGCAAATACCCAGGCTCAGTTAGCCAATAG
CAGTTTAAAGAACGCTAATGCTGAGATCTATGTTTTGAGAGGCCATCTAGATAGTGTCAATGACTTGAGGAC
CCAGAACCAGGTTTTAAGAAATAGTTTGGAAGGAGCCAATGCTGAGATCCAGGGACTAAAGGAAAATTTGCA
GAACACAAATGCTTTAAACTCCCAGACCCAGGCCTTTATAAAAAGCAGTTTTGGCAACACTAGTGCTGAGAT
CCAGTTCTTAAGAGGTCATTTGGAAAGAGCTGGTGATGAAATTCACGTGTTAAAAAGGGATTTGAAAATGGT
CACAGCCCAGACCCAAAAAGCAAATGGCCGTCTGGACCAGACAGATACTCAGATTCAGGTATTCAAGTCAGA
GATGGAAAATGTGAATACCTTAAATGCCCAGATTCAGGTCTTAAATGGTCATATGAAAAATGCCAGCAGAGA
GATACAGACCCTAAAACAAGGAATGAAGAATGCTTCAGCCTTAACTTCCCAGACCCAGATGTTAGACAGCAA
TCTGCAGAAGGCCAGTGCCGAGATCCAGAGGTTAAGAGGGGATCTAGAGAACACCAAAGCTCTAACCATGGA
AATCCAGCAGGAGCAGAGTCGCCTGAAGACCCTCCATGTGGTCATTACTTCACAGGAACAGCTACAAAGAAC
CCAAAGTCAGCTTCTCCAGATGGTCCTGCAAGGCTGGAAGTTCAATGGTGGAAGCTTATATTATTTTTCTAG
TGTCAAGAAGTCTTGGCATGAGGCTGAGCAGTTCTGCGTGTCCCAGGGAGCCCATCTGGCATCTGTGGCCTC
CAAGGAGGAGCAGGCATTTCTGGTAGAGTTCACAAGTAAAGTGTACTACTGGATCGGTCTCACTGACAGGGG
CACAGAGGGCTCCTGGCGCTGGACAGATGGGACACCATTCAACGCCGCCCAGAACAAAGCGGCCACTAGGGG
ATGAAGGACCCATCTCAAGTCAGCTCCCTAGACTCATCCCATGTCAGCTCCCTAGGAGCCACAGCACCAGGA
AGGGATGCTGCCTTCATCTAACAGTATAAAGCCCTGTTGTCTTCGGGTTTTGGGAAAAGAATCAGTCTGACA
ACTGGCGGCACAAGAATGGGCAGACTGAAGACTGTGTCCAAATTCAGCAGAAGTGGAATGACATGACCTGTG
ACACCCCCTATCAGTGGGTGTGCAAGAAGCCCATGGGCCAGGGTGTGGCCTGAGGGCAGGCCAGAGCTGAGG
GGCTGCTCCTGCTTGCCAATACTGACCCTCCTCCTCGATGC

In a search of public sequence databases, the NOV42d nucleic acid sequence, located on chromosome 2, has 705 of 1023 bases (68%) identical to a gb:GENBANK-ID:D88577|acc:D88577.1 mRNA from Mus musculus (mRNA for Kupffer cell receptor, complete cds) (E=3.7e⁻¹²⁴).

The disclosed NOV42d polypeptide (SEQ ID NO:164) encoded by SEQ ID NO:163 has 552 amino acid residues and is presented in Table 42H using the one-letter amino acid code. Signal P, Psort and/or Hydropathy results predict that NOV42d has a signal peptide and is likely to be localized to the plasma membrane with a certainty of 0.7900. Alternatively, NOV42d may also localize to the microbody (peroxisome) with a certainty of 0.3000, to the Golgi body with a certainty of 0.3000, or to the endoplasmic reticulum (membrane) with a certainty of 0.2000. The most likely cleavage site for NOV42d is between positions 57 and 58: VQQ-QT.

TABLE 42H
Encoded NOV42d protein sequence.

(SEQ ID NO:164)

VRFCTDNQCVSLHPQEVDSVAMAPAAPKIPRLVQATPAFMAVTLVFSLVTLFVVVQQQTRPVPKPVQAVILG
DNITGHLPFEPNNHHHFGREAEMQELIQTFKGHMENSSAWVVEIQMLKCRVDNVNSQLQVLGDHLGNTNADI
QMVKGVLKDATTLSLQTQMLRSSLEGTNAEIQRLKEDLEKADALTFQTLNFLKSSLENTSIELHVLSRGLEN
ANSEIQMLNASLETANTQAQLANSSLKNANAEIYVLRGHLDSVNDLRTQNQVLRNSLEGANAEIQGLKENLQ
NTNALNSQTQAFIKSSFGNTSAEIQFLRGHLERAGDEIHVLKRDLKMVTAQTQKANGRLDQTDTQIQVFKSE
MENVNTLNAQIQVLNGHMKNASREIQTLKQGMKNASALTSQTQMLDSNLQKASAEIQRLRGDLENTKALTME
IQQEQSRLKTLHVVITSQEQLQRTQSQLLQMVLQGWKFNGGSLYYFSSVKKSWHEAEQFCVSQGAHLASVAS
KEEQAFLVEFTSKVYYWIGLTDRGTEGSWRWTDGTPFNAAQNKAATRG

A search of sequence databases reveals that the NOV42d amino acid sequence has 187 of 404 amino acid residues (46%) identical to, and 269 of 404 amino acid residues (66%) similar to, the 548 amino acid residue ptnr:SWISSNEW-ACC:P70194 protein from Mus musculus (Mouse) (Kupffer Cell Receptor) (E=1.2e⁻⁸⁹).

NOV42d is predicted to be expressed in at least adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea and uterus.

NOV42a also has homology to the amino acid sequences shown in the BLASTP data listed in Table 42I.

TABLE 42I
BLAST results for NOV42a

| Gene Index/ | | Length | Identity | Positives | |
|---|---|---|---|---|---|
| Identifier | Protein/Organism | (aa) | (%) | (%) | Expect |
| gi\|7949066\|ref\|NP_058031.1\| | C-type (calcium | 548 | 300/548 | 395/548 | e−153 |
| (NM_016751) | dependent, | | (54%) | (71%) | |
| | carbohydrate | | | | |
| | recognition | | | | |
| | domain) lectin, | | | | |
| | superfamily | | | | |
| | member 13; | | | | |
| | kupffer cell | | | | |
| | receptor; Kupffer | | | | |
| | cell c-type | | | | |
| | lectin receptor | | | | |
| | [Mus musculus] | | | | |
| gi\|16758588\|ref\|NP_{—} | Kupffer cell | 550 | 293/547 | 382/547 | e−147 |
| 446205.1\| | receptor [Rattus | | (53%) | (69%) | |
| (NM_053753) | norvegicus] | | | | |
| gi\|7657291\|ref\|NP_056532.1\| | Langerhans cell | 328 | 92/261 | 143/261 | 2e−41 |
| (NM_015717) | specific c-type | | (35%) | (54%) | |
| | lectin; langerin | | | | |
| | [Homo sapiens] | | | | |
| gi\|17426713\|emb\|CAC85632.1\| | langerin [Mus | 326 | 91/264 | 140/264 | 1e−40 |
| (AJ313164) | musculus] | | (34%) | (52%) | |
| gi\|17059581\|emb\|CAC82936.1\| | C type lectin | 331 | 91/264 | 140/264 | 2e−40 |
| (AJ302711) | [Mus musculus] | | (34%) | (52%) | |

The homology between these and other sequences is shown graphically in the ClustalW analysis shown in Table 42J. In the ClustalW alignment of the NOV42 protein, as well as all other ClustalW analyses herein, the black outlined amino acid residues indicate regions of conserved sequence (i.e., regions that may be required to preserve structural or functional properties), whereas non-highlighted amino acid residues are less conserved and can potentially be altered to a much broader extent without altering protein structure or function.

[image]

[image]

[image]

Tables 42K-N list the domain descriptions from DOMAIN analysis results against NOV42. This indicates that the NOV42 sequence has properties similar to those of other proteins known to contain this domain.

TABLE 42K
Domain Analysis of NOV42a

gnl|Smart|smart00034, CLECT, C-type lectin (CTL) or carbohydrate
recognition domain (CRD); Many of these domains function as calcium-dependent
carbohydrate binding modules. (SEQ ID NO:836)
CD-Length = 124 residues, 98.4% aligned
Score = 124 bits (311), Except = 1e−29

| | | | |
|---|---|---|---|
| NOV42: | 415 | QGWK-FNGGSLYYFSSVKKSWHEAEQFCVSQGAHLASVASKEEQAFLVEFTSKV---YYW | 470 |
| | | \|\| + \|\| \| \|\|+ \|\|+\| +\|+ \|\| \| \|\|\|\|\|\|+ \|+\|\| \|\|+ \|\|\| | 470 |
| Sbjct: | 3 | SGWVSYPGGKCYKFSTEKKTWADAQAFCQSLGAHLASIHSEEENDFLLSLLKNSNSDYYW | 62 |
| NOV42: | 471 | IGLTDRGTEGSWRWTDGTPFNAAQNKGFWEKNQSDNWRHKNGQTEDCVQIQQ----KWND | 526 |
| | | \|\|\|+ + \|\|\|+\|+\|\|+ \| \| + + +\|\| + \|\|\|\| | 526 |
| Sbjct: | 63 | IGLSRPDSNGSWQWSDGSGPVDYSN---WAPGEPGG-------SGNCVVLSTSGGGKWND | 112 |
| NOV42: | 527 | MTCDTPYQWVCK 538 | |
| | | ++\| + ++\|+ | 538 |
| Sbjct: | 113 | VSCTSKLPFICE 124 | |

TABLE 42L
Domain Analysis of NOV42a

gnl|Pfam|pfam00059, lectin_c, Lectin C-type domain. This family
includes both long and short form C-type (SEQ ID NO:837)
CD-Length = 107 residues, 99.1% aligned
Score = 115 bits (288), Expect = 6e −27

| | | | |
|---|---|---|---|
| NOV42: | 431 | KKSWhEAEQFCVSQGAHLASVASKEEQAFLVEFT--SKVYYWIGLTDRGTEGSWRWTDGT | 488 |
| | | \|+\| \|\|+ \| \| \| \|+ \| \|\|\| \|\| \| \| \| \|\|\|\|\|\| \|\|\|+\| \|\|\|\|+ | |
| Sbjct: | 2 | SKTWAEAQAACQKLGGGLVSIQSAEEQDFLTSLTKASNSYAWIGLTDINTEGTWVWTDGS | 61 |
| NOV42: | 489 | PFNAAQNKGFWEKNQSDNWRHKNGQTEDCVQIQ---QKWNDMTCDTPYQWVCKK 539 | |
| | | \| \| \| + +\| +\| \|\|\|\|+\| \|\|\|\| \| + +\|\|+ | |
| Sbjct: | 62 | PVNYT----NWAPGEPNNRGNK----EDCVEIYTDGNKWNDEPCGSKLPYVCEF 107 | |

TABLE 42M
Domain Analysis of NOV42a

gnl|Pfam|pfam01576, Myosin_tail, Myosin tail. The myosin molecule is a
multi-subunit complex made up of two heavy chains and four light
chains it is a fundamental contractile protein found in all eukaryote
cell types. This family consists of the coiled-coil myosin heavy chain
tail region. The coiled-coil is composed of the tail from two
molecules of myosin. These can then assemble into the macromolecular
thick filament. The coiled-coil region provides the structural
backbone the thick filament. (SEQ ID NO:838)
CD-Length = 860 residues, 29.4% aligned
Score = 43.1 bits (100), Expect = 4e −05

| | | | |
|---|---|---|---|
| NOV42: | 121 | VEIQMLKCRVDNVNSQLQVLGDHLGNTNADIQMVKGVLKDATTLSLQTQMLRSSLEGTNA | 180 |
| | | ++ \|+ ++\| + \|\| \| ++ + \|++\| \|+\| \|\| | |
| Sbjct: | 187 | SQLSELQVKLDELQRQLNDLTSQKSRLQSENSDLTRQLEEAEAQVSNLSKLKSQLESQLE | 246 |
| NOV42: | 181 | EIQRLKEDLEKADALTFQTLNFLKSSLENTSIELHVLSRGLENANSEIQMLNASLETANA | 240 |
| | | \| +\| \|+ + \| \|++ \| +\| \| \|\| + \| \| \|\|\| | |
| Sbjct: | 247 | EAKRSLEEESRERAN-------LQAQLRQLEHDLDSLREQLEEESEAKAELERQLSKANA | 299 |
| NOV42: | 241 | LNSQTQAFIKSSFDNTSAEIQFLRGHLERAGDEIHVLKRDLKMVTAQTQKANGRLDQTDT | 300 |
| | | \| ++ +\| + \|++ \|+ \| + \|+ +\| \|\| | |
| Sbjct: | 300 | EIQQWRSKFESEGALRAEELEELKKKLNQKISELEEAAEAANAKCDSLEKTKSRLQS--- | 356 |
| NOV42: | 301 | QIQVFKSEMENVNTLNAQIQVLNGHMKNASREIQTLKQGMKNASALTSQTQMLDSNLQKA | 360 |
| | | +++ + \|+\| \| ++ \| \|\| + + \| + \| ++ + | |
| Sbjct: | 357 | ELEDLQIELERANAAASE---LEKKQKNFDKILAEWK---RKVDELQAELDTAQREARNL | 410 |
| NOV42: | 361 | SAEIQRLRGDLENTKALTMEIQQEQSRLK 389 | |
| | | \| \|+ \|\|+ +\|\| \| +++\| \|+ | |
| Sbjct: | 411 | STELFRLKNELEELKDQVEALRRENKNLQ 439 | |

TABLE 42N
Domain Analysis of NOV42c

gnl|Pfam|pfam01576, Myosin_tail, Myosin tail. The myosin molecule is a
multi-subunit complex made up of two heavy chains and four light
chains it is a fundamental contractile protein found in all eukaryote
cell types. This family consists of the coiled-coil myosin heavy chain
tail region. The coiled-coil is composed of the tail from two
molecules of myosin. These can then assemble into the macromolecular
thick filament. The coiled-coil region provides the structural
backbone the thick filament. (SEQ ID NO:838)
CD-Length = 860 residues, 30.2% aligned
Score = 39.3 bits (90), Expect = 6e −04

| | | | |
|---|---|---|---|
| NOV42: | 135 | SQLQVLGDHLGNTNADIQMVKGVLKDATTLSLQTQMLRSSLEGTNAEIQRLKEDLEKADA | 194 |
| | | ++\|++ \|\| \|\|+ \| \|+ \| + \|++ +\| \| \|\| +\|\| + \| | |
| Sbjct: | 556 | NELEIALDHANKANAEAQ------KNVKKYQQQVKELQTQVE----EEQRAREDAREQLA | 605 |
| NOV42: | 195 | LTFQTLNFLKSSLENTSIELHVLSRGLENANSEIQMLNASLETANALNSQTQAFIKSSFD | 254 |
| | | + + \|++ \|\| \| \| + \| +\| \| + \| \| \| +\| + \| | |
| Sbjct: | 606 | VAERRATALEAELEELRSALEQAERARKQAETE---LAEASERVNELTAQNSSLIAQK-R | 661 |
| NOV42: | 255 | NTSAEIQFLRGHLERAGDEIHVLKRDLKMVTAQTQKANGRLDQTDTQIQVFKSEMENVNT | 314 |
| | | \|+ \|+ \|+ \| +\|+ + + +\|\| + +++ + +++ | |
| Sbjct: | 662 | KLEGELAALQSDLDEAVNELKAAEE-------RAKKAQADAARLAEELRQEQEHSQHLER | 714 |
| NOV42: | 315 | LNAQIQVLNGHMKNASREIQT--LKQGMKNASALTSQTQMLDSNL---QKASAEIQR-LR | 368 |
| | | \| \|++ ++ \| + \|\| \| \| \| ++ + \|++ \| \|+ \|\| \|+ \|\| | |
| Sbjct: | 715 | LRKQLESQVKELQVRLDEAEAAALKGGKKMIQKLEARVRELEAELDGEQRRHAETQKNLR | 774 |
| NOV42: | 369 | GDLENTKALTMEIQQEQSRLKTLHVVITSQEQLQRTQSKQL 409 | |
| | | \| \| ++++++ \|+ \| ++ + +\| +\|\| | |
| Sbjct: | 775 | KMERRVKELQFQVEEDKKNLERLQDLVDKLQAKIKTYKRQL 815 | |

Kupffer cells are found in the linings of the liver sinusoids, and are phagocytic. A receptor uniquely found on the surface of rat Kupffer cells binds oligosaccharides terminating in galactose, N-acetylgalactosamine, and fucose. A number of different families of proteins share a conserved domain which was first characterized in some animal lectins. Animal lectins display a wide variety of architectures. They are classified according to the carbohydrate-recognition domain (CRD) of which there are two main types, S-type and C-type. C-type lectins (CTL) display a wide range of specificities and function as a calcium-dependent carbohydrate-recognition domain. They are found predominantly but not exclusively in vertebrates. CTLs can be classified into a number of subgroups based on their function and structure: 1) Collectins, represented by the soluble mannose-binding proteins of mammalian serum and liver; 2) Selectins, membrane-bound proteins involved in inflammation; and 3) Endocytic lectins, membrane-bound receptors that mediate endocytosis of glycoproteins. Endocytic lectins are type-II membrane proteins where the CTL domain is located at the C-terminal extremity of the proteins, and include the Kupffer Cell Receptor.

The disclosed NOV42 nucleic acid of the invention encoding a Kupffer Cell Receptor-like protein includes the nucleic acid whose sequence is provided in Table 42A, 42C, 42E, 42G, or a fragment thereof. The invention also includes a mutant or variant nucleic acid any of whose bases may be changed from the corresponding base shown in Table 42A, 42C, 42E, or 42G while still encoding a protein that maintains its Kupffer Cell Receptor-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 32 percent of the bases may be so changed.

The disclosed NOV42 protein of the invention includes the Kupffer Cell Receptor-like protein whose sequence is provided in Table 42B,42D, 42F, or 42H. The invention also includes a mutant or variant protein any of whose residues may be changed from the corresponding residue shown in Table 42B, 42D, 42F, or 42H while still encoding a protein that maintains its Kupffer Cell Receptor-like activities and physiological functions, or a functional fragment thereof. In the mutant or variant protein, up to about 66 percent of the residues may be so changed.

The invention further encompasses antibodies and antibody fragments, such as F_{ab} or (F_{ab})₂, that bind immunospecifically to any of the proteins of the invention.

The above disclosed information suggests that this Kupffer Cell Receptor-like protein (NOV42) is a member of a “Kupffer Cell Receptor family”. Therefore, the NOV42 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV42 nucleic acids and proteins of the invention are useful in potential therapeutic applications implicated in Von Hippel-Lindau (VHL) syndrome, cirrhosis, transplantation, arthritis, tendinitis, and/or other diseases and pathologies.

NOV42 nucleic acids and polypeptides are further useful in the generation of antibodies that bind immuno-specifically to the novel NOV42 substances for use in therapeutic or diagnostic methods. These antibodies may be generated according to methods known in the art, using prediction from hydrophobicity charts, as described in the “Anti-NOVX Antibodies” section below. The disclosed NOV42 protein has multiple hydrophilic regions, each of which can be used as an immunogen. These novel proteins can be used in assay systems for functional analysis of various human disorders, which will help in understanding of pathology of the disease and development of new drug targets for various disorders.

NOV43

A disclosed NOV43 nucleic acid of 1108 nucleotides (also referred to as CG56690-01) encoding a P2Y Purinoceptor-like protein is shown in Table 43A. An open reading frame was identified beginning with a ATG initiation codon at nucleotides 12-14 and ending with a TAA codon at nucleotides 1095-1097. The start and stop codons are shown in bold in Table 43A, and the 5′ and 3′ untranslated regions, if any, are underlined.

TABLE 43A
NOV43 nucleotide sequence.

(SEQ ID NO:165)

GTCATGATGTTATGCTGTCCATTTTGCTTCCTTCCAGGGGAAGCAGAAGCGGGAGCCGTCGTGGAGCTCTGC
TCCTGGAGGGAGCCTCCCGGGACATGGAGAAGGTGGACATGAATACATCACAGGAACAAGGTCTCTGCCAGT
TCTCAGAGAAGTACAAGCAAGTCTACCTCTCCCTGGCCTACAGTATCATCTTTATCCTAGGGCTGCCACTAA
ATGGCACTGTCTTGTGGCACTCCTGGGGCCAAACCAAGCGCTGGAGCTGTGCCACCACCTATCTGGTGAACC
TGATGGTGGCCGACCTGCTTTATGTGCTATTGCCCTTCCTCATCATCACCTACTCACTAGATGACAGGTGGC
CCTTCGGGGAGCTGCTCTGCAAGCTGGTGCACTTCCTGTTCTATATCAACCTTTACGGCAGCATCCTGCTGC
TGACCTGCATCTCTGTGCACCAGTTCCTAGGTGTGTGCCACCCACTGTGTTCGCTGCCCTACCGGACCCGCA
GGCATGCCTGGCTGGGCACCAGCACCACCTGGGCCCTGGTGGTCCTCCAGCTGCTGCCCACACTGGCCTTCT
CCCACACGGACTACATCAATGGCCAGATGATCTGGTATGACATGACCAGCCAAGAGAATTTTGATCGGCTTT
TTGCCTACGGCATAGTTCTGACATTGTCTGGCTTTCTTTCCCTCCTTGGTCATTTTGGTGTGTATTCACTGA
TGGTCAGGAGCCTGATCAAGCCAGAGGAGAACCTCATGAGGACAGGCAACACAGCCCGAGCCAGGTCCATCC
GGACCATCCTACTGGTGTGTGGCCTCTTCACCCTCTGTTTTGTGCCCTTCCATATCACTCGCTCCTTCTACC
TCACCATCTGCTTTCTGCTTTCTCAGGACTGCCAGCTCTTGATGGCAGCCCAGTGTGGCCTACAAGATATGG
AGGCCTCTGGTGAGTGTGAGCAGCTGCCTCAACCCAGTCCTGTACTTTCTTTCAAGGGGGGCAAAAATAGAG
TCAGGCTCCTCCAGAAACTGAGGCAGAACAAGTTGGGTGAGCATCCAGCTGGGAGGAAGAGATGCCCAGGGT
TGAACAGATCTGGGTAATGCCAAGGTGA

In a search of public sequence databases, the NOV43 nucleic acid sequence, located on chromosome 2, has 585 of 924 bases (63%) identical to a gb:GENBANK-ID:GDP2Y3|acc:X98283.1 mRNA from Gallus gallus (G.domesticus mRNA for G protein-coupled P2 receptor) (E=3.6e⁻⁴⁵).

The disclosed NOV43 polypeptide (SEQ ID NO:166) encoded by SEQ ID NO:165 has 361 amino acid residues and is presented in Table 43B using the one-letter amino acid code. Signal P, Psort and/or Hydropathy results predict that NOV43 has a signal peptide and is likely to be localized to the plasma membrane with a certainty of 0.6000. Alternatively, NOV43 may also localize to the mitochondrial inner membrane with a certainty of 0.5862, to the mitochondrial intermembrane space with a certainty of 0.4114, or to the Golgi body with a certainty of 0.4000. The most likely cleavage site for NOV43 is between positions 13 and 14: SRS-GS.

TABLE 43B
Encoded NOV43 protein sequence.

(SEQ ID NO:166)

MLSILLPSRGSRSGSRRGALLLEGASRDMEKVDMNTSQEQGLCQFSEKYKQVYLSLAYSIIFILGLPLNGTV
LWHSWGQTKRWSCATTYLVNLMVADLLYVLLPFLIITYSLDDRWPFGELLCKLVHFLFYINLYGSILLLTCI
SVHQFLGVSHPLCSLPYRTRRHAWLGTSTTWALVVLQLLPTLAFSHTDYINGQMIWYDMTSQENFDRLFAYG
IVLTLSGFLSLLGHFGVYSLMVRSLIKPEENLMRTGNTARARSIRTILLVCGLFTLCFVPFHITRSFYLTIC
FLLSQDCQLLMAAQCGLQDMEASGECEQLPQPSPVLSFKGGKNRVRLLQKLRQNKLGEHPAGRKRCPGLNRSG

A search of sequence databases reveals that the NOV43 amino acid sequence has 105 of 261 amino acid residues (40%) identical to, and 153 of 261 amino acid residues (58%) similar to, the 328 amino acid residue ptnr:SWISSNEW-ACC:Q98907 protein from Gallus gallus (Chicken) (P2Y Purinoceptor 3 (P2Y3) (Nucleoside Diphosphate Receptor)) (E=0.0).

NOV43 is predicted to be expressed in brain because of the expression pattern of (GENBANK-ID: gb:GENBANK-ID:GDP2Y3|acc:X98283.1) a closely related G.domesticus mRNA for G protein-coupled P2 receptor homolog in species Gallus gallus.

NOV43 also has homology to the amino acid sequences shown in the BLASTP data listed in Table 43C.

TABLE 43C
BLAST results for NOV43

| Gene Index/ | | Length | Identity | Positives | |
|---|---|---|---|---|---|
| Identifier | Protein/Organism | (aa) | (%) | (%) | Expect |
| gi\|2829680\|sp\|P79928\| | P2Y PURINOCEPTOR | 537 | 111/259 | 154/259 | 5e−49 |
| P2Y8_XENLA | 8 (P2Y8) | | (42%) | (58%) | |
| gi\|2707256\|gb\|AAC60339.1\| | G protein coupled | 374 | 107/277 | 158/277 | 4e−46 |
| (AF031897) | P2Y nucleotide | | (38%) | (56%) | |
| | receptor | | | | |
| | [Meleagris | | | | |
| | gallopavo] | | | | |
| gi\|2495017\|sp\|Q98907\| | P2Y PURINOCEPTOR | 328 | 105/261 | 153/261 | 6e−45 |
| P2Y3_CHICK | 3 (P2Y3) | | (40%) | (58%) | |
| | (NUCLEOSIDE | | | | |
| | DIPHOSPHATE | | | | |
| | RECEPTOR) | | | | |
| gi\|10720180\|sp\|093361\| | P2Y PURINOCEPTOR | 328 | 105/269 | 155/269 | 2e−44 |
| P2Y3_MELGA | 3 (P2Y3) | | (39%) | (57%) | |
| | (NUCLEOSIDE | | | | |
| | DIPHOSPHATE | | | | |
| | RECEPTOR) | | | | |
| gi\|13928944\|ref\|NP_{—} | purinergic | 361 | 118/310 | 169/310 | 6e−44 |
| 113868.1\| | receptor P2Y, G- | | (38%) | (54%) | |
| (NM_031680) | protein coupled, | | | | |
| | 4; pyrimidinergic | | | | |
| | receptor P2Y, G- | | | | |
| | protein coupled, | | | | |
| | 4 [Rattus | | | | |
| | norvegicus] | | | | |

The homology between these and other sequences is shown graphically in the ClustalW analysis shown in Table 43D. In the ClustalW alignment of the NOV43 protein, as well as all other ClustalW analyses herein, the black outlined amino acid residues indicate regions of conserved sequence (i.e., regions that may be required to preserve structural or functional properties), whereas non-highlighted amino acid residues are less conserved and can potentially be altered to a much broader extent without altering protein structure or function.

[image]

[image]

Tables 43E lists the domain descriptions from DOMAIN analysis results against NOV43. This indicates that the NOV43 sequence has properties similar to those of other proteins known to contain this domain.

TALBE 43E
Domain Analysis of NOV43

gnl|Pfam|pfam00001, 7tm_1, 7 transmembrane receptor (rhodopsin
family). (SEQ ID NO:810)
CD-Length = 254 residues, 85.0% aligned
Score = 111 bits (277), Expect = 83 −26

| | | | |
|---|---|---|---|
| NOV43: | 69 | NGTVLWHSWGQTKRWSCATTYLVNLMVADLLYVL-LPFLIITYSLDDRWPFGELLCKLVH | 127 |
| | | \| \|+ \| + +\|+\|\| \|\|\|\|\|++\| \|\| + \| + \| \|\|+ \|\|\|\|\| | |
| Sbjct: | 2 | NLLVILVILRTKKLRTPTNIFLLNLAVADLLFLLTLPPWALYYLVGGDWVFGDALCKLVG | 61 |
| NOV43: | 128 | FLFYINLYGSILLLTCISVHQFLGVCHPLCSLPYRTRRHAWLGTSTTWALVVLQ-LLPTL | 186 |
| | | \|\| +\| \| \|\|\|\|\|\| \|\|+ ++\| + \|\|\| \|\| \| \| + \| \| +51 \| \| \| | |
| Sbjct: | 62 | ALFVVNGYASILLLTAISIDRYLAIVHPLRYRRIRTPRRAKVLILLVWVLALLLSLPPLL | 121 |
| NOV43: | 187 | AFSHTDYINGQMIWYDMTSQENFDRLFAYGIVLTLSGFLSLLGHFGVYSLMVRSLIKPE- | 245 |
| | | \| + \| + + + \| \|\| \|+ ++\|+\| \| | |
| Sbjct: | 122 | FSWLRTVEEGNTTVCLIDFPEESVKRSYVLLSTLVGFVLPLLVILVCYTRILRTLRKRAR | 181 |
| NOV43: | 246 | -ENLMRTGNTARARSIRTILLVCGLFTLCFVPFHIT 280 | |
| | | + ++ +++ ++ + +\|+\| +\| \|\|++\|+\|\| | |
| Sbjct: | 182 | SQRSLKRRSSSERKAAKMLLVVVVVFVLCWLPYHIV 217 | |

The P2Y Purinoreceptor belongs to the family of G-Protein Coupled Receptors. G-protein-coupled receptors (GPCRs) constitute a vast protein family that encompasses a wide range of functions (including various autocrine, paracrine and endocrine processes). They show considerable diversity at the sequence level, on the basis of which they can be separated into distinct groups. We use the term clan to describe the GPCRs, as they embrace a group of families for which there are indications of evolutionary relationship, but between which there is no statistically significant similarity in sequence [1]. The currently known clan members include the rhodopsin-like GPCRs, the secretin-like GPCRs, the cAMP receptors, the fungal mating pheromone receptors, and the metabotropic glutamate receptor family. The rhodopsin-like GPCRs themselves represent a widespread protein family that includes hormone, neurotransmitter and light receptors, all of which transduce extracellular signals through interaction with guanine nucleotide-binding (G) proteins. Although their activating ligands vary widely in structure and character, the amino acid sequences of the receptors are very similar and are believed to adopt a common structural framework comprising 7 transmembrane (TM) helices. See CMKRL2 (601805). Using degenerate PCR to find cDNAs encoding new G protein coupled-receptors in human B cells, Owman et al. (1996) identified a CMKRL1 cDNA which encodes a 352-amino acid polypeptide with a calculated mass of 43 kD. The nearest homologs of this novel sequence are the chemoattractant leukocyte receptors, such as the C5a anaphylatoxin receptor and the FMLP receptor. Northern blotting revealed transcripts of 5 kb and 7.5 kb in several tissues of the immune system including spleen, thymus, and lymph node. Owman et al. (1996) considered the high level of expression in lymphoid tissues suggestive of the role of CMKRL1 in the regulation of the inflammatory system. The authors mapped the CMKRL1 gene to 14q11.2-q12 by fluorescence in situ hybridization. Akbar et al. (1996) used a chicken P2Y3 cDNA to screen a human erythroleukemia (HEL) cell cDNA library and cloned a purinoceptor cDNA, which they termed P2Y7. Sequencing revealed an open reading frame coding for a polypeptide of 352 amino acids having 7 putative transmembrane domains. The P2Y7 receptor has 23 to 30% identity to other P2Y receptors, but forms a unique branch within the P2Y family. Northern blot analysis showed that the P2Y7 gene produced a 1.6-kb transcript which is expressed at highest levels in human heart, human skeletal muscle, rat heart, and rat cardiomyocytes and at lower levels in human brain and human liver. Akbar et al. (1996) noted that its expression in HEL cells is below the threshold of detection by Northern blot. Binding and displacement assays in COS-7 cells showed that P2Y7 has a high affinity for ATP and much less for UTP and ADP. The rank order of affinities in the binding series was distinct from any known for the P2Y1-P2Y6 receptors. Like other P2Y receptors, P2YR is coupled to phospholipase C and not to adenylate cyclase. Akbar et al. (1996) speculated that P2Y7 may be the cardiac P2Y receptor involved in the regulation of cardiac muscle contraction through modulation of L-type calcium currents. Akbar et al. (1996) used PCR on a panel of mouse-rodent somatic cell hybrids to localize the P2RY7 gene to human chromosome 14. Somers et al. (1997) did sequence tagged site (STS) mapping of the P2RY7 gene using the National Center for Biotechnology Information (NCBI) database. In this way, they positioned the P2RY7 gene between D14S283 and D14S264. Leukotriene B4 (LTB4) is a potent chemoattractant that is primarily involved in inflammation, immune responses, and host defense against infection (Samuelsson et al., 1987; Chen et al., 1994). LTB4 activates inflammatory cells by binding to its cell surface receptor, BLTR. LTB4 can also bind and activate the intranuclear transcription factor PPAR-alpha, resulting in the activation of genes that terminate inflammatory processes (Devchand et al., 1996). Yokomizo et al. (1997) cloned the cDNA encoding a cell surface LTB4 receptor that is highly expressed in human leukocytes. Two cDNA clones isolated from retinoic acid-differentiated HL-60 cells contained identical open reading frames encoding a protein of 352 amino acids and predicted to contain 7 membrane-spanning domains, but different 5-prime untranslated regions. In Chinese hamster ovary (CHO) cells stably expressing this receptor, LTB4 induced increases in intracellular calcium, accumulation of D-myo-inositol-1,4,5-triphosphate, and inhibition of adenylyl cyclase. Furthermore, CHO cells expressing exogenous BLTR showed marked chemotactic responses toward low concentrations of LTB4 in a pertussis-toxin-sensitive manner. Yokomizo et al. (1997) found that the putative purinoceptor P2Y7 has a primary structure identical to that of one of the BLTR clones, HL-5. To determine whether BLTR also functions as a purinoceptor, they established stable transformants of BLTR in glioma cells that possess negligible amounts of intrinsic purinoceptors. In these cells, up to 300 microM caused no change in intracellular calcium levels, but significant increases in the calcium concentrations were induced by exposure to 10 nanoM LTB4. These results were interpreted to indicate that this receptor is not a purinoceptor, but a BLTR.

The disclosed NOV43 nucleic acid of the invention encoding a P2Y Purinoceptor-like protein includes the nucleic acid whose sequence is provided in Table 43A or a fragment thereof. The invention also includes a mutant or variant nucleic acid any of whose bases may be changed from the corresponding base shown in Table 43A while still encoding a protein that maintains its UDP[Glycosyltransferase-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 37 percent of the bases may be so changed.

The disclosed NOV43 protein of the invention includes the P2Y Purinoceptor-like protein whose sequence is provided in Table 43B. The invention also includes a mutant or variant protein any of whose residues may be changed from the corresponding residue shown in Table 43B while still encoding a protein that maintains its P2Y Purinoceptor-like activities and physiological functions, or a functional fragment thereof. In the mutant or variant protein, up to about 62 percent of the residues may be so changed.

The invention further encompasses antibodies and antibody fragments, such as F_{ab} or (F_{ab})₂, that bind immunospecifically to any of the proteins of the invention.

The above disclosed information suggests that this P2Y Purinoceptor-like protein (NOV43) is a member of a “P2Y Purinoceptor family”. Therefore, the NOV43 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV43 nucleic acids and proteins of the invention are useful in potential therapeutic applications implicated in Von Hippel-Lindau (VHL) syndrome, Alzheimer's disease, Stroke, Tuberous sclerosis, hypercalceimia, Parkinson's disease, Huntington's disease, Cerebral palsy, Epilepsy, Lesch-Nyhan syndrome, Multiple sclerosis, Ataxia-telangiectasia, Leukodystrophies, Behavioral disorders, Addiction, Anxiety, Pain, Neuroprotection, and/or other diseases and pathologies.

NOV43 nucleic acids and polypeptides are further useful in the generation of antibodies that bind immuno-specifically to the novel NOV43 substances for use in therapeutic or diagnostic methods. These antibodies may be generated according to methods known in the art, using prediction from hydrophobicity charts, as described in the “Anti-NOVX Antibodies” section below. The disclosed NOV43 protein has multiple hydrophilic regions, each of which can be used as an immunogen. These novel proteins can be used in assay systems for functional analysis of various human disorders, which will help in understanding of pathology of the disease and development of new drug targets for various disorders.

NOV44

A disclosed NOV44 nucleic acid of 934 nucleotides (also referred to as CG56692-01) encoding a G Protein Coupled Receptor-like protein is shown in Table 44A. An open reading frame was identified beginning with a ATG initiation codon at nucleotides 15-17 and ending with a TAA codon at nucleotides 921-923. The start and stop codons are shown in bold in Table 44A, and the 5′ and 3′ untranslated regions, if any, are underlined.

TABLE 44A
NOV44 nucleotide sequence.

(SEQ ID NO:167)

TGACCTTGGAATCTATGGACATACCACAAAATATCACAGAATTTTTCATGCTGGGGCTCTCACAGAACTCAG
AGGTACAGAGAGTTCTCTTTGTGGTCTTTTTGCTGATCTATGTGGTCACGGTTTGTGGCAACATGCTCATTG
TGGTCACTATCACCTCCAGCCCCACGCTGGCTTCCCCTGTGTATTTTTTCCTGGCCAACCTATCCTTTATTG
ACACCTTTTATTCTTCTTCTATGGCTCCTAAACTCATTGCTGACTCATTGTATGAGGGGAGAACCATCTCTT
ATGAGTGCTGCATGGCTCAGCTCTTTGGAGCTCATTTTTTGGGAGGTGTTGAGATCATTCTGCTCACAGTGA
TGGCTTATGACCGCTATGTGGCCATCTGTAAGCCCCTGCACAATACTACCATCATGACCAGGCATCTCTGTG
CCATGCTTGTAGGGGTGGCTTGGCTTGGGGGCTTCCTGCATTCATTGGTTCAGCTCCTCCTGGTCCTTTGGT
TGCCCTTCTGTGGGCCCAATGTGATCAATCACTTTGCCTGTGACTTGTACCCTTTGCTGGAAGTTGCCTGCA
CCAATACGTATGTCATTGGTCTGCTGGTGGTTGCCAACAGTGGTTTAATCTGCCTGTTGAACTTCCTCATGC
TGGCTGCCTCCTACATTGTCATCCTGTACTCCTTGAGGTCCCACAGTGCAGATGGGAGATGCAAAGCCCTCT
CCACCTGTGGAGCCCACTTCATTGTTGTTGCCTTGTTCTTTGTGCCCTGTATATTTACTTATGTGCATCCAT
TTTCTACTTTACCTATAGACAAAAATATGGCATTATTTTATGGTATTCTGACACCTATGTTGAATCCACTCA
TTTATACCCTGAGAAATGAAGAGGTAAAAAATGCCATGAGAAAGCTCTTTACATGGTAAGAAATTGCAGG

In a search of public sequence databases, the NOV44 nucleic acid sequence, located on chromosome 7, has 783 of 920 bases (85%) identical to a gb:GENBANK-ID:AB030895|acc:AB030895.1 mRNA from Mus musculus (gene for odorant receptor MOR18, complete cds) (E=4.5e⁻¹⁴⁶).

The disclosed NOV44 polypeptide (SEQ ID NO:168) encoded by SEQ ID NO:167 has 302 amino acid residues and is presented in Table 44B using the one-letter amino acid code. Signal P, Psort and/or Hydropathy results predict that NOV44 has a signal peptide and is likely to be localized to the plasma membrane with a certainty of 0.6000. Alternatively, NOV44 may also localize to the Golgi body with a certainty of 0.4000, to the endoplasmic reticulum (membrane) with a certainty of 0.3000, or to the mitochondrial inner membrane with a certainty of 0.0300. The most likely cleavage site for NOV44 is between positions 39 and 40: VCG-NM.

TABLE 44B
Encoded NOV44 protein sequence.

(SEQ ID NO:168)

MDIPQNITEFFMLGLSQNSEVQRVLFVVFLLIYVVTVCGNMLIVVTITSSPTLASPVYFFLANLSFIDTFYS
SSMAPKLIADSLYEGRTISYECCMAQLFGAHFLGGVEIILLTVMAYDRYVAICKPLHNTTIMTRHLCAMLVG
VAWLGGFLHSLVQLLLVLWLPFCGPNVINHFACDLYPLLEVACTNTYVIGLLVVANSGLICLINFLMLAASY
IVILYSLRSHSADGRCKALSTCGAHFIVVALFFVPCIFTYVHPFSTLPIDKNMALFYGILTPMLNPLIYTLR
NEEVKNAMRKLFTW

A search of sequence databases reveals that the NOV44 amino acid sequence has 257 of 301 amino acid residues (85%) identical to, and 280 of 301 amino acid residues (93%) similar to, the 308 amino acid residue ptnr:SPTREMBL-ACC:Q9R0K2 protein from Mus musculus (Mouse) (Odorant Receptor MOR18) (E=5.0e⁻¹³⁸).

NOV44 also has homology to the amino acid sequences shown in the BLASTP data listed in Table 44C.

TABLE 44C
BLAST results for NOV44

| Gene Index/ | | Length | Identity | Positives | |
|---|---|---|---|---|---|
| Identifier | Protein/Organism | (aa) | (%) | (%) | Expect |
| gi\|17472367\|ref\|XP_{—} | similar to | 324 | 302/302 | 302/302 | e−143 |
| 061659.1\| | odorant receptor | | (100%) | (100%) | |
| (XM_061659) | 16 (H. sapiens) | | | | |
| | [Homo sapiens] | | | | |
| gi\|11496249\|ref\|NP_{—} | odorant receptor | 308 | 257/301 | 280/301 | e−127 |
| 067343.1\| | 16 [Mus musculus] | | (85%) | (92%) | |
| (NM_021368) | | | | | |
| gi\|11464995\|ref\|NP_{—} | gene for odorant | 302 | 234/300 | 262/300 | e−111 |
| 065261.1\| | receptor A16 [Mus | | (78%) | (87%) | |
| (NM_020515) | musculus] | | | | |
| gi\|17459946\|ref\|XP_{—} | similar to | 316 | 191/295 | 232/295 | 9e−95 |
| 062088.1\| | odorant receptor | | (64%) | (77%) | |
| (XM_062088) | 16 (H. sapiens) | | | | |
| | [Homo sapiens] | | | | |
| gi\|17472365\|ref\|XP_{—} | similar to | 544 | 183/296 | 230/296 | 1e−91 |
| 061658.1\| | odorant receptor | | (61%) | (76%) | |
| (XM_061658) | 16 (H. sapiens) | | | | |
| | [Homo sapiens] | | | | |

The homology between these and other sequences is shown graphically in the ClustalW analysis shown in Table 44D. In the ClustalW alignment of the NOV44 protein, as well as all other ClustalW analyses herein, the black outlined amino acid residues indicate regions of conserved sequence (i.e., regions that may be required to preserve structural or functional properties), whereas non-highlighted amino acid residues are less conserved and can potentially be altered to a much broader extent without altering protein structure or function.

[image]

[image]

Table 44E lists the domain description from DOMAIN analysis results against NOV44. This indicates that the NOV44 sequence has properties similar to those of other proteins known to contain this domain.

TABLE 44E
Domain Analysis of NOV44

gnl|Pfam|pfam00001, 7tm_1, 7 transmembrane receptor (rhodopsin
family). (SEQ ID NO:810)
CD-Length = 254 residues, 100.0% aligned
Score = 82.0 bits (201), Expect = 4e −17

| | | | |
|---|---|---|---|
| NOV43: | 39 | GNMLIVVTITSSPTLASPVYFFLANLSFIDTFYSSSMAPKLIADSLYEGRTISYECCMAQ | 98 |
| | | \|\|+\|+++ \| + \| +\| \|\| \|\|+ \| + ++ \| + + \| | |
| Sbjct: | 1 | GNLLVILVILRTKKLRTPTNIFLLNLAVADLLFLLTLPPWALYYLVGGDWVFGDALCKLV | 60 |
| NOV43: | 99 | LFGAHFLGGVEIILLTVMAYDRYVAICKPLHNTTIMTRHLCAMLVGVAWLGGFLHSLVQL | 158 |
| | | \| \|+\|\|\| ++ \|\|\|+\|\| \|\| \| \| +\|+ + \|+ \| \|\| \| | |
| Sbjct: | 61 | GALFVVNGYASILLLTAISIDRYLAIVHPLRYRRIRTPRRAKVLILLVWVLALLLSLPPL | 120 |
| NOV43: | 159 | LLVLWLPFCGPNVINHFACDLYPLLEVACTNTYVIGLLVVANSGLICLLNFLMLAASYIV | 218 |
| | | \| \| ++ + + \|+ ++ ++ | |
| Sbjct: | 121 | LFSWLRTVEEGNTTVCLIDFPEESVKRSYVLLSTLVGFVLPLLVILVCYTRILRTLRKRA | 180 |
| NOV43: | 219 | ILYSLRSHSADGRCKALSTCGAHFIVVALFFVPC-IFTYVHPF------STLPIDKNMAL | 271 |
| | | + \|\| +\| \| ++\| \| + \|\| + \| | |
| Sbjct: | 181 | RSQRSLKRRSSSERKAAKMLLVVVVVFVLCWLPYHIVLLLDSLCLLSIWRVLPTALLITL | 240 |
| NOV43: | 272 | FYGILTPMLNPLIY 285 | |
| | | + + \|\|\|+\|\| | |
| Sbjct: | 241 | WLAYVNSCLNPIIY 254 | |

G-protein-coupled receptors (GPCRs) constitute a vast protein family that encompasses a wide range of functions (including various autocrine, paracrine and endocrine processes). They show considerable diversity at the sequence level, on the basis of which they can be separated into distinct groups. We use the term clan to describe the GPCRs, as they embrace a group of families for which there are indications of evolutionary relationship, but between which there is no statistically significant similarity in sequence. The currently known clan members include the rhodopsin-like GPCRs, the secretin-like GPCRs, the cAMP receptors, the fungal mating pheromone receptors, and the metabotropic glutamate receptor family.

The rhodopsin-like GPCRs themselves represent a widespread protein family that includes hormone, neurotransmitter and light receptors, all of which transduce extracellular signals through interaction with guanine nucleotide-binding (G) proteins. Although their activating ligands vary widely in structure and character, the amino acid sequences of the receptors are very similar and are believed to adopt a common structural framework comprising 7 transmembrane (TM) helices

The disclosed NOV44 nucleic acid of the invention encoding a G Protein Coupled Receptor-like protein includes the nucleic acid whose sequence is provided in Table 44A or a fragment thereof. The invention also includes a mutant or variant nucleic acid any of whose bases may be changed from the corresponding base shown in Table 44A while still encoding a protein that maintains its UDP[Glycosyltransferase-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 15 percent of the bases may be so changed.

The disclosed NOV44 protein of the invention includes the G Protein Coupled Receptor-like protein whose sequence is provided in Table 44B. The invention also includes a mutant or variant protein any of whose residues may be changed from the corresponding residue shown in Table 44B while still encoding a protein that maintains its G Protein Coupled Receptor-like activities and physiological functions, or a functional fragment thereof. In the mutant or variant protein, up to about 39 percent of the residues may be so changed.

The invention further encompasses antibodies and antibody fragments, such as F_{ab} or (F_{ab})₂, that bind immunospecifically to any of the proteins of the invention.

The above disclosed information suggests that this G Protein Coupled Receptor-like protein (NOV44) is a member of a “G Protein Coupled Receptor family”. Therefore, the NOV44 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV44 nucleic acids and proteins of the invention are useful in potential therapeutic applications implicated in Systemic lupus erythematosus, Autoimmune disease, Asthma, Emphysema, Scleroderma, allergy, ARDS, and/or other diseases and pathologies.

NOV44 nucleic acids and polypeptides are further useful in the generation of antibodies that bind immuno-specifically to the novel NOV44 substances for use in therapeutic or diagnostic methods. These antibodies may be generated according to methods known in the art, using prediction from hydrophobicity charts, as described in the “Anti-NOVX Antibodies” section below. The disclosed NOV44 protein has multiple hydrophilic regions, each of which can be used as an immunogen. These novel proteins can be used in assay systems for functional analysis of various human disorders, which will help in understanding of pathology of the disease and development of new drug targets for various disorders.

NOV45

A disclosed NOV45 nucleic acid of 994 nucleotides (also referred to as CG56694-01) encoding a Mas Proto-Oncogene-like protein is shown in Table 45A. An open reading frame was identified beginning with a ATG initiation codon at nucleotides 17-19 and ending with a TGA codon at nucleotides 980-982. The start and stop codons are shown in bold in Table 45A, and the 5′ and 3′ untranslated regions, if any, are underlined.

TABLE 45A
NOV45 nucleotide sequence.

(SEQ ID NO:169)

ACTAGGGTTCCTGAGCATGATCCAACCATCCCAGCCTTGGGTACAGAACTGACACCAATCAATGGACGGGA
GGAGACTCCTTGCTACAAGCAAACCCTGAGCCTCACAGGGCTGACGTGCATCGTTTCCCTTGTCGGGATGAC
AGGAAATGCAGTCGTGCTCTGGCTCCTGGGCTTCCGCATGCGCAGGAACGCCTTCTCCATCTACATCTTCAA
CCTGTCCATGGCCGACTTCCTCTTTCTCAGAAGCCACATTATACGTTTTCCGTTAAGCCTCATCAATATCCT
CCATCCCATCTTCAAAATCCTCAGCCCTGTGATGATGTTTTCCTACCTTGCAAGCCTGAGCTTTCTAAGCGC
CATGAGCACCGAGCGCTGCCTGTACGTCCTGTGGCCCATCTGGGAGCGCTGCCGCCCCCGCCCCTACACCTG
TCAGCGGTCGTGTGTGTCATGCTCTGGGCCCTGTCTCTGCTGCGGAGCGTCCTGGAGTGGAGTTTCTGTGAC
TTCCTGTTTAGTGGTGCTGATTCTGTTTGGTGTAAAACATCAGATTTCATCATAGTAGGGGGGCTGATTTTT
TTATGTGTGGCTCTCTGTGGTTCCAGCCTGGTCCTGCTGGTCAGGATCCTTTGTGGGTCCCGGAAGATGCCA
CTGACCAGGCTGTACGTGACCATCCTGCTCATAGCGCTGGTCTTCCTCCTCTGTGGCCTGCCCTTTGGCATT
CGGTTTTTCCTATTTTCATGGAACCACGTGGATTTGGAAGTCTTATATTGTCACGTTCATCTAGTTTCCATT
TTCCTTTCCTCTCTTAACGGCCAACCCCAACATTTACTTCTTCGTGGGCTCCTTAAGGCAGTGTCAAAAAAG
GCAGAACCTGAAGCTGGTTCTCCAGAGGGCTCTGCAGGACACGACTGAGGTGAATGAAGGTGGACGATGGCT
TCCTGAGGAAACCCTGGAGCTGTCAGGAAGCAGATTTGGGCAGTGAGGAAGAGCCTCT

In a search of public sequence databases, the NOV45 nucleic acid sequence, located on chromosome 7, has 353 of 580 bases (60%) identical to a gb:GENBANK-ID:I08606|acc:I08606.1 mRNA from Unknown. (Sequence 1 from Patent WO 8707472) (E=1.2e⁻¹⁰).

The disclosed NOV45 polypeptide (SEQ ID NO:170) encoded by SEQ ID NO:169 has 319 amino acid residues and is presented in Table 45B using the one-letter amino acid code. Signal P, Psort and/or Hydropathy results predict that NOV45 has no signal peptide and is likely to be localized to the plasma membrane with a certainty of 0.6400. Alternatively, NOV45 may also localize to the Golgi body with a certainty of 0.4600, to the endoplasmic reticulum (membrane) with a certainty of 0.3700, or to the endoplasmic reticulum (lumen) with a certainty of 0.1000.

TABLE 45B
Encoded NOV45 protein sequence.

(SEQ ID NO:170)

MIQLLITSGLCSQGHQTRVPEHGSNHPSLGYRTDTNQWTGGDSLLQANPEPHRADVHRFPCRDDRKCSRALA
PGLPHAQERLLHLHLQPVHGRLPLSQKPHYTFSVKPHQYPPSHLQNPQPCDDVELPCKPELSKRHEHRALPV
RPVAHLGALPPPPLHLSAVVCVMLWALSLLRSVLEWSFCDFLFSGADSVWCKTSDFIIVGGLIFLCVALCGS
SLVLLVRILCGSRKMPLTRLYVTILLIALVFLLCGLPFGIRFFLFSWNHVDLEVLYCHVHLVSIFLSSLNGQ
PQHLLLRGLLKAVSKKAEPEAGSPEGSAGHD

A search of sequence databases reveals that the NOV45 amino acid sequence has 50 of 168 amino acid residues (29%) identical to, and 87 of 168 amino acid residues (51%) similar to, the 378 amino acid residue ptnr:SWISSPROT-ACC:P35410 protein from Homo sapiens (Human) (Mas-Related G Protein-Coupled Receptor MRG) (E=5.0e−138).

NOV45 also has homology to the amino acid sequences shown in the BLASTP data listed in Table 45C.

TABLE 45C
BLAST results for NOV45

| Gene Index/ | | Length | Identity | Positives | |
|---|---|---|---|---|---|
| Identifier | Protein/Organism | (aa) | (%) | (%) | Expect |
| gi\|15546062\|gb\|AAK91804.1\| | MrgX1 G protein- | 322 | 108/130 | 116/130 | 2e−56 |
| (AY042213) | coupled receptor | | (83%) | (89%) | |
| | [Homo sapiens] | | | | |
| gi\|17472340\|ref\|XP_{—} | similar to MrgX1 | 1589 | 108/130 | 116/130 | 3e−56 |
| 061650.1\| | G protein-coupled | | (83%) | (89%) | |
| (XM_061650) | receptor (H. | | | | |
| | sapiens) [Homo | | | | |
| | sapiens] | | | | |
| gi\|16876453\|ref\|NP_{—} | G protein-coupled | 322 | 104/130 | 118/130 | 2e−53 |
| 473372.1\| | receptor MRGX3 | | (80%) | (90%) | |
| (NM_054031) | [Homo sapiens] | | | | |
| gi\|17461239\|ref\|XP_{—} | similar to MrgX3 | 322 | 103/130 | 118/130 | 7e−53 |
| 062249.1\| | G protein-coupled | | (79%) | (90%) | |
| (XM_062249) | receptor (H. | | | | |
| | sapiens) [Homo | | | | |
| | sapiens] | | | | |
| gi\|16876455\|ref\|NP_{—} | G protein-coupled | 322 | 97/124 | 104/124 | 2e−46 |
| 473373.1\| | receptor MRGX4 | | (78%) | (83%) | |
| (NM_054032) | [Homo sapiens] | | | | |

The homology between these and other sequences is shown graphically in the ClustalW analysis shown in Table 45D. In the ClustalW alignment of the NOV45 protein, as well as all other ClustalW analyses herein, the black outlined amino acid residues indicate regions of conserved sequence (i.e., regions that may be required to preserve structural or functional properties), whereas non-highlighted amino acid residues are less conserved and can potentially be altered to a much broader extent without altering protein structure or function.

[image]

[image]

[image]

[image]

[image]

Table 45E lists the domain description from DOMAIN analysis results against NOV45. This indicates that the NOV45 sequence has properties similar to those of other proteins known to contain this domain.

TABLE 45E
Domain Analysis of NOV45

gnl|Pfam|pfam00001, 7tm_1, 7 transmembrane receptor (rhodopsin
family). (SEQ ID NO:810)
CD-Length = 41.2 bits (95), Expect = 9e−05

| | | | |
|---|---|---|---|
| NOV44: | 165 | CVMLWALSLLRSVLE-------------WSFCDFLFSGADSVWCKTSDFIIVGGLIFLCV | 211 |
| | | +++\| \|+\|\| \|+ + \| \| +\|\| ++ \| \| | 211 |
| Sbjct: | 105 | ILLVWVLALLLSLPPLLFSWLRTVEEGNTTVCLIDFPEESVKRSYVLLSTLVGFVLPLLV | |
| NOV44: | 212 | ALCGSSLVLL----VRILCGSRKMPL---TRLYVTILLIALVFLLCGLPFGIRFFLFSWN | 264 |
| | | \| + +\| \| \| + +\|++ +\|\|+\|\| \|\|+ \| \| \| | |
| Sbjct: | 165 | ILVCYTRILRTLRKRARSQRSLKRRSSSERKAAKMLLVVVVVFVLCWLPYHIVLLIDSLC | 224 |
| NOV44: | 265 | HVDLEVLYCHVHLVSIFLSSLN 286 | |
| | | + + + \|++++\|+ +\| | |
| Sbjct: | 225 | LLSIWRVLPTALLITLWLAYVN 246 | |

The Mas Proto-Oncogene belongs to the family of G-Protein Coupled Receptors.G-protein-coupled receptors (GPCRs) constitute a vast protein family that encompasses a wide range of functions (including various autocrine, paracrine and endocrine processes). They show considerable diversity at the sequence level, on the basis of which they can be separated into distinct groups. We use the term clan to describe the GPCRs, as they embrace a group of families for which there are indications of evolutionary relationship, but between which there is no statistically significant similarity in sequence [1]. The currently known clan members include the rhodopsin-like GPCRs, the secretin-like GPCRs, the cAMP receptors, the fungal mating pheromone receptors, and the metabotropic glutamate receptor family.

The human mas oncogene was originally detected by its ability to transform NIH 3T3 cells. We previously showed that the protein encoded by this gene is unique among cellular oncogene products in that it has seven hydrophobic potential transmembrane domains and shares strong sequence similarity with a family of hormone-receptor proteins. We have now cloned the rat homolog of the mas oncogene, determined its DNA sequence, and examined its expression in various rat tissues. A comparison of the predicted sequences of the rat and human mas proteins shows that they are highly conserved, except in their hydrophilic amino-terminal domains. Our examination of the expression of mas, determined by RNA-protection studies, indicates that high levels of mas RNA transcripts are present in the hippocampus and cerebral cortex of the brain, but not in other neural regions or in other tissues. This pattern of expression and the similarity of mas protein to known receptor proteins suggest that mas encodes a receptor that is involved in the normal neurophysiology and/or development of specific neural tissues.

The rhodopsin-like GPCRs themselves represent a widespread protein family that includes hormone, neurotransmitter and light receptors, all of which transduce extracellular signals through interaction with guanine nucleotide-binding (G) proteins. Although their activating ligands vary widely in structure and character, the amino acid sequences of the receptors are very similar and are believed to adopt a common structural framework comprising 7 transmembrane (TM) helices

The disclosed NOV45 nucleic acid of the invention encoding a Mas Proto-Oncogene-like protein includes the nucleic acid whose sequence is provided in Table 45A or a fragment thereof. The invention also includes a mutant or variant nucleic acid any of whose bases may be changed from the corresponding base shown in Table 45A while still encoding a protein that maintains its Mas Proto-Oncogene-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 40 percent of the bases may be so changed.

The disclosed NOV45 protein of the invention includes the Mas Proto-Oncogene-like protein whose sequence is provided in Table 45B. The invention also includes a mutant or variant protein any of whose residues may be changed from the corresponding residue shown in Table 45B while still encoding a protein that maintains its Mas Proto-Oncogene-like activities and physiological functions, or a functional fragment thereof. In the mutant or variant protein, up to about 32 percent of the residues may be so changed.

The invention further encompasses antibodies and antibody fragments, such as F_{ab} or (F_{ab})₂, that bind immunospecifically to any of the proteins of the invention.

The above disclosed information suggests that this Mas Proto-Oncogene-like protein (NOV45) is a member of a “Mas Proto-Oncogene family”. Therefore, the NOV45 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV45 nucleic acids and proteins of the invention are useful in potential therapeutic applications implicated in Von Hippel-Lindau (VHL) syndrome, Alzheimer's disease, Stroke, Tuberous sclerosis, hypercalceimia, Parkinson's disease, Huntington's disease, Cerebral palsy, Epilepsy, Lesch-Nyhan syndrome, Multiple sclerosis, Ataxia-telangiectasia, Leukodystrophies, Behavioral disorders, Addiction, Anxiety, Pain, Neuroprotection, and/or other diseases and pathologies.

NOV45 nucleic acids and polypeptides are further useful in the generation of antibodies that bind immuno-specifically to the novel NOV45 substances for use in therapeutic or diagnostic methods. These antibodies may be generated according to methods known in the art, using prediction from hydrophobicity charts, as described in the “Anti-NOVX Antibodies” section below. The disclosed NOV45 protein has multiple hydrophilic regions, each of which can be used as an immunogen. These novel proteins can be used in assay systems for functional analysis of various human disorders, which will help in understanding of pathology of the disease and development of new drug targets for various disorders.

NOV46

NOV46 includes three novel Mas Proto-Oncogene-like proteins disclosed below. The disclosed sequences have been named NOV46a and NOV46b.

NOV46a

A disclosed NOV46a nucleic acid of 997 nucleotides (also referred to as CG56696-01) encoding a Mas Proto-Oncogene-like protein is shown in Table 46A. An open reading frame was identified beginning with a ATG initiation codon at nucleotides 12-14 and ending with a TGA codon at nucleotides 978-980. The start and stop codons are shown in bold in Table 46A, and the 5′ and 3′ untranslated regions, if any, are underlined.

TABLE 46A
NOV46a nucleotide sequence.

(SEQ ID NO:171)

GGTTTCTGAGCATGGATCCAACCATCTCAACCTTGGACACAGAACTGACACCAATCAACGGAACTGAGGAGA
CTCTTTGCTACAAGCAGACCTTGAGCCTCACGGTGCTGACGTGCATCGTTTCCCTTGTCGGGCTGACAGGAA
ACGCAGTTGTGCTCTGGCTCCTGGGCTGCCGCATGCGCAGGAACGCCTTCTCCATCTACATCCTCAACTTGG
CCGCAGCAGACTTCCTCTTCCTCAGCGGCCGCCTTATATATTCCCTGTTAAGCTTCATCAGTATCCCCCATA
CCATCTCTAAAATCCTCTATCCTGTGATGATGTTTTCCTACTTTGCAGGCCTGAGCTTTCTGAGTGCCGTGA
GCACCGAGCGCTGCCTGTCCGTCCTGTGGCCCATCTGGTACCGCTGCCACCGCCCCACACACCTGTCAGCGG
TGGTGTGTGTCCTGCTCTGGGCCCTGTCCCTGCTGCGGAGCATCCTGGAGTGGATGTTATGTGGCTTCCTGT
TCAGTGGTGCTGATTCTGCTTGGTGTCAAACATCAGATTTCATCACAGTCGCGTGGCTGATTTTTTTATGTG
TGGTTCTCTGTGGGTCCAGCCTGGTCCTGCTGATCAGGATTCTCTGTGGATCCCGGAAGATACCGCTGACCA
GGCTGTACGTGACCATCCTGCTCACAGTACTGGTCTTCCTCCTCTGTGGCCTGCCCTTTGGCATTCAGTTTT
TCCTATTTTTATGGATCCACGTGGACAGGGAAGTCTTATTTTGTCATGTTCATCTAGTTTCTATTTTCCTGT
CCGCTCTTAACAGCAGTGCCAACCCCATCATTTACTTCTTCGTGGGCTCCTTTAGGCAGCGTCAAAATAGGC
AGAACCTGAAGCTGGTTCTCCAGAGGGCTCTGCAGGACGCGTCTGAGGTGGATGAAGGTGGAGGGCAGCTTC
CTGAGGAAATCCTGGAGCTGTCGGGAAGCAGATTGGAGCAGTGAGGAAGAGCCTCTGCCCT

In a search of public sequence databases, the NOV46a nucleic acid sequence, located on chromosome 7, has 430 of 705 bases (60%) identical to a gb:GENBANK-ID:MMU249895|acc:AJ249895.1 mRNA from Mus musculus (mas proto-oncogene and Igf2r gene for insulin-like growth factor type 2 and L41ps and Au76 pseudogenes) (E=9.3e⁻²²).

The disclosed NOV46a polypeptide (SEQ ID NO:172) encoded by SEQ ID NO:171 has 322 amino acid residues and is presented in Table 46B using the one-letter amino acid code. Signal P, Psort and/or Hydropathy results predict that NOV46a has a signal peptide and is likely to be localized to the plasma membrane with a certainty of 0.6000. Alternatively, NOV46a may also localize to the Golgi body with a certainty of 0.4000, to the endoplasmic reticulum (membrane) with a certainty of 0.3000, or to the microbody (peroxisome) with a certainty of 0.3000. The most likely cleavage site for NOV46a is between positions 45 and 46: TGN-AV.

TABLE 46B
Encoded NOV46a protein sequence.

(SEQ ID NO:172)

MDPTISTLDTELTPINGTEETLCYKQTLSLTVLTCIVSLVGLTGNAVVLWLLGCRMRRNAFSIYILNLAAAD
FLFLSGRLIYSLLSFISIPHTISKILYPVMMFSYFAGLSFLSAVSTERCLSVLWPIWYRCHRPTHLSAVVCV
LLWALSLLRSILEWMLCGFLFSGADSAWCQTSDFITVAWLIFLCVVLCGSSLVLLIRILCGSRKIPLTRLYV
TILLTVLVFLLCGLPFGIQFFLFLWIHVDREVLFCHVHLVSIFLSALNSSANPIIYFFVGSFRQRQNRQNLK
LVLQRALQDASEVDEGGGQLPEEILELSGSRLEQ

A search of sequence databases reveals that the NOV46a amino acid sequence has 110 of 275 amino acid residues (40%) identical to, and 175 of 275 amino acid residues (63%) similar to, the 324 amino acid residue ptnr:SWISSPROT-ACC:P12526 protein from Rattus norvegicus (Rat) (Mas Proto-Oncogene) (E=1.6e⁻⁴⁵).

NOV46b

In the present invention, the target sequence identified previously, NOV46a, was subjected to the exon linking process to confirm the sequence. PCR primers were designed by starting at the most upstream sequence available, for the forward primer, and at the most downstream sequence available for the reverse primer. In each case, the sequence was examined, walking inward from the respective termini toward the coding sequence, until a suitable sequence that is either unique or highly selective was encountered, or, in the case of the reverse primer, until the stop codon was reached. Such primers were designed based on in silico predictions for the full length cDNA, part (one or more exons) of the DNA or protein sequence of the target sequence, or by translated homology of the predicted exons to closely related human sequences sequences from other species. These primers were then employed in PCR amplification based on the following pool of human cDNAs: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea, uterus. Usually the resulting amplicons were gel purified, cloned and sequenced to high redundancy. The resulting sequences from all clones were assembled with themselves, with other fragments in CuraGen Corporation's database and with public ESTs. Fragments and ESTs were included as components for an assembly when the extent of their identity with another component of the assembly was at least 95% over 50 bp. In addition, sequence traces were evaluated manually and edited for corrections if appropriate. These procedures provide the sequence reported below, which is designated NOV46b. This differs from the previously identified sequence (NOV46a) in having 2 amino acid changes.

A disclosed NOV46b nucleic acid of 964 nucleotides (also referred to as CG56696-02) encoding a Mas-Related G Protein-Coupled Receptor-like protein is shown in Table 46C. An open reading frame was identified beginning with a ACC initiation codon at nucleotides 3-5 and ending with a TGA codon at nucleotides 960-962. The start and stop codons are shown in bold in Table 46C, and the 5′ and 3′ untranslated regions, if any, are underlined. Because the start codon is not a traditional initiation codon, NOV46b could be a partial reading frame extending further in the 5′ direction.

TABLE 46C
NOV46b nucleotide sequence.

(SEQ ID NO:173)

CAACCATCTCAACCTTGGACACAGAACTGACACCAATCAACGGAACTGAGGAGACTCTTTGCTACAAGCAGA
CCTTGAGCCTCACGGTGCTGACGTGCATCGTTTCCCTTGTCGGGCTGACAGGAAACGCGGTTGTGCTCTGGC
TCCTGGGCTGCCGCATGCGCAGGAACGCCTTCTCCATCTACATCCTCAACTTGGCCGCAGCAGACTTCCTCT
TCCTCAGCGGCCGCCTTATATATTCCCTGTTAAGCTTCATCAGTATCCCCCATACCATCTCTAAAATCCTCT
ATCCTGTGATGATGTTTTCCTACTTTGCAGGCCTGAGCTCTCTGAGTGCCGTGAGCACCGAGCGCTGCCTGT
CCGTCCTGTGGCCCATCTGGTACCGCTGCCACCGCCCCACACACCTGTCAGCGGTGGTGTGTGTCCTGCTCT
GGGCCCTGTCCCTGCTGCGGAGCATCCTGGAGTGGATGTTATGTGGCTTTCTGTTCAGTGGTGCTGATTCTG
CTTGGTGTCAAACATCAGATTTCATCACAGTCGCGTGGCTGATTTTTTTATGTGTGGTTCTCTGTGGGTCCA
GCCTGGTCCTGCTGATCAGGATTCTCTGTGGATCCCGGAAGATACCGCTGACCAGGCTGTACGTGACCATCC
CGCTCACAGTACTGGTCTTCCTCCTCTGTGGCCTGCCCTTTGGCATTCAGTTTTTCCTATTTTTATGGATCC
ACGTGGACAGGGAAGTCTTATTTTGTCATGTTCATCTAGTTTCTATTTTCCTGTCCGCTCTTAACAGCAGTG
CCAACCCCATCATTTACTTCTTCGTGGGCTCCTTTAGGCAGCGTCAAAATAGGCAGAACCTGAAGCTGGTTC
TCCAGAGGGCTCTGCAGGACGCGTCTGAGGTGGATGAAGGTGGAGGGCAGCTTCCTGAGGAAATCCTGGAGC
TGTCGGGAAGCAGATTGGAGCAGTGAGG

In a search of public sequence databases, the NOV46b nucleic acid sequence, located on chromosome 11, has 494 of 800 bases (61%) identical to a gb:GENBANK-ID:AF295365|acc:AF295365.1 mRNA from Mus musculus (G-protein coupled receptor GPR90 mRNA, complete cds) (E=1.2e⁻²⁸).

The disclosed NOV46b polypeptide (SEQ ID NO:174) encoded by SEQ ID NO:173 has 319 amino acid residues and is presented in Table 46D using the one-letter amino acid code. Signal P, Psort and/or Hydropathy results predict that NOV46b has a signal peptide and is likely to be localized to the plasma membrane with a certainty of 0.6000. Alternatively, NOV46b may also localize to the Golgi body with a certainty of 0.4000, to the endoplasmic reticulum (membrane) with a certainty of 0.3000, or to the microbody (peroxisome) with a certainty of 0.3000. The most likely cleavage site for NOV46b is between positions 42 and 43: TGN-AV.

TABLE 46D
Encoded NOV46b protein sequence.

(SEQ ID NO:174)

TISTLDTELTPINGTEETLCYKQTLSLTVLTCIVSLVGLTGNAVVLWLLGCRMRRNAFSIYILNLAAADFLF
LSGRLIYSLLSFISIPHTISKILYPVMMFSYFAGLSSLSAVSTERCLSVLWPIWYRCHRPTHLSAVVCVLLW
ALSLLRSILEWMLCGFLFSGADSAWCQTSDFITVAWLIFLCVVLCGSSLVLLIRILCGSRKIPLTRLYVTIP
LTVLVFLLCGLPFGIQFFLFLWIHVDREVLFCHVHLVSIFLSALNSSANPIIYFFVGSFRQRQNRQNLKLVL
QRALQDASEVDEGGGQLPEEILELSGSRLEQ

A search of sequence databases reveals that the NOV46b amino acid sequence has 110 of 275 amino acid residues (40%) identical to, and 174 of 275 amino acid residues (63%) similar to, the 324 amino acid residue ptnr:SWISSPROT-ACC:P12526 protein from Rattus norvegicus (Rat) (Mas Proto-Oncogene) (E=6.8e⁻⁴⁵).

NOV46c

A disclosed NOV46c nucleic acid of 1030 nucleotides (also referred to as CG56698-01) encoding a Mas Proto-Oncogene-like protein is shown in Table 46E. An open reading frame was identified beginning with a ATG initiation codon at nucleotides 17-19 and ending with a TGA codon at nucleotides 1007-1009. The start and stop codons are shown in bold in Table 46E, and the 5′ and 3′ untranslated regions, if any, are underlined.

TABLE 46E
NOV46c nucleotide sequence.

(SEQ ID NO:175)

ACTAGGGTTTCTGAGCATGAATCCAACCATCCCAGCCTTGGATACAGAAATTGCACCAATTAGTGATACAGA
GGAGACCCATCCTCATCGTTGTGGCATGGAGGTCCTGGTCCTCATAGTGCTGATCCTCATCATTGACCTGGT
CGGGCTGGCAGGAAATGCAGTCATGCTCTGGCTCCTGGGCTTCTGCATGCACAGTAACACCTTCTCTCTCTA
CATCCTCAACCTGGCCAGGGCTGACTTCCTCTGCACCTGCTTCCAGATTATAACATTCATTAATTTCTTCAG
TGACTTTGTTAGTTCTCTCTCCATCCATTTCTCTAGATTTGTCACCACGGTGTTGTTCTCCGCCTGTATTAC
AGGCCTGAGCATGCTGAGCACCATCAGCACCGAGCACCGCCTGTCCGTCCTGTGGCCCATCTGGTACTGCTG
CCACTGCCCCACACACCTGTCAGCGGTCATGTGTGTCCTGCTCTGGGCCCTGTCCCTGTTGCAGAGCATCCT
GGAGTGGATGTTCTGTAGCTTCCTGTTTAGTGATGTTGACTCTGATAATTGGTGTCAAATATTAGATTTCCT
CACTGCTGTGTGGCTGATTTTTTTATCTGTGGTTCTCTGTGGGTTCACCCTGGTCCTGCTTGTCAGGATCAT
ATGTGGATCCCAGAAGATGCCGCTGACCAGGCTGTATGTGACCATCCTGCTCACAGGGCTGGTCTTCCTCTT
CTGCAGCCTGCCCCTCAGCATTCAGGGATTCCTATTATACTGGATCGAGAAGGATTTGGATGACTTACCTTG
TGTTGTTCGTTTAATTTCCATTTTCCTGTCTGCTCTTAACAGCAGTGCCAACCCCATCATTTACTTCTTCAT
GGGCTCCTTTAGGCAGCTTCAAAACAGGAAGACCCTCAAGCTGGTTCTCCAGAGGGCTCTGCAGGACATGCT
TGAGGTGGATGAAGGTGGAGGGCAGCTTCCTGAGGAAACCCTGAAGCTGTCGGGAAGCAGATTGGGGCCATG
AGGAAGAGCCTCTGCCCTGTTA

In a search of public sequence databases, the NOV46c nucleic acid sequence, located on chromosome 7, has 381 of 621 bases (61%) identical to a gb:GENBANK-ID:RATMAS|acc:J03823.1 mRNA from Rattus norvegicus (Rat mas oncogene, complete cds) (E=9.3e⁻²²).

The disclosed NOV46D polypeptide (SEQ ID NO:176) encoded by SEQ ID NO:175 has 330 amino acid residues and is presented in Table 46F using the one-letter amino acid code. Signal P, Psort and/or Hydropathy results predict that NOV46c has a signal peptide and is likely to be localized to the plasma membrane with a certainty of 0.6000. Alternatively, NOV46c may also localize to the Golgi body with a certainty of 0.4000, to the endoplasmic reticulum (membrane) with a certainty of 0.3000, or to the microbody (peroxisome) with a certainty of 0.3000. The most likely cleavage site for NOV46c is between positions 65 and 66: TFS-LY.

TABLE 46F
Encoded NOV46c protein sequence.

(SEQ ID NO:176)

MNPTIPALDTEIAPISDTEETHPHRCGMEVLVLIVLILIIDLVGLAGNAVMLWLLGFCMHSNTFSLYILNLA
RADFLCTCFQIITFINFFSDFVSSLSIHFSRFVTTVLFSACITGLSMLSTISTEHRLSVLWPIWYCCHCPTH
LSAVMCVLLWALSLLQSILEWMFCSFLFSDVDSDNWCQILDFLTAVWLIFLSVVLCGFTLVLLVRIICGSQK
MPLTRLYVTILLTGLVFLFCSLPLSIQGFLLYWIEKDLDDLPCVVRLISIFLSALNSSANPIIYFFMGSFRQ
LQNRKTLKLVLQRALQDMLEVDEGGGQLPEETLKLSGSRLGP

A search of sequence databases reveals that the NOV46c amino acid sequence has 106 of 279 amino acid residues (37%) identical to, and 166 of 279 amino acid residues (59%) similar to, the 324 amino acid residue ptnr:SWISSPROT-ACC:P12526 protein from Rattus norvegicus (Rat) (Mas Proto-Oncogene) (E=1.6e⁻⁴⁵).

NOV46c is predicted to be expressed in at least teratocarcinoma cell. This information was derived by determining the tissue sources of the sequences that were included in the invention including but not limited to SeqCalling sources, Public EST sources, Literature sources, and/or RACE sources.

In addition, the sequence is predicted to be expressed in hippocampus and brain because of the expression pattern of (GENBANK-ID: gb:GENBANK-ID:RATMAS|acc:J03823.1) a closely related Rat mas oncogene, complete cds homolog.

NOV46d

A disclosed NOV46d nucleic acid of 1005 nucleotides (also referred to as CG56702-01) encoding a Mas Proto-Oncogene-like protein is shown in Table 46G. An open reading frame was identified beginning with a ATG initiation codon at nucleotides 17-19 and ending with a TGA codon at nucleotides 986-988. The start and stop codons are shown in bold in Table 46G, and the 5′ and 3′ untranslated regions, if any, are underlined.

TABLE 46G
NOV46d nucleotide sequence.

(SEQ ID NO:177)

ACTACCGTTTCTGAGCATGGATCCAAGCAACCCAGCCTTGGATGCAGAACTGACACCAATTAACAGAACTGA
GGAGACTCCTTGCTACAAGCAGACCCTGAGCCTCATGGGGCTGACGTGCATCATTTCCCTTGTCACGCTGAC
AGGAAACGCGGTTGTGCTCTGGCTCCTGGGCTTCCGCATGCGCAGGAACGCCGTCTCCATCTACATCCTCAA
CCTGGCTGCGGCAGACTTCCTCTTCCTCAGCGGCCACGTTATACATTCCGCCTCACTCCTCATCAATATCTG
TCATCCCATCTCCAAAATCCTCATTCCTGTGATGACCTTTCTATACTTTACAGGCCTGAGCTTTCTGAGTGC
CATGAGCACCGAGCGCTGCCTGTGCGTCCTGTGGCCCATCTGGTACCGCTGCCTCCTCCCCACACACCTGTC
AGCGGTCGTGTGTGTCTTGCTTTGGGCCCTGTCCCTACTGCGGAGCATCCTGGAGGGAATGTTCTGTGACTT
CCTGTTTAGTGATGCTGATTCTATTTGGTGTCAACCATCAGATTTCATCACAGTCGTGTGGCTGATTTTTTT
ATGTGTGGTTCTCTGTGGGTCCAGCCTGGTCCTGCTGATTAGGATTCTCTGTGGATCCTGGAAGATGCCTCT
GACCGGGCTGTACGTGACGATCCTGCTCACAGTGCTAGTCTTCCTACTCCGCAGCCTGCCCTTCGGCATTCG
GTGGGCTCTGTCTACTGGGATCCACCTGGATTTGGAAGTCATTTTCTGTCATGTCCATCTAGTTTCCATTTT
CCTGTCCCCTCTAAACGGTAGTGCCAACCCCGTCATTTACTTCTTCGTGGGCTCCTTTAGGCAGCGTCAAAA
TAGGCAGAACCTGAAGCTGGTTCTCCAGAGGGCTCTGCAGGACATGCCTGAGGTGAAGGTGGAAGGTGGAGG
GCGGCTTCCTGAGGGAACCCTGGAGCTGTCGGGAAGCAGATTCGGGCAGTGAGGAAGAACCTCTGCCCT

In a search of public sequence databases, the NOV46d nucleic acid sequence, located on chromosome 7, has 379 of 632 bases (59%) identical to a gb:GENBANK-ID:RATMAS|acc:J03823.1 mRNA from Rattus norvegicus (Rat mas oncogene, complete cds) (E=4.7e⁻¹⁴).

The disclosed NOV46d polypeptide (SEQ ID NO:178) encoded by SEQ ID NO:177 has 323 amino acid residues and is presented in Table 46H using the one-letter amino acid code. Signal P, Psort and/or Hydropathy results predict that NOV46C has a signal peptide and is likely to be localized to the plasma membrane with a certainty of 0.6000. Alternatively, NOV46d may also localize to the Golgi body with a certainty of 0.4000, to the endoplasmic reticulum (membrane) with a certainty of 0.3000, or to the microbody (peroxisome) with a certainty of 0.3000. The most likely cleavage site for NOV46d is between positions 46 and 47: GNA-VV.

TABLE 46H
Encoded NOV46d protein sequence.

(SEQ ID NO:178)

MDPSNPALDAELTPINRTEETPCYKQTLSLMGLTCIISLVTLTGNAVVLWLLGFRMRRNAVSIYILNLAAAD
FLFLSGHVIHSASLLINICHPISKILIPVMTFLYFTGLSFLSAMSTERCLCVLWPIWYRCLLPTHLSAVVCV
LLWALSLLRSILEGMFCDFLFSDADSIWCQPSDFITVVWLIFLCVVLCGSSLVLLIRILCGSWKMPLTGLYV
TILLTVLVFLLRSLPFGIRWALSTGIHLDLEVIFCHVHLVSIFLSPLNGSANPVIYFFVGSFRQRQNRQNLK
LVLQRALQDMPEVKVEGGGRLPEGTLELSGSRFGQ

A search of sequence databases reveals that the NOV46d amino acid sequence has 107 of 275 amino acid residues (38%) identical to, and 167 of 275 amino acid residues (60%) similar to, the 324 amino acid residue ptnr:SWISSPROT-ACC:P12526 protein from Rattus norvegicus (Rat) (Mas Proto-Oncogene) (E=1.5e⁻⁴⁰).

In addition, NOV46d is predicted to be expressed in hippocampus and cerebral cortex of the brain because of the expression pattern of (GENBANK-ID: gb:GENBANK-ID:RATMAS|acc:J03823.1) a closely related Rat mas oncogene, complete cds homolog.

NOV46a also has homology to the amino acid sequences shown in the BLASTP data listed in Table 46I.

TABLE 46I
BLAST results for NOV46a

| Gene Index/ | | Length | Identity | Positives | |
|---|---|---|---|---|---|
| Identifier | Protein/Organism | (aa) | (%) | (%) | Expect |
| gi\|17472340\|ref\|XP_{—} | similar to MrgX1 | 1589 | 322/322 | 322/322 | e−172 |
| 061650.1\| | G protein-coupled | | (100%) | (100%) | |
| (XM_061650) | receptor (H. | | | | |
| | sapiens) [Homo | | | | |
| | sapiens] | | | | |
| gi\|15546062\|gb\|AAK91804.1\| | MrgX1 G protein- | 322 | 322/322 | 322/322 | e−158 |
| (AY042213) | coupled receptor | | (100%) | (100%) | |
| | [Homo sapiens] | | | | |
| gi\|16876453\|ref\|NP_{—} | G protein-coupled | 322 | 269/322 | 285/322 | e−131 |
| 473372.1\| | receptor MRGX3 | | (83%) | (87%) | |
| (NM_054031) | [Homo sapiens] | | | | |
| gi\|17461239\|ref\|XP_{—} | similar to MrgX3 | 322 | 268/322 | 285/322 | e−131 |
| 062249.1\| | G protein-coupled | | (83%) | (88%) | |
| (XM_062249) | receptor (H. | | | | |
| | sapiens) [Homo | | | | |
| | sapiens] | | | | |
| gi\|16876455\|ref\|NP_{—} | G protein-coupled | 322 | 255/320 | 275/320 | e−119 |
| 473373.1\| | receptor MRGX4 | | (79%) | (85%) | |
| (NM_054032) | [Homo sapiens] | | | | |

The homology between these and other sequences is shown graphically in the ClustalW analysis shown in Table 46J. In the ClustalW alignment of the NOV46 protein, as well as all other ClustalW analyses herein, the black outlined amino acid residues indicate regions of conserved sequence (i.e., regions that may be required to preserve structural or functional properties), whereas non-highlighted amino acid residues are less conserved and can potentially be altered to a much broader extent without altering protein structure or function.

[image]

[image]

[image]

[image]

[image]

[image]

Table 46K lists the domain description from DOMAIN analysis results against NOV46. This indicates that the NOV46 sequence has properties similar to those of other proteins known to contain this domain.

TABLE 46K
Domain Analysis of NOV46

gnl|Pfam|pfam00001, 7tm_1, 7 transmembrane receptor (rhodopsin
family). (SEQ ID NO:810)
CD-Length = 254 residues, 37.8% aligned
Score =51.2 bits (121), Expect = 9e−08

| | | | |
|---|---|---|---|
| NOV53: | 44 | GNAVVLWLLGCRMR-RNAFSIYILNLAAADFLFLSGRLIYSLLSFIS----IPHTISKIL | 98 |
| | | \|\| +\|+ ++ + \| +\|++\|\|\|\| \|\| \|\|\| ++\| + + \|++ | |
| Sbjct: | 1 | GNLLVILVILRTKKLRTPTNIFLLNLAVADLLFLLTLPPWALYYLVGGDWVFGDALCKLV | 60 |
| NOV53: | 99 | YPVMMFSYFAGLSFLSAVSTERCLSVLWPIWYRCHR 134 | |
| | | + + + +\| + \|+\|+\| +\| \|+++ \|+ \|\| \| | |
| Sbjct: | 61 | GALFVVNGYASILLLTAISIDRYLAIVHPLRYRRIR 96 | |

The Mas Proto Oncogene belongs to the family of G-Protein Coupled Receptors.G-protein-coupled receptors (GPCRs) constitute a vast protein family that encompasses a wide range of functions (including various autocrine, paracrine and endocrine processes). They show considerable diversity at the sequence level, on the basis of which they can be separated into distinct groups. The currently known clan members include the rhodopsin-like GPCRs, the secretin-like GPCRs, the cAMP receptors, the fungal mating pheromone receptors, and the metabotropic glutamate receptor family.

The rhodopsin-like GPCRs themselves represent a widespread protein family that includes hormone, neurotransmitter and light receptors, all of which transduce extracellular signals through interaction with guanine nucleotide-binding (G) proteins. Although their activating ligands vary widely in structure and character, the amino acid sequences of the receptors are very similar and are believed to adopt a common structural framework comprising 7 transmembrane (TM) helices The human mas oncogene was originally detected by its ability to transform NIH 3T3 cells. We previously showed that the protein encoded by this gene is unique among cellular oncogene products in that it has seven hydrophobic potential transmembrane domains and shares strong sequence similarity with a family of hormone-receptor proteins. We have now cloned the rat homolog of the mas oncogene, determined its DNA sequence, and examined its expression in various rat tissues. A comparison of the predicted sequences of the rat and human mas proteins shows that they are highly conserved, except in their hydrophilic amino-terminal domains. Our examination of the expression of mas, determined by RNA-protection studies, indicates that high levels of mas RNA transcripts are present in the hippocampus and cerebral cortex of the brain, but not in other neural regions or in other tissues. This pattern of expression and the similarity of mas protein to known receptor proteins suggest that mas encodes a receptor that is involved in the normal neurophysiology and/or development of specific neural tissues.

The disclosed NOV46 nucleic acid of the invention encoding a Mas Proto-Oncogene-like protein includes the nucleic acid whose sequence is provided in Table 46A or a fragment thereof. The invention also includes a mutant or variant nucleic acid any of whose bases may be changed from the corresponding base shown in Table 46A while still encoding a protein that maintains its Mas Proto-Oncogene-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 40 percent of the bases may be so changed.

The disclosed NOV46 protein of the invention includes the Mas Proto-Oncogene-like protein whose sequence is provided in Table 46B. The invention also includes a mutant or variant protein any of whose residues may be changed from the corresponding residue shown in Table 46B while still encoding a protein that maintains its Mas Proto-Oncogene-like activities and physiological functions, or a functional fragment thereof. In the mutant or variant protein, up to about 21 percent of the residues may be so changed.

The invention further encompasses antibodies and antibody fragments, such as F_{ab} or (F_{ab})₂, that bind immunospecifically to any of the proteins of the invention.

The above disclosed information suggests that this Mas Proto-Oncogene-like protein (NOV46) is a member of a “Mas Proto-Oncogene family”. Therefore, the NOV46 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV46 nucleic acids and proteins of the invention are useful in potential therapeutic applications implicated in Von Hippel-Lindau (VHL) syndrome, Alzheimer's disease, Stroke, Tuberous sclerosis, hypercalceimia, Parkinson's disease, Huntington's disease, Cerebral palsy, Epilepsy,Lesch-Nyhan syndrome, Multiple sclerosis, Ataxia-telangiectasia, Leukodystrophies, Behavioral disorders, Addiction, Anxiety, Pain, Neuroprotection, and/or other diseases and pathologies.

NOV46 nucleic acids and polypeptides are further useful in the generation of antibodies that bind immuno-specifically to the novel NOV46 substances for use in therapeutic or diagnostic methods. These antibodies may be generated according to methods known in the art, using prediction from hydrophobicity charts, as described in the “Anti-NOVX Antibodies” section below. The disclosed NOV46 protein has multiple hydrophilic regions, each of which can be used as an immunogen. These novel proteins can be used in assay systems for functional analysis of various human disorders, which will help in understanding of pathology of the disease and development of new drug targets for various disorders.

NOV47

A disclosed NOV47 nucleic acid of 523 nucleotides (also referred to as AF152363) encoding a Peptidyl-Prolyl Cis-Trans Isomerase-like protein is shown in Table 47A. An open reading frame was identified beginning with a ATG initiation codon at nucleotides 17-19 and ending with a TAA codon at nucleotides 509-511. The start and stop codons are shown in bold in Table 47A, and the 5′ and 3′ untranslated regions, if any, are underlined.

TABLE 47A
NOV47 nucleotide sequence.

(SEQ ID NO:179)

CCCCGTATTACCAGCTATGGTCAACCCCACTGTTTTCTTCGACATTGCTGTCAATAGCGAGCCCTTGGGCTG
CGTCTCCTTCGAGCTGTTTGCAGACAAGCTTCCAAAGACAGCAGAAAATTTTCATGCTCTGAGCACTGGAGA
AAAAGGATTTGATTATGAGGGTTACTGCTTTCACAGAATTATTCCAGGGTTTGTATGTCAGGGTGGTGACTT
CACATGCCATAATGGCACTGGTAGCAAGTCCATCTACAGGGAGAAATTTGATGACGAGAACTTCATCCTGAA
GCATACAGGTCCTGGCATCCTGTCCATGGCAAATGCTGGACCCAACGCAAATGGTTCCCAGTTTTTCATGTG
CCCTGCCAAGACCAAGTGGTTGGATGGCAAGCAAGTGGTCTTTGGCAGGGTGAAAGAAGGCATGGATATTGT
GGAGGCCATGGAGCGCTTTGTGTTCAGGAATGGCAAGACTAGCAAGAAGGTCACTATTGCTGACTGTGGACA
GCTCTAATAAGTTTGACTT

In a search of public sequence databases, the NOV47 nucleic acid sequence, located on chromosome 3, has 523 of 523 bases (100%) identical to a gb:GENBANK-ID:AF152363|acc:AF152363.1 mRNA from Homo sapiens (constitutive fragile region FRA3B sequence) (E=5.7e⁻¹¹¹).

The disclosed NOV47 polypeptide (SEQ ID NO:180) encoded by SEQ ID NO:179 has 164 amino acid residues and is presented in Table 47B using the one-letter amino acid code. Signal P, Psort and/or Hydropathy results predict that NOV47 has no signal peptide and is likely to be localized to the microbody (peroxisome) with a certainty of 0.6400. Alternatively, NOV47 may also localize to the plasma membrane with a certainty of 0.6000, to the cytoplasm with a certainty of 0.4500, or to the mitochondrial matrix space with a certainty of 0.1000.

TABLE 47B
Encoded NOV47 protein sequence.

(SEQ ID NO:180)

MVNPTVFFDIAVNSEPLGCVSFELFADKLPKTAENFHALSTGEKGFDYEGYCFHRIIPGFVCQGGDFTCHNG
TGSKSIYREKFDDENFILKHTGPGILSMANAGPNANGSQFFMCPAKTKWLDGKQVVFGRVKEGMDIVEAMER
FVFRNGKTSKKVTIADCGQL

A search of sequence databases reveals that the NOV47 amino acid sequence has 141 of 164 amino acid residues (85%) identical to, and 151 of 164 amino acid residues (92%) similar to, the 165 amino acid residue ptnr:pir-id:CSHUA protein from human (peptidylprolyl isomerase (EC 5.2.1.8) A) (E=5.6e⁻⁷⁵).

NOV47 is predicted to be expressed in small intestine because of the expression pattern of (GENBANK-ID: gb:GENBANK-ID:E02765|acc: E02765.1) a closely related Sus scrofa peptidyl-prolyl cis-trans isomerase A sequence homolog.

NOV47 also has homology to the amino acid sequences shown in the BLASTP data listed in Table 47C.

TABLE 47C
BLAST results for NOV47

| Gene Index/ | | Length | Identity | Positives | |
|---|---|---|---|---|---|
| Identifier | Protein/Organism | (aa) | (%) | (%) | Expect |
| gi\|17440554\|ref\|XP_{—} | similar to | 164 | 164/164 | 164/164 | 3e−91 |
| 067503.1\| | PEPTIDYL-PROLYL | | (100%) | (100%) | |
| (XM_067503) | CIS-TRANS | | | | |
| | ISOMERASE | | | | |
| | A(PPIASE) | | | | |
| | (ROTAMASE) | | | | |
| | (CYCLOPHILIN A) | | | | |
| | (CYCLOSPORIN A- | | | | |
| | BINDING PROTEIN) | | | | |
| | (H. sapiens) | | | | |
| | [Homo sapiens] | | | | |
| gi\|12804335\|gb\|AAH03026.1\| | Unknown (protein | 174 | 141/164 | 151/164 | 3e−76 |
| AAH03026 | for | | (85%) | (91%) | |
| (BC003026) | IMAGE: 2823490) | | | | |
| | [Homo sapiens] | | | | |
| gi\|4033689\|sp\|P04374\| | PEPTIDYL-PROLYL | 164 | 141/164 | 151/164 | 1e−75 |
| CYPH_BOVIN | CIS-TRANS | | (85%) | (91%) | |
| | ISOMERASE A | | | | |
| | (PPIASE) | | | | |
| | (ROTAMASE) | | | | |
| | (CYCLOPHILIN A) | | | | |
| | (CYCLOSPORIN A- | | | | |
| | BINDING PROTEIN) | | | | |
| gi\|10863927\|ref\|NP_{—} | peptidylprolyl | 165 | 141/164 | 151/164 | 2e−75 |
| 066953.1\| | isomerase A | | (85%) | (91%) | |
| (NM_021130) | (cyclophilin A) | | | | |
| | [Homo sapiens] | | | | |
| gi\|68401\|pir\|\|CSBOAB | peptidylprolyl | 163 | 140/163 | 150/163 | 5e−75 |
| | isomerase (EC | | (85%) | (91%) | |
| | 5.2.1.8) A - | | | | |
| | bovine | | | | |

The homology between these and other sequences is shown graphically in the ClustalW analysis shown in Table 47D. In the ClustalW alignment of the NOV47 protein, as well as all other ClustalW analyses herein, the black outlined amino acid residues indicate regions of conserved sequence (i.e., regions that may be required to preserve structural or functional properties), whereas non-highlighted amino acid residues are less conserved and can potentially be altered to a much broader extent without altering protein structure or function.

[image]

Table 47E lists the domain descriptions from DOMAIN analysis results against NOV47. This indicates that the NOV47 sequence has properties similar to those of other proteins known to contain this domain.

TABLE 47E
Domain Analysis of NOV47

gnl|Pfam|pfam00160, pro_isomerase, Cyclophilin type peptidyl-prolyl
cis-trans isomerase (SEQ ID NO:839)
CD-Length = 162 residues, 100.0% aligned
Score = 219 bits (558), Expect = 1e−58

| | | | |
|---|---|---|---|
| NOV47: | 5 | TVFFDIAVNSEPLGCVSFELFADKLPKTAENFHALSTGEKGFD-YEGYCFHRIIPGFVCQ | 63 |
| | | \|+\|\|\| + +\|\|\| + \|\|\|\| \| +\|\|\| \|\|\| \|\| \|\|\|\|\|\| \|+\| \|\|\|+\|\| \|+ \| | |
| Sbjct: | 1 | KVYFDITIGGKPLGRIVFELFGDVVPKTVENFRALCTGEKGFGFYKGSTFHRVIPNFMIQ | 60 |
| NOV47: | 64 | GGDFTCHNGTGSKSIYREKFDDENFILKHTGPGILSMANAGPNANGSQFFMCPAKTKWLD | 123 |
| | | \|\|\|\|\| \|\|\|\| \|\|\|\| \|\|\| \|\|\|\| \|\|\| \|\| \|\|\|\|\|\|\|\|\| \|\|\|\|\|\|+ \| \|\|\| | |
| Sbjct: | 61 | GGDFTRGNGTGGKSIYGEKFKDENFNLKHDRPGTLSMANAGPNTNGSQFFITTVATPWLD | 120 |
| NOV47: | 124 | GKQVVFGRVKEGMDIVEAMERFVFRNG=KTSKKVTIADCGQL 164 | |
| | | \|\| \|\|\|\|+\| \|\|\|\|+\|+ +\| +\| \|\| \| \|\|\|\|\|\|\| | |
| Sbjct: | 121 | GKHVVFGKVVEGMDVVDKIENVGTDSGDVPSKDVKIADCGQL 162 | |

Cyclophilin A (peptidyl-prolyl cis-trans isomerase A) is the major high-affinity binding protein in vertebrates for the immunosuppressive drug cyclosporin A (CSA). Because of its dramatic effects on decreasing morbidity and increasing survival rates in human transplants, the molecular mechanism of immunosuppression by cyclosporin A has been a matter of much interest. Cyclophilin A is a member of the immunophilin class of proteins that all possess peptidyl-prolyl cis-trans isomerase (PPIase) activity and, therefore, are believed to be involved in protein folding and/or intracellular protein transport. PPIase accelerates protein folding by catalyzing the cis-trans isomerization of proline imidic peptide bonds in oligopeptides. It is probable that CSA mediates some of its effects via an inhibitory action on PPIase. Cyclophilin is a cytosolic protein that belongs to a family of isozymes, including cyclophilins B and C, PPIase, and natural killer cell cyclophilin-related protein. The sequences of the different forms of cyclophilin-type PPIases are well conserved. Additional interest in cyclophilin A stems from studies performed by Luban et al. (1993), who showed that cyclophilin A binds to the gag protein of human immunodeficiency virus type 1 (HIV-1). This interaction can be inhibited by the immunosuppressant cyclosporin A and also by nonimmunosuppressive, cyclophilin A-binding cyclosporin A derivatives, which were also shown to exhibit potent anti-HIV-1 activity. Thus, cyclophilin A may have an essential function in HIV-1 replication.

The disclosed NOV47 nucleic acid of the invention encoding a Peptidyl-Prolyl Cis-Trans Isomerase-like protein includes the nucleic acid whose sequence is provided in Table 47A or a fragment thereof. The invention also includes a mutant or variant nucleic acid any of whose bases may be changed from the corresponding base shown in Table 47A while still encoding a protein that maintains its Peptidyl-Prolyl Cis-Trans Isomerase-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 1 percent of the bases may be so changed.

The disclosed NOV47 protein of the invention includes the Peptidyl-Prolyl Cis-Trans Isomerase-like protein whose sequence is provided in Table 47B. The invention also includes a mutant or variant protein any of whose residues may be changed from the corresponding residue shown in Table 47B while still encoding a protein thatmaintains its Peptidyl-Prolyl Cis-Trans Isomerase-like activities and physiological functions, or a functional fragment thereof. In the mutant or variant protein, up to about 15 percent of the residues may be so changed.

The invention further encompasses antibodies and antibody fragments, such as F_{ab} or (F_{ab})₂, that bind immunospecifically to any of the proteins of the invention.

The above disclosed information suggests that this Peptidyl-Prolyl Cis-Trans Isomerase-like protein (NOV47) is a member of a “Peptidyl-Prolyl Cis-Trans Isomerase family”. Therefore, the NOV47 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV47 nucleic acids and proteins of the invention are useful in potential therapeutic applications implicated in inflammatory bowel disease, diverticular disease, and/or other diseases and pathologies.

NOV47 nucleic acids and polypeptides are further useful in the generation of antibodies that bind immuno-specifically to the novel NOV47 substances for use in therapeutic or diagnostic methods. These antibodies may be generated according to methods known in the art, using prediction from hydrophobicity charts, as described in the “Anti-NOVX Antibodies” section below. The disclosed NOV47 protein has multiple hydrophilic regions, each of which can be used as an immunogen. These novel proteins can be used in assay systems for functional analysis of various human disorders, which will help in understanding of pathology of the disease and development of new drug targets for various disorders.

NOV48

NOV48 includes three novel Phospholipase C Delta-4-like proteins disclosed below. The disclosed sequences have been named NOV48a and NOV48b.

NOV48a

A disclosed NOV48a nucleic acid of 3238 nucleotides (also referred to as CG56743-01) encoding a Phospholipase C Delta-4-like protein is shown in Table 48A. An open reading frame was identified beginning with a ATG initiation codon at nucleotides 370-372 and ending with a TGA codon at nucleotides 2626-2628. The start and stop codons are shown in bold in Table 48A, and the 5′ and 3′ untranslated regions, if any, are underlined.

TABLE 48A
NOV48a nucleotide sequence.

(SEQ ID NO:181)

TTCTGATCTTTATGTGATTTGAGGCAGGTTTCTAAACCTATCTAAAGTGTCAGAGTCACTAAACTCAAAATT
AGAAGCAAAAATCAGCTACAGACTATCTTCAAGATTCACCCAGAGCCCTTTGCTCTTCCTTGCTCCTTTAGG
TGATCTGGTGCCAGCTGGTGGAACAGTGGGTGATGGCGTCCCTGCTGCAAGACCGTGAGTGCCGGGGCCCCT
GCAGGGGAAGAGGCCTTAGTGTACAGCTCAGGGAAGGGAAGGAGGTTGGACCCCTGTTCCAGAGCTCTCCCT
GGGCCTGCTACCCTCTCTGCTGGCTACCTAACCCCTGCTTTTCCTGACCTAGAGCTGACCACTCATCAGGAC
TTGCTGCTGATGCAGGAAGGCATGCCGATGCGCAAGGTGAGGTCCAAAAGCTGGAAGAAGCTAAGATACTTC
AGACTTCAGAATGACGGCATGACAGTCTGGCATGCACGGCAGGCCAGGGGCAGTGCCAAGCCCAGCGTCTCA
ATCTCTGATGTGGAGACAATACGTAATGGCCATGATTCCGAGTTGCTGCGTAGCCTGGCAGAGGAGCTCCCC
CTGGAGCAGGGCTTCACCATTGTCTTCCATGGCCGCCGCTCCAACCTGGACCTGATGGCCAACAGTGTTGAG
GAGGCCCAGATATGGATGCGAGGGCTCCAGCTGTTGGTGGATCTTGTCACCAGCATGGACCATCAGGAGCGC
CTGGACCAGTATCTGAGCGATTGGTTTCAACGTGGAGACAAAAATCAGGATGGTAAGATGAGTTTCCAAGAA
GTTCAGCGGTTATTGCACCTAATGAATGTGGAAATGGACCAAGAATATGCCTTCAGTCTCTTGCAGGCAGCA
GACACGTCCCAGTCTGGAACCCTGGAAGGAGAAGAATTCGTACAGTTCTATAAGGCATTGACTAAACGTGCT
GAGGTGCAGGAACTGTTTGAAAGTTTTTCAGCTGATGGGCAGAAGCTGACTCTGCTGGAATTTTTGGATTTC
CTCCAAGAGGAGCAGAAGGAGAGAGACTGCACCTCTGAGCTTGCTCTGGAACTCATTGACCGCTATGAACCT
TCAGACAGTGGTAAGCTGCGGCATGTGCTGAGTATGGATGGCTTCCTCAGCTACCTCTGCTCTAAGGATGGA
GACATCTTCAACCCAGCCTGCCTCCCCATCTATCAGGATATGACTCAACCCCTGAACCACTACTTCATCTGC
TCTTCTCATAACACCTACCTAGTGGGGGACCAGCTTTGTGGCCAGAGCAGCGTCGAGGGATATATACGGGCC
CTGAAGCGGGGGTGCCGCTGCGTGGAGGTGGATGTATGGGATGGACCTAGCGGGGAACCTGTCGTTTACCAC
GGACACACCCTGACCTCCCGCATCCTGTTCAAAGATGTCGTGGCCACAGTAGCACAGTATGCCTTCCAGACA
TCAGACTACCCAGTCATCTTGTCCCTGGAGACCCACTGCAGCTGGGAGCAGCAGCAGACCATGGCCCGTCAT
CTGACTGAGATCCTGGGGGAGCAGCTGCTGAGCACCACCTTGGATGGGGTGCTGCCCACTCAGCTGCCCTCG
CCTGAGGAGCTTCGGAGGAAGATCCTGGTGAAGGGGAAGAAGTTAACACTTGAGGAAGACCTGGAATATGAG
GAAGAGGAAGCAGAACCTGAGTTGGAAGAGTCAGAATTGGCGCTGGAGTCCCAGTTTGAGACTGAGCCTGAG
CCCCAGGAGCAGAACCTTCAGAATAAGGACAAAAAGAAGGTAAGCCAGCTTCTCCAGAAATCCAAGCCCATC
TTGTGTCCAGCCCTCTCTTCCCTGGTTATCTACTTGAAGTCTGTCTCATTCCGCAGCTTCACACATTCAAAG
GAGCACTACCACTTCTACGAGATATCATCTTTCTCTGAAACCAAGGCCAAGCGCCTCATCAAGGAGGCTGGC
AATGAGTTTGTGCAGCACAATACTTGGCAGTTAAGCCGTGTGTATCCCAGCGGCCTGAGGACAGACTCTTCC
AACTACTACAACCCCCAGGAACTCTGGAATGCAGGCTGCCAGATGGTGGCCATGAATATGCAGACTGCAGGG
CTTGAAATGGACATCTGTGATGGGCATTTCCGCCAGAATGGCGGCTGTGGCTATGTGCTGAAGCCAGACTTC
CTGCGTGATATCCAGAGTTCTTTCCACCCTGAGAAGCCCATCAGCCCTTTCAAAGCCCAGACTCTCTTAAAC
CAGGTGATCAGCGGTCAGCAACTCCCCAAAGTGGACAAGACCAAAGAGGGGTCCATTGTGGATCCACTGGTG
AAAGTGCAGATCTTTGGCGTTCGTCTAGACACAGCACGGCAGGAGACCAACTATGTGGAGAACAATGGTTTT
AATCCATACTGGGGGCAGACACTATGTTTCCGGGTGCTGGTGCCTGAACTTGCCATGCTGCGTTTTGTGGTA
ATGGATTATGACTGGAAATCCCGAAATGACTTTATTGGTCAGTACACCCTGCCTTGGACCTGCATGCAACAA
GGTTACCGCCACATTCACCTGCTGTCCAAAGATGGCATCAGCCTCCGCCCAGCTTCCATCTTTGTGTATATC
TGCATCCAGGAAGGCCTGGAGGGGGATGAGTCCTGAGGTGGGCATTTCACGGGAAGGGTTGGTGTGCTGGCT
TTAGACGGGGAGAAACATCTGGAAGGATGCTCGAGAGAACAAATGGAGGTGGTGAAAATCAAGCTTTGGATT
GTGCATTCCTAGGCACAAAATTACCTCATTCTTCCTAACAAGCAATCTGGGACCTGATTTTCCACCTTTTTT
CTCTTTTCTTCCCTTCCTTTGTTTTCATAAGCCTTTGGTATCTTTCCTGCCCTTTTCCTTTGTGTACTCTAT
ACTGGAGTTCCCTTCTTCCTCTTGCTGTAGGCTCAATCCCATACCGACATCTACAACTAATCTTTCCCATCA
ACTCTGTGTGAAGGCAGGTTGCAACTAGAAATTCAGAGGGGCTTGGAATAGAGAAACCTAAAGAAGCATCAT
CCCCTCCATCCCCAACTTCCTCAAAGCCCAAAGCCAAGGGAAGGATAAATCAAGGCTCAAGGCTTCCCCAGC
AAAGATTAGGGAAAGAGACTTGACCCCAGGACTGTACTACGACTCTTAAGAGAACACTGCACAGCACTCAAA
GTCCCCCACTGGACTGCTTCCTCCTTAGCCCCACTGGTATAAATACATCTCTCTCCAATTTGGCTTCAAA

In a search of public sequence databases, the NOV48a nucleic acid sequence, located on chromosome 3, has 1279 of 1285 bases (99%) identical to a gb:GENBANK-ID:AK023083|acc:AK023083.1 mRNA from Homo sapiens (cDNA FLJ13021 fis, clone NT2RP3000742, weakly similar to 1-Phosphatidylinositol-4,5-Bisphosphate Phosphodiesterase Delta 1 (EC 3.1.4.11)) (E=0.0).

The disclosed NOV48a polypeptide (SEQ ID NO:182) encoded by SEQ ID NO:181 has 752 amino acid residues and is presented in Table 48B using the one-letter amino acid code. Signal P, Psort and/or Hydropathy results predict that NOV48a has no signal peptide and is likely to be localized to the cytoplasm with a certainty of 0.4500. Alternatively, NOV48a may also localize to the microbody (peroxisome) with a certainty of 0.1265, to the mitochondrial matrix space with a certainty of 0.1000, or to the lysosome (lumen) with a certainty of 0.1000.

TABLE 48B
Encoded NOV48a protein sequence.

(SEQ ID NO:182)

MQEGMPMRKVRSKSWKKLRYFRLQNDGMTVWHARQARGSAKPSVSISDVETIRNGHDSELLRSLAEELPLEQ
GFTIVFHGRRSNLDLMANSVEEAQIWMRGLQLLVDLVTSMDHQERLDQYLSDWFQRGDKNQDGKMSFQEVQR
LLHLMNVEMDQEYAFSLLQAADTSQSGTLEGEEFVQFYKALTKRAEVQELFESFSADGQKLTLLEFLDFLQE
EQKERDCTSELALELIDRYEPSDSGKLRHVLSMDGFLSYLCSKDGDIFNPACLPIYQDMTQPLNHYFICSSH
NTYLVGDQLCGQSSVEGYIRALKRGCRCVEVDVWDGPSGEPVVYHGHTLTSRILFKDVVATVAQYAFQTSDY
PVILSLETHCSWEQQQTMARHLTEILGEQLLSTTLDGVLPTQLPSPEELRRKILVKGKKLTLEEDLEYEEEE
AEPELEESELALESQFETEPEPQEQNLQNKDKKKVSQLLQKSKPILCPALSSLVIYLKSVSFRSFTHSKEHY
HFYEISSFSETKAKRLIKEAGNEFVQHNTWQLSRVYPSGLRTDSSNYYNPQELWNAGCQMVAMNMQTAGLEM
DICDGHFRQNGGCGYVLKPDFLRDIQSSFHPEKPISPFKAQTLLNQVISGQQLPKVDKTKEGSIVDPLVKVQ
IFGVRLDTARQETNYVENNGFNPYWGQTLCFRVLVPELAMLRFVVMDYDWKSRNDFIGQYTLPWTCMQQGYR
HIHLLSKDGISLRPASIFVYICIQEGLEGDES

A search of sequence databases reveals that the NOV48a amino acid sequence has 619 of 752 amino acid residues (82%) identical to, and 675 of 752 amino acid residues (89%) similar to, the 764 amino acid residue ptnr:pir-id:S14113 protein from bovine (1-phosphatidylinositol-4,5-bisphosphate phosphodiesterase (EC 3.1.4.11) delta-2) (E=0.0).

NOV48a is predicted to be expressed in at least Amygdala, Bone Marrow, Brain, Epidermis, Heart, Hypothalamus, Lung, Mammary gland/Breast, Pituitary Gland, Placenta, Retina, Skeletal Muscle, Small Intestine, Stomach. This information was derived by determining the tissue sources of the sequences that were included in the invention including but not limited to SeqCalling sources, Public EST sources, Literature sources, and/or RACE sources.

NOV48b

In the present invention, the target sequence identified previously, NOV48a, was subjected to the exon linking process to confirm the sequence. PCR primers were designed by starting at the most upstream sequence available, for the forward primer, and at the most downstream sequence available for the reverse primer. In each case, the sequence was examined, walking inward from the respective termini toward the coding sequence, until a suitable sequence that is either unique or highly selective was encountered, or, in the case of the reverse primer, until the stop codon was reached. Such primers were designed based on in silico predictions for the full length cDNA, part (one or more exons) of the DNA or protein sequence of the target sequence, or by translated homology of the predicted exons to closely related human sequences sequences from other species. These primers were then employed in PCR amplification based ont the following pool of human cDNAs: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea, uterus. Usually the resulting amplicons were gel purified, cloned and sequenced to high redundancy. The resulting sequences from all clones were assembled with themselves, with other fragments in CuraGen Corporation's database and with public ESTs. Fragments and ESTs were included as components for an assembly when the extent of their identity with another component of the assembly was at least 95% over 50 bp. In addition, sequence traces were evaluated manually and edited for corrections if appropriate. These procedures provide the sequence reported below, which is designated NOV48b. This differs from the previously identified sequence (NOV48a) in having a deletion of 6 amino acids in one region and one amino acid at another region.

A disclosed NOV48b nucleic acid of 2341 nucleotides (also referred to as CG56743-02) encoding a Phospholipase C Delta-4-like protein is shown in Table 48C. An open reading frame was identified beginning with a ATG initiation codon at nucleotides 55-57 and ending with a TGA codon at nucleotides 2278-2280. The start and stop codons are shown in bold in Table 48C, and the 5′ and 3′ untranslated regions, if any, are underlined.

TABLE 48C
NOV48b nucleotide sequence.

(SEQ ID NO:183)

TTTCCTGACCTAGAGCTGACCACTGATCAGGGCTTGCTGCTGGTGCAGGAAGGCATGCCGATGCGCAAGGTG
AGGTCCAAAAGCTGGAAGAAGCTAAGATACTTCAGATTTCAGAATGACGGCATGACAGTCTGGCATGCACGG
CAGGCCAGGGGCAGTGCCAAGCCCAGCTTCTCAATCTCTGATGTGGAGACAATACGTAATGGCCATGATTCC
GAGTTGCTGCGTAGCCTGGCAGAGGAGCTCCCCCTGGAGCAGGGCTTCACCATTGTCTTCCATGGCCGCCGC
TCCAACCTGGACCTGATGGCCAACAGTGTTGAGGGGGCCCAGATATGGATGCGAGGGCTCCAGCTGTTGGTG
GATCTTGTCACCAGCATGGACCATCAGGAGCGCCTGGACCAATGGCTGAGCGATTGGTTTCAACGTGGAGAC
AAAAATCAGGATGGTAAGATGAGTTTCCAAGAAGTTCAGCGGTTATTGCACCTAATGAATGTGGAAATGGAC
CAAGAATATGCCTTCAGTCTTTTTCAGGCAGCAGACACGTCCCAGTCTGGAACCCTGGAAGGAGAAGAATTC
GTACAGTTCTATAAGGCATTGACTAAACGTGCTGAGGTGCAGGAACTGTTTGAAAGTTTTTCAGCTGATGGG
CAGAAGCTGACTCTGCTGGAATTTTTGGATTTCCTCCAAGAGGAGCAGAAGGAGAGAGACTGCACCTCTGAG
CTTGCTCTGGAACTCATTGACCGCTATGAACCTTCAGACAGTGGCAAACTGCGGCATGTGCTGAGTATGGAT
GGCTTCCTCAGCTACCTCTGCTCTAAGGATGGAGACATCTTCAACCCAGCCTGCCTCCCCATCTATCAGGAT
ATGACTCAACCCCTGAACCACTACTTCATCTGCTCTTCTCATAACACCTACCTAGTGGGGGACCAGCTTTGT
GGCCAGAGCAGCGTCGAGGGATATATACGGGCCCTGAAGCGGGGGTGCCGCTGCGTGGAGGTGGATGTATGG
GATGGACCTAGCGGGGAACCTGTCGTTTACCACGGACACACCCCGACCTCCCGCATCCTGTTCAAAGATGTC
GTGGCCACAGTAGCACAGTATGCCTTCCAGACATCAGACTACCCAGTCATCTTGTCCCTGGAGACCCACTGC
AGCTGGGAGCAGCAGCAGACCATGGCCCGTCATCTGACTGAGATCCTGGGGGAGCAGCTGCTGAGCACCACC
TTGGATGGGGTGCTGCCCACTCAGCTGCCCTCGCCTGAGGAGCTTCGGAGGAAGATCCTGGTGAAGGGGAAG
AAGTTAACACTTGAGGAAGACCTGGAATATGAGGAAGAGGAAGCAGAACCTGAGTTGGAAGAGTCAGAATTG
GCGCTGGAGTCCCAGTTTGAGACTGAGCCTGAGCCCCAGGAGCAGAACCTTCAGAATAAGGACAAAAAGAAG
AAATCCAAGCCCATCTTGTGTCCAGCCCTCTCTTCCCTGGTTATCTACTTGAAGTCTGTCTCATTCCGCAGC
TTCACACATTCAAAGGAGCACTACCACTTCTACGAGATATCATCTTTCTCTGAAACCAAGGCCAAGCGCCTC
ATCAAGGAGGCTGGCAATGAGTTTGTGCAGCACAATACTTGGCAGTTAAGCCGTGTGTATCCCAGCGGCCTG
AGGACAGACTCTTCCAACTACAACCCCCAGGAACTCTGGAATGCAGGCTGCCGGATGGTGGCCATGAATATG
CAGACTGCAGGGCTTGAAATGGACATCTGTGATGGGCATTTCCGCCAGAATGGCGGCTGTGGCTATGTGCTG
AAGCCAGACTTCCTGCGTGATATCCAGAGTTCTTTCCACCCTGAGAAGCCCATCAGCCCTTTCAAAGCCCAG
ACTCTCTTAATCCAGGTGATCAGCGGTCAGCAACTCCCCAAAGTGGACAAGACCAAAGAGGGGTCCATTGTG
GATCCACTGGTGAAAGTGCAGATCTTTGGCGTTCGTCTAGACACAGCACGGCAGGAGACCAACTATGTGGAG
AACAATGGTTTTAATCCATACTGGGGGCAGACACTATGTTTCCGGGTGCTGGTGCCTGAACTTGCCATGCTG
CGTTTTGTGGTAATGGATTATGACTGGAAATCCCGAAATGACTTTATTGGTCAGTACACCCTGCCTTGGACC
TGCATGCAACAAGGTTACCGCCACATTCACCTGCTGTCCAAAGATGGCATCAGCCTCCGCCCAGCTTCCATC
TTTGTGTATATCTGCATCCAGGAAGGCCTGGAGGGGGGTGAGTCCTGAGGTGGGCATTTCACGGGAAGGGTT
GGTGTGCTGGCTTTAGACGGGGAGAAACATCTGGAAG

In a search of public sequence databases, the NOV48b nucleic acid sequence, located on chromosome 2, has 1069 of 1075 bases (99%) identical to a gb:GENBANK-ID:AK023083|acc:AK023083.1 mRNA from Homo sapiens (cDNA FLJ13021 fis, clone NT2RP3000742, weakly similar to 1-Phosphatidylinositol-4,5-Bisphosphate Phosphodiesterase Delta 1 (EC 3.1.4.11)) (E=0.0).

The disclosed NOV48b polypeptide (SEQ ID NO:184) encoded by SEQ ID NO:183 has 741 amino acid residues and is presented in Table 48D using the one-letter amino acid code. Signal P, Psort and/or Hydropathy results predict that NOV48b has no signal peptide and is likely to be localized to the mitochondrial matrix space with a certainty of 0.6523. Alternatively, NOV48b may also localize to the mitochondrial inner membrane with a certainty of 0.3462, to the mitochondrial intermembrane space with a certainty of 0.3462, or to the mitochondrial outer membrane with a certainty of 0.3462.

TABLE 48D
Encoded NOV48b protein sequence.

(SEQ ID NO:184)

MPMRKVRSKSWKKLRYFRFQNDGMTVWHARQARGSAKPSFSISDVETIRNGHDSELLRSLAEELPLEQGFTI
VFHGRRSNLDLMANSVEGAQIWMRGLQLLVDLVTSMDHQERLDQWLSDWFQRGDKNQDGKMSFQEVQRLLHL
MNVEMDQEYAFSLFQAADTSQSGTLEGEEFVQFYKALTKRAEVQELFESFSADGQKLTLLEFLDFLQEEQKE
RDCTSELALELIDRYEPSDSGKLRHVLSMDGFLSYLCSKDGDIFNPACLPIYQDMTQPLNHYFICSSHNTYL
VGDQLCGQSSVEGYIRALKRGCRCVEVDVWDGPSGEPVVYHGHTPTSRILFKDVVATVAQYAFQTSDYPVIL
SLETHCSWEQQQTMARHLTEILGEQLLSTTLDGVLPTQLPSPEELRRKILVKGKKLTLEEDLEYEEEEAEPE
LEESELALESQFETEPEPQEQNLQNKDKKKKSKPILCPALSSLVIYLKSVSFRSFTHSKEHYHFYEISSFSE
TKAKRLIKEAGNEFVQHNTWQLSRVYPSGLRTDSSNYNPQELWNAGCRMVAMNMQTAGLEMDICDGHFRQNG
GCGYVLKPDFLRDIQSSFHPEKPISPFKAQTLLIQVISGQQLPKVDKTKEGSIVDPLVKVQIFGVRLDTARQ
ETNYVENNGFNPYWGQTLCFRVLVPELAMLRFVVMDYDWKSRNDFIGQYTLPWTCMQQGYRHIHLLSKDGIS
LRPASIFVYICIQEGLEGGES

A search of sequence databases reveals that the NOV48b amino acid sequence has 736 of 741 amino acid residues (99%) identical to, and 737 of 741 amino acid residues (99%) similar to, the 762 amino acid residue ptnr:TREMBLNEW-ACC:AAH06355 protein from Homo sapiens (Human) (Unknown (Protein For MGC:12837)) (E=0.0).

NOV48b is predicted to be expressed in at least Heart, Stomach, Small Intestine, Bone Marrow, Skeletal Muscle, Brain, Hypothalamus, Pituitary Gland. The sequence is predicted to have the expression pattern of (GENBANK-ID: gb:GENBANK-ID:AK023083|acc:AK023083.1) a closely related Homo sapiens cDNA FLJ13021 fis, clone NT2RP3000742, weakly similar to 1-Phosphatidylinositol-4,5-Bisphosphate Phosphodiesterase Delta 1 (EC 3.1.4.11) homolog.

NOV48a also has homology to the amino acid sequences shown in the BLASTP data listed in Table 48E.

TABLE 48E
BLAST results for NOV48a

| Gene Index/ | | Length | Identity | Positives | |
|---|---|---|---|---|---|
| Identifier | Protein/Organism | (aa) | (%) | (%) | Expect |
| gi\|14249340\|ref\|NP_{—} | hypothetical | 762 | 736/752 | 738/752 | 0.0 |
| 116115.1\| | protein MGC12837 | | (97%) | (97%) | |
| (NM_032726) | [Homo sapiens] | | | | |
| gi\|108854\|pir\|\|S14113 | 1- | 764 | 613/757 | 671/757 | 0.0 |
| | phosphatidylinositol- | | (80%) | (87%) | |
| | 4,5- | | | | |
| | bisphosphate | | | | |
| | phosphodiesterase | | | | |
| | (EC3.1.4.11) | | | | |
| | delta-2 - bovine | | | | |
| gi\|18093100\|ref\|NP_{—} | phospholipase C, | 772 | 550/755 | 631/755 | 0.0 |
| 542419.1\| | delta 4 [Rattus | | (72%) | (82%) | |
| (NM_080688) | norvegicus] | | | | |
| gi\|1304189\|dbj\|BAA09046.1\| | phodpholipase C | 771 | 548/756 | 629/756 | 0.0 |
| (D50455) | delta4 [Rattus | | (72%) | (82%) | |
| | norvegicus] | | | | |
| gi\|12855950\|dbj\|BAB30513.1 | data source: MGD, | 447 | 335/430 | 375/430 | 0.0 |
| (AK016945) | source | | (77%) | (86%) | |
| | key: MGI: 107469, | | | | |
| | evidence: | | | | |
| | ISS˜phospholipase | | | | |
| | C, delta | | | | |
| | 4˜putative [Mus | | | | |
| | musculus] | | | | |

The homology between these and other sequences is shown graphically in the ClustalW analysis shown in Table 48F. In the ClustalW alignment of the NOV48 protein, as well as all other ClustalW analyses herein, the black outlined amino acid residues indicate regions of conserved sequence (i.e., regions that may be required to preserve structural or functional properties), whereas non-highlighted amino acid residues are less conserved and can potentially be altered to a much broader extent without altering protein structure or function.

[image]

[image]

[image]

Tables 48G-N lists the domain descriptions from DOMAIN analysis results against NOV48. This indicates that the NOV48 sequence has properties similar to those of other proteins known to contain this domain.

TABLE 48G
Domain Analysis of NOV48

gnl|Smart|smart00148, PLCXc, Phospholipase C, catalytic domain (part);
domain X; Phosphoinositide-specific phospholipases C. These enzymes
contain 2 regions (X and Y) which together form a TIM barrel-like
structure containing the active site residues. Phospholipase C enzymes
(PI-PLC) act as signal transducers that generate two second
messengers, inositol-1,4,5-trisphosphate and diacylglycerol. The
bacterial enzyme appears to be a homologue of the mammalian PLCs.
(SEQ ID NO:840)
CD-Length = 145 residues, 100.0% aligned
Score = 202 bits (514), Expect = 6e−53

| | | | |
|---|---|---|---|
| NOV48: | 273 | QDMTQPLNHYFICSSHNTYLVGDQLCGQSSVEGYIRALKRGCRCVEVDVWDGPSGEPVVY | 332 |
| | | \|\|\|++\|\|+\|\|\|\| \|\|\|\|\|\|\| \| \|\| \|+\|\|\|\|\|\|\|+\|\|\| \|\|\|\|\|\|+\| \|\|\|\| \|\|\|\|+\| | |
| Sbjct: | 1 | QDMSKPLSHYFINSSHNTYLTGKQLWGESSVEGYIQALKHGCRCVELDCWDGPDGEPVIY | 60 |
| | | ° | |
| NOV48: | 333 | HGHTLTSRILFKDVVATVAQYAFQTSDYPVILSLETHCSWEQQQTMARHLTEILGEQLLS | 392 |
| | | \|\|\|\| \| \| +\|+ + ++\|\| \|\| \|\|\|\|\|\|\|\| \|\|\| +\|\| \|\|+ \|\| \|+ \| + | |
| Sbjct: | 61 | HGHTFTLPIKLSEVLEAIKKFAFVTSPYPVILSLENHCSPDQQAKMAQMFKEIFGDLLYT | 120 |
| NOV48: | 393 | TTLDGVLPTQLPSPEELRRKILVKGK | 418 |
| | | \| \|\|\|\|\|+\|+ \|\|\|+\|\|\| | |
| Sbjct: | 121 | PPTTSSL-EYLPSPEQLKGKILLKGK | 145 |

TABLE 48H
Domain Analysis of NOV48

gnl|Pfam|pfam00388, PI-PLC-X, Phosphatidylinositol-specific
phospholipase C, X domain. This associates with pfam00387 to form a
single structural unit. (SEQ ID NO:841)
CD-Length = 145 residues, 100.0% aligned
Score = 192 bits (489), Expect = 4e−50

| | | | |
|---|---|---|---|
| NOV48: | 274 | DMTQPLNHYFICSSHNTYLVGDQLCGQSSVEGYIRALKRGCRCVEVDVWDGPSGEPVVYH | 333 |
| | | \|\|+ \|\|+\|\|\|\| \|\|\|\|\|\|\| \| \|\| \|+\| \|\| \| + \| \|\|\|\|\|\|+\| \|\|\|\| \|\|++\|\| | |
| Sbjct: | 1 | DMSIPLSHYFISSSHNTYLTGKQLWGKSQVESYRQQLDHGCRCVELDCWDGPDDEPIIYH | 60 |
| NOV48: | 334 | GHTLTSRILFKDVVATVAQYAFQTSDYPVILSLETHCSWEQQQTMARHLTEILGEQLLST | 393 |
| | | \| \| \| \| \|\|\|+ + + \|+\|\| \|\|+\|\|\|\|\| \|\|+ +\|\|+ \|\|++ \|\| \|+ \|\|+ | |
| Sbjct: | 61 | GGTFTLEIKLKDVLEAIKDFLFKTSPYPIILSLENHCNSDQQRKMAKYFEEIFGDYLLTK | 120 |
| NOV48: | 394 | TLDGVLPTQLPSPEELRRKILVKGKK | 419 |
| | | \|\| \| \|+\|\|\| ++\|+ \|\|\|+\| \|\| | |
| Sbjct: | 121 | PLDS-LTTKLPSLKDLKGKILLKNKK | 145 |

TABLE 48I
Domain Analysis of NOV48

gnl|Smart|smart00149, PLCYc, Phospholipase C, catalytic domain (part);
domain Y; Phosphoinositide-specific phospholipases C. These enzymes
contain 2 regions (X and Y) which together form a TIM barrel-like
structure containing the active site residues. Phospholipase C enzymes
(PI-PLC) act as signal transducers that generate two second
messengers, inositol-1,4,5-trisphosphate and diacylglycerol. The
bacterial enzyme appears to be a homologue of the mammalian PLCs.
(SEQ ID NO:842)
CD-Length = 117 residues, 100.0% aligned
Score = 182 bits (462), Expect = 6e−47

| | | | |
|---|---|---|---|
| NOV48: | 482 | LSSLVIYLKSVSFRSFTHSKEHYHFYEISSFSETKAKRLIKEAGNEFVQHNTWQLSRVYP | 541 |
| | | \|\| \|\| \| \| \|\|\|\| ++\| \|\|\|+\|\|\|\|\|\|\|\|\|+\|+++\| +\|\|++\| \|\|\|\|\|\|\| | |
| Sbjct: | 1 | LSELVSYCAPVKFRSFELAEEKNPFYEMSSFSETKAKKLLEKAPTDFVRYNQRQLSRVYP | 60 |
| NOV48: | 542 | SGLRTDSSNYYNPQELWNAGCQMVAMNMQTAGLEMDICDGHFRQNGGCGYVLKPDFLR | 599 |
| | | \| \| \|\|\|\|\| \|\|\| \|\| \|\|\|\|\|\|+\| \|\| \| + \| \|\| \|\|\|\|\|\|\|\|\|\|\|\|\|\| | |
| Sbjct: | 61 | KGTRVDSSNY-NPQVFWNHGCQMVALNFQTPDKAMQLNQGMFRANGGCGYVLKPDFLR | 117 |

TABLE 48J
Domain Analysis of NOV48

gnl|Pfam|pfam00387, PI-PLC-Y, Phosphatidylinositol-specific
phospholipase C, Y domain. This associates with pfam00388 to form a
single structural unit. (SEQ ID NO:843)
CD-Length = 118 residues, 99.2% aligned
Score = 163 bits (412), Expect = 4e−41

| | | | |
|---|---|---|---|
| NOV48: | 482 | LSSLVIYLKSVSFRSFTHSKEHYHFYEISSFSETKAKRLIKEAGNEFVQHNTWQLSRVYP | 541 |
| | | \|\|+\|\| \|++\|+ \|\|\|\|+ \| \|\|+\|\|\|\|\| \|\|\|+\|+\|\|+ \|\|\|+\|\| \|\|\|\|\|\|\| | |
| Sbjct: | 2 | LSNLVNYIQSIKFRSFSLPTEKNTSYEMSSFSERKAKQLLKESPIEFVKHNKRQLSRVYP | 61 |
| | | ° | |
| NOV48: | 542 | SGLRTDSSNYYNPQELWNAGCQMVAMNMQTAGLEMDICDGHFRQNGGCGYVLKPDFLR | 599 |
| | | \| \| \|\|\|\| + \|\| \|\|\|\|\|\|\|\|\|+\| \|\|+ \| \| \| \| \| \|\|\| \|\|+\|\|\| \|\|\| | |
| Sbjct: | 62 | KGTRFDSSN-FMPQPFWNAGCQMVALNFQTSDLPMQINLGMFEYNGGSGYLLKPPFLR | 118 |

TABLE 48K
Domain Analysis of NOV48

gnl|Pfam|pfam00168, C2, C2 domain. (SEQ ID NO:844)
CD-Length = 88 residues, 95.5% aligned
Score = 88.2 bits (217), Expect = 2e−18

| | | | |
|---|---|---|---|
| NOV48: | 623 | VISGQQLPKVDKTKEGSIVDPLVKVQIFGVRLDTARQETNYVENNGFNPYWGQTLCFR-V | 681 |
| | | \|\|\| + \|\|\|+\| + \|\| \|\|\| + \| \|\| + +\| \|+ \|\| \| +\| \| \| | |
| Sbjct: | 5 | VISARNLPKMDMN---GLSDPYVKVDLDGDPKDTKKFKTKTVKKT-LNPVWNETFVFEKV | 60 |
| NOV48: | 682 | LVPELAMLRFVVMDYDWKSRNDFIGQYT | 709 |
| | | +\|+\|\| \|\|\| \| \| \| \|\|+\|\|\|\|\| \| | |
| Sbjct: | 61 | PLPDLASLRFAVYDEDRFSRDDFIGQVT | 88 |

TABLE 48L
Domain Analysis of NOV48

gnl|Smart|smart00239, C2, Protein kinase C conserved region 2 (CalB);
Ca2 + -binding motif present in phospholipases, protein kinases C. and
synaptotamins (among others). Some do not appear to contain Ca2 + -
binding sites. Particular C2s appear to bind phospholipids, inositol
polyphosphates, and intracellular proteins. Unusual occurrence in
perform. Synaptotagmin and PLC C2s are permuted in sequence with
respect to N- and C-terminal beta strands. SMART detects C2 domains
using one or both of two profiles. (SEQ ID NO:845)
CD-Length = 101 residues, 100.0% aligned
Score = 83.6 bits (205), Expect = 4e−17

| | | | |
|---|---|---|---|
| NOV48: | 618 | TLLNQVISGQQLPKVDKTKEGSIVDPLVKVQIFGVRLDTARQETNYVENNGFNPYWGQTL | 677 |
| | | \|\| ++\|\| \|\| \|\| \|\| \|\|\| + \| + + \| \|+\| \|\| \| +\| | |
| Sbjct: | 1 | TLTVKIISARNLPPKDKG---GKSDPYVKVSLDGDPREKKK--TKVVKNTL-NPVWNETF | 54 |
| NOV48: | 678 | CFRVLVPELAMLRFVVMDYDWKSRNDFIGQYTLPWTCMQQGYRHIHL | 724 |
| | | \| \| \|\|\|+ \| \| \| \| \|\|+\|\|\|\|+ \|+\| + + \| \|\| \| | |
| Sbjct: | 55 | EFEVPPPELSELEIEVYDKDRFSRDDFIGRVTIPLSDLLLGGRHEKL | 101 |

TABLE 48M
Domain Analysis of NOV48

gnl|Smart|smart00233, PH, Pleckstrin homology domain.; Domain commonly
found in eukaryotic signalling proteins. The domain family possesses
multiple functions including the abilities to bind inositol
phosphates, and various proteins. PH domains have been found to
possess inserted domains (such as in PLC gamma, syntrophins) and to be
inserted within other domains. Mutations in Brutons tyrosine kinase
(Btk) within its PH domain cause X-linked agammaglobulinaemia (XLA) in
patients. Point mutations cluster into the positively charged end of
the molecule around the predicted binding site for
phosphatidylinositol lipids. (SEQ ID NO:846)
CD-Length = 104 residues, 87.5% aligned
Score = 47.8 bits (112), Expect = 2e−06

| | | | |
|---|---|---|---|
| NOV48: | 9 | KVRSKSWKKLRYFRLQNDGMTVWHARQARGSAKPSVSIS-DVETIRNGHDSELLRSLAEE | 67 |
| | | \|\|\|\|\| \|\|\| \| \| + + +++ + \|+\|\| \|\| \|+\| \|\|+ | |
| Sbjct: | 12 | SGGKKSWKK-RYFVLFNGVLLYYKSKKKKSSSKPKGSIPLSGCTVREAPDSD-------S | 63 |
| NOV48: | 68 | LPLEQGFTIVFHGRRSNLDLMANSVEEAQIWMRGLQLLVD | 107 |
| | | + \| \|\| \|++ \| \| \| \| \|\| + \|+ \|+ + | |
| Sbjct: | 64 | DKKKNCFEIVTPDRKT-LLLQAESEEERKEWVEALRKAIA | 102 |

TABLE 48N
Domain Analysis of NOV48

gnl|Pfam|pfam00169, PH, PH domain. PH stands for pleckstrin homology
(SEQ ID NO:847)
CD-Length = 100 residues, 88.0% aligned
Score = 45.1 bits (105), Expect = 1e−06

| | | | |
|---|---|---|---|
| NOV48: | 9 | KVRSKSWKKLRYFRLQNDGMTVWHARQARGSAKPSVSISDVETIRNGHDSELLRSLAEEL | 68 |
| | | \|+ \| \|\|\| \|\|\| \| \|\| + + ++ \| \|+ +\| + \| | |
| Sbjct: | 12 | TVKKKRWKK-RYFFLFNDVLIYYKDKKKSYEPKGSIPLSGCS---------VEDVPDSEF | 61 |
| NOV48: | 69 | PLEQGFTIVFHGRRSNLDLMANSVEEAQIWMRGLQLLV | 106 |
| | | \| + + \| \| \| \|\| \| \|++ +\| + | |
| Sbjct: | 62 | KRPNCFQLRSRDGKETFILQAESEEERQDWIKAIQSAI | 99 |

Phosphatidylinositol-specific phospholipase C (EC 3.1.4.11), an eukaryotic intracellular enzyme, plays an important role in signal transduction processes. It catalyzes the hydrolysis of 1-phosphatidyl-D-myo-inositol-3,4,5-triphosphate into the second messenger molecules diacylglycerol and inositol-1,4,5-triphosphate. This catalytic process is tightly regulated by reversible phosphorylation and binding of regulatory proteins. In mammals, there are at least 6 different isoforms of PI-PLC, they differ in their domain structure, their regulation, and their tissue distribution. Lower eukaryotes also possess multiple isoforms of PI-PLC. All eukaryotic PI-PLCs contain two regions of homology, sometimes referred to as ‘X-box’ and ‘Y-box’. The order of these two regions is always the same (NH2-X—Y—COOH), but the spacing is variable. In most isoforms, the distance between these two regions is only 50-100 residues but in the gamma isoforms one PH domain, two SH2 domains, and one SH3 domain are inserted between the two PLC-specific domains. The two conserved regions have been shown to be important for the catalytic activity. At the C-terminal of the Y-box, there is a C2 domain possibly involved in Ca-dependent membrane attachment. Phosphoinositide-specific phospholipase C (PLC) mediates the cellular actions of a variety of hormones, neurotransmitters and growth factors. Agonist-dependent activation of PLC causes hydrolysis of membrane phosphatidylinositol 4,5-bisphosphate (PIP2), generating the second messengers inositol 1,4,5-trisphosphate (IP3) and diacylglycerol (DAG). IP3 binds specific intracellular receptors to trigger Ca2+ mobilisation, while DAG mediates activation of a family of protein kinase C isozymes. Based on molecular size, immunoreactivity and amino acid sequence, several subtypes have been classified. In PLC-beta subtypes, X and Y domains are separated by a stretch of 70-120 amino acids rich in Ser, Thr and acidic residues. Their C-terminus is rich in basic residues. In PLC-gammas, there is an insert of more than 400 residues containing an SH3 and two SH2 domains. PLCs show little similarity in the 300-residue N-terminal region preceding the X-domain.

The disclosed NOV48 nucleic acid of the invention encoding a Phospholipase C Delta-4-like protein includes the nucleic acid whose sequence is provided in Table 48A, 48C or a fragment thereof. The invention also includes a mutant or variant nucleic acid any of whose bases may be changed from the corresponding base shown in Table 48A or 48C while still encoding a protein that maintains its Phospholipase C Delta-4-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 1 percent of the bases may be so changed.

The disclosed NOV48 protein of the invention includes the Phospholipase C Delta-4-like protein whose sequence is provided in Table 48B or 48D. The invention also includes a mutant or variant protein any of whose residues may be changed from the corresponding residue shown in Table 48B or 48D while still encoding a protein that maintains its Phospholipase C Delta-4-like activities and physiological functions, or a functional fragment thereof. In the mutant or variant protein, up to about 28 percent of the residues may be so changed.

The invention further encompasses antibodies and antibody fragments, such as F_{ab} or (F_{ab})₂, that bind immunospecifically to any of the proteins of the invention.

The above disclosed information suggests that this Phospholipase C Delta-4-like protein (NOV48) is a member of a “Phospholipase C Delta-4 family”. Therefore, the NOV48 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV48 nucleic acids and proteins of the invention are useful in potential therapeutic applications implicated in Cardiomyopathy, Atherosclerosis, Hypertension, Congenital heart defects, Aortic stenosis Atrial septal defect (ASD), Atrioventricular (A-V) canal defect, Ductus arteriosus, Pulmonary stenosis, Subaortic stenosis, Ventricular septal defect (VSD), valve diseases, Tuberous sclerosis, Scleroderma, Obesity, Transplantation, Osteoporosis, Hypercalceimia, Arthritis, Ankylosing spondylitis, Scoliosis, Von Hippel-Lindau (VHL) syndrome, Alzheimer's disease, Stroke, Tuberous sclerosis, hypercalceimia, Parkinson's disease, Huntington's disease, Cerebral palsy, Epilepsy, Lesch-Nyhan syndrome, Multiple sclerosis, Ataxia-telangiectasia, Leukodystrophies, Behavioral disorders, Addiction, Anxiety, Pain, Neuroprotection, and/or other diseases and pathologies.

NOV48 nucleic acids and polypeptides are further useful in the generation of antibodies that bind immuno-specifically to the novel NOV48 substances for use in therapeutic or diagnostic methods. These antibodies may be generated according to methods known in the art, using prediction from hydrophobicity charts, as described in the “Anti-NOVX Antibodies” section below. The disclosed NOV48 protein has multiple hydrophilic regions, each of which can be used as an immunogen. These novel proteins can be used in assay systems for functional analysis of various human disorders, which will help in understanding of pathology of the disease and development of new drug targets for various disorders.

NOV49

A disclosed NOV49 nucleic acid of 1588 nucleotides (also referred to as CG56739-01) encoding a Leukotriene-B4 Omega-Hydroxylase-like protein is shown in Table 49A. An open reading frame was identified beginning with a ATG initiation codon at nucleotides 2-4 and ending with a TGA codon at nucleotides 1577-1579. The start and stop codons are shown in bold in Table 49A, and the 5′ and 3′ untranslated regions, if any, are underlined.

TABLE 49A
NOV49 nucleotide sequence.

(SEQ ID NO:185)

GATGTCGCTGCTGAGCCTGTCCTGGCTGGGCCTCGGGCCGGTGGCAGCATCCCCATGGCTGCTCCTGCTGCT
GGTCGGGGCCTCCTGGCTCCTGGCCCGTGTCCTGGCCTGGACGTACGCCTTCTATGACAACTGCCACCGCCT
CCAGTGTTTCCAGCAGCCTCCAAAACGGAACTGCTTTTTAGGTCACCTGAGCCTGGTGCGGGGCAATGAGGA
GGACATGAGGCTGATGGAGGATCTGGGCCACTACTTCCGTGATGTCCAACTCTGGTGGCTTGGGTCTTTCTA
CCCTGTCCTGCATCTCGTCCACCCTACGTTCACTGCCCCTGTGCTCCAGGCTTCAGCTGCTGTTGCACTCAA
GGATATGAGTTTCTATGGCTTCCTGAAGCCCTGGCTGGGTCCTGATGGGCTCCTGATTAGTGCCGGTGACAA
GTGGAGATGGCACCGCCACCTGCTCACACCTGCCTTCCACTTCAAAATCCTGAAGCCCTATGTGAAGATTTT
CAATGAGAGCACGAACATCATGCACGCCAAATGGCAACGCCTGGCCTTGGAGGGCAGTGTCCGTCTGGAAAT
GTTTGAGCACATCAGCCTCATGACCTTGGACAGTCTGCAGAAATGCATCTTCAGCTTTGACACCAATTGTCA
GGAGAAGCCCAGCGAATATATTGATGCCATCTTGGAGCTCAGTGCCCTCAGTCTGAAACGGCACCAGCACAT
CTTCCTGCTCACGGACTTCTTGTACTTCCTCACTCCCAATGGGCGACGCTTCTGCAGGGCCTGTGACATAGT
GCACAACTTCACAGATGCTGTCATCCAGGAGCGGCGTCGCACCCTCACTAGCCAGGGTGTCGATGACTTCCT
GCAGGCCAAGGCCAAGTCCAAGACTTTGGACTTCATTGACGTGCTCTTGCTGGCCAAGGATGAAAATGGAAA
GAAGTTGTCAGATGAGAACATAAGAGCGGAGGCTGACACCTTCATGTCTGGGGGCCATGACACCTCGGCCAG
TGGTCTCTCCTGGGTCCTGTACAACCTCGCGAGGTACCCAGAATACCAGGAGCACTGCCGACAGGAGGTGCA
AGAGCTCCTGAAGAACGGTGATCCTAAAGAGATTGAATGGGATGACCTGGCCCAGTTGCCCTTCCTGACCAT
GTGCCTGAAGGAGAGCCTGCGGCTGCATTCCCCAGTCTCCAGGATCCACCGCTGCTGCCCCCAGGACGGGGT
GCTCCCGGATGGCCGGGTCATCCCCAAAGGTAACACTTGCACCATCAGCATCTTTGGGATCCATCACAACCC
TTCAGTCTGGCCGGACCCGGAGGTGTATGACCCCTTTCGCTTCGACCCAGAAAATCTCCAGAAGACATCACC
TCTGGCTTTTATTCCCTTCTCAGCAGTGCCCAGGAACTGCATCGGCCAGACGTTCGCCATGGCTGAGATGAA
GGTGGTCCTGGCGCTCACGCTGCTGCGCTTCCGCGTCCTGCCGGACCACGCGGAGCCCCGCAGGAAGCTGGA
GCTGATCGTGCGCGCGGAGGATGGACTTTGGCTACGGGTGGAGCCCCTGAGCGCGGATCTGCAGTGACCCAC
CACT

In a search of public sequence databases, the NOV49 nucleic acid sequence, located on chromosome 19, has 1320 of 1584 bases (83%) identical to a gb:GENBANK-ID:HUMLB4OH|acc:D26480.1 mRNA from Homo sapiens (Human mRNA for leukotriene B4 omega-hydroxylase, complete cds) (E=9.7e⁻²³⁷).

The disclosed NOV49 polypeptide (SEQ ID NO:186) encoded by SEQ ID NO:185 has 525 amino acid residues and is presented in Table 49B using the one-letter amino acid code. Signal P, Psort and/or Hydropathy results predict that NOV49 has a signal peptide and is likely to be localized extracellularly with a certainty of 0.8200. Alternatively, NOV49 may also localize to the lysosome (lumen) with a certainty of 0.4520, to the microbody (peroxisome) with a certainty of 0.1611, or to the endoplasmic reticulum (membrane) with a certainty of 0.1000. The most likely cleavage sit for NOV49 is between positions 36 and 37: VLA-WT.

TABLE 49B
Encoded NOV49 protein sequence.

(SEQ ID NO:186)

MSLLSLSWLGLGPVAASPWLLLLLVGASWLLARVLAWTYAFYDNCHRLQCFQQPPKRNCFLGHLSLVRGNEE
DMRLMEDLGHYFRDVQLWWLGSFYPVLHLVHPTFTAPVLQASAAVALKDMSFYGFLKPWLGPDGLLISAGDK
WRWHRHLLTPAFHFKILKPYVKIFNESTNIMHAKWQRLALEGSVRLEMFEHISLMTLDSLQKCIFSFDSNCQ
EKPSEYIDAILELSALSLKRHQHIFLLTDFLYFLTPNGRRFCRACDIVHNFTDAVIQERRRTLTSQGVDDFL
QAKAKSKTLDFIDVLLLAKDENGKKLSDENIRAEADTFMSGGHDTSASGLSWVLYNLARYPEYQEHCRQEVQ
ELLKNGDPKEIEWDDLAQLPFLTMCLKESLRLHSPVSRIHRCCPQDGVLPDGRVIPKGNTCTISIFGIHHNP
SVWPDPEVYDPFRFDPENLQKTSPLAFIPFSAVPRNCIGQTFAMAEMKVVLALTLLRFRVLPDHAEPRRKLE
LIVRAEDGLWLRVEPLSADLQ

A search of sequence databases reveals that the NOV49 amino acid sequence has 397 of 521 amino acid residues (76%) identical to, and 444 of 521 amino acid residues (85%) similar to, the 520 amino acid residue ptnr:SWISSPROT-ACC:Q08477 protein from Homo sapiens (Human) (CYTOCHROME P450 4F3 (EC 1.14.13.30) (CYPIVF3) (Leukotriene-B4 Omega-Hydroxylase) (Leukotriene-B4 20-Monooxygenase) (Cytochrome P450-LTB-Omega)) (E=4.3e⁻²¹⁹).

NOV49 is predicted to be expressed in at least Prostate. This information was derived, by determining the tissue sources of the sequences that were included in the invention including but not limited to SeqCalling sources, Public EST sources, Literature sources, and/or RACE sources.

In addition, the sequence is predicted to be expressed in Bone Marrow, Peripheral Blood, Brain, Colon, Coronary Artery, Hippocampus, Kidney, Kidney Cortex, Liver, Lymph node, Pituitary Gland, and Prostate because of the expression pattern of (GENBANK-ID: gb:GENBANK-ID:HUMLB4OH|acc:D26480.1) a closely related Human mRNA for leukotriene B4 omega-hydroxylase, complete cds homolog.

NOV49 also has homology to the amino acid sequences shown in the BLASTP data listed in Table 49C.

TABLE 49C
BLAST results for NOV49

| Gene Index/ | | Length | Identity | Positives | |
|---|---|---|---|---|---|
| Identifier | Protein/Organism | (aa) | (%) | (%) | Expect |
| gi\|17456512\|ref\|XP_{—} | similar to | 491 | 452/495 | 455/495 | 0.0 |
| 065069.1\| | CYTOCHROME P450 | | (91%) | (91%) | |
| (XM_065069) | 4F6 (CYPIVF6) (H. | | | | |
| | sapiens) [Homo | | | | |
| | sapiens] | | | | |
| gi\|14767705\|ref\|XP_{—} | cytochrome P450, | 520 | 399/521 | 446/521 | 0.0 |
| 029072.1\| | subfamily IVF, | | (76%) | (85%) | |
| (XM_029072) | polypeptide 3 | | | | |
| | [Homo sapiens] | | | | |
| gi\|4503241\|ref\|NP_000887.1\| | cytochrome P450, | 520 | 397/521 | 444/521 | 0.0 |
| (NM_000896) | subfamily IVF, | | (76%) | (85%) | |
| | polypeptide | | | | |
| | 3; leukotriene B4 | | | | |
| | omega | | | | |
| | hydroxylase; | | | | |
| | leukotriene-B4 | | | | |
| | 20-monoxygenase; | | | | |
| | cytochrome P450- | | | | |
| | LTB-omega [Homo | | | | |
| | sapiens] | | | | |
| gi\|2997737\|gb\|AAC08589.1\| | cytochrome P-450 | 520 | 395/521 | 440/521 | 0.0 |
| (AF054821) | [Homo sapiens] | | (75%) | (83%) | |
| gi\|1706095\|sp\|P51871\| | CYTOCHROME P450 | 537 | 392/521 | 443/521 | 0.0 |
| CPF6_RAT | 4F6 (CYPIVF6) | | (75%) | (84%) | |

The homology between these and other sequences is shown graphically in the ClustalW analysis shown in Table 49D. In the ClustalW alignment of the NOV49 protein, as well as all other ClustalW analyses herein, the black outlined amino acid residues indicate regions of conserved sequence (i.e., regions that may be required to preserve structural or functional properties), whereas non-highlighted amino acid residues are less conserved and can potentially be altered to a much broader extent without altering protein structure or function.

[image]

[image]

[image]

Table 49F lists the domain description from DOMAIN analysis results against NOV49. This indicates that the NOV49 sequence has properties similar to those of other proteins known to contain this domain.

TABLE 49F
Domain Analysis of NOV49

gnl|Pfam|pfam00067, p450, Cytochrome P450. Cytochrome P450s are
involved in the oxidative degradation of various compounds.
Particularly well known for their role in the degradation of
environmental toxins and mutagens. Structure is mostly alpha, and
binds a heme cofactor. (SEQ ID NO:848)
CD-Length = 445 residues, 98.9% aligned
Score = 308 bits (790), Expect = 4e−85

| | | | |
|---|---|---|---|
| NOV49: | 55 | PKRNCFLGHLSLVRGNEEDMRLMEDLGHYFRDVQLWWLGSFYPVLHLVHPTFTAPVLQAS | 114 |
| | | \| +\|+\| + + + +\| + \| +\|\| \|\|+ + \| \|\| | |
| Sbjct: | 4 | PPPLPLIGNLLQLGRG--PIHSLTELRKKYGPVFTLYLGP-RPVVVVTGPEAVKEVLIDK | 60 |
| NOV49: | 115 | AAVALKDMSFYGFLKPWLGPDGLLISAGDKWRWHRHLLTPAFHFKILKPYVKIFNESTNI | 174 |
| | | \| \| \|\|\|\| \|+\| \| \| +\|\| \| \|\|\| \| \| + \| \|+ | |
| Sbjct: | 61 | GEEFAGRGDFPVF--PWLG-YGILFSNGPRWRQLRRLLTLRF-FGMGKRS-KLEERIQEE | 115 |
| NOV49: | 175 | MHAKWQRLALEGSVRLEMFEHISLMTLDSLQKCIFSFDSNCQEKPSE-YIDAILELSALS | 233 |
| | | +\|\| \| +++ \| ++ \|+ + +\| + ++ \|\| + \|\| \| | |
| Sbjct: | 116 | ARDLVERLRKEQGSPIDITELLAPAPLNVICSLLFGVRFDYEDPEFLKLIDKLNELFFLV | 175 |
| NOV49: | 234 | LKRHQHIFLLTDFLYFLTPNGRRFCRACDIVHNFTDAVIQERRRTLTSQGVDDFLQAKAK | 293 |
| | | \| \| \|\| +\| + \|+ +\| + ++ \| +\|+\|\|\| \|\| | |
| Sbjct: | 176 | SPWGQ----LLDFFRYLPGSHRKAFKAAKDLKDYLDKLIEERRETLE------------P | 219 |
| NOV49: | 294 | SKTLDFIDVLLL-AKDENGKKLSDENIRAEADTFMSGGHDTSASGLSWVLYNLARYPEYQ | 352 |
| | | \|\|+\| \|\|+ \|\| \| \| +\|+\|\| ++\| + \| \|\|++\| \|\|\| \|\| \|\|++\|\| \| | |
| Sbjct: | 220 | GDPRDFLDSLLIEAKREGGSELTDEELKATVLDLLFAGTDTTSSTLSWALYLLAKHPEVQ | 279 |
| NOV49: | 353 | EHCRQEVQELLKNGDPKEIEWDDLAQLPFLTMCLKESLRLHSPV-SRIHRCCPQDGVLPD | 411 |
| | | \|+\|+ \|++ \| + +\|\| \| +\|+\| +\|\|+\|\|\|\| \| + \| +\| + \| | |
| Sbjct: | 280 | AKLREEIDEVI--GRDRSPTYDDRANMPYLDAVIKETLRLHPVVPLLLPRVATEDTEI-D | 336 |
| NOV49: | 412 | GRVIPKGNTCTISIFGIHHNPSVWPDPEVYDPFRFDPENLQKTSPLAFIPFSAVPRNCIG | 471 |
| | | \| +\|\|\|\| ++++ +\| +\| \|+\|+\|\| +\|\| \|\| \|\| + \|\|+\|\| \| \|\|\|\|+\| | |
| Sbjct: | 337 | GYLIPKGTLVIVNLYSLHRDPKVFPNPEEFDPERFLDENGKFKKSYAFLPFGAGPRNCLG | 396 |
| NOV49: | 472 | QTFAMAEMKVVLALTLLRFRV-LPDHAEPRRKLELIVRAEDGLWLRV | 517 |
| | | + \| \|+ + \|\| \| \|\| + \| + + + ++ | |
| Sbjct: | 397 | ERLARMELFLFLATLLQRFELELVPPGDIPLTPKPLGLPSKPPLYQL | 443 |

Leukotrienes are a group of bioactive compounds that play important roles in such processes as inflammation. Kikuta et al. (1993) (J. Biol. Chem. 268: 9376-9380) isolated a cDNA for the human leukotriene B4 omega-hydroxylase (LTB4H), an enzyme which catalyzes the omega-hydroxylation of leukotriene B4. Their cDNA encoded a 520-amino acid protein with a predicted molecular weight of 59,805 Da. The deduced amino acid sequence contains a cysteine in the conserved heme-binding domain near the C-terminus, which is a characteristic feature of the cytochrome P450 superfamily; the protein shares 31 to 44% similarity with CYP4A, CYP4B, and CYP4C. Kikuta et al. (1993) (J. Biol. Chem. 268: 9376-9380) detected transcript from the LTB4H gene in polymorphonuclear leukocytes and leukocytes. Kikuta et al. (1998) (DNA Cell Biol. 17: 221-230) determined that the CYP4F3 gene contains 13 exons and spans approximately 22.2 kb. By fluorescence in situ hybridization, they mapped the CYP4F3 gene to 19p13.2. The cytochrome P450 enzymes usually act as terminal oxidases in multicomponent electron transfer chains, called P450-containing monooxygenase systems. P450-containing monooxygenase systems primarily fall into two major classes: bacterial/mitochondrial (type I), and microsomal (type II). All P450 enzymes can be categorised into two main groups, the so-called B- and E-classes: P450 proteins of prokaryotic 3-component systemsand fungal P450nor (CYP55) belong to the B-class; all other known P450 proteins from distinct systems are of the E-class. This family contains a number of subtypes of both B and E classes.

The disclosed NOV49 nucleic acid of the invention encoding a Leukotriene-B4 Omega-Hydroxylase-like protein includes the nucleic acid whose sequence is provided in Table 49A or a fragment thereof. The invention also includes a mutant or variant nucleic acid any of whose bases may be changed from the corresponding base shown in Table 49A while still encoding a protein that maintains its Leukotriene-B4 Omega-Hydroxylase-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 17 percent of the bases may be so changed.

The disclosed NOV49 protein of the invention includes the Leukotriene-B4 Omega-Hydroxylase-like protein whose sequence is provided in Table 49B. The invention also includes a mutant or variant protein any of whose residues may be changed from the corresponding residue shown in Table 49B while still encoding a protein that maintains its Leukotriene-B4 Omega-Hydroxylase-like activities and physiological functions, or a functional fragment thereof. In the mutant or variant protein, up to about 25 percent of the residues may be so changed.

The invention further encompasses antibodies and antibody fragments, such as F_{ab} or (F_{ab})₂, that bind immunospecifically to any of the proteins of the invention.

The above disclosed information suggests that this Leukotriene-B4 Omega-Hydroxylase-like protein (NOV49) is a member of a “Leukotriene-B4 Omega- Hydroxylase family”. Therefore, the NOV49 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV49 nucleic acids and proteins of the invention are useful in potential therapeutic applications implicated in Atherosclerosis, Aneurysm, Hypertension, Fibromuscular dysplasia, Stroke, Scleroderma, Obesity, Transplantation, Myocardial infarction, Embolism, Cardiovascular disorders, Bypass surgery, Osteoporosis, Hypercalceimia, Arthritis, Ankylosing spondylitis, Scoliosis, Von Hippel-Lindau (VHL) syndrome, Alzheimer's disease, Stroke, Tuberous sclerosis, hypercalceimia, Parkinson's disease, Huntington's disease, Cerebral palsy, Epilepsy, Lesch-Nyhan syndrome, Multiple sclerosis, Ataxia-telangiectasia, Leukodystrophies, Behavioral disorders, Addiction, Anxiety, Pain, Neuroprotection, Diabetes, Autoimmune disease, Renal artery stenosis, Interstitial nephritis, Glomerulonephritis, Polycystic kidney disease, Systemic lupus erythematosus, Renal tubular acidosis, IgA nephropathy, Hypercalceimia, Lesch-Nyhan syndrome, and/or other diseases and pathologies.

NOV49 nucleic acids and polypeptides are further useful in the generation of antibodies that bind immuno-specifically to the novel NOV49 substances for use in therapeutic or diagnostic methods. These antibodies may be generated according to methods known in the art, using prediction from hydrophobicity charts, as described in the “Anti-NOVX Antibodies” section below. The disclosed NOV49 protein has multiple hydrophilic regions, each of which can be used as an immunogen. These novel proteins can be used in assay systems for functional analysis of various human disorders, which will help in understanding of pathology of the disease and development of new drug targets for various disorders.

NOV50

NOV50 includes three novel Protein Arginine N-Methyltransferase 2-like proteins disclosed below. The disclosed sequences have been named NOV50a and NOV50b.

NOV50a

A disclosed NOV50a nucleic acid of 1196 nucleotides (also referred to as CG56771-01) encoding a Protein Arginine N-Methyltransferase 2-like protein is shown in Table 50A. An open reading frame was identified beginning with a ATG initiation codon at nucleotides 13-15 and ending with a TGA codon at nucleotides 1068-1070. The start and stop codons are shown in bold in Table 50A, and the 5′ and 3′ untranslated regions, if any, are underlined.

TABLE 50A
NOV50a nucleotide sequence.

(SEQ ID NO:187)

AGAGCGGCCAAGATGTCGCAGCCCAAGAAAAGAAAGCTTGAGTCGGGGGGCGGCGCCGAAGGAGGGGAGGGA
ACTGAAGAGGAAGATGGCGCGGAGCGGGAGGCGGCCCTGGAGCGACCCCGGAGGACTAAGCGGGAACGGGAC
CAGCTGTACTACGAGTGCTACTCGGACGTTTCGGTCCACGAGGAGATGATCGCGGACCGCGTCCGCACCGAT
GCCTACCGCCTGGGTATCCTTCGGAACTGGGCAGCACTGCGAGGCAAGACGGTACTGGACGTGGGCGCGGGC
ACCGGCATTCTGAGCATCTTCTGTGCCCAGGCCGGGGCCCGGCGCGTGTACGCGGTAGAGGCCAGCGCCATC
TGGCAACAGGCCCGGGAGGTGGTGCGGTTCAACGGGCTGGAGGACCGGGTGCACGTCCTGCCGGGACCAGTG
GAGACTGTAGAGTTGCCGGAACAGGTGGATGCCATCGTGAGCGAGTGGATGGGCTACGGACTCCTGCACGAG
TCCATGCTGAGCTCCGTCCTCCACGCGCGAACCAAGTGGCTGAAGGAGGGCGGTCTTCTCCTGCCGGCCTCC
GCCGAGCTCTTCATAGCCCCCATCAGCGACCAGATGCTGGAATGGCGCCTGGGCTTCTGGAGCCAGGTGAAG
CAGCACTATGGTGTGGACATGAGCTGCCTGGAGGGCTTCGCCACGCGCTGTCTCATGGGCTAGAGCTCTCCC
GCGCCGGCTTGCAGCAGGAGCTGGAGGCCGGAGTGGGCGGGCGCTTCCGCTGCAGCTGCTATGGCTCGGCGC
CCATGCATGGCTTTGCCATCTGGTTCCAGGTGACCTTCCCTGGAGGGGAGTCGGAGAAACCCCTGGTGCTGT
CCACCTCGCCTTTTCACCCGGCCACTCACTGGAAACAGGCGCTCCTCTACCTGAACGAGCCGGTGCAAGTGG
AGCAAGACACGGACGTTTCAGGAGAGATCACGCTGCTGCCCTCCCGGGACAACCCCCGTCGCCTGCGCGTGC
TGCTGCGCTACAAAGTGGGAGACCAGGAGGAGAAGACCAAAGACTTTGCCATGGAGGACTGAGCGTTGCCTT
TTCTCCCAGCTACCTCCCAAAGCAGCCTGACCTGCGTGGGAGAGGCGCCACTCGGAGATCGTTGTGCAGGGA
TTGTCCGGCGAGGACGTGCTGGCCCGGCCGCAGCGCTTTGCTCA

In a search of public sequence databases, the NOV50a nucleic acid sequence, located on chromosome 19, has 681 of 719 bases (94%) identical to a gb:GENBANK-ID:AK001421|acc:AK001421.1 mRNA from Homo sapiens (cDNA FLJ10559 fis, clone NT2RP2002618, weakly similar to Protein Arginine N-Methyltransferase 2 (EC 2.1.1.-)) (E=2.7e⁻¹³⁶).

The disclosed NOV50a polypeptide (SEQ ID NO:188) encoded by SEQ ID NO:187 has 375 amino acid residues and is presented in Table 50B using the one-letter amino acid code. Signal P, Psort and/or Hydropathy results predict that NOV50a has no signal peptide and is likely to be localized to the nucleus with a certainty of 0.7000. Alternatively, NOV50a may also localize to the microbody (peroxisome) with a certainty of 0.2641, to the mitochondrial matrix space with a certainty of 0.1000, or to the lysosome (lumen) with a certainty of 0.1000.

TABLE 50B
Encoded NOV50a protein sequence.

(SEQ ID NO:188)

MSQPKKRKLESGGGAEGGEGTEEEDGAEREAALERPRRTKRERDQLYYECYSDVSVHEEMIADRVRTDAYRL
GILRNWAALRGKTVLDVGAGTGILSIFCAQAGARRVYAVEASAIWQQAREVVRFNGLEDRVHVLPGPVETVE
LPEQVDAIVSEWMGYGLLHESMLSSVLHARTKWLKEGGLLLPASAELFIAPISDQMLEWRLGFWSQVKQHYG
VDMSCLEGFATRCLMGHSEIVVQGLSGEDVLARPQRFAQLELSRAGLEQELEAGVGGRFRCSCYGSAPMHGF
AIWFQVTFPGGESEKPLVLSTSPFHPATHWKQALLYLNEPVQVEQDTDVSGEITLLPSRDNPRRLRVLLRYK
VGDQEEKTKDFAMED

A search of sequence databases reveals that the NOV50a amino acid sequence has 316 of 316 amino acid residues (100%) identical to, and 316 of 316 amino acid residues (100%) similar to, the 316 amino acid residue ptnr:SPTREMBL-ACC:Q9NVR8 protein from Homo sapiens (Human) (cDNA FLJ10559 FIS, Clone NT2RP2002618, Weakly Similar To Protein Arginine N-Methyltransferase 2 (EC 2.1.1.-)) (E=1.7e⁻¹⁶⁹).

NOV50a is predicted to be expressed in at least lung, bronchus, kidney. This information was derived by determining the tissue sources of the sequences that were included in the invention including but not limited to SeqCalling sources, Public EST sources, Literature sources, and/or RACE sources.

NOV50b

In the present invention, the target sequence identified previously, NOV50a, was subjected to the exon linking process to confirm the sequence. PCR primers were designed by starting at the most upstream sequence available, for the forward primer, and at the most downstream sequence available for the reverse primer. In each case, the sequence was examined, walking inward from the respective termini toward the coding sequence, until a suitable sequence that is either unique or highly selective was encountered, or, in the case of the reverse primer, until the stop codon was reached. Such primers were designed based on in silico predictions for the full length cDNA, part (one or more exons) of the DNA or protein sequence of the target sequence, or by translated homology of the predicted exons to closely related human sequences sequences from other species. These primers were then employed in PCR amplification based on the following pool of human cDNAs: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea, uterus. Usually the resulting amplicons were gel purified, cloned and sequenced to high redundancy. The resulting sequences from all clones were assembled with themselves, with other fragments in CuraGen Corporation's database and with public ESTs. Fragments and ESTs were included as components for an assembly when the extent of their identity with another component of the assembly was at least 95% over 50 bp. In addition, sequence traces were evaluated manually and edited for corrections if appropriate. These procedures provide the sequence reported below, which is designated NOV50b. This differs from the previously identified sequence (NOV50a) at aminoacid position 15 A->G.

A disclosed NOV50b nucleic acid of 1165 nucleotides (also referred to as CG56771-02) encoding a Protein Arginine N-Methyltransferase 2-like protein is shown in Table 50C. An open reading frame was identified beginning with a ATG initiation codon at nucleotides 4-6 and ending with a TGA codon at nucleotides 1129-1131. The start and stop codons are shown in bold in Table 50C, and the 5′ and 3′ untranslated regions, if any, are underlined.

TABLE 50C
NOV50b nucleotide sequence.

(SEQ ID NO:189)

AAGATGTCGCAGCCCAAGAAAAGAAAGCTTGAGTCGGGGGGCGGCGGCGAAGGAGGGGAGGGAACTGAAGAG
GAAGATGGCGCGGAGCGGGAGGCGGCCCTGGAGCGACCCCGGAGGACTAAGCGGGAACGGGACCAGCTGTAC
TACGAGTGCTACTCGGACGTTTCGGTCCACGAGGAGATGATCGCGGACCGCGTCCGCACCGATGCCTACCGC
CTGGGTATCCTTCGGAACTGGGCAGCACTGCGAGGCAAGACGGTACTGGACGTGGGCGCGGGCACCGGCATT
CTGAGCATCTTCTGTGCCCAGGCCGGGGCCCGGCGCGTGTACGCGGTAGAGGCCAGCGCCATCTGGCAACAG
GCCCGGGAGGTGGTGCGGTTCAACGGGCTGGAGGACCGGGTGCACGTCCTGCCGGGACCAGTGGAGACTGTA
GAGTTGCCGGAACAGGTGGATGCCATCGTGAGCGAGTGGATGGGCTACGGACTCCTGCACGAGTCCATGCTG
AGCTCCGTCCTCCACGCGCGAACCAAGTGGCTGAAGGAGGGCGGTCTTCTCCTGCCGGCCTCCGCCGAGCTC
TTCATAGCCCCCATCAGCGACCAGATGCTGGAATGGCGCCTGGGCTTCTGGAGCCAGGTGAAGCAGCACTAT
GGTGTGGACATGAGCTGCCTGGAGGGCTTCGCCACGCGCTGTCTCATGGGCCACTCGGAGATCGTTGTGCAG
GGATTGTCCGGCGAGGACGTGCTGGCCCGGCCGCAGCGCTTTGCTCAGCTAGAGCTCTCCCGCGCCGGCTTG
GAGCAGGAGCTGGAGGCCGGAGTGGGCGGGCGCTTCCGCTGCAGCTGCTATGGCTCGGCGCCCATGCATGGC
TTTGCCATCTGGTTCCAGGTGACCTTCCCTGGAGGGGAGTCGGAGAAACCCCTGGTGCTGTCCACCTCGCCT
TTTCACCCGGCCACTCACTGGAAACAGGCGCTCCTCTACCTGAACGAGCCGGTGCAAGTGGAGCAAGACACG
GACGTTTCAGGAGAGATCACGCTGCTGCCCTCCCGGGACAACCCCCGTCGCCTGCGCGTGCTGCTGCGCTAC
AAAGTGGGAGACCAGGAGGAGAAGACCAAAGACTTTGCCATGGAGGACTGAGCGTTGCCTTTTCCCCCAGCT
ACCTCCCAAAGCA

In a search of public sequence databases, the NOV50b nucleic acid sequence, located on chromosome 19, has 1090 of 1091 bases (99%) identical to a gb:GENBANK-ID:AK001421|acc:AK001421.1 mRNA from Homo sapiens (cDNA FLJ10559 fis, clone NT2RP2002618, weakly similar to Protein Arginine N-Methyltransferase 2 (EC 2.1.1.-)) (E=2.2e⁻²⁴⁰).

The disclosed NOV50b polypeptide (SEQ ID NO:190) encoded by SEQ ID NO:189 has 375 amino acid residues and is presented in Table 50D using the one-letter amino acid code. Signal P, Psort and/or Hydropathy results predict that NOV50b has no signal peptide and is likely to be localized to the nucleus with a certainty of 0.7000. Alternatively, NOV50b may also localize to the microbody (peroxisome) with a certainty of 0.2766, to the mitochondrial matrix space with a certainty of 0.1000, or to the lysosome (lumen) with a certainty of 0.1000.

TABLE 50D
Encoded NOV50b protein sequence.

(SEQ ID NO:190)

MSQPKKRKLESGGGGEGGEGTEEEDGAEREAALERPRRTKRERDQLYYECYSDVSVHEEMIADRVRTDAYRL
GILRNWAALRGKTVLDVGAGTGILSIFCAQAGARRVYAVEASAIWQQAREVVRFNGLEDRVHVLPGPVETVE
LPEQVDAIVSEWMGYGLLHESMLSSVLHARTKWLKEGGLLLPASAELFIAPISDQMLEWRLGFWSQVKQHYG
VDMSCLEGFATRCLMGHSEIVVQGLSGEDVLARPQRFAQLELSRAGLEQELEAGVGGRFRCSCYGSAPMHGF
AIWFQVTFPGGESEKPLVLSTSPFHPATHWKQALLYLNEPVQVEQDTDVSGEITLLPSRDNPRRLRVLLRYK
VGDQEEKTKDFAMED

A search of sequence databases reveals that the NOV50b amino acid sequence has 316 of 316 amino acid residues (100%) identical to, and 316 of 316 amino acid residues (100%) similar to, the 316 amino acid residue ptnr:TREMBLNEW-ACC:AAH02729 protein from Homo sapiens (Human) (Hypothetical 35.2 Kda Protein) (E=1.8e⁻¹⁶⁹).

NOV50b is predicted to be expressed in at least lung, bronchus, kidney.

NOV50a also has homology to the amino acid sequences shown in the BLASTP data listed in Table 50E

TABLE 50E
BLAST results for NOV50a

| Gene Index/ | | Length | Identity | Positives | |
|---|---|---|---|---|---|
| Identifier | Protein/Organism | (aa) | (%) | (%) | Expect |
| gi\|15822652\|gb\|AAK85733.1\| | arginine | 375 | 374/375 | 374/375 | 0.0 |
| (AY043278) | methyltransferase | | (99%) | (99%) | |
| | 6 [Homo sapiens] | | | | |
| gi\|8922515\|ref\|NP_060607.1\| | hypothetical | 316 | 316/316 | 316/316 | 0.0 |
| (NM_018137) | protein FLJ10559 | | (100%) | (100%) | |
| | [Homo sapiens] | | | | |
| gi\|9293956\|dbj\|BAB01859.1\| | protein arginine | 399 | 148/317 | 193/317 | 5e−66 |
| (AP000383) | N- | | (46%) | (60%) | |
| | methyltransferase- | | | | |
| | like | | | | |
| | protein [Arabidopsis | | | | |
| | thaliana] | | | | |
| gi\|15231011\|ref\|NP_{—} | arginine | 409 | 143/310 | 185/310 | 1e−65 |
| 188637.1\| | methyltransferase, | | (46%) | (59%) | |
| (NC_003074) | putative | | | | |
| | [Arabidopsis | | | | |
| | thaliana] | | | | |
| gi\|15233606\|ref\|NP_{—} | arginine | 390 | 135/365 | 201/365 | 4e−58 |
| 194680.1\| | methyltransferase | | (36%) | (54%) | |
| (NC_003075) | (pam1) | | | | |
| | [Arabidopsis | | | | |

The homology between these and other sequences is shown graphically in the ClustalW analysis shown in Table 50F. In the ClustalW alignment of the NOV50 protein, as well as all other ClustalW analyses herein, the black outlined amino acid residues indicate regions of conserved sequence (i.e., regions that may be required to preserve structural or functional properties), whereas non-highlighted amino acid residues are less conserved and can potentially be altered to a much broader extent without altering protein structure or function.

[image]

[image]

Table 49G lists the domain description from DOMAIN analysis results against NOV50. This indicates that the NOV50 sequence has properties similar to those of other proteins known to contain this domain.

TABLE 50G
Domain Analysis of NOV50

gnl|Pfam|Pfam01209, Ubie_methyltran, ubiE/COQ5 methyltransferase
family. (SEQ ID NO:849)
CD-Length = 237 residues, 63.3% aligned
Score = 35.0 bits (79), Expect = 0.008

| | | | |
|---|---|---|---|
| NOV50: | 40 | KRERDQLYYECYSDVSVHEEMIADRVRTDAYRLGILRNW---------AALRGKTVLDVG | 90 |
| | | \| \|++\| + ++ \|+ +++ \| \|\| \| \| \|\|\| \|\|\| | |
| Sbjct: | 5 | KEEKEQKVHHVFASVAKKYDLM-----NDVMSFGIHRLWKDHFTMKLMGPKRGKKFLDVA | 59 |
| NOV50: | 91 | AGTGILSI-FCAQAGAR-RVYAVEASA-IWQQAREVVRFNGLEDRVHVLPGPVETVELPE | 147 |
| | | \|\|\| ++ \|\| +\| ++ + + + ++ + \|+ \| \| \|\|\| | |
| Sbjct: | 60 | GGTGDIAFRLLRHAGTSGKVVVLDINENMLKVGKKRAE-EEGKIRIEWLCANAE--ELPF | 116 |
| NOV50: | 148 | QVDAIVSEWMGYGLLHESMLSSVLHARTKWLKEGGLLL 185 | |
| | | + + + +\|+ + + \|\| + \|\| \|\| \|+ | |
| Sbjct: | 117 | EDNTFDLVTISFGIRNFTDYLKVLREAFRVLKPGGQLV 154 | |

Methyl transfer from S-Adenosyl-L-methionine (SAM) to either nitrogen, oxygen or carbon atoms is frequently employed in diverse organisms ranging from bacteria to plants and mammals. The reaction is catalyzed by methyltransferases (MTases) and modifies DNA, RNA, proteins and small molecules like catechol. The catalytic domain of SAM-MTases is of the alpha/beta type with a central mixed beta-sheet around which several alpha-helices are arranged. Topologically it can be divided into two halves. The first half, formed by beta1-alphaA-beta2-alphaB-beta3-alphaC, is mainly responsible for SAM binding. The second half, beta4-alphaD-beta5-alphaE-beta6-beta7, is primarily responsible for catalysis. According to the sequential order of these two sites, the SAM-MTases can be divided into three families Protein arginine methylation has been implicated in signal transduction, nuclear transport and transcription regulation. Protein arginine methyltransferases (PRMTs) mediate the AdoMet-dependent methylation of many proteins, including many RNA binding proteins involved in various aspects of RNA processing and/or transport.

The bulk of methylated arginine residues in eukaryotic cells are found in heterogeneous nuclear ribonucleoproteins (hnRNPs), RNA-binding proteins that play essential roles in the metabolism of nuclear pre-mRNA. Lin et al. (1996) identified a rat cDNA encoding PRMT1 (protein-arginine N-methyltransferase 1; EC 2.1.1.23). Recombinant PRMT1 methylated histones and hnRNPA1 (164017) in vitro. By using a yeast 2-hybrid screen to identify proteins that interact with the intracytoplasmic domain of the interferon-alpha/beta receptor-1 (IFNAR1; 107450), Abramovich et al. (1997) identified a human cDNA encoding a protein that was nearly identical to PRMT1. The deduced 361-amino acid protein was designated IR1B4 for ‘interferon receptor-1-bound protein 4.’ Epitope-tagged IR1B4 bound the IFNAR1 intracytoplasmic domain in vitro. Antibodies against IFNAR1 coimmunoprecipitated a methyltransferase activity from human cell extracts. An antisense oligonucleotide strongly reduced methyltransferase activity in human cells, and caused them to become more resistant to growth inhibition by interferon. Abramovich et al. (1997) concluded that protein methylation, like phosphorylation, may be an important signaling mechanism for certain cytokine receptors. Scott et al. (1998) identified HRMT1L2 transcripts with variable 5-prime ends that encode 3 protein variants with different N-terminal regions. Rat PRMT1 and HRMT1L2 variant 2 (v.2) share 95% sequence identity, but diverge at their N termini. The amino acid sequences of HRMT1L2 and HRMT1L1 (601961) are 27% identical. Recombinant protein methylated human hnRNPA1 and a yeast hnRNP in vitro. The HRMT1L2 gene complemented mutations in the yeast hnRNP methyltransferase gene HMT1. Northern blot analysis revealed that HRMT1L2 is expressed as a predominant 1.4-kb mRNA in various adult and fetal tissues. Additional larger and smaller bands were observed in some tissues.

The disclosed NOV50 nucleic acid of the invention encoding a Protein Arginine N-Methyltransferase 2-like protein includes the nucleic acid whose sequence is provided in Table 50A, 50C, or a fragment thereof. The invention also includes a mutant or variant nucleic acid any of whose bases may be changed from the corresponding base shown in Table 50A or 50C while still encoding a protein that maintains its Protein Arginine N-Methyltransferase 2-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 6 percent of the bases may be so changed.

The disclosed NOV50 protein of the invention includes the Protein Arginine N-Methyltransferase 2-like protein whose sequence is provided in Table 50B or 50D. The invention also includes a mutant or variant protein any of whose residues may be changed from the corresponding residue shown in Table 50B or 50D while still encoding a protein that maintains its Protein Arginine N-Methyltransferase 2-like activities and physiological functions, or a functional fragment thereof. In the mutant or variant protein, up to about 63 percent of the residues may be so changed.

The invention further encompasses antibodies and antibody fragments, such as F_{ab} or (F_{ab})₂, that bind immunospecifically to any of the proteins of the invention.

The above disclosed information suggests that this Protein Arginine N-Methyltransferase 2-like protein (NOV50) is a member of a “Protein Arginine N-Methyltransferase 2 family”. Therefore, the NOV50 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV50 nucleic acids and proteins of the invention are useful in potential therapeutic applications implicated in systemic lupus erythematosus, autoimmune disease, asthma, emphysema, scleroderma, allergy, ards, diabetes, autoimmune disease, renal artery stenosis, interstitial nephritis, glomerulonephritis, polycystic kidney disease, renal tubular acidosis, IGA nephropathy, hypercalceimia, Lesch-Nyhan syndrome, and/or other diseases and pathologies.

NOV50 nucleic acids and polypeptides are further useful in the generation of antibodies that bind immuno-specifically to the novel NOV50 substances for use in therapeutic or diagnostic methods. These antibodies may be generated according to methods known in the art, using prediction from hydrophobicity charts, as described in the “Anti-NOVX Antibodies” section below. The disclosed NOV50 protein has multiple hydrophilic regions, each of which can be used as an immunogen. These novel proteins can be used in assay systems for functional analysis of various human disorders, which will help in understanding of pathology of the disease and development of new drug targets for various disorders.

NOV51

A disclosed NOV51 nucleic acid of 984 nucleotides (also referred to as CG56759-01) encoding a Olfactory Receptor-like protein is shown in Table 51A. An open reading frame was identified beginning with a ATG initiation codon at nucleotides 9-11 and ending with a TAA codon at nucleotides 954-956. The start and stop codons are shown in bold in Table 51A, and the 5′ and 3′ untranslated regions, if any, are underlined.

TABLE 51A
NOV51 nucleotide sequence.

(SEQ ID NO:191)

CTGGCCTAATGAATGTCTCTGAGCCAAATTCCAGCTTTGCTTTTGTAAATGAATTTATACTCCAAGGTTTCT
CTTGTGAGTGGACAATTCAGATCTTCCTCTTCTCACTCTTTACTACAATATATGCACTGACTATAACAGGGA
ATGGAGCCATTGCTTTTGCCCTGTGGTGTGACCGGCGACTTCACACTCCCATGTACATGTTCCTGGGAGATT
TCTCCTTTTTAGAGATATGGTATGTCTTTTCTACAGTTCCCAAGATGTTGGTCAACTTCCTTTCAGAGAAAA
CAAACATCTCCTTTGCTGGATGTTTTCTCCAGTTTTATTTCTTCTTCTCTTTGGGTACATCAGAATGCTTGC
TTTTGACTGTGATGGCCTTTGATCAGTACCTTGCTATCTGCCGTCCCTTGCACTATCCTAATATCATGACTG
GGCATCTCTGTGCCAAACTGGTCATACTGTGCTGGGTTTGTGGATTTCTGTGGTTCCTGATCCCCATTGTTC
TCATCTCTCAGATGCCCTTCTGTGGCCCAAACATTATTGACCATGTTGTGTGTGACCCAGGGCCACTATTTG
CATTGGATTGTGTTTCTGCCCCAAGAATCCAACTGTTTTGCTACACTCTAAACTCATTAGTTATTTTTGGTA
ACTTCCTCTTTATTATTGGATCCTATACTATTGTCCTGAAAGCTGTGTTGGGTACACCTTCAAGCACTGGGA
GACATAAGGCCTTCTCTACCTGTGGGTCTCATTTGGCTGTGGTATCACCGTGCTATGGCTCTCTTATGGTCA
TGTATGTGAGCCCAGGACTCGGACATTCTACGGGGATGCAGAAAATTGTAACTTTGTTCTATGCTATGGTGA
CCCCACTCTTCAATCCCCTTATCTATAGCCTCCAGAATAAGGAGATAAAGGCAGCCCTGAGGAAAGTTCTGG
GGAGTTCCAACATAATCTAAGGCATATTAGATTATTCCTCCATGATCA

In a search of public sequence databases, the NOV51 nucleic acid sequence, located on chromosome 22, has 967 of 984 bases (98%) identical to a gb:GENBANK-ID:AP000534|acc:AP000534.1 mRNA from Homo sapiens (genomic DNA, chromosome 22q11.2, Cat Eye Syndrome region, clone:c23H5) (E=2.9e⁻²⁰⁸).

The disclosed NOV51 polypeptide (SEQ ID NO:192) encoded by SEQ ID NO:191 has 315 amino acid residues and is presented in Table 51B using the one-letter amino acid code. Signal P, Psort and/or Hydropathy results predict that NOV51 has a signal peptide and is likely to be localized to the plasma mambrane with a certainty of 0.6000. Alternatively, NOV51 may also localize to the Golgi body with a certainty of 0.4000, to the endoplasmic reticulum (membrane) with a certainty of 0.3000, or to the microbody (peroxisome) with a certainty of 0.3000. The most likely cleavage site for NOV51 is between positions 40 and 41: IYA-LT.

TABLE 51B
Encoded NOV51 protein sequence.

(SEQ ID NO:192)

MNVSEPNSSFAFVNEFILQGFSCEWTIQIFLFSLFTTIYALTITGNGAIAFALWCDRRLHTPMYMFLGDFSF
LEIWYVFSTVPKMLVNFLSEKTNISFAGCFLQFYFFFSLGTSECLLLTVMAFDQYLAICRPLHYPNIMTGHL
CAKLVILCWVCGFLWFLIPIVLISQMPFCGPNIIDHVVCDPGPLFALDCVSAPRIQLFCYTLNSLVIFGNFL
FIIGSYTIVLKAVLGTPSSTGRHKAFSTCGSHLAVVSPCYGSLMVMYVSPGLGHSTGMQKIVTLFYAMVTPL
FNPLIYSLQNKEIKAALRKVLGSSNII

A search of sequence databases reveals that the NOV51 amino acid sequence has 191 of 314 amino acid residues (60%) identical to, and 226 of 314 amino acid residues (71%) similar to, the 324 amino acid residue ptnr:SPTREMBL-ACC:Q9WU86 protein from Mus musculus (Mouse) (Odorant Receptor S1) (E=7.0e⁻¹⁰⁰).

NOV51 is predicted to be expressed in at least Apical microvilli of the retinal pigment epithelium, arterial (aortic), basal forebrain, brain, Burkitt lymphoma cell lines, corpus callosum, cardiac (atria and ventricle), caudate nucleus, CNS and peripheral tissue, cerebellum, cerebral cortex, colon, cortical neurogenic cells, endothelial (coronary artery and umbilical vein) cells, palate epithelia, eye, neonatal eye, frontal cortex, fetal hematopoietic cells, heart, hippocampus, hypothalamus, leukocytes, liver, fetal liver, lung, lung lymphoma cell lines, fetal lymphoid tissue, adult lymphoid tissue, Those that express MHC II and III nervous, medulla, subthalamic nucleus, ovary, pancreas, pituitary, placenta, pons, prostate, putamen, serum, skeletal muscle, small intestine, smooth muscle (coronary artery in aortic) spinal cord, spleen, stomach, taste receptor cells of the tongue, testis, thalamus, and thymus tissue. This information was derived by determining the tissue sources of the sequences that were included in the invention including but not limited to SeqCalling sources, Public EST sources, Literature sources, and/or RACE sources.

NOV51 also has homology to the amino acid sequences shown in the BLASTP data listed in Table 51C

TABLE 51C
BLAST results for NOV51

| Gene Index/ | | Length | Identity | Positives | |
|---|---|---|---|---|---|
| Identifier | Protein/Organism | (aa) | (%) | (%) | Expect |
| gi\|15293807\|gb\|AAK95096.1\| | olfactory | 217 | 205/217 | 208/217 | 4e−99 |
| (AF399611) | receptor [Homo | | (94%) | (95%) | |
| | sapiens] | | | | |
| gi\|9938010\|ref\|NP_064684.1\| | odorant receptor | 324 | 191/314 | 226/314 | 5e−90 |
| (NM_020288) | S1 gene [Mus | | (60%) | (71%) | |
| | musculus] | | | | |
| gi\|17476501\|ref\|XP_{—} | similar to | 1056 | 134/293 | 181/293 | 2e−66 |
| 063251.1\| | OLFACTORY | | (45%) | (61%) | |
| (XM_063251) | RECEPTOR-LIKE | | | | |
| | PROTEIN F6 (H. | | | | |
| | sapiens) [Homo | | | | |
| | sapiens] | | | | |
| gi\|15293805\|gb\|AAK95095.1\| | olfactory | 217 | 142/217 | 163/217 | 2e−65 |
| (AF399610) | receptor [Homo | | (65%) | (74%) | |
| | sapiens] | | | | |
| gi\|17476700\|ref\|XP_{—} | similar to | 195 | 131/189 | 156/189 | 2e−64 |
| 063315.1\| | odorant receptor | | (69%) | (82%) | |
| (XM_063315) | S1 gene (H. | | | | |
| | sapiens) [Homo | | | | |
| | sapiens] | | | | |

The homology between these and other sequences is shown graphically in the ClustalW analysis shown in Table 51D. In the ClustalW alignment of the NOV51 protein, as well as all other ClustalW analyses herein, the black outlined amino acid residues indicate regions of conserved sequence (i.e., regions that may be required to preserve structural or functional properties), whereas non-highlighted amino acid residues are less conserved and can potentially be altered to a much broader extent without altering protein structure or function.

[image]

[image]

[image]

[image]

Table 51E lists the domain descriptions from DOMAIN analysis results against NOV51. This indicates that the NOV51 sequence has properties similar to those of other proteins known to contain this domain.

TABLE 51E
Domain Analysis of NOV51

gnl|Pfam|pfam00001, 7tm_1, 7 transmembrane receptor (rhodopsin
family). (SEQ ID NO:810)
CD-Length = 254 residues, 100.0% aligned
Score = 105 bits (262), Expect = 4e−24

| | | | |
|---|---|---|---|
| NOV51: | 45 | GNGAIAFALWCDRRLHTPMYMFLGDFSFLEIWYVFSTVPKMLVNFLSEKTNISFAGCFLQ | 104 |
| | | \|\| + + ++\| \|\| +\|\| + + ++ ++ + \| \| + \| \| \| | |
| Sbjct: | 1 | GNLLVILVILRTKKLRTPTNIFLLNLAVADLLFLLTLPPWALYYLVGGDWVFGDALCKLV | 60 |
| NOV51: | 105 | FYFFFSLGTSECLLLTVMAFDQYLAICRPLHYPNIMTGHLCAKLVILCWVCGFLWFLIPI | 164 |
| | | \| \| + \|\|\|\| ++ \|+\|\|\|\| \|\| \| \| \| \|++\| \|\| \| \| \|+ | |
| Sbjct: | 61 | GALFVVNGYASILLLTAISIDRYLAIVHPLRYRRIRTPRRAKVLILLVWVLALLLSLPPL | 120 |
| NOV51: | 165 | VLISQMPFCGPNIIDHVVCDPGPLFALDCVSAPRIQLFCYTLNSLVIFGNFLFIIGSYTI | 224 |
| | | + \| ++ \| \| + \| \| +++ + | |
| Sbjct: | 121 | LFSWLRTVEEGNTTVCLIDFPEESVKRSYVLLSTLVGFVLPLLVILVCYTRILRTLRKRA | 180 |
| NOV51: | 225 | VLKAVLGTPSSTGRHKAFSTCGSHLAVVSPCYGSLMVMYVSP----GLGHSTGMQKIVTL | 280 |
| | | + \| \|\|+ \| \| + \| +\|+ + + ++\|\| | |
| Sbjct: | 181 | RSQRSLKRRSSSERKAAKMLLVVVVVFVLCWLPYHIVLLLDSLCLLSIWRVLPTALLITL | 240 |
| NOV51: | 281 | FYAMVTPLFNPLIY 294 | |
| | | + \| \| \|\|+\|\| | |
| Sbjct: | 241 | WLAYVNSCLNPIIY 254 | |

G-Protein Coupled Receptor (GPCRs) have been identified as an extremely large family of protein receptors in a number of species. At the phylogenetic level they can be classified into four major subfamilies. These receptors share a seven transmembrane domain structure with many neurotransmitter and hormone receptors. They are likely to be involved in the recognition and transduction of various signals mediated by G-Proteins, hence their name G-Protein Coupled Receptors. The human GPCR genes are generally intron-less and belong to four gene subfamilies, displaying great sequence variability. These genes are dominantly expressed in olfactory epithelium.

Olfactory receptors (ORs) have been identified as extremely large family of GPCRs in a number of species. As members of the GPCR family, these receptors share a seven transmembrane domain structure with many neurotransmitter and hormone receptors, and are likely to underlie the recognition and G-protein-mediated transduction of odorant signals. Like GPCRs, the ORs they can be expressed in a variety of tissues where they are thought to be involved in recognition and transmission of a variety of signals. The human OR genes are typically intron-less and belong to four different gene subfamilies, displaying great sequence variability. These genes are dominantly expressed in olfactory epithelium.

The disclosed NOV51 nucleic acid of the invention encoding a Olfactory Receptor-like protein includes the nucleic acid whose sequence is provided in Table 51A or a fragment thereof. The invention also includes a mutant or variant nucleic acid any of whose bases may be changed from the corresponding base shown in Table 51A while still encoding a protein that maintains its Olfactory Receptor-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 2 percent of the bases may be so changed.

The disclosed NOV51 protein of the invention includes the Olfactory Receptor-like protein whose sequence is provided in Table 51B. The invention also includes a mutant or variant protein any of whose residues may be changed from the corresponding residue shown in Table 51B while still encoding a protein that maintains its Olfactory Receptor-like activities and physiological functions, or a functional fragment thereof. In the mutant or variant protein, up to about 54 percent of the residues may be so changed.

The invention further encompasses antibodies and antibody fragments, such as F_{ab} or (F_{ab})₂, that bind immunospecifically to any of the proteins of the invention.

The above disclosed information suggests that this Olfactory Receptor-like protein (NOV51) is a member of a “Olfactory Receptor family”. Therefore, the NOV51 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV51 nucleic acids and proteins of the invention are useful in potential therapeutic applications implicated in developmental diseases, MHCII and III diseases (immune diseases), Taste and scent detectability Disorders, Burkitt's lymphoma, Corticoneurogenic disease, Signal Transduction pathway disorders, Retinal diseases including those involving photoreception, Cell Growth rate disorders; Cell Shape disorders, Feeding disorders; control of feeding; potential obesity due to over-eating; potential disorders due to starvation (lack of apetite), noninsulin-dependent diabetes mellitus (NIDDM1), bacterial, fungal, protozoal and viral infections (particularly infections caused by HIV-1 or HIV-2), pain, cancer (including but not limited to Neoplasm; adenocarcinoma; lymphoma; prostate cancer; uterus cancer), anorexia, bulimia, asthma, Parkinson's disease, acute heart failure, hypotension, hypertension, urinary retention, osteoporosis, Crohn's disease; multiple sclerosis; and Treatment of Albright Hereditary Ostoeodystrophy, angina pectoris, myocardial infarction, ulcers, asthma, allergies, benign prostatic hypertrophy, and psychotic and neurological disorders, including anxiety, schizophrenia, manic depression, delirium, dementia, severe mental retardation. Dentatorubro-pallidoluysian atrophy(DRPLA) Hypophosphatemic rickets, autosomal dominant (2) Acrocallosal syndrome and dyskinesias, such as Huntington's disease or Gilles de la Tourette syndrome and/or other pathologies and disorders of the like. The polypeptides can be used as immunogens to produce antibodies specific for the invention, and as vaccines. They can also be used to screen for potential agonist and antagonist compounds. For example, a cDNA encoding the OR-like protein may be useful in gene therapy, and the OR-like protein may be useful when administered to a subject in need thereof. By way of nonlimiting example, the compositions of the present invention will have efficacy for treatment of patients suffering from bacterial, fungal, protozoal and viral infections (particularly infections caused by HIV-1 or HIV-2), pain, cancer (including but not limited to Neoplasm; adenocarcinoma; lymphoma; prostate cancer; uterus cancer), anorexia, bulimia, asthma, Parkinson's disease, acute heart failure, hypotension, hypertension, urinary retention, osteoporosis, Crohn's disease; multiple sclerosis; and Treatment of Albright Hereditary Ostoeodystrophy, angina pectoris, myocardial infarction, ulcers, asthma, allergies, benign prostatic hypertrophy, and psychotic and neurological disorders, including anxiety, schizophrenia, manic depression, delirium, dementia, severe mental retardation and dyskinesias, such as Huntington's disease or Gilles de la Tourette syndrome, and/or other diseases and pathologies.

NOV51 nucleic acids and polypeptides are further useful in the generation of antibodies that bind immuno-specifically to the novel NOV51 substances for use in therapeutic or diagnostic methods. These antibodies may be generated according to methods known in the art, using prediction from hydrophobicity charts, as described in the “Anti-NOVX Antibodies” section below. The disclosed NOV51 protein has multiple hydrophilic regions, each of which can be used as an immunogen. These novel proteins can be used in assay systems for functional analysis of various human disorders, which will help in understanding of pathology of the disease and development of new drug targets for various disorders.

NOV52

A disclosed NOV52 nucleic acid of 3828 nucleotides (also referred to as CG56731-01) encoding a H326-like protein is shown in Table 52A. An open reading frame was identified beginning with a ATG initiation codon at nucleotides 177-179 and ending with a TAA codon at nucleotides 1968-1970. The start and stop codons are shown in bold in Table 52A, and the 5′ and 3′ untranslated regions, if any, are underlined.

TABLE 52A
NOV52 nucleotide sequence.

(SEQ ID NO:193)

CTCTTAGCGCTCAGGTCTTTTCCTTCCGCCGACCCGAAGTCATCGCTGGGAGTACTGGTTGCCCTTTCCTCA
GTCCTTCAGTGAATCTACAGAGCCTATTTCCTCAGGAGCCTCAGCCTGGTCCTTACTTCAGTGATAAAAGGA
GGAAAGGCTGGCTACAGCAAACATCATTCAAGATGTCCAGCAAAGGGAGCAGCACAGATGGCAGAACAGACT
TAGCTAATGGAAGCCTGTCTAGCAGTCCAGAGGAGATGTCTGGAGCTGAAGAGGGGAGGGAGACATCCTCAG
GCATTGAAGTGGAGGCCTCAGACCTGAGTTTGAGCTTGACTGGGGATGATGGTGGCCCCAACCGCACCAGCA
CAGAAAGTCGAGGCACAGACACAGAGAGCTCAGGTGAAGATAAGGACTCTGACAGCATGGAGGACACTGGTC
ATTACTCCATTAATGATGAAAATCGAGTCCATGACCGCTCAGAGGAAGAGGAAGAGGAGGAAGAAGAGGAGG
AAGAAGAGCAGCCTCGGCGCCGTGTACAGCGCAAGCGGGCTAACCGTGACCAGGACTCATCAGATGATGAGC
GGGCCCTAGAGGACTGGGTGTCCTCAGAAACATCAGCTCTACCCCGACCTCGCTGGCAAGCCCTCCCTGCCC
TTCGGGAGCGGGAGCTGGGTTCAAGTGCCCGCTTTGTCTATGAGGCCTGTGGGGCAAGAGTCTTTGTGCAGC
GTTTCCGCCTGCAGCATGGGCTTGAGGGCCATACTGGTTGTGTCAATACCCTGCACTTTAACCAGCGCGGCA
CCTGGCTGGCCAGTGGCAGCGATGACCTGAAGGTGGTGGTGTGGGATTGGGTACGGCGGCAGCCAGTACTGG
ACTTTGAGAGTGGCCACAAAAGTAATGTGTTCCAGGCCAAGTTTCTTCCTAACAGTGGTGATTCTACTCTGG
CCATGTGTGCCCGTGACGGGCAGGTTCGAGTAGCAGAACTGTCTGCCACACAGTGTTGCAAGAATACAAAAC
GTGTGGCCCAGCACAAGGGAGCGTCCCACAAGTTGGCACTGGAACCAGACTCTCCCTGTACGTTCTTATCTG
CAGGTGAAGATGCAGTTGTTTTCACCATTGACCTGAGACAAGACCGCCCAGCGTCGAAACTGGTGGTGACAA
AAGAGAAAGAGAAGAAAGTGGGGCTGTATACGATCTATGTGAATCCTGCCAATACCCACCAGTTTGCAGTGG
GTGGACGAGATCAGTTTGTAAGGATTTATGACCAGAGGAAAATTGATGAGAATGAGAACAATGGAGTACTCA
AGAAGTTCTGTCCTCATCACCTGGTGAACAGTGAGTCCAAAGCAAACATCACCTGTCTTGTGTACAGCCACG
ACGGCACAGAGCTCCTGGCCAGTTACAATGATGAAGACATTTACCTCTTCAACTCCTCTCACAGTGATGGGG
CCCAGTATGTTAAGAGATACAAGGGCCACAGAAATAATGCCACAGTAAAAGGCGTCAATTTCTATGGCCCCA
AGAGTGAGTTTGTGGTGAGCGGTAGTGACTGTGGGCACATCTTCCTCTGGGAGAAATCATCCTGCCAGATTA
TTCAGTTCATGGAGGGGGACAAGGGAGGCGTGGTAAACTGTCTTGAGCCCCACCCTCACCTGCCTGTGCTGG
CAACCAGTGGCCTAGACCATGATGTGAAGATCTGGGCACCCACAGCTGAAGCTTCCACTGAGCTGACAGGGT
TAAAAGATGTGATTAAGAAGAACAAGCGGGAGCGGGATGAAGATAGCTTGCACCAAACTGACCTGTTTGATA
GTCACATGCTGTGGTTCCTTATGCATCACCTGAGACAGAGACGCCATCACCGGCGCTGGCGAGAACCTGGGG
TTGGGGCCACAGACGCGGACTCTGATGAGTCTCCCAGCTCCTCAGACACATCGGACGAGGAGGAGGGCCCTG
ACCGGGTGCAGTGCATGCCATCTTGAGGCCTCATACCTAGGTGGGGCAGGCTGGGGCTGCCAACCTGATCCT
GCCTGGGCAACCCTTTCCTGTCCCAGGCCCTACATTCAGCAGAAACGCACTTTGGACTTTTTGCTTTAGATA
AAAGAAAGACATCCCAGGAGAAGGACAAACCAGAGGAGTGAACCAACAAAGAGTACCTAGGAATGGGAGTTG
AGCCTGGAATGGGCTCCATGGAGAGGTGCATAGGACTCGGCAGAAATGGCCTCTCCCCAAAGCCTCTTTTTG
AGAGGAGAGGGAAGCCTATTTGTTAACTGGTTTGGGATAGGGAATGGGGTTTCTTTTTCTTTAATCTCCCTT
GTTTCTTGGGCTGGGGGAGGGGTGGGGGGAACAACTGGCTATTCAGTACCAAGGGGCCAGAGTGGAGGGTAG
GAGTGCCACTCTCTCTTTGGTTTAGGTTTTTGACCTTTTCTTCCTTTGTTTTTTAAAAGTTTATGACAGTTN
CTCCCNNNACCCCACAACCCCATCCCAGAATCCTATTTTCCTGGGAAGTCCTTAAAGCCCCTAACCATCCCA
CACTCTTCACTTTCCTTTCCACCTTATTCATTCTCTGTACTTACCACAGTATTTTGCACTTGATTACATATC
CTTCATCTCTTCTCTTCATCCCATCACCCCCTAAATAGGTCAGGTGAGGGAGGCTGGGAAGAGGTGGGAGGA
GGGGAGAAGTGAAGGAAGATAGGAAGGATATTACCTCTTCTGTTATTTTTTTAAGAAACATTGTTTGGTGGC
AGCAATCTCCCTGTCCCTATCACTGTTAGAGGCCTAATTTTATATCTATAAATATATTAAAAAGCAAGTCAA
ACTTGGATGTATCAAGGTAAAATTATTGTCAAAGTTTAAATACCTATATATTCTCTGAATGCAATAAAGGGA
CTTAAGAGTGAACAAGAGTAATGGTGTGGAAGTGACACCTGGGGTCAGTTTACCTCTGTGTATGGTCACTAG
AGATTGGGACTTACCCTTTAGGTTTTAGGAGGCTTGAGAATGGAAGGATCCTCATTTCTGCCCTTCCTGGTT
CCCTGCTTTGGTGTAGGGGTTGGGAAAAACAGGAAATTCCTCTCAGCTCTGCCTCAGATCTCCTACCTCTCC
TTAAGTCTTGTAGGGGGTTCCAAGGATGGCTCTTCTAACCAGAGGCTGGCCTGTCTTTAAAACTTAACTACT
TTAGGGTGGTGCCACCACTGCAGACTATTGTGGTACTTTGTGACAGAAGACATGTACACACACACCACACAC
ATACATACACACTCTCTCACTCTGTCTCTCTTACCTTTAGCTGCTTGATCATTAAGCCATCCAACTTCATGC
CAGTTCCCTTCTTTATAGAAGAGTGAAGGGAAAGACTTCCTGGGTTTGACTTAAACCTTGTCCACCTTCTTG
ATATTTTAGGATTGAGGAATAAAGTCATTAATCTAAGGAACTGATTACAGTGGCTGGAGCTTGGGCACTTGT
CTTATCACTGGTCACTGAGTCTGAAAGTCCCAGNTGAATTCTTGCCCTTAAGTGCTTTTGCTGCTATTTTTT
TGCCCCCAGTTCCACAAGATCCAACCAAGAATTCTGTATCCTGGCAACAGTCAGATTCTTCTAAATCAGCCA
GCAAGAGGGNAAAGAGTGAGAGATGGTATTCCCAGATCATTCTTCCTCCTGCCCCTTTCCCAGCAGCTCTAG
ACCAGATGTTGGCTGCTGTACTTACTCCCTGAGGTAGGGAATGTGTGGTGATCGAGTGGTCTGTGTTCCTAT
TGCTGGTGGGGTGATAGGGTGGGCTAAAAACCATGCACTCTGGAATTTGTTGTATTTTCTCCCAGTAAAGCT
TTTCTTCTCCCG

In a search of public sequence databases, the NOV52 nucleic acid sequence, located on chromosome 22, has 3818 of 3828 bases (99%) identical to a gb:GENBANK-ID:HSU06631|acc:U06631.1 mRNA from Homo sapiens (Human (H326) mRNA, complete cds) (E=0.0).

The disclosed NOV52 polypeptide (SEQ ID NO:194) encoded by SEQ ID NO:193 has 597 amino acid residues and is presented in Table 52B using the one-letter amino acid code. Signal P, Psort and/or Hydropathy results predict that NOV52 has no signal peptide and is likely to be localized to the microbody (peroxisome) with a certainty of 0.3000. Alternatively, NOV52 may also localize to the nucleus with a certainty of 0.3000, to the mitochondrial matrix space with a certainty of 0.1000, or to the lysosome (lumen) with a certainty of 0.1000.

TABLE 52B
Encoded NOV52 protein sequence.

(SEQ ID NO:194)

MSSKGSSTDGRTDLANGSLSSSPEEMSGAEEGRETSSGIEVEASDLSLSLTGDDGGPNRTSTESRGTDTESS
GEDKDSDSMEDTGHYSINDENRVHDRSEEEEEEEEEEEEEQPRRRVQRKRANRDQDSSDDERALEDWVSSET
SALPRPRWQALPALRERELGSSARFVYEACGARVFVQRFRLQHGLEGHTGCVNTLHFNQRGTWLASGSDDLK
VVVWDWVRRQPVLDFESGHKSNVFQAKFLPNSGDSTLAMCARDGQVRVAELSATQCCKNTKRVAQHKGASHK
LALEPDSPCTFLSAGEDAVVFTIDLRQDRPASKLVVTKEKEKKVGLYTIYVNPANTHQFAVGGRDQFVRIYD
QRKIDENENNGVLKKFCPHHLVNSESKANITCLVYSHDGTELLASYNDEDIYLFNSSHSDGAQYVKRYKGHR
NNATVKGVNFYGPKSEFVVSGSDCGHIFLWEKSSCQIIQFMEGDKGGVVNCLEPHPHLPVLATSGLDHDVKI
WAPTAEASTELTGLKDVIKKNKRERDEDSLHQTDLFDSHMLWFLMHHLRQRRHHRRWREPGVGATDADSDES
PSSSDTSDEEEGPDRVQCMPS

A search of sequence databases reveals that the NOV52 amino acid sequence has 588 of 597 amino acid residues (98%) identical to, and 589 of 597 amino acid residues (98%) similar to, the 597 amino acid residue ptnr:SPTREMBL-ACC:Q12839 protein from Homo sapiens (Human) (H326) (E=0.0).

NOV52 is predicted to be expressed in at least adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea, uterus, Amnion, Appendix, Bone, Bronchus, Brown adipose, Cervix, Chorionic Villus, Colon, Coronary Artery, Dermis, Epidermis, Foreskin, Hair Follicles, Hypothalamus, Kidney Cortex, Liver, Lung, Lung Pleura, Lymph node, Lymphoid tissue, Muscle, Ovary, Oviduct/Uterine Tube/Fallopian tube, Parathyroid Gland, Parotid Salivary glands, Peripheral Blood, Respiratory Bronchiole, Retina, Right Cerebellum, Skin, Synovium/Synovial membrane, Temporal Lobe, Thymus, Tonsils, Umbilical Vein, Urinary Bladder, Vein, Vulva, Whole Organism.

This information was derived by determining the tissue sources of the sequences that were included in the invention including but not limited to SeqCalling sources, Public EST sources, Literature sources, and/or RACE sources.

In addition, the sequence is predicted to be expressed in plasma cells (myeloma) because of the expression pattern of (GENBANK-ID: gb:GENBANK-ID:HSU06631|acc: U06631.1) a closely related Human (H326) mRNA, complete cds homolog.

NOV52 also has homology to the amino acid sequences shown in the BLASTP data listed in Table 52C

TABLE 52C
BLAST results for NOV52

| Gene Index/ | | Length | Identity | Positives | |
|---|---|---|---|---|---|
| Identifier | Protein/Organism | (aa) | (%) | (%) | Expect |
| gi\|13636682\|ref\|XP_{—} | similar to H326 | 597 | 597/597 | 597/597 | 0.0 |
| 010501.2\| | (H. sapiens) | | (100%) | (100%) | |
| (XM_010501) | [Homo sapiens] | | | | |
| gi\|7657148\|ref\|NP_056541.1\| | H326 [Homo | 597 | 588/597 | 589/597 | 0.0 |
| (NM_015726) | sapiens] | | (98%) | (98%) | |
| gi\|17485807\|ref\|XP_{—} | similar to H326 | 779 | 401/603 | 463/603 | 0.0 |
| 066683.1\| | (H. sapiens) | | (66%) | (76%) | |
| (XM_066683) | [Homo sapiens] | | | | |
| gi\|17485821\|ref\|XP_{—} | similar to H326 | 577 | 387/601 | 449/601 | 0.0 |
| 066690.1\| | (H. sapiens) | | (64%) | (74%) | |
| (XM_066690) | [Homo sapiens] | | | | |
| gi\|6679281\|ref\|NP_032847.1\| | plasmacytoma | 747 | 308/469 | 383/469 | 0.0 |
| (NM_008821) | expressed | | (65%) | (80%) | |
| | transcript 2 [Mus | | | | |
| | musculus] | | | | |

The homology between these and other sequences is shown graphically in the ClustalW analysis shown in Table 52D. In the ClustalW alignment of the NOV52 protein, as well as all other ClustalW analyses herein, the black outlined amino acid residues indicate regions of conserved sequence (i.e., regions that may be required to preserve structural or functional properties), whereas non-highlighted amino acid residues are less conserved and can potentially be altered to a much broader extent without altering protein structure or function.

[image]

[image]

[image]

Tables 52E-F list the domain descriptions from DOMAIN analysis results against NOV52. This indicates that the NOV52 sequence has properties similar to those of other proteins known to contain this domain.

TABLE 52E
Domain Analysis of NOV52

gnl|Smart|smart00320, WD40, WD40 repeats; Note
that these repeats are permuted with respect to
the structural repeats (blades) of the beta
propeller domain. (SEQ ID NO:850).
CD-Length = 40 residues, 82.5% aligned
Score = 43.1 bits (100), Expect = 4e−05

| | | | |
|---|---|---|---|
| NOV52: | 189 | LEGHTGCVNTLHFNQRGTWLASGSDDLKVVVWD | 221 |
| | | \|+\|\|\|\| \| ++ \|+ \| \|\|\|\|\|\|\| + +\|\| | |
| Sbjct: | 8 | LKGHTGPVTSVAFSPDGKLLASGSDDGTIKLWD | 40 |

TABLE 52F
Domain Analysis of NOV52

gnl|Pfam|pfam00400, WD40, WD domain, G-beta repeat.
(SEQ ID NO:851)
CD-Length = 39 residues, 97.4% aligned
Score = 43.1 bits (100), Expect= 4e−05

| | | | |
|---|---|---|---|
| NOV52: | 184 | RLQHGLEGHTGCVNTLHFNQRGTWLASGSDDLKVVVWD | 221 |
| | | +\| \| \|\|\|\| \| ++ \|+ \| \|\|\|\|\|\|\| \| +\|\| | |
| Sbjct: | 2 | KLLRTLSGHTGSVTSVAFSPDGNLLASGSDDGTVKIWD | 39 |

Beta-transducin (0-beta) is one of the three subunits (alpha, beta, and gamma) of the guanine nucleotide-binding proteins (G proteins) which act as intermediaries in the transduction of signals generated by transmembrane receptors. The alpha subunit binds to and hydrolyzes GTP; the functions of the beta and gamma subunits are less clear but they seem to be required for the replacement of GDP by GTP as well as for membrane anchoring and receptor recognition.

In higher eukaryotes G-beta exists as a small multigene family of highly conserved proteins of about 340 amino acid residues. Structurally G-beta consists of eight tandem repeats of about 40 residues, each containing a central Trp-Asp motif (this type of repeat is also called a WD-40 repeat). Such a repetitive segment has been shown to exist in a number of other proteins, including G-beta-like peptides, yeast STE4, MSI1, CDC4, CDC20, MAK11, PRP4, PWP1 and TUP1, slime-mould AAC3 and coronin, and Drosophila Groucho protein. The number of repeats within these proteins varies between 5 (PRP4, TUP1, and Groucho) and 8 (G-beta, STE4, MSI1, AAC3, CDC4, PWP1, etc.). In G-beta and G-beta like proteins, the repeats span the entire length of the sequence, while in other proteins, they make up the N-terminal, the central or the C-terminal section.

The protein of this invention contains 7 WD-40 repeats. Although the function of this H326-like protein is not precisely known, it has potential importance in the intracellular transduction of signals, similarly to other WD-40 repeat-containing proteins.

The disclosed NOV52 nucleic acid of the invention encoding a H326-like protein includes the nucleic acid whose sequence is provided in Table 52A or a fragment thereof. The invention also includes a mutant or variant nucleic acid any of whose bases may be changed from the corresponding base shown in Table 52A while still encoding a protein that maintains its H326-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 1 percent of the bases may be so changed.

The disclosed NOV52 protein of the invention includes the H326-like protein whose sequence is provided in Table 52B. The invention also includes a mutant or variant protein any of whose residues may be changed from the corresponding residue shown in Table 52B while still encoding a protein that maintains its H326-like activities and physiological functions, or a functional fragment thereof. In the mutant or variant protein, up to about 36 percent of the residues may be so changed.

The invention further encompasses antibodies and antibody fragments, such as F_{ab} or (F_{ab})₂, that bind immunospecifically to any of the proteins of the invention.

The above disclosed information suggests that this H326-like protein (NOV52) is a member of a “H326 family”. Therefore, the NOV52 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV52 nucleic acids and proteins of the invention are useful in potential therapeutic applications implicated in intracellular transduction of signals and any relevant diseases that may result from dysregulation of signal transduction, and/or other diseases and pathologies.

NOV52 nucleic acids and polypeptides are further useful in the generation of antibodies that bind immuno-specifically to the novel NOV52 substances for use in therapeutic or diagnostic methods. These antibodies may be generated according to methods known in the art, using prediction from hydrophobicity charts, as described in the “Anti-NOVX Antibodies” section below. The disclosed NOV52 protein has multiple hydrophilic regions, each of which can be used as an immunogen. These novel proteins can be used in assay systems for functional analysis of various human disorders, which will help in understanding of pathology of the disease and development of new drug targets for various disorders.

NOV53

A disclosed NOV53 nucleic acid of 1233 nucleotides (also referred to as CG56745-01) encoding a uracil phosphoribosyltransferase-like protein is shown in Table 53A. An open reading frame was identified beginning with a ATG initiation codon at nucleotides 142-144 and ending with a TAA codon at nucleotides 1069-1071. The start and stop codons are shown in bold in Table 53A, and the 5′ and 3′ untranslated regions, if any, are underlined.

TABLE 53A
NOV53 nucleotide sequence.

(SEQ ID NO:195)

CTAGGGGTGAAAGGACAGCCAGGGTTAGATGTTCTGAGGAGGCGGGAGCAACCGAGAGAGCACGTGAGCATC
TGTCCTTTCTACCCGTTCCTCTTTATCTTTAGTGTTCAGTAGCAGCGGGGATAGCCCGGGGCCCGGTGTATG
GCCACGGAGTTACAGTGTCCGGACTCCATGCCCTGTCACAACCAGCAAGTAAACTCTGCCTCAACCCCAAGT
CCCGAGCAGCTGCGACCTGGCGATCTGATCCTGGACCACGCAGGGGGAAACAGAGCCTCCAGGGCCAAGGTG
ATTCTCCTCACGGGGTACGCCCATTCTAGCCTGCCGGCCGAGCTGGACTCTGGGGCCTGCGGCGGCTCCAGC
CTCAACTCAGAGGGCAACAGTGGTAGTGGTGACAGTAGCAGCTATGACGCACCAGCTGGCAACTCCTTCCTA
GAGGACTGCGAACTCTCCCGGCAGATCGGGGCGCAGCTTAAGCTGCTGCCTATGAATGATCAGATACGGGAG
CTACAGACCATCATCCGGGACAAGACAGCCAGTAGAGGTGACTTCATGTTTTCTGCGGATCGTTTGATCAGA
CTTGTTGTGGAAGAGGGATTGAATCAGCTGCCATATAAAGAATGCATGGTGACCACTCCAACAGGGTACAAG
TATGAAGGAGTGAAATTTGAGAAGGGAAATTGTGGGGTCAGCATAATGAGAAGCGGTGAGGCAATGGAACAA
GGTTTACGAGACTGCTGTCGATCCATACGAATTGGAAAGATCCTGATTCAGAGTGATGAGGAGACACAAAGA
GCCAAAGTATATTATGCCAAATTCCCCCCAGACATTTACCGGAGAAAAGTCCTTCTGATGTATCCAATTCTC
AGCACTGGAAATACTGTAATTGAAGCTGTAAAGGTTCTTATAGAACATGGAGTTCAACCCAGTGTTATCATC
CTACTCAGTCTGTTCTCCACTCCTCATGGTGCCAAATCAATCATTCAGGAGTTTCCAGAGATCACAATTTTA
ACTACTGAAGTTCATCCTGTTGCACCTACACATTTTGGACAGAAATACTTTGGAACAGACTAAGTTATTTAA
GTAAAATAATTGTCTTATGTAATATTACAATCATGTTTTGATTTTCTATTTGTTTTACTGATTCACTTGAGG
GTGGCAGAGACAAATGTGTTACAATGCTTTTTAGTTTTGGAAGTGGGTATATTTGAGGTTATATCTCACTTA
GTTATTTGT

In a search of public sequence databases, the NOV53 nucleic acid sequence, located on the X chromosome, has 312 of 544 bases (57%) identical to a gb:GENBANK-ID: YSCFUR1A|acc: M36485.1 mRNA from Saccharomyces cerevisiae (S.cerevisiae uracil phosphoribosyltransferase (FUR1) gene, complete cds).

The disclosed NOV53 polypeptide (SEQ ID NO:196) encoded by SEQ ID NO:195 has 309 amino acid residues and is presented in Table 53B using the one-letter amino acid code. Signal P, Psort and/or Hydropathy results predict that NOV53 has no signal peptide and is likely to be localized to the nucleus with a certainty of 0.3000. Alternatively, NOV53 may also localize to the mitochondrial matrix space with a certainty of 0.1000, or to the lysosome (lumen) with a certainty of 0.1000.

TABLE 53B
Encoded NOV53 protein sequence.

(SEQ ID NO:196)

MATELQCPDSMPCHNQQVNSASTPSPEQLRPGDLILDHAGGNRASRAKVILLTGYAHSSLPAELDSGACGGS
SLNSEGNSGSGDSSSYDAPAGNSFLEDCELSRQIGAQLKLLPMNDQIRELQTIIRDKTASRGDFMFSADRLI
RLVVEEGLNQLPYKECMVTTPTGYKYEGVKFEKGNCGVSIMRSGEAMEQGLRDCCRSIRIGKILIQSDEETQ
RAKVYYAKFPPDIYRRKVLLMYPILSTGNTVIEAVKVLIEHGVQPSVIILLSLFSTPHGAKSIIQEFPEITI
LTTEVHPVAPTHFGQKYFGTD

A search of sequence databases reveals that the NOV53 amino acid sequence has 588 of 597 amino acid residues (98%) identical to, and 138 of 209 amino acid residues (66%) identical to, and 165 of 209 amino acid residues (78%) similar to, the 261 amino acid residue ptnr:SPTREMBL-ACC:Q9VRQ1 protein from Drosophila melanogaster (Fruit fly) (CG5537 Protein) (E=8.5e⁻⁷²).

NOV53 is predicted to be expressed in at least Bone Marrow, Brain, Bladder, Eye, Cervix, Kidney, Liver, Lymph node, Prostate, Small Intestine, Umbilical Vein. This information was derived by determining the tissue sources of the sequences that were included in the invention including but not limited to SeqCalling sources, public EST sources, and/or RACE sources.

NOV53 also has homology to the amino acid sequences shown in the BLASTP data listed in Table 53C

TABLE 53C
BLAST results for NOV53

| Gene Index/ | | Length | Identity | Positives | |
|---|---|---|---|---|---|
| Identifier | Protein/Organism | (aa) | (%) | (%) | Expect |
| gi\|17486179\|ref\|XP_{—} | similar to | 309 | 309/309 | 309/309 | e−172 |
| 060041.1\| | Unknown (protein | | (100%) | (100%) | |
| (XM_060041) | for MGC: 23937) | | | | |
| | (H. sapiens) | | | | |
| | [Homo sapiens] | | | | |
| gi\|14388454\|dbj\|BAB60766.1\| | hypothetical | 309 | 300/309 | 302/309 | e−167 |
| (AB063019) | protein [Macaca | | (97%) | (97%) | |
| | fascicularis] | | | | |
| gi\|13874465\|dbj\|BAB46861.1\| | hypothetical | 309 | 299/309 | 302/309 | e−166 |
| (AB060829) | protein [Macaca | | (96%) | (96%) | |
| | fascicularis] | | | | |
| gi\|14388519\|dbj\|BAB60785.1\| | hypothetical | 309 | 298/309 | 301/309 | e−166 |
| (AB063065) | protein [Macaca | | (96%) | (96%) | |
| | fascicularis] | | | | |
| gi\|8217490\|emb\|CAB92761.1\| | bA311P8.3 | 166 | 166/166 | 166/166 | 3e−94 |
| (AL137013) | (probable uracil | | (100%) | (100%) | |
| | phosphoribosyltranferase) | | | | |
| | [Homo | | | | |
| | sapiens] | | | | |

The homology between these and other sequences is shown graphically in the ClustalW analysis shown in Table 53D. In the ClustalW alignment of the NOV53 protein, as well as all other ClustalW analyses herein, the black outlined amino acid residues indicate regions of conserved sequence (i.e., regions that may be required to preserve structural or functional properties), whereas non-highlighted amino acid residues are less conserved and can potentially be altered to a much broader extent without altering protein structure or function.

[image]

[image]

Table 53E lists the domain descriptions from DOMAIN analysis results against NOV53. This indicates that the NOV53 sequence has properties similar to those of other proteins known to contain this domain.

TABLE 53E
Domain Analysis of NOV53

gnl|Pfam|pfam00156, Pribosyltran, Phosphoribosyl transferase domain.
This family includes a range of diverse phosphoribosyl transferase
enzymes. This family includes: Adenine phosphoribosyltransferase
EC:2.4.2.7, Hypoxanthine-guanine-xanthine phosphoribosyltransferase,
Hypoxanthine phosphoribosyltransferase EC:2.4.2.8, Ribose-phosphate
pyrophosphokinase i EC:2.7.6.1, Amidophosphoribosyltransferase
EC:2.4.2.14, Orotate phosphoribosyltransferase EC:2.4.2.10, Uracil
phosphoribosyltransferase EC:2.4.2.9, Xanthine-guanine
phosphoribosyltransferase EC:2.4.2.22. (SEQ ID NO:852)
CD-Length = 153 residues, 43.1% aligned
Score = 35.0 bits (79), Expect = 0.006

| | | | |
|---|---|---|---|
| NOV53: | 226 | PPDIYRRKVLLMYPILSTGNTVIEAVKVLIEHGVQPSVIILLSLFSTPHGAKSIIQEFPE | 285 |
| | | \|+ ++\|\|++ ++ \|\| \|+ \| ++\| \| \| + + +\| + + + \|\| | |
| Sbjct: | 87 | VGDVGGKRVLIVDDVIDTGGTIRAAAELLKEAGAKVVGVAVLVDRPEGGARERLDKGFPI | 146 |
| NOV53: | 286 | ITILTT 291 | |
| | | +++ | |
| Sbjct: | 147 | PSLIVL 152 | |

The gene of invention is a novel uracil phosphoribosyltransferase (UPRT)-like gene. UPRT catalyzes the formation of uridine 5′-monophosphate in the pyrimidine salvage pathway from uracil and 5-phospho-alpha-D-ribose 1-diphosphate. The Saccharomyces cerevisiae FUR1 gene encodes UPRT (Kern et al., Gene 88:149-157(1990)). Mutations in the FUR1 gene have been correlated to resistance to 5-fluorouracil, a common chemotherapeutic agent (Kern et al., Curr Genet May 1991;19(5):333-7).

The novel gene belongs to a family of phosphoribosyl transferases, as evidenced by the presence of a characteristic domain. It is anticipated that this gene plays a role in the pyrimidine salvage pathway and that it influences the growth or growth restriction of various tissues, and especially of tumor cells.

The disclosed NOV53 nucleic acid of the invention encoding a Uracil Phosphoribosyltransferase-like protein includes the nucleic acid whose sequence is provided in Table 53A or a fragment thereof. The invention also includes a mutant or variant nucleic acid any of whose bases may be changed from the corresponding base shown in Table 53A while still encoding a protein that maintains its Uracil Phosphoribosyltransferase-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 43 percent of the bases may be so changed.

The disclosed NOV53 protein of the invention includes the Uracil Phosphoribosyltransferase-like protein whose sequence is provided in Table 53B. The invention also includes a mutant or variant protein any of whose residues may be changed from the corresponding residue shown in Table 53B while still encoding a protein that maintains its Uracil Phosphoribosyltransferase-like activities and physiological functions, or a functional fragment thereof. In the mutant or variant protein, up to about 4 percent of the residues may be so changed.

The invention further encompasses antibodies and antibody fragments, such as F_{ab} or (F_{ab})₂, that bind immunospecifically to any of the proteins of the invention.

The above disclosed information suggests that this Uracil Phosphoribosyltransferase-like protein (NOV53) is a member of a “Uracil Phosphoribosyltransferase family”. Therefore, the NOV53 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV53 nucleic acids and proteins of the invention are useful in potential therapeutic applications implicated Von Hippel-Lindau (VHL) syndrome, Alzheimer's disease, stroke, tuberous sclerosis, hypercalceimia, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, neurodegeneration, hemophilia, hypercoagulation, idiopathic thrombocytopenic purpura, autoimmune disease, allergies, immunodeficiencies, transplantation, graft versus host disease, fertility disorders, anemia, bleeding disorders, scleroderma, cystitis, incontinence, diabetes, renal artery stenosis, interstitial nephritis, glomerulonephritis, polycystic kidney disease, systemic lupus erythematosus, renal tubular acidosis, IgA nephropathy, hypercalceimia, cirrhosis, inflammatory bowel disease, diverticular disease, lymphedema, cancer, trauma, tissue degeneration, bacterial/viral/parasitic infections, and/or other diseases and pathologies.

NOV53 nucleic acids and polypeptides are further useful in the generation of antibodies that bind immuno-specifically to the novel NOV53 substances for use in therapeutic or diagnostic methods. These antibodies may be generated according to methods known in the art, using prediction from hydrophobicity charts, as described in the “Anti-NOVX Antibodies” section below. The disclosed NOV53 protein has multiple hydrophilic regions, each of which can be used as an immunogen. These novel proteins can be used in assay systems for functional analysis of various human disorders, which will help in understanding of pathology of the disease and development of new drug targets for various disorders.

NOV54

NOV54 includes two novel protein phosphatase 2C-like proteins disclosed below. The disclosed sequences have been named NOV54a and NOV54b.

NOV54a

A disclosed NOV54a nucleic acid of 2185 nucleotides (also referred to as CG56773-01) encoding a protein phosphatase 2C-like protein is shown in Table 54A. An open reading frame was identified beginning with a ATG initiation codon at nucleotides 1-3 and ending with a TGA codon at nucleotides 1402-1404. The start and stop codons are shown in bold in Table 54A, and the 5′ and 3′ untranslated regions, if any, are underlined.

TABLE 54A
NOV54a nucleotide sequence.

(SEQ ID NO:197)

ATGTCCGCCGGCTGGTTCCGGCGCCGCTTCCTGCCTGGGGAGCCGCTCCCCCCGCCGCGGCCGCCTGGGCCG
CATGCCAGCCCCGTGCCCTACCGACGGCCCCGCTTCCTTCGCGGCTCCAGCTCCAGCCCCGGGGCGGCCGAC
GCCTCGCGCCGCCCAGACTCCCGGCCCGTGCGCAGCCCCGCACGAGGACGCACGCTACCCTGGAATGCAGGC
TACGCCGAGATTATCAATGCAGAGAAATCTGAATTCAATGAGGATCAAGCCGCCTGTGGGAAGCTGTGCATC
CGGAGATGTGAGTTTGGGGCTGAAGAACACTGGCTGACCCTGTGCCCAGAGGAGTTCCTGACACCCCATTAC
TGCGCACTGTTCGATGGGCACGGCGGTCCTGCAGCAGCCATCTTGGCTGCCAACACCCTGCACTCCTGCTTG
CGCCQGCAGCTGGAGGCCGTGGTGGAAGGCTTGGTGGCCACTCAGCCCCCCATGCACCTCAATGGCCGCTGC
ATCTGCCCCAGTGACCCTCAGTTTGTGGAGGAAAAGGGCATCAGGGCAGAAGACTTGGTGATCGGGGCATTG
GAGAGTGCCTTTCAGGAATGTGATGAGGTGATCGGGCGGGAGCTGGAGGCCTCAGGCCAGATGGGCGGCTGC
ACAGCCCTGGTGGCTGTGTCCCTGCAGGGAAAGCTGTACATGGCCAATGCTGGGGATAGCAGGGCCATCTTG
GTGCGGAGAGATGACATACGGCCACTGAGCTTCGAGTTCACCCCAGAGACTGAGCGGCAGCGGATCCAGCAG
CTGGCCTTTGTCTATCCTGAGCTTCTGGCTGGTGAGTTCACCCGACTGGAGTTCCCTCGGCGGCTGAAGGGG
GATGACTTGGGACAGAAGGTTTTGTTCAGGGATCACCACATGAGTGGCTGGAGCTACAAACGTGTGGAGAAA
TCGGATCTCAAGTACCCACTGATCCATGGACAGGGTAGGCAGGCTCGGTTACTAGGAACACTGGCTGTCTCC
CGGGGCCTGGGAGACCATCAGCTCAGAGTCCTGGACACAAACATCCAGCTCAAGCCCTTCTTGCTCTCTGTG
CCACAGGTGACTGTGCTGGATGTGGACCAGCTCGAGCTACAGGAGGATGATGTGGTTGTCATGGCAACTGAT
GGACTCTGGGATGTACTGTCCAACGAGCAGGTGGCATGGCTGGTGCGGAGCTTCCTCCCTGGGAACCAAGAG
GACCCACACAGCTATCTGCAGGATGGTCTTCACAGGTTCTCAAAGCTGGCCCAGATGCTCATACACAGCACA
CAGGGAAAGGAAGACAGTCTCACAGAGGAAGGGCAGGTGTCCTACGATGACGTCTCTGTGTTCGTGATTCCC
TTGCACAGTCACGGCCAAGAGAGCAGTGACCACTGAGGATTCAGACACTGTATCCCAGAACTGCTCTAGTGC
CCGGGTGTGGTCTGGGCATCCCTCCAGTGTGACCAAGAGCAAATCCTGCCTGCCCTATCCCTAGCCACCGCC
CAGTGCTCTCACTATCCACCTCAACACACATCCATCTCAAGAGGAACATTTATACCAGGCAGTCAGAGCTGG
AAGTGTATGGAGAGCCCAGCCCACCAGGTCCTGCCTTTTGCGGTGATAACCTTCTCTGGCAGAGTGACTTTA
CAACTTAACTAGGAAACCCATGTGAGGCTCCTCAGACAGGATCTTGAACAGCCCAAAGTATCATTCTCAGAT
AGGGGCACCCAAGCTAAGGGTATTAGCCAAAGATGCCAGGATGGGTAGCTAGCCCATGTTTAGATCCAGGTC
TCCAATTCATGGTTATCAGGGCATGTGTTCAACAACCCCCAAAGTCCACGCAGGTGGCTTGTAGAAACCTTT
GGGCAGCCTCATCTCTGCTAAAACAGCCATCTTCAAGACAGCCCCTGAAAAGAGACCAGTTCAGGTCCTGCC
CTGCTGTTCTTTGCTGGAGATGAGGAACAGGTGCTGGGGCTAAAGTTTGGGCTAGAGCACAAGGGACAACAG
GAACTCTTGGAGTTGGCTGGGTGAGAGGGCTCTCCATTTGCTACCTGTAGTAGCCTGCCTCTTAACTGGTTG
CTTCTCCCTAGTTCCACCCCTGCCCTGGTCTGATGCCCCAACACTGCCCTTGCTTTGTTTTCCCTGTCACCT
CCCTATTATTAAATGTTTTCTACAG

In a search of public sequence databases, the NOV54a nucleic acid sequence, located on the p21.1 region of chromosome 3, has 592 of 928 bases (63%) identical to a gb:GENBANK-ID:AK023315|acc:AK023315.1 mRNA from Homo sapiens (cDNA FLJ13253 fis, clone OVARC1000751) (E=4.5e⁻⁴¹).

A disclosed NOV54a polypeptide (SEQ ID NO:198) encoded by SEQ ID NO:197 has 467 amino acid residues and is presented in Table 54B using the one-letter amino acid code. Signal P, Psort and/or Hydropathy results predict that NOV54a has no signal peptide and is likely to be localized to the microbody (peroxisome) with a certainty of 0.3941. Alternatively, NOV54a may also localize to the nucleus with a certainty of 0.3000, to the lysosome (lumen) with a certainty of 0.1558, or to the mitochondrial matrix space with a certainty of 0.1000.

TABLE 54B
Encoded NOV54a protein sequence.

(SEQ ID NO:198)

MSAGWFRRRFLPGEPLPAPRPPGPHASPVPYRRPRFLRGSSSSPGAADASRRPDSRPVRSPARGRTLPWNAG
YAEIINAEKSEFNEDQAACGKLCIRRCEFGAEEEWLTLCPEEFLTGHYWALFDGHGGPAAAILAANTLHSCL
RRQLEAVVEGLVATQPPMHLNGRCICPSDPQFVEEKGIRAEDLVIGALESAFQECDEVIGRELEASGQMGGC
TALVAVSLQGKLYMANAGDSRAILVRRDEIRPLSFEFTPETERQRIQQLAFVYPELLAGEFTRLEFPRRLKG
DDLGQKVLFRDHHMSGWSYKRVEKSDLKYPLIHGQGRQARLLGTLAVSRGLGDHQLRVLDTNIQLKPFLLSV
PQVTVLDVDQLELQEDDVVVMATDGLWDVLSNEQVAWLVRSFLPGNQEDPHSYLQDGLHRFSKLAQMLIHST
QGKEDSLTEEGQVSYDDVSVFVIPLHSQGQESSDH

A search of sequence databases reveals that the NOV54a amino acid sequence has 32 of 77 amino acid residues (41%) identical to, and 48 of 77 amino acid residues (62%) similar to, the 413 amino acid residue ptnr:SPTREMBL-ACC:Q9M3V1 protein from Fagus sylvatica (Beechnut) (Protein Phpsphatase 2C (PP2C) (EC 3.1.3.16)) (E=9.2e⁻¹⁶).

NOV54a is predicted to be expressed in at least bone marrow, lymphoid tissue, tonsils, brain, colon, uterus, endometrium, placenta, mammary gland/breast, prostate, testis, foreskin, heart, kidney, lung, spleen, peripheral blood, pituitary gland, retina, and pooled germ cell tumors. This information was derived by determining the tissue sources of the sequences that were included in the invention including but not limited to SeqCalling sources, Public EST sources, Literature sources, and/or RACE sources.

In addition, the sequence is predicted to be expressed in ovarian carcinoma because of the expression pattern of(GENBANK-ID: gb:GENBANK-ID:AK023315|acc:AK023315.1) a closely related Homo sapiens cDNA FLJ13253 fis, clone OVARC1000751 homolog.

NOV54b

A disclosed NOV54b nucleic acid of 1930 nucleotides (also referred to as CG56773-02) encoding a protein phosphatase 2C-like protein is shown in Table 54C. An open reading frame was identified beginning with a ATG initiation codon at nucleotides 1-3 and ending with a TGA codon at nucleotides 1147-1149. The start and stop codons are shown in bold in Table 54C, and the 5′ and 3′ untranslated regions, if any, are underlined.

TABLE 54C
NOV54b nucleotide sequence.

(SEQ ID NO:199)

ATGTCCGCCGGCTGGTCCGGCGCCGCTTCCTGCCTGGGGAGCCGCTCCCCGCGCCGCGGCCGCCTGGGCCG
CATGCCAGCCCCGTGCCCTACCGACGGCCCCGCTTCCTTCGCGGCTCCAGCTCCAGCCCCGGGGCGGCCGAC
GCCTCGCGCCGCCCAGACTCCCGGCCCGTGCGCAGCCCCGCACGAGGACGCACGCTACCCTGGAATGCAGGC
TACGCCGAGATTATCAATGCAGAGAAATCTGAATTCAATGAGGATCAAGCCGCCTGTGGGAAGCTGTGCATC
CGGAGATGTGAGTTTGGGGCTGAAGAAGAGTGGCTGACCCTGTGCCCAGAGGAGGATGAGGTGATCGGGCGG
GAGCTGGAGGCCTCAGGCCAGATGGGCGGCTGCACAGCCCTGGTGGCTGTGTCCCTGCAGGGAAAGCTGTAC
ATGGCCAATGCTGGGGATAGCAGGGCCATCTTGGTGCGGAGAGATGAGATACGGCCACTGAGCTTCGAGTTC
ACCCCAGAGACTGAGCGGCAGCGGATCCAGCAGCTGGCCTTTGTCTATCCTGAGCTTCTGGCTGGTGAGTTC
ACCCGACTGGAGTTCCCTCGGCGGCTGAAGGGGGATGACTTGGGACAGAAGGTTTTGTTCAGGGATCACCAC
ATGAGTGGCTGGAGCTACAAACGTGTGGAGAAATCGGATCTCAAGTACCCACTGATCCATGGACAGGGTAGG
CAGGCTCGGTTACTAGGAACACTGGCTGTCTCCCGGGGCCTGGGAGACCATCAGCTCAGAGTCCTGGACACA
AACATCCAGCTCAAGCCCTTCTTGCTCTCTGTGCCACAGGTGACTGTGCTGGATGTGGACCAGCTGGAGCTA
CAGGAGGATGATGTGGTTGTCATGGCAACTGATGGACTCTGGGATGTACTGTCCAACGAGCAGGTGGCATGG
CTGGTGCGGAGCTTCCTCCCTGGGAACCAAGAGGACCCACACAGCTATCTGCAGGATGGTCTTCACAGGTTC
TCAAAGCTGGCCCAGATGCTGATACACAGCACACAGGGAAAGGAAGACAGTCTCACAGAGGAAGGGCAGGTG
TCCTACGATGACGTCTCTGTGTTCGTGATTCCCTTGCACAGTCAGGGCCAAGAGAGCAGTGACCACTGAGGA
TTCAGACACTGTATCCCAGAACTGCTCTAGTGCCCGGGTGTGGTCTGGGCATCCCTCCAGTGTGACCAAGAG
CAAATCCTGCCTGCCCTATCCCTAGCCACCGCCCAGTGCTCTCACTATCCACCTCAACACACATCCATCTCA
AGAGGAACATTTATACCAGGCAGTCAGAGCTGGAAGTGTATGGAGAGCCCAGCCCACCAGGTCCTGCCTTTT
GCGGTGATAACCTTCTCTGGCAGAGTGACTTTACAACTTAACTAGGAAACCCATGTGAGGCTCCTCAGACAG
GATCTTGAACAGCCCAAAGTATCATTCTCAGATAGGGGCACCCAAGCTAAGGGTATTAGCCAAAGATGCCAG
GATGGGTAGCTAGCCCATGTTTAGATCCAGGTCTCCAATTCATGGTTATCAGGGCATGTGTTCAACAACCCC
CAAAGTCCACGCAGGTGGCTTGTAGAAACCTTTGGGCAGCCTCATGTCTGCTAAAACAGCCATCTTCAAGAC
AGCCCCTGAAAAGAGACCAGTTCAGGTCCTGCCCTGCTGTTCTTTGCTGGAGATGAGGAACAGGTGCTGGGG
CTAAAGTTTGGGGTAGAGCACAAGGGACAAGAGGAACTCTTGGAGTTGGCTGGGTGAGAGGGCTCTCCATTT
GCTACCTGTAGTAGCCTGCCTCTTAACTGGTTGCTTCTCCCTAGTTCCAGCCCTGCCCTGGTCTGATGCCCC
AACACTGCCCTTGCTTTGTTTTCCCTGTCACCTCCCTATTATTAAATGTTTTCTACAG

In a search of public sequence databases, the NOV54b nucleic acid sequence, located on chromosome 3, has 446 of 660 bases (67%) identical to a gb:GENBANK-ID:BC011803|acc:BC011803.1 mRNA from Homo sapiens (Homo sapiens, Similar to RIKEN cDNA 2310008J22 gene, clone MGC:19531 IMAGE:4336762, mRNA, complete cds) (E=2.0e⁻⁵⁶).

The disclosed NOV54b polypeptide (SEQ ID NO:200) encoded by SEQ ID NO:199 has 382 amino acid residues and is presented in Table 54D using the one-letter amino acid code. Signal P, Psort and/or Hydropathy results predict that NOV54b has no signal peptide and is likely to be localized to the microbody (peroxisome) with a certainty of 0.4037. Alternatively, NOV54b may also localize to the nucleus with a certainty of 0.3000, to the lysosome (lumen) with a certainty of 0.1000, or to the mitochondrial matrix space with a certainty of 0.1000.

TABLE 54D
Encoded NOV54b protein sequence.

(SEQ ID NO:200)

MSAGWFRRRFLPGEPLPAPRPPGPHASPVPYRRPRFLRGSSSSPGAADASRRPDSRPVRSPARGRTLPWNAG
YAEIINAEKSEFNEDQAACGKLCIRRCEFGAEEEWLTLCPEEDEVIGRELEASGQMGGCTALVAVSLQGKLY
MANAGDSRAILVRRDEIRPLSFEFTPETERQRIQQLAFVYPELLAGEFTRLEFPRRLKGDDLGQKVLFRDHH
QEDDVVVMATDGLWDVLSNEQVAWLVRSFLPGNQEDPHSYLQDGLHRFSKLAQMLIHSTQGKEDSLTEEGQV
SYDDVSVFVIPLHSQGQESSDH

A search of sequence databases reveals that the NOV54b amino acid sequence has 231 of 270 amino acid residues (85%) identical to, and 244 of 270 amino acid residues (90%) similar to, the 453 amino acid residue ptnr:SPTREMBL-ACC:Q9CSD6 protein from Mus musculus (Mouse) (2810423O19RIK Protein) (E=1.6e⁻¹⁶⁷).

NOV54b is predicted to be expressed in at least the following tissues: bone marrow, lymphoid tissue, tonsils, brain, colon, uterus, endometrium, placenta, mammary gland/breast, prostate, testis, foreskin, heart, kidney, lung, spleen, peripheral blood, pituitary gland, retina, and pooled germ cell tumors.

NOV54 also has homology to the amino acid sequences shown in the BLASTP data listed in Table 54E

TABLE 54E
BLAST results for NOV54

| Gene Index/ | | Length | Identity | Positives | |
|---|---|---|---|---|---|
| Identifier | Protein/Organism | (aa) | (%) | (%) | Expect |
| gi\|12850332\|dbj\|BAB28679.1\| | Protein | 453 | 400/459 | 423/459 | 0.0 |
| (AK013149) | phosphatase 2C | | (87%) | (92%) | |
| | containing | | | | |
| | protein˜datasource: | | | | |
| | Pfam, source | | | | |
| | key: PF00481, | | | | |
| | evidence: ISS˜putative | | | | |
| | [Mus | | | | |
| | musculus] | | | | |
| gi\|16552416\|dbj\|BAB71302.1\| | unnamed protein | 270 | 251/252 | 251/252 | e−143 |
| (AK056894) | product [Homo | | (99%) | (99%) | |
| | sapiens] | | | | |
| gi\|17462396\|ref\|XP_{—} | similar to | 247 | 247/255 | 247/255 | e−137 |
| 059571.1\| | putative (H. | | (96%) | (96%) | |
| (XM_059571) | sapiens) [Homo | | | | |
| | sapiens] | | | | |
| gi\|12856386\|dbj\|BAB30649.1\| | Protein | 254 | 222/261 | 234/261 | e−121 |
| (AK017245) | phosphatase 2C | | (85%) | (89%) | |
| | containing | | | | |
| | protein˜datasource: | | | | |
| | Pfam, source | | | | |
| | key: PF00481, | | | | |
| | evidence: ISS˜putative | | | | |
| | [Mus | | | | |
| | musculus] | | | | |
| gi\|17455719\|ref\|XP_{—} | KIAA1157 protein | 514 | 213/480 | 288/480 | 6e−98 |
| 051093.2\| | [Homo sapiens] | | (44%) | (59%) | |
| (XM_051093) | | | | | |

The homology between these and other sequences is shown graphically in the ClustalW analysis shown in Table 54F. In the ClustalW alignment of the NOV54 protein, as well as all other ClustalW analyses herein, the black outlined amino acid residues indicate regions of conserved sequence (i.e., regions that may be required to preserve structural or functional properties), whereas non-highlighted amino acid residues are less conserved and can potentially be altered to a much broader extent without altering protein structure or function.

[image]

[image]

Tables 54G-H list the domain descriptions from DOMAIN analysis results against NOV54. This indicates that the NOV54 sequence has properties similar to those of other proteins known to contain this domain.

TABLE 54G
Domain Analysis of NOV54

gnl|Smart|smart003332, PP2Cc, Serine/threonine phosphatases, family 2C,
catalytic domain; The protein architecture and deduced catalytic
mechanism of PP2C phosphatases are similar to the PP1, PP2A, PP2B
family of protein Ser/Thr phosphatases, with which PP2C shares no
sequence similarity. (SEQ ID NO:853)
CD-Length = 260 residues, 73.5% aligned
Score = 114 bits (286), Expect = 9e−27

| | | | |
|---|---|---|---|
| NOV54: | 118 | GHYWALFDGHGGPAAAILAANTLHSCLRRQLEAVVEGLVATQPPMHLNGRCICPSDPQFV | 177 |
| | | \| ++ +\|\|\|\|\|\| \|\| + \| \| +\| + | |
| Sbjct: | 40 | GGFFGVFDGHGGSEAAKFLSKNLPEILAEELIKDKD------------------------ | 75 |
| NOV54: | 178 | EEKGIRAEDLVIGALESAFQECDEVIGRELEASG-QMGGCTALVAVSLQGKLYMANAGDS | 236 |
| | | ++ \| \|\| \|\| \|\| \| \|\|\|+ \| \| \|\|+\|\|+ \|\|\|+\|\| \|\|\| | |
| Sbjct: | 76 | ------EDEDVEDALRKAFLRTDEEILEELESLEDQRSGTTAVVALIRGNKLYVANVGDS | 129 |
| NOV54: | 237 | RAILVRRDEIRPLSFEFTPETERQRIQQLAFVYPELLAGEFTRLEFPRRLKGDDLGQKVL | 296 |
| | | \|\|_\| \| + \|+ + \| \| +\| | |
| Sbjct: | 130 | RAVLCRNGKAVQLTEDHKPSNEDER----------------------------------- | 154 |
| NOV54: | 297 | FRDHHMSGWSYKRVEKSDLKYPLIHGQGRQARLLGTLAVSRGLGDHQLRVLDTNIQLKPF | 356 |
| | | +\|+ ++ \| \|+ \| \|\|+\|\| \|\|\| \| \|\|+ | |
| Sbjct: | 155 | -----------ERIREA-------GGFVSNGRVNGVLALSRALGDFFL---------KPY | 187 |
| NOV54: | 357 | LLSVPQVTVLDVDQLELQEDDVVVMATDGLWDVLSNEQVAWLVRSFL 403 | |
| | | +++ \| \|\|\|+++ + + +\|+\|\|\|\|\|\|\|\|++\| +\|\| \| | |
| Sbjct: | 188 | VIAEPDVTVVELTEKDDFLI----LASDGLWDVLSNQEVVDIVRKHL 230 | |

TABLE 54H
Domain Analysis of NOV54a

gnl|Pfam|pfam00481, PP2C, Protein phosphatase 2C. Protein phosphatase
2C is a Mn++ or Mg++ dependent protein S/threonine phosphatase.
(SEQ ID NO:854)
CD-Length = 252 residues, 77.0% aligned
Score = 93.2 bits (230), Expect = 3e−20

| | | | |
|---|---|---|---|
| NOV54: | 119 | HYWALFDGHGGPAAAILAANTLHSCLRRQLEAVVEGLVATQPPMHLNGRCICPSDPQFVE | 178 |
| | | ++\|+\|\|\|\|\|\| \|\| \| \| + \| + \| \|+ | |
| Sbjct: | 35 | GFFAVFDGHGGSQAAKYAGKHLETKLALR-----------------------KSFPEL-- | 69 |
| NOV54: | 179 | EKGIRAEDLVIGALESAFQEC-DEVIGRELEASGQMGGCTALVAVSLQGKLYMANAGDSR | 237 |
| | | \| + \|\|+ +\| \| \|\| + + \| \|\|+\|\|+ \|\|\|+\|\| \|\|\|\| | |
| Sbjct: 70 | -------DDLENALKESFLESTDEELRSSAANTDLDSGSTAVVALIRGNKLYVANVGDSR | 122 | |
| NOV54: | 238 | AILVRRDE-IRPLSFEFTP--ETERQRIQQLAFVYPELLAGEFTRLEFPRRLKGDDLGQK | 294 |
| | | \|+\| \| \|+ \|+ + \| \| \|\|+\|\|+ | |
| Sbjct: | 123 | AVLCRNGNAIKQLTEDHKPSNEDERRRIEGAG---------------------------- | 154 |
| NOV54: | 295 | VLFRDHHMSGWSYKRVEKSDLKYPLIHGQGRQARLLGTLAVSRGLGDHQLRVLDTNIQLK | 354 |
| | | + \|\| + \| \|\|\|\|\| \|\| +\|+ + | |
| Sbjct: | 155 | -----------------------GFVSRNGR---VNGVLAVSRAFGDFELK-----PGVL | 183 |
| NOV54: | 355 | PFLLSVPQVTVLDVDQLELQEDDVVVMATDGLWDVLSNEQVAWLVRSFL 403 | |
| | | + + \| \|\| \|+ +++\|+\|\|\|\|\|\|\|\|+++\| +\|\|\| \| | |
| Sbjct: | 184 | QPVTAEPDVT----SHKITPSDEFLILASDGLWDVLSDQEVVDIVRSEL 228 | |

Protein phosphorylation plays a key role in the regulation of cellular functions through the activation or inhibition of enzymes involved in various biochemical pathways. Kinases and phosphatases that determine the phosphorylation state of an enzyme (and its activity) are frequently regulated through the action of hormones and growth factors (1). Four distinct subfamilies of serine/threonine protein phosphatases have been identified in mammals: PP1, PP2A, PP2B and PP2C (2). The PP2C subfamily contains structurally diverse protein phosphatases with a wide range of functions in cellular signal transduction; however, the exact physiological role of most PP2C enzymes is still unclear.

The protein described in this invention contains protein phosphatase 2C domains and is therefore likely to play a role in signal transduction and cellular proliferation. The protein is also homologous, but not identical, to the rat petrin protein, a PP2C subfamily member that has been shown to modulate neurite growth inhibition and may therefore be useful in the treatment of nerve damage resulting from traumatic injury, stroke or CNS degenerative disorders (3). The PP2C-like gene described in this invention is also expressed in the brain and may therefore have similar functions in the CNS. However, it is also expressed in a number of other tissues and based on its expression pattern may contribute to additional human diseases, such as cancer, inflammation/autoimmune diseases, and metabolic disorders. The PP2C-like gene maps to human chromosome 3p21.1.

Protein phosphatase 2C domain is found in protein phosphatase 2C, as well as other proteins e.g. adeylate cyclase. Protein phosphatase 2C (PP2C) is one of the four major classes of mammalian serine/threonine specific protein phosphatases. PP2C is a monomeric enzyme of about 42 Kd that shows broad substrate specificity and is dependent on divalent cations (mainly manganese and magnesium) for its activity. Its exact physiological role is still unclear. Three isozymes are currently known in mammals: PP2C-alpha, -beta and -gamma. In yeast, there are at least four PP2C homologs: phosphatase PTC1 that has weak tyrosine phosphatase activity in addition to its activity on serines, phosphatases PTC2 and PTC3, and hypothetical protein YBR125c. Isozymes of PP2C are also known from Arabidopsis thaliana (ABI1, PPH1), Caenorhabditis elegans (FEM-2, F42G9.1, T23F11.1), Leishmania chagasi and Paramecium tetraurelia. In Arabidopsis thaliana, the kinase associated protein phosphatase (KAPP) is an enzyme that dephosphorylates the Ser/Thr receptor-like kinase RLK5 and which contains a C-terminal PP2C domain.

PP2C does not seem to be evolutionary related to the main family of serine/threonine phosphatases: PP1, PP2A and PP2B. However, it is significantly similar to the catalytic subunit of pyruvate dehydrogenase phosphatase (PDPC), which catalyzes dephosphorylation and concomitant reactivation of the alpha subunit of the E1 component of the pyruvate dehydrogenase complex. PDPC is a mitochondrial enzyme and, like PP2C, is magnesium-dependent.

The disclosed NOV54 nucleic acid of the invention encoding a Protein phosphatase 2C-like protein includes the nucleic acid whose sequence is provided in Table 54A, 54C or a fragment thereof. The invention also includes a mutant or variant nucleic acid any of whose bases may be changed from the corresponding base shown in Table 54A or 54C while still encoding a protein that maintains its Protein phosphatase 2C-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 37 percent of the bases may be so changed.

The disclosed NOV54 protein of the invention includes the Protein phosphatase 2C-like protein whose sequence is provided in Table 54B or 54D. The invention also includes a mutant or variant protein any of whose residues may be changed from the corresponding residue shown in Table 54B or 54D while still encoding a protein that maintains its Protein phosphatase 2C-like activities and physiological functions, or a functional fragment thereof. In the mutant or variant protein, up to about 56 percent of the residues may be so changed.

The invention further encompasses antibodies and antibody fragments, such as F_{ab} or (F_{ab})₂, that bind immunospecifically to any of the proteins of the invention.

The above disclosed information suggests that this Protein phosphatase 2C-like protein (NOV54) is a member of a “Protein phosphatase 2C family”. Therefore, the NOV54 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV54 nucleic acids and proteins of the invention are useful in potential therapeutic applications implicated cancer, trauma, bacterial and viral infections, in vitro and in vivo regeneration, fertility, endometriosis, hypogonadism, cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, transplantation, hemophilia, hypercoagulation, idiopathic thrombocytopenic purpura, autoimmune disease, allergies, immunodeficiencies, transplantation, graft versus host disease (GVHD), lymphaedema, anemia, Alzheimer's disease, stroke, hypercalceimia, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, neurodegeneration, systemic lupus erythematosus, asthma, emphysema, allergy, ARDS, diabetes, renal artery stenosis, interstitial nephritis, glomerulonephritis, polycystic kidney disease, renal tubular acidosis, IgA nephropathy, hypercalceimia, Von Hippel-Lindau (VHL) syndrome, endocrine dysfunctions, growth and reproductive disorders, tonsillitis, Hirschsprung's disease, Crohn's Disease, appendicitis, and/or other diseases and pathologies.

NOV54 nucleic acids and polypeptides are further useful in the generation of antibodies that bind immuno-specifically to the novel NOV54 substances for use in therapeutic or diagnostic methods. These antibodies may be generated according to methods known in the art, using prediction from hydrophobicity charts, as described in the “Anti-NOVX Antibodies” section below. The disclosed NOV54 protein has multiple hydrophilic regions, each of which can be used as an immunogen. These novel proteins can be used in assay systems for functional analysis of various human disorders, which will help in understanding of pathology of the disease and development of new drug targets for various disorders.

NOV55

A disclosed NOV55 nucleic acid of 1500 nucleotides (also referred to as CG56806-01) encoding a Heparan Sulfate 6-Sulfotransferase 3-like protein is shown in Table 55A. An open reading frame was identified beginning with a ATG initiation codon at nucleotides 74-76 and ending with a TGA codon at nucleotides 1490-1492. The start and stop codons are shown in bold in Table 55A, and the 5′ and 3′ untranslated regions, if any, are underlined.

TABLE 55A
NOV55 nucleotide sequence.

(SEQ ID NO:201)

CTTCCGAGCGGGCGCCCGTCCGCCCTGCCGCCGCCGCCGCCGCCGCTTCGCCTGCCGGCCTGAGAGCGGGAC
CATGGATGAAAGGTTCAACAAGTGGCTGCTGACGCCGGTGCTCACTCTCCTCTTCGTGGTCATCATGTACCA
GTACGTGTCCCCCTCCTGCACCAGCTCCTGCACCAACTTCGGGGAGCAGCCCCGCGCGGGGGAGGCCGGCCC
GCCCGCCGTCCCGGGTCCCGCCCGCCGGGCTCAGGCGCCGCCGGAGGAGTGGGAGCAGAGGAGGCCCCAGTT
GCCCCCGCCGCCCCGGGGGCCCCCCGAGGGACCTCGGGGGGCCGCGGCGCCGGAGGAGGAGGACGAGGAGCC
CGGAGACCCCCGGGAGGGGGAGGAAGAGGAGGAGGAAGACGAGCCGGACCCCGAGGCCCCGGAAAACGGCTC
CCTGCCCCGATTCGTGCCGCGCTTCAACTTCAGCCTGAAGGACCTGACCCGCTTCGTGGATTTCAACATCAA
AGGGCGCGACGTGATCGTGTTCCTCCACATCCAGAAGACGGGGGGCACCACTTTCGGCCGGCACCTGGTGAA
GAACATCCGGCTGGAGCAGCCTTGTAGCTGCAAAGCGGGTCAGAAGAAGTGCACCTGCCACCGGCCTGGCAA
GAAGGAGACGTGGCTCTTCTCCCGCTTCTCCACCGGCTGGAGCTGCGGGCTGCACGCCGACTGGACGGAGCT
CACCAACTGCGTGCCGGCCATCATGGAGAAGAAGGACTGTCCCCGCAACCACAGCCACACCAGGAATTTCTA
TTACATCACAATGTTACGGGATCCAGTGTCACGTTACCTGAGCGAGTGGAAACATGTCCAGAGAGGGGCCAC
GGATGACTGGTCTGGGGTCAGCTTGCGGGAGTTTATGGATTGCACCTACAACCTGGCTAACAATCGCCAGGT
GCGCATGCTGGCTGACCTCAGCCTGGTGGGCTGCTATAACTTGACTTTCATGAACGAGAGTGAAAGAAACAC
CATCCTGTTGCAGAGTGCAAAGAACAACCTGAAGAACATGGCCTTCTTTGGGCTCACTGAGTTCCAGAGGAA
GACACAGTTTCTCTTTGAGAGAACATTCAACCTCAAGTTCATCTCCCCCTTCACACAGTTCAACATCACGCG
GGCTTCTAACGTGGAGATCAACGAGGGTGCCCGCCAACGCATTGAGGATCTAAACTTCCTGGACATGCAGCT
TTACGAGTATGCAAAAGATCTCTTCCAGCAGCGCTACCACCACACCAAGCAGCTAGAGCACCAGAGGGACCG
CCAGAAGCGGCGGGAGGAGCGGAGGCTGCAGCGAGAGCACAGGGACCACCAGTGGCCCAAAGAAGATGGGGC
TGCAGAAGGGACTGTCACCGAGGACTACAACAGCCAGGTGGTGAGATGGTGACCTCCTGC

In a search of public sequence databases, the NOV55 nucleic acid sequence, located on chromosome 13, has 1329 of 1492 bases (89%) identical to a gb:GENBANK-ID:AB024567|acc:AB024567.1 mRNA from Mus musculus (mRNA for heparan sulfate 6-sulfotransferase 3, complete cds) (E=7.1e⁻²⁶³).

A disclosed NOV55 polypeptide (SEQ ID NO:202) encoded by SEQ ID NO:201 has 944 amino acid residues and is presented in Table 55B using the one-letter amino acid code. Signal P, Psort and/or Hydropathy results predict that NOV55 has a signal peptide and is likely to be localized to the plasma membrane with a certainty of 0.6850. Alternatively, NOV55 may also localize to the endoplasmic reticulum (membrane) with a certainty of 0.6400, to the Golgi body with a certainty of 0.3700, or to the microbody (peroxisome) with a certainty of 0.1269. The most likely cleavage site for NOV55 is between positions 28 and 29: VSP-SC.

TABLE 55B
Encoded NOV55 protein sequence.

(SEQ ID NO:202)

MDERFNKWLLTPVLTLLFVVIMYQYVSPSCTSSCTNFGEQPRAGEAGPPAVPGPARRAQAPPEEWEQRRPQL
PPPPRGPPEGPRGAAAPEEEDEEPGDPREGEEEEEEDEPDPEAPENGSLPRFVPRFNFSLKDLTRFVDFNIK
GRDVIVFLHIQKTGGTTFGRHLVKNIRLEQPCSCKAGQKKCTCHRPGKKETWLFSRFSTGWSCGLHADWTEL
TNCVPAIMEKKDCPRNHSHTRNFYYITMLRDPVSRYLSEWKHVQRGATWKTSLHMCDGRSPTPDELPTCYPG
DDWSGVSLREFMDCTYNLANNRQVRMLADLSLVGCYNLTFMNESERNTILLQSAKNNLKNMAFFGLTEFQRK
TQFLFERTFNLKFISPFTQFNITRASNVEINEGARQRIEDLNFLDMQLYEYAKDLFQQRYHHTKQLEHQRDR
QKRREERRLQREHRDHQWPKEDGAAEGTVTEDYNSQVVRWMDERFNKWLLTPVLTLLFVVIMYQYVSPSCTS
SCTNFGEQPRAGEAGPPAVPGPARRAQAPPEEWEQRRPQLPPPPRGPPEGPRGAAAPEEEDEEPGDPREGEE
EEEEDEPDPEAPENGSLPRFVPRFNFSLKDLTRFVDFNIKGRDVIVFLHIQKTGGTTFGRHLVKNIRLEQPC
SCKAGQKKCTCHRPGKKETWLFSRFSTGWSCGLHADWTERTNCVPAIMEKKDCPRNHSHTRNFYYITMLRDP
VSRYLSEWKHVQRGATWKTSLHMCDGRSPTPDELPTCYPGDDWSGVSLREFMDCTYNLANNRQVRMLADLSL
VGCYNLTFMNESERNTILLQSAKNNLKNMAFFGLTEFQRKTQFLFERTFNLKFISPFTQFNITRASNVEINE
GARQRIEDLNFLDMQLYEYAKDLFQQRYHHTKQLEHQRDRQKRREERRLQREHRDHQWPKEDGAAEGTVTED
YNSQVVRW

A search of sequence databases reveals that the NOV55 amino acid sequence has 447 of 472 amino acid residues (94%) identical to, and 458 of 472 amino acid residues (97%) similar to, the 470 amino acid residue ptnr:SPTREMBL-ACC:Q9QYK4 protein from Mus musculus (Mouse) (Heparan Sulfate 6-Sulfotransferase 3) (E=7.5e⁻²⁵⁴).

NOV55 is predicted to be expressed in at least Right Cerebellum, Oviduct/Uterine Tube/Fallopian tube, Amygdala, and Kidney. This information was derived by determining the tissue sources of the sequences that were included in the invention including but not limited to SeqCalling sources, Public EST sources, Literature sources, and/or RACE sources.

NOV55 also has homology to the amino acid sequences shown in the BLASTP data listed in Table 55C

TABLE 55C
BLAST results for NOV55

| Gene Index/ | | Length | Identity | Positives | |
|---|---|---|---|---|---|
| Identifier | Protein/Organism | (aa) | (%) | (%) | Expect |
| gi\|7657192\|ref\|NP_056635.1\| | heparan sulfate | 470 | 440/472 | 452/472 | 0.0 |
| (NM_015820) | 6-O- | | (93%) | (95%) | |
| | sulfotransferase | | | | |
| | 3 [Mus musculus] | | | | |
| gi\|16552186\|dbj\|BAB71260.1\| | unnamed protein | 605 | 241/330 | 284/330 | e−144 |
| (AK056706) | product [Homo | | (73%) | (86%) | |
| | sapiens] | | | | |
| gi\|14042611\|dbj\|BAB55322.1\| | unnamed protein | 459 | 242/330 | 285/330 | e−143 |
| (AK027720) | product [Homo | | (73%) | (86%) | |
| | sapiens] | | | | |
| gi\|7657190\|ref\|NP_056634.1\| | heparan sulfate | 506 | 246/369 | 290/369 | e−140 |
| (NM_015819) | 6-O- | | (66%) | (77%) | |
| | sulfotransferase | | | | |
| | 2 [Mus musculus] | | | | |
| gi\|12545389\|ref\|NP_{—} | heparan sulfate | 401 | 238/353 | 286/353 | e−138 |
| 004798.2\| | 6-O- | | (67%) | (80%) | |
| (NM_004807) | sulfotransferase | | | | |
| | [Homo sapiens] | | | | |

The homology between these and other sequences is shown graphically in the ClustalW analysis shown in Table 55D. In the ClustalW alignment of the NOV55 protein, as well as all other ClustalW analyses herein, the black outlined amino acid residues indicate regions of conserved sequence (i.e., regions that may be required to preserve structural or functional properties), whereas non-highlighted amino acid residues are less conserved and can potentially be altered to a much broader extent without altering protein structure or function.

[image]

[image]

[image]

Heparan-sulfate 6-sulfotransferase (HS6ST) catalyzes the transfer of sulfate from 3′-phosphoadenosine 5′-phosphosulfate to position 6 of the N-sulfoglucosamine residue of heparan sulfate.

The disclosed NOV55 nucleic acid of the invention encoding a Heparan Sulfate 6-Sulfotransferase 3-like protein includes the nucleic acid whose sequence is provided in Table 55A, or a fragment thereof. The invention also includes a mutant or variant nucleic acid any of whose bases may be changed from the corresponding base shown in Table 55A while still encoding a protein that maintains its Heparan Sulfate 6-Sulfotransferase 3-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 11 percent of the bases may be so changed.

The disclosed NOV55 protein of the invention includes the Heparan Sulfate 6-Sulfotransferase 3-like protein whose sequence is provided in Table 55B. The invention also includes a mutant or variant protein any of whose residues may be changed from the corresponding residue shown in Table 55B while still encoding a protein that maintains its Heparan Sulfate 6-Sulfotransferase 3-like activities and physiological functions, or a functional fragment thereof. In the mutant or variant protein, up to about 34 percent of the residues may be so changed.

The invention further encompasses antibodies and antibody fragments, such as F_{ab} or (F_{ab})₂, that bind immunospecifically to any of the proteins of the invention.

The above disclosed information suggests that this Heparan Sulfate 6-Sulfotransferase 3-like protein (NOV55) is a member of a “Heparan Sulfate 6-Sulfotransferase 3 family”. Therefore, the NOV55 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV55 nucleic acids and proteins of the invention are useful in potential therapeutic applications implicated in Diabetes, Autoimmune disease, Renal artery stenosis, Interstitial nephritis, Glomerulonephritis, Polycystic kidney disease, Systemic lupus erythematosus, Renal tubular acidosis, IgA nephropathy, Hypercalceimia, Lesch-Nyhan syndrome, Von Hippel-Lindau (VHL) syndrome , Alzheimer's disease, Stroke, Tuberous sclerosis, hypercalceimia, Parkinson's disease, Huntington's disease, Cerebral palsy, Epilepsy, Lesch-Nyhan syndrome, Multiple sclerosis, Ataxia-telangiectasia,Leukodystrophies, Behavioral disorders, Addiction, Anxiety, Pain, Neuroprotection, and/or other diseases and pathologies.

NOV55 nucleic acids and polypeptides are further useful in the generation of antibodies that bind immuno-specifically to the novel NOV55 substances for use in therapeutic or diagnostic methods. These antibodies may be generated according to methods known in the art, using prediction from hydrophobicity charts, as described in the “Anti-NOVX Antibodies” section below. The disclosed NOV55 protein has multiple hydrophilic regions, each of which can be used as an immunogen. These novel proteins can be used in assay systems for functional analysis of various human disorders, which will help in understanding of pathology of the disease and development of new drug targets for various disorders.

NOV56

NOV56 includes two novel N-Hydroxyarylamine Sulfotransferase-like proteins disclosed below. The disclosed sequences have been named NOV56a and NOV56b.

NOV56a

A disclosed NOV56a nucleic acid of 1223 nucleotides (also referred to as CG56816-01) encoding a N-Hydroxyarylamine Sulfotransferase-like protein is shown in Table 56A. An open reading frame was identified beginning with a ATG initiation codon at nucleotides 2-4 and ending with a TGA codon at nucleotides 974-976. The start and stop codons are shown in bold in Table 56A, and the 5′ and 3′ untranslated regions, if any, are underlined.

TABLE 56A
NOV56a nucleotide sequence.

(SEQ ID NO:203)

TATGGCGAAGATTGAGAAAAACGCTCCCACGATGGAAAAAAAGCCAGAACTGTTTAACATCATGGAAGTAGA
TGGAGTCCCTACGTTGATATTATCAAAAGAATGGTGGGAAAAAGTATGTAATTTCCAAGCCAAGCCTGATGA
TCTTATTCATGCATCCATGTTGTACCTGACTTTGGGTAAGTTGCCAGAAGAAGATCATCAGGCTTGGCTTGG
AAATTACCCAAAGTCAGGTACAACATGGATGCATGAAATTTTAGACATGATTCTAAATGATGGTGATGTGGA
GAAATGCAAAAGAGCCCAGACTCTAGATAGACACGCTTTCCTTGAACTGAAATTTCCCCATAAAGAAAAACC
AGATTTGGAGTTCGTTCTTGAAATGTCCTCACCACAACTGATAAAAACACATCTCCCTTCACATCTGATTCC
ACCATCTATCTGGAAAGAAAACTGCAAGATTGTCTATGTGACCAGAAATCCCAAGGATTGCCTGGTGTCCTA
CTACCACTTTCACAGGATGGCTTCCTTTATGCCTGATCCTCAGAACTTAGAGGAATTTTATGAGAAATTCAT
GTCCGGAAAAGTTGTTGGCGGGTCCTGGTTTGACCATATGAAAGGATGGTGGGCTGCAAAAGACATGCACCG
GATCCTCTACCTCTTCTACGAGGATATTAAAAAAAATCCAAAACATGAGATCCACAAGGTGTTGGAATTCTT
GGAGAAAACTTGGTCAGGTGATGTTATAAQACAAGATTGTCCACCATACCTCTTTGATGTAATGAAGGATAA
TCCCATGGCCAACCATACTGCGGTACCTGCTCACATATTCAATCACTCCATCTCAAAATTTATGAGGAAAGG
GATGCCTGGAGACTGGAAGAACCACTTTACTGTGGCTATGAATGAGAACTTTGATAAGCATTATGAAAAGAA
GATGGCAGGGTCCACACTGAACTTCTGCCTGGAGATCTGAGAGGAACAACAACAAACTAGGTGACAGAGACT
ATGCCAACTATTTCGCCTTTTATTCTGTTGAGCAAGGAACTGTGACTGAATGTGGAGCTTATGAGCTTCAGT
CCATCTCCTATAGTGTGGCTAGTTTGCTATAATATTAAAACATGATTTAAAATATCAACAAACCAGTTACTC
CAGCAAATAAAATAAGAGAATTAGAGACCAAAAAAAAAAAAAAAAAAAAAAAAAAAAAAAAAAAAAAAGGG

In a search of public sequence databases, the NOV56a nucleic acid sequence, located on chromosome 2, has 633 of 921 bases (68%) identical to a gb:GENBANK-ID:AF033653|acc:AF033653.1 mRNA from Mus musculus (phenol sulfotransferase mRNA, complete cds) (E=7.3e⁻²⁷⁰).

A disclosed NOV56a polypeptide (SEQ ID NO:204) encoded by SEQ ID NO:203 has 324 amino acid residues and is presented in Table 56B using the one-letter amino acid code. Signal P, Psort and/or Hydropathy results predict that NOV56a has no signal peptide and is likely to be localized to the microbody (peroxisome) with a certainty of 0.7480. Alternatively, NOV56a may also localize to the mitochondrial membrane space with a certainty of 0.1000, or to the lysosome (lumen) with a certainty of 0.1000.

TABLE 56B
Encoded NOV56a protein sequence.

(SEQ ID NO:204)

MAKIEKNAPTMEKKPELFNIMEVDGVPTLILSKEWWEKVCNFQAKPDDLIHASMLYLTLGKLPEEDHQAWLG
NYPKSGTTWMHEILDMILNDGDVEKCKRAQTLDRHAFLELKFPHKEKPDLEFVLEMSSPQLIKTHLPSHLIP
PSIWKENCKIVYVTRNPKDCLVSYYHFHRMASFMPDPQNLEEFYEKFMSGKVVGGSWFDHMKGWWAAKDMHR
ILYLFYEDIKKNPKHEIHKVLEFLEKTWSGDVINKIVHHTSFDVMKDNPMANHTAVPAHIFNHSISKFMRKG
MPGDWKNHFTVAMNENFDKHYEKKMAGSTLNFCLEI

A search of sequence databases reveals that the NOV56a amino acid sequence has 155 of 254 amino acid residues (61%) identical to, and 196 of 254 amino acid residues (77%) similar to, the 304 amino acid residue ptnr:SWISSPROT-ACC:P50237 protein from Rattus norvegicus (Rat) (N-Hydroxyarylamine Sulfotransferase (EC 2.8.2.-) (HAST-I)) (E=6.7e⁻⁹⁶).

NOV56b

In the present invention, the target sequence identified previously, NOV56a, was subjected to the exon linking process to confirm the sequence. PCR primers were designed by starting at the most upstream sequence available, for the forward primer, and at the most downstream sequence available for the reverse primer. In each case, the sequence was examined, walking inward from the respective termini toward the coding sequence, until a suitable sequence that is either unique or highly selective was encountered, or, in the case of the reverse primer, until the stop codon was reached. Such primers were designed based on in silico predictions for the full length cDNA, part (one or more exons) of the DNA or protein sequence of the target sequence, or by translated homology of the predicted exons to closely related human sequences sequences from other species. These primers were then employed in PCR amplification based on the following pool of human cDNAs: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea, uterus. Usually the resulting amplicons were gel purified, cloned and sequenced to high redundancy. The resulting sequences from all clones were assembled with themselves, with other fragments in CuraGen Corporation's database and with public ESTs. Fragments and ESTs were included as components for an assembly when the extent of their identity with another component of the assembly was at least 95% over 50 bp. In addition, sequence traces were evaluated manually and edited for corrections if appropriate. These procedures provide the sequence reported below, which is designated NOV56b. This is identical to the previously identified sequence (NOV56a).

A disclosed NOV56b nucleic acid of 1167 nucleotides (also referred to as CG56816-02) encoding a N-Hydroxyarylamine Sulfotransferase-like protein is shown in Table 56C. An open reading frame was identified beginning with a ATG initiation codon at nucleotides 33-35 and ending with a TGA codon at nucleotides 918-920. The start and stop codons are shown in bold in Table 56C, and the 5′ and 3′ untranslated regions, if any, are underlined.

TABLE 56C
NOV56b nucleotide sequence.

(SEQ ID NO:205)

TATGGCGAAGATTGAGAAAAACGCTCCCACGCATGGGAAAAGAAAGCCAGGAACTGTTTAACATCATGGAAG
TAGATGGAGTCCCTACGTTGATATTATCAAAAGAATGGTGGGAAAAAGTATGTAATTTCCAAGCCAAGCCTG
ATGATCTTATTCTGGCAACTTACCCAAAGTCAGGTACAACATGGATGCATGAAATTTTAGACATGATTCTAA
ATGATGGTGATGTGGAGAAATGCAAAAGAGCCCAGACTCTAGATAGACACGCTTTCCTTGAACTGAAATTTC
CCCATAAAGAAAAACCAGATTTGGAGTTCGTTCTTGAAATGTCCTCACCACAACTGATAAAAACACATCTCC
CTTCACATCTGATTCCACCATCTATCTGGAAAGAAAACTGCAAGATTGTCTATGTGGCCAGAAATCCCAAGG
ATTGCCTGGTGTCCTACTACCACTTTCACAGGATGGCTTCCTTTATGCCTGATCCTCAGAACTTAGAGGAAT
TTTATGAGAAATTCATGTCCGGAAAAGTTGTTGGCGGGTCCTGGTTTGACCATATGAAAGGATGGTGGGCTG
CAAAAGACATGCACCGGATCCTCTACCTCTTCTACGAGGATATTAAAAAAAATCCAAAACATGAGATCCACA
AGGTGTTGGAATTCTTGGAGAAAACTTGGTCAGGTGATGTTATAAACAAGATTGTCCACCATACCTCATTTG
ATGTAATGAAGGATAATCCCATGGCCAACCATACTGCGGTACCTGCTCACATATTCAATCACTCCATCTCAA
AATTTATGAGGAAAGGGATGCCTGGAGACTGGAAGAACCACTTTACTGTGGCTATGAATGAGAACTTTGATA
AGCATTATGAAAAGAAGATGGCAGGGTCCACACTGAACTTCTGCCTGGAGATCTGAGAGGAACAACAACAAA
CTAGGTGACAGAGACTATGCCAACTATTTCGCCTTTTATTCTGTTGAGCAAGGAACTGTGACTGAATGTGGA
GCTTATGAGCTTCAGTCCATCTCCTATAGTGTGGCTAGTTTGCTATAATATTAAAACATGATTTAAAATATC
AACAAACCAGTTACTCCAGCAAATAAAATAAGAGAATTAGAGACCAAAAAAAAAAAAAAAAAAAAAAAAAAA
AAAAAAAAAAAAGGG

In a search of public sequence databases, the NOV56b nucleic acid sequence, located on chromosome 2, has 649 of 919 bases (70%) identical to a gb:GENBANK-ID:AF033653|acc:AF033653.1 mRNA from Mus musculus (phenol sulfotransferase mRNA, complete cds) (E=3.6e⁻⁸⁹).

The disclosed NOV56b polypeptide (SEQ ID NO:206) encoded by SEQ ID NO:205 has 295 amino acid residues and is presented in Table 56D using the one-letter amino acid code. Signal P, Psort and/or Hydropathy results predict that NOV56b has no signal peptide and is likely to be localized to the microbody (peroxisome) with a certainty of 0.7480. Alternatively, NOV56b may also localize to the mitochondrial membrane space with a certainty of 0.1000, or to the lysosome (lumen) with a certainty of 0.1000.

TABLE 56D
Encoded NOV56b protein seqnence.

(SEQ ID NO:206)

MGKESQELFNIMEVDGVPTLILSKEWWEKVCNFQAKPDDLILATYPKSGTTWMHEILDMILNDGDVEKCKRA
QTLDRHAFLELKFPHKEKPDLEFVLEMSSPQLIKTHLPSHLIPPSIWKENCKIVYVARNPKDCLVSYYHFHR
MASFMPDPQNLEEFYEKFMSGKVVGGSWFDHMKGWWAAKDMHRILYLFYEDIKKNPKHEIHKVLEFLEKTWS
GDVINKIVHHTSFDVMKDNPMANHTAVPAHIFNHSISKFMRKGMPGDWKNHFTVAMNENFDKHYEKKMAGST
LNFCLEI

A search of sequence databases reveals that the NOV56b amino acid sequence has 173 of 283 amino acid residues (61%) identical to, and 220 of 283 amino acid residues (77%) similar to, the 304 amino acid residue ptnr:SWISSPROT-ACC:P50237 protein from Rattus norvegicus (Rat) (N-Hydroxyarylamine Sulfotransferase (EC 2.8.2.-) (HAST-I)) (E=4.0e⁻⁹⁹).

NOV56b is predicted to be expressed in at least brain.

NOV56a also has homology to the amino acid sequences shown in the BLASTP data listed in Table 56E

TABLE 56E
BLAST results for NOV56a

| Gene Index/ | | Length | Identity | Positives | |
|---|---|---|---|---|---|
| Identifier | Protein/Organism | (aa) | (%) | (%) | Expect |
| gi\|17446341\|ref\|XP_{—} | similar to | 304 | 299/324 | 301/324 | e−166 |
| 065757.1\| | sulfotransferase, | | (92%) | (92%) | |
| (XM_065757) | phenol preferring | | | | |
| | 2; Phenol | | | | |
| | sulfotransferase | | | | |
| | 1c1 (H. sapiens) | | | | |
| | [Homo sapiens] | | | | |
| gi\|13929030\|ref\|NP_{—} | sulfotransferase, | 304 | 171/303 | 218/303 | 1e−94 |
| 113920.1\| | phenol preferring | | (56%) | (71%) | |
| (NM_031732) | 2; | | | | |
| | Phenolsulfotransferase | | | | |
| | 1c1 [Rattus | | | | |
| | norvegicus] | | | | |
| gi\|9055354\|ref\|NP_061221.1\| | sulfotransferase, | 304 | 172/303 | 217/303 | 4e−94 |
| (NM_018751) | phenol preferring | | (56%) | (70%) | |
| | 2 [Mus musculus] | | | | |
| gi\|16304836\|emb\|CAC95180.1\| | sulfotransferase | 307 | 161/306 | 214/306 | 4e−88 |
| (AJ416889) | 1C [Gallus | | (52%) | (69%) | |
| | gallus] | | | | |
| gi\|14731177\|ref\|XP_{—} | SULT1C | 302 | 161/285 | 201/285 | 2e−85 |
| 010849.3\| | sulfotransferase | | (56%) | (70%) | |
| (XM_010849) | [Homo sapiens] | | | | |

The homology between these and other sequences is shown graphically in the ClustalW analysis shown in Table 56F. In the ClustalW alignment of the NOV56 protein, as well as all other ClustalW analyses herein, the black outlined amino acid residues indicate regions of conserved sequence (i.e., regions that may be required to preserve structural or functional properties), whereas non-highlighted amino acid residues are less conserved and can potentially be altered to a much broader extent without altering protein structure or function.

[image]

[image]

Tables 56G lists the domain descriptions from DOMAIN analysis results against NOV56. This indicates that the NOV56 sequence has properties similar to those of other proteins known to contain this domain.

TABLE 56G
Domain Analysis of NOV56a

gnl|Pfam|pfam00685, Sulfotransfer, Sulfotransferase protein (SEQ ID NO: 855)
CD-Length = 269 residues, 99.6% aligned
Score = 260 bits (665), Expect = 7e−71

| | | | |
|---|---|---|---|
| NOV56: | 25 | GVPTLILSKEWWEKVCN-FQAKPDDLIHASMLYLTLGKLPEEDHQAWLGNYPKSGTTWMH | 83 |
| | | \| \| \| \|+ \| \|\|\|+\|\|\|+ + \|\|\|\|\|\|\|\|+ | |
| Sbjct: | 2G | GFWVDKFHLEGWRKIKNCFQARPDDV--------------------LIAGYPKSGTTWLQ | 41 |
| NOV56: | 84 | EILDMILNDGDVEKCKRAQTLDRHAFLELKFPHKEKPDLEFVLEMSSPQLIKTHLPSHLI | 143 |
| | | \|\|\| + \| \|\| \| \| \|+ +\|\| \|+ \|\|\|+\|\|\|\|\|\|\| \|+ | |
| Sbjct: | 42 | EILSLHPNVGDFEPSPSDPLLFRNPWLEYPKGEDWYETLKP--MPSSPRLIKTHLPLELL | 99 |
| NOV56: | 144 | PPSIWKENCKIVYVTRNPKDCLVSYYHFHRMASFMPDPQN-LEEFYEKFMSGHVVGGSWF | 202 |
| | | \| \| \|\|+\|\| \|\|\|\|\| \|\|\|\|\|\| \| +\| \|\|\| \| \|++ \|+\|\| +\|\| | |
| Sbjct: | 100 | PKSFLSSKAKIIYVLRNPKDVAVSYYHFSRSHKDLPADPGTFEEFLEAFLNGKVLYGSYF | 159 |
| NOV56: | 203 | DHMKGWWAAKDMHRILYLFYEDIKKNPKHEIHKVLEFLEKTWSGDVINKIVHHTSFDVMK | 262 |
| | | \|\|+ \|\|\| + ++\|+\| \|\|\|+\|++\| \|\| \|+ \|\|\| \| + ++\|++ \|+\|\| +\|\| | |
| Sbjct: | 160 | DHVLGWWELRPEPQVLFLDYEDLKEDPAGEIKKIAEFLGLPLSEEELDKLLDHSSFFLMK | 219 |
| NOV56: | 263 | DNPMANHTAVPAHIFNHSISKFMRKGMPGDWKNHFTVAMNENFDKHYEKK 312 | |
| | | \|\|++\|+ + \| \|\|\|\|\|+ \|\|\|\|\|+\|\| \|\| \|\|\| ++\| | |
| Sbjct: | 220 | LNPLSNYETLCLGKSKGRKSPFMRKGLVGDWKNYFTPEQNEKFDKVIKEK 269 | |

This protein carries out sulfation of phenols and bioactivation of n-hydroxyarylamines. It is responsible for the formation of N-hydroxy-2-acetylaminofluorene, a reactive metabolite which exhibits toxicity by binding to DNA, RNA and protein.

Hepatic sulfation of heterocyclic and non-heterocyclic arylamines was studied to assess enzymes responsible for their metabolisms. Both 2-amino-3-methylimidazo[4,5-f]quinoline (IQ)- and non-IQ-type (beta-carboline) heterocyclic amines were N-sulfated to form their sulfamates in cytosols of rat livers in the presence of 3′-phosphoadenosine-5′-phosphosulfate (PAPS). An arylsulfo-transferase, ST1A1, whose cDNA was isolated from a rat cDNA library, was expressed in COS-1 cells. The expressed enzyme catalyzed N-sulfation of IQ, but not appreciably those of 2-amino-1-methyl-6-phenylimidazo[4,5-b]pyridine (Ph1P), 2-amino-6-methyldipyrido[1,2-a:3′,2′-d]imidazole (Glu-P-I), and 3-amino-1-methyl-5H-pyrido[4,3-b]indole (Trp-P-2). N-Sulfation of heterocyclic amines except IQ was higher in hepatic cytosols of female rats than of male rats. These results suggest the involvement of at least plural forms of sulfotransferase on the N-sulfation. In addition, N-sulfation of IQ was also observed in cytosol of a human liver, suggesting that N-sulfation is one of the metabolic pathways of heterocyclic amines in humans as well as rats. Hepatic sulfotransferase also catalyzes metabolic activation of N-hydroxy derivatives of carcinogenic arylamines. Using anti-HAST (hydroxylarylamine sulfotransferase) antibodies and ST1A1 cDNA as screening probes, several cDNA clones were isolated from the cDNA library. A new member of arylsulfotransferase, ST1C1, whose cDNA shows considerable sequence similarity to ST1A1 cDNA, was found to catalyze O-sulfation of N-hydroxy-2-acetylaminofluorence by the cDNA expression in COS-1 cells. From the close similarity of ontogenic profile and sex-specific expression of ST1C1 and HAST, ST1C1 cDNA was shown to encode a major sulfotransferase (HAST) mediating the metabolic activation of N-hydroxyarylamines in rat livers. In addition, properties of PAPS-dependent N-hydroxyarylamine activation and sulfotransferase in human livers are also discussed.

The disclosed NOV56 nucleic acid of the invention encoding a N-Hydroxyarylamine Sulfotransferase-like protein includes the nucleic acid whose sequence is provided in Table 56A, 56C or a fragment thereof. The invention also includes a mutant or variant nucleic acid any of whose bases may be changed from the corresponding base shown in Table 56A or 56C while still encoding a protein that maintains its N-Hydroxyarylamine Sulfotransferase-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 48 percent of the bases may be so changed.

The disclosed NOV56 protein of the invention includes the N-Hydroxyarylamine Sulfotransferase-like protein whose sequence is provided in Table 56B or 56D. The invention also includes a mutant or variant protein any of whose residues may be changed from the corresponding residue shown in Table 56B or 56D while still encoding a protein that maintains its N-Hydroxyarylamine Sulfotransferase-like activities and physiological functions, or a functional fragment thereof. In the mutant or variant protein, up to about 32 percent of the residues may be so changed.

The invention further encompasses antibodies and antibody fragments, such as F_{ab} or (F_{ab})₂, that bind immunospecifically to any of the proteins of the invention.

The above disclosed information suggests that this N-Hydroxyarylamine Sulfotransferase-like protein (NOV56) is a member of a “N-Hydroxyarylamine Sulfotransferase family”. Therefore, the NOV56 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV56 nucleic acids and proteins of the invention are useful in potential therapeutic applications implicated in metabolic diseases and disorders, and/or other diseases and pathologies.

NOV56 nucleic acids and polypeptides are further useful in the generation of antibodies that bind immuno-specifically to the novel NOV56 substances for use in therapeutic or diagnostic methods. These antibodies may be generated according to methods known in the art, using prediction from hydrophobicity charts, as described in the “Anti-NOVX Antibodies” section below. The disclosed NOV56 protein has multiple hydrophilic regions, each of which can be used as an immunogen. These novel proteins can be used in assay systems for functional analysis of various human disorders, which will help in understanding of pathology of the disease and development of new drug targets for various disorders.

NOV57

A disclosed NOV57 nucleic acid of 953 nucleotides (also referred to as CG56829-01) encoding a Testis Specific Serine Kinase-3-like protein is shown in Table 57A. An open reading frame was identified beginning with a ATG initiation codon at nucleotides 50-52 and ending with a TGA codon at nucleotides 854-856. The start and stop codons are shown in bold in Table 57A, and the 5′ and 3′ untranslated regions, if any, are underlined.

TABLE 57A
NOV57 nucleotide sequence.

(SEQ ID NO:207)

CAGAGGCAGCATGAGCTGAGAGGGTGATAGGAAGGCGGCGCTAGACAGCATGGAGGACTTTCTGCTCTCCAA
TGGGTACCAGCTGGGCAAGACCATTGGGGAAGGGACCTACTCAAAAGTCAAAGAAGCATTTTCCAAAAAACA
CCAAAGAAAAGTGGCAATTAAAGTTATAGACAAGATGGGAACTTCCTCAGAGTTTATCCAGAGATTCCTCCC
TCGGGAGCTCCAAATCGTCCGTACCCTGGACCACAAGAACATCATCCAGGTGTATGAGATGCTGGAGTCTGC
CGACGGGAAAATCTGCCTGGTGATGGAGCTCGCTGAGGGAGGGGATGTCTTTGACTGCGTGCTGAATGGGGG
GCCACTGCCTGAAAGCCGGGCCAAGGCCCTCTTCCGTCAGATGGTTGAGGCCATCCGCTACTGCCATGGCTG
TGGCTTTGCCAAGGTGTTGCCCAAGTCACACCGGGAGCTGAGCCAGACCTTCTGCGGCAGTACAGCCTATGC
TGCCCCCGAGGTGCTGCAGGGCATTCCCCACGATAGCAAAAAAGGTGATGTCTGGAGCATGGGTGTGGTCCT
GTATGTCATGCTCTGTGCCAGCCTACCTTTTGACGACACAGACATCCCCAAGATGCTGTGGCAGCAGCAGAA
GGGGGTGTCCTTCCCCACTCATCTGAGCATCTCGGCCGATTGCCAGGACCTGCTCAAGAGGCTCCTGGAACC
CGATATGATCCTCCGGCCTTCAATTGAAGAAGTTAGTTGGCATCCATGGCTAGCAAGCACTTGATAAAAGCA
ATGGCAAGTGCTCTCCAATAAAGTAGGGGGAGAAAGCAAACCCAAAAACCCGCTTCTAAAATGGTGATATAT
ATTTTACGCTTTAAGTT

In a search of public sequence databases, the NOV57 nucleic acid sequence, located on chromosome 1, has 831 of 912 bases (91%) identical to a gb:GENBANK-ID:AF201734|acc:AF201734.1 mRNA from Mus musculus (testis specific serine kinase-3 (Tssk-3) mRNA, complete cds) (E=2.0e⁻¹⁶⁵).

A disclosed NOV57 polypeptide (SEQ ID NO:208) encoded by SEQ ID NO:207 has 268 amino acid residues and is presented in Table 57B using the one-letter amino acid code. Signal P, Psort and/or Hydropathy results predict that NOV57 has no signal peptide and is likely to be localized to the cytoplasm with a certainty of 0.4500. Alternatively, NOV57 may also localize to the microbody (peroxisome) with a certainty of 0.1821, to the mitochondrial matrix space with a certainty of 0.1000, or to the lysosome (lumen) with a certainty of 0.1000.

TABLE 57B
Encoded NOV57 protein sequence.

(SEQ ID NO:208)

MEDFLLSNGYQLGKTIGEGTYSKVKEAFSKKHQRKVAIKVIDKMGTSSEFIQRFLPRELQIVRTLDHKNIIQ
VYEMLESADGKICKVMELAEGGDVFDCVLNGGPLPESRAKALFRQMVEAIRYCHGCGVAHRDLKCENALLQG
FNLKLTDFGFAKVLPKSHRELSQTFCGSTAYAAPEVLQGIPHDSKKGDVWSMGVVLYVMLCASLPFDDTDIP
KMLWQQQKGVSFPTHLSISADCQDLLKRLLEPDMILRPSIEEVSWHPWLAST

A search of sequence databases reveals that the NOV57 amino acid sequence has 240 of 268 amino acid residues (89%) identical to, and 245 of 268 amino acid residues (91%) similar to, the 266 amino acid residue ptnr:SPTREMBL-ACC:Q9JL98 protein from Mus musculus (Mouse) (Testis Specific Serine Kinase-3) (E=6.2e⁻¹²⁴).

NOV57 is predicted to be expressed in at least lung, testis, B-cell, brain, head and neck. Expression information was derived from the tissue sources of the sequences that were included in the derivation of the sequence of CuraGen Ace. No. CG56829-01. The sequence is predicted to be expressed in testis because of the expression pattern of (GENBANK-ID: gb:GENBANK-ID:AF201734|acc:AF201734.1) a closely related Mus musculus testis specific serine kinase-3 (Tssk-3) mRNA, complete cds homolog.

NOV57 also has homology to the amino acid sequences shown in the BLASTP data listed in Table 57C

TABLE 57C
BLAST results for NOV57

| Gene Index/ | | Length | Identity | Positives | |
|---|---|---|---|---|---|
| Identifier | Protein/Organism | (aa) | (%) | (%) | Expect |
| gi\|16418343\|ref\|NP_{—} | testis-specific | 268 | 265/268 | 265/268 | e−157 |
| 443073.1\| | serine/threonine | | (98%) | (98%) | |
| (NM_052841) | kinase 22C [Homo | | | | |
| | sapiens] | | | | |
| gi\|12860201\|dbj\|BAB31876.1\| | data source: MGD, | 268 | 259/268 | 261/268 | e−153 |
| (AK019840) | source | | (96%) | (96%) | |
| | key: MGI: 1929914, | | | | |
| | evidence: ISS˜putative˜ | | | | |
| | serine/threonine | | | | |
| | kinase 22C | | | | |
| | (spermiogenesis | | | | |
| | associated) [Mus | | | | |
| | musculus] | | | | |
| gi\|10946880\|ref\|NP_{—} | serine/threonine | 266 | 240/273 | 245/273 | e−136 |
| 067454.1\| | kinase 22C | | (87%) | (88%) | |
| (NM_021479) | (spermiogenesisassociated); | | | | |
| | testis | | | | |
| | specific serine | | | | |
| | kinase-3 (Tssk-3) | | | | |
| | [Mus musculus] | | | | |
| gi\|16507245\|ref\|NP_{—} | serine/threonine | 358 | 127/266 | 192/266 | 1e−70 |
| 443732.1\| | kinase 22B | | (47%) | (71%) | |
| (NM_053006) | (spermiogenesisassociated); | | | | |
| | testis | | | | |
| | specific serine | | | | |
| | threonine kinase | | | | |
| | 2 [Homo sapiens] | | | | |
| gi\|14776972\|ref\|XP_{—} | serine/threonine | 358 | 127/266 | 192/266 | 1e−70 |
| 033051.1\| | kinase 22B | | (47%) | (71%) | |
| (XM_033051) | (spermiogenesis | | | | |
| | associated) [Homo | | | | |
| | sapiens] | | | | |

The homology between these and other sequences is shown graphically in the ClustalW analysis shown in Table 57D. In the ClustalW alignment of the NOV57 protein, as well as all other ClustalW analyses herein, the black outlined amino acid residues indicate regions of conserved sequence (i.e., regions that may be required to preserve structural or functional properties), whereas non-highlighted amino acid residues are less conserved and can potentially be altered to a much broader extent without altering protein structure or function.

[image]

[image]

Tables 57E-G lists the domain descriptions from DOMAIN analysis results against NOV57. This indicates that the NOV57 sequence has properties similar to those of other proteins known to contain this domain.

TABLE 57E
Domain Analysis of NOV57

gnl|Smart|smart00220, S_TKc, Serine/Threonine protein kinases,
catalytic domain; Phosphotransferases. Serine or threonine-specific
kinase subfamily. (SEQ ID NO:856)
CD-Length = 256 residues, 100.0% aligned
Score = 258 bits (659), Expect = 3e−70

| | | | |
|---|---|---|---|
| NOV57: | 10 | YQLGKTIGEGTYSKVKEAFSKKHQRKVAIKVIDKMGTSSEFIQRFLPRELQIVRTLDHKN | 69 |
| | | \|+\| + +\|+\| + \|\| \| \|\| + \|\|\|\|\|\| \| + +\| \| \|\|++\|++ \|\|\| \| | |
| Sbjct: | 1 | YELLEVLGKGAFGKVYLARDKKTGKLVAIKVIKKEKLKKKKRERIL-REIKILKKLDHPN | 59 |
| NOV57: | 70 | IIQVYEMLESADGKICLVMELAEGGDVFDCVLNGGPLPESRAKALFRQMVEIARYCHGCG | 129 |
| | | \|+++\|++ \| \| \|+ \|\|\|\| \|\|\|\|+\|\| + \| \| \| \|+ \|\|++ \|+ \| \| \| | |
| Sbjct: | 60 | IVKLYDVFED-DDKLYLVMETCEGGDLFDLLKKRGRLSEDEARFYARQILSALEYLHSQG | 118 |
| NOV57: | 130 | VAHRDLKCENALL-QGFNLKLTDFGFAKVLPKSHRELSQTFCGSTAYAAPEVLQGIPHDS | 188 |
| | | + \|\|\|\|\| \|\| \|\| ++\|\| \|\|\| \|\| \| \| \| \|\| \|+ \| \|\|\|\|\| \| + | |
| Sbjct: | 119 | IIHRDLKPENILLDSDGHVKLADFGLAKQL-DSGGTLLTTFVGTPEYMAPEVLLGKGYG- | 176 |
| NOV57: | 189 | KKGDVWSMGVVLYVMLCASLPFDDTDIPKMLWQQ---QKGVSFPTHLSISADCQDLLKRL | 245 |
| | | \| \|+\|\|+\|\|+\|\| +\| \|\| \| \|+++ \| \|\| + +\|\|+\|+\| | |
| Sbjct: | 177 | KAVDIWSLGVILYELLTGKPPFPGDDQLLALFKKIGKPPPPFPPPEWKISPEAKDLIKKL | 236 |
| NOV57: | 246 | LEPDMILRPSIEEVSWHPWL 265 | |
| | | \| \| \| + \|\| \|\|+ | |
| Sbjct: | 237 | LVKDPEKRLTAEEALEHPFF 256 | |

TABLE 57F
Domain Analysis of NOV57

gnl|Pfam|pfam0069, pkinase, Protein kinase domain (SEQ ID NO:857)
CD-Length = 256 residues, 100.0% aligned
Score = 234 bits (596), Expect = 6e−63

| | | | |
|---|---|---|---|
| NOV57: | 10 | YQLGKTIGEGTYSKVKEAFSKKHQRKVAIKVIDKMGTSSEFIQRFLPRELQIVRTLDHKN | 69 |
| | | \|+\|\|+ +\| \| + \|\| + \| \|\|\|\|++ \| \| + ++ \|\|+\|\|+\| \| \| | |
| Sbjct: | 1 | YELGEKLGSGAFGKVYKGKHKDTGEIVAIKILKKRSLSEK--KKRFLREIQILRRLSHPN | 58 |
| NOV57: | 70 | IIQVYEMLESADGKICLVMELAEGGDVFDCVL-NGGPLPESRAKALFRQMVEAIRYCHGC | 128 |
| | | \|+++ + \| \| + \|\|\|\| \|\|\|\|+\|\| + \|\| \| \| \|\| + \|++ + \| \| | |
| Sbjct: | 59 | IVRLLGVFEE-DDHLYLVMEYMEGGDLFDYLRRNGLLLSEKEAKKIALQILRGLEYLHSR | 117 |
| NOV57: | 129 | GVAHRDLKCENALL-QGFNLKLTDFGFAKVLPKSHRELSQTFCGSTAYAAPEVLQGIPHD | 187 |
| | | \|+ \|\|\|\|\| \|\| \|\| + +\|+ \|\|\| \|+ \| \| \| \|\| \|+ \| \|\|\|\|\|+\| | |
| Sbjct: | 118 | GIVHRDLKPENILLDENGTVKIADFGLARKLESSSYEKLTTFVGTPEYMAPEVLEG-RGY | 176 |
| NOV57: | 188 | SKKGDVWSMGVVLYVMLCASLPFDDTDIPKMLWQQQKGVSFPTHL--SISADCQDLLKRL | 245 |
| | | \| \| \|\|\|\|+\|\|+\|\| +\| \|\|\| \| + \|++ ++ \| + \| + +\|\|+\|+ | |
| Sbjct: | 177 | SSKVDVWSLGVILYELLTGKLPFPGIDPLEELFRIKERPRLRLPLPPNCSEELKDLIKKC | 236 |
| NOV57: | 246 | LEPDMILRPSIEEVSWHPWL 265 | |
| | | \| \| \|\|+ +\|+ \|\|\| | |
| Sbjct: | 237 | LNKDPEKRPTAKEILNHPWF 256 | |

TABLE 57G
Domain Analysis of NOV57

gnl|Smart|smart00219, TyrKc, Tyrosine kinase, catalytic domain;
Phosphotransferases. Tyrosine-specific kinase subfamily (SEQ ID NO: 858)
CD-Length = 258 residues, 97.7% aligned
Score = 115 bits (289), Expect = 2e−27

| | | | |
|---|---|---|---|
| NOV57: | 12 | LGKTIGEGTYSKVKEAF---SKKHQRKVAIKVIDKMGTSSEFIQRFLPRELQIVRTLDHK | 68 |
| | | \|\|\| +\|\|\| + +\| + + +\|\|+\| + \| \| + \|+ \|\| \|\| +++\| \|\|\| | |
| Sbjct: | 3 | LGKKLGEGAFGEVYKGTLKGKGGVEVEVAVKTL-KEDASEQQIEEFL-REARLMRKLDHP | 60 |
| NOV57: | 69 | NIIQVYEMLESADGKICLVMELAEGGDVFDCVLNGGP--LPESRAKALFRQMVEAIRYCH | 126 |
| | | \|\|+++ + + + + +\|\|\| \|\|\|\|+ \| + \| \| \| + \|+ + \| | |
| Sbjct: | 61 | NIVKLLGVC-TEEEPLMIVMEYMEGGDLLDYLRKNRPKELSLSDLLSFALQIARGMEYLE | 119 |
| NOV57: | 127 | GCGVAHRDLKCENALL-QGFNLKLTDFGFAK-VLPKSHRELSQTFCGSTAYAAPEVLQGI | 184 |
| | | \|\|\|\| \| \|+ + +\|+ \|\|\| \|+ + + ++ + \|\|\| \|+ | |
| Sbjct: | 120 | SKNFVHRDLAARNCLVGENKTVKIADFGLARDLYDDDYYRKKKSPRLPIRWMAPESLKDG | 179 |
| NOV57: | 185 | PHDSKKGDVWSMGVVLYVML-CASLPFDDTDIPKMLWQQQKGVSFPTHLSISADCQDLLK | 243 |
| | | \| \| \|\|\|\| \|\|+\|+ + \|+ ++\| +\|\| \| + + \|\|+ | |
| Sbjct: | 180 | KFTS-KSDVWSFGVLLWEIFTLGESPYPGMSNEEVLEYLKKGYRLPQPPNCPDEIYDLML | 238 |
| NOV57: | 244 | RLLEPDMILRPSIEEV 259 | |
| | | + \| \|\|+ \|+ | |
| Sbjct: | 239 | QCWAEDPEDRPTFSEL 254 | |

Eukaryotic protein kinases (1) are enzymes that belong to a very extensive family of proteins which share a conserved catalytic core common with both serine/threonine and tyrosine protein kinases. Protein phosphorylation is a fundamental process for the regulation of cellular functions. The coordinated action of both protein kinases and phosphatases controls the levels of phosphorylation and, hence, the activity of specific target proteins. One of the predominant roles of protein phosphorylation is in signal transduction, where extracellular signals are amplified and propagated by a cascade of protein phosphorylation and dephosphorylation events. Two of the best characterized signal transduction pathways involve the cAMP-dependent protein kinase and protein kinase C (PKC). Each pathway uses a different second-messenger molecule to activate the protein kinase, which, in turn, phosphorylates specific target molecules. Extensive comparisons of kinase sequences defined a common catalytic domain, ranging from 250 to 300 amino acids. This domain contains key amino acids conserved between kinases and are thought to play an essential role in catalysis. In the N-terminal extremity of the catalytic domain there is a glycine-rich stretch of residues in the vicinity of a lysine residue, which has been shown to be involved in ATP binding. In the central part of the catalytic domain there is a conserved aspartic acid residue which is important for the catalytic activity of the enzyme (2).

Protein kinases and phosphatases regulate cell-cycle progression, transcription, translation, protein sorting and cell adhesion events that are critical to the inflammatory process. Two of the best-characterized immunosuppressants, cyclosporin and rapamycin, are also effective anti-inflammatory drugs. They act directly on protein phosphorylation and, as such, validate the concept that small-molecule modulators of phosphorylation cascades possess anti-inflammatory properties (3).

Some examples of the role of serine/threonine protein kinases that are important in cell proliferation and disease include AKT, PAF1 and PIM1. Dudek et al. (4) demonstrated that AKT is important for the survival of cerebellar neurons. Thus, the ‘orphan’ kinase moved center stage as a crucial regulator of life and death decisions emanating from the cell membrane. Holland et al. (5) transferred, in a tissue-specific manner, genes encoding activated forms of Ras and Akt to astrocytes and neural progenitors in mice. These authors found that although neither activated Ras nor Akt alone was sufficient to induce glioblastoma multiforme (GBM) formation, the combination of activated Ras and Akt induced high-grade gliomas with the histologic features of human GBMs. These tumors appeared to arise after gene transfer to neural progenitors, but not after transfer to differentiated astrocytes. Increased activity of Ras is found in many human GBMs and Akt activity is increased in most of these tumors, implying that combined activation of these 2 pathways accurately models the biology of this disease (5).

Another disease that involves yet another serine/threonine kinase is Peutz-Jeghers syndrome (PJS), an autosomal dominant disorder characterized by melanocytic macules of the lips, buccal mucosa, and digits, multiple gastrointestinal hamartomatous polyps, and an increased risk of various neoplasms. Jenne et al. (6) identified and characterized the serine/threonine kinase STK11 and identified mutations in PJS patients. All 5 germline mutations were predicted to disrupt the function of the kinase domain. They concluded that germline mutations in STK11, probably in conjunction with acquired genetic defects of the second allele in somatic cells according to the Knudson model, caused the manifestations of PJS. These authors commented that PJS was the first cancer susceptibility syndrome identified that is due to inactivating mutations in a protein kinase and found mutations in the STK11 gene in 11 of 12 unrelated families with PJS. Ten of the 11 were truncating mutations. All were heterozygous in the germline. Su et al. (7) found that of 53 PJS patients with cancer reported to that time, 6 (11%) were diagnosed with pancreatic adenocarcinoma. Su et al. (7) presented evidence that the STK11 gene plays a role in the development of both sporadic and familial (PJS) pancreatic and biliary cancers. They found that in sporadic cancers, the STK11 gene was somatically mutated in 5% of pancreatic cancers and in at least 6% of biliary cancers examined. In the patient with pancreatic cancer associated with PJS, there was inheritance of a mutated copy of the STK11 gene and somatic loss of the remaining wildtype allele.

The novel human serine/threonine protein kinase of the invention contains a protein kinase domain. Therefore it is anticipated that this novel protein has a role in the regulation of essentially all cellular functions and could be a potentially important target for drugs. Such drugs may have important therapeutic applications, such as treating numerous inflammatory diseases.

The disclosed NOV57 nucleic acid of the invention encoding a Testis Specific Serine Kinase-3-like protein includes the nucleic acid whose sequence is provided in Table 57A or a fragment thereof. The invention also includes a mutant or variant nucleic acid any of whose bases may be changed from the corresponding base shown in Table 57A while still encoding a protein that maintains its Testis Specific Serine Kinase-3-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 9 percent of the bases may be so changed.

The disclosed NOV57 protein of the invention includes the Testis Specific Serine Kinase-3-like protein whose sequence is provided in Table 57B. The invention also includes a mutant or variant protein any of whose residues may be changed from the corresponding residue shown in Table 57B while still encoding a protein that maintains its Testis Specific Serine Kinase-3-like activities and physiological functions, or a functional fragment thereof. In the mutant or variant protein, up to about 53 percent of the residues may be so changed.

The invention further encompasses antibodies and antibody fragments, such as F_{ab} or (F_{ab})₂, that bind immunospecifically to any of the proteins of the invention.

The above disclosed information suggests that this Testis Specific Serine Kinase-3-like protein (NOV57) is a member of a “Testis Specific Serine Kinase-3 family”. Therefore, the NOV57 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV57 nucleic acids and proteins of the invention are useful in potential therapeutic applications implicated in metabolic diseases and disorders, and/or other diseases and pathologies.

NOV57 nucleic acids and polypeptides are further useful in the generation of antibodies that bind immuno-specifically to the novel NOV57 substances for use in therapeutic or diagnostic methods. These antibodies may be generated according to methods known in the art, using prediction from hydrophobicity charts, as described in the “Anti-NOVX Antibodies” section below. The disclosed NOV57 protein has multiple hydrophilic regions, each of which can be used as an immunogen. These novel proteins can be used in assay systems for functional analysis of various human disorders, which will help in understanding of pathology of the disease and development of new drug targets for various disorders.

NOV58

NOV58 includes two Gap Junction Beta-5-like proteins, designated herein as NOV58a and NOV58b. Gap junctions are conduits that allow the direct cell-to-cell passage of small cytoplasmic molecules, including ions, metabolic intermediates, and second messengers, and that thereby mediate intercellular metabolic and electrical communication. Gap junction channels consist of connexin protein subunits, which are encoded by a multigene family.

NOV58a

The disclosed NOV58a (alternatively referred to herein as CG56315-01) includes the 728 nucleotide sequence (SEQ ID NO:209) shown in Table 58A. A NOV58a ORF begins with a Kozak consensus ATG initiation codon at nucleotides 28-30 and ends with a stop codon at nucleotides 697-699. The disclosed NOV58a maps to human chromosome 6.

TABLE 58A
NOV58a Nucleotide Sequence

| | |
|---|---|
| AGTCTTGCCTTCTTTTGAGCCTAAGTCATGAGTTGGATGTTCCTCAGAGATCTCCTGAGT | (SEQ ID NO:209) |
| GGAGTAAATAAATACTCCACTGGGACTGGATGGATTTGGCTGGCTGTCGTGTTTGTCTTC | |
| CGTTTGCTGGTCTACATGGTGGCAGCAGAGCACGTGTGGAAAGATGAGCAGAAAGAGTTT | |
| GAGTGCAACAGTAGACAGCCCGGTTGCAAAAATGTGTGTTTTGATGACTTCTTCCCCATT | |
| TCCCAAGTCAGACTTTGGGCCTTACAACTGATAATGGTCTCCACACCTTCACTTCTGGTG | |
| GTTTTACATGTAGCCTATCATGAGGGTAGAGAGAAAAGGCACAGAAAGAAACTCTATGTC | |
| AGCCCAGGTACAATGGATGGGGGCCTATGGTACGCTTATCTTATCAGCCTCATTGTTAAA | |
| ACTGGTTTTGAAATTGGCTTCCTTGTTTTATTTTATAAGCTATATGATGGCTTTAGTGTT | |
| CCCTACCTTATAAAGTGTGATTTGAAGCCTTGTCCCAACACTGTGGACTGCTTCATCTCC | |
| AAACCCACTGAGAAGACGATCTTCATCCTCTTCTTGGTCATCACCTCATGCTTGTGTATT | |
| GTGTTGAATTTCATTGAACTGAGTTTTTTGGTTCTCAAGTGCTTTATTAAGTGCTGTCTC | |
| CAAAAATATTTAAAAAAACCTCAAGTCCTCAGTGTGTGAGTGCCACAGCCTCAGATATGT | |
| TGAATGTG | |

A NOV58a polypeptide (SEQ ID NO:210) encoded by SEQ ID NO:209 is 223 amino acids in length and is presented using the one-letter amino acid code in Table 58B. The Psort profile for NOV58a predicts that this sequence has a signal peptide and is likely to be localized at the plasma membrane with a certainty of 0.6000. In alternative embodiments, a NOV58a polypeptide is located to the mitochondrial inner membrane with a certainty of 0.4358, to the endoplasmic reticulum (membrane) with a certainty of 0.3000, or to the Golgi with a certainty of 0.4000. The Signal P predicts a likely cleavage site for a NOV58a peptide is between positions 40 and 41, i.e., at the dash in the sequence VAA-EH.

TABLE 58B
NOV58a Polypeptide Sequence

| | |
|---|---|
| MSWMFLRDLLSGVNKYSTGTGWIWLAVVFVFRLLVYMVAAEHVWKDEQKEFECNSRQPGC | (SEQ ID NO:210) |
| KNVCFDDFFPISQVRLWALQLIMVSTPSLLVVLHVAYHEGREKRHRKKLYVSPGTMDGGL | |
| WYAYLISLIVKTGFEIGFLVLFYKLYDGFSVPYLIKCDLKPCPNTVDCFISKPTEKTIFI | |
| LFLVITSCLCIVLNFIELSFLVLKCFIKCCLQKYLKKPQVLSV | |

NOV58b

The disclosed NOV58b (alternatively referred to herein as CG56315-02) includes the 727 nucleotide sequence (SEQ ID NO:211) shown in Table 58C. A SEC2 ORF begins with a Kozak consensus ATG initiation codon at nucleotides 27-29 and ends with a stop codon at nucleotides 696-698. The disclosed NOV58b maps to human chromosome 10.

TABLE 58C
NOV58b Nucleotide Sequence

| | |
|---|---|
| AGTCTTGCTTCTTTTGAGCCTAAGTCATGAGTTGGATGTTCCTCAGAGATCTCCTGAGTG | (SEQ ID NO:211) |
| GAGTAAATAAATACTCCACTGGGATTGGATGGATTTGGCTGGCTGTCGTGTTTGTCTTCC | |
| GTTTGCTGGTCTACATGGTGGCAGCAGAGCACGTGTGGAAAGATGAGCAGAAAGAGTTTG | |
| AGTGCAACAGTAGACAGCCCGGTTGCAAAAATGTGTGTTTTGATGACTTCTTCCCCATTT | |
| CCCAAGTCAGACTTTGGGCCTTACAACTGATAATGGTCTCCACACCTTCACTTCTGGTGG | |
| TTTTACATGTAGCCTATCATGAGGGTAGAGAGAAAAGGCACAGAAAGAAACTCTATGTCA | |
| GCCCAGGTACAATGGATGGGGGCCTATGGTACGCTTATCTTATCAGCCTCATTGTTAAAA | |
| CTGGTTTTGAAATTGGCTTCCTTGTTTTATTTTATAAGCTATATGATGGCTTTAGTGTTC | |
| CCTACCTTATAAAGTGTGATTTGAAGCCTTGTCCCAACACTGTGGACTGCTTCATCTCCA | |
| AACCCACTGAGAAGACGATCTTCATCCTCTTCTTGGTCATCACCTCATGCTTGTGTATTG | |
| TGTTGAATTTCATTGAACTGAGTTTTTTGGTTCTCAAGTGCTTTATTAAGTGCTGTCTCC | |
| AAAAATATTTAAAAAAACCTCAAGTCCTCAGTGTGTGAGTGCCACAGCCTCAGATATGTT | |
| GAATGTG | |

A NOV58b polypeptide (SEQ ID NO:212) encoded by SEQ ID NO:211 is 628 amino acids in length and is presented using the one-letter amino acid code in Table 58D. The Psort profile for NOV58b predicts that this sequence has a signal peptide and is likely to be localized at the plasma membrane with a certainty of 0.6000. The Signal P predicts a likely cleavage site for a NOV58b peptide is between positions 40 and 41, i.e., at the dash in the sequence VAA-EH.

TABLE 58D
NOV58b Polypeptide Sequence

| | |
|---|---|
| MSWMFLRDLLSGVNKYSTGIGWIWLAVVFVFRLLVYMVAAEHVWKDEQKEFECNSRQPGC | (SEQ ID NO:212) |
| KNVDFDDFFPISQVRLWALQLIMVSTPSLLVVLHVAYHEGREKRHRKKLYVSPGTMDGGL | |
| WYAYLISLIVKTGFEIGFLVLFYKLYDGFSVPYLIKCDLKPCPNTVDCFISKPTEKTIFI | |
| LFLVITSCLCIVNFIELSFLVLKCFIKCCLQKYLKKPQVLSV | |

A BLAST analysis of NOV58 was run against the proprietary PatP GENESEQ Protein Patent database. It was found, for example, that the amino acid sequence of NOV58 had high homology to other proteins as shown in Table 58E.

TABLE 58E
BLASTX results from PatP database for NOV58

| | | Smallest |
|---|---|---|
| | | Sum |
| | High | Probability |
| Sequences producing High-scoring Segment Pairs: | Score | P(N) |
| patp:AAY32079 Human gap junction protein beta-4 | 689 | 1.2e−67 |
| patp:AAY36145 Human secreted protein #17 - | 689 | 1.2e−67 |
| Homo sapiens | | |
| patp:AAY36192 Human secreted protein #64 - | 689 | 1.2e−67 |
| Homo sapiens | | |
| patp:AAY70457 Human membrane channel | 666 | 3.3e−65 |
| protein-7 (MECHP-7) | | |
| patp:AAC74001 Human colon cancer antigen | 657 | 3.0e−64 |
| protein | | |

In a search of public sequence databases, it was found, for example, that the nucleic acid sequence of this invention has 240 of 336 bases (71%) identical to a gb:GENBANK-ID:RATCXN311A|acc:M76533.1 mRNA from Rattus norvegicus (Rattus norvegicus connexin (CXN-311) gene). The full amino acid sequence of the protein of the invention was found to have 125 of 220 amino acid residues (56%) identical to, and 170 of 220 amino acid residues (77%) similar to, the 271 amino acid residue ptnr:SWISSNEW-ACC:Q02739 protein from Mus musculus (Mouse) (GAP JUNCTION BETA-5 PROTEIN (CONNEXIN 31.1) (CX31.1)). NOV58 also has homology to the other proteins shown in the BLASTP data in Table 58F.

TABLE 58F
NOV58 BLASTP results

| Gene Index/ | | Length | Identity | Positive | |
|---|---|---|---|---|---|
| Identifier | Protein/Organism | (aa) | (%) | (%) | Expect |
| gi\|15990851\|emb\| | connexin25 | 223 | 222/223 | 222/223 | e−116 |
| CAC93845.1\| | [Homo sapiens] | | (99) | (99) | |
| (AJ414563) | | | | | |
| gi\|10835079\|ref\| | gap junction protein, | 273 | 123/216 | 161/216 | 1e−67 |
| NP_005259.1\| | beta 5 (connexin 31.1) | | (56) | (73) | |
| (NM_005268) | [Homo sapiens] | | | | |
| gi\|6753996\|ref\| | gap junction membrane | 271 | 123/214 | 166/214 | 1e−67 |
| NP_034421.1\| | channel protein beta 5; | | (57) | (77) | |
| (NM_010291) | connexin 31.1 | | | | |
| | [Mus musculus] | | | | |
| gi\|15029850\|gb\| | Similar to gap junction | 271 | 123/214 | 166/214 | 7e−67 |
| AAH11148.1\| | membrane channel protein | | (57) | (77) | |
| AAH11148 | beta 5 | | | | |
| (BC011148) | [Mus musculus] | | | | |
| gi\|4009522\|gb\| | connexin 31.1 | 273 | 122/216 | 160/216 | 7e−67 |
| AAC95472.1\| | [Homo sapiens] | | (56) | (73) | |
| (AF099731) | | | | | |

This BLASTP data is displayed graphically in the ClustalW in Table 58G. A multiple sequence alignment is given, with the NOV58a and b protein being shown on line 1 and 2 in a ClustalW analysis comparing the protein of the invention with the related protein sequences shown in Table 58F.

[image]

[image]

The presence of identifiable domains in NOV58 was determined by searches using software algorithms such as PROSITE, DOMAIN, Blocks, Pfam, ProDomain, and Prints, and then determining the Interpro number by crossing the domain match (or numbers) using the Interpro website (http:www.ebi.ac.uk/interpro). DOMAIN results for NOV58 as disclosed in Table 58H, were collected from the Conserved Domain Database (CDD) with Reverse Position Specific BLAST analyses. This BLAST analysis software samples domains found in the Smart and Pfam collections. For Table 58H fully conserved single residues are indicated by the sign (|) and “strong” semi-conserved residues are indicated by the sign (+). The “strong” group of conserved amino acid residues may be any one of the following groups of amino acids: STA, NEQK, NHQK, NDEQ, QHRK, MILV, MILF, HY, FYW.

Table 58H lists the domain description from DOMAIN analysis results against NOV58. This indicates that the NOV58 sequence has properties similar to those of other proteins known to contain this domain.

TABLE 58H
Domain Analysis of NOV58

gnl|Pfam|pfam00029, connexin, Connexin. SEQ ID NO:859
CD-Length = 218 residues, 100.0% aligned
Score = 265 bits (678), Expect = 2e−72

| | | | |
|---|---|---|---|
| NOV58: | 1 | MSWMFLRDLLSGVNKYSTGTGWIWLAVVFVFRLLVYMVAAEHVWKDEQKEFECNSRQPGC | 60 |
| | | M W FL LL GVNK+ST G IWL+V+F+FR+LV VAAE VW DEQ +F CN++QPGC | |
| Sbjct: | 1 | MDWSFLGRLLEGVNKHSTAIGKIWLSVLFIFRILVLGVAAESVWGDEQSDFVCNTQQPGC | 60 |
| NOV58: | 61 | KNVDFDDFFPISQVRLWALQLIMVSTPSLLVVLHVAYHEGREKRHRKK-------LYVSP | 113 |
| | | +NVC+D FFPIS VRLW LQLI VSTPSLL + HVAY RE++ R+K LY | |
| Sbjct: | 61 | ENVCYDQFFPISHVRLWVLQLIFVSTPSLLYLGHVAYRVRREEKLREKEEEHSKGLYSEE | 120 |
| NOV58: | 114 | G-----------TMDGGLWYAYLISLIVKTGFEIGFLVLFYKLYDGFSVPYLIKCDLKPC | 162 |
| | | + GGLW+ Y+ S+I K+ FE+GFL Y LY GF++ L+ C PC | |
| Sbjct: 121 | AKKRCGSEDGKVRIRGGLWWTYVFSIIFKSIFEVGFLYGQYLLY-GFTMSPLVVCSRAPC | 179 | |
| NOV58: | 163 | PNTVDCFISKPTEKTIFILFLVITSCLCIVLNFIELSFL 201 | |
| | | P+TVDCF+S+PTEKTIFI+F+++ S +C++LN EL +L | |
| Sbjct: | 180 | PHTVDCFVSRPTEKTIFIVFMLVVSAICLLLNLAELFYL 218 | |

Connexins are a family of integral membrane proteins that oligomerise to form intercellular channels that are clustered at gap junctions. These channels are specialized sites of cell-cell contact that allow the passage of ions, intracellular metabolites and messenger molecules (with molecular weight<1-2 kD) from the cytoplasm of one cell to its apposing neighbours. They are found in almost all vertebrate cell types, and somewhat similar proteins have been cloned from plant species. Invertebrates utilise a different family of molecules, innexins, that share a similar predicted secondary structure to the vertebrate connexins, but have no sequence identity to them.

Vertebrate gap junction channels are thought to participate in diverse biological functions. For instance, in the heart they permit the rapid cell-cell transfer of action potentials, ensuring coordinated contraction of the cardiomyocytes. They are also responsible for neurotransmission at specialised ‘electrical’ synapses. In non-excitable tissues, such as the liver, they may allow metabolic cooperation between cells. In the brain, glial cells are extensively-coupled by gap junctions; this allows waves of intracellular Ca2+ to propagate through nervous tissue, and contribute to their ability to spatially-buffer local changes in extracellular K+ concentration.

The connexin protein family is encoded by at least 13 genes in rodents, with many homologues cloned from other species. They show overlapping tissue expression patterns, most tissues expressing more than one connexin type. Their conductances, permeability to different molecules, phosphorylation and voltage-dependence of their gating, have been found to vary. Possible communication diversity is increased further by the fact that gap junctions may be formed by the association of different connexin isoforms from apposing cells. However, in vitro studies have shown that not all possible combinations of connexins produce active channels.

Hydropathy analysis predicts that all cloned connexins share a common transmembrane (TM) topology. Each connexin is thought to contain 4 TM domains, with two extracellular and three cytoplasmic regions. This model has been validated for several of the family members by in vitro biochemical analysis. Both N- and C-termini are thought to face the cytoplasm, and the third TM domain has an amphipathic character, suggesting that it contributes to the lining of the formed-channel. Amino acid sequence identity between the isoforms is ˜50-80%, with the TM domains being well conserved. Both extracellular loops contain characteristically conserved cysteine residues, which likely form intramolecular disulphide bonds. By contrast, the single putative intracellular loop (between TM domains 2 and 3) and the cytoplasmic C-terminus are highly variable among the family members. Six connexins are thought to associate to form a hemi-channel, or connexon. Two connexons then interact (likely via the extracellular loops of their connexins) to form the complete gap junction channel. Two sets of nomenclature have been used to identify the connexins. The first, and most commonly used, classifies the connexin molecules according to molecular weight, such as connexin43 (abbreviated to Cx43), indicating a connexin of molecular weight close to 43 kD. However, studies have revealed cases where clear functional homologues exist across species that have quite different molecular masses; therefore, an alternative nomenclature was proposed based on evolutionary considerations, which divides the family into two major subclasses, alpha and beta, each with a number of members.

Due to their ubiquity and overlapping tissue distributions, it has proved difficult to elucidate the functions of individual connexin isoforms. To circumvent this problem, particular connexin-encoding genes have been subjected to targeted-disruption in mice, and the phenotype of the resulting animals investigated. Around half the connexin isoforms have been investigated in this manner. Further insight into the functional roles of connexins has come from the discovery that a number of human diseases are caused by mutations in connexin genes. For instance, mutations in Cx32 give rise to a form of inherited peripheral neuropathy called X-linked dominant Charcot-Marie-Tooth disease. Similarly, mutations in Cx26 are responsible for both autosomal recessive and dominant forms of nonsyndromic deafness, a disorder characterised by hearing loss, with no apparent effects on other organ systems.

The disclosed NOV58 is a connexin-like protein localized to gap junctions. Gap junctions were first characterized by electron microscopy as regionally specialized structures on plasma membranes of contacting adherent cells. These structures were shown to consist of cell-to-cell channels. Proteins, called connexins, purified from fractions of enriched gap junctions from different tissues differ. The connexins are designated by their molecular mass. Another system of nomenclature divides gap junction proteins into 2 categories, alpha and beta, according to sequence similarities at the nucleotide and amino acid levels. For example, CX43 is designated alpha-1 gap junction protein, whereas CX32 and CX26 are called beta-1 and beta-2 gap junction proteins, respectively. This nomenclature emphasizes that CX32 and CX26 are more homologous to each other than either of them is to CX43.

Willecke et al. (1990) used rat connexin gene probes in Southern blot analysis of human-mouse somatic cell hybrids to map the CX26 gene to chromosome 13. By means of somatic cell hybrids, Hsieh et al. (1991) assigned the GJB2 gene to chromosome 13 in man and chromosome 14 in the mouse. Haefliger et al. (1992) showed that the rat homologs of the CX26 and CX46 genes are tightly linked on chromosome 14. By isotopic in situ hybridization, Mignon et al. (1996) mapped GJB2 to 13q11-q12 and confirmed the assignment to mouse chromosome 14. Kelsell et al. (1997) studied a pedigree containing individuals with autosomal dominant deafness and identified a mutation in the CX26 gene: a 101T-C transition resulting in a met34-to-thr amino acid substitution. CX26 mutations resulting in premature stop codons were also found in 3 autosomal recessive nonsyndromic sensorineural deafness pedigrees, genetically linked to 13q11-q12, where the CX26 gene is localized.

Immunohistochemical staining of human cochlear cells for CX26 demonstrated high levels of expression. Kelley et al. (1998) presented evidence that the 101T-C missense mutation identified by Kelsell et al. (1997) in individuals with autosomal dominant nonsyndromic deafness is not sufficient to cause hearing loss. Carrasquillo et al. (1997) performed linkage analysis in 2 interrelated inbred kindreds in a single Israeli-Arab village containing more than 50 individuals with nonsyndromic recessive deafness. Genetic mapping demonstrated that a gene located at 13q11 (DFNB1) segregated with the deafness in these 2 kindreds. Haplotype analysis, using 8 microsatellite markers spanning 15 cM in 13q11, suggested the segregation of 2 different mutations in this extended kindred; affected individuals were homozygotes for either haplotype or compound heterozygotes. Carrasquillo et al. (1997) identified 2 distinct mutations, trp77 to arg and 35delG, in the CX26 gene, both of which were predicted to inactivate connexin 26.

The recombination of marker alleles involving polymorphisms in 13q11, at known map distances from the mutations, allowed them to estimate the age of the mutations to be 3 to 5 generations (75 to 125 years). The study demonstrated that in small populations with high rates of consanguinity, as compared with large outbred populations, recessive mutations may have very recent origin and show allelic diversity. They pointed to the same phenomenon being observed for Hurler syndrome with 3 unique mutations and for metachromatic leukodystrophy with 5 distinct mutations, discovered among the Druze and Muslim Arab villages in Israel. In light of these findings, the authors commented that it is likely that homozygosity mapping studies in highly inbred communities may be compromised, as may be studies of mapping by linkage disequilibrium, unless the possibility of mutational diversity is taken into account.

Lench et al. (1998) studied the role of CX26 mutations in singleton (sporadic) cases of nonsyndromal sensorineural deafness. Such mutations were identified in 4 of 43 U.K. and 2 of 25 Belgian patients. Thus, about 10% of families presenting with a child sporadically affected with this disorder can be offered definitive mendelian recurrence risks. This was said to be the first genetic test available for screening such children. Kelley et al. (1998) analyzed 58 multiplex families each having at least 2 affected children diagnosed with autosomal recessive nonsyndromic deafness. Mutations in both alleles of GJB2 were observed in 20 of the 58 families. A 30delG allele occurred in 33 of the 116 chromosomes, for a frequency of 0.284. This mutation was observed in 2 of 192 control chromosomes, for an estimated gene frequency of 0.01+/−0.007. The homozygous frequency of the 30delG allele was then estimated at 0.0001, or 1 in 10,000. Given that the frequency of all childhood hearing impairment is 1 in 1,000 and that half of that is genetic, the specific mutation 30delG is responsible for 10% of all childhood hearing loss and for 20% of all childhood hereditary hearing loss. Six novel mutations were also observed in the affected population.

Murgia et al. (1999) studied 53 unrelated individuals with nonsyndromic sensorineural hearing impairment and carried out CX26 mutation analysis. Mutations were found in 53% of cases, in 35.3% of those in whom autosomal recessive inheritance was thought likely and in 60% of the presumed sporadic cases. Three novel mutations were found. The hearing deficit varied from mild to profound even within the same family. Among patients with profound hearing loss, 35.5% were found to have a mutation; among those severely impaired, 20%; and among those moderately impaired, 33.3%.

Rabionet et al. (2000) analyzed the GJB2 gene in 576 families/unrelated patients with recessive or sporadic deafness from Italy and Spain, 193 of them being referred as autosomal recessive and the other 383 as apparently sporadic. Of the 1,152 unrelated GJB2 chromosomes, 37% had GJB2 mutations. A total of 23 different mutations were detected. Mutation 35delG was the most common, accounting for 82% of all GJB2 deafness alleles. It represented 88% of the alleles in Italian patients and only 55% in Spanish cases. Sobe et al. (2000) sequenced the entire coding region of the GJB2 gene in 75 hearing-impaired children and adults in Israel. Was both prelingual and postlingual, with hearing loss ranging from moderate to profound. Almost 39% of all persons tested harbored GJB2 mutations, most of which were 35delG and 167delT. A novel mutation, involving both a deletion and an insertion, 51del12insA, was identified in a family originating from Uzbekistan. All GJB2 mutations were associated with prelingual hearing loss, although severity ranged from moderate to profound, with variability even among hearing-impaired sibs. No significant difference in hearing levels was found between individuals with 35delG and 167delT mutations.

Antoniadi et al. (2000) screened 26 unrelated Greek patients with prelingual sensorineural deafness in whom syndromic forms and environmental causes of deafness had been excluded. They detected the 35delG mutation in 28 chromosomes (53.8%); another 3 sequence variations accounted for 7.6% of the alleles. Wilcox et al. (2000) performed mutation analysis of the GJB2 gene and audiology on 106 families presenting with at least 1 child with congenital hearing loss. In 74 families (80 children), the etiology was consistent with nonsyndromic recessive hearing loss. Six different GJB2 mutations, including 1 novel mutation, were identified. They found that GJB2 mutations caused a range of phenotypes from mild to profound hearing impairment and that loss of hearing in the high-frequency range (4,000 to 8,000 Hz) is a characteristic feature in children with molecularly diagnosed CX26 hearing impairment. They also demonstrated that high frequency hearing loss was found in a group of similar size of deaf children in whom a mutation could be found in only one of the GJB2 alleles. In their study, the M34T mutation was associated with hearing loss only when present in compound heterozygous state, suggesting autosomal recessive inheritance. Heathcote et al. (2000) reported a missense mutation in affected members of a family with autosomal dominant deafness and palmoplantar keratoderma. Rabionet et al. (2000) reviewed the molecular genetics of hearing impairment due to mutations in gap junction genes encoding beta-connexins. Among these genes, mutations in GJB2 account for about 50% of all congenital cases of hearing impairment. Three mutations in GJB2 are particularly common in specific populations: 35delG in Caucasoids, 167delT in Ashkenazi Jews, and 235delC in Orientals. Carrier frequencies in these populations vary between 1 and 30 and 1 in 75. Over 50 mutations have been identified in the GJB2 gene, of which some missense changes have a dominant-negative action in hearing impairment, with partial to full penetrance. Functional studies for some missense mutations in connexins 26, 30, and 32 indicate abnormal gap junction conductivity. Expression patterns in mouse and rat cochlea indicate that connexin 26 and connexin 30 are expressed in the supporting cells of the cochlea, suggesting a potential role in endolymph potassium recycling.

In the Japanese population, Kudo et al. (2000) sequenced the GJB2 gene in 39 patients with prelingual deafness, 39 patients with postlingual progressive sensorineural hearing loss, and 63 individuals with normal hearing. GJB2 mutations were found in 5 of the 39 patients (12%) with prelingual deafness. The most common mutation was 235delC observed in 7 of 10 mutant alleles. There were no cases with the 30delG allele. No GJB2 mutation was found in patients in the postlingual hearing loss group. Nance et al. (2000) noted that recessive mutations at the connexin-26 gene locus account for nearly half of all cases of genetic deafness in many populations. They suggested that this high frequency is only seen in populations with a long tradition of intermarriage among deaf people. Available data are consistent with the hypothesis that such marriages might well have contributed to the high frequency of connexin-26 deafness in the U.S., and could represent a novel mechanism for maintaining specific genotypes at unexpectedly high frequencies.

The NOV58 disclosed in this invention is predicted to be expressed in at least the following tissues: brain, lung, ovary, and colon. This information was derived by determining the tissue sources of the sequences that were included in the invention including but not limited to SeqCalling sources, public EST sources, literature sources, and/or RACE sources. Further expression data for NOV58 is provided in Example 2.

The nucleic acids and proteins of NOV58 are useful in potential therapeutic applications implicated in various gap junction-related pathological disorders described further herein. The NOV58 nucleic acid encoding the connexin-like protein of the invention, or fragments thereof, may further be useful in diagnostic applications, wherein the presence or amount of the nucleic acid or the protein are to be assessed. The novel nucleic acid of the invention encoding a Connexin-like protein includes the nucleic acid whose sequence is provided in Table 58A or 58C, or a fragment thereof. The invention also includes a mutant or variant nucleic acid any of whose bases may be changed from the corresponding base shown in Table 58A or 58C while still encoding a protein that maintains its connexin-like activities and physiological functions, or a fragment of such a nucleic acid. The invention further includes nucleic acids whose sequences are complementary to the sequence shown in Table 58A or 58C, including nucleic acid fragments that are complementary to any of the nucleic acids just described. The invention additionally includes nucleic acids or nucleic acid fragments, or complements thereto, whose structures include chemical modifications. Such modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 29% of the bases may be so changed. The novel protein of the invention includes the connexin-like protein whose sequence is provided in Table 58B or 58D. The invention also includes a mutant or variant protein any of whose residues may be changed from the corresponding residue shown in Table 58B or 58D while still encoding a protein that maintains its connexin-like activities and physiological functions, or a functional fragment thereof. In the mutant or variant protein, up to about 44% of the amino acid residues may be so changed.

These materials are further useful in the generation of antibodies that bind immunospecifically to the substances of the invention for use in therapeutic or diagnostic methods. These antibodies may be generated according to methods known in the art, using prediction from hydrophobicity charts, as described in the “Anti-NOVX Antibodies” section below.

NOV59

A eukaryotic translation initiation factor 5 (EIF5), disclosed herein as NOV59, interacts with the 40S initiation complex to promote hydrolysis of bound GTP with concomitant joining of the 60S ribosomal subunit to the 40S initiation complex. The resulting functional 80S ribosomal initiation complex is then active in peptidyl transfer and chain elongations. The disclosed NOV59 (alternatively referred to herein as CG56633-01) includes the 1328 nucleotide sequence (SEQ ID NO:213) shown in Table 59A. A NOV59 ORF begins with a Kozak consensus ATG initiation codon at nucleotides 34-36 and ends with a TGA codon at nucleotides 1273-1275. The disclosed NOV59 maps to human chromosome 3.

TABLE 59A
NOV59 Nucleotide Sequence

| | |
|---|---|
| CTTTCCTCATCACCTTAAATTCGGGTGTCTTTTATGAGTAATCAAAAGCAGCAAAAGCCA | (SEQ ID NO:213) |
| ACGCTATCAGGCCCAGTATTTAAAACCAGAAAAAGAGATGAAAAAGAGAGGTTTGACCCT | |
| ACTCAGTTTCAGGACTACGTTATTCAAGGCTTAACTGAAACTGGTACTGATTTGGAAGCA | |
| GTAGCTAAGTTCCTTGATGCTTCTGGAACAAAACTTGATTACCGTCGATGTGCAGAAACA | |
| CTCTTTGACATTCTGGTGGGTGGTGGAATGCTGGCCCCAGGTGGTACACTGGCAGATGAC | |
| ATCATGCGTACAGATGTCTGCGTGTTTGCAGCCCAAGAAGACCTAGAGACCATGCAAGCA | |
| TTTGCTCAGGTTTTTAACAAGTTAATCAGGCACTACAAATACCTTGAGAAATGTTGTGAA | |
| GATGAAGTAAAAAGGCTGCTGGTGTTCGGAAAGGGTTTTTCAGACTCGGAGAGGAAAAAA | |
| CTGGCTATGTTGACTGGTGTTCTTCTGGCTAATGCATCCATTCTTAATAGCCTTTATAAT | |
| GAGAATTTGGTTAAAGAAGGGGTTTCAACAGCTTTTGCTGGAAAGCTATTTAAATCATGT | |
| ATAAATGAAAAAGATATCAATGCAGTAACTGCAAGGAAAGTCAGCATGGATAACAGCCTG | |
| ATGGAACTTTTTCCTGCCAATAAGCAAAGCGTTCAACACTTCACGAAGTATTTTACTGAG | |
| GCAGGCCTGAAAGAGCTTTCAGAATATGTTCGGAATCAGCAAACCATCAGAGCTTGTAAG | |
| GAGCTGCAGAAAGAACTTCAAGAACAGATGTCCCGTGGGGATCCATTTAAGGTTATAATT | |
| TTATATGTCAAGGAGGAGATGAAAAAAAACAACATCCCAGAACCAGTTGTCATCGAAATA | |
| GTCTGGTCAAATGTAATGAGCGCTGTGGAATGGAACAAAAGAGAGGAGATTGTAGCAGAG | |
| CAAGCCATCAAACACTTGAAGCAACACAGCCCTCTACTTGCTGCCTTTACTACTCAAAGT | |
| CAGTCTGAGCTGACCCTGTTACTGAAGATTCAGGAGTATTGCTATGACAACATTCATTTC | |
| ATGAAAGCCTTACGGAAAATAGTGGTGCTTTTTTATAAAGCTGTAGTCCTGAGCAAAGAG | |
| ACCATTTTGAAGTGGTATAAAGGTACACATGTTGCAAAGGGGAAGAGTGTTTTCCTTGAG | |
| CAAATGAAAAAGTTTGGAGAGTGGCTCAAAAATGCTGAAGAAGAATCTGAATCTGAAGCT | |
| GAAGAAGGTGACTGAATTTTGAAACTACACCCTCAGTAAAGCAAACAGGAGTTGTAGATA | |
| AAATGTCC | |

The NOV59 polypeptide (SEQ ID NO:214) encoded by SEQ ID NO:213 is 413 amino acids in length and is presented using the one-letter amino acid code in Table 59B. The Psort profile for NOV59 predicts that this sequence has no signal peptide and is likely to be localized to the nucleus with a certainty of 0.7600. In alternative embodiments, a NOV59 polypeptide is located to lysosomes with a certainty of 0.10000.

TABLE 59B
NOV59 Polypeptide Sequence

| | |
|---|---|
| MSNQKQQKPTLSGPVFKTRKRDEKERFDPTQFQDYVIQGLTETGTDLEAVAKFLDASGTK | (SEQ ID NO:214) |
| LDYRRCAETLFDILVGGGMLAPGGTLADDIMRTDVCVFAAQEDLETMQAFAQVFNKLIRH | |
| YKYLEKCCEDEVKRLLVFGKGFSDSERKKLAMLTGVLLANASILNSLYNENLVKEGVSTA | |
| FAGKLFKSCINEKDINAVTARKVSMDNSLMELFPANKQSVQHFTKYFTEAGLKELSEYVR | |
| NQQTIRACKELQKELQEQMSRGDPFKVIILYVKEEMKKNNIPEPVVIEIVWSNVMSAVEW | |
| NKREEIVAEQAIKHLKQHSPLLAAFTTQSQSELTLLLKIQEYCYDNIHFMKALRKIVVLF | |
| YKAVVLSKETILKWYKGTHVAKGKSVFLEQMKKFGEWLKNAEEESESEAEEGD | |

A BLAST analysis of NOV59 was run against the proprietary PatP GENESEQ Protein Patent database. It was found, for example, that the amino acid sequence of NOV59 had high homology to other proteins as shown in Table 59C.

TABLE 59C
BLASTX results from PatP database for NOV59

| | | Smallest |
|---|---|---|
| | | Sum |
| | High | Probability |
| Sequences producing High-scoring Segment Pairs: | Score | P(N) |
| patp:AAB43883 Human cancer associated protein | 1834 | 5.6e−189 |
| sequence | | |
| patp:AAW93950 Human regulatory molecule | 1403 | 2.6e−143 |
| HRM-6 protein | | |
| patp:AAB92726 Human protein sequence | 1403 | 2.6e−143 |
| patp:AAM38764 Human polypeptide | 1403 | 2.6e−143 |
| patp:AAM40550 Human polypeptide | 1403 | 2.6e−143 |

In a search of sequence databases, it was found, for example, that the nucleic acid sequence of this invention has 778 of 824 bases (94%) identical to a gb:GENBANK-ID:HUMRSC419|acc:D13630.1 mRNA from Homo sapiens (Human mRNA for KIAA0005 gene. The full amino acid sequence of the protein of the invention was found to have 372 of 419 amino acid residues (88%) identical to, and 385 of 419 amino acid residues (91%) similar to, the 419 amino acid residue ptnr:SPTREMBL-ACC:Q15394 protein from Homo sapiens (Human) (KIAA0005 PROTEIN). NOV59 also has homology to the other proteins shown in the BLASTP data in Table 59D.

TABLE 59D
NOV59 BLASTP results

| Gene Index/ | | Length | Identity | Positive | |
|---|---|---|---|---|---|
| Identifier | Protein/Organism | (aa) | (%) | (%) | Expect |
| gi\|7661850\|ref\| | basic leucine-zipper | 419 | 372/419 | 385/419 | 0.0 |
| NP_055485.1\| | protein BZAP45; KIAA0005 | | (88) | (91) | |
| (NM_014670) | gene product [Homo | | | | |
| | sapiens] | | | | |
| gi\|7661744\|ref\| | HSPC028 protein | 419 | 264/406 | 355/406 | e−143 |
| NP_054757.1\| | [Homo sapiens] | | (65) | (82) | |
| (NM_014038) | | | | | |
| gi\|153417861\|gb\| | HSPC028 protein | 419 | 264/406 | 334/406 | e−143 |
| AAH13060.1\| | [Mus musculus] | | (65) | (82) | |
| AAH13060 | | | | | |
| (BC013060) | | | | | |
| gi\|4426565\|gb\| | unknown | 419 | 264/406 | 334/406 | e−143 |
| AAD20436.1\| | [Rattus norvegicus] | | (65) | (82) | |
| (AF031483) | | | | | |
| gi\|11640562\|gb\| | MSTP017 | 419 | 263/406 | 334/406 | e−143 |
| AAG39278.1\| | [Homo sapiens] | | (64) | (81) | |
| AF110323_1 | | | | | |
| (AF110323) | | | | | |

This BLASTP data is displayed graphically in the ClustalW in Table 59E. A multiple sequence alignment is given, with the NOV59 protein being shown on line 1 in a ClustalW analysis comparing the protein of the invention with the related protein sequences shown in Table 59D.

[image]

[image]

Table 59F lists the domain description from DOMAIN analysis results against NOV59. This indicates that the NOV59 sequence has properties similar to those of other proteins known to contain this domain.

TABLE 59F
Domain Analysis of NOV59

gnl|Load|LOAD W2, W2, conserved protein-protein interaction domain in
translationfactors like eIF2B SEQ ID NO:860
CD-Length = 116 residues, 96.6% aligned
Score = 83.6 bits (205), Expect = 2e−17

| | | | |
|---|---|---|---|
| NOV59: | 290 | VWSNVMSAVEWNKREEIVAEQAIKHLKQHSPLLAAFTTQSQSELTLLLKIQEYCYDNIHF | 349 |
| | | V ++S + E A+K K+ PLLA + S+L LL ++E+C + | |
| Sbjct: | 1 | VALVILSVASIELADNEPKEAAVKVFKKWGPLLAKYLKDEDSQLELLYALEEFCEELEEL | 60 |
| NOV59: | 350 | MKALRKIVVLFYKAVVLSKETILKWY-KGTHVAKGKSVFLEQMKKFGEWLKN 400 | |
| | | +K L KI+ Y VL +E ILKWY K + +GK L+ K F WL+ | |
| Sbjct: | 61 | LKLLAKILKYLYDEDVLEEEAILKWYEKKSKAEEGKKKVLKSAKPFVTWLQE 112 | |

The NOV59 disclosed in this invention is predicted to be expressed in at least the following tissues: brain. This information was derived by determining the tissue sources of the sequences that were included in the invention including but not limited to SeqCalling sources, public EST sources, literature sources, and/or RACE sources. Further expression data for NOV59 is provided in Example 2.

The nucleic acids and proteins of NOV59 are useful in potential therapeutic applications implicated in various pathological disorders described further herein, for example, Von Hippel-Lindau (VHL) syndrome, Alzheimer's disease, stroke, tuberous sclerosis, hypercalceimia, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, myasthenia gravis, neuroprotection, endocrine dysfunctions, diabetes, obesity, growth and reproductive disorders and other diseases, disorders and conditions of the like. The NOV59 nucleic acid encoding the translation initiation factor 5-like protein of the invention, or fragments thereof, may further be useful in diagnostic applications, wherein the presence or amount of the nucleic acid or the protein are to be assessed. The novel nucleic acid of the invention encoding a translation initiation factor 5-like protein includes the nucleic acid whose sequence is provided in Table 59A, or a fragment thereof. The invention also includes a mutant or variant nucleic acid any of whose bases may be changed from the corresponding base shown in Table 59A while still encoding a protein that maintains its translation initiation factor 5-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 6% of the residues may be so changed.

The novel protein of the invention includes the translation initiation factor 5-like protein whose sequence is provided in Table 59B. The invention also includes a mutant or variant protein any of whose residues may be changed from the corresponding residue shown in Table 59b while still encoding a protein that maintains its translation initiation factor 5-like activities and physiological functions, or a functional fragment thereof. In the mutant or variant protein, up to about 12% of the bases may be so changed.

These materials are further useful in the generation of antibodies that bind immunospecifically to the substances of the invention for use in therapeutic or diagnostic methods. These antibodies may be generated according to methods known in the art, using prediction from hydrophobicity charts, as described in the “Anti-NOVX Antibodies” section below.

NOV60

NOV60 includes two Lynx1-like proteins, designated herein as NOV60a and NOV60b, which differ by three amino acids and the relative length of their untranslated regions (UTR's).

NOV60a

The disclosed NOV60a (alternatively referred to herein as CG56894-01) includes the 715 nucleotide sequence (SEQ ID NO:215) shown in Table 60A. A NOV60a ORF begins with a Kozak consensus ATG initiation codon at nucleotides 348-350 and ends with a stop codon at nucleotides 696-698.

TABLE 60A
NOV60a Nucleotide Sequence

(SEQ ID NO:215)

AGCTTTGTTCTTGAGTGGGTCTGCCTCGGGGGCTTTAGAGGAGACCCCAGAGGGTGGCGATGCGGCACGG
GTGCTGCGGGACACACAGACACGCCTACGATTAGACTCAGGCAGGCACCTACCGGCGAGCGGCCGCGGGT
GACTCCCAGGCGCGGCGGTACCTCACGGTGGTGAAGGTCACAGGGTTGCAGCACTCCCAGTAGACCAGGA
GCTCCGGGAGGCAGGGCCGGCCCCACGTCCTCTGCGCACCACCCTGAGTTGGATCCTCTGTGCGCCACCC
CTGAGTTGGATCCAGGGCTAGCTGCTGTTGACCTCCCCACTCCCACGCTGCCCTCCTGCCTGCAGCCATG
ACGCCCCTGCTCACCCTGATCCTGGTGGTCCTCATGGGCTTACCTCTGGCCCAGGCCTCGGACTGCCACG
TGTGTGCCTACAACGGAGACAACTGCTTCAACCCCATGCGCTGCCCGGCTATGGTTGCCTACTGCATGAC
CACGCGCACCTACTACACCCCCACCAGGATGAAGGTCAGTAAGTCCTGCGTGCCCCGCTGCTTCGAGACT
GTGTATGATGGCTACTCCAAGCACGCGTCCACCACCTCCTGCTGCCAGTACGACCTCTGCAACGGCACCG
GCCTTGCCACCCCGGCCACCCCGGCCCTGGCCCCCATCCTCCTGGCCACCCTCTGGGGTCTCCTCTAAAG
CCCCCGAGGCAGACA

The NOV60a polypeptide (SEQ ID NO:216) encoded by SEQ ID NO:215 is 116 amino acids in length and is presented using the one-letter amino acid code in Table 60B. The Psort profile for NOV60a predicts that this sequence has a signal peptide and is likely to be exported from the cell with a certainty of 0.8200. In alternative embodiments, a NOV60a polypeptide is located to lysosomes with a certainty of 0.1000, or to the endoplasmic reticulum (membrane) with a certainty of 0.1000. The Signal P predicts a likely cleavage site for a NOV60a peptide is between positions 34 and 35, i.e., at the dash in the sequence AQA-SD.

TABLE 60B
NOV60a Polypeptide Sequence

| | |
|---|---|
| MTPLLTLILVVLMGLPLAQASDCHVCAYNGDNCFNPMRCPAMVAYCMTTRTYYTPTRMKV | (SEQ ID NO:216) |
| SKSCVPRCFETVYDGYSKHASTTSCCQYDLCNGTGLATPATPALAPILLATLWGLL | |

NOV60b

The disclosed NOV60b (alternatively referred to herein as CG56894-02) includes the 876 nucleotide sequence (SEQ ID NO: ______) shown in Table 60C. A SEC2 ORF begins with a Kozak consensus ATG initiation codon at nucleotides 348-350 and ends with a stop codon at nucleotides 696-698.

TABLE 60C
NOV60b Nucleotide Sequence

| | |
|---|---|
| AGCTTTGTTCTTGAGTGGGTCTGCCTCGGGGGCTTTAGAGGAGACCCCAGAGGGTGGCGA | (SEQ ID NO:217) |
| TGCGGCACGGGTGCTGCGGGACACACAGACACGCCTACGATTAGACTCAGGCAGGCACCT | |
| ACCGGCGAGCGGCCGCGGGTGACTCCCAGGCGCGGCGGTACCTCACGGTGGTGAAGGTCA | |
| CAGGGTTGCAGCACTCCCAGTAGACCAGGAGCTCCGGGAGGCAGGGCCGGCCCCACGTCC | |
| TCTGCGCACCACCCTGAGTTGGATCCTCTGTGCGCCACCCCTGAGTTGGATCCAGGGCTA | |
| GCTGCTGTTGACCTCCCCACTCCCACGCTGCCCTCCTGCCTGCAGCCATGACGCCCCTGC | |
| TCACCCTGATCCTGGTGGTCCTCATGGGCTTACCTCTGGCCCAGGCCTCGGACTGCCACG | |
| TGTGTGCCTACAACGGAGACAACTGCTTCAACCCCATGCGCTGCCCGGCTATGGTTGCCT | |
| ACTGCATGACCACGCGCACCTACTACACCCCCACCAGGATGAAGGTCAGTAAGTCCTGCG | |
| TGCCCCGCTGCTTCGAGACTGTGTATGATGGCTACTCCAAGCACGCGTCCACCACCTCCT | |
| GCTGCCAGTACGACCTCTGCAACGGCACCGGCCTTGCCACCCCGGCCACCCTGGCCCTGG | |
| CCCCCATCCTCCTGGCCACCCTCTGGGGTCTCCTCTAAAGCCCCCGAGGCAGACCCACTC | |
| AAGAACAAAGCTCTCGAGACACACTGCTACACCCTCGCACCCAGCTCACCCTGCCTCACC | |
| CTCCACACTCCCTGCGACCTCCTCAAGCCATGCCCAGGGTCAGGACTGTGGGCAAGAAGAC | |
| ACCCGACCTCCCCCAACCACCACACGACCTCACTTC | |

The NOV60b polypeptide (SEQ ID NO:218) encoded by SEQ ID NO:217 is 116 amino acids in length and is presented using the one-letter amino acid code in Table 60D. The Psort profile for NOV60b predicts that this sequence is a Type Ia membrane protein, has a signal peptide, and is likely to be localized at the plasma membrane with a certainty of 0.9190. In alternative embodiments, a NOV60b polypeptide is located to lysosomes with a certainty of 0.2000, or to the endoplasmic reticulum (membrane) with a certainty of0.1000. The Signal P predicts a likely cleavage site for a NOV60b peptide is between positions 20 and 21, i.e., at the dash in the sequence AQA-SD.

TABLE 60D
NOV60b Polypeptide Sequence

| | |
|---|---|
| MTPLLTLILVVLMGLPLAQASDCHVCAYNGDNCFNPMRCPAMVAYCMTTRTYYTPTRMKV | (SEQ ID NO:218) |
| SKSCVPRCFETVYDGYSKHASTTSCCQYDLCNGTGLATPATLALAPILLATLWGLL | |

A BLAST analysis of NOV60 was run against the proprietary PatP GENESEQ Protein Patent database. It was found, for example, that the amino acid sequence of NOV60 had high homology to other proteins as shown in Table 60E.

TABLE 60E
BLASTX results from PatP database for NOV60

| | | Smallest |
|---|---|---|
| | | Sum |
| | High | Probability |
| Sequences producing High-scoring Segment Pairs: | Score | P(N) |
| patp:AAY02738 Human secreted protein encoded | 630 | 2.2e−61 |
| by gene 89 | | |
| patp:AAM39828 Human polypeptide | 630 | 2.2e−61 |
| patp:AAM41614 Human polypeptide | 630 | 2.2e−61 |
| patp:AAB61131 Human NOV3 protein | 594 | 1.4e−57 |
| patp:AAY79325 Mouse receptor ligand Lynx1 | 521 | 7.7e−50 |

In a search of sequence databases, it was found, for example, that the nucleic acid sequence of this invention has 338 of 424 bases (79%) identical to a gb:GENBANK-ID:AF141377|acc:AF141377.1 mRNA from Mus musculus (Ly-6/neurotoxin homolog (Lynx1) mRNA). The full amino acid sequence of the protein of the invention was found to have 92 of 116 amino acid residues (79%) identical to, and 96 of 116 amino acid residues (82%) similar to, the 116 amino acid residue ptnr:SPTREMBL-ACC:Q9WVC2 protein from Mus musculus (Mouse) (LY-6/NEUROTOXIN HOMOLOG). NOV60 also has homology to the other proteins shown in the BLASTP data in Table 60F.

TABLE 60F
NOV60 BLASTP results

| Gene Index/ | | Length | Identity | Positive | |
|---|---|---|---|---|---|
| Identifier | Protein/Organism | (aa) | (%) | (%) | Expect |
| gi\|7106349\|ref\| | Ly6/neurotoxin 1 | 116 | 92/116 | 96/116 | 5e−39 |
| NP_035968.1\| | [Mus musculus] | | (79%) | (82%) | |
| (NM_011838) | | | | | |
| gi\|12698684\| | Ly-6 neurotoxin- | 80 | 79/80 | 79/80 | 5e−27 |
| gb\| | like | | (98%) | (98%) | |
| AAK01642.1\| | protein Lynx1 | | | | |
| AF321824_1 | [Homo sapiens] | | | | |
| (AF321824) | | | | | |
| gi\|1519481\|gb\| | E48 antigen | 79 | 28/72 | 34/72 | 0.035 |
| AAB07524.1\| | [Homo sapiens] | | (38%) | (46%) | |
| (U66837) | | | | | |
| gi\|10720241\|sp\| | E48 antigen | 123 | 30/104 | 40/104 | 0.038 |
| P57096\| | [Homo sapiens] | | (28%) | (37%) | |
| PSCA_{—} | | | | | |
| MOUSE E48 | | | | | |
| antigen [Homo | | | | | |
| sapiens] | | | | | |
| gi\|12845967\| | PAR/Ly-6 | 154 | 36/108 | 47/108 | 0.068 |
| dbj\| | domain | | (33%) | (43%) | |
| BAB26976.1\| | containing protein | | | | |
| (AK010485) | [Mus musculus] | | | | |

This BLASTP data is displayed graphically in the ClustalW in Table 60G. A multiple sequence alignment is given, with the NOV60a and b protein being shown on lines 1 and 2 in a ClustalW analysis comparing the protein of the invention with the related protein sequences shown in Table 60F.

[image]

[image]

Elapid snake venom neurotoxins exert their effects through high-affinity interactions with specific neurotransmitter receptors. The lynx1-like gene disclosed herein as NOV60, is highly expressed in the brain and contains the cysteine-rich motif characteristic of this class of neurotoxins. Primary sequence and gene structure analyses reveal an evolutionary relationship between lynx1 and the Ly-6/neurotoxin gene family. Lynx1 is expressed in large projection neurons in the hippocampus, cortex, and cerebellum. In cerebellar neurons, lynx1 protein is localized to a specific subdomain including the soma and proximal dendrites. Lynx1 binding to brain sections correlates with the distribution of nAChRs, and application of lynx1 to Xenopus oocytes expressing nAChRs results in an increase in acetylcholine-evoked macroscopic currents. These results identify NOV60 as a protein modulator for nAChRs in vitro, with important implications in the regulation of cholinergic function in vivo.

The NOV60 disclosed in this invention is predicted to be expressed in at least the following tissues: brain. This information was derived by determining the tissue sources of the sequences that were included in the invention including but not limited to SeqCalling sources, public EST sources, literature sources, and/or RACE sources. Further expression data for NOV60 is provided in Example 2.

The nucleic acids and proteins of NOV60 are useful in potential therapeutic applications implicated in various pathological disorders described further herein, for example, the compositions of the present invention will have efficacy for the treatment of patients suffering from: Von Hippel-Lindau (VHL) syndrome, Alzheimer's disease, stroke, tuberous sclerosis, hypercalceimia, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, and neurodegeneration, as well as other diseases, disorders and conditions. The NOV60 nucleic acid encoding the lynx1-like protein of the invention, or fragments thereof, may further be useful in diagnostic applications, wherein the presence or amount of the nucleic acid or the protein are to be assessed. The nucleic acid of the invention encoding a lynx1-like protein includes the nucleic acid whose sequence is provided in Table 60A or 60C, or a fragment thereof.

The invention also includes a mutant or variant nucleic acid any of whose bases may be changed from the corresponding base shown in Table 60A or 60C while still encoding a protein that maintains its Lynx1-like activities and physiological functions, or a fragment of such a nucleic acid. The invention further includes nucleic acids whose sequences are complementary to the sequence of Table 60A or 60C including nucleic acid fragments that are complementary to any of the nucleic acids just described. The invention additionally includes nucleic acids or nucleic acid fragments, or complements thereto, whose structures include chemical modifications. Such modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 21% of the bases may be so changed.

The novel protein of the invention includes the lynx1-like protein whose sequence is provided in Table 60B and 60D. The invention also includes a mutant or variant protein any of whose residues may be changed from the corresponding residue shown in Table 60B and 60D while still encoding a protein that maintains its lynx1-like activities and physiological functions, or a functional fragment thereof. In the mutant or variant protein, up to about 21% of the amino acid residues may be so changed.

These materials are further useful in the generation of antibodies that bind immunospecifically to the substances of the invention for use in therapeutic or diagnostic methods. These antibodies may be generated according to methods known in the art, using prediction from hydrophobicity charts, as described in the “Anti-NOVX Antibodies” section below.

NOV61

NOV61 is a homolog of the adlican gene and belongs to the superfamily of cell adhesion molecules. The disclosed NOV61 (alternatively referred to herein as CG56453-01) includes the 5925 nucleotide sequence (SEQ ID NO: ______) shown in Table 61A. A NOV61 ORF begins with a Kozak consensus ATG initiation codon at nucleotides 16-18 and ends with a stop codon at nucleotides 5653-5655. The disclosed NOV70 maps to human chromosome Y.

TABLE 61A
NOV61 Nucleotide Sequence

| | |
|---|---|
| AGGCACCCCGACAAGATGCCCAAGCGCGCGCACTGGGGGGCCCTCTCTGTGGTGCTGATC | (SEQ ID NO:219) |
| CTGCTTTGGGGTCATCCGCGAGTGGCGCTGGCCTGCCCTCATCCTTGTGCCTGCTACGTC | |
| CCCAGCGAGGTCCACTGCACGTTCCGATCCCTGGCTTCTGTGCCCGCTGCCATTGCTAAA | |
| CATGTGGAAAGAATCAATTTGGGGTTTGGAATTCTGAAGTGTAAAAAGGACAAAGCTTAT | |
| GAAGGCGGTCAGTTGTGTGCAATGTGCTTCAGTCCAAAGAAGTTGTACAAACATGAGATT | |
| CACAAGCTCAAGGACCTGACTTGTCTGAAGCCTTCCATAGAGTCTCCTCTGAGACAGAAC | |
| AGGAGCAGGAGTATTGAGGAGGAGCAAAAACAAGAAGAGAATGGTGACAGCCAGCTCATC | |
| CTGGAGAAAATCCAACTTCCCCAGTGGAGCATCTCTTTGAATATGACTGATGAGCACGGG | |
| AACCTGGTGAACTTGGTGTGTGACATCAAGAAACCAATGGATGTGTACAAAATTCACTTG | |
| AACCAAACAGATCCTCCACATATTGACATAAATGCAATGGTTGCCTTGGACTTTGAGTAT | |
| CCAATGACCCAGGAAAACTATGAAAATCTATGGAAATTGATAGCATACTACAGTGAAGTT | |
| CCCATGAAGCTACACAGAGAGCTCATGCTCAGCAAACACCCCAGAGTCAGCTACCAGTAC | |
| AGGCAACATGCCGATGAAGAAGCTCTTTACTACACAGGTGTGAGAGCCCAGATTCTTGCA | |
| GAACCAGAATGGATCATGCAGCCATCCATAGATATCCAGCTGAACCGACCTCAGAGTACG | |
| GCCAAGAAGGTGCTACTTTCCTACTACAACCAGTATTCTCAAACAATAGCCACCAAAGAT | |
| ACAAGGCAGGCTCGGGGCAGAAGCTCGGTAATGATTGAGCCTAGTAGAGCTGTGCAAAAA | |
| GATCAGACTGTCCTGGAAGGGGGTCGATGCCAGTTGAGCTCCAATGTGAAAGCTTCTGAG | |
| AGTCCATCTATCTTCTGGCTGCTTCCAGATGGCTCCATCCTGAAAGTGCCTGTGGATGAC | |
| CCAGACAGCAAGTTCTCCATTCTCAGCAGTGGCTGGCTGAGGATCAAGTCCATGGAGCCA | |
| TCTGACTCGGGCTTGTACCAGTGCATTGCTCAAGTGAGGGATGAAATGGACCGCATGGTA | |
| TATAGGGTACTTGTGCAGTCTCCCTCCACTCAGCCAGCCGAGAAAGACACAGTGACAATT | |
| GGCAAGAACCCAGGGGAGCCAGTGATGTTGCCTTGCAATGCTTTAGCTATACCCGAAGCC | |
| CACCTTAGCTGGATTCTTCCAAACAGAAGGATAATTAATGATTTGGCTAACACATCACAT | |
| GTATACATGCTGCCAAATGGAACTCTTTCCATCCCAAAGGTCCAAGTCAGTGACAGTGGT | |
| TACCACAGATGTGTGGCTGTCAACCAGCATGGGGCAGACCATATCACGGTGGGAATCACA | |
| GTGACCAAGAAAGGTTCTGGCTCGCCATCCAAAAGAGGCAGATGGCCAGGTCCAAAGGCT | |
| CTTTCCAGAATGAGAGAAGACATCGTGGAGGATGAAGGGGTCTCAGGCACGGGAGATGAA | |
| GAGAACACTTCAAGGAGACTTCTACATCCAAAGCACCAAGAGGCGTTCCTCAAAACAAAG | |
| GATGATGCCATCAATGGAGATAAGAAAGCCAAGAAAGGGAGAAGAAAGCTGAAACTCTGG | |
| AAGCATTCAGAAAAAGAACCAGACACCAGTGTTGCAGAAGATCTCAGAGTGTTTGAATCA | |
| AGACGAAGGATAAACGTGGCAAACAAACAGATTAATCCGGAGCACTGGGCTGATATTTTA | |
| GCCAAAGTCTTTGGGAAAAATCTCCCTACAGGCACAGAAGTATCCCCAATTATTAAAACC | |
| ACAAGTTCTCCATTCTTGAGCCTAGTAGTCACACCACCTTTGCCTGCTGTTTCTCCCCCC | |
| TTGGCATCTCCAATACAGACAGCAACAAGTGCTGAAGAATCCTCAGCAGATGTACCTCTA | |
| CTCAGCGAAGGAAAGCACATTTTGAGTACCATTTCCTCACCCAGCATGGGACTAGAACAC | |
| CACAACAATGGAGTTATTCTTGTTGAACCTGAAGTAACAAGCACACCTCTGGAAGAAGTT | |
| GTTGATGAGTATTCCAAGAAGACTGAGGAGATGACTTCCACTGAAGGCGACCTGAAGGGG | |
| ACTGCAGCCTCTACACTTATATCTGACCCTTATGAACAATCTCCTACTCTACACACCTTA | |
| GACACAGTCTATGAAGAGCCCACCCATGAAGAGACGGAAACAGAGGGTTGGTCTGCAGCA | |
| GATGTTGGATCCTCACCACATCCCACATCCAGTGAGTATGAGCTTCCATTGGTTGTTGTC | |
| TCCTTGGCTGAGTCTAAGCCTGTGCAATACTTTGACCCAGATTTGGAGACTAATTCACAA | |
| CCACATGAGGATAACATAAAAGAATACAGTTTTGCACACCTTACTCCAACCGCCATCATC | |
| TGGTTTAATGACTCTACTACATCACTGTCATTTGAGGATTCTACTGTAGGGGAACAAGGT | |
| GTCCCAGGCAAATCACATCTACAAGGACCGACAGAGAACATCCAGCTTGTGAAAAGTACT | |
| TTTAGCACTCAAGACACCTTATTCATTAAAAAAGGTATGAAAGAGATGTCTCAGACACTA | |
| CAGCGAGGAAATATGCTAGAGGGAGACCCTACACACTCCAGAAGTTCTGAGAATGAGGGC | |
| CAAGAGAGCAAATCCATCACTTTACCTGACTCCACACTGGGTATAACGAGCAGTACGTCT | |
| CCAGTTAAGAAGCCTGCGGAAACCACAGTTGTCACCCTGCTACACAAAGACACCACAACA | |
| GAAACAACTCCAAGGCAAAAAGTGGCTTCATCATCCACCATGAGCACTCACCCTTCTCGA | |
| AGGAGACCCAATGGGAGAAAATTACACCCTCACAAATTCCACCACCGGCACAAGCAAACC | |
| CCACCCACAACTTTTGCTCCATTAGACACTTTTTCTACTCAACCAACTCAAGCAACTGAC | |
| ATTAAGATTTCAAATCAAATGGAGAGTTCTCTGGTTCCTACATCTTGGGAGATTAACACA | |
| GTTAATACCCCCAAACAGCTGGAAATGGAGAAGAATGTAGAGCTCATATCAAAGGGAACT | |
| CCACGGAGAAAACACGGGAAGAGGCCAAACAAACATCGATATACCCCTTCTACAGTGAGT | |
| TCAAGAGCATCTGCATCCAAGCCCAGCCCTTCTCCAGAAAATAAACATAGAAACATTGTT | |
| ACTCCCAGTTCAGAAACTACACTTTTGCCTAGAAATGTTTCTCTGAAAACTGAGGGCGTT | |
| TATGATTCCTTAGATTACACGACAACCACCAGAAAAATACATTCATCTCACCATAAAGTC | |
| CAAGACACACTTCCAGTCATGTATAAACCCACATCAGATCGAAAACAAATTCAGGATGAT | |
| GTTGCCACAAATGTTGACAAACATAAAAGTGACATTTTAGTCCCTGGTGAGTCAATTACA | |
| AATGTCACACAAACTTCTCGCTCCTTGGTCTCCACTATGGGAGAATTTAAGGAAGAATCC | |
| TCTCCTGTGGGCTTTCCAGGAATTCCAACCTGGAATCCCTCAAGGAAAGCTCAGCCTGGG | |
| AGGCTACAGACAGACATACATGTTACCACTTCTGGGGAAACCCCTACAGACCCTCCCCTT | |
| GTTAACGAGCTTGAGGATGTGCATTTTACTTCTGAGTTTTTGTCCTCTGTGACAGTCTCC | |
| ACACCATTTCACCACGAAGAAGCTGGTTTTTCCACAATTCTCTCAAGCATAAAAGTGGAG | |
| ATGGCTTCAAGTCAGGTAGAAACTACCACCCTTGGTCAAGATCATCATGAAACCACTGTG | |
| GCTATTCTCCACTCTGAAACTAGACCACAGAATCACATCCTTACTGCTGCCTGGATCAAG | |
| GAGCCAGCATCTTTGTCCCCTCCCATGATTCTCCTGTCTTTGGGACAAACCACCACCACT | |
| AAGCCAGAACTTCTCAGTCCAAGAACATCTCAAATATGTAAAGATTCCAAGGAAAATGTT | |
| TTCTTGAATTACATGGGGAATCCAGAAACAGAAGCAACCCCAGTGAAAAATGAAGGAACA | |
| CAGCGTATGTCAGGGCCAAATGAATTATCAACACCATCTTCTGACCACGATGCATTTAAC | |
| TTGTCTACAAAGCTAGAATTGGAAAAGCAAGTATTTGATAGTAGGAGTCTAACACGTGGC | |
| CCAGATAGCCACCACCAGGATGGAAGAGTTCATGCTTCTCATCAACTAACCAGAATCCCT | |
| GCCAAACCCATCCTACCAACAGGAACAGTGAGGCTGCCTGAAATGTCCACACAAAGCACT | |
| TCCAGATACTTTGTAACTTTCCAGCCACCTCATCACGGGACCAACAAACCAGAAATAACT | |
| ACATATCCTTCTAGGGCTTTGCCAGAGAGCAAACAGTTTACAACTCCAAGAGTAGCAAGT | |
| ACAACTCCTCTCCTATCACACATGTCCAAACCCAGCATTTCTAGTAAGTTTGCTGACCTA | |
| AGAACTGACCAATCCAATGGCTCCTACAAAGTGTTTGGAAATAGCAACATCCCTGAGGCA | |
| AGAAACTCAGTTGGAAAGCCTCTCAGTCCAAGAATTTATCATTATTCCAATGGAAGACTC | |
| CCTTTCTTTACCAACAGGACTCTTTCTTTTTCACAGTTGGGAGTCACCCGGAGACCCCAG | |
| ATACCCTCTTCTCCTGTCCCAGTAATGAGAGAGAGAAAAGTTAATCCAGGTTCCTACAAT | |
| AGGATATATTCCCATAGCACCTTCCATCTGGACTTTGGCCTTCCAGCACCTCCACTGTTG | |
| CACACTCCATGGACCATGGTATCACCCCCAACTAACTTACAGAATATCCCTATGGTCTCA | |
| TCCACCCAGAGTTCTGTCTCCTTTATAACATCTTCTGTCCAGTCCTCAGGAAGCATCCAC | |
| CAAAGCGGCTCAAAGTTCTTTGCAGGAGGACCGCCTGCATCCAAATTCTGGCCTCTTGGG | |
| GAAAAGCCCCAAATCCTCACCAAGTCCCCACAGACTGTGTCTGTCACTGCTGAAACGGAC | |
| GCTGTGTTCCCGTGTGAGGCAATAGGAAAACCAAAGCCTTTCGTTACTTGGACAAAAGTT | |
| TCCACAGGAGTTCTTATGACTCCGAATACCAGGATACAACGGTTTGAGGTTCTCAAGAAC | |
| GGTACCTTAGTGATAAGGAAGTTTCAAGTGCAAGATCGAGGCCAGTATATGTGCACCGCC | |
| AGCAACCTGTACGGCCTGGACAGGATGCTGGTCTTTCTCTCGGTCACCGTGCAGCAACCT | |
| CAAATCCTAGCCTCCCACTACCAGGACGTCACCGTCTACCTGGGAGACACCATTACAATG | |
| GAGTGTCTGGCGAAAGGGACCCCAGCCCCCCAAATTTCCTGGATCTTCCGTGACAGGAGG | |
| GTGTGGCAAACTCTGTCCTCCGTGGAGGGCCGGATCACCCTGCACCAAAACCGGACCCTT | |
| TCCATCAAGGAGGCGTCCTTCTCAGACAGAGGCGTCTATAAGTGCGTGGCCAGCAACGCA | |
| ACCCGGGCGGACAGCGTGTCCATCCGCCTACACGTGGCGGCACTGCCCCCCATTATCCAC | |
| CAGGAGAAGCTGTAGAACATCTCGCTGCCCCCGGGGCTCAGCATTCACATTCACTGCACT | |
| GCCAAAGCTGCGCCCCTGCCCAGCGTGCTCTGGGTGCTCGGGGATGGTACCCAAATCCGC | |
| CCCTCGCATTTCCTCCACCGGAACTTGTTTGTTTTCCCCAACGGGACGCTCTACATCTGC | |
| AACCTCGCGCCCAACGACAGCGGGCGCTATGAGTGCGTGGCCGCCAACCTGATCGGCTCC | |
| GCGCGCAGTACGGTGCAGCTGAACGTGCAGCGCGCAGCAGCGAAC | |

The NOV61 polypeptide (SEQ ID NO:220) encoded by SEQ ID NO:219 is 1879 amino acids in length and is presented using the one-letter amino acid code in Table 61B. The Psort profile for NOV61 predicts that this sequence has a signal peptide and is likely to be localized outside the cell with a certainty of0.4371. In alternative embodiments, a NOV61 polypeptide is located to lysosomes with a certainty of 0.1900, to the endoplasmic reticulum (membrane) with a certainty of 0.1000, or to the nucleus with a certainty of 0.1800. The Signal P predicts a likely cleavage site for a NOV61 peptide is between positions 26 and 27, i.e., at the dash in the sequence ALA-CP.

TABLE 61B
NOV61 Polypeptide Sequence

| | |
|---|---|
| MPKRAHWGALSVVLILLWGHPRVALACPHPCACYVPSEVHCTFRSLASVPAGIAKHVERI | (SEQ ID NO:220) |
| NLGFGILKCKKDKAYEGGQLCANCFSPKKLYKHEIHKLKDLTCLKPSIESPLRQNRSRSI | |
| EEEQKQEEMGDSQLILEKIQLPQWSISLNMTDEHGNLVNLVCDIKKPMDVYKIHLNQTDP | |
| PDIDINAMVALDFEYPMTQENYENLWKLIAYYSEVPMKLHRELMLSKHPRVSYQYRQDAD | |
| EEALYYTGVRAQILAEPEWIMQPSIDIQLNRPQSTAKKVLLSYYNQYSQTIATKDTRQAR | |
| GRSWVMIEPSRAVQKDQTVLEGGRCQLSCNVKASESPSIFWVLPDGSILKVPVDDPDSKF | |
| SILSSCWLRIKSMEPSDSGLYQCIAQVRDEMDRMVYRVLVQSPSTQPAEKDTVTIGKNPG | |
| EPVMLPCNALAIPEAHLSWILPNRRIINDLANTSHVYMLPNGTLSIPKVQVSDSGYHRCV | |
| AVNQHGADHITVGITVTKKGSGSPSKRGRWPGPKALSRNREDIVEDEGVSGTGDEENTSR | |
| RLLHPKHQEAFLKTKDDAINGDKKAKKGRRKLKLWKHSEKEPETSVAEDLRVFESRRRIN | |
| VANKQINPEHWADILAKVFGKNLPTCTEVSPIIKTTSSPFLSLVVTPPLPAVSPPLASPI | |
| QTATSAEESSADVPLLSEGKHILSTISSASMGLEHHNNGVILVEPEVTSTPLEEVVDEYS | |
| KKTEEMTSTEGDLKGTAASTLISEPYEQSPTLHTLDTVYEEPThEETETEGWSAADVGSS | |
| PDPTSSEYELPLVVVSLAESKPVQYFDPDLETNSQPHEDNIKEYSFAHLTPTAIIWENDS | |
| STSLSFEDSTVGEQGVPGKSHLQGPTENIQLVKSSFSTQDTLLIKKGMKEMSQTLQGGNM | |
| LEGDPTHSRSSENEGQESKSITLPDSTLGITSSTSPVKKPAETTVVTLLHKDTTTETTPR | |
| QKVASSSTMSTHPSRRRPNGRKLHPHKFHHRHKQTPPTTFAPLETFSTQPTQATDIKISN | |
| QMESSLVPTSWEINTVNTPKQLEMEKNVELISKGTPRRKHGKRPNKHRYTPSTVSSRASA | |
| SKPSPSPENKHRNIVTPSSETTLLPRNVSLKTEGVYDSLDYTTTTRKIHSSHHKVQDTLP | |
| VMYKPTSDCKEIQDDVATNVDKHKSDILVPGESITNVTQTSRSLVSTMGEFKEESSPVGF | |
| PGIPTWNPSRKAQPGRLQTDIHVTTSGETPTDPPLVNELEDVDFTSEFLSSVTVSTPFHQ | |
| EEAGFSTILSSIKVEMASSQVETTTLGQDHHETTVAILHSETRPQNHILTAAWMKEPASL | |
| SPPMILLSLGQTTTTKPELLSPRTSQICKDSKENVFLNYMGNPETEATPVKNEGTQRMSG | |
| PNELSTPSSDHDAFNLSTKLELEKQVFDSRSLTRGPDSHHQDGRVHASHQLTRIPAKPIL | |
| PTGTVRLPEMSTQSTSRYFVTFQPPHHGTNKPEITTYPSRALPESKQFTTPRVASTTPLL | |
| SHMSKPSISSKFADLRTDQSNGSYKVFGNSNIPEARNSVGKPLSPRIYHYSNGRLPFFTN | |
| RTLSFSQLGVTRRPQIPSSPVPVMRERKVNPGSYNRIYSHSTFHLDFGLPAPPLLHTPWT | |
| MVSPPTNLQNIPMVSSTQSSVSFITSSVQSSGSIHQSGSKFFAGGPPASKFWPLGEKPQI | |
| LTKSPQTVSVTAETDAVFPCEAIGKPKPFVTWTKVSTGVLMTPNTRIQRFEVLKNGTLVI | |
| RKFQVQDRCQYMCTASNLYGLDRMVVFLWVTVQQPQILASHYQDVTVYLGDTITMECLAK | |
| GTPAPQISWIFRDRRVWQTLSSVEGRITLHQNRTLSIKEASFSDRGVYKCVASNATRADS | |
| VSIRLHVAALPPIIHQEKL | |

A BLAST analysis of NOV61 was run against the proprietary PatP GENESEQ Protein Patent database. It was found, for example, that the amino acid sequence of NOV61 had high homology to other proteins as shown in Table 61C.

TABLE 61C
BLASTX results from PatP database for NOV61

| | | Smallest |
|---|---|---|
| | | Sum |
| | High | Probability |
| Sequences producing High-scoring Segment Pairs: | Score | P(N) |
| patp:AAM03157 Peptide #1839 encoded by probe | 2631 | 2.0e−273 |
| patp:AAM15395 Peptide #1829 encoded by probe | 2631 | 2.0e−273 |
| patp:AAM27883 Peptide #1920 encoded by probe | 2631 | 2.0e−273 |
| patp:AAM55191 Human brain expressed single | 2631 | 2.0e−273 |
| exon probe | | |
| patp:AAM67586 Human bone marrow expressed | 2631 | 2.0e−273 |
| probe | | |

In a search of sequence databases, it was found, for example, that the nucleic acid sequence of this invention has 4045 of 4330 bases (93%) identical to a gb:GENBANK-ID:AF245505|acc:AF245505.1 mRNA from Homo sapiens (adlican mRNA). The full amino acid sequence of the protein of the invention was found to have 1598 of 1818 amino acid residues (87%) identical to, and 1661 of 1818 amino acid residues (91%) similar to, the 2828 amino acid residue ptnr:SPTREMBL-ACC:Q9NR99 protein from Homo sapiens (Human) (ADLICAN). NOV61 also has homology to the other proteins shown in the BLASTP data in Table 61D.

TABLE 61D
NOV61 BLASTP results

| Gene Index/ | | Length | Identity | Positive | |
|---|---|---|---|---|---|
| Identifier | Protein/Organism | (aa) | (%) | (%) | Expect |
| gi\|14766612\|ref\| | hypothetical protein | 2828 | 1590/1818 | 1654/1818 | 0.0 |
| XP_035465.1\| | XP_035465 | | (87) | (90) | |
| (XM_035465) | [Homo sapiens] | | | | |
| gi\|9280405\|gb\| | Adlican | 2828 | 1591/1818 | 1654/1818 | 0.0 |
| AAF86402.1\| | [Homo sapiens] | | (87) | (90) | |
| AF245505_1 (AF245505) | | | | | |

This BLASTP data is displayed graphically in the ClustalW in Table 61E. A multiple sequence alignment is given, with the NOV61 protein being shown on line 1 in a ClustalW analysis comparing the invention with the related protein sequences shown in Table 61D.

[image]

[image]

[image]

[image]

[image]

Table 61F lists the domain description from DOMAIN analysis results against NOV61. This indicates that the NOV61 sequence has properties similar to those of other proteins known to contain this domain.

TABLE 61F
Domain Analysis of NOV61

gnl|Smart|smart00409, IG, Immunoglobulin SEQ ID NO:861
CD-Length = 86 residues, 100.0% aligned
Score = 62.8 bits (151), Expect = 2e−10

| | | | |
|---|---|---|---|
| NOV61: | 1685 | PQTVSVTAETDAVFPCEAIGKPKPFVTWTKVSTGVLMTPNTRIQRFEVLKNGTLVIRKFQ | 1744 |
| | | P +V+V CEA G P P VTW K G L + R N TL I | |
| Sbjct: | 1 | PPSVTVKEGESVTLSCEASGNPPPTVTWYKQG-GKLLAESGRFSVSRSGGNSTLTISNVT | 59 |
| NOV61: | 1745 | VQDRGQYMCTASNLYGLDRMVVFLWVT 1771 | |
| | | +D G Y C A+N G L V | |
| Sbjct: | 60 | PEDSGTYTCAATNSSGSASSGTTLTVL 86 | |

The gene of invention is a close homolog of the adlican gene and belongs to the superfamily of cell adhesion molecules. Cell adhesion molecules mediate key aspects of development, differentiation, cellular plasticity and physiological function in a variety of tissues. In addition, they are central to a number of disease processes such as cancer. Adlican is a protein that has been described to be elevated in patients with osteoarthritis. Sequence analysis indicates that it is likely to be a secreted protein. A rat gene named mechanical stress-induced protein has been patented and shows significant similarity to adlican. This protein is elevated in osteoblasts subjected to mechanical stress and has been suggested to be effective in the prognosis, diagnosis or treatment of osteoarthritis. Since this family of proteins seems to have involvement in osteoarthritis, it follows that the protein of invention may share that characteristic.

The disclosed NOV61 of invention has two significant attributes—it is truncated relative to its homolog, adlican, and secondly, it maps to the Y chromosome. The first attribute is significant in that it is possible that the truncated adlican-like protein may play a dominant-negative role in the function of adlican. It is therefore possible that it may be constitute an effective treatment for osteoarthritis. The chromosomal localization is relevant because it is known that osteoarthritis has a higher frequency in women. It is possible, therefore, that the truncated protein may be nullifying the effect of adlican, if any, in males.

The NOV61 disclosed in this invention is predicted to be expressed in at least the following tissues: muscle, lymph. This information was derived by determining the tissue sources of the sequences that were included in the invention including but not limited to SeqCalling sources, public EST sources, literature sources, and/or RACE sources. Further expression data for NOV61 is provided in Example 2.

The nucleic acids and proteins of NOV61 are useful in potential therapeutic applications implicated in various pathological disorders described further herein, for example, the compositions of the present invention will have efficacy for the treatment of patients suffering from: osteoarthitis, asthma, allergy, muscular dystrophy, Lesch-Nyhan syndrome, myasthenia gravis, hemophilia, hypercoagulation, idiopathic thrombocytopenic purpura, autoimmune disease, allergies, immunodeficiencies, transplantation, graft versus host disease (GVHD), lymphaedema, cancer, tissue degeneration as well as other diseases, disorders and conditions. The NOV61 nucleic acid encoding the adlican-like protein of the invention, or fragments thereof, may further be useful in diagnostic applications, wherein the presence or amount of the nucleic acid or the protein are to be assessed. The novel nucleic acid of the invention encoding a adlican-like protein includes the nucleic acid whose sequence is provided in Table 61A, or a fragment thereof. The invention also includes a mutant or variant nucleic acid any of whose bases may be changed from the corresponding base shown in Table 61A while still encoding a protein that maintains its adlican-like activities and physiological functions, or a fragment of such a nucleic acid. The invention further includes nucleic acids whose sequences are complementary to the sequence of Table 61A, including nucleic acid fragments that are complementary to any of the nucleic acids just described. The invention additionally includes nucleic acids or nucleic acid fragments, or complements thereto, whose structures include chemical modifications. Such modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 7% of the bases may be so changed.

The novel protein of the invention includes the adlican-like protein whose sequence is provided in Table 61B. The invention also includes a mutant or variant protein any of whose residues may be changed from the corresponding residue shown in Table 61B while still encoding a protein that maintains its adlican-like activities and physiological functions, or a functional fragment thereof. In the mutant or variant protein, up to about 13% of the amino acid residues may be so changed.

These materials are further useful in the generation of antibodies that bind immunospecifically to the substances of the invention for use in therapeutic or diagnostic methods. These antibodies may be generated according to methods known in the art, using prediction from hydrophobicity charts, as described in the “Anti-NOVX Antibodies” section below.

NOV62

NOV62 has homology to neuropsin, an extracellular matrix serine protease. The disclosed NOV62 (alternatively referred to herein as CG56781-01) includes the 834 nucleotide sequence (SEQ ID NO:221) shown in Table 62A. A NOV62 ORF begins with a Kozak consensus ATG initiation codon at nucleotides 31-33 and ends with a TGA codon at nucleotides 808-810. The disclosed NOV62 maps to human chromosone 19.

TABLE 62A
NOV62 Nucleotide Sequence

| | |
|---|---|
| AGTCTTGCCTTCTTTTGAGCCTAAGTCATGAGTTGGATGTTCCTCAGAGATCTCCTGAGT | (SEQ ID NO:221) |
| GGAGTAAATAAATACTCCACTGGGACTGGATGGATTTGGCTGGCTGTCGTGTTTGTCTTC | |
| CGTTTGCTGGTCTACATGGTGGCAGCAGAGCACGTGTGCAAACATGAGCAGAAAGAGTTT | |
| GAGTGCAACAGTAGACAGCCCGGTTGCAAAAATGTGTGTTTTGATGACTTCTTCCCCATT | |
| TCCCAAGTCAGACTTTGGGCCTTACAACTGATAATGGTCTCCACACCTTCACTTCTGGTG | |
| GTTTTACATGTAGCCTATCATGACGGTACAGAGAAAAGGCACAGAAAGAAACTCTATGTC | |
| AGCCCAGGTACAATGGATGGGGGCCTATGGTACGCTTATCTTATCAGCCTCATTCTTAAA | |
| ACTGGTTTTGAAATTGGCTTCCTTGTTTTATTTTATAAGCTATATGATGGCTTTAGTGTT | |
| CCCTACCTTATAAAGTGTGATTTGAAGCCTTGTCCCAACACTGTGGACTGCTTCATCTCC | |
| AAACCCACTGAGAAGACGATCTTCATCCTCTTCTTGGTCATCACCTCATGCTTGTGTATT | |
| GTGTTGAATTTCATTGAACTGAGTTTTTTGGTTCTCAAGTGCTTTATTAAGTGCTGTCTC | |
| CAAAAATATTTAAAAAAACCTCAAGTCCTCAGTGTGTGAGTGCCACAGCCTCAGATATGT | |
| TGAATGTG | |

The NOV62 polypeptide (SEQ ID NO:222) encoded by SEQ ID NO:221 is 259 amino acids in length and is presented using the one-letter amino acid code in Table 62B. The Psort profile for NOV62 predicts that this sequence has a signal peptide and is likely to be localized to the endoplasmic reticulum (membrane) at the plasma membrane with a certainty of 0.5500. In alternative embodiments, a NOV62 polypeptide is located to lysosomes with a certainty of 0.2353, or to the endoplasmic reticulum (lumen) with a certainty of 0.1000. The Signal P predicts a likely cleavage site for a NOV62 peptide is between positions 28 and 29, i.e., at the dash in the sequence TRA-QG.

TABLE 62B
NOV62 Polypeptide Sequence

| | |
|---|---|
| MGRPPPCAIQPWILLLLFMGAWAGVTRAQGSRSRKGQASKPHSQPWQAALFQGERLICGG | (SEQ ID NO:222) |
| VLVGDRWVLTAAHCKKQKYSVRLGDHSLQSRDQPEQEIQVAQSIQHPCYNNSNPEDHSHD | |
| IMLIRLQNSANLGDKVKPVQLANLCPKVGQKCIISGWGTVTSPQENFPNTLNCAEVKIYS | |
| QNKCERAYPGKITEGMVCAGSSNGADTCQGDSGGPLVCEGTLAGIVSGGSEPVFRPRRPA | |
| VYTNVFDYLEWIESTMEKN | |

A BLAST analysis of NOV62 was run against the proprietary PatP GENESEQ Protein Patent database. It was found, for example, that the amino acid sequence of NOV62 had high homology to other proteins as shown in Table 62C.

TABLE 62C
BLASTX results from PatP database for NOV62

| | | Smallest |
|---|---|---|
| | | Sum |
| | High | Probability |
| Sequences producing High-scoring Segment Pairs: | Score | P(N) |
| patp:AAW10694 Human recombinant neuropsin | 1247 | 8.9e−127 |
| patp:AAW12393 Mouse neuropsin protein | 1247 | 8.9e−127 |
| patp:AAB21311 Human neuropsin - Homo sapiens, | 972 | 1.2e−97 |
| 275 aa. | | |
| patp:AAY41744 Human PRO322 protein sequence | 965 | 6.8e−97 |
| patp:AAY32852 Human serine protease protein | 965 | 6.8e−97 |
| sequence | | |

In a search of sequence databases, it was found, for example, that the nucleic acid sequence of this invention has 747 of 805 bases (92%) identical to a gb:GENBANK-ID:E12348|acc:E12348.1 mRNA from Mus sp. The full amino acid sequence of the protein of the invention was found to have 227 of 257 amino acid residues (88%) identical to, and 238 of 257 amino acid residues (92%) similar to, the 260 amino acid residue ptnr:SWISSNEW-ACC:Q61955 protein from Mus musculus (Mouse) (NEUROPSIN PRECURSOR (EC 3.4.21.) (NP)). NOV62 also has homology to the other proteins shown in the BLASTP data in Table 62D.

TABLE 62D
NOV62 BLASTP results

| Gene Index/ | | Length | Identity | Positive | |
|---|---|---|---|---|---|
| Identifier | Protein/Organism | (aa) | (%) | (%) | Expect |
| gi\|6679487\|ref\| | protease, serine, 19 | 260 | 227/257 | 238/257 | e−131 |
| NP_032966.1\| | (neuropsin); Brain | | (88) | (92) | |
| (NM_008940) | Serine protease 1 | | | | |
| | [Mus musculus] | | | | |
| gi\|6093538\|sp\| | NEUROPSIN PRECURSOR | 260 | 216/258 | 232/258 | e−126 |
| O88780\|NRPN_{—} | (NP) (BRAIN SERINE | | (83) | (89) | |
| RAT | PROTEASE 1) | | | | |
| gi\|4699764\|pdb\| | Chain A, Neuropsin, A | 225 | 197/223 | 206/223 | e−112 |
| 1NPM\|A | Serine Protease | | (88) | (92) | |
| | Expressed In The Limbic | | | | |
| | System Of Mouse Brain | | | | |
| gi\|6005844\|ref\| | kallikrein 8 | 260 | 172/253 | 207/253 | 8e−99 |
| NP_009127.1\| | (neuropsin/ovasin); | | (67) | (80) | |
| (NM_007196) | protease, serine, 19 | | | | |
| | (neuropsin/ovasin) | | | | |
| | [Homo sapiens] | | | | |
| gi\|16162680\|ref\| | hypothetical protein | 260 | 169/253 | 203/253 | 3e−96 |
| XP_057595.1\| | XP_057595 | | (66) | (79) | |
| (XM_057595) | [Homo sapiens] | | | | |

This BLASTP data is displayed graphically in the ClustalW in Table 62E. A multiple sequence alignment is given, with the NOV62 protein being shown on line 1 in a ClustalW analysis comparing the protein of the invention with the related protein sequences shown in Table 62D.

[image]

[image]

Table 62F lists the domain description from DOMAIN analysis results against NOV62. This indicates that the NOV62 sequence has properties similar to those of other proteins known to contain this domain.

TABLE 62F
Domain Analysis of NOV62

gnl|Smart|smart00020, Tryp_SPc, Trypsin-like serine protease; Many of these
are synthesised as inactive precursor zymogens that are cleaved during
limited proteolysis to generate their active forms. A few, however, are
active as single chain molecules, and others are inactive due to
substitutions of the catalytic triad residues. SEQ ID NO:812
CD-Length = 230 residues, 98.3% aligned
Score = 237 bits (604), Expect = 7e−64

| | | | |
|---|---|---|---|
| NOV62: | 36 | GQASKPHSQPWQAALF-QGERLICGGVLVGDRWVLTAAHC----KKQKYSVRLGDHSLQS | 90 |
| | | G + S PWQ +L +G R CGG L+ RWVLTAAHC VRLG H L S | |
| Sbjct: | 5 | GSEANIGSFPWQVSLQYRGGRHFCGGSLISPRWVLTAAHCVYGSAPSSIRVRLGSHDLSS | 64 |
| NOV62: | 91 | RDQPEQEIQVAQSIQHPCYNNSNPEDHSHDIMLIRLQNSANLGDKVKPVQLA--NLCPKV | 148 |
| | | + Q ++V++ I HP YN P + +DI L++L L D V+P+ L | |
| Sbjct: | 65 | GE-ETQTVKVSKVIVHPNYN---PSTYDNDIALLKLSEPVTLSDTVRPICLPSSGYNVPA | 120 |
| NOV62: | 149 | GQKCIISGWGTVTSPQENFPNTLNCAEVKIYSQNKCERAYPG--KITEGMVCAGSSN-GA | 205 |
| | | G C +SGWG + + P+TL V I S C RAY G IT+ M+CAG G | |
| Sbjct: | 121 | GTTCTVSGWGRTSESSGSLPDTLQEVNVPIVSNATCRRAYSGGPAITDNMLCAGGLEGGK | 180 |
| NOV62: | 206 | DTCQGDSGGPLVCE---GTLAGIVSGGSEPVFRPRRPAVYTNVFDYLEWI 252 | |
| | | D CQGDSGGPLVC L GIVS GS RP +P VYT V YL+WI | |
| Sbjct: | 181 | DACQGDSGGPLVCNDPRWVLVGIVSWGSYGCARPNKPGVYTRVSSYLDWT 230 | |

Neuropsin appears to act as a regulatory molecule in the early phase of LTP via its proteolytic function on extracellular matrix rather than affecting NMDA receptor-mediated calcium increase. The behavioral and electrographical abnormalities associated with seizures in epileptic (kindled) mice correspond with those of human epilepsy. In kindled mice, neuropsin was markedly increased in the hippocampus and cerebral cortices. A single intraventricular injection of monoclonal antibodies specific to neuropsin reduced or eliminated the epileptic pattern noted on electroencephalograms and, as a result markedly inhibited the progression of kindling.

Therefore, neuropsin appears to be a key protein controlling pathogenic events in the hippocampus, and thus neuropsin inhibitors might be useful for treatment of epilepsy. Neuropsin has two isoforms, which have been reported to be involved in hippocampal plasticity. The amino acid sequences of the two types of human neuropsin were identical, except that type 2 carried an insert of 45 amino acids at the C-terminus of the leader sequence. The essential three amino acids in the active site triad, His, Asp, and Ser, and the single putative N-glycosylation site were conserved in human and mouse neuropsin. Sequence analysis of the 946 bp genomic DNA spanning the region encoding the insertion sequence revealed that two isoforms were generated in human brain by alternative splicing. However, the mouse genomic sequence did not conserve the 3′ acceptor consensus sequence at the corresponding position, suggesting that type 2 neuropsin was a species-specific splice variant. When the open reading frames of human neuropsin were expressed in insect cells, both types of neuropsin were detected in the conditioned media by western blot analysis using anti-human neuropsin serum.

Northern blot hybridization and reverse transcription-polymerase chain reaction showed predominant expression of type 1 neuropsin in pancreas. Type 2 neuropsin was preferentially expressed in human adult brain and hippocampus, although both types were expressed in fetal brain and placenta in comparable amounts. Dot blot hybridization showed that neuropsin was expressed in various regions of adult brain, including the hippocampus and cerebral cortex, and also in various fetal tissues. These results suggest that human type 2 neuropsin may be important to the adult brain plasticity, although both types may be necessary for the development of the nervous system.

The disclosed NOV62 is predicted to be expressed in at least the following tissues: brain. This information was derived by determining the tissue sources of the sequences that were included in the invention including but not limited to SeqCalling sources, public EST sources, literature sources, and/or RACE sources. Further expression data for NOV62 is provided in Example 2.

The nucleic acids and proteins of NOV62 are useful in potential therapeutic applications implicated in various pathological disorders described further herein, for example, the compositions of the present invention will have efficacy for treatment of patients suffering from: Von Hippel-Lindau (VHL) syndrome, Alzheimer's disease, stroke, tuberous sclerosis, hypercalceimia, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, neuroprotection, osteoarthritis, and other diseases, disorders and conditions of the like. The NOV62 nucleic acid encoding the neuropsin-like protein of the invention, or fragments thereof, may further be useful in diagnostic applications, wherein the presence or amount of the nucleic acid or the protein are to be assessed. The novel nucleic acid of the invention encoding a neuropsin precursor-like protein includes the nucleic acid whose sequence is provided in Table 62A, or a fragment thereof. The invention also includes a mutant or variant nucleic acid any of whose bases may be changed from the corresponding base shown in Table 62A while still encoding a protein that maintains its neuropsin precursor-like activities and physiological functions, or a fragment of such a nucleic acid. The invention further includes nucleic acids whose sequences are complementary to those just described, including nucleic acid fragments that are complementary to any of the nucleic acids just described.

The invention additionally includes nucleic acids or nucleic acid fragments, or complements thereto, whose structures include chemical modifications. Such modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 8% of the residues may be so changed.

The novel protein of the invention includes the neuropsin precursor-like protein whose sequence is provided in Table 62B. The invention also includes a mutant or variant protein any of whose residues may be changed from the corresponding residue shown in Table 62B while still encoding a protein that maintains its neuropsin precursor-like activities and physiological functions, or a functional fragment thereof. In the mutant or variant protein, up to about 12% of the bases may be so changed.

These materials are further useful in the generation of antibodies that bind immunospecifically to the substances of the invention for use in therapeutic or diagnostic methods. These antibodies may be generated according to methods known in the art, using prediction from hydrophobicity charts, as described in the “Anti-NOVX Antibodies” section below.

NOV63

NOV63 has homology to the WNT family of proteins. The Wnt gene family consists of at least 15 structurally related genes that encode secreted extracellular signaling factors. WNT proteins function in a range of critical developmental processes in both vertebrates and invertebrates and are implicated in regulation of cell growth and differentiation in certain adult mammalian tissues, including the mammary gland. The disclosed NOV63 (alternatively referred to herein as CG56054-02) includes the 1128 nucleotide sequence (SEQ ID NO: ______) shown in Table 63A. A NOV63 ORF begins with a Kozak consensus ATG initiation codon at nucleotides 31-33 and ends with a stop codon at nucleotides 1102-1104. The disclosed NOV63 maps to human chromosome 1.

TABLE 63A
NOV63 Nucleotide Sequence

| | |
|---|---|
| TCCTCCCCGCAGCTTCTCGCTGAATTCCGAGGGGGCTGAGAGGATGGCCACCACCGGGAC | (SEQ ID NO:223) |
| CCCAACGGCCGACCGAGGCCACGCAGCCGCCACAGATGACCCGGCCGCCCGCTTCCAGGT | |
| GCAGAAGCACTCGTGGCACGGGCTCCGGAGCATCATCCACGGCAGCCGCAAGTACTCGGG | |
| CCTCATTGTCAACAAGGCGCCCCACGACTTCCAGTTTGTGCAGAAGACCGATGAGTCTGG | |
| GCCCCACTCCCACCGCCTCTACTACCTGGGAATGCCATATGGCACCCGAGAGAACTCCCT | |
| CCTCTACTCTGAGATTCCCAAGAAGGTCCGGAAAGAGGCTCTGCTGCTCCTGTCCTGGAA | |
| GCAGATGCTGGATCATTTCCAGGCCACGCCCCACCATGGGGTCTACTCTCGGGAGGAGGA | |
| GCTGCTGAGGGAGCGGAAACGCCTGGGGGTCTTCGGCATCACCTCCTACGACTTCCACAG | |
| CGAGAGTGGCCTCTTCCTCTTCCAGGCCAGCAACAGCCTCTTCCACTCCCGCGACGGCGG | |
| CAAGAACGGCTTCATGGTGTCCCCTATGAAACCGCTGCAAATCAAGACCCAGTGCTCAGG | |
| GCCCCGGATGGACCCCAAAATCTGCCCTGCCGACCCTGCCTTCTTCTCCTTCATCAATAA | |
| CAGCGACCTGTGGGTGGCCAACATCGAGACAGGCGAGGACCGGCGGCTGACCTTCTCCCA | |
| CCAAGGTTTATCCAATGTCCTGGATGACCCCAAGTCTGCGGGTGTGGCCACCTTCGTCAT | |
| ACAGGAAGAGTTCGACCGCTTCACTGGGTACTGCTGGTGCCCCACAGCCTCCTGGGAAGG | |
| TTCAGAGGGCCTCAAGACGCTGCGAATCCTGTATGAGGAAGTCGATGAGTCCGAGGTGGA | |
| GGTCATTCACGTCCCCTCTCCTGCGCTAGAAGAAAGGAAGACGGACTCGTATCGGTACCC | |
| CAGGACAGGTAGCAAGAATCCCAAGATTGCCTTCAAACTGGCTGAGTTCCAGACTGACAG | |
| CCAGGGCAAGATCGTCTCGACCCAGGAGAAGGAGCTGGTGCAGCCCTTCAGCTCGCTGTT | |
| CCCGAAGGTGGAGTACATCGCCAGGGCCGGGTGGACCCGGGATGGCAAATACGCCTGGGC | |
| CATGTTCCTGCACCGGCCCCAGCAGTGGCTCCAGCTCGTCCTCCTCCCCCCGGCCCTGTT | |
| CATCCCGAGCACAGAGAATGAGGAGCAGCGGCTAGCCTCTGCCAGAGCTGTCCCCAGGAA | |
| TGTCCAGCCGTATGTGGTGTACGAGGAGGTCACCAACGTCTGGATCAATGTTCATGACAT | |
| CTTCTATCCCTTCCCCCAATCAGAGGGAGAGGACGAGCTCTGCTTTCTCCGCGCCAATGA | |
| ATGCAAGACCGGCTTCTGCCATTTGTACAAAGTCACCGCCGTTTTAAAATCCCAGGGCTA | |
| CGATTGGAGTGAGCCCTTCAGCCCCGGGGAAGATGAATTTAAGTGCCCCATTAAGGAAGA | |
| GATTGCTCTGACCAGCGGTGAATGGGAGGTTTTGGCGAGGCACGGCTCCAAGATCTGGGT | |
| CAATGAGGAGACCAAGCTGGTGTACTTCCAGGGCACCAAGGACACGCCGCTGGAGCACCA | |
| CCTCTACGTGGTCAGCTATGAGGCGGCCGGCGAGATCGTACGCCTCACCACGCCCGGCTT | |
| CTCCCATAGCTGCTCCATCAGCCAGAACTTCGACATGTTCGTCAGCCACTACAGCAGCGT | |
| GAGCACGCCGCCCTGCGTGCACGTCTACAAGCTGAGCGGCCCCGACGACGACCCCCTGCA | |
| CAAGCAGCCCCGCTTCTGGGCTAGCATGATGGAGGCAGCCAGCTGCCCCCCGGATTATGT | |
| TCCTCCAGAGATCTTCCATTTCCACACGCGCTCGGATGTGCGGCTCTACGGCATGATCTA | |
| CAAGCCCCACGCCTTGCAGCCAGAGAAGAACCACCCCACCGTCCTCTTTGTATATGGAGG | |
| CCCCCAGGTGCAGCTGGTGAATAACTCCTTCAAAGGCATCAAGTACTTGCGGCTCAACAC | |
| ACTGGCCTCCCTGGGCTACGCCGTCGTTGTGATTGACGGCAGGGGCTCCTGTCAGCGAGG | |
| GCTTCGGTTCGAAGGGGCCCTGAAAAACCAAATGGGCCAGGTGGAGATCGAGGACCAGGT | |
| GGAGGGCCTGCAGTTCGTGGCCGAGAAGTATGGCTTCATCGACCTGAGCCGAGTTGCCAT | |
| CCATGGCTGGTCCTACGGGGGCTTCCTCTCGCTCATGGGGCTAATCCACAAGCCCCAGGT | |
| GTTCAAGGTGGCCATCGCGGGTGCCCCGGTCACCGTCTGGATGGCCTACGACACACGGTA | |
| CACTGAGCGCTACATGGACGTCCCTGAGAACAACCAGCACGGCTATGAGGCGGGTTCCGT | |
| GGCCCTGCACGTGGAGAAGCTGCCCAATGAACCCAACCGCTTGCTTATCCTCCACGGCTT | |
| CCTGGACGAAAACGTGCACTTTTTCCACACAAACTTCCTCGTCTCCCAACTGATCCGAGC | |
| AGGGAAACCTTACCAGCTCCAGATCTACCCCAACGAGAGACACAGTATTCGCTGCCCCGA | |
| GTCGGGCGAGCACTATGAAGTCACGTTGCTGCACTTTCTACAGGAATACCTCTGAGCCTG | |
| CCCACCGGGAGCCGCCACAT | |

The NOV63 polypeptide (SEQ ID NO:224) encoded by SEQ ID NO:223 is 357 amino acids in length and is presented using the one-letter amino acid code in Table 63B. The Psort profile for NOV63 predicts that this sequence has a signal peptide and is likely to be exported from the cell with a certainty of 0.3700. In alternative embodiments, a NOV63 polypeptide is located to lysosomes with a certainty of 0.1000, or to the endoplasmic reticulum (membrane) with a certainty of 0.1000. The Signal P predicts a likely cleavage site for a NOV63 peptide is between positions 18 and 19, i.e., at the dash in the sequence ALG-SY.

TABLE 63B
NOV63 Polypeptide Sequence

| | |
|---|---|
| MATTGTPTADRGDAAATDDPAARFQVQKHSWDGLRSIIHGSRKYSGLIVNKAPHDFQFVQ | (SEQ ID NO224) |
| KTDESGPHSHRLYYLGMPYGSRENSLLYSEIPKKVRKEALLLLSWKQMLDHFQATPHHGV | |
| YSREEELLRERKRLGVFGITSYDFHSESGLFLFQASNSLFHCRDGGKNGFMVSPMKPLEI | |
| KTQCSGPRMDPKICPADPAFFSFINNSDLWVANIETGEERRLTFCHQGLSNVLDDPKSAG | |
| VATFVIQEEFDRFTGYWWCPTASWEGSEGLKTLRILYEEVDESEVEVIHVPSPALEERKT | |
| DSYRYPRTGSKNPKIALKLAEFQTDSQGKIVSTQEKELVQPFSSLFPKVEYIARAGWTRD | |
| GKYAWANFLDRPQQWLQLVLLPPALFIPSTENEEQRLASARAVPRNVQPYVVYEEVTNVW | |
| INVHDIFYPFPQSEGEDELCFLRANECKTGFCHLYKVTAVLKSQGYDWSEPFSPGEDEFK | |
| CPIKEEIALTSGEWEVLARHGSKIWVNEETKLVYFQGTKDTPLEHHLYVVSYEAAGEIVR | |
| LTTPGFSHSCSMSQNFDMFVSHYSSVSTPPCVHVYKLSGPDDDPLHKQPRFWASMMEAAS | |
| CPPDYVPPEIFHFHTRSDVRLYQMIYKPHALQPEKKHPTVLFVYGGPQVQLVNNSFKGIK | |
| YLRLNTLASLGYAVVVIDGRGSCQRGLRFEQALKNQMGQVEIEDQVEGLQFVAEKYGFID | |
| LSRVAIHGWSYGGFLSLMGLIHKPQVFKVAIAGAPVTVWMAYDTGYTERYMDVPENNQHG | |
| YEAGSVALHVEKLPNEPNRLLILHGFLDENVHFFHTNFLVSQLIRAGKPYQLQIYPNERH | |
| SIRCPESGEHYEVTLLHFLQEYL | |

A BLAST analysis of NOV63 was run against the proprietary PatP GENESEQ Protein Patent database. It was found, for example, that the amino acid sequence of NOV63 had high homology to other proteins as shown in Table 63C.

TABLE 63C
BLASTX results from PatP database for NOV63

| | | Smallest |
|---|---|---|
| | | Sum |
| | High | Probability |
| Sequences producing High-scoring Segment Pairs: | Score | P(N) |
| patp: AAY81693 Human Wnt-6 protein sequence - | 411 | 1.0e−61 |
| Homo sapiens | | |
| patp: AAB49769 Amyloid-beta protein agglutination | 411 | 1.0e−61 |
| regulator | | |
| patp: AAB88439 Human membrane or secretory | 411 | 1.0e−61 |
| protein | | |
| patp: AAB19786 Human Wnt-1 protein involved | 630 | 2.2e−61 |
| in kidney | | |
| patp: AAY94319 Murine Wnt-10A protein - | 398 | 2.7e−61 |
| Mus musculus, 417 aa. | | |

In a search of sequence databases, it was found, for example, that the nucleic acid sequence of this invention has 633 of 795 bases (79%) identical to a gb:GENBANK-ID:AF031168|acc:AFO31168.1 mRNA from Gallus gallus (Gallus gallus Wnt-14 protein (Wnt-14) mRNA). The full amino acid sequence of the protein of the invention was found to have 287 of 349 amino acid residues (82%) identical to, and 319 of 349 amino acid residues (91%) similar to, the 354 amino acid residue ptnr:SWISSPROT-ACC:O42280 protein from Gallus gallus (Chicken) (WNT-14 PROTEIN PRECURSOR). NOV63 also has homology to the other proteins shown in the BLASTP data in Table 63D.

TABLE 63D
NOV63 BLASTP results

| Gene Index/ | | Length | Identity | Positive | |
|---|---|---|---|---|---|
| Identifier | Protein/Organism | (aa) | (%) | (%) | Expect |
| gi\|15082261\|ref\| | wingless-type MMTV | 365 | 339/356 | 343/356 | 0.0 |
| NP_003386.1\| | integration site family, | | (95) | (96) | |
| (NM_003395) | member 14 | | | | |
| | [Homo sapiens] | | | | |
| gi\|3915306\|sp\| | WNT-14 PROTEIN PRECURSOR | 354 | 283/333 | 310/333 | e−156 |
| O42280\|WN14_{—} | | | (84) | (92) | |
| CHICK | | | | | |
| gi\|163032641\|dbj\| | WNT14B | 357 | 215/352 | 263/352 | e−107 |
| BAB70499.1\| | [Homo sapiens] | | (61) | (74) | |
| (AB063483) | | | | | |
| gi\|17017976\|ref\| | wingless-type MMTV | 357 | 215/352 | 263/354 | e−107 |
| NP_003387.1\| | integration site family, | | (61) | (74) | |
| (NM_003396) | member 15 precursor | | | | |
| | [Homo sapiens] | | | | |
| gi\|18181917\|dbj\| | Wnt14b | 359 | 216/354 | 264/354 | e−106 |
| BAB83866.1\| | [Mus musculus] | | (61) | (74) | |
| (AB073819) | | | | | |

This BLASTP data is displayed graphically in the ClustalW in Table 63E. A multiple sequence alignment is given, with the disclosed NOV63 protein being shown on line 1 in a ClustalW analysis comparing the protein of the invention with the related protein sequences shown in Table 63D.

[image]

[image]

Table 63F lists the domain description from DOMAIN analysis results against NOV63. This indicates that the NOV63 sequence has properties similar to those of other proteins known to contain this domain.

TABLE 63F
Domain Analysis of NOV63

gnl|Pfam|pfam00110, wnt, wnt family. SEQ ID NO:862
CD-Length = 313 residues, 99.7% aligned
Score = 283 bits (725), Expect = 9e−78

| | | | |
|---|---|---|---|
| NOV63: | 51 | CDRLK-LERKQRRNCRRDPGVAETLVEAVSMSALECQFQFRFERWNCTLEGRYRASL--- | 106 |
| | | C L L +QR++CRR+P V ++ E ++ ECQ QFR RWNC+ R R | |
| Sbjct: | 2 | CRSLPGLSPRQRQLCRRNPDVMASVSEGAQLAIQECQHQFRGRRWNCSTLDRLRVVFGKV | 61 |
| NOV63: | 107 | LKRGFKETAFLYAISSAGLTHALAKACSAGRMERCTCDE-APDLENREAWQWGGCGDNLK | 165 |
| | | LK+G +ETAF+YAISSAG+ HA+ +ACS G +E C CD + +WQWGGC DN++ | |
| Sbjct: | 62 | LKKGTRETAFVYAISSAGVAHAVTRACSEGELESCGCDYKKGPGGPQGSWQWGGCSDNVE | 121 |
| NOV63: | 166 | YSSKFVKEFL-GRRSSKDLRARVDFHNNLVGVKVIKAGVETTCKCHGVSGSCTVRTCWRQ | 224 |
| | | + +F +EF+ R +D R+ ++ HNN G K +K+ + CKCHGVSGSC+++TCW | |
| Sbjct: | 122 | FGIRFSREFVDARERERDARSLMNLHNNEACRKAVKSHMRRECKCHGVSGSCSMKTCWLS | 181 |
| NOV63: | 225 | LAPFHEVGKHLKHKYETALKVGSTTNEAAGEAGAISPPRGRASGAGCSDPLPRTPELVH | 283 |
| | | L F VG LK KY+ A++V + G A + R SD LV+ | |
| Sbjct: | 182 | LPDFRAVCDALKDKYDGAIRVEPNKRGMGQGSAPRLVAKNPRFKPPTRSD------LVY | 234 |
| NOV63: | 284 | LDDSPSFCL--AGRFSPCTACRRC---HREKNCESICCCRGHNTQSRVVTRPCQCQVRW | 337 |
| | | L+DSP +C S GT GR C CE +CCCRG+NTQ T C C+ W | |
| Sbjct: | 235 | LEDSPDYCERDRSTCSLGTQGRVCNKTSKGLDGCELLCCGRGYNTQQVERTEKCNCKFHW | 294 |
| NOV63: | 338 | CCYVECRQCTQREEVYTCK 356 | |
| | | CCYV+C +C + EV+TCK | |
| Sbjct: | 295 | CCYVKCEECQEVVEVHTCK 313 | |

Wnt-1 (previously known as int-1) is a proto-oncogene induced by the integration of the mouse mammary tumor virus. It is thought to play a role in intercellular communication and seems to be a signalling molecule important in the development of the central nervous system (CNS). The sequence of wnt-1 is highly conserved in mammals, fish, and amphibians. Wnt-1 is a member of a large family of related proteins that are all thought to be developmental regulators. These proteins are known as wnt-2 (also known as irp), wnt-3, up to wnt-15. At least four members of this family are present in Drosophila. One of them, wingless (wg), is implicated in segmentation polarity. All these proteins share the following features characteristics of secretory proteins, a signal peptide, several potential N-glycosylation sites and 22 conserved cysteines that are probably involved in disulfide bonds. The Wnt proteins seem to adhere to the plasma membrane of the secreting cells and are therefore likely to signal over only few cell diameters.

The NOV63 disclosed in this invention is predicted to be expressed in at least the following tissues: brain This information was derived by determining the tissue sources of the sequences that were included in the invention including but not limited to SeqCalling sources, public EST sources, literature sources, and/or RACE sources. Further expression data for NOV63 is provided in Example 2.

The nucleic acids and proteins of NOV63 are useful in potential therapeutic applications implicated in various pathological disorders described further herein, for example, the compositions of the present invention will have efficacy for treatment of patients suffering from: Von Hippel-Lindau (VHL) syndrome, Alzheimer's disease, stroke, tuberous sclerosis, hypercalceimia, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, neuroprotection, osteoarthritis, and other diseases, disorders and conditions of the like. The NOV63 nucleic acid encoding the WNT-14 precursor-like protein of the invention, or fragments thereof, may further be useful in diagnostic applications, wherein the presence or amount of the nucleic acid or the protein are to be assessed. The novel nucleic acid of the invention encoding a WNT-14 precursor-like protein includes the nucleic acid whose sequence is provided in Table 63A, or a fragment thereof. The invention also includes a mutant or variant nucleic acid any of whose bases may be changed from the corresponding base shown in Table 63A while still encoding a protein that maintains its WNT-14 precursor-like activities and physiological functions, or a fragment of such a nucleic acid. The invention further includes nucleic acids whose sequences are complementary to the sequence indicated in Table 63A, including nucleic acid fragments that are complementary to any of the nucleic acids just described.

The invention additionally includes nucleic acids or nucleic acid fragments, or complements thereto, whose structures include chemical modifications. Such modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 21% of the bases may be so changed.

The novel protein of the invention includes the WNT-14 precursor-like protein whose sequence is provided in Table 63B. The invention also includes a mutant or variant protein any of whose residues may be changed from the corresponding residue shown in Table 63B while still encoding a protein that maintains its WNT-14 precursor-like activities and physiological functions, or a functional fragment thereof. In the mutant or variant protein, up to about 18% of the amino acid residues may be so changed.

These materials are further useful in the generation of antibodies that bind immunospecifically to the substances of the invention for use in therapeutic or diagnostic methods. These antibodies may be generated according to methods known in the art, using prediction from hydrophobicity charts, as described in the “Anti-NOVX Antibodies” section below.

NOV64

NOV64 has homology to dipeptidyl peptidase. The disclosed NOV64 (alternatively referred to herein as CG56884-01) includes the 2660 nucleotide sequence (SEQ ID NO:225) shown in Table 64A. A NOV64 ORF begins with a Kozak consensus ATG initiation codon at nucleotides 44-46 and ends with a stop codon at nucleotides 2633-2635. The disclosed NOV64 maps to human chromosome 17.

TABLE 64A
NOV64 Nucleotide Sequence

| | |
|---|---|
| TCCTCCCCGCACCTTCTCGCTGAATTCCGAGGGGGCTGAGAGGATGGCCACCACCGGGAC | (SEQ ID NO:225) |
| CCCAACGGCCGACCGAGGCGACGCAGCCGCCACAGATGACCCGGCCGCCCGCTTCCAGGT | |
| GCAGAAGCACTCGTGGGACGGGCTCCGGAGCATCATCCACGGCAGCCGCAAGTACTCGGG | |
| CCTCATTGTCAACAAGGCGCCCCACGACTTCCAGTTTGTGCAGAAGACGGATGAGTCTGG | |
| GCCCCACTCCCACCGCCTCTACTACCTGGGAATGCCATATGGCAGCCGAGAGAACTCCCT | |
| GCAGATGCTGGATCATTTCCAGGCCACGCCCCACCATGGGGTCTACTCTCGGGAGGAGGA | |
| GCTGCTGAGGGAGCGGAAACGCCTGGCGCTCTTCGGCATCACCTCCTACGACTTCCACAC | |
| CGAGAGTGGCCTCTTCCTCTTCCAGGCCAGCAACAGCCTCTTCCACTGCCGCGACGGCGG | |
| CAAGAACGGCTTCATGGTGTCCCCTATGAAACCGCTGGAAATCAAGACCCAGTGCTCAGG | |
| GCCCCGGATGGACCCCAAAATCTGCCCTGCCGACCCTGCCTTCTTCTCCTTCATCAATAA | |
| CAGCGACCTGTGGGTCGCCAACATCGAGACAGGCCAGGAGCGGCGGCTGACCTTCTGCCA | |
| CCAAGGTTTATCCAATGTCCTGGATGACCCCAAGTCTGCGGGTGTGGCCACCTTCGTCAT | |
| ACAGGAAGAGTTCGACCGCTTCACTGGGTACTGGTGGTGCCCCACAGCCTCCTGGGAAGG | |
| TTCAGAGGGCCTCAAGACGCTGCGAATCCTGTATGAGGAAGTCGATGAGTCCGAGGTCGA | |
| GGTCATTCACGTCCCCTCTCCTGCGCTAGAAGAAAGGAAGACGGACTCGTATCGGTACCC | |
| CAGGACAGGTAGCAAGAATCCCAAGATTGCCTTGAAACTGGCTGAGTTCCAGACTGACAG | |
| CCAGGGCAAGATCGTCTCGACCCAGGAGAAGGACCTGGTGCAGCCCTTCAGCTCGCTGTT | |
| CCCGAAGGTGGAGTACATCGCCAGGGCCCCGTGGACCCGGGATGGCAAATACGCCTGGGC | |
| CATGTTCCTGGACCGGCCCCAGCAGTGGCTCCAGCTCGTCCTCCTCCCCCCGGCCCTGTT | |
| CATCCCGAGCACAGAGAATGACGAGCAGCGGCTAGCCTCTGCCAGAGCTGTCCCCAGGAA | |
| TGTCCAGCCGTATGTGGTGTACGAGGAGGTCACCAACGTCTGGATCAATGTTCATGACAT | |
| CTTCTATCCCTTCCCCCAATCAGAGGGACAGGACGAGCTCTGCTTTCTCCGCGCCAATGA | |
| ATGCAAGACCGGCTTCTGCCATTTGTACAAAGTCACCGCCGTTTTAAAATCCCAGGGCTA | |
| CGATTGGAGTGAGCCCTTCAGCCCCGGGGAAGATGAATTTAAGTGCCCCATTAAGGAAGA | |
| GATTGCTCTGACCAGCGGTGAATGGGAGGTTTTGGCGAGGCACGGCTCCAAGATCTGGGT | |
| CAATGAGGAGACCAAGCTGGTGTACTTCCAGGGCACCAAGGACACGCCGCTGGAGCACCA | |
| CCTCTACGTGGTCAGCTATGAGGCGGCCGGCGAGATCGTACGCCTCACCACGCCCGGCTT | |
| CTCCCATAGCTGCTCCATGAGCCAGAACTTCGACATGTTCGTCAGCCACTACAGCAGCGT | |
| GAGCACGCCGCCCTGCGTGCACGTCTACAAGCTGAGCGGCCCCGACGACGACCCCCTGCA | |
| CAAGCAGCCCCGCTTCTGGGCTAGCATGATGGAGGCACCCAGCTGCCCCCCGGATTATGT | |
| TCCTCCAGAGATCTTCCATTTCCACACGCGCTCGGATGTGCGGCTCTACGGCATGATCTA | |
| CAAGCCCCACGCCTTGCAGCCAGAGAAGAAGCACCCCACCGTCCTCTTTGTATATGGAGG | |
| CCCCCAGGTGCAGCTGGTGAATAACTCCTTCAAAGGCATCAAGTACTTGCGGCTCAACAC | |
| ACTGGCCTCCCTGGGCTACGCCGTGGTTGTGATTGACGGCAGGGGCTCCTGTCAGCGAGG | |
| GCTTCGGTTCGAAGGGGCCCTGAAAAACCAAATGGGCCAGGTGGAGATCGAGGACCAGGT | |
| GGAGGGCCTGCAGTTCGTGGCCGAGAAGTATGGCTTCATCGACCTGAGCCGAGTTGCCAT | |
| CCATGCCTGGTCCTACGGGGGCTTCCTCTCGCTCATGGGGCTAATCCACAACCCCCAGGT | |
| GTTCAAGGTGGCCATCGCGGGTGCCCCGGTCACCGTCTGGATGGCCTACGACACAGGGTA | |
| CACTGAGCGCTACATGGACGTCCCTGAGAACAACCAGCACGGCTATGAGGCGGGTTCCGT | |
| GGCCCTGCACGTCCAGAAGCTGCCCAATGAACCCAACCGCTTGCTTATCCTCCACGGCTT | |
| CCTGGACCAAAACGTGCACTTTTTCCACACAAACTTCCTCGTCTCCCAACTGATCCGAGC | |
| AGGGAAACCTTACCAGCTCCAGATCTACCCCAACCAGACACACACTATTCGCTGCCCCGA | |
| GTCGGGCGAGCACTATGAAGTCACGTTGCTGCACTTTCTACAGGAATACCTCTGAGCCTG | |
| CCCACCGGGAGCCGCCACAT | |

A NOV64 polypeptide (SEQ ID NO:226) encoded by SEQ ID NO:225 is 863 amino acids in length and is presented using the one-letter amino acid code in Table 64B. The Psort profile for NOV64 predicts that this sequence has no signal peptide and is likely to be localized at peroxisomal microbodies with a certainty of 0.6400. In alternative embodiments, a NOV64 polypeptide is located to lysosomes with a certainty of 0.1000, or to the cytoplasm with a certainty of 0.4500.

TABLE 64B
NOV64 Polypeptide Sequence

| | |
|---|---|
| MATTGTPTADRGDAAATDDPAARFQVQKHSWDGLRSIIHGSRKYSGLIVNKAPHDFQFVQ | (SEQ ID NO:226) |
| KTDESGPHSHRLYYLGMPYGSRENSLLYSEIPKKVRKEALLLLSWKQMLDHFQATPHHGV | |
| YSREEELLRERKRLGVFGITSYDFHSESGLFLFQASNSLFHCRDGGKNGFMVSPMKPLEI | |
| KTQCSGPRMDPKICPADPAFFSFINNSDLWVANIETGEERRLTFCHQGLSNVLDDPKSAG | |
| VATFVIQEEFDRFTGYWWCPTASWEGSEGLKTLRILYEEVDESEVEVIHVPSPALEERKT | |
| DSYRYPRTGSKNPKIALKLAEFQTDSQGKIVSTQEKELVQPFSSLFPKVEYIARAGWTRD | |
| GKYAWAMFLDRPQQWLQLVLLPPALFIPSTENEEQRLASARAVPRNVQPYVVYEEVTNVW | |
| INVHDIFYPFPQSEGEDELCFLRANECKTGFCHLYKVTAVLKSQGYDWSEPFSPGEDEFK | |
| CPIKEEIALTSGEWEVLARHGSKIWVNEETKLVYFQGTKDTPLEHHLYVVSYEAAGEIVR | |
| LTTPGFSHSCSMSQNFDMFVSHYSSVSTPPCVHVYKLSGPDDDPLHKQPRFWASMMEAAS | |
| CPPDYVPPEIFHFHTRSDVRLYGMIYKPHALQPEKKHPTVLFVYGGPQVQLVNNSFKGIK | |
| YLRLNTLASLGYAVVVIDGRGSCQRGLRFEGALKNQMGQVEIEDQVEGLQFVAEKYGFID | |
| LSRVAIHGWSYGGFLSLMGLIHKPQVFKVAIAGAPVTVWMAYDTGYTERYMDVPENNQHG | |
| YEAGSVALHVEKLPNEPNRLLILHGFLDENVHFFHTNFLVSQLIRAGKPYQLQIYPNERH | |
| SIRCPESGEHYEVTLLHFLQEYL | |

A BLAST analysis of NOV64 was run against the proprietary PatP GENESEQ Protein Patent database. It was found, for example, that the amino acid sequence of NOV64 had high homology to other proteins as shown in Table 64C.

TABLE 64C
BLASTX results from PatP database for NOV64

| | | Smallest |
|---|---|---|
| | | Sum |
| | High | Probability |
| Sequences producing High-scoring Segment Pairs: | Score | P(N) |
| patp: AAB41626 Human ORFX ORF1390 | 3403 | 0.0 |
| polypeptide sequence | | |
| patp: AAM38724 Human polypeptide | 1987 | 0.0 |
| patp: AAM40510 Human polypeptide | 1823 | 0.0 |
| patp: AAB47187 Human DPP8 - | 2868 | 1.5e−298 |
| Homo sapiens, 882 aa. | | |
| patp: AAY90299 Human peptidase, | 2547 | 1.6e−264 |
| HPEP-16 protein sequence | | |

In a search of sequence databases, it was found, for example, that the nucleic acid sequence of this invention has 1601 of 2525 bases (63%) identical to a gb:GENBANK-ID:AF221634|acc:AF221634.1 mRNA from Homo sapiens (dipeptidyl peptidase 8 (DPP8) mRNA). The full amino acid sequence of the protein of the invention was found to have 521 of 856 amino acid residues (60%) identical to, and 657 of 856 amino acid residues (76%) similar to, the 882 amino acid residue ptnr:TREMBLNEW-ACC:AAG29766 protein from Homo sapiens (Human) (DIPEPTIDYL PEPTIDASE 8). NOV64 also has homology to the other proteins shown in the BLASTP data in Table 64D.

TABLE 64D
NOV64 BLASTP results

| Gene Index/ | | Length | Identity | Positive | |
|---|---|---|---|---|---|
| Identifier | Protein/Organism | (aa) | (%) | (%) | Expect |
| gi\|17865311\|gb\| | dipeptidyl peptidase 9 | 863 | 862/863 | 862/863 | 0.0 |
| AAL47179.1\| | [Homo sapiens] | | (99) | (99) | |
| AF452102_1 | | | | | |
| (AF452102) | | | | | |
| gi\|17483229\|ref\| | hypothetical protein | 689 | 688/689 | 688/689 | 0.0 |
| XP_035636.2\| | XP_035636 | | (99) | (99) | |
| (XM_035636) | [Homo sapiens] | | | | |
| gi\|12855335\|dbj\| | DIPEPTIDYL PEPTIDASE | 883 | 519/859 | 658/859 | 0.0 |
| BAB30295.1\| | 8˜putative [Mus | | (60) | (76) | |
| (AK016546) | musculus] | | | | |
| gi\|11095188\|gb\| | dipeptidyl peptidase 8 | 882 | 516/840 | 650/840 | 0.0 |
| AAG29766.1\| | [Homo sapiens] | | (61) | (76) | |
| AF221634_1 | | | | | |
| (AF221634) | | | | | |
| gi\|3513303\|gb\| | R26984_1 | 508 | 492/543 | 495/543 | 0.0 |
| AAC33801.1\| | [Homo sapiens] | | (90) | (90) | |
| (AC005594) | | | | | |

This BLASTP data is displayed graphically in the ClustalW in Table 64E. A multiple sequence alignment is given, with the NOV64 protein being shown on line 1 in a ClustalW analysis comparing the protein of the invention with the related protein sequences shown in Table 64D.

[image]

[image]

[image]

Table 64F lists the domain description from DOMAIN analysis results against NOV64. This indicates that the NOV64 sequence has properties similar to those of other proteins known to contain this domain.

TABLE 64F
Domain Analysis of NOV64

gnl|Pfam|pfam00930, DPPIV_N_term, Dipeptidyl peptidase IV (DPP IV) N-terminal
region. This family is an alignment of the region to the N-terminal side of
the active site. The Prosite motif does not correspond to this Pfam entry.
SEQ ID NO:863
CD-Length = 504 residues
Score = 112 bits (280), Expect = 9e−26

| | | | |
|---|---|---|---|
| NOV64: | 200 | FFSFINNSDLWVANIETCEERRLTFCHQGLSNVLDDPKSAGVATFVIQEEFDRFTG-YWW | 258 |
| | | +F+ +++L++ + +G ++T G SN + + G+ +V +EE WW | |
| Sbjct: | 122 | KLAFVRDNNLYIQKLPSGPAIQITT--DGKSNDIFN----GIPDWVYEEEILSTDYALWW | 175 |
| NOV64: | 259 | CPTASWEGSEGLKTLRILYEEVDESEVEVIHVPSPALEE--RKTDSYRYPRTGSKNPKIA | 316 |
| | | P + Y ++SEV VI P + + +YP+ G NP + | |
| Sbjct: | 176 | SPDGD----------FLAYLRFNDSEVPVIEYPFYTDDSQYPEDMEIKYPKAGDPNPTVK | 225 |
| NOV64: | 317 | LKLAEFQTDSQGKIVSTQEKELVQPFSSLFPKVEYIARAOWTRDGKYAWAMFLDRPQQWL | 376 |
| | | L + G VS + +SL YI R W + + A +L+R Q | |
| Sbjct: | 226 | LFVVNLAD---GASVSE-----IPLPASLASGDYYITRVAWVTNERLA-VQWLNRDQNIS | 276 |
| NOV64: | 377 | QLVLLPPALFIPSTENEEQRLASARAVPRNVQPYVVYEEVTNVWINVHDIFYPFPQSEGE | 436 |
| | | L L A +S V +N +E+ W+ + P +G | |
| Sbjct: | 277 | VLSLCDTA-------------SSTWNVVKN------FEDSETGWVETFNPSLPVFPLDGL | 317 |
| NOV64: | 437 | DELCFLRANECKTGFCHLYKVTAVLKSQGYDWSEPFSPGEDEFKCPIKEEIALTSGEWEV | 496 |
| | | +L ++ + G+ HL E + K PI ALT G WEV | |
| Sbjct: | 318 | SY--YLDISD-RDGYKHLAYY------------------ELDGKEPI----ALTKGNWEV | 352 |
| NOV64: | 497 | LARHGSKIWVNEETKLVYFQGTKDTPLEHHLYVVSY----EAAGEIVRLTTPGFSHSCSM | 552 |
| | | + + V+ +T VYF T++ E HLY +S + G+ +S S | |
| Sbjct: | 353 | T----NILGVDSKTDTVYFTATEEGSGERHLYSISLKGGKTTLSCQLDSERCGY-YSASF | 407 |
| NOV64: | 553 | SQNFDMFVSHYSSVSTPPCVHVYKLSGPDDDPLHKQPRFWASMME------AASCPPDYV | 606 |
| | | S N ++ YS P S D L +E A | |
| Sbjct: | 408 | SPNAKYYILTYSGPGVP---IQTLHSSNDTKELR--------TLEDNEALKKALKNYQLP | 456 |
| NOV64: | 607 | PPEIFHFHTRSDVRLYGMIYKPHALQPEKKHPTVLFVYGGPQVQLV 652 | |
| | | E + L + KP P KK+P + FVYGGP QV | |
| Sbjct: | 457 | SKEFGKIKLADGITLNYQMIKPANFDPSKKYPVLFFVYGGPGSQQV 502 | |

NOV64 is a member of the family of dipeptidyl peptidases (DPPs). This group of enzymes catalyzes the removal of dipeptides from the N termini of polypeptides. This novel gene has greatest homology to a recently discovered protein, DPP8 (Abbott et al., Eur J Biochem October 2000;267(20):6140-50). DPP8 in turn is related to DPP4, which is a cell surface peptidase involved in T-cell activation (Kahne et al., Int J Mol Med July 1990;4(1):3-15). Other members of the peptidase family have been targeted as putative drug targets, for instance, in situations where they might cleave polypeptides beneficial in the prevention or reduction of a disease condition.

The NOV64 disclosed in this invention is predicted to be expressed in at least the following tissues: bone, bone marrow, brain (cerebellum,substantia nigra, thalamus), bronchus, cartilage, cervix, chorionic villus, coronary artery, colon, breast, heart, kidney, liver, lung, lymph node, lymphoid tissue, ovary, placenta, pituitary gland, respiratory bronchiole, retina, skeletal muscle, skin, small intestine,spinal cord, spleen, testis, thymus, thyroid, umbilical vein, urinary bladder, vulva, adrenal gland/suprarenal gland, synovium/synovial membrane, and uterus. This information was derived by determining the tissue sources of the sequences that were included in the invention including but not limited to SeqCalling sources, public EST sources, literature sources, and/or RACE sources. Further expression data for NOV64 is provided in Example 2.

The nucleic acids and proteins of NOV64 are useful in potential therapeutic applications implicated in various pathological disorders described further herein, for example, the compositions of the present invention will have efficacy for treatment of patients suffering from: Von Hippel-Lindau (VHL) syndrome, Alzheimer's disease, stroke, tuberous sclerosis, hypercalceimia, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, neuroprotection, osteoarthritis, and other diseases, disorders and conditions of the like. A NOV64 nucleic acid encoding the dipeptidyl peptidase-like protein of the invention, or fragments thereof, may further be useful in diagnostic applications, wherein the presence or amount of the nucleic acid or the protein are to be assessed.

The novel nucleic acid of the invention encoding a dipeptidyl peptidase-like protein includes the nucleic acid whose sequence is provided in Table 64A, or a fragment thereof. The invention also includes a mutant or variant nucleic acid any of whose bases may be changed from the corresponding base shown in Table 64A while still encoding a protein that maintains its dipeptidyl peptidase-like activities and physiological functions, or a fragment of such a nucleic acid. The invention further includes nucleic acids whose sequences are complementary to the sequence of Table 64A, including nucleic acid fragments that are complementary to any of the nucleic acids just described. The invention additionally includes nucleic acids or nucleic acid fragments, or complements thereto, whose structures include chemical modifications. Such modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 37% of the bases may be so changed.

The novel protein of the invention includes the dipeptidyl peptidase-like protein whose sequence is provided in Table 64B. The invention also includes a mutant or variant protein any of whose residues may be changed from the corresponding residue shown in Table 64B while still encoding a protein that maintains its dipeptidyl peptidase-like activities and physiological functions, or a functional fragment thereof. In the mutant or variant protein, up to about 40% of the amino acid residues may be so changed.

These materials are further useful in the generation of antibodies that bind immunospecifically to the substances of the invention for use in therapeutic or diagnostic methods. These antibodies may be generated according to methods known in the art, using prediction from hydrophobicity charts, as described in the “Anti-NOVX Antibodies” section below.

NOV65

NOV65 includes two dual specificity phosphatase-like proteins, designated herein as NOV65a and NOV65b.

NOV65a

A disclosed NOV65a (alternatively referred to herein as CG56651-01) includes the 711 nucleotide sequence (SEQ ID NO: ______) shown in Table 65A. A NOV65a ORF begins with a Kozak consensus ATG initiation codon at nucleotides 1-3 and ends with a stop codon at nucleotides 652-654. The disclosed NOV65a maps to human chromosome 1.

TABLE 65A
NOV65a Nucleotide Sequence

| | |
|---|---|
| ATGCTTCCCAAACGCGTGAGGGAGAAGATGGATGACACCAGCCTCTATAATACGCCCTGT | (SEQ ID NO:227) |
| GTCCTGGACCTACAGCGGGCCCTGGTTCAGGATCGCCAAGAGGCGCCCTGGAATGAGGTG | |
| GATGAGGTCTGGCCCAATGTCTTCATAGCTGAGAAGAGTGTGGCTGTGAACAAGGGGAGG | |
| CTGAAGAGGCTGGGAATCACCCACATTCTGAATGCTGCGCATGGCACCGGCGTTTACACT | |
| GGCCCCGAATTCTACACTGGCCTGGAGATCCAGTACCTGGGTGTAGAGGTGGATGACTTT | |
| CCTCAGGTGGACATTTCCCAGCATTTCCGGAAGGCGTCTGAGTTCCTGGATCAGGCGCTG | |
| CTGACTTACAGAGGGAAAGTCCTGGTCAGCAGCGAAATGGGCATCAGCCGGTCAGCAGTG | |
| CTGGTGGTCGCCTACCTGATGATCTTCCACAACATGGCCATCCTGCAGGCTTTGATGACC | |
| GTGCGTAAGAAGCGGGCCATCTACCCCAATGAGGGCTTCCTGAAGCAGCTGCGGGAGCTC | |
| AATGAGAAGTTGATGAGGAGAGAGAAGAGGACTATGGCCGGGAGGGGGGATCAGCTGAGG | |
| CTGAGGAGGGCGAGCGCACTGGGAGCATGCTCGGGGCCAGAGTGCACGCCCTGACGGTGG | |
| AAGAGGAGGACGACAGCGCCAGCCACCTGAGTGGCTCCTCCCTGGGGAAGG | |

The NOV65a polypeptide (SEQ ID NO:228) encoded by SEQ ID NO:227 is 217 amino acids in length and is presented using the one-letter amino acid code in Table 65B. The Psort profile for NOV65a predicts that this sequence is likely to be a Type II membrane protein, and is likely to be localized at the plasma membrane with a certainty of 0.4400. In alternative embodiments, a NOV65a polypeptide is located to the endoplasmic reticulum (membrane) with a certainty of 0.8500, or to the nucleus with a certainty of 0.7400.

TABLE 65B
NOV65a Polypeptide Sequence

| | |
|---|---|
| MLPKRVREKMDDTSLYNTPCVLDLQRALVQDRQEAPWNEVDEVWPNVFIAEKSVAVNKGR | (SEQ ID NO:228) |
| LKRLGITHILNAAHGTGVYTGPEFYTGLEIQYLGVEVDDFPEVDISQHFRKASEFLDEAL | |
| LTYRGKVLVSSEMGISRSAVLVVAYLMIFHNMAILEALMTVRKKRAIYPNEGFLKQLREL | |
| NEKLMRREKRTMAGRGDQLRLRRARALGACSGPECTP | |

NOV65b

The disclosed NOV65b (alternatively referred to herein as CG56652-02) includes the 3212 nucleotide sequence (SEQ ID NO: ______) shown in Table 65C. A SEC2 ORF begins with a Kozak consensus ATG initiation codon at nucleotides 1-3 and ends with a stop codon at nucleotides 3193-3195. The disclosed NOV65b maps to human chromosome 1.

TABLE 65C
NOV6Sb Nucleotide Sequence

| | |
|---|---|
| ATGCTTCCCAAACGCGTGAGGGAGAAGATGGATGACACCAGCCTCTATAATACGCCCTGT | (SEQ ID NO:229) |
| GTCCTGGACCTACAGCGGGCCCTGGTTCAGGATCGCCAAGAGGCGCCCTGGAATGAGGTG | |
| GATGAGGTCTGGCCCAATGTCTTCATAGCTGAGAAGAGTGTGGCTGTGAACAAGGGGAGG | |
| CTGAAGAGGCTGGGAATCACCCACATTCTGAATGCTGCGCATGGCACCGGCGTTTACACT | |
| GGCCCCGAATTCTACACTGGCCTGGAGATCCAGTACCTGGGTGTAGAGGTGGATGACTTT | |
| CCTGAGGTCGACATTTCCCAGCATTTCCGGAAGGCCTCTGAGTTCCTGGATGAGGCGCTG | |
| CTGACTTACACAGGGAAAGTCCTGGTCAGCAGCGAAATGGGCATCAGCCGGTCAGCAGTG | |
| CTGGTGGTCGCCTACCTGATGATCTTCCACAACATGGCCATCCTGGAGGCTTTGATGACC | |
| GTGCGTAAGAAGCGGGCCATCTACCCCAATGAGGGCTTCCTGAAGCAGCTGCGGGAGCTC | |
| AATGAGAAGTTGATGGAGCAGAGAGAAGAGGACTATGGCCGGGAGGGGGGATCAGCTCAG | |
| GCTGAGGAGGGCGAGGGCACTGGGAGCATGCTCGGGGCCAGAGTGCACGCCCTGACGGTG | |
| GAAGAGGAGGACGACAGCGCCAGCCACCTGAGTGCCTCCTCCCTGGGGAAGGCCACCCAG | |
| GCCTCCAAGCCCCTCACCCTCATAGACGAGGAGGAGGAGGAGAAACTGTACGAGCAGTGG | |
| AAGAAGGGGCAGGGCCTCCTCTCAGACAAGGTCCCCCAGGATGGAGGTGGCTGGCGCTCA | |
| GCCTCCTCTGGCCAGGGTGGGGAGGAGCTCGAGGACGAGGACGTGGAGAGGATCATCCAG | |
| GAGTGGCAGAGCCGAAACGAGAGGTACCAAGCAGAAGGGTACCGGAGGTGGGGAAGGGAG | |
| GAGGAGAAGGAGGAGGAGAGCGACGCTGGCTCCTCGGTGGGGAGGCGGCGGCGCACCCTG | |
| AGCGAGAGCAGCGCCTGGGAGAGCGTGAGCAGCCACGACATCTGGGTCCTGAAGCAGCAG | |
| CTGGAGCTGAACCGCCCGGACCACGGCAGGAGGCGCCGCGCAGACTCGATGTCCTCGGAG | |
| AGCACCTGGGACGCATGGAACGAGAGGCTGCTGGAGATTGAGAAGGAGGCTTCCCGGAGG | |
| TACCACGCCAAGAGCAAGAGAGAGGAGGCGGCAGACAGGAGCTCAGAAGCAGGGAGCAGG | |
| GTGCGGGAGGATGATGAGGACAGCGTGGGCTCTGAGGCCAGTTCCTTCTACAACTTCTGC | |
| AGCAGGAACAAGGACAAGCTCACTGCCCTGGAAAGATGGAAGATCAAGAGAATCCAATTT | |
| GGATTTCACAAGAAAGACTTGGGAGCGGGAGACAGCAGCGGTGAGCCCGGTGCAGAGGAG | |
| GCAGTAGGGGAGAAGAACCCCTCCGACGTCAGCCTGACAGCCTACCAGGCCTGGAAGCTG | |
| AAACACCAGAAGAAGGTGGGCAGTGAGAACAAGGAGGAGGTGGTGGAGCTCAGCAAGGGG | |
| GAGGACTCGGCCTTGGCTAAGAAGAGACAACGGAGGCTGGAGCTGCTGGAGAGAAGCCGG | |
| CAGACGCTGGAGGAGAGCCAGTCTATGGCAAGCTGCGAGGCGGACAGCTCCACGGCCAGC | |
| GGGAGCATTCCCCTGTCTGCGTTCTGGTCTGCAGACCCCTCAGTCAGCGCTGATGGGGAC | |
| ACGACGTCAGTACTGAGCACCCAGAGCCACCGCTCCCACCTGTCTCAGGCTGCAAGCAAC | |
| ATAGCGGGGTGTTCAACCTCCAACCCCACCACACCCCTGCCTAACCTGCCAGTGGGGCCT | |
| GGAGACACCATTTCCATTGCCAGTATCCAGAACTGGATTGCCAATGTAGTCAGTGAGACC | |
| CTTGCTCAGAAGCAAAATGAAATGCTGCTGTTGTCCCGCTCACCGTCTGTTGCAAGCATG | |
| AAGGCAGTACCAGCGGCTAGCTGCCTGGGGGATGACCAAGTCTCCATGCTTAGTGGACAC | |
| AGCAGCTCCTCCTTGGGTGGCTGCCTGTTGCCTCAGAGCCAGGCAAGACCCAGCTCTGAC | |
| ATGCAGTCTGTGCTGTCCTGCAACACCACACTGAGCTCACCCGCGGAAAGTTGCAGAAGC | |
| AAAGTGAGGGGGACCAGCAAGCCCATCTTCAGCCTCTTTGCTGACAATGTGGACCTAAAG | |
| GAACTTGGCCCGAAGGAGAAGGAGATGCAGATGGAGCTTAGGGAGAAGATGTCTGAGTAC | |
| AAAATGGAAAAGCTGGCCTCAGACAACAAACGCAGCTCCCTCTTCAAGAAGAAGAAGGTC | |
| AAGGAAGATGAGGATGATGGTGTGGGTGATGGGGATGAGGACACTGACAGTGCCATAGGG | |
| AGCTTCCGATATTCTTCCCGCAGTAATTCCCAGAAACCTGAAACAGACACATGCTCCTCC | |
| CTGGCTGTCTGTGATCACTATGCAAGTGGCAGCAGAGTTGGCAAAGAGATGGATAGCAGT | |
| ATTAATAAGTGGCTCAGTGGCCTCAGGACGGAGGAAAAACCTCCTTTCCAAAGTGACTGG | |
| TCTGGAAGTTCCAGAGGGAAGTACACCAGATCGTCCCTGCTCAGGGAGACAGAGTCTAAA | |
| TCCTCCAGTTACAAGTTTTCCAAATCCCAGTCAGAGGAACAGGACACCTCCTCCTACCAC | |
| GAGGCAAATGGCAACTCTGTAAGAAGCACTTCACGGTTCTCATCTTCCTCCACCAGGGAG | |
| GGCAGAGAGATGCACAAGTTCTCCAGGTCCACGTACAACGAGACCTCAAGTTCCCGAGAG | |
| GAGAGCCCAGAGCCCTACTTCTTCCGCCGGACCCCAGAGTCCTCAGAAAGGGAAGAGTCC | |
| CCAGAACCACAGCGCCCAAATTGGGCCAGGTCCAGGGACTGGGAAGATGTGGAAGAGTCA | |
| TCCAAGTCAGACTTCTCTGAATTTGGAGCCAAGAGGAAGTTCACCCAGAGCTTTATGAGG | |
| TCTGAAGAAGAGGGAGAGAAAGAGAGGACAGAAAACAGAGAAGAAGGGAGGTTTGCATCT | |
| GGACGGCGGTCCCAGTATCGGAGAAGCACTGACAGGGAGGAAGAGGAAGAAATGGACGAT | |
| GAAGCCATCATTGCTGCTTGGAGACGCCGGCAAGAAGAAACCAGGACCAAGCTGCAGAAA | |
| AGGAGGGAGGACTGAGCTGGGGAAAATCTGAG | |

The NOV65b polypeptide (SEQ ID NO:230) encoded by SEQ ID NO:229 is 1064 amino acids in length and is presented using the one-letter amino acid code in Table 65D. The Psort profile for NOV65b predicts that this sequence is a Type II membrane protein, and is likely to be localized at the plasma membrane with a certainty of 0.7900. In alternative embodiments, a NOV65b polypeptide is located to Goligi bodies with a certainty of 0.3000 or to the nucleus with a certainty of 0.8200.

TABLE 65D
NOV65b Polypeptide Sequence

| | |
|---|---|
| MLPKRVREKMDDTSLYNTPCVLDLQRALVQDRQEAPWNEVDEVWPNVFIAEKSVAVNKGR | (SEQ ID NO:230) |
| LKRLGITHILNAAHGTGVYTGPEFYTCLEIQYLGVEVDDFPEVDISQHFRKASEFLDEAL | |
| LTYRGKVLVSSEMGISRSAVLVVAYLMIFHNMAILEALMTVRKKRAIYPNEGFLKQLREL | |
| NEKLMEEREEDYGREGGSAEAEEGEGTGSMLGARVHALTVEEEDDSASHLSGSSLGKATQ | |
| ASKPLTLIDEEEEEKLYEQWKKGQGLLSDKVPQDGGGWRSASSGQGGEELEDEDVERIIQ | |
| EWQSRNERYQAEGYRRWGREEEKEEESDAGSSVGRRRRTLSESSAWESVSSHDIWVLKQQ | |
| LELNRPDHGRRRRADSMSSESTWDAWNERLLEIEKEASRRYHAKSKREEAADRSSEAGSR | |
| VREDDEDSVGSEASSFYNFCSRNKDKLTALERWKIKRIQFGFHKKDLGAGDSSGEPGAEE | |
| AVGEKNPSDVSLTAYQAWKLKHQKKVGSENKEEVVELSKGEDSALAKKRQRRLELLERSR | |
| QTLEESQSMASWEADSSTASGSIPLSAFWSADPSVSADGDTTSVLSTQSHRSHLSQAASN | |
| IAGCSTSNPTTPLPNLPVGPGDTISIASIQNWIANVVSETLAQKQNEMLLLSRSPSVASM | |
| KAVPAASCLGDDQVSMLSGHSSSSLGGCLLPQSQARPSSDMQSVLSCNTTLSSPAESCRS | |
| KVRGTSKPIFSLFADNVDLKELGRKEKEMQMELREKMSEYKMEKLASDNKRSSLFKKKKV | |
| KEDEDDGVGDGDEDTDSAIGSFRYSSRSNSQKPETDTCSSLAVCDHYASGSRVGKEMDSS | |
| INKWLSGLRTEEKPPFQSDWSGSSRGKYTRSSLLRETESKSSSYKFSKSQSEEQDTSSYH | |
| EANGNSVRSTSRFSSSSTREGREMHRFSRSTYNETSSSREESPEPYFFRRTPESSEREES | |
| PEPQRPNWARSRDWEDVEESSKSDFSEFGAKRKFTQSFMRSEEEGEKERTENREEGRFAS | |
| GRRSQYRRSTDREEEEEMDDEAIIAAWRRRQEETRTKLQKRRED | |

A BLAST analysis of NOV65 was run against the proprietary PatP GENESEQ Protein Patent database. It was found, for example, that the amino acid sequence of NOV65 had high homology to other proteins as shown in Table 65E.

TABLE 65E
BLASTX results from PatP database for NOV65

| | | Smallest |
|---|---|---|
| | | Sum |
| | High | Probability |
| Sequences producing High-scoring Segment Pairs: | Score | P(N) |
| patp: AAE04836 Human SGP018 phosphatase | 858 | 1.5e−85 |
| polypeptide | | |
| patp: AAB40919 Human ORFX ORF683 | 767 | 6.5e−76 |
| polypeptide sequence | | |
| patp: AAE04837 Human SGP003 phosphatase | 410 | 4.4e−38 |
| polypeptide | | |
| patp: AAY68779 Amino acid sequence of a human | 389 | 7.4e−36 |
| patp: AAB42334 Human ORFX ORF2098 | 389 | 7.4e−36 |
| polypeptide sequence | | |

In a search of sequence databases, it was found, for example, that the nucleic acid sequence of this invention has 328 of 531 bases (61%) identical to a gb:GENBANK-D:AB027004|acc:AB027004.1 mRNA from Homo sapiens (mRNA for protein phosphatase). The full amino acid sequence of the protein of the invention was found to have 80 of 174 amino acid residues (45%) identical to, and 115 of 174 amino acid residues (66%) similar to, the 198 amino acid residue ptnr:SPTREMBL-ACC:Q9UII6 protein from Homo sapiens (Human) (PROTEIN PHOSPHATASE). NOV65 also has homology to the other proteins shown in the BLASTP data in Table 65F.

TABLE 65F
NOV65 BLASTP results

| Gene Index/ | | Length | Identity | Positive | |
|---|---|---|---|---|---|
| Identifier | Protein/Organism | (aa) | (%) | (%) | Expect |
| gi\|14602535\|gb\| | protein phosphatase | 198 | 81/178 | 117/178 | 3e−37 |
| AAH09778.1\| | [Homo sapiens] | | (45) | (65) | |
| AAH09778 | | | | | |
| (BC009778) | | | | | |
| gi\|17454087\|ref\| | similar to protein | 370 | 82/186 | 121/186 | 4e−37 |
| XP_061191.1\| | phosphatase (H. sapiens) | | (44) | (64) | |
| (XM_061191) | [Homo sapiens] | | | | |
| gi\|7705959\|ref\| | protein phosphatase | 198 | 81/178 | 116/178 | 1e−36 |
| NP_057448.1\| | [Homo sapiens] | | (45) | (64) | |
| (NM_016364) | | | | | |
| gi\|7305011\|ref\| | dual specificity | 198 | 80/178 | 115/178 | 3e−36 |
| NP_038877.1\| | phosphatase 13 | | (44) | (63) | |
| (NM_013849) | [Mus musculus] | | | | |
| gi\|12839241\|dbj\| | dual specificity | 198 | 79/178 | 114/178 | 3e−35 |
| BAB24480.1\| | phosphatase 13˜putative | | (44) | (63) | |
| (AK006247) | [Mus musculus] | | | | |

This BLASTP data is displayed graphically in the ClustalW in Table 65G. A multiple sequence alignment is given, with the NOV65a and b proteins being shown on lines 1 and 2 in a ClustalW analysis comparing the protein of the invention with the related protein sequences shown in Table 65F.

[image]

[image]

[image]

[image]

[image]

Table 65H lists the domain description from DOMAIN analysis results against NOV65. This indicates that the NOV65 sequence has properties similar to those of other proteins known to contain this domain.

TABLE 65H
Domain Analysis of NOV65

gnl|Pfam|pfam00782, DSPc, Dual specificity phosphatase, catalytic domain.
Ser/Thr and Tyr protein phoaphatases. The enzyme's tertiary fold is highly
similar to that of tyrosine-specific phosphatases, except for a “recognition”
region. SEQ ID NO:864
CD-Length = 139 residues, 97.8% aligned
Score = 113 bits (282), Expect = 1e−26

| | | | |
|---|---|---|---|
| NOV65: | 42 | EVWPNVFIAEKSVAVNKGRLKRLGITHILNAAHGTGVYTGPEFYTGLEIQYLGVEVDDFP | 101 |
| | | E+ P++++ A N L +LGITH++N F YL + VDD | |
| Sbjct: 4 | EILPHLYLGSYPTASNLAFLSKLGITHVINVTEEVPNSKNSGF------LYLHIPVDDNH | 57 | |
| NOV65: | 102 | EVDISQHFRKASEFLDEALLTYRGKVLVSSEMGISRSAVLVVAYLMIFHNMAILEALMTV | 161 |
| | | E DIS + +A EF+++A GKVLV + GISRSA L++AYLM N+++ EA V | |
| Sbjct: | 58 | ETDISPYLDEAVEFIEDAR-QKGGKVLVHCQAGISRSATLIIAYLMKTRNLSLNEAYSFV | 116 |
| NOV65: | 162 | RKKR-AIYPNEGFLKQLRELNEK 183 | |
| | | +++R I PN GF +QL E K | |
| Sbjct: | 117 | KERRPIISPNFGFKRQLIEYERK 139 | |

The NOV65 gene of invention is a member of the family of dual specificity protein phosphatases (DSPs; Martell et al., Mol Cells Feb. 28, 1998;8(1):2-11). DSPs recognize either serine/threonine (Ser/Thr) or tyrosine (Tyr) moieties as targets for dephosphorylation. These enzymes regulate mitogenic signal transduction and can thereby regulate the cell cycle. Some members of this family are effective tumor suppressors, for example, PTEN. PTEN is required during embryonic development and later in life, and mutations in this gene give rise to different kinds of inherited and sporadic cancers (Eng, Recent Prog Horm Res 1999;54:441-52; discussion 453). In Drosophila, members of the DSP family, such as puckered, have important roles in development (Martin-Blanco et al., Genes Dev Feb. 15, 1998;12(4):557-70). The crystal structure of one member of the DSP family has been elucidated (Yuvaniyama at al., Science May 31, 1996;272(5266):1328-31) and this family has been successfully targeted for small molecule drug development (Ducruet et al., Bioorg Med Chem June 2000;8(6):1451-66). In addition, overexpression of a DSP has been demonstrated to be a potential therapy for cardiac hypertrophy (Bueno et al., Circ Res Jan. 19, 2001;88(1):88-96). NOV65 has closest homology to a phosphatase that is differentially regulated in the testis during spermatogenesis and is therefore thought to be involved in sperm development and maturation (Nakamura et al., Biochem. J. 344 Pt 3, 819-825 (1999)).

The disclosed NOV65 is predicted to be expressed in at least the following tissues: heart, skeletal muscle, colon, fetal lung, head, and ovary. This information was derived by determining the tissue sources of the sequences that were included in the invention including but not limited to SeqCalling sources, public EST sources, literature sources, and/or RACE sources. Further expression data for NOV65 is provided in Example 2.

The NOV65 nucleic acids and proteins of are useful in potential therapeutic applications implicated in various pathological disorders described further herein, for example, cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, transplantation, fertility, polycystic ovarian syndrome, cancer, tissue degeneration, bacterial/viral/parasitic infection, systemic lupus erythematosus, autoimmune disease, asthma, emphysema, scleroderma, allergy, ARDS, muscular dystrophy, Lesch-Nyhan syndrome, myasthenia gravis, Hirschsprung's disease, Crohn's Disease, appendicitis as well as other diseases, disorders and conditions. The NOV65 nucleic acid encoding the phosphatase-like protein of the invention, or fragments thereof, may further be useful in diagnostic applications, wherein the presence or amount of the nucleic acid or the protein are to be assessed. The novel nucleic acid of the invention encoding a protein phosphatase-like protein includes the nucleic acid whose sequence is provided in Table 65A or 65C, or a fragment thereof. The invention also includes a mutant or variant nucleic acid any of whose bases may be changed from the corresponding base shown in Table 65A or 65C while still encoding a protein that maintains its protein phosphatase-like activities and physiological functions, or a fragment of such a nucleic acid.

The invention further includes nucleic acids whose sequences are complementary to the sequence indicated in Table 65A or 65C, including nucleic acid fragments that are complementary to any of the nucleic acids just described. The invention additionally includes nucleic acids or nucleic acid fragments, or complements thereto, whose structures include chemical modifications. Such modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 39% of the bases may be so changed.

The novel protein of the invention includes the protein phosphatase-like protein whose sequence is provided in Table 65B or 65D. The invention also includes a mutant or variant protein any of whose residues may be changed from the corresponding residue shown in Table 65B or 65D while still encoding a protein that maintains its protein phosphatase-like activities and physiological functions, or a functional fragment thereof. In the mutant or variant protein, up to about 54% of the amino acid residues may be so changed.

These materials are further useful in the generation of antibodies that bind immunospecifically to the substances of the invention for use in therapeutic or diagnostic methods. These antibodies may be generated according to methods known in the art, using prediction from hydrophobicity charts, as described in the “Anti-NOVX Antibodies” section below.

NOV66

The disclosed NOV66 (alternatively referred to herein as CG56633-01) includes the 1036 nucleotide sequence (SEQ ID NO:231) shown in Table 66A. A NOV66 ORF begins with a Kozak consensus ATG initiation codon at nucleotides 28-30 and ends with a stop codon at nucleotides 913-915. The disclosed NOV66 maps to human chromosome 3.

TABLE 66A
NOV66 Nucleotide Sequence

| | |
|---|---|
| CCCGTCCTCGCTGGGTTGTCCCGGTCCATGTATCTGGTCATGGTGCTGAGGAACCTGCTC | (SEQ ID NO:231) |
| ATCATCCTGGCTGTCAGCTCTGACTCCCACCTCCACACCCCCATGTACTTCTTCCTCTCC | |
| AACCTGTGCTGGGCTGACATCGGTTTCACCTCGGCCATGGTTCCCAAGATGATTGTGGAC | |
| ATGCAGTCTCATAGCAGAGTCATCTCTTATGCGGGCTGCCTGACACGGATGTCTTTCTTG | |
| GTCCTTTTTGCATGTATAGAAGACATGCTCCTGACTGCGATGGCCTATGACTGCTTTGTA | |
| GCCATCTGTCGCCCTCTGCACTACCCAGTCATCGTGAATCCTCACCTCTCTGTCTTCTTA | |
| GTTTTGGTGTCCTTTTTCCTTAGCCTGTTGGATTCCCAGCTGCACAGTTTGATTGTGTTA | |
| CAATTCACCTTCTTCAAGAATGTGGAAATCTCTAATTTTGTCTGTGAGCCATCTCAGCTT | |
| CTCAACCTTGCCTGTTCTGACAGCGTCATCAATACCATATTCTTATATTTCGATAGTACT | |
| ATGTTTGGTTTTCTTCCCATTTCAAGGATCCTTTTGTCTTACTATAAAATTGTCCCCTCC | |
| ATTCTAAGGATTTCATCGTCAGATGGGAAGTATAAAGCCTTCTCCACCTGTGGCTCTCAC | |
| CTAGCAGTTGTTTGCTTATTTTATGGAACAGGCATTGGCGTGTACCTGACTTCAGCTGTG | |
| TCACCACCCCCCAGCAGTGGTGTGGTGGCGTCAGTGATGTACGCTCTGGTCACCCCCATG | |
| CTGAACCCTTTCATCTATAGCCTGAGAAACAGAGACATTCAAAGCGCCCTCTGGAGGCTG | |
| CGCAGCAGAACAGTCGAATCTCATGATCTGTTCCATCCTTTTTCTTGTGTGGGTAAGAAA | |
| GGGCAACCACATTAAATCCCTGCATCTGCAAATCCTGCTCCTTAGTCACATTATTTTTGT | |
| GGCTTGATGGCTTTTATTCCTTTCCGCATTTCCTATGTGAATATTGTTTTCTTCGTTATG | |
| CCTTTAACTGGAATGG | |

A NOV66 polypeptide (SEQ ID NO:232) encoded by SEQ ID NO:231 is 295 amino acids in length and is presented using the one-letter amino acid code in Table 66B. The Psort profile for NOV66 predicts that this sequence is a Type IIIa membrane protein, has a signal peptide and is likely to be localized at the plasma membrane with a certainty of 0.6400. In alternative embodiments, a NOV66 polypeptide is located to Golgi bodies with a certainty of 0.4600, or to the endoplasmic reticulum (membrane) with a certainty of 0.3700. The Signal P predicts a likely cleavage site for a NOV66 peptide is between positions 17 and 18, i.e., at the dash in the sequence AVS-SD.

TABLE 66B
NOV66 Polypeptide Sequence

| | |
|---|---|
| MYLVMVLRNLLIILAVSSDSHLHTPMYFFLSNLCWADIGFTSAMVPKMIVDMQSHSRVIS | (SEQ ID NO:232) |
| YAGCLTRMSFLVLFACIEDMLLTANAYDCFVATCRPLHYPVIVNPHLSVFLVLVSFFLSL | |
| LDSQLHSLIVLQFTFFKNVEISNFVCEPSQLLNLACSDSVINSIFLYFDSTMFGFLPISR | |
| ILLSYYKIVPSILRISSSDGKYKAFSTCGSHLAVVCLFYGTGIGVYLTSAVSPPPRSGVV | |
| ASVMYAVVTPMLNPFIYSLRNRDIQSALWRLRSRTVESHDLFHPFSCVGKKGQPH | |

A BLAST analysis of NOV66 was run against the proprietary PatP GENESEQ Protein Patent database. It was found, for example, that the amino acid sequence of NOV66 had high homology to other proteins as shown in Table 66C.

TABLE 66C
BLASTX results from PatP database for NOV66

| | | Smallest |
|---|---|---|
| | | Sum |
| | High | Probability |
| Sequences producing High-scoring Segment Pairs: | Score | P(N) |
| patp: AAG71875 Human olfactory receptor | 1466 | 5.6e−150 |
| polypeptide | | |
| patp: AAE04583 Human G-protein coupled | 1377 | 1.5e−140 |
| receptor-39 | | |
| patp: AAU24551 Human olfactory receptor | 1377 | 1.5e−140 |
| AOLFR38 | | |
| patp: AAG71816 Human olfactory receptor | 1363 | 4.6e−139 |
| polypeptide | | |
| patp: AAU24549 Human olfactory receptor | 1354 | 4.1e−138 |
| AOLFR36 | | |

In a search of sequence databases, it was found, for example, that the nucleic acid sequence of this invention has 973 of 1036 bases (93%) identical to a gb:GENBANK-ID:AF042089|acc:AF042089.1 mRNA from Homo sapiens (chromosome 3, olfactory receptor pseudogene cluster 1, complete sequence, and myosin light chain kinase (MLCK) pseudogene, partial sequence). The full amino acid sequence of the protein of the invention was found to have 192 of 265 amino acid residues (72%) identical to, and 221 of 265 amino acid residues (83%) similar to, the 264 amino acid residue ptnr:SPTREMBL-ACC:043789 protein from Homo sapiens (Human) (OLFACTORY RECEPTOR). NOV66 also has homology to the other proteins shown in the BLASTP data in Table 66D.

TABLE 66D
NOV66 BLASTP results

| Gene Index/ | | Length | Identity | Positive | |
|---|---|---|---|---|---|
| Identifier | Protein/Organism | (aa) | (%) | (%) | Expect |
| gi\|17466082\|ref\| | similar to olfactory | 349 | 221/250 | 229/250 | e−111 |
| XP_070192.1\| | receptor, family 7, | | (88) | (91) | |
| (XM_070192) | subfamily C, member 3 | | | | |
| | (H. sapiens) | | | | |
| | [Homo sapiens] | | | | |
| gi\|17482057\|ref\| | similar to G protein- | 251 | 220/250 | 229/250 | e−105 |
| XP_064778.1\| | coupled receptor homolog | | (88) | (91) | |
| (XM_064778) | clone G3 (H. sapiens) | | | | |
| | [Homo sapiens] | | | | |
| gi\|17448458\|ref\| | similar to OLFACTORY | 528 | 221/242 | 221/242 | e−101 |
| XP_070402.1\| | RECEPTOR 7C2 (OLFACTORY | | (87) | (91) | |
| (XM_070402) | RECEPTOR 19-18) (OR19- | | | | |
| | 18) (H. sapiens) [Homo | | | | |
| | sapiens] | | | | |
| gi\|7443955\|pir\| | olfactory receptor, HT2 - | 264 | 192/265 | 221/265 | 6e−92 |
| \|PC4369 | human (fragment) | | (72) | (82) | |
| gi\|4092819\|gb\| | BC319430_5 | 263 | 191/264 | 220/264 | 44−91 |
| AAD03353.1\| | [Homo sapiens] | | (72) | (82) | |

This BLASTP data is displayed graphically in the ClustalW in Table 66E. A multiple sequence alignment is given, with the NOV66 protein being shown on line 1 in a ClustalW analysis comparing the protein of the invention with the related protein sequences shown in Table 66D.

[image]

[image]

[image]

Table 66F lists the domain description from DOMAIN analysis results against NOV66. This indicates that the NOV66 sequence has properties similar to those of other proteins known to contain this domain.

TABLE 66F
Domain Analysis of NOV66

gnl|Pfam|pfam00001, 7tm_1, 7 transmembrane receptor
(rhodopsin family) SEQ ID NO:810
CD-Length = 254 residues, 996% aligned
Score = 88.6 bits (218), Expect = 5e−19

| | | | |
|---|---|---|---|
| NOV66: | 9 | NLLIILAVSSDSHLHTPMYFFLSNLCWADIGFTSAMVPKMIVDMQSHSRVISYAGCLTRM | 68 |
| | | NLL+IL + L TP FL NL AD+ F + P + + V A C | |
| Sbjct: 2 | NLLVILVILRTKKLRTPTNIFLLNLAVADLLFLLTLPPWALYYLVGGDWVFGDALCKLVG | 61 | |
| NOV66: | 69 | SFLVLFACIEDMLLTAMAYDCFVAICRPLHYPVIVNPHLSVFLVLVSFFLSLLDSQLHSL | 128 |
| | | + v+ +LLTA++ D ++AI PL Y I P + L+L+ + L+LL S L | |
| Sbjct: | 62 | ALFVVNGYASILLLTAISIDRYLAIVHPLRYRRIRTPRRAKVLILLVWVLALLLSLPPLL | 121 |
| NOV66: | 129 | IVLQFTFFKNVEISNFVCEPSQLLNLACSDSVINSIFLYFDSTMFGFLPISRILLSYYRI | 188 |
| | | T + + P + + ++ + + LP+ IL+ Y +I | |
| Sbjct: | 122 | FSWLRTVEEGNTTVCLIDFPEESV---------KRSYVLLSTLYGFVLPLLVILVCYTRI | 172 |
| NOV66: 189 | VP---------SILRISSSDGKYKAFSTCGSHLAVVCLFYGTGIGVYL----TSAVSPPP | 235 | |
| | | + L+ SS + A + V + I + L ++ | |
| Sbjct: | 173 | LRTLRKRARSQRSLKRRSSSERKAAKMLLVVVVVFVLCWLPYHIVLLLDSLCLLSIWRVL | 232 |
| NOV66: | 236 | RSGVVASVMYAVVTPMLNPFIY 257 | |
| | | + ++ ++ A V LNP IY | |
| Sbjct: | 233 | FTALLITLWLAYVNSCLNPIIY 254 | |

The olfactory system is able to distinguish several thousand odorant molecules. Olfactory receptors are believed to be encoded by an extremely large subfamily of G protein-coupled receptors. These receptors share a 7-transmembrane domain structure with many neurotransmitter and hormone receptors. They are responsible for the recognition and G protein-mediated transduction of odorant signals. The genes encoding these receptors are devoid of introns within their coding regions. Schurmans et al. (1993) cloned a member of this family of genes, OLFR1, from a genomic library by cross-hybridization with a gene fragment obtained by PCR. By isotopic in situ hybridization, they mapped the gene to 17p13-p12 with a peak at band 17p13. A minor peak was detected on chromosome 3, with a maximum in the region 3q13-q21. After MspI digestion, a RFLP was demonstrated. Using this in a study of 3 CEPH pedigrees, they demonstrated linkage with D17S126 at 17pter-p12; maximum lod=3.6 at theta=0.0. Used as a probe on Southern blots under moderately stringent conditions, the cDNA hybridized to at least 3 closely related genes. Ben-Arie et al. (1994) cloned 16 human OLFR genes, all from 17p13.3. The intronless coding regions are mapped to a 350-kb contiguous cluster, with an average intergenic separation of 15 kb. The OLFR genes in the cluster belong to 4 different gene subfamilies, displaying as much sequence variability as any randomly selected group of OLFRs. This suggested that the cluster may be one of several copies of an ancestral OLFR gene repertoire whose existence may have predated the divergence of mammals. Localization to 17p13.3 was performed by fluorescence in situ hybridization as well as by somatic cell hybrid mapping.

The ability to distinguish different odors depends on a large number of different odorant receptors (ORs). Sullivan et al. (1996) noted that ORs are expressed by nasal olfactory sensory neurons; each neuron expresses only 1 allele of a single OR gene. In the nose, different sets of ORs are expressed in distinct spatial zones. Neurons that express the same OR gene are located in the same zone; however, in that zone they are randomly interspersed with neurons expressing other ORs. This distribution suggested to the authors that, when the cell chooses an OR gene for expression, it may be restricted to a specific zonal gene set, but it may select from that set by a stochastic mechanism. Proposed models of OR gene choice fall into 2 classes: locus-dependent and locus-independent. Locus-dependent models posit that OR genes are clustered in the genome, perhaps with members of different zonal gene sets clustered at distinct loci. In contrast, locus-independent models do not require that OR genes be clustered. To assess the feasibility of these models, Sullivan et al. (1996) determined the expression zones, sequences, and chromosomal locations of a number of mouse OR genes. They mapped OR genes to 11 different regions on 7 chromosomes. These loci lie within paralogous chromosomal regions that appear to have arisen by duplications of large chromosomal domains followed by extensive gene duplication and divergence. These studies showed that OR genes expressed in the same zone map to numerous loci. Moreover, a single locus can contain genes expressed in different zones. These findings raised the possibility that OR gene choice is locus-independent or involved consecutive stochastic choices.

Nekrasova et al. (1996) overexpressed human (OR17-4) and rat (olp4) olfactory receptor genes in insect cells, purified them, and characterized them biochemically. They identified monomeric, dimeric, and trimeric forms of the proteins corresponding to molecular weights of 32, 69, and 94 kD by electrophoresis. The oligomers were resistant to reduction and alkylation and were therefore thought to be held together by SDS-resistant hydrophobic interactions, consistent with observations of other G protein-coupled receptors.

Glusman et al. (1996) described the results of complete sequencing of an OR-rich cosmid spanning the center of the OR gene cluster on 17p13.3. The resulting 40-kb sequence revealed 3 known OR coding regions, 2 OR genes which may have originated from a tandem duplication event, and a new OR pseudogene fused to another OR gene.

Issel-Tarver and Rine (1996) characterized 4 members of the canine olfactory receptor gene family. The 4 subfamilies comprised genes expressed exclusively in olfactory epithelium. Analysis of large DNA fragments using Southern blots of pulsed field gels indicated that subfamily members were clustered together, and that 2 of the subfamilies were closely linked in the dog genome. Analysis of the 4 olfactory receptor gene subfamilies in 26 breeds of dog provided evidence that the number of genes per subfamily was stable in spite of differential selection on the basis of olfactory acuity in scent hounds, sight hounds, and toy breeds.

Issel-Tarver and Rine (1997) performed a comparative study of 4 subfamilies of olfactory receptor genes first identified in the dog to assess changes in the gene family during mammalian evolution, and to begin linking the dog genetic map to that of humans. These 4 families were designated by them OLF1, OLF2, OLF3, and OLF4 in the canine genome. The subfamilies represented by these 4 genes range in size from 2 to 20 genes. They are all expressed in canine olfactory epithelium but were not detectably expressed in canine lung, liver, ovary, spleen, testis, or tongue. The OLF1and OLF2 subfamilies are tightly linked in the dog genome and also in the human genome. The smallest family is represented by the canine OLF1gene. Using dog gene probes individually to hybridize to Southern blots of genomic DNA from 24 somatic cell hybrid lines. They showed that the human homologous OLF1 subfamily maps to human chromosome 11. The human gene with the strongest similarity to the canine OLF2 gene also mapped to chromosome 11. Both members of the human subfamily that hybridized to canine OLF3 were located on chromosome 7. It was difficult to determine to which chromosome or chromosomes the human genes that hybridized to the canine OLF4 probe mapped. This subfamily is large in mouse and hamster as well as human, so the rodent background largely obscured the human cross-hybridizing bands. It was possible, however, to discern some human-specific bands in blots corresponding to human chromosome 19. They refined the mapping of the human OLF1 homolog by hybridization to YACs that map to 11q11. In dogs, the OLF1 and OLF2 subfamilies are within 45 kb of one another (Issel-Tarver and Rine (1996)). Issel-Tarver and Rine (1997) demonstrated that in the human OLF1 and OLF2 homologs are likewise closely linked. By studying YACs, Issel-Tarver and Rine (1997) found that the human OLF3 homolog maps to 7q35. A chromosome 19-specific cosmid library was screened by hybridization with the canine OLF4 gene probe, and clones that hybridized strongly to the probe even at high stringency were localized to 19p13.1 and 19p13.2. These clones accounted, however, for a small fraction of the homologous human bands.

Rouquier et al. (1998) demonstrated that members of the olfactory receptor gene family are distributed on all but a few human chromosomes. Through fluorescence in situ hybridization analysis, they showed that OR sequences reside at more than 25 locations in the human genome. Their distribution was biased for terminal bands of chromosome arms. Flow-sorted chromosomes were used to isolate 87 OR sequences derived from 16 chromosomes. Their sequence relationships indicated the inter- and intrachromosomal duplications responsible for OR family expansion. Rouquier et al. (1998) determined that the human genome has accumulated a striking number of dysfunctional copies: 72% of these sequences were found to be pseudogenes. ORF-containing sequences predominate on chromosomes 7, 16, and 17.

Trask et al. (1998) characterized a subtelomeric DNA duplication that provided insight into the variability, complexity, and evolutionary history of that unusual region of the human genome, the telomere. Using a DNA segment cloned from chromosome 19, they demonstrated that the blocks of DNA sequence shared by different chromosomes can be very large and highly similar. Three chromosomes appeared to have contained the sequence before humans migrated around the world. In contrast to its multicopy distribution in humans, this subtelomeric block maps predominantly to a single locus in chimpanzee and gorilla, that site being nonorthologous to any of the locations in the human genome. Three new members of the olfactory receptor (OR) gene family were found to be duplicated within this large segment of DNA, which was found to be present at 3q, 15q, and 19p in each of 45 unrelated humans sampled from various populations. From its sequence, one of the OR genes in this duplicated block appeared to be potentially functional. The findings raised the possibility that functional diversity in the OR family is generated in part through duplications and interchromosomal rearrangements of the DNA near human telomeres.

Mombaerts (1999) reviewed the molecular biology of the odorant receptor (OR) genes in vertebrates. Buck and Axel (1991) discovered this large family of genes encoding putative odorant receptor genes. Zhao et al. (1998) provided functional proof that one OR gene encodes a receptor for odorants. The isolation of OR genes from the rat by Buck and Axel (1991) was based on 3 assumptions. First, ORs are likely G protein-coupled receptors, which characteristically are 7-transmembrane proteins. Second, ORs are likely members of a multigene family of considerable size, because an immense number of chemicals with vastly different structures can be detected and discriminated by the vertebrate olfactory system. Third, ORs are likely expressed selectively in olfactory sensory neurons. Ben-Arie et al. (1994) focused attention on a cluster of human OR genes on 17p, to which the first human OR gene, OR1D2, had been mapped by Schurmans et al. (1993). According to Mombaerts (1999), the sequences of more than 150 human OR clones had been reported. The human OR genes differ markedly from their counterparts in other species by their high frequency of pseudogenes, except the testicular OR genes. Research showed that individual olfactory sensory neurons express a small subset of the OR repertoire. In rat and mouse, axons of neurons expressing the same OR converge onto defined glomeruli in the olfactory bulb.

Gilad et al. (2000) reported the population sequence diversity of genomic segments within a 450-kb cluster of olfactory receptor (OR) genes on chromosome 17. They found a dichotomy in the pattern of nucleotide diversity between OR pseudogenes and introns on the one hand and the closely interspersed intact genes on the other. They suggested that weak positive selection is responsible for the observed patterns of genetic variation. This was inferred from a lower ratio of polymorphism to divergence in genes compared with pseudogenes or introns, high nonsynonymous substitution rates in OR genes, and a small but significant overall reduction in variability in the entire OR gene cluster compared with other genomic regions. The dichotomy among functionally distinct segments within a short genomic distance requires high recombination rates within this OR cluster.

NOV66 is predicted to be expressed in at least the following tissues: lung, liver, ovary, spleen, testis. This information was derived by determining the tissue sources of the sequences that were included in the invention including but not limited to SeqCalling sources, public EST sources, literature sources, and/or RACE sources. Further expression data for NOV66 is provided in Example 2.

The nucleic acids and proteins of NOV66 are useful in potential therapeutic applications implicated in various pathological disorders described further herein, for example, cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, transplantation, fertility, polycystic ovarian syndrome, cancer, tissue degeneration, bacterial/viral/parasitic infection, systemic lupus erythematosus, autoimmune disease, asthma, emphysema, scleroderma, allergy, ARDS, muscular dystrophy, Lesch-Nyhan syndrome, myasthenia gravis, Hirschsprung's disease, Crohn's Disease, appendicitis as well as other diseases, disorders and conditions. The NOV66 nucleic acid encoding the GPCR-like protein of the invention, or fragments thereof, may further be useful in diagnostic applications, wherein the presence or amount of the nucleic acid or the protein are to be assessed. A NOV66 nucleic acid of the invention encoding a Olfactory receptor-like protein includes the nucleic acid whose sequence is provided in Table 66A, or a fragment thereof. The invention also includes a mutant or variant nucleic acid any of whose bases may be changed from the corresponding base shown in Table 66A while still encoding a protein that maintains its Olfactory receptor-like activities and physiological functions, or a fragment of such a nucleic acid.

The invention further includes nucleic acids whose sequences are complementary to the sequence disclosed in Table 66A, including nucleic acid fragments that are complementary to any of the nucleic acids just described. The invention additionally includes nucleic acids or nucleic acid fragments, or complements thereto, whose structures include chemical modifications. Such modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 7% of the bases may be so changed.

The novel protein of the invention includes the olfactory receptor-like protein whose sequence is provided in Table 66B. The invention also includes a mutant or variant protein any of whose residues may be changed from the corresponding residue shown in Table 66B while still encoding a protein that maintains its Olfactory receptor-like activities and physiological functions, or a functional fragment thereof. In the mutant or variant protein, up to about 28% of the amino acid residues may be so changed.

These materials are further useful in the generation of antibodies that bind immunospecifically to the substances of the invention for use in therapeutic or diagnostic methods. These antibodies may be generated according to methods known in the art, using prediction from hydrophobicity charts, as described in the “Anti-NOVX Antibodies” section below.

NOV67

The disclosed NOV67 (alternatively referred to herein as CG56571-01) includes the 1072 nucleotide sequence (SEQ ID NO:233) shown in Table 67A. A NOV67 ORF begins with a Kozak consensus ATG initiation codon at nucleotides 41-43 and ends with a stop codon at nucleotides 989-991. The disclosed NOV67 maps to human chromosome 7.

TABLE 67A
NOV67 Nucleotide Sequence

| | |
|---|---|
| CATTCCCTCCTAACCACCTAGATTGAAGAAGTGAGGTTCAATGTCCCAACTGGGAAGGGA | (SEQ ID NO:233) |
| CAACATAACCTGGGTGAGTGAGTTCATCCTAATGGGTCTCTCCAGTGACAGGCAGACCCA | |
| GGCTGGACTCTTTATCTTATTTGGGGCTGCCTACCTGCTGACCCTGCTGGGCAATGGGCT | |
| CATCCTGCTCCTGATCTGGCTGGACGTGAGACTCCACCTGCCCATGTATTTCTTCCTCTG | |
| CAACCTCTCACTTGTCGACATCTGCTACACCTCCAGCAGGGTCCCTCAGATGCTGGTGCA | |
| CTGCACCAGCAAAAGAAAGACCATCTCCTTTGCCCGATGTGGGACCCAGCTCTTTTTCTC | |
| CCTGGCCCTCGGAGGGACCGAGTTTTTGTTGCTGGCCGCAATGGCCTATGACCGCTACGT | |
| GGCTGTTTGCGACCCCCTGTGTTACATAGCAGTGATGAGCCCAAGGCTCTGCATGGCACT | |
| GGCAGCTGTCTCTTGGCTAGTGGGCCTGGCTAATTCTGCTATGGAAACGGCACTGACCAT | |
| GCACCTGCCCACCTGTGGGCACAACGTGCTGAACCATGTGGCCTGTGAGACACTGGCACT | |
| GGTCACGTCGGCCTGCGTGGACATCACCTTCAATCAGGTGGTCATAGTGGCCTCCAGTGT | |
| GGTGGTGCTGCTGGTGCCCTGCTGCCTGGTCTCGCTGTCCTACACCCTCATTGTAGTTGC | |
| CGTCCTGCAGATCCACTCCACCCAGGGGCACCGCAAGGCCTTTGGGACCTGTGCCTCCCA | |
| CCTCACTGTGGTCTCCATATCCTATGGGATGGCCCTCTTTACCTACATGCAGCCTCGCTC | |
| CATGGCCTCAGCTGAGCAGGAAAAGGTGATGGTACTCTCTTATGCTGTGGTGACCCCCAT | |
| GTTGAATCCTTTCATCTACAGTCTGCGGAACAAGGATGTGAAGGCAGCTCTGAGTCGAGC | |
| TCTGATGAGGAGCTCTGAATTAAAACATTAGAGAGTGGTTTGAGTAACAAGAAGGCCTCA | |
| CTCTGAAAACAGTGGGCATTGGACTGTGCTCTCCAGTATAACGTGTGTACGC | |

A NOV67 polypeptide (SEQ ID NO:234) encoded by SEQ ID NO:233 is 312 amino acids in length and is presented using the one-letter amino acid code in Table 67B. The Psort profile for NOV67 predicts that this sequence is a Type IIIb membrane protein, has a signal peptide, and is likely to be localized at the plasma membrane with a certainty of 0.6000. In alternative embodiments, a NOV67 polypeptide is located to Golgi bodies with a certainty of 0.4000, to the endoplasmic reticulum (membrane) with a certainty of 0.3000, or to the mitrochondrial membrane with a certainty of 0.3522. The Signal P predicts a likely cleavage site for a NOV67 peptide is between positions 58 and 59, i.e., at the dash in the sequence VRL-HL.

TABLE 67B
NOV67 Polypeptide Sequence

| | |
|---|---|
| MSQLGRDNITWVSEFILMGLSSDRQTQAGLFILFGAAYLLTLLGNGLILLLIWLDVRLHL | (SEQ ID NO:234) |
| PMYFFLCNLSLVDICYTSSRVPQMLVHCTSKRKTISFARCGTQLFFSLALGGTEFLLLAA | |
| MAYDRYVAVCDPLCYIAVMSPRLCMALAAVSWLVGLANSAMETALTMHLPTCGHNVLNHV | |
| ACETLALVRSACVDITFNQVVIVASSVVVLLVPCCLVSLSYTLIVVAVLQIHSTQGHRKA | |
| FGTCASHLTVVSISYGMALFTYMQPRSMASAEQEKVMVLSYAVVTPMLNPFIYSLRNKDV | |
| KAALSRALMRSSELKH | |

A BLAST analysis of NOV67 was run against the proprietary PatP GENESEQ Protein Patent database. It was found, for example, that the amino acid sequence of NOV67 had high homology to other proteins as shown in Table 67C.

TABLE 67C
BLASTX results from PatP database for NOV67

| | | Smallest |
|---|---|---|
| | | Sum |
| | High | Probability |
| Sequences producing High-scoring Segment Pairs: | Score | P(N) |
| patp: AAG71514 Human olfactory receptor | 1576 | 1.2e−161 |
| polypeptide | | |
| patp: AAG72330 Human OR-like polypeptide | 924 | 1.5e−92 |
| query sequence | | |
| patp: AAG72925 Human olfactory receptor data | 924 | 1.5e−92 |
| exploratorium | | |
| patp: AAG72977 Olfactory receptor-like | 924 | 1.5e−92 |
| polypeptide | | |
| patp: AAG71408 Human olfactory receptor | 923 | 1.9e−92 |
| polypeptide | | |

In a search of sequence databases, it was found, for example, that the nucleic acid sequence of this invention has 536 of 649 bases (82%) identical to a gb:GENBANK-ID:AF073974|acc:AF073974.1 mRNA from Mus musculus domesticus (Mus musculus domesticus clone OR28M olfactory receptor gene). The full amino acid sequence of the protein of the invention was found to have 179 of 311 amino acid residues (57%) identical to, and 228 of 311 amino acid residues (73%) similar to, the 317 amino acid residue ptnr:SWISSNEW-ACC:Q13607 protein from Homo sapiens (Human) (OLFACTORY RECEPTOR 2F1 (OLFACTORY RECEPTOR-LIKE PROTEIN OLF3)). NOV67 also has homology to the other proteins shown in the BLASTP data in Table 67D.

TABLE 67D
NOV67 BLASTP results

| Gene Index/ | | Length | Identity | Positive | |
|---|---|---|---|---|---|
| Identifier | Protein/Organism | (aa) | (%) | (%) | Expect |
| gi\|9297120\|sp\| | OLFACTORY RECEPTOR 2F1 | 317 | 179/311 | 228/311 | 7e−86 |
| Q13607\|O2F1_{—} | (OLFACTORY RECEPTOR-LIKE | | (57) | (72) | |
| HUMAN | PROTEIN OLF3) | | | | |
| gi\|6912558\|ref\| | olfactory receptor, | 317 | 179/311 | 228/311 | 9e−86 |
| NP_036501.1\| | family 2, subfamily F, | | (57) | (72) | |
| (NM_012369) | member 1; olfactory | | | | |
| | receptor, family 2, | | | | |
| | subfamily F, member 5; | | | | |
| | olfactory receptor, | | | | |
| | family 2, subfamily F, | | | | |
| | member 4 [Homo sapiens] | | | | |
| gi\|2495055\|sp\| | OLFACTORY RECEPTOR-LIKE | 317 | 176/305 | 228/305 | 2e−85 |
| Q95156\|OLF3_{—} | PROTEIN OLF3 | | (57) | (72) | |
| CANFA | | | | | |
| gi\|14423778\|sp\| | OLFACTORY RECEPTOR 2F2 | 317 | 175/308 | 220/308 | 7e−82 |
| O95006\|O2F2_{—} | (OLFACTORY RECEPTOR 7-1) | | (56) | (70) | |
| HUMAN | (OR7-1) (PID: g2495051) | | | | |
| | [Homo sapiens] | | | | |
| gi\|5453066\|gb\| | olfactory receptor | 216 | 167/216 | 189/216 | 3e−78 |
| AAD43423.1\| | [Mus musculus] | | (77) | (87) | |
| (AF073974) | | | | | |

This BLASTP data is displayed graphically in the ClustalW in Table 67E. A multiple sequence alignment is given, with the NOV67 protein being shown on line 1 in a ClustalW analysis comparing the protein of the invention with the related protein sequences shown in Table 67D.

[image]

Table 67F lists the domain description from DOMAIN analysis results against NOV67. This indicates that the NOV67 sequence has properties similar to those of other proteins known to contain this domain.

TABLE 67F
Domain Analysis of NOV67

gnl|Pfam|pfam00001, 7tm_1, 7 transmembrane receptor
(rhodopsin family). SEQ ID NO:810
CD-Length = 254 residues, 93.3% aligned
Score = 76.3 bits (186), Expect 3e−15

| | | | |
|---|---|---|---|
| NOV67: | 61 | PMYFFLCNLSLVDICYTSSRVPQMLVHCTSKRKTISFARCGTQLFFSLALGGTEFLLLAA | 120 |
| | | P FL NL++ D+ + + P L + A C + G LLL A | |
| Sbjct: | 18 | PTNIFLLNLAVADLLFLLTLPPWALYYLVGGDWVFGDALCKLVGALFVVNGYASILLLTA | 77 |
| NOV67: | 121 | MAYDRYVAVCDPLCYIAVMSPRLCMALAAVSWLVGLANSAMEVALTMHLPTCGHNVLNHV | 180 |
| | | ++ DRY+A+ PL Y + +PR L + W++ L S + N + | |
| Sbjct: | 78 | ISIDRYLAIVHPLRYRRIRTPRRAKVLILLVWVLALLLSLPPLLFSWLRTVEEGNTTVCL | 137 |
| NOV67: | 181 | ACETLALVRSACVDITFNQVVIVASSVVVLLVPCCLVSLSYTLIVVAVLQIHSTQGHRKA | 240 |
| | | V+ + V ++ ++ V+++ L +L L+ S+ + A | |
| Sbjct: | 138 | IDFPEESVKRSYVLLSTLVGFVLPLLVILVCYTRILRTLRKRARSQRSLKRRSSSERKAA | 197 |
| NOV67: | 241 | FGTCASHLTVVSISYG----MALFTYMQPRSMASAEQEKVMVLSYAVVTPMLNPFIY | 293 |
| | | + V + L + ++ L A V LNP IY | |
| Sbjct: | 198 | KMLLVVVVVFVLCWLPYHIVLLLDSLCLLSIWRVLPTALLITLWLAYVNSCLNPIIY | 254 |

The olfactory system is able to distinguish several thousand odorant molecules. Olfactory receptors are believed to be encoded by an extremely large subfamily of G protein-coupled receptors. These receptors share a 7-transmembrane domain structure with many neurotransmitter and hormone receptors. They are responsible for the recognition and G protein-mediated transduction of odorant signals. The genes encoding these receptors are devoid of introns within their coding regions. Schurmans et al. (1993) cloned a member of this family of genes, OLFR1, from a genomic library by cross-hybridization with a gene fragment obtained by PCR. By isotopic in situ hybridization, they mapped the gene to 17p13-p12 with a peak at band 17p13. A minor peak was detected on chromosome 3, with a maximum in the region 3q13-q21. After MspI digestion, a RFLP was demonstrated. Using this in a study of 3 CEPH pedigrees, they demonstrated linkage with D17S126 at 17pter-p12; maximum lod=3.6 at theta=0.0. Used as a probe on Southern blots under moderately stringent conditions, the cDNA hybridized to at least 3 closely related genes. Ben-Arie et al. (1994) cloned 16 human OLFR genes, all from 17p13.3. The intronless coding regions are mapped to a 350-kb contiguous cluster, with an average intergenic separation of 15 kb. The OLFR genes in the cluster belong to 4 different gene subfamilies, displaying as much sequence variability as any randomly selected group of OLFRs. This suggested that the cluster may be one of several copies of an ancestral OLFR gene repertoire whose existence may have predated the divergence of mammals. Localization to 17p13.3 was performed by fluorescence in situ hybridization as well as by somatic cell hybrid mapping.

The ability to distinguish different odors depends on a large number of different odorant receptors (ORs). Sullivan et al. (1996) noted that ORs are expressed by nasal olfactory sensory neurons; each neuron expresses only 1 allele of a single OR gene. In the nose, different sets of ORs are expressed in distinct spatial zones. Neurons that express the same OR gene are located in the same zone; however, in that zone they are randomly interspersed with neurons expressing other ORs. This distribution suggested to the authors that, when the cell chooses an OR gene for expression, it may be restricted to a specific zonal gene set, but it may select from that set by a stochastic mechanism. Proposed models of OR gene choice fall into 2 classes: locus-dependent and locus-independent. Locus-dependent models posit that OR genes are clustered in the genome, perhaps with members of different zonal gene sets clustered at distinct loci. In contrast, locus-independent models do not require that OR genes be clustered. To assess the feasibility of these models, Sullivan et al. (1996) determined the expression zones, sequences, and chromosomal locations of a number of mouse OR genes. They mapped OR genes to 11 different regions on 7 chromosomes. These loci lie within paralogous chromosomal regions that appear to have arisen by duplications of large chromosomal domains followed by extensive gene duplication and divergence. These studies showed that OR genes expressed in the same zone map to numerous loci. Moreover, a single locus can contain genes expressed in different zones. These findings raised the possibility that OR gene choice is locus-independent or involved consecutive stochastic choices.

Nekrasova et al. (1996) overexpressed human (OR17-4) and rat (olp4) olfactory receptor genes in insect cells, purified them, and characterized them biochemically. They identified monomeric, dimeric, and trimeric forms of the proteins corresponding to molecular weights of 32, 69, and 94 kD by electrophoresis. The oligomers were resistant to reduction and alkylation and were therefore thought to be held together by SDS-resistant hydrophobic interactions, consistent with observations of other G protein-coupled receptors.

Glusman et al. (1996) described the results of complete sequencing of an OR-rich cosmid spanning the center of the OR gene cluster on 17p13.3. The resulting 40-kb sequence revealed 3 known OR coding regions, 2 OR genes which may have originated from a tandem duplication event, and a new OR pseudogene fused to another OR gene.

Issel-Tarver and Rine (1996) characterized 4 members of the canine olfactory receptor gene family. The 4 subfamilies comprised genes expressed exclusively in olfactory epithelium. Analysis of large DNA fragments using Southern blots of pulsed field gels indicated that subfamily members were clustered together, and that 2 of the subfamilies were closely linked in the dog genome. Analysis of the 4 olfactory receptor gene subfamilies in 26 breeds of dog provided evidence that the number of genes per subfamily was stable in spite of differential selection on the basis of olfactory acuity in scent hounds, sight hounds, and toy breeds.

Issel-Tarver and Rine (1997) performed a comparative study of 4 subfamilies of olfactory receptor genes first identified in the dog to assess changes in the gene family during mammalian evolution, and to begin linking the dog genetic map to that of humans. These 4 families were designated by them OLF1, OLF2, OLF3, and OLF4 in the canine genome. The subfamilies represented by these 4 genes range in size from 2 to 20 genes. They are all expressed in canine olfactory epithelium but were not detectably expressed in canine lung, liver, ovary, spleen, testis, or tongue. The OLF1 and OLF2 subfamilies are tightly linked in the dog genome and also in the human genome. The smallest family is represented by the canine OLF1 gene. Using dog gene probes individually to hybridize to Southern blots of genomic DNA from 24 somatic cell hybrid lines. They showed that the human homologous OLF1 subfamily maps to human chromosome 11. The human gene with the strongest similarity to the canine OLF2 gene also mapped to chromosome 11. Both members of the human subfamily that hybridized to canine OLF3 were located on chromosome 7. It was difficult to determine to which chromosome or chromosomes the human genes that hybridized to the canine OLF4 probe mapped. This subfamily is large in mouse and hamster as well as human, so the rodent background largely obscured the human cross-hybridizing bands. It was possible, however, to discern some human-specific bands in blots corresponding to human chromosome 19. They refined the mapping of the human OLF1 homolog by hybridization to YACs that map to 11q11. In dogs, the OLF1 and OLF2 subfamilies are within 45 kb of one another (Issel-Tarver and Rine (1996)). Issel-Tarver and Rine (1997) demonstrated that in the human OLF1 and OLF2 homologs are likewise closely linked. By studying YACs, Issel-Tarver and Rine (1997) found that the human OLF3 homolog maps to 7q35. A chromosome 19-specific cosmid library was screened by hybridization with the canine OLF4 gene probe, and clones that hybridized strongly to the probe even at high stringency were localized to 19p13.1 and 19p13.2. These clones accounted, however, for a small fraction of the homologous human bands.

Rouquier et al. (1998) demonstrated that members of the olfactory receptor gene family are distributed on all but a few human chromosomes. Through fluorescence in situ hybridization analysis, they showed that OR sequences reside at more than 25 locations in the human genome. Their distribution was biased for terminal bands of chromosome arms. Flow-sorted chromosomes were used to isolate 87 OR sequences derived from 16 chromosomes. Their sequence relationships indicated the inter- and intrachromosomal duplications responsible for OR family expansion. Rouquier et al. (1 998) determined that the human genome has accumulated a striking number of dysfunctional copies: 72% of these sequences were found to be pseudogenes. ORF-containing sequences predominate on chromosomes 7, 16, and 17.

Trask et al. (1998) characterized a subtelomeric DNA duplication that provided insight into the variability, complexity, and evolutionary history of that unusual region of the human genome, the telomere. Using a DNA segment cloned from chromosome 19, they demonstrated that the blocks of DNA sequence shared by different chromosomes can be very large and highly similar. Three chromosomes appeared to have contained the sequence before humans migrated around the world. In contrast to its multicopy distribution in humans, this subtelomeric block maps predominantly to a single locus in chimpanzee and gorilla, that site being nonorthologous to any of the locations in the human genome. Three new members of the olfactory receptor (OR) gene family were found to be duplicated within this large segment of DNA, which was found to be present at 3q, 15q, and 19p in each of 45 unrelated humans sampled from various populations. From its sequence, one of the OR genes in this duplicated block appeared to be potentially functional. The findings raised the possibility that functional diversity in the OR family is generated in part through duplications and interchromosomal rearrangements of the DNA near human telomeres.

Mombaerts (1999) reviewed the molecular biology of the odorant receptor (OR) genes in vertebrates. Buck and Axel (1991) discovered this large family of genes encoding putative odorant receptor genes. Zhao et al. (1998) provided functional proof that one OR gene encodes a receptor for odorants. The isolation of OR genes from the rat by Buck and Axel (1991) was based on 3 assumptions. First, ORs are likely G protein-coupled receptors, which characteristically are 7-transmembrane proteins. Second, ORs are likely members of a multigene family of considerable size, because an immense number of chemicals with vastly different structures can be detected and discriminated by the vertebrate olfactory system. Third, ORs are likely expressed selectively in olfactory sensory neurons. Ben-Arie et al. (1994) focused attention on a cluster of human OR genes on 17p, to which the first human OR gene, OR1D2, had been mapped by Schurmans et al. (1993). According to Mombaerts (1999), the sequences of more than 150 human OR clones had been reported. The human OR genes differ markedly from their counterparts in other species by their high frequency of pseudogenes, except the testicular OR genes. Research showed that individual olfactory sensory neurons express a small subset of the OR repertoire. In rat and mouse, axons of neurons expressing the same OR converge onto defined glomeruli in the olfactory bulb.

Gilad et al. (2000) reported the population sequence diversity of genomic segments within a 450-kb cluster of olfactory receptor (OR) genes on chromosome 17. They found a dichotomy in the pattern of nucleotide diversity between OR pseudogenes and introns on the one hand and the closely interspersed intact genes on the other. They suggested that weak positive selection is responsible for the observed patterns of genetic variation. This was inferred from a lower ratio of polymorphism to divergence in genes compared with pseudogenes or introns, high nonsynonymous substitution rates in OR genes, and a small but significant overall reduction in variability in the entire OR gene cluster compared with other genomic regions. The dichotomy among functionally distinct segments within a short genomic distance requires high recombination rates within this OR cluster.

NOV67 is predicted to be expressed in at least the following tissues: brain, testis, ovary, skeletal muscle, neuronal tissue. This information was derived by determining the tissue sources of the sequences that were included in the invention including but not limited to SeqCalling sources, public EST sources, literature sources, and/or RACE sources. Further expression data for NOV67 is provided in Example 2.

The NOV67 nucleic acids and proteins of are useful in potential therapeutic applications implicated in various pathological disorders described further herein. The NOV67 nucleic acid encoding the GPCR-like protein of the invention, or fragments thereof, may further be useful in diagnostic applications, wherein the presence or amount of the nucleic acid or the protein are to be assessed. The novel nucleic acid of the invention encoding an olfactory receptor-like protein OLF3-like protein includes the nucleic acid whose sequence is provided in Table 67A, or a fragment thereof.

The invention also includes a mutant or variant nucleic acid any of whose bases may be changed from the corresponding base shown in Table 67A while still encoding a protein that maintains its olfactory receptor-like protein OLF3-like activities and physiological functions, or a fragment of such a nucleic acid. The invention further includes nucleic acids whose sequences are complementary to the sequence disclosed in Table 67A, including nucleic acid fragments that are complementary to any of the nucleic acids just described. The invention additionally includes nucleic acids or nucleic acid fragments, or complements thereto, whose structures include chemical modifications. Such modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 18% of the bases may be so changed.

The novel protein of the invention includes the olfactory receptor-like protein OLF3-like protein whose sequence is provided in Table 67B. The invention also includes a mutant or variant protein any of whose residues may be changed from the corresponding residue shown in Table 67B while still encoding a protein that maintains its olfactory receptor-like protein OLF3-like activities and physiological functions, or a functional fragment thereof. In the mutant or variant protein, up to about 43% of the amino acid residues may be so changed.

These materials are further useful in the generation of antibodies that bind immunospecifically to the substances of the invention for use in therapeutic or diagnostic methods. These antibodies may be generated according to methods known in the art, using prediction from hydrophobicity charts, as described in the “Anti-NOVX Antibodies” section below.

NOV68

The disclosed NOV68 (alternatively referred to herein as CG56844-01) includes the 2580 nucleotide sequence (SEQ ID NO:235) shown in Table 68A. A NOV68 ORF begins with a Kozak consensus ATG initiation codon at nucleotides 21-23 and ends with a TAG codon at nucleotides 1896-1898. The disclosed NOV68 maps to human chromosome 9.

TABLE 68A
NOV68 Nucleotide Sequence

| | |
|---|---|
| CAGGCCCCCACGTGGACAGCATGGACCGCGGCACGCTCCCTCTGGCTGTTGCCCTGCTGC | (SEQ ID NO:235) |
| TGGCCAGCTGCAGCCTCAGCCCCACAAGTCTTGCAGAAACAGTCCATTGTGACCTTCAGC | |
| CTGTGGGCCCCGAGAGGGGCGAGGTGACATATACCACTAGCCAGGTCTCGAAGGGCTGCG | |
| TGGCTCAGGCCCCCAATGCCATCCTTGAAGTCCATGTCCTCTTCCTGGAGTTCCCAACGG | |
| GCCCGTCACAGCTGGACCTGACTCTCCAGGCATCCAAGCAAAATGGCACCTGGCCCCGAG | |
| AGGTGCTTCTGGTCCTCAGTGTAAACAGCAGTGTCTTCCTGCATCTCCAGGCCCTGGGAA | |
| TCCCACTGCACTTGGCCTACAATTCCAGCCTGGTCACCTTCCAAGAGCCCCCGGGGGTCA | |
| ACACCACAGAGCTGCCATCCTCCTCCTCCTCCTCACTGTCCTTCTGCATGCTGGAAGCCA | |
| GCCAGGACATGGGCCGCACGCTCGAGTGGCGGCCGCGTACTCCAGCCTTGGTCCCGGGCT | |
| GCCACTTGGAAGGCGTGGCCGGCCACAAGGAGGCGCACATCCTGAGGGTCCTGCCGGGCC | |
| ACTCGGCCGGGCCCCGGACGGTGACGGTGAAGGTGGAACTGAGCTGCGCACCCGGGGATC | |
| TCGATGCCGTCCTCATCCTGCAGGGTCCCCCCTACGTGTCCTGGCTCATCGACGCCAACC | |
| ACAACATGCAGATCTGGACCACTGGAGAATACTCCTTCAAGATCTTTCCAGAGAAAAACA | |
| TTCGTGGCTTCAAGCTCCCAGACACACCTCAAGGCCTCCTGGGGGAGGCCCGCATGCTCA | |
| ATGCCAGCATTGTGGCATCCTTCGTGGAGCTACCGCTGGCCAGCATTGTCTCACTTCATG | |
| CCTCCAGCTGCGGTGGTAGGCTGCAGACCTCACCCGCACCGATCCAGACCACTCCTCCCA | |
| AGGACACTTGTAGCCCGGAGCTGCTCATGTCCTTGATCCAGACAAAGTGTGCCGACGACG | |
| CCATGACCCTGGTACTAAAGAAAGAGCTTGTTGCGCATTTGAAGTGCACCATCACGGGCC | |
| TGACCTTCTGGGACCCCAGCTGTGAGGCAGAGGACAGGGGTGACAAGTTTGTCTTGCGCA | |
| GTGCTTACTCCAGCTGTGGCATGCAGGTGTCAGCAAGTATGATCAGCAATGAGGCGGTGG | |
| TCAATATCCTGTCGAGCTCATCACCACAGCGGAAAAAGGTGCACTGCCTCAACATGCACA | |
| GCCTCTCTTTCCAGCTGGGCCTCTACCTCAGCCCACACTTCCTCCAGGCCTCCAACACCA | |
| TCGAGCCGGCGCAGCAGAGCTTTGTGCAGGTCAGAGTGTCCCCATCCGTCTCCGAGTTCC | |
| TGCTCCAGTTAGACAGCTGCCACCTGGACTTGGGGCCTGAGGGAGGCACCGTGGAACTCA | |
| TCCAGGGCCGGCCGGCCAAGGGCAACTGTGTGAGCCTGCTGTCCCCAAGCCCCGAGGGTG | |
| ACCCGCGCTTCAGCTTCCTCCTCCACTTCTACACAGTACCCATACCCAAAACCGGCACCC | |
| TCAGCTGCACGGTAGCCCTGCGTCCCAAGACCGGGTCTCAAGACCAGGAAGTCCATAGGA | |
| CTGTCTTCATGCGCTTGAACATCATCAGCCCTGACCTGTCTGGTTGCACAAGCAAAGGCC | |
| TCGTCCTGCCCGCCGTGCTGGGCATCACCTTTGGTGCCTTCCTCATCGGGGCCCTGCTCA | |
| CTGCTGCACTCTGGTACATCTACTCGCACACCCGTTCCCCCAGCAAGCGGGAGCCCGTGG | |
| TGGCGGTGGCTGCCCCGGCCTCCTCGGAGAGCAGCAGCACCAACCACACCATCGGGAGCA | |
| CCCAGAGCACCCCCTGCTCCACCAGCAGCATGGCATAGCCCCGGCCCCCCGCGCTCGCCC | |
| AGCAGGAGAGACTGAGCAGCCGCCAGCTGGGAGCACTGGTGTGAACTCACCCTGGGAGCC | |
| AGTCCTCCACTCGACCCAGAATGGAGCCTGCTCTCCGCGCCTACCCTTCCCCCCTCCCTC | |
| TCAGAGGCCTGCTGCCAGTGCAGCCACTGGCTTGGAACACCTTGGGGTCCCTCCACCCCA | |
| CAGAACCTTCAACCCAGTGGGTCTGGGATATGGCTGCCCAGGAGACAGACCACTTGCCAC | |
| GCTGTTGTAAAAACCCAAGTCCCTGTCATTTGAACCTGGATCCAGCACTGGTGAACTGAG | |
| CTGGGCAGGAAGGCAGAACTTGAAACAGATTCAGGCCAGCCCAGCCAGGCCAACAGCACC | |
| TCCCCCCTGGGAAGAGAAGAGGGCCCAGCCCAGAGCCACCTGGATCTATCCCTGCGGCCT | |
| CCACACCTGAACTTGCCTAACTAACTGGCAGGGGAGACAGGAGCCTAGCGGAGCCCAGCC | |
| TGGGAGCCCAGAGGGTGGCAAGAACAGTGGGCGTTGGGAGCCTAGCTCCTGCCACATGGA | |
| GCCCCCTCTGCCGGTCGGGCAGCCAGCAGAGGGGGAGTAGCCAAGCTGCTTGTCCTGGGC | |
| CTGCCCCTGTGTATTCACCACCAATAAATCAGACCATGAAACCAGTGAAAAAAAAAAAAA | |

A NOV68 polypeptide (SEQ ID NO:236) encoded by SEQ ID NO:235 is 625 amino acids in length and is presented using the one-letter amino acid code in Table 68B. The Psort profile for NOV68 predicts that this sequence is a Type IIIa membrane protein, has a signal peptide, and is likely to be localized at the plasma membrane with a certainty of 0.6400. In alternative embodiments, a NOV68 polypeptide is located to Golgi bodies with a certainty of 0.4600, or to the endoplasmic reticulum (membrane) with a certainty of 0.3700. The Signal P predicts a likely cleavage site for a NOV68 peptide is between positions 25 and 26, i.e., at the dash in the sequence SLA-ET.

TABLE 68B
NOV68 Polypeptide Sequence

| | |
|---|---|
| MDRGTLPLAVALLLASCSLSPTSLAETVHCDLQPVGPERGEVTYTTSQVSKGCVAQAPNA | (SEQ ID NO:236) |
| ILEVHVLFLEFPTGPSQLELTLQASKQNGTWPREVLLVLSVNSSVFLHLQALGIPLHLAY | |
| NSSLVTFQEPPGVNTTELPSSSSSSLSFCMLEASQDMGRTLEWRPRTPALVRGCHLEGVA | |
| GHKEAHILRVLPGHSAGPRTVTVKVELSCAPGDLDAVLILQGPPYVSWLIDANHNMQIWT | |
| TGEYSFKIFPEKNIRGFKLPDTPQGLLGEARMLNASIVASFVELPLASIVSLHASSCGGR | |
| LQTSPAPIQTTPPKDTCSPELLMSLIQTKCADDAMTLVLKKELVAHLKCTITGLTFWDPS | |
| CEAEDRGDKFVLRSAYSSCGMQVSASMISNEAVVNILSSSSPQRKKVHCLNMDSLSFQLG | |
| LYLSPHFLQASNTIEPGQQSFVQVRVSPSVSEFLLQLDSCHLDLGPEGGTVELIQGRAAK | |
| GNCVSLLSPSPEGDPRFSFLLHFYTVPIPKTGTLSCTVALRPKTGSQDQEVHRTVFMRLN | |
| IISPDLSGCTSKGLVLPAVLGITFGAFLIGALLTAALWYIYSHTRSPSKREPVVAVAAPA | |
| SSESSSTNHSIGSTQSTPCSTSSMA | |

A BLAST analysis of NOV68 was run against the proprietary PatP GENESEQ Protein Patent database. It was found, for example, that the amino acid sequence of NOV68 had high homology to other proteins as shown in Table 68C.

TABLE 68C
BLASTX results from PatP database for NOV68

| | | Smallest |
|---|---|---|
| | | Sum |
| | High | Probability |
| Sequences producing High-scoring Segment Pairs: | Score | P(N) |
| patp: AAR54828 Endoglin - Homo sapiens, 658 aa. | 2500 | 1.5e−259 |
| patp: AAR99802 Endoglin - Homo sapiens, 658 aa. | 2500 | 1.5e−259 |
| patp: AAY82190 Human endoglin SEQ ID NO: 2 - | 2500 | 1.5e−259 |
| Homo sapiens | | |
| patp: AAR37808 Rat betaglycan - Synthetic, | 215 | 2.2e−24 |
| 853 aa. | | |
| patp: AAR74601 Rat betaglycan contg. transforming | 215 | 2.2e−24 |
| growth factor | | |

In a search of sequence databases, it was found, for example, that the nucleic acid sequence of this invention has 2128 of 2128 bases (100%) identical to a gb:GENBANK-ID:HUMENDO|acc:J05481.1 mRNA from Homo sapiens (Human endoglin mRNA, 3′ end). The full amino acid sequence of the protein of the invention was found to have 509 of 624 amino acid residues (81%) identical to, and 531 of 624 amino acid residues (85%) similar to, the 658 amino acid residue ptnr:SWISSNEW-ACC:P17813 protein from Homo sapiens (Human) (ENDOGLIN PRECURSOR (CD105 ANTIGEN)). NOV68 also has homology to the other proteins shown in the BLASTP data in Table 68D.

TABLE 68D
NOV68 BLASTP results

| Gene Index/ | | Length | Identity | Positive | |
|---|---|---|---|---|---|
| Identifier | Protein/Organism | (aa) | (%) | (%) | Expect |
| gi\|3041681\|sp\| | ENDOGLIN PRECURSOR | 658 | 621/658 | 632/658 | 0.0 |
| P17813\|EGLN_{—} | (CD105 ANTIGEN) | | (94) | (94) | |
| HUMAN | | | | | |
| gi\|15679936\|gb\| | endoglin (Osler-Rendu- | 658 | 620/658 | 621/658 | 0.0 |
| AAH14271.1\| | Weber syndrome 1) | | (94) | (94) | |
| AAH14271 | [Homo sapiens] | | | | |
| (BC014271) | | | | | |
| gi\|105920\|pir\| | endoglin precursor - | 645 | 607/644 | 608/644 | 0.0 |
| \|A36262 | human | | (94) | (94) | |
| gi\|4557555\|ref\| | endoglin precursor; | 625 | 581/618 | 582/618 | 0.0 |
| NP_000109.1\| | Endoglin | | (94) | (94) | |
| (NM_000118) | [Homo sapiens] | | | | |
| gi\|6679649\|ref\| | endoglin | 653 | 452/660 | 540/660 | 0.0 |
| NP_031958.1\| | [Mus musculus] | | (68) | (75) | |
| (NM_007932) | | | | | |

This BLASTP data is displayed graphically in the ClustalW in Table 68E. A multiple sequence alignment is given, with the NOV68 protein being shown on line 1 in a ClustalW analysis comparing the protein of the invention with the related protein sequences shown in Table 68D.

[image]

[image]

[image]

Table 68F lists the domain description from DOMAIN analysis results against NOV68. This indicates that the NOV68 sequence has properties similar to those of other proteins known to contain this domain.

TABLE 68F
Domain Analysis of NOV68

gnl|Smart|smart00241, ZP, Zona pellucida (ZP) domain; ZP proteins are
responsible for sperm-adhesion to the zona pellucida. ZP domains are also
present in multidomain transmembrane proteins such as glycoprotein GP2,
uromodulin and TOF-beta receptor type III (betaglycan). SEQ ID NO:1391
CD-Length = 253 residues, 96.4% aligned
Score = 42.4 bits (98), Expect = 8e−05

| | | | |
|---|---|---|---|
| NOV68: | 329 | KCADDAMTLVLKKELVAHLKCTITGLTFWDPSCEAEDRG---DKFVLRSAYSSCGMQVSA | 385 |
| | | +C +D M + + +L+ + GLT DPSC G + CG + | |
| Sbjct: | 1 | QCGEDRMVVSVSTDLLFPGGIYVKGLTLGDPSCRPVFVGANSAVVSFEVPLNECGTRRQV | 60 |
| NOV68: | 386 | S----MISNEAVVNILSSSSP---------------QRKKVHCLNMD------------- | 413 |
| | | + + SN VV+ + + L D | |
| Sbjct: | 61 | NPDGIVYSNTLVVSPIFHPLFITRDDRANYHVQCFYPESEKVSLRADVSTIPPTPLSVVS | 120 |
| NOV68: | 414 | --SLSFQLGLYLSPHF---LQASNTIEPGQQSFVQVRVSPSVSE-FLLQLDSCHLDLGP- | 466 |
| | | + LY F Q+++T++ G + + L + +C+ G | |
| Sbjct: | 121 | EGPPTCTYSLYKDDSFGSPYQSADTVQLGDPVYHEWSCDGRDDPSLGLLVHNCYATPGSD | 180 |
| NOV68: | 467 | --EGGTVELIQGRAAKGNCVSLLSPSPEGDPRFSFLLHFYTVPIPKTGTLSCTVALRPKT | 524 |
| | | G +I + +SP F + + C + L K+ | |
| Sbjct: | 181 | PFSGPKYFIIDNGCPVDRYLDSVSPYSSPSHYARFSVKVFKFADRSLVYFHCQITLCDKS | 240 |
| NOV68: | 525 | GSQD 528 | |
| Sbjct: | 241 | DGSS 244 | |

Transforming growth factor-beta (TGF-beta) plays an important role in angiogenesis and vascular function. Endoglin, a transmembrane TGF-beta binding protein, is highly expressed on vascular endothelial cells and is the target gene for the hereditary haemorrhagic telangiectasia type I (HHT1), a dominantly inherited vascular disorder. The specific function of endoglin responsible for HHT1 is believed to involve alterations in TGF-beta responses. The initial interactions on the cell surface between endoglin and TGF-beta receptors may be an important mechanism by which endoglin modulates TGF-beta signalling, and thereby responses. On human microvascular endothelial cells, endoglin is co-expressed and is associated with betaglycan, a TGF-beta accessory receptor with which endoglin shares limited amino acid homology. This complex formation may occur in either a ligand-dependent or a ligand-independent manner. In addition, three higher order complexes containing endoglin, type II and/or type I TGF-beta receptors, also can occur on these cells. Thus endoglin may modify TGF-beta signalling by interacting with both betaglycan and the TGF-beta signalling receptors at physiological receptor concentrations and ratios (Wong et al., 2000, Eur J Biochem vol. 267: 5550-60).

Endoglin is a homodimeric membrane glycoprotein. In association with transforming growth factor (TGF)-ss receptors I and II, endoglin can also bind TGF-ss1 and -ss3 and form a functional receptor complex. In human vascular tissue, endoglin immunolabeling is shown to be higher in endarterectomy specimens removed from diseased coronary arteries than in normal internal mammary arteries. In vitro, antisense oligonucleotides to endoglin is shown to decrease its expression and antagonized the TGF-ss-mediated inhibition of human and porcine SMC migration. Thus, upregulation of endoglin occurs during arterial repair and in established atherosclerotic plaques and may be required for modulation of SMC migration by TGF-ss (Ma X et al., 2000, Arterioscler Thromb Vasc Biol vol. 20 :2546-52).

Hereditary hemorrhagic telangiectasia (HHT) is an inherited autosomal dominant vascular dysplasia caused by mutations in either endoglin (HHT1) or activin-like kinase receptor-1 (ALK-1) (HHT2). The majority of the mutations in endoglin cause frameshifts and premature stop codons. Although initial reports suggested a dominant-negative model for HHT1, more recent reports have suggested that mutations in endoglin lead to haploinsufficiency. Expression of the missense mutants alone revealed that they are misfolded and that most show no cell surface expression. When co-expressed with wild-type endoglin, the missense mutants are able to dimerize with the normal endoglin protein and are trafficked to the cell surface. Thus either dominant-negative protein interactions or haploinsufficiency can cause HHT1 (Lux et al., 2000, Hum Mol Genet vol 9: 745-55).

NOV68 is predicted to be expressed in at least the following tissues: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea and uterus. This information was derived by determining the tissue sources of the sequences that were included in the invention including but not limited to SeqCalling sources, public EST sources, literature sources, and/or RACE sources. Further expression data for NOV68 is provided in Example 2.

The NOV68 nucleic acids and proteins of are useful in potential therapeutic applications implicated in various pathological disorders described further herein, for example, the compositions of the present invention will have efficacy for the treatment of patients suffering from: arterial injuries, cerebral arteriovenous malformalities, pregnancy complications, carcinomas such as breast and mammary carcinoma as well as other diseases, disorders and conditions. The NOV68 nucleic acid encoding the endoglin-like protein of the invention, or fragments thereof, may further be useful in diagnostic applications, wherein the presence or amount of the nucleic acid or the protein are to be assessed. The novel nucleic acid of the invention encoding a Endoglin (CD105 antigen)-like protein includes the nucleic acid whose sequence is provided in Table 68A, or a fragment thereof. The invention also includes a mutant or variant nucleic acid any of whose bases may be changed from the corresponding base shown in Table 68A while still encoding a protein that maintains its Endoglin (CD105 antigen)-like activities and physiological functions, or a fragment of such a nucleic acid. The invention further includes nucleic acids whose sequences are complementary to the sequence disclosed in Table 68A, including nucleic acid fragments that are complementary to any of the nucleic acids just described.

The invention additionally includes nucleic acids or nucleic acid fragments, or complements thereto, whose structures include chemical modifications. Such modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 0% of the bases may be so changed.

The novel protein of the invention includes the Endoglin (CD105 antigen)-like protein whose sequence is provided in Table 68B. The invention also includes a mutant or variant protein any of whose residues may be changed from the corresponding residue shown in Table 68B while still encoding a protein that maintains its Endoglin (CD105 antigen)-like activities and physiological functions, or a functional fragment thereof. In the mutant or variant protein, up to about 19% of the amino acid residues may be so changed.

These materials are further useful in the generation of antibodies that bind immunospecifically to the substances of the invention for use in therapeutic or diagnostic methods. These antibodies may be generated according to methods known in the art, using prediction from hydrophobicity charts, as described in the “Anti-NOVX Antibodies” section below.

NOV69

NOV69 includes two IL1-like proteins, designated herein as NOV69a and NOV69b. These are splice variants of sequence accession number CG56950-01.

NOV69a

The disclosed NOV69a (alternatively referred to herein as CG56950-01) includes the 414 nucleotide sequence (SEQ ID NO: ) shown in Table 69A. A NOV69a ORF begins with a Kozak consensus ATG initiation codon at nucleotides 100-102 and ends with a TGA codon at nucleotides 412-414. The disclosed NOV69a maps to human chromosome 7.

TABLE 69A
NOV69a Nucleotide Sequence

| | |
|---|---|
| ATGGAAAAAGCATTGAAAATTGACACACCTCAGCAGGGGAGCATTCAGGATATCAATCAT | (SEQ ID NO:237) |
| CGGGTGTGGGTTCTTCAGGACCAGACGCTCATAGCAGTCCCGAGGAAGGACCGTATGTCT | |
| CCAGTCACTATTGCCTTAATCTCATGCCGACATGTGGAGACCCTTGAGAAACACAGAGGG | |
| AACCCCACACTGCAGCTGAAGGAAAAGGATATAATGGATTTGTACAACCAACCCGAGCCT | |
| GTGAAGTCCTTTCTCTTCTACCACAGCCAGAGTGGCAGGAACTCCACCTTCGAGTCTGTG | |
| GCTTTCCCTGGCTGGTTCATCGCTGTCAGCTCTGAAGGAGGCTGTCCTCTCATCCTTACC | |
| CAAGAACTGGGGAAAGCCAACACTACTGACTTTGGGTTAACTATGCTGTTTTAA | |

A NOV69a polypeptide (SEQ ID NO:238) encoded by SEQ ID NO:237 is 137 amino acids in length and is presented using the one-letter amino acid code in Table 69B. The Psort profile for NOV69a predicts that this sequence has no signal peptide and is likely to be localized to the cytoplasm with a certainty of 0.4500. In alternative embodiments, a NOV69a polypeptide is located to lysosomes with a certainty of 0.1514, or to perioxisomal microbodies with a certainty of 0.2384.

TABLE 69B
NOV69a Polypeptide Sequence

| | |
|---|---|
| MEKALKIDTPQQGSIQDINHRVWVLQDQTLIAVPRKDRMSPVTIALISCRHVETLEKDRG | (SEQ ID NO:238) |
| NPTLQLKEKDIMDLYNQPEPVKSFLFYHSQSGRNSTFESVAFPGWFIAVSSEGGCPLILT | |
| QELGKANTTDFGLTMLF | |

NOV69b

The disclosed NOV69b (alternatively referred to herein as CG56136-02) includes the 411 nucleotide sequence (SEQ ID NO: ) shown in Table 69C. A SEC2 ORF begins with a Kozak consensus ATG initiation codon at nucleotides 1-3 and ends with a stop codon at nucleotides 409-411. The disclosed NOV69b maps to human chromosome 2q12-14.1.

TABLE 69C
NOV69b Nucleotide Sequence

| | |
|---|---|
| ATGGAAAAAGCATTGAAAGTTGACACACCTCAGCGGGGGAGCATTCAGGATATCAATCAT | (SEQ ID NO:239) |
| CGGGTGTGGGTTCTTCAGGACCAGACGCTCATAGCAGTCCCGAGGAAGGACCGTATGTCT | |
| CCAGTCACTATTGCCTTAATCTCATGCCGACATGTGGAGACCCTTGAGAAAGACAGAGGG | |
| AACCCCATCTACCTGGGCCTGAATGGACTCAATCTCTGCCTGATGTGTGTTCAAGTCGGG | |
| GACCAGCCCACACTGCAGATGAACCAGAGTGGCAGGAACTCCACCTTCGAGTCTGTGGCT | |
| TTCCCTGGCTGGTTGATCGCTGTCAGCTCTGAAGGAGGCTGTCCTCTCATCCTTACCCAA | |
| GAACTGGGGAAAGCCAACACTACTGACTTTGGGTTAACTATGCTGTTTTAA | |

A NOV69b polypeptide (SEQ ID NO:240) encoded by SEQ ID NO:239 is 136 amino acids in length and is presented using the one-letter amino acid code in Table 69D. The Psort profile for NOV69b predicts that this sequence has no signal peptide and is likely to be localized to the cytoplasm with a certainty of 0.6500. In alternative embodiments, a NOV69b polypeptide is located to lysosomes with a certainty of 0.2305.

TABLE 69D
NOV69b Polypeptide Sequence

| | |
|---|---|
| MEKALKVDTPQRGSIQDINHRVWVLQDQTLIAVPRKDRMSPVTIALISCRHVETLEKDRG | (SEQ ID NO:240) |
| NPIYLGLNGLNLCLMCVQVGDQPTLQMNQSGRNSTFESVAFPGWLIAVSSEGGCPLILTQ | |
| ELGKANTTDFGLTMLF | |

A BLAST analysis of NOV69 was run against the proprietary PatP GENESEQ Protein Patent database. It was found, for example, that the amino acid sequence of NOV69 had high homology to other proteins as shown in Table 69E.

TABLE 69E
BLASTX results from PatP database for NOV69

| | | Smallest |
|---|---|---|
| | | Sum |
| | High | Probability |
| Sequences producing High-scoring Segment Pairs: | Score | P(N) |
| patp: AAW86286 Rodent interleukin (IL)-1 | 263 | 1.1e−38 |
| epsilon polypeptide | | |
| patp: AAY24049 Amino acid sequence of a murine | 263 | 1.1e−38 |
| SPOIL-II | | |
| patp: AAE06662 Mouse interleukin-1epsilon | 263 | 1.1e−38 |
| (IL-1epsilon) | | |
| patp: AAY70217 Human Interleukin-1 epsilon | 414 | 1.7e−38 |
| protein | | |
| patp: AAY70218 Human Interleukin-1 epsilon | 414 | 1.7e−38 |

In a search of sequence databases, it was found, for example, that the nucleic acid sequence of this invention has 258 of 263 bases (98%) identical to a gb:GENBANK-ID:AF201831|acc:AF201831.1 mRNA from Homo sapiens (FIL1 epsilon mRNA). The full amino acid sequence of the protein of the invention was found to have 82 of 89 amino acid residues (92%) identical to, and 87 of 89 amino acid residues (97%) similar to, the 158 amino acid residue ptnr:SPTREMBL-ACC:Q9UHA7 protein from Homo sapiens (Human) (FIL1 EPSILON). NOV69 also has homology to the other proteins shown in the BLASTP data in Table 69F.

TABLE 69F
NOV69 BLASTP results

| Gene Index/ | | Length | Identity | Positive | |
|---|---|---|---|---|---|
| Identifier | Protein/Organism | (aa) | (%) | (%) | Expect |
| gi\|7657092\|ref\| | Interleukin-1 | 158 | 137/158 | 137/158 | 7e−73 |
| NP_055255.1\| | Superfamily 1 [Homo | | (86) | (86) | |
| (NM_014440) | sapiens] | | | | |
| gi\|9506601\|ref\| | interleukin 1 family, | 160 | 77/156 | 104/105 | 4e−38 |
| NP_062323.1\| | member 6 (epsilon) [Mus | | (49) | (66) | |
| (NM_019450) | musculus] | | | | |
| gi\|9665234\|ref\| | interleukin-1 homolog 1 | 169 | 76/147 | 97/147 | 2e−35 |
| NP_062564.1\| | [Homo sapiens] | | (51) | (65) | |
| (NM_019618) | | | | | |
| gi\|7657090\|ref\| | Interleukin-1 | 157 | 62/145 | 85/145 | 2e−27 |
| NP_055253.1\| | Superfamily □ [Homo | | (42) | (57) | |
| (NM_014438) | sapiens] | | | | |
| gi\|12844800\|dbj\| | homolog to FIL1 | 183 | 58/144 | 77/144 | 4e−23 |
| BAB26505.1\| | ETA˜putative | | (40) | (53) | |
| (AK009787) | [Mus musculus] | | | | |

This BLASTP data is displayed graphically in the ClustalW in Table 69G. A multiple sequence alignment is given, with the NOV69 protein being shown on line 1 in a ClustalW analysis comparing the protein of the invention with the related protein sequences shown in Table 69F.

[image]

Table 69H lists the domain description from DOMAIN analysis results against NOV69. This indicates that the NOV69 sequence has properties similar to those of other proteins known to contain this domain.

TABLE 69H
Domain Analysis of NOV69

gnl|Pfam|pfam00340, IL1, Interleukin-1/18. This family includes
interleukin-1 and interleukin-18. SEQ ID NO:1392
CD-Length = 142 residues
Score = 60.1 bits (144), Expect = 8e−11

| | | | |
|---|---|---|---|
| NOV69: | 57 | KDRGNPTLQLKEKDIMDLYNQPEPVKSFLFYHSQSGRNSTFESVAFPGWFIAVSSEGGCP | 116 |
| | | K+ P LQL+ + E K F F ++ G FES A+P WFIA E P | |
| Sbjct: | 62 | KEGDEPVLQLEMVEPPKYIKNSEMDKRFFFEKTEIGSKVYFESAAYPNFIATKQEEDRP | 121 |
| NOV69: | 117 | LILTQELGKANTTDF 131 | |
| | | + L +++TDF | 131 |
| Sbjct: | 122 | VFLANGPPESDITDF 136 | |

There are two structurally distinct forms of IL1: IL1(alpha), which is the acidic form with pI5, and IL1(beta) (IL1B; 147720), the neutral form with pI7. Both are 17-kD proteins coded by separate genes. The IL1A gene has 10,206 bp with 7 exons and 6 introns (Furutani et al., 1986). By Southern transfer analysis of DNAs from human-rodent somatic cell hybrids, Modi et al. (1988) assigned the IL1A gene to chromosome 2. Regional localization to 2q13-q21 was achieved by in situ hybridization. Lafage et al. (1989) confirmed assignment to 2q13 by in situ hybridization.

The IL1A and IL1B proteins, which are synthesized by a variety of cell types including activated macrophages, keratinocytes, stimulated B lymphocytes, and fibroblasts, are potent mediators of inflammation and immunity. Lord et al. (1991) demonstrated that both the alpha and beta forms, but particularly the beta form, are transcribed in polymorphonuclear leukocytes stimulated with LPS. Both IL1A and IL1B stimulate osteoclast activity in vitro and are potent bone resorbing factors. Sabatino et al. (1988) studied the effects of 72-hour subcutaneous infusions of interleukins 1-alpha and -beta on plasma, calcium, and bone morphology. Both interleukins 1 caused a marked, dose-dependent increase in plasma calcium. Increased numbers of osteoclasts and bone resorption surfaces were observed on quantitative histomorphometry of bone. The results suggest a role for IL1 in the modulation of extracellular fluid calcium homeostasis. Hogquist et al. (1991) demonstrated that interleukin-1 is involved in apoptosis (cell death). Both the alpha and the beta forms are released as a consequence of cell injury regardless of the insult.

Bailly et al. (1993) elucidated a polymorphism that consists of a variable number of repeats of a 46-bp sequence within intron 6 of the IL1A gene. Among 72 unrelated persons, they identified 6 different alleles ranging from 5 to 18 repeats; the most frequent allele, present in 62%, contained 9 repeats. They suggested that the polymorphism may be of significance in gene function, since each repeat contains 3 potential binding sites for transcription factors.

Gray et al. (1986) showed that in the mouse also there are at least 2 interleukin-1 genes, Il1(alpha) and Il1(beta). Boultwood et al. (1989) used in situ chromosome hybridization to show that the 2 Il1 genes in the mouse are located in the F region of chromosome 2. It had previously been shown by studies in mouse-hamster somatic cell hybrids and in recombinant inbred strains that the 2 genes are tightly linked on murine chromosome 2, approximately 4.7 cM distal to beta-2-microglobulin. By pulsed field gel electrophoresis, Silver et al. (1990) showed that the mouse Il1a and Il1b genes are contained in a genomic fragment of about 70 kb. Further studies suggested that Il1b lies 5-prime to Il1a, that the 2 genes are oriented in the same direction, and that they are separated by about 50 kb. From restriction mapping of the human genomic region, Nicklin et al. (1994) concluded that, relative to one terminal CpG island, the 3 genes mapped to the following intervals: IL1A was between +0 and +35 kb, IL1B between +70 and +110 kb, and IL1RN (147679) between +330 and +430 kb. Since the assignment of IL1RN to 2q14.2 appears to be the most definitive localization, the IL1A and IL1B genes can be presumably be said to be also on 2q14. Cox et al. (1998) carried out studies with multiallelic markers that grouped the 3 genes into a biallelic system for use in association studies. They identified a common, 8-locus haplotype of the IL1 gene cluster.

Hurwitz et al. (1992) studied the role of IL1 in the ovary, using a solution hybridization/RNase protection assay to test for expression of the IL1 gene, its type I receptor (IL1R; 147810), and its receptor antagonist (IL1RN). They presented findings which, taken together, revealed the existence of a complete, highly compartmentalized, hormone-dependent intraovarian IL1 system.

Since IL1 is an important cytokine in the control of the inflammatory response central to the pathology of rheumatoid arthritis (180300), Cox et al. (1999) used the combined sib-TDT (transmission/disequilibrium test) and TDT, in addition to parametric and nonparametric linkage methods, to investigate candidate genes of the IL1 gene cluster in the 2q13 region. Several tightly linked IL1 cluster markers yielded suggestive evidence for linkage in the combined TDT in those families in which affected sibs did not share 2 HLA-DRB1 alleles identical by descent. The evidence was significant in those with severe disease, as assessed by the presence of bone erosions. In contrast, there was no evidence of linkage using nonparametric linkage analysis, but parametric analysis revealed weak evidence of linkage when marker-trait disequilibrium was incorporated into the analysis. The data provided preliminary evidence for linkage of genes of the IL1 cluster to rheumatoid arthritis and suggested a possible role for this region in severe erosive disease.

Kornman et al. (1997) suggested that genetic polymorphisms of the IL1A and IL1B genes may be associated with severity of periodontitis in adult nonsmokers. The IL1B polymorphism was referred to as IL1B+3953 and the IL1A polymorphism was referred to as IL1A-889. Nonsmokers aged 40 to 60 carrying the ‘2’ allele (in either homozygous or heterozygous state) at both loci were observed to have nearly 19 times the risk of developing severe periodontitis compared to subjects homozygous for the ‘1 ’ allele at either or both of these loci. Because of the implication of interleukin-1 in adult periodontitis, Diehl et al. (1999) undertook an evaluation of the role of these IL1A and IL1B polymorphisms in early-onset periodontitis (EOP; see 170650) in 28 African-American families and 7 Caucasian-American families with 2 or more affected members. The 2 major EOP subtypes, localized juvenile periodontitis and generalized early-onset periodontitis, encompassing rapidly progressive periodontitis and generalized juvenile periodontitis, were analyzed separately and together. They obtained highly significant evidence of linkage disequilibrium for both groups of generalized EOP subjects. A similar trend was noted for the localized form. The IL1 alleles associated with high risk of EOP had been suggested previously to be correlated with low risk for severe adult periodontitis. Linkage disequilibrium with generalized EOP was equally strong for smoking and nonsmoking subjects. IL1A and IL1B polymorphisms were in strong linkage disequilibrium with each other in Caucasians but not in African Americans. Haplotype analyses evaluating both polymorphisms simultaneously indicated that the IL1B variant is likely to be more important for EOP risk. Sib pair linkage analyses, by contrast, provided only marginal support for a gene of very major effect on EOP risk attributable to these IL1 polymorphisms. Diehl et al. (1999) interpreted their results as indicating that EOP is a complex, oligogenic disorder, with interleukin-1 genetic variation contributing an important but not exclusive influence on disease risk.

NOV69 is predicted to be expressed in at least the following tissues: spleen, lymph node, thymus, tonsil and leukocyte tissues. This information was derived by determining the tissue sources of the sequences that were included in the invention including but not limited to SeqCalling sources, public EST sources, literature sources, and/or RACE sources. Further expression data for NOV69 is provided in Example 2.

The NOV69 nucleic acids and proteins of are useful in potential therapeutic applications implicated in various pathological disorders described further herein, for example, the compositions of the present invention will have efficacy for the treatment of patients suffering from: inflammatory and immune system-related diseases such as rheumatoid arthritis and inflammatory bowel disease, periodontitis, hypothyroidism, congenital, due to thyroid dysgenesis or hypoplasia; osteoarthritis of distal interphalangeal joints; selective T-cell defect; nephronophthisis, juvenile; purpura fulminans, neonatal; susceptibility to infections such as viral and bacterial; thrombophilia due to protein C deficiency; as well as other diseases, disorders and conditions.

The NOV69 nucleic acid encoding the IL1-like protein of the invention, or fragments thereof, may further be useful in diagnostic applications, wherein the presence or amount of the nucleic acid or the protein are to be assessed. The novel nucleic acid of the invention encoding a Interleukin 1 epsilon-like protein includes the nucleic acid whose sequence is provided in Table 69A or 69C, or a fragment thereof. The invention also includes a mutant or variant nucleic acid any of whose bases may be changed from the corresponding base shown in Table 69A or 69C while still encoding a protein that maintains its Interleukin 1 epsilon-like activities and physiological functions, or a fragment of such a nucleic acid. The invention further includes nucleic acids whose sequences are complementary to the sequence of Table 69A or 69C, including nucleic acid fragments that are complementary to any of the nucleic acids just described.

The invention additionally includes nucleic acids or nucleic acid fragments, or complements thereto, whose structures include chemical modifications. Such modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 2% of the bases may be so changed.

These materials are further useful in the generation of antibodies that bind immunospecifically to the substances of the invention for use in therapeutic or diagnostic methods. These antibodies may be generated according to methods known in the art, using prediction from hydrophobicity charts, as described in the “Anti-NOVX Antibodies” section below.

NOV70

NOV70 includes two OS9-like proteins, designated herein as NOV70a and NOV70b.

NOV70a

The disclosed NOV70a (alternatively referred to herein as CG56878-01) includes the 2739 nucleotide sequence (SEQ ID NO:241) shown in Table 70A. A NOV70a ORF begins with a Kozak consensus ATG initiation codon at nucleotides 86-88 and ends with a stop codon at nucleotides 2090-2092. The disclosed NOV70a maps to human chromosome 12q13.

TABLE 70A
NOV70a Nucleotide Sequence

| | |
|---|---|
| TTGCACTCTCCCACACCCTTTTCTTTTCGTCCGCTCTTCGCTTATTTCTCCCGCCGTCTC | (SEQ ID NO:241) |
| CTCTGCATAAGAAGGGGAACGAAAGATGGCGGCCGAAACGCTGCTGTCCAGTTTGTTAGG | |
| ACTGCTGCTTCTGGGACTCCTGTTACCCGCAAGTCTGACCGGCGGTGTCGGGAGCCTGAA | |
| CCTGGAGGAGCTGAGTGAGATGCGTTATGGGATCGAGATCCTCCCGTTGCCTGTCATGGG | |
| AGGGCAGAGCCAATCTTCGGACGTGGTGATTGTCTCCTCTAAGTACAAACAGCGCTATGA | |
| GTGTCGCCTGCCAGCTGGAGCTATTCACTTCCAGCGTGAAAGGGAGGAGGAAACACCTGC | |
| TTACCAAGGGCCTGGGATCCCTGAGTTGTTGAGCCCAATGAGAGATGCTCCCTGCTTGCT | |
| GAAGACAAAGGACTGGTGGACATATGAATTCTGTTATGGACGCCACATCCAGCAATACCA | |
| CATGGAAGATTCAGAGATCAAAGGTGAAGTCCTCTATCTCGGCTACTACCAATCAGCCTT | |
| CGACTGGGATGATGAAACAGCCAAGGCCTCCAAGCAGCATCGTCTTAAACGCTACCACAG | |
| CCAGACCTATGGCAATGGGTCCAAGTGCGACCTTAATGGGAGGCCCCGGGAGGCCGACGT | |
| TCGGTTCCTCTGTGACGAGGGTGCAGGTATCTCTGGGGACTACATCGATCGCGTGGACGA | |
| GCCCTTGTCCTGCTCTTATGTGCTGACCATTCGCACTCCTCGGCTCTGCCCCCACCCTCT | |
| CCTCCGGCCCCCACCCAGTGCTGCACCGCAGGCCATCCTCTGTCACCCTTCCCTACAGCC | |
| TGAGGAGTACATGGCCTACGTTCAGAGGCAAGCCGTAGACTCAAAGCAGTATGGAGATAA | |
| AATCATAGAGGAGCTGCAAGATCTAGGCCCCCAAGTGTGGAGTGAGACCAAGTCTGGGGT | |
| GGCACCCCAAAAGATGGCAGGTGCGAGCCCGACCAAGGATGACAGTAAGGACTCAGATTT | |
| CTGGAAGATGCTTAATGAGCCAGAGGACCAGGCCCCAGGAGGGGAGGAGGTGCCGGCTGA | |
| GGAGCAGGACCCAAGCCCTGAGGCAGCAGATTCAGCTTCTGGTGCTCCCAATGATTTTCA | |
| GAACAACGTGCAGGTCAAAGTCATTCGAAGCCCTGCGGATTTGATTCGATTCATAGAGGA | |
| GCTGAAAGGTGGAACAAAAAAGGGGAAGCCAAATATAGGCCAACAGCAGCCTGTGGATGA | |
| TGCTGCAGAAGTCCCTCAGAGGGAACCAGAGAAGGAAAGGGGTCATCCAGAACGGCAGAG | |
| AGAGATGGAAGAAGAGGAGGATGAGGATGAGGATGAGGATGAAGATGAGGATGAACGGCA | |
| GTTACTGGGAGAATTTGAGAAGCAACTGGAAGGGATCCTGCTTCCGTCAGACCGACACCG | |
| GCTCCGTTCGGAGGTGAAGGCTGGCATGGAGCGGGAACTGGAGAACATCATCCAGGAGAC | |
| AGAGAAAGAGCTGGACCCAGATGGGCTGAAGAAGGAGTCAGAGCGGGATCGGGCAATGCT | |
| GGCTCTCACATCCACTCTCAACAAACTCATCAAAACACTGGAGGAAAAACAGAGTCCAGA | |
| GCTGGTGAAGAAGCACAAGAAAAAGAGGGTTGTCCCCAAAAAGCCTCCCCCATCACCCCA | |
| ACCTACAGAGGAGGATCCTGAGCACAGAGTCCGGGTCCGGGTCACCAAGCTCCGTCTCGG | |
| AGGCCCTAATCAGGATCTGACTGTCCTCGAGATGAAACGGGAAAACCCACAGCTGAAACA | |
| AATCGAGGGGCTGGTGAAGGAGCTGCTGGAGAGGGAGGGACTCACAGCTGCAGGGAAAAT | |
| TGAGATCAAAATTGTCCGCCCATGGGCTGAAGGGACTGAAGAGGGTGCACGTTGGCTGAC | |
| TGATGAGGACACGAGAAACCTCAAGGAGATCTTCTTCAATATCTTGGTGCCGGGAGCTGA | |
| AGAGGCCCAGAAGGAACGCCAGCGGCAGAAAGAGCTGGAGACCAATTACCGCCGGGTGTG | |
| GGGCTCTCCAGGTGGGGAGGGCACAGGGGACCTGGACGAATTTGACTTCTGAGACCAACA | |
| CTACACTTGACCCTTCACGGAATCCAGACTCTTCCTGGACTGGCTTGCCTCCTCCCCACC | |
| TCCCCACCCTGGAACCCCTGAGGGCCAAACAGCAGAGTGGAGCTGAGCTGTGGACCTCTC | |
| GGGCAACTCTGTGGGTGTGGGGGCCCTGGGTGAATGCTGCTCCCCCTGCTGGCAGCCACC | |
| TTGAGACCTCACCGGGCCTGTGATATTTGCTCTCCTGAACTCTCACTCAATCCTCTTCCT | |
| CTCCTCTGTGGCTTTTCCTGTTATTGTCCCCTAATGATAGGATATTCCCTGCTGCCTACC | |
| TGGAGATTCAGTAGGATCTTTTGAGTGGAGGTGGGTAGAGAGAGCAAGGAGGGCAGGACA | |
| CTTAGCAGGCACTGAGCAAGCAGGCCCCCACCTGCCCTTAGTGATGTTTGGAGTCGTTTT | |
| ACCCTCTTCTATTGAATTGCCTTGGGATTTCCTTCTCCCTTTCCCTGCCCACCCTGTCCC | |
| CTACAATTTGTGCTTCTGAGTTGAGGAGCCTTCACCTCTGTTGCTGAGGAAATGGTAGAA | |
| TGCTGCCTATCACCTCCAGCACAATCCCAGCGAAAAAGGTGTGAAGCACCCACCATGTTC | |
| TTGAACAATCAGGTTTCTAAATAAACAACTGGACCATCA | |

A NOV70a polypeptide (SEQ ID NO:242) encoded by SEQ ID NO:241 is 668 amino acids in length and is presented using the one-letter amino acid code in Table 70B. The Psort profile for NOV70a predicts that this sequence has a signal peptide and is likely to be localized outside the cell with a certainty of 0.8200. In alternative embodiments, a NOV70a polypeptide is located in the nucleus with a certainty of 0.1260. The Signal P predicts a likely cleavage site for a NOV70a peptide is between positions 25 and 26, i.e., at the dash in the sequence SLT-GG.

TABLE 70B
NOV70a Polypeptide Sequence

| | |
|---|---|
| MAAETLLSSLLGLLLLGLLLPASLTGGVGSLNLEELSEMRYGIEILPLPVMGGQSQSSDV | (SEQ ID NO:242) |
| VIVSSKYKQRYECRLPAGAIHFQREREEETPAYQCPGIPELLSPMRDAPCLLKTKDWWTY | |
| EFCYGRHIQQYHMEDSEIKGEVLYLGYYQSAFDWDDETAKASKQHRLKRYHSQTYGNGSK | |
| CDLNGRPREAEVRFLCDEGAGISGDYIDRVDEPLSCSYVLTIRTPRLCPHPLLRPPPSAA | |
| PQAILCHPSLQPEEYMAYVQRQAVDSKQYGDKIIEELQDLGPQVWSETKSGVAPQKMAGA | |
| SPTKDDSKDSDFWKMLNEPEDQAPGGEEVPAEEQDPSPEAADSASGAPNDFQNNVQVKVI | |
| RSPADLIRFIEELKGGTKKGKPNIGQEQPVDDAAEVPQREPEKERGDPERQREMEEEEDE | |
| DEDEDEDEDERQLLGEFEKELEGILLPSDRDRLRSEVKAGMERELENIIQETEKELDPDG | |
| LKKESERDRAMLALTSTLNKLIKRLEEKQSPELVKKHKKKRVVPKKPPPSPQPTEEDPEH | |
| RVRVRVTKLRLGGPNQDLTVLEMKRENPQLKQIEGLVKELLEREGLTAAGKIEIKIVRPW | |
| AEGTEEGARWLTDEDTRNLKEIFFNILVPGAEEAQKERQRQKELESNYRRVWGSPGGEGT | |
| GDLDEFDF | |

NOV70b

The disclosed NOV70b (alternatively referred to herein as CG56868-04) includes the 2702 nucleotide sequence (SEQ ID NO: ) shown in Table 70C. A SEC2 ORF begins with a Kozak consensus ATG initiation codon at nucleotides 86-88 and ends with a TGA codon at nucleotides 2036-2038. The disclosed NOV70b maps to human chromosome 12q13.

TABLE 70C
NOV70b Nucleotide Sequence

| | |
|---|---|
| TTGCACTCTCCCACACCCTTTTCTTTTCGTCCGCTCTTCGCTTATTTCTCCCGCCGTCTC | (SEQ ID NO:243) |
| CTCTGCATAAGAAGGGGAACGAAAGATGGCGGCGGAAACGCTGCTGTCCAGTTTGTTAGG | |
| ACTGCTGCTTCTGGGACTCCTGTTACCCGCAAGTCTGACCGGCGGTGTCGGGAGCCTGAA | |
| CCTGGAGGAGCTGAGTGAGATGCGTTATGGGATCGAGATCCTGCCGTTGCCTGTCATGGG | |
| AGGGCAGAGCCAATCTTCGGACGTGGTGATTGTCTCCTCTAAGTACAAACAGCGCTATGA | |
| GTGTCGCCTGCCAGCTGGAGCTATTCACTTCCAGCGTGAAAGGGAGGAGGAAACACCTGC | |
| TTACCAAGGGCCTGGGATCCCTGAGTTGTTGAGCCCAATGAGAGATGCTCCCTGCTTGCT | |
| GAACACAAAGGACTGGTGGACATATGAATTCTGTTATGGACGCCACATCCAGCAATACCA | |
| CATGGAAGATTCAGAGATCAAAGGTGAAGTCCTCTATCTCCGCTACTACCAATCAGCCTT | |
| CGACTGGGATGATGAAACAGCCAAGGCCTCCAAGCAGCATCGTCTTAAACGCTACCACAG | |
| CCAGACCTATGGCAATGGGTCCAAGTCCGACCTTAATGGGAGGCCCCGGGAGGCCGAGGT | |
| TCGGTTCCTCTGTGACGAGCGTGCAGGTATCTCTGGGGACTACATCCATCGCGTGGACGA | |
| GCCCTTGTCCTGCTCTTATGTGCTGACCATTCGCACTCCTCGGCTCTGCCCCCACCCTCT | |
| CCTCCGCCCCCCACCCAGTGCTGCACCACAGGCCATCCTCTGTCACCCTTCCCTACAGCC | |
| TGAGGAGTACATGGCCTACGTTCAGAGGCAAGCCGACTCAAAGCAGTATGGAGATAAAAT | |
| CATAGAGGAGCTGCAAGATCTAGGCCCCCAAGTGTGGAGTGAGACCAAGTCTGGGGTGGC | |
| ACCCCAAAAGATGGCAGGTGCGAGCCCGACCAAGGATGACAGTAAGGACTCAGATTTCTG | |
| GAAGATGCTTAATGAGCCAGAGGACCAGGCCCCAGGAGGGGAGGAGGTGCCGGCTGAGGA | |
| GCAGCACCCAACCCCTGAGGCAGCAGATTCAGCTTCTGGTGCTCCCAATGATTTTCAGAA | |
| CAACGTGCACGTCAAAGTCATTCGAAGCCCTGCGGATTTGATTCGATTCATAGAGGAGCT | |
| GAAAGGTGGAACAAAAAACGGGAAGCCAAATATAGGCCAAGAGCAGCCTGTGGATGATGC | |
| TGCACAAGTCCCTCAGAGGGAACCAGAGAAGGAAAGGGGTGATCCAGAACGGCAGAGAGA | |
| GATGGAAGAAGAGGAGGATGAGGATGAGGATAAGATGAGGATGAACGGCAGTTACTGGGG | |
| AGAATTTGAGAAGGAACTGGAAGGGATCCTGCTTCCGTCAGACCGAGACCGGCTCCGTTC | |
| GGAGACAGAGAAAGAGCTGGACCCAGATGGGCTGAAGAAGGAGTCAGAGCGGGATCGGGC | |
| AATGCTGGCTCTCACATCCACTCTCAACAAACTCATCAAAAGACTGGAGGAAAAACAGAG | |
| TCCAGAGCTGGTGAAGAAGCACAAGAAAAAGAGGGTTGTCCCCAAAAAGCCTCCCCCATC | |
| ACCCCAACCTACAGAGGAGGATCCTGAGCACAGAGTCCGGGTCCGGGTCACCAAGCTCCG | |
| TCTCGGAGGCCCTAATCAGGATCTGACTCTCCTCGAGATGAAACGGGAAAACCCACAGCT | |
| CAAACAAATCGAGGGGCTGGTGAAGGAGCTGCTGGAGAGGGAGGGACTCACAGCTGCAGG | |
| GAAAATTGAGATCAAAATTGTCCGCCCATGGGCTGAAGGGACTGAAGAGGGTGCACGTTG | |
| GCTGACTGATGAGGACACGAGAAACCTCAAGGAGATCTTCTTCAATATCTTGGTGCCGGG | |
| AGCTGAAGAGGCCCAGAAGGAACGCCAGCGGCAGAAAGAGCTGGAGAGCAATTACCGCCG | |
| GGTGTGGGGCTCTCCAGGTGGGGAGGGCACAGGGGACCTGGACGAATTTGACTTCTGAGA | |
| CCAACACTACACTTGACCCTTCACGGAATCCAGACTCTTCCTGGACTGGCTTGCCTCCTC | |
| CCCACCTCCCCACCCTGGAACCCCTGAGGGCCAAACAGCAGAGTGGAGCTGAGCTGTGGA | |
| CCTCTCGGGCAACTCTGTGGGTGTGGGGGCCCTGGGTGAATGCTGCTGCCCCTGCTGGCA | |
| GCCACCTTGAGACCTCACCGGGCCTGTGATATTTGCTCTCCTCAACTCTCACTCAATCCT | |
| CTTCCTCTCCTCTGTGGCTTTTCCTGTTATTGTCCCCTAATGATAGGATATTCCCTGCTG | |
| CCTACCTGGAGATTCAGTAGGATCTTTTGAGTGGAGGTGGGTAGAGAGAGCAAGGAGGGC | |
| AGGACACTTAGCAGGCACTGAGCAAGCAGGCCCCCACCTGCCCTTAGTGATGTTTGGAGT | |
| CGTTTTACCCTCTTCTATGGAATTGCCTGTGGATTCCTTCTCCCTTCCCTGCCCACCGTG | |
| TCCTACAATTGTGCTCTGAGTGAGAGCCTTCCTCTCTGCTAGGAAGGTTATGTGCCTTAC | |
| TCCGCAATCGGAAAGTTAGCCACGTTCTAATCGTTATACAAGGCTAAAAAAAATAAATAT | |
| TTATACCCGTTTTTCCCTGATTTATTTTTAAATATTATATTATTTTTAATATAATTTGTG | |
| GG | |

A NOV70b polypeptide (SEQ ID NO:244) encoded by SEQ ID NO:243 is 650 amino acids in length and is presented using the one-letter amino acid code in Table 70D. The Psort profile for NOV70b predicts that this sequence has a signal peptide and is likely to be localized outside the cell with a certainty of 0.8200. In alternative embodiments, a NOV70b polypeptide is located to lysosomes with a certainty of 0.1900, or to the nucleus with a certainty of 0.1260. The Signal P predicts a likely cleavage site for a NOV70b peptide is between positions 25 and 26, i.e., at the dash in the sequence SLT-GG.

TABLE 70D
NOV70b Polypeptide Sequence

| | |
|---|---|
| MAAETLLSSLLGLLLLGLLLPASLTGGVGSLNLEELSEMRYGIEILPLPVMGGQSQSSDV | (SEQ ID NO:244) |
| VIVSSKYKQRYECRLPAGAIHFQREREEETPAYQGPGIPELLSPMRDAPCLLKTKDWWTY | |
| EFCYGRHIQQYHNEDSEIKGEVLYLGYYQSAFDWDDETAKASKQHRLKRYHSQTYGNGSK | |
| CDLNGRPREAEVRFLCDEGAGISGDYIDRVDEPLSCSYVLTIRTPRLCPHPLLRPPPSAA | |
| PQAILCHPSLQPEEYMAYVQRQADSKQYGDKIIEELQDLGPQVWSETKSGVAPQKMAGAS | |
| PTKDDSKDSDFWKMLNEPEDQAPGGEEVPAEEQDPSPEAADSASGAPNDFQNNVQVKVIR | |
| SPADLIRFIEELKGGTKKGKPNIGQEQPVDDAAEVPQREPEKERCDPERQREMEEEEDED | |
| EDKMRMNGSYWGEFEKELEGILLPSDRDRLRSETEKELDPDGLKKESERDRAMLALTSTL | |
| NKLIKRLEEKQSPELVKKHKKKRVVPKKPPPSPQPTEEDPEHRVRVRVTKLRLGGPNQDL | |
| TVLEMKRENPQLKQTEGLVKELLEREGLTAAGKIEIKIVRPWAEGTEEGARWLTDEDTRN | |
| LKEIFFNILVPGAEEAQKERQRQKELESNYRRVWGSPGGEGTGDLDEFDF | |

A BLAST analysis of NOV70 was run against the proprietary PatP GENESEQ Protein Patent database. It was found, for example, that the amino acid sequence of NOV70 had high homology to other proteins as shown in Table 70E.

TABLE 70E
BLASTX results from PatP database for NOV70

| | | Smallest |
|---|---|---|
| | | Sum |
| | High | Probability |
| Sequences producing High-scoring Segment Pairs: | Score | P(N) |
| patp: AAG76089 Human colon cancer antigen | 423 | 3.4e−52 |
| protein | | |
| patp: AAG41826 Arabidopsis thaliana protein | 298 | 2.6e−25 |
| fragment | | |
| patp: AAG41827 Arabidopsis thaliana protein | 279 | 2.9e−23 |
| fragment | | |
| patp: AAG41828 Arabidopsis thaliana protein | 279 | 2.9e−23 |
| fragment | | |
| patp: AAU32255 Novel human secreted | 232 | 3.4e−18 |
| protein #2746 | | |

In a search of sequence databases, it was found, for example, that the nucleic acid sequence of this invention has 2735 of 2739 bases (99%) identical to a gb:GENBANK-ID:HSU41635|acc:U41635.1 mRNA from Homo sapiens (Human OS-9 precurosor mRNA, complete cds). The full amino acid sequence of the protein of the invention was found to have 667 of 668 amino acid residues (99%) identical to, and 667 of 668 amino acid residues (99%) similar to, the 667 amino acid residue ptnr:SWISSPROT-ACC:Q13438 protein from Homo sapiens (Human) (PROTEIN OS-9 PRECURSOR). The sequence of this invention has 1 amino acid inserion, compared to ptnr:SWISSPROT-ACC:Q13438 protein from Homo sapiens (Human) (PROTEIN OS-9 PRECURSOR). NOV70 also has homology to the other proteins shown in the BLASTP data in Table 70F.

TABLE 70F
NOV70 BLASTP results

| Gene Index/ | Protein/ | Length | Identity | Positive | |
|---|---|---|---|---|---|
| Identifier | Organism | (aa) | (%) | (%) | Expect |
| gi\|5803109\|ref\| | amplified in | 667 | 667/668 | 667/668 | 0.0 |
| NP_006803.1\| | osteosarcoma | | (99) | (99) | |
| (NM_006812) | [Homo sapiens] | | | | |
| gi\|12653521\|gb\| | Similar to | 612 | 512/513 | 512/513 | 0.0 |
| AAH00532.1\| | amplified in | | (99) | (99) | |
| AAH00532 | osteosarcoma | | | | |
| (BC000532) | [Homo sapiens] | | | | |
| gi\|17986213\|gb\| | OS9 | 474 | 474/475 | 474/475 | 0.0 |
| AAC39523.2\| | [Homo sapiens] | | (99) | (99) | |
| (U81031) | | | | | |
| gi\|13905114\|gb\| | Unknown | 571 | 435/630 | 495/630 | 0.0 |
| AAH06844.1\| | (protein for | | (69) | (78) | |
| AAH06844 | IMAGE: | | | | |
| (BC006844) | 3598453) | | | | |
| | [Mus | | | | |
| | musculus] | | | | |
| gi\|13676348\|gb\| | Similar to | 376 | 373/374 | 374/374 | e−161 |
| AAH06506.1\| | amplified in | | (99) | (99) | |
| AAH06506 | osteosarcoma | | | | |
| (BC006506) | [Homo sapiens] | | | | |

This BLASTP data is displayed graphically in the ClustalW in Table 70G. A multiple sequence alignment is given, with the NOV70a and b proteins being shown on lines 1 and 2 in a ClustalW analysis comparing the protein of the invention with the related protein sequences shown in Table 70F.

[image]

[image]

[image]

Amplification and overexpression of genes involved in cellular growth control occur frequently in human tumors. Using a chromosome microdissection-based hybrid-selection strategy, the OS-9 gene has been identified within 12q13-15, a region frequently amplified in human cancers. The full-length OS-9 cDNA sequence consists of 2785 bp from which an open reading frame (ORF) with 667 amino-acid residues has been deduced. The predicted polypeptide is water soluble and acidic. The OS-9 gene encodes a 2.8-kb mRNA transcribed in all 16 human tissues examined, suggesting that OS-9 is ubiquitously expressed in human tissues. OS-9 is co-amplified with CDK4 in three of five sarcoma tissues. Homology analysis of the amino-acid sequence has revealed significant similarities between OS-9 and two ORFs deduced from genomic sequences in Caenorhabditis elegans and Saccharomyces cerevisiae. The region of similarity extends over 200 residues (approximately one-third of each ORF), and eight cysteines were conserved in all three ORFs. These observations suggest that this region comprises a functional domain present in a novel evolutionarily conserved gene family defined by OS-9.

The OS-9 genomic DNA has been isolated and characterized from a human BAC library. Sequencing of the genomic DNA has shown that the gene spanned approximately 30.4 kbp and had 15 exons. The 1,010 bp sequence of the 5′ upstream region has also been determined. The potential binding-sequence motifs TATA and CCAAT for general transcription factors have been found in the 5′ upstream region. Primer extension analysis has revealed two putative transcription start sites.

Three isoforms of OS-9 cDNA have been isolated from a myeloid leukemia HL-60 cDNA library and characterized. Isoform 1 consisted of 2,700 bp, from which a 667 amino acid sequence was deduced and found to be identical with that of OS-9 cDNA from osteosarcoma cells. Isoform 2 cDNA lacked a 165 nucleotide sequence in the coding region. Isoform 3 cDNA had an additional 45 bp deletion in the coding region. Isoforms 2 and 3 encode 612 and 597 amino acid polypeptides, respectively. Comparison of their cDNA sequences with the genomic structure has indicated that three isoforms are splice variants. Reverse transcription-polymerase chain reaction analysis has shown predominant expression of isoform 2 mRNA in myeloid leukemia HL-60 cells, osteosarcoma OsA-CL cells and rhabdomyosarcoma Rh30 cells. Northern blotting has revealed similar levels of expression of OS-9 gene in various tumor cell lines of sarcoma cells, carcinoma cells and myeloid leukemia cells, but 3-4 times higher expression in OsA-CL cells and Rh30 cells containing a homogeneously staining region of 12q13-15. OS-9 expression decreased in differentiation-induced HL-60 cells. The above data suggests a possible involvement of OS-9 in cell growth and tumour development.

The NOV70 disclosed in this invention is predicted to be expressed in at least the following tissues: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea, uterus, amnion, aorta, ascending colon, bone, bronchus, cartilage, cervix, colon, cornea, coronary artery, dermis, duodenum, epidermis, epididymis, hair follicles, hypothalamus, islets of langerhans, kidney cortex, liver, lung, lymph node, lymphoid tissue, esophagus, ovary, parathyroid gland, peripheral blood, pineal gland, respiratory bronchiole, retina, skin, thymus, tonsils, umbilical vein, urinary bladder, vulva, whole organism. This information was derived by determining the tissue sources of the sequences that were included in the invention including but not limited to SeqCalling sources, public EST sources, literature sources, and/or RACE sources. Further expression data for NOV70 is provided in Example 2.

The NOV70 nucleic acids and proteins of are useful in potential therapeutic applications implicated in various pathological disorders described further herein, for example, the compositions of the present invention may have efficacy for the treatment of patients suffering from leukemias, sarcomas and other types of cancer, as well as other diseases, disorders and conditions. OS-9 was co-amplified with CDK4 in three of five sarcoma tissues (Mol Carcinog April 1996;15(4):270-5). Three isoforms of OS-9 cDNA were found in a myeloid leukemia HL-60 cDNA library and reverse transcription-polymerase chain reaction analysis has shown predominant expression of isoform 2 mRNA in myeloid leukemia HL-60 cells, osteosarcoma OsA-CL cells and rhabdomyosarcoma Rh30 cells. Northern blotting has revealed similar levels of expression of OS-9 gene in various tumor cell lines of sarcoma cells, carcinoma cells and myeloid leukemia cells (J Biochem (Tokyo) May 1998;123(5):876-82).

The NOV70 nucleic acid encoding the OS-9-like protein of the invention, or fragments thereof, may further be useful in diagnostic applications, wherein the presence or amount of the nucleic acid or the protein are to be assessed. The novel nucleic acid of the invention encoding a OS-9-like protein includes the nucleic acid whose sequence is provided in 70A or 70C, or a fragment thereof. The invention also includes a mutant or variant nucleic acid any of whose bases may be changed from the corresponding base shown in 70A or 70C while still encoding a protein that maintains its OS-9-like activities and physiological functions, or a fragment of such a nucleic acid.

The invention further includes nucleic acids whose sequences are complementary to the sequence of 70A or 70C, including nucleic acid fragments that are complementary to any of the nucleic acids just described. The invention additionally includes nucleic acids or nucleic acid fragments, or complements thereto, whose structures include chemical modifications. Such modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 1% of the bases may be so changed.

The novel protein of the invention includes the OS-9-like protein whose sequence is provided in Table 70B or 70D. The invention also includes a mutant or variant protein any of whose residues may be changed from the corresponding residue shown in Table 70B or 70D while still encoding a protein that maintains its OS-9-like activities and physiological functions, or a functional fragment thereof. In the mutant or variant protein, up to about 1% of the amino acid residues may be so changed.

These materials are further useful in the generation of antibodies that bind immunospecifically to the substances of the invention for use in therapeutic or diagnostic methods. These antibodies may be generated according to methods known in the art, using prediction from hydrophobicity charts, as described in the “Anti-NOVX Antibodies” section below.

NOV71

The disclosed NOV71 (alternatively referred to herein as CG56906-01) includes the 2081 nucleotide sequence (SEQ ID NO:245) shown in Table 71A. A NOV71 ORF begins with a Kozak consensus ATG initiation codon at nucleotides 55-57 and ends with a stop codon at nucleotides 1978-1980. The disclosed NOV71 maps to human chromosome Xp11.

TABLE 71A
NOV71 Nucleotide Sequence

| | |
|---|---|
| GCGGCCGCGCCCTCGGCCTCCTCCTCTGGGGCGCCGGCCGAGGACAGCAGCGCCATGCAG | (SEQ ID NO:245) |
| GAGCTCGCTACTGAGAAGGAGGCGGACCAGAGCCACCGGCAAGACACCGTGAGCCTGCTC | |
| ACCTTCATCCTGCTGCTCACGCTCACCATCCTCACCATCTGGCTCTTCAAGCACCGCCGG | |
| GTGCGCTTTCTGCACGAGACCGGGCTGGCCATGATCTATGGGCTCATCGTTGGGGTGATC | |
| CTGAGGTATGGTACCCCTGCTACCAGTGGCCGTGACAAATCACTCAGCTGCACTCAGGAA | |
| GACAGGGCCTTCAGTACCTTATTAGTGAATGTCAGCGGAAAGTTCTTCGAATACACTCTG | |
| AAAGGAGAAATCAGTCCTGGCAAGATCAACAGCGTAGAGCAGAATGATATGCTACGGAAG | |
| GTAACATTCGATCCAGAAGTATTTTTCAACATTCTTCTGCCTCCAATTATTTTTCATGCT | |
| GGATACAGCTTAAAGAAGAGACACTTTTTCAGAAATCTTGGATCTATACTGGCCTATGCC | |
| TTCTTCGGGACTGCTGTTTCATGCTTCATTATTGGAAATCTCATGTATGGTGTGGTGAAG | |
| CTCATGAAGATTATGGGACAGCTCTCAGATAAATTTTACTACACAGATTGTCTCTTTTTT | |
| GGAGCAATCATCTCTCCCACTGACCCAGTCACTGTGCTGCCGATATTTAATGAATTGCAT | |
| GCAGACGTGGATCTTTACGCACTTCTTTTTGGAGACAGCGTCCTAAATGATGCTGTTGCC | |
| ATTGTACTGTCCTCCTCTATTGTTGCCTACCAGCCAGCCGGACTGAACACTCACGCCTTT | |
| GATGCTGCTGCCTTTTTTAAGTCAGTTGGCATTTTTCTAGGTATATTTAGTGGCTCTTTT | |
| ACCATGGGAGCTGTGACTGGTGTTGTGACTCCTCTAATATCCTTTTTACAGAATGCCAAC | |
| GTGACTAAGTTTACCAAACTGCACTGCTTCCCCCTGCTGGACACGGCGCTGTTCTTCCTC | |
| ATGTCCTGGAGCACGTTTCTCTTGGCAGAAGCCTGCGGATTTACAGGTGTTGTAGCTGTC | |
| CTTTTCTGTGGAATCACACAAGCTCATTACACCTACAACAATCTGTCGGTGGAATCAAGA | |
| AGTCGAACCAAGCAGCTCTTTGAGGTGTTACATTTCCTGCCAGAGAACTTCATCTTCTCC | |
| TACATGGGCCTGGCACTGTTTACCTTCCAGAAGCACGTTTTCAGCCCCATTTTCATCATC | |
| GGAGCTTTTGTTGCCATCTTCCTGGGCAGAGCCGCGCACATCTACCCGCTCTCCTTCTTC | |
| CTCAACTTGGGGCAGAGGCATAAGATTGGCTGGAATTTTCAACACATGATGATGTTTTCA | |
| GGCCTCAGGGGAGCAATGGCATTTGCGTTGGCCATCCGTGACACGGCATCCTATGCTCGC | |
| CAGATGATGTTCACGACCACCCTTCTCATTGTGTTCTTCACTGTCTGGATCATTGGAGGA | |
| GGCACGACACCCATGTTGTCATGGCTTAACATCAGCTTGGACGGCCCAGATTCTGCCAGA | |
| GGAAACCGGACAAAACACGAGAGCGCATGGATATTCAGCCTGTGGTACAGCTTTGATCAC | |
| AATTACCTGAAGCCCATCCTCACACACAGTGGTCCCCCACTAACCACCACGCTCCCCGCC | |
| TGGTGTGGCTTACTAGCTCGATGTCTGACCAGTCCCCAGGTGTACGATAACCAAGAGCCA | |
| CTGAGAGAGGAAGACTCTGATTTCATCCTGACCGAAGGCGACCTGACATTCACCTACGGG | |
| GACAGCACAGTGACTGCAAATGGCTCCTCAAGTTCCCACACCGCCTCCACGAGTCTGGAG | |
| GGCAGCCGGAGAACGAAGAGCAGCTCGGAGGAAGTGCTGGAGCGAGACCTGGGAATGGGA | |
| GACCAGAAGGTTTCGAGCCGGGGCACCCGCCTAGTGTTTCCCCTGGAAGATAATGCTTGA | |
| CTTTCCCCCCAACCCCTGGCGCGATGGGGTACGCTCCCGATGGGGTGAGGACAGCTGCAA | |
| GCCCTAGTGTTGTTGGAGGTGGGGCAGTGACTAGATTGAAC | |

A NOV71 polypeptide (SEQ ID NO:246) encoded by SEQ ID NO:245 is 641 amino acids in length and is presented using the one-letter amino acid code in Table 71B. The Psort profile for NOV71 predicts that this sequence is likely to be a Type III 6 membrane protein, has a signal peptide, and is likely to be localized to the plasma membrane with a certainty of 0.8200. In alternative embodiments, a NOV71 polypeptide is located to Golgibodies with a certainty of 0.4600, or to the endoplasmic reticulm (membrane) with a certainty of 0.6850. The Signal P predicts a likely cleavage site for a NOV71 peptide is between positions 37 and 38, i.e., at the dash in the sequence IWL-FK.

TABLE 71B
NOV71 Polypeptide Sequence

| | |
|---|---|
| MEELATEKEAEESHRQDSVSLLTFILLLTLTILTIWLFKHRRVRFLHETGLAMIYGLIVG | (SEQ ID NO:246) |
| VILRYGTPATSGRDKSLSCTQEDRAFSTLLVNVSGKFFEYTLKGEISPGKINSVEQNDML | |
| RKVTFDPEVFFNILLPPIIFHAGYSLKKRHFFRNLGSILAYAFLGTAVSCFIIGNLMYGV | |
| VKLMKIMGQLSDKFYYTDCLFFGAIISATDPVTVLAIFNELHADVDLYALLFGESVLNDA | |
| VAIVLSSSIVAYQPAGLNTHAFDAAAFFKSVGIFLGIFSGSFTMGAVTGVVTALISFLQN | |
| ANVTKFTKLHCFPLLETALFFLMSWSTFLLAEACGFTGVVAVLFCGITQAHYTYNNLSVE | |
| SRSRTKQLFEVLHFLAENFIESYMGLALFTFQKHVFSPIFIIGAFVAIFLGRAAHIYPLS | |
| FFLNLGRRHKIGWNFQHMMMFSGLRGAMAFALAIRDTASYARQMMFTTTLLIVFFTVWII | |
| GGGTTPMLSWLNIRLDGPDSARGNRTKQESAWIFRLWYSFDHNYLKPILTHSGPPLTTTL | |
| PAWCGLLARCLTSPQVYDNQEPLREEDSDFILTSGDLTLTYGDSTVTANGSSSSHTASTS | |
| LEGSRRTKSSSEEVLERDLGMGDQKVSSRGTRLVFPLEDNA | |

A BLAST analysis of NOV71 was run against the proprietary PatP GENESEQ Protein Patent database. It was found, for example, that the amino acid sequence of NOV71 had high homology to other proteins as shown in Table 71C.

TABLE 71C
BLASTX results from PatP database for NOV71

| | | Smallest |
|---|---|---|
| | | Sum |
| | High | Probability |
| Sequences producing High-scoring Segment Pairs: | Score | P(N) |
| patp: AAB90555 Human secreted protein, | 2410 | 5.1e−250 |
| patp: AAB90637 Human secreted protein, | 2410 | 5.1e−250 |
| patp: AAU02883 Human HsNHE-6 polypeptide - | 1693 | 5.2e−201 |
| Homo sapiens, | | |
| patp: AAB90590 Human secreted protein, | 902 | 2.1e−169 |
| patp: AAB90591 Human secreted protein, | 902 | 2.1e−169 |

In a search of sequence databases, it was found, for example, that the nucleic acid sequence of this invention has 747 of 1121 bases (66%) identical to a gb:GENBANK-ID:AF030409|acc:AF030409.1 mRNA from Homo sapiens (sodium-hydrogen exchanger 6 (NHE-6) mRNA, nuclear gene encoding mitochondrial protein). The full amino acid sequence of the protein of the invention was found to have 391 of 518 amino acid residues (75%) identical to, and 443 of 518 amino acid residues (85%) similar to, the 669 amino acid residue ptnr:SWISSNEW-ACC:Q92581 protein from Homo sapiens (Human) (SODIUM/HYDROGEN EXCHANGER 6 (NA(+)/H(+) EXCHANGER 6) (NHE-6)). NOV71 also has homology to the other proteins shown in the BLASTP data in Table 71D.

TABLE 71D
NOV71 BLASTP results

| Gene Index/ | | Length | Identity | Positive | |
|---|---|---|---|---|---|
| Identifier | Protein/Organism | (aa) | (%) | (%) | Expect |
| gi\|14211919\|ref\| | solute carrier family 9 | 725 | 631/681 | 632/681 | 0.0 |
| NP_115980.1\| | (sodium/hydrogen | | (92) | (92) | |
| (NM_032591) | exchanger), isoform 7; | | | | |
| | nonselective sodium | | | | |
| | potassium/proton | | | | |
| | exchanger [Homo sapiens] | | | | |
| gi\|3319946\|emb\| | dJ71L16.5 (KIAA0267 LIKE | 616 | 575/625 | 576/625 | 0.0 |
| CAA18155.1\| | putative Na(+)/H(+) | | (92) | (92) | |
| (AL022165) | exchanger) [Homo | | | | |
| | sapiens] | | | | |
| gi\|1665827\|dbj\| | Similar to Human Na+/H+ | 666 | 451/649 | 513/649 | 0.0 |
| BAA13449.1\| | exchanger 2 (A57644) | | (69) | (78) | |
| (D87743) | [Homo sapiens] | | | | |
| gi\|5454070\|ref\| | solute carrier family 9 | 669 | 451/649 | 513/649 | 0.0 |
| NP_006350.1\| | (sodium/hydrogen | | (69) | (78) | |
| (NM_006359) | exchanger), isoform 6 | | | | |
| | [Homo sapiens] | | | | |
| gi\|17474970\|ref\| | similar to solute | 485 | 292/412 | 305/412 | e−148 |
| XP_062645.1\| | carrier family 9 | | (70) | (73) | |
| (XM_062645) | (sodium/hydrogen | | | | |
| | exchanger), isoform 7; | | | | |
| | nonselective sodium | | | | |
| | potassium/proton | | | | |
| | exchanger (H. sapiens) | | | | |
| | [Homo sapiens] | | | | |

This BLASTP data is displayed graphically in the ClustalW in Table 71E. A multiple sequence alignment is given, with the NOV71 protein being shown on line 1 in a ClustalW analysis comparing the protein of the invention with the related protein sequences shown in Table 71D.

[image]

[image]

Table 71F lists the domain description from DOMAIN analysis results against NOV71. This indicates that the NOV71 sequence has properties similar to those of other proteins known to contain this domain.

TABLE 71F
Domain Analysis of NOV71

gnl|Pfam|pfam00999, Na_H_Exchanger, Sodium/hydrogen exchanger family. Na/H
antiporters are key transporters in maintaining the pH of actively
metobolizing cells. The molecular mechanisms of antiport are unclear. These
antiporters contain 10-12 transmembrane regions (M) at the amino-terminus and
a large cytoplasmic region at the carboxyl terminus. The transmembrane
regions M3-M12 share identity with other members of the family. The M6 and M7
regions are highly conserved. Thus, this is thought to be the region that is
involved in the transport of sodium and hydrogen ions. The cytoplasmic region
has little similarity throughout the family. SEQ ID NO:865
CD-Length = 400 residues, 88.5% aligned
Score = 276 bits (706), Expect = 3e−75

| | | | |
|---|---|---|---|
| NOV71: | 123 | VTFDPEVFFNILLPPIIFHAGYSLKKRHFFRNLGSILAYAFLGTAVSCFIIGNLMYGVVK | 182 |
| | | V D EVFF ILLPPI+F AG L R FRNLGSIL A LG + IG LMY +V | |
| Sbjct: | 46 VDLDSEVFFEILLPPILFEAGLELOLRELFRNLGSILLLALLGV7LIPAIJGIGGLMYALVP 105 | | |
| NOV71: | 183 | LMKIMGQLSDKFYYTDCLFFGAIISATDPVTVLAIFNEL-HADVDLYALLFGESVLNDAV | 241 |
| | | ++ + + L FGAI+SATDPV VLA+ EL L L+FGES+LND V | |
| Sbjct: | 106 | ILGL--------DFLAALLFGAILSATDPVAVLAVLKELGRVPKRLGTLIFGESLLNDGV | 157 |
| NOV71: | 242 | AIVLSSSIVAYQPAGLNTHAFDAAAFFKSVGIFLGIFSGSFTMGAVTGVVTALISFLQNA | 301 |
| | | A+VL + ++++ G A +A F + FL +F G +G V G + +LI | |
| Sbjct: | 158 | AVVLLAVLISFALGG----AVEAFDIFLGILSFLVVFLGGILIGLVLGYLLSLI------ | 207 |
| NOV71: | 302 | NVTKFTKLHCFPLLETALFFLMSWSTFLLAEACGFTCVVAVLFCGITQAHYTYNNLSVES | 361 |
| | | T+FT L+E L L+++ +LLAE G +G++AV G+ ++Y N+S +S | |
| Sbjct: | 208 | --TRFTFRE-DRLIEPLLVLLLAYLAYLLAEILGLSGILAVFAAGLALSNYVEANISEKS | 264 |
| NOV71: | 362 | RSRTKQLFEVLHFLAENFIFSYMCLALFTFQKHVFSPIFIIGAFVAIFLGRAAHIYPLSF | 421 |
| | | R+ K ++VL FL E IF +GL+L H ++ I+A V I L RA ++L+ | |
| Sbjct: | 265 | RTTEKYFWKVLSFLFEPLIFVLLGLSLDLSVLHNWNIALILLAIVLILLARAICVFLLTL | 324 |
| NOV71: | 422 | FLNLGRRHKICWNFQHMMMFSGLRGAMAFALAIRDTASY--ARQMMFTTTLLIVFFTVWI | 479 |
| | | LN RR KI + Q ++ + GLRGA+A ALA+ + AR ++ TT +++V TV + | |
| Sbjct: | 325 | LLNFFRREKIPFGDQLVIGWGGLRGAVALALALSGPLTSGPARDLILTTAIIVVLVTVLV | 384 |
| NOV71: | 480 | IGGGTTPMLSWLNIR 494 | |
| | | G P++ L ++ | |
| Sbjct: | 385 | QGITLKPLVKKLRVK 399 | |

Na+/H+ exchangers are integral membrane ion transporter proteins that exchange extracellular Na+ for intracellular H+ with a stoichiometry of one for one. They have multiple cellular functions, including maintenance of intracellular pH, cell volume control, and reabsorption of sodium across renal, intestinal, and other epithelia. Multiple Na+/H+ exchanger isoforms (NHE1-NHE6) exist, exhibiting considerable differences in their membrane localization, biochemical and pharmacologic properties, and responsiveness to various stimuli. For example, NHE1, the most predominant isoform expressed in heart, contributes significantly to myocardial intracellular pH. Hyperactivation of NHE1 during episodes of cardiac ischemia and reperfusion has been shown to disrupt the intracellular ion balance, leading to cardiac dysfunction and damage in several animal models, which can be prevented by pharmacological antagonists of NHEH. Increased activity of sodium/hydrogen exchange also provides a potentially important mechanism for the development of hypertension.

In 1998, Numata et al. identified the gene encoding a novel isoform of sodium/hydrogen exchanger that they called NHE6 or SLC9A6 (3). The NHE6 protein has similar topology to the other NHEs in that it has 12 putative membrane-spanning segments within the N-terminal region and a hydrophilic C terminus. However, NHE6 also has a putative mitochondrial inner membrane-targeting signal at its N terminus. The NOV71 protein is homologous to the NHE6 protein, except that it is predicted to localize to the plasma membrane instead of the mitochondrial inner membrane. The NHE6-like gene maps to human chromosome Xp11. Based on its expression pattern, NOV71 may play a role in renal or metabolic diseases and immune function through its sodium/hydrogen exchange activity at the plasma membrane.

NOV71 is predicted to be expressed in at least the following tissues: kidney, tonsils, germinal B cells, uterus, pituitary gland, brain, skeletal muscle, heart, lung, liver, pancreas, small intestine, colon, kidney, spleen, thymus, peripheral blood leukocytes, testis, ovary, placenta and prostate. This information was derived by determining the tissue sources of the sequences that were included in the invention including but not limited to SeqCalling sources, public EST sources, literature sources, and/or RACE sources. Further expression data for NOV71 is provided in Example 2.

The NOV71 nucleic acids and proteins are useful in potential therapeutic applications implicated in various pathological disorders described further herein, for example, cancer, trauma, bacterial and viral infections, in vitro and in vivo regeneration, endometriosis, fertility, hemophilia, hypercoagulation, idiopathic thrombocytopenic purpura, immunodeficiencies, graft versus host disease, diabetes, autoimmune disease, renal artery stenosis, interstitial nephritis, glomerulonephritis, polycystic kidney disease, systemic lupus erythematosus, renal tubular acidosis, IgA nephropathy, hypercalceimia, Lesch-Nyhan syndrome, endocrine dysfunctions, diabetes, obesity, growth and reproductive disorders, tonsillitis as well as other diseases, disorders and conditions.

The NOV71 nucleic acids encoding the sodium/hydrogen exchanger NHE6-like protein of the invention, or fragments thereof, may further be useful in diagnostic applications, wherein the presence or amount of the nucleic acid or the protein are to be assessed. The novel nucleic acid of the invention encoding a Sodium/Hydrogen Exchanger 6-like protein includes the nucleic acid whose sequence is provided in Table 71A, or a fragment thereof. The invention also includes a mutant or variant nucleic acid any of whose bases may be changed from the corresponding base shown in Table 71A while still encoding a protein that maintains its Sodium/Hydrogen Exchanger 6-like activities and physiological functions, or a fragment of such a nucleic acid. The invention further includes nucleic acids whose sequences are complementary to the sequence of Table 71A, including nucleic acid fragments that are complementary to any of the nucleic acids just described.

The invention additionally includes nucleic acids or nucleic acid fragments, or complements thereto, whose structures include chemical modifications. Such modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 34% of the bases may be so changed.

The novel protein of the invention includes the Sodium/Hydrogen Exchanger 6-like protein whose sequence is provided in Table 71B. The invention also includes a mutant or variant protein any of whose residues may be changed from the corresponding residue shown in Table 71B while still encoding a protein that maintains its Sodium/Hydrogen Exchanger 6-like activities and physiological functions, or a functional fragment thereof. In the mutant or variant protein, up to about 25% of the amino acid residues may be so changed.

These materials are further useful in the generation of antibodies that bind immunospecifically to the substances of the invention for use in therapeutic or diagnostic methods. These antibodies may be generated according to methods known in the art, using prediction from hydrophobicity charts, as described in the “Anti-NOVX Antibodies” section below.

NOV72

The disclosed NOV72 (alternatively referred to herein as CG56910-01) includes the 1094 nucleotide sequence (SEQ ID NO:247) shown in Table 72A. A NOV72 ORF begins Kozak consensus ATG initiation codon at nucleotides 14-16 and ends with a stop codon at nucleotides 1082-1084. The disclosed NOV72 maps to human chromosome 6.

TABLE 72A
NOV72 Nucleotide Sequence

| | |
|---|---|
| ACTTCTATAAGACATGGATAGATGCAAACATGTAGGGCGGTTAGCCCCAGGCGTCACGGG | (SEQ ID NO:247) |
| CCTGCGCAACCTGGGCAACACCTGCTACATGAACTCCATCCTCCAGGTGCTCAGCCACCT | |
| CCAGAAGTTCCGAGAATGTTTCCTCAACCTTGACCCTTCCAAAACGGAACATCTGAGTTC | |
| AAAGCACATTTCCCTCTGCCGTCAACTGCACACCCTCTTCCGAGTCATGTCGTCCGGGAA | |
| GTCGGCCCTAGTGTCGCCCTTCGCCATGCTCCACTCAGTGTGGAGCCTGATCCCTGCCTT | |
| CCGCGGCTACCACCAACAGGACGCGCAGGAATTTCTCTGCGAGCTGCTGCACAAGGTGCA | |
| GCAGGAACTCCAGTCTGAGGGCACCACACGCCGGATCCTCATCCCCTTCTCCCAGAGGAA | |
| GCTCACCAAACAGGTCTTAAAGGTGGTGAATACCATATTTCATGGGCAGCTGCTCAGTCA | |
| GGTATGTGTGGTCACATGTATATCATGCAATTACAAATCCAATACCATTGAGCCCTTTTG | |
| GGACCTATCCCTGGAATTCCCTGAACGCTATCACTGCATAGAAAAGGGGTTTGTCCCTTT | |
| GAATCAAACAGAGTGCTTGCTCACTGAGATGCTGGCCAAATTCACAGAGACAGAGGCCCT | |
| GGAAGGGAGAATCTACGCTTGTGACCAGTGTAACACTGAGTGCTGTGTGAAGCAGTTAAT | |
| GATCTACAGACTACCTCAGGTTCTCCGGCTGCACCTTAAAAGATTCCATCGAGAGAAGAT | |
| TGGGGTCCATGTCGTCTTTGACCAGGTATTAACCATGCAACCTTACTGCTGCAGGGACAT | |
| GCTCTCCTCTCTTGACAAAGAGACCTTTGCCTATGATCTCTCCGCAGTGGTCATGCATCA | |
| CGGGAAAGCGTTTGGCTCAGGACACTACACAGCCTATTGCTACAACACAGAGGGAGGTTT | |
| TTGGGTCCACTGCAATGACTCAAAGCTGAATGTATGCAGTGTCGAGGAAGTGTGCAAAAC | |
| CCAGGCCTACATCCTTTTTTACGCGCTGACTGAGATGGCGCTGAGTGAATGTGGAAGGTG | |
| CTAAGACCCAGTCT | |

A NOV72 polypeptide (SEQ ID NO:248) encoded by SEQ ID NO:247 is 356 amino acids in length and is presented using the one-letter amino acid code in Table 72B. The Psort profile for NOV72 predicts that this sequence has no signal peptide and is likely to be localized in the cytoplasm with a certainty of 0.5050. In alternative embodiments, a NOV72 polypeptide is located to lysosomes with a certainty of 0.1000, to the mitochondrial matrix space with a certainty of 0.1000, or to paroxisomal microbodies with a certainty of 0.3547.

TABLE 72B
NOV72 Polypeptide Sequence

| | |
|---|---|
| MDRCKHVGRLAPGVTGLRNLGNTCYMNSILQVLSHLQKFRECFLNLDPSKTEHLSSKHIS | (SEQ ID NO:248) |
| LCRELHTLFRVMWSGKWALVSPFANLHSVWSLIPAFRGYDQQDAQEFLCELLHKVQQELE | |
| SEGTTRRILIPFSQRKLTKQVLKVVNTIFHGQLLSQVCVVTCISCNYKSNTIEPFWDLSL | |
| EFPERYHCIEKGFVPLNQTECLLTEMLAKFTETEALEGRIYACDQCNSECCVKQLMIYRL | |
| PQVLRLHLKRFHREKIGVHVVFDQVLTMEPYCCRDMLSSLDKETFAYDLSAVVMHHGKGF | |
| GSGHYTAYCYNTEGGFWVHCNDSKLNVCSVEEVCKTQAYILFYALTEMALSECGRC | |

A BLAST analysis of NOV72 was run against the proprietary PatP GENESEQ Protein Patent database. It was found, for example, that the amino acid sequence of NOV72 had high homology to other proteins as shown in Table 72C.

TABLE 72C
BLASTX results from PatP database for NOV72

| | | Smallest |
|---|---|---|
| | | Sum |
| | High | Probability |
| Sequences producing High-scoring Segment Pairs: | Score | P(N) |
| patp: AAB92670 Human protein sequence | 1133 | 2.0e−121 |
| patp: AAB42259 Human ORFX ORF2023 | 497 | 2.7e−47 |
| polypeptide sequence | | |
| patp: AAY13115 Human secreted protein encoded | 492 | 9.1e−47 |
| by 5′ EST | | |
| patp: AAM79194 Human protein | 487 | 3.1e−46 |
| patp: AAB74672 Human protease and protease | 464 | 8.4e−44 |
| inhibitor | | |

In a search of sequence databases, it was found, for example, that the nucleic acid sequence of this invention has 215 of 342 bases (62%) identical to a gb:GENBANK-ID:AF073344|acc:AF073344.1 mRNA from Homo sapiens (ubiquitin-specific protease 3 (USP3) mRNA). The full amino acid sequence of the protein of the invention was found to have 124 of 345 amino acid residues (35%) identical to, and 183 of 345 amino acid residues (53%) similar to, the 353 amino acid residue ptnr:SWISSNEW-ACC:088623 protein from Mus musculs (Mouse) (UBIQUITIN CARBOXYL-TERMINAL HYDROLASE 2 (EC 3.1.2.15) (UBIQUITIN THIOLESTERASE 2) (UBIQUITIN-SPECIFIC PROCESSING PROTEASE 2) (DEUBIQUITINATING ENZYME 2) (41 KDA UBIQUITIN-SPECIFIC PROTEASE)). NOV72 also has homology to the other proteins shown in the BLASTP data in Table 72D.

TABLE 72D
NOV72 BLASTP results

| Gene Index/ | | | Identity | Positive | |
|---|---|---|---|---|---|
| Identifier | Protein/Organism | Length (aa) | (%) | (%) | Expect |
| gi\|15208127\|dbj\| | hypothetical protein | 550 | 282/317 | 287/317 | e−157 |
| BAB63088.1\| | [Macaca fascicularis] | | (88) | (89) | |
| (AB070143) | | | | | |
| gi\|16174134\|ref\| | similar to Unknown | 640 | 255/289 | 260/289 | e−140 |
| XP_057397.1\| | (protein for MGC: 20741) | | (88) | (89) | |
| (XM_057397) | (H. sapiens) | | | | |
| gi\|14149817\|ref\| | hypothetical protein | 712 | 221/307 | 247/307 | e−119 |
| NP_115523.1\| | DKFZp434D0127 | | (71) | (79) | |
| (NM_032147) | [Homo sapiens] | | | | |
| gi\|15451368\|dbj\| | hypothetical protein | 585 | 205/239 | 210/239 | e−108 |
| BAB64488.1\| | [Macaca fascicularis] | | (85) | (87) | |
| (AB071094) | | | | | |
| gi\|16041104\|dbj\| | hypothetical protein | 497 | 111/213 | 123/213 | 2e−45 |
| BAB69719.1\| | [Macaca fascicularis] | | (52) | (57) | |
| (AB072750) | | | | | |

This BLASTP data is displayed graphically in the ClustalW in Table 72E. A multiple sequence alignment is given, with the NOV72 protein being shown on line 1 in a ClustalW analysis comparing the protein of the invention with the related protein sequences shown in Table 72D.

[image]

[image]

[image]

Table 72F lists the domain description from DOMAIN analysis results against NOV72. This indicates that the NOV72 sequence has properties similar to those of other proteins known to contain this domain.

TABLE 72F
Domain Analysis of NOV72

gnl|Pfam|pfam00443, UCH-2, Ubiquitin carboxyl-terminal
hydrolase family 2. SEQ ID NO:866
CD-Length = 68 residues, 91.2% aligned
Score = 59.7 bits (143), Expect = 3e−10

| | | | |
|---|---|---|---|
| NOV72: | 287 | YDLSAVVMHHGKGFGSGHYTAYCYNTEGGFWVHCNDSKLNVCSVEEVCK-------TQAY | 339 |
| | | Y+L AVV+H G GHY AY G W + D K++ + EEV + + AY | |
| Sbjct: | 5 | YELYAVVVHSG-SLSGGHYIAYVKKENDG-WYKFDDDKVSRVTEEEVLEFSGGGETSSAY | 62 |
| NOV72: | 340 | ILFY 343 | |
| | | ILFY | |
| Sbjct: | 63 | ILFY 66 | |

Ubiquitin is a highly conserved 76-amino acid protein involved in the regulation of intracellular protein breakdown, cell cycle regulation, and stress response. Ubiquitin is released from degraded proteins by disassembly of the polyubiquitin chains, which is mediated by ubiquitin-specific proteases (USPs). The ubiquitin-specific proteases are a family of largely dissimilar enzymes with two major conserved sequence regions, containing either a conserved cysteine residue or two conserved histidine residues, respectively. The murine Unp oncoprotein and its human homologue, Unph, both contain regions similar to the conserved Cys and His boxes common to all the Ubps.

Unp and Unph have been shown to be active deubiquitinating enzymes, able to cleave ubiquitin from both natural and engineered linear ubiquitin-protein fusions, including the polyubiquitin precursor. Mutation of the conserved Unp Cys and His residues abolishes this activity, and identifies the likely His residue in the catalytic triad. Unp is tumorigenic when overexpressed in mice, leading to the suggestion that Unp may play a role in the regulation of ubiquitin-dependent protein degradation. It was demonstrated that the high-level expression of Unp in yeast does not disrupt the degradation of the N-end rule substrate Tyr-beta-galactosidase (betagal), the non-N-end rule substrate ubiquitin-Pro-betagal, or the degradation of abnormal, canavanine-containing proteins.

Data suggests that Unp is not a general modulator of ubiquitin-dependent proteolysis. However, Unp may have a role in the regulation of the degradation of a specific, as yet undescribed, substrate(s). The novel human Ubiquitin-Specific Protease-like Proteins of the invention contains two Ubiquitin carboxyl-terminal hydrolase domains. Therefore, it is anticipated that NOV72 has a role in regulation of specific ubiquitins and could be a potentially important target for drugs. Such drugs may have important therapeutic applications, such as treating numerous tumors.

NOV72 is predicted to be expressed in at least the following tissues: bladder and cervix. This information was derived by determining the tissue sources of the sequences that were included in the invention including but not limited to SeqCalling sources, public EST sources, literature sources, and/or RACE sources. Further expression data for NOV72 is provided in Example 2.

The NOV72 nucleic acids and proteins are useful in potential therapeutic applications implicated in various pathological disorders described further herein, for example, the compositions of the present invention will have efficacy for the treatment of patients suffering from: cystitis, incontinence, fertility, systemic lupus erythematosus, autoimmune disease, asthma, emphysema, scleroderma, allergy, ARDS, Von Hippel-Lindau (VHL) syndrome, Alzheimer's disease, stroke, tuberous sclerosis, hypercalceimia, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, neurodegeneration. as well as other diseases, disorders and conditions. The NOV72 nucleic acid encoding the ubiquitin specific protease-like protein of the invention, or fragments thereof, may further be useful in diagnostic applications, wherein the presence or amount of the nucleic acid or the protein are to be assessed.

The novel nucleic acid of the invention encoding a ubiquitin-specific protease-like protein includes the nucleic acid whose sequence is provided in Table 72A, or a fragment thereof. The invention also includes a mutant or variant nucleic acid any of whose bases may be changed from the corresponding base shown in Table 72A while still encoding a protein that maintains its ubiquitin-specific protease-like activities and physiological functions, or a fragment of such a nucleic acid.

The invention further includes nucleic acids whose sequences are complementary to the sequence of Table 72A, including nucleic acid fragments that are complementary to any of the nucleic acids just described. The invention additionally includes nucleic acids or nucleic acid fragments, or complements thereto, whose structures include chemical modifications. Such modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 38% of the bases may be so changed.

The novel protein of the invention includes the ubiquitin-specific protease-like protein whose sequence is provided in Table 72B. The invention also includes a mutant or variant protein any of whose residues may be changed from the corresponding residue shown in Table 72B while still encoding a protein that maintains its ubiquitin-specific protease-like activities and physiological functions, or a functional fragment thereof. In the mutant or variant protein, up to about 65% of the amino acid residues may be so changed.

These materials are further useful in the generation of antibodies that bind immunospecifically to the substances of the invention for use in therapeutic or diagnostic methods. These antibodies may be generated according to methods known in the art, using prediction from hydrophobicity charts, as described in the “Anti-NOVX Antibodies” section below.

NOV73

The disclosed NOV73 (alternatively referred to herein as CG56822-01) includes the 967 nucleotide sequence (SEQ ID NO:249) shown in Table 73A. A NOV73 ORF begins with a Kozak consensus ATG initiation codon at nucleotides 26-28 and ends with a stop codon at nucleotides 935-937. The disclosed NOV73 maps to human chromosome 2.

TABLE 73A
NOV73 Nucleotide Sequence

| | |
|---|---|
| GCAGGTTCTTTTACAGGGAGCCACCATGGCTGATAAATCCAAATTTATTGAATACATTGA | (SEQ ID NO:249) |
| CGAAGCTTTAGAAAAATCAAAACAAACTGCACTCTCTCATTTATTTTTCACCTATCAGGG | |
| GATTCCTTACCCCATCACCATGTGCACCTCAGAAACTTTCCAAGCGCTGGACACCTTCGA | |
| AGCCAGACATGATGACATCGTGCTAGCATCTTATCCAAAGTGCGGTTCAAACTGGATTCT | |
| CCACATTGTCAGTGAATTAATATATGCTGTTTCTAAAAAAAAGTATAAATATCCAGAATT | |
| CCCAGTTCTTGAATGTGGGGATTCAGAAAAATATCAGAGAATGAAAGGCTTTCCATCACC | |
| AAGGATTTTGGCAACTCACCTCCACTATGACAAATTACCTGGGTCTATCTTCGAGAATAA | |
| AGCCAAGATATTGGTGATATTTCGAAACCCTAAAGATACAGCAGTATCTTTTTTGCATTT | |
| CCACAACGATGTCCCCGATATTCCAAGCTATGGCTCTTGGGATGAATTCTTCAGACAGTT | |
| CATGGTGTTTTTAGTTTCTTGGGGAAGGTATTTTGATTTTGCAATCAATTGGAACAAACA | |
| TCTTGATGGCGACAATGTTAAGTTCATATTATATGAAGACCTGAAAGAGGTGAGATTATT | |
| AGGAATAAAACAGATTGCTGAGTTCTTGGGATTCTTTCTAACTGGGGAGCAAATTCAAAC | |
| TATCTCAGTCCAGAGCACCTTCCAAGCCATGCGTGCGAAGTCTCAGGACACACACCGTGC | |
| TGTCGGCCCATTCCTTTTCCGCAAAGGTAAAGTCGCAGATTGGAAAAATTTGTTCAGTGA | |
| AATTCAGAACCAGGAAATGGATGAAAAATTCAAAGAGTGCTTAGCAGGCACCTCCCTCGG | |
| AGCAAAGTTGAAGTATGAATCATATTGCCAGGGTTGATTCCAGTCAATTCAGCAGGCCTA | |
| GATTTAT | |

A NOV73 polypeptide (SEQ ID NO:250) encoded by SEQ ID NO:249 is 303 amino acids in length and is presented using the one-letter amino acid code in Table 73B. The Psort profile for NOV73 predicts that this sequence has no signal peptide and is likely to be localized to peroxisomal microbodies with a certainty of 0.7480. In alternative embodiments, a NOV73 polypeptide is located to lysosomes with a certainty of 0.1000, or to the mitochondrial matrix space with a certainty of 0.1000.

TABLE 73B
NOV73 Polypeptide Sequence

| | |
|---|---|
| MADKSKFIEYIDEALEKSKETALSHLFFTYQGIPYPITMCTSETFQALDTFEARHDDIVL | (SEQ ID NO:250) |
| ASYPKCGSNWILHIVSELIYAVSKKKYKYPEFPVLECGDSEKYQRMKGFPSPRILATHLH | |
| YDKLPGSIFENKAKILVIFRNPKDTAVSFLHFHNDVPDIPSYGSWDEFFRQFMVFLVSWG | |
| RYFDFAINWNKHLDGDNVKFILYEDLKEVRLLGIKQIAEFLGFFLTGEQIQTISVQSTFQ | |
| AMRAKSQDTHGAVGPFLFRKGKVADWKNLFSEIQNQEMDEKFKECLAGTSLGAKLKYESY | |
| CQG | |

A BLAST analysis of NOV73 was run against the proprietary PatP GENESEQ Protein Patent database. It was found, for example, that the amino acid sequence of NOV73 had high homology to other proteins as shown in Table 73C.

TABLE 73C
BLASTX results from PatP database for NOV73

| | | Smallest |
|---|---|---|
| | | Sum |
| | High | Probability |
| Sequences producing High-scoring Segment Pairs: | Score | P(N) |
| patp: AAU07758 Human novel transferase protein, | 1562 | 3.7e−160 |
| patp: AAU07760 Human novel transferase protein, | 1366 | 2.2e−139 |
| patp: AAU07765 Human novel transferase protein, | 1013 | 5.6e−102 |
| patp: AAU07763 Human novel transferase protein, | 930 | 3.5e−93 |
| patp: AAU07761 Human novel transferase protein, | 734 | 2.1e−72 |

In a search of sequence databases, it was found, for example, that the nucleic acid sequence of this invention has 427 of 643 bases (66%) identical to a gb:GENBANK-ID:AF033189|acc:AF033189.1 mRNA from Gallus gallus (Gallus gallus sulfotransferase mRNA). The full amino acid sequence of the protein of the invention was found to have 151 of 307 amino acid residues (49%) identical to, and 212 of 307 amino acid residues (69%) similar to, the 312 amino acid residue ptnr:SPTREMBL-ACC:057338 protein from Gallus gallus (Chicken) (SULFOTRANSFERASE). NOV73 also has homology to the other proteins shown in the BLASTP data in Table 73D.

TABLE 73D
NOV73 BLASTP results

| Gene Index/ | | Length | Identity | Positive | |
|---|---|---|---|---|---|
| Identifier | Protein/Organism | (aa) | (%) | (%) | Expect |
| gi\|17447308\|ref\| | similar to | 303 | 294/303 | 295/303 | e−168 |
| XP_065865.1\| | sulfotransferase (H. | | (97) | (97) | |
| (XM_065865) | sapiens) | | | | |
| | [Homo sapiens] | | | | |
| gi\|2687360\|gb\| | sulfotransferase | 312 | 151/307 | 212/307 | 4e−84 |
| AAB88818.1\| | [Gallus gallus] | | (49) | (68) | |
| (AF033189) | | | | | |
| gi\|12229955\|sp\| | SULFOTRANSFERASE K1 | 296 | 94/294 | 146/294 | 3e−35 |
| Q9WUW8\|STK1_{—} | (RSULT1C2) | | (31) | (48) | |
| RAT | | | | | |
| gi\|18079235\|ref\| | sulfotransferase family, | 296 | 92/292 | 147/292 | 5e−35 |
| NP_081211.1\| | cytosolic, 1C, member 1 | | (31) | (49) | |
| (NM_026935) | [Mus musculus] | | | | |
| gi\|11262122\|pir\|\| | hydroxyarylamine | 296 | 93/294 | 149/294 | 2e−34 |
| JC7283 | sulfotransferase (EC | | (31) | (50) | |
| | 2.8.2) 2A - rat | | | | |

This BLASTP data is displayed graphically in the ClustalW in Table 73E. A multiple sequence alignment is given, with the NOV73 protein being shown on line 1 in a ClustalW analysis comparing the protein of the invention with the related protein sequences shown in Table 73D.

[image]

[image]

Table 73F lists the domain description from DOMAIN analysis results against NOV73. This indicates that the NOV73 sequence has properties similar to those of other proteins known to contain this domain.

TABLE 73F
Domain Analysis of NOV73

gnl|Pfam|pfam00685, Sulfotransfer, Sulfotransferase protein SEQ ID NO:867
CD-Length = 269 residues, 93.3% aligned
Score = 176 bits (446), Expect = 2e−45

| | | | |
|---|---|---|---|
| NOV73: | 49 | DTFEARHDDIVLASYPKCGSNWILHIVSELIYAVSKKKYKYPE-----FPVLECGDSEK- | 102 |
| | | + F+AR DD+++A YPK G+W+ I+S L V + + P LE E | |
| Sbjct: | 18 | NCFQARPDDVLIAGYPKSGTTWLQEIL-LHPNVGDFEPSPSDPLLFRNPWLEYPKGEDW | 76 |
| NOV73: | 103 | YQRMKGFPS-PRILATHLHYDKLPGSIFENKAKILVIFRNPKDTAVSFLHFHNDVPDIPS | 161 |
| | | Y+ +K PS PR++ THL + LP S +KAKI+ + RNPKD AVS+ HF D+P+ | |
| Sbjct: | 77 | YETLKPMPSSPRLIKTHLPLELLPKSFLSSKAKIIYVLRNPKDVAVSYYHFSRSHKDLPA | 136 |
| NOV73: | 162 | Y-GSWDEFFRQFMVFLVSWGRYFDFAINWNKHLDGDNVKFILYEDLKEVRLLGIKQIAEF | 220 |
| | | G+++EF F+ V +G YFD + W + V F+ YEDLKE IK+IAEF | |
| Sbjct: | 137 | DPGTFEEFLEAFLNGKVLYGSYFDHVLGWWELRPEPQVLFLDYEDLKEDPAGEIKKIAEF | 196 |
| NOV73: | 221 | LGFFLTGEQIQTISVQSTFQAMRA---------KSQDTHGAVGPFLFRKGKVADWKNLFS | 271 |
| | | LG L+ E++ + S+F M+ +G PF RKG V DWKN F+ | |
| Sbjct: | 197 | LGLPLSEEELDKLLDHSSFFLMKLNPLSNYETLCLGKSKGRKSPF-MRKGLVGDWKNYFT | 255 |
| NOV73: | 272 | EIQNQEMDEKFKE 284 | |
| | | QN++ D+ KE | |
| Sbjct: | 256 | PEQNEKFDKVIKE 268 | |

This family includes a range of sulfotransferase proteins including flavonyl 3-sulfotransferase, aryl sulfotransferase, alcohol sulfotransferase, estrogen sulfotransferase and phenol-sulfating phenol sulfotransferase. These enzymes are responsible for the transfer of sulphate groups to specific compounds.

NOV73 is predicted to be expressed in at least the following tissues: epithelial, endothelial, muscle, and neuronal tissues. This information was derived by determining the tissue sources of the sequences that were included in the invention including but not limited to SeqCalling sources, public EST sources, literature sources, and/or RACE sources. Further expression data for NOV73 is provided in Example 2.

The novel nucleic acid of the invention encoding a sulfotransferase-like protein includes the nucleic acid whose sequence is provided in Table 73A, or a fragment thereof. The invention also includes a mutant or variant nucleic acid any of whose bases may be changed from the corresponding base shown in Table 73A while still encoding a protein that maintains its sulfotransferase-like activities and physiological functions, or a fragment of such a nucleic acid.

The invention further includes nucleic acids whose sequences are complementary to the sequence of Table 73A, including nucleic acid fragments that are complementary to any of the nucleic acids just described. The invention additionally includes nucleic acids or nucleic acid fragments, or complements thereto, whose structures include chemical modifications. Such modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 34% of the bases may be so changed.

The novel protein of the invention includes the Sulfotransferase-like protein whose sequence is provided in Table 73B. The invention also includes a mutant or variant protein any of whose residues may be changed from the corresponding residue shown in Table 73B while still encoding a protein that maintains its Sulfotransferase-like activities and physiological functions, or a functional fragment thereof. In the mutant or variant protein, up to about 51% of the amino acid residues may be so changed.

The NOV73 nucleic acids and proteins are useful in potential therapeutic applications implicated in various pathological disorders described further herein, for example, the compositions of the present invention will have efficacy for the treatment of patients suffering from: cystitis, incontinence, fertility, systemic lupus erythematosus, autoimmune disease, asthma, emphysema, scleroderma, allergy, ARDS, Von Hippel-Lindau (VHL) syndrome, Alzheimer's disease, stroke, tuberous sclerosis, hypercalceimia, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, neurodegeneration. as well as other diseases, disorders and conditions. The NOV73 nucleic acid encoding the sulfotransferase-like protein of the invention, or fragments thereof, may further be useful in diagnostic applications, wherein the presence or amount of the nucleic acid or the protein are to be assessed.

These materials are further useful in the generation of antibodies that bind immunospecifically to the substances of the invention for use in therapeutic or diagnostic methods. These antibodies may be generated according to methods known in the art, using prediction from hydrophobicity charts, as described in the “Anti-NOVX Antibodies” section below.

NOV74

The disclosed NOV74 (alternatively referred to herein as CG56775-01) includes the 732 nucleotide sequence (SEQ ID NO:251) shown in Table 74A. A NOV74 ORF begins with a Kozak consensus ATG initiation codon at nucleotides 52-54 and ends with a stop codon at nucleotides 673-675. The disclosed NOV74 maps to human chromosome 15.

TABLE 74A
NOV74 Nucleotide Sequence

(SEQ ID NO:251)

CAGACTCCCCCTTGCTGGCTCCTGCACAGAATGCCGGGCCCCACTGCTGCCATGACAGGCCCTTTCAAAC
GCTCCATGGAAGATCTCAGTGACCTGCTCTCAGACAGCAGTGGCTGCTACAGCCTCCCAAGCCAGCCCTG
CAATGAGGTCACCCTGAAGATTTACATGGGCAACACATCTGTGGATCAGGATATCCCCAAGCTTCAGAAA
CTAGGCAGTATTCATGCCCTGAATACCACTGAGGGCAGGTCTTTCATGCACATAAACAATGCCAACTTCT
CCAAGGACTCTGGCATCACCTACCTGGGCATCAAGGCCAATGAAGTGCAGGAGTTCAACCTCAGCACCTA
CTTTGAAAGGGCTACAGACTTCACTGACCACCCCTTGGCTCAAAATGGCCAGGTGCTCGTCCAGTGCTGG
GAAGGTTACAGCCATCTCCAGCTCGTTATCATGTACCTTATGATTGTCAGAAGTTGGACATCAAGTCATC
TCAGTATCATGAGGCAGAACTGTGAGATCAGCCCCAATGATGGGTTCCTGGCTCAGCTTTGCCATCTCAA
TGACAAACTAGCCAAGGAGGGGAAGGTGAAACCCTGGGGTGCCCCTACCACCTTTGCTCGAGAGGTTCAG
TGGGAGAGGCCCTGGTTGAAGGTATCCTGTGACACTGTACCCTGATCCCAGCATCACGAGCCACTTGCCC
TCAAGTCTGTCTCAACAAGTCCTGGGCCACTT

A NOV74 polypeptide (SEQ ID NO:252) encoded by SEQ ID NO:251 is 214 amino acids in length and is presented using the one-letter amino acid code in Table 74B. The Psort profile for NOV74 predicts that this sequence has no signal peptide, and is likely to be localized at the cytoplasm with a certainty of 0.4500. In alternative embodiments, a NOV74 polypeptide is located to peroxisomal microbodies with a certainty of 0.3625, or, to the mitochonrial matrix space with a certainty of 0.1000.

TABLE 74B
NOV74 Polypeptide Sequence

| | |
|---|---|
| MPGPTAATGPFKRSMEDLSDLLSDSSGCYSLPSQPCNEVTLKIYMGNTSVDQDIPKLQK | (SEQ ID NO:252) |
| LGSIHALNTTEGRSFMHINNIANFSKDSGITYLGIKANEVQEFNLSTYFERATDFTDQALA | |
| QNGQVLVQCWEGYSHLQLVIMYLMIVRSWTSSHLSIMRQNCEISPNDGFLAQLCHLNDKL | |
| AKEGKVKPWGAPTTFAREVQWERPWLKVSCDTVP | |

A BLAST analysis of NOV74 was run against the proprietary PatP GENESEQ Protein Patent database. It was found, for example, that the amino acid sequence of NOV74 had high homology to other proteins as shown in Table 74C.

TABLE 74C
BLASTX results from PatP database for NOV74

| | | Smallest |
|---|---|---|
| | | Sum |
| | High | Probability |
| Sequences producing High-scoring Segment Pairs: | Score | P(N) |
| patp: AAR56968 Human phosphatase VHR - | 664 | 5.4e−65 |
| Homo sapiens, 185 aa. | | |
| patp: AAW35330 Human cdc25B vaccinia H1 | 664 | 5.4e−65 |
| related phosphatase | | |
| patp: AAB42873 Human ORFX ORF2637 | 664 | 5.4e−65 |
| polypeptide sequence | | |
| patp: AAG67449 Amino acid sequence of a | 664 | 5.4e−65 |
| human polypeptide | | |
| patp: AAG67628 Amino acid sequence of a human | 664 | 5.4e−65 |
| protein | | |

In a search of public sequence databases, it was found, for example, that the nucleic acid sequence of this invention has 347 of 420 bases (82%) identical to a gb:GENBANK-ID:HUMDSPHS|acc:L05147.1 mRNA from Homo sapiens (Human dual specificity phosphatase tyrosine/serine mRNA). The full amino acid sequence of the protein of the invention was found to have 135 of 185 amino acid residues (72%) identical to, and 150 of 185 amino acid residues (81%) similar to, the 185 amino acid residue ptnr:SWISSNEW-ACC:P51452 protein from Homo sapiens (Human) (DUAL SPECIFICITY PROTEIN PHOSPHATASE 3 (EC 3.1.3.48) (EC 3.1.3.16) (DUAL SPECIFICITY PROTEIN PHOSPHATASE VHR)). NOV74 also has homology to the other proteins shown in the BLASTP data in Table 74D.

TABLE 74D
NOV74 BLASTP results

| Gene Index/ | | Length | Identity | Positive | |
|---|---|---|---|---|---|
| Identifier | Protein/Organism | (aa) | (%) | (%) | Expect |
| gi\|17458321\|ref\| | similar to dual | 597 | 180/207 | 180/207 | 4e−92 |
| XP_063782.1\| | specificity phosphatase | | (86) | (86) | |
| (XM_063782) | 3; vaccinia virus | | | | |
| | phosphatase VH1-related; | | | | |
| | protein tyrosine | | | | |
| | phosphatase; | | | | |
| | serine/threonine | | | | |
| | specific protein | | | | |
| | phosphatase (H. sapiens) | | | | |
| | [Homo sapiens] | | | | |
| gi\|4758208\|ref\| | dual specificity | 185 | 135/185 | 150/185 | 5e−71 |
| NP_004081.1\| | phosphatase 3; vaccinia | | (72) | (80) | |
| (NM_004090) | virus phosphatase VH1- | | | | |
| | related; protein | | | | |
| | tyrosine phosphatase; | | | | |
| | serine/threonine | | | | |
| | specific protein | | | | |
| | phosphatase | | | | |
| | [Homo sapiens] | | | | |
| gi\|1633321\|pdb\| | Chain A, Human Vh1- | 184 | 137/184 | 149/184 | 2e−70 |
| 1VHR\|A | Related Dual-Specificity | | (72) | (80) | |
| | Phosphatase | | | | |
| gi\|18158941\|pdb\| | Chain A, Human Vh1- | 184 | 133/184 | 148/184 | 3e−69 |
| 1J4X\|A | Related Dual-Specificity | | (72) | (80) | |
| | Phosphatase C124s | | | | |
| | Mutant- Peptide Complex | | | | |
| gi\|12843112\|dbj\| | DUAL SPECIFICITY PROTEIN | 185 | 126/184 | 146/184 | 3e−65 |
| BAB25864.1\| | PHOSPHATASE 3 (EC | | (68) | (78) | |
| (AK008734) | 3.1.3.48) (EC 3.1.3.16) | | | | |
| | (DUAL SPECIFICITY | | | | |
| | PROTEIN PHOSPHATASE | | | | |
| | VHR)˜putative | | | | |
| | [Mus musculus] | | | | |

This BLASTP data is displayed graphically in the ClustalW in Table 74E. A multiple sequence alignment is given, with the NOV74 protein being shown on line 1 in a ClustalW analysis comparing the protein of the invention with the related protein sequences shown in Table 74D.

[image]

[image]

[image]

Table 74F lists the domain description from DOMAIN analysis results against NOV74. This indicates that the NOV74 sequence has properties similar to those of other proteins known to contain this domain.

TABLE 74F
Domain Analysis of NOV74

gnl|Pfam|pfam00782, DSPc, Dual specificity phosphatase, catalytic domain.
Ser/Thr and Tyr protein phosphatases. The enzyme's tertiary fold is highly
similar to that of tyrosine-specific phosphatases, except for a
“recognition” region. SEQ ID NO:868
CD-Length = 139 residues. 100.0% aligned
Score = 90.5 bits (223), Expect = 9e−20

| | | | |
|---|---|---|---|
| NOV74: | 36 | PCNEVTLKIYMGNTSVDQDIPKLQKLGSIHALNTTEGRSFMHINNFSKDSGITYLGIK | 95 |
| | | +E+ +Y+G+ ++ L KLG H +N T SK+SG YL I | |
| Sbjct: | 1 | GPSEILPHLYLGSYPTASNLAFLSKLGITHVINVT--------EEVPNSKNSGFLYLHIP | 52 |
| NOV74: | 96 | ANEVQEFNLSTYFERATDFTDQALAQNGQVLVOCWEGYSHLQLVIM-YLMIVRSWTSSH- | 153 |
| | | ++ E ++S Y + A +F + A + G+VLV C G S +I+ YLM R+ + + | |
| Sbjct: | 53 | VDDNHETDISPYLDEAVEFIEDAQKGGKVLVHCQAGISRSATLIIAYLMKTRNLSLNEA | 112 |
| NOV74: | 154 | LSIMRQNCE-ISPNDGFLAQLCHLNDK 179 | |
| | | S +++ ISPN GF QL K | |
| Sbjct: | 113 | YSFVKERRPIISPNFGFKRQLIEYERK 139 | |

Mitogen-activated protein (MAP) kinase phosphatases constitute a growing family of dual specificity phosphatases thought to play a role in the dephosphorylation and inactivation of MAP kinases and are therefore likely to be important in the regulation of diverse cellular processes such as proliferation, differentiation, and apoptosis. For this reason it has been suggested that MAP kinase phosphatases may be tumor suppressors. DUSP6 (alias PYST1), one of the dual-specificity tyrosine phosphatases, is localized on 12q21, one of the regions of frequent allelic loss in pancreatic cancer. This gene is composed of three exons, and two forms of alternatively spliced transcripts are ubiquitously expressed. Although no mutations were observed in 26 pancreatic cancer cell lines, reduced expressions of the full-length transcripts were observed in some cell lines, which may suggest some role for DUSP6 in pancreatic carcinogenesis. The mitogen-induced gene, DUSP2, encodes a nuclear protein, PAC1, that acts as a dual-specific protein phosphatase with stringent substrate specificity for MAP kinase. MAP kinase phosphorylation and consequent enzymatic activation is a central and often obligatory component in signal transduction initiated by growth factor stimulation or resulting from various types of oncogenic transformation. DUSP2 downregulates intracellular signal transduction through the dephosphorylation/inactivation of MAP kinases.

NOV74 is predicted to be expressed in at least the following tissues: retina. This information was derived by determining the tissue sources of the sequences that were included in the invention including but not limited to SeqCalling sources, public EST sources, literature sources, and/or RACE sources. Further expression data for NOV74 is provided in Example 2.

The NOV74 nucleic acids and proteins of are useful in potential therapeutic applications implicated in various pathological disorders described further herein, for example, brain disorders including epilepsy, eating disorders, schizophrenia, ADD, and cancer; heart disease; blood disorders, kidney disorders, liver diseases, inflammation and autoimmune disorders including Crohn's disease, IBD, allergies, rheumatoid and osteoarthritis, inflammatory skin disorders, allergies, blood disorders; psoriasis; colon-, ovarian-, testicular-, lymphatic-, brain-, and pancreatic cancers; leukemia AIDS; thalamus disorders; metabolic disorders including diabetes and obesity; lung diseases such as asthma, emphysema, cystic fibrosis, and cancer; pancreatic disorders including pancreatic insufficiency; and prostate disorders including prostate cancer and other diseases, disorders and conditions of the like. The NOV74 nucleic acid encoding the phosphatase-like protein of the invention, or fragments thereof, may further be useful in diagnostic applications, wherein the presence or amount of the nucleic acid or the protein are to be assessed.

The novel nucleic acid of the invention encoding a dual specificity phosphatase-like protein includes the nucleic acid whose sequence is provided in Table 74A, or a fragment thereof. The invention also includes a mutant or variant nucleic acid any of whose bases may be changed from the corresponding base shown in Table 74A while still encoding a protein that maintains its dual specificity phosphatase-like activities and physiological functions, or a fragment of such a nucleic acid.

The invention further includes nucleic acids whose sequences are complementary to the sequence of Table 74A, including nucleic acid fragments that are complementary to any of the nucleic acids just described. The invention additionally includes nucleic acids or nucleic acid fragments, or complements thereto, whose structures include chemical modifications. Such modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 18% of the bases may be so changed.

The novel protein of the invention includes the dual specificity phosphatase-like protein whose sequence is provided in Table 74B. The invention also includes a mutant or variant protein any of whose residues may be changed from the corresponding residue shown in Table 74B while still encoding a protein that maintains its dual specificity phosphatase-like activities and physiological functions, or a functional fragment thereof. In the mutant or variant protein, up to about 28% of the amino acid residues may be so changed.

These materials are further useful in the generation of antibodies that bind immunospecifically to the substances of the invention for use in therapeutic or diagnostic methods. These antibodies may be generated according to methods known, in the art, using prediction from hydrophobicity charts, as described in the “Anti-NOVX Antibodies” section below.

NOV75

The disclosed NOV75 (alternatively referred to herein as CG56783-01) includes the 840 nucleotide sequence (SEQ ID NO:253) shown in Table 75A. A NOV75 ORF begins with a Kozak consensus ATG initiation codon at nucleotides 31-33 and ends with a stop codon at nucleotides 769-771. The disclosed NOV75 maps to human chromosome 1.

TABLE 75A
NOV75 Nucleotide Sequence

(SEQ ID NO:253)

AGAACCCAAGGCTCCCTGGATTTGCAGTCCATGAGCAACAAGCCCTGCTTACAGACCCCTGCGAGGAGCA
GATTCCACGAGGGGCTGGACCAAGTCTACCTTCCAAATGTGGCTGGACTCTCTGCAGCCCCCACCCAGAG
ACTGCCCATCAGGGAAGAGATGGTGCCATCAACAGGCTATGGGGAAGAGGTGGATGAGGTCTGGCCCAAT
GTCTTCATAGCTGAGAAGAGTGTGGCTGTGAACAAGGGGAGGCTGAAGAGGCTGGGAATCACCCACATTC
TGAATGCTGCGCATGGCACCGGCGTTTACACTGGCCCCGAATTCTACACTGGCCTGGAGATCCAGTACCT
GGGTGTAGAGGTGGATGACTTTCCTGAGGTGGACATTTCCCAGCATTTCCGGAAGGCGTCTGAGTTCCTG
GATGAGGCGCTGCTGACTTACAGAGGACGTTTGACCAACGTGGGATTGAATGGGTCTGTCGGTCGCCTGC
GGCGTAAGGAGTGTGTCCCACCTCGCTCGCAGGTCCTGGAGCGCACCGGCAGACCTCGAGGCGGAGCGGG
GAAAGTCCTGGTCAGCAGCGAAATGGGCATCAGCCGGTCAGCAGTGCTGGTGGTCGCCTACCTGATGATC
TTCCACAACATGGCCATCCTGGAGGCTTTGATGACCGTGCGTAAGAAGCCGGCCATCTACCCCAATGAGG
GCTTCCTGAAGCAGCTGCGGGACCTCAATGAGAAGTTGATGGAGGAGAGAGAAGAGGACTATGGCCGGTA
GGGGGGATCAGCTGAGGCTGAGGAGGGCGAGGGCACTGGGAGCATGCTCGGGGCCAGAGTGCACGCCCTG

A NOV75 polypeptide (SEQ ID NO:254) encoded by SEQ ID NO:253 is 246 amino acids in length and is presented using the one-letter amino acid code in Table 75B. The Psort profile for NOV75 predicts that this sequence has no signal peptide and is likely to be localized at the plasma membrane with a certainty of 0.7000. In alternative embodiments, a NOV75 polypeptide is located, to the endoplasmic reticulum (membrane) with a certainty of 0.1000, or to the nucleus with a certainty of 0.2000.

TABLE 75B
NOV75 Polypeptide Sequence

| | |
|---|---|
| MSNKPCLQTPGRSRFHEGLDQVYLPNVAGLSAAPTQRLPIREEMVPSRGYGEEVDEVWPN | (SEQ ID NO:254) |
| VFIAEKSVAVNKGRLKRLGITHILNAAHGTCVYTGPEFYTGLEIQYLGVEVDDFPEVDIS | |
| QHFRKASEFLDEALLTYRGRLTNVGLNGSVGRLRRKECVPPRSQVLERTGRPRCGAGKVL | |
| VSSEMGISRSAVLVVAYLMIFHNNAILEALMTVRKKRAIYPNEGFLKQLRELNEKLMEER | |
| EEDYGR | |

A BLAST analysis of NOV75 was run against the proprietary PatP GENESEQ Protein Patent database. It was found, for example, that the amino acid sequence of NOV75 had high homology to other proteins as shown in Table 75C.

TABLE 75C
BLASTX results from PatP database for NOV75

| | | Smallest |
|---|---|---|
| | | Sum |
| | High | Probability |
| Sequences producing High-scoring Segment Pairs: | Score | P(N) |
| patp: AAE04836 Human SGP018 phosphatase | 389 | 1.6e−67 |
| polypeptide | | |
| patp: AAB40919 Human ORFX ORF683 | 457 | 1.0e−55 |
| polypeptide sequence | | |
| patp: AAE04837 Human SGP003 phosphatase | 218 | 2.2e−34 |
| polypeptide | | |
| patp: AAY85620 Human dual specificity | 210 | 1.0e−32 |
| phosphatase-9 | | |
| patp: AAE04839 Human SGP060 phosphatase | 210 | 1.0e−32 |
| polypeptide | | |

In a search of sequence databases, it was found, for example, that the nucleic acid sequence of this invention has 167 of 257 bases (64%) identical to a gb:GENBANK-ID:AB027004|acc:AB027004.1 mRNA from Homo sapiens (mRNA for protein phosphatase). The full amino acid sequence of the protein of the invention was found to have 39 of 89 amino acid residues (43%) identical to, and 58 of 89 amino acid residues (65%) similar to, the 198 amino acid residue ptnr:SPTREMBL-ACC:Q9QYJ7 protein from Mus musculus (Mouse) (PROTEIN PHOSPHATASE). NOV75 also has homology to the other proteins shown in the BLASTP data in Table 75D.

TABLE 75D
NOV75 BLASTP results

| Gene Index/ | | | Identity | Positive | |
|---|---|---|---|---|---|
| Identifier | Protein/Organism | Length (aa) | (%) | (%) | Expect |
| gi\|17454087\|ref\| | similar to protein | 370 | 73/185 | 104/185 | 4e−30 |
| XP_061191.1\| | phosphatase (H. sapiens) | | (39) | (55) | |
| (XM_061191) | [Homo sapiens] | | | | |
| gi\|13128968\|ref\| | hypothetical protein | 211 | 72/187 | 105/187 | 2e−29 |
| NP_076930.1\| | MGC1136; hypothetical | | (38) | (55) | |
| (NM_024025) | protein MGC2627 | | | | |
| | [Homo sapiens] | | | | |
| gi\|15072533\|gb\| | branching-enzyme | 188 | 75/201 | 108/201 | 69e−29 |
| AAK77966.1\| | interacting dual- | | (37) | (53) | |
| (AY040091) | specificity protein | | | | |
| | phosphatase BEDP [Homo | | | | |
| | sapiens] | | | | |
| gi\|7305011\|ref\| | dual specificity | 198 | 73/187 | 106/187 | 9e−29 |
| NP_038877.1\| | phosphatase 13 | | (39) | (56) | |
| (NM_013849) | [Mus musculus] | | | | |
| gi\|17390456\|gb\| | Similar to hypothetical | 211 | 72/187 | 104/187 | 1e−28 |
| AAH18204.1\| | protein MGC1136 | | (38) | (55) | |
| AAH18204 | [Mus musculus] | | | | |
| (BC018204) | | | | | |

This BLASTP data is displayed graphically in the ClustalW in Table 75E. A multiple sequence alignment is given, with the NOV75 protein being shown on line 1 in a ClustalW analysis comparing the protein of the invention with the related protein sequences shown in Table 75D.

[image]

[image]

[image]

Table 75F lists the domain description from DOMAIN analysis results against NOV75. This indicates that the NOV75 sequence has properties similar to those of other proteins known to contain this domain.

TABLE 75F
Domain Analysis of NOV75

gnl|Pfam|pfam00782, DSPc, Dual specificity phosphatase, catalytic domain.
Ser/Thr and Tyr protein phosphatases. The enzyme's tertiary fold is highly
similar to that of tyrosine-specific phosphatases, except for a
“recognition” region. SEQ ID NO:869
CD-Length = 139 residues, 92.8% aligned
Score = 98.2 bits (243), Expect = 5e−22

| | | | |
|---|---|---|---|
| NOV75: | 56 | EVWPNVFIAEKSVAVNKGRLKRLGITHILNAAHGTGVYTGPEFYTGLEIQYLGVEVDDFP | 115 |
| | | E+ P++++ A N L +LGITH++N F YL + VDD | |
| Sbjct: | 4 | EILPHLYLGSYPTASNLAFLSKLGITHVINVTEEVPNSKNSGF------LYLHIPVDDNH | 57 |
| NOV75: | 116 | EVDISQHFRKASEFLDEALLTYRGRLTNVGLNGSVGRLRRKECVPPRSQVLERTGRPRGG | 175 |
| | | E DIS + +A EF+++A R | |
| Sbjct: | 58 | ETDISPYLDEAVEFIEDA---------------------------------------RQK | 78 |
| NOV75: | 176 | AGKVLVSSEMGISRSAVLVVAYLMIFHNMAILEALMTVRKKR-AIYPNEGFLKQ 228 | |
| | | GKVLV + GISRSA L++AYLM N+++ EA V+++R I PN GF +Q | |
| Sbjct: | 79 | GGKVLVHCQAGISRSATLITAYLMKTRNLSLNEAYSFVKERRPIISPNFGFKRQ 132 | |

Mitogen-activated protein (MAP) kinase phosphatases constitute a growing family of dual specificity phosphatases thought to play a role in the dephosphorylation and inactivation of MAP kinases and are therefore likely to be important in the regulation of diverse cellular processes such as proliferation, differentiation, and apoptosis. For this reason it has been suggested that MAP kinase phosphatases may be tumor suppressors. DUSP6 (alias PYST1), one of the dual-specificity tyrosine phosphatases, is localized on 12q21, one of the regions of frequent allelic loss in pancreatic cancer. This gene is composed of three exons, and two forms of alternatively spliced transcripts are ubiquitously expressed. Although no mutations were observed in 26 pancreatic cancer cell lines, reduced expressions of the full-length transcripts were observed in some cell lines, which may suggest some role for DUSP6 in pancreatic carcinogenesis. The mitogen-induced gene, DUSP2, encodes a nuclear protein, PAC1, that acts as a dual-specific protein phosphatase with stringent substrate specificity for MAP kinase. MAP kinase phosphorylation and consequent enzymatic activation is a central and often obligatory component in signal transduction initiated by growth factor stimulation or resulting from various types of oncogenic transformation. DUSP2 downregulates intracellular signal transduction through the dephosphorylation/inactivation of MAP kinases.

NOV75 is predicted to be expressed in at least the following tissues: heart, breast and ovarian tissue, pancreas, brain, liver, kidney, spleen, testis, ovary, and peripheral blood leukocytes. This information was derived by determining the tissue sources of the sequences that were included in the invention including but not limited to SeqCalling sources, public EST sources, literature sources, and/or RACE sources. Further expression data for NOV75 is provided in Example 2.

The NOV75 nucleic acids and proteins of are useful in potential therapeutic applications implicated in various pathological disorders described further herein, for example, brain disorders including epilepsy, eating disorders, schizophrenia, ADD, and cancer; heart disease; blood disorders, kidney disorders, liver diseases, inflammation and autoimmune disorders including Crohn's disease, IBD, allergies, rheumatoid and osteoarthritis, inflammatory skin disorders, allergies, blood disorders; psoriasis; colon-, ovarian-, testicular-, lymphatic-, brain-, and pancreatic cancers; leukemia AIDS; thalamus disorders; metabolic disorders including diabetes and obesity; lung diseases such as asthma, emphysema, cystic fibrosis, and cancer; pancreatic disorders including pancreatic insufficiency; and prostate disorders including prostate cancer and other diseases, disorders and conditions of the like. The NOV75 nucleic acid encoding the phosphatase-like protein of the invention, or fragments thereof, may further be useful in diagnostic applications, wherein the presence or amount of the nucleic acid or the protein are to be assessed.

The novel nucleic acid of the invention encoding a dual specificity phosphatase-like protein includes the nucleic acid whose sequence is provided in Table 75A, or a fragment thereof. The invention also includes a mutant or variant nucleic acid any of whose bases may be changed from the corresponding base shown in Table 75A while still encoding a protein that maintains its dual specificity phosphatase-like activities and physiological functions, or a fragment of such a nucleic acid.

The invention further includes nucleic acids whose sequences are complementary to the sequence of Table 75A, including nucleic acid fragments that are complementary to any of the nucleic acids just described. The invention additionally includes nucleic acids or nucleic acid fragments, or complements thereto, whose structures include chemical modifications. Such modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 36% of the bases may be so changed.

The novel protein of the invention includes the dual specificity phosphatase-like protein whose sequence is provided in Table 75B. The invention also includes a mutant or variant protein any of whose residues may be changed from the corresponding residue shown in Table 75B while still encoding a protein that maintains its dual specificity phosphatase-like activities and physiological functions, or a functional fragment thereof. In the mutant or variant protein, up to about 57% of the amino acid residues may be so changed.

These materials are further useful in the generation of antibodies that bind immunospecifically to the substances of the invention for use in therapeutic or diagnostic methods. These antibodies may be generated according to methods known in the art, using prediction from hydrophobicity charts, as described in the “Anti-NOVX Antibodies” section below.

NOV76

NOV76 ncludes two phospatase-like proteins, designated herein as NOV76a and NOV76b.

NOV76a

The disclosed NOV76a (alternatively referred to herein as CG56789-01) includes the 2200 nucleotide sequence (SEQ ID NO:255) shown in Table 76A. A NOV76a ORF begins with a Kozak consensus ATG initiation codon at nucleotides 61-63 and ends with a stop codon at nucleotides 2101-2103. The disclosed NOV76 maps to human chromosome 12.

TABLE 76A
NOV76a Nucleotide Sequence

(SEQ ID NO:255)

ACCATTACATCATCGTGGCAAATTAAAGAAGGAGGTGGGAAAAGAGGACTTATTGTTGTCATGGCCCATG
AGATGATTGGAACTCAAATTGTTACTGAGAGGTTGGTGGCTCTGCTGGAAAGTGGAACGGAAAAAGTGCT
GCTAATTGATAGCCGGCCATTTGTGGAATACAATACATCCCACATTTTGGAAGCCATTAATATCAACTGC
TCCAAGCTTATGAAGCGAAGGTTGCAACAGGACAAAGTGTTAATTACAGAGCTCATCCAGCATTCAGCGA
AACATAAGGTAAACCCTCAGGTTGACATTGATTGCAGTCAGAAGGTTGTAGTTTACGATCAAAGCTCCCA
AGATGTTGCCTCTCTCTCTTCAGACTGTTTTCTCACTGTACTTCTGGGTAAACTGGAGAAGAGCTTCAAC
TCTGTTCACCTGCTTGCAGGTTTATTCTTAGGTGGGTTTGCTGAGTTCTCTCGTTGTTTCCCTGCCCTCT
GTGAAGGAAAATCCACTCTAGTCCCTACCTGCATTTCTCAGCCTTGCTTACCTGTTGCCAACATTGGGCC
AACCCGAATTCTTCCCAATCTTTATCTTGGCTGCCAGCGAGATGTCCTCAACAAGGAGCTGATGCAGCAG
AATGGGATTGGTTATGTGTTAAATGCCAGCAATACCTGTCCAAAGCCTGACTTTATCCCCGAGTCTCATT
TCCTGCGTGTGCCTGTGAATGACAGCTTTTGTGAGAAAATTTTGCCGTGGTTGGACAAATCAGTAGATTT
CATTGGTAAGTTGACTTATACACAGAAAGCAAAAGCCTCCAATGGATGTGTTCTAGTGCACTGTTTAGCT
GGGATCTCCCGCTCCGCCACCATCGCTATCGCCTACATCATGAAGAGGATGGACATGTCTTTAGATGAAG
CTTACAGGAGATTTGTGAAAGAAAAAAGACCTACTATATCTCCAAACTTCAATTTTCTGGGCCAACTCCT
GGACTATGAGAAGAAGATTAAGAACCAGACTGGAGCATCAGGGCCAAACAGCAAACTCAAGCTGCTGCAC
CTGGAGAAGCCAAATGAACCTGTCCCTGCTGTCTCAGAGGCTGGACAGAAAAGCGAGACGCCCCTCAGTC
CACCCTGTGCCGACTCTGCTACCTCAGAGGCAGCAGGACAAAGGCCCGTGCATCCCGCCAGCGTGCCCAG
CGTGCCCAGCGTGCAGCCGTCGCTGTTAGAGGACAGCCCCCTGGTACAGGCGCTCAGTGGGCTGCACCTG
TCCGCAGACAGGCTGGAAGACAGCAATAAGCTCAAGCGTTCCTTCTCTCTGGATATCAAATCAGTTTCAT
ATTCAGCCAGCATGCCAGCATCCTTACATGGCTTCTCCTCATCAGAAGATGCTTTGGAATACTACAAACC
TTCCACTACTCTGGATGGGACCAACAAGCTATGCCAGTTCTCCCCTGTTCAGGCCTTATCGGAGCAGACT
CCCGAAACCAGTCCTGATAAGGAGGAAGCCAGCATCCCCAAGAAGCTGCAGACCGCCAGGCCTTCAGACA
GCCAGAGCAAGCCATTGCATTCGGTCAGAACCAGCAGCAGTGGCACCGCCCAGAGGTCCCTTTTATCTCC
ACTGCATCGAAGTGGGAGCGTCGACGACAATTACCACACCAGCTTCCTTTTCGGCCTTTCCACCAGCCAG
CAGCACCTCACCAAGTCTGCTGGCCTGGGCCTTAAGGGCTGGCACTCGGATATCTTGGCCCCCCAGACCT
CTACCCCTTCCCTGACCAGCAGCTGGTATTTTGCCACAGAGTCCTCACACTTCTACTCTGCCTCAGCCAT
CTACGCAGGCAGTGCCAGTTACTCTGCCTACAGCTGCAGCCAGCTCCCCACTTGCGGAGACCAAGTCTAT
TCTGTGCGCAGGCGCCAGAAGCCAAGTGACAGAGCTGACTCGCGGCGGAGCTGCCATGAAGAGAGCCCCT
TTGAAAAGCAGTTTAAACGCAGAAGCTCCCAAATGGAATTTGGAGAGAGCATCATGTCAGAGAACAGGTC
ACGGGAAGAGCTGGGGAAAGTGGGCAGTCAGTCTAGCTTTTCGGGCAGCATGCAAATCATTGAGGTCTCC
TGAGAAGAAAGACACTTCTGACTTCTATAGACAATTTTTTTTTCTTCTTCACAAAAAAATTCCCTGTAAA
TCTGAAATATATATATGTACATACATATAT

A NOV76a polypeptide (SEQ ID NO:256) encoded by SEQ ID NO:255 is 680 amino acids in length and is presented using the one-letter amino acid code in Table 76B. The Psort profile for NOV76a predicts that this sequence has no signal peptide and is likely to be localized at the nucleus with a certainty of 0.8800. In alternative embodiments, a NOV76a polypeptide is located to peroxisomal microbodies with a certainty of 0.3000, to the mitochondrial matrix space with a certainty of 0.1000, or to lysosomes with a certainty of 0.1000.

TABLE 76B
NOV76a Polypeptide Sequence

| | |
|---|---|
| MAHEMIGTQIVTERLVALLESGTEKVLLTDSRPFVEYNTSHILEAININCSKLMKRRLQQ | (SEQ ID NO:256) |
| DKVLITELIQHSAKHKVNAQVDIDCSQKVVVYDQSSQDVASLSSDCFLTVLLGKLEKSFN | |
| SVHLLAGLFLGGFAEFSRCFPGLCEGKSTLVPTCISQPCLPVANIGPTRILPNLYLGCQR | |
| DVLNKELMQQNGTGYVLNASNTCPKPDFIPESHFLRVPVNDSFCEKILPWLDKSVDFIGK | |
| LTYTEKAKASNGCVLVHCLAGISRSATIAIAYIMKRMDMSLDEAYRRFVKEKRPTISPNF | |
| NFLGQLLDYEKKIKNQTGASGPKSKLKLLHLEKPNEPVPAVSEGGQKSETPLSPPCADSA | |
| TSEAAGQRPVHPASVPSVPSVQPSLLEDSPLVQALSGLHLSADRLEDSNKLKRSFSLDIK | |
| SVSYSASMAASLHGFSSSEDALEYYKPSTTLDGTNKLCQFSPVQELSEQTPETSPDKEEA | |
| SIPKKLQTARPSDSQSKRLHSVRTSSSGTAQRSLLSPLHRSGSVEDNYHTSFLFCLSTSQ | |
| QHLTKSAGLGLKGWHSDILAPQTSTPSLTSSWYFATESSHFYSASAIYGGSASYSAYSCS | |
| QLPTCGDQVYSVRRRQKPSDRADSRRSWHEESPFEKQFKRRSCQMEFGESIMSENRSREE | |
| LGKVGSQSSFSGSMEIIEVS | |

NOV76b

The disclosed NOV76b (alternatively referred to herein as CG56789-02) includes the 2071 nucleotide sequence (SEQ ID NO:257) shown in Table 76C. A SEC2 ORF begins with a Kozak consensus ATG initiation codon at nucleotides 61-63 and ends with a stop codon at nucleotides 2047-2049. The disclosed NOV76b maps to human chromosome 12.

TABLE 76C
NOV76b Nucleotide Sequence

| | |
|---|---|
| ACCATTACATCATCOTGGCAAATTAAAGAAGGAGGTGGGAAAAGAGGACTTATTGTTGTC | (SEQ ID NO:257) |
| ATGGCCCATGAGATGATTGGAACTCAAATTGTTACTGAGAGGTTGGTGGCTCTGCTCGAA | |
| AGTGGAACGGAAAAAGTGCTGCTAATTGATAGCCGGCCATTTGTGGAATACAATACATCC | |
| CACATTTTGGAAGCCATTAATATCAACTGCTCCAAGCTTATGAAGCGAAGGTTGCAACAG | |
| GACAAAGTGTTAATTACAGAGCTCATCCAGCATTCAGCGAAACATAACGTTGACATTGAT | |
| TGCAGTCAGAAGGTTGTAGTTTACGATCAAAGCTCCCAAGATGTTGCCTCTCTCTCTTCA | |
| GACTGTTTTCTCACTGTACTTCTGGGTAAACTGGAGAAGAGCTTCAACTCTGTTCACCTG | |
| CTTGCAGGTGGGTTTGCTGAGTTCTCTCGTTGTTTCCCTGGCCTCTGTGAAGGAAAATCC | |
| ACTCTAGTCCCTACCTGCATTTCTCAGCCTTGCTTACCTGTTGCCAACATTGGGCCAACC | |
| CGAATTCTTCCCAATCTTTATCTTGGCTGCCACCGAGATGTCCTCAACAAGGAGCTGATG | |
| CAGCAGAATGGCATTGGTTATGTGTTAAATGCCAGCAATACCTGTCCAAAGCCTGACTTT | |
| ATCCCCGAGTCTCATTTCCTGCGTGTGCCTGTGAATGACAGCTTTTGTCAGAAAATTTTG | |
| CCGTGGTTGGACAAATCAGTAGATTTCATTGAGAAAGCAAAAGCCTCCAATGGATGTGTT | |
| CTAGTGCACTCTTTAGCTGGGATCTCCCGCTCCGCCACCATCGCTATCGCCTACATCATG | |
| AAGAGGATGGACATGTCTTTAGATGAAGCTTACAGATTTGTGAAAGAAAAAAGACCTACT | |
| ATATCTCCAACTTCAATTTTCTGGGCCAACTCCTGGACTATGAGAAGAAGAATTAAGAAC | |
| CAGACTGGAGCATCAGGGCCAAAGAGCAAACTCAAGCTGCTGCACCTGGAGAAGCCAAAT | |
| GAACCTGTCCCTGCTGTCTCAGAGCGTGGACAGAAAAGCGAGACGCCCCTCAGTCCACCC | |
| TGTGCCGACTCTGCTACCTCAGAGGCACCAGGACAAAGGCCCGTGCATCCCGCCAGCGTA | |
| CCCAGCGTGCAGCCGTCGCTGTTAGAGGACAGCCCGCTGGTACAGGCGCTCAGTGGGCTG | |
| CACCTGTCCGCAGACAGGCTGGAAGACAGCAATAAGCTCAAGCGTTCCTTCTCTCTGGAT | |
| ATCAAATCAGTTTCATATTCAGCCAGCATGGCAGCATCCTTACATGGCTTCTCCTCATCA | |
| GAAGATGCTTTGGAATACTACAAACCTTCCACTACTCTGGATGGGACCAACAAGCTATGC | |
| CAGTTCTCCCCTGTTCAGGAACTATCGGAGCAGACTCCCGAAACCAGTCCTGATAAGGAG | |
| GAAGCCAGCATCCCCAAGAAGCTGCAGACCGCCAGGCCTTCAGACAGCCAGAGCAAGCGA | |
| TTGCATTCGGTCAGAACCAGCAGCAGTGGCACCGCCCAGAGGTCCCTTTTATCTCCACTG | |
| CATCGAAGTGGGAGCGTCGAGGACAATTACCACACCAGCTTCCTTTTCGGCCTTTCCACC | |
| AGCCAGCAGCACCTCACGAAGTCTGCTGGCCTGGGCCTTAAGGGCTGGCACTCGGATATC | |
| TTGGCCCCCCAGACCTCTACCCCTTCCCTGACCAGCAGCTGGTATTTTGCCACAGAGTCC | |
| TCACACTTCTACTCTGCCTCAGCCATCTACGGAGGCAGTGCCAGTTACTCTGCCTACAGC | |
| TGCAGCCAGCTGCCCACTTGCGGAGACCAAGTCTATTCTGTGCGCAGGCGCCAGAAGCCA | |
| AGTGACAGAGCTGACTCGCGGCGGAGCTGGCATGAAGAGAGCCCCTTTGAAAAGCAGTTT | |
| AAACGCAGAAGCTGCCAAATGGAATTTGGAGAGAGCATCATGTCAGAGAACAGGTCACGG | |
| GAAGAGCTGGGGAAAGTGGGCAGTCAGTCTACCTTTTCGGGCAGCATGGAAATCATTGAG | |
| GTCTCCTGAGAAGAAAGACACTTGTGACTTC | |

A NOV76b polypeptide (SEQ ID NO:258) encoded by SEQ ID NO:257 is 662 amino acids in length and is presented using the one-letter amino acid code in Table 76D. The Psort profile for NOV76b predicts that this sequence has no signal peptide and is likely to be localized to the nucleus with a certainty of 0.8800. In alternative embodiments, a NOV76b polypeptide is located to peroxisomal microbodies with a certainty of 0.3000.

TABLE 76D
NOV76b Polypeptide Sequence

| | |
|---|---|
| MAHEMIGTQIVTERLVALLESGTEKVLLIDSRPFVEYNTSHILEAININCSKLMKRRLQQ | (SEQ ID NO:258) |
| DKVLITELIQHSAKHKVDTDCSQKVVVYDQSSQDVASLSSDCFLTVLLGKLEKSFNSVHL | |
| LAGGFAEFSRCFPGLCEGKSTLVPTCISQPCLPVANIGPTRILPNLYLGCQRDVLNKELM | |
| QQNGIGYVLNASNTCPKPDFIPESHFLRVPVNDSFCEKILPWLDKSVDFIEKAKASNGCV | |
| LVHCLAGISRSATIAIAYIMKRNDMSLDEAYRFVKEKRPTISPNFNFLGQLLDYEKKIKN | |
| QTGASGPKSKLKLLHLEKPNEPVPAVSEGGQKSETPLSPPCADSATSEAAGQRPVHPASV | |
| PSVQPSLLEDSPLVQALSGLHLSADRLEDSNKLKRSFSLDIKSVSYSASMAASLHGFSSS | |
| EDALEYYKPSTTLDGTNKLCQFSPVQELSEQTPETSPDKEEASIPKKLQTARPSDSQSKR | |
| LHSVRTSSSGTAQRSLLSPLHRSGSVEDNYHTSFLFGLSTSQQHLTKSAGLGLKGWHSDI | |
| LAPQTSTPSLTSSWYFATESSHFYSASAIYGGSASYSAYSCSQLPTCGDQVYSVRRRQKP | |
| SDRADSRRSWHEESPFEKQFKRRSCQMEFGESIMSENRSREELGKVGSQSSFSGSMEIIE | |
| VS | |

A BLAST analysis of NOV76 was run against the proprietary PatP GENESEQ Protein Patent database. It was found, for example, that the amino acid sequence of NOV76 had high homology to other proteins as shown in Table 76E.

TABLE 76E
BLASTX results from PatP database for NOV76

| | | Smallest |
|---|---|---|
| | | Sum |
| | High | Probability |
| Sequences producing High-scoring Segment Pairs: | Score | P(N) |
| patp: AAE04834 Human SGP002 phosphatase | 3360 | 0.0 |
| polypeptide | | |
| patp: AAU09016 Human dual specificity | 3360 | 0.0 |
| phosphatase 21117 | | |
| patp: AAB20325 Human protein phosphatase | 2963 | 1.3e−308 |
| and kinase protein | | |
| patp: AAM25744 Human protein sequence | 2860 | 1.1e−297 |
| patp: AAW29150 Dual-specific murine thr-tyr | 1088 | 1.5e−125 |
| phosphatase | | |

In a search of sequence databases, it was found, for example, that the nucleic acid sequence of this invention has 1149 of 1150 bases (99%) identical to a gb:GENBANK-ID:AB052156|acc:AB052156.1 mRNA from Homo sapiens (MKP-7 mRNA for MAPK phosphatase-7). The full amino acid sequence of the protein of the invention was found to have 662 of 665 amino acid residues (99%) identical to, and 662 of 665 amino acid residues (99%) similar to, the 665 amino acid residue ptnr:SPTREMBL-ACC:Q9BY84 protein from Homo sapiens (Human) (MAPK PHOSPHATASE-7). NOV76 also has homology to the other proteins shown in the BLASTP data in Table 76F.

TABLE 76F
NOV76 BLASTP results

| Gene Index/ | | Length | Identity | Positive | |
|---|---|---|---|---|---|
| Identifier | Protein/Organism | (aa) | (%) | (%) | Expect |
| gi\|12697945\|dbj\| | KIAA1700 protein | 690 | 665/680 | 665/680 | 0.0 |
| BAB21791.1\| | [Homo sapiens] | | (97) | (97) | |
| (AB051487) | | | | | |
| gi\|14756395\|ref\| | MAPK phosphatase-7 [Homo | 665 | 665/680 | 665/680 | 0.0 |
| XP_039106.1\| | sapiens] | | (97) | (97) | |
| (XM_039106) | | | | | |
| gi\|16550836\|dbj\| | unnamed protein product | 665 | 664/680 | 664/680 | 0.0 |
| BAB71060.1\| | [Homo sapiens] | | (97) | (97) | |
| (AK055973) | | | | | |
| gi\|13990989\|dbj\| | MAP kinase phosphatase-7 | 660 | 601/680 | 628/680 | 0.0 |
| BAB47240.1\| | [Mus musculus] | | (88) | (91) | |
| (AB052157) | | | | | |
| gi\|13625393\|gb\| | map kinase phosphatase-M | 677 | 533/644 | 568/644 | 0.0 |
| AAK35052.1\| | A1 isoform | | (82) | (87) | |
| AF345951_1 | [Mus musculus] | | | | |
| (AF345951) | | | | | |

This BLASTP data is displayed graphically in the ClustalW in Table 76G. A multiple sequence alignment is given, with the NOV76 protein being shown on line 1 in a ClustalW analysis comparing the protein of the invention with the related protein sequences shown in Table 76F.

[image]

[image]

[image]

Table 76H lists the domain description from DOMAIN analysis results against NOV76. This indicates that the NOV76 sequence has properties similar to those of other proteins known to contain this domain.

TABLE 76H
Domain Analysis of NOV76

gnl|Pfam|pfam00782, DSPc, Dual specificity phosphatase, catalytic domain.
Ser/Thr and Tyr protein phosphatases. The enzyme's tertiary fold is highly
similar to that of tyrosine-specific phosphatases, except for a “recognition”
region. SEQ ID NO:870
CD-Length = 139 residues, 100.0% aligned
Score = 172 bits (436), Expect = 6e−44

| | | | |
|---|---|---|---|
| NOV76: | 166 | GPTRILPNLYLGCQRDVLNKELMQQNGIGYVLNASNTCPKPDFIPESHFLRVPVNDSFCE | 225 |
| | | GP+ ILP+LYLG N + + GI +V+N + P +L +PV+D+ | |
| Sbjct: | 1 | GPSEILPHLYLGSYPTASNLAFLSKLGITHVINVTEEVPNSKNSGFLYLHIPVDDNHET | 59 |
| NOV76: | 226 | KILPWLDKSVDFIGKLTYTEKAKASNGCVLVHCLAGISRSATIAIAYIMKRMDMSLDEAY | 285 |
| | | I P+LD++V+FI E A+ G VLVHC AGISRSAT+ IAY+MK ++SL+EAY | |
| Sbjct: | 60 | DISPYLDEAVEFI------EDARQKGGKVLVHCQAGISRSATLIIAYLMKTRNLSLNEAY | 113 |
| NOV76: | 286 | RRFVKEKRPTISPNFNFLGQLLDYEKK 312 | |
| | | FVKE+RP ISPNF F QL++YE+K | |
| Sbjct: | 114 | -SFVKERRPIISPNFGFKRQLIEYERK 139 | |

Mitogen-activated protein kinases (MAPKs) are inactivated via dephosphorylation of either the threonine or tyrosine residue or both in the P-loop catalyzed by protein phosphatases which include serine/threonine phosphatases, tyrosine phosphatases, and dual specificity phosphatases. Nine members of the dual specificity phosphatases specific for MAPKs, termed MKPs, have been reported. Each member has its own substrate specificity, tissue distribution, and subcellular localization. MKP-7 is most similar to hVH5, a member of previously known MKPs, in the primary structure. MKP-7 is predominantly localized in the cytoplasm when expressed in cultured cells, whereas hVH5 is both in the nucleus and the cytoplasm. MKP-7 binds to and inactivates p38 MAPK and JNK/SAPK, but not ERK. Furthermore, MKPs have the substrate specificity toward the isoforms of the p38 family (alpha, beta, gamma, and delta). MKP-7 binds to and inactivates p38 alpha and -beta, but not gamma or delta. MKP-5 and CL100/MKP-1 also bind to p38 alpha and -beta, but not gamma or delta.

NOV76 is predicted to be expressed in at least the following tissues: blood, brain, CNS, colon, heart, kidney, lung, and stomach. This information was derived by determining the tissue sources of the sequences that were included in the invention including but not limited to SeqCalling sources, public EST sources, literature sources, and/or RACE sources. Further expression data for NOV76 is provided in Example 2.

The NOV76 nucleic acids and proteins of are useful in potential therapeutic applications implicated in various pathological disorders described further herein, for example, cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, transplantation, hypercalceimia, ulcers, Hirschsprung's disease, Crohn's Disease, anemia, ataxia-telangiectasia, autoimmune disease, immunodeficiencies, Von Hippel-Lindau (VHL) syndrome, Alzheimer's disease, stroke, Parkinson's disease, Huntington's disease, cerebral palsy, epilepsy, Lesch-Nyhan syndrome, multiple sclerosis, leukodystrophies, behavioral disorders, addiction, anxiety, pain, neurodegeneration, neuroprotection, systemic lupus erythematosus, asthma, emphysema, allergy, ARDS, diabetes, renal artery stenosis, interstitial nephritis, glomerulonephritis, polycystic kidney disease, renal tubular acidosis, IgA nephropathy, as well as other diseases, disorders and conditions. NOV76 nucleic acids encoding the MAP kinase-like protein of the invention, or fragments thereof, may further be useful in diagnostic applications, wherein the presence or amount of the nucleic acid or the protein are to be assessed.

The novel nucleic acid of the invention encoding a phosphatase-like protein includes the nucleic acid whose sequence is provided in Table 76A or 76C, or a fragment thereof. The invention also includes a mutant or variant nucleic acid any of whose bases may be changed from the corresponding base shown in Table 76A or 76C while still encoding a protein that maintains its phosphatase-like activities and physiological functions, or a fragment of such a nucleic acid. The invention further includes nucleic acids whose sequences are complementary to the sequence of Table 76A or 76C, including nucleic acid fragments that are complementary to any of the nucleic acids just described.

The invention additionally includes nucleic acids or nucleic acid fragments, or complements thereto, whose structures include chemical modifications. Such modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 1% of the bases may be so changed.

The novel protein of the invention includes the phosphatase-like protein whose sequence is provided in Table 76B or 76D. The invention also includes a mutant or variant protein any of whose residues may be changed from the corresponding residue shown in Table 76B or 76D while still encoding a protein that maintains its phosphatase-like activities and physiological functions, or a functional fragment thereof. In the mutant or variant protein, up to about 1% of the amino acid residues may be so changed.

These materials are further useful in the generation of antibodies that bind immunospecifically to the substances of the invention for use in therapeutic or diagnostic methods. These antibodies may be generated according to methods known in the art, using prediction from hydrophobicity charts, as described in the “Anti-NOVX Antibodies” section below.

NOV77

The disclosed NOV77 (alternatively referred to herein as CG56804-01) includes the 881 nucleotide sequence (SEQ ID NO:259) shown in Table 77A. A NOV77 ORF begins with a Kozak consensus ATG initiation codon at nucleotides 61-63 and ends with a stop codon at nucleotides 769-771. The disclosed NOV77 maps to human chromosome 14.

TABLE 77A
NOV77 Nucleotide Sequence

(SEQ ID NO:259)

GAGGGTCGGCCGGCTGTGTAACACTCTCCCACCCCACCCACCAGCCCGCCGGCCAGCACCATGGAGGACG
TGAACCTGGAGTTCCCTTCCCTTCCACAGTGCAAGGAAGACGCCGAGGAGTGGACCTACCCTGAGTGGAC
CTACCCTATGAGACGAGAGATGCAGGAAATTTTACCTGGATTGTTCTTAGGCCCATATTCATCTGCTATG
AAAAGCAAGGTACTACCTGTACTACAGAAACATGGAATAACCCATATAATATGCATACGACAAAATATTG
AAGCAAACTTTATTAAACCAAACTTTCAGCAGTTATTTAGGTATTTAGTCCTGGATATTGCAGATAATCC
AGTTGAAAATATAATACGTTTTTTCCCTATGTTTTGCCTCCAGACTAAGGAATTTATTGATCGGAGCTTA
CAAATGGGAGGTAAAGTTCTTGTGCATGGAAATGCAGGGATCTCCAGAAGTGCAGCCTTTGTTATTGCAT
ACATTATGGAAACATTTGGAATGAAGTACAGGTTCAGAGATGCTTTTGCTTATGTTCAAGAAAGAAGATT
TTGTATTAATCCTAATGCTGGATTTGTCCATCAACTTCAGGATATGAAGCCATCTACCTAGCAAAAATTA
ACAATACAGATGATGTCACCACTCCAGATAGAAAGGTCATTATCTGTTCATTCTGGTACCACAGGTGGCA
GTTTGAAGAGAACACATGAAGAAGAGGATGATTTTGGAACCATGCAAGTGGCGACTGCACAGAATGGCTG
ACTTGAAGAGCAACATCATAGAGTGTGAATTTCTATTTGGGAAGGAGAAAATACAAGAGAAAATTATAAT
GTAAAATGGTAAAAACATAAGTAGTTTTTTTTTCAATTACA

A NOV77 polypeptide (SEQ ID NO:260) encoded by SEQ ID NO:259 is 236 amino acids in length and is presented using the one-letter amino acid code in Table 77B. The Psort profile for NOV77 predicts that this sequence has no signal peptide and is likely to be localized at the cytoplasm with a certainty of 0.6036. In alternative embodiments, a NOV77 polypeptide is located to lysosomes with a certainty of 0.2040.

TABLE 77B
NOV77 Polypeptide Sequence

| | |
|---|---|
| MEDVKLEFPSLPQCKEDAEEWTYPEWTYPMRREMQEILPGLFLGPYSSAMKSKVLPVLQK | (SEQ ID NO:260) |
| HGITHIICIRQNIEANFIKPNFQQLFRYLVLDIADNPVENIIRFFPMFCLQTKEFIDGSL | |
| QMGGKVLVHGNAGISRSAAFVIAYIMETFGMKYRFRDAFAYVQERRFCINPNAGFVHQLQ | |
| EYEAIYLAKLTIQMMSPLQIERSLSVHSGTTGGSLKRTHEEEDDFGTMQVATAQNG | |

A BLAST analysis of NOV77 was run against the proprietary PatP GENESEQ Protein Patent database. It was found, for example, that the amino acid sequence of NOV77 had high homology to other proteins as shown in Table 77C.

TABLE 77C
BLASTX results from PatP database for NOV77

| | | Smallest |
|---|---|---|
| | | Sum |
| | High | Probability |
| Sequences producing High-scoring Segment Pairs: | Score | P(N) |
| patp: AAM39734 Human polypeptide | 1099 | 4.3e−111 |
| patp: AAM41520 Human polypeptide | 1099 | 4.3e−111 |
| patp: AAE08552 Human phosphatase protein - | 1099 | 4.3e−111 |
| Homo sapiens | | |
| patp: AAU09017 Human dual specificity | 1099 | 4.3e−111 |
| phosphatase 38692 | | |
| patp: AAY68795 Amino acid sequence of a human | 210 | 6.9e−17 |
| protein | | |

In a search of sequence databases, it was found, for example, that the nucleic acid sequence of this invention has 228 of 249 bases (91%) identical to a gb:GENBANK-ID:MMU349731|acc:U34973.1 mRNA from Mus musculus (protein tyrosine phosphatase-like mRNA, unspliced c-terminal product and spliced c-terminal end STYX). The full amino acid sequence of the protein of the invention was found to have 214 of 236 amino acid residues (90%) identical to, and 221 of 236 amino acid residues (93%) similar to, the 223 amino acid residue ptnr:SPTREMBL-ACC:Q60970 protein from Mus musculus (Mouse) (PROTEIN TYROSINE PHOSPHATASE-LIKE). NOV77 also has homology to the other proteins shown in the BLASTP data in Table 77D.

TABLE 77D
NOV77 BLASTP results

| Gene Index/ | | | Identity | Positive | |
|---|---|---|---|---|---|
| Identifier | Protein/Organism | Length (aa) | (%) | (%) | Expect |
| gi\|17476793\|ref\| | similar to putative (H. | 223 | 223/236 | 223/236 | e−118 |
| XP_058659.1\| | sapiens) [Homo sapiens] | | (94) | (94) | |
| (XM_058659) | | | | | |
| gi\|12833088\|dbj\| | phosphoserine/threonine/ | 223 | 215/236 | 221/236 | e−116 |
| BAB22384.1\| | tyrosine interaction | | (91) | (93) | |
| (AK002822) | protein˜putative | | | | |
| | [Mus musculus] | | | | |
| gi\|2137698\|pir\|\| | protein tyrosine | 233 | 214/236 | 221/236 | e−116 |
| I49365 | phosphatase - mouse | | (90) | (92) | |
| gi\|9789981\|ref\| | phosphoserine/threonine/ | 205 | 163/180 | 167/180 | 3e−87 |
| NP_062611.1\| | tyrosine interaction | | (90) | (92) | |
| (NM_019637) | protein; STNS | | | | |
| | (alternatively spliced | | | | |
| | intron of Styx); protein | | | | |
| | tyrosine phosphatase- | | | | |
| | like unspliced c- | | | | |
| | terminal product and | | | | |
| | spliced c-terminal end | | | | |
| | STYX [Mus musculus] | | | | |
| gi\|1842088\|gb\| | tyrosine phosphatase- | 66 | 66/68 | 66/68 | 8e−30 |
| AAB47561.1\| | like protein homolog | | (97) | (97) | |
| (U87169) | hSTYXb [Homo sapiens] | | | | |

This BLASTP data is displayed graphically in the ClustalW in Table 77E. A multiple sequence alignment is given, with the NOV77 protein being shown on line 1 in a ClustalW analysis comparing the protein of the invention with the related protein sequences shown in Table 77D.

[image]

[image]

Table 77F lists the domain description from DOMAIN analysis results against NOV77. This indicates that the NOV77 sequence has properties similar to those of other proteins known to contain this domain.

TABLE 77F
Domain Analysis of NOV77

gnl|Smart|smart00195, DSPc, Dual specificity phosphatase, catalytic domain
SEQ ID NO:871
CD-Length = 139 residues, 98.6% aligned
Score = 142 bits (358), Expect = 2e−35

| | | | |
|---|---|---|---|
| NOV77: | 33 | EMQEILPGLFLGPYSSANKSKVLPVLQICHGITHIICIRQNIEANFIKPNFQQLFRYLVLD | 92 |
| | | EILP L+LG YS A L +L+K GITH+I + + + + F YL + | |
| Sbjct: | 1 | GPSEILPHLYLGSYSDASN---LALLKKLGITHVINV-----TEEVPNSNKSGFLYLGIP | 52 |
| NOV77: | 93 | IADNPVENIIRFFPMFCLQTKEFIDGSLQMGGKVLVHGNAGISRSAAFVIAYIMETFGMK | 152 |
| | | + DN I + P EFI+ + +GGKVLVH AG+SRSA +IAY+M+ M | |
| Sbjct: | 53 | VDDNTETKISPYLPE----AVEFIEDAEKKGGKVLVHCQAGVSRSATLIIAYLMKYRNMS | 108 |
| NOV77: | 153 | YRFRDAFAYVQERRFCINPNAGFVHQLQEYE 183 | |
| | | DA+ +V+ERR I+PN GF QL EYE | |
| Sbjct: | 109 | L--NDAYDFVKERRPIISPNFGFLRQLIEYE 137 | |

Mitogen-activated protein (MAP) kinase phosphatases constitute a growing family of dual specificity phosphatases thought to play a role in the dephosphorylation and inactivation of MAP kinases and are therefore likely to be important in the regulation of diverse cellular processes such as proliferation, differentiation, and apoptosis. For this reason it has been suggested that MAP kinase phosphatases may be tumor suppressors.

NOV77 is predicted to be expressed in at least the following tissues: lung, lymphoid tissue, spleen, tonsils, whole organism. This information was derived by determining the tissue sources of the sequences that were included in the invention including but not limited to SeqCalling sources, public EST sources, literature sources, and/or RACE sources. Further expression data for NOV77 is provided in Example 2.

The NOV77 nucleic acids and proteins of are useful in potential therapeutic applications implicated in various pathological disorders described further herein, for example, brain disorders including epilepsy, eating disorders, schizophrenia, ADD, and cancer; heart disease; blood disorders, kidney disorders, liver diseases, inflammation and autoimmune disorders including Crohn's disease, IBD, allergies, rheumatoid and osteoarthritis, inflammatory skin disorders, allergies, blood disorders; psoriasis; colon-, ovarian-, testicular-, lymphatic-, brain-, and pancreatic cancers; leukemia AIDS; thalamus disorders; metabolic disorders including diabetes and obesity; lung diseases such as asthma, emphysema, cystic fibrosis, and cancer; pancreatic disorders including pancreatic insufficiency; and prostate disorders including prostate cancer and other diseases, disorders and conditions of the like. The NOV77 nucleic acid encoding the MAP kinase-like protein of the invention, or fragments thereof, may further be useful in diagnostic applications, wherein the presence or amount of the nucleic acid or the protein are to be assessed.

The novel nucleic acid of the invention encoding a dual specificity phosphatase-like protein includes the nucleic acid whose sequence is provided in Table 76A, or a fragment thereof. The invention also includes a mutant or variant nucleic acid any of whose bases may be changed from the corresponding base shown in Table 76A while still encoding a protein that maintains its dual specificity phosphatase-like activities and physiological functions, or a fragment of such a nucleic acid. The invention further includes nucleic acids whose sequences are complementary to the sequence of Table 76A, including nucleic acid fragments that are complementary to any of the nucleic acids just described.

The invention additionally includes nucleic acids or nucleic acid fragments, or complements thereto, whose structures include chemical modifications. Such modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 9% of the bases may be so changed.

The novel protein of the invention includes the dual specificity phosphatase-like protein whose sequence is provided in Table 76B. The invention also includes a mutant or variant protein any of whose residues may be changed from the corresponding residue shown in Table 76B while still encoding a protein that maintains its dual specificity phosphatase-like activities and physiological functions, or a functional fragment thereof. In the mutant or variant protein, up to about 10% of the amino acid residues may be so changed.

These materials are further useful in the generation of antibodies that bind immunospecifically to the substances of the invention for use in therapeutic or diagnostic methods. These antibodies may be generated according to methods known in the art, using prediction from hydrophobicity charts, as described in the “Anti-NOVX Antibodies” section below.

NOV78

The disclosed NOV78 (alternatively referred to herein as CG56810-01) includes the 777 nucleotide sequence (SEQ ID NO:261) shown in Table 78A. A NOV78 ORF begins with a Kozak consensus ATG initiation codon at nucleotides 61-63 and ends with a stop codon at nucleotides 768-770. The disclosed NOV78 maps to human chromosome 2.

TABLE 78A
NOV78 Nucleotide Sequence

(SEQ ID NO:261)

TCACCTGAGCCTAGGAGTTCAAGATTGCAGTGGCCTATGATTGCATCATTGCACTCCAGCCTGGGTGACA
TCACCTGAGCCTAGGAGTTCAACATTGCAGTGGCCTATGATTGCATCATTGCACTCCAGCCTGGGTGACA
GAAAGAGACCCTGTCTCTGCATGATGATAATAATAATTAAAAGAGAGAGAGAGAGAGAGAAAACATATAG
TAGAAGGAGCACTTCTGGTGTGGGATTGAAGCAGTATTATCATTCAAGTGAGTCGCATTTAAATTTTTTA
CCTTTCTTGTTACCGATATCAACACCCCAGTCTCTTTTTAGGTACCCTAATTCATGGGATTTAAAACAAT
CATCTTTTTTCTTTCTCTTTAAGGTGACTCATATTCTTAATGTTGCATATGGAGTTGAAAATGCTTTCCT
CAGTGACTTTACATATAAGAGCATTTCTATATTGGATCTGCCTGAAACCAACATCCTGTCTTATTTTCCA
GAATGTTTTGAATTTATTGAAGAAGCAAAAAGAAAAGTGAGTTTTGTTTTGATCCATAGTTCTGCAGGAG
TGGTTCTTGTTCATTGTAATGCAGGCGTTTCCAGGGCTGCTGCAATTGTAATAGGTTTCCTGATGAATTC
TGAACAAACCTCATTTACCAGTGCTTTTTCTTTGGTGAAAAATGCAAGACCTTCCATATGTCCAAATTCT
GGCTTCATGGAGCAGCTTCGTACATATCAAGAGGGCAAAGAAAGCAATAAGTGTGACAGAATACAGCAGA
ACAGTTCATGAGTTGCATTGTAGCAGACAATGGACAACTGTAGTTTCTGAATTGACTTCTATAGCCATCT
TTTCCCT

A NOV78 polypeptide (SEQ ID NO:262) encoded by SEQ ID NO:261 is 224 amino acids in length and is presented using the one-letter amino acid code in Table 78B. The Psort profile for NOV78 predicts that this sequence has no signal peptide and is likely to be localized to the endoplasmic reticulum (membrane) with a certainty of 0.6400. In alternative embodiments, a NOV78 polypeptide is located to the plasma membrane with a certainty of 0.4960, or to the nucleus with a certainty of 0.2420.

TABLE 78B
NOV78 Polypeptide Sequence

| | |
|---|---|
| MIASLHSSLGDRKRPCLCMMIIIIKREREREKTYSRRSTSGVGLKQYYHSSESHLNFLPF | (SEQ ID NO:262) |
| LLPISTPQSLFRYPNSWDLKQSSFFFLFKVTHILNVAYGVENAFLSDFTYKSISILDLPE | |
| TNILSYFPECFEFIEEAKRKVSFVLIHSSAGVVLVHCNAGVSRAAAIVIGFLMNSEQTSF | |
| TSAFSLVKNARPSICPNSGFMEQLRTYQEGKESNKCDRIQENSS | |

A BLAST analysis of NOV78 was run against the proprietary PatP GENESEQ Protein Patent database. It was found, for example, that the amino acid sequence of NOV78 had high homology to other proteins as shown in Table 78C.

TABLE 78C
BLASTX results from PatP database for NOV78

| | | Smallest |
|---|---|---|
| | | Sum |
| | High | Probability |
| Sequences producing High-scoring Segment Pairs: | Score | P(N) |
| patp: AAB29109 Human cellular proliferative | 618 | 4.0e−60 |
| response protein | | |
| patp: AAB73224 Human phosphatase | 618 | 4.0e−60 |
| AI031656_h - Homo sapiens | | |
| patp: AAB73215 Murine phosphatase | 508 | 1.8e−48 |
| AA274457_m | | |
| patp: AAM42211 Human polypeptide | 290 | 2.3e−25 |
| patp: AAB94018 Human protein sequence | 201 | 6.2e−16 |

In a search of sequence databases, it was found, for example, that the nucleic acid sequence of this invention has 103 of 160 bases (64%) identical to a gb:GENBANK-ID:HS106C24|acc:Z83313.1 mRNA from Homo sapiens (Human DNA sequence from PAC 106C24, between markers DXS294 and DXS730 on chromosome X). The full amino acid sequence of the protein of the invention was found to have 71 of 172 amino acid residues (41%) identical to, and 99 of 172 amino acid residues (57%) similar to, the 203 amino acid residue ptnr:SPTREMBL-ACC:Q9NGL1 protein from Drosophila melanogaster (Fruit fly) (MAP KINASE PHOSPHATASE-1). NOV78 also has homology to the other proteins shown in the BLASTP data in Table 78D.

TABLE 78D
NOV78 BLASTP results

| Gene Index/ | | | Identity | Positive | |
|---|---|---|---|---|---|
| Identifier | Protein/Organism | Length (aa) | (%) | (%) | Expect |
| gi\|18146956\|dbj\| | protein phosphatase | 217 | 126/136 | 126/136 | 6e−65 |
| BAB82499.1\| | [Homo sapiens] | | (92) | (92) | |
| (AB038770) | | | | | |
| gi\|13277360\|ref\| | dual-specificity | 220 | 103/131 | 113/131 | 3e−54 |
| NP_077758.1\| | phosphatase | | (78) | (85) | |
| (NM_024438) | [Mus musculus] | | | | |
| gi\|12845353\|dbj\| | Dual specificity protein | 220 | 102/131 | 113/131 | 1e−53 |
| BAB26718.1\| | phosphatase containing | | (77) | (85) | |
| (AK010127) | protein [Mus musculus] | | | | |
| gi\|12858039\|dbj\| | Dual specificity protein | 162 | 103/131 | 113/131 | 1e−52 |
| BAB31181.1\| | phosphatase containing | | (78) | (85) | |
| (AK018369) | protein | | | | |
| gi\|18148911\|dbj\| | SKRP1 [Homo sapiens] | 166 | 74/74 | 74/74 | 7e−37 |
| BAB83499.1\| | | | (100) | (100) | |
| (AB063187) | | | | | |

This BLASTP data is displayed graphically in the ClustalW in Table 78E. A multiple sequence alignment is given, with the NOV78 protein being shown on line 1 in a ClustalW analysis comparing the protein of the invention with the related protein sequences shown in Table 78D.

[image]

[image]

Table 78F lists the domain description from DOMAIN analysis results against NOV78. This indicates that the NOV78 sequence has properties similar to those of other proteins known to contain this domain.

TABLE 78F
Domain Analysis of NOV78

gn1|Pfam|pfam00782, DSPc, Dual specificity phosphatase, catalytic domain.
Ser/Thr and Tyr protein phosphatases. The enzyme's tertiary fold is highly
similar to that of tyrosine-specific phosphatases, except for a “recognition”
region. SEQ ID NO:872
CD-Length = 139 residues, 80.6% aligned
Score = 112 bits (280), Expect = 2e−26

| | | | |
|---|---|---|---|
| NOV78: | 88 | FKVTHILNVAYGVENAFLSDFTYKSISILDLPETNILSYFPECFEFIEEAKRKVSFVLIH | 147 |
| | | +TH++NV V N+ S F Y I + D ET+I Y E EFIE+A++K | |
| Sbjct: | 26 | LGITHVINVTEEVPNSKNSGFLYLHIPVDDNHETDISPYLDEAVEFIEDARQK------- | 78 |
| NOV78: | 148 | SSACVVLVHCNAGVSRAAAIVIGFLMNSEQTSFTSAFSLVKNARPSICPNSOFMEQLRTY | 207 |
| | | G VLVHC AG+SR+A ++I +LM + S A+S VK RP I PN GF QL Y | |
| Sbjct: | 79 | --GGKVLVHCQAGISRSATLIIAYLMKTRNLSLNEAYSFVKERRPIISPNFGFKRQLIEY | P36 |
| NOV78: | 208 | Q 208 | |
| | | + | |
| Sbjct: | 137 | E 137 | |

Mitogen-activated protein (MAP) kinase phosphatases constitute a growing family of dual specificity phosphatases thought to play a role in the dephosphorylation and inactivation of MAP kinases and are therefore likely to be important in the regulation of diverse cellular processes such as proliferation, differentiation, and apoptosis. For this reason it has been suggested that MAP kinase phosphatases may be tumor suppressors.

NOV78 is predicted to be expressed in at least the following tissues: parathyroid gland, peripheral blood, whole organism. This information was derived by determining the tissue sources of the sequences that were included in the invention including but not limited to SeqCalling sources, public EST sources, literature sources, and/or RACE sources. Further expression data for NOV78 is provided in Example 2.

The NOV78 nucleic acids and proteins of are useful in potential therapeutic applications implicated in various pathological disorders described further herein, for example, brain disorders including epilepsy, eating disorders, schizophrenia, ADD, and cancer; heart disease; blood disorders, kidney disorders, liver diseases, inflammation and autoimmune disorders including Crohn's disease, IBD, allergies, rheumatoid and osteoarthritis, inflammatory skin disorders, allergies, blood disorders; psoriasis; colon-, ovarian-, testicular-, lymphatic-, brain-, and pancreatic cancers; leukemia AIDS; thalamus disorders; metabolic disorders including diabetes and obesity; lung diseases such as asthma, emphysema, cystic fibrosis, and cancer; pancreatic disorders including pancreatic insufficiency; and prostate disorders including prostate cancer and other diseases, disorders and conditions of the like. NOV78 nucleic acids encoding the MAP kinase-like protein of the invention, or fragments thereof, may further be useful in diagnostic applications, wherein the presence or amount of the nucleic acid or the protein are to be assessed.

The novel nucleic acid of the invention encoding a dual specificity phosphatase-like protein includes the nucleic acid whose sequence is provided in Table 78A, or a fragment thereof. The invention also includes a mutant or variant nucleic acid any of whose bases may be changed from the corresponding base shown in Table 78A while still encoding a protein that maintains its dual specificity phosphatase-like activities and physiological functions, or a fragment of such a nucleic acid. The invention further includes nucleic acids whose sequences are complementary to the sequence of Table 78A, including nucleic acid fragments that are complementary to any of the nucleic acids just described.

The invention additionally includes nucleic acids or nucleic acid fragments, or complements thereto, whose structures include chemical modifications. Such modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 36% of the bases may be so changed.

The novel protein of the invention includes the dual specificity phosphatase-like protein whose sequence is provided in Table 78B. The invention also includes a mutant or variant protein any of whose residues may be changed from the corresponding residue shown in Table 78B while still encoding a protein that maintains its dual specificity phosphatase-like activities and physiological functions, or a functional fragment thereof. In the mutant or variant protein, up to about 59% of the amino acid residues may be so changed.

These materials are further useful in the generation of antibodies that bind immunospecifically to the substances of the invention for use in therapeutic or diagnostic methods. These antibodies may be generated according to methods known in the art, using prediction from hydrophobicity charts, as described in the “Anti-NOVX Antibodies” section below.

NOV79

The disclosed NOV79 (alternatively referred to herein as CG56862-01) includes the 939 nucleotide sequence (SEQ ID NO:263) shown in Table 79A. A NOV79 ORF begins with a Kozak consensus ATG initiation codon at nucleotides 19-21 and ends with a stop codon at nucleotides 928-930. The disclosed NOV79 maps to human chromosome 20.

TABLE 79A
NOV79 Nucleotide Sequence

(SEQ ID NO:263)

TACAAGCAGGGTCTCCCTATGTTTCCCAGGCTGGTCTCAAACTCCTGGGCTCACACAGTCCTCCTGCCTT
GGCCTCCCAAAGTTCTGGGATTACAGACCCTGCAGGCGTCGGGCCTGGGCCGTCAGGGCAGCTGTGACCG
GATCGCTTCCCGGGCGGCGAGCTGGGGGTGCACCCGGACCGCCGCCCCCCGGATCATGGCCAATGGCATG
ACCAAGGTACTTCCTGGACTCTACCTCGGAAACTTCATTGGTCATCCCGCCAGCCAGATTGGCTCAAGCA
TCCTGTTTCTTTCAGATGCCAAAGACCTGGATCAGCTGGGCCGAAATAAGATCACACACATCATCTCTAT
CCATGAGTCACCCCAGCCTCTGCTGCAGGATATCACCTACCTTCGCATCCCGGTCGCTGATACCCCTGAG
GTACCCATGAAAAAGCACTTCAAAGAATGTATCAACTTCATCCACTGCTGCCGCCTTAATGGGGGGAACT
GCCTTGTGCACACCACGATTGTGACAGCGTATGTGATGACTGTGACGGGGCTAGGCTGGCGGGACGTGCT
TGAAGCCATCAAGGCCACCAGGCCCATCGCCAACCCCAACCCAGGCTTTAGGCAGCAGCTTGAAGAGTTT
GGCTGGGCCAGTTCCCAGAAGGTACAGCTTCGCCGGCAGCTGGAGGAGCGCTTCGGCGAGAGCCCCTTCC
GCGACGAGGAGGAGTTGCGCGCGCTGCTGCCGCTGTGCAAGCGCTGCCCGCAGGGCTCCGCGACCTCGGC
CTCCTCCGCCCGGCCGCACTCAGCAGCCTCCGAGGGAACCGTGCAGCGCCTGGTGCCGCGCACGCCCCGG
GAAGCCCACCGGCCGCTGCCGCTGCTGGCGCGCGTCAAGCAGACTTTCTCTTGCCTCCCCCGGTGTCTGT
CCCGCAAGGGCGGCAAGTGAGGATGCAGT

A NOV79 polypeptide (SEQ ID NO:264) encoded by SEQ ID NO:263 is 303 amino acids in length and is presented using the one-letter amino acid code in Table 79B. The Psort profile for NOV79 predicts that this sequence has a signal peptide and is likely to be localized to the cytoplasm with a certainty of 0.6500. In alternative embodiments, a NOV79 polypeptide is located to lysosomes with a certainty of 0.2216. The Signal P predicts a likely cleavage site for a NOV79 peptide is between positions 24 and 25, i.e., at the dash in the sequence VLG-LQ.

TABLE 79B
NOV79 Polypeptide Sequence

| | |
|---|---|
| MFPRLVSNSWAHTVLLPWPPKVLGLQTLQASGLGRQGSCDRIASRAASWGCTRTAAPGIM | (SEQ ID NO:264) |
| GNGMTKVLPGLYLGNFIGHPASQIGSSILFLSDAKDLDQLGRNKITHIISIHESPQPLLQ | |
| DITYLRIPVADTPEVPMKKHFKECINFIHCCRLNGGNCLVHTTIVTAYVMTVTGLGWRDV | |
| LEAIKATRPIANPNPGFRQQLEEFGWASSQKVQLRRQLEERFGESPFRDEEELRALLPLC | |
| KRCRQGSATSASSAGPHSAASEGTVQRLVPRTPREAHRPLPLLARVKQTFSCLPRCLSRK | |
| GGK | |

A BLAST analysis of NOV79 was run against the proprietary PatP GENESEQ Protein Patent database. It was found, for example, that the amino acid sequence of NOV79 had high homology to other proteins as shown in Table 79C.

TABLE 79C
BLASTX results from PatP database for NOV79

| | | Smallest |
|---|---|---|
| | | Sum |
| | High | Probability |
| Sequences producing High-scoring Segment Pairs: | Score | P(N) |
| patp: AAE04840 Human SGP008 phosphatase | 1298 | 3.5e−132 |
| polypeptide | | |
| patp: AAY68795 Amino acid sequence of a | 433 | 1.6e−40 |
| human protein | | |
| patp: AAB67167 Human dual-specificity | 433 | 1.6e−40 |
| phosphatase DSP-3 | | |
| patp: AAB66431 Human DSP-3 protein - | 433 | 1.6e−40 |
| Homo sapiens, 184 aa. | | |
| patp: AAB73216 Human phosphatase | 433 | 1.6e−40 |
| AA374753_h - Homo sapiens | | |

In a search of sequence databases, it was found, for example, that the nucleic acid sequence of this invention has 93 of 118 bases (78%) identical to a gb:GENBANK-ID:HUMFLNG6PD|acc:L44140.1 mRNA from Homo sapiens (chromosome X region from filamin (FLN) gene to glucose-6-phosphate dehydrogenase (G6PD). The full amino acid sequence of the protein of the invention was found to have 273 of 276 amino acid residues (98%) identical to, and 274 of 276 amino acid residues (99%) similar to, the 275 amino acid residue ptnr:TREMBLNEW-ACC:CAC10008 protein from Homo sapiens (Human) (BA243J16.6 (NOVEL PROTEIN)). NOV79 also has homology to the other proteins shown in the BLASTP data in Table 79D.

TABLE 79D
NOV79 BLASTP results

| Gene Index/ | | | Identity | Positive | |
|---|---|---|---|---|---|
| Identifier | Protein/Organism | Length (aa) | (%) | (%) | Expect |
| gi\|18104942\|ref\| | dual specificity | 243 | 241/245 | 242/245 | e−133 |
| NP_542178.1\| | phosphatase-like 15 | | (98) | (98) | |
| (NM_080611) | [Homo sapiens] | | | | |
| gi\|17458347\|ref\| | similar to bA243J16.6 | 235 | 226/252 | 227/252 | e−121 |
| XP_059288.1\| | (novel protein with a | | (89) | (89) | |
| (XM_059288) | dual specificity | | | | |
| | phosphatase, catalytic | | | | |
| | domain) (H. | | | | |
| | sapiens) [Homo sapiens] | | | | |
| gi\|9910432\|ref\| | mitogen-activated | 184 | 88/186 | 119/186 | 3e−44 |
| NP_064570.1\| | protein kinase | | (47) | (63) | |
| (NM_020185) | phosphatase x; homolog | | | | |
| | of mouse dual | | | | |
| | specificity phosphatase | | | | |
| | LMW-DSP2; JNK- | | | | |
| | stimulating phosphatase | | | | |
| | 1 [Homo sapiens] | | | | |
| gi\|13183069\|gb\| | dual specificity | 184 | 87/186 | 119/186 | 1e−43 |
| AAK15038.1\| | phosphatase TS-DSP2 [Mus | | (46) | (63) | |
| AF237619_1 | musculus] | | | | |
| (AF237619) | | | | | |
| gi\|14726046\|ref\| | mitogen-activated | 184 | 86/186 | 112/186 | 2e−41 |
| XP_046543.1\| | protein kinase | | (46) | (59) | |
| (XM_046543) | phosphatase x [Homo | | | | |
| | sapiens] | | | | |

This BLASTP data is displayed graphically in the ClustalW in Table 79E. A multiple sequence alignment is given, with the NOV79 protein being shown on line 1 in a ClustalW analysis comparing the protein of the invention with the related protein sequences shown in Table 79D.

[image]

[image]

Table 79F lists the domain description from DOMAIN analysis results against NOV79. This indicates that the NOV79 sequence has properties similar to those of other proteins known to contain this domain.

TABLE 79F
Domain Analysis of NOV79

gn1|Smart|smart00195, DSPc, Dual specificity phosphatase, catalytic domain
SEQ ID NO:873
CD-Length = 139 residues, 97.8% aligned
Score = 108 bits (271), Expect = 3e−25

| | | | |
|---|---|---|---|
| NOV79: | 63 | GMTKVLPGLYLGNFIGHPASQIGSSILFLSDAKDLOQLGRNKTTHIIS-IHESPQPLLQD | 121 |
| | | G +++LP LYLG++ SDA +L L + ITH+I+ E P | |
| Sbjct: | 1 | GPSEILPHLYLGSY---------------SDASNLALLKKLGITHVINVTEEVPNSNKSG | 45 |
| NOV79: | 122 | ITYLRIPVADTPEVPMKKHFKECINFIHCCRLNGGNCLVH--------TTIVTAYVMTVT | 173 |
| | | YL IPV D E + + E + FI GG LVH T++ AY+M | |
| Sbjct: | 46 | FLYLGIPVDDNTETKISPYLPEAVEFIEDAEKKGc4KVLVHCQAGVSRSATLIIAYLMKYR | 105 |
| NOV79: | 174 | GLGWRDVLEAIKATRPIANPNPGFRQQLEEF 204 | |
| | | + D + +K RPI +PN GF +QL E+ | |
| Sbjct: | 106 | NMSLNDAYDFVKERRPIISPNFGFLRQLIEY 136 | |

Mitogen-activated protein (MAP) kinase phosphatases constitute a growing family of dual specificity phosphatases thought to play a role in the dephosphorylation and inactivation of MAP kinases and are therefore likely to be important in the regulation of diverse cellular processes such as proliferation, differentiation, and apoptosis. For this reason it has been suggested that MAP kinase phosphatases may be tumor suppressors.

NOV79 is predicted to be expressed in at least the following tissues: brain, kidney, pancreas, testis, whole organism. This information was derived by determining the tissue sources of the sequences that were included in the invention including but not limited to SeqCalling sources, public EST sources, literature sources, and/or RACE sources. Further expression data for NOV79 is provided in Example 2.

The NOV79 nucleic acids and proteins of are useful in potential therapeutic applications implicated in various pathological disorders described further herein, for example, brain disorders including epilepsy, eating disorders, schizophrenia, ADD, and cancer; heart disease; blood disorders, kidney disorders, liver diseases, inflammation and autoimmune disorders including Crohn's disease, IBD, allergies, rheumatoid and osteoarthritis, inflammatory skin disorders, allergies, blood disorders; psoriasis; colon-, ovarian-, testicular-, lymphatic-, brain-, and pancreatic cancers; leukemia AIDS; thalamus disorders; metabolic disorders including diabetes and obesity; lung diseases such as asthma, emphysema, cystic fibrosis, and cancer; pancreatic disorders including pancreatic insufficiency; and prostate disorders including prostate cancer and other diseases, disorders and conditions of the like. The NOV79 nucleic acid encoding the phosphatase-like protein of the invention, or fragments thereof, may further be useful in diagnostic applications, wherein the presence or amount of the nucleic acid or the protein are to be assessed.

The novel nucleic acid of the invention encoding a dual specificity phosphatase-like protein includes the nucleic acid whose sequence is provided in Table 79A, or a fragment thereof. The invention also includes a mutant or variant nucleic acid any of whose bases may be changed from the corresponding base shown in Table 79A while still encoding a protein that maintains its dual specificity phosphatase-like activities and physiological functions, or a fragment of such a nucleic acid. The invention further includes nucleic acids whose sequences are complementary to the sequence of Table 79A, including nucleic acid fragments that are complementary to any of the nucleic acids just described.

The invention additionally includes nucleic acids or nucleic acid fragments, or complements thereto, whose structures include chemical modifications. Such modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 22% of the bases may be so changed.

The novel protein of the invention includes the dual specificity phosphatase-like protein whose sequence is provided in Table 79B. The invention also includes a mutant or variant protein any of whose residues may be changed from the corresponding residue shown in Table 79B while still encoding a protein that maintains its dual specificity phosphatase-like activities and physiological functions, or a functional fragment thereof. In the mutant or variant protein, up to about 2% of the amino acid residues may be so changed.

These materials are further useful in the generation of antibodies that bind immunospecifically to the substances of the invention for use in therapeutic or diagnostic methods. These antibodies may be generated according to methods known in the art, using prediction from hydrophobicity charts, as described in the “Anti-NOVX Antibodies” section below.

NOV80

The disclosed NOV80 (alternatively referred to herein as CG56882-01) includes the 2039 nucleotide sequence (SEQ ID NO:265) shown in Table 80A. A NOV80 ORF begins with a Kozak consensus ATG initiation codon at nucleotides 100-102 and ends with a TGA codon at nucleotides 1947-1949. The disclosed NOV80 maps to human chromosome 10.

TABLE 80A
NOV80 Nucleotide Sequence

(SEQ ID NO:265)

CATGTGCTAGGTTATTCCCAGTGCGAGGCCACACTTGGGCCGTCGGAGCAGCCCCTCCTCACTTCAGGGG
TCACCCTCCCCAAGACCCATTGCCCCATCATGGCCGGGGACCGGCTCCCCAGGAAGGTGATGGACGCCAA
GAAGCTGGCCAGCCTGCTGCGGGGCGGGCCTGGGGGGGGCCTGGTCATCGACAGTCACTCCTTCCTGGAG
TACAACAGCTGGCATGTGCTCAGCTCCGTCAACATCTGCTGCTCCAAGCTGGTGAAGTGGCGGTTGCAGA
AGGCCAAGGTGACCATTGTGGAGTTCATCCAGCCGGCCGCACGCAGCCAGGTGGAAGCCACTGAGCCACA
GGACGTGGTGGTCTATGACCAGAGCACGCGGGCCGCAGACAGCTTCCTCTCCATCCTGCTGAGCAAGCTG
GATGGCTGCTTCCACAGCGTGGCCGGCTGCTTCCACAGCATGGCCATCATCACGGGGAACTTCGCCACCT
TCTCCTCCTGCTTCCCCGACCTCTGCGAGCGCGAGCCTGCTGCCCTGCTACCCATCAGCCTCTCCCAGTC
CTGCCTGCTCGTGCCCAGCGTGGGCCTGACCCTCATCCTGCCTCACCTCTACCTGCGCTCGCAGGAAGAC
GTCCTGAACAAGGATCTGATGACGCAGAATGGAATAAGCTACGTCCTCTATGCCAGCAACTCCTGCCCCA
AGCCTGACTTCATCTACCAGAGCCACTTCTTGCGGGTCCCCATCAACGACAACTACTGTGAAAAGCTGCT
GCCCTGGCTGGACAAGTCCATCGAGTTCGTCGATAAAGCCAAGCTGTCCAGCTGCCAAGTCATCGTCCAC
CGTCTGGCCGGCATCTCCTGCTGTGCCACTATCGCCATCGCCTACATCATGAAGACCATGGGCATGTCCT
CCGAAGACGCCTACAGGTTTGTGAAGGACCAGCGCCCGTCCATCTCGCCCAACTTCAACTTCCTGGGCCA
GCTGCTGGAGGACCAGAGCAGCCCGAAGCTGCTGGCCGCCGTGCAGAACGACGCGGGCACCCCCTCAGGA
ATGCAGGAGCCTCCCCCCAGCCCTGCGGCCGGGGCCCCACTGCCATGGCTGCCACCACCTACCTCAGAGA
CCGCTGCCACCAGGAGTGCGGCTGCCAGGGAGGGCGGCCCGAGCGCAAGCAGGAAGCCCCCAACGCCCCC
CACGGCCACCAGCACGCTGCAGCAGGGCCTGCGCAGCCTGCGCCTCTCCTCGGACCACCTGCAGGACACC
AGCCGCCTCAAGCCCTCCTTCTCTCTGGACATCAAGTCGGCCTACGCCCCCAGCAGGCGGCCCGGCGGCC
CGGGCCCAGCGACCCCGGCGAGGCCCCGAAGCTCTCTGAAAGCTGGACAGCCAGTCAAGGCCATGTTGGG
CCTGCCCTCGCCCTGCCCGGACGCCGCGCCCGCCGCCCGGGCCCAGCGACCCCGGCGAGGCCCCGAAGCT
AGCCAGTCGGGGCCATGTTGGGCCCCTGCCCGGACGCCGCGCCCAGGCACGCCCACGGCCCGGCGCGCTA
CCCCGCGCGCGGCCTTAACTTCGGCTACGCGGCTGCCGGGCCCTGGCCAGCCGGCCACCCCCGGAGCCTG
GACGCCACCGCTCGACTCCCTGAACCGTCCTCGGTGCTTCAGCCCCGAGGGCGTGCAAGGGCCGGGCACG
GTGCTGTTTGCGCCCTTCGGCCGGGCGAACGCCCCGGAACCCAACGGCTGCAGCGACCTGCCACAACGGG
AGGCAGCAAGGGCTGAGCCCGGGACGGGTCAGACGAGCTGGCCCGACGAGCTGGCCCCGGATTCGCACTT
CAAGTGCTGCAGCTGCCAGATGGAGTTCGAGGAGGGCATGGTGGAGGGGCGCGCGCGCGGCGAGGAGCTG
GCCGCCCTCGGCAAGCAGGGGACCTTCTCGGGCAGCGTGGAGGTCATCGAGATGTCCTGACCCCTCCGCT
GCCCTCGGCTCCGCCGCCCGCAGCTGGGCAGTTATAAATATATATTATATATAATGCAAAGAAAGGCAAA
TGGTTTTAC

A NOV80 polypeptide (SEQ ID NO:266) encoded by SEQ ID NO:265 is 616 amino acids in length and is presented using the one-letter amino acid code in Table 80B. The Psort profile for NOV80 predicts that this sequence is a Type Ib membrane protein, has no signal peptide, and is likely to be localized at the plasma membrane with a certainty of 0.7000. In alternative embodiments, a NOV80 polypeptide is located to peroximsomal microbodies with a certainty of 0.3000, to the mitochondrial inner membrane with a certainty of 0.2143, or to the nucleus with a certainty of 0.3000.

TABLE 80B
NOV80 Polypeptide Sequence

| | |
|---|---|
| MAGDRLPRKVMDAKKLASLLRGCPGGGLVIDSHSFLEYNSWBVLSSVNICCSKLVKWRLQ | (SEQ ID NO:266) |
| KGKVTTVEFIQPAARSQVEATEPQDVVVYDQSTRAADSFLSILLSKLDGCFHSVAGCFHS | |
| MAIITGGFATFSSCFPDLCEGEPAALLPMSLSQSCLLVPSVGLTLILPHLYLGSQEDVLN | |
| KDLMTQNGISYVLYASNSCPKPDFIYQSNFLRVPINDNYCEKLLPWLDKSIEFVDKAKLS | |
| SCQVIVHRLAGISCCATIAIAYIMKTMGMSSEDAYRFVKRDQRPSISPNFNLGQLLEDQS | |
| SPKLLAAVQGDAGTPSGMQEPPPSPAAGAPLPWLPPPTSETAATRSAAAREGGPSAGRKP | |
| PAPPTATSTLQQGLRSLRLSSDHLQDTSRLKPSFSLDIKSAYAPSRRPGGPGPATPARPR | |
| SSLKAGQPVGANLCLPSPCPDAAPAARAQRPRRGPEASQSGPCWAPARTPRPGTPTARRA | |
| TPRAALTSATRLPGPGQPASPGAWTPPLDSLKRPRCFSPEGVQGPGRVLFAPFGRAGAPE | |
| PNGCSDLPRREAARAEPGTGQTSWPDELAPDSHFKCCSCQMEFEEGMVEGRARGEELAAL | |
| GKQGSFSGSVEVIEMS | |

A BLAST analysis of NOV80 was run against the proprietary PatP GENESEQ Protein Patent database. It was found, for example, that the amino acid sequence of NOV80 had high homology to other proteins as shown in Table 80C.

TABLE 80C
BLASTX results from PatP database for NOV80

| | | Smallest |
|---|---|---|
| | | Sum |
| | High | Probability |
| Sequences producing High-scoring Segment Pairs: | Score | P(N) |
| patp: AAW29150 Dual-specific murine thr-tyr | 1873 | 4.1e−193 |
| phosphatase | | |
| patp: AAE04834 Human SGP002 phosphatase | 950 | 6.6e−108 |
| polypeptide | | |
| patp: AAU09016 Human dual specificity | 950 | 6.6e−108 |
| phosphatase 21117 | | |
| patp: AAM25744 Human protein sequence | 955 | 7.8e−96 |
| patp: AAB20325 Human protein phosphatase and | 949 | 3.4e−95 |
| kinase protein | | |

In a search of sequence databases, it was found, for example, that the nucleic acid sequence of this invention has 1587 of 1930 bases (82%) identical to a gb:GENBANK-ID:HSU27193|acc:U27193.1 mRNA from Homo sapiens (Human protein-tyrosine phosphatase mRNA). The full amino acid sequence of the protein of the invention was found to have 489 of 625 amino acid residues (78%) identical to, and 514 of 625 amino acid residues (82%) similar to, the 625 amino acid residue ptnr:SWISSNEW-ACC:Q13202 protein from Homo sapiens (Human) (DUAL SPECIFICITY PROTEIN PHOSPHATASE 8 (EC 3.1.3.48) (EC 3.1.3.16) (DUAL SPECIFICITY PROTEIN PHOSPHATASE HVH-5)). (NOV80 also has homology to the other proteins shown in the BLASTP data in Table 80D.

TABLE 80D
NOV80 BLASTP results

| Gene Index/ | | | Identity | Positive | |
|---|---|---|---|---|---|
| Identifier | Protein/Organism | Length (aa) | (%) | (%) | Expect |
| gi\|4758212\|ref\| | dual specificity | 625 | 480/632 | 506/632 | 0.0 |
| NP_004411.1\| | phosphatase 8; H1 | | (75) | (79) | |
| (NM_004420) | phosphatase, vaccinia | | | | |
| | virus homolog; protein | | | | |
| | tyrosine phosphatase; | | | | |
| | serine/threonine | | | | |
| | specific protein | | | | |
| | phosphatase | | | | |
| | [Homo sapiens] | | | | |
| gi\|6679156\|ref\| | neuronal | 663 | 450/682 | 476/682 | 0.0 |
| NP_032774.1\| | tyrosine/threonine | | (65) | (68) | |
| (NM_008748) | phosphatase 1 | | | | |
| | [Mus musculus] | | | | |
| gi\|17471343\|ref\| | similar to dual | 461 | 324/422 | 333/422 | e−140 |
| XP_061101.1\| | specificity phosphatase | | (76) | (78) | |
| (XM_061101) | 8; H1 phosphatase, | | | | |
| | vaccinia virus homolog; | | | | |
| | protein | | | | |
| | tyrosinephosphatase; | | | | |
| | serine/threonine | | | | |
| | specific protein | | | | |
| | phosphatase (H. sapiens) | | | | |
| | [Homo sapiens] | | | | |
| gi\|13639013\|ref\| | dual specificity | 501 | 343/473 | 363/473 | e−131 |
| XP_012007.2\| | phosphatase 8 | | (72) | (76) | |
| (XM_012007) | [Homo sapiens] | | | | |
| gi\|12697945\|dbj\| | KIAA1700 protein | 690 | 270/668 | 373/668 | e−103 |
| BAB21791.1\| | [Homo sapiens] | | (40) | (55) | |
| (AB051487) | | | | | |

This BLASTP data is displayed graphically in the ClustalW in Table 80E. A multiple sequence alignment is given, with the NOV80 protein being shown on line 1 in a ClustalW analysis comparing the protein of the invention with the related protein sequences shown in Table 80D.

[image]

[image]

[image]

[image]

Table 80F lists the domain description from DOMAIN analysis results against NOV80. This indicates that the NOV80 sequence has properties similar to those of other proteins known to contain this domain.

TABLE 80F
Domain Analysis of NOV80

gn1|Smart|smart00195, DSPc, Dual specificity phosphatase, catalytic domain
SEQ ID NO:873
CD-Length = 139 residues, 97.1% aligned
Score = 154 bits (388), Expect = 2e−38

| | | | |
|---|---|---|---|
| NOV80: | 162 | GLTLILPHLYLGSQEDVLNKDLMTQNGISYVLYASNSCPKPDFIYQSHFLRVPINDNYCE | 221 |
| | | G + ILPHLYLGS D N L+ +GI++V+ + P +L +P++DN | |
| Sbjct: | 1 | GPSEILPHLYLGSYSDASNLALLKKLGITHVINVTEEVPN-SNKSGFLYLGIPVDDNTET | 59 |
| NOV80: | 222 | KLLPWLDKSIEFVDKAKLSSCQVIVHRLAGISCCATIAIAYIMKTMGMSSEDAYRFVKDQ | 281 |
| | | K+ P+L +++EF++A+ +V+VH AG+S AT+ IAY+MK MS DAY FVK++ | |
| Sbjct: | 60 | KISPYLPEAVEFIEDAEKKGGKVLVHCQAGVSRSATLIIAYLMKYRNMSLNDAYDFVKER | 119 |
| NOV80: | 282 | RPSISPNFNFLGQLLE 297 | |
| | | RP ISPNF FL QL+E | |
| Sbjct: | 120 | RPIISPNFGFLRQLIE 135 | |

Mitogen-activated protein (MAP) kinase phosphatases constitute a growing family of dual specificity phosphatases thought to play a role in the dephosphorylation and inactivation of MAP kinases and are therefore likely to be important in the regulation of diverse cellular processes such as proliferation, differentiation, and apoptosis. For this reason it has been suggested that MAP kinase phosphatases may be tumor suppressors.

NOV80 is predicted to be expressed in at least the following tissues: kidney. This information was derived by determining the tissue sources of the sequences that were included in the invention including but not limited to SeqCalling sources, public EST sources, literature sources, and/or RACE sources. Further expression data for NOV80 is provided in Example 2.

The NOV80 nucleic acids and proteins of are useful in potential therapeutic applications implicated in various pathological disorders described further herein, for example, brain disorders including epilepsy, eating disorders, schizophrenia, ADD, and cancer; heart disease; blood disorders, kidney disorders, liver diseases, inflammation and autoimmune disorders including Crohn's disease, IBD, allergies, rheumatoid and osteoarthritis, inflammatory skin disorders, allergies, blood disorders; psoriasis; colon-, ovarian-, testicular-, lymphatic-, brain-, and pancreatic cancers; leukemia AIDS; thalamus disorders; metabolic disorders including diabetes and obesity; lung diseases such as asthma, emphysema, cystic fibrosis, and cancer; pancreatic disorders including pancreatic insufficiency; and prostate disorders including prostate cancer and other diseases, disorders and conditions of the like. The NOV80 nucleic acid encoding the phosphatase-like protein of the invention, or fragments thereof, may further be useful in diagnostic applications, wherein the presence or amount of the nucleic acid or the protein are to be assessed.

The novel nucleic acid of the invention encoding a dual specificity phosphatase-like protein includes the nucleic acid whose sequence is provided in Table 80A, or a fragment thereof. The invention also includes a mutant or variant nucleic acid any of whose bases may be changed from the corresponding base shown in Table 80A while still encoding a protein that maintains its dual specificity phosphatase-like activities and physiological functions, or a fragment of such a nucleic acid. The invention further includes nucleic acids whose sequences are complementary to the sequence of Table 80A, including nucleic acid fragments that are complementary to any of the nucleic acids just described.

The invention additionally includes nucleic acids or nucleic acid fragments, or complements thereto, whose structures include chemical modifications. Such modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 18% of the bases may be so changed.

The novel protein of the invention includes the dual specificity phosphatase-like protein whose sequence is provided in Table 80B. The invention also includes a mutant or variant protein any of whose residues may be changed from the corresponding residue shown in Table 80B while still encoding a protein that maintains its dual specificity phosphatase-like activities and physiological functions, or a functional fragment thereof. In the mutant or variant protein, up to about 22% of the amino acid residues may be so changed.

These materials are further useful in the generation of antibodies that bind immunospecifically to the substances of the invention for use in therapeutic or diagnostic methods. These antibodies may be generated according to methods known in the art, using prediction from hydrophobicity charts, as described in the “Anti-NOVX Antibodies” section below.

NOV81

NOV81 includes two galactosyltransferase-like proteins, designated herein as NOV81a and NOV81b.

NOV81a

The disclosed NOV81a (alternatively referred to herein as CG56283-01) includes the 1247 nucleotide sequence (SEQ ID NO:267) shown in Table 81A. A NOV81a ORF begins with a Kozak consensus ATG initiation codon at nucleotides 33-35 and ends with a stop codon at nucleotides 1224-1226. The disclosed NOV81a maps to human chromosome 19.

TABLE 81A
NOV81a Nucleotide Sequence

(SEQ ID NO:267)

CTCCCGCGGCCGCCCCTTCCCTGGGCCGCGTCATGCGCTGCCCCAAGTGCCTTCTCTGCCTGTCAGCACT
GCTCACACTCCTGGGCCTCAAAGTGTACATCGAATGGACATCCGAGTCCCGGCTCACCAAGGCCTACCCC
AGCCCTCGGGGCACCCCGCCAAGCCCCACGCCAGCCAACCCTGAGCCCACCCTACCTGCCAACCTCTCCA
CCCGCCTGGGCCAGACTATCCCGCTGCCCTTTGCTTACTGGAACCAGCAGCAGTGGCGGCTGGGGTCCCT
GCCCAGTGGGGACAGCACTGAAACGGGGGGCTGCCAGGCTTGGGGGGCCGCCGCCGCCACCGAGATCCCT
GACTTCGCCTCCTACCCCAAGGACCTCCQCCGCTTCTTGCTGTCAGCAGCCTGCCGGAGCTTCCCACAGT
GGCTGCCTGGACGTCGTCGCGGCCAAGTCTCCAGCTGCTCAGATACTGATGTCCCCTACCTGCTGTTGGC
CGTCAAGTCAGAACCAGGGCGCTTTGCAGAACGACAGGCCGTGAGAGAGACGTGGGGCAGTCCAGCTCCA
GGGATCCGGCTGCTCTTCCTGCTAGGGTCTCCCGTAGGTGAGGCGGGGCCTGACCTAGACTCACTAGTGG
CATGGGAGAGCCGTCGCTACAGTGACCTGCTGCTCTGGGACTTCCTCGACGTCCCATTCAACCAGACGCT
CAAAGACCTGCTGCTGCTGGCCTGGCTGGGCCGCCACTGCCCCACCGTGAGTTTTGTCTTGCGAGCTCAG
GACGATGCCTTTGTACACACCCCTGCCCTGCTCGCTCACCTGCGGGCCCTGCCACCTGCCTCGGCCCGAA
GCCTCTACCTGGGTGAGGTCTTTACCCAGGCCATCCCTCTCCGGAAGCCAGGAGGACCCTTCTATGTGCC
CGAGTCCTTCTTCGAAGGTGGCTACCCAGCCTATGCAAGCGGGGGTGGCTACGTCATTGCCGGGCGCCTG
CCACCCTGGCTGCTGCGGGCGGCAGCCCGTGTGGCACCCTTCCCCTTTGAGGACGTCTACACTGGCCTTT
GCATCCGAGCCCTGGGCCTGGTGCCCCAGGCCCACCCAGGCTTCCTCACAGCCTGGCCAGCAGACCGCAC
TGCGGACCACTGTCCTTTCCGCAACCTGCTGCTGCTACGGCCCCTGGGCCCCCAGCCCAGCATTCGGCTC
TGGAAACAACTGCAAGACCCAAGGCTCCAGTGCTGACTCTCATTGGGGAGGGCGGAG

A NOV81a polypeptide (SEQ ID NO:268) encoded by SEQ ID NO:267 is 397 amino acids in length and is presented using the one-letter amino acid code in Table 81B. The Psort profile for NOV81a predicts that this sequence has a signal peptide and is likely to be localized to lysosomes with a certainty of 0.8650, or to the outside of the cell with a certainty of 0.8191. The Signal P predicts a likely cleavage site for a NOV81a peptide is between positions 34 and 35, i.e., at the dash in the sequence SKA-YP.

TABLE 81B
NOV81a Polypeptide Sequence

| | |
|---|---|
| MRCPKCLLCLSALLTLLGLKVYIEWTSESRLSKAYPSPRGTPPSPTPANPEPTLPANLST | (SEQ ID NO:268) |
| RLGQTIPLPFAYWNQQQWRLGSLPSGDSTETGGCQAWGAAAATEIPDFASYPKDLRRFLL | |
| SAACRSFPQWLPGGGGGQVSSCSDTDVPYLLLAVKSEPGRFAERQAVRETWGSPAPGIRL | |
| LFLLGSPVGEAGPDLDSLVAWESRRYSDLLLWDFLDVPFNQTLKDLLLLAWLGRHCPTVS | |
| FVLRAQDDAFVHTPALLAHLRALPPASARSLYLGEVFTQANPLRKPGGPFYVPESFFEGG | |
| YPAYASGGGYVIAGRLAPWLLRAAARVAPFPFEDVYTGLCIRALGLVPQAHPGFLTAWPA | |
| DRTADHCAFRNLLLVRPLGPQASIRLWKQLQDPRLQC | |

The disclosed NOV81b (alternatively referred to herein as CG56283-02) includes the 1368 nucleotide sequence (SEQ ID NO:269) shown in Table 81C. A SEC2 ORF begins with a Kozak consensus ATG initiation codon at nucleotides 4-6 and ends with a TGA codon at nucleotides 1195-1197. In Table 81C, putative untranslated regions are indicated by underlining, and the stop and start codons are indicated in bold. The disclosed NOV81b maps to human chromosomes 19.

TABLE 81C
NOV81b Nucleotide Sequence

| | |
|---|---|
| GTCATGCGCTGCCCCAAGTGCCTTCTCTGCCTGTCAGCACTGCTCACACTCCTGGCCCTC | (SEQ ID NO:269) |
| AAAGTGTACATCGAGTGGACATCCGAGTCCCGGCTCAGCAAGGCCTACCCCAGCCCTCGG | |
| GGCACCCCGCCAAGCCCCACGCCAGCCAACCCTGAGCCCACCCTACCTGCCAACCTCTCC | |
| ACCCGCCTGGGCCAGACTATCCCGCTGCCCTTTGCTTACTGGAACCAGCAGCAGTGGCGG | |
| CTGGGGTCCCTGCCCAGTGGGGACAGCACTGAAACGGGGGGCTGCCAGGCTTGGGGGGCC | |
| GCCGCCGCCACCGAGATCCCTGACTTCGCCTCCCACCCCAAGGACCTCCGCCGCTTCTTG | |
| CTGTCAGCAGCCTGCCGGAGCTTCCCACAGTGGCTGCCTGGAGGTGGTGGCAGCCAAGTC | |
| TCCAGCTGCTCAGATACTGATGTCCCCTACCTGCTGTTGGCCGTCAAGTCAGAACCAGGG | |
| CGCTTTGCAGAACGACAGGCCGTGAGAGAGACGTGGGGCAGTCCAGCTCCAGGGATCCGG | |
| CTGCTCTTCCTGCTAGGGTCTCCGGTGGGTGAGGCGGGGCCTGACCTAGACTCACTAGTG | |
| GCCTGGGACACCCGTCGCTACAGTGACCTGCTGCTCTGGGACTTCCTCGACGTCCCATTC | |
| AACCAGACGCTCAAAGACCTGCTGCTGCTGGCCTGGCTGGGCCGCCACTGCCCCACCGTG | |
| AGTTTTGTCTTGCGAGCTCAGGACGATGCCTTTGTACACACCCCTGCCCTGCTGGCTCAC | |
| CTGCGGGCCCTGCCACCTGCCTCGGCCCGAAGCCTCTACCTGGGTGAGGTCTTTACCCAG | |
| GCCATGCCTCTCCGGAAGCCAGGAGGACCCTTCTATGTGCCCGAGTcCTTCTTCCAAGGT | |
| GGCTACCCAGCCTATGCAAGCGGGGGTGGCTACGTCATTGCCGGGCGCCTGGCACCCTGG | |
| CTGCTGCGGGCGGCAGCCCGTGTGGCACCCTTCCCCTTTGAGGACGTCTACACTGGCCTT | |
| TGCATCCCAGCCCTGGGCCTGGTGCCCCACGCCCACCCAGGCTTCCTCACAGCCTGGCCA | |
| GCAGACCGCACTGCGGACCACTGTGCTTTCCGCAACCTGCTGCTGGTACGGCCCCTGGGC | |
| CCCCAGGCCAGCATTCGGCTCTGGAAACAACTGCAAGACCCAAGGCTCCAGTGCTGACTC | |
| TCATTGGGGAGGGCGGACGTGCTGACCTGGCCCCGGCCCTGGCCTGGGCCTCTGGGGCCG | |
| GCCCCTGGCTCAGCCCCTCCTTCCAGGTCTTGATGGGAGGGAGGAGGGCCCAGAAGCTGG | |
| ACAACTTAAGCCACTCCTTGGCCTCCCCCAGCCAGGTGAGTGAGCTAT | |

A NOV81b polypeptide (SEQ ID NO:270) encoded by SEQ ID NO:269 is 397 amino acids in length and is presented using the one-letter amino acid code in Table 81D. The Psort profile for NOV81b predicts that this sequence has a signal peptide and is likely to be localized to lysosomes with a certainty of 0.8650, to the exterior of the cell with a certainty of 0.8190. The Signal P predicts a likely cleavage site for a NOV81b peptide is between positions 34 and 35, i.e., at the dash in the sequence SAK-YP.

TABLE 81D
NOV81b Polypeptide Sequence

| | |
|---|---|
| MRCPKCLLCLSALLTLLGLKVYIEWTSESRLSKAYPSPRGTPPSPTPANPEPTLPANLST | (SEQ ID NO:270) |
| RLCQTIPLPFAYWNQQQWRLGSLPSGDSTETGCCQAWCAAAATEIPDFASHPKDLRRFLL | |
| SAACRSFPQWLPCGGCSQVSSCSDTDVPYLLLAVKSEPGRFAERQAVRETWCSPAPCIRL | |
| LFLLGSPVGEAGPDLDSLVAWESRRYSDLLLWDFLDVPFNQTLKDLLLLAWLGRHCPTVS | |
| FVLRAQDDAFVHTPALLAHLRALPPASARSLYLGEVFTQAMPLRKPGGPFYVPESFFEGG | |
| YPAYASCGGYVIAGRLAPWLLRAAARVAPFPFEDVYTGLCIRALGLVPQAHPGFLTAWPA | |
| DRTADHCAFRNLLLVRPLGPQASIRLWKQLQDPRLQC | |

A BLAST analysis of NOV81 was run against the proprietary PatP GENESEQ Protein Patent database. It was found, for example, that the amino acid sequence of NOV81 had high homology to other proteins as shown in Table 81E.

TABLE 81E
BLASTX results from PatP database for NOV81

| | | Smallest |
|---|---|---|
| | | Sum |
| | High | Probability |
| Sequences producing High-scoring Segment Pairs: | Score | P(N) |
| patp: AAB03619 Human beta-1,3- | 2130 | 2.4e−220 |
| galactosyltransferase Znssp2 | | |
| patp: AAB03620 Murine beta-1,3- | 1585 | 1.4e−162 |
| galactosyltransferase Znssp | | |
| patp: AAM41987 Human polypeptide | 1528 | 1.5e−156 |
| patp: AAE05767 Human secreted protein (SECP) | 641 | 1.5e−62 |
| patp: AAM40201 Human polypeptide | 641 | 1.5e−62 |

In a search of sequence databases, it was found, for example, that the nucleic acid sequence of this invention has 647 of 1088 bases (59%) identical to a gb:GENBANK-ID:AP001754|acc:AP001754.1 mRNA from Homo sapiens (genomic DNA, chromosome 21q, section 98/105). The full amino acid sequence of the protein of the invention was found to have 127 of 343 amino acid residues (37%) identical to, and 194 of 343 amino acid residues (56%) similar to, the 397 amino acid residue ptnr:TREMBLNEW-ACC:AAD09763 protein from Mus musculus (Mouse) (BETA-1,3-N-ACETYLGLUCOSAMINYLTRANSFERASE (EC 2.4.1.149)). NOV81 also has homology to the other proteins shown in the BLASTP data in Table 81F.

TABLE 81F
NOV81 BLASTP results

| Gene Index/ | | Length | Identity | Positive | |
|---|---|---|---|---|---|
| Identifier | Protein/Organism | (aa) | (%) | (%) | Expect |
| gi\|14290592\|gb\| | beta-1,3-N- | 397 | 126/350 | 192/350 | 4e−58 |
| AAH09075.1\| | acetylglucosaminyltransferase | | (36) | (54) | |
| AAH09075 | 1 [Mus musculus] | | | | |
| (BC009075) | | | | | |
| gi\|16973463\|gb\| | beta-3- | 406 | 128/346 | 186/346 | 1e−57 |
| AAL32299.1\| | galactosyltransferase | | (36) | (52) | |
| AF321831_1 | [Danio rerio] | | | | |
| (AF321831) | | | | | |
| gi\|9938024\|ref\| | UDP-GlcNAc: betaGal beta- | 397 | 125/350 | 141/350 | 2e−57 |
| NP_058584.2\| | 1,3-N- | | (35) | (53) | |
| (NM_016888) | acetylglucosaminyltransferase | | | | |
| | 1; beta-1,3-N- | | | | |
| | acetylglucosaminyltransferase; | | | | |
| | beta-1,3-N- | | | | |
| | acetylglucosaminyltransferase | | | | |
| | 1; UDP- | | | | |
| | Gal: betaGlcNAc beta 1,3- | | | | |
| | galactosyltransferase, | | | | |
| | polypeptide 6 | | | | |
| | [Mus musculus] | | | | |
| gi\|9845238\|ref\| | beta-1,3-N- | 397 | 121/351 | 187/351 | 6e−56 |
| NP_006568.2\| | acetylglucosaminyltransferase | | (34) | (52) | |
| (NM_006577) | bGnT-1; beta3gal- | | | | |
| | T5 gene; beta-1,3-N- | | | | |
| | acetylglucosaminyltransferase | | | | |
| | bGnT-2 | | | | |
| | [Homo sapiens] | | | | |
| gi\|9664889\|gb\| | beta | 393 | 121/351 | 187/351 | 6e−56 |
| AAF97254.1\|AF288209_1 | galactosyltransferase | | (34) | (52) | |
| (AF288209) | bGalT7 [Homo sapiens] | | | | |

This BLASTP data is displayed graphically in the ClustalW in Table 81G. A multiple sequence alignment is given, with the NOV81 protein being shown on line 1 in a ClustalW analysis comparing the protein of the invention with the related protein sequences shown in Table 81F.

[image]

[image]

Table 81H lists the domain description from DOMAIN analysis results against NOV81. This indicates that the NOV81 sequence has properties similar to those of other proteins known to contain this domain.

TABLE 81E
Domain Analysis of NOV81

gn1|Pfam|pfam01762, Galactosyl_T, Galactosyltransferase. This family includes
the galactosyltransferases UDP-galactose:2-acetamido-2-deoxy-D-glucose3beta-
galactosyltransferase and UDP-Gal:beta-GlcNAc beta 1,3 -galactosyltranferase.
Specific galactosyltransferases transfer galactose to GlcNAc terminal chains
in the synthesis of the lacto-series oligosaccharides types 1 and 2. SEQ ID NO: 874
CD-Length = 195 residues, 98.5% aligned
Score = 77.4 bits (189), Expect = 1e−15

| | | | |
|---|---|---|---|
| NOV81: | 162 | AERQAVRETWGSP--APGIRL--LFLLGSPVGEAOPDLDSLVAWESRRYSDLLLWDFLDV | 217 |
| | | A R A+R+TW + +G R+ LFL+G + L LV E+R Y D+++ D D | |
| Sbjct: | 1 | ARRNAI RKTWMNQNNSRGGRIKSLFLVG--LAALDCKLKKLVMEEARLYGDIIVVDLEDS | 58 |
| NOV81: | 218 | PFNQTLKDLLLLAWLGRHCPTVSFVLRAQDDAFHTPALLAHLRALPPASARSLYLGEVF | 277 |
| | | N TLK L +L ++ CP + + DD FV+ LL+ L + + G + | |
| Sbjct: | 59 | YLNLTLKTLTILLYVVSKCPNAKLIGKIDDDVFVNPDNLLSLLEREyIDpSpLsFyGyII 118 | |
| NOV81: | 278 | TQAMPLRKPGGPFYVPESFF-EGGYPAYASGGGYVIAGRLAPWLLRAAARVAPFPFEDVY | 336 |
| | | P+R +YVP + + YP Y SG Y+++ AP +L+A+ EDV | 336 |
| Sbjct: | 119 | KNGEPVRTKKSKWYVPPTAYPCSNYPPYLSGPFYILSRDAAPLILKASKHRRFIKIEDVL | 178 |
| NOV81: | 337 | -TCLCIRALCLVPQ 349 | |
| | | TG+ LG+ | |
| Sbjct: | 179 | ITGILALDLGISRI 192 | |

There are 2 known types of carbohydrate chains in the lacto series of oligosaccharides: type 1 chains, which contain the Gal(beta-1-3)GlcNAc linkage, and type 2 chains, which contain the topoisomer Gal(beta-1-4)GlcNAc. The biosynthesis of both types of chains is catalyzed by specific galactosyltransferases (GalTs), which transfer galactose (Gal) to N-acetylglucosamine (GlcNAc)-terminating chains. Beta-4-GalT enzymes (e.g., GGTB2; are the galactosyltransferases responsible for type 2 chain biosynthesis, while beta-3-GalTs are the type 1 elongating enzymes.

Kolbinger et al. (1998) searched an expressed sequence tag (EST) database with the amino acid sequence of a human beta-3-GalT, which they called beta-3-GalT1, and identified human brain cDNAs encoding a novel beta-3-GalT, which they named beta-3-GalT2. The deduced 422-amino acid beta-3-GalT2 protein has a predicted type II transmembrane topology with 5 potential N-glycosylation sites, and a predicted molecular mass of 49,202 Da. Beta-3-GalT2 shares 46% amino acid identity with beta-3-GalT1, but has a 17-amino acid extension at the carboxy terminus and longer cytoplasmic and stem regions. Beta-3-GalT2 directed the synthesis of type 1 chains in mammalian cells and transferred Gal to GlcNAc- and Gal-terminating acceptors in enzymatic assays.

Northern blot analysis demonstrated strong expression of a 3.5-kb beta-3-GalT2 transcript, and weaker expression of a 2.8-kb transcript, in heart and brain. Amado et al. (1998) stated that a human beta-3-galactosyltransferase gene, called beta-3-GalT1 by them, was isolated from a melanoma cell line using a transfection-cloning strategy. Beta-3-GalT1 is a predicted 326-amino acid protein. By carrying out a BLAST search of an EST database with the beta-3-GalT1 coding sequence, Amado et al. (1998) identified cDNAs encoding 3 other beta-3-galactosyltransferases, beta-3-GalT2, beta-3-GalT3, and beta-3-GalT4. The sequences of the 4 predicted proteins share 29 to 42% identity and have several conserved short sequence motifs. All 4 appear to be evolutionarily related, since their coding regions are contained in a single exon.

Using an insect cell expression system, Amado et al. (1998) showed that beta-3-GalT1 and beta-3-GalT2 are UDP-galactose:beta-N-acetyl-glucosamine beta-1,3 galactosyltransferases with similar kinetic properties. Northern blot analysis revealed that beta-3-GalT1 is expressed as a 6.5-kb mRNA exclusively in brain. Hennet et al. (1998) identified a mouse beta-3-GalT1 homolog, designated beta-3-GalT1, and found that the coding region was contained in a single exon.

NOV81 is predicted to be expressed in at least the following tissues: colon, blood, and lymphocyte. This information was derived by determining the tissue sources of the sequences that were included in the invention including but not limited to SeqCalling sources, public EST sources, literature sources, and/or RACE sources. Further expression data for NOV81 is provided in Example 2.

The NOV81 nucleic acids and proteins of are useful in potential therapeutic applications implicated in various pathological disorders described further herein, for example, hemophilia, hypercoagulation, idiopathic thrombocytopenic purpura, autoimmume disease, allergies, immunodeficiencies, transplantation, graft versus host disease (GVHD), lymphaedema, anemia, ataxia-telangiectasia, autoimmume disease,immunodeficiencies, Hirschsprung's disease, Crohn's Disease, appendicitis as well as other diseases, disorders and conditions. NOV81 nucleic acids encoding the galactosyltransferase-like protein of the invention, or fragments thereof, may further be useful in diagnostic applications, wherein the presence or amount of the nucleic acid or the protein are to be assessed.

The novel nucleic acid of the invention encoding a beta-1,3-galactosyltransferase-like protein includes the nucleic acid whose sequence is provided in Table 81A or 81C, or a fragment thereof. The invention also includes a mutant or variant nucleic acid any of whose bases may be changed from the corresponding base shown in Table 81A or 81C while still encoding a protein that maintains its beta-1,3-galactosyltransferase-like activities and physiological functions, or a fragment of such a nucleic acid. The invention further includes nucleic acids whose sequences are complementary to the sequence of Table 81A or 81C, including nucleic acid fragments that are complementary to any of the nucleic acids just described.

The invention additionally includes nucleic acids or nucleic acid fragments, or complements thereto, whose structures include chemical modifications. Such modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 41% of the bases may be so changed.

The novel protein of the invention includes the beta-1,3-galactosyltransferase-like protein whose sequence is provided in Table 81B or 81D. The invention also includes a mutant or variant protein any of whose residues may be changed from the corresponding residue shown in Table 81B or 81D while still encoding a protein that maintains its beta-1,3-galactosyltransferase-like activities and physiological functions, or a functional fragment thereof. In the mutant or variant protein, up to about 63% of the amino acid residues may be so changed.

These materials are further useful in the generation of antibodies that bind immunospecifically to the substances of the invention for use in therapeutic or diagnostic methods. These antibodies may be generated according to methods known in the art, using prediction from hydrophobicity charts, as described in the “Anti-NOVX Antibodies” section below.

NOV82

The disclosed NOV82 (alternatively referred to herein as CG56983-01) includes the 348 nucleotide sequence (SEQ ID NO:271) shown in Table 82A. A NOV82 ORF begins with a Kozak consensus ATG initiation codon at nucleotides 9-11 and ends with a stop codon at nucleotides 321-322. The disclosed NOV82 maps to human chromosome X.

TABLE 82A
NOV82 Nucleotide Sequence

(SEQ ID NO:271)

CTATCCCTATGGTGTCGGTGTGCAGGCCGTGGCCTGCTGTGGCCATAGCACTTCTGGCTCTCCTGGTCTG
CCTGGGGGCGCTGGTCGACACCTGCCCCATCAAACCCGAGGCTCCTGGCGAAGACGAGTCCCTGGAGGAG
CTGAGCCACTATTATCCTTCCCTGTGCCACTACCTCAACGTGGTCACCAGACAGTTAATTTCAGAGAGAA
ACCTACCAGACACCATTGTGTCCAAGGAAGTATTTTTCACAAGCACAAAGGAAAGACCTGTGAGGACACA
GAAGGAAGGTTGCCATCTGACCGCCAAGGAGAGAAGCCTCTGAAAAAACCAAACCTGCTGGCACCTG

A NOV82 polypeptide (SEQ ID NO:272) encoded by SEQ ID NO:271 is 104 amino acids in length and is presented using the one-letter amino acid code in Table 82B. The Psort profile for NOV82 predicts that this sequence has a signal peptide and is likely to be localized at the exterior of the cell with a certainty of 0.8200. In alternative embodiments, a NOV82 polypeptide is located to the endoplasmic reticulum (membrane) with a certainty of 0.1000. The Signal P predicts a likely cleavage site for a NOV82 peptide is between positions 28 and 29, i.e., at the dash in the sequence VDT-CP.

TABLE 82B
NOV82 Polypeptide Sequence

| | |
|---|---|
| MVSVCRPWPAVAIALLALLVCLGALVDTCPIKPEAPGEDESLEELSHYYASLCHYLNVVT | (SEQ ID NO:272) |
| RQLISERNLPDTIVSKEVFFTSTKERPVRTQREGCHLQAKERSL | |

A BLAST analysis of NOV82 was run against the proprietary PatP GENESEQ Protein Patent database. It was found, for example, that the amino acid sequence of NOV82 had high homology to other proteins as shown in Table 82C.

TABLE 82C
BLASTX results from PatP database for NOV82

| | | Smallest |
|---|---|---|
| | | Sum |
| | High | Probability |
| Sequences producing High-scoring Segment Pairs: | Score | P(N) |
| patp: AAE09439 Human sbghPYYa protein - | 329 | 1.7e−29 |
| Homo sapiens | | |
| patp: AAB08020 Amino acid sequence of a | 301 | 1.6e−26 |
| human peptide yY | | |
| patp: AAG75364 Human colon cancer antigen | 293 | 1.1e−25 |
| protein | | |
| patp: AAY14602 Amino acid sequence of the | 221 | 4.7e−18 |
| baboon PY | | |
| patp: AAY43334 Neuropeptide Y - Synthetic, 97 aa. | 188 | 1.5e−14 |

In a search of sequence databases, it was found, for example, that the nucleic acid sequence of this invention has 217 of 288 bases (75%) identical to a gb:GENBANK-ID:HUMPYYP3|acc:D13902.1 mRNA from Homo sapiens (Human mRNA for peptide YY). The full amino acid sequence of the protein of the invention was found to have 62 of 94 amino acid residues (65%) identical to, and 73 of 94 amino acid residues (77%) similar to, the 97 amino acid residue ptnr:SWISSNEW-ACC:P10082 protein from Homo sapiens (Human) (PEPTIDE YY PRECURSOR (PYY)). NOV82 also has homology to the other proteins shown in the BLASTP data in Table 82D.

TABLE 82D
NOV82 BLASTP results

| Gene Index/ | | Length | Identity | Positive | |
|---|---|---|---|---|---|
| Identifier | Protein/Organism | (aa) | (%) | (%) | Expect |
| gi\|1172796\|sp\|P10082\| | PEPTIDE YY PRECURSOR | 97 | 62/94 | 73/94 | 2e−23 |
| PYY_HUMAN | (PYY) (PEPTIDE | | (65) | (76) | |
| | TYROSINE TYROSINE) | | | | |
| gi\|131753\|sp\|P10631\| | PEPTIDE YY PRECURSOR | 97 | 61/94 | 72/94 | 8e−23 |
| PYY_RAT | (PYY) (PEPTIDE | | (64) | (75) | |
| | TYROSINE TYROSINE) | | | | |
| gi\|4758982\|ref\|NP_{—} | peptide YY | 90 | 60/91 | 71/91 | 4e−22 |
| 004151.1\| | [Homo sapiens] | | (65) | (77) | |
| (NM_004160) | | | | | |
| gi\|422871\|pir\|\|S34569 | peptide YY precursor | 90 | 60/91 | 71/91 | 4e−22 |
| | (clone L2) - human | | (65) | (77) | |
| | (fragment) | | | | |
| gi\|422870\|pir\|\|S34568 | peptide YY precursor | 90 | 59/91 | 70/91 | 2e−21 |
| | (clone L1) - human | | (64) | (76) | |
| | (fragment) | | | | |

This BLASTP data is displayed graphically in the ClustalW in Table 82E. A multiple sequence alignment is given, with the NOV82 protein being shown on line 1 in a ClustalW analysis comparing the protein of the invention with the related protein sequences shown in Table 82D.

[image]

[image]

Table 82F lists the domain description from DOMAIN analysis results against NOV82. This indicates that the NOV82 sequence has properties similar to those of other proteins known to contain this domain.

TABLE 82F
Domain Analysis of NOV82

gn1|Pfam|pfam00159, hormone3, Pancreatic hormone
peptide SEQ ID NO:875
CD-Length = 36 residues, 91.7% aligned
Score = 43.1 bits (100), Expect = 8e−06

| | | | |
|---|---|---|---|
| NOV82: | 30 | PIKPEAPGEDESLEELSHYYASLCHYLNVVTRQ | 62 |
| | | P KPE PG+D S E+L+ Y +L Y+N++TR | |
| Sbjct: | 2 | PSKPEYPGDDASPEDLAQYLRALRQYINLITRP | 34 |

Pancreatic hormone (PP) is a peptide synthesized in pancreatic islets of Langherhans, which acts as a regulator of pancreatic and gastrointestinal functions. The hormone is produced as a larger propeptide, which is enzymatically cleaved to yield the mature active peptide, which is 36 amino acids in length and has an amidated C-terminus. The hormone has a globular structure with residues 2-8 forming a left-handed poly-proline-II-like helix, residues 9-13 a beta turn, and 14-32 an alpha-helix, held close to the first helix by hydrophobic interactions. Unlike glucagon, another peptide hormone, the structure of pancreatic peptide is preserved in aqueous solution. Both N- and C-termini are required for activity: receptor binding and activation functions may reside in the N- and C-termini respectively.

PYY is secreted from endocrine cells in the lower small intestine, colon, and pancreas. It acts on the gastrointestinal tract as an inhibitor of gastric acid secretion, gastric emptying, digestive enzyme secretion by the pancreas, and gut motility (Leiter et al., 1987). A related peptide, pancreatic polypeptide is secreted only by cells within the endocrine and exocrine pancreas and specifically inhibits the secretion of enzymes and bicarbonate from the exocrine pancreas. A third member of this gene family is neuropeptide Y.

Each of these proteins are synthesized with a signal peptide sequence followed by a 36-amino acid active peptide and a carboxyterminal peptide. During maturation, the signal and carboxyterminal peptides are cleaved and a common carboxyterminal tyrosine in the mature peptide is amidated. Hort et al. (1995) cloned the human PYY gene by screening a genomic library with a PCR product produced from the rat locus. The gene contains 4 exons spanning about 1.2-kb of DNA. Exon 1 represents 5-prime untranslated sequence and is 75% identical to the comparable rat sequence.

PYY and PPY are about 10-kb apart and are mapped them by fluorescence in situ hybridization to 17q21. Based on a comparison of the 3 gene sequences,it has been concluded that NPY and PYY are the result of a gene duplication event, and that a subsequent tandem duplication produced the PPY gene. Pancreatic polypeptide, peptide tyrosine-tyrosine (PYY), and neuropeptide tyrosine (NPY), three members of a family of structurally related peptides, are mainly expressed in the endocrine pancreas, in endocrine cells of the gut, and in the brain, respectively. Synthetic human PYY prepared using a solid-phase synthetic technique was found to be structurally identical to the natural peptide.

NOV82 is predicted to be expressed in at least the following tissues: endothelium, esophageal carcinoma. This information was derived by determining the tissue sources of the sequences that were included in the invention including but not limited to SeqCalling sources, public EST sources, literature sources, and/or RACE sources. Further expression data for NOV82 is provided in Example 2.

The novel nucleic acids of the invention encoding a PEPTIDE YY-like proteins includes the nucleic acid whose sequence is provided in Table 82A, or a fragment thereof. The invention also includes a mutant or variant nucleic acid any of whose bases may be changed from the corresponding base shown in Table 82A while still encoding a protein that maintains its PEPTIDE YY-like activities and physiological functions, or a fragment of such a nucleic acid. The invention further includes nucleic acids whose sequences are complementary to the sequence of Table 82A, including nucleic acid fragments that are complementary to any of the nucleic acids just described.

The invention additionally includes nucleic acids or nucleic acid fragments, or complements thereto, whose structures include chemical modifications. Such modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 25% of the bases may be so changed.

The novel protein of the invention includes the PEPTIDE YY-like protein whose sequence is provided in Table 82B. The invention also includes a mutant or variant protein any of whose residues may be changed from the corresponding residue shown in Table 82B while still encoding a protein that maintains its PEPTIDE YY-like activities and physiological functions, or a functional fragment thereof. In the mutant or variant protein, up to about 35% of the amino acid residues may be so changed.

The NOV82 nucleic acids and proteins are useful in potential therapeutic applications implicated in various pathological disorders described further herein, for example, esophageal carcinoma, inflammatory bowel disease, diverticular disease, as well as other diseases, disorders and conditions. NOV82 nucleic acids encoding the peptide YY-like protein of the invention, or fragments thereof, may further be useful in diagnostic applications, wherein the presence or amount of the nucleic acid or the protein are to be assessed.

These materials are further useful in the generation of antibodies that bind immunospecifically to the substances of the invention for use in therapeutic or diagnostic methods. These antibodies may be generated according to methods known in the art, using prediction from hydrophobicity charts, as described in the “Anti-NOVX Antibodies” section below.

NOV83

The disclosed NOV83 (alternatively referred to herein as CG56890-01) includes the 1701 nucleotide sequence (SEQ ID NO:273) shown in Table 83A. A NOV83 ORF begins with a Kozak consensus ATG initiation codon at nucleotides 19-21 and ends with a stop codon at nucleotides 1669-1671. The disclosed NOV83 maps to human chromosome 17.

TABLE 83A
NOV83 Nucleotide Sequence

(SEQ ID NO:273)

TGCGCCCCGTGCTCAGCCATGGTGGACATGGGGGCCCTGCACAACCTGATCGCCAACACCCCCTACCTGC
AGGCCCGGAAGCCCTCGGACTGCGACAGCAAAGAGCTGCAGCGGCGCCGGCGTAGCCTGGCCCTGCCCGG
GCTGCAGGGCTGCGCGGAGCTCCGCCAGAAGCTGTCCCTGAACTTCCACAGCCTGTGTGAGCAGCAGCCC
ATCGGTCGCCGCCTCTTCCGTGACTTCCTAGCCACAGTGCCCACGTTCCGCAAGGCGGCAACCTTCCTAG
AGGACGTGCAGAACTGGGAGCTGGCCGAGGAGGGACCCACCAAAGACAGCGCGCTGCAGGGGCTGGTGGC
CACTTGTGCGAGTGCCCCTGCCCCGGGGAACCCGCAACCCTTCCTCAGCCAGGCCGTGGCCACCAAGTGC
CAACCAGCCACCACTGAGGAAGAGCGAGTGGCTGCAGTGACGCTGGCCAAGGCTGAGGCCATGGCTTTCT
TGCAAGAGCAGCCCTTTAAGGATTTCGTGACCAGCGCCTTCTACGACAAGTTTCTGCAGTGGAAACTCTT
CGAGATGCAACCAGTGTCAGACAAGTACTTCACTGAGTTCAGAGTGCTCGGGAAAGGTGGTTTTGGGGAG
GTAAAAAACACTGGGAAGATGTATGCCTGTAAGAAACTGGACAAGAAGCGGCTGAAGAAGAAAGGTGGCG
AGAAGATGGCTCTCTTGGAAAAGCAAATCTTGGAGAAGGTCAGCAGCCCTTTCATTGTCTCTCTGGCCTA
TGCCTTTGAGAGCAAGACCCATCTCTGCCTTGTCATGAGCCTGATGAATGGGGGAGACCTCAACTTCCAC
ATCTACAACGTGGGCACGCGTGGCCTGGACATGAGCCGGGTGATCTTTTACTCGGCCCAGATACCCTGTG
GGATGCTGCACCTCCATGAACTCGGCATCGTCTATCGGGACATGAAGCCTGAGAATCTGCTTCTGGATGA
CCTCGGCAACTCCAGGTTATCTGACCTGCGGCTGGCCGTGGAGATGAAGGGTGGCAAGCCCATCACCCAG
AGGCAGGCTGGAACCAATGGTTACATGGCTCCTGAGATCCTAATGGAAAAGGTAAGTTATTCCTATCCTG
TGGACTGGTTTGCCATGGGATGCAGCATTTATGAAATGGTTGCTGGACGAACACCATTCAAAGATTACAA
GCAAAAGGTCAGTAAAGAGGATCTGAAGCAAAGAACTCTGCAAGACGAGGTCAAATTCCAGCATGATAAC
TTCACACAGGAAGCAAAAGATATTTCCAGGCTCTTCTTGGCTAAGAAACCAGAGCAACGCTTAGGAAGCA
GGAGAGAAAAGTCTGATCATCCCAGGAAACATCATTTCTTTAAAACGATCAACTTTCCTCCCCTGGAAGC
TGGCCTAATTGAACCCCCATTTGTGCCAGACCCTTCAGTGGTTTATGCCAAAGACATCGCTGAAATTGAT
GATTTCTCTGAGCTTCGGGGGGTGGAATTTGATGACAAAGATAAGCAGTTCTTCAAAAACTTTGCGACAG
GTGCTGTTCCTATAGCATGGCAGGAAGAAATTATAGAAACGGGACTGTTTGAGGAACTGAATGACCCCAA
CAGACCTACGGGTTGTGAGGAGGGTAATTCATCCAAGTCTGGCGTGTGTTTGTTATTGTAAATTGCTCTC
TTTACCAGACAGGCAGCAGGA

A NOV83 polypeptide (SEQ ID NO:274) encoded by SEQ ID NO:273 is 550 amino acids in length and is presented using the one-letter amino acid code in Table 83B. The Psort profile for NOV83 predicts that this sequence is likely to be localized to the nucleus with a certainty of 0.9685. In alternative embodiments, a NOV83 polypeptide is located to microbodies with a certainty of 0.1317.

TABLE 83B
NOV83 Polypeptide Sequence

| | |
|---|---|
| MVDMGALDNLIANTAYLQARKPSDCDSKELQRRRRSLALPGLQGCAELRQKLSLNFHSLC | (SEQ ID NO:274) |
| EQQPIGRRLFRDFLATVPTFRKAATFLEDVQNWELAEEGPTKDSALQGLVATCASAPAPG | |
| NPQPFLSQAVATKCQAATTEEERVAAVTLAKAEAMAFLQEQPFKDFVTSAFYDKFLQWKL | |
| FEMQPVSDKYFTEFRVLGKGGFGEVKNTGKMYACKKLDKKRLKKKGGEKMALLEKEILEK | |
| VSSPFIVSLAYAFESKTHLCLVMSLMNGGDLKFHIYNVGTRGLDMSRVIFYSAQIACGML | |
| HLHELGIVYRDMKPENVLLDDLGNCRLSDLGLAVEMKGGKPTTQRQAGTNGYMAPEILME | |
| KVSYSYPVDWFAMGCSIYEMVAGRTPFKDYKEKVSKEDLKQRTLQDEVKFQHDNFTEEAK | |
| DICRLFLAKKPEQRLGSRREKSDDPRKHHFFKTINFPRLEAGLIEPPFVPDPSVVYAKDI | |
| AEIDDFSEVRGVEFDDKDKQFFKNFATGAVPIAWQEEIIETGLFEELNDPNRPTGCEEGN | |
| SSKSGVCLLL | |

A BLAST analysis of NOV83 was run against the proprietary PatP GENESEQ Protein Patent database. It was found, for example, that the amino acid sequence of NOV83 had high homology to other proteins as shown in Table 83C.

TABLE 83C
BLASTX results from PatP database for NOV83

| | | Smallest |
|---|---|---|
| | | Sum |
| | High | Probability |
| Sequences producing High-scoring Segment Pairs: | Score | P(N) |
| patp: AAU03502 Human protein kinase #2 - | 2812 | 1.3e−292 |
| Homo sapiens | | |
| patp: AAY57085 Human rhodopsin kinase amino | 1255 | 1.3e−127 |
| acid sequence | | |
| patp: AAY24423 GRK4 polymorphism GRK4-alpha | 1248 | 7.0e−127 |
| protein | | |
| patp: AAY24424 GRK4 polymorphism GRK4-beta | 1190 | 1.7e−124 |
| protein | | |
| patp: AAY24425 GRK4 polymorphism | 1215 | 2.2e−123 |
| GRK4-gamma protein | | |

In a search of sequence databases, it was found, for example, that the nucleic acid sequence of this invention has 1359 of 1700 bases (79%) identical to a gb:GENBANK-ID:AF063016|acc:AF063016.1 mRNA from Spermophilus tridecemlineatus (Spermophilus tridecemlineatus G protein-coupled receptor kinase GRK7 mRNA). The full amino acid sequence of the protein of the invention was found to have 463 of 549 amino acid residues (84%) identical to, and 502 of 549 amino acid residues (91%) similar to, the 548 amino acid residue ptnr:SPTREMBL-ACC:Q9Z2G7 protein from Spermophilus tridecemlineatus (Thirteen-lined ground squirrel) (G PROTEIN-COUPLED RECEPTOR KINASE GRK7). NOV83 also has homology to the other proteins shown in the BLASTP data in Table 83D.

TABLE 83D
NOV83 BLASTP results

| Gene Index/ | | Length | | Positive | |
|---|---|---|---|---|---|
| Identifier | Protein/Organism | Identity (aa) | (%) | (%) | Expect |
| gi\|17026318\|gb\| | G protein-coupled | 553 | 548/555 | 548/555 | 0.0 |
| AAL33880.1\| | receptor kinase 7 [Homo | | (98) | (98) | |
| AF282269_1 | sapiens] | | | | |
| (AF282269) | | | | | |
| gi\|17933490\|gb\| | retina G protein-coupled | 552 | 470/555 | 503/555 | 0.0 |
| AAL06241.1\| | receptor kinase 7 | | (84) | (89) | |
| (AY049726) | [Bos taurus] | | | | |
| gi\|17026320\|gb\| | G protein-coupled | 553 | 465/555 | 504/555 | 0.0 |
| AAL33881.1\| | receptor kinase 7 [Sus | | (83) | (90) | |
| AF282270_1 | scrofa] | | | | |
| (AF282270) | | | | | |
| gi\|4001826\|gb\| | G protein-coupled | 548 | 463/554 | 502/554 | 0.0 |
| AAC95001.1\| | receptor kinase GRK7 | | (83) | (90) | |
| (AF063016) | [Spermophilus | | | | |
| | tridecemlineatus] | | | | |
| gi\|17443851\|ref\| | similar to G protein- | 692 | 326/350 | 328/350 | e−172 |
| XP_067593.1\| | coupled receptor kinase | | (93) | (93) | |
| (XM_067593) | GRK7 (H. sapiens) | | | | |
| | [Homo sapiens] | | | | |

This BLASTP data is displayed graphically in the ClustalW in Table 83E. A multiple sequence alignment is given, with the NOV83 protein being shown on line 1 in a ClustalW analysis comparing the protein of the invention with the related protein sequences shown in Table 83D.

[image]

[image]

[image]

Table 83F lists the domain description from DOMAIN analysis results against NOV83. This indicates that the NOV83 sequence has properties similar to those of other proteins known to contain this domain.

TABLE 83F
Domain Analysis of NOV83

gn1|Smart|smart00220, S_TKc, Serine/Threonine protein kinases, catalytic
domain; Phosphotransferases. Serine or threonine-specific kinase subfamily.
SEQ ID NO:876
CD-Length = 256 residues. 100.0% aligned
Score = 208 bits (530), Expect = 6e−55

| | | | |
|---|---|---|---|
| NOV83: | 191 | FTEFRVLGKGGFGEVKNTGKMYACKKLDICKRLKKKGGEKMALLEKEILEKVSSPF | 245 |
| | | + VLGKG FG+V K TGK+ A K + K++LKKK E L E +IL+K+ P | |
| Sbjct: | 1 | YELLEVLGKGAFGKVYLARDKKTGKLVAIKVIKKEKLKKKKRE-RILREIKILKKLDHPN | 59 |
| NOV83: | 246 | IVSLAYAFESKTHLCLVMSLMNGGDLKFHIYNVGTRGLDMSRVIFYSAQIACGMLHLHEL | 305 |
| | | IV L FE L LVM GGDL + G L FY+ QI + +LH | |
| Sbjct: | 60 | IVKLYDVFEDDDKLYLVMEYCECGDLFDLLKKRG--RLSEDEARFYARQILSALEYLHSQ | 117 |
| NOV83: | 306 | GIVYRDMKPENVLLDDLGNCRLSDLGLAVEMKCCKPITQRQAGTNGYMAPEILMEKVSYS | 365 |
| | | GI++RD+KPEN+LLD G+ +L+D GLA ++ G + GT YMAPE+L+ K Y | |
| Sbjct: | 118 | GIIHRDLKPENILLDSDGHVKLADFGLAKQLDSGGTLLTTFVGTPEYMAPEVLLGK--GYG | 176 |
| NOV83: | 366 | YPVDWFANGCSIYEMVAGRTPFKDYKEKVSKEDLKQRTLQDEVKF--QHDNFTEEAKDIC | 423 |
| | | VD +++G +YE++ G+ F + L ++ + F + EAKD+ | |
| Sbjct: | 177 | KAVDIWSLGVILYELLTGKPPFPGDDQ---LLALFKKICKPPPPFPPPEWKISPEAKDLI | 233 |
| NOV83: | 424 | RLFLAKKPEQRLGSRREKSDDPRKHHFF | 451 |
| | | + L K PE+RL +++ +H+FF | |
| Sbjct: | 234 | KKLLVKDPEKRLT-----AEEALENPFF | 256 |

Serine/threonine protein kinases are an extensive family of enzymes that catalyzes the phosphorylation of serine or threonine residues on its target protein. Protein kinases share a conserved catalytic core common to both serine/threonine and tyrosine protein kinases. This domain contains residues, which are specific to the distinct types of protein kinases

The S6/H4 kinase purified from human placenta catalyzes phosphorylation of the S6 ribosomal protein, histone H4, and myelin basic protein. In vitro activation of the p60 S6/H4 kinase requires removal of an autoinhibitory domain by mild trypsin digestion and autophosphorylation of the catalytic domain (p40 S6/H4 kinase). The two autophosphorylation/autoactivation sites contain the sequences SSMVGTPY (site 1) and SVIDPVPAPVGDSHVDGAAK (site 2) (SEQ ID NO:1381). These sequences identify S6H4 kinase as the rac-activated PAK65 (Martin, G. A., Bollag, G., McCormick, F. and Abo, A. (1995) EMBO J. 14, 1971-1978). Site 1 phosphorylation is most rapid, but activation does not occur until site 2 is autophosphorylated. The site 1 phosphorylation occurs by an intramolecular mechanism whereas site 2 autophosphorylation occurs by an intermolecular mechanism. A model is proposed in which phosphorylation of sites 1 and 2 occurs sequentially. The model proposes that trypsin treatment of the inactive holoenzyme removes an inhibitory rac-binding domain which blocks MgATP access to the catalytic site. The pseudosubstrate domain at site 1 is autophosphorylated and subsequent bimolecular autophosphorylation at site 2 fully opens the catalytic site. Phosphorylation by a regulatory protein kinase may occur at site 2 in vivo.

Rapid regulation of G-protein-coupled receptors (GPCRs) involves agonist-promoted receptor phosphorylation by GPCR kinases (GRKs). This process is followed by arrestin binding and transient receptor internalisation. It has been shown that beta-adrenergic receptor kinase (beta ARK-1 or GRK2) follows a similar pattern of internalisation upon agonist activation of beta(2)-adrenergic receptors (beta(2)AR) and that beta ARK expression levels modulate receptor sequestration.

Such studies indicate a functional relationship between receptor phosphorylation and sequestration, showing that beta ARK not only translocates from the cytoplasm to the plasma membrane in response to receptor occupancy, but also shares endocytic mechanisms with the beta(2)AR. These results suggest a role for beta ARK in the sequestration process, or involvement of receptor internalisation in the intracellular trafficking of the kinase.

NOV83 is predicted to be expressed in at least the following tissues: retina, spleen. This information was derived by determining the tissue sources of the sequences that were included in the invention including but not limited to SeqCalling sources, public EST sources, literature sources, and/or RACE sources. Further expression data for NOV83 is provided in Example 2.

The NOV83 nucleic acids and proteins are useful in potential therapeutic applications implicated in various pathological disorders described further herein, for example, hemophilia, hypercoagulation, idiopathic thrombocytopenic purpura, immunodeficiencies, graft vesus host disease, Von Hippel-Lindau (VHL) syndrome, diabetes, tuberous sclerosis, as well as other diseases, disorders and conditions. NOV83 nucleic acids encoding the GPCR-like protein of the invention, or fragments thereof, may further be useful in diagnostic applications, wherein the presence or amount of the nucleic acid or the protein are to be assessed.

The novel nucleic acid of the invention encoding a G protein-coupled receptor kinase GRK7-like protein includes the nucleic acid whose sequence is provided in Table 83A, or a fragment thereof. The invention also includes a mutant or variant nucleic acid any of whose bases may be changed from the corresponding base shown in Table 83A while still encoding a protein that maintains its G protein-coupled receptor kinase GRK7-like activities and physiological functions, or a fragment of such a nucleic acid. The invention further includes nucleic acids whose sequences are complementary to the sequence of Table 83A, including nucleic acid fragments that are complementary to any of the nucleic acids just described.

The invention additionally includes nucleic acids or nucleic acid fragments, or complements thereto, whose structures include chemical modifications. Such modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 21% of the bases may be so changed.

The novel protein of the invention includes the G protein-coupled receptor kinase GRK7-like protein whose sequence is provided in Table 83B. The invention also includes a mutant or variant protein any of whose residues may be changed from the corresponding residue shown in Table 83B while still encoding a protein that maintains its G Protein-Coupled Receptor Kinase GRK7-like activities and physiological functions, or a functional fragment thereof. In the mutant or variant protein, up to about 16% of the amino acid residues may be so changed.

These materials are further useful in the generation of antibodies that bind immunospecifically to the substances of the invention for use in therapeutic or diagnostic methods. These antibodies may be generated according to methods known in the art, using prediction from hydrophobicity charts, as described in the “Anti-NOVX Antibodies” section below.

NOV84

The disclosed NOV84 (alternatively referred to herein as CG56912-01) includes the 2355 nucleotide sequence (SEQ ID NO:275) shown in Table 84A. A NOV84 ORF begins with a Kozak consensus ATG initiation codon at nucleotides 11-13 and ends with a stop codon at nucleotides 2336-2338. The disclosed NOV84 maps to human chromosome 17.

TABLE 84A
NOV84 Nucleotide Sequence

(SEQ ID NO:275)

AGGCTGGAGGATGGGGCAGCAGGTCCTAGCGGGGGAGGGCAGCGGAGCCTCTGGGCAGGTACGGCCTGAC
GCCCCGGGTCCTCCCGCCCCGCCAGGCCTGACGGAGGACGAGGACGTGCGCGCCATGCTGCGGGGCTCCC
GGCTCCGCAAGATCCGCTCGCGCACGTGGCACAAGGAGCGGCTGTACCGGCTGCAGGAGGACGGCCTGAG
CGTCTGGTTCCAGCGGCGCATCCCGCGTGCGCCATCGCAGCACATCGTCCTCCGCCCTGACCCGGCCCTC
CTCTCAGTCTTCGTGCAGCACATCGAGGCGGTCCGCGAGGGCCACCAGTCCGAGGGCCTGCGGCGCTTCG
GGGGTGCCTTCGCGCCAGCGCGCTCCCTCACCATCGCCTTCAAGGGCCGCCGCAAGAACCTGGACCTGGC
GGCGCCCACGGCTGAGGAAGCGCAGCGCTGGGTGCGCGGTCTGACCAAGCTCCGCGCGCGCCTGGACGCC
ATGAGCCAGCGCGAGCGGCTAGACCAATATTGGATCCACTCCTATCTGCACCGGGCTGACTCCAACCAGG
ACAGCAAGATGAGCTTCAAGGAGATCAAGAGCCTGCTGAGAATGGTCAACGTGGACATGAACGACATGTA
CGCCTACCTCCTCTTCAAGCAGGAGTGTGACCACTCCAACAACGACCGTCTAGAGGGGGCTGAGATCGAG
GAGTTCCTGCGGCCGCTGCTGAAGCGGCCGGAGCTGGAGGAGATCTTCCATCAGTACTCGCGCGAGGACC
GCGTGCTGAGTGCCCCTGAGCTGCTGGAGTTCCTGGAGGACCAGGGCGAGGAGGGCGCCACACTGGCCCG
CGCCCACCAGCTCATTCACACCTATGAGCTCAACGAGACACCCTCTCCTGCCACCCCTATGACACTGGAT
GGCTTCATGATGTACCTGTTGTCGCCGGAGGGGGCTGCCTTGGACAACACCCACACGTGTGTGTTCCAGG
ACATGAACCAGCCCCTTGCCCACTACTTCATCTCTTCCTCCCACAACACCTATCTGACTGACTCCCAGAT
CGQGGGGCCCAGCAGCACCGAGGCCTATGTTAGGGCCTTTGCCCAGGGATGCCGCTGCGTGGAGCTGGAC
TGCTGGGAGGGGCCAGCAGGGGAGCCCGTCATCTATCATGGCCATACCCTCACCTCCAAGATTCTCTTCC
GGGACGTGGTCCAAGCCGTGCGCGACCATGCCTTCACGCTGTCCCCTTACCCTGTCATCCTATCCCTGGA
CAACCACGACGGGCTGGAGCAGCAGGCTGCCATGGCCCGCCACCTCTGCACCATCCTGGGGGACATGCTG
GTGACACAGGCGCTGGACTCCCCAAATCCCGAGGAGCTGCCATCCCCAGAGCAGCTGAAGGGCCGGGTCC
TGGTGAAGGGAAACAAGCTGCCCGCTGCTCGGAGCGAGGATGGCCGGGCTCTGTCGGATCGGGAGGAGGA
GGAGGAGGATGACGAGGAGGAGGAAGGAAAGGTGGAGGCTGCAGCGCAGAGGCGGCTGGCCAAGCAGATC
TCCCCGGAGCTGTCGGCCCTGGCTGTGTACTGCCACGCCACCCGCCTGCGGACCCTGCACCCTGCCCCCA
ACGCCCCACAACCCTGCCAGGTCAGCTCCCTCAGCGAGCGCAAAGCCAAGAAACTCATTCGGGAGGCAGG
GAACAGCTTTGTCAGGCACAATGCCCGCCAGCTGACCCGCGTGTACCCGCTGGGGCTGCGGATGAACTCA
GCCAACTACAGTCCCCAGGAGATGTGGAACTCGGGCTGTCAGCTGGTGGCCTTGAACTTCCAGACGCCAG
GCTACGAGATGGACCTCAATGCCGGGCGCTTCCTAGTCAATGGGCAGTGTGGCTACGTCCTAAAACCTGC
CTGCCTGCGGCAACCTGACTCGACCTTTGACCCCGAGTACCCAGGACCTCCCAGAACCACTCTCAGCATC
CAGGTGCTGACTGCACAGCAGCTGCCCAAGCTGAATGCCGAGAAGCCACACTCCATTGTGGACCCCCTGG
TGCGCATTGAGATCCATGGGGTGCCCGCAGACTGTGCCCGGCAGGAGACTGACTACGTGCTCAACAATGG
CTTCAACCCCCGCTGGGGGCAGACCCTGCAGTTCCAGCTGCGGGCTCCGGAGCTGGCACTGGTCCGGTTT
GTGGTGGAAGATTATGACGCCACCTCCCCCAATGACTTTGTGGGCCAGTTTACACTGCCTCTTAGCAGCC
TAAAGCAAGGGTACCGCCACATACACCTGCTTTCCAAGGACCGGGCCTCACTGTCACCAGCCACGCTCTT
CATCCAAATCCGCATCCAGCGCTCCTGAGGGCCCACCTCACTCGC

A NOV84 polypeptide (SEQ ID NO:276) encoded by SEQ ID NO:275 is 775 amino acids in length and is presented using the one-letter amino acid code in Table 84B. The Psort profile for NOV84 predicts that this sequence is likely to be localized to the cytoplasm with a certainty of 0.4500. In alternative embodiments, a NOV84 polypeptide is located to microbodies with a certainty of 0.3000.

TABLE 84B
NOV84 Polypeptide Sequence

| | |
|---|---|
| MGQQVLAGEGRGASGQVRPDAPGPPAPPGLTEDEDVRANLRGSRLRKIRSRTWHKERLYR | (SEQ ID NO:276) |
| LQEDGLSVWFQRRIPRAPSQHIVLRPDPALLSVFVQHIEAVREGHQSEGLRRFGGAFAPA | |
| RCLTIAFKGRRKNLDLAAPTAEEAQRWVRGLTRLRARLDANSQRERLDQYWIHSYLHRAD | |
| SNQDSKMSFKEIKSLLRNVNVDMNDMYAYLLFKQECDHSNNDRLEGAEIEEFLRRLLKRP | |
| ELEEIFHQYSGEDRVLSAPELLEFLEDQGEEGATLARAQQLIQTYELNETPSPATPMTLD | |
| GFMMYLLSPEGAALDNTHTCVFQDMNQPLAHYFISSSHNTYLTDSQIGGPSSTEAYVRAF | |
| AQGCRCVELDCWEGPGGEPVIYHGHTLTSKILFRDVVQAVRDHAFTLSPYPVILSLDNHD | |
| GLEQQAANARHLCTILGDMLVTQALDSPNPEELPSPEQLKGRVLVKGKKLPAARSEDGRA | |
| LSDREEEEEDDEEEEEEVEAAAQRRLAKQISPELSALAVYCHATRLRTLHPAPNAPQPCQ | |
| VSSLSERKAKKLIREAGNSFVRHNARQLTRVYPLGLRMNSANYSPQEMWNSGCQLVALNF | |
| QTPGYEMDLNAGRFLVNGQCGYVLKPACLRQPDSTFDPEYPGPPRTTLSIQVLTAQQLPK | |
| LNAEKPHSIVDPLVRIEIHGVPADCARQETDYVLNNGFNPRWGQTLQFQLRAPELALVRF | |
| VVEDYDATSPNDFVGQFTLPLSSLKQGYRHIHLLSKDGASLSPATLFIQIRIQRS | |

A BLAST analysis of NOV84 was run against the proprietary PatP GENESEQ Protein Patent database. It was found, for example, that the amino acid sequence of NOV84 had high homology to other proteins as shown in Table 84C.

TABLE 84C
BLASTX results from PatP database for NOV84

| | | Smallest |
|---|---|---|
| | | Sum |
| | High | Probability |
| Sequences producing High-scoring Segment Pairs: | Score | P(N) |
| patp: AAG63220 Amino acid sequence of a | 3735 | 0.0 |
| human protein | | |
| patp: AAB47516 Human phospholipase C, 16835 - | 3734 | 0.0 |
| Homo sapiens | | |
| patp: AAY81394 Rat phospholipase C-delta-1 - | 1882 | 4.6e−194 |
| Rattus sp. | | |
| patp: AAE10440 Novel human phospholipase | 1783 | 1.4e−183 |
| protein #7 | | |
| patp: AAE11925 Human CG121 (or C592) lipase | 1182 | 6.9e−120 |
| protein #1 | | |

In a search of sequence databases, it was found, for example, that the nucleic acid sequence of this invention has 954 of 1415 bases (67%) identical to a gb:GENBANK-ID:OCPLCMR|acc:Z49747.1 mRNA from Oryctolagus cuniculus (O.cuniculus mRNA for phospholipase C). The full amino acid sequence of the protein of the invention was found to have 381 of 745 amino acid residues (51%) identical to, and 524 of 745 amino acid residues (70%) similar to, the 745 amino acid residue ptnr:SPTREMBL-ACC:Q60450 protein from Cricetulus griseus (Chinese hamster) (PHOSPHOLIPASE C-DELTA1). NOV84 also has homology to the other proteins shown in the BLASTP data in Table 84D.

TABLE 84D
NOV84 BLASTP results

| Gene Index/ | | | Identity | Positive | |
|---|---|---|---|---|---|
| Identifier | Protein/Organism | Length (aa) | (%) | (%) | Expect |
| gi\|14715017\|gb\| | Similar to phospholipase | 613 | 604/615 | 606/615 | 0.0 |
| AAH10668.1\| | C, delta [Homo sapiens] | | (98) | (98) | |
| AAH10668 | | | | | |
| (BC010668) | | | | | |
| gi\|17481372\|ref\| | hypothetical protein | 581 | 472/500 | 475/500 | 0.0 |
| XP_053638.2\| | XP_053638 | | (94) | (94) | |
| (XM_053638) | [Homo sapiens] | | | | |
| gi\|2137061\|pir\|\| | 1-phosphatidylinositol- | 745 | 378/758 | 522/758 | 0.0 |
| PC4183 | 4,5-bisphosphate | | (49) | (67) | |
| | phosphodiesterase (EC | | | | |
| | 3.1.4.11) delta-1 - | | | | |
| | Chinese hamster | | | | |
| | (fragment) | | | | |
| gi\|8393981\|ref\| | phospholipase C-delta1 | 756 | 368/757 | 501/757 | 0.0 |
| NP_058731.1\| | [Rattus norvegicus] | | (48) | (65) | |
| (NM_017035) | | | | | |
| gi\|9790167\|ref\| | phospholipase C, delta; | 756 | 374/757 | 503/757 | 0.0 |
| NP_062650.1\| | PLC-delta 1; | | (49) | (66) | |
| (NM_019676) | phospholipase C delta-1 | | | | |
| | [Mus musculus] | | | | |

This BLASTP data is displayed graphically in the ClustalW in Table 84E. A multiple sequence alignment is given, with the NOV84 protein being shown on line 1 in a ClustalW analysis comparing the protein of the invention with the related protein sequences shown in Table 84D.

[image]

[image]

[image]

[image]

Table 84F lists the domain description from DOMAIN analysis results against NOV84. This indicates that the NOV84 sequence has properties similar to those of other proteins known to contain this domain.

TABLE 84F
Domain Analysis of NOV84

gn1|Smart|smart00148, PLCXc, Phospholipase C, catalytic domain (part); domain
X; Phosphoinositide-specific phospholipases C. These enzymes contain 2
regions (X and Y) which together form a TIM barrel-like structure containing
the active site residues. Phospholipase C enzymes (P1-PLC) act as signal
transducers that generate two second messengers, inositol-1,4,5-trisphosphate
and diacylglycerol. The bacterial enzyme appears to be a homologue of the
mammalian PLCs. SEQ ID NO:877
CD-Length = 145 residues, 93.1% aligned
Score = 186 bits (471), Expect = 6e−48

| | | | |
|---|---|---|---|
| NOV84: | 323 | QDMNQPLAHYFISSSHNTYLTDSQIGGPSSTEAYVRAFAQGCRCVELDCWEGPGGEPVIY | 382 |
| | | QDM++PL+HYFI+SSHNTYLT Q+ G SS E Y++A GCRCVELDCW+GP GEPVIY | |
| Sbjct: | 1 | QDMSKPLSHYFINSSHNTYLTGKQLWCESSVEGYIQALKHGCRCVELDCWDGPDGEPVIY | 60 |
| NOV84: | 383 | HGNTLTSKILFRDVVQAVRDHAFTLSPYPVILSLDNHDGLEQQA HLCTILGDMLVT | 442 |
| | | HGNT T I +V++A++ AF SPYPVILSL+NH +QQA MA+ I GD+L T | |
| Sbjct: | 61 | HGHTFTLPIKLSEVLEAIKKFAFVTSPYPVILSLENHCSPDQQAKMAQMFKEIFGDLLYT | 120 |
| NOV84: | 443 | QALDSPNPEELPSPEQ | 458 |
| | | S + E LPSPEQ | |
| Sbjct: | 121 | PPTTS-SLEYLPSPEQ | 135 |

Phosphoinositide-specific phospholipase C acts as a signal transducer that generates two messengers, diacylglycerol and inositol 1,4,5-trisphosphate, by hydrolyzing inositol phospholipids. Molecules belonging to the PLC family are divided into subfamilies, PLC-beta, PLC-gamma, and PLC-delta, whose amino acid sequences are highly conserved in two distinct regions designated X and Y. PLC-delta-1 is distinguished from PLC-gamma by lack of the SH2 and SH3 domains that are essential for activation of PLC-gamma by tyrosine protein kinases, and from PLC-beta by lack of the C-terminal region of PLC-beta that is responsible for binding and activation by G proteins.

Cheng et al. (1995) cloned cDNA for human PLC-delta-1 and localized the gene to chromosome 3 by means of a human/rodent somatic cell panel (Lyu et al., 1996). In the course of a large-scale sequencing analysis of genomic DNA in the vicinity of the homozygous deletion on chromosome 3p found in a lung cancer cell line, Ishikawa et al. (1997) found that the gene encoding phospholipase C, delta-1 (PLCD1) is located just distal to the region removed by the deletion. They found that the gene consists of 15 exons and spans about 22 kb. By fluorescence in situ hybridization, they localized the PLCD1 gene to 3p22-p21.3. Shimohama et al. (1998) examined the entire sequences corresponding to protein-coding exons 2-15 of the hamster PLC-delta-1 gene in genomic DNA derived from the leukocytes of 13 unrelated patients with early-onset sporadic Alzheimer disease. In 1 of these patients whose clinical features and course did not differ from those of the other 12 cases, they found a change of codon CGC (arg) to CAC (his), located in the pleckstrin homology domain of the PLCD1 gene. They stated that this was the first mutation found in the human PLC genes.

Site-directed mutagenesis of the glutathione-S-transferase (GST/PLCD1) fusion protein changing arg105 to his resulted in a 4-fold decrease in the affinity of specific binding and a reduction in hydrolyzing activity to about 40% of that of the wildtype enzyme. This remarkable loss of function could be interpreted in terms of a conformational change in the pleckstrin homology domain. Shimohama et al. (1998) found that the arg105-to-his mutation was present in heterozygous state in the patient with AD. The mutation was not found in DNA extracted from leukocytes of 23 unrelated patients with familial AD, 23 unrelated patients with early-onset sporadic AD, 46 unrelated patients with late-onset sporadic AD, and 456 nondemented control subjects. Thus the change did not appear to be a common polymorphism.

However, determination of the possible pathologic role required transgenic studies of the mutant gene to determine the role of the enzyme and the mutation and a search for other mutations in the pleckstrin homology domain of PLC genes in human subjects with genetic disorders. In vitro single point mutagenesis, inositol phospholipid hydrolysis, and substrate protection experiments were used to identify catalytic residues of human phosphatidylinositide-specific phospholipase C delta 1 (PLC delta 1) isolated from a human aorta cDNA library. Invariant amino acid residues containing a functional side chain in the highly conserved X region were changed by in vitro mutagenesis. Most of the mutant enzymes were still able to hydrolyze inositol phospholipid with activity ranging from 10 to 100% of levels in the wild type enzyme. Exceptions were mutants with the conversion of Arg338 to Leu (R338L), Glu341 to Gly (E341G), or His356 to Leu (H356L), which made the enzyme severely defective in hydrolyzing inositol phospholipid. Phospholipid vesicle binding experiments showed that these three cleavage-defective mutant forms of PLC delta 1 could specifically bind to phosphatidylinositol 4,5-bisphosphate (PIP2) with an affinity similar to that of wild type enzyme. Western blotting analysis of trypsin-treated enzyme-PIP2 complexes revealed that a 67-kDa major protein fragment survived trypsin digestion if the wild type enzyme, E341G, or H356L mutant PLC delta 1 was preincubated with 7.5 microM PIP2, whereas if it was preincubated with 80 microM PIP2, the size of major protein surviving was comparable to that of intact enzyme. However, mutant enzyme R338L was not protected from trypsin degradation by PIP2 binding. These observations suggest that PLC delta 1 can recognize PIP2 through a high affinity and a low affinity binding site and that residues Glu341 and His356 are not involved in either high affinity or low affinity PIP2 binding but rather are essential for the Ca(2+)-dependent cleavage activity of PLC.

NOV84 is predicted to be expressed in at least the following tissues: aorta, brain, colon, foreskin, heart, muscle, placenta, stomach, uterus, whole embryo, brain, colon, eye, head and neck, lung, muscle, ovary, pancreas, placenta, skin, stomach, uterus. This information was derived by determining the tissue sources of the sequences that were included in the invention including but not limited to SeqCalling sources, public EST sources, literature sources, and/or RACE sources. Further expression data for NOV84 is provided in Example 2.

The NOV84 nucleic acids and proteins are useful in potential therapeutic applications implicated in various pathological disorders described further herein, for example, cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, transplantation as well as other diseases, disorders and conditions. NOV84 nucleic acids encoding the phospholipase-like protein of the invention, or fragments thereof, may further be useful in diagnostic applications, wherein the presence or amount of the nucleic acid or the protein are to be assessed.

The novel nucleic acid of the invention encoding a Phospholipase C delta 1-like protein includes the nucleic acid whose sequence is provided in Table 84A, or a fragment thereof. The invention also includes a mutant or variant nucleic acid any of whose bases may be changed from the corresponding base shown in Table 84A while still encoding a protein that maintains its Phospholipase C delta 1-like activities and physiological functions, or a fragment of such a nucleic acid. The invention further includes nucleic acids whose sequences are complementary to the sequence of Table 84A, including nucleic acid fragments that are complementary to any of the nucleic acids just described.

The invention additionally includes nucleic acids or nucleic acid fragments, or complements thereto, whose structures include chemical modifications. Such modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 33% of the bases may be so changed.

The novel protein of the invention includes the Phospholipase C delta 1-like protein whose sequence is provided in Table 84B. The invention also includes a mutant or variant protein any of whose residues may be changed from the corresponding residue shown in Table 84 while still encoding a protein that maintains its Phospholipase C delta 1-like activities and physiological functions, or a functional fragment thereof. In the mutant or variant protein, up to about 49% of the amino acid residues may be so changed.

These materials are further useful in the generation of antibodies that bind immunospecifically to the substances of the invention for use in therapeutic or diagnostic methods. These antibodies may be generated according to methods known in the art, using prediction from hydrophobicity charts, as described in the “Anti-NOVX Antibodies” section below.

NOV85

The disclosed NOV85 (alternatively referred to herein as CG56955-01) includes the 4091 nucleotide sequence (SEQ ID NO:277) shown in Table 85A. A NOV85 ORF begins with a Kozak consensus ATG initiation codon at nucleotides 13-15 and ends with a stop codon at nucleotides 4078-4080.

TABLE 85A
NOV85 Nucleotide Sequence

(SEQ ID NO:277)

ACTACTTGTGGAATGTCACTGCCTCGGGGTATTTCACAGACAGGTCACCTCTTGTGAAAAGTCCGAAGTA
ATTCTCTCAAAGCTCCTTCCACGCATGTCACAAAACCATCATTTAGCCAGAAATCATTTGTTTCTATGAG
AGACCAAAGACCAGTAAATCACTTGCATCAGAACAGTCTGTTGAATCAGCAGACATGGGTAAGGACTGAC
AGTGCCCCCGATCACCAAGTGGAGACTGGGAAATCCCCCTCTTTATCTGGAGCCTCTGCCAAGCCTGCCC
CTCAGTCGAGTGAAAACGCTGGTACTTCAGATTTAGAACTACCTGTCAGTCAAAGGAATCAAGATTTAAG
TTTACAAGAGGCTGAAACTGAGCAATCAGATACTTTAGATAATAAAGAAGCTGTCATCCTAAGGGAAAAA
CCTCCATCTGGACGCCAGACACCGCAGCCTTTAAGGCATCAGTCTTACATCTTGGCAGTAAATGACCAGG
AGACCGGGTCAGACACTACCTGCTGGCTGCCCAATGATGCACGTCGAGAGGTCCACATAAAAAGAATGGA
GGAAAGAAAAGCCTCGAGTACCAGTCCGCCTGGCGATTCTTTGGCTTCCATCCCATTTATAGATGAACCA
ACTAGCCCTAGCATTGATCATGATATTGCACATATCCCTGCCTCTGCTGTTATATCAGCCTCTACCTCTC
AGGTCCCCTCCATAGCAACAGTTCCTCCTTGCCTCACAACTTCAGCTCCATTAATTCGCCGTCAGCTCTC
ACATGACCACGAATCTGTTGGCCCTCCTAGCCTGGATGCTCAGCCCAACTCAAAGACAGAAAGATCAAAA
TCATATGATGAGGGTCTGGATGATTACAGAGAAGATGCAAAATTGTCCTTTAAGCACGTATCTAGTCTGA
ACGGAATCAAGATCGCAGACAGCCAAAAGTCATCAGAAGACTCTGGGTCCAGAAAAGATTCTTCCTCAGA
GGTCTTCAGTGATGCTGCCAAGGAAGGGTGGCTTCATTTCCGACCCCTTGTCACCGATAAGGGCAAGCGA
GTTGGTGGAAGTATTCGGCCATGGAAACAGATGTATGTTGTCCTTCGGGGTCATTCACTTTACCTGTACA
AAGATAAAAGAGAGCAGACGACTCCGTCTGAGGAAGAGCAGCCCATCAGTGTTAATGCTTGCTTGATAGA
CATCTCTTACAGTGAGACCAAGAGGAAAAATGTGTTTCGACTCACCACGTCCGACTGTGAATGCCTGTTT
CAGGCTGAAGACAGAGATGATATGCTAGCTTGGATCAAGACGATCCAGGAGAGCAGCAACCTAAACGAAG
AGGACACTGGAGTCACTAACAGGGATCTAATTAGTCGAAGAATAAAAGAATACAACAATCTGATGAGCAA
AGCAGAACAGTTGCCAAAAACACCTCGCCAGAGTCTCAGCATCAGGCAAACTTTGCTTGGTGCTAAATCA
GAGCCAAAGACTCAAAGCCCACACTCTCCGAAGGAACAGTCGGAAAGGAACTTCTCAGTAAGAAATGATA
CCAGTCCCCCAAAAGACAAAGGCACATGGAGAAAAGGCATTCCAAGTATCATGAGAAAGACATTTGAGAA
AAAGCCAACTGCTACAGGAACTTTCGGCGTCCGACTAGATGACTGCCCACCAGCTCATACTAATCGGTAT
ATTCCATTAATAGTTGACATATGTTGCAAATTAGTTGAAGAAAGAGGTCTTGAATATACAGGTATTTATA
GAGTTCCTGGAAATAATGCAGCCATCTCAAGTATGCAAGAAGAACTCAACAAGGGAATGGCTGATATTGA
TATACAAGATGATAAATGGCGAGATTTGAATGTGATAAGCAGTTTACTAAAATCCTTCTTCAGAAAACTC
CCTGAGCCTCTCTTCACAAATGATAAATATGCTGATTTTATTGAAGCCAATCGTAAAGAAGATCCTCTAG
ATCGTCTGAAAACATTAAAAAGACTAATTCACGATTTGCCTGAACATCATTATGAAACACTTAAGTTCCT
TTCAGCTCATCTGAAGACAGTGGCAGAAAATTCAGAGAAAAAAAATAAGATGGAACCAAGAAACCTAGCA
ATAGTGTTTGGTCCCACCCTTGTTCGAACATCAGAAGACAACATGACCCACATGGTCACCCACATGCCTG
ACCAGTACAAGATTGTAGAAACGCTCATCCAGCACCATGACTGGTTTTTCACAGAAGAAGGTGCTGAAGA
GCCTCTTACAACAGTGCAGGAGGAAAGCACAGTAGACTCCCAGCCAQTGCCAAACATAGATCATTTACTC
ACCAACATTGGAAGGACAGGAGTCTCCCCAGGAGATGTATCAGATTCAGCTACTAGTGACTCAACAAAAT
CTAAGGGTTCTTGGGCATCTGGAAAGGATCAGTATAGCAGGGAACTGCTTGTGTCCTCCATCTTTGCAGC
TGCTAGTCGCAAGAGGAAGAAGCCGAAAGAAAAAGCACAGCCTAGCAGCTCAGAAGATGAACTGGACAAT
GTATTTTTTAAGAAAGAAAATGTGGAACAGTGTCACAATGATACTAAAGAGGAGTCCAAAAAAGAAAGTG
AGACACTGGGCAGAAAACAGAAGATCATCATTGCCAAAGAAAACAGCACTAGGAAAGACCCCAGCACGAC
AAAAGATGAAAAGATATCACTAGGAAAAGAGAGCACGCCTTCTGAAGAACCCTCACCACCACACAACTCA
AAACACAACAAGTCACCAACTCTCACCTGTCGCTTTGCCATCCTGAAAGAGAGCCCCAGGTCACTTCTGG
CACAGAAGTCCTCCCACCTTGAAGACACAGGCTCTGACTCTGGCACTTTGCTCAGCACGTCTTCCCAGGC
CTCCCTGGCAAGGTTTTCCATGAAGAAATCAACCAGTCCAGAAACGAAACATAGCGAGTTTTTGGCCAAC
GTCAGCACCATCACCTCAGATTATTCCACCACATCGTCTGCTACATACTTGACTAGCCTGGACTCCAGTC
GACTGACCCCTGAGGTGCAATCCGTGGCAGAGAGCAAGGGGGACGAGGCAGATGACGAGAGAAGCGAACT
CATCAGTGAAGGGCGGCCTGTGGAAACCGACAGCGAGAGCGAGTTTCCCGTGTTCCCCACAGCCTTGACT
TCAGAGAGGCTTTTCCGAGGAAACTGCAAGAAGTGACTAAGAGCAGCCGGAGAATTCTGAAGGAAAAGTG
ATTAAGTTGCACCGAGGGAAGTTTAACATCAAGTTTAGATAGCCGGAGACAGCTCTTCAGTTCCCATAAA
ACTCATCGAATGTGATACTCTTTCCAGGAAAAAATCAGCTACATTCAAGTCAGATAGTGGAAGTCTAGGA
GATGCCAAGAATGAGAAAGAAGCACCTTCGTTAACTAAAGTGTTTGATGTTATGAAAAAAGGAAAGTCAA
CTGGGAGTTTACTGACACCCACCAGAGGCGAATCCGAAAAACAGGAACCCACATGGAAAACGAAAATAGC
AGATCGGTTAAAACTGAGACCCAGAGCCCCTGCGGATGACATGTTTGGAGTAGGGAATCACAAAGTGAAT
GCCGAGACTGCTAAAAGGAAAAGCATCCGGCGCAGACATACACTAGGAGGGCACAGAGATGCTACCGAAA
TCAGCGTTTTGAATTTTTGGAAAGTGCATGAGCAGAGCGGGGAGAGAGAATCTGAACTTTCAGCTGTAAA
CCGGTTAAAACCAAAATGCTCAGCCCAGGACCTTTCCATCTCAQACTGGCTGGCCAGGGAACGCCTACGC
ACCAGTACCTCTCACCTTAGCAGAGGAGAAATCGGAGATCCCCAGACAGAGAACCCAAGCACACGAGAAA
TAGCCACGACCGACACACCTTTGTCTCTTCATTGCAACACAGGCAGTTCTTCCAGCACCTTGGCTTCAAC
AAACAGGCCCCTTCTTTCCATACCACCACAGTCACCTGACCAAATAAACGGAGAAAGCTTCCAGAACGTG
AGCAAAAATGCTAGTTCTGCAGCGAATGCCCAACCTCATAAACTGTCTGAAACCCCAGGCACTAAAGCAG
AGTTTCATCCCTGTCTTTAAACTGGGGGTAT

A NOV85 polypeptide (SEQ ID NO:278) encoded by SEQ ID NO:277 is 1355 amino acids in length and is presented using the one-letter amino acid code in Table 85B. The Psort profile for NOV85 predicts that this sequence is likely to be localized to the nucleus with a certainty of 0.7000. In alternative embodiments, a NOV85 polypeptide is located to the mitochondrial matrix space with a certainty of 0.3600, or to microbodies with a certainty of 0.3000.

TABLE 85B
NOV85 Polypeptide Sequence

| | |
|---|---|
| MSLPRGISQDRSPLVKVRSNSLKAPSTHVTKPSFSQKSFVSMAAQRPAAHLHQNSLLNQQ | (SEQ ID NO:278) |
| TWVRTDSAPDQQVETGKSPSLSGASAKPAPQSSENAGTSDLELPVSQAAQDLSLQEAATE | |
| QSDTLDNKEAVILREKPPSGRQTPQPLRHQSYILAVNDQETGSDTTCWLPNDARREVHIK | |
| RNEERKASSTSPPGDSLASIPFIDEPTSPSIDHDIAHIPASAVISASTSQVPSIATVPPC | |
| LTTSAPLIRRQLSHDHESVGPPSLDAQPNSKTERSKSYDEGLDDYREDAKLSFKHVSSLK | |
| GIKIADSQKSSEDSGSRKDSSSEVFSDAAKEGWLHFRPLVTDKGKRVGGSTRPWKQMYVV | |
| LRGHSLYLYKDKREQTTPSEEEQPISVNACLIDISYSETKRKNVFRLTTSDCECLFQAED | |
| RDDMLAWIKTIQESSNLNEEDTGVTNRDLISRRIKEYNNLMSKAEQLPKTPRQSLSIRQT | |
| LLGAKSEPKTQSPHSPKEESERKLLSKDDTSPPKDKGTWRKGIPSIMRKTFEKKPTATGT | |
| FGVRLDDCPPAHTNRYIPLIVDICCKLVEERGLEYTGIYRVPGNNAAISSMQEELNKGAA | |
| DIDIQDDKWRDLNVISSLLKSFFRKLPEPLFTNDKYADFIEANRKEDPLDRLKTLKRLIH | |
| DLPEHHYETLKFLSAHLKTVAENSEKKNKMEPRNLAIVFGPTLVRTSEDNMTHMVTHMPD | |
| QYKIVETLIQHHDWFFTEEGAEEPLTTVQEESTVDSQPVPNIDHLLTNIGRTGVSPCDVS | |
| DSATSDSTKSKGSWGSGKDQYSRELLVSSIFAAASRKRKKPKEKAQPSSSEDELDNVFFK | |
| KENVEQCHNDTKEESKKESETLCRKQKIIIAKENSTRKDPSTTKDEKISLGKESTPSEEP | |
| SPPHNSKHNKSPTLSCRFAILKESPRSLLAQKSSHLEETGSDSGTLLSTSSQASLAAFSM | |
| KKSTSPETKHSEFLANVSTITSDYSTTSSATYLTSLDSSRLSPEVQSVAASKCDEAADER | |
| SELISEGRPVETDSESEFPVFPTALTSERLFRGKLQEVTKSSRAASEGSELSCTEGSLTS | |
| SLDSRRQLFSSHKLIECDTLSRKKSARFKSDSGSLGDAKNEKEAPSLTKVFDAAKKGKST | |
| GSLLTPTRGESEKQEPTWKTKIADRLKLRPAAPADDMFGVGAAKAAAETAKRKSIRREET | |
| LGGHRDATEISVLNFWKVHEQSGERESELSAVNRLKPKCSAQDLSISDWLARERLRTSTS | |
| DLSRGEIGDPQTENPSTREIATTDTPLSLHCNTGSSSSTLASTNRPLLSIPPQSPDQING | |
| ESFQNVSKNASSAANAQPHKLSETPGTKAEFHPCL | |

A BLAST analysis of NOV85 was run against the proprietary PatP GENESEQ Protein Patent database. It was found, for example, that the amino acid sequence of NOV85 had high homology to other proteins as shown in Table 85C.

TABLE 85C
BLASTX results from PatP database for NOV85

| | | Smallest |
|---|---|---|
| | | Sum |
| | High | Probability |
| Sequences producing High-scoring Segment Pairs: | Score | P(N) |
| patp: AAB97911 Human G-protein activating | 6012 | 0.0 |
| protein | | |
| patp: AAB41660 Human ORFX ORF1424 | 4368 | 0.0 |
| polypeptide sequence | | |
| patp: AAM93705 Human polypeptide | 852 | 3.9e−84 |
| patp: AAU17101 Novel signal transduction | 618 | 6.3e−59 |
| pathway protein | | |
| patp: AAB64387 Amino acid sequence of | 426 | 2.1e−38 |
| human intracellular | | |

In a search of sequence databases, it was found, for example, that the nucleic acid sequence of this invention has 3871 of 3875 bases (99%) identical to a gb:GENBANK-ID:AB037845|acc:AB037845.1 mRNA from Homo sapiens (mRNA for KIAA1424 protein). The full amino acid sequence of the protein of the invention was found to have 1285 of 1287 amino acid residues (99%) identical to, and 1286 of 1287 amino acid residues (99%) similar to, the 1286 amino acid residue ptnr:SPTREMBL-ACC:Q9P2C3 protein from Homo sapiens (Human) (KIAA1424 PROTEIN). NOV85 also has homology to the other proteins shown in the BLASTP data in Table 85D.

TABLE 85D
NOV85 BLASTP results

| Gene Index/ | | Length | Identity | Positive | |
|---|---|---|---|---|---|
| Identifier | Protein/Organism | (aa) | (%) | (%) | Expect |
| gi\|7243229\|dbj\| | KIAA1424 protein | 1286 | 1285/1287 | 1286/1287 | 0.0 |
| BAA92662.1\| | [Homo sapiens] | | (99) | (99) | |
| (AB037845) | | | | | |
| gi\|14736381\|ref\| | hypothetical protein | 1173 | 1172/1174 | 1173/1174 | 0.0 |
| XP_038564.1\| | XP_038564 | | (99) | (99) | |
| (XM_038564) | [Homo sapiens] | | | | |
| gi\|7959263\|dbj\| | KIAA1501 protein | 735 | 331/685 | 433/685 | e−148 |
| BAA96025.1\| | [Homo sapiens] | | (48) | (62) | |
| (AB040934) | | | | | |
| gi\|14748447\|ref\| | hypothetical protein | 304 | 277/284 | 279/284 | e−135 |
| XP_041405.1\| | XP_041405 | | (97) | (97) | |
| (XM_041405) | [Homo sapiens] | | | | |
| gi\|12856667\|dbj\| | KIAA1424 PROTEIN | 385 | 301/384 | 328/384 | e−127 |
| BAB30742.1\| | (FRAGMENT)˜putative [Mus | | (78) | (83) | |
| (AK017433) | musculus] | | | | |

This BLASTP data is displayed graphically in the ClustalW in Table 85E. A multiple sequence alignment is given, with the NOV85 protein being shown on line 1 in a ClustalW analysis comparing the protein of the invention with the related protein sequences shown in Table 85D.

[image]

[image]

[image]

[image]

[image]

Table 85F lists the domain description from DOMAIN analysis results against NOV85. This indicates that the NOV85 sequence has properties similar to those of other proteins known to contain this domain.

TABLE 85F
Domain Analysis of NOV85

gn1|Smart|smart00324, RhoGAP, GTPase-activator protein for Rho-like GTPases;
GTPase activator proteins towards Rho/Rac/Cdc42-Iike small GTPases.
SEQ ID NO:878
CD-Length = 175 residues, 100.0% aligned
Score = 169 bits (428), Expect = 9e−43

| | | | |
|---|---|---|---|
| NOV85: | 555 | RYIPLIVDICCKLVEERGLEYTGIYRVPGNNAAISSMQEEWKGAADIDIQDDKWAALAA | 614 |
| | | + IP+IV+ C + +E+RGL+ GIYR G+ + + ++E +G D D++ | 614 |
| Sbjct: | 1 | KPIPIIVEKCIEYLEKRGLOTEGIYRKSGSASRVKELREAFDSGPDPD--LDLSEYDVHD | 58 |
| NOV85: | 615 | ISSLLKSFFRKLPEPLFTNDKYADFIEANRKEDPLDRLKTLKRLIHDLPEHHYETLKFLS | 674 |
| | | ++ LLK F R+LPEPL T + Y +FIEA + ED +RL+ L+ L+ LP + TL++L | |
| Sbjct: | 59 | VAGLLKLFLRELPEPLITFELYEEFIEAAKLEDEEERLAAIAARELLSLLPPAAAATLRYLL 118 | |
| NOV85: | 675 | AHLKTVAENSEKKNKMEPRNLAIVFGPTLVRTSEDNMTHMVTHMPDQYKIVETLIQHHD | 733 |
| | | AHL VAE+SE +NKM RNLAIVFGPTL+R + + + A K+VE LI++ D | |
| Sbjct: | 119 | AHLNRVAEHSEAAENKMTARNLAIVFGPTLLRPPDGESASLKDIRHQNKAAEFLIEND | 175 |

Rho GTPases control a variety of cellular processes. There are three subtypes of Rho GTPases in the Ras superfamily of small G proteins: RHO, RAC, and CDC42. GTPase-activating proteins (GAPs) bind activated forms of Rho GTPases and stimulate GTP hydrolysis. Through this catalytic function, Rho GAPs negatively regulate Rho-mediated signals. GAPs may also serve as effector molecules and play a role in signaling downstream of Rho and other Ras-like GTPases.

By screening a Jurkat cDNA library using a yeast 2-hybrid system with an activated form of RAC as bait, followed by screening a placenta cDNA library, Toure et al. (1998) isolated a cDNA encoding RACGAP1, which they called MGCRACGAP. The predicted 527-amino acid RACGAP1 protein has a large N-terminal region containing a protein kinase C-like cysteine-rich motif. RACGAP1 shares highest homology with the Drosophila RnRacGAP and the chimerins of rat and human. Functional analysis showed that the GAP domain of RACGAP1 exhibits strong GAP activity towards CDC42, RAC1, and RAC2. Northern blot analysis detected an approximately 3.2-kb RACGAP1 transcript that was most abundantly expressed in testis, with low expression in most other tissues. Western blot analysis detected a RACGAP1 protein of 58 kD in testis extracts. In situ hybridization showed that RACGAP1 expression is restricted to germ cells in mature testis. Human breakpoint cluster region (bcr) gene product is a member of a group of GTPase-activating proteins that act exclusively on members of the Ras-related Rho subfamily. A complementary DNA was isolated from Caenorhabditis elegans that encoded a polypeptide of 1438 amino acid residues, CeGAP, which contains a domain with sequence similarity to the COOH-terminal segment (GTPase-activating protein region) of Bcr and other known GTPase-activating proteins of the Rho subfamily. It also contains a “pleckstrin homology” motif, present in many signaling proteins including GTPase-activating proteins and nucleotide exchange factors. The Bcr-like domain of CeGAP exhibited activity not only on members of the C. elegans and human Rho subfamily but surprisingly also on C. elegans Ras protein (let-60), human Ras, and Rab3A.

CeGAP is therefore the first GTPase-activating protein acting on Ras-related proteins across different subfamilies. Together with the presence of the pleckstrin homology motif, this suggests a central and integrative role for CeGAP in a signaling pathway common to Ras and related proteins.

NOV85 is predicted to be expressed in at least the following tissues: pancreas, stomach, brain, bone. This information was derived by determining the tissue sources of the sequences that were included in the invention including but not limited to SeqCalling sources, public EST sources, literature sources, and/or RACE sources. Further expression data for NOV85 is provided in Example 2.

The NOV85 nucleic acids and proteins are useful in potential therapeutic applications implicated in various pathological disorders described further herein, for example, Alzheimer's disease, stroke, tuberous sclerosis, hypercalceimia, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, neuroprotection, hypercalceimia, ulcers, diabetes, Von Hippel-Lindau (VHL) syndrome, pancreatitis, obesity as well as other diseases, disorders and conditions. NOV85 nucleic acids encoding the CeGAP-like protein of the invention, or fragments thereof, may further be useful in diagnostic applications, wherein the presence or amount of the nucleic acid or the protein are to be assessed.

The novel nucleic acid of the invention encoding a GTPase activating protein-like protein includes the nucleic acid whose sequence is provided in Table 85A, or a fragment thereof. The invention also includes a mutant or variant nucleic acid any of whose bases may be changed from the corresponding base shown in Table 85A while still encoding a protein that maintains its GTPase activating protein-like activities and physiological functions, or a fragment of such a nucleic acid. The invention further includes nucleic acids whose sequences are complementary to the sequence of Table 85A, including nucleic acid fragments that are complementary to any of the nucleic acids just described.

The invention additionally includes nucleic acids or nucleic acid fragments, or complements thereto, whose structures include chemical modifications. Such modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 1% of the bases may be so changed.

The novel protein of the invention includes the GTPase activating protein-like protein whose sequence is provided in Table 85B. The invention also includes a mutant or variant protein any of whose residues may be changed from the corresponding residue shown in Table 85B while still encoding a protein that maintains its GTPase activating protein-like activities and physiological functions, or a functional fragment thereof. In the mutant or variant protein, up to about 1% of the amino acid residues may be so changed.

These materials are further useful in the generation of antibodies that bind immunospecifically to the substances of the invention for use in therapeutic or diagnostic methods. These antibodies may be generated according to methods known in the art, using prediction from hydrophobicity charts, as described in the “Anti-NOVX Antibodies” section below.

NOV86

The disclosed NOV86 (alternatively referred to herein as CG56957-01) includes the 3451 nucleotide sequence (SEQ ID NO:279) shown in Table 86A. A NOV86 ORF begins with a Kozak consensus ATG initiation codon at nucleotides 35-37 and ends with a stop codon at nucleotides 3442-3445.

TABLE 86A
NOV86 Nucleotide Sequence

(SEQ ID NO:279)

CCCGCCCCCTCGGGCTCCCGGCCGGGGCCCCATCATGTTCTCCAGGAAGAAACGAGAGCTCATGAAAACC
CCTTCCATCTCGAAAAAQAACCGCGCGGGAAGCCCCAGCCCGCAGCCCTCGAAAAAGCTGCCCAGGAAGG
ATGGGGCTGACGCGGTGTTCCCCGGACCAAGCCTGGAGCCGCCCGCTGGGTCCTCCGGCGTCAAGGCCAC
AGGGACCCTCAAGCGGCCCACCAGCCTGAGCCGCCACGCCAGCGCGGCTGGCTTCCCCCTGTCGGGTGCT
GCCTCCTGGACACTGGGCCGGAGCCACCGGAGCCCACTGACAGCCGCCAGCCCGGGCGAGCTGCCCACCG
AGGGTGCCGGCCCGGACGTCGTCGAGGACATCTCCCATCTGCTGGCGGACGTGGCCCGCTTCGCTGAGGG
CCTTGAGAAACTTAAGGAGTGTGTGTTGCGTCACGACCTCCTTGAGGCCCGCCGCCCGCGGGCCCACGAG
TGCCTGGGTGAGGCTCTGCGTGTCATGCATCAGATCATCTCCAAGTACCCGCTGCTGAACACCGTGGAGA
CGCTCACCGCAGCCGGCACCCTCATTGCCAAGGTCAAAGCCTTCCATTATGAGAGCAACAATGATCTGGA
GAAACAGGAGTTCCAGAAGGCCCTGGAGACGATTCCTGTGGCCTTCAGTACCACAGTGTCCGAGTTCCTC
ATGGGTGAAGTGCACAGCAGCACCCTCCTAGCAGTGCCTCCTGGGGACTCGAGCCAGTCCATGGAAAGCC
TGTATGGACCGGGCAGTGAGGGCACGCCTCCCAGCCTGGATGACTGTGACGCCGGCTGCCTGCCCGCCGA
GGAGGTGGACGTGCTGCTACAGCGCTGTGAGGGGGGCGTGGATGCCGCACTGCTGTATGCCAAGAACATG
GCCAAGTACATGAAGGACCTCATCAGCTACCTGGAGAAGCGGACGACGCTGGAGATGGAGTTTGCCAAGG
GCCTGCAGAAGATCGCTCACAACTGCAGACAGAGCGTCATGCAGGAGCCCCACATGCCGCTCCTGTCCAT
CTACTCGCTGGCCCTCGAGCAGGACCTGGAGTTCGGCCACAGCATGGTGCAGGCAATGGGCACCTTGCAG
ACCCAGACCTTCATGCAGCCCCTGACCCTGCGGCGGCTTGAACACGAGAAGCGCAGGAAGGAGATCAAGG
AGGCCTGGCACCGTGCCCAGAGGAAGCTGCAAGAGGCGGAGTCCAACCTGCGCAAGGCCAAGCAGGGTTA
CGTGCAGCGCTGCGAGGACCACGACAAGCCTCGCTTCCTCGTGGCCAAGGCGGAAAAGGAGCAGGCTGGC
AGCGCGCCGGGAGCAGGCGGCACGGCCACCAAGACCCTGGACAAGCGGCGCCGGCTGGAGGAAAAGGCCA
AGAACAAGGCGGAGGAAGCTATGGCCACCTACCGCACCTGCGTGGCCGACGCGAAGACGCAGAAGCAGGA
GCTGGAGGATACCAAGGTGACGGCGCTGCGGCAGATCCAGGAGGTCATCCGGCAGAGCGACCAAACCATC
AAGTCGGCCACGATCTCCTACTACCAGATGATGCATATGCAGACGGCGCCGCTGCCCGTGCACTTCCAGA
TCCTGTGTGACAGCAGCAAGCTGTATGACCCAGGCCAGCAGTACGCCTCCCACGTGCGCCAGCTGCAGCG
GGACCAGGAGCCCGATGTGCACTACGACTTTGAGCCCCACGTCTCCCCCAACGCCTGGTCCCCCGTCATG
CGTGCCCGGAAGAGCAGCTTCAACGTGAGTGATGTGGCGCGGCCCCAGGCTGCCGGGAGCCCCCCAGAAG
AAGGCGGGTGCACTGAGGGCACACCTGCCAAGGACCACAGGGCCGGGCGAGGACACCAGGTTCACAAGTC
ATGGCCGCTCTCGATCTCAGACTCGGACAGTGGGCTGGACCCCGGCCCTGGCGCAGGGGACTTTAAGAAG
TTCGAGCGGACGTCATCCAGTGGTACCATGTCGTCCACGGAGGAGCTGGTGGACCCAGACGGTGGACCCG
GGGCTTCAGCCTTTGAGCAGGCTGACCTCAACGGCATGACCCCCGAGCTGCCGGTGGCCGTGCCCAGTGG
ACCGTTCCGCCACGAGGGGCTGTCCAAGGCGGCCCGTACTCACCGGCTCCGGAAGCTCCGCACGCCCGCC
AAGTGCCGCGAGTGCAACAGCTACGTCTACTTCCACGGTGCTGAGTGTGAAGAGTGCTGCCTGGCCTGCC
ACAAGAAATGTCTGGAGACGCTGGCCATACAGTGCGGGCACAAGAAGCTGCAGGGCCGCCTGCAGCTGTT
CGGCCAAAACTTCAGCCACGCGGCCCGCAGCGCCCCCGACGGCGTGCCCTTCATCGTCAAGAAGTGCGTC
TGCGAGATCGAGCGGCGGGCGCTGCGCACCAAGGGCATCTACCGGGTCAATGGGGTAAAGACACGCGTGG
AGAAGCTGTGCCAGGCCTTCGAGAACGGCAAGGAGCTGGTCGAGCTGTCGCAGGCCTCGCCCCACCACAT
CAGCAACGTCCTCAAGCTCTACCTGCGTCAGCTTCCCCAGCCGCTCATCTCCTTCCGCCTCTACCACGAG
CTCGTAGGGCTGGCCAAGGACAGCCTGAAGGCAGAGGCCGAGGCCAAGGCGGCGTCCCGGGGCCGGCAGG
ACGGCTCGGAGAGCGAGGCAGTGGCGGTGGCCCTGGCAGGTCGGCTGCGGCAGCTCCTGCGAAACCTGCC
GCCTGAGAACCGGGCCTCGCTGCAGTACCTGCTGCGTCACCTACGCAGGAACGTGGAGGTGGAGCAGGAC
AACAAGATGACCCCCGGCAACCTGGGCATCGTGTTCGGGCCCACGCTGCTTCGGCCACGGCCCACCGAGG
CCACCGTGTCCCTCTCCTCCCTGGTGGATTATCCCCATCAGGCCCGCGTCATCGAGACTCTCATCGTCCA
CTACGGCCTGGTCTTCGAGGAGGAGCCCGAGGAGACCCCCGGGGGCCAGGACGAGTCATCCAACCAGCGA
GCTGAGGTAGTCGTCCAGGTGCCGTACCTGCAGGCGGGCGAGGCGGTGGTCTACCCGCTGCAGGAGGCGG
CGGCGGACGGGTGCAGAGAATCCCGAGTTGTGTCCAACGATTCGGACTCGGACCTAGAGGAGGCCTCCGA
GCTGCTGTCCTCATCGGAGGCCAGTGCCCTGGGCCACCTCAGCTTCCTAAAGCAGCAGCAGAGCGAGGCC
AGCCTAGAGGTGGCTTCTGGCAGCCACAGCGGCAGTGAGGAGCAGCTGGAGGCCACAGCCCGAAAGGACG
GGGACGGGGACGAGGACGGCCCGGCCCAGCAGCTCTCAGGATTCAACACCAACCAGTCCAACAACGTGCT
GCAGGCCCCACTGCCCCCCATGAGGCTCCGTGGCGGGCGGATGACACTGGGCTCCTCCAGGGAAAGGCAG
CCCGAATTCGTGTGAGCTGGG

A NOV86 polypeptide (SEQ ID NO:280) encoded by SEQ ID NO:279 is 1136 amino acids in length and is presented using the one-letter amino acid code in Table 86B. The Psort profile for NOV86 predicts that this sequence is likely to be localized to the nucelus with a certainty of 0.9800.

TABLE 86B
NOV86 Polypeptide Sequence

| | |
|---|---|
| MFSRKKRELMKTPSISKKNRAGSPSPQPSGELPRKDGADAVFPGPSLEPPAGSSGVAATG | (SEQ ID NO:280) |
| TLKRPTSLSRHASAAGFPLSGAASWTLGRSHRSPLTAASPGELPTEGAGPDAAEDISHLL | |
| ADVARFAEGLEKLKECVLRDDLLEARRPRAHECLGEALRAAHQIISKYPLLNTVETLTAA | |
| GTLIAKVKAFHYESNNDLEKQEFEKALETIAVAFSSTVSEFLMGEVDSSTLLAVPPGDSS | |
| QSMESLYGPGSEGTPPSLDDCDAGCLPAEEVDVLLQRCEGGVDAALLYAKNNAKYMAALI | |
| SYLEKRTTLEMEFAKGLQKIAHNCRQSVMQEPHMPLLSIYSLALEQDLEFGHSMVQAVGT | |
| LQTQTFNQPLTLRRLEHEKRRKEIKEAWHRAQRKLQEAESNLRKAKQGYVQRCEDHDKAA | |
| FLVAKAEEEQAGSAPGAGGTATKTLDKRRRLEEEAKNKAEEAMATYRTCVADAKTQKQEL | |
| EDTKVTALRQIQEVIRQSDQTIKSATISYYQMMHMQTAPLPVHFQMLCESSKLYDPGQQY | |
| ASHVRQLQRDQEPDVHYDFEPHVSANAWSPVMRARKSSFNVSDVARPEAAGSPPEEGGCT | |
| EGTPAKDHRAGRGHQVHKSWPLSISDSDSGLDPGPGAGDFKKFERTSSSGTMSSTEELAA | |
| PDGGAGASAFEQADLNGMTPELPVAVPSCPFRHEGLSKAARTHRLRKLRTPAKCRECNSY | |
| VYFQGAECEECCLACHKKCLETLAIQCGHKKLQGRLQLFGQDFSHAARSAPDGVPFIVKK | |
| CVCEIERRALRTKGIYRVNGVKTRVEKLCQAFENGKELVELSQASPHDISNVLKLYLRQL | |
| PEPLISFRLYHELVGLAKDSLKAEAEAKAASRCRQDGSESEAVAVAAAGRLRELLRDLPP | |
| ENRASLQYLLRHLRRIVEVEQDNKMTPGNLGIVFGPTLLRPRPTEATVSLSSLAAYPHQA | |
| RVIETLIVHYGLVFEEEPEETPGGQDESSNQRAEVVVQVPYLEAGEAVYVPLQEAAAAGC | |
| RESRVVSNDSDSDLEEASELLSSSEASALGHLSFLEQQQSEASLEVASGSHSGSEEQLEA | |
| TAREDGDGDEDGPAQQLSGFNTNQSNNVLQAPLPPMRLRGGRMTLGSCRERQPEFV | |

A BLAST analysis of NOV86 was run against the proprietary PatP GENESEQ Protein Patent database. It was found, for example, that the amino acid sequence of NOV86 had high homology to other proteins as shown in Table 86C.

TABLE 86C
BLASTX results from PatP database for NOV86

| | | Smallest |
|---|---|---|
| | | Sum |
| | High | Probability |
| Sequences producing High-scoring Segment Pairs: | Score | P(N) |
| patp: AAU17313 Novel signal transduction | 2855 | 5.7e−299 |
| pathway protein | | |
| patp: AAW75995 GTPase activating protein | 1497 | 2.9e−153 |
| (GAP), PARG | | |
| patp: AAY90268 Human GTP-ase activating | 1497 | 2.9e−153 |
| polypeptide PARG | | |
| patp: AAU17459 Novel signal transduction | 1265 | 1.1e−128 |
| pathway protein | | |
| patp: AAU17460 Novel signal transduction | 1127 | 4.7e−114 |
| pathway protein | | |

In a search of sequence databases, it was found, for example, that the nucleic acid sequence of this invention has 3447 of 3451 bases (99%) identical to a gb:GENBANK-ID:D86976|acc:D86976.1 mRNA from Homo sapiens (Human mRNA for KIAA0223 gene). The full amino acid sequence of the protein of the invention was found to have 1134 of 1136 amino acid residues (99%) identical to, and 1135 of 1136 amino acid residues (99%) similar to, the 1165 amino acid residue ptnr:SPTREMBL-ACC:Q92619 protein from Homo sapiens (Human) (MYELOBLAST KIAA0223). NOV86 also has homology to the other proteins shown in the BLASTP data in Table 86D.

TABLE 86D
NOV86 BLASTP results

| Gene Index/ | | Length | Identity | Positive | |
|---|---|---|---|---|---|
| Identifier | Protein/Organism | (aa) | (%) | (%) | Expect |
| gi\|14765644\|ref\| | minor histocompatibility | 1136 | 1134/1136 | 1135/1136 | 0.0 |
| XP_037574.1\| | antigen HA-1 | | (99) | (99) | |
| (XM_037574) | [Homo sapiens] | | | | |
| gi\|1504026\|dbj\| | similar to C. elegans | 1165 | 1134/1136 | 1135/1136 | 0.0 |
| BAA13212.1\| | protein (Z37093) [Homo | | (99) | (99) | |
| (D86976) | sapiens] | | | | |
| gi\|2896796\|gb\| | D1013901 [Homo sapiens] | 996 | 994/996 | 995/996 | 0.0 |
| AAC03237.1\| | | | (99) | (99) | |
| (AC004151) | | | | | |
| gi\|12857707\|dbj\| | MYELOBLAST KIAA0223 | 523 | 423/542 | 460/542 | 0.0 |
| BAB31085.1\| | (FRAGMENT)˜putative [Mus | | (78) | (84) | |
| (AK018130) | musculus] | | | | |
| gi\|13635768\|ref\| | similar to PTPL1- | 1261 | 345/881 | 509/881 | e−160 |
| XP_017232.1\| | associated RhoGAP 1 (H. | | (39) | (57) | |
| (XM_017232) | sapiens) [Homo sapiens] | | | | |

This BLASTP data is displayed graphically in the ClustalW in Table 86E. A multiple sequence alignment is given, with the NOV86 protein being shown on line 1 in a ClustalW analysis comparing the protein of the invention with the related protein sequences shown in Table 86D.

[image]

[image]

[image]

[image]

Table 86F lists the domain description from DOMAIN analysis results against NOV86. This indicates that the NOV86 sequence has properties similar to those of other proteins known to contain this domain.

TABLE 86F
Domain Analysis of NOV86

gn1|Smart|smart00324, RhoGAP, GTPase-activator protein for Rho-like GTPases;
GTPase activator proteins towards RhoIRacICdc42-Iike small GTPases
SEQ ID NO:879
CD-Length = 175 residues, 98.3% aligned
Score = 157 bits (396), Expect = 4e−39

| | | | |
|---|---|---|---|
| NOV86: | 774 | VPFIVKKCVCEIERRLRTKGIYRAAGVKTRAAKLCQAFENGKELVELSQASPAAISAA | 832 |
| | | +P IV+KC+ +E+R L T+GIYR +G +RV++L +AF++G + +LS+ HD++ + | |
| Sbjct: | 3 | IPIIVEKCIEYLEKRGLDTEGIYRKSGSASRVKELREAFDSGPDPDLDLSEYDAADVAGL | 62 |
| NOV86: | 833 | LKLYLRQLPEPLISFRLYHELVCLAKDSLKAEAEAKAASRGRQDGSESEAVAVAAAGRLR | 892 |
| | | LKL+LR+LPEPLI+R LY E + A + E E + | |
| Sbjct: | 63 | LKLFLRELPEPLITFELYEEFIEAA----------------KLEDEEERLRALREAAAA | 101 |
| NOV86: | 893 | ELLRDLPPENRASLQYLLRHLRRIVEVEQDNKMTPGNLGIVFGPTLLRPRPTEATVSLSS | 952 |
| | | LL LPP NRA+L+YLL HL R+ E ++NKMT NL IVFGPTLLRP E +S | |
| Sbjct: | 102 | AALLSLLPPARATLRYLLAHLNRVAEHSEENKMTARNLAIVFGPTLLRPPDGE----SAS | 156 |
| NOV86: | 953 | LVDYPHQARVIETLIVHY | 970 |
| | | L D HQ +V+E LI + | |
| Sbjct: | 157 | LKDIRHQNKVVEFLIENA 174 | |

Rho GTPases control a variety of cellular processes. There are 3 subtypes of Rho GTPases in the Ras superfamily of small G proteins: RHO, RAC, and CDC42. GTPase-activating proteins (GAPs) bind activated forms of Rho GTPases and stimulate GTP hydrolysis. Through this catalytic function, Rho GAPs negatively regulate Rho-mediated signals. GAPs may also serve as effector molecules and play a role in signaling downstream of Rho and other Ras-like GTPases.

By screening a Jurkat cDNA library using a yeast 2-hybrid system with an activated form of RAC as bait, followed by screening a placenta cDNA library, Toure et al. (1998) isolated a cDNA encoding RACGAP1, which they called MGCRACGAP. The predicted 527-amino acid RACGAP1 protein has a large N-terminal region containing a protein kinase C-like cysteine-rich motif. RACGAP1 shares highest homology with the Drosophila RnRacGAP and the chimerins of rat and human. Functional analysis showed that the GAP domain of RACGAP1 exhibits strong GAP activity towards CDC42, RAC1, and RAC2. Northern blot analysis detected an approximately 3.2-kb RACGAP1 transcript that was most abundantly expressed in testis, with low expression in most other tissues. Western blot analysis detected a RACGAP1 protein of 58 kD in testis extracts. In situ hybridization showed that RACGAP1 expression is restricted to germ cells in mature testis Human breakpoint cluster region (bcr) gene product is a member of a group of GTPase-activating proteins that act exclusively on members of the Ras-related Rho subfamily.

A complementary DNA was isolated from Caenorhabditis elegans that encoded a polypeptide of 1438 amino acid residues, CeGAP, which contains a domain with sequence similarity to the COOH-terminal segment (GTPase-activating protein region) of Bcr and other known GTPase-activating proteins of the Rho subfamily. It also contains a “pleckstrin homology” motif, present in many signaling proteins including GTPase-activating proteins and nucleotide exchange factors. The Bcr-like domain of CeGAP exhibited activity not only on members of the C. elegans and human Rho subfamily but surprisingly also on C. elegans Ras protein (let-60), human Ras, and Rab3A.

CeGAP is therefore the first GTPase-activating protein acting on Ras-related proteins across different subfamilies. studies suggest a central and integrative role for CeGAP in a signaling pathway common to Ras and related proteins.

NOV86 is predicted to be expressed in at least the following tissues: pancreas, stomach, brain, bone. This information was derived by determining the tissue sources of the sequences that were included in the invention including but not limited to SeqCalling sources, public EST sources, literature sources, and/or RACE sources. Further expression data for NOV86 is provided in Example 2.

The NOV86 nucleic acids and proteins are useful in potential therapeutic applications implicated in various pathological disorders described further herein, for example, Alzheimer's disease, stroke, tuberous sclerosis, hypercalceimia, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, neuroprotection, hypercalceimia, ulcers, diabetes, Von Hippel-Lindau (VHL) syndrome, pancreatitis, obesity as well as other diseases, disorders and conditions. NOV86 nucleic acids encoding the CeGAP-like protein of the invention, or fragments thereof, may further be useful in diagnostic applications, wherein the presence or amount of the nucleic acid or the protein are to be assessed.

The novel nucleic acid of the invention encoding a GTPase activating protein-like protein includes the nucleic acid whose sequence is provided in Table 86A, or a fragment thereof. The invention also includes a mutant or variant nucleic acid any of whose bases may be changed from the corresponding base shown in Table 86A while still encoding a protein that maintains its GTPase activating protein-like activities and physiological functions, or a fragment of such a nucleic acid. The invention further includes nucleic acids whose sequences are complementary to the sequence of Table 86A, including nucleic acid fragments that are complementary to any of the nucleic acids just described.

The invention additionally includes nucleic acids or nucleic acid fragments, or complements thereto, whose structures include chemical modifications. Such modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 1% of the bases may be so changed.

The novel protein of the invention includes the GTPase activating protein-like protein whose sequence is provided in Table 86B. The invention also includes a mutant or variant protein any of whose residues may be changed from the corresponding residue shown in Table 86B while still encoding a protein that maintains its GTPase activating protein-like activities and physiological functions, or a functional fragment thereof. In the mutant or variant protein, up to about 1% of the amino acid residues may be so changed.

These materials are further useful in the generation of antibodies that bind immunospecifically to the substances of the invention for use in therapeutic or diagnostic methods. These antibodies may be generated according to methods known in the art, using prediction from hydrophobicity charts, as described in the “Anti-NOVX Antibodies” section below.

NOV87

NOV87 includes two GTPase activating-like proteins, designated herein as NOV87a and NOV87b.

NOV87a

The disclosed NOV87a (alternatively referred to herein as CG56886-01) includes the 994 nucleotide sequence (SEQ ID NO:281) shown in Table 87A. A NOV87a ORF begins with a Kozak consensus ATG initiation codon at nucleotides 16-18 and ends with a TGA codon at nucleotides 982-984. The disclosed NOV87a maps to human chromosome 17.

TABLE 87A
NOV87a Nucleotide Sequence

(SEQ ID NO:281)

GGGGGCCACTCTCTCATGGCCCCCAAAGACAAATCCAGTAGGAAGAATGTCCTGGAGGTGAGTGGCGGAA
TTGGGGAGAAGGAGGGAAGGGGCTTAGTGATGCCTGCCAGCTCTCTGACAACTATTGACCTGGAAATGAC
CTGGGGGTTTTTGACCCTTAATTCCCAGCTACGGAGCCGAGATGGCTCTGAGTACCTGATCCAGCACGAC
TCGGAGGCCATCATCAGCACCTGGCATAGGCCATTGCTCAGGGCATCCAGGAGCTGGTAAAACAGAGCCC
AGGGCCTCAGCGACTTGAGCAAGGTCCGGCACAAGCTCCGCAAGTTCCTCCAGAGGCGGCCCACACTGCA
GTCGCTGCGGGAGAAGGGCTACATCAAAGACCAGGTGTTCGGCTGCGCGCTGGCCGCGCTGTGTGAGCGC
GAGAGGAGCCGGGTGCCACGCTTCGTGCAGCAGTGCATCCGCGCCGTCGAGGCCCGCGGTCTGGACATCG
ACGGGCTGTACCGCATCAGTGGAAACCTGGCCACCATCCAGAAGCTACGCTATAAGGTGGACCACGGTGA
GGATGAGCGCCTTGACCTGGATGACGGGCGCTCGGAGGACGTCCACGTTATCACCGGAGCCCTGAAGCTC
TTCTTTCGCGAOCTGCCCGAGCCCCTCTTCCCCTTCTCGCACTTCCGCCAGTTCATTGCGGCCATCAGTG
AGCAGGACCAGGCCCGGCGCAGCCGCTGTGTGCGTGACTTGGTGCGCTCGCTGCCCGCTCCCAACCACGA
CACTCTGCGGATGCTCTTCCAGCACCTCTGCCGGAGGGTGATCGAGCACGGCGAGCAGAACCCCATGTCG
GTGCAGAGCGTGGCCATTGTGTTCGGGCCCACGCTGCTGCGGCCCGAGGTGGAAGAGACCAGCATGCCCA
TGACCATGGTGTTCCAGAACCAGGTGGTGGAGCTCATCCTGCAGCAGTGCGCGGACATCTTCCCGCCGCA
CTGACTGCTGGCCT

A NOV87a polypeptide (SEQ ID NO:282) encoded by SEQ ID NO:281 is 322 amino acids in length and is presented using the one-letter amino acid code in Table 87B. The Psort profile for NOV87a predicts that this sequence is likely to be localized to the cytoplasm with a certainty of 0.6500.

TABLE 87B
NOV87a Polypeptide Sequence

| | |
|---|---|
| MAPKDKSSRKNVLEVSGGVGEKEGRGLVMPASSLTTTDLEMTWGFLTLNSQLRSRDGSEY | (SEQ ID NO:282) |
| LIQHDSEAIISTWHKAIAQGIQELVSRAQGLSDLSKVRHKLRKFLQRRPTLQSLREKGYI | |
| KDQVFGCALAALCERERSRVPRFVQQCIRAVEARGLDIDGLYRISGNLATIQKLRYKVDH | |
| GEDERLDLDDGRWEDVHVITGALKLFFRELPEPLFPFSHFRQFIAAISEQDQARRSRCVR | |
| DLVRSLPAPNHDTLRMLFQHLCRRVIEHGEQNRNSVQSVAIVFGPTLLRPEVEETSMPMT | |
| MVFQNQVVELILQQCADIFPPH | |

NOV87b

The disclosed NOV87b (alternatively referred to herein as CG56886-02) includes the 985 nucleotide sequence (SEQ ID NO:283) shown in Table 87C. A SEC2 ORF begins with a Kozak consensus ATG initiation codon at nucleotides 7-9 and ends with a TGA codon at nucleotides 973-975. The disclosed NOV87b maps to human chromosome 17.

TABLE 87C
NOV87b Nucleotide Sequence

(SEQ ID NO:283)

GGGGGCCACTCTCTCATGGCCCCCAAAGACAAATCCAGTAGGAAGAATGTGCTGGAGGTGAGTGGCGGGG
TTGGGGAGAAGGAGGGAAGGGGCTTAGTGATGCCTGCCAGCTCTCTGACAACTATTGACCTGGAAATGAC
CTGGGGGTTTTTGACCCTTAATTCCCAGCTACGGAGCCGAGATGGCTCTGAGTACCTGATCCAGCACGAC
TCGGAGGCCATCATCAGCACCTGGCATAAGGCCATTGCTCAGGGCATCCAGGAGCTGGTAACCAGAGCCC
AGGGCCTCAGCGACTTGAGCAAGGTCCGGCACAAGCTCCGCAAGTTCCTCCAGAGGCGGCCCACACTGCA
GTCGCTGCGGGAGAAGGGCTACATCAAAGACCACGTGTTCGGCTGCGCGCTGGCCGCGCTGTGTGAGCGC
GAGAGGAGCCGGGTGCCACGCTTCGTGCAGCAGTGCATCCGCGCCGTCGAGGCCCGCGGTCTGGACATCG
ACGGGCTGTACCGCATCAGTGGAAACCTGGCCACCATCCAGAAGCTACGCTATAAGGTGGACCACGCTGA
GGATGAGCGCCTTGACCTGGATGACCGGCGCTGGCAGGACGTCCACGTTATCACCGGAGCCCTGAAGCTC
TTCTTTCGGGACCTGCCCGAGCCCCTCTTCCCCTTCTCGCACTTCCOCCAGTTCATTGCGGCCATCAGTG
AGCAGGACCAGGCCCGGCGCAGCCGCTGTGTGCGTGACTTGGTGCGCTCGCTGCCCGCTCCCAACCACGA
CACTCTGCGGATGCTCTTCCAGCACCTCTGCCGGAGGGTGATCGAGCACGCCGAGCAGAACCGCATGTCG
GTGCAGAGCGTGGCCATTGTGTTCGGGCCCACGCTGCTGCGGCCCGAGGTGGAAGAGACCAGCATGCCCA
TGACCATGGTGTTCCAGAACCAGGTGGTGGAGCTCATCCTGCAGCAGTGCGCGGACATCTTCCCGCCGCA
CTGACTGCTGGCCT

The NOV87b polypeptide (SEQ ID NO:284) encoded by SEQ ID NO:283 is 322 amino acids in length and is presented using the one-letter amino acid code in Table 87D. The Psort profile for NOV87b predicts that this sequence has no signal peptide and is likely to be localized to the cytoplasm with a certainty of 0.6500.

TABLE 87D
NOV87b Polypeptide Sequence

| | |
|---|---|
| MAPKDKSSRKNVLEVSGGVGEKEGRGLVMPASSLTTIDLEMTWGFLTLNSQLRSRDGSEY | (SEQ ID NO:284) |
| LIQHDSEAIISTWHKAIAQGIQELVSRAQGLSDLSKVRHKLRKFLQRRPTLQSLREKGYI | |
| KDQVFGCALAALCERERSRVPRFVQQCIRAVEARGLDIDGLYRISGNLATIQKLRYKVDH | |
| GEDERLDLDDGRWEDVHVITGALKLFFRELPEPLFPFSHFRQFIAAISEQDQARRSRCVR | |
| DLVRSLPAPNHDTLRMLFQHLCRRVIEHGEQNRMSVQSVAIVFGPTLLRPEVEETSMPMT | |
| MVFQNQVVELILQQCADIFPPH | |

A BLAST analysis of NOV87 was run against the proprietary PatP GENESEQ Protein Patent database. It was found, for example, that the amino acid sequence of NOV87 had high homology to other proteins as shown in Table 87E.

TABLE 87E
BLASTX results from PatP database for NOV87

| | | Smallest |
|---|---|---|
| | | Sum |
| | High | Probability |
| Sequences producing High-scoring Segment Pairs: | Score | P(N) |
| patp: AAU17449 Novel signal transduction | 827 | 2.9e−82 |
| pathway protein | | |
| patp: AAB68548 Human GTP-binding | 779 | 3.5e−77 |
| associated protein | | |
| patp: AAG66505 GTP enzyme Rho family | 779 | 3.5e−77 |
| active site 90 | | |
| patp: AAB64387 Amino acid sequence of | 711 | 5.6e−70 |
| human intracellular | | |
| patp: AAY94450 Human inflammation associated | 589 | 3.0e−62 |
| protein | | |

In a search of sequence databases, it was found, for example, that the nucleic acid sequence of this invention has 276 of 417 bases (66%) identical to a gb:GENBANK-ID:BC006107|acc:BC006107.1 mRNA from Homo sapiens (Homo sapiens, clone MGC:12959, mRNA). The full amino acid sequence of the protein of the invention was found to have 173 of 319 amino acid residues (54%) identical to, and 229 of 319 amino acid residues (71%) similar to, the 316 amino acid residue ptnr:SPTREMBL-ACC:Q9NT76 protein from Homo sapiens (Human) (HYPOTHETICAL 36.4 KDA PROTEIN). NOV87 also has homology to the other proteins shown in the BLASTP data in Table 87F.

TABLE 87F
NOV87 BLASTP results

| Gene Index/ | | Length | Identity | Positive | |
|---|---|---|---|---|---|
| Identifier | Protein/Organism | (aa) | (%) | (%) | Expect |
| gi\|113360091\|pir\|\| | hypothetical protein | 316 | 168/355 | 221/355 | 7e−80 |
| T46471 | DKFZp434L0130.1 - human | | (47) | (61) | |
| gi\|13676443\|dbj\| | hypothetical protein | 847 | 164/356 | 220/356 | 6e−79 |
| BAB41146.1\| | [Macaca fascicularis] | | (46) | (61) | |
| (AB060206) | | | | | |
| gi\|14245732\|dbj\| | rho-GTPase activating | 731 | 130/328 | 188/328 | 9e−57 |
| BAB56159.1\| | protein [Homo sapiens] | | (39) | (56) | |
| (AB051853) | | | | | |
| gi\|18146831\|dbj\| | RGL1 [Homo sapiens] | 547 | 130/328 | 188/328 | 2e−56 |
| BAB83128.1\| | | | (39) | (56) | |
| (AB030239) | | | | | |
| gi\|15080081\|gb\| | Unknown (protein for | 599 | 130/328 | 188/328 | 2e−56 |
| AAH11820.1\| | IMAGE: 3619501) | | (39) | (56) | |
| AAH11820 | [Homo sapiens] | | | | |
| (BC011820) | | | | | |

This BLASTP data is displayed graphically in the ClustalW in Table 87G. A multiple sequence alignment is given, with the NOV87a and b proteins being shown on lines 1 and 2 in a ClustalW analysis comparing the protein of the invention with the related protein sequences shown in Table 87F.

[image]

[image]

[image]

[image]

Table 87H lists the domain description from DOMAIN analysis results against NOV87. This indicates that the NOV87 sequence has properties similar to those of other proteins known to contain this domain.

TABLE 87H
Domain Analysis of NOV87

gnl|Smart|smart00324, RhoGAP, GTPase-activator protein for Rho-like GTPases;
GTPase activator proteins towards Rho/Rac/Cdc42-like small GTPases
SEQ ID NO:879
CD-Length = 175 residues, 99.4% aligned
Score = 144 bits (364), Expect = 6e−36

| | | | |
|---|---|---|---|
| NOV87: | 138 | SRVPRFVQQCIRAVEARGLDIDGLYRISGNLATIQKLRYKVDHGEDERLDLDDGRWEDVH | 197 |
| | | +P V++CI +E RGLD +G+YR SG+ + +++LR D G D LDL + DVH | |
| Sbjct: | 1 | KPIPIIVEKCIEYLEKRGLDTEGIYRKSGSASRVKELREAFDSGPDPDLDLSE---YDVH | 57 |
| | | ∘∘ | |
| NOV87: | 198 | VITGALKLFFRELPEPLFPFSHFRQFIAAISEQDQARRSRCVRDLVRSLPAPNHDTLRML | 257 |
| | | + G LKLF RELPEPL F + +FI A +D+ R R +R+L+ LP N TLR L | |
| Sbjct: | 58 | DVAGLLKLFLRELPEPLITFELYEEFIEAAKLEDEEERLRALRELLSLLPPANRATLRYL | 117 |
| | | ∘ | |
| NOV87: | 258 | FQHLCRRVIEHGEQNRMSVQSVAIVFGPTLLRPEVEETSMPMTMVFQNQVVELILQQC | 315 |
| | | HL RV EH E+N+M+ +++AIVFGPTLLRP E++ QN+VVE +++ | |
| Sbjct: | 118 | LAHL-NRVAEHSEENKMTARNLAIVFGPTLLRPPDGESASLKDIRHQNKVVEFLIENA | 174 |

Rho GTPases control a variety of cellular processes. There are 3 subtypes of Rho GTPases in the Ras superfamily of small G proteins: RHO, RAC, and CDC42. GTPase-activating proteins (GAPs) bind activated forms of Rho GTPases and stimulate GTP hydrolysis. Through this catalytic function, Rho GAPs negatively regulate Rho-mediated signals. GAPs may also serve as effector molecules and play a role in signaling downstream of Rho and other Ras-like GTPases.

By screening a Jurkat cDNA library using a yeast 2-hybrid system with an activated form of RAC as bait, followed by screening a placenta cDNA library, Toure et al. (1998) isolated a cDNA encoding RACGAP1, which they called MGCRACGAP. The predicted 527-amino acid RACGAP1 protein has a large N-terminal region containing a protein kinase C-like cysteine-rich motif. RACGAP1 shares highest homology with the Drosophila RnRacGAP and the chimerins of rat and human. Functional analysis showed that the GAP domain of RACGAP1 exhibits strong GAP activity towards CDC42, RAC1, and RAC2. Northern blot analysis detected an approximately 3.2-kb RACGAP1 transcript that was most abundantly expressed in testis, with low expression in most other tissues. Western blot analysis detected a RACGAP1 protein of 58 kD in testis extracts. In situ hybridization showed that RACGAP1 expression is restricted to germ cells in mature testis Human breakpoint cluster region (bcr) gene product is a member of a group of GTPase-activating proteins that act exclusively on members of the Ras-related Rho subfamily.

A complementary DNA was isolated from Caenorhabditis elegans that encoded a polypeptide of 1438 amino acid residues, CeGAP, which contains a domain with sequence similarity to the COOH-terminal segment (GTPase-activating protein region) of Bcr and other known GTPase-activating proteins of the Rho subfamily. It also contains a “pleckstrin homology” motif, present in many signaling proteins including GTPase-activating proteins and nucleotide exchange factors. The Bcr-like domain of CeGAP exhibited activity not only on members of the C. elegans and human Rho subfamily but surprisingly also on C. elegans Ras protein (let-60), human Ras, and Rab3A.

CeGAP is therefore the first GTPase-activating protein acting on Ras-related proteins across different subfamilies. studies suggest a central and integrative role for CeGAP in a signaling pathway common to Ras and related proteins.

NOV87 is predicted to be expressed in at least the following tissues: pancreas, stomach, brain, bone. This information was derived by determining the tissue sources of the sequences that were included in the invention including but not limited to SeqCalling sources, public EST sources, literature sources, and/or RACE sources. Further expression data for NOV87 is provided in Example 2.

The NOV87 nucleic acids and proteins are useful in potential therapeutic applications implicated in various pathological disorders described further herein, for example, Alzheimer's disease, stroke, tuberous sclerosis, hypercalceimia, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, neuroprotection, hypercalceimia, ulcers, diabetes, Von Hippel-Lindau (VHL) syndrome, pancreatitis, obesity as well as other diseases, disorders and conditions. NOV87 nucleic acids encoding the CeGAP-like protein of the invention, or fragments thereof, may further be useful in diagnostic applications, wherein the presence or amount of the nucleic acid or the protein are to be assessed.

The novel nucleic acid of the invention encoding a GTPase activating protein-like protein includes the nucleic acid whose sequence is provided in Table 87A or 96C, or a fragment thereof. The invention also includes a mutant or variant nucleic acid any of whose bases may be changed from the corresponding base shown in Table 87A or 96C while still encoding a protein that maintains its GTPase activating protein-like activities and physiological functions, or a fragment of such a nucleic acid. The invention further includes nucleic acids whose sequences are complementary to the sequence of Table 87A or 96C, including nucleic acid fragments that are complementary to any of the nucleic acids just described.

The invention additionally includes nucleic acids or nucleic acid fragments, or complements thereto, whose structures include chemical modifications. Such modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 1% of the bases may be so changed.

The novel protein of the invention includes the GTPase activating protein-like protein whose sequence is provided in Table 87B or 96D. The invention also includes a mutant or variant protein any of whose residues may be changed from the corresponding residue shown in Table 87B or 96D while still encoding a protein that maintains its GTPase activating protein-like activities and physiological functions, or a functional fragment thereof. In the mutant or variant protein, up to about 1% of the amino acid residues may be so changed.

These materials are further useful in the generation of antibodies that bind immunospecifically to the substances of the invention for use in therapeutic or diagnostic methods. These antibodies may be generated according to methods known in the art, using prediction from hydrophobicity charts, as described in the “Anti-NOVX Antibodies” section below.

NOV88

The disclosed NOV88 (alternatively referred to herein as CG56394-01) includes the 1092 nucleotide sequence (SEQ ID NO:285) shown in Table 88A. A NOV88 ORF begins with a Kozak consensus ATG initiation codon at nucleotides 25-27 and ends with a stop codon at nucleotides 1033-1035. The disclosed NOV88 maps to human chromosome 2.

TABLE 88A
NOV88 Nucleotide Sequence

(SEQ ID NO:285)

GCACCAGCCACATCCTGAGATACCATGGTTAAGGTGAAGGCCAGAGTCAACAGATTTGGCCACATTGGGC
ACCAGATCACCAGGGCTGCTTTTAACTCTGGTAAAGTGGATATTGTTGCCATCAGTGACCCCTTCACTGG
CCTCAACTACATGGTCTACGTGTTCCAGTGTGGTTCTACCCATGGCAAATTCCATGGCACTGTCAAGGCT
GAGAATGGGAAGCTTGTCATTAACGGAAATCTCATCACCATCTTTCAGGAGCGAGATCCCACCAAAATCA
AATGGGACAATGTTGACGCTGAGTACATTTGGGTGTCCACCGGTGTCTTCACCACCACAGAGAAGGCTGG
GGCTCACTTCCAGCAGGGAGCCAAAAGGGTCATAATCTCTACTCCCTCTGCTGACGCCCCCATGTTCATG
ATGGGCGTGAACCATAAGAAATATGAAAACAGCCTCAAGATCATCAGCAATGCCTCCTGTACCACCAACT
TCTTAGCCTCCCTGGCCAAGCTCATCCATGACAACTTTGGTATTGTGGAAGGACTCATGACCACGACCCA
CACCATCACTGCCACCCAGAAGACTGTAGATGGACCCTCCAGGAAACTGTGGTGTGATGGCCACGGGGCT
CTCCAGATCATCATCCCTGCATCTACTGGTGCTGCCAAAGCTGTAGGCAAGGTCATCCCCGAGATGAATG
GGAAGATTACTAGCATGGCCTTCCGTGTCCCCACCACCAATGTGTCGGTCATGCATCTGACCTGCCATCT
GGAAAATCCTGCCAAATATGATGACATCAAGAAGGTGGTGAAACAGGCATCAGAGGCCCCTCCCCTCAAG
GGCATCCTGGACTACACTGAGCACCACGTTGTCTCCTCCAGCTTTAACAGTGACACCCACTCTTCCACCT
TCAATGATGGGGCTGGTATTGCCCTCAATGACCATTTTGTCAAGCTCATTTCCTGTTATGACAATGCATT
TGGCTACAACAACAGGGCAGTGGACCTCATGGCCCACATGGCCTCCAAGAAGTAAGACCCCCAGACCACC
AGCCTCAGGCCCTCAGCTGCTAGGAATCCCCTATTGCACTAG

A NOV88 polypeptide (SEQ ID NO:286) encoded by SEQ ID NO:285 is 336 amino acids in length and is presented using the one-letter amino acid code in Table 88B. The Psort profile for NOV88 predicts that this sequence has no signal peptide and is likely to be localized to microbodies with a certainty of 0.4804. In alternative embodiments, a NOV88 polypeptide is located to the mitochondrial matrix space with a certainty of 0.3600.

TABLE 88B
NOV88 Polypeptide Sequence

| | |
|---|---|
| MVKVKARVNRFGHIGHQITRAAFNSGKVDIVAISDPFTGLNYMVYVFQCGSTHGKFHGTV | (SEQ ID NO:286) |
| KAENGKLVINGNLITIFQERDPTKIKWDNVDAEYIWVSTGVFTTTEKAGAHLQQGAKRVI | |
| ISTPSADAPMFMMGVNHKKYENSLKIISNASCTTNFLASLAKLIHDNFGIVEGLMTTTHT | |
| ITATQKTVDGPSRKLWCDGHGALQIIIPASTGAAKAVGKVIPEMNGKITSMAFRVPTTNV | |
| SVMHLTCHLENPAKYDDIKKVVKQASEAPPLKGILDYTEHHVVSSSFNSDTHSSTFNDGA | |
| GIALNDHFVKLISCYDNAFGYNNRAVDLMAHMASKK | |

A BLAST analysis of NOV88 was run against the proprietary PatP GENESEQ Protein Patent database. It was found, for example, that the amino acid sequence of NOV88 had high homology to other proteins as shown in Table 88C.

TABLE 88C
BLASTX results from PatP database for NOV88

| | | Smallest |
|---|---|---|
| | | Sum |
| | High | Probability |
| Sequences producing High-scoring Segment Pairs: | Score | P(N) |
| patp: AAY07036 Breast cancer associated | 1407 | 9.9e−144 |
| antigen precursor | | |
| patp: AAY05368 Human HCMV inducible | 1407 | 9.9e−144 |
| gene protein | | |
| patp: AAG64817 Human G3PDH fragment - | 1377 | 1.5e−140 |
| Homo sapiens, 327 aa. | | |
| patp: AAE04373 Mouse cancer associated | 1316 | 4.4e−134 |
| antigen OY-MC-2 | | |
| patp: AAR12995 GAP-DH - Aspergillus oryzae | 993 | 7.4e−100 |
| (ATCC 42149) | | |

In a search of sequence databases, it was found, for example, that the nucleic acid sequence of this invention has 928 of 1055 bases (87%) identical to a gb:GENBANK-ID:AF261085|acc:AF261085.1 mRNA from Homo sapiens (glyceraldehyde-3-phosphate dehydrogenase (GADPH) mRNA). The full amino acid sequence of the protein of the invention was found to have 278 of 336 amino acid residues (82%) identical to, and 294 of 336 amino acid residues (87%) similar to, the 335 amino acid residue ptnr:TREMBLNEW-ACC:AAG01996 protein from Homo sapiens (Human) (CLONE CDABP0047 MRNA SEQUENCE). NOV88 also has homology to the other proteins shown in the BLASTP data in Table 88D.

TABLE 88D
NOV88 BLASTP results

| Gene Index/ | | Length | Identity | Positive | |
|---|---|---|---|---|---|
| Identifier | Protein/Organism | (aa) | (%) | (%) | Expect |
| gi\|7669492\|ref\| | glyceraldehyde-3- | 335 | 278/336 | 294/336 | e−152 |
| NP_002037.2\| | phosphate dehydrogenase | | (82) | (86) | |
| (NM_002046) | [Homo sapiens] | | | | |
| gi\|31645\|emb\|CAA25833.1\| | glyceraldehyde-3- | 335 | 227/336 | 294/336 | e−152 |
| (X01677) | phosphate | | (82) | (87) | |
| | dehydrogenase | | | | |
| | [Homo sapiens] | | | | |
| gi\|2407184\|gb\|AAB94053.1\| | glyceraldehyde 3- | 333 | 272/333 | 290/333 | e−149 |
| (AF017079) | phosphate dehydrogenase | | (81) | (86) | |
| | [Sus scrofa] | | | | |
| gi\|6983849\|dbj\| | glyceraldehyde-3- | 333 | 267/333 | 285/333 | e−145 |
| BAA90818.1\| | phosphate dehydrogenase | | (80) | (85) | |
| (AB038241) | [Felis catus] | | | | |
| gi\|2506441\|sp\|P00355\| | GLYCERALDEHYDE 3- | 333 | 265/333 | 284/333 | e−145 |
| G3P_PIG | PHOSPHATE | | (79) | (84) | |
| | DEHYDROGENASE | | | | |
| | (GAPDH) | | | | |

This BLASTP data is displayed graphically in the ClustalW in Table 88E. A multiple sequence alignment is given, with the NOV88 protein being shown on line1 in a ClustalW analysis comparing the protein of the invention with the related protein sequences shown in Table 88D.

[image]

[image]

Table 88F lists the domain description from DOMAIN analysis results against NOV88. This indicates that the NOV88 sequence has properties similar to those of other proteins known to contain this domain.

TABLE 88F
Domain Analysis of NOV88

gnl|Pfam|pfam02800, gpdh_C, Glyceraldehyde 3-phosphate dehydrogenase, C-
terminal domain. GAPDH is a tetrameric NAD-binding enzyme involved in
glycolysis and glyconeogenesis. C-terminal domain is a mixed alpha/anti-
parallel beta fold. SEQ ID NO: 880
CD-Length = 163 residues, 100.0% aligned
Score = 169 bits (427), Expect = 3e−43

| | | | |
|---|---|---|---|
| NOV88: | 153 | TTNFLASLAKLIHDNFGIVEGLMTTTHTITATQKTVDGPSRKLWCDGHGALQIIIPASTG | 212 |
| | | TTN LA LAK+++DNFGI +GLMTT H TA QK VDGP K G A IIP STG | |
| Sbjct: | 1 | TTNCLAPLAKVLNDNFGIEKGLMTTVHAYTADQKLVDGPHHKDLRRGRAAAPNIIPTSTG | 60 |
| NOV88: | 213 | AAKAVGKVIPEMNGKITSMAFRVPTTNVSVMHLTCHLENPAKYDDIKKVVKQASEAPPLK | 272 |
| | | AAKAVG V+PE+NGK+T MAFRVPT NVSV+ LT LE P ++I +K+A+E P LK | |
| Sbjct: | 61 | AAKAVGLVLPELNGKLTGMAFRVPTPNVSVVDLTVELEKPVTVEEINAALKEAAEGPALK | 120 |
| NOV88: | 273 | GILDYTEHHVVSSSFNSDTHSSTFNDGAGIALNDHFVKLISCY 315 | |
| | | GIL YTE +VSS F D HSS F+ A I LND+FVKL++ Y | |
| Sbjct: | 121 | GILGYTEDPLVSSDFIGDPHSSIFDAKATIVLNDNFVKLVAWY 163 | |

NAD-dependent glycerol-3-phosphate dehydrogenase (EC 1.1.1.8) (GPD) catalyzes the reversible reduction of dihydroxyacetone phosphate to glycerol-3-phosphate. It is a cytoplasmic protein that is active as a homodimer, with each monomer containing an N-terminal NAD binding site. In insects, it acts in conjunction with a mitochondrial alpha-glycerophosphate oxidase in the alpha-glycerophosphate cycle, which is essential for the production of energy used in insect flight.

Glyceraldehyde-3-phosphate dehydrogenase (EC 1.2.2.12) (GAPDH) mRNA levels, protein, and enzymatic activity increase in 3T3-F442A adipocytes after exposure to physiological concentrations of insulin (Alexander, M., Curtis, G., Avruch, J., and Goodman, H. (1985) J. Biol. Chem. 260, 11978-11985). In order to understand the mechanism of this regulation, researchers isolated and sequenced 5.4 kilobase pairs of a 12-kilobase pair human genomic clone encoding a functional GAPDH gene. The gene consists of 9 exons and 8 introns with eukaryotic signals necessary for the transcription and translation of GAPDH mRNA. The exon sequence confirms previously published cDNA sequences for human GAPDH in muscle, liver, and erythrocytes. The organization of the human and the unique chicken GAPDH genes is strikingly similar. Although chicken exons VIII-XI have been fused into human exon 8, introns which separate exons encoding the NAD binding, catalytic, and helical domains of the GAPDH protein have been retained. Stable transfection of rodent cells with the intact human GAPDH gene resulted in the expression of a correctly initiated human GAPDH mRNA and an enzymatically active human GAPDH polypeptide. Thus, the gene contains a functional promoter and intact coding sequences. Although many processed GAPDH pseudogenes and GAPDH-like sequences are present in the human genome, Southern blot analysis of human genomic DNA using a probe derived from the 3′-untranslated region of the GAPDH gene detected only two genes, a 10-copy processed pseudogene and a single copy of the isolated gene. In contrast, a probe derived from an intron segment of the isolated gene detected only a single copy of the GAPDH gene. Collectively, these findings strongly suggest that the human genome encodes a single functional GAPDH gene.

Hopkinson et al. (1974) presented evidence that glycerol-3-phosphate dehydrogenase (EC 1.1.1.8) is a dimer of dissimilar subunits. Electrophoretic variants at each of two loci, designated GPD1 and GPD2, were described. By the method of somatic cell hybridization, Kielty and Povey (1982) assigned the presumed structural gene for alpha-glycerophosphate dehydrogenase to chromosome 12. Since this is a liver-specific enzyme, a rat hepatoma cell line was used as one of the ‘parents’ in the hybridization.

NOV88 is predicted to be expressed in at least the following tissues: liver. This information was derived by determining the tissue sources of the sequences that were included in the invention including but not limited to SeqCalling sources, public EST sources, literature sources, and/or RACE sources. Further expression data for NOV88 is provided in Example 2.

The NOV88 nucleic acids and proteins are useful in potential therapeutic applications implicated in various pathological disorders described further herein, for example, Von Hippel-Lindau (VHL) syndrome, cirrhosis, transplantation as well as other diseases, disorders and conditions. NOV88 nucleic acids encoding the Glycerol-3-Phosphate Dehydrogenase-like protein of the invention, or fragments thereof, may further be useful in diagnostic applications, wherein the presence or amount of the nucleic acid or the protein are to be assessed.

The novel nucleic acid of the invention encoding a Glycerol-3-Phosphate Dehydrogenase-like protein includes the nucleic acid whose sequence is provided in Table 88A, or a fragment thereof. The invention also includes a mutant or variant nucleic acid any of whose bases may be changed from the corresponding base shown in Table 88A while still encoding a protein that maintains its Glycerol-3-Phosphate Dehydrogenase-like activities and physiological functions, or a fragment of such a nucleic acid. The invention further includes nucleic acids whose sequences are complementary to the sequence of Table 88A, including nucleic acid fragments that are complementary to any of the nucleic acids just described.

The invention additionally includes nucleic acids or nucleic acid fragments, or complements thereto, whose structures include chemical modifications. Such modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 13% of the bases may be so changed.

The novel protein of the invention includes the Glycerol-3-Phosphate Dehydrogenase-like protein whose sequence is provided in Table 88B. The invention also includes a mutant or variant protein any of whose residues may be changed from the corresponding residue shown in Table 88B while still encoding a protein that maintains its Glycerol-3-Phosphate Dehydrogenase-like activities and physiological functions, or a functional fragment thereof. In the mutant or variant protein, up to about 18% of the amino acid residues may be so changed.

These materials are further useful in the generation of antibodies that bind immunospecifically to the substances of the invention for use in therapeutic or diagnostic methods. These antibodies may be generated according to methods known in the art, using prediction from hydrophobicity charts, as described in the “Anti-NOVX Antibodies” section below.

NOV89

The disclosed NOV89 (alternatively referred to herein as CG56396-01) includes the 1221 nucleotide sequence (SEQ ID NO:287) shown in Table 89A. A NOV89 ORF begins with a Kozak consensus ATG initiation codon at nucleotides 25-27 and ends with a stop codon at nucleotides 1033-1035. The disclosed NOV89 maps to human chromosome 6.

TABLE 89A
NOV89 Nucleotide Sequence

(SEQ ID NO:287)

CTGCATCTTCTCGTGCATTGCCAGCTGCATCCCTGAGACACCATGGTGAAGGTGAAGGCTGGAGTCAACA
GATTTGGTTGTATTGGCTGCCTGGTCACCAGGGCTGCTTTAAACTCTGGTTTAGTCGATATTGTCGCCAT
CAATGACCCCTTCATTGACCTCAACAACACTGTCTACATGTTCCAGTATAATTCCGCCCATGGCAAATTC
CACGGCACCGTCAAGGCTGAGAACGGGAAGCTTGTTATCAATGGAAATCTCATCACTATTTTCCAGGGGC
AAGATCTCACCAAAATCAAATGGGGCAATGCTGGCACTGAGTACATCATGGAGTTCACCAGCATCTTCAC
CACCATGGAGAAGGCTGGGGCTCACTTGGAGGGAGGAGCCAAAACGGTCATCATCTCTGCACCCTCTGCT
GATGCCCCCATGTTCGTGATGGGTGTGAACCATGAGAAATATGACAACAGCTCAAGATTACTCAAGATTA
TCAGCAATGCCTCCTGCACCACCAGCTGCTTAACGCCCCTGGCCAAGGTCATCCATGACAACTTTGGTAC
CGTGGAAGGACTCATGACCATCGCTGCCACCCAGAAGACTATGGATGGCTCCTATGGGAAACTGTGGGGT
GACGGCCATGGGGCTCTCCAGAACATCCTCTCTGCCTCTACTGGTGCTGCCAAGGCTGTGAGGAAGGTCA
TCCCTGAGCTAAACGGGAAGCTCACTGGCATGGCCTTCCGTGTCCCCACTGCCAACATGTCAGTGGTGGA
CCTGACCTGCCGTCTGGAAAAACCTACCAAATATGATGACACCAAGAAGGTGGTGAAGCAGGCGTCAGAG
GACCCCCTCAAAGGCATCCTGGGCTACTCTGAGCACCAGGTGGTCTCCTCCAACTTCAACTCAACAGACA
CCCACTCTTCCACCTTCGATGCTGGGGCTGGCATTGCCCTCAACGACCACTTTGTCAAGCTCATTTCCTG
GTATGACAATGAATTTGGCTGCAGCAACAGGGTGGTGGACCTCTGCCCACAGTGTGGCTTCCAAGGAGTA
AGACCCCCAGACCACCAGCCCCAGCGACAGCACGACGGGAAGAGAGCGGCCCTCACTGCTGGAGAGTCCC
TGCCACACTCAGTCTCCCACCACACTGAGAATCTCCCCTCCTCATAGTTTCCATGCAGACCCCCTAAAAG
GGAGGAGCCGAGGGAGCCCCACCTTTTCATG

A NOV89 polypeptide (SEQ ID NO:288) encoded by SEQ ID NO:287 is 374 amino acids in length and is presented using the one-letter amino acid code in Table 89B. The Psort profile for NOV89 predicts that this sequence is likely to be localized, to the endoplasmic reticulum (membrane) with a certainty of 0.5500, or to lysosomes with a certainty of 0.2630.

TABLE 89B
NOV89 Polypeptide Sequence

| | |
|---|---|
| MVKVKAGVNRFGCIGCLVTRAALNSGLVDIVAINDPFIDLNNTVYMFQYNSAHGKFHGTV | (SEQ ID NO:288) |
| KAENGKLVINGNLITIFQGQDLTKIKWGNAGTEYIMEFTSIFTTMEKAGAHLEGGAKTVI | |
| ISAPSADAPMFVMGVNHEKYDNSSRLLKIISNASCTTSCLTPLAKVIHDNFGTVEGLMTI | |
| AATQKTMDGSYGKLWGDGHGALQNILSASTGAAKAVRKVIPELNGKLTGMAFRVPTANMS | |
| VVDLTCRLEKPTKYDDTKKVVKQASEDPLKGILGYSEHQVVSSNFNSTDTHSSTFDAGAG | |
| IALNDHFVKLISWYDNEFGCSNRVVDLCPQCGFQGVRPPDHQPQRQHDGKRAALTAGESL | |
| PHSVSHHTENLPSS | |

A BLAST analysis of NOV89 was run against the proprietary PatP GENESEQ Protein Patent database. It was found, for example, that the amino acid sequence of NOV89 had high homology to other proteins as shown in Table 89C.

TABLE 89C
BLASTX results from PatP database for NOV89

| | | Smallest |
|---|---|---|
| | | Sum |
| | High | Probability |
| Sequences producing High-scoring Segment Pairs: | Score | P(N) |
| patp: AAY07036 Breast cancer associated | 1393 | 3.0e−142 |
| antigen precursor | | |
| patp: AAY05368 Human HCMV inducible | 1393 | 3.0e−142 |
| gene protein | | |
| patp: AAG64817 Human G3PDH fragment - | 1377 | 1.5e−140 |
| Homo sapiens, 327 aa. | | |
| patp: AAE04373 Mouse cancer associated | 1315 | 5.6e−134 |
| antigen OY-MC-2 | | |
| patp: AAR12995 GAP-DH - Aspergillus oryzae | 984 | 6.6e−99 |
| (ATCC 42149) | | |

In a search of sequence databases, it was found, for example, that the nucleic acid sequence of this invention has 1066 of 1225 bases (87%) identical to a gb:GENBANK-ID:AF261085|acc:AF261085.1 mRNA from Homo sapiens (glyceraldehyde-3-phosphate dehydrogenase (GADPH) mRNA). The full amino acid sequence of the protein of the invention was found to have 279 of 327 amino acid residues (85%) identical to, and 293 of 327 amino acid residues (89%) similar to, the 335 amino acid residue ptnr:TREMBLNEW-ACC:AAG01996 protein from Homo sapiens (Human) (CLONE CDABP0047 MRNA SEQUENCE). NOV89 also has homology to the other proteins shown in the BLASTP data in Table 89D.

TABLE 89D
NOV89 BLASTP results

| Gene Index/ | | Length | Identity | Positive | |
|---|---|---|---|---|---|
| Identifier | Protein/Organism | (aa) | (%) | (%) | Expect |
| gi\|7669492\|ref\| | glyceraldehyde-3- | 335 | 279/331 | 293/331 | e−153 |
| NP_002037.2\| | phosphate | | (84) | (88) | |
| (NM_002046) | dehydrogenase | | | | |
| | [Homo sapiens] | | | | |
| gi\|31645\|emb\|CAA25833.1\| | glyceraldehyde-3- | 335 | 278/331 | 293/331 | e−152 |
| (X01677) | phosphate | | (83) | (87) | |
| | dehydrogenase | | | | |
| | [Homo sapiens] | | | | |
| gi\|2407184\|gb\|AAB94053.1\| | glyceraldehyde 3- | 333 | 274/328 | 289/328 | e−149 |
| (AF017079) | phosphate | | (83) | (87) | |
| | dehydrogenase | | | | |
| | [Sus scrofa] | | | | |
| gi\|6983849\|dbj\| | glyceraldehyde-3- | 333 | 268/328 | 283/328 | e−146 |
| BAA90818.1\| | phosphate | | (81) | (85) | |
| (AB038241) | dehydrogenase | | | | |
| | [Felis catus] | | | | |
| gi\|2506441\|sp\|P00355\| | GLYCERALDEHYDE | 333 | 267/328 | 283/328 | e−146 |
| G3P_PIG | 3-PHOSPHATE | | (81) | (85) | |
| | DEHYDROGENASE | | | | |
| | (GAPDH) | | | | |

This BLASTP data is displayed graphically in the ClustalW in Table 89E. A multiple sequence alignment is given, with the NOV89 protein being shown on line 1 in a ClustalW analysis comparing the protein of the invention with the related protein sequences shown in Table 89D.

[image]

[image]

Table 89F lists the domain description from DOMAIN analysis results against NOV89. This indicates that the NOV89 sequence has properties similar to those of other proteins known to contain this domain.

TABLE 89F
Domain Analysis of NOV89
gnl|Pfam|pfam02800, gpdh_C, Glyceraldehyde 3-phosphate dehydrogenase, C-
terminal domain. GAPDH is a tetrameric NAD-binding enzyme involved in
glycolysis and glyconeogenesis. C-terminal domain is a mixed alpha/
antiparallel beta fold. SEQ ID NO: 881
CD-Length = 163 residues, 100.0% aligned
Score = 209 bits (531), Expect = 3e−55

| | | | |
|---|---|---|---|
| NOV89: | 156 | TTSCLTPLAKVIHDNFGTVEGLMTI----AATQKTMDGSYGKLWGDGHGALQNILSASTG | 211 |
| | | TT+CL PLAKV++DNFG +GLMT A QK +DG + K G A NI+ STG | |
| Sbjct: | 1 | TTNCLAPLAKVLNDNFGIEKGLMTTVHAYTADQKLVDGPHHKDLRRGRAAAPNIIPTSTG | 60 |
| NOV89: | 212 | AAKAVRKVIPELNGKLTGMAFRVPTANMSVVDLTCRLEKPTKYDDTKKVVKQASEDP-LK | 270 |
| | | AAKAV V+PELNGKLTGMAFRVPT N+SVVDLT LEKP ++ +K+A+E P LK | |
| Sbjct: | 61 | AAKAVGLVLPELNGKLTGMAFRVPTPNVSVVDLTVELEKPVTVEEINAALKEAAEGPALK | 120 |
| NOV89: | 271 | GILGYSEHQVVSSNFNSTDTHSSTFDAGAGIALNDHFVKLISWY 314 | |
| | | GILGY+E +VSS+F D HSS FDA A I LND+FVKL++WY | |
| Sbjct: | 121 | GILGYTEDPLVSSDFIG-DPHSSIFDAKATIVLNDNFVKLVAWY 163 | |

NAD-dependent glycerol-3-phosphate dehydrogenase (EC 1.1.1.8) (GPD) catalyzes the reversible reduction of dihydroxyacetone phosphate to glycerol-3-phosphate. It is a cytoplasmic protein that is active as a homodimer, each monomer containing an N-terminal NAD binding site. In insects, it acts in conjunction with a mitochondrial alpha-glycerophosphate oxidase in the alpha-glycerophosphate cycle, which is essential for the production of energy used in insect flight.

Glyceraldehyde-3-phosphate dehydrogenase (EC 1.2.2.12) (GAPDH) mRNA levels, protein, and enzymatic activity increase in 3T3-F442A adipocytes after exposure to physiological concentrations of insulin (Alexander, M., Curtis, G., Avruch, J., and Goodman, H. (1985) J. Biol. Chem. 260, 11978-11985). In order to understand the mechanism of this regulation, researchers isolated and sequenced 5.4 kilobase pairs of a 12-kilobase pair human genomic clone encoding a functional GAPDH gene. The gene consists of 9 exons and 8 introns with eukaryotic signals necessary for the transcription and translation of GAPDH mRNA. The exon sequence confirms previously published cDNA sequences for human GAPDH in muscle, liver, and erythrocytes. The organization of the human and the unique chicken GAPDH genes is strikingly similar. Although chicken exons VIII-XI have been fused into human exon 8, introns which separate exons encoding the NAD binding, catalytic, and helical domains of the GAPDH protein have been retained. Stable transfection of rodent cells with the intact human GAPDH gene resulted in the expression of a correctly initiated human GAPDH mRNA and an enzymatically active human GAPDH polypeptide. Thus, the gene contains a functional promoter and intact coding sequences. Although many processed GAPDH pseudogenes and GAPDH-like sequences are present in the human genome, Southern blot analysis of human genomic DNA using a probe derived from the 3′-untranslated region of the GAPDH gene detected only two genes, a 10-copy processed pseudogene and a single copy of the isolated gene. In contrast, a probe derived from an intron segment of the isolated gene detected only a single copy of the GAPDH gene. Collectively, these findings strongly suggest that the human genome encodes a single functional GAPDH gene.

Hopkinson et al. (1974) presented evidence that glycerol-3-phosphate dehydrogenase (EC 1.1.1.8) is a dimer of dissimilar subunits. Electrophoretic variants at each of two loci, designated GPD1 and GPD2, were described. By the method of somatic cell hybridization, Kielty and Povey (1982) assigned the presumed structural gene for alpha-glycerophosphate dehydrogenase to chromosome 12. Since this is a liver-specific enzyme, a rat hepatoma cell line was used as one of the ‘parents’ in the hybridization.

NOV89 is predicted to be expressed in at least the following tissues: liver. This information was derived by determining the tissue sources of the sequences that were included in the invention including but not limited to SeqCalling sources, public EST sources, literature sources, and/or RACE sources. Further expression data for NOV89 is provided in Example 2.

The NOV89 nucleic acids and proteins are useful in potential therapeutic applications implicated in various pathological disorders described further herein, for example, Von Hippel-Lindau (VHL) syndrome, cirrhosis, transplantation as well as other diseases, disorders and conditions. NOV89 nucleic acids encoding the Glycerol-3-Phosphate Dehydrogenase-like protein of the invention, or fragments thereof, may further be useful in diagnostic applications, wherein the presence or amount of the nucleic acid or the protein are to be assessed.

The novel nucleic acid of the invention encoding a Glycerol-3-Phosphate Dehydrogenase-like protein includes the nucleic acid whose sequence is provided in Table 89A, or a fragment thereof. The invention also includes a mutant or variant nucleic acid any of whose bases may be changed from the corresponding base shown in Table 89A while still encoding a protein that maintains its Glycerol-3-Phosphate Dehydrogenase-like activities and physiological functions, or a fragment of such a nucleic acid. The invention further includes nucleic acids whose sequences are complementary to the sequence of Table 89A, including nucleic acid fragments that are complementary to any of the nucleic acids just described. The invention additionally includes nucleic acids or nucleic acid fragments, or complements thereto, whose structures include chemical modifications. Such modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 13% of the bases may be so changed.

The novel protein of the invention includes the Glycerol-3-Phosphate Dehydrogenase-like protein whose sequence is provided in Table 89B. The invention also includes a mutant or variant protein any of whose residues may be changed from the corresponding residue shown in Table 89B while still encoding a protein that maintains its Glycerol-3-Phosphate Dehydrogenase-like activities and physiological functions, or a functional fragment thereof. In the mutant or variant protein, up to about 15% of the amino acid residues may be so changed.

These materials are further useful in the generation of antibodies that bind immunospecifically to the substances of the invention for use in therapeutic or diagnostic methods. These antibodies may be generated according to methods known in the art, using prediction from hydrophobicity charts, as described in the “Anti-NOVX Antibodies” section below.

NOV90

The disclosed NOV90 (alternatively referred to herein as CG56888-01) includes the 1686 nucleotide sequence (SEQ ID NO:289) shown in Table 90A. A NOV90 ORF begins with a Kozak consensus ATG initiation codon at nucleotides 58-60 and ends with a stop codon at nucleotides 1630-1632. The disclosed NOV90 maps to human chromosome 3.

TABLE 90A
NOV90 Nucleotide Sequence

(SEQ ID NO:289)

GTGCCTCACTGGGGTGGGGAACTTGCCTCACCTGGGGCCATTTCATATTTCTGAATCATGTGTGATAACA
GAGAACTGGAAGACAAGCCTCCAGCACCTCCCGTGCGAATGAGCAGGACCATCTTTAGCACTGGGGGCAA
AGACCCTTTGTCAGCCAATCACAGTTTGAAACCTTTGCCTTCTGTTCCAGAGGAGAAAAAGCCCAGGCAT
AAAATCATCTCCATATTCTCAGGCACAGAGAAAGGAAGTAAAAAGAAAGAAAAGGAACGGCCAGAAATTT
CTCCTCCATCTGATTTTGAACACACCATCCATGTTGGCTTTGATGCTGTTACTGGAGAATTCACTGGCAT
GCCAGAACAGTGGGCTCGATTACTACAGACCTCCAATATCACCAAACTACAGCAAAAGAAGAATCCTCAG
GCTGTGCTGGATGTCTACGACTCCAACACAGTGAAGCAGAAGTATCTGAGTTTTACTCCTCCTGAGAAAG
ATGGCTTCCCTTCTGGAACACCAGCACTGAATGCCGAGGGAACAGAAGCACCTGCAGTAGTGACAGAGGA
GGAGGACGATGATGAAGAGACTGCCCCTCCCATTATTGCCCCACCACCGGATCATATGAAATCAATTTAC
ACACGGTCTGTAATTGACCCTGTTCCTGCACCAGTTGGTGATTCAAATGTTGATGGTGGTGCCAAGTCTT
TAGACAAACAGAAAAAGAAGACTAAGATGACAGATGAAGAGATTATGGAGAAACTAAGAACTATTGTGAG
CATAGGTGACCCTAAGAAAAAAAGAAAAAAATATACAAGATATGAAAAAATTGGACAAGGGGCTTCTGGT
ACAGTTTTCACTGCTACTGACGTTGCACTGGGACAGAAGGTTGCTATCAAACAAATTAATTTACAGAAAC
AGCCAAAGAAGGAATTGATCATTAATGAGATTCTGGTAATGAAAGAATTAAAAAATCCCAACATAGTTAA
CTTCTTGGACAGTTACCTGGTAGGAGATGAATTGTTTGTGGTCGTGGAATACCTTGCTAGGGGGTCACTC
ACTGATGTGGTAACAGAAACCTGCATGGATGAAGCACAGATTGCCGCTGTATGCAGAGAGAGTTTACAGG
CATTGGAGTTTTTACATGCTAATCAAGTGATCCACAGAGACATCAAAAGTGACAGTGTACTTTTGGGAAT
GGAAGGATCGGTTAAGCTCACTGACTTTGGTTTCTGTGCCCAGATCACCCCTGAGCAGAGCAAACGCAGT
ACCGTGGTCAGAACGCCATACTGGATGGCACCAGAAGTGGTTACACGGAAGGCTTATGGCCCTAAAGTCA
ATGTATGGTCTCTGGGTATCATGGCTACTGAGATGGTAGAAGGAGAGCCTCCATACCTCAATGAAAATCC
CTTGAGGGCCTTGTGCCTAATAGCAACTAATGGAATCCCAGAACTTCAGAATCCAGAGACACTTTCCCCA
ATATTTCGGGATTTCTTAAATCGATGTTTGGAAACAGATGTGGAAAAAAGGGGTTCAGCCAAAGAATTAT
TACAGCATCTTTTCCTGAAACTAGCCAAACTGTTATCTAGCTTGACACCACTGATCATGGCAGCTAAAGA
AGCAATGAAGAGTAACCGTTAACATCACTGCTGTGGCCTCATATTCTTTTTTCCATTTTCTACAAGAAGC
CTTTTA

A NOV90 polypeptide (SEQ ID NO:290) encoded by SEQ ID NO:289 is 524 amino acids in length and is presented using the one-letter amino acid code in Table 90B. The Psort profile for NOV90 predicts that this sequence is likely to be localized to the nucleus with a certainty of 0.7000.

TABLE 90B
NOV90 Polypeptide Sequence

| | |
|---|---|
| MCDNRELEDKPPAPPVRMSRTIFSTGGKDPLSANHSLKPLPSVPEEKKPRHKIISIFSGT | (SEQ ID NO:290) |
| EKGSKKKEKERPEISPPSDFEHTIHVGFDAVTGEFTGMPEQWARLLQTSNITKLQQKKNP | |
| QAVLDVYDSNTVKQKYLSFTPPEKDGFPSGTPALNAEGTEAPAVVTEEEDDDEETAPPII | |
| APPPDHMKSIYTRSVIDPVPAPVGDSNVDGGAKSLDKQKKKTKMTDEEIMEKLRTIVSIG | |
| DPKKKRKKYTRYEKIGQGASGTVFTATDVALGQKVAIKQINLQKQPKKELIINEILVMKE | |
| LKNPNIVNFLDSYLVGDELFVVVEYLARGSLTDVVTETCMDEAQIAAVCRESLQALEFLH | |
| ANQVIHRDIKSDSVLLGMEGSVKLTDFGFCAQITPEQSKRSTVVRTPYWMAPEVVTRKAY | |
| GPKVNVWSLGIMATEMVEGEPPYLNENPLRALCLIATNGIPELQNPETLSPIFRDFLNRC | |
| LETDVEKRGSAKELLQHLFLKLAKLLSSLTPLIMAAKEAMKSNR | |

A BLAST analysis of NOV90 was run against the proprietary PatP GENESEQ Protein Patent database. It was found, for example, that the amino acid sequence of NOV90 had high homology to other proteins as shown in Table 90C.

TABLE 90C
BLASTX results from PatP database for NOV90

| | | Smallest |
|---|---|---|
| | | Sum |
| | High | Probability |
| Sequences producing High-scoring Segment Pairs: | Score | P(N) |
| patp: AAB03969 p-21 activated protein kinase | 2494 | 6.5e−259 |
| (PAK2) | | |
| patp: AAW95521 Protease activated protein | 2459 | 3.3e−255 |
| kinase I (PAK I) | | |
| patp: AAR96296 Human p21-protein activated | 2361 | 8.0e−245 |
| serine kinase | | |
| patp: AAW13379 Human p21 activated serine | 2361 | 8.0e−245 |
| kinase p65 protein | | |
| patp: AAW47119 Human p21-activated | 2361 | 8.0e−245 |
| serine kinase p65 | | |

In a search of public sequence databases, it was found, for example, that the amino acid sequence of the NOV90 protein of the present invention was found to have 503 of 524 amino acid residues (95%) similar to, the 524 amino acid residue ptnr:SWISSNEW-ACC:Q13177 protein from Homo sapiens (Human) (SERINE/THREONINE-PROTEIN KINASE PAK 2 (EC 2.7.1.-) (P21-ACTIVATED KINASE 2) (PAK-2) (PAK65) (GAMMA-PAK) (S6/H4 KINASE)). NOV90 also has homology to the other proteins shown in the BLASTP data in Table 90D.

TABLE 90D
NOV90 BLASTP results

| Gene Index/ | | Length | Identity | Positive | |
|---|---|---|---|---|---|
| Identifier | Protein/Organism | (aa) | (%) | (%) | Expect |
| gi\|3041712\|sp\| | SERINE/THREONINE-PROTEIN | 524 | 493/527 | 503/527 | 0.0 |
| Q13177\|PAK2_{—} | KINASE PAK 2 (P21- | | (93) | (94) | |
| Human | ACTIVATED KINASE 2) (PAK- | | | | |
| | 2) (PAK65) (GAMMA-PAK) | | | | |
| | (S6/H4 KINASE) | | | | |
| gi\|4505599\|ref\| | p21 (CDKN1A)-activated | 525 | 493/528 | 503/528 | 0.0 |
| NP_002568.1\| | kinase 2; novel serine | | (93) | (94) | |
| (NM_002577) | kinase; hPAK65 | | | | |
| | [Homo sapiens] | | | | |
| gi\|16758002\|ref\| | p21 (CDKN1A)-activated | 524 | 483/527 | 498/527 | 0.0 |
| NP_445758.1\| | kinase 2 | | (91) | (93) | |
| (NM_053306) | [Rattus norvegicus] | | | | |
| gi\|2499647\|sp\| | SERINE/THREONINE-PROTEIN | 524 | 484/527 | 498/527 | 0.0 |
| Q29502\|PAK2_{—} | KINASE PAK 2 (P21- | | (91) | (93) | |
| RABIT | ACTIVATED KINASE 2) | | | | |
| | (PAK-2) (GAMMA-PAK) | | | | |
| | (P21-ACTIVATED PROTEIN | | | | |
| | KINASE I) (PAKI) | | | | |
| gi\|6288680\|gb\| | PAK2 [Rattus norvegicus] | 524 | 481/527 | 498/527 | 0.0 |
| AAF06695.1\|U19967_1 | | | (91) | (94) | |
| (U19967) | | | | | |

This BLASTP data is displayed graphically in the ClustalW in Table 90E. A multiple sequence alignment is given, with the NOV90 protein being shown on line 1 in a ClustalW analysis comparing the protein of the invention with the related protein sequences shown in Table 90D.

[image]

[image]

Table 90F lists the domain description from DOMAIN analysis results against NOV90. This indicates that the NOV90 sequence has properties similar to those of other proteins known to contain this domain.

TABLE 90F
Domain Analysis of NOV90

gnl|Smart|smart00220, S_TKc, Serine/Threonine protein kinases, catalytic
domain; Phosphotransferases. Serine or threonine-specific kinase subfamily.
SEQ ID NO:882
CD-Length = 256 residues, 94.9% aligned
Score = 251 bits (640), Expect = 1e−67

| | | | |
|---|---|---|---|
| NOV90: | 255 | IGQGASGTVFTATDVALGQKVAIKQINLQKQPKK--ELIINEILVMKELKNPNIVNFLDS | 312 |
| | | +G+GA G V+ A D G+ VAIK I +K KK E I+ EI ++K+L +PNIV D | |
| Sbjct: | 7 | LGKGAFGKVYLARDKKTGKLVAIKVIKKEKLKKKKRERILREIKILKKLDHPNIVKLYDV | 66 |
| NOV90: | 313 | YLVGDELFVVVEYLARGSLTDVVTET-CMDEAQIAAVCRESLQALEFLHANQVIHRDIKS | 371 |
| | | + D+L++V+EY G L D++ + + E + R+ L ALE+LH+ +IHRD+K | |
| Sbjct: | 67 | FEDDDKLYLVMEYCEGGDLFDLLKKRGRLSEDEARFYARQILSALEYLHSQGIIHRDLKP | 126 |
| NOV90: | 372 | DSVLLGMEGSVKLTDFGFCAQITPEQSKRSTVVRTPYWMAPEVVTRKAYGPKVNVWSLGI | 431 |
| | | +++LL +G VKL DFG Q+ + +T V TP +MAPEV+ K YG V++WSLG+ | |
| Sbjct: | 127 | ENILLDSDGHVKLADFGLAKQLDSGGTLLTTFVGTPEYMAPEVLLGKGYGKAVDIWSLGV | 186 |
| NOV90: | 432 | MATEMVEGEPPYLNENPLRALCLIATNGIPELQNPE-TLSPIFRDFLNRCLETDVEKRGS | 490 |
| | | + E++ G+PP+ ++ L AL P PE +SP +D + + L D EKR + | |
| Sbjct: | 187 | ILYELLTGKPPFPGDDQLLALFKKIGKPPPPFPPPEWKISPEAKDLIKKLLVKDPEKRLT | 246 |
| NOV90: | 491 | AKE 493 | |
| | | A+E | |
| Sbjct: | 247 | AEE 249 | |

Serine/threonine kinases are an extensive family of enzymes that catalyzes the phosphorylation of serine or threonine residues on its target protein. Protein kinases share a conserved catalytic core common to both serine/threonine and tyrosine protein kinases. This domain contains residues, which are specific to the distinct types of protein kinases

The S6/H4 kinase purified from human placenta catalyzes phosphorylation of the S6 ribosomal protein, histone H4, and myelin basic protein. In vitro activation of the p60 S6/H4 kinase requires removal of an autoinhibitory domain by mild trypsin digestion and autophosphorylation of the catalytic domain (p40 S6/H4 kinase). The two autophosphorylation/autoactivation sites contain the sequences SSMVGTPY (site 1) and SVIDPVPAPVGDSHVDGAAK (site 2). These sequences identify S6H4 kinase as the rac-activated PAK65 (Martin, G. A., Bollag, G., McCormick, F. and Abo, A. (1995) EMBO J. 14, 1971-1978). Site 1 phosphorylation is most rapid, but activation does not occur until site 2 is autophosphorylated. The site 1 phosphorylation occurs by an intramolecular mechanism whereas site 2 autophosphorylation occurs by an intermolecular mechanism. A model is proposed in which phosphorylation of sites 1 and 2 occurs sequentially. The model proposes that trypsin treatment of the inactive holoenzyme removes an inhibitory rac-binding domain which blocks MgATP access to the catalytic site. The pseudosubstrate domain at site 1 is autophosphorylated and subsequent bimolecular autophosphorylation at site 2 fully opens the catalytic site. Phosphorylation by a regulatory protein kinase may occur at site 2 in vivo.

NOV90 is predicted to be expressed in at least the following tissues: brain, cerebellum, skeletal muscle, ovary, thymus and spleen. This information was derived by determining the tissue sources of the sequences that were included in the invention including but not limited to SeqCalling sources, public EST sources, literature sources, and/or RACE sources. Further expression data for NOV90 is provided in Example 2.

The NOV90 nucleic acids and proteins are useful in potential therapeutic applications implicated in various pathological disorders described further herein, for example, Von Hippel-Lindau (VHL) syndrome, Alzheimer's disease, stroke, tuberous sclerosis, hypercalceimia, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, neuroprotection, hemophilia, hypercoagulation, idiopathic thrombocytopenic purpura, immunodeficiencies, hemophilia, hypercoagulation, immunodeficiencies, graft vesus host disease as well as other diseases, disorders and conditions. NOV90 nucleic acids encoding the PAK2-like protein of the invention, or fragments thereof, may further be useful in diagnostic applications, wherein the presence or amount of the nucleic acid or the protein are to be assessed.

The novel nucleic acid of the invention encoding a serine/threonine-protein kinase PAK 2-like protein includes the nucleic acid whose sequence is provided in Table 90A, or a fragment thereof. The invention also includes a mutant or variant nucleic acid any of whose bases may be changed from the corresponding base shown in Table 90A while still encoding a protein that maintains its serine/threonine-protein kinase PAK 2-like activities and physiological functions, or a fragment of such a nucleic acid. The invention further includes nucleic acids whose sequences are complementary to the sequence of Table 90A, including nucleic acid fragments that are complementary to any of the nucleic acids just described.

The invention additionally includes nucleic acids or nucleic acid fragments, or complements thereto, whose structures include chemical modifications. Such modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

The novel protein of the invention includes the serine/threonine-protein kinase PAK 2-like protein whose sequence is provided in Table 90B. The invention also includes a mutant or variant protein any of whose residues may be changed from the corresponding residue shown in Table 90B while still encoding a protein that maintains its serine/threonine-protein kinase PAK 2-like activities and physiological functions, or a functional fragment thereof. In the mutant or variant protein, up to about 6% of the amino acid residues may be so changed.

These materials are further useful in the generation of antibodies that bind immunospecifically to the substances of the invention for use in therapeutic or diagnostic methods. These antibodies may be generated according to methods known in the art, using prediction from hydrophobicity charts, as described in the “Anti-NOVX Antibodies” section below.

NOV91

The disclosed NOV91 (alternatively referred to herein as CG56779-01) includes the 404 nucleotide sequence (SEQ ID NO:291) shown in Table 91A. A NOV91 ORF begins with a Kozak consensus ATG initiation codon at nucleotides 25-27 and ends with a stop codon at nucleotides 373-375. The disclosed NOV91n maps to human chromosome 3.

TABLE 91A
NOV91 Nucleotide Sequence

(SEQ ID NO:291)

AATGGGTCTCTTTTTCTACAGCCCATGCCCTTCCTAGAGCTAGACACGAACTTGCCTGCCAACCAAGTGC
CTGCAGGGTTGGAGAAATGGCTCTGCGCCACAGCCTCCATCCTGGGCAAACCCAAGGATCATGTGAACAT
GATGGGTGTAGCGGGCCTGACCATGGTGCTGAGTAGGTCCACTGAGCCCTGGGCGCAGCTGTTCATCTCC
TCCACCAGCATGATGGACACCACTGAGGAGAACCGCAGCCACAGCACCCACTTCTTCGAGTTCCTCACCG
AGGAGCTGGCCCTGGGCCAGGACCAGATAATTTTCCACTTTTCCCCCCTGGAGCCCTGGCAGACTGGCAA
GAAGGGGATGGTCATAACTTTTTAGTGACTGGCCTCGAGGGATCCAGGGCATCT

A NOV91 polypeptide (SEQ ID NO:292) encoded by SEQ ID NO:291 is 116 amino acids in length and is presented using the one-letter amino acid code in Table 91B. The Psort profile for NOV91 predicts that this sequence has no signal peptide and is likely to be localized to microbodies with a certainty of 0.6400. In alternative embodiments, a NOV91 polypeptide is located to the cytoplasm with a certainty of 0.4500.

TABLE 91B
NOV91 Polypeptide Sequence

| | |
|---|---|
| MPFLELDTNLPANQVPAGLEKWLCATASILGKPKDHVNMMGVAGLTMVLSRSTEPWAQLF | (SEQ ID NO:292) |
| ISSTSMMDTTEENRSHSTHFFEFLTEELALGQDQIIFHFSPLEPWQTGKKGMVITF | |

A BLAST analysis of NOV91 was run against the proprietary PatP GENESEQ Protein Patent database. It was found, for example, that the amino acid sequence of NOV91 had high homology to other proteins as shown in Table 91C.

TABLE 91C
BLASTX results from PatP database for NOV91

| | | Smallest |
|---|---|---|
| | | Sum |
| | High | Probability |
| Sequences producing High-scoring Segment Pairs: | Score | P(N) |
| patp: AAR83048 Human macrophage migration | 399 | 6.5e−37 |
| inhibitory factor | | |
| patp: AAY44997 Human D-dopachrome | 369 | 9.8e−34 |
| tautomerase (DDT) | | |
| patp: AAB43733 Human cancer associated | 262 | 2.1e−22 |
| protein sequence | | |
| patp: AAM22110 Peptide #8544 encoded by probe | 183 | 5.0e−14 |
| patp: AAM38563 Peptide #12600 encoded by probe | 183 | 5.0e−14 |

In a search of sequence databases, it was found, for example, that the nucleic acid sequence of this invention has 283 of 289 bases (97%) identical to a gb:GENBANK-ID:AP000500|acc:AP000500.1 mRNA from Homo sapiens (genomic DNA, chromosome 3p21.3, clone:603 to 320, anti-oncogene region, section 3/3). The full amino acid sequence of the protein of the invention was found to have 82 of 117 amino acid residues (70%) identical to, and 91 of 117 amino acid residues (77%) similar to, the 118 amino acid residue ptnr:pir-id:JE0162 protein from human (dopachrome Delta-isomerase (EC 5.3.3.12)). NOV91 also has homology to the other proteins shown in the BLASTP data in Table 91D.

TABLE 91D
NOV91 BLASTP results

| Gene Index/ | | Length | Identity | Positive | |
|---|---|---|---|---|---|
| Identifier | Protein/Organism | (aa) | (%) | (%) | Expect |
| gi\|4503291\|ref\| | D-dopachrome | 118 | 82/117 | 91/117 | 1e−38 |
| NP_001346.1\| | tautomerase | | (70) | (77) | |
| (NM_001355) | [Homo sapiens] | | | | |
| gi\|4699610\| | Chain A, | 117 | 81/116 | 90/116 | 5e−38 |
| pdb\|1DPT\|A | D-Dopachrome | | (69) | (76) | |
| | Tautomerase | | | | |
| gi\|7512375\|pir\|\| | D-dopachrome | 118 | 80/117 | 89/117 | 7e−38 |
| G02438 | tautomerase - | | (68) | (75) | |
| | human | | | | |
| gi\|13162287\| | D-dopachrome | 118 | 71/117 | 94/117 | 6e−34 |
| ref\|NP_{—} | tautomerase | | (60) | (79) | |
| 077045.1\| | [Rattus | | | | |
| (NM_024131) | norvegicus] | | | | |
| gi\|6753618\|ref\| | D-dopachrome | 118 | 67/117 | 92/117 | 2e−32 |
| NP_034157.1\| | tautomerase | | (57) | (78) | |
| (NM_010027) | [Mus musculus] | | | | |

This BLASTP data is displayed graphically in the ClustalW in Table 91E. A multiple sequence alignment is given, with the NOV91 protein being shown on line 1 in a ClustalW analysis comparing the protein of the invention with the related protein sequences shown in Table 91D.

[image]

[image]

Table 91F lists the domain description from DOMAIN analysis results against NOV91. This indicates that the NOV91 sequence has properties similar to those of other proteins known to contain this domain.

TABLE 91F
Domain Analysis of NOV91

gnl|Pfam|pfam01187, MIF, Macrophage migration inhibitory factor (MIF).
SEQ ID NO: 883
CD-Length = 114 residues, 100.0% aligned
Score = 126 bits (316), Expect = 8e−31

| | | | |
|---|---|---|---|
| NOV91: | 2 | PFLELDTNLPANQVPAGLEKWLCA-TASILGKPKDHVNMMGVAGLTMVLSRSTEPWAQLF | 60 |
| | | P +DTNLPAN VPAG EK L A A LGKP+D + + G MV ST+P A | |
| Sbjct: | 1 | PMFTIDTNLPANSVPAGFEKRLTAALAKALGKPEDRIAVHIRPGQAMVFGGSTDPCAVCS | 60 |
| NOV91: | 61 | ISSTSMMDTTEENRSHSTHFFEFLTEELALGQDQIIFHFSPLEPWQTGKKGMVIT 115 | |
| | | I S ++ E+NRSHS F+FL +EL L +D++ F LE Q G G + | |
| Sbjct: | 61 | IKSIGVV-GAEQNRSHSALLFKFLAKELGLPKDRVYIRFFDLEAAQVGFNGTTMA 114 | |

D-Dopachrome tautomerase (DDT) shares a low homologous amino acid sequence (33% homology) with the macrophage migration inhibitory factor (MIF) yet possesses similar tautomerase activity. MIF is a cytokine involved in inflammatory reactions and immune responses. While MIF is a secreted protein, it is not processed from a larger precursor. Whereas recent studies have identified MIF as a pituitary hormone and immunoregulator, less is known about the structural basis of these physiological functions and the real significance of tautomerase activity. D-dopachrome tautomerase, which is related to MIF, is a mammalian cytoplasmic enzyme involved in melanin biosynthesis that tautomerizes 2-carboxy-2,3-dihydroindole-5, 6-quinone (D-dopachrome) with concomitant decarboxylation to give 5,6-dihydroxyindole (DHI). It is a protein of 117 residues, and acts as a homotrimer.

NOV91 is predicted to be expressed in at least the following tissues: largely in the liver, and to lesser extent in other organs, including the heart, lung, pancreas; and placenta. This information was derived by determining the tissue sources of the sequences that were included in the invention including but not limited to SeqCalling sources, public EST sources, literature sources, and/or RACE sources. Further expression data for NOV91 is provided in Example 2.

The NOV91 nucleic acids and proteins are useful in potential therapeutic applications implicated in various pathological disorders described further herein, for example, Von Hippel-Lindau (VHL) syndrome, Alzheimer's disease, stroke, tuberous sclerosis, hypercalceimia, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, neuroprotection, hemophilia, hypercoagulation, idiopathic thrombocytopenic purpura, immunodeficiencies, hemophilia, hypercoagulation, immunodeficiencies, graft vesus host disease as well as other diseases, disorders and conditions. NOV91 nucleic acids encoding the D-Dopachrome tautomerase-like protein of the invention, or fragments thereof, may further be useful in diagnostic applications, wherein the presence or amount of the nucleic acid or the protein are to be assessed.

The novel nucleic acid of the invention encoding a D-dopachrome tautomerase-like protein includes the nucleic acid whose sequence is provided in Table 91A, or a fragment thereof. The invention also includes a mutant or variant nucleic acid any of whose bases may be changed from the corresponding base shown in Table 91A while still encoding a protein that maintains its D-dopachrome tautomerase-like activities and physiological functions, or a fragment of such a nucleic acid. The invention further includes nucleic acids whose sequences are complementary to those just described, including nucleic acid fragments that are complementary to any of the nucleic acids just described.

The invention additionally includes nucleic acids or nucleic acid fragments, or complements thereto, whose structures include chemical modifications. Such modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 3% of the residues may be so changed.

The novel protein of the invention includes the D-dopachrome tautomerase-like protein whose sequence is provided in Table 91B. The invention also includes a mutant or variant protein any of whose residues may be changed from the corresponding residue shown in Table 91B while still encoding a protein that maintains its D-dopachrome tautomerase-like activities and physiological functions, or a functional fragment thereof. In the mutant or variant protein, up to about 30% of the bases may be so changed.

These materials are further useful in the generation of antibodies that bind immunospecifically to the substances of the invention for use in therapeutic or diagnostic methods. These antibodies may be generated according to methods known in the art, using prediction from hydrophobicity charts, as described in the “Anti-NOVX Antibodies” section below.

NOV92

The disclosed NOV92 (alternatively referred to herein as CG56904-01) includes the 1311 nucleotide sequence (SEQ ID NO:293) shown in Table 92A. A NOV92 ORF begins with a Kozak consensus ATG initiation codon at nucleotides 19-21 and ends with a stop codon at nucleotides 1282-1284.

TABLE 92A
NOV92 Nucleotide Sequence

(SEQ ID NO:293)

GGAGCTCCCACACTTTCAATGGGGAGGCCCACCCAGTGGCCGAGCCTGCTGCTGCTCCTGCTGTTGCCGG
GGCCCCCGCCCGTCGCCGGCTTGGAAGACGCTGCCTTCCCCCACCTGGGGGAGAGCTTGCAGCCCCTGCC
CCGGGCCTGTCCCCTGCGCTGCTCCTGCCCCCGAGTCGACACTGTGGACTGTGATGGCTTGGACCTTCGA
GTGTTCCCGGACAACATCACCAGAGCCGCTCAGCACCTCTCCCTGCAGAACAACCAGCTCCAGGAACTCC
CCTACAATGAGCTGTCCCGCCTCAGTGGCCTGCGAACCCTCAACCTCCACAACAACCTCATCTCCTCCGA
AGGCCTGCCTGACGAGGCCTTCGAGTCCCTCACCCAGCTGCAGCACCTCTGCGTGGCTCACAACAAGAAC
AATCTCATCTCCAAGGTGCCCCGAGGAGCCCTGAGCCGCCAGACTCAACTCCGTGAGCTCTACCTCCAGC
ACAACCAGCTGACAGACAGTGGCCTGGATGCCACCACCTTCAGCAAGCTGCATAGCCTTGAATACCTGGA
TCTCTCCCACAACCAGCTGACCACAGTGCCCGCCGGCCTGCCCCGGACCCTGGCTATCCTGCACCTGGGC
CGCAACCGCATCCGGCAGGTGGAGGCGGCTCGGCTGCACGGGGCGCGTGGTCTGCGCTATTTGTTGCTGC
AGCACAACCAGCTGGGGAGCTCAGGGCTGCCCGCCGGGGCTCTGCGGCCGCTGCGGGGCCTGCACACGCT
GCACCTCGATGGCAATGGGCTGGACCGCGTGCCTCCAGCCCTGCCCCGCCGCCTGCGTGCCCTGGTGCTG
CCCCACAACCACGTGGCCGCGCTGGGTGCCCGTGACCTGGTCGCCACACCGGGCCTGACGGAGCTTAACC
TGGCCTATAACCGCCTGGCCAGCGCCCGTGTGCACCACCGGGCCTTCCGCCGGTTGCGTGCCCTGCGCAG
CCTCGACCTGGCAGGGAATCAGCTAACCCGGCTGCCCATGGGCCTGCCCACTGGCCTGCGCACCCTGCAG
CTGCAACGCAACCAGCTGCGGATGCTCGAGCCCGAGCCTCTGGCCGGCCTGGACCAACTGCGGGAGCTCA
GCCTGGCGCACAACCGGCTCCGGGTCGGCGACATCGGGCCAGGCACCTGGCATGAGCTCCAAGCCCTCCA
GGTCAGGCACAGGCTGGTTAGCCACACTGTCCCCAGGGCCCCTCCATCCCCCTGCCTGCCCTGCCACGTC
CCAAACATTCTAGTTAGCTGGTAAAGCAATCAGAACAAGAAAATGATAAGA

A NOV92 polypeptide (SEQ ID NO:294) encoded by SEQ ID NO:293 is 421 amino acids in length and is presented using the one-letter amino acid code in Table 92B. The Psort profile for NOV92 predicts that this sequence has a signal peptide and is likely to be secreted with a certainty of 0.4419. The Signal P predicts a likely cleavage site for a NOV92 peptide is between positions 24 and 25, i.e., at the dash in the sequence VAG-LE.

TABLE 92B
NOV92 Polypeptide Sequence

| | |
|---|---|
| MGRPTQWPSLLLLLLLPGPPPVAGLEDAAFPHLGESLQPLPRACPLRCSCPRVDTVDCDGL | (SEQ ID NO:294) |
| DLRVFPDNITRAAQHLSLQNNQLQELPYNELSRLSGLRTLNLHNNLISSEGLPDEAFESLT | |
| QLQHLCVAHNKNNLISKVPRGALSRQTQLRELYLQHNQLTDSGLDATTFSKLHSLEYLDLS | |
| HNQLTTVPAGLPRTLAILHLGRNRIRQVEAARLHGARGLRYLLLQHNQLGSSGLPAGALRP | |
| LRGLHTLHLDGNGLDRVPPALPRRLRALVLPHNHVAALGARDLVATPGLTELNLAYNRLAS | |
| ARVHHRAFRRLRALRSLDLAGNQLTRLPMGLPTGLRTLQLQRNQLRMLEPEPLAGLDQLRE | |
| LSLAHNRLRVGDIGPGTWHELQALQVRHRLVSHTVPRAPPSPCLPCHVPNILVSW | |

A BLAST analysis of NOV92 was run against the proprietary PatP GENESEQ Protein Patent database. It was found, for example, that the amino acid sequence of NOV92 had high homology to other proteins as shown in Table 92C.

TABLE 92C
BLASTX results from PatP database for NOV92

| | | Smallest |
|---|---|---|
| | | Sum |
| | High | Probability |
| Sequences producing High-scoring Segment Pairs: | Score | P(N) |
| patp: AAY13396 Amino acid sequence of | 690 | 4.9e−70 |
| protein PRO332 | | |
| patp: AAB33425 Human PRO332 protein | 690 | 4.9e−70 |
| patp: AAB80264 Human PRO332 protein - | 690 | 4.9e−70 |
| Homo sapiens, 642 aa. | | |
| patp: AAU12356 Human PRO332 polypeptide | 690 | 4.9e−70 |
| sequence | | |
| patp: AAM41258 Human polypeptide SEQ | 334 | 5.0e−30 |
| ID NO 6189 | | |

In a search of sequence databases, it was found, for example, that the nucleic acid sequence of this invention has 1310 of 1312 bases (99%) identical to a gb:GENBANK-ID:AK027100|acc:AK027100.1 mRNA from Homo sapiens (cDNA: FLJ23447 fis, clone HSI03346). The full amino acid sequence of the protein of the invention was found to have 290 of 291 amino acid residues (99%) identical to, and 290 of 291 amino acid residues (99%) similar to, the 363 amino acid residue ptnr:TREMBLNEW-ACC:BAB15657 protein from Homo sapiens (Human) (CDNA: FLJ23447 FIS, CLONE HSI03346). NOV92 also has homology to the other proteins shown in the BLASTP data in Table 92D.

TABLE 92D
NOV92 BLASTP results

| Gene Index/ | | Length | Identity | Positive | |
|---|---|---|---|---|---|
| Identifier | Protein/Organism | (aa) | (%) | (%) | Expect |
| gi\|13376224\|ref\| | hypothetical protein | 363 | 290/291 | 290/291 | e−136 |
| NP_079101.1\| | FLJ23447 [Homo sapiens] | | (99) | (99) | |
| (NM_024825) | | | | | |
| gi\|11761721\|gb\| | decorin | 359 | 87/281 | 137/281 | 4e−27 |
| AAG40157.1\| | [Oreochromis niloticus] | | (30) | (47) | |
| AF247822_1 | | | | | |
| (AF247822) | | | | | |
| gi\|6759315\|dbj\| | biglycan [Xenopus | 368 | 90/289 | 134/289 | 1e−26 |
| BAA90246.1\| | laevis] | | (31) | (46) | |
| (AB037269) | | | | | |
| >gi\|8134605\|sp\| | BONE PROTEOGLYCAN II | 360 | 94/298 | 144/298 | 4e−25 |
| Q9XSD9\|PGS2_{—} | PRECURSOR (PG-S2) | | (31) | (47) | |
| PIG | (DECORIN) | | | | |
| gi\|129949\|sp\| | BONE PROTEOGLYCAN II | 360 | 88/292 | 140/292 | 6e−25 |
| P21793\|PGS2_BOVIN | PRECURSOR (PG-S2) | | (30) | (47) | |

This BLASTP data is displayed graphically in the ClustalW in Table 92E. A multiple sequence alignment is given, with the NOV92 protein being shown on line 1 in a ClustalW analysis comparing the protein of the invention with the related protein sequences shown in Table 92D.

[image]

[image]

Leucine-rich repeats (LRRs) are relatively short motifs (22-28 residues in length) found in a variety of cytoplasmic, membrane and extracellular proteins. Although these proteins are associated with widely different functions, a common property involves protein-protein interaction. Little is known about the 3D structure of LRRs, although it is believed that they can form amphipathic structures with hydrophobic surfaces capable of interacting with membranes. In vitro studies of a synthetic LRR from Drosophila Toll protein have indicated that the peptides form gels by adopting beta-sheet structures that form extended filaments. These results are consistent with the idea that LRRs mediate protein-protein interactions and cellular adhesion. Other functions of LRR-containing proteins include, for example, binding to enzymes and vascular repair. The 3-D structure of ribonuclease inhibitor, a protein containing 15 LRRs, has been determined, revealing LRRs to be a new class of alpha/beta fold. LRRs form elongated non-globular structures and are often flanked by cysteine rich domains.

NOV92 is predicted to be expressed in at least the following tissues: colon, brain. This information was derived by determining the tissue sources of the sequences that were included in the invention including but not limited to SeqCalling sources, public EST sources, literature sources, and/or RACE sources. Further expression data for NOV92 is provided in Example 2.

The NOV92 nucleic acids and proteins are useful in potential therapeutic applications implicated in various pathological disorders described further herein, for example, Von Hippel-Lindau (VHL) syndrome, Alzheimer's disease, stroke, tuberous sclerosis, hypercalceimia, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, neuroprotection, hemophilia, hypercoagulation, idiopathic thrombocytopenic purpura, immunodeficiencies, hemophilia, hypercoagulation, immunodeficiencies, graft vesus host disease as well as other diseases, disorders and conditions. NOV92 nucleic acids encoding the LRR-like protein of the invention, or fragments thereof, may further be useful in diagnostic applications, wherein the presence or amount of the nucleic acid or the protein are to be assessed.

The novel nucleic acid of the invention encoding a secreted leucine-rich repeat (LRR) protein-like protein includes the nucleic acid whose sequence is provided in Table 92A, or a fragment thereof. The invention also includes a mutant or variant nucleic acid any of whose bases may be changed from the corresponding base shown in Table 92A while still encoding a protein that maintains its secreted leucine-rich repeat (LRR) protein-like activities and physiological functions, or a fragment of such a nucleic acid. The invention further includes nucleic acids whose sequences are complementary to the sequence of Table 92A, including nucleic acid fragments that are complementary to any of the nucleic acids just described.

The invention additionally includes nucleic acids or nucleic acid fragments, or complements thereto, whose structures include chemical modifications. Such modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 1% of the bases may be so changed.

The novel protein of the invention includes the secreted leucine-rich repeat (LRR) protein-like protein whose sequence is provided in Table 92B. The invention also includes a mutant or variant protein any of whose residues may be changed from the corresponding residue shown in Table 92B while still encoding a protein that maintains its Secreted leucine-rich repeat (LRR) protein-like activities and physiological functions, or a functional fragment thereof. In the mutant or variant protein, up to about 1% of the amino acid residues may be so changed.

These materials are further useful in the generation of antibodies that bind immunospecifically to the substances of the invention for use in therapeutic or diagnostic methods. These antibodies may be generated according to methods known in the art, using prediction from hydrophobicity charts, as described in the “Anti-NOVX Antibodies” section below.

NOV93

The disclosed NOV93 (alternatively referred to herein as CG56277-01) includes the 1518 nucleotide sequence (SEQ ID NO:295) shown in Table 93A. A NOV93 ORF begins with a Kozak consensus ATG initiation codon at nucleotides 2-4 and ends with a stop codon at nucleotides 1556-1558. The disclosed NOV93 maps to human chromosome 2.

TABLE 93A
NOV93 Nucleotide Sequence

(SEQ ID NO:295)

CATGAGGGGACTACCTATCAGCAGCAGCACCGGCTACGTGGTCGAGGACGGGTTCACTGCGACCGTGCAG
CAGCTCTTCGCCAGTGACCAGGGACTCACCTACAACGACTTCTTGATTCTCCCAGGATTCATAGACTTCA
TAGCTGATGAGGTGGACCTGACCTCAGCCCTGACCCAACCGGTCACTCTGAAGACGCCGCTGATCTCCTC
CCCCATGGACACTGTGACAGAGGCCGACCTGGCCATCGTGATGGCTCTGATGGGAGGTACTGGTTTCATT
CACCACAACTGCACCCCAGAGTTCCAGGCCAGTGAGGTGCAGAAGGTCAAGAAGTTTGAACCGGGCTTTA
TCACACACCCCGTGGTGCTGAGCCCCTTGCACACTGTGGGTGATGTGTTGGAGGCCAAGATGCGTCATGG
CTTCTCTGGCATCCCCATCACTGAGACGGGTACCATGGGCAGCAAGCTGGTGGGCATCGTCACCTCCCGA
GACATCGACTTTCTTGCTGAGAAGGACCACACCACCCTCCTCAGTGAGGTGATGATGCCAAGGATCAAGC
TAGTGGTGGCTCCAGCAAGCAGTGTGAGGTTGAAAGAGGCAAATGAGATCCTGCAGCTTAGTAAGAAAGG
AAAGCTGCCTATCGTCAATGATCGCGATGAGCTGGTGGCCATTATCACCTGCACCGCGCTGAAGAACCGA
GACTACCCTGTGGCCTCCAAGGATTCCCATGAGCAGCTGCTGGGCGGGGCAGCTGTGGGTACCCATGAGG
ATGACAAATACCACCTGGACCTGCTCACCCAGGTAGGCGTCAATGTCATAGGCTTGGACTCGTCCCAAGG
GAACTCGGTGTATCAGATCGCCATGGTGCATTACATCAAACAAAAGTACCCCCACCTCCAGGTGATTGGG
GGGAACGTGGTGACAGCAGCCCAGGCCAACAACCTGATTGACGCTGGTGTGGATGGGCTGGGCAGGGGCA
TGGACTGCGCGGCTGGCTCCATCTACATCAACCAGGAAGTGATAGCCTGCAGTCAGCCCCAGGGCACTGC
TGTGTACAAGGTGGCCAAGCATACCCAGAACTTTGGTGTGCCCATCATAGCCGATGGTGGCATCCAGACC
ATGGGGCATGTGGTCAAGGCCCTGGCCCTAGGAGCCTCCACAGTGATGATGGGCTCCCTGCTGGCCGCCA
CCATGGAGGCCCCCGGCGAGTGCTTCTTCTCAGACGGAATGCAGCTCAAGAAGTACCAGGGCATGGGCTC
ACTGGATGCCATGGAGAAGAGCAGCAGCAGCCAGAAACAATACTTCAACGACGGGGATAAGGCGAAGATC
ACGCAGGATGTCTTGGGCTCCATCCAGGACAAAGGGTCCATTCAGAAGTTCGTGCCCTACCTCATAGTGG
GCATCCAGCATGGCTGCCAGGATATCGGGGCCCACAGCCTGTCTGTCCTTCGGTCCATGATGTACTCAGG
GGAGCTCAAGTTTGAGAAGCAGACCATGTCAGCCCAGATCGACGGTGGCATCCATGGCCTGCACTCTTAC
GAGAAGTGGCTGTACTGAGGACAGCGGTGCAGGGCGAGATG

A NOV93 polypeptide (SEQ ID NO:296) encoded by SEQ ID NO:295 is 518 amino acids in length and is presented using the one-letter amino acid code in Table 93B. The Psort profile for NOV93 predicts that this sequence is likely to be localized to the cytoplasm with a certainty of 0.4500. In alternative embodiments, a NOV93 polypeptide is located to lysosomes with a certainty of 0.1921, or, to microbodies with a certainty of 0.3346.

TABLE 93B
NOV93 Polypeptide Sequence

| | |
|---|---|
| MRGLPISSSTGYVVEDGFTATVQQLFASDQGLTYNDFLILPGFIDFIADEVDLTSALTQP | (SEQ ID NO:296) |
| VTLKTPLISSPMDTVTEADLAIVMALMGGTGFIHHNCTPEFQASEVQKVKKFEPGFITHP | |
| VVLSPLHTVGDVLEAKMRHGFSGIPITETGTMGSKLVGIVTSRDIDFLAEKDHTTLLSEV | |
| MMPRIKLVVAPASSVRLKEANEILQLSKKGKLPIVNDRDELVAIITCTALKNRDYPVASK | |
| DSHEQLLGGAAVGTHEDDKYHLDLLTQVGVNVIGLDSSQGNSVYQIAMVHYIKQKYPHLQ | |
| VIGGNVVTAAQANNLIDAGVDGLGRGMDCAAGSIYINQEVIACSQPQGTAVYKVAKHTQN | |
| FGVPIIADGGIQTMGHVVKALALGASTVMMGSLLAATMEAPGECFFSDGMQLKKYQGMGS | |
| LDAMEKSSSSQKQYFNDGDKAKITQDVLGSIQDKGSIQKFVPYLIVGIQHGCQDIGAHSL | |
| SVLRSMMYSGELKFEKQTMSAQIDGGIHGLHSYEKWLY | |

A BLAST analysis of NOV93 was run against the proprietary PatP GENESEQ Protein Patent database. It was found, for example, that the amino acid sequence of NOV93 had high homology to other proteins as shown in Table 93C.

TABLE 93C
BLASTX results from PatP database for NOV93

| | | Smallest |
|---|---|---|
| | | Sum |
| | High | Probability |
| Sequences producing High-scoring Segment Pairs: | Score | P(N) |
| patp: AAR05431 Chinese hamster | 1874 | 3.2e−193 |
| IMPDH - Cricetulus sp, 514 aa. | | |
| patp: AAR05432 Human IMPDH - | 1872 | 5.3e−193 |
| Homo sapiens, 514 aa. | | |
| patp: AAY08965 A. gossypii | 980 | 1.8e−98 |
| inosine-monophosphate dehydrogenase | | |
| patp: AAG30888 Arabidopsis thaliana | 973 | 9.7e−98 |
| protein fragment | | |
| patp: AAG43108 Arabidopsis thaliana | 949 | 3.4e−95 |
| protein fragment | | |

In a search of sequence databases, it was found, for example, that the nucleic acid sequence of this invention has 1402 of 1567 bases (89%) identical to a gb:GENBANK-ID:HUMIMPH|acc:J05272.1 mRNA from Homo sapiens (Human IMP dehydrogenase type 1 mRNA). The full amino acid sequence of the protein of the invention was found to have 438 of 513 amino acid residues (85%) identical to, and 462 of 513 amino acid residues (90%) similar to, the 514 amino acid residue ptnr:SWISSNEW-ACC:P20839 protein from Homo sapiens (Human) (INOSINE-5′-MONOPHOSPHATE DEHYDROGENASE 1 (EC 1.1.1.205) (IMP DEHYDROGENASE 1) (IMPDH-I) (IMPD 1)). NOV93 also has homology to the other proteins shown in the BLASTP data in Table 93D.

TABLE 93D
NOV93 BLASTP results

| Gene Index/ | | Length | Identity | Positive | |
|---|---|---|---|---|---|
| Identifier | Protein/Organism | (aa) | (%) | (%) | Expect |
| gi\|17453301\|ref\| | IMP (inosine | 514 | 440/514 | 464/514 | 0.0 |
| XP_004627.5\| | monophosphate) | | (85) | (89) | |
| (XM_004627) | dehydrogenase 1 | | | | |
| | [Homo sapiens] | | | | |
| gi\|124417\|sp\| | INOSINE-5′- | 514 | 438/514 | 462/514 | 0.0 |
| P20839\|IMD1_{—} | MONOPHOSPHATE | | (85) | (89) | |
| HUMAN | DEHYDROGENASE 1 | | | | |
| | (IMP DEHYDROGENASE 1) | | | | |
| | (IMPDH-I) (IMPD 1) | | | | |
| gi\|4504687\|ref\| | IMP (inosine | 514 | 434/514 | 458/514 | 0.0 |
| NP_000874.1\| | monophosphate) | | (84) | (88) | |
| (NM_000883) | dehydrogenase 1; | | | | |
| | sWSS2608 | | | | |
| | [Homo sapiens] | | | | |
| gi\|6754344\|ref\| | inosine 5′- | 514 | 430/514 | 462/514 | 0.0 |
| NP_035959.1 | phosphate | | (83) | (89) | |
| (NM_011829) | dehydrogenase 1 | | | | |
| | [Mus musculus] | | | | |
| gi\|16549223\|dbj\| | unnamed protein | 489 | 418/514 | 440/514 | 0.0 |
| BAB70780.1\| | product Homo | | (81) | (85) | |
| (AK054640) | sapiens | | | | |

This BLASTP data is displayed graphically in the ClustalW in Table 93E. A multiple sequence alignment is given, with the NOV93 protein being shown on line 1 in a ClustalW analysis comparing the protein of the invention with the related protein sequences shown in Table 93D.

[image]

[image]

[image]

Table 93F lists the domain description from DOMAIN analysis results against NOV93. This indicates that the NOV93 sequence has properties similar to those of other proteins known to contain this domain.

TABLE 93F
Domain Analysis of NOV93

gnl|Pfam|pfam00478, IMPDH_C, IMP dehydrogenase/GMP reductase C terminus. This
family is involved in biosynthesis of guanosine nucleotide biosynthesis.
Members of this family contain a TIM barrel structure. The alignment does not
contain the whole TIM barrel domain. The alignment is truncated after the
insert domain (2 CBS domains pfam00571) found in the inosine-5′-monophosphate
dehydrogenase structure. This family should always be associated with
pfam01574. This family is a member of the common phosphate binding site TIM
barrel family. SEQ ID NO:884
CD-Length=222 residues, 100.0% aligned
Score=261 bits (666), Expect=9e−71

| | | | |
|---|---|---|---|
| NOV93: | 264 | LLTQVGVNVIGLDSSQGNSVYQIAMVHYIKQKYPHLQVIGGNVVTAAQANNLIDAGVDGL | 323 |
| | | L + GV+VI LDSS G S QI + +I++KYP +QVI GNVVT A LIDAG D + | |
| Sbjct: | 1 | ALVEAGVDVICLDSSNGYSEVQIDFIRWIREKYPTVQVIAGNVVTGEMAEELIDAGADAV | 60 |
| NOV93: | 324 | GRGMDCAAGSIYINQEVIACSQPQGTAVYKVAKHTQNFGVPIIADGGIQTMGHVVKALAL | 383 |
| | | G+ GSI I +EV +PQ TAV +VA + +P+I+DGGI GH+ KALA | |
| Sbjct: | 61 | KVGI--GPGSICITREVAGIGRPQATAVLEVADASHGLNIPVISDGGITNPGHMAKALAG | 118 |
| NOV93: | 384 | GASTVMMGSLLAATMEAPGECFFSDGMQLKKYQGMGSLDAMEKSSSSQKQYFNDGDKAKI | 443 |
| | | GA VM+GSLLA T EAPGE F DG + K Y+GMGSL AM+K S +YF K + | |
| Sbjct: | 119 | GADFVMIGSLLAGTEEAPGEVVFKDGKKYKLYRGMGSLTAMKKYQGSVARYFASKQKLSV | 178 |
| NOV93: | 444 | TQDVLGSIQDKGSIQKFVPYLIVGIQHGCQDIGAHSLSVLRSMM 487 | |
| | | + V G + KG + + V L+ G++ C IGA L LR | |
| Sbjct: | 179 | EEGVTGYVPYKGDVSRTVHDLLGGLRSSCTYIGATKLKQLRKRA 222 | |

Inosine-5-prime-monophosphate dehydrogenase (EC 1.1.1.205) catalyzes the formation of xanthine monophosphate (XMP) from IMP. In the purine de novo synthetic pathway, IMP dehydrogenase is positioned at the branch point in the synthesis of adenine and guanine nucleotides and is thus the rate-limiting enzyme in the de novo synthesis of guanine nucleotides. Inhibition of cellular IMP dehydrogenase activity results in an abrupt cessation of DNA synthesis and a cell-cycle block at the G1-S interface. Collart and Huberman (1988) used a polyclonal antibody directed against the purified protein to isolate human and Chinese hamster IMP dehydrogenase cDNA clones. The sequence of these clones demonstrated an open reading frame for a protein containing 514 amino acids. The molecular mass of the produced protein was 56 kD, which is the observed molecular mass of the purified protein and of the immunoprecipitated in vitro translation product.

A high order of conservation of the IMP dehydrogenase protein was indicated by the finding that human and Chinese hamster cDNA clones differed by only 8 amino acids. Natsumeda et al. (1990) isolated two distinct cDNAs (types I and II) encoding IMP dehydrogenase from a human spleen cDNA library. Both clones encode proteins of 514 residues showing 84% sequence identity. Type I mRNA was found to be the main species in normal leukocytes, and type II (146691) predominated in human ovarian tumors. Using PCR primers specific for type II IMPDH, Glesne et al. (1993) screened a panel of human/Chinese hamster cell somatic hybrids and a separate deletion panel of chromosome 3 hybrids and localized the gene to 3p24.2-p21.2.

The gene was also localized on a map of two overlapping YACs and found to span no more than 12.5 kb of genomic DNA. From cloning and sequencing IMPD2, Glesne and Huberman (1994) determined that the gene spans approximately 5 kb and is interrupted by 12 introns. The transcriptional start sites were determined by S1 nuclease mapping to be somewhat heterogeneous but the predominant mRNA species showed a 5-prime end at 102 and 85 nucleotides from the translational initiation codon. Zimmermann et al. (1995) also cloned the human gene and noted that it has 14 exons spanning approximately 5.8 kb. They also characterized regulatory elements in the 5-prime flanking region of the gene.

NOV93 is predicted to be expressed in at least the following tissues: brain, prosencephalon/forebrain, diencephalon, pituitary gland. This information was derived by determining the tissue sources of the sequences that were included in the invention including but not limited to SeqCalling sources, public EST sources, literature sources, and/or RACE sources. Further expression data for NOV93 is provided in Example 2.

The NOV93 nucleic acids and proteins are useful in potential therapeutic applications implicated in various pathological disorders described further herein, for example, Von Hippel-Lindau (VHL) syndrome, Alzheimer's disease, stroke, tuberous sclerosis, hypercalceimia, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, neuroprotection, hemophilia, hypercoagulation, idiopathic thrombocytopenic purpura, immunodeficiencies, hemophilia, hypercoagulation, immunodeficiencies, graft vesus host disease as well as other diseases, disorders and conditions. NOV93 nucleic acids encoding the inosine-5-prime-monophosphate dehydrogenase-like protein of the invention, or fragments thereof, may further be useful in diagnostic applications, wherein the presence or amount of the nucleic acid or the protein are to be assessed.

The novel nucleic acid of the invention encoding a inosine-5′-monophosphate dehydrogenase-like protein includes the nucleic acid whose sequence is provided in Table 93A, or a fragment thereof. The invention also includes a mutant or variant nucleic acid any of whose bases may be changed from the corresponding base shown in Table 93A while still encoding a protein that maintains its inosine-5′-monophosphate dehydrogenase-like activities and physiological functions, or a fragment of such a nucleic acid. The invention further includes nucleic acids whose sequences are complementary to the sequence of Table 93A, including nucleic acid fragments that are complementary to any of the nucleic acids just described.

The invention additionally includes nucleic acids or nucleic acid fragments, or complements thereto, whose structures include chemical modifications. Such modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 11% of the bases may be so changed.

The novel protein of the invention includes the inosine-5′-monophosphate dehydrogenase-like protein whose sequence is provided in Table 93B. The invention also includes a mutant or variant protein any of whose residues may be changed from the corresponding residue shown in Table 93B while still encoding a protein that maintains its inosine-5′-monophosphate dehydrogenase-like activities and physiological functions, or a functional fragment thereof. In the mutant or variant protein, up to about 15% of the amino acid residues may be so changed.

These materials are further useful in the generation of antibodies that bind immunospecifically to the substances of the invention for use in therapeutic or diagnostic methods. These antibodies may be generated according to methods known in the art, using prediction from hydrophobicity charts, as described in the “Anti-NOVX Antibodies” section below.

NOV94

The disclosed NOV94 (alternatively referred to herein as CG56281-01) includes the 1573 nucleotide sequence (SEQ ID NO:297) shown in Table 94A. A NOV94 ORF begins with a Kozak consensus ATG initiation codon at nucleotides 1-3 and ends with a stop codon at nucleotides 1564-1566. The disclosed NOV94 maps to human chromosome 3.

TABLE 94A
NOV94 Nucleotide Sequence

(SEQ ID NO:297)

ATGGCGCGGAAGCAGGACCCGAAGCCTAAATTCCAGGAGGGTGAGCGAGTGCTGTGCTTTCATGGGCCTC
TTCTTTATGAAGCAAAGTGTGTAAAGGTTGCCATAAAGGACAAACAAGTGAAATACTTCATACATTACAG
TGGTTGGAATAAAAATTGGGATGAGTGGGTTCCGGAGAGCAGAGTACTCAAATACGTGGACACCAATGAA
AATCGTAGATTGGCCAGGGAAATTCGTAGATTACAGCATAAATTGGCAAGAAATGCTGTAGCTCACCTGA
GGAGCAAGAGAGAAAGAAGCAGCCGCTCCAGGTTGCTTGGTGCTGACTCTGTCTTAAAAGGCCTCTCCAT
CGAAGAAAAAAATGAAAATGATGAAAACTCATTAAGCAGTTCCTCTGACAGTAGTGAAGACAAGGATGAA
AAAATAAGTGAAGAATGTGATATTGAAGAAAAGACTGAAGTGAAAGAAGAACCGGAGCTTCAAACAAAAA
GGGAAATGGAAGAAAGAACAGTAACTCTAGAAATCCCTGAAGTTCTGAAGAGGCAGCTGGAGGATGATTG
TTACTACATTAATCGGAGGAAACGGTTAGTGCAACTTCCATGCCACACCAACATCATAACGATTTTGGAA
TCCTATGTGAAGCATTTTGCTATCAGTGCAGCCTTTTCAGCCAATGAGAGGCCTCGTCACCATCACGCTA
TGCCACATGCCAGCATGAACGTGCCTTATATCCCAGCAGAAAAGAATATTGACCTTTGTAAGGAGATGGT
GGATGGATTAAGAATAACCTTTGATTACACTCTCCCGTTGGTTTTACTCTATCCCTATGAACAAGCTCAG
TATAAAAAGGTGACTGCATCTAAGGTTTTTCTTGCAATTAAGGAAAGTGCCACAAATACTAATAGGAGCC
AGGAGAAGCTCTCTCCCAGCTTACGTTTGTTGAATCCATCCAGGCCGCAGTCTACAGAGAGTCAGTCGAC
CAGCGGTGAACCAGCCACCCCTAAAAGGCGCAAAGCCGAGCCGCAAGCAGTGCAGTCTCTGAGGCGGTCC
TCGCCCCACACCGCCAACTGTGACAGGCTTTCTAAGAGCAGCACCTCACCTCAGCCCAAGCGCTGGCAGC
AGGACATGTCCACCAGTGTGCCCAAGCTGTTCCTGCACCTGGAAAAGAAGACACCTGTGCATAGCAGATC
ATCTTCACCTACTCTGACTCCTAGCCAGGAAGGGAGTCCTGTGTTTGCTGGCTTTGAAGGGAGAAGAACT
AATGAAATAAATGAGGTCCTCTCCTGGAAGCTCGTACCTGACAATTACCCACCAGGTGACCAGCCACCTC
CACCCTCTTACATTTACGGGGCGCAACATTTGCTGCGATTGTTTGTCAAACTTCCAGAAATTCTTGGAAA
AATGTCCTTTACTGAGAAGAATCTGAAGGCTTTATTGAAGCACTTTGATCTCTTTGTGAGGTTTTTAGCA
GAATACCACGATGACTTCTTCCCAGAGTCAGCTTACGTCGCTGCCTCTGAGGTGCATTACAGCACCAGGA
ACCCCCAGGCAGTCAATAAGTGTTGATGGTTCT

A NOV94 polypeptide (SEQ ID NO:298) encoded by SEQ ID NO:297 is 521 amino acids in length and is presented using the one-letter amino acid code in Table 94B. The Psort profile for NOV94 predicts that this sequence is likely to be localized to the nucleus with a certainty of 0.9700. In alternative embodiments, a NOV94 polypeptide is localized to microbodies with a certainty of 0.3000.

TABLE 94B
NOV94 Polypeptide Sequence

| | |
|---|---|
| MARKQDPKPKFQEGERVLCFHGPLLYEAKCVKVAIKDKQVKYFIHYSGWNKNWDEWVPES | (SEQ ID NO:298) |
| RVLKYVDTNENRRLAREIRRLQHKLARNAVAHLRSKRERSSRSRLLGADSVLKGLSIEEK | |
| NENDENSLSSSSDSSEDKDEKISEECDIEEKTEVKEEPELQTKREMEERTVTLEIPEVLK | |
| RQLEDDCYYINRRKRLVQLPCHTNIITILESYVKHFAISAAFSANERPRHHHAMPHASMN | |
| VPYIPAEKNIDLCKEMVDGLRITFDYTLPLVLLYPYEQAQYKKVTASKVFLAIKESATNT | |
| NRSQEKLSPSLRLLNPSRPQSTESQSTSGEPATPKRRKAEPQAVQSLRRSSPHTANCDRL | |
| SKSSTSPQPKRWQQDMSTSVPKLFLHLEKKTPVHSRSSSPTLTPSQEGSPVFAGFEGRRT | |
| NEINEVLSWKLVPDNYPPGDQPPPPSYIYGAQHLLRLFVKLPEILGKMSFTEKNLKALLK | |
| HEDLFVRFLAEYHDDFFPESAYVAASEVHYSTRNPQAVNKC | |

A BLAST analysis of NOV94 was run against the proprietary PatP GENESEQ Protein Patent database. It was found, for example, that the amino acid sequence of NOV94 had high homology to other proteins as shown in Table 94C.

TABLE 94C
BLASTX results from PatP database for NOV94

| | | Smallest |
|---|---|---|
| | | Sum |
| | High | Probability |
| Sequences producing High-scoring Segment Pairs: | Score | P(N) |
| patp: AAW96184 Senescence protein | 465 | 2.9e−51 |
| derived from human | | |
| patp: AAB60085 Human transport | 465 | 2.9e−51 |
| protein TPPT-5 | | |
| patp: AAU32295 Novel human | 430 | 3.4e−40 |
| secreted protein #2786 | | |
| patp: AAM64801 Human brain | 239 | 3.5e−19 |
| expressed single exon probe | | |
| patp: AAM77558 Human bone | 239 | 3.5e−19 |
| marrow expressed probe | | |

In a search of sequence databases, it was found, for example, that the nucleic acid sequence of this invention has 1381 of 1581 bases (87%) identical to a gb:GENBANK-ID:AF117065|acc:AF117065.1 mRNA from Homo sapiens (male-specific lethal-3 homolog 1 (MSL3L1) mRNA). The full amino acid sequence of the protein of the invention was found to have 414 of 520 amino acid residues (79%) identical to, and 457 of 520 amino acid residues (87%) similar to, the 521 amino acid residue ptnr:SPTREMBL-ACC:Q9Y5Z8 protein from Homo sapiens (Human) (MALE-SPECIFIC LETHAL-3 HOMOLOG 1). NOV94 also has homology to the other proteins shown in the BLASTP data in Table 94D.

TABLE 94D
NOV94 BLASTP results

| Gene Index/ | | Length | | Positive | |
|---|---|---|---|---|---|
| Identifier | Protein/Organism | (aa) | Identity (%) | (%) | Expect |
| gi\|17975757\|ref\| | male-specific lethal 3- | 521 | 413/529 | 456/529 | 0.0 |
| NP_523353.1\| | like 1 isoform a; | | (78) | (86) | |
| (NM_078629) | drosophila MSL3-like 1 | | | | |
| | [Homo sapiens] | | | | |
| gi\|17975759\|ref\| | male-specific lethal 3- | 462 | 389/468 | 420/468 | 0.0 |
| NP_523354.1\| | like 1 isoform b; | | (83) | (89) | |
| (NM_078630) | drosophila MSL3-like 1 | | | | |
| | [Homo sapiens] | | | | |
| gi\|14764458\|ref\| | male-specific lethal-3 | 496 | 387/498 | 427/498 | 0.0 |
| XP_045715.1\| | (Drosophila)-like 1 | | (77) | (85) | |
| (XM_045715) | [Homo sapiens] | | | | |
| gi\|11545735\|ref\| | male-specific lethal-3 | 466 | 346/472 | 396/472 | e−168 |
| NP_034962.2\| | homolog 1 (Drosophila) | | (73) | (83) | |
| (NM_010832) | [Mus musculus] | | | | |
| gi\|17975761\|ref\| | male-specific | 355 | 312/354 | 335/354 | e−162 |
| NP_006791.2\| | lethal 3-like 1 | | (88) | (94) | |
| (NM_006800) | isoform c; drosophila | | | | |
| | MSL3-like 1 | | | | |
| | [Homo sapiens] | | | | |

This BLASTP data is displayed graphically in the ClustalW in Table 94E. A multiple sequence alignment is given, with the NOV94 protein being shown on line 1 in a ClustalW analysis comparing the protein of the invention with the related protein sequences shown in Table 94D.

[image]

[image]

Table 94F lists the domain description from DOMAIN analysis results against NOV94. This indicates that the NOV94 sequence has properties similar to those of other proteins known to contain this domain.

TABLE 94F
Domain Analysis of NOV94

gnl|Smart|smart00298, CHROMO, Chromatin
organization modifier domain SEQ ID NO:885
CD-Length = 55 residues,
Score = 42.0 bits (97), Expect = 9e−05

| | | | |
|---|---|---|---|
| NOV94: | 36 | KDKQVKYFIHYSGWNKNWDEWVPESRVLK | 64 |
| | | K +++Y + + G++ D W PE +L | |
| Sbjct: | 14 | KKGELEYLVKWKGYSYREDTWEPEENLLN | 42 |

The Drosophila male-specific lethal (msl) genes regulate transcription from the male X chromosome in a dosage compensation pathway that equalizes X-linked gene expression in males and females. The members of this gene family, including msl1, msl2, msl3, mle, and mof, encode proteins with no sequence similarity to known proteins. However, mutations in each of these genes produce a similar phenotype: sex-specific lethality of male embryos caused by the failure of mutants to increase transcription from the single male X chromosome.

The MSL gene products assemble into a multiprotein transcriptional activation complex at hundreds of sites along the chromatin of the X chromosome. By searching sequence databases with the sequence of a BAC clone that maps to Xp22.3, Prakash et al. (1999) identified a human homolog of Drosophila msl3, MSL3-like-1 (MSL3L1). They isolated a cDNA containing a complete MSL3L1 coding sequence. The deduced 521-amino acid MSL3L1 protein shares 30% overall sequence identity with Drosophila MSL3 and 86% identity with mouse Msl3l1. Three segments of the Drosophila MSL3 protein are highly conserved in MSL3L1, including two putative chromodomains, one at the N terminus and the other at the C terminus. Chromodomains, which form a characteristic tertiary structure and can interact with components of chromatin, have been implicated to play roles in chromatin organization and transcriptional regulation. MSL3L1 also contains a putative nuclear localization signal, a putative leucine zipper motif within the second chromodomain, and two potential tyrosine kinase phosphorylation sites.

Prakash et al. (1999) identified human fetal kidney cDNAs representing an alternatively spliced MSL3L1 transcript that lacks exon 2. The predicted protein, which is referred to as isoform 2, is identical to the first isoform from amino acid 62 to the C terminus but does not contain the first 26 amino acids of the N-terminal chromodomain. Northern blot analysis detected a major 2.4-kb MSL3L1 transcript in all tissues examined, namely liver, pancreas, heart, lung, kidney, skeletal muscle, brain, and placenta, with highest expression in skeletal muscle and heart. A 2.6-kb transcript unique to skeletal muscle was also found. Northern blot analysis of E7, E11, E15, and E17 mouse embryos detected approximately equal levels of Msl3l1 expression in all embryos. The MSL3L1 gene spans 17 kb and contains 13 exons. It is transcribed from telomere to centromere. Prakash et al. (1999) showed that the MSL3L1 gene undergoes X inactivation.

NOV94 is predicted to be expressed in at least the following tissues: lung, testis, B-cell. This information was derived by determining the tissue sources of the sequences that were included in the invention including but not limited to SeqCalling sources, public EST sources, literature sources, and/or RACE sources. Further expression data for NOV94 is provided in Example 2.

The NOV94 nucleic acids and proteins are useful in potential therapeutic applications implicated in various pathological disorders described further herein, for example, Von Hippel-Lindau (VHL) syndrome, Alzheimer's disease, stroke, tuberous sclerosis, hypercalceimia, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, neuroprotection, hemophilia, hypercoagulation, idiopathic thrombocytopenic purpura, immunodeficiencies, hemophilia, hypercoagulation, immunodeficiencies, graft vesus host disease as well as other diseases, disorders and conditions. NOV94 nucleic acids encoding the MSL3L1-like protein of the invention, or fragments thereof, may further be useful in diagnostic applications, wherein the presence or amount of the nucleic acid or the protein are to be assessed.

The novel nucleic acid of the invention encoding a male-specific lethal 3-like 1-like protein includes the nucleic acid whose sequence is provided in Table 94A, or a fragment thereof. The invention also includes a mutant or variant nucleic acid any of whose bases may be changed from the corresponding base shown in Table 94A while still encoding a protein that maintains its male-specific lethal 3-like 1-like activities and physiological functions, or a fragment of such a nucleic acid. The invention further includes nucleic acids whose sequences are complementary to the sequence of Table 94A, including nucleic acid fragments that are complementary to any of the nucleic acids just described.

The invention additionally includes nucleic acids or nucleic acid fragments, or complements thereto, whose structures include chemical modifications. Such modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 13% of the bases may be so changed.

The novel protein of the invention includes the male-specific lethal 3-like 1-like protein whose sequence is provided in Table 94B. The invention also includes a mutant or variant protein any of whose residues may be changed from the corresponding residue shown in Table 94B while still encoding a protein that maintains its male-specific lethal 3-like 1-like activities and physiological functions, or a functional fragment thereof. In the mutant or variant protein, up to about 21% of the amino acid residues may be so changed.

These materials are further useful in the generation of antibodies that bind immunospecifically to the substances of the invention for use in therapeutic or diagnostic methods. These antibodies may be generated according to methods known in the art, using prediction from hydrophobicity charts, as described in the “Anti-NOVX Antibodies” section below.

NOV95

The disclosed NOV95 (alternatively referred to herein as CG56975-01) includes the 1323 nucleotide sequence (SEQ ID NO:299) shown in Table 95A. A NOV95 ORF begins with a Kozak consensus ATG initiation codon at nucleotides 35-37 and ends with a stop codon at nucleotides 1301-1303. The disclosed NOV95 maps to human chromosome 1.

TABLE 95A
NOV95 Nucleotide Sequence

(SEQ ID NO:299)

GCAATTCCTTTTCAATATTTATATATTTCAGAAAATGTCACTGAAATTCACAAATGCAAAACGGATTGAA
GGACTTGATAGTAATGTGTGGATTGAATTTACCAAATTGGCTGCAGACCCTTCTGTTGTGAATCTTGGCC
AAGGCTTTCCAGATATATCCCCTCCTACATATGTAAAAGAAGAATTATCAAAGATTGCAGCAATCGATAG
CCTGAATCAGTATACACGAGGCTTTGGCCATCCATCACTTGTGAAAGCTCTGTCCTATCTGTATGAAAAG
CTTTATCAAAAGCAAATTGATTCAAATAAAGAAATCCTTGTGACAGTAGGAGCATATGGATCTCTTTTTA
ACACCATTCAAGCATTAATTGATGAGGGACAGGTCATACTAATAGTGCCTTTCTATGACTGCTATGAGCC
CATGGTGAGAATGGCTGGAGCAACACCTGTTTTTATTCCCCTGAGATCTGTAAGTTTGGGAAAAAGATGG
TCTAGTTCTGACTGGACATTAGATCCTCAAGAACTGGAAAGTAAATTTAATTCCAAAACCAAAGCTATTA
TACTAAATACTCCACATAACCCACTTGGCAAGGTATATAACAGAGAGGAACTGCAAGTAATTGCTGACCT
TTGCATCAAATATGACACACTCTGCATCAGCGATGAGGTTTATGAATGGCTTGTATATTCTGGAAATAAG
CACTTAAAAATAGCTACTTTTCCAGGTATGTGGGAGAGAACAATAACAATAGGAAGTGCTGGAAAGACTT
TCAGTGTAACTGGCTGGAAGGTAGGCTGGTCCATTGGTCCAAATCATTTGATAAAACATTTACAGACAGT
TCAACAAAACACGATTTATACTTGTGCAACTCCTTTACAGGAAGCCTTGGCTCAAGCTTTCTGGATTGAC
ATCAAGCGCATGGATGACCCAGAATGTTACTTTAATTCTTTGCCAAAAGAGTTAGAAGTAAAAAGAGATC
GGATGGTACGTTTACTTGAAAGTGTTGGCCTAAAACCCATAGTTCCTGATGGAGGATACTTCATCATCGC
TGATGTGTCTATTTTCATTGTGGTTTTAGATCCAGACCTCTCTGATATGAAGAATAATGAGCCTTATGAC
TATAAGTTTGTGAAATGGATGACTAAACATCAGAAACTATCAGCCATCCCCGTTTCAGCATTCTGTAACT
CAGAGACTAAATCACAGTTTGAGAAGTTTGTGCGTTTTTGCTTCATTAAAGTAAGTTCCCTGCTCGATGC
TGCTGAAGAAATCATCAAGGCATGGAGTGTACAGAAGTCTTGATTTGTGCAGAATGGATTAAT

A NOV95 polypeptide (SEQ ID NO:300) encoded by SEQ ID NO:299 is 422 amino acids in length and is presented using the one-letter amino acid code in Table 95B. The Psort profile for NOV95 predicts that this sequence is a Type II membrane protein, and is likely to be localized at the plasma membrane with a certainty of 0.4400. In alternative embodiments, a NOV95 polypeptide is located to microbodies with a certainty of 0.3691.

TABLE 95B
NOV95 Polypeptide Sequence

| | |
|---|---|
| MSLKFTNAKRIEGLDSNVWIEFTKLAADPSVVNLGQGFPDISPPTYVKEELSKIAAIDSL | (SEQ ID NO:300) |
| NQYTRGFGHPSLVKALSYLYEKLYQKQIDSNKEILVTVGAYGSLFNTIQALIDEGQVILI | |
| VPFYDCYEPMVRMAGATPVFIPLRSVSLGKRWSSSDWTLDPQELESKFNSKTKAIILNTP | |
| HNPLGKVYNREELQVIADLCIKYDTLCISDEVYEWLVYSGNKHLKIATFPGMWERTITIG | |
| SAGKTFSVTGWKVGWSIGPNHLIKHLQTVQQNTIYTCATPLQEALAQAFWIDIKRMDDPE | |
| CYFNSLPKELEVKRDRMVRLLESVGLKPIVPDGGYFIIADVSIFIVVLDPDLSDMKNNEP | |
| YDYKFVKWMTKHQKLSAIPVSAFCNSETKSQFEKFVRFCFIKVSSLLDAAEEIIKAWSVQ | |
| KS | |

A BLAST analysis of NOV95 was run against the proprietary PatP GENESEQ Protein Patent database. It was found, for example, that the amino acid sequence of NOV95 had high homology to other proteins as shown in Table 95C.

TABLE 95C
BLASTX results from PatP database for NOV95

| | | Smallest |
|---|---|---|
| | | Sum |
| | High | Probability |
| Sequences producing High-scoring Segment Pairs: | Score | P(N) |
| patp: AAY54591 Amino acid sequence | 2121 | 2.2e−219 |
| of a human transferase | | |
| patp: AAR89906 Human kynurenine | 1171 | 1.0e−118 |
| aminotransferase (KAT) | | |
| patp: AAR89896 Rat kynurenine | 1130 | 2.2e−114 |
| aminotransferase (KAT) | | |
| patp: AAR89897 Rat kynurenine | 1130 | 2.2e−114 |
| aminotransferase (KAT) | | |
| patp: AAR89898 Rat kynurenine | 1130 | 2.2e−114 |
| aminotransferase (KAT) | | |

In a search of sequence databases, it was found, for example, that the nucleic acid sequence of this invention has 806 of 915 bases (88%) identical to a gb:GENBANK-ID:AF091090|acc:AF091090.1 mRNA from Homo sapiens (clone 669 unknown mRNA, complete sequence). The full amino acid sequence of the protein of the invention was found to have 229 of 415 amino acid residues (55%) identical to, and 300 of 415 amino acid residues (72%) similar to, the 419 amino acid residue ptnr:SPTREMBL-ACC:Q9W6U2 protein from Fugu rubripes (Japanese pufferfish) (Takifugu rubripes) (CYSTEINE CONJUGATE BETA-LYASE). NOV95 also has homology to the other proteins shown in the BLASTP data in Table 95D.

TABLE 95D
NOV95 BLASTP results

| Gene Index/ | | Length | | Positive | |
|---|---|---|---|---|---|
| Identifier | Protein/Organism | (aa) | Identity (%) | (%) | Expect |
| gi\|12654031\|gb\| | Similar to CG6950 gene | 290 | 280/294 | 284/294 | e−162 |
| AAH00819.1\| | product [Homo sapiens] | | (95) | (96) | |
| AAH00819 | | | | | |
| (BC000819) | | | | | |
| gi\|5002565\|emb\| | cysteine conjugate beta- | 419 | 230/418 | 301/418 | e−134 |
| CAB44334.1\| | lyase [Takifugu rubripes] | | (55) | (71) | |
| (Y17462) | | | | | |
| gi\|4757928\|ref\| | cytoplasmic cysteine | 422 | 215/421 | 299/421 | e−128 |
| NP_004050.1\| | conjugate-beta lyase; | | (51) | (70) | |
| (NM_004059) | glutamine-phenylpyruvate | | | | |
| | aminotransferase | | | | |
| | [Homo sapiens] | | | | |
| gi\|15425868\|gb\| | kynurenine | 477 | 224/420 | 284/420 | e−126 |
| AAK97625.1\| | aminotransferase [Aedes | | (53) | (67) | |
| AF395204_1 | aegypti] | | | | |
| (AF395204) | | | | | |
| gi\|7299520\|gb\| | CG6950 gene product [alt | 417 | 220/417 | 289/417 | e−125 |
| AAF54707.1\| | 3] | | (52) | (68) | |
| (AE003693) | [Drosophila melanogaster] | | | | |

This BLASTP data is displayed graphically in the ClustalW in Table 95E. A multiple sequence alignment is given, with the NOV95 protein being shown on line 1 in a ClustalW analysis comparing the protein of the invention with the related protein sequences shown in Table 95D.

[image]

[image]

Table 95F lists the domain description from DOMAIN analysis results against NOV95. This indicates that the NOV95 sequence has properties similar to those of other proteins known to contain this domain.

TABLE 95F
Domain Analysis of NOV95

gnl|Pfam|pfam00155, aminotran_1_2, Aminotransferase class I and II
SEQ ID NO:886
CD-Length = 316 residues, 94.3% aligned
Score = 117 bits (292), Expect = 2e−27

| | | | |
|---|---|---|---|
| NOV95: | 93 | EILVTVGAYGSLFNTIQALIDEGQVILI-VPFYDCYEPMVRMAGATPVFIPLRSVSLGKR | 151 |
| | | ++L GA I G +L+ P Y Y ++ AG + L V L | |
| Sbjct: | 17 | QVLAGTGAKEVAALFISCFAAPGDAVLVPDPTYPIYSDVLNHAGGI---VRLYPVPLRSS | 73 |
| NOV95: | 152 | WSSSDWTLDPQELESKFNSKTKAIILNTPHNPLGKVYNREELQVIADLCIKYDTLCISDE | 211 |
| | | + D+ + LE +K +++ PHNP G +L+ + DL +++ L + DE | |
| Sbjct: | 74 | NHN-DFKALEEALEEA-PEGSKVVLVANPHNPTGMDGTLADLEKLLDLAKEHNILLLVDE | 131 |
| NOV95: | 212 | VYEWLVYSGNKHLKIATFPGMWERTITIGSAGKTFSVTGWKVGWSIG---------PNHL | 262 |
| | | Y V+ G IA ++ + + S K F + G ++G + G + + | |
| Sbjct: | 132 | AYAGGVFGGLDGASIAELLDEYDNLLVVQSLSKNFGLAGKRLGGAAGGIVAGSAASFDRV | 191 |
| NOV95: | 263 | IKHLQTVQQNTIYTCATPLQEALAQAFWIDIKRMDDPECYFNSLPKELEVKRDRMVRLLE | 322 |
| | | + + AT A + + D E + L + | |
| Sbjct: | 192 | SSQSRALLF------ATSSAPPAVGAAIVALILQD------------KERLERWLKELKK | 233 |
| NOV95: | 323 | SVGLKPIVPDGGYFIIADVSIFIVVLDPDLSDMKNNEPYDYKFVKWMTKHQKLSAIPVSA | 382 |
| | | +GL+ ++ G+ + D S I+ L + + +P S | |
| Sbjct: | 234 | MLGLRVLLSRAGFVLWLDPS-GILPLWTFEDQA--------GLFSALLLEEHGVVVPGSE | 284 |
| NOV95: | 383 | FCNSETKSQFEKFVRFCFIKVSSL-LDAAEEIIKA 416 | |
| | | F R ++ LD E I+A | |
| Sbjct: | 285 | FPTVPPGW-----GRISLAGLTDETLDELLEAIRA 314 | |

Aminotransferases share certain mechanistic features with other pyridoxal-phosphate dependent enzymes, such as the covalent binding of the pyridoxal-phosphate group to a lysine residue. On the basis of sequence similarity, these various enzymes can be grouped into subfamilies, one of which is the class I. Class I aminotransferases include cysteine conjugate beta-lyase. Living organisms employ a variety of metabolic pathways when detoxifying xenobiotic compounds, including the formation of cysteine S-conjugates via glutathione conjugation. Kidney cysteine conjugate beta-lyase (glutamine transaminase K, kyneurenine aminotransferase, EC 2.6.1.64) metabolises the cysteine conjugates of certain halogenated alkenes and alkanes to form reactive metabolites which can produce nephrotoxicity and neurotoxicity in experimental animals and man.

NOV95 is predicted to be expressed in at least the following tissues: kidney, liver, colon, gall bladder. This information was derived by determining the tissue sources of the sequences that were included in the invention including but not limited to SeqCalling sources, public EST sources, literature sources, and/or RACE sources. Further expression data for NOV95 is provided in Example 2.

The NOV95 nucleic acids and proteins are useful in potential therapeutic applications implicated in various pathological disorders described further herein, for example, Von Hippel-Lindau (VHL) syndrome, Alzheimer's disease, stroke, tuberous sclerosis, hypercalceimia, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, neuroprotection, hemophilia, hypercoagulation, idiopathic thrombocytopenic purpura, immunodeficiencies, hemophilia, hypercoagulation, immunodeficiencies, graft vesus host disease as well as other diseases, disorders and conditions. NOV95 nucleic acids encoding the aminotransferase-like protein of the invention, or fragments thereof, may further be useful in diagnostic applications, wherein the presence or amount of the nucleic acid or the protein are to be assessed.

The novel nucleic acid of the invention encoding a cysteine conjugate beta-lyase-like protein includes the nucleic acid whose sequence is provided in Table 95A, or a fragment thereof. The invention also includes a mutant or variant nucleic acid any of whose bases may be changed from the corresponding base shown in Table 95A while still encoding a protein that maintains its cysteine conjugate beta-lyase-like activities and physiological functions, or a fragment of such a nucleic acid. The invention further includes nucleic acids whose sequences are complementary to the sequence of Table 95A, including nucleic acid fragments that are complementary to any of the nucleic acids just described.

The invention additionally includes nucleic acids or nucleic acid fragments, or complements thereto, whose structures include chemical modifications. Such modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 12% of the bases may be so changed.

The novel protein of the invention includes the cysteine conjugate beta-lyase-like protein whose sequence is provided in Table 95B. The invention also includes a mutant or variant protein any of whose residues may be changed from the corresponding residue shown in Table 95B while still encoding a protein that maintains its cysteine conjugate beta-lyase-like activities and physiological functions, or a functional fragment thereof. In the mutant or variant protein, up to about 45% of the amino acid residues may be so changed.

These materials are further useful in the generation of antibodies that bind immunospecifically to the substances of the invention for use in therapeutic or diagnostic methods. These antibodies may be generated according to methods known in the art, using prediction from hydrophobicity charts, as described in the “Anti-NOVX Antibodies” section below.

NOV96

NOV96 includes three monocarboxylate transporter-like proteins, designated herein as NOV96a, NOV96b and NOV96c.

NOV96a

The disclosed NOV96a (alternatively referred to herein as CG56918-01) includes the 1302 nucleotide sequence (SEQ ID NO:301) shown in Table 96A. A NOV96a ORF begins with a Kozak consensus ATG initiation codon at nucleotides 9-11 and ends with a stop codon at nucleotides 1287-1289. The disclosed NOV96a maps to human chromosome 17.

TABLE 96A
NOV96a Nucleotide Sequence

| | |
|---|---|
| CCGCTTAGATGGCGCGCAGGACAGAGCCCCCCGACGGGGGCTGGGGATGGGTGGTGGTGC | (SEQ ID NO:301) |
| TCTCAGCGTTCTTCCAGTCGGCGCTTGTGTTTGGGGTGCTCCGCTCCTTTGGGGTCTTCT | |
| TCGTGGAGTTTGTGGCGGCGTTTGAGGAGCAGGCAGCGCGCGTCTCCTGGATCGCCTCCA | |
| TAGGAATCGCGGTGCAGCAGTTTGGGAGCCCGGTAGGCAGTGCCCTGAGCACGAAGTTCG | |
| GGCCCAGGCCCGTGGTGATGACTGGAGGCATCTTGGCTGCGCTGGGGATGCTGCTCGCCT | |
| CTTTTGCTACTTCCTTGACCCACCTATACCTGAGTATTGGGTTGCTGTCAGGCTCTGGCT | |
| GGGCTTTGACCTTCGCTCCGACCCTGGCCTGCCTGTCCTGTTATTTTTCTCGCCGACGAT | |
| CCCTGGCCACCGGGCTGGCACTGACAGGCGTGGGCCTCTCCTCCTTCACATTTGCCCCCT | |
| TTTTCCAGTGGCTGCTCAGCCACTACGCCTGGAGGGGGTCCCTGCTGCTGGTGTCTGCCC | |
| TCTCCCTCCACCTAGTGGCCTGTGGTGCTCTCCTCCGCCCACCCTCCCTGGCTGAGGACC | |
| CTGCTGTGGGTGGTCCCAGGGCCCAACTCACCTCTCTCCTCCATCATGGCCCCTTCCTCC | |
| GTTACACTGTTGCCCTCACCCTGATCAACACTGGCTACTTCATTCCCTACCTCCACCTGG | |
| TGGCCCATCTCCAGGACCTGGATTGGGACCCACTACCTGCTGCCTTCCTACTCTCAGTTG | |
| TTGCTATTTCTGACCTCGTGGGGCGTGTGGTCTCCGGATGGCTGGGAGATGCAGTCCCAG | |
| GGCCTGTGACACGACTCCTGATGCTCTGGACCACCTTGACTGGGGTGTCACTAGCCCTGT | |
| TCCCTGTAGCTCAGGCTCCCACAGCCCTGGTGGCTCTGGCTGTGGCCTACGGCTTCACAT | |
| CAGGGGCTCTGGCCCCACTGGCCTTCTCCGTGCTGCCTGAACTAATAGGGACTAGAAGGA | |
| TTTACTGTGGCCTGGGACTGTTGCAGATGGTAGAGAGCATCGGGGGGCTGCTGGGGCCTC | |
| CTCTCTCAGGCTACCTCCGGGATGTGACAGGCAACTACACGGCTTCTTTTGTGGTGGCTG | |
| GGGCCTTCCTTCTTTCAGGGAGTGGCATTCTCCTCACCCTGCCCCACTTCTTCTGCTCCT | |
| CAACTACTACCTCCGGGCCCCAGGACCTTGTAACAGAAGCACTAGATACTAAAGTTCCCC | |
| TACCCAAGGAGGGGCTGGAAGAGGACTGAACTCCACAGAGTC | |

A NOV96a polypeptide (SEQ ID NO:302) encoded by SEQ ID NO:301 is 426 amino acids in length and is presented using the one-letter amino acid code in Table 96B. The Psort profile for NOV96a predicts that this sequence is a Type IIIa membrane protein, has a signal peptide, and is likely to be localized to the plasma membrane with a certainty of 0.6400. In alternative embodiments, a NOV96a polypeptide is located to the Golgi with a certainty of 0.4600, or to the endoplasmic reticulum (membrane) with a certainty of 0.3700. The Signal P predicts a likely cleavage site for a NOV96a peptide is between positions 34 and 35, i.e., at the dash in the sequence VAA-FE.

TABLE 96B
NOV96a Polypeptide Sequence

| | |
|---|---|
| MARRTEPPDGGWGWVVVLSAFFQSALVFGVLRSFGVFFVEFVAAFEEQAARVSWIASIGI | (SEQ ID NO:302) |
| AVQQFGSPVGSALSTKFGPRPVVMTGGILAALGMLLASFATSLTHLYLSIGLLSGSGWAL | |
| TFAPTLACLSCYFSRRRSLATGLALTGVGLSSFTFAPFFQWLLSHYAWRGSLLLVSALSL | |
| HLVACGALLRPPSLAEDPAVGGPRAQLTSLLHHGPFLRYTVALTLINTGYFIPYLHLVAH | |
| LQDLDWDPLPAAFLLSVVAISDLVGRVVSGWLGDAVPGPVTRLLMLWTTLTGVSLALFPV | |
| AQAPTALVALAVAYGFTSGALAPLAFSVLPELIGTRRIYCGLGLLQMVESIGGLLGPPLS | |
| GYLRDVTGNYTASFVVAGAFLLSGSGILLTLPHFFCSSTTTSGPQDLVTEALDTKVPLPK | |
| EGLEED | |

NOV96b

The disclosed NOV96b (alternatively referred to herein as CG56918-02) includes the 1294 nucleotide sequence (SEQ ID NO:303) shown in Table 96C. A SEC2 ORF begins with a Kozak consensus ATG initiation codon at nucleotides 1-3 and ends with a TGA codon at nucleotides 1279-1281. The disclosed NOV96b maps to human chromosome 17.

TABLE 96C
NOV96b Nucleotide Sequence

| | |
|---|---|
| ATGGCGCGCAGGACAGAGCCCCCCGACGGGGGCTGGGGATGGGTGGTGGTGCTCTCAGCG | (SEQ ID NO:303) |
| TTCTTCCAGTCGGCGCTTGTGTTTGGGGTGCTCCGCTCCTTTGGGGTCTTCTTCGTGGAG | |
| TTTGTGGCGGCGTTTGAGGAGCAGGCAGCGCGCGTCTCCTGGATCGCCTCCATAGGAATC | |
| GCGGTGCAGCAGTTTGGGAGCCCGGTAGGCAGTGCCCTGAGCACGAAGTTCGGGCCCAGG | |
| CCCGTGGTGATGACTGGAGGCATCTTGGCTGCGCTGGGGATGCTGCTCGCCTCTTTTGCT | |
| ACTTCCTTGACCCACCTATACCTGAGTATTGGGTTGCTGTCAGGCTCTGGCTGGGCTTTG | |
| ACCTTCGCTCCGACCCTGGCCTGCCTGTCCTGTTATTTTTCTCGCCGACGATCCCTGGCC | |
| ACCGGGCTGGCACTGACAGGCGTGGGCCTCTCCTCCTTCACATTTGCCCCCTTTTTCCAG | |
| TGGCTGCTCAGCCACTACGCCTGGAGGGGGTCCCTGCTGCTGGTGTCTGCCCTCTCCCTC | |
| CACCTAGTGGCCTGTGGTGCTCTCCTCCGCCCACCCTCCCTGGCTGAGGACCCTGCTGTG | |
| GGTGGTCCCAGGGCCCAACTCACCTCTCTCCTCCATCATGGCCCCTTCCTCCGTTACACT | |
| GTTGCCCTCACCCTGATCAACACTGGCTACTTCATTCCCTACCTCCACCTGGTGGCCCAT | |
| CTCCAGGACCTGGATTGGGACCCACTACCTGCTGCCTTCCTACTCTCAGTTGTTGCTATT | |
| TCTGACCTCGTGGGGCGTGTGGTCTCCGGATGGCTGGGAGATGCAGTCCCAGGGCCTGTG | |
| ACACGACTCCTGATGCTCTGGACCACCTTGACTGGGGTGTCACTAGCCCTGTTCCCTGTA | |
| GCTCAGGCTCCCACAGCCCTGGTGGCTCTGGCTGTGGCCTACGGCTTCACATCAGGGGCT | |
| CTGGCCCCACTGGCCTTCTCCGTGCTGCCTGAACTAATAGGGACTAGAAGGATTTACTGT | |
| GGCCTGGGACTGTTGCAGATGGTAGAGAGCATCGGGGGGCTGCTGGGGCCTCCTCTCTCA | |
| GGCTACCTCCGGGATGTGACAGGCAACTACACGGCTTCTTTTGTGGTGGCTGGGGCCTTC | |
| CTTCTTTCAGGGAGTGGCATTCTCCTCACCCTGCCCCACTTCTTCTGCTCCTCAACTACT | |
| ACCTCCGGGCCCCAGGACCTTGTAACAGAAGCACTAGATACTAAAGTTCCCCTACCCAAG | |
| GAGGGACTGGAAGAGGACTGAACTCCACAGAGTC | |

A NOV96b polypeptide (SEQ ID NO:304) encoded by SEQ ID NO:303 is 426 amino acids in length and is presented using the one-letter amino acid code in Table 96D. The Psort profile for NOV96b predicts that this sequence is likely to be a Type IIIa membrane protein, has a signal peptide, and is likely to be localized to the plasma membrane with a certainty of 0.6400. In alternative embodiments, a NOV96b polypeptide is located to the Golgi with a certainty of 0.4600, or to the endoplasmic reticulum (membrane) with a certainty of 0.3700. The Signal P predicts a likely cleavage site for a NOV96b peptide is between positions 44 and 45, i.e., at the dash in the sequence VAA-FE.

TABLE 96D
NOV96b Polypeptide Sequence

| | |
|---|---|
| MARRTEPPDGGWGWVVVLSAFFQSALVFGVLRSFGVFFVEFVAAFEEQAARVSWIASIGI | (SEQ ID NO:304) |
| AVQQFGSPVGSALSTKFGPRPVVMTGGILAALGMLLASFATSLTHLYLSIGLLSGSGWAL | |
| TFAPTLACLSCYFSRRRSLATGLALTGVGLSSFTFAPFFQWLLSHYAWRGSLLLVSALSL | |
| HLVACGALLRPPSLAEDPAVGGPRAQLTSLLHHGPFLRYTVALTLINTGYFIPYLHLVAH | |
| LQDLDWDPLPAAFLLSVVAISDLVGRVVSGWLGDAVPGPVTRLLMLWTTLTGVSLALFPV | |
| AQAPTALVALAVAYGFTSGALAPLAFSVLPELIGTRRIYCGLGLLQMVESIGGLLGPPLS | |
| GYLRDVTGNYTASFVVAGAFLLSGSGILLTLPHFFCSSTTTSGPQDLVTEALDTKVPLPK | |
| EGLEED | |

NOV96c

The disclosed NOV96c (alternatively referred to herein as CG56918-03) includes the 1445 nucleotide sequence (SEQ ID NO:305) shown in Table 96E. A SEC2 ORF begins with a Kozak consensus ATG initiation codon at nucleotides 9-11 and ends with a stop codon at nucleotides 1287-1289. The disclosed NOV96c maps to human chromosome 17.

TABLE 96E
NOV96c Nucleotide Sequence

| | |
|---|---|
| CCGCTTAGATGGCGCGCAGGACAGAGCCCCCCGACGGGGGCTGGGGATGGGTGGTGGTGC | (SEQ ID NO:305) |
| TCTCAGCGTTCTTCCAGTCGGCGCTTGTGTTTGGGGTGCTCCGCTCCTTTGGGGTCTTCT | |
| TCGTGGAGTTTGTGGCGGCGTTTGAGGAGCAGGCAGCGCGCGTCTCCTGGATCGCCTCCA | |
| TAGGAATCGCGGTGCAGCAGTTTGGGAGCCCGGTAGGCAGTGCCCTGAGCACGAAGTTCG | |
| GGCCCAGGCCCGTGGTGATGACTGGAGGCATCTTGGCTGCGCTGGGGATGCTGCTCGCCT | |
| CTTTTGCTACTTCCTTGACCCACCTATACCTGAGTATTGGGTTGCTGTCAGGCTCTGGCT | |
| GGGCTTTGACCTTCGCTCCGAGCCTGGCCTGCCTGTCCTGTTATTTCTCTCGCCGACGAT | |
| CCCTGGCCACCGGGCTGGCACTGACAGGCGTGGGCCTCTCCTCCTTCACATTTGCCCCCT | |
| TTTTCCAGTGGCTGCTCAGCCACTACGCCTGGAGGGGGTCCCTGCTGCTGGTGTCTGCCC | |
| TCTCCCTCCACCTAGTGGCCTGTGGTGCTCTCCTCCGCCCACCCTCCCTGGCTGAGGACC | |
| CTGCTGTGGGTGGTCCCAGGGCCCAACTCACCTCTCTCCTCCATCATGGCCCCTTCCTCC | |
| GTTACACTGTTGCCCTCACCCTGATCAACACTGGCTACTTCATTCCCTACCTCCACCTGG | |
| TGGCCCATCTCCAGGACCTGGATTGGGACCCACTACCTGCTGCCTTCCTACTCTCAGTTG | |
| TTGCTATTTCTGACCTCGTGGGGCGTGTGGTCTCCGGATGGCTGGGAGATGCAGTCCCAG | |
| GGCCTGTGACACGACTCCTGATGCTCTGGACCACCTTGACTGGGGTGTCACTAGCCCTGT | |
| TCCCTGTAGCTCAGGCTCCCACAGCCCTGGTGGCTCTGGCTGTGGCCTACGGCTTCACAT | |
| CAGGGGCTCTGGCCCCACTGGCCTTCTCCGTGCTGCCTGAACTAATAGGGACTAGAAGGA | |
| TTTACTGTGGCCTGGGACTGTTGCAGATGATAGAGAGCATCGGGGGGCTGCTGGGGCCTC | |
| CTCTCTCAGGCTACCTCCGGGATGTGTCAGGCAACTACACGGCTTCTTTTGTGGTGGCTG | |
| GGGCCTTCCTTCTTTCAGGGAGTGGCATTCTCCTCACCCTGCCCCACTTCTTCTGCTTCT | |
| CAACTACTACCTCCGGGCCTCAGGACCTTGTAACAGAAGCACTAGATACTAAAGTTCCCC | |
| TACCCAAGGAGGGGCTGGAAGAGGACTGAACTCCACAGAGTCAGGCCCAGAAAGCCAAAG | |
| CTTGACAGCTCCAGGTCTTCTCTTGCCACGTCTTGGTCTCCACAGAACCACAGTGCCTTA | |
| AGATTCTTGATCTGCCTCCCCCTAGAGCAGGCCTGGGGCTCCTGCAATGTGTGTGCCAAC | |
| CCTTT | |

The NOV96c polypeptide (SEQ ID NO:306) encoded by SEQ ID NO:305 is 426 amino acids in length and is presented using the one-letter amino acid code in Table 96F. The Psort profile for NOV96c predicts that this sequence is a Type III a membrane protein, has a signal peptide, and is likely to be localized to the plasma membrane with a certainty of 0.6400. In alternative embodiments, a NOV96c polypeptide is located to the Golgi with a certainty of 0.4600, or to the endoplasmic reticulum (membrane) with a certainty of 0.3700. The Signal P predicts a likely cleavage site for a NOV96c peptide is between positions 34 and 35, i.e., at the dash in the sequence VAA-FE.

TABLE 96F
NOV96c Polypeptide Sequence

| | |
|---|---|
| MARRTEPPDGGWGWVVVLSAFFQSALVFGVLRSFGVFFVEFVAAFEEQAARVSWIASIGI | (SEQ ID NO:306) |
| AVQQFGSPVGSALSTKFGPRPVVNTGGILAALGMLLASFATSLTHLYLSIGLLSGSGWAL | |
| TFAPSLACLSCYFSRRRSLATGLALTGVGLSSFTFAPFFQWLLSHYAWRGSLLLVSALSL | |
| HLVACGALLRPPSLAEDPAVGGPRAQLTSLLHHGPFLRYTVALTLINTGYFIPYLHLVAH | |
| LQDLDWDPLPAAFLLSVVAISDLVGRVVSGWLGDAVPGPVTRLLMLWTTLTGVSLALFPV | |
| AQAPTALVALAVAYGFTSGALAPLAFSVLPELIGTRRIYCGLGLLQMIESIGGLLGPPLS | |
| GYLRDVSGNYTASFVVAGAFLLSGSGILLTLPHFFCFSTTTSGPQDLVTEALDTKVPLPK | |
| EGLEED | |

A BLAST analysis of NOV96 was run against the proprietary PatP GENESEQ Protein Patent database. It was found, for example, that the amino acid sequence of NOV96a had high homology to other proteins as shown in Table 96G.

TABLE 96G
BLASTX results from PatP database for NOV96a

| | | Smallest |
|---|---|---|
| | | Sum |
| | High | Probability |
| Sequences producing High-scoring Segment Pairs: | Score | P(N) |
| patp: AAU01618 Human secreted protein | 940 | 3.0e−94 |
| patp: AAM93737 Human polypeptide | 940 | 3.0e−94 |
| patp: AAB88570 Human hydrophobic | 620 | 2.5e−60 |
| domain containing protein | | |
| patp: AAY31642 Human transport-associated | 602 | 2.0e−58 |
| protein-4 | | |
| patp: AAU01586 Human secreted protein | 357 | 1.8e−32 |
| related to gene #26 | | |

In a search of sequence databases, it was found, for example, that the nucleic acid sequence of this invention has 420 of 702 bases (59%) identical to a gb:GENBANK-ID:RNU87627|acc:U87627.1 mRNA from Rattus norvegicus (Rattus norvegicus putative monocarboxylate transporter (MCT3) mRNA). The full amino acid sequence of the protein of the invention was found to have 89 of 191 amino acid residues (46%) identical to, and 119 of 191 amino acid residues (62%) similar to, the 504 amino acid residue ptnr:SPTREMBL-ACC:095907 protein from Homo sapiens (Human) (DJ1039K5.2 (SIMILAR TO MONOCARBOXYLATE TRANSPORTER (MCT3)). NOV96 also has homology to the other proteins shown in the BLASTP data in Table 96H.

TABLE 96H
NOV96 BLASTP results

| Gene Index/ | | Length | Identity | Positive | |
|---|---|---|---|---|---|
| Identifier | Protein/Organism | (aa) | (%) | (%) | Expect |
| gi\|17491104\|ref\| | similar to solute | 427 | 424/427 | 425/427 | 0.0 |
| XP_064368.1\| | carrier family 16 | | (99) | (99) | |
| (XM_064368) | (monocarboxylic acid | | | | |
| | transporters), member 8 | | | | |
| | (H. sapiens) | | | | |
| | [Homo sapiens] | | | | |
| gi\|14754801\|ref\| | solute carrier family | 478 | 141/410 | 213/410 | 2e−57 |
| XP_044738.1\| | 16 (monocarboxylic acid | | (34) | (51) | |
| (XM_044738) | transporters), member 7 | | | | |
| | [Homo sapiens] | | | | |
| gi\|4759120\|ref\| | solute carrier family | 478 | 141/410 | 213/410 | 2e−57 |
| NP_004722.1\| | 16 (monocarboxylic acid | | (34) | (51) | |
| (NM_004731) | transporters), member | | | | |
| | 7; monocarboxylate | | | | |
| | transporter 2 | | | | |
| | [Homo sapiens] | | | | |
| gi\|2497855\|sp\| | MONOCARBOXYLATE | 489 | 140/410 | 215/410 | 7e−57 |
| Q63344\|MOT2_RAT | TRANSPORTER 2 (MCT 2) | | (34) | (52) | |
| gi\|1432167\|gb\| | monocarboxylate | 489 | 141/410 | 215/410 | 8e−57 |
| AAB04023.1\| | transporter 2 | | (34) | (52) | |
| (U62316) | [Rattus norvegicus] | | | | |

This BLASTP data is displayed graphically in the ClustalW in Table 96I. A multiple sequence alignment is given, with the NOV96a, b, and c proteins being shown on lines 1, 2, and 3 in a ClustalW analysis comparing the protein of the invention with the related protein sequences shown in Table 96H.

[image]

[image]

[image]

Monocarboxylates such as lactate and pyruvate play a central role in cellular metabolism and metabolic communication between tissues. Essential to these roles is their rapid transport across the plasma membrane, which is catalysed by a recently identified family of proton-linked monocarboxylate transporters (MCTs). Nine MCT-related sequences have so far been identified in mammals, each having a different tissue distribution, whereas six related proteins can be recognized in Caenorhabditis elegans and four in Saccharomyces cerevisiae.

Direct demonstration of proton-linked lactate and pyruvate transport has been demonstrated for mammalian MCT1-MCT4, but only for MCT1 and MCT2 have detailed analyses of substrate and inhibitor kinetics been described following heterologous expression in Xenopus oocytes. MCT1 is ubiquitously expressed, but is especially prominent in heart and red muscle, where it is up-regulated in response to increased work, suggesting a special role in lactic acid oxidation. By contrast, MCT4 is most evident in white muscle and other cells with a high glycolytic rate, such as tumour cells and white blood cells, suggesting it is expressed where lactic acid efflux predominates. MCT2 has a ten-fold higher affinity for substrates than MCT1 and MCT4 and is found in cells where rapid uptake at low substrate concentrations may be required, including the proximal kidney tubules, neurons and sperm tails. MCT3 is uniquely expressed in the retinal pigment epithelium. MCT1 and MCT4 have been shown to interact specifically with OX-47 (CD147), a member of the immunoglobulin superfamily with a single transmembrane helix. This interaction appears to assist MCT expression at the cell surface (Halestrap and Price, 1999, Biochem. J. vol.343: 281-99).

NOV96 is predicted to be expressed in at least the following tissues: adrenal gland, bone marrow, brain—amygdala, brain—cerebellum, brain—hippocampus, brain—substantia nigra, brain—thalamus, brain—whole, fetal brain, fetal kidney, fetal liver, fetal lung, foreskin, heart, kidney, lymphoma—Raji, mammary gland, pancreas, pituitary gland, placenta, prostate, salivary gland, skeletal muscle, small intestine, spinal cord, spleen, stomach, testis, thyroid, trachea and uterus. This information was derived by determining the tissue sources of the sequences that were included in the invention including but not limited to SeqCalling sources, public EST sources, literature sources, and/or RACE sources. Further expression data for NOV96 is provided in Example 2.

The NOV96 nucleic acids and proteins are useful in potential therapeutic applications implicated in various pathological disorders described further herein, for example, Von Hippel-Lindau (VHL) syndrome, Alzheimer's disease, stroke, tuberous sclerosis, hypercalceimia, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, neuroprotection, hemophilia, hypercoagulation, idiopathic thrombocytopenic purpura, immunodeficiencies, hemophilia, hypercoagulation, immunodeficiencies, graft vesus host disease as well as other diseases, disorders and conditions. NOV96 nucleic acids encoding the monocarboxylate transporter-like protein of the invention, or fragments thereof, may further be useful in diagnostic applications, wherein the presence or amount of the nucleic acid or the protein are to be assessed.

The novel nucleic acid of the invention encoding a monocarboxylate transporter-like protein includes the nucleic acid whose sequence is provided in Table 96A, 105C, or 105E, or a fragment thereof. The invention also includes a mutant or variant nucleic acid any of whose bases may be changed from the corresponding base shown in Table 96A, 105C, or 105E while still encoding a protein that maintains its monocarboxylate transporter-like activities and physiological functions, or a fragment of such a nucleic acid. The invention further includes nucleic acids whose sequences are complementary to the sequence of Table 96A, 105C, or 105E, including nucleic acid fragments that are complementary to any of the nucleic acids just described. The invention additionally includes nucleic acids or nucleic acid fragments, or complements thereto, whose structures include chemical modifications. Such modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 41% of the bases may be so changed.

The novel protein of the invention includes the monocarboxylate transporter-like protein whose sequence is provided in Table 96B, 105D, or 105F. The invention also includes a mutant or variant protein any of whose residues may be changed from the corresponding residue shown in Table 96B, 105D, or 105F while still encoding a protein that maintains its monocarboxylate transporter-like activities and physiological functions, or a functional fragment thereof. In the mutant or variant protein, up to about 54% of the amino acid residues may be so changed.

These materials are further useful in the generation of antibodies that bind immunospecifically to the substances of the invention for use in therapeutic or diagnostic methods. These antibodies may be generated according to methods known in the art, using prediction from hydrophobicity charts, as described in the “Anti-NOVX Antibodies” section below.

NOV97

NOV97 includes six carboxypeptidase-like proteins, designated herein as NOV97a, NOV97b, NOV97c, NOV97d, NOV97e, and NOV97f.

NOV97a

The disclosed NOV97a (alternatively referred to herein as CG57070-01) includes the 1279 nucleotide sequence (SEQ ID NO:307) shown in Table 97A. A NOV97a ORF begins with a Kozak consensus ATG initiation codon at nucleotides 1-3 and ends with a stop codon at nucleotides 1258-1260. The disclosed NOV97a maps to human chromosome 7q 31.

TABLE 97A
NOV97a Nucleotide Sequence

| | |
|---|---|
| ATGCGGGGGTTGCTGGTGTTGAGTGTCCTGTTGCGGGCTGTCTTTGGCAAGGAGGACTTT | (SEQ ID NO:307) |
| GTGGGGCATCAGGTGCTCCGAATCTCTGTAGCCGATGACGCCCAGGTACACAAGGTGAAG | |
| GAGCTGGAGGACCTGGAGCACCTGCAGCTGGACTTCTGGCGGGGGCCTGCCCACCCTGGC | |
| TCCCCCATCGACGTCCGAGTGCCCTTCCCCAGCATCCAGGCGGTCAAGATCTTTCTGGAG | |
| TCCCACGGCATCAGCTATGAGACCATGATCGAGGACGTGCAGTCGCTGCTGGACGAGGAG | |
| CAGGAGCAGATGTTCGCCTTCCGGTCCCGGGCGCGCTCCACCGACACTTTTAACTACGCC | |
| ACCTACCACACCCTGGAGGAGGTGTATAGCTGGATTGACAACTTTGTAATGGAGCATTCC | |
| GATATTGTCTCAAAAATTCAGATTGGCAACAGCTTTGAAAACCAGTCCATTCTTGTCCTG | |
| AAGTTCAGCACTGGAGGTTCTCGGCACCCAGCCATCTGGATCGACACTGGAATTCACTCC | |
| CGGGAGTGGATCACCCATGCCACCGGCATCTGGACTGCCAATAAGATTGTCAGTGATTAT | |
| GGCAAAGACCGTGTCCTGACACACATACTGAATGCCATGGACATCTTCATAGAGCTCGTC | |
| ACAAACCCTGATGCGTTTGCTTTTACCCACAGCATGAACCGCTTATGGCGGAAGAACAAG | |
| TCCATCAGACCTGGAATCTTCTGCATCGGCGTGGATCTCAACAGGAACTGGAAGTCGGGT | |
| TTTGGAGGAAATGGTTCTAACAGCAACCCCTGCTCAGAAACTTATCACGGGCCCTCCCCT | |
| CAGTCGGAGCCGGAGGTCGCTGCCATAGTGAACTTCATCACAGCCCATGGCAACTTCAAG | |
| GCTCTGATCTCCATCCACAGCTACTCTCAGATGCTTATGTACCCTTACGGCCGATTGCTG | |
| GAGCCCGTTTCAAATCAGAGGGAGTTGTACGATCTTGCCAAGGATGCGGTGGAGGCCTTG | |
| TATAAGGTCCATGGCATCGAGTACATTTTTGGCAGCATCAGCACCACCCTCTATGTGGCC | |
| AGTGCGATCACCGTCGACTGGGCCTATGACAGTGGCATCAAGTACGCCTTCAGCTTTGAG | |
| CTCCGGGACACTGGGCAGTATGGCTTCCTGCTGCCGGCCACACAGATCATCCCCACGGCC | |
| CAGGAGACGTGGATGGCGCTTCGGACCATCATGGAGCACACCCTGAATCACCCCTACTAG | |
| CAGCACGACTGAGGGCAGG | |

A NOV97a polypeptide (SEQ ID NO:308) encoded by SEQ ID NO:307 is 419 amino acids in length and is presented using the one-letter amino acid code in Table 97B. The Psort profile for NOV97a predicts that this sequence has a signal peptide and is likely to be localized outside the cell with a certainty of 0.3703. In alternative embodiments, a NOV97a polypeptide is located to lysosomes with a certainty of 0.46200. The Signal P predicts a likely cleavage site for a NOV97a peptide is between positions 16 and 17, i.e., at the dash in the sequence VFG-KE.

TABLE 97B
NOV97a Polypeptide Sequence

| | |
|---|---|
| MRGLLVLSVLLGAVFGKEDFVGHQVLRISVADEAQVQKVKELEDLEHLQLDFWRGPAHPG | (SEQ ID NO:308) |
| SPIDVRVPFPSIQAVKIFLESHGISYETMIEDVQSLLDEEQEQMFAFRSRARSTDTFNYA | |
| TYHTLEEVYSWIDNFVMEHSDIVSKIQIGNSFENQSILVLKFSTGGSRHPAIWIDTGIHS | |
| REWITHATGIWTANKIVSDYGKDRVLTDILNAMDIFIELVTNPDGFAFTHSMNRLWRKNK | |
| SIRPGIFCIGVDLNRNWKSGFGGNGSNSNPCSETYHGPSPQSEPEVAAIVNFITAHGNFK | |
| ALISIHSYSQMLMYPYGRLLEPVSNQRELYDLAKDAVEALYKVHGIEYIFGSISTTLYVA | |
| SGITVDWAYDSGIKYAFSFELRDTGQYGFLLPATQIIPTAQETWMALRTIMEHTLNHPY | |

NOV97b

The disclosed NOV97b (alternatively referred to herein as CG57070-02) includes the 1291 nucleotide sequence (SEQ ID NO:309) shown in Table 97C. A SEC2 ORF begins with a Kozak consensus ATG initiation codon at nucleotides 1-3 and ends with a stop codon at nucleotides 1270-1272. The disclosed NOV97b maps to human chromosome 7q31.

TABLE 97C
NOV97b Nucleotide Sequence

| | |
|---|---|
| ATGCGGGGGTTGCTGGTGTTGAGTGTCCTGTTGGGGGCTGTCTTTGGCAAGGAGGACTTT | (SEQ ID NO:309) |
| GTGGGGCATCAGGTGCTCCGAATCTCTGTAGCCGATGAGGCCCAGGTACAGAAGGTGAAG | |
| GAGCTGGAGGACCTGGAGCACCTGCAGGTGCACTTCTGGCGTGGCCCAGCCAGGCCCAGC | |
| CTCCCTCTGGATATGAGAGTTCCTTTCTCTGAACTGAAAGACATCAAAGCTTATCTGGAG | |
| TCTCATGGACTTGCTTACAGCATCATGATAAAGGACATCCAGGTGAAGCCCCAGGTGCTG | |
| CTGGATGAGGAAAGACAGGCCATGGCGAAATCCCGCCGGCTGGAGCGCAGCACCAACAGC | |
| TTCAGTTACTCATCATACCACACCCTGGAGGAGGTATATAGCTGGATTGACAACTTTGTA | |
| ATGGAGCATTCCGATATTGTCTCAAAAATTCAGATTGGCAACAGCTTTGAAAACCAGTCC | |
| ATTCTTGTCCTGAAGTTCAGCACTGGAGGTTCTCGGCACCCAGCCATCTGGATCGACACT | |
| GGAATTCACTCCCGGGAGTGGATCACCCATGCCACCGGCATCTGGACTGCCAATAAGATT | |
| GTCAGTGATTATGGCAAAGACCGTGTCCTGACAGACATACTGAATGCCATGGACATCTTC | |
| ATAGAGCTCGTCACAAACCCTGATGGGTTTGCTTTTACCCACAGCATGAACCGCTTATGG | |
| CGGAAGAACAAGTCCATCAGACCTGGAATCTTCTGCATCGGCGTGGATCTCAACAGGAAC | |
| TGGAAGTCGGGTTTTGGAGGAAATGGTTCTAACAGCAACCCCTGCTCAGAAACTTATCAC | |
| GGGCCCTCCCCTCAGTCGGAGCCGGAGGTGGCTGCCATAGTGAACTTCATCACAGCCCAT | |
| GGCAACTTCAAGGCTCTGATCTCCATCCACAGCTACTCTCAGATGCTTATGTACCCTTAC | |
| GGCCGATTGCTGGAGCCCGTTTCAAATCAGAGGGAGTTGTACGATCTTGCCAAGGATGCG | |
| GTGGAGGCCTTGTATAAGGTCCATGGGATCGAGTACATTTTTGCCAGCATCAGCACCACC | |
| CTCTATGTGGCCAGTGGGATCACCGTCGACTGGGCCTATGACAGTGGCATCAAGTACGCC | |
| TTCAGCTTTGAGCTCCGGGACACTGGCCAGTATGCCTTCCTGCTGCCGGCCACACAGATC | |
| ATCCCCACGGCCCAGGAGACGTGGATGGCGCTTCGGACCATCATGGAGCACACCCTGAAT | |
| CACCCCTACTAGCAGCACGACTGAGGGCAGG | |

A NOV97b polypeptide (SEQ ID NO:310) encoded by SEQ ID NO:309 is 423 amino acids in length and is presented using the one-letter amino acid code in Table 97D. The Psort profile for NOV97b predicts that this sequence has a signal peptide and is likely to be localized outside the cell with a certainty of 0.3703. In alternative embodiments, a NOV97b polypeptide is located to lysosomes with a certainty of 0.1900. The Signal P predicts a likely cleavage site for a NOV97b peptide is between positions 16 and 17, i.e., at the dash in the sequence VFG-KE.

TABLE 97D
NOV97b Polypeptide Sequence

| | |
|---|---|
| MRGLLVLSVLLGAVFGKEDFVGHQVLRISVADEAQVQKVKELEDLEHLQVDFWRGPARPS | (SEQ ID NO:310) |
| LPVDMRVPFSELKDIKAYLESHGLAYSIMIKDIQVKPQVLLDEERQANAKSRRLERSTNS | |
| FSYSSYHTLEEVYSWIDNFVMEHSDIVSKIQIGNSFENQSILVLKFSTGGSRHPAIWIDT | |
| GIHSREWITHATGIWTANKIVSDYGKDRVLTDILNAMDIFIELVTNPDGFAFTHSMNRLW | |
| RRNKSIRPGIFCIGVDLNRNWKSGFGGNGSNSNPCSETYHGPSPQSEPEVAAIVNFITAH | |
| GNFKALISIHSYSQMLMYPYGRLLEPVSNQRELYDLAKDAVEALYKVHGIEYIFGSISTT | |
| LYVASGITVDWAYDSGIKYAFSFELRDTGQYGFLLPATQIIPTAQETWMALRTIMEHTLN | |
| HPY | |

NOV97c

The disclosed NOV97c (alternatively referred to herein as CG57070-03) includes the 1344 nucleotide sequence (SEQ ID NO:311) shown in Table 97E. A NOV97a ORF begins with a Kozak consensus ATG initiation codon at nucleotides 25-27 and ends with a stop codon at nucleotides 1327-1329.

TABLE 97E
NOV97c Nucleotide Sequence

| | |
|---|---|
| TGAAGCTCACCAGGAGGAAGAAGCATGCAGGGCACCCCTGGAGGCGGGACGCGCCCTGGG | (SEQ ID NO:311) |
| CCATCCCCCGTGGACAGGCGGACACTCCTGGTCTTCAGCTTTATCCTGGCAGCAGCTTTG | |
| GGCCAAATGAATTTCACAGGGCAGGTTCTTCGAGTCCTGGCCAAAGATGAGAAGCAGCTT | |
| TCACTTCTCGGGGATCTGGACGGCCTGAAACCCCAGAAGGTGGACTTCTGGCGTGGCCCA | |
| GCCAGGCCCAGCCTCCCTGTGGATATGAGAGTTCCTTTCTCTGAACTGAAAGACATCAAA | |
| GCTTATCTGGAGTCTCATGGACTTGCTTACAGCATCATGATAAAGGACATCCAGGTGCTG | |
| CTGGATGAGGAAAGACAGGCCATGGCGAAATCCCGCCGGCTGCAGCGCAGCACCAACAGC | |
| TTCAGTTACTCATCATACCACACCCTGGAGGAGATATATAGCTGGATTGACAACTTTGTA | |
| ATGGAGCATTCCGATATTGTCTCAAAAATTCAGATTGGCAACAGCTTTGAAAACCAGTCC | |
| ATTCTTGTCCTGAAGTTCAGCACTGGAGGTTCTCGGCACCCAGCCATCTGGATTGACACT | |
| GGAATTCACTCCCCGGAGTGGATCACCCATGCCACCGGCATCTGGACTGCCAATAAGATT | |
| GTCAGTGATTATGCCAAAGACCGTGTCCTGACAGACATACTGAATGCCATGGACATCTTC | |
| ATAGAGCTCGTCACAAACCCTGATGGGTTTGCTTTTACCCACAGCATGAACCGCTTATGG | |
| CGGAAGAACAAGTCCATCAGACCTGGAATCTTCTGCATCGCCGTGGATCTCAACAGGAAC | |
| TGGAAGTCGGGTTTTGGAAATGGTTCTAACAGCAACCCCTGCTCAGAAACTTATCACGGG | |
| CCCTCCCCTCAGTCGGAGCCGGAGGTGGCTGCCATAGTGAACTTCATCACAGCCCATGGC | |
| AACTTCAAGGCTCTGATCTCCATCCACAGCTACTCTCAGATGCTTATGTACCCTTACGGC | |
| CGATTGCTGGAGCCCGTTTCAAATCAGAGGGAGTTGTACGATCTTGCCAAGGATGCGGTG | |
| GAGGCCTTGTATAAGGTCCATGCGATCGAGTACATTTTTGGCAGCATCAGCACCACCCTC | |
| GATGTGGCCAGTGGGATCACCGTCGACTGGGCCTATGACAGTGGCATCAAGTACGCCTTC | |
| AGCTTTGAGCTCCGGGACACTGGGCAGTATGGCTTCCTGCTGCCGGCCACACAGATCATC | |
| CCCACGGCCCACGAGACGTGGATGGCGCTTCGGACCATCATGGAGCACACCCTGAATCAC | |
| CCCTACTAGCAGCACGACTGAGGG | |

A NOV97c polypeptide (SEQ ID NO:312) encoded by SEQ ID NO:311 is 434 amino acids in length and is presented using the one-letter amino acid code in Table 97F. The Psort profile for NOV97c predicts that this sequence has a signal peptide and is likely to be localized outside the cell with a certainty of 0.5851. In alternative embodiments, a NOV97c polypeptide is located to lysosomes with a certainty of 0.4366. The Signal P predicts a likely cleavage site for a NOV97c peptide is between positions 33 and 34, i.e., at the dash in the sequence ALG-QM.

TABLE 97F
NOV97c Polypeptide Sequence

| | |
|---|---|
| MQGTPGGGTRPGPSPVDRRTLLVFSFILAAALGQMNFTGQVLRVLAKDEKQLSLLGDLEG | (SEQ ID NO:312) |
| LKPQKVDFWRGPARPSLPVDMRVPFSELKDIKAYLESHGLAYSIMIKDIQVLLDEERQAM | |
| AKSRRLERSTNSFSYSSYHTLEEIYSWIDNFVMEHSDIVSKIQIGNSFENQSILVLKFST | |
| GGSRHPAIWIDTGIHSREWITHATGIWTANKIVSDYGKDRVLTDILNAMDIFIELVTNPD | |
| GFAFTHSMNRLWRKNKSTRPGIFCIGVDLNRNWKSGFGNGSNSNPCSETYHGPSPQSEPE | |
| VAAIVNFITAHGNFKALISIHSYSQMLMYPYGRLLEPVSNQRELYDLAKDAVEALYKVHG | |
| IEYIFGSISTTLDVASGITVDWAYDSGIKYAFSFELRDTGQYGFLLPATQIIPTAQETWM | |
| ALRTIMEHTLNHPY | |

NOV97d

The disclosed NOV97d (alternatively referred to herein as CG57070-04) includes the 988 nucleotide sequence (SEQ ID NO:313) shown in Table 97G. A SEC2 ORF begins with a Kozak consensus ATG initiation codon at nucleotides 1-3 and ends with a TGA codon at nucleotides 973-975. The disclosed NOV97d maps to human chromosomes 7q31.

TABLE 97G
NOV97d Nucleotide Sequence

| | |
|---|---|
| ATGCGGGGGTTGCTGGTGTTGAGTGTCCTGTTGGGGGCTGTCTTTGGCAAGGAGGACTTT | (SEQ ID NO:313) |
| GTGGGGCATCAGGTGCTCCGAATCTCTGTAGCCGATGAGGCCCAGGTACAGAAGGTGAAG | |
| GAGCTGGACGACCTGGAGCACCTGCAGCTGGACTTCTGGCGGGGGCCTGCCCACCCTGGC | |
| TCCCCCATCGACGTCCGAGTGCCCTTCCCCAGCATCCAGGCGGTCAAGATCTTTCTGGAG | |
| TCCCACGGCATCAGCTATGAGACCATGATCGAGCACGTGCAGTCGCTGCTGGACGAGGAG | |
| CAGGAGCAGATGTTCGCCTTCCGGTCCCGGGCGCGCTCCACCGACACTTTTAACTACGCC | |
| ACCTACCACACCCTGGAGGAGGTGTATAGCTGGATTGACAACTTTGTAATGGAGCATTCC | |
| GATATTGTCTCAAAAATTCAGATTCGCAACAGCTTTGAAAACCAGTCCATTCTTGTCCTG | |
| AAGTTCAGCACTGGAGGTTCTCGGCACCCAGCCATCTGGATTGACACTGGAATTCACTCC | |
| CGGGAGTGGATCACCCATGCCACCGGCATCTGGACTGCCAATAAGATTGTCAGTGATTAT | |
| GGCAAAGACCGTGTCCTGACAGACATACTGAATGCCATGGACATCTTCATAGAGCTCGTC | |
| ACAAACCCTGATGGGTTTGCTTTTACCCACAGCATGAACCGCTTATGGCGGAAGAACAAG | |
| TCCATCAGACCTGGAATCTTCTGCATCGGCGTGGATCTCAACAGGAACTGGAAGTCGGGT | |
| TTTGGAGATGTGGCCAGTGCGATCACCGTCGACTGGGCCTACGACAGTGGCATCAAGTAC | |
| GCCTTCAGCTTTGAGCTCCGGGACACTGGGCAGTATGGCTTCCTGCTGCCGGCCACACAG | |
| ATCATCCCCACGGCCCAGGAGACGTGGATGGCGCTTCGGACCATCATGGAGCACACCCTG | |
| AATCACCCCTACTAGCAGCACGACTGAG | |

A NOV97d polypeptide (SEQ ID NO:314) encoded by SEQ ID NO:313 is 324 amino acids in length and is presented using the one-letter amino acid code in Table 97H. The Psort profile for NOV97d predicts that this sequence has a signal peptide and is likely to be localized to lysosomes with a certainty of 0.4757, or outside the cell with a certainty of 0.3703. The Signal P predicts a likely cleavage site for a NOV97d peptide is between positions 16 and 17, i.e., at the dash in the sequence VFG-KE.

TABLE 97H
NOV97d Polypeptide Sequence

| | |
|---|---|
| MRGLLVLSVLLGAVFGKEDFVGHQVLRISVADEAQVQKVKELEDLEHLQLDFWRGPAHPG | (SEQ ID NO:314) |
| SPIDVRVPFPSIQAVKIFLESHGISYETMIEDVQSLLDEEQEQMFAFRSRARSTDTFNYA | |
| TYHTLEEVYSWIDNFVMEHSDIVSKIQIGNSFENQSILVLKFSTGGSRHPAIWIDTGIHS | |
| REWITHATGIWTANKTVSDYGKDRVLTDILNANDIFTELVTNPDGFAFTHSMNRLWRKNK | |
| SIRPGIFCIGVDLNRNWKSGFGDVASGITVDWAYDSGIKYAFSFELRDTGQYGFLLPATQ | |
| IIPTAQETWMALRTIMEHTLNHPY | |

NOV97e

The disclosed NOV97e (alternatively referred to herein as CG57070-05) includes the 1348 nucleotide sequence (SEQ ID NO:315) shown in Table 97I. A NOV97e ORF begins with a Kozak consensus ATG initiation codon at nucleotides 25-27 and ends with a stop codon at nucleotides 1333-1335. The disclosed NOV97e maps to human chromosome 7.

TABLE 97I
NOV97e Nucleotide Sequence

| | |
|---|---|
| TGAAGCTCACCAGCAGGAAGAAGCATGCAGGGTACTCCTCGAGGCGGGACGCGCCCTGGG | (SEQ ID NO:315) |
| CCATCCCCCGTGGACAGGCGGACACTCCTGGTCTTCAGCTTTATCCTGGCAGCAGCTTTG | |
| GGCCAAATGAATTTCACAGGGGACCAGGTTCTTCGAGTCCTGGCCAAAGATGAGAAGCAG | |
| CTTTCACTTCTCGGGGATCTGGAGGGCCTGAAACCCCAGAAGGTGGACTTCTGGCGTGGC | |
| CCAGCCAGGCCCAGCCTCCCTGTGGATATGAGAGTTCCTTTCTCCGAACTGAAAGACATC | |
| AAAGCTTATCTGGAGTCTCATGGACTTGCTTACAGCATCATGATAAAGGACATCCAGGTG | |
| CTGCTGGATGAGGAAAGACAGGCCATGGCGAAATCCCGCCGGCTGGAGCGCAGCACCAAC | |
| AGCTTCAGTTACTCATCATACCACACCCTGGAGCAGATATATAGCTGGATTGACAACTTT | |
| GTAATGGAGCATTCCGATATTGTCTCAAAAATTCAGATTGGCAACAGCTTTGAAAACCAG | |
| TCCATTCTTGTCCTGAAGTTCAGCACTGGAGGTTCTCGGCACCCAGCCATCTGGATCGAC | |
| ACTGGAATTCACTCCCGGGAGTGGATCACCCATGCCACCGGCATCTGGACTGCCAATAAG | |
| ATTGTCAGTGATTATGGCAAAGACCGTGTCCTGACAGACATACTGAATGCCATGGACATC | |
| TTCATAGAGCTCGTCACAAACCCTGATGGGTTTGCTTTTACCCACAGCATGAACCGCTTA | |
| TGGCGGAAGAACAAGTCCATCAGACCTGGAATCTTCTGCATCGGCGTGGATCTCAACAGG | |
| AACTGGAAGTCCGGTTTTGGAGGAAATGGTTCTAACAGCAACCCCTGCTCAGAAACTTAT | |
| CACGGGCCCTCCCCTCAGTCGGAGCCGGAGGTGGCTGCCATAGTGAACTTCATCACAGCC | |
| CATGGCAACTTCAAGGCTCTGATCTCCATCCACAGCTACTCTCAGATGCTTATGTACCCT | |
| TACGGCCGATTGCTGGAGCCCGTTTCAAATCAGAGGGAGTTGTACGATCTTGCCAAGGAT | |
| GCGGTGGAGGCCTTGTATAAGGTCCATGGGATCGAGTACATTTTTGGCAGCATCAGCACC | |
| ACCCTCTATGTGGCCAGTGGGATCACCGTCGACTGGGCCTATGACAGTGGCATCAAGTAC | |
| GCCTTCAGCTTTGAGCTCCGGGACACTGGGCAGTATGGCTTCCTGCTGCCGGCCACACAG | |
| ATCATCCCCACGGCCCAGGAGACGTGGATGGCGCTTCGGACCATCATCCAGCACAACCTG | |
| AATCACCCCTACTAGCAGCACGACTGAG | |

A NOV97e polypeptide (SEQ ID NO:316) encoded by SEQ ID NO:315 is 436 amino acids in length and is presented using the one-letter amino acid code in Table 97J. The Psort profile for NOV97e predicts that this sequence has a signal peptide and is likely to be localized outside the cell with a certainty of 0.5851. In alternative embodiments, a NOV97e polypeptide is located to lysosomes with a certainty of 0.4421. The Signal P predicts a likely cleavage site for a NOV97e peptide is between positions 33 and 34, i.e., at the dash in the sequence ALG-QM.

TABLE 97J
NOV97e Polypeptide Sequence

| | |
|---|---|
| MQGTPGGGTRPGPSPVDRRTLLVFSFILAAALGQMNFTGDQVLRVLAKDEKQLSLLGDLE | (SEQ ID NO:316) |
| GLKPQKVDFWRGPARPSLPVDMRVPFSELKDIKAYLESHGLAYSIMIKDIQVLLDEERQA | |
| MAKSRRLERSTNSFSYSSYHTLEETYSWIDNFVMEHSDIVSKIQIGNSFENQSILVLKFS | |
| TGGSRHPAIWIDTGIHSREWITHATGIWTANKIVSDYGKDRVLTDILNAMDIFIELVTNP | |
| DGFAFTHSMNRLWRKNKSIRPGIFCIGVDLNRNWKSGFGGNCSNSNPCSETYHGPSPQSE | |
| PEVAAIVNFITAHGNFKALISIHSYSQMLMYPYGRLLEPVSNQRELYDLAKDAVEALYKV | |
| HGIEYIFGSISTILYVASGITVDWAYDSGIKYAFSFELRDTGQYGFLLPATQIIPTAQET | |
| WMALRTIMEHNLNHPY | |

NOV97f

The disclosed NOV97f (alternatively referred to herein as CG57070-06) includes the 975 nucleotide sequence (SEQ ID NO:317) shown in Table 97K. A SEC2 ORF begins with a Kozak consensus ATG initiation codon at nucleotides 1-3 and ends with a TAG codon at nucleotides 973-975. The disclosed NOV97f maps to human chromosome 7q31.

TABLE 97K
NOV97f Nucleotide Sequence

| | |
|---|---|
| ATGCGGGGGTGGCTGGTGTTGAGTGTCCTGTTGGGGGCTGTCTTTGGCAAGGAGGACTTT | (SEQ ID NO:317) |
| GTGGGGCATCAGGTGCTCCGAATCTCTGTAGCCGATGAGGCCCAGGTACAGAAGGTGAAG | |
| GAGCTGGAGGACCTGGAGCACCTGCAGCTGGACTTCTGGCGGGGGCCTGCCCACCCTGGC | |
| TCCCCCATCGACGTCCGAQTGCCCTTCCCCAGCATCCAGGCGGTCAAGATCTTTCTGGAG | |
| TCCCACGGCATCAGCTATGAGACCATGATCGAGGACGTGCAGTCGCTGCTGGACGAGGAG | |
| CAGGAGCAGATGTTCGCCTTCCGGTCCCGGGCGCGCTCCACCGACACTTTTAACTACGCC | |
| ACCTACCACACCCTGGAGGAGGTGTATAGCTGGATTGACAACTTTCTAATGGAGCATTCC | |
| GATATTGTCTCAAATATTCAGATTGGCAACAGCTTTGAAAACCAGTCCATTCTTGTCCTC | |
| AAGTTCAGCACTGGAGGTTCTCGGCACCCAGCCATCTGGATCGACACTGGAATTCACTCC | |
| CGGGAGTGCATCACCCGTGCCACCGGCATCTGGACTGCCAATAAGATTGTCAGTGATTAT | |
| GGCAAAGACCGTGTCCTGACAGACATACTGAATGCCATGGACATCTTCATAGGGCTCGTC | |
| ACAAACCCTGATGGGTTTGCTTTTACCCACAGCATGAACCGCTTATGGCGGAAGAACAAG | |
| TCCATCAGACCTGGAATCTTCTGCATCGGCGTGGATCTCAACAGGAACTGGAAGTCGGGT | |
| TTTGGAGATGTGGCCAGTGCGATCACCGTCGACTGGGCCTATGACAGTGGCATCAAGTAC | |
| GCCTTCAGCTTTGAGCTCCGGGACACTGGGCAGTATGGCTTCCTGCTGCCGGCCACACAG | |
| ATCATCCCCACCGCCCAGGAGACGTGGATGGCGCTTCGGACCATCATGGAGCACATCCTG | |
| AATCACCCCTACTAG | |

A NOV97f polypeptide (SEQ ID NO:318) encoded by SEQ ID NO:317 is 324 amino acids in length and is presented using the one-letter amino acid code in Table 97L. The Psort profile for NOV97f predicts that this sequence has a signal peptide and is likely to be localized outside the cell with a certainty of 0.3989. In alternative embodiments, a NOV97f polypeptide is located to lysosomes with a certainty of 0.5061. The Signal P predicts a likely cleavage site for a NOV97f peptide is between positions 16 and 17, i.e., at the dash in the sequence VFG-KE.

TABLE 97L
NOV97f Polypeptide Sequence

| | |
|---|---|
| MRGLLVLSVLLGAVFGKEDFVGHQVLRISVADEAQVQKVKELEDLEHLQLDFWRGPAHPG | (SEQ ID NO:318) |
| SPIDVRVPFPSIQAVKIFLESHGISYETMIEDVQSLLDEEQEQMFAFRSRARSTDTFNYA | |
| TYHTLEEVYSWIDNFVMEHSDIVSNIQIGNSFENQSILVLKFSTGGSRHPAIWIDTGIHS | |
| REWITRATGIWTANKIVSDYGKDRVLTDILNANDIFIGLVTNPDGFAFTHSMNRLWRKNK | |
| SIRPGIFCIGVDLNRNWKSGFGDVASGITVDWAYDSGIKYAFSFELRDTGQYGFLLPATQ | |
| IIPTAQETWMALRTIMEHILNHPY | |

A BLAST analysis of NOV97 was run against the proprietary PatP GENESEQ Protein Patent database. It was found, for example, that the amino acid sequence of NOV97 had high homology to other proteins as shown in Table 97M.

TABLE 97M
BLASTX results from PatP database for NOV97

| | | Smallest |
|---|---|---|
| | | Sum |
| | High | Probability |
| Sequences producing High-scoring Segment Pairs: | Score | P(N) |
| patp: AAE01663 Novel human protease #2 | 1898 | 9.3e−196 |
| patp: AAB47565 Protease PRTS-7 | 1898 | 9.3e−196 |
| patp: AAE01664 Novel human protease #3 | 1013 | 1.8e−174 |
| patp: AAR97618 Human carboxypeptidase A1 | 1682 | 7.2e−173 |
| patp: AAY28915 Human regulatory protein | 1682 | 7.2e−173 |
| HRGP-1 | | |

In a search of sequence databases, it was found, for example, that the nucleic acid sequence of this invention has 1017 of 1264 bases (80%) identical to a gb:GENBANK-ID:HSPCBXA1|acc:X67318.1 mRNA from Homo sapiens (H.sapiens mRNA for procarboxypeptidase A1). The full amino acid sequence of the protein of the invention was found to have 315 of 419 amino acid residues (75%) identical to, and 357 of 419 amino acid residues (85%) similar to, the 419 amino acid residue ptnr:SWISSNEW-ACC:P15085 protein from Homo sapiens (Human) (CARBOXYPEPTIDASE A1 PRECURSOR (EC 3.4.17.1)). NOV97 also has homology to the other proteins shown in the BLASTP data in Table 97N.

TABLE 97N
NOV97 BLASTP results

| Gene Index/ | | Length | Identity | Positive | |
|---|---|---|---|---|---|
| Identifier | Protein/Organism | (aa) | (%) | (%) | Expect |
| gi\|17939400\|ref\| | carboxypeptidase A5 | 436 | 354/418 | 382/418 | 0.0 |
| NP_525124.1\| | [Homo sapiens] | | (84) | (90) | |
| (NM_080385) | | | | | |
| gi\|4502997\|ref\| | pancreatic | 419 | 315/419 | 357/419 | 0.0 |
| NP_001859.1\| | carboxypeptidase A1 | | (75) | (85) | |
| (NM_001868) | precursor; | | | | |
| | Carboxypeptidase A | | | | |
| | [Homo sapiens] | | | | |
| gi\|13528975\|gb\| | carboxypeptidase A1 | 419 | 314/419 | 356/419 | 0.0 |
| AAH05279.1\|AAH05279 | (pancreatic) | | (74) | (84) | |
| (BC005279) | [Homo sapiens] | | | | |
| gi\|4336196\|gb\|AAD17690.1\| | carboxypeptidase A1 | 419 | 295/419 | 345/419 | e−177 |
| (AF076222) | precursor [Sus scrofa] | | (68) | (81) | |
| gi\|115878\|sp\|P00730\| | CARBOXYPEPTIDASE A | 419 | 289/419 | 345/419 | e−176 |
| CBPA_BOVIN | PRECURSOR | | (68) | (81) | |

This BLASTP data is displayed graphically in the ClustalW in Table 97O. A multiple sequence alignment is given, with the NOV97a-f proteins being shown on lines 1-6 in a ClustalW analysis comparing the protein of the invention with the related protein sequences shown in Table 97N.

[image]

[image]

[image]

Table 97P lists the domain description from DOMAIN analysis results against NOV97. This indicates that the NOV97 sequence has properties similar to those of other proteins known to contain this domain.

TABLE 97P
Domain Analysis of NOV97

gnl|Smart|smart00631, Zn_pept, Zn_pept domain SEQ ID NO:887
CD-Length = 286 residues, 100.0% aligned
Score = 301 bits (770), Expect = 6e−83

| | | | |
|---|---|---|---|
| NOV97: | 122 | YHTLEEVYSWIDNFVMEHSDIVSKIQIONSFENOSILVLKFSTGGSR--HPAIWIDTGIH | 179 |
| | | YH= EE= =W= = D=V = IG S E = I VLK S G R PA=WID GIH | |
| Sbjct: | 1 | YHSYEEIEAWLKKLAARYPDLVRLVSIGKSVEGRPIWVLKISNGPGRDGKPAVWIDAGIH | 60 |
| NOV97: | 180 | SREWITHATGIWTANKIVSDYGKDRVLTDILNAMDIFIELVTNPDGFAFTHSMNRLWRKN | 239 |
| | | =REWI AT == N=== =YG D =T =L= D =I V NPDG= =TH= =RLWRKN | |
| Sbjct: | 61 | AREWIGPATALYLINQLLENYGSDPRVTKLLDKTDWYIVPVLNPDGYEYTHTSDRLWRKN | 120 |
| NOV97: | 240 | KSIRPGIFCIGVDLNRWKSGFGGNGSNSNPCSETYHGPSPQSEPEVAAIVNFITAHGNF | 299 |
| | | =S G C GVDLNRN= =G G==SNPCSETY GPSP SEPE A= =F= == | |
| Sbjct: | 121 | RSPNSGSNCRGVDLNRNFPFHWGETGASSNPCSETYAGPSPFSEPETKAVRDFLRSNRKI | 180 |
| NOV97: | 300 | KALISIHSYSQMLMYPYGRLLEPV-SNQRELYDLAKDAVEALYKVHG-IEYIFGSISTTL | 357 |
| | | K I =HSYSQ===YPYG = N =L ==AK =AL VHG Y =G = L | |
| Sbjct: | 181 | KLYIDLHSYSQLILYPYGYTKNDLPPNVEDLPEVAKALADALASVHGGTRYTYGISNGAL | |
| NOV97: | 358 | YVASGITVDWAYD-SGIKYAFSFELRDTGQYGFLLPATQIIPTAQE 402 | |
| | | Y ASG = DWAY G= ==== ELRD G=YGFLLP =QIIPT E | |
| Sbjct: | 241 | YPASGGSDDWAYGTLGVPFSYTLELRDKGRYGFLLPPSQIIPTGWE 286 | |

Carboxypeptidase A (EC 3.4.2.1) is a pancreatic exopeptidase. Three different forms of human pancreatic procarboxypeptidase A have been isolated. The A1 and A2 (600688) forms are monomeric proteins with different biochemical properties. Honey et al. (1984, 1986) found that an 8.6-kb human DNA fragment (detected by means of a rat cDNA probe for CPA) cosegregated with chromosome 7. The assignment was narrowed by demonstration of absence of the human DNA fragment in cells with a deletion of 7q22-qter. By studying mouse-hamster hybrid cells, Honey et al. (1986) assigned the CPA gene to mouse chromosome 6. Trypsin (276000) is also on human 7q22-qter and on mouse 6. Stewart et al. (1990) concluded from multipoint linkage analysis with established chromosome 7 markers that the most likely location of carboxypeptidase is 7q31-qter. It lies distal to cystic fibrosis at a distance of approximately 12 cM.

NOV97 is predicted to be expressed in at least the following tissues: pancreas. This information was derived by determining the tissue sources of the sequences that were included in the invention including but not limited to SeqCalling sources, public EST sources, literature sources, and/or RACE sources. Further expression data for NOV97 is provided in Example 2.

The NOV97 nucleic acids and proteins are useful in potential therapeutic applications implicated in various pathological disorders described further herein, for example, Von Hippel-Lindau (VHL) syndrome, Alzheimer's disease, stroke, tuberous sclerosis, hypercalceimia, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, neuroprotection, hemophilia, hypercoagulation, idiopathic thrombocytopenic purpura, immunodeficiencies, hemophilia, hypercoagulation, immunodeficiencies, graft vesus host disease as well as other diseases, disorders and conditions. NOV97 nucleic acids encoding the carboxypeptidase-like protein of the invention, or fragments thereof, may further be useful in diagnostic applications, wherein the presence or amount of the nucleic acid or the protein are to be assessed.

The novel nucleic acid of the invention encoding a carboxypeptidase A1-like protein includes the nucleic acid whose sequence is provided in Table 97A, 97C, 97E, 97G, 971, or 97K or a fragment thereof. The invention also includes a mutant or variant nucleic acid any of whose bases may be changed from the corresponding base shown in Table 97A, 97C, 97E, 97G, 97I, or 97K while still encoding a protein that maintains its carboxypeptidase A1-like activities and physiological functions, or a fragment of such a nucleic acid. The invention further includes nucleic acids whose sequences are complementary to the sequences of Table 97A, 97C, 97E, 97G, 97I, or 97K, including nucleic acid fragments that are complementary to any of the nucleic acids just described.

The invention additionally includes nucleic acids or nucleic acid fragments, or complements thereto, whose structures include chemical modifications. Such modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 20% of the bases may be so changed.

The novel protein of the invention includes the carboxypeptidase A1-like protein whose sequence is provided in Table 97B, 97D, 97F, 97H, 97J, or 97L. The invention also includes a mutant or variant protein any of whose residues may be changed from the corresponding residue shown in Table 97B, 97D, 97F, 97H, 97J, or 97L while still encoding a protein that maintains its carboxypeptidase A1-like activities and physiological functions, or a functional fragment thereof. In the mutant or variant protein, up to about 25% of the amino acid residues may be so changed.

These materials are further useful in the generation of antibodies that bind immunospecifically to the substances of the invention for use in therapeutic or diagnostic methods. These antibodies may be generated according to methods known in the art, using prediction from hydrophobicity charts, as described in the “Anti-NOVX Antibodies” section below.

NOV98

The disclosed NOV98 (alternatively referred to herein as CG56939-01) includes the 5583 nucleotide sequence (SEQ ID NO:319) shown in Table 98A. A NOV98 ORF begins with a Kozak consensus ATG initiation codon at nucleotides 1-3 and ends with a TGA codon at nucleotides 4630-4632. The disclosed NOV98 maps to human chromosome 1.

TABLE 98A
NOV98 Nucleotide Sequence

| | |
|---|---|
| GCCGCGTGCGTCCTGCCCCGAGCCGGCGCGACATGCCCGCAGCCCGCGCTGGAGCGGCGC | (SEQ ID NO:319) |
| GAGGAGGAGGCGAACGTGGTGCTCACCGGGACGGTGGACGAGATCCTCAACGTGGACCCG | |
| GTGCAGCACACGTACTCCTGCAAGGTTCGGGTCTGGCGGTACTTGAAGGGCAAAGACCTG | |
| GTGGCCCGGGAGAGCCTGCTGGACGGCGGCAACAAGGTGGTGATCAGCGGCTTTGGAGAC | |
| CCCCTCATCTGTGACAACCAGGTGTCCACTGGGGACACCAGGATCTTCTTTGTGAACCCT | |
| GCACCCCCATACCTGTGGCCAGCCCACAAGAACGAGCTGATGCTCAACTCCAGCCTCATG | |
| CGGATCACCCTGCGGAACCTGGAGGAGGTGGAGTTCTCTGTGGAAGATAAACCCGGGACC | |
| CACTTCACTCCAGTGCCTCCGACGCCTCCTGATGCGTGCCGGGGAATGCTGTGCGGCTTC | |
| GGCGCCGTGTGCGAGCCCAACGCGGAGGCGCCGGGCCGGGCGTCCTGCGTCTGCAAGAAG | |
| AGCCCGTCCCCCAGCGTGGTGGCGCCTGTGTGTGGGTCGGACGCCTCCACCTACAGCAAC | |
| GAATGCGAGCTGCAGCGGGCGCAGTGCAGCCAGCAGCGCCGCATCCGCCTGCTCAGCCGC | |
| GGGCCGTGCGGCTCGCGGGACCCCTCCTCCAACGTGACCTGCAGCTTCGGCAGCACCTGT | |
| GCGCGCTCGGCCGACGCGCTGACGGCCTCGTGCCTGTGCCCCGCGACCTGCCGTGGCGCC | |
| CCCGAGGGGACCGTCTGCGGCAGCGACGGCGCCGACTACCCCGGCGAGTGCCAGCTCCTG | |
| CGCCGCGCCTGCGCCCGCCAGGAGAATGTCTTCAAGAAGTTCGACGGCCCTTGTGACCCC | |
| TGTCAGGGCGCCCTCCCTGACCCGAGCCGCAGCTGCCGTGTGAACCCGCGCACGCCGCGC | |
| CCTGAGATGCGCCTACGGCCCGAGAGCTGCCCTGCCCGGCAGGCGCCAGTGTGTGGGGAC | |
| GACGGAGTCACCTACGAAAACGACTGTGTCATGGGCCGATCGGGGGCCGCCCGGGGTCTC | |
| CTCCTGCAGAAAGTGCGCTCCGGCCAGTGCCAGGGTCGAGACCAGTGCCCGGAGCCCTGC | |
| CGGTTCAATGCCGTGTGCCTGTCCCGCCGTGGCCGTCCCCGCTGCTCCTGCGACCGCGTC | |
| ACCTGTGACGCGGCCTACAGGCCCGTGTGTGCCCAGGACGGGCGCACGTATGACAGTGAT | |
| TGCTGGCGGCAGCAGGCTGAGTGCCGGCAGCAGCGTGCCATCCCCAGCAAGCACCAGGGC | |
| CCGTGTGACCAGGCCCCGTCCCCATGCCTCGGGGTGCAGTGTGCATTTGGGGCGACGTGT | |
| GCTGTGAAGAACGGGCAGGCAGCGTGTGAATGCCTGCAGGCGTGCTCGAGCCTCTACGAT | |
| CCTGTGTGCGGCAGCGACGGCGTCACATACGGCAGCCCGTGCCAGCTGGAGGCCACGGCC | |
| TGTACCCTCGGGCGGGAGATCCAGGTGGCGCGCAAAGGACCCTGTGACCGCTGCGGGCAG | |
| TGCCGCTTTGGAGCCCTGTGCGAGGCCGAGACCGCGCGCTGCGTGTGCCCCTCTGAATCC | |
| CTGGCTTTGGCCCAGCCCGTGTGTGGCTCCGACGGGCACACGTACCCCAGCGAGTGCATG | |
| CTGCACGTGCACGCCTGCACACACCAGATCAGCCTGCACGTGGCCTCAGCTGGACCCTGC | |
| GAGACCTGTGGAGATGCCGTGTGTGCTTTTCGGGCTGTGTGCTCCGCAGGGCAGTGTGTG | |
| TGTCCCCGGTGTGAGCACCCCCCGCCCGGCCCCGTGTGTGGCAGCGACGGTGTCACCTAC | |
| GGCAGTGCCTGCGAGCTACGGGAAGCCGCCTGCCTCCAGCAGACACAGATCGAGGAGGCC | |
| CGGGCAGGGCCGTGCGAGCAGGCCGAGTGCGGTTCCGGAGGCTCTGGCTCTGGGGAGGAC | |
| GGTGACTGTGAGCAGGAGCTGTGCCGGCAGCGCGGTGGCATCTGGGACGAGGACTCGGAG | |
| GACGGGCCGTGTGTCTGTGACTTCAGCTGCCAGAGTGTCCCAGGCAGCCCGGTGTGCGGC | |
| TCAGATGGGGTCACCTACAGCACCGAGTGTGAGCTGAAGAAGGCCAGGTGTCAGTCACAG | |
| CGAGGGCTCTACGTAGCGGCCCAGGGAGCCTGCCGAGGCCCCACCTTCGCCCCGCTGCCG | |
| CCTGTGGCCCCCTTACACTGTGCCCAGACGCCCTACGGCTGCTGCCAGCACAATATCACC | |
| GCAGCCCGGGGCGTGGGCCTGGCTGGCTGCCCCAGTGCCTGCCAGTGCAACCCCCATGGC | |
| TCTTACGGCGGCACCTGTGACCCAGCCACAGGCCAGTGCTCCTGCCGCCCAGGTGTGGGG | |
| GGCCTCAGGTGTGACCGCTGTGAGCCTGGCTTCTGGAACTTTCGAGGCATCGTCACCGAT | |
| GGCCGGAGTGGCTGTACACCCTGCAGCTGTGATCCCCAAGGCGCCGTGCGGGATGACTGT | |
| GAGCAGATGACGGGGCTGTGCTCGTGTAAGCCCGGGGTGGCTGGACCCAAGTGTGGGCAG | |
| TGTCCAGACGGCCGTGCCCTGGGCCCCGCCGGCTGTGAAGCTGACGCTTCTGCGCCTGCG | |
| ACCTGTGCGGAGATGCGCTGTGAGTTCGGTGCGCGGTGCGTGGAGGAGTCTGGCTCAGCC | |
| CACTGTGTCTGCCCGATGCTCACCTGTCCAGAGGCCAACGCTACCAAGGTCTGTGGGTCA | |
| GATGGAGTCACATACGGCAACGAGTGTCAGCTGAAGACCATCGCCTGCCGACGGTGTCAC | |
| CTACGCCAGGGCCTGCAAATCTCTATCCAGAGCCTGGGCCCGTGCCAGGAGGCTGTTGCT | |
| CCCAGCACTCACCCGACATCTGCCTCCGTGACTGTGACCACCCCAGGGCTCCTCCTGAGC | |
| CAGGCACTGCCGGCCCCCCCCGGCGCCCTCCCCCTGGCTCCCAGCAGTACCGCACACAGC | |
| CAGACCACCCCTCCGCCCTCATCGCGACCTCGGACCACTGCCAGCGTCCCCAGCACCACC | |
| GTGTGGCCCGTGCTGACGGTGCCCCCCACGCCACCCTCCCCTGCACCCAGCCTGGTGGCG | |
| TCCGCCTTTGGTGAATCTGGCAGCACTGATGGAAGCAGCGATGAGGAACTGAGCGGGGAC | |
| CAGGAGGCCAGTGGGGGTGGCTCTGGGGGGCCCGAGCCCTTGGAGGGCAGCAGCGTGGCC | |
| ACCCCTGGGCCACCTGTCGAGAGGGCTTCCTGCTACAACCCCTGCCATGGGGCGGCGCCC | |
| TGCCGTGTGCTGCCCGAGGGTGGTGCTCAGTGCGAGTGCCCCCTGGGGCGTGAGGGCACC | |
| TTCTGCCAGACAGCCTCGGGGCAGGACGGCTCTGGGCCCTTCCTGGCTGACTTCAACGGC | |
| TTCTCCCACCTGGAGCTGAGAGGCCTGCACACCTTTGCACGGGACCTGGGGGAGAAGATG | |
| GCGCTGGAGGTCGTGTTCCTGGCACGAGGCCCCAGCGGCCTCCTGCTCTACAACGGGCAG | |
| AAGACGGACGGCAAGGGGGACTTCGTGTCGCTGCCACTGCGGGACCCCCGCCTGGAGTTC | |
| CGCTACGACCTGGGCAAGGCGGCAGCGGTCATCAGCAGCAGGGAGCCAGTCACCCTGGGA | |
| GCCTGGACCAGGGTCTCACTGGAGCGAAACGGCCGCAAGGGTGCCCTGCGTGTGGGCGAC | |
| GGCCCCCGTGTGTTGGGGGAGTCCCCGGTTCCGCACACCGTCCTCAACCTGAAGGAGCCG | |
| CTCTACGTAGGGGGCGCTCCCGACTTCAGCAAGCTGCCCCGTGCTGCTGCCGTGTCCTCT | |
| GGCTTCGACGGTGCCATCCAGCTGGTCTCCCTCGGACGCCGCCAGCTGCTGACCCCGGAG | |
| CACGTGCTGCGGCAGGTGGACGTCACGTCCTTTGCAGGTCACCCCTGCACCCGGGCCTCA | |
| GGCCACCCCTGCCTCAATGGGGCCTCCTGCGTCCCGAGCGAGGCTGCCTATGTGTGCCTG | |
| TGTCCCGGGGGATTCTCAGGACCGCACTGCGAGAAGGGGCTGGTGCAGAAGTCAGCGGGG | |
| GACGTGGATACCTTGGCCTTTGACGGGCGGACCTTTGTCGAGTACCTCAACGCTGTGACC | |
| GAGAGCGAGAAGGCACTGCAGAGCAACCACTTTGAACTGAGCCTGCGCACTGAGGCCACG | |
| CAGGGGCTGGTGCTCTGGAGTGGCAAGGCCACGGAGCGGGCAGACTATGTGGCACTGGCC | |
| ATTGTGGACGGGCACCTGCAACTGAGCTACAACCTGGGCTCCCAGCCCGTGGTGCTGCGT | |
| TCCACCGTGCCCGTCAACACCAACCGCTGGTTGCGGGTCGTGGCACATAGGGAGCAGAGG | |
| GAAGGTTCCCTGCAGGTGGGCAATGAGGCCCCTGTGACCGGCTCCTCCCCGCTGGGCGCC | |
| ACGCAGCTCGACACTGATGGAGCCCTGTGGCTTGGGGGCCTGCCGGAGCTGCCCGTGGGC | |
| CCAGCACTGCCCAAGGCCTACGGCACAGGCTTTGTGGGCTGCTTGCGGGATGTGGTGGTG | |
| GCCCGGCACCCGCTGCACCTGCTCGAGGACGCCGTCACCAAGCCAGAGCTGCGGCCCTGC | |
| CCCACCCCATGAGCTGGCACCAGAGCCCCGCGCCCGCTGTAATTATTTTCTATTTTTGTA | |
| AACTTGTTCCTTTTTGATATGATTTTCTTGCCTGAGTGTTGGCCGGAGGGACTGCTGGCC | |
| CGGCCTCCCTTCCGTCCAGGCAGCCGTGCTGCAGACAGACCTAGTGCTGAGGGATGGACA | |
| GGCGAGGTGGCAGCGTGGAGGGCTCGGCGTGGATGGCAGCCTCAGGACACACACCCCTGC | |
| CTCAAGGTGCTGAGCCCCCGCCTTGCACTGCGCCTGCCCCACGGTGTCCCCGCCGGGAAG | |
| CAGCCCCGGCTCCTGAATCACCCTCGCTCCGTCAGGCGGGACTCGTGTCCCAAAAAGGAA | |
| GGGGCTGCTGAGGTCTGATGGGGCCCTTCCTCCGGGTGACCCCACAGGGCCTTTCCAAGC | |
| CCCTATTTGACCTGCTCCTTCCTGTGTGTGCTCTGGACCCTGCCTCGGCCTCCTGCGCCA | |
| ATACTGTGACTTCCAAACAATGTTACTGCTGCGCACAGCTCTGCGTTGCTCCCGTGCTGC | |
| CTGCGCCAGCCCCAGGCTGCTGAGGAGCACAGGCCAGACCAGGGCCGATCTGGGTGTCCT | |
| GACCCTCAGCTGGCCCTGCCCAGCCACCCTGGACATGACCGTATCCCTCTGCCACACCCC | |
| AGGCCCTGCGAGGGGCTATCGAGAGGAGCTCACTGTGGGATGGGGTTGACCTCTGCCGCC | |
| TGCCTCGGTATCTGGGCCTGGCCATGGCTGTGTTCTTCATGTGTTGATTTTATTTGACCC | |
| CTGGAGTGGTGGGTCTCATCTTTCCCATCTCGCCTGAGAGCGGCTGAGGCCTGCCTCACT | |
| GCAAATCCTCCCCACAGCGTCAGTGAAAGTCGTCCTTGTCTCAGAATGACCAGGGGCCAG | |
| CCAGTGTCTGACCAAGGTCAAGGGGCAGGTGCAGAGGTGGCAGGGATGGCTCCGAAGCCA | |
| GAA | |

A NOV98 polypeptide (SEQ ID NO:320) encoded by SEQ ID NO:319 is 1543 amino acids in length and is presented using the one-letter amino acid code in Table 98B. The Psort profile for NOV98 predicts that this sequence has no signal peptide and is likely to be localized to the cytoplasm with a certainty of 0.4500. In alternative embodiments, a NOV98 polypeptide is located to microbodies with a certainty of 0.3000.

TABLE 98B
NOV98 Polypeptide Sequence

| | |
|---|---|
| AACVLPGAGGTCPERALERREEEANVVLTGTVEEILNVDPVQHTYSCKVRVWRYLKGKDL | (SEQ ID NO:320) |
| VARESLLDGGNKVVISGFGDPLICDNQVSTGDTRIFFVNPAPPYLWPAHKNELMLNSSLM | |
| RITLRNLEEVEFCVEDKPGTHFTPVPPTPPDACRGMLCGFGAVCEPNAEGPGRASCVCKK | |
| SPCPSVVAPVCGSDASTYSNECELQRAQCSQQRRIRLLSRGPCGSRDPCSNVTCSFGSTC | |
| ARSADGLTASCLCPATCRGAPEGTVCGSDGADYPGECQLLRRACARQENVFKKFDGPCDP | |
| CQGALPDPSRSCRVNPRTRRPEMRLRPESCPARQAPVCGDDGVTYENDCVNGRSGAARCL | |
| LLQKVRSGQCQGRDQCPEPCRFNAVCLSRRGRPRCSCDRVTCDGAYRPVCAQDGRTYDSD | |
| CWRQQAECRQQRAIPSKHQCPCDQAPSPCLGVQCAFGATCAVKNGQAACECLQACSSLYD | |
| PVCGSDGVTYGSACELEATACTLCREIQVARKGPCDRCGQCRFGALCEAETGRCVCPSEC | |
| VALAQPVCGSDGHTYPSECMLHVHACTHQISLHVASAGPCETCGDAVCAFGAVCSAGQCV | |
| CPRCEHPPPCPVCGSDGVTYGSACELREAACLQQTQIEEARAGPCEQAECGSGGSGSGED | |
| GDCEQELCRQRGGIWDEDSEDGPCVCDFSCQSVPGSPVCGSDGVTYSTECELKKARCESQ | |
| RGLYVAAQGACRGPTFAPLPPVAPLHCAQTPYGCCQDNITAARGVGLAGCPSACQCNPHG | |
| SYGGTCDPATGQCSCRPGVGGLRCDRCEPGFWNFRGIVTDGRSGCTPCSCDPQGAVRDDC | |
| EQMTGLCSCKPGVAGPKCGQCPDGRALGPAGCEADASAPATCAEMRCEFGARCVEESGSA | |
| HCVCPMLTCPEANATRVCGSDGVTYGNECQLKTTACRRCHLRQGLQISIQSLGPCQEAVA | |
| PSTHPTSASVTVTTPGLLLSQALPAPPGALPLAPSSTAHSQTTPPPSSRPRTTASVPRTT | |
| VWPVLTVPPTAPSPAPSLVASAFGESGSTDGSSDEELSGDQEASGGGSGGPEPLEGSSVA | |
| TPGPPVERASCYNPCHGAAPCRVLPEGGAQCECPLGREGTFCQTASGQDGSGPFLADFNG | |
| FSHLSLRCLHTFARDLGEKMALEVVFLARGPSGLLLYNGQKTDGKGDFVSLALRDRRLEF | |
| RYDLGKGAAVIRSREPVTLGAWTRVSLERNGRKGALRVGDGPRVLCESPVPHTVLNLKEP | |
| LYVGGAPDFSKLARAAAVSSGFDGAIQLVSLGGRQLLTPEHVLRQVDVTSFAGHPCTRAS | |
| GHPCLNGASCVPREAAYVCLCPGGFSGPHCSKGLVEKSAGDVDTLAFDGRTFVEYLNAVT | |
| ESEKALQSNHFELSLRTEATQGLVLWSGKATERADYVALAIVDGHLQLSYNLGSQPVVLR | |
| STVPVNTNRWLRVVAHREQREGSLQVGNEAPVTGSSPLGATQLDTDGALWLGGLPELPVG | |
| PALPKAYGTGFVGCLRDVVVGRHPLHLLEDAVTKPELRPCPTP | |

A BLAST analysis of NOV98 was run against the proprietary PatP GENESEQ Protein Patent database. It was found, for example, that the amino acid sequence of NOV98 had high homology to other proteins as shown in Table 98C.

TABLE 98C
BLASTX results from PatP database for NOV98

| | | Smallest |
|---|---|---|
| | | Sum |
| | High | Probability |
| Sequences producing High-scoring Segment Pairs: | Score | P(N) |
| patp: AAW26609 Human agrin - Homo | 2349 | 2.2e−246 |
| sapiens, 492 aa. | | |
| patp: AAB93754 Human protein sequence | 2179 | 1.5e−225 |
| patp: AAY73993 Human prostate | 2177 | 2.5e−225 |
| tumor EST fragment | | |
| patp: AAB31889 Amino acid sequence of | 380 | 1.2e−62 |
| a human protein | | |
| patp: AAU16938 Human novel secreted protein | 551 | 1.2e−51 |

In a search of sequence databases, it was found, for example, that the nucleic acid sequence of this invention has 3158 of 3416 bases (92%) identical to a gb:GENBANK-ID:AF016903|acc:AF016903.1 mRNA from Homo sapiens (agrin precursor mRNA). The full amino acid sequence of the protein of the invention was found to have 1092 of 1114 amino acid residues (98%) identical to, and 1093 of 1114 amino acid residues (98%) similar to, the 2026 amino acid residue ptnr:SPTREMBL-ACC:O00468 protein from Homo sapiens (Human) (AGRIN PRECURSOR). NOV98 also has homology to the other proteins shown in the BLASTP data in Table 98D.

TABLE 98D
NOV98 BLASTP results

| Gene Index/ | | Length | Identity | Positive | |
|---|---|---|---|---|---|
| Identifier | Protein/Organism | (aa) | (%) | (%) | Expect |
| gi\|2988422\|gb\| | agrin precursor | 2026 | 1088/1097 | 1088/1097 | 0.0 |
| AAC39776.1\| | [Homo sapiens] | | (99) | (99) | |
| (AF016903) | | | | | |
| gi\|399021\|sp\| | Agrin precursor | 1959 | 772/958 | 825/958 | 0.0 |
| P25304\|AGRI_RAT | | | (80) | (85) | |
| gi\|202800\|gb\| | agrin [Rattus | 1940 | 772/958 | 825/958 | 0.0 |
| AAA40703.1\| | norvegicus] | | (80) | (83) | |
| (M64780) | | | | | |
| gi\|202799\|gb\| | agrin [Rattus | 1937 | 769/958 | 822/958 | 0.0 |
| AAA40702.1 | norvegicus] | | (80) | (85) | 0.0 |
| (M64780) | | | | | |
| gi\|399020\|sp\| | Agrin precursor | 1169 | 637/1017 | 758/1017 | 0.0 |
| P31696\|AGRI_CHICK | | | (62) | (73) | |

This BLASTP data is displayed graphically in the ClustalW in Table 98E. A multiple sequence alignment is given, with the NOV98 protein being shown on line 1 in a ClustalW analysis comparing the protein of the invention with the related protein sequences shown in Table 98D.

[image]

[image]

[image]

[image]

[image]

[image]

[image]

Table 98F lists the domain description from DOMAIN analysis results against NOV98. This indicates that the NOV98 sequence has properties similar to those of other proteins known to contain this domain.

TABLE 98F
Domain Analysis of NOV98

gnl|Pfam|pfam00054, laminin_C, Laminin G domain SEQ ID NO:888
CD-Length = 134 residues, 99.3% aligned
Score = 152 bits (385), Expect = 1e−37

| | | | |
|---|---|---|---|
| NOV98: | 1396 | RTEATQGLVLWSGKATERDYVALAIVDGHLQLSYNLGSQPVVLRSTVPVNTNRWLRVVA | 1455 |
| | | RT GL+L+ G T+R D++AL + DG L++SY+LGS P V+RS +N +W RV | |
| Sbjct: | 2 | RTTEPSGLLLYGGTNTDR-DFLALELRDGRLEVSYDLGSGPAVVRSGDRLNDGKWHRVEL | 60 |
| NOV98: | 1456 | HREQREGSLQVGNEAPVTGSSPLGATQ----LDTDGALWLCGLPELPVGPALPKAYGTGF | 1511 |
| | | R R+G+L V E V G SP G LD D L++GGLPE L A T F | |
| Sbjct: | 61 | ERNGRKGTLSVDGEESVDGESPSGPDVPHENLDLDTPLYVGGLPE-LSVKRLAAISTSF | 119 |
| NOV98: | 1512 | VGCLRDVVVGRHPLH | 1526 |
| | | GC+RDV+V PL | |
| Sbjct: | 120 | KGCIRDVIVNGKPLD | 134 |

Synapses are essential relay stations for the transmission of information between neurones and other cells. An ordered and tightly regulated formation of these structures is crucial for the functioning of the nervous system. The synapse is also involved in perception, learning and memory. Understanding the sequence of steps that is involved in establishing synapses during development might also help to understand mechanisms that cause changes in synapses during learning and memory.

For practical reasons, most of the current knowledge of synapse development is derived from studies of the vertebrate neuromuscular junction. Upon arrival of a motor axon at the muscle fiber, signals released from its growth cone initiate the formation of a synapse. This process consists of two stages: arrest of axon growth at the target area and differentiation of pre- and postsynaptic cells at the site of nerve-muscle contact.

Studies of regenerating neuromuscular junctions in vertebrates have revealed that important signals for the formation of this synapse are located in the synaptic basal lamina, and attempts to identify these signals have led to the isolation of agrin and other components. The induction of the intensively studied synapse between nerve and muscle is initiated by the binding of neuron-specific isoforms of the basal membrane protein agrin to receptors on the surface of myotubes. Agrin activates a receptor complex that includes the muscle-specific kinase and most likely additional, yet to be identified, components. Receptor activation leads to the aggregation of acetylcholine receptors (AChR) and other proteins of the postsynaptic apparatus. This activation process has unique features which distinguish it from other receptor tyrosine kinases. In particular, the autophosphorylation of the kinase domain, which usually induces the recruitment of adaptor and signalling molecules, is not sufficient for AChR aggregation. Apparently, interactions of the extracellular domain with unknown components are also required for this process.

Agrin binds to a second protein complex on the muscle surface known as the dystrophin-associated glycoprotein complex. This binding forms one end of a molecular link between the extracellular matrix and the cytoskeleton.

While many components of the machinery triggering postsynaptic differentiation have now been identified, the picture of the molecular pathway causing the redistribution of synaptic proteins is still incomplete. Recent advances implicate proteins such as dystroglycan, MuSK, and rapsyn in the transduction of agrin signals. Additional functions of agrin have been discovered, including the upregulation of gene transcription in myonuclei and the control of presynaptic differentiation.

Agrin therefore appears to play a unique role in controlling synaptic differentiation on both sides of the neuromuscular junction.

NOV98 is predicted to be expressed in at least the following tissues: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea and uterus, bone, cerebral medulla/cerebral white matter, cervix, colon, epidermis, foreskin, hair follicles, liver, lung, lymphoid tissue, ovary, parathyroid gland, parietal lobe, retina, skin, vein, whole organism. This information was derived by determining the tissue sources of the sequences that were included in the invention including but not limited to SeqCalling sources, public EST sources, literature sources, and/or RACE sources. Further expression data for NOV98 is provided in Example 2.

The NOV98 nucleic acids and proteins are useful in potential therapeutic applications implicated in various pathological disorders described further herein, for example, Von Hippel-Lindau (VHL) syndrome, Alzheimer's disease, stroke, tuberous sclerosis, hypercalceimia, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, neuroprotection, hemophilia, hypercoagulation, idiopathic thrombocytopenic purpura, immunodeficiencies, hemophilia, hypercoagulation, immunodeficiencies, graft vesus host disease as well as other diseases, disorders and conditions. NOV98 nucleic acids encoding the agrin-like protein of the invention, or fragments thereof, may further be useful in diagnostic applications, wherein the presence or amount of the nucleic acid or the protein are to be assessed.

The novel nucleic acid of the invention encoding a agrin-like protein includes the nucleic acid whose sequence is provided in Table 98B, or a fragment thereof. The invention also includes a mutant or variant nucleic acid any of whose bases may be changed from the corresponding base shown in Table 98B while still encoding a protein that maintains its agrin-like activities and physiological functions, or a fragment of such a nucleic acid. The invention further includes nucleic acids whose sequences are complementary to the sequence of Table 98B, including nucleic acid fragments that are complementary to any of the nucleic acids just described.

The invention additionally includes nucleic acids or nucleic acid fragments, or complements thereto, whose structures include chemical modifications. Such modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 8% of the bases may be so changed.

The novel protein of the invention includes the agrin-like protein whose sequence is provided in Table 98B. The invention also includes a mutant or variant protein any of whose residues may be changed from the corresponding residue shown in Table 98B while still encoding a protein that maintains its agrin-like activities and physiological functions, or a functional fragment thereof. In the mutant or variant protein, up to about 2% of the amino acid residues may be so changed.

These materials are further useful in the generation of antibodies that bind immunospecifically to the substances of the invention for use in therapeutic or diagnostic methods. These antibodies may be generated according to methods known in the art, using prediction from hydrophobicity charts, as described in the “Anti-NOVX Antibodies” section below.

NOV99

The disclosed NOV99 (alternatively referred to herein as CG57010-01) includes the 1513 nucleotide sequence (SEQ ID NO:321) shown in Table 99A. A NOV99 ORF begins with a Kozak consensus ATG initiation codon at nucleotides 396-398 and ends with a TGA codon at nucleotides 1410-1412. The disclosed NOV99 maps to human chromosome 14q32.33.

TABLE 99A
NOV99 Nucleotide Sequence

| | |
|---|---|
| GGAGCCCCCGCCCTGGGATTCCCAGGTGTTTTCATTTGGTGATCAGCACTGAACACAGAA | (SEQ ID NO:321) |
| GAGTCATGACGGAGTTTGGGCTGAGCTGGGTTTTCCTTGTTGCTATTTTTAAAGGTGTCC | |
| AGTGTGAGGTGCAGCTGGTGGAGTCTGGGGGAGACTTGGTCCAGCCTGGGGGGTCCCTGA | |
| GACTCTCCTGTGCAGCCTCTGGATTCACCTTCAGTAGTTATGCTATGCACTGGGTCCGCC | |
| AGGCTCCAGGGAAGGGACTGAAATATGTTTCAGGTATTAGTAGTAATGGGCGTAGAACAT | |
| ATTATGCAAATTCTGTGAAGGGCAGATTCACCATCTCCAGAGACAATTCCAAGAACACGT | |
| TGTATCTTCAAATGGGCAGCCTCAGAGCTGAGGACATGGCTGTGTATTACTGTGTGTCCG | |
| GCGGAATCTATGATAGTAGTGGTCCCTTTGACTACTGGGGCCAGGGAACCCTGGTCACCG | |
| TCTCCTCAGCATCCCCGACCAGCCCCAAGGTCTTCCCGCTGAGCCTCTGCAGCACCCAGC | |
| CAGATGGGAACGTGGTCATCGCCTGCCTGGTCCAGGGCTTCTTCCCCCAGGAGCCACTCA | |
| GTGTGACCTGGAGCGAAAGCGGACAGGGCGTGACCGCCAGAAACTTCCCACCCAGCCAGG | |
| ATGCCTCCGGGGACCTGTACACCACGAGCAGCCAGCTGACCCTGCCGGCCACACAGTGCC | |
| TAGCCGGCAAGTCCGTGACATGCCACGTGAAGCACTACACGAATCCCAGCCAGGATGTGA | |
| CTGTGCCCTGCCCAGTTCCCTCAACTCCACCTACCCCATCTCCCTCAACTCCACCTACCC | |
| CATCTCCCTCATGCTGCCACCCCCGACTGTCACTGCACCGACCGGCCCTCGAGGACCTGC | |
| TCTTAGGTTCAGAAGCGAACCTCACGTGCACACTGACCGGCCTGAGAGATGCCTCAGCTG | |
| TCACCTTCACCTGGACGCCCTCAAGTGGGAAGAGCGCTGTTCAAGGACCACCTGAGCGTG | |
| ACCTCTGTGGCTGCTACAGCGTGTCCAGTGTCCTGCCTGCCTGTGCCCAGCCATGGAACC | |
| ATGGGGAGACCTTCACCTGCACTGCTGCCCACCCCGAGTTGAAGACCCCACTAGTTCGCT | |
| GGCTGCAGGGGTCACAGGAGCTGCCCCGCGAGAAGTACCTGACTTGGGCATCCCGGCAGG | |
| AGCCCAGCCAGGGCACCACCACCTTCGCTGTGACCAGCATACTGCGCGTGGCAGCCGAGG | |
| ACTGGAAGAAGGGCGACACCTTCTCCTGCATGGTGGGCCACGAGGCCCTGCCGCTGGCCT | |
| TCACACAGAAGACCATCGACCGCTTGGCGGGTAAACCCACCCATGTCAATGTGTCTGTTG | |
| TCATGGTGGAGGTGGACGGCACCTGCTACTGAGCCGCCCGCCTGTCCCCACCCCTGAATA | |
| AACTCCATGCTCCCCCAAAAAAAAAAAAAAAAAAAAAAAAAAAAAAAAAAAAAAAAAAAA | |
| AAAAAAAAAAAAA | |

A NOV99 polypeptide (SEQ ID NO:322) encoded by SEQ ID NO:321 is 338 amino acids in length and is presented using the one-letter amino acid code in Table 99B. The Psort profile for NOV99 predicts that this sequence has no signal peptide and is likely to be localized to the cytoplasm with a certainty of 0.4500. In alternative embodiments, a NOV99 polypeptide is located to microbodies with a certainty of 0.1315.

TABLE 99B
NOV99 Polypeptide Sequence

| | |
|---|---|
| MAVYYCVSGGIYDSSGPFDYWGQGTLVTVSSASPTSPKVFPLSLCSTQPDGNVVIACLVQ | (SEQ ID NO:322) |
| GFFPQEPLSVTWSESCQGVTARNFPPSQDASGDLYTTSSQLTLPATQCLAGKSVTCHVKH | |
| YTNPSQDVTVPCPVPSTPPTPSPSTPPTPSPSCCHPRLSLHRPALEDLLLGSEANLTCTL | |
| TGLRDASGVTFTWTPSSGKSAVQGPPERDLCGCYSVSSVLPGCAQPWNHGETFTCTAAHP | |
| ELKTPLVRWLQGSQELPREKYLTWASRQEPSQGTTTFAVTSILRVAAEDWKKGDTFSCMV | |
| GHEALPLAFTQKTIDRLAGKPTHVNVSVVMVEVDGTCY | |

A BLAST analysis of NOV99 was run against the proprietary PatP GENESEQ Protein Patent database. It was found, for example, that the amino acid sequence of NOV99 had high homology to other proteins as shown in Table 99C.

TABLE 99C
BLASTX results from PatP database for NOV99

| | | Smallest |
|---|---|---|
| | | Sum |
| | High | Probability |
| Sequences producing High-scoring Segment Pairs: | Score | P(N) |
| patp: AAY88483 Cancer suppressor gene product | 1335 | 9.8e−187 |
| patp: AAB82914 Human immune response | 1268 | 1.2e−179 |
| protein HIRP3 | | |
| patp: AAM93283 Human polypeptide, | 1266 | 1.9e−179 |
| patp: AAY44723 Human immune system | 1262 | 5.1e−179 |
| molecule, ISMO-4 | | |
| patp: AAY96304 Human IGFAM-16 | 1254 | 3.6e−178 |
| immunoglobulin - Homo sapiens | | |

In a search of sequence databases, it was found, for example, that the nucleic acid sequence of this invention has 1122 of 1133 bases (99%) identical to a gb:GENBANK-ID:AF067420|acc:AF067420.1 mRNA from Homo sapiens (SNC73 protein (SNC73) mRNA). The full amino acid sequence of the protein of the invention was found to have 244 of 253 amino acid residues (96%) identical to, and 247 of 253 amino acid residues (97%) similar to, the 384 amino acid residue ptnr:SPTREMBL-ACC:Q9UP60 protein from Homo sapiens (Human) (SNC73 PROTEIN)(FIG. 3B). In addition to smaller changes, the sequence of this invention lacks 46 internal amino acids, when compared to ptnr:SPTREMBL-ACC:Q9UP60 protein from Homo sapiens (Human) (SNC73 PROTEIN). NOV99 also has homology to the other proteins shown in the BLASTP data in Table 99D.

TABLE 99D
NOV99 BLASTP results

| Gene Index/ | | Length | Identity | Positive | |
|---|---|---|---|---|---|
| Identifier | Protein/Organism | (aa) | (%) | (%) | Expect |
| gi\|229537\|prf\| | IgA H | 475 | 292/384 | 325/384 | e−168 |
| \|752400A | [Homo sapiens] | | (76) | (84) | |
| gi\|229585\|prf\| | IgA1 Bur | 686 | 298/383 | 323/383 | e−165 |
| \|763134A | [Homo sapiens] | | (77) | (83) | |
| gi\|223099\|prf\| | IgA-alpha1 Bur | 472 | 294/383 | 323/383 | e−165 |
| \|0506249A | [Homo sapiens] | | (76) | (83) | |
| gi\|3201900\|gb\| | SNC73 protein | 384 | 333/384 | 336/384 | e−155 |
| AAC19365.1\| | [Homo sapiens] | | (86) | (86) | |
| (AF067420) | | | | | |
| gi\|14042015\| | unnamed | 494 | 321/387 | 325/387 | e−150 |
| dbj\| | protein | | (82) | (83) | |
| BAB55072.1\| | product | | | | |
| (AK027379) | [Homo sapiens] | | | | |

This BLASTP data is displayed graphically in the ClustalW in Table 99E. A multiple sequence alignment is given, with the NOV99 protein being shown on line 1 in a ClustalW analysis comparing the protein of the invention with the related protein sequences shown in Table 99D.

[image]

[image]

[image]

Table 99F lists the domain description from DOMAIN analysis results against NOV99. This indicates that the NOV99 sequence has properties similar to those of other proteins known to contain this domain.

TABLE 99F
Domain Analysis of NOV99

gnl|Smart|smart00407, IGc1, Immunoglobulin C-Type SEQ ID NO:889
CD-Length = 75 residues, 96.0% aligned
Score = 46.6 bits (109), Expect = 2e−06

| | | | |
|---|---|---|---|
| NOV99: | 53 | VVIACLVQGFFPQEPLSVTWSESGQGVT--ARNFPPSQDASGDLYTTSSQLTLPATQCLA | 110 |
| | | + CLV GF+P ++VTW ++GQ VT + P +D G Y SS LT+ A+ + | |
| Sbjct: | 2 | ATLVCLVTGFYP-PDITVTWLKNGQEVTSGVKTTDPLKDKDG-TYFLSSYLTVSASTWES | 59 |
| NOV99: | 111 | GKSVTCHVKHYTNP | 124 |
| | | G TC V H | |
| Sbjct: | 60 | GDVYTCQVTHEGLT | 73 |

SNC73 was identified by subtractive hybridization between normal mucosa and colorectal cancer tissue as a gene which is down-regulated in colorectal cancer. It is highly homologous to the constant region of immunoglobulin alpha-1 chain. In higher vertebrates there are five classes of antibodies, IgA, IgD, IgE, IgG, and IgM, each with its own class of heavy chain—alpha, delta, epsilon, gamma, and mu, respectively. IgA molecules have alpha chains, IgG molecules have gamma chains, and so on. In addition, there are a number of subclasses of IgG and IgA immunoglobulins; for example, there are four human IgG subclasses (IgG1, IgG2, IgG3, and IgG4) having gamma1, gamma2, gamma3, and gamma4 heavy chains, respectively.

The various heavy chains impart a distinctive conformation to the hinge and tail regions of antibodies and give each class (and subclass) characteristic properties of its own. IgA is the principal class of antibody in secretions (saliva, tears, milk, and respiratory and intestinal secretions). It is transported through secretory epithelial cells from the extracellular fluid into the secreted fluid by the Poly Ig receptor, another type of Fc receptor that is unique to secretory epithelia. IgA serves both to defend against local infection and to prevent access of foreign antigens to the general immunologic system. This function is in accord with the potential role of SNC73 in colorectal cancer.

NOV99 is predicted to be expressed in at least the following tissues: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea and uterus. This information was derived by determining the tissue sources of the sequences that were included in the invention including but not limited to SeqCalling sources, public EST sources, literature sources, and/or RACE sources. Further expression data for NOV99 is provided in Example 2.

The NOV99 nucleic acids and proteins are useful in potential therapeutic applications implicated in various pathological disorders described further herein, for example, Von Hippel-Lindau (VHL) syndrome, Alzheimer's disease, stroke, tuberous sclerosis, hypercalceimia, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, neuroprotection, hemophilia, hypercoagulation, idiopathic thrombocytopenic purpura, immunodeficiencies, hemophilia, hypercoagulation, immunodeficiencies, graft vesus host disease as well as other diseases, disorders and conditions. NOV99 nucleic acids encoding the SNC73-like protein of the invention, or fragments thereof, may further be useful in diagnostic applications, wherein the presence or amount of the nucleic acid or the protein are to be assessed.

The novel nucleic acid of the invention encoding a SNC73-like protein includes the nucleic acid whose sequence is provided in Table 99A, or a fragment thereof. The invention also includes a mutant or variant nucleic acid any of whose bases may be changed from the corresponding base shown in Table 99A while still encoding a protein that maintains its SNC73-like activities and physiological functions, or a fragment of such a nucleic acid. The invention further includes nucleic acids whose sequences are complementary to the sequence of Table 99A, including nucleic acid fragments that are complementary to any of the nucleic acids just described.

The invention additionally includes nucleic acids or nucleic acid fragments, or complements thereto, whose structures include chemical modifications. Such modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 1% of the bases may be so changed.

The novel protein of the invention includes the SNC73-like protein whose sequence is provided in Table 99B. The invention also includes a mutant or variant protein any of whose residues may be changed from the corresponding residue shown in Table 99B while still encoding a protein that maintains its SNC73-like activities and physiological functions, or a functional fragment thereof. In the mutant or variant protein, up to about 4% of the amino acid residues may be so changed.

These materials are further useful in the generation of antibodies that bind immunospecifically to the substances of the invention for use in therapeutic or diagnostic methods. These antibodies may be generated according to methods known in the art, using prediction from hydrophobicity charts, as described in the “Anti-NOVX Antibodies” section below.

NOVX Nucleic Acids and Polypeptides

One aspect of the invention pertains to isolated nucleic acid molecules that encode NOVX polypeptides or biologically active portions thereof. Also included in the invention are nucleic acid fragments sufficient for use as hybridization probes to identify NOVX-encoding nucleic acids (e.g., NOVX mRNAs) and fragments for use as PCR primers for the amplification and/or mutation of NOVX nucleic acid molecules. As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule may be single-stranded or double-stranded, but preferably is comprised double-stranded DNA.

An NOVX nucleic acid can encode a mature NOVX polypeptide. As used herein, a “mature” form of a polypeptide or protein disclosed in the present invention is the product of a naturally occurring polypeptide or precursor form or proprotein. The naturally occurring polypeptide, precursor or proprotein includes, by way of nonlimiting example, the full-length gene product, encoded by the corresponding gene. Alternatively, it may be defined as the polypeptide, precursor or proprotein encoded by an ORF described herein. The product “mature” form arises, again by way of nonlimiting example, as a result of one or more naturally occurring processing steps as they may take place within the cell, or host cell, in which the gene product arises. Examples of such processing steps leading to a “mature” form of a polypeptide or protein include the cleavage of the N-terminal methionine residue encoded by the initiation codon of an ORF, or the proteolytic cleavage of a signal peptide or leader sequence. Thus a mature form arising from a precursor polypeptide or protein that has residues 1 to N, where residue 1 is the N-terminal methionine, would have residues 2 through N remaining after removal of the N-terminal methionine. Alternatively, a mature form arising from a precursor polypeptide or protein having residues 1 to N, in which an N-terminal signal sequence from residue 1 to residue M is cleaved, would have the residues from residue M+1 to residue N remaining. Further as used herein, a “mature” form of a polypeptide or protein may arise from a step of post-translational modification other than a proteolytic cleavage event. Such additional processes include, by way of non-limiting example, glycosylation, myristoylation or phosphorylation. In general, a mature polypeptide or protein may result from the operation of only one of these processes, or a combination of any of them.

The term “probes”, as utilized herein, refers to nucleic acid sequences of variable length, preferably between at least about 10 nucleotides (nt), 100 nt, or as many as approximately, e.g., 6,000 nt, depending upon the specific use. Probes are used in the detection of identical, similar, or complementary nucleic acid sequences. Longer length probes are generally obtained from a natural or recombinant source, are highly specific, and much slower to hybridize than shorter-length oligomer probes. Probes may be single- or double-stranded and designed to have specificity in PCR, membrane-based hybridization technologies, or ELISA-like technologies.

The term “isolated” nucleic acid molecule, as utilized herein, is one, which is separated from other nucleic acid molecules which are present in the natural source of the nucleic acid. Preferably, an “isolated” nucleic acid is free of sequences which naturally flank the nucleic acid (i.e., sequences located at the 5′- and 3′-termini of the nucleic acid) in the genomic DNA of the organism from which the nucleic acid is derived. For example, in various embodiments, the isolated NOVX nucleic acid molecules can contain less than about 5 kb, 4 kb, 3 kb, 2 kb, 1 kb, 0.5 kb or 0.1 kb of nucleotide sequences which naturally flank the nucleic acid molecule in genomic DNA of the cell/tissue from which the nucleic acid is derived (e.g., brain, heart, liver, spleen, etc.). Moreover, an “isolated” nucleic acid molecule, such as a cDNA molecule, can be substantially free of other cellular material or culture medium when produced by recombinant techniques, or of chemical precursors or other chemicals when chemically synthesized.

A nucleic acid molecule of the invention, e.g., a nucleic acid molecule having the nucleotide sequence SEQ ID NOS:2n−1, wherein n is an integer between 1 and 162, or a complement of this aforementioned nucleotide sequence, can be isolated using standard molecular biology techniques and the sequence information provided herein. Using all or a portion of the nucleic acid sequence of SEQ ID NOS:2n−1, wherein n is an integer between 1 and 162 as a hybridization probe, NOVX molecules can be isolated using standard hybridization and cloning techniques (e.g., as described in Sambrook, et al., (eds.), Molecular Cloning: A Laboratory Manual 2nd Ed., Cold Spring Harbor Laboratory Press, Cold Spring Harbor, N.Y., 1989; and Ausubel, et al., (eds.), Current Protocols in Molecular Biology, John Wiley & Sons, New York, N.Y., 1993.)

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to NOVX nucleotide sequences can be prepared by standard synthetic techniques, e.g., using an automated DNA synthesizer.

As used herein, the term “oligonucleotide” refers to a series of linked nucleotide residues, which oligonucleotide has a sufficient number of nucleotide bases to be used in a PCR reaction. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides comprise portions of a nucleic acid sequence having about 10 nt, 50 nt, or 100 nt in length, preferably about 15 nt to 30 nt in length. In one embodiment of the invention, an oligonucleotide comprising a nucleic acid molecule less than 100 nt in length would further comprise at least 6 contiguous nucleotides SEQ ID NOS:2n−1, wherein n is an integer between 1 and 162, or a complement thereof. Oligonucleotides may be chemically synthesized and may also be used as probes.

In another embodiment, an isolated nucleic acid molecule of the invention comprises a nucleic acid molecule that is a complement of the nucleotide sequence shown in SEQ ID NOS:2n−1, wherein n is an integer between 1 and 162, or a portion of this nucleotide sequence (e.g., a fragment that can be used as a probe or primer or a fragment encoding a biologically-active portion of an NOVX polypeptide). A nucleic acid molecule that is complementary to the nucleotide sequence shown SEQ ID NOS:2n−1, wherein n is an integer between 1 and 162 is one that is sufficiently complementary to the nucleotide sequence shown SEQ ID NOS:2n−1, wherein n is an integer between 1 and 162 that it can hydrogen bond with little or no mismatches to the nucleotide sequence shown SEQ ID NOS:2n−1, wherein n is an integer between 1 and 162, thereby forming a stable duplex.

As used herein, the term “complementary” refers to Watson-Crick or Hoogsteen base pairing between nucleotides units of a nucleic acid molecule, and the term “binding” means the physical or chemical interaction between two polypeptides or compounds or associated polypeptides or compounds or combinations thereof. Binding includes ionic, non-ionic, van der Waals, hydrophobic interactions, and the like. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

Fragments provided herein are defined as sequences of at least 6 (contiguous) nucleic acids or at least 4 (contiguous) amino acids, a length sufficient to allow for specific hybridization in the case of nucleic acids or for specific recognition of an epitope in the case of amino acids, respectively, and are at most some portion less than a full length sequence. Fragments may be derived from any contiguous portion of a nucleic acid or amino acid sequence of choice. Derivatives are nucleic acid sequences or amino acid sequences formed from the native compounds either directly or by modification or partial substitution. Analogs are nucleic acid sequences or amino acid sequences that have a structure similar to, but not identical to, the native compound but differs from it in respect to certain components or side chains. Analogs may be synthetic or from a different evolutionary origin and may have a similar or opposite metabolic activity compared to wild type. Homologs are nucleic acid sequences or amino acid sequences of a particular gene that are derived from different species.

Derivatives and analogs may be full length or other than full length, if the derivative or analog contains a modified nucleic acid or amino acid, as described below. Derivatives or analogs of the nucleic acids or proteins of the invention include, but are not limited to, molecules comprising regions that are substantially homologous to the nucleic acids or proteins of the invention, in various embodiments, by at least about 70%, 80%, or 95% identity (with a preferred identity of 80-95%) over a nucleic acid or amino acid sequence of identical size or when compared to an aligned sequence in which the alignment is done by a computer homology program known in the art, or whose encoding nucleic acid is capable of hybridizing to the complement of a sequence encoding the aforementioned proteins under stringent, moderately stringent, or low stringent conditions. See e.g. Ausubel, et al., Current Protocols in Molecular Biology, John Wiley & Sons, New York, N.Y., 1993, and below.

A “homologous nucleic acid sequence” or “homologous amino acid sequence,” or variations thereof, refer to sequences characterized by a homology at the nucleotide level or amino acid level as discussed above. Homologous nucleotide sequences encode those sequences coding for isoforms of NOVX polypeptides. Isoforms can be expressed in different tissues of the same organism as a result of, for example, alternative splicing of RNA. Alternatively, isoforms can be encoded by different genes. In the invention, homologous nucleotide sequences include nucleotide sequences encoding for an NOVX polypeptide of species other than humans, including, but not limited to: vertebrates, and thus can include, e.g., frog, mouse, rat, rabbit, dog, cat cow, horse, and other organisms. Homologous nucleotide sequences also include, but are not limited to, naturally occurring allelic variations and mutations of the nucleotide sequences set forth herein. A homologous nucleotide sequence does not, however, include the exact nucleotide sequence encoding human NOVX protein. Homologous nucleic acid sequences include those nucleic acid sequences that encode conservative amino acid substitutions (see below) in SEQ ID NOS:2n−1, wherein n is an integer between 1 and 162, as well as a polypeptide possessing NOVX biological activity. Various biological activities of the NOVX proteins are described below.

An NOVX polypeptide is encoded by the open reading frame (“ORF”) of an NOVX nucleic acid. An ORF corresponds to a nucleotide sequence that could potentially be translated into a polypeptide. A stretch of nucleic acids comprising an ORF is uninterrupted by a stop codon. An ORF that represents the coding sequence for a full protein begins with an ATG “start” codon and terminates with one of the three “stop” codons, namely, TAA, TAG, or TGA. For the purposes of this invention, an ORF may be any part of a coding sequence, with or without a start codon, a stop codon, or both. For an ORF to be considered as a good candidate for coding for a bona fide cellular protein, a minimum size requirement is often set, e.g., a stretch of DNA that would encode a protein of 50 amino acids or more.

The nucleotide sequences determined from the cloning of the human NOVX genes allows for the generation of probes and primers designed for use in identifying and/or cloning NOVX homologues in other cell types, e.g. from other tissues, as well as NOVX homologues from other vertebrates. The probe/primer typically comprises substantially purified oligonucleotide. The oligonucleotide typically comprises a region of nucleotide sequence that hybridizes under stringent conditions to at least about 12, 25, 50, 100, 150, 200, 250, 300, 350 or 400 consecutive sense strand nucleotide sequence SEQ ID NOS:2n−1, wherein n is an integer between 1 and 162; or an anti-sense strand nucleotide sequence of SEQ ID NOS:2n−1, wherein n is an integer between 1 and 162; or of a naturally occurring mutant of SEQ ID NOS:2n−1, wherein n is an integer between 1 and 162.

Probes based on the human NOVX nucleotide sequences can be used to detect transcripts or genomic sequences encoding the same or homologous proteins. In various embodiments, the probe further comprises a label group attached thereto, e.g. the label group can be a radioisotope, a fluorescent compound, an enzyme, or an enzyme co-factor. Such probes can be used as a part of a diagnostic test kit for identifying cells or tissues which mis-express an NOVX protein, such as by measuring a level of an NOVX-encoding nucleic acid in a sample of cells from a subject e.g., detecting NOVX mRNA levels or determining whether a genomic NOVX gene has been mutated or deleted.

“A polypeptide having a biologically-active portion of an NOVX polypeptide” refers to polypeptides exhibiting activity similar, but not necessarily identical to, an activity of a polypeptide of the invention, including mature forms, as measured in a particular biological assay, with or without dose dependency. A nucleic acid fragment encoding a “biologically-active portion of NOVX” can be prepared by isolating a portion SEQ ID NOS:2n−1, wherein n is an integer between 1 and 162, that encodes a polypeptide having an NOVX biological activity (the biological activities of the NOVX proteins are described below), expressing the encoded portion of NOVX protein (e.g., by recombinant expression in vitro) and assessing the activity of the encoded portion of NOVX.

NOVX Nucleic Acid and Polypeptide Variants

The invention further encompasses nucleic acid molecules that differ from the nucleotide sequences shown in SEQ ID NOS:2n−1, wherein n is an integer between 1 and 162 due to degeneracy of the genetic code and thus encode the same NOVX proteins as that encoded by the nucleotide sequences shown in SEQ ID NOS:2n−1, wherein n is an integer between 1 and 162. In another embodiment, an isolated nucleic acid molecule of the invention has a nucleotide sequence encoding a protein having an amino acid sequence shown in SEQ ID NOS:2n, wherein n is an integer between 1 and 162.

In addition to the human NOVX nucleotide sequences shown in SEQ ID NOS:2n−1, wherein n is an integer between 1 and 162, it will be appreciated by those skilled in the art that DNA sequence polymorphisms that lead to changes in the amino acid sequences of the NOVX polypeptides may exist within a population (e.g., the human population). Such genetic polymorphism in the NOVX genes may exist among individuals within a population due to natural allelic variation. As used herein, the terms “gene” and “recombinant gene” refer to nucleic acid molecules comprising an open reading frame (ORF) encoding an NOVX protein, preferably a vertebrate NOVX protein. Such natural allelic variations can typically result in 1-5% variance in the nucleotide sequence of the NOVX genes. Any and all such nucleotide variations and resulting amino acid polymorphisms in the NOVX polypeptides, which are the result of natural allelic variation and that do not alter the functional activity of the NOVX polypeptides, are intended to be within the scope of the invention.

Moreover, nucleic acid molecules encoding NOVX proteins from other species, and thus that have a nucleotide sequence that differs from the human SEQ ID NOS:2n−1, wherein n is an integer between 1 and 162 are intended to be within the scope of the invention. Nucleic acid molecules corresponding to natural allelic variants and homologues of the NOVX cDNAs of the invention can be isolated based on their homology to the human NOVX nucleic acids disclosed herein using the human cDNAs, or a portion thereof, as a hybridization probe according to standard hybridization techniques under stringent hybridization conditions.

Accordingly, in another embodiment, an isolated nucleic acid molecule of the invention is at least 6 nucleotides in length and hybridizes under stringent conditions to the nucleic acid molecule comprising the nucleotide sequence of SEQ ID NOS:2n−1, wherein n is an integer between 1 and 162. In another embodiment, the nucleic acid is at least 10, 25, 50, 100, 250, 500, 750, 1000, 1500, or 2000 or more nucleotides in length. In yet another embodiment, an isolated nucleic acid molecule of the invention hybridizes to the coding region. As used herein, the term “hybridizes under stringent conditions” is intended to describe conditions for hybridization and washing under which nucleotide sequences at least 60% homologous to each other typically remain hybridized to each other.

Homologs (i.e., nucleic acids encoding NOVX proteins derived from species other than human) or other related sequences (e.g., paralogs) can be obtained by low, moderate or high stringency hybridization with all or a portion of the particular human sequence as a probe using methods well known in the art for nucleic acid hybridization and cloning.

As used herein, the phrase “stringent hybridization conditions” refers to conditions under which a probe, primer or oligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Stringent conditions are known to those skilled in the art and can be found in Ausubel, et al., (eds.), Current Protocols in Molecular Biology, John Wiley & Sons, N.Y. (1989), 6.3.1-6.3.6. Preferably, the conditions are such that sequences at least about 65%, 70%, 75%, 85%, 90%, 95%, 98%, or 99% homologous to each other typically remain hybridized to each other. A non-limiting example of stringent hybridization conditions are hybridization in a high salt buffer comprising 6×SSC, 50 mM Tris-HCl (pH 7.5), 1 mM EDTA, 0.02% PVP, 0.02% Ficoll, 0.02% BSA, and 500 mg/ml denatured salmon sperm DNA at 65° C., followed by one or more washes in 0.2×SSC, 0.01% BSA at 50° C. An isolated nucleic acid molecule of the invention that hybridizes under stringent conditions to the sequences SEQ ID NOS:2n−1, wherein n is an integer between 1 and 162, corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In a second embodiment, a nucleic acid sequence that is hybridizable to the nucleic acid molecule comprising the nucleotide sequence of SEQ ID NOS:2n−1, wherein n is an integer between 1 and 162, or fragments, analogs or derivatives thereof, under conditions of moderate stringency is provided. A non-limiting example of moderate stringency hybridization conditions are hybridization in 6×SSC, 5× Denhardt's solution, 0.5% SDS and 100 mg/ml denatured salmon sperm DNA at 55° C., followed by one or more washes in 1×SSC, 0.1% SDS at 37° C. Other conditions of moderate stringency that may be used are well-known within the art. See, e.g., Ausubel, et al. (eds.), 1993, Current Protocols in Molecular Biology, John Wiley & Sons, NY, and Kriegler, 1990; Gene Transfer and Expression, A Laboratory Manual, Stockton Press, NY.

In a third embodiment, a nucleic acid that is hybridizable to the nucleic acid molecule comprising the nucleotide sequences SEQ ID NOS:2n−1, wherein n is an integer between 1 and 162, or fragments, analogs or derivatives thereof, under conditions of low stringency, is provided. A non-limiting example of low stringency hybridization conditions are hybridization in 35% formamide, 5×SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2×SSC, 25 mM Tris-HCl (pH 7.4), 5 mM EDTA, and 0.1% SDS at 50° C. Other conditions of low stringency that may be used are well known in the art (e.g., as employed for cross-species hybridizations). See, e.g., Ausubel, et al. (eds.), 1993, Current Protocols in Molecular Biology, John Wiley & Sons, NY, and Kriegler, 1990, Gene Transfer and Expression, A Laboratory Manual, Stockton Press, NY; Shilo and Weinberg, 1981. Proc Natl Acad Sci USA 78: 6789-6792.

Conservative Mutations

In addition to naturally-occurring allelic variants of NOVX sequences that may exist in the population, the skilled artisan will further appreciate that changes can be introduced by mutation into the nucleotide sequences SEQ ID NOS:2n−1, wherein n is an integer between 1 and 162, thereby leading to changes in the amino acid sequences of the encoded NOVX proteins, without altering the functional ability of said NOVX proteins. For example, nucleotide substitutions leading to amino acid substitutions at “non-essential” amino acid residues can be made in the sequence SEQ ID NOS:2n, wherein n is an integer between 1 and 162.A “non-essential” amino acid residue is a residue that can be altered from the wild-type sequences of the NOVX proteins without altering their biological activity, whereas an “essential” amino acid residue is required for such biological activity. For example, amino acid residues that are conserved among the NOVX proteins of the invention are predicted to be particularly non-amenable to alteration. Amino acids for which conservative substitutions can be made are well-known within the art.

Another aspect of the invention pertains to nucleic acid molecules encoding NOVX proteins that contain changes in amino acid residues that are not essential for activity. Such NOVX proteins differ in amino acid sequence from SEQ ID NOS:2n−1, wherein n is an integer between 1 and 162 yet retain biological activity. In one embodiment, the isolated nucleic acid molecule comprises a nucleotide sequence encoding a protein, wherein the protein comprises an amino acid sequence at least about 45% homologous to the amino acid sequences SEQ ID NOS:2n, wherein n is an integer between 1 and 162. Preferably, the protein encoded by the nucleic acid molecule is at least about 60% homologous to SEQ ID NOS:2n, wherein n is an integer between 1 and 162; more preferably at least about 70% homologous SEQ ID NOS:2n, wherein n is an integer between 1 and 162; still more preferably at least about 80% homologous to SEQ ID NOS:2n, wherein n is an integer between 1 and 162; even more preferably at least about 90% homologous to SEQ ID NOS:2n, wherein n is an integer between 1 and 162; and most preferably at least about 95% homologous to SEQ ID NOS:2n, wherein n is an integer between 1 and 162.

An isolated nucleic acid molecule encoding an NOVX protein homologous to the protein of SEQ ID NOS:2n, wherein n is an integer between 1 and 162 can be created by introducing one or more nucleotide substitutions, additions or deletions into the nucleotide sequence of SEQ ID NOS:2n−1, wherein n is an integer between 1 and 162, such that one or more amino acid substitutions, additions or deletions are introduced into the encoded protein.

Mutations can be introduced into SEQ ID NOS:2n−1, wherein n is an integer between 1 and 162 by standard techniques, such as site-directed mutagenesis and PCR-mediated mutagenesis. Preferably, conservative amino acid substitutions are made at one or more predicted, non-essential amino acid residues. A “conservative amino acid substitution” is one in which the amino acid residue is replaced with an amino acid residue having a similar side chain. Families of amino acid residues having similar side chains have been defined within the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted non-essential amino acid residue in the NOVX protein is replaced with another amino acid residue from the same side chain family. Alternatively, in another embodiment, mutations can be introduced randomly along all or part of an NOVX coding sequence, such as by saturation mutagenesis, and the resultant mutants can be screened for NOVX biological activity to identify mutants that retain activity. Following mutagenesis SEQ ID NOS:2n−1, wherein n is an integer between 1 and 162, the encoded protein can be expressed by any recombinant technology known in the art and the activity of the protein can be determined.

The relatedness of amino acid families may also be determined based on side chain interactions. Substituted amino acids may be fully conserved “strong” residues or fully conserved “weak” residues. The “strong” group of conserved amino acid residues may be any one of the following groups: STA, NEQK, NHQK, NDEQ, QHRK, MILV, MILF, HY, FYW, wherein the single letter amino acid codes are grouped by those amino acids that may be substituted for each other. Likewise, the “weak” group of conserved residues may be any one of the following: CSA, ATV, SAG, STNK, STPA, SGND, SNDEQK, NDEQHK, NEQHRK, VLIM, HFY, wherein the letters within each group represent the single letter amino acid code.

In one embodiment, a mutant NOVX protein can be assayed for (i) the ability to form protein:protein interactions with other NOVX proteins, other cell-surface proteins, or biologically-active portions thereof, (ii) complex formation between a mutant NOVX protein and an NOVX ligand; or (iii) the ability of a mutant NOVX protein to bind to an intracellular target protein or biologically-active portion thereof; (e.g. avidin proteins).

In yet another embodiment, a mutant NOVX protein can be assayed for the ability to regulate a specific biological function (e.g., regulation of insulin release).

Antisense Nucleic Acids

Another aspect of the invention pertains to isolated antisense nucleic acid molecules that are hybridizable to or complementary to the nucleic acid molecule comprising the nucleotide sequence of SEQ ID NOS:2n−1, wherein n is an integer between 1 and 162, or fragments, analogs or derivatives thereof. An “antisense” nucleic acid comprises a nucleotide sequence that is complementary to a “sense” nucleic acid encoding a protein (e.g., complementary to the coding strand of a double-stranded cDNA molecule or complementary to an mRNA sequence). In specific aspects, antisense nucleic acid molecules are provided that comprise a sequence complementary to at least about 10, 25, 50, 100, 250 or 500 nucleotides or an entire NOVX coding strand, or to only a portion thereof. Nucleic acid molecules encoding fragments, homologs, derivatives and analogs of an NOVX protein of SEQ ID NOS:2n, wherein n is an integer between 1 and 162, or antisense nucleic acids complementary to an NOVX nucleic acid sequence of SEQ ID NOS:2n−1, wherein n is an integer between 1 and 162, are additionally provided.

In one embodiment, an antisense nucleic acid molecule is antisense to a “coding region” of the coding strand of a nucleotide sequence encoding an NOVX protein. The term “coding region” refers to the region of the nucleotide sequence comprising codons which are translated into amino acid residues. In another embodiment, the antisense nucleic acid molecule is antisense to a “noncoding region” of the coding strand of a nucleotide sequence encoding the NOVX protein. The term “noncoding region” refers to 5′ and 3′ sequences which flank the coding region that are not translated into amino acids (i.e., also referred to as 5′ and 3′ untranslated regions).

Given the coding strand sequences encoding the NOVX protein disclosed herein, antisense nucleic acids of the invention can be designed according to the rules of Watson and Crick or Hoogsteen base pairing. The antisense nucleic acid molecule can be complementary to the entire coding region of NOVX mRNA, but more preferably is an oligonucleotide that is antisense to only a portion of the coding or noncoding region of NOVX mRNA. For example, the antisense oligonucleotide can be complementary to the region surrounding the translation start site of NOVX mRNA. An antisense oligonucleotide can be, for example, about 5, 10, 15, 20, 25, 30, 35, 40, 45 or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally-occurring nucleotides or variously modified nucleotides designed to increase the biological stability of the molecules or to increase the physical stability of the duplex formed between the antisense and sense nucleic acids (e.g., phosphorothioate derivatives and acridine substituted nucleotides can be used).

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding an NOVX protein to thereby inhibit expression of the protein (e.g., by inhibiting transcription and/or translation). The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface (e.g., by linking the antisense nucleic acid molecules to peptides or antibodies that bind to cell surface receptors or antigens). The antisense nucleic acid molecules can also be delivered to cells using the vectors described herein. To achieve sufficient nucleic acid molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other. See, e.g., Gaultier, et al., 1987. Nucl. Acids Res. 15: 6625-6641. The antisense nucleic acid molecule can also comprise a 2′-o-methylribonucleotide (See, e.g., Inoue, et al. 1987. Nucl. Acids Res. 15: 6131-6148) or a chimeric RNA-DNA analogue (See, e.g., Inoue, et al., 1987. FEBS Lett. 215: 327-330.

Ribozymes and PNA Moieties

Nucleic acid modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

In one embodiment, an antisense nucleic acid of the invention is a ribozyme. Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes as described in Haselhoff and Gerlach 1988. Nature 334: 585-591) can be used to catalytically cleave NOVX mRNA transcripts to thereby inhibit translation of NOVX mRNA. A ribozyme having specificity for an NOVX-encoding nucleic acid can be designed based upon the nucleotide sequence of an NOVX cDNA disclosed herein (i.e., SEQ ID NOS:2n−1, wherein n is an integer between 1 and 162). For example, a derivative of a Tetrahymena L-19 IVS RNA can be constructed in which the nucleotide sequence of the active site is complementary to the nucleotide sequence to be cleaved in an NOVX-encoding mRNA. See, e.g., U.S. Pat. No. 4,987,071 to Cech, et al. and U.S. Pat. No. 5,116,742 to Cech, et al. NOVX mRNA can also be used to select a catalytic RNA having a specific ribonuclease activity from a pool of RNA molecules. See, e.g., Bartel et al., (1993) Science 261:1411-1418.

Alternatively, NOVX gene expression can be inhibited by targeting nucleotide sequences complementary to the regulatory region of the NOVX nucleic acid (e.g., the NOVX promoter and/or enhancers) to form triple helical structures that prevent transcription of the NOVX gene in target cells. See, e.g., Helene, 1991. Anticancer Drug Des. 6: 569-84; Helene, et al. 1992. Ann. N.Y. Acad. Sci. 660: 27-36; Maher, 1992. Bioassays 14: 807-15.

In various embodiments, the NOVX nucleic acids can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids. See, e.g., Hyrup, et al., 1996. Bioorg Med Chem 4: 5-23.As used herein, the terms “peptide nucleic acids” or “PNAs” refer to nucleic acid mimics (e.g., DNA mimics) in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup, et al., 1996. supra; Perry-O'Keefe, et al., 1996. Proc. Natl. Acad. Sci. USA 93: 14670-14675.

PNAs of NOVX can be used in therapeutic and diagnostic applications. For example, PNAs can be used as antisense or antigene agents for sequence-specific modulation of gene expression by, e.g., inducing transcription or translation arrest or inhibiting replication. PNAs of NOVX can also be used, for example, in the analysis of single base pair mutations in a gene (e.g., PNA directed PCR clamping; as artificial restriction enzymes when used in combination with other enzymes, e.g., S₁ nucleases (See, Hyrup, et al., 1996.supra); or as probes or primers for DNA sequence and hybridization (See, Hyrup, et al., 1996, supra; Perry-O'Keefe, et al., 1996. supra).

In another embodiment, PNAs of NOVX can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of NOVX can be generated that may combine the advantageous properties of PNA and DNA. Such chimeras allow DNA recognition enzymes (e.g., Cleavage signal-1 protein H and DNA polymerases) to interact with the DNA portion while the PNA portion would provide high binding affinity and specificity. PNA-DNA chimeras can be linked using linkers of appropriate lengths selected in terms of base stacking, number of bonds between the nucleobases, and orientation (see, Hyrup, et al., 1996. supra). The synthesis of PNA-DNA chimeras can be performed as described in Hyrup, et al., 1996. supra and Finn, et al., 1996. Nucl Acids Res 24: 3357-3363. For example, a DNA chain can be synthesized on a solid support using standard phosphoramidite coupling chemistry, and modified nucleoside analogs, e.g., 5′-(4-methoxytrityl)amino-5′-deoxy-thymidine phosphoramidite, can be used between the PNA and the 5′ end of DNA. See, e.g., Mag, et al., 1989. Nucl Acid Res 17: 5973-5988. PNA monomers are then coupled in a stepwise manner to produce a chimeric molecule with a 5′ PNA segment and a 3′ DNA segment. See, e.g., Finn, et al., 1996. supra. Alternatively, chimeric molecules can be synthesized with a 5′ DNA segment and a 3′ PNA segment. See, e.g., Petersen, et al., 1975. Bioorg. Med. Chem. Lett. 5: 1119-11124.

In other embodiments, the oligonucleotide may include other appended groups such as peptides (e.g., for targeting host cell receptors in vivo), or agents facilitating transport across the cell membrane (see, e.g., Letsinger, et al., 1989. Proc. Natl. Acad. Sci. U.S.A. 86: 6553-6556; Lemaitre, et al., 1987. Proc. Natl. Acad. Sci. 84: 648-652; PCT Publication No. WO88/09810) or the blood-brain barrier (see, e.g., PCT Publication No. WO 89/10134). In addition, oligonucleotides can be modified with hybridization triggered cleavage agents (see, e.g., Krol, et al., 1988. BioTechniques 6:958-976) or intercalating agents (see, e.g., Zon, 1988. Pharm. Res. 5: 539-549). To this end, the oligonucleotide may be conjugated to another molecule, e.g., a peptide, a hybridization triggered cross-linking agent, a transport agent, a hybridization-triggered cleavage agent, and the like.

NOVX Polypeptides

A polypeptide according to the invention includes a polypeptide including the amino acid sequence of NOVX polypeptides whose sequences are provided in SEQ ID NOS:2n, wherein n is an integer between 1 and 162. The invention also includes a mutant or variant protein any of whose residues may be changed from the corresponding residues shown in SEQ ID NOS:2n, wherein n is an integer between 1 and 162 while still encoding a protein that maintains its NOVX activities and physiological functions, or a functional fragment thereof.

In general, an NOVX variant that preserves NOVX-like function includes any variant in which residues at a particular position in the sequence have been substituted by other amino acids, and further include the possibility of inserting an additional residue or residues between two residues of the parent protein as well as the possibility of deleting one or more residues from the parent sequence. Any amino acid substitution, insertion, or deletion is encompassed by the invention. In favorable circumstances, the substitution is a conservative substitution as defined above.

One aspect of the invention pertains to isolated NOVX proteins, and biologically-active portions thereof, or derivatives, fragments, analogs or homologs thereof. Also provided are polypeptide fragments suitable for use as immunogens to raise anti-NOVX antibodies. In one embodiment, native NOVX proteins can be isolated from cells or tissue sources by an appropriate purification scheme using standard protein purification techniques. In another embodiment, NOVX proteins are produced by recombinant DNA techniques. Alternative to recombinant expression, an NOVX protein or polypeptide can be synthesized chemically using standard peptide synthesis techniques.

An “isolated” or “purified” polypeptide or protein or biologically-active portion thereof is substantially free of cellular material or other contaminating proteins from the cell or tissue source from which the NOVX protein is derived, or substantially free from chemical precursors or other chemicals when chemically synthesized. The language “substantially free of cellular material” includes preparations of NOVX proteins in which the protein is separated from cellular components of the cells from which it is isolated or recombinantly-produced. In one embodiment, the language “substantially free of cellular material” includes preparations of NOVX proteins having less than about 30% (by dry weight) of non-NOVX proteins (also referred to herein as a “contaminating protein”), more preferably less than about 20% of non-NOVX proteins, still more preferably less than about 10% of non-NOVX proteins, and most preferably less than about 5% of non-NOVX proteins. When the NOVX protein or biologically-active portion thereof is recombinantly-produced, it is also preferably substantially free of culture medium, i.e., culture medium represents less than about 20%, more preferably less than about 10%, and most preferably less than about 5% of the volume of the NOVX protein preparation.

The language “substantially free of chemical precursors or other chemicals” includes preparations of NOVX proteins in which the protein is separated from chemical precursors or other chemicals that are involved in the synthesis of the protein. In one embodiment, the language “substantially free of chemical precursors or other chemicals” includes preparations of NOVX proteins having less than about 30% (by dry weight) of chemical precursors or non-NOVX chemicals, more preferably less than about 20% chemical precursors or non-NOVX chemicals, still more preferably less than about 10% chemical precursors or non-NOVX chemicals, and most preferably less than about 5% chemical precursors or non-NOVX chemicals.

Biologically-active portions of NOVX proteins include peptides comprising amino acid sequences sufficiently homologous to or derived from the amino acid sequences of the NOVX proteins (e.g., the amino acid sequence shown in SEQ ID NOS:2n, wherein n is an integer between 1 and 162) that include fewer amino acids than the full-length NOVX proteins, and exhibit at least one activity of an NOVX protein. Typically, biologically-active portions comprise a domain or motif with at least one activity of the NOVX protein. A biologically-active portion of an NOVX protein can be a polypeptide which is, for example, 10, 25, 50, 100 or more amino acid residues in length.

Moreover, other biologically-active portions, in which other regions of the protein are deleted, can be prepared by recombinant techniques and evaluated for one or more of the functional activities of a native NOVX protein.

In an embodiment, the NOVX protein has an amino acid sequence shown SEQ ID NOS:2n, wherein n is an integer between 1 and 162. In other embodiments, the NOVX protein is substantially homologous to SEQ ID NOS:2n, wherein n is an integer between 1 and 162, and retains the functional activity of the protein of SEQ ID NOS:2n, wherein n is an integer between 1 and 162, yet differs in amino acid sequence due to natural allelic variation or mutagenesis, as described in detail, below. Accordingly, in another embodiment, the NOVX protein is a protein that comprises an amino acid sequence at least about 45% homologous to the amino acid sequence SEQ ID NOS:2n, wherein n is an integer between 1 and 162, and retains the functional activity of the NOVX proteins of SEQ ID NOS:2n, wherein n is an integer between 1 and 162.

Determining Homology Between Two or More Sequences

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”).

The nucleic acid sequence homology may be determined as the degree of identity between two sequences. The homology may be determined using computer programs known in the art, such as GAP software provided in the GCG program package. See, Needleman and Wunsch, 1970. J Mol Biol 48: 443-453. Using GCG GAP software with the following settings for nucleic acid sequence comparison: GAP creation penalty of 5.0 and GAP extension penalty of 0.3, the coding region of the analogous nucleic acid sequences referred to above exhibits a degree of identity preferably of at least 70%, 75%, 80%, 85%, 90%, 95%, 98%, or 99%, with the CDS (encoding) part of the DNA sequence shown in SEQ ID NOS:2n−1, wherein n is an integer between 1 and 162.

The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g., A, T, C, G, U, or I, in the case of nucleic acids) occurs in both sequences to yield the number of matched positions, dividing the number of matched positions by the total number of positions in the region of comparison (i.e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region.

Chimeric and Fusion Proteins

The invention also provides NOVX chimeric or fusion proteins. As used herein, an NOVX “chimeric protein” or “fusion protein” comprises an NOVX polypeptide operatively-linked to a non-NOVX polypeptide. An “NOVX polypeptide” refers to a polypeptide having an amino acid sequence corresponding to an NOVX protein SEQ ID NOS:2n, wherein n is an integer between 1 and 162, whereas a “non-NOVX polypeptide” refers to a polypeptide having an amino acid sequence corresponding to a protein that is not substantially homologous to the NOVX protein, e.g., a protein that is different from the NOVX protein and that is derived from the same or a different organism. Within an NOVX fusion protein the NOVX polypeptide can correspond to all or a portion of an NOVX protein. In one embodiment, an NOVX fusion protein comprises at least one biologically-active portion of an NOVX protein. In another embodiment, an NOVX fusion protein comprises at least two biologically-active portions of an NOVX protein. In yet another embodiment, an NOVX fusion protein comprises at least three biologically-active portions of an NOVX protein. Within the fusion protein, the term “operatively-linked” is intended to indicate that the NOVX polypeptide and the non-NOVX polypeptide are fused in-frame with one another. The non-NOVX polypeptide can be fused to the N-terminus or C-terminus of the NOVX polypeptide.

In one embodiment, the fusion protein is a GST-NOVX fusion protein in which the NOVX sequences are fused to the C-terminus of the GST (glutathione S-transferase) sequences. Such fusion proteins can facilitate the purification of recombinant NOVX polypeptides.

In another embodiment, the fusion protein is an NOVX protein containing a heterologous signal sequence at its N-terminus. In certain host cells (e.g., mammalian host cells), expression and/or secretion of NOVX can be increased through use of a heterologous signal sequence.

In yet another embodiment, the fusion protein is an NOVX-immunoglobulin fusion protein in which the NOVX sequences are fused to sequences derived from a member of the immunoglobulin protein family. The NOVX-immunoglobulin fusion proteins of the invention can be incorporated into pharmaceutical compositions and administered to a subject to inhibit an interaction between an NOVX ligand and an NOVX protein on the surface of a cell, to thereby suppress NOVX-mediated signal transduction in vivo. The NOVX-immunoglobulin fusion proteins can be used to affect the bioavailability of an NOVX cognate ligand. Inhibition of the NOVX ligand/NOVX interaction may be useful therapeutically for both the treatment of proliferative and differentiative disorders, as well as modulating (e.g. promoting or inhibiting) cell survival. Moreover, the NOVX-immunoglobulin fusion proteins of the invention can be used as immunogens to produce anti-NOVX antibodies in a subject, to purify NOVX ligands, and in screening assays to identify molecules that inhibit the interaction of NOVX with an NOVX ligand.

An NOVX chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, e.g., Ausubel, et al. (eds.) Current Protocols in Molecular Biology, John Wiley & Sons, 1992). Moreover, many expression vectors are commercially available that already encode a fusion moiety (e.g., a GST polypeptide). An NOVX-encoding nucleic acid can be cloned into such an expression vector such that the fusion moiety is linked in-frame to the NOVX protein.

NOVX Agonists and Antagonists

The invention also pertains to variants of the NOVX proteins that function as either NOVX agonists (i.e., mimetics) or as NOVX antagonists. Variants of the NOVX protein can be generated by mutagenesis (e.g., discrete point mutation or truncation of the NOVX protein). An agonist of the NOVX protein can retain substantially the same, or a subset of, the biological activities of the naturally occurring form of the NOVX protein. An antagonist of the NOVX protein can inhibit one or more of the activities of the naturally occurring form of the NOVX protein by, for example, competitively binding to a downstream or upstream member of a cellular signaling cascade which includes the NOVX protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the NOVX proteins.

Variants of the NOVX proteins that function as either NOVX agonists (i.e., mimetics) or as NOVX antagonists can be identified by screening combinatorial libraries of mutants (e.g., truncation mutants) of the NOVX proteins for NOVX protein agonist or antagonist activity. In one embodiment, a variegated library of NOVX variants is generated by combinatorial mutagenesis at the nucleic acid level and is encoded by a variegated gene library. A variegated library of NOVX variants can be produced by, for example, enzymatically ligating a mixture of synthetic oligonucleotides into gene sequences such that a degenerate set of potential NOVX sequences is expressible as individual polypeptides, or alternatively, as a set of larger fusion proteins (e.g., for phage display) containing the set of NOVX sequences therein. There are a variety of methods which can be used to produce libraries of potential NOVX variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential NOVX sequences. Methods for synthesizing degenerate oligonucleotides are well-known within the art. See, e.g., Narang, 1983. Tetrahedron 39: 3; Itakura, et al., 1984. Annu. Rev. Biochem. 53: 323; Itakura, et al., 1984. Science 198: 1056; Ike, et al., 1983. Nucl. Acids Res. 11: 477.

Polypeptide Libraries

In addition, libraries of fragments of the NOVX protein coding sequences can be used to generate a variegated population of NOVX fragments for screening and subsequent selection of variants of an NOVX protein. In one embodiment, a library of coding sequence fragments can be generated by treating a double stranded PCR fragment of an NOVX coding sequence with a nuclease under conditions wherein nicking occurs only about once per molecule, denaturing the double stranded DNA, renaturing the DNA to form double-stranded DNA that can include sense/antisense pairs from different nicked products, removing single stranded portions from reformed duplexes by treatment with S₁ nuclease, and ligating the resulting fragment library into an expression vector. By this method, expression libraries can be derived which encodes N-terminal and internal fragments of various sizes of the NOVX proteins.

Various techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of NOVX proteins. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique that enhances the frequency of functional mutants in the libraries, can be used in combination with the screening assays to identify NOVX variants. See, e.g., Arkin and Yourvan, 1992. Proc. Natl. Acad. Sci. USA 89: 7811-7815; Delgrave, et al., 1993. Protein Engineering 6:327-331.

NOVX Antibodies

The term “antibody” as used herein refers to immunoglobulin molecules and immunologically active portions of immunoglobulin (Ig) molecules, i.e., molecules that contain an antigen binding site that specifically binds (immunoreacts with) an antigen. Such antibodies include, but are not limited to, polyclonal, monoclonal, chimeric, single chain, F_{ab}, F_{ab}, and F_{(ab′)2} fragments, and an F_{ab} expression library. In general, antibody molecules obtained from humans relates to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain. Reference herein to antibodies includes a reference to all such classes, subclasses and types of human antibody species.

An isolated protein of the invention intended to serve as an antigen, or a portion or fragment thereof, can be used as an immunogen to generate antibodies that immunospecifically bind the antigen, using standard techniques for polyclonal and monoclonal antibody preparation. The full-length protein can be used or, alternatively, the invention provides antigenic peptide fragments of the antigen for use as immunogens. An antigenic peptide fragment comprises at least 6 amino acid residues of the amino acid sequence of the full length protein, such as an amino acid sequence shown in SEQ ID NOs: 2n, wherein n is an integer between 1 and 162, and encompasses an epitope thereof such that an antibody raised against the peptide forms a specific immune complex with the full length protein or with any fragment that contains the epitope. Preferably, the antigenic peptide comprises at least 10 amino acid residues, or at least 15 amino acid residues, or at least 20 amino acid residues, or at least 30 amino acid residues. Preferred epitopes encompassed by the antigenic peptide are regions of the protein that are located on its surface; commonly these are hydrophilic regions.

In certain embodiments of the invention, at least one epitope encompassed by the antigenic peptide is a region of NOVX that is located on the surface of the protein, e.g., a hydrophilic region. A hydrophobicity analysis of the human NOVX protein sequence will indicate which regions of a NOVX polypeptide are particularly hydrophilic and, therefore, are likely to encode surface residues useful for targeting antibody production. As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation. See, e.g., Hopp and Woods, 1981, Proc. Nat. Acad. Sci. USA 78: 3824-3828; Kyte and Doolittle 1982, J. Mol. Biol. 157: 105-142, each incorporated herein by reference in their entirety. Antibodies that are specific for one or more domains within an antigenic protein, or derivatives, fragments, analogs or homologs thereof, are also provided herein.

A protein of the invention, or a derivative, fragment, analog, homolog or ortholog thereof, may be utilized as an immunogen in the generation of antibodies that immunospecifically bind these protein components.

Various procedures known within the art may be used for the production of polyclonal or monoclonal antibodies directed against a protein of the invention, or against derivatives, fragments, analogs homologs or orthologs thereof (see, for example, Antibodies: A Laboratory Manual, Harlow E, and Lane D, 1988, Cold Spring Harbor Laboratory Press, Cold Spring Harbor, N.Y., incorporated herein by reference). Some of these antibodies are discussed below.

Polyclonal Antibodies

For the production of polyclonal antibodies, various suitable host animals (e.g., rabbit, goat, mouse or other mammal) may be immunized by one or more injections with the native protein, a synthetic variant thereof, or a derivative of the foregoing. An appropriate immunogenic preparation can contain, for example, the naturally occurring immunogenic protein, a chemically synthesized polypeptide representing the immunogenic protein, or a recombinantly expressed immunogenic protein. Furthermore, the protein may be conjugated to a second protein known to be immunogenic in the mammal being immunized. Examples of such immunogenic proteins include but are not limited to keyhole limpet hemocyanin, serum albumin, bovine thyroglobulin, and soybean trypsin inhibitor. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), adjuvants usable in humans such as Bacille Calmette-Guerin and Corynebacterium parvum, or similar immunostimulatory agents. Additional examples of adjuvants which can be employed include MPL-TDM adjuvant (monophosphoryl Lipid A, synthetic trehalose dicorynomycolate).

The polyclonal antibody molecules directed against the immunogenic protein can be isolated from the mammal (e.g., from the blood) and further purified by well known techniques, such as affinity chromatography using protein A or protein G, which provide primarily the IgG fraction of immune serum. Subsequently, or alternatively, the specific antigen which is the target of the immunoglobulin sought, or an epitope thereof, may be immobilized on a column to purify the immune specific antibody by immunoaffinity chromatography. Purification of immunoglobulins is discussed, for example, by D. Wilkinson (The Scientist, published by The Scientist, Inc., Philadelphia Pa., Vol. 14, No. 8 (Apr. 17, 2000), pp. 25-28).

Monoclonal Antibodies

The term “monoclonal antibody” (MAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs thus contain an antigen binding site capable of immunoreacting with a particular epitope of the antigen characterized by a unique binding affinity for it.

Monoclonal antibodies can be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

The immunizing agent will typically include the protein antigen, a fragment thereof or a fusion protein thereof. Generally, either peripheral blood lymphocytes are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell [Goding, Monoclonal Antibodies: Principles and Practice, Academic Press, (1986) pp.59-103]. Immortalized cell lines are usually transformed mammalian cells, particularly myeloma cells of rodent, bovine and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies [Kozbor, J. Immunol., 133:3001 (1984); Brodeur et al., Monoclonal Antibody Production Techniques and Applications, Marcel Dekker, Inc., New York, (1987) pp. 51-63].

The culture medium in which the hybridoma cells are cultured can then be assayed for the presence of monoclonal antibodies directed against the antigen. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980). It is an objective, especially important in therapeutic applications of monoclonal antibodies, to identify antibodies having a high degree of specificity and a high binding affinity for the target antigen.

After the desired hybridoma cells are identified, the clones can be subcloned by limiting dilution procedures and grown by standard methods (Goding, 1986). Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies can also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (U.S. Pat. No. 4,816,567; Morrison, Nature 368, 812-13 (1994)) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

Humanized Antibodies

The antibodies directed against the protein antigens of the invention can further comprise humanized antibodies or human antibodies. These antibodies are suitable for administration to humans without engendering an immune response by the human against the administered immunoglobulin. Humanized forms of antibodies are chimeric immunoglobulins, immunoglobulin chains or fragments thereof (such as Fv, Fab, Fab′, F(ab′)₂ or other antigen-binding subsequences of antibodies) that are principally comprised of the sequence of a human immunoglobulin, and contain minimal sequence derived from a non-human immunoglobulin. Humanization can be performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. (See also U.S. Pat. No. 5,225,539.) In some instances, Fv framework residues of the human immunoglobulin are replaced by corresponding non-human residues. Humanized antibodies can also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the framework regions are those of a human immunoglobulin consensus sequence. The humanized antibody optimally also will comprise at least a portion of an immunoglobulin constant region (Fc), typically that of a human immunoglobulin (Jones et al., 1986; Riechmann et al., 1988; and Presta, Curr. Op. Struct. Biol., 2:593-596 (1992)).

Human Antibodies

Fully human antibodies essentially relate to antibody molecules in which the entire sequence of both the light chain and the heavy chain, including the CDRs, arise from human genes. Such antibodies are termed “human antibodies”, or “fully human antibodies” herein. Human monoclonal antibodies can be prepared by the trioma technique; the human B-cell hybridoma technique (see Kozbor, et al., 1983 Immunol Today 4: 72) and the EBV hybridoma technique to produce human monoclonal antibodies (see Cole, et al., 1985 In: Monoclonal Antibodies and Cancer Therapy, Alan R. Liss, Inc., pp. 77-96). Human monoclonal antibodies may be utilized in the practice of the present invention and may be produced by using human hybridomas (see Cote, et al., 1983. Proc Natl Acad Sci USA 80: 2026-2030) or by transforming human B-cells with Epstein Barr Virus in vitro (see Cole, et al., 1985 In: Monoclonal Antibodies and Cancer Therapy, Alan R. Liss, Inc., pp. 77-96).

In addition, human antibodies can also be produced using additional techniques, including phage display libraries (Hoogenboom and Winter, J. Mol. Biol., 227:381 (1991); Marks et al., J. Mol. Biol., 222:581 (1991)). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al. (Bio/Technology 10, 779-783 (1992)); Lonberg et al. (Nature 368 856-859 (1994)); Morrison (Nature 368, 812-13 (1994)); Fishwild et al,(Nature Biotechnology 14, 845-51 (1996)); Neuberger (Nature Biotechnology 14, 826 (1996)); and Lonberg and Huszar (Intern. Rev. Immunol. 13 65-93 (1995)).

Human antibodies may additionally be produced using transgenic nonhuman animals which are modified so as to produce fully human antibodies rather than the animal's endogenous antibodies in response to challenge by an antigen. (See PCT publication WO94/02602). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. The preferred embodiment of such a nonhuman animal is a mouse, and is termed the Xenomouse™ as disclosed in PCT publications WO 96/33735 and WO 96/34096. This animal produces B cells which secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv molecules.

An example of a method of producing a nonhuman host, exemplified as a mouse, lacking expression of an endogenous immunoglobulin heavy chain is disclosed in U.S. Pat. No. 5,939,598. It can be obtained by a method including deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker; and producing from the embryonic stem cell a transgenic mouse whose somatic and germ cells contain the gene encoding the selectable marker.

A method for producing an antibody of interest, such as a human antibody, is disclosed in U.S. Pat. No. 5,916,771. It includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

In a further improvement on this procedure, a method for identifying a clinically relevant epitope on an immunogen, and a correlative method for selecting an antibody that binds immunospecifically to the relevant epitope with high affinity, are disclosed in PCT publication WO 99/53049.

F_{ab} Fragments and Single Chain Antibodies

According to the invention, techniques can be adapted for the production of single-chain antibodies specific to an antigenic protein of the invention (see e.g., U.S. Pat. No. 4,946,778). In addition, methods can be adapted for the construction of F_{ab} expression libraries (see e.g., Huse, et al., 1989 Science 246: 1275-1281) to allow rapid and effective identification of monoclonal F_{ab} fragments with the desired specificity for a protein or derivatives, fragments, analogs or homologs thereof. Antibody fragments that contain the idiotypes to a protein antigen may be produced by techniques known in the art including, but not limited to: (i) an F_{(ab′)2} fragment produced by pepsin digestion of an antibody molecule; (ii) an F_{ab} fragment generated by reducing the disulfide bridges of an F_{(ab′)2} fragment; (iii) an F_{ab} fragment generated by the treatment of the antibody molecule with papain and a reducing agent and (iv) Fᵥ fragments.

Bispecific Antibodies

Bispecific antibodies are monoclonal, preferably human or humanized, antibodies that have binding specificities for at least two different antigens. In the present case, one of the binding specificities is for an antigenic protein of the invention. The second binding target is any other antigen, and advantageously is a cell-surface protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983)). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published May 13, 1993, and in Traunecker et al., EMBO J., 10:3655-3659 (1991).

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology, 121:210 (1986).

According to another approach described in WO 96/27011, the interface between a pair of antibody molecules can be engineered to maximize the percentage of heterodimers which are recovered from recombinant cell culture. The preferred interface comprises at least a part of the CH3 region of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies can be prepared as full length antibodies or antibody fragments (e.g. F(ab′)₂ bispecific antibodies). Techniques for generating bispecific antibodies from antibody fragments have been described in the literature. For example, bispecific antibodies can be prepared using chemical linkage. Brennan et al., Science 229:81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Additionally, Fab′ fragments can be directly recovered from E. coli and chemically coupled to form bispecific antibodies. Shalaby et al., J. Exp. Med. 175:217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al., J. Immunol. 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See, Gruber et al., J. Immunol. 152:5368 (1994).

Antibodies with more than two valencies are contemplated. For example, trispecific antibodies can be prepared. Tutt et al., J. Immunol. 147:60 (1991).

Exemplary bispecific antibodies can bind to two different epitopes, at least one of which originates in the protein antigen of the invention. Alternatively, an anti-antigenic arm of an immunoglobulin molecule can be combined with an arm which binds to a triggering molecule on a leukocyte such as a T-cell receptor molecule (e.g. CD2, CD3, CD28, or B7), or Fc receptors for IgG (FcγR), such as FcγRI (CD64), FcγRII (CD32) and FcγRIII (CD16) so as to focus cellular defense mechanisms to the cell expressing the particular antigen. Bispecific antibodies can also be used to direct cytotoxic agents to cells which express a particular antigen. These antibodies possess an antigen-binding arm and an arm which binds a cytotoxic agent or a radionuclide chelator, such as EOTUBE, DPTA, DOTA, or TETA. Another bispecific antibody of interest binds the protein antigen described herein and further binds tissue factor (TF).

Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells (U.S. Pat. No. 4,676,980), and for treatment of HIV infection (WO 91/00360; WO 92/200373; EP 03089). It is contemplated that the antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

Effector Function Engineering

It can be desirable to modify the antibody of the invention with respect to effector function, so as to enhance, e.g., the effectiveness of the antibody in treating cancer. For example, cysteine residue(s) can be introduced into the Fc region, thereby allowing interchain disulfide bond formation in this region. The homodimeric antibody thus generated can have improved internalization capability and/or increased complement-mediated cell killing and antibody-dependent cellular cytotoxicity (ADCC). See Caron et al., J. Exp Med., 176: 1191-1195 (1992) and Shopes, J. Immunol., 148: 2918-2922 (1992). Homodimeric antibodies with enhanced anti-tumor activity can also be prepared using heterobifunctional cross-linkers as described in Wolff et al. Cancer Research, 53: 2560-2565 (1993). Alternatively, an antibody can be engineered that has dual Fc regions and can thereby have enhanced complement lysis and ADCC capabilities. See Stevenson et al., Anti-Cancer Drug Design, 3: 219-230 (1989).

Immunoconjugates

The invention also pertains to immunoconjugates comprising an antibody conjugated to a cytotoxic agent such as a chemotherapeutic agent, toxin (e.g., an enzymatically active toxin of bacterial, fungal, plant, or animal origin, or fragments thereof), or a radioactive isotope (i.e., a radioconjugate).

Chemotherapeutic agents useful in the generation of such immunoconjugates have been described above. Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹I, ¹³¹In, ⁹⁰Y, and ¹⁸⁶Re.

Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis(p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science, 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026.

In another embodiment, the antibody can be conjugated to a “receptor” (such streptavidin) for utilization in tumor pretargeting wherein the antibody-receptor conjugate is administered to the patient, followed by removal of unbound conjugate from the circulation using a clearing agent and then administration of a “ligand” (e.g., avidin) that is in turn conjugated to a cytotoxic agent.

Immunoliposomes

The antibodies disclosed herein can also be formulated as immunoliposomes. Liposomes containing the antibody are prepared by methods known in the art, such as described in Epstein et al., Proc. Natl. Acad. Sci. USA, 82: 3688 (1985); Hwang et al., Proc. Natl Acad. Sci. USA, 77: 4030 (1980); and U.S. Pat. Nos. 4,485,045 and 4,544,545. Liposomes with enhanced circulation time are disclosed in U.S. Pat. No. 5,013,556.

Particularly useful liposomes can be generated by the reverse-phase evaporation method with a lipid composition comprising phosphatidylcholine, cholesterol, and PEG-derivatized phosphatidylethanolamine (PEG-PE). Liposomes are extruded through filters of defined pore size to yield liposomes with the desired diameter. Fab′ fragments of the antibody of the present invention can be conjugated to the liposomes as described in Martin et al., J. Biol. Chem., 257: 286-288 (1982) via a disulfide-interchange reaction. A chemotherapeutic agent (such as Doxorubicin) is optionally contained within the liposome. See Gabizon et al., J. National Cancer Inst., 81(19): 1484 (1989).

Diagnostic Applications of Antibodies Directed Against the Proteins of the Invention

Antibodies directed against a protein of the invention may be used in methods known within the art relating to the localization and/or quantitation of the protein (e.g., for use in measuring levels of the protein within appropriate physiological samples, for use in diagnostic methods, for use in imaging the protein, and the like). In a given embodiment, antibodies against the proteins, or derivatives, fragments, analogs or homologs thereof, that contain the antigen binding domain, are utilized as pharmacologically-active compounds (see below).

An antibody specific for a protein of the invention can be used to isolate the protein by standard techniques, such as immunoaffinity chromatography or immunoprecipitation. Such an antibody can facilitate the purification of the natural protein antigen from cells and of recombinantly produced antigen expressed in host cells. Moreover, such an antibody can be used to detect the antigenic protein (e.g., in a cellular lysate or cell supernatant) in order to evaluate the abundance and pattern of expression of the antigenic protein. Antibodies directed against the protein can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

Antibody Therapeutics

Antibodies of the invention, including polyclonal, monoclonal, humanized and fully human antibodies, may used as therapeutic agents. Such agents will generally be employed to treat or prevent a disease or pathology in a subject. An antibody preparation, preferably one having high specificity and high affinity for its target antigen, is administered to the subject and will generally have an effect due to its binding with the target. Such an effect may be one of two kinds, depending on the specific nature of the interaction between the given antibody molecule and the target antigen in question. In the first instance, administration of the antibody may abrogate or inhibit the binding of the target with an endogenous ligand to which it naturally binds. In this case, the antibody binds to the target and masks a binding site of the naturally occurring ligand, wherein the ligand serves as an effector molecule. Thus the receptor mediates a signal transduction pathway for which ligand is responsible.

Alternatively, the effect may be one in which the antibody elicits a physiological result by virtue of binding to an effector binding site on the target molecule. In this case the target, a receptor having an endogenous ligand which may be absent or defective in the disease or pathology, binds the antibody as a surrogate effector ligand, initiating a receptor-based signal transduction event by the receptor.

A therapeutically effective amount of an antibody of the invention relates generally to the amount needed to achieve a therapeutic objective. As noted above, this may be a binding interaction between the antibody and its target antigen that, in certain cases, interferes with the functioning of the target, and in other cases, promotes a physiological response. The amount required to be administered will furthermore depend on the binding affinity of the antibody for its specific antigen, and will also depend on the rate at which an administered antibody is depleted from the free volume other subject to which it is administered. Common ranges for therapeutically effective dosing of an antibody or antibody fragment of the invention may be, by way of nonlimiting example, from about 0.1 mg/kg body weight to about 50 mg/kg body weight. Common dosing frequencies may range, for example, from twice daily to once a week.

Pharmaceutical Compositions of Antibodies

Antibodies specifically binding a protein of the invention, as well as other molecules identified by the screening assays disclosed herein, can be administered for the treatment of various disorders in the form of pharmaceutical compositions. Principles and considerations involved in preparing such compositions, as well as guidance in the choice of components are provided, for example, in Remington: The Science And Practice Of Pharmacy 19th ed. (Alfonso R. Gennaro, et al., editors) Mack Pub. Co., Easton, Pa.: 1995; Drug Absorption Enhancement: Concepts, Possibilities, Limitations, And Trends, Harwood Academic Publishers, Langhorne, Pa., 1994; and Peptide And Protein Drug Delivery (Advances In Parenteral Sciences, Vol. 4), 1991, M. Dekker, New York.

If the antigenic protein is intracellular and whole antibodies are used as inhibitors, internalizing antibodies are preferred. However, liposomes can also be used to deliver the antibody, or an antibody fragment, into cells. Where antibody fragments are used, the smallest inhibitory fragment that specifically binds to the binding domain of the target protein is preferred. For example, based upon the variable-region sequences of an antibody, peptide molecules can be designed that retain the ability to bind the target protein sequence. Such peptides can be synthesized chemically and/or produced by recombinant DNA technology. See, e.g., Marasco et al., Proc. Natl. Acad. Sci. USA, 90: 7889-7893 (1993). The formulation herein can also contain more than one active compound as necessary for the particular indication being treated, preferably those with complementary activities that do not adversely affect each other. Alternatively, or in addition, the composition can comprise an agent that enhances its function, such as, for example, a cytotoxic agent, cytokine, chemotherapeutic agent, or growth-inhibitory agent. Such molecules are suitably present in combination in amounts that are effective for the purpose intended.

The active ingredients can also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles, and nanocapsules) or in macroemulsions.

The formulations to be used for in vivo administration must be sterile. This is readily accomplished by filtration through sterile filtration membranes.

Sustained-release preparations can be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods.

ELISA Assay

An agent for detecting an analyte protein is an antibody capable of binding to an analyte protein, preferably an antibody with a detectable label. Antibodies can be polyclonal, or more preferably, monoclonal. An intact antibody, or a fragment thereof (e.g., F_{ab} or F_{(ab)}₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. Included within the usage of the term “biological sample”, therefore, is blood and a fraction or component of blood including blood serum, blood plasma, or lymph. That is, the detection method of the invention can be used to detect an analyte mRNA, protein, or genomic DNA in a biological sample in vitro as well as in vivo. For example, in vitro techniques for detection of an analyte mRNA include Northern hybridizations and in situ hybridizations. In vitro techniques for detection of an analyte protein include enzyme linked immunosorbent assays (ELISAs), Western blots, immunoprecipitations, and immunofluorescence. In vitro techniques for detection of an analyte genomic DNA include Southern hybridizations. Procedures for conducting immunoassays are described, for example in “ELISA: Theory and Practice: Methods in Molecular Biology”, Vol. 42, J. R. Crowther (Ed.) Human Press, Totowa, N.J., 1995; “Immunoassay”, E. Diamandis and T. Christopoulus, Academic Press, Inc., San Diego, Calif., 1996; and “Practice and Thory of Enzyme Immunoassays”, P. Tijssen, Elsevier Science Publishers, Amsterdam, 1985. Furthermore, in vivo techniques for detection of an analyte protein include introducing into a subject a labeled anti-an analyte protein antibody. For example, the antibody can be labeled with a radioactive marker whose presence and location in a subject can be detected by standard imaging techniques.

NOVX Recombinant Expression Vectors and Host Cells

Another aspect of the invention pertains to vectors, preferably expression vectors, containing a nucleic acid encoding an NOVX protein, or derivatives, fragments, analogs or homologs thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell).

The term “regulatory sequence” is intended to includes promoters, enhancers and other expression control elements (e.g., polyadenylation signals). Such regulatory sequences are described, for example, in Goeddel, Gene Expression Technology: Methods in Enzymology 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., NOVX proteins, mutant forms of NOVX proteins, fusion proteins, etc.).

The recombinant expression vectors of the invention can be designed for expression of NOVX proteins in prokaryotic or eukaryotic cells. For example, NOVX proteins can be expressed in bacterial cells such as Escherichia coli, insect cells (using baculovirus expression vectors) yeast cells or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET 1d (Studier et al., Gene Expression Technology: Methods in Enzymology 185, Academic Press, San Diego, Calif. (1990) 60-89).

One strategy to maximize recombinant protein expression in E. coli is to express the protein in a host bacteria with an impaired capacity to proteolytically cleave the recombinant protein. See, e.g., Gottesman, Gene Expression Technology: Methods in Enzymology 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (see, e.g., Wada, et al., 1992. Nucl. Acids Res. 20: 2111-2118). Such alteration of nucleic acid sequences of the invention can be carried out by standard DNA synthesis techniques.

In another embodiment, the NOVX expression vector is a yeast expression vector. Examples of vectors for expression in yeast Saccharomyces cerivisae include pYepSec1 (Baldari, et al., 1987. EMBO J. 6: 229-234), pMFa (Kurjan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.).

Alternatively, NOVX can be expressed in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39).

In yet another embodiment, a nucleic acid of the invention is expressed in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are often provided by viral regulatory elements. For example, commonly used promoters are derived from polyoma, adenovirus 2, cytomegalovirus, and simian virus 40. For other suitable expression systems for both prokaryotic and eukaryotic cells see, e.g., Chapters 16 and 17 of Sambrook, et al., Molecular Cloning: A Laboratory Manual. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Banerji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the α-fetoprotein promoter (Campes and Tilghman, 1989. Genes Dev. 3: 537-546).

The invention further provides a recombinant expression vector comprising a DNA molecule of the invention cloned into the expression vector in an antisense orientation. That is, the DNA molecule is operatively-linked to a regulatory sequence in a manner that allows for expression (by transcription of the DNA molecule) of an RNA molecule that is antisense to NOVX mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see, e.g., Weintraub, et al., “Antisense RNA as a molecular tool for genetic analysis,” Reviews-Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, NOVX protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (Molecular Cloning: A Laboratory Manual. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding NOVX or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) NOVX protein. Accordingly, the invention further provides methods for producing NOVX protein using the host cells of the invention. In one embodiment, the method comprises culturing the host cell of invention (into which a recombinant expression vector encoding NOVX protein has been introduced) in a suitable medium such that NOVX protein is produced. In another embodiment, the method further comprises isolating NOVX protein from the medium or the host cell.

Transgenic NOVX Animals

The host cells of the invention can also be used to produce non-human transgenic animals. For example, in one embodiment, a host cell of the invention is a fertilized oocyte or an embryonic stem cell into which NOVX protein-coding sequences have been introduced. Such host cells can then be used to create non-human transgenic animals in which exogenous NOVX sequences have been introduced into their genome or homologous recombinant animals in which endogenous NOVX sequences have been altered. Such animals are useful for studying the function and/or activity of NOVX protein and for identifying and/or evaluating modulators of NOVX protein activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA that is integrated into the genome of a cell from which a transgenic animal develops and that remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous NOVX gene has been altered by homologous recombination between the endogenous gene and an exogenous DNA molecule introduced into a cell of the animal, e.g., an embryonic cell of the animal, prior to development of the animal.

A transgenic animal of the invention can be created by introducing NOVX-encoding nucleic acid into the male pronuclei of a fertilized oocyte (e.g., by microinjection, retroviral infection) and allowing the oocyte to develop in a pseudopregnant female foster animal. The human NOVX cDNA sequences SEQ ID NOS:2n−1, wherein n is an integer between 1 and 162 can be introduced as a transgene into the genome of a non-human animal. Alternatively, a non-human homologue of the human NOVX gene, such as a mouse NOVX gene, can be isolated based on hybridization to the human NOVX cDNA (described further supra) and used as a transgene. Intronic sequences and polyadenylation signals can also be included in the transgene to increase the efficiency of expression of the transgene. A tissue-specific regulatory sequence(s) can be operably-linked to the NOVX transgene to direct expression of NOVX protein to particular cells. Methods for generating transgenic animals via embryo manipulation and microinjection, particularly animals such as mice, have become conventional in the art and are described, for example, in U.S. Pat. Nos. 4,736,866; 4,870,009; and 4,873,191; and Hogan, 1986. In: Manipulating the Mouse Embryo, Cold Spring Harbor Laboratory Press, Cold Spring Harbor, N.Y. Similar methods are used for production of other transgenic animals. A transgenic founder animal can be identified based upon the presence of the NOVX transgene in its genome and/or expression of NOVX mRNA in tissues or cells of the animals. A transgenic founder animal can then be used to breed additional animals carrying the transgene. Moreover, transgenic animals carrying a transgene-encoding NOVX protein can further be bred to other transgenic animals carrying other transgenes.

To create a homologous recombinant animal, a vector is prepared which contains at least a portion of an NOVX gene into which a deletion, addition or substitution has been introduced to thereby alter, e.g., functionally disrupt, the NOVX gene. The NOVX gene can be a human gene (e.g., the cDNA of SEQ ID NOS:2n−1, wherein n is an integer between 1 and 162), but more preferably, is a non-human homologue of a human NOVX gene. For example, a mouse homologue of human NOVX gene of SEQ ID NOS:2n−1, wherein n is an integer between 1 and 162 can be used to construct a homologous recombination vector suitable for altering an endogenous NOVX gene in the mouse genome. In one embodiment, the vector is designed such that, upon homologous recombination, the endogenous NOVX gene is functionally disrupted (i.e., no longer encodes a functional protein; also referred to as a “knock out” vector).

Alternatively, the vector can be designed such that, upon homologous recombination, the endogenous NOVX gene is mutated or otherwise altered but still encodes functional protein (e.g., the upstream regulatory region can be altered to thereby alter the expression of the endogenous NOVX protein). In the homologous recombination vector, the altered portion of the NOVX gene is flanked at its 5′- and 3′-termini by additional nucleic acid of the NOVX gene to allow for homologous recombination to occur between the exogenous NOVX gene carried by the vector and an endogenous NOVX gene in an embryonic stem cell. The additional flanking NOVX nucleic acid is of sufficient length for successful homologous recombination with the endogenous gene. Typically, several kilobases of flanking DNA (both at the 5′- and 3′-termini) are included in the vector. See, e.g., Thomas, et al., 1987. Cell 51: 503 for a description of homologous recombination vectors. The vector is ten introduced into an embryonic stem cell line (e.g., by electroporation) and cells in which the introduced NOVX gene has homologously-recombined with the endogenous NOVX gene are selected. See, e.g., Li, et al., 1992. Cell 69: 915.

The selected cells are then injected into a blastocyst of an animal (e.g., a mouse) to form aggregation chimeras. See, e.g., Bradley, 1987. In: Teratocarcinomas and Embryonic Stem Cells: A Practical Approach, Robertson, ed. IRL, Oxford, pp. 113-152. A chimeric embryo can then be implanted into a suitable pseudopregnant female foster animal and the embryo brought to term. Progeny harboring the homologously-recombined DNA in their germ cells can be used to breed animals in which all cells of the animal contain the homologously-recombined DNA by germline transmission of the transgene. Methods for constructing homologous recombination vectors and homologous recombinant animals are described further in Bradley, 1991. Curr. Opin. Biotechnol. 2: 823-829; PCT International Publication Nos.: WO 90/11354; WO 91/01140; WO 92/0968; and WO 93/04169.

In another embodiment, transgenic non-humans animals can be produced that contain selected systems that allow for regulated expression of the transgene. One example of such a system is the cre/loxP recombinase system of bacteriophage P1. For a description of the cre/loxP recombinase system, See, e.g., Lakso, et al., 1992. Proc. Natl. Acad. Sci. USA 89: 6232-6236.Another example of a recombinase system is the FLP recombinase system of Saccharomyces cerevisiae. See, O'Gorman, et al., 1991. Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, et al., 1997. Nature 385: 810-813. In brief, a cell (e.g., a somatic cell) from the transgenic animal can be isolated and induced to exit the growth cycle and enter G₀ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell (e.g., the somatic cell) is isolated.

Pharmaceutical Compositions

The NOVX nucleic acid molecules, NOVX proteins, and anti-NOVX antibodies (also referred to herein as “active compounds”) of the invention, and derivatives, fragments, analogs and homologs thereof, can be incorporated into pharmaceutical compositions suitable for administration. Such compositions typically comprise the nucleic acid molecule, protein, or antibody and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., an NOVX protein or anti-NOVX antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No.4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see, e.g., U.S. Pat. No. 5,328,470) or by stereotactic injection (see, e.g., Chen, et al., 1994. Proc. Natl. Acad. Sci. USA 91: 3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

The pharmaceutical compositions can be included in a container, pack, or dispenser together with instructions for administration.

Screening and Detection Methods

The isolated nucleic acid molecules of the invention can be used to express NOVX protein (e.g., via a recombinant expression vector in a host cell in gene therapy applications), to detect NOVX mRNA (e.g., in a biological sample) or a genetic lesion in an NOVX gene, and to modulate NOVX activity, as described further, below. In addition, the NOVX proteins can be used to screen drugs or compounds that modulate the NOVX protein activity or expression as well as to treat disorders characterized by insufficient or excessive production of NOVX protein or production of NOVX protein forms that have decreased or aberrant activity compared to NOVX wild-type protein (e.g.; diabetes (regulates insulin release); obesity (binds and transport lipids); metabolic disturbances associated with obesity, the metabolic syndrome X as well as anorexia and wasting disorders associated with chronic diseases and various cancers, and infectious disease(possesses anti-microbial activity) and the various dyslipidemias. In addition, the anti-NOVX antibodies of the invention can be used to detect and isolate NOVX proteins and modulate NOVX activity. In yet a further aspect, the invention can be used in methods to influence appetite, absorption of nutrients and the disposition of metabolic substrates in both a positive and negative fashion.

The invention further pertains to novel agents identified by the screening assays described herein and uses thereof for treatments as described, supra.

Screening Assays

The invention provides a method (also referred to herein as a “screening assay”) for identifying modulators, i.e., candidate or test compounds or agents (e.g., peptides, peptidomimetics, small molecules or other drugs) that bind to NOVX proteins or have a stimulatory or inhibitory effect on, e.g., NOVX protein expression or NOVX protein activity. The invention also includes compounds identified in the screening assays described herein.

In one embodiment, the invention provides assays for screening candidate or test compounds which bind to or modulate the activity of the membrane-bound form of an NOVX protein or polypeptide or biologically-active portion thereof. The test compounds of the invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds. See, e.g., Lam, 1997. Anticancer Drug Design 12: 145.

A “small molecule” as used herein, is meant to refer to a composition that has a molecular weight of less than about 5 kD and most preferably less than about 4 kD. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention.

Examples of methods for the synthesis of molecular libraries can be found in the art, for example in: DeWitt, et al., 1993. Proc. Natl. Acad. Sci. U.S.A. 90: 6909; Erb, et al., 1994. Proc. Natl. Acad. Sci. U.S.A. 91: 11422; Zuckermann, et al., 1994. J. Med. Chem. 37: 2678; Cho, et al., 1993. Science 261: 1303; Carrell, et al., 1994. Angew. Chem. Int. Ed. Engl. 33: 2059; Carell, et al., 1994. Angew. Chem. Int. Ed. Engl. 33: 2061; and Gallop, et al., 1994. J. Med. Chem. 37: 1233.

Libraries of compounds may be presented in solution (e.g., Houghten, 1992. Biotechniques 13: 412-421), or on beads (Lam, 1991. Nature 354: 82-84), on chips (Fodor, 1993. Nature 364: 555-556), bacteria (Ladner, U.S. Pat. No. 5,223,409), spores (Ladner, U.S. Pat. No. 5,233,409), plasmids (Cull, et al., 1992. Proc. Natl. Acad. Sci. USA 89: 1865-1869) or on phage (Scott and Smith, 1990. Science 249: 386-390; Devlin, 1990. Science 249: 404-406; Cwirla, et al., 1990. Proc. Natl. Acad. Sci. U.S.A. 87: 6378-6382; Felici, 1991. J. Mol. Biol. 222: 301-310; Ladner, U.S. Pat. No. 5,233,409.).

In one embodiment, an assay is a cell-based assay in which a cell which expresses a membrane-bound form of NOVX protein, or a biologically-active portion thereof, on the cell surface is contacted with a test compound and the ability of the test compound to bind to an NOVX protein determined. The cell, for example, can of mammalian origin or a yeast cell. Determining the ability of the test compound to bind to the NOVX protein can be accomplished, for example, by coupling the test compound with a radioisotope or enzymatic label such that binding of the test compound to the NOVX protein or biologically-active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioemission or by scintillation counting. Alternatively, test compounds can be enzymatically-labeled with, for example, horseradish peroxidase, alkaline phosphatase, or luciferase, and the enzymatic label detected by determination of conversion of an appropriate substrate to product. In one embodiment, the assay comprises contacting a cell which expresses a membrane-bound form of NOVX protein, or a biologically-active portion thereof, on the cell surface with a known compound which binds NOVX to form an assay mixture, contacting the assay mixture with a test compound, and determining the ability of the test compound to interact with an NOVX protein, wherein determining the ability of the test compound to interact with an NOVX protein comprises determining the ability of the test compound to preferentially bind to NOVX protein or a biologically-active portion thereof as compared to the known compound.

In another embodiment, an assay is a cell-based assay comprising contacting a cell expressing a membrane-bound form of NOVX protein, or a biologically-active portion thereof, on the cell surface with a test compound and determining the ability of the test compound to modulate (e.g., stimulate or inhibit) the activity of the NOVX protein or biologically-active portion thereof. Determining the ability of the test compound to modulate the activity of NOVX or a biologically-active portion thereof can be accomplished, for example, by determining the ability of the NOVX protein to bind to or interact with an NOVX target molecule. As used herein, a “target molecule” is a molecule with which an NOVX protein binds or interacts in nature, for example, a molecule on the surface of a cell which expresses an NOVX interacting protein, a molecule on the surface of a second cell, a molecule in the extracellular milieu, a molecule associated with the internal surface of a cell membrane or a cytoplasmic molecule. An NOVX target molecule can be a non-NOVX molecule or an NOVX protein or polypeptide of the invention. In one embodiment, an NOVX target molecule is a component of a signal transduction pathway that facilitates transduction of an extracellular signal (e.g. a signal generated by binding of a compound to a membrane-bound NOVX molecule) through the cell membrane and into the cell. The target, for example, can be a second intercellular protein that has catalytic activity or a protein that facilitates the association of downstream signaling molecules with NOVX.

Determining the ability of the NOVX protein to bind to or interact with an NOVX target molecule can be accomplished by one of the methods described above for determining direct binding. In one embodiment, determining the ability of the NOVX protein to bind to or interact with an NOVX target molecule can be accomplished by determining the activity of the target molecule. For example, the activity of the target molecule can be determined by detecting induction of a cellular second messenger of the target (i.e. intracellular Ca²⁺, diacylglycerol, IP₃, etc.), detecting catalytic/enzymatic activity of the target an appropriate substrate, detecting the induction of a reporter gene (comprising an NOVX-responsive regulatory element operatively linked to a nucleic acid encoding a detectable marker, e.g., luciferase), or detecting a cellular response, for example, cell survival, cellular differentiation, or cell proliferation.

In yet another embodiment, an assay of the invention is a cell-free assay comprising contacting an NOVX protein or biologically-active portion thereof with a test compound and determining the ability of the test compound to bind to the NOVX protein or biologically-active portion thereof. Binding of the test compound to the NOVX protein can be determined either directly or indirectly as described above. In one such embodiment, the assay comprises contacting the NOVX protein or biologically-active portion thereof with a known compound which binds NOVX to form an assay mixture, contacting the assay mixture with a test compound, and determining the ability of the test compound to interact with an NOVX protein, wherein determining the ability of the test compound to interact with an NOVX protein comprises determining the ability of the test compound to preferentially bind to NOVX or biologically-active portion thereof as compared to the known compound.

In still another embodiment, an assay is a cell-free assay comprising contacting NOVX protein or biologically-active portion thereof with a test compound and determining the ability of the test compound to modulate (e.g. stimulate or inhibit) the activity of the NOVX protein or biologically-active portion thereof. Determining the ability of the test compound to modulate the activity of NOVX can be accomplished, for example, by determining the ability of the NOVX protein to bind to an NOVX target molecule by one of the methods described above for determining direct binding. In an alternative embodiment, determining the ability of the test compound to modulate the activity of NOVX protein can be accomplished by determining the ability of the NOVX protein further modulate an NOVX target molecule. For example, the catalytic/enzymatic activity of the target molecule on an appropriate substrate can be determined as described, supra.

In yet another embodiment, the cell-free assay comprises contacting the NOVX protein or biologically-active portion thereof with a known compound which binds NOVX protein to form an assay mixture, contacting the assay mixture with a test compound, and determining the ability of the test compound to interact with an NOVX protein, wherein determining the ability of the test compound to interact with an NOVX protein comprises determining the ability of the NOVX protein to preferentially bind to or modulate the activity of an NOVX target molecule.

The cell-free assays of the invention are amenable to use of both the soluble form or the membrane-bound form of NOVX protein. In the case of cell-free assays comprising the membrane-bound form of NOVX protein, it may be desirable to utilize a solubilizing agent such that the membrane-bound form of NOVX protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, N-dodecyl-N,N-dimethyl-3-ammonio-1-propane sulfonate, 3-(3-cholamidopropyl) dimethylamminiol-1-propane sulfonate (CHAPS), or 3-(3-cholamidopropyl)dimethylamminiol-2-hydroxy-1-propane sulfonate (CHAPSO).

In more than one embodiment of the above assay methods of the invention, it may be desirable to immobilize either NOVX protein or its target molecule to facilitate separation of complexed from uncomplexed forms of one or both of the proteins, as well as to accommodate automation of the assay. Binding of a test compound to NOVX protein, or interaction of NOVX protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, GST-NOVX fusion proteins or GST-target fusion proteins can be adsorbed onto glutathione sepharose beads (Sigma Chemical, St. Louis, Mo.) or glutathione derivatized microtiter plates, that are then combined with the test compound or the test compound and either the non-adsorbed target protein or NOVX protein, and the mixture is incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described, supra. Alternatively, the complexes can be dissociated from the matrix, and the level of NOVX protein binding or activity determined using standard techniques.

Other techniques for immobilizing proteins on matrices can also be used in the screening assays of the invention. For example, either the NOVX protein or its target molecule can be immobilized utilizing conjugation of biotin and streptavidin. Biotinylated NOVX protein or target molecules can be prepared from biotin-NHS (N-hydroxy-succinimide) using techniques well-known within the art (e.g., biotinylation kit, Pierce Chemicals, Rockford, Ill.), and immobilized in the wells of streptavidin-coated 96 well plates (Pierce Chemical). Alternatively, antibodies reactive with NOVX protein or target molecules, but which do not interfere with binding of the NOVX protein to its target molecule, can be derivatized to the wells of the plate, and unbound target or NOVX protein trapped in the wells by antibody conjugation. Methods for detecting such complexes, in addition to those described above for the GST-immobilized complexes, include immunodetection of complexes using antibodies reactive with the NOVX protein or target molecule, as well as enzyme-linked assays that rely on detecting an enzymatic activity associated with the NOVX protein or target molecule.

In another embodiment, modulators of NOVX protein expression are identified in a method wherein a cell is contacted with a candidate compound and the expression of NOVX mRNA or protein in the cell is determined. The level of expression of NOVX mRNA or protein in the presence of the candidate compound is compared to the level of expression of NOVX mRNA or protein in the absence of the candidate compound. The candidate compound can then be identified as a modulator of NOVX mRNA or protein expression based upon this comparison. For example, when expression of NOVX mRNA or protein is greater (i.e., statistically significantly greater) in the presence of the candidate compound than in its absence, the candidate compound is identified as a stimulator of NOVX mRNA or protein expression. Alternatively, when expression of NOVX mRNA or protein is less (statistically significantly less) in the presence of the candidate compound than in its absence, the candidate compound is identified as an inhibitor of NOVX mRNA or protein expression. The level of NOVX mRNA or protein expression in the cells can be determined by methods described herein for detecting NOVX mRNA or protein.

In yet another aspect of the invention, the NOVX proteins can be used as “bait proteins” in a two-hybrid assay or three hybrid assay (see, e.g., U.S. Pat. No. 5,283,317; Zervos, et al., 1993. Cell 72: 223-232; Madura, et al., 1993. J. Biol. Chem. 268: 12046-12054; Bartel, et al., 1993. Biotechniques 14: 920-924; Iwabuchi, et al., 1993. Oncogene 8: 1693-1696; and Brent WO 94/10300), to identify other proteins that bind to or interact with NOVX (“NOVX-binding proteins” or “NOVX-bp”) and modulate NOVX activity. Such NOVX-binding proteins are also likely to be involved in the propagation of signals by the NOVX proteins as, for example, upstream or downstream elements of the NOVX pathway.

The two-hybrid system is based on the modular nature of most transcription factors, which consist of separable DNA-binding and activation domains. Briefly, the assay utilizes two different DNA constructs. In one construct, the gene that codes for NOVX is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming an NOVX-dependent complex, the DNA-binding and activation domains of the transcription factor are brought into close proximity. This proximity allows transcription of a reporter gene (e.g., LacZ) that is operably linked to a transcriptional regulatory site responsive to the transcription factor. Expression of the reporter gene can be detected and cell colonies containing the functional transcription factor can be isolated and used to obtain the cloned gene that encodes the protein which interacts with NOVX.

The invention further pertains to novel agents identified by the aforementioned screening assays and uses thereof for treatments as described herein.

Detection Assays

Portions or fragments of the cDNA sequences identified herein (and the corresponding complete gene sequences) can be used in numerous ways as polynucleotide reagents. By way of example, and not of limitation, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. Some of these applications are described in the subsections, below.

Chromosome Mapping

Once the sequence (or a portion of the sequence) of a gene has been isolated, this sequence can be used to map the location of the gene on a chromosome. This process is called chromosome mapping. Accordingly, portions or fragments of the NOVX sequences, SEQ ID NOS:2n−1, wherein n is an integer between 1 and 162, or fragments or derivatives thereof, can be used to map the location of the NOVX genes, respectively, on a chromosome. The mapping of the NOVX sequences to chromosomes is an important first step in correlating these sequences with genes associated with disease.

Briefly, NOVX genes can be mapped to chromosomes by preparing PCR primers (preferably 15-25 bp in length) from the NOVX sequences. Computer analysis of the NOVX, sequences can be used to rapidly select primers that do not span more than one exon in the genomic DNA, thus complicating the amplification process. These primers can then be used for PCR screening of somatic cell hybrids containing individual human chromosomes. Only those hybrids containing the human gene corresponding to the NOVX sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but in which human cells can, the one human chromosome that contains the gene encoding the needed enzyme will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. See, e.g., D'Eustachio, et al., 1983. Science 220: 919-924. Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the NOVX sequences to design oligonucleotide primers, sub-localization can be achieved with panels of fragments from specific chromosomes.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical like colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases, will suffice to get good results at a reasonable amount of time. For a review of this technique, see, Verma, et al., Human Chromosomes: A Manual of Basic Techniques (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. Such data are found, e.g., in McKusick, Mendelian Inheritance in Man, available on-line through Johns Hopkins University Welch Medical Library). The relationship between genes and disease, mapped to the same chromosomal region, can then be identified through linkage analysis (co-inheritance of physically adjacent genes), described in, e.g., Egeland, et al., 1987. Nature, 325: 783-787.

Moreover, differences in the DNA sequences between individuals affected and unaffected with a disease associated with the NOVX gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

Tissue Typing

The NOVX sequences of the invention can also be used to identify individuals from minute biological samples. In this technique, an individual's genomic DNA is digested with one or more restriction enzymes, and probed on a Southern blot to yield unique bands for identification. The sequences of the invention are useful as additional DNA markers for RFLP (“restriction fragment length polymorphisms,” described in U.S. Pat. No. 5,272,057).

Furthermore, the sequences of the invention can be used to provide an alternative technique that determines the actual base-by-base DNA sequence of selected portions of an individual's genome. Thus, the NOVX sequences described herein can be used to prepare two PCR primers from the 5′- and 3′-termini of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the invention can be used to obtain such identification sequences from individuals and from tissue. The NOVX sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Much of the allelic variation is due to single nucleotide polymorphisms (SNPs), which include restriction fragment length polymorphisms (RFLPs).

Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences can comfortably provide positive individual identification with a panel of perhaps 10 to 1,000 primers that each yield a noncoding amplified sequence of 100 bases. If predicted coding sequences, such as those in SEQ ID NOS:2n−1, wherein n is an integer between 1 and 162 are used, a more appropriate number of primers for positive individual identification would be 500-2,000.

Predictive Medicine

The invention also pertains to the field of predictive medicine in which diagnostic assays, prognostic assays, pharmacogenomics, and monitoring clinical trials are used for prognostic (predictive) purposes to thereby treat an individual prophylactically. Accordingly, one aspect of the invention relates to diagnostic assays for determining NOVX protein and/or nucleic acid expression as well as NOVX activity, in the context of a biological sample (e.g., blood, serum, cells, tissue) to thereby determine whether an individual is afflicted with a disease or disorder, or is at risk of developing a disorder, associated with aberrant NOVX expression or activity. The disorders include metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, and the various dyslipidemias, metabolic disturbances associated with obesity, the metabolic syndrome X and wasting disorders associated with chronic diseases and various cancers. The invention also provides for prognostic (or predictive) assays for determining whether an individual is at risk of developing a disorder associated with NOVX protein, nucleic acid expression or activity. For example, mutations in an NOVX gene can be assayed in a biological sample. Such assays can be used for prognostic or predictive purpose to thereby prophylactically treat an individual prior to the onset of a disorder characterized by or associated with NOVX protein, nucleic acid expression, or biological activity.

Another aspect of the invention provides methods for determining NOVX protein, nucleic acid expression or activity in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent.)

Yet another aspect of the invention pertains to monitoring the influence of agents (e.g., drugs, compounds) on the expression or activity of NOVX in clinical trials.

These and other agents are described in further detail in the following sections.

Diagnostic Assays

An exemplary method for detecting the presence or absence of NOVX in a biological sample involves obtaining a biological sample from a test subject and contacting the biological sample with a compound or an agent capable of detecting NOVX protein or nucleic acid (e.g., mRNA, genomic DNA) that encodes NOVX protein such that the presence of NOVX is detected in the biological sample. An agent for detecting NOVX mRNA or genomic DNA is a labeled nucleic acid probe capable of hybridizing to NOVX mRNA or genomic DNA. The nucleic acid probe can be, for example, a full-length NOVX nucleic acid, such as the nucleic acid of SEQ ID NOS:2n−1, wherein n is an integer between 1 and 162, or a portion thereof, such as an oligonucleotide of at least 15, 30, 50, 100, 250 or 500 nucleotides in length and sufficient to specifically hybridize under stringent conditions to NOVX mRNA or genomic DNA. Other suitable probes for use in the diagnostic assays of the invention are described herein.

An agent for detecting NOVX protein is an antibody capable of binding to NOVX protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. That is, the detection method of the invention can be used to detect NOVX mRNA, protein, or genomic DNA in a biological sample in vitro as well as in vivo. For example, in vitro techniques for detection of NOVX mRNA include Northern hybridizations and in situ hybridizations. In vitro techniques for detection of NOVX protein include enzyme linked immunosorbent assays (ELISAs), Western blots, immunoprecipitations, and immunofluorescence. In vitro techniques for detection of NOVX genomic DNA include Southern hybridizations. Furthermore, in vivo techniques for detection of NOVX protein include introducing into a subject a labeled anti-NOVX antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a peripheral blood leukocyte sample isolated by conventional means from a subject.

In another embodiment, the methods further involve obtaining a control biological sample from a control subject, contacting the control sample with a compound or agent capable of detecting NOVX protein, mRNA, or genomic DNA, such that the presence of NOVX protein, mRNA or genomic DNA is detected in the biological sample, and comparing the presence of NOVX protein, mRNA or genomic DNA in the control sample with the presence of NOVX protein, mRNA or genomic DNA in the test sample.

The invention also encompasses kits for detecting the presence of NOVX in a biological sample. For example, the kit can comprise: a labeled compound or agent capable of detecting NOVX protein or mRNA in a biological sample; means for determining the amount of NOVX in the sample; and means for comparing the amount of NOVX in the sample with a standard. The compound or agent can be packaged in a suitable container. The kit can further comprise instructions for using the kit to detect NOVX protein or nucleic acid.

Prognostic Assays

The diagnostic methods described herein can furthermore be utilized to identify subjects having or at risk of developing a disease or disorder associated with aberrant NOVX expression or activity. For example, the assays described herein, such as the preceding diagnostic assays or the following assays, can be utilized to identify a subject having or at risk of developing a disorder associated with NOVX protein, nucleic acid expression or activity. Alternatively, the prognostic assays can be utilized to identify a subject having or at risk for developing a disease or disorder. Thus, the invention provides a method for identifying a disease or disorder associated with aberrant NOVX expression or activity in which a test sample is obtained from a subject and NOVX protein or nucleic acid (e.g., mRNA, genomic DNA) is detected, wherein the presence of NOVX protein or nucleic acid is diagnostic for a subject having or at risk of developing a disease or disorder associated with aberrant NOVX expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant NOVX expression or activity. For example, such methods can be used to determine whether a subject can be effectively treated with an agent for a disorder. Thus, the invention provides methods for determining whether a subject can be effectively treated with an agent for a disorder associated with aberrant NOVX expression or activity in which a test sample is obtained and NOVX protein or nucleic acid is detected (e.g., wherein the presence of NOVX protein or nucleic acid is diagnostic for a subject that can be administered the agent to treat a disorder associated with aberrant NOVX expression or activity).

The methods of the invention can also be used to detect genetic lesions in an NOVX gene, thereby determining if a subject with the lesioned gene is at risk for a disorder characterized by aberrant cell proliferation and/or differentiation. In various embodiments, the methods include detecting, in a sample of cells from the subject, the presence or absence of a genetic lesion characterized by at least one of an alteration affecting the integrity of a gene encoding an NOVX-protein, or the misexpression of the NOVX gene. For example, such genetic lesions can be detected by ascertaining the existence of at least one of: (i) a deletion of one or more nucleotides from an NOVX gene; (ii) an addition of one or more nucleotides to an NOVX gene; (iii) a substitution of one or more nucleotides of an NOVX gene, (iv) a chromosomal rearrangement of an NOVX gene; (v) an alteration in the level of a messenger RNA transcript of an NOVX gene, (vi) aberrant modification of an NOVX gene, such as of the methylation pattern of the genomic DNA, (vii) the presence of a non-wild-type splicing pattern of a messenger RNA transcript of an NOVX gene, (viii) a non-wild-type level of an NOVX protein, (ix) allelic loss of an NOVX gene, and (x) inappropriate post-translational modification of an NOVX protein. As described herein, there are a large number of assay techniques known in the art which can be used for detecting lesions in an NOVX gene. A preferred biological sample is a peripheral blood leukocyte sample isolated by conventional means from a subject. However, any biological sample containing nucleated cells may be used, including, for example, buccal mucosal cells.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran, et al., 1988. Science 241: 1077-1080; and Nakazawa, et al., 1994. Proc. Natl. Acad. Sci. USA 91: 360-364), the latter of which can be particularly useful for detecting point mutations in the NOVX-gene (see, Abravaya, et al., 1995. Nucl. Acids Res. 23: 675-682). This method can include the steps of collecting a sample of cells from a patient, isolating nucleic acid (e.g., genomic, mRNA or both) from the cells of the sample, contacting the nucleic acid sample with one or more primers that specifically hybridize to an NOVX gene under conditions such that hybridization and amplification of the NOVX gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (see, Guatelli, et al., 1990. Proc. Natl. Acad. Sci. USA 87: 1874-1878), transcriptional amplification system (see, Kwoh, et al., 1989. Proc. Natl. Acad. Sci. USA 86: 1173-1177); Qβ Replicase (see, Lizardi, et al, 1988. BioTechnology 6: 1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in an NOVX gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, e.g., U.S. Pat. No. 5,493,531) can be used to score for the presence of specific mutations by development or loss of a ribozyme cleavage site.

In other embodiments, genetic mutations in NOVX can be identified by hybridizing a sample and control nucleic acids, e.g., DNA or RNA, to high-density arrays containing hundreds or thousands of oligonucleotides probes. See, e.g., Cronin, et al., 1996. Human Mutation 7: 244-255; Kozal, et al., 1996. Nat. Med. 2: 753-759. For example, genetic mutations in NOVX can be identified in two dimensional arrays containing light-generated DNA probes as described in Cronin, et al., supra. Briefly, a first hybridization array of probes can be used to scan through long stretches of DNA in a sample and control to identify base changes between the sequences by making linear arrays of sequential overlapping probes. This step allows the identification of point mutations. This is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the NOVX gene and detect mutations by comparing the sequence of the sample NOVX with the corresponding wild-type (control) sequence. Examples of sequencing reactions include those based on techniques developed by Maxim and Gilbert, 1977. Proc. Natl. Acad. Sci. USA 74: 560 or Sanger, 1977. Proc. Natl. Acad. Sci. USA 74: 5463. It is also contemplated that any of a variety of automated sequencing procedures can be utilized when performing the diagnostic assays (see, e.g., Naeve, et al., 1995. Biotechniques 19: 448), including sequencing by mass spectrometry (see, e.g., PCT International Publication No. WO 94/16101; Cohen, et al., 1996. Adv. Chromatography 36: 127-162; and Griffin, et al., 1993. Appl. Biochem. Biotechnol. 38: 147-159).

Other methods for detecting mutations in the NOVX gene include methods in which protection from cleavage agents is used to detect mismatched bases in RNA/RNA or RNA/DNA heteroduplexes. See, e.g., Myers, et al., 1985. Science 230: 1242. In general, the art technique of “mismatch cleavage” starts by providing heteroduplexes of formed by hybridizing (labeled) RNA or DNA containing the wild-type NOVX sequence with potentially mutant RNA or DNA obtained from a tissue sample. The double-stranded duplexes are treated with an agent that cleaves single-stranded regions of the duplex such as which will exist due to basepair mismatches between the control and sample strands. For instance, RNA/DNA duplexes can be treated with Cleavage signal-1 protein and DNA/DNA hybrids treated with S₁ nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, e.g., Cotton, et al., 1988. Proc. Natl. Acad. Sci. USA 85: 4397; Saleeba, et al., 1992. Methods Enzymol. 217: 286-295. In an embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in NOVX cDNAs obtained from samples of cells. For example, the mutY enzyme of E. coli cleaves A at G/A mismatches and the thymidine DNA glycosylase from HeLa cells cleaves T at G/T mismatches. See, e.g., Hsu, et al., 1994. Carcinogenesis 15: 1657-1662. According to an exemplary embodiment, a probe based on an NOVX sequence, e.g., a wild-type NOVX sequence, is hybridized to a cDNA or other DNA product from a test cell(s). The duplex is treated with a DNA mismatch repair enzyme, and the cleavage products, if any, can be detected from electrophoresis protocols or the like. See, e.g., U.S. Pat. No. 5,459,039.

In other embodiments, alterations in electrophoretic mobility will be used to identify mutations in NOVX genes. For example, single strand conformation polymorphism (SSCP) may be used to detect differences in electrophoretic mobility between mutant and wild type nucleic acids. See, e.g., Orita, et al., 1989. Proc. Natl. Acad. Sci. USA: 86: 2766; Cotton, 1993. Mutat. Res. 285: 125-144; Hayashi, 1992. Genet. Anal. Tech. Appl. 9: 73-79. Single-stranded DNA fragments of sample and control NOVX nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In one embodiment, the subject method utilizes heteroduplex analysis to separate double stranded heteroduplex molecules on the basis of changes in electrophoretic mobility. See, e.g., Keen, et al., 1991. Trends Genet. 7: 5.

In yet another embodiment, the movement of mutant or wild-type fragments in polyacrylamide gels containing a gradient of denaturant is assayed using denaturing gradient gel electrophoresis (DGGE). See, e.g., Myers, et al., 1985. Nature 313: 495. When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA. See, e.g., Rosenbaum and Reissner, 1987. Biophys. Chem. 265: 12753.

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions that permit hybridization only if a perfect match is found. See, e.g., Saiki, et al., 1986. Nature 324: 163; Saiki, et al., 1989. Proc. Natl. Acad. Sci. USA 86: 6230. Such allele specific oligonucleotides are hybridized to PCR amplified target DNA or a number of different mutations when the oligonucleotides are attached to the hybridizing membrane and hybridized with labeled target DNA.

Alternatively, allele specific amplification technology that depends on selective PCR amplification may be used in conjunction with the instant invention. Oligonucleotides used as primers for specific amplification may carry the mutation of interest in the center of the molecule (so that amplification depends on differential hybridization; see, e.g., Gibbs, et al., 1989. Nucl. Acids Res. 17: 2437-2448) or at the extreme 3′-terminus of one primer where, under appropriate conditions, mismatch can prevent, or reduce polymerase extension (see, e.g., Prossner, 1993. Tibtech. 11: 238). In addition it may be desirable to introduce a novel restriction site in the region of the mutation to create cleavage-based detection. See, e.g., Gasparini, et al., 1992. Mol. Cell Probes 6: 1. It is anticipated that in certain embodiments amplification may also be performed using Taq ligase for amplification. See, e.g., Barany, 1991. Proc. Natl. Acad. Sci. USA 88: 189. In such cases, ligation will occur only if there is a perfect match at the 3′-terminus of the 5′ sequence, making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving an NOVX gene.

Furthermore, any cell type or tissue, preferably peripheral blood leukocytes, in which NOVX is expressed may be utilized in the prognostic assays described herein. However, any biological sample containing nucleated cells may be used, including, for example, buccal mucosal cells.

Pharmacogenomics

Agents, or modulators that have a stimulatory or inhibitory effect on NOVX activity (e.g., NOVX gene expression), as identified by a screening assay described herein can be administered to individuals to treat (prophylactically or therapeutically) disorders (The disorders include metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, and the various dyslipidemias, metabolic disturbances associated with obesity, the metabolic syndrome X and wasting disorders associated with chronic diseases and various cancers.) In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of NOVX protein, expression of NOVX nucleic acid, or mutation content of NOVX genes in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See e.g., Eichelbaum, 1996. Clin. Exp. Pharmacol. Physiol., 23: 983-985; Linder, 1997. Clin. Chem., 43: 254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare defects or as polymorphisms. For example, glucose-6-phosphate dehydrogenase (G6PD) deficiency is a common inherited enzymopathy in which the main clinical complication is hemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome NEUROPEPTIDE Y/PEPTIDE YY RECEPTOR enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. At the other extreme are the so called ultra-rapid metabolizers who do not respond to standard doses. Recently, the molecular basis of ultra-rapid metabolism has been identified to be due to CYP2D6 gene amplification.

Thus, the activity of NOVX protein, expression of NOVX nucleic acid, or mutation content of NOVX genes in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with an NOVX modulator, such as a modulator identified by one of the exemplary screening assays described herein.

Monitoring of Effects During Clinical Trials

Monitoring the influence of agents (e.g., drugs, compounds) on the expression or activity of NOVX (e.g., the ability to modulate aberrant cell proliferation and/or differentiation) can be applied not only in basic drug screening, but also in clinical trials. For example, the effectiveness of an agent determined by a screening assay as described herein to increase NOVX gene expression, protein levels, or upregulate NOVX activity, can be monitored in clinical trails of subjects exhibiting decreased NOVX gene expression, protein levels, or downregulated NOVX activity. Alternatively, the effectiveness of an agent determined by a screening assay to decrease NOVX gene expression, protein levels, or downregulate NOVX activity, can be monitored in clinical trails of subjects exhibiting increased NOVX gene expression, protein levels, or upregulated NOVX activity. In such clinical trials, the expression or activity of NOVX and, preferably, other genes that have been implicated in, for example, a cellular proliferation or immune disorder can be used as a “read out” or markers of the immune responsiveness of a particular cell.

By way of example, and not of limitation, genes, including NOVX, that are modulated in cells by treatment with an agent (e.g., compound, drug or small molecule) that modulates NOVX activity (e.g., identified in a screening assay as described herein) can be identified. Thus, to study the effect of agents on cellular proliferation disorders, for example, in a clinical trial, cells can be isolated and RNA prepared and analyzed for the levels of expression of NOVX and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of NOVX or other genes. In this manner, the gene expression pattern can serve as a marker, indicative of the physiological response of the cells to the agent. Accordingly, this response state may be determined before, and at various points during, treatment of the individual with the agent.

In one embodiment, the invention provides a method for monitoring the effectiveness of treatment of a subject with an agent (e.g., an agonist, antagonist, protein, peptide, peptidomimetic, nucleic acid, small molecule, or other drug candidate identified by the screening assays described herein) comprising the steps of (i) obtaining a pre-administration sample from a subject prior to administration of the agent; (ii) detecting the level of expression of an NOVX protein, mRNA, or genomic DNA in the preadministration sample; (iii) obtaining one or more post-administration samples from the subject; (iv) detecting the level of expression or activity of the NOVX protein, mRNA, or genomic DNA in the post-administration samples; (v) comparing the level of expression or activity of the NOVX protein, mRNA, or genomic DNA in the pre-administration sample with the NOVX protein, mRNA, or genomic DNA in the post administration sample or samples; and (vi) altering the administration of the agent to the subject accordingly. For example, increased administration of the agent may be desirable to increase the expression or activity of NOVX to higher levels than detected, i.e., to increase the effectiveness of the agent. Alternatively, decreased administration of the agent may be desirable to decrease expression or activity of NOVX to lower levels than detected, i.e., to decrease the effectiveness of the agent.

Methods of Treatment

The invention provides for both prophylactic and therapeutic methods of treating a subject at risk of (or susceptible to) a disorder or having a disorder associated with aberrant NOVX expression or activity. The disorders include cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, transplantation, adrenoleukodystrophy, congenital adrenal hyperplasia, prostate cancer, neoplasm; adenocarcinoma, lymphoma, uterus cancer, fertility, hemophilia, hypercoagulation, idiopathic thrombocytopenic purpura, immunodeficiencies, graft versus host disease, AIDS, bronchial asthma, Crohn's disease; multiple sclerosis, treatment of Albright Hereditary Ostoeodystrophy, and other diseases, disorders and conditions of the like.

These methods of treatment will be discussed more fully, below.

Disease and Disorders

Diseases and disorders that are characterized by increased (relative to a subject not suffering from the disease or disorder) levels or biological activity may be treated with Therapeutics that antagonize (i.e., reduce or inhibit) activity. Therapeutics that antagonize activity may be administered in a therapeutic or prophylactic manner. Therapeutics that may be utilized include, but are not limited to: (i) an aforementioned peptide, or analogs, derivatives, fragments or homologs thereof; (ii) antibodies to an aforementioned peptide; (iii) nucleic acids encoding an aforementioned peptide; (iv) administration of antisense nucleic acid and nucleic acids that are “dysfunctional” (i.e., due to a heterologous insertion within the coding sequences of coding sequences to an aforementioned peptide) that are utilized to “knockout” endogenous function of an aforementioned peptide by homologous recombination (see, e.g., Capecchi, 1989. Science 244: 1288-1292); or (v) modulators (i.e., inhibitors, (see, e.g., Capecchi, 1989. Science 244: 1288-1292); or (v) modulators (i.e., inhibitors, specific to a peptide of the invention) that alter the interaction between an aforementioned peptide and its binding partner.

Diseases and disorders that are characterized by decreased (relative to a subject not suffering from the disease or disorder) levels or biological activity may be treated with Therapeutics that increase (i.e., are agonists to) activity. Therapeutics that upregulate activity may be administered in a therapeutic or prophylactic manner. Therapeutics that may be utilized include, but are not limited to, an aforementioned peptide, or analogs, derivatives, fragments or homologs thereof, or an agonist that increases bioavailability.

Increased or decreased levels can be readily detected by quantifying peptide and/or RNA, by obtaining a patient tissue sample (e.g., from biopsy tissue) and assaying it in vitro for RNA or peptide levels, structure and/or activity of the expressed peptides (or mRNAs of an aforementioned peptide). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, and the like).

Prophylactic Methods

In one aspect, the invention provides a method for preventing, in a subject, a disease or condition associated with an aberrant NOVX expression or activity, by administering to the subject an agent that modulates NOVX expression or at least one NOVX activity. Subjects at risk for a disease that is caused or contributed to by aberrant NOVX expression or activity can be identified by, for example, any or a combination of diagnostic or prognostic assays as described herein. Administration of a prophylactic agent can occur prior to the manifestation of symptoms characteristic of the NOVX aberrancy, such that a disease or disorder is prevented or, alternatively, delayed in its progression. Depending upon the type of NOVX aberrancy, for example, an NOVX agonist or NOVX antagonist agent can be used for treating the subject. The appropriate agent can be determined based on screening assays described herein. The prophylactic methods of the invention are further discussed in the following subsections.

Therapeutic Methods

Another aspect of the invention pertains to methods of modulating NOVX expression or activity for therapeutic purposes. The modulatory method of the invention involves contacting a cell with an agent that modulates one or more of the activities of NOVX protein activity associated with the cell. An agent that modulates NOVX protein activity can be an agent as described herein, such as a nucleic acid or a protein, a naturally-occurring cognate ligand of an NOVX protein, a peptide, an NOVX peptidomimetic, or other small molecule. In one embodiment, the agent stimulates one or more NOVX protein activity. Examples of such stimulatory agents include active NOVX protein and a nucleic acid molecule encoding NOVX that has been introduced into the cell. In another embodiment, the agent inhibits one or more NOVX protein activity. Examples of such inhibitory agents include antisense NOVX nucleic acid molecules and anti-NOVX antibodies. These modulatory methods can be performed in vitro (e.g., by culturing the cell with the agent) or, alternatively, in vivo (e.g., by administering the agent to a subject). As such, the invention provides methods of treating an individual afflicted with a disease or disorder characterized by aberrant expression or activity of an NOVX protein or nucleic acid molecule. In one embodiment, the method involves administering an agent (e.g., an agent identified by a screening assay described herein), or combination of agents that modulates (e.g., up-regulates or down-regulates) NOVX expression or activity. In another embodiment, the method involves administering an NOVX protein or nucleic acid molecule as therapy to compensate for reduced or aberrant NOVX expression or activity.

Stimulation of NOVX activity is desirable in situations in which NOVX is abnormally downregulated and/or in which increased NOVX activity is likely to have a beneficial effect. One example of such a situation is where a subject has a disorder characterized by aberrant cell proliferation and/or differentiation (e.g., cancer or immune associated disorders). Another example of such a situation is where the subject has a gestational disease (e.g., preclampsia).

Determination of the Biological Effect of the Therapeutic

In various embodiments of the invention, suitable in vitro or in vivo assays are performed to determine the effect of a specific Therapeutic and whether its administration is indicated for treatment of the affected tissue.

In various specific embodiments, in vitro assays may be performed with representative cells of the type(s) involved in the patient's disorder, to determine if a given Therapeutic exerts the desired effect upon the cell type(s). Compounds for use in therapy may be tested in suitable animal model systems including, but not limited to rats, mice, chicken, cows, monkeys, rabbits, and the like, prior to testing in human subjects. Similarly, for in vivo testing, any of the animal model system known in the art may be used prior to administration to human subjects.

Prophylactic and Therapeutic Uses of the Compositions of the Invention

The NOVX nucleic acids and proteins of the invention are useful in potential prophylactic and therapeutic applications implicated in a variety of disorders including, but not limited to: metabolic disorders, diabetes, obesity, infectious disease, anorexia, cancer-associated cancer, neurodegenerative disorders, Alzheimer's Disease, Parkinson's Disorder, immune disorders, hematopoietic disorders, and the various dyslipidemias, metabolic disturbances associated with obesity, the metabolic syndrome X and wasting disorders associated with chronic diseases and various cancers.

As an example, a cDNA encoding the NOVX protein of the invention may be useful in gene therapy, and the protein may be useful when administered to a subject in need thereof. By way of non-limiting example, the compositions of the invention will have efficacy for treatment of patients suffering from: metabolic disorders, diabetes, obesity, infectious disease, anorexia, cancer-associated cachexia, cancer, neurodegenerative disorders, Alzheimer's Disease, Parkinson's Disorder, immune disorders, hematopoietic disorders, and the various dyslipidemias.

Both the novel nucleic acid encoding the NOVX protein, and the NOVX protein of the invention, or fragments thereof, may also be useful in diagnostic applications, wherein the presence or amount of the nucleic acid or the protein are to be assessed. A further use could be as an anti-bacterial molecule (i.e., some peptides have been found to possess anti-bacterial properties). These materials are further useful in the generation of antibodies, which immunospecifically-bind to the novel substances of the invention for use in therapeutic or diagnostic methods.

The invention will be further described in the following examples, which do not limit the scope of the invention described in the claims.

### EXAMPLES

### Example 1

### Identification of NOVX Clones

The novel NOVX target sequences identified in the present invention were subjected to the exon linking process to confirm the sequence. PCR primers were designed by starting at the most upstream sequence available, for the forward primer, and at the most downstream sequence available for the reverse primer. Table 100A shows the sequences of the PCR primers used for obtaining different clones. In each case, the sequence was examined, walking inward from the respective termini toward the coding sequence, until a suitable sequence that is either unique or highly selective was encountered, or, in the case of the reverse primer, until the stop codon was reached. Such primers were designed based on in silico predictions for the full length cDNA, part (one or more exons) of the DNA or protein sequence of the target sequence, or by translated homology of the predicted exons to closely related human sequences from other species. These primers were then employed in PCR amplification based on the following pool of human cDNAs: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea, uterus. Usually the resulting amplicons were gel purified, cloned and sequenced to high redundancy. The PCR product derived from exon linking was cloned into the pCR2.1 vector from Invitrogen. The resulting bacterial clone has an insert covering the entire open reading frame cloned into the pCR2.1 vector. Table 100B shows a list of these bacterial clones. The resulting sequences from all clones were assembled with themselves, with other fragments in CuraGen Corporation's database and with public ESTs. Fragments and ESTs were included as components for an assembly when the extent of their identity with another component of the assembly was at least 95% over 50 bp. In addition, sequence traces were evaluated manually and edited for corrections if appropriate. These procedures provide the sequence reported herein.

TABLE 100A
PCR Primers for Exon Linking

| | | SEQ | | SEQ |
|---|---|---|---|---|
| NOVX | | ID | | ID |
| Clone | Primer 1 (5′-3′) | NO | Primer 2 (5′-3′) | NO |
| NOV1d | TGACTATGGCCTGGAGTTTCCGT | 890 | TTATTCCAGAGATCCTTGGCAGAAGC | 891 |
| NOV3c | ACCCTGGCCCAGCTCTGAGT | 892 | GAGCTCAGACGTACTCTCGGCCAG | 893 |
| NOV4c | ACCTGTCGCAATGCCTTTAATCTTTAG | 894 | ATCTCTGGCCTTGTCGGAOTCTTAC | 895 |
| NOV5a | GTTTCCCCACCCCGCAGA | 896 | GAGAGGATCAAAGAACCAGACAGGAG | 897 |
| NOV5b | ACGGCGATGACCCCCCAG | 898 | GGGGGAGGGGCTCAAACAAGA | 899 |
| NOV5c | ACGGCGATGACCCCCCAG | 900 | GGGGGAGGGGCTCAAACAAGA | 901 |
| NOV5d | ACGGCGATGACCCCCCAG | 902 | GGGGGAGGGGCTCAAACAAGA | 903 |
| NOV5e | ACGGCGATGACCCCCCAG | 904 | GGGGGAGGGGCTCAAACAAGA | 905 |
| NOV6 | GTCGAACCGGGGGACCTG | 906 | TGGGGAAGOTGCTCAGCCC | 907 |
| NOV7a | CAGCCAAACCCACCTCCACCAT | 908 | TTTGGCTGGCTTATATAGCGAGCTCCT | 909 |
| NOV7b | CATTGCCAATTCTAAATCCATCATTTG | 910 | TCTTCTAAAGCACAAATAACACCTCCA | 911 |
| NOV7c | ATGCCCGAGGCCGCGGAG | 912 | AGCTTCATTTCATTCTTTTTGCAACATCTG | 913 |
| NOV8 | GGGGACCATGGGGAGCGATC | 914 | CGAGAGGTTTTCTAGGCAGATTTGGAGC | 915 |
| NOV15b | ATGGCAGCAGAAAACCATTCTTTT | 916 | TCTGTTCATAAGAATGTTCTTTTCCCTAGC | 917 |
| NOV18b | AGATGGATGGAACCAATGGCAG | 918 | AGTGGCCCTGCATGOAAGTGA | 919 |
| NOV22c | CACTCCAACAGTTAATGCTTCCCTTG | 920 | GATGGTCTCGATCTCCTGACCTCTT | 921 |
| NOV24b | ATGGCCGAGGCCCCGGAG | 922 | AGCTTCATTTCATTCTTTTTGCAACATCTG | 923 |
| NOV26b | GTTCCTGCTGTCTGGACTTTTTCTGT | 924 | TTTTTGGAGAAAGCTGCAAAAGTTTAT | 925 |
| NOV27b | CTCAGTCCCTCOGGCTCATACCTA | 926 | GGACTCAGAGCTCCTGCCTTTCTGT | 927 |
| NOV27c | CTCAGTCCCTCGGGCTCATACCTA | 928 | CTCAGAGCTCCTGCCTTTCTGTCC | 929 |
| NOV29b | GTAGCCACAAGACCGGGTCCG | 930 | CCCTGGCCTCTTGGAACTGCTTGAT | 931 |
| NOV32 | CTTGGAGCCTGCAGGTCCTGGAC | 932 | AGGAGCATCCTTCATCCCACTAGAGGT | 933 |
| NOV33 | GCTGGATTGGGTGATCTCTCAGAGC | 934 | CTCTTACTCCTGGCAAGCCCTGC | 935 |
| NOV36b | ATGCAAGAAACAATTTTTTTTTTTGAGA | 936 | TTTTACCCATTCACCAGTTTAAGGACG | 937 |
| | CG | | | |
| NOV38 | CAATGACCTCCTCATTGCTTCTGG | 938 | CAAAGCCCCAOGTCCTCTTGCTAG | 939 |
| NOV39a | CCATCCGAGGCTCCTGAACC | 940 | CAAAGCCCCAGGTCCTCTTGC | 941 |
| NOV39b | GTTCTTGAACCAGGGCCATTCAC | 942 | CCTGGGAAACTTCATCTTGGTCTCTT | 943 |
| NOV42b | ATGGACGGTGAGOCAGTCC | 944 | CCACACCCTGGCCCATG | 945 |
| NOV42c | ATGGACGGTGAGOCAGTCC | 946 | CCACACCCTGGCCCATG | 947 |
| NOV42d | CTGGAGGATGAAGGAAGCAGAGATG | 948 | CCAGAGAACAAGCAGAGCTCAGAGG | 949 |
| NOV46b | GTTTCTGAGCATGGATCCAACCA | 950 | AAAGGGCAGAGGCTCTTCCTCAC | 951 |
| NOV48b | GCVACCCTCTCTGCTGGCTACCTAAC | 952 | TTGATTTTCACCACCTCCATTTGTTCT | 953 |
| NOV50b | AAGATGTCGCAGCCCAAGAAAAG | 954 | TGCTTTGGGACGTAGCTGGGA | 955 |
| NOV51 | CTGGCCTAATGAATGTCTCTGAGC | 956 | TGATCATGGAGGAATAATCTAATATGCCTTA | 957 |
| | | | G | |
| NOV52 | TCCCTGATGTCCACCTCTGGCT | 958 | ATAGACTAACTCCACCCACAGGCCTCT | 959 |
| NOV56b | ATGGCGAAGATTGAGAAAAACCCT | 960 | CTCTCAGATCTCCAGGCAGAAGTTCAG | 961 |
| NOV58b | AGTCTTGCCTTCTTTTGAGCCTAAGTC | 962 | CACATTCAACATATCTGAGGCTGTGG | 963 |
| NOV60a | CGAATTGGCTTCCGAGTGAAAATC | 964 | TTATTTAAAGGTCAAGGCCTCGAAGTG | 965 |
| NOV61 | CAGCTGTGCCCTCATCCTTGTGCCTGCT | 966 | CTCGAGCTTGGTCACTGTGATTCCCACCGTG | 967 |
| | ACGTCC | | ATATGGTCTGCC | |
| NOV63 | GGATCCAGCTACCCCATCTGGTCGCTGA | 968 | GTCGACGCCCTTGCAGGTGTAGACCTCCTCA | 969 |
| | CGGGCAGC | | CG | |
| NOV68 | GGCACGCTCCCTCTGGCT | 970 | TTCACTGTGGGGGCCTGG | 971 |
| NOV69b | ATGGAAAAAGCATTGAAAATTGACACA | 972 | TTAAAACACCATAGTTAACCCAAACTCAGTA | 973 |
| | | | GTG | |
| NOV70a | TATGCTGTCTATGCTGAATCTTCTAATC | 974 | TTTAAGAATCTTCAATATTGGCCCCCAC | 975 |
| | TTGTCT | | | |
| NOV76b | ACCATTACATCATCGTCGCAAATTAAA | 976 | GAAGTCACAACTGTCTTTCTTCTCAGGA | 977 |
| NOV81b | GTCATGCGCTGCCCCAAGT | 978 | CCAATGAGAGTCACCACTGGAGC | 979 |
| NOV87b | TCTCTCATGGCCCCCAAAGAC | 980 | AGTCAGTGCGGCGGGAAGA | 981 |
| NOV96a | GGTGCTCTCAGCGTTCTTCCAGTC | 982 | CTAGTGCTTCTGTTACAAGGTCCTGGG | 983 |
| NOV96d | AGCTGGATTGACAACTTTGTAATGCAG | 984 | CTCAGTCGTGCTGCTAGTAGGGGT | 985 |
| NOV96e | TGAAGCTCACCAGGAGGAAGAAG | 986 | CTCAGTCGTGCTGCTAGTAGGGGT | 987 |
| NOV96f | TCCCATGACCTGCCACTTCC | 988 | CGCTACCTGCAGCCGCA | 989 |
| NOV98 | CCGGCCCCGTGTGTGGCA | 990 | GGGGCTCTGGTGCCAGCTCATG | 991 |
| NOV99 | CACCCAAACCCACCTCCACCAT | 992 | TTTGGCTGGCTTATATACCCAGCTCCT | 993 |

Physical clone: Exons were predicted by homology and the intron/exon boundaries were determined using standard genetic rules. Exons were further selected and refined by means of similarity determination using multiple BLAST (for example, tBlastN, BlastX, and BlastN) searches, and, in some instances, GeneScan and Grail. Expressed sequences from both public and proprietary databases were also added when available to further define and complete the gene sequence. The DNA sequence was then manually corrected for apparent inconsistencies thereby obtaining the sequences encoding the full-length protein.

TABLE 100B
Physical Clones for PCR products

| NOVX Clone | Bacterial Clone |
|---|---|
| NOV1a | Genomic clone: AC080137 |
| NOV1b | Genomic clone: GMAC036188 |
| NOV1d | Physical clone: GMAC080137_A.698589.M14 |
| NOV2 | Physical clone: AC023194 |
| NOV3a | Genomic clone: AL136383 |
| NOV3c | Physical clone: GMAL136383_A.698589.C6 |
| NOV4a | Genomic clone: AC046164 |
| NOV4c | Physical clone: GMAC046164_A.698589.A2 |
| NOV5c | Physical clone: 155643499 |
| NOV5d | Physical clone: 155643499 |
| NOV5e | Physical clone: 127939::153729589.698590.P9 |
| NOV7a | Physical clone: 153634912 |
| NOV7b | Physical clone: 127994::138896306.698587.E16 |
| NOV7c | Physical clone: 127557::CG564550-01.698587.F19 |
| NOV9a | Physical clone: 152568459, AC005961.1, |
| | AC068256.2, AI308124, |
| | AI307658 |
| NOV10 | Physical clone: 126218::CG55964-01.698509.G10 |
| NOV13b | Physical clone: 126388::CG56021-01.698539.G15 |
| NOV15b | Physical clone: 126694::CG56065-01.698561.C17 |
| NOV16a | Physical clone: 126696::CG56067-01.698561.A11 |
| NOV16b | Physical clone: 128769::GMAC084434_A.698655.G14 |
| NOV17b | Physical clone: 128198::GMba430i15_A.698589.F10 |
| NOV17d | Physical clone: 105889::sggc_draft_{—} |
| | ba430i15_20000823.698368.G10 |
| NOV18b | Physical clone: 128205::GMba430i15_D.698590.E4 |
| NOV19b | Physical clone: 128207::GMba430i15_E.698590.C4 |
| NOV21a | Physical clone: 153485226, 145329583 |
| NOV21b | Physical clone: 50119::86375666.244185.F7 |
| NOV22a | Genomic clone: AC011522.6 |
| NOV22c | Physical clone: 161710747.698893.K3 |
| NOV23a | Physical clone: 140117553 146712128 |
| | 146712112 148412737 |
| NOV23c | Physical clone: 140117553 |
| NOV24b | Physical clone: 127557::CG564550-01.698591.N11 |
| NOV25 | Physical clone: 151537975 and 128978463 |
| NOV26a | Physical clone: 127998335 |
| NOV26b | Physical clone: 127561::CG56461-01.698589.G7 |
| NOV27a | Physical clone: 111787393, HSAJ9617 |
| NOV27b | Physical clone: 111787393_EXT.698587.K18 |
| NOV27c | Physical clone: 167695055 170842341 170842333 |
| NOV28 | Physical clone: AC004832 |
| NOV29a | Physical clone: AC004832 |
| NOV29b | Physical clone: 112824::COR100399281.698230.B12 |
| NOV29c | Physical clone: AC004832 |
| NOV30 | Genomic clone: AC004832 |
| NOV31 | Genomic clone: 94329210 |
| NOV33 | Physical clone: 153778095, 138978176, |
| | 146713055, 105100551, |
| | 153777948 |
| NOV34 | Physical clone: 125858::GMAC026083_E.698508.J15 |
| NOV35 | Physical clone: 114740::AC011711.698329.I10 |
| NOV36a | Physical clone: 152568436, AL132780 |
| NOV36b | Physical clone: 152568436 134511756 |
| NOV37 | Physical clone: 151557368, 138195002, |
| | 152762569, 152768078 |
| NOV38 | Physical clone: 107207::AC061707.698315.F14 |
| NOV39a | Physical clone: 127119::AC061707.698564.H20 |
| NOV39b | Physical clone: 128110::ADENOSINE_{—} |
| | A3_RECEPTOR.698657.H22. |
| NOV40 | Physical clone: AC068471, AC068471, |
| | AV655524, T67857 |
| NOV41a | Physical clone: AC007278, AW242630.1 xn01f05.x1 |
| NOV42a | Physical clone: AC007395, 153103275, |
| | 153103263, 153103260 |
| NOV42b | Physical clone: 153103275 153103263 |
| | 153103260 153103539 |
| | 153103266 152189065 |
| NOV42c | Physical clone: 54701683_EXT.698433.J23 |
| NOV42d | Physical clone: AC007395_A.698587.M17 |
| NOV43 | Genomic clone: AC021773 |
| NOV44 | Physical clone: AC023654, 78743598 |
| NOV45 | Genomic clone: AC023078 |
| NOV46a | Genomic clone: AC023078 |
| NOV46b | Physical clone: 128292::AC023078_A.698657.G13 |
| NOV46c | Genomic clone: AC023654 |
| NOV46d | Physical clone: 151667972 |
| NOV47 | Genomic clone: AF152363 |
| NOV48a | Genomic clone: AC012510.5 |
| NOV48b | Physical clone: 128669::AC012510_5_final.698656.J20 |
| NOV49 | Genomic clone: AC011492 |
| NOV50a | Physical clone: 153778754, 122656699 |
| NOV50b | Physical clone: AK001421_A.698657.J10 |
| NOV51 | Physical clone: 126131::CG55922-01.698509.O9 |
| NOV52 | Physical clone: 153623113, 152186811, 148441423, |
| | 148441418, 152186815, 152209564, |
| | 126066491, 129293170, 126630256, |
| | 124459512 |
| NOV53 | Physical clone: 151222559 |
| NOV54a | Physical clone: 153512063 |
| NOV55 | Genomic clone: AL138816.12, AL158192.12 |
| NOV56a | Genomic clone: AC019100.4 |
| NOV57 | Physical clone: 50222151, 150222148 |
| NOV54b | Physical clone: 164698940 |
| NOV56b | Physical clone: 55048::nh0443k08_A.698002.M9 |

### Example 2

### Quantitive Expression Analysis of Clones in Various Cells and Tissues

The quantitative expression of various clones was assessed using microtiter plates containing RNA samples from a variety of normal and pathology-derived cells, cell lines and tissues using real time quantitative PCR (RTQ PCR). RTQ PCR was performed on an Applied Biosystems ABI PRISM® 7700 or an ABI PRISM® 7900 HT Sequence Detection System. Various collections of samples are assembled on the plates, and referred to as Panel 1 (containing normal tissues and cancer cell lines), Panel 2 (containing samples derived from tissues from normal and cancer sources), Panel 3 (containing cancer cell lines), Panel 4 (containing cells and cell lines from normal tissues and cells related to inflammatory conditions), Panel 5D/5I (containing human tissues and cell lines with an emphasis on metabolic diseases), AI_comprehensive_panel (containing normal tissue and samples from autoimmune diseases), Panel CNSD.01 (containing central nervous system samples from normal and diseased brains) and CNS_neurodegeneration panel (containing samples from normal and Alzheimer's diseased brains).

RNA integrity from all samples is controlled for quality by visual assessment of agarose gel electropherograms using 28S and 18S ribosomal RNA staining intensity ratio as a guide (2:1 to 2.5:1 28s: 18s) and the absence of low molecular weight RNAs that would be indicative of degradation products. Samples are controlled against genomic DNA contamination by RTQ PCR reactions run in the absence of reverse transcriptase using probe and primer sets designed to amplify across the span of a single exon.

First, the RNA samples were normalized to reference nucleic acids such as constitutively expressed genes (for example, β-actin and GAPDH). Normalized RNA (5 ul) was converted to cDNA and analyzed by RTQ-PCR using One Step RT-PCR Master Mix Reagents (Applied Biosystems; Catalog No. 4309169) and gene-specific primers according to the manufacturer's instructions.

In other cases, non-normalized RNA samples were converted to single strand cDNA (sscDNA) using Superscript II (Invitrogen Corporation; Catalog No. 18064-147) and random hexamers according to the manufacturer's instructions. Reactions containing up to 10 μg of total RNA were performed in a volume of 20 μl and incubated for 60 minutes at 42° C. This reaction can be scaled up to 50 μg of total RNA in a final volume of 100 μl. sscDNA samples are then normalized to reference nucleic acids as described previously, using 1× TaqMan® Universal Master mix (Applied Biosystems; catalog No. 4324020), following the manufacturer's instructions.

Probes and primers were designed for each assay according to Applied Biosystems Primer Express Software package (version I for Apple Computer's Macintosh Power PC) or a similar algorithm using the target sequence as input. Default settings were used for reaction conditions and the following parameters were set before selecting primers: primer concentration=250 nM, primer melting temperature (Tm) range=58°-60° C., primer optimal Tm=59° C., maximum primer difference=2° C., probe does not have 5′G, probe Tm must be 10° C. greater than primer Tm, amplicon size 75 bp to 100 bp. The probes and primers selected (see below) were synthesized by Synthegen (Houston, Tex., USA). Probes were double purified by HPLC to remove uncoupled dye and evaluated by mass spectroscopy to verify coupling of reporter and quencher dyes to the 5′ and 3′ ends of the probe, respectively. Their final concentrations were: forward and reverse primers, 900 nM each, and probe, 200 nM.

PCR conditions: When working with RNA samples, normalized RNA from each tissue and each cell line was spotted in each well of either a 96 well or a 384-well PCR plate (Applied Biosystems). PCR cocktails included either a single gene specific probe and primers set, or two multiplexed probe and primers sets (a set specific for the target clone and another gene-specific set multiplexed with the target probe). PCR reactions were set up using TaqMan® One-Step RT-PCR Master Mix (Applied Biosystems, Catalog No. 4313803) following manufacturer's instructions. Reverse transcription was performed at 48° C. for 30 minutes followed by amplification/PCR cycles as follows: 95° C. 10 min, then 40 cycles of 95° C. for 15 seconds, 60° C. for 1 minute. Results were recorded as CT values (cycle at which a given sample crosses a threshold level of fluorescence) using a log scale, with the difference in RNA concentration between a given sample and the sample with the lowest CT value being represented as 2 to the power of delta CT. The percent relative expression is then obtained by taking the reciprocal of this RNA difference and multiplying by 100.

When working with sscDNA samples, normalized sscDNA was used as described previously for RNA samples. PCR reactions containing one or two sets of probe and primers were set up as described previously, using 1× TaqMan® Universal Master mix (Applied Biosystems; catalog No. 4324020), following the manufacturer's instructions. PCR amplification was performed as follows: 95° C. 10 min, then 40 cycles of 95° C. for 15 seconds, 60° C. for 1 minute. Results were analyzed and processed as described previously.

Panels 1, 1.1, 1.2, and 1.3D

The plates for Panels 1, 1.1, 1.2 and 1.3D include 2 control wells (genomic DNA control and chemistry control) and 94 wells containing cDNA from various samples. The samples in these panels are broken into 2 classes: samples derived from cultured cell lines and samples derived from primary normal tissues. The cell lines are derived from cancers of the following types: lung cancer, breast cancer, melanoma, colon cancer, prostate cancer, CNS cancer, squamous cell carcinoma, ovarian cancer, liver cancer, renal cancer, gastric cancer and pancreatic cancer. Cell lines used in these panels are widely available through the American Type Culture Collection (ATCC), a repository for cultured cell lines, and were cultured using the conditions recommended by the ATCC. The normal tissues found on these panels are comprised of samples derived from all major organ systems from single adult individuals or fetuses. These samples are derived from the following organs: adult skeletal muscle, fetal skeletal muscle, adult heart, fetal heart, adult kidney, fetal kidney, adult liver, fetal liver, adult lung, fetal lung, various regions of the brain, the spleen, bone marrow, lymph node, pancreas, salivary gland, pituitary gland, adrenal gland, spinal cord, thymus, stomach, small intestine, colon, bladder, trachea, breast, ovary, uterus, placenta, prostate, testis and adipose.

In the results for Panels 1, 1.1, 1.2 and 1.3D, the following abbreviations are used:

ca.=carcinoma,

*=established from metastasis,

met=metastasis,

s cell var=small cell variant,

non-s=non-sm=non-small,

squam=squamous,

pl. eff=pl effusion=pleural effusion,

glio=glioma,

astro=astrocytoma, and

neuro=neuroblastoma.

General_screening_panel_v1.4

The plates for Panel 1.4 include 2 control wells (genomic DNA control and chemistry control) and 94 wells containing cDNA from various samples. The samples in Panel 1.4 are broken into 2 classes: samples derived from cultured cell lines and samples derived from primary normal tissues. The cell lines are derived from cancers of the following types: lung cancer, breast cancer, melanoma, colon cancer, prostate cancer, CNS cancer, squamous cell carcinoma, ovarian cancer, liver cancer, renal cancer, gastric cancer and pancreatic cancer. Cell lines used in Panel 1.4 are widely available through the American Type Culture Collection (ATCC), a repository for cultured cell lines, and were cultured using the conditions recommended by the ATCC. The normal tissues found on Panel 1.4 are comprised of pools of samples derived from all major organ systems from 2 to 5 different adult individuals or fetuses. These samples are derived from the following organs: adult skeletal muscle, fetal skeletal muscle, adult heart, fetal heart, adult kidney, fetal kidney, adult liver, fetal liver, adult lung, fetal lung, various regions of the brain, the spleen, bone marrow, lymph node, pancreas, salivary gland, pituitary gland, adrenal gland, spinal cord, thymus, stomach, small intestine, colon, bladder, trachea, breast, ovary, uterus, placenta, prostate, testis and adipose. Abbreviations are as described for Panels 1, 1.1, 1.2, and 1.3D.

Panels 2D and 2.2

The plates for Panels 2D and 2.2 generally include 2 control wells and 94 test samples composed of RNA or cDNA isolated from human tissue procured by surgeons working in close cooperation with the National Cancer Institute's Cooperative Human Tissue Network (CHTN) or the National Disease Research Initiative (NDRI). The tissues are derived from human malignancies and in cases where indicated many malignant tissues have “matched margins” obtained from noncancerous tissue just adjacent to the tumor. These are termed normal adjacent tissues and are denoted “NAT” in the results below. The tumor tissue and the “matched margins” are evaluated by two independent pathologists (the surgical pathologists and again by a pathologist at NDRI or CHTN). This analysis provides a gross histopathological assessment of tumor differentiation grade. Moreover, most samples include the original surgical pathology report that provides information regarding the clinical stage of the patient. These matched margins are taken from the tissue surrounding (i.e. immediately proximal) to the zone of surgery (designated “NAT”, for normal adjacent tissue, in Table RR). In addition, RNA and cDNA samples were obtained from various human tissues derived from autopsies performed on elderly people or sudden death victims (accidents, etc.). These tissues were ascertained to be free of disease and were purchased from various commercial sources such as Clontech (Palo Alto, Calif.), Research Genetics, and Invitrogen.

Panel 3D

The plates of Panel 3D are comprised of 94 cDNA samples and two control samples. Specifically, 92 of these samples are derived from cultured human cancer cell lines, 2 samples of human primary cerebellar tissue and 2 controls. The human cell lines are generally obtained from ATCC (American Type Culture Collection), NCI or the German tumor cell bank and fall into the following tissue groups: Squamous cell carcinoma of the tongue, breast cancer, prostate cancer, melanoma, epidermoid carcinoma, sarcomas, bladder carcinomas, pancreatic cancers, kidney cancers, leukemias/lymphomas, ovarian/uterine/cervical, gastric, colon, lung and CNS cancer cell lines. In addition, there are two independent samples of cerebellum. These cells are all cultured under standard recommended conditions and RNA extracted using the standard procedures. The cell lines in panel 3D and 1.3D are of the most common cell lines used in the scientific literature.

Panels 4D, 4R, and 4.1D

Panel 4 includes samples on a 96 well plate (2 control wells, 94 test samples) composed of RNA (Panel 4R) or cDNA (Panels 4D/4.1D) isolated from various human cell lines or tissues related to inflammatory conditions. Total RNA from control normal tissues such as colon and lung (Stratagene, La Jolla, Calif.) and thymus and kidney (Clontech) was employed. Total RNA from liver tissue from cirrhosis patients and kidney from lupus patients was obtained from BioChain (Biochain Institute, Inc., Hayward, Calif.). Intestinal tissue for RNA preparation from patients diagnosed as having Crohn's disease and ulcerative colitis was obtained from the National Disease Research Interchange (NDRI) (Philadelphia, Pa.).

Astrocytes, lung fibroblasts, dermal fibroblasts, coronary artery smooth muscle cells, small airway epithelium, bronchial epithelium, microvascular dermal endothelial cells, microvascular lung endothelial cells, human pulmonary aortic endothelial cells, human umbilical vein endothelial cells were all purchased from Clonetics (Walkersville, Md.) and grown in the media supplied for these cell types by Clonetics. These primary cell types were activated with various cytokines or combinations of cytokines for 6 and/or 12-14 hours, as indicated. The following cytokines were used; IL-1 beta at approximately 1-5 ng/ml, TNF alpha at approximately 5-10 ng/ml, IFN gamma at approximately 20-50 ng/ml, IL-4 at approximately 5-10 ng/ml, IL-9 at approximately 5-10 ng/ml, IL-13 at approximately 5-10 ng/ml. Endothelial cells were sometimes starved for various times by culture in the basal media from Clonetics with 0.1% serum.

Mononuclear cells were prepared from blood of employees at CuraGen Corporation, using Ficoll. LAK cells were prepared from these cells by culture in DMEM 5% FCS (Hyclone), 100 μM non essential amino acids (Gibco/Life Technologies, Rockville, Md.), 1 mM sodium pyruvate (Gibco), mercaptoethanol 5.5×10⁻⁵M (Gibco), and 10 mM Hepes (Gibco) and Interleukin 2 for 4-6 days. Cells were then either activated with 10-20 ng/ml PMA and 1-2 μg/ml ionomycin, IL-12 at 5-10 ng/ml, IFN gamma at 20-50 ng/ml and IL-18 at 5-10 ng/ml for 6 hours. In some cases, mononuclear cells were cultured for 4-5 days in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco) with PHA (phytohemagglutinin) or PWM (pokeweed mitogen) at approximately 5 μg/ml. Samples were taken at 24, 48 and 72 hours for RNA preparation. MLR (mixed lymphocyte reaction) samples were obtained by taking blood from two donors, isolating the mononuclear cells using Ficoll and mixing the isolated mononuclear cells 1:1 at a final concentration of approximately 2×10⁶ cells/ml in DMEM 5% FCS (Hyclone), 100 μM non essential amino acids (Gibco), 1 mM sodium pyruvate (Gibco), mercaptoethanol (5.5×10⁻⁵M) (Gibco), and 10 mM Hepes (Gibco). The MLR was cultured and samples taken at various time points ranging from 1-7 days for RNA preparation.

Monocytes were isolated from mononuclear cells using CD14 Miltenyi Beads, +ve VS selection columns and a Vario Magnet according to the manufacturer's instructions. Monocytes were differentiated into dendritic cells by culture in DMEM 5% fetal calf serum (FCS) (Hyclone, Logan, Utah), 100 μM non essential amino acids (Gibco), 1 mM sodium pyruvate (Gibco), mercaptoethanol 5.5×10⁻⁵M (Gibco), and 10 mM Hepes (Gibco), 50 ng/ml GMCSF and 5 ng/ml IL-4 for 5-7 days. Macrophages were prepared by culture of monocytes for 5-7 days in DMEM 5% FCS (Hyclone), 100 μM non essential amino acids (Gibco), 1 mM sodium pyruvate (Gibco), mercaptoethanol 5.5×10⁻⁵M (Gibco), 10 mM Hepes (Gibco) and 10% AB Human Serum or MCSF at approximately 50 ng/ml. Monocytes, macrophages and dendritic cells were stimulated for 6 and 12-14 hours with lipopolysaccharide (LPS) at 100 ng/ml. Dendritic cells were also stimulated with anti-CD40 monoclonal antibody (Pharmingen) at10 μg/ml for 6 and 12-14 hours.

CD4 lymphocytes, CD8 lymphocytes and NK cells were also isolated from mononuclear cells using CD4, CD8 and CD56 Miltenyi beads, positive VS selection columns and a Vario Magnet according to the manufacturer's instructions. CD45RA and CD45RO CD4 lymphocytes were isolated by depleting mononuclear cells of CD8, CD56, CD14 and CD19 cells using CD8, CD56, CD14 and CD19 Miltenyi beads and positive selection. CD45RO beads were then used to isolate the CD45RO CD4 lymphocytes with the remaining cells being CD45RA CD4 lymphocytes. CD45RA CD4, CD45RO CD4 and CD8 lymphocytes were placed in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco) and plated at 10⁶ cells/ml onto Falcon 6 well tissue culture plates that had been coated overnight with 0.5 μg/ml anti-CD28 (Pharmingen) and 3 μg/ml anti-CD3 (OKT3, ATCC) in PBS. After 6 and 24 hours, the cells were harvested for RNA preparation. To prepare chronically activated CD8 lymphocytes, we activated the isolated CD8 lymphocytes for 4 days on anti-CD28 and anti-CD3 coated plates and then harvested the cells and expanded them in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco) and IL-2. The expanded CD8 cells were then activated again with plate bound anti-CD3 and anti-CD28 for 4 days and expanded as before. RNA was isolated 6 and 24 hours after the second activation and after 4 days of the second expansion culture. The isolated NK cells were cultured in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco) and IL-2 for 4-6 days before RNA was prepared.

To obtain B cells, tonsils were procured from NDRI. The tonsil was cut up with sterile dissecting scissors and then passed through a sieve. Tonsil cells were then spun down and resupended at 10⁶ cells/ml in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco). To activate the cells, we used PWM at 5 μg/ml or anti-CD40 (Pharmingen) at approximately 10 μg/ml and IL-4 at 5-10 ng/ml. Cells were harvested for RNA preparation at 24,48 and 72 hours.

To prepare the primary and secondary Th1/Th2 and Tr1 cells, six-well Falcon plates were coated overnight with 10 μg/ml anti-CD28 (Pharmingen) and 2 μg/ml OKT3 (ATCC), and then washed twice with PBS. Umbilical cord blood CD4 lymphocytes (Poietic Systems, German Town, Md.) were cultured at 10⁵-10⁶ cells/ml in DMEM 5% FCS (Hyclone), 100 μM non essential amino acids (Gibco), 1 mM sodium pyruvate (Gibco), mercaptoethanol 5.5×10⁻⁵M (Gibco), 10 mM Hepes (Gibco) and IL-2 (4 ng/ml). IL-12 (5 ng/ml) and anti-IL4 (1 μg/ml) were used to direct to Th1, while IL-4 (5 ng/ml) and anti-IFN gamma (1 μg/ml) were used to direct to Th2 and IL-10 at 5 ng/ml was used to direct to Tr1. After 4-5 days, the activated Th1, Th2 and Tr1 lymphocytes were washed once in DMEM and expanded for 4-7 days in DMEM 5% FCS (Hyclone), 100 μM non essential amino acids (Gibco), 1 mM sodium pyruvate (Gibco), mercaptoethanol 5.5×10⁻⁵M (Gibco), 10 mM Hepes (Gibco) and IL-2 (1 ng/ml). Following this, the activated Th1, Th2 and Tr1 lymphocytes were re-stimulated for 5 days with anti-CD28/OKT3 and cytokines as described above, but with the addition of anti-CD95L (1 g/ml) to prevent apoptosis. After 4-5 days, the Th1, Th2 and Tr1 lymphocytes were washed and then expanded again with IL-2 for 4-7 days. Activated Th1 and Th2 lymphocytes were maintained in this way for a maximum of three cycles. RNA was prepared from primary and secondary Th1, Th2 and Tr1 after 6 and 24 hours following the second and third activations with plate bound anti-CD3 and anti-CD28 mAbs and 4 days into the second and third expansion cultures in Interleukin 2.

The following leukocyte cells lines were obtained from the ATCC: Ramos, EOL-1, KU-812. EOL cells were further differentiated by culture in 0.1 mM dbcAMP at 5×10⁵ cells/ml for 8 days, changing the media every 3 days and adjusting the cell concentration to 5×10⁵ cells/ml. For the culture of these cells, we used DMEM or RPMI (as recommended by the ATCC), with the addition of 5% FCS (Hyclone), 100 μM non essential amino acids (Gibco),1 mM sodium pyruvate (Gibco), mercaptoethanol 5.5×10⁻⁵M (Gibco), 10 mM Hepes (Gibco). RNA was either prepared from resting cells or cells activated with PMA at 10 ng/ml and ionomycin at 1 μg/ml for 6 and 14 hours. Keratinocyte line CCD106 and an airway epithelial tumor line NCI-H292 were also obtained from the ATCC. Both were cultured in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco). CCD1106 cells were activated for 6 and 14 hours with approximately 5 ng/ml TNF alpha and 1 ng/ml IL-1 beta, while NCI-H292 cells were activated for 6 and 14 hours with the following cytokines: 5 ng/ml IL-4, 5 ng/ml IL-9, 5 ng/ml IL-13 and 25 ng/ml IFN gamma.

For these cell lines and blood cells, RNA was prepared by lysing approximately 10⁷ cells/ml using Trizol (Gibco BRL). Briefly, 1/10 volume of bromochloropropane (Molecular Research Corporation) was added to the RNA sample, vortexed and after 10 minutes at room temperature, the tubes were spun at 14,000 rpm in a Sorvall SS34 rotor. The aqueous phase was removed and placed in a 15 ml Falcon Tube. An equal volume of isopropanol was added and left at −20° C. overnight. The precipitated RNA was spun down at 9,000 rpm for 15 min in a Sorvall SS34 rotor and washed in 70% ethanol. The pellet was redissolved in 300 μl of RNAse-free water and 35 μl buffer (Promega) 5 μl DTT, 7 μl RNAsin and 8 μl DNAse were added. The tube was incubated at 37° C. for 30 minutes to remove contaminating genomic DNA, extracted once with phenol chloroform and re-precipitated with 1/10 volume of 3M sodium acetate and 2 volumes of 100% ethanol. The RNA was spun down and placed in RNAse free water. RNA was stored at −80° C.

AI_comprehensive panel_v1.0

The plates for AI_comprehensive panel_v1.0 include two control wells and 89 test samples comprised of cDNA isolated from surgical and postmortem human tissues obtained from the Backus Hospital and Clinomics (Frederick, Md.). Total RNA was extracted from tissue samples from the Backus Hospital in the Facility at CuraGen. Total RNA from other tissues was obtained from Clinomics.

Joint tissues including synovial fluid, synovium, bone and cartilage were obtained from patients undergoing total knee or hip replacement surgery at the Backus Hospital. Tissue samples were immediately snap frozen in liquid nitrogen to ensure that isolated RNA was of optimal quality and not degraded. Additional samples of osteoarthritis and rheumatoid arthritis joint tissues were obtained from Clinomics. Normal control tissues were supplied by Clinomics and were obtained during autopsy of trauma victims.

Surgical specimens of psoriatic tissues and adjacent matched tissues were provided as total RNA by Clinomics. Two male and two female patients were selected between the ages of 25 and 47. None of the patients were taking prescription drugs at the time samples were isolated.

Surgical specimens of diseased colon from patients with ulcerative colitis and Crohns disease and adjacent matched tissues were obtained from Clinomics. Bowel tissue from three female and three male Crohn's patients between the ages of 41-69 were used. Two patients were not on prescription medication while the others were taking dexamethasone, phenobarbital, or tylenol. Ulcerative colitis tissue was from three male and four female patients. Four of the patients were taking lebvid and two were on phenobarbital.

Total RNA from post mortem lung tissue from trauma victims with no disease or with emphysema, asthma or COPD was purchased from Clinomics. Emphysema patients ranged in age from 40-70 and all were smokers, this age range was chosen to focus on patients with cigarette-linked emphysema and to avoid those patients with alpha-1 anti-trypsin deficiencies. Asthma patients ranged in age from 36-75, and excluded smokers to prevent those patients that could also have COPD. COPD patients ranged in age from 35-80 and included both smokers and non-smokers. Most patients were taking corticosteroids, and bronchodilators.

In the labels employed to identify tissues in the AI_comprehensive panel_v1.0 panel, the following abbreviations are used:

AI=Autoimmunity

Syn=Synovial

Normal=No apparent disease

Rep22/Rep20=individual patients

RA=Rheumatoid arthritis

Backus=From Backus Hospital

OA=Osteoarthritis

(SS) (BA) (MF)=Individual patients

Adj=Adjacent tissue

Match control=adjacent tissues

-M=Male

-F=Female

COPD=Chronic obstructive pulmonary disease

Panels 5D and 5I

The plates for Panel 5D and 5I include two control wells and a variety of cDNAs isolated from human tissues and cell lines with an emphasis on metabolic diseases. Metabolic tissues were obtained from patients enrolled in the Gestational Diabetes study. Cells were obtained during different stages in the differentiation of adipocytes from human mesenchymal stem cells. Human pancreatic islets were also obtained.

In the Gestational Diabetes study subjects are young (18-40 years), otherwise healthy women with and without gestational diabetes undergoing routine (elective) Caesarean section. After delivery of the infant, when the surgical incisions were being repaired/closed, the obstetrician removed a small sample (<1 cc) of the exposed metabolic tissues during the closure of each surgical level. The biopsy material was rinsed in sterile saline, blotted and fast frozen within 5 minutes from the time of removal. The tissue was then flash frozen in liquid nitrogen and stored, individually, in sterile screw-top tubes and kept on dry ice for shipment to or to be picked up by CuraGen. The metabolic tissues of interest include uterine wall (smooth muscle), visceral adipose, skeletal muscle (rectus) and subcutaneous adipose. Patient descriptions are as follows:

| | |
|---|---|
| Patient 2 | Diabetic Hispanic, overweight, not on insulin |
| Patient 7-9 | Nondiabetic Caucasian and obese (BMI > 30) |
| Patient 10 | Diabetic Hispanic, overweight, on insulin |
| Patient 11 | Nondiabetic African American and overweight |
| Patient 12 | Diabetic Hispanic on insulin |

Adipocyte differentiation was induced in donor progenitor cells obtained from Osirus (a division of Clonetics/BioWhittaker) in triplicate, except for Donor 3U which had only two replicates. Scientists at Clonetics isolated, grew and differentiated human mesenchymal stem cells (HuMSCs) for CuraGen based on the published protocol found in Mark F. Pittenger, et al., Multilineage Potential of Adult Human Mesenchymal Stem Cells Science Apr. 2, 1999: 143-147. Clonetics provided Trizol lysates or frozen pellets suitable for mRNA isolation and ds cDNA production. A general description of each donor is as follows:

Donor 2 and 3 U: Mesenchymal Stem cells, Undifferentiated Adipose

Donor 2 and 3 AM: Adipose, AdiposeMidway Differentiated

Donor 2 and 3 AD: Adipose, Adipose Differentiated

Human cell lines were generally obtained from ATCC (American Type Culture Collection), NCI or the German tumor cell bank and fall into the following tissue groups: kidney proximal convoluted tubule, uterine smooth muscle cells, small intestine, liver HepG2 cancer cells, heart primary stromal cells, and adrenal cortical adenoma cells. These cells are all cultured under standard recommended conditions and RNA extracted using the standard procedures. All samples were processed at CuraGen to produce single stranded cDNA.

Panel 5I contains all samples previously described with the addition of pancreatic islets from a 58 year old female patient obtained from the Diabetes Research Institute at the University of Miami School of Medicine. Islet tissue was processed to total RNA at an outside source and delivered to CuraGen for addition to panel 51.

In the labels employed to identify tissues in the 5D and 5I panels, the following abbreviations are used:

GO Adipose=Greater Omentum Adipose

SK=Skeletal Muscle

UT=Uterus

PL=Placenta

AD=Adipose Differentiated

AM=Adipose Midway Differentiated

U=Undifferentiated Stem Cells

Panel CNSD.01

The plates for Panel CNSD.01 include two control wells and 94 test samples comprised of cDNA isolated from postmortem human brain tissue obtained from the Harvard Brain Tissue Resource Center. Brains are removed from calvaria of donors between 4 and 24 hours after death, sectioned by neuroanatomists, and frozen at −80° C. in liquid nitrogen vapor. All brains are sectioned and examined by neuropathologists to confirm diagnoses with clear associated neuropathology.

Disease diagnoses are taken from patient records. The panel contains two brains from each of the following diagnoses: Alzheimer's disease, Parkinson's disease, Huntington's disease, Progressive Supernuclear Palsy, Depression, and “Normal controls”. Within each of these brains, the following regions are represented: cingulate gyrus, temporal pole, globus palladus, substantia nigra, Brodman Area 4 (primary motor strip), Brodman Area 7 (parietal cortex), Brodman Area 9 (prefrontal cortex), and Brodman area 17 (occipital cortex). Not all brain regions are represented in all cases; e.g., Huntington's disease is characterized in part by neurodegeneration in the globus palladus, thus this region is impossible to obtain from confirmed Huntington's cases. Likewise Parkinson's disease is characterized by degeneration of the substantia nigra making this region more difficult to obtain. Normal control brains were examined for neuropathology and found to be free of any pathology consistent with neurodegeneration.

In the labels employed to identify tissues in the CNS panel, the following abbreviations are used:

PSP=Progressive supranuclear palsy

Sub Nigra=Substantia nigra

Glob Palladus=Globus palladus

Temp Pole=Temporal pole

Cing Gyr=Cingulate gyrus

BA 4=Brodman Area 4

Panel CNS_Neurodegeneration_V1.0

The plates for Panel CNS_Neurodegeneration_V1.0 include two control wells and 47 test samples comprised of cDNA isolated from postmortem human brain tissue obtained from the Harvard Brain Tissue Resource Center (McLean Hospital) and the Human Brain and Spinal Fluid Resource Center (VA Greater Los Angeles Healthcare System). Brains are removed from calvaria of donors between 4 and 24 hours after death, sectioned by neuroanatomists, and frozen at −80° C. in liquid nitrogen vapor. All brains are sectioned and examined by neuropathologists to confirm diagnoses with clear associated neuropathology.

Disease diagnoses are taken from patient records. The panel contains six brains from Alzheimer's disease (AD) patients, and eight brains from “Normal controls” who showed no evidence of dementia prior to death. The eight normal control brains are divided into two categories: Controls with no dementia and no Alzheimer's like pathology (Controls) and controls with no dementia but evidence of severe Alzheimer's like pathology, (specifically senile plaque load rated as level 3 on a scale of 0-3; 0=no evidence of plaques, 3=severe AD senile plaque load). Within each of these brains, the following regions are represented: hippocampus, temporal cortex (Brodman Area 21), parietal cortex (Brodman area 7), and occipital cortex (Brodman area 17). These regions were chosen to encompass all levels of neurodegeneration in AD. The hippocampus is a region of early and severe neuronal loss in AD; the temporal cortex is known to show neurodegeneration in AD after the hippocampus; the parietal cortex shows moderate neuronal death in the late stages of the disease; the occipital cortex is spared in AD and therefore acts as a “control” region within AD patients. Not all brain regions are represented in all cases.

In the labels employed to identify tissues in the CNS_Neurodegeneration_V1.0 panel, the following abbreviations are used:

AD=Alzheimer's disease brain; patient was demented and showed AD-like pathology upon autopsy

Control=Control brains; patient not demented, showing no neuropathology

Control (Path)=Control brains; patient not demented but showing sever AD-like pathology

SupTemporal Ctx=Superior Temporal Cortex

Inf Temporal Ctx=Inferior Temporal Cortex

NOV9a and NOV9b

Expression of gene NOV9a and variant NOV9b was assessed using the primer-probe sets Ag2930,Ag4297 and Ag573, described in Tables AA, AB and AC. Results of the RTQ-PCR runs are shown in Tables AD, AE, AF, AG, and AH. Please note that the probe and primer set Ag4297 do not match the NOV9b variant.

TABLE AA
Probe Name Ag2930

| | | | Start | |
|---|---|---|---|---|
| Primers | Sequences | Length | Position | SEQ ID NO: |
| Forward | 5′-attggattttccaactccatct-3′ | 22 | 1213 | 994 |
| Probe | TET-5′-tcccattgtctatgcatttatgaatga-3′-TAMRA | 27 | 1239 | 995 |
| Reverse | 5′-tgcaataacaaactgcagacaa-3′ | 22 | 1285 | 996 |

TABLE AB
Probe Name Ag4297

| | | | Start | |
|---|---|---|---|---|
| Primers | Sequences | Length | Position | SEQ ID NO: |
| Forward | 5′-tctacaccaccttcatccttgt-3′ | 22 | 1007 | 997 |
| Probe | TET-5′-ctgcctcttatggagaagaaacgagctg-3′-TAMRA | 28 | 1042 | 998 |
| Reverse | 5′-caccactgtcaccatcataatg-3′ | 22 | 1071 | 999 |

TABLE AC
Probe Name Ag573

| | | | Start | |
|---|---|---|---|---|
| Primers | Sequences | Length | Position | SEQ ID NO: |
| Forward | 5′-tgtccagtctaccgctgttgtg-3′ | 22 | 738 | 1000 |
| Probe | TET-5′-agaaatcctcactatgacctgcattgctgtg-3′-TAMRA | 31 | 762 | |
| Reverse | 5′-cacaagtccctggtgccttt-3′ | 20 | 794 | 1002 |

TABLE AD
Panel 1.1

| | Rel. Exp. (%) | Rel. Exp. (%) | | Rel. Exp. (%) | Rel. Exp. (%) |
|---|---|---|---|---|---|
| | Ag573, Run | Ag573, Run | | Ag573, Run | Ag573, Run |
| Tissue Name | 109566077 | 111156622 | Tissue Name | 109566077 | 111156622 |
| Adrenal gland | 0.0 | 0.8 | Renal ca. UO- | 0.1 | 6.0 |
| | | | 31 | | |
| Bladder | 0.0 | 0.0 | Renal ca. RXF | 0.0 | 0.0 |
| | | | 393 | | |
| Brain | 0.0 | 0.6 | Liver | 0.0 | 0.0 |
| (amygdala) | | | | | |
| Brain | 0.1 | 0.1 | Liver (fetal) | 0.0 | 0.0 |
| (cerebellum) | | | | | |
| Brain | 0.0 | 5.3 | Liver ca. | 0.0 | 0.0 |
| (hippocampus) | | | (hepatoblast) | | |
| | | | HepG2 | | |
| Brain (substantia | 10.7 | 13.7 | Lung | 0.0 | 0.0 |
| nigra) | | | | | |
| Brain (thalamus) | 0.1 | 4.9 | Lung (fetal) | 0.0 | 1.5 |
| Cerebral Cortex | 9.3 | 11.2 | Lung ca. (non- | 4.5 | 0.0 |
| | | | s.cell) HOP-62 | | |
| Brain (fetal) | 0.0 | 0.8 | Lung ca. (large | 0.0 | 0.0 |
| | | | cell)NCI-H460 | | |
| Brain (whole) | 1.3 | 6.0 | Lung ca. (non- | 0.0 | 0.0 |
| | | | s.cell) NCI- | | |
| | | | H23 | | |
| glio/astro U-118- | 0.0 | 0.0 | Lung ca. (non- | 2.2 | 0.0 |
| MG | | | s.cl) NCI- | | |
| | | | H522 | | |
| astrocytoma SF- | 0.0 | 1.3 | Lung ca. (non- | 0.0 | 0.0 |
| 539 | | | sm. cell) A549 | | |
| astrocytoma | 0.0 | 0.0 | Lung ca. (s.cell | 0.0 | 0.0 |
| SNB-75 | | | var.) SHP-77 | | |
| astrocytoma | 0.0 | 0.0 | Lung ca. | 0.0 | 0.0 |
| SW1783 | | | (small cell) | | |
| | | | LX-1 | | |
| glioma U251 | 0.0 | 0.0 | Lung ca. | 43.5 | 47.0 |
| | | | (small cell) | | |
| | | | NCI-H69 | | |
| glioma SF-295 | 1.3 | 0.0 | Lung ca. | 0.0 | 0.0 |
| | | | (squam.) SW | | |
| | | | 900 | | |
| glioma SNB-19 | 0.0 | 0.5 | Lung ca. | 100.0 | 98.6 |
| | | | (squam.) NCI- | | |
| | | | H596 | | |
| glio/astro U87- | 0.0 | 1.5 | Lymph node | 0.0 | 0.0 |
| MG | | | | | |
| neuro*; met SK- | 2.6 | 0.1 | Spleen | 0.0 | 0.0 |
| N-AS | | | | | |
| Mammary gland | 0.0 | 3.8 | Thymus | 0.0 | 0.0 |
| Breast ca. BT- | 0.0 | 0.3 | Ovary | 0.0 | 0.6 |
| 549 | | | | | |
| Breast ca. MDA-N | 28.3 | 39.8 | Ovarian ca. | 0.0 | 0.0 |
| | | | IGROV-1 | | |
| Breast ca.* | 0.0 | 0.0 | Ovarian ca. | 0.2 | 1.8 |
| (pl.ef) T47D | | | OVCAR-3 | | |
| Breast ca.* | 0.3 | 0.0 | Ovarian ca. | 0.0 | 0.0 |
| (pl.ef) MCF-7 | | | OVCAR-4 | | |
| Breast ca.* | 0.0 | 0.0 | Ovarian ca. | 0.0 | 0.1 |
| (pl.ef) MDA- | | | OVCAR-5 | | |
| MB-231 | | | | | |
| Small intestine | 0.0 | 0.0 | Ovarian ca. | 2.5 | 16.3 |
| | | | OVCAR-8 | | |
| Colorectal | 0.0 | 0.0 | Ovarian ca.* | 0.8 | 4.6 |
| | | | (ascites) SK- | | |
| | | | OV-3 | | |
| Colon ca. HT29 | 0.0 | 0.0 | Pancreas | 2.5 | 0.0 |
| Colon ca. CaCo-2 | 0.0 | 0.0 | Pancreatic ca. | 0.0 | 0.0 |
| | | | CAPAN 2 | | |
| Colon ca. HCT- | 0.0 | 0.0 | Pituitary gland | 1.5 | 2.7 |
| 15 | | | | | |
| Colon ca. HCT- | 0.0 | 0.0 | Placenta | 0.0 | 0.0 |
| 116 | | | | | |
| Colon ca. HCC- | 0.0 | 0.0 | Prostate | 0.0 | 0.0 |
| 2998 | | | | | |
| Colon ca. | 0.0 | 0.0 | Prostate ca.* | 0.1 | 0.0 |
| SW480 | | | (bone met) PC-3 | | |
| Colon ca.* | 1.9 | 0.0 | Salivary gland | 0.1 | 0.0 |
| SW620 (SW480 | | | | | |
| met) | | | | | |
| Stomach | 0.0 | 0.0 | Trachea | 0.0 | 0.0 |
| Gastric ca. (liver | 0.1 | 0.0 | Spinal cord | 0.0 | 2.4 |
| met) NCI-N87 | | | | | |
| Heart | 3.8 | 19.6 | Testis | 0.0 | 2.0 |
| Skeletal muscle | 0.0 | 0.3 | Thyroid | 0.0 | 2.3 |
| (Fetal) | | | | | |
| Skeletal muscle | 0.0 | 0.0 | Uterus | 0.0 | 0.0 |
| Endothelial cells | 0.0 | 0.0 | Melanoma | 0.0 | 0.5 |
| | | | M14 | | |
| Heart (Fetal) | 11.0 | 31.0 | Melanoma | 0.0 | 0.0 |
| | | | LOX IMVI | | |
| Kidney | 1.3 | 12.2 | Melanoma | 0.0 | 0.0 |
| | | | UACC-62 | | |
| Kidney (fetal) | 0.0 | 3.9 | Melanoma SK- | 0.0 | 0.4 |
| | | | MEL-28 | | |
| Renal ca. 786-0 | 0.0 | 5.4 | Melanoma* | 0.0 | 0.0 |
| | | | (met) SK- | | |
| | | | MEL-5 | | |
| Renal ca. A498 | 10.5 | 16.4 | Melanoma | 0.0 | 0.2 |
| | | | Hs688(A).T | | |
| Renal ca. ACHN | 85.3 | 100.0 | Melanoma* | 0.0 | 0.0 |
| | | | (met) | | |
| | | | Hs688(B).T | | |
| Renal ca. TK-10 | 60.7 | 59.0 | | | |

TABLE AE
Panel 1.3D

| | Rel. Exp. (%) | Rel. Exp. (%) | | Rel. Exp. (%) | Rel. Exp. (%) |
|---|---|---|---|---|---|
| | Ag2930, Run | Ag2930, Run | | Ag2930, Run | Ag2930, Run |
| Tissue Name | 158090377 | 165701939 | Tissue Name | 158090377 | 165701939 |
| Liver | 0.0 | 0.0 | Kidney (fetal) | 10.1 | 0.0 |
| adenocarcinoma | | | | | |
| Pancreas | 0.0 | 0.0 | Renal ca. 786-0 | 2.3 | 7.8 |
| Pancreatic ca. | 0.0 | 0.0 | Renal ca. | 61.6 | 44.8 |
| CAPAN 2 | | | A498 | | |
| Adrenal gland | 0.0 | 0.0 | Renal ca. RXF | 0.0 | 0.0 |
| | | | 393 | | |
| Thyroid | 1.6 | 0.0 | Renal ca. | 16.8 | 100.0 |
| | | | ACHN | | |
| Salivary gland | 0.0 | 0.0 | Renal ca. UO- | 10.1 | 15.0 |
| | | | 31 | | |
| Pituitary gland | 3.1 | 0.0 | Renal ca. TK- | 22.5 | 52.5 |
| | | | 10 | | |
| Brain (fetal) | 9.7 | 3.6 | Liver | 0.0 | 0.0 |
| Brain (whole) | 11.0 | 24.3 | Liver (fetal) | 1.3 | 0.0 |
| Brain (amygdala) | 23.2 | 9.3 | Liver ca. | 0.0 | 0.0 |
| | | | (hepatoblast) | | |
| | | | HepG2 | | |
| Brain (cerebellum) | 0.0 | 0.0 | Lung | 0.0 | 0.0 |
| Brain | 100.0 | 3.4 | Lung (fetal) | 0.0 | 15.0 |
| (hippocampus) | | | | | |
| Brain (substantia | 2.7 | 19.5 | Lung ca. | 0.0 | 0.0 |
| nigra) | | | (small cell) | | |
| | | | LX-1 | | |
| Brain (thalamus) | 23.3 | 6.8 | Lung ca. | 39.2 | 16.0 |
| | | | (small cell) | | |
| | | | NCI-H69 | | |
| Cerebral Cortex | 33.4 | 0.0 | Lung ca. | 0.0 | 0.0 |
| | | | (s.cell var.) | | |
| | | | SHP-77 | | |
| Spinal cord | 6.4 | 7.0 | Lung ca. (large | 0.0 | 0.0 |
| | | | cell)NCI-H460 | | |
| glio/astro U87-MG | 2.6 | 4.9 | Lung ca. (non- | 0.0 | 0.0 |
| | | | sm. cell) A549 | | |
| glio/astro U-118- | 0.0 | 0.0 | Lung ca. (non- | 0.0 | 0.0 |
| MG | | | s.cell) NCI- | | |
| | | | H23 | | |
| astrocytoma | 0.0 | 0.0 | Lung ca. (non- | 0.0 | 0.0 |
| SW1783 | | | s.cell) HOP-62 | | |
| neuro*; met SK-N- | 4.2 | 0.0 | Lung ca. (non- | 0.0 | 0.0 |
| AS | | | s.cl) NCI- | | |
| | | | H522 | | |
| astrocytoma SF- | 5.0 | 0.0 | Lung ca. | 0.0 | 0.0 |
| 539 | | | (squam.) SW | | |
| | | | 900 | | |
| astrocytoma SNB- | 0.0 | 0.0 | Lung ca. | 8.4 | 6.7 |
| 75 | | | (squam.) NCI- | | |
| | | | H596 | | |
| glioma SNB-19 | 2.1 | 0.0 | Mammary | 18.3 | 0.0 |
| | | | gland | | |
| glioma U251 | 0.0 | 0.0 | Breast ca.* | 1.4 | 4.6 |
| | | | (pl.ef) MCF-7 | | |
| glioma SF-295 | 0.0 | 0.0 | Breast ca.* | 0.0 | 0.0 |
| | | | (pl.ef) MDA- | | |
| | | | MB-231 | | |
| Heart (fetal) | 18.4 | 0.0 | Breast ca.* | 0.0 | 0.0 |
| | | | (pl.ef) T47D | | |
| Heart | 2.4 | 4.1 | Breast ca. BT- | 10.7 | 5.8 |
| | | | 549 | | |
| Skeletal muscle | 1.5 | 0.0 | Breast ca. | 35.1 | 4.7 |
| (fetal) | | | MDA-N | | |
| Skeletal muscle | 0.0 | 0.0 | Ovary | 0.0 | 0.0 |
| Bone marrow | 0.0 | 0.0 | Ovarian ca. | 0.0 | 0.0 |
| | | | OVCAR-3 | | |
| Thymus | 0.0 | 0.0 | Ovarian ca. | 0.0 | 0.0 |
| | | | OVCAR-4 | | |
| Spleen | 1.8 | 0.0 | Ovarian ca. | 0.0 | 0.0 |
| | | | OVCAR-5 | | |
| Lymph node | 0.0 | 11.6 | Ovarian ca. | 12.9 | 4.9 |
| | | | OVCAR-8 | | |
| Colorectal | 0.0 | 0.0 | Ovarian ca. | 0.0 | 0.0 |
| | | | IGROV-1 | | |
| Stomach | 1.3 | 3.3 | Ovarian ca.* | 2.9 | 6.5 |
| | | | (ascites) SK- | | |
| | | | OV-3 | | |
| Small intestine | 1.7 | 0.0 | Uterus | 0.0 | 0.0 |
| Colon ca. SW480 | 0.0 | 0.0 | Placenta | 0.0 | 0.0 |
| Colon ca.* | 0.0 | 0.0 | Prostate | 0.0 | 0.0 |
| SW620 (SW480 | | | | | |
| met) | | | | | |
| Colon ca. HT29 | 0.0 | 0.0 | Prostate ca.* | 0.0 | 0.0 |
| | | | (bone met) PC-3 | | |
| Colon ca. HCT- | 0.0 | 0.0 | Testis | 2.4 | 0.0 |
| 116 | | | | | |
| Colon ca. CaCo-2 | 0.0 | 0.0 | Melanoma | 0.0 | 0.0 |
| | | | Hs688(A).T | | |
| Colon ca. | 0.0 | 0.0 | Melanoma* | 0.0 | 0.0 |
| tissue(ODO3866) | | | (met) | | |
| | | | Hs688(B).T | | |
| Colon ca. HCC- | 0.0 | 0.0 | Melanoma | 0.0 | 0.0 |
| 2998 | | | UACC-62 | | |
| Gastric ca.* (liver | 0.0 | 0.0 | Melanoma | 0.0 | 0.0 |
| met) NCI-N87 | | | M14 | | |
| Bladder | 0.0 | 0.0 | Melanoma | 0.0 | 0.0 |
| | | | LOX IMVI | | |
| Trachea | 0.0 | 0.0 | Melanoma* | 0.0 | 0.0 |
| | | | (met) SK- | | |
| | | | MEL-5 | | |
| Kidney | 0.0 | 8.5 | Adipose | 1.3 | 0.0 |

TABLE AF
Panel 2D

| | Rel. Exp. (%) | | Rel. Exp. (%) |
|---|---|---|---|
| | Ag2930, | | Ag2930, |
| Tissue Name | Run 158090382 | Tissue Name | Run 158090382 |
| Normal Colon | 1.2 | Kidney Margin | 0.6 |
| | | 8120608 | |
| CC Well to Mod Diff | 0.4 | Kidney Cancer | 0.0 |
| (ODO3866) | | 8120613 | |
| CC Margin (ODO3866) | 0.3 | Kidney Margin | 0.0 |
| | | 8120614 | |
| CC Gr.2 rectosigmoid | 0.0 | Kidney Cancer | 3.2 |
| (ODO3868) | | 9010320 | |
| CC Margin (ODO3868) | 0.0 | Kidney Margin | 1.7 |
| | | 9010321 | |
| CC Mod Diff (ODO3920) | 0.0 | Normal Uterus | 0.0 |
| CC Margin (ODO3920) | 0.0 | Uterus Cancer 064011 | 0.0 |
| CC Gr.2 ascend colon | 0.0 | Normal Thyroid | 0.5 |
| (ODO3921) | | | |
| CC Margin (ODO3921) | 2.2 | Thyroid Cancer | 0.3 |
| | | 064010 | |
| CC from Partial | 0.0 | Thyroid Cancer | 0.2 |
| Hepatectomy (ODO4309) | | A302152 | |
| Mets | | | |
| Liver Margin (ODO4309) | 0.0 | Thyroid Margin | 0.0 |
| | | A302153 | |
| Colon mets to lung | 0.0 | Normal Breast | 0.0 |
| (OD04451-01) | | | |
| Lung Margin (OD04451- | 0.0 | Breast Cancer | 0.0 |
| 02) | | (OD04566) | |
| Normal Prostate 6546-1 | 0.0 | Breast Cancer | 0.4 |
| | | (OD04590-01) | |
| Prostate Cancer | 0.4 | Breast Cancer Mets | 0.0 |
| (OD04410) | | (OD04590-03) | |
| Prostate Margin | 0.0 | Breast Cancer | 0.0 |
| (OD04410) | | Metastasis | |
| | | (OD04655-05) | |
| Prostate Cancer | 1.0 | Breast Cancer 064006 | 0.0 |
| (OD04720-01) | | | |
| Prostate Margin | 0.0 | Breast Cancer 1024 | 0.2 |
| (OD04720-02) | | | |
| Normal Lung 061010 | 0.2 | Breast Cancer | 3.5 |
| | | 9100266 | |
| Lung Met to Muscle | 0.5 | Breast Margin | 0.3 |
| (ODO4286) | | 9100265 | |
| Muscle Margin | 0.0 | Breast Cancer | 1.0 |
| (ODO4286) | | A209073 | |
| Lung Malignant Cancer | 0.0 | Breast Margin | 0.0 |
| (OD03126) | | A2090734 | |
| Lung Margin (OD03126) | 0.4 | Normal Liver | 0.0 |
| Lung Cancer (OD04404) | 1.9 | Liver Cancer 064003 | 0.2 |
| Lung Margin (OD04404) | 0.0 | Liver Cancer 1025 | 0.0 |
| Lung Cancer (OD04565) | 0.0 | Liver Cancer 1026 | 0.4 |
| Lung Margin (OD04565) | 0.0 | Liver Cancer 6004-T | 0.0 |
| Lung Cancer (OD04237- | 0.0 | Liver Tissue 6004-N | 0.0 |
| 01) | | | |
| Lung Margin (OD04237- | 0.0 | Liver Cancer 6005-T | 0.0 |
| 02) | | | |
| Ocular Mel Met to Liver | 0.0 | Liver Tissue 6005-N | 0.0 |
| (ODO4310) | | | |
| Liver Margin (ODO4310) | 0.0 | Normal Bladder | 0.0 |
| Melanoma Mets to Lung | 0.2 | Bladder Cancer 1023 | 0.0 |
| (OD04321) | | | |
| Lung Margin (OD04321) | 0.0 | Bladder Cancer | 5.0 |
| | | A302173 | |
| Normal Kidney | 1.6 | Bladder Cancer | 0.0 |
| | | (OD04718-01) | |
| Kidney Ca, Nuclear grade | 0.0 | Bladder Normal | 0.0 |
| 2 (OD04338) | | Adjacent (OD04718- | |
| | | 03) | |
| Kidney Margin | 2.7 | Normal Ovary | 0.9 |
| (OD04338) | | | |
| Kidney Ca Nuclear grade | 45.1 | Ovarian Cancer | 0.1 |
| 1/2 (OD04339) | | 064008 | |
| Kidney Margin | 2.4 | Ovarian Cancer | 0.0 |
| (OD04339) | | (OD04768-07) | |
| Kidney Ca, Clear cell | 100.0 | Ovary Margin | 0.0 |
| type (OD04340) | | (OD04768-08) | |
| Kidney Margin | 3.1 | Normal Stomach | 0.0 |
| (OD04340) | | | |
| Kidney Ca, Nuclear grade | 0.0 | Gastric Cancer | 0.0 |
| 3 (OD04348) | | 9060358 | |
| Kidney Margin | 1.3 | Stomach Margin | 0.0 |
| (OD04348) | | 9060359 | |
| Kidney Cancer | 20.9 | Gastric Cancer | 0.0 |
| (OD04622-01) | | 9060395 | |
| Kidney Margin | 0.3 | Stomach Margin | 0.4 |
| (OD04622-03) | | 9060394 | |
| Kidney Cancer | 33.4 | Gastric Cancer | 0.0 |
| (OD04450-01) | | 9060397 | |
| Kidney Margin | 2.4 | Stomach Margin | 0.0 |
| (OD04450-03) | | 9060396 | |
| Kidney Cancer 8120607 | 0.0 | Gastric Cancer | 0.0 |
| | | 064005 | |

TABLE AG
Panel 3D

| | | | Rel. |
|---|---|---|---|
| | Rel. | | Exp. (%) |
| | Exp. (%) Ag2930, | | Ag2930, Run |
| Tissue Name | Run 162374504 | Tissue Name | 162374504 |
| Daoy- | 1.9 | Ca Ski-Cervical | 0.0 |
| Medulloblastoma | | epidermoid carcinoma | |
| | | (metastasis) | |
| TE671- | 0.0 | ES-2-Ovarian clear | 0.0 |
| Medulloblastoma | | cell carcinoma | |
| D283 Med- | 2.0 | Ramos-Stimulated | 0.0 |
| Medulloblastoma | | with PMA/ionomycin 6 h | |
| PFSK-1-Primitive | 0.0 | Ramos-Stimulated | 0.0 |
| Neuroectodermal | | with PMA/ionomycin 14 h | |
| XF-498-CNS | 0.0 | MEG-01-Chronic | 0.0 |
| | | myelogenous leukemia | |
| | | (megokaryoblast) | |
| SNB-78-Glioma | 0.0 | Raji-Burkitt's | 0.0 |
| | | lymphoma | |
| SF-268- | 0.0 | Daudi-Burkitt's | 0.0 |
| Glioblastoma | | lymphoma | |
| T98G- | 0.0 | U266-B-cell | 0.0 |
| Glioblastoma | | plasmacytoma | |
| SK-N-SH- | 0.0 | CA46-Burkitt's | 0.0 |
| Neuroblastoma | | lymphoma | |
| (metastasis) | | | |
| SF-295- | 0.0 | RL-non-Hodgkin's | 0.0 |
| Glioblastoma | | B-cell lymphoma | |
| Cerebellum | 2.7 | JM1-pre-B-cell | 0.0 |
| | | lymphoma | |
| Cerebellum | 7.3 | Jurkat-T cell | 0.0 |
| | | leukemia | |
| NCI-H292- | 0.0 | TF-1- | 0.0 |
| Mucoepidermoid lung | | Erythroleukemia | |
| carcinoma | | | |
| DMS-114-Small | 0.0 | HUT 78-T-cell | 0.0 |
| cell lung cancer | | lymphoma | |
| DMS-79-Small | 0.0 | U937-Histiocytic | 0.0 |
| cell lung cancer | | lymphoma | |
| NCI-H146-Small | 0.0 | KU-812- | 0.0 |
| cell lung cancer | | Myelogenous leukemia | |
| NCI-H526-Small | 0.0 | 769-P-Clear cell | 39.0 |
| cell lung cancer | | renal carcinoma | |
| NCI-N417-Small | 100.0 | Caki-2-Clear cell | 4.6 |
| cell lung cancer | | renal carcinoma | |
| NCI-H82-Small | 0.0 | SW 839-Clear cell | 16.0 |
| cell lung cancer | | renal carcinoma | |
| NCI-H157- | 0.0 | G401-Wilms' tumor | 0.0 |
| Squamous cell lung | | | |
| cancer (metastasis) | | | |
| NCI-H1155- | 0.0 | Hs766T-Pancreatic | 0.0 |
| Large cell lung cancer | | carcinoma (LN metastasis) | |
| NCI-H1299- | 0.0 | CAPAN-1- | 0.0 |
| Large cell lung cancer | | Pancreatic adenocarcinoma | |
| | | (liver metastasis) | |
| NCI-H727-Lung | 0.0 | SU86.86-Pancreatic | 5.0 |
| carcinoid | | carcinoma (liver metastasis) | |
| NCI-UMC-11- | 0.0 | BxPC-3-Pancreatic | 0.0 |
| Lung carcinoid | | adenocarcinoma | |
| LX-1-Small cell | 0.0 | HPAC-Pancreatic | 0.0 |
| lung cancer | | adenocarcinoma | |
| Colo-205-Colon | 0.0 | MIA PaCa-2- | 0.0 |
| cancer | | Pancreatic carcinoma | |
| KM12-Colon | 0.0 | CFPAC-1- | 0.0 |
| cancer | | Pancreatic ductal | |
| | | adenocarcinoma | |
| KM20L2-Colon | 0.0 | PANC-1-Pancreatic | 0.0 |
| cancer | | epithelioid ductal carcinoma | |
| NCI-H716-Colon | 0.0 | T24-Bladder | 0.0 |
| cancer | | carcinma (transitional cell) | |
| SW-48-Colon | 0.0 | 5637-Bladder | 0.0 |
| adenocarcinoma | | carcinoma | |
| SW1116-Colon | 0.0 | HT-1197-Bladder | 0.0 |
| adenocarcinoma | | carcinoma | |
| LS 174T-Colon | 0.0 | UM-UC-3-Bladder | 0.0 |
| adenocarcinoma | | carcinoma (transitional cell) | |
| SW-948-Colon | 0.0 | A204- | 0.0 |
| adenocarcinoma | | Rhabdomyosarcoma | |
| SW-480-Colon | 0.0 | HT-1080- | 0.0 |
| adenocarcinoma | | Fibrosarcoma | |
| NCI-SNU-5- | 0.0 | MG-63- | 0.0 |
| Gastric carcinoma | | Osteosarcoma | |
| KATO III-Gastric | 0.0 | SK-LMS-1- | 0.0 |
| carcinoma | | Leiomyosarcoma (vulva) | |
| NCI-SNU-16- | 0.0 | SJRH30- | 0.0 |
| Gastric carcinoma | | Rhabdomyosarcoma (met to | |
| | | bone marrow) | |
| NCI-SNU-1- | 0.0 | A431-Epidermoid | 0.0 |
| Gastric carcinoma | | carcinoma | |
| RF-1-Gastric | 0.0 | WM266-4- | 0.0 |
| adenocarcinoma | | Melanoma | |
| RF-48-Gastric | 0.0 | DU 145-Prostate | 0.0 |
| adenocarcinoma | | carcinoma (brain metastasis) | |
| MKN-45-Gastric | 8.7 | MDA-MB-468- | 0.0 |
| carcinoma | | Breast adenocarcinoma | |
| NCI-N87-Gastric | 0.0 | SCC-4-Squamous | 0.0 |
| carcinoma | | cell carcinoma of tongue | |
| OVCAR-5- | 0.0 | SCC-9-Squamous | 0.0 |
| Ovarian carcinoma | | cell carcinoma of tongue | |
| RL95-2-Uterine | 0.0 | SCC-15-Squamous | 0.0 |
| carcinoma | | cell carcinoma of tongue | |
| HelaS3-Cervical | 0.0 | CAL 27-Squamous | 0.0 |
| adenocarcinoma | | cell carcinoma of tongue | |

TABLE AH
Panel 4D

| | Rel. Exp. (%) | | Rel. Exp. (%) |
|---|---|---|---|
| | Ag2930, Run | | Ag2930, Run |
| Tissue Name | 158090383 | Tissue Name | 158090383 |
| Secondary Th1 act | 5.9 | HUVEC IL-1beta | 0.0 |
| Secondary Th2 act | 0.0 | HUVEC IFN gamma | 0.0 |
| Secondary Tr1 act | 0.0 | HUVEC TNF alpha + IFN | 0.0 |
| | | gamma | |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC | 9.9 |
| | | none | |
| Primary Th1 act | 0.0 | Lung Microvascular EC | 0.0 |
| | | TNF alpha + IL-1beta | |
| Primary Th2 act | 0.0 | Microvascular Dermal EC | 15.2 |
| | | none | |
| Primary Tr1 act | 0.0 | Microsvasular Dermal EC | 0.0 |
| | | TNF alpha + IL-1beta | |
| Primary Th1 rest | 0.0 | Bronchial epithelium | 0.0 |
| | | TNF alpha + IL1beta | |
| Primary Th2 rest | 0.0 | Small airway epithelium | 0.0 |
| | | none | |
| Primary Tr1 rest | 0.0 | Small airway epithelium | 0.0 |
| | | TNF alpha + IL-1beta | |
| CD45RA CD4 | 0.0 | Coronery artery SMC rest | 0.0 |
| lymphocyte act | | | |
| CD45RO CD4 | 0.0 | Coronery artery SMC | 0.0 |
| lymphocyte act | | TNF alpha + IL-1beta | |
| CD8 lymphocyte act | 0.0 | Astrocytes rest | 0.0 |
| Secondary CD8 | 0.0 | Astrocytes TNF alpha + | 0.0 |
| lymphocyte rest | | IL-1beta | |
| Secondary CD8 | 0.0 | KU-812 (Basophil) rest | 0.0 |
| lymphocyte act | | | |
| CD4 lymphocyte none | 0.0 | KU-812 (Basophil) | 0.0 |
| | | PMA/ionomycin | |
| 2ry Th1/Th2/Tr1_anti- | 0.0 | CCD1106 (Keratinocytes) | 22.1 |
| CD95 CH11 | | none | |
| LAK cells rest | 0.0 | CCD1106 (Keratinocytes) | 8.2 |
| | | TNF alpha + IL-1beta | |
| LAK cells IL-2 | 0.0 | Liver cirrhosis | 0.0 |
| LAK cells IL-2 + IL-12 | 0.0 | Lupus kidney | 10.7 |
| LAK cells IL-2 + IFN | 0.0 | NCI-H292 none | 0.0 |
| gamma | | | |
| LAK cells IL-2 + IL-18 | 0.0 | NCI-H292 IL-4 | 0.0 |
| LAK cells | 0.0 | NCI-H292 IL-9 | 0.0 |
| PMA/ionomycin | | | |
| NK Cells IL-2 rest | 0.0 | NCI-H292 IL-13 | 0.0 |
| Two Way MLR 3 day | 0.0 | NCI-H292 IFN gamma | 0.0 |
| Two Way MLR 5 day | 0.0 | HPAEC none | 0.0 |
| Two Way MLR 7 day | 0.0 | HPAEC TNF alpha + IL-1 | 0.0 |
| | | beta | |
| PBMC rest | 0.0 | Lung fibroblast none | 0.0 |
| PBMC PWM | 0.0 | Lung fibroblast TNF alpha + | 0.0 |
| | | IL-1beta | |
| PBMC PHA-L | 0.0 | Lung fibroblast IL-4 | 0.0 |
| Ramos (B cell) none | 0.0 | Lung fibroblast IL-9 | 0.0 |
| Ramos (B cell) | 0.0 | Lung fibroblast IL-13 | 0.0 |
| ionomycin | | | |
| B lymphocytes PWM | 0.0 | Lung fibroblast IFN | 0.0 |
| | | gamma | |
| B lymphocytes CD40L | 0.0 | Dermal fibroblast | 0.0 |
| and IL-4 | | CCD1070 rest | |
| EOL-1 dbcAMP | 0.0 | Dermal fibroblast | 0.0 |
| | | CCD1070 TNF alpha | |
| EOL-1 dbcAMP | 0.0 | Dermal fibroblast | 0.0 |
| PMA/ionomycin | | CCD1070 IL-1beta | |
| Dendritic cells none | 0.0 | Dermal fibroblast IFN | 0.0 |
| | | gamma | |
| Dendritic cells LPS | 7.9 | Dermal fibroblast IL-4 | 0.0 |
| Dendritic cells anti- | 0.0 | IBD Colitis 2 | 6.3 |
| CD40 | | | |
| Monocytes rest | 0.0 | IBD Crohn's | 0.0 |
| Monocytes LPS | 0.0 | Colon | 0.0 |
| Macrophages rest | 0.0 | Lung | 0.0 |
| Macrophages LPS | 0.0 | Thymus | 100.0 |
| HUVEC none | 0.0 | Kidney | 0.0 |
| HUVEC starved | 0.0 | | |

CNS_neurodegeneration_v1.0 Summary: Ag2930 Expression of the NOV9A gene is low/undetectable in all samples on this panel. (CTs>35). (Data not shown.)

General_screening panel_v1.5 Summary: Ag4297 Expression of the NOV9A gene is low/undetectable in all samples on this panel. (CTs>35). (Data not shown.)

Panel 1.1 Summary: Ag573 Two experiments with the same probe and primer set produce results that are in excellent agreement, with highest expression of the NOV9A gene, a putative neuropeptide Y receptor, in lung cancer and renal cancer cell lines (CTs=23-26). Significant expression is also seen in a cluster of breast cancer cell lines. Thus, expression of this gene could be used to differentiate between these samples and other samples on this panel and as a marker to detect the presence of these cancers. Neuropeptide Y, which controls vasoconstriction and feeding behavior, is expressed in breast cancer (see ref. below). Furthermore, peptide receptors in human tumors represent clinically relevant targets for both cancer diagnosis and treatment. Therefore, therapeutic modulation of the expression or function of this gene may be effective in the treatment of breast, lung and renal cancers.

This molecule, which encodes a neuropeptide Y receptor homolog, is also expressed in the brain. Neuropeptide Y and its receptors have been implicated in feeding behavior, learning and memory, and seizure. This gene would therefore be an excellent small molecule target for the treatment of epilepsy or any seizure disorder.

Among tissues with metabolic function, this gene has low-to-moderate levels of expression in adrenal, heart, fetal skeletal muscle and pancreas. This gene product is highly expressed in fetal and adult heart. Since neuropeptide Y and its receptor are associated with appetite regulation, this gene product may be a small molecule target for the treatment of metabolic and endocrine disease, including obesity and Types 1 and 2 diabetes. In addition, the expression in heart and the suggested role of neuropeptide Y in vasoconstriction, cardiovascular signaling, and development of the heart suggest that this gene product may be useful in treating disorders that affect the heart.

References:

Reubi J C, Gugger M, Waser B, Schaer J C. Y(1)-mediated effect of neuropeptide Y in cancer: breast carcinomas as targets. Cancer Res Jun. 1, 2001; 61(11):4636-41

Overexpression of selected peptide receptors in human tumors has been shown to represent clinically relevant targets for cancer diagnosis and therapy. Neuropeptide Y (NPY) is a peptide neurotransmitter mediating feeding behavior and vasoconstriction. It has never been shown to be involved in human cancer. We show here, using in vitro receptor autoradiography, a NPY receptor incidence of 85% in primary human breast carcinomas (n=95) and of 100% in lymph node metastases of receptor-positive primaries (n=27), predominantly as Y(1) subtype, whereas non-neoplastic human breast expressed Y(2) preferentially. Y(1) mRNA was detected in Y(1)-expressing tumors by in situ hybridization, whereas Y(2) mRNA was found in normal breast tissue. The strong predominance of Y(1) in breast carcinomas compared with Y(2) in normal breast suggests that neoplastic transformation can switch the NPY receptor expression from Y(2) to Y(1) subtype. Moreover, in Y(1)-expressing human SK-N-MC tumor cells, an NPY-induced, dose-dependent inhibition of tumor cell growth of >40% was observed, suggesting a functional role of NPY in cancer, mediated by Y(1). NPY should therefore be added to the list of small regulatory peptides related to cancer. The high incidence of Y(1) in in situ, invasive, and metastatic breast cancers allows for the possibility to target them for diagnosis and therapy with NPY analogues.

PMID: 11389101

Furtinger S, Pirker S, Czech T, Baumgartner C, Ransmayr G, Sperk G. Plasticity of Y1 and Y2 receptors and neuropeptide Y fibers in patients with temporal lobe epilepsy. J Neurosci Aug. 1, 2001;21(15):5804-12

Marked expression of neuropeptide Y (NPY) and its Y2 receptors in hippocampal mossy fibers has been reported in animal models of epilepsy. Because NPY can suppress glutamate release by activating presynaptic Y2 receptors, these changes have been proposed as an endogenous protective mechanism. Therefore, we investigated whether similar changes in the NPY system may also take place in human epilepsy. We investigated Y1 and Y2 receptor binding and NPY immunoreactivity in hippocampal specimens that were obtained at surgery from patients with temporal lobe epilepsy and in autopsy controls. Significant increases in Y2 receptor binding (by 43-48%) were observed in the dentate hilus, sectors CA1 to CA3, and subiculum of specimens with, but not in those without, hippocampal sclerosis. On the other hand, Y1 receptor binding was significantly reduced (by 62%) in the dentate molecular layer of sclerotic specimens. In the same patients, the total lengths of NPY immunoreactive (NPY-IR) fibers was markedly increased (by 115-958%) in the dentate molecular layer and hilus, in the stratum lucidum of CA3, and throughout sectors CA1 to CA3 and the subiculum, as compared with autopsies. In nonsclerotic specimens, increases in lengths of NPY-IR fibers were more moderate and statistically not significant. NPY mRNA was increased threefold in hilar interneurons of sclerotic and nonsclerotic specimens. It is suggested that abundant sprouting of NPY fibers, concomitant upregulation of Y2 receptors, and downregulation of Y1 receptors in the hippocampus of patients with Ammon's horn sclerosis may be endogenous anticonvulsant mechanisms.

PMID: 11466452

Rahmouni K, Haynes W G. Leptin signaling pathways in the central nervous system: interactions between neuropeptide Y and melanocortins. Bioessays. December 2001;23(12):1095-9.

No other hormone has drawn more attention than leptin in recent studies on the control of appetite, body weight and obesity. This hormone is produced by adipose tissue and enters the brain via a saturable specific transport mechanism. Leptin acts in the hypothalamus to modulate food intake and heat production as well as several other neuroendocrine pathways. The mechanisms through which leptin exerts its central nervous effects are now better understood. Proopiomelanocortin- and neuropeptide Y-containing neurons in the hypothalamus have emerged as potent candidate mediators of leptin action. These two neuropeptides have been shown to exert opposing effects using different pathways. Recently, Cowley et al. (2001) described a new circuit in the regulation of neuronal activity by leptin with an interaction between these two pathways. These data add complexity to the mechanisms by which leptin achieves its effects in the central nervous system, but they also offer potential mechanisms to explain the phenomenon of leptin resistance observed in obesity. Copyright 2001 John Wiley & Sons, Inc.

PMID: 11746228

Michalkiewicz M, Michalkiewicz T, Kreulen D L, McDougall S J.

Increased blood pressure responses in neuropeptide Y transgenic rats. Am J Physiol Regul Integr Comp Physiol August 2001;281(2):R417-26

Considering the coexistence of neuropeptide Y (NPY) and norepinephrine in perivascular sympathetic nerves and the known vasoconstrictor cooperation of NPY with norepinephrine, we investigated the involvement of NPY in long-term control of cardiovascular functions using NPY transgenic (NPY-tg) rats. These rats were developed by injection of the rat (Sprague-Dawley) pronuclei with a 14.5-kb clone of the rat structural NPY gene. When compared with nontransgenic littermates, NPY concentrations were significantly increased in a number of cardiovascular tissues of NPY-tg hemizygotes. Direct basal mean arterial pressure and heart rate were not changed, but calculated total vascular resistance was significantly increased in NPY-tg subjects. Arterial pressure increases, in response to norepinephrine injection, were greater in the NPY-tg rats. Also, the hypotension and bradycardia in response to hemorrhage were significantly reduced in NPY-tg subjects. These results indicate that NPY, when expressed in increased amounts, potentiates the pressor effects of norepinephrine and contributes to maintaining blood pressure during hemorrhage, but it does not alter resting blood pressure. These transgenic rats will facilitate studies of the role of NPY signaling in cardiovascular regulation, particularly regarding its functional cooperation with norepinephrine.

PMID: 11448843

Horackova M, Slavikova J, Byczko Z. Postnatal development of the rat intrinsic cardiac nervous system: a confocal laser scanning microscopy study in whole-mount atria. Tissue Cell October 2000;32(5):377-88

We used confocal laser scanning microscopy and fluorescent immunohistochemistry to study the developmental pattern and distribution of specific neuronal phenotypes within the intrinsic cardiac nervous system in whole-mount atrial preparations from newborn to 5 week old rats. Individual ganglia and neuronal cell bodies were localized by means of two general neuronal markers: protein gene product 9.5 (PGP) and microtubule-associated protein two (MAP). In rats < or=2 weeks old there were two main subpopulations of intrinsic neurons located in the intraatrial septum and around the origin of the superior vena cava. The more abundant was a population of strongly tyrosine hydroxylase (TH) immunoreactive (IR) neurons (10-40 microm in diameter) most of which were also PGP-IR. The second, less numerous (approximately 60-70% than the TH-IR group) type of neurons exhibited ChAT-IR which colocalized with MAP-IR. Towards the end of the second postnatal week and during the third, the ganglia containing these neurons became more numerous and their localization also included tissues around the origins of the inferior vena cava and the pulmonary veins, as well as both atrial walls close to the AV junction. During the second and third postnatal weeks, when the extrinsic innervation of the adrenergic and cholinergic phenotypes largely increases, the intrinsic innervation also changed greatly, and around the 21st postnatal day it appeared to acquire mature characteristics. The TH-IR neurons changed their characteristics and formed two types of ganglia. The larger ganglia containing large cells (20-40 microm in diameter) expressed TH-IR mostly close to their inner body surface (approximately 80-90% of identified neurons). Most of these neurons also expressed neuropeptide Y (NPY)-IR, specifically around their nuclei. The second type of small strongly TH-IR neurons (approximately 10% of all identified neurons) were contained in smaller groups (20-50 cells) which were usually embedded into much larger ganglia (100-400 cells), containing large (20-50 microm) neurons. Unlike all other intrinsic neurons, these small TH-IR cells did not exhibit any PGP-IR or MAP-IR. The number of ChAT-IR neurons increased at this stage, reaching approximately 90% of the neurons identified by, the general neuronal markers. These neurons were surrounded by a rich network of cholinergic varicose nerve fibers, some of which were likely of an extrinsic origin. We have also identified relatively small ganglia expressing immunoreactivity to vasoactive intestinal polypeptide (VIP), and to substance P (SP). The presented data indicate that the phenotypes of intrinsic neurons in the rat heart change greatly during the first month of postnatal development. This may be at least partially related to the development and maturation of functional extrinsic nervous control of the heart.

PMID: 11201277

Panel 1.3D Summary: Ag2930 The expression of the NOV9a gene was assessed in two independent runs on this panel. Low but significant levels of expression are seen in kidney cancer cells and a lung cancer cell, consistent with Panel 1.1. Please see the previous panel for discussion of utility of this gene in cancer.

The expression in this panel also confirms expression of this gene product in the brain. Please see Panel 1.1 for discussion of utility of this gene in the central nervous system.

Panel 2D Summary: Ag2930 The NOV9a gene is expressed at low but significant levels in kidney cancer samples in this panel but not in the adjacent normal tissue samples (CTs=30-32). This expression is consistent with results in the preceding panels. This suggests that expression of this gene can be used as a diagnostic marker for the presence of kidney cancer. Furthermore, therapeutic inhibition of the gene product could potentially be used in the treatment of kidney cancer.

Panel 3D Summary: Ag2930 The NOV9a gene expression is restricted to NCI-N417, a small cell lung cancer cell line (CT=33.81). Expression of this gene can therefore be used for the diagnosis and treatment of this cancer.

Panel 4D Summary: Ag2930 Expression of the NOV9a gene is low/undetectable in all samples on this panel. (CTs>35). (Data not shown.)

NOV4b

Expression of gene NOV4b was assessed using the primer-probe set Ag2955, described in Table BA. Results of the RTQ-PCR runs are shown in Tables BB, BC and BD.

TABLE BA
Probe Name Ag2955

| | | | Start | |
|---|---|---|---|---|
| Primers | Sequences | Length | Position | SEQ ID NO: |
| Forward | 5′-caatgcaatctgttggactttt-3′ | 22 | 33 | 1003 |
| Probe | TET-5′-tctctcctgggatggattttatccat-3′-TAMRA | 26 | 59 | 1004 |
| Reverse | 5′-gttcttccagtgtggcaaataa-3′ | 22 | 94 | 1005 |

TABLE BB
General_screening_panel_v1.4

| | Rel. Exp. (%) Ag2955, | | Rel. Exp. (%) Ag2955, |
|---|---|---|---|
| Tissue Name | Run 216860933 | Tissue Name | Run 216860933 |
| Adipose | 8.1 | Renal ca. TK-10 | 8.0 |
| Melanoma* | 8.5 | Bladder | 18.9 |
| Hs688(A).T | | | |
| Melanoma* | 5.8 | Gastric ca. (liver met.) | 35.6 |
| Hs688(B).T | | NCI-N87 | |
| Melanoma* M14 | 1.1 | Gastric ca. KATO III | 2.5 |
| Melanoma* | 31.0 | Colon ca. SW-948 | 0.0 |
| LOXIMVI | | | |
| Melanoma* SK- | 3.1 | Colon ca. SW480 | 4.4 |
| MEL-5 | | | |
| Squamous cell | 1.3 | Colon ca.* (SW480 | 1.7 |
| carcinoma SCC-4 | | met) SW620 | |
| Testis Pool | 0.0 | Colon ca. HT29 | 3.9 |
| Prostate ca.* (bone | 36.9 | Colon HCT-116 | 5.0 |
| met) PC-3 | | | |
| Prostate Pool | 0.0 | Colon ca. CaCo-2 | 5.9 |
| Placenta | 0.0 | Colon cancer tissue | 0.0 |
| Uterus Pool | 0.0 | Colon ca. SW1116 | 0.0 |
| Ovarian ca. | 10.5 | Colon ca. Colo-205 | 3.3 |
| OVCAR-3 | | | |
| Ovarian ca. SK-OV-3 | 47.3 | Colon ca. SW-48 | 0.0 |
| Ovarian ca. | 4.5 | Colon Pool | 1.6 |
| OVCAR-4 | | | |
| Ovarian ca. | 6.5 | Small Intestine Pool | 2.4 |
| OVCAR-5 | | | |
| Ovarian ca. IGROV-1 | 21.0 | Stomach Pool | 4.3 |
| Ovarian ca. | 3.1 | Bone Marrow Pool | 5.3 |
| OVCAR-8 | | | |
| Ovary | 4.3 | Fetal Heart | 2.9 |
| Breast ca. MCF-7 | 0.0 | Heart Pool | 3.8 |
| Breast ca. MDA- | 29.7 | Lymph Node Pool | 8.0 |
| MB-231 | | | |
| Breast ca. BT 549 | 3.0 | Fetal Skeletal Muscle | 1.6 |
| Breast ca. T47D | 6.3 | Skeletal Muscle Pool | 7.6 |
| Breast ca. MDA-N | 1.6 | Spleen Pool | 2.4 |
| Breast Pool | 0.7 | Thymus Pool | 1.2 |
| Trachea | 2.5 | CNS cancer (glio/astro) | 2.0 |
| | | U87-MG | |
| Lung | 1.5 | CNS cancer (glio/astro) | 1.2 |
| | | U-118-MG | |
| Fetal Lung | 9.6 | CNS cancer | 5.3 |
| | | (neuro; met) SK-N-AS | |
| Lung ca. NCI-N417 | 0.0 | CNS cancer (astro) SF- | 2.6 |
| | | 539 | |
| Lung ca. LX-1 | 2.4 | CNS cancer (astro) | 6.7 |
| | | SNB-75 | |
| Lung ca. NCI-H146 | 0.0 | CNS cancer (glio) | 9.3 |
| | | SNB-19 | |
| Lung ca. SHP-77 | 1.7 | CNS cancer (glio) SF- | 62.9 |
| | | 295 | |
| Lung ca. A549 | 3.0 | Brain (Amygdala) Pool | 1.3 |
| Lung ca. NCI-H526 | 0.0 | Brain (cerebellum) | 4.0 |
| Lung ca. NCI-H23 | 59.0 | Brain (fetal) | 7.2 |
| Lung ca. NCI-H460 | 2.1 | Brain (Hippocampus) | 2.1 |
| | | Pool | |
| Lung ca. HOP-62 | 3.0 | Cerebral Cortex Pool | 3.0 |
| Lung ca. NCI-H522 | 65.1 | Brain (Substantia nigra) | 0.6 |
| | | Pool | |
| Liver | 0.0 | Brain (Thalamus) Pool | 2.6 |
| Fetal Liver | 3.1 | Brain (whole) | 0.0 |
| Liver ca. HepG2 | 4.1 | Spinal Cord Pool | 0.0 |
| Kidney Pool | 4.4 | Adrenal Gland | 0.0 |
| Fetal Kidney | 23.2 | Pituitary gland Pool | 1.0 |
| Renal ca. 786-0 | 2.5 | Salivary Gland | 0.0 |
| Renal ca. A498 | 1.9 | Thyroid (female) | 1.7 |
| Renal ca. ACHN | 2.7 | Pancreatic ca. CAPAN2 | 100.0 |
| Renal ca. UO-31 | 1.7 | Pancreas Pool | 6.5 |

TABLE BC
Panel 1.3D

| | Rel. | | Rel. |
|---|---|---|---|
| | Exp. (%) | | Exp. (%) |
| | Ag2955, | | Ag2955, |
| | Run | | Run |
| Tissue Name | 167906363 | Tissue Name | 167906363 |
| Liver | 8.5 | Kidney (fetal) | 4.4 |
| adenocarcinoma | | | |
| Pancreas | 0.0 | Renal ca. 786-0 | 3.2 |
| Pancreatic ca. | 64.6 | Renal ca. A498 | 15.1 |
| CAPAN2 | | | |
| Adrenal gland | 0.0 | Renal ca. RXF 393 | 10.7 |
| Thyroid | 1.5 | Renal ca. ACHN | 6.5 |
| Salivary gland | 1.1 | Renal ca. UO-31 | 2.3 |
| Pituitary gland | 0.0 | Renal ca. TK-10 | 13.2 |
| Brain (fetal) | 0.0 | Liver | 0.0 |
| Brain (whole) | 2.7 | Liver (fetal) | 0.0 |
| Brain (amygdala) | 12.1 | Liver ca. | 8.5 |
| | | (hepatoblast) HepG2 | |
| Brain (cerebellum) | 1.0 | Lung | 6.4 |
| Brain | 0.0 | Lung (fetal) | 2.9 |
| (hippocampus) | | | |
| Brain (substantia | 6.2 | Lung ca. (small cell) | 5.5 |
| nigra) | | LX-1 | |
| Brain (thalamus) | 0.0 | Lung ca. (small cell) | 0.0 |
| | | NCI-H69 | |
| Cerebral Cortex | 3.9 | Lung ca. (s.cell var.) | 4.1 |
| | | SHP-77 | |
| Spinal cord | 1.9 | Lung ca. (large | 1.1 |
| | | cell)NCI-H460 | |
| glio/astro U87-MG | 0.0 | Lung ca. (non-sm. | 19.2 |
| | | cell) A549 | |
| glio/astro | 0.0 | Lung ca. (non-s.cell) | 26.1 |
| U-118-MG | | NCI-H23 | |
| astrocytoma | 7.6 | Lung ca. (non-s.cell) | 12.3 |
| SW1783 | | HOP-62 | |
| neuro*; met | 3.5 | Lung ca. (non-s.cl) | 46.3 |
| SK-N-AS | | NCI-H522 | |
| astrocytoma SF-539 | 13.2 | Lung ca. (squam.) | 16.7 |
| | | SW 900 | |
| astrocytoma | 3.6 | Lung ca. (squam.) | 2.8 |
| SNB-75 | | NCI-H596 | |
| glioma SNB-19 | 16.5 | Mammary gland | 0.0 |
| glioma U251 | 26.6 | Breast ca.* (pl.ef) | 3.0 |
| | | MCF-7 | |
| glioma SF-295 | 6.0 | Breast ca.* (pl.ef) | 7.1 |
| | | MDA-MB-231 | |
| Heart (fetal) | 0.0 | Breast ca.* (pl.ef) | 11.5 |
| | | T47D | |
| Heart | 1.1 | Breast ca. BT-549 | 1.1 |
| Skeletal muscle | 3.3 | Breast ca. MDA-N | 5.7 |
| (fetal) | | | |
| Skeletal muscle | 0.0 | Ovary | 0.0 |
| Bone marrow | 0.0 | Ovarian ca. OVCAR-3 | 21.2 |
| Thymus | 0.0 | Ovarian ca. OVCAR-4 | 7.4 |
| Spleen | 0.0 | Ovarian ca. OVCAR-5 | 14.3 |
| Lymph node | 0.0 | Ovarian ca. OVCAR-8 | 4.7 |
| Colorectal | 1.4 | Ovarian ca. IGROV-1 | 38.4 |
| Stomach | 0.9 | Ovarian ca.* (ascites) | 100.0 |
| | | SK-OV-3 | |
| Small intestine | 0.0 | Uterus | 0.0 |
| Colon ca. SW480 | 6.8 | Placenta | 0.0 |
| Colon ca.* | 7.6 | Prostate | 0.0 |
| SW620(SW480 | | | |
| met) | | | |
| Colon ca. HT29 | 6.1 | Prostate ca.* (bone | 13.1 |
| | | met)PC-3 | |
| Colon ca. HCT-116 | 4.2 | Testis | 0.0 |
| Colon ca. CaCo-2 | 19.3 | Melanoma | 14.7 |
| | | Hs688(A).T | |
| Colon ca. | 0.0 | Melanoma* (met) | 5.4 |
| tissue(ODO3866) | | Hs688(B).T | |
| Colon ca. | 3.6 | Melanoma UACC-62 | 0.0 |
| HCC-2998 | | | |
| Gastric ca.* | 20.6 | Melanoma M14 | 0.0 |
| (liver met) | | | |
| NCI-N87 | | | |
| Bladder | 13.4 | Melanoma LOX | 25.5 |
| | | IMVI | |
| Trachea | 3.6 | Melanoma* (met) | 1.7 |
| | | SK-MEL-5 | |
| Kidney | 5.1 | Adipose | 13.9 |

TABLE BD
Panel 4D

| | Rel. Exp. (%) | | Rel. Exp. (%) |
|---|---|---|---|
| | Ag2955, Run | | Ag2955, Run |
| Tissue Name | 164306318 | Tissue Name | 164306318 |
| Secondary Th1 act | 0.0 | HUVEC IL-1beta | 6.6 |
| Secondary Th2 act | 0.0 | HUVEC IFN gamma | 20.2 |
| Secondary Tr1 act | 0.0 | HUVEC TNF alpha + IFN | 15.7 |
| | | gamma | |
| Secondary Th1 rest | 0.0 | HUVEC TNF alpha + IL4 | 8.4 |
| Secondary Th2 rest | 0.0 | HUVEC IL-11 | 0.0 |
| Secondary Tr1 rest | 0.0 | Lung Microvascular EC | 6.0 |
| | | none | |
| Primary Th1 act | 0.0 | Lung Microvascular EC | 8.7 |
| | | TNF alpha + IL-1beta | |
| Primary Th2 act | 0.0 | Microvascular Dermal EC | 7.7 |
| | | none | |
| Primary Tr1 act | 0.0 | Microsvasular Dermal EC | 6.4 |
| | | TNF alpha + IL-1beta | |
| Primary Th1 rest | 0.0 | Bronchial epithelium | 9.5 |
| | | TNF alpha + IL1beta | |
| Primary Th2 rest | 0.0 | Small airway epithelium | 5.3 |
| | | none | |
| Primary Tr1 rest | 0.0 | Small airway epithelium | 19.9 |
| | | TNF alpha + IL-1beta | |
| CD45RA CD4 | 13.5 | Coronery artery SMC rest | 22.4 |
| lymphocyte act | | | |
| CD45RO CD4 | 0.0 | Coronery artery SMC | 12.0 |
| lymphocyte act | | TNF alpha + IL-1beta | |
| CD8 lymphocyte act | 0.0 | Astrocytes rest | 37.6 |
| Secondary CD8 | 0.0 | Astrocytes TNF alpha + | 29.3 |
| lymphocyte act | | IL-1beta | |
| Secondary CD8 | 0.0 | KU-812 (Basophil) rest | 25.7 |
| lymphocyte act | | | |
| CD4 lymphocyte none | 0.0 | KU-812 (Basophil) | 100.0 |
| | | PMA/ionomycin | |
| 2ry Th1/Th2/Tr1_anti- | 0.0 | CCD1106 (Keratinocytes) | 4.2 |
| CD95 CH11 | | none | |
| LAK cells rest | 0.0 | CCD1106 (Keratinocytes) | 1.3 |
| | | TNF alpha + IL-1beta | |
| LAK cells IL-2 | 0.0 | Liver cirrhosis | 7.5 |
| LAK cells IL-2 + IL-12 | 0.0 | Lupus kidney | 5.0 |
| LAK cells IL-2 + IFN | 0.0 | NCI-H292 none | 31.9 |
| gamma | | | |
| LAK cells IL-2 + IL-18 | 0.0 | NCI-H292 IL-4 | 52.1 |
| LAK cells | 0.0 | NCI-H292 IL-9 | 39.5 |
| PMA/ionomycin | | | |
| NK Cells IL-2 rest | 0.0 | NCI-H292 IL-13 | 16.6 |
| Two Way MLR 3 day | 3.9 | NCI-H292 IFN gamma | 35.4 |
| Two Way MLR 5 day | 0.0 | HPAEC none | 4.0 |
| Two Way MLR 7 day | 0.0 | HPAEC TNF alpha + IL- | 3.0 |
| | | 1beta | |
| PBMC rest | 0.0 | Lung fibroblast none | 4.9 |
| PBMC PWM | 0.0 | Lung fibroblast TNF alpha + | 0.0 |
| | | IL-1beta | |
| PBMC PHA-L | 5.5 | Lung fibroblast IL-4 | 9.9 |
| Ramos (B cell) none | 0.0 | Lung fibroblast IL-9 | 6.3 |
| Ramos (B cell) | 0.0 | Lung fibroblast IL-13 | 1.7 |
| ionomycin | | | |
| B lymphocytes PWM | 0.0 | Lung fibroblast IFN | 11.7 |
| | | gamma | |
| B lymphocytes CD40L | 0.0 | Dermal fibroblast | 59.9 |
| and IL-4 | | CCD1070 rest | |
| EOL-1 dbcAMP | 0.0 | Dermal fibroblast | 80.7 |
| | | CCD1070 TNF alpha | |
| EOL-1 dbcAMP | 0.0 | Dermal fibroblast | 35.4 |
| PMA/ionomycin | | CCD1070 IL-1beta | |
| Dendritic cells none | 10.6 | Dermal fibroblast IFN | 70.2 |
| | | gamma | |
| Dendritic cells LPS | 4.5 | Dermal fibroblast IL-4 | 72.7 |
| Dendritic cells anti- | 0.0 | IBD Colitis 2 | 4.2 |
| CD40 | | | |
| Monocytes rest | 7.2 | IBD Crohn's | 0.0 |
| Monocytes LPS | 2.4 | Colon | 6.7 |
| Macrophages rest | 3.3 | Lung | 22.4 |
| Macrophages LPS | 0.0 | Thymus | 23.2 |
| HUVEC none | 7.3 | Kidney | 0.0 |
| HUVEC starved | 19.6 | | |

General_screening_panel_v1.4 Summary: Ag2955 Highest expression of the NOV4b gene is seen in a pancreatic cancer cell line (CT=32.6). Low but significant levels of expression are also seen in melanoma, lung, brain, ovarian, breast and prostate cancer cell lines. Thus, expression of this gene might be used as a diagnostic marker for the presence of these cancers. Furthermore, therapeutic inhibition of this gene product may be useful in the treatment of melanoma, lung, brain, ovarian, breast and prostate cancers.

Panel 1.3D Summary: Ag2955 Highest expression of the NOV4b gene is seen in an ovarian cancer cell line, SK-OV-3, (CT=33.1). Low but significant levels of expression are also seen in melanoma, lung, brain and pancreatic cancer cell lines. Thus, expression of this gene might be used as a diagnostic marker for the presence of these cancers. Furthermore, therapeutic inhibition of this gene product may be useful in the treatment of melanoma, lung, brain, and pancreatic cancers.

Panel 4D Summary: Ag 2955 The NOV4b gene is expressed at low but significant levels in treated and untreated dermal fibroblasts and in the basophil cell line treated with PMA and ionomycin. The latter mimics the condition that leads to the degranulation and release of various mediators which contribute to the symptomatology of allergic diseases. This transcript encodes a claudin 6 like protein, a member of the Claudin tight junction family. The expression of this transcript could potentially be used as a marker for activated basophils and dermal fibroblasts. Furthermore, modulation of the activity or expression of this putative protein by antibodies may reduce the symptoms of patients suffering from allergic diseases asthma, ulcerative colitis, atopic diseases such as contact dermatitis and eczema, or inflammatory skin diseases.

NOV3b

Expression of gene NOV3b was assessed using the primer-probe set Ag2957, described in Table CA. Results of the RTQ-PCR runs are shown in Tables CB, CC, CD and CE.

TABLE CA
Probe Name Ag2957

| | | | Start | |
|---|---|---|---|---|
| Primers | Sequences | Length | Position | SEQ ID NO: |
| Forward | 5′-caggcctctgcactttgac-3′ | 19 | 438 | 1006 |
| Probe | TET-5′-ctgtctcgtggtatgccaccctggt-3′-TAMRA | 25 | 459 | 1007 |
| Reverse | 5′-ccaaattctgggttgaagaact-3′ | 22 | 492 | 1008 |

TABLE CB
General_screening_panel_v1.4

| | Rel. Exp. (%) Ag2957, | | Rel. Exp. (%) Ag2957, |
|---|---|---|---|
| Tissue Name | Run 216861284 | Tissue Name | Run 216861284 |
| Adipose | 1.8 | Renal ca. TK-10 | 6.0 |
| Melanoma* | 0.0 | Bladder | 0.0 |
| Hs688(A).T | | | |
| Melanoma* | 0.0 | Gastric ca. (liver met.) | 0.0 |
| Hs688(B).T | | NCI-N87 | |
| Melanoma* M14 | 0.0 | Gastric ca. KATO III | 0.0 |
| Melanoma* | 0.0 | Colon ca. SW-948 | 0.0 |
| LOXIMVI | | | |
| Melanoma* SK- | 0.0 | Colon ca. SW480 | 0.0 |
| MEL-5 | | | |
| Squamous cell | 0.0 | Colon ca.* (SW480 | 4.6 |
| carcinoma SCC-4 | | met) SW620 | |
| Testis Pool | 0.0 | Colon ca. HT29 | 0.0 |
| Prostate ca.* (bone | 0.0 | Colon ca. HCT-116 | 0.0 |
| met) PC-3 | | | |
| Prostate Pool | 0.0 | Colon ca. CaCo-2 | 24.8 |
| Placenta | 41.5 | Colon cancer tissue | 1.4 |
| Uterus Pool | 0.0 | Colon ca. SW1116 | 0.0 |
| Ovarian ca. | 0.0 | Colon ca. Colo-205 | 0.0 |
| OVCAR-3 | | | |
| Ovarian ca. SK-OV-3 | 2.7 | Colon ca. SW-48 | 0.0 |
| Ovarian ca. | 0.0 | Colon Pool | 0.0 |
| OVCAR-4 | | | |
| Ovarian ca. | 0.0 | Small Intestine Pool | 0.0 |
| OVCAR-5 | | | |
| Ovarian ca. IGROV-1 | 0.0 | Stomach Pool | 0.0 |
| Ovarian ca. | 0.0 | Bone Marrow Pool | 0.0 |
| OVCAR-8 | | | |
| Ovary | 0.0 | Fetal Heart | 0.0 |
| Breast ca. MCF-7 | 0.0 | Heart Pool | 0.0 |
| Breast ca. MDA- | 0.0 | Lymph Node Pool | 0.0 |
| MB-231 | | | |
| Breast ca. BT 549 | 1.7 | Fetal Skeletal Muscle | 8.0 |
| Breast ca. T47D | 0.0 | Skeletal Muscle Pool | 0.0 |
| Breast ca. MDA-N | 0.0 | Spleen Pool | 0.0 |
| Breast Pool | 0.0 | Thymus Pool | 3.0 |
| Trachea | 2.8 | CNS cancer (glio/astro) | 0.0 |
| | | U87-MG | |
| Lung | 2.9 | CNS cancer (glio/astro) | 0.0 |
| | | U-118-MG | |
| Fetal Lung | 24.5 | CNS cancer | 0.0 |
| | | (neuro; met) SK-N-AS | |
| Lung ca. NCI-N417 | 0.0 | CNS cancer (astro) SF- | 0.0 |
| | | 539 | |
| Lung ca. LX-1 | 0.9 | CNS cancer (astro) | 0.0 |
| | | SNB-75 | |
| Lung ca. NCI-H146 | 0.0 | CNS cancer (glio) | 0.0 |
| | | SNB-19 | |
| Lung ca. SHP-77 | 0.0 | CNS cancer (glio) SF- | 0.0 |
| | | 295 | |
| Lung ca. A549 | 0.0 | Brain (Amygdala) Pool | 0.0 |
| Lung ca. NCI-H526 | 6.0 | Brain (cerebellum) | 0.0 |
| Lung ca. NCI-H23 | 0.0 | Brain (fetal) | 1.7 |
| Lung ca. NCI-H460 | 0.0 | Brain (Hippocampus) | 4.7 |
| | | Pool | |
| Lung ca. HOP-62 | 0.0 | Cerebral Cortex Pool | 0.0 |
| Lung ca. NCI-H522 | 0.0 | Brain (Substantia nigra) | 0.0 |
| | | Pool | |
| Liver | 0.0 | Brain (Thalamus) Pool | 0.0 |
| Fetal Liver | 31.4 | Brain (whole) | 0.0 |
| Liver ca. HepG2 | 7.6 | Spinal Cord Pool | 22.4 |
| Kidney Pool | 1.7 | Adrenal Gland | 0.0 |
| Fetal Kidney | 100.0 | Pituitary gland Pool | 0.0 |
| Renal ca. 786-0 | 0.0 | Salivary Gland | 0.0 |
| Renal ca. A498 | 0.0 | Thyroid (female) | 0.0 |
| Renal ca. ACHN | 0.0 | Pancreatic ca. CAPAN2 | 2.0 |
| Renal ca. UO-31 | 0.0 | Pancreas Pool | 0.0 |

TABLE CC
Panel 1.3D

| | Rel. Exp. (%) Ag2957, | | Rel. Exp. (%) Ag2957, |
|---|---|---|---|
| Tissue Name | Run 167906390 | Tissue Name | Run 167906390 |
| Liver adenocarcinoma | 0.0 | Kidney (fetal) | 34.2 |
| Pancreas | 0.0 | Renal ca. 786-0 | 0.0 |
| Pancreatic ca. CAPAN2 | 0.1 | Renal ca. A498 | 0.0 |
| Adrenal gland | 0.0 | Renal ca. RXF 393 | 0.0 |
| Thyroid | 0.0 | Renal ca. ACHN | 0.0 |
| Salivary gland | 0.2 | Renal ca. UO-31 | 0.0 |
| Pituitary gland | 0.0 | Renal ca. TK-10 | 0.0 |
| Brain (fetal) | 0.0 | Liver | 100.0 |
| Brain (whole) | 0.0 | Liver (fetal) | 2.6 |
| Brain (amygdala) | 0.0 | Liver ca. | 2.7 |
| | | (hepatoblast) HepG2 | |
| Brain (cerebellum) | 0.0 | Lung | 0.0 |
| Brain (hippocampus) | 0.2 | Lung (fetal) | 0.8 |
| Brain (substantia nigra) | 0.3 | Lung ca. (small cell) | 0.0 |
| | | LX-1 | |
| Brain (thalamus) | 0.0 | Lung ca. (small cell) | 0.0 |
| | | NCI-H69 | |
| Cerebral Cortex | 0.0 | Lung ca. (s.cell var.) | 1.5 |
| | | SHP-77 | |
| Spinal cord | 23.8 | Lung ca. (large | 0.0 |
| | | cell)NCI-H460 | |
| glio/astro U87-MG | 0.0 | Lung ca. (non-sm. | 0.0 |
| | | cell) A549 | |
| glio/astro U-118-MG | 0.0 | Lung ca. (non-s.cell) | 0.3 |
| | | NCI-H23 | |
| astrocytoma SW1783 | 0.0 | Lung ca. (non-s.cell) | 0.0 |
| | | HOP-62 | |
| neuro*; met SK-N-AS | 0.0 | Lung ca. (non-s.cl) | 0.0 |
| | | NCI-H522 | |
| astrocytoma SF-539 | 0.2 | Lung ca. (squam.) | 2.1 |
| | | SW 900 | |
| astrocytoma SNB-75 | 0.1 | Lung ca. (squam.) | 0.0 |
| | | NCI-H596 | |
| glioma SNB-19 | 0.0 | Mammary gland | 1.7 |
| glioma U251 | 0.0 | Breast ca.* (pl.ef) | 0.0 |
| | | MCF-7 | |
| glioma SF-295 | 0.0 | Breast ca.* (pl.ef) | 0.0 |
| | | MDA-MB-231 | |
| Heart (fetal) | 0.0 | Breast ca.* (pl.ef) | 0.0 |
| | | T47D | |
| Heart | 0.6 | Breast ca. BT-549 | 0.3 |
| Skeletal muscle (fetal) | 2.2 | Breast ca. MDA-N | 0.0 |
| Skeletal muscle | 1.5 | Ovary | 0.0 |
| Bone marrow | 0.0 | Ovarian ca. OVCAR-3 | 0.0 |
| Thymus | 2.3 | Ovarian ca. OVCAR-4 | 0.0 |
| Spleen | 0.0 | Ovarian ca. OVCAR-5 | 0.0 |
| Lymph node | 0.1 | Ovarian ca. OVCAR-8 | 0.0 |
| Colorectal | 0.0 | Ovarian ca. IGROV-1 | 0.0 |
| Stomach | 0.0 | Ovarian ca.* (ascites) | 0.1 |
| | | SK-OV-3 | |
| Small intestine | 0.0 | Uterus | 0.0 |
| Colon ca. SW480 | 0.0 | Placenta | 0.8 |
| Colon ca.* | 0.3 | Prostate | 0.0 |
| SW620(SW480 met) | | | |
| Colon ca. HT29 | 0.0 | Prostate ca.* (bone | 0.0 |
| | | met)PC-3 | |
| Colon ca. HCT-116 | 0.0 | Testis | 0.0 |
| Colon ca. CaCo-2 | 2.2 | Melanoma | 0.0 |
| | | Hs688(A).T | |
| Colon ca. | 0.0 | Melanoma* (met) | 0.0 |
| tissue(ODO3866) | | Hs688(B).T | |
| Colon ca. HCC-2998 | 0.0 | Melanoma UACC-62 | 0.0 |
| Gastric ca.* (liver met) | 0.0 | Melanoma M14 | 0.0 |
| NCI-N87 | | | |
| Bladder | 0.0 | Melanoma LOX | 0.1 |
| | | IMVI | |
| Trachea | 0.2 | Melanoma* (met) | 0.0 |
| | | SK-MEL-5 | |
| Kidney | 30.6 | Adipose | 0.3 |

TABLE CD
Panel 2D

| | Rel. Exp. (%) | | Rel. Exp. (%) |
|---|---|---|---|
| | Ag2957, | | Ag2957, |
| Tissue Name | Run 170858345 | Tissue Name | Run 170858345 |
| Normal Colon | 0.0 | Kidney Margin | 68.8 |
| | | 8120608 | |
| CC Well to Mod Diff | 1.0 | Kidney Cancer | 0.0 |
| (ODO3866) | | 8120613 | |
| CC Margin (ODO3866) | 0.0 | Kidney Margin | 24.3 |
| | | 8120614 | |
| CC Gr.2 rectosigmoid | 13.6 | Kidney Cancer | 3.4 |
| (ODO3868) | | 9010320 | |
| CC Margin (ODO3868) | 1.9 | Kidney Margin | 85.9 |
| | | 9010321 | |
| CC Mod Diff (ODO3920) | 0.0 | Normal Uterus | 0.0 |
| CC Margin (ODO3920) | 0.0 | Uterus Cancer 064011 | 1.6 |
| CC Gr.2 ascend colon | 0.0 | Normal Thyroid | 0.0 |
| (ODO3921) | | | |
| CC Margin (ODO3921) | 1.2 | Thyroid Cancer | 0.0 |
| | | 064010 | |
| CC from Partial | 0.0 | Thyroid Cancer | 0.0 |
| Hepatectomy (ODO4309) | | A302152 | |
| Mets | | | |
| Liver Margin (ODO4309) | 0.0 | Thyroid Margin | 0.0 |
| | | A302153 | |
| Colon mets to lung | 0.0 | Normal Breast | 3.3 |
| (OD04451-01) | | | |
| Lung Margin (OD04451- | 0.0 | Breast Cancer | 0.0 |
| 02) | | (OD04566) | |
| Normal Prostate 6546-1 | 1.3 | Breast Cancer | 3.0 |
| | | (OD04590-01) | |
| Prostate Cancer | 0.0 | Breast Cancer Mets | 0.0 |
| (OD04410) | | (OD04590-03) | |
| Prostate Margin | 2.6 | Breast Cancer | 0.0 |
| (OD04410) | | Metastasis | |
| | | (OD04655-05) | |
| Prostate Cancer | 0.0 | Breast Cancer 064006 | 0.0 |
| (OD04720-01) | | | |
| Prostate Margin | 0.0 | Breast Cancer 1024 | 9.5 |
| (OD04720-02) | | | |
| Normal Lung 061010 | 0.0 | Breast Cancer | 3.3 |
| | | 9100266 | |
| Lung Met to Muscle | 0.0 | Breast Margin | 5.4 |
| (ODO4286) | | 9100265 | |
| Muscle Margin | 0.0 | Breast Cancer | 4.1 |
| (ODO4286) | | A209073 | |
| Lung Malignant Cancer | 0.0 | Breast Margin | 4.9 |
| (OD03126) | | A2090734 | |
| Lung Margin (OD03126) | 0.0 | Normal Liver | 0.0 |
| Lung Cancer (OD04404) | 0.0 | Liver Cancer 064003 | 0.0 |
| Lung Margin (OD04404) | 0.0 | Liver Cancer 1025 | 0.0 |
| Lung Cancer (OD04565) | 0.0 | Liver Cancer 1026 | 0.0 |
| Lung Margin (OD04565) | 0.0 | Liver Cancer 6004-T | 0.0 |
| Lung Cancer (OD04237- | 0.0 | Liver Tissue 6004-N | 12.4 |
| 01) | | | |
| Lung Margin (OD04237- | 0.0 | Liver Cancer 6005-T | 5.7 |
| 02) | | | |
| Ocular Mel Met to Liver | 0.0 | Liver Tissue 6005-N | 0.0 |
| (ODO4310) | | | |
| Liver Margin (ODO4310) | 1.4 | Normal Bladder | 0.0 |
| Melanoma Mets to Lung | 0.0 | Bladder Cancer 1023 | 0.0 |
| (OD04321) | | | |
| Lung Margin (OD04321) | 0.0 | Bladder Cancer | 0.0 |
| | | A302173 | |
| Normal Kidney | 85.3 | Bladder Cancer | 0.0 |
| | | (OD04718-01) | |
| Kidney Ca, Nuclear grade | 6.2 | Bladder Normal | 0.0 |
| 2 (OD04338) | | Adjacent (OD04718- | |
| | | 03) | |
| Kidney Margin | 32.3 | Normal Ovary | 0.0 |
| (OD04338) | | | |
| Kidney Ca Nuclear grade | 13.5 | Ovarian Cancer | 0.0 |
| 1/2 (OD04339) | | 064008 | |
| Kidney Margin | 92.0 | Ovarian Cancer | 28.7 |
| (OD04339) | | (OD04768-07) | |
| Kidney Ca, Clear cell | 9.7 | Ovary Margin | 0.0 |
| type (OD04340) | | (OD04768-08) | |
| Kidney Margin | 87.7 | Normal Stomach | 0.0 |
| (OD04340) | | | |
| Kidney Ca, Nuclear grade | 0.0 | Gastric Cancer | 0.0 |
| 3 (OD04348) | | 9060358 | |
| Kidney Margin | 100.0 | Stomach Margin | 0.0 |
| (OD04348) | | 9060359 | |
| Kidney Cancer | 0.0 | Gastric Cancer | 0.0 |
| (OD04622-01) | | 9060395 | |
| Kidney Margin | 50.3 | Stomach Margin | 0.0 |
| (OD04622-03) | | 9060394 | |
| Kidney Cancer | 0.0 | Gastric Cancer | 0.0 |
| (OD04450-01) | | 9060397 | |
| Kidney Margin | 58.6 | Stomach Margin | 0.0 |
| (OD04450-03) | | 9060396 | |
| Kidney Cancer 8120607 | 0.0 | Gastric Cancer | 0.0 |
| | | 064005 | |

TABLE CE
Panel 4D

| | Rel. Exp. (%) | | Rel. Exp. (%) |
|---|---|---|---|
| | Ag2957, Run | | Ag2957, Run |
| Tissue Name | 164306319 | Tissue Name | 164306319 |
| Secondary Th1 act | 0.0 | HUVEC IL-1 beta | 0.0 |
| Secondary Th2 act | 0.8 | HUVEC IFN gamma | 0.0 |
| Secondary Tr1 act | 0.0 | HUVEC TNF alpha + IFN | 0.0 |
| | | gamma | |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC | 0.0 |
| | | none | |
| Primary Th1 act | 0.0 | Lung Microvascular EC | 0.0 |
| | | TNF alpha + IL-1 beta | |
| Primary Th2 act | 0.0 | Microvascular Dermal EC | 0.0 |
| | | none | |
| Primary Tr1 act | 0.0 | Microsvasular Dermal EC | 0.0 |
| | | TNF alpha + IL-1 beta | |
| Primary Th1 rest | 0.0 | Bronchial epithelium | 0.0 |
| | | TNF alpha + IL1 beta | |
| Primary Th2 rest | 0.0 | Small airway epithelium | 0.0 |
| | | none | |
| Primary Tr1 rest | 0.0 | Small airway epithelium | 0.0 |
| | | TNF alpha + IL-1 beta | |
| CD45RA CD4 | 0.0 | Coronery artery SMC rest | 0.0 |
| lymphocyte act | | | |
| CD45RO CD4 | 0.0 | Coronery artery SMC | 0.0 |
| lymphocyte act | | TNF alpha + IL-1 beta | |
| CD8 lymphocyte act | 0.0 | Astrocytes rest | 0.0 |
| Secondary CD8 | 0.0 | Astrocytes TNF alpha + | 0.0 |
| lymphocyte rest | | IL-1 beta | |
| Secondary CD8 | 0.0 | KU-812 (Basophil) rest | 0.0 |
| lymphocyte act | | | |
| CD4 lymphocyte none | 0.0 | KU-812 (Basophil) | 0.0 |
| | | PMA/ionomycin | |
| 2ry Th1/Th2/Tr1_anti- | 0.0 | CCD1106 (Keratinocytes) | 0.0 |
| CD95 CH11 | | none | |
| LAK cells rest | 0.0 | CCD1106 (Keratinocytes) | 0.0 |
| | | TNF alpha + IL-1 beta | |
| LAK cells IL-2 | 0.0 | Liver cirrhosis | 1.7 |
| LAK cells IL-2 + IL-12 | 0.0 | Lupus kidney | 5.8 |
| LAK cells IL-2 + IFN | 0.0 | NCI-H292 none | 0.0 |
| gamma | | | |
| LAK cells IL-2 + IL-18 | 0.0 | NCI-H292 IL-4 | 0.0 |
| LAK cells | 0.0 | NCI-H292 IL-9 | 0.0 |
| PMA/ionomycin | | | |
| NK Cells IL-2 rest | 0.0 | NCI-H292 IL-13 | 0.0 |
| Two Way MLR 3 day | 0.0 | NCI-H292 IFN gamma | 0.0 |
| Two Way MLR 5 day | 0.0 | HPAEC none | 0.0 |
| Two Way MLR 7 day | 0.0 | HPAEC TNF alpha + IL-1 | 0.0 |
| | | beta | |
| PBMC rest | 0.0 | Lung fibroblast none | 0.0 |
| PBMC PWM | 0.0 | Lung fibroblast TNF alpha + | 0.0 |
| | | IL-1 beta | |
| PBMC PHA-L | 0.0 | Lung fibroblast IL-4 | 0.0 |
| Ramos (B cell) none | 0.0 | Lung fibroblast IL-9 | 0.0 |
| Ramos (B cell) | 0.0 | Lung fibroblast IL-13 | 0.0 |
| ionomycin | | | |
| B lymphocytes PWM | 0.0 | Lung fibroblast IFN | 0.0 |
| | | gamma | |
| B lymphocytes CD40L | 0.0 | Dermal fibroblast | 0.0 |
| and IL-4 | | CCD1070 rest | |
| EOL-1 dbcAMP | 0.0 | Dermal fibroblast | 0.0 |
| | | CCD1070 TNF alpha | |
| EOL-1 dbcAMP | 0.0 | Dermal fibroblast | 0.0 |
| PMA/ionomycin | | CCD1070 IL-1 beta | |
| Dendritic cells none | 0.0 | Dermal fibroblast IFN | 0.0 |
| | | gamma | |
| Dendritic cells LPS | 0.0 | Dermal fibroblast IL-4 | 0.0 |
| Dendritic cells anti- | 0.0 | IBD Colitis 2 | 0.0 |
| CD40 | | | |
| Monocytes rest | 0.0 | IBD Crohn's | 0.0 |
| Monocytes LPS | 0.0 | Colon | 2.3 |
| Macrophages rest | 0.0 | Lung | 0.0 |
| Macrophages LPS | 0.0 | Thymus | 100.0 |
| HUVEC none | 0.0 | Kidney | 7.6 |
| HUVEC starved | 0.0 | | |

General_screening_panel_v1.4 Summary: Ag2957 Expression of the NOV3b gene is restricted to placenta, fetal kidney and liver. Thus, expression of this gene could be used to differentiate between these samples and other samples on this panel. In addition, this gene shows no or very low expression in the cancer cell lines used in this panel. Thus, the absence of expression could potentially be used as a diagnostic marker for cancer.

Panel 1.3D Summary: Ag2957 Expression of the NOV3b gene is restricted to kidney, spinal cord and liver. Thus, expression of this gene could be used to differentiate between these samples and other samples on this panel. This gene encodes a putative claudin. Claudins are components of tight junction strands. Thus, this specific pattern of expression may indicate that this gene product is involved in the formation of TJ strands in these tissues.

Among the CNS regions on this panel, this tight junction protein is expressed only in the spinal cord and may be involved in the blood brain barrier in this region. This molecule may therefore be of utility in the treatment of spinal cord injury. Growth factors such as BDNF and NGF have been shown in animal models to enhance repair after spinal crush injury; however in the clinical condition it is hard to administer protein therapeutics due to the blood brain barrier. The selective downregulation of this molecule may therefore increase the amount of protein crossing the blood brain barrier in the spinal cord, while not hampering its function in the rest of the CNS.

In addition, this gene shows no or very low expression in the cancer cell lines used in this panel. Thus, the absence of expression could potentially be used as a diagnostic marker for cancer.

Panel 2D Summary: Ag2957 The NOV3b gene is consistently expressed in the normal kidney samples (CTs=32-33) but not in the adjacent kidney tumors. This result is in agreement with the expression in the previous panels. Thus, absence of expression of this gene could be used as a diagnostic marker for kidney cancer. Furthermore, therapeutic modulation of the function or expression of this gene may be a possible treatment for this cancer.

Panel 4D Summary: Ag2957 The expression of the NOV3b transcript is restricted to the thymus (CT=32.1) but not in T cells. Thus, expression of this transcript could be used as a marker for this tissue.

NOV1a, NOV1d, NOV1c, and NOV1b

Expression of gene NOV1a and variants NOV1d, NOV1c and NOV1b was assessed using the primer-probe sets Ag2954 and Ag2956, described in Tables DA and DB. Results of the RTQ-PCR runs are shown in Tables DC and DD.

TABLE DA
Probe Name Ag2954

| | | | Start | |
|---|---|---|---|---|
| Primers | Sequences | Length | Position | SEQ ID NO: |
| Forward | 5′-cttcagccactaccctcctt-3′ | 20 | 391 | 1009 |
| Probe | TET-5′-ccatgccacaatccaagacttctgg-3′-TAMRA | 25 | 428 | 1010 |
| Reverse | 5′-atgtcagggatgctgtcatc-3′ | 20 | 453 | 1011 |

TABLE DB
Probe Name Ag2956

| | | | Start | |
|---|---|---|---|---|
| Primers | Sequences | Length | Position | SEQ ID NO: |
| Forward | 5′-agtcagatggctccttcca-3′ | 19 | 643 | 1012 |
| Probe | TET-5′-cctcatgctaagacctaggaacctgg-3′-TAMRA | 26 | 662 | 1013 |
| Reverse | 5′-ccagagatccttggcagaag-3′ | 20 | 702 | 1014 |

TABLE DC
General_screening_panel_v1.4

| | Rel. Exp. (%) | Rel. Exp. (%) | | Rel. Exp. (%) | Rel. Exp. (%) |
|---|---|---|---|---|---|
| | Ag2954, Run | Ag2956, Run | | Ag2954, Run | Ag2956, Run |
| Tissue Name | 216607767 | 216607768 | Tissue Name | 216607767 | 216607768 |
| Adipose | 0.0 | 0.0 | Renal ca. TK-10 | 58.6 | 0.0 |
| Melanoma* | 0.0 | 0.0 | Bladder | 0.0 | 0.0 |
| Hs688(A).T | | | | | |
| Melanoma* | 0.0 | 0.0 | Gastric ca. (liver | 51.8 | 0.0 |
| Hs688(B).T | | | met.) NCI-N87 | | |
| Melanoma* | 25.0 | 0.0 | Gastric ca. | 0.0 | 0.0 |
| M14 | | | KATO III | | |
| Melanoma* | 0.0 | 0.0 | Colon ca. SW- | 0.0 | 0.0 |
| LOXIMVI | | | 948 | | |
| Melanoma* | 0.0 | 0.0 | Colon ca. SW480 | 20.9 | 0.0 |
| SK-MEL-5 | | | | | |
| Squamous cell | 0.0 | 0.0 | Colon ca.* | 0.0 | 0.0 |
| carcinoma | | | (SW480 met) | | |
| SCC-4 | | | SW620 | | |
| Testis Pool | 24.3 | 7.1 | Colon ca. HT29 | 0.0 | 0.0 |
| Prostate ca.* | 0.0 | 0.0 | Colon ca. HCT- | 0.0 | 0.0 |
| (bone met) | | | 116 | | |
| PC-3 | | | | | |
| Prostate Pool | 0.0 | 8.8 | Colon ca. CaCo-2 | 0.0 | 0.0 |
| Placenta | 0.0 | 0.0 | Colon cancer | 0.0 | 0.0 |
| | | | tissue | | |
| Uterus Pool | 0.0 | 0.0 | Colon ca. | 0.0 | 0.0 |
| | | | SW1116 | | |
| Ovarian ca. | 62.4 | 0.0 | Colon ca. Colo- | 0.0 | 0.0 |
| OVCAR-3 | | | 205 | | |
| Ovarian ca. | 0.0 | 7.9 | Colon ca. SW-48 | 0.0 | 0.0 |
| SK-OV-3 | | | | | |
| Ovarian ca. | 69.7 | 0.0 | Colon Pool | 99.3 | 0.0 |
| OVCAR-4 | | | | | |
| Ovarian ca. | 58.2 | 0.0 | Small Intestine | 0.0 | 10.1 |
| OVCAR-5 | | | Pool | | |
| Ovarian ca. | 18.7 | 0.0 | Stomach Pool | 25.9 | 10.3 |
| IGROV-1 | | | | | |
| Ovarian ca. | 0.0 | 0.0 | Bone Marrow | 28.3 | 0.0 |
| OVCAR-8 | | | Pool | | |
| Ovary | 0.0 | 0.0 | Fetal Heart | 0.0 | 0.0 |
| Breast ca. | 78.5 | 0.0 | Heart Pool | 0.0 | 0.0 |
| MCF-7 | | | | | |
| Breast ca. | 0.0 | 0.0 | Lymph Node | 0.0 | 0.0 |
| MDA-MB- | | | Pool | | |
| 231 | | | | | |
| Breast ca. BT | 0.0 | 0.0 | Fetal Skeletal | 22.5 | 1.7 |
| 549 | | | Muscle | | |
| Breast ca. | 25.5 | 0.0 | Skeletal Muscle | 62.0 | 26.2 |
| T47D | | | Pool | | |
| Breast ca. | 57.8 | 0.0 | Spleen Pool | 59.5 | 0.0 |
| MDA-N | | | | | |
| Breast Pool | 0.0 | 0.0 | Thymus Pool | 23.8 | 11.1 |
| Trachea | 0.0 | 0.0 | CNS cancer | 0.0 | 0.0 |
| | | | (glio/astro) U87- | | |
| | | | MG | | |
| Lung | 27.9 | 0.0 | CNS cancer | 0.0 | 9.7 |
| | | | (glio/astro) U- | | |
| | | | 118-MG | | |
| Fetal Lung | 0.0 | 0.0 | CNS cancer | 17.0 | 0.0 |
| | | | (neuro; met) SK- | | |
| | | | N-AS | | |
| Lung ca. NCI- | 0.0 | 0.0 | CNS cancer | 0.0 | 0.0 |
| N417 | | | (astro) SF-539 | | |
| Lung ca. LX-1 | 0.0 | 0.0 | CNS cancer | 0.0 | 0.0 |
| | | | (astro) SNB-75 | | |
| Lung ca. NCI- | 0.0 | 0.0 | CNS cancer | 0.0 | 0.0 |
| H146 | | | (glio) SNB-19 | | |
| Lung ca. | 0.0 | 0.0 | CNS cancer | 0.0 | 0.0 |
| SHP-77 | | | (glio) SF-295 | | |
| Lung ca. | 28.7 | 0.0 | Brain | 0.0 | 0.0 |
| A549 | | | (Amygdala) Pool | | |
| Lung ca. NCI- | 0.0 | 6.0 | Brain | 56.3 | 3.3 |
| H526 | | | (cerebellum) | | |
| Lung ca. NCI- | 28.1 | 11.0 | Brain (fetal) | 27.5 | 100.0 |
| H23 | | | | | |
| Lung ca. NCI- | 100.0 | 7.4 | Brain | 0.0 | 0.0 |
| H460 | | | (Hippocampus) | | |
| | | | Pool | | |
| Lung ca. | 0.0 | 0.0 | Cerebral Cortex | 0.0 | 0.0 |
| HOP-62 | | | Pool | | |
| Lung ca. NCI- | 0.0 | 0.0 | Brain (Substantia | 26.8 | 0.0 |
| H522 | | | nigra) Pool | | |
| Liver | 0.0 | 0.0 | Brain (Thalamus) | 0.0 | 0.0 |
| | | | Pool | | |
| Fetal Liver | 54.7 | 0.0 | Brain (whole) | 58.2 | 6.2 |
| Liver ca. | 0.0 | 0.0 | Spinal Cord Pool | 0.0 | 0.0 |
| HepG2 | | | | | |
| Kidney Pool | 0.0 | 8.0 | Adrenal Gland | 0.0 | 0.0 |
| Fetal Kidney | 0.0 | 0.0 | Pituitary gland | 25.3 | 5.4 |
| | | | Pool | | |
| Renal ca. 786-0 | 0.0 | 0.0 | Salivary Gland | 0.0 | 0.0 |
| Renal ca. | 0.0 | 0.0 | Thyroid (female) | 0.0 | 0.0 |
| A498 | | | | | |
| Renal ca. | 0.0 | 0.0 | Pancreatic ca. | 85.3 | 0.0 |
| ACHN | | | CAPAN2 | | |
| Renal ca. UO- | 0.0 | 0.0 | Pancreas Pool | 0.0 | 0.0 |
| 31 | | | | | |

TABLE DD
Panel 4D

| | Rel. | Rel. | | Rel. | Rel. |
|---|---|---|---|---|---|
| | Exp. (%) | Exp. (%) | | Exp. (%) | Exp. (%) |
| | Ag2954, | Ag2956, | | Ag2954, | Ag2956, |
| | Run | Run | | Run | Run |
| Tissue Name | 164329620 | 164401746 | Tissue Name | 164329620 | 164401746 |
| Secondary Th1 act | 0.0 | 0.0 | HUVEC IL-1beta | 48.3 | 0.0 |
| Secondary Th2 act | 0.0 | 0.0 | HUVEC IFN | 0.0 | 0.0 |
| | | | gamma | | |
| Secondary Tr1 act | 20.0 | 0.0 | HUVEC TNF | 0.0 | 0.0 |
| | | | alpha + IFN | | |
| | | | gamma | | |
| Secondary Th1 rest | 0.0 | 0.0 | HUVEC TNF | 0.0 | 0.0 |
| | | | alpha + IL4 | | |
| Secondary Th2 rest | 0.0 | 0.0 | HUVEC IL-11 | 0.0 | 0.0 |
| Secondary Tr1 rest | 0.0 | 0.0 | Lung | 0.0 | 0.0 |
| | | | Microvascular EC | | |
| | | | none | | |
| Primary Th1 act | 0.0 | 0.0 | Lung | 0.0 | 0.0 |
| | | | Microvascular EC | | |
| | | | TNF alpha + IL- | | |
| | | | 1beta | | |
| Primary Th2 act | 0.0 | 0.0 | Microvascular | 0.0 | 0.0 |
| | | | Dermal EC none | | |
| Primary Tr1 act | 0.0 | 0.0 | Microsvasular | 13.2 | 0.0 |
| | | | Dermal EC | | |
| | | | TNF alpha + IL- | | |
| | | | 1beta | | |
| Primary Th1 rest | 0.0 | 0.0 | Bronchial | 0.0 | 0.0 |
| | | | epithelium | | |
| | | | TNF alpha + | | |
| | | | IL1beta | | |
| Primary Th2 rest | 0.0 | 0.0 | Small airway | 38.7 | 0.0 |
| | | | epithelium none | | |
| Primary Tr1 rest | 17.0 | 0.0 | Small airway | 0.0 | 0.0 |
| | | | epithelium | | |
| | | | TNF alpha + IL- | | |
| | | | 1beta | | |
| CD45RA CD4 | 0.0 | 0.0 | Coronery artery | 0.0 | 19.1 |
| lymphocyte act | | | SMC rest | | |
| CD45RO CD4 | 0.0 | 0.0 | Coronery artery | 0.0 | 0.0 |
| lymphocyte act | | | SMC TNF alpha + | | |
| | | | IL-1beta | | |
| CD8 lymphocyte | 0.0 | 0.0 | Astrocytes rest | 0.0 | 0.0 |
| act | | | | | |
| Secondary CD8 | 0.0 | 0.0 | Astrocytes | 0.0 | 0.0 |
| lymphocyte rest | | | TNF alpha + IL- | | |
| | | | 1beta | | |
| Secondary CD8 | 0.0 | 0.0 | KU-812 | 0.0 | 0.0 |
| lymphocyte act | | | (Basophil) rest | | |
| CD4 lymphocyte | 0.0 | 0.0 | KU-812 | 0.0 | 0.0 |
| none | | | (Basophil) | | |
| | | | PMA/ionomycin | | |
| 2ry | 0.0 | 0.0 | CCD1106 | 0.0 | 0.0 |
| Th1/Th2/Tr1_anti- | | | (Keratinocytes) | | |
| CD95 CH11 | | | none | | |
| LAK cells rest | 0.0 | 0.0 | CCD1106 | 0.0 | 0.0 |
| | | | (Keratinocytes) | | |
| | | | TNF alpha + IL- | | |
| | | | 1beta | | |
| LAK cells IL-2 | 0.0 | 0.0 | Liver cirrhosis | 72.2 | 100.0 |
| LAK cells IL-2 + IL- | 0.0 | 0.0 | Lupus kidney | 0.0 | 0.0 |
| 12 | | | | | |
| LAK cells IL- | 11.3 | 0.0 | NCI-H292 none | 0.0 | 0.0 |
| 2 + IFN gamma | | | | | |
| LAK cells IL-2 + | 0.0 | 0.0 | NCI-H292 IL-4 | 14.5 | 0.0 |
| IL-18 | | | | | |
| LAK cells | 0.0 | 0.0 | NCI-H292 IL-9 | 0.0 | 0.0 |
| PMA/ionomycin | | | | | |
| NK Cells IL-2 rest | 0.0 | 0.0 | NCI-H292 IL-13 | 0.0 | 0.0 |
| Two Way MLR 3 | 0.0 | 0.0 | NCI-H292 IFN | 0.0 | 0.0 |
| day | | | gamma | | |
| Two Way MLR 5 | 0.0 | 0.0 | HPAEC none | 0.0 | 0.0 |
| day | | | | | |
| Two Way MLR 7 | 24.7 | 0.0 | HPAEC TNF | 0.0 | 0.0 |
| day | | | alpha + IL-1beta | | |
| PBMC rest | 6.9 | 0.0 | Lung fibroblast | 100.0 | 0.0 |
| | | | none | | |
| PBMC PWM | 15.3 | 0.0 | Lung fibroblast | 0.0 | 0.0 |
| | | | TNF alpha + IL-1 | | |
| | | | beta | | |
| PBMC PHA-L | 0.0 | 0.0 | Lung fibroblast | 0.0 | 0.0 |
| | | | IL-4 | | |
| Ramos (B cell) | 0.0 | 0.0 | Lung fibroblast | 9.7 | 32.8 |
| none | | | IL-9 | | |
| Ramos (B cell) | 10.7 | 0.0 | Lung fibroblast | 0.0 | 0.0 |
| ionomycin | | | IL-13 | | |
| B lymphocytes | 13.0 | 0.0 | Lung fibroblast | 0.0 | 0.0 |
| PWM | | | IFN gamma | | |
| B lymphocytes | 0.0 | 0.0 | Dermal fibroblast | 0.0 | 0.0 |
| CD40L and IL-4 | | | CCD1070 rest | | |
| EOL-1 dbcAMP | 0.0 | 0.0 | Dermal fibroblast | 13.1 | 0.0 |
| | | | CCD1070 TNF | | |
| | | | alpha | | |
| EOL-1 dbcAMP | 0.0 | 0.0 | Dermal fibroblast | 0.0 | 0.0 |
| PMA/ionomycin | | | CCD1070 IL-1 | | |
| | | | beta | | |
| Dendritic cells | 0.0 | 0.0 | Dermal fibroblast | 0.0 | 0.0 |
| none | | | IFN gamma | | |
| Dendritic cells LPS | 0.0 | 0.0 | Dermal fibroblast | 34.2 | 26.8 |
| | | | IL-4 | | |
| Dendritic cells anti- | 0.0 | 0.0 | IBD Colitis 2 | 34.9 | 0.0 |
| CD40 | | | | | |
| Monocytes rest | 0.0 | 0.0 | IBD Crohn's | 20.0 | 0.0 |
| Monocytes LPS | 0.0 | 0.0 | Colon | 0.0 | 0.0 |
| Macrophages rest | 13.0 | 0.0 | Lung | 0.0 | 0.0 |
| Macrophages LPS | 24.7 | 0.0 | Thymus | 12.2 | 0.0 |
| HUVEC none | 0.0 | 0.0 | Kidney | 0.0 | 0.0 |
| HUVEC starved | 40.1 | 0.0 | | | |

CNS_neurodegeneration_v1.0 Summary: Ag2954/Ag2956 Expression of the NOV11 gene is low/undetectable in all samples on this panel (CTs>35). (Data not shown.)

General_screening_panel_v1.4 Summary: Ag2954 The NOV1a gene is expressed at a very low level or not at all in most of the cancer cell lines on this panel. Very low expression in cell lines from pancreatic, lung, breast and ovarian cancers suggests that it may be involved in these cancers.

Ag2956 This gene is a member of the claudin family of proteins, and is only expressed in the fetal brain. It may be involved in the process of axonal growth or targeting and synaptogenesis (specifically in the development of tight junctions between neurons and other cell types). Therefore, this gene product may be of therapeutic benefit in the treatment of neuronal loss in clinical conditions such as head trauma or stroke where increased compensatory synaptogenesis is desireable.

Panel 1.3D Summary: Ag2954/Ag2956 Expression of the NOV11 gene is low/undetectable in all samples on this panel (CTs>35). (Data not shown.)

Panel 4D Summary: Ag2954/Ag2956 Two experiments with two different sets of primers show low but significant levels of expression of this transcript in liver cirrhosis, dermal and lung fibroblasts and endothelium. Thus, the NOV11 transcript may serve as a marker for these tissues and play a role in maintaining the integrity of these tissues.

NOV2

Expression of gene NOV2 was assessed using the primer-probe set Ag2958, described in Table EA. Results of the RTQ-PCR runs are shown in Table EB.

TABLE EA
Probe Name Ag2958

| | | | Start | |
|---|---|---|---|---|
| Primers | Sequences | Length | Position | SEQ ID NO: |
| Forward | 5′-gttgtcagggtagagcagaaga-3′ | 22 | 341 | 1015 |
| Probe | TET-5′-ccaccaagaaaccttttgcaataaaa-3′-TAMRA | 26 | 363 | 1016 |
| Reverse | 5′-taccttccctctctctggtttc-3′ | 22 | 395 | 1017 |

TABLE EB
Panel 1.3D

| | Rel. Exp. (%) Ag2958, | | Rel. Exp. (%) Ag2958, |
|---|---|---|---|
| Tissue Name | Run 167809085 | Tissue Name | Run 167809085 |
| Liver adenocarcinoma | 0.0 | Kidney (fetal) | 0.4 |
| Pancreas | 0.0 | Renal ca. 786-0 | 0.0 |
| Pancreatic ca. CAPAN2 | 0.0 | Renal ca. A498 | 0.0 |
| Adrenal gland | 0.0 | Renal ca. RXF 393 | 0.0 |
| Thyroid | 0.0 | Renal ca. ACHN | 0.0 |
| Salivary gland | 0.0 | Renal ca. UO-31 | 0.0 |
| Pituitary gland | 0.0 | Renal ca. TK-10 | 0.0 |
| Brain (fetal) | 0.0 | Liver | 0.0 |
| Brain (whole) | 0.0 | Liver (fetal) | 0.0 |
| Brain (amygdala) | 0.0 | Liver ca. | 0.0 |
| | | (hepatoblast) HepG2 | |
| Brain (cerebellum) | 0.0 | Lung | 0.0 |
| Brain (hippocampus) | 0.0 | Lung (fetal) | 0.0 |
| Brain (substantia nigra) | 0.0 | Lung ca. (small cell) | 0.0 |
| | | LX-1 | |
| Brain (thalamus) | 0.0 | Lung ca. (small cell) | 0.0 |
| | | NCI-H69 | |
| Cerebral Cortex | 0.0 | Lung ca. (s.cell var.) | 0.0 |
| | | SHP-77 | |
| Spinal cord | 0.6 | Lung ca. (large | 0.0 |
| | | cell)NCI-H460 | |
| glio/astro U87-MG | 0.0 | Lung ca. (non-sm. | 0.0 |
| | | cell) A549 | |
| glio/astro U-118-MG | 0.0 | Lung ca. (non-s.cell) | 0.0 |
| | | NCI-H23 | |
| astrocytoma SW1783 | 0.0 | Lung ca. (non-s.cell) | 0.0 |
| | | HOP-62 | |
| neuro*; met SK-N-AS | 0.0 | Lung ca. (non-s.cl) | 0.0 |
| | | NCI-H522 | |
| astrocytoma SF-539 | 0.0 | Lung ca. (squam.) | 0.0 |
| | | SW 900 | |
| astrocytoma SNB-75 | 0.0 | Lung ca. (squam.) | 0.0 |
| | | NCI-H596 | |
| glioma SNB-19 | 0.0 | Mammary gland | 0.0 |
| glioma U251 | 0.0 | Breast ca.* (pl.ef) | 0.4 |
| | | MCF-7 | |
| glioma SF-295 | 0.0 | Breast ca.* (pl.ef) | 0.0 |
| | | MDA-MB-231 | |
| Heart (fetal) | 0.0 | Breast ca.* (pl.ef) | 0.0 |
| | | T47D | |
| Heart | 0.0 | Breast ca. BT-549 | 0.0 |
| Skeletal muscle (fetal) | 0.0 | Breast ca. MDA-N | 0.0 |
| Skeletal muscle | 0.0 | Ovary | 0.0 |
| Bone marrow | 0.0 | Ovarian ca. OVCAR-3 | 2.0 |
| Thymus | 0.0 | Ovarian ca. OVCAR-4 | 0.0 |
| Spleen | 0.0 | Ovarian ca. OVCAR-5 | 0.0 |
| Lymph node | 0.0 | Ovarian ca. OVCAR-8 | 0.0 |
| Colorectal | 0.0 | Ovarian ca. IGROV-1 | 0.0 |
| Stomach | 0.0 | Ovarian ca.* (ascites) | 0.0 |
| | | SK-OV-03 | |
| Small intestine | 0.0 | Uterus | 0.0 |
| Colon ca. SW480 | 0.0 | Placenta | 0.0 |
| Colon ca.* | 0.0 | Prostate | 0.0 |
| SW620(SW480 met) | | | |
| Colon ca. HT29 | 0.0 | Prostate ca.* (bone | 0.0 |
| | | met)PC-3 | |
| Colon ca. HCT-116 | 0.0 | Testis | 0.0 |
| Colon ca. CaCo-2 | 0.0 | Melanoma | 0.0 |
| | | Hs688(A).T | |
| Colon ca. | 0.0 | Melanoma* (met) | 0.0 |
| tissue(ODO3866) | | Hs688(B).T | |
| Colon ca. HCC-2998 | 0.0 | Melanoma UACC-62 | 0.0 |
| Gastric ca.* (liver met) | 0.0 | Melanoma M14 | 0.0 |
| NCI-N87 | | | |
| Bladder | 0.0 | Melanoma LOX | 0.0 |
| | | IMVI | |
| Trachea | 0.0 | Melanoma* (met) | 0.0 |
| | | SK-MEL-5 | |
| Kidney | 100.0 | Adipose | 0.0 |

Panel 1.3D Summary: Ag2958 Expression of the NOV2 is restricted to the kidney (CT=31.8). In addition, this gene is expressed at higher levels in adult kidney when compared to expression in fetal kidney (CT value=40). Thus, this gene product may be useful for the differentiation of adult and fetal kidney tissue. This highly specific expression pattern also suggests that this gene product may be a small molecule drug for the treatment of diseases of the kidney.

Panel 4D Summary: Ag2958 Expression of the NOV2 gene is low/undetectable in all samples on this panel (CTs>35). (Data not shown.)

NOV10

Expression of gene NOV10, also known as CG55964-02, was assessed using the primer-probe set Ag2857, described in Table FA.

TABLE FA
Probe Name Ag2857

| | | | Start | |
|---|---|---|---|---|
| Primers | Sequences | Length | Position | SEQ ID NO: |
| Forward | 5′-catttgccagagatttcttttg-3′ | 22 | 300 | 1018 |
| Probe | TET-5′-caaatgtggcttattcactcattccagg-3′-TAMRA | 28 | 336 | 1019 |
| Reverse | 5′-agaaggatacccgattcaattg-3′ | 22 | 364 | 1020 |

CNS_neurodegeneration_v1.0 Summary: Ag2857 Expression of the NOV10 gene is low/undetectable in all samples on this panel. (CTs>35). (Data not shown.)

Panel 1.3D Summary: Ag2857 Expression of the NOV10 gene is low/undetectable in all samples on this panel. (CTs>35). (Data not shown.)

Panel 2.2 Summary: Ag2857 Expression of the NOV10 gene is low/undetectable in all samples on this panel. (CTs>35). (Data not shown.)

Panel 4D Summary: Ag2857 Expression of the NOV10 gene is low/undetectable in all samples on this panel. (CTs>35). (Data not shown.)

NOV11

Expression of gene NOV11 was assessed using the primer-probe set Ag2858, described in Table GA.

TABLE GA
Probe Name Ag2858

| | | | Start | |
|---|---|---|---|---|
| Primers | Sequence | Length | Position | SEQ ID NO: |
| Forward | 5′-cttaagtcagtcctggcagttg-3′ | 22 | 674 | 1021 |
| Probe | TET-5′-aaattatttcagacctgcatctccca-3′-TAMRA | 26 | 716 | 1022 |
| Reverse | 5′-agaacacaaggacagcacagat-3′ | 22 | 743 | 1023 |

CNS_neurodegeneration_v1.0 Summary: Ag2858 Expressin of the NOV11 gene is low/undetectable in all samples on this panel (CTs>35). (Data not shown.) The amp plot indicates that there is a high probability of a probe failure.

Panel 1.3D Summary: Ag2858 Expression of the NOV11 gene is low/undetectable in all samples on this panel (CTs>35). (Data not shown.) The amp plot indicates that there is a high probability of a probe failure.

Panel 2.2 Summary: Ag2858 Expression of the NOV11 gene is low/undetectable in all samples on this panel (CTs>35). (Data not shown.) The amp plot indicates that there is a high probability of a probe failure.

Panel 4D Summary: Ag2858 Expression of the NOV11 gene is low/undetectable in all samples on this panel (CTs>35). (Data not shown.) The amp plot indicates that there is a high probability of a probe failure.

NOV12

Expression of gene NOV12 was assessed using the primer-probe set Ag2867, described in Table HA. Results of the RTQ-PCR runs are shown in Tables HB and HC.

TABLE HA
Probe Name Ag2867

| | | | Start | |
|---|---|---|---|---|
| Primers | Sequence | Length | Position | SEQ ID NO: |
| Forward | 5′-gtcacaccttgtgactgcaa-3′ | 20 | 176 | 1024 |
| Probe | TET-5′-acttacttgtgtgcagtgccatgcct-3′-TAMRA | 26 | 196 | 1025 |
| Reverse | 5′-cattggaaacctttcaggaaa-3′ | 21 | 236 | 1026 |

TABLE HB
CNS_neurodegeneration_v1.0

| | Rel. Exp. (%) Ag2867, | | Rel. Exp. (%) Ag2867, |
|---|---|---|---|
| Tissue Name | 208699903 | Tissue Name | Run 208699903 |
| AD 1 Hippo | 7.9 | Control (Path) 3 | 1.5 |
| | | Temporal Ctx | |
| AD 2 Hippo | 20.3 | Control (Path) 4 | 22.1 |
| | | Temporal Ctx | |
| AD 3 Hippo | 5.0 | AD 1 Occipital Ctx | 13.4 |
| AD 4 Hippo | 6.6 | AD 2 Occipital Ctx | 0.0 |
| | | (Missing) | |
| AD 5 hippo | 65.5 | AD 3 Occipital Ctx | 3.6 |
| AD 6 Hippo | 44.4 | AD 4 Occipital Ctx | 12.9 |
| Control 2 Hippo | 14.3 | AD 5 Occipital Ctx | 15.4 |
| Control 4 Hippo | 9.1 | AD 6 Occipital Ctx | 8.7 |
| Control (Path) 3 | 5.6 | Control 1 Occipital | 0.7 |
| Hippo | | Ctx | |
| AD 1 Temporal Ctx | 100.0 | Control 2 Occipital | 1.4 |
| | | Ctx | |
| AD 2 Temporal Ctx | 12.6 | Control 3 Occipital | 14.5 |
| | | Ctx | |
| AD 3 Temporal Ctx | 7.6 | Control 4 Occipital | 3.4 |
| | | Ctx | |
| AD 4 Temporal Ctx | 14.0 | Control (Path) 1 | 62.0 |
| | | Occipital Ctx | |
| AD 5 Inf Temporal | 55.9 | Control (Path) 2 | 8.1 |
| Ctx | | Occipital Ctx | |
| AD 5 Sup Temporal | 71.2 | Control (Path) 3 | 0.0 |
| Ctx | | Occipital Ctx | |
| AD 6 Inf Temporal | 45.7 | Control (Path) 4 | 21.0 |
| Ctx | | Occipital Ctx | |
| AD 6 Sup Temporal | 16.5 | Control 1 Parietal | 0.9 |
| Ctx | | Ctx | |
| Control 1 Temporal | 0.9 | Control 2 Parietal | 8.3 |
| Ctx | | Ctx | |
| Control 2 Temporal | 5.4 | Control 3 Parietal | 4.4 |
| Ctx | | Ctx | |
| Control 3 Temporal | 2.5 | Control (Path) 1 | 40.1 |
| Ctx | | Parietal Ctx | |
| Control 4 Temporal | 6.3 | Control (Path) 2 | 20.7 |
| Ctx | | Parietal Ctx | |
| Control (Path) 1 | 39.8 | Control (Path) 3 | 5.2 |
| Temporal Ctx | | Parietal Ctx | |
| Control (Path) 2 | 24.8 | Control (Path) 4 | 21.3 |
| Temporal Ctx | | Parietal Ctx | |

TABLE HC
Panel 4D

| | Rel. | | Rel. |
|---|---|---|---|
| | Exp. (%) Ag2867, | | Exp. (%) Ag2867, |
| Tissue Name | Run 164311002 | Tissue Name | Run 164311002 |
| Secondary Th1 | 5.6 | HUVEC IL-1 beta | 4.2 |
| act | | | |
| Secondary Th2 | 15.7 | HUVEC IFN | 1.9 |
| act | | gamma | |
| Secondary Tr1 | 11.8 | HUVEC TNF alpha + | 2.7 |
| act | | IFN gamma | |
| Secondary Th1 | 3.9 | HUVEC TNF alpha + | 2.9 |
| rest | | IL4 | |
| Secondary Th2 | 17.3 | HUVEC IL-11 | 2.3 |
| rest | | | |
| Secondary Tr1 | 12.2 | Lung Microvascular | 6.1 |
| rest | | EC none | |
| Primary Th1 act | 38.4 | Lung Microvascular | 7.2 |
| | | EC TNF alpha + IL-1 beta | |
| Primary Th2 act | 22.2 | Microvascular | 6.7 |
| | | Dermal EC none | |
| Primary Tr1 act | 29.7 | Microsvasular | 3.9 |
| | | Dermal EC TNF alpha + IL- | |
| | | 1 beta | |
| Primary Th1 rest | 93.3 | Bronchial epithelium | 9.0 |
| | | TNF alpha + IL1 beta | |
| Primary Th2 rest | 55.9 | Small airway | 1.7 |
| | | epithelium none | |
| Primary Tr1 rest | 39.2 | Small airway | 16.7 |
| | | epithelium TNF alpha + IL- | |
| | | 1 beta | |
| CD45RA CD4 | 6.0 | Coronery artery | 3.2 |
| lymphocyte act | | SMC rest | |
| CD45RO CD4 | 15.6 | Coronery artery | 1.3 |
| lymphocyte act | | SMC TNF alpha + IL-1 beta | |
| CD8 lymphocyte | 16.0 | Astrocytes rest | 8.8 |
| act | | | |
| Secondary CD8 | 17.2 | Astrocytes | 6.2 |
| lymphocyte rest | | TNF alpha + IL-1 beta | |
| Secondary CD8 | 13.6 | KU-812 (Basophil) | 0.7 |
| lymphocyte act | | rest | |
| CD4 lymphocyte | 22.4 | KU-812 (Basophil) | 0.0 |
| none | | PMA/ionomycin | |
| 2ry | 24.0 | CCD1106 | 7.0 |
| Th1/Th2/Tr1_anti-CD95 | | (Keratinocytes) none | |
| CH11 | | | |
| LAK cells rest | 23.7 | CCD1106 | 2.4 |
| | | (Keratinocytes) TNF alpha + | |
| | | IL-1 beta | |
| LAK cells IL-2 | 29.9 | Liver cirrhosis | 3.6 |
| LAK cells IL- | 32.8 | Lupus kidney | 1.1 |
| 2 + IL-12 | | | |
| LAK cells IL- | 50.7 | NCI-H292 none | 17.8 |
| 2 + IFN gamma | | | |
| LAK cells IL-2 + | 39.0 | NCI-H292 IL-4 | 15.5 |
| IL-18 | | | |
| LAK cells | 4.1 | NCI-H292 IL-9 | 13.9 |
| PMA/ionomycin | | | |
| NK Cells IL-2 | 22.5 | NCI-H292 IL-13 | 7.2 |
| rest | | | |
| Two Way MLR | 26.1 | NCI-H292 IFN | 8.2 |
| 3 day | | gamma | |
| Two Way MLR | 9.3 | HPAEC none | 2.7 |
| 5 day | | | |
| Two Way MLR | 12.0 | HPAEC TNF alpha + | 2.7 |
| 7 day | | IL-1 beta | |
| PBMC rest | 11.6 | Lung fibroblast none | 5.5 |
| PBMC PWM | 65.1 | Lung fibroblast TNF | 2.8 |
| | | alpha + IL-1 beta | |
| PBMC PHA-L | 17.1 | Lung fibroblast IL-4 | 7.2 |
| Ramos (B cell) | 13.9 | Lung fibroblast IL-9 | 6.9 |
| none | | | |
| Ramos (B cell) | 23.8 | Lung fibroblast IL- | 6.5 |
| ionomycin | | 13 | |
| B lymphocytes | 73.2 | Lung fibroblast IFN | 4.6 |
| PWM | | gamma | |
| B lymphocytes | 100.0 | Dermal fibroblast | 12.7 |
| CD40L and IL-4 | | CCD1070 rest | |
| EOL-1 dbcAMP | 3.1 | Dermal fibroblast | 40.1 |
| | | CCD1070 TNF alpha | |
| EOL-1 dbcAMP | 5.2 | Dermal fibroblast | 6.0 |
| PMA/ionomycin | | CCD1070 IL-1 beta | |
| Dendritic cells | 6.5 | Dermal fibroblast | 2.6 |
| none | | IFN gamma | |
| Dendritic cells | 3.1 | Dermal fibroblast | 5.8 |
| LPS | | IL-4 | |
| Dendritic cells | 4.2 | IBD Colitis 2 | 2.7 |
| anti-CD40 | | | |
| Monocytes rest | 8.5 | IBD Crohn's | 5.5 |
| Monocytes LPS | 5.0 | Colon | 22.5 |
| Macrophages rest | 4.2 | Lung | 6.7 |
| Macrophages | 2.4 | Thymus | 6.7 |
| LPS | | | |
| HUVEC none | 2.6 | Kidney | 92.7 |
| HUVEC starved | 12.7 | | |

CNS_neurodegeneration_v1.0 Summary: Ag2867 The NOV12 gene represents a novel G-protein coupled receptor (GPCR) with expression in the brain. The GPCR family of receptors contains a large number of neurotransmitter receptors, including the dopamine, serotonin, a and b-adrenergic, acetylcholine muscarinic, histamine, peptide, and metabotropic glutamate receptors. GPCRs are excellent drug targets in various neurologic and psychiatric diseases. All antipsychotics have been shown to act at the dopamine D2 receptor; similarly novel antipsychotics also act at the serotonergic receptor, and often the muscarinic and adrenergic receptors as well. While the majority of antidepressants can be classified as selective serotonin reuptake inhibitors, blockade of the 5-HT1A and a2 adrenergic receptors increases the effects of these drugs. The GPCRs are also of use as drug targets in the treatment of stroke. Blockade of the glutamate receptors may decrease the neuronal death resulting from excitotoxicity; further more the purinergic receptors have also been implicated as drug targets in the treatment of cerebral ischemia. The b-adrenergic receptors have been implicated in the treatment of ADHD with Ritalin, while the a-adrenergic receptors have been implicated in memory. Therefore this gene may be of use as a small molecule target for the treatment of any of the described diseases.

In addition, this GPCR is found to be upregulated in the temporal cortex of Alzheimer's disease patients. Blockade of this receptor may be of use in the treatment of this disease and decrease neuronal death.

References:

El Yacoubi M, Ledent C, Parmentier M, Bertorelli R, Ongini E, Costentin J, Vaugeois J M. Adenosine A2A receptor antagonists are potential antidepressants: evidence based on pharmacology and A2A receptor knockout mice. Br J Pharmacol September 2001: 134(1):68-77

1. Adenosine, an ubiquitous neuromodulator, and its analogues have been shown to produce ‘depressant’ effects in animal models believed to be relevant to depressive disorders, while adenosine receptor antagonists have been found to reverse adenosine-mediated ‘depressant’ effect. 2. We have designed studies to assess whether adenosine A2A receptor antagonists, or genetic inactivation of the receptor would be effective in established screening procedures, such as tail suspension and forced swim tests, which are predictive of clinical antidepressant activity. 3. Adenosine A2A receptor knockout mice were found to be less sensitive to ‘depressant’ challenges than their wildtype littermates. Consistently, the adenosine A2A receptor blockers SCH 58261 (1-10 mg kg(−1), i.p.) and KW 6002 (0.1-10 mg kg(−1), p.o.) reduced the total immobility time in the tail suspension test. 4. The efficacy of adenosine A2A receptor antagonists in reducing immobility time in the tail suspension test was confirmed and extended in two groups of mice. Specifically, SCH 58261 (1-10 mg kg(−1)) and ZM 241385 (15-60 mg kg(−1)) were effective in mice previously screened for having high immobility time, while SCH 58261 at 10 mg kg(−1) reduced immobility of mice that were selectively bred for their spontaneous ‘helplessness’ in this assay. 5. Additional experiments were carried out using the forced swim test. SCH 58261 at 10 mg kg(−1) reduced the immobility time by 61%, while KW 6002 decreased the total immobility time at the doses of 1 and 10 mg kg(−1) by 75 and 79%, respectively. 6. Administration of the dopamine D2 receptor antagonist haloperidol (50-200 microg kg(−1) i.p.) prevented the antidepressant-like effects elicited by SCH 58261 (10 mg kg(−1) i.p.) in forced swim test whereas it left unaltered its stimulant motor effects. 7. In conclusion, these data support the hypothesis that A2A receptor antagonists prolong escape-directed behaviour in two screening tests for antidepressants. Altogether the results support the hypothesis that blockade of the adenosine A2A receptor might be an interesting target for the development of effective antidepressant agents.

Blier P. Pharmacology of rapid-onset antidepressant treatment strategies. Clin Psychiatry 2001;62 Suppl 15:12-7

Although selective serotonin reuptake inhibitors (SSRIs) block serotonin (5-HT) reuptake rapidly, their therapeutic action is delayed. The increase in synaptic 5-HT activates feedback mechanisms mediated by 5-HT1A (cell body) and 5-HT1B (terminal) autoreceptors, which, respectively, reduce the firing in 5-HT neurons and decrease the amount of 5-HT released per action potential resulting in attenuated 5-HT neurotransmission. Long-term treatment desensitizes the inhibitory 5-HT1 autoreceptors, and 5-HT neurotransmission is enhanced. The time course of these events is similar to the delay of clinical action. The addition of pindolol, which blocks 5-HT1A receptors, to SSRI treatment decouples the feedback inhibition of 5-HT neuron firing and accelerates and enhances the antidepressant response. The neuronal circuitry of the 5-HT and norepinephrine (NE) systems and their connections to forebrain areas believed to be involved in depression has been dissected. The firing of 5-HT neurons in the raphe nuclei is driven, at least partly, by alpha1-adrenoceptor-mediated excitatory inputs from NE neurons. Inhibitory alpha2-adrenoceptors on the NE neuroterminals form part of a feedback control mechanism. Mirtazapine, an antagonist at alpha2-adrenoceptors, does not enhance 5-HT neurotransmission directly but disinhibits the NE activation of 5-HT neurons and thereby increases 5-HT neurotransmission by a mechanism that does not require a time-dependent desensitization of receptors. These neurobiological phenomena may underlie the apparently faster onset of action of mirtazapine compared with the SSRIs.

Tranquillini M E, Reggiani A. Glycine-site antagonists and stroke. Expert Opin Investig Drugs November 1999;8(11):1837-1848

The excitatory amino acid, (S)-glutamic acid, plays an important role in controlling many neuronal processes. Its action is mediated by two main groups of receptors: the ionotropic receptors (which include NMDA, AMPA and kainic acid subtypes) and the metabotropic receptors (mGluR(1-8)) mediating G-protein coupled responses. This review focuses on the strychnine insensitive glycine binding site located on the NMDA receptor channel, and on the possible use of selective antagonists for the treatment of stroke. Stroke is a devastating disease caused by a sudden vascular accident. Neurochemically, a massive release of glutamate occurs in neuronal tissue; this overactivates the NMDA receptor, leading to increased intracellular calcium influx, which causes neuronal cell death through necrosis. NMDA receptor activation strongly depends upon the presence of glycine as a co-agonist. Therefore, the administration of a glycine antagonist can block overactivation of NMDA receptors, thus preserving neurones from damage. The glycine antagonists currently identified can be divided into five main categories depending on their chemical structure: indoles, tetrahydroquinolines, benzoazepines, quinoxalinediones and pyrida-zinoquinolines.

Monopoli A, Lozza G, Forlani A, Mattavelli A, Ongini E. Blockade of adenosine A2A receptors by SCH 58261 results in neuroprotective effects in cerebral ischaemia in rats. Neuroreport December 1, 1998;9(17):3955-9

Blockade of adenosine receptors can reduce cerebral infarct size in the model of global ischaemia. Using the potent and selective A2A adenosine receptor antagonist, SCH 58261, we assessed whether A2A receptors are involved in the neuronal damage following focal cerebral ischaemia as induced by occluding the left middle cerebral artery. SCH 58261 (0.01 mg/kg either i.p. or i.v.) administered to normotensive rats 10 min after ischaemia markedly reduced cortical infarct volume as measured 24 h later (30% vs controls, p<0.05). Similar effects were observed when SCH 58261 (0.01 mg/kg, i.p.) was administered to hypertensive rats (28% infarct volume reduction vs controls, p<0.05). Neuroprotective properties of SCH 58261 administered after ischaemia indicate that blockade of A2A adenosine receptors is a potentially useful biological target for the reduction of brain injury.

Panel 1.3D Summary: Ag2867 Results from one experiment with the NOV12 gene are not included. The amp plot indicates that there were experimental difficulties with this run.

Panel 2.2 Summary: Ag2867 Results from one experiment with the NOV12 gene are not included. The amp plot indicates that there were experimental difficulties with this run.

Panel 4D Summary: Ag2867 Expression of the NOV12 gene is widespread among samples in this panel, with highest expression in B lymphocytes stimulated with CD40L and IL-4 (CT=31.1).

This transcript is also highly expressed in activated B cells and primary resting Th1 and Th2 T cells. The expression of this transcript in PBMC treated with the B cell mitogen, PWM, confirms the importance of CG54575-01 gene expression in activated B cells. In addition, this transcript is also abundantly expressed on primary resting Th1 cells (to a lesser degree on primary resting Th2 cells). Therefore, it appears that this gene, encoding a GPCR homolog, is a potential new member of the chemokine receptor family. The expression of this protein in activated B cells suggests a role for this protein in their trafficking to appropriate sites where they can fully activate antigen specific T cells. Thus, the protein encoded by this gene is likely to participate in the development of immune or inflammatory reactions.

In addition, the high expression of this gene in the kidney suggests that the putative GPCR encoded for by this gene could allow cells within the kidney to respond to specific microenvironmental signals (For example, ref. 1). Therefore, antibody or small molecule therapies designed with the protein encoded for by this gene could modulate kidney function and be important in the treatment of inflammatory or autoimmune diseases that affect the kidney, including lupus and glomerulonephritis.

References:

References:

Mark M. D., Wittemann S., Herlitze S. (2000) G protein modulation of recombinant P/Q-type calcium channels by regulators of G protein signalling proteins. J. Physiol. 528 Pt 1: 65-77.

1. Fast synaptic transmission is triggered by the activation of presynaptic Ca2+ channels which can be inhibited by Gbetagamma subunits via G protein-coupled receptors (GPCR). Regulators of G protein signalling (RGS) proteins are GTPase-accelerating proteins (GAPs), which are responsible for >100-fold increases in the GTPase activity of G proteins and might be involved in the regulation of presynaptic Ca2+ channels. In this study we investigated the effects of RGS2 on G protein modulation of recombinant P/Q-type channels expressed in a human embryonic kidney (HEK293) cell line using whole-cell recordings. 2. RGS2 markedly accelerates transmitter-mediated inhibition and recovery from inhibition of Ba2+ currents (IBa) through P/Q-type channels heterologously expressed with the muscarinic acetylcholine receptor M2 (mAChR M2). 3. Both RGS2 and RGS4 modulate the prepulse facilitation properties of P/Q-type Ca2+ channels. G protein reinhibition is accelerated, while release from inhibition is slowed. These kinetics depend on the availability of G protein alpha and betagamma subunits which is altered by RGS proteins. 4. RGS proteins unmask the Ca2+ channel beta subunit modulation of Ca2+ channel G protein inhibition. In the presence of RGS2, P/Q-type channels containing the beta2a and beta3 subunits reveal significantly altered kinetics of G protein modulation and increased facilitation compared to Ca2+ channels coexpressed with the beta1b or beta4 subunit.

PMID: 11018106

NOV13a and NOV13b

Expression of gene NOV13a and variant NOV13b was assessed using the primer-probe set Ag2869, described in Table IA. Results of the RTQ-PCR runs are shown in Table IB.

TABLE IA
Probe Name Ag2869

| | | | Start | |
|---|---|---|---|---|
| Primers | Sequence | Length | Position | SEQ ID NO: |
| Forward | 5′-tgtctgtggtagacaccacctt-3′ | 22 | 475 | 1027 |
| Probe | TET-5′-ctgaggctaccctaccgaggcagtaa-3′-TAMRA | 26 | 501 | 1028 |
| Reverse | 5′-cacaaaagaaatgagcaatgct-3′ | 22 | 528 | 1029 |

TABLE IB
Panel 4D

| | Rel. Exp. (%) | | Rel. Exp. (%) |
|---|---|---|---|
| | Ag2869, Run | | Ag2869, Run |
| Tissue Name | 164311007 | Tissue Name | 164311007 |
| Secondary Th1 act | 5.5 | HUVEC IL-1 beta | 1.7 |
| Secondary Th2 act | 68.3 | HUVEC IFN gamma | 9.5 |
| Secondary Tr1 act | 33.9 | HUVEC TNF alpha + IFN | 5.6 |
| | | gamma | |
| Secondary Th1 rest | 0.0 | HUVEC TNF alpha + IL4 | 8.9 |
| Secondary Th2 rest | 7.3 | HUVEC IL-11 | 5.5 |
| Secondary Tr1 rest | 3.0 | Lung Microvascular EC | 11.0 |
| | | none | |
| Primary Th1 act | 40.9 | Lung Microvascular EC | 12.0 |
| | | TNF alpha + IL-1 beta | |
| Primary Th2 act | 19.1 | Microvascular Dermal EC | 3.1 |
| | | none | |
| Primary Tr1 act | 51.4 | Microsvasular Dermal EC | 1.2 |
| | | TNF alpha + IL-1 beta | |
| Primary Th1 rest | 23.5 | Bronchial epithelium | 2.4 |
| | | TNF alpha + IL1 beta | |
| Primary Th2 rest | 11.8 | Small airway epithelium | 0.8 |
| | | none | |
| Primary Tr1 rest | 17.2 | Small airway epithelium | 9.6 |
| | | TNF alpha + IL-1 beta | |
| CD45RA CD4 | 1.6 | Coronery artery SMC rest | 5.5 |
| lymphocyte act | | | |
| CD45RO CD4 | 12.8 | Coronery artery SMC | 2.5 |
| lymphocyte act | | TNF alpha + IL-1 beta | |
| CD8 lymphocyte act | 1.0 | Astrocytes rest | 6.3 |
| Secondary CD8 | 2.7 | Astrocytes TNF alpha + | 1.8 |
| lymphocyte rest | | IL-1 beta | |
| Secondary CD8 | 3.4 | KU-812 (Basophil) rest | 1.8 |
| lymphocyte act | | | |
| CD4 lymphocyte none | 0.0 | KU-812 (Basophil) | 21.6 |
| | | PMA/ionomycin | |
| 2ry Th1/Th2/Tr1_anti- | 1.5 | CCD1106 (Keratinocytes) | 6.3 |
| CD95 CH11 | | none | |
| LAK cells rest | 0.7 | CCD1106 (Keratinocytes) | 1.0 |
| | | TNF alpha + IL-1 beta | |
| LAK cells IL-2 | 0.0 | Liver cirrhosis | 5.4 |
| LAK cells IL-2 + IL-12 | 0.0 | Lupus kidney | 0.9 |
| LAK cells IL-2 + IFN | 3.1 | NCI-H292 none | 34.2 |
| gamma | | | |
| LAK cells IL-2 + IL-18 | 2.4 | NCI-H292 IL-4 | 46.3 |
| LAK cells | 0.0 | NCI-H292 IL-9 | 29.7 |
| PMA/ionomycin | | | |
| NK Cells IL-2 rest | 1.3 | NCI-H292 IL-13 | 22.8 |
| Two Way MLR 3 day | 0.6 | NCI-H292 IFN gamma | 25.2 |
| Two Way MLR 5 day | 1.1 | HPAEC none | 18.0 |
| Two Way MLR 7 day | 3.1 | HPAEC TNF alpha + IL-1 | 19.9 |
| | | beta | |
| PBMC rest | 0.0 | Lung fibroblast none | 0.8 |
| PBMC PWM | 4.1 | Lung fibroblast TNF alpha + | 0.0 |
| | | IL-1 beta | |
| PBMC PHA-L | 9.9 | Lung fibroblast IL-4 | 8.9 |
| Ramos (B cell) none | 50.7 | Lung fibroblast IL-9 | 7.9 |
| Ramos (B cell) | 100.0 | Lung fibroblast IL-13 | 4.9 |
| ionomycin | | | |
| B lymphocytes PWM | 1.1 | Lung fibroblast IFN | 4.9 |
| | | gamma | |
| B lymphocytes CD40L | 3.4 | Dermal fibroblast | 42.6 |
| and IL-4 | | CCD1070 rest | |
| EOL-1 dbcAMP | 0.0 | Dermal fibroblast | 39.8 |
| | | CCD1070 TNF alpha | |
| EOL-1 dbcAMP | 0.0 | Dermal fibroblast | 13.4 |
| PMA/ionomycin | | CCD1070 IL-1 beta | |
| Dendritic cells none | 2.1 | Dermal fibroblast IFN | 2.0 |
| | | gamma | |
| Dendritic cells LPS | 0.0 | Dermal fibroblast IL-4 | 2.6 |
| Dendritic cells anti- | 0.6 | IBD Colitis 2 | 1.1 |
| CD40 | | | |
| Monocytes rest | 0.0 | IBD Crohn's | 2.2 |
| Monocytes LPS | 0.0 | Colon | 3.2 |
| Macrophages rest | 4.8 | Lung | 1.0 |
| Macrophages LPS | 2.4 | Thymus | 4.4 |
| HUVEC none | 16.5 | Kidney | 16.3 |
| HUVEC starved | 17.6 | | |

Panel 1.3D Summary: Ag2869 Expression of the NOV13a gene is low/undetectable in all samples on this panel (CTs>35). (Data not shown.) The amp plot indicates that there is a high probability of a probe failure.

Panel 2.2 Summary: Ag2869 Expression of the NOV13a gene is low/undetectable in all samples on this panel (CTs>35). (Data not shown.)

Panel 4D Summary: Ag2869 Expression of the NOV13a gene is widespread among the samples in this panel, with highest expression in the B cell line Ramos treated with ionomycin (CT=31.1). Lower but still significant levels of expression are seen in untreated Ramos B cells. B cells represent a principle component of immunity and contribute to the immune response in a number of important functional roles, including antibody production. For example, production of antibodies against self-antigens is a major component in autoimmune disorders such a systemic lupus erythematosus, with B cells playing a major role. Since B cells play an important role in autoimmunity, inflammatory processes and inflammatory cascades, therapeutic modulation of this gene product may reduce or eliminate the symptoms of patients suffering from asthma, allergies, chronic obstructive pulmonary disease, emphysema, Crohn's disease, ulcerative colitis, rheumatoid arthritis, psoriasis, osteoarthritis, and other autoimmune disorders including systemic lupus erythematosus.

Significant levels of expression are also seen in IL-4, IL-9, IL-13 and IFN gamma activated-NCI-H292 mucoepidermoid cells as well as untreated NCI-H292 cells. Moderate expression is also detected in both treated and untreated human pulmonary aortic endothelial cells The expression of this gene in cells derived from or within the lung suggests that this gene may be involved in normal conditions as well as pathological and inflammatory lung disorders that include chronic obstructive pulmonary disease, asthma, allergy and emphysema

NOV14

Expression of gene NOV14 was assessed using the primer-probe set Ag2870, described in Table JA. Results of the RTQ-PCR runs are shown in Tables JB and JC.

TABLE JA
Probe Name Ag2870

| | | | Start | |
|---|---|---|---|---|
| Primers | Sequence | Length | Position | SEQ ID NO: |
| Forward | 5′-ttctcctgagagcaagaagttg-3′ | 22 | 823 | 1030 |
| Probe | TET-5′-tgtgactcccatgttgaacccatta-3′-TAMRA | 26 | 865 | 1031 |
| Reverse | 5′-tcttcacctcgctatttctcaa-3′ | 22 | 899 | 1032 |

TABLE JB
Panel 1.3D

| | Rel. Exp. (%) Ag2870, | | Rel. Exp. (%) Ag2870, |
|---|---|---|---|
| Tissue Name | Run 167646334 | Tissue Name | Run 167646334 |
| Liver adenocarcinoma | 0.0 | Kidney (fetal) | 0.0 |
| Pancreas | 0.0 | Renal ca. 786-0 | 2.5 |
| Pancreatic ca. CAPAN2 | 0.0 | Renal ca. A498 | 0.0 |
| Adrenal gland | 0.0 | Renal ca. RXF 393 | 0.0 |
| Thyroid | 0.0 | Renal ca. ACHN | 0.0 |
| Salivary gland | 0.0 | Renal ca. UO-31 | 0.0 |
| Pituitary gland | 0.0 | Renal ca. TK-10 | 7.5 |
| Brain (fetal) | 0.0 | Liver | 0.0 |
| Brain (whole) | 0.0 | Liver (fetal) | 0.0 |
| Brain (amygdala) | 0.0 | Liver ca. | 0.0 |
| | | (hepatoblast) HepG2 | |
| Brain (cerebellum) | 0.0 | Lung | 0.0 |
| Brain (hippocampus) | 0.0 | Lung (fetal) | 0.0 |
| Brain (substantia nigra) | 0.0 | Lung ca. (small cell) | 0.0 |
| | | LX-1 | |
| Brain (thalamus) | 6.1 | Lung ca. (small cell) | 0.0 |
| | | NCI-H69 | |
| Cerebral Cortex | 0.0 | Lung ca. (s.cell var.) | 0.0 |
| | | SHP-77 | |
| Spinal cord | 0.0 | Lung ca. (large | 0.0 |
| | | cell)NCI-H460 | |
| glio/astro U87-MG | 20.0 | Lung ca. (non-sm. | 0.0 |
| | | cell) A549 | |
| glio/astro U-118-MG | 0.0 | Lung ca. (non-s.cell) | 0.0 |
| | | NCI-H23 | |
| astrocytoma SW1783 | 0.0 | Lung ca. (non-s.cell) | 0.0 |
| | | HOP-62 | |
| neuro*; met SK-N-AS | 0.0 | Lung ca. (non-s.cl) | 0.0 |
| | | NCI-H522 | |
| astrocytoma SF-539 | 0.0 | Lung ca. (squam.) | 0.0 |
| | | SW 900 | |
| astrocytoma SNB-75 | 0.0 | Lung ca. (squam.) | 0.0 |
| | | NCI-H596 | |
| glioma SNB-19 | 0.0 | Mammary gland | 0.0 |
| glioma U251 | 0.0 | Breast ca.* (pl.ef) | 0.0 |
| | | MCF-7 | |
| glioma SF-295 | 0.0 | Breast ca.* (pl.ef) | 3.8 |
| | | MDA-MB-231 | |
| Heart (fetal) | 0.0 | Breast ca.* (pl.ef) | 0.0 |
| | | T47D | |
| Heart | 0.0 | Breast ca. BT-549 | 0.0 |
| Skeletal muscle (fetal) | 0.0 | Breast ca. MDA-N | 0.0 |
| Skeletal muscle | 0.0 | Ovary | 0.0 |
| Bone marrow | 0.0 | Ovarian ca. OVCAR-3 | 0.0 |
| Thymus | 8.7 | Ovarian ca. OVCAR-4 | 0.0 |
| Spleen | 0.0 | Ovarian ca. OVCAR-5 | 28.9 |
| Lymph node | 0.0 | Ovarian ca. OVCAR-8 | 13.4 |
| Colorectal | 0.0 | Ovarian ca. IGROV-1 | 0.0 |
| Stomach | 0.0 | Ovarian ca.* (ascites) | 21.3 |
| | | SK-OV-3 | |
| Small intestine | 0.0 | Uterus | 0.0 |
| Colon ca. SW480 | 0.0 | Placenta | 0.0 |
| Colon ca.* | 100.0 | Prostate | 0.0 |
| SW620(SW480 met) | | | |
| Colon ca. HT29 | 0.0 | Prostate ca.* (bone | 0.0 |
| | | met)PC-3 | |
| Colon ca. HCT-116 | 0.0 | Testis | 5.0 |
| Colon ca. CaCo-2 | 0.0 | Melanoma | 0.0 |
| | | Hs688(A).T | |
| Colon ca. | 0.0 | Melanoma* (met) | 0.0 |
| tissue(ODO3866) | | Hs688(B).T | |
| Colon ca. HCC-2998 | 0.0 | Melanoma UACC-62 | 0.0 |
| Gastric ca.* (liver met) | 0.0 | Melanoma M14 | 0.0 |
| NCI-N87 | | | |
| Bladder | 0.0 | Melanoma LOX | 0.0 |
| | | IMVI | |
| Trachea | 0.0 | Melanoma* (met) | 33.9 |
| | | SK-MEL-5 | |
| Kidney | 0.0 | Adipose | 0.0 |

TABLE JC
Panel 4D

| | Rel. Exp. (%) | | Rel. Exp. (%) |
|---|---|---|---|
| | Ag2870, Run | | Ag2870, Run |
| Tissue Name | 164328103 | Tissue Name | 164328103 |
| Secondary Th1 act | 9.7 | HUVEC IL-1 beta | 9.3 |
| Secondary Th2 act | 30.4 | HUVEC IFN gamma | 11.6 |
| Secondary Tr1 act | 25.2 | HUVEC TNF alpha + IFN | 0.0 |
| | | gamma | |
| Secondary Th1 rest | 0.2 | HUVEC TNF alpha + IL4 | 8.2 |
| Secondary Th2 rest | 1.9 | HUVEC IL-11 | 0.0 |
| Secondary Tr1 rest | 2.0 | Lung Microvascular EC | 11.5 |
| | | none | |
| Primary Th1 act | 16.7 | Lung Microvascular EC | 3.6 |
| | | TNF alpha + IL-1 beta | |
| Primary Th2 act | 12.3 | Microvascular Dermal EC | 0.0 |
| | | none | |
| Primary Tr1 act | 29.1 | Microsvasular Dermal EC | 2.6 |
| | | TNF alpha + IL-1 beta | |
| Primary Th1 rest | 9.3 | Bronchial epithelium | 0.0 |
| | | TNF alpha + IL1 beta | |
| Primary Th2 rest | 1.1 | Small airway epithelium | 0.0 |
| | | none | |
| Primary Tr1 rest | 4.5 | Small airway epithelium | 13.3 |
| | | TNF alpha + IL-1 beta | |
| CD45RA CD4 | 3.2 | Coronery artery SMC rest | 1.7 |
| lymphocyte act | | | |
| CD45RO CD4 | 6.5 | Coronery artery SMC | 0.0 |
| lymphocyte act | | TNF alpha + IL-1 beta | |
| CD8 lymphocyte act | 1.6 | Astrocytes rest | 0.0 |
| Secondary CD8 | 0.0 | Astrocytes TNF alpha + | 0.0 |
| lymphocyte rest | | IL-1 beta | |
| Secondary CD8 | 1.4 | KU-812 (Basophil) rest | 2.4 |
| lymphocyte act | | | |
| CD4 lymphocyte none | 0.0 | KU-812 (Basophil) | 8.7 |
| | | PMA/ionomycin | |
| 2ry Th1/Th2/Tr1_anti- | 4.7 | CCD1106 (Keratinocytes) | 6.6 |
| CD95 CH11 | | none | |
| LAK cells rest | 0.0 | CCD1106 (Keratinocytes) | 0.0 |
| | | TNF alpha + IL-1 beta | |
| LAK cells IL-2 | 0.0 | Liver cirrhosis | 1.2 |
| LAK cells IL-2 + IL-12 | 0.0 | Lupus kidney | 0.0 |
| LAK cells IL-2 + IFN | 0.6 | NCI-H292 none | 30.8 |
| gamma | | | |
| LAK cells IL-2 + IL-18 | 0.0 | NCI-H292 IL-4 | 32.5 |
| LAK cells | 0.4 | NCI-H292 IL-9 | 26.8 |
| PMA/ionomycin | | | |
| NK Cells IL-2 rest | 0.0 | NCI-H292 IL-13 | 11.3 |
| Two Way MLR 3 day | 0.0 | NCI-H292 IFN gamma | 21.3 |
| Two Way MLR 5 day | 0.0 | HPAEC none | 9.2 |
| Two Way MLR 7 day | 0.0 | HPAEC TNF alpha + IL-1 | 8.0 |
| | | beta | |
| PBMC rest | 0.0 | Lung fibroblast none | 1.9 |
| PBMC PWM | 0.9 | Lung fibroblast TNF alpha + | 1.8 |
| | | IL-1 beta | |
| PBMC PHA-L | 5.0 | Lung fibroblast IL-4 | 7.5 |
| Ramos (B cell) none | 36.3 | Lung fibroblast IL-9 | 7.0 |
| Ramos (B cell) | 100.0 | Lung fibroblast IL-13 | 0.5 |
| ionomycin | | | |
| B lymphocytes PWM | 0.0 | Lung fibroblast IFN | 1.4 |
| | | gamma | |
| B lymphocytes CD40L | 3.1 | Dermal fibroblast | 26.8 |
| and IL-4 | | CCD1070 rest | |
| EOL-1 dbcAMP | 0.0 | Dermal fibroblast | 23.2 |
| | | CCD1070 TNF alpha | |
| EOL-1 dbcAMP | 0.0 | Dermal fibroblast | 14.6 |
| PMA/ionomycin | | CCD1070 IL-1 beta | |
| Dendritic cells none | 0.0 | Dermal fibroblast IFN | 0.0 |
| | | gamma | |
| Dendritic cells LPS | 0.0 | Dermal fibroblast IL-4 | 2.3 |
| Dendritic cells anti- | 0.0 | IBD Colitis 2 | 0.4 |
| CD40 | | | |
| Monocytes rest | 0.0 | IBD Crohn's | 0.0 |
| Monocytes LPS | 0.0 | Colon | 1.8 |
| Macrophages rest | 3.5 | Lung | 0.0 |
| Macrophages LPS | 0.0 | Thymus | 0.3 |
| HUVEC none | 12.9 | Kidney | 8.1 |
| HUVEC starved | 22.7 | | |

Panel 1.3D Summary: Ag2870 Expression of the NOV14 gene is restricted to a sample derived from a colon cancer cell line (CT=34.4). Thus, expression of this gene could be used to differentiate between this sample and other samples on this panel and as a marker to detect the presence of colon cancer. Furthermore, therapeutic modulation of the expression or function of this gene may be effective in the treatment of colon cancer.

Panel 2.2 Summary: Ag2870 Expression of the NOV14 gene is low/undetectable in all samples on this panel (CTs>35). (Data not shown.)

Panel 4D Summary: Ag2870 Expression of the NOV14 gene is highest in the B cell line Ramos treated with ionomycin (CT=30.2). Lower but still significant levels of expression are seen in untreated Ramos B cells. B cells represent a principle component of immunity and contribute to the immune response in a number of important functional roles, including antibody production. For example, production of antibodies against self-antigens is a major component in autoimmune disorders such a systemic lupus erythematosus, with B cells playing a major role. Since B cells play an important role in autoimmunity, inflammatory processes and inflammatory cascades, therapeutic modulation of this gene product may reduce or eliminate the symptoms of patients suffering from asthma, allergies, chronic obstructive pulmonary disease, emphysema, Crohn's disease, ulcerative colitis, rheumatoid arthritis, psoriasis, osteoarthritis, and other autoimmune disorders including systemic lupus erythematosus.

Significant levels of expression are also seen in IL-4, IL-9, IL-13, IFN gamma activated and untreated NCI-H292 mucoepidermoid cells, IL-4, IL-9, IL-13 and IFN gamma activated lung fibroblasts, human pulmonary aortic endothelial cells (treated and untreated), treated small airway epithelium and lung microvascular endothelial cells (treated and untreated). The expression of this gene in cells derived from or within the lung further suggests that this gene may be involved in normal conditions as well as pathological and inflammatory lung disorders that include chronic obstructive pulmonary disease, asthma, allergy and emphysema.

NOV15a and NOV15b

Expression of gene NOV15a and variant NOV15b was assessed using the primer-probe set Ag2875, described in Table KA. Results of the RTQ-PCR runs are shown in Table KB.

TABLE KA
Probe Name Ag2875

| | | | Start | |
|---|---|---|---|---|
| Primers | Sequence | Length | Position | SEQ ID NO: |
| Forward | 5′-gaacatcatctcctaccctgaa-3′ | 22 | 268 | 1033 |
| Probe | TET-5′-tgcatgactcagctctacttcttcctcg-3′-TAMRA | 28 | 290 | 1034 |
| Reverse | 5′-atgtgacactctgcaatagcaa-3′ | 22 | 321 | 1035 |

TABLE KB
Panel 4D

| | Rel. Exp. (%) | | Rel. Exp. (%) |
|---|---|---|---|
| | Ag2875, Run | | Ag2875, Run |
| Tissue Name | 164311029 | Tissue Name | 164311029 |
| Secondary Th1 act | 0.0 | HUVEC IL-1 beta | 0.0 |
| Secondary Th2 act | 0.0 | HUVEC IFN gamma | 3.0 |
| Secondary Tr1 act | 0.0 | HUVEC TNF alpha + IFN | 0.0 |
| | | gamma | |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC | 0.0 |
| | | none | |
| Primary Th1 act | 0.0 | Lung Microvascular EC | 0.0 |
| | | TNF alpha + IL-1 beta | |
| Primary Th2 act | 0.0 | Microvascular Dermal EC | 0.0 |
| | | none | |
| Primary Tr1 act | 0.0 | Microsvasular Dermal EC | 0.0 |
| | | TNF alpha + IL-1 beta | |
| Primary Th1 rest | 0.0 | Bronchial epithelium | 0.0 |
| | | TNF alpha + IL1 beta | |
| Primary Th2 rest | 0.0 | Small airway epithelium | 0.0 |
| | | none | |
| Primary Tr1 rest | 0.0 | Small airway epithelium | 0.0 |
| | | TNF alpha + IL-1 beta | |
| CD45RA CD4 | 0.0 | Coronery artery SMC rest | 0.0 |
| lymphocyte act | | | |
| CD45RO CD4 | 0.0 | Coronery artery SMC | 0.0 |
| lymphocyte act | | TNF alpha + IL-1 beta | |
| CD8 lymphocyte act | 0.0 | Astrocytes rest | 0.0 |
| Secondary CD8 | 0.0 | Astrocytes TNF alpha + | 0.0 |
| lymphocyte rest | | IL-1 beta | |
| Secondary CD8 | 0.0 | KU-812 (Basophil) rest | 0.0 |
| lymphocyte act | | | |
| CD4 lymphocyte none | 0.0 | KU-812 (Basophil) | 24.0 |
| | | PMA/ionomycin | |
| 2ry Th1/Th2/Tr1_anti- | 5.1 | CCD1106 (Keratinocytes) | 0.0 |
| CD95 CH11 | | none | |
| LAK cells rest | 0.0 | CCD1106 (Keratinocytes) | 0.0 |
| | | TNF alpha + IL-1 beta | |
| LAK cells IL-2 | 0.0 | Liver cirrhosis | 72.2 |
| LAK cells IL-2 + IL-12 | 0.0 | Lupus kidney | 0.0 |
| LAK cells IL-2 + IFN | 0.0 | NCI-H292 none | 20.7 |
| gamma | | | |
| LAK cells IL-2 + IL-18 | 0.0 | NCI-H292 IL-4 | 0.0 |
| LAK cells | 0.0 | NCI-H292 IL-9 | 0.0 |
| PMA/ionomycin | | | |
| NK Cells IL-2 rest | 0.0 | NCI-H292 IL-13 | 0.0 |
| Two Way MLR 3 day | 0.0 | NCI-H292 IFN gamma | 0.0 |
| Two Way MLR 5 day | 0.0 | HPAEC none | 0.0 |
| Two Way MLR 7 day | 0.0 | HPAEC TNF alpha + IL-1 | 1.4 |
| | | beta | |
| PBMC rest | 0.0 | Lung fibroblast none | 0.0 |
| PBMC PWM | 0.0 | Lung fibroblast TNF | 0.0 |
| | | alpha + IL-1 beta | |
| PBMC PHA-L | 0.0 | Lung fibroblast IL-4 | 0.0 |
| Ramos (B cell) none | 0.0 | Lung fibroblast IL-9 | 0.0 |
| Ramos (B cell) | 0.0 | Lung fibroblast IL-13 | 0.0 |
| ionomycin | | | |
| B lymphocytes PWM | 0.0 | Lung fibroblast IFN | 3.2 |
| | | gamma | |
| B lymphocytes CD40L | 0.0 | Dermal fibroblast | 0.0 |
| and IL-4 | | CCD1070 rest | |
| EOL-1 dbcAMP | 3.1 | Dermal fibroblast | 0.0 |
| | | CCD1070 TNF alpha | |
| EOL-1 dbcAMP | 0.0 | Dermal fibroblast | 0.0 |
| PMA/ionomycin | | CCD1070 IL-1 beta | |
| Dendritic cells none | 33.2 | Dermal fibroblast IFN | 0.0 |
| | | gamma | |
| Dendritic cells LPS | 2.4 | Dermal fibroblast IL-4 | 1.9 |
| Dendritic cells anti- | 100.0 | IBD Colitis 2 | 16.3 |
| CD40 | | | |
| Monocytes rest | 0.0 | IBD Crohn's | 6.7 |
| Monocytes LPS | 0.0 | Colon | 0.0 |
| Macrophages rest | 44.1 | Lung | 21.8 |
| Macrophages LPS | 0.0 | Thymus | 0.0 |
| HUVEC none | 3.1 | Kidney | 0.0 |
| HUVEC starved | 0.0 | | |

Panel 1.3D Summary: Ag2875 Results from one experiment with the NOV15a gene are not included. The amp plot indicates that there were experimental difficulties with this run.

Panel 2.2 Summary: Ag2875 Expression of the NOV15a gene is low/undetectable in all samples on this panel (CTs>35). (Data not shown.)

Panel 4D Summary: Ag2875 Highest expression of the NOV15a is in anti-CD40 treated dendritic cells (CT=33.2), with much lower expression in untreated dendritic cells. Thus, this gene product may be important in dendritic cell activation. Significant expression of this gene is also seen in liver cirrhosis. This gene encodes a putative GPCR; therefore, antibodies or small molecule therapeutics could reduce or inhibit fibrosis that occurs in liver cirrhosis. In addition, antibodies to this putative GPCR could also be used for the diagnosis of liver cirrhosis. In addition, significant expression of this gene is seen in resting macrophages. The putative GPCR encoded for by this transcript may therefore be important in macrophage detection of chemokine gradients and trafficking into specific sites within a tissue and in activation. Antibody or protein therapeutics designed against the protein encoded for by this transcript could reduce or inhibit inflammation in asthma, emphysema, allergy, psoriasis, arthritis, or any other condition in which macrophage localization/activation is important.

NOV16A: Olfactory Receptor

Expression of gene NOV16a was assessed using the primer-probe set Ag2876, described in Table LA. Results of the RTQ-PCR runs are shown in Tables LB and LC.

TABLE LA
Probe Name Ag2876

| | | | Start | |
|---|---|---|---|---|
| Primers | Sequence | Length | Position | SEQ ID NO: |
| Forward | 5′-acatcatctcctaccctgaatg-3′ | 22 | 273 | 1036 |
| Probe | TET-5′-catgactcagctttacttcttcctcatt-3′-TAMRA | 28 | 295 | 1037 |
| Reverse | 5′-tacagccaacatgtgacactct-3′ | 22 | 334 | 1038 |

TABLE LB
Panel 1.3D

| | Rel. Exp. (%) Ag2876, | | Rel. Exp. (%) Ag2876, |
|---|---|---|---|
| Tissue Name | Run 167646343 | Tissue Name | Run 167646343 |
| Liver adenocarcinoma | 0.0 | Kidney (fetal) | 0.0 |
| Pancreas | 0.0 | Renal ca. 786-0 | 0.0 |
| Pancreatic ca. CAPAN2 | 0.0 | Renal ca. A498 | 0.0 |
| Adrenal gland | 0.0 | Renal ca. RXF 393 | 0.0 |
| Thyroid | 0.0 | Renal ca. ACHN | 0.0 |
| Salivary gland | 0.0 | Renal ca. UO-31 | 0.0 |
| Pituitary gland | 0.0 | Renal ca. TK-10 | 0.0 |
| Brain (fetal) | 0.0 | Liver | 0.0 |
| Brain (whole) | 0.0 | Liver (fetal) | 0.0 |
| Brain (amygdala) | 0.0 | Liver ca. | 0.0 |
| | | (hepatoblast) HepG2 | |
| Brain (cerebellum) | 0.0 | Lung | 0.0 |
| Brain (hippocampus) | 0.0 | Lung (fetal) | 0.0 |
| Brain (substantia nigra) | 0.0 | Lung ca. (small cell) | 0.0 |
| | | LX-1 | |
| Brain (thalamus) | 1.7 | Lung ca. (small cell) | 0.0 |
| | | NCI-H69 | |
| Cerebral Cortex | 0.0 | Lung ca. (s.cell var.) | 0.0 |
| | | SHP-77 | |
| Spinal cord | 0.0 | Lung ca. (large | 0.0 |
| | | cell)NCI-H460 | |
| glio/astro U87-MG | 0.0 | Lung ca. (non-sm. | 0.0 |
| | | cell) A549 | |
| glio/astro U-118-MG | 0.0 | Lung ca. (non-s.cell) | 0.0 |
| | | NCI-H23 | |
| astrocytoma SW1783 | 0.0 | Lung ca. (non-s.cell) | 0.0 |
| | | HOP-62 | |
| neuro*; met SK-N-AS | 0.0 | Lung ca. (non-s.cl) | 0.0 |
| | | NCI-H522 | |
| astrocytoma SF-539 | 0.0 | Lung ca. (squam.) | 0.0 |
| | | SW 900 | |
| astrocytoma SNB-75 | 0.0 | Lung ca. (squam.) | 0.0 |
| | | NCI-H596 | |
| glioma SNB-19 | 0.0 | Mammary gland | 0.0 |
| glioma U251 | 1.4 | Breast ca.* (pl.ef) | 19.2 |
| | | MCF-7 | |
| glioma SF-295 | 2.6 | Breast ca.* (pl.ef) | 0.0 |
| | | MDA-MB-231 | |
| Heart (fetal) | 0.0 | Breast ca.* (pl.ef) | 100.0 |
| | | T47D | |
| Heart | 0.0 | Breast ca. BT-549 | 0.0 |
| Skeletal muscle (fetal) | 0.0 | Breast ca. MDA-N | 0.0 |
| Skeletal muscle | 2.1 | Ovary | 0.0 |
| Bone marrow | 0.0 | Ovarian ca. OVCAR-3 | 0.0 |
| Thymus | 0.0 | Ovarian ca. OVCAR-4 | 0.0 |
| Spleen | 0.0 | Ovarian ca. OVCAR-5 | 0.0 |
| Lymph node | 0.0 | Ovarian ca. OVCAR-8 | 0.0 |
| Colorectal | 2.0 | Ovarian ca. IGROV-1 | 0.0 |
| Stomach | 0.0 | Ovarian ca.* (ascites) | 0.0 |
| | | SK-OV-3 | |
| Small intestine | 0.0 | Uterus | 0.0 |
| Colon ca. SW480 | 0.0 | Placenta | 0.0 |
| Colon ca.* | 0.0 | Prostate | 0.0 |
| SW620(SW480 met) | | | |
| Colon ca. HT29 | 0.0 | Prostate ca.* (bone | 3.6 |
| | | met)PC-3 | |
| Colon ca. HCT-116 | 0.0 | Testis | 8.5 |
| Colon ca. CaCo-2 | 0.0 | Melanoma | 0.0 |
| | | Hs688(A).T | |
| Colon ca. | 0.0 | Melanoma* (met) | 0.0 |
| tissue(ODO3866) | | Hs688(B).T | |
| Colon ca. HCC-2998 | 0.0 | Melanoma UACC-62 | 24.7 |
| Gastric ca.* (liver met) | 0.0 | Melanoma M14 | 0.0 |
| NCI-N87 | | | |
| Bladder | 0.0 | Melanoma LOX | 0.0 |
| | | IMVI | |
| Trachea | 0.0 | Melanoma* (met) | 0.0 |
| | | SK-MEL-5 | |
| Kidney | 0.0 | Adipose | 0.0 |

TABLE LC
Panel 4D

| | Rel. Exp. (%) | | Rel. Exp. (%) |
|---|---|---|---|
| | Ag2876, Run | | Ag2876, Run |
| Tissue Name | 164328133 | Tissue Name | 164328133 |
| Secondary Th1 act | 0.0 | HUVEC IL-1 beta | 0.0 |
| Secondary Th2 act | 0.0 | HUVEC IFN gamma | 0.0 |
| Secondary Tr1 act | 0.0 | HUVEC TNF alpha + IFN | 0.0 |
| | | gamma | |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC | 0.0 |
| | | none | |
| Primary Th1 act | 0.0 | Lung Microvascular EC | 0.0 |
| | | TNF alpha + IL-1 beta | |
| Primary Th2 act | 0.0 | Microvascular Dermal EC | 0.0 |
| | | none | |
| Primary Tr1 act | 0.0 | Microsvasular Dermal EC | 0.0 |
| | | TNF alpha + IL-1 beta | |
| Primary Th1 rest | 0.0 | Bronchial epithelium | 0.0 |
| | | TNF alpha + IL1 beta | |
| Primary Th2 rest | 0.0 | Small airway epithelium | 2.1 |
| | | none | |
| Primary Tr1 rest | 0.0 | Small airway epithelium | 0.0 |
| | | TNF alpha + IL-1 beta | |
| CD45RA CD4 | 0.0 | Coronery artery SMC rest | 0.0 |
| lymphocyte act | | | |
| CD45RO CD4 | 0.0 | Coronery artery SMC | 0.0 |
| lymphocyte act | | TNF alpha + IL-1 beta | |
| CD8 lymphocyte act | 9.7 | Astrocytes rest | 0.0 |
| Secondary CD8 | 0.0 | Astrocytes TNF alpha + | 0.0 |
| lymphocyte rest | | IL-1 beta | |
| Secondary CD8 | 0.0 | KU-812 (Basophil) rest | 0.0 |
| lymphocyte act | | | |
| CD4 lymphocyte none | 0.0 | KU-812 (Basophil) | 30.6 |
| | | PMA/ionomycin | |
| 2ry Th1/Th2/Tr1_anti- | 1.5 | CCD1106 (Keratinocytes) | 0.0 |
| CD95 CH11 | | none | |
| LAK cells rest | 0.0 | CCD1106 (Keratinocytes) | 0.0 |
| | | TNF alpha + IL-1 beta | |
| LAK cells IL-2 | 0.0 | Liver cirrhosis | 100.0 |
| LAK cells IL-2 + IL-12 | 0.0 | Lupus kidney | 0.0 |
| LAK cells IL-2 + IFN | 0.0 | NCI-H292 none | 0.0 |
| gamma | | | |
| LAK cells IL-2 + IL-18 | 0.0 | NCI-H292 IL-4 | 0.0 |
| LAK cells | 0.0 | NCI-H292 IL-9 | 0.0 |
| PMA/ionomycin | | | |
| NK Cells IL-2 rest | 0.0 | NCI-H292 IL-13 | 0.0 |
| Two Way MLR 3 day | 0.0 | NCI-H292 IFN gamma | 8.8 |
| Two Way MLR 5 day | 0.0 | HPAEC none | 0.0 |
| Two Way MLR 7 day | 0.0 | HPAEC TNF alpha + IL-1 | 0.0 |
| | | beta | |
| PBMC rest | 0.0 | Lung fibroblast none | 0.0 |
| PBMC PWM | 0.0 | Lung fibroblast TNF alpha + | 0.0 |
| | | IL-1 beta | |
| PBMC PHA-L | 0.0 | Lung fibroblast IL-4 | 0.0 |
| Ramos (B cell) none | 0.0 | Lung fibroblast IL-9 | 4.8 |
| Ramos (B cell) | 0.0 | Lung fibroblast IL-13 | 0.0 |
| ionomycin | | | |
| B lymphocytes PWM | 0.0 | Lung fibroblast IFN | 15.6 |
| | | gamma | |
| B lymphocytes CD40L | 0.0 | Dermal fibroblast | 0.0 |
| and IL-4 | | CCD1070 rest | |
| EOL-1 dbcAMP | 0.0 | Dermal fibroblast | 0.0 |
| | | CCD1070 TNF alpha | |
| EOL-1 dbcAMP | 0.0 | Dermal fibroblast | 5.4 |
| PMA/ionomycin | | CCD1070 IL-1 beta | |
| Dendritic cells none | 10.2 | Dermal fibroblast IFN | 0.0 |
| | | gamma | |
| Dendritic cells LPS | 0.0 | Dermal fibroblast IL-4 | 19.3 |
| Dendritic cells anti- | 18.8 | IBD Colitis 2 | 15.3 |
| CD40 | | | |
| Monocytes rest | 0.0 | IBD Crohn's | 2.2 |
| Monocytes LPS | 20.6 | Colon | 4.1 |
| Macrophages rest | 14.2 | Lung | 21.0 |
| Macrophages LPS | 0.0 | Thymus | 0.0 |
| HUVEC none | 0.0 | Kidney | 0.0 |
| HUVEC starved | 0.0 | | |

Panel 1.3D Summary: Ag2876 Expression of the NOV16a gene is restricted to a sample derived from a breast cancer cell line (CT=32.5). Thus, expression of this gene could be used to differentiate between this sample and other samples on this panel and as a marker to detect the presence of breast cancer. Furthermore, therapeutic modulation of the expression or function of this gene may be effective in the treatment of breast cancer.

Panel 4D Summary: Ag2876 Significant expression of the NOV16a gene is detected in a liver cirrhosis sample (CT=33.5). Furthermore, expression of this gene is not detected in normal liver in Panel 1.3D, suggesting that its expression is unique to liver cirrhosis. This gene encodes a putative GPCR; therefore, antibodies or small molecule therapeutics could reduce or inhibit fibrosis that occurs in liver cirrhosis. In addition, antibodies to this putative GPCR could also be used for the diagnosis of liver cirrhosis.

NOV17a and NOV17b

Expression of gene NOV17a and variant NOV17b was assessed using the primer-probe set Ag2969, described in Table MA. Results of the RTQ-PCR runs are shown in Tables MB and MC.

TABLE MA
Probe Name Ag2969

| | | | Start | |
|---|---|---|---|---|
| Primers | Sequence | Length | Position | SEQ ID NO: |
| Forward | 5′-cacccatgtatttcctgcttag-3′ | 22 | 184 | 1039 |
| Probe | TET-5′-tcagctctccctcattgacctaatt-3′-TAMRA | 26 | 206 | 1040 |
| Reverse | 5′-tcagaagccatcttaggaacaa-3′ | 22 | 244 | 1041 |

TABLE MB
CNS_neurodegeneration_v1.0

| | Rel. Exp. (%) Ag2969, | | Rel. Exp. (%) Ag2969, |
|---|---|---|---|
| Tissue Name | Run 209778982 | Tissue Name | Run 209778982 |
| AD 1 Hippo | 0.5 | Control (Path) 3 | 0.5 |
| | | Temporal Ctx | |
| AD 2 Hippo | 9.1 | Control (Path) 4 | 23.7 |
| | | Temporal Ctx | |
| AD 3 Hippo | 0.7 | AD 1 Occipital Ctx | 4.2 |
| AD 4 Hippo | 1.6 | AD 2 Occipital Ctx | 0.0 |
| | | (Missing) | |
| AD 5 Hippo | 100.0 | AD 3 Occipital Ctx | 1.9 |
| AD 6 Hippo | 24.3 | AD 4 Occipital Ctx | 9.5 |
| Control 2 Hippo | 4.7 | AD 5 Occipital Ctx | 17.8 |
| Control 4 Hippo | 2.1 | AD 6 Occipital Ctx | 14.5 |
| Control (Path) 3 | 1.1 | Control 1 Occipital | 0.8 |
| Hippo | | Ctx | |
| AD 1 Temporal Ctx | 2.0 | Control 2 Occipital | 29.7 |
| | | Ctx | |
| AD 2 Temporal Ctx | 15.5 | Control 3 Occipital | 13.9 |
| | | Ctx | |
| AD 3 Temporal Ctx | 2.4 | Control 4 Occipital | 1.0 |
| | | Ctx | |
| AD 4 Temporal Ctx | 9.7 | Control (Path) 1 | 65.5 |
| | | Occipital Ctx | |
| AD 5 Inf Temporal | 91.4 | Control (Path) 2 | 4.8 |
| Ctx | | Occipital Ctx | |
| AD 5 Sup Temporal | 27.9 | Control (Path) 3 | 0.0 |
| Ctx | | Occipital Ctx | |
| AD 6 Inf Temporal | 42.9 | Control (Path) 4 | 13.6 |
| Ctx | | Occipital Ctx | |
| AD 6 Sup Temporal | 39.2 | Control 1 Parietal | 1.2 |
| Ctx | | Ctx | |
| Control 1 Temporal | 0.0 | Control 2 Parietal | 28.9 |
| Ctx | | Ctx | |
| Control 2 Temporal | 11.8 | Control 3 Parietal | 9.6 |
| Ctx | | Ctx | |
| Control 3 Temporal | 9.3 | Control (Path) 1 | 48.0 |
| Ctx | | Parietal Ctx | |
| Control 3 Temporal | 2.5 | Control (Path) 2 | 12.0 |
| Ctx | | Parietal Ctx | |
| Control (Path) 1 | 38.4 | Control (Path) 3 | 0.0 |
| Temporal Ctx | | Parietal Ctx | |
| Control (Path) 2 | 12.2 | Control (Path) 4 | 41.2 |
| Temporal Ctx | | Parietal Ctx | |

TABLE MC
Panel 4D

| | Rel. Exp. (%) | | Rel. Exp. (%) |
|---|---|---|---|
| | Ag2969, Run | | Ag2969, Run |
| Tissue Name | 164402668 | Tissue Name | 164402668 |
| Secondary Th1 act | 0.0 | HUVEC IL-1 beta | 0.0 |
| Secondary Th2 act | 3.0 | HUVEC IFN gamma | 2.8 |
| Secondary Tr1 act | 0.0 | HUVEC TNF alpha + IFN | 0.0 |
| | | gamma | |
| Secondary Th1 rest | 6.1 | HUVEC TNF alpha + IL4 | 0.0 |
| Secondary Th2 rest | 4.1 | HUVEC IL-11 | 0.0 |
| Secondary Tr1 rest | 0.0 | Lung Microvascular EC | 0.0 |
| | | none | |
| Primary Th1 act | 0.0 | Lung Microvascular EC | 0.0 |
| | | TNF alpha + IL-1 beta | |
| Primary Th2 act | 0.0 | Microvascular Dermal EC | 0.0 |
| | | none | |
| Primary Tr1 act | 0.0 | Microsvasular Dermal EC | 0.0 |
| | | TNF alpha + IL-1 beta | |
| Primary Th1 rest | 10.5 | Bronchial epithelium | 0.0 |
| | | TNF alpha + IL1 beta | |
| Primary Th2 rest | 13.6 | Small airway epithelium | 0.0 |
| | | none | |
| Primary Tr1 rest | 7.9 | Small airway epithelium | 0.0 |
| | | TNF alpha + IL-1 beta | |
| CD45RA CD4 | 0.0 | Coronery artery SMC rest | 2.0 |
| lymphocyte act | | | |
| CD45RO CD4 | 7.5 | Coronery artery SMC | 3.4 |
| lymphocyte act | | TNF alpha + IL-1 beta | |
| CD8 lymphocyte act | 4.9 | Astrocytes rest | 0.0 |
| Secondary CD8 | 4.0 | Astrocytes TNF alpha + | 0.0 |
| lymphocyte rest | | IL-1 beta | |
| Secondary CD8 | 0.0 | KU-812 (Basophil) rest | 8.5 |
| lymphocyte act | | | |
| CD4 lymphocyte none | 16.7 | KU-812 (Basophil) | 51.1 |
| | | PMA/ionomycin | |
| 2ry Th1/Th2/Tr1_anti- | 3.2 | CCD1106 (Keratinocytes) | 0.0 |
| CD95 CH11 | | none | |
| LAK cells rest | 23.2 | CCD1106 (Keratinocytes) | 0.0 |
| | | TNF alpha + IL-1 beta | |
| LAK cells IL-2 | 29.3 | Liver cirrhosis | 18.6 |
| LAK cells IL-2 + IL-12 | 22.2 | Lupus kidney | 0.0 |
| LAK cells IL-2 + IFN | 28.5 | NCI-H292 none | 0.0 |
| gamma | | | |
| LAK cells IL-2 + IL-18 | 21.0 | NCI-H292 IL-4 | 0.0 |
| LAK cells | 0.0 | NCI-H292 IL-9 | 0.0 |
| PMA/ionomycin | | | |
| NK Cells IL-2 rest | 7.2 | NCI-H292 IL-13 | 0.0 |
| Two Way MLR 3 day | 51.4 | NCI-H292 IFN gamma | 0.0 |
| Two Way MLR 5 day | 18.4 | HPAEC none | 0.0 |
| Two Way MLR 7 day | 2.9 | HAPEC TNF alpha + IL-1 | 0.0 |
| | | beta | |
| PBMC rest | 0.0 | Lung fibroblast none | 0.0 |
| PBMC PWM | 45.7 | Lung fibroblast TNF alpha + | 0.0 |
| | | IL-1 beta | |
| PBMC PHA-L | 13.8 | Lung fibroblast IL-4 | 0.0 |
| Ramos (B cell) none | 0.0 | Lung fibroblast IL-9 | 0.0 |
| Ramos (B cell) | 5.4 | Lung fibroblast IL-13 | 0.0 |
| ionomycin | | | |
| B lymphocytes PWM | 12.0 | Lung fibroblast IFN | 0.0 |
| | | gamma | |
| B lymphocytes CD40L | 39.2 | Dermal fibroblast | 0.0 |
| and IL-4 | | CCD1070 rest | |
| EOL-1 dbcAMP | 1.1 | Dermal fibroblast | 0.0 |
| | | CCD1070 TNF alpha | |
| EOL-1 dbcAMP | 0.0 | Dermal fibroblast | 0.0 |
| PMA/ionomycin | | CCD1070 IL-1 beta | |
| Dendritic cells none | 4.2 | Dermal fibroblast IFN | 0.0 |
| | | gamma | |
| Dendritic cells LPS | 5.0 | Dermal fibroblast IL-4 | 0.0 |
| Dendritic cells anti- | 6.4 | IBD Colitis 2 | 11.1 |
| CD40 | | | |
| Monocytes rest | 1.5 | IBD Crohn's | 0.0 |
| Monocytes LPS | 10.7 | Colon | 0.0 |
| Macrophages rest | 11.4 | Lung | 2.4 |
| Macrophages LPS | 3.7 | Thymus | 0.0 |
| HUVEC none | 0.0 | Kidney | 100.0 |
| HUVEC starved | 0.0 | | |

CNS_neurodegeneration_v1.0 Summary: Ag2969 The NOV17a gene represents a novel G-protein coupled receptor (GPCR) with expression in the brain. The GPCR family of receptors contains a large number of neurotransmitter receptors, including the dopamine, serotonin, a and b-adrenergic, acetylcholine muscarinic, histamine, peptide, and metabotropic glutamate receptors. GPCRs are excellent drug targets in various neurologic and psychiatric diseases. All antipsychotics have been shown to act at the dopamine D2 receptor; similarly novel antipsychotics also act at the serotonergic receptor, and often the muscarinic and adrenergic receptors as well. While the majority of antidepressants can be classified as selective serotonin reuptake inhibitors, blockade of the 5-HT1A and a2 adrenergic receptors increases the effects of these drugs. The GPCRs are also of use as drug targets in the treatment of stroke. Blockade of the glutamate receptors may decrease the neuronal death resulting from excitotoxicity; further more the purinergic receptors have also been implicated as drug targets in the treatment of cerebral ischemia. The b-adrenergic receptors have been implicated in the treatment of ADHD with Ritalin, while the a-adrenergic receptors have been implicated in memory. Therefore this gene may be of use as a small molecule target for the treatment of any of the described diseases.

In addition, this panel shows that this GPCR is upregulated in the temporal cortex of Alzheimer's disease patients. Therefore, blockade of this receptor may be of use in the treatment of this disease and decrease neuronal death.

References:

El Yacoubi M, Ledent C, Parmentier M, Bertorelli R, Ongini E, Costentin J, Vaugeois J M. Adenosine A2A receptor antagonists are potential antidepressants: evidence based on pharmacology and A2A receptor knockout mice. Br J Pharmacol September 2001;134(1):68-77

1. Adenosine, an ubiquitous neuromodulator, and its analogues have been shown to produce ‘depressant’ effects in animal models believed to be relevant to depressive disorders, while adenosine receptor antagonists have been found to reverse adenosine-mediated ‘depressant’ effect. 2. We have designed studies to assess whether adenosine A2A receptor antagonists, or genetic inactivation of the receptor would be effective in established screening procedures, such as tail suspension and forced swim tests, which are predictive of clinical antidepressant activity. 3. Adenosine A2A receptor knockout mice were found to be less sensitive to ‘depressant’ challenges than their wildtype littermates. Consistently, the adenosine A2A receptor blockers SCH 58261 (1-10 mg kg(−1), i.p.) and KW 6002 (0.1-10 mg kg(−1), p.o.) reduced the total immobility time in the tail suspension test. 4. The efficacy of adenosine A2A receptor antagonists in reducing immobility time in the tail suspension test was confirmed and extended in two groups of mice. Specifically, SCH 58261 (1-10 mg kg(−1)) and ZM 241385 (15-60 mg kg(−1)) were effective in mice previously screened for having high immobility time, while SCH 58261 at 10 mg kg(−1) reduced immobility of mice that were selectively bred for their spontaneous ‘helplessness’ in this assay. 5. Additional experiments were carried out using the forced swim test. SCH 58261 at 10 mg kg(−1) reduced the immobility time by 61%, while KW 6002 decreased the total immobility time at the doses of 1 and 10 mg kg(−1) by 75 and 79%, respectively. 6. Administration of the dopamine D2 receptor antagonist haloperidol (50-200 microg kg(−1) i.p.) prevented the antidepressant-like effects elicited by SCH 58261 (10 mg kg(−1) i.p.) in forced swim test whereas it left unaltered its stimulant motor effects. 7. In conclusion, these data support the hypothesis that A2A receptor antagonists prolong escape-directed behaviour in two screening tests for antidepressants. Altogether the results support the hypothesis that blockade of the adenosine A2A receptor might be an interesting target for the development of effective antidepressant agents.

Blier P. Pharmacology of rapid-onset antidepressant treatment strategies. Clin Psychiatry 2001;62 Suppl 15:12-7

Although selective serotonin reuptake inhibitors (SSRIs) block serotonin (5-HT) reuptake rapidly, their therapeutic action is delayed. The increase in synaptic 5-HT activates feedback mechanisms mediated by 5-HT1A (cell body) and 5-HT1 B (terminal) autoreceptors, which, respectively, reduce the firing in 5-HT neurons and decrease the amount of 5-HT released per action potential resulting in attenuated 5-HT neurotransmission. Long-term treatment desensitizes the inhibitory 5-HT1 autoreceptors, and 5-HT neurotransmission is enhanced. The time course of these events is similar to the delay of clinical action. The addition of pindolol, which blocks 5-HT1A receptors, to SSRI treatment decouples the feedback inhibition of 5-HT neuron firing and accelerates and enhances the antidepressant response. The neuronal circuitry of the 5-HT and norepinephrine (NE) systems and their connections to forebrain areas believed to be involved in depression has been dissected. The firing of 5-HT neurons in the raphe nuclei is driven, at least partly, by alpha1-adrenoceptor-mediated excitatory inputs from NE neurons. Inhibitory alpha2-adrenoceptors on the NE neuroterminals form part of a feedback control mechanism. Mirtazapine, an antagonist at alpha2-adrenoceptors, does not enhance 5-HT neurotransmission directly but disinhibits the NE activation of 5-HT neurons and thereby increases 5-HT neurotransmission by a mechanism that does not require a time-dependent desensitization of receptors. These neurobiological phenomena may underlie the apparently faster onset of action of mirtazapine compared with the SSRIs.

Tranquillini M E, Reggiani A. Glycine-site antagonists and stroke. Expert Opin Investig Drugs November 1999;8(11):1837-1848

The excitatory amino acid, (S)-glutamic acid, plays an important role in controlling many neuronal processes. Its action is mediated by two main groups of receptors: the ionotropic receptors (which include NMDA, AMPA and kainic acid subtypes) and the metabotropic receptors (mGluR(1-8)) mediating G-protein coupled responses. This review focuses on the strychnine insensitive glycine binding site located on the NMDA receptor channel, and on the possible use of selective antagonists for the treatment of stroke. Stroke is a devastating disease caused by a sudden vascular accident. Neurochemically, a massive release of glutamate occurs in neuronal tissue; this overactivates the NMDA receptor, leading to increased intracellular calcium influx, which causes neuronal cell death through necrosis. NMDA receptor activation strongly depends upon the presence of glycine as a co-agonist. Therefore, the administration of a glycine antagonist can block overactivation of NMDA receptors, thus preserving neurones from damage. The glycine antagonists currently identified can be divided into five main categories depending on their chemical structure: indoles, tetrahydroquinolines, benzoazepines, quinoxalinediones and pyrida-zinoquinolines.

Monopoli A, Lozza G, Forlani A, Mattavelli A, Ongini E. Blockade of adenosine A2A receptors by SCH 58261 results in neuroprotective effects in cerebral ischaemia in rats. Neuroreport Dec. 1, 1998;9(17):3955-9

Blockade of adenosine receptors can reduce cerebral infarct size in the model of global ischaemia. Using the potent and selective A2A adenosine receptor antagonist, SCH 58261, we assessed whether A2A receptors are involved in the neuronal damage following focal cerebral ischaemia as induced by occluding the left middle cerebral artery. SCH 58261 (0.01 mg/kg either i.p. or i.v.) administered to normotensive rats 10 min after ischaemia markedly reduced cortical infarct volume as measured 24 h later (30% vs controls, p<0.05). Similar effects were observed when SCH 58261 (0.01 mg/kg, i.p.) was administered to hypertensive rats (28% infarct volume reduction vs controls, p<0.05). Neuroprotective properties of SCH 58261 administered after ischaemia indicate that blockade of A2A adenosine receptors is a potentially useful biological target for the reduction of brain injury.

Panel 1.3D Summary: Ag2878 Expression of the NOV17a gene is low/undetectable in all samples on this panel (CTs>35). (Data not shown.)

Panel 4D Summary: Ag2878 Expression of the NOV17a gene is restricted to a few samples in this panel, with highest expression in the kidney (CT=33.1). Thus, the putative GPCR encoded for by this gene could allow cells within the kidney to respond to specific microenvironmental signals (For example, ref. 1). Therefore, antibody or small molecule therapies designed with the protein encoded for by this gene could modulate kidney function and be important in the treatment of inflammatory or autoimmune diseases that affect the kidney, including lupus and glomerulonephritis.

Furthermore, significant levels of expression are also seen in the PMA and ionomycin treated basophil cell line KU-812. GPCR-type receptors are important in multiple physiological responses mediated by basophils (ref. 2). Therefore, antibody or small molecule therapies designed with the protein encoded for by this gene could also block or inhibit inflammation or tissue damage due to basophil activation in response to asthma, allergies, hypersensitivity reactions, psoriasis, and viral infections.

References:

1. Mark M. D., Wittemann S., Herlitze S. (2000) G protein modulation of recombinant P/Q-type calcium channels by regulators of G protein signalling proteins. J. Physiol. 528 Pt 1: 65-77.

1. Fast synaptic transmission is triggered by the activation of presynaptic Ca2+ channels which can be inhibited by Gbetagamma subunits via G protein-coupled receptors (GPCR). Regulators of G protein signalling (RGS) proteins are GTPase-accelerating proteins (GAPs), which are responsible for >100-fold increases in the GTPase activity of G proteins and might be involved in the regulation of presynaptic Ca2+ channels. In this study we investigated the effects of RGS2 on G protein modulation of recombinant P/Q-type channels expressed in a human embryonic kidney (HEK293) cell line using whole-cell recordings. 2. RGS2 markedly accelerates transmitter-mediated inhibition and recovery from inhibition of Ba2+ currents (IBa) through P/Q-type channels heterologously expressed with the muscarinic acetylcholine receptor M2 (mAChR M2). 3. Both RGS2 and RGS4 modulate the prepulse facilitation properties of P/Q-type Ca2+ channels. G protein reinhibition is accelerated, while release from inhibition is slowed. These kinetics depend on the availability of G protein alpha and betagamma subunits which is altered by RGS proteins. 4. RGS proteins unmask the Ca2+ channel beta subunit modulation of Ca2+ channel G protein inhibition. In the presence of RGS2, P/Q-type channels containing the beta2a and beta3 subunits reveal significantly altered kinetics of G protein modulation and increased facilitation compared to Ca2+ channels coexpressed with the beta1b or beta4 subunit.

PMID: 11018106

2. Heinemann A., Hartnell A., Stubbs V. E., Murakami K., Soler D., LaRosa G., Askenase P. W., Williams T. J., Sabroe I. (2000) Basophil responses to chemokines are regulated by both sequential and cooperative receptor signaling. J. Immunol. 165: 7224-7233.

To investigate human basophil responses to chemokines, we have developed a sensitive assay that uses flow cytometry to measure leukocyte shape change as a marker of cell responsiveness. PBMC were isolated from the blood of volunteers. Basophils were identified as a single population of cells that stained positive for IL-3Ralpha (CDw123) and negative for HLA-DR, and their increase in forward scatter (as a result of cell shape change) in response to chemokines was measured. Shape change responses of basophils to chemokines were highly reproducible, with a rank order of potency: monocyte chemoattractant protein (MCP) 4 (peak at/=eotaxin-2=eotaxin-3>/=eotaxin>MCP-1=MCP-3>macrophage-inflammatory protein-1alpha>RANTES=MCP-2=IL-8. The CCR4-selective ligand macrophage-derived chemokine did not elicit a response at concentrations up to 10 nM. Blocking mAbs to CCR2 and CCR3 demonstrated that responses to higher concentrations (>10 nM) of MCP-1 were mediated by CCR3 rather than CCR2, whereas MCP-4 exhibited a biphasic response consistent with sequential activation of CCR3 at lower concentrations and CCR2 at 10 nM MCP-4 and above. In contrast, responses to MCP-3 were blocked only in the presence of both mAbs, but not after pretreatment with either anti-CCR2 or anti-CCR3 mAb alone. These patterns of receptor usage were different from those seen for eosinophils and monocytes. We suggest that cooperation between CCRs might be a mechanism for preferential recruitment of basophils, as occurs in tissue hypersensitivity responses in vivo.

PMID: 11120855

NOV17c

Expression of gene NOV17c, also known as CG56659-02, was assessed using the primer-probe set Ag2970, described in Table NA. Results of the RTQ-PCR runs are shown in Table NB.

TABLE NA
Probe Name Ag2970

| | | | Start | |
|---|---|---|---|---|
| Primers | Sequence | Length | Position | SEQ ID NO: |
| Forward | 5′-acatccatctccacacacctat-3′ | 22 | 90 | 1042 |
| Probe | TET-5′-agtcagctctccctcattgacctaaa-3′-TAMRA | 26 | 125 | 1043 |
| Reverse | 5′-taaaccatctttggaacaatgg-3′ | 22 | 162 | 1044 |

TABLE NB
CNS_neurodegeneration_v1.0

| | Rel. Exp. (%) Ag2970, | | Rel. Exp. (%) Ag2970, |
|---|---|---|---|
| Tissue Name | Run 211008706 | Tissue Name | Run 211008706 |
| AD 1 Hippo | 2.3 | Control (Path) 3 | 2.1 |
| | | Temporal Ctx | |
| AD 2 Hippo | 16.7 | Control (Path) 4 | 38.2 |
| | | Temporal Ctx | |
| AD 3 Hippo | 3.5 | AD 1 Occipital Ctx | 3.7 |
| AD 4 Hippo | 2.2 | AD 2 Occipital Ctx | 0.0 |
| | | (Missing) | |
| AD 5 hippo | 100.0 | AD 3 Occipital Ctx | 4.1 |
| AD 6 Hippo | 28.1 | AD 4 Occipital Ctx | 20.4 |
| Control 2 Hippo | 10.2 | AD 5 Occipital Ctx | 11.2 |
| Control 4 Hippo | 1.9 | AD 6 Occipital Ctx | 18.8 |
| Control (Path) 3 | 2.4 | Control 1 Occipital | 0.0 |
| Hippo | | Ctx | |
| AD 1 Temporal Ctx | 3.4 | Control 2 Occipital | 27.2 |
| | | Ctx | |
| AD 2 Temporal Ctx | 23.0 | Control 3 Occipital | 14.0 |
| | | Ctx | |
| AD 3 Temporal Ctx | 1.0 | Control 4 Occipital | 4.8 |
| | | Ctx | |
| AD 4 Temporal Ctx | 18.2 | Control (Path) 1 | 87.7 |
| | | Occipital Ctx | |
| AD 5 Inf Temporal | 99.3 | Control (Path) 2 | 9.9 |
| Ctx | | Occipital Ctx | |
| AD 5 SupTemporal | 30.1 | Control (Path) 3 | 0.5 |
| Ctx | | Occipital Ctx | |
| AD 6 Inf Temporal | 47.0 | Control (Path) 4 | 13.5 |
| Ctx | | Occipital Ctx | |
| AD 6 Sup Temporal | 59.9 | Control 1 Parietal | 1.1 |
| Ctx | | Ctx | |
| Control 1 Temporal | 0.0 | Control 2 Parietal | 40.1 |
| Ctx | | Ctx | |
| Control 2 Temporal | 17.0 | Control 3 Parietal | 13.2 |
| Ctx | | Ctx | |
| Control 3 Temporal | 10.7 | Control (Path) 1 | 51.1 |
| Ctx | | Parietal Ctx | |
| Control 4 Temporal | 6.9 | Control (Path) 2 | 14.9 |
| Ctx | | Parietal Ctx | |
| Control (Path) 1 | 48.0 | Control (Path) 3 | 0.0 |
| Temporal Ctx | | Parietal Ctx | |
| Control (Path) 2 | 17.6 | Control (Path) 4 | 38.4 |
| Temporal Ctx | | Parietal Ctx | |

CNS_neurodegeneration_v1.0 Summary: Ag2970 The NOV17c gene represents a novel G-protein coupled receptor (GPCR) with expression in the brain. The GPCR family of receptors contains a large number of neurotransmitter receptors, including the dopamine, serotonin, a and b-adrenergic, acetylcholine muscarinic, histamine, peptide, and metabotropic glutamate receptors. GPCRs are excellent drug targets in various neurologic and psychiatric diseases. All antipsychotics have been shown to act at the dopamine D2 receptor; similarly novel antipsychotics also act at the serotonergic receptor, and often the muscarinic and adrenergic receptors as well. While the majority of antidepressants can be classified as selective serotonin reuptake inhibitors, blockade of the 5-HT1A and a2 adrenergic receptors increases the effects of these drugs. The GPCRs are also of use as drug targets in the treatment of stroke. Blockade of the glutamate receptors may decrease the neuronal death resulting from excitotoxicity; further more the purinergic receptors have also been implicated as drug targets in the treatment of cerebral ischemia. The b-adrenergic receptors have been implicated in the treatment of ADHD with Ritalin, while the a-adrenergic receptors have been implicated in memory. Therefore this gene may be of use as a small molecule target for the treatment of any of the described diseases.

In addition, this GPCR is upregulated in the temporal cortex of Alzheimer's disease patients. Therefore, blockade of this receptor may be of use in the treatment of this disease and decrease neuronal death.

References:

El Yacoubi M, Ledent C, Parmentier M, Bertorelli R, Ongini E, Costentin J, Vaugeois J M. Adenosine A2A receptor antagonists are potential antidepressants: evidence based on pharmacology and A2A receptor knockout mice. Br J Pharmacol September 2001;134(1):68-77

1. Adenosine, an ubiquitous neuromodulator, and its analogues have been shown to produce ‘depressant’ effects in animal models believed to be relevant to depressive disorders, while adenosine receptor antagonists have been found to reverse adenosine-mediated ‘depressant’ effect. 2. We have designed studies to assess whether adenosine A2A receptor antagonists, or genetic inactivation of the receptor would be effective in established screening procedures, such as tail suspension and forced swim tests, which are predictive of clinical antidepressant activity. 3. Adenosine A2A receptor knockout mice were found to be less sensitive to ‘depressant’ challenges than their wildtype littermates. Consistently, the adenosine A2A receptor blockers SCH 58261 (1-10 mg kg(−1), i.p.) and KW 6002 (0.1-10 mg kg(−1), p.o.) reduced the total immobility time in the tail suspension test. 4. The efficacy of adenosine A2A receptor antagonists in reducing immobility time in the tail suspension test was confirmed and extended in two groups of mice. Specifically, SCH 58261 (1-10 mg kg(−1)) and ZM 241385 (15-60 mg kg(−1)) were effective in mice previously screened for having high immobility time, while SCH 58261 at 10 mg kg(−1) reduced immobility of mice that were selectively bred for their spontaneous ‘helplessness’ in this assay. 5. Additional experiments were carried out using the forced swim test. SCH 58261 at 10 mg kg(−1) reduced the immobility time by 61%, while KW 6002 decreased the total immobility time at the doses of 1 and 10 mg kg(−1) by 75 and 79%, respectively. 6. Administration of the dopamine D2 receptor antagonist haloperidol (50-200 microg kg(−1) i.p.) prevented the antidepressant-like effects elicited by SCH 58261 (10 mg kg(−1) i.p.) in forced swim test whereas it left unaltered its stimulant motor effects. 7. In conclusion, these data support the hypothesis that A2A receptor antagonists prolong escape-directed behaviour in two screening tests for antidepressants. Altogether the results support the hypothesis that blockade of the adenosine A2A receptor might be an interesting target for the development of effective antidepressant agents.

Blier P. Pharmacology of rapid-onset antidepressant treatment strategies. Clin Psychiatry 2001;62 Suppl 15:12-7

Although selective serotonin reuptake inhibitors (SSRIs) block serotonin (5-HT) reuptake rapidly, their therapeutic action is delayed. The increase in synaptic 5-HT activates feedback mechanisms mediated by 5-HT1A (cell body) and 5-HT1 B (terminal) autoreceptors, which, respectively, reduce the firing in 5-HT neurons and decrease the amount of 5-HT released per action potential resulting in attenuated 5-HT neurotransmission. Long-term treatment desensitizes the inhibitory 5-HT1 autoreceptors, and 5-HT neurotransmission is enhanced. The time course of these events is similar to the delay of clinical action. The addition of pindolol, which blocks 5-HT1A receptors, to SSRI treatment decouples the feedback inhibition of 5-HT neuron firing and accelerates and enhances the antidepressant response. The neuronal circuitry of the 5-HT and norepinephrine (NE) systems and their connections to forebrain areas believed to be involved in depression has been dissected. The firing of 5-HT neurons in the raphe nuclei is driven, at least partly, by alpha1-adrenoceptor-mediated excitatory inputs from NE neurons. Inhibitory alpha2-adrenoceptors on the NE neuroterminals form part of a feedback control mechanism. Mirtazapine, an antagonist at alpha2-adrenoceptors, does not enhance 5-HT neurotransmission directly but disinhibits the NE activation of 5-HT neurons and thereby increases 5-HT neurotransmission by a mechanism that does not require a time-dependent desensitization of receptors. These neurobiological phenomena may underlie the apparently faster onset of action of mirtazapine compared with the SSRIs.

Tranquillini M E, Reggiani A. Glycine-site antagonists and stroke. Expert Opin Investig Drugs November 1999;8(11):1837-1848

The excitatory amino acid, (S)-glutamic acid, plays an important role in controlling many neuronal processes. Its action is mediated by two main groups of receptors: the ionotropic receptors (which include NMDA, AMPA and kainic acid subtypes) and the metabotropic receptors (mGluR(1-8)) mediating G-protein coupled responses. This review focuses on the strychnine insensitive glycine binding site located on the NMDA receptor channel, and on the possible use of selective antagonists for the treatment of stroke. Stroke is a devastating disease caused by a sudden vascular accident. Neurochemically, a massive release of glutamate occurs in neuronal tissue; this overactivates the NMDA receptor, leading to increased intracellular calcium influx, which causes neuronal cell death through necrosis. NMDA receptor activation strongly depends upon the presence of glycine as a co-agonist. Therefore, the administration of a glycine antagonist can block overactivation of NMDA receptors, thus preserving neurones from damage. The glycine antagonists currently identified can be divided into five main categories depending on their chemical structure: indoles, tetrahydroquinolines, benzoazepines, quinoxalinediones and pyrida-zinoquinolines.

Monopoli A, Lozza G, Forlani A, Mattavelli A, Ongini E. Blockade of adenosine A2A receptors by SCH 58261 results in neuroprotective effects in cerebral ischaemia in rats. Neuroreport Dec. 1, 1998;9(17):3955-9

Blockade of adenosine receptors can reduce cerebral infarct size in the model of global ischaemia. Using the potent and selective A2A adenosine receptor antagonist, SCH 58261, we assessed whether A2A receptors are involved in the neuronal damage following focal cerebral ischaemia as induced by occluding the left middle cerebral artery. SCH 58261 (0.01 mg/kg either i.p. or i.v.) administered to normotensive rats 10 min after ischaemia markedly reduced cortical infarct volume as measured 24 h later (30% vs controls, p<0.05). Similar effects were observed when SCH 58261 (0.01 mg/kg, i.p.) was administered to hypertensive rats (28% infarct volume reduction vs controls, p<0.05). Neuroprotective properties of SCH 58261 administered after ischaemia indicate that blockade of A2A adenosine receptors is a potentially useful biological target for the reduction of brain injury.

Panel 1.3D Summary: Ag2970 Expression of the NOV17c gene is low/undetectable in all samples on this panel (CTs>35). (Data not shown.)

Panel 4D Summary: Ag2970 Expression of the NOV17c gene is low/undetectable in all samples on this panel (CTs>35). (Data not shown.)

NOV19a

Expression of gene NOV19a was assessed using the primer-probe set Ag2972, described in Table OA. Results of the RTQ-PCR runs are shown in Tables OB, OC, and OD.

TABLE OA
Probe Name Ag2972

| | | | Start | |
|---|---|---|---|---|
| Primers | Sequence | Length | Position | SEQ ID NO: |
| Forward | 5′-atgtgtttcagcttccattctg-3′ | 22 | 543 | 1045 |
| Probe | TET-5′-taggtctcggctcattaaccactttt-3′-TAMRA | 26 | 565 | 1046 |
| Reverse | 5′-tgtcctgacacaccaatgatag-3′ | 22 | 611 | 1047 |

TABLE OB
CNS_neurodegeneration_v1.0

| | Rel. Exp. (%) Ag2972, | | Rel. Exp. (%) Ag2972, |
|---|---|---|---|
| Tissue Name | Run 211008969 | Tissue Name | Run 211008969 |
| AD 1 Hippo | 2.1 | Control (Path) 3 | 1.9 |
| | | Temporal Ctx | |
| AD 2 Hippo | 13.7 | Control (Path) 4 | 70.2 |
| | | Temporal Ctx | |
| AD 3 Hippo | 2.2 | AD 1 Occipital Ctx | 3.3 |
| AD 4 Hippo | 1.4 | AD 2 Occipital Ctx | 0.0 |
| | | (Missing) | |
| AD 5 Hippo | 75.8 | AD 3 Occipital Ctx | 2.2 |
| AD 6 Hippo | 33.4 | AD 4 Occipital Ctx | 46.3 |
| Control 2 Hippo | 11.9 | AD 5 Occipital Ctx | 22.5 |
| Control 4 Hippo | 2.5 | AD 6 Occipital Ctx | 19.8 |
| Control (Path) 3 | 1.3 | Control 1 Occipital | 1.1 |
| Hippo | | Ctx | |
| AD 1 Temporal Ctx | 2.1 | Control 2 Occipital | 42.0 |
| | | Ctx | |
| AD 2 Temporal Ctx | 28.3 | Control 3 Occipital | 21.9 |
| | | Ctx | |
| AD 3 Temporal Ctx | 1.4 | Control 4 Occipital | 10.4 |
| | | Ctx | |
| AD 4 Temporal Ctx | 44.8 | Control (Path) 1 | 100.0 |
| | | Occipital Ctx | |
| AD 5 Inf Temporal | 78.5 | Control (Path) 2 | 3.5 |
| Ctx | | Occipital Ctx | |
| AD 5 Sup Temporal | 12.0 | Control (Path) 3 | 1.1 |
| Ctx | | Occipital Ctx | |
| AD 6 Inf Temporal | 33.7 | Control (Path) 4 | 26.6 |
| Ctx | | Occipital Ctx | |
| AD 6 Sup Temporal | 56.3 | Control 1 Parietal | 0.6 |
| Ctx | | Ctx | |
| Control 1 Temporal | 0.0 | Control 2 Parietal | 18.8 |
| Ctx | | Ctx | |
| Control 2 Temporal | 10.4 | Control 3 Parietal | 17.7 |
| Ctx | | Ctx | |
| Control 3 Temporal | 11.5 | Control (Path) 1 | 77.4 |
| Ctx | | Parietal Ctx | |
| Control 3 Temporal | 7.8 | Control (Path) 2 | 10.3 |
| Ctx | | Parietal Ctx | |
| Control (Path) 1 | 52.9 | Control (Path) 3 | 0.7 |
| Temporal Ctx | | Parietal Ctx | |
| Control (Path) 2 | 7.9 | Control (Path) 4 | 57.8 |
| Temporal Ctx | | Parietal Ctx | |

TABLE OC
Panel 4D

| | Rel. Exp. (%) | | Rel. Exp. (%) |
|---|---|---|---|
| | Ag2972, Run | | Ag2972, Run |
| Tissue Name | 164314417 | Tissue Name | 164314417 |
| Secondary Th1 act | 0.0 | HUVEC IL-1beta | 0.0 |
| Secondary Th2 act | 0.0 | HUVEC IFN gamma | 11.3 |
| Secondary Tr1 act | 0.0 | HUVEC TNF alpha + IFN | 0.0 |
| | | gamma | |
| Secondary Th1 rest | 4.1 | HUVEC TNF alpha + IL4 | 0.0 |
| Secondary Th2 rest | 2.1 | HUVEC IL-11 | 0.0 |
| Secondary Tr1 rest | 0.0 | Lung Microvascular EC | 0.0 |
| | | none | |
| Primary Th1 act | 12.2 | Lung Microvascular EC | 4.2 |
| | | TNF alpha + IL-1beta | |
| Primary Th2 act | 0.0 | Microvascular Dermal EC | 0.0 |
| | | none | |
| Primary Tr1 act | 0.0 | Microsvasular Dermal EC | 0.0 |
| | | TNF alpha + IL-1beta | |
| Primary Th1 rest | 37.4 | Bronchial epithelium | 7.6 |
| | | TNF alpha + IL1beta | |
| Primary Th2 rest | 46.7 | Small airway epithelium | 0.0 |
| | | none | |
| Primary Tr1 rest | 9.7 | Small airway epithelium | 0.0 |
| | | TNF alpha + IL-1beta | |
| CD45RA CD4 | 21.2 | Coronery artery SMC rest | 22.2 |
| lymphocyte act | | | |
| CD45RO CD4 | 16.3 | Coronery artery SMC | 0.0 |
| lymphocyte act | | TNF alpha + IL-1beta | |
| CD8 lymphocyte act | 8.4 | Astrocytes rest | 0.0 |
| Secondary CD8 | 11.6 | Astrocytes TNF alpha + | 0.0 |
| lymphocyte rest | | IL-1beta | |
| Secondary CD8 | 0.0 | KU-812 (Basophil) rest | 20.3 |
| lymphocyte act | | | |
| CD4 lymphocyte none | 25.5 | KU-812 (Basophil) | 34.6 |
| | | PMA/ionomycin | |
| 2ry Th1/Th2/Tr1_anti- | 0.0 | CCD1106 (Keratinocytes) | 0.0 |
| CD95 CH11 | | none | |
| LAK cells rest | 26.1 | CCD1106 (Keratinocytes) | 8.5 |
| | | TNF alpha + IL-1beta | |
| LAK cells IL-2 | 46.7 | Liver cirrhosis | 22.8 |
| LAK cells IL-2 + IL-12 | 32.5 | Lupus kidney | 0.0 |
| LAK cells IL-2 + IFN | 49.0 | NCI-H292 none | 0.0 |
| gamma | | | |
| LAK cells IL-2 + IL-18 | 38.2 | NCI-H292 IL-4 | 0.0 |
| LAK cells | 5.7 | NCI-H292 IL-9 | 0.0 |
| PMA/ionomycin | | | |
| NK Cells IL-2 rest | 7.7 | NCI-H292 IL-13 | 0.0 |
| Two Way MLR 3 day | 73.2 | NCI-H292 IFN gamma | 0.0 |
| Two Way MLR 5 day | 24.8 | HPAEC none | 0.0 |
| Two Way MLR 7 day | 0.0 | HPAEC TNF alpha + IL- | 0.0 |
| | | 1beta | |
| PBMC rest | 1.7 | Lung fibroblast none | 0.0 |
| PBMC PWM | 100.0 | Lung fibroblast TNF alpha + | 0.0 |
| | | IL-1beta | |
| PBMC PHA-L | 41.2 | Lung fibroblast IL-4 | 0.0 |
| Ramos (B cell) none | 0.0 | Lung fibroblast IL-9 | 0.0 |
| Ramos (B cell) | 10.0 | Lung fibroblast IL-13 | 0.0 |
| ionomycin | | | |
| B lymphocytes PWM | 40.1 | Lung fibroblast IFN | 0.0 |
| | | gamma | |
| B lymphocytes CD40L | 21.6 | Dermal fibroblast | 0.0 |
| and IL-4 | | CCD1070 rest | |
| EOL-1 dbcAMP | 0.0 | Dermal fibroblast | 6.6 |
| | | CCD1070 TNF alpha | |
| EOL-1 dbcAMP | 0.0 | Dermal fibroblast | 0.0 |
| PMA/ionomycin | | CCD1070 IL-1beta | |
| Dendritic cells none | 21.9 | Dermal fibroblast IFN | 0.0 |
| | | gamma | |
| Dendritic cells LPS | 0.0 | Dermal fibroblast IL-4 | 0.0 |
| Dendritic cells anti- | 0.0 | IBD Colitis 2 | 2.9 |
| CD40 | | | |
| Monocytes rest | 0.0 | IBD Crohn's | 0.0 |
| Monocytes LPS | 9.2 | Colon | 7.6 |
| Macrophages rest | 19.6 | Lung | 0.0 |
| Macrophages LPS | 8.1 | Thymus | 11.8 |
| HUVEC none | 0.0 | Kidney | 71.7 |
| HUVEC starved | 0.0 | | |

TABLE OD
Panel CNS_1

| | Rel. Exp. (%) Ag2972, | | Rel. Exp. (%) Ag2972, |
|---|---|---|---|
| Tissue Name | Run 171670020 | Tissue Name | Run 171670020 |
| BA4 Control | 17.8 | BA17 PSP | 52.5 |
| BA4 Control2 | 8.6 | BA17 PSP2 | 3.1 |
| BA4 | 7.1 | Sub Nigra Control | 31.0 |
| Alzheimer's2 | | | |
| BA4 Parkinson's | 42.9 | Sub Nigra Control2 | 14.7 |
| BA4 | 51.1 | Sub Nigra | 5.1 |
| Parkinson's2 | | Alzheimer's2 | |
| BA4 | 7.4 | Sub Nigra | 28.9 |
| Huntington's | | Parkinson's2 | |
| BA4 | 0.0 | Sub Nigra | 100.0 |
| Huntington's2 | | Huntington's | |
| BA4 PSP | 0.0 | Sub Nigra | 11.2 |
| | | Huntington's2 | |
| BA4 PSP2 | 3.7 | Sub Nigra PSP2 | 1.3 |
| BA4 Depression | 16.7 | Sub Nigra | 7.4 |
| | | Depression | |
| BA4 | 0.0 | Sub Nigra | 2.5 |
| Depression2 | | Depression2 | |
| BA7 Control | 15.7 | Glob Palladus | 7.8 |
| | | Control | |
| BA7 Control2 | 15.7 | Glob Palladus | 0.0 |
| | | Control2 | |
| BA7 | 0.0 | Glob Palladus | 0.0 |
| Alzheimer's2 | | Alzheimer's | |
| BA7 Parkinson's | 24.3 | Glob Palladus | 2.0 |
| | | Alzheimer's2 | |
| BA7 | 29.9 | Glob Palladus | 57.0 |
| Parkinson's2 | | Parkinson's | |
| BA7 | 43.5 | Glob Palladus | 3.8 |
| Huntington's | | Parkinson's2 | |
| BA7 | 7.2 | Glob Palladus PSP | 0.0 |
| Huntington's2 | | | |
| BA7 PSP | 20.2 | Glob Palladus PSP2 | 3.4 |
| BA7 PSP2 | 2.1 | Glob Palladus | 19.5 |
| | | Depression | |
| BA7 Depression | 5.2 | Temp Pole Control | 0.0 |
| BA9 Control | 0.0 | Temp Pole Control2 | 3.3 |
| BA9 Control2 | 22.7 | Temp Pole | 0.0 |
| | | Alzheimer's | |
| BA9 Alzheimer's | 1.3 | Temp Pole | 8.6 |
| | | Alzheimer's2 | |
| BA9 | 9.0 | Temp Pole | 0.0 |
| Alzheimer's2 | | Parkinson's | |
| BA9 Parkinson's | 11.7 | Temp Pole | 23.0 |
| | | Parkinson's2 | |
| BA9 | 23.7 | Temp Pole | 7.2 |
| Parkinson's2 | | Huntington's | |
| BA9 | 12.4 | Temp Pole PSP | 5.4 |
| Huntington's | | | |
| BA9 | 7.0 | Temp Pole PSP2 | 3.6 |
| Huntington's2 | | | |
| BA9 PSP | 13.6 | Temp Pole | 5.9 |
| | | Depression2 | |
| BA9 PSP2 | 5.6 | Cing Gyr Control | 51.4 |
| BA9 Depression | 12.6 | Cing Gyr Control2 | 0.0 |
| BA9 | 2.7 | Cing Gyr | 6.7 |
| Depression2 | | Alzheimer's | |
| BA17 Control | 75.8 | Cing Gyr | 2.7 |
| | | Alzheimer's2 | |
| BA17 Control2 | 15.8 | Cing Gyr Parkinson's | 38.4 |
| BA17 | 8.4 | Cing Gyr | 11.6 |
| Alzheimer's2 | | Parkinson's2 | |
| BA17 | 32.3 | Cing Gyr | 60.3 |
| Parkinson's | | Huntington's | |
| BA17 | 51.4 | Cing Gyr | 0.0 |
| Parkinson's2 | | Huntington's2 | |
| BA17 | 22.4 | Cing Gyr PSP | 23.7 |
| Huntington's | | | |
| BA17 | 5.2 | Cing Gyr PSP2 | 0.0 |
| Huntington's2 | | | |
| BA17 | 17.6 | Cing Gyr Depression | 8.5 |
| Depression | | | |
| BA17 | 16.6 | Cing Gyr | 8.1 |
| Depression2 | | Depression2 | |

CNS_neurodegeneration_v1.0 Summary: Ag2972 The NOV19a represents a novel G-protein coupled receptor (GPCR) with expression in the brain. The GPCR family of receptors contains a large number of neurotransmitter receptors, including the dopamine, serotonin, a and b-adrenergic, acetylcholine muscarinic, histamine, peptide, and metabotropic glutamate receptors. GPCRs are excellent drug targets in various neurologic and psychiatric diseases. All antipsychotics have been shown to act at the dopamine D2 receptor; similarly novel antipsychotics also act at the serotonergic receptor, and often the muscarinic and adrenergic receptors as well. While the majority of antidepressants can be classified as selective serotonin reuptake inhibitors, blockade of the 5-HT1A and a2 adrenergic receptors increases the effects of these drugs. The GPCRs are also of use as drug targets in the treatment of stroke. Blockade of the glutamate receptors may decrease the neuronal death resulting from excitotoxicity; further more the purinergic receptors have also been implicated as drug targets in the treatment of cerebral ischemia. The b-adrenergic receptors have been implicated in the treatment of ADHD with Ritalin, while the a-adrenergic receptors have been implicated in memory. Therefore this gene may be of use as a small molecule target for the treatment of any of the described diseases.

References:

El Yacoubi M, Ledent C, Parmentier M, Bertorelli R, Ongini E, Costentin J, Vaugeois J M. Adenosine A2A receptor antagonists are potential antidepressants: evidence based on pharmacology and A2A receptor knockout mice. Br J Pharmacol September 2001;134(1):68-77

1. Adenosine, an ubiquitous neuromodulator, and its analogues have been shown to produce ‘depressant’ effects in animal models believed to be relevant to depressive disorders, while adenosine receptor antagonists have been found to reverse adenosine-mediated ‘depressant’ effect. 2. We have designed studies to assess whether adenosine A2A receptor antagonists, or genetic inactivation of the receptor would be effective in established screening procedures, such as tail suspension and forced swim tests, which are predictive of clinical antidepressant activity. 3. Adenosine A2A receptor knockout mice were found to be less sensitive to ‘depressant’ challenges than their wildtype littermates. Consistently, the adenosine A2A receptor blockers SCH 58261 (1-10 mg kg(−1), i.p.) and KW 6002 (0.1-10 mg kg(−1), p.o.) reduced the total immobility time in the tail suspension test. 4. The efficacy of adenosine A2A receptor antagonists in reducing immobility time in the tail suspension test was confirmed and extended in two groups of mice. Specifically, SCH 58261 (1-10 mg kg(−1)) and ZM 241385 (15-60 mg kg(−1)) were effective in mice previously screened for having high immobility time, while SCH 58261 at 10 mg kg(−1) reduced immobility of mice that were selectively bred for their spontaneous ‘helplessness’ in this assay. 5. Additional experiments were carried out using the forced swim test. SCH 58261 at 10 mg kg(−1) reduced the immobility time by 61%, while KW 6002 decreased the total immobility time at the doses of 1 and 10 mg kg(−1) by 75 and 79%, respectively. 6. Administration of the dopamine D2 receptor antagonist haloperidol (50-200 microg kg(−1) i.p.) prevented the antidepressant-like effects elicited by SCH 58261 (10 mg kg(−1) i.p.) in forced swim test whereas it left unaltered its stimulant motor effects. 7. In conclusion, these data support the hypothesis that A2A receptor antagonists prolong escape-directed behaviour in two screening tests for antidepressants. Altogether the results support the hypothesis that blockade of the adenosine A2A receptor might be an interesting target for the development of effective antidepressant agents.

Blier P. Pharmacology of rapid-onset antidepressant treatment strategies. Clin Psychiatry 2001;62 Suppl 15:12-7

Although selective serotonin reuptake inhibitors (SSRIs) block serotonin (5-HT) reuptake rapidly, their therapeutic action is delayed. The increase in synaptic 5-HT activates feedback mechanisms mediated by 5-HT1A (cell body) and 5-HT1 B (terminal) autoreceptors, which, respectively, reduce the firing in 5-HT neurons and decrease the amount of 5-HT released per action potential resulting in attenuated 5-HT neurotransmission. Long-term treatment desensitizes the inhibitory 5-HT1 autoreceptors, and 5-HT neurotransmission is enhanced. The time course of these events is similar to the delay of clinical action. The addition of pindolol, which blocks 5-HT1A receptors, to SSRI treatment decouples the feedback inhibition of 5-HT neuron firing and accelerates and enhances the antidepressant response. The neuronal circuitry of the 5-HT and norepinephrine (NE) systems and their connections to forebrain areas believed to be involved in depression has been dissected. The firing of 5-HT neurons in the raphe nuclei is driven, at least partly, by alpha1-adrenoceptor-mediated excitatory inputs from NE neurons. Inhibitory alpha2-adrenoceptors on the NE neuroterminals form part of a feedback control mechanism. Mirtazapine, an antagonist at alpha2-adrenoceptors, does not enhance 5-HT neurotransmission directly but disinhibits the NE activation of 5-HT neurons and thereby increases 5-HT neurotransmission by a mechanism that does not require a time-dependent desensitization of receptors. These neurobiological phenomena may underlie the apparently faster onset of action of mirtazapine compared with the SSRIs.

Tranquillini M E, Reggiani A. Glycine-site antagonists and stroke. Expert Opin Investig Drugs November 1999;8(11):1837-1848

The excitatory amino acid, (S)-glutamic acid, plays an important role in controlling many neuronal processes. Its action is mediated by two main groups of receptors: the ionotropic receptors (which include NMDA, AMPA and kainic acid subtypes) and the metabotropic receptors (mGluR(1-8)) mediating G-protein coupled responses. This review focuses on the strychnine insensitive glycine binding site located on the NMDA receptor channel, and on the possible use of selective antagonists for the treatment of stroke. Stroke is a devastating disease caused by a sudden vascular accident. Neurochemically, a massive release of glutamate occurs in neuronal tissue; this overactivates the NMDA receptor, leading to increased intracellular calcium influx, which causes neuronal cell death through necrosis. NMDA receptor activation strongly depends upon the presence of glycine as a co-agonist. Therefore, the administration of a glycine antagonist can block overactivation of NMDA receptors, thus preserving neurones from damage. The glycine antagonists currently identified can be divided into five main categories depending on their chemical structure: indoles, tetrahydroquinolines, benzoazepines, quinoxalinediones and pyrida-zinoquinolines.

Monopoli A, Lozza G, Forlani A, Mattavelli A, Ongini E. Blockade of adenosine A2A receptors by SCH 58261 results in neuroprotective effects in cerebral ischaemia in rats. Neuroreport Dec. 1, 1998;9(17):3955-9

Blockade of adenosine receptors can reduce cerebral infarct size in the model of global ischaemia. Using the potent and selective A2A adenosine receptor antagonist, SCH 58261, we assessed whether A2A receptors are involved in the neuronal damage following focal cerebral ischaemia as induced by occluding the left middle cerebral artery. SCH 58261 (0.01 mg/kg either i.p. or i.v.) administered to normotensive rats 10 min after ischaemia markedly reduced cortical infarct volume as measured 24 h later (30% vs controls, p<0.05). Similar effects were observed when SCH 58261 (0.01 mg/kg, i.p.) was administered to hypertensive rats (28% infarct volume reduction vs controls, p<0.05). Neuroprotective properties of SCH 58261 administered after ischaemia indicate that blockade of A2A adenosine receptors is a potentially useful biological target for the reduction of brain injury.

Panel 1.3D Summary: Ag2972 Expression of the NOV19a gene is low/undetectable in all samples on this panel (CTs>35). (Data not shown.)

Panel 4D Summary: Ag2972 Expression of the the NOV19a gene is restricted to a few samples in this panel, with highest expression in peripheral blood mononuclear cells (PBMC) treated with the B cell selective pokeweed mitogen. No significant levels of expression of the transcript are seen in PBMC that contain normal B cells. Therefore, the putative GPCR encoded by this gene could potentially be used diagnostically to identify activated B cells. In addition, the gene product could also potentially be used therapeutically in the treatment of diseases in which B cells are activated.

Panel CNS_{—}1 Summary: Ag2972 This panel confirms expression of the NOV19a gene in the brains of an independent group of subjects. Please see panel 1.3d for a discussion of utility of this gene in the central nervous system.

NOV20

Expression of gene NOV20 was assessed using the primer-probe set Ag2973, described in Table PA. Results of the RTQ-PCR runs are shown in Tables PB and PC.

TABLE PA
Probe Name Ag2973

| | | | Start | |
|---|---|---|---|---|
| Primers | Sequence | Length | Position | SEQ ID NO: |
| Forward | 5′-gctgtgtggctcaagtctattt-3′ | 22 | 287 | 1048 |
| Probe | TET-5′-ttctctgcctttgcatctgctgagct-3′-TAMRA | 26 | 310 | 1049 |
| Reverse | 5′-agcggtcataagacatgacagt-3′ | 22 | 346 | 1050 |

TABLE PB
CNS_neurodegeneration_v1.0

| | Rel. Exp. (%) Ag2973, | | Rel. Exp. (%) Ag2973, |
|---|---|---|---|
| Tissue Name | Run 209779096 | Tissue Name | Run 209779096 |
| AD 1 Hippo | 4.9 | Control (Path) 3 | 1.7 |
| | | Temporal Ctx | |
| AD 2 Hippo | 16.2 | Control (Path) 4 | 46.7 |
| | | Temporal Ctx | |
| AD 3 Hippo | 2.2 | AD 1 Occipital Ctx | 3.9 |
| AD 4 Hippo | 3.7 | AD 2 Occipital Ctx | 0.0 |
| | | (Missing) | |
| AD 5 Hippo | 50.7 | AD 3 Occipital Ctx | 1.7 |
| AD 6 Hippo | 16.4 | AD 4 Occipital Ctx | 19.1 |
| Control 2 Hippo | 8.2 | AD 5 Occipital Ctx | 24.5 |
| Control 4 Hippo | 0.5 | AD 6 Occipital Ctx | 2.7 |
| Control (Path) 3 | 0.0 | Control 1 Occipital | 0.0 |
| Hippo | | Ctx | |
| AD 1 Temporal Ctx | 4.5 | Control 2 Occipital | 17.8 |
| | | Ctx | |
| AD 2 Temporal Ctx | 27.7 | Control 3 Occipital | 8.7 |
| | | Ctx | |
| AD 3 Temporal Ctx | 3.6 | Control 4 Occipital | 0.7 |
| | | Ctx | |
| AD 4 Temporal Ctx | 17.6 | Control (Path) 1 | 74.7 |
| | | Occipital Ctx | |
| AD 5 Inf Temporal | 64.2 | Control (Path) 2 | 13.2 |
| Ctx | | Occipital Ctx | |
| AD 5 Sup Temporal | 21.8 | Control (Path) 3 | 0.0 |
| Ctx | | Occipital Ctx | |
| AD 6 Inf Temporal | 24.3 | Control (Path) 4 | 9.2 |
| Ctx | | Occipital Ctx | |
| AD 6 Sup Temporal | 22.2 | Control 1 Parietal | 1.5 |
| Ctx | | Ctx | |
| Control 1 Temporal | 1.4 | Control 2 Parietal | 27.9 |
| Ctx | | Ctx | |
| Control 2 Temporal | 33.9 | Control 3 Parietal | 16.8 |
| Ctx | | Ctx | |
| Control 3 Temporal | 18.4 | Control (Path) 1 | 95.3 |
| Ctx | | Parietal Ctx | |
| Control 3 Temporal | 3.2 | Control (Path) 2 | 23.7 |
| Ctx | | Parietal Ctx | |
| Control (Path) 1 | 100.0 | Control (Path) 3 | 1.4 |
| Temporal Ctx | | Parietal Ctx | |
| Control (Path) 2 | 68.8 | Control (Path) 4 | 33.7 |
| Temporal Ctx | | Parietal Ctx | |

TABLE PC
Panel 4D

| | Rel. Exp. (%) | | Rel. Exp. (%) |
|---|---|---|---|
| | Ag2973, Run | | Ag2973, Run |
| Tissue Name | 164329850 | Tissue Name | 164329850 |
| Secondary Th1 act | 0.0 | HUVEC IL-1beta | 0.0 |
| Secondary Th2 act | 0.0 | HUVEC IFN gamma | 0.0 |
| Secondary Tr1 act | 0.0 | HUVEC TNF alpha + IFN | 0.0 |
| | | gamma | |
| Secondary Th1 rest | 11.7 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC | 0.0 |
| | | none | |
| Primary Th1 act | 0.0 | Lung Microvascular EC | 0.0 |
| | | TNF alpha + IL-1beta | |
| Primary Th2 act | 0.0 | Microvascular Dermal EC | 0.0 |
| | | none | |
| Primary Tr1 act | 0.0 | Microsvasular Dermal EC | 0.0 |
| | | TNF alpha + IL-1beta | |
| Primary Th1 rest | 0.0 | Bronchial epithelium | 0.0 |
| | | TNF alpha + IL1beta | |
| Primary Th2 rest | 6.0 | Small airway epithelium | 0.0 |
| | | none | |
| Primary Tr1 rest | 0.0 | Small airway epithelium | 0.0 |
| | | TNF alpha + IL-1beta | |
| CD45RA CD4 | 0.0 | Coronery artery SMC rest | 0.0 |
| lymphocyte act | | | |
| CD45RO CD4 | 0.0 | Coronery artery SMC | 0.0 |
| lymphocyte act | | TNF alpha + IL-1beta | |
| CD8 lymphocyte act | 0.0 | Astrocytes rest | 0.0 |
| Secondary CD8 | 0.0 | Astrocytes TNF alpha + | 0.0 |
| lymphocyte rest | | IL-1beta | |
| Secondary CD8 | 0.0 | KU-812 (Basophil) rest | 0.0 |
| lymphocyte act | | | |
| CD4 lymphocyte none | 0.0 | KU-812 (Basophil) | 0.0 |
| | | PMA/ionomycin | |
| 2ry Th1/Th2/Tr1_anti- | 0.0 | CCD1106 (Keratinocytes) | 0.0 |
| CD95 CH11 | | none | |
| LAK cells rest | 0.0 | CCD1106 (Keratinocytes) | 0.0 |
| | | TNF alpha + IL-1beta | |
| LAK cells IL-2 | 0.0 | Liver cirrhosis | 100.0 |
| LAK cells IL-2 + IL-12 | 0.0 | Lupus kidney | 9.7 |
| LAK cells IL-2 + IFN | 0.0 | NCI-H292 none | 0.0 |
| gamma | | | |
| LAK cells IL-2 + IL-18 | 0.0 | NCI-H292 IL-4 | 0.0 |
| LAK cells | 0.0 | NCI-H292 IL-9 | 0.0 |
| PMA/ionomycin | | | |
| NK Cells IL-2 rest | 0.0 | NCI-H292 IL-13 | 0.0 |
| Two Way MLR 3 day | 0.0 | NCI-H292 IFN gamma | 0.0 |
| Two Way MLR 5 day | 0.0 | HPAEC none | 0.0 |
| Two Way MLR 7 day | 0.0 | HPAEC TNF alpha + IL- | 0.0 |
| | | 1beta | |
| PBMC rest | 0.0 | Lung fibroblast none | 0.0 |
| PBMC PWM | 0.0 | Lung fibroblast TNF alpha + | 0.0 |
| | | IL-1beta | |
| PBMC PHA-L | 0.0 | Lung fibroblast IL-4 | 0.0 |
| Ramos (B cell) none | 0.0 | Lung fibroblast IL-9 | 0.0 |
| Ramos (B cell) | 0.0 | Lung fibroblast IL-13 | 0.0 |
| ionomycin | | | |
| B lymphocytes PWM | 0.0 | Lung fibroblast IFN | 0.0 |
| | | gamma | |
| B lymphocytes CD40L | 0.0 | Dermal fibroblast | 0.0 |
| and IL-4 | | CCD1070 rest | |
| EOL-1 dbcAMP | 0.0 | Dermal fibroblast | 0.0 |
| | | CCD1070 TNF alpha | |
| EOL-1 dbcAMP | 0.0 | Dermal fibroblast | 0.0 |
| PMA/ionomycin | | CCD1070 IL-1beta | |
| Dendritic cells none | 0.0 | Dermal fibroblast IFN | 0.0 |
| | | gamma | |
| Dendritic cells LPS | 0.0 | Dermal fibroblast IL-4 | 0.0 |
| Dendritic cells anti- | 0.0 | IBD Colitis 2 | 0.0 |
| CD40 | | | |
| Monocytes rest | 0.0 | IBD Crohn's | 0.0 |
| Monocytes LPS | 0.0 | Colon | 8.7 |
| Macrophages rest | 0.0 | Lung | 6.6 |
| Macrophages LPS | 0.0 | Thymus | 13.9 |
| HUVEC none | 0.0 | Kidney | 0.0 |
| HUVEC starved | 0.0 | | |

CNS_neurodegeneration_v1.0 Summary: Ag2973 The NOV20 gene represents a novel G-protein coupled receptor (GPCR) with expression in the brain. The GPCR family of receptors contains a large number of neurotransmitter receptors, including the dopamine, serotonin, a and b-adrenergic, acetylcholine muscarinic, histamine, peptide, and metabotropic glutamate receptors. GPCRs are excellent drug targets in various neurologic and psychiatric diseases. All antipsychotics have been shown to act at the dopamine D2 receptor; similarly novel antipsychotics also act at the serotonergic receptor, and often the muscarinic and adrenergic receptors as well. While the majority of antidepressants can be classified as selective serotonin reuptake inhibitors, blockade of the 5-HT1A and a2 adrenergic receptors increases the effects of these drugs. The GPCRs are also of use as drug targets in the treatment of stroke. Blockade of the glutamate receptors may decrease the neuronal death resulting from excitotoxicity; further more the purinergic receptors have also been implicated as drug targets in the treatment of cerebral ischemia. The b-adrenergic receptors have been implicated in the treatment of ADHD with Ritalin, while the a-adrenergic receptors have been implicated in memory. Therefore this gene may be of use as a small molecule target for the treatment of any of the described diseases.

References:

El Yacoubi M, Ledent C, Parmentier M, Bertorelli R, Ongini E, Costentin J, Vaugeois J M. Adenosine A2A receptor antagonists are potential antidepressants: evidence based on pharmacology and A2A receptor knockout mice. Br J Pharmacol September 2001;134(1):68-77

1. Adenosine, an ubiquitous neuromodulator, and its analogues have been shown to produce ‘depressant’ effects in animal models believed to be relevant to depressive disorders, while adenosine receptor antagonists have been found to reverse adenosine-mediated ‘depressant’ effect. 2. We have designed studies to assess whether adenosine A2A receptor antagonists, or genetic inactivation of the receptor would be effective in established screening procedures, such as tail suspension and forced swim tests, which are predictive of clinical antidepressant activity. 3. Adenosine A2A receptor knockout mice were found to be less sensitive to ‘depressant’ challenges than their wildtype littermates. Consistently, the adenosine A2A receptor blockers SCH 58261 (1-10 mg kg(−1), i.p.) and KW 6002 (0.1-10 mg kg(−1), p.o.) reduced the total immobility time in the tail suspension test. 4. The efficacy of adenosine A2A receptor antagonists in reducing immobility time in the tail suspension test was confirmed and extended in two groups of mice. Specifically, SCH 58261 (1-10 mg kg(−1)) and ZM 241385 (15-60 mg kg(−1)) were effective in mice previously screened for having high immobility time, while SCH 58261 at 10 mg kg(−1) reduced immobility of mice that were selectively bred for their spontaneous ‘helplessness’ in this assay. 5. Additional experiments were carried out using the forced swim test. SCH 58261 at 10 mg kg(−1) reduced the immobility time by 61%, while KW 6002 decreased the total immobility time at the doses of 1 and 10 mg kg(−1) by 75 and 79%, respectively. 6.Administration of the dopamine D2 receptor antagonist haloperidol (50-200 microg kg(−1) i.p.) prevented the antidepressant-like effects elicited by SCH 58261 (10 mg kg(−1) i.p.) in forced swim test whereas it left unaltered its stimulant motor effects. 7. In conclusion, these data support the hypothesis that A2A receptor antagonists prolong escape-directed behaviour in two screening tests for antidepressants. Altogether the results support the hypothesis that blockade of the adenosine A2A receptor might be an interesting target for the development of effective antidepressant agents.

Blier P. Pharmacology of rapid-onset antidepressant treatment strategies. Clin Psychiatry 2001;62 Suppl 15:12-7

Although selective serotonin reuptake inhibitors (SSRIs) block serotonin (5-HT) reuptake rapidly, their therapeutic action is delayed. The increase in synaptic 5-HT activates feedback mechanisms mediated by 5-HT1A (cell body) and 5-HT1 B (terminal) autoreceptors, which, respectively, reduce the firing in 5-HT neurons and decrease the amount of 5-HT released per action potential resulting in attenuated 5-HT neurotransmission. Long-term treatment desensitizes the inhibitory 5-HT1 autoreceptors, and 5-HT neurotransmission is enhanced. The time course of these events is similar to the delay of clinical action. The addition of pindolol, which blocks 5-HT1A receptors, to SSRI treatment decouples the feedback inhibition of 5-HT neuron firing and accelerates and enhances the antidepressant response. The neuronal circuitry of the 5-HT and norepinephrine (NE) systems and their connections to forebrain areas believed to be involved in depression has been dissected. The firing of 5-HT neurons in the raphe nuclei is driven, at least partly, by alpha1-adrenoceptor-mediated excitatory inputs from NE neurons. Inhibitory alpha2-adrenoceptors on the NE neuroterminals form part of a feedback control mechanism. Mirtazapine, an antagonist at alpha2-adrenoceptors, does not enhance 5-HT neurotransmission directly but disinhibits the NE activation of 5-HT neurons and thereby increases 5-HT neurotransmission by a mechanism that does not require a time-dependent desensitization of receptors. These neurobiological phenomena may underlie the apparently faster onset of action of mirtazapine compared with the SSRIs.

Tranquillini M E, Reggiani A. Glycine-site antagonists and stroke. Expert Opin Investig Drugs November 1999;8(11):1837-1848

The excitatory amino acid, (S)-glutamic acid, plays an important role in controlling many neuronal processes. Its action is mediated by two main groups of receptors: the ionotropic receptors (which include NMDA, AMPA and kainic acid subtypes) and the metabotropic receptors (mGluR(1-8)) mediating G-protein coupled responses. This review focuses on the strychnine insensitive glycine binding site located on the NMDA receptor channel, and on the possible use of selective antagonists for the treatment of stroke. Stroke is a devastating disease caused by a sudden vascular accident. Neurochemically, a massive release of glutamate occurs in neuronal tissue; this overactivates the NMDA receptor, leading to increased intracellular calcium influx, which causes neuronal cell death through necrosis. NMDA receptor activation strongly depends upon the presence of glycine as a co-agonist. Therefore, the administration of a glycine antagonist can block overactivation of NMDA receptors, thus preserving neurones from damage. The glycine antagonists currently identified can be divided into five main categories depending on their chemical structure: indoles, tetrahydroquinolines, benzoazepines, quinoxalinediones and pyrida-zinoquinolines.

Monopoli A, Lozza G, Forlani A, Mattavelli A, Ongini E. Blockade of adenosine A2A receptors by SCH 58261 results in neuroprotective effects in cerebral ischaemia in rats. Neuroreport Dec. 1, 1998;9(17):3955-9

Blockade of adenosine receptors can reduce cerebral infarct size in the model of global ischaemia. Using the potent and selective A2A adenosine receptor antagonist, SCH 58261, we assessed whether A2A receptors are involved in the neuronal damage following focal cerebral ischaemia as induced by occluding the left middle cerebral artery. SCH 58261 (0.01 mg/kg either i.p. or i.v.) administered to normotensive rats 10 min after ischaemia markedly reduced cortical infarct volume as measured 24 h later (30% vs controls, p<0.05). Similar effects were observed when SCH 58261 (0.01 mg/kg, i.p.) was administered to hypertensive rats (28% infarct volume reduction vs controls, p<0.05). Neuroprotective properties of SCH 58261 administered after ischaemia indicate that blockade of A2A adenosine receptors is a potentially useful biological target for the reduction of brain injury.

Panel 1.3D Summary: Ag2973 Expression of the NOV20 gene is low/undetectable in all samples on this panel (CTs>35). (Data not shown.)

Panel 3D Summary: Ag2973 Expression of the NOV20 gene is low/undetectable in all samples on this panel (CTs>35). (Data not shown.)

Panel 4D Summary: Ag2973 Significant expression of the NOV20 gene is detected in a liver cirrhosis sample (CT=32.7). Furthermore, expression of this gene is not detected in normal liver in Panel 1.3D, suggesting that its expression is unique to liver cirrhosis. This gene encodes a putative GPCR; therefore, antibodies or small molecule therapeutics could reduce or inhibit fibrosis that occurs in liver cirrhosis. In addition, antibodies to this putative GPCR could also be used for the diagnosis of liver cirrhosis.

Panel CNS_{—}1 Summary: Ag2973 Expression of the NOV20 gene is low/undetectable in all samples on this panel (CTs>35). (Data not shown.)

NOV16b

Expression of gene NOV16b was assessed using the primer-probe sets Ag2875 and Ag3010, described in Tables QA and QB. Results of the RTQ-PCR runs are shown in Table QC.

TABLE QA
Probe Name Ag2875

| | | | Start | |
|---|---|---|---|---|
| Primers | Sequence | Length | Position | SEQ ID NO: |
| Forward | 5′-gaacatcatctcctaccctgaa-3′ | 22 | 267 | 1051 |
| Probe | TET-5′-tgcatgactcagctctacttcttcctcg-3′-TAMRA | 28 | 289 | 1052 |
| Reverse | 5′-atgtgacactctgcaatagcaa-3′ | 20 | 320 | 1053 |

TABLE QB
Probe Name Ag3010

| | | | Start | |
|---|---|---|---|---|
| Primers | Sequence | Length | Position | SEQ ID NO: |
| Forward | 5′-ggggaaagtatcctctgtgttt-3′ | 22 | 810 | 1054 |
| Probe | TET-5′-ttattgtgcccatgttgaaccctctg-3′-TAMRA | 26 | 839 | 1055 |
| Reverse | 5′-cagggaaacatggacatcttta-3′ | 22 | 882 | 1056 |

TABLE QC
Panel 4D

| | Rel. Exp. (%) | | Rel. Exp. (%) |
|---|---|---|---|
| | Ag2875, Run | | Ag2875, Run |
| Tissue Name | 164311029 | Tissue Name | 164311029 |
| Secondary Th1 act | 0.0 | HUVEC IL-1beta | 0.0 |
| Secondary Th2 act | 0.0 | HUVEC IFN gamma | 3.0 |
| Secondary Tr1 act | 0.0 | HUVEC TNF alpha + IFN | 0.0 |
| | | gamma | |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC | 0.0 |
| | | none | |
| Primary Th1 act | 0.0 | Lung Microvascular EC | 0.0 |
| | | TNF alpha + IL-1beta | |
| Primary Th2 act | 0.0 | Microvascular Dermal EC | 0.0 |
| | | none | |
| Primary Tr1 act | 0.0 | Microsvasular Dermal EC | 0.0 |
| | | TNF alpha + IL-1beta | |
| Primary Th1 rest | 0.0 | Bronchial epithelium | 0.0 |
| | | TNF alpha + IL1beta | |
| Primary Th2 rest | 0.0 | Small airway epithelium | 0.0 |
| | | none | |
| Primary Tr1 rest | 0.0 | Small airway epithelium | 0.0 |
| | | TNF alpha + IL-1beta | |
| CD45RA CD4 | 0.0 | Coronery artery SMC rest | 0.0 |
| lymphocyte act | | | |
| CD45RO CD4 | 0.0 | Coronery artery SMC | 0.0 |
| lymphocyte act | | TNF alpha + IL-1beta | |
| CD8 lymphocyte act | 0.0 | Astrocytes rest | 0.0 |
| Secondary CD8 | 0.0 | Astrocytes TNF alpha + | 0.0 |
| lymphocyte rest | | IL-1beta | |
| Secondary CD8 | 0.0 | KU-812 (Basophil) rest | 0.0 |
| lymphocyte act | | | |
| CD4 lymphocyte none | 0.0 | KU-812 (Basophil) | 24.0 |
| | | PMA/ionomycin | |
| 2ry Th1/Th2/Tr1_anti- | 5.1 | CCD1106 (Keratinocytes) | 0.0 |
| CD95 CH11 | | none | |
| LAK cells rest | 0.0 | CCD1106 (Keratinocytes) | 0.0 |
| | | TNF alpha + IL-1beta | |
| LAK cells IL-2 | 0.0 | Liver cirrhosis | 72.2 |
| LAK cells IL-2 + IL-12 | 0.0 | Lupus kidney | 0.0 |
| LAK cells IL-2 + IFN | 0.0 | NCI-H292 none | 20.7 |
| gamma | | | |
| LAK cells IL-2 + IL-18 | 0.0 | NCI-H292 IL-4 | 0.0 |
| LAK cells | 0.0 | NCI-H292 IL-9 | 0.0 |
| PMA/ionomycin | | | |
| NK Cells IL-2 rest | 0.0 | NCI-H292 IL-13 | 0.0 |
| Two Way MLR 3 day | 0.0 | NCI-H292 IFN gamma | 0.0 |
| Two Way MLR 5 day | 0.0 | HPAEC none | 0.0 |
| Two Way MLR 7 day | 0.0 | HPAEC TNF alpha + IL- | 1.4 |
| | | 1beta | |
| PBMC rest | 0.0 | Lung fibroblast none | 0.0 |
| PBMC PWM | 0.0 | Lung fibroblast TNF alpha + | 0.0 |
| | | IL-1beta | |
| PBMC PHA-L | 0.0 | Lung fibroblast IL-4 | 0.0 |
| Ramos (B cell) none | 0.0 | Lung fibroblast IL-9 | 0.0 |
| Ramos (B cell) | 0.0 | Lung fibroblast IL-13 | 0.0 |
| ionomycin | | | |
| B lymphocytes PWM | 0.0 | Lung fibroblast IFN | 3.2 |
| | | gamma | |
| B lymphocytes CD40L | 0.0 | Dermal fibroblast | 0.0 |
| and IL-4 | | CCD1070 rest | |
| EOL-1 dbcAMP | 3.1 | Dermal fibroblast | 0.0 |
| | | CCD1070 TNF alpha | |
| EOL-1 dbcAMP | 0.0 | Dermal fibroblast | 0.0 |
| PMA/ionomycin | | CCD1070 IL-1beta | |
| Dendritic cells none | 33.2 | Dermal fibroblast IFN | 0.0 |
| | | gamma | |
| Dendritic cells LPS | 2.4 | Dermal fibroblast IL-4 | 1.9 |
| Dendritic cells anti- | 100.0 | IBD Colitis 2 | 16.3 |
| CD40 | | | |
| Monocytes rest | 0.0 | IBD Crohn's | 6.7 |
| Monocytes LPS | 0.0 | Colon | 0.0 |
| Macrophages rest | 44.1 | Lung | 21.8 |
| Macrophages LPS | 0.0 | Thymus | 0.0 |
| HUVEC none | 3.1 | Kidney | 0.0 |
| HUVEC starved | 0.0 | | |

Panel 1.3D Summary: Ag2875/Ag3010 Results from two experiments with the NOV16b gene are not included. The amp plots indicate that there were experimental difficulties with these runs.

Panel 2.2 Summary: Ag2875 Expression of the NOV16b gene is low/undetectable in all samples on this panel (CTs>35). (Data not shown.)

Panel 4D Summary: Ag2875 Highest expression of the NOV15a is in anti-CD40 treated dendritic cells (CT=33.2), with much lower expression in untreated dendritic cells. Thus, this gene product may be important in dendritic cell activation. Significant expression of this gene is also seen in liver cirrhosis. This gene encodes a putative GPCR; therefore, antibodies or small molecule therapeutics could reduce or inhibit fibrosis that occurs in liver cirrhosis. In addition, antibodies to this putative GPCR could also be used for the diagnosis of liver cirrhosis. In addition, significant expression of this gene is seen in resting macrophages. The putative GPCR encoded for by this transcript may therefore be important in macrophage detection of chemokine gradients and trafficking into specific sites within a tissue and in activation. Antibody or protein therapeutics designed against the protein encoded for by this transcript could reduce or inhibit inflammation in asthma, emphysema, allergy, psoriasis, arthritis, or any other condition in which macrophage localization/activation is important. A second experiment with the probe/primer set Ag3010 shows low/undetectable expression in all samples on this panel (CTs>35). (Data not shown.)

NOV21a

Expression of gene NOV21a was assessed using the primer-probe sets Ag2963 and Ag1292, described in Tables RA and RB. Results of the RTQ-PCR runs are shown in Tables RC, RD and RE.

TABLE RA
Probe Name Ag2963

| | | | Start | |
|---|---|---|---|---|
| Primers | Sequence | Length | Position | SEQ ID NO: |
| Forward | 5′-ggtaccaccctttgttccaat-3′ | 21 | 903 | 1057 |
| Probe | TET-5′-aaacacagcccctccaccctagct-3′-TAMRA | 24 | 924 | 1058 |
| Reverse | 5′-gtcctcgctgtgacactga-3′ | 19 | 963 | 1059 |

TABLE RB
Probe Name Ag1292

| | | | Start | |
|---|---|---|---|---|
| Primers | Sequence | Length | Position | SEQ ID NO: |
| Forward | 5′-cagctgtgagaggctggata-3′ | 20 | 731 | 1060 |
| Probe | TET-5′-atccaggagtgaccaccacgtgact-3′-TAMRA | 25 | 760 | 1061 |
| Reverse | 5′-tcctgttgctttcacgtagagt-3′ | 22 | 797 | 1062 |

TABLE RC
CNS_neurodegeneration_v1.0

| | Rel. Exp. (%) | Rel. Exp. (%) | | Rel. Exp. (%) | Rel. Exp. (%) |
|---|---|---|---|---|---|
| | Ag2963, Run | Ag2963, Run | Tissue | Ag2963, Run | Ag2963, Run |
| Tissue Name | 209778824 | 230512509 | Name | 209778824 | 230512509 |
| AD 1 Hippo | 6.5 | 6.8 | Control | 6.1 | 3.6 |
| | | | (Path) 3 | | |
| | | | Temporal | | |
| | | | Ctx | | |
| AD 2 Hippo | 21.2 | 16.8 | Control | 32.5 | 26.2 |
| | | | (Path) 4 | | |
| | | | Temporal | | |
| | | | Ctx | | |
| AD 3 Hippo | 5.5 | 6.3 | AD 1 | 5.3 | 4.9 |
| | | | Occipital | | |
| | | | Ctx | | |
| AD 4 Hippo | 7.4 | 7.9 | AD 2 | 0.0 | 0.0 |
| | | | Occipital | | |
| | | | Ctx | | |
| | | | (Missing) | | |
| AD 5 hippo | 21.0 | 22.2 | AD 3 | 7.9 | 6.2 |
| | | | Occipital | | |
| | | | Ctx | | |
| AD 6 Hippo | 12.2 | 19.6 | AD 4 | 12.1 | 13.8 |
| | | | Occipital | | |
| | | | Ctx | | |
| Control 2 | 58.6 | 44.8 | AD 5 | 6.7 | 3.3 |
| Hippo | | | Occipital | | |
| | | | Ctx | | |
| Control 4 | 12.9 | 12.9 | AD 6 | 73.7 | 81.8 |
| Hippo | | | Occipital | | |
| | | | Ctx | | |
| Control (Path) | 2.0 | 4.5 | Control 1 | 5.9 | 2.5 |
| 3 Hippo | | | Occipital | | |
| | | | Ctx | | |
| AD 1 Temporal | 2.3 | 6.3 | Control 2 | 89.5 | 100.0 |
| Ctx | | | Occipital | | |
| | | | Ctx | | |
| AD 2 Temporal | 13.2 | 19.5 | Control 3 | 27.4 | 25.7 |
| Ctx | | | Occipital | | |
| | | | Ctx | | |
| AD 3 Temporal | 3.3 | 7.1 | Control 4 | 11.3 | 12.4 |
| Ctx | | | Occipital | | |
| | | | Ctx | | |
| AD 4 Temporal | 15.8 | 20.7 | Control | 100.0 | 93.3 |
| Ctx | | | (Path) 1 | | |
| | | | Occipital | | |
| | | | Ctx | | |
| AD 5 Inf | 16.7 | 21.5 | Control | 8.4 | 11.0 |
| Temporal Ctx | | | (Path) 2 | | |
| | | | Occipital | | |
| | | | Ctx | | |
| AD 5 | 12.5 | 11.8 | Control | 4.4 | 2.2 |
| SupTemporal | | | (Path) 3 | | |
| Ctx | | | Occipital | | |
| | | | Ctx | | |
| AD 6 Inf | 25.0 | 25.2 | Control | 16.7 | 20.6 |
| Temporal Ctx | | | (Path) 4 | | |
| | | | Occipital | | |
| | | | Ctx | | |
| AD 6 Sup | 22.7 | 17.1 | Control 1 | 9.8 | 4.6 |
| Temporal Ctx | | | Parietal Ctx | | |
| Control 1 | 2.1 | 6.1 | Control 2 | 8.2 | 8.4 |
| Temporal Ctx | | | Parietal Ctx | | |
| Control 2 | 82.4 | 51.8 | Control 3 | 26.6 | 24.7 |
| Temporal Ctx | | | Parietal Ctx | | |
| Control 3 | 31.2 | 20.2 | Control | 94.6 | 66.9 |
| Temporal Ctx | | | (Path) 1 | | |
| | | | Parietal Ctx | | |
| Control 4 | 11.6 | 12.9 | Control | 19.3 | 24.8 |
| Temporal Ctx | | | (Path) 2 | | |
| | | | Parietal Ctx | | |
| Control (Path) | 77.9 | 56.3 | Control | 2.6 | 3.8 |
| 1 Temporal Ctx | | | (Path) 3 | | |
| | | | Parietal Ctx | | |
| Control (Path) | 17.0 | 12.3 | Control | 35.8 | 23.0 |
| 2 Temporal Ctx | | | (Path) 4 | | |
| | | | Parietal Ctx | | |

TABLE RD
Panel 1.3D

| | Rel. Exp. (%) Ag2963, | | Rel. Exp. (%) Ag2963, |
|---|---|---|---|
| Tissue Name | Run 167809191 | Tissue Name | Run 167809191 |
| Liver adenocarcinoma | 82.4 | Kidney (fetal) | 45.4 |
| Pancreas | 50.0 | Renal ca. 786-0 | 53.6 |
| Pancreatic ca. CAPAN 2 | 10.7 | Renal ca. A498 | 19.6 |
| Adrenal gland | 14.2 | Renal ca. RXF 393 | 21.2 |
| Thyroid | 26.2 | Renal ca. ACHN | 12.5 |
| Salivary gland | 21.2 | Renal ca. UO-31 | 13.7 |
| Pituitary gland | 31.6 | Renal ca. TK-10 | 27.2 |
| Brain (fetal) | 40.9 | Liver | 49.7 |
| Brain (whole) | 71.7 | Liver (fetal) | 45.1 |
| Brain (amygdala) | 11.6 | Liver ca. | 16.3 |
| | | (hepatoblast) HepG2 | |
| Brain (cerebellum) | 62.4 | Lung | 26.4 |
| Brain (hippocampus) | 47.0 | Lung (fetal) | 66.0 |
| Brain (substantia nigra) | 89.5 | Lung ca. (small cell) | 42.0 |
| | | LX-1 | |
| Brain (thalamus) | 53.2 | Lung ca. (small cell) | 21.6 |
| | | NCI-H69 | |
| Cerebral Cortex | 92.7 | Lung ca. (s.cell var.) | 59.9 |
| | | SHP-77 | |
| Spinal cord | 30.6 | Lung ca. (large | 12.9 |
| | | cell)NCI-H460 | |
| glio/astro U87-MG | 28.5 | Lung ca. (non-sm. | 37.1 |
| | | cell) A549 | |
| glio/astro U-118-MG | 44.4 | Lung ca. (non-s.cell) | 47.3 |
| | | NCI-H23 | |
| astrocytoma SW1783 | 50.3 | Lung ca. (non-s.cell) | 11.8 |
| | | HOP-62 | |
| neuro*; met SK-N-AS | 40.6 | Lung ca. (non-s.cl) | 16.8 |
| | | NCI-H522 | |
| astrocytoma SF-539 | 37.1 | Lung ca. (squam.) | 29.3 |
| | | SW 900 | |
| astrocytoma SNB-75 | 49.3 | Lung ca. (squam.) | 51.1 |
| | | NCI-H596 | |
| glioma SNB-19 | 31.0 | Mammary gland | 70.2 |
| glioma U251 | 52.9 | Breast ca.* (pl.ef) | 15.2 |
| | | MCF-7 | |
| glioma SF-295 | 27.0 | Breast ca.* (pl.ef) | 29.7 |
| | | MDA-MB-231 | |
| Heart (fetal) | 90.8 | Breast ca.* (pl.ef) | 41.2 |
| | | T47D | |
| Heart | 22.7 | Breast ca. BT-549 | 28.3 |
| Skeletal muscle (fetal) | 83.5 | Breast ca. MDA-N | 54.3 |
| Skeletal muscle | 57.0 | Ovary | 47.0 |
| Bone marrow | 25.2 | Ovarian ca. OVCAR-3 | 26.1 |
| Thymus | 74.7 | Ovarian ca. OVCAR-4 | 18.7 |
| Spleen | 28.1 | Ovarian ca. OVCAR-5 | 82.9 |
| Lymph node | 54.7 | Ovarian ca. OVCAR-8 | 3.4 |
| Colorectal | 7.9 | Ovarian ca. IGROV-1 | 22.2 |
| Stomach | 7.0 | Ovarian ca.* (ascites) | 100.0 |
| | | SK-OV-3 | |
| Small intestine | 25.3 | Uterus | 28.3 |
| Colon ca. SW480 | 25.9 | Placenta | 3.2 |
| Colon ca.* | 40.1 | Prostate | 22.1 |
| SW620(SW480 met) | | | |
| Colon ca. HT29 | 24.1 | Prostate ca.* (bone | 29.3 |
| | | met)PC-3 | |
| Colon ca. HCT-116 | 35.8 | Testis | 19.3 |
| Colon ca. CaCo-2 | 52.9 | Melanoma | 19.5 |
| | | Hs688(A).T | |
| Colon ca. | 4.4 | Melanoma* (met) | 22.4 |
| tissue(ODO3866) | | Hs688(B).T | |
| Colon ca. HCC-2998 | 31.9 | Melanoma UACC-62 | 42.9 |
| Gastric ca.* (liver met) | 27.2 | Melanoma M14 | 8.3 |
| NCI-N87 | | | |
| Bladder | 20.6 | Melanoma LOX | 25.0 |
| | | IMVI | |
| Trachea | 11.7 | Melanoma* (met) | 23.3 |
| | | SK-MEL-5 | |
| Kidney | 56.6 | Adipose | 21.0 |

TABLE RE
Panel 4D

| | Rel. | Rel. | | Rel. | Rel. |
|---|---|---|---|---|---|
| | Exp. (%) | Exp. (%) | | Exp. (%) | Exp. (%) |
| | Ag1292, | Ag2963, | | Ag1292, | Ag2963, |
| | Run | Run | | Run | Run |
| Tissue Name | 138719232 | 164333805 | Tissue Name | 138719232 | 164333805 |
| Secondary Th1 act | 14.5 | 18.6 | HUVEC IL-1beta | 7.6 | 3.0 |
| Secondary Th2 act | 11.3 | 47.0 | HUVEC IFN | 13.5 | 31.6 |
| | | | gamma | | |
| Secondary Tr1 act | 23.2 | 29.1 | HUVEC TNF | 2.2 | 26.1 |
| | | | alpha + IFN | | |
| | | | gamma | | |
| Secondary Th1 rest | 4.0 | 8.8 | HUVEC TNF | 8.3 | 33.2 |
| | | | alpha + IL4 | | |
| Secondary Th2 rest | 5.0 | 18.2 | HUVEC IL-11 | 2.8 | 26.6 |
| Secondary Tr1 rest | 5.8 | 2.2 | Lung | 7.1 | 62.0 |
| | | | Microvascular EC | | |
| | | | none | | |
| Primary Th1 act | 19.1 | 55.9 | Lung | 2.7 | 40.9 |
| | | | Microvascular EC | | |
| | | | TNF alpha + IL- | | |
| | | | 1beta | | |
| Primary Th2 act | 16.4 | 33.4 | Microvascular | 8.8 | 41.2 |
| | | | Dermal EC none | | |
| Primary Tr1 act | 25.3 | 66.9 | Microsvasular | 7.5 | 31.6 |
| | | | Dermal EC | | |
| | | | TNF alpha + IL- | | |
| | | | 1beta | | |
| Primary Th1 rest | 13.6 | 33.0 | Bronchial | 8.7 | 37.1 |
| | | | epithelium | | |
| | | | TNF alpha + | | |
| | | | IL1beta | | |
| Primary Th2 rest | 9.3 | 16.7 | Small airway | 1.7 | 8.4 |
| | | | epithelium none | | |
| Primary Tr1 rest | 18.8 | 23.0 | Small airway | 6.6 | 14.2 |
| | | | epithelium | | |
| | | | TNF alpha + IL- | | |
| | | | 1beta | | |
| CD45RA CD4 | 7.9 | 39.0 | Coronery artery | 55.9 | 21.6 |
| lymphocyte act | | | SMC rest | | |
| CD45RO CD4 | 10.3 | 27.2 | Coronery artery | 5.3 | 14.6 |
| lymphocyte act | | | SMC TNF alpha + | | |
| | | | IL-1beta | | |
| CD8 lymphocyte | 9.9 | 29.7 | Astrocytes rest | 5.8 | 24.1 |
| act | | | | | |
| Secondary CD8 | 11.9 | 44.4 | Astrocytes | 4.4 | 19.5 |
| lymphocyte rest | | | TNF alpha + IL- | | |
| | | | 1beta | | |
| Secondary CD8 | 13.0 | 22.8 | KU-812 | 16.0 | 66.9 |
| lymphocyte act | | | (Basophil) rest | | |
| CD4 lymphocyte | 6.3 | 21.3 | KU-812 | 8.1 | 87.7 |
| none | | | (Basophil) | | |
| | | | PMA/ionomycin | | |
| 2ry | 7.9 | 20.3 | CCD1106 | 5.8 | 41.2 |
| Th1/Th2/Tr1_anti- | | | (Keratinocytes) | | |
| CD95 CH11 | | | none | | |
| LAK cells rest | 10.1 | 39.5 | CCD1106 | 6.5 | 11.5 |
| | | | (Keratinocytes) | | |
| | | | TNF alpha + IL- | | |
| | | | 1beta | | |
| LAK cells IL-2 | 5.7 | 33.2 | Liver cirrhosis | 4.9 | 7.9 |
| LAK cells IL-2 + IL- | 9.0 | 34.2 | Lupus kidney | 4.5 | 9.2 |
| 12 | | | | | |
| LAK cells IL- | 12.4 | 31.9 | NCI-H292 none | 11.5 | 63.7 |
| 2 + IFN gamma | | | | | |
| LAK cells IL-2 + | 16.8 | 16.2 | NCI-H292 IL-4 | 17.7 | 77.9 |
| IL-18 | | | | | |
| LAK cells | 15.4 | 50.7 | NCI-H292 IL-9 | 59.5 | 54.3 |
| PMA/ionomycin | | | | | |
| NK Cells IL-2 rest | 7.6 | 22.8 | NCI-H292 IL-13 | 44.1 | 63.7 |
| Two Way MLR 3 | 7.7 | 49.0 | NCI-H292 IFN | 10.4 | 89.5 |
| day | | | gamma | | |
| Two Way MLR 5 | 5.4 | 29.9 | HPAEC none | 8.2 | 28.1 |
| day | | | | | |
| Two Way MLR 7 | 5.3 | 17.0 | HPAEC TNF | 9.6 | 30.1 |
| day | | | alpha + IL-1beta | | |
| PBMC rest | 5.2 | 11.2 | Lung fibroblast | 8.7 | 28.7 |
| | | | none | | |
| PBMC PWM | 17.1 | 100.0 | Lung fibroblast | 4.5 | 6.9 |
| | | | TNF alpha + IL-1 | | |
| | | | beta | | |
| PBMC PHA-L | 10.9 | 51.4 | Lung fibroblast | 10.2 | 53.6 |
| | | | IL-4 | | |
| Ramos (B cell) | 24.1 | 88.3 | Lung fibroblast | 9.7 | 49.7 |
| none | | | IL-9 | | |
| Ramos (B cell) | 14.6 | 92.7 | Lung fibroblast | 22.1 | 44.1 |
| ionomycin | | | IL-13 | | |
| B lymphocytes | 11.8 | 59.0 | Lung fibroblast | 9.5 | 29.5 |
| PWM | | | IFN gamma | | |
| B lymphocytes | 20.7 | 21.8 | Dermal fibroblast | 32.3 | 37.6 |
| CD40L and IL-4 | | | CCD1070 rest | | |
| EOL-1 dbcAMP | 23.3 | 32.8 | Dermal fibroblast | 100.0 | 39.0 |
| | | | CCD1070 TNF | | |
| | | | alpha | | |
| EOL-1 dbcAMP | 4.2 | 25.5 | Dermal fibroblast | 14.3 | 28.7 |
| PMA/ionomycin | | | CCD1070 IL-1 | | |
| | | | beta | | |
| Dendritic cells | 4.3 | 22.8 | Dermal fibroblast | 7.5 | 17.8 |
| none | | | IFN gamma | | |
| Dendritic cells LPS | 4.4 | 10.5 | Dermal fibroblast | 30.4 | 48.6 |
| | | | IL-4 | | |
| Dendritic cells anti- | 6.4 | 24.3 | IBD Colitis 2 | 1.3 | 1.2 |
| CD40 | | | | | |
| Monocytes rest | 7.6 | 38.4 | IBD Crohn's | 2.6 | 2.2 |
| Monocytes LPS | 4.5 | 8.0 | Colon | 7.3 | 37.9 |
| Macrophages rest | 6.3 | 21.5 | Lung | 5.8 | 22.8 |
| Macrophages LPS | 5.5 | 1.9 | Thymus | 12.7 | 39.2 |
| HUVEC none | 4.8 | 30.8 | Kidney | 19.1 | 98.6 |
| HUVEC starved | 7.8 | 27.7 | | | |

CNS_neurodegeneration_v1.0 Summary: Ag2963 The NOV21a gene, a secretory protease homolog, appears to be downregulated in the temporal cortex of Alzheimer's disease patients when compared to non-demented controls. Up regulation of this protease may therefore be of use in the treatment of Alzheimer's, particularly because Alzheimer's disease is believed to result at least in part from the improper processing of proteins (APP, Tau). This protease may serve to lower the levels of these disease proteins and ameliorate the dementia/pathology associated with Alzheimer's.

Panel 1.3D Summary: Ag2963 The NOV21a gene, a putative secretory serine-protease, is widely expressed in this panel. Highest expression is in an ovarian cancer cell line (CT=32), with expression detected in all cancer cell lines in this panel. Thus, inhibition of the protease domain might lead to a decrease in cell survival and proliferation and serve as a small molecule target in cancer.

This gene product also has low levels of expression in pancreas, thyroid, pituitary, adipose, and adult and fetal types of heart, skeletal muscle and liver. Therefore, this serine protease-like gene product may be a small molecule target for the treatment of endocrine and metabolic diseases, including obesity and Types 1 and 2 diabetes.

The expression in this panel further confirms expression of this gene in the CNS. Please see CNS_neurodegeneration for discussion of utility of this gene in the CNS.

Panel 4D Summary: Ag1292/Ag2693 The NOV21a transcript is expressed on most tissues in panel 4D. This widespread expression is consistent with the results in Panel 1.3D. This transcript encodes a serine protease like protein with potential enzymatic activity and may important in maintaining normal cellular functions in a number of tissues. Therefore, therapies designed with the protein encoded by this transcript could be important in regulating cellular viability or function.

NOV22a

Expression of gene NOV22a was assessed using the primer-probe set Ag2964, described in Table SA.

TABLE SA
Probe Name Ag2964

| | | | Start | |
|---|---|---|---|---|
| Primers | Sequence | Length | Position | SEQ ID NO: |
| Forward | 5′-gaaatacgcaggaaggaaatct-3′ | 22 | 473 | 1063 |
| Probe | TET-5′-cgtgggcatcatagaccagaaaacct-3′-TAMRA | 26 | 507 | 1064 |
| Reverse | 5′-ggtctgtgagggagaagttgta-3′ | 22 | 544 | 1065 |

CNS_neurodegeneration_v1.0 Summary: Ag2964 Expression of the NOV22a gene is low/undetectable in all samples on this panel (CTs>35). (Data not shown.) The amp plot indicates that there is a high probability of a probe failure.

Panel 1.3D Summary: Ag2964 Expression of the NOV22a gene is low/undetectable in all samples on this panel (CTs>35). (Data not shown.) The amp plot indicates that there is a high probability of a probe failure.

Panel 4D Summary: Ag2964 Expression of the NOV22a gene is low/undetectable in all samples on this panel (CTs>35). (Data not shown.) The amp plot indicates that there is a high probability of a probe failure.

NOV23a

Expression of gene NOV23a was assessed using the primer-probe set Ag2967, described in Table TA.

TABLE SA
Probe Name Ag2964

| | | | Start | |
|---|---|---|---|---|
| Primers | Sequence | Length | Position | SEQ ID NO: |
| Forward | 5′-ctcaaggaggacttccgtaag-3′ | 21 | 478 | 1066 |
| Probe | TET-5′-atcttcctctgcagaaagccactgtg-3′-TAMRA | 26 | 500 | 1067 |
| Reverse | 5′-agtgagtgaatggccgtaca-3′ | 20 | 557 | 1068 |

CNS_neurodegeneration_v1.0 Summary: Ag2967 Expression of the NOV23a gene is low/undetectable in all samples on this panel (CTs>35). (Data not shown.) The amp plot indicates that there is a high probability of a probe failure.

Panel 1.3D Summary: Ag2967 Expression of the NOV23a gene is low/undetectable in all samples on this panel (CTs>35). (Data not shown.) The amp plot indicates that there is a high probability of a probe failure.

Panel 4D Summary: Ag2967 Expression of the NOV23a gene is low/undetectable in all samples on this panel (CTs>35). (Data not shown.) The amp plot indicates that there is a high probability of a probe failure.

NOV23a and NOV23b

Expression of gene NOV23a and variant NOV23b was assessed using the primer-probe set Ag2996, described in Table UA. Results of the RTQ-PCR runs are shown in Table UB.

TABLE UA
Probe Name Ag2996

| | | | Start | |
|---|---|---|---|---|
| Primers | Sequence | Length | Position | SEQ ID NO: |
| Forward | 5′-cagaggttacgccctgaagt-3′ | 20 | 54 | 1069 |
| Probe | TET-5′-ctcagatcctgggccaggcactg-3′-TAMRA | 23 | 77 | 1070 |
| Reverse | 5′-cagaaagaggtcctgctcatg-3′ | 21 | 113 | 1071 |

TABLE UB
Panel 4D

| | Rel. Exp. (%) | | Rel. Exp. (%) |
|---|---|---|---|
| | Ag2996, Run | | Ag2996, Run |
| Tissue Name | 165296353 | Tissue Name | 165296353 |
| Secondary Th1 act | 0.0 | HUVEC IL-1beta | 0.0 |
| Secondary Th2 act | 0.0 | HUVEC IFN gamma | 0.0 |
| Secondary Tr1 act | 0.0 | HUVEC TNF alpha + IFN | 0.0 |
| | | gamma | |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC | 23.0 |
| | | none | |
| Primary Th1 act | 0.0 | Lung Microvascular EC | 0.0 |
| | | TNF alpha + IL-1beta | |
| Primary Th2 act | 0.0 | Microvascular Dermal EC | 0.0 |
| | | none | |
| Primary Tr1 act | 0.0 | Microsvasular Dermal EC | 0.0 |
| | | TNF alpha + IL-1beta | |
| Primary Th1 rest | 0.0 | Bronchial epithelium | 0.0 |
| | | TNF alpha + IL1beta | |
| Primary Th2 rest | 0.0 | Small airway epithelium | 0.0 |
| | | none | |
| Primary Tr1 rest | 0.0 | Small airway epithelium | 0.0 |
| | | TNF alpha + IL-1beta | |
| CD45RA CD4 | 0.0 | Coronery artery SMC rest | 0.0 |
| lymphocyte act | | | |
| CD45RO CD4 | 0.0 | Coronery artery SMC | 0.0 |
| lymphocyte act | | TNF alpha + IL-1beta | |
| CD8 lymphocyte act | 0.0 | Astrocytes rest | 0.0 |
| Secondary CD8 | 0.0 | Astrocytes TNF alpha + | 0.0 |
| lymphocyte rest | | IL-1beta | |
| Secondary CD8 | 0.0 | KU-812 (Basophil) rest | 0.0 |
| lymphocyte act | | | |
| CD4 lymphocyte none | 0.0 | KU-812 (Basophil) | 0.0 |
| | | PMA/ionomycin | |
| 2ry Th1/Th2/Tr1_anti- | 0.0 | CCD1106 (Keratinocytes) | 0.0 |
| CD95 CH11 | | none | |
| LAK cells rest | 0.0 | CCD1106 (Keratinocytes) | 0.0 |
| | | TNF alpha + IL-1beta | |
| LAK cells IL-2 | 0.0 | Liver cirrhosis | 100.0 |
| LAK cells IL-2 + IL-12 | 0.0 | Lupus kidney | 0.0 |
| LAK cells IL-2 + IFN | 0.0 | NCI-H292 none | 0.0 |
| gamma | | | |
| LAK cells IL-2 + IL-18 | 0.0 | NCI-H292 IL-4 | 0.0 |
| LAK cells | 0.0 | NCI-H292 IL-9 | 0.0 |
| PMA/ionomycin | | | |
| NK Cells IL-2 rest | 0.0 | NCI-H292 IL-13 | 0.0 |
| Two Way MLR 3 day | 0.0 | NCI-H292 IFN gamma | 0.0 |
| Two Way MLR 5 day | 0.0 | HPAEC none | 0.0 |
| Two Way MLR 7 day | 0.0 | HPAEC TNF alpha + IL- | 0.0 |
| | | 1beta | |
| PBMC rest | 0.0 | Lung fibroblast none | 0.0 |
| PBMC PWM | 11.3 | Lung fibroblast TNF alpha + | 0.0 |
| | | IL-1beta | |
| PBMC PHA-L | 0.0 | Lung fibroblast IL-4 | 0.0 |
| Ramos (B cell) none | 14.6 | Lung fibroblast IL-9 | 0.0 |
| Ramos (B cell) | 0.0 | Lung fibroblast IL-13 | 0.0 |
| ionomycin | | | |
| B lymphocytes PWM | 0.0 | Lung fibroblast IFN | 0.0 |
| | | gamma | |
| B lymphocytes CD40L | 15.3 | Dermal fibroblast | 0.0 |
| and IL-4 | | CCD1070 rest | |
| EOL-1 dbcAMP | 0.0 | Dermal fibroblast | 0.0 |
| | | CCD1070 TNF alpha | |
| EOL-1 dbcAMP | 0.0 | Dermal fibroblast | 0.0 |
| PMA/ionomycin | | CCD1070 IL-1beta | |
| Dendritic cells none | 0.0 | Dermal fibroblast IFN | 0.0 |
| | | gamma | |
| Dendritic cells LPS | 0.0 | Dermal fibroblast IL-4 | 0.0 |
| Dendritic cells anti- | 0.0 | IBD Colitis 2 | 0.0 |
| CD40 | | | |
| Monocytes rest | 0.0 | IBD Crohn's | 0.0 |
| Monocytes LPS | 0.0 | Colon | 0.0 |
| Macrophages rest | 0.0 | Lung | 49.0 |
| Macrophages LPS | 0.0 | Thymus | 0.0 |
| HUVEC none | 0.0 | Kidney | 0.0 |
| HUVEC starved | 0.0 | | |

Panel 1.3D Summary: Ag2996 Results from one experiment with the NOV23a gene are not included. The amp plot indicates that there were experimental difficulties with this run.

Panel 4D Summary: Ag2996 Significant expression of the NOV23a gene is detected in a liver cirrhosis sample and normal lung tissue(CTs=33-35). Thus, antibodies to this protein product could potentially be used for the diagnosis of liver cirrhosis or as a marker of normal lung tissue. Furthermore, therapeutic modulation of the expression or function of this gene may reduce or inhibit fibrosis that occurs in liver cirrhosis.

NOV24a

Expression of gene NOV24a was assessed using the primer-probe set Ag2934, described in Table VA.

TABLE VA
Probe Name Ag2934

| | | | Start | |
|---|---|---|---|---|
| Primers | Sequence | Length | Position | SEQ ID NO: |
| Forward | 5′-gaatctaattccgcaggaaatg-3′ | 22 | 1645 | 1072 |
| Probe | TET-5′-cgtgtttgccaaattctcagcgttta-3′-TAMRA | 26 | 1668 | 1073 |
| Reverse | 5′-tcttttcatgaacctcatttgc-3′ | 22 | 1714 | 1074 |

CNS_neurodegeneration_v1.0 Summary: Ag2934 Expression of the NOV24a gene is low/undetectable in all samples on this panel (CTs>35). (Data not shown.)

Panel 1.3D Summary: Ag2934 Expression of the NOV24a gene is low/undetectable in all samples on this panel (CTs>35). (Data not shown.)

Panel 4D Summary: Ag2934 Expression of the NOV24a gene is low/undetectable in all samples on this panel (CTs>35). (Data not shown.) The amp plot indicates that there is a high probability of a probe failure.

NOV25

Expression of gene NOV25 was assessed using the primer-probe sets Ag2935 and Ag3039, described in Tables WA and WB. Results of the RTQ-PCR runs are shown in Tables WC, WD and WE.

TABLE WA
Probe Name Ag2935

| | | | Start | |
|---|---|---|---|---|
| Primers | Sequence | Length | Position | SEQ ID NO: |
| Forward | 5′-ctggactctacctcggaaactt-3′ | 22 | 88 | 1075 |
| Probe | TET-5′-ctgggccgaaataagatcacacacat-3′-TAMRA | 26 | 135 | 1076 |
| Reverse | 5′-gggtgactcatggatagagatg-3′ | 22 | 161 | 1077 |

TABLE WB
Probe Name Ag3039

| | | | Start | |
|---|---|---|---|---|
| Primers | Sequence | Length | Position | SEQ ID NO: |
| Forward | 5′-gccgaaataagatcacacacat-3′ | 22 | 139 | 1078 |
| Probe | TET-5′-tctatccatgagtcaccccagcctct-3′-TAMRA | 26 | 165 | 1079 |
| Reverse | 5′-atgcgaaggtaggtgatatcct-3′ | 22 | 196 | 1080 |

TABLE WC
CNS_neurodegeneration_v1.0

| | Rel. Exp. (%) | Rel. Exp. (%) | | Rel. Exp. (%) | Rel. Exp. (%) |
|---|---|---|---|---|---|
| Tissue | Ag2935, Run | Ag3039, Run | Tissue | Ag2935, Run | Ag3039, Run |
| Name | 209777519 | 211012103 | Name | 209777519 | 211012103 |
| AD 1 Hippo | 34.2 | 18.4 | Control | 11.9 | 8.2 |
| | | | (Path) 3 | | |
| | | | Temporal | | |
| | | | Ctx | | |
| AD 2 Hippo | 59.5 | 48.0 | Control | 33.7 | 36.3 |
| | | | (Path) 4 | | |
| | | | Temporal | | |
| | | | Ctx | | |
| AD 3 Hippo | 15.3 | 9.8 | AD 1 | 24.3 | 9.5 |
| | | | Occipital | | |
| | | | Ctx | | |
| AD 4 Hippo | 18.7 | 13.6 | AD 2 | 0.0 | 0.0 |
| | | | Occipital | | |
| | | | Ctx | | |
| | | | (Missing) | | |
| AD 5 Hippo | 90.8 | 70.2 | AD 3 | 12.9 | 6.3 |
| | | | Occipital | | |
| | | | Ctx | | |
| AD 6 Hippo | 50.7 | 69.3 | AD 4 | 32.8 | 20.9 |
| | | | Occipital | | |
| | | | Ctx | | |
| Control 2 | 36.3 | 25.5 | AD 5 | 66.9 | 18.3 |
| Hippo | | | Occipital | | |
| | | | Ctx | | |
| Control 4 | 33.4 | 24.0 | AD 6 | 28.9 | 43.2 |
| Hippo | | | Occipital | | |
| | | | Ctx | | |
| Control | 7.2 | 7.6 | Control 1 | 7.6 | 6.0 |
| (Path) 3 | | | Occipital | | |
| Hippo | | | Ctx | | |
| AD 1 | 20.7 | 24.3 | Control 2 | 90.8 | 57.0 |
| Temporal | | | Occipital | | |
| Ctx | | | Ctx | | |
| AD 2 | 53.2 | 36.9 | Control 3 | 24.7 | 18.7 |
| Temporal | | | Occipital | | |
| Ctx | | | Ctx | | |
| AD 3 | 5.8 | 4.7 | Control 4 | 17.7 | 13.9 |
| Temporal | | | Occipital | | |
| Ctx | | | Ctx | | |
| AD 4 | 35.8 | 24.5 | Control | 93.3 | 74.2 |
| Temporal | | | (Path) 1 | | |
| Ctx | | | Occipital | | |
| | | | Ctx | | |
| AD 5 Inf | 100.0 | 100.0 | Control | 19.1 | 14.8 |
| Temporal | | | (Path) 2 | | |
| Ctx | | | Occipital | | |
| | | | Ctx | | |
| AD 5 Sup | 81.2 | 62.9 | Control | 8.6 | 4.3 |
| Temporal | | | (Path) 3 | | |
| Ctx | | | Occipital | | |
| | | | Ctx | | |
| AD 6 Inf | 62.4 | 58.2 | Control | 27.9 | 25.2 |
| Temporal | | | (Path) 4 | | |
| Ctx | | | Occipital | | |
| | | | Ctx | | |
| AD 6 Sup | 50.3 | 49.3 | Control 1 | 16.2 | 15.9 |
| Temporal | | | Parietal Ctx | | |
| Ctx | | | | | |
| Control 1 | 14.5 | 11.6 | Control 2 | 68.3 | 58.2 |
| Temporal | | | Parietal Ctx | | |
| Ctx | | | | | |
| Control 2 | 51.4 | 34.4 | Control 3 | 31.0 | 32.1 |
| Temporal | | | Parietal Ctx | | |
| Ctx | | | | | |
| Control 3 | 33.0 | 20.0 | Control | 69.7 | 66.9 |
| Temporal | | | (Path) 1 | | |
| Ctx | | | Parietal Ctx | | |
| Control 3 | 19.6 | 20.7 | Control | 31.2 | 39.0 |
| Temporal | | | (Path) 2 | | |
| Ctx | | | Parietal Ctx | | |
| Control | 55.1 | 44.4 | Control | 3.1 | 4.6 |
| (Path) 1 | | | (Path) 3 | | |
| Temporal | | | Parietal Ctx | | |
| Ctx | | | | | |
| Control | 56.3 | 30.4 | Control | 61.6 | 35.8 |
| (Path) 2 | | | (Path) 4 | | |
| Temporal | | | Parietal Ctx | | |
| Ctx | | | | | |

TABLE WD
Panel 1.3D

| | Rel. Exp. (%) | Rel. Exp. (%) | | Rel. Exp. (%) | Rel. Exp. (%) |
|---|---|---|---|---|---|
| | Ag2935, Run | Ag3039, Run | | Ag2935, Run | Ag3039, Run |
| Tissue Name | 167646849 | 167961816 | Tissue Name | 167646849 | 167961816 |
| Liver | 1.4 | 1.7 | Kidney (fetal) | 49.3 | 38.2 |
| adenocarcinoma | | | | | |
| Pancreas | 1.2 | 2.0 | Renal ca. 786-0 | 0.0 | 0.0 |
| Pancreatic ca. | 0.0 | 0.0 | Renal ca. | 1.7 | 0.4 |
| CAPAN 2 | | | A498 | | |
| Adrenal gland | 3.1 | 1.2 | Renal ca. RXF | 0.0 | 0.1 |
| | | | 393 | | |
| Thyroid | 11.7 | 6.0 | Renal ca. | 5.7 | 1.5 |
| | | | ACHN | | |
| Salivary gland | 1.3 | 0.8 | Renal ca. UO- | 0.0 | 0.0 |
| | | | 31 | | |
| Pituitary gland | 6.3 | 3.0 | Renal ca. TK- | 0.0 | 0.0 |
| | | | 10 | | |
| Brain (fetal) | 10.0 | 7.4 | Liver | 0.7 | 0.3 |
| Brain (whole) | 12.8 | 7.7 | Liver (fetal) | 1.8 | 0.7 |
| Brain (amygdala) | 6.9 | 6.0 | Liver ca. | 1.0 | 0.1 |
| | | | (hepatoblast) | | |
| | | | HepG2 | | |
| Brain (cerebellum) | 9.3 | 5.9 | Lung | 1.3 | 1.3 |
| Brain | 12.1 | 3.9 | Lung (fetal) | 6.5 | 3.0 |
| (hippocampus) | | | | | |
| Brain (substantia | 24.0 | 16.0 | Lung ca. | 0.3 | 0.0 |
| nigra) | | | (small cell) | | |
| | | | LX-1 | | |
| Brain (thalamus) | 5.1 | 3.8 | Lung ca. | 2.8 | 1.5 |
| | | | (small cell) | | |
| | | | NCI-H69 | | |
| Cerebral Cortex | 15.6 | 10.8 | Lung ca. | 7.2 | 3.7 |
| | | | (s.cell var.) | | |
| | | | SHP-77 | | |
| Spinal cord | 37.4 | 15.2 | Lung ca. (large | 0.0 | 0.1 |
| | | | cell)NCI-H460 | | |
| glio/astro U87-MG | 0.0 | 0.2 | Lung ca. (non- | 2.4 | 1.6 |
| | | | sm. cell) A549 | | |
| glio/astro U-118- | 0.4 | 0.1 | Lung ca. (non- | 4.0 | 0.4 |
| MG | | | s.cell) NCI- | | |
| | | | H23 | | |
| astrocytoma | 3.5 | 0.6 | Lung ca. (non- | 1.1 | 0.0 |
| SW1783 | | | s.cell) HOP-62 | | |
| neuro*; met SK-N- | 0.8 | 0.3 | Lung ca. (non- | 3.9 | 1.4 |
| AS | | | s.cl) NCI- | | |
| | | | H522 | | |
| astrocytoma SF- | 3.0 | 1.5 | Lung ca. | 2.7 | 0.7 |
| 539 | | | (squam.) SW | | |
| | | | 900 | | |
| astrocytoma SNB- | 2.5 | 1.2 | Lung ca. | 12.2 | 5.7 |
| 75 | | | (squam.) NCI- | | |
| | | | H596 | | |
| glioma SNB-19 | 2.7 | 2.1 | Mammary | 3.9 | 2.4 |
| | | | gland | | |
| glioma U251 | 1.0 | 0.3 | Breast ca.* | 0.7 | 0.7 |
| | | | (pl.ef) MCF-7 | | |
| glioma SF-295 | 2.9 | 2.3 | Breast ca.* | 0.4 | 0.1 |
| | | | (pl.ef) MDA- | | |
| | | | MB-231 | | |
| Heart (fetal) | 24.0 | 14.5 | Breast ca.* | 23.3 | 13.5 |
| | | | (pl.ef) T47D | | |
| Heart | 5.6 | 3.4 | Breast ca. BT- | 0.5 | 1.2 |
| | | | 549 | | |
| Skeletal muscle | 11.3 | 5.1 | Breast ca. | 6.2 | 6.4 |
| (fetal) | | | MDA-N | | |
| Skeletal muscle | 1.1 | 0.0 | Ovary | 11.4 | 3.9 |
| Bone marrow | 0.0 | 0.4 | Ovarian ca. | 1.0 | 0.3 |
| | | | OVCAR-3 | | |
| Thymus | 1.4 | 0.2 | Ovarian ca. | 21.3 | 13.8 |
| | | | OVCAR-4 | | |
| Spleen | 3.5 | 3.1 | Ovarian ca. | 5.6 | 1.5 |
| | | | OVCAR-5 | | |
| Lymph node | 0.7 | 0.9 | Ovarian ca. | 0.1 | 0.0 |
| | | | OVCAR-8 | | |
| Colorectal | 1.5 | 0.4 | Ovarian ca. | 0.0 | 0.0 |
| | | | IGROV-1 | | |
| Stomach | 2.5 | 0.6 | Ovarian ca.* | 0.4 | 0.4 |
| | | | (ascites) SK- | | |
| | | | OV-3 | | |
| Small intestine | 3.4 | 1.0 | Uterus | 3.0 | 2.2 |
| Colon ca. SW480 | 8.8 | 6.7 | Placenta | 0.6 | 0.0 |
| Colon ca.* | 0.0 | 0.0 | Prostate | 3.1 | 2.7 |
| SW620(SW480 | | | | | |
| met) | | | | | |
| Colon ca. HT29 | 0.0 | 0.0 | Prostate ca.* | 0.0 | 0.0 |
| | | | (bone met)PC-3 | | |
| Colon ca. HCT- | 0.0 | 0.0 | Testis | 100.0 | 100.0 |
| 116 | | | | | |
| Colon ca. CaCo-2 | 23.0 | 12.2 | Melanoma | 0.0 | 0.1 |
| | | | Hs688(A).T | | |
| Colon ca. | 1.3 | 1.2 | Melanoma* | 0.0 | 0.0 |
| tissue(ODO3866) | | | (met) | | |
| | | | Hs688(B).T | | |
| Colon ca. HCC- | 6.4 | 3.0 | Melanoma | 12.0 | 5.2 |
| 2998 | | | UACC-62 | | |
| Gastric ca.* (liver | 2.7 | 1.2 | Melanoma | 2.7 | 0.8 |
| met) NCI-N87 | | | M14 | | |
| Bladder | 3.1 | 1.8 | Melanoma | 0.0 | 0.0 |
| | | | LOX IMVI | | |
| Trachea | 1.4 | 0.6 | Melanoma* | 6.5 | 2.0 |
| | | | (met) SK- | | |
| | | | MEL-5 | | |
| Kidney | 84.7 | 41.8 | Adipose | 5.0 | 0.6 |

TABLE WE
Panel 4D

| | Rel. | Rel. | | Rel. | Rel. |
|---|---|---|---|---|---|
| | Exp. (%) | Exp. (%) | | Exp. (%) | Exp. (%) |
| | Ag2935, | Ag3039, | | Ag2935, | Ag3039, |
| | Run | Run | | Run | Run |
| Tissue Name | 164403313 | 162427949 | Tissue Name | 164403313 | 162427949 |
| Secondary Th1 act | 0.0 | 0.0 | HUVEC IL-1beta | 0.2 | 0.0 |
| Secondary Th2 act | 0.0 | 0.0 | HUVEC IFN | 0.2 | 0.3 |
| | | | gamma | | |
| Secondary Tr1 act | 0.0 | 0.0 | HUVEC TNF | 0.0 | 0.0 |
| | | | alpha + IFN | | |
| | | | gamma | | |
| Secondary Th1 rest | 0.0 | 0.0 | HUVEC TNF | 0.0 | 0.0 |
| | | | alpha + IL4 | | |
| Secondary Th2 rest | 0.0 | 0.0 | HUVEC IL-11 | 0.0 | 0.3 |
| Secondary Tr1 rest | 0.0 | 0.0 | Lung | 0.0 | 1.3 |
| | | | Microvascular EC | | |
| | | | none | | |
| Primary Th1 act | 0.0 | 0.0 | Lung | 0.0 | 0.0 |
| | | | Microvascular EC | | |
| | | | TNF alpha + IL- | | |
| | | | 1beta | | |
| Primary Th2 act | 0.0 | 0.0 | Microvascular | 0.3 | 0.3 |
| | | | Dermal EC none | | |
| Primary Tr1 act | 0.0 | 0.0 | Microsvasular | 0.3 | 0.4 |
| | | | Dermal EC | | |
| | | | TNF alpha + IL- | | |
| | | | 1beta | | |
| Primary Th1 rest | 0.0 | 0.0 | Bronchial | 1.7 | 1.4 |
| | | | epithelium | | |
| | | | TNF alpha + | | |
| | | | IL1beta | | |
| Primary Th2 rest | 0.0 | 0.0 | Small airway | 0.2 | 1.1 |
| | | | epithelium none | | |
| Primary Tr1 rest | 0.0 | 0.0 | Small airway | 0.3 | 0.3 |
| | | | epithelium | | |
| | | | TNF alpha + IL- | | |
| | | | 1beta | | |
| CD45RA CD4 | 0.4 | 0.0 | Coronery artery | 0.2 | 0.0 |
| lymphocyte act | | | SMC rest | | |
| CD45RO CD4 | 0.0 | 0.0 | Coronery artery | 0.0 | 0.0 |
| lymphocyte act | | | SMC TNF alpha + | | |
| | | | IL-1beta | | |
| CD8 lymphocyte | 0.1 | 0.0 | Astrocytes rest | 1.9 | 4.0 |
| act | | | | | |
| Secondary CD8 | 0.0 | 0.0 | Astrocytes | 2.4 | 2.3 |
| lymphocyte rest | | | TNF alpha + IL- | | |
| | | | 1beta | | |
| Secondary CD8 | 0.0 | 0.0 | KU-812 | 0.0 | 0.0 |
| lymphocyte act | | | (Basophil) rest | | |
| CD4 lymphocyte | 0.0 | 0.0 | KU-812 | 0.0 | 0.0 |
| none | | | (Basophil) | | |
| | | | PMA/ionomycin | | |
| 2ry | 0.0 | 0.0 | CCD1106 | 0.8 | 0.4 |
| Th1/Th2/Tr1_anti- | | | (Keratinocytes) | | |
| CD95 CH11 | | | none | | |
| LAK cells rest | 0.6 | 0.1 | CCD1106 | 0.5 | 0.4 |
| | | | (Keratinocytes) | | |
| | | | TNF alpha + IL- | | |
| | | | 1beta | | |
| LAK cells IL-2 | 0.0 | 0.4 | Liver cirrhosis | 0.0 | 0.7 |
| LAK cells IL-2 + IL- | 0.3 | 0.0 | Lupus kidney | 4.0 | 5.2 |
| 12 | | | | | |
| LAK cells IL- | 0.0 | 0.4 | NCI-H292 none | 1.4 | 3.5 |
| 2 + IFN gamma | | | | | |
| LAK cells IL-2 + | 0.8 | 0.8 | NCI-H292 IL-4 | 1.2 | 0.9 |
| IL-18 | | | | | |
| LAK cells | 0.3 | 0.0 | NCI-H292 IL-9 | 1.6 | 1.8 |
| PMA/ionomycin | | | | | |
| NK Cells IL-2 rest | 0.0 | 0.0 | NCI-H292 IL-13 | 2.1 | 1.2 |
| Two Way MLR 3 | 0.0 | 0.0 | NCI-H292 IFN | 2.3 | 3.6 |
| day | | | gamma | | |
| Two Way MLR 5 | 0.0 | 0.0 | HPAEC none | 0.0 | 0.0 |
| day | | | | | |
| Two Way MLR 7 | 0.0 | 0.0 | HPAEC TNF | 0.0 | 0.0 |
| day | | | alpha + IL-1beta | | |
| PBMC rest | 0.0 | 0.3 | Lung fibroblast | 0.0 | 0.0 |
| | | | none | | |
| PBMC PWM | 0.2 | 2.5 | Lung fibroblast | 0.0 | 0.0 |
| | | | TNF alpha + IL-1 | | |
| | | | beta | | |
| PBMC PHA-L | 0.4 | 1.6 | Lung fibroblast | 0.0 | 0.0 |
| | | | IL-4 | | |
| Ramos (B cell) | 0.0 | 0.0 | Lung fibroblast | 0.0 | 0.0 |
| none | | | IL-9 | | |
| Ramos (B cell) | 0.0 | 0.0 | Lung fibroblast | 0.0 | 0.5 |
| ionomycin | | | IL-13 | | |
| B lymphocytes | 3.0 | 5.5 | Lung fibroblast | 0.0 | 0.0 |
| PWM | | | IFN gamma | | |
| B lymphocytes | 1.0 | 1.3 | Dermal fibroblast | 0.0 | 0.0 |
| CD40L and IL-4 | | | CCD1070 rest | | |
| EOL-1 dbcAMP | 0.5 | 0.6 | Dermal fibroblast | 0.0 | 0.0 |
| | | | CCD1070 TNF | | |
| | | | alpha | | |
| EOL-1 dbcAMP | 0.3 | 0.0 | Dermal fibroblast | 0.0 | 0.0 |
| PMA/ionomycin | | | CCD1070 IL-1 | | |
| | | | beta | | |
| Dendritic cells | 0.0 | 0.0 | Dermal fibroblast | 0.0 | 0.0 |
| none | | | IFN gamma | | |
| Dendritic cells LPS | 0.1 | 0.4 | Dermal fibroblast | 0.0 | 0.0 |
| | | | IL-4 | | |
| Dendritic cells anti- | 0.3 | 0.0 | IBD Colitis 2 | 0.0 | 0.0 |
| CD40 | | | | | |
| Monocytes rest | 0.0 | 0.0 | IBD Crohn's | 0.2 | 1.1 |
| Monocytes LPS | 0.0 | 0.4 | Colon | 2.1 | 2.5 |
| Macrophages rest | 1.1 | 1.0 | Lung | 2.1 | 5.1 |
| Macrophages LPS | 0.3 | 0.4 | Thymus | 100.0 | 100.0 |
| HUVEC none | 0.0 | 0.6 | Kidney | 3.7 | 3.1 |
| HUVEC starved | 0.0 | 0.0 | | | |

CNS_neurodegeneration_v1.0 Summary: Ag2935/Ag3039 No differential expression of the NOV25 gene is detected between the postmortem brains of Alzheimer's diseased patients and those of non-demented controls. However, this panel confirms the expression of this gene in the CNS. Please see panel 1.3D for a discussion of utility of this gene in the central nervous system.

Panel 1.3D Summary: Ag2935/Ag3039 The expression of the NOV25 gene was assessed in two independent runs with good concordance between runs. Highest expression is seen in the testis (CTs=29). In addition, expression of this gene is extremely low in renal and brain cancer cell lines but is expressed in the normal brain and kidney tissues on this sample. Therefore, this gene may be used as a diagnostic marker for brain and kidney cancer and prostate tissue. Furthermore, therapeutic modulation of the expression or function of this gene may be effective in the treatment of brain and renal cancers.

This gene encodes a novel protein phosphatase expressed at moderate to low levels in the CNS that may therefore be a small molecule target for the treatment of neurologic diseases.

In addition, this gene is expressed at low levels in metabolic tissues including pancreas, adrenal, thyroid, pituitary, adult and fetal heart, and adipose. This novel protein phosphatase may be a small molecule target for the treatment of metabolic and endocrine disease, including obesity and Types 1 and 2 diabetes. This gene is also differentially expressed in fetal skeletal muscle (CT values=32-33) when compared to expression in adult skeletal muscle (CT values=35-40). Therefore, expression of this gene may also be useful for the differentiation of adult and fetal skeletal muscle.

Panel 4D Summary: Ag2935/Ag3039 Expression of the NOV25 gene is highest and almost exclusive to the thymus (CTs=29-30). Expression of this gene could be used to distinguish thymus from the other samples on this panel. The putative phosphatase encoded by this gene may play an important role in T cell development. Small molecule therapeutics designed against the protein encoded by this gene could therefore be utilized to modulate immune function (T cell development) and be important for organ transplant, AIDS treatment or post chemotherapy immune reconstitution.

NOV26a and NOV26b

Expression of gene NOV26a and variant NOV26b was assessed using the primer-probe set Ag2936, described in Table XA.

TABLE XA
Probe Name Ag2936

| | | | Start | |
|---|---|---|---|---|
| Primers | Sequence | Length | Position | SEQ ID NO: |
| Forward | 5′-gttcctgctgtctggactttt-3′ | 21 | 1 | 1081 |
| Probe | TET-5′-cccactgagacgcagctgtattctgt-3′-TAMRA | 26 | 27 | 1082 |
| Reverse | 5′-tcgccaaatcatatttcacact-3′ | 22 | 57 | 1083 |

CNS_neurodegeneration_v1.0 Summary: Ag2936 Expression of the NOV26a gene is low/undetectable in all samples on this panel (CTs>35). (Data not shown.) The amp plot indicates that there is a high probability of a probe failure.

Panel 1.3D Summary: Ag2936 Expression of the NOV26a gene is low/undetectable in all samples on this panel (CTs>35). (Data not shown.) The amp plot indicates that there is a high probability of a probe failure.

Panel 4D Summary: Ag2936 Expression of the NOV26a gene is low/undetectable in all samples on this panel (CTs>35). (Data not shown.) The amp plot indicates that there is a high probability of a probe failure.

NOV24a and NOV24b

Expression of gene NOV24a and variant NOV24b was assessed using the primer-probe set Ag2966, described in Table YA. Results of the RTQ-PCR runs are shown in Tables YB and YC.

TABLE YA
Probe Name Ag2966

| | | | Start | |
|---|---|---|---|---|
| Primers | Sequence | Length | Position | SEQ ID NO: |
| Forward | 5′-agcgggcaactcttcatcta-3′ | 20 | 1356 | 1084 |
| Probe | TET-5′-atgcagtcagtgaccagctccctg-3′-TAMRA | 24 | 1377 | 1085 |
| Reverse | 5′-caggacaaagactgcagtcact-3′ | 22 | 1409 | 1086 |

TABLE YB
Panel 1.3D

| | Rel. Exp. (%) | Rel. Exp. (%) | | Rel. Exp. (%) | Rel. Exp. (%) |
|---|---|---|---|---|---|
| | Ag2966, Run | Ag2966, Run | | Ag2966, Run | Ag2966, Run |
| Tissue Name | 160658385 | 165701959 | Tissue Name | 160658385 | 165701959 |
| Liver | 0.0 | 0.0 | Kidney (fetal) | 1.0 | 0.0 |
| adenocarcinoma | | | | | |
| Pancreas | 0.2 | 1.3 | Renal ca. 786-0 | 0.2 | 0.0 |
| Pancreatic ca. | 0.0 | 0.0 | Renal ca. | 0.0 | 0.3 |
| CAPAN 2 | | | A498 | | |
| Adrenal gland | 0.2 | 0.0 | Renal ca. RXF | 0.0 | 0.5 |
| | | | 393 | | |
| Thyroid | 0.3 | 0.0 | Renal ca. | 0.3 | 0.0 |
| | | | ACHN | | |
| Salivary gland | 0.1 | 0.4 | Renal ca. UO- | 0.0 | 0.0 |
| | | | 31 | | |
| Pituitary gland | 0.0 | 0.0 | Renal ca. TK- | 0.0 | 0.0 |
| | | | 10 | | |
| Brain (fetal) | 0.0 | 0.0 | Liver | 0.6 | 0.9 |
| Brain (whole) | 0.0 | 0.3 | Liver (fetal) | 0.4 | 0.0 |
| Brain (amygdala) | 0.0 | 0.0 | Liver ca. | 0.3 | 0.0 |
| | | | (hepatoblast) | | |
| | | | HepG2 | | |
| Brain (cerebellum) | 0.0 | 0.0 | Lung | 0.8 | 0.6 |
| Brain | 0.5 | 0.3 | Lung (fetal) | 1.4 | 1.4 |
| (hippocampus) | | | | | |
| Brain (substantia | 0.0 | 0.0 | Lung ca. | 0.6 | 0.0 |
| nigra) | | | (small cell) | | |
| | | | LX-1 | | |
| Brain (thalamus) | 0.0 | 0.5 | Lung ca. | 0.0 | 0.0 |
| | | | (small cell) | | |
| | | | NCI-H69 | | |
| Cerebral Cortex | 0.0 | 0.0 | Lung ca. | 0.3 | 0.4 |
| | | | (s.cell var.) | | |
| | | | SHP-77 | | |
| Spinal cord | 0.2 | 0.2 | Lung ca. (large | 0.2 | 0.0 |
| | | | cell)NCI-H460 | | |
| glio/astro U87-MG | 0.0 | 0.0 | Lung ca. (non- | 0.8 | 0.7 |
| | | | sm. cell) A549 | | |
| glio/astro U-118- | 0.0 | 0.0 | Lung ca. (non- | 0.0 | 0.0 |
| MG | | | s.cell) NCI- | | |
| | | | H23 | | |
| astrocytoma | 0.0 | 0.0 | Lung ca. (non- | 1.6 | 2.2 |
| SW1783 | | | s.cell) HOP-62 | | |
| neuro*; met SK-N- | 0.0 | 0.4 | Lung ca. (non- | 0.5 | 0.0 |
| AS | | | s.cl) NCI- | | |
| | | | H522 | | |
| astrocytoma SF- | 0.0 | 0.0 | Lung ca. | 0.0 | 0.0 |
| 539 | | | (squam.) SW | | |
| | | | 900 | | |
| astrocytoma SNB- | 0.0 | 0.0 | Lung ca. | 0.0 | 0.0 |
| 75 | | | (squam.) NCI- | | |
| | | | H596 | | |
| glioma SNB-19 | 0.0 | 0.4 | Mammary | 0.3 | 0.7 |
| | | | gland | | |
| glioma U251 | 0.0 | 0.4 | Breast ca.* | 0.2 | 0.0 |
| | | | (pl.ef) MCF-7 | | |
| glioma SF-295 | 0.0 | 0.0 | Breast ca.* | 0.0 | 0.0 |
| | | | (pl.ef) MDA- | | |
| | | | MB-231 | | |
| Heart (fetal) | 0.0 | 0.0 | Breast ca.* | 0.0 | 0.0 |
| | | | (pl.ef) T47D | | |
| Heart | 0.0 | 0.0 | Breast ca. BT- | 0.2 | 0.0 |
| | | | 549 | | |
| Skeletal muscle | 3.9 | 0.3 | Breast ca. | 0.0 | 0.0 |
| (fetal) | | | MDA-N | | |
| Skeletal muscle | 0.0 | 0.0 | Ovary | 0.5 | 0.0 |
| Bone marrow | 6.9 | 2.5 | Ovarian ca. | 0.0 | 0.0 |
| | | | OVCAR-3 | | |
| Thymus | 1.9 | 0.9 | Ovarian ca. | 0.0 | 0.4 |
| | | | OVCAR-4 | | |
| Spleen | 4.2 | 1.0 | Ovarian ca. | 0.3 | 0.0 |
| | | | OVCAR-5 | | |
| Lymph node | 3.0 | 5.4 | Ovarian ca. | 0.0 | 0.0 |
| | | | OVCAR-8 | | |
| Colorectal | 0.5 | 0.0 | Ovarian ca. | 0.0 | 0.0 |
| | | | IGROV-1 | | |
| Stomach | 0.9 | 0.0 | Ovarian ca.* | 0.0 | 0.0 |
| | | | (ascites) SK- | | |
| | | | OV-3 | | |
| Small intestine | 1.5 | 0.5 | Uterus | 0.0 | 0.5 |
| Colon ca. SW480 | 0.8 | 0.0 | Placenta | 0.0 | 0.0 |
| Colon ca.* | 0.0 | 0.0 | Prostate | 0.0 | 0.0 |
| SW620(SW480 | | | | | |
| met) | | | | | |
| Colon ca. HT29 | 0.0 | 0.0 | Prostate ca.* | 0.0 | 0.0 |
| | | | (bone met)PC-3 | | |
| Colon ca. HCT- | 0.0 | 0.0 | Testis | 1.6 | 0.8 |
| 116 | | | | | |
| Colon ca. CaCo-2 | 0.0 | 0.0 | Melanoma | 0.0 | 0.0 |
| | | | Hs688(A).T | | |
| Colon ca. | 0.3 | 0.3 | Melanoma* | 0.0 | 0.0 |
| tissue(ODO3866) | | | (met) | | |
| | | | Hs688(B).T | | |
| Colon ca. HCC- | 0.3 | 0.0 | Melanoma | 0.0 | 0.0 |
| 2998 | | | UACC-62 | | |
| Gastric ca.* (liver | 0.2 | 0.7 | Melanoma | 0.0 | 0.0 |
| met) NCI-N87 | | | M14 | | |
| Bladder | 0.2 | 0.3 | Melanoma | 0.0 | 0.0 |
| | | | LOX IMVI | | |
| Trachea | 0.6 | 0.0 | Melanoma* | 0.0 | 0.0 |
| | | | (met) SK- | | |
| | | | MEL-5 | | |
| Kidney | 100.0 | 100.0 | Adipose | 0.1 | 0.6 |

TABLE YC
Panel 4D

| | Rel. Exp. (%) | | Rel. Exp. (%) |
|---|---|---|---|
| | Ag2996, Run | | Ag2996, Run |
| Tissue Name | 160660646 | Tissue Name | 160660646 |
| Secondary Th1 act | 0.0 | HUVEC IL-1beta | 0.3 |
| Secondary Th2 act | 0.0 | HUVEC IFN gamma | 0.9 |
| Secondary Tr1 act | 0.7 | HUVEC TNF alpha + IFN | 0.3 |
| | | gamma | |
| Secondary Th1 rest | 0.2 | HUVEC TNF alpha + IL4 | 0.5 |
| Secondary Th2 rest | 1.6 | HUVEC IL-11 | 0.8 |
| Secondary Tr1 rest | 1.5 | Lung Microvascular EC | 2.0 |
| | | none | |
| Primary Th1 act | 0.3 | Lung Microvascular EC | 0.8 |
| | | TNF alpha + IL-1beta | |
| Primary Th2 act | 0.0 | Microvascular Dermal EC | 1.9 |
| | | none | |
| Primary Tr1 act | 0.3 | Microsvasular Dermal EC | 1.7 |
| | | TNF alpha + IL-1beta | |
| Primary Th1 rest | 4.8 | Bronchial epithelium | 0.0 |
| | | TNF alpha + IL1beta | |
| Primary Th2 rest | 2.1 | Small airway epithelium | 0.0 |
| | | none | |
| Primary Tr1 rest | 3.4 | Small airway epithelium | 0.3 |
| | | TNF alpha + IL-1beta | |
| CD45RA CD4 | 0.6 | Coronery artery SMC rest | 0.0 |
| lymphocyte act | | | |
| CD45RO CD4 | 1.1 | Coronery artery SMC | 0.3 |
| lymphocyte act | | TNF alpha + IL-1beta | |
| CD8 lymphocyte act | 0.3 | Astrocytes rest | 0.0 |
| Secondary CD8 | 1.3 | Astrocytes TNF alpha + | 0.3 |
| lymphocyte rest | | IL-1beta | |
| Secondary CD8 | 0.0 | KU-812 (Basophil) rest | 0.3 |
| lymphocyte act | | | |
| CD4 lymphocyte none | 1.7 | KU-812 (Basophil) | 0.0 |
| | | PMA/ionomycin | |
| 2ry Th1/Th2/Tr1_anti- | 0.8 | CCD1106 (Keratinocytes) | 0.1 |
| CD95 CH11 | | none | |
| LAK cells rest | 2.1 | CCD1106 (Keratinocytes) | 0.0 |
| | | TNF alpha + IL-1beta | |
| LAK cells IL-2 | 0.4 | Liver cirrhosis | 0.4 |
| LAK cells IL-2 + IL-12 | 0.3 | Lupus kidney | 1.0 |
| LAK cells IL-2 + IFN | 0.6 | NCI-H292 none | 0.6 |
| gamma | | | |
| LAK cells IL-2 + IL-18 | 0.6 | NCI-H292 IL-4 | 0.0 |
| LAK cells | 0.0 | NCI-H292 IL-9 | 0.0 |
| PMA/ionomycin | | | |
| NK Cells IL-2 rest | 0.6 | NCI-H292 IL-13 | 0.0 |
| Two Way MLR 3 day | 1.1 | NCI-H292 IFN gamma | 0.0 |
| Two Way MLR 5 day | 1.0 | HPAEC none | 0.6 |
| Two Way MLR 7 day | 0.5 | HPAEC TNF alpha + IL- | 0.0 |
| | | 1beta | |
| PBMC rest | 0.6 | Lung fibroblast none | 0.0 |
| PBMC PWM | 1.1 | Lung fibroblast TNF alpha + | 0.0 |
| | | IL-1beta | |
| PBMC PHA-L | 0.2 | Lung fibroblast IL-4 | 0.0 |
| Ramos (B cell) none | 1.7 | Lung fibroblast IL-9 | 0.0 |
| Ramos (B cell) | 1.1 | Lung fibroblast IL-13 | 0.0 |
| ionomycin | | | |
| B lymphocytes PWM | 1.4 | Lung fibroblast IFN | 0.2 |
| | | gamma | |
| B lymphocytes CD40L | 4.7 | Dermal fibroblast | 0.0 |
| and IL-4 | | CCD1070 rest | |
| EOL-1 dbcAMP | 0.0 | Dermal fibroblast | 1.4 |
| | | CCD1070 TNF alpha | |
| EOL-1 dbcAMP | 0.1 | Dermal fibroblast | 0.0 |
| PMA/ionomycin | | CCD1070 IL-1beta | |
| Dendritic cells none | 0.5 | Dermal fibroblast IFN | 0.0 |
| | | gamma | |
| Dendritic cells LPS | 0.0 | Dermal fibroblast IL-4 | 0.0 |
| Dendritic cells anti- | 0.5 | IBD Colitis 2 | 0.2 |
| CD40 | | | |
| Monocytes rest | 1.2 | IBD Crohn's | 0.0 |
| Monocytes LPS | 0.0 | Colon | 4.9 |
| Macrophages rest | 0.2 | Lung | 3.1 |
| Macrophages LPS | 0.0 | Thymus | 100.0 |
| HUVEC none | 0.5 | Kidney | 4.6 |
| HUVEC starved | 0.3 | | |

Panel 1.3D Summary: Ag2966 Two experiments both show that expression of the NOV24a gene, a sodium-glucose cotransporter homolog, is limited to the kidney (CTs=29). This restricted expression is in agreement with published data which has shown that secondary active transport of glucose in the kidney is mediated by sodium glucose cotransporter. (See ref. 1). Thus, expression of this gene could be used as a marker for kidney tissue. Furthermore, the protein product may be important for normal function of the kidney. Thus, therapeutic modulation of the expression or function of this protein may be useful in treating diseases that affect the kidney, including diabetes.

References:

Bissonnette P, Noel J, Coady M J, Lapointe J Y. Functional expression of tagged human Na+-glucose cotransporter in Xenopus laevis oocytes. J Physiol Oct. 15, 1999;520 Pt 2:359-71

1. High-affinity, secondary active transport of glucose in the intestine and kidney is mediated by an integral membrane protein named SGLT1 (sodium glucose cotransporter). Though basic properties of the transporter are now defined, many questions regarding the structure-function relationship of the protein, its biosynthesis and targeting remain unanswered. In order to better address these questions, we produced a functional hSGLT1 protein (from human) containing a reporter tag. 2. Six constructs, made from three tags (myc, haemaglutinin and poly-His) inserted at both the C- and N-terminal positions, were thus tested using the Xenopus oocyte expression system via electrophysiology and immunohistochemistry. Of these, only the hSGLT1 construct with the myc tag inserted at the N-terminal position proved to be of interest, all other constructs showing no or little transport activity. A systematic comparison of transport properties was therefore performed between the myc-tagged and the untagged hSGLT1 proteins. 3. On the basis of both steady-state (affinities for substrate (glucose) and inhibitor (phlorizin) as well as expression levels) and presteady-state parameters (transient currents) we conclude that the two proteins are functionally indistinguishable, at least under these criteria. Immunological detection confirmed the appropriate targeting of the tagged protein to the plasma membrane of the oocyte with the epitope located at the extracellular side. 4. The myc-tagged hSGLT1 was also successfully expressed in polarized MDCK cells. alpha-Methylglucose uptake studies on transfected cells showed an exclusively apical uptake pathway, thus indicating that the expressed protein was correctly targeted to the apical domain of the cell. 5. These comparative studies demonstrate that the myc epitope inserted at the N-terminus of hSGLT1 produces a fully functional protein while other epitopes of similar size inserted at either end of the protein inactivated the final protein.

PMID: 10523405

Panel 2D Summary: Ag2966 Expression of the NOV24a gene is predominantly limited to the kidney. This result is in agreement with the expression seen in Panel 1.3D.

Panel 3D Summary: Ag2966 Results from one experiment with the NOV24a gene are not included. The amp plot indicates that there were experimental difficulties with this run.

Panel 4D Summary: Ag2966 Expression of the NOV24a gene is predominantly found in normal tissue from thymus, lung, colon and kidney. This expression profile suggests that the protein product may be involved in glucose transport in these tissues. Therefore, therapeutic modulation of the expression or function of this protein may be useful in treating diseases that affect these organs.

NOV28

Expression of gene NOV28 was assessed using the primer-probe set Ag2891, described in Table ZA. Results of the RTQ-PCR runs are shown in Tables ZB, ZC, ZD and ZE.

TABLE ZA
Probe Name Ag2891

| | | | Start | |
|---|---|---|---|---|
| Primers | Sequence | Length | Position | SEQ ID NO: |
| Forward | 5′-ccggaaattaatctaccatcaa-3′ | 22 | 252 | 1087 |
| Probe | TET-5′-ccccgggtaacaactgtttcagatct-3′-TAMRA | 26 | 289 | 1088 |
| Reverse | 5′-gatggaaaagtccatgttggt-3′ | 21 | 325 | 1089 |

TABLE ZB
CNS_neurodegeneration_v1.0

| | Rel. Exp. (%) Ag2891, | | Rel. Exp. (%) Ag2891, |
|---|---|---|---|
| Tissue Name | Run 224116294 | Tissue Name | Run 224116294 |
| AD 1 Hippo | 13.9 | Control (Path) 3 | 43.2 |
| | | Temporal Ctx | |
| AD 2 Hippo | 27.0 | Control (Path) 4 | 20.9 |
| | | Temporal Ctx | |
| AD 3 Hippo | 36.1 | AD 1 Occipital Ctx | 6.4 |
| AD 4 Hippo | 16.5 | AD 2 Occipital Ctx | 0.0 |
| | | (Missing) | |
| AD 5 hippo | 35.1 | AD 3 Occipital Ctx | 19.1 |
| AD 6 Hippo | 29.9 | AD 4 Occipital Ctx | 51.1 |
| Control 2 Hippo | 6.0 | AD 5 Occipital Ctx | 0.0 |
| Control 4 Hippo | 34.4 | AD 6 Occipital Ctx | 11.0 |
| Control (Path) 3 | 17.4 | Control 1 Occipital | 20.6 |
| Hippo | | Ctx | |
| AD 1 Temporal Ctx | 76.3 | Control 2 Occipital | 18.2 |
| | | Ctx | |
| AD 2 Temporal Ctx | 23.3 | Control 3 Occipital | 0.0 |
| | | Ctx | |
| AD 3 Temporal Ctx | 0.0 | Control 4 Occipital | 17.3 |
| | | Ctx | |
| AD 4 Temporal Ctx | 34.6 | Control (Path) 1 | 65.1 |
| | | Occipital Ctx | |
| AD 5 Inf Temporal | 24.5 | Control (Path) 2 | 13.5 |
| Ctx | | Occipital Ctx | |
| AD 5 Sup Temporal | 58.2 | Control (Path) 3 | 0.0 |
| Ctx | | Occipital Ctx | |
| AD 6 Inf Temporal | 45.1 | Control (Path) 4 | 17.6 |
| Ctx | | Occipital Ctx | |
| AD 6 Sup Temporal | 95.9 | Control 1 Parietal | 0.0 |
| Ctx | | Ctx | |
| Control 1 Temporal | 5.2 | Control 2 Parietal | 19.2 |
| Ctx | | Ctx | |
| Control 2 Temporal | 0.0 | Control 3 Parietal | 37.9 |
| Ctx | | Ctx | |
| Control 3 Temporal | 12.4 | Control (Path) 1 | 100.0 |
| Ctx | | Parietal Ctx | |
| Control 4 Temporal | 21.6 | Control (Path) 2 | 25.9 |
| Ctx | | Parietal Ctx | |
| Control (Path) 1 | 65.1 | Control (Path) 3 | 0.0 |
| Temporal Ctx | | Parietal Ctx | |
| Control (Path) 2 | 55.9 | Control (Path) 4 | 11.2 |
| Temporal Ctx | | Parietal Ctx | |

TABLE ZC
Panel 1.3D

| | Rel. Exp.(%) | Rel. Exp.(%) | | Rel. Exp.(%) | Rel. Exp.(%) |
|---|---|---|---|---|---|
| | Ag2891, Run | Ag2891, Run | | Ag2891, Run | Ag2891, Run |
| Tissue Name | 160898914 | 165701349 | Tissue Name | 160898914 | 165701349 |
| Liver | 0.0 | 0.0 | Kidney (fetal) | 0.0 | 0.0 |
| adenocarcinoma | | | | | |
| Pancreas | 0.0 | 0.0 | Renal ca. 786-0 | 72.7 | 17.7 |
| Pancreatic ca. | 0.0 | 0.0 | Renal ca. | 0.0 | 0.0 |
| CAPAN 2 | | | A498 | | |
| Adrenal gland | 0.0 | 0.0 | Renal ca. RXF | 98.6 | 100.0 |
| | | | 393 | | |
| Thyroid | 0.0 | 0.0 | Renal ca. | 35.6 | 21.3 |
| | | | ACHN | | |
| Salivary gland | 0.0 | 0.0 | Renal ca. UO- | 43.5 | 17.9 |
| | | | 31 | | |
| Pituitary gland | 0.0 | 0.0 | Renal ca. TK- | 0.0 | 0.0 |
| | | | 10 | | |
| Brain (fetal) | 0.0 | 0.0 | Liver | 0.0 | 0.0 |
| Brain (whole) | 0.0 | 0.0 | Liver (fetal) | 0.0 | 0.0 |
| Brain (amygdala) | 18.4 | 0.0 | Liver ca. | 0.0 | 0.0 |
| | | | (hepatoblast) | | |
| | | | HepG2 | | |
| Brain (cerebellum) | 1.9 | 10.2 | Lung | 0.0 | 0.0 |
| Brain | 19.6 | 12.2 | Lung (fetal) | 0.0 | 0.0 |
| (hippocampus) | | | | | |
| Brain (substantia | 0.0 | 0.0 | Lung ca. | 0.0 | 0.0 |
| nigra) | | | (small cell) | | |
| | | | LX-1 | | |
| Brain (thalamus) | 0.0 | 0.0 | Lung ca. | 0.0 | 0.0 |
| | | | (small cell) | | |
| | | | NCI-H69 | | |
| Cerebral Cortex | 100.0 | 11.8 | Lung ca. | 0.0 | 12.4 |
| | | | (s.cell var.) | | |
| | | | SHP-77 | | |
| Spinal cord | 14.9 | 13.5 | Lung ca. (large | 0.0 | 0.0 |
| | | | cell)NCI-H460 | | |
| glio/astro U87-MG | 0.0 | 0.0 | Lung ca. (non- | 0.0 | 26.1 |
| | | | sm. cell) A549 | | |
| glio/astro U-118- | 0.0 | 0.0 | Lung ca. (non- | 16.3 | 0.0 |
| MG | | | s.cell) NCI- | | |
| | | | H23 | | |
| astrocytoma | 0.0 | 0.0 | Lung ca. (non- | 0.0 | 0.0 |
| SW1783 | | | s.cell) HOP-62 | | |
| neuro*; met SK-N- | 0.0 | 0.0 | Lung ca. (non- | 19.8 | 0.0 |
| AS | | | s.cl) NCI- | | |
| | | | H522 | | |
| astrocytoma SF- | 0.0 | 10.5 | Lung ca. | 0.0 | 0.0 |
| 539 | | | (squam.) SW | | |
| | | | 900 | | |
| astrocytoma SNB- | 0.0 | 0.0 | Lung ca. | 0.0 | 0.0 |
| 75 | | | (squam.) NCI- | | |
| | | | H596 | | |
| glioma SNB-19 | 0.0 | 0.0 | Mammary | 0.0 | 37.4 |
| | | | gland | | |
| glioma U251 | 14.4 | 0.0 | Breast ca.* | 0.0 | 0.0 |
| | | | (pl.ef) MCF-7 | | |
| glioma SF-295 | 0.0 | 0.0 | Breast ca.* | 0.0 | 0.0 |
| | | | (pl.ef) MDA- | | |
| | | | MB-231 | | |
| Heart (fetal) | 0.0 | 0.0 | Breast ca.* | 14.6 | 0.0 |
| | | | (pl.ef) T47D | | |
| Heart | 0.0 | 0.0 | Breast ca. BT- | 0.0 | 0.0 |
| | | | 549 | | |
| Skeletal muscle | 15.8 | 0.0 | Breast ca. | 0.0 | 0.0 |
| (fetal) | | | MDA-N | | |
| Skeletal muscle | 0.0 | 0.0 | Ovary | 58.6 | 0.0 |
| Bone marrow | 0.0 | 0.0 | Ovarian ca. | 0.0 | 4.6 |
| | | | OVCAR-3 | | |
| Thymus | 0.0 | 0.0 | Ovarian ca. | 0.0 | 0.0 |
| | | | OVCAR-4 | | |
| Spleen | 0.0 | 0.0 | Ovarian ca. | 0.0 | 0.0 |
| | | | OVCAR-5 | | |
| Lymph node | 0.0 | 0.0 | Ovarian ca. | 0.0 | 0.0 |
| | | | OVCAR-8 | | |
| Colorectal | 26.2 | 0.0 | Ovarian ca. | 0.0 | 0.0 |
| | | | IGROV-1 | | |
| Stomach | 0.0 | 0.0 | Ovarian ca.* | 0.0 | 0.0 |
| | | | (ascites) SK- | | |
| | | | OV-3 | | |
| Small intestine | 0.0 | 30.4 | Uterus | 0.0 | 0.0 |
| Colon ca. SW480 | 6.7 | 23.3 | Placenta | 0.0 | 0.0 |
| Colon ca.* | 0.0 | 0.0 | Prostate | 0.0 | 0.0 |
| SW620(SW480 | | | | | |
| met) | | | | | |
| Colon ca. HT29 | 0.0 | 0.0 | Prostate ca.* | 0.0 | 0.0 |
| | | | (bone met)PC-3 | | |
| Colon ca. HCT- | 0.0 | 0.0 | Testis | 92.0 | 71.2 |
| 116 | | | | | |
| Colon ca. CaCo-2 | 0.0 | 0.0 | Melanoma | 0.0 | 0.0 |
| | | | Hs688(A).T | | |
| Colon ca. | 0.0 | 0.0 | Melanoma* | 0.0 | 0.0 |
| tissue(ODO3866) | | | (met) | | |
| | | | Hs688(B).T | | |
| Colon ca. HCC- | 0.0 | 0.0 | Melanoma | 0.0 | 0.0 |
| 2998 | | | UACC-62 | | |
| Gastric ca.* (liver | 0.0 | 0.0 | Melanoma | 3.3 | 0.0 |
| met) NCI-N87 | | | M14 | | |
| Bladder | 0.0 | 0.0 | Melanoma | 0.0 | 0.0 |
| | | | LOX IMVI | | |
| Trachea | 0.0 | 0.0 | Melanoma* | 0.0 | 0.0 |
| | | | (met) SK- | | |
| | | | MEL-5 | | |
| Kidney | 0.0 | 0.0 | Adipose | 0.0 | 0.0 |

TABLE ZD
Panel 2D

| | Rel. Exp. (%) | | Rel. Exp. (%) |
|---|---|---|---|
| | Ag2891, Run | | Ag2891, Run |
| Tissue Name | 160899401 | Tissue Name | 160899401 |
| Normal Colon | 0.0 | Kidney Margin | 18.2 |
| | | 8120608 | |
| CC Well to Mod Diff | 15.6 | Kidney Cancer | 0.0 |
| (ODO3866) | | 8120613 | |
| CC Margin (ODO3866) | 0.0 | Kidney Margin | 37.1 |
| | | 8120614 | |
| CC Gr.2 rectosigmoid | 0.0 | Kidney Cancer | 100.0 |
| (ODO3868) | | 9010320 | |
| CC Margin (ODO3868) | 0.0 | Kidney Margin | 6.3 |
| | | 9010321 | |
| CC Mod Diff (ODO3920) | 0.0 | Normal Uterus | 0.0 |
| CC Margin (ODO3920) | 0.0 | Uterus Cancer 064011 | 0.0 |
| CC Gr.2 ascend colon | 0.0 | Normal Thyroid | 0.0 |
| (ODO3921) | | | |
| CC Margin (ODO3921) | 0.0 | Thyroid Cancer | 0.0 |
| | | 064010 | |
| CC from Partial | 0.0 | Thyroid Cancer | 20.6 |
| Hepatectomy (ODO4309) | | A302152 | |
| Mets | | | |
| Liver Margin (ODO4309) | 0.0 | Thyroid Margin | 0.0 |
| | | A302153 | |
| Colon mets to lung | 0.0 | Normal Breast | 0.0 |
| (OD04451-01) | | | |
| Lung Margin (OD04451- | 0.0 | Breast Cancer | 0.0 |
| 02) | | (OD04566) | |
| Normal Prostate 6546-1 | 0.0 | Breast Cancer | 0.0 |
| | | (OD04590-01) | |
| Prostate Cancer | 0.0 | Breast Cancer Mets | 0.0 |
| (OD04410) | | (OD04590-03) | |
| Prostate Margin | 17.2 | Breast Cancer | 0.0 |
| (OD04410) | | Metastasis | |
| | | (OD04655-05) | |
| Prostate Cancer | 0.0 | Breast Cancer 064006 | 0.0 |
| (OD04720-01) | | | |
| Prostate Margin | 0.0 | Breast Cancer 1024 | 0.0 |
| (OD04720-02) | | | |
| Normal Lung 061010 | 24.7 | Breast Cancer | 0.0 |
| | | 9100266 | |
| Lung Met to Muscle | 15.7 | Breast Margin | 0.0 |
| (ODO4286) | | 9100265 | |
| Muscle Margin | 0.0 | Breast Cancer | 0.0 |
| (ODO4286) | | A209073 | |
| Lung Malignant Cancer | 0.0 | Breast Margin | 0.0 |
| (OD03126) | | A2090734 | |
| Lung Margin (OD03126) | 0.0 | Normal Liver | 0.0 |
| Lung Cancer (OD04404) | 0.0 | Liver Cancer 064003 | 0.0 |
| Lung Margin (OD04404) | 34.4 | Liver Cancer 1025 | 8.7 |
| Lung Cancer (OD04565) | 0.0 | Liver Cancer 1026 | 36.6 |
| Lung Margin (OD04565) | 8.4 | Liver Cancer 6004-T | 8.0 |
| Lung Cancer (OD04237- | 0.0 | Liver Tissue 6004-N | 0.0 |
| 01) | | | |
| Lung Margin (OD04237- | 0.0 | Liver Cancer 6005-T | 8.2 |
| 02) | | | |
| Ocular Mel Met to Liver | 17.0 | Liver Tissue 6005-N | 0.0 |
| (ODO4310) | | | |
| Liver Margin (ODO4310) | 0.0 | Normal Bladder | 8.3 |
| Melanoma Mets to Lung | 0.0 | Bladder Cancer 1023 | 0.0 |
| (OD04321) | | | |
| Lung Margin (OD04321) | 45.7 | Bladder Cancer | 10.8 |
| | | A302173 | |
| Normal Kidney | 41.5 | Bladder Cancer | 8.2 |
| | | (OD04718-01) | |
| Kidney Ca, Nuclear grade | 28.3 | Bladder Normal | 0.0 |
| 2 (OD04338) | | Adjacent (OD04718- | |
| | | 03) | |
| Kidney Margin | 47.3 | Normal Ovary | 0.0 |
| (OD04338) | | | |
| Kidney Ca Nuclear grade | 62.9 | Ovarian Cancer | 9.1 |
| 1/2 (OD04339) | | 064008 | |
| Kidney Margin | 40.9 | Ovarian Cancer | 0.0 |
| (OD04339) | | (OD04768-07) | |
| Kidney Ca, Clear cell | 97.3 | Ovary Margin | 0.0 |
| type (OD04340) | | (OD04768-08) | |
| Kidney Margin | 17.4 | Normal Stomach | 35.1 |
| (OD04340) | | | |
| Kidney Ca, Nuclear grade | 0.0 | Gastric Cancer | 0.0 |
| 3 (OD04348) | | 9060358 | |
| Kidney Margin | 3.2 | Stomach Margin | 0.0 |
| (OD04348) | | 9060359 | |
| Kidney Cancer | 8.3 | Gastric Cancer | 0.0 |
| (OD04622-01) | | 9060395 | |
| Kidney Margin | 17.7 | Stomach Margin | 23.5 |
| (OD04622-03) | | 9060394 | |
| Kidney Cancer | 0.0 | Gastric Cancer | 8.1 |
| (OD04450-01) | | 9060397 | |
| Kidney Margin | 4.3 | Stomach Margin 9060396 | 0.0 |
| (OD04450-03) | | | |
| Kidney Cancer 8120607 | 10.6 | Gastric Cancer | 0.0 |
| | | 064005 | |

TABLE ZE
Panel 4D

| | Rel. Exp. (%) | | Rel. Exp. (%) |
|---|---|---|---|
| | Ag2891, Run | | Ag2891, Run |
| Tissue Name | 159632970 | Tissue Name | 159632970 |
| Secondary Th1 act | 0.0 | HUVECM IL-1beta | 0.0 |
| Secondary Th2 act | 0.0 | HUVEC IFN gamma | 0.0 |
| Secondary Tr1 act | 0.0 | HUVEC TNF alpha + IFN | 0.0 |
| | | gamma | |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC | 0.0 |
| | | none | |
| Primary Th1 act | 0.0 | Lung Microvascular EC | 0.0 |
| | | TNF alpha + IL-1beta | |
| Primary Th2 act | 0.0 | Microvascular Dermal EC | 0.0 |
| | | none | |
| Primary Tr1 act | 0.0 | Microsvasular Dermal EC | 0.0 |
| | | TNF alpha + IL-1beta | |
| Primary Th1 rest | 0.0 | Bronchial epithelium | 0.0 |
| | | TNF alpha + IL1beta | |
| Primary Th2 rest | 0.0 | Small airway epithelium | 0.0 |
| | | none | |
| Primary Tr1 rest | 0.0 | Small airway epithelium | 0.0 |
| | | TNF alpha + IL-1beta | |
| CD45RA CD4 | 0.0 | Coronery artery SMC rest | 0.0 |
| lymphocyte act | | | |
| CD45RO CD4 | 0.0 | Coronery artery SMC | 0.0 |
| lymphocyte act | | TNF alpha + IL-1beta | |
| CD8 lymphocyte act | 0.0 | Astrocytes rest | 0.0 |
| Secondary CD8 | 0.0 | Astrocytes TNF alpha + | 0.0 |
| lymphocyte rest | | IL-1beta | |
| Secondary CD8 | 3.5 | KU-812 (Basophil) rest | 0.0 |
| lymphocyte act | | | |
| CD4 lymphocyte none | 0.0 | KU-812 (Basophil) | 0.0 |
| | | PMA/ionomycin | |
| 2ry Th1/Th2/Tr1_anti- | 0.0 | CCD1106 (Keratinocytes) | 0.0 |
| CD95 CH11 | | none | |
| LAK cells rest | 0.0 | CCD1106 (Keratinocytes) | 0.0 |
| | | TNF alpha + IL-1beta | |
| LAK cells IL-2 | 0.0 | Liver cirrhosis | 41.8 |
| LAK cells IL-2 + IL-12 | 0.0 | Lupus kidney | 0.0 |
| LAK cells IL-2 + IFN | 0.0 | NCI-H292 none | 0.0 |
| gamma | | | |
| LAK cells IL-2 + IL-18 | 0.0 | NCI-H292 IL-4 | 1.5 |
| LAK cells | 0.0 | NCI-H292 IL-9 | 19.5 |
| PMA/ionomycin | | | |
| NK Cells IL-2 rest | 0.0 | NCI-H292 IL-13 | 0.0 |
| Two Way MLR 3 day | 0.0 | NCI-H292 IFN gamma | 0.0 |
| Two Way MLR 5 day | 0.0 | HPAEC none | 0.0 |
| Two Way MLR 7 day | 0.0 | HPAEC TNF alpha + IL- | 0.0 |
| | | 1beta | |
| PBMC rest | 0.0 | Lung fibroblast none | 0.0 |
| PBMC PWM | 0.0 | Lung fibroblast TNF alpha + | 0.0 |
| | | IL-1beta | |
| PBMC PHA-L | 0.0 | Lung fibroblast IL-4 | 0.0 |
| Ramos (B cell) none | 0.0 | Lung fibroblast IL-9 | 0.0 |
| Ramos (B cell) | 0.0 | Lung fibroblast IL-13 | 0.0 |
| ionomycin | | | |
| B lymphocytes PWM | 0.0 | Lung fibroblast IFN | 0.0 |
| | | gamma | |
| B lymphocytes CD40L | 0.0 | Dermal fibroblast | 0.0 |
| and IL-4 | | CCD1070 rest | |
| EOL-1 dbcAMP | 7.3 | Dermal fibroblast | 0.0 |
| | | CCD1070 TNF alpha | |
| EOL-1 dbcAMP | 0.0 | Dermal fibroblast | 0.0 |
| PMA/ionomycin | | CCD1070 IL-1beta | |
| Dendritic cells none | 0.0 | Dermal fibroblast IFN | 0.0 |
| | | gamma | |
| Dendritic cells LPS | 2.2 | Dermal fibroblast IL-4 | 0.0 |
| Dendritic cells anti- | 0.0 | IBD Colitis 2 | 4.5 |
| CD40 | | | |
| Monocytes rest | 0.0 | IBD Crohn's | 0.0 |
| Monocytes LPS | 0.0 | Colon | 100.0 |
| Macrophages rest | 0.0 | Lung | 32.3 |
| Macrophages LPS | 1.3 | Thymus | 40.3 |
| HUVEC none | 0.0 | Kidney | 0.0 |
| HUVEC starved | 0.0 | | |

CNS_neurodegeneration_v1.0 Summary: Ag2891 The NOV28 gene is expressed at very low levels in the CNS. No differential expression is detected in the postmortem brains of Alzheimer's patients when compared with non-demented controls. The widespread expression in the brain however suggests that this gene may be of utility in the treatment of neurological diseases.

Panel 1.3D Summary: Ag2891 Two experiments with the same probe and primer set produce results that are in excellent agreement, with highest expression of the NOV28 gene in a lung cancer cell line and the brain (CTs=33-34). Significant expression is also seen in the testis and a cluster of lung cancer cell lines. Thus, expression of this gene could be used to differentiate these samples from other samples on this panel, and as a marker of testis tissue and lung cancer.

Panel 2D Summary: Ag2891 Expression of the NOV28 gene is limited to samples derived from kidney cancer (CTs=33-34). Thus, expression of this gene could be used to differentiate between these samples and other samples on this panel and as a marker to detect the presence of kidney cancer. Furthermore, therapeutic modulation of the expression or function of this gene may be effective in the treatment of kidney cancer.

Panel 3D Summary: Ag2891 Expression of the NOV28 gene is low/undetectable in all samples on this panel (CTs>35). (Data not shown.)

Panel 4D Summary: Ag2891 The NOV28 transcript is expressed at low but significant levels in the colon and thymus (CTs=33-35). Thus, the transcript or the protein it encodes could be used for detection of these tissues. The protein encoded by this transcript may also play an important role in the normal homeostasis of these tissues. Therefore, therapeutics designed with the protein encoded by this transcript could be important for modulating T cell development in the thymus or maintaining or restoring normal function to these organs during inflammation due to inflammatory bowel disease in the colon.

NOV29a

Expression of gene NOV29a was assessed using the primer-probe set Ag2892, described in Table AAA. Results of the RTQ-PCR runs are shown in Tables AAB, AAC, AAD and AAE.

TABLE AAA
Probe Name Ag2892

| | | | Start | |
|---|---|---|---|---|
| Primers | Sequence | Length | Position | SEQ ID NO: |
| Forward | 5′-ggcagctcgaaactttgac-3′ | 9 | 171 | 1090 |
| Probe | TET-5′-cagaaatccgaagacatgctccgaag-3′-TAMRA | 26 | 193 | 1091 |
| Reverse | 5′-gacaatgttgtccaggtcttgt-3′ | 22 | 240 | 1092 |

TABLE AAB
Panel 1.3D

| | Rel. Exp. (%) Ag2892, | | Rel. Exp. (%) Ag2892, |
|---|---|---|---|
| Tissue Name | Run 165721697 | Tissue Name | Run 165721697 |
| Liver adenocarcinoma | 4.6 | Kidney (fetal) | 4.4 |
| Pancreas | 0.7 | Renal ca. 786-0 | 3.6 |
| Pancreatic ca. CAPAN 2 | 0.0 | Renal ca. A498 | 0.0 |
| Adrenal gland | 4.2 | Renal ca. RXF 393 | 7.0 |
| Thyroid | 0.0 | Renal ca. ACHN | 2.1 |
| Salivary gland | 0.0 | Renal ca. UO-31 | 0.0 |
| Pituitary gland | 0.0 | Renal ca. TK-10 | 0.0 |
| Brain (fetal) | 0.0 | Liver | 41.5 |
| Brain (whole) | 0.6 | Liver (fetal) | 76.8 |
| Brain (amygdala) | 4.0 | Liver ca. | 8.0 |
| | | (hepatoblast) HepG2 | |
| Brain (cerebellum) | 2.0 | Lung | 26.8 |
| Brain (hippocampus) | 0.0 | Lung (fetal) | 11.5 |
| Brain (substantia nigra) | 1.9 | Lung ca. (small cell) | 100.0 |
| | | LX-1 | |
| Brain (thalamus) | 4.1 | Lung ca. (small cell) | 0.0 |
| | | NCI-H69 | |
| Cerebral Cortex | 4.2 | Lung ca. (s.cell var.) | 0.0 |
| | | SHP-77 | |
| Spinal cord | 2.6 | Lung ca. (large | 19.1 |
| | | cell)NCI-H460 | |
| glio/astro U87-MG | 0.0 | Lung ca. (non-sm. | 16.6 |
| | | cell) A549 | |
| glio/astro U-118-MG | 0.0 | Lung ca. (non-s.cell) | 28.3 |
| | | NCI-H23 | |
| astrocytoma SW1783 | 2.1 | Lung ca. (non-s.cell) | 10.4 |
| | | HOP-62 | |
| neuro*; met SK-N-AS | 1.3 | Lung ca. (non-s.cl) | 0.0 |
| | | NCI-H522 | |
| astrocytoma SF-539 | 0.0 | Lung ca. (squam.) | 0.0 |
| | | SW 900 | |
| astrocytoma SNB-75 | 0.0 | Lung ca. (squam.) | 0.0 |
| | | NCI-H596 | |
| glioma SNB-19 | 0.0 | Mammary gland | 0.0 |
| glioma U251 | 0.0 | Breast ca.* (pl.ef) | 0.0 |
| | | MCF-7 | |
| glioma SF-295 | 0.0 | Breast ca.* (pl.ef) | 0.7 |
| | | MDA-MB-231 | |
| Heart (fetal) | 7.3 | Breast ca.* (pl.ef) | 0.0 |
| | | T47D | |
| Heart | 0.0 | Breast ca. BT-549 | 0.0 |
| Skeletal muscle (fetal) | 0.0 | Breast ca. MDA-N | 0.0 |
| Skeletal muscle | 0.0 | Ovary | 0.0 |
| Bone marrow | 77.4 | Ovarian ca. OVCAR-3 | 0.0 |
| Thymus | 0.0 | Ovarian ca. OVCAR-4 | 2.2 |
| Spleen | 0.0 | Ovarian ca. OVCAR-5 | 0.0 |
| Lymph node | 0.0 | Ovarian ca. OVCAR-8 | 0.0 |
| Colorectal | 0.0 | Ovarian ca. IGROV-1 | 0.0 |
| Stomach | 5.6 | Ovarian ca.* (ascites) | 0.0 |
| | | SK-OV-3 | |
| Small intestine | 0.0 | Uterus | 2.7 |
| Colon ca. SW480 | 32.3 | Placenta | 0.0 |
| Colon ca.* | 31.6 | Prostate | 0.0 |
| SW620(SW480 met) | | | |
| Colon ca. HT29 | 1.6 | Prostate ca.* (bone | 25.0 |
| | | met)PC-3 | |
| Colon ca. HCT-116 | 28.9 | Testis | 39.2 |
| Colon ca. CaCo-2 | 0.0 | Melanoma | 0.0 |
| | | Hs688(A).T | |
| Colon ca. | 1.4 | Melanoma* (met) | 0.0 |
| tissue(ODO3866) | | Hs688(B).T | |
| Colon ca. HCC-2998 | 0.0 | Melanoma UACC-62 | 14.2 |
| Gastric ca.* (liver met) | 3.4 | Melanoma M14 | 0.0 |
| NCI-N87 | | | |
| Bladder | 0.0 | Melanoma LOX | 0.0 |
| | | IMVI | |
| Trachea | 0.0 | Melanoma* (met) | 4.4 |
| | | SK-MEL-5 | |
| Kidney | 3.2 | Adipose | 4.1 |

TABLE AAC
Panel 2D

| | Rel. Exp. (%) | | Rel. Exp. (%) |
|---|---|---|---|
| | Ag2892, Run | | Ag2892, Run |
| Tissue Name | 160942674 | Tissue Name | 160942674 |
| Normal Colon | 1.8 | Kidney Margin | 0.9 |
| | | 8120608 | |
| CC Well to Mod Diff | 1.0 | Kidney Cancer | 0.0 |
| (ODO3866) | | 8120613 | |
| CC Margin (ODO3866) | 0.0 | Kidney Margin | 0.7 |
| | | 8120614 | |
| CC Gr.2 rectosigmoid | 0.0 | Kidney Cancer | 2.0 |
| (ODO3868) | | 9010320 | |
| CC Margin (ODO3868) | 0.0 | Kidney Margin | 0.0 |
| | | 9010321 | |
| CC Mod Diff (ODO3920) | 2.4 | Normal Uterus | 0.0 |
| CC Margin (ODO3920) | 0.0 | Uterus Cancer 064011 | 1.1 |
| CC Gr.2 ascend colon | 1.0 | Normal Thyroid | 0.0 |
| (ODO3921) | | | |
| CC Margin (ODO3921) | 0.0 | Thyroid Cancer | 1.5 |
| | | 064010 | |
| CC from Partial | 5.3 | Thyroid Cancer | 1.6 |
| Hepatectomy (ODO4309) | | A302152 | |
| Mets | | | |
| Liver Margin (ODO4309) | 100.0 | Thyroid Margin | 0.0 |
| | | A302153 | |
| Colon mets to lung | 5.6 | Normal Breast | 0.7 |
| (OD04451-01) | | | |
| Lung Margin (OD04451- | 4.2 | Breast Cancer | 0.0 |
| 02) | | (OD04566) | |
| Normal Prostate 6546-1 | 0.0 | Breast Cancer | 0.0 |
| | | (OD04590-01) | |
| Prostate Cancer | 0.0 | Breast Cancer Mets | 0.0 |
| (OD04410) | | (OD04590-03) | |
| Prostate Margin | 0.7 | Breast Cancer | 3.3 |
| (OD04410) | | Metastasis | |
| | | (OD04655-05) | |
| Prostate Cancer | 0.0 | Breast Cancer 064006 | 0.0 |
| (OD04720-01) | | | |
| Prostate Margin | 0.7 | Breast Cancer 1024 | 0.0 |
| (OD04720-02) | | | |
| Normal Lung 061010 | 6.7 | Breast Cancer | 0.0 |
| | | 9100266 | |
| Lung Met to Muscle | 3.4 | Breast Margin | 0.0 |
| (ODO4286) | | 9100265 | |
| Muscle Margin | 0.0 | Breast Cancer | 0.0 |
| (ODO4286) | | A209073 | |
| Lung Malignant Cancer | 1.0 | Breast Margin | 0.0 |
| (OD03126) | | A2090734 | |
| Lung Margin (OD03126) | 9.2 | Normal Liver | 59.0 |
| Lung Cancer (OD04404) | 3.5 | Liver Cancer 064003 | 19.2 |
| Lung Margin (OD04404) | 8.2 | Liver Cancer 1025 | 60.7 |
| Lung Cancer (OD04565) | 1.4 | Liver Cancer 1026 | 39.5 |
| Lung Margin (OD04565) | 12.3 | Liver Cancer 6004-T | 73.2 |
| Lung Cancer (OD04237- | 0.0 | Liver Tissue 6004-N | 9.8 |
| 01) | | | |
| Lung Margin (OD04237- | 7.2 | Liver Cancer 6005-T | 39.5 |
| 02) | | | |
| Ocular Mel Met to Liver | 0.6 | Liver Tissue 6005-N | 7.9 |
| (ODO4310) | | | |
| Liver Margin (ODO4310) | 23.0 | Normal Bladder | 0.0 |
| Melanoma Mets to Lung | 1.0 | Bladder Cancer 1023 | 0.0 |
| (OD04321) | | | |
| Lung Margin (OD04321) | 18.8 | Bladder Cancer | 0.0 |
| | | A302173 | |
| Normal Kidney | 2.0 | Bladder Cancer | 6.1 |
| | | (OD04718-01) | |
| Kidney Ca, Nuclear grade | 1.2 | Bladder Normal | 0.0 |
| 2 (OD04338) | | Adjacent (OD04718- | |
| | | 03) | |
| Kidney Margin | 1.0 | Normal Ovary | 0.0 |
| (OD04338) | | | |
| Kidney Ca Nuclear grade | 0.0 | Ovarian cancer | 1.8 |
| 1/2 (OD04339) | | 064008 | |
| Kidney Margin | 0.0 | Ovarian Cancer | 0.0 |
| (OD04339) | | (OD04768-07) | |
| Kidney Ca, Clear cell | 0.0 | Ovary Margin | 0.6 |
| type (OD04340) | | (OD04768-08) | |
| Kidney Margin | 3.3 | Normal Stomach | 1.6 |
| (OD04340) | | | |
| Kidney Ca, Nuclear grade | 0.0 | Gastric Cancer | 0.6 |
| 3 (OD04348) | | 9060358 | |
| Kidney Margin | 0.0 | Stomach Margin | 1.7 |
| (OD04348) | | 9060359 | |
| Kidney Cancer | 0.0 | Gastric Cancer | 0.0 |
| (OD04622-01) | | 9060395 | |
| Kidney Margin | 0.8 | Stomach Margin | 0.6 |
| (OD04622-03) | | 9060394 | |
| Kidney Cancer | 0.0 | Gastric Cancer | 0.7 |
| (OD04450-01) | | 9060397 | |
| Kidney Margin | 2.7 | Stomach Margin 9060396 | 0.9 |
| (OD04450-03) | | | |
| Kidney Cancer 8120607 | 1.4 | Gastric Cancer | 0.0 |
| | | 064005 | |

TABLE AAD
Panel 3D

| | Rel. Exp. (%) | | Rel. Exp. (%) |
|---|---|---|---|
| | Ag2892, Run | | Ag2892, Run |
| Tissue Name | 164629840 | Tissue Name | 164629840 |
| Daoy-Medulloblastoma | 5.0 | Ca Ski-Cervical epidermoid | 0.0 |
| | | carcinoma (metastasis) | |
| TE671-Medulloblastoma | 0.0 | ES-2-Ovarian clear cell | 0.0 |
| | | carcinoma | |
| D283 Med- | 0.0 | Ramos-Stimulated with | 0.0 |
| Medulloblastoma | | PMA/ionomycin 6 h | |
| PFSK-1-Primitive | 0.0 | Ramos-Stimulated with | 0.0 |
| Neuroectodermal | | PMA/ionomycin 14 h | |
| XF-498-CNS | 0.0 | MEG-01-Chronic | 0.0 |
| | | myelogenous leukemia | |
| | | (megokaryoblast) | |
| SNB-78-Glioma | 0.0 | Raji-Burkitt's lymphoma | 0.0 |
| SF-268-Glioblastoma | 0.0 | Daudi-Burkitt's lymphoma | 0.0 |
| T98G-Glioblastoma | 0.0 | U266-B-cell plasmacytoma | 0.0 |
| SK-N-SH- | 0.0 | CA46-Burkitt's lymphoma | 0.0 |
| Neuroblastoma | | | |
| (metastasis) | | | |
| SF-295-Glioblastoma | 0.0 | RL-non-Hodgkin's B-cell | 0.0 |
| | | lymphoma | |
| Cerebellum | 2.2 | JM1-pre-B-cell lymphoma | 0.0 |
| Cerebellum | 3.2 | Jurkat-T cell leukemia | 0.0 |
| NCI-H292- | 0.0 | TF-1-Erythroleukemia | 0.0 |
| Mucoepidermoid lung | | | |
| carcinoma | | | |
| DMS-114-Small cell | 22.8 | HUT 78-T-cell lymphoma | 0.0 |
| lung cancer | | | |
| DMS-79-Small cell lung | 0.0 | U937-Histiocytic lymphoma | 0.0 |
| cancer | | | |
| NCI-H146-Small cell | 0.0 | KU-812-Myelogenous | 14.5 |
| lung cancer | | leukemia | |
| NCI-H526-Small cell | 0.0 | 769-P-Clear cell renal | 0.0 |
| lung cancer | | carcinoma | |
| NCI-N417-Small cell | 0.0 | Caki-2-Clear cell renal | 2.0 |
| lung cancer | | carcinoma | |
| NCI-H82-Small cell | 0.0 | SW 839-Clear cell renal | 1.7 |
| lung cancer | | carcinoma | |
| NCI-H157-Squamous | 20.4 | G401-Wilms' tumor | 0.5 |
| cell lung cancer | | | |
| (metastasis) | | | |
| NCI-H1155-Large cell | 0.0 | Hs766T-Pancreatic | 1.0 |
| lung cancer | | carcinoma (LN metastasis) | |
| NCI-H1299-Large cell | 0.0 | CAPAN-1-Pancreatic | 2.0 |
| lung cancer | | adenocarcinoma (liver | |
| | | metastasis) | |
| NCI-H727-Lung | 0.0 | SU86.86-Pancreatic | 0.5 |
| carcinoid | | carcinoma (liver metastasis) | |
| NCI-UMC-11-Lung | 0.0 | BxPC-3-Pancreatic | 0.0 |
| carcinoid | | adenocarcinoma | |
| LX-1-Small cell lung | 100.0 | HPAC-Pancreatic | 0.0 |
| cancer | | adenocarcinoma | |
| Colo-205-Colon cancer | 0.0 | MIA PaCa-2-Pancreatic | 2.0 |
| | | carcinoma | |
| KM12-Colon cancer | 0.0 | CFPAC-1-Pancreatic ductal | 0.7 |
| | | adenocarcinoma | |
| KM20L2-Colon cancer | 0.6 | PANC-1-Pancreatic | 6.5 |
| | | epithelioid ductal carcinoma | |
| NCI-H716-Colon cancer | 0.4 | T24-Bladder carcinma | 0.0 |
| | | (transitional cell) | |
| SW-48-Colon | 6.6 | 5637-Bladder carcinoma | 0.0 |
| adenocarcinoma | | | |
| SW1116-Colon | 0.0 | HT-1197-Bladder carcinoma | 0.0 |
| adenocarcinoma | | | |
| LS 174T-Colon | 0.0 | UM-UC-3-Bladder carcinma | 4.4 |
| adenocarcinoma | | (transitional cell) | |
| SW-948-Colon | 0.0 | A204-Rhabdomyosarcoma | 0.0 |
| adenocarcinoma | | | |
| SW-480-Colon | 2.8 | HT-1080-Fibrosarcoma | 0.0 |
| adenocarcinoma | | | |
| NCI-SNU-5-Gastric | 63.7 | MG-63-Osteosarcoma | 4.3 |
| carcinoma | | | |
| KATO III-Gastric | 23.7 | SK-LMS-1-Leiomyosarcoma | 0.0 |
| carcinoma | | (vulva) | |
| NCI-SNU-16-Gastric | 1.3 | SJRH30-Rhabdomyosarcoma | 0.0 |
| carcinoma | | (met to bone marrow) | |
| NCI-SNU-1-Gastric | 0.0 | A431-Epidermoid carcinoma | 6.9 |
| carcinoma | | | |
| RF-1-Gastric | 0.0 | WM266-4-Melanoma | 13.1 |
| adenocarcinoma | | | |
| RF-48-Gastric | 0.0 | DU 145-Prostate carcinoma | 0.0 |
| adenocarcinoma | | (brain metastasis) | |
| MKN-45-Gastric | 0.0 | MDA-MB-468-Breast | 5.0 |
| carcinoma | | adenocarcinoma | |
| NCI-N87-Gastric | 2.1 | SCC-4-Squamous cell | 0.0 |
| carcinoma | | carcinoma of tongue | |
| OVCAR-5-Ovarian | 0.0 | SCC-9-Squamous cell | 0.0 |
| carcinoma | | carcinoma of tongue | |
| RL95-2-Uterine | 0.0 | SCC-15-Squamous cell | 0.0 |
| carcinoma | | carcinoma of tongue | |
| HelaS3-Cervical | 8.8 | CAL 27-Squamous cell | 0.0 |
| adenocarcinoma | | carcinoma of tongue | |

TABLE AAE
Panel 4D

| | Rel. Exp. (%) | | Rel. Exp. (%) |
|---|---|---|---|
| | Ag2892, Run | | Ag2892, Run |
| Tissue Name | 164033139 | Tissue Name | 164033139 |
| Secondary Th1 act | 0.0 | HUVEC IL-1beta | 0.0 |
| Secondary Th2 act | 0.0 | HUVEC IFN gamma | 0.0 |
| Secondary Tr1 act | 0.0 | HUVEC TNF alpha + IFN | 0.0 |
| | | gamma | |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC | 0.0 |
| | | none | |
| Primary Th1 act | 0.0 | Lung Microvascular EC | 0.0 |
| | | TNF alpha + IL-1beta | |
| Primary Th2 act | 0.0 | Microvascular Dermal EC | 0.0 |
| | | none | |
| Primary Tr1 act | 0.0 | Microsvasular Dermal EC | 0.0 |
| | | TNF alpha + IL-1beta | |
| Primary Th1 rest | 0.0 | Bronchial epithelium | 0.0 |
| | | TNF alpha + IL-1beta | |
| Primary Th2 rest | 0.0 | Small airway epithelium | 21.5 |
| | | none | |
| Primary Tr1 rest | 0.0 | Small airway epithelium | 65.1 |
| | | TNF alpha + IL-1beta | |
| CD45RA CD4 | 6.7 | Coronery artery SMC rest | 0.0 |
| lymphocyte act | | | |
| CD45RO CD4 | 0.0 | Coronery artery SMC | 0.0 |
| lymphocyte act | | TNF alpha + IL-1beta | |
| CD8 lymphocyte act | 0.0 | Astrocytes rest | 0.0 |
| Secondary CD8 | 0.0 | Astrocytes TNF alpha + | 0.0 |
| lymphocyte rest | | IL-1beta | |
| Secondary CD8 | 0.0 | KU-812 (Basophil) rest | 32.5 |
| lymphocyte act | | | |
| CD4 lymphocyte none | 0.0 | KU-812 (Basophil) | 31.6 |
| | | PMA/ionomycin | |
| 2ry Th1/Th2/Tr1_anti- | 0.0 | CCD1106 (Keratinocytes) | 0.0 |
| CD95 CH11 | | none | |
| LAK cells rest | 0.0 | CCD1106 (Keratinocytes) | 3.8 |
| | | TNF alpha + IL-1beta | |
| LAK cells IL-2 | 0.0 | Liver cirrhosis | 100.0 |
| LAK cells IL-2 + IL-12 | 0.0 | Lupus kidney | 6.8 |
| LAK cells IL-2 + IFN | 0.0 | NCI-H292 none | 11.0 |
| gamma | | | |
| LAK cells IL-2 + IL-18 | 0.0 | NCI-H292 IL-4 | 0.0 |
| LAK cells | 0.0 | NCI-H292 IL-9 | 0.0 |
| PMA/ionomycin | | | |
| NK Cells IL-2 rest | 0.0 | NCI-H292 IL-13 | 0.0 |
| Two Way MLR 3 day | 0.0 | NCI-H292 IFN gamma | 0.0 |
| Two Way MLR 5 day | 0.0 | HPAEC none | 0.0 |
| Two Way MLR 7 day | 0.0 | HPAEC TNF alpha + IL- | 0.0 |
| | | 1beta | |
| PBMC rest | 0.0 | Lung fibroblast none | 0.0 |
| PBMC PWM | 0.0 | Lung fibroblast TNF alpha + | 8.1 |
| | | IL-1beta | |
| PBMC PHA-L | 0.0 | Lung fibroblast IL-4 | 10.4 |
| Ramos (B cell) none | 0.0 | Lung fibroblast IL-9 | 0.0 |
| Ramos (B cell) | 0.0 | Lung fibroblast IL-13 | 4.3 |
| ionomycin | | | |
| B lymphocytes PWM | 4.5 | Lung fibroblast IFN | 5.8 |
| | | gamma | |
| B lymphocytes CD40L | 0.0 | Dermal fibroblast | 0.0 |
| and IL-4 | | CCD1070 rest | |
| EOL-1 dbcAMP | 0.0 | Dermal fibroblast | 10.4 |
| | | CCD1070 TNF alpha | |
| EOL-1 dbcAMP | 0.0 | Dermal fibroblast | 0.0 |
| PMA/ionomycin | | CCD1070 IL-1beta | |
| Dendritic cells none | 0.0 | Dermal fibroblast IFN | 13.0 |
| | | gamma | |
| Dendritic cells LPS | 0.0 | Dermal fibroblast IL-4 | 21.0 |
| Dendritic cells anti- | 0.0 | IBD Colitis 2 | 0.0 |
| CD40 | | | |
| Monocytes rest | 0.0 | IBD Crohn's | 0.0 |
| Monocytes LPS | 0.0 | Colon | 12.2 |
| Macrophages rest | 0.0 | Lung | 63.7 |
| Macrophages LPS | 0.0 | Thymus | 43.8 |
| HUVEC none | 0.0 | Kidney | 0.0 |
| HUVEC starved | 0.0 | | |

Panel 1.3D Summary: Ag2892 Highest expression of the NOV29a gene is seen in a lung cancer cell line (CT=32.7). Significant expression is also seen in a colon cancer cell line and the liver. Thus, expression of this gene could be used to differentiate between these samples and other samples on this panel and as a diagnostic marker for the presence of colon and lung cancer. Furthermore, therapeutic modulation of the expression or function of this gene may be effective in the treatment of colon and lung cancers. Results from a second run with the same probe and primer set are not included because of a potential problem in one of the sample wells.

Panel 2D Summary: Ag2892 Expression of the NOV29a gene is restricted to liver derived tissue, with highest expression in normal liver tissue (CT=32.4). Significant expression is also seen in liver cancer samples. Thus, expression of this gene could be used to differentiate liver derived samples from other samples on this panel and from other tissue samples.

Panel 3D Summary: Ag2892 Highest expression of the NOV29a gene is seen in a lung cancer cell line (CT=31.4). Significant expression is also seen in a gastric cancer cell line. Thus, expression of this gene could be used to differentiate between these samples and other samples on this panel and as a diagnostic marker for the presence of gastric and lung cancer. Furthermore, therapeutic modulation of the expression or function of this gene may be effective in the treatment of gastric and lung cancers.

Panel 4D Summary: Ag2892 Expression of the NOV29a gene is restricted to liver cirrhosis (CT=34.8). This liver specific expression is in agreement with the expression in Panels 1.3D and 2D. Thus, expression of this gene could be used to differentiate between this sample and other samples on this panel, and as a marker of liver tissue.

NOV29c

Expression of gene NOV29c was assessed using the primer-probe set Ag2893, described in Table ABA. Results of the RTQ-PCR runs are shown in Tables ABB, ABC, ABD, ABE and ABF.

TABLE ABA
Probe Name Ag2893

| | | | Start | |
|---|---|---|---|---|
| Primers | Sequence | Length | Position | SEQ ID NO: |
| Forward | 5′-gcccaatcctgatgactacttc-3′ | 22 | 39 | 1093 |
| Probe | TET-5′-ctccaagctcggagctttgacctg-3′-TAMRA | 24 | 73 | 1094 |
| Reverse | 5′-ctcagcatgtcctctgatttct-3′ | 22 | 98 | 1095 |

TABLE ABB
CNS_neurodegeneration_v1.0

| | Rel. Exp. (%) Ag2893, | | Rel. Exp. (%) Ag2893, |
|---|---|---|---|
| Tissue Name | Run 224116295 | Tissue Name | Run 224116295 |
| AD 1 Hippo | 18.6 | Control (Path) 3 | 15.8 |
| | | Temporal Ctx | |
| AD 2 Hippo | 59.0 | Control (Path) 4 | 43.5 |
| | | Temporal Ctx | |
| AD 3 Hippo | 25.3 | AD 1 Occipital Ctx | 41.8 |
| AD 4 Hippo | 56.3 | AD 2 Occipital Ctx | 0.0 |
| | | (Missing) | |
| AD 5 Hippo | 95.9 | AD 3 Occipital Ctx | 27.5 |
| AD 6 Hippo | 86.5 | AD 4 Occipital Ctx | 58.6 |
| Control 2 Hippo | 26.1 | AD 5 Occipital Ctx | 47.0 |
| Control 4 Hippo | 59.5 | AD 6 Occipital Ctx | 31.2 |
| Control (Path) 3 | 17.3 | Control 1 Occipital | 20.9 |
| Hippo | | Ctx | |
| AD 1 Temporal Ctx | 48.6 | Control 2 Occipital | 63.7 |
| | | Ctx | |
| AD 2 Temporal Ctx | 61.1 | Control 3 Occipital | 62.0 |
| | | Ctx | |
| AD 3 Temporal Ctx | 25.2 | Control 4 Occipital | 28.1 |
| | | Ctx | |
| AD 4 Temporal Ctx | 70.7 | Control (Path) 1 | 82.9 |
| | | Occipital Ctx | |
| AD 5 Inf Temporal | 100.0 | Control (Path) 2 | 24.5 |
| Ctx | | Occipital Ctx | |
| AD 5 Sup Temporal | 55.9 | Control (Path) 3 | 15.0 |
| Ctx | | Occipital Ctx | |
| AD 6 Inf Temporal | 86.5 | Control (Path) 4 | 28.3 |
| Ctx | | Occipital Ctx | |
| AD 6 Sup Temporal | 92.7 | Control 1 Parietal | 20.4 |
| Ctx | | Ctx | |
| Control 1 Temporal | 24.7 | Control 2 Parietal | 55.1 |
| Ctx | | Ctx | |
| Control 2 Temporal | 53.2 | Control 3 Parietal | 30.4 |
| Ctx | | Ctx | |
| Control 3 Temporal | 26.8 | Control (Path) 1 | 74.2 |
| Ctx | | Parietal Ctx | |
| Control 3 Temporal | 40.6 | Control (Path) 2 | 35.8 |
| Ctx | | Parietal Ctx | |
| Control (Path) 1 | 63.7 | Control (Path) 3 | 6.9 |
| Temporal Ctx | | Parietal Ctx | |
| Control (Path) 2 | 44.1 | Control (Path) 4 | 38.4 |
| Temporal Ctx | | Parietal Ctx | |

TABLE ABC
Panel 1.3D

| | Rel. Exp. (%) | Rel. Exp. (%) | | Rel. Exp. (%) | Rel. Exp. (%) |
|---|---|---|---|---|---|
| | Ag2893, Run | Ag2893, Run | | Ag2893, Run | Ag2893, Run |
| Tissue Name | 160944329 | 165701489 | Tissue Name | 160944329 | 165701489 |
| Liver | 0.0 | 0.0 | Kidney (fetal) | 19.5 | 4.3 |
| adenocarcinoma | | | | | |
| Pancreas | 2.5 | 4.0 | Renal ca. 786-0 | 73.7 | 64.2 |
| Pancreatic ca. | 0.0 | 0.0 | Renal ca. | 0.5 | 0.6 |
| CAPAN 2 | | | A498 | | |
| Adrenal gland | 0.8 | 1.2 | Renal ca. RXF | 100.0 | 100.0 |
| | | | 393 | | |
| Thyroid | 0.5 | 0.3 | Renal ca. | 28.1 | 18.8 |
| | | | ACHN | | |
| Salivary gland | 0.0 | 0.0 | Renal ca. UO- | 23.5 | 24.3 |
| | | | 31 | | |
| Pituitary gland | 2.0 | 1.8 | Renal ca. TK- | 21.2 | 8.9 |
| | | | 10 | | |
| Brain (fetal) | 0.9 | 2.0 | Liver | 0.0 | 1.3 |
| Brain (whole) | 3.6 | 6.3 | Liver (fetal) | 1.2 | 1.6 |
| Brain (amygdala) | 3.6 | 6.1 | Liver ca. | 10.9 | 8.1 |
| | | | (hepatoblast) | | |
| | | | HepG2 | | |
| Brain (cerebellum) | 6.3 | 6.2 | Lung | 8.9 | 4.8 |
| Brain | 9.3 | 10.4 | Lung (fetal) | 6.6 | 3.6 |
| (hippocampus) | | | | | |
| Brain (substantia | 2.6 | 5.4 | Lung ca. | 0.0 | 0.0 |
| nigra) | | | (small cell) | | |
| | | | LX-1 | | |
| Brain (thalamus) | 7.2 | 13.8 | Lung ca. | 0.0 | 0.0 |
| | | | (small cell) | | |
| | | | NCI-H69 | | |
| Cerebral Cortex | 25.5 | 3.3 | Lung ca. | 0.0 | 0.0 |
| | | | (s.cell var.) | | |
| | | | SHP-77 | | |
| Spinal cord | 17.0 | 10.7 | Lung ca. (large | 0.0 | 0.0 |
| | | | cell)NCI-H460 | | |
| glio/astro U87-MG | 0.0 | 0.0 | Lung ca. (non- | 0.0 | 0.0 |
| | | | sm. cell) A549 | | |
| glio/astro U-118- | 0.0 | 0.0 | Lung ca. (non- | 2.3 | 1.1 |
| MG | | | s.cell) NCI- | | |
| | | | H23 | | |
| astrocytoma | 0.6 | 0.0 | Lung ca. (non- | 3.6 | 2.5 |
| SW1783 | | | s.cell) HOP-62 | | |
| neuro*; met SK-N- | 0.0 | 0.0 | Lung ca. (non- | 0.0 | 0.0 |
| AS | | | s.cl) NCI- | | |
| | | | H522 | | |
| astrocytoma SF- | 0.0 | 0.0 | Lung ca. | 0.0 | 2.4 |
| 539 | | | (squam.) SW | | |
| | | | 900 | | |
| astrocytoma SNB- | 0.0 | 0.0 | Lung ca. | 0.8 | 0.0 |
| 75 | | | (squam.) NCI- | | |
| | | | H596 | | |
| glioma SNB-19 | 0.0 | 0.0 | Mammary | 0.5 | 0.0 |
| | | | gland | | |
| glioma U251 | 0.0 | 0.0 | Breast ca.* | 0.0 | 0.0 |
| | | | (pl.ef) MCF-7 | | |
| glioma SF-295 | 0.0 | 0.0 | Breast ca.* | 0.4 | 1.7 |
| | | | (pl.ef) MDA- | | |
| | | | MB-231 | | |
| Heart (fetal) | 1.4 | 0.0 | Breast ca.* | 4.8 | 0.9 |
| | | | (pl.ef) T47D | | |
| Heart | 0.0 | 0.0 | Breast ca. BT- | 0.0 | 0.0 |
| | | | 549 | | |
| Skeletal muscle | 3.3 | 0.0 | Breast ca. | 0.0 | 0.0 |
| (fetal) | | | MDA-N | | |
| Skeletal muscle | 0.4 | 0.0 | Ovary | 9.9 | 2.4 |
| Bone marrow | 0.0 | 0.3 | Ovarian ca. | 3.2 | 2.2 |
| | | | OVCAR-3 | | |
| Thymus | 1.4 | 0.2 | Ovarian ca. | 0.0 | 0.0 |
| | | | OVCAR-4 | | |
| Spleen | 1.7 | 0.6 | Ovarian ca. | 0.0 | 0.0 |
| | | | OVCAR-5 | | |
| Lymph node | 0.0 | 1.1 | Ovarian ca. | 1.0 | 1.2 |
| | | | OVCAR-8 | | |
| Colorectal | 0.0 | 0.0 | Ovarian ca. | 0.0 | 0.0 |
| | | | IGROV-1 | | |
| Stomach | 0.0 | 2.0 | Ovarian ca.* | 0.0 | 0.0 |
| | | | (ascites) SK- | | |
| | | | OV-3 | | |
| Small intestine | 0.0 | 2.3 | Uterus | 1.9 | 5.4 |
| Colon ca. SW480 | 2.3 | 0.5 | Placenta | 0.5 | 0.9 |
| Colon ca.* | 0.0 | 0.6 | Prostate | 1.0 | 1.0 |
| SW620(SW480 | | | | | |
| met) | | | | | |
| Colon ca. HT29 | 0.0 | 0.0 | Prostate ca.* | 0.0 | 0.0 |
| | | | (bone met)PC-3 | | |
| Colon ca. HCT- | 0.0 | 0.0 | Testis | 25.7 | 9.3 |
| 116 | | | | | |
| Colon ca. CaCo-2 | 0.9 | 0.0 | Melanoma | 0.0 | 0.0 |
| | | | Hs688(A).T | | |
| Colon ca. | 1.5 | 0.0 | Melanoma* | 0.0 | 0.0 |
| tissue(ODO3866) | | | (met) | | |
| | | | Hs688(B).T | | |
| Colon ca. HCC- | 0.0 | 0.0 | Melanoma | 0.0 | 0.0 |
| 2998 | | | UACC-62 | | |
| Gastric ca.* (liver | 0.0 | 0.6 | Melanoma | 0.0 | 0.0 |
| met) NCI-N87 | | | M14 | | |
| Bladder | 11.3 | 5.0 | Melanoma | 0.0 | 0.0 |
| | | | LOX IMVI | | |
| Trachea | 2.5 | 0.0 | Melanoma* | 0.0 | 0.0 |
| | | | (met) SK- | | |
| | | | MEL-5 | | |
| Kidney | 26.2 | 5.3 | Adipose | 1.0 | 0.0 |

TABLE ABD
Panel 2D

| | Rel. Exp. (%) | | Rel. Exp. (%) |
|---|---|---|---|
| | Ag2893, Run | | Ag2893, Run |
| Tissue Name | 160966072 | Tissue Name | 160966072 |
| Normal Colon | 2.5 | Kidney Margin | 16.8 |
| | | 8120608 | |
| CC Well to Mod Diff | 0.0 | Kidney Cancer | 0.0 |
| (ODO3866) | | 8120613 | |
| CC Margin (ODO3866) | 0.5 | Kidney Margin | 18.0 |
| | | 8120614 | |
| CC Gr.2 rectosigmoid | 0.0 | Kidney Cancer | 74.7 |
| (ODO3868) | | 9010320 | |
| CC Margin (ODO3868) | 0.0 | Kidney Margin | 18.3 |
| | | 9010321 | |
| CC Mod Diff (ODO3920) | 0.5 | Normal Uterus | 0.7 |
| CC Margin (ODO3920) | 0.4 | Uterus Cancer 064011 | 10.8 |
| CC Gr.2 ascend colon | 0.5 | Normal Thyroid | 0.0 |
| (ODO3921) | | | |
| CC Margin (ODO3921) | 0.0 | Thyroid Cancer | 1.7 |
| | | 064010 | |
| CC from Partial | 0.7 | Thyroid Cancer | 3.4 |
| Hepatectomy (ODO4309) | | A302152 | |
| Mets | | | |
| Liver Margin (ODO4309) | 0.9 | Thyroid Margin | 0.0 |
| | | A302153 | |
| Colon mets to lung | 5.0 | Normal Breast | 2.0 |
| (OD04451-01) | | | |
| Lung Margin (OD04451- | 18.0 | Breast Cancer | 0.2 |
| 02) | | (OD04566) | |
| Normal Prostate 6546-1 | 0.7 | Breast Cancer | 0.2 |
| | | (OD04590-01) | |
| Prostate Cancer | 2.0 | Breast Cancer Mets | 0.8 |
| (OD04410) | | (OD04590-03) | |
| Prostate Margin | 8.7 | Breast Cancer | 0.6 |
| (OD04410) | | Metastasis | |
| | | (OD04655-05) | |
| Prostate Cancer | 3.8 | Breast Cancer 064006 | 0.3 |
| (OD04720-01) | | | |
| Prostate Margin | 3.5 | Breast Cancer 1024 | 0.5 |
| (OD04720-02) | | | |
| Normal Lung 061010 | 12.9 | Breast Cancer | 0.5 |
| | | 9100266 | |
| Lung Met to Muscle | 0.0 | Breast Margin | 1.1 |
| (ODO4286) | | 9100265 | |
| Muscle Margin | 0.3 | Breast Cancer | 0.2 |
| (ODO4286) | | A209073 | |
| Lung Malignant Cancer | 8.7 | Breast Margin | 0.4 |
| (OD03126) | | A2090734 | |
| Lung Margin (OD03126) | 21.0 | Normal Liver | 0.0 |
| Lung Cancer (OD04404) | 8.0 | Liver Cancer 064003 | 0.8 |
| Lung Margin (OD04404) | 28.7 | Liver Cancer 1025 | 0.4 |
| Lung Cancer (OD04565) | 0.7 | Liver Cancer 1026 | 8.1 |
| Lung Margin (OD04565) | 33.0 | Liver Cancer 6004-T | 1.2 |
| Lung Cancer (OD04237- | 1.3 | Liver Tissue 6004-N | 0.0 |
| 01) | | | |
| Lung Margin (OD04237- | 11.3 | Liver Cancer 6005-T | 5.4 |
| 02) | | | |
| Ocular Mel Met to Liver | 0.0 | Liver Tissue 6005-N | 0.3 |
| (ODO4310) | | | |
| Liver Margin (ODO4310) | 0.4 | Normal Bladder | 17.3 |
| Melanoma Mets to Lung | 1.0 | Bladder Cancer 1023 | 0.2 |
| (OD04321) | | | |
| Lung Margin (OD04321) | 32.1 | Bladder Cancer | 1.3 |
| | | A302173 | |
| Normal Kidney | 42.0 | Bladder Cancer | 7.9 |
| | | (OD04718-01) | |
| Kidney Ca, Nuclear grade | 55.5 | Bladder Normal | 0.4 |
| 2 (OD04338) | | Adjacent (OD04718- | |
| | | 03) | |
| Kidney Margin | 40.9 | Normal Ovary | 2.0 |
| (OD04338) | | | |
| Kidney Ca Nuclear grade | 61.1 | Ovarian Cancer | 10.7 |
| 1/2 (OD04339) | | 064008 | |
| Kidney Margin | 22.8 | Ovarian Cancer | 0.3 |
| (OD04339) | | (OD04768-07) | |
| Kidney Ca, Clear cell | 100.0 | Ovary Margin | 4.2 |
| type (OD04340) | | (OD04768-08) | |
| Kidney Margin | 31.9 | Normal Stomach | 3.1 |
| (OD04340) | | | |
| Kidney Ca, Nuclear grade | 3.3 | Gastric Cancer | 0.4 |
| 3 (OD04348) | | 9060358 | |
| Kidney Margin | 27.0 | Stomach Margin | 1.1 |
| (OD04348) | | 9060359 | |
| Kidney Cancer | 30.4 | Gastric Cancer | 0.2 |
| (OD04622-01) | | 9060395 | |
| Kidney Margin | 17.4 | Stomach Margin | 3.0 |
| (OD04622-03) | | 9060394 | |
| Kidney Cancer | 21.5 | Gastric Cancer | 0.2 |
| (OD04450-01) | | 9060397 | |
| Kidney Margin | 21.3 | Stomach Margin 9060396 | 2.6 |
| (OD04450-03) | | | |
| Kidney Cancer 8120607 | 70.7 | Gastric Cancer | 0.8 |
| | | 064005 | |

TABLE ABE
Panel 3D

| | Rel. Exp. (%) | | Rel. Exp. (%) |
|---|---|---|---|
| | Ag2893, Run | | Ag2893, Run |
| Tissue Name | 165924139 | Tissue Name | 165924139 |
| Daoy-Medulloblastoma | 2.0 | Ca Ski-Cervical epidermoid | 0.0 |
| | | carcinoma (metastasis) | |
| TE671-Medulloblastoma | 0.0 | ES-2-Ovarian clear cell | 0.0 |
| | | carcinoma | |
| D283 Med- | 0.0 | Ramos-Stimulated with | 0.0 |
| Medulloblastoma | | PMA/ionomycin 6 h | |
| PFSK-1-Primitive | 0.0 | Ramos-Stimulated with | 0.0 |
| Neuroectodermal | | PMA/ionomycin 14 h | |
| XF-498-CNS | 0.0 | MEG-01-Chronic | 0.0 |
| | | myelogenous leukemia | |
| | | (megokaryoblast) | |
| SNB-78-Glioma | 0.0 | Raji-Burkitt's lymphoma | 0.0 |
| SF-268-Glioblastoma | 0.9 | Daudi-Burkitt's lymphoma | 0.0 |
| T98G-Glioblastoma | 0.0 | U266-B-cell plasmacytoma | 0.0 |
| SK-N-SH- | 0.0 | CA46-Burkitt's lymphoma | 0.0 |
| Neuroblastoma | | | |
| (metastasis) | | | |
| SF-295-Glioblastoma | 0.0 | RL-non-Hodgkin's B-cell | 0.0 |
| | | lymphoma | |
| Cerebellum | 6.4 | JM1-pre-B-cell lymphoma | 0.0 |
| Cerebellum | 9.9 | Jurkat-T cell leukemia | 0.0 |
| NCI-H292- | 2.0 | TF-1-Erythroleukemia | 0.0 |
| Mucoepidermoid lung | | | |
| carcinoma | | | |
| DMS-114-Small cell | 0.0 | HUT 78-T-cell lymphoma | 0.0 |
| lung cancer | | | |
| DMS-79-Small cell lung | 1.4 | U937-Histiocytic lymphoma | 0.0 |
| cancer | | | |
| NCI-H146-Small cell | 0.0 | KU-812-Myelogenous | 0.0 |
| lung cancer | | leukemia | |
| NCI-H526-Small cell | 4.5 | 769-P-Clear cell renal | 7.7 |
| lung cancer | | carcinoma | |
| NCI-N417-Small cell | 0.0 | Caki-2-Clear cell renal | 100.0 |
| lung cancer | | carcinoma | |
| NCI-H82-Small cell | 0.0 | SW 839-Clear cell renal | 73.2 |
| lung cancer | | carcinoma | |
| NCI-H157-Squamous | 0.0 | G401-Wilms' tumor | 0.0 |
| cell lung cancer | | | |
| (metastasis) | | | |
| NCI-H1155-Large cell | 0.0 | Hs766T-Pancreatic | 0.0 |
| lung cancer | | carcinoma (LN metastasis) | |
| NCI-H1299-Large cell | 0.0 | CAPAN-1-Pancreatic | 5.3 |
| lung cancer | | adenocarcinoma (liver | |
| | | metastasis) | |
| NCI-H727-Lung | 0.0 | SU86.86-Pancreatic | 1.3 |
| carcinoid | | carcinoma (liver metastasis) | |
| NCI-UMC-11-Lung | 0.0 | BxPC-3-Pancreatic | 0.0 |
| carcinoid | | adenocarcinoma | |
| LX-1-Small cell lung | 0.0 | HPAC-Pancreatic | 0.0 |
| cancer | | adenocarcinoma | |
| Colo-205-Colon cancer | 0.0 | MIA PaCa-2-Pancreatic | 0.0 |
| | | carcinoma | |
| KM12-Colon cancer | 0.0 | CFPAC-1-Pancreatic ductal | 1.6 |
| | | adenocarcinoma | |
| KM20L2-Colon cancer | 0.0 | PANC-1-Pancreatic | 0.0 |
| | | epithelioid ductal carcinoma | |
| NCI-H716-Colon cancer | 0.0 | T24-Bladder carcinma | 0.0 |
| | | (transitional cell) | |
| SW-48-Colon | 0.0 | 5637-Bladder carcinoma | 0.0 |
| adenocarcinoma | | | |
| SW1116-Colon | 0.0 | HT-1197-Bladder carcinoma | 0.0 |
| adenocarcinoma | | | |
| LS 174T-Colon | 0.0 | UM-UC-3-Bladder carcinma | 0.0 |
| adenocarcinoma | | (transitional cell) | |
| SW-948-Colon | 0.0 | A204-Rhabdomyosarcoma | 0.0 |
| adenocarcinoma | | | |
| SW-480-Colon | 0.0 | HT-1080-Fibrosarcoma | 0.0 |
| adenocarcinoma | | | |
| NCI-SNU-5-Gastric | 9.7 | MG-63-Osteosarcoma | 0.0 |
| carcinoma | | | |
| KATO III-Gastric | 0.0 | SK-LMS-1-Leiomyosarcoma | 0.0 |
| carcinoma | | (vulva) | |
| NCI-SNU-16-Gastric | 0.0 | SJRH30-Rhabdomyosarcoma | 0.0 |
| carcinoma | | (met to bone marrow) | |
| NCI-SNU-1-Gastric | 0.0 | A431-Epidermoid carcinoma | 0.0 |
| carcinoma | | | |
| RF-1-Gastric | 0.0 | WM266-4-Melanoma | 0.0 |
| adenocarcinoma | | | |
| RF-48-Gastric | 0.0 | DU 145-Prostate carcinoma | 0.0 |
| adenocarcinoma | | (brain metastasis) | |
| MKN-45-Gastric | 0.0 | MDA-MB-468-Breast | 0.0 |
| carcinoma | | adenocarcinoma | |
| NCI-N87-Gastric | 0.0 | SCC-4-Squamous cell | 0.0 |
| carcinoma | | carcinoma of tongue | |
| OVCAR-5-Ovarian | 0.0 | SCC-9-Squamous cell | 0.0 |
| carcinoma | | carcinoma of tongue | |
| RL95-2-Uterine | 0.0 | SCC-15-Squamous cell | 0.0 |
| carcinoma | | carcinoma of tongue | |
| HelaS3-Cervical | 0.0 | CAL 27-Squamous cell | 0.0 |
| adenocarcinoma | | carcinoma of tongue | |

TABLE ABF
Panel 4D

| | Rel. Exp. (%) | | Rel. Exp. (%) |
|---|---|---|---|
| | Ag2893, Run | | Ag2893, Run |
| Tissue Name | 159633002 | Tissue Name | 159633002 |
| Secondary Th1 act | 0.0 | HUVEC IL-1beta | 0.0 |
| Secondary Th2 act | 0.0 | HUVEC IFN gamma | 0.0 |
| Secondary Tr1 act | 0.0 | HUVEC TNF alpha + IFN | 0.0 |
| | | gamma | |
| Secondary Th1 rest | 0.0 | HUVEC TNF alpha + IL4 | 0.0 |
| Secondary Th2 rest | 1.2 | HUVEC IL-11 | 0.0 |
| Secondary Tr1 rest | 0.0 | Lung Microvascular EC | 0.0 |
| | | none | |
| Primary Th1 act | 0.0 | Lung Microvascular EC | 0.0 |
| | | TNF alpha + IL-1beta | |
| Primary Th2 act | 0.0 | Microvascular Dermal EC | 0.0 |
| | | none | |
| Primary Tr1 act | 0.0 | Microsvasular Dermal EC | 0.0 |
| | | TNF alpha + IL-1beta | |
| Primary Th1 rest | 0.0 | Bronchial epithelium | 0.0 |
| | | TNF alpha + IL1beta | |
| Primary Th2 rest | 0.7 | Small airway epithelium | 1.7 |
| | | none | |
| Primary Tr1 rest | 0.0 | Small airway epithelium | 3.0 |
| | | TNF alpha + IL-1beta | |
| CD45RA CD4 | 0.0 | Coronery artery SMC rest | 0.0 |
| lymphocyte act | | | |
| CD45RO CD4 | 0.3 | Coronery artery SMC | 0.0 |
| lymphocyte act | | TNF alpha + IL-1beta | |
| CD8 lymphocyte act | 0.0 | Astrocytes rest | 4.4 |
| Secondary CD8 | 0.2 | Astrocytes TNF alpha + | 5.8 |
| lymphocyte rest | | IL-1beta | |
| Secondary CD8 | 0.0 | KU-812 (Basophil) rest | 0.0 |
| lymphocyte act | | | |
| CD4 lymphocyte none | 0.0 | KU-812 (Basophil) | 0.0 |
| | | PMA/ionomycin | |
| 2ry Th1/Th2/Tr1_anti- | 0.8 | CCD1106 (Keratinocytes) | 0.0 |
| CD95 CH11 | | none | |
| LAK cells rest | 1.0 | CCD1106 (Keratinocytes) | 0.0 |
| | | TNF alpha + IL-1beta | |
| LAK cells IL-2 | 0.0 | Liver cirrhosis | 12.7 |
| LAK cells IL-2 + IL-12 | 0.4 | Lupus kidney | 18.6 |
| LAK cells IL-2 + IFN | 0.0 | NCI-H292 none | 6.5 |
| gamma | | | |
| LAK cells IL-2 + IL-18 | 0.0 | NCI-H292 IL-4 | 5.6 |
| LAK cells | 0.0 | NCI-H292 IL-9 | 6.8 |
| PMA/ionomycin | | | |
| NK Cells IL-2 rest | 0.0 | NCI-H292 IL-13 | 3.6 |
| Two Way MLR 3 day | 0.0 | NCI-H292 IFN gamma | 2.9 |
| Two Way MLR 5 day | 0.0 | HPAEC none | 0.0 |
| Two Way MLR 7 day | 0.0 | HPAEC TNF alpha + IL- | 0.0 |
| | | 1beta | |
| PBMC rest | 0.3 | Lung fibroblast none | 0.0 |
| PBMC PWM | 0.4 | Lung fibroblast TNF alpha + | 0.0 |
| | | IL-1beta | |
| PBMC PHA-L | 0.0 | Lung fibroblast IL-4 | 0.0 |
| Ramos (B cell) none | 0.0 | Lung fibroblast IL-9 | 0.0 |
| Ramos (B cell) | 0.0 | Lung fibroblast IL-13 | 0.0 |
| ionomycin | | | |
| B lymphocytes PWM | 0.8 | Lung fibroblast IFN | 0.0 |
| | | gamma | |
| B lymphocytes CD40L | 0.6 | Dermal fibroblast | 0.7 |
| and IL-4 | | CCD1070 rest | |
| EOL-1 dbcAMP | 0.0 | Dermal fibroblast | 0.0 |
| | | CCD1070 TNF alpha | |
| EOL-1 dbcAMP | 0.0 | Dermal fibroblast | 0.0 |
| PMA/ionomycin | | CCD1070 IL-1beta | |
| Dendritic cells none | 0.0 | Dermal fibroblast IFN | 0.0 |
| | | gamma | |
| Dendritic cells LPS | 0.0 | Dermal fibroblast IL-4 | 0.0 |
| Dendritic cells anti- | 0.0 | IBD Colitis 2 | 0.0 |
| CD40 | | | |
| Monocytes rest | 0.0 | IBD Crohn's | 1.2 |
| Monocytes LPS | 0.0 | Colon | 0.8 |
| Macrophages rest | 0.0 | Lung | 34.2 |
| Macrophages LPS | 0.0 | Thymus | 100.0 |
| HUVEC none | 0.0 | Kidney | 0.0 |
| HUVEC starved | 0.0 | | |

CNS_neurodegeneration_v1.0 Summary: Ag2893 This panel does not show differential expression of the NOV29c gene in Alzheimer's disease. However, this expression profile confirms the presence of this gene in the brain. Please see Panel 1.3D for a discussion of utility of this gene in the central nervous system.

Panel 1.3D Summary: Ag2893 Two experiments with the same probe and primer set produce results that are in excellent agreement, with highest expression of the NOV29c gene in a renal cancer cell line (CTs=28-30). Significant expression is also seen in a cluster of renal cancer cell lines. Thus, expression of this gene could be used to differentiate between this sample and other samples on this panel and as a marker to detect the presence of renal cancer. Furthermore, therapeutic modulation of the expression or function of this gene may be effective in the treatment of renal cancer.

This gene also is expressed at low, but significant levels in the brain. Expression of the NOV29C gene in the cerebral cortex suggests a role in CNS-specific processes. Homology to the tocopherol-associated protein (TAP) transcription factor suggests a role for NOV29C in tocopherol mediated gene transcription. Tocopherol is an essential vitamin involved in many CNS processes that may be mediated by both its antioxidant properties and ability to regulate gene transcription via NOV29c. Genetic disruption of tocopherol processing results in tocopherol deficiency and CNS disorders such as ataxia and neurodegeneration. Agents that modulate NOV29c may thus have utility in the treatment of ataxia and neurodegenerative diseases.

References:

Yamauchi J, Iwamoto T, Kida S. Masushige S, Yamada K, Esashi T. Tocopherol-associated protein is a ligand-dependent transcriptional activator. Biochem Biophys Res Commun Jul. 13, 2001;285(2):295-9

Vitamin E is a term that encompasses a group of potent, lipid-soluble, chain-breaking antioxidants. Structural analysis reveals that molecules having vitamin E activity include four isomers (alpha, beta, gamma, and delta) of both tocopherols and tocotrienols. Alpha-tocopherol has been shown to have the highest biological vitamin E activity in mammalian tissues based on fetal resorption assays, and it reverses vitamin E deficiency symptoms. Although the molecular functions fulfilled specifically by alpha-tocopherol have yet to be fully described, it is unlikely that they are limited to general antioxidant functions. Here we show the functional characterization of alpha-tocopherol associated protein, TAP, which displays significant sequence similarity to the alpha-tocopherol transfer protein. Ligand competition analysis showed that recombinant TAP binds to alpha-tocopherol but not to other isomers of tocopherols. Using GFP fusion protein expression system, we observed that TAP translocates from cytosol to nuclei in alpha-tocopherol-dependent fashion. Transient transfection experiment showed that TAP activates transcription of the reporter gene in alpha-tocopherol-dependent manner. These results suggest that the biological function of alpha-tocopherol is not only as an antioxidant but also as a transcriptional regulator of gene expression via association with a transcription factor TAP.

Yokota T, Igarashi K, Uchihara T, Jishage K, Tomita H, Inaba A, Li Y, Arita M, Suzuki H, Mizusawa H, Arai H. Delayed-onset ataxia in mice lacking alpha-tocopherol transfer protein: model for neuronal degeneration caused by chronic oxidative stress. Proc Natl Acad Sci USA Dec. 18, 2001;98(26):15185-90

alpha-Tocopherol transfer protein (alpha-TTP) maintains the concentration of serum alpha-tocopherol (vitamin E), one of the most potent fat-soluble antioxidants, by facilitating alpha-tocopherol export from the liver. Mutations of the alpha-TTP gene are linked to ataxia with isolated vitamin E deficiency (AVED). We produced a model mouse of AVED by deleting the alpha-TTP gene, which showed ataxia and retinal degeneration after 1 year of age. Because the brain alpha-TTP functions in maintaining alpha-tocopherol levels in the brain, alpha-tocopherol was completely depleted in the alpha-TTP(−/−) mouse brain, and the neurological phenotype of alpha-TTP(−/−) mice is much more severe than that of wild-type mice when maintained on an alpha-tocopherol-deficient diet. Lipid peroxidation in alpha-TTP(−/−) mice brains showed a significant increase, especially in degenerating neurons. alpha-Tocopherol supplementation suppressed lipid peroxidation and almost completely prevented the development of neurological symptoms. This therapy almost completely corrects the abnormalities in a mouse model of human neurodegenerative disease. Moreover, alpha-TTP(−/−) mice may prove to be excellent animal models of delayed onset, slowly progressive neuronal degeneration caused by chronic oxidative stress.

Panel 2D Summary: Ag2893 Highest expression of the NOV29c gene is seen in a sample derived from a kidney cancer cell line (CT=29.5). In addition, this sample is more highly expressed in kidney cancer than in adjacent normal tissue. Thus, expression of this gene could be used to differentiate between this sample and other samples on this panel and as a marker to detect the presence of kidney cancer. Furthermore, therapeutic modulation of the expression or function of this gene may be effective in the treatment of kidney cancer.

Panel 3D Summary: Ag2893 Expression of the NOV29c gene is detected primarily in samples derived from kidney cancer cell lines (CTs=30). Thus, expression of this gene could be used to differentiate between these samples and other samples on this panel and as a marker to detect the presence of kidney cancer. Furthermore, therapeutic modulation of the expression or function of this gene may be effective in the treatment of kidney cancer.

Panel 4D Summary: Ag2893 The NOV29c transcript is expressed at low but significant levels in the lung and thymus and in lupus kidney and cirrhotic liver. Thus, the transcript or the protein it encodes could be used for detection of these tissues. The expression of this gene suggests that the protein encoded by this transcript may play an important role in the normal homeostasis of the thymus and lung tissues. Therefore, therapeutics designed with the protein encoded by this transcript could be important for modulating T cell development in the thymus and for maintaining or restoring normal function to these lung during inflammation due to diseases such as asthma and emphysema. Additionally, induction of this transcript in other tissues such as the kidney and liver may be detrimental and antagonistic therapies designed with the protein encoded for by this transcript could be important in the treatment of diseases of these tissues.

NOV24b

Expression of gene NOV24b was assessed using the primer-probe set Ag1688, described in Table ACA. Results of the RTQ-PCR runs are shown in Tables ACB, ACC and ACD.

TABLE ACA
Probe Name Ag1688

| | | | Start | |
|---|---|---|---|---|
| Primers | Sequence | Length | Position | SEQ ID NO: |
| Forward | 5′-tcagaagggaatcatgatatcg-3′ | 22 | 577 | 1096 |
| Probe | TET-5′-ccttgataaaactccaggctcctttga-3′-TAMRA | 27 | 550 | 1097 |
| Reverse | 5′-tttggaaggtaggcatattgg-3′ | 21 | 509 | 1098 |

TABLE ACB
Panel 1.3D

| | Rel. Exp. (%) Ag1688, | | Rel. Exp. (%) Ag1688, |
|---|---|---|---|
| Tissue Name | Run 147249266 | Tissue Name | Run 147249266 |
| Liver adenocarcinoma | 0.0 | Kidney (fetal) | 9.2 |
| Pancreas | 6.7 | Renal ca. 786-0 | 0.0 |
| Pancreatic ca. CAPAN 2 | 0.2 | Renal ca. A498 | 1.7 |
| Adrenal gland | 1.8 | Renal ca. RXF 393 | 0.0 |
| Thyroid | 3.8 | Renal ca. ACHN | 0.0 |
| Salivary gland | 1.5 | Renal ca. UO-31 | 0.0 |
| Pituitary gland | 6.1 | Renal ca. TK-10 | 0.0 |
| Brain (fetal) | 0.5 | Liver | 100.0 |
| Brain (whole) | 3.6 | Liver (fetal) | 99.3 |
| Brain (amygdala) | 3.3 | Liver ca. | 0.0 |
| | | (hepatoblast) HepG2 | |
| Brain (cerebellum) | 0.4 | Lung | 1.3 |
| Brain (hippocampus) | 6.2 | Lung (fetal) | 1.8 |
| Brain (substantia nigra) | 1.0 | Lung ca. (small cell) | 0.0 |
| | | LX-1 | |
| Brain (thalamus) | 2.1 | Lung ca. (small cell) | 0.0 |
| | | NCI-H69 | |
| Cerebral Cortex | 6.3 | Lung ca. (s.cell var.) | 0.8 |
| | | SHP-77 | |
| Spinal cord | 3.1 | Lung ca. (large | 0.0 |
| | | cell)NCI-H460 | |
| glio/astro U87-MG | 0.0 | Lung ca. (non-sm. | 0.2 |
| | | cell) A549 | |
| glio/astro U-118-MG | 0.0 | Lung ca. (non-s.cell) | 0.0 |
| | | NCI-H23 | |
| astrocytoma SW1783 | 0.0 | Lung ca. (non-s.cell) | 0.0 |
| | | HOP-62 | |
| neuro*; met SK-N-AS | 0.2 | Lung ca. (non-s.cl) | 0.0 |
| | | NCI-H522 | |
| astrocytoma SF-539 | 0.0 | Lung ca. (squam.) | 0.2 |
| | | SW 900 | |
| astrocytoma SNB-75 | 0.1 | Lung ca. (squam.) | 0.0 |
| | | NCI-H596 | |
| glioma SNB-19 | 0.2 | Mammary gland | 2.9 |
| glioma U251 | 1.2 | Breast ca.* (pl.ef) | 0.0 |
| | | MCF-7 | |
| glioma SF-295 | 0.0 | Breast ca.* (pl.ef) | 0.0 |
| | | MDA-MB-231 | |
| Heart (fetal) | 0.2 | Breast ca.* (pl.ef) | 0.0 |
| | | T47D | |
| Heart | 1.6 | Breast ca. BT-549 | 0.0 |
| Skeletal muscle (fetal) | 0.7 | Breast ca. MDA-N | 0.0 |
| Skeletal muscle | 1.2 | Ovary | 0.0 |
| Bone marrow | 0.5 | Ovarian ca. OVCAR-3 | 0.2 |
| Thymus | 3.2 | Ovarian ca. OVCAR-4 | 0.0 |
| Spleen | 1.0 | Ovarian ca. OVCAR-5 | 0.3 |
| Lymph node | 2.9 | Ovarian ca. OVCAR-8 | 0.0 |
| Colorectal | 0.8 | Ovarian ca. IGROV-1 | 0.0 |
| Stomach | 3.3 | Ovarian ca.* (ascites) | 1.0 |
| | | SK-OV-3 | |
| Small intestine | 6.2 | Uterus | 1.4 |
| Colon ca. SW480 | 0.0 | Placenta | 0.4 |
| Colon ca.* | 0.0 | Prostate | 1.0 |
| SW620(SW480 met) | | | |
| Colon ca. HT29 | 0.0 | Prostate ca.* (bone | 0.0 |
| | | met)PC-3 | |
| Colon ca. HCT-116 | 0.0 | Testis | 6.1 |
| Colon ca. CaCo-2 | 0.2 | Melanoma | 0.4 |
| | | Hs688(A).T | |
| Colon ca. | 0.0 | Melanoma* (met) | 0.9 |
| tissue(ODO3866) | | Hs688(B).T | |
| Colon ca. HCC-2998 | 0.2 | Melanoma UACC-62 | 0.0 |
| Gastric ca.* (liver met) | 4.4 | Melanoma M14 | 0.0 |
| NCI-N87 | | | |
| Bladder | 3.1 | Melanoma LOX | 0.0 |
| | | IMVI | |
| Trachea | 3.0 | Melanoma* (met) | 0.0 |
| | | SK-MEL-5 | |
| Kidney | 6.8 | Adipose | 0.5 |

TABLE ACC
Panel 2D

| | Rel. Exp. (%) | | Rel. Exp. (%) |
|---|---|---|---|
| | Ag1688, Run | | Ag1688, Run |
| Tissue Name | 162646059 | Tissue Name | 162646059 |
| Normal Colon | 1.7 | Kidney Margin | 0.7 |
| | | 8120608 | |
| CC Well to Mod Diff | 0.0 | Kidney Cancer | 0.0 |
| (ODO3866) | | 8120613 | |
| CC Margin (ODO3866) | 0.2 | Kidney Margin | 0.5 |
| | | 8120614 | |
| CC Gr.2 rectosigmoid | 0.2 | Kidney Cancer | 0.2 |
| (ODO3868) | | 9010320 | |
| CC Margin (ODO3868) | 0.1 | Kidney Margin | 1.0 |
| | | 9010321 | |
| CC Mod Diff (ODO3920) | 0.1 | Normal Uterus | 0.2 |
| CC Margin (ODO3920) | 0.9 | Uterus Cancer 064011 | 0.8 |
| CC Gr.2 ascend colon | 0.1 | Normal Thyroid | 0.9 |
| (ODO3921) | | | |
| CC Margin (ODO3921) | 0.1 | Thyroid Cancer | 0.2 |
| | | 064010 | |
| CC from Partial | 4.7 | Thyroid Cancer | 0.5 |
| Hepatectomy (ODO4309) | | A302152 | |
| Mets | | | |
| Liver Margin (ODO4309) | 100.0 | Thyroid Margin | 1.0 |
| | | A302153 | |
| Colon mets to lung | 0.1 | Normal Breast | 0.3 |
| (OD04451-01) | | | |
| Lung Margin (OD04451- | 0.1 | Breast Cancer | 0.1 |
| 02) | | (OD04566) | |
| Normal Prostate 6546-1 | 2.1 | Breast Cancer | 0.1 |
| | | (OD04590-01) | |
| Prostate Cancer | 0.6 | Breast Cancer Mets | 0.4 |
| (OD04410) | | (OD04590-03) | |
| Prostate Margin | 0.5 | Breast Cancer | 0.9 |
| (OD04410) | | Metastasis | |
| | | (OD04655-05) | |
| Prostate Cancer | 1.1 | Breast Cancer 064006 | 0.6 |
| (OD04720-01) | | | |
| Prostate Margin | 1.6 | Breast Cancer 1024 | 1.2 |
| (OD04720-02) | | | |
| Normal Lung 061010 | 2.0 | Breast Cancer | 0.1 |
| | | 9100266 | |
| Lung Met to Muscle | 0.0 | Breast Margin | 0.1 |
| (ODO4286) | | 9100265 | |
| Muscle Margin | 0.2 | Breast Cancer | 0.3 |
| (ODO4286) | | A209073 | |
| Lung Malignant Cancer | 0.1 | Breast Margin | 0.3 |
| (OD03126) | | A2090734 | |
| Lung Margin (OD03126) | 0.5 | Normal Liver | 69.7 |
| Lung Cancer (OD04404) | 0.1 | Liver Cancer 064003 | 13.7 |
| Lung Margin (OD04404) | 0.2 | Liver Cancer 1025 | 18.0 |
| Lung Cancer (OD04565) | 0.0 | Liver Cancer 1026 | 1.2 |
| Lung Margin (OD04565) | 0.1 | Liver Cancer 6004-T | 22.2 |
| Lung Cancer (OD04237- | 0.1 | Liver Tissue 6004-N | 1.0 |
| 01) | | | |
| Lung Margin (OD04237- | 0.4 | Liver Cancer 6005-T | 1.9 |
| 02) | | | |
| Ocular Mel Met to Liver | 0.1 | Liver Tissue 6005-N | 4.2 |
| (ODO4310) | | | |
| LiverMargin (ODO4310) | 77.4 | Normal Bladder | 2.7 |
| Melanoma Mets to Lung | 0.0 | Bladder Cancer 1023 | 0.0 |
| (OD04321) | | | |
| Lung Margin (OD04321) | 0.1 | Bladder Cancer | 0.2 |
| | | A302173 | |
| Normal Kidney | 12.9 | Bladder Cancer | 0.1 |
| | | (OD04718-01) | |
| Kidney Ca, Nuclear grade | 3.8 | Bladder Normal | 0.5 |
| 2 (OD04338) | | Adjacent (OD04718- | |
| | | 03) | |
| Kidney Margin | 1.6 | Normal Ovary | 0.0 |
| (OD04338) | | | |
| Kidney Ca Nuclear grade | 2.8 | Ovarian Cancer | 0.1 |
| 1/2 (OD04339) | | 064008 | |
| Kidney Margin | 9.3 | Ovarian Cancer | 0.2 |
| (OD04339) | | (OD04768-07) | |
| Kidney Ca, Clear cell | 1.4 | Ovary Margin | 0.1 |
| type (OD04340) | | (OD04768-08) | |
| Kidney Margin | 4.1 | Normal Stomach | 0.3 |
| (OD04340) | | | |
| Kidney Ca, Nuclear grade | 0.1 | Gastric Cancer | 0.1 |
| 3 (OD04348) | | 9060358 | |
| Kidney Margin | 3.8 | Stomach Margin | 0.0 |
| (OD04348) | | 9060359 | |
| Kidney Cancer | 0.2 | Gastric Cancer | 0.2 |
| (OD04622-01) | | 9060395 | |
| Kidney Margin | 0.7 | Stomach Margin | 0.3 |
| (OD04622-03) | | 9060394 | |
| Kidney Cancer | 0.2 | Gastric Cancer | 0.3 |
| (OD04450-01) | | 9060397 | |
| Kidney Margin | 2.6 | Stomach Margin 9060396 | 0.0 |
| (OD04450-03) | | | |
| Kidney Cancer 8120607 | 0.0 | Gastric Cancer | 1.1 |
| | | 064005 | |

TABLE ACD
Panel 5 Islet

| | Rel. Exp. (%) | | Rel. Exp. (%) |
|---|---|---|---|
| | Ag1688, Run | | Ag1688, Run |
| Tissue Name | 226587524 | Tissue Name | 226587524 |
| 97457_Patient- | 41.2 | 94709_Donor 2 AM - A_adipose | 0.0 |
| 02go_adipose | | | |
| 97476_Patient- | 9.9 | 94710_Donor 2 AM - B_adipose | 0.0 |
| 07sk_skeletal muscle | | | |
| 97477_Patient- | 8.1 | 94711_Donor 2 AM - C_adipose | 0.0 |
| 07ut_uterus | | | |
| 97478_Patient- | 0.0 | 94712_Donor 2 AD - A_adipose | 11.4 |
| 07pl_placenta | | | |
| 99167_Bayer Patient 1 | 84.7 | 94713_Donor 2 AD - B_adipose | 0.0 |
| 97482_Patient- | 2.4 | 94714_Donor 2 AD - C_adipose | 29.1 |
| 08ut_uterus | | | |
| 97483_Patient- | 0.0 | 94742_Donor 3 U - | 19.2 |
| 08pl_placenta | | A_Mesenchymal Stem Cells | |
| 97486_Patient- | 8.0 | 94743_Donor 3 U - | 0.0 |
| 09sk_skeletal muscle | | B_Mesenchymal Stem Cells | |
| 97487_Patient- | 9.6 | 94730_Donor 3 AM - A_adipose | 15.0 |
| 09ut_uterus | | | |
| 97488_Patient- | 0.0 | 94731_Donor 3 AM - B_adipose | 37.9 |
| 09pl_placenta | | | |
| 97492_Patient- | 0.0 | 94732_Donor 3 AM - C_adipose | 0.0 |
| 10ut_uterus | | | |
| 97493_Patient- | 0.0 | 94733_Donor 3 AD - A_adipose | 39.2 |
| 10pl_placenta | | | |
| 97495_Patient- | 0.0 | 94734_Donor 3 AD - B_adipose | 11.4 |
| 11go_adipose | | | |
| 97496_Patient- | 52.9 | 94735_Donor 3 AD - C_adipose | 34.4 |
| 11sk_skeletal muscle | | | |
| 97497_Patient- | 35.8 | 77138_Liver_HepG2untreated | 8.4 |
| 11ut_uterus | | | |
| 97498_Patient- | 10.5 | 73556_Heart_Cardiac stromal | 0.0 |
| 11pl_placenta | | cells (primary) | |
| 97500_Patient- | 0.0 | 81735_Small Intestine | 100.0 |
| 12go_adipose | | | |
| 97501_Patient- | 35.4 | 72409_Kidney_Proximal | 9.9 |
| 12sk_skeletal muscle | | Convoluted Tubule | |
| 97502_Patient- | 20.7 | 82685_Small intestine_Duodenum | 70.2 |
| 12ut_uterus | | | |
| 97503_Patient- | 0.0 | 90650_Adrenal_Adrenocortical | 25.5 |
| 12pl_placenta | | adenoma | |
| 94721_Donor 2 U - | 0.0 | 72410_Kidney_HRCE | 10.4 |
| A_Mesenchymal Stem | | | |
| Cells | | | |
| 94722_Donor 2 U - | 0.0 | 72411_Kidney_HRE | 7.2 |
| B_Mesenchymal Stem | | | |
| Cells | | | |
| 94723_Donor 2 U - | 0.0 | 73139_Uterus_Uterine smooth | 0.0 |
| C_Mesenchymal Stem | | muscle cells | |
| Cells | | | |

Panel 1.3D Summary: Ag1688 Expression of the NOV24b gene, a plasma kallikrein homolog, is significantly higher in liver (CTs=28) than in any other sample on this panel. Thus, expression of this gene could be used as a marker of liver tissue. Plasma kallikrein is a serine protease that, among other roles, plays a part in blood coagulation, fibrinolysis, and complement activation and has been implicated in adipose differentiation by remodelling of the fibronectin-rich ECM of preadipocytes. Therefore, an antagonist to this gene product may be beneficial in the treatment of obesity.

References:

Hoover-Plow J, Yuen L. Plasminogen binding is increased with adipocyte differentiation. Biochem.Biophys.Res.Commun. (2001) 284, 389-394

The purpose of this study was to examine the role of the plasminogen system in the development of adipose tissue. Plasminogen binding capacity was determined in differentiated and undifferentiated cells from adipose tissue of plasminogen deficient mice and 3T3 cells, a well-characterized tissue culture model. In 3T3 cells, plasminogen binding was fivefold higher in differentiated cells compared to the undifferentiated cells. Inhibition of binding by carboxyl-terminal lysine analogs was similar for the differentiated and undifferentiated cells with tranexamic acid>EACA>lysine. The binding of plasminogen was concentration-dependent and approaches saturation in the both cell types. The number of plasminogen binding sites was tenfold higher in the differentiated compared to the undifferentiated cells. In isolated mature fat cells and stromal cell cultures from mouse adipose tissue, plasminogen binding was also higher in the differentiated mature fat cells and differentiated stromal cells compared to undifferentiated stromal cells. Plasminogen binding was elevated in the differentiated cells from the Plg−/− mice compared to cells from the WT mice. These results suggest that the plasminogen system plays an important role in adipose tissue development. Copyright 2001 Academic Press.

PMID: 11394891

Selvarajan S, Lund L R, Takeuchi T, Craik C S, Werb Z. A plasma kallikrein-dependent plasminogen cascade required for adipocyte differentiation. Nature Cell Biol. (2001) 3, 267-275.

Here we show that plasma kallikrein (PKal) mediates a plasminogen (Plg) cascade in adipocyte differentiation. Ecotin, an inhibitor of serine proteases, inhibits cell-shape change, adipocyte-specific gene expression, and lipid accumulation during adipogenesis in culture. Deficiency of Plg, but not of urokinase or tissue-type plasminogen activator, suppresses adipogenesis during differentiation of 3T3-L1 cells and mammary-gland involution. PKal, which is inhibited by ecotin, is required for adipose conversion, Plg activation and 3T3-L1 differentiation. Human plasma lacking PKal does not support differentiation of 3T3-L1 cells. PKal is therefore a physiological regulator that acts in the Plg cascade during adipogenesis. We propose that the Plg cascade fosters adipocyte differentiation by degradation of the fibronectin-rich preadipocyte stromal matrix.

PMID: 11231576

Panel 2D Summary: Ag1688 The expression of the NOV24b gene appears to be highest in a sample derived from a sample of normal liver tissue adjacent to a metastatic colon cancer CT=26.2). In addition, there is substantial expression in other samples of normal liver, and to a much lesser degree, malignant liver tissue. This liver specific expression is consistent with the expression seen in Panel 1.3D. Thus, the expression of this gene could be used to distinguish liver derived tissue from the other samples in the panel, and more specifically the expression of this gene could be used to distinguish normal liver from malignant liver tissue. Moreover, therapeutic modulation of this gene, through the use of small molecule drugs, protein therapeutics or antibodies might be of benefit in the treatment of liver cancer.

Panel 5 Islet Summary: Ag1688 Expression of the NOV24b gene is limited to pancreatic islets and small intestines. Please see Panel 1.3 for discussion of utility of this gene in metabolic disease.

NOV30

Expression of gene NOV30 was assessed using the primer-probe set Ag2894, described in Table ADA. Results of the RTQ-PCR runs are shown in Tables ADB, ADC and ADD.

TABLE ADA
Probe Name Ag2894

| | | | Start | |
|---|---|---|---|---|
| Primers | Sequence | Length | Position | SEQ ID NO: |
| Forward | 5′-cctgagtcaatccaagaaactg-3′ | 22 | 94 | 1099 |
| Probe | TET-5′-aggtcatcaacccaggaccgcctag-3′-TAMRA | 25 | 116 | 1100 |
| Reverse | 5′-tccagtagggatctggagaagt-3′ | 22 | 149 | 1101 |

TABLE ADB
Panel 1.3D

| | Rel. Exp. (%) Ag2894, | | Rel. Exp. (%) Ag2894, |
|---|---|---|---|
| Tissue Name | Run 160968507 | Tissue Name | Run 160968507 |
| Liver adenocarcinoma | 0.0 | Kidney (fetal) | 0.0 |
| Pancreas | 0.0 | Renal ca. 786-0 | 0.0 |
| Pancreatic ca. CAPAN 2 | 0.0 | Renal ca. A498 | 0.0 |
| Adrenal gland | 0.0 | Renal ca. RXF 393 | 0.0 |
| Thyroid | 0.0 | Renal ca. ACHN | 50.3 |
| Salivary gland | 0.0 | Renal ca. UO-31 | 0.0 |
| Pituitary gland | 0.0 | Renal ca. TK-10 | 0.0 |
| Brain (fetal) | 0.0 | Liver | 8.1 |
| Brain (whole) | 0.0 | Liver (fetal) | 6.4 |
| Brain (amygdala) | 0.0 | Liver ca. | 0.0 |
| | | (hepatoblast) HepG2 | |
| Brain (cerebellum) | 0.0 | Lung | 0.0 |
| Brain (hippocampus) | 0.0 | Lung (fetal) | 6.7 |
| Brain (substantia nigra) | 0.0 | Lung ca. (small cell) | 0.0 |
| | | LX-1 | |
| Brain (thalamus) | 0.0 | Lung ca. (small cell) | 0.0 |
| | | NCI-H69 | |
| Cerebral Cortex | 0.0 | Lung ca. (s.cell var.) | 0.0 |
| | | SHP-77 | |
| Spinal cord | 0.0 | Lung ca. (large | 0.0 |
| | | cell)NCI-H460 | |
| glio/astro U87-MG | 0.0 | Lung ca. (non-sm. | 9.7 |
| | | cell) A549 | |
| glio/astro U-118-MG | 0.0 | Lung ca. (non-s.cell) | 19.8 |
| | | NCI-H23 | |
| astrocytoma SW1783 | 0.0 | Lung ca. (non-s.cell) | 0.0 |
| | | HOP-62 | |
| neuro*; met SK-N-AS | 0.0 | Lung ca. (non-s.cl) | 0.0 |
| | | NCI-H522 | |
| astrocytoma SF-539 | 3.4 | Lung ca. (squam.) | 0.0 |
| | | SW 900 | |
| astrocytoma SNB-75 | 0.0 | Lung ca. (squam.) | 0.0 |
| | | NCI-H596 | |
| glioma SNB-19 | 0.0 | Mammary gland | 0.0 |
| glioma U251 | 0.0 | Breast ca.* (pl.ef) | 0.0 |
| | | MCF-7 | |
| glioma SF-295 | 0.0 | Breast ca.* (pl.ef) | 3.3 |
| | | MDA-MB-231 | |
| Heart (fetal) | 3.5 | Breast ca.* (pl.ef) | 0.0 |
| | | T47D | |
| Heart | 0.0 | Breast ca. BT-549 | 0.0 |
| Skeletal muscle (fetal) | 1.2 | Breast ca. MDA-N | 0.0 |
| Skeletal muscle | 0.0 | Ovary | 6.5 |
| Bone marrow | 0.0 | Ovarian ca. OVCAR-3 | 0.0 |
| Thymus | 0.0 | Ovarian ca. OVCAR-4 | 0.0 |
| Spleen | 0.0 | Ovarian ca. OVCAR-5 | 0.0 |
| Lymph node | 0.0 | Ovarian ca. OVCAR-8 | 0.0 |
| Colorectal | 0.0 | Ovarian ca. IGROV-1 | 0.0 |
| Stomach | 0.0 | Ovarian ca.* (ascites) | 0.0 |
| | | SK-OV-3 | |
| Small intestine | 0.0 | Uterus | 0.0 |
| Colon ca. SW480 | 0.0 | Placenta | 0.0 |
| Colon ca.* | 0.0 | Prostate | 0.0 |
| SW620(SW480 met) | | | |
| Colon ca. HT29 | 0.0 | Prostate ca.* (bone | 0.0 |
| | | met)PC-3 | |
| Colon ca. HCT-116 | 0.0 | Testis | 100.0 |
| Colon ca. CaCo-2 | 0.0 | Melanoma | 0.0 |
| | | Hs688(A).T | |
| Colon ca. | 0.0 | Melanoma* (met) | 0.0 |
| tissue(ODO3866) | | Hs688(B).T | |
| Colon ca. HCC-2998 | 0.0 | Melanoma UACC-62 | 0.0 |
| Gastric ca.* (liver met) | 5.7 | Melanoma M14 | 0.0 |
| NCI-N87 | | | |
| Bladder | 0.0 | Melanoma LOX | 0.0 |
| | | IMVI | |
| Trachea | 14.0 | Melanoma* (met) | 0.0 |
| | | SK-MEL-5 | |
| Kidney | 0.0 | Adipose | 4.2 |

TABLE ADC
Panel 2D

| | Rel. Exp. (%) | | Rel. Exp. (%) |
|---|---|---|---|
| | Ag2894, Run | | Ag2894, Run |
| Tissue Name | 160966709 | Tissue Name | 160966709 |
| Normal Colon | 0.0 | Kidney Margin | 0.0 |
| | | 8120608 | |
| CC Well to Mod Diff | 0.0 | Kidney Cancer | 0.0 |
| (ODO3866) | | 8120613 | |
| CC Margin (ODO3866) | 0.0 | Kidney Margin | 0.0 |
| | | 8120614 | |
| CC Gr.2 rectosigmoid | 0.0 | Kidney Cancer | 0.0 |
| (ODO3868) | | 9010320 | |
| CC Margin (ODO3868) | 0.0 | Kidney Margin | 0.0 |
| | | 9010321 | |
| CC Mod Diff (ODO3920) | 0.0 | Normal Uterus | 0.0 |
| CC Margin (ODO3920) | 0.0 | Uterus Cancer 064011 | 0.0 |
| CC Gr.2 ascend colon | 6.4 | Normal Thyroid | 0.0 |
| (ODO3921) | | | |
| CC Margin (ODO3921) | 0.0 | Thyroid Cancer | 0.0 |
| | | 064010 | |
| CC from Partial | 3.0 | Thyroid Cancer | 0.0 |
| Hepatectomy (ODO4309) | | A302152 | |
| Mets | | | |
| Liver Margin (ODO4309) | 40.3 | Thyroid Margin | 0.0 |
| | | A302153 | |
| Colon mets to lung | 0.0 | Normal Breast | 0.0 |
| (OD04451-01) | | | |
| Lung Margin (OD04451- | 0.0 | Breast Cancer | 0.0 |
| 02) | | (OD04566) | |
| Normal Prostate 6546-1 | 0.0 | Breast Cancer | 0.0 |
| | | (OD04590-01) | |
| Prostate Cancer | 0.0 | Breast Cancer Mets | 0.0 |
| (OD04410) | | (OD04590-03) | |
| Prostate Margin | 0.0 | Breast Cancer | 0.0 |
| (OD04410) | | Metastasis | |
| | | (OD04655-05) | |
| Prostate Cancer | 0.0 | Breast Cancer 064006 | 0.0 |
| (OD04720-01) | | | |
| Prostate Margin | 0.0 | Breast Cancer 1024 | 0.0 |
| (OD04720-02) | | | |
| Normal Lung 061010 | 9.7 | Breast Cancer | 0.0 |
| | | 9100266 | |
| Lung Met to Muscle | 3.9 | Breast Margin | 0.0 |
| (ODO4286) | | 9100265 | |
| Muscle Margin | 0.0 | Breast Cancer | 0.0 |
| (ODO4286) | | A209073 | |
| Lung Malignant Cancer | 4.4 | Breast Margin | 0.0 |
| (OD03126) | | A2090734 | |
| Lung Margin (OD03126) | 25.0 | Normal Liver | 100.0 |
| Lung Cancer (OD04404) | 12.5 | Liver Cancer 064003 | 5.3 |
| Lung Margin (OD04404) | 15.0 | Liver Cancer 1025 | 20.2 |
| Lung Cancer (OD04565) | 0.0 | Liver Cancer 1026 | 12.7 |
| Lung Margin (OD04565) | 8.1 | Liver Cancer 6004-T | 13.0 |
| Lung Cancer (OD04237- | 2.0 | Liver Tissue 6004-N | 0.0 |
| 01) | | | |
| Lung Margin (OD04237- | 0.0 | Liver Cancer 6005-T | 0.0 |
| 02) | | | |
| Ocular Mel Met to Liver | 0.0 | Liver Tissue 6005-N | 0.0 |
| (ODO4310) | | | |
| Liver Margin (ODO4310) | 19.3 | Normal Bladder | 0.0 |
| Melanoma Mets to Lung | 0.0 | Bladder Cancer 1023 | 0.0 |
| (OD04321) | | | |
| Lung Margin (OD04321) | 16.2 | Bladder Cancer | 15.7 |
| | | A302173 | |
| Normal Kidney | 0.0 | Bladder Cancer | 0.0 |
| | | (OD04718-01) | |
| Kidney Ca, Nuclear grade | 2.9 | Bladder Normal | 0.0 |
| 2 (OD04338) | | Adjacent (OD04718- | |
| | | 03) | |
| Kidney Margin | 0.0 | Normal Ovary | 0.0 |
| (OD04338) | | | |
| Kidney Ca Nuclear grade | 3.7 | Ovarian Cancer | 0.0 |
| 1/2 (OD04339) | | 064008 | |
| Kidney Margin | 0.0 | Ovarian Cancer | 0.0 |
| (OD04339) | | (OD04768-07) | |
| Kidney Ca, Clear cell | 0.0 | Ovary Margin | 0.0 |
| type (OD04340) | | (OD04768-08) | |
| Kidney Margin | 0.0 | Normal Stomach | 4.1 |
| (OD04340) | | | |
| Kidney Ca, Nuclear grade | 0.0 | Gastric Cancer | 0.0 |
| 3 (OD04348) | | 9060358 | |
| Kidney Margin | 0.0 | Stomach Margin | 0.0 |
| (OD04348) | | 9060359 | |
| Kidney Cancer | 0.0 | Gastric Cancer | 0.0 |
| (OD04622-01) | | 9060395 | |
| Kidney Margin | 0.0 | Stomach Margin | 0.0 |
| (OD04622-03) | | 9060394 | |
| Kidney Cancer | 0.0 | Gastric Cancer | 0.0 |
| (OD04450-01) | | 9060397 | |
| Kidney Margin | 0.0 | Stomach Margin | 0.0 |
| (OD04450-03) | | 9060396 | |
| Kidney Cancer 8120607 | 0.0 | Gastric Cancer | 0.0 |
| | | 064005 | |

TABLE ADD
Panel 4D

| | Rel. Exp. (%) | | Rel. Exp. (%) |
|---|---|---|---|
| | Ag2894, Run | | Ag2894, Run |
| Tissue Name | 164033148 | Tissue Name | 164033148 |
| Secondary Th1 act | 0.0 | HUVEC IL-1beta | 0.0 |
| Secondary Th2 act | 0.0 | HUVEC IFN gamma | 0.0 |
| Secondary Tr1 act | 0.0 | HUVEC TNF alpha + IFN | 0.0 |
| | | gamma | |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC | 0.0 |
| | | none | |
| Primary Th1 act | 0.0 | Lung Microvascular EC | 0.0 |
| | | TNF alpha + IL-1beta | |
| Primary Th2 act | 0.0 | Microvascular Dermal EC | 0.0 |
| | | none | |
| Primary Tr1 act | 0.0 | Microsvasular Dermal EC | 0.0 |
| | | TNF alpha + IL-1beta | |
| Primary Th1 rest | 0.0 | Bronchial epithelium | 0.0 |
| | | TNF alpha + IL1beta | |
| Primary Th2 rest | 0.0 | Small airway epithelium | 0.0 |
| | | none | |
| Primary Tr1 rest | 0.0 | Small airway epithelium | 0.0 |
| | | TNF alpha + IL-1beta | |
| CD45RA CD4 | 0.0 | Coronery artery SMC rest | 0.0 |
| lymphocyte act | | | |
| CD45RO CD4 | 17.3 | Coronery artery SMC | 0.0 |
| lymphocyte act | | TNF alpha + IL-1beta | |
| CD8 lymphocyte act | 0.0 | Astrocytes rest | 0.0 |
| Secondary CD8 | 0.0 | Astrocytes TNF alpha + | 0.0 |
| lymphocyte rest | | IL-1beta | |
| Secondary CD8 | 0.0 | KU-812 (Basophil) rest | 0.0 |
| lymphocyte act | | | |
| CD4 lymphocyte none | 0.0 | KU-812 (Basophil) | 0.0 |
| | | PMA/ionomycin | |
| 2ry Th1/Th2/Tr1_anti- | 0.0 | CCD1106 (Keratinocytes) | 0.0 |
| CD95 CH11 | | none | |
| LAK cells rest | 0.0 | CCD1106 (Keratinocytes) | 0.0 |
| | | TNF alpha + IL-1beta | |
| LAK cells IL-2 | 0.0 | Liver cirrhosis | 100.0 |
| LAK cells IL-2 + IL-12 | 0.0 | Lupus kidney | 0.0 |
| LAK cells IL-2 + IFN | 0.0 | NCI-H292 none | 0.0 |
| gamma | | | |
| LAK cells IL-2 + IL-18 | 0.0 | NCI-H292 IL-4 | 0.0 |
| LAK cells | 0.0 | NCI-H292 IL-9 | 0.0 |
| PMA/ionomycin | | | |
| NK Cells IL-2 rest | 0.0 | NCI-H292 IL-13 | 0.0 |
| Two Way MLR 3 day | 0.0 | NCI-H292 IFN gamma | 0.0 |
| Two Way MLR 5 day | 0.0 | HPAEC none | 0.0 |
| Two Way MLR 7 day | 0.0 | HPAEC TNF alpha + IL- | 0.0 |
| | | 1beta | |
| PBMC rest | 0.0 | Lung fibroblast none | 0.0 |
| PBMC PWM | 0.0 | Lung fibroblast TNF alpha + | 0.0 |
| | | IL-1beta | |
| PBMC PHA-L | 13.8 | Lung fibroblast IL-4 | 0.0 |
| Ramos (B cell) none | 0.0 | Lung fibroblast IL-9 | 0.0 |
| Ramos (B cell) | 0.0 | Lung fibroblast IL-13 | 0.0 |
| ionomycin | | | |
| B lymphocytes PWM | 0.0 | Lung fibroblast IFN | 0.0 |
| | | gamma | |
| B lymphocytes CD40L | 0.0 | Dermal fibroblast | 0.0 |
| and IL-4 | | CCD1070 rest | |
| EOL-1 dbcAMP | 0.0 | Dermal fibroblast | 0.0 |
| | | CCD1070 TNF alpha | |
| EOL-1 dbcAMP | 0.0 | Dermal fibroblast | 0.0 |
| PMA/ionomycin | | CCD1070 IL-1beta | |
| Dendritic cells none | 0.0 | Dermal fibroblast IFN | 0.0 |
| | | gamma | |
| Dendritic cells LPS | 0.0 | Dermal fibroblast IL-4 | 0.0 |
| Dendritic cells anti- | 0.0 | IBD Colitis 2 | 7.6 |
| CD40 | | | |
| Monocytes rest | 0.0 | IBD Crohn's | 0.0 |
| Monocytes LPS | 0.0 | Colon | 23.0 |
| Macrophages rest | 0.0 | Lung | 13.8 |
| Macrophages LPS | 0.0 | Thymus | 12.8 |
| HUVEC none | 0.0 | Kidney | 0.0 |
| HUVEC starved | 0.0 | | |

Panel 1.3D Summary: Ag2894 Expression of the NOV30 gene is restricted to the testis and a renal cancer cell line(CTs=33-35). Thus, expression of this gene could be used to differentiate these samples from other samples on this panel and as a marker for testis tissue and renal cancer.

Panel 2D Summary: Ag2894 Expression of the NOV30 gene is restricted to normal liver tissue (CTs=33-35). This gene enodes a ryudocan homolog. Ryudocan is a cell-surface heparan sulfate proteoglycan, which is involved in regulation of blood coagulation, among other biological functions. Thus, based on its expression profile, expression of this gene could be used to identify liver tissue and to differentiate between normal and malignant liver. Furthermore, this gene product may be involved in normal homeostasis of the liver. Thus, therapeutic modulation of the expression or function of this gene product may be effective in the treatment of liver disease and liver cancer.

References:

Kojima T, Inazawa J, Takamatsu J, Rosenberg R D, Saito H. Human ryudocan core protein: molecular cloning and characterization of the cDNA, and chromosomal localization of the gene. Biochem Biophys Res Commun Feb. 15, 1993;190(3):814-22

We have isolated a series of overlapping cDNA clones encoding a 2,628 bp transcript, which potentially codes for a 198 amino acid protein with predicted molecular mass of 21,641 daltons, for the human ryudocan core protein. The deduced core proteins of the human and the rat ryudocan have high structural conservation, particularly in the NH2 and COOH terminus regions of the putative mature core protein, including the combined transmembrane/cytoplasmic domains with conserved positions of all 4 tyrosine groups and 3 conserved glycosaminoglycan chain attachment regions, which might serve important roles for biological function of ryudocan. A major 2.7 kb transcript was detected in all tissues tested, with relatively high levels of expression observed in mRNA from lung, liver, skeletal muscle and kidney. A minor 1.9 kb transcript was also observed in some of tissues, which would be caused by alternative polyadenylation. Human ryudocan gene has been localized on the chromosome 20q12 by fluorescence in situ hybridization.

PMID: 7916598

Kojima T. Molecular biology of ryudocan, an endothelial heparan sulfate proteoglycan. Semin Thromb Hemost 2000;26(1):67-73

Ryudocan is a type I integral membrane heparan sulfate proteoglycan, which was originally cloned from rat microvascular endothelial cells. We have cloned the cDNA of rat ryudocan. The deduced amino acids of ryudocan has homologous transmembrane and intracellular domains with syndecan but very distinct extracellular regions. We also cloned the human ryudocan cDNA, of which the gene localizes on the chromosome 20q12. To better understand the regulation of ryudocan expression, we have determined the structural organization of the human ryudocan gene. The human ryudocan gene extends approximately 24 kb and is divided into five exons that appear conserved in syndecan family members. The 5′-flanking sequences of the human ryudocan gene contain a variety of potential binding sites for transcription factors and are capable of functioning as a promoter. We purified human ryudocan and evaluated its interactions with several extracellular ligands. It was found that basic fibroblast growth factor (bFGF), midkine, and tissue factor pathway inhibitor exhibited significant ryudocan bindings. Heparitinase, but not chondroitin ABC lyase treatment, destroyed those ryudocan bindings; thus, the heparan sulfate chains of ryudocan appear to be responsible for those bindings. Immunohistochemical analysis revealed that ryudocan is expressed in peripheral nerve tissues, fibrous connective tissues, and placental trophoblasts. These findings suggest that ryudocan may possess multiple biologic functions, such as bFGF modulation, neurite growth promotion, and anticoagulation, via heparan sulfate-binding effectors in the cellular microenvironment.

PMID: 10805285

Panel 4D Summary: Ag2894 Expression of the NOV30 gene is restricted to a sample derived from liver cirrhosis. This gene is also expressed in normal liver in panel 2D. This expression suggests that this protein product, a ryudocan homolog, is essential to liver function. Thus, expression of this gene could be used as a marker for liver derived tissue. Furthermore, therapeutic modulation of the expression or function of this gene product may be effective in the treatment of diseases that affect the liver, including liver cirrhosis.

NOV31

Expression of gene NOV31 was assessed using the primer-probe set Ag2922, described in Table AEA. Results of the RTQ-PCR runs are shown in Table AEB.

TABLE AEA

Probe Name Ag2922

| | | | Start | |
|---|---|---|---|---|
| Primers | Sequences | Length | Position | SEQ ID NO: |
| Forward | 5′-cttcaggctgtgggtcatc-3′ | 19 | 407 | 1102 |
| Probe | TET-5′-caagccctactgctcccagtccag-3′- | 24 | 447 | 1103 |
| | TAMRA | | | |
| Reverse | 5′-cagcaacagggcttacaaca-3′ | 20 | 472 | 1104 |

TABLE AEB
Panel 4.1D

| | Rel. Exp. (%) | | Rel. Exp. (%) |
|---|---|---|---|
| | Ag2922, Run | | Ag2922, Run |
| Tissue Name | 171619741 | Tissue Name | 171619741 |
| Secondary Th1 act | 0.0 | HUVEC IL-1beta | 1.0 |
| Secondary Th2 act | 0.0 | HUVEC IFN gamma | 1.3 |
| Secondary Tr1 act | 0.0 | HUVEC TNF alpha + IFN | 0.0 |
| | | gamma | |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.6 | Lung Microvascular EC | 3.2 |
| | | none | |
| Primary Th1 act | 0.0 | Lung Microvascular EC | 1.9 |
| | | TNF alpha + IL-1beta | |
| Primary Th2 act | 0.4 | Microvascular Dermal EC | 0.0 |
| | | none | |
| Primary Tr1 act | 0.0 | Microsvasular Dermal EC | 0.0 |
| | | TNF alpha + IL-1beta | |
| Primary Th1 rest | 1.1 | Bronchial epithelium | 0.0 |
| | | TNF alpha + IL1beta | |
| Primary Th2 rest | 1.3 | Small airway epithelium | 0.0 |
| | | none | |
| Primary Tr1 rest | 0.7 | Small airway epithelium | 0.6 |
| | | TNF alpha + IL-1beta | |
| CD45RA CD4 | 0.0 | Coronery artery SMC rest | 1.8 |
| lymphocyte act | | | |
| CD45RO CD4 | 1.2 | Coronery artery SMC | 0.0 |
| lymphocyte act | | TNF alpha + IL-1beta | |
| CD8 lymphocyte act | 1.6 | Astrocytes rest | 0.0 |
| Secondary CD8 | 2.1 | Astrocytes TNF alpha + | 0.6 |
| lymphocyte rest | | IL-1beta | |
| Secondary CD8 | 1.2 | KU-812 (Basophil) rest | 3.4 |
| lymphocyte act | | | |
| CD4 lymphocyte none | 0.4 | KU-812 (Basophil) | 1.8 |
| | | PMA/ionomycin | |
| 2ry Th1/Th2/Tr1_anti- | 2.3 | CCD1106 (Keratinocytes) | 0.0 |
| CD95 CH11 | | none | |
| LAK cells rest | 0.9 | CCD1106 (Keratinocytes) | 0.0 |
| | | TNF alpha + IL-1beta | |
| LAK cells IL-2 | 0.8 | Liver cirrhosis | 0.7 |
| LAK cells IL-2 + IL-12 | 2.0 | NCI-H292 none | 2.2 |
| LAK cells IL-2 + IFN | 1.9 | NCI-H292 IL-4 | 4.3 |
| gamma | | | |
| LAK cells IL-2 + IL-18 | 0.0 | NCI-H292 IL-9 | 1.0 |
| LAK cells | 0.0 | NCI-H292 IL-13 | 1.8 |
| PMA/ionomycin | | | |
| NK Cells IL-2 rest | 2.0 | NCI-H292 IFN gamma | 1.3 |
| Two Way MLR 3 day | 3.4 | HPAEC none | 0.4 |
| Two Way MLR 5 day | 0.0 | HPAEC TNF alpha + IL- | 0.6 |
| | | 1beta | |
| Two Way MLR 7 day | 0.6 | Lung fibroblast none | 0.6 |
| PBMC rest | 0.5 | Lung fibroblast TNF alpha + | 0.0 |
| | | IL-1beta | |
| PBMC PWM | 0.0 | Lung fibroblast IL-4 | 1.1 |
| PBMC PHA-L | 0.0 | Lung fibroblast IL-9 | 0.0 |
| Ramos (B cell) none | 0.0 | Lung fibroblast IL-13 | 3.5 |
| Ramos (B cell) | 0.0 | Lung fibroblast IFN | 2.8 |
| ionomycin | | gamma | |
| B lymphocytes PWM | 0.0 | Dermal fibroblast | 2.2 |
| | | CCD1070 rest | |
| B lymphocytes CD40L | 2.4 | Dermal fibroblast | 7.3 |
| and IL-4 | | CCD1070 TNF alpha | |
| EOL-1 dbcAMP | 5.3 | Dermal fibroblast | 0.0 |
| | | CCD1070 IL-1beta | |
| EOL-1 dbcAMP | 1.2 | Dermal fibroblast IFN | 0.0 |
| PMA/ionomycin | | gamma | |
| Dendritic cells none | 2.5 | Dermal fibroblast IL-4 | 1.3 |
| Dendritic cells LPS | 0.9 | Dermal Fibroblasts rest | 0.5 |
| Dendritic cells anti- | 2.3 | Neutrophils TNFa + LPS | 0.0 |
| CD40 | | | |
| Monocytes rest | 0.0 | Neutrophils rest | 1.2 |
| Monocytes LPS | 0.0 | Colon | 3.1 |
| Macrophages rest | 1.8 | Lung | 6.7 |
| Macrophages LPS | 0.9 | Thymus | 32.5 |
| HUVEC none | 0.8 | Kidney | 100.0 |
| HUVEC starved | 0.5 | | |

CNS_neurodegeneration_v1.0 Summary: Ag2922 Expression of the NOV31 gene is low/undetectable in all samples on this panel (CTs>35). (Data not shown.)

Panel 1.3D Summary: Ag2922 Expression of the NOV31 gene is low/undetectable in all samples on this panel (CTs>35). (Data not shown.)

Panel 2D Summary: Ag2922 Results from one experiment with the NOV31 gene are not included. The amp plot indicates that there were experimental difficulties with this run.

Panel 4.1D Summary: Ag2922 Expression of the NOV31 gene is restricted to normal thymus and kidney (CTs=32-33). Thus, expression of this gene could be used as a marker for kidney and thymic tissue.

Panel 4D Summary: Ag2922 Results from one experiment with the NOV31 gene are not included. The amp plot indicates that there were experimental difficulties with this run.

NOV36a and NOV36b

Expression of gene NOV36a and variant NOV36b was assessed using the primer-probe sets Ag1136 and Ag2999, described in Tables AFA and AFB. Results of the RTQ-PCR runs are shown in Tables AFC, AFD, AFE and AFF.

TABLE AFA

Probe Name Ag1136

| | | | Start | |
|---|---|---|---|---|
| Primers | Sequences | Length | Position | SEQ ID NO: |
| Forward | 5′-tcatcaaagtgcaagacatcaa-3′ | 22 | 455 | 1105 |
| Probe | TET-5′-ttttccccttgggccctaccatg-3′- | 23 | 492 | 1106 |
| | TAMRA | | | |
| Reverse | 5′-gctactgttgccaacttcatct-3′ | 22 | 1560 | 1110 |

TABLE AEB

Probe Name Ag2999

| | | | Start | |
|---|---|---|---|---|
| Primers | Sequences | Length | Position | SEQ ID NO: |
| Forward | 5′-gccctacgatacttttgtgtgt-3′ | 22 | 1488 | 1108 |
| Probe | TET-5′-ctcctggccagctgattcaggtcat-3′- | 25 | 1520 | 1109 |
| | TAMRA | | | |
| Reverse | 5′-gctactgttgccaacttcatct-3′ | 22 | 1560 | 1110 |

TABLE AFC
CNS_neurodegeneration_v1.0

| | Rel. Exp. (%) Ag1136, | | Rel. Exp. (%) Ag1136, |
|---|---|---|---|
| Tissue Name | Run 206992276 | Tissue Name | Run 206992276 |
| AD 1 Hippo | 14.4 | Control (Path) 3 | 6.2 |
| | | Temporal Ctx | |
| AD 2 Hippo | 44.4 | Control (Path) 4 | 25.7 |
| | | Temporal Ctx | |
| AD 3 Hippo | 7.6 | AD 1 Occipital Ctx | 12.2 |
| AD 4 Hippo | 6.8 | AD 2 Occipital Ctx | 0.0 |
| | | (Missing) | |
| AD 5 hippo | 91.4 | AD 3 Occipital Ctx | 9.0 |
| AD 6 Hippo | 48.3 | AD 4 Occipital Ctx | 17.7 |
| Control 2 Hippo | 35.6 | AD 5 Occipital Ctx | 12.5 |
| Control 4 Hippo | 14.3 | AD 6 Occipital Ctx | 35.4 |
| Control (Path) 3 | 6.4 | Control 1 Occipital | 13.6 |
| Hippo | | Ctx | |
| AD 1 Temporal Ctx | 16.7 | Control 2 Occipital | 61.1 |
| | | Ctx | |
| AD 2 Temporal Ctx | 31.6 | Control 3 Occipital | 25.5 |
| | | Ctx | |
| AD 3 Temporal Ctx | 15.5 | Control 4 Occipital | 8.4 |
| | | Ctx | |
| AD 4 Temporal Ctx | 21.6 | Control (Path) 1 | 73.7 |
| | | Occipital Ctx | |
| AD 5 Inf Temporal | 100.0 | Control (Path) 2 | 9.2 |
| Ctx | | Occipital Ctx | |
| AD 5 SupTemporal | 41.8 | Control (Path) 3 | 4.6 |
| Ctx | | Occipital Ctx | |
| AD 6 Inf Temporal | 57.0 | Control (Path) 4 | 21.3 |
| Ctx | | Occipital Ctx | |
| AD 6 Sup Temporal | 37.4 | Control 1 Parietal | 10.6 |
| Ctx | | Ctx | |
| Control 1 Temporal | 16.5 | Control 2 Parietal | 46.0 |
| Ctx | | Ctx | |
| Control 2 Temporal | 45.7 | Control 3 Parietal | 18.7 |
| Ctx | | Ctx | |
| Control 3 Temporal | 20.4 | Control (Path) 1 | 62.9 |
| Ctx | | Parietal Ctx | |
| Control 4 Temporal | 12.6 | Control (Path) 2 | 17.9 |
| Ctx | | Parietal Ctx | |
| Control (Path) 1 | 51.1 | Control (Path) 3 | 10.1 |
| Temporal Ctx | | Parietal Ctx | |
| Control (Path) 2 | 31.6 | Control (Path) 4 | 39.5 |
| Temporal Ctx | | Parietal Ctx | |

TABLE AFD
Panel 1.3D

| | Rel. Exp. (%) Ag1136, | | Rel. Exp. (%) Ag1136, |
|---|---|---|---|
| Tissue Name | Run 165528214 | Tissue Name | Run 165528214 |
| Liver adenocarcinoma | 36.6 | Kidney (fetal) | 19.1 |
| Pancreas | 4.9 | Renal ca. 786-0 | 30.1 |
| Pancreatic ca. CAPAN 2 | 12.0 | Renal ca. A498 | 25.9 |
| Adrenal gland | 4.7 | Renal ca. RXF 393 | 18.6 |
| Thyroid | 4.5 | Renal ca. ACHN | 22.2 |
| Salivary gland | 0.6 | Renal ca. UO-31 | 22.4 |
| Pituitary gland | 10.0 | Renal ca. TK-10 | 31.4 |
| Brain (fetal) | 53.2 | Liver | 0.0 |
| Brain (whole) | 76.3 | Liver (fetal) | 0.9 |
| Brain (amygdala) | 43.5 | Liver ca. | 4.3 |
| | | (hepatoblast) HepG2 | |
| Brain (cerebellum) | 48.3 | Lung | 9.7 |
| Brain (hippocampus) | 42.6 | Lung (fetal) | 9.7 |
| Brain (substantia nigra) | 13.9 | Lung ca. (small cell) | 6.7 |
| | | LX-1 | |
| Brain (thalamus) | 37.6 | Lung ca. (small cell) | 23.3 |
| | | NCI-H69 | |
| Cerebral Cortex | 28.1 | Lung ca. (s.cell var.) | 49.7 |
| | | SHP-77 | |
| Spinal cord | 7.6 | Lung ca. (large | 13.9 |
| | | cell)NCI-H460 | |
| glio/astro U87-MG | 12.7 | Lung ca. (non-sm. | 8.4 |
| | | cell) A549 | |
| glio/astro U-118-MG | 85.9 | Lung ca. (non-s.cell) | 19.2 |
| | | NCI-H23 | |
| astrocytoma SW1783 | 33.9 | Lung ca. (non-s.cell) | 30.4 |
| | | HOP-62 | |
| neuro*; met SK-N-AS | 72.2 | Lung ca. (non-s.cl) | 32.5 |
| | | NCI-H522 | |
| astrocytoma SF-539 | 51.1 | Lung ca. (squam.) | 47.6 |
| | | SW 900 | |
| astrocytoma SNB-75 | 39.5 | Lung ca. (squam.) | 80.7 |
| | | NCI-H596 | |
| glioma SNB-19 | 17.8 | Mammary gland | 17.2 |
| glioma U251 | 100.0 | Breast ca.* (pl.ef) | 38.4 |
| | | MCF-7 | |
| glioma SF-295 | 13.5 | Breast ca.* (pl.ef) | 43.5 |
| | | MDA-MB-231 | |
| Heart (fetal) | 12.3 | Breast ca.* (pl.ef) | 13.8 |
| | | T47D | |
| Heart | 13.5 | Breast ca. BT-549 | 61.1 |
| Skeletal muscle (fetal) | 8.0 | Breast ca. MDA-N | 3.2 |
| Skeletal muscle | 0.0 | Ovary | 5.2 |
| Bone marrow | 1.6 | Ovarian ca. OVCAR-3 | 19.2 |
| Thymus | 26.4 | Ovarian ca. OVCAR-4 | 35.8 |
| Spleen | 15.1 | Ovarian ca. OVCAR-5 | 46.0 |
| Lymph node | 23.3 | Ovarian ca. OVCAR-8 | 54.7 |
| Colorectal | 6.3 | Ovarian ca. IGROV-1 | 10.6 |
| Stomach | 19.8 | Ovarian ca.* (ascites) | 24.8 |
| | | SK-OV-3 | |
| Small intestine | 40.1 | Uterus | 82.9 |
| Colon ca. SW480 | 16.0 | Placenta | 18.0 |
| Colon ca.* | 4.7 | Prostate | 5.0 |
| SW620(SW480 met) | | | |
| Colon ca. HT29 | 3.0 | Prostate ca.* (bone | 15.8 |
| | | met)PC-3 | |
| Colon ca. HCT-116 | 46.0 | Testis | 13.3 |
| Colon ca. CaCo-2 | 10.1 | Melanoma | 3.6 |
| | | Hs688(A).T | |
| Colon ca. | 25.7 | Melanoma* (met) | 6.2 |
| tissue(ODO3866) | | Hs688(B).T | |
| Colon ca. HCC-2998 | 52.1 | Melanoma UACC-62 | 8.8 |
| Gastric ca.* (liver met) | 26.2 | Melanoma M14 | 6.9 |
| NCI-N87 | | | |
| Bladder | 9.0 | Melanoma LOX | 5.6 |
| | | IMVI | |
| Trachea | 20.6 | Melanoma* (met) | 0.8 |
| | | SK-MEL-5 | |
| Kidney | 8.5 | Adipose | 9.9 |

TABLE AFE
Panel 2D

| | Rel. Exp. (%) | | Rel. Exp. (%) |
|---|---|---|---|
| | Ag1136, Run | | Ag1136, Run |
| Tissue Name | 162599391 | Tissue Name | 162599391 |
| Normal Colon | 36.1 | Kidney Margin | 10.9 |
| | | 8120608 | |
| CC Well to Mod Diff | 29.7 | Kidney Cancer | 9.7 |
| (ODO3866) | | 8120613 | |
| CC Margin (ODO3866) | 12.2 | Kidney Margin | 13.8 |
| | | 8120614 | |
| CC Gr.2 rectosigmoid | 16.3 | Kidney Cancer | 15.3 |
| (ODO3868) | | 9010320 | |
| CC Margin (ODO3868) | 7.0 | Kidney Margin | 29.1 |
| | | 9010321 | |
| CC Mod Diff (ODO3920) | 24.7 | Normal Uterus | 13.4 |
| CC Margin (ODO3920) | 10.2 | Uterus Cancer 064011 | 42.9 |
| CC Gr.2 ascend colon | 31.9 | Normal Thyroid | 12.8 |
| (ODO3921) | | | |
| CC Margin (ODO3921) | 10.4 | Thyroid Cancer | 20.4 |
| | | 064010 | |
| CC from Partial | 9.1 | Thyroid Cancer | 27.0 |
| Hepatectomy (ODO4309) | | A302152 | |
| Mets | | | |
| Liver Margin (ODO4309) | 2.3 | Thyroid Margin | 23.2 |
| | | A302153 | |
| Colon mets to lung | 22.1 | Normal Breast | 31.9 |
| (OD04451-01) | | | |
| Lung Margin (OD04451- | 13.2 | Breast Cancer | 37.9 |
| 02) | | (OD04566) | |
| Normal Prostate 6546-1 | 85.9 | Breast Cancer | 31.6 |
| | | (OD04590-01) | |
| Prostate Cancer | 34.9 | Breast Cancer Mets | 44.1 |
| (OD04410) | | (OD04590-03) | |
| Prostate Margin | 25.9 | Breast Cancer | 26.6 |
| (OD04410) | | Metastasis | |
| | | (OD04655-05) | |
| Prostate Cancer | 24.1 | Breast Cancer 064006 | 21.6 |
| (OD04720-01) | | | |
| Prostate Margin | 26.4 | Breast Cancer 1024 | 21.6 |
| (OD04720-02) | | | |
| Normal Lung 061010 | 40.3 | Breast Cancer | 38.7 |
| | | 9100266 | |
| Lung Met to Muscle | 100.0 | Breast Margin | 21.9 |
| (ODO4286) | | 9100265 | |
| Muscle Margin | 8.2 | Breast Cancer | 41.8 |
| (ODO4286) | | A209073 | |
| Lung Malignant Cancer | 28.3 | Breast Margin | 14.4 |
| (OD03126) | | A2090734 | |
| Lung Margin (OD03126) | 28.1 | Normal Liver | 2.2 |
| Lung Cancer (OD04404) | 51.4 | Liver Cancer 064003 | 4.2 |
| Lung Margin (OD04404) | 5.6 | Liver Cancer 1025 | 3.1 |
| Lung Cancer (OD04565) | 27.7 | Liver Cancer 1026 | 8.1 |
| Lung Margin (OD04565) | 6.2 | Liver Cancer 6004-T | 1.3 |
| Lung Cancer (OD04237- | 84.1 | Liver Tissue 6004-N | 3.6 |
| 01) | | | |
| Lung Margin (OD04237- | 10.6 | Liver Cancer 6005-T | 20.6 |
| 02) | | | |
| Ocular Mel Met to Liver | 4.5 | Liver Tissue 6005-N | 1.2 |
| (ODO4310) | | | |
| Liver Margin (ODO4310) | 3.5 | Normal Bladder | 30.1 |
| Melanoma Mets to Lung | 19.8 | Bladder Cancer 1023 | 16.3 |
| (OD04321) | | | |
| Lung Margin (OD04321) | 21.9 | Bladder Cancer | 22.2 |
| | | A302173 | |
| Normal Kidney | 29.7 | Bladder Cancer | 37.1 |
| | | (OD04718-01) | |
| Kidney Ca, Nuclear grade | 30.4 | Bladder Normal | 12.1 |
| 2 (OD04338) | | Adjacent (OD04718- | |
| | | 03) | |
| Kidney Margin | 16.2 | Normal Ovary | 25.2 |
| (OD04338) | | | |
| Kidney Ca Nuclear grade | 17.9 | Ovarian Cancer | 33.2 |
| 1/2 (OD04339) | | 064008 | |
| Kidney Margin | 18.2 | Ovarian Cancer | 77.9 |
| (OD04339) | | (OD04768-07) | |
| Kidney Ca, Clear cell | 21.6 | Ovary Margin | 11.9 |
| type (OD04340) | | (OD04768-08) | |
| Kidney Margin | 18.8 | Normal Stomach | 16.5 |
| (OD04340) | | | |
| Kidney Ca, Nuclear grade | 10.4 | Gastric Cancer | 8.4 |
| 3 (OD04348) | | 9060358 | |
| Kidney Margin | 22.1 | Stomach Margin | 5.8 |
| (OD04348) | | 9060359 | |
| Kidney Cancer | 8.4 | Gastric Cancer | 25.3 |
| (OD04622-01) | | 9060395 | |
| Kidney Margin | 5.6 | Stomach Margin | 14.7 |
| (OD04622-03) | | 9060394 | |
| Kidney Cancer | 42.0 | Gastric Cancer | 42.3 |
| (OD04450-01) | | 9060397 | |
| Kidney Margin | 13.6 | Stomach Margin 9060396 | 9.7 |
| (OD04450-03) | | | |
| Kidney Cancer 8120607 | 20.2 | Gastric Cancer | 21.9 |
| | | 064005 | |

TABLE AFF
Panel 4D

| | Rel. | Rel. | | Rel. | Rel. |
|---|---|---|---|---|---|
| | Exp. (%) | Exp. (%) | | Exp. (%) | Exp. (%) |
| | Ag1136, | Ag2999, | | Ag1136, | Ag2999, |
| | Run | Run | | Run | Run |
| Tissue Name | 164037277 | 165296355 | Tissue Name | 164037277 | 165296355 |
| Secondary Th1 act | 4.9 | 3.8 | HUVEC IL-1beta | 15.8 | 9.5 |
| Secondary Th2 act | 10.7 | 8.0 | HUVEC IFN | 47.0 | 61.1 |
| | | | gamma | | |
| Secondary Tr1 act | 12.2 | 10.2 | HUVEC TNF | 10.5 | 8.9 |
| | | | alpha + IFN | | |
| | | | gamma | | |
| Secondary Th1 rest | 4.9 | 5.8 | HUVEC TNF | 24.1 | 15.1 |
| | | | alpha + IL4 | | |
| Secondary Th2 rest | 7.5 | 3.0 | HUVEC IL-11 | 37.9 | 29.5 |
| Secondary Tr1 rest | 7.7 | 4.2 | Lung | 85.9 | 72.2 |
| | | | Microvascular EC | | |
| | | | none | | |
| Primary Th1 act | 5.7 | 7.3 | Lung | 37.1 | 33.9 |
| | | | Microvascular EC | | |
| | | | TNF alpha + IL- | | |
| | | | 1beta | | |
| Primary Th2 act | 9.1 | 6.7 | Microvascular | 85.9 | 24.7 |
| | | | Dermal EC none | | |
| Primary Tr1 act | 12.2 | 9.2 | Microsvasular | 26.6 | 14.8 |
| | | | Dermal EC | | |
| | | | TNF alpha + IL- | | |
| | | | 1beta | | |
| Primary Th1 rest | 21.0 | 20.7 | Bronchial | 21.8 | 15.8 |
| | | | epithelium | | |
| | | | TNF alpha + | | |
| | | | IL1beta | | |
| Primary Th2 rest | 13.6 | 8.5 | Small airway | 3.1 | 0.6 |
| | | | epithelium none | | |
| Primary Tr1 rest | 10.0 | 16.3 | Small airway | 25.3 | 15.0 |
| | | | epithelium | | |
| | | | TNF alpha + IL- | | |
| | | | 1beta | | |
| CD45RA CD4 | 10.7 | 8.2 | Coronery artery | 15.3 | 5.7 |
| lymphocyte act | | | SMC rest | | |
| CD45RO CD4 | 5.2 | 2.6 | Coronery artery | 3.1 | 4.6 |
| lymphocyte act | | | SMC TNF alpha + | | |
| | | | IL-1beta | | |
| CD8 lymphocyte | 4.5 | 3.2 | Astrocytes rest | 33.2 | 14.8 |
| act | | | | | |
| Secondary CD8 | 2.8 | 3.0 | Astrocytes | 18.7 | 15.4 |
| lymphocyte rest | | | TNF alpha + IL- | | |
| | | | 1beta | | |
| Secondary CD8 | 6.1 | 2.2 | KU-812 | 10.2 | 11.0 |
| lymphocyte act | | | (Basophil) rest | | |
| CD4 lymphocyte | 2.4 | 1.6 | KU-812 | 41.8 | 22.2 |
| none | | | (Basophil) | | |
| | | | PMA/ionomycin | | |
| 2ry | 7.5 | 10.0 | CCD1106 | 48.3 | 52.1 |
| Th1/Th2/Tr1_anti- | | | (Keratinocytes) | | |
| CD95 CH11 | | | none | | |
| LAK cells rest | 3.1 | 1.8 | CCD1106 | 47.3 | 46.7 |
| | | | (Keratinocytes) | | |
| | | | TNF alpha + IL- | | |
| | | | 1beta | | |
| LAK cells IL-2 | 4.7 | 3.0 | Liver cirrhosis | 5.7 | 1.8 |
| LAK cells IL-2 + IL- | 2.5 | 0.8 | Lupus kidney | 5.0 | 2.8 |
| 12 | | | | | |
| LAK cells IL- | 4.1 | 2.3 | NCI-H292 none | 22.8 | 11.4 |
| 2 + IFN gamma | | | | | |
| LAK cells IL-2 + | 3.1 | 1.0 | NCI-H292 IL-4 | 40.1 | 26.1 |
| IL-18 | | | | | |
| LAK cells | 6.3 | 4.3 | NCI-H292 IL-9 | 32.1 | 23.2 |
| PMA/ionomycin | | | | | |
| NK Cells IL-2 rest | 11.2 | 7.7 | NCI-H292 IL-13 | 20.9 | 24.0 |
| Two Way MLR 3 | 2.9 | 3.0 | NCI-H292 IFN | 20.4 | 16.6 |
| day | | | gamma | | |
| Two Way MLR 5 | 2.7 | 2.4 | HPAEC none | 67.8 | 66.4 |
| day | | | | | |
| Two Way MLR 7 | 2.9 | 0.8 | HPAEC TNF | 16.5 | 17.1 |
| day | | | alpha + IL-1beta | | |
| PBMC rest | 0.7 | 2.8 | Lung fibroblast | 19.8 | 15.8 |
| | | | none | | |
| PBMC PWM | 12.7 | 6.6 | Lung fibroblast | 5.0 | 6.7 |
| | | | TNF alpha + IL-1 | | |
| | | | beta | | |
| PBMC PHA-L | 6.7 | 3.2 | Lung fibroblast | 42.9 | 30.1 |
| | | | IL-4 | | |
| Ramos (B cell) | 0.8 | 0.0 | Lung fibroblast | 49.3 | 29.5 |
| none | | | IL-9 | | |
| Ramos (B cell) | 1.0 | 2.2 | Lung fibroblast | 24.7 | 21.0 |
| ionomycin | | | IL-13 | | |
| B lymphocytes | 9.7 | 4.3 | Lung fibroblast | 40.3 | 33.9 |
| PWM | | | IFN gamma | | |
| B lymphocytes | 5.2 | 5.0 | Dermal fibroblast | 75.3 | 52.5 |
| CD40L and IL-4 | | | CCD1070 rest | | |
| EOL-1 dbcAMP | 11.9 | 3.7 | Dermal fibroblast | 55.5 | 22.2 |
| | | | CCD1070 TNF | | |
| | | | alpha | | |
| EOL-1 dbcAMP | 17.8 | 7.7 | Dermal fibroblast | 18.6 | 18.9 |
| PMA/ionomycin | | | CCD1070 IL-1 | | |
| | | | beta | | |
| Dendritic cells | 0.0 | 0.0 | Dermal fibroblast | 12.3 | 16.0 |
| none | | | IFN gamma | | |
| Dendritic cells LPS | 0.4 | 0.0 | Dermal fibroblast | 16.6 | 13.2 |
| | | | IL-4 | | |
| Dendritic cells anti- | 0.0 | 0.5 | IBD Colitis 2 | 0.8 | 2.2 |
| CD40 | | | | | |
| Monocytes rest | 0.0 | 0.0 | IBD Crohn's | 1.2 | 1.0 |
| Monocytes LPS | 0.6 | 0.4 | Colon | 12.6 | 6.9 |
| Macrophages rest | 0.8 | 0.8 | Lung | 7.7 | 4.0 |
| Macrophages LPS | 0.0 | 0.4 | Thymus | 13.8 | 9.5 |
| HUVEC none | 45.4 | 54.0 | Kidney | 73.7 | 39.0 |
| HUVEC starved | 100.0 | 100.0 | | | |

CNS_neurodegeneration_v1.0 Summary: Ag1136 This panel does not show differential expression of the NOV36a gene in Alzheimer's disease. However, this expression profile confirms the presence of this gene in the brain. Please see Panel 1.3D for discussion of utility of this gene in the central nervous system.

Panel 1.3D Summary: Ag1136 Highest expression of the NOV36a gene, a cadherin 11 homolog, is seen in a glioma cell line U251 (CT=30.1). There is also low but significant significant expression in clusters of ovarian, lung, breast, kidney and colon cancer cell lines. Thus, expression of this gene could potentially be used as a diagnostic marker for these cancers. Furthermore, therapeutic inhibition of this gene product may be useful in the treatment of these cancers.

This gene also exhibits brain-preferential expression, indicating a role in CNS-specific processes. Recent research has shown that genetic deletion of cadherin-11 function acts to increase long term potentiation, a process thought to underlie learning and memory. Thus, drugs that target this gene prodcut may have utility as memory enhancing drugs. Such drugs would have utility in treatment of CNS disorders involving memory dysfunction, including Alzheimer's disease and normal aging. In behavioral tests, Cadherin 11 deletion mutant mice show reduced fear- or anxiety-related responses. Thus, inhibitory agents targeting this gene product may also have utility as sedatives or anxiolytic agents for the treatment of CNS disorders including anxiety.

Among tissues with metabolic function, this gene has low levels of expression in pancreas, adrenal, thyroid, pituitary, adult and fetal heart, and adipose. Thus, this cadherin-like gene product may be important in the pathogenesis, diagnosis, and/or treatment of metabolic and endocrine disease, including obesity and Types 1 and 2 diabetes. Decreased levels of cadherin have been associated with decreased insulin secretion, suggesting that increasing cadherin levels may be a potent therapeutic for Type 2 diabetes. In addition, this gene is expressed at higher levels in fetal (CT=34) vs adult skeletal muscle (CT=40) and may be useful for differentiation between the two sources of tissue. Ag2999 Results from one experiment with the CG56003-01 gene are not included. The amp plot indicates that there were experimental difficulties with this run.

References:

Manabe T, Togashi H, Uchida N, Suzuki S C, Hayakawa Y, Yamamoto M, Yoda H, Miyakawa T, Takeichi M, Chisaka O. Loss of cadherin-11 adhesion receptor enhances plastic changes in hippocampal synapses and modifies behavioral responses. Mol Cell Neurosci June 2000; 15(6):534-46

Cadherins organize symmetrical junctions between the pre- and postsynaptic membranes in central synapses. One of them, cadherin-11 (cad11), is expressed in the limbic system of the brain, most strongly in the hippocampus. Immunohistochemical studies of the hippocampus showed that cad11 proteins were densely distributed in its synaptic neuropil zones; in cultured hippocampal neurons, their distribution often overlapped with that of synaptophysin, and also occasionally with that of GluR1 at spines. To assess the role of cad11 in synaptic formation and/or function, we analyzed brains of cad11-deficient mice. In these mice, long-term potentiation (LTP) in the CA1 region of the hippocampus was, unexpectedly, enhanced; and the level of LTP saturation was increased. In behavioral tests, the mutant mice showed reduced fear- or anxiety-related responses. These results suggest that the cad11-mediated junctions may modulate synaptic efficacy, confining its dynamic changes to a limited range, or these junctions are required for normal development of synaptic organization in the hippocampus.

PMID: 10860580

Yamagata K, Nammo T, Moriwaki M, Ihara A, Iizuka K, Yang Q, Satoh T, Li M, Uenaka R, Okita K, Iwahashi H, Zhu Q, Cao Y, Imagawa A, Tochino Y, Hanafusa T, Miyagawa Ji J, Matsuzawa Y. Overexpression of Dominant-Negative Mutant Hepatocyte Nuclear Factor-1alpha in Pancreatic beta-Cells Causes Abnormal Islet Architecture With Decreased Expression of E-Cadherin, Reduced beta-cell Proliferation, and Diabetes. Diabetes. January 2002;51(1):114-23.

One subtype of maturity-onset diabetes of the young (MODY)-3 results from mutations in the gene encoding hepatocyte nuclear factor (HNF)-1alpha. We generated transgenic mice expressing a naturally occurring dominant-negative form of human HNF-1alpha (P291fsinsC) in pancreatic beta-cells. A progressive hyperglycemia with age was seen in these transgenic mice, and the mice developed diabetes with impaired glucose-stimulated insulin secretion. The pancreatic islets exhibited abnormal architecture with reduced expression of glucose transporter (GLUT2) and E-cadherin. Blockade of E-cadherin-mediated cell adhesion in pancreatic islets abolished the glucose-stimulated increases in intracellular Ca(2+) levels and insulin secretion, suggesting that loss of E-cadherin in beta-cells is associated with impaired insulin secretion. There was also a reduction in beta-cell number (50%), proliferation rate (15%), and pancreatic insulin content (45%) in 2-day-old transgenic mice and a further reduction in 4-week-old animals. Our findings suggest various roles for HNF-1alpha in normal glucose metabolism, including the regulation of glucose transport, beta-cell growth, and beta-cell-to-beta-cell communication.

PMID: 11756330

Panel 2D Summary: Ag1136 The NOV36a gene is a good target for ovarian, gastric, breast, lung, colon, uterine and kidney cancers because it is expressed at a higher level in these cancers than the adjacent normal tissue. Therefore, expression of this gene could potentially be used as a diagnostic marker for these cancers and therapeutic inhibition may be useful in treatment of these cancers.

Panel 4D Summary: Ag1136/Ag2999 Two experiments produce results that are in excellent agreement. The NOV36A transcript is expressed at low levels in hematopoietic cells and at higher levels in endothelial cells. IL-1 beta and TNFalpha treatment reduce transcript levels consistently in endothelium samples including HPAEC, HUVEC and lung microvascular EC. Fibroblasts also express this transcript and dermal fibroblasts down regulate expression in response to IL-1 beta, gamma interferon and IL-4. This transcript encodes a putative cadherin 11 like molecule. Cadherins are adhesion molecules that regulate normal homeostasis. Loss of cadherin 11 expression can reduce the expression of factors such as VEGF-D in fibroblasts (see reference 1). Therapies designed with the protein encoded by this transcript could be important in the regulating endothelium function including leukocyte extravasation, a major component of inflammation during asthma, IBD, and psoriasis. Therapeutics designed with the protein encoded by this transcript could also be important in the treatment of osteoarthritis and osteoporosis since this protein may be important in maintaining bone density (see reference 2).

References:

Orlandini M, Oliviero S In fibroblasts Vegf-D expression is induced by cell-cell contact mediated by cadherin-11. J Biol Chem Mar. 2, 2001;276(9):6576-81

Vascular endothelial growth factors (VEGFs) are a highly conserved family of growth factors all angiogenic in vivo with mitogenic and chemotactic activity on endothelial cells. VEGFs are expressed in fibroblasts either in hypoxia or in response to growth factors. Here we report that, differently from the other members of the family, Vegf-D is induced by cell-cell contact. By in situ hybridization we demonstrated that noninteracting fibroblasts express low levels of Vegf-D mRNA, whereas contacting cells express high levels of Vegf-D transcripts. By immunostaining we observed that the surface protein cadherin-11 is localized at the opposite sites of interacting cell surfaces. Ca(2+) deprivation from the culture medium determined the loss of cadherin-11 from the cell surfaces and down-regulation of Vegf-D mRNA. Moreover, a cadherin-11 antisense RNA construct inhibited Vegf-D expression in confluent BALB/c fibroblasts, whereas in NIH 3T3 cells, which express low levels of cadherin-11, Vegf-D induction could be obtained by overexpression of cadherin-11. This suggests that cell interaction mediated by cadherin-11 induces the expression of the angiogenic factor Vegf-D in fibroblasts.

PMID: 11108717

Kawaguchi J, Azuma Y, Hoshi K, Kii I, Takeshita S, Ohta T, Ozawa H, Takeichi M, Chisaka O, Kudo A. Targeted disruption of cadherin-11 leads to a reduction in bone density in calvaria and long bone metaphyses. J Bone Miner Res July 2001;16(7):1265-71

The migration and adhesion of osteoblasts requires several classical cadherins. Cadherin-11, one of the classical cadherins, was expressed in mouse osteoblasts in skull bone and femur, revealed by immunohistochemistry. To elucidate the function of cadherin-11 in osteoblastogenesis, cadherin-11 null mutant mice were investigated. Although apparently normal at birth, Alizarin red staining of null mutant mice showed a reduced calcified area at the frontal suture that caused a round-shaped calvaria with increasing animal age to 3 months.

Consequently, there was a reduction in bone density at the femoral metaphyses and the diploe of calvaria in null mutant mice. In the in vitro culture of newborn calvarial cells, the calcified area of mutant cells was smaller than those derived from wild-type littermates. These results show that absence of cadherin-11 leads to reduced bone density in some parts of skeletons including calvaria and long bone metaphyses, and thus suggest that cadherin-11 plays roles in the regulation of osteoblast differentiation and in the mineralization of the osteoid matrix.

PMID: 11450702

NOV37

Expression of gene NOV37 was assessed using the primer-probe sets Ag047, Ag2679, Ag2728, Ag332, Ag47b, Ag712, Ag2732 and Ag2975, described in Tables AGA, AGB, AGC, AGD, AGE, AGF, AGG and AGH. Results of the RTQ-PCR runs are shown in Tables AGI, AGJ, AGK, AGL, AGM, AGN, AGO, AGP, AGQ and AGR.

TABLE AGA

Probe Name Ag047

| | | | Start | |
|---|---|---|---|---|
| Primers | Sequences | Length | Position | SEQ ID NO: |
| Forward | 5′-ccaatgacctggccacca-3′ | 18 | 1064 | 1111 |
| Probe | TET-5′-ccagagtccgttcagcttcaggacagc- | 27 | 1084 | 1112 |
| | 3′-TAMRA | | | |
| Reverse | 5′-gtggcacgttgctgtttagc-3′ | 20 | 1116 | 1113 |

TABLE AGB

Probe Name Ag2679

| | | | Start | |
|---|---|---|---|---|
| Primers | Sequences | Length | Position | SEQ ID NO: |
| Forward | 5′-ttgacctcaggaacggtttac-3′ | 21 | 1213 | 1114 |
| Probe | TET-5′-ctgctgcccaggaatactttctccag-3′- | 26 | 1249 | 1115 |
| | TAMRA | | | |
| Reverse | 5′-agtatttggagggcttcttcag-3′ | 22 | 1288 | 1116 |

TABLE AGC

Probe Name Ag2728

| | | | Start | |
|---|---|---|---|---|
| Primers | Sequences | Length | Position | SEQ ID NO: |
| Forward | 5′-ggagcttggtctcatgaccta-3′ | 21 | 5178 | 1117 |
| Probe | TET-5′-actgggctcctggccaccaagag-3′- | 23 | 5209 | 1118 |
| | TAMRA | | | |
| Reverse | 5′-agtcgtccatcctgtttcatc-3′ | 21 | 5233 | 1119 |

TABLE AGD

Probe Name Ag332

| | | | Start | |
|---|---|---|---|---|
| Primers | Sequences | Length | Position | SEQ ID NO: |
| Forward | 5′-gctgccctgacttgtgcaa-3′ | 19 | 2594 | 1120 |
| Probe | TET-5′-tctgacccagtgtgcatctcccgtt-3′- | 25 | 2617 | 1121 |
| | TAMRA | | | |
| Reverse | 5′-ccggtctggcagacacact-3′ | 19 | 2651 | 1122 |

TABLE AGE

Probe Name Ag47b

| | | | Start | |
|---|---|---|---|---|
| Primers | Sequences | Length | Position | SEQ ID NO: |
| Forward | 5′-gaacgccggagcatacaga-3′ | 19 | 1774 | 1123 |
| Probe | TET-5′-ccaggtactgcacaaacacggcttcat- | 27 | 1805 | 1124 |
| | 3′-TAMRA | | | |
| Reverse | 5′-gatgccacaggcccaca-3′ | 17 | 1833 | 1125 |

TABLE AGF

Probe Name Ag712

| | | | Start | |
|---|---|---|---|---|
| Primers | Sequences | Length | Position | SEQ ID NO: |
| Forward | 5′-tgcaagggtacgagggatat-3′ | 20 | 8369 | 1126 |
| Probe | TET-5′-cttcccgtggagcaatacccagag-3′ | 24 | 8395 | 1127 |
| | TAMRA | | | |
| Reverse | 5′-tggatgttgctgctactgtct-3′ | 21 | 8424 | 1128 |

TABLE AGG

Probe Name Ag2732

| | | | Start | |
|---|---|---|---|---|
| Primers | Sequences | Length | Position | SEQ ID NO: |
| Forward | 5′-ggagcttggtctcatgaccta-3′ | 21 | 5178 | 1129 |
| Probe | TET-5′-actgggctcctggccaccaagag-3′- | 23 | 5209 | 1130 |
| | TAMRA | | | |
| Reverse | 5′-agtcgtccatcctgtttcatc-3′ | 21 | 5233 | 1131 |

TABLE AGH

Probe Name Ag2975

| | | | Start | |
|---|---|---|---|---|
| Primers | Sequences | Length | Position | SEQ ID NO: |
| Forward | 5′-ggagcttggtctcatgaccta-3′ | 21 | 5178 | 1132 |
| Probe | TET-5′-actgggctcctggccaccaagag-3′- | 23 | 5209 | 1133 |
| | TAMRA | | | |
| Reverse | 5′-agtcgtccatcctgtttcatc-3′ | 21 | 5233 | 1134 |

TABLE AGI
CNS_neurodegeneration_v1.0

| | Rel. | Rel. | Rel. | | Rel. | Rel. | Rel. |
|---|---|---|---|---|---|---|---|
| | Exp. (%) | Exp. (%) | Exp. (%) | | Exp. (%) | Exp. (%) | Exp. (%) |
| | Ag047, | Ag2679, | Ag2728, | | Ag047, | Ag2679, | Ag2728, |
| Tissue | Run | Run | Run | Tissue | Run | Run | Run |
| Name | 206228021 | 209751329 | 206483376 | Name | 206228021 | 209751329 | 206483376 |
| AD 1 | 10.3 | 6.6 | 8.4 | Control | 4.1 | 3.2 | 2.3 |
| Hippo | | | | (Path) 3 | | | |
| | | | | Temporal | | | |
| | | | | Ctx | | | |
| AD 2 | 18.0 | 20.9 | 17.3 | Control | 13.8 | 33.9 | 29.7 |
| Hippo | | | | (Path) 4 | | | |
| | | | | Temporal | | | |
| | | | | Ctx | | | |
| AD 3 | 6.6 | 4.6 | 6.4 | AD 1 | 18.8 | 16.8 | 17.1 |
| Hippo | | | | Occipital | | | |
| | | | | Ctx | | | |
| AD 4 | 5.6 | 4.4 | 7.7 | AD 2 | 0.0 | 0.0 | 0.0 |
| Hippo | | | | Occipital | | | |
| | | | | Ctx | | | |
| | | | | (Missing) | | | |
| AD 5 | 100.0 | 100.0 | 95.9 | AD 3 | 5.7 | 3.4 | 3.1 |
| Hippo | | | | Occipital | | | |
| | | | | Ctx | | | |
| AD 6 | 37.6 | 33.0 | 31.4 | AD 4 | 12.9 | 15.1 | 19.5 |
| Hippo | | | | Occipital | | | |
| | | | | Ctx | | | |
| Control 2 | 22.1 | 17.3 | 43.2 | AD 5 | 25.2 | 33.4 | 51.4 |
| Hippo | | | | Occipital | | | |
| | | | | Ctx | | | |
| Control 4 | 7.7 | 6.4 | 5.3 | AD 6 | 3.2 | 18.9 | 19.8 |
| Hippo | | | | Occipital | | | |
| | | | | Ctx | | | |
| Control | 1.5 | 3.4 | 3.7 | Control 1 | 3.4 | 3.5 | 4.4 |
| (Path) 3 | | | | Occipital | | | |
| Hippo | | | | Ctx | | | |
| AD 1 | 8.1 | 6.7 | 8.6 | Control 2 | 83.5 | 82.9 | 100.0 |
| Temporal | | | | Occipital | | | |
| Ctx | | | | Ctx | | | |
| AD 2 | 21.2 | 40.3 | 21.2 | Control 3 | 25.3 | 20.2 | 17.3 |
| Temporal | | | | Occipital | | | |
| Ctx | | | | Ctx | | | |
| AD 3 | 3.3 | 4.2 | 4.2 | Control 4 | 5.4 | 4.2 | 4.4 |
| Temporal | | | | Occipital | | | |
| Ctx | | | | Ctx | | | |
| AD 4 | 18.7 | 13.1 | 20.0 | Control | 66.4 | 74.7 | 75.8 |
| Temporal | | | | (Path) 1 | | | |
| Ctx | | | | Occipital | | | |
| | | | | Ctx | | | |
| AD 5 Inf | 27.0 | 57.4 | 42.9 | Control | 15.2 | 11.3 | 13.0 |
| Temporal | | | | (Path) 2 | | | |
| Ctx | | | | Occipital | | | |
| | | | | Ctx | | | |
| AD 5 Sup | 14.6 | 27.0 | 20.7 | Control | 3.0 | 2.4 | 1.5 |
| Temporal | | | | (Path) 3 | | | |
| Ctx | | | | Occipital | | | |
| | | | | Ctx | | | |
| AD 6 Inf | 25.2 | 16.8 | 19.1 | Control | 38.7 | 29.3 | 19.6 |
| Temporal | | | | (Path) 4 | | | |
| Ctx | | | | Occipital | | | |
| | | | | Ctx | | | |
| AD 6 Sup | 26.1 | 18.9 | 17.2 | Control 1 | 4.4 | 3.7 | 4.1 |
| Temporal | | | | Parietal | | | |
| Ctx | | | | Ctx | | | |
| Control 1 | 3.9 | 3.3 | 3.8 | Control 2 | 31.4 | 19.8 | 18.2 |
| Temporal | | | | Parietal | | | |
| Ctx | | | | Ctx | | | |
| Control 2 | 21.3 | 27.4 | 33.4 | Control 3 | 7.9 | 10.7 | 9.6 |
| Temporal | | | | Parietal | | | |
| Ctx | | | | Ctx | | | |
| Control 3 | 13.7 | 10.0 | 10.4 | Control | 46.3 | 56.3 | 58.6 |
| Temporal | | | | (Path) 1 | | | |
| Ctx | | | | Parietal | | | |
| | | | | Ctx | | | |
| Control 3 | 8.1 | 7.9 | 8.7 | Control | 13.2 | 16.6 | 18.4 |
| Temporal | | | | (Path) 2 | | | |
| Ctx | | | | Parietal | | | |
| | | | | Ctx | | | |
| Control | 34.4 | 42.3 | 51.4 | Control | 2.5 | 2.4 | 1.2 |
| (Path) 1 | | | | (Path) 3 | | | |
| Temporal | | | | Parietal | | | |
| Ctx | | | | Ctx | | | |
| Control | 27.2 | 29.9 | 24.5 | Control | 35.1 | 41.2 | 35.6 |
| (Path) 2 | | | | (Path) 4 | | | |
| Temporal | | | | Parietal | | | |
| Ctx | | | | Ctx | | | |

TABLE AGJ
Panel 1

| | Rel. Exp. (%) | Rel. Exp. (%) | Rel. Exp. (%) | Rel. Exp. (%) | Rel. Exp. (%) |
|---|---|---|---|---|---|
| | Ag047, Run | Ag047, Run | Ag332, Run | Ag332, Run | Ag47b, Run |
| Tissue Name | 87354354 | 87354779 | 97803603 | 98747043 | 88164379 |
| Endothelial cells | 0.0 | 0.0 | 0.0 | 0.0 | 0.0 |
| Endothelial cells | 0.0 | 0.0 | 0.0 | 0.0 | 0.0 |
| (treated) | | | | | |
| Pancreas | 0.0 | 0.3 | 0.0 | 0.0 | 0.0 |
| Pancreatic ca. | 0.0 | 0.0 | 0.0 | 0.0 | 0.0 |
| CAPAN 2 | | | | | |
| Adrenal gland | 0.0 | 1.3 | 0.2 | 0.0 | 0.0 |
| Thyroid | 0.0 | 0.4 | 100.0 | 0.1 | 0.0 |
| Salivary gland | 0.0 | 0.2 | 0.0 | 0.0 | 0.0 |
| Pituitary gland | 0.0 | 0.1 | 2.1 | 0.0 | 0.0 |
| Brain (fetal) | 0.0 | 15.0 | 3.4 | 11.0 | 21.8 |
| Brain (whole) | 95.9 | 67.8 | 3.2 | 4.5 | 32.5 |
| Brain (amygdala) | 0.0 | 8.8 | 3.8 | 8.7 | 12.2 |
| Brain | 0.0 | 22.2 | 1.4 | 0.1 | 14.3 |
| (cerebellum) | | | | | |
| Brain | 0.0 | 24.7 | 3.4 | 8.7 | 15.6 |
| (hippocampus) | | | | | |
| Brain (substantia | 3.4 | 3.6 | 1.3 | 2.3 | 3.8 |
| nigra) | | | | | |
| Brain (thalamus) | 3.5 | 4.7 | 3.3 | 5.0 | 9.1 |
| Brain | 0.0 | 0.7 | 0.2 | 0.0 | 0.0 |
| (hypothalamus) | | | | | |
| Spinal cord | 0.7 | 1.5 | 0.9 | 1.4 | 1.1 |
| glio/astro U87- | 0.6 | 2.6 | 1.6 | 1.7 | 3.0 |
| MG | | | | | |
| glio/astro U-118- | 0.0 | 0.6 | 0.2 | 1.1 | 0.1 |
| MG | | | | | |
| astrocytoma | 33.7 | 29.5 | 9.0 | 42.9 | 36.1 |
| SW1783 | | | | | |
| neuro*; met SK- | 0.0 | 0.0 | 0.0 | 0.0 | 0.0 |
| N-AS | | | | | |
| astrocytoma SF- | 31.0 | 36.9 | 10.3 | 68.3 | 48.3 |
| 539 | | | | | |
| astrocytoma SNB- | 33.7 | 32.8 | 7.1 | 23.0 | 50.3 |
| 75 | | | | | |
| glioma SNB-19 | 100.0 | 100.0 | 30.1 | 100.0 | 100.0 |
| glioma U251 | 49.0 | 44.1 | 16.5 | 57.8 | 41.5 |
| glioma SF-295 | 6.0 | 8.1 | 2.1 | 19.8 | 8.7 |
| Heart | 61.1 | 26.8 | 36.6 | 70.7 | 39.8 |
| Skeletal muscle | 0.0 | 0.1 | 0.2 | 0.0 | 0.0 |
| Bone marrow | 0.0 | 0.1 | 0.0 | 0.0 | 0.0 |
| Thymus | 18.7 | 18.4 | 1.1 | 2.7 | 17.2 |
| Spleen | 0.0 | 0.0 | 0.0 | 0.0 | 0.0 |
| Lymph node | 0.0 | 0.2 | 0.0 | 0.0 | 0.0 |
| Colon (ascending) | 0.5 | 0.7 | 0.7 | 0.9 | 4.5 |
| Stomach | 0.1 | 1.1 | 0.2 | 0.2 | 0.2 |
| Small intestine | 0.0 | 0.1 | 0.0 | 0.0 | 0.0 |
| Colon ca. SW480 | 0.6 | 1.0 | 1.6 | 7.0 | 1.4 |
| Colon ca.* | 0.0 | 0.0 | 0.0 | 0.0 | 0.0 |
| SW620 (SW480 | | | | | |
| met) | | | | | |
| Colon ca. HT29 | 0.0 | 0.0 | 0.0 | 0.0 | 0.0 |
| Colon ca. HCT- | 0.0 | 0.0 | 0.0 | 0.0 | 0.0 |
| 116 | | | | | |
| Colon ca. CaCo-2 | 0.0 | 0.1 | 0.0 | 0.0 | 0.0 |
| Colon ca. HCT-15 | 0.0 | 0.1 | 0.2 | 0.0 | 0.0 |
| Colon ca. HCC- | 0.0 | 0.0 | 0.0 | 0.0 | 0.0 |
| 2998 | | | | | |
| Gastric ca.* (liver | 0.0 | 0.4 | 0.2 | 0.0 | 0.0 |
| met) NCI-N87 | | | | | |
| Bladder | 0.3 | 1.0 | 0.2 | 0.0 | 0.1 |
| Trachea | 0.0 | 0.4 | 0.2 | 0.0 | 0.0 |
| Kidney | 0.2 | 0.9 | 0.7 | 1.2 | 1.0 |
| Kidney (fetal) | 1.3 | 3.9 | 0.8 | 3.2 | 2.7 |
| Renal ca. 786-0 | 10.6 | 11.7 | 3.4 | 7.9 | 12.2 |
| Renal ca. A498 | 0.0 | 0.1 | 0.0 | 0.0 | 0.0 |
| Renal ca. RXF | 17.9 | 14.0 | 4.1 | 16.6 | 15.2 |
| 393 | | | | | |
| Renal ca. ACHN | 0.0 | 0.0 | 0.0 | 0.0 | 0.0 |
| Renal ca. UO-31 | 0.0 | 0.2 | 0.1 | 0.0 | 0.2 |
| Renal ca. TK-10 | 0.0 | 0.0 | 0.0 | 0.0 | 0.0 |
| Liver | 0.0 | 3.2 | 0.8 | 0.9 | 1.9 |
| Liver (fetal) | 0.0 | 0.1 | 0.0 | 0.0 | 0.0 |
| Liver ca. | 0.0 | 0.0 | 0.0 | 0.0 | 0.0 |
| (hepatoblast) | | | | | |
| HepG2 | | | | | |
| Lung | 0.0 | 0.2 | 0.0 | 0.0 | 0.0 |
| Lung (fetal) | 0.0 | 0.5 | 0.0 | 0.0 | 0.0 |
| Lung ca. (small | 0.0 | 0.0 | 0.0 | 0.0 | 0.0 |
| cell) LX-1 | | | | | |
| Lung ca. (small | 2.0 | 3.3 | 1.4 | 5.4 | 2.9 |
| cell) NCI-H69 | | | | | |
| Lung ca. (s.cell | 0.0 | 0.0 | 0.0 | 0.0 | 0.0 |
| var.) SHP-77 | | | | | |
| Lung ca. (large | 0.0 | 0.0 | 1.3 | 0.7 | 0.3 |
| cell)NCI-H460 | | | | | |
| Lung ca. (non-sm. | 0.0 | 0.0 | 0.1 | 0.0 | 0.0 |
| cell) A549 | | | | | |
| Lung ca. (non- | 0.0 | 0.0 | 0.0 | 0.0 | 0.0 |
| s.cell) NCI-H23 | | | | | |
| Lung ca. (non- | 5.2 | 4.7 | 3.9 | 11.3 | 6.7 |
| s.cell) HOP-62 | | | | | |
| Lung ca. (non- | 1.7 | 3.1 | 1.5 | 2.8 | 4.8 |
| s.cl) NCI-H522 | | | | | |
| Lung ca. (squam.) | 0.0 | 0.0 | 0.0 | 0.0 | 0.0 |
| SW 900 | | | | | |
| Lung ca. (squam.) | 1.3 | 2.5 | 2.0 | 4.2 | 3.0 |
| NCI-H596 | | | | | |
| Mammary gland | 11.9 | 9.1 | 4.6 | 7.0 | 10.6 |
| Breast ca.* (pl.ef) | 0.0 | 0.0 | 0.0 | 0.0 | 0.0 |
| MCF-7 | | | | | |
| Breast ca.* (pl.ef) | 0.0 | 0.1 | 0.0 | 0.0 | 0.0 |
| MDA-MB-231 | | | | | |
| Breast ca.* (pl. ef) | 11.8 | 7.5 | 7.9 | 31.6 | 31.4 |
| T47D | | | | | |
| Breast ca. BT-549 | 0.0 | 0.0 | 4.5 | 13.8 | 28.3 |
| Breast ca. MDA-N | 0.0 | 0.1 | 0.0 | 0.0 | 0.0 |
| Ovary | 0.1 | 0.6 | 0.4 | 0.1 | 0.2 |
| Ovarian ca. | 0.0 | 0.0 | 0.0 | 0.0 | 0.0 |
| OVCAR-3 | | | | | |
| Ovarian ca. | 0.0 | 0.1 | 1.3 | 4.4 | 0.0 |
| OVCAR-4 | | | | | |
| Ovarian ca. | 73.2 | 38.2 | 14.4 | 57.0 | 36.1 |
| OVCAR-5 | | | | | |
| Ovarian ca. | 0.0 | 0.7 | 1.0 | 3.4 | 0.2 |
| OVCAR-8 | | | | | |
| Ovarian ca. | 0.0 | 0.0 | 0.8 | 2.6 | 0.0 |
| IGROV-1 | | | | | |
| Ovarian ca. | 0.0 | 0.0 | 0.0 | 0.0 | 0.0 |
| (ascites) SK-OV-3 | | | | | |
| Uterus | 0.4 | 1.2 | 0.6 | 1.7 | 2.9 |
| Placenta | 0.0 | 0.1 | 0.0 | 0.0 | 0.0 |
| Prostate | 0.9 | 1.9 | 0.7 | 1.9 | 1.7 |
| Prostate ca.* | 0.0 | 0.0 | 0.1 | 0.0 | 0.0 |
| (bone met) PC-3 | | | | | |
| Testis | 25.7 | 22.2 | 0.7 | 0.7 | 26.4 |
| Melanoma | 23.8 | 21.6 | 5.0 | 20.0 | 28.9 |
| Hs688(A).T | | | | | |
| Melanoma* (met) | 4.6 | 6.5 | 3.4 | 9.6 | 9.2 |
| Hs688(B).T | | | | | |
| Melanoma | 0.0 | 0.0 | 0.0 | 0.0 | 0.0 |
| UACC-62 | | | | | |
| Melanoma M14 | 0.0 | 0.1 | 0.0 | 0.0 | 0.0 |
| Melanoma LOX | 3.7 | 3.4 | 0.6 | 0.2 | 2.7 |
| IMVI | | | | | |
| Melanoma* (met) | 0.0 | 0.0 | 0.0 | 0.0 | 0.0 |
| SK-MEL-5 | | | | | |
| Melanoma SK- | 0.0 | 0.0 | 3.6 | 17.4 | 0.0 |
| MEL-28 | | | | | |

TABLE AGK
Panel 1.1

| | Rel. Exp. (%) Ag047, | | Rel. Exp. (%) Ag047, |
|---|---|---|---|
| Tissue Name | Run 109663520 | Tissue Name | Run 109663520 |
| Adrenal gland | 0.5 | Renal ca. UO-31 | 0.1 |
| Bladder | 1.2 | Renal ca. RXF 393 | 8.7 |
| Brain (amygdala) | 2.8 | Liver | 1.3 |
| Brain (cerebellum) | 4.1 | Liver (fetal) | 0.0 |
| Brain (hippocampus) | 9.6 | Liver ca. | 0.0 |
| | | (hepatoblast) HepG2 | |
| Brain (substantia | 15.0 | Lung | 0.1 |
| nigra) | | | |
| Brain (thalamus) | 4.7 | Lung (fetal) | 0.4 |
| Cerebral Cortex | 48.3 | Lung ca. (non-s.cell) | 26.6 |
| | | HOP-62 | |
| Brain (fetal) | 21.2 | Lung ca. (large | 1.2 |
| | | cell)NCI-H460 | |
| Brain (whole) | 9.0 | Lung ca. (non-s.cell) | 0.0 |
| | | NCI-H23 | |
| glio/astro U-118-MG | 0.7 | Lung ca. (non-s.cl) | 5.8 |
| | | NCI-H522 | |
| astrocytoma SF-539 | 38.4 | Lung ca. (non-sm. | 0.0 |
| | | cell) A549 | |
| astrocytoma SNB-75 | 16.4 | Lung ca. (s.cell var.) | 0.0 |
| | | SHP-77 | |
| astrocytoma SW1783 | 19.1 | Lung ca. (small cell) | 0.0 |
| | | LX-1 | |
| glioma U251 | 55.5 | Lung ca. (small cell) | 5.3 |
| | | NCI-H69 | |
| glioma SF-295 | 8.7 | Lung ca. (squam.) | 0.0 |
| | | SW 900 | |
| glioma SNB-19 | 100.0 | Lung ca. (squam.) | 5.0 |
| | | NCI-H596 | |
| glio/astro U87-MG | 3.3 | Lymph node | 0.3 |
| neuro*; met SK-N-AS | 0.0 | Spleen | 0.0 |
| Mammary gland | 2.0 | Thymus | 0.6 |
| Breast ca. BT-549 | 6.4 | Ovary | 0.6 |
| Breast ca. MDA-N | 0.0 | Ovarian ca. IGROV-1 | 0.0 |
| Breast ca.* (pl.ef) | 5.7 | Ovarian ca. OVCAR-3 | 0.0 |
| T47D | | | |
| Breast ca.* (pl.ef) | 0.0 | Ovarian ca. OVCAR-4 | 0.1 |
| MCF-7 | | | |
| Breast ca.* (pl.ef) | 0.0 | Ovarian ca. OVCAR-5 | 49.0 |
| MDA-MB-231 | | | |
| Small intestine | 0.0 | Ovarian ca. OVCAR-8 | 0.5 |
| Colorectal | 0.1 | Ovarian ca.* (ascites) | 0.1 |
| | | SK-OV-3 | |
| Colon ca. HT29 | 0.0 | Pancreas | 0.6 |
| Colon ca. CaCo-2 | 0.0 | Pancreatic ca. | 0.0 |
| | | CAPAN 2 | |
| Colon ca. HCT-15 | 0.1 | Pituitary gland | 0.5 |
| Colon ca. HCT-116 | 0.0 | Placenta | 0.0 |
| Colon ca. HCC-2998 | 0.0 | Prostate | 0.8 |
| Colon ca. SW480 | 1.2 | Prostate ca.* (bone | 0.1 |
| | | met) PC-3 | |
| Colon ca.* SW620 | 0.0 | Salivary gland | 0.2 |
| (SW480 met) | | | |
| Stomach | 0.2 | Trachea | 0.5 |
| Gastric ca. (liver met) | 0.1 | Spinal cord | 1.4 |
| NCI-N87 | | | |
| Heart | 80.7 | Testis | 0.5 |
| Skeletal muscle (Fetal) | 1.6 | Thyroid | 0.3 |
| Skeletal muscle | 0.4 | Uterus | 0.0 |
| Endothelial cells | 0.0 | Melanoma M14 | 0.0 |
| Heart (Fetal) | 14.2 | Melanoma LOX | 0.7 |
| | | IMVI | |
| Kidney | 2.4 | Melanoma UACC-62 | 0.0 |
| Kidney (fetal) | 1.5 | Melanoma SK-MEL- | 0.0 |
| | | 28 | |
| Renal ca. 786-0 | 3.6 | Melanoma* (met) | 0.0 |
| | | SK-MEL-5 | |
| Renal ca. A498 | 0.0 | Melanoma | 16.5 |
| | | Hs688(A).T | |
| Renal ca. ACHN | 0.0 | Melanoma* (met) | 5.6 |
| | | Hs688(B).T | |
| Renal ca. TK-10 | 0.0 | | |

TABLE AGL
Panel 1.2

| | Rel. Exp. (%) | Rel. Exp. (%) | | Rel. Exp. (%) | Rel. Exp. (%) |
|---|---|---|---|---|---|
| | Ag712, Run | Ag712, Run | | Ag712, Run | Ag712, Run |
| Tissue Name | 114986148 | 119452123 | Tissue Name | 114986148 | 119452123 |
| Endothelial cells | 0.0 | 0.0 | Renal ca. 786-0 | 4.0 | 3.0 |
| Heart (Fetal) | 4.0 | 2.1 | Renal ca. A498 | 0.1 | 0.1 |
| Pancreas | 2.1 | 1.1 | Renal ca. RXF | 4.9 | 3.5 |
| | | | 393 | | |
| Pancreatic ca. | 0.0 | 0.0 | Renal ca. | 0.6 | 0.3 |
| CAPAN 2 | | | ACHN | | |
| Adrenal Gland | 2.8 | 2.2 | Renal ca. UO- | 0.3 | 0.1 |
| | | | 31 | | |
| Thyroid | 2.1 | 1.5 | Renal ca. TK- | 0.2 | 0.1 |
| | | | 10 | | |
| Salivary gland | 1.9 | 1.1 | Liver | 4.6 | 2.9 |
| Pituitary gland | 4.6 | 3.7 | Liver (fetal) | 0.4 | 0.3 |
| Brain (fetal) | 22.2 | 29.3 | Liver ca. | 0.0 | 0.0 |
| | | | (hepatoblast) | | |
| | | | HepG2 | | |
| Brain (whole) | 33.2 | 26.1 | Lung | 0.8 | 0.6 |
| Brain | 18.6 | 12.9 | Lung (fetal) | 1.3 | 1.2 |
| (amygdala) | | | | | |
| Brain | 2.3 | 1.8 | Lung ca. (small | 0.0 | 0.0 |
| (cerebellum) | | | cell) LX-1 | | |
| Brain | 27.5 | 18.0 | Lung ca. (small | 4.4 | 3.7 |
| (hippocampus) | | | cell) NCI-H69 | | |
| Brain (thalamus) | 10.4 | 7.7 | Lung ca. (s.cell | 0.1 | 0.0 |
| | | | var.) SHP-77 | | |
| Cerebral Cortex | 25.3 | 22.2 | Lung ca. (large | 1.0 | 0.6 |
| | | | cell) NCI-H460 | | |
| Spinal cord | 3.8 | 3.2 | Lung ca. (non- | 0.3 | 0.1 |
| | | | sm. cell) A549 | | |
| glio/astro U87- | 2.5 | 2.1 | Lung ca. (non- | 0.1 | 0.1 |
| MG | | | s.cell) NCI- | | |
| | | | H23 | | |
| glio/astro U-118- | 0.8 | 0.4 | Lung ca. (non- | 7.2 | 5.8 |
| MG | | | s.cell) HOP-62 | | |
| astrocytoma | 19.9 | 9.2 | Lung ca. (non- | 5.9 | 4.0 |
| SW1783 | | | s.cl) NCI-H522 | | |
| neuro*; met SK- | 0.1 | 0.0 | Lung ca. | 0.0 | 0.0 |
| N-AS | | | (squam.) SW | | |
| | | | 900 | | |
| astrocytoma SF- | 36.1 | 19.8 | Lung ca. | 4.6 | 3.7 |
| 539 | | | (squam.) NCI- | | |
| | | | H596 | | |
| astrocytoma | 10.7 | 3.6 | Mammary | 7.6 | 4.5 |
| SNB-75 | | | gland | | |
| glioma SNB-19 | 71.2 | 51.4 | Breast ca.* | 0.0 | 0.0 |
| | | | (pl.ef) MCF-7 | | |
| glioma U251 | 38.7 | 19.9 | Breast ca.* | 0.0 | 0.0 |
| | | | (pl.ef) MDA- | | |
| | | | MB-231 | | |
| glioma SF-295 | 4.6 | 4.1 | Breast ca.* (pl. | 10.2 | 7.8 |
| | | | ef) T47D | | |
| Heart | 100.0 | 100.0 | Breast ca. BT- | 8.4 | 5.5 |
| | | | 549 | | |
| Skeletal Muscle | 1.4 | 1.1 | Breast ca. | 0.5 | 0.1 |
| | | | MDA-N | | |
| Bone marrow | 0.3 | 0.0 | Ovary | 0.5 | 0.3 |
| Thymus | 2.1 | 1.0 | Ovarian ca. | 0.4 | 0.1 |
| | | | OVCAR-3 | | |
| Spleen | 0.0 | 0.0 | Ovarian ca. | 1.8 | 1.4 |
| | | | OVCAR-4 | | |
| Lymph node | 0.8 | 0.3 | Ovarian ca. | 39.0 | 30.1 |
| | | | OVCAR-5 | | |
| Colorectal | 0.0 | 0.0 | Ovarian ca. | 0.6 | 0.3 |
| Tissue | | | OVCAR-8 | | |
| Stomach | 1.2 | 0.7 | Ovarian ca. | 8.8 | 8.3 |
| | | | IGROV-1 | | |
| Small intestine | 0.8 | 0.4 | Ovarian ca. | 0.1 | 0.0 |
| | | | (ascites) SK- | | |
| | | | OV-3 | | |
| Colon ca. | 2.5 | 1.7 | Uterus | 0.3 | 0.2 |
| SW480 | | | | | |
| Colon ca.* | 0.0 | 0.0 | Placenta | 0.3 | 0.0 |
| SW620 (SW480 | | | | | |
| met) | | | | | |
| Colon ca. HT29 | 0.0 | 0.0 | Prostate | 5.1 | 3.2 |
| Colon ca. HCT- | 0.0 | 0.0 | Prosate ca.* | 0.1 | 0.0 |
| 116 | | | (bone met) PC-3 | | |
| Colon ca. CaCo-2 | 0.3 | 0.2 | Testis | 9.7 | 6.8 |
| Colon ca. Tissue | 0.1 | 0.0 | Melanoma | 12.0 | 10.4 |
| (ODO3866) | | | Hs688(A).T | | |
| Colon ca. HCC- | 0.0 | 0.0 | Melanoma* | 5.0 | 4.3 |
| 2998 | | | (met) | | |
| | | | Hs688(B).T | | |
| Gastric ca.* | 0.5 | 0.2 | Melanoma | 0.0 | 0.0 |
| (liver met) NCI- | | | UACC-62 | | |
| N87 | | | | | |
| Bladder | 0.9 | 0.6 | Melanoma | 0.0 | 0.0 |
| | | | M14 | | |
| Trachea | 1.2 | 0.9 | Melanoma | 0.7 | 0.2 |
| | | | LOX IMVI | | |
| Kidney | 4.0 | 2.7 | Melanoma* | 1.0 | 0.5 |
| | | | (met) SK- | | |
| | | | MEL-5 | | |
| Kidney (fetal) | 6.0 | 0.0 | | | |

TABLE AGM
Panel 1.3D

| | Rel. | Rel. | Rel. | | Rel. | Rel. | Rel. |
|---|---|---|---|---|---|---|---|
| | Exp. (%) | Exp. (%) | Exp. (%) | | Exp. (%) | Exp. (%) | Exp. (%) |
| | Ag2679, | Ag2728, | Ag2732, | | Ag2679, | Ag2728, | Ag2732, |
| Tissue | Run | Run | Run | Tissue | Run | Run | Run |
| Name | 158633802 | 158560797 | 162400878 | Name | 158633802 | 158560797 | 162400878 |
| Liver | 3.8 | 2.0 | 2.9 | Kidney | 0.2 | 1.6 | 1.2 |
| adenocarcinoma | | | | (fetal) | | | |
| Pancreas | 0.0 | 0.0 | 0.0 | Renal ca. | 9.8 | 11.5 | 3.3 |
| | | | | 786-0 | | | |
| Pancreatic | 0.0 | 0.0 | 0.0 | Renal ca. | 54.7 | 40.6 | 15.1 |
| ca. | | | | A498 | | | |
| CAPAN 2 | | | | | | | |
| Adrenal | 0.2 | 0.5 | 0.2 | Renal ca. | 11.9 | 7.3 | 8.2 |
| gland | | | | RXF 393 | | | |
| Thyroid | 0.6 | 1.3 | 0.2 | Renal ca. | 0.0 | 0.0 | 0.0 |
| | | | | ACHN | | | |
| Salivary | 0.1 | 0.3 | 0.1 | Renal ca. | 0.8 | 0.3 | 0.0 |
| gland | | | | UO-31 | | | |
| Pituitary | 0.4 | 0.3 | 0.0 | Renal ca. | 0.0 | 0.0 | 0.0 |
| gland | | | | TK-10 | | | |
| Brain | 12.4 | 15.2 | 2.5 | Liver | 0.7 | 0.4 | 0.4 |
| (fetal) | | | | | | | |
| Brain | 7.2 | 15.5 | 6.1 | Liver | 0.2 | 0.4 | 0.0 |
| (whole) | | | | (fetal) | | | |
| Brain | 11.3 | 17.4 | 7.0 | Liver ca. | 0.0 | 0.0 | 0.0 |
| (amygdala) | | | | (hepatoblast) | | | |
| | | | | HepG2 | | | |
| Brain | 1.3 | 1.0 | 0.4 | Lung | 0.1 | 0.8 | 0.1 |
| (cerebellum) | | | | | | | |
| Brain | 60.7 | 80.1 | 16.8 | Lung | 0.4 | 0.6 | 0.4 |
| (hippocampus) | | | | (fetal) | | | |
| Brain | 1.7 | 1.7 | 0.5 | Lung ca. | 0.0 | 0.0 | 0.0 |
| (substantia | | | | (small cell) | | | |
| nigra) | | | | LX-1 | | | |
| Brain | 3.7 | 10.4 | 4.5 | Lung ca. | 3.9 | 14.0 | 3.7 |
| (thalamus) | | | | (small cell) | | | |
| | | | | NCI-H69 | | | |
| Cerebral | 100.0 | 100.0 | 93.3 | Lung ca. | 0.0 | 0.2 | 0.0 |
| Cortex | | | | (s.cell var.) | | | |
| | | | | SHP-77 | | | |
| Spinal cord | 1.8 | 1.8 | 1.3 | Lung ca. | 0.2 | 0.1 | 0.5 |
| | | | | (large | | | |
| | | | | cell)NCI- | | | |
| | | | | H460 | | | |
| glio/astro | 3.0 | 4.7 | 3.0 | Lung ca. | 0.0 | 0.2 | 0.0 |
| U87-MG | | | | (non-sm. | | | |
| | | | | cell) A549 | | | |
| glio/astro | 5.3 | 5.4 | 0.8 | Lung ca. | 0.0 | 0.0 | 0.0 |
| U-118-MG | | | | (non-s.cell) | | | |
| | | | | NCI-H23 | | | |
| astrocytoma | 99.3 | 87.7 | 100.0 | Lung ca. | 6.4 | 4.3 | 3.6 |
| SW1783 | | | | (non-s.cell) | | | |
| | | | | HOP-62 | | | |
| neuro*; | 0.1 | 0.0 | 0.0 | Lung ca. | 1.3 | 1.5 | 0.9 |
| met SK-N- | | | | (non-s.cl) | | | |
| AS | | | | NCI-H522 | | | |
| astrocytoma | 80.7 | 75.3 | 42.0 | Lung ca. | 0.0 | 0.0 | 0.0 |
| SF-539 | | | | (squam.) | | | |
| | | | | SW 900 | | | |
| astrocytoma | 77.4 | 64.6 | 18.2 | Lung ca. | 1.0 | 2.5 | 2.6 |
| SNB-75 | | | | (squam.) | | | |
| | | | | NCI-H596 | | | |
| glioma | 94.0 | 75.3 | 63.7 | Mammary | 1.9 | 4.3 | 0.6 |
| SNB-19 | | | | gland | | | |
| glioma | 68.8 | 62.4 | 29.1 | Breast ca.* | 0.0 | 0.0 | 0.0 |
| U251 | | | | (pl.ef) | | | |
| | | | | MCF-7 | | | |
| glioma SF- | 8.1 | 6.9 | 4.0 | Breast ca.* | 0.8 | 0.5 | 0.1 |
| 295 | | | | (pl.ef) | | | |
| | | | | MDA-MB- | | | |
| | | | | 231 | | | |
| Heart | 34.6 | 10.6 | 11.9 | Breast ca.* | 15.9 | 7.6 | 4.9 |
| (fetal) | | | | (pl.ef) | | | |
| | | | | T47D | | | |
| Heart | 12.9 | 12.1 | 20.2 | Breast ca. | 58.2 | 36.6 | 4.0 |
| | | | | BT-549 | | | |
| Skeletal | 11.0 | 7.0 | 4.1 | Breast ca. | 0.2 | 0.0 | 0.0 |
| muscle | | | | MDA-N | | | |
| (fetal) | | | | | | | |
| Skeletal | 0.0 | 0.5 | 0.0 | Ovary | 1.8 | 1.5 | 1.4 |
| muscle | | | | | | | |
| Bone | 0.4 | 0.2 | 0.1 | Ovarian ca. | 0.0 | 0.0 | 0.0 |
| marrow | | | | OVCAR-3 | | | |
| Thymus | 0.9 | 1.2 | 3.8 | Ovarian ca. | 0.1 | 0.5 | 0.2 |
| | | | | OVCAR-4 | | | |
| Spleen | 0.0 | 0.0 | 0.0 | Ovarian ca. | 41.8 | 33.9 | 14.3 |
| | | | | OVCAR-5 | | | |
| Lymph | 0.1 | 0.6 | 0.0 | Ovarian ca. | 1.0 | 0.6 | 0.5 |
| node | | | | OVCAR-8 | | | |
| Colorectal | 1.3 | 0.5 | 0.9 | Ovarian ca. | 0.0 | 4.5 | 1.8 |
| | | | | IGROV-1 | | | |
| Stomach | 0.4 | 0.5 | 0.1 | Ovarian | 0.1 | 0.1 | 0.0 |
| | | | | ca.* | | | |
| | | | | (ascites) | | | |
| | | | | SK-OV-3 | | | |
| Small | 0.0 | 0.6 | 0.2 | Uterus | 0.0 | 0.0 | 0.0 |
| intestine | | | | | | | |
| Colon ca. | 11.2 | 5.4 | 1.1 | Placenta | 0.1 | 0.0 | 0.0 |
| SW480 | | | | | | | |
| Colon ca.* | 0.0 | 0.0 | 0.0 | Prostate | 1.2 | 0.5 | 0.6 |
| SW620(SW480 | | | | | | | |
| met) | | | | | | | |
| Colon ca. | 0.0 | 0.0 | 0.0 | Prostate | 0.5 | 0.0 | 0.0 |
| HT29 | | | | ca.* (bone | | | |
| | | | | met)PC-3 | | | |
| Colon ca. | 0.0 | 0.0 | 0.0 | Testis | 1.8 | 1.4 | 0.9 |
| HCT-116 | | | | | | | |
| Colon ca. | 0.4 | 0.1 | 0.1 | Melanoma | 42.3 | 22.4 | 17.1 |
| CaCo-2 | | | | Hs688(A).T | | | |
| Colon ca. | 0.4 | 0.4 | 0.3 | Melanoma* | 1.7 | 1.6 | 1.0 |
| tissue(ODO3866) | | | | (met) | | | |
| | | | | Hs688(B).T | | | |
| Colon ca. | 0.0 | 0.0 | 0.0 | Melanoma | 0.0 | 0.0 | 0.0 |
| HCC-2998 | | | | UACC-62 | | | |
| Gastric | 0.2 | 0.4 | 0.1 | Melanoma | 0.0 | 0.0 | 0.0 |
| ca.* (liver | | | | M14 | | | |
| met) NCI- | | | | | | | |
| N87 | | | | | | | |
| Bladder | 0.5 | 0.3 | 0.3 | Melanoma | 2.4 | 1.1 | 0.4 |
| | | | | LOX IMVI | | | |
| Trachea | 1.3 | 1.5 | 1.0 | Melanoma* | 0.0 | 0.0 | 0.0 |
| | | | | (met) | | | |
| | | | | SK-MEL-5 | | | |
| Kidney | 0.7 | 0.3 | 0.2 | Adipose | 0.2 | 0.7 | 0.0 |

TABLE AGN
Panel 2.2

| | Rel. Exp. (%) | | Rel. Exp. (%) |
|---|---|---|---|
| | Ag2975, Run | | Ag2975, Run |
| Tissue Name | 173763053 | Tissue Name | 173763053 |
| Normal Colon | 2.5 | Kidney Margin | 29.7 |
| | | (OD04348) | |
| Colon cancer | 4.7 | Kidney malignant | 0.0 |
| (OD06064) | | cancer (OD06204B) | |
| Colon Margin | 4.3 | Kidney normal adjacent | 0.0 |
| (OD06064) | | tissue (OD06204E) | |
| Colon cancer | 2.2 | Kidney Cancer | 0.0 |
| (OD06159) | | (OD04450-01) | |
| Colon Margin | 7.3 | Kidney Margin | 13.6 |
| (OD06159) | | (OD04450-03) | |
| Colon cancer | 0.0 | Kidney Cancer 8120613 | 2.3 |
| (OD06297-04) | | | |
| Colon Margin | 0.0 | Kidney Margin | 11.5 |
| (OD06297-015) | | 8120614 | |
| CC Gr.2 ascend colon | 0.0 | Kidney Cancer 9010320 | 23.8 |
| (ODO3921) | | | |
| CC Margin (ODO3921) | 0.0 | Kidney Margin | 20.0 |
| | | 9010321 | |
| Colon cancer metastasis | 0.0 | Kidney Cancer 8120607 | 2.0 |
| (OD06104) | | | |
| Lung Margin | 0.0 | Kidney Margin | 0.0 |
| (OD06104) | | 8120608 | |
| Colon mets to lung | 0.0 | Normal Uterus | 4.4 |
| (OD04451-01) | | | |
| Lung Margin | 2.0 | Uterine Cancer 064011 | 0.0 |
| (OD04451-02) | | | |
| Normal Prostate | 7.5 | Normal Thyroid | 4.4 |
| Prostate Cancer | 2.3 | Thyroid Cancer 064010 | 0.0 |
| (OD04410) | | | |
| Prostate Margin | 4.6 | Thyroid Cancer | 2.2 |
| (OD04410) | | A302152 | |
| Normal Ovary | 2.2 | Thyroid Margin | 3.6 |
| | | A302153 | |
| Ovarian cancer | 0.0 | Normal Breast | 55.1 |
| (OD06283-03) | | | |
| Ovarian Margin | 0.0 | Breast Cancer | 3.9 |
| (OD06283-07) | | (OD04566) | |
| Ovarian Cancer 064008 | 13.4 | Breast Cancer 1024 | 68.3 |
| Ovarian cancer | 18.8 | Breast Cancer | 5.3 |
| (OD06145) | | (OD04590-01) | |
| Ovarian Margin | 8.1 | Breast Cancer Mets | 0.0 |
| (OD06145) | | (OD04590-03) | |
| Ovarian cancer | 5.0 | Breast Cancer | 13.1 |
| (OD06455-03) | | Metastasis (OD04655- | |
| | | 05) | |
| Ovarian Margin | 2.3 | Breast Cancer 064006 | 12.5 |
| (OD06455-07) | | | |
| Normal Lung | 2.3 | Breast Cancer 9100266 | 34.6 |
| Invasive poor diff. lung | 0.0 | Breast Margin 9100265 | 17.0 |
| adeno (ODO4945-01 | | | |
| Lung Margin | 0.0 | Breast Cancer A209073 | 16.7 |
| (ODO4945-03) | | | |
| Lung Malignant Cancer | 1.4 | Breast Margin | 71.7 |
| (OD03126) | | A2090734 | |
| Lung Margin | 0.0 | Breast cancer | 20.7 |
| (OD03126) | | (OD06083) | |
| Lung Cancer | 0.0 | Breast cancer node | 6.3 |
| (OD05014A) | | metastasis (OD06083) | |
| Lung Margin | 2.1 | Normal Liver | 0.0 |
| (OD05014B) | | | |
| Lung cancer (OD06081) | 100.0 | Liver Cancer 1026 | 8.1 |
| Lung Margin | 4.1 | Liver Cancer 1025 | 29.5 |
| (OD06081) | | | |
| Lung Cancer | 0.0 | Liver Cancer 6004-T | 8.4 |
| (OD04237-01) | | | |
| Lung Margin | 0.0 | Liver Tissue 6004-N | 4.7 |
| (OD04237-02) | | | |
| Ocular Melanoma | 6.7 | Liver Cancer 6005-T | 23.0 |
| Metastasis | | | |
| Ocular Melanoma | 2.8 | Liver Tissue 6005-N | 32.8 |
| Margin (Liver) | | | |
| Melanoma Metastasis | 2.2 | Liver Cancer 064003 | 12.8 |
| Melanoma Margin | 3.7 | Normal Bladder | 0.0 |
| (Lung) | | | |
| Normal Kidney | 9.0 | Bladder Cancer 1023 | 6.3 |
| Kidney Ca, Nuclear | 19.5 | Bladder Cancer | 16.7 |
| grade 2 (OD04338) | | A302173 | |
| Kidney Margin | 3.1 | Normal Stomach | 15.3 |
| (OD04338) | | | |
| Kidney Ca Nuclear | 0.0 | Gastric Cancer 9060397 | 0.0 |
| grade 1/2 (OD04339) | | | |
| Kidney Margin | 9.3 | Stomach Margin | 3.8 |
| (OD04339) | | 9060396 | |
| Kidney Ca, Clear cell | 0.0 | Gastric Cancer 9060395 | 6.5 |
| type (OD04340) | | | |
| Kidney Margin | 4.6 | Stomach Margin 9060394 | 2.3 |
| (OD04340) | | | |
| Kidney Ca, Nuclear | 77.4 | Gastric Cancer 064005 | 0.0 |
| grade 3 (OD04348) | | | |

TABLE AGO
Panel 2D

| | Rel. Exp. (%) | Rel. Exp. (%) | Rel. Exp. (%) | Rel. Exp. (%) |
|---|---|---|---|---|
| | Ag047, Run | Ag047, Run | Ag2679, Run | Ag2728, Run |
| Tissue Name | 144771648 | 152940364 | 158633803 | 158561830 |
| Normal Colon | 5.5 | 8.9 | 7.4 | 10.4 |
| CC Well to Mod | 1.4 | 0.2 | 0.2 | 1.3 |
| Diff (ODO3866) | | | | |
| CC Margin | 0.4 | 0.5 | 0.4 | 0.1 |
| (ODO3866) | | | | |
| CC Gr.2 | 0.4 | 0.1 | 1.7 | 0.2 |
| rectosigmoid | | | | |
| (ODO3868) | | | | |
| CC Margin | 2.5 | 0.9 | 0.7 | 0.7 |
| (ODO3868) | | | | |
| CC Mod Diff | 0.0 | 0.0 | 0.0 | 0.2 |
| (ODO3920) | | | | |
| CC Margin | 0.7 | 0.9 | 1.1 | 0.3 |
| (ODO3920) | | | | |
| CC Gr.2 ascend | 0.0 | 0.7 | 0.6 | 0.2 |
| colon (ODO3921) | | | | |
| CC Margin | 0.2 | 0.2 | 0.6 | 0.8 |
| (ODO3921) | | | | |
| CC from Partial | 0.6 | 0.4 | 1.1 | 1.0 |
| Hepatectomy | | | | |
| (ODO4309) Mets | | | | |
| Liver Margin | 11.1 | 13.6 | 30.4 | 21.2 |
| (ODO4309) | | | | |
| Colon mets to lung | 0.0 | 0.4 | 0.1 | 0.2 |
| (OD04451-01) | | | | |
| Lung Margin | 1.3 | 1.0 | 0.5 | 0.7 |
| (OD04451-02) | | | | |
| Normal Prostate | 19.1 | 0.7 | 1.0 | 2.3 |
| 6546-1 | | | | |
| Prostate Cancer | 5.8 | 2.9 | 4.7 | 3.4 |
| (OD04410) | | | | |
| Prostate Margin | 4.7 | 5.7 | 7.2 | 4.9 |
| (OD04410) | | | | |
| Prostate Cancer | 2.9 | 3.3 | 3.4 | 3.3 |
| (OD04720-01) | | | | |
| Prostate Margin | 12.5 | 10.7 | 8.3 | 15.2 |
| (OD04720-02) | | | | |
| Normal Lung | 1.5 | 2.7 | 6.8 | 5.4 |
| 061010 | | | | |
| Lung Met to Muscle | 0.3 | 0.1 | 0.0 | 0.5 |
| (ODO4286) | | | | |
| Muscle Margin | 0.3 | 0.2 | 0.3 | 0.6 |
| (ODO4286) | | | | |
| Lung Malignant | 0.4 | 0.2 | 0.8 | 1.5 |
| Cancer (OD03126) | | | | |
| Lung Margin | 0.4 | 1.0 | 1.2 | 0.5 |
| (OD03126) | | | | |
| Lung Cancer | 86.5 | 100.0 | 100.0 | 100.0 |
| (OD04404) | | | | |
| Lung Margin | 18.3 | 3.3 | 2.2 | 3.0 |
| (OD04404) | | | | |
| Lung Cancer | 100.0 | 52.1 | 62.0 | 77.9 |
| (OD04565) | | | | |
| Lung Margin | 0.2 | 0.1 | 0.0 | 0.6 |
| (OD04565) | | | | |
| Lung Cancer | 6.3 | 1.5 | 3.2 | 3.3 |
| (OD04237-01) | | | | |
| Lung Margin | 1.4 | 0.5 | 0.5 | 0.6 |
| (OD04237-02) | | | | |
| Ocular Mel Met to | 0.4 | 0.3 | 0.5 | 0.7 |
| Liver (ODO4310) | | | | |
| Liver Margin | 2.3 | 1.8 | 3.5 | 2.8 |
| (ODO4310) | | | | |
| Melanoma Mets to | 0.0 | 0.3 | 0.4 | 1.6 |
| Lung (OD04321) | | | | |
| Lung Margin | 2.1 | 2.6 | 2.2 | 3.0 |
| (OD04321) | | | | |
| Normal Kidney | 6.7 | 4.9 | 8.4 | 7.0 |
| Kidney Ca, Nuclear | 0.0 | 0.0 | 0.3 | 1.4 |
| grade 2 (OD04338) | | | | |
| Kidney Margin | 3.5 | 1.5 | 4.1 | 3.5 |
| (OD04338) | | | | |
| Kidney Ca Nuclear | 0.0 | 0.1 | 0.3 | 0.7 |
| grade 1/2 | | | | |
| (OD04339) | | | | |
| Kidney Margin | 18.4 | 10.3 | 8.4 | 15.5 |
| (OD04339) | | | | |
| Kidney Ca, Clear | 0.0 | 0.8 | 0.7 | 1.1 |
| cell type (OD04340) | | | | |
| Kidney Margin | 6.5 | 4.4 | 4.5 | 6.5 |
| (OD04340) | | | | |
| Kidney Ca, Nuclear | 90.8 | 36.1 | 54.3 | 50.3 |
| grade 3 (OD04348) | | | | |
| Kidney Margin | 4.6 | 3.2 | 3.8 | 4.0 |
| (OD04348) | | | | |
| Kidney Cancer | 2.7 | 1.8 | 4.1 | 3.5 |
| (OD04622-01) | | | | |
| Kidney Margin | 2.0 | 0.2 | 0.3 | 1.1 |
| (OD04622-03) | | | | |
| Kidney Cancer | 0.0 | 0.0 | 0.0 | 0.0 |
| (OD04450-01) | | | | |
| Kidney Margin | 3.1 | 1.4 | 6.9 | 5.4 |
| (OD04450-03) | | | | |
| Kidney Cancer | 1.5 | 0.3 | 0.5 | 1.8 |
| 8120607 | | | | |
| Kidney Margin | 3.4 | 0.8 | 1.3 | 2.2 |
| 8120608 | | | | |
| Kidney Cancer | 0.4 | 0.8 | 4.3 | 2.0 |
| 8120613 | | | | |
| Kidney Margin | 2.8 | 1.2 | 4.9 | 4.0 |
| 8120614 | | | | |
| Kidney Cancer | 76.8 | 39.0 | 36.3 | 52.9 |
| 9010320 | | | | |
| Kidney Margin | 10.4 | 5.1 | 4.9 | 4.3 |
| 9010321 | | | | |
| Normal Uterus | 0.0 | 0.2 | 0.3 | 0.0 |
| Uterus Cancer | 0.9 | 0.0 | 0.3 | 1.9 |
| 064011 | | | | |
| Normal Thyroid | 0.3 | 0.0 | 0.9 | 2.2 |
| Thyroid Cancer | 0.0 | 0.0 | 0.0 | 0.0 |
| 064010 | | | | |
| Thyroid Cancer | 1.4 | 0.1 | 0.4 | 0.0 |
| A302152 | | | | |
| Thyroid Margin | 1.7 | 0.9 | 6.4 | 4.1 |
| A302153 | | | | |
| Normal Breast | 20.3 | 5.7 | 13.0 | 20.9 |
| Breast Cancer | 0.3 | 0.1 | 0.5 | 0.3 |
| (OD04566) | | | | |
| Breast Cancer | 2.4 | 2.2 | 1.1 | 1.9 |
| (OD04590-01) | | | | |
| Breast Cancer Mets | 0.7 | 0.1 | 0.3 | 0.0 |
| (OD04590-03) | | | | |
| Breast Cancer | 6.9 | 3.3 | 7.7 | 9.3 |
| Metastasis | | | | |
| (OD04655-05) | | | | |
| Breast Cancer | 5.6 | 8.3 | 4.2 | 4.9 |
| 064006 | | | | |
| Breast Cancer 1024 | 47.0 | 19.3 | 30.1 | 23.5 |
| Breast Cancer | 14.9 | 9.3 | 15.7 | 21.8 |
| 9100266 | | | | |
| Breast Margin | 4.6 | 1.5 | 4.4 | 8.4 |
| 9100265 | | | | |
| Breast Cancer | 48.3 | 12.9 | 28.3 | 40.9 |
| A209073 | | | | |
| Breast Margin | 38.7 | 16.6 | 27.5 | 29.7 |
| A2090734 | | | | |
| Normal Liver | 0.2 | 0.1 | 0.0 | 0.2 |
| Liver Cancer 064003 | 11.8 | 5.6 | 4.6 | 4.5 |
| Liver Cancer 1025 | 4.5 | 1.6 | 4.2 | 2.8 |
| Liver Cancer 1026 | 6.2 | 6.7 | 9.0 | 6.4 |
| Liver Cancer 6004-T | 15.6 | 3.3 | 3.5 | 2.8 |
| Liver Tissue 6004-N | 0.1 | 0.2 | 0.2 | 0.4 |
| Liver Cancer 6005-T | 14.6 | 8.0 | 8.7 | 7.9 |
| Liver Tissue 6005-N | 6.4 | 7.0 | 5.9 | 3.2 |
| Normal Bladder | 1.3 | 0.9 | 1.6 | 0.9 |
| Bladder Cancer 1023 | 0.4 | 0.2 | 0.3 | 0.3 |
| Bladder Cancer | 7.5 | 5.3 | 12.1 | 19.8 |
| A302173 | | | | |
| Bladder Cancer | 27.7 | 23.0 | 35.6 | 41.5 |
| (OD04718-01) | | | | |
| Bladder Normal | 0.4 | 0.3 | 0.6 | 0.7 |
| Adjacent (OD04718- | | | | |
| 03) | | | | |
| Normal Ovary | 1.4 | 0.4 | 1.1 | 0.3 |
| Ovarian Cancer | 1.3 | 0.5 | 1.3 | 1.3 |
| 064008 | | | | |
| Ovarian Cancer | 0.4 | 0.2 | 0.0 | 0.0 |
| (OD04768-07) | | | | |
| Ovary Margin | 1.0 | 0.8 | 0.6 | 1.6 |
| (OD04768-08) | | | | |
| Normal Stomach | 4.3 | 2.0 | 0.3 | 3.1 |
| Gastric Cancer | 2.4 | 0.7 | 0.7 | 1.2 |
| 9060358 | | | | |
| Stomach Margin | 0.0 | 0.0 | 0.7 | 4.3 |
| 9060359 | | | | |
| Gastric Cancer | 0.9 | 2.0 | 1.9 | 2.4 |
| 9060395 | | | | |
| Stomach Margin | 0.2 | 0.3 | 1.1 | 0.2 |
| 9060394 | | | | |
| Gastric Cancer | 0.4 | 0.5 | 1.6 | 0.7 |
| 9060397 | | | | |
| Stomach Margin | 0.0 | 0.1 | 0.2 | 0.2 |
| 9060396 | | | | |
| Gastric Cancer | 0.7 | 0.7 | 0.9 | 1.5 |
| 064005 | | | | |

TABLE AGP
Panel 3D

| | Rel. Exp. (%) | | Rel. Exp. (%) |
|---|---|---|---|
| | Ag047, Run | | Ag047, Run |
| Tissue Name | 158634002 | Tissue Name | 158634002 |
| Daoy-Medulloblastoma | 0.9 | Ca Ski-Cervical epidermoid | 0.3 |
| | | carcinoma (metastasis) | |
| TE671-Medulloblastoma | 0.0 | ES-2-Ovarian clear cell | 6.7 |
| | | carcinoma | |
| D283 Med- | 0.0 | Ramos-Stimulated with | 0.0 |
| Medulloblastoma | | PMA/ionomycin 6 h | |
| PFSK-1-Primitive | 0.0 | Ramos-Stimulated with | 0.0 |
| Neuroectodermal | | PMA/ionomycin 14 h | |
| XF-498-CNS | 0.0 | MEG-01-Chronic | 0.0 |
| | | myelogenous leukemia | |
| | | (megokaryoblast) | |
| SNB-78-Glioma | 100.0 | Raji-Burkitt's lymphoma | 0.0 |
| SF-268-Glioblastoma | 79.0 | Daudi-Burkitt's lymphoma | 0.0 |
| T98G-Glioblastoma | 1.4 | U266-B-cell plasmacytoma | 0.0 |
| SK-N-SH- | 2.0 | CA46-Burkitt's lymphoma | 0.0 |
| Neuroblastoma | | | |
| (metastasis) | | | |
| SF-295-Glioblastoma | 0.1 | RL-non-Hodgkin's B-cell | 0.0 |
| | | lymphoma | |
| Cerebellum | 4.5 | JM1-pre-B-cell lymphoma | 0.0 |
| Cerebellum | 2.9 | Jurkat-T cell leukemia | 0.0 |
| NCI-H292- | 3.8 | TF-1-Erythroleukemia | 0.0 |
| Mucoepidermoid lung | | | |
| carcinoma | | | |
| DMS-114-Small cell | 0.0 | HUT 78-T-cell lymphoma | 0.0 |
| lung cancer | | | |
| DMS-79-Small cell lung | 0.1 | U937-Histiocytic lymphoma | 0.0 |
| cancer | | | |
| NCI-H146-Small cell | 0.0 | KU-812-Myelogenous | 0.0 |
| lung cancer | | leukemia | |
| NCI-H526-Small cell | 1.0 | 769-P-Clear cell renal | 0.1 |
| lung cancer | | carcinoma | |
| NCI-N417-Small cell | 0.0 | Caki-2-Clear cell renal | 0.1 |
| lung cancer | | carcinoma | |
| NCI-H82-Small cell lung | 7.6 | SW 839-Clear cell renal | 0.0 |
| cancer | | carcinoma | |
| NCI-H157-Squamous | 0.8 | G401-Wilms' tumor | 6.0 |
| cell lung cancer | | | |
| (metastasis) | | | |
| NCI-H1155-Large cell | 0.0 | Hs766T-Pancreatic | 0.0 |
| lung cancer | | carcinoma (LN metastasis) | |
| NCI-H1299-Large cell | 0.2 | CAPAN-1-Pancreatic | 0.0 |
| lung cancer | | adenocarcinoma (liver | |
| | | metastasis) | |
| NCI-H727-Lung | 0.0 | SU86.86-Pancreatic | 0.1 |
| carcinoid | | carcinoma (liver metastasis) | |
| NCI-UMC-11-Lung | 0.0 | BxPC-3-Pancreatic | 0.6 |
| carcinoid | | adenocarcinoma | |
| LX-1-Small cell lung | 0.0 | HPAC-Pancreatic | 0.0 |
| cancer | | adenocarcinoma | |
| Colo-205-Colon cancer | 0.0 | MIA PaCa-2-Pancreatic | 0.0 |
| | | carcinoma | |
| KM12-Colon cancer | 0.0 | CFPAC-1-Pancreatic ductal | 0.1 |
| | | adenocarcinoma | |
| KM20L2-Colon cancer | 0.0 | PANC-1-Pancreatic | 0.0 |
| | | epithelioid ductal carcinoma | |
| NCI-H716-Colon cancer | 0.0 | T24-Bladder carcinma | 0.0 |
| | | (transitional cell) | |
| SW-48-Colon | 0.0 | 5637-Bladder carcinoma | 3.8 |
| adenocarcinoma | | | |
| SW1116-Colon | 0.0 | HT-1197-Bladder carcinoma | 0.0 |
| adenocarcinoma | | | |
| LS 174T-Colon | 0.0 | UM-UC-3-Bladder carcinma | 10.3 |
| adenocarcinoma | | (transitional cell) | |
| SW-948-Colon | 0.0 | A204-Rhabdomyosarcoma | 0.0 |
| adenocarcinoma | | | |
| SW-480-Colon | 0.0 | HT-1080-Fibrosarcoma | 59.9 |
| adenocarcinoma | | | |
| NCI-SNU-5-Gastric | 0.7 | MG-63-Osteosarcoma | 0.5 |
| carcinoma | | | |
| KATO III-Gastric | 0.0 | SK-LMS-1-Leiomyosarcoma | 4.2 |
| carcinoma | | (vulva) | |
| NCI-SNU-16-Gastric | 0.2 | SJRH30-Rhabdomyosarcoma | 0.3 |
| carcinoma | | (met to bone marrow) | |
| NCI-SNU-1-Gastric | 0.0 | A431-Epidermoid carcinoma | 0.6 |
| carcinoma | | | |
| RF-1-Gastric | 0.3 | WM266-4-Melanoma | 0.0 |
| adenocarcinoma | | | |
| RF-48-Gastric | 0.1 | DU 145-Prostate carcinoma | 0.0 |
| adenocarcinoma | | (brain metastasis) | |
| MKN-45-Gastric | 0.0 | MDA-MB-468-Breast | 0.0 |
| carcinoma | | adenocarcinoma | |
| NCI-N87-Gastric | 0.0 | SCC-4-Squamous cell | 0.2 |
| carcinoma | | carcinoma of tongue | |
| OVCAR-5-Ovarian | 0.0 | SCC-9-Squamous cell | 1.1 |
| carcinoma | | carcinoma of tongue | |
| RL95-2-Uterine | 4.0 | SCC-15-Squamous cell | 0.3 |
| carcinoma | | carcinoma of tongue | |
| HelaS3-Cervical | 0.0 | CAL 27-Squamous cell | 7.0 |
| adenocarcinoma | | carcinoma of tongue | |

TABLE AGQ
Panel 4.1D

| | Rel. Exp. (%) | | Rel. Exp. (%) |
|---|---|---|---|
| | Ag2975, Run | | Ag2975, Run |
| Tissue Name | 171818669 | Tissue Name | 171818669 |
| Secondary Th1 act | 0.0 | HUVEC IL-1beta | 0.0 |
| Secondary Th2 act | 0.0 | HUVEC IFN gamma | 0.0 |
| Secondary Tr1 act | 0.0 | HUVEC TNF alpha + IFN | 0.0 |
| | | gamma | |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC | 0.0 |
| | | none | |
| Primary Th1 act | 0.0 | Lung Microvascular EC | 1.1 |
| | | TNF alpha + IL-1beta | |
| Primary Th2 act | 0.0 | Microvascular Dermal EC | 0.0 |
| | | none | |
| Primary Tr1 act | 0.0 | Microsvasular Dermal EC | 0.0 |
| | | TNF alpha + IL-1beta | |
| Primary Th1 rest | 0.0 | Bronchial epithelium | 37.4 |
| | | TNF alpha + IL1beta | |
| Primary Th2 rest | 0.0 | Small airway epithelium | 47.6 |
| | | none | |
| Primary Tr1 rest | 0.0 | Small airway epithelium | 100.0 |
| | | TNF alpha + IL-1beta | |
| CD45RA CD4 | 0.8 | Coronery artery SMC rest | 0.8 |
| lymphocyte act | | | |
| CD45RO CD4 | 0.0 | Coronery artery SMC | 0.6 |
| lymphocyte act | | TNF alpha + IL-1beta | |
| CD8 lymphocyte act | 0.0 | Astrocytes rest | 51.8 |
| Secondary CD8 | 0.1 | Astrocytes TNF alpha + | 78.5 |
| lymphocyte rest | | IL-1beta | |
| Secondary CD8 | 0.0 | KU-812 (Basophil) rest | 0.0 |
| lymphocyte act | | | |
| CD4 lymphocyte none | 0.0 | KU-812 (Basophil) | 0.0 |
| | | PMA/ionomycin | |
| 2ry Th1/Th2/Tr1_anti- | 0.0 | CCD1106 (Keratinocytes) | 45.1 |
| CD95 CH11 | | none | |
| LAK cells rest | 0.0 | CCD1106 (Keratinocytes) | 33.7 |
| | | TNF alpha + IL-1beta | |
| LAK cells IL-2 | 0.0 | Liver cirrhosis | 0.2 |
| LAK cells IL-2 + IL-12 | 0.0 | NCI-H292 none | 0.5 |
| LAK cells IL-2 + IFN | 0.0 | NCI-H292 IL-4 | 12.6 |
| gamma | | | |
| LAK cells IL-2 + IL-18 | 0.1 | NCI-H292 IL-9 | 0.6 |
| LAK cells | 0.0 | NCI-H292 IL-13 | 10.9 |
| PMA/ionomycin | | | |
| NK Cells IL-2 rest | 0.0 | NCI-H292 IFN gamma | 0.9 |
| Two Way MLR 3 day | 0.0 | HPAEC none | 1.2 |
| Two Way MLR 5 day | 0.0 | HPAEC TNF alpha + IL- | 0.3 |
| | | 1beta | |
| Two Way MLR 7 day | 0.1 | Lung fibroblast none | 1.5 |
| PBMC rest | 0.0 | Lung fibroblast TNF alpha + | 0.0 |
| | | IL-1beta | |
| PBMC PWM | 0.0 | Lung fibroblast IL-4 | 0.1 |
| PBMC PHA-L | 0.0 | Lung fibroblast IL-9 | 0.2 |
| Ramos (B cell) none | 0.0 | Lung fibroblast IL-13 | 1.3 |
| Ramos (B cell) | 0.0 | Lung fibroblast IFN | 0.8 |
| ionomycin | | gamma | |
| B lymphocytes PWM | 0.0 | Dermal fibroblast | 4.1 |
| | | CCD1070 rest | |
| B lymphocytes CD40L | 0.0 | Dermal fibroblast | 2.2 |
| and IL-4 | | CCD1070 TNF alpha | |
| EOL-1 dbcAMP | 0.0 | Dermal fibroblast | 0.8 |
| | | CCD1070 IL-1beta | |
| EOL-1 dbcAMP | 0.0 | Dermal fibroblast IFN | 2.4 |
| PMA/ionomycin | | gamma | |
| Dendritic cells none | 0.0 | Dermal fibroblast IL-4 | 5.4 |
| Dendritic cells LPS | 0.0 | Dermal Fibroblast rest | 9.3 |
| Dendritic cells anti- | 0.1 | Neutrophils TNFa + LPS | 0.6 |
| CD40 | | | |
| Monocytes rest | 0.0 | Neutrophils rest | 0.0 |
| Monocytes LPS | 0.0 | Colon | 0.5 |
| Macrophages rest | 0.0 | Lung | 1.6 |
| Macrophages LPS | 0.0 | Thymus | 9.3 |
| HUVEC none | 0.0 | Kidney | 11.3 |
| HUVEC starved | 0.4 | | |

TABLE AGR
Panel 4D

| | Rel. | Rel. | Rel. | Rel. | Rel. | Rel. | Rel. |
|---|---|---|---|---|---|---|---|
| | Exp. (%) | Exp. (%) | Exp. (%) | Exp. (%) | Exp. (%) | Exp. (%) | Exp. (%) |
| | Ag047, | Ag047, | Ag047, | Ag2679, | Ag2728, | Ag2975, | Ag47b, |
| Tissue | Run | Run | Run | Run | Run | Run | Run |
| Name | 146087309 | 151918119 | 152893522 | 158535667 | 158562582 | 164314612 | 158664022 |
| Secondary | 0.0 | 0.0 | 0.0 | 0.0 | 0.0 | 0.0 | 0.1 |
| Th1 act | | | | | | | |
| Secondary | 0.0 | 0.0 | 0.0 | 0.0 | 0.0 | 0.0 | 0.0 |
| Th2 act | | | | | | | |
| Secondary | 0.0 | 0.0 | 0.0 | 0.0 | 0.0 | 0.0 | 0.0 |
| Tr1 act | | | | | | | |
| Secondary | 0.0 | 0.0 | 0.0 | 0.0 | 0.0 | 0.0 | 0.0 |
| Th1 rest | | | | | | | |
| Secondary | 0.0 | 0.0 | 0.0 | 0.0 | 0.0 | 0.0 | 0.0 |
| Th2 rest | | | | | | | |
| Secondary | 0.0 | 0.0 | 0.0 | 0.0 | 0.0 | 0.0 | 0.0 |
| Tr1 rest | | | | | | | |
| Primary | 0.0 | 0.0 | 0.0 | 0.0 | 0.0 | 0.0 | 0.0 |
| Th1 act | | | | | | | |
| Primary | 0.0 | 0.0 | 0.0 | 0.0 | 0.0 | 0.0 | 0.0 |
| Th2 act | | | | | | | |
| Primary | 0.0 | 0.0 | 0.0 | 0.0 | 0.0 | 0.0 | 0.0 |
| Tr1 act | | | | | | | |
| Primary | 0.0 | 0.0 | 0.0 | 0.0 | 0.0 | 0.0 | 0.0 |
| Th1 rest | | | | | | | |
| Primary | 0.0 | 0.0 | 0.0 | 0.0 | 0.0 | 0.0 | 0.0 |
| Th2 rest | | | | | | | |
| Primary | 0.0 | 0.0 | 0.0 | 0.0 | 0.0 | 0.0 | 0.0 |
| Tr1 rest | | | | | | | |
| CD45RA | 0.1 | 0.2 | 0.1 | 0.3 | 0.3 | 0.0 | 0.3 |
| CD4 | | | | | | | |
| lymphocyte | | | | | | | |
| act | | | | | | | |
| CD45RO | 0.0 | 0.0 | 0.0 | 0.0 | 0.0 | 0.0 | 0.1 |
| CD4 | | | | | | | |
| lymphocyte | | | | | | | |
| act | | | | | | | |
| CD8 | 0.0 | 0.0 | 0.0 | 0.0 | 0.0 | 0.0 | 0.0 |
| lymphocyte | | | | | | | |
| act | | | | | | | |
| Secondary | 0.0 | 0.0 | 0.0 | 0.0 | 0.0 | 0.0 | 0.0 |
| CD8 | | | | | | | |
| lymphocyte | | | | | | | |
| rest | | | | | | | |
| Secondary | 0.0 | 0.0 | 0.0 | 0.0 | 0.0 | 0.0 | 0.0 |
| CD8 | | | | | | | |
| lymphocyte | | | | | | | |
| act | | | | | | | |
| CD4 | 0.0 | 0.0 | 0.0 | 0.0 | 0.0 | 0.0 | 0.0 |
| lymphocyte | | | | | | | |
| none | | | | | | | |
| 2ry | 0.0 | 0.0 | 0.0 | 0.0 | 0.0 | 0.0 | 0.0 |
| Th1/Th2/Tr1_{—} | | | | | | | |
| anti- | | | | | | | |
| CD95 | | | | | | | |
| CH11 | | | | | | | |
| LAK cells | 0.0 | 0.0 | 0.0 | 0.0 | 0.0 | 0.0 | 0.0 |
| rest | | | | | | | |
| LAK cells | 0.0 | 0.0 | 0.0 | 0.0 | 0.0 | 0.0 | 0.0 |
| IL-2 | | | | | | | |
| LAK cells | 0.0 | 0.0 | 0.0 | 0.0 | 0.0 | 0.0 | 0.0 |
| IL-2 + IL- | | | | | | | |
| 12 | | | | | | | |
| LAK cells | 0.1 | 0.0 | 0.0 | 0.0 | 0.0 | 0.0 | 0.1 |
| IL-2 + IFN | | | | | | | |
| gamma | | | | | | | |
| LAK cells | 0.0 | 0.0 | 0.0 | 0.0 | 0.1 | 0.0 | 0.0 |
| IL-2 + IL- | | | | | | | |
| 18 | | | | | | | |
| LAK cells | 0.0 | 0.0 | 0.0 | 0.0 | 0.0 | 0.0 | 0.0 |
| PMA/ionomycin | | | | | | | |
| NK Cells | 0.0 | 0.0 | 0.0 | 0.0 | 0.0 | 0.0 | 0.0 |
| IL-2 rest | | | | | | | |
| Two Way | 0.0 | 0.0 | 0.0 | 0.0 | 0.0 | 0.0 | 0.0 |
| MLR 3 | | | | | | | |
| day | | | | | | | |
| Two Way | 0.0 | 0.0 | 0.0 | 0.0 | 0.0 | 0.0 | 0.0 |
| MLR 5 | | | | | | | |
| day | | | | | | | |
| Two Way | 0.0 | 0.0 | 0.0 | 0.0 | 0.0 | 0.0 | 0.0 |
| MLR 7 | | | | | | | |
| day | | | | | | | |
| PBMC rest | 0.0 | 0.0 | 0.0 | 0.0 | 0.0 | 0.0 | 0.0 |
| PBMC | 0.0 | 0.0 | 0.0 | 0.0 | 0.1 | 0.0 | 0.0 |
| PWM | | | | | | | |
| PBMC | 0.0 | 0.0 | 0.0 | 0.0 | 0.0 | 0.0 | 0.0 |
| PHA-L | | | | | | | |
| Ramos (B | 0.0 | 0.0 | 0.0 | 0.0 | 0.0 | 0.0 | 0.0 |
| cell) none | | | | | | | |
| Ramos (B | 0.0 | 0.0 | 0.0 | 0.0 | 0.0 | 0.0 | 0.0 |
| cell) | | | | | | | |
| ionomycin | | | | | | | |
| B | 0.0 | 0.0 | 0.0 | 0.0 | 0.0 | 0.0 | 0.0 |
| lymphocytes | | | | | | | |
| PWM | | | | | | | |
| B | 0.0 | 0.0 | 0.0 | 0.0 | 0.1 | 0.0 | 0.0 |
| lymphocytes | | | | | | | |
| CD40L | | | | | | | |
| and IL-4 | | | | | | | |
| EOL-1 | 0.0 | 0.0 | 0.0 | 0.0 | 0.0 | 0.0 | 0.1 |
| dbcAMP | | | | | | | |
| EOL-1 | 0.0 | 0.0 | 0.0 | 0.0 | 0.0 | 0.0 | 0.0 |
| dbcAMP | | | | | | | |
| PMA/ionomycin | | | | | | | |
| Dendritic | 0.0 | 0.0 | 0.0 | 0.0 | 0.0 | 0.0 | 0.0 |
| cells none | | | | | | | |
| Dendritic | 0.0 | 0.0 | 0.0 | 0.0 | 0.0 | 0.0 | 0.0 |
| cells LPS | | | | | | | |
| Dendritic | 0.0 | 0.0 | 0.0 | 0.0 | 0.0 | 0.0 | 0.0 |
| cells anti- | | | | | | | |
| CD40 | | | | | | | |
| Monocytes | 0.0 | 0.0 | 0.0 | 0.0 | 0.0 | 0.0 | 0.0 |
| rest | | | | | | | |
| Monocytes | 0.0 | 0.0 | 0.0 | 0.0 | 0.0 | 0.0 | 0.0 |
| LPS | | | | | | | |
| Macrophages | 0.0 | 0.0 | 0.0 | 0.0 | 0.0 | 0.0 | 0.0 |
| rest | | | | | | | |
| Macrophages | 0.0 | 0.0 | 0.0 | 0.0 | 0.0 | 0.0 | 0.0 |
| LPS | | | | | | | |
| HUVEC | 0.0 | 0.0 | 0.0 | 0.0 | 0.0 | 0.0 | 0.0 |
| none | | | | | | | |
| HUVEC | 0.0 | 0.0 | 0.0 | 0.0 | 0.0 | 0.0 | 0.0 |
| starved | | | | | | | |
| HUVEC | 0.0 | 0.0 | 0.0 | 0.0 | 0.0 | 0.0 | 0.0 |
| IL-1beta | | | | | | | |
| HUVEC | 0.0 | 0.0 | 0.0 | 0.1 | 0.0 | 0.0 | 0.0 |
| IFN | | | | | | | |
| gamma | | | | | | | |
| HUVEC | 0.0 | 0.0 | 0.0 | 0.0 | 0.0 | 0.0 | 0.0 |
| TNF alpha + | | | | | | | |
| IFN | | | | | | | |
| gamma | | | | | | | |
| HUVEC | 0.0 | 0.0 | 0.0 | 0.0 | 0.0 | 0.0 | 0.0 |
| TNF alpha + | | | | | | | |
| IL4 | | | | | | | |
| HUVEC | 0.0 | 0.0 | 0.0 | 0.0 | 0.0 | 0.0 | 0.0 |
| IL-11 | | | | | | | |
| Lung | 0.0 | 0.0 | 0.0 | 0.0 | 0.0 | 0.0 | 0.0 |
| Microvascular | | | | | | | |
| EC | | | | | | | |
| none | | | | | | | |
| Lung | 0.0 | 7.3 | 0.0 | 0.0 | 0.0 | 0.0 | 0.0 |
| Microvascular | | | | | | | |
| EC | | | | | | | |
| TNF alpha + | | | | | | | |
| IL-1beta | | | | | | | |
| Microvascular | 0.0 | 0.0 | 0.0 | 0.0 | 0.0 | 0.0 | 0.0 |
| Dermal EC | | | | | | | |
| none | | | | | | | |
| Microsvascular | 0.0 | 0.0 | 0.0 | 0.0 | 0.0 | 0.0 | 0.0 |
| Dermal EC | | | | | | | |
| TNF alpha + | | | | | | | |
| IL-1beta | | | | | | | |
| Bronchial | 8.6 | 2.0 | 1.4 | 10.0 | 4.6 | 11.9 | 13.3 |
| epithelium | | | | | | | |
| TNF alpha + | | | | | | | |
| IL1beta | | | | | | | |
| Small | 21.8 | 9.5 | 17.8 | 17.7 | 17.8 | 12.2 | 17.2 |
| airway | | | | | | | |
| epithelium | | | | | | | |
| none | | | | | | | |
| Small | 100.0 | 100.0 | 100.0 | 100.0 | 100.0 | 100.0 | 100.0 |
| airway | | | | | | | |
| epithelium | | | | | | | |
| TNF alpha + | | | | | | | |
| IL-1beta | | | | | | | |
| Coronery | 0.2 | 0.2 | 0.3 | 0.4 | 0.4 | 0.2 | 0.2 |
| artery | | | | | | | |
| SMC rest | | | | | | | |
| Coronery | 0.2 | 0.3 | 0.2 | 0.2 | 0.2 | 0.1 | 0.2 |
| artery | | | | | | | |
| SMC | | | | | | | |
| TNF alpha + | | | | | | | |
| IL-1beta | | | | | | | |
| Astrocytes | 23.3 | 19.3 | 20.4 | 30.8 | 20.7 | 16.6 | 21.8 |
| rest | | | | | | | |
| Astrocytes | 27.0 | 25.0 | 25.3 | 27.9 | 22.7 | 19.3 | 28.5 |
| TNF alpha + | | | | | | | |
| IL-1beta | | | | | | | |
| KU-812 | 0.0 | 0.0 | 0.0 | 0.0 | 0.0 | 0.0 | 0.0 |
| (Basophil) | | | | | | | |
| rest | | | | | | | |
| KU-812 | 0.0 | 0.0 | 0.0 | 0.0 | 0.0 | 0.0 | 0.0 |
| (Basophil) | | | | | | | |
| PMA/ionomycin | | | | | | | |
| CCD1106 | 18.4 | 12.7 | 14.3 | 16.7 | 12.5 | 10.2 | 13.5 |
| (Keratinocytes) | | | | | | | |
| none | | | | | | | |
| CCD1106 | 4.2 | 1.4 | 2.0 | 7.1 | 3.5 | 5.1 | 8.4 |
| (Keratinocytes) | | | | | | | |
| TNF alpha + | | | | | | | |
| IL-1beta | | | | | | | |
| Liver | 0.3 | 0.2 | 0.2 | 0.3 | 0.3 | 0.2 | 0.4 |
| cirrhosis | | | | | | | |
| Lupus | 0.0 | 0.0 | 0.0 | 0.0 | 0.0 | 0.0 | 0.0 |
| kidney | | | | | | | |
| NCI-H292 | 0.4 | 0.2 | 0.4 | 0.3 | 0.6 | 0.1 | 0.2 |
| none | | | | | | | |
| NCI-H292 | 9.3 | 4.8 | 7.5 | 8.9 | 8.2 | 5.3 | 8.7 |
| IL-4 | | | | | | | |
| NCI-H292 | 0.6 | 0.3 | 0.6 | 0.7 | 0.7 | 0.5 | 0.4 |
| IL-9 | | | | | | | |
| NCI-H292 | 4.0 | 5.1 | 3.9 | 4.5 | 4.2 | 2.9 | 3.3 |
| IL-13 | | | | | | | |
| NCI-H292 | 0.2 | 0.2 | 0.1 | 0.3 | 0.2 | 0.2 | 0.2 |
| IFN | | | | | | | |
| gamma | | | | | | | |
| HPAEC | 0.0 | 0.0 | 0.0 | 0.0 | 0.0 | 0.0 | 0.1 |
| none | | | | | | | |
| HPAEC | 0.0 | 0.0 | 0.0 | 0.0 | 0.0 | 0.0 | 0.0 |
| TNF alpha + | | | | | | | |
| IL-1beta | | | | | | | |
| Lung | 0.1 | 0.3 | 0.3 | 0.3 | 0.3 | 0.1 | 0.2 |
| fibroblast | | | | | | | |
| none | | | | | | | |
| Lung | 0.0 | 0.0 | 0.1 | 0.0 | 0.0 | 0.1 | 0.1 |
| fibroblast | | | | | | | |
| TNF alpha + | | | | | | | |
| IL-1beta | | | | | | | |
| Lung | 0.3 | 0.1 | 0.2 | 0.3 | 0.1 | 0.2 | 0.3 |
| fibroblast | | | | | | | |
| IL-4 | | | | | | | |
| Lung | 0.0 | 0.2 | 0.1 | 0.1 | 0.1 | 0.1 | 0.1 |
| fibroblast | | | | | | | |
| IL-9 | | | | | | | |
| Lung | 0.1 | 0.1 | 0.1 | 0.2 | 0.2 | 0.0 | 0.3 |
| fibroblast | | | | | | | |
| IL-13 | | | | | | | |
| Lung | 0.1 | 0.0 | 0.0 | 0.1 | 0.1 | 0.2 | 0.1 |
| fibroblast | | | | | | | |
| IFN | | | | | | | |
| gamma | | | | | | | |
| Dermal | 2.2 | 1.3 | 2.4 | 3.1 | 2.5 | 1.5 | 2.4 |
| fibroblast | | | | | | | |
| CCD1070 | | | | | | | |
| rest | | | | | | | |
| Dermal | 1.0 | 0.8 | 1.1 | 2.1 | 1.8 | 0.9 | 1.2 |
| fibroblast | | | | | | | |
| CCD1070 | | | | | | | |
| TNF alpha | | | | | | | |
| Dermal | 0.3 | 0.3 | 0.6 | 0.7 | 0.6 | 0.4 | 0.6 |
| fibroblast | | | | | | | |
| CCD1070 | | | | | | | |
| IL-1beta | | | | | | | |
| Dermal | 0.6 | 0.6 | 1.1 | 1.5 | 1.1 | 0.6 | 0.7 |
| fibroblast | | | | | | | |
| IFN | | | | | | | |
| gamma | | | | | | | |
| Dermal | 1.8 | 1.2 | 2.0 | 2.5 | 1.5 | 1.2 | 1.4 |
| fibroblast | | | | | | | |
| IL-4 | | | | | | | |
| IBD | 0.1 | 0.1 | 0.0 | 0.0 | 0.0 | 0.0 | 0.0 |
| Colitis 2 | | | | | | | |
| IBD | 0.0 | 0.0 | 0.0 | 0.0 | 0.0 | 0.0 | 0.0 |
| Crohn's | | | | | | | |
| Colon | 0.0 | 0.0 | 0.1 | 0.2 | 0.2 | 0.1 | 0.2 |
| Lung | 0.3 | 0.3 | 0.2 | 0.6 | 0.3 | 0.2 | 0.2 |
| Thymus | 0.8 | 1.5 | 0.9 | 1.4 | 1.2 | 1.0 | 1.4 |
| Kidney | 3.0 | 2.8 | 3.6 | 4.5 | 3.3 | 2.0 | 2.6 |

CNS_neurodegeneration_v1.0 Summary: Ag047/Ag2679/Ag2728 This panel does not show differential expression of the NOV37 gene in Alzheimer's disease. However, this expression profile confirms the presence of this gene in the brain. Please see Panel 1 for discussion of utility of this gene in the central nervous system.

Panel 1 Summary: Ag047/Ag332/Ag47b Multiple experiments with three different probe and primer sets produce results that are in excellent agreement, with highest expression of the NOV37 gene in a brain cancer cell line (CT=23-25). There is also significant expression in clusters of samples from melanoma, ovarian cancer, breast, lung, renal colon and brain cancer lines. Thus, expression of this gene may be associated with these forms of cancer and could potentially be used as a diagnostic marker for the presence of these cancers. Furthermore, therapeutic modulation of the expression or function of these genes may be useful in the treatment of ovarian, breast, lung, renal, and brain cancer and melanoma.

In addition to significant expression in brain cancer cell lines, this gene is preferentially expressed in the brain. This expression profile suggests that this gene prodcut may play a role in CNS processes. This gene encodes a homolog of a member of the neurestin family, Ten M2, and may play a role in neuronal regeneration. Thus, agents that induce the expression or activity of NOV37 may have utility as neuronal regeneration drugs. Such agents would have utility in neurodegenerative diseases, stroke, and neuronal trauma.

Among tissues with metabolic function, this gene shows consistent expression in thyroid, adult and fetal heart, liver and skeletal muscle. Thus, this gene product may be an antibody target for the treatment of metabolic and endocrine disease, including obesity and Types 1 and 2 diabetes. In addition, this gene is expressed at higher levels in adult liver than in fetal liver and may be useful for differentiating between the two sources of liver tissue.

References:

Otaki J M, Firestein S. Neurestin: putative transmembrane molecule implicated in neuronal development. Dev Biol Aug. 1, 1999;212(1):165-81

We have cloned a novel cDNA encoding a putative transmembrane protein, neurestin, from the rat olfactory bulb. Neurestin was identified based on a sequence similar to that of the second extracellular loops of odorant receptors in the cysteine-rich CC box located immediately after EGF-like motifs. Neurestin shows homology to a neuregulin gene product, human gamma-heregulin, a Drosophila receptor-type pair-rule gene product, Odd Oz (Odz)/Ten(m), and Ten(a), suggesting a possible function in synapse formation and morphogenesis. Recently, a mouse neurestin homolog has independently been cloned as DOC4 from the NIH-3T3 cell line. Northern blot analysis showed that neurestin is highly expressed in the brain and also in other tissues at much lower levels. In situ hybridization studies showed that neurestin is expressed in many types of neurons, including pyramidal cells in the cerebral cortex and tufted cells in the olfactory bulb during development. In adults, neurestin is mainly expressed in olfactory and hippocampal granule cells, which are known to be generated throughout adulthood. Nonetheless, in adults the expression of neurestin was experimentally induced in external tufted cells during regeneration of olfactory sensory neurons. These results suggest a role for neurestin in neuronal development and regeneration in the central nervous system.

Panels 1.1/1.2/1.3D Summary: Ag047 The NOV37 gene is expressed in melanoma, ovarian cancer, breast, lung, renal and brain cancer lines with good concordance for 3 independent runs. Expression of this gene might be associated with these forms of cancer and therapeutic modulation of this gene might be of use in the treatment of these cancers. Please note that results from one experiment on Panel 1.3D with the probe/primer set Ag2975 are not included. The amp plot suggests that there were experimental difficulties with this run.

Panel 2.2 Summary: Ag2975 This gene appears to be expressed at a very low level in the samples used in this panel. Significant expression is only seen in lung, kidney and breast cancer samples. Expression of this gene might be associated with these forms of cancer and therapeutic modulation of this gene might be of use in the treatment of these cancers.

Panel 2D Summary: Ag047 The expression of the NOV37 gene was assessed in multiple runs on this panel, with excellent concordance between the runs. This gene appears to be expressed at a higher level in gastric, bladder, and 2 samples each of lung and kidney cancer when compared to the normal adjacent tissue. Thus, expression of this gene might be associated with these forms of cancer and therapeutic modulation of this gene might be of use in the treatment of these cancers.

Panel 3D Summary: Ag047 The NOV37 gene is expressed in squamous cell carcinoma, glioma, small cell lung cancer cell lines. Thus, expression of this gene might be associated with these cancers and therapeutic modulation of this gene might be of use in the treatment of these cancers.

Panels 4D/4.1D Summary: Ag047/Ag2679/Ag2728/Ag2975/Ag047b Multiple runs with different set of primers give very consistent expression data. Highest expression of the NOV37 transcript is found in small airway epithelium upon treatment with TNF-a and Il-1 (CT=25). This expression is significantly up-regulated when compared to untreated tissue (CT=28). Moderate expression of this transcript is also found in keratinocytes and astrocytes. NOV37 encodes for a neurestin like molecule whose role in neuronal regeneration has been demonstrated. Therefore, the putative protein encoded by NOV37 may play an important role in the regeneration or repair mechanism of these tissues in inflammation. Thus, therapeutic modulation of the expression of this gene product may be beneficial for the treatment of inflammatory lung diseases such as bronchitis, chronic obstructive pulmonary disease, emphysema. Therapeutics designed against this putative protein may also be useful for in the CNS for reducing inflammation, including inflammation that results from multiple sclerosis or stroke.

NOV38, NOV39a, and NOV39b

Expression of gene NOV38 and variants NOV39a and NOV39b was assessed using the primer-probe set Ag3753, described in Table AHA. Results of the RTQ-PCR runs are shown in Tables AHB and AHC.

TABLE AHA

Probe Name Ag3753

| | | | | |
|---|---|---|---|---|
| | | | Start | |
| Primers | Sequences | Length | Position | SEQ ID NO: |

| | | | | |
|---|---|---|---|---|
| Forward | 5′-cactgcagtaattcagctggta-3′ | 22 | 835 | 1135 |
| Probe | TET-5′-agtatccagtcccgccatcccagtt-3′- | 25 | 797 | 1136 |
| | TAMRA | | | |
| Reverse | 5′-aggcgagaccattacgtagact-3′ | 22 | 769 | 1137 |

TABLE AHB
General_screening_panel_v1.4

| | Rel. Exp. (%) Ag3753, | | Rel. Exp. (%) Ag3753, |
|---|---|---|---|
| Tissue Name | Run 216707728 | Tissue Name | Run 216707728 |
| Adipose | 0.0 | Renal ca. TK-10 | 1.0 |
| Melanoma* | 0.3 | Bladder | 0.0 |
| Hs688(A).T | | | |
| Melanoma* | 0.3 | Gastric ca. (liver met.) | 0.1 |
| Hs688(B).T | | NCI-N87 | |
| Melanoma* M14 | 0.0 | Gastric ca. KATO III | 0.0 |
| Melanoma* | 0.0 | Colon ca. SW-948 | 0.0 |
| LOXIMVI | | | |
| Melanoma* SK- | 0.0 | Colon ca. SW480 | 0.2 |
| MEL-5 | | | |
| Squamous cell | 0.2 | Colon ca.* (SW480 | 2.6 |
| carcinoma SCC-4 | | met) SW620 | |
| Testis Pool | 0.0 | Colon ca. HT29 | 0.0 |
| Prostate ca.* (bone | 0.1 | Colon ca. HCT-116 | 0.0 |
| met) PC-3 | | | |
| Prostate Pool | 0.0 | Colon ca. CaCo-2 | 0.0 |
| Placenta | 0.0 | Colon cancer tissue | 0.0 |
| Uterus Pool | 0.0 | Colon ca. SW1116 | 0.0 |
| Ovarian ca. | 0.1 | Colon ca. Colo-205 | 0.0 |
| OVCAR-3 | | | |
| Ovarian ca. SK-OV-3 | 0.0 | Colon ca. SW-48 | 0.0 |
| Ovarian ca. | 0.1 | Colon Pool | 0.0 |
| OVCAR-4 | | | |
| Ovarian ca. | 100.0 | Small Intestine Pool | 0.0 |
| OVCAR-5 | | | |
| Ovarian ca. IGROV-1 | 0.1 | Stomach Pool | 0.0 |
| Ovarian ca. | 2.1 | Bone Marrow Pool | 0.0 |
| OVCAR-8 | | | |
| Ovary | 0.0 | Fetal Heart | 0.0 |
| Breast ca. MCF-7 | 0.1 | Heart Pool | 0.9 |
| Breast ca. MDA- | 0.0 | Lymph Node Pool | 0.0 |
| MB-231 | | | |
| Breast ca. BT 549 | 0.0 | Fetal Skeletal Muscle | 0.0 |
| Breast ca. T47D | 0.5 | Skeletal Muscle Pool | 0.0 |
| Breast ca. MDA-N | 0.0 | Spleen Pool | 0.0 |
| Breast Pool | 0.0 | Thymus Pool | 0.0 |
| Trachea | 0.0 | CNS cancer (glio/astro) | 0.2 |
| | | U87-MG | |
| Lung | 0.0 | CNS cancer (glio/astro) | 0.2 |
| | | U-118-MG | |
| Fetal Lung | 0.0 | CNS cancer | 0.0 |
| | | (neuro; met) SK-N-AS | |
| Lung ca. NCI-N417 | 0.2 | CNS cancer (astro) SF- | 1.1 |
| | | 539 | |
| Lung ca. LX-1 | 1.9 | CNS cancer (astro) | 0.4 |
| | | SNB-75 | |
| Lung ca. NCI-H146 | 0.1 | CNS cancer (glio) | 0.2 |
| | | SNB-19 | |
| Lung ca. SHP-77 | 0.9 | CNS cancer (glio) SF- | 3.1 |
| | | 295 | |
| Lung ca. A549 | 0.1 | Brain (Amygdala) Pool | 0.0 |
| Lung ca. NCI-H526 | 0.0 | Brain (cerebellum) | 0.1 |
| Lung ca. NCI-H23 | 0.2 | Brain (fetal) | 0.0 |
| Lung ca. NCI-H460 | 0.1 | Brain (Hippocampus) | 0.0 |
| | | Pool | |
| Lung ca. HOP-62 | 0.0 | Cerebral Cortex Pool | 0.0 |
| Lung ca. NCI-H522 | 0.2 | Brain (Substantia nigra) | 0.0 |
| | | Pool | |
| Liver | 1.2 | Brain (Thalamus) Pool | 0.0 |
| Fetal Liver | 0.9 | Brain (whole) | 0.1 |
| Liver ca. HepG2 | 2.0 | Spinal Cord Pool | 0.0 |
| Kidney Pool | 0.0 | Adrenal Gland | 0.0 |
| Fetal Kidney | 0.0 | Pituitary gland Pool | 0.0 |
| Renal ca. 786-0 | 0.0 | Salivary Gland | 0.0 |
| Renal ca. A498 | 0.0 | Thyroid (female) | 0.0 |
| Renal ca. ACHN | 0.1 | Pancreatic ca. CAPAN2 | 0.0 |
| Renal ca. UO-31 | 0.0 | Pancreas Pool | 0.0 |

TABLE AHC
Panel 4.1D

| | Rel. Exp. (%) | | Rel. Exp. (%) |
|---|---|---|---|
| | Ag3753, Run | | Ag3753, Run |
| Tissue Name | 172209242 | Tissue Name | 172209242 |
| Secondary Th1 act | 0.5 | HUVEC IL-1beta | 0.5 |
| Secondary Th2 act | 2.1 | HUVEC IFN gamma | 1.0 |
| Secondary Tr1 act | 0.0 | HUVEC TNF alpha + IFN | 0.0 |
| | | gamma | |
| Secondary Th1 rest | 1.2 | HUVEC TNF alpha + IL4 | 0.0 |
| Secondary Th2 rest | 0.2 | HUVEC IL-11 | 0.0 |
| Secondary Tr1 rest | 1.0 | Lung Microvascular EC | 0.9 |
| | | none | |
| Primary Th1 act | 0.5 | Lung Microvascular EC | 0.0 |
| | | TNF alpha + IL-1beta | |
| Primary Th2 act | 0.0 | Microvascular Dermal EC | 0.0 |
| | | none | |
| Primary Tr1 act | 0.2 | Microsvasular Dermal EC | 0.0 |
| | | TNF alpha + IL-1beta | |
| Primary Th1 rest | 0.5 | Bronchial epithelium | 1.2 |
| | | TNF alpha + IL1beta | |
| Primary Th2 rest | 0.0 | Small airway epithelium | 0.0 |
| | | none | |
| Primary Tr1 rest | 0.5 | Small airway epithelium | 0.0 |
| | | TNF alpha + IL-1beta | |
| CD45RA CD4 | 1.7 | Coronery artery SMC rest | 2.6 |
| lymphocyte act | | | |
| CD45RO CD4 | 0.0 | Coronery artery SMC | 1.3 |
| lymphocyte act | | TNF alpha + IL-1beta | |
| CD8 lymphocyte act | 0.0 | Astrocytes rest | 1.2 |
| Secondary CD8 | 0.2 | Astrocytes TNF alpha + | 0.4 |
| lymphocyte rest | | IL-1beta | |
| Secondary CD8 | 3.6 | KU-812 (Basophil) rest | 0.0 |
| lymphocyte act | | | |
| CD4 lymphocyte none | 0.0 | KU-812 (Basophil) | 0.0 |
| | | PMA/ionomycin | |
| 2ry Th1/Th2/Tr1_anti- | 0.5 | CCD1106 (Keratinocytes) | 0.0 |
| CD95 CH11 | | none | |
| LAK cells rest | 2.5 | CCD1106 (Keratinocytes) | 0.0 |
| | | TNF alpha + IL-1beta | |
| LAK cells IL-2 | 0.0 | Liver cirrhosis | 39.5 |
| LAK cells IL-2 + IL-12 | 1.3 | NCI-H292 none | 0.3 |
| LAK cells IL-2 + IFN | 1.0 | NCI-H292 IL-4 | 1.1 |
| gamma | | | |
| LAK cells IL-2 + IL-18 | 0.5 | NCI-H292 IL-9 | 0.0 |
| LAK cells | 3.8 | NCI-H292 IL-13 | 0.8 |
| PMA/ionomycin | | | |
| NK Cells IL-2 rest | 0.3 | NCI-H292 IFN gamma | 0.3 |
| Two Way MLR 3 day | 0.5 | HPAEC none | 0.0 |
| Two Way MLR 5 day | 2.6 | HPAEC TNF alpha + IL- | 0.0 |
| | | 1beta | |
| Two Way MLR 7 day | 1.0 | Lung fibroblast none | 15.1 |
| PBMC rest | 0.0 | Lung fibroblast TNF alpha + | 1.2 |
| | | IL-1beta | |
| PBMC PWM | 0.0 | Lung fibroblast IL-4 | 0.8 |
| PBMC PHA-L | 0.5 | Lung fibroblast IL-9 | 0.0 |
| Ramos (B cell) none | 12.8 | Lung fibroblast IL-13 | 0.4 |
| Ramos (B cell) | 100.0 | Lung fibroblast IFN | 0.8 |
| ionomycin | | gamma | |
| B lymphocytes PWM | 0.5 | Dermal fibroblast | 3.7 |
| | | CCD1070 rest | |
| B lymphocytes CD40L | 0.9 | Dermal fibroblast | 2.3 |
| and IL-4 | | CCD1070 TNF alpha | |
| EOL-1 dbcAMP | 0.0 | Dermal fibroblast | 2.2 |
| | | CCD1070 IL-1beta | |
| EOL-1 dbcAMP | 0.0 | Dermal fibroblast IFN | 0.0 |
| PMA/ionomycin | | gamma | |
| Dendritic cells none | 0.9 | Dermal fibroblast IL-4 | 4.3 |
| Dendritic cells LPS | 6.2 | Dermal Fibroblasts rest | 0.9 |
| Dendritic cells anti- | 3.0 | Neutrophils TNFa + LPS | 0.0 |
| CD40 | | | |
| Monocytes rest | 0.0 | Neutrophils rest | 0.0 |
| Monocytes LPS | 0.5 | Colon | 0.8 |
| Macrophages rest | 3.7 | Lung | 0.0 |
| Macrophages LPS | 1.6 | Thymus | 1.1 |
| HUVEC none | 0.0 | Kidney | 15.3 |
| HUVEC starved | 0.0 | | |

General_screening_panel_v1.4 Summary: Ag3753 Results from one experiment with the NOV38 gene are not included. The amp plot indicates that there were experimental difficulties with this run.

Panel 4.1D Summary: Ag3753 Highest expression of the NOV38 transcript is found in Ramos B cell line activated with PMA and ionomycin (CT=29). However, expression is not seen in primary activated B cells. Therefore, epxression of this gene could potentially be used as a marker for activated B lymphoma. This gene is also expressed at lower levels in liver cirrhosis, lung fibroblasts and kidney. This transcript encodes for a molecule that belongs to the activin family, a member of the TGF beta superfamily. These factors influence growth and differentiation and are present in many cells and tissues. Therefore, therapeutics using the protein encoded by NOV38 could be important for the normal homeostasis of these tissues.

NOV40

Expression of gene NOV40 was assessed using the primer-probe set Ag2907, described in Table AIA. Results of the RTQ-PCR runs are shown in Tables AIB, AIC, AID and AIE.

TABLE AIA

Probe Name Ag2907

| | | | Start | |
|---|---|---|---|---|
| Primers | Sequences | Length | Position | SEQ ID NO: |
| Forward | 5′-ggctcattcgaaactactggta-3′ | 22 | 773 | 1138 |
| Probe | TET-5′-tggaatttcctcgcccactcttacct-3′- | 26 | 797 | 1139 |
| | TAMRA | | | |
| Reverse | 5′-gttgacaggtttgcagtagag-3′ | 22 | 844 | 1140 |

TABLE AIB
Panel 1.3D

| | Rel. Exp. (%) | Rel. Exp. (%) | | Rel. Exp. (%) | Rel. Exp. (%) |
|---|---|---|---|---|---|
| | Ag2907, Run | Ag2907, Run | | Ag2907, Run | Ag2907, Run |
| Tissue Name | 157283423 | 165701505 | Tissue Name | 157283423 | 165701505 |
| Liver | 0.0 | 0.0 | Kidney (fetal) | 0.0 | 0.0 |
| adenocarcinoma | | | | | |
| Pancreas | 0.0 | 0.0 | Renal ca. 786-0 | 0.0 | 0.0 |
| Pancreatic ca. | 0.0 | 0.0 | Renal ca. | 0.0 | 0.0 |
| CAPAN 2 | | | A498 | | |
| Adrenal gland | 0.0 | 0.0 | Renal ca. RXF | 0.0 | 0.0 |
| | | | 393 | | |
| Thyroid | 0.0 | 0.0 | Renal ca. | 0.0 | 0.0 |
| | | | ACHN | | |
| Salivary gland | 0.0 | 0.0 | Renal ca. UO- | 0.0 | 0.0 |
| | | | 31 | | |
| Pituitary gland | 100.0 | 100.0 | Renal ca. TK- | 2.0 | 28.3 |
| | | | 10 | | |
| Brain (fetal) | 0.0 | 0.0 | Liver | 0.0 | 0.0 |
| Brain (whole) | 0.0 | 14.3 | Liver (fetal) | 0.0 | 0.0 |
| Brain (amygdala) | 0.0 | 0.0 | Liver ca. | 0.0 | 0.0 |
| | | | (hepatoblast) | | |
| | | | HepG2 | | |
| Brain (cerebellum) | 0.0 | 11.9 | Lung | 0.0 | 0.0 |
| Brain | 1.9 | 0.0 | Lung (fetal) | 0.0 | 0.0 |
| (hippocampus) | | | | | |
| Brain (substantia | 0.0 | 0.0 | Lung ca. | 0.0 | 0.0 |
| nigra) | | | (small cell) | | |
| | | | LX-1 | | |
| Brain (thalamus) | 0.0 | 0.0 | Lung ca. | 1.1 | 0.0 |
| | | | (small cell) | | |
| | | | NCI-H69 | | |
| Cerebral Cortex | 2.0 | 0.0 | Lung ca. | 0.0 | 0.0 |
| | | | (s.cell var.) | | |
| | | | SHP-77 | | |
| Spinal cord | 1.3 | 0.0 | Lung ca. (large | 0.0 | 0.0 |
| | | | cell)NCI-H460 | | |
| glio/astro U87-MG | 0.0 | 12.2 | Lung ca. (non- | 0.0 | 0.0 |
| | | | sm. cell) A549 | | |
| glio/astro U-118- | 0.0 | 0.0 | Lung ca. (non- | 0.0 | 0.0 |
| MG | | | s.cell) NCI- | | |
| | | | H23 | | |
| astrocytoma | 0.0 | 0.0 | Lung ca. (non- | 0.0 | 0.0 |
| SW1783 | | | s.cell) HOP-62 | | |
| neuro*; met SK-N- | 1.0 | 0.0 | Lung ca. (non- | 0.0 | 0.0 |
| AS | | | s.cl) NCI- | | |
| | | | H522 | | |
| astrocytoma SF- | 1.4 | 0.0 | Lung ca. | 0.9 | 0.0 |
| 539 | | | (squam.) SW | | |
| | | | 900 | | |
| astrocytoma SNB- | 1.0 | 0.0 | Lung ca. | 0.0 | 0.0 |
| 75 | | | (squam.) NCI- | | |
| | | | H596 | | |
| glioma SNB-19 | 1.8 | 0.0 | Mammary | 0.0 | 0.0 |
| | | | gland | | |
| glioma U251 | 0.8 | 0.0 | Breast ca.* | 1.4 | 2.5 |
| | | | (pl.ef) MCF-7 | | |
| glioma SF-295 | 1.0 | 0.0 | Breast ca.* | 2.6 | 0.0 |
| | | | (pl.ef) MDA- | | |
| | | | MB-231 | | |
| Heart (fetal) | 0.0 | 0.0 | Breast ca.* | 0.0 | 0.0 |
| | | | (pl.ef) T47D | | |
| Heart | 0.0 | 0.0 | Breast ca. BT- | 3.3 | 0.0 |
| | | | 549 | | |
| Skeletal muscle | 1.0 | 0.0 | Breast ca. | 0.0 | 0.0 |
| (fetal) | | | MDA-N | | |
| Skeletal muscle | 0.0 | 0.0 | Ovary | 0.0 | 0.0 |
| Bone marrow | 0.0 | 0.0 | Ovarian ca. | 2.9 | 0.0 |
| | | | OVCAR-3 | | |
| Thymus | 0.0 | 0.0 | Ovarian ca. | 0.0 | 0.0 |
| | | | OVCAR-4 | | |
| Spleen | 0.9 | 0.0 | Ovarian ca. | 3.0 | 10.9 |
| | | | OVCAR-5 | | |
| Lymph node | 0.0 | 0.0 | Ovarian ca. | 0.0 | 0.0 |
| | | | OVCAR-8 | | |
| Colorectal | 0.0 | 0.0 | Ovarian ca. | 0.0 | 0.0 |
| | | | IGROV-1 | | |
| Stomach | 0.0 | 0.0 | Ovarian ca.* | 0.0 | 0.0 |
| | | | (ascites) SK- | | |
| | | | OV-3 | | |
| Small intestine | 0.0 | 0.0 | Uterus | 0.0 | 0.0 |
| Colon ca. SW480 | 0.0 | 0.0 | Placenta | 0.9 | 0.0 |
| Colon ca.* | 0.0 | 0.0 | Prostate | 0.0 | 0.0 |
| SW620(SW480 | | | | | |
| met) | | | | | |
| Colon ca. HT29 | 1.0 | 0.0 | Prostate ca.* | 1.3 | 0.0 |
| | | | (bone met)PC-3 | | |
| Colon ca. HCT- | 0.0 | 0.0 | Testis | 16.6 | 9.5 |
| 116 | | | | | |
| Colon ca. CaCo-2 | 1.0 | 0.0 | Melanoma | 0.0 | 0.0 |
| | | | Hs688(A).T | | |
| Colon ca. | 0.0 | 11.0 | Melanoma* | 0.0 | 0.0 |
| tissue(ODO3866) | | | (met) | | |
| | | | Hs688(B).T | | |
| Colon ca. HCC- | 2.6 | 0.0 | Melanoma | 0.0 | 0.0 |
| 2998 | | | UACC-62 | | |
| Gastric ca.* (liver | 1.2 | 13.3 | Melanoma | 0.0 | 0.0 |
| met) NCI-N87 | | | M14 | | |
| Bladder | 0.0 | 6.7 | Melanoma | 0.0 | 0.0 |
| | | | LOX IMVI | | |
| Trachea | 0.0 | 0.0 | Melanoma* | 0.0 | 0.0 |
| | | | (met) SK- | | |
| | | | MEL-5 | | |
| Kidney | 0.0 | 0.0 | Adipose | 1.5 | 0.0 |

TABLE AIC
Panel 2D

| | Rel. Exp. (%) | | Rel. Exp. (%) |
|---|---|---|---|
| | Ag2907, Run | | Ag2907, Run |
| Tissue Name | 157284121 | Tissue Name | 157284121 |
| Normal Colon | 4.5 | Kidney Margin | 0.0 |
| | | 8120608 | |
| CC Well to Mod Diff | 2.1 | Kidney Cancer | 0.0 |
| (ODO3866) | | 8120613 | |
| CC Margin (ODO3866) | 2.3 | Kidney Margin | 0.0 |
| | | 8120614 | |
| CC Gr.2 rectosigmoid | 1.7 | Kidney Cancer | 0.0 |
| (ODO3868) | | 9010320 | |
| CC Margin (ODO3868) | 0.0 | Kidney Margin | 4.8 |
| | | 9010321 | |
| CC Mod Diff (ODO3920) | 0.0 | Normal Uterus | 0.0 |
| CC Margin (ODO3920) | 0.0 | Uterus Cancer 064011 | 1.5 |
| CC Gr.2 ascend colon | 0.0 | Normal Thyroid | 0.0 |
| (ODO3921) | | | |
| CC Margin (ODO3921) | 6.8 | Thyroid Cancer | 0.0 |
| | | 064010 | |
| CC from Partial | 0.0 | Thyroid Cancer | 0.0 |
| Hepatectomy (ODO4309) | | A302152 | |
| Mets | | | |
| Liver Margin (ODO4309) | 0.0 | Thyroid Margin | 15.2 |
| | | A302153 | |
| Colon mets to lung | 0.0 | Normal Breast | 1.7 |
| (OD04451-01) | | | |
| Lung Margin (OD04451- | 0.0 | Breast Cancer | 0.0 |
| 02) | | (OD04566) | |
| Normal Prostate 6546-1 | 0.0 | Breast Cancer | 0.0 |
| | | (OD04590-01) | |
| Prostate Cancer | 0.0 | Breast Cancer Mets | 2.2 |
| (OD04410) | | (OD04590-03) | |
| Prostate Margin | 0.0 | Breast Cancer | 2.8 |
| (OD04410) | | Metastasis | |
| | | (OD04655-05) | |
| Prostate Cancer | 100.0 | Breast Cancer 064006 | 0.0 |
| (OD04720-01) | | | |
| Prostate Margin | 0.0 | Breast Cancer 1024 | 0.0 |
| (OD04720-02) | | | |
| Normal Lung 061010 | 7.2 | Breast Cancer | 2.4 |
| | | 9100266 | |
| Lung Met to Muscle | 0.6 | Breast Margin | 0.0 |
| (ODO4286) | | 9100265 | |
| Muscle Margin | 0.0 | Breast Cancer | 0.0 |
| (ODO4286) | | A209073 | |
| Lung Malignant Cancer | 3.4 | Breast Margin | 0.0 |
| (OD03126) | | A2090734 | |
| Lung Margin (OD03126) | 0.0 | Normal Liver | 0.0 |
| Lung Cancer (OD04404) | 0.0 | Liver Cancer 064003 | 3.5 |
| Lung Margin (OD04404) | 4.1 | Liver Cancer 1025 | 0.0 |
| Lung Cancer (OD04565) | 0.0 | Liver Cancer 1026 | 0.0 |
| Lung Margin (OD04565) | 0.0 | Liver Cancer 6004-T | 0.0 |
| Lung Cancer (OD04237- | 0.0 | Liver Tissue 6004-N | 0.5 |
| 01) | | | |
| Lung Margin (OD04237- | 0.0 | Liver Cancer 6005-T | 0.0 |
| 02) | | | |
| Ocular Mel Met to Liver | 0.0 | Liver Tissue 6005-N | 0.0 |
| (ODO4310) | | | |
| Liver Margin (ODO4310) | 0.0 | Normal Bladder | 0.0 |
| Melanoma Mets to Lung | 0.0 | Bladder Cancer 1023 | 0.0 |
| (OD04321) | | | |
| Lung Margin (OD04321) | 0.0 | Bladder Cancer | 22.1 |
| | | A302173 | |
| Normal Kidney | 0.0 | Bladder Cancer | 2.4 |
| | | (OD04718-01) | |
| Kidney Ca, Nuclear grade | 2.0 | Bladder Normal | 0.0 |
| 2 (OD04338) | | Adjacent (OD04718- | |
| | | 03) | |
| Kidney Margin | 0.0 | Normal Ovary | 0.0 |
| (OD04338) | | | |
| Kidney Ca Nuclear grade | 2.0 | Ovarian Cancer | 0.0 |
| 1/2 (OD04339) | | 064008 | |
| Kidney Margin | 0.0 | Ovarian Cancer | 14.4 |
| (OD04339) | | (OD04768-07) | |
| Kidney Ca, Clear cell | 0.0 | Ovary Margin | 0.0 |
| type (OD04340) | | (OD04768-08) | |
| Kidney Margin | 2.4 | Normal Stomach | 0.0 |
| (OD04340) | | | |
| Kidney Ca, Nuclear grade | 0.0 | Gastric Cancer | 0.0 |
| 3 (OD04348) | | 9060358 | |
| Kidney Margin | 0.0 | Stomach Margin | 0.0 |
| (OD04348) | | 9060359 | |
| Kidney Cancer | 0.0 | Gastric Cancer | 0.0 |
| (OD04622-01) | | 9060395 | |
| Kidney Margin | 0.0 | Stomach Margin | 0.0 |
| (OD04622-03) | | 9060394 | |
| Kidney Cancer | 0.0 | Gastric Cancer | 0.0 |
| (OD04450-01) | | 9060397 | |
| Kidney Margin | 0.0 | Stomach Margin 9060396 | 0.0 |
| (OD04450-03) | | | |
| Kidney Cancer 8120607 | 2.1 | Gastric Cancer | 2.9 |
| | | 064005 | |

TABLE AID
Panel 3D

| | Rel. Exp. (%) | | Rel. Exp. (%) |
|---|---|---|---|
| | Ag2907, Run | | Ag2907, Run |
| Tissue Name | 164633936 | Tissue Name | 164633936 |
| Daoy-Medulloblastoma | 0.0 | Ca Ski-Cervical epidermoid | 49.7 |
| | | carcinoma (metastasis) | |
| TE671-Medulloblastoma | 0.0 | ES-2-Ovarian clear cell | 0.0 |
| | | carcinoma | |
| D283 Med- | 0.0 | Ramos-Stimulated with | 0.0 |
| Medulloblastoma | | PMA/ionomycin 6 h | |
| PFSK-1-Primitive | 15.7 | Ramos-Stimulated with | 0.0 |
| Neuroectodermal | | PMA/ionomycin 14 h | |
| XF-498-CNS | 0.0 | MEG-01-Chronic | 0.0 |
| | | myelogenous leukemia | |
| | | (megokaryoblast) | |
| SNB-78-Glioma | 0.0 | Raji-Burkitt's lymphoma | 0.0 |
| SF-268-Glioblastoma | 0.0 | Daudi-Burkitt's lymphoma | 0.0 |
| T98G-Glioblastoma | 0.0 | U266-B-cell plasmacytoma | 0.0 |
| SK-N-SH- | 0.0 | CA46-Burkitt's lymphoma | 0.0 |
| Neuroblastoma | | | |
| (metastasis) | | | |
| SF-295-Glioblastoma | 0.0 | RL-non-Hodgkin's B-cell | 49.7 |
| | | lymphoma | |
| Cerebellum | 0.0 | JM1-pre-B-cell lymphoma | 0.0 |
| Cerebellum | 0.0 | Jurkat-T cell leukemia | 0.0 |
| NCI-H292- | 94.0 | TF-1-Erythroleukemia | 0.0 |
| Mucoepidermoid lung | | | |
| carcinoma | | | |
| DMS-114-Small cell | 0.0 | HUT 78-T-cell lymphoma | 0.0 |
| lung cancer | | | |
| DMS-79-Small cell lung | 0.0 | U937-Histiocytic lymphoma | 0.0 |
| cancer | | | |
| NCI-H146-Small cell | 0.0 | KU-812-Myelogenous | 0.0 |
| lung cancer | | leukemia | |
| NCI-H526-Small cell | 0.0 | 769-P-Clear cell renal | 27.2 |
| lung cancer | | carcinoma | |
| NCI-N417-Small cell | 0.0 | Caki-2-Clear cell renal | 0.0 |
| lung cancer | | carcinoma | |
| NCI-H82-Small cell | 0.0 | SW 839-Clear cell renal | 0.0 |
| lung cancer | | carcinoma | |
| NCI-H157-Squamous | 0.0 | G401-Wilms' tumor | 0.0 |
| cell lung cancer | | | |
| (metastasis) | | | |
| NCI-H1155-Large cell | 0.0 | Hs766T-Pancreatic | 0.0 |
| lung cancer | | carcinoma (LN metastasis) | |
| NCI-H1299-Large cell | 16.4 | CAPAN-1-Pancreatic | 0.0 |
| lung cancer | | adenocarcinoma (liver | |
| | | metastasis) | |
| NCI-H727-Lung | 0.0 | SU86.86-Pancreatic | 0.0 |
| carcinoid | | carcinoma (liver metastasis) | |
| NCI-UMC-11-Lung | 0.0 | BxPC-3-Pancreatic | 0.0 |
| carcinoid | | adenocarcinoma | |
| LX-1-Small cell lung | 0.0 | HPAC-Pancreatic | 0.0 |
| cancer | | adenocarcinoma | |
| Colo-205-Colon cancer | 0.0 | MIA PaCa-2-Pancreatic | 0.0 |
| | | carcinoma | |
| KM12-Colon cancer | 0.0 | CFPAC-1-Pancreatic ductal | 0.0 |
| | | adenocarcinoma | |
| KM20L2-Colon cancer | 0.0 | PANC-1-Pancreatic | 0.0 |
| | | epithelioid ductal carcinoma | |
| NCI-H716-Colon cancer | 0.0 | T24-Bladder carcinma | 0.0 |
| | | (transitional cell) | |
| SW-48-Colon | 0.0 | 5637-Bladder carcinoma | 0.0 |
| adenocarcinoma | | | |
| SW1116-Colon | 0.0 | HT-1197-Bladder carcinoma | 17.6 |
| adenocarcinoma | | | |
| LS 174T-Colon | 0.0 | UM-UC-3-Bladder carcinma | 0.0 |
| adenocarcinoma | | (transitional cell) | |
| SW-948-Colon | 0.0 | A204-Rhabdomyosarcoma | 0.0 |
| adenocarcinoma | | | |
| SW-480-Colon | 0.0 | HT-1080-Fibrosarcoma | 0.0 |
| adenocarcinoma | | | |
| NCI-SNU-5-Gastric | 0.0 | MG-63-Osteosarcoma | 0.0 |
| carcinoma | | | |
| KATO III-Gastric | 0.0 | SK-LMS-1-Leiomyosarcoma | 0.0 |
| carcinoma | | (vulva) | |
| NCI-SNU-16-Gastric | 0.0 | SJRH30-Rhabdomyosarcoma | 0.0 |
| carcinoma | | (met to bone marrow) | |
| NCI-SNU-1-Gastric | 0.0 | A431-Epidermoid carcinoma | 0.0 |
| carcinoma | | | |
| RF-1-Gastric | 0.0 | WM266-4-Melanoma | 0.0 |
| adenocarcinoma | | | |
| RF-48-Gastric | 0.0 | DU 145-Prostate carcinoma | 0.0 |
| adenocarcinoma | | (brain metastasis) | |
| MKN-45-Gastric | 0.0 | MDA-MB-468-Breast | 37.6 |
| carcinoma | | adenocarcinoma | |
| NCI-N87-Gastric | 0.0 | SCC-4-Squamous cell | 0.0 |
| carcinoma | | carcinoma of tongue | |
| OVCAR-5-Ovarian | 0.0 | SCC-9-Squamous cell | 0.0 |
| carcinoma | | carcinoma of tongue | |
| RL95-2-Uterine | 0.0 | SCC-15-Squamous cell | 0.0 |
| carcinoma | | carcinoma of tongue | |
| HelaS3-Cervical | 0.0 | CAL 27-Squamous cell | 100.0 |
| adenocarcinoma | | carcinoma of tongue | |

TABLE AIE
Panel 4D

| | Rel. Exp. (%) | | Rel. Exp. (%) |
|---|---|---|---|
| | Ag2907, Run | | Ag2907, Run |
| Tissue Name | 157284733 | Tissue Name | 157284733 |
| Secondary Th1 act | 0.0 | HUVEC IL-1beta | 0.0 |
| Secondary Th2 act | 0.0 | HUVEC IFN gamma | 0.0 |
| Secondary Tr1 act | 0.0 | HUVEC TNF alpha + IFN | 0.0 |
| | | gamma | |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC | 0.0 |
| | | none | |
| Primary Th1 act | 12.3 | Lung Microvascular EC | 10.4 |
| | | TNF alpha + IL-1beta | |
| Primary Th2 act | 0.0 | Microvascular Dermal EC | 11.2 |
| | | none | |
| Primary Tr1 act | 0.0 | Microsvasular Dermal EC | 0.0 |
| | | TNF alpha + IL-1beta | |
| Primary Th1 rest | 11.7 | Bronchial epithelium | 12.9 |
| | | TNF alpha + IL1beta | |
| Primary Th2 rest | 0.0 | Small airway epithelium | 24.0 |
| | | none | |
| Primary Tr1 rest | 0.0 | Small airway epithelium | 100.0 |
| | | TNF alpha + IL-1beta | |
| CD45RA CD4 | 0.0 | Coronery artery SMC rest | 0.0 |
| lymphocyte act | | | |
| CD45RO CD4 | 6.1 | Coronery artery SMC | 0.0 |
| lymphocyte act | | TNF alpha + IL-1beta | |
| CD8 lymphocyte act | 0.0 | Astrocytes rest | 0.0 |
| Secondary CD8 | 0.0 | Astrocytes TNF alpha + | 12.0 |
| lymphocyte rest | | IL-1beta | |
| Secondary CD8 | 0.0 | KU-812 (Basophil) rest | 0.0 |
| lymphocyte act | | | |
| CD4 lymphocyte none | 4.5 | KU-812 (Basophil) | 0.0 |
| | | PMA/ionomycin | |
| 2ry Th1/Th2/Tr1_anti- | 0.0 | CCD1106 (Keratinocytes) | 0.0 |
| CD95 CH11 | | none | |
| LAK cells rest | 0.0 | CCD1106 (Keratinocytes) | 0.0 |
| | | TNF alpha + IL-1beta | |
| LAK cells IL-2 | 13.4 | Liver cirrhosis | 35.1 |
| LAK cells IL-2 + IL-12 | 0.0 | Lupus kidney | 0.0 |
| LAK cells IL-2 + IFN | 0.0 | NCI-H292 none | 33.0 |
| gamma | | | |
| LAK cells IL-2 + IL-18 | 0.0 | NCI-H292 IL-4 | 36.6 |
| LAK cells | 0.0 | NCI-H292 IL-9 | 23.3 |
| PMA/ionomycin | | | |
| NK Cells IL-2 rest | 0.0 | NCI-H292 IL-13 | 21.2 |
| Two Way MLR 3 day | 0.0 | NCI-H292 IFN gamma | 11.6 |
| Two Way MLR 5 day | 0.0 | HPAEC none | 13.3 |
| Two Way MLR 7 day | 0.0 | HPAEC TNF alpha + IL- | 0.0 |
| | | 1beta | |
| PBMC rest | 0.0 | Lung fibroblast none | 0.0 |
| PBMC PWM | 11.7 | Lung fibroblast TNF alpha + | 0.0 |
| | | IL-1beta | |
| PBMC PHA-L | 0.0 | Lung fibroblast IL-4 | 0.0 |
| Ramos (B cell) none | 0.0 | Lung fibroblast IL-9 | 0.0 |
| Ramos (B cell) | 0.0 | Lung fibroblast IL-13 | 0.0 |
| ionomycin | | | |
| B lymphocytes PWM | 0.0 | Lung fibroblast IFN | 0.0 |
| | | gamma | |
| B lymphocytes CD40L | 0.0 | Dermal fibroblast | 0.0 |
| and IL-4 | | CCD1070 rest | |
| EOL-1 dbcAMP | 0.0 | Dermal fibroblast | 0.0 |
| | | CCD1070 TNF alpha | |
| EOL-1 dbcAMP | 0.0 | Dermal fibroblast | 0.0 |
| PMA/ionomycin | | CCD1070 IL-1beta | |
| Dendritic cells none | 0.0 | Dermal fibroblast IFN | 0.0 |
| | | gamma | |
| Dendritic cells LPS | 0.0 | Dermal fibroblast IL-4 | 0.0 |
| Dendritic cells anti- | 0.0 | IBD Colitis 2 | 13.1 |
| CD40 | | | |
| Monocytes rest | 0.0 | IBD Crohn's | 6.9 |
| Monocytes LPS | 26.1 | Colon | 0.0 |
| Macrophages rest | 0.0 | Lung | 0.0 |
| Macrophages LPS | 0.0 | Thymus | 16.0 |
| HUVEC none | 0.0 | Kidney | 13.1 |
| HUVEC starved | 0.0 | | |

Panel 1.3D Summary: Ag2907 Results from two experiments with the same probe/primer set are in good agreement. Expression of the NOV40 gene is highest in a sample derived from pituitary tissue with little to no expression detected in any other tissue. Thus, expression of this gene could be used to distinguish pituitary gland from the other samples on this panel.

The protein encoded for by this gene is most homologous to a glucuronosyltransferase, normally found in liver. UDP glycosyltransferases (UGT) are a superfamily of enzymes that catalyze the addition of the glycosyl group from a UTP-sugar to a small hydrophobic molecule. Glucuronosyltransferases are membrane-bound microsomal enzymes that catalyze the transfer of glucuronic acid to a wide variety of exogenous and endogenous lipophilic substrates. These enzymes are of major importance in the detoxification and subsequent elimination of xenobiotics such as drugs and carcinogens. The pituitary plays a major role in the physiology of many different systems in the body. Therefore, this gene may play an essential role in maintaining proper function of the pituitary gland and many of its secreted peptides. Furthermore, therapeutic modulation of the activity of this gene or its protein product using small molecule drugs may be useful for the treatment of diabetes and diabetes as well as growth, reproductive, and endocrine disorders.

Panel 2D Summary: Ag2907 Expression of the NOV40 gene is highest and almost exclusive to a sample derived from a prostate cancer (CT=31.7). Thus, the expression of this gene could be used to distinguish prostate cancer from the other samples in the panel. Moreover, therapeutic modulation of the activity of this gene or its protein product, through the use of small molecule drugs, protein therapeutics or antibodies, might be of benefit in the treatment of prostate cancer.

Panel 3D Summary: Ag2907 Expression of the NOV40 gene is highest in a sample derived from a squamous cell carcinoma cell line (CT=33.8). Thus, the expression of this gene could be used to distinguish this sample from the other samples in the panel.

Panel 4D Summary: Ag2907 Expression of the NOV40 gene is detected at a very low level in small airway epithelium treated with the inflammatory cytokines TNF-a and IL-1b (CT=34.2). Thus, expression of this gene may be a marker of inflammation in the lung.

NOV41a and NOV41b

Expression of gene NOV41a and variant NOV41b was assessed using the primer-probe sets Ag1361 and Ag2953, described in Tables AJA and AJB. Results of the RTQ-PCR runs are shown in Tables AJC, AJD and AJE.

TABLE AJA

Probe Name Ag1361

| | | | Start | |
|---|---|---|---|---|
| Primers | Sequences | Length | Position | SEQ ID NO: |
| Forward | 5′-ctggtcaggtacctggatgtta-3′ | 22 | 1438 | 1141 |
| Probe | TET-5′-tccatcaatgaagagcttcatattcg-3′- | 26 | 1480 | 1142 |
| | TAMRA | | | |
| Reverse | 5′-cagcctttaagtgatccatcag-3′ | 22 | 1507 | 1143 |

TABLE AJB

Probe Name Ag2953

| | | | Start | |
|---|---|---|---|---|
| Primers | Sequences | Length | Position | SEQ ID NO: |
| Forward | 5′-ctggtcaggtacctggatgtta-3′ | 22 | 1438 | 1144 |
| Probe | TET-5′-tccatcaatgaagagcttcatattcg-3′- | 26 | 1480 | 1145 |
| | TAMRA | | | |
| Reverse | 5′-cagcctttaagtgatccatcag-3′ | 22 | 1507 | 1146 |

TABLE AJC
Panel 1.3D

| | Rel. Exp. (%) Ag1361, | | Rel. Exp. (%) Ag1361, |
|---|---|---|---|
| Tissue Name | Run 152953143 | Tissue Name | Run 152953143 |
| Liver adenocarcinoma | 0.0 | Kidney (fetal) | 2.1 |
| Pancreas | 0.0 | Renal ca. 786-0 | 0.0 |
| Pancreatic ca. CAPAN 2 | 0.0 | Renal ca. A498 | 0.0 |
| Adrenal gland | 0.0 | Renal ca. RXF 393 | 0.0 |
| Thyroid | 0.0 | Renal ca. ACHN | 0.0 |
| Salivary gland | 1.6 | Renal ca. UO-31 | 0.0 |
| Pituitary gland | 0.1 | Renal ca. TK-10 | 0.0 |
| Brain (fetal) | 0.0 | Liver | 0.0 |
| Brain (whole) | 0.2 | Liver (fetal) | 0.0 |
| Brain (amygdala) | 0.7 | Liver ca. | 0.0 |
| | | (hepatoblast) HepG2 | |
| Brain (cerebellum) | 0.0 | Lung | 0.0 |
| Brain (hippocampus) | 1.0 | Lung (fetal) | 0.0 |
| Brain (substantia nigra) | 0.0 | Lung ca. (small cell) | 0.0 |
| | | LX-1 | |
| Brain (thalamus) | 0.0 | Lung ca. (small cell) | 0.0 |
| | | NCI-H69 | |
| Cerebral Cortex | 0.0 | Lung ca. (s.cell var.) | 0.0 |
| | | SHP-77 | |
| Spinal cord | 0.2 | Lung ca. (large | 0.0 |
| | | cell)NCI-H460 | |
| glio/astro U87-MG | 0.0 | Lung ca. (non-sm. | 0.0 |
| | | cell) A549 | |
| glio/astro U-118-MG | 0.0 | Lung ca. (non-s.cell) | 0.0 |
| | | NCI-H23 | |
| astrocytoma SW1783 | 0.0 | Lung ca. (non-s.cell) | 0.0 |
| | | HOP-62 | |
| neuro*; met SK-N-AS | 0.0 | Lung ca. (non-s.cl) | 0.1 |
| | | NCI-H522 | |
| astrocytoma SF-539 | 0.0 | Lung ca. (squam.) | 0.0 |
| | | SW 900 | |
| astrocytoma SNB-75 | 0.0 | Lung ca. (squam.) | 0.0 |
| | | NCI-H596 | |
| glioma SNB-19 | 0.0 | Mammary gland | 0.0 |
| glioma U251 | 0.0 | Breast ca.* (pl.ef) | 0.2 |
| | | MCF-7 | |
| glioma SF-295 | 0.0 | Breast ca.* (pl.ef) | 0.0 |
| | | MDA-MB-231 | |
| Heart (fetal) | 0.0 | Breast ca.* (pl.ef) | 0.0 |
| | | T47D | |
| Heart | 0.0 | Breast ca. BT-549 | 0.0 |
| Skeletal muscle (fetal) | 0.0 | Breast ca. MDA-N | 0.0 |
| Skeletal muscle | 0.0 | Ovary | 0.0 |
| Bone marrow | 0.0 | Ovarian ca. OVCAR-3 | 0.5 |
| Thymus | 0.4 | Ovarian ca. OVCAR-4 | 0.0 |
| Spleen | 0.0 | Ovarian ca. OVCAR-5 | 0.7 |
| Lymph node | 0.0 | Ovarian ca. OVCAR-8 | 0.0 |
| Colorectal | 0.0 | Ovarian ca. IGROV-1 | 0.0 |
| Stomach | 100.0 | Ovarian ca.* (ascites) | 0.0 |
| | | SK-OV-3 | |
| Small intestine | 0.0 | Uterus | 0.0 |
| Colon ca. SW480 | 0.0 | Placenta | 0.0 |
| Colon ca.* | 0.0 | Prostate | 0.0 |
| SW620(SW480 met) | | | |
| Colon ca. HT29 | 0.0 | Prostate ca.* (bone | 0.0 |
| | | met)PC-3 | |
| Colon ca. HCT-116 | 0.0 | Testis | 2.1 |
| Colon ca. CaCo-2 | 0.0 | Melanoma | 0.0 |
| | | Hs688(A).T | |
| Colon ca. | 0.4 | Melanoma* (met) | 0.0 |
| tissue(ODO3866) | | Hs688(B).T | |
| Colon ca. HCC-2998 | 0.0 | Melanoma UACC-62 | 0.0 |
| Gastric ca.* (liver met) | 0.0 | Melanoma M14 | 0.0 |
| NCI-N87 | | | |
| Bladder | 0.0 | Melanoma LOX | 0.0 |
| | | IMVI | |
| Trachea | 0.4 | Melanoma* (met) | 0.0 |
| | | SK-MEL-5 | |
| Kidney | 12.8 | Adipose | 0.0 |

TABLE AJD
Panel 2D

| | Rel. Exp. (%) | | Rel. Exp. (%) |
|---|---|---|---|
| | Ag1361, Run | | Ag1361, |
| Tissue Name | 152953321 | Tissue Name | Run 152953321 |
| Normal Colon | 0.2 | Kidney Margin | 2.1 |
| | | 8120608 | |
| CC Well to Mod Diff | 0.0 | Kidney Cancer | 0.5 |
| (ODO3866) | | 8120613 | |
| CC Margin (ODO3866) | 0.0 | Kidney Margin | 2.6 |
| | | 8120614 | |
| CC Gr.2 rectosigmoid | 1.3 | Kidney Cancer | 0.0 |
| (ODO3868) | | 9010320 | |
| CC Margin (ODO3868) | 0.0 | Kidney Margin | 2.9 |
| | | 9010321 | |
| CC Mod Diff (ODO3920) | 0.6 | Normal Uterus | 0.0 |
| CC Margin (ODO3920) | 0.0 | Uterus Cancer 064011 | 0.2 |
| CC Gr.2 ascend colon | 0.2 | Normal Thyroid | 0.0 |
| (ODO3921) | | | |
| CC Margin (ODO3921) | 0.0 | Thyroid Cancer | 0.0 |
| | | 064010 | |
| CC from Partial | 0.0 | Thyroid Cancer | 0.0 |
| Hepatectomy (ODO4309) | | A302152 | |
| Mets | | | |
| Liver Margin (ODO4309) | 0.0 | Thyroid Margin | 0.0 |
| | | A302153 | |
| Colon mets to lung | 0.0 | Normal Breast | 0.0 |
| (OD04451-01) | | | |
| Lung Margin (OD04451- | 0.0 | Breast Cancer | 0.0 |
| 02) | | (OD04566) | |
| Normal Prostate 6546-1 | 0.0 | Breast Cancer | 0.0 |
| | | (OD04590-01) | |
| Prostate Cancer | 0.0 | Breast Cancer Mets | 0.0 |
| (OD04410) | | (OD04590-03) | |
| Prostate Margin | 0.0 | Breast Cancer | 0.0 |
| (OD04410) | | Metastasis | |
| | | (OD04655-05) | |
| Prostate Cancer | 0.0 | Breast Cancer 064006 | 0.0 |
| (OD04720-01) | | | |
| Prostate Margin | 0.1 | Breast Cancer 1024 | 0.3 |
| (OD04720-02) | | | |
| Normal Lung 061010 | 0.3 | Breast Cancer | 0.0 |
| | | 9100266 | |
| Lung Met to Muscle | 0.0 | Breast Margin | 0.0 |
| (ODO4286) | | 9100265 | |
| Muscle Margin | 0.0 | Breast Cancer | 0.1 |
| (ODO4286) | | A209073 | |
| Lung Malignant Cancer | 0.0 | Breast Margin | 0.1 |
| (OD03126) | | A2090734 | |
| Lung Margin (OD03126) | 0.0 | Normal Liver | 0.0 |
| Lung Cancer (OD04404) | 0.1 | Liver Cancer 064003 | 0.0 |
| Lung Margin (OD04404) | 0.1 | Liver Cancer 1025 | 0.0 |
| Lung Cancer (OD04565) | 0.0 | Liver Cancer 1026 | 0.2 |
| Lung Margin (OD04565) | 0.0 | Liver Cancer 6004-T | 0.0 |
| Lung Cancer (OD04237- | 0.0 | Liver Tissue 6004-N | 0.0 |
| 01) | | | |
| Lung Margin (OD04237- | 0.0 | Liver Cancer 6005-T | 0.0 |
| 02) | | | |
| Ocular Mel Met to Liver | 0.0 | Liver Tissue 6005-N | 0.0 |
| (ODO4310) | | | |
| Liver Margin (ODO4310) | 0.0 | Normal Bladder | 0.4 |
| Melanoma Mets to Lung | 0.0 | Bladder Cancer 1023 | 0.1 |
| (OD04321) | | | |
| Lung Margin (OD04321) | 0.0 | Bladder Cancer | 0.1 |
| | | A302173 | |
| Normal Kidney | 23.3 | Bladder Cancer | 0.0 |
| | | (OD04718-01) | |
| Kidney Ca, Nuclear grade | 0.7 | Bladder Normal | 0.0 |
| 2 (OD04338) | | Adjacent (OD04718- | |
| | | 03) | |
| Kidney Margin | 9.2 | Normal Ovary | 0.0 |
| (OD04338) | | | |
| Kidney Ca Nuclear grade | 0.0 | Ovarian Cancer | 0.2 |
| 1/2 (OD04339) | | 064008 | |
| Kidney Margin | 28.3 | Ovarian Cancer | 0.0 |
| (OD04339) | | (OD04768-07) | |
| Kidney Ca, Clear cell | 0.0 | Ovary Margin | 0.0 |
| type (OD04340) | | (OD04768-08) | |
| Kidney Margin | 45.4 | Normal Stomach | 100.0 |
| (OD04340) | | | |
| Kidney Ca, Nuclear grade | 0.0 | Gastric Cancer | 5.3 |
| 3 (OD04348) | | 9060358 | |
| Kidney Margin | 18.6 | Stomach Margin | 78.5 |
| (OD04348) | | 9060359 | |
| Kidney Cancer | 0.0 | Gastric Cancer | 0.3 |
| (OD04622-01) | | 9060395 | |
| Kidney Margin | 3.4 | Stomach Margin | 31.6 |
| (OD04622-03) | | 9060394 | |
| Kidney Cancer | 0.0 | Gastric Cancer | 0.2 |
| (OD04450-01) | | 9060397 | |
| Kidney Margin | 25.7 | Stomach Margin | 29.5 |
| (OD04450-03) | | 9060396 | |
| Kidney Cancer 8120607 | 0.0 | Gastric Cancer | 4.5 |
| | | 064005 | |

TABLE AJE
Panel 4D

| | Rel. | Rel. | | Rel. | Rel. |
|---|---|---|---|---|---|
| | Exp. (%) | Exp. (%) | | Exp. (%) | Exp. (%) |
| | Ag1361, | Ag2953, | | Ag1361, | Ag2953, |
| | Run | Run | | Run | Run |
| Tissue Name | 152953376 | 164306345 | Tissue Name | 152953376 | 164306345 |
| Secondary Th1 act | 0.0 | 0.0 | HUVEC IL-1beta | 0.0 | 0.0 |
| Secondary Th2 act | 0.0 | 0.0 | HUVEC IFN | 0.0 | 0.0 |
| | | | gamma | | |
| Secondary Tr1 act | 0.0 | 0.0 | HUVEC TNF | 0.0 | 0.0 |
| | | | alpha + IFN | | |
| | | | gamma | | |
| Secondary Th1 rest | 0.0 | 0.0 | HUVEC TNF | 0.0 | 0.0 |
| | | | alpha + IL4 | | |
| Secondary Th2 rest | 0.0 | 0.0 | HUVEC IL-11 | 0.0 | 0.0 |
| Secondary Tr1 rest | 0.0 | 0.0 | Lung | 0.0 | 0.0 |
| | | | Microvascular EC | | |
| | | | none | | |
| Primary Th1 act | 0.0 | 0.0 | Lung | 0.0 | 0.0 |
| | | | Microvascular EC | | |
| | | | TNF alpha + IL- | | |
| | | | 1beta | | |
| Primary Th2 act | 0.0 | 0.0 | Microvascular | 0.0 | 0.0 |
| | | | Dermal EC none | | |
| Primary Tr1 act | 0.0 | 0.0 | Microsvasular | 0.0 | 0.0 |
| | | | Dermal EC | | |
| | | | TNF alpha + IL- | | |
| | | | 1beta | | |
| Primary Th1 rest | 0.0 | 0.0 | Bronchial | 0.0 | 0.0 |
| | | | epithelium | | |
| | | | TNF alpha + | | |
| | | | IL1beta | | |
| Primary Th2 rest | 0.0 | 0.0 | Small airway | 0.0 | 0.0 |
| | | | epithelium none | | |
| Primary Tr1 rest | 0.0 | 0.0 | Small airway | 0.0 | 0.0 |
| | | | epithelium | | |
| | | | TNF alpha + IL- | | |
| | | | 1beta | | |
| CD45RA CD4 | 0.0 | 0.0 | Coronery artery | 0.0 | 0.0 |
| lymphocyte act | | | SMC rest | | |
| CD45RO CD4 | 0.0 | 0.0 | Coronery artery | 0.0 | 0.0 |
| lymphocyte act | | | SMC TNF alpha + | | |
| | | | IL-1beta | | |
| CD8 lymphocyte | 0.0 | 0.0 | Astrocytes rest | 0.0 | 0.0 |
| act | | | | | |
| Secondary CD8 | 0.0 | 0.0 | Astrocytes | 0.1 | 0.2 |
| lymphocyte rest | | | TNF alpha + IL- | | |
| | | | 1beta | | |
| Secondary CD8 | 0.0 | 0.0 | KU-812 | 0.0 | 0.0 |
| lymphocyte act | | | (Basophil) rest | | |
| CD4 lymphocyte | 0.0 | 0.0 | KU-812 | 0.0 | 0.0 |
| none | | | (Basophil) | | |
| | | | PMA/ionomycin | | |
| 2ry | 0.0 | 0.0 | CCD1106 | 0.0 | 0.0 |
| Th1/Th2/Tr1_anti- | | | (Keratinocytes) | | |
| CD95 CH11 | | | none | | |
| LAK cells rest | 0.0 | 0.0 | CCD1106 | 0.0 | 0.0 |
| | | | (Keratinocytes) | | |
| | | | TNF alpha + IL- | | |
| | | | 1beta | | |
| LAK cells IL-2 | 0.0 | 0.0 | Liver cirrhosis | 0.0 | 0.0 |
| LAK cells IL-2 + IL- | 0.0 | 0.0 | Lupus kidney | 1.2 | 0.8 |
| 12 | | | | | |
| LAK cells IL- | 0.0 | 0.0 | NCI-H292 none | 0.2 | 0.0 |
| 2 + IFN gamma | | | | | |
| LAK cells IL-2 + | 0.0 | 0.0 | NCI-H292 IL-4 | 0.0 | 0.0 |
| IL-18 | | | | | |
| LAK cells | 0.0 | 0.0 | NCI-H292 IL-9 | 0.2 | 0.0 |
| PMA/ionomycin | | | | | |
| NK Cells IL-2 rest | 0.0 | 0.0 | NCI-H292 IL-13 | 0.0 | 0.0 |
| Two Way MLR 3 | 0.0 | 0.0 | NCI-H292 IFN | 0.0 | 0.0 |
| day | | | gamma | | |
| Two Way MLR 5 | 0.0 | 0.0 | HPAEC none | 0.0 | 0.0 |
| day | | | | | |
| Two Way MLR 7 | 0.0 | 0.0 | HPAEC TNF | 0.0 | 0.0 |
| day | | | alpha + IL-1beta | | |
| PBMC rest | 0.0 | 0.0 | Lung fibroblast | 0.0 | 0.0 |
| | | | none | | |
| PBMC PWM | 0.0 | 0.2 | Lung fibroblast | 0.0 | 0.0 |
| | | | TNF alpha + IL-1 | | |
| | | | beta | | |
| PBMC PHA-L | 0.0 | 0.0 | Lung fibroblast | 0.0 | 0.0 |
| | | | IL-4 | | |
| Ramos (B cell) | 0.0 | 0.0 | Lung fibroblast | 0.0 | 0.0 |
| none | | | IL-9 | | |
| Ramos (B cell) | 0.0 | 0.0 | Lung fibroblast | 0.0 | 0.0 |
| ionomycin | | | IL-13 | | |
| B lymphocytes | 0.0 | 0.0 | Lung fibroblast | 0.0 | 0.0 |
| PWM | | | IFN gamma | | |
| B lymphocytes | 0.0 | 0.0 | Dermal fibroblast | 0.0 | 0.0 |
| CD40L and IL-4 | | | CCD1070 rest | | |
| EOL-1 dbcAMP | 0.0 | 0.0 | Dermal fibroblast | 0.0 | 0.0 |
| | | | CCD1070 TNF | | |
| | | | alpha | | |
| EOL-1 dbcAMP | 0.0 | 0.0 | Dermal fibroblast | 0.0 | 0.0 |
| PMA/ionomycin | | | CCD1070 IL-1 | | |
| | | | beta | | |
| Dendritic cells | 0.0 | 0.0 | Dermal fibroblast | 0.0 | 0.0 |
| none | | | IFN gamma | | |
| Dendritic cells LPS | 0.0 | 0.0 | Dermal fibroblast | 0.0 | 0.0 |
| | | | IL-4 | | |
| Dendritic cells anti- | 0.0 | 0.0 | IBD Colitis 2 | 0.0 | 0.0 |
| CD40 | | | | | |
| Monocytes rest | 0.0 | 0.0 | IBD Crohn's | 0.0 | 0.0 |
| Monocytes LPS | 0.0 | 0.0 | Colon | 0.0 | 0.0 |
| Macrophages rest | 0.0 | 0.0 | Lung | 0.0 | 0.0 |
| Macrophages LPS | 0.0 | 0.0 | Thymus | 100.0 | 100.0 |
| HUVEC none | 0.0 | 0.0 | Kidney | 0.0 | 0.2 |
| HUVEC starved | 0.0 | 0.0 | | | |

Panel 1.3D Summary: Ag1361 Expression of the NOV41a gene is restricted to stomach (CT value=29.9) and kidney (CT value=32.9) tissue. This observation is consistent with the identification of this gene as a sodium/hydrogen ion exchanger because the function of both of these tissues requires sodium/hydrogen ion exchange activity. The inhibition of the NOV41A protein activity, through the use of antibodies or small molecule drugs, might be of use in the treatment of kidney or gastric diseases related to the function of a sodium/hydrogen ion exchanger. For example, the activity of this gene may be related to over-production of stomach acid leading to acid reflux disease or peptic ulcer. Results from a second experiment with the probe and primer set Ag2953 are not included. The amp plot indicates that there is a potential problem in one of the sample wells.

Panel 2D Summary: Ag1361 Consistent with what was observed in Panel 1.3D, expression of the NOV41a gene in panel 2D is restricted to both normal kidney and stomach adjacent to tumor tissue. Interestingly, expression of the gene is absent in 4/4 gastric tumors and 10/10 kidney cancers when compared to the normal adjacent tissue controls. Thus, the expression of this gene appears to be a consistent trait of the non-neoplastic kidney and stomach. Therefore the absence of expression of this gene could be used as a diagnostic marker for kidney or gastric cancer. In addition, the replacement of this gene, potentially through the direct application of the protein or using gene replacement therapy, could be of use in the treatment of kidney or gastric cancer. Na+/H+exchangers have previously been implicated in modulation of cellular adhesion and tumor invasion (Refs. 1 and 2).

References:

1. Denker S. P., Huang D. C., Orlowski J., Furthmayr H., Barber D. L. (2000) Direct binding of the Na—H exchanger NHE1 to ERM proteins regulates the cortical cytoskeleton and cell shape independently of H(+) translocation. Mol. Cell 6: 1425-1436.

The association of actin filaments with the plasma membrane maintains cell shape and adhesion. Here, we show that the plasma membrane ion exchanger NHE1 acts as an anchor for actin filaments to control the integrity of the cortical cytoskeleton. This occurs through a previously unrecognized structural link between NHE1 and the actin binding proteins ezrin, radixin, and moesin (ERM). NHE1 and ERM proteins associate directly and colocalize in lamellipodia. Fibroblasts expressing NHE1 with mutations that disrupt ERM binding, but not ion translocation, have impaired organization of focal adhesions and actin stress fibers, and an irregular cell shape. We propose a structural role for NHE1 in regulating the cortical cytoskeleton that is independent of its function as an ion exchanger.

PMID: 11163215

2. Reshkin S. J., Bellizzi A., Albarani V., Guerra L., Tommasino M., Paradiso A., Casavola V. (2000) Phosphoinositide 3-kinase is involved in the tumor-specific activation of human breast cancer cell Na(+)/H(+) exchange, motility, and invasion induced by serum deprivation. J. Biol. Chem. 275: 5361-5369.

Whereas the tumor acidic extracellular pH plays a crucial role in the invasive process, the mechanism(s) behind this acidification, especially in low nutrient conditions, are unclear. The regulation of the Na(+)/H(+) exchanger (NHE) and invasion by serum deprivation were studied in a series of breast epithelial cell lines representing progression from non-tumor to highly metastatic cells. Whereas serum deprivation reduced lactate production in all three cells lines, it inhibited NHE activity in the non-tumor cells and stimulated it in the tumor cells with a larger stimulation in the metastatic cells. The stimulation of NHE in the tumor cell lines was the result of an increased affinity of the internal H(+) regulatory site of the NHE without changes in sodium kinetics or expression. Serum deprivation conferred increased cell motility and invasive ability that were abrogated by specific inhibition of the NHE. Inhibition of phosphoinositide 3-kinase by overexpression of a dominant-negative mutant or wortmannin incubation inhibited NHE activity and invasion in serum replete conditions while potentiating the serum deprivation-dependent activation of the NHE and invasion. These results indicate that the up-regulation of the NHE by a phosphoinositide 3-kinase-dependent mechanism plays an essential role in increased tumor cell invasion induced by serum deprivation.

PMID: 10681510

Panel 4D Summary: Ag1361/Ag2953 Two experiments with the same probe and primer sets produce results that are in excellent agreement. The NOV41a transcript is expressed in the thymus in Panel 4D (CT=28.6), but not in Panel 1.3D (CT=38). The NOV41A gene encodes a putative ion exchange molecule and may therefore be important in signal transduction in the thymus. Antibodies against the protein encoded for by the NOV41A gene may be used to identify thymic tissue. Additionally, small molecule or antibody therapeutics designed against this putative ion exchanger could disrupt T cell development in the thymus and serve an immunosuppresive function that could be important for tissue transplant.

NOV42a and NOV42b

Expression of gene NOV42a and variant NOV42b was assessed using the primer-probe set Ag3002, described in Table AKA. Results of the RTQ-PCR runs are shown in Tables AKB and AKC.

TABLE AKA
Probe Name Ag3002

| | | | Start | |
|---|---|---|---|---|
| Primers | Sequences | Length | Position | SEQ ID NO: |
| Forward | 5′-agaccctccatgtggtcatt-3′ | 20 | 1188 | 1147 |
| Probe | TET-5′-tcacaggaacagctacaaagaaccca-3′-TAMRA | 26 | 1211 | 1148 |
| Reverse | 5′-caggaccatctggagaagct-3′ | 20 | 1245 | 1149 |

TABLE AKB
Panel 1.3D

| | Rel. Exp. (%) Ag3002, | | Rel. Exp. (%) Ag3002, |
|---|---|---|---|
| Tissue Name | Run 167905704 | Tissue Name | Run 167905704 |
| Liver adenocarcinoma | 0.0 | Kidney (fetal) | 0.0 |
| Pancreas | 0.0 | Renal ca. 786-0 | 0.0 |
| Pancreatic ca. CAPAN 2 | 0.0 | Renal ca. A498 | 0.0 |
| Adrenal gland | 4.8 | Renal ca. RXF 393 | 0.0 |
| Thyroid | 0.0 | Renal ca. ACHN | 0.0 |
| Salivary gland | 3.0 | Renal ca. UO-31 | 0.0 |
| Pituitary gland | 0.0 | Renal ca. TK-10 | 0.0 |
| Brain (fetal) | 0.0 | Liver | 4.3 |
| Brain (whole) | 0.0 | Liver (fetal) | 0.0 |
| Brain (amygdala) | 0.0 | Liver ca. | 0.0 |
| | | (hepatoblast) HepG2 | |
| Brain (cerebellum) | 14.1 | Lung | 0.0 |
| Brain (hippocampus) | 6.7 | Lung (fetal) | 4.0 |
| Brain (substantia nigra) | 0.0 | Lung ca. (small cell) | 0.0 |
| | | LX-1 | |
| Brain (thalamus) | 0.0 | Lung ca. (small cell) | 0.0 |
| | | NCI-H69 | |
| Cerebral Cortex | 0.0 | Lung ca. (s.cell var.) | 0.0 |
| | | SHP-77 | |
| Spinal cord | 0.0 | Lung ca. (large | 0.0 |
| | | cell)NCI-H460 | |
| glio/astro U87-MG | 0.0 | Lung ca. (non-sm. | 0.0 |
| | | cell) A549 | |
| glio/astro U-118-MG | 0.0 | Lung ca. (non-s.cell) | 0.0 |
| | | NCI-H23 | |
| astrocytoma SW1783 | 4.0 | Lung ca. (non-s.cell) | 0.0 |
| | | HOP-62 | |
| neuro*; met SK-N-AS | 0.0 | Lung ca. (non-s.cl) | 3.2 |
| | | NCI-H522 | |
| astrocytoma SF-539 | 0.0 | Lung ca. (squam.) | 0.0 |
| | | SW 900 | |
| astrocytoma SNB-75 | 0.0 | Lung ca. (squam.) | 0.0 |
| | | NCI-H596 | |
| glioma SNB-19 | 0.0 | Mammary gland | 100.0 |
| glioma U251 | 0.0 | Breast ca.* (pl.ef) | 0.0 |
| | | MCF-7 | |
| glioma SF-295 | 0.0 | Breast ca.* (pl.ef) | 0.0 |
| | | MDA-MB-231 | |
| Heart (fetal) | 0.0 | Breast ca.* (pl.ef) | 29.9 |
| | | T47D | |
| Heart | 15.7 | Breast ca. BT-549 | 0.0 |
| Skeletal muscle (fetal) | 6.5 | Breast ca. MDA-N | 0.0 |
| Skeletal muscle | 20.6 | Ovary | 12.5 |
| Bone marrow | 0.0 | Ovarian ca. OVCAR-3 | 0.0 |
| Thymus | 13.7 | Ovarian ca. OVCAR-4 | 0.0 |
| Spleen | 71.7 | Ovarian ca. OVCAR-5 | 21.6 |
| Lymph node | 24.8 | Ovarian ca. OVCAR-8 | 15.2 |
| Colorectal | 3.8 | Ovarian ca. IGROV-1 | 0.0 |
| Stomach | 0.0 | Ovarian ca.* (ascites) | 0.0 |
| | | SK-OV-3 | |
| Small intestine | 33.2 | Uterus | 27.0 |
| Colon ca. SW480 | 0.0 | Placenta | 0.0 |
| Colon ca.* | 4.2 | Prostate | 0.0 |
| SW620(SW480 met) | | | |
| Colon ca. HT29 | 0.0 | Prostate ca.* (bone | 0.0 |
| | | met)PC-3 | |
| Colon ca. HCT-116 | 2.4 | Testis | 7.7 |
| Colon ca. CaCo-2 | 4.0 | Melanoma | 0.0 |
| | | Hs688(A).T | |
| Colon ca. | 0.0 | Melanoma* (met) | 0.0 |
| tissue(ODO3866) | | Hs688(B).T | |
| Colon ca. HCC-2998 | 0.0 | Melanoma UACC-62 | 0.0 |
| Gastric ca.* (liver met) | 0.0 | Melanoma M14 | 0.0 |
| NCI-N87 | | | |
| Bladder | 0.0 | Melanoma LOX | 0.0 |
| | | IMVI | |
| Trachea | 0.0 | Melanoma* (met) | 0.0 |
| | | SK-MEL-5 | |
| Kidney | 0.0 | Adipose | 13.8 |

TABLE AKC
Panel 4D

| | Rel. Exp. (%) | | Rel. Exp. (%) |
|---|---|---|---|
| | Ag3002, Run | | Ag3002, Run |
| Tissue Name | 164043126 | Tissue Name | 164043126 |
| Secondary Th1 act | 10.2 | HUVEC IL-1beta | 0.0 |
| Secondary Th2 act | 0.0 | HUVEC IFN gamma | 0.0 |
| Secondary Tr1 act | 0.0 | HUVEC TNF alpha + IFN | 0.0 |
| | | gamma | |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC | 0.0 |
| | | none | |
| Primary Th1 act | 0.0 | Lung Microvascular EC | 0.0 |
| | | TNF alpha + IL-1beta | |
| Primary Th2 act | 0.0 | Microvascular Dermal EC | 0.0 |
| | | none | |
| Primary Tr1 act | 0.0 | Microsvasular Dermal EC | 0.0 |
| | | TNF alpha + IL-1beta | |
| Primary Th1 rest | 14.2 | Bronchial epithelium | 0.0 |
| | | TNF alpha + IL1beta | |
| Primary Th2 rest | 0.0 | Small airway epithelium | 0.0 |
| | | none | |
| Primary Tr1 rest | 0.0 | Small airway epithelium | 0.0 |
| | | TNF alpha + IL-1beta | |
| CD45RA CD4 | 0.0 | Coronery artery SMC rest | 0.0 |
| lymphocyte act | | | |
| CD45RO CD4 | 6.2 | Coronery artery SMC | 0.0 |
| lymphocyte act | | TNF alpha + IL-1beta | |
| CD8 lymphocyte act | 0.0 | Astrocytes rest | 0.0 |
| Secondary CD8 | 0.0 | Astrocytes TNF alpha + | 0.0 |
| lymphocyte rest | | IL-1beta | |
| Secondary CD8 | 0.0 | KU-812 (Basophil) rest | 0.0 |
| lymphocyte act | | | |
| CD4 lymphocyte none | 0.0 | KU-812 (Basophil) | 0.0 |
| | | PMA/ionomycin | |
| 2ry Th1/Th2/Tr1_anti- | 0.0 | CCD1106 (Keratinocytes) | 0.0 |
| CD95 CH11 | | none | |
| LAK cells rest | 0.0 | CCD1106 (Keratinocytes) | 0.0 |
| | | TNF alpha + IL-1beta | |
| LAK cells IL-2 | 0.0 | Liver cirrhosis | 0.0 |
| LAK cells IL-2 + IL-12 | 0.0 | Lupus kidney | 10.8 |
| LAK cells IL-2 + IFN | 23.3 | NCI-H292 none | 10.8 |
| gamma | | | |
| LAK cells IL-2 + IL-18 | 0.0 | NCI-H292 IL-4 | 0.0 |
| LAK cells | 0.0 | NCI-H292 IL-9 | 0.0 |
| PMA/ionomycin | | | |
| NK Cells IL-2 rest | 0.0 | NCI-H292 IL-13 | 0.0 |
| Two Way MLR 3 day | 0.0 | NCI-H292 IFN gamma | 0.0 |
| Two Way MLR 5 day | 0.0 | HPAEC none | 0.0 |
| Two Way MLR 7 day | 0.0 | HPAEC TNF alpha + IL-1 | 0.0 |
| | | beta | |
| PBMC rest | 30.6 | Lung fibroblast none | 0.0 |
| PBMC PWM | 24.8 | Lung fibroblast TNF alpha + | 0.0 |
| | | IL-1beta | |
| PBMC PHA-L | 13.5 | Lung fibroblast IL-4 | 0.0 |
| Ramos (B cell) none | 0.0 | Lung fibroblast IL-9 | 0.0 |
| Ramos (B cell) | 0.0 | Lung fibroblast IL-13 | 0.0 |
| ionomycin | | | |
| B lymphocytes PWM | 100.0 | Lung fibroblast IFN | 0.0 |
| | | gamma | |
| B lymphocytes CD40L | 0.0 | Dermal fibroblast | 0.0 |
| and IL-4 | | CCD1070 rest | |
| EOL-1 dbcAMP | 0.0 | Dermal fibroblast | 0.0 |
| | | CCD1070 TNF alpha | |
| EOL-1 dbcAMP | 99.3 | Dermal fibroblast | 0.0 |
| PMA/ionomycin | | CCD1070 IL-1beta | |
| Dendritic cells none | 0.0 | Dermal fibroblast IFN | 0.0 |
| | | gamma | |
| Dendritic cells LPS | 22.4 | Dermal fibroblast IL-4 | 0.0 |
| Dendritic cells anti- | 36.3 | IBD Colitis 2 | 0.0 |
| CD40 | | | |
| Monocytes rest | 76.8 | IBD Crohn's | 22.2 |
| Monocytes LPS | 0.0 | Colon | 67.8 |
| Macrophages rest | 0.0 | Lung | 22.7 |
| Macrophages LPS | 0.0 | Thymus | 12.2 |
| HUVEC none | 0.0 | Kidney | 0.0 |
| HUVEC starved | 0.0 | | |

Panel 1.3D Summary: Ag3002 Significant expression of the NOV42a gene is limited to the mammary gland and the spleen (CTs=33-34). Thus, expression of this gene could be used to differentiate these samples from other samples on this panel and as a marker for these tissues.

Panel 4D Summary: Ag3002 Significant expression of the NOV42a gene is limited to activated B cells, an eosinophil cell line, and monocytes (CTs=33-35). Thus, this transcript could be used as a marker for phagocytic cell types.

NOV43

Expression of gene NOV43 was assessed using the primer-probe set Ag2987, described in Table ALA. Results of the RTQ-PCR runs are shown in Tables ALB, ALC, ALD and ALE.

TABLE ALA
Probe Name Ag2987

| | | | Start | |
|---|---|---|---|---|
| Primers | Sequences | Length | Position | SEQ ID NO: |
| Forward | 5′-ctacctcaccatctgctttctg-3′ | 22 | 860 | 1150 |
| Probe | TET-5′-tttctcaggactgccagctcttgatg-3′-TAMRA | 26 | 883 | 1151 |
| Reverse | 5′-tccatatcttgtaggccacact-3′ | 22 | 916 | 1152 |

TABLE ALB
CNS_neurodegeneration_v1.0

| | Rel. Exp. (%) Ag2987, | | Rel. Exp. (%) Ag2987, |
|---|---|---|---|
| Tissue Name | Run 211008970 | Tissue Name | Run 211008970 |
| AD 1 Hippo | 31.2 | Control (Path) 3 | 14.7 |
| | | Temporal Ctx | |
| AD 2 Hippo | 29.9 | Control (Path) 4 | 43.8 |
| | | Temporal Ctx | |
| AD 3 Hippo | 37.4 | AD 1 Occipital Ctx | 44.1 |
| AD 4 Hippo | 10.8 | AD 2 Occipital Ctx | 0.0 |
| | | (Missing) | |
| AD 5 hippo | 87.7 | AD 3 Occipital Ctx | 27.7 |
| AD 6 Hippo | 85.3 | AD 4 Occipital Ctx | 37.4 |
| Control 2 Hippo | 14.5 | AD 5 Occipital Ctx | 17.0 |
| Control 4 Hippo | 13.4 | AD 6 Occipital Ctx | 12.5 |
| Control (Path) 3 | 20.7 | Control 1 Occipital | 9.8 |
| Hippo | | Ctx | |
| AD 1 Temporal Ctx | 49.0 | Control 2 Occipital | 14.6 |
| | | Ctx | |
| AD 2 Temporal Ctx | 35.6 | Control 3 Occipital | 29.7 |
| | | Ctx | |
| AD 3 Temporal Ctx | 29.9 | Control 4 Occipital | 9.4 |
| | | Ctx | |
| AD 4 Temporal Ctx | 31.0 | Control (Path) 1 | 44.4 |
| | | Occipital Ctx | |
| AD 5 Inf Temporal | 100.0 | Control (Path) 2 | 13.2 |
| Ctx | | Occipital Ctx | |
| AD 5 SupTemporal | 64.2 | Control (Path) 3 | 5.8 |
| Ctx | | Occipital Ctx | |
| AD 6 Inf Temporal | 69.3 | Control (Path) 4 | 29.9 |
| Ctx | | Occipital Ctx | |
| AD 6 Sup Temporal | 96.6 | Control 1 Parietal | 19.3 |
| Ctx | | Ctx | |
| Control 1 Temporal | 17.0 | Control 2 Parietal | 67.8 |
| Ctx | | Ctx | |
| Control 2 Temporal | 10.6 | Control 3 Parietal | 23.7 |
| Ctx | | Ctx | |
| Control 3 Temporal | 26.4 | Control (Path) 1 | 23.0 |
| Ctx | | Parietal Ctx | |
| Control 4 Temporal | 11.0 | Control (Path) 2 | 36.1 |
| Ctx | | Parietal Ctx | |
| Control (Path) 1 | 35.6 | Control (Path) 3 | 16.2 |
| Temporal Ctx | | Parietal Ctx | |
| Control (Path) 2 | 35.4 | Control (Path) 4 | 45.4 |
| Temporal Ctx | | Parietal Ctx | |

TABLE ALC
Panel 1.3D

| | Rel. Exp. (%) Ag2987, | | Rel. Exp. (%) Ag2987, |
|---|---|---|---|
| Tissue Name | Run 166232814 | Tissue Name | Run 166232814 |
| Liver adenocarcinoma | 5.9 | Kidney (fetal) | 0.0 |
| Pancreas | 6.4 | Renal ca. 786-0 | 13.0 |
| Pancreatic ca. CAPAN 2 | 0.0 | Renal ca. A498 | 13.6 |
| Adrenal gland | 18.7 | Renal ca. RXF 393 | 28.1 |
| Thyroid | 5.6 | Renal ca. ACHN | 0.0 |
| Salivary gland | 40.9 | Renal ca. UO-31 | 0.0 |
| Pituitary gland | 23.5 | Renal ca. TK-10 | 5.4 |
| Brain (fetal) | 67.4 | Liver | 0.0 |
| Brain (whole) | 84.7 | Liver (fetal) | 4.8 |
| Brain (amygdala) | 39.5 | Liver ca. | 7.0 |
| | | (hepatoblast) HepG2 | |
| Brain (cerebellum) | 100.0 | Lung | 0.0 |
| Brain (hippocampus) | 21.8 | Lung (fetal) | 2.7 |
| Brain (substantia nigra) | 16.6 | Lung ca. (small cell) | 0.0 |
| | | LX-1 | |
| Brain (thalamus) | 96.6 | Lung ca. (small cell) | 0.0 |
| | | NCI-H69 | |
| Cerebral Cortex | 39.8 | Lung ca. (s.cell var.) | 10.3 |
| | | SHP-77 | |
| Spinal cord | 41.2 | Lung ca. (large | 13.0 |
| | | cell)NCI-H460 | |
| glio/astro U87-MG | 5.6 | Lung ca. (non-sm. | 0.0 |
| | | cell) A549 | |
| glio/astro U-118-MG | 51.8 | Lung ca. (non-s.cell) | 6.4 |
| | | NCI-H23 | |
| astrocytoma SW1783 | 0.0 | Lung ca. (non-s.cell) | 10.4 |
| | | HOP-62 | |
| neuro*; met SK-N-AS | 8.4 | Lung ca. (non-s.cl) | 0.0 |
| | | NCI-H522 | |
| astrocytoma SF-539 | 4.3 | Lung ca. (squam.) | 34.2 |
| | | SW 900 | |
| astrocytoma SNB-75 | 10.4 | Lung ca. (squam.) | 0.0 |
| | | NCI-H596 | |
| glioma SNB-19 | 28.5 | Mammary gland | 0.0 |
| glioma U251 | 10.2 | Breast ca.* (pl.ef) | 13.0 |
| | | MCF-7 | |
| glioma SF-295 | 76.8 | Breast ca.* (pl.ef) | 0.0 |
| | | MDA-MB-231 | |
| Heart (fetal) | 5.1 | Breast ca.* (pl.ef) | 0.0 |
| | | T47D | |
| Heart | 10.5 | Breast ca. BT-549 | 15.8 |
| Skeletal muscle (fetal) | 10.8 | Breast ca. MDA-N | 0.0 |
| Skeletal muscle | 15.7 | Ovary | 7.3 |
| Bone marrow | 5.0 | Ovarian ca. OVCAR-3 | 0.0 |
| Thymus | 0.0 | Ovarian ca. OVCAR-4 | 5.8 |
| Spleen | 11.3 | Ovarian ca. OVCAR-5 | 38.2 |
| Lymph node | 42.3 | Ovarian ca. OVCAR-8 | 31.4 |
| Colorectal | 8.4 | Ovarian ca. IGROV-1 | 10.2 |
| Stomach | 10.8 | Ovarian ca.* (ascites) | 54.0 |
| | | SK-OV-3 | |
| Small intestine | 34.9 | Uterus | 25.7 |
| Colon ca. SW480 | 0.0 | Placenta | 11.7 |
| Colon ca.* | 0.0 | Prostate | 18.9 |
| SW620(SW480 met) | | | |
| Colon ca. HT29 | 0.0 | Prostate ca.* (bone | 0.0 |
| | | met)PC-3 | |
| Colon ca. HCT-116 | 0.0 | Testis | 9.1 |
| Colon ca. CaCo-2 | 0.0 | Melanoma | 0.0 |
| | | Hs688(A).T | |
| Colon ca. | 4.8 | Melanoma* (met) | 0.0 |
| tissue(ODO3866) | | Hs688(B).T | |
| Colon ca. HCC-2998 | 0.0 | Melanoma UACC-62 | 11.9 |
| Gastric ca.* (liver met) | 31.2 | Melanoma M14 | 0.0 |
| NCI-N87 | | | |
| Bladder | 25.2 | Melanoma LOX | 5.2 |
| | | IMVI | |
| Trachea | 10.6 | Melanoma* (met) | 7.0 |
| | | SK-MEL-5 | |
| Kidney | 0.0 | Adipose | 27.4 |

TABLE ALD
Panel 4D

| | Rel. Exp. (%) | | Rel. Exp. (%) |
|---|---|---|---|
| | Ag2987, Run | | Ag2987, Run |
| Tissue Name | 164314632 | Tissue Name | 164314632 |
| Secondary Th1 act | 8.1 | HUVEC IL-1beta | 1.9 |
| Secondary Th2 act | 10.3 | HUVEC IFN gamma | 17.4 |
| Secondary Tr1 act | 2.2 | HUVEC TNF alpha + IFN | 9.6 |
| | | gamma | |
| Secondary Th1 rest | 8.8 | HUVEC TNF alpha + IL4 | 4.1 |
| Secondary Th2 rest | 10.0 | HUVEC IL-11 | 9.3 |
| Secondary Tr1 rest | 25.0 | Lung Microvascular EC | 31.9 |
| | | none | |
| Primary Th1 act | 4.0 | Lung Microvascular EC | 22.8 |
| | | TNF alpha + IL-1beta | |
| Primary Th2 act | 1.9 | Microvascular Dermal EC | 25.9 |
| | | none | |
| Primary Tr1 act | 4.5 | Microsvasular Dermal EC | 7.3 |
| | | TNF alpha + IL-1beta | |
| Primary Th1 rest | 40.1 | Bronchial epithelium | 33.4 |
| | | TNF alpha + IL1beta | |
| Primary Th2 rest | 39.5 | Small airway epithelium | 10.6 |
| | | none | |
| Primary Tr1 rest | 74.7 | Small airway epithelium | 77.4 |
| | | TNF alpha + IL-1beta | |
| CD45RA CD4 | 0.0 | Coronery artery SMC rest | 15.0 |
| lymphocyte act | | | |
| CD45RO CD4 | 15.4 | Coronery artery SMC | 3.0 |
| lymphocyte act | | TNF alpha + IL-1beta | |
| CD8 lymphocyte act | 6.1 | Astrocytes rest | 4.6 |
| Secondary CD8 | 16.3 | Astrocytes TNF alpha + | 0.0 |
| lymphocyte rest | | IL-1beta | |
| Secondary CD8 | 1.8 | KU-812 (Basophil) rest | 1.7 |
| lymphocyte act | | | |
| CD4 lymphocyte none | 10.0 | KU-812 (Basophil) | 20.9 |
| | | PMA/ionomycin | |
| 2ry Th1/Th2/Tr1_anti- | 13.7 | CCD1106 (Keratinocytes) | 8.4 |
| CD95 CH11 | | none | |
| LAK cells rest | 33.7 | CCD1106 (Keratinocytes) | 7.9 |
| | | TNF alpha + IL-1beta | |
| LAK cells IL-2 | 14.8 | Liver cirrhosis | 18.6 |
| LAK cells IL-2 + IL-12 | 14.9 | Lupus kidney | 15.3 |
| LAK cells IL-2 + IFN | 28.9 | NCI-H292 none | 15.0 |
| gamma | | | |
| LAK cells IL-2 + IL-18 | 14.4 | NCI-H292 IL-4 | 4.6 |
| LAK cells | 4.5 | NCI-H292 IL-9 | 13.2 |
| PMA/ionomycin | | | |
| NK Cells IL-2 rest | 11.3 | NCI-H292 IL-13 | 4.7 |
| Two Way MLR 3 day | 32.3 | NCI-H292 IFN gamma | 2.8 |
| Two Way MLR 5 day | 2.4 | HPAEC none | 8.0 |
| Two Way MLR 7 day | 0.0 | HPAEC TNF alpha + IL-1 | 10.2 |
| | | beta | |
| PBMC rest | 4.5 | Lung fibroblast none | 35.6 |
| PBMC PWM | 18.8 | Lung fibroblast TNF alpha + | 16.3 |
| | | IL-1beta | |
| PBMC PHA-L | 20.2 | Lung fibroblast IL-4 | 65.1 |
| Ramos (B cell) none | 1.4 | Lung fibroblast IL-9 | 55.5 |
| Ramos (B cell) | 5.6 | Lung fibroblast IL-13 | 57.4 |
| ionomycin | | | |
| B lymphocytes PWM | 19.2 | Lung fibroblast IFN | 100.0 |
| | | gamma | |
| B lymphocytes CD40L | 11.7 | Dermal fibroblast | 9.7 |
| and IL-4 | | CCD1070 rest | |
| EOL-1 dbcAMP | 59.9 | Dermal fibroblast | 29.7 |
| | | CCD1070 TNF alpha | |
| EOL-1 dbcAMP | 11.9 | Dermal fibroblast | 3.2 |
| PMA/ionomycin | | CCD1070 IL-1beta | |
| Dendritic cells none | 48.6 | Dermal fibroblast IFN | 11.4 |
| | | gamma | |
| Dendritic cells LPS | 17.7 | Dermal fibroblast IL-4 | 24.3 |
| Dendritic cells anti- | 29.5 | IBD Colitis 2 | 6.7 |
| CD40 | | | |
| Monocytes rest | 26.4 | IBD Crohn's | 4.5 |
| Monocytes LPS | 19.5 | Colon | 20.4 |
| Macrophages rest | 28.1 | Lung | 14.8 |
| Macrophages LPS | 8.9 | Thymus | 52.5 |
| HUVEC none | 9.9 | Kidney | 74.7 |
| HUVEC starved | 21.2 | | |

TABLE ALE
Panel CNS_1

| | Rel. Exp. (%) Ag2987, | | Rel. Exp. (%) Ag2987, |
|---|---|---|---|
| Tissue Name | Run 171670053 | Tissue Name | Run 171670053 |
| BA4 Control | 21.3 | BA17 PSP | 16.8 |
| BA4 Control2 | 15.2 | BA17 PSP2 | 4.4 |
| BA4 | 27.5 | Sub Nigra Control | 39.8 |
| Alzheimer's2 | | | |
| BA4 Parkinson's | 100.0 | Sub Nigra Control2 | 13.9 |
| BA4 | 66.9 | Sub Nigra | 34.6 |
| Parkinson's2 | | Alzheimer's2 | |
| BA4 | 33.2 | Sub Nigra | 51.4 |
| Huntington's | | Parkinson's2 | |
| BA4 | 31.4 | Sub Nigra | 69.3 |
| Huntington's2 | | Huntington's | |
| BA4 PSP | 3.9 | Sub Nigra | 22.7 |
| | | Huntington's2 | |
| BA4 PSP2 | 5.1 | Sub Nigra PSP2 | 17.2 |
| BA4 Depression | 31.6 | Sub Nigra | 23.0 |
| | | Depression | |
| BA4 | 23.3 | Sub Nigra | 17.1 |
| Depression2 | | Depression2 | |
| BA7 Control | 39.5 | Glob Palladus | 23.7 |
| | | Control | |
| BA7 Control2 | 6.0 | Glob Palladus | 6.3 |
| | | Control2 | |
| BA7 | 43.2 | Glob Palladus | 17.6 |
| Alzheimer's2 | | Alzheimer's | |
| BA7 Parkinson's | 33.2 | Glob Palladus | 20.2 |
| | | Alzheimer's2 | |
| BA7 | 61.1 | Glob Palladus | 76.8 |
| Parkinson's2 | | Parkinson's | |
| BA7 | 51.4 | Glob Palladus | 31.6 |
| Huntington's | | Parkinson's2 | |
| BA7 | 69.3 | Glob Palladus PSP | 4.4 |
| Huntington's2 | | | |
| BA7 PSP | 55.5 | Glob Palladus PSP2 | 7.8 |
| BA7 PSP2 | 39.8 | Glob Palladus | 19.6 |
| | | Depression | |
| BA7 Depression | 15.7 | Temp Pole Control | 25.7 |
| BA9 Control | 27.4 | Temp Pole Control2 | 15.5 |
| BA9 Control2 | 28.5 | Temp Pole | 11.7 |
| | | Alzheimer's | |
| BA9 Alzheimer's | 8.8 | Temp Pole | 16.4 |
| | | Alzheimer's2 | |
| BA9 | 42.3 | Temp Pole | 34.9 |
| Alzheimer's2 | | Parkinson's | |
| BA9 Parkinson's | 86.5 | Temp Pole | 38.7 |
| | | Parkinson's2 | |
| BA9 | 49.7 | Temp Pole | 65.5 |
| Parkinson's2 | | Huntington's | |
| BA9 | 45.1 | Temp Pole PSP | 3.1 |
| Huntington's | | | |
| BA9 | 40.1 | Temp Pole PSP2 | 12.2 |
| Huntington's2 | | | |
| BA9 PSP | 20.4 | Temp Pole | 19.1 |
| | | Depression2 | |
| BA9 PSP2 | 0.0 | Cing Gyr Control | 52.1 |
| BA9 Depression | 14.9 | Cing Gyr Control2 | 14.1 |
| BA9 | 17.7 | Cing Gyr | 13.1 |
| Depression2 | | Alzheimer's | |
| BA17 Control | 64.2 | Cing Gyr | 60.3 |
| | | Alzheimer's2 | |
| BA17 Control2 | 22.5 | Cing Gyr Parkinson's | 66.4 |
| BA17 | 52.1 | Cing Gyr | 25.5 |
| Alzheimer's2 | | Parkinson's2 | |
| BA17 | 85.3 | Cing Gyr | 85.3 |
| Parkinson's | | Huntington's | |
| BA17 | 94.0 | Cing Gyr | 43.2 |
| Parkinson's2 | | Huntington's2 | |
| BA17 | 70.7 | Cing Gyr PSP | 26.2 |
| Huntington's | | | |
| BA17 | 34.4 | Cing Gyr PSP2 | 6.2 |
| Huntington's2 | | | |
| BA17 | 46.7 | Cing Gyr Depression | 12.9 |
| Depression | | | |
| BA17 | 82.9 | Cing Gyr | 23.8 |
| Depression2 | | Depression2 | |

CNS_neurodegeneration_v1.0 Summary: Ag2987 The NOV43 gene exhibits significantly higher expression in the brains of Alzheimer's disease patients than normal controls. This is consistant with reports of increased purinoceptor expression in AD. Please see Panel 1.3D for discussion of utility of this gene in the central nervous system.

References:

Moore D, Iritani S, Chambers J, Emson P. Immunohistochemical localization of the P2Y1 purinergic receptor in Alzheimer's disease. Neuroreport Nov. 27, 2000;11(17):3799-803

The biological actions of extracellular nucleotides are mediated by two distinct classes of P2 receptor, P2X and P2Y. The G protein-coupled P2Y receptors comprise five mammalian subtypes, P2Y(1-11). The P2Y1 subtype is expressed abundantly throughout the human brain and is specifically localized to neuronal structures. In the present study, the distribution of the P2Y1 receptor was investigated in Alzheimer's disease (AD) brains. In contrast to control human brain, the P2Y1 receptor was localized to a number of characteristic AD structures such as neurofibrillary tangles, neuritic plaques and neuropil threads. Immunoblot analysis showed that this specific immunostaining observed over tangles was not a result of cross-reactivity between the anti-P2Y1 antiserum and abnormal tau protein, the major constituent of tangles. The significance of this altered P2Y1 cellular distribution in AD brains is at present unclear.

Panel 1.3D Summary: Ag2987 The NOV43 gene, a purinoceptor homolog, exhibits highly brain preferential expression in this panel. Purinoceptors found in GDNF sensitive sensory neurons mediate nociceptor function. Therefore, agents that block the action of this receptor may have utility in treating pain, either as analgesics or in inhibiting the establishment of chronic pain. In addition, adenosine plays a significant neuromodulatory role in brain regions such as the hippocampus, cortex, basal ganglia, and thalamus. Thus, this purinoceptor is localized in a position to participate with the action of adenosine in these brain regions. The NOV43 gene product may also influence Ca2+ mobilization, a function performed by other purinoceptors. Ca2+ mobilization is an important component of the molecular process leading to neurotransmitter release, such as dopamine and glutamate. P2Y receptors have been shown to affect the release of dopamine, a critical neurotransmitter deficient in Parkinson's disease. P2 receptor agonists are known to induce secretion. Therefore, agents that modulate NOV43 may be effective treatments for Parkinson's disease via effecting enhanced dopamine release. Furthermore, glutamate is the main excitatory amino acid neurotransmitter. Glutamate exerts excitotoxic neuronal damage and death in a number of pathological conditions, including stroke. Therefore, agents that inhibit this gene prodcut are likely to affect glutamate release in the brain and the subsequent cytotoxic action of glutamate in these regions. The overexpression of this gene in the brains of Alzheimer's disease patients in the CNS_neurodegeneration_v1.0 panel indicates that antagonists of this receptor may also have utility in countering the processes associated with this disease.

References:

Liu D M, Katnik C, Stafford M, Adams D J.P2Y purinoceptor activation mobilizes intracellular Ca2+ and induces a membrane current in rat intracardiac neurones. J Physiol Jul. 15, 2000;526 Pt 2:287-98

1. The mobilization of Ca2+ by purinoceptor activation and the relative contributions of intra- and extracellular sources of Ca2+ were investigated using microfluorimetric measurements of fura-2 loaded in cultured neurones from rat intracardiac ganglia. 2. Reverse transcriptase-polymerase chain reaction (RT-PCR) revealed expression of mRNA for the G protein-coupled P2Y2 and P2Y4 receptors. 3. Brief application of either 300 microM ATP or 300 microM UTP caused transient increases in [Ca2+]i of 277+/−22 nM and 267+/−39 nM, respectively. Removal of external Ca2+ did not significantly reduce these [Ca2+]i responses. 4. The order of purinoceptor agonist potency for [Ca2+]i increases was ATP=UTP>2-MeSATP>ADP>>adenosine, consistent with the profile for P2Y2 purinoceptors. ATP- and UTP-induced rises in [Ca2+]i were completely and reversibly blocked by 10 microM PPADS (a P2 purinoceptor antagonist) and partially inhibited by 100 microM suramin (a relatively non-specific purinoceptor antagonist). 5. In the presence of the endoplasmic reticulum Ca2+-ATPase inhibitor cyclopiazonic acid (10 microM) in Ca2+-free media, the [Ca2+]i responses evoked by ATP were progressively decreased and abolished. 6.ATP- and UTP-induced [Ca2+]i rises were insensitive to pertussis toxin, caffeine (5 mM) and ryanodine (10 microM) but were significantly reduced by U-73122, a phospholipase C (PLC) inhibitor. 7. In fura-2-loaded cells, perforated patch whole-cell recordings show that ATP and UTP evoked slow outward currents at −60 mV, concomitant with the rise in [Ca2+]i, in approximately 30% of rat intracardiac neurones. 8. In conclusion, these results suggest that in r intracardiac neurones, ATP binds to P2Y2 purinoceptors to transiently raise [Ca2+]i and activate an outward current. The signalling pathway appears to involve a PTX-insensitive G protein coupled to PLC generation of IP3 which triggers the release of Ca2+ from a ryanodine-insensitive Ca2+ store(s).

Driessen B, Bultmann R, Jurna I, Baldauf J.Depression of C fiber-evoked activity by intrathecally administered reactive red 2 in rat thalamic neurons. Brain Res Jun. 15, 1998;796(1-2):284-90

To investigate the possible role of spinal purinoceptors in nociception, the potent P2-purinoceptor antagonist reactive red 2 was studied in rats under urethane anesthesia in which nociceptive activity was elicited by electrical stimulation of afferent C fibers in the sural nerve and recorded from single neurons in the ventrobasal complex of the thalamus. Intrathecal (i.t.) application of reactive red 2 (6-200 micrograms) caused a dose-dependent reduction of the evoked activity in thalamic neurons. The estimated ED50 was 30 micrograms, and the maximum depression of nociceptive activity amounted to about 70% of the control activity at a dose of 100 micrograms. Morphine, administered i.t. at a maximally effective dose (80 micrograms), inhibited the evoked nociceptive activity by only up to 55% of the control activity. An i.t. co-injection of reactive red 2 (100 micrograms) and morphine (80 micrograms) caused a maximum reduction of the evoked thalamic activity by up to 85% of the control activity, thus, exceeding significantly the effect elicited by either drug alone. Similarly, i.t. co-injection of almost equipotent dosages of reactive red 2 (30 micrograms) and morphine (30 micrograms) caused a maximum reduction of the evoked activity by up to 72% of the control activity, which again exceeded significantly the effect of either drug alone. The results suggest that in rats reactive red 2 exerts antinociception by blockade of P2-purinoceptors in the spinal cord and, hence, support the idea that ATP may play an important role in spinal transmission of nociceptive signals. An activation of the spinal opioid system does not seem to contribute to the effect of reactive red 2 but might act additive or even synergistically with its antinociceptive action.

Krugel U, Kittner H, Franke H, Illes P. Stimulation of P2 receptors in the ventral tegmental area enhances dopaminergic mechanisms in vivo. Neuropharmacology June 2001;40(8): 1084-93

It has been shown that endogenous adenosine 5′-triphosphate (ATP) as well as its exogenously applied structural analogue, 2-methylthio ATP (2-MeSATP), facilitate the release of dopamine from axon terminals in the rat nucleus accumbens (NAc) by activating ATP-sensitive P2 receptors. In the present study, reversed microdialysis of 2-MeSATP (10 microM, 100 microM and 1 mM), or its microinjection (0.5, 5.0 and 50 pmol) into the ventral tegmental area (VTA), dose-dependently increased the local extracellular level of dopamine and the locomotion in the open field, respectively. These effects were abolished by the P2-receptor antagonist pyridoxalphosphate-6-azophenyl-2′,4′-disulfonic acid (PPADS). When applied alone, the antagonist decreased the basal dopamine concentration, indicating that endogenous ATP controls the somatodendritic release of dopamine. Repeated microinjections of 2-MeSATP (5 pmol) once daily for 4 days led to a reproducible locomotor stimulation in the open field. Conditioned locomotion was induced by re-exposure to the novel environment on the seventh day. A challenge with amphetamine (1 mg/kg intraperitoneally) on the eighth day enhanced the locomotor activity in the 2-MeSATP-treated group in the sense of a cross-sensitisation, but failed to do so in the control group. Neurons in the VTA were heavily stained with antibodies developed against the P2Y(1) subtype of P2 receptors. Taken together, our data suggest that P2 receptors (probably of the P2Y(1) subtype) are involved in the initiation of somatodendritic dopamine release in the VTA and thereby may have a profound influence on sensitisation and reward-motivated behaviour.

Fernandez-Alvarez J, Hillaire-Buys D, Loubatieres-Mariani M M, Gomis R, Petit P. P2 receptor agonists stimulate insulin release from human pancreatic islets. Pancreas January 2001;22(1):69-71

Although P2 receptors for adenosine 5′-triphosphate (ATP) and/or adenosine 5′-diphosphate (ADP) have been characterized in mammalian pancreatic beta cells, no evidence for an insulin-secreting effect of P2 receptor agonists has been reported as yet in humans. The present study aimed at investigating whether P2 receptor agonists could stimulate insulin release in human pancreatic islets obtained from brain-dead organ donors. Experiments were performed using different glucose concentrations and insulin was measured by radioimmunoassay. When the glucose concentration (8.3 mmol/L) was slightly stimulating for insulin release, alpha,beta-methylene ATP (200 micromol/L) and ADPbetaS (50 micromol/L) similarly amplified insulin secretion: both compounds induced a threefold increase in insulin response. In the presence of a nonstimulating glucose concentration (3.0 mmol/L), only alpha,beta-methylene ATP could induce a significant 1.4-fold increase in insulin release, ADPbetaS being completely ineffective. These results give evidence that P2 receptor agonists are effective in stimulating insulin release in humans, the effect of the P2Y agonist being essentially glucose dependent

Panel 4D Summary: Ag 2987 The NOV43 transcript is expressed in lung fibroblasts after treatment with IFNg, IL-4, IL-9 other cytokines (CTs=32). This gene is also expressed in small airway epithelium treated with the inflammatory cytokines TNF-a and IL-1. This expression profile suggests a role for this transcript in lung inflammation. Low but detectable expression of this transcript is found also in dermal fibroblasts, primary CD4 T cells, EOL and antigen presenting cells.

This transcript encodes for a PY2receptor like molecule. Expression of this receptor has been reported in several cell types including eosinophils (Ref.1) and lung epithelium where it has been shown to mediate Cl(−) secretion via an increase in intracellular calcium concentration (ref. 2). Thus, the NOV43 gene product may influence Ca2+ mobilization, a funtion performed by other purinoceptors, and therefore lead to activation or secretion processes. As suggested by ref.3, the release of nucleotides by damaged cells in inflammation can lead to the activation of purinoreceptors and of other cells present in the inflammed tissues, including lung epithelium in asthma, COPD, emphysema and the skin in psoriasis or other skin inflammatory diseases. This release can also result in the activation of antigen presenting cells and T cells, which contribute to the perpetuation of the inflammatory process. Therefore, modulation of the expression or activity of the protein encoded by the NOV43 gene may prevent or reduce the inflammation process in all of these diseases and other autoimmune diseases, including as inflammatory bowel diseases.

References:

Idzko M, Dichmann S, Panther E, Ferrari D, Herouy Y, Virchow C Jr, Luttmann W, Di Virgilio F, Norgauer J.

Functional characterization of P2Y and P2X receptors in human eosinophils. J Cell Physiol September 2001;188(3):329-36

Activation of purinoceptor by ATP induces in eosinophils various cell responses including calcium transients, actin polymerization, production of reactive oxygen metabolites, CD11b-expression, and chemotaxis. Here, the effect of ion channel-gated P2X and/or G protein-coupled P2Y receptor agonists ATP, ATPgammaS, alpha,beta-meATP, 2-MeSATP, BzATP, ADP, CTP, and UTP on the intracellular Ca(2+)-mobilization, actin polymerization, production of reactive oxygen metabolites, CD11b expression and chemotaxis of human eosinophils were measured and the biological activity was analyzed. Although all tested nucleotides were able to induce all these cell responses, the biological activity of the analyzed nucleotides were distinct. Agonists of the G protein-coupled P2Y receptors such as 2-MeSATP, UTP, and ADP have a higher biological activity for production of reactive oxygen metabolites, actin polymerization and chemotaxis in comparison to the ion channel-gated P2X agonists alphabeta-meATP, BzATP, and CTP. In contrast, P2Y and P2X agonist showed similar potencies in respect to intracellular calcium transient and CD11b up-regulation. This conclusion was further supported by experiments with receptor iso-type antagonist KN62, EGTA or with the G(i) protein-inactivating pertussis toxin. These findings indicate participation of different purinorecptors in the regulation of cell responses in eosinophils.

Laubinger W, Streubel G, Reiser G. Physiological evidence for a P2Y receptor responsive to diadenosine polyphosphates in human lung via Ca(2+) release studies in bronchial epithelial cells. Biochem Pharmacol Mar. 1, 2001;61(5):623-9

P2Y(2) receptors that are activated by the extracellular nucleotides ATP or UTP mediate Cl(−) secretion via an increase in [Ca(2+)](i) (intracellular calcium concentration). Therefore, in the lung of patients suffering from cystic fibrosis, inhalation of aerosolized UTP offers a way to circumvent the defect in Cl(−) secretion by the cystic fibrosis transmembrane conductance regulator. A possible alternative for the relatively unstable UTP in inhalation therapy is the more resistant diadenosine tetraphosphate (Ap(4)A). In human and rat lung membranes, Ap(4)A binds to P2 receptor sites coupled to G proteins. Here, we showed that Ap(4)A caused an increase in [Ca(2+)](i) with an EC(50) of 17 microM in human bronchial epithelial cells (HBE1). The [Ca(2+)](i) rise evoked by ATP and UTP was completely, but that induced by Ap(4)A only partially, caused by release of Ca(2+) from internal stores. Moreover, the potency of Ap(4)A to mobilize Ca(2+) was lower than that of ATP and UTP (EC(50) 1.5 and 1.8 microM, respectively), and the maximal increase in [Ca(2+)](i) was considerably smaller than that after ATP or UTP. In accordance with our previous results providing evidence for a common binding site for various diadenosine polyphosphates in lung membranes, all Ap(n)A analogues tested (n=3 to 6) caused a comparable [Ca(2+)](i) increase. Homologous or heterologous prestimulation largely diminished the increase in [Ca(2+)](i) found after a second pulse of either UTP or Ap(4)A. Although specific binding characteristics and functional responses of Ap(4)A on lung cells are in favor of a distinct receptor for Ap(4)A, the cross-talk between UTP and Ap(4)A in HBE1 cells and the only slight differences in Ca(2+) mobilization by ATP or UTP and Ap(4)A render it impossible at this point to state unequivocally whether there exists a distinct P2Y receptor specific for diadenosine polyphosphates in lung epithelia or whether Ap(4)A activates one of the nucleotide receptors already described.

Di Virgilio F, Chiozzi P, Ferrari D, Falzoni S, Sanz J M, Morelli A, Torboli M, Bolognesi G, Baricordi O R. Nucleotide receptors: an emerging family of regulatory molecules in blood cells. Blood Feb. 1, 2001;97(3):587-600

Nucleotides are emerging as an ubiquitous family of extracellular signaling molecules. It has been known for many years that adenosine diphosphate is a potent platelet aggregating factor, but it is now clear that virtually every circulating cell is responsive to nucleotides. Effects as different as proliferation or differentiation, chemotaxis, release of cytokines or lysosomal constituents, and generation of reactive oxygen or nitrogen species are elicited upon stimulation of blood cells with extracellular adenosine triphosphate (ATP). These effects are mediated through a specific class of plasma membrane receptors called purinergic P2 receptors that, according to the molecular structure, are further subdivided into 2 subfamilies: P2Y and P2X. ATP and possibly other nucleotides are released from damaged cells or secreted via nonlytic mechanisms. Thus, during inflammation or vascular damage, nucleotides may provide an important mechanism involved in the activation of leukocytes and platelets. However, the cell physiology of these receptors is still at its dawn, and the precise function of the multiple P2X and P2Y receptor subtypes remains to be understood.

Panel CNS_{—}1 Summary: Ag2987 The expression in this panel confirms expression of the NOV43 gene in the brain. Please see Panel 1.3D for discussion of utility of this gene in the central nervous system.

NOV44

Expression of gene NOV44 was assessed using the primer-probe sets Ag2988 and Ag2989, described in Tables AMA and AMB. Results of the RTQ-PCR runs are shown in Table AMC.

TABLE AMA
Probe Name Ag2988

| | | | Start | |
|---|---|---|---|---|
| Primers | Sequences | Length | Position | SEQ ID NO: |
| Forward | 5′-ctggccaacctatcctttattg-3′ | 22 | 195 | 1153 |
| Probe | TET-5′-tggctcctaaactcattgctgactca-3′-TAMRA | 26 | 238 | 1154 |
| Reverse | 5′-agatggttctcccctcatacaa-3′ | 22 | 264 | 1155 |

TABLE AMB
Probe Name Ag2989

| | | | Start | |
|---|---|---|---|---|
| Primers | Sequences | Length | Position | SEQ ID NO: |
| Forward | 5′-ctggccaacctatcctttattg-3′ | 22 | 195 | 1156 |
| Probe | TET-5′-tggctcctaaactcattgctgactca-3′-TAMRA | 26 | 238 | 1157 |
| Reverse | 5′-agatggttctcccctcatacaa-3′ | 22 | 264 | 1158 |

TABLE AMC
Panel 4D

| | Rel. Exp. (%) | | Rel. Exp. (%) |
|---|---|---|---|
| | Ag2988, Run | | Ag2988, Run |
| Tissue Name | 164523397 | Tissue Name | 164523397 |
| Secondary Th1 act | 0.0 | HUVEC IL-1beta | 0.0 |
| Secondary Th2 act | 0.0 | HUVEC IFN gamma | 0.0 |
| Secondary Tr1 act | 0.0 | HUVEC TNF alpha + IFN | 0.0 |
| | | gamma | |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC | 0.0 |
| | | none | |
| Primary Th1 act | 0.0 | Lung Microvascular EC | 0.0 |
| | | TNF alpha + IL-1beta | |
| Primary Th2 act | 0.0 | Microvascular Dermal EC | 0.0 |
| | | none | |
| Primary Tr1 act | 0.0 | Microsvasular Dermal EC | 0.0 |
| | | TNF alpha + IL-1beta | |
| Primary Th1 rest | 0.0 | Bronchial epithelium | 0.0 |
| | | TNF alpha + IL1beta | |
| Primary Th2 rest | 0.0 | Small airway epithelium | 0.0 |
| | | none | |
| Primary Tr1 rest | 0.0 | Small airway epithelium | 0.0 |
| | | TNF alpha + IL-1beta | |
| CD45RA CD4 | 0.0 | Coronery artery SMC rest | 0.0 |
| lymphocyte act | | | |
| CD45RO CD4 | 0.0 | Coronery artery SMC | 0.0 |
| lymphocyte act | | TNF alpha + IL-1beta | |
| CD8 lymphocyte act | 0.0 | Astrocytes rest | 0.0 |
| Secondary CD8 | 0.0 | Astrocytes TNF alpha + | 0.0 |
| lymphocyte rest | | IL-1beta | |
| Secondary CD8 | 0.0 | KU-812 (Basophil) rest | 0.0 |
| lymphocyte act | | | |
| CD4 lymphocyte none | 0.0 | KU-812 (Basophil) | 0.0 |
| | | PMA/ionomycin | |
| 2ry Th1/Th2/Tr1_anti- | 0.0 | CCD1106 (Keratinocytes) | 0.0 |
| CD95 CH11 | | none | |
| LAK cells rest | 0.0 | CCD1106 (Keratinocytes) | 0.0 |
| | | TNF alpha + IL-1beta | |
| LAK cells IL-2 | 0.0 | Liver cirrhosis | 100.0 |
| LAK cells IL-2 + IL-12 | 0.0 | Lupus kidney | 23.0 |
| LAK cells IL-2 + IFN | 0.0 | NCI-H292 none | 0.0 |
| gamma | | | |
| LAK cells IL-2 + IL-18 | 0.0 | NCI-H292 IL-4 | 0.0 |
| LAK cells | 0.0 | NCI-H292 IL-9 | 0.0 |
| PMA/ionomycin | | | |
| NK Cells IL-2 rest | 0.0 | NCI-H292 IL-13 | 0.0 |
| Two Way MLR 3 day | 0.0 | NCI-H292 IFN gamma | 0.0 |
| Two Way MLR 5 day | 0.0 | HPAEC none | 0.0 |
| Two Way MLR 7 day | 0.0 | HPAEC TNF alpha + IL-1 | 0.0 |
| | | beta | |
| PBMC rest | 0.0 | Lung fibroblast none | 0.0 |
| PBMC PWM | 0.0 | Lung fibroblast TNF alpha + | 0.0 |
| | | IL-1beta | |
| PBMC PHA-L | 0.0 | Lung fibroblast IL-4 | 0.0 |
| Ramos (B cell) none | 0.0 | Lung fibroblast IL-9 | 0.0 |
| Ramos (B cell) | 0.0 | Lung fibroblast IL-13 | 0.0 |
| ionomycin | | | |
| B lymphocytes PWM | 5.3 | Lung fibroblast IFN | 0.0 |
| | | gamma | |
| B lymphocytes CD40L | 0.0 | Dermal fibroblast | 0.0 |
| and IL-4 | | CCD1070 rest | |
| EOL-1 dbcAMP | 0.0 | Dermal fibroblast | 0.0 |
| | | CCD1070 TNF alpha | |
| EOL-1 dbcAMP | 0.0 | Dermal fibroblast | 0.0 |
| PMA/ionomycin | | CCD1070 IL-1beta | |
| Dendritic cells none | 0.0 | Dermal fibroblast IFN | 0.0 |
| | | gamma | |
| Dendritic cells LPS | 0.0 | Dermal fibroblast IL-4 | 0.0 |
| Dendritic cells anti- | 0.0 | IBD Colitis 2 | 19.9 |
| CD40 | | | |
| Monocytes rest | 0.0 | IBD Crohn's | 0.0 |
| Monocytes LPS | 0.0 | Colon | 0.0 |
| Macrophages rest | 0.0 | Lung | 0.0 |
| Macrophages LPS | 0.0 | Thymus | 10.2 |
| HUVEC none | 0.0 | Kidney | 0.0 |
| HUVEC starved | 24.5 | | |

CNS_neurodegeneration_v1.0 Summary: Ag2988/Ag2989 Expression of the NOV44 gene is low/undectectable in all samples on this panel (CTs>35). (Data not shown.)

Panel 1.3D Summary: Ag2988/Ag2989 Expression of the NOV44 gene is low/undetectable in all samples on this panel (CTs>35). (Data not shown.)

Panel 4D Summary: Ag2988 Significant expression of this gene is detected in a liver cirrhosis sample (CT=32.7). This gene encodes a putative GPCR; therefore, antibodies or small molecule therapeutics could reduce or inhibit fibrosis that occurs in liver cirrhosis. In addition, antibodies to this putative GPCR could also be used for the diagnosis of liver cirrhosis.

NOV45

Expression of gene NOV45 was assessed using the primer-probe sets Ag2979, Ag2982, Ag2981 and Ag2984, described in Tables ANA, ANB, ANC and AND. Results of the RTQ-PCR runs are shown in Table ANE.

TABLE ANA
Probe Name Ag2979

| | | | Start | |
|---|---|---|---|---|
| Primers | Sequences | Length | Position | SEQ ID NO: |
| Forward | 5′-tacttcttcgtgggctcctt-3′ | 20 | 827 | 1159 |
| Probe | TET-5′-aaggcagaacctgaagctggttctcc-3′-TAMRA | 26 | 862 | 1160 |
| Reverse | 5′-cattcacctcagtcgtgtcc-3′ | 20 | 901 | 1161 |

TABLE ANB
Probe Name Ag2982

| | | | Start | |
|---|---|---|---|---|
| Primers | Sequences | Length | Position | SEQ ID NO: |
| Forward | 5′-tacttcttcgtgggctcctt-3-40 | 20 | 827 | 1162 |
| Probe | TET-5′-aaggcagaacctgaagctggttctcc-3′-TAMRA | 26 | 862 | 1163 |
| Reverse | 5′-cattcacctcagtcgtgtcc-3′ | 20 | 901 | 1164 |

TABLE ANC
Probe Name Ag2981

| | | | Start | |
|---|---|---|---|---|
| Primers | Sequences | Length | Position | SEQ ID NO: |
| Forward | 5′-tacttcttcgtgggctcctt-3′ | 20 | 827 | 1165 |
| Probe | TET-5′-aaggcagaacctgaagctggttctcc-3′-TAMRA | 26 | 862 | 1166 |
| Reverse | 5′-cattcacctcagtcgtgtcc-3′ | 20 | 901 | 1167 |

TABLE AND
Probe Name Ag2984

| | | | Start | |
|---|---|---|---|---|
| Primers | Sequences | Length | Position | SEQ ID NO: |
| Forward | 5′-atcctccatcccatcttcaa-3′ | 20 | 284 | 1168 |
| Probe | TET-5′-cctcagccctgtgatgatgttttcct-3′-TAMRA | 26 | 307 | 1169 |
| Reverse | 5′-cgcttagaaagctcaggctt-3′ | 20 | 340 | 1170 |

TABLE ANE
Panel 4D

| | Rel. Exp. (%) | | Rel. Exp. (%) |
|---|---|---|---|
| | Ag2982, Run | | Ag2982, Run |
| Tissue Name | 158603041 | Tissue Name | 158603041 |
| Secondary Th1 act | 0.0 | HUVEC IL-1beta | 0.0 |
| Secondary Th2 act | 0.3 | HUVEC IFN gamma | 0.0 |
| Secondary Tr1 act | 0.0 | HUVEC TNF alpha + IFN | 0.0 |
| | | gamma | |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC | 0.0 |
| | | none | |
| Primary Th1 act | 0.0 | Lung Microvascular EC | 19.8 |
| | | TNF alpha + IL-1beta | |
| Primary Th2 act | 0.0 | Microvascular Dermal EC | 0.0 |
| | | none | |
| Primary Tr1 act | 0.0 | Microsvasular Dermal EC | 5.5 |
| | | TNF alpha + IL-1beta | |
| Primary Th1 rest | 0.0 | Bronchial epithelium | 58.2 |
| | | TNF alpha + IL1beta | |
| Primary Th2 rest | 0.0 | Small airway epithelium | 3.0 |
| | | none | |
| Primary Tr1 rest | 0.0 | Small airway epithelium | 100.0 |
| | | TNF alpha + IL-1beta | |
| CD45RA CD4 | 3.6 | Coronery artery SMC rest | 0.0 |
| lymphocyte act | | | |
| CD45RO CD4 | 0.0 | Coronery artery SMC | 0.0 |
| lymphocyte act | | TNF alpha + IL-1beta | |
| CD8 lymphocyte act | 0.0 | Astrocytes rest | 0.0 |
| Secondary CD8 | 0.0 | Astrocytes TNF alpha + | 0.8 |
| lymphocyte rest | | IL-1beta | |
| Secondary CD8 | 0.0 | KU-812 (Basophil) rest | 0.0 |
| lymphocyte act | | | |
| CD4 lymphocyte none | 0.0 | KU-812 (Basophil) | 0.0 |
| | | PMA/ionomycin | |
| 2ry Th1/Th2/Tr1_anti- | 2.4 | CCD1106 (Keratinocytes) | 0.3 |
| CD95 CH11 | | none | |
| LAK cells rest | 0.0 | CCD1106 (Keratinocytes) | 5.8 |
| | | TNF alpha + IL-1beta | |
| LAK cells IL-2 | 0.0 | Liver cirrhosis | 23.3 |
| LAK cells IL-2 + IL-12 | 0.0 | Lupus kidney | 2.7 |
| LAK cells IL-2 + IFN | 0.0 | NCI-H292 none | 0.0 |
| gamma | | | |
| LAK cells IL-2 + IL-18 | 0.0 | NCI-H292 IL-4 | 0.0 |
| LAK cells | 0.0 | NCI-H292 IL-9 | 0.0 |
| PMA/ionomycin | | | |
| NK Cells IL-2 rest | 0.0 | NCI-H292 IL-13 | 0.0 |
| Two Way MLR 3 day | 4.8 | NCI-H292 IFN gamma | 0.0 |
| Two Way MLR 5 day | 0.0 | HPAEC none | 0.0 |
| Two Way MLR 7 day | 5.7 | HPAEC TNF alpha + IL-1 | 0.7 |
| | | beta | |
| PBMC rest | 0.0 | Lung fibroblast none | 0.0 |
| PBMC PWM | 0.0 | Lung fibroblast TNF alpha + | 1.8 |
| | | IL-1beta | |
| PBMC PHA-L | 0.0 | Lung fibroblast IL-4 | 0.0 |
| Ramos (B cell) none | 0.0 | Lung fibroblast IL-9 | 0.0 |
| Ramos (B cell) | 0.0 | Lung fibroblast IL-13 | 0.0 |
| ionomycin | | | |
| B lymphocytes PWM | 3.1 | Lung fibroblast IFN | 0.0 |
| | | gamma | |
| B lymphocytes CD40L | 0.0 | Dermal fibroblast | 0.0 |
| and IL-4 | | CCD1070 rest | |
| EOL-1 dbcAMP | 0.7 | Dermal fibroblast | 4.1 |
| | | CCD1070 TNF alpha | |
| EOL-1 dbcAMP | 0.0 | Dermal fibroblast | 0.4 |
| PMA/ionomycin | | CCD1070 IL-1beta | |
| Dendritic cells none | 0.0 | Dermal fibroblast IFN | 0.0 |
| | | gamma | |
| Dendritic cells LPS | 0.0 | Dermal fibroblast IL-4 | 0.0 |
| Dendritic cells anti- | 0.0 | IBD Colitis 2 | 0.0 |
| CD40 | | | |
| Monocytes rest | 0.0 | IBD Crohn's | 0.0 |
| Monocytes LPS | 0.0 | Colon | 0.0 |
| Macrophages rest | 0.0 | Lung | 0.0 |
| Macrophages LPS | 0.0 | Thymus | 0.0 |
| HUVEC none | 0.0 | Kidney | 0.0 |
| HUVEC starved | 0.0 | | |

CNS_neurodegeneration_v1.0 Summary: Ag2979/Ag2982 Expression of the NOV45 gene is low/undetectable in all samples on this panel (CTs>35). (Data not shown.)

Panel 1.3D Summary: Ag2981/Ag2984 Expression of the NOV45 gene is low/undetectable in all samples on this panel (CTs>35). (Data not shown.)

Panel 4D Summary: Ag2982 Expression of the NOV45 gene is restricted to a few samples, with highest expression in small airway epithelium treated with TNF-alpha and IL-1 beta (CT=31.2). Significant expression is treated in bronchial epithelium and lung microvascular endothelial cells. Thus, expression of this gene could be used as a marker for activated epithelium. The expression in lung derived samples suggests that this protein may be involved in lung inflammatory disorders, including asthma and chronic obstructive pulmonary disorder. Results from a second experiment with the probe/primer set Ag2979 are not included because the amp plot indicates that there is a potential problem in one of the sample wells.

NOV46a and NOV46b

Expression of gene NOV46s and variant NOV46b was assessed using the primer-probe sets Ag2990 and Ag2991, described in Tables AOA and AOB. Results of the RTQ-PCR runs are shown in Tables AOC. Please note that variant NOV46B does not match the probe and primer set Ag2990.

TABLE AOA
Probe Name Ag2990

| | | | Start | |
|---|---|---|---|---|
| Primers | Sequences | Length | Position | SEQ ID NO: |
| Forward | 5′-cggaactgaggagactctttg-3′ | 21 | 59 | 1171 |
| Probe | TET-5′-tacaagcagaccttgagcctcacggt-3′-TAMRA | 26 | 81 | 1172 |
| Reverse | 5′-gagcacaactgcgtttcct-3′ | 19 | 140 | 1173 |

TABLE AOB
Probe Name Ag2991

| | | | Start | |
|---|---|---|---|---|
| Primers | Sequences | Length | Position | SEQ ID NO: |
| Forward | 5′-tggacagggaagtcttattttg-3′ | 22 | 742 | 1174 |
| Probe | TET-5′-tttcctgtccgctcttaacagcagtg-3′-TAMRA | 26 | 785 | 1175 |
| Reverse | 5′-agcccacgaagaagtaaatgat-3′ | 22 | 819 | 1176 |

TABLE AOC
Panel 4D

| | Rel. | Rel. | | Rel. | Rel. |
|---|---|---|---|---|---|
| | Exp. (%) | Exp. (%) | | Exp. (%) | Exp. (%) |
| | Ag2990, | Ag2991, | | Ag2990, | Ag2991, |
| | Run | Run | | Run | Run |
| Tissue Name | 164524407 | 164315033 | Tissue Name | 164524407 | 164315033 |
| Secondary Th1 act | 0.0 | 0.0 | HUVEC IL-1beta | 0.0 | 0.0 |
| Secondary Th2 act | 0.0 | 0.0 | HUVEC IFN | 0.0 | 0.0 |
| | | | gamma | | |
| Secondary Tr1 act | 0.0 | 0.0 | HUVEC TNF | 0.0 | 0.0 |
| | | | alpha + IFN | | |
| | | | gamma | | |
| Secondary Th1 rest | 0.0 | 0.0 | HUVEC TNF | 0.0 | 0.0 |
| | | | alpha + IL4 | | |
| Secondary Th2 rest | 0.0 | 0.0 | HUVEC IL-11 | 0.0 | 0.0 |
| Secondary Tr1 rest | 0.0 | 0.0 | Lung | 0.0 | 0.0 |
| | | | Microvascular EC | | |
| | | | none | | |
| Primary Th1 act | 0.0 | 0.0 | Lung | 3.1 | 23.2 |
| | | | Microvascular EC | | |
| | | | TNF alpha + IL- | | |
| | | | 1beta | | |
| Primary Th2 act | 0.0 | 0.0 | Microvascular | 0.0 | 0.0 |
| | | | Dermal EC none | | |
| Primary Tr1 act | 0.0 | 0.0 | Microsvasular | 0.0 | 11.6 |
| | | | Dermal EC | | |
| | | | TNF alpha + IL- | | |
| | | | 1beta | | |
| Primary Th1 rest | 0.0 | 0.0 | Bronchial | 12.3 | 100.0 |
| | | | epithelium | | |
| | | | TNF alpha + | | |
| | | | IL1beta | | |
| Primary Th2 rest | 0.0 | 0.0 | Small airway | 0.0 | 12.1 |
| | | | epithelium none | | |
| Primary Tr1 rest | 0.0 | 0.0 | Small airway | 11.7 | 98.6 |
| | | | epithelium | | |
| | | | TNF alpha + IL- | | |
| | | | 1beta | | |
| CD45RA CD4 | 0.0 | 0.0 | Coronery artery | 0.0 | 0.0 |
| lymphocyte act | | | SMC rest | | |
| CD45RO CD4 | 0.0 | 0.0 | Coronery artery | 0.0 | 0.0 |
| lymphocyte act | | | SMC TNF alpha + | | |
| | | | IL-1beta | | |
| CD8 lymphocyte | 0.0 | 0.0 | Astrocytes rest | 0.0 | 0.0 |
| act | | | | | |
| Secondary CD8 | 0.0 | 0.0 | Astrocytes | 0.0 | 0.0 |
| lymphocyte rest | | | TNF alpha + IL- | | |
| | | | 1beta | | |
| Secondary CD8 | 0.0 | 0.0 | KU-812 | 0.0 | 0.0 |
| lymphocyte act | | | (Basophil) rest | | |
| CD4 lymphocyte | 0.0 | 0.0 | KU-812 | 23.8 | 5.0 |
| none | | | (Basophil) | | |
| | | | PMA/ionomycin | | |
| 2ry | 0.0 | 0.0 | CCD1106 | 0.0 | 0.0 |
| Th1/Th2/Tr1_anti- | | | (Keratinocytes) | | |
| CD95 CH11 | | | none | | |
| LAK cells rest | 0.0 | 0.0 | CCD1106 | 0.0 | 10.7 |
| | | | (Keratinocytes) | | |
| | | | TNF alpha + IL- | | |
| | | | 1beta | | |
| LAK cells IL-2 | 0.0 | 0.0 | Liver cirrhosis | 100.0 | 18.0 |
| LAK cells IL-2 + IL- | 0.0 | 0.0 | Lupus kidney | 0.0 | 0.0 |
| 12 | | | | | |
| LAK cells IL- | 0.0 | 0.0 | NCI-H292 none | 0.0 | 0.0 |
| 2 + IFN gamma | | | | | |
| LAK cells IL-2 + | 0.0 | 0.0 | NCI-H292 IL-4 | 0.0 | 3.8 |
| IL-18 | | | | | |
| LAK cells | 0.0 | 0.0 | NCI-H292 IL-9 | 0.0 | 0.0 |
| PMA/ionomycin | | | | | |
| NK Cells IL-2 rest | 27.5 | 0.0 | NCI-H292 IL-13 | 0.0 | 0.0 |
| Two Way MLR 3 | 0.0 | 0.0 | NCI-H292 IFN | 0.0 | 0.0 |
| day | | | gamma | | |
| Two Way MLR 5 | 2.1 | 0.0 | HPAEC none | 0.0 | 0.0 |
| day | | | | | |
| Two Way MLR 7 | 0.0 | 0.0 | HPAEC TNF | 0.0 | 0.0 |
| day | | | alpha + IL-1beta | | |
| PBMC rest | 0.0 | 0.0 | Lung fibroblast | 0.0 | 0.0 |
| | | | none | | |
| PBMC PWM | 0.0 | 0.0 | Lung fibroblast | 0.0 | 2.0 |
| | | | TNF alpha + IL-1 | | |
| | | | beta | | |
| PBMC PHA-L | 0.0 | 0.0 | Lung fibroblast | 0.0 | 0.0 |
| | | | IL-4 | | |
| Ramos (B cell) | 0.0 | 0.0 | Lung fibroblast | 0.0 | 0.0 |
| none | | | IL-9 | | |
| Ramos (B cell) | 0.0 | 0.0 | Lung fibroblast | 0.0 | 0.0 |
| ionomycin | | | IL-13 | | |
| B lymphocytes | 0.0 | 0.0 | Lung fibroblast | 0.0 | 0.0 |
| PWM | | | IFN gamma | | |
| B lymphocytes | 0.0 | 0.0 | Dermal fibroblast | 0.0 | 0.0 |
| CD40L and IL-4 | | | CCD1070 rest | | |
| EOL-1 dbcAMP | 0.0 | 0.0 | Dermal fibroblast | 0.0 | 4.0 |
| | | | CCD1070 TNF | | |
| | | | alpha | | |
| EOL-1 dbcAMP | 0.0 | 0.0 | Dermal fibroblast | 0.0 | 0.0 |
| PMA/ionomycin | | | CCD1070 IL-1 | | |
| | | | beta | | |
| Dendritic cells | 0.0 | 0.0 | Dermal fibroblast | 0.0 | 0.0 |
| none | | | IFN gamma | | |
| Dendritic cells LPS | 0.0 | 0.0 | Dermal fibroblast | 7.0 | 0.0 |
| | | | IL-4 | | |
| Dendritic cells anti- | 0.0 | 0.0 | IBD Colitis 2 | 0.0 | 6.2 |
| CD40 | | | | | |
| Monocytes rest | 0.0 | 0.0 | IBD Crohn's | 0.0 | 0.0 |
| Monocytes LPS | 0.0 | 0.0 | Colon | 0.0 | 0.0 |
| Macrophages rest | 0.0 | 0.0 | Lung | 0.0 | 0.0 |
| Macrophages LPS | 0.0 | 4.8 | Thymus | 0.0 | 0.0 |
| HUVEC none | 0.0 | 0.0 | Kidney | 0.0 | 0.0 |
| HUVEC starved | 0.0 | 0.0 | | | |

CNS_neurodegeneration_v1.0 Summary: Ag2990/Ag2991 Expression of the NOV46a gene is low/undetectable in all samples on this panel (CTs>35). (Data not shown.)

Panel 1.3D Summary: Ag2990/Ag2991 Expression of the NOV46a gene is low/undetectable in all samples on this panel (CTs>35). (Data not shown.)

Panel 4D Summary: Ag2990/Ag2991 Expression of the NOV46a gene is restricted to liver cirrhosis and TNFalpha+IL1 beta treated bronchial and small airway epithelium. This expression profile suggests that antibodies or small molecule therapeutics designed with the putative protein encoded by this gene could reduce or inhibit fibrosis that occurs in liver cirrhosis. In addition, antibodies to this putative protein product could also be used for the diagnosis of liver cirrhosis. In addition, the expression of this gene in tissues derived from the lung suggests that this gene product may be involved in pathological and inflammatory lung disorders that include chronic obstructive pulmonary disease, asthma, allergy and emphysema. A second experiment with Ag290 shows low/undetectable in all samples on this panel (CTs>35). (Data not shown.)

NOV46d

Expression of gene NOV46d was assessed using the primer-probe sets Ag2992 and Ag513, described in Tables APA and APB. Results of the RTQ-PCR runs are shown in Tables APC and APD.

TABLE APA
Probe Name Ag2992

| | | | Start | |
|---|---|---|---|---|
| Primers | Sequences | Length | Position | SEQ ID NO: |
| Forward | 5′-agggctggtcttcctcttct-3′ | 20 | 703 | 1177 |
| Probe | TET-5′-cccctcagcattcagggattcctatt-3′-TAMRA | 26 | 731 | 1178 |
| Reverse | 5′-agtcatccaaatccttctcgat-3′ | 22 | 764 | 1179 |

TABLE APB
Probe Name Ag513

| | | | Start | |
|---|---|---|---|---|
| Primers | Sequences | Length | Position | SEQ ID NO: |
| Forward | 5′-tatctgtggttctctgtgggttca-3′ | 24 | 600 | 1180 |
| Probe | TET-5′-atccacatatgatcctgacaagcaggaccag-3′-TAMRA | 31 | 626 | 1180 |
| Reverse | 5′-tggtcagcggcatcttctg-3′ | 19 | 659 | 1182 |

TABLE APC
Panel 1.1

| | Rel. Exp. (%) Ag513, | | Rel. Exp. (%) Ag513, |
|---|---|---|---|
| Tissue Name | Run 124882567 | Tissue Name | Run 124882567 |
| Adrenal gland | 0.0 | Renal ca. UO-31 | 0.0 |
| Bladder | 0.0 | Renal ca. RXF 393 | 0.0 |
| Brain (amygdala) | 0.0 | Liver | 0.0 |
| Brain (cerebellum) | 0.0 | Liver (fetal) | 0.0 |
| Brain (hippocampus) | 0.0 | Liver ca. | 0.0 |
| | | (hepatoblast) HepG2 | |
| Brain (substantia | 0.0 | Lung | 0.0 |
| nigra) | | | |
| Brain (thalamus) | 0.0 | Lung (fetal) | 0.0 |
| Cerebral Cortex | 0.0 | Lung ca. (non-s.cell) | 0.0 |
| | | HOP-62 | |
| Brain (fetal) | 0.0 | Lung ca. (large | 0.0 |
| | | cell)NCI-H460 | |
| Brain (whole) | 0.0 | Lung ca. (non-s.cell) | 0.0 |
| | | NCI-H23 | |
| glio/astro U-118-MG | 0.0 | Lung ca. (non-s.cl) | 0.0 |
| | | NCI-H522 | |
| astrocytoma SF-539 | 0.0 | Lung ca. (non-sm. | 0.0 |
| | | cell) A549 | |
| astrocytoma SNB-75 | 0.0 | Lung ca. (s.cell var.) | 0.0 |
| | | SHP-77 | |
| astrocytoma SW1783 | 0.0 | Lung ca. (small cell) | 0.0 |
| | | LX-1 | |
| glioma U251 | 0.0 | Lung ca. (small cell) | 81.8 |
| | | NCI-H69 | |
| glioma SF-295 | 0.0 | Lung ca. (squam.) | 0.0 |
| | | SW 900 | |
| glioma SNB-19 | 0.0 | Lung ca. (squam.) | 1.4 |
| | | NCI-H596 | |
| glio/astro U87-MG | 0.0 | Lymph node | 0.0 |
| neuro*; met SK-N-AS | 0.0 | Spleen | 0.0 |
| Mammary gland | 0.0 | Thymus | 0.0 |
| Breast ca. BT-549 | 0.0 | Ovary | 0.0 |
| Breast ca. MDA-N | 0.0 | Ovarian ca. IGROV-1 | 0.0 |
| Breast ca.* (pl.ef) | 0.0 | Ovarian ca. OVCAR-3 | 0.0 |
| T47D | | | |
| Breast ca.* (pl.ef) | 0.0 | Ovarian ca. OVCAR-4 | 0.0 |
| MCF-7 | | | |
| Breast ca.* (pl.ef) | 0.0 | Ovarian ca. OVCAR-5 | 100.0 |
| MDA-MB-231 | | | |
| Small intestine | 0.0 | Ovarian ca. OVCAR-8 | 0.0 |
| Colorectal | 0.0 | Ovarian ca.* (ascites) | 0.0 |
| | | SK-OV-3 | |
| Colon ca. HT29 | 0.0 | Pancreas | 0.0 |
| Colon ca. CaCo-2 | 0.0 | Pancreatic ca. | 0.0 |
| | | CAPAN 2 | |
| Colon ca. HCT-15 | 1.5 | Pituitary gland | 0.0 |
| Colon ca. HCT-116 | 0.0 | Placenta | 0.0 |
| Colon ca. HCC-2998 | 0.0 | Prostate | 0.0 |
| Colon ca. SW480 | 0.0 | Prostate ca.* (bone | 0.0 |
| | | met) PC-3 | |
| Colon ca.* SW620 | 0.0 | Salivary gland | 0.0 |
| (SW480 met) | | | |
| Stomach | 0.0 | Trachea | 0.0 |
| Gastric ca. (liver met) | 0.0 | Spinal cord | 0.0 |
| NCI-N87 | | | |
| Heart | 0.0 | Testis | 0.0 |
| Skeletal muscle (Fetal) | 0.0 | Thyroid | 0.0 |
| Skeletal muscle | 0.0 | Uterus | 0.0 |
| Endothelial cells | 0.0 | Melanoma M14 | 0.0 |
| Heart (Fetal) | 0.0 | Melanoma LOX | 0.0 |
| | | IMVI | |
| Kidney | 0.0 | Melanoma UACC-62 | 0.0 |
| Kidney (fetal) | 0.0 | Melanoma SK-MEL- | 0.0 |
| | | 28 | |
| Renal ca. 786-0 | 0.0 | Melanoma* (met) | 0.0 |
| | | SK-MEL-5 | |
| Renal ca. A498 | 0.0 | Melanoma | 0.0 |
| | | Hs688(A).T | |
| Renal ca. ACHN | 0.0 | Melanoma* (met) | 0.0 |
| | | Hs688(B).T | |
| Renal ca. TK-10 | 0.0 | | |

TABLE APD
Panel 1.2

| | Rel. Exp. (%) Ag513, | | Rel. Exp. (%) Ag513, |
|---|---|---|---|
| Tissue Name | Run 129119406 | Tissue Name | Run 129119406 |
| Endothelial cells | 0.0 | Renal ca. 786-0 | 0.0 |
| Heart (Fetal) | 0.0 | Renal ca. A498 | 0.0 |
| Pancreas | 0.0 | Renal ca. RXF 393 | 0.0 |
| Pancreatic ca. CAPAN 2 | 0.0 | Renal ca. ACHN | 0.0 |
| Adrenal Gland | 0.0 | Renal ca. UO-31 | 0.0 |
| Thyroid | 0.0 | Renal ca. TK-10 | 0.0 |
| Salivary gland | 0.0 | Liver | 0.0 |
| Pituitary gland | 0.0 | Liver (fetal) | 0.0 |
| Brain (fetal) | 0.0 | Liver ca. | 0.0 |
| | | (hepatoblast) HepG2 | |
| Brain (whole) | 0.0 | Lung | 0.0 |
| Brain (amygdala) | 0.0 | Lung (fetal) | 0.0 |
| Brain (cerebellum) | 0.0 | Lung ca. (small cell) | 0.0 |
| | | LX-1 | |
| Brain (hippocampus) | 0.0 | Lung ca. (small cell) | 100.0 |
| | | NCI-H69 | |
| Brain (thalamus) | 0.0 | Lung ca. (s.cell var.) | 0.0 |
| | | SHP-77 | |
| Cerebral Cortex | 0.0 | Lung ca. (large | 0.0 |
| | | cell)NCI-H460 | |
| Spinal cord | 0.0 | Lung ca. (non-sm. | 0.0 |
| | | cell) A549 | |
| glio/astro U87-MG | 0.0 | Lung ca. (non-s.cell) | 0.0 |
| | | NCI-H23 | |
| glio/astro U-118-MG | 0.0 | Lung ca. (non-s.cell) | 0.0 |
| | | HOP-62 | |
| astrocytoma SW1783 | 0.0 | Lung ca. (non-s.cl) | 0.0 |
| | | NCI-H522 | |
| neuro*; met SK-N-AS | 0.0 | Lung ca. (squam.) | 0.0 |
| | | SW 900 | |
| astrocytoma SF-539 | 0.0 | Lung ca. (squam.) | 40.6 |
| | | NCI-H596 | |
| astrocytoma SNB-75 | 0.0 | Mammary gland | 0.0 |
| glioma SNB-19 | 0.0 | Breast ca.* (pl.ef) | 0.0 |
| | | MCF-7 | |
| glioma U251 | 0.0 | Breast ca.* (pl.ef) | 0.0 |
| | | MDA-MB-231 | |
| glioma SF-295 | 0.0 | Breast ca.* (pl. ef) | 0.0 |
| | | T47D | |
| Heart | 0.0 | Breast ca. BT-549 | 0.0 |
| Skeletal Muscle | 0.0 | Breast ca. MDA-N | 0.0 |
| Bone marrow | 0.0 | Ovary | 0.0 |
| Thymus | 0.0 | Ovarian ca. OVCAR-3 | 0.0 |
| Spleen | 0.0 | Ovarian ca. OVCAR-4 | 0.0 |
| Lymph node | 0.0 | Ovarian ca. OVCAR-5 | 91.4 |
| Colorectal Tissue | 0.0 | Ovarian ca. OVCAR-8 | 0.0 |
| Stomach | 0.0 | Ovarian ca. IGROV-1 | 0.0 |
| Small intestine | 0.0 | Ovarian ca (ascites) | 0.0 |
| | | SK-OV-3 | |
| Colon ca. SW480 | 0.0 | Uterus | 0.0 |
| Colon ca.* SW620 | 0.0 | Placenta | 0.0 |
| (SW480 met) | | | |
| Colon ca. HT29 | 0.0 | Prostate | 0.0 |
| Colon ca. HCT-116 | 0.0 | Prostate ca.* (bone | 0.0 |
| | | met) PC-3 | |
| Colon ca. CaCo-2 | 0.0 | Testis | 0.0 |
| Colon ca. Tissue | 59.0 | Melanoma | 0.0 |
| (ODO3866) | | Hs688(A).T | |
| Colon ca. HCC-2998 | 0.0 | Melanoma* (met) | 2.0 |
| | | Hs688(B).T | |
| Gastric ca.* (liver met) | 0.0 | Melanoma UACC-62 | 0.0 |
| NCI-N87 | | | |
| Bladder | 0.0 | Melanoma M14 | 0.0 |
| Trachea | 0.0 | Melanoma LOX | 0.0 |
| | | IMVI | |
| Kidney | 0.0 | Melanoma* (met) | 0.0 |
| | | SK-MEL-5 | |
| Kidney (fetal) | 0.0 | | |

CNS_neurodegeneration_v1.0 Summary: Ag2992 Expression of the NOV46d gene is low/undetectable in all samples on this panel (CTs>35). (Data not shown.)

Panel 1.1 Summary: Ag513 Expression of the NOV46d gene is limited to two samples derived from lung cancer and ovarian cancer cell lines (CTs=31-32). Thus, expression of this gene could be used to differentiate between these sample and other samples on this panel and as a marker to detect the presence of lung and ovarian cancers. Furthermore, therapeutic modulation of the expression or function of this gene may be effective in the treatment of lung and ovarian cancers.

Panel 1.2 Summary: Ag513 Expression of the NOV46d gene is restricted to samples derived from lung cancer, ovarian cancer, and colon cancer cell lines (CTs=31-32). This expression profile is in agreement with the expression seen in Panel 1.1. Thus, expression of this gene could be used to differentiate between these sample and other samples on this panel and as a marker to detect the presence of these cancers. Furthermore, therapeutic modulation of the expression or function of this gene may be effective in the treatment of lung, ovarian, and colon cancers.

Panel 1.3D Summary: Ag2992 Expression of the NOV46d gene is low/undetectable in all samples on this panel (CTs>35). (Data not shown.)

Panel 4D Summary: Ag2992 Expression of the NOV46d gene is low/undetectable in all samples on this panel (CTs>35). (Data not shown.)

NOV46c

Expression of gene NOV46c was assessed using the primer-probe set Ag2985, described in Table AQA.

TABLE AQA
Probe Name Ag2985

| | | | Start | |
|---|---|---|---|---|
| Primers | Sequences | Length | Position | SEQ ID NO: |
| Forward | 5′-tctgtcatccccatctccaaa-3′ | 20 | 285 | 183 |
| Probe | TET-5′-atcctcattcctgtgatgacctttct-3′-TAMRA | 26 | 305 | 1184 |
| Reverse | 5′-tcatggcactcagaaagctc-3′ | 20 | 346 | 1185 |

CNS_neurodegeneration_v1.0 Summary: Ag2985 Expression of the NOV46c gene is low/undetectable in all samples on this panel (CTs>35). (Data not shown.)

Panel 4D Summary: Ag2985 Expression of the NOV46c gene is low/undetectable in all samples on this panel (CTs>35). (Data not shown.)

NOV47

Expression of gene NOV47 was assessed using the primer-probe set Ag2993, described in Table ARA. Results of the RTQ-PCR runs are shown in Tables ARB, ARC and ARD.

TABLE ARA
Probe Name Ag2993

| | | | Start | |
|---|---|---|---|---|
| Primers | Sequences | Length | Position | SEQ ID NO: |
| Forward | 5-40 -gagggttactgctttcacagaa-3′ | 22 | 161 | 1186 |
| Probe | TET-5′-tgacttcacatgccataatggcactg-3′-TAMRA | 26 | 211 | 1187 |
| Reverse | 5′-ctccctgtagatggacttgct-3′ | 21 | 239 | 1188 |

TABLE ARB
CNS_neurodegeneration_v1.0

| | Rel. Exp. (%) Ag2993, | | Rel. Exp. (%) Ag2993, |
|---|---|---|---|
| Tissue Name | Run 211009463 | Tissue Name | Run 211009463 |
| AD 1 Hippo | 11.9 | Control (Path) 3 | 10.8 |
| | | Temporal Ctx | |
| AD 2 Hippo | 43.2 | Control (Path) 4 | 55.5 |
| | | Temporal Ctx | |
| AD 3 Hippo | 5.3 | AD 1 Occipital Ctx | 38.4 |
| AD 4 Hippo | 15.2 | AD 2 Occipital Ctx | 0.0 |
| | | (Missing) | |
| AD 5 Hippo | 100.0 | AD 3 Occipital Ctx | 7.1 |
| AD 6 Hippo | 33.7 | AD 4 Occipital Ctx | 33.2 |
| Control 2 Hippo | 25.0 | AD 5 Occipital Ctx | 21.6 |
| Control 4 Hippo | 18.4 | AD 6 Occipital Ctx | 16.0 |
| Control (Path) 3 | 5.3 | Control 1 Occipital | 2.2 |
| Hippo | | Ctx | |
| AD 1 Temporal Ctx | 2.9 | Control 2 Occipital | 39.2 |
| | | Ctx | |
| AD 2 Temporal Ctx | 42.3 | Control 3 Occipital | 16.3 |
| | | Ctx | |
| AD 3 Temporal Ctx | 11.9 | Control 4 Occipital | 6.4 |
| | | Ctx | |
| AD 4 Temporal Ctx | 45.4 | Control (Path) 1 | 64.6 |
| | | Occipital Ctx | |
| AD 5 Inf Temporal | 86.5 | Control (Path) 2 | 15.8 |
| Ctx | | Occipital Ctx | |
| AD 5 Sup Temporal | 39.5 | Control (Path) 3 | 3.1 |
| Ctx | | Occipital Ctx | |
| AD 6 Inf Temporal | 24.8 | Control (Path) 4 | 27.9 |
| Ctx | | Occipital Ctx | |
| AD 6 Sup Temporal | 30.1 | Control 1 Parietal | 15.1 |
| Ctx | | Ctx | |
| Control 1 Temporal | 8.7 | Control 2 Parietal | 31.9 |
| Ctx | | Ctx | |
| Control 2 Temporal | 26.6 | Control 3 Parietal | 26.6 |
| Ctx | | Ctx | |
| Control 3 Temporal | 13.5 | Control (Path) 1 | 13.3 |
| Ctx | | Parietal Ctx | |
| Control 3 Temporal | 6.2 | Control (Path) 2 | 45.7 |
| Ctx | | Parietal Ctx | |
| Control (Path) 1 | 38.4 | Control (Path) 3 | 6.7 |
| Temporal Ctx | | Parietal Ctx | |
| Control (Path) 2 | 34.9 | Control (Path) 4 | 36.3 |
| Temporal Ctx | | Parietal Ctx | |

TABLE ARC
Panel 1.3D

| | Rel. Exp. (%) Ag2993, | | Rel. Exp. (%) Ag2993, |
|---|---|---|---|
| Tissue Name | Run 166230386 | Tissue Name | Run 166230386 |
| Liver adenocarcinoma | 23.3 | Kidney (fetal) | 19.5 |
| Pancreas | 0.0 | Renal ca. 786-0 | 20.7 |
| Pancreatic ca. CAPAN 2 | 34.2 | Renal ca. A498 | 31.4 |
| Adrenal gland | 0.0 | Renal ca. RXF 393 | 18.3 |
| Thyroid | 0.0 | Renal ca. ACHN | 14.3 |
| Salivary gland | 46.7 | Renal ca. UO-31 | 33.2 |
| Pituitary gland | 5.5 | Renal ca. TK-10 | 19.5 |
| Brain (fetal) | 0.0 | Liver | 4.7 |
| Brain (whole) | 29.1 | Liver (fetal) | 4.0 |
| Brain (amygdala) | 13.4 | Liver ca. | 37.4 |
| | | (hepatoblast) HepG2 | |
| Brain (cerebellum) | 3.3 | Lung | 0.0 |
| Brain (hippocampus) | 22.5 | Lung (fetal) | 9.9 |
| Brain (substantia nigra) | 18.7 | Lung ca. (small cell) | 26.4 |
| | | LX-1 | |
| Brain (thalamus) | 100.0 | Lung ca. (small cell) | 31.2 |
| | | NCI-H69 | |
| Cerebral Cortex | 19.6 | Lung ca. (s.cell var.) | 53.6 |
| | | SHP-77 | |
| Spinal cord | 29.9 | Lung ca. (large | 4.2 |
| | | cell)NCI-H460 | |
| glio/astro U87-MG | 37.1 | Lung ca. (non-sm. | 11.6 |
| | | cell) A549 | |
| glio/astro U-118-MG | 24.3 | Lung ca. (non-s.cell) | 15.5 |
| | | NCI-H23 | |
| astrocytoma SW1783 | 15.0 | Lung ca. (non-s.cell) | 24.8 |
| | | HOP-62 | |
| neuro*; met SK-N-AS | 21.5 | Lung ca. (non-s.cl) | 24.8 |
| | | NCI-H522 | |
| astrocytoma SF-539 | 26.1 | Lung ca. (squam.) | 10.7 |
| | | SW 900 | |
| astrocytoma SNB-75 | 31.6 | Lung ca. (squam.) | 30.1 |
| | | NCI-H596 | |
| glioma SNB-19 | 25.5 | Mammary gland | 12.8 |
| glioma U251 | 7.9 | Breast ca.* (pl.ef) | 17.4 |
| | | MCF-7 | |
| glioma SF-295 | 3.4 | Breast ca.* (pl.ef) | 21.8 |
| | | MDA-MB-231 | |
| Heart (fetal) | 3.3 | Breast ca.* (pl.ef) | 15.4 |
| | | T47D | |
| Heart | 0.0 | Breast ca. BT-549 | 16.5 |
| Skeletal muscle (fetal) | 3.0 | Breast ca. MDA-N | 13.4 |
| Skeletal muscle | 21.2 | Ovary | 3.8 |
| Bone marrow | 2.4 | Ovarian ca. OVCAR-3 | 12.0 |
| Thymus | 22.4 | Ovarian ca. OVCAR-4 | 39.5 |
| Spleen | 4.6 | Ovarian ca. OVCAR-5 | 34.6 |
| Lymph node | 6.3 | Ovarian ca. OVCAR-8 | 2.8 |
| Colorectal | 45.1 | Ovarian ca. IGROV-1 | 12.4 |
| Stomach | 6.0 | Ovarian ca.* (ascites) | 3.7 |
| | | SK-OV-3 | |
| Small intestine | 19.6 | Uterus | 0.0 |
| Colon ca. SW480 | 60.3 | Placenta | 23.8 |
| Colon ca.* | 13.2 | Prostate | 5.4 |
| SW620(SW480 met) | | | |
| Colon ca. HT29 | 6.0 | Prostate ca.* (bone | 24.0 |
| | | met)PC-3 | |
| Colon ca. HCT-116 | 13.5 | Testis | 11.8 |
| Colon ca. CaCo-2 | 31.6 | Melanoma | 5.0 |
| | | Hs688(A).T | |
| Colon ca. | 41.2 | Melanoma* (met) | 7.6 |
| tissue(ODO3866) | | Hs688(B).T | |
| Colon ca. HCC-2998 | 32.5 | Melanoma UACC-62 | 25.7 |
| Gastric ca.* (liver met) | 11.3 | Melanoma M14 | 12.8 |
| NCI-N87 | | | |
| Bladder | 45.1 | Melanoma LOX | 6.7 |
| | | IMVI | |
| Trachea | 8.5 | Melanoma* (met) | 30.1 |
| | | SK-MEL-5 | |
| Kidney | 3.7 | Adipose | 3.2 |

TABLE ARD
Panel 4D

| | Rel. Exp. (%) | | Rel. Exp. (%) |
|---|---|---|---|
| | Ag2993, Run | | Ag2993, Run |
| Tissue Name | 164315034 | Tissue Name | 164315034 |
| Secondary Th1 act | 8.4 | HUVEC IL-1beta | 3.8 |
| Secondary Th2 act | 11.8 | HUVEC IFN gamma | 4.5 |
| Secondary Tr1 act | 14.3 | HUVEC TNF alpha + IFN | 4.2 |
| | | gamma | |
| Secondary Th1 rest | 3.3 | HUVEC TNF alpha + IL4 | 5.0 |
| Secondary Th2 rest | 6.0 | HUVEC IL-11 | 5.7 |
| Secondary Tr1 rest | 3.8 | Lung Microvascular EC | 3.8 |
| | | none | |
| Primary Th1 act | 22.7 | Lung Microvascular EC | 3.3 |
| | | TNF alpha + IL-1beta | |
| Primary Th2 act | 16.0 | Microvascular Dermal EC | 10.2 |
| | | none | |
| Primary Tr1 act | 30.4 | Microsvasular Dermal EC | 5.5 |
| | | TNF alpha + IL-1beta | |
| Primary Th1 rest | 39.0 | Bronchial epithelium | 18.0 |
| | | TNF alpha + IL1beta | |
| Primary Th2 rest | 31.9 | Small airway epithelium | 5.2 |
| | | none | |
| Primary Tr1 rest | 20.6 | Small airway epithelium | 25.9 |
| | | TNF alpha + IL-1beta | |
| CD45RA CD4 | 10.0 | Coronery artery SMC rest | 4.5 |
| lymphocyte act | | | |
| CD45RO CD4 | 24.1 | Coronery artery SMC | 1.5 |
| lymphocyte act | | TNF alpha + IL-1beta | |
| CD8 lymphocyte act | 17.4 | Astrocytes rest | 4.5 |
| Secondary CD8 | 13.0 | Astrocytes TNF alpha + | 5.0 |
| lymphocyte rest | | IL-1beta | |
| Secondary CD8 | 10.7 | KU-812 (Basophil) rest | 14.0 |
| lymphocyte act | | | |
| CD4 lymphocyte none | 9.0 | KU-812 (Basophil) | 22.4 |
| | | PMA/ionomycin | |
| 2ry Th1/Th2/Tr1_anti- | 12.5 | CCD1106 (Keratinocytes) | 8.7 |
| CD95 CH11 | | none | |
| LAK cells rest | 13.3 | CCD1106 (Keratinocytes) | 6.1 |
| | | TNF alpha + IL-1beta | |
| LAK cells IL-2 | 14.4 | Liver cirrhosis | 6.0 |
| LAK cells IL-2 + IL-12 | 13.1 | Lupus kidney | 5.0 |
| LAK cells IL-2 + IFN | 15.8 | NCI-H292 none | 25.3 |
| gamma | | | |
| LAK cells IL-2 + IL-18 | 13.8 | NCI-H292 IL-4 | 28.3 |
| LAK cells | 3.9 | NCI-H292 IL-9 | 31.2 |
| PMA/ionomycin | | | |
| NK Cells IL-2 rest | 10.7 | NCI-H292 IL-13 | 14.9 |
| Two Way MLR 3 day | 11.7 | NCI-H292 IFN gamma | 11.9 |
| Two Way MLR 5 day | 6.8 | HPAEC none | 4.9 |
| Two Way MLR 7 day | 6.0 | HPAEC TNF alpha + IL-1 | 4.7 |
| | | beta | |
| PBMC rest | 3.3 | Lung fibroblast none | 2.6 |
| PBMC PWM | 30.4 | Lung fibroblast TNF alpha + | 1.4 |
| | | IL-1beta | |
| PBMC PHA-L | 20.9 | Lung fibroblast IL-4 | 7.1 |
| Ramos (B cell) none | 22.1 | Lung fibroblast IL-9 | 6.5 |
| Ramos (B cell) | 100.0 | Lung fibroblast IL-13 | 4.9 |
| ionomycin | | | |
| B lymphocytes PWM | 51.4 | Lung fibroblast IFN | 7.1 |
| | | gamma | |
| B lymphocytes CD40L | 24.8 | Dermal fibroblast | 9.7 |
| and IL-4 | | CCD1070 rest | |
| EOL-1 dbcAMP | 5.6 | Dermal fibroblast | 24.1 |
| | | CCD1070 TNF alpha | |
| EOL-1 dbcAMP | 4.5 | Dermal fibroblast | 3.9 |
| PMA/ionomycin | | CCD1070 IL-1beta | |
| Dendritic cells none | 7.0 | Dermal fibroblast IFN | 4.0 |
| | | gamma | |
| Dendritic cells LPS | 5.7 | Dermal fibroblast IL-4 | 5.9 |
| Dendritic cells anti- | 8.7 | IBD Colitis 2 | 0.2 |
| CD40 | | | |
| Monocytes rest | 3.0 | IBD Crohn's | 0.0 |
| Monocytes LPS | 1.6 | Colon | 8.2 |
| Macrophages rest | 11.3 | Lung | 7.3 |
| Macrophages LPS | 5.8 | Thymus | 27.0 |
| HUVEC none | 6.7 | Kidney | 23.0 |
| HUVEC starved | 15.6 | | |

CNS_neurodegeneration_v1.0 Summary: Ag2993 This panel does not show differential expression of the NOV47 gene in Alzheimer's disease. However, this expression profile confirms the presence of this gene in the brain. Please see Panel 1.3D for discussion of utility of this gene in the central nervous system.

Panel 1.3D Summary: Ag2993 Significant expression of the NOV47 gene is restricted to the brain, with expression in the thalamus (CT=34.4). This gene also shows strong expression in the brain in the previous panel suggesting that this gene product may be involved in the normal functioning of the brain. Thus, the protein encoded by this gene may represent a small molecule target for the treatment of neurologic diseases.

Panel 4D Summary: Ag2993 The NOV47 gene, a Peptidyl Prolyl Cis-Trans Isomerase A homolog, is a novel member of the family of receptors for the widely used immunosuppressants cyclosporin A and FK506 (see Wang et al., 2001). The NOV47 gene is expressed at moderate levels in many of the tissues in this panel and is expressed at a somewhat higher level (CT=30.3) in ionomycin-stimulated Ramos B lymphocytes. Therefore, small molecule drugs that antagonzie the activity of the NOV47 gene product may be useful as immunosuppressants to reduce or eliminate the symptoms in patients with autoimmune or inflammatory conditions, such as Crohn's disease, ulcerative colitis, multiple sclerosis, chronic obstructive pulmonary disease, asthma, emphysema, rheumatoid arthritis, lupus erythematosus, or psoriasis.

References:

Wang H C, Kim K, Bakhtiar R, Germanas J P. Structure-activity studies of ground- and transition-state analogue inhibitors of cyclophilin. J Med Chem Aug. 2, 2001;44(16):2593-600

Peptidyl-prolyl isomerases (PPIases) are ubiquitous cellular enzymes that play roles in cellular signaling and protein folding. In addition, these proteins are the receptors for the widely used immunosuppressants cyclosporin A and FK506. We report the first structure-activity studies of de novo designed inhibitors of cyclophilin, the cellular target of cyclosporin A. Our mechanism-based inhibitors were modeled on the ground- and transition-state structures of proline-containing peptides, the natural substrates of the enzyme. Both ground-state analogues 1 and transition-state analogues 2 were prepared as single enantiomers from L-proline following a “self-reproduction of chirality” procedure. The binding affinities of the analogues for the active site of cyclophilin were measured by a fluorescence perturbation assay. While the transition-state analogues 2 did not display significant avidity for the active site (K(d)=77 microM for 2b), several ground-state analogues bound to the enzyme with low micromolar affinity (K(d)=1.5 microM for 1e). These results proclaim that properly designed small molecular weight molecules can form strong complexes with cyclophilin and may find use as probes in cell biology and as therapeutic agents.

NOV48a

Expression of gene NOV48a was assessed using the primer-probe set Ag3006, described in Table ASA. Results of the RTQ-PCR runs are shown in Tables ASB, ASC, ASD, ASE and ASF.

TABLE ASA
Probe Name Ag3006

| | | | Start | |
|---|---|---|---|---|
| Primers | Sequences | Length | Positopn | SEQ ID NO: |
| Forward | 5′-gggcttggaatagagaaacct-3′ | 21 | 2991 | 1189 |
| Probe | TET-5′-caacttcctcaaagcccaaagccaag-3′-TAMRA | 26 | 3037 | 1190 |
| Reverse | 5′-gaagccttgagccttgatttat-3′ | 22 | 3069 | 1191 |

TABLE ASB
AI_comprehensive panel_v1.0

| | Rel. Exp. (%) | | Rel. Exp. (%) |
|---|---|---|---|
| | Ag3006, Run | | Ag3006, |
| Tissue Name | 211059882 | Tissue Name | Run 211059882 |
| 110967 COPD-F | 27.2 | 112427 Match Control | 72.7 |
| | | Psoriasis-F | |
| 110980 COPD-F | 18.8 | 112418 Psoriasis-M | 20.2 |
| 110968 COPD-M | 20.0 | 112723 Match Control | 19.8 |
| | | Psoriasis-M | |
| 110977 COPD-M | 71.7 | 112419 Psoriasis-M | 32.8 |
| 110989 Emphysema-F | 46.0 | 112424 Match Control | 22.7 |
| | | Psoriasis-M | |
| 110992 Emphysema-F | 18.3 | 112420 Psoriasis-M | 74.2 |
| 110993 Emphysema-F | 23.8 | 112425 Match Control | 56.6 |
| | | Psoriasis-M | |
| 110994 Emphysema-F | 20.7 | 104689 (MF) OA | 55.1 |
| | | Bone-Backus | |
| 110995 Emphysema-F | 19.9 | 104690 (MF) Adj | 70.7 |
| | | “Normal” Bone-Backus | |
| 110996 Emphysema-F | 4.3 | 104691 (MF) OA | 42.0 |
| | | Synovium-Backus | |
| 110997 Asthma-M | 6.4 | 104692 (BA) OA | 50.7 |
| | | Cartilage-Backus | |
| 111001 Asthma-F | 33.4 | 104694 (BA) OA | 32.5 |
| | | Bone-Backus | |
| 111002 Asthma-F | 33.0 | 104695 (BA) Adj | 52.1 |
| | | “Normal” Bone-Backus | |
| 111003 Atopic | 31.6 | 104696 (BA) OA | 25.7 |
| Asthma-F | | Synovium-Backus | |
| 111004 Atopic | 28.5 | 104700 (SS) OA Bone- | 10.4 |
| Asthma-F | | Backus | |
| 111005 Atopic | 11.1 | 104701 (SS) Adj | 42.0 |
| Asthma-F | | “Normal” Bone-Backus | |
| 111006 Atopic | 5.8 | 104702 (SS) OA | 71.2 |
| Asthma-F | | Synovium-Backus | |
| 111417 Allergy-M | 13.7 | 117093 OA Cartilage | 23.8 |
| | | Rep7 | |
| 112347 Allergy-M | 6.2 | 112672 OA Bone5 | 57.8 |
| 112349 Normal Lung-F | 3.8 | 112673 OA Synovium5 | 22.7 |
| 112357 Normal Lung-F | 39.8 | 112674 OA Synovial | 27.5 |
| | | Fluid cells5 | |
| 112354 Normal Lung-M | 22.1 | 117100 OA Cartilage | 12.3 |
| | | Rep14 | |
| 112374 Crohns-F | 36.9 | 112756 OA Bone9 | 41.2 |
| 112389 Match | 15.6 | 112757 OA Synovium9 | 100.0 |
| Control Crohns-F | | | |
| 112375 Crohns-F | 20.6 | 112758 OA Synovial | 19.2 |
| | | Fluid Cells9 | |
| 112732 Match | 17.9 | 117125 RA Cartilage | 25.0 |
| Control Crohns-F | | Rep2 | |
| 112725 Crohns-M | 6.1 | 113492 Bone2 RA | 19.8 |
| 112387 Match | 14.9 | 113493 Synovium2 RA | 7.7 |
| Control Crohns-M | | | |
| 112378 Crohns-M | 4.8 | 113494 Syn Fluid Cells | 14.5 |
| | | RA | |
| 112390 Match | 48.0 | 113499 Cartilage4 RA | 24.1 |
| Control Crohns-M | | | |
| 112726 Crohns-M | 33.0 | 113500 Bone4 RA | 25.9 |
| 112731 Match | 25.2 | 113501 Synovium4 RA | 15.4 |
| Control Crohns-M | | | |
| 112380 Ulcer Col-F | 32.3 | 113502 Syn Fluid | 7.5 |
| | | Cells4 RA | |
| 112734 Match | 32.5 | 113495 Cartilage3 RA | 11.8 |
| Control Ulcer Col-F | | | |
| 112384 Ulcer Col-F | 35.6 | 113496 Bone3 RA | 12.6 |
| 112737 Match | 6.5 | 113497 Synovium3 RA | 5.6 |
| Control Ulcer Col-F | | | |
| 112386 Ulcer Col-F | 17.3 | 113498 Syn Fluid | 13.9 |
| | | Cells3 RA | |
| 112738 Match | 11.3 | 117106 Normal | 14.2 |
| Control Ulcer Col-F | | Cartilage Rep20 | |
| 112381 Ulcer Col-M | 5.1 | 113663 Bone3 Normal | 6.8 |
| 112735 Match | 41.2 | 113664 Synovium3 | 1.1 |
| Control Ulcer Col-M | | Normal | |
| 112382 Ulcer Col-M | 25.2 | 113665 Syn Fluid | 3.8 |
| | | Cells3 Normal | |
| 112394 Match | 11.2 | 117107 Normal | 15.0 |
| Control Ulcer Col-M | | Cartilage Rep22 | |
| 112383 Ulcer Col-M | 23.0 | 113667 Bone4 Normal | 23.8 |
| 112736 Match | 13.0 | 113668 Synovium4 | 24.5 |
| Control Ulcer Col-M | | Normal | |
| 112423 Psoriasis-F | 34.9 | 113669 Syn Fluid | 37.9 |
| | | Cells4 Normal | |

TABLE ASC
Panel 1.3D

| | Rel. Exp. (%) | | Rel. Exp. (%) |
|---|---|---|---|
| | Ag3006, | | Ag3006, |
| Tissue Name | Run 165517770 | Tissue Name | Run 165517770 |
| Liver adenocarcinoma | 4.7 | Kidney (fetal) | 1.2 |
| Pancreas | 0.7 | Renal ca. 786-0 | 0.8 |
| Pancreatic ca. CAPAN 2 | 1.1 | Renal ca. A498 | 2.5 |
| Adrenal gland | 1.0 | Renal ca. RXF 393 | 0.9 |
| Thyroid | 1.9 | Renal ca. ACHN | 2.2 |
| Salivary gland | 2.0 | Renal ca. UO-31 | 1.7 |
| Pituitary gland | 7.0 | Renal ca. TK-10 | 1.7 |
| Brain (fetal) | 2.2 | Liver | 0.2 |
| Brain (whole) | 4.0 | Liver (fetal) | 0.7 |
| Brain (amygdala) | 4.6 | Liver ca. | 1.8 |
| | | (hepatoblast) HepG2 | |
| Brain (cerebellum) | 2.1 | Lung | 1.2 |
| Brain (hippocampus) | 3.6 | Lung (fetal) | 0.9 |
| Brain (substantia nigra) | 3.5 | Lung ca. (small cell) | 3.0 |
| | | LX-1 | |
| Brain (thalamus) | 8.0 | Lung ca. (small cell) | 0.6 |
| | | NCI-H69 | |
| Cerebral Cortex | 2.3 | Lung ca. (s. cell var.) | 2.4 |
| | | SHP-77 | |
| Spinal cord | 5.2 | Lung ca. (large | 6.1 |
| | | cell)NCI-H460 | |
| glio/astro U87-MG | 1.2 | Lung ca. (non-sm. | 0.9 |
| | | cell) A549 | |
| glio/astro U-118-MG | 9.9 | Lung ca. (non-s. cell) | 2.8 |
| | | NCI-H23 | |
| astrocytoma SW1783 | 1.2 | Lung ca. (non-s. cell) | 1.1 |
| | | HOP-62 | |
| neuro*; met SK-N-AS | 8.8 | Lung ca. (non-s. cl) | 2.1 |
| | | NCI-H522 | |
| astrocytoma SF-539 | 2.6 | Lung ca. (squam.) | 1.3 |
| | | SW 900 | |
| astrocytoma SNB-75 | 3.7 | Lung ca. (squam.) | 2.6 |
| | | NCI-H596 | |
| glioma SNB-19 | 2.7 | Mammary gland | 2.1 |
| glioma U251 | 15.3 | Breast ca.* (pl. ef) | 0.8 |
| | | MCF-7 | |
| glioma SF-295 | 2.2 | Breast ca.* (pl. ef) | 3.1 |
| | | MDA-MB-231 | |
| Heart (fetal) | 0.2 | Breast ca.* (pl. ef) | 0.7 |
| | | T47D | |
| Heart | 1.8 | Breast ca. BT-549 | 2.7 |
| Skeletal muscle (fetal) | 4.4 | Breast ca. MDA-N | 1.6 |
| Skeletal muscle | 100.0 | Ovary | 1.2 |
| Bone marrow | 1.8 | Ovarian ca. OVCAR-3 | 1.7 |
| Thymus | 0.6 | Ovarian ca. OVCAR-4 | 2.2 |
| Spleen | 2.5 | Ovarian ca. OVCAR-5 | 1.7 |
| Lymph node | 4.7 | Ovarian ca. OVCAR-8 | 0.6 |
| Colorectal | 2.9 | Ovarian ca. IGROV-1 | 0.9 |
| Stomach | 3.2 | Ovarian ca.* (ascites) | 2.0 |
| | | SK-OV-3 | |
| Small intestine | 9.9 | Uterus | 3.2 |
| Colon ca. SW480 | 0.9 | Placenta | 0.8 |
| Colon ca.* | 1.3 | Prostate | 4.2 |
| SW620(SW480 met) | | | |
| Colon ca. HT29 | 0.2 | Prostate ca.* (bone | 1.6 |
| | | met)PC-3 | |
| Colon ca. HCT-116 | 2.0 | Testis | 6.3 |
| Colon ca. CaCo-2 | 1.2 | Melanoma | 0.9 |
| | | Hs688(A).T | |
| Colon ca. | 0.9 | Melanoma* (met) | 0.3 |
| tissue(ODO3866) | | Hs688(B).T | |
| Colon ca. HCC-2998 | 2.2 | Melanoma UACC-62 | 3.1 |
| Gastric ca.* (liver met) | 3.9 | Melanoma M14 | 5.0 |
| NCI-N87 | | | |
| Bladder | 0.8 | Melanoma LOX | 0.7 |
| | | IMVI | |
| Trachea | 2.3 | Melanoma* (met) | 0.8 |
| | | SK-MEL-5 | |
| Kidney | 4.0 | Adipose | 1.1 |

TABLE ASD
Panel 2D

| | Rel. Exp. (%) | | Rel. Exp. (%) |
|---|---|---|---|
| | Ag3006, Run | | Ag3006, |
| Tissue Name | 163577592 | Tissue Name | Run 163577592 |
| Normal Colon | 35.8 | Kidney Margin | 9.0 |
| | | 8120608 | |
| CC Well to Mod Diff | 5.6 | Kidney Cancer | 13.2 |
| (ODO3866) | | 8120613 | |
| CC Margin (ODO3866) | 6.2 | Kidney Margin | 16.2 |
| | | 8120614 | |
| CC Gr.2 rectosigmoid | 5.5 | Kidney Cancer | 8.7 |
| (ODO3868) | | 9010320 | |
| CC Margin (ODO3868) | 10.8 | Kidney Margin | 20.9 |
| | | 9010321 | |
| CC Mod Diff (ODO3920) | 9.5 | Normal Uterus | 6.3 |
| CC Margin (ODO3920) | 19.6 | Uterus Cancer 064011 | 9.4 |
| CC Gr.2 ascend colon | 10.7 | Normal Thyroid | 21.0 |
| (ODO3921) | | | |
| CC Margin (ODO3921) | 8.5 | Thyroid Cancer | 2.5 |
| | | 064010 | |
| CC from Partial | 6.1 | Thyroid Cancer | 5.6 |
| Hepatectomy (ODO4309) | | A302152 | |
| Mets | | | |
| Liver Margin (ODO4309) | 0.9 | Thyroid Margin | 8.2 |
| | | A302153 | |
| Colon mets to lung | 6.8 | Normal Breast | 24.7 |
| (OD04451-01) | | | |
| Lung Margin (OD04451- | 2.8 | Breast Cancer | 65.1 |
| 02) | | (OD04566) | |
| Normal Prostate 6546-1 | 66.9 | Breast Cancer | 70.2 |
| | | (OD04590-01) | |
| Prostate Cancer | 16.6 | Breast Cancer Mets | 68.3 |
| (OD04410) | | (OD04590-03) | |
| Prostate Margin | 18.0 | Breast Cancer | 100.0 |
| (OD04410) | | Metastasis | |
| | | (OD04655-05) | |
| Prostate Cancer | 13.7 | Breast Cancer 064006 | 14.7 |
| (OD04720-01) | | | |
| Prostate Margin | 25.5 | Breast Cancer 1024 | 33.7 |
| (OD04720-02) | | | |
| Normal Lung 061010 | 15.1 | Breast Cancer | 26.6 |
| | | 9100266 | |
| Lung Met to Muscle | 22.4 | Breast Margin | 15.3 |
| (ODO4286) | | 9100265 | |
| Muscle Margin | 52.5 | Breast Cancer | 16.7 |
| (ODO4286) | | A209073 | |
| Lung Malignant Cancer | 9.9 | Breast Margin | 16.3 |
| (OD03126) | | A2090734 | |
| Lung Margin (OD03126) | 11.2 | Normal Liver | 6.8 |
| Lung Cancer (OD04404) | 3.9 | Liver Cancer 064003 | 1.6 |
| Lung Margin (OD04404) | 8.7 | Liver Cancer 1025 | 2.1 |
| Lung Cancer (OD04565) | 6.5 | Liver Cancer 1026 | 2.0 |
| Lung Margin (OD04565) | 6.2 | Liver Cancer 6004-T | 2.3 |
| Lung Cancer (OD04237- | 13.3 | Liver Tissue 6004-N | 3.2 |
| 01) | | | |
| Lung Margin (OD04237- | 11.4 | Liver Cancer 6005-T | 2.6 |
| 02) | | | |
| Ocular Mel Met to Liver | 5.2 | Liver Tissue 6005-N | 0.6 |
| (ODO4310) | | | |
| Liver Margin (ODO4310) | 2.0 | Normal Bladder | 9.7 |
| Melanoma Mets to Lung | 6.3 | Bladder Cancer 1023 | 2.2 |
| (OD04321) | | | |
| Lung Margin (OD04321) | 11.5 | Bladder Cancer | 4.4 |
| | | A302173 | |
| Normal Kidney | 47.6 | Bladder Cancer | 10.3 |
| | | (OD04718-01) | |
| Kidney Ca, Nuclear grade | 27.7 | Bladder Normal | 62.0 |
| 2 (OD04338) | | Adjacent (OD04718- | |
| | | 03) | |
| Kidney Margin | 43.8 | Normal Ovary | 5.0 |
| (OD04338) | | | |
| Kidney Ca Nuclear grade | 12.4 | Ovarian Cancer | 17.9 |
| 1/2 (OD04339) | | 064008 | |
| Kidney Margin | 46.7 | Ovarian Cancer | 20.3 |
| (OD04339) | | (OD04768-07) | |
| Kidney Ca, Clear cell | 20.2 | Ovary Margin | 3.6 |
| type (OD04340) | | (OD04768-08) | |
| Kidney Margin | 40.6 | Normal Stomach | 32.5 |
| (OD04340) | | | |
| Kidney Ca, Nuclear grade | 4.0 | Gastric Cancer | 9.6 |
| 3 (OD04348) | | 9060358 | |
| Kidney Margin | 30.1 | Stomach Margin | 8.6 |
| (OD04348) | | 9060359 | |
| Kidney Cancer | 9.1 | Gastric Cancer | 12.0 |
| (OD04622-01) | | 9060395 | |
| Kidney Margin | 5.6 | Stomach Margin | 14.3 |
| (OD04622-03) | | 9060394 | |
| Kidney Cancer | 9.1 | Gastric Cancer | 17.6 |
| (OD04450-01) | | 9060397 | |
| Kidney Margin | 24.0 | Stomach Margin | 2.6 |
| (OD04450-03) | | 9060396 | |
| Kidney Cancer 8120607 | 18.3 | Gastric Cancer | 20.7 |
| | | 064005 | |

TABLE ASE
Panel 3D

| | Rel. Exp. (%) | | Rel. Exp. (%) |
|---|---|---|---|
| | Ag3006, Run | | Ag3006, Run |
| Tissue Name | 170188143 | Tissue Name | 170188143 |
| Daoy-Medulloblastoma | 8.5 | Ca Ski-Cervical epidermoid | 15.7 |
| | | carcinoma (metastasis) | |
| TE671-Medulloblastoma | 7.5 | ES-2-Ovarian clear cell | 8.3 |
| | | carcinoma | |
| D283 Med- | 31.6 | Ramos-Stimulated with | 6.3 |
| Medulloblastoma | | PMA/ionomycin 6 h | |
| PFSK-1-Primitive | 21.8 | Ramos-Stimulated with | 12.6 |
| Neuroectodermal | | PMA/ionomycin 14 h | |
| XF-498-CNS | 5.4 | MEG-01-Chronic | 3.5 |
| | | myelogenous leukemia | |
| | | (megokaryoblast) | |
| SNB-78-Glioma | 2.5 | Raji-Burkitt's lymphoma | 5.8 |
| SF-268-Glioblastoma | 13.4 | Daudi-Burkitt's lymphoma | 15.4 |
| T98G-Glioblastoma | 24.5 | U266-B-cell plasmacytoma | 11.4 |
| SK-N-SH- | 14.4 | CA46-Burkitt's lymphoma | 2.4 |
| Neuroblastoma | | | |
| (metastasis) | | | |
| SF-295-Glioblastoma | 8.0 | RL-non-Hodgkin's B-cell | 1.3 |
| | | lymphoma | |
| Cerebellum | 5.3 | JM1-pre-B-cell lymphoma | 6.4 |
| Cerebellum | 3.2 | Jurkat-T cell leukemia | 3.8 |
| NCI-H292- | 13.5 | TF-1-Erythroleukemia | 8.0 |
| Mucoepidermoid lung | | | |
| carcinoma | | | |
| DMS-114-Small cell | 9.1 | HUT 78-T-cell lymphoma | 7.8 |
| lung cancer | | | |
| DMS-79-Small cell lung | 100.0 | U937-Histiocytic lymphoma | 8.3 |
| cancer | | | |
| NCI-H146-Small cell | 6.3 | KU-812-Myelogenous | 5.8 |
| lung cancer | | leukemia | |
| NCI-H526-Small cell | 20.6 | 769-P-Clear cell renal | 7.7 |
| lung cancer | | carcinoma | |
| NCI-N417-Small cell | 21.5 | Caki-2-Clear cell renal | 13.9 |
| lung cancer | | carcinoma | |
| NCI-H82-Small cell | 15.2 | SW 839-Clear cell renal | 0.9 |
| lung cancer | | carcinoma | |
| NCI-H157-Squamous | 18.2 | G401-Wilms' tumor | 3.7 |
| cell lung cancer | | | |
| (metastasis) | | | |
| NCI-H1155-Large cell | 18.6 | Hs766T-Pancreatic | 10.5 |
| lung cancer | | carcinoma (LN metastasis) | |
| NCI-H1299-Large cell | 19.2 | CAPAN-1-Pancreatic | 6.3 |
| lung cancer | | adenocarcinoma (liver | |
| | | metastasis) | |
| NCI-H727-Lung | 7.2 | SU86.86-Pancreatic | 10.3 |
| carcinoid | | carcinoma (liver metastasis) | |
| NCI-UMC-11-Lung | 12.5 | BxPC-3-Pancreatic | 3.7 |
| carcinoid | | adenocarcinoma | |
| LX-1-Small cell lung | 10.9 | HPAC-Pancreatic | 11.0 |
| cancer | | adenocarcinoma | |
| Colo-205-Colon cancer | 7.1 | MIA PaCa-2-Pancreatic | 1.4 |
| | | carcinoma | |
| KM12-Colon cancer | 13.1 | CFPAC-1-Pancreatic ductal | 23.2 |
| | | adenocarcinoma | |
| KM20L2-Colon cancer | 1.1 | PANC-1-Pancreatic | 4.7 |
| | | epithelioid ductal carcinoma | |
| NCI-H716-Colon cancer | 7.2 | T24-Bladder carcinma | 3.5 |
| | | (transitional cell) | |
| SW-48-Colon | 4.4 | 5637-Bladder carcinoma | 4.7 |
| adenocarcinoma | | | |
| SW1116-Colon | 5.8 | HT-1197-Bladder carcinoma | 1.5 |
| adenocarcinoma | | | |
| LS 174T-Colon | 3.7 | UM-UC-3-Bladder carcinma | 4.8 |
| adenocarcinoma | | (transitional cell) | |
| SW-948-Colon | 0.5 | A204-Rhabdomyosarcoma | 35.4 |
| adenocarcinoma | | | |
| SW-480-Colon | 1.7 | HT-1080-Fibrosarcoma | 9.0 |
| adenocarcinoma | | | |
| NCI-SNU-5-Gastric | 3.1 | MG-63-Osteosarcoma | 4.3 |
| carcinoma | | | |
| KATO III-Gastric | 24.8 | SK-LMS-1-Leiomyosarcoma | 13.8 |
| carcinoma | | (vulva) | |
| NCI-SNU-16-Gastric | 6.9 | SJRH30-Rhabdomyosarcoma | 7.3 |
| carcinoma | | (met to bone marrow) | |
| NCI-SNU-1-Gastric | 18.0 | A431-Epidermoid carcinoma | 2.8 |
| carcinoma | | | |
| RF-1-Gastric | 4.1 | WM266-4-Melanoma | 5.4 |
| adenocarcinoma | | | |
| RF-48-Gastric | 3.4 | DU 145-Prostate carcinoma | 0.0 |
| adenocarcinoma | | (brain metastasis) | |
| MKN-45-Gastric | 10.0 | MDA-MB-468-Breast | 14.5 |
| carcinoma | | adenocarcinoma | |
| NCI-N87-Gastric | 6.1 | SCC-4-Squamous cell | 0.0 |
| carcinoma | | carcinoma of tongue | |
| OVCAR-5-Ovarian | 0.0 | SCC-9-Squamous cell | 0.0 |
| carcinoma | | carcinoma of tongue | |
| RL95-2-Uterine | 3.5 | SCC-15-Squamous cell | 0.3 |
| carcinoma | | carcinoma of tongue | |
| HelaS3-Cervical | 6.7 | CAL 27-Squamous cell | 5.2 |
| adenocarcinoma | | carcinoma of tongue | |

TABLE ASF
Panel 4D

| | Rel. Exp. (%) | | Rel. Exp. (%) |
|---|---|---|---|
| | Ag3006, Run | | Ag3006, Run |
| Tissue Name | 168033497 | Tissue Name | 168033497 |
| Secondary Th1 act | 32.3 | HUVEC IL-1beta | 9.0 |
| Secondary Th2 act | 25.5 | HUVEC IFN gamma | 21.9 |
| Secondary Tr1 act | 39.0 | HUVEC TNF alpha + IFN | 29.9 |
| | | gamma | |
| Secondary Th1 rest | 5.8 | HUVEC TNF alpha + IL4 | 26.4 |
| Secondary Th2 rest | 7.8 | HUVEC IL-11 | 11.0 |
| Secondary Tr1 rest | 15.9 | Lung Microvascular EC | 27.7 |
| | | none | |
| Primary Th1 act | 38.2 | Lung Microvascular EC | 28.3 |
| | | TNF alpha + IL-1beta | |
| Primary Th2 act | 33.7 | Microvascular Dermal EC | 30.8 |
| | | none | |
| Primary Tr1 act | 47.6 | Microsvasular Dermal EC | 17.4 |
| | | TNF alpha + IL-1beta | |
| Primary Th1 rest | 35.8 | Bronchial epithelium | 23.3 |
| | | TNF alpha + IL1beta | |
| Primary Th2 rest | 25.7 | Small airway epithelium | 12.3 |
| | | none | |
| Primary Tr1 rest | 23.7 | Small airway epithelium | 44.4 |
| | | TNF alpha + IL-1beta | |
| CD45RA CD4 | 23.7 | Coronery artery SMC rest | 13.2 |
| lymphocyte act | | | |
| CD45RO CD4 | 29.5 | Coronery artery SMC | 10.4 |
| lymphocyte act | | TNF alpha + IL-1beta | |
| CD8 lymphocyte act | 33.7 | Astrocytes rest | 11.5 |
| Secondary CD8 | 37.6 | Astrocytes TNF alpha + | 6.0 |
| lymphocyte rest | | IL-1beta | |
| Secondary CD8 | 16.4 | KU-812 (Basophil) rest | 14.5 |
| lymphocyte act | | | |
| CD4 lymphocyte none | 4.8 | KU-812 (Basophil) | 22.1 |
| | | PMA/ionomycin | |
| 2ry Th1/Th2/Tr1_anti- | 15.3 | CCD1106 (Keratinocytes) | 28.1 |
| CD95 CH11 | | none | |
| LAK cells rest | 11.2 | CCD1106 (Keratinocytes) | 14.8 |
| | | TNF alpha + IL-1beta | |
| LAK cells IL-2 | 29.9 | Liver cirrhosis | 2.2 |
| LAK cells IL-2 + IL-12 | 25.3 | Lupus kidney | 5.8 |
| LAK cells IL-2 + IFN | 44.1 | NCI-H292 none | 15.4 |
| gamma | | | |
| LAK cells IL-2 + IL-18 | 35.8 | NCI-H292 IL-4 | 36.3 |
| LAK cells | 8.7 | NCI-H292 IL-9 | 22.8 |
| PMA/ionomycin | | | |
| NK Cells IL-2 rest | 23.3 | NCI-H292 IL-13 | 12.7 |
| Two Way MLR 3 day | 33.0 | NCI-H292 IFN gamma | 17.4 |
| Two Way MLR 5 day | 18.3 | HPAEC none | 22.8 |
| Two Way MLR 7 day | 17.0 | HPAEC TNF alpha + IL-1 | 15.9 |
| | | beta | |
| PBMC rest | 5.0 | Lung fibroblast none | 27.5 |
| PBMC PWM | 100.0 | Lung fibroblast | 14.3 |
| | | TNF alpha + IL-1beta | |
| PBMC PHA-L | 31.2 | Lung fibroblast IL-4 | 39.0 |
| Ramos (B cell) none | 30.4 | Lung fibroblast IL-9 | 26.2 |
| Ramos (B cell) | 66.4 | Lung fibroblast IL-13 | 27.9 |
| ionomycin | | | |
| B lymphocytes PWM | 96.6 | Lung fibroblast IFN | 35.8 |
| | | gamma | |
| B lymphocytes CD40L | 32.3 | Dermal fibroblast | 31.2 |
| and IL-4 | | CCD1070 rest | |
| EOL-1 dbcAMP | 18.7 | Dermal fibroblast | 49.3 |
| | | CCD1070 TNF alpha | |
| EOL-1 dbcAMP | 15.6 | Dermal fibroblast | 9.7 |
| PMA/ionomycin | | CCD1070 IL-1beta | |
| Dendritic cells none | 4.6 | Dermal fibroblast IFN | 9.5 |
| | | gamma | |
| Dendritic cells LPS | 3.0 | Dermal fibroblast IL-4 | 15.7 |
| Dendritic cells anti- | 4.0 | IBD Colitis 2 | 6.0 |
| CD40 | | | |
| Monocytes rest | 7.5 | IBD Crohn's | 1.4 |
| Monocytes LPS | 10.8 | Colon | 25.0 |
| Macrophages rest | 6.8 | Lung | 7.9 |
| Macrophages LPS | 3.1 | Thymus | 55.1 |
| HUVEC none | 27.9 | Kidney | 18.4 |
| HUVEC starved | 35.8 | | |

AI_comprehensive panel_v1.0 Summary: Ag3006 The NOV48a gene is a novel member of the Phospholipase C family that is expressed at moderate to low levels in numerous cell types involved in the immune response in health and disease. In addition, the NOV48a gene is expressed at higher levels (CTs range 29-32) in samples obtained from patients with several autoimmune and inflammatory diseases, particularly a subset of samples from osteoarthritic synovium and psoriasis. Therefore, small molecule drugs that antagonzie the activity of this gene product may be useful as immunosuppressants to reduce or eliminate the symptoms in patients with conditions, such as osteoarthritis, psoriasis, Crohn's disease, ulcerative colitis, multiple sclerosis, chronic obstructive pulmonary disease, asthma, emphysema, rheumatoid arthritis, or lupus erythematosus.

Panel 1.3D Summary: Ag3006 Highest expression of the NOV48a gene, a phospholipase C homolog, is seen in skeletal muscle (CT=27.6). Phosphatidylinositol-specific phospholipase C (PLC) plays an important role in receptor-mediated signal transduction. In addition to skeletal muscle, this gene is expressed in heat, liver, adipose, adrenal, thyroid, and pancreas. This widespread expression in metabolic tissues suggests that this gene product may be involved in cellular regulation of metabolism through interactions with the insulin receptor. A therapeutic modulator to this gene and/or gene-product may be useful in the treatment of metabolic diseases that affect skeletal muscle metabolism. This gene and/or gene-product may also prove useful in differentiating between fetal and adult forms of skeletal muscle tissue, since it is expressed at much higher levels in the adult (CT=27) when compared to expression in the fetal tissue (CT=32).

This gene is also expressed at a low level in almost all cancer cell lines in this panel. Hence, it is probably required for cell survival and proliferation and therefore, inhibition of this gene in cancer can probably be used as therapy.

In addition, increased expression of PLC delta has been observed in the brains of Alzheimer's disease patients, indicating a role for this class of enzyme in the disease process. Therefore, inhibitors of the NOV48A protein product, by countering this disease associated process, may have utility in treating Alzheimer's disease and other neurodegenerative disorders.

References:

Tanino H, Shimohama S, Sasaki Y, Sumida Y, Fujimoto S. Increase in phospholipase C-delta1 protein levels in aluminum-treated rat brains. Biochem Biophys Res Commun May 19, 2000;271(3):620-5

The effect of administration of aluminum to rats on the level of three phospholipase C (PLC) isozymes (beta1, gamma1, and delta1) was assessed in a variety of brain tissues. After exposure to aluminum, a statistically significant increase in malondialdehyde, an index of lipid peroxidation, was observed. In addition, there was a significant reduction in the catalytic activity of low molecular weight phosphotyrosine phosphatase, which loses its activity during oxidative stress. This suggests that oxidative stress is induced in brain tissues exposed to aluminum. The protein level of PLC-delta1, but not that of PLC-beta1 or -gamma1, was significantly increased in brains where oxidative stress had been induced. The total PLC activity in aluminum-treated rat brains was significantly higher than that in control brains. These results suggest that PLC-delta1 protein levels in brain tissues are increased by the induction of oxidative stress, giving an explanation for its up-regulation in Alzheimer's disease.

Panel 2D Summary: Ag3006 The NOV48A gene is expressed at a low to moderate level in most of the tissues on this panel. There is increased expression in ovarian and breast cancer compared to normal adjacent tissue. Thus, expression of this gene could potentially be used as a diagnostic marker for the presence of cancer. Furthermore, inhibition of this gene in ovarian and breast cancer may be useful as a therapeutic treatment. Additionally, there is increased expression in normal kidney samples compared to adjacent tumors. Thus, decreased expression of this gene could be used as a diagnostic marker for kidney cancer and therapeutic modulation of expression of this gene in tumors may be used to treat these cancers.

Panel 3D Summary: Ag3006 The NOV48a gene is expressed at a low level in almost all cancer cell lines in this panel with the highest expression in DMS-79 (CT=29.21). This ubiquitous pattern of expression suggests that this gene product may be required for cell survival and proliferation and inhibition of this gene in cancer may therefore be useful as a therapy.

Panel 4D Summary: Ag3006 The NOV48a gene is ubiquitously expressed among the samples on this panel, suggesting a role for this protein product in inflammation. Please see AI_comprehensive panel_v1.0 for further discussion of utility of this gene in inflammation. Results from a second experiment with the CG56003-01 gene are not included. The amp plot indicates that there were experimental difficulties with this run.

NOV49

Expression of gene NOV49 was assessed using the primer-probe set Ag3003, described in Table ATA. Results of the RTQ-PCR runs are shown in Table ATB.

TABLE ATA
Probe Name Ag3003

| | | | Start | |
|---|---|---|---|---|
| Primers | Sequences | Length | Position | SEQ ID NO: |
| Forward | 5′-catctcgtccaccctacgtt-3′ | 20 | 299 | 1192 |
| Probe | TET-5′-cttcagctgctgttgcactcaaggat-3′-TAMRA | 26 | 339 | 1193 |
| Reverse | 5′-ttcaggaagccatagaaactca-3′ | 22 | 366 | 1194 |

TABLE ATB
Panel 1.3D

| | Rel. Exp. (%) | | Rel. Exp. (%) |
|---|---|---|---|
| | Ag3003, | | Ag3003, |
| Tissue Name | Run 166245477 | Tissue Name | Run 166245477 |
| Liver adenocarcinoma | 0.0 | Kidney (fetal) | 0.0 |
| Pancreas | 15.2 | Renal ca. 786-0 | 0.0 |
| Pancreatic ca. CAPAN 2 | 0.0 | Renal ca. A498 | 0.0 |
| Adrenal gland | 0.0 | Renal ca. RXF 393 | 0.0 |
| Thyroid | 0.0 | Renal ca. ACHN | 0.0 |
| Salivary gland | 17.0 | Renal ca. UO-31 | 0.0 |
| Pituitary gland | 0.0 | Renal ca. TK-10 | 14.0 |
| Brain (fetal) | 0.0 | Liver | 0.0 |
| Brain (whole) | 0.0 | Liver (fetal) | 0.0 |
| Brain (amygdala) | 0.0 | Liver ca. | 0.0 |
| | | (hepatoblast) HepG2 | |
| Brain (cerebellum) | 0.0 | Lung | 0.0 |
| Brain (hippocampus) | 0.0 | Lung (fetal) | 0.0 |
| Brain (substantia nigra) | 0.0 | Lung ca. (small cell) | 0.0 |
| | | LX-1 | |
| Brain (thalamus) | 0.0 | Lung ca. (small cell) | 0.0 |
| | | NCI-H69 | |
| Cerebral Cortex | 0.0 | Lung ca. (s. cell var.) | 17.7 |
| | | SHP-77 | |
| Spinal cord | 29.9 | Lung ca. (large | 0.0 |
| | | cell)NCI-H460 | |
| glio/astro U87-MG | 0.0 | Lung ca. (non-sm. | 0.0 |
| | | cell) A549 | |
| glio/astro U-118-MG | 0.0 | Lung ca. (non-s. cell) | 0.0 |
| | | NCI-H23 | |
| astrocytoma SW1783 | 0.0 | Lung ca. (non-s. cell) | 0.0 |
| | | HOP-62 | |
| neuro*; met SK-N-AS | 0.0 | Lung ca. (non-s. cl) | 0.0 |
| | | NCI-H522 | |
| astrocytoma SF-539 | 0.0 | Lung ca. (squam.) | 15.5 |
| | | SW 900 | |
| astrocytoma SNB-75 | 0.0 | Lung ca. (squam.) | 0.0 |
| | | NCI-H596 | |
| glioma SNB-19 | 0.0 | Mammary gland | 52.1 |
| glioma U251 | 0.0 | Breast ca.* (pl. ef) | 100.0 |
| | | MCF-7 | |
| glioma SF-295 | 0.0 | Breast ca.* (pl. ef) | 0.0 |
| | | MDA-MB-231 | |
| Heart (fetal) | 0.0 | Breast ca.* (pl. ef) | 0.0 |
| | | T47D | |
| Heart | 0.0 | Breast ca.* BT-549 | 0.0 |
| Skeletal muscle (fetal) | 0.0 | Breast ca. MDA-N | 0.0 |
| Skeletal muscle | 0.0 | Ovary | 0.0 |
| Bone marrow | 0.0 | Ovarian ca. OVCAR-3 | 0.0 |
| Thymus | 0.0 | Ovarian ca. OVCAR-4 | 0.0 |
| Spleen | 0.0 | Ovarian ca. OVCAR-5 | 51.8 |
| Lymph node | 0.0 | Ovarian ca. OVCAR-8 | 0.0 |
| Colorectal | 0.0 | Ovarian ca. IGROV-1 | 0.0 |
| Stomach | 0.0 | Ovarian ca.* (ascites) | 0.0 |
| | | SK-OV-3 | |
| Small intestine | 0.0 | Uterus | 0.0 |
| Colon ca. SW480 | 0.0 | Placenta | 0.0 |
| Colon ca.* | 0.0 | Prostate | 11.2 |
| SW620(SW480 met) | | | |
| Colon ca. HT29 | 0.0 | Prostate ca.* (bone | 0.0 |
| | | met)PC-3 | |
| Colon ca. HCT-116 | 0.0 | Testis | 0.0 |
| Colon ca. CaCo-2 | 0.0 | Melanoma | 0.0 |
| | | Hs688(A).T | |
| Colon ca. | 0.0 | Melanoma* (met) | 0.0 |
| tissue(ODO3866) | | Hs688(B).T | |
| Colon ca. HCC-2998 | 0.0 | Melanoma UACC-62 | 0.0 |
| Gastric ca.* (liver met) | 31.2 | Melanoma M14 | 0.0 |
| NCI-N87 | | | |
| Bladder | 0.0 | Melanoma LOX | 0.0 |
| | | IMVI | |
| Trachea | 13.3 | Melanoma* (met) | 0.0 |
| | | SK-MEL-5 | |
| Kidney | 0.0 | Adipose | 0.0 |

Panel 1.3D Summary: Ag3003 Expression of the NOV49 gene is restricted to a sample derived from a breast cancer cell line (CT=34.7). Thus, expression of this gene could be used to differentiate between this sample and other samples on this panel and as a marker to detect the presence of breast cancer. Furthermore, therapeutic modulation of the expression or function of this gene may be effective in the treatment of breast cancer.

Panel 4D Summary: Ag3003 Expression of the NOV49 gene is low/undetectable in all samples on this panel (CTs>35). (Data not shown.)

NOV50a

Expression of gene NOV50a was assessed using the primer-probe set Ag3014, described in Table AUA.

TABLE AUA
Probe Name Ag3014

| | | | Start | |
|---|---|---|---|---|
| Primers | Sequences | Length | Position | SEQ ID NO: |
| Forward | 5′-gactgagcgttgccttttct-3′ | 20 | 1065 | 1195 |
| Probe | TET-5′-agctacctcccaaagcagcctgacct-3′-TAMRA | 26 | 1088 | 1196 |
| Reverse | 5′-acaatccctgcacaacgat-3′ | 19 | 1138 | 1197 |

CNS_neurodegeneration_v1.0 Summary: Ag3014 Expression of the NOV50a gene is low/undetectable in all samples on this panel (CTs>35). (Data not shown.) The amp plot indicates that there is a high probability of a probe failure.

Panel 1.3D Summary: Ag3014 Expression of the NOV50a gene is low/undetectable in all samples on this panel (CTs>35). (Data not shown.) The amp plot indicates that there is a high probability of a probe failure.

Panel 4D Summary: Ag3014 Expression of the NOV50a gene is low/undetectable in all samples on this panel (CTs>35). (Data not shown.) The amp plot indicates that there is a high probability of a probe failure.

NOV53

Expression of gene NOV53 was assessed using the primer-probe set Ag3008, described in Table AVA. Results of the RTQ-PCR runs are shown in Tables AVB, AVC and AVD.

TABLE AVA
Probe Name Ag3008

| | | | Start | |
|---|---|---|---|---|
| Primers | Sequences | Length | Position | SEQ ID NO: |
| Forward | 5′-cttaagctgctgcctatgaatg-3′ | 22 | 469 | 1198 |
| Probe | TET-5′-atacgggagctacagaccatcatccg-3′-TAMRA | 26 | 496 | 1199 |
| Reverse | 5′-tcacctctactggctgtcttgt-3′ | 22 | 524 | 1200 |

TABLE AVB
CNS_neurodegeneration_v1.0

| | Rel. | | Rel. |
|---|---|---|---|
| | Exp. (%) | | Exp. (%) |
| | Ag3008, | | Ag3008, |
| | Run | | Run |
| Tissue Name | 211010256 | Tissue Name | 211010256 |
| AD 1 Hippo | 14.7 | Control (Path) 3 | 6.2 |
| | | Temporal Ctx | |
| AD 2 Hippo | 30.6 | Control (Path) 4 | 35.4 |
| | | Temporal Ctx | |
| AD 3 Hippo | 6.3 | AD 1 Occipital Ctx | 22.1 |
| AD 4 Hippo | 8.0 | AD 2 Occipital Ctx | 0.0 |
| | | (Missing) | |
| AD 5 Hippo | 84.1 | AD 3 Occipital Ctx | 4.9 |
| AD 6 Hippo | 57.0 | AD 4 Occipital Ctx | 22.4 |
| Control 2 Hippo | 28.9 | AD 5 Occipital Ctx | 72.2 |
| Control 4 Hippo | 22.1 | AD 6 Occipital Ctx | 21.5 |
| Control (Path) 3 | 4.7 | Control 1 Occipital | 4.7 |
| Hippo | | Ctx | |
| AD 1 Temporal Ctx | 26.1 | Control 2 Occipital | 64.6 |
| | | Ctx | |
| AD 2 Temporal Ctx | 30.8 | Control 3 Occipital | 10.7 |
| | | Ctx | |
| AD 3 Temporal Ctx | 5.9 | Control 4 Occipital | 12.2 |
| | | Ctx | |
| AD 4 Temporal Ctx | 17.6 | Control (Path) 1 | 73.7 |
| | | Occipital Ctx | |
| AD 5 Inf Temporal | 100.0 | Control (Path) 2 | 10.5 |
| Ctx | | Occipital Ctx | |
| AD 5 Sup Temporal | 50.0 | Control (Path) 3 | 3.7 |
| Ctx | | Occipital Ctx | |
| AD 6 Inf Temporal | 57.0 | Control (Path) 4 | 9.7 |
| Ctx | | Occipital Ctx | |
| AD 6 Sup Temporal | 54.7 | Control 1 Parietal | 5.0 |
| Ctx | | Ctx | |
| Control 1 Temporal | 3.9 | Control 2 Parietal | 42.3 |
| Ctx | | Ctx | |
| Control 2 Temporal | 46.3 | Control 3 Parietal | 25.5 |
| Ctx | | Ctx | |
| Control 3 Temporal | 17.9 | Control (Path) 1 | 95.9 |
| Ctx | | Parietal Ctx | |
| Control 3 Temporal | 8.0 | Control (Path) 2 | 33.4 |
| Ctx | | Parietal Ctx | |
| Control (Path) 1 | 58.2 | Control (Path) 3 | 2.7 |
| Temporal Ctx | | Parietal Ctx | |
| Control (Path) 2 | 33.2 | Control (Path) 4 | 32.8 |
| Temporal Ctx | | Parietal Ctx | |

TABLE AVC
Panel 1.3D

| | Rel. Exp. (%) | | Rel. Exp. (%) |
|---|---|---|---|
| | Ag3008, | | Ag3008, |
| Tissue Name | Run 167927168 | Tissue Name | Run 167927168 |
| Liver adenocarcinoma | 18.9 | Kidney (fetal) | 42.6 |
| Pancreas | 7.6 | Renal ca. 786-0 | 18.4 |
| Pancreatic ca. CAPAN 2 | 9.4 | Renal ca. A498 | 11.3 |
| Adrenal gland | 10.8 | Renal ca. RXF 393 | 5.7 |
| Thyroid | 8.1 | Renal ca. ACHN | 5.2 |
| Salivary gland | 5.9 | Renal ca. UO-31 | 9.1 |
| Pituitary gland | 11.6 | Renal ca. TK-10 | 10.3 |
| Brain (fetal) | 31.9 | Liver | 6.2 |
| Brain (whole) | 30.1 | Liver (fetal) | 10.7 |
| Brain (amygdala) | 17.8 | Liver ca. | 17.3 |
| | | (hepatoblast) HepG2 | |
| Brain (cerebellum) | 25.3 | Lung | 5.5 |
| Brain (hippocampus) | 25.7 | Lung (fetal) | 10.2 |
| Brain (substantia nigra) | 24.7 | Lung ca. (small cell) | 21.0 |
| | | LX-1 | |
| Brain (thalamus) | 14.1 | Lung ca. (small cell) | 1.4 |
| | | NCI-H69 | |
| Cerebral Cortex | 26.2 | Lung ca. (s. cell var.) | 94.0 |
| | | SHP-77 | |
| Spinal cord | 10.5 | Lung ca. (large | 4.7 |
| | | cell)NCI-H460 | |
| glio/astro U87-MG | 26.2 | Lung ca. (non-sm. | 36.6 |
| | | cell) A549 | |
| glio/astro U-118-MG | 22.4 | Lung ca. (non-s. cell) | 12.7 |
| | | NCI-H23 | |
| astrocytoma SW1783 | 82.4 | Lung ca. (non-s. cell) | 19.1 |
| | | HOP-62 | |
| neuro*; met SK-N-AS | 20.7 | Lung ca. (non-s. cl) | 33.2 |
| | | NCI-H522 | |
| astrocytoma SF-539 | 30.6 | Lung ca. (squam.) | 16.0 |
| | | SW 900 | |
| astrocytoma SNB-75 | 33.0 | Lung ca. (squam.) | 1.1 |
| | | NCI-H596 | |
| glioma SNB-19 | 22.7 | Mammary gland | 13.0 |
| glioma U251 | 100.0 | Breast ca.* (pl. ef) | 21.8 |
| | | MCF-7 | |
| glioma SF-295 | 38.2 | Breast ca.* (pl. ef) | 38.7 |
| | | MDA-MB-231 | |
| Heart (fetal) | 12.5 | Breast ca.* (pl. ef) | 21.3 |
| | | T47D | |
| Heart | 14.7 | Breast ca. BT-549 | 15.6 |
| Skeletal muscle (fetal) | 10.4 | Breast ca. MDA-N | 21.6 |
| Skeletal muscle | 35.4 | Ovary | 15.3 |
| Bone marrow | 7.4 | Ovarian ca. OVCAR-3 | 11.6 |
| Thymus | 21.6 | Ovarian ca. OVCAR-4 | 10.2 |
| Spleen | 9.1 | Ovarian ca. OVCAR-5 | 12.4 |
| Lymph node | 16.8 | Ovarian ca. OVCAR-8 | 8.5 |
| Colorectal | 15.0 | Ovarian ca. IGROV-1 | 6.8 |
| Stomach | 17.8 | Ovarian ca.* (ascites) | 58.2 |
| | | SK-OV-3 | |
| Small intestine | 4.7 | Uterus | 8.1 |
| Colon ca. SW480 | 12.5 | Placenta | 1.3 |
| Colon ca.* | 85.9 | Prostate | 7.2 |
| SW620(SW480 met) | | | |
| Colon ca. HT29 | 7.0 | Prostate ca.* (bone | 22.8 |
| | | met)PC-3 | |
| Colon ca. HCT-116 | 13.3 | Testis | 0.6 |
| Colon ca. CaCo-2 | 32.8 | Melanoma | 10.1 |
| | | Hs688(A).T | |
| Colon ca. | 9.3 | Melanoma* (met) | 8.1 |
| tissue(ODO3866) | | Hs688(B).T | |
| Colon ca. HCC-2998 | 19.9 | Melanoma UACC-62 | 22.4 |
| Gastric ca.* (liver met) | 10.7 | Melanoma M14 | 6.3 |
| NCI-N87 | | | |
| Bladder | 21.6 | Melanoma LOX | 18.2 |
| | | IMVI | |
| Trachea | 9.3 | Melanoma* (met) | 12.5 |
| | | SK-MEL-5 | |
| Kidney | 31.2 | Adipose | 23.0 |

TABLE AVD
Panel 4D

| | Rel. Exp. (%) | | Rel. Exp. (%) |
|---|---|---|---|
| | Ag3008, Run | | Ag3008, Run |
| Tissue Name | 164043360 | Tissue Name | 164043360 |
| Secondary Th1 act | 21.0 | HUVEC IL-1beta | 7.5 |
| Secondary Th2 act | 23.7 | HUVEC IFN gamma | 10.7 |
| Secondary Tr1 act | 23.5 | HUVEC TNF alpha + IFN | 6.8 |
| | | gamma | |
| Secondary Th1 rest | 8.2 | HUVEC TNF alpha + IL4 | 7.5 |
| Secondary Th2 rest | 8.8 | HUVEC IL-11 | 7.7 |
| Secondary Tr1 rest | 9.1 | Lung Microvascular EC | 12.0 |
| | | none | |
| Primary Th1 act | 24.8 | Lung Microvascular EC | 8.2 |
| | | TNF alpha + IL-1beta | |
| Primary Th2 act | 21.9 | Microvascular Dermal EC | 17.9 |
| | | none | |
| Primary Tr1 act | 29.1 | Microsvasular Dermal EC | 11.0 |
| | | TNF alpha + IL-1beta | |
| Primary Th1 rest | 44.8 | Bronchial epithelium | 16.4 |
| | | TNF alpha + IL1beta | |
| Primary Th2 rest | 21.3 | Small airway epithelium | 6.0 |
| | | none | |
| Primary Tr1 rest | 20.4 | Small airway epithelium | 48.3 |
| | | TNF alpha + IL-1beta | |
| CD45RA CD4 | 15.8 | Coronery artery SMC rest | 19.9 |
| lymphocyte act | | | |
| CD45RO CD4 | 30.1 | Coronery artery SMC | 8.0 |
| lymphocyte act | | TNF alpha + IL-1beta | |
| CD8 lymphocyte act | 22.8 | Astrocytes rest | 13.4 |
| Secondary CD8 | 32.5 | Astrocytes TNF alpha + | 8.9 |
| lymphocyte rest | | IL-1beta | |
| Secondary CD8 | 15.7 | KU-812 (Basophil) rest | 9.5 |
| lymphocyte act | | | |
| CD4 lymphocyte none | 8.8 | KU-812 (Basophil) | 33.0 |
| | | PMA/ionomycin | |
| 2ry Th1/Th2/Tr1_anti- | 17.0 | CCD1106 (Keratinocytes) | 12.3 |
| CD95 CH11 | | none | |
| LAK cells rest | 21.3 | CCD1106 (Keratinocytes) | 8.6 |
| | | TNF alpha + IL-1beta | |
| LAK cells IL-2 | 19.5 | Liver cirrhosis | 2.1 |
| LAK cells IL-2 + IL-12 | 17.6 | Lupus kidney | 1.4 |
| LAK cells IL-2 + IFN | 28.9 | NCI-H292 none | 19.8 |
| gamma | | | |
| LAK cells IL-2 + IL-18 | 16.2 | NCI-H292 IL-4 | 25.9 |
| LAK cells | 23.2 | NCI-H292 IL-9 | 27.0 |
| PMA/ionomycin | | | |
| NK Cells IL-2 rest | 11.0 | NCI-H292 IL-13 | 10.7 |
| Two Way MLR 3 day | 23.2 | NCI-H292 IFN gamma | 11.8 |
| Two Way MLR 5 day | 16.3 | HPAEC none | 6.0 |
| Two Way MLR 7 day | 9.6 | HPAEC TNF alpha + IL-1 | 9.0 |
| | | beta | |
| PBMC rest | 10.7 | Lung fibroblast none | 7.5 |
| PBMC PWM | 65.1 | Lung fibroblast | 6.7 |
| | | TNF alpha + IL-1beta | |
| PBMC PHA-L | 58.6 | Lung fibroblast IL-4 | 22.1 |
| Ramos (B cell) none | 33.0 | Lung fibroblast IL-9 | 18.2 |
| Ramos (B cell) | 100.0 | Lung fibroblast IL-13 | 14.7 |
| ionomycin | | | |
| B lymphocytes PWM | 74.7 | Lung fibroblast IFN | 23.2 |
| | | gamma | |
| B lymphocytes CD40L | 23.2 | Dermal fibroblast | 18.8 |
| and IL-4 | | CCD1070 rest | |
| EOL-1 dbcAMP | 12.2 | Dermal fibroblast | 46.7 |
| | | CCD1070 TNF alpha | |
| EOL-1 dbcAMP | 13.0 | Dermal fibroblast | 8.0 |
| PMA/ionomycin | | CCD1070 IL-1beta | |
| Dendritic cells none | 16.7 | Dermal fibroblast IFN | 6.7 |
| | | gamma | |
| Dendritic cells LPS | 13.0 | Dermal fibroblast IL-4 | 12.2 |
| Dendritic cells anti- | 17.1 | IBD Colitis 2 | 2.0 |
| CD40 | | | |
| Monocytes rest | 17.2 | IBD Crohn's | 2.9 |
| Monocytes LPS | 7.5 | Colon | 15.4 |
| Macrophages rest | 24.5 | Lung | 14.8 |
| Macrophages LPS | 11.8 | Thymus | 22.2 |
| HUVEC none | 9.4 | Kidney | 19.8 |
| HUVEC starved | 18.8 | | |

CNS_neurodegeneration_v1.0 Summary: Ag3008 This panel does not show differential expression of the NOV53 gene in Alzheimer's disease. However, this expression profile confirms the presence of this gene in the brain. Please see Panel 1.3D for discussion of utility of this gene in the central nervous system.

Panel 1.3D Summary: Ag3008 Highest expression of the NOV53 gene is seen in a brain cancer cell line (CT=29). In addition, this gene has low to moderate expression in all the cancer cell lines used in this panel. Thus, expression of this gene might be used as a diagnostic marker in brain, colon, renal, lung, melanoma and ovarian cancers.

This gene encodes a homolog of uracil phosphoribosyltransferase. This gene has low to moderate expression in several endocrine/metabolically-related tissues, including; adipose, adrenal, pancreas, liver and skeletal muscle. Therefore, a therapeutic modulator to this gene and/or gene-product may prove useful in the treatment of diseases which affect the endocrine system.

In addition, this gene shows moderate to low levels in the CNS and may be a small molecule target for the treatment of neurologic diseases.

Panel 4D Summary: Ag3008 The NOV53 gene, a uracil phosphoribosyl-transferase homolog is expressed at moderate to low levels in numerous cell types involved in the immune response. Higher levels of expression are seen in activated B lymphocytes, represented by ionomycin-activated Ramos (CT=27.6), and pokeweed mitogen-activated B lymphocytes (CT=28.02). Therefore, small molecules that antagonize the function of this gene product may be useful as therapeutic drugs to reduce or eliminate the symptoms in patients with autoimmune and inflammatory diseases in which B cells play a part in the initiation or progression of the disease process, such as lupus erythematosus, Crohn's disease, ulcerative colitis, multiple sclerosis, chronic obstructive pulmonary disease, asthma, emphysema, rheumatoid arthritis, or psoriasis.

NOV54a

Expression of gene NOV54a was assessed using the primer-probe sets Ag3015 and Ag3070, described in Tables AWA and AWB. Results of the RTQ-PCR runs are shown in Tables AWC, AWD, AWE and AWF.

TABLE AWA
Probe Name Ag3015

| | | | Start | |
|---|---|---|---|---|
| Primers | Sequences | Length | Position | SEQ ID NO: |
| Forward | 5′-gtgctctcactatccacctcaa-3′ | 22 | 1515 | 1201 |
| Probe | TET-5′-cacacatccatctcaagaggaacatt-3′-TAMRA | 26 | 1537 | 1202 |
| Reverse | 5′-ccatacacttccagctctgact-3′ | 22 | 1573 | 1203 |

TABLE AWB
Probe Name Ag3070

| | | | Start | |
|---|---|---|---|---|
| Primers | Sequences | Length | Position | SEQ ID NO: |
| Forward | 5′-gtgctctcactatccacctcaa-3′ | 22 | 1515 | 1204 |
| Probe | TET-5′-cacacatccatctcaagaggaacatt-3′-TAMRA | 26 | 1537 | 1205 |
| Reverse | 5′-ccatacacttccagctctgact-3′ | 22 | 1573 | 1206 |

TABLE AWC
CNS_neurodegeneration_v1.0

| | Rel. Exp. (%) | Rel. Exp. (%) | | Rel. Exp. (%) | Rel. Exp. (%) |
|---|---|---|---|---|---|
| | Ag3015, Run | Ag3070, Run | Tissue | Ag3015, Run | Ag3070, Run |
| Tissue Name | 211010356 | 208974108 | Name | 211010356 | 208974108 |
| AD 1 Hippo | 37.1 | 22.5 | Control | 11.2 | 10.5 |
| | | | (Path) 3 | | |
| | | | Temporal | | |
| | | | Ctx | | |
| AD 2 Hippo | 56.3 | 50.0 | Control | 50.0 | 29.9 |
| | | | (Path) 4 | | |
| | | | Temporal | | |
| | | | Ctx | | |
| AD 3 Hippo | 24.8 | 22.7 | AD 1 | 15.5 | 13.6 |
| | | | Occipital | | |
| | | | Ctx | | |
| AD 4 Hippo | 15.6 | 8.5 | AD 2 | 0.0 | 0.0 |
| | | | Occipital | | |
| | | | Ctx | | |
| | | | (Missing) | | |
| AD 5 hippo | 84.7 | 54.0 | AD 3 | 17.7 | 9.4 |
| | | | Occipital | | |
| | | | Ctx | | |
| AD 6 Hippo | 100.0 | 66.4 | AD 4 | 17.3 | 11.3 |
| | | | Occipital | | |
| | | | Ctx | | |
| Control 2 | 37.4 | 32.5 | AD 5 | 29.7 | 39.2 |
| Hippo | | | Occipital | | |
| | | | Ctx | | |
| Control 4 | 33.0 | 26.1 | AD 6 | 45.4 | 13.1 |
| Hippo | | | Occipital | | |
| | | | Ctx | | |
| Control (Path) | 15.2 | 8.8 | Control 1 | 9.7 | 8.2 |
| 3 Hippo | | | Occipital | | |
| | | | Ctx | | |
| AD 1 Temporal | 31.2 | 17.4 | Control 2 | 42.0 | 30.4 |
| Ctx | | | Occipital | | |
| | | | Ctx | | |
| AD 2 Temporal | 50.3 | 38.2 | Control 3 | 15.5 | 14.5 |
| Ctx | | | Occipital | | |
| | | | Ctx | | |
| AD 3 Temporal | 17.6 | 14.8 | Control 4 | 13.8 | 9.4 |
| Ctx | | | Occipital | | |
| | | | Ctx | | |
| AD 4 Temporal | 36.1 | 23.3 | Control | 92.7 | 67.8 |
| Ctx | | | (Path) 1 | | |
| | | | Occipital | | |
| | | | Ctx | | |
| AD 5 Inf | 87.1 | 69.7 | Control | 17.7 | 11.3 |
| Temporal Ctx | | | (Path) 2 | | |
| | | | Occipital | | |
| | | | Ctx | | |
| AD 5 | 87.1 | 53.2 | Control | 7.9 | 8.9 |
| SupTemporal | | | (Path) 3 | | |
| Ctx | | | Occipital | | |
| | | | Ctx | | |
| AD 6 Inf | 60.7 | 72.7 | Control | 21.8 | 14.5 |
| Temporal Ctx | | | (Path) 4 | | |
| | | | Occipital | | |
| | | | Ctx | | |
| AD 6 Sup | 55.9 | 35.8 | Control 1 | 16.5 | 10.4 |
| Temporal Ctx | | | Parietal Ctx | | |
| Control 1 | 15.9 | 8.0 | Control 2 | 77.9 | 48.6 |
| Temporal Ctx | | | Parietal Ctx | | |
| Control 2 | 56.6 | 28.9 | Control 3 | 20.3 | 19.3 |
| Temporal Ctx | | | Parietal Ctx | | |
| Control 3 | 28.3 | 17.8 | Control | 76.3 | 100.0 |
| Temporal Ctx | | | (Path) 1 | | |
| | | | Parietal Ctx | | |
| Control 4 | 21.5 | 16.7 | Control | 36.1 | 21.2 |
| Temporal Ctx | | | (Path) 2 | | |
| | | | Parietal Ctx | | |
| Control (Path) | 82.4 | 70.2 | Control | 7.1 | 9.3 |
| 1 Temporal Ctx | | | (Path) 3 | | |
| | | | Parietal Ctx | | |
| Control (Path) | 49.7 | 38.7 | Control | 41.8 | 30.6 |
| 2 Temporal Ctx | | | (Path) 4 | | |
| | | | Parietal Ctx | | |

TABLE AWD
Panel 1.3D

| | Rel. Exp. (%) | Rel. Exp. (%) | | Rel. Exp. (%) | Rel. Exp. (%) |
|---|---|---|---|---|---|
| | Ag3015, Run | Ag3070, Run | | Ag3015, Run | Ag3070, Run |
| Tissue Name | 167927212 | 167985243 | Tissue Name | 167927212 | 167985243 |
| Liver | 23.7 | 21.3 | Kidney (fetal) | 56.3 | 64.6 |
| adenocarcinoma | | | | | |
| Pancreas | 7.4 | 7.2 | Renal ca. 786-0 | 2.3 | 2.9 |
| Pancreatic ca. | 4.5 | 2.4 | Renal ca. | 13.9 | 11.7 |
| CAPAN 2 | | | A498 | | |
| Adrenal gland | 6.5 | 4.9 | Renal ca. RXF | 14.1 | 25.7 |
| | | | 393 | | |
| Thyroid | 14.6 | 14.6 | Renal ca. | 13.9 | 19.8 |
| | | | ACHN | | |
| Salivary gland | 13.3 | 1.4 | Renal ca. UO- | 7.4 | 17.8 |
| | | | 31 | | |
| Pituitary gland | 6.0 | 8.4 | Renal ca. TK- | 12.6 | 14.0 |
| | | | 10 | | |
| Brain (fetal) | 25.3 | 14.6 | Liver | 13.8 | 2.4 |
| Brain (whole) | 21.6 | 11.1 | Liver (fetal) | 5.7 | 4.9 |
| Brain (amygdala) | 28.1 | 32.3 | Liver ca. | 3.7 | 4.3 |
| | | | (hepatoblast) | | |
| | | | HepG2 | | |
| Brain (cerebellum) | 17.9 | 17.2 | Lung | 23.7 | 7.6 |
| Brain | 16.8 | 16.5 | Lung (fetal) | 20.6 | 33.0 |
| (hippocampus) | | | | | |
| Brain (substantia | 18.9 | 30.6 | Lung ca. | 8.8 | 15.2 |
| nigra) | | | (small cell) | | |
| | | | LX-1 | | |
| Brain (thalamus) | 9.1 | 2.8 | Lung ca. | 3.5 | 10.4 |
| | | | (small cell) | | |
| | | | NCI-H69 | | |
| Cerebral Cortex | 42.6 | 36.9 | Lung ca. | 13.1 | 17.3 |
| | | | (s.cell var.) | | |
| | | | SHP-77 | | |
| Spinal cord | 18.9 | 18.4 | Lung ca. (large | 4.7 | 2.2 |
| | | | cell)NCI-H460 | | |
| glio/astro U87-MG | 19.8 | 37.6 | Lung ca. (non- | 13.1 | 10.2 |
| | | | sm. cell) A549 | | |
| glio/astro U-118- | 11.9 | 18.3 | Lung ca. (non- | 5.5 | 18.7 |
| MG | | | s.cell) NCI- | | |
| | | | H23 | | |
| astrocytoma | 5.6 | 10.9 | Lung ca. (non- | 7.5 | 9.3 |
| SW1783 | | | s.cell) HOP-62 | | |
| neuro*; met SK-N- | 3.4 | 7.1 | Lung ca. (non- | 18.8 | 34.6 |
| AS | | | s.cl) NCI- | | |
| | | | H522 | | |
| astrocytoma SF- | 6.4 | 10.4 | Lung ca. | 2.0 | 4.8 |
| 539 | | | (squam.) SW | | |
| | | | 900 | | |
| astrocytoma SNB- | 12.9 | 10.1 | Lung ca. | 7.6 | 8.2 |
| 75 | | | (squam.) NCI- | | |
| | | | H596 | | |
| glioma SNB-19 | 13.3 | 16.0 | Mammary | 19.5 | 3.8 |
| | | | gland | | |
| glioma U251 | 24.5 | 36.9 | Breast ca.* | 0.9 | 2.6 |
| | | | (pl.ef) MCF-7 | | |
| glioma SF-295 | 13.4 | 18.2 | Breast ca.* | 8.0 | 9.7 |
| | | | (pl.ef) MDA- | | |
| | | | MB-231 | | |
| Heart (fetal) | 100.0 | 100.0 | Breast ca.* | 26.8 | 40.6 |
| | | | (pl.ef) T47D | | |
| Heart | 12.4 | 14.0 | Breast ca. BT- | 5.1 | 6.1 |
| | | | 549 | | |
| Skeletal muscle | 51.1 | 67.8 | Breast ca. | 11.0 | 16.2 |
| (fetal) | | | MDA-N | | |
| Skeletal muscle | 11.4 | 14.1 | Ovary | 50.0 | 53.2 |
| Bone marrow | 28.9 | 11.1 | Ovarian ca. | 8.3 | 3.3 |
| | | | OVCAR-3 | | |
| Thymus | 53.2 | 56.3 | Ovarian ca. | 11.0 | 18.7 |
| | | | OVCAR-4 | | |
| Spleen | 24.8 | 30.6 | Ovarian ca. | 20.2 | 38.4 |
| | | | OVCAR-5 | | |
| Lymph node | 60.7 | 47.6 | Ovarian ca. | 5.0 | 8.1 |
| | | | OVCAR-8 | | |
| Colorectal | 17.1 | 15.5 | Ovarian ca. | 4.7 | 7.9 |
| | | | IGROV-1 | | |
| Stomach | 8.9 | 9.7 | Ovarian ca.* | 8.5 | 14.1 |
| | | | (ascites) SK- | | |
| | | | OV-3 | | |
| Small intestine | 11.9 | 12.8 | Uterus | 25.5 | 26.2 |
| Colon ca. SW480 | 17.9 | 16.4 | Placenta | 3.4 | 5.4 |
| Colon ca.* | 21.9 | 16.0 | Prostate | 14.3 | 13.5 |
| SW620(SW480 | | | | | |
| met) | | | | | |
| Colon ca. HT29 | 2.4 | 2.3 | Prostate ca.* | 14.6 | 28.3 |
| | | | (bone met)PC-3 | | |
| Colon ca. HCT- | 3.4 | 6.8 | Testis | 6.6 | 10.3 |
| 116 | | | | | |
| Colon ca. CaCo-2 | 11.7 | 16.0 | Melanoma | 12.9 | 12.4 |
| | | | Hs688(A).T | | |
| Colon ca. | 9.9 | 18.0 | Melanoma* | 10.7 | 16.6 |
| tissue(ODO3866) | | | (met) | | |
| | | | Hs688(B).T | | |
| Colon ca. HCC- | 15.0 | 17.8 | Melanoma | 7.3 | 10.4 |
| 2998 | | | UACC-62 | | |
| Gastric ca.* (liver | 3.1 | 4.9 | Melanoma | 3.4 | 7.4 |
| met) NCI-N87 | | | M14 | | |
| Bladder | 10.5 | 5.8 | Melanoma | 14.1 | 15.7 |
| | | | LOX IMVI | | |
| Trachea | 13.2 | 10.9 | Melanoma* | 2.5 | 2.8 |
| | | | (met) SK- | | |
| | | | MEL-5 | | |
| Kidney | 26.1 | 15.0 | Adipose | 34.2 | 33.0 |

TABLE AWE
Panel 2.2

| | Rel. Exp. (%) | | Rel. Exp. (%) |
|---|---|---|---|
| | Ag3070, Run | | Ag3070, |
| Tissue Name | 173800588 | Tissue Name | Run 173800588 |
| Normal Colon | 25.0 | Kidney Margin | 75.8 |
| | | (OD04348) | |
| Colon cancer | 74.2 | Kidney malignant | 5.0 |
| (OD06064) | | cancer (OD06204B) | |
| Colon Margin | 36.3 | Kidney normal adjacent | 11.9 |
| (OD06064) | | tissue (OD06204E) | |
| Colon cancer | 4.9 | Kidney Cancer | 44.4 |
| (OD06159) | | (OD04450-01) | |
| Colon Margin | 17.4 | Kidney Margin | 15.8 |
| (OD06159) | | (OD04450-03) | |
| Colon cancer | 1.4 | Kidney Cancer 8120613 | 4.0 |
| (OD06297-04) | | | |
| Colon Margin | 18.9 | Kidney Margin | 37.4 |
| (OD06297-015) | | 8120614 | |
| CC Gr.2 ascend colon | 6.5 | Kidney Cancer 9010320 | 19.3 |
| (ODO3921) | | | |
| CC Margin (ODO3921) | 16.5 | Kidney Margin | 16.6 |
| | | 9010321 | |
| Colon cancer metastasis | 12.7 | Kidney Cancer 8120607 | 33.2 |
| (OD06104) | | | |
| Lung Margin | 34.6 | Kidney Margin | 18.4 |
| (OD06104) | | 8120608 | |
| Colon mets to lung | 8.1 | Normal Uterus | 76.3 |
| (OD04451-01) | | | |
| Lung Margin | 52.5 | Uterine Cancer 064011 | 12.8 |
| (OD04451-02) | | | |
| Normal Prostate | 19.1 | Normal Thyroid | 11.8 |
| Prostate Cancer | 0.0 | Thyroid Cancer 064010 | 7.2 |
| (OD04410) | | | |
| Prostate Margin | 13.2 | Thyroid Cancer | 37.4 |
| (OD04410) | | A302152 | |
| Normal Ovary | 60.7 | Thyroid Margin | 9.7 |
| | | A302153 | |
| Ovarian cancer | 14.8 | Normal Breast | 100.0 |
| (OD06283-03) | | | |
| Ovarian Margin | 24.1 | Breast Cancer | 13.4 |
| (OD06283-07) | | (OD04566) | |
| Ovarian Cancer 064008 | 26.6 | Breast Cancer 1024 | 34.6 |
| Ovarian cancer | 25.2 | Breast Cancer | 13.3 |
| (OD06145) | | (OD04590-01) | |
| Ovarian Margin | 24.1 | Breast Cancer Mets | 40.6 |
| (OD06145) | | (OD04590-03) | |
| Ovarian cancer | 0.0 | Breast Cancer | 52.9 |
| (OD06455-03) | | Metastasis (OD04655- | |
| | | 05) | |
| Ovarian Margin | 22.4 | Breast Cancer 064006 | 29.3 |
| (OD06455-07) | | | |
| Normal Lung | 36.9 | Breast Cancer 9100266 | 28.7 |
| Invasive poor diff. lung | 19.1 | Breast Margin 9100265 | 35.6 |
| adeno (ODO4945-01) | | | |
| Lung Margin | 44.1 | Breast Cancer A209073 | 10.7 |
| (ODO4945-03) | | | |
| Lung Malignant Cancer | 18.0 | Breast Margin | 19.5 |
| (OD03126) | | A2090734 | |
| Lung Margin | 18.3 | Breast cancer | 30.4 |
| (OD03126) | | (OD06083) | |
| Lung Cancer | 22.5 | Breast cancer node | 59.5 |
| (OD05014A) | | metastasis (OD06083) | |
| Lung Margin | 79.6 | Normal Liver | 19.1 |
| (OD05014B) | | | |
| Lung cancer (OD06081) | 9.5 | Liver Cancer 1026 | 17.3 |
| Lung Margin | 17.7 | Liver Cancer 1025 | 17.4 |
| (OD06081) | | | |
| Lung Cancer | 4.1 | Liver Cancer 6004-T | 27.0 |
| (OD04237-01) | | | |
| Lung Margin | 54.7 | Liver Tissue 6004-N | 8.7 |
| (OD04237-02) | | | |
| Ocular Melanoma | 14.3 | Liver Cancer 6005-T | 48.6 |
| Metastasis | | | |
| Ocular Melanoma | 9.7 | Liver Tissue 6005-N | 49.3 |
| Margin (Liver) | | | |
| Melanoma Metastasis | 4.4 | Liver Cancer 064003 | 23.2 |
| Melanoma Margin | 60.3 | Normal Bladder | 18.7 |
| (Lung) | | | |
| Normal Kidney | 18.6 | Bladder Cancer 1023 | 15.3 |
| Kidney Ca, Nuclear | 47.6 | Bladder Cancer | 15.1 |
| grade 2 (OD04338) | | A302173 | |
| Kidney Margin | 26.6 | Normal Stomach | 58.2 |
| (OD04338) | | | |
| Kidney Ca Nuclear | 49.7 | Gastric Cancer 9060397 | 9.1 |
| grade 1/2 (OD04339) | | | |
| Kidney Margin | 16.4 | Stomach Margin | 33.0 |
| (OD04339) | | 9060396 | |
| Kidney Ca, Clear cell | 11.9 | Gastric Cancer 9060395 | 29.9 |
| type (OD04340) | | | |
| Kidney Margin | 19.1 | Stomach Margin | 65.5 |
| (OD04340) | | 9060394 | |
| Kidney Ca, Nuclear | 7.3 | Gastric Cancer 064005 | 19.5 |
| grade 3 (OD04348) | | | |

TABLE AWF
Panel 4D

| | Rel. | Rel. | | Rel. | Rel. |
|---|---|---|---|---|---|
| | Exp. (%) | Exp. (%) | | Exp. (%) | Exp. (%) |
| | Ag3015, | Ag3070, | | Ag3015, | Ag3070, |
| | Run | Run | | Run | Run |
| Tissue Name | 164043871 | 164525657 | Tissue Name | 164043871 | 164525657 |
| Secondary Th1 act | 35.4 | 35.4 | HUVEC IL-1beta | 2.6 | 1.2 |
| Secondary Th2 act | 27.9 | 21.6 | HUVEC IFN | 9.7 | 10.5 |
| | | | gamma | | |
| Secondary Tr1 act | 28.9 | 33.7 | HUVEC TNF | 8.1 | 8.1 |
| | | | alpha + IFN | | |
| | | | gamma | | |
| Secondary Th1 rest | 21.3 | 21.5 | HUVEC TNF | 4.8 | 7.4 |
| | | | alpha + IL4 | | |
| Secondary Th2 rest | 24.0 | 21.2 | HUVEC IL-11 | 4.9 | 5.6 |
| Secondary Tr1 rest | 26.6 | 26.6 | Lung | 6.9 | 9.2 |
| | | | Microvascular EC | | |
| | | | none | | |
| Primary Th1 act | 23.5 | 19.6 | Lung | 6.7 | 7.5 |
| | | | Microvascular EC | | |
| | | | TNF alpha + IL- | | |
| | | | 1beta | | |
| Primary Th2 act | 18.3 | 20.7 | Microvascular | 12.4 | 14.8 |
| | | | Dermal EC none | | |
| Primary Tr1 act | 23.8 | 27.5 | Microsvasular | 5.5 | 7.0 |
| | | | Dermal EC | | |
| | | | TNF alpha + IL- | | |
| | | | 1beta | | |
| Primary Th1 rest | 48.6 | 54.0 | Bronchial | 5.1 | 4.8 |
| | | | epithelium | | |
| | | | TNF alpha + | | |
| | | | IL1beta | | |
| Primary Th2 rest | 33.0 | 28.7 | Small airway | 2.0 | 2.1 |
| | | | epithelium none | | |
| Primary Tr1 rest | 26.1 | 28.1 | Small airway | 3.4 | 3.0 |
| | | | epithelium | | |
| | | | TNF alpha + IL- | | |
| | | | 1beta | | |
| CD45RA CD4 | 12.7 | 14.8 | Coronery artery | 8.4 | 7.1 |
| lymphocyte act | | | SMC rest | | |
| CD45RO CD4 | 28.9 | 32.3 | Coronery artery | 4.1 | 7.3 |
| lymphocyte act | | | SMC TNF alpha + | | |
| | | | IL-1beta | | |
| CD8 lymphocyte | 27.7 | 42.0 | Astrocytes rest | 5.3 | 7.6 |
| act | | | | | |
| Secondary CD8 | 29.1 | 35.8 | Astrocytes | 3.8 | 3.2 |
| lymphocyte rest | | | TNF alpha + IL- | | |
| | | | 1beta | | |
| Secondary CD8 | 38.2 | 41.8 | KU-812 | 4.0 | 4.5 |
| lymphocyte act | | | (Basophil) rest | | |
| CD4 lymphocyte | 0.0 | 18.0 | KU-812 | 7.1 | 10.4 |
| none | | | (Basophil) | | |
| | | | PMA/ionomycin | | |
| 2ry | 26.8 | 29.7 | CCD1106 | 8.3 | 6.8 |
| Th1/Th2/Tr1_anti- | | | (Keratinocytes) | | |
| CD95 CH11 | | | none | | |
| LAK cells rest | 50.3 | 71.2 | CCD1106 | 4.2 | 2.1 |
| | | | (Keratinocytes) | | |
| | | | TNF alpha + IL- | | |
| | | | 1beta | | |
| LAK cells IL-2 | 30.1 | 35.6 | Liver cirrhosis | 3.2 | 3.0 |
| LAK cells IL-2 + IL- | 16.6 | 29.3 | Lupus kidney | 3.1 | 3.3 |
| 12 | | | | | |
| LAK cells IL- | 43.8 | 44.8 | NCI-H292 none | 7.5 | 5.7 |
| 2 + IFN gamma | | | | | |
| LAK cells IL-2 + | 26.8 | 50.0 | NCI-H292 IL-4 | 6.5 | 8.3 |
| IL-18 | | | | | |
| LAK cells | 23.8 | 24.8 | NCI-H292 IL-9 | 6.0 | 8.2 |
| PMA/ionomycin | | | | | |
| NK Cells IL-2 rest | 31.4 | 35.1 | NCI-H292 IL-13 | 3.7 | 4.1 |
| Two Way MLR 3 | 32.1 | 50.3 | NCI-H292 IFN | 3.9 | 4.5 |
| day | | | gamma | | |
| Two Way MLR 5 | 20.7 | 26.2 | HPAEC none | 9.8 | 10.2 |
| day | | | | | |
| Two Way MLR 7 | 17.1 | 25.9 | HPAEC TNF | 3.8 | 4.5 |
| day | | | alpha + IL-1beta | | |
| PBMC rest | 16.2 | 19.1 | Lung fibroblast | 20.0 | 23.3 |
| | | | none | | |
| PBMC PWM | 55.1 | 58.6 | Lung fibroblast | 7.6 | 8.0 |
| | | | TNF alpha + IL-1 | | |
| | | | beta | | |
| PBMC PHA-L | 39.8 | 43.5 | Lung fibroblast | 19.9 | 24.7 |
| | | | IL-4 | | |
| Ramos (B cell) | 4.8 | 5.2 | Lung fibroblast | 15.6 | 19.2 |
| none | | | IL-9 | | |
| Ramos (B cell) | 18.9 | 25.3 | Lung fibroblast | 15.1 | 22.7 |
| ionomycin | | | IL-13 | | |
| B lymphocytes | 73.7 | 77.9 | Lung fibroblast | 22.8 | 20.7 |
| PWM | | | IFN gamma | | |
| B lymphocytes | 25.2 | 37.4 | Dermal fibroblast | 16.4 | 17.6 |
| CD40L and IL-4 | | | CCD1070 rest | | |
| EOL-1 dbcAMP | 11.7 | 10.5 | Dermal fibroblast | 56.6 | 67.4 |
| | | | CCD1070 TNF | | |
| | | | alpha | | |
| EOL-1 dbcAMP | 14.7 | 24.3 | Dermal fibroblast | 7.4 | 9.6 |
| PMA/ionomycin | | | CCD1070 IL-1 | | |
| | | | beta | | |
| Dendritic cells | 89.5 | 100.0 | Dermal fibroblast | 13.1 | 12.0 |
| none | | | IFN gamma | | |
| Dendritic cells LPS | 38.2 | 55.9 | Dermal fibroblast | 24.1 | 33.7 |
| | | | IL-4 | | |
| Dendritic cells anti- | 100.0 | 94.0 | IBD Colitis 2 | 0.9 | 1.4 |
| CD40 | | | | | |
| Monocytes rest | 49.3 | 72.2 | IBD Crohn's | 1.4 | 1.1 |
| Monocytes LPS | 15.4 | 14.7 | Colon | 10.0 | 10.7 |
| Macrophages rest | 80.7 | 87.1 | Lung | 16.5 | 15.0 |
| Macrophages LPS | 24.0 | 30.1 | Thymus | 10.3 | 11.0 |
| HUVEC none | 6.9 | 7.9 | Kidney | 34.6 | 50.3 |
| HUVEC starved | 12.3 | 15.3 | | | |

CNS_neurodegeneration_v1.0 Summary: Ag3015/Ag2070 This panel does not show differential expression of the NOV54a gene in Alzheimer's disease. However, this expression profile confirms the presence of this gene in the brain. Please see Panel 1.3D for discussion of utility of this gene in the central nervous system.

Panel 1.3D Summary: Ag3015/Ag2070 Two experiments with the same probe and primer set produce results that are in excellent agreement, with highest expression in fetal heart (CTs=29-30). This expression is higher than the expression seen in adult heart (CTs=32-33). Thus, expression of this gene could be used to differentiate between the two sources of this tissue. This gene is expressed in other metabolic tissues including adipose, adrenal, liver, pancreas, skeletal muscle and thyroid. This gene encodes a homolog of protein phosphatase 2C (PP2C), which has been linked to the regulation of hormone-sensitive lipase, the rate-limiting enzyme in adipose tissue lipolysis (Eur J Biochem Oct. 15, 1987;168(2):399-405). PP2C may also play a role in controlling insulin signaling. Therefore, a therapeutic modulator of this gene and/or gene-product may prove useful in the treatment of diseases affecting the endocrine system.

In addition, protein phosphatase 2C plays a role in dopamine and serotonin signaling. Specifically, PP2C counters the action of these neurotransmitters on DARPP-32. These neurotransmitter systems are the primary targets of drugs that treat schizophrenia and depression. Therefore, agents that inhibit this gene product may have utility in treating these disorders.

References:

Desdouits F, Siciliano J C, Nairn A C, Greengard P, Girault J A. Dephosphorylation of Ser-137 in DARPP-32 by protein phosphatases 2A and 2C: different roles in vitro and in striatonigral neurons. Biochem J Feb. 15, 1998;330 (Pt 1):211-6

DARPP-32 (dopamine- and cAMP-regulated phosphoprotein, Mr=32000) is highly expressed in striatonigral neurons in which its phosphorylation is regulated by several neurotransmitters including dopamine and glutamate. DARPP-32 becomes a potent inhibitor of protein phosphatase 1 when it is phosphorylated on Thr-34 by cAMP- or cGMP-dependent protein kinases. DARPP-32 is also phosphorylated on Ser-137 by protein kinase CK1 (CK1), in vitro and in vivo. This phosphorylation has an important regulatory role since it inhibits the dephosphorylation of Thr-34 by calcineurin in vitro and in striatonigral neurons. Here, we show that DARPP-32 phosphorylated by CK1 is a substrate in vitro for protein phosphatases 2A and 2C, but not protein phosphatase 1 or calcineurin. However, in substantia nigra slices, dephosphorylation of Ser-137 was markedly sensitive to decreased temperature, and not detectably affected by the presence of okadaic acid under conditions in which dephosphorylation of Thr-34 by protein phosphatase 2A was inhibited. These results suggest that, in neurons, phospho-Ser-137-DARPP-32 is dephosphorylated by protein phosphatase 2C, but not 2A. Thus, DARPP-32 appears to be a component of a regulatory cascade of phosphatases in which dephosphorylation of Ser-136 by protein phosphatase 2C facilitates dephosphorylation of Thr-34 by calcineurin, removing the cyclic nucleotide-induced inhibition of protein phosphatase 1.

Overall, expression of this gene is appears to be associated with normal tissues over cancer cell lines. Thus, expression of this gene could be used to differentiate between normal and malignant tissues and potentially as a treatment for cancer.

Panel 2.2 Summary: Ag3070 As seen in the previous panel, the NOV54a gene shows greater expression in normal tissues than in samples derived from malignant tissue. Thus, expression of this gene may be useful in distinguishing the two types of tissue.

Panel 4D Summary: Ag3015/Ag2070 Two experiments with the same probe and primer set produce results that are in excellent agreement. The NOV54a gene, a protein phosphatase 2C homolog is expressed by T lymphocytes prepared under a number of conditions at moderate levels and is expressed at higher levels in treated and untreated dendritic cells, monocytes, and macrophages. Dendritic cells and macrophages are powerful antigen-presenting cells (APC) whose function is pivotal in the initiation and maintenance of normal immune responses. Autoimmunity and inflammation may also be reduced by suppression of this function. Therefore, small molecule drugs that antagonize the function of this gene product may reduce or eliminate the symptoms in patients with several types of autoimmune and inflammatory diseases, such as lupus erythematosus, Crohn's disease, ulcerative colitis, multiple sclerosis, chronic obstructive pulmonary disease, asthma, emphysema, rheumatoid arthritis, or psoriasis.

NOV55

Expression of gene NOV55 was assessed using the primer-probe set Ag3024, described in Table AXA. Results of the RTQ-PCR runs are shown in Tables AXB, AXC, AXD, AXE, AXF, AXG and AXH.

TABLE AXA
Probe Name Ag3024

| | | | Start | |
|---|---|---|---|---|
| Primers | Sequences | Length | Position | SEQ ID NO: |
| Forward | 5′-ggtgggctgctataacttgact-3′ | 22 | 1033 | 1207 |
| Probe | TET-5′-tgaaagaaacaccatcctgttgcaga-3′-TAMRA | 26 | 1069 | 1208 |
| Reverse | 5′-tgttcttcaggttgttctttgc-3′ | 22 | 1097 | 1209 |

TABLE AXB
AI_comprehensive panel_v1.0

| | Rel. Exp. (%) | | Rel. Exp. (%) |
|---|---|---|---|
| | Ag3024, Run | | Ag3024, |
| Tissue Name | 248122026 | Tissue name | Run 248122026 |
| 110967 COPD-F | 10.6 | 112427 Match Control | 37.1 |
| | | Psoriasis-F | |
| 110980 COPD-F | 9.5 | 112418 Psoriasis-M | 6.1 |
| 110968 COPD-M | 10.8 | 112723 Match Control | 0.0 |
| | | Psoriasis-M | |
| 110977 COPD-M | 16.7 | 112419 Psoriasis-M | 16.6 |
| 110989 Emphysema-F | 44.8 | 112424 Match Control | 17.8 |
| | | Psoriasis-M | |
| 110992 Emphysema-F | 3.8 | 112420 Psoriasis-M | 100.0 |
| 110993 Emphysema-F | 2.6 | 112425 Match Control | 26.2 |
| | | Psoriasis-M | |
| 110994 Emphysema-F | 1.0 | 104689 (MF) OA | 0.0 |
| | | Bone-Backus | |
| 110995 Emphysema-F | 9.0 | 104690 (MF) Adj | 0.4 |
| | | “Normal” Bone-Backus | |
| 110996 Emphysema-F | 1.3 | 104691 (MF) OA | 3.0 |
| | | Synovium-Backus | |
| 110997 Asthma-M | 1.0 | 104692 (BA) OA | 1.0 |
| | | Cartilage-Backus | |
| 111001 Asthma-F | 33.4 | 104694 (BA) OA | 0.0 |
| | | Bone-Backus | |
| 111002 Asthma-F | 37.9 | 104695 (BA) Adj | 0.6 |
| | | “Normal” Bone-Backus | |
| 111003 Atopic | 41.5 | 104696 (BA) OA | 0.5 |
| Asthma-F | | Synovium-Backus | |
| 111004 Atopic | 21.2 | 104700 (SS) OA Bone- | 0.2 |
| Asthma-F | | Backus | |
| 111005 Atopic | 39.0 | 104701 (SS) Adj | 0.3 |
| Asthma-F | | “Normal” Bone-Backus | |
| 111006 Atopic | 8.5 | 104702 (SS) OA | 4.5 |
| Asthma-F | | Synovium-Backus | |
| 111417 Allergy-M | 26.6 | 117093 OA Cartilage | 42.0 |
| | | Rep7 | |
| 112347 Allergy-M | 8.6 | 112672 OA Bone5 | 18.4 |
| 112349 Normal Lung-F | 9.2 | 112673 OA Synovium5 | 8.0 |
| 112357 Normal Lung-F | 0.6 | 112674 OA Synovial | 2.4 |
| | | Fluid cells5 | |
| 112354 Normal Lung-M | 1.3 | 117100 OA Cartilage | 5.5 |
| | | Rep14 | |
| 112374 Crohns-F | 4.0 | 112756 OA Bone9 | 3.5 |
| 112389 Match | 1.3 | 112757 OA Synovium9 | 2.1 |
| Control Crohns-F | | | |
| 112375 Crohns-F | 4.5 | 112758 OA Synovial | 11.9 |
| | | Fluid Cells9 | |
| 112732 Match | 0.0 | 117125 RA Cartilage | 2.5 |
| Control Crohns-F | | Rep2 | |
| 112725 Crohns-M | 9.9 | 113492 Bone2 RA | 1.7 |
| 112387 Match | 15.3 | 113493 Synovium2 RA | 0.0 |
| Control Crohns-M | | | |
| 112378 Crohns-M | 9.7 | 113494 Syn Fluid Cells | 1.6 |
| | | RA | |
| 112390 Match | 23.7 | 113499 Cartilage4 RA | 0.4 |
| Control Crohns-M | | | |
| 112726 Crohns-M | 2.9 | 113500 Bone4 RA | 1.3 |
| 112731 Match | 1.4 | 113501 Synovium4 RA | 0.0 |
| Control Crohns-M | | | |
| 112380 Ulcer Col-F | 8.7 | 113502 Syn Fluid | 0.2 |
| | | Cells4 RA | |
| 112734 Match | 0.7 | 113495 Cartilage3 RA | 0.2 |
| Control Ulcer Col-F | | | |
| 112384 Ulcer Col-F | 25.3 | 113496 Bone3 RA | 0.6 |
| 112737 Match | 3.0 | 113497 Synovium3 RA | 0.9 |
| Control Ulcer Col-F | | | |
| 112386 Ulcer Col-F | 3.3 | 113498 Syn Fluid | 0.6 |
| | | Cells3 RA | |
| 112738 Match | 0.6 | 117106 Normal | 14.1 |
| Control Ulcer Col-F | | Cartilage Rep20 | |
| 112381 Ulcer Col-M | 1.8 | 113663 Bone3 Normal | 8.5 |
| 112735 Match | 32.8 | 113664 Synovium3 | 4.0 |
| Control Ulcer Col-M | | Normal | |
| 112382 Ulcer Col-M | 1.5 | 113665 Syn Fluid | 5.7 |
| | | Cells3 Normal | |
| 112394 Match | 1.8 | 117107 Normal | 13.8 |
| Control Ulcer Col-M | | Cartilage Rep22 | |
| 112383 Ulcer Col-M | 6.3 | 113667 Bone4 Normal | 17.2 |
| 112736 Match | 1.1 | 113668 Synovium4 | 14.7 |
| Control Ulcer Col-M | | Normal | |
| 112423 Psoriasis-F | 12.7 | 113669 Syn Fluid | 7.6 |
| | | Cells4 Normal | |

TABLE AXC
CNS_neurodegeneration_v1.0

| | Rel. Exp. (%) | Rel. Exp. (%) | | Rel. Exp. (%) | Rel. Exp. (%) |
|---|---|---|---|---|---|
| | Ag3024, Run | Ag3024, Run | Tissue | Ag3024, Run | Ag3024, Run |
| Tissue Name | 211011006 | 233677302 | Name | 211011006 | 233677302 |
| AD 1 Hippo | 11.7 | 15.4 | Control | 1.9 | 1.9 |
| | | | (Path) 3 | | |
| | | | Temporal | | |
| | | | Ctx | | |
| AD 2 Hippo | 23.3 | 32.3 | Control | 25.2 | 41.2 |
| | | | (Path) 4 | | |
| | | | Temporal | | |
| | | | Ctx | | |
| AD 3 Hippo | 10.5 | 13.6 | AD 1 | 7.7 | 11.7 |
| | | | Occipital | | |
| | | | Ctx | | |
| AD 4 Hippo | 6.2 | 9.9 | AD 2 | 0.0 | 0.0 |
| | | | Occipital | | |
| | | | Ctx | | |
| | | | (Missing) | | |
| AD 5 hippo | 77.9 | 80.7 | AD 3 | 2.9 | 4.5 |
| | | | Occipital | | |
| | | | Ctx | | |
| AD 6 Hippo | 46.0 | 45.7 | AD 4 | 16.6 | 27.2 |
| | | | Occipital | | |
| | | | Ctx | | |
| Control 2 | 28.7 | 53.6 | AD 5 | 10.9 | 20.3 |
| Hippo | | | Occipital | | |
| | | | Ctx | | |
| Control 4 | 2.8 | 2.4 | AD 6 | 61.1 | 51.4 |
| Hippo | | | Occipital | | |
| | | | Ctx | | |
| Control (Path) | 4.5 | 3.8 | Control 1 | 1.3 | 1.1 |
| 3 Hippo | | | Occipital | | |
| | | | Ctx | | |
| AD 1 Temporal | 4.6 | 7.0 | Control 2 | 52.1 | 72.7 |
| Ctx | | | Occipital | | |
| | | | Ctx | | |
| AD 2 Temporal | 16.8 | 26.1 | Control 3 | 9.9 | 13.1 |
| Ctx | | | Occipital | | |
| | | | Ctx | | |
| AD 3 Temporal | 2.9 | 3.8 | Control 4 | 1.6 | 1.4 |
| Ctx | | | Occipital | | |
| | | | Ctx | | |
| AD 4 Temporal | 15.8 | 19.1 | Control | 81.2 | 87.1 |
| Ctx | | | (Path) 1 | | |
| | | | Occipital | | |
| | | | Ctx | | |
| AD 5 Inf | 47.6 | 94.6 | Control | 12.2 | 16.5 |
| Temporal Ctx | | | (Path) 2 | | |
| | | | Occipital | | |
| | | | Ctx | | |
| AD 5 | 42.9 | 46.0 | Control | 0.6 | 0.6 |
| SupTemporal | | | (Path) 3 | | |
| Ctx | | | Occipital | | |
| | | | Ctx | | |
| AD 6 Inf | 22.8 | 29.9 | Control | 11.0 | 14.3 |
| Temporal Ctx | | | (Path) 4 | | |
| | | | Occipital | | |
| | | | Ctx | | |
| AD 6 Sup | 24.0 | 30.1 | Control 1 | 3.5 | 4.0 |
| Temporal Ctx | | | Parietal Ctx | | |
| Control 1 | 2.3 | 2.8 | Control 2 | 16.3 | 16.8 |
| Temporal Ctx | | | Parietal Ctx | | |
| Control 2 | 35.8 | 48.0 | Control 3 | 17.2 | 26.2 |
| Temporal Ctx | | | Parietal Ctx | | |
| Control 3 | 13.1 | 14.5 | Control | 100.0 | 100.0 |
| Temporal Ctx | | | (Path) 1 | | |
| | | | Parietal Ctx | | |
| Control 4 | 4.4 | 4.2 | Control | 29.3 | 38.7 |
| Temporal Ctx | | | (Path) 2 | | |
| | | | Parietal Ctx | | |
| Control (Path) | 56.3 | 68.8 | Control | 1.1 | 2.4 |
| 1 Temporal Ctx | | | (Path) 3 | | |
| | | | Parietal Ctx | | |
| Control (Path) | 47.6 | 54.0 | Control | 42.0 | 56.6 |
| 2 Temporal Ctx | | | (Path) 4 | | |
| | | | Parietal Ctx | | |

TABLE AXD
Panel 1.3D

| | Rel. Exp. (%) | | Rel. Exp. (%) |
|---|---|---|---|
| | Ag3024, | | Ag3024, |
| Tissue Name | Run 165517896 | Tissue Name | Run 165517896 |
| Liver adenocarcinoma | 0.4 | Kidney (fetal) | 0.2 |
| Pancreas | 0.0 | Renal ca. 786-0 | 0.0 |
| Pancreatic ca. CAPAN 2 | 0.0 | Renal ca. A498 | 2.4 |
| Adrenal gland | 2.6 | Renal ca. RXF 393 | 0.0 |
| Thyroid | 0.7 | Renal ca. ACHN | 0.0 |
| Salivary gland | 0.0 | Renal ca. UO-31 | 0.0 |
| Pituitary gland | 2.0 | Renal ca. TK-10 | 0.0 |
| Brain (fetal) | 11.0 | Liver | 0.7 |
| Brain (whole) | 54.7 | Liver (fetal) | 0.0 |
| Brain (amygdala) | 27.2 | Liver ca. | 0.0 |
| | | (hepatoblast) HepG2 | |
| Brain (cerebellum) | 12.9 | Lung | 0.0 |
| Brain (hippocampus) | 36.3 | Lung (fetal) | 0.0 |
| Brain (substantia nigra) | 5.7 | Lung ca. (small cell) | 0.0 |
| | | LX-1 | |
| Brain (thalamus) | 64.2 | Lung ca. (small cell) | 0.0 |
| | | NCI-H69 | |
| Cerebral Cortex | 100.0 | Lung ca. (s. cell var.) | 0.0 |
| | | SHP-77 | |
| Spinal cord | 3.8 | Lung ca. (large | 0.0 |
| | | cell)NCI-H460 | |
| glio/astro U87-MG | 0.0 | Lung ca.(non-sm. | 0.2 |
| | | cell) A549 | |
| glio/astro U-118-MG | 14.7 | Lung ca. (non-s. cell) | 0.0 |
| | | NCI-H23 | |
| astrocytoma SW1783 | 1.7 | Lung ca. (non-s. cell) | 0.6 |
| | | HOP-62 | |
| neuro*; met SK-N-AS | 0.0 | Lung ca. (non-s. cl) | 0.0 |
| | | NCI-H522 | |
| astrocytoma SF-539 | 0.0 | Lung ca. (squam.) | 2.1 |
| | | SW 900 | |
| astrocytoma SNB-75 | 6.0 | Lung ca. (squam.) | 0.0 |
| | | NCI-H596 | |
| glioma SNB-19 | 0.6 | Mammary gland | 0.4 |
| glioma U251 | 0.0 | Breast ca.* (pl. ef) | 11.0 |
| | | MCF-7 | |
| glioma SF-295 | 0.0 | Breast ca.* (pl. ef) | 0.9 |
| | | MDA-MB-231 | |
| Heart (fetal) | 0.0 | Breast ca.* (pl. ef) | 2.3 |
| | | T47D | |
| Heart | 0.6 | Breast ca. BT-549 | 1.0 |
| Skeletal muscle (fetal) | 1.9 | Breast ca. MDA-N | 0.0 |
| Skeletal muscle | 0.4 | Ovary | 0.0 |
| Bone marrow | 0.0 | Ovarian ca. OVCAR-3 | 0.0 |
| Thymus | 0.0 | Ovarian ca. OVCAR-4 | 0.0 |
| Spleen | 0.7 | Ovarian ca. OVCAR-5 | 0.2 |
| Lymph node | 0.0 | Ovarian ca. OVCAR-8 | 0.0 |
| Colorectal | 0.8 | Ovarian ca. IGROV-1 | 0.0 |
| Stomach | 6.9 | Ovarian ca.* (ascites) | 0.0 |
| | | SK-OV-3 | |
| Small intestine | 1.4 | Uterus | 2.2 |
| Colon ca. SW480 | 0.0 | Placenta | 0.0 |
| Colon ca.* | 0.0 | Prostate | 0.4 |
| SW620(SW480 met) | | | |
| Colon ca. HT29 | 0.0 | Prostate ca.* (bone | 0.0 |
| | | met)PC-3 | |
| Colon ca. HCT-116 | 0.0 | Testis | 1.7 |
| Colon ca. CaCo-2 | 0.0 | Melanoma | 0.0 |
| | | Hs688(A).T | |
| Colon ca. | 1.0 | Melanoma* (met) | 0.0 |
| tissue(ODO3866) | | Hs688(B).T | |
| Colon ca. HCC-2998 | 0.0 | Melanoma UACC-62 | 0.2 |
| Gastric ca.* (liver met) | 0.4 | Melanoma M14 | 0.0 |
| NCI-N87 | | | |
| Bladder | 0.7 | Melanoma LOX | 0.0 |
| | | IMVI | |
| Trachea | 2.4 | Melanoma* (met) | 0.0 |
| | | SK-MEL-5 | |
| Kidney | 0.6 | Adipose | 0.0 |

TABLE AXE
Panel 2D

| | Rel. Exp. (%) | | Rel. Exp. (%) |
|---|---|---|---|
| | Ag3024, Run | | Ag3024, |
| Tissue Name | 163577593 | Tissue Name | Run 163577593 |
| Normal Colon | 4.7 | Kidney Margin | 1.3 |
| | | 8120608 | |
| CC Well to Mod Diff | 5.0 | Kidney Cancer | 100.0 |
| (ODO3866) | | 8120613 | |
| CC Margin (ODO3866) | 3.1 | Kidney Margin | 5.3 |
| | | 8120614 | |
| CC Gr.2 rectosigmoid | 0.0 | Kidney Cancer | 0.8 |
| (ODO3868) | | 9010320 | |
| CC Margin (ODO3868) | 0.3 | Kidney Margin | 6.3 |
| | | 9010321 | |
| CC Mod Diff (ODO3920) | 0.0 | Normal Uterus | 0.7 |
| CC Margin (ODO3920) | 0.5 | Uterus Cancer 064011 | 9.4 |
| CC Gr.2 ascend colon | 3.1 | Normal Thyroid | 5.7 |
| (ODO3921) | | | |
| CC Margin (ODO3921) | 2.1 | Thyroid Cancer | 0.3 |
| | | 064010 | |
| CC from Partial | 1.8 | Thyroid Cancer | 2.0 |
| Hepatectomy (ODO4309) | | A302152 | |
| Mets | | | |
| Liver Margin (ODO4309) | 0.0 | Thyroid Margin | 23.5 |
| | | A302153 | |
| Colon mets to lung | 0.0 | Normal Breast | 7.9 |
| (OD04451-01) | | | |
| Lung Margin (OD04451- | 0.0 | Breast Cancer | 7.4 |
| 02) | | (OD04566) | |
| Normal Prostate 6546-1 | 3.7 | Breast Cancer | 0.0 |
| | | (OD04590-01) | |
| Prostate Cancer | 5.2 | Breast Cancer Mets | 3.6 |
| (OD04410) | | (OD04590-03) | |
| Prostate Margin | 5.4 | Breast Cancer | 3.8 |
| (OD04410) | | Metastasis | |
| | | (OD04655-05) | |
| Prostate Cancer | 11.9 | Breast Cancer 064006 | 7.1 |
| (OD04720-01) | | | |
| Prostate Margin | 6.4 | Breast Cancer 1024 | 6.2 |
| (OD04720-02) | | | |
| Normal Lung 061010 | 0.0 | Breast Cancer | 27.7 |
| | | 9100266 | |
| Lung Met to Muscle | 0.6 | Breast Margin | 4.3 |
| (ODO4286) | | 9100265 | |
| Muscle Margin | 1.0 | Breast Cancer | 30.8 |
| (ODO4286) | | A209073 | |
| Lung Malignant Cancer | 0.0 | Breast Margin | 3.0 |
| (OD03126) | | A2090734 | |
| Lung Margin (OD03126) | 1.6 | Normal Liver | 1.3 |
| Lung Cancer (OD04404) | 0.0 | Liver Cancer 064003 | 0.4 |
| Lung Margin (OD04404) | 1.0 | Liver Cancer 1025 | 0.0 |
| Lung Cancer (OD04565) | 0.0 | Liver Cancer 1026 | 0.0 |
| Lung Margin (OD04565) | 0.0 | Liver Cancer 6004-T | 0.0 |
| Lung Cancer (OD04237- | 8.4 | Liver Tissue 6004-N | 1.1 |
| 01) | | | |
| Lung Margin (OD04237- | 0.0 | Liver Cancer 6005-T | 0.0 |
| 02) | | | |
| Ocular Mel Met to Liver | 2.5 | Liver Tissue 6005-N | 0.0 |
| (ODO4310) | | | |
| Liver Margin (ODO4310) | 0.0 | Normal Bladder | 2.8 |
| Melanoma Mets to Lung | 0.4 | Bladder Cancer 1023 | 0.0 |
| (OD04321) | | | |
| Lung Margin (OD04321) | 0.5 | Bladder Cancer | 2.2 |
| | | A302173 | |
| Normal Kidney | 23.7 | Bladder Cancer | 0.7 |
| | | (OD04718-01) | |
| Kidney Ca, Nuclear grade | 1.2 | Bladder Normal | 0.4 |
| 2 (OD04338) | | Adjacent (OD04718- | |
| | | 03) | |
| Kidney Margin | 10.6 | Normal Ovary | 0.0 |
| (OD04338) | | | |
| Kidney Ca Nuclear grade | 0.0 | Ovarian Cancer | 1.4 |
| 1/2 (OD04339) | | 064008 | |
| Kidney Margin | 7.7 | Ovarian Cancer | 0.0 |
| (OD04339) | | (OD04768-07) | |
| Kidney Ca, Clear cell | 9.3 | Ovary Margin | 3.3 |
| type (OD04340) | | (OD04768-08) | |
| Kidney Margin | 17.2 | Normal Stomach | 17.8 |
| (OD04340) | | | |
| Kidney Ca, Nuclear grade | 2.3 | Gastric Cancer | 1.0 |
| 3 (OD04348) | | 9060358 | |
| Kidney Margin | 5.1 | Stomach Margin | 13.6 |
| (OD04348) | | 9060359 | |
| Kidney Cancer | 0.0 | Gastric Cancer | 7.5 |
| (OD04622-01) | | 9060395 | |
| Kidney Margin | 2.8 | Stomach Margin | 4.1 |
| (OD04622-03) | | 9060394 | |
| Kidney Cancer | 0.0 | Gastric Cancer | 13.4 |
| (OD04450-01) | | 9060397 | |
| Kidney Margin | 17.8 | Stomach Margin | 6.7 |
| (OD04450-03) | | 9060396 | |
| Kidney Cancer 8120607 | 1.7 | Gastric Cancer | 1.2 |
| | | 064005 | |

TABLE AXF
Panel 3D

| | Rel. Exp. (%) | | Rel. Exp. (%) |
|---|---|---|---|
| | Ag3024, Run | | Ag3024, Run |
| Tissue Name | 164886426 | Tissue Name | 164886426 |
| Daoy-Medulloblastoma | 1.6 | Ca Ski-Cervical epidermoid | 0.0 |
| | | carcinoma (metastasis) | |
| TE671-Medulloblastoma | 0.0 | ES-2-Ovarian clear cell | 0.3 |
| | | carcinoma | |
| D283 Med- | 0.0 | Ramos-Stimulated with | 0.0 |
| Medulloblastoma | | PMA/ionomycin 6 h | |
| PFSK-1-Primitive | 0.0 | Ramos-Stimulated with | 0.0 |
| Neuroectodermal | | PMA/ionomycin 14 h | |
| XF-498-CNS | 0.6 | MEG-01-Chronic | 0.6 |
| | | myelogenous leukemia | |
| | | (megokaryoblast) | |
| SNB-78-Glioma | 0.0 | Raji-Burkitt's lymphoma | 0.0 |
| SF-268-Glioblastoma | 0.4 | Daudi-Burkitt's lymphoma | 0.0 |
| T98G-Glioblastoma | 14.1 | U266-B-cell plasmacytoma | 0.0 |
| SK-N-SH- | 0.2 | CA46-Burkitt's lymphoma | 0.0 |
| Neuroblastoma | | | |
| (metastasis) | | | |
| SF-295-Glioblastoma | 0.4 | RL-non-Hodgkin's B-cell | 0.0 |
| | | lymphoma | |
| Cerebellum | 15.3 | JM1-pre-B-cell lymphoma | 0.0 |
| Cerebellum | 5.4 | Jurkat-T cell leukemia | 0.2 |
| NCI-H292- | 0.0 | TF-1-Erythroleukemia | 0.0 |
| Mucoepidermoid lung | | | |
| carcinoma | | | |
| DMS-114-Small cell | 0.0 | HUT 78-T-cell lymphoma | 0.0 |
| lung cancer | | | |
| DMS-79-Small cell lung | 3.3 | U937-Histiocytic lymphoma | 0.0 |
| cancer | | | |
| NCI-H146-Small cell | 0.0 | KU-812-Myelogenous | 0.0 |
| lung cancer | | leukemia | |
| NCI-H526-Small cell | 57.4 | 769-P-Clear cell renal | 0.0 |
| lung cancer | | carcinoma | |
| NCI-N417-Small cell | 14.4 | Caki-2-Clear cell renal | 32.8 |
| lung cancer | | carcinoma | |
| NCI-H82-Small cell | 0.0 | SW 839-Clear cell renal | 0.2 |
| lung cancer | | carcinoma | |
| NCI-H157-Squamous | 0.0 | G401-Wilms' tumor | 0.0 |
| cell lung cancer | | | |
| (metastasis) | | | |
| NCI-H1155-Large cell | 2.9 | Hs766T-Pancreatic | 0.0 |
| lung cancer | | carcinoma (LN metastasis) | |
| NCI-H1299-Large cell | 0.3 | CAPAN-1-Pancreatic | 0.0 |
| lung cancer | | adenocarcinoma (liver | |
| | | metastasis) | |
| NCI-H727-Lung | 15.9 | SU86.86-Pancreatic | 1.5 |
| carcinoid | | carcinoma (liver metastasis) | |
| NCI-UMC-11-Lung | 1.2 | BxPC-3-Pancreatic | 0.0 |
| carcinoid | | adenocarcinoma | |
| LX-1-Small cell lung | 0.0 | HPAC-Pancreatic | 0.3 |
| cancer | | adenocarcinoma | |
| Colo-205-Colon cancer | 0.0 | MIA PaCa-2-Pancreatic | 0.0 |
| | | carcinoma | |
| KM12-Colon cancer | 0.0 | CFPAC-1-Pancreatic ductal | 0.0 |
| | | adenocarcinoma | |
| KM20L2-Colon cancer | 0.0 | PANC-1-Pancreatic | 0.8 |
| | | epithelioid ductal carcinoma | |
| NCI-H716-Colon cancer | 100.0 | T24-Bladder carcinma | 0.0 |
| | | (transitional cell) | |
| SW-48-Colon | 0.0 | 5637-Bladder carcinoma | 0.0 |
| adenocarcinoma | | | |
| SW1116-Colon | 0.0 | HT-1197-Bladder carcinoma | 0.0 |
| adenocarcinoma | | | |
| LS 174T-Colon | 0.0 | UM-UC-3-Bladder carcinma | 0.0 |
| adenocarcinoma | | (transitional cell) | |
| SW-948-Colon | 0.0 | A204-Rhabdomyosarcoma | 0.0 |
| adenocarcinoma | | | |
| SW-480-Colon | 1.1 | HT-1080-Fibrosarcoma | 0.3 |
| adenocarcinoma | | | |
| NCI-SNU-5-Gastric | 0.0 | MG-63-Osteosarcoma | 0.0 |
| carcinoma | | | |
| KATO III-Gastric | 0.0 | SK-LMS-1-Leiomyosarcoma | 4.6 |
| carcinoma | | (vulva) | |
| NCI-SNU-16-Gastric | 0.4 | SJRH30-Rhabdomyosarcoma | 0.2 |
| carcinoma | | (met to bone marrow) | |
| NCI-SNU-1-Gastric | 0.0 | A431-Epidermoid carcinoma | 0.0 |
| carcinoma | | | |
| RF-1-Gastric | 0.0 | WM266-4-Melanoma | 0.0 |
| adenocarcinoma | | | |
| RF-48-Gastric | 0.0 | DU 145-Prostate carcinoma | 0.0 |
| adenocarcinoma | | (brain metastasis) | |
| MKN-45-Gastric | 0.0 | MDA-MB-468-Breast | 1.3 |
| carcinoma | | adenocarcinoma | |
| NCI-N87-Gastric | 0.0 | SCC-4-Squamous cell | 0.0 |
| carcinoma | | carcinoma of tongue | |
| OVCAR-5-Ovarian | 0.0 | SCC-9-Squamous cell | 0.0 |
| carcinoma | | carcinoma of tongue | |
| RL95-2-Uterine | 0.2 | SCC-15-Squamous cell | 0.0 |
| carcinoma | | carcinoma of tongue | |
| HelaS3-Cervical | 0.0 | CAL 27-Squamous cell | 0.0 |
| adenocarcinoma | | carcinoma of tongue | |

TABLE AXG
Panel 4D

| | Rel. Exp. (%) | | Rel. Exp. (%) |
|---|---|---|---|
| | Ag3024, Run | | Ag3024, Run |
| Tissue Name | 162427416 | Tissue Name | 162427416 |
| Secondary Th1 act | 0.0 | HUVEC IL-1beta | 0.0 |
| Secondary Th2 act | 0.0 | HUVEC IFN gamma | 31.4 |
| Secondary Tr1 act | 0.0 | HUVEC TNF alpha + IFN | 14.9 |
| | | gamma | |
| Secondary Th1 rest | 0.0 | HUVEC TNF alpha + IL4 | 8.8 |
| Secondary Th2 rest | 4.4 | HUVEC IL-11 | 11.4 |
| Secondary Tr1 rest | 0.0 | Lung Microvascular EC | 10.1 |
| | | none | |
| Primary Th1 act | 0.0 | Lung Microvascular EC | 8.7 |
| | | TNF alpha + IL-1beta | |
| Primary Th2 act | 0.0 | Microvascular Dermal EC | 11.3 |
| | | none | |
| Primary Tr1 act | 0.0 | Microsvasular Dermal EC | 3.0 |
| | | TNF alpha + IL-1beta | |
| Primary Th1 rest | 0.0 | Bronchial epithelium | 5.3 |
| | | TNF alpha + IL1beta | |
| Primary Th2 rest | 0.0 | Small airway epithelium | 3.7 |
| | | none | |
| Primary Tr1 rest | 0.0 | Small airway epithelium | 66.0 |
| | | TNF alpha + IL-1beta | |
| CD45RA CD4 | 0.0 | Coronery artery SMC rest | 21.0 |
| lymphocyte act | | | |
| CD45RO CD4 | 0.0 | Coronery artery SMC | 10.4 |
| lymphocyte act | | TNF alpha + IL-1beta | |
| CD8 lymphocyte act | 0.0 | Astrocytes rest | 0.0 |
| Secondary CD8 | 0.0 | Astrocytes TNF alpha + | 0.0 |
| lymphocyte rest | | IL-1beta | |
| Secondary CD8 | 0.0 | KU-812 (Basophil) rest | 0.0 |
| lymphocyte act | | | |
| CD4 lymphocyte none | 0.0 | KU-812 (Basophil) | 0.0 |
| | | PMA/ionomycin | |
| 2ry Th1/Th2/Tr1_anti- | 0.0 | CCD1106 (Keratinocytes) | 0.0 |
| CD95 CH11 | | none | |
| LAK cells rest | 0.0 | CCD1106 (Keratinocytes) | 0.0 |
| | | TNF alpha + IL-1beta | |
| LAK cells IL-2 | 0.0 | Liver cirrhosis | 17.7 |
| LAK cells IL-2 + IL-12 | 0.0 | Lupus kidney | 5.6 |
| LAK cells IL-2 + IFN | 0.0 | NCI-H292 none | 3.2 |
| gamma | | | |
| LAK cells IL-2 + IL-18 | 0.0 | NCI-H292 IL-4 | 0.0 |
| LAK cells | 0.0 | NCI-H292 IL-9 | 10.4 |
| PMA/ionomycin | | | |
| NK Cells IL-2 rest | 0.0 | NCI-H292 IL-13 | 0.0 |
| Two Way MLR 3 day | 0.0 | NCI-H292 IFN gamma | 0.0 |
| Two Way MLR 5 day | 0.0 | HPAEC none | 6.3 |
| Two Way MLR 7 day | 0.0 | HPAEC TNF alpha + IL-1 | 3.2 |
| | | beta | |
| PBMC rest | 0.0 | Lung fibroblast none | 0.0 |
| PBMC PWM | 0.0 | Lung fibroblast | 0.0 |
| | | TNF alpha + IL-1beta | |
| PBMC PHA-L | 0.0 | Lung fibroblast IL-4 | 0.0 |
| Ramos (B cell) none | 0.0 | Lung fibroblast IL-9 | 0.0 |
| Ramos (B cell) | 4.7 | Lung fibroblast IL-13 | 0.0 |
| ionomycin | | | |
| B lymphocytes PWM | 0.0 | Lung fibroblast IFN | 0.0 |
| | | gamma | |
| B lymphocytes CD40L | 0.0 | Dermal fibroblast | 0.0 |
| and IL-4 | | CCD1070 rest | |
| EOL-1 dbcAMP | 0.0 | Dermal fibroblast | 0.0 |
| | | CCD1070 TNF alpha | |
| EOL-1 dbcAMP | 0.0 | Dermal fibroblast | 4.1 |
| PMA/ionomycin | | CCD1070 IL-1beta | |
| Dendritic cells none | 0.0 | Dermal fibroblast IFN | 6.1 |
| | | gamma | |
| Dendritic cells LPS | 0.0 | Dermal fibroblast IL-4 | 8.7 |
| Dendritic cells anti- | 0.0 | IBD Colitis 2 | 0.0 |
| CD40 | | | |
| Monocytes rest | 0.0 | IBD Crohn's | 7.7 |
| Monocytes LPS | 0.0 | Colon | 24.1 |
| Macrophages rest | 0.0 | Lung | 14.5 |
| Macrophages LPS | 5.3 | Thymus | 100.0 |
| HUVEC none | 24.7 | Kidney | 0.0 |
| HUVEC starved | 11.8 | | |

TABLE AXH
Panel CNS_1

| | Rel. Exp. (%) Ag3024, | | Rel. Exp. (%) Ag3024, |
|---|---|---|---|
| Tissue Name | Run 171694538 | Tissue Name | Run 171694538 |
| BA4 Control | 38.2 | BA17 PSP | 27.7 |
| BA4 Control2 | 73.2 | BA17 PSP2 | 10.7 |
| BA4 | 7.7 | Sub Nigra Control | 11.7 |
| Alzheimer's2 | | | |
| BA4 Parkinson's | 60.7 | Sub Nigra Control2 | 20.4 |
| BA4 | 85.3 | Sub Nigra | 6.4 |
| Parkinson's2 | | Alzheimer's2 | |
| BA4 | 44.1 | Sub Nigra | 29.5 |
| Huntington's | | Parkinson's2 | |
| BA4 | 8.7 | Sub Nigra | 28.9 |
| Huntington's2 | | Huntington's | |
| BA4 PSP | 7.7 | Sub Nigra | 20.7 |
| | | Huntington's2 | |
| BA4 PSP2 | 39.5 | Sub Nigra PSP2 | 1.5 |
| BA4 Depression | 2.1 | Sub Nigra | 0.8 |
| | | Depression | |
| BA4 | 5.8 | Sub Nigra | 4.7 |
| Depression2 | | Depression2 | |
| BA7 Control | 66.0 | Glob Palladus | 3.9 |
| | | Control | |
| BA7 Control2 | 52.5 | Glob Palladus | 6.9 |
| | | Control2 | |
| BA7 | 8.8 | Glob Palladus | 5.5 |
| Alzheimer's2 | | Alzheimer's | |
| BA7 Parkinson's | 20.4 | Glob Palladus | 2.8 |
| | | Alzheimer's2 | |
| BA7 | 64.6 | Glob Palladus | 48.6 |
| Parkinson's2 | | Parkinson's | |
| BA7 | 55.1 | Glob Palladus | 7.1 |
| Huntington's | | Parkinson's2 | |
| BA7 | 54.7 | Glob Palladus PSP | 3.2 |
| Huntington's2 | | | |
| BA7 PSP | 57.8 | Glob Palladus PSP2 | 3.0 |
| BA7 PSP2 | 26.4 | Glob Palladus | 2.1 |
| | | Depression | |
| BA7 Depression | 7.7 | Temp Pole Control | 14.8 |
| BA9 Control | 31.4 | Temp Pole Control2 | 57.0 |
| BA9 Control2 | 100.0 | Temp Pole | 6.1 |
| | | Alzheimer's | |
| BA9 Alzheimer's | 4.8 | Temp Pole | 3.4 |
| | | Alzheimer's2 | |
| BA9 | 14.5 | Temp Pole | 23.2 |
| Alzheimer's2 | | Parkinson's | |
| BA9 Parkinson's | 31.6 | Temp Pole | 30.1 |
| | | Parkinson's2 | |
| BA9 | 56.3 | Temp Pole | 41.8 |
| Parkinson's2 | | Huntington's | |
| BA9 | 53.2 | Temp Pole PSP | 3.5 |
| Huntington's | | | |
| BA9 | 14.3 | Temp Pole PSP2 | 3.3 |
| Huntington's2 | | | |
| BA9 PSP | 17.7 | Temp Pole | 1.9 |
| | | Depression2 | |
| BA9 PSP2 | 5.1 | Cing Gyr Control | 67.8 |
| BA9 Depression | 9.0 | Cing Gyr Control2 | 38.4 |
| BA9 | 7.4 | Cing Gyr | 7.4 |
| Depression2 | | Alzheimer's | |
| BA17 Control | 51.8 | Cing Gyr | 12.2 |
| | | Alzheimer's2 | |
| BA17 Control2 | 54.0 | Cing Gyr Parkinson's | 15.4 |
| BA17 | 2.7 | Cing Gyr | 39.5 |
| Alzheimer's2 | | Parkinson's2 | |
| BA17 | 23.2 | Cing Gyr | 48.0 |
| Parkinson's | | Huntington's | |
| BA17 | 53.6 | Cing Gyr | 14.2 |
| Parkinson's2 | | Huntington's2 | |
| BA17 | 41.2 | Cing Gyr PSP | 13.9 |
| Huntington's | | | |
| BA17 | 9.5 | Cing Gyr PSP2 | 5.0 |
| Huntington's2 | | | |
| BA17 | 6.0 | Cing Gyr Depression | 5.7 |
| Depression | | | |
| BA17 | 14.4 | Cing Gyr | 9.7 |
| Depression2 | | Depression2 | |

AI_comprehensive panel_v1.0 Summary: Ag3024 The NOV55 gene is found at low but significant levels in lung tissue from COPD, emphysema and asthma patients. This expression is consistent with panel 4D which shows expression in small airway epithelium. Therefore, this gene could be a marker or a target for lung inflammatory diseases.

CNS_neurodegeneration_v1.0 Summary: Ag3024 Results of two experiments with the same probe and primer set confirm expression of the NOV55 gene in the brain. Please see Panel 1.3D for discussion of utility of this gene in the central nervous system.

Panel 1.3D Summary: Ag3024 Expression of the NOV55 gene, a heparin sulfate proteoglycan homolog, is highly brain preferential and suggests a role for this gene product in CNS processes. Heparin sulfate proteoglycans (HSPGs) are a component of amyloid plaques in Alzheimer's disease. The interaction of apoE with HSPGs has also been implicated in the pathogenesis of Alzheimer's disease and may play a role in neuronal repair. apoE has an HSPG-binding site highly complementary to heparan sulfates rich in N- and O-sulfo groups. Therefore, enzymes that influence the structure of HSPGs, such as the putative protein product of the NOV55 gene, may influence protein agregation and the functional processes underlying Alzheimer's disease. Thus, agents that target and modulate the activity of this gene product may be effective in the treatment of neurodegenerative diseases including Alzheimer's disease. This gene is also expressed in breast and brain cancer cell lines at low but significant levels. Therefore, the expression of this gene could be of use as a marker for breast and brain cancer. In addition, therapeutic inhibition of the activity of the product of this gene, through the use of antibodies or small molecule drugs, may be useful in the therapy of brain and breast cancer.

References:

Libeu C P, Lund-Katz S, Phillips M C, Wehrli S, Hernaiz M J, Capila I, Linhardt R J, Raffai R L, Newhouse Y M, Zhou F, Weisgraber K H. New insights into the heparan sulfate proteoglycan-binding activity of apolipoprotein E. J Biol Chem Oct. 19, 2001;276(42):39138-44

Defective binding of apolipoprotein E (apoE) to heparan sulfate proteoglycans (HSPGs) is associated with increased risk of atherosclerosis due to inefficient clearance of lipoprotein remnants by the liver. The interaction of apoE with HSPGs has also been implicated in the pathogenesis of Alzheimer's disease and may play a role in neuronal repair. To identify which residues in the heparin-binding site of apoE and which structural elements of heparan sulfate interact, we used a variety of approaches, including glycosaminoglycan specificity assays, (13)C nuclear magnetic resonance, and heparin affinity chromatography. The formation of the high affinity complex required Arg-142, Lys-143, Arg-145, Lys-146, and Arg-147 from apoE and N- and 6-O-sulfo groups of the glucosamine units from the heparin fragment. As shown by molecular modeling, using a high affinity binding octasaccharide fragment of heparin, these findings are consistent with a binding mode in which five saccharide residues of fully sulfated heparan sulfate lie in a shallow groove of the alpha-helix that contains the HSPG-binding site (helix 4 of the four-helix bundle of the 22-kDa fragment). This groove is lined with residues Arg-136, Ser-139, His-140,Arg-142, Lys-143, Arg-145, Lys-146, and Arg-147. In the model, all of these residues make direct contact with either the 2-O-sulfo groups of the iduronic acid monosaccharides or the N- and 6-O-sulfo groups of the glucosamine sulfate monosaccharides. This model indicates that apoE has an HSPG-binding site highly complementary to heparan sulfate rich in N- and O-sulfo groups such as that found in the liver and the brain.

Inoue S. Basement membrane and beta amyloid fibrillogenesis in Alzheimer's disease. Int Rev Cytol 2001;210:121-61

High-resolution ultrastructural and immunohistochemical studies revealed that in situ beta amyloid fibrils of Alzheimer's disease were made up of a core consisting of a solid column of amyloid P component (AP) and associated chondroitin sulfate proteoglycan, and a heparan sulfate proteoglycan surface layer with externally associated fine filaments of beta protein. The main body of beta amyloid fibrils closely resembled that of microfibrils. Abundant microfibrils were reported to be present at the basement membrane of capillaries with “leaky” blood-urine or blood-air barriers. Similarly, abundant microfibril-like beta amyloid fibrils are formed at the microvascular basement membrane in cerebrovascular amyloid angiopathy with altered blood-brain barrier. Since AP is an indispensable major component of microfibrils and microfibril-like structures, the formation of microfibrils may depend on, among other factors, the availability of AP. Thus, in beta amyloid fibrillogenesis fibrils may be built around AP which continuously leaks out from circulation into vascular basement membrane, and beta amyloid fibrils may be regarded as pathologically altered basement membrane-associated microfibrils. With no source of AP around them, senile plaque fibrils may also be derived from perivascular amyloid.

Panel 2D Summary: Ag3024 The NOV55 gene is expressed at low but significant levels in most of the samples on this panel, with highest expression in a kidney cancer sample (CT=30.6). Significant levels of expression are also seen in samples derived from breast and gastric cancer samples.

Therefore, expression of this gene could be of use as a marker for breast and gastric cancer. In addition, therapeutic inhibition of the activity of the product of this gene, through the use of antibodies or small molecule drugs, may be useful in the therapy of breast and gastric cancer.

Panel 3D Summary: Ag3024 The NOV55 gene is expressed at low but significant levels in cell lines from a renal carcinoma, colon cancer, glioblastoma and three lung cancer lines. Thus, this gene could be a marker as well as a target for inhibition in these cancers.

Panel 4D Summary: Ag3024 The NOV55 gene, a heparin Sulfate 6-Sulfotransferase 3 homolog, is expressed at low but significant levels in thymus and small airway epithelium treated with TNFalpha+IL-1 beta (CTs=34). Thus, the NOV55 gene product may be a marker for thymus or activated small airway epithelium.

Panel CNS_{—}1 Summary: Ag3024 Expression of the NOV55 gene in this panel confirms the presence of this gene product in the brain1. Please see Panel 1.3D for discussion of utility of this gene in the central nervous system.

NOV56a and NOV56b

Expression of gene NOV56a and variant NOV56b was assessed using the primer-probe sets Ag3027 and Ag1169, described in Tables AYA and AYB. Results of the RTQ-PCR runs are shown in Tables AYC, AYD, AYE, AYF, AYG, AYH and AYI.

TABLE AYA
Probe Name Ag3027

| | | | Start | |
|---|---|---|---|---|
| Primers | Sequences | Length | Position | SEQ ID NO: |
| Forward | 5′-aaaaccagatttggagttcgtt-3′ | 22 | 355 | 1210 |
| Probe | TET-5′-cttgaaatgtcctcaccacaactgat-3′-TAMRA | 26 | 377 | 1211 |
| Reverse | 5′-tccagatagatggtggaatcag-3′ | 22 | 425 | 1212 |

TABLE AYB
Probe Name Ag1169

| | | | Start | |
|---|---|---|---|---|
| Primers | Sequences | Length | Position | SEQ ID NO: |
| Forward | 5′-aaaaccagatttggagttcgtt-3′ | 22 | 355 | 1213 |
| Probe | TET-5′-cttgaaatgtcctcaccacaactgat-3′-TAMRA | 26 | 377 | 1214 |
| Reverse | 5′-tccagatagatggtggaatcag-3′ | 22 | 425 | 1215 |

TABLE AYC
General_screening_panel_v1.5

| | Rel. Exp. (%) Ag3027, | | Rel. Exp. (%) Ag3027, |
|---|---|---|---|
| Tissue Name | Run 228714682 | Tissue Name | Run 228714682 |
| Adipose | 2.3 | Renal ca. TK-10 | 10.4 |
| Melanoma* | 1.4 | Bladder | 11.0 |
| Hs688(A).T | | | |
| Melanoma* | 1.0 | Gastric ca. (liver met.) | 0.6 |
| Hs688(B).T | | NCI-N87 | |
| Melanoma* M14 | 0.0 | Gastric ca. KATO III | 7.4 |
| Melanoma* | 0.0 | Colon ca. SW-948 | 43.8 |
| LOXIMVI | | | |
| Melanoma* SK- | 1.9 | Colon ca. SW480 | 6.2 |
| MEL-5 | | | |
| Squamous cell | 0.0 | Colon ca.* (SW480 | 9.9 |
| carcinoma SCC-4 | | met) SW620 | |
| Testis Pool | 0.7 | Colon ca. HT29 | 2.0 |
| Prostate ca.* (bone | 0.7 | Colon ca. HCT-116 | 0.5 |
| met) PC-3 | | | |
| Prostate Pool | 3.8 | Colon ca. CaCo-2 | 73.2 |
| Placenta | 0.0 | Colon cancer tissue | 21.8 |
| Uterus Pool | 0.4 | Colon ca. SW1116 | 0.7 |
| Ovarian ca. | 0.0 | Colon ca. Colo-205 | 2.6 |
| OVCAR-3 | | | |
| Ovarian ca. SK-OV-3 | 29.9 | Colon ca. SW-48 | 0.3 |
| Ovarian ca. | 0.8 | Colon Pool | 2.6 |
| OVCAR-4 | | | |
| Ovarian ca. | 11.0 | Small Intestine Pool | 0.8 |
| OVCAR-5 | | | |
| Ovarian ca. IGROV-1 | 6.9 | Stomach Pool | 2.0 |
| Ovarian ca. | 1.1 | Bone Marrow Pool | 0.0 |
| OVCAR-8 | | | |
| Ovary | 0.8 | Fetal Heart | 1.4 |
| Breast ca. MCF-7 | 0.2 | Heart Pool | 0.5 |
| Breast ca. MDA- | 0.5 | Lymph Node Pool | 1.4 |
| MB-231 | | | |
| Breast ca. BT 549 | 0.5 | Fetal Skeletal Muscle | 1.1 |
| Breast ca. T47D | 0.4 | Skeletal Muscle Pool | 0.0 |
| Breast ca. MDA-N | 0.0 | Spleen Pool | 0.0 |
| Breast Pool | 4.9 | Thymus Pool | 0.3 |
| Trachea | 0.3 | CNS cancer (glio/astro) | 4.7 |
| | | U87-MG | |
| Lung | 0.0 | CNS cancer (glio/astro) | 1.6 |
| | | U-118-MG | |
| Fetal Lung | 2.0 | CNS cancer | 0.0 |
| | | (neuro; met) SK-N-AS | |
| Lung ca. NCI-N417 | 0.0 | CNS cancer (astro) SF- | 0.5 |
| | | 539 | |
| Lung ca. LX-1 | 100.0 | CNS cancer (astro) | 4.1 |
| | | SNB-75 | |
| Lung ca. NCI-H146 | 0.3 | CNS cancer (glio) | 13.8 |
| | | SNB-19 | |
| Lung ca. SHP-77 | 0.0 | CNS cancer (glio) SF- | 3.6 |
| | | 295 | |
| Lung ca. A549 | 1.4 | Brain (Amygdala) Pool | 0.2 |
| Lung ca. NCI-H526 | 1.0 | Brain (cerebellum) | 0.1 |
| Lung ca. NCI-H23 | 2.6 | Brain (fetal) | 0.2 |
| Lung ca. NCI-H460 | 1.2 | Brain (Hippocampus) | 0.3 |
| | | Pool | |
| Lung ca. HOP-62 | 0.2 | Cerebral Cortex Pool | 0.5 |
| Lung ca. NCI-H522 | 0.7 | Brain (Substantia nigra) | 0.0 |
| | | Pool | |
| Liver | 0.0 | Brain (Thalamus) Pool | 0.4 |
| Fetal Liver | 3.2 | Brain (whole) | 0.6 |
| Liver ca. HepG2 | 0.5 | Spinal Cord Pool | 0.3 |
| Kidney Pool | 1.8 | Adrenal Gland | 1.1 |
| Fetal Kidney | 29.9 | Pituitary gland Pool | 0.3 |
| Renal ca. 786-0 | 1.8 | Salivary Gland | 0.2 |
| Renal ca. A498 | 0.7 | Thyroid (female) | 0.1 |
| Renal ca. ACHN | 3.2 | Pancreatic ca. CAPAN2 | 3.1 |
| Renal ca. UO-31 | 3.2 | Pancreas Pool | 4.4 |

TABLE AYD
Panel 1.2

| | Rel. Exp. (%) | Rel. Exp. (%) | | Rel. Exp. (%) | Rel. Exp. (%) |
|---|---|---|---|---|---|
| | Ag1169, Run | Ag1169, Run | | Ag1169, Run | Ag1169, Run |
| Tissue Name | 129128191 | 129656838 | Tissue Name | 129128191 | 129656838 |
| Endothelial cells | 0.0 | 24.3 | Renal ca. 786-0 | 0.2 | 10.5 |
| Heart (Fetal) | 0.0 | 57.0 | Renal ca. A498 | 5.5 | 10.9 |
| Pancreas | 0.0 | 0.0 | Renal ca. RXF | 0.9 | 14.9 |
| | | | 393 | | |
| Pancreatic ca. | 0.2 | 0.0 | Renal ca. | 1.5 | 22.8 |
| CAPAN 2 | | | ACHN | | |
| Adrenal Gland | 0.0 | 7.0 | Renal ca. UO- | 5.6 | 12.3 |
| | | | 31 | | |
| Thyroid | 0.1 | 0.1 | Renal ca. TK- | 11.3 | 1.7 |
| | | | 10 | | |
| Salivary gland | 0.1 | 6.2 | Liver | 0.0 | 12.9 |
| Pituitary gland | 0.0 | 0.3 | Liver (fetal) | 0.0 | 1.4 |
| Brain (fetal) | 0.0 | 0.0 | Liver ca. | 0.2 | 4.5 |
| | | | (hepatoblast) | | |
| | | | HepG2 | | |
| Brain (whole) | 0.0 | 4.5 | Lung | 0.0 | 14.9 |
| Brain | 0.0 | 6.3 | Lung (fetal) | 0.0 | 22.2 |
| (amygdala) | | | | | |
| Brain | 0.0 | 0.3 | Lung ca. | 27.0 | 74.2 |
| (cerebellum) | | | (small cell) | | |
| | | | LX-1 | | |
| Brain | 0.0 | 14.4 | Lung ca. | 0.0 | 0.0 |
| (hippocampus) | | | (small cell) | | |
| | | | NCI-H69 | | |
| Brain (thalamus) | 0.0 | 1.2 | Lung ca. (s.cell | 0.0 | 0.0 |
| | | | var.) SHP-77 | | |
| Cerebral Cortex | 0.0 | 15.5 | Lung ca. (large | 0.0 | 0.0 |
| | | | cell)NCI-H460 | | |
| Spinal cord | 0.0 | 22.4 | Lung ca. (non- | 0.9 | 0.0 |
| | | | sm. cell) A549 | | |
| glio/astro U87- | 0.2 | 0.0 | Lung ca. (non- | 0.3 | 0.0 |
| MG | | | s.cell) NCI- | | |
| | | | H23 | | |
| glio/astro U- | 0.0 | 0.0 | Lung ca. (non- | 0.1 | 0.6 |
| 118-MG | | | s.cell) HOP-62 | | |
| astrocytoma | 0.0 | 0.0 | Lung ca. (non- | 0.1 | 0.1 |
| SW1783 | | | s.cl) NCI-H522 | | |
| neuro*; met SK- | 0.0 | 0.0 | Lung ca. | 6.3 | 0.0 |
| N-AS | | | (squam.) SW | | |
| | | | 900 | | |
| astrocytoma SF- | 0.0 | 0.0 | Lung ca. | 0.0 | 0.0 |
| 539 | | | (squam.) NCI- | | |
| | | | H596 | | |
| astrocytoma | 0.0 | 0.0 | Mammary | 100.0 | 100.0 |
| SNB-75 | | | gland | | |
| glioma SNB-19 | 0.4 | 0.6 | Breast ca.* | 0.0 | 4.0 |
| | | | (pl.ef) MCF-7 | | |
| glioma U251 | 0.1 | 1.7 | Breast ca.* | 0.0 | 0.0 |
| | | | (pl.ef) MDA- | | |
| | | | MB-231 | | |
| glioma SF-295 | 0.5 | 0.0 | Breast ca.* (pl. | 0.0 | 1.4 |
| | | | ef) T47D | | |
| Heart | 0.0 | 58.2 | Breast ca. BT- | 0.0 | 0.1 |
| | | | 549 | | |
| Skeletal Muscle | 0.0 | 9.3 | Breast ca. | 0.0 | 0.0 |
| | | | MDA-N | | |
| Bone marrow | 0.0 | 0.0 | Ovary | 0.0 | 14.0 |
| Thymus | 0.0 | 0.0 | Ovarian ca. | 0.0 | 5.2 |
| | | | OVCAR-3 | | |
| Spleen | 0.0 | 0.0 | Ovarian ca. | 0.0 | 15.3 |
| | | | OVCAR-4 | | |
| Lymph node | 0.0 | 3.0 | Ovarian ca. | 50.7 | 15.7 |
| | | | OVCAR-5 | | |
| Colorectal | 0.1 | 0.0 | Ovarian ca. | 1.5 | 0.1 |
| Tissue | | | OVCAR-8 | | |
| Stomach | 8.4 | 23.0 | Ovarian ca. | 43.5 | 17.0 |
| | | | IGROV-1 | | |
| Small intestine | 0.2 | 10.0 | Ovarian ca. | 13.5 | 17.0 |
| | | | (ascites) SK- | | |
| | | | OV-3 | | |
| Colon ca. | 0.6 | 0.0 | Uterus | 0.2 | 7.7 |
| SW480 | | | | | |
| Colon ca.* | 7.5 | 0.4 | Placenta | 0.0 | 75.8 |
| SW620 (SW480 | | | | | |
| met) | | | | | |
| Colon ca. HT29 | 0.0 | 0.0 | Prostate | 12.1 | 13.7 |
| Colon ca. HCT- | 0.0 | 0.0 | Prostate ca.* | 0.0 | 10.4 |
| 116 | | | (bone met) PC-3 | | |
| Colon ca. CaCo-2 | 34.4 | 11.8 | Testis | 0.6 | 0.2 |
| Colon ca. Tissue | 11.4 | 15.9 | Melanoma | 0.0 | 0.0 |
| (ODO3866) | | | Hs688(A).T | | |
| Colon ca. HCC- | 2.0 | 0.0 | Melanoma* | 0.2 | 0.0 |
| 2998 | | | (met) | | |
| | | | Hs688(B).T | | |
| Gastric ca.* | 0.0 | 0.0 | Melanoma | 0.0 | 0.0 |
| (liver met) NCI- | | | UACC-62 | | |
| N87 | | | | | |
| Bladder | 5.3 | 11.3 | Melanoma | 0.0 | 0.0 |
| | | | M14 | | |
| Trachea | 0.0 | 2.6 | Melanoma | 0.0 | 0.0 |
| | | | LOX IMVI | | |
| Kidney | 4.5 | 14.0 | Melanoma* | 0.0 | 0.0 |
| | | | (met) SK- | | |
| | | | MEL-5 | | |
| Kidney (fetal) | 14.3 | 31.2 | | | |

TABLE AYE
Panel 1.3D

| | Rel. Exp. (%) | Rel. Exp. (%) | | Rel. Exp. (%) | Rel. Exp. (%) |
|---|---|---|---|---|---|
| | Ag1169, Run | Ag3027, Run | | Ag1169, Run | Ag3027, Run |
| Tissue Name | 165518394 | 165519993 | Tissue Name | 165518394 | 165519993 |
| Liver | 0.5 | 0.4 | Kidney (fetal) | 0.7 | 0.0 |
| adenocarcinoma | | | | | |
| Pancreas | 0.0 | 0.0 | Renal ca. 786-0 | 0.5 | 0.6 |
| Pancreatic ca. | 0.7 | 0.4 | Renal ca. | 0.9 | 1.6 |
| CAPAN 2 | | | A498 | | |
| Adrenal gland | 0.0 | 0.0 | Renal ca. RXF | 0.0 | 0.9 |
| | | | 393 | | |
| Thyroid | 0.0 | 0.0 | Renal ca. | 0.0 | 0.0 |
| | | | ACHN | | |
| Salivary gland | 0.0 | 0.0 | Renal ca. UO- | 0.0 | 0.0 |
| | | | 31 | | |
| Pituitary gland | 0.0 | 0.0 | Renal ca. TK- | 2.1 | 0.4 |
| | | | 10 | | |
| Brain (fetal) | 0.0 | 0.0 | Liver | 0.0 | 0.0 |
| Brain (whole) | 0.0 | 0.0 | Liver (fetal) | 0.0 | 0.0 |
| Brain (amygdala) | 0.0 | 0.0 | Liver ca. | 0.0 | 0.0 |
| | | | (hepatoblast) | | |
| | | | HepG2 | | |
| Brain (cerebellum) | 0.0 | 0.0 | Lung | 0.0 | 0.0 |
| Brain | 0.0 | 0.4 | Lung (fetal) | 0.0 | 0.0 |
| (hippocampus) | | | | | |
| Brain (substantia | 0.3 | 0.0 | Lung ca. | 21.5 | 12.0 |
| nigra) | | | (small cell) | | |
| | | | LX-1 | | |
| Brain (thalamus) | 0.0 | 0.0 | Lung ca. | 0.0 | 0.0 |
| | | | (small cell) | | |
| | | | NCI-H69 | | |
| Cerebral Cortex | 0.0 | 0.5 | Lung ca. | 0.0 | 0.0 |
| | | | (s.cell var.) | | |
| | | | SHP-77 | | |
| Spinal cord | 0.0 | 0.3 | Lung ca. (large | 0.0 | 1.3 |
| | | | cell) NCI-H460 | | |
| glio/astro U87-MG | 0.0 | 0.3 | Lung ca. (non- | 0.0 | 0.0 |
| | | | sm. cell) A549 | | |
| glio/astro U-118- | 0.0 | 0.3 | Lung ca. (non- | 0.7 | 0.3 |
| MG | | | s.cell) NCI- | | |
| | | | H23 | | |
| astrocytoma | 0.0 | 0.0 | Lung ca. (non- | 0.0 | 0.0 |
| SW1783 | | | s.cell) HOP-62 | | |
| neuro*; met SK-N- | 0.0 | 0.0 | Lung ca. (non- | 0.0 | 0.0 |
| AS | | | s.cl) NCI- | | |
| | | | H522 | | |
| astrocytoma SF- | 0.3 | 0.0 | Lung ca. | 2.8 | 0.0 |
| 539 | | | (squam.) SW | | |
| | | | 900 | | |
| astrocytoma SNB- | 1.9 | 0.9 | Lung ca. | 0.0 | 0.0 |
| 75 | | | (squam.) NCI- | | |
| | | | H596 | | |
| glioma SNB-19 | 0.0 | 0.4 | Mammary | 100.0 | 100.0 |
| | | | gland | | |
| glioma U251 | 0.4 | 0.4 | Breast ca.* | 0.5 | 0.0 |
| | | | (pl.ef) MCF-7 | | |
| glioma SF-295 | 0.0 | 0.0 | Breast ca.* | 0.0 | 0.3 |
| | | | (pl.ef) MDA- | | |
| | | | MB-231 | | |
| Heart (fetal) | 0.0 | 0.0 | Breast ca.* | 0.0 | 0.0 |
| | | | (pl.ef) T47D | | |
| Heart | 0.0 | 0.0 | Breast ca. BT- | 0.3 | 0.0 |
| | | | 549 | | |
| Skeletal muscle | 0.0 | 0.0 | Breast ca. | 0.0 | 0.0 |
| (fetal) | | | MDA-N | | |
| Skeletal muscle | 0.0 | 0.0 | Ovary | 0.0 | 0.0 |
| Bone marrow | 0.0 | 0.0 | Ovarian ca. | 0.0 | 0.0 |
| | | | OVCAR-3 | | |
| Thymus | 0.9 | 0.0 | Ovarian ca. | 0.4 | 0.0 |
| | | | OVCAR-4 | | |
| Spleen | 0.7 | 0.0 | Ovarian ca. | 0.9 | 1.4 |
| | | | OVCAR-5 | | |
| Lymph node | 0.0 | 0.0 | Ovarian ca. | 0.0 | 0.0 |
| | | | OVCAR-8 | | |
| Colorectal | 0.9 | 0.2 | Ovarian ca. | 1.0 | 2.3 |
| | | | IGROV-1 | | |
| Stomach | 1.0 | 0.6 | Ovarian ca.* | 1.4 | 1.8 |
| | | | (ascites) SK- | | |
| | | | OV-3 | | |
| Small intestine | 0.7 | 0.0 | Uterus | 0.5 | 0.0 |
| Colon ca. SW480 | 0.8 | 0.4 | Placenta | 0.0 | 0.0 |
| Colon ca.* | 0.6 | 0.4 | Prostate | 0.3 | 0.5 |
| SW620 (SW480 | | | | | |
| met) | | | | | |
| Colon ca. HT29 | 0.0 | 0.8 | Prostate ca.* | 0.0 | 0.0 |
| | | | (bone met) PC-3 | | |
| Colon ca. HCT- | 0.9 | 0.3 | Testis | 0.8 | 1.2 |
| 116 | | | | | |
| Colon ca. CaCo-2 | 6.7 | 8.1 | Melanoma | 0.0 | 0.0 |
| | | | Hs688(A).T | | |
| Colon ca. | 2.9 | 2.0 | Melanoma* | 0.0 | 0.0 |
| tissue (ODO3866) | | | (met) | | |
| | | | Hs688 (B).T | | |
| Colon ca. HCC- | 0.4 | 0.3 | Melanoma | 0.0 | 0.0 |
| 2998 | | | UACC-62 | | |
| Gastric ca.* (liver | 0.3 | 0.0 | Melanoma | 0.0 | 0.0 |
| met) NCI-N87 | | | M14 | | |
| Bladder | 1.7 | 0.4 | Melanoma | 0.0 | 0.0 |
| | | | LOX IMVI | | |
| Trachea | 0.0 | 0.0 | Melanoma* | 0.0 | 0.0 |
| | | | (met) SK- | | |
| | | | MEL-5 | | |
| Kidney | 2.7 | 1.3 | Adipose | 0.7 | 0.0 |

TABLE AYF
Panel 2D

| | Rel. Exp. (%) | | Rel. Exp. (%) |
|---|---|---|---|
| | Ag3027, Run | | Ag3027, Run |
| Tissue Name | 163577594 | Tissue Name | 163577594 |
| Normal Colon | 14.6 | Kidney Margin | 5.9 |
| | | 8120608 | |
| CC Well to Mod Diff | 15.1 | Kidney Cancer | 34.4 |
| (ODO3866) | | 8120613 | |
| CC Margin (ODO3866) | 0.0 | Kidney Margin | 5.7 |
| | | 8120614 | |
| CC Gr.2 rectosigmoid | 15.5 | Kidney Cancer | 10.5 |
| (ODO3868) | | 9010320 | |
| CC Margin (ODO3868) | 0.7 | Kidney Margin | 25.0 |
| | | 9010321 | |
| CC Mod Diff (ODO3920) | 64.2 | Normal Uterus | 0.0 |
| CC Margin (ODO3920) | 2.2 | Uterus Cancer 064011 | 18.4 |
| CC Gr.2 ascend colon | 11.2 | Normal Thyroid | 1.6 |
| (ODO3921) | | | |
| CC Margin (ODO3921) | 1.8 | Thyroid Cancer | 1.6 |
| | | 064010 | |
| CC from Partial | 16.0 | Thyroid Cancer | 0.0 |
| Hepatectomy (ODO4309) | | A302152 | |
| Mets | | | |
| Liver Margin (ODO4309) | 0.3 | Thyroid Margin | 0.0 |
| | | A302153 | |
| Colon mets to lung | 6.8 | Normal Breast | 28.7 |
| (OD04451-01) | | | |
| Lung Margin (OD04451- | 0.0 | Breast Cancer | 0.5 |
| 02) | | (OD04566) | |
| Normal Prostate 6546-1 | 14.9 | Breast Cancer | 0.0 |
| | | (OD04590-01) | |
| Prostate Cancer | 29.7 | Breast Cancer Mets | 3.7 |
| (OD04410) | | (OD04590-03) | |
| Prostate Margin | 19.1 | Breast Cancer | 1.7 |
| (OD04410) | | Metastasis | |
| | | (OD04655-05) | |
| Prostate Cancer | 4.5 | Breast Cancer 064006 | 0.4 |
| (OD04720-01) | | | |
| Prostate Margin | 3.0 | Breast Cancer 1024 | 55.1 |
| (OD04720-02) | | | |
| Normal Lung 061010 | 0.5 | Breast Cancer | 1.9 |
| | | 9100266 | |
| Lung Met to Muscle | 0.0 | Breast Margin | 8.2 |
| (ODO4286) | | 9100265 | |
| Muscle Margin | 0.9 | Breast Cancer | 18.3 |
| (ODO4286) | | A209073 | |
| Lung Malignant Cancer | 11.0 | Breast Margin | 34.6 |
| (OD03126) | | A2090734 | |
| Lung Margin (OD03126) | 1.1 | Normal Liver | 0.0 |
| Lung Cancer (OD04404) | 1.8 | Liver Cancer 064003 | 15.0 |
| Lung Margin (OD04404) | 0.4 | Liver Cancer 1025 | 0.0 |
| Lung Cancer (OD04565) | 0.5 | Liver Cancer 1026 | 0.0 |
| Lung Margin (OD04565) | 2.1 | Liver Cancer 6004-T | 0.0 |
| Lung Cancer (OD04237- | 3.3 | Liver Tissue 6004-N | 1.1 |
| 01) | | | |
| Lung Margin (OD04237- | 0.0 | Liver Cancer 6005-T | 2.6 |
| 02) | | | |
| Ocular Mel Met to Liver | 1.6 | Liver Tissue 6005-N | 0.0 |
| (ODO4310) | | | |
| Liver Margin (ODO4310) | 0.0 | Normal Bladder | 13.5 |
| Melanoma Mets to Lung | 0.0 | Bladder Cancer 1023 | 32.5 |
| (OD04321) | | | |
| Lung Margin (OD04321) | 0.0 | Bladder Cancer | 2.6 |
| | | A302173 | |
| Normal Kidney | 14.6 | Bladder Cancer | 0.0 |
| | | (OD04718-01) | |
| Kidney Ca, Nuclear grade | 3.3 | Bladder Normal | 1.0 |
| 2 (OD04338) | | Adjacent (OD04718- | |
| | | 03) | |
| Kidney Margin | 5.1 | Normal Ovary | 0.0 |
| (OD04338) | | | |
| Kidney Ca Nuclear grade | 7.9 | Ovarian Cancer | 1.8 |
| 1/2 (OD04339) | | 064008 | |
| Kidney Margin | 27.0 | Ovarian Cancer | 0.0 |
| (OD04339) | | (OD04768-07) | |
| Kidney Ca, Clear cell | 3.4 | Ovary Margin | 0.5 |
| type (OD04340) | | (OD04768-08) | |
| Kidney Margin | 61.6 | Normal Stomach | 45.7 |
| (OD04340) | | | |
| Kidney Ca, Nuclear grade | 0.4 | Gastric Cancer | 0.0 |
| 3 (OD04348) | | 9060358 | |
| Kidney Margin | 84.1 | Stomach Margin | 8.2 |
| (OD04348) | | 9060359 | |
| Kidney Cancer | 3.5 | Gastric Cancer | 6.4 |
| (OD04622-01) | | 9060395 | |
| Kidney Margin | 1.4 | Stomach Margin | 12.9 |
| (OD04622-03) | | 9060394 | |
| Kidney Cancer | 39.2 | Gastric Cancer | 42.9 |
| (OD04450-01) | | 9060397 | |
| Kidney Margin | 51.8 | Stomach Margin 9060396 | 31.4 |
| (OD04450-03) | | | |
| Kidney Cancer 8120607 | 4.0 | Gastric Cancer | 100.0 |
| | | 064005 | |

TABLE AYG
Panel 3D

| | Rel. Exp. (%) | | Rel. Exp. (%) |
|---|---|---|---|
| | Ag1169, Run | | Ag1169, Run |
| Tissue Name | 164038616 | Tissue Name | 164038616 |
| Daoy-Medulloblastoma | 1.9 | Ca Ski-Cervical epidermoid | 0.0 |
| | | carcinoma (metastasis) | |
| TE671-Medulloblastoma | 0.0 | ES-2-Ovarian clear cell | 0.0 |
| | | carcinoma | |
| D283 Med- | 0.0 | Ramos-Stimulated with | 0.0 |
| Medulloblastoma | | PMA/ionomycin 6 h | |
| PFSK-1-Primitive | 0.0 | Ramos-Stimulated with | 0.0 |
| Neuroectodermal | | PMA/ionomycin 14 h | |
| XF-498-CNS | 0.0 | MEG-01-Chronic | 0.0 |
| | | myelogenous leukemia | |
| | | (megokaryoblast) | |
| SNB-78-Glioma | 0.0 | Raji-Burkitt's lymphoma | 0.0 |
| SF-268-Glioblastoma | 0.0 | Daudi-Burkitt's lymphoma | 0.0 |
| T98G-Glioblastoma | 0.0 | U266-B-cell plasmacytoma | 0.0 |
| SK-N-SH- | 0.0 | CA46-Burkitt's lymphoma | 0.0 |
| Neuroblastoma | | | |
| (metastasis) | | | |
| SF-295-Glioblastoma | 0.0 | RL-non-Hodgkin's B-cell | 0.0 |
| | | lymphoma | |
| Cerebellum | 0.0 | JM1-pre-B-cell lymphoma | 0.0 |
| Cerebellum | 0.0 | Jurkat-T cell leukemia | 0.0 |
| NCI-H292- | 1.8 | TF-1-Erythroleukemia | 0.0 |
| Mucoepidermoid lung | | | |
| carcinoma | | | |
| DMS-114-Small cell | 0.0 | HUT 78-T-cell lymphoma | 0.0 |
| lung cancer | | | |
| DMS-79-Small cell lung | 100.0 | U937-Histiocytic lymphoma | 0.0 |
| cancer | | | |
| NCI-H146-Small cell | 0.0 | KU-812-Myelogenous | 0.0 |
| lung cancer | | leukemia | |
| NCI-H526-Small cell | 2.6 | 769-P-Clear cell renal | 1.0 |
| lung cancer | | carcinoma | |
| NCI-N417-Small cell | 0.0 | Caki-2-Clear cell renal | 2.3 |
| lung cancer | | carcinoma | |
| NCI-H82-Small cell | 0.0 | SW 839-Clear cell renal | 3.7 |
| lung cancer | | carcinoma | |
| NCI-H157-Squamous | 0.0 | G401-Wilms' tumor | 0.0 |
| cell lung cancer | | | |
| (metastasis) | | | |
| NCI-H1155-Large cell | 0.0 | Hs766T-Pancreatic | 0.0 |
| lung cancer | | carcinoma (LN metastasis) | |
| NCI-H1299-Large cell | 0.0 | CAPAN-1-Pancreatic | 3.5 |
| lung cancer | | adenocarcinoma (liver | |
| | | metastasis) | |
| NCI-H727-Lung | 0.0 | SU86.86-Pancreatic | 2.3 |
| carcinoid | | carcinoma (liver metastasis) | |
| NCI-UMC-11-Lung | 1.7 | BxPC-3-Pancreatic | 1.2 |
| carcinoid | | adenocarcinoma | |
| LX-1-Small cell lung | 20.6 | HPAC-Pancreatic | 3.3 |
| cancer | | adenocarcinoma | |
| Colo-205-Colon cancer | 0.0 | MIA PaCa-2-Pancreatic | 0.0 |
| | | carcinoma | |
| KM12-Colon cancer | 12.3 | CFPAC-1-Pancreatic ductal | 2.6 |
| | | adenocarcinoma | |
| KM20L2-Colon cancer | 0.0 | PANC-1-Pancreatic | 0.0 |
| | | epithelioid ductal carcinoma | |
| NCI-H716-Colon cancer | 0.0 | T24-Bladder carcinma | 0.0 |
| | | (transitional cell) | |
| SW-48-Colon | 6.0 | 5637-Bladder carcinoma | 0.0 |
| adenocarcinoma | | | |
| SW1116-Colon | 2.6 | HT-1197-Bladder carcinoma | 0.0 |
| adenocarcinoma | | | |
| LS 174T-Colon | 25.5 | UM-UC-3-Bladder carcinma | 0.0 |
| adenocarcinoma | | (transitional cell) | |
| SW-948-Colon | 10.7 | A204-Rhabdomyosarcoma | 0.0 |
| adenocarcinoma | | | |
| SW-480-Colon | 0.0 | HT-1080-Fibrosarcoma | 1.0 |
| adenocarcinoma | | | |
| NCI-SNU-5-Gastric | 1.0 | MG-63-Osteosarcoma | 0.0 |
| carcinoma | | | |
| KATO III-Gastric | 0.0 | SK-LMS-1-Leiomyosarcoma | 0.0 |
| carcinoma | | (vulva) | |
| NCI-SNU-16-Gastric | 0.0 | SJRH30-Rhabdomyosarcoma | 2.0 |
| carcinoma | | (met to bone marrow) | |
| NCI-SNU-1-Gastric | 29.7 | A431-Epidermoid carcinoma | 0.0 |
| carcinoma | | | |
| RF-1-Gastric | 0.0 | WM266-4-Melanoma | 0.0 |
| adenocarcinoma | | | |
| RF-48-Gastric | 0.0 | DU 145-Prostate carcinoma | 0.0 |
| adenocarcinoma | | (brain metastasis) | |
| MKN-45-Gastric | 3.5 | MDA-MB-468-Breast | 0.0 |
| carcinoma | | adenocarcinoma | |
| NCI-N87-Gastric | 0.0 | SCC-4-Squamous cell | 0.0 |
| carcinoma | | carcinoma of tongue | |
| OVCAR-5-Ovarian | 0.0 | SCC-9-Squamous cell | 0.0 |
| carcinoma | | carcinoma of tongue | |
| RL95-2-Uterine | 0.0 | SCC-15-Squamous cell | 0.0 |
| carcinoma | | carcinoma of tongue | |
| HelaS3-Cervical | 0.0 | CAL 27-Squamous cell | 0.0 |
| adenocarcinoma | | carcinoma of tongue | |

TABLE AYH
Panel 4D

| | Rel. | Rel. | Rel. | | Rel. | Rel. | Rel. |
|---|---|---|---|---|---|---|---|
| | Exp. (%) | Exp. (%) | Exp. (%) | | Exp. (%) | Exp. (%) | Exp. (%) |
| | Ag1169, | Ag1169, | Ag3027, | | Ag1169, | Ag1169, | Ag3027, |
| Tissue | Run | Run | Run | Tissue | Run | Run | Run |
| Name | 139591349 | 145735616 | 162426723 | Name | 139591349 | 145735616 | 162426723 |
| Secondary | 0.0 | 0.0 | 0.0 | HUVEC | 0.0 | 0.0 | 0.0 |
| Th1 act | | | | IL-1beta | | | |
| Secondary | 0.0 | 0.0 | 0.0 | HUVEC | 0.0 | 0.0 | 0.7 |
| Th2 act | | | | IFN | | | |
| | | | | gamma | | | |
| Secondary | 0.0 | 0.0 | 0.0 | HUVEC | 0.0 | 0.0 | 0.0 |
| Tr1 act | | | | TNF | | | |
| | | | | alpha + IFN | | | |
| | | | | gamma | | | |
| Secondary | 0.0 | 0.0 | 0.0 | HUVEC | 0.4 | 0.0 | 0.0 |
| Th1 rest | | | | TNF | | | |
| | | | | alpha + IL4 | | | |
| Secondary | 0.0 | 0.0 | 0.0 | HUVEC | 0.0 | 0.0 | 0.0 |
| Th2 rest | | | | IL-11 | | | |
| Secondary | 0.0 | 0.0 | 0.0 | Lung | 0.4 | 0.0 | 0.0 |
| Tr1 rest | | | | Microvascular | | | |
| | | | | EC | | | |
| | | | | none | | | |
| Primary | 0.0 | 0.0 | 0.0 | Lung | 0.9 | 0.0 | 0.0 |
| Th1 act | | | | Microvascular | | | |
| | | | | EC | | | |
| | | | | TNF | | | |
| | | | | alpha + IL-1beta | | | |
| Primary | 0.0 | 0.0 | 0.0 | Microvascular | 0.5 | 0.0 | 3.1 |
| Th2 act | | | | Dermal EC | | | |
| | | | | none | | | |
| Primary | 0.0 | 0.0 | 0.0 | Microsvasular | 0.0 | 0.0 | 0.0 |
| Tr1 act | | | | Dermal EC | | | |
| | | | | TNF | | | |
| | | | | alpha + IL-1beta | | | |
| Primary | 0.0 | 0.0 | 0.0 | Bronchial | 1.0 | 0.0 | 5.1 |
| Th1 rest | | | | epithelium | | | |
| | | | | TNF | | | |
| | | | | alpha + IL-1beta | | | |
| Primary | 0.0 | 0.0 | 0.0 | Small | 0.0 | 0.0 | 2.0 |
| Th2 rest | | | | airway | | | |
| | | | | epithelium | | | |
| | | | | none | | | |
| Primary | 0.0 | 0.0 | 0.0 | Small | 0.0 | 1.3 | 1.9 |
| Tr1 rest | | | | airway | | | |
| | | | | epithelium | | | |
| | | | | TNF | | | |
| | | | | alpha + IL-1beta | | | |
| CD45RA | 0.0 | 0.0 | 0.0 | Coronery | 0.0 | 0.0 | 0.0 |
| CD4 | | | | artery | | | |
| lymphocyte | | | | SMC rest | | | |
| act | | | | | | | |
| CD45RO | 0.0 | 0.0 | 0.0 | Coronery | 0.0 | 0.0 | 0.0 |
| CD4 | | | | artery | | | |
| lymphocyte | | | | SMC | | | |
| act | | | | TNF | | | |
| | | | | alpha + IL-1beta | | | |
| CD8 | 0.0 | 0.0 | 0.0 | Astrocytes | 0.0 | 0.0 | 0.0 |
| lymphocyte | | | | rest | | | |
| act | | | | | | | |
| Secondary | 0.0 | 0.0 | 0.0 | Astrocytes | 1.4 | 3.1 | 0.6 |
| CD8 | | | | TNF | | | |
| lymphocyte | | | | alpha + IL-1beta | | | |
| rest | | | | | | | |
| Secondary | 0.0 | 0.0 | 0.0 | KU-812 | 0.0 | 0.0 | 0.0 |
| CD8 | | | | (Basophil) | | | |
| lymphocyte | | | | rest | | | |
| act | | | | | | | |
| CD4 | 0.0 | 0.0 | 0.0 | KU-812 | 0.0 | 0.0 | 0.0 |
| lymphocyte | | | | (Basophil) | | | |
| none | | | | PMA/ionomycin | | | |
| 2ry | 0.0 | 0.0 | 0.0 | CCD1106 | 0.0 | 0.0 | 0.0 |
| Th1/Th2/Tr1_{—} | | | | (Keratinocytes) | | | |
| anti- | | | | none | | | |
| CD95 | | | | | | | |
| CH11 | | | | | | | |
| LAK cells | 0.0 | 0.0 | 0.0 | CCD1106 | 0.0 | 0.0 | 0.0 |
| rest | | | | (Keratinocytes) | | | |
| | | | | TNF | | | |
| | | | | alpha + IL-1beta | | | |
| LAK cells | 0.0 | 0.0 | 0.0 | Liver | 2.1 | 1.3 | 3.2 |
| IL-2 | | | | cirrhosis | | | |
| LAK cells | 0.0 | 0.0 | 0.0 | Lupus | 5.3 | 3.7 | 4.8 |
| IL-2 + IL- | | | | kidney | | | |
| 12 | | | | | | | |
| LAK cells | 0.0 | 0.0 | 0.0 | NCI-H292 | 0.9 | 0.0 | 2.2 |
| IL-2 + IFN | | | | none | | | |
| gamma | | | | | | | |
| LAK cells | 0.0 | 0.0 | 0.0 | NCI-H292 | 0.0 | 1.2 | 0.7 |
| IL-2 + IL- | | | | IL-4 | | | |
| 18 | | | | | | | |
| LAK cells | 0.0 | 0.0 | 0.0 | NCI-H292 | 0.3 | 0.0 | 0.9 |
| PMA/ionomycin | | | | IL-9 | | | |
| NK Cells | 0.0 | 0.0 | 0.0 | NCI-H292 | 0.0 | 0.0 | 0.7 |
| IL-2 rest | | | | IL-13 | | | |
| Two Way | 0.0 | 0.0 | 0.0 | NCI-H292 | 0.0 | 3.7 | 0.0 |
| MLR 3 | | | | IFN | | | |
| day | | | | gamma | | | |
| Two Way | 0.0 | 0.0 | 0.0 | HPAEC | 36.1 | 0.0 | 1.1 |
| MLR 5 | | | | none | | | |
| day | | | | | | | |
| Two Way | 0.0 | 0.0 | 1.1 | HPAEC | 0.0 | 0.0 | 0.0 |
| MLR 7 | | | | TNF | | | |
| day | | | | alpha + IL-1beta | | | |
| PBMC rest | 0.0 | 0.0 | 0.0 | Lung | 0.0 | 0.0 | 0.0 |
| | | | | fibroblast | | | |
| | | | | none | | | |
| PBMC | 0.0 | 1.4 | 0.0 | Lung | 0.0 | 0.0 | 0.0 |
| PWM | | | | fibroblast | | | |
| | | | | TNF | | | |
| | | | | alpha + IL-1beta | | | |
| PBMC | 0.5 | 0.0 | 1.0 | Lung | 0.0 | 0.0 | 0.0 |
| PHA-L | | | | fibroblast | | | |
| | | | | IL-4 | | | |
| Ramos (B | 0.0 | 1.8 | 0.0 | Lung | 0.0 | 0.0 | 2.0 |
| cell) none | | | | fibroblast | | | |
| | | | | IL-9 | | | |
| Ramos (B | 0.5 | 0.0 | 0.0 | Lung | 0.0 | 0.0 | 1.0 |
| cell) | | | | fibroblast | | | |
| ionomycin | | | | IL-13 | | | |
| B | 0.0 | 0.0 | 1.3 | Lung | 0.5 | 0.0 | 0.0 |
| lymphocytes | | | | fibroblast | | | |
| PWM | | | | IFN | | | |
| | | | | gamma | | | |
| B | 0.0 | 0.0 | 0.0 | Dermal | 0.0 | 0.0 | 0.0 |
| lymphocytes | | | | fibroblast | | | |
| CD40L | | | | CCD1070 | | | |
| and IL-4 | | | | rest | | | |
| EOL-1 | 0.0 | 0.0 | 0.0 | Dermal | 0.0 | 0.0 | 0.0 |
| dbcAMP | | | | fibroblast | | | |
| | | | | CCD1070 | | | |
| | | | | TNF alpha | | | |
| EOL-1 | 0.0 | 0.0 | 0.0 | Dermal | 0.3 | 0.0 | 0.0 |
| dbcAMP | | | | fibroblast | | | |
| PMA/ionomycin | | | | CCD1070 | | | |
| | | | | IL-1beta | | | |
| Dendritic | 2.1 | 3.5 | 1.1 | Dermal | 0.0 | 0.0 | 0.0 |
| cells none | | | | fibroblast | | | |
| | | | | IFN | | | |
| | | | | gamma | | | |
| Dendritic | 0.0 | 0.0 | 0.0 | Dermal | 0.0 | 0.0 | 0.0 |
| cells LPS | | | | fibroblast | | | |
| | | | | IL-4 | | | |
| Dendritic | 0.0 | 0.0 | 0.0 | IBD | 1.3 | 3.6 | 2.4 |
| cells anti- | | | | Colitis 2 | | | |
| CD40 | | | | | | | |
| Monocytes | 0.0 | 0.0 | 0.0 | IBD | 0.4 | 0.0 | 1.0 |
| rest | | | | Crohn's | | | |
| Monocytes | 0.0 | 0.0 | 0.0 | Colon | 8.1 | 32.3 | 56.6 |
| LPS | | | | | | | |
| Macrophages | 0.0 | 0.0 | 4.8 | Lung | 0.5 | 0.0 | 0.0 |
| rest | | | | | | | |
| Macrophages | 0.0 | 0.0 | 1.8 | Thymus | 100.0 | 100.0 | 100.0 |
| LPS | | | | | | | |
| HUVEC | 0.0 | 0.0 | 0.0 | Kidney | 0.9 | 0.0 | 0.0 |
| none | | | | | | | |
| HUVEC | 0.0 | 0.0 | 1.3 | | | | |
| starved | | | | | | | |

TABLE AYI
Panel 5 Islet

| | Rel. Exp. (%) | | Rel. Exp. (%) |
|---|---|---|---|
| | Ag3027, Run | | Ag3027, Run |
| Tissue Name | 225051163 | Tissue Name | 225051163 |
| 97457_Patient- | 0.0 | 94709_Donor 2 AM - A_adipose | 9.5 |
| 02go_adipose | | | |
| 97476_Patient- | 3.2 | 94710_Donor 2 AM - B_adipose | 0.0 |
| 07sk_skeletal muscle | | | |
| 97477_Patient- | 15.0 | 94711_Donor 2 AM - C_adipose | 5.3 |
| 07ut_uterus | | | |
| 97478_Patient- | 5.4 | 94712_Donor 2 AD - A_adipose | 11.8 |
| 07pl_placenta | | | |
| 99167_Bayer Patient 1 | 0.0 | 94713_Donor 2 AD - B_adipose | 8.7 |
| 97482_Patient- | 0.0 | 94714_Donor 2 AD - C_adipose | 9.3 |
| 08ut_uterus | | | |
| 97483_Patient- | 4.2 | 94742_Donor 3 U - | 0.0 |
| 08pl_placenta | | A_Mesenchymal Stem Cells | |
| 97486_Patient- | 0.0 | 94743_Donor 3 U - | 0.0 |
| 09sk_skeletal muscle | | B_Mesenchymal Stem Cells | |
| 97487_Patient- | 0.0 | 94730_Donor 3 AM - A_adipose | 8.0 |
| 09ut_uterus | | | |
| 97488_Patient- | 0.0 | 94731_Donor 3 AM - B_adipose | 0.0 |
| 09pl_placenta | | | |
| 97492_Patient- | 0.0 | 94732_Donor 3 AM - C_adipose | 8.1 |
| 10ut_uterus | | | |
| 97493_Patient- | 0.0 | 94733_Donor 3 AD - A_adipose | 4.9 |
| 10pl_placenta | | | |
| 97495_Patient- | 8.1 | 94734_Donor 3 AD - B_adipose | 13.3 |
| 11go_adipose | | | |
| 97496_Patient- | 0.0 | 94735_Donor 3 AD - C_adipose | 5.1 |
| 11sk_skeletal muscle | | | |
| 97497_Patient- | 0.0 | 77138_Liver_HepG2untreated | 0.0 |
| 11ut_uterus | | | |
| 97498_Patient- | 0.0 | 73556_Heart_Cardiac stromal | 0.0 |
| 11pl_placenta | | cells (primary) | |
| 97500_Patient- | 3.5 | 81735_Small Intestine | 22.7 |
| 12go_adipose | | | |
| 97501_Patient- | 0.0 | 72409_Kidney_Proximal | 100.0 |
| 12sk_skeletal muscle | | Convoluted Tubule | |
| 97502_Patient- | 0.0 | 82685_Small intestine_Duodenum | 6.1 |
| 12ut_uterus | | | |
| 97503_Patient- | 0.0 | 90650_Adrenal_Adrenocortical | 0.0 |
| 12pl_placenta | | adenoma | |
| 94721_Donor 2 U - | 0.0 | 72410_Kidney_HRCE | 74.7 |
| A_Mesenchymal Stem | | | |
| Cells | | | |
| 94722_Donor 2 U - | 0.0 | 72411_Kidney_HRE | 18.2 |
| B_Mesenchymal Stem | | | |
| Cells | | | |
| 94723_Donor 2 U - | 0.0 | 73139_Uterus_Uterine smooth | 0.0 |
| C_Mesenchymal Stem | | muscle cells | |
| Cells | | | |

General_screening_panel_v1.5 Summary: Ag3027 Expression of the NOV56a gene is highest in a sample derived from a lung cancer (CT=30.5). Significant expression is also seen in samples derived from colon cancer and ovarian cancer. Thus, expression of this gene could be used to differentiate between this sample and other samples on this panel and as a marker to detect the presence of lung, colon, and breast cancers. Furthermore, therapeutic modulation of the expression or function of this gene may be effective in the treatment of lung, colon and breast cancers.

While expression of this gene is seen predominantly in cancer cell lines, significant expression is also seen in fetal kidney (CT=31.8). Furthermore, expression is higher in fetal kidney than in adult kidney (CT=35.8). Thus, expression of this gene could be used to differentiate between adult and fetal kidney. In addition, the expression in fetal kidney suggests that this gene product may be involved in the development of the kidney. Therefore, therapeutic modulation of the expression or function of this gene may be useful in treating disease of the kidney.

Panel 1.2 Summary: Ag1169 Results from one experiment, Run 129128191, with the NOV56a gene are in agreement with Results in Panel 1.3D and General_screening_panel_v1.5. A second run, Run 129656838, produces disparate results.

Panel 1.3D Summary: Ag1169/Ag3027 Two experiments with the same probe and primer both show highest expression of the gene NOV56a in the mammary gland (CTs=31). Low, but significant levels of expression are also seen in a lung cancer cell line. Thus, expression of this gene may be used to differentiate between these samples and other samples on this panel.

Panel 2D Summary: Ag3027 Highest expression of the NOV56a gene is seen in a gastric cancer. Significant expression is also seen in breast cancer, colon cancer and normal kidney. Thus, expression of this gene could be used to differentiate between these samples and other samples on this panel and as a marker for the presence of breast, colon and kidney cancers. A second experiment with the probe/primer set Ag1169 is not included. The amp plot indicates that there were experimental difficulties with this run.

Panel 3D Summary: Ag1169 Expression of the NOV56a gene is restricted to samples derived from lung and gastric cancer cell lines (CTs=33-35). Thus, expression of this gene could be used to differentiate between this sample and other samples on this panel and as a marker to detect the presence of lung and gastric cancers. Furthermore, therapeutic modulation of the expression or function of this gene may be effective in the treatment of lung and gastric cancers.

Panel 4D Summary: Ag1169/Ag3027 Two experiments with the same probe and primer set show expression of the NOV56a gene limited to the thymus (CTs=32-33). Thus, expression of this gene could be used as a marker for thymic tissue. Furthermore, this restricted expression suggests that this gene product may play an important role in T cell development. Therefore, small molecule therapeutics, or antibody therapeutics designed against the protein encoded for by this gene could be utilized to modulate immune function (T cell development) and be important for organ transplant, AIDS treatment or post chemotherapy immune reconstitution.

Panel 5 Islet Summary: Ag3027 Expression of the NOV56a gene is restricted to a sample derived from the kidney (CT=34.9). This expression is consistent with expression in Panel 1.3D. Thus, expression of this gene could be used as a marker for kidney tissue.

NOV57

Expression of gene NOV57 was assessed using the primer-probe sets Ag3031, Ag1301b and Ag1415, described in Tables AZA, AZB and AZC. Results of the RTQ-PCR runs are shown in Tables AZD, AZE, AZF and AZG.

TABLE AZA
Probe Name Ag3031

| | | | Start | |
|---|---|---|---|---|
| Primers | Sequences | Length | Position | SEQ ID NO: |
| Forward | 5′-aaaaggtgatgtctggagcat-3′ | 21 | 616 | 1216 |
| Probe | TET-5′-tgtatgtcatgctctgtgccagccta-3′-TAMRA | 26 | 648 | 1217 |
| Reverse | 5′-gatgtctgtgtcgtcaaaagg-3′ | 21 | 674 | 1218 |

TABLE AZB
Probe Name Ag1301b

| | | | Start | |
|---|---|---|---|---|
| Primers | Sequences | Length | Position | SEQ ID NO: |
| Forward | 5′-aaaaggtgatgtctggagcat-3′ | 21 | 616 | 1219 |
| Probe | TET-5′-tgtatgtcatgctctgtgccagccta-3′-TAMRA | 26 | 648 | 1220 |
| Reverse | 5′-gatgtctgtgtcgtcaaaagg-3′ | 21 | 674 | 1221 |

TABLE AZC
Probe Name Ag1415

| | | | Start | |
|---|---|---|---|---|
| Primers | Sequences | Length | Position | SEQ ID NO: |
| Forward | 5′-aaaaggtgatgtctggagcat-3′ | 21 | 616 | 1222 |
| Probe | TET-5′-tgtatgtcatgctctgtgccagccta-3′-TAMRA | 26 | 648 | 1223 |
| Reverse | 5′-gatgtctgtgtcgtcaaaagg-3′ | 21 | 674 | 1224 |

TABLE AZD
CNS_neurodegeneration_v1.0

| | Rel. Exp. (%) | Rel. Exp. (%) | | Rel. Exp. (%) | Rel. Exp. (%) |
|---|---|---|---|---|---|
| | Ag3031, Run | Ag3031, Run | Tissue | Ag3031, Run | Ag3031, Run |
| Tissue Name | 211011868 | 225437445 | Name | 211011868 | 225437445 |
| AD 1 Hippo | 23.8 | 46.7 | Control | 6.0 | 10.2 |
| | | | (Path) 3 | | |
| | | | Temporal | | |
| | | | Ctx | | |
| AD 2 Hippo | 21.2 | 21.9 | Control | 26.4 | 28.1 |
| | | | (Path) 4 | | |
| | | | Temporal | | |
| | | | Ctx | | |
| AD 3 Hippo | 14.2 | 11.6 | AD 1 | 31.9 | 41.8 |
| | | | Occipital | | |
| | | | Ctx | | |
| AD 4 Hippo | 7.4 | 10.5 | AD 2 | 0.0 | 0.0 |
| | | | Occipital | | |
| | | | Ctx | | |
| | | | (Missing) | | |
| AD 5 hippo | 94.0 | 94.0 | AD 3 | 17.9 | 17.4 |
| | | | Occipital | | |
| | | | Ctx | | |
| AD 6 Hippo | 53.6 | 61.6 | AD 4 | 12.9 | 17.3 |
| | | | Occipital | | |
| | | | Ctx | | |
| Control 2 | 12.7 | 17.0 | AD 5 | 35.1 | 20.9 |
| Hippo | | | Occipital | | |
| | | | Ctx | | |
| Control 4 | 14.6 | 15.2 | AD 6 | 20.9 | 27.5 |
| Hippo | | | Occipital | | |
| | | | Ctx | | |
| Control (Path) | 22.2 | 7.2 | Control 1 | 4.9 | 4.9 |
| 3 Hippo | | | Occipital | | |
| | | | Ctx | | |
| AD 1 Temporal | 42.3 | 42.9 | Control 2 | 46.7 | 32.1 |
| Ctx | | | Occipital | | |
| | | | Ctx | | |
| AD 2 Temporal | 78.5 | 29.9 | Control 3 | 17.8 | 19.2 |
| Ctx | | | Occipital | | |
| | | | Ctx | | |
| AD 3 Temporal | 17.9 | 20.4 | Control 4 | 13.2 | 12.1 |
| Ctx | | | Occipital | | |
| | | | Ctx | | |
| AD 4 Temporal | 30.1 | 31.2 | Control | 49.3 | 59.9 |
| Ctx | | | (Path) 1 | | |
| | | | Occipital | | |
| | | | Ctx | | |
| AD 5 Inf | 100.0 | 94.6 | Control | 13.6 | 8.8 |
| Temporal Ctx | | | (Path) 2 | | |
| | | | Occipital | | |
| | | | Ctx | | |
| AD 5 | 59.9 | 52.5 | Control | 6.1 | 4.7 |
| SupTemporal | | | (Path) 3 | | |
| Ctx | | | Occipital | | |
| | | | Ctx | | |
| AD 6 Inf | 75.3 | 95.9 | Control | 31.2 | 21.2 |
| Temporal Ctx | | | (Path) 4 | | |
| | | | Occipital | | |
| | | | Ctx | | |
| AD 6 Sup | 94.6 | 100.0 | Control 1 | 10.3 | 11.1 |
| Temporal Ctx | | | Parietal Ctx | | |
| Control 1 | 8.5 | 7.3 | Control 2 | 56.6 | 60.7 |
| Temporal Ctx | | | Parietal Ctx | | |
| Control 2 | 16.3 | 18.8 | Control 3 | 15.4 | 15.0 |
| Temporal Ctx | | | Parietal Ctx | | |
| Control 3 | 13.0 | 15.3 | Control | 34.2 | 41.8 |
| Temporal Ctx | | | (Path) 1 | | |
| | | | Parietal Ctx | | |
| Control 4 | 12.8 | 16.6 | Control | 15.4 | 20.3 |
| Temporal Ctx | | | (Path) 2 | | |
| | | | Parietal Ctx | | |
| Control (Path) | 26.8 | 31.0 | Control | 8.4 | 7.0 |
| 1 Temporal Ctx | | | (Path) 3 | | |
| | | | Parietal Ctx | | |
| Control (Path) | 25.5 | 17.8 | Control | 44.1 | 31.6 |
| 2 Temporal Ctx | | | (Path) 4 | | |
| | | | Parietal Ctx | | |

TABLE AZE
Panel 1.3D

| | Rel. Exp. (%) | Rel. Exp. (%) | | Rel. Exp. (%) | Rel. Exp. (%) |
|---|---|---|---|---|---|
| | Ag1301b, Run | Ag3031, Run | | Ag1301b, Run | Ag3031, Run |
| Tissue Name | 165528224 | 167961982 | Tissue Name | 165528224 | 167961982 |
| Liver | 18.4 | 32.3 | Kidney (fetal) | 10.7 | 66.0 |
| adenocarcinoma | | | | | |
| Pancreas | 4.7 | 11.9 | Renal ca. 786-0 | 15.3 | 25.5 |
| Pancreatic ca. | 4.9 | 4.9 | Renal ca. | 11.9 | 12.5 |
| CAPAN 2 | | | A498 | | |
| Adrenal gland | 23.0 | 11.0 | Renal ca. RXF | 14.9 | 18.0 |
| | | | 393 | | |
| Thyroid | 7.1 | 8.5 | Renal ca. | 8.0 | 10.5 |
| | | | ACHN | | |
| Salivary gland | 10.1 | 13.7 | Renal ca. UO- | 6.0 | 1.1 |
| | | | 31 | | |
| Pituitary gland | 18.2 | 9.2 | Renal ca. TK- | 7.4 | 18.6 |
| | | | 10 | | |
| Brain (fetal) | 24.0 | 42.3 | Liver | 11.9 | 17.8 |
| Brain (whole) | 43.8 | 19.6 | Liver (fetal) | 20.6 | 14.7 |
| Brain (amygdala) | 20.4 | 9.6 | Liver ca. | 22.2 | 28.7 |
| | | | (hepatoblast) | | |
| | | | HepG2 | | |
| Brain (cerebellum) | 50.7 | 50.3 | Lung | 17.0 | 22.8 |
| Brain | 18.7 | 8.1 | Lung (fetal) | 10.4 | 26.4 |
| (hippocampus) | | | | | |
| Brain (substantia | 8.4 | 8.9 | Lung ca. | 9.1 | 17.4 |
| nigra) | | | (small cell) | | |
| | | | LX-1 | | |
| Brain (thalamus) | 20.0 | 10.6 | Lung ca. | 1.5 | 4.0 |
| | | | (small cell) | | |
| | | | NCI-H69 | | |
| Cerebral Cortex | 5.3 | 7.2 | Lung ca. | 18.4 | 57.4 |
| | | | (s.cell var.) | | |
| | | | SHP-77 | | |
| Spinal cord | 9.6 | 7.0 | Lung ca. | 50.3 | 4.2 |
| | | | (large | | |
| | | | cell)NCI-H460 | | |
| glio/astro U87-MG | 12.1 | 16.5 | Lung ca. (non- | 8.0 | 28.1 |
| | | | sm. cell) A549 | | |
| glio/astro U-118- | 15.5 | 13.4 | Lung ca. (non- | 10.2 | 18.2 |
| MG | | | s.cell) NCI- | | |
| | | | H23 | | |
| astrocytoma | 6.9 | 11.1 | Lung ca. (non- | 20.3 | 33.0 |
| SW1783 | | | s.cell) HOP-62 | | |
| neuro*; met SK-N- | 11.4 | 10.9 | Lung ca. (non- | 9.7 | 23.5 |
| AS | | | s.cl) NCI- | | |
| | | | H522 | | |
| astrocytoma SF- | 12.5 | 25.5 | Lung ca. | 4.0 | 8.5 |
| 539 | | | (squam.) SW | | |
| | | | 900 | | |
| astrocytoma SNB- | 10.9 | 18.9 | Lung ca. | 1.6 | 4.8 |
| 75 | | | (squam.) NCI- | | |
| | | | H596 | | |
| glioma SNB-19 | 32.1 | 25.3 | Mammary | 36.9 | 22.5 |
| | | | gland | | |
| glioma U251 | 100.0 | 100.0 | Breast ca.* | 5.4 | 6.7 |
| | | | (pl.ef) MCF-7 | | |
| glioma SF-295 | 18.4 | 46.0 | Breast ca.* | 16.7 | 7.9 |
| | | | (pl.ef) MDA- | | |
| | | | MB-231 | | |
| Heart (fetal) | 2.3 | 6.2 | Breast ca.* | 13.2 | 46.7 |
| | | | (pl.ef) T47D | | |
| Heart | 8.4 | 8.2 | Breast ca. BT- | 9.9 | 6.6 |
| | | | 549 | | |
| Skeletal muscle | 6.3 | 24.1 | Breast ca. | 1.6 | 11.9 |
| (fetal) | | | MDA-N | | |
| Skeletal muscle | 20.2 | 7.9 | Ovary | 3.0 | 5.1 |
| Bone marrow | 21.8 | 25.2 | Ovarian ca. | 5.1 | 12.0 |
| | | | OVCAR-3 | | |
| Thymus | 18.2 | 38.4 | Ovarian ca. | 4.7 | 8.8 |
| | | | OVCAR-4 | | |
| Spleen | 26.6 | 14.0 | Ovarian ca. | 8.3 | 42.3 |
| | | | OVCAR-5 | | |
| Lymph node | 42.6 | 23.7 | Ovarian ca. | 2.8 | 0.9 |
| | | | OVCAR-8 | | |
| Colorectal | 16.8 | 16.8 | Ovarian ca. | 2.6 | 11.7 |
| | | | IGROV-1 | | |
| Stomach | 37.4 | 5.9 | Ovarian ca.* | 10.0 | 29.9 |
| | | | (ascites) SK- | | |
| | | | OV-3 | | |
| Small intestine | 36.6 | 14.2 | Uterus | 38.4 | 17.4 |
| Colon ca. SW480 | 3.7 | 4.5 | Placenta | 11.1 | 9.4 |
| Colon ca.* | 3.8 | 24.0 | Prostate | 18.6 | 8.5 |
| SW620(SW480 | | | | | |
| met) | | | | | |
| Colon ca. HT29 | 1.6 | 4.6 | Prostate ca.* | 6.9 | 14.1 |
| | | | (bone met) PC-3 | | |
| Colon ca. HCT- | 3.0 | 9.9 | Testis | 45.1 | 26.6 |
| 116 | | | | | |
| Colon ca. CaCo-2 | 2.1 | 5.1 | Melanoma | 0.8 | 1.2 |
| | | | Hs688 (A).T | | |
| Colon ca. | 5.4 | 3.6 | Melanoma* | 3.8 | 3.4 |
| tissue (ODO3866) | | | (met) | | |
| | | | Hs688(B).T | | |
| Colon ca. HCC- | 6.2 | 15.6 | Melanoma | 4.6 | 8.0 |
| 2998 | | | UACC-62 | | |
| Gastric ca.* (liver | 20.0 | 13.3 | Melanoma | 28.5 | 9.9 |
| met) NCI-N87 | | | M14 | | |
| Bladder | 8.4 | 12.2 | Melanoma | 0.7 | 8.1 |
| | | | LOX IMVI | | |
| Trachea | 13.6 | 6.4 | Melanoma* | 2.2 | 2.9 |
| | | | (met) SK- | | |
| | | | MEL-5 | | |
| Kidney | 10.4 | 23.8 | Adipose | 8.1 | 12.3 |

TABLE AZF
Panel 2.2

| | Rel. Exp. (%) | | Rel. Exp. (%) |
|---|---|---|---|
| | Ag1301b, Run | | Ag1301b, Run |
| Tissue Name | 173859869 | Tissue Name | 173859869 |
| Normal Colon | 39.2 | Kidney Margin | 79.0 |
| | | (OD04348) | |
| Colon cancer | 6.3 | Kidney malignant | 6.0 |
| (OD06064) | | cancer (OD06204B) | |
| Colon Margin | 13.0 | Kidney normal adjacent | 15.4 |
| (OD06064) | | tissue (OD06204E) | |
| Colon cancer | 0.0 | Kidney Cancer | 24.5 |
| (OD06159) | | (OD04450-01) | |
| Colon Margin | 27.0 | Kidney Margin | 23.7 |
| (OD06159) | | (OD04450-03) | |
| Colon cancer | 2.5 | Kidney Cancer | 3.0 |
| (OD06297-04) | | 8120613 | |
| Colon Margin | 39.8 | Kidney Margin | 38.2 |
| (OD06297-015) | | 8120614 | |
| CC Gr.2 ascend colon | 3.8 | Kidney Cancer | 8.0 |
| (ODO3921) | | 9010320 | |
| CC Margin (ODO3921) | 4.4 | Kidney Margin | 10.8 |
| | | 9010321 | |
| Colon cancer metastasis | 14.3 | Kidney Cancer | 15.2 |
| (OD06104) | | 8120607 | |
| Lung Margin | 17.2 | Kidney Margin | 3.8 |
| (OD06104) | | 8120608 | |
| Colon mets to lung | 0.0 | Normal Uterus | 33.7 |
| (OD04451-01) | | | |
| Lung Margin | 10.2 | Uterine Cancer 064011 | 33.9 |
| (OD04451-02) | | | |
| Normal Prostate | 30.6 | Normal Thyroid | 2.9 |
| Prostate Cancer | 7.6 | Thyroid Cancer 064010 | 4.4 |
| (OD04410) | | | |
| Prostate Margin | 25.5 | Thyroid Cancer | 29.9 |
| (OD04410) | | A302152 | |
| Normal Ovary | 25.0 | Thyroid Margin | 7.3 |
| | | A302153 | |
| Ovarian cancer | 8.1 | Normal Breast | 37.6 |
| (OD06283-03) | | | |
| Ovarian Margin | 34.4 | Breast Cancer | 21.6 |
| (OD06283-07) | | (OD04566) | |
| Ovarian Cancer 064008 | 46.3 | Breast Cancer 1024 | 100.0 |
| Ovarian cancer | 34.4 | Breast Cancer | 25.2 |
| (OD06145) | | (OD04590-01) | |
| Ovarian Margin | 52.9 | Breast Cancer Mets | 35.6 |
| (OD06145) | | (OD04590-03) | |
| Ovarian cancer | 11.6 | Breast Cancer | 45.1 |
| (OD06455-03) | | Metastasis (OD04655- | |
| | | 05) | |
| Ovarian Margin | 18.8 | Breast Cancer 064006 | 21.2 |
| (OD06455-07) | | | |
| Normal Lung | 22.8 | Breast Cancer 9100266 | 26.6 |
| Invasive poor diff. lung | 18.7 | Breast Margin 9100265 | 19.3 |
| adeno (ODO4945-01) | | | |
| Lung Margin | 13.9 | Breast Cancer A209073 | 6.1 |
| (ODO4945-03) | | | |
| Lung Malignant Cancer | 11.7 | Breast Margin | 35.8 |
| (OD03126) | | A2090734 | |
| Lung Margin | 12.2 | Breast cancer | 49.3 |
| (OD03126) | | (OD06083) | |
| Lung Cancer | 9.0 | Breast cancer node | 25.9 |
| (OD05014A) | | metastasis (OD06083) | |
| Lung Margin | 35.4 | Normal Liver | 36.3 |
| (OD05014B) | | | |
| Lung cancer (OD06081) | 23.7 | Liver Cancer 1026 | 2.5 |
| Lung Margin | 27.9 | Liver Cancer 1025 | 45.7 |
| (OD06081) | | | |
| Lung Cancer | 13.3 | Liver Cancer 6004-T | 30.1 |
| (OD04237-01) | | | |
| Lung Margin | 28.5 | Liver Tissue 6004-N | 27.7 |
| (OD04237-02) | | | |
| Ocular Melanoma | 11.5 | Liver Cancer 6005-T | 6.7 |
| Metastasis | | | |
| Ocular Melanoma | 27.0 | Liver Tissue 6005-N | 17.4 |
| Margin (Liver) | | | |
| Melanoma Metastasis | 21.6 | Liver Cancer 064003 | 32.3 |
| Melanoma Margin | 14.7 | Normal Bladder | 13.2 |
| (Lung) | | | |
| Normal Kidney | 17.8 | Bladder Cancer 1023 | 23.0 |
| Kidney Ca, Nuclear | 50.3 | Bladder Cancer | 20.0 |
| grade 2 (OD04338) | | A302173 | |
| Kidney Margin | 24.3 | Normal Stomach | 89.5 |
| (OD04338) | | | |
| Kidney Ca Nuclear | 62.0 | Gastric Cancer | 5.7 |
| grade 1/2 (OD04339) | | 9060397 | |
| Kidney Margin | 16.3 | Stomach Margin | 17.7 |
| (OD04339) | | 9060396 | |
| Kidney Ca, Clear cell | 7.3 | Gastric Cancer | 19.6 |
| type (OD04340) | | 9060395 | |
| Kidney Margin | 10.7 | Stomach Margin 9060394 | 42.6 |
| (OD04340) | | | |
| Kidney Ca, Nuclear | 4.6 | Gastric Cancer 064005 | 7.5 |
| grade 3 (OD04348) | | | |

TABLE AZG
Panel 4D

| | Rel. | Rel. | Rel. | | Rel. | Rel. | Rel. |
|---|---|---|---|---|---|---|---|
| | Exp. (%) | Exp. (%) | Exp. (%) | | Exp. (%) | Exp. (%) | Exp. (%) |
| | Ag1301b, | Ag1415, | Ag3031, | | Ag1301b, | Ag1415, | Ag3031, |
| Tissue | Run | Run | Run | Tissue | Run | Run | Run |
| Name | 138983163 | 138642033 | 162426783 | Name | 138983163 | 138642033 | 162426783 |
| Secondary | 17.0 | 25.3 | 13.8 | HUVEC | 11.5 | 13.0 | 11.3 |
| Th1 act | | | | IL-1beta | | | |
| Secondary | 22.7 | 18.9 | 20.0 | HUVEC | 28.3 | 24.7 | 20.2 |
| Th2 act | | | | IFN | | | |
| | | | | gamma | | | |
| Secondary | 32.3 | 26.4 | 20.0 | HUVEC | 3.2 | 6.6 | 12.3 |
| Tr1 act | | | | TNF | | | |
| | | | | alpha + IFN | | | |
| | | | | gamma | | | |
| Secondary | 22.2 | 20.3 | 12.0 | HUVEC | 11.7 | 9.7 | 9.7 |
| Th1 rest | | | | TNF | | | |
| | | | | alpha + IL4 | | | |
| Secondary | 42.6 | 37.9 | 21.0 | HUVEC | 10.1 | 8.2 | 11.6 |
| Th2 rest | | | | IL-11 | | | |
| Secondary | 27.9 | 27.5 | 26.8 | Lung | 33.9 | 28.7 | 25.2 |
| Tr1 rest | | | | Microvascular | | | |
| | | | | EC | | | |
| | | | | none | | | |
| Primary | 33.4 | 29.9 | 19.1 | Lung | 23.2 | 19.1 | 24.8 |
| Th1 act | | | | Microvascular | | | |
| | | | | EC | | | |
| | | | | TNF | | | |
| | | | | alpha + IL-1beta | | | |
| Primary | 28.1 | 41.2 | 13.3 | Microvascular | 41.8 | 45.1 | 25.7 |
| Th2 act | | | | Dermal EC | | | |
| | | | | none | | | |
| Primary | 49.0 | 52.5 | 20.9 | Microsvasular | 27.5 | 40.3 | 22.4 |
| Tr1 act | | | | Dermal EC | | | |
| | | | | TNF | | | |
| | | | | alpha + IL-1beta | | | |
| Primary | 80.7 | 87.1 | 80.7 | Bronchial | 20.3 | 32.3 | 22.1 |
| Th1 rest | | | | epithelium | | | |
| | | | | TNF | | | |
| | | | | alpha + IL1beta | | | |
| Primary | 67.4 | 68.8 | 64.2 | Small | 7.3 | 3.3 | 4.8 |
| Th2 rest | | | | airway | | | |
| | | | | epithelium | | | |
| | | | | none | | | |
| Primary | 46.0 | 50.3 | 54.7 | Small | 34.4 | 35.1 | 26.8 |
| Tr1 rest | | | | airway | | | |
| | | | | epithelium | | | |
| | | | | TNF | | | |
| | | | | alpha + IL-1beta | | | |
| CD45RA | 12.9 | 8.0 | 16.5 | Coronery | 7.9 | 7.9 | 12.3 |
| CD4 | | | | artery | | | |
| lymphocyte | | | | SMC rest | | | |
| act | | | | | | | |
| CD45RO | 33.4 | 44.8 | 22.4 | Coronery | 7.6 | 10.7 | 8.1 |
| CD4 | | | | artery | | | |
| lymphocyte | | | | SMC | | | |
| act | | | | TNF | | | |
| | | | | alpha + IL-1beta | | | |
| CD8 | 22.8 | 23.0 | 18.4 | Astrocytes | 7.9 | 10.3 | 12.7 |
| lymphocyte | | | | rest | | | |
| act | | | | | | | |
| Secondary | 22.2 | 25.5 | 25.0 | Astrocytes | 8.7 | 5.8 | 13.3 |
| CD8 | | | | TNF | | | |
| lymphocyte | | | | alpha + IL-1beta | | | |
| rest | | | | | | | |
| Secondary | 19.3 | 21.8 | 22.5 | KU-812 | 40.1 | 35.4 | 48.0 |
| CD8 | | | | (Basophil) | | | |
| lymphocyte | | | | rest | | | |
| act | | | | | | | |
| CD4 | 41.5 | 42.3 | 34.6 | KU-812 | 57.8 | 61.1 | 78.5 |
| lymphocyte | | | | (Basophil) | | | |
| none | | | | PMA/ionomycin | | | |
| 2ry | 65.1 | 54.7 | 41.2 | CCD1106 | 3.8 | 8.0 | 6.6 |
| Th1/Th2/Tr1_{—} | | | | (Keratinocytes) | | | |
| anti- | | | | none | | | |
| CD95 | | | | | | | |
| CH11 | | | | | | | |
| LAK cells | 28.7 | 37.1 | 32.3 | CCD1106 | 27.4 | 24.5 | 5.3 |
| rest | | | | (Keratinocytes) | | | |
| | | | | TNF | | | |
| | | | | alpha + IL-1beta | | | |
| LAK cells | 38.7 | 49.0 | 36.3 | Liver | 17.0 | 9.4 | 5.0 |
| IL-2 | | | | cirrhosis | | | |
| LAK cells | 26.8 | 27.4 | 26.8 | Lupus | 24.1 | 23.8 | 9.5 |
| IL-2 + IL- | | | | kidney | | | |
| 12 | | | | | | | |
| LAK cells | 43.5 | 45.4 | 42.9 | NCI-H292 | 38.7 | 49.3 | 45.4 |
| IL-2 + IFN | | | | none | | | |
| gamma | | | | | | | |
| LAK cells | 26.2 | 25.2 | 35.6 | NCI-H292 | 58.6 | 51.4 | 46.7 |
| IL-2 + IL- | | | | IL-4 | | | |
| 18 | | | | | | | |
| LAK cells | 8.3 | 8.6 | 3.3 | NCI-H292 | 56.3 | 46.0 | 54.3 |
| PMA/ionomycin | | | | IL-9 | | | |
| NK Cells | 28.7 | 35.8 | 32.8 | NCI-H292 | 30.4 | 31.9 | 23.3 |
| IL-2 rest | | | | IL-13 | | | |
| Two Way | 42.3 | 49.0 | 46.3 | NCI-H292 | 16.7 | 28.3 | 29.1 |
| MLR 3 | | | | IFN | | | |
| day | | | | gamma | | | |
| Two Way | 17.9 | 16.2 | 13.5 | HPAEC | 15.7 | 26.4 | 19.1 |
| MLR 5 | | | | none | | | |
| day | | | | | | | |
| Two Way | 14.7 | 12.6 | 14.4 | HPAEC | 23.8 | 32.5 | 27.9 |
| MLR 7 | | | | TNF | | | |
| day | | | | alpha + IL-1beta | | | |
| PBMC rest | 21.9 | 29.9 | 19.2 | Lung | 13.0 | 11.0 | 11.9 |
| | | | | fibroblast | | | |
| | | | | none | | | |
| PBMC | 66.9 | 53.2 | 50.3 | Lung | 8.7 | 7.2 | 13.2 |
| PWM | | | | fibroblast | | | |
| | | | | TNF | | | |
| | | | | alpha + IL-1beta | | | |
| PBMC | 35.6 | 46.7 | 23.3 | Lung | 5.6 | 12.0 | 10.5 |
| PHA-L | | | | fibroblast | | | |
| | | | | IL-4 | | | |
| Ramos (B | 23.5 | 33.2 | 16.5 | Lung | 8.5 | 7.4 | 15.3 |
| cell) none | | | | fibroblast | | | |
| | | | | IL-9 | | | |
| Ramos (B | 53.2 | 53.2 | 49.3 | Lung | 20.3 | 16.3 | 8.9 |
| cell) | | | | fibroblast | | | |
| ionomycin | | | | IL-13 | | | |
| B | 29.9 | 36.3 | 34.2 | Lung | 11.1 | 10.4 | 13.0 |
| lymphocytes | | | | fibroblast | | | |
| PWM | | | | IFN | | | |
| | | | | gamma | | | |
| B | 33.2 | 36.6 | 35.8 | Dermal | 47.0 | 11.3 | 13.8 |
| lymphocytes | | | | fibroblast | | | |
| CD40L | | | | CCD1070 | | | |
| and IL-4 | | | | rest | | | |
| EOL-1 | 18.9 | 14.8 | 12.8 | Dermal | 45.7 | 53.6 | 55.1 |
| dbcAMP | | | | fibroblast | | | |
| | | | | CCD1070 | | | |
| | | | | TNF alpha | | | |
| EOL-1 | 30.4 | 29.1 | 25.5 | Dermal | 16.8 | 17.3 | 15.4 |
| dbcAMP | | | | fibroblast | | | |
| PMA/ionomycin | | | | CCD1070 | | | |
| | | | | IL-1beta | | | |
| Dendritic | 14.9 | 10.0 | 13.7 | Dermal | 5.8 | 8.8 | 6.5 |
| cells none | | | | fibroblast | | | |
| | | | | IFN | | | |
| | | | | gamma | | | |
| Dendritic | 15.7 | 5.8 | 8.7 | Dermal | 17.6 | 20.0 | 13.6 |
| cells LPS | | | | fibroblast | | | |
| | | | | IL-4 | | | |
| Dendritic | 12.7 | 17.7 | 15.2 | IBD | 2.0 | 1.3 | 1.1 |
| cells anti- | | | | Colitis 2 | | | |
| CD40 | | | | | | | |
| Monocytes | 35.6 | 27.5 | 36.6 | IBD | 3.0 | 1.4 | 1.4 |
| rest | | | | Crohn's | | | |
| Monocytes | 34.6 | 43.8 | 25.0 | Colon | 33.0 | 26.2 | 34.6 |
| LPS | | | | | | | |
| Macrophages | 19.2 | 16.6 | 17.2 | Lung | 6.8 | 13.5 | 7.5 |
| rest | | | | | | | |
| Macrophages | 17.9 | 16.2 | 6.7 | Thymus | 100.0 | 100.0 | 55.5 |
| LPS | | | | | | | |
| HUVEC | 9.8 | 13.2 | 17.3 | Kidney | 87.1 | 79.0 | 100.0 |
| none | | | | | | | |
| HUVEC | 27.2 | 27.5 | 28.3 | | | | |
| starved | | | | | | | |

CNS_neurodegeneration_v1.0 Summary: Ag3031 Two experiments with the same probe and primer set produce results that are in excellent agreement. The NOV57 gene, a kinase homolog, is expressed more highly in the temporal cortex of brains from Alzheimer's disease patients than in the temporal cortex of normal brains unaffected by Alzheimer's disease. Kinases have been shown to play a role in the pathogenesis of Alzheimer's disease. The dysregulation of this kinase, NOV57, indicates an active role for this pathway in disease pathogenesis. Thus, inhibitors of this gene product, by modulating this pathway, may have utility in the treatment of Alzheimer's disease and other neurodegenerative diseases.

References:

Morishima Y, Gotoh Y, Zieg J, Barrett T, Takano H, Flavell R, Davis R J, Shirasaki Y, Greenberg M E. Beta-amyloid induces neuronal apoptosis via a mechanism that involves the c-Jun N-terminal kinase pathway and the induction of Fas ligand. J Neurosci Oct. 1, 2001;21(19):7551-60

Elevated levels of beta-Amyloid (Abeta) are present in the brains of individuals with either the sporadic or familial form of Alzheimer's disease (AD), and the deposition of Abeta within the senile plaques that are a hallmark of AD is thought to be a primary cause of the cognitive dysfunction that occurs in AD. Recent evidence suggests that Abeta induces neuronal apoptosis in the brain and in primary neuronal cultures, and that this Abeta-induced neuronal death may be responsible in part for the cognitive decline found in AD patients. In this study we have characterized one mechanism by which Abeta induces neuronal death. We found that in cortical neurons exposed to Abeta, activated c-Jun N-terminal kinase (JNK) is required for the phosphorylation and activation of the c-Jun transcription factor, which in turn stimulates the transcription of several key target genes, including the death inducer Fas ligand. The binding of Fas ligand to its receptor Fas then induces a cascade of events that lead to caspase activation and ultimately cell death. By analyzing the effects of mutations in each of the components of the JNK-c-Jun-Fas ligand-Fas pathway, we demonstrate that this pathway plays a critical role in mediating Abeta-induced death of cultured neurons. These findings raise the possibility that the JNK pathway may also contribute to Abeta-dependent death in AD patients

Panel 1.3D Summary: Ag1301b/Ag3031 Two experiments with the same probe and primer set produce results that are in excellent agreement, with highest expression of the NOV57 gene in a brain cancer cell line (CTs=29-30). Overall, this gene is expressed at moderate to low levels in all the samples in this panel.

This gene has low to moderate expression in several endocrine/metabolic-related tissues, including adipose, pancreas, liver, skeletal muscle and thyroid. Thus, a therapeutic modulator to this gene and/or gene-product may be useful in the treatment of diseases which affect the endocrine system.

Panel 2.2 Summary: Ag1301b The NOV57 gene is expressed in breast cancer at a moderate level. It is also expressed at a higher level in normal gastric, prostate and colon tissues compared to the adjacent tumors. Hence, inhibition of this drug might be used for treatment of breast cancer. It could also be used as a diagnostic marker for gastric, prostate and colon cancers.

Panel 4D Summary: Ag1301b/Ag1415/Ag3031 Three experiments with the same probe and primer sets produce results that are in excellent agreement, with highest expression of the NOV57 gene in the thymus and kidney. This gene is also expressed at higher levels in resting Th1 and Th2 lymphocytes than in activated Th1 and Th2 lymphocytes. Therefore, small molecule agonists of the gene product may be useful as therapeutics to reduce the activation of Th1 and Th2 cells and thus reduce symptoms in patients with autoimmune and inflammatory diseases, such as Crohn's disease, ulcerative colitis, multiple sclerosis, chronic obstructive pulmonary disease, asthma, emphysema, rheumatoid arthritis, lupus erythematosus, or psoriasis.

NOV58a and NOV58b: Gap Junction Beta-5 (connexin)

Expression of gene NOV58a and variant NOV58b was assessed using the primer-probe set Ag2914, described in Table BAA.

TABLE BAA
Probe Name Ag2914

| | | | Start | |
|---|---|---|---|---|
| Primers | Sequences | Length | Position | SEQ ID NO: |
| Forward | 5′-aacactgtggactgcttcatct-3′ | 22 | 517 | 1225 |
| Probe | TET-5′-ccaaacccactgagaagacgatcttca-3′-TAMRA | 27 | 539 | 1226 |
| Reverse | 5′-atacacaagcatgaggtgatga-3′ | 22 | 578 | 1227 |

CNS_neurodegeneration_v1.0 Summary: Ag2914 The amp plot indicates that there are experimental difficulties with this run (data not shown).

Panel 1.3D Summary: Ag2914 Expression of this gene is low/undetectable (CTs>35) across all of the samples on this panel (data not shown).

Panel 2D Summary: Ag2914 Expression of this gene is low/undetectable (CTs>35) across all of the samples on this panel (data not shown).

Panel 4D Summary: Ag2914 The amp plot indicates that there are experimental difficulties with this run (data not shown).

### BB. CG56633-01: Translation Initiation Factor 5

Expression of gene CG56633-01 was assessed using the primer-probe set Ag2900, described in Table BBA. Results of the RTQ-PCR runs are shown in Tables BBB, BBC, BBD and BBE.

TABLE BBA
Probe Name Ag2900

| | | | Start | |
|---|---|---|---|---|
| Primers | Sequences | Length | Position | SEQ ID NO: |
| Forward | 5′-gctaagttccttgatgcttctg-3′ | 22 | 184 | 1228 |
| Probe | TET-5′-caaaacttgattaccgtcgatgtgca-3′-TAMRA | 26 | 209 | 1229 |
| Reverse | 5′-ccaccagaatgtcaaagagtgt-3′ | 22 | 238 | 1230 |

TABLE BBB
CNS_neurodegeneration_v1.0

| | Rel. Exp. (%) Ag2900, | | Rel. Exp. (%) Ag2900, |
|---|---|---|---|
| Tissue Name | Run 206485415 | Tissue Name | Run 206485415 |
| AD 1 Hippo | 12.9 | Control (Path) 3 | 7.0 |
| | | Temporal Ctx | |
| AD 2 Hippo | 33.9 | Control (Path) 4 | 18.6 |
| | | Temporal Ctx | |
| AD 3 Hippo | 7.4 | AD 1 Occipital Ctx | 14.5 |
| AD 4 Hippo | 11.2 | AD 2 Occipital Ctx | 0.0 |
| | | (Missing) | |
| AD 5 Hippo | 68.8 | AD 3 Occipital Ctx | 8.0 |
| AD 6 Hippo | 61.6 | AD 4 Occipital Ctx | 16.6 |
| Control 2 Hippo | 36.1 | AD 5 Occipital Ctx | 36.3 |
| Control 4 Hippo | 21.0 | AD 6 Occipital Ctx | 25.7 |
| Control (Path) 3 | 7.7 | Control 1 Occipital | 6.8 |
| Hippo | | Ctx | |
| AD 1 Temporal Ctx | 19.6 | Control 2 Occipital | 65.5 |
| | | Ctx | |
| AD 2 Temporal Ctx | 27.5 | Control 3 Occipital | 14.6 |
| | | Ctx | |
| AD 3 Temporal Ctx | 6.2 | Control 4 Occipital | 12.3 |
| | | Ctx | |
| AD 4 Temporal Ctx | 17.6 | Control (Path) 1 | 100.0 |
| | | Occipital Ctx | |
| AD 5 Inf Temporal | 59.6 | Control (Path) 2 | 6.5 |
| Ctx | | Occipital Ctx | |
| AD 5 Sup Temporal | 42.0 | Control (Path) 3 | 5.1 |
| Ctx | | Occipital Ctx | |
| AD 6 Inf Temporal | 65.1 | Control (Path) 4 | 13.2 |
| Ctx | | Occipital Ctx | |
| AD 6 Sup Temporal | 48.3 | Control 1 Parietal | 8.0 |
| Ctx | | Ctx | |
| Control 1 Temporal | 8.0 | Control 2 Parietal | 24.5 |
| Ctx | | Ctx | |
| Control 2 Temporal | 52.9 | Control 3 Parietal | 18.3 |
| Ctx | | Ctx | |
| Control 3 Temporal | 18.2 | Control (Path) 1 | 86.5 |
| Ctx | | Parietal Ctx | |
| Control 3 Temporal | 9.0 | Control (Path) 2 | 17.4 |
| Ctx | | Parietal Ctx | |
| Control (Path) 1 | 57.0 | Control (Path) 3 | 9.4 |
| Temporal Ctx | | Parietal Ctx | |
| Control (Path) 2 | 38.2 | Control (Path) 4 | 31.6 |
| Temporal Ctx | | Parietal Ctx | |

TABLE BBC
Panel 1.3D

| | Rel. Exp. (%) Ag2900, | | Rel. Exp. (%) Ag2900, |
|---|---|---|---|
| Tissue Name | Run 159996755 | Tissue Name | Run 159996755 |
| Liver adenocarcinoma | 22.7 | Kidney (fetal) | 14.1 |
| Pancreas | 1.8 | Renal ca. 786-0 | 15.1 |
| Pancreatic ca. CAPAN 2 | 2.8 | Renal ca. A498 | 23.7 |
| Adrenal gland | 22.1 | Renal ca. RXF 393 | 2.7 |
| Thyroid | 3.6 | Renal ca. ACHN | 3.0 |
| Salivary gland | 2.9 | Renal ca. UO-31 | 6.7 |
| Pituitary gland | 5.8 | Renal ca. TK-10 | 3.6 |
| Brain (fetal) | 10.7 | Liver | 6.0 |
| Brain (whole) | 12.3 | Liver (fetal) | 12.5 |
| Brain (amygdala) | 8.4 | Liver ca. | 11.8 |
| | | (hepatoblast) HepG2 | |
| Brain (cerebellum) | 9.3 | Lung | 15.0 |
| Brain (hippocampus) | 61.6 | Lung (fetal) | 6.9 |
| Brain (substantia nigra) | 5.1 | Lung ca. (small cell) | 11.1 |
| | | LX-1 | |
| Brain (thalamus) | 10.4 | Lung ca. (small cell) | 12.3 |
| | | NCI-H69 | |
| Cerebral Cortex | 22.4 | Lung ca. (s.cell var.) | 30.4 |
| | | SHP-77 | |
| Spinal cord | 4.7 | Lung ca. (large | 28.3 |
| | | cell)NCI-H460 | |
| glio/astro U87-MG | 26.1 | Lung ca. (non-sm. | 15.7 |
| | | cell) A549 | |
| glio/astro U-118-MG | 94.0 | Lung ca. (non-s.cell) | 15.0 |
| | | NCI-H23 | |
| astrocytoma SW1783 | 16.5 | Lung ca. (non-s.cell) | 4.2 |
| | | HOP-62 | |
| neuro*; met SK-N-AS | 50.0 | Lung ca. (non-s.cl) | 9.3 |
| | | NCI-H522 | |
| astrocytoma SF-539 | 9.5 | Lung ca. (squam.) | 8.4 |
| | | SW 900 | |
| astrocytoma SNB-75 | 10.9 | Lung ca. (squam.) | 2.8 |
| | | NCI-H596 | |
| glioma SNB-19 | 10.2 | Mammary gland | 10.1 |
| glioma U251 | 3.3 | Breast ca.* (pl.ef) | 21.8 |
| | | MCF-7 | |
| glioma SF-295 | 2.9 | Breast ca.* (pl.ef) | 100.0 |
| | | MDA-MB-231 | |
| Heart (fetal) | 2.1 | Breast ca.* (pl.ef) | 5.3 |
| | | T47D | |
| Heart | 3.1 | Breast ca. BT-549 | 40.9 |
| Skeletal muscle (fetal) | 3.0 | Breast ca. MDA-N | 12.0 |
| Skeletal muscle | 2.1 | Ovary | 3.0 |
| Bone marrow | 8.1 | Ovarian ca. OVCAR-3 | 7.3 |
| Thymus | 4.0 | Ovarian ca. OVCAR-4 | 0.9 |
| Spleen | 5.3 | Ovarian ca. OVCAR-5 | 5.9 |
| Lymph node | 4.0 | Ovarian ca. OVCAR-8 | 8.3 |
| Colorectal | 7.6 | Ovarian ca. IGROV-1 | 2.1 |
| Stomach | 2.7 | Ovarian ca.* (ascites) | 11.3 |
| | | SK-OV-3 | |
| Small intestine | 5.7 | Uterus | 3.0 |
| Colon ca. SW480 | 11.0 | Placenta | 12.6 |
| Colon ca.* | 7.2 | Prostate | 4.3 |
| SW620(SW480 met) | | | |
| Colon ca. HT29 | 7.7 | Prostate ca.* (bone | 24.7 |
| | | met)PC-3 | |
| Colon ca. HCT-116 | 23.0 | Testis | 7.7 |
| Colon ca. CaCo-2 | 15.7 | Melanoma | 11.3 |
| | | Hs688(A).T | |
| Colon ca. | 12.8 | Melanoma* (met) | 5.3 |
| tissue (ODO3866) | | Hs688(B).T | |
| Colon ca. HCC-2998 | 33.4 | Melanoma UACC-62 | 4.2 |
| Gastric ca.* (liver met) | 21.9 | Melanoma M14 | 4.4 |
| NCI-N87 | | | |
| Bladder | 11.8 | Melanoma LOX | 28.7 |
| | | IMVI | |
| Trachea | 10.2 | Melanoma* (met) | 17.9 |
| | | SK-MEL-5 | |
| Kidney | 4.3 | Adipose | 13.1 |

TABLE BBD
Panel 2D

| | Rel. Exp. (%) | | Rel. Exp. (%) |
|---|---|---|---|
| | Ag2900, Run | | Ag2900, Run |
| Tissue Name | 159996787 | Tissue Name | 159996787 |
| Normal Colon | 71.2 | Kidney Margin | 3.0 |
| | | 8120608 | |
| CC Well to Mod Diff | 24.5 | Kidney Cancer | 2.0 |
| (ODO3866) | | 8120613 | |
| CC Margin (ODO3866) | 20.6 | Kidney Margin | 2.5 |
| | | 8120614 | |
| CC Gr.2 rectosigmoid | 47.3 | Kidney Cancer | 7.1 |
| (ODO3868) | | 9010320 | |
| CC Margin (ODO3868) | 6.0 | Kidney Margin | 5.0 |
| | | 9010321 | |
| CC Mod Diff (ODO3920) | 39.2 | Normal Uterus | 5.3 |
| CC Margin (ODO3920) | 21.0 | Uterus Cancer 064011 | 16.5 |
| CC Gr.2 ascend colon | 69.3 | Normal Thyroid | 8.2 |
| (ODO3921) | | | |
| CC Margin (ODO3921) | 16.0 | Thyroid Cancer | 15.0 |
| | | 064010 | |
| CC from Partial | 41.8 | Thyroid Cancer | 7.7 |
| Hepatectomy (ODO4309) | | A302152 | |
| Mets | | | |
| Liver Margin (ODO4309) | 40.6 | Thyroid Margin | 11.0 |
| | | A302153 | |
| Colon mets to lung | 14.3 | Normal Breast | 12.7 |
| (OD04451-01) | | | |
| Lung Margin (OD04451- | 15.0 | Breast Cancer | 21.3 |
| 02) | | (OD04566) | |
| Normal Prostate 6546-1 | 11.7 | Breast Cancer | 63.7 |
| | | (OD04590-01) | |
| Prostate Cancer | 40.1 | Breast Cancer Mets | 48.0 |
| (OD04410) | | (OD04590-03) | |
| Prostate Margin | 32.3 | Breast Cancer | 32.1 |
| (OD04410) | | Metastasis | |
| | | (OD04655-05) | |
| Prostate Cancer | 26.4 | Breast Cancer 064006 | 21.8 |
| (OD04720-01) | | | |
| Prostate Margin | 35.8 | Breast Cancer 1024 | 6.3 |
| (OD04720-02) | | | |
| Normal Lung 061010 | 46.7 | Breast Cancer | 39.0 |
| | | 9100266 | |
| Lung Met to Muscle | 34.6 | Breast Margin | 14.4 |
| (ODO4286) | | 9100265 | |
| Muscle Margin | 12.4 | Breast Cancer | 37.1 |
| (ODO4286) | | A209073 | |
| Lung Malignant Cancer | 18.8 | Breast Margin | 14.4 |
| (OD03126) | | A2090734 | |
| Lung Margin (OD03126) | 16.7 | Normal Liver | 14.1 |
| Lung Cancer (OD04404) | 20.9 | Liver Cancer 064003 | 20.2 |
| Lung Margin (OD04404) | 15.3 | Liver Cancer 1025 | 7.8 |
| Lung Cancer (OD04565) | 15.8 | Liver Cancer 1026 | 5.6 |
| Lung Margin (OD04565) | 10.3 | Liver Cancer 6004-T | 6.0 |
| Lung Cancer (OD04237- | 29.1 | Liver Tissue 6004-N | 6.6 |
| 01) | | | |
| Lung Margin (OD04237- | 29.5 | Liver Cancer 6005-T | 6.6 |
| 02) | | | |
| Ocular Mel Met to Liver | 17.0 | Liver Tissue 6005-N | 6.7 |
| (ODO4310) | | | |
| Liver Margin (ODO4310) | 23.8 | Normal Bladder | 54.7 |
| Melanoma Mets to Lung | 16.7 | Bladder Cancer 1023 | 7.6 |
| (OD04321) | | | |
| Lung Margin (OD04321) | 20.6 | Bladder Cancer | 23.2 |
| | | A302173 | |
| Normal Kidney | 16.6 | Bladder Cancer | 100.0 |
| | | (OD04718-01) | |
| Kidney Ca, Nuclear grade | 15.8 | Bladder Normal | 30.4 |
| 2 (OD04338) | | Adjacent (OD04718- | |
| | | 03) | |
| Kidney Margin | 11.5 | Normal Ovary | 2.3 |
| (OD04338) | | | |
| Kidney Ca Nuclear grade | 10.2 | Ovarian Cancer | 30.6 |
| 1/2 (OD04339) | | 064008 | |
| Kidney Margin | 15.9 | Ovarian Cancer | 37.6 |
| (OD04339) | | (OD04768-07) | |
| Kidney Ca, Clear cell | 28.7 | Ovary Margin | 14.8 |
| type (OD04340) | | (OD04768-08) | |
| Kidney Margin | 15.5 | Normal Stomach | 17.9 |
| (OD04340) | | | |
| Kidney Ca, Nuclear grade | 12.3 | Gastric Cancer | 10.0 |
| 3 (OD04348) | | 9060358 | |
| Kidney Margin | 16.0 | Stomach Margin | 23.0 |
| (OD04348) | | 9060359 | |
| Kidney Cancer | 12.5 | Gastric Cancer | 35.8 |
| (OD04622-01) | | 9060395 | |
| Kidney Margin | 3.6 | Stomach Margin | 27.5 |
| (OD04622-03) | | 9060394 | |
| Kidney Cancer | 9.5 | Gastric Cancer | 66.0 |
| (OD04450-01) | | 9060397 | |
| Kidney Margin | 13.0 | Stomach Margin 9060396 | 18.0 |
| (OD04450-03) | | | |
| Kidney Cancer 8120607 | 3.1 | Gastric Cancer | 62.0 |
| | | 064005 | |

TABLE BBE
Panel 4D

| | Rel. Exp. (%) | | Rel. Exp. (%) |
|---|---|---|---|
| | Ag2900, Run | | Ag2900, Run |
| Tissue Name | 159996820 | Tissue Name | 159996820 |
| Secondary Th1 act | 68.3 | HUVEC IL-1beta | 10.5 |
| Secondary Th2 act | 67.8 | HUVEC IFN gamma | 15.5 |
| Secondary Tr1 act | 73.2 | HUVEC TNF alpha + IFN | 16.0 |
| | | gamma | |
| Secondary Th1 rest | 8.2 | HUVEC TNF alpha + IL4 | 18.8 |
| Secondary Th2 rest | 18.6 | HUVEC IL-11 | 13.1 |
| Secondary Tr1 rest | 10.1 | Lung Microvascular EC | 18.8 |
| | | none | |
| Primary Th1 act | 75.8 | Lung Microvascular EC | 25.2 |
| | | TNF alpha + IL-1beta | |
| Primary Th2 act | 60.7 | Microvascular Dermal EC | 30.8 |
| | | none | |
| Primary Tr1 act | 74.7 | Microsvasular Dermal EC | 27.9 |
| | | TNF alpha + IL-1beta | |
| Primary Th1 rest | 42.0 | Bronchial epithelium | 16.8 |
| | | TNF alpha + IL1beta | |
| Primary Th2 rest | 29.3 | Small airway epithelium | 11.7 |
| | | none | |
| Primary Tr1 rest | 16.3 | Small airway epithelium | 79.0 |
| | | TNF alpha + IL-1beta | |
| CD45RA CD4 | 34.4 | Coronery artery SMC rest | 19.2 |
| lymphocyte act | | | |
| CD45RO CD4 | 53.6 | Coronery artery SMC | 7.6 |
| lymphocyte act | | TNF alpha + IL-1beta | |
| CD8 lymphocyte act | 35.8 | Astrocytes rest | 8.6 |
| Secondary CD8 | 39.0 | Astrocytes TNF alpha + | 7.2 |
| lymphocyte rest | | IL-1beta | |
| Secondary CD8 | 33.9 | KU-812 (Basophil) rest | 7.8 |
| lymphocyte act | | | |
| CD4 lymphocyte none | 6.2 | KU-812 (Basophil) | 40.6 |
| | | PMA/ionomycin | |
| 2ry Th1/Th2/Tr1_anti- | 11.7 | CCD1106 (Keratinocytes) | 30.4 |
| CD95 CH11 | | none | |
| LAK cells rest | 25.2 | CCD1106 (Keratinocytes) | 13.6 |
| | | TNF alpha + IL-1beta | |
| LAK cells IL-2 | 23.8 | Liver cirrhosis | 3.9 |
| LAK cells IL-2 + IL-12 | 22.5 | Lupus kidney | 1.3 |
| LAK cells IL-2 + IFN | 36.3 | NCI-H292 none | 87.1 |
| gamma | | | |
| LAK cells IL-2 + IL-18 | 29.7 | NCI-H292 IL-4 | 91.4 |
| LAK cells | 47.0 | NCI-H292 IL-9 | 92.0 |
| PMA/ionomycin | | | |
| NK Cells IL-2 rest | 13.7 | NCI-H292 IL-13 | 39.8 |
| Two Way MLR 3 day | 15.7 | NCI-H292 IFN gamma | 47.0 |
| Two Way MLR 5 day | 15.6 | HPAEC none | 18.0 |
| Two Way MLR 7 day | 12.6 | HPAEC TNF alpha + IL- | 26.1 |
| | | 1beta | |
| PBMC rest | 7.5 | Lung fibroblast none | 22.2 |
| PBMC PWM | 100.0 | Lung fibroblast TNF alpha + | 16.8 |
| | | IL-1beta | |
| PBMC PHA-L | 42.6 | Lung fibroblast IL-4 | 62.4 |
| Ramos (B cell) none | 17.0 | Lung fibroblast IL-9 | 39.5 |
| Ramos (B cell) | 54.0 | Lung fibroblast IL-13 | 35.4 |
| ionomycin | | | |
| B lymphocytes PWM | 73.7 | Lung fibroblast IFN | 70.2 |
| | | gamma | |
| B lymphocytes CD40L | 12.9 | Dermal fibroblast | 49.3 |
| and IL-4 | | CCD1070 rest | |
| EOL-1 dbcAMP | 9.5 | Dermal fibroblast | 92.7 |
| | | CCD1070 TNF alpha | |
| EOL-1 dbcAMP | 22.8 | Dermal fibroblast | 29.5 |
| PMA/ionomycin | | CCD1070 IL-1beta | |
| Dendritic cells none | 13.7 | Dermal fibroblast IFN | 27.7 |
| | | gamma | |
| Dendritic cells LPS | 15.0 | Dermal fibroblast IL-4 | 41.5 |
| Dendritic cells anti- | 19.1 | IBD Colitis 2 | 1.1 |
| CD40 | | | |
| Monocytes rest | 10.7 | IBD Crohn's | 2.6 |
| Monocytes LPS | 4.3 | Colon | 14.8 |
| Macrophages rest | 26.1 | Lung | 21.5 |
| Macrophages LPS | 15.3 | Thymus | 25.0 |
| HUVEC none | 30.8 | Kidney | 37.4 |
| HUVEC starved | 34.6 | | |

CNS_neurodegeneration_v1.0 Summary: Ag2900 This panel confirms the expression of this gene at low levels in the brain in an independent group of individuals. However, no differential expression of this gene was detected between Alzheimer's diseased postmortem brains and those of non-demented controls in this experiment. Please see Panel 1.3D for a discussion of the potential utility of this gene in treatment of central nervous system disorders.

Panel 1.3D Summary: Ag2900 The CG56633-01 gene is expressed at moderate levels in the cancer cell lines in this panel, with highest expression in a breast cancer cell line (CT=27). Expression of this gene could potentially be used as a diagnostic marker of cell proliferation and hence as a diagnostic marker for cancer.

This gene also has moderate levels of expression in adipose, liver, heart, skeletal muscle, adrenal, pituitary, thyroid and pancreas. Therefore, therapeutic modulation of this gene product may be a treatment for endocrine and metabolic diseases, including obesity and Types 1 and 2 diabetes.

Overall, this gene, a translation initiation factor homolog, exhibits brain-preferential expression, particularly in the hippocampus, a structure critical for learning and memory. The processes of learning and memory are subject to regulation by mechanisms of translational and transcriptional control, including the regulation elongation factor phosphorylation by the memory-mediating NMDA receptor. The hippocampus-preferential expression of this gene suggests that it plays a role in translationally-mediated learning and memory processes. Therefore, agents that modulate the activity and function of this gene product may have utility in treating CNS disorders involving memory deficits, including Alzheimer's disease and aging.

References:

Scheetz A J, Nairn A C, Constantine-Paton M. N-methyl-D-aspartate receptor activation and visual activity induce elongation factor-2 phosphorylation in amphibian tecta: a role for N-methyl-D-aspartate receptors in controlling protein synthesis. Proc Natl Acad Sci USA Dec. 23, 1997;94(26):14770-5

N-methyl-D-aspartate receptor (NMDAR) activation has been implicated in forms of synaptic plasticity involving long-term changes in neuronal structure, function, or protein expression. Transcriptional alterations have been correlated with NMDAR-mediated synaptic plasticity, but the problem of rapidly targeting new proteins to particular synapses is unsolved. One potential solution is synapse-specific protein translation, which is suggested by dendritic localization of numerous transcripts and subsynaptic polyribosomes. We report here a mechanism by which NMDAR activation at synapses may control this protein synthetic machinery. In intact tadpole tecta, NMDAR activation leads to phosphorylation of a subset of proteins, one of which we now identify as the eukaryotic translation elongation factor 2 (eEF2). Phosphorylation of eEF2 halts protein synthesis and may prepare cells to translate a new set of mRNAs. We show that NMDAR activation-induced eEF2 phosphorylation is widespread in tadpole tecta. In contrast, in adult tecta, where synaptic plasticity is reduced, this phosphorylation is restricted to short dendritic regions that process binocular information. Biochemical and anatomical evidence shows that this NMDAR activation-induced eEF2 phosphorylation is localized to subsynaptic sites. Moreover, eEF2 phosphorylation is induced by visual stimulation, and NMDAR blockade before stimulation eliminates this effect. Thus, NMDAR activation, which is known to mediate synaptic changes in the developing frog, could produce local postsynaptic alterations in protein synthesis by inducing eEF2 phosphorylation.

Panel 2D Summary: Ag2900 The CG56633-01 gene is expressed at increased levels in colon, breast and bladder cancers compared to the normal adjacent tissue samples. Therefore, expression of this gene could be of use as a marker for these cancers.

Panel 4D Summary: Ag2900 The CG56633-01 gene is expressed in a number of preparations of activated T lymphocytes at levels greater than in resting T cells. Therefore, small molecule antagonists of the CG56633-01 gene product may reduce T cell activation and thus reduce or eliminate the symptoms in patients with autoimmune and inflammatory diseases, such as Crohn's disease, ulcerative colitis, multiple sclerosis, chronic obstructive pulmonary disease, asthma, emphysema, rheumatoid arthritis, lupus erythematosus, or psoriasis.

NOV60a and NOV60b

Expression of gene NOV60a and variant NOV60b was assessed using the primer-probe sets Ag041b and Ag41, described in Tables BCA and BCB. Results of the RTQ-PCR runs are shown in Tables BCC, BCD, BCE, BCF, BCG, BCH and BCI.

TABLE BCA
Probe Name Ag041b

| | | | Start | |
|---|---|---|---|---|
| Primers | Sequences | Length | Position | SEQ ID NO: |
| Forward | 5′-gtagtaggtgcgcgtggtcat-3′ | 21 | 486 | 1231 |
| Probe | TET-5′-accatagccgggcagcgcatg-3′-TAMRA | 21 | 455 | 1232 |
| Reverse | 5′-caacggagacaactgcttcaac-3′ | 22 | 431 | 1233 |

TABLE BCB
Probe Name Ag41

| | | | Start | |
|---|---|---|---|---|
| Primers | Sequences | Length | Position | SEQ ID NO: |
| Forward | 5′-gtaggtgcgcgtggtcatg-3′ | 19 | 485 | 1234 |
| Probe | TET-5′-ccatgcgctgcccggctatg-3′-TAMRA | 20 | 454 | 1235 |
| Reverse | 5′-cctacaacggagacaactgcttc-3′ | 23 | 427 | 1236 |

TABLE BCC
CNS_neurodegeneration_v1.0

| | Rel. Exp. (%) Ag041b, | | Rel. Exp. (%) Ag041b, |
|---|---|---|---|
| Tissue Name | Run 206231412 | Tissue Name | Run 206231412 |
| AD 1 Hippo | 20.7 | Control (Path) 3 | 9.7 |
| | | Temporal Ctx | |
| AD 2 Hippo | 44.1 | Control (Path) 4 | 70.2 |
| | | Temporal Ctx | |
| AD 3 Hippo | 9.5 | AD 1 Occipital Ctx | 17.9 |
| AD 4 Hippo | 17.4 | AD 2 Occipital Ctx | 0.0 |
| | | (Missing) | |
| AD 5 Hippo | 97.9 | AD 3 Occipital Ctx | 5.8 |
| AD 6 Hippo | 65.1 | AD 4 Occipital Ctx | 39.8 |
| Control 2 Hippo | 66.0 | AD 5 Occipital Ctx | 76.8 |
| Control 4 Hippo | 18.4 | AD 6 Occipital Ctx | 20.0 |
| Control (Path) 3 | 11.3 | Control 1 Occipital | 5.3 |
| Hippo | | Ctx | |
| AD 1 Temporal Ctx | 11.3 | Control 2 Occipital | 82.4 |
| | | Ctx | |
| AD 2 Temporal Ctx | 54.3 | Control 3 Occipital | 36.9 |
| | | Ctx | |
| AD 3 Temporal Ctx | 9.5 | Control 4 Occipital | 12.1 |
| | | Ctx | |
| AD 4 Temporal Ctx | 39.8 | Control (Path) 1 | 95.3 |
| | | Occipital Ctx | |
| AD 5 Inf Temporal | 80.7 | Control (Path) 2 | 33.4 |
| Ctx | | Occipital Ctx | |
| AD 5 Sup Temporal | 57.8 | Control (Path) 3 | 4.4 |
| Ctx | | Occipital Ctx | |
| AD 6 Inf Temporal | 52.1 | Control (Path) 4 | 53.6 |
| Ctx | | Occipital Ctx | |
| AD 6 Sup Temporal | 54.3 | Control 1 Parietal | 9.3 |
| Ctx | | Ctx | |
| Control 1 Temporal | 13.8 | Control 2 Parietal | 49.0 |
| Ctx | | Ctx | |
| Control 2 Temporal | 66.0 | Control 3 Parietal | 34.6 |
| Ctx | | Ctx | |
| Control 3 Temporal | 36.3 | Control (Path) 1 | 100.0 |
| Ctx | | Parietal Ctx | |
| Control 3 Temporal | 19.8 | Control (Path) 2 | 44.4 |
| Ctx | | Parietal Ctx | |
| Control (Path) 1 | 85.3 | Control (Path) 3 | 6.9 |
| Temporal Ctx | | Parietal Ctx | |
| Control (Path) 2 | 73.7 | Control (Path) 4 | 72.2 |
| Temporal Ctx | | Parietal Ctx | |

TABLE BCD
Panel 1

| | Rel. Exp. (%) | Rel. Exp. (%) | | Rel. Exp. (%) | Rel. Exp. (%) |
|---|---|---|---|---|---|
| | Ag41, Run | Ag41, Run | | Ag41, Run | Ag41, Run |
| Tissue Name | 97804013 | 97807227 | Tissue Name | 97804013 | 97807227 |
| Endothelial cells | 0.0 | 0.2 | Renal ca. 786-0 | 0.0 | 0.4 |
| Endothelial cells | 0.0 | 0.3 | Renal ca. A498 | 11.6 | 32.8 |
| (treated) | | | | | |
| Pancreas | 0.1 | 4.2 | Renal ca. RXF | 3.6 | 1.0 |
| | | | 393 | | |
| Pancreatic ca. | 0.0 | 0.5 | Renal ca. | 0.0 | 1.1 |
| CAPAN 2 | | | ACHN | | |
| Adrenal gland | 1.2 | 15.3 | Renal ca. UO- | 0.0 | 0.9 |
| | | | 31 | | |
| Thyroid | 0.0 | 4.4 | Renal ca. TK- | 0.0 | 0.2 |
| | | | 10 | | |
| Salivary gland | 0.1 | 6.5 | Liver | 0.9 | 8.3 |
| Pituitary gland | 0.2 | 6.0 | Liver (fetal) | 0.1 | 3.3 |
| Brain (fetal) | 0.0 | 3.7 | Liver ca. | 0.2 | 5.6 |
| | | | (hepatoblast) | | |
| | | | HepG2 | | |
| Brain (whole) | 0.0 | 35.4 | Lung | 0.0 | 1.8 |
| Brain (amygdala) | 6.4 | 60.7 | Lung (fetal) | 0.0 | 1.1 |
| Brain | 4.2 | 25.5 | Lung ca. (small | 0.0 | 1.0 |
| (cerebellum) | | | cell) LX-1 | | |
| Brain | 6.1 | 54.0 | Lung ca. (small | 0.0 | 0.7 |
| (hippocampus) | | | cell) NCI-H69 | | |
| Brain (substantia | 3.4 | 35.6 | Lung ca. (s.cell | 0.0 | 0.2 |
| nigra) | | | var.) SHP-77 | | |
| Brain (thalamus) | 11.1 | 100.0 | Lung ca. (large | 15.9 | 2.7 |
| | | | cell)NCI-H460 | | |
| Brain | 0.2 | 6.3 | Lung ca. (non- | 0.0 | 1.4 |
| (hypothalamus) | | | sm. cell) A549 | | |
| Spinal cord | 4.1 | 14.0 | Lung ca. (non- | 5.7 | 14.7 |
| | | | s.cell) NCI-H23 | | |
| glio/astro U87- | 0.0 | 0.0 | Lung ca. (non- | 0.0 | 0.4 |
| MG | | | s.cell) HOP-62 | | |
| glio/astro U-118- | 0.0 | 0.2 | Lung ca. (non- | 0.2 | 6.3 |
| MG | | | s.cl) NCI-H522 | | |
| astrocytoma | 0.0 | 0.4 | Lung ca. | 0.3 | 6.9 |
| SW1783 | | | (squam.) SW | | |
| | | | 900 | | |
| neuro*; met SK- | 0.0 | 0.3 | Lung ca. | 0.0 | 0.4 |
| N-AS | | | (squam.) NCI- | | |
| | | | H596 | | |
| astrocytoma SF- | 0.0 | 0.8 | Mammary | 100.0 | 10.5 |
| 539 | | | gland | | |
| astrocytoma SNB- | 0.3 | 4.7 | Breast ca.* | 0.0 | 0.0 |
| 75 | | | (pl.ef) MCF-7 | | |
| glioma SNB-19 | 0.3 | 2.3 | Breast ca.* | 0.0 | 0.2 |
| | | | (pl.ef) MDA- | | |
| | | | MB-231 | | |
| glioma U251 | 7.6 | 0.5 | Breast ca.* (pl. | 0.8 | 7.4 |
| | | | ef) T47D | | |
| glioma SF-295 | 0.0 | 0.8 | Breast ca. BT- | 0.0 | 0.1 |
| | | | 549 | | |
| Heart | 6.8 | 49.0 | Breast ca. | 0.0 | 0.0 |
| | | | MDA-N | | |
| Skeletal muscle | 0.5 | 6.0 | Ovary | 0.9 | 13.0 |
| Bone marrow | 0.0 | 1.0 | Ovarian ca. | 0.0 | 1.0 |
| | | | OVCAR-3 | | |
| Thymus | 0.0 | 0.9 | Ovarian ca. | 0.0 | 0.4 |
| | | | OVCAR-4 | | |
| Spleen | 0.0 | 2.5 | Ovarian ca. | 0.4 | 7.1 |
| | | | OVCAR-5 | | |
| Lymph node | 0.0 | 1.1 | Ovarian ca. | 0.0 | 0.2 |
| | | | OVCAR-8 | | |
| Colon (ascending) | 0.0 | 1.7 | Ovarian ca. | 0.0 | 0.5 |
| | | | IGROV-1 | | |
| Stomach | 4.6 | 3.2 | Ovarian ca. | 3.8 | 0.7 |
| | | | (ascites) SK- | | |
| | | | OV-3 | | |
| Small intestine | 0.1 | 3.4 | Uterus | 0.7 | 11.7 |
| Colon ca. SW480 | 0.0 | 0.3 | Placenta | 0.9 | 9.9 |
| Colon ca.* | 0.0 | 0.6 | Prostate | 0.3 | 7.7 |
| SW620 (SW480 | | | | | |
| met) | | | | | |
| Colon ca. HT29 | 0.0 | 0.0 | Prostate ca.* | 0.0 | 0.1 |
| | | | (bone met) PC-3 | | |
| Colon ca. HCT- | 0.0 | 0.8 | Testis | 0.1 | 3.0 |
| 116 | | | | | |
| Colon ca. CaCo-2 | 0.0 | 0.5 | Melanoma | 0.0 | 0.9 |
| | | | Hs688(A).T | | |
| Colon ca. HCT-15 | 0.0 | 0.4 | Melanoma* | 0.0 | 2.3 |
| | | | (met) | | |
| | | | Hs688(B).T | | |
| Colon ca. HCC- | 0.0 | 0.0 | Melanoma | 0.0 | 0.3 |
| 2998 | | | UACC-62 | | |
| Gastric ca.* (liver | 0.0 | 0.3 | Melanoma M14 | 0.0 | 0.1 |
| met) NCI-N87 | | | | | |
| Bladder | 0.0 | 2.5 | Melanoma | 0.0 | 0.0 |
| | | | LOX IMVI | | |
| Trachea | 0.0 | 2.0 | Melanoma* | 0.0 | 0.1 |
| | | | (met) SK-MEL-5 | | |
| Kidney | 1.0 | 11.6 | Melanoma SK- | 0.1 | 1.5 |
| | | | MEL-28 | | |
| Kidney (fetal) | 0.0 | 3.4 | | | |

TABLE BCE
Panel 1.1

| | Rel. Exp. (%) Ag041b, | | Rel. Exp. (%) Ag041b, |
|---|---|---|---|
| Tissue Name | Run 109666937 | Tissue Name | Run 109666937 |
| Adrenal gland | 11.5 | Renal ca. UO-31 | 1.3 |
| Bladder | 2.4 | Renal ca. RXF 393 | 0.4 |
| Brain (amygdala) | 25.2 | Liver | 9.9 |
| Brain (cerebellum) | 57.4 | Liver (fetal) | 2.9 |
| Brain (hippocampus) | 77.4 | Liver ca. | 2.7 |
| | | (hepatoblast) HepG2 | |
| Brain (substantia | 57.4 | Lung | 1.6 |
| nigra) | | | |
| Brain (thalamus) | 47.6 | Lung (fetal) | 3.3 |
| Cerebral Cortex | 100.0 | Lung ca. (non-s.cell) | 0.3 |
| | | HOP-62 | |
| Brain (fetal) | 5.5 | Lung ca. (large | 1.4 |
| | | cell)NCI-H460 | |
| Brain (whole) | 71.2 | Lung ca. (non-s.cell) | 6.2 |
| | | NCI-H23 | |
| glio/astro U-118-MG | 0.2 | Lung ca. (non-s.cl) | 9.2 |
| | | NCI-H522 | |
| astrocytoma SF-539 | 1.1 | Lung ca. (non-sm. | 1.7 |
| | | cell) A549 | |
| astrocytoma SNB-75 | 5.7 | Lung ca. (s.cell var.) | 0.2 |
| | | SHP-77 | |
| astrocytoma SW1783 | 0.6 | Lung ca. (small cell) | 1.4 |
| | | LX-1 | |
| glioma U251 | 0.2 | Lung ca. (small cell) | 0.6 |
| | | NCI-H69 | |
| glioma SF-295 | 1.2 | Lung ca. (squam.) | 5.9 |
| | | SW 900 | |
| glioma SNB-19 | 2.8 | Lung ca. (squam.) | 0.5 |
| | | NCI-H596 | |
| glio/astro U87-MG | 0.0 | Lymph node | 1.4 |
| neuro*; met SK-N-AS | 0.2 | Spleen | 2.5 |
| Mammary gland | 6.3 | Thymus | 0.5 |
| Breast ca. BT-549 | 0.0 | Ovary | 6.7 |
| Breast ca. MDA-N | 0.0 | Ovarian ca. IGROV-1 | 0.5 |
| Breast ca.* (pl.ef) | 8.0 | Ovarian ca. | 1.1 |
| T47D | | OVCAR-3 | |
| Breast ca.* (pl.ef) | 0.0 | Ovarian ca. | 0.2 |
| MCF-7 | | OVCAR-4 | |
| Breast ca.* (pl.ef) | 0.3 | Ovarian ca. | 9.8 |
| MDA-MB-231 | | OVCAR-5 | |
| Small intestine | 6.7 | Ovarian ca. | 0.0 |
| | | OVCAR-8 | |
| Colorectal | 0.5 | Ovarian ca.* | 0.7 |
| | | (ascites) SK-OV-3 | |
| Colon ca. HT29 | 0.0 | Pancreas | 11.5 |
| Colon ca. CaCo-2 | 0.9 | Pancreatic ca. | 0.4 |
| | | CAPAN 2 | |
| Colon ca. HCT-15 | 0.1 | Pituitary gland | 6.6 |
| Colon ca. HCT-116 | 0.4 | Placenta | 9.5 |
| Colon ca. HCC-2998 | 0.0 | Prostate | 7.0 |
| Colon ca. SW480 | 0.1 | Prostate ca.* (bone | 0.0 |
| | | met) PC-3 | |
| Colon ca.* SW620 | 1.3 | Salivary gland | 6.2 |
| (SW480 met) | | | |
| Stomach | 8.0 | Trachea | 2.3 |
| Gastric ca. (liver met) | 0.3 | Spinal cord | 14.3 |
| NCI-N87 | | | |
| Heart | 92.7 | Testis | 3.1 |
| Skeletal muscle (Fetal) | 4.9 | Thyroid | 6.1 |
| Skeletal muscle | 16.8 | Uterus | 4.7 |
| Endothelial cells | 1.0 | Melanoma M14 | 0.0 |
| Heart (Fetal) | 33.9 | Melanoma LOX | 0.0 |
| | | IMVI | |
| Kidney | 16.8 | Melanoma UACC- | 0.1 |
| | | 62 | |
| Kidney (fetal) | 3.1 | Melanoma SK-MEL- | 0.7 |
| | | 28 | |
| Renal ca. 786-0 | 0.2 | Melanoma* (met) | 0.0 |
| | | SK-MEL-5 | |
| Renal ca. A498 | 30.8 | Melanoma | 1.1 |
| | | Hs688(A).T | |
| Renal ca. ACHN | 1.2 | Melanoma* (met) | 1.5 |
| | | Hs688(B).T | |
| Renal ca. TK-10 | 0.2 | | |

TABLE BCF
Panel 1.3D

| | Rel. Exp. (%) | | Rel. |
|---|---|---|---|
| | Ag041b, Run | | Exp. (%) Ag041b, |
| Tissue Name | 150010102 | Tissue Name | Run 150010102 |
| Liver | 0.0 | Kidney (fetal) | 0.5 |
| adenocarcinoma | | | |
| Pancreas | 0.6 | Renal ca. 786-0 | 0.0 |
| Pancreatic ca. | 0.1 | Renal ca. A498 | 11.0 |
| CAPAN 2 | | | |
| Adrenal gland | 2.0 | Renal ca. RXF | 0.1 |
| | | 393 | |
| Thyroid | 1.0 | Renal ca. ACHN | 0.2 |
| Salivary gland | 0.6 | Renal ca. UO-31 | 0.3 |
| Pituitary gland | 0.8 | Renal ca. TK-10 | 0.0 |
| Brain (fetal) | 0.5 | Liver | 1.0 |
| Brain (whole) | 25.0 | Liver (fetal) | 0.8 |
| Brain (amygdala) | 25.3 | Liver ca. | 0.6 |
| | | (hepatoblast) HepG2 | |
| Brain | 2.7 | Lung | 0.6 |
| (cerebellum) | | | |
| Brain | 100.0 | Lung (fetal) | 0.9 |
| (hippocampus) | | | |
| Brain (substantia | 3.5 | Lung ca. (small | 0.1 |
| nigra) | | cell) LX-1 | |
| Brain (thalamus) | 20.7 | Lung ca. (small | 0.2 |
| | | cell) NCI-H69 | |
| Cerebral Cortex | 63.3 | Lung ca. (s.cell | 0.1 |
| | | var.) SHP-77 | |
| Spinal cord | 3.6 | Lung ca. (large | 0.1 |
| | | cell)NCI-H460 | |
| glio/astro U87- | 0.0 | Lung ca. (non- | 0.2 |
| MG | | sm. cell) A549 | |
| glio/astro U-118- | 0.1 | Lung ca. (non- | 3.4 |
| MG | | s.cell) NCI-H23 | |
| astrocytoma | 0.1 | Lung ca. (non- | 0.1 |
| SW1783 | | s.cell) HOP-62 | |
| neuro*; met SK- | 0.0 | Lung ca. (non- | 1.2 |
| N-AS | | s.cl) NCI-H522 | |
| astrocytoma SF- | 0.1 | Lung ca. | 0.5 |
| 539 | | (squam.) SW 900 | |
| astrocytoma | 0.8 | Lung ca. | 0.0 |
| SNB-75 | | (squam.) NCI-H596 | |
| glioma SNB-19 | 0.4 | Mammary gland | 1.2 |
| glioma U251 | 0.1 | Breast ca.* | 0.0 |
| | | (pl.ef) MCF-7 | |
| glioma SF-295 | 0.4 | Breast ca.* | 0.1 |
| | | (pl.ef) MDA-MB-231 | |
| Heart (fetal) | 21.9 | Breast ca.* | 1.0 |
| | | (pl.ef) T47D | |
| Heart | 3.7 | Breast ca. BT- | 0.0 |
| | | 549 | |
| Skeletal muscle | 10.7 | Breast ca. MDA-N | 0.0 |
| (fetal) | | | |
| Skeletal muscle | 0.2 | Ovary | 8.6 |
| Bone marrow | 0.2 | Ovarian ca. | 0.1 |
| | | OVCAR-3 | |
| Thymus | 0.1 | Ovarian ca. | 0.1 |
| | | OVCAR-4 | |
| Spleen | 1.7 | Ovarian ca. | 0.8 |
| | | OVCAR-5 | |
| Lymph node | 0.4 | Ovarian ca. | 0.0 |
| | | OVCAR-8 | |
| Colorectal | 1.7 | Ovarian ca. | 0.1 |
| | | IGROV-1 | |
| Stomach | 1.5 | Ovarian ca.* | 0.1 |
| | | (ascites) SK-OV-3 | |
| Small intestine | 1.6 | Uterus | 1.9 |
| Colon ca. | 0.2 | Placenta | 1.7 |
| SW480 | | | |
| Colon ca.* | 0.2 | Prostate | 0.9 |
| SW620(SW480 met) | | | |
| Colon ca. HT29 | 0.0 | Prostate ca.* | 0.0 |
| | | (bone met)PC-3 | |
| Colon ca. HCT- | 0.1 | Testis | 0.8 |
| 116 | | | |
| Colon ca. CaCo-2 | 0.1 | Melanoma Hs688(A).T | 1.3 |
| Colon ca. | 0.3 | Melanoma* | 0.7 |
| tissue(ODO3866) | | (met) Hs688(B).T | |
| Colon ca. HCC- | 0.0 | Melanoma | 0.0 |
| 2998 | | UACC-62 | |
| Gastric ca.* | 0.0 | Melanoma M14 | 0.0 |
| (liver met) NCI-N87 | | | |
| Bladder | 0.6 | Melanoma LOX IMVI | 0.0 |
| Trachea | 0.7 | Melanoma* | 0.0 |
| | | (met) SK-MEL-5 | |
| Kidney | 0.7 | Adipose | 1.6 |

TABLE BCG
Panel 2D

| | Rel. Exp. (%) | Rel. Exp. (%) | | Rel. Exp. (%) | Rel. Exp. (%) |
|---|---|---|---|---|---|
| | Ag041b, Run | Ag41, Run | | Ag041b, Run | Ag41, Run |
| Tissue Name | 157096248 | 157938256 | Tissue Name | 157096248 | 157938256 |
| Normal Colon | 15.7 | 21.5 | Kidney | 9.2 | 12.9 |
| | | | Margin | | |
| | | | 8120608 | | |
| CC Well to Mod | 4.4 | 2.9 | Kidney Cancer | 4.9 | 7.2 |
| Diff (ODO3866) | | | 8120613 | | |
| CC Margin | 6.3 | 7.3 | Kidney | 14.4 | 16.6 |
| (ODO3866) | | | Margin | | |
| | | | 8120614 | | |
| CC Gr.2 | 3.3 | 2.3 | Kidney Cancer | 63.7 | 69.3 |
| rectosigmoid | | | 9010320 | | |
| (ODO3868) | | | | | |
| CC Margin | 8.2 | 8.6 | Kidney | 33.0 | 42.3 |
| (ODO3868) | | | Margin | | |
| | | | 9010321 | | |
| CC Mod Diff | 4.9 | 6.9 | Normal Uterus | 8.5 | 14.9 |
| (ODO3920) | | | | | |
| CC Margin | 8.8 | 11.7 | Uterus Cancer | 16.2 | 14.3 |
| (ODO3920) | | | 064011 | | |
| CC Gr.2 ascend | 2.4 | 1.9 | Normal | 5.7 | 8.4 |
| colon | | | Thyroid | | |
| (ODO3921) | | | | | |
| CC Margin | 3.4 | 4.1 | Thyroid | 8.4 | 12.1 |
| (ODO3921) | | | Cancer 064010 | | |
| CC from Partial | 2.1 | 7.1 | Thyroid | 8.5 | 9.9 |
| Hepatectomy | | | Cancer | | |
| (ODO4309) Mets | | | A302152 | | |
| Liver Margin | 15.1 | 24.3 | Thyroid | 9.2 | 12.6 |
| (ODO4309) | | | Margin | | |
| | | | A302153 | | |
| Colon mets to | 0.8 | 1.2 | Normal Breast | 24.3 | 42.3 |
| lung (OD04451- | | | | | |
| 01) | | | | | |
| Lung Margin | 2.3 | 3.1 | Breast Cancer | 1.8 | 2.1 |
| (OD04451-02) | | | (OD04566) | | |
| Normal Prostate | 9.7 | 9.0 | Breast Cancer | 5.2 | 3.9 |
| 6546-1 | | | (OD04590-01) | | |
| Prostate Cancer | 13.6 | 12.4 | Breast Cancer | 8.5 | 9.9 |
| (OD04410) | | | Mets | | |
| | | | (OD04590-03) | | |
| Prostate Margin | 18.3 | 19.3 | Breast Cancer | 8.8 | 11.7 |
| (OD04410) | | | Metastasis | | |
| | | | (OD04655-05) | | |
| Prostate Cancer | 19.6 | 28.1 | Breast Cancer | 10.3 | 12.9 |
| (OD04720-01) | | | 064006 | | |
| Prostate Margin | 33.4 | 36.9 | Breast Cancer | 15.4 | 16.2 |
| (OD04720-02) | | | 1024 | | |
| Normal Lung | 7.2 | 10.9 | Breast Cancer | 3.8 | 5.7 |
| 061010 | | | 9100266 | | |
| Lung Met to | 0.7 | 1.2 | Breast Margin | 4.0 | 6.3 |
| Muscle | | | 9100265 | | |
| (ODO4286) | | | | | |
| Muscle Margin | 7.9 | 9.7 | Breast Cancer | 4.4 | 7.2 |
| (ODO4286) | | | A209073 | | |
| Lung Malignant | 3.0 | 3.7 | Breast Margin | 8.2 | 8.4 |
| Cancer | | | A2090734 | | |
| (OD03126) | | | | | |
| Lung Margin | 8.2 | 12.9 | Normal Liver | 21.2 | 20.3 |
| (OD03126) | | | | | |
| Lung Cancer | 6.3 | 9.9 | Liver Cancer | 1.8 | 1.4 |
| (OD04404) | | | 064003 | | |
| Lung Margin | 14.8 | 15.5 | Liver Cancer | 25.5 | 35.8 |
| (OD04404) | | | 1025 | | |
| Lung Cancer | 12.0 | 21.6 | Liver Cancer | 23.3 | 25.5 |
| (OD04565) | | | 1026 | | |
| Lung Margin | 7.5 | 5.7 | Liver Cancer | 64.6 | 60.3 |
| (OD04565) | | | 6004-T | | |
| Lung Cancer | 3.9 | 4.5 | Liver Tissue | 19.8 | 23.0 |
| (OD04237-01) | | | 6004-N | | |
| Lung Margin | 6.0 | 8.9 | Liver Cancer | 33.2 | 35.8 |
| (OD04237-02) | | | 6005-T | | |
| Ocular Mel Met | 4.0 | 4.9 | Liver Tissue | 17.4 | 22.8 |
| to Liver | | | 6005-N | | |
| (ODO4310) | | | | | |
| Liver Margin | 11.7 | 17.7 | Normal | 6.3 | 5.3 |
| (ODO4310) | | | Bladder | | |
| Melanoma Mets | 0.3 | 0.3 | Bladder | 1.1 | 2.3 |
| to Lung | | | Cancer 1023 | | |
| (OD04321) | | | | | |
| Lung Margin | 9.7 | 15.5 | Bladder | 0.3 | 1.0 |
| (OD04321) | | | Cancer | | |
| | | | A302173 | | |
| Normal Kidney | 18.4 | 28.1 | Bladder | 3.7 | 3.7 |
| | | | Cancer | | |
| | | | (OD04718-01) | | |
| Kidney Ca, | 6.9 | 6.7 | Bladder | 15.4 | 15.4 |
| Nuclear grade 2 | | | Normal | | |
| (OD04338) | | | Adjacent | | |
| | | | (OD04718-03) | | |
| Kidney Margin | 11.1 | 21.2 | Normal Ovary | 15.4 | 19.8 |
| (OD04338) | | | | | |
| Kidney Ca | 13.5 | 14.3 | Ovarian | 22.7 | 32.3 |
| Nuclear grade | | | Cancer 064008 | | |
| 1/2 (OD04339) | | | | | |
| Kidney Margin | 30.4 | 39.0 | Ovarian | 100.0 | 100.0 |
| (OD04339) | | | Cancer | | |
| | | | (OD04768-07) | | |
| Kidney Ca, Clear | 9.7 | 10.1 | Ovary Margin | 25.5 | 25.7 |
| cell type | | | (OD04768-08) | | |
| (OD04340) | | | | | |
| Kidney Margin | 31.2 | 33.0 | Normal | 13.9 | 16.7 |
| (OD04340) | | | Stomach | | |
| Kidney Ca, | 3.1 | 3.3 | Gastric Cancer | 3.5 | 3.9 |
| Nuclear grade 3 | | | 9060358 | | |
| (OD04348) | | | | | |
| Kidney Margin | 8.4 | 12.7 | Stomach | 3.0 | 3.7 |
| (OD04348) | | | Margin | | |
| | | | 9060359 | | |
| Kidney Cancer | 3.6 | 4.0 | Gastric Cancer | 6.7 | 6.3 |
| (OD04622-01) | | | 9060395 | | |
| Kidney Margin | 4.5 | 4.7 | Stomach | 6.1 | 7.8 |
| (OD04622-03) | | | Margin | | |
| | | | 9060394 | | |
| Kidney Cancer | 1.6 | 0.7 | Gastric Cancer | 9.2 | 13.8 |
| (OD04450-01) | | | 9060397 | | |
| Kidney Margin | 9.9 | 13.9 | Stomach | 3.3 | 3.0 |
| (OD04450-03) | | | Margin | | |
| | | | 9060396 | | |
| Kidney Cancer | 2.4 | 1.8 | Gastric Cancer | 4.7 | 5.0 |
| 8120607 | | | 064005 | | |

TABLE BCH
Panel 3D

| | Rel. Exp. (%) | Rel. Exp. (%) | | Rel. Exp. (%) | Rel. Exp. (%) |
|---|---|---|---|---|---|
| | Ag041b, Run | Ag41, Run | | Ag041b, Run | Ag41, Run |
| Tissue Name | 156897045 | 157938257 | Tissue Name | 156897045 | 157938257 |
| Daoy- | 0.4 | 0.2 | Ca Ski-Cervical | 0.0 | 0.0 |
| Medulloblastoma | | | epidermoid carcinoma | | |
| | | | (metastasis) | | |
| TE671- | 0.3 | 0.2 | ES-2-Ovarian clear | 0.0 | 0.0 |
| Medulloblastoma | | | cell carcinoma | | |
| D283 Med- | 0.1 | 0.3 | Ramos-Stimulated | 0.0 | 0.0 |
| Medulloblastoma | | | with PMA/ionomycin | | |
| | | | 6 h | | |
| PFSK-1-Primitive | 0.1 | 0.5 | Ramos-Stimulated | 0.0 | 0.0 |
| Neuroectodermal | | | with PMA/ionomycin | | |
| | | | 14 h | | |
| XF-498-CNS | 1.8 | 2.7 | MEG-01-Chronic | 0.0 | 0.3 |
| | | | myelogenous | | |
| | | | leukemia | | |
| | | | (megokaryoblast) | | |
| SNB-78-Glioma | 0.0 | 0.0 | Raji-Burkitt's | 0.0 | 0.0 |
| | | | lymphoma | | |
| SF-268- | 0.4 | 0.7 | Daudi-Burkitt's | 0.3 | 0.0 |
| Glioblastoma | | | lymphoma | | |
| T98G- | 1.9 | 2.0 | U266-B-cell | 0.0 | 0.0 |
| Glioblastoma | | | plasmacytoma | | |
| SK-N-SH- | 2.3 | 2.9 | CA46-Burkitt's | 0.1 | 0.0 |
| Neuroblastoma | | | lymphoma | | |
| (metastasis) | | | | | |
| SF-295- | 0.5 | 1.1 | RL-non-Hodgkin's | 0.0 | 0.0 |
| Glioblastoma | | | B-cell lymphoma | | |
| Cerebellum | 30.8 | 55.9 | JM1-pre-B-cell | 0.0 | 0.0 |
| | | | lymphoma | | |
| Cerebellum | 32.3 | 60.3 | Jurkat-T cell | 0.0 | 0.0 |
| | | | leukemia | | |
| NCI-H292- | 2.1 | 1.7 | TF-1- | 0.0 | 0.1 |
| Mucoepidermoid | | | Erythroleukemia | | |
| lung carcinoma | | | | | |
| DMS-114-Small | 6.0 | 6.4 | HUT 78-T-cell | 0.0 | 0.0 |
| cell lung cancer | | | lymphoma | | |
| DMS-79-Small | 100.0 | 100.0 | U937-Histiocytic | 0.2 | 0.0 |
| cell lung cancer | | | lymphoma | | |
| NCI-H146-Small | 0.0 | 0.3 | KU-812- | 0.8 | 0.8 |
| cell lung cancer | | | Myelogenous | | |
| | | | leukemia | | |
| NCI-H526-Small | 2.2 | 3.8 | 769-P-Clear cell | 0.5 | 0.1 |
| cell lung cancer | | | renal carcinoma | | |
| NCI-N417-Small | 2.7 | 4.5 | Caki-2-Clear cell | 1.4 | 1.4 |
| cell lung cancer | | | renal carcinoma | | |
| NCI-H82-Small | 0.1 | 0.3 | SW 839-Clear cell | 0.0 | 0.0 |
| cell lung cancer | | | renal carcinoma | | |
| NCI-H157- | 0.0 | 0.0 | G401-Wilms' tumor | 0.1 | 0.0 |
| Squamous cell | | | | | |
| lung cancer | | | | | |
| (metastasis) | | | | | |
| NCI-H1155- | 2.1 | 2.6 | Hs766T-Pancreatic | 1.1 | 1.2 |
| Large cell lung | | | carcinoma (LN | | |
| cancer | | | metastasis) | | |
| NCI-H1299- | 0.1 | 0.3 | CAPAN-1-Pancreatic | 7.5 | 13.0 |
| Large cell lung | | | adenocarcinoma (liver | | |
| cancer | | | metastasis) | | |
| NCI-H727-Lung | 0.0 | 0.0 | SU86.86-Pancreatic | 1.9 | 2.9 |
| carcinoid | | | carcinoma (liver | | |
| | | | metastasis) | | |
| NCI-UMC-11- | 0.1 | 0.0 | BxPC-3-Pancreatic | 5.0 | 7.0 |
| Lung carcinoid | | | adenocarcinoma | | |
| LX-1-Small cell | 1.3 | 1.4 | HPAC-Pancreatic | 0.4 | 0.4 |
| lung cancer | | | adenocarcinoma | | |
| Colo-205-Colon | 0.0 | 0.0 | MIA PaCa-2- | 3.6 | 4.0 |
| cancer | | | Pancreatic carcinoma | | |
| KM12-Colon | 0.0 | 0.0 | CFPAC-1-Pancreatic | 1.6 | 2.0 |
| cancer | | | ductal | | |
| | | | adenocarcinoma | | |
| KM20L2-Colon | 0.0 | 0.1 | PANC-1-Pancreatic | 20.3 | 22.1 |
| cancer | | | epithelioid ductal | | |
| | | | carcinoma | | |
| NCI-H716-Colon | 0.1 | 0.0 | T24-Bladder | 0.4 | 0.4 |
| cancer | | | carcinma (transitional | | |
| | | | cell) | | |
| SW-48-Colon | 0.1 | 0.0 | 5637-Bladder | 1.2 | 0.7 |
| adenocarcinoma | | | carcinoma | | |
| SW1116-Colon | 0.0 | 0.0 | HT-1197-Bladder | 0.0 | 0.2 |
| adenocarcinoma | | | carcinoma | | |
| LS 174T-Colon | 0.3 | 0.1 | UM-UC-3-Bladder | 0.1 | 0.0 |
| adenocarcinoma | | | carcinma (transitional | | |
| | | | cell) | | |
| SW-948-Colon | 0.0 | 0.0 | A204- | 2.3 | 3.5 |
| adenocarcinoma | | | Rhabdomyosarcoma | | |
| SW-480-Colon | 0.0 | 0.0 | HT-1080- | 0.2 | 0.4 |
| adenocarcinoma | | | Fibrosarcoma | | |
| NCI-SNU-5- | 0.5 | 0.6 | MG-63- | 9.3 | 20.9 |
| Gastric carcinoma | | | Osteosarcoma | | |
| KATO III-Gastric | 0.4 | 0.4 | SK-LMS-1- | 0.1 | 0.4 |
| carcinoma | | | Leiomyosarcoma | | |
| | | | (vulva) | | |
| NCI-SNU-16- | 0.1 | 0.0 | SJRH30- | 0.0 | 0.0 |
| Gastric carcinoma | | | Rhabdomyosarcoma | | |
| | | | (met to bone marrow) | | |
| NCI-SNU-1- | 0.2 | 0.0 | A431-Epidermoid | 0.4 | 0.3 |
| Gastric carcinoma | | | carcinoma | | |
| RF-1-Gastric | 0.3 | 0.0 | WM266-4- | 0.6 | 0.5 |
| adenocarcinoma | | | Melanoma | | |
| RF-48-Gastric | 0.0 | 0.0 | DU 145-Prostate | 0.0 | 0.0 |
| adenocarcinoma | | | carcinoma (brain | | |
| | | | metastasis) | | |
| MKN-45-Gastric | 0.7 | 0.4 | MDA-MB-468- | 26.4 | 30.4 |
| carcinoma | | | Breast | | |
| | | | adenocarcinoma | | |
| NCI-N87-Gastric | 0.2 | 0.1 | SCC-4-Squamous | 0.2 | 0.0 |
| carcinoma | | | cell carcinoma of | | |
| | | | tongue | | |
| OVCAR-5- | 1.7 | 1.7 | SCC-9-Squamous | 0.2 | 0.2 |
| Ovarian carcinoma | | | cell carcinoma of | | |
| | | | tongue | | |
| RL95-2-Uterine | 5.2 | 9.1 | SCC-15-Squamous | 0.6 | 0.3 |
| carcinoma | | | cell carcinoma of | | |
| | | | tongue | | |
| HelaS3-Cervical | 0.0 | 0.0 | CAL 27-Squamous | 0.0 | 0.1 |
| adenocarcinoma | | | cell carcinoma of | | |
| | | | tongue | | |

TABLE BCI
Panel 4D

| | Rel. Exp. (%) | | Rel. Exp. (%) |
|---|---|---|---|
| | Ag041b, Run | | Ag041b, Run |
| Tissue Name | 146087302 | Tissue Name | 146087302 |
| Secondary Th1 act | 2.1 | HUVEC IL-1beta | 1.6 |
| Secondary Th2 act | 0.9 | HUVEC IFN gamma | 11.0 |
| Secondary Tr1 act | 0.9 | HUVEC TNF alpha + IFN | 5.5 |
| | | gamma | |
| Secondary Th1 rest | 1.2 | HUVEC TNF alpha + IL4 | 2.9 |
| Secondary Th2 rest | 2.7 | HUVEC IL-11 | 5.8 |
| Secondary Tr1 rest | 3.0 | Lung Microvascular EC | 2.7 |
| | | none | |
| Primary Th1 act | 0.9 | Lung Microvascular EC | 0.8 |
| | | TNF alpha + IL-1beta | |
| Primary Th2 act | 1.6 | Microvascular Dermal EC | 2.4 |
| | | none | |
| Primary Tr1 act | 2.6 | Microsvasular Dermal EC | 2.0 |
| | | TNF alpha + IL-1beta | |
| Primary Th1 rest | 2.7 | Bronchial epithelium | 7.5 |
| | | TNF alpha + IL1beta | |
| Primary Th2 rest | 2.7 | Small airway epithelium | 2.3 |
| | | none | |
| Primary Tr1 rest | 0.8 | Small airway epithelium | 6.2 |
| | | TNF alpha + IL-1beta | |
| CD45RA CD4 | 2.4 | Coronery artery SMC rest | 6.6 |
| lymphocyte act | | | |
| CD45RO CD4 | 3.9 | Coronery artery SMC | 10.9 |
| lymphocyte act | | TNF alpha + IL-1beta | |
| CD8 lymphocyte act | 0.7 | Astrocytes rest | 10.2 |
| Secondary CD8 | 2.0 | Astrocytes TNF alpha + | 13.0 |
| lymphocyte rest | | IL-1beta | |
| Secondary CD8 | 0.0 | KU-812 (Basophil) rest | 2.3 |
| lymphocyte act | | | |
| CD4 lymphocyte none | 1.9 | KU-812 (Basophil) | 2.5 |
| | | PMA/ionomycin | |
| 2ry Th1/Th2/Tr1_anti- | 0.0 | CCD1106 (Keratinocytes) | 2.2 |
| CD95 CH11 | | none | |
| LAK cells rest | 3.3 | CCD1106 (Keratinocytes) | 0.8 |
| | | TNF alpha + IL-1beta | |
| LAK cells IL-2 | 1.3 | Liver cirrhosis | 3.9 |
| LAK cells IL-2 + IL-12 | 1.3 | Lupus kidney | 5.6 |
| LAK cells IL-2 + IFN | 2.2 | NCI-H292 none | 5.3 |
| gamma | | | |
| LAK cells IL-2 + IL-18 | 0.8 | NCI-H292 IL-4 | 6.2 |
| LAK cells | 1.3 | NCI-H292 IL-9 | 4.9 |
| PMA/ionomycin | | | |
| NK Cells IL-2 rest | 5.1 | NCI-H292 IL-13 | 9.2 |
| Two Way MLR 3 day | 1.8 | NCI-H292 IFN gamma | 7.1 |
| Two Way MLR 5 day | 0.2 | HPAEC none | 3.6 |
| Two Way MLR 7 day | 0.4 | HPAEC TNF alpha + IL- | 1.1 |
| | | 1beta | |
| PBMC rest | 0.9 | Lung fibroblast none | 74.2 |
| PBMC PWM | 1.6 | Lung fibroblast TNF alpha + | 33.4 |
| | | IL-1beta | |
| PBMC PHA-L | 0.3 | Lung fibroblast IL-4 | 100.0 |
| Ramos (B cell) none | 0.0 | Lung fibroblast IL-9 | 62.9 |
| Ramos (B cell) | 1.3 | Lung fibroblast IL-13 | 51.4 |
| ionomycin | | | |
| B lymphocytes PWM | 0.8 | Lung fibroblast IFN | 84.1 |
| | | gamma | |
| B lymphocytes CD40L | 0.4 | Dermal fibroblast | 6.7 |
| and IL-4 | | CCD1070 rest | |
| EOL-1 dbcAMP | 0.0 | Dermal fibroblast | 6.7 |
| | | CCD1070 TNF alpha | |
| EOL-1 dbcAMP | 0.0 | Dermal fibroblast | 15.3 |
| PMA/ionomycin | | CCD1070 IL-1beta | |
| Dendritic cells none | 12.2 | Dermal fibroblast IFN | 40.6 |
| | | gamma | |
| Dendritic cells LPS | 7.7 | Dermal fibroblast IL-4 | 83.5 |
| Dendritic cells anti- | 12.2 | IBD Colitis 2 | 3.9 |
| CD40 | | | |
| Monocytes rest | 6.3 | IBD Crohn's | 4.5 |
| Monocytes LPS | 0.7 | Colon | 20.4 |
| Macrophages rest | 6.7 | Lung | 11.5 |
| Macrophages LPS | 0.9 | Thymus | 41.5 |
| HUVEC none | 5.3 | Kidney | 4.1 |
| HUVEC starved | 6.3 | | |

CNS_neurodegeneration_v1.0 Summary: Ag041b This panel does not show differential expression of the NOV60a gene in Alzheimer's disease. However, this expression profile confirms the presence of this gene in the brain. Please see Panel 1.3D for discussion of utility of this gene in the central nervous system.

Panel 1 Summary: Ag41 Two experiments with the same probe and primer set produce results that are in reasonable agreement, with highest expression of the NOV60a gene in the mammary gland and the brain. Overall, this gene appears to express in normal tissues at higher levels than in cancer cell lines. This gene encodes a lynx1 homolog. Lynx1 is an endogenous toxin-like modulator of nicotinic acetylcholine receptors in the mammalian CNS. Activation of nicotinic receptors is associated with positive effect on schizophrenia and alzheimer's disease. Therefore, agents that block Ag41 action in the CNS are likely to have utility in the treatment of these, and related, disorders.

This gene also has high levels of expression in pancreas, adrenal, thyroid, pituitary, heart, skeletal muscle and liver. Therefore, therapeutic modulation of this gene product may be a treatment for endocrine and metabolic diseases, including obesity and Types 1 and 2 diabetes. Please note that two additional experiments with the same probe and primer set show low/undetectable levels of expression (CTs>35). (Data not shown.) The results indicate that there is a possibility of a probe failure.

References:

Miwa J M, Ibanez-Tallon I, Crabtree G W, Sanchez R, Sali A, Role L W, Heintz N. lynx1, an endogenous toxin-like modulator of nicotinic acetylcholine receptors in the mammalian CNS. Neuron May 1999;23(1):105-14

Elapid snake venom neurotoxins exert their effects through high-affinity interactions with specific neurotransmitter receptors. A novel murine gene, lynx1, is highly expressed in the brain and contains the cysteine-rich motif characteristic of this class of neurotoxins. Primary sequence and gene structure analyses reveal an evolutionary relationship between lynx1 and the Ly-6/neurotoxin gene family. lynx1 is expressed in large projection neurons in the hippocampus, cortex, and cerebellum. In cerebellar neurons, lynx1 protein is localized to a specific subdomain including the soma and proximal dendrites. lynx1 binding to brain sections correlates with the distribution of nAChRs, and application of lynx1 to Xenopus oocytes expressing nAChRs results in an increase in acetylcholine-evoked macroscopic currents. These results identify lynx1 as a novel protein modulator for nAChRs in vitro, which could have important implications in the regulation of cholinergic function in vivo.

Panel 1.1 Summary: Ag041b The NOV60a gene is expressed in most cell lines and normal tissues with a significantly higher level of expression in normal brain and heart compared to cancer cell lines on this panel. The results in this panel are consistent with expression in Panel 1. Please see Panel 1 for further discussion of utility of this gene in metabolic and cns diseases and cancer.

Panel 1.3D Summary: Ag041b Highest expression of the NOV60a gene is seen in the brain. Overall, this gene is expressed in most cell lines and normal tissues with a significantly higher level of expression in heart in addition to brain when compared to cancer cell lines on this panel. Please see Panel 1 for discussion of utility of this gene in the central nervous system.

Among metabolic tissues, this gene has a low level of expression in adipose, adult and fetal liver, adrenal, pituitary, fetal skeletal muscle, fetal and adult heart, thyroid and pancreas. Therefore, modulation of this gene product may be a treatment for endocrine and metabolic diseases, including obesity and Types 1 and 2 diabetes. In addition, this gene differentially expressed in fetal (CT value=29) vs adult skeletal muscle (CT value=35) and may be useful for the identification of the two sources of this tissue.

Panel 2D Summary: Ag041b/Ag41 The expression of the NOV60a gene was assessed in two independent runs on this panel with good concordance between runs. This protein is a good diagnostic marker and target in ovarian, renal and liver cancer as the cancer expressed this gene at a higher level than the normal adjacent tissue.

Panel 3D Summary: Ag041b/Ag41 Two experiments show expression of the NOV60a gene in cell lines derived from brain, lung, ovarian, renal, pancreatic, breast and osteosarcoma. Therefore, expression of this gene could be used as a diagnostic marker for the presence of these cancers. Furthermore, therapeutic modulation of the expression or function of this gene may be effective in the treatment of brain, lung, ovarian, renal, pancreatic, breast and osteosarcoma cancers.

Panel 4D Summary: Ag041b The NOV60a gene, a lynx1 homolog is expressed at moderate levels in untreated lung fibroblasts, lung fibroblasts activated with IL-4, IL-9 or IFN gamma, and dermal fibroblasts activated with IL-4 (CTs=30). Therefore, small molecules or therapeutic antibodies that antagonize the function of the NOV60a gene product may be useful to reduce or eliminate the symptoms in patients with chronic obstructive pulmonary disease, asthma, emphysema, or psoriasis.

NOV61:Adlican-like

Expression of gene NOV61 was assessed using the primer-probe sets Ag2933,Ag3370 and Ag3837, described in Tables BDA, BDB and BDC.

TABLE BDA
Probe Name Ag2933

| | | | Start | |
|---|---|---|---|---|
| Primers | Sequences | Length | Position | SEQ ID NO: |
| Forward | 5′-caccaccactaagccagaac-3′ | 20 | 4011 | 1237 |
| Probe | TET-5′-ttctcagtccaagaacatctcaaatatgt-3′-TAMRA | 29 | 4031 | 1238 |
| Reverse | 5′-ggattccccatgtaattcaag-3′ | 21 | 4083 | 1239 |

TABLE BDB
Probe Name Ag3370

| | | | Start | |
|---|---|---|---|---|
| Primers | Sequences | Length | Position | SEQ ID NO: |
| Forward | 5′-agctggattcttccaaacaga-3′ | 21 | 1327 | 1240 |
| Probe | TET-5′-tcacatgtatacatgctgccaaatgg-3′-TAMRA | 26 | 1375 | 1241 |
| Reverse | 5′-acctttgggatggaaagagtt-3′ | 21 | 1401 | 1242 |

TABLE BDC
Probe Name Ag3837

| | | | Start | |
|---|---|---|---|---|
| Primers | Sequences | Length | Position | SEQ ID NO: |
| Forward | 5′-acgagcttgaggatgtggat-3′ | 20 | 3725 | 1243 |
| Probe | TET-5′-ttttgtcctctgtgacagtctccaca-3′-TAMRA | 26 | 3758 | 1244 |
| Reverse | 5′-gcttcttcctggtgaaatgg-3′ | 20 | 3784 | 1245 |

CNS_neurodegeneration_v1.0 Summary: Ag2933/Ag3370 Expression of this gene is low/undetectable (CTs>35) across all of the samples on this panel (data not shown).

General_screening_panel_v1.4 Summary: Ag3370/Ag3837 The amp plots suggest that there were experimental difficulties with these runs (data not shown).

Panel 1.3D Summary: Ag2933 Expression of this gene is low/undetectable (CTs>35) across all of the samples on this panel (data not shown).

Panel 2D Summary: Ag2933 Expression of this gene is low/undetectable (CTs>35) across all of the samples on this panel (data not shown).

Panel 4D Summary: Ag2933/Ag3370 Expression of this gene is low/undetectable (CTs>35) across all of the samples on this panel (data not shown).

### BE. CG56781-01: Neuropsin Precursor

Expression of gene CG56781-01 was assessed using the primer-probe sets Ag3019 and Ag4966, described in Tables BEA and BEB.

TABLE BEA
Probe Name Ag3019

| | | | Start | |
|---|---|---|---|---|
| Primers | Sequences | Length | Position | SEQ ID NO: |
| Forward | 5′-aggatctgagcctgtgttcag-3′ | 21 | 714 | 1246 |
| Probe | TET-5′-cggagacccgctgtctacactaacgt-3′-TAMRA | 26 | 739 | 1247 |
| Reverse | 5′-ttcaatccactccaggtagtca-3′ | 22 | 768 | 1248 |

TABLE BEB
Probe Name Ag4966

| | | | Start | |
|---|---|---|---|---|
| Primers | Sequences | Length | Position | SEQ ID NO: |
| Forward | 5′-ccaccctcttcctcagag-3′ | 18 | 7 | 1249 |
| Probe | TET-5′-caccctgtgcaatccagccgtg-3′-TAMRA | 22 | 44 | 1250 |
| Reverse | 5′-acacctgcccacgctc-3′ | 16 | 89 | 1251 |

CNS_neurodegeneration_v1.0 Summary: Ag3019 The amp plot suggests that there were experimental difficulties with this run in one sample (data not shown). Given the lack of expression of this gene on the other panels the expression detected in the occipital cortex is likely artifactual.

Panel 1.3D Summary: Ag3019 Expression of this gene is low/undetectable (CTs>35) across all of the samples on this panel (data not shown).

Panel 4.1D Summary: Ag3019 Expression of this gene is low/undetectable (CTs>35) across all of the samples on this panel (data not shown).

Panel 4D Summary: Ag3019 Expression of this gene is low/undetectable (CTs>35) across all of the samples on this panel (data not shown).

Panel 5 Islet Summary: Ag3019 Expression of this gene is low/undetectable (CTs>35) across all of the samples on this panel (data not shown).

NOV63

Expression of gene NOV63 was assessed using the primer-probe sets Ag2261 and Ag3035, described in Tables BFA and BFB. Results of the RTQ-PCR runs are shown in Tables BFC, BFD, BFE and BFF.

TABLE BFA
Probe Name Ag2261

| | | | Start | |
|---|---|---|---|---|
| Primers | Sequences | Length | Position | SEQ ID NO: |
| Forward | 5′-ggatgactcgcctagcttct-3′ | 20 | 882 | 1252 |
| Probe | TET-5′-gccgtaggtgccaccgtgagaag-3′-TAMRA | 23 | 935 | 1253 |
| Reverse | 5′-agcagatgctctcgcagtt-3′ | 19 | 958 | 1254 |

TABLE BFB
Probe Name Ag3035

| | | | Start | |
|---|---|---|---|---|
| Primers | Sequences | Length | Position | SEQ ID NO: |
| Forward | 5′-acagcagcaagttcgtcaag-3′ | 20 | 527 | 1255 |
| Probe | TET-5′-agacggtcaagcaaggatctgcgag-3′-TAMRA | 25 | 559 | 1256 |
| Reverse | 5′-cacgaggttgttgtggaagt-3′ | 20 | 593 | 1257 |

TABLE BFC
Panel 1.3D

| | Rel. | Rel. | Rel. | | Rel. | Rel. | Rel. |
|---|---|---|---|---|---|---|---|
| | Exp. (%) | Exp. (%) | Exp. (%) | | Exp. (%) | Exp. (%) | Exp. (%) |
| | Ag2261, | Ag2261, | Ag3035, | | Ag2261, | Ag2261, | Ag3035, |
| Tissue | Run | Run | Run | Tissue | Run | Run | Run |
| Name | 150631675 | 152887692 | 167597764 | Name | 150631675 | 152887692 | 167597764 |
| Liver | 22.4 | 19.6 | 71.2 | Kidney | 2.1 | 0.0 | 2.7 |
| adenocarcinoma | | | | (fetal) | | | |
| Pancreas | 3.9 | 2.5 | 2.8 | Renal ca. | 0.0 | 0.0 | 0.0 |
| | | | | 786-0 | | | |
| Pancreatic | 5.3 | 3.5 | 9.5 | Renal ca. | 10.2 | 5.3 | 9.2 |
| ca. | | | | A498 | | | |
| CAPAN 2 | | | | | | | |
| Adrenal | 2.1 | 0.6 | 2.0 | Renal ca. | 0.0 | 0.0 | 0.0 |
| gland | | | | RXF 393 | | | |
| Thyroid | 7.0 | 9.8 | 3.9 | Renal ca. | 0.0 | 2.2 | 0.0 |
| | | | | ACHN | | | |
| Salivary | 1.9 | 2.1 | 4.2 | Renal ca. | 0.0 | 0.0 | 0.0 |
| gland | | | | UO-31 | | | |
| Pituitary | 1.0 | 2.2 | 6.7 | Renal ca. | 0.0 | 0.0 | 0.0 |
| gland | | | | TK-10 | | | |
| Brain | 6.8 | 4.9 | 10.8 | Liver | 0.0 | 0.0 | 0.0 |
| (fetal) | | | | | | | |
| Brain | 4.8 | 3.0 | 1.4 | Liver | 7.6 | 0.0 | 0.0 |
| (whole) | | | | (fetal) | | | |
| Brain | 4.6 | 5.3 | 1.5 | Liver ca. | 0.0 | 0.0 | 0.0 |
| (amygdala) | | | | (hepatoblast) | | | |
| | | | | HepG2 | | | |
| Brain | 1.6 | 1.6 | 2.0 | Lung | 14.3 | 15.8 | 9.2 |
| (cerebellum) | | | | | | | |
| Brain | 7.5 | 11.3 | 0.6 | Lung | 15.1 | 15.4 | 7.4 |
| (hippocampus) | | | | (fetal) | | | |
| Brain | 1.2 | 2.6 | 1.3 | Lung ca. | 1.6 | 0.0 | 0.0 |
| (substantia | | | | (small cell) | | | |
| nigra) | | | | LX-1 | | | |
| Brain | 2.5 | 1.7 | 2.6 | Lung ca. | 29.5 | 19.1 | 31.2 |
| (thalamus) | | | | (small cell) | | | |
| | | | | NCI-H69 | | | |
| Cerebral | 0.0 | 0.0 | 5.0 | Lung ca. | 11.0 | 5.1 | 37.4 |
| Cortex | | | | (s.cell var.) | | | |
| | | | | SHP-77 | | | |
| Spinal | 1.7 | 2.1 | 2.7 | Lung ca. | 0.0 | 0.0 | 0.0 |
| cord | | | | (large | | | |
| | | | | cell)NCI- | | | |
| | | | | H460 | | | |
| glio/astro | 0.0 | 0.0 | 0.0 | Lung ca. | 0.0 | 1.2 | 1.6 |
| U87-MG | | | | (non-sm. | | | |
| | | | | cell) A549 | | | |
| glio/astro | 55.1 | 50.3 | 42.9 | Lung ca. | 0.0 | 1.3 | 0.8 |
| U-118-MG | | | | (non- | | | |
| | | | | s.cell) | | | |
| | | | | NCI-H23 | | | |
| astrocytoma | 0.0 | 7.5 | 0.0 | Lung ca. | 0.0 | 1.7 | 0.0 |
| SW1783 | | | | (non- | | | |
| | | | | s.cell) | | | |
| | | | | HOP-62 | | | |
| neuro*; | 0.0 | 0.0 | 0.7 | Lung ca. | 8.0 | 8.3 | 7.3 |
| met SK-N- | | | | (non-s.cl) | | | |
| AS | | | | NCI-H522 | | | |
| astrocytoma | 1.9 | 4.7 | 9.9 | Lung ca. | 4.0 | 0.0 | 1.8 |
| SF-539 | | | | (squam.) | | | |
| | | | | SW 900 | | | |
| astrocytoma | 2.0 | 4.9 | 6.9 | Lung ca. | 15.8 | 10.2 | 58.2 |
| SNB-75 | | | | (squam.) | | | |
| | | | | NCI-H596 | | | |
| glioma | 6.7 | 2.4 | 3.7 | Mammary | 7.2 | 4.1 | 4.4 |
| SNB-19 | | | | gland | | | |
| glioma | 2.1 | 4.5 | 6.8 | Breast ca.* | 1.7 | 3.4 | 7.3 |
| U251 | | | | (pl.ef) | | | |
| | | | | MCF-7 | | | |
| glioma SF- | 10.0 | 0.6 | 4.6 | Breast ca.* | 23.2 | 19.6 | 19.2 |
| 295 | | | | (pl.ef) | | | |
| | | | | MDA- | | | |
| | | | | MB-231 | | | |
| Heart | 11.1 | 9.9 | 38.2 | Breast ca.* | 4.3 | 5.8 | 21.8 |
| (fetal) | | | | (pl.ef) | | | |
| | | | | T47D | | | |
| Heart | 4.9 | 6.0 | 15.2 | Breast ca. | 0.0 | 4.2 | 2.2 |
| | | | | BT-549 | | | |
| Skeletal | 100.0 | 100.0 | 85.3 | Breast ca. | 0.0 | 0.0 | 0.0 |
| muscle | | | | MDA-N | | | |
| (fetal) | | | | | | | |
| Skeletal | 5.5 | 8.4 | 39.8 | Ovary | 3.6 | 3.1 | 8.1 |
| muscle | | | | | | | |
| Bone | 0.0 | 0.0 | 0.7 | Ovarian | 1.1 | 1.0 | 5.6 |
| marrow | | | | ca. | | | |
| | | | | OVCAR-3 | | | |
| Thymus | 10.0 | 3.9 | 6.4 | Ovarian | 0.0 | 0.0 | 0.7 |
| | | | | ca. | | | |
| | | | | OVCAR-4 | | | |
| Spleen | 3.8 | 4.2 | 1.6 | Ovarian | 0.0 | 0.0 | 11.5 |
| | | | | ca. | | | |
| | | | | OVCAR-5 | | | |
| Lymph | 5.0 | 1.1 | 1.4 | Ovarian | 1.3 | 4.3 | 4.1 |
| node | | | | ca. | | | |
| | | | | OVCAR-8 | | | |
| Colorectal | 3.4 | 5.4 | 6.8 | Ovarian | 0.0 | 0.0 | 8.1 |
| | | | | ca. | | | |
| | | | | IGROV-1 | | | |
| Stomach | 6.0 | 15.4 | 3.1 | Ovarian | 7.5 | 16.0 | 100.0 |
| | | | | ca.* | | | |
| | | | | (ascites) | | | |
| | | | | SK-OV-3 | | | |
| Small | 15.9 | 18.7 | 2.3 | Uterus | 17.8 | 15.1 | 9.9 |
| intestine | | | | | | | |
| Colon ca. | 24.3 | 15.3 | 11.6 | Placenta | 4.6 | 8.2 | 2.1 |
| SW480 | | | | | | | |
| Colon ca.* | 0.0 | 0.0 | 2.1 | Prostate | 3.6 | 5.3 | 0.6 |
| SW620(SW480 | | | | | | | |
| met) | | | | | | | |
| Colon ca. | 0.0 | 0.0 | 0.0 | Prostate | 1.7 | 1.5 | 6.1 |
| HT29 | | | | ca.* (bone | | | |
| | | | | met)PC-3 | | | |
| Colon ca. | 3.8 | 0.6 | 3.3 | Testis | 21.9 | 14.6 | 1.6 |
| HCT-116 | | | | | | | |
| Colon ca. | 0.0 | 0.8 | 0.3 | Melanoma | 3.1 | 4.7 | 1.4 |
| CaCo-2 | | | | Hs688(A).T | | | |
| Colon ca. | 2.3 | 0.0 | 1.6 | Melanoma* | 0.4 | 1.3 | 0.0 |
| tissue(ODO3866) | | | | (met) | | | |
| | | | | Hs688(B).T | | | |
| Colon ca. | 0.0 | 0.0 | 1.6 | Melanoma | 0.0 | 0.0 | 0.0 |
| HCC-2998 | | | | UACC-62 | | | |
| Gastric | 16.7 | 14.9 | 15.3 | Melanoma | 0.0 | 0.0 | 0.0 |
| ca.* (liver | | | | M14 | | | |
| met) NCI- | | | | | | | |
| N87 | | | | | | | |
| Bladder | 1.6 | 3.2 | 3.0 | Melanoma | 0.0 | 0.0 | 0.0 |
| | | | | LOX | | | |
| | | | | IMVI | | | |
| Trachea | 24.3 | 33.7 | 5.7 | Melanoma* | 0.0 | 2.0 | 0.7 |
| | | | | (met) | | | |
| | | | | SK-MEL-5 | | | |
| Kidney | 0.0 | 0.0 | 0.0 | Adipose | 6.7 | 7.2 | 21.2 |

TABLE BFD
Panel 2D

| | Rel. Exp. (%) | Rel. Exp. (%) | | Rel. Exp. (%) | Rel. Exp. (%) |
|---|---|---|---|---|---|
| | Ag2261, Run | Ag2261, Run | | Ag2261, Run | Ag2261, Run |
| Tissue Name | 150811744 | 152887693 | Tissue Name | 150811744 | 152887693 |
| Normal Colon | 19.1 | 19.8 | Kidney | 2.4 | 0.0 |
| | | | Margin | | |
| | | | 8120608 | | |
| CC Well to Mod | 0.0 | 5.8 | Kidney Cancer | 14.6 | 7.3 |
| Diff (ODO3866) | | | 8120613 | | |
| CC Margin | 19.5 | 12.5 | Kidney | 4.8 | 1.5 |
| (ODO3866) | | | Margin | | |
| | | | 8120614 | | |
| CC Gr.2 | 3.8 | 1.4 | Kidney Cancer | 0.0 | 0.0 |
| rectosigmoid | | | 9010320 | | |
| (ODO3868) | | | | | |
| CC Margin | 2.6 | 5.1 | Kidney | 0.0 | 0.0 |
| (ODO3868) | | | Margin | | |
| | | | 9010321 | | |
| CC Mod Diff | 6.0 | 2.9 | Normal Uterus | 9.7 | 2.8 |
| (ODO3920) | | | | | |
| CC Margin | 23.8 | 6.4 | Uterus Cancer | 85.9 | 41.5 |
| (ODO3920) | | | 064011 | | |
| CC Gr.2 ascend | 9.3 | 2.2 | Normal | 15.2 | 7.3 |
| colon | | | Thyroid | | |
| (ODO3921) | | | | | |
| CC Margin | 16.8 | 11.7 | Thyroid | 0.0 | 3.0 |
| (ODO3921) | | | Cancer | | |
| | | | 064010 | | |
| CC from Partial | 2.4 | 0.0 | Thyroid | 1.9 | 1.2 |
| Hepatectomy | | | Cancer | | |
| (ODO4309) | | | A302152 | | |
| Mets | | | | | |
| Liver Margin | 2.6 | 0.0 | Thyroid | 2.6 | 2.8 |
| (ODO4309) | | | Margin | | |
| | | | A302153 | | |
| Colon mets to | 7.9 | 4.5 | Normal Breast | 16.2 | 2.7 |
| lung (OD04451- | | | | | |
| 01) | | | | | |
| Lung Margin | 11.3 | 12.9 | Breast Cancer | 78.5 | 29.7 |
| (OD04451-02) | | | (OD04566) | | |
| Normal Prostate | 6.3 | 2.6 | Breast Cancer | 37.6 | 23.8 |
| 6546-1 | | | (OD04590-01) | | |
| Prostate Cancer | 17.8 | 7.3 | Breast Cancer | 100.0 | 24.5 |
| (OD04410) | | | Mets | | |
| | | | (OD04590-03) | | |
| Prostate Margin | 10.7 | 7.4 | Breast Cancer | 94.0 | 45.4 |
| (OD04410) | | | Metastasis | | |
| | | | (OD04655-05) | | |
| Prostate Cancer | 4.7 | 4.4 | Breast Cancer | 25.7 | 24.8 |
| (OD04720-01) | | | 064006 | | |
| Prostate Margin | 13.9 | 5.6 | Breast Cancer | 23.2 | 7.1 |
| (OD04720-02) | | | 1024 | | |
| Normal Lung | 36.6 | 14.3 | Breast Cancer | 33.0 | 7.5 |
| 061010 | | | 9100266 | | |
| Lung Met to | 1.0 | 0.0 | Breast Margin | 7.6 | 7.6 |
| Muscle | | | 9100265 | | |
| (ODO4286) | | | | | |
| Muscle Margin | 31.0 | 38.2 | Breast Cancer | 13.9 | 0.9 |
| (ODO4286) | | | A209073 | | |
| Lung Malignant | 81.8 | 100.0 | Breast Margin | 2.5 | 0.0 |
| Cancer | | | A2090734 | | |
| (OD03126) | | | | | |
| Lung Margin | 35.8 | 18.2 | Normal Liver | 0.0 | 0.0 |
| (OD03126) | | | | | |
| Lung Cancer | 57.0 | 39.5 | Liver Cancer | 0.0 | 0.0 |
| (OD04404) | | | 064003 | | |
| Lung Margin | 9.4 | 11.8 | Liver Cancer | 4.8 | 1.7 |
| (OD04404) | | | 1025 | | |
| Lung Cancer | 37.1 | 42.0 | Liver Cancer | 7.1 | 0.0 |
| (OD04565) | | | 1026 | | |
| Lung Margin | 22.7 | 9.3 | Liver Cancer | 4.8 | 0.0 |
| (OD04565) | | | 6004-T | | |
| Lung Cancer | 5.3 | 6.4 | Liver Tissue | 4.4 | 1.8 |
| (OD04237-01) | | | 6004-N | | |
| Lung Margin | 78.5 | 32.8 | Liver Cancer | 0.0 | 6.0 |
| (OD04237-02) | | | 6005-T | | |
| Ocular Mel Met | 0.0 | 0.0 | Liver Tissue | 0.0 | 1.8 |
| to Liver | | | 6005-N | | |
| (ODO4310) | | | | | |
| Liver Margin | 2.4 | 0.0 | Normal | 2.4 | 3.0 |
| (ODO4310) | | | Bladder | | |
| Melanoma Mets | 13.0 | 0.0 | Bladder | 8.5 | 4.9 |
| to Lung | | | Cancer 1023 | | |
| (OD04321) | | | | | |
| Lung Margin | 96.6 | 50.0 | Bladder | 17.0 | 11.8 |
| (OD04321) | | | Cancer | | |
| | | | A302173 | | |
| Normal Kidney | 0.0 | 0.0 | Bladder | 10.0 | 5.7 |
| | | | Cancer | | |
| | | | (OD04718-01) | | |
| Kidney Ca, | 0.0 | 0.0 | Bladder | 19.3 | 27.5 |
| Nuclear grade 2 | | | Normal | | |
| (OD04338) | | | Adjacent | | |
| | | | (OD04718-03) | | |
| Kidney Margin | 4.0 | 4.6 | Normal Ovary | 13.6 | 12.4 |
| (OD04338) | | | | | |
| Kidney Ca | 0.0 | 3.3 | Ovarian | 37.9 | 2.1 |
| Nuclear grade | | | Cancer | | |
| 1/2 (OD04339) | | | 064008 | | |
| Kidney Margin | 18.7 | 0.0 | Ovarian | 18.4 | 3.7 |
| (OD04339) | | | Cancer | | |
| | | | (OD04768-07) | | |
| Kidney Ca, Clear | 8.8 | 11.7 | Ovary Margin | 28.3 | 12.2 |
| cell type | | | (OD04768-08) | | |
| (OD04340) | | | | | |
| Kidney Margin | 0.0 | 2.0 | Normal | 48.3 | 17.3 |
| (OD04340) | | | Stomach | | |
| Kidney Ca, | 3.5 | 4.0 | Gastric Cancer | 0.0 | 0.0 |
| Nuclear grade 3 | | | 9060358 | | |
| (OD04348) | | | | | |
| Kidney Margin | 2.0 | 1.7 | Stomach | 9.9 | 3.0 |
| (OD04348) | | | Margin | | |
| | | | 9060359 | | |
| Kidney Cancer | 9.3 | 0.0 | Gastric Cancer | 20.7 | 10.4 |
| (OD04622-01) | | | 9060395 | | |
| Kidney Margin | 0.0 | 6.3 | Stomach | 10.0 | 12.2 |
| (OD04622-03) | | | Margin | | |
| | | | 9060394 | | |
| Kidney Cancer | 0.0 | 0.0 | Gastric Cancer | 8.7 | 1.5 |
| (OD04450-01) | | | 9060397 | | |
| Kidney Margin | 0.0 | 0.0 | Stomach | 7.5 | 6.2 |
| (OD04450-03) | | | Margin | | |
| | | | 9060396 | | |
| Kidney Cancer | 0.0 | 0.7 | Gastric Cancer | 10.7 | 4.8 |
| 8120607 | | | 064005 | | |

TABLE BFE
Panel 4.1D

| | Rel. Exp. (%) | | Rel. Exp. (%) |
|---|---|---|---|
| | Ag3035, Run | | Ag3035, Run |
| Tissue Name | 190944495 | Tissue Name | 190944495 |
| Secondary Th1 act | 0.0 | HUVEC IL-1beta | 1.7 |
| Secondary Th2 act | 0.0 | HUVEC IFN gamma | 0.8 |
| Secondary Tr1 act | 0.0 | HUVEC TNF alpha + IFN | 0.2 |
| | | gamma | |
| Secondary Th1 rest | 0.0 | HUVEC TNF alpha + IL4 | 0.6 |
| Secondary Th2 rest | 0.0 | HUVEC IL-11 | 1.1 |
| Secondary Tr1 rest | 0.0 | Lung Microvascular EC | 2.7 |
| | | none | |
| Primary Th1 act | 0.0 | Lung Microvascular EC | 0.6 |
| | | TNF alpha + IL-1beta | |
| Primary Th2 act | 0.0 | Microvascular Dermal EC | 3.8 |
| | | none | |
| Primary Tr1 act | 0.0 | Microsvasular Dermal EC | 1.2 |
| | | TNF alpha + IL-1beta | |
| Primary Th1 rest | 0.0 | Bronchial epithelium | 3.7 |
| | | TNF alpha + IL1beta | |
| Primary Th2 rest | 0.0 | Small airway epithelium | 1.9 |
| | | none | |
| Primary Tr1 rest | 0.0 | Small airway epithelium | 4.0 |
| | | TNF alpha + IL-1beta | |
| CD45RA CD4 | 0.0 | Coronery artery SMC rest | 0.2 |
| lymphocyte act | | | |
| CD45RO CD4 | 0.0 | Coronery artery SMC | 0.0 |
| lymphocyte act | | TNF alpha + IL-1beta | |
| CD8 lymphocyte act | 0.0 | Astrocytes rest | 2.4 |
| Secondary CD8 | 0.0 | Astrocytes TNF alpha + | 1.3 |
| lymphocyte rest | | IL-1beta | |
| Secondary CD8 | 0.0 | KU-812 (Basophil) rest | 0.0 |
| lymphocyte act | | | |
| CD4 lymphocyte none | 0.0 | KU-812 (Basophil) | 2.1 |
| | | PMA/ionomycin | |
| 2ry Th1/Th2/Tr1_anti- | 0.0 | CCD1106 (Keratinocytes) | 22.2 |
| CD95 CH11 | | none | |
| LAK cells rest | 0.0 | CCD1106 (Keratinocytes) | 18.8 |
| | | TNF alpha + IL-1beta | |
| LAK cells IL-2 | 0.0 | Liver cirrhosis | 0.7 |
| LAK cells IL-2 + IL-12 | 0.0 | NCI-H292 none | 0.4 |
| LAK cells IL-2 + IFN | 0.0 | NCI-H292 IL-4 | 1.5 |
| gamma | | | |
| LAK cells IL-2 + IL-18 | 0.0 | NCI-H292 IL-9 | 2.0 |
| LAK cells | 11.0 | NCI-H292 IL-13 | 1.4 |
| PMA/ionomycin | | | |
| NK Cells IL-2 rest | 0.0 | NCI-H292 IFN gamma | 1.5 |
| Two Way MLR 3 day | 0.0 | HPAEC none | 3.1 |
| Two Way MLR 5 day | 0.0 | HPAEC TNF alpha + IL- | 0.5 |
| | | 1beta | |
| Two Way MLR 7 day | 0.0 | Lung fibroblast none | 6.2 |
| PBMC rest | 0.0 | Lung fibroblast TNF alpha + | 2.1 |
| | | IL-1beta | |
| PBMC PWM | 0.5 | Lung fibroblast IL-4 | 4.2 |
| PBMC PHA-L | 0.4 | Lung fibroblast IL-9 | 8.3 |
| Ramos (B cell) none | 0.0 | Lung fibroblast IL-13 | 4.0 |
| Ramos (B cell) | 0.0 | Lung fibroblast IFN | 8.1 |
| ionomycin | | gamma | |
| B lymphocytes PWM | 0.0 | Dermal fibroblast | 0.4 |
| | | CCD1070 rest | |
| B lymphocytes CD40L | 0.0 | Dermal fibroblast | 0.9 |
| and IL-4 | | CCD1070 TNF alpha | |
| EOL-1 dbcAMP | 0.0 | Dermal fibroblast | 2.9 |
| | | CCD1070 IL-1beta | |
| EOL-1 dbcAMP | 1.0 | Dermal fibroblast IFN | 5.8 |
| PMA/ionomycin | | gamma | |
| Dendritic cells none | 0.0 | Dermal fibroblast IL-4 | 17.2 |
| Dendritic cells LPS | 0.0 | Dermal Fibroblasts rest | 4.8 |
| Dendritic cells anti- | 0.0 | Neutrophils TNFa + LPS | 1.0 |
| CD40 | | | |
| Monocytes rest | 0.0 | Neutrophils rest | 2.2 |
| Monocytes LPS | 0.6 | Colon | 2.6 |
| Macrophages rest | 0.0 | Lung | 8.8 |
| Macrophages LPS | 0.0 | Thymus | 17.1 |
| HUVEC none | 2.4 | Kidney | 100.0 |
| HUVEC starved | 8.8 | | |

TABLE BFF
Panel 4D

| | Rel. | Rel. | | Rel. | Rel. |
|---|---|---|---|---|---|
| | Exp. (%) | Exp. (%) | | Exp. (%) | Exp. (%) |
| | Ag2261, | Ag3035, | | Ag2261, | Ag3035, |
| | Run | Run | | Run | Run |
| Tissue Name | 152887762 | 165242424 | Tissue Name | 152887762 | 165242424 |
| Secondary Th1 act | 0.0 | 2.1 | HUVEC IL-1beta | 0.0 | 1.7 |
| Secondary Th2 act | 0.0 | 0.0 | HUVEC IFN | 3.7 | 11.5 |
| | | | gamma | | |
| Secondary Tr1 act | 0.0 | 4.2 | HUVEC TNF | 0.0 | 3.1 |
| | | | alpha + IFN | | |
| | | | gamma | | |
| Secondary Th1 rest | 0.0 | 0.0 | HUVEC TNF | 4.3 | 5.1 |
| | | | alpha + IL4 | | |
| Secondary Th2 rest | 0.0 | 2.3 | HUVEC IL-11 | 4.0 | 11.2 |
| Secondary Tr1 rest | 0.0 | 0.0 | Lung | 7.2 | 8.1 |
| | | | Microvascular EC | | |
| | | | none | | |
| Primary Th1 act | 0.0 | 0.0 | Lung | 0.0 | 0.0 |
| | | | Microvascular EC | | |
| | | | TNF alpha + IL- | | |
| | | | 1beta | | |
| Primary Th2 act | 0.0 | 0.0 | Microvascular | 8.4 | 14.5 |
| | | | Dermal EC none | | |
| Primary Tr1 act | 0.0 | 0.0 | Microsvasular | 0.0 | 2.2 |
| | | | Dermal EC | | |
| | | | TNF alpha + IL- | | |
| | | | 1beta | | |
| Primary Th1 rest | 0.0 | 0.0 | Bronchial | 0.0 | 16.3 |
| | | | epithelium | | |
| | | | TNF alpha + | | |
| | | | IL1beta | | |
| Primary Th2 rest | 0.0 | 0.0 | Small airway | 5.9 | 18.8 |
| | | | epithelium none | | |
| Primary Tr1 rest | 0.0 | 0.0 | Small airway | 24.3 | 58.6 |
| | | | epithelium | | |
| | | | TNF alpha + IL- | | |
| | | | 1beta | | |
| CD45RA CD4 | 0.0 | 0.0 | Coronery artery | 0.0 | 2.0 |
| lymphocyte act | | | SMC rest | | |
| CD45RO CD4 | 0.0 | 0.0 | Coronery artery | 0.0 | 0.0 |
| lymphocyte act | | | SMC TNF | | |
| | | | alpha + IL-1beta | | |
| CD8 lymphocyte | 0.0 | 0.0 | Astrocytes rest | 3.3 | 13.5 |
| act | | | | | |
| Secondary CD8 | 0.0 | 0.7 | Astrocytes | 0.0 | 8.6 |
| lymphocyte rest | | | TNF alpha + IL- | | |
| | | | 1beta | | |
| Secondary CD8 | 1.6 | 0.0 | KU-812 | 0.0 | 0.0 |
| lymphocyte act | | | (Basophil) rest | | |
| CD4 lymphocyte | 0.0 | 0.0 | KU-812 | 0.0 | 9.7 |
| none | | | (Basophil) | | |
| | | | PMA/ionomycin | | |
| 2ry | 0.0 | 1.4 | CCD1106 | 47.3 | 100.0 |
| Th1/Th2/Tr1_anti- | | | (Keratinocytes) | | |
| CD95 CH11 | | | none | | |
| LAK cells rest | 3.5 | 0.0 | CCD1106 | 9.0 | 53.6 |
| | | | (Keratinocytes) | | |
| | | | TNF alpha + IL- | | |
| | | | 1beta | | |
| LAK cells IL-2 | 0.0 | 0.0 | Liver cirrhosis | 32.8 | 9.4 |
| LAK cells IL-2 + | 0.0 | 0.0 | Lupus kidney | 0.0 | 1.6 |
| IL-12 | | | | | |
| LAK cells IL- | 0.0 | 4.0 | NCI-H292 none | 3.8 | 3.4 |
| 2 + IFN gamma | | | | | |
| LAK cells IL-2 + | 0.0 | 0.0 | NCI-H292 IL-4 | 8.0 | 19.5 |
| IL-18 | | | | | |
| LAK cells | 26.1 | 50.7 | NCI-H292 IL-9 | 0.0 | 4.2 |
| PMA/ionomycin | | | | | |
| NK Cells IL-2 rest | 0.0 | 0.0 | NCI-H292 IL-13 | 13.8 | 7.0 |
| Two Way MLR 3 | 0.0 | 0.0 | NCI-H292 IFN | 16.2 | 5.7 |
| day | | | gamma | | |
| Two Way MLR 5 | 0.0 | 0.0 | HPAEC none | 6.7 | 30.1 |
| day | | | | | |
| Two Way MLR 7 | 0.0 | 0.0 | HPAEC TNF | 0.0 | 0.0 |
| day | | | alpha + IL-1beta | | |
| PBMC rest | 0.0 | 0.0 | Lung fibroblast | 7.6 | 42.0 |
| | | | none | | |
| PBMC PWM | 0.0 | 0.0 | Lung fibroblast | 3.1 | 6.3 |
| | | | TNF alpha + IL-1 | | |
| | | | beta | | |
| PBMC PHA-L | 0.0 | 0.0 | Lung fibroblast | 4.3 | 34.2 |
| | | | IL-4 | | |
| Ramos (B cell) | 0.0 | 0.0 | Lung fibroblast | 12.7 | 27.5 |
| none | | | IL-9 | | |
| Ramos (B cell) | 0.0 | 0.0 | Lung fibroblast | 6.8 | 19.9 |
| ionomycin | | | IL-13 | | |
| B lymphocytes | 0.0 | 0.0 | Lung fibroblast | 30.4 | 51.1 |
| PWM | | | IFN gamma | | |
| B lymphocytes | 3.1 | 0.0 | Dermal fibroblast | 0.0 | 2.8 |
| CD40L and IL-4 | | | CCD1070 rest | | |
| EOL-1 dbcAMP | 0.0 | 0.0 | Dermal fibroblast | 5.2 | 19.6 |
| | | | CCD1070 TNF | | |
| | | | alpha | | |
| EOL-1 dbcAMP | 3.5 | 2.7 | Dermal fibroblast | 0.0 | 2.0 |
| PMA/ionomycin | | | CCD1070 IL-1 | | |
| | | | beta | | |
| Dendritic cells | 0.0 | 0.0 | Dermal fibroblast | 28.5 | 32.1 |
| none | | | IFN gamma | | |
| Dendritic cells LPS | 0.0 | 0.0 | Dermal fibroblast | 42.9 | 91.4 |
| | | | IL-4 | | |
| Dendritic cells anti- | 0.0 | 0.0 | IBD Colitis 2 | 2.2 | 5.5 |
| CD40 | | | | | |
| Monocytes rest | 0.0 | 0.0 | IBD Crohn's | 3.1 | 9.6 |
| Monocytes LPS | 0.0 | 0.0 | Colon | 100.0 | 58.6 |
| Macrophages rest | 0.0 | 0.0 | Lung | 36.3 | 26.1 |
| Macrophages LPS | 0.0 | 0.0 | Thymus | 0.0 | 0.0 |
| HUVEC none | 0.0 | 17.7 | Kidney | 4.0 | 33.0 |
| HUVEC starved | 17.4 | 51.1 | | | |

Panel 1.3D Summary: Ag2261 The NOV63 gene is expressed at moderate levels in a number of metabolic tissues, with highest overall expression seen in fetal skeletal muscle (CTs=30.4-31.8). The higher levels of expression in fetal skeletal muscle when compared to adult skeletal muscle suggests that the protein product encoded by the 88091010_EXT gene may be useful in treating muscular dystrophy, Lesch-Nyhan syndrome, myasthenia gravis and other conditions that result in weak or dystrophic muscle. This gene is also expressed in adipose, thyroid and heart. Since biologic cross-talk between adipose and thyroid is a component of some forms of obesity, this gene product may be a protein therapeutic for the treatment of metabolic disease, including obesity and Type 2 diabetes.

Ag3035 This probe/primer set recognizes a distinct portion of this gene and shows a distinctive expression pattern when compared to Ag2261. This observation may indicate that the probe/primer sets can distinguish splice variants of this gene. In contrast to the results obtained with Ag 2261, expression of this gene is highest in an ovarian cancer cell line (CT=30.6). As is the case for Ag2261, expression of this gene using Ag3035 also shows relatively high levels in fetal skeletal muscle. However, in addition, Ag3035 shows increased levels of this gene in adult skeletal muscle as well as in adult and fetal heart. Most other expression is similar using both probe/primer sets. Please see Ag2261 for additional information.

Panel 2D Summary: Ag2261 The expression of this gene was assessed in two independent runs on panel 2D. This is consistently expressed in samples of breast cancer, uterine cancer and lung cancer when compared to their respective normal adjacent tissue controls. Thus, the expression of this gene could be used to distinguish breast cancer, lung cancer or uterine cancer from their normal tissues. Moreover, therapeutic modulation of this gene, through the use of small molecule drugs, antibodies or protein therapeutics might be of use in the treatment of breast, lung or uterine cancer.

Panel 4.1D Summary: Ag3035 This probe/primer set recognizes a distinct portion of this gene and shows a distinctive expression pattern when compared to Ag2261 in Panel 4D. This observation may indicate that the probe/primer sets can distinguish splice variants of this gene. In contrast to the results obtained with Ag2261, expression of this gene is highest in kidney (CT=30.6). Most other expression is similar using both probe/primer sets. The NOV63 gene, a WNT-14 homolog is also expressed at moderate to low levels in several unstimulated or cytokine-activated keratinocyte and lung and dermal fibroblast preparations (CT range 29-34). Thus, the NOV63 gene may be useful as a protein therapeutic that reduces or eliminates the symptoms of chronic obstructive pulmonary disease, asthma, emphysema, or psoriasis. In addition, due to its known effects on development of vertebrate joints, the protein encoded by the NOV63 gene may also reduce or eliminate the symptoms of osetoarthritis (See Hartmann and Tabin, 2001).

References:

Christine Hartmann and Clifford J. Tabin Wnt-14 Plays a Pivotal Role in Inducing Synovial Joint Formation in the Developing Appendicular Skeleton Cell, Vol 104, 341-351, February 2001

The long bones of the vertebrate appendicular skeleton arise from initially continuous condensations of mesenchymal cells that subsequently segment and cavitate to form discrete elements separated by synovial joints. Little is known, however, about the molecular mechanisms of joint formation. We present evidence that Wnt-14 plays a central role in initiating synovial joint formation in the chick limb. Wnt-14 is expressed in joint-forming regions prior to the segmentation of the cartilage elements, and local misexpression of Wnt-14 induces morphological and molecular changes characteristic of the first steps of joint formation. Induction of an ectopic joint-like region by Wnt-14 suppresses the formation of the immediately adjacent endogenous joint, potentially providing insight into the spacing of joints.

Panel 4D Summary: Ag2261 The NOV63 transcript is expressed at low levels in colon (CT=33.5). Low but significant levels of expression are also found in the lung, keratinocytes and dermal fibroblast. Thus, this transcript could be used as a marker for thymic, lung and skin tissues. The putative Wnt-14 molecule encoded by this transcript may play an important role in the normal homeostasis of these tissues. Therapeutics designed with the protein encoded by this transcript could be important for maintaining or restoring normal function to these organs during inflammation.

NOV64

Expression of gene NOV64 was assessed using the primer-probe set Ag3043, described in Table BGA. Results of the RTQ-PCR runs are shown in Tables BGB, BGC and BGD.

TABLE BGA
Probe Name Ag3043

| | | | Start | |
|---|---|---|---|---|
| Primers | Sequences | Length | Position | SEQ ID NO: |
| Forward | 5′-cctgtatgaggaagtcgatgag-3′ | 22 | 868 | 1258 |
| Probe | TET-5′-aggtcattcacgtcccctctcctg-3′-TAMRA | 24 | 900 | 1259 |
| Reverse | 5′-gatacgagtccgtcttcctttc-3′ | 22 | 932 | 1260 |

TABLE BGB
CNS_neurodegeneration_v1.0

| | Rel. Exp. (%) Ag3043, | | Rel. Exp. (%) Ag3043, |
|---|---|---|---|
| Tissue Name | Run 211012232 | Tissue Name | Run 211012232 |
| AD 1 Hippo | 23.0 | Control (Path) 3 | 10.2 |
| | | Temporal Ctx | |
| AD 2 Hippo | 32.8 | Control (Path) 4 | 28.3 |
| | | Temporal Ctx | |
| AD 3 Hippo | 12.3 | AD 1 Occipital Ctx | 20.2 |
| AD 4 Hippo | 11.2 | AD 2 Occipital Ctx | 0.0 |
| | | (Missing) | |
| AD 5 Hippo | 88.9 | AD 3 Occipital Ctx | 13.1 |
| AD 6 Hippo | 73.2 | AD 4 Occipital Ctx | 26.2 |
| Control 2 Hippo | 31.6 | AD 5 Occipital Ctx | 47.6 |
| Control 4 Hippo | 15.0 | AD 6 Occipital Ctx | 27.5 |
| Control (Path) 3 | 13.1 | Control 1 Occipital | 8.7 |
| Hippo | | Ctx | |
| AD 1 Temporal Ctx | 32.3 | Control 2 Occipital | 57.0 |
| | | Ctx | |
| AD 2 Temporal Ctx | 44.4 | Control 3 Occipital | 25.5 |
| | | Ctx | |
| AD 3 Temporal Ctx | 15.4 | Control 4 Occipital | 11.9 |
| | | Ctx | |
| AD 4 Temporal Ctx | 29.1 | Control (Path) 1 | 68.8 |
| | | Occipital Ctx | |
| AD 5 Inf Temporal | 100.0 | Control (Path) 2 | 14.1 |
| Ctx | | Occipital Ctx | |
| AD 5 Sup Temporal | 54.3 | Control (Path) 3 | 6.7 |
| Ctx | | Occipital Ctx | |
| AD 6 Inf Temporal | 72.7 | Control (Path) 4 | 17.3 |
| Ctx | | Occipital Ctx | |
| AD 6 Sup Temporal | 58.2 | Control 1 Parietal | 12.9 |
| Ctx | | Ctx | |
| Control 1 Temporal | 12.0 | Control 2 Parietal | 47.0 |
| Ctx | | Ctx | |
| Control 2 Temporal | 37.1 | Control 3 Parietal | 19.6 |
| Ctx | | Ctx | |
| Control 3 Temporal | 21.0 | Control (Path) 1 | 62.4 |
| Ctx | | Parietal Ctx | |
| Control 3 Temporal | 13.5 | Control (Path) 2 | 24.8 |
| Ctx | | Parietal Ctx | |
| Control (Path) 1 | 56.6 | Control (Path) 3 | 6.0 |
| Temporal Ctx | | Parietal Ctx | |
| Control (Path) 2 | 40.3 | Control (Path) 4 | 46.3 |
| Temporal Ctx | | Parietal Ctx | |

TABLE BGC
Panel 1.3D

| | Rel. Exp. (%) | | Rel. Exp. (%) |
|---|---|---|---|
| | Ag3043, Run | | Ag3043, Run |
| Tissue Name | 167963717 | Tissue Name | 167963717 |
| Liver adenocarcinoma | 79.0 | Kidney (fetal) | 21.6 |
| Pancreas | 4.3 | Renal ca. 786-0 | 19.2 |
| Pancreatic ca. CAPAN 2 | 16.3 | Renal ca. A498 | 18.4 |
| Adrenal gland | 5.9 | Renal ca. RXF 393 | 31.2 |
| Thyroid | 2.8 | Renal ca. ACHN | 19.2 |
| Salivary gland | 2.2 | Renal ca. UO-31 | 46.0 |
| Pituitary gland | 3.3 | Renal ca. TK-10 | 18.2 |
| Brain (fetal) | 7.4 | Liver | 3.5 |
| Brain (whole) | 9.7 | Liver (fetal) | 11.4 |
| Brain (amygdala) | 6.9 | Liver ca. | 18.8 |
| | | (hepatoblast) HepG2 | |
| Brain (cerebellum) | 12.9 | Lung | 3.9 |
| Brain (hippocampus) | 6.6 | Lung (fetal) | 13.0 |
| Brain (substantia nigra) | 8.2 | Lung ca. (small cell) | 19.5 |
| | | LX-1 | |
| Brain (thalamus) | 4.6 | Lung ca. (small cell) | 11.0 |
| | | NCI-H69 | |
| Cerebral Cortex | 10.5 | Lung ca. (s.cell var.) | 55.5 |
| | | SHP-77 | |
| Spinal cord | 5.7 | Lung ca. (large | 3.3 |
| | | cell)NCI-H460 | |
| glio/astro U87-MG | 40.1 | Lung ca. (non-sm. | 25.2 |
| | | cell) A549 | |
| glio/astro U-118-MG | 50.3 | Lung ca. (non-s.cell) | 15.0 |
| | | NCI-H23 | |
| astrocytoma SW1783 | 29.1 | Lung ca. (non-s.cell) | 13.2 |
| | | HOP-62 | |
| neuro*; met SK-N-AS | 11.7 | Lung ca. (non-s.cl) | 12.0 |
| | | NCI-H522 | |
| astrocytoma SF-539 | 23.0 | Lung ca. (squam.) | 27.9 |
| | | SW 900 | |
| astrocytoma SNB-75 | 44.4 | Lung ca. (squam.) | 22.4 |
| | | NCI-H596 | |
| glioma SNB-19 | 15.9 | Mammary gland | 4.6 |
| glioma U251 | 17.3 | Breast ca.* (pl.ef) | 23.3 |
| | | MCF-7 | |
| glioma SF-295 | 28.9 | Breast ca.* (pl.ef) | 46.7 |
| | | MDA-MB-231 | |
| Heart (fetal) | 20.7 | Breast ca.* (pl.ef) | 21.5 |
| | | T47D | |
| Heart | 8.2 | Breast ca. BT-549 | 15.2 |
| Skeletal muscle (fetal) | 29.3 | Breast ca. MDA-N | 42.9 |
| Skeletal muscle | 37.4 | Ovary | 6.1 |
| Bone marrow | 4.9 | Ovarian ca. OVCAR-3 | 10.4 |
| Thymus | 11.0 | Ovarian ca. OVCAR-4 | 19.5 |
| Spleen | 7.0 | Ovarian ca. OVCAR-5 | 39.5 |
| Lymph node | 19.3 | Ovarian ca. OVCAR-8 | 6.3 |
| Colorectal | 12.9 | Ovarian ca. IGROV-1 | 6.5 |
| Stomach | 3.7 | Ovarian ca.* (ascites) | 100.0 |
| | | SK-OV-3 | |
| Small intestine | 5.4 | Uterus | 5.8 |
| Colon ca. SW480 | 14.2 | Placenta | 3.8 |
| Colon ca.* | 45.4 | Prostate | 2.9 |
| SW620(SW480 met) | | | |
| Colon ca. HT29 | 12.7 | Prostate ca.* (bone | 40.9 |
| | | met)PC-3 | |
| Colon ca. HCT-116 | 25.9 | Testis | 2.8 |
| Colon ca. CaCo-2 | 25.2 | Melanoma | 6.8 |
| | | Hs688(A).T | |
| Colon ca. | 15.4 | Melanoma* (met) | 7.7 |
| tissue(ODO3866) | | Hs688(B).T | |
| Colon ca. HCC-2998 | 13.3 | Melanoma UACC-62 | 17.3 |
| Gastric ca.* (liver met) | 10.4 | Melanoma M14 | 11.0 |
| NCI-N87 | | | |
| Bladder | 13.8 | Melanoma LOX | 43.5 |
| | | IMVI | |
| Trachea | 3.0 | Melanoma* (met) | 27.5 |
| | | SK-MEL-5 | |
| Kidney | 9.5 | Adipose | 7.3 |

TABLE BGD
Panel 4D

| | Rel. Exp. (%) | | Rel. Exp. (%) |
|---|---|---|---|
| | Ag3043, Run | | Ag3043, Run |
| Tissue Name | 164315037 | Tissue Name | 164315037 |
| Secondary Th1 act | 38.4 | HUVEC IL-1beta | 20.6 |
| Secondary Th2 act | 38.7 | HUVEC IFN gamma | 22.5 |
| Secondary Tr1 act | 40.3 | HUVEC TNF alpha + IFN | 28.7 |
| | | gamma | |
| Secondary Th1 rest | 10.2 | HUVEC TNF alpha + IL4 | 40.6 |
| Secondary Th2 rest | 18.2 | HUVEC IL-11 | 18.4 |
| Secondary Tr1 rest | 16.3 | Lung Microvascular EC | 23.3 |
| | | none | |
| Primary Th1 act | 47.6 | Lung Microvascular EC | 26.1 |
| | | TNF alpha + IL-1beta | |
| Primary Th2 act | 34.6 | Microvascular Dermal EC | 47.0 |
| | | none | |
| Primary Tr1 act | 46.3 | Microsvasular Dermal EC | 28.5 |
| | | TNF alpha + IL-1beta | |
| Primary Th1 rest | 55.9 | Bronchial epithelium | 54.7 |
| | | TNF alpha + IL1beta | |
| Primary Th2 rest | 28.5 | Small airway epithelium | 29.7 |
| | | none | |
| Primary Tr1 rest | 33.0 | Small airway epithelium | 89.5 |
| | | TNF alpha + IL-1beta | |
| CD45RA CD4 | 31.4 | Coronery artery SMC rest | 33.0 |
| lymphocyte act | | | |
| CD45RO CD4 | 39.8 | Coronery artery SMC | 14.2 |
| lymphocyte act | | TNF alpha + IL-1beta | |
| CD8 lymphocyte act | 47.0 | Astrocytes rest | 10.6 |
| Secondary CD8 | 37.6 | Astrocytes TNF alpha + | 8.2 |
| lymphocyte rest | | IL-1beta | |
| Secondary CD8 | 25.3 | KU-812 (Basophil) rest | 20.4 |
| lymphocyte act | | | |
| CD4 lymphocyte none | 9.5 | KU-812 (Basophil) | 49.7 |
| | | PMA/ionomycin | |
| 2ry Th1/Th2/Tr1_anti- | 21.2 | CCD1106 (Keratinocytes) | 47.3 |
| CD95 CH11 | | none | |
| LAK cells rest | 43.8 | CCD1106 (Keratinocytes) | 25.3 |
| | | TNF alpha + IL-1beta | |
| LAK cells IL-2 | 39.0 | Liver cirrhosis | 4.8 |
| LAK cells IL-2 + IL-12 | 38.4 | Lupus kidney | 5.0 |
| LAK cells IL-2 + IFN | 52.5 | NCI-H292 none | 46.7 |
| gamma | | | |
| LAK cells IL-2 + IL-18 | 42.9 | NCI-H292 IL-4 | 62.9 |
| LAK cells | 15.6 | NCI-H292 IL-9 | 70.7 |
| PMA/ionomycin | | | |
| NK Cells IL-2 rest | 29.3 | NCI-H292 IL-13 | 33.9 |
| Two Way MLR 3 day | 29.3 | NCI-H292 IFN gamma | 35.6 |
| Two Way MLR 5 day | 23.5 | HPAEC none | 17.3 |
| Two Way MLR 7 day | 16.2 | HPAEC TNF alpha + IL-1 | 29.5 |
| | | beta | |
| PBMC rest | 10.9 | Lung fibroblast none | 20.2 |
| PBMC PWM | 91.4 | Lung fibroblast TNF | 17.8 |
| | | alpha + IL-1beta | |
| PBMC PHA-L | 43.5 | Lung fibroblast IL-4 | 40.9 |
| Ramos (B cell) none | 50.3 | Lung fibroblast IL-9 | 40.6 |
| Ramos (B cell) | 100.0 | Lung fibroblast IL-13 | 20.9 |
| ionomycin | | | |
| B lymphocytes PWM | 94.6 | Lung fibroblast IFN | 41.8 |
| | | gamma | |
| B lymphocytes CD40L | 39.0 | Dermal fibroblast | 56.3 |
| and IL-4 | | CCD1070 rest | |
| EOL-1 dbcAMP | 24.7 | Dermal fibroblast | 84.7 |
| | | CCD1070 TNF alpha | |
| EOL-1 dbcAMP | 40.9 | Dermal fibroblast | 25.0 |
| PMA/ionomycin | | CCD1070 IL-1beta | |
| Dendritic cells none | 34.2 | Dermal fibroblast IFN | 29.3 |
| | | gamma | |
| Dendritic cells LPS | 38.7 | Dermal fibroblast IL-4 | 42.6 |
| Dendritic cells anti- | 48.0 | IBD Colitis 2 | 2.0 |
| CD40 | | | |
| Monocytes rest | 17.9 | IBD Crohn's | 1.8 |
| Monocytes LPS | 19.1 | Colon | 17.3 |
| Macrophages rest | 40.6 | Lung | 16.8 |
| Macrophages LPS | 18.4 | Thymus | 17.7 |
| HUVEC none | 37.4 | Kidney | 29.1 |
| HUVEC starved | 65.1 | | |

CNS_neurodegeneration_v1.0 Summary: Ag3043 There is an association with a statistical confidence of 0.1 between increased expression of the NOV64 gene in the temporal cortex and Alzheimer's disease. This gene encodes a homolog of dipeptidyl peptidase, which belongs to a known class of markers of T cell activation in Multiple Sclerosis. This indicates that inhibitors of this gene product may have utility in treatment of this disease. A dipeptidyl peptidase is also dysregulated in Huntington's disease. Our finding of increased expression of this gene product in the temporal cortex of Alzheimer's disease patients indicates that there may be a wider utility of inhibitors of the protein encoded by this gene, including the treatment of neurodegenerative diseases such as Huntington's and Alzheimer's, as well as Multiple Sclerosis.

References:

Khoury S J, Guttmann C R, Orav E J, Kikinis R, Jolesz F A, Weiner H L. Changes in activated T cells in the blood correlate with disease activity in multiple sclerosis. Arch Neurol August 2000;57(8):1183-9

OBJECTIVE: To determine whether changes in activation markers on peripheral blood T cells correlate with disease activity in patients with multiple sclerosis. DESIGN: In a prospective longitudinal study during 1 year, we analyzed the change in percentage of activated T lymphocytes in the peripheral blood of 40 patients with multiple sclerosis in relation to clinical findings and changes on brain magnetic resonance imaging (MRI) scans. The patients underwent repeated imaging of the brain (mean number of MRIs for each patient, 22) at the time blood samples were obtained as well as at monthly neurological examinations, and at the time of scoring on the Kurtzke Expanded Disability Status Scale (EDSS) and ambulation index scale. RESULTS: A change in the percentage of cells expressing the activation markers interleukin 2 receptor (CD25), class II major histocompatibility complex (MHC) (13) or surface dipeptidyl peptidase (CD26) correlated significantly with a change in lesion volume or a change in number of gadolinium-enhancing lesions as detected on MRI. Changes in CD25(+) cells and in CD4(+) cells expressing class II MHC also correlated with changes in disability as measured by EDSS in patients with relapsing-remitting disease, and changes in CD4(+)CD25(+) cells correlated with the occurrence of attacks in patients with relapsing-remitting disease. These correlations are dependent on measurement of changes between time points sampled at 1- or 2-week intervals. CONCLUSION: There is a linkage between peripheral T-lymphocyte activation as measured by cell surface markers and disease activity in patients with multiple sclerosis.

Mantle D, Falkous G, Ishiura S, Perry R H, Perry E K. Comparison of cathepsin protease activities in brain tissue from normal cases and cases with Alzheimer's disease, Lewy body dementia, Parkinson's disease and Huntington's disease. J Neurol Sci July 1995;131(1):65-70

Recent evidence, based upon immunocytochemical and histochemical analysis of brain cortical tissue from alzheimer's disease patients, has suggested that altered activity and/or distribution of the lysosomal proteases cathepsins B and D may be implicated in the abnormal protein processing pathway resulting in formation of the neurotoxic amyloid A4 peptide, characteristic of this neurodegenerative disorder. We have therefore compared, via biochemical assay techniques using conventional or specially synthesised (corresponding to protein cleavage points of relevant to A4 peptide formation) fluorogenic substrates, the levels of activity of the lysosomal proteases cathepsins B, D, H and L, and dipeptidyl aminopeptidases I and II in frontal cortex (grey/white matter) from control and Alzheimer's disease patients. For comparative purposes, activity levels of the above enzymes were also determined in frontal cortex tissue from cases with Lewy body dementia and Parkinson's disease, and in caudate tissue from control and Huntington's disease cases. There was no significant difference in activity for any protease types in tissue from control cases and cases with Alzheimer's disease, Lewy body dementia or Parkinson's disease, with the exception of reduced dipeptidyl aminopeptidase II activity in Lewy body dementia and Parkinson's cases. We have therefore been unable to confirm a potential role for lysosomal cathepsins in the characteristic neurodegeneration associated with Alzheimer's disease; however the finding of significant increases in activity of dipeptidyl aminopeptidase II, cathepsin H and cathepsin D specifically in cases with Huntington's disease is of particular note. We therefore suggest the potential role of the latter enzymes in the pathogenesis of Huntington's disease requires further investigation

Panel 1.3D Summary: Ag3043 Highest expression of the NOV64 gene is seen in an ovarian cancer cell line (CT=26.2). This gene is expressed at moderate levels in all the cancer cell lines in this panel. Thus, this is a potential target for small molecule inhibitor drugs in cancer.

This gene also has moderate levels of expression in pancreas, adrenal, thyroid, pituitary, heart, skeletal muscle, liver and adipose. Therefore, this gene product may be a small molecule target for the treatment of metabolic and endocrine diseases, including obesity and Types 1 and 2 diabetes.

In addition, this gene is expressed in the central nervous system. Please see CNS_neurodegeneration_v1.0 for discussion of utility of this gene in the central nervous system.

Panel 4D Summary: Ag3043 The NOV64 gene is expressed in a number of cells and tissues of immunological importance, especially in activated B cells, T cells, dendritic cells, and activated lung and skin fibroblasts. Therefore, small molecule antagonists that block the function of the NOV64 gene product may reduce or eliminate the symptoms of a wide range of autoimmune and inflammatory diseases, including Crohn's disease, ulcerative colitis, multiple sclerosis, chronic obstructive pulmonary disease, asthma, emphysema, rheumatoid arthritis, lupus erythematosus, or psoriasis.

NOV65a and NOV65b

Expression of gene NOV65a and variant NOV65b was assessed using the primer-probe sets Ag3020 and Ag2968, described in Tables BHA and BHB. Results of the RTQ-PCR runs are shown in Tables BHC, BHD, BHE, BHF and BHG.

TABLE BHA
Probe Name Ag3020

| | | | Start | |
|---|---|---|---|---|
| Primers | Sequences | Length | Position | SEQ ID NO: |
| Forward | 5′-ggaatcacccacattctgaat-3′ | 21 | 193 | 1261 |
| Probe | TET-5′-cgtttacactggccccgaattctaca-3′-TAMRA | 26 | 231 | 1262 |
| Reverse | 5′-cctctacacccaggtactggat-3′ | 22 | 268 | 1263 |

TABLE BHB
Probe Name Ag2968

| | | | Start | |
|---|---|---|---|---|
| Primers | Sequences | Length | Position | SEQ ID NO: |
| Forward | 5′-ggaatcacccacattctgaat-3′ | 21 | 193 | 1264 |
| Probe | TET-5′-cgtttacactggccccgaattctaca-3′-TAMRA | 26 | 231 | 1265 |
| Reverse | 5′-cctctacacccaggtactggat-3′ | 22 | 268 | 1266 |

TABLE BHC
General_screening_panel_v1.4

| | Rel. Exp. (%) | | Rel. Exp. (%) |
|---|---|---|---|
| | Ag3020, Run | | Ag3020, Run |
| Tissue Name | 221998694 | Tissue Name | 221998694 |
| Adipose | 0.4 | Renal ca. TK-10 | 0.0 |
| Melanoma* | 0.0 | Bladder | 0.1 |
| Hs688(A).T | | | |
| Melanoma* | 0.0 | Gastric ca. (liver met.) | 0.0 |
| Hs688(B).T | | NCI-N87 | |
| Melanoma* M14 | 0.0 | Gastric ca. KATO III | 0.0 |
| Melanoma* | 0.0 | Colon ca. SW-948 | 0.0 |
| LOXIMVI | | | |
| Melanoma* SK- | 0.0 | Colon ca. SW480 | 0.2 |
| MEL-5 | | | |
| Squamous cell | 0.0 | Colon ca.* (SW480 | 0.0 |
| carcinoma SCC-4 | | met) SW620 | |
| Testis Pool | 0.0 | Colon ca. HT29 | 0.0 |
| Prostate ca.* (bone | 0.0 | Colon ca. HCT-116 | 0.0 |
| met) PC-3 | | | |
| Prostate Pool | 0.2 | Colon ca. CaCo-2 | 0.0 |
| Placenta | 0.1 | Colon cancer tissue | 31.0 |
| Uterus Pool | 0.0 | Colon ca. SW1116 | 0.0 |
| Ovarian ca. | 0.0 | Colon ca. Colo-205 | 100.0 |
| OVCAR-3 | | | |
| Ovarian ca. SK-OV-3 | 0.3 | Colon ca. SW-48 | 5.7 |
| Ovarian ca. | 0.0 | Colon Pool | 0.0 |
| OVCAR-4 | | | |
| Ovarian ca. | 0.1 | Small Intestine Pool | 0.0 |
| OVCAR-5 | | | |
| Ovarian ca. IGROV-1 | 18.9 | Stomach Pool | 0.0 |
| Ovarian ca. | 0.0 | Bone Marrow Pool | 0.0 |
| OVCAR-8 | | | |
| Ovary | 0.1 | Fetal Heart | 12.7 |
| Breast ca. MCF-7 | 0.0 | Heart Pool | 4.9 |
| Breast ca. MDA- | 0.0 | Lymph Node Pool | 0.1 |
| MB-231 | | | |
| Breast ca. BT 549 | 0.0 | Fetal Skeletal Muscle | 23.3 |
| Breast ca. T47D | 0.0 | Skeletal Muscle Pool | 20.0 |
| Breast ca. MDA-N | 0.0 | Spleen Pool | 0.0 |
| Breast Pool | 0.0 | Thymus Pool | 0.0 |
| Trachea | 0.3 | CNS cancer (glio/astro) | 0.0 |
| | | U87-MG | |
| Lung | 0.0 | CNS cancer (glio/astro) | 0.1 |
| | | U-118-MG | |
| Fetal Lung | 0.2 | CNS cancer | 0.0 |
| | | (neuro;met) SK-N-AS | |
| Lung ca. NCI-N417 | 0.0 | CNS cancer (astro) SF- | 0.7 |
| | | 539 | |
| Lung ca. LX-1 | 12.7 | CNS cancer (astro) | 0.1 |
| | | SNB-75 | |
| Lung ca. NCI-H146 | 0.1 | CNS cancer (glio) | 15.9 |
| | | SNB-19 | |
| Lung ca. SHP-77 | 0.0 | CNS cancer (glio) SF- | 0.5 |
| | | 295 | |
| Lung ca. A549 | 0.0 | Brain (Amygdala) Pool | 0.1 |
| Lung ca. NCI-H526 | 0.0 | Brain (cerebellum) | 0.0 |
| Lung ca. NCI-H23 | 0.0 | Brain (fetal) | 0.1 |
| Lung ca. NCI-H460 | 0.0 | Brain (Hippocampus) | 0.1 |
| | | Pool | |
| Lung ca. HOP-62 | 0.1 | Cerebral Cortex Pool | 0.1 |
| Lung ca. NCI-H522 | 0.0 | Brain (Substantia nigra) | 0.1 |
| | | Pool | |
| Liver | 0.0 | Brain (Thalamus) Pool | 0.1 |
| Fetal Liver | 0.1 | Brain (whole) | 0.1 |
| Liver ca. HepG2 | 0.0 | Spinal Cord Pool | 0.0 |
| Kidney Pool | 0.1 | Adrenal Gland | 0.0 |
| Fetal Kidney | 0.1 | Pituitary gland Pool | 0.0 |
| Renal ca. 786-0 | 0.0 | Salivary Gland | 0.1 |
| Renal ca. A498 | 0.0 | Thyroid (female) | 0.0 |
| Renal ca. ACHN | 0.0 | Pancreatic ca. CAPAN2 | 0.4 |
| Renal ca. UO-31 | 0.0 | Pancreas Pool | 0.0 |

TABLE BHD
Panel 1.3D

| | Rel. Exp.(%) | Rel. Exp.(%) |
|---|---|---|
| | Ag2968, Run | Ag3020, Run |
| Tissue Name | 166220058 | 167819114 |
| Liver | 0.0 | 0.1 |
| adenocarcinoma | | |
| Pancreas | 0.0 | 0.0 |
| Pancreatic ca. | 0.1 | 0.2 |
| CAPAN2 | | |
| Adrenal gland | 0.0 | 0.0 |
| Thyroid | 0.3 | 1.6 |
| Salivary gland | 0.8 | 0.6 |
| Pituitary gland | 0.0 | 0.0 |
| Brain (fetal) | 0.0 | 0.1 |
| Brain (whole) | 0.4 | 1.0 |
| Brain (amygdala) | 0.2 | 1.0 |
| Brain (cerebellum) | 0.0 | 0.0 |
| Brain | 0.0 | 0.0 |
| (hippocampus) | | |
| Brain (substantia | 0.2 | 0.1 |
| nigra) | | |
| Brain (thalamus) | 0.0 | 0.0 |
| Cerebral Cortex | 0.4 | 0.1 |
| Spinal Cord | 0.0 | 0.0 |
| glio/astro U87-MG | 0.0 | 0.0 |
| glio/astro U-118- | 0.0 | 0.1 |
| MG | | |
| astrocytoma | 0.0 | 0.0 |
| SW1783 | | |
| neuro*; met SK-N- | 0.0 | 0.0 |
| AS | | |
| astrocytoma SF- | 12.1 | 8.4 |
| 539 | | |
| astrocytoma SNB- | 0.0 | 0.3 |
| 75 | | |
| glioma SNB-19 | 0.0 | 0.0 |
| glioma U251 | 0.4 | 0.0 |
| glioma SF-295 | 0.3 | 0.3 |
| Heart (fetal) | 7.4 | 26.8 |
| Heart | 29.9 | 35.4 |
| Skeletal muscle | 10.8 | 33.9 |
| (fetal) | | |
| Skeletal muscle | 100.0 | 100.0 |
| Bone Marrow | 0.1 | 0.6 |
| Thymus | 0.1 | 0.1 |
| Spleen | 0.0 | 0.0 |
| Lymph node | 0.0 | 0.0 |
| Colorectal | 0.2 | 0.2 |
| Stomach | 0.0 | 0.0 |
| Small intestine | 0.0 | 0.0 |
| Colon ca. SW480 | 0.0 | 0.2 |
| Colon ca.* | 1.6 | 6.1 |
| SW620(SW480 | | |
| met) | | |
| Colon ca. HT29 | 0.0 | 0.0 |
| Colon ca. HCT- | 0.0 | 0.0 |
| 116 | | |
| Colon ca. CaCo-2 | 0.0 | 0.0 |
| Colon ca. | 21.9 | 30.6 |
| tissue(ODO3866) | | |
| Colon ca. HCC- | 0.0 | 0.5 |
| 2998 | | |
| Gastric ca.* (liver | 0.0 | 0.0 |
| met) NCI-N87 | | |
| Bladder | 0.1 | 0.0 |
| Trachea | 0.8 | 0.6 |
| Kidney | 0.0 | 0.0 |
| Kidney(fetal) | 0.0 | 0.3 |
| Renal ca. 786- | 0.0 | 0.0 |
| 0 | | |
| Renal ca. | 0.0 | 0.0 |
| A498 | | |
| Renal ca. RXF | 0.0 | 0.0 |
| 393 | | |
| Renal ca. | 0.0 | 0.0 |
| ACHN | | |
| Renal ca. UO- | 0.0 | 0.0 |
| 31 | | |
| Renal ca. TK- | 0.0 | 0.0 |
| 10 | | |
| Liver | 0.0 | 0.0 |
| Liver (fetal) | 0.5 | 0.0 |
| Liver ca. | 0.0 | 0.0 |
| (hepatoblast) | | |
| HepG2 | | |
| Lung | 0.0 | 0.5 |
| Lung (fetal) | 0.0 | 0.0 |
| Lung ca. | 10.7 | 16.2 |
| (small cell) | | |
| LX-1 | | |
| Lung ca. | 0.0 | 0.5 |
| (small cell) | | |
| NCI-H69 | | |
| Lung ca. | 0.0 | 0.0 |
| (s.cell var.) | | |
| SHP-77 | | |
| Lung ca. (large | 0.0 | 0.0 |
| cell)NCI-H460 | | |
| Lung ca. (non- | 0.0 | 0.2 |
| sm. cell) A549 | | |
| Lung ca. (non- | 0.0 | 0.0 |
| s.cell) NCI- | | |
| H23 | | |
| Lung ca. (non- | 0.1 | 0.1 |
| s.cell) HOP-62 | | |
| Lung ca. (non- | 0.0 | 0.0 |
| s.cl) NCI- | | |
| H522 | | |
| Lung ca. | 0.0 | 0.0 |
| (squam.) SW | | |
| 900 | | |
| Lung ca. | 0.1 | 0.4 |
| (squam.) NCI- | | |
| H596 | | |
| Mammary | 0.2 | 0.2 |
| gland | | |
| Breast ca.* | 0.0 | 0.0 |
| (pl.ef) MCF-7 | | |
| Breast ca.* | 0.0 | 0.0 |
| (pl.ef) MDA- | | |
| 1267 | | |
| MB-231 | | |
| Breast ca.* | 0.0 | 0.0 |
| (pl.ef) T47D | | |
| Breast ca. BT- | 0.0 | 0.0 |
| 549 | | |
| Breast ca. | 0.0 | 0.0 |
| MDA-N | | |
| Ovary | 0.0 | 0.1 |
| Ovarian ca. | 0.0 | 0.0 |
| OVCAR-3 | | |
| Ovarian ca. | 0.0 | 0.0 |
| OVCAR-4 | | |
| Ovarian ca. | 0.0 | 0.6 |
| OVCAR-5 | | |
| Ovarian ca. | 0.0 | 0.0 |
| OVCAR-8 | | |
| Ovarian ca. | 26.2 | 26.2 |
| IGROV-1 | | |
| Ovarian ca.* | 0.2 | 1.0 |
| (ascites) SK- | | |
| OV-3 | | |
| Uterus | 0.0 | 0.0 |
| Placenta | 1.0 | 0.0 |
| Prostate | 0.2 | 0.1 |
| Prostate ca.* | 0.0 | 0.0 |
| (bone met)PC- | | |
| 3 | | |
| Testis | 0.2 | 0.2 |
| Melanoma | 0.0 | 0.0 |
| Hs688(A).T | | |
| Melanoma* | 0.0 | 0.0 |
| (met) | | |
| Hs688(B).T | | |
| Melanoma | 0.0 | 0.0 |
| UACC-62 | | |
| Melanoma | 0.0 | 0.0 |
| M14 | | |
| Melanoma | 0.0 | 0.0 |
| LOX IMVI | | |
| Melanoma* | 0.0 | 0.0 |
| (met) SK- | | |
| MEL-5 | | |
| Adipose | 1.1 | 1.7 |

TABLE BHE
Panel 3D

| | Rel. Exp. (%) | | Rel. Exp. (%) |
|---|---|---|---|
| | Ag2968, Run | | Ag2968, Run |
| Tissue Name | 170188142 | Tissue Name | 170188142 |
| Daoy-Medulloblastoma | 0.0 | Ca Ski-Cervical epidermoid | 0.0 |
| | | carcinoma (metastasis) | |
| TE671-Medulloblastoma | 2.2 | ES-2-Ovarian clear cell | 0.0 |
| | | carcinoma | |
| D283 Med- | 0.0 | Ramos-Stimulated with | 0.0 |
| Medulloblastoma | | PMA/ionomycin 6 h | |
| PFSK-1-Primitive | 0.0 | Ramos-Stimulated with | 0.0 |
| Neuroectodermal | | PMA/ionomycin 14 h | |
| XF-498-CNS | 0.0 | MEG-01-Chronic | 0.0 |
| | | myelogenous leukemia | |
| | | (megokaryoblast) | |
| SNB-78-Glioma | 0.0 | Raji-Burkitt's lymphoma | 0.0 |
| SF-268-Glioblastoma | 0.0 | Daudi-Burkitt's lymphoma | 0.0 |
| T98G-Glioblastoma | 0.0 | U266-B-cell plasmacytoma | 0.0 |
| SK-N-SH- | 0.0 | CA46-Burkitt's lymphoma | 0.0 |
| Neuroblastoma | | | |
| (metastasis) | | | |
| SF-295-Glioblastoma | 0.0 | RL-non-Hodgkin's B-cell | 0.0 |
| | | lymphoma | |
| Cerebellum | 0.0 | JM1-pre-B-cell lymphoma | 0.0 |
| Cerebellum | 0.0 | Jurkat-T cell leukemia | 0.0 |
| NCI-H292- | 0.0 | TF-1-Erythroleukemia | 0.0 |
| Mucoepidermoid lung | | | |
| carcinoma | | | |
| DMS-114-Small cell | 0.0 | HUT 78-T-cell lymphoma | 0.0 |
| lung cancer | | | |
| DMS-79-Small cell lung | 0.0 | U937-Histiocytic lymphoma | 0.0 |
| cancer | | | |
| NCI-H146-Small cell | 0.0 | KU-812-Myelogenous | 0.0 |
| lung cancer | | leukemia | |
| NCI-H526-Small cell | 0.0 | 769-P-Clear cell renal | 0.0 |
| lung cancer | | carcinoma | |
| NCI-N417-Small cell | 0.0 | Caki-2-Clear cell renal | 0.0 |
| lung cancer | | carcinoma | |
| NCI-H82-Small cell | 0.0 | SW 839-Clear cell renal | 0.0 |
| lung cancer | | carcinoma | |
| NCI-H157-Squamous | 0.0 | G401-Wilms' tumor | 0.0 |
| cell lung cancer | | | |
| (metastasis) | | | |
| NCI-H1155-Large cell | 0.0 | Hs766T-Pancreatic | 0.0 |
| lung cancer | | carcinoma (LN metastasis) | |
| NCI-H1299-Large cell | 0.0 | CAPAN-1-Pancreatic | 0.0 |
| lung cancer | | adenocarcinoma (liver | |
| | | metastasis) | |
| NCI-H727-Lung | 0.0 | SU86.86-Pancreatic | 0.0 |
| carcinoid | | carcinoma (liver metastasis) | |
| NCI-UMC-11-Lung | 0.0 | BxPC-3-Pancreatic | 0.0 |
| carcinoid | | adenocarcinoma | |
| LX-1-Small cell lung | 4.4 | HPAC-Pancreatic | 0.0 |
| cancer | | adenocarcinoma | |
| Colo-205-Colon cancer | 100.0 | MIA PaCa-2-Pancreatic | 0.0 |
| | | carcinoma | |
| KM12-Colon cancer | 0.0 | CFPAC-1-Pancreatic ductal | 0.0 |
| | | adenocarcinoma | |
| KM20L2-Colon cancer | 0.0 | PANC-1-Pancreatic | 0.0 |
| | | epithelioid ductal carcinoma | |
| NCI-H716-Colon cancer | 0.0 | T24-Bladder carcinma | 0.0 |
| | | (transitional cell) | |
| SW-48-Colon | 4.3 | 5637-Bladder carcinoma | 0.0 |
| adenocarcinoma | | | |
| SW1116-Colon | 0.0 | HT-1197-Bladder carcinoma | 0.0 |
| adenocarcinoma | | | |
| LS 174T-Colon | 10.1 | UM-UC-3-Bladder carcinma | 0.0 |
| adenocarcinoma | | (transitional cell) | |
| SW-948-Colon | 0.0 | A204-Rhabdomyosarcoma | 0.0 |
| adenocarcinoma | | | |
| SW-480-Colon | 0.0 | HT-1080-Fibrosarcoma | 0.0 |
| adenocarcinoma | | | |
| NCI-SNU-5-Gastric | 0.0 | MG-63-Osteosarcoma | 0.0 |
| carcinoma | | | |
| KATO III-Gastric | 1.2 | SK-LMS-1-Leiomyosarcoma | 0.0 |
| carcinoma | | (vulva) | |
| NCI-SNU-16-Gastric | 0.0 | SJRH30-Rhabdomyosarcoma | 0.7 |
| carcinoma | | (met to bone marrow) | |
| NCI-SNU-1-Gastric | 0.1 | A431-Epidermoid carcinoma | 0.0 |
| carcinoma | | | |
| RF-1-Gastric | 0.0 | WM266-4-Melanoma | 0.0 |
| adenocarcinoma | | | |
| RF-48-Gastric | 0.0 | DU 145-Prostate carcinoma | 0.0 |
| adenocarcinoma | | (brain metastasis) | |
| MKN-45-Gastric | 0.0 | MDA-MB-468-Breast | 0.0 |
| carcinoma | | adenocarcinoma | |
| NCI-N87-Gastric | 0.0 | SCC-4-Squamous cell | 0.0 |
| carcinoma | | carcinoma of tongue | |
| OVCAR-5-Ovarian | 0.0 | SCC-9-Squamous cell | 0.0 |
| carcinoma | | carcinoma of tongue | |
| RL95-2-Uterine | 0.0 | SCC-15-Squamous cell | 0.0 |
| carcinoma | | carcinoma of tongue | |
| HelaS3-Cervical | 0.0 | CAL 27-Squamous cell | 0.0 |
| adenocarcinoma | | carcinoma of tongue | |

TABLE BHF
Panel 4D

| | Rel. Exp. (%) | | Rel. Exp. (%) |
|---|---|---|---|
| | Ag3020, Run | | Ag3020, Run |
| Tissue Name | 164528102 | Tissue Name | 164528102 |
| Secondary Th1 act | 0.0 | HUVEC IL-1beta | 0.0 |
| Secondary Th2 act | 0.0 | HUVEC IFN gamma | 0.0 |
| Secondary Tr1 act | 0.0 | HUVEC TNF alpha + IFN | 0.0 |
| | | gamma | |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC | 0.0 |
| | | none | |
| Primary Th1 act | 0.0 | Lung Microvascular EC | 0.0 |
| | | TNF alpha + IL-1beta | |
| Primary Th2 act | 0.0 | Microvascular Dermal EC | 0.0 |
| | | none | |
| Primary Tr1 act | 0.0 | Microsvasular Dermal EC | 0.0 |
| | | TNF alpha + IL-1beta | |
| Primary Th1 rest | 0.0 | Bronchial epithelium | 0.0 |
| | | TNF alpha + IL1beta | |
| Primary Th2 rest | 0.0 | Small airway epithelium | 0.0 |
| | | none | |
| Primary Tr1 rest | 0.0 | Small airway epithelium | 0.0 |
| | | TNF alpha + IL-1beta | |
| CD45RA CD4 | 0.0 | Coronery artery SMC rest | 0.0 |
| lymphocyte act | | | |
| CD45RO CD4 | 0.0 | Coronery artery SMC | 0.0 |
| lymphocyte act | | TNF alpha + IL-1beta | |
| CD8 lymphocyte act | 0.0 | Astrocytes rest | 65.1 |
| Secondary CD8 | 0.0 | Astrocytes TNF alpha + | 21.0 |
| lymphocyte rest | | IL-1beta | |
| Secondary CD8 | 0.0 | KU-812 (Basophil) rest | 10.7 |
| lymphocyte act | | | |
| CD4 lymphocyte none | 0.0 | KU-812 (Basophil) | 0.0 |
| | | PMA/ionomycin | |
| 2ry Th1/Th2/Tr1_anti- | 0.0 | CCD1106 (Keratinocytes) | 0.0 |
| CD95 CH11 | | none | |
| LAK cells rest | 13.3 | CCD1106 (Keratinocytes) | 0.0 |
| | | TNF alpha + IL-1beta | |
| LAK cells IL-2 | 0.0 | Liver cirrhosis | 100.0 |
| LAK cells IL-2 + IL-12 | 0.0 | Lupus kidney | 0.0 |
| LAK cells IL-2 + IFN | 0.0 | NCI-H292 none | 0.0 |
| gamma | | | |
| LAK cells IL-2 + IL-18 | 0.0 | NCI-H292 IL-4 | 0.0 |
| LAK cells | 0.0 | NCI-H292 IL-9 | 0.0 |
| PMA/ionomycin | | | |
| NK Cells IL-2 rest | 8.0 | NCI-H292 IL-13 | 0.0 |
| Two Way MLR 3 day | 0.0 | NCI-H292 IFN gamma | 0.0 |
| Two Way MLR 5 day | 0.0 | HPAEC none | 0.0 |
| Two Way MLR 7 day | 10.6 | HPAEC TNF alpha + IL-1 | 11.2 |
| | | beta | |
| PBMC rest | 0.0 | Lung fibroblast none | 0.0 |
| PBMC PWM | 0.0 | Lung fibroblast TNF | 0.0 |
| | | alpha + IL-1beta | |
| PBMC PHA-L | 0.0 | Lung fibroblast IL-4 | 0.0 |
| Ramos (B cell) none | 0.0 | Lung fibroblast IL-9 | 0.0 |
| Ramos (B cell) | 0.0 | Lung fibroblast IL-13 | 0.0 |
| ionomycin | | | |
| B lymphocytes PWM | 0.0 | Lung fibroblast IFN | 0.0 |
| | | gamma | |
| B lymphocytes CD40L | 9.9 | Dermal fibroblast | 0.0 |
| and IL-4 | | CCD1070 rest | |
| EOL-1 dbcAMP | 0.0 | Dermal fibroblast | 0.0 |
| | | CCD1070 TNF alpha | |
| EOL-1 dbcAMP | 0.0 | Dermal fibroblast | 0.0 |
| PMA/ionomycin | | CCD1070 IL-1beta | |
| Dendritic cells none | 23.8 | Dermal fibroblast IFN | 0.0 |
| | | gamma | |
| Dendritic cells LPS | 0.0 | Dermal fibroblast IL-4 | 0.0 |
| Dendritic cells anti- | 0.0 | IBD Colitis 2 | 0.0 |
| CD40 | | | |
| Monocytes rest | 0.0 | IBD Crohn's | 0.0 |
| Monocytes LPS | 0.0 | Colon | 44.4 |
| Macrophages rest | 0.0 | Lung | 26.6 |
| Macrophages LPS | 0.0 | Thymus | 0.0 |
| HUVEC none | 0.0 | Kidney | 0.0 |
| HUVEC starved | 0.0 | | |

TABLE BHG
Panel 5D

| | Rel. Exp. (%) | | Rel. Exp. (%) |
|---|---|---|---|
| | Ag3020, Run | | Ag3020, Run |
| Tissue Name | 172171108 | Tissue Name | 172171108 |
| 97457_Patient- | 0.2 | 94709_Donor 2 AM - A_adipose | 0.0 |
| 02go_adipose | | | |
| 97476_Patient- | 8.4 | 94710_Donor 2 AM - B_adipose | 0.0 |
| 07sk_skeletal muscle | | | |
| 97477_Patient- | 0.0 | 94711_Donor 2 AM - C_adipose | 0.0 |
| 07ut_uterus | | | |
| 97478_Patient- | 1.3 | 94712_Donor 2 AD - A_adipose | 3.0 |
| 07pl_placenta | | | |
| 97481_Patient- | 12.6 | 94713_Donor 2 AD - B_adipose | 0.0 |
| 08sk_skeletal muscle | | | |
| 97482_Patient- | 0.0 | 94714_Donor 2 AD - C_adipose | 0.0 |
| 08ut_uterus | | | |
| 97483_Patient- | 0.0 | 94742_Donor 3 U - | 0.0 |
| 08pl_placenta | | A_Mesenchymal Stem Cells | |
| 97486_Patient- | 12.9 | 94743_Donor 3 U — | 0.0 |
| 09sk_skeletal muscle | | B_Mesenchymal Stem Cells | |
| 97487_Patient- | 0.7 | 94730_Donor 3 AM - A_adipose | 0.0 |
| 09ut_uterus | | | |
| 97488_Patient- | 0.2 | 94731_Donor 3 AM - B_adipose | 0.0 |
| 09pl_placenta | | | |
| 97492_Patient- | 0.2 | 94732_Donor 3 AM - C_adipose | 0.0 |
| 10ut_uterus | | | |
| 97493_Patient- | 1.1 | 94733_Donor 3 AD - A_adipose | 0.0 |
| 10pl_placenta | | | |
| 97495_Patient- | 0.0 | 94734_Donor 3 AD - B_adipose | 0.0 |
| 11go_adipose | | | |
| 97496_Patient- | 53.6 | 94735_Donor 3 AD - C_adipose | 0.0 |
| 11sk_skeletal muscle | | | |
| 97497_Patient- | 0.3 | 77138_Liver_HepG2untreated | 0.0 |
| 11ut_uterus | | | |
| 97498_Patient- | 2.3 | 73556_Heart_Cardiac stromal | 0.0 |
| 11pl_placenta | | cells (primary) | |
| 97500_Patient- | 0.4 | 81735_Small Intestine | 0.0 |
| 12go_adipose | | | |
| 97501_Patient- | 100.0 | 72409_Kidney_Proximal | 0.0 |
| 12sk_skeletal muscle | | Convoluted Tubule | |
| 97502_Patient- | 0.0 | 82685_Small intestine_Duodenum | 0.0 |
| 12ut_uterus | | | |
| 97503_Patient- | 1.3 | 90650_Adrenal_Adrenocortical | 0.2 |
| 12pl_placenta | | adenoma | |
| 94721_Donor 2 U - | 0.0 | 72410_Kidney_HRCE | 0.3 |
| A_Mesenchymal Stem | | | |
| Cells | | | |
| 94722_Donor 2 U - | 0.0 | 72411_Kidney_HRE | 0.0 |
| B_Mesenchymal Stem | | | |
| Cells | | | |
| 94723_Donor 2 U - | 0.0 | 73139_Uterus_Uterine smooth | 0.0 |
| C_Mesenchymal Stem | | muscle cells | |
| Cells | | | |

General_screening_panel_v1.4 Summary: Ag3020 The NOV65a gene is expressed in brain, colon, lung and ovarian cancer cell lines with highest expression in a colon cancer cell line Colo-205 (CT=24.37). This suggests that this gene can be used as a diagnostic marker for these types of cancer. Furthermore, inhibition of the protein using small molecule drugs could potentially be useful for the treatment of brain, colon, lung and ovarian cancer.

In addition, this gene has low expression in adipose and high expression in adult and fetal heart and skeletal muscle. Thus, this protein phosphatase may be a small molecule target for the treatment of obesity, Type 2 diabetes and cardiac and skeletal muscle disease.

Panel 1.3D Summary: Ag2968/Ag3020 Results from two experiments using identical probe/primer sets are in excellent agreement. Expression of the NOV65a gene is highest in adult skeletal muscle (CTs=26-28). Significant but somewhat lower expression is also seen in fetal skeletal muscle and adult/fetal heart. Thus, expression of this gene may be used to distinguish these samples from the other samples on this panel.

This gene is also expressed in brain, colon, lung and ovarian cancer cell lines, consistent with General_screening_panel_v1.4. This suggests that this gene can be used as a diagnostic marker for these types of cancer and inhibition of the protein using small molecule drugs can be used for the treatment of brain, colon, lung and ovarian cancer.

Panel 3D Summary: Ag2968 Expression of the NOV65a gene is highest in colon cancer cell line Colo-205 (CT=25.6). In addition, significant expression of this gene is seen in two other colon cancer cell lines. Thus, expression of this gene may be used to distinguish these colon cancer cell lines from the other samples on this panel. Moreover, therapeutic modulation of the activity of this gene or its protein product, using small molecules, antibodies or protein therapeutics, may be of benefit in the treatment of colon cancer.

Panel 4D Summary: Ag3020 Expression of the NOV65a gene is highest in a liver cirrhosis sample (CT=33.3). Furthermore, expression of this gene is not detected in normal liver in Panels 1.3D or 1.4, suggesting that its expression is unique to liver cirrhosis. This gene encodes a putative protein phosphatase; therefore, antibodies or small molecule therapeutics could reduce or inhibit fibrosis that occurs in liver cirrhosis. In addition, antibodies to this protein could also be used for the diagnosis of liver cirrhosis. Low levels of expression are also seen in colon and resting astrocytes.

Ag2968 Expression of this gene is low/undetectable (CTs>35) across all of the samples on this panel (data not shown).

Panel 5D Summary: Ag3020 Expression of the NOV65a gene is primarily restricted to samples from skeletal muscle. This specific expression is in agreement with the results in Panels 1.3D and 1.4. Thus, expression of this gene could be used to differentiate between skeletal muscle and other samples on this panel, and as a marker of skeletal muscle. Results from one experiment with the probe and primer set Ag2968 are not included. The amp plot indicates that there were experimental difficulties with this run.

NOV66

Expression of gene NOV66 was assessed using the primer-probe set Ag2913, described in Table BIA. Results of the RTQ-PCR runs are shown in Tables BIB, BIC and BID.

TABLE BIA
Probe Name Ag2913

| | | | Start | |
|---|---|---|---|---|
| Primers | Sequences | Length | Position | SEQ ID NO: |
| Foward | 5′-tttgtggcttgatggcttt-3′ | 19 | 956 | 1267 |
| Probe | TET-5′-ttcctttccgcatttcctatgtgaat-3′-TAMRA | 26 | 977 | 1268 |
| Reverse | 5′-ttccagttaaaggcataacgaa-3′ | 22 | 1012 | 1269 |

TABLE BIB
Panel 1.3D

| | Rel. Exp. (%) | | Rel. Exp. (%) |
|---|---|---|---|
| | Ag2913, Run | | Ag2913, Run |
| Tissue Name | 157366466 | Tissue Name | 157366466 |
| Liver adenocarcinoma | 1.2 | Kidney (fetal) | 4.3 |
| Pancreas | 0.4 | Renal ca. 786-0 | 0.6 |
| Pancreatic ca. CAPAN 2 | 0.0 | Renal ca. A498 | 0.6 |
| Adrenal gland | 2.3 | Renal ca. RXF 393 | 0.2 |
| Thyroid | 0.7 | Renal ca. ACHN | 0.0 |
| Salivary gland | 8.3 | Renal ca. UO-31 | 0.0 |
| Pituitary gland | 0.3 | Renal ca. TK-10 | 0.0 |
| Brain (fetal) | 26.4 | Liver | 2.0 |
| Brain (whole) | 6.4 | Liver (fetal) | 3.5 |
| Brain (amygdala) | 18.4 | Liver ca. | 0.0 |
| | | (hepatoblast) HepG2 | |
| Brain (cerebellum) | 8.6 | Lung | 9.2 |
| Brain (hippocampus) | 100.0 | Lung (fetal) | 4.7 |
| Brain (substantia nigra) | 2.6 | Lung ca. (small cell) | 0.6 |
| | | LX-1 | |
| Brain (thalamus) | 9.5 | Lung ca. (small cell) | 2.1 |
| | | NCI-H69 | |
| Cerebral Cortex | 31.2 | Lung ca. (s.cell var.) | 3.8 |
| | | SHP-77 | |
| Spinal cord | 1.0 | Lung ca. (large | 1.5 |
| | | cell)NCI-H460 | |
| glio/astro U87-MG | 1.2 | Lung ca. (non-sm. | 2.2 |
| | | cell) A549 | |
| glio/astro U-118-MG | 18.9 | Lung ca. (non-s.cell) | 7.2 |
| | | NCI-H23 | |
| astrocytoma SW1783 | 2.8 | Lung ca. (non-s.cell) | 0.0 |
| | | HOP-62 | |
| neuro*; met SK-N-AS | 8.2 | Lung ca. (non-s.cl) | 0.0 |
| | | NCI-H522 | |
| astrocytoma SF-539 | 2.5 | Lung ca. (squam.) | 0.9 |
| | | SW 900 | |
| astrocytoma SNB-75 | 0.7 | Lung ca. (squam.) | 0.8 |
| | | NCI-H596 | |
| glioma SNB-19 | 1.6 | Mammary gland | 5.7 |
| glioma U251 | 0.4 | Breast ca.* (pl.ef) | 2.2 |
| | | MCF-7 | |
| glioma SF-295 | 0.0 | Breast ca.* (pl.ef) | 3.1 |
| | | MDA-MB-231 | |
| Heart (fetal) | 0.1 | Breast ca.* (pl.ef) | 1.4 |
| | | T47D | |
| Heart | 0.4 | Breast ca. BT-549 | 2.6 |
| Skeletal muscle (fetal) | 0.4 | Breast ca. MDA-N | 1.8 |
| Skeletal muscle | 0.5 | Ovary | 0.0 |
| Bone marrow | 8.5 | Ovarian ca. OVCAR-3 | 0.9 |
| Thymus | 2.6 | Ovarian ca. OVCAR-4 | 0.0 |
| Spleen | 7.7 | Ovarian ca. OVCAR-5 | 0.0 |
| Lymph node | 9.1 | Ovarian ca. OVCAR-8 | 1.0 |
| Colorectal | 8.4 | Ovarian ca. IGROV-1 | 0.0 |
| Stomach | 0.9 | Ovarian ca.* (ascites) | 0.9 |
| | | SK-OV-3 | |
| Small intestine | 11.2 | Uterus | 1.2 |
| Colon ca. SW480 | 0.0 | Placenta | 1.7 |
| Colon ca.* | 0.0 | Prostate | 1.9 |
| SW620(SW480 met) | | | |
| Colon ca. HT29 | 0.0 | Prostate ca.* (bone | 0.0 |
| | | met)PC-3 | |
| Colon ca. HCT-116 | 0.1 | Testis | 2.8 |
| Colon ca. CaCo-2 | 7.7 | Melanoma | 0.2 |
| | | Hs688(A).T | |
| Colon ca. | 1.1 | Melanoma* (met) | 0.8 |
| tissue(ODO3866) | | Hs688(B).T | |
| Colon ca. HCC-2998 | 7.9 | Melanoma UACC-62 | 0.0 |
| Gastric ca.* (liver met) | 9.2 | Melanoma M14 | 0.7 |
| NCI-N87 | | | |
| Bladder | 6.4 | Melanoma LOX | 1.5 |
| | | IMVI | |
| Trachea | 1.4 | Melanoma* (met) | 1.3 |
| | | SK-MEL-5 | |
| Kidney | 1.4 | Adipose | 2.3 |

TABLE BIC
Panel 2D

| | Rel. Exp. (%) | | Rel. Exp. (%) |
|---|---|---|---|
| | Ag2913, Run | | Ag2913, Run |
| Tissue Name | 157366467 | Tissue Name | 157366467 |
| Normal Colon | 32.5 | Kidney Margin | 0.0 |
| | | 8120608 | |
| CC Well to Mod Diff | 11.2 | Kidney Cancer | 0.0 |
| (ODO3866) | | 8120613 | |
| CC Margin (ODO3866) | 8.7 | Kidney Margin | 0.0 |
| | | 8120614 | |
| CC Gr.2 rectosigmoid | 13.5 | Kidney Cancer | 0.0 |
| (ODO3868) | | 9010320 | |
| CC Margin (ODO3868) | 9.5 | Kidney Margin | 0.0 |
| | | 9010321 | |
| CC Mod Diff (ODO3920) | 35.4 | Normal Uterus | 0.0 |
| CC Margin (ODO3920) | 57.4 | Uterus Cancer 064011 | 1.6 |
| CC Gr.2 ascend colon | 30.8 | Normal Thyroid | 11.8 |
| (ODO3921) | | | |
| CC Margin (ODO3921) | 6.4 | Thyroid Cancer | 0.3 |
| | | 064010 | |
| CC from Partial | 0.0 | Thyroid Cancer | 59.0 |
| Hepatectomy (ODO4309) | | A302152 | |
| Mets | | | |
| Liver Margin (ODO4309) | 5.3 | Thyroid Margin | 0.0 |
| | | A302153 | |
| Colon mets to lung | 0.0 | Normal Breast | 0.0 |
| (OD04451-01) | | | |
| Lung Margin (OD04451- | 0.4 | Breast Cancer | 0.0 |
| 02) | | (OD04566) | |
| Normal Prostate 6546-1 | 1.6 | Breast Cancer | 0.0 |
| | | (OD04590-01) | |
| Prostate Cancer | 21.8 | Breast Cancer Mets | 0.4 |
| (OD04410) | | (OD04590-03) | |
| Prostate Margin | 19.6 | Breast Cancer | 1.3 |
| (OD04410) | | Metastasis | |
| | | (OD04655-05) | |
| Prostate Cancer | 16.2 | Breast Cancer 064006 | 2.4 |
| (OD04720-01) | | | |
| Prostate Margin | 18.9 | Breast Cancer 1024 | 0.0 |
| (OD04720-02) | | | |
| Normal Lung 061010 | 73.7 | Breast Cancer | 10.7 |
| | | 9100266 | |
| Lung Met to Muscle | 4.0 | Breast Margin | 2.0 |
| (ODO4286) | | 9100265 | |
| Muscle Margin | 0.0 | Breast Cancer | 2.4 |
| (ODO4286) | | A209073 | |
| Lung Malignant Cancer | 0.0 | Breast Margin | 1.9 |
| (OD03126) | | A2090734 | |
| Lung Margin (OD03126) | 2.1 | Normal Liver | 2.6 |
| Lung Cancer (OD04404) | 0.4 | Liver Cancer 064003 | 4.5 |
| Lung Margin (OD04404) | 0.0 | Liver Cancer 1025 | 0.0 |
| Lung Cancer (OD04565) | 0.0 | Liver Cancer 1026 | 0.0 |
| Lung Margin (OD04565) | 2.5 | Liver Cancer 6004-T | 0.0 |
| Lung Cancer (OD04237- | 5.2 | Liver Tissue 6004-N | 1.5 |
| 01) | | | |
| Lung Margin (OD04237- | 1.9 | Liver Cancer 6005-T | 0.0 |
| 02) | | | |
| Ocular Mel Met to Liver | 0.4 | Liver Tissue 6005-N | 0.0 |
| (ODO4310) | | | |
| Liver Margin (ODO4310) | 2.7 | Normal Bladder | 18.6 |
| Melanoma Mets to Lung | 0.7 | Bladder Cancer 1023 | 0.0 |
| (OD04321) | | | |
| Lung Margin (OD04321) | 0.0 | Bladder Cancer | 12.6 |
| | | A302173 | |
| Normal Kidney | 0.0 | Bladder Cancer | 6.3 |
| | | (OD04718-01) | |
| Kidney Ca, Nuclear grade | 0.0 | Bladder Normal | 7.3 |
| 2 (OD04338) | | Adjacent (OD04718- | |
| | | 03) | |
| Kidney Margin | 0.0 | Normal Ovary | 0.0 |
| (OD04338) | | | |
| Kidney Ca Nuclear grade | 0.0 | Ovarian Cancer | 0.0 |
| 1/2 (OD04339) | | 064008 | |
| Kidney Margin | 0.5 | Ovarian Cancer | 0.0 |
| (OD04339) | | (OD04768-07) | |
| Kidney Ca, Clear cell | 0.0 | Ovary Margin | 0.0 |
| type (OD04340) | | (OD04768-08) | |
| Kidney Margin | 0.0 | Normal Stomach | 0.0 |
| (OD04340) | | | |
| Kidney Ca, Nuclear grade | 0.0 | Gastric Cancer | 2.0 |
| 3 (OD04348) | | 9060358 | |
| Kidney Margin | 1.0 | Stomach Margin | 13.1 |
| (OD04348) | | 9060359 | |
| Kidney Cancer | 0.0 | Gastric Cancer | 11.2 |
| (OD04622-01) | | 9060395 | |
| Kidney Margin | 0.0 | Stomach Margin | 15.9 |
| (OD04622-03) | | 9060394 | |
| Kidney Cancer | 0.5 | Gastric Cancer | 41.2 |
| (OD04450-01) | | 9060397 | |
| Kidney Margin | 0.0 | Stomach Margin | 5.6 |
| (OD04450-03) | | 9060396 | |
| Kidney Cancer 8120607 | 0.0 | Gastric Cancer | 100.0 |
| | | 064005 | |

TABLE BID
Panel 4D

| | Rel. Exp. (%) | | Rel. Exp. (%) |
|---|---|---|---|
| | Ag2913, Run | | Ag2913, Run |
| Tissue Name | 157366468 | Tissue Name | 157366468 |
| Secondary Th1 act | 14.9 | HUVEC IL-1beta | 0.0 |
| Secondary Th2 act | 27.7 | HUVEC IFN gamma | 0.0 |
| Secondary Tr1 act | 47.0 | HUVEC TNF alpha + IFN | 0.0 |
| | | gamma | |
| Secondary Th1 rest | 18.2 | HUVEC TNF alpha + IL4 | 0.9 |
| Secondary Th2 rest | 19.6 | HUVEC IL-11 | 1.3 |
| Secondary Tr1 rest | 13.9 | Lung Microvascular EC | 9.2 |
| | | none | |
| Primary Th1 act | 61.6 | Lung Microvascular EC | 9.1 |
| | | TNF alpha + IL-1beta | |
| Primary Th2 act | 56.3 | Microvascular Dermal EC | 10.4 |
| | | none | |
| Primary Tr1 act | 41.5 | Microsvasular Dermal EC | 7.8 |
| | | TNF alpha + IL-1beta | |
| Primary Th1 rest | 100.0 | Bronchial epithelium | 0.2 |
| | | TNF alpha + IL1beta | |
| Primary Th2 rest | 46.0 | Small airway epithelium | 0.5 |
| | | none | |
| Primary Tr1 rest | 16.8 | Small airway epithelium | 2.6 |
| | | TNF alpha + IL-1beta | |
| CD45RA CD4 | 9.9 | Coronery artery SMC rest | 0.1 |
| lymphocyte act | | | |
| CD45RO CD4 | 16.7 | Coronery artery SMC | 0.0 |
| lymphocyte act | | TNF alpha + IL-1beta | |
| CD8 lymphocyte act | 15.5 | Astrocytes rest | 0.6 |
| Secondary CD8 | 17.9 | Astrocytes TNF alpha + IL- | 0.5 |
| lymphocyte rest | | 1beta | |
| Secondary CD8 | 16.6 | KU-812 (Basophil) rest | 4.6 |
| lymphocyte act | | | |
| CD4 lymphocyte none | 16.2 | KU-812 (Basophil) | 18.0 |
| | | PMA/ionomycin | |
| 2ry Th1/Th2/Tr1_anti- | 29.9 | CCD1106 (Keratinocytes) | 7.2 |
| CD95 CH11 | | none | |
| LAK cells rest | 28.3 | CCD1106 (Keratinocytes) | 0.2 |
| | | TNF alpha + IL-1beta | |
| LAK cells IL-2 | 18.9 | Liver cirrhosis | 8.1 |
| LAK cells IL-2 + IL-12 | 6.0 | Lupus kidney | 3.4 |
| LAK cells IL-2 + IFN | 6.8 | NCI-H292 none | 4.3 |
| gamma | | | |
| LAK cells IL-2 + IL-18 | 3.5 | NCI-H292 IL-4 | 2.1 |
| LAK cells | 8.8 | NCI-H292 IL-9 | 1.8 |
| PMA/ionomycin | | | |
| NK Cells IL-2 rest | 4.6 | NCI-H292 IL-13 | 0.2 |
| Two Way MLR 3 day | 12.4 | NCI-H292 IFN gamma | 0.5 |
| Two Way MLR 5 day | 6.9 | HPAEC none | 2.6 |
| Two Way MLR 7 day | 5.1 | HPAEC TNF alpha + IL-1 | 6.3 |
| | | beta | |
| PBMC rest | 4.9 | Lung fibroblast none | 9.9 |
| PBMC PWM | 37.4 | Lung fibroblast TNF | 2.5 |
| | | alpha + IL-1beta | |
| PBMC PHA-L | 12.9 | Lung fibroblast IL-4 | 12.7 |
| Ramos (B cell) none | 11.3 | Lung fibroblast IL-9 | 8.7 |
| Ramos (B cell) | 13.7 | Lung fibroblast IL-13 | 12.1 |
| ionomycin | | | |
| B lymphocytes PWM | 1.1 | Lung fibroblast IFN | 9.3 |
| | | gamma | |
| B lymphocytes CD40L | 0.5 | Dermal fibroblast | 9.9 |
| and IL-4 | | CCD1070 rest | |
| EOL-1 dbcAMP | 0.4 | Dermal fibroblast | 29.3 |
| | | CCD1070 TNF alpha | |
| EOL-1 dbcAMP | 0.9 | Dermal fibroblast | 0.7 |
| PMA/ionomycin | | CCD1070 IL-1beta | |
| Dendritic cells none | 0.7 | Dermal fibroblast IFN | 1.4 |
| | | gamma | |
| Dendritic cells LPS | 1.5 | Dermal fibroblast IL-4 | 5.1 |
| Dendritic cells anti- | 4.7 | IBD Colitis 2 | 16.6 |
| CD40 | | | |
| Monocytes rest | 5.6 | IBD Crohn's | 7.9 |
| Monocytes LPS | 0.3 | Colon | 24.7 |
| Macrophages rest | 3.6 | Lung | 13.4 |
| Macrophages LPS | 0.4 | Thymus | 72.7 |
| HUVEC none | 1.2 | Kidney | 65.1 |
| HUVEC starved | 1.2 | | |

CNS_neurodegeneration_v1.0 Summary: Ag2913 No significant expression detected. Potential failed chemistry reaction or bad probe/primer set (data not shown).

Panel 1.3D Summary: Ag2913 The NOV66 gene represents a novel G-protein coupled receptor (GPCR) with expression in the brain. The GPCR family of receptors contains a large number of neurotransmitter receptors, including the dopamine, serotonin, a and b-adrenergic, acetylcholine muscarinic, histamine, peptide, and metabotropic glutamate receptors. GPCRs are excellent drug targets in various neurologic and psychiatric diseases. All antipsychotics have been shown to act at the dopamine D2 receptor; similarly novel antipsychotics also act at the serotonergic receptor, and often the muscarinic and adrenergic receptors as well. While the majority of antidepressants can be classified as selective serotonin reuptake inhibitors, blockade of the 5-HT1A and a2 adrenergic receptors increases the effects of these drugs. The GPCRs are also of use as drug targets in the treatment of stroke. Blockade of the glutamate receptors may decrease the neuronal death resulting from excitotoxicity; further more the purinergic receptors have also been implicated as drug targets in the treatment of cerebral ischemia. The b-adrenergic receptors have been implicated in the treatment of ADHD with Ritalin, while the a-adrenergic receptors have been implicated in memory. Therefore this gene may be of use as a small molecule target for the treatment of any of the described diseases.

In addition, this gene is expressed in clusters of cell lines derived from lung cancer and colon cancer. Thus, expression of this gene could be used to differentiate between these sample and other samples on this panel and as a marker to detect the presence of colon and lung cancers. Furthermore, therapeutic modulation of the expression or function of this gene may be effective in the treatment of lung and colon cancers.

References:

El Yacoubi M, Ledent C, Parmentier M, Bertorelli R, Ongini E, Costentin J, Vaugeois J M. Adenosine A2A receptor antagonists are potential antidepressants: evidence based on pharmacology and A2A receptor knockout mice. Br J Pharmacol September 2001;134(1):68-77

1.Adenosine, an ubiquitous neuromodulator, and its analogues have been shown to produce ‘depressant’ effects in animal models believed to be relevant to depressive disorders, while adenosine receptor antagonists have been found to reverse adenosine-mediated ‘depressant’ effect. 2. We have designed studies to assess whether adenosine A2A receptor antagonists, or genetic inactivation of the receptor would be effective in established screening procedures, such as tail suspension and forced swim tests, which are predictive of clinical antidepressant activity. 3. Adenosine A2A receptor knockout mice were found to be less sensitive to ‘depressant’ challenges than their wildtype littermates. Consistently, the adenosine A2A receptor blockers SCH 58261 (1-10 mg kg(−1), i.p.) and KW 6002 (0.1-10 mg kg(−1), p.o.) reduced the total immobility time in the tail suspension test. 4. The efficacy of adenosine A2A receptor antagonists in reducing immobility time in the tail suspension test was confirmed and extended in two groups of mice. Specifically, SCH 58261 (1-10 mg kg(−1)) and ZM 241385 (15-60 mg kg(−1)) were effective in mice previously screened for having high immobility time, while SCH 58261 at 10 mg kg(−1) reduced immobility of mice that were selectively bred for their spontaneous ‘helplessness’ in this assay. 5. Additional experiments were carried out using the forced swim test. SCH 58261 at 10 mg kg(−1) reduced the immobility time by 61%, while KW 6002 decreased the total immobility time at the doses of 1 and 10 mg kg(−1) by 75 and 79%, respectively. 6.Administration of the dopamine D2 receptor antagonist haloperidol (50-200 microg kg(−1) i.p.) prevented the antidepressant-like effects elicited by SCH 58261 (10 mg kg(−1) i.p.) in forced swim test whereas it left unaltered its stimulant motor effects. 7. In conclusion, these data support the hypothesis that A2A receptor antagonists prolong escape-directed behaviour in two screening tests for antidepressants. Altogether the results support the hypothesis that blockade of the adenosine A2A receptor might be an interesting target for the development of effective antidepressant agents.

Blier P. Pharmacology of rapid-onset antidepressant treatment strategies. Clin Psychiatry 2001;62 Suppl 15: 12-7

Although selective serotonin reuptake inhibitors (SSRIs) block serotonin (5-HT) reuptake rapidly, their therapeutic action is delayed. The increase in synaptic 5-HT activates feedback mechanisms mediated by 5-HT1A (cell body) and 5-HT1 B (terminal) autoreceptors, which, respectively, reduce the firing in 5-HT neurons and decrease the amount of 5-HT released per action potential resulting in attenuated 5-HT neurotransmission. Long-term treatment desensitizes the inhibitory 5-HT1 autoreceptors, and 5-HT neurotransmission is enhanced. The time course of these events is similar to the delay of clinical action. The addition of pindolol, which blocks 5-HT1A receptors, to SSRI treatment decouples the feedback inhibition of 5-HT neuron firing and accelerates and enhances the antidepressant response. The neuronal circuitry of the 5-HT and norepinephrine (NE) systems and their connections to forebrain areas believed to be involved in depression has been dissected. The firing of 5-HT neurons in the raphe nuclei is driven, at least partly, by alpha1-adrenoceptor-mediated excitatory inputs from NE neurons. Inhibitory alpha2-adrenoceptors on the NE neuroterminals form part of a feedback control mechanism. Mirtazapine, an antagonist at alpha2-adrenoceptors, does not enhance 5-HT neurotransmission directly but disinhibits the NE activation of 5-HT neurons and thereby increases 5-HT neurotransmission by a mechanism that does not require a time-dependent desensitization of receptors. These neurobiological phenomena may underlie the apparently faster onset of action of mirtazapine compared with the SSRIs.

Tranquillini M E, Reggiani A. Glycine-site antagonists and stroke. Expert Opin Investig Drugs November 1999;8(11):1837-1848

The excitatory amino acid, (S)-glutamic acid, plays an important role in controlling many neuronal processes. Its action is mediated by two main groups of receptors:,the ionotropic receptors (which include NMDA, AMPA and kainic acid subtypes) and the metabotropic receptors (mGluR(1-8)) mediating G-protein coupled responses. This review focuses on the strychnine insensitive glycine binding site located on the NMDA receptor channel, and on the possible use of selective antagonists for the treatment of stroke. Stroke is a devastating disease caused by a sudden vascular accident. Neurochemically, a massive release of glutamate occurs in neuronal tissue; this overactivates the NMDA receptor, leading to increased intracellular calcium influx, which causes neuronal cell death through necrosis. NMDA receptor activation strongly depends upon the presence of glycine as a co-agonist. Therefore, the administration of a glycine antagonist can block overactivation of NMDA receptors, thus preserving neurones from damage. The glycine antagonists currently identified can be divided into five main categories depending on their chemical structure: indoles, tetrahydroquinolines, benzoazepines, quinoxalinediones and pyrida-zinoquinolines.

Monopoli A, Lozza G, Forlani A, Mattavelli A, Ongini E. Blockade of adenosine A2A receptors by SCH 58261 results in neuroprotective effects in cerebral ischaemia in rats. Neuroreport Dec. 1, 1998;9(17):3955-9

Blockade of adenosine receptors can reduce cerebral infarct size in the model of global ischaemia. Using the potent and selective A2A adenosine receptor antagonist, SCH 58261, we assessed whether A2A receptors are involved in the neuronal damage following focal cerebral ischaemia as induced by occluding the left middle cerebral artery. SCH 58261 (0.01 mg/kg either i.p. or i.v.) administered to normotensive rats 10 min after ischaemia markedly reduced cortical infarct volume as measured 24 h later (30% vs controls, p<0.05). Similar effects were observed when SCH 58261 (0.01 mg/kg, i.p.) was administered to hypertensive rats (28% infarct volume reduction vs controls, p<0.05). Neuroprotective properties of SCH 58261 administered after ischaemia indicate that blockade of A2A adenosine receptors is a potentially useful biological target for the reduction of brain injury.

Panel 2D Summary: Ag2913 The NOV66 gene is a diagnostic marker for gastric thyroid and bladder cancer and a target for therapeutic intervention in gastric, thyroid and bladder cancer through the use of antibodies or small molecule drugs. This is based on the expression profile of this gene that shows higher expression in some gastric, thyroid and bladder cancer samples compared to normal tissues.

Panel 4D Summary: Ag2913 The NOV66 gene, an olfactory receptor homolog is expressed at moderate levels in activated and resting T lymphocytes (CT range 30.13-32.98). Small molecules or therapeutic antibodies that antagonize the function of the NOV66 gene prodcut may reduce or eliminate the symptoms of autoimmune and inflammatory diseases, including Crohn's disease, ulcerative colitis, multiple sclerosis, chronic obstructive pulmonary disease, asthma, emphysema, rheumatoid arthritis, lupus erythematosus, or psoriasis.

Panel CNS_{—}1 Summary: Ag2913 No significant expression detected. Potential probe/primer failure (data not shown).

NOV67

Expression of gene NOV67 was assessed using the primer-probe set Ag2951, described in Table BJA. Results of the RTO-PCT runs are shown in Tables BJB and BJC.

TABLE BJA
Probe Name Ag2951

| | | | Start | |
|---|---|---|---|---|
| Primers | Sequences | Length | Position | SEQ ID NO: |
| Forward | 5′-acctctcacttgtggacatctg-3′ | 22 | 243 | 1270 |
| Probe | TET-5′-tacacctccagcagggtccctcagat-3′-TAMRA | 26 | 266 | 1271 |
| Reverse | 5′-ggcaaaggagatggtctttct-3′ | 21 | 314 | 1272 |

TABLE BJB
AI_comprehensive panel_v1.0

| | Rel. Exp. (%) | | Rel. Exp. (%) |
|---|---|---|---|
| | Ag2951, Run | | Ag2951, Run |
| Tissue Name | 248065290 | Tissue Name | 248065290 |
| 110967 COPD-F | 3.1 | 112427 Match Control | 49.3 |
| | | Psoriasis-F | |
| 110980 COPD-F | 4.9 | 112418 Psoriasis-M | 13.6 |
| 110968 COPD-M | 2.4 | 112723 Match Control | 0.0 |
| | | Psoriasis-M | |
| 110977 COPD-M | 45.7 | 112419 Psoriasis-M | 20.2 |
| 110989 Emphysema-F | 25.9 | 112424 Match Control | 9.5 |
| | | Psoriasis-M | |
| 110992 Emphysema-F | 33.9 | 112420 Psoriasis-M | 93.3 |
| 110993 Emphysema-F | 5.8 | 112425 Match Control | 32.8 |
| | | Psoriasis-M | |
| 110994 Emphysema-F | 0.0 | 104689 (MF) OA | 0.0 |
| | | Bone-Backus | |
| 110995 Emphysema-F | 55.5 | 104690 (MF) Adj | 0.0 |
| | | “Normal” Bone-Backus | |
| 110996 Emphysema-F | 7.0 | 104691 (MF) OA | 0.0 |
| | | Synovium-Backus | |
| 110997 Asthma-M | 12.8 | 104692 (BA) OA | 0.0 |
| | | Cartilage-Backus | |
| 111001 Asthma-F | 18.6 | 104694 (BA) OA | 0.0 |
| | | Bone-Backus | |
| 111002 Asthma-F | 27.7 | 104695 (BA) Adj | 5.5 |
| | | “Normal” Bone-Backus | |
| 111003 Atopic | 16.7 | 104696 (BA) OA | 10.2 |
| Asthma-F | | Synovium-Backus | |
| 111004 Atopic | 37.4 | 104700 (SS) OA Bone- | 7.1 |
| Asthma-F | | Backus | |
| 111005 Atopic | 31.6 | 104701 (SS) Adj | 14.3 |
| Asthma-F | | “Normal” Bone-Backus | |
| 111006 Atopic | 1.8 | 104702 (SS) OA | 28.3 |
| Asthma-F | | Synovium-Backus | |
| 111417 Allergy-M | 20.4 | 117093 OA Cartilage | 65.5 |
| | | Rep7 | |
| 112347 Allergy-M | 2.5 | 112672 OA Bone5 | 26.6 |
| 112349 Normal Lung-F | 0.0 | 112673 OA Synovium5 | 21.6 |
| 112357 Normal Lung-F | 12.8 | 112674 OA Synovial | 9.3 |
| | | Fluid cells5 | |
| 112354 Normal Lung-M | 4.5 | 117100 OA Cartilage | 2.9 |
| | | Rep14 | |
| 112374 Crohns-F | 16.3 | 112756 OA Bone9 | 7.3 |
| 112389 Match | 15.0 | 112757 OA Synovium9 | 3.3 |
| Control Crohns-F | | | |
| 112375 Crohns-F | 7.5 | 112758 OA Synovial | 12.9 |
| | | Fluid Cells9 | |
| 112732 Match | 41.5 | 117125 RA Cartilage | 0.0 |
| Control Crohns-F | | Rep2 | |
| 112725 Crohns-M | 2.0 | 113492 Bone2 RA | 8.5 |
| 112387 Match | 5.3 | 113493 Synovium2 RA | 1.9 |
| Control Crohns-M | | | |
| 112378 Crohns-M | 4.0 | 113494 Syn Fluid Cells | 7.6 |
| | | RA | |
| 112390 Match | 61.6 | 113499 Cartilage4 RA | 13.7 |
| Control Crohns-M | | | |
| 112726 Crohns-M | 21.3 | 113500 Bone4 RA | 8.3 |
| 112731 Match | 14.6 | 113501 Synovium4 RA | 20.4 |
| Control Crohns-M | | | |
| 112380 Ulcer Col-F | 18.3 | 113502 Syn Fluid | 6.5 |
| | | Cells4 RA | |
| 112734 Match | 100.0 | 113495 Cartilage3 RA | 11.7 |
| Control Ulcer Col-F | | | |
| 112384 Ulcer Col-F | 60.7 | 113496 Bone3 RA | 4.4 |
| 112737 Match | 22.1 | 113497 Synovium3 RA | 3.0 |
| Control Ulcer Col-F | | | |
| 112386 Ulcer Col-F | 4.6 | 113498 Syn Fluid | 17.3 |
| | | Cells3 RA | |
| 112738 Match | 3.7 | 117106 Normal | 11.4 |
| Control Ulcer Col-F | | Cartilage Rep20 | |
| 112381 Ulcer Col-M | 0.0 | 113663 Bone3 Normal | 0.0 |
| 112735 Match | 15.4 | 113664 Synovium3 | 0.0 |
| Control Ulcer Col-M | | Normal | |
| 112382 Ulcer Col-M | 58.2 | 113665 Syn Fluid | 1.3 |
| | | Cells3 Normal | |
| 112394 Match | 4.2 | 117107 Normal | 0.0 |
| Control Ulcer Col-M | | Cartilage Rep22 | |
| 112383 Ulcer Col-M | 63.3 | 113667 Bone4 Normal | 39.5 |
| 112736 Match | 12.1 | 113668 Synovium4 | 25.5 |
| Control Ulcer Col-M | | Normal | |
| 112423 Psoriasis-F | 26.2 | 113669 Syn Fluid | 26.1 |
| | | Cells4 Normal | |

TABLE BJC
Panel 4D

| | Rel. Exp. (%) | | Rel. Exp. (%) |
|---|---|---|---|
| | Ag2951, Run | | Ag2951, Run |
| Tissue Name | 164403342 | Tissue Name | 164403342 |
| Secondary Th1 act | 0.0 | HUVEC IL-1beta | 0.0 |
| Secondary Th2 act | 0.0 | HUVEC IFN gamma | 0.8 |
| Secondary Tr1 act | 0.0 | HUVEC TNF alpha + IFN | 0.0 |
| | | gamma | |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC | 0.0 |
| | | none | |
| Primary Th1 act | 0.7 | Lung Microvascular EC | 0.0 |
| | | TNF alpha + IL-1beta | |
| Primary Th2 act | 0.0 | Microvascular Dermal EC | 0.0 |
| | | none | |
| Primary Tr1 act | 0.0 | Microsvasular Dermal EC | 0.0 |
| | | TNF alpha + IL-1beta | |
| Primary Th1 rest | 1.3 | Bronchial epithelium | 1.0 |
| | | TNF alpha + IL1beta | |
| Primary Th2 rest | 7.0 | Small airway epithelium | 0.0 |
| | | none | |
| Primary Tr1 rest | 17.2 | Small airway epithelium | 5.2 |
| | | TNF alpha + IL-1beta | |
| CD45RA CD4 | 0.0 | Coronery artery SMC rest | 0.0 |
| lymphocyte act | | | |
| CD45RO CD4 | 1.5 | Coronery artery SMC | 0.0 |
| lymphocyte act | | TNF alpha + IL-1beta | |
| CD8 lymphocyte act | 0.0 | Astrocytes rest | 0.0 |
| Secondary CD8 | 0.0 | Astrocytes TNF alpha + | 0.0 |
| lymphocyte rest | | IL-1beta | |
| Secondary CD8 | 0.0 | KU-812 (Basophil) rest | 1.0 |
| lymphocyte act | | | |
| CD4 lymphocyte none | 13.3 | KU-812 (Basophil) | 0.0 |
| | | PMA/ionomycin | |
| 2ry Th1/Th2/Tr1_anti- | 0.9 | CCD1106 (Keratinocytes) | 1.2 |
| CD95 CH11 | | none | |
| LAK cells rest | 11.8 | CCD1106 (Keratinocytes) | 0.0 |
| | | TNF alpha + IL-1beta | |
| LAK cells IL-2 | 0.0 | Liver cirrhosis | 7.7 |
| LAK cells IL-2 + IL-12 | 0.0 | Lupus kidney | 0.0 |
| LAK cells IL-2 + IFN | 3.2 | NCI-H292 none | 2.4 |
| gamma | | | |
| LAK cells IL-2 + IL-18 | 4.6 | NCI-H292 IL-4 | 1.9 |
| LAK cells | 0.0 | NCI-H292 IL-9 | 0.0 |
| PMA/ionomycin | | | |
| NK Cells IL-2 rest | 0.0 | NCI-H292 IL-13 | 0.0 |
| Two Way MLR 3 day | 0.0 | NCI-H292 IFN gamma | 0.0 |
| Two Way MLR 5 day | 1.6 | HPAEC none | 0.0 |
| Two Way MLR 7 day | 0.0 | HPAEC TNF alpha + IL-1 | 0.0 |
| | | beta | |
| PBMC rest | 7.6 | Lung fibroblast none | 0.0 |
| PBMC PWM | 100.0 | Lung fibroblast TNF | 0.0 |
| | | alpha + IL-1beta | |
| PBMC PHA-L | 5.8 | Lung fibroblast IL-4 | 0.0 |
| Ramos (B cell) none | 0.0 | Lung fibroblast IL-9 | 1.7 |
| Ramos (B cell) | 0.0 | Lung fibroblast IL-13 | 0.0 |
| ionomycin | | | |
| B lymphocytes PWM | 1.0 | Lung fibroblast IFN | 0.0 |
| | | gamma | |
| B lymphocytes CD40L | 5.2 | Dermal fibroblast | 0.0 |
| and IL-4 | | CCD1070 rest | |
| EOL-1 dbcAMP | 19.9 | Dermal fibroblast | 1.4 |
| | | CCD1070 TNF alpha | |
| EOL-1 dbcAMP | 7.8 | Dermal fibroblast | 0.0 |
| PMA/ionomycin | | CCD1070 IL-1beta | |
| Dendritic cells none | 9.2 | Dermal fibroblast IFN | 0.0 |
| | | gamma | |
| Dendritic cells LPS | 2.9 | Dermal fibroblast IL-4 | 0.0 |
| Dendritic cells anti- | 3.0 | IBD Colitis 2 | 1.8 |
| CD40 | | | |
| Monocytes rest | 26.8 | IBD Crohn's | 0.0 |
| Monocytes LPS | 0.0 | Colon | 2.5 |
| Macrophages rest | 2.4 | Lung | 1.2 |
| Macrophages LPS | 0.0 | Thymus | 0.8 |
| HUVEC none | 0.0 | Kidney | 8.7 |
| HUVEC starved | 0.0 | | |

AI_comprehensive panel_v_{1.0} Summary: Ag2951 Highest expression of the NOV67 gene is seen in normal tissue adjacent to colon from an ulcerative colitis patient(CT=33). Thus, expression of this gene could be used to distinguish this sample from other samples on this panel. Please see Panel 4D for further discussion of utility of this gene inflammation.

CNS_neurodegeneration_v1.0 Summary: Ag2951 Expression of this gene is low/undetectable (CTs>35) across all of the samples on this panel (data not shown).

Panel 1.3D Summary: Ag2951 Expression of this gene is low/undetectable(CTs>35) across all of the samples on this panel (data not shown).

Panel 4.1D Summary: Ag2951 Expression of this gene is low/undetectable (CTs>35) across all of the samples on this panel (data not shown).

Panel 4D Summary: Ag2951 The NOV67 gene is expressed at a moderate level (CT=32.78) in pokeweed mitogen-stimulated peripheral blood leukocytes, consisting primarily of activated B lymphocytes. Small molecule antagonists or theurapeutic antibody antagonists that block the function of the CG56571-gene product may be useful in several autoimmune and inflammatory diseases in which activated B cells can play major roles as sources of autoantibody-producing cells and as powerful antigen-presenting cells including, but not limited to, Crohn's disease, ulcerative colitis, multiple sclerosis, chronic obstructive pulmonary disease, asthma, emphysema, rheumatoid arthritis, lupus erythematosus, or psoriasis.

NOV69a and NOV69b

Expression of gene NOV69a and variant NOV69b was assessed using the primer-probe sets Ag2460 and Ag349, described in Tables BKA and BKB. Results of the RTQ-PCR runs are shown in Tables BKC, BKD, BKE and BKF.

TABLE BKA
Probe Name Ag2460

| | | | Start | |
|---|---|---|---|---|
| Primers | Sequences | Length | Position | SEQ ID NO: |
| Forward | 5′-tcatagcagtcccgaggaa-3′ | 19 | 89 | 1273 |
| Probe | TET-5′-tcactattgccttaatctcatgccga-3′-TAMRA | 26 | 125 | 1274 |
| Reverse | 5′-ttctcaagggtctccacatg-3′ | 20 | 151 | 1275 |

TABLE BKB
Probe Name Ag349

| | | | Start | |
|---|---|---|---|---|
| Primers | Sequences | Length | Position | SEQ ID NO: |
| Forward | 5′-gggaaagccacagactcgaa-3′ | 20 | 289 | 1276 |
| Probe | TET-5′-cttctaccacagccagagtggcaggaact-3′-TAMRA | 29 | 255 | 1277 |
| Reverse | 5′-acccgagcctgtgaagtcct-3′ | 20 | 231 | 1278 |

TABLE BKC
Panel 1

| | Rel. Exp. (%) | | Rel. Exp. (%) |
|---|---|---|---|
| | Ag349, Run | | Ag349, Run |
| Tissue Name | 97804233 | Tissue Name | 97804233 |
| Endothelial cells | 0.0 | Renal ca. 786-0 | 0.0 |
| Endothelial cells | 0.0 | Renal ca. A498 | 0.2 |
| (treated) | | | |
| Pancreas | 0.0 | Renal ca. RXF 393 | 0.0 |
| Pancreatic ca. CAPAN 2 | 0.0 | Renal ca. ACHN | 0.0 |
| Adrenal gland | 0.0 | Renal ca. UO-31 | 0.0 |
| Thyroid | 0.5 | Renal ca. TK-10 | 0.0 |
| Salivary gland | 25.5 | Liver | 0.0 |
| Pituitary gland | 0.0 | Liver (fetal) | 0.0 |
| Brain (fetal) | 0.0 | Liver ca. (hepatoblast) | 0.0 |
| | | HepG2 | |
| Brain (whole) | 0.0 | Lung | 0.0 |
| Brain (amygdala) | 0.0 | Lung (fetal) | 0.0 |
| Brain (cerebellum) | 0.0 | Lung ca. (small cell) | 0.0 |
| | | LX-1 | |
| Brain (hippocampus) | 0.0 | Lung ca. (small cell) | 1.5 |
| | | NCI-H69 | |
| Brain (substantia nigra) | 0.0 | Lung ca. (s.cell var.) | 0.0 |
| | | SHP-77 | |
| Brain (thalamus) | 0.0 | Lung ca. (large | 1.3 |
| | | cell)NCI-H460 | |
| Brain (hypothalamus) | 0.0 | Lung ca. (non-sm. | 0.0 |
| | | cell) A549 | |
| Spinal cord | 0.0 | Lung ca. (non-s.cell) | 0.0 |
| | | NCI-H23 | |
| glio/astro U87-MG | 59.0 | Lung ca. (non-s.cell) | 0.0 |
| | | HOP-62 | |
| glio/astro U-118-MG | 0.0 | Lung ca. (non-s.cl) | 0.0 |
| | | NCI-H522 | |
| astrocytoma SW1783 | 0.0 | Lung ca. (squam.) SW | 0.0 |
| | | 900 | |
| neuro*; met SK-N-AS | 0.0 | Lung ca. (squam.) | 0.0 |
| | | NCI-H596 | |
| astrocytoma SF-539 | 0.0 | Mammary gland | 0.0 |
| astrocytoma SNB-75 | 0.0 | Breast ca.* (pl.ef) | 0.0 |
| | | MCF-7 | |
| glioma SNB-19 | 3.1 | Breast ca.* (pl.ef) | 0.0 |
| | | MDA-MB-231 | |
| glioma U251 | 0.0 | Breast ca.* (pl. ef) | 0.0 |
| | | T47D | |
| glioma SF-295 | 0.0 | Breast ca. BT-549 | 0.0 |
| Heart | 0.0 | Breast ca. MDA-N | 0.0 |
| Skeletal muscle | 0.0 | Ovary | 0.0 |
| Bone marrow | 0.0 | Ovarian ca. OVCAR-3 | 0.0 |
| Thymus | 66.4 | Ovarian ca. OVCAR-4 | 0.0 |
| Spleen | 0.1 | Ovarian ca. OVCAR-5 | 1.5 |
| Lymph node | 3.1 | Ovarian ca. OVCAR-8 | 0.0 |
| Colon (ascending) | 29.9 | Ovarian ca. IGROV-1 | 0.0 |
| Stomach | 77.4 | Ovarian ca. (ascites) | 0.0 |
| | | SK-OV-3 | |
| Small intestine | 0.0 | Uterus | 0.0 |
| Colon ca. SW480 | 0.0 | Placenta | 0.0 |
| Colon ca.* SW620 | 0.0 | Prostate | 0.0 |
| (SW480 met) | | | |
| Colon ca. HT29 | 0.0 | Prostate ca.* (bone | 0.0 |
| | | met) PC-3 | |
| Colon ca. HCT-116 | 0.0 | Testis | 0.0 |
| Colon ca. CaCo-2 | 0.0 | Melanoma | 0.0 |
| | | Hs688(A).T | |
| Colon ca. HCT-15 | 0.0 | Melanoma* (met) | 0.0 |
| | | Hs688(B).T | |
| Colon ca. HCC-2998 | 0.0 | Melanoma UACC-62 | 0.0 |
| Gastric ca.* (liver met) | 0.0 | Melanoma M14 | 0.0 |
| NCI-N87 | | | |
| Bladder | 0.0 | Melanoma LOX | 0.0 |
| | | IMVI | |
| Trachea | 15.5 | Melanoma* (met) SK- | 0.0 |
| | | MEL-5 | |
| Kidney | 0.0 | Melanoma* SK-MEL- | 100.0 |
| | | 28 | |
| Kidney (fetal) | 0.0 | | |

TABLE BKD
Panel 1.3D

| | Rel. Exp. (%) | | Rel. Exp. (%) |
|---|---|---|---|
| | Ag2460, Run | | Ag2460, Run |
| Tissue Name | 157914666 | Tissue Name | 157914666 |
| Liver adenocarcinoma | 0.0 | Kidney (fetal) | 0.0 |
| Pancreas | 0.0 | Renal ca. 786-0 | 0.0 |
| Pancreatic ca. CAPAN 2 | 0.0 | Renal ca. A498 | 9.6 |
| Adrenal gland | 0.0 | Renal ca. RXF 393 | 0.0 |
| Thyroid | 5.6 | Renal ca. ACHN | 0.0 |
| Salivary gland | 19.8 | Renal ca. UO-31 | 0.0 |
| Pituitary gland | 0.0 | Renal ca. TK-10 | 0.0 |
| Brain (fetal) | 0.0 | Liver | 0.0 |
| Brain (whole) | 0.0 | Liver (fetal) | 0.0 |
| Brain (amygdala) | 0.0 | Liver ca. | 0.0 |
| | | (hepatoblast) HepG2 | |
| Brain (cerebellum) | 0.0 | Lung | 2.2 |
| Brain (hippocampus) | 0.0 | Lung (fetal) | 0.0 |
| Brain (substantia nigra) | 0.0 | Lung ca. (small cell) | 0.0 |
| | | LX-1 | |
| Brain (thalamus) | 0.0 | Lung ca. (small cell) | 0.0 |
| | | NCI-H69 | |
| Cerebral Cortex | 0.0 | Lung ca. (s.cell var.) | 0.0 |
| | | SHP-77 | |
| Spinal cord | 45.7 | Lung ca. (large | 0.0 |
| | | cell)NCI-H460 | |
| glio/astro U87-MG | 2.4 | Lung ca. (non-sm. | 0.0 |
| | | cell) A549 | |
| glio/astro U-118-MG | 2.5 | Lung ca. (non-s.cell) | 0.0 |
| | | NCI-H23 | |
| astrocytoma SW1783 | 0.0 | Lung ca. (non-s.cell) | 0.0 |
| | | HOP-62 | |
| neuro*; met SK-N-AS | 0.0 | Lung ca. (non-s.cl) | 0.0 |
| | | NCI-H522 | |
| astrocytoma SF-539 | 0.0 | Lung ca. (squam.) | 0.0 |
| | | SW 900 | |
| astrocytoma SNB-75 | 0.0 | Lung ca. (squam.) | 0.0 |
| | | NCI-H596 | |
| glioma SNB-19 | 0.0 | Mammary gland | 100.0 |
| glioma U251 | 0.0 | Breast ca.* (pl.ef) | 0.0 |
| | | MCF-7 | |
| glioma SF-295 | 0.0 | Breast ca.* (pl.ef) | 0.0 |
| | | MDA-MB-231 | |
| Heart (fetal) | 0.0 | Breast ca.* (pl.ef) | 0.0 |
| | | T47D | |
| Heart | 0.0 | Breast ca. BT-549 | 0.0 |
| Skeletal muscle (fetal) | 0.0 | Breast ca. MDA-N | 0.0 |
| Skeletal muscle | 0.0 | Ovary | 0.0 |
| Bone marrow | 0.0 | Ovarian ca. OVCAR-3 | 0.0 |
| Thymus | 61.1 | Ovarian ca. OVCAR-4 | 0.0 |
| Spleen | 0.0 | Ovarian ca. OVCAR-5 | 0.0 |
| Lymph node | 8.4 | Ovarian ca. OVCAR-8 | 0.0 |
| Colorectal | 0.0 | Ovarian ca. IGROV-1 | 0.0 |
| Stomach | 77.4 | Ovarian ca.* (ascites) | 0.0 |
| | | SK-OV-3 | |
| Small intestine | 0.0 | Uterus | 0.0 |
| Colon ca. SW480 | 0.0 | Placenta | 0.0 |
| Colon ca.* | 0.0 | Prostate | 2.3 |
| SW620(SW480 met) | | | |
| Colon ca. HT29 | 0.0 | Prostate ca.* (bone | 0.0 |
| | | met)PC-3 | |
| Colon ca. HCT-116 | 0.0 | Testis | 0.0 |
| Colon ca. CaCo-2 | 0.0 | Melanoma | 0.0 |
| | | Hs688(A).T | |
| Colon ca. | 0.0 | Melanoma* (met) | 0.0 |
| tissue(ODO3866) | | Hs688(B).T | |
| Colon ca. HCC-2998 | 0.0 | Melanoma UACC-62 | 0.0 |
| Gastric ca.* (liver met) | 0.0 | Melanoma M14 | 0.0 |
| NCI-N87 | | | |
| Bladder | 0.0 | Melanoma LOX | 0.0 |
| | | IMVI | |
| Trachea | 59.5 | Melanoma* (met) | 0.0 |
| | | SK-MEL-5 | |
| Kidney | 0.0 | Adipose | 0.0 |

TABLE BKE
Panel 2D

| | Rel. Exp. (%) | | Rel. Exp. (%) |
|---|---|---|---|
| | Ag2460, Run | | Ag2460, Run |
| Tissue Name | 157914720 | Tissue Name | 157914720 |
| Normal Colon | 0.0 | Kidney Margin | 0.0 |
| | | 8120608 | |
| CC Well to Mod Diff | 0.0 | Kidney Cancer | 0.0 |
| (ODO3866) | | 8120613 | |
| CC Margin (ODO3866) | 0.0 | Kidney Margin | 0.0 |
| | | 8120614 | |
| CC Gr.2 rectosigmoid | 0.0 | Kidney Cancer | 0.0 |
| (ODO3868) | | 9010320 | |
| CC Margin (ODO3868) | 0.0 | Kidney Margin | 0.0 |
| | | 9010321 | |
| CC Mod Diff (ODO3920) | 0.0 | Normal Uterus | 0.0 |
| CC Margin (ODO3920) | 0.0 | Uterus Cancer 064011 | 0.0 |
| CC Gr.2 ascend colon | 0.0 | Normal Thyroid | 0.9 |
| (ODO3921) | | | |
| CC Margin (ODO3921) | 0.0 | Thyroid Cancer | 0.0 |
| | | 064010 | |
| CC from Partial | 0.0 | Thyroid Cancer | 0.0 |
| Hepatectomy (ODO4309) | | A302152 | |
| Mets | | | |
| Liver Margin (ODO4309) | 0.0 | Thyroid Margin | 0.0 |
| | | A302153 | |
| Colon mets to lung | 0.0 | Normal Breast | 0.0 |
| (OD04451-01) | | | |
| Lung Margin (OD04451- | 0.0 | Breast Cancer | 0.0 |
| 02) | | (OD04566) | |
| Normal Prostate 6546-1 | 0.0 | Breast Cancer | 0.0 |
| | | (OD04590-01) | |
| Prostate Cancer | 0.0 | Breast Cancer Mets | 0.2 |
| (OD04410) | | (OD04590-03) | |
| Prostate Margin | 0.0 | Breast Cancer | 0.6 |
| (OD04410) | | Metastasis | |
| | | (OD04655-05) | |
| Prostate Cancer | 0.0 | Breast Cancer 064006 | 0.0 |
| (OD04720-01) | | | |
| Prostate Margin | 0.0 | Breast Cancer 1024 | 0.0 |
| (OD04720-02) | | | |
| Normal Lung 061010 | 0.4 | Breast Cancer | 0.0 |
| | | 9100266 | |
| Lung Met to Muscle | 0.0 | Breast Margin | 0.0 |
| (ODO4286) | | 9100265 | |
| Muscle Margin | 0.0 | Breast Cancer | 0.0 |
| (ODO4286) | | A209073 | |
| Lung Malignant Cancer | 0.0 | Breast Margin | 0.0 |
| (OD03126) | | A2090734 | |
| Lung Margin (OD03126) | 0.0 | Normal Liver | 0.0 |
| Lung Cancer (OD04404) | 100.0 | Liver Cancer 064003 | 0.0 |
| Lung Margin (OD04404) | 0.0 | Liver Cancer 1025 | 0.1 |
| Lung Cancer (OD04565) | 0.5 | Liver Cancer 1026 | 0.0 |
| Lung Margin (OD04565) | 0.0 | Liver Cancer 6004-T | 0.0 |
| Lung Cancer (OD04237- | 0.0 | Liver Tissue 6004-N | 0.0 |
| 01) | | | |
| Lung Margin (OD04237- | 0.0 | Liver Cancer 6005-T | 0.0 |
| 02) | | | |
| Ocular Mel Met to Liver | 0.0 | Liver Tissue 6005-N | 0.0 |
| (ODO4310) | | | |
| Liver Margin (ODO4310) | 0.0 | Normal Bladder | 0.0 |
| Melanoma Mets to Lung | 0.0 | Bladder Cancer 1023 | 0.0 |
| (OD04321) | | | |
| Lung Margin (OD04321) | 0.0 | Bladder Cancer | 0.0 |
| | | A302173 | |
| Normal Kidney | 0.0 | Bladder Cancer | 0.0 |
| | | (OD04718-01) | |
| Kidney Ca, Nuclear grade | 0.0 | Bladder Normal | 0.0 |
| 2 (OD04338) | | Adjacent (OD04718- | |
| | | 03) | |
| Kidney Margin | 0.0 | Normal Ovary | 0.0 |
| (OD04338) | | | |
| Kidney Ca Nuclear grade | 0.0 | Ovarian Cancer | 0.0 |
| 1/2 (OD04339) | | 064008 | |
| Kidney Margin | 0.0 | Ovarian Cancer | 0.0 |
| (OD04339) | | (OD04768-07) | |
| Kidney Ca, Clear cell | 0.0 | Ovary Margin | 0.0 |
| type (OD04340) | | (OD04768-08) | |
| Kidney Margin | 0.0 | Normal Stomach | 0.0 |
| (OD04340) | | | |
| Kidney Ca, Nuclear grade | 0.0 | Gastric Cancer | 0.0 |
| 3 (OD04348) | | 9060358 | |
| Kidney Margin | 0.0 | Stomach Margin | 0.0 |
| (OD04348) | | 9060359 | |
| Kidney Cancer | 0.0 | Gastric Cancer | 0.0 |
| (OD04622-01) | | 9060395 | |
| Kidney Margin | 0.0 | Stomach Margin | 0.0 |
| (OD04622-03) | | 9060394 | |
| Kidney Cancer | 0.0 | Gastric Cancer | 0.0 |
| (OD04450-01) | | 9060397 | |
| Kidney Margin | 0.0 | Stomach Margin | 0.0 |
| (OD04450-03) | | 9060396 | |
| Kidney Cancer 8120607 | 0.0 | Gastric Cancer | 0.6 |
| | | 064005 | |

TABLE BKF
Panel 4D

| | Rel. Exp. (%) | | Rel. Exp. (%) |
|---|---|---|---|
| | Ag2460, Run | | Ag2460, Run |
| Tissue Name | 157914794 | Tissue Name | 157914794 |
| Secondary Th1 act | 0.4 | HUVEC IL-1beta | 0.0 |
| Secondary Th2 act | 0.0 | HUVEC IFN gamma | 0.0 |
| Secondary Tr1 act | 0.5 | HUVEC TNF alpha + IFN | 0.0 |
| | | gamma | |
| Secondary Th1 rest | 2.3 | HUVEC TNF alpha + IL4 | 0.0 |
| Secondary Th2 rest | 0.5 | HUVEC IL-11 | 0.0 |
| Secondary Tr1 rest | 3.7 | Lung Microvascular EC | 0.0 |
| | | none | |
| Primary Th1 act | 0.6 | Lung Microvascular EC | 0.0 |
| | | TNF alpha + IL-1beta | |
| Primary Th2 act | 1.1 | Microvascular Dermal EC | 0.0 |
| | | none | |
| Primary Tr1 act | 1.0 | Microsvasular Dermal EC | 0.0 |
| | | TNF alpha + IL-1beta | |
| Primary Th1 rest | 15.0 | Bronchial epithelium | 0.0 |
| | | TNF alpha + IL1beta | |
| Primary Th2 rest | 9.2 | Small airway epithelium | 2.1 |
| | | none | |
| Primary Tr1 rest | 4.3 | Small airway epithelium | 100.0 |
| | | TNF alpha + IL-1beta | |
| CD45RA CD4 | 0.9 | Coronery artery SMC rest | 0.0 |
| lymphocyte act | | | |
| CD45RO CD4 | 1.0 | Coronery artery SMC | 0.0 |
| lymphocyte act | | TNF alpha + IL-1beta | |
| CD8 lymphocyte act | 1.1 | Astrocytes rest | 0.0 |
| Secondary CD8 | 0.5 | Astrocytes TNF alpha + | 0.0 |
| lymphocyte rest | | IL-1beta | |
| Secondary CD8 | 0.5 | KU-812 (Basophil) rest | 0.0 |
| lymphocyte act | | | |
| CD4 lymphocyte none | 1.0 | KU-812 (Basophil) | 0.0 |
| | | PMA/ionomycin | |
| 2ry Th1/Th2/Tr1_anti- | 2.6 | CCD1106 (Keratinocytes) | 0.0 |
| CD95 CH11 | | none | |
| LAK cells rest | 0.7 | CCD1106 (Keratinocytes) | 0.5 |
| | | TNF alpha + IL-1beta | |
| LAK cells IL-2 | 0.0 | Liver cirrhosis | 0.0 |
| LAK cells IL-2 + IL-12 | 0.0 | Lupus kidney | 0.0 |
| LAK cells IL-2 + IFN | 0.5 | NCI-H292 none | 0.3 |
| gamma | | | |
| LAK cells IL-2 + IL-18 | 0.0 | NCI-H292 IL-4 | 0.6 |
| LAK cells | 0.0 | NCI-H292 IL-9 | 1.1 |
| PMA/ionomycin | | | |
| NK Cells IL-2 rest | 0.0 | NCI-H292 IL-13 | 0.0 |
| Two Way MLR 3 day | 0.0 | NCI-H292 IFN gamma | 0.0 |
| Two Way MLR 5 day | 0.0 | HPAEC none | 0.0 |
| Two Way MLR 7 day | 0.8 | HPAEC TNF alpha + IL-1 | 0.0 |
| | | beta | |
| PBMC rest | 2.1 | Lung fibroblast none | 0.0 |
| PBMC PWM | 0.0 | Lung fibroblast TNF | 0.0 |
| | | alpha + IL-1beta | |
| PBMC PHA-L | 2.0 | Lung fibroblast IL-4 | 0.0 |
| Ramos (B cell) none | 0.0 | Lung fibroblast IL-9 | 0.0 |
| Ramos (B cell) | 0.0 | Lung fibroblast IL-13 | 0.0 |
| ionomycin | | | |
| B lymphocytes PWM | 1.8 | Lung fibroblast IFN | 0.0 |
| | | gamma | |
| B lymphocytes CD40L | 1.6 | Dermal fibroblast | 0.0 |
| and IL-4 | | CCD1070 rest | |
| EOL-1 dbcAMP | 0.0 | Dermal fibroblast | 0.8 |
| | | CCD1070 TNF alpha | |
| EOL-1 dbcAMP | 0.0 | Dermal fibroblast | 0.0 |
| PMA/ionomycin | | CCD1070 IL-1beta | |
| Dendritic cells none | 0.0 | Dermal fibroblast IFN | 0.0 |
| | | gamma | |
| Dendritic cells LPS | 0.0 | Dermal fibroblast IL-4 | 0.0 |
| Dendritic cells anti- | 0.0 | IBD Colitis 2 | 0.0 |
| CD40 | | | |
| Monocytes rest | 0.0 | IBD Crohn's | 0.0 |
| Monocytes LPS | 1.1 | Colon | 0.0 |
| Macrophages rest | 0.6 | Lung | 0.0 |
| Macrophages LPS | 0.0 | Thymus | 0.0 |
| HUVEC none | 0.0 | Kidney | 9.9 |
| HUVEC starved | 0.0 | | |

CNS_neurodegeneration_v1.0 Summary: Ag2460 Expression of the NOV69a gene is low/undetectable in all samples on this panel (CTs>35). (Data not shown).

Panel 1 Summary: Ag349 Highest expression of the NOV69a gene is seen in a melanoma cell line (CT=28.7). Thus, expression of this gene could be used to differentiate between this sample and other samples on this panel. There is also significant expression in thymus. Please see Panel 4D for discussion of utility of this gene in autoimmunity.

Panel 1.3D Summary: Ag2460 Expression of the NOV69a gene is limited to a few samples that are all derived from normal tissue. Significant levels of expression are seen in mammary gland, trachea, stomach, thymus, and spinal cord. Thus, expression of this gene can be used to differentiate between these samples and other samples on this panel.

Panel 2D Summary: Ag2460 Expression of the NOV69a gene is limited to a few samples, with highest expression in a lung cancer (CT=27.5). Thus, expression of this gene could be used to differentiate between this sample and other samples on this panel and as a marker to detect the presence of lung cancer. Furthermore, therapeutic modulation of the expression or function of this gene may be effective in the treatment of lung cancer.

Panel 4D Summary: Ag2460 The NOV69a gene encodes a homolog of the IL-1 epsilon. Interleukin 1 (IL-1) is a member of a large family of cytokines, which modulates immune and inflammatory responses. IL-1 molecules such as IL-1alpha, -beta, -delta, -gamma, and IL1-receptor agonist (IL-1ra) are typically secreted by macrophages, mononuclear cells, epithelial and endothelial cells. IL-1 molecules are first produced as precursors of about 30 kDa and do not contain a signal sequence. The IL-1 precursors are then proteolytically cleaved into their secreted active forms (˜17 kDa). Their immuno-modulatory functions are mediated by two IL-1 receptors, which are members of the immunoglobulin superfamily. The biological functions of IL-1 include: activation of vascular endothelial cells to secrete IL-6, increase leukocyte adhesion and activate mononuclear phagocytes that activate inflammatory leukocytes; tissue destruction, and fever. Given the biological potency of the IL-1 family of proteins, a need exists to identify new members of this family as well as understand the biological function of its members. The high levels of expression of this gene in small airway epithelium activated by treatment with TNF-alpha+IL-1 beta(CT=28.9) indicate that CG56136-01 may play a substantial role in mediating inflammation in the lung. Thus, therapeutic targeting of CG56136-01 with a monoclonal antibody is anticipated to limit or block the extent of inflammation potential and thus the symptoms, caused by pro-inflammatory cytokines such as IL-1 epsilon, when these cytokines are induced in allergic, asthma and COPD patients.

References:

Smith, D. E., Renshaw, B. R., Ketchem, R. R., Kubin, M., Garka, K. E. and Sims, J. E. Four new members expand the interleukin-1 superfamily J. Biol. Chem. 275 (2), 1169-1175 (2000)

Abstract: We report here the cloning and characterization of four new members of the interleukin-1 (IL-1) family (FIL1delta, FIL1epsilon, FIL1zeta, and FIL1eta, with FIL1 standing for “Family of IL-1”). The novel genes demonstrate significant sequence similarity to IL-1alpha, IL-1beta, IL-1ra, and IL-18, and in addition maintain a conserved exon-intron arrangement that is shared with the previously known members of the family. Protein structure modeling also suggests that the FIL1 genes are related to IL-1beta and IL-1ra. The novel genes form a cluster with the IL-1s on the long arm of human chromosome 2.

NOV71

Expression of gene NOV71 was assessed using the primer-probe set Ag3049, described in Table BLA. Results of the RTQ-PCR runs are shown in Tables BLB, BLC, BLD and BLE.

TABLE BLA
Probe Name Ag3049

| | | | Start | |
|---|---|---|---|---|
| Primers | Sequences | Length | Position | SEQ ID NO: |
| Forward | 5′-gctggcgatatttaatgaattg-3′ | 22 | 696 | 1279 |
| Probe | TET-5′-catgcagacgtggatctttacgcact-3′-TAMRA | 26 | 718 | 1280 |
| Reverse | 5′-agtacaatggcaacagcatcat-3′ | 22 | 767 | 1281 |

TABLE BLB
CNS_neurodegeneration_v1.0

| | | | Rel. Exp. |
|---|---|---|---|
| | Rel. Exp. (%) | | (%) Ag3049, |
| | Ag3049, Run | | Run |
| Tissue Name | 209823735 | Tissue Name | 209823735 |
| AD 1 Hippo | 10.2 | Control (Path) 3 | 5.9 |
| | | Temporal Ctx | |
| AD 2 Hippo | 33.7 | Control (Path) 4 | 28.1 |
| | | Temporal Ctx | |
| AD 3 Hippo | 9.3 | AD 1 Occipital Ctx | 14.2 |
| AD 4 Hippo | 7.7 | AD 2 Occipital Ctx | 0.0 |
| | | (Missing) | |
| AD 5 hippo | 99.3 | AD 3 Occipital Ctx | 5.8 |
| AD 6 Hippo | 46.3 | AD 4 Occipital Ctx | 16.0 |
| Control 2 Hippo | 44.1 | AD 5 Occipital Ctx | 17.1 |
| Control 4 Hippo | 6.0 | AD 6 Occipital Ctx | 63.7 |
| Control (Path) 3 | 7.6 | Control 1 Occipital | 2.2 |
| Hippo | | Ctx | |
| AD 1 Temporal Ctx | 13.0 | Control 2 Occipital | 68.3 |
| | | Ctx | |
| AD 2 Temporal Ctx | 33.4 | Control 3 Occipital | 11.5 |
| | | Ctx | |
| AD 3 Temporal Ctx | 6.8 | Control 4 Occipital | 5.5 |
| | | Ctx | |
| AD 4 Temporal Ctx | 16.5 | Control (Path) 1 | 77.9 |
| | | Occipital Ctx | |
| AD 5 Inf Temporal | 100.0 | Control (Path) 2 | 8.6 |
| Ctx | | Occipital Ctx | |
| AD 5 SupTemporal | 48.0 | Control (Path) 3 | 2.1 |
| Ctx | | Occipital Ctx | |
| AD 6 Inf Temporal | 37.9 | Control (Path) 4 | 12.9 |
| Ctx | | Occipital Ctx | |
| AD 6 Sup Temporal | 41.5 | Control 1 Parietal | 4.1 |
| Ctx | | Ctx | |
| Control 1 Temporal | 6.3 | Control 2 Parietal | 29.9 |
| Ctx | | Ctx | |
| Control 2 Temporal | 62.4 | Control 3 Parietal | 18.0 |
| Ctx | | Ctx | |
| Control 3 Temporal | 14.2 | Control (Path) 1 | 97.9 |
| Ctx | | Parietal Ctx | |
| Control 4 Temporal | 7.7 | Control (Path) 2 | 19.3 |
| Ctx | | Parietal Ctx | |
| Control (Path) 1 | 64.6 | Control (Path) 3 | 6.3 |
| Temporal Ctx | | Parietal Ctx | |
| Control (Path) 2 | 29.9 | Control (Path) 4 | 42.6 |
| Temporal Ctx | | Parietal Ctx | |

TABLE BLC
Panel 1.3D

| | Rel. Exp. (%) Ag3049, | | Rel. Exp. (%) Ag3049, |
|---|---|---|---|
| Tissue Name | Run 167972763 | Tissue Name | Run 167972763 |
| Liver adenocarcinoma | 37.6 | Kidney (fetal) | 7.5 |
| Pancreas | 1.8 | Renal ca. 786-0 | 4.7 |
| Pancreatic ca. CAPAN 2 | 8.0 | Renal ca. A498 | 8.7 |
| Adrenal gland | 2.1 | Renal ca. RXF 393 | 28.3 |
| Thyroid | 3.0 | Renal ca. ACHN | 6.3 |
| Salivary gland | 1.5 | Renal ca. UO-31 | 36.9 |
| Pituitary gland | 8.5 | Renal ca. TK-10 | 9.5 |
| Brain (fetal) | 10.4 | Liver | 0.2 |
| Brain (whole) | 9.0 | Liver (fetal) | 1.7 |
| Brain (amygdala) | 18.7 | Liver ca. | 6.3 |
| | | (hepatoblast) HepG2 | |
| Brain (cerebellum) | 32.5 | Lung | 0.4 |
| Brain (hippocampus) | 14.0 | Lung (fetal) | 2.0 |
| Brain (substantia nigra) | 6.0 | Lung ca. (small cell) | 5.4 |
| | | LX-1 | |
| Brain (thalamus) | 2.9 | Lung ca. (small cell) | 3.6 |
| | | NCI-H69 | |
| Cerebral Cortex | 30.1 | Lung ca. (s.cell var.) | 15.9 |
| | | SHP-77 | |
| Spinal cord | 3.4 | Lung ca. (large | 1.7 |
| | | cell)NCI-H460 | |
| glio/astro U87-MG | 27.4 | Lung ca. (non-sm. | 28.9 |
| | | cell) A549 | |
| glio/astro U-118-MG | 16.0 | Lung ca. (non-s.cell) | 19.6 |
| | | NCI-H23 | |
| astrocytoma SW1783 | 40.3 | Lung ca. (non-s.cell) | 18.3 |
| | | HOP-62 | |
| neuro*; met SK-N-AS | 0.6 | Lung ca. (non-s.cl) | 23.5 |
| | | NCI-H522 | |
| astrocytoma SF-539 | 2.9 | Lung ca. (squam.) | 15.4 |
| | | SW 900 | |
| astrocytoma SNB-75 | 26.8 | Lung ca. (squam.) | 4.0 |
| | | NCI-H596 | |
| glioma SNB-19 | 34.4 | Mammary gland | 1.1 |
| glioma U251 | 100.0 | Breast ca.* (pl.ef) | 37.4 |
| | | MCF-7 | |
| glioma SF-295 | 39.2 | Breast ca.* (pl.ef) | 25.7 |
| | | MDA-MB-231 | |
| Heart (fetal) | 1.3 | Breast ca.* (pl.ef) | 55.5 |
| | | T47D | |
| Heart | 1.2 | Breast ca. BT-549 | 7.4 |
| Skeletal muscle (fetal) | 2.0 | Breast ca. MDA-N | 2.0 |
| Skeletal muscle | 9.3 | Ovary | 2.7 |
| Bone marrow | 0.3 | Ovarian ca. OVCAR-3 | 2.6 |
| Thymus | 3.0 | Ovarian ca. OVCAR-4 | 17.4 |
| Spleen | 2.2 | Ovarian ca. OVCAR-5 | 50.7 |
| Lymph node | 5.0 | Ovarian ca. OVCAR-8 | 1.1 |
| Colorectal | 1.9 | Ovarian ca. IGROV-1 | 5.2 |
| Stomach | 3.3 | Ovarian ca.* (ascites) | 24.7 |
| | | SK-OV-3 | |
| Small intestine | 0.2 | Uterus | 0.1 |
| Colon ca. SW480 | 7.9 | Placenta | 0.2 |
| Colon ca.* | 30.4 | Prostate | 3.7 |
| SW620(SW480 met) | | | |
| Colon ca. HT29 | 8.7 | Prostate ca.* (bone | 91.4 |
| | | met)PC-3 | |
| Colon ca. HCT-116 | 12.4 | Testis | 0.5 |
| Colon ca. CaCo-2 | 18.7 | Melanoma | 6.0 |
| | | Hs688(A).T | |
| Colon ca. | 3.7 | Melanoma* (met) | 10.1 |
| tissue(ODO3866) | | Hs688(B).T | |
| Colon ca. HCC-2998 | 8.9 | Melanoma UACC-62 | 11.0 |
| Gastric ca.* (liver met) | 8.7 | Melanoma M14 | 0.4 |
| NCI-N87 | | | |
| Bladder | 10.7 | Melanoma LOX | 17.8 |
| | | IMVI | |
| Trachea | 1.0 | Melanoma* (met) | 1.7 |
| | | SK-MEL-5 | |
| Kidney | 2.2 | Adipose | 3.9 |

TABLE BLD
Panel 2.2

| | Rel. Exp. (%) Ag3049, Run | | Rel. Exp. (%) Ag3049, Run |
|---|---|---|---|
| Tissue Name | 174441445 | Tissue Name | 174441445 |
| Normal Colon | 11.7 | Kidney Margin | 64.6 |
| | | (OD04348) | |
| Colon cancer | 57.4 | Kidney malignant | 10.5 |
| (OD06064) | | cancer (OD06204B) | |
| Colon Margin | 3.4 | Kidney normal adjacent | 10.1 |
| (OD06064) | | tissue (OD06204E) | |
| Colon cancer | 12.1 | Kidney Cancer | 24.1 |
| (OD06159) | | (OD04450-01) | |
| Colon Margin | 1.8 | Kidney Margin | 21.6 |
| (OD06159) | | (OD04450-03) | |
| Colon cancer | 7.1 | Kidney Cancer 8120613 | 9.9 |
| (OD06297-04) | | | |
| Colon Margin | 14.7 | Kidney Margin | 13.6 |
| (OD06297-015) | | 8120614 | |
| CC Gr.2 ascend colon | 5.1 | Kidney Cancer 9010320 | 10.3 |
| (ODO3921) | | | |
| CC Margin (ODO3921) | 5.4 | Kidney Margin | 11.3 |
| | | 9010321 | |
| Colon cancer metastasis | 1.8 | Kidney Cancer 8120607 | 30.6 |
| (OD06104) | | | |
| Lung Margin | 14.8 | Kidney Margin | 11.2 |
| (OD06104) | | 8120608 | |
| Colon mets to lung | 25.5 | Normal Uterus | 2.0 |
| (OD04451-01) | | | |
| Lung Margin | 51.4 | Uterine Cancer 064011 | 6.8 |
| (OD04451-02) | | | |
| Normal Prostate | 28.9 | Normal Thyroid | 6.3 |
| Prostate Cancer | 13.4 | Thyroid Cancer 064010 | 14.2 |
| (OD04410) | | | |
| Prostate Margin | 20.3 | Thyroid Cancer | 51.4 |
| (OD04410) | | A302152 | |
| Normal Ovary | 7.9 | Thyroid Margin | 8.5 |
| | | A302153 | |
| Ovarian cancer | 56.3 | Normal Breast | 53.6 |
| (OD06283-03) | | | |
| Ovarian Margin | 13.6 | Breast Cancer | 6.9 |
| (OD06283-07) | | (OD04566) | |
| Ovarian Cancer 064008 | 24.1 | Breast Cancer 1024 | 100.0 |
| Ovarian cancer | 31.2 | Breast Cancer | 9.5 |
| (OD06145) | | (OD04590-01) | |
| Ovarian Margin | 14.0 | Breast Cancer Mets | 24.3 |
| (OD06145) | | (OD04590-03) | |
| Ovarian cancer | 10.4 | Breast Cancer | 65.1 |
| (OD06455-03) | | Metastasis (OD04655- | |
| | | 05) | |
| Ovarian Margin | 1.0 | Breast Cancer 064006 | 26.2 |
| (OD06455-07) | | | |
| Normal Lung | 11.8 | Breast Cancer 9100266 | 24.5 |
| Invasive poor diff. lung | 10.7 | Breast Margin 9100265 | 25.0 |
| adeno (ODO4945-01 | | | |
| Lung Margin | 6.2 | Breast Cancer A209073 | 19.6 |
| (ODO4945-03) | | | |
| Lung Malignant Cancer | 5.9 | Breast Margin | 58.6 |
| (OD03126) | | A2090734 | |
| Lung Margin | 3.3 | Breast cancer | 79.0 |
| (OD03126) | | (OD06083) | |
| Lung Cancer | 42.3 | Breast cancer node | 40.1 |
| (OD05014A) | | metastasis (OD06083) | |
| Lung Margin | 13.0 | Normal Liver | 31.0 |
| (OD05014B) | | | |
| Lung cancer (OD06081) | 13.1 | Liver Cancer 1026 | 12.5 |
| Lung Margin | 8.1 | Liver Cancer 1025 | 25.5 |
| (OD06081) | | | |
| Lung Cancer | 35.8 | Liver Cancer 6004-T | 14.8 |
| (OD04237-01) | | | |
| Lung Margin | 14.7 | Liver Tissue 6004-N | 4.7 |
| (OD04237-02) | | | |
| Ocular Melanoma | 0.0 | Liver Cancer 6005-T | 25.9 |
| Metastasis | | | |
| Ocular Melanoma | 8.5 | Liver Tissue 6005-N | 70.7 |
| Margin (Liver) | | | |
| Melanoma Metastasis | 4.3 | Liver Cancer 064003 | 14.5 |
| Melanoma Margin | 6.5 | Normal Bladder | 32.3 |
| (Lung) | | | |
| Normal Kidney | 16.5 | Bladder Cancer 1023 | 6.6 |
| Kidney Ca, Nuclear | 41.2 | Bladder Cancer | 51.1 |
| grade 2 (OD04338) | | A302173 | |
| Kidney Margin | 10.8 | Normal Stomach | 72.7 |
| (OD04338) | | | |
| Kidney Ca Nuclear | 45.7 | Gastric Cancer 9060397 | 4.7 |
| grade 1/2 (OD04339) | | | |
| Kidney Margin | 8.2 | Stomach Margin | 40.6 |
| (OD04339) | | 9060396 | |
| Kidney Ca, Clear cell | 18.4 | Gastric Cancer 9060395 | 10.4 |
| type (OD04340) | | | |
| Kidney Margin | 7.4 | Stomach Margin | 57.8 |
| (OD04340) | | 9060394 | |
| Kidney Ca, Nuclear | 17.9 | Gastric Cancer 064005 | 24.7 |
| grade 3 (OD04348) | | | |

TABLE BLE
Panel 4D

| | Rel. Exp. (%) | | Rel. Exp. (%) |
|---|---|---|---|
| | Ag3049, Run | | Ag3049, Run |
| Tissue Name | 164334396 | Tissue Name | 164334396 |
| Secondary Th1 act | 9.4 | HUVEC IL-1 beta | 1.0 |
| Secondary Th2 act | 7.8 | HUVEC IFN gamma | 3.1 |
| Secondary Tr1 act | 8.1 | HUVEC TNF alpha + IFN | 2.1 |
| | | gamma | |
| Secondary Th1 rest | 0.3 | HUVEC TNF alpha + IL4 | 1.0 |
| Secondary Th2 rest | 2.0 | HUVEC IL-11 | 0.7 |
| Secondary Tr1 rest | 2.1 | Lung Microvascular EC | 0.3 |
| | | none | |
| Primary Th1 act | 13.0 | Lung Microvascular EC | 0.2 |
| | | TNF alpha + IL-1 beta | |
| Primary Th2 act | 13.5 | Microvascular Dermal EC | 0.5 |
| | | none | |
| Primary Tr1 act | 16.2 | Microsvasular Dermal EC | 0.1 |
| | | TNF alpha + IL-1 beta | |
| Primary Th1 rest | 3.8 | Bronchial epithelium | 4.5 |
| | | TNF alpha + IL1 beta | |
| Primary Th2 rest | 3.7 | Small airway epithelium | 2.6 |
| | | none | |
| Primary Tr1 rest | 1.0 | Small airway epithelium | 8.5 |
| | | TNF alpha + IL-1 beta | |
| CD45RA CD4 | 6.6 | Coronery artery SMC rest | 11.8 |
| lymphocyte act | | | |
| CD45RO CD4 | 7.8 | Coronery artery SMC | 6.8 |
| lymphocyte act | | TNF alpha + IL-1 beta | |
| CD8 lymphocyte act | 5.6 | Astrocytes rest | 3.8 |
| Secondary CD8 | 4.0 | Astrocytes TNF alpha + | 5.4 |
| lymphocyte rest | | IL-1 beta | |
| Secondary CD8 | 9.2 | KU-812 (Basophil) rest | 1.5 |
| lymphocyte act | | | |
| CD4 lymphocyte none | 0.6 | KU-812 (Basophil) | 10.4 |
| | | PMA/ionomycin | |
| 2ry Th1/Th2/Tr1_anti- | 3.7 | CCD1106 (Keratinocytes) | 3.7 |
| CD95 CH11 | | none | |
| LAK cells rest | 9.3 | CCD1106 (Keratinocytes) | 4.5 |
| | | TNF alpha + IL-1 beta | |
| LAK cells IL-2 | 3.0 | Liver cirrhosis | 0.5 |
| LAK cells IL-2 + IL-12 | 4.6 | Lupus kidney | 0.3 |
| LAK cells IL-2 + IFN | 9.6 | NCI-H292 none | 9.5 |
| gamma | | | |
| LAK cells IL-2 + IL-18 | 9.5 | NCI-H292 IL-4 | 15.1 |
| LAK cells | 4.7 | NCI-H292 IL-9 | 12.5 |
| PMA/ionomycin | | | |
| NK Cells IL-2 rest | 1.4 | NCI-H292 IL-13 | 7.7 |
| Two Way MLR 3 day | 3.6 | NCI-H292 IFN gamma | 9.0 |
| Two Way MLR 5 day | 3.7 | HPAEC none | 2.1 |
| Two Way MLR 7 day | 2.3 | HPAEC TNF alpha + IL-1 | 1.1 |
| | | beta | |
| PBMC rest | 1.1 | Lung fibroblast none | 2.5 |
| PBMC PWM | 17.6 | Lung fibroblast TNF alpha + | 1.2 |
| | | IL-1 beta | |
| PBMC PHA-L | 10.8 | Lung fibroblast IL-4 | 5.0 |
| Ramos (B cell) none | 23.2 | Lung fibroblast IL-9 | 7.5 |
| Ramos (B cell) | 100.0 | Lung fibroblast IL-13 | 3.9 |
| ionomycin | | | |
| B lymphocytes PWM | 40.9 | Lung fibroblast IFN | 14.6 |
| | | gamma | |
| B lymphocytes CD40L | 25.2 | Dermal fibroblast | 9.4 |
| and IL-4 | | CCD1070 rest | |
| EOL-1 dbcAMP | 0.0 | Dermal fibroblast | 14.8 |
| | | CCD1070 TNF alpha | |
| EOL-1 dbcAMP | 0.4 | Dermal fibroblast | 6.0 |
| PMA/ionomycin | | CCD1070 IL-1 beta | |
| Dendritic cells none | 7.7 | Dermal fibroblast IFN | 10.2 |
| | | gamma | |
| Dendritic cells LPS | 10.8 | Dermal fibroblast IL-4 | 9.2 |
| Dendritic cells anti- | 13.1 | IBD Colitis 2 | 0.7 |
| CD40 | | | |
| Monocytes rest | 0.9 | IBD Crohn's | 0.1 |
| Monocytes LPS | 0.3 | Colon | 1.1 |
| Macrophages rest | 19.9 | Lung | 1.1 |
| Macrophages LPS | 4.3 | Thymus | 3.7 |
| HUVEC none | 2.7 | Kidney | 7.4 |
| HUVEC starved | 2.9 | | |

CNS_neurodegeneration_v1.0 Summary: Ag3049 This panel does not show differential expression of the NOV71 gene in Alzheimer's disease. However, this expression profile confirms the presence of this gene in the brain. Please see Panel 1.3D for discussion of utility of this gene in the central nervous system.

Panel 1.3D Summary: Ag3049 The NOV71 gene is expressed at moderate to high leves in the cancer cell lines in this panel with the highest expression shown by a glioma cell line U251 (CT=26.6). Because normal tissues show a lower level of expression of this gene, expression of this gene migh be used as a diagnostic marker for cancer. Furthermore, therapeutics designed using antibodies and small molecule inhibitors of this gene may be effective in the treatment of cancer.

Among tissues with metabolic function, this gene has moderate levels of expression in pancreas, adrenal, thyroid, pituitary, heart, skeletal muscle and adipose. Therefore, this gene product may be a small molecule target for the treatment of endocrine and metabolic diseases, including obesity, and Types 1 and 2 diabetes.

In addition, moderate expression of this gene in the CNS suggests a role for this gene product in brain processes. Inhibition of SODIUM/HYDROGEN EXCHANGER function in the brain is associated with the activity of several enzymes known to play a positive role in cell survival and learning and memory, such as PKA and PKC. Therefore, inhibitors of the protein encoded by this gene may have utility in mimicking the potentially therapeutic action of these enzymes in the treatment of neurodegenerative diseases including Alzheimer's and Parkinson's diseases, as well as in memory loss due to aging.

References:

Am J Physiol Cell Physiol October 2001;281(4):C1146-Acute inhibition of brain-specific Na(+)/H(+) exchanger isoform 5 by protein kinases A and C and cell shrinkage. Attaphitaya S, Nehrke K, Melvin J E.

Little is known of the functional properties of the mammalian, brain-specific Na(+)/H(+) exchanger isoform 5 (NHE5). Rat NHE5 was stably expressed in NHE-deficient PS120 cells, and its activity was characterized using the fluorescent pH-sensitive dye 2′,7′-bis(2-carboxyethyl)-5(6)-carboxyfluorescein. NHE5 was insensitive to ethylisopropyl amiloride. The transport kinetics displayed a simple Michaelis-Menten relationship for extracellular Na(+) (apparent K(Na)=27+/−5 mM) and a Hill coefficient near 3 for the intracellular proton concentration with a half-maximal activity at an intracellular pH of 6.93+/−0.03. NHE5 activity was inhibited by acute exposure to 8-bromo-cAMP or forskolin (which increases intracellular cAMP by activating adenylate cyclase). The kinase inhibitor H-89 reversed this inhibition, suggesting that regulation by cAMP involves a protein kinase A (PKA)-dependent process. In contrast, 8-bromo-cGMP did not have a significant effect on activity. The protein kinase C (PKC) activator phorbol 12-myristrate 13-acetate inhibited, NHE5, and the PKC antagonist chelerythrine chloride blunted this effect. Activity was also inhibited by hyperosmotic-induced cell shrinkage but was unaffected by a hyposmotic challenge. These results demonstrate that rat brain NHE5 is downregulated by activation of PKA and PKC and by cell shrinkage, important regulators of neuronal cell function.

Panel 2.2 Summary: Ag3049 The NOV71 gene can be used as a diagnostic marker for stomach, breast, lung, ovarian and some colon cancers as expression in the normal adjacent tissue and the tumor tissue differs. Antibodies and small molecule inhibitors designed with this gene product may also be used for therapy in breast, lung, ovarian and some colon cancers.

Panel 4D Summary: Ag3049 The NOV71 gene, a sodium/hydrogen Exchanger homolog is expressed at a high level in Ramos (B cell) activated with ionomycin (CT=24.72), and at a moderate to high level in other activated B cell preparations. Therefore, small molecule antagonists or therapeutic antibody antagonists that block the function of the NOV71 gene product may be useful in several autoimmune and inflammatory diseases in which activated B cells can play major roles as sources of autoantibody-producing cells and also as powerful antigen-presenting cells, including, but not limited to, Crohn's disease, ulcerative colitis, multiple sclerosis, chronic obstructive pulmonary disease, asthma, emphysema, rheumatoid arthritis, lupus erythematosus, or psoriasis.

NOV72: Ubiquitin-Specific Protease

Expression of gene NOV72 was assessed using the primer-probe set Ag3050, described in Table BMA. Results of the RTQ-PCR runs are shown in Tables BMB, BMC, BMD and BME.

TABLE BMA
Probe Name Ag3050

| | | | Start | |
|---|---|---|---|---|
| Primers | Seqences | Length | Position | SEQ ID NO: |
| Forward | 5′-tgaccaggtattaaccatggaa-3′ | 22 | 799 | 1282 |
| Probe | TET-5′-ttactgctgcagggacatgctctcct-3′-TAMRA | 26 | 823 | 1283 |
| Reverse | 5′-taggcaaaggtctctttgtcaa-3′ | 22 | 852 | 1284 |

TABLE BMB
CNS_neurodegeneration_v1.0

| | Rel. Exp. (%) Ag3050, | | Rel. Exp. (%) Ag3050, |
|---|---|---|---|
| Tissue Name | Run 211012446 | Tissue Name | Run 211012446 |
| AD 1 Hippo | 12.5 | Control (Path) 3 | 4.3 |
| | | Temporal Ctx | |
| AD 2 Hippo | 22.1 | Control (Path) 4 | 29.1 |
| | | Temporal Ctx | |
| AD 3 Hippo | 3.0 | AD 1 Occipital Ctx | 15.0 |
| AD 4 Hippo | 11.9 | AD 2 Occipital Ctx | 0.0 |
| | | (Missing) | |
| AD 5 Hippo | 59.0 | AD 3 Occipital Ctx | 7.1 |
| AD 6 Hippo | 51.1 | AD 4 Occipital Ctx | 22.2 |
| Control 2 Hippo | 17.6 | AD 5 Occipital Ctx | 24.7 |
| Control 4 Hippo | 6.1 | AD 6 Occipital Ctx | 22.4 |
| Control (Path) 3 | 7.0 | Control 1 Occipital | 2.9 |
| Hippo | | Ctx | |
| AD 1 Temporal Ctx | 17.4 | Control 2 Occipital | 39.0 |
| | | Ctx | |
| AD 2 Temporal Ctx | 23.5 | Control 3 Occipital | 16.8 |
| | | Ctx | |
| AD 3 Temporal Ctx | 7.9 | Control 4 Occipital | 6.0 |
| | | Ctx | |
| AD 4 Temporal Ctx | 27.5 | Control (Path) 1 | 51.4 |
| | | Occipital Ctx | |
| AD 5 Inf Temporal | 100.0 | Control (Path) 2 | 12.2 |
| Ctx | | Occipital Ctx | |
| AD 5 Sup Temporal | 34.9 | Control (Path) 3 | 2.4 |
| Ctx | | Occipital Ctx | |
| AD 6 Inf Temporal | 54.7 | Control (Path) 4 | 15.9 |
| Ctx | | Occipital Ctx | |
| AD 6 Sup Temporal | 60.3 | Control 1 Parietal | 8.4 |
| Ctx | | Ctx | |
| Control 1 Temporal | 3.7 | Control 2 Parietal | 36.3 |
| Ctx | | Ctx | |
| Control 2 Temporal | 20.3 | Control 3 Parietal | 15.9 |
| Ctx | | Ctx | |
| Control 3 Temporal | 9.7 | Control (Path) 1 | 39.0 |
| Ctx | | Parietal Ctx | |
| Control 3 Temporal | 7.2 | Control (Path) 2 | 16.2 |
| Ctx | | Parietal Ctx | |
| Control (Path) 1 | 46.7 | Control (Path) 3 | 2.2 |
| Temporal Ctx | | Parietal Ctx | |
| Control (Path) 2 | 34.9 | Control (Path) 4 | 36.1 |
| Temporal Ctx | | Parietal Ctx | |

TABLE BMC
Panel 1.3D

| | Rel. Exp. (%) Ag3050, | | Rel. Exp. (%) Ag3050, |
|---|---|---|---|
| Tissue Name | Run 167985384 | Tissue Name | Run 167985384 |
| Liver adenocarcinoma | 10.1 | Kidney (fetal) | 28.7 |
| Pancreas | 5.7 | Renal ca. 786-0 | 14.9 |
| Pancreatic ca. CAPAN 2 | 2.5 | Renal ca. A498 | 6.6 |
| Adrenal gland | 2.5 | Renal ca. RXF 393 | 8.8 |
| Thyroid | 3.7 | Renal ca. ACHN | 5.4 |
| Salivary gland | 1.5 | Renal ca. UO-31 | 6.2 |
| Pituitary gland | 6.8 | Renal ca. TK-10 | 6.5 |
| Brain (fetal) | 100.0 | Liver | 2.2 |
| Brain (whole) | 23.3 | Liver (fetal) | 6.4 |
| Brain (amygdala) | 22.2 | Liver ca. | 11.3 |
| | | (hepatoblast) HepG2 | |
| Brain (cerebellum) | 88.9 | Lung | 0.8 |
| Brain (hippocampus) | 19.5 | Lung (fetal) | 15.1 |
| Brain (substantia nigra) | 19.3 | Lung ca. (small cell) | 14.4 |
| | | LX-1 | |
| Brain (thalamus) | 7.7 | Lung ca. (small cell) | 18.4 |
| | | NCI-H69 | |
| Cerebral Cortex | 12.1 | Lung ca. (s.cell var.) | 43.2 |
| | | SHP-77 | |
| Spinal cord | 11.1 | Lung ca. (large | 0.0 |
| | | cell)NCI-H460 | |
| glio/astro U87-MG | 4.6 | Lung ca. (non-sm. | 5.2 |
| | | cell) A549 | |
| glio/astro U-118-MG | 18.9 | Lung ca. (non-s.cell) | 7.4 |
| | | NCI-H23 | |
| astrocytoma SW1783 | 9.4 | Lung ca. (non-s.cell) | 7.2 |
| | | HOP-62 | |
| neuro*; met SK-N-AS | 15.7 | Lung ca. (non-s.cl) | 21.0 |
| | | NCI-H522 | |
| astrocytoma SF-539 | 17.1 | Lung ca. (squam.) | 7.7 |
| | | SW 900 | |
| astrocytoma SNB-75 | 12.2 | Lung ca. (squam.) | 60.7 |
| | | NCI-H596 | |
| glioma SNB-19 | 11.9 | Mammary gland | 1.3 |
| glioma U251 | 15.7 | Breast ca.* (pl.ef) | 14.5 |
| | | MCF-7 | |
| glioma SF-295 | 16.4 | Breast ca.* (pl.ef) | 24.7 |
| | | MDA-MB-231 | |
| Heart (fetal) | 0.0 | Breast ca.* (pl.ef) | 21.8 |
| | | T47D | |
| Heart | 3.8 | Breast ca. BT-549 | 7.3 |
| Skeletal muscle (fetal) | 8.1 | Breast ca. MDA-N | 13.5 |
| Skeletal muscle | 11.0 | Ovary | 4.0 |
| Bone marrow | 4.5 | Ovarian ca. OVCAR-3 | 2.2 |
| Thymus | 9.6 | Ovarian ca. OVCAR-4 | 4.7 |
| Spleen | 5.4 | Ovarian ca. OVCAR-5 | 11.7 |
| Lymph node | 14.6 | Ovarian ca. OVCAR-8 | 4.9 |
| Colorectal | 5.6 | Ovarian ca. IGROV-1 | 2.2 |
| Stomach | 2.0 | Ovarian ca* (ascites) | 24.3 |
| | | SK-OV-3 | |
| Small intestine | 1.6 | Uterus | 6.9 |
| Colon ca. SW480 | 5.1 | Placenta | 1.3 |
| Colon ca.* | 22.2 | Prostate | 5.9 |
| SW620(SW480 met) | | | |
| Colon ca. HT29 | 4.9 | Prostate ca.* (bone | 4.8 |
| | | met)PC-3 | |
| Colon ca. HCT-116 | 9.3 | Testis | 11.2 |
| Colon ca. CaCo-2 | 18.2 | Melanoma | 1.7 |
| | | Hs688(A).T | |
| Colon ca. | 2.4 | Melanoma* (met) | 0.0 |
| tissue(ODO3866) | | Hs688(B).T | |
| Colon ca. HCC-2998 | 10.1 | Melanoma UACC-62 | 4.0 |
| Gastric ca.* (liver met) | 9.8 | Melanoma M14 | 2.1 |
| NCI-N87 | | | |
| Bladder | 6.3 | Melanoma LOX | 5.8 |
| | | IMVI | |
| Trachea | 2.2 | Melanoma* (met) | 2.1 |
| | | SK-MEL-5 | |
| Kidney | 6.7 | Adipose | 4.3 |

TABLE BMD
Panel 4D

| | Rel. Exp. (%) | | Rel. Exp. (%) |
|---|---|---|---|
| | Ag3050, Run | | Ag3050, Run |
| Tissue Name | 164317257 | Tissue Name | 164317257 |
| Secondary Th1 act | 21.0 | HUVEC IL-1 beta | 5.3 |
| Secondary Th2 act | 22.4 | HUVEC IFN gamma | 6.3 |
| Secondary Tr1 act | 23.3 | HUVEC TNF alpha + IFN | 11.6 |
| | | gamma | |
| Secondary Th1 rest | 4.0 | HUVEC TNF alpha + IL4 | 7.5 |
| Secondary Th2 rest | 7.9 | HUVEC IL-11 | 2.6 |
| Secondary Tr1 rest | 9.8 | Lung Microvascular EC | 9.5 |
| | | none | |
| Primary Th1 act | 24.3 | Lung Microvascular EC | 9.9 |
| | | TNF alpha + IL-1 beta | |
| Primary Th2 act | 13.5 | Microvascular Dermal EC | 13.4 |
| | | none | |
| Primary Tr1 act | 21.6 | Microsvasular Dermal EC | 11.7 |
| | | TNF alpha + IL-1 beta | |
| Primary Th1 rest | 36.6 | Bronchial epithelium | 21.9 |
| | | TNF alpha + IL1 beta | |
| Primary Th2 rest | 17.6 | Small airway epithelium | 0.0 |
| | | none | |
| Primary Tr1 rest | 19.6 | Small airway epithelium | 15.7 |
| | | TNF alpha + IL-1 beta | |
| CD45RA CD4 | 7.1 | Coronery artery SMC rest | 4.0 |
| lymphocyte act | | | |
| CD45RO CD4 | 14.5 | Coronery artery SMC | 1.4 |
| lymphocyte act | | TNF alpha + IL-1 beta | |
| CD8 lymphocyte act | 7.7 | Astrocytes rest | 11.8 |
| Secondary CD8 | 17.2 | Astrocytes TNF alpha + | 1.6 |
| lymphocyte rest | | IL-1 beta | |
| Secondary CD8 | 13.9 | KU-812 (Basophil) rest | 15.6 |
| lymphocyte act | | | |
| CD4 lymphocyte none | 2.1 | KU-812 (Basophil) | 40.1 |
| | | PMA/ionomycin | |
| 2ry Th1/Th2/Tr1_anti- | 17.0 | CCD1106 (Keratinocytes) | 11.0 |
| CD95 CH11 | | none | |
| LAK cells rest | 16.7 | CCD1106 (Keratinocytes) | 4.6 |
| | | TNF alpha + IL-1 beta | |
| LAK cells IL-2 | 15.1 | Liver cirrhosis | 5.3 |
| LAK cells IL-2 + IL-12 | 12.6 | Lupus kidney | 1.6 |
| LAK cells IL-2 + IFN | 15.3 | NCI-H292 none | 17.1 |
| gamma | | | |
| LAK cells IL-2 + IL-18 | 14.9 | NCI-H292 IL-4 | 15.3 |
| LAK cells | 7.2 | NCI-H292 IL-9 | 15.5 |
| PMA/ionomycin | | | |
| NK Cells IL-2 rest | 14.3 | NCI-H292 IL-13 | 5.2 |
| Two Way MLR 3 day | 7.5 | NCI-H292 IFN gamma | 9.3 |
| Two Way MLR 5 day | 7.0 | HPAEC none | 9.7 |
| Two Way MLR 7 day | 6.0 | HPAEC TNF alpha + IL-1 | 13.9 |
| | | beta | |
| PBMC rest | 3.4 | Lung fibroblast none | 5.3 |
| PBMC PWM | 38.4 | Lung fibroblast TNF alpha + | 5.7 |
| | | IL-1 beta | |
| PBMC PHA-L | 14.5 | Lung fibroblast IL-4 | 7.7 |
| Ramos (B cell) none | 24.8 | Lung fibroblast IL-9 | 7.0 |
| Ramos (B cell) | 48.0 | Lung fibroblast IL-13 | 3.8 |
| ionomycin | | | |
| B lymphocytes PWM | 54.7 | Lung fibroblast IFN | 10.4 |
| | | gamma | |
| B lymphocytes CD40L | 43.2 | Dermal fibroblast | 12.6 |
| and IL-4 | | CCD1070 rest | |
| EOL-1 dbcAMP | 10.7 | Dermal fibroblast | 30.1 |
| | | CCD1070 TNF alpha | |
| EOL-1 dbcAMP | 10.8 | Dermal fibroblast | 2.1 |
| PMA/ionomycin | | CCD1070 IL-1 beta | |
| Dendritic cells none | 6.9 | Dermal fibroblast IFN | 4.5 |
| | | gamma | |
| Dendritic cells LPS | 5.6 | Dermal fibroblast IL-4 | 15.8 |
| Dendritic cells anti- | 5.5 | IBD Colitis 2 | 0.9 |
| CD40 | | | |
| Monocytes rest | 9.9 | IBD Crohn's | 1.2 |
| Monocytes LPS | 5.0 | Colon | 3.2 |
| Macrophages rest | 10.4 | Lung | 2.6 |
| Macrophages LPS | 2.0 | Thymus | 8.6 |
| HUVEC none | 8.1 | Kidney | 100.0 |
| HUVEC starved | 23.7 | | |

TABLE BME
Panel CNS_1

| | Rel. Exp. (%) Ag3050, | | Rel. Exp. (%) Ag3050, |
|---|---|---|---|
| Tissue Name | Run 171694540 | Tissue Name | Run 171694540 |
| BA4 Control | 20.7 | BA17 PSP | 30.4 |
| BA4 Control2 | 35.8 | BA17 PSP2 | 15.7 |
| BA4 | 0.0 | Sub Nigra Control | 38.2 |
| Alzheimer's2 | | | |
| BA4 Parkinson's | 45.7 | Sub Nigra Control2 | 14.3 |
| BA4 | 59.9 | Sub Nigra | 10.4 |
| Parkinson's2 | | Alzheimer's2 | |
| BA4 | 24.7 | Sub Nigra | 46.7 |
| Huntington's | | Parkinson's2 | |
| BA4 | 4.8 | Sub Nigra | 49.0 |
| Huntington's2 | | Huntington's | |
| BA4 PSP | 31.4 | Sub Nigra | 11.6 |
| | | Huntington's2 | |
| BA4 PSP2 | 32.3 | Sub Nigra PSP2 | 6.7 |
| BA4 Depression | 1.3 | Sub Nigra | 9.2 |
| | | Depression | |
| BA4 | 17.3 | Sub Nigra | 15.2 |
| Depression2 | | Depression2 | |
| BA7 Control | 38.2 | Glob Palladus | 23.2 |
| | | Control | |
| BA7 Control2 | 1.2 | Glob Palladus | 7.5 |
| | | Control2 | |
| BA7 | 5.4 | Glob Palladus | 6.1 |
| Alzheimer's2 | | Alzheimer's | |
| BA7 Parkinson's | 43.5 | Glob Palladus | 0.0 |
| | | Alzheimer's2 | |
| BA7 | 57.8 | Glob Palladus | 100.0 |
| Parkinson's2 | | Parkinson's | |
| BA7 | 43.8 | Glob Palladus | 20.2 |
| Huntington's | | Parkinson's2 | |
| BA7 | 59.5 | Glob Palladus PSP | 0.0 |
| Huntington's2 | | | |
| BA7 PSP | 42.6 | Glob Palladus PSP2 | 3.0 |
| BA7 PSP2 | 31.9 | Glob Palladus | 16.4 |
| | | Depression | |
| BA7 Depression | 12.6 | Temp Pole Control | 18.9 |
| BA9 Control | 26.2 | Temp Pole Control2 | 45.7 |
| BA9 Control2 | 19.8 | Temp Pole | 2.6 |
| | | Alzheimer's | |
| BA9 Alzheimer's | 6.6 | Temp Pole | 10.0 |
| | | Alzheimer's2 | |
| BA9 | 13.5 | Temp Pole | 22.8 |
| Alzheimer's2 | | Parkinson's | |
| BA9 Parkinson's | 32.1 | Temp Pole | 16.7 |
| | | Parkinson's2 | |
| BA9 | 30.1 | Temp Pole | 34.9 |
| Parkinson's2 | | Huntington's | |
| BA9 | 38.2 | Temp Pole PSP | 5.8 |
| Huntington's | | | |
| BA9 | 31.6 | Temp Pole PSP2 | 10.7 |
| Huntington's2 | | | |
| BA9 PSP | 17.6 | Temp Pole | 24.3 |
| | | Depression2 | |
| BA9 PSP2 | 2.8 | Cing Gyr Control | 62.9 |
| BA9 Depression | 20.3 | Cing Gyr Control2 | 13.4 |
| BA9 | 11.9 | Cing Gyr | 23.7 |
| Depression2 | | Alzheimer's | |
| BA17 Control | 65.5 | Cing Gyr | 11.7 |
| | | Alzheimer's2 | |
| BA17 Control2 | 31.4 | Cing Gyr Parkinson's | 36.6 |
| BA17 | 11.0 | Cing Gyr | 67.4 |
| Alzheimer's2 | | Parkinson's2 | |
| BA17 | 62.9 | Cing Gyr | 89.5 |
| Parkinson's | | Huntington's | |
| BA17 | 59.9 | Cing Gyr | 26.4 |
| Parkinson's2 | | Huntington's2 | |
| BA17 | 72.2 | Cing Gyr PSP | 11.6 |
| Huntington's | | | |
| BA17 | 38.7 | Cing Gyr PSP2 | 2.0 |
| Huntington's2 | | | |
| BA17 | 12.2 | Cing Gyr Depression | 13.0 |
| Depression | | | |
| BA17 | 42.9 | Cing Gyr | 20.0 |
| Depression2 | | Depression2 | |

CNS_neurodegeneration_v1.0 Summary: Ag3050 This panel does not show differential expression of the NOV72 gene in Alzheimer's disease. However, this expression profile confirms the presence of this gene in the brain. Please see Panel 1.3D for discussion of utility of this gene in the central nervous system.

Panel 1.3D Summary: Ag3050 The NOV72 gene exhibits brain-preferential expression and is a member of a family of proteins that mediates ubiquitin-mediated protein degradation. Misprocessing of proteins involved in ubiquitin-mediated protein degradation is thought to be the cause of many neurodegenerative disorders such as Parkinson's disease, as well as those resulting from CAG repeat expansion genes, such as Huntingtin's disease. Therefore, therapeutic modulation of the expression or function of this gene may affect the protein degradation dysfunction seen in these diseases.

In addition, this gene is expressed at a slightly higher level in cancer cell lines compared to the normal lung, ovary, breast, and colon samples on this panel. This suggests that expression of this gene could be used as a diagnostic marker of cancer. Furthermore, inhibition of this gene product using small molecule drugs may be useful in the treatment of cancer in these tissues.

Among tissues with metabolic function, this gene is has a low level of expression in pancreas, thyroid, pituitary, heart, skeletal muscle, and adipose. This gene product may be a small molecule target for the treatment of metabolic and endocrine diseases, including obesity and Types 1 and 2 diabetes.

Panel 4D Summary: Ag3050 The NOV72 gene is expressed at moderate to low levels (CT=29-34) in a wide range of cell types and tissues of significance in the immune response in health and disease, Highest expression of this gene is seen in kidney tissue (CT=29.36). Therefore, targeting of this gene product with a small molecule drug or antibody therapeutic may modulate the functions of cells of the immune system as well as resident tissue cells and lead to improvement of the symptoms of patients suffering from autoimmune and inflammatory diseases such as asthma, allergies, inflammatory bowel disease, lupus erythematosus, and arthritis, including osteoarthritis and rheumatoid arthritis.

Panel CNS_{—}1 Summary: Ag3050 This panel confirms expression of the NOV72 gene in the brain. Please see Panel 1.3D for discussion of utility of this gene in the central nervous system.

NOV73

Expression of gene NOV73 was assessed using the primer-probe set Ag3030, described in Table BNA.

TABLE BNA
Probe Name Ag3030

| | | | Start | |
|---|---|---|---|---|
| Primers | Seqences | Length | Position | SEQ ID NO: |
| Forward | 5′-tgaaattcagaaccaggaaatg-3′ | 22 | 838 | 1285 |
| Probe | TET-5′-aaagagtgcttagcaggcacctccct-3′-TAMRA | 26 | 872 | 1286 |
| Reverse | 5′-aaccctggcaatatgattcata-3′ | 22 | 914 | 1287 |

CNS_neurodegeneration_v1.0 Summary: Ag3030 Expression of this gene is low/undetectable (CTs>34.5) across all of the samples on this panel (data not shown).

Panel 1.3D Summary: Ag3030 Expression of this gene is low/undetectable (CTs>34.5) across all of the samples on this panel (data not shown).

Panel 2D Summary: Ag3030 Expression of this gene is low/undetectable (CTs>35) across all of the samples on this panel (data not shown).

Panel 3D Summary: Ag3030 Expression of this gene is low/undetectable (CTs>35) across all of the samples on this panel (data not shown).

Panel 4D Summary: Ag3030 Expression of this gene is low/undetectable (CTs>35) across all of the samples on this panel (data not shown).

Panel 5 Islet Summary: Ag3030 Expression of this gene is low/undetectable (CTs>35) across all of the samples on this panel (data not shown).

NOV74

Expression of gene NOV74 was assessed using the primer-probe set Ag3016, described in Table BOA. Results of the RTQ-PCR runs are shown in Tables BOB, BOC, BOD and BOE.

TABLE BOA
Probe Name Ag3016

| | | | Start | |
|---|---|---|---|---|
| Primers | Seqences | Length | Position | SEQ ID NO: |
| Forward | 5′-atctcagtgacctgctctcaga-3′ | 22 | 83 | 1288 |
| Probe | TET-5′-cagtggctgctacagcctcccaag-3′-TAMRA | 24 | 108 | 1289 |
| Reverse | 5′-aaatcttcagggtgacctcatt-3′ | 22 | 142 | 1290 |

TABLE BOB
CNS_neurodegeneration_v1.0

| | Rel. Exp. (%) Ag3016, | | Rel. Exp. (%) Ag3016, |
|---|---|---|---|
| Tissue Name | Run 209820675 | Tissue Name | Run 209820675 |
| AD 1 Hippo | 4.6 | Control (Path) 3 | 3.1 |
| | | Temporal Ctx | |
| AD 2 Hippo | 7.9 | Control (Path) 4 | 33.9 |
| | | Temporal Ctx | |
| AD 3 Hippo | 2.9 | AD 1 Occipital Ctx | 17.9 |
| AD 4 Hippo | 3.3 | AD 2 Occipital Ctx | 0.0 |
| | | (Missing) | |
| AD 5 Hippo | 100.0 | AD 3 Occipital Ctx | 7.5 |
| AD 6 Hippo | 8.5 | AD 4 Occipital Ctx | 12.5 |
| Control 2 Hippo | 5.4 | AD 5 Occipital Ctx | 11.7 |
| Control 4 Hippo | 1.8 | AD 6 Occipital Ctx | 12.9 |
| Control (Path) 3 | 1.5 | Control 1 Occipital | 3.7 |
| Hippo | | Ctx | |
| AD 1 Temporal Ctx | 10.8 | Control 2 Occipital | 18.8 |
| | | Ctx | |
| AD 2 Temporal Ctx | 20.4 | Control 3 Occipital | 0.7 |
| | | Ctx | |
| AD 3 Temporal Ctx | 3.5 | Control 4 Occipital | 5.8 |
| | | Ctx | |
| AD 4 Temporal Ctx | 22.2 | Control (Path) 1 | 50.3 |
| | | Occipital Ctx | |
| AD 5 Inf Temporal | 60.7 | Control (Path) 2 | 23.3 |
| Ctx | | Occipital Ctx | |
| AD 5 Sup Temporal | 17.7 | Control (Path) 3 | 1.5 |
| Ctx | | Occipital Ctx | |
| AD 6 Inf Temporal | 23.7 | Control (Path) 4 | 36.6 |
| Ctx | | Occipital Ctx | |
| AD 6 Sup Temporal | 23.7 | Control 1 Parietal | 6.1 |
| Ctx | | Ctx | |
| Control 1 Temporal | 5.6 | Control 2 Parietal | 28.7 |
| Ctx | | Ctx | |
| Control 2 Temporal | 5.9 | Control 3 Parietal | 21.3 |
| Ctx | | Ctx | |
| Control 3 Temporal | 11.1 | Control (Path) 1 | 49.3 |
| Ctx | | Parietal Ctx | |
| Control 3 Temporal | 6.3 | Control (Path) 2 | 27.0 |
| Ctx | | Parietal Ctx | |
| Control (Path) 1 | 45.4 | Control (Path) 3 | 5.6 |
| Temporal Ctx | | Parietal Ctx | |
| Control (Path) 2 | 36.9 | Control (Path) 4 | 39.0 |
| Temporal Ctx | | Parietal Ctx | |

TABLE BOC
Panel 1.3D

| | Rel. Exp. (%) Ag3016, | | Rel. Exp. (%) Ag3016, |
|---|---|---|---|
| Tissue Name | Run 167819111 | Tissue Name | Run 167819111 |
| Liver adenocarcinoma | 0.0 | Kidney (fetal) | 9.2 |
| Pancreas | 0.0 | Renal ca. 786-0 | 0.0 |
| Pancreatic ca. CAPAN 2 | 0.0 | Renal ca. A498 | 0.0 |
| Adrenal gland | 0.0 | Renal ca. RXF 393 | 0.0 |
| Thyroid | 0.0 | Renal ca. ACHN | 0.0 |
| Salivary gland | 0.0 | Renal ca. UO-31 | 0.0 |
| Pituitary gland | 0.0 | Renal ca. TK-10 | 0.0 |
| Brain (fetal) | 99.3 | Liver | 0.0 |
| Brain (whole) | 96.6 | Liver (fetal) | 0.0 |
| Brain (amygdala) | 21.5 | Liver ca. | 0.0 |
| | | (hepatoblast) HepG2 | |
| Brain (cerebellum) | 12.0 | Lung | 0.0 |
| Brain (hippocampus) | 24.7 | Lung (fetal) | 6.2 |
| Brain (substantia nigra) | 15.0 | Lung ca. (small cell) | 0.0 |
| | | LX-1 | |
| Brain (thalamus) | 0.0 | Lung ca. (small cell) | 8.2 |
| | | NCI-H69 | |
| Cerebral Cortex | 100.0 | Lung ca. (s.cell var.) | 6.9 |
| | | SHP-77 | |
| Spinal cord | 0.0 | Lung ca. (large | 0.0 |
| | | cell)NCI-H460 | |
| glio/astro U87-MG | 46.0 | Lung ca. (non-sm. | 10.9 |
| | | cell) A549 | |
| glio/astro U-118-MG | 0.0 | Lung ca. (non-s.cell) | 6.0 |
| | | NCI-H23 | |
| astrocytoma SW1783 | 6.3 | Lung ca. (non-s.cell) | 0.0 |
| | | HOP-62 | |
| neuro*; met SK-N-AS | 0.0 | Lung ca. (non-s.cl) | 0.0 |
| | | NCI-H522 | |
| astrocytoma SF-539 | 7.1 | Lung ca. (squam.) | 0.0 |
| | | SW 900 | |
| astrocytoma SNB-75 | 0.0 | Lung ca. (squam.) | 0.0 |
| | | NCI-H596 | |
| glioma SNB-19 | 0.0 | Mammary gland | 0.0 |
| glioma U251 | 0.0 | Breast ca.* (pl.ef) | 0.0 |
| | | MCF-7 | |
| glioma SF-295 | 32.5 | Breast ca.* (pl.ef) | 0.0 |
| | | MDA-MB-231 | |
| Heart (fetal) | 0.0 | Breast ca.* (pl.ef) | 81.2 |
| | | T47D | |
| Heart | 0.0 | Breast ca. BT-549 | 0.0 |
| Skeletal muscle (fetal) | 10.6 | Breast ca. MDA-N | 0.0 |
| Skeletal muscle | 0.0 | Ovary | 0.0 |
| Bone marrow | 0.0 | Ovarian ca. OVCAR-3 | 0.0 |
| Thymus | 7.0 | Ovarian ca. OVCAR-4 | 4.5 |
| Spleen | 0.0 | Ovarian ca. OVCAR-5 | 0.0 |
| Lymph node | 6.3 | Ovarian ca. OVCAR-8 | 100.0 |
| Colorectal | 0.0 | Ovarian ca. IGROV-1 | 0.0 |
| Stomach | 0.0 | Ovarian ca.* (ascites) | 72.7 |
| | | SK-OV-3 | |
| Small intestine | 0.0 | Uterus | 0.0 |
| Colon ca. SW480 | 0.0 | Placenta | 0.0 |
| Colon ca.* | 0.0 | Prostate | 0.0 |
| SW620(SW480 met) | | | |
| Colon ca. HT29 | 0.0 | Prostate ca.* (bone | 0.0 |
| | | met)PC-3 | |
| Colon ca. HCT-116 | 0.0 | Testis | 15.4 |
| Colon ca. CaCo-2 | 0.0 | Melanoma | 19.5 |
| | | Hs688(A).T | |
| Colon ca. | 0.0 | Melanoma* (met) | 0.0 |
| tissue(ODO3866) | | Hs688(B).T | |
| Colon ca. HCC-2998 | 0.0 | Melanoma UACC-62 | 57.0 |
| Gastric ca.* (liver met) | 0.0 | Melanoma M14 | 26.8 |
| NCI-N87 | | | |
| Bladder | 0.0 | Melanoma LOX | 0.0 |
| | | IMVI | |
| Trachea | 0.0 | Melanoma* (met) | 19.8 |
| | | SK-MEL-5 | |
| Kidney | 0.0 | Adipose | 0.0 |

TABLE BOD
Panel 4D

| | Rel. Exp. (%) | | Rel. Exp. (%) |
|---|---|---|---|
| | Ag3016, Run | | Ag3016, Run |
| Tissue Name | 164404251 | Tissue Name | 164404251 |
| Secondary Th1 act | 0.0 | HUVEC IL-1 beta | 0.0 |
| Secondary Th2 act | 6.2 | HUVEC IFN gamma | 0.0 |
| Secondary Tr1 act | 0.0 | HUVEC TNF alpha + IFN | 0.0 |
| | | gamma | |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC | 0.0 |
| | | none | |
| Primary Th1 act | 0.0 | Lung Microvascular EC | 0.0 |
| | | TNF alpha + IL-1 beta | |
| Primary Th2 act | 0.0 | Microvascular Dermal EC | 0.0 |
| | | none | |
| Primary Tr1 act | 0.0 | Microsvasular Dermal EC | 0.0 |
| | | TNF alpha + IL-1 beta | |
| Primary Th1 rest | 0.0 | Bronchial epithelium | 0.0 |
| | | TNF alpha + IL1 beta | |
| Primary Th2 rest | 0.0 | Small airway epithelium | 0.0 |
| | | none | |
| Primary Tr1 rest | 0.0 | Small airway epithelium | 0.0 |
| | | TNF alpha + IL-1 beta | |
| CD45RA CD4 | 0.0 | Coronery artery SMC rest | 0.0 |
| lymphocyte act | | | |
| CD45RO CD4 | 0.0 | Coronery artery SMC | 0.0 |
| lymphocyte act | | TNF alpha + IL-1 beta | |
| CD8 lymphocyte act | 0.0 | Astrocytes rest | 13.7 |
| Secondary CD8 | 0.0 | Astrocytes TNF alpha + | 0.0 |
| lymphocyte rest | | IL-1 beta | |
| Secondary CD8 | 0.0 | KU-812 (Basophil) rest | 0.0 |
| lymphocyte act | | | |
| CD4 lymphocyte none | 0.0 | KU-812 (Basophil) | 0.0 |
| | | PMA/ionomycin | |
| 2ry Th1/Th2/Tr1_anti- | 0.0 | CCD1106 (Keratinocytes) | 0.0 |
| CD95 CH11 | | none | |
| LAK cells rest | 0.0 | CCD1106 (Keratinocytes) | 0.0 |
| | | TNF alpha + IL-1 beta | |
| LAK cells IL-2 | 0.0 | Liver cirrhosis | 56.6 |
| LAK cells IL-2 + IL-12 | 0.0 | Lupus kidney | 0.0 |
| LAK cells IL-2 + IFN | 0.0 | NCI-H292 none | 0.0 |
| gamma | | | |
| LAK cells IL-2 + IL-18 | 0.0 | NCI-H292 IL-4 | 0.0 |
| LAK cells | 0.0 | NCI-H292 IL-9 | 0.0 |
| PMA/ionomycin | | | |
| NK Cells IL-2 rest | 0.0 | NCI-H292 IL-13 | 0.0 |
| Two Way MLR 3 day | 0.0 | NCI-H292 IFN gamma | 0.0 |
| Two Way MLR 5 day | 0.0 | HPAEC none | 0.0 |
| Two Way MLR 7 day | 0.0 | HPAEC TNF alpha + IL-1 | 0.0 |
| | | beta | |
| PBMC rest | 0.0 | Lung fibroblast none | 0.0 |
| PBMC PWM | 0.0 | Lung fibroblast TNF alpha + | 0.0 |
| | | IL-1 beta | |
| PBMC PHA-L | 0.0 | Lung fibroblast IL-4 | 0.0 |
| Ramos (B cell) none | 0.0 | Lung fibroblast IL-9 | 0.0 |
| Ramos (B cell) | 0.0 | Lung fibroblast IL-13 | 0.0 |
| ionomycin | | | |
| B lymphocytes PWM | 0.0 | Lung fibroblast IFN | 0.0 |
| | | gamma | |
| B lymphocytes CD40L | 0.0 | Dermal fibroblast | 0.0 |
| and IL-4 | | CCD1070 rest | |
| EOL-1 dbcAMP | 0.0 | Dermal fibroblast | 0.0 |
| | | CCD1070 TNF alpha | |
| EOL-1 dbcAMP | 0.0 | Dermal fibroblast | 0.0 |
| PMA/ionomycin | | CCD1070 IL-1 beta | |
| Dendritic cells none | 0.0 | Dermal fibroblast IFN | 7.6 |
| | | gamma | |
| Dendritic cells LPS | 0.0 | Dermal fibroblast IL-4 | 6.0 |
| Dendritic cells anti- | 0.0 | IBD Colitis 2 | 5.6 |
| CD40 | | | |
| Monocytes rest | 0.0 | IBD Crohn's | 0.0 |
| Monocytes LPS | 0.0 | Colon | 0.0 |
| Macrophages rest | 0.0 | Lung | 6.7 |
| Macrophages LPS | 0.0 | Thymus | 4.3 |
| HUVEC none | 0.0 | Kidney | 100.0 |
| HUVEC starved | 0.0 | | |

TABLE BOE
Panel CNS_1

| | Rel. Exp. (%) Ag3016, | | Rel. Exp. (%) Ag3016, |
|---|---|---|---|
| Tissue Name | Run 171688428 | Tissue Name | Run 171688428 |
| BA4 Control | 9.9 | BA17 PSP | 28.5 |
| BA4 Control2 | 12.0 | BA17 PSP2 | 17.8 |
| BA4 | 2.9 | Sub Nigra Control | 5.4 |
| Alzheimer's2 | | | |
| BA4 Parkinson's | 28.9 | Sub Nigra Control2 | 0.0 |
| BA4 | 35.1 | Sub Nigra | 7.7 |
| Parkinson's2 | | Alzheimer's2 | |
| BA4 | 12.2 | Sub Nigra | 15.1 |
| Huntington's | | Parkinson's2 | |
| BA4 | 8.3 | Sub Nigra | 3.5 |
| Huntington's2 | | Huntington's | |
| BA4 PSP | 11.4 | Sub Nigra | 0.0 |
| | | Huntington's2 | |
| BA4 PSP2 | 17.9 | Sub Nigra PSP2 | 0.0 |
| BA4 Depression | 9.7 | Sub Nigra | 5.2 |
| | | Depression | |
| BA4 | 11.7 | Sub Nigra | 14.8 |
| Depression2 | | Depression2 | |
| BA7 Control | 31.4 | Glob Palladus | 5.0 |
| | | Control | |
| BA7 Control2 | 11.7 | Glob Palladus | 0.0 |
| | | Control2 | |
| BA7 | 20.2 | Glob Palladus | 0.0 |
| Alzheimer's2 | | Alzheimer's | |
| BA7 Parkinson's | 15.3 | Glob Palladus | 10.6 |
| | | Alzheimer's2 | |
| BA7 | 59.0 | Glob Palladus | 58.2 |
| Parkinson's2 | | Parkinson's | |
| BA7 | 43.2 | Glob Palladus | 0.0 |
| Huntington's | | Parkinson's2 | |
| BA7 | 46.0 | Glob Palladus PSP | 0.0 |
| Huntington's2 | | | |
| BA7 PSP | 37.6 | Glob Palladus PSP2 | 5.0 |
| BA7 PSP2 | 21.9 | Glob Palladus | 5.7 |
| | | Depression | |
| BA7 Depression | 21.9 | Temp Pole Control | 5.9 |
| BA9 Control | 14.8 | Temp Pole Control2 | 0.0 |
| BA9 Control2 | 24.5 | Temp Pole | 0.0 |
| | | Alzheimer's | |
| BA9 Alzheimer's | 10.2 | Temp Pole | 9.4 |
| | | Alzheimer's2 | |
| BA9 | 12.2 | Temp Pole | 30.4 |
| Alzheimer's2 | | Parkinson's | |
| BA9 Parkinson's | 52.1 | Temp Pole | 37.1 |
| | | Parkinson's2 | |
| BA9 | 45.4 | Temp Pole | 11.5 |
| Parkinson's2 | | Huntington's | |
| BA9 | 20.2 | Temp Pole PSP | 19.3 |
| Huntington's | | | |
| BA9 | 35.1 | Temp Pole PSP2 | 0.0 |
| Huntington's2 | | | |
| BA9 PSP | 24.3 | Temp Pole | 12.5 |
| | | Depression2 | |
| BA9 PSP2 | 9.5 | Cing Gyr Control | 21.0 |
| BA9 Depression | 9.8 | Cing Gyr Control2 | 16.2 |
| BA9 | 12.2 | Cing Gyr | 0.0 |
| Depression2 | | Alzheimer's | |
| BA17 Control | 100.0 | Cing Gyr | 0.0 |
| | | Alzheimer's2 | |
| BA17 Control2 | 40.9 | Cing Gyr Parkinson's | 10.5 |
| BA17 | 28.9 | Cing Gyr | 0.0 |
| Alzheimer's2 | | Parkinson's2 | |
| BA17 | 94.6 | Cing Gyr | 35.4 |
| Parkinson's | | Huntington's | |
| BA17 | 28.1 | Cing Gyr | 5.0 |
| Parkinson's2 | | Huntington's2 | |
| BA17 | 25.9 | Cing Gyr PSP | 0.0 |
| Huntington's | | | |
| BA17 | 19.3 | Cing Gyr PSP2 | 9.8 |
| Huntington's2 | | | |
| BA17 | 33.4 | Cing Gyr Depression | 5.0 |
| Depression | | | |
| BA17 | 56.3 | Cing Gyr | 24.8 |
| Depression2 | | Depression2 | |

CNS_neurodegeneration_v1.0 Summary: Ag3016 This panel does not show differential expression of the NOV74 gene in Alzheimer's disease. However, this expression profile confirms the presence of this gene in the brain. Please see Panel 1.3D for discussion of utility of this gene in the central nervous system.

Panel 1.3D Summary: Ag3016 The NOV74 gene represents a dual specificity phosphatase that is expressed preferentially at low to moderate levels across the CNS. Dual-specificity phosphatases comprise a family of MAP kinase regulating enzymes, members of which are upregulated in brains subjected to insults such as ischemia and seizure activity. MAP kinases are kown to regulate neurotrophic and neurotoxic pathways. Consequently, agents that modulate the activity of this gene may have utility in attenuating the apoptotic and neurodegenerative processes following brain insults.

In addition, there are low but significant levels of expression in samples derived from breast cancer, ovarian cancer, and melanoma cell lines. Thus, expression of this gene could be used to differentiate between these samples and other samples on this panel and as a marker to detect the presence of these cancers. Furthermore, therapeutic modulation of the expression or function of this gene may be effective in the treatment of breast cancer, ovarian cancer, and melanoma.

References:

Wiessner C. The dual specificity phosphatase PAC-1 is transcriptionally induced in the rat brain following transient forebrain ischemia. Brain Res Mol Brain Res February 1995;28(2):353-6

PAC-1 mRNA has previously been found only in activated T-cells in vitro and in vivo. The gene encodes a dual specificity protein phosphatase that regulates MAP kinase activity. Here, I describe that PAC-1 mRNA is induced also in neurons in the rat brain following 30 min of forebrain ischemia. At 6, 12 and 24 h after ischemia, PAC-1 mRNA was found most prominently in hippocampal cells which are resistant to 30 min of forebrain ischemia, but not in the selectively vulnerable CA1 sector. At later time points and in control animals no PAC-1 mRNA could be detected in any brain region. The protein-tyrosine/threonine phosphatase PAC-1, therefore, may be involved in adaptational responses of hippocampal cells resistant to ischemic injury.

Boschert U, Muda M, Camps M, Dickinson R, Arkinstall S. Induction of the dual specificity phosphatase PAC1 in rat brain following seizure activity. Neuroreport Sep. 29, 1997;8(14):3077-80

Recurrent seizure activity leads to delayed neuronal death as well as to inflammatory responses involving microglia in hippocampal subfields CA1, CA3 and CA4. Since mitogen activated protein (MAP) kinases control neuronal aptosis and trigger regeneration of inflammatory cytokines, their activation state could determine seizure-related brain damage. PAC1 is a dual specificity protein phosphotase inactivating MAP kinases which we have found to be undetectable in normal brain. Despite this, kainic acid-induced seizure activity leads to rapid (approximately 3 h) but transient appearance of PAC1 mRNA in granule cells of the denate gyrus as well as in pyramidal CA1 neurons. This pattern changed with time and after 2-3 days PAC1 was induced in dying CA1 and CA3 neurons. At this time PAC1 mRNA was also expressed in white matter microglia as well as in microglia invading the damaged hippocampus. PAC1 may play an important role controlling MAP kinase involvement in both neuronal death and neuro-inflammation following excitotoxic damage.

Panel 4D Summary: Ag3016 The NOV74 gene is only expressed at detectable levels in the kidney (CT=34.2) among the samples on this panel. Thus, expression of this gene could be used to distinguish kidney from the other samples on this panel. In addition, the dual-specificity protein phospatase encoded for by this gene could allow cells within the kidney to respond to specific microenvironmental signals. Furthermore, small molecule therapies designed with the protein encoded for by this gene could,modulate kidney function and be important in the treatment of inflammatory or autoimmune diseases that affect the kidney, including lupus and glomerulonephritis.

Panel CNS_{—}1 Summary: Ag3016 This panel confirms expression of the NOV74 gene in the brain. Please see Panel 1.3D for discussion of utility of this gene in the central nervous system.

NOV75

Expression of gene NOV75 was assessed using the primer-probe sets Ag3020 and Ag2968, described in Tables BPA and BPB. Results of the RTQ-PCR runs are shown in Tables BPC, BPD, BPE, BPF and BPG.

TABLE BPA
Probe Name Ag3020

| | | | Start | |
|---|---|---|---|---|
| Primers | Seqences | Length | Position | SEQ ID NO: |
| Forward | 5′-ggaatcacccacattctgaat-3′ | 21 | 265 | 1291 |
| Probe | TET-5′-cgtttacactggccccgaattctaca-3′-TAMRA | 26 | 303 | 1292 |
| Reverse | 5′-cctctacacccaggtactggat-3′ | 22 | 340 | 1293 |

TABLE BPB
Probe Name Ag2968

| | | | Start | |
|---|---|---|---|---|
| Primers | Seqences | Length | Position | SEQ ID NO: |
| Forward | 5′-ggaatcacccacattctgaat-3′ | 21 | 265 | 1294 |
| Probe | TET-5′-cgtttacactggccccgaattctaca-3′-TAMRA | 26 | 303 | 1295 |
| Reverse | 5′-cctctacacccaggtactggat-3′ | 22 | 340 | 1296 |

TABLE BPC
General_screening_panel_v1.4

| | Rel. Exp. (%) Ag3020, | | Rel. Exp. (%) Ag3020, |
|---|---|---|---|
| Tissue Name | Run 221998694 | Tissue Name | Run 221998694 |
| Adipose | 0.4 | Renal ca. TK-10 | 0.0 |
| Melanoma* | 0.0 | Bladder | 0.1 |
| Hs688(A).T | | | |
| Melanoma* | 0.0 | Gastric ca. (liver met.) | 0.0 |
| Hs688(B).T | | NCI-N87 | |
| Melanoma* M14 | 0.0 | Gastric ca. KATO III | 0.0 |
| Melanoma* | 0.0 | Colon ca. SW-948 | 0.0 |
| LOXIMVI | | | |
| Melanoma* SK- | 0.0 | Colon ca. SW480 | 0.2 |
| MEL-5 | | | |
| Squamous cell | 0.0 | Colon ca.* (SW480 | 0.0 |
| carcinoma SCC-4 | | met) SW620 | |
| Testis Pool | 0.0 | Colon ca. HT29 | 0.0 |
| Prostate ca.* (bone | 0.0 | Colon ca. HCT-116 | 0.0 |
| met) PC-3 | | | |
| Prostate Pool | 0.2 | Colon ca. CaCo-2 | 0.0 |
| Placenta | 0.1 | Colon cancer tissue | 31.0 |
| Uterus Pool | 0.0 | Colon ca. SW1116 | 0.0 |
| Ovarian ca. | 0.0 | Colon ca. Colo-205 | 100.0 |
| OVCAR-3 | | | |
| Ovarian ca. SK-OV-3 | 0.3 | Colon ca. SW-48 | 5.7 |
| Ovarian ca. | 0.0 | Colon Pool | 0.0 |
| OVCAR-4 | | | |
| Ovarian ca. | 0.1 | Small Intestine Pool | 0.0 |
| OVCAR-5 | | | |
| Ovarian ca. IGROV-1 | 18.9 | Stomach Pool | 0.0 |
| Ovarian ca. | 0.0 | Bone Marrow Pool | 0.0 |
| OVCAR-8 | | | |
| Ovary | 0.1 | Fetal Heart | 12.7 |
| Breast ca. MCF-7 | 0.0 | Heart Pool | 4.9 |
| Breast ca. MDA- | 0.0 | Lymph Node Pool | 0.1 |
| MB-231 | | | |
| Breast ca. BT 549 | 0.0 | Fetal Skeletal Muscle | 23.3 |
| Breast ca. T47D | 0.0 | Skeletal Muscle Pool | 0.0 |
| Breast ca. MDA-N | 0.0 | Spleen Pool | 0.0 |
| Breast Pool | 0.0 | Thymus Pool | 0.0 |
| Trachea | 0.3 | CNS cancer (glio/astro) | 0.0 |
| | | U87-MG | |
| Lung | 0.0 | CNS cancer (glio/astro) | 0.1 |
| | | U-118-MG | |
| Fetal Lung | 0.2 | CNS cancer | 0.0 |
| | | (neuro; met) SK-N-AS | |
| Lung ca. NCI-N417 | 0.0 | CNS cancer (astro) SF- | 0.7 |
| | | 539 | |
| Lung ca. LX-1 | 12.7 | CNS cancer (astro) | 0.1 |
| | | SNB-75 | |
| Lung ca. NCI-H146 | 0.1 | CNS cancer (glio) | 15.9 |
| | | SNB-19 | |
| Lung ca. SHP-77 | 0.0 | CNS cancer (glio) SF- | 0.5 |
| | | 295 | |
| Lung ca. A549 | 0.0 | Brain (Amygdala) Pool | 0.1 |
| Lung ca. NCI-H526 | 0.0 | Brain (cerebellum) | 0.0 |
| Lung ca. NCI-H23 | 0.0 | Brain (fetal) | 0.1 |
| Lung ca. NCI-H460 | 0.0 | Brain (Hippocampus) | 0.1 |
| | | Pool | |
| Lung ca. HOP-62 | 0.1 | Cerebral Cortex Pool | 0.1 |
| Lung ca. NCI-H522 | 0.0 | Brain (Substantia nigra) | 0.1 |
| | | Pool | |
| Liver | 0.0 | Brain (Thalamus) Pool | 0.1 |
| Fetal Liver | 0.1 | Brain (whole) | 0.1 |
| Liver ca. HepG2 | 0.0 | Spinal Cord Pool | 0.0 |
| Kidney Pool | 0.1 | Adrenal Gland | 0.0 |
| Fetal Kidney | 0.1 | Pituitary gland Pool | 0.0 |
| Renal ca. 786-0 | 0.0 | Salivary Gland | 0.1 |
| Renal ca. A498 | 0.0 | Thyroid (female) | 0.0 |
| Renal ca. ACHN | 0.0 | Pancreatic ca. CAPAN2 | 0.4 |
| Renal ca. UO-31 | 0.0 | Pancreas Pool | 0.0 |

TABLE BPD
Panel 1.3D

| | Rel. Exp. (%) | Rel. Exp. (%) | | Rel. Exp. (%) | Rel. Exp. (%) |
|---|---|---|---|---|---|
| | Ag2968, Run | Ag3020, Run | | Ag2968, Run | Ag3020, Run |
| Tissue Name | 166220058 | 167819114 | Tissue Name | 166220058 | 167819114 |
| Liver | 0.0 | 0.1 | Kidney (fetal) | 0.0 | 0.3 |
| adenocarcinoma | | | | | |
| Pancreas | 0.0 | 0.0 | Renal ca. 786-0 | 0.0 | 0.0 |
| Pancreatic ca. | 0.1 | 0.2 | Renal ca. | 0.0 | 0.0 |
| CAPAN 2 | | | A498 | | |
| Adrenal gland | 0.0 | 0.0 | Renal ca. RXF | 0.0 | 0.0 |
| | | | 393 | | |
| Thyroid | 0.3 | 1.6 | Renal ca. | 0.0 | 0.0 |
| | | | ACHN | | |
| Salivary gland | 0.8 | 0.6 | Renal ca. UO- | 0.0 | 0.0 |
| | | | 31 | | |
| Pituitary gland | 0.0 | 0.0 | Renal ca. TK- | 0.0 | 0.0 |
| | | | 10 | | |
| Brain (fetal) | 0.0 | 0.1 | Liver | 0.0 | 0.0 |
| Brain (whole) | 0.4 | 1.0 | Liver (fetal) | 0.5 | 0.0 |
| Brain (amygdala) | 0.2 | 1.0 | Liver ca. | 0.0 | 0.0 |
| | | | (hepatoblast) | | |
| | | | HepG2 | | |
| Brain (cerebellum) | 0.0 | 0.0 | Lung | 0.0 | 0.5 |
| Brain | 0.0 | 0.0 | Lung (fetal) | 0.0 | 0.0 |
| (hippocampus) | | | | | |
| Brain (substantia | 0.2 | 0.1 | Lung ca. | 10.7 | 16.2 |
| nigra) | | | (small cell) | | |
| | | | LX-1 | | |
| Brain (thalamus) | 0.0 | 0.0 | Lung ca. | 0.0 | 0.5 |
| | | | (small cell) | | |
| | | | NCI-H69 | | |
| Cerebral Cortex | 0.4 | 0.1 | Lung ca. | 0.0 | 0.0 |
| | | | (s.cell var.) | | |
| | | | SHP-77 | | |
| Spinal cord | 0.0 | 0.0 | Lung ca. (large | 0.0 | 0.0 |
| | | | cell)NCI-H460 | | |
| glio/astro U87-MG | 0.0 | 0.0 | Lung ca. (non- | 0.0 | 0.2 |
| | | | sm. cell) A549 | | |
| glio/astro U-118- | 0.0 | 0.1 | Lung ca. (non- | 0.0 | 0.0 |
| MG | | | s.cell) NCI- | | |
| | | | H23 | | |
| astrocytoma | 0.0 | 0.0 | Lung ca. (non- | 0.1 | 0.1 |
| SW1783 | | | s.cell) HOP-62 | | |
| neuro*; met SK-N- | 0.0 | 0.0 | Lung ca. (non- | 0.0 | 0.0 |
| AS | | | s.cl) NCI- | | |
| | | | H522 | | |
| astrocytoma SF- | 12.1 | 8.4 | Lung ca. | 0.0 | 0.0 |
| 539 | | | (squam.) SW | | |
| | | | 900 | | |
| astrocytoma SNB- | 0.0 | 0.3 | Lung ca. | 0.1 | 0.4 |
| 75 | | | (squam.) NCI- | | |
| | | | H596 | | |
| glioma SNB-19 | 0.0 | 0.0 | Mammary | 0.2 | 0.2 |
| | | | gland | | |
| glioma U251 | 0.4 | 0.0 | Breast ca.* | 0.0 | 0.0 |
| | | | (pl.ef) MCF-7 | | |
| glioma SF-295 | 0.3 | 0.3 | Breast ca.* | 0.0 | 0.0 |
| | | | (pl.ef) MDA- | | |
| | | | MB-231 | | |
| Heart (fetal) | 7.4 | 26.8 | Breast ca.* | 0.0 | 0.0 |
| | | | (pl.ef) T47D | | |
| Heart | 29.9 | 35.4 | Breast ca. BT- | 0.0 | 0.0 |
| | | | 549 | | |
| Skeletal muscle | 10.8 | 33.9 | Breast ca. | 0.0 | 0.0 |
| (fetal) | | | MDA-N | | |
| Skeletal muscle | 100.0 | 100.0 | Ovary | 0.0 | 0.1 |
| Bone marrow | 0.1 | 0.6 | Ovarian ca. | 0.0 | 0.0 |
| | | | OVCAR-3 | | |
| Thymus | 0.1 | 0.1 | Ovarian ca. | 0.0 | 0.0 |
| | | | OVCAR-4 | | |
| Spleen | 0.0 | 0.0 | Ovarian ca. | 0.0 | 0.6 |
| | | | OVCAR-5 | | |
| Lymph node | 0.0 | 0.0 | Ovarian ca. | 0.0 | 0.0 |
| | | | OVCAR-8 | | |
| Colorectal | 0.2 | 0.2 | Ovarian ca. | 26.2 | 26.2 |
| | | | IGROV-1 | | |
| Stomach | 0.0 | 0.0 | Ovarian ca.* | 0.2 | 1.0 |
| | | | (ascites) SK- | 0.2 | 1.0 |
| | | | OV-3 | | |
| Small intestine | 0.0 | 0.0 | Uterus | 0.0 | 0.0 |
| Colon ca. SW480 | 0.0 | 0.2 | Placenta | 1.0 | 0.0 |
| Colon ca.* | 1.6 | 6.1 | Prostate | 0.2 | 0.1 |
| SW620(SW480 | | | | | |
| met) | | | | | |
| Colon ca. HT29 | 0.0 | 0.0 | Prostate ca.* | 0.0 | 0.0 |
| | | | (bone met) PC-3 | | |
| Colon ca. HCT- | 0.0 | 0.0 | Testis | 0.2 | 0.2 |
| 116 | | | | | |
| Colon ca. CaCo-2 | 0.0 | 0.0 | Melanoma | 0.0 | 0.0 |
| | | | Hs688(A).T | | |
| Colon ca. | 21.9 | 30.6 | Melanoma* | 0.0 | 0.0 |
| tissue(ODO3866) | | | (met) | | |
| | | | Hs688(B).T | | |
| Colon ca. HCC- | 0.0 | 0.5 | Melanoma | 0.0 | 0.0 |
| 2998 | | | UACC-62 | | |
| Gastric ca.* (liver | 0.0 | 0.0 | Melanoma | 0.0 | 0.0 |
| met) NCI-N87 | | | M14 | | |
| Bladder | 0.1 | 0.0 | Melanoma | 0.0 | 0.0 |
| | | | LOX IMVI | | |
| Trachea | 0.8 | 0.6 | Melanoma* | 0.0 | 0.0 |
| | | | (met) SK- | | |
| | | | MEL-5 | | |
| Kidney | 0.0 | 0.0 | Adipose | 1.1 | 1.7 |

TABLE BPE
Panel 3D

| | Rel. Exp. (%) | | Rel. Exp. (%) |
|---|---|---|---|
| | Ag2968, Run | | Ag2968, Run |
| Tissue Name | 170188142 | Tissue Name | 170188142 |
| Daoy-Medulloblastoma | 0.0 | Ca Ski-Cervical epidermoid | 0.0 |
| | | carcinoma (metastasis) | |
| TE671-Medulloblastoma | 2.2 | ES-2-Ovarian clear cell | 0.0 |
| | | carcinoma | |
| D283 Med- | 0.0 | Ramos-Stimulated with | 0.0 |
| Medulloblastoma | | PMA/ionomycin 6 h | |
| PFSK-1-Primitive | 0.0 | Ramos-Stimulated with | 0.0 |
| Neuroectodermal | | PMA/ionomycin 14 h | |
| XF-498-CNS | 0.0 | MEG-01-Chronic | 0.0 |
| | | myelogenous leukemia | |
| | | (megokaryoblast) | |
| SNB-78-Glioma | 0.0 | Raji-Burkitt's lymphoma | 0.0 |
| SF-268-Glioblastoma | 0.0 | Daudi-Burkitt's lymphoma | 0.0 |
| T98G-Glioblastoma | 0.0 | U266-B-cell plasmacytoma | 0.0 |
| SK-N-SH- | 0.0 | CA46-Burkitt's lymphoma | 0.0 |
| Neuroblastoma | | | |
| (metastasis) | | | |
| SF-295-Glioblastoma | 0.0 | RL-non-Hodgkin's B-cell | 0.0 |
| | | lymphoma | |
| Cerebellum | 0.0 | JM1-pre-B-cell lymphoma | 0.0 |
| Cerebellum | 0.0 | Jurkat-T cell leukemia | 0.0 |
| NCI-H292- | 0.0 | TF-1-Erythroleukemia | 0.0 |
| Mucoepidermoid lung | | | |
| carcinoma | | | |
| DMS-114-Small cell | 0.0 | HUT 78-T-cell lymphoma | 0.0 |
| lung cancer | | | |
| DMS-79-Small cell lung | 0.0 | U937-Histiocytic lymphoma | 0.0 |
| cancer | | | |
| NCI-H146-Small cell | 0.0 | KU-812-Myelogenous | 0.0 |
| lung cancer | | leukemia | |
| NCI-H526-Small cell | 0.0 | 769-P-Clear cell renal | 0.0 |
| lung cancer | | carcinoma | |
| NCI-N417-Small cell | 0.0 | Caki-2-Clear cell renal | 0.0 |
| lung cancer | | carcinoma | |
| NCI-H82-Small cell | 0.0 | SW 839-Clear cell renal | 0.0 |
| lung cancer | | carcinoma | |
| NCI-H157-Squamous | 0.0 | G401-Wilms' tumor | 0.0 |
| cell lung cancer | | | |
| (metastasis) | | | |
| NCI-H1155-Large cell | 0.0 | Hs766T-Pancreatic | 0.0 |
| lung cancer | | carcinoma (LN metastasis) | |
| NCI-H1299-Large cell | 0.0 | CAPAN-1-Pancreatic | 0.0 |
| lung cancer | | adenocarcinoma (liver | |
| | | metastasis) | |
| NCI-H727-Lung | 0.0 | SU86.86-Pancreatic | 0.0 |
| carcinoid | | carcinoma (liver metastasis) | |
| NCI-UMC-11-Lung | 0.0 | BxPC-3-Pancreatic | 0.0 |
| carcinoid | | adenocarcinoma | |
| LX-1-Small cell lung | 4.4 | HPAC-Pancreatic | 0.0 |
| cancer | | adenocarcinoma | |
| Colo-205-Colon cancer | 100.0 | MIA PaCa-2-Pancreatic | 0.0 |
| | | carcinoma | |
| KM12-Colon cancer | 0.0 | CFPAC-1-Pancreatic ductal | 0.0 |
| | | adenocarcinoma | |
| KM20L2-Colon cancer | 0.0 | PANC-1-Pancreatic | 0.0 |
| | | epithelioid ductal carcinoma | |
| NCI-H716-Colon cancer | 0.0 | T24-Bladder carcinma | 0.0 |
| | | (transitional cell) | |
| SW-48-Colon | 4.3 | 5637-Bladder carcinoma | 0.0 |
| adenocarcinoma | | | |
| SW1116-Colon | 0.0 | HT-1197-Bladder carcinoma | 0.0 |
| adenocarcinoma | | | |
| LS 174T-Colon | 10.1 | UM-UC-3-Bladder carcinma | 0.0 |
| adenocarcinoma | | (transitional cell) | |
| SW-948-Colon | 0.0 | A204-Rhabdomyosarcoma | 0.0 |
| adenocarcinoma | | | |
| SW-480-Colon | 0.0 | HT-1080-Fibrosarcoma | 0.0 |
| adenocarcinoma | | | |
| NCI-SNU-5-Gastric | 0.0 | MG-63-Osteosarcoma | 0.0 |
| carcinoma | | | |
| KATO III-Gastric | 1.2 | SK-LMS-1-Leiomyosarcoma | 0.0 |
| carcinoma | | (vulva) | |
| NCI-SNU-16-Gastric | 0.0 | SJRH30-Rhabdomyosarcoma | 0.7 |
| carcinoma | | (met to bone marrow) | |
| NCI-SNU-1-Gastric | 0.1 | A431-Epidermoid carcinoma | 0.0 |
| carcinoma | | | |
| RF-1-Gastric | 0.0 | WM266-4-Melanoma | 0.0 |
| adenocarcinoma | | | |
| RF-48-Gastric | 0.0 | DU 145-Prostate carcinoma | 0.0 |
| adenocarcinoma | | (brain metastasis) | |
| MKN-45-Gastric | 0.0 | MDA-MB-468-Breast | 0.0 |
| carcinoma | | adenocarcinoma | |
| NCI-N87-Gastric | 0.0 | SCC-4-Squamous cell | 0.0 |
| carcinoma | | carcinoma of tongue | |
| OVCAR-5-Ovarian | 0.0 | SCC-9-Squamous cell | 0.0 |
| carcinoma | | carcinoma of tongue | |
| RL95-2-Uterine | 0.0 | SCC-15-Squamous cell | 0.0 |
| carcinoma | | carcinoma of tongue | |
| HelaS3-Cervical | 0.0 | CAL 27-Squamous cell | 0.0 |
| adenocarcinoma | | carcinoma of tongue | |

TABLE BPF
Panel 4D

| | Rel. Exp. (%) | | Rel. Exp. (%) |
|---|---|---|---|
| | Ag3020, Run | | Ag3020, Run |
| Tissue Name | 164528102 | Tissue Name | 164528102 |
| Secondary Th1 act | 0.0 | HUVEC IL-1 beta | 0.0 |
| Secondary Th2 act | 0.0 | HUVEC IFN gamma | 0.0 |
| Secondary Tr1 act | 0.0 | HUVEC TNF alpha + IFN | 0.0 |
| | | gamma | |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC | 0.0 |
| | | none | |
| Primary Th1 act | 0.0 | Lung Microvascular EC | 0.0 |
| | | TNF alpha + IL-1 beta | |
| Primary Th2 act | 0.0 | Microvascular Dermal EC | 0.0 |
| | | none | |
| Primary Tr1 act | 0.0 | Microsvascular Dermal EC | 0.0 |
| | | TNF alpha + IL-1 beta | |
| Primary Th1 rest | 0.0 | Bronchial epithelium | 0.0 |
| | | TNF alpha + IL1 beta | |
| Primary Th2 rest | 0.0 | Small airway epithelium | 0.0 |
| | | none | |
| Primary Tr1 rest | 0.0 | Small airway epithelium | 0.0 |
| | | TNF alpha + IL-1 beta | |
| CD45RA CD4 | 0.0 | Coronery artery SMC rest | 0.0 |
| lymphocyte act | | | |
| CD45RO CD4 | 0.0 | Coronery artery SMC | 0.0 |
| lymphocyte act | | TNF alpha + IL-1 beta | |
| CD8 lymphocyte act | 0.0 | Astrocytes rest | 65.1 |
| Secondary CD8 | 0.0 | Astrocytes TNF alpha + | 21.0 |
| lymphocyte rest | | IL-1 beta | |
| Secondary CD8 | 0.0 | KU-812 (Basophil) rest | 10.7 |
| lymphocyte act | | | |
| CD4 lymphocyte none | 0.0 | KU-812 (Basophil) | 0.0 |
| | | PMA/ionomycin | |
| 2ry Th1/Th2/Tr1_anti- | 0.0 | CCD1106 (Keratinocytes) | 0.0 |
| CD95 CH11 | | none | |
| LAK cells rest | 13.3 | CCD1106 (Keratinocytes) | 0.0 |
| | | TNF alpha + IL-1 beta | |
| LAK cells IL-2 | 0.0 | Liver cirrhosis | 100.0 |
| LAK cells IL-2 + IL-12 | 0.0 | Lupus kidney | 0.0 |
| LAK cells IL-2 + IFN | 0.0 | NCI-H292 none | 0.0 |
| gamma | | | |
| LAK cells IL-2 + IL-18 | 0.0 | NCI-H292 IL-4 | 0.0 |
| LAK cells | 0.0 | NCI-H292 IL-9 | 0.0 |
| PMA/ionomycin | | | |
| NK Cells IL-2 rest | 8.0 | NCI-H292 IL-13 | 0.0 |
| Two Way MLR 3 day | 0.0 | NCI-H292 IFN gamma | 0.0 |
| Two Way MLR 5 day | 0.0 | HPAEC none | 0.0 |
| Two Way MLR 7 day | 10.6 | HPAEC TNF alpha + IL-1 | 11.2 |
| | | beta | |
| PBMC rest | 0.0 | Lung fibroblast none | 0.0 |
| PBMC PWM | 0.0 | Lung fibroblast TNF alpha + | 0.0 |
| | | IL-1 beta | |
| PBMC PHA-L | 0.0 | Lung fibroblast IL-4 | 0.0 |
| Ramos (B cell) none | 0.0 | Lung fibroblast IL-9 | 0.0 |
| Ramos (B cell) | 0.0 | Lung fibroblast IL-13 | 0.0 |
| ionomycin | | | |
| B lymphocytes PWM | 0.0 | Lung fibroblast IFN | 0.0 |
| | | gamma | |
| B lymphocytes CD40L | 9.9 | Dermal fibroblast | 0.0 |
| and IL-4 | | CCD1070 rest | |
| EOL-1 dbcAMP | 0.0 | Dermal fibroblast | 0.0 |
| | | CCD1070 TNF alpha | |
| EOL-1 dbcAMP | 0.0 | Dermal fibroblast | 0.0 |
| PMA/ionomycin | | CCD1070 IL-1 beta | |
| Dendritic cells none | 23.8 | Dermal fibroblast IFN | 0.0 |
| | | gamma | |
| Dendritic cells LPS | 0.0 | Dermal fibroblast IL-4 | 0.0 |
| Dendritic cells anti- | 0.0 | IBD Colitis 2 | 0.0 |
| CD40 | | | |
| Monycytes rest | 0.0 | IBD Crohn's | 0.0 |
| Monocytes LPS | 0.0 | Colon | 44.4 |
| Macrophages rest | 0.0 | Lung | 26.6 |
| Macrophages LPS | 0.0 | Thymus | 0.0 |
| HUVEC none | 0.0 | Kidney | 0.0 |
| HUVEC starved | 0.0 | | |

TABLE BPG
Panel 5D

| | Rel. | Rel. | | Rel. | Rel. |
|---|---|---|---|---|---|
| | Exp. (%) | Exp. (%) | | Exp. (%) | Exp. (%) |
| | Ag2968, | Ag3020, | | Ag2968, | Ag3020, |
| | Run | Run | | Run | Run |
| Tissue Name | 169270971 | 172171108 | Tissue Name | 169270971 | 172171108 |
| 97457_Patient- | 0.0 | 0.2 | 94709_Donor 2 AM - | 0.0 | 0.0 |
| 02go_adipose | | | A_adipose | | |
| 97476_Patient- | 1.2 | 8.4 | 94710_Donor 2 AM - | 0.0 | 0.0 |
| 07sk_skeletal | | | B_adipose | | |
| muscle | | | | | |
| 97477_Patient- | 0.1 | 0.0 | 94711_Donor 2 AM - | 0.0 | 0.0 |
| 07ut_uterus | | | C_adipose | | |
| 97478_Patient- | 0.0 | 1.3 | 94712_Donor 2 AD - | 0.0 | 3.0 |
| 07pl_placenta | | | A_adipose | | |
| 97481_Patient- | 2.8 | 12.6 | 94713_Donor 2 AD - | 100.0 | 0.0 |
| 08sk_skeletal | | | B_adipose | | |
| muscle | | | | | |
| 97482_Patient- | 0.0 | 0.0 | 94714_Donor 2 AD - | 0.0 | 0.0 |
| 08ut_uterus | | | C_adipose | | |
| 97483_Patient- | 0.0 | 0.0 | 94742_Donor 3 U - | 0.0 | 0.0 |
| 08pl_placenta | | | A_Mesenchymal Stem Cells | | |
| 97486_Patient- | 3.0 | 12.9 | 94743_Donor 3 U - | 0.0 | 0.0 |
| 09sk_skeletal | | | B_Mesenchymal Stem Cells | | |
| muscle | | | | | |
| 97487_Patient- | 0.1 | 0.7 | 94730_Donor 3 AM - | 0.0 | 0.0 |
| 09ut_uterus | | | A_adipose | | |
| 97488_Patient- | 0.1 | 0.2 | 94731_Donor 3 AM - | 0.0 | 0.0 |
| 09pl_placenta | | | B_adipose | | |
| 97492_Patient- | 0.0 | 0.2 | 94732_Donor 3 AM - | 0.0 | 0.0 |
| 10ut_uterus | | | C_adipose | | |
| 97493_Patient- | 0.2 | 1.1 | 94733_Donor 3 AD - | 0.0 | 0.0 |
| 10pl_placenta | | | A_adipose | | |
| 97495_Patient- | 0.0 | 0.0 | 94734_Donor 3 AD - | 0.0 | 0.0 |
| 11go_adipose | | | B_adipose | | |
| 97496_Patient- | 8.5 | 53.6 | 94735_Donor 3 AD - | 0.0 | 0.0 |
| 11sk_skeletal | | | C_adipose | | |
| muscle | | | | | |
| 97497_Patient- | 0.0 | 0.3 | 77138_Liver_HepG2untreated | 0.0 | 0.0 |
| 11ut_uterus | | | | | |
| 97498_Patient- | 0.1 | 2.3 | 73556_Heart_Cardiac stromal | 0.0 | 0.0 |
| 11pl_placenta | | | cells (primary) | | |
| 97500_Patient- | 0.0 | 0.4 | 81735_Small Intestine | 0.1 | 0.0 |
| 12go_adipose | | | | | |
| 97501_Patient- | 19.1 | 100.0 | 72409_Kidney_Proximal | 0.0 | 0.0 |
| 12sk_skeletal | | | Convoluted Tubule | | |
| muscle | | | | | |
| 97502_Patient- | 0.1 | 0.0 | 82685_Small | 0.0 | 0.0 |
| 12ut_uterus | | | intestine_Duodenum | | |
| 97503_Patient- | 0.1 | 1.3 | 90650_Adrenal_Adrenocortical | 0.0 | 0.2 |
| 12pl_placenta | | | adenoma | | |
| 94721_Donor 2 | 0.0 | 0.0 | 72410_Kidney_HRCE | 0.0 | 0.3 |
| U - | | | | | |
| A_Mesenchymal | | | | | |
| Stem Cells | | | | | |
| 94722_Donor 2 | 0.0 | 0.0 | 72411_Kidney_HRE | 0.0 | 0.0 |
| U - | | | | | |
| B_Mesenchymal | | | | | |
| Stem Cells | | | | | |
| 94723_Donor 2 | 0.0 | 0.0 | 73139_Uterus_Uterine smooth | 0.0 | 0.0 |
| U - | | | muscle cells | | |
| C_Mesenchymal | | | | | |
| Stem Cells | | | | | |

General_screening_panel_v1.4 Summary: Ag3020 The NOV75 gene is expressed in brain, colon, lung, and ovarian cancer cell lines with highest expression in a colon cancer cell line Colo-205 (CT=24.37). This suggests that this gene can be used as a diagnostic marker for these types of cancer. Furthermore, inhibition of the protein using small molecule drugs could potentially be useful for treatment of brain, colon, lung and ovarian cancer.

In addition, this gene has low expression in adipose and high expression in adult and fetal heart and skeletal muscle. Thus, this protein phosphatase may be a small molecule target for the treatment of obesity, Type 2 diabetes and cardiac and skeletal muscle disease.

Pane 1.3D Summary: Ag2968/Ag3020 Results from two experiments using identical probe/primer sets are in excellent agreement. Expression of the NOV75 gene is highest in adult skeletal muscle (CTs=26-28). Significant but somewhat lower expression is also seen in fetal skeletal muscle and adult/fetal heart. This, expression of this gene may be used to distinguish these samples from the other samples on this panel.

This gene is also expressed in brain, colon, lung and ovarian cancer cell lines, consistent with General_screening_panel_v1.4. This suggests that this gene can be used as a diagnostic marker for these types of cancer and inhibition of the protein using small molecule drugs can be used for treatment of brain, colon, lung and ovarian cancer.

Panel 3D Summary: Ag2968 Expression of the NOV75 gene is highest in colon cancer cell lines, consistent with General_screening_panel_v1.4. This suggests that this gene can be used as a diagnostic marker for these types of cancer and inhibition of the protein using small molecule drugs can be used for the treatment of brain, colon, lung and ovarian cancer.

Panel 3D Summary: Ag2968 Expression of the NOV75 gene is highest in colon cancer cell line Colo-205 (CT=25.6). In addition, significant expression of this gene is seen in two other colon cancer cell lines. Thus, expression of this gene may be used to distinguish these colon cancer cell lines from the other samples on this panel. Moreover, therapeutic modulation of the activity of this gene or its protein product, using small molecules, antibodies or protein therapeutics, may be benefit in the treatment of colon cancer.

Panel 4D Summary: Ag3020 Expression of the NOV75 gene is highest in a liver cirrhosis sample (CT=33.3). Furthermore, expression of this gene is not detected in normal livers in Panels 1.3D or 1.4, suggesting that its expression is unique to liver cirrhosis. This gene encodes a putative protein phosphatase; therefore, antibodies or small molecule therapeutics could reduce or inhibit fibrosis that occurs in liver cirrhosis. In addition protein could also be used for the diagnosis of liver cirrhosis. Low levels of expression are also seen in colon and resting astrocytes.

Ag2968 Expression of this gene is low/undetectable (CTs>35) across all of the samples on this panel (data not shown).

Panel 5D Summary: Ag3020 Expression of the NOV75 gene is primarily restricted to samples from skeletal muscle. This specific expression is in agreement with the results in Panels 1.3d and 1.4. Thus, expression of this gene could be used to differentiate between skeletal muscle and other samples on the panel, and as a marker of skeletal muscle.

NOV6a

Expression of gene NOV76a was assessed using the primer-probe sets Ag3022 and Ag4891, described in Tables BQA and BQB. Results of the RTQ-PCR runs are shown in Tables BQC and BQD.

TABLE BQA
Probe Name Ag3022

| | | | Start | |
|---|---|---|---|---|
| Primers | Sequences | Length | Position | SEQ ID NO: |
| Forward | 5′-tgcttgcaggtttattcttagg-3′ | 22 | 431 | 1297 |
| Probe | TET-5′-tgctgagttctctcgttgtttccctg-3′-TAMRA | 26 | 459 | 1298 |
| Reverse | 5′-tgagaaatgcaggtagggacta-3′ | 22 | 509 | 1299 |

TABLE BQB
Probe Name Ag4891

| | | | Start | |
|---|---|---|---|---|
| Primers | Sequences | Length | Position | SEQ ID NO: |
| Forward | 5′-aatacctgtccaaagcctgact-3′ | 22 | 661 | 1300 |
| Probe | TET-5′-ttatccccgagtctcatttcctgcgt-3′-TAMRA | 26 | 683 | 1301 |
| Reverse | 5′-ttctcacaaaagctgtcattca-3′ | 22 | 716 | 1302 |

TABLE BQC
General_screening_panel_v1.5

| | Rel. Exp. (%) Ag4891, | | Rel. Exp. (%) Ag4891, |
|---|---|---|---|
| Tissue Name | Run 228714709 | Tissue Name | Run 228714709 |
| Adipose | 10.8 | Renal ca. TK-10 | 24.5 |
| Melanoma* | 5.6 | Bladder | 22.8 |
| Hs688(A).T | | | |
| Melanoma* | 6.3 | Gastric ca. (liver met.) | 46.7 |
| Hs688(B).T | | NCI-N87 | |
| Melanoma* M14 | 27.4 | Gastric ca. KATO III | 22.2 |
| Melanoma* | 9.5 | Colon ca. SW-948 | 11.5 |
| LOXIMVI | | | |
| Melanoma* SK- | 12.4 | Colon ca. SW480 | 20.7 |
| MEL-5 | | | |
| Squamous cell | 10.2 | Colon ca.* (SW480 | 11.6 |
| carcinoma SCC-4 | | met) SW620 | |
| Testis Pool | 11.3 | Colon ca. HT29 | 10.6 |
| Prostate ca.* (bone | 6.7 | Colon ca. HCT-116 | 12.2 |
| met) PC-3 | | | |
| Prostate Pool | 16.8 | Colon ca. CaCo-2 | 73.2 |
| Placenta | 33.7 | Colon cancer tissue | 45.4 |
| Uterus Pool | 9.7 | Colon ca. SW1116 | 3.0 |
| Ovarian ca. | 30.4 | Colon ca. Colo-205 | 5.4 |
| OVCAR-3 | | | |
| Ovarian ca. SK-OV-3 | 10.2 | Colon ca. SW-48 | 10.2 |
| Ovarian ca. | 8.5 | Colon Pool | 15.9 |
| OVCAR-4 | | | |
| Ovarian ca. | 21.8 | Small Intestine Pool | 13.2 |
| OVCAR-5 | | | |
| Ovarian ca. IGROV-1 | 10.6 | Stomach Pool | 7.4 |
| Ovarian ca. | 5.6 | Bone Marrow Pool | 5.7 |
| OVCAR-8 | | | |
| Ovary | 6.5 | Fetal Heart | 11.3 |
| Breast ca. MCF-7 | 52.5 | Heart Pool | 7.0 |
| Breast ca. MDA- | 11.5 | Lymph Node Pool | 18.4 |
| MB-231 | | | |
| Breast ca. BT 549 | 41.8 | Fetal Skeletal Muscle | 9.2 |
| Breast ca. T47D | 4.4 | Skeletal Muscle Pool | 16.3 |
| Breast ca. MDA-N | 12.1 | Spleen Pool | 19.3 |
| Breast Pool | 17.8 | Thymus Pool | 14.7 |
| Trachea | 12.1 | CNS cancer (glio/astro) | 12.2 |
| | | U87-MG | |
| Lung | 4.7 | CNS cancer (glio/astro) | 11.8 |
| | | U-118-MG | |
| Fetal Lung | 36.3 | CNS cancer | 24.5 |
| | | (neuro; met) SK-N-AS | |
| Lung ca. NCI-N417 | 4.4 | CNS cancer (astro) SF- | 5.4 |
| | | 539 | |
| Lung ca. LX-1 | 9.9 | CNS cancer (astro) | 13.0 |
| | | SNB-75 | |
| Lung ca. NCI-H146 | 15.0 | CNS cancer (glio) | 11.7 |
| | | SNB-19 | |
| Lung ca. SHP-77 | 6.8 | CNS cancer (glio) SF- | 15.7 |
| | | 295 | |
| Lung ca. A549 | 28.7 | Brain (Amygdala) Pool | 10.8 |
| Lung ca. NCI-H526 | 0.8 | Brain (cerebellum) | 35.8 |
| Lung ca. NCI-H23 | 38.4 | Brain (fetal) | 9.3 |
| Lung ca. NCI-H460 | 10.0 | Brain (Hippocampus) | 10.2 |
| | | Pool | |
| Lung ca. HOP-62 | 11.7 | Cerebral Cortex Pool | 9.7 |
| Lung ca. NCI-H522 | 29.5 | Brain (Substantia nigra) | 10.3 |
| | | Pool | |
| Liver | 6.1 | Brain (Thalamus) Pool | 15.1 |
| Fetal Liver | 19.3 | Brain (whole) | 10.3 |
| Liver ca. HepG2 | 34.4 | Spinal Cord Pool | 10.3 |
| Kidney Pool | 23.7 | Adrenal Gland | 100.0 |
| Fetal Kidney | 11.6 | Pituitary gland Pool | 5.3 |
| Renal ca. 786-0 | 14.6 | Salivary Gland | 12.5 |
| Renal ca. A498 | 5.4 | Thyroid (female) | 6.7 |
| Renal ca. ACHN | 21.6 | Pancreatic ca. CAPAN2 | 21.0 |
| Renal ca. UO-31 | 25.9 | Pancreas Pool | 25.9 |

TABLE BQD
Panel 4.1D

| | Rel. Exp. (%) | | Rel. Exp. (%) |
|---|---|---|---|
| | Ag4891, Run | | Ag4891, Run |
| Tissue Name | 214253687 | Tissue Name | 214253687 |
| Secondary Th1 act | 55.1 | HUVEC IL-1beta | 17.7 |
| Secondary Th2 act | 100.0 | HUVEC IFN gamma | 6.2 |
| Secondary Tr1 act | 74.2 | HUVEC TNF alpha + IFN | 9.0 |
| | | gamma | |
| Secondary Th1 rest | 18.8 | HUVEC TNF alpha + IL4 | 11.7 |
| Secondary Th2 rest | 34.4 | HUVEC IL-11 | 2.9 |
| Secondary Tr1 rest | 20.9 | Lung Microvascular EC | 21.3 |
| | | none | |
| Primary Th1 act | 18.8 | Lung Microvascular EC | 26.2 |
| | | TNF alpha + IL-1beta | |
| Primary Th2 act | 60.3 | Microvascular Dermal EC | 6.7 |
| | | none | |
| Primary Tr1 act | 35.4 | Microsvasular Dermal EC | 15.1 |
| | | TNF alpha + IL-1beta | |
| Primary Th1 rest | 17.7 | Bronchial epithelium | 16.8 |
| | | TNF alpha + IL1beta | |
| Primary Th2 rest | 24.8 | Small airway epithelium | 4.0 |
| | | none | |
| Primary Tr1 rest | 14.8 | Small airway epithelium | 13.3 |
| | | TNF alpha + IL-1beta | |
| CD45RA CD4 | 15.1 | Coronery artery SMC rest | 8.4 |
| lymphocyte act | | | |
| CD45RO CD4 | 63.3 | Coronery artery SMC | 8.2 |
| lymphocyte act | | TNF alpha + IL-1beta | |
| CD8 lymphocyte act | 23.0 | Astrocytes rest | 8.4 |
| Secondary CD8 | 46.0 | Astrocytes TNF alpha + | 3.1 |
| lymphocyte rest | | IL-1beta | |
| Secondary CD8 | 18.6 | KU-812 (Basophil) rest | 0.5 |
| lymphocyte act | | | |
| CD4 lymphocyte none | 15.5 | KU-812 (Basophil) | 2.3 |
| | | PMA/ionomycin | |
| 2ry Th1/Th2/Tr1_anti- | 47.0 | CCD1106 (Keratinocytes) | 3.3 |
| CD95 CH11 | | none | |
| LAK cells rest | 15.4 | CCD1106 (Keratinocytes) | 10.7 |
| | | TNF alpha + IL-1beta | |
| LAK cells IL-2 | 30.4 | Liver cirrhosis | 13.7 |
| LAK cells IL-2 + IL-12 | 12.9 | NCI-H292 none | 28.9 |
| LAK cells IL-2 + IFN | 12.8 | NCI-H292 IL-4 | 24.0 |
| gamma | | | |
| LAK cells IL-2 + IL-18 | 22.4 | NCI-H292 IL-9 | 52.9 |
| LAK cells | 24.8 | NCI-H292 IL-13 | 26.6 |
| PMA/ionomycin | | | |
| NK Cells IL-2 rest | 20.0 | NCI-H292 IFN gamma | 30.8 |
| Two Way MLR 3 day | 29.7 | HPAEC none | 4.2 |
| Two Way MLR 5 day | 32.5 | HPAEC TNF alpha + IL-1 | 58.2 |
| | | beta | |
| Two Way MLR 7 day | 42.6 | Lung fibroblast none | 10.0 |
| PBMC rest | 10.3 | Lung fibroblast TNF | 7.5 |
| | | alpha + IL-1beta | |
| PBMC PWM | 47.0 | Lung fibroblast IL-4 | 4.5 |
| PBMC PHA-L | 49.7 | Lung fibroblast IL-9 | 5.9 |
| Ramos (B cell) none | 1.6 | Lung fibroblast IL-13 | 3.4 |
| Ramos (B cell) | 1.5 | Lung fibroblast IFN | 8.4 |
| ionomycin | | gamma | |
| B lymphocytes PWM | 28.1 | Dermal fibroblast | 4.1 |
| | | CCD1070 rest | |
| B lymphocytes CD40L | 11.4 | Dermal fibroblast | 42.9 |
| and IL-4 | | CCD1070 TNF alpha | |
| EOL-1 dbcAMP | 1.5 | Dermal fibroblast | 4.9 |
| | | CCD1070 IL-1beta | |
| EOL-1 dbcAMP | 1.9 | Dermal fibroblast IFN | 7.3 |
| PMA/ionomycin | | gamma | |
| Dendritic cells none | 10.2 | Dermal fibroblast IL-4 | 6.8 |
| Dendritic cells LPS | 7.3 | Dermal Fibroblasts rest | 7.7 |
| Dendritic cells anti- | 6.0 | Neutrophils TNFa + LPS | 13.5 |
| CD40 | | | |
| Monocytes rest | 2.5 | Neutrophils rest | 12.8 |
| Monocytes LPS | 34.9 | Colon | 6.0 |
| Macrophages rest | 9.4 | Lung | 10.6 |
| Macrophages LPS | 7.1 | Thymus | 20.3 |
| HUVEC none | 5.4 | Kidney | 10.2 |
| HUVEC starved | 8.5 | | |

CNS_neurodegeneration_v1.0 Summary: Ag3022 No significant expression detected. The amp plot indicates that there is a possibility of a potential chemistry or probe/primer failure (data not shown).

General_screening_panel_v1.4 Summary: Ag3022 The amp plot indicates that there is possibility of a potential chemistry or probe/primer failure (data not shown).

General_screening_panel_v1.5 Summary: Ag4891 the NOV6a gene has moderate levels of expression in adipose, liver, heart, skeletal muscle, pituitary, thyroid and pancreas, and high levels of expression in adrenal gland. This, this gene product may be a small molecule target for the treatment of metabolic, endocrine and adrenal diseases, including obesity, Types 1 and 2 diabetes, and Addison's disease.

In addition, this gene is expressed at moderate levels in the cancer cell lines in this panel. A higher level of expression is observed in colon, lung, breast and ovarian cancer cell lines when compared to samples from the normal colon, lung, breast and ovary. Thus, this gene could be used as a diagnostic marker of cancer in these tissues. Furthermore, inhibition of the activity of this gene product using small molecule drugs may be useful for the treatment of cancer in these tissues.

This gene encodes a homolog of a dual specificity phosphotase that is also expressed at low to moderate levels across the CNS. Dual-specificity phosphatases comprise a family of MAP kinase regulating enzymes, members of which are upregulated in brains subjected to insults such as ischemia and seizure activity. MAP kinases are known to regulate neurotrophic and neurotoxic pathways. Consequently, agents that modulate the activity of this gene may have utility in attenuating the apoptotic and neurodegenerative processes following brain insults.

References:

Wiessner C. The dual specificity phosphatase PAC-1 is transcriptionally induced in the rat brain following transient forebrain ischemia. Brain Res Mol Brain Res February 1995;28(2):353-6

PAC-1 mRNA has previously been found only in activated T-cells in vitro and in vivo. The gene encodes a dual specificity protein phosphatase that regulates MAP kinase activity. Here, I describe that PAC-1 mRNA is induced also in neurons in the rat brain following 30 min of forebrain ischemia. At 6, 12 and 24 h after ischemia, PAC-1 mRNA was found most prominently in hippocampal cells which are resistant to 30 min of forebrain ischemia, but not in the selectively vulnerable CA1 sector. At later time points and in control animals no PAC-1 mRNA could be detected in any brain region. The protein-tyrosine/threonine phospatase PAC-1, therefore, may be involved in adaptational responses of hippocampal cells resistant to ischemic injury.

Boschert U, Muda M, Camps M, Dickinson R, Arkinstall S. Induction of the dual specificity phosphatase PAC1 in rat brain following seizure activity. Neuroreport Sep. 29, 1997:8(14):3077-80

Recurrent seizure activity leads to delayed neuronal death as well as to inflammatory responses involving microglia in hippocampal subfields CA1, CA3 and CA4. Since mitogen activated protein (MAP) kinases control neuronal apoptosis and trigger generation of inflammatory cytokines, their activation state could determine seizure-related brain damage. PAC1 is a dual specificity protein phosphatase inactivating MAP kinases which we have found to be undetectable in normal brain. Despite this, kainic acid-induced seizure activity leads to rapid (approximately 3 h) but transient appearance of PAC1 mRNA in granule cells of the dentate gyrus as well as in pyramidal CA1 neurons. This pattern changed with time and after 2-3 days PAC1 was induced in dying CA1 and CA3 neurons. At this time PAC1 mRNA was also expressed in white matter microglia as well as microglia invading the damaged hippocampus. PAC1 may play an important role controlling MAP kinase involvement in both neuronal death and neuro-inflammation following excitotoxic damage.

Panel 4.1D Summary: Ag4891 The NOV76a gene is expressed in a wide range of cell types and tissues (CT=26-34) of significance in the immune response in health and disease. Highest expression of this gene is detected in activated secondary Th2 cells (CT=26.48). Therefore, targeting of this gene product with a small molecule drug or antibody therapeutic may modulate the functions of cells of the immune system as well as resident tissue cells and lead to improvement of the symptoms of patients suffering from autoimmune and inflammatory diseases such as asthma, allergies, inflammatory bowel disease, lupus erythematosus, and arthritis, including osteoarthritis and rheumatoid arthritis.

Panel 4D Summary: Ag3022 No significant expression detected. Potential probe/primer failure (data not shown).

NOV77

Expression of gene NOV77 was assessed using the primer-probe sets Ag3023 and Ag3373, described in Tables BRA and BRB. Results of the RTQ-PCR runs are shown in Tables BRC, BRD, BRE and BRF.

TABLE BRA
Probe Name Ag3023

| | | | | |
|---|---|---|---|---|
| | | | Start | |
| Primers | Sequences | Length | Position | SEQ ID NO: |

| | | | | |
|---|---|---|---|---|
| Forward | 5′-ctaatgctggatttgtccatca-3′ | 22 | 572 | 1303 |
| Probe | TET-5′-tcaggaatatgaagccatctacctagca-3′-TAMRA | 28 | 597 | 1304 |
| Reverse | 5′-tggagtggtgacatctgta-3′ | 22 | 635 | 1305 |

TABLE BRB
Probe Name Ag3373

| | | | Start | |
|---|---|---|---|---|
| Primers | Sequences | Length | Position | SEQ ID NO: |
| Forward | 5′-atttgtccatcaacttcaggaa-3′ | 22 | 582 | 1306 |
| Probe | TET-5′-tgaagccatctacctagcaaaattaaca-3′-TAMRA | 28 | 606 | 1307 |
| Reverse | 5′-tggagtggtgacatcatctgta-3′ | 22 | 635 | 1308 |

TABLE BRC
CNS_neurodegeneration_v1.0

| | Rel. Exp. (%) | Rel. Exp. (%) | | Rel. Exp. (%) | Rel. Exp. (%) |
|---|---|---|---|---|---|
| | Ag3023, Run | Ag3373, Run | Tissue | Ag3023, Run | Ag3373, Run |
| Tissue Name | 209821074 | 210154071 | Name | 209821074 | 210154071 |
| AD 1 Hippo | 10.9 | 16.8 | Control | 9.1 | 8.0 |
| | | | (Path) 3 | | |
| | | | Temporal | | |
| | | | Ctx | | |
| AD 2 Hippo | 34.2 | 37.6 | Control | 40.6 | 65.5 |
| | | | (Path) 4 | | |
| | | | Temporal | | |
| | | | Ctx | | |
| AD 3 Hippo | 12.0 | 15.8 | AD 1 | 24.7 | 29.1 |
| | | | Occipital | | |
| | | | Ctx | | |
| AD 4 Hippo | 13.8 | 10.3 | AD 2 | 0.0 | 0.0 |
| | | | Occipital | | |
| | | | Ctx | | |
| | | | (Missing) | | |
| AD 5 hippo | 60.7 | 57.8 | AD 3 | 14.7 | 15.0 |
| | | | Occipital | | |
| | | | Ctx | | |
| AD 6 Hippo | 80.7 | 72.2 | AD 4 | 35.4 | 22.4 |
| | | | Occipital | | |
| | | | Ctx | | |
| Control 2 | 35.8 | 38.4 | AD 5 | 3.9 | 30.4 |
| Hippo | | | Occipital | | |
| | | | Ctx | | |
| Control 4 | 16.5 | 11.7 | AD 6 | 46.0 | 37.4 |
| Hippo | | | Occipital | | |
| | | | Ctx | | |
| Control (Path) | 13.1 | 15.4 | Control 1 | 9.9 | 10.7 |
| 3 Hippo | | | Occipital | | |
| | | | Ctx | | |
| AD 1 Temporal | 39.0 | 31.4 | Control 2 | 39.0 | 38.4 |
| Ctx | | | Occipital | | |
| | | | Ctx | | |
| AD 2 Temporal | 38.7 | 73.2 | Control 3 | 23.0 | 20.6 |
| Ctx | | | Occipital | | |
| | | | Ctx | | |
| AD 3 Temporal | 9.5 | 13.2 | Control 4 | 13.3 | 13.3 |
| Ctx | | | Occipital | | |
| | | | Ctx | | |
| AD 4 Temporal | 27.9 | 34.9 | Control | 80.1 | 76.3 |
| Ctx | | | (Path) 1 | | |
| | | | Occipital | | |
| | | | Ctx | | |
| AD 5 Inf | 59.0 | 100.0 | Control | 17.3 | 20.0 |
| Temporal Ctx | | | (Path) 2 | | |
| | | | Occipital | | |
| | | | Ctx | | |
| AD 5 | 33.2 | 44.1 | Control | 8.4 | 8.7 |
| SupTemporal | | | (Path) 3 | | |
| Ctx | | | Occipital | | |
| | | | Ctx | | |
| AD 6 Inf | 100.0 | 73.2 | Control | 21.2 | 20.6 |
| Temporal Ctx | | | (Path) 4 | | |
| | | | Occipital | | |
| | | | Ctx | | |
| AD 6 Sup | 79.6 | 80.1 | Control 1 | 12.1 | 16.3 |
| Temporal Ctx | | | Parietal Ctx | | |
| Control 1 | 10.2 | 13.7 | Control 2 | 48.0 | 40.9 |
| Temporal Ctx | | | Parietal Ctx | | |
| Control 2 | 41.2 | 31.9 | Control 3 | 17.9 | 16.3 |
| Temporal Ctx | | | Parietal Ctx | | |
| Control 3 | 20.3 | 20.0 | Control | 74.7 | 64.2 |
| Temporal Ctx | | | (Path) 1 | | |
| | | | Parietal Ctx | | |
| Control 4 | 9.7 | 9.9 | Control | 28.9 | 59.9 |
| Temporal Ctx | | | (Path) 2 | | |
| | | | Parietal Ctx | | |
| Control (Path) | 59.9 | 68.3 | Control | 10.2 | 9.0 |
| 1 Temporal Ctx | | | (Path) 3 | | |
| | | | Parietal Ctx | | |
| Control (Path) | 40.3 | 41.2 | Control | 44.8 | 43.8 |
| 2 Temporal Ctx | | | (Path) 4 | | |
| | | | Parietal Ctx | | |

TABLE BRD
General_screening_panel_v1.4

| | Rel. Exp. (%) Ag3373, | | Rel. Exp. (%) Ag3373, |
|---|---|---|---|
| Tissue Name | Run 217043119 | Tissue Name | Run 217043119 |
| Adipose | 12.0 | Renal ca. TK-10 | 20.3 |
| Melanoma* | 30.8 | Bladder | 23.2 |
| Hs688(A).T | | | |
| Melanoma* | 69.3 | Gastric ca. (liver met.) | 25.3 |
| Hs688(B).T | | NCI-N87 | |
| Melanoma* M14 | 15.0 | Gastric ca. KATO III | 30.8 |
| Melanoma* | 26.6 | Colon ca. SW-948 | 9.7 |
| LOXIMVI | | | |
| Melanoma* SK- | 21.5 | Colon ca. SW480 | 35.1 |
| MEL-5 | | | |
| Squamous cell | 33.0 | Colon ca.* (SW480 | 13.9 |
| carcinoma SCC-4 | | met) SW620 | |
| Testis Pool | 19.8 | Colon ca. HT29 | 8.5 |
| Prostate ca.* (bone | 100.0 | Colon ca. HCT-116 | 36.9 |
| met) PC-3 | | | |
| Prostate Pool | 9.2 | Colon ca. CaCo-2 | 42.9 |
| Placenta | 3.8 | Colon cancer tissue | 9.0 |
| Uterus Pool | 7.4 | Colon ca. SW1116 | 5.8 |
| Ovarian ca. | 28.5 | Colon ca. Colo-205 | 4.3 |
| OVCAR-3 | | | |
| Ovarian ca. SK-OV-3 | 40.3 | Colon ca. SW-48 | 4.2 |
| Ovarian ca. | 20.0 | Colon Pool | 20.7 |
| OVCAR-4 | | | |
| Ovarian ca. | 35.1 | Small Intestine Pool | 12.2 |
| OVCAR-5 | | | |
| Ovarian ca. IGROV-1 | 10.9 | Stomach Pool | 9.9 |
| Ovarian ca. | 9.2 | Bone Marrow Pool | 11.6 |
| OVCAR-8 | | | |
| Ovary | 9.7 | Fetal Heart | 20.7 |
| Breast ca. MCF-7 | 37.6 | Heart Pool | 10.6 |
| Breast ca. MDA- | 37.1 | Lymph Node Pool | 17.9 |
| MB-231 | | | |
| Breast ca. BT 549 | 62.4 | Fetal Skeletal Muscle | 12.3 |
| Breast ca. T47D | 61.1 | Skeletal Muscle Pool | 16.0 |
| Breast ca. MDA-N | 10.0 | Spleen Pool | 11.6 |
| Breast Pool | 17.3 | Thymus Pool | 12.2 |
| Trachea | 12.0 | CNS cancer (glio/astro) | 29.1 |
| | | U87-MG | |
| Lung | 6.7 | CNS cancer (glio/astro) | 69.3 |
| | | U-118-MG | |
| Fetal Lung | 34.2 | CNS cancer | 34.9 |
| | | (neuro; met) SK-N-AS | |
| Lung ca. NCI-N417 | 5.4 | CNS cancer (astro) SF- | 19.1 |
| | | 539 | |
| Lung ca. LX-1 | 17.2 | CNS cancer (astro) | 35.8 |
| | | SNB-75 | |
| Lung ca. NCI-H146 | 3.0 | CNS cancer (glio) | 11.3 |
| | | SNB-19 | |
| Lung ca. SHP-77 | 18.6 | CNS cancer (glio) SF- | 26.4 |
| | | 295 | |
| Lung ca. A549 | 29.1 | Brain (Amygdala) Pool | 4.5 |
| Lung ca. NCI-H526 | 4.6 | Brain (cerebellum) | 8.1 |
| Lung ca. NCI-H23 | 31.6 | Brain (fetal) | 13.2 |
| Lung ca. NCI-H460 | 18.2 | Brain (Hippocampus) | 5.3 |
| | | Pool | |
| Lung ca. HOP-62 | 14.1 | Cerebral Cortex Pool | 5.4 |
| Lung ca. NCI-H522 | 31.6 | Brain (Substantia nigra) | 4.8 |
| | | Pool | |
| Liver | 1.2 | Brain (Thalamus) Pool | 8.0 |
| Fetal Liver | 32.3 | Brain (whole) | 6.2 |
| Liver ca. HepG2 | 14.6 | Spinal Cord Pool | 6.6 |
| Kidney Pool | 22.1 | Adrenal Gland | 8.1 |
| Fetal Kidney | 26.1 | Pituitary gland Pool | 3.0 |
| Renal ca. 786-0 | 28.7 | Salivary Gland | 4.7 |
| Renal ca. A498 | 11.3 | Thyroid (female) | 4.4 |
| Renal ca. ACHN | 12.2 | Pancreatic ca. CAPAN2 | 17.3 |
| Renal ca. UO-31 | 24.1 | Pancreas Pool | 17.1 |

TABLE BRE
Panel 1.3D

| | Rel. Exp. (%) Ag3023, | | Rel. Exp. (%) Ag3023, |
|---|---|---|---|
| Tissue Name | Run 167966931 | Tissue Name | Run 167966931 |
| Liver adenocarcinoma | 51.1 | Kidney (fetal) | 26.2 |
| Pancreas | 6.1 | Renal ca. 786-0 | 34.2 |
| Pancreatic ca. CAPAN 2 | 17.7 | Renal ca. A498 | 17.6 |
| Adrenal gland | 3.8 | Renal ca. RXF 393 | 17.2 |
| Thyroid | 3.0 | Renal ca. ACHN | 13.5 |
| Salivary gland | 3.9 | Renal ca. UO-31 | 0.0 |
| Pituitary gland | 3.6 | Renal ca. TK-10 | 23.0 |
| Brain (fetal) | 8.1 | Liver | 11.7 |
| Brain (whole) | 8.5 | Liver (fetal) | 8.0 |
| Brain (amygdala) | 6.7 | Liver ca. | 26.2 |
| | | (hepatoblast) HepG2 | |
| Brain (cerebellum) | 15.2 | Lung | 3.1 |
| Brain (hippocampus) | 5.4 | Lung (fetal) | 11.0 |
| Brain (substantia nigra) | 9.0 | Lung ca. (small cell) | 12.9 |
| | | LX-1 | |
| Brain (thalamus) | 4.2 | Lung ca. (small cell) | 9.9 |
| | | NCI-H69 | |
| Cerebral Cortex | 2.0 | Lung ca. (s.cell var.) | 67.8 |
| | | SHP-77 | |
| Spinal cord | 6.9 | Lung ca. (large | 3.4 |
| | | cell) NCI-H460 | |
| glio/astro U87-MG | 28.5 | Lung ca. (non-sm. | 45.1 |
| | | cell) A549 | |
| glio/astro U-118-MG | 46.7 | Lung ca. (non-s.cell) | 22.7 |
| | | NCI-H23 | |
| astro cytoma SW1783 | 40.6 | Lung ca. (non-s.cell) | 25.7 |
| | | HOP-62 | |
| neuro*; met SK-N-AS | 27.2 | Lung ca. (non-s.cl) | 38.2 |
| | | NCI-H522 | |
| astrocytoma SF-539 | 29.7 | Lung ca. (squam.) | 27.4 |
| | | SW 900 | |
| astrocytoma SNB-75 | 35.1 | Lung ca. (squam.) | 29.9 |
| | | NCI-H596 | |
| glioma SNB-19 | 15.6 | Mammary gland | 5.1 |
| glioma U251 | 37.9 | Breast ca.* (pl.ef) | 47.0 |
| | | MCF-7 | |
| glioma SF-295 | 18.4 | Breast ca.* (pl.ef) | 22.7 |
| | | MDA-MB-231 | |
| Heart (fetal) | 2.9 | Breast ca.* (pl.ef) | 86.5 |
| | | T47D | |
| Heart | 12.9 | Breast ca. BT-549 | 15.9 |
| Skeletal muscle (fetal) | 3.4 | Breast ca. MDA-N | 10.4 |
| Skeletal muscle | 36.3 | Ovary | 2.9 |
| Bone marrow | 4.5 | Ovarian ca. OVCAR-3 | 26.1 |
| Thymus | 14.3 | Ovarian ca. OVCAR-4 | 16.3 |
| Spleen | 8.7 | Ovarian ca. OVCAR-5 | 83.5 |
| Lymph node | 11.8 | Ovarian ca. OVCAR-8 | 9.3 |
| Colorectal | 10.4 | Ovarian ca. IGROV-1 | 12.0 |
| Stomach | 7.8 | Ovarian ca.* (ascites) | 100.0 |
| | | SK-OV-3 | |
| Small intestine | 5.1 | Uterus | 4.9 |
| Colon ca. SW480 | 19.3 | Placenta | 1.3 |
| Colon ca.* | 42.9 | Prostate | 3.9 |
| SW620 (SW480 met) | | | |
| Colon ca. HT29 | 9.9 | Prostate ca.* (bone | 78.5 |
| | | met)PC-3 | |
| Colon ca. HCT-116 | 26.2 | Testis | 9.7 |
| Colon ca. CaCo-2 | 41.5 | Melanoma | 5.9 |
| | | Hs688(A).T | |
| Colon ca. | 6.3 | Melanoma* (met) | 14.2 |
| tissue (ODO3866) | | Hs688(B).T | |
| Colon ca. HCC-2998 | 16.0 | Melanoma UACC-62 | 14.0 |
| Gastric ca.* (liver met) | 18.8 | Melanoma M14 | 5.7 |
| NCI-N87 | | | |
| Bladder | 30.6 | Melanoma LOX | 8.8 |
| | | IMVI | |
| Trachea | 3.2 | Melanoma* (met) | 14.7 |
| | | SK-MEL-5 | |
| Kidney | 9.6 | Adipose | 18.9 |

TABLE BRF
Panel 4D

| | Rel. | Rel. | | Rel. | Rel. |
|---|---|---|---|---|---|
| | Exp. (%) | Exp. (%) | | Exp. (%) | Exp. (%) |
| | Ag3023, | Ag3373, | | Ag3023, | Ag3373, |
| | Run | Run | | Run | Run |
| Tissue Name | 164516146 | 165296617 | Tissue Name | 164516146 | 165296617 |
| Secondary Th1 act | 18.6 | 17.9 | HUVEC IL-1beta | 20.3 | 18.6 |
| Secondary Th2 act | 24.3 | 28.5 | HUVEC IFN | 25.3 | 22.7 |
| | | | gamma | | |
| Secondary Tr1 act | 22.8 | 21.8 | HUVEC TNF | 16.3 | 18.0 |
| | | | alpha + IFN | | |
| | | | gamma | | |
| Secondary Th1 rest | 7.5 | 6.8 | HUVEC TNF | 18.2 | 13.4 |
| | | | alpha + IL4 | | |
| Secondary Th2 rest | 11.6 | 9.5 | HUVEC IL-11 | 13.7 | 9.9 |
| Secondary Tr1 rest | 12.1 | 10.7 | Lung | 25.7 | 21.6 |
| | | | Microvascular EC | | |
| | | | none | | |
| Primary Th1 act | 20.7 | 16.5 | Lung | 26.2 | 18.3 |
| | | | Microvascular EC | | |
| | | | TNF alpha + IL- | | |
| | | | 1beta | | |
| Primary Th2 act | 20.2 | 19.3 | Microvascular | 27.5 | 21.3 |
| | | | Dermal EC none | | |
| Primary Tr1 act | 23.3 | 27.7 | Microsvasular | 20.7 | 19.9 |
| | | | Dermal EC | | |
| | | | TNF alpha + IL- | | |
| | | | 1beta | | |
| Primary Th1 rest | 51.1 | 51.4 | Bronchial | 13.0 | 16.3 |
| | | | epithelium | | |
| | | | TNF alpha + | | |
| | | | IL1beta | | |
| Primary Th2 rest | 26.2 | 29.5 | Small airway | 8.1 | 8.5 |
| | | | epithelium none | | |
| Primary Tr1 rest | 23.7 | 26.1 | Small airway | 50.3 | 39.8 |
| | | | epithelium | | |
| | | | TNF alpha + IL- | | |
| | | | 1beta | | |
| CD45RA CD4 | 14.6 | 11.0 | Coronery artery | 20.2 | 18.9 |
| lymphocyte act | | | SMC rest | | |
| CD45RO CD4 | 25.2 | 22.4 | Coronery artery | 12.0 | 9.8 |
| lymphocyte act | | | SMC TNF alpha + | | |
| | | | IL-1beta | | |
| CD8 lymphocyte | 20.4 | 15.8 | Astrocytes rest | 10.4 | 11.1 |
| act | | | | | |
| Secondary CD8 | 16.5 | 19.9 | Astrocytes | 11.7 | 9.8 |
| lymphocyte rest | | | TNF alpha + IL- | | |
| | | | 1beta | | |
| Secondary CD8 | 13.2 | 9.3 | KU-812 | 47.6 | 38.2 |
| lymphocyte act | | | (Basophil) rest | | |
| CD4 lymphocyte | 17.1 | 11.6 | KU-812 | 94.0 | 92.0 |
| none | | | (Basophil) | | |
| | | | PMA/ionomycin | | |
| 2ry | 18.3 | 16.6 | CCD1106 | 19.9 | 13.2 |
| Th1/Th2/Tr1_anti- | | | (Keratinocytes) | | |
| CD95 CH11 | | | none | | |
| LAK cells rest | 25.5 | 16.0 | CCD1106 | 6.0 | 4.8 |
| | | | (Keratinocytes) | | |
| | | | TNF alpha + IL- | | |
| | | | 1beta | | |
| LAK cells IL-2 | 27.2 | 22.5 | Liver cirrhosis | 3.1 | 2.7 |
| LAK cells IL-2 + IL- | 27.2 | 19.3 | Lupus kidney | 2.1 | 1.7 |
| 12 | | | | | |
| LAK cells IL-2 + | 36.3 | 34.4 | NCI-H292 none | 30.1 | 18.9 |
| IFN gamma | | | | | |
| LAK cells IL-2 + | 35.1 | 29.7 | NCI-H292 IL-4 | 33.9 | 34.6 |
| IL-18 | | | | | |
| LAK cells | 12.4 | 11.0 | NCI-H292 IL-9 | 40.1 | 29.1 |
| PMA/ionomycin | | | | | |
| NK Cells IL-2 rest | 20.0 | 15.0 | NCI-H292 IL-13 | 16.2 | 14.2 |
| Two Way MLR 3 | 24.0 | 16.7 | NCI-H292 IFN | 16.6 | 18.4 |
| day | | | gamma | | |
| Two Way MLR 5 | 12.9 | 10.1 | HPAEC none | 13.6 | 13.5 |
| day | | | | | |
| Two Way MLR 7 | 11.4 | 9.5 | HPAEC TNF | 25.3 | 25.3 |
| day | | | alpha + IL-1beta | | |
| PBMC rest | 13.7 | 10.5 | Lung fibroblast | 11.4 | 14.2 |
| | | | none | | |
| PBMC PWM | 69.3 | 66.4 | Lung fibroblast | 6.1 | 7.2 |
| | | | TNF alpha + IL- | | |
| | | | 1beta | | |
| PBMC PHA-L | 22.8 | 17.7 | Lung fibroblast | 28.5 | 29.1 |
| | | | IL-4 | | |
| Ramos (B cell) | 24.1 | 19.3 | Lung fibroblast | 23.0 | 23.3 |
| none | | | IL-9 | | |
| Ramos (B cell) | 100.0 | 100.0 | Lung fibroblast | 20.6 | 18.9 |
| ionomycin | | | IL-13 | | |
| B lymphocytes | 71.7 | 74.2 | Lung fibroblast | 39.0 | 32.5 |
| PWM | | | IFN gamma | | |
| B lymphocytes | 29.1 | 28.7 | Dermal fibroblast | 33.9 | 31.0 |
| CD40L and IL-4 | | | CCD1070 rest | | |
| EOL-1 dbcAMP | 12.1 | 10.5 | Dermal fibroblast | 76.8 | 62.0 |
| | | | CCD1070 TNF | | |
| | | | alpha | | |
| EOL-1 dbcAMP | 14.5 | 10.9 | Dermal fibroblast | 20.3 | 13.9 |
| PMA/ionomycin | | | CCD1070 IL- | | |
| | | | 1beta | | |
| Dendritic cells | 13.2 | 14.8 | Dermal fibroblast | 14.2 | 9.5 |
| none | | | IFN gamma | | |
| Dendritic cells LPS | 11.7 | 8.3 | Dermal fibroblast | 26.4 | 20.4 |
| | | | IL-4 | | |
| Dendritic cells anti- | 17.7 | 12.7 | IBD Colitis 2 | 2.6 | 2.2 |
| CD40 | | | | | |
| Monocytes rest | 16.7 | 17.6 | IBD Crohn's | 2.0 | 1.9 |
| Monocytes LPS | 6.4 | 5.0 | Colon | 11.9 | 10.5 |
| Macrophages rest | 23.5 | 22.8 | Lung | 13.3 | 11.2 |
| Macrophages LPS | 9.9 | 7.1 | Thymus | 14.4 | 12.9 |
| HUVEC none | 20.6 | 17.9 | Kidney | 27.5 | 19.6 |
| HUVEC starved | 43.5 | 38.4 | | | |

CNS_neurodegeneration_v1.0 Summary: Ag3023/Ag3373 This panel does not show differential expression of the NOV77 gene in Alzheimer's disease. However, this expression profile confirms the presence of this gene in the brain. Please see Panel 1.3D for discussion of utility of this gene in the central nervous system.

General_screening_panel_v1.4 Summary: Ag3373 Highest expression of the NOV77 gene is seen in a prostate cancer cell line (CT=27). Overall, this gene is expressed at moderate levels in the cancer cell lines in this panel. A higher level of expression is observed in clusters of cell lines derived from prostate, brain, melanoma, colon, lung, breast and ovarian cancer when compared to expression in normal prostate, brain, colon, lung, breast and ovary. Thus, this gene could potentially be used as a diagnostic marker of cancer in these tissues. Furthermore, inhibition of the activity of this gene product using small molecule drugs may be effective in the treatment of cancer in these tissues.

Among tissues with metabolic function, this gene product has moderate levels of expression in adipose, heart, skeletal muscle, adrenal, pituitary, thyroid and pancreas. Thus, this gene product may be a small molecule target for the treatment of endocrine and metabolic diseases, including obesity and Types 1 and 2 diabetes.

In addition, this gene appears to be differentially expressed in fetal (CT value=29) vs adult liver (CT value=33) and may be useful for differentiation between the two sources of this tissue.

This gene is also expressed at moderate levels in all central nervous system samples present on this panel. Please see Panel 1.3D for discussion of utility of this gene in the central nervous system.

Panel 1.3D Summary: Ag3023 The NOV77 gene is ubiquitously expressed among the samples on this panel, with highest expression in an ovarian cancer cell line (CT=28.8). Overall, the expression of this gene shows good agreement with panel 1.4. A higher level of expression is observed in prostate, brain, melanoma, colon, lung, pancreatic, breast and ovarian cancer cell lines than the normal prostate, brain, colon, lung, pancreas, breast and ovary. Thus, expression of this gene could be used as a diagnostic marker of cancer in these tissues. Furthermore, inhibition of the activity of this gene product using small molecule drugs may be effective in the treatment of cancer in these tissues.

Among tissues with metabolic function, expression of this gene is widespread, as in the previous panel. Please see Panel 1.4 for discussion of utility of this gene in metabolic disease.

This gene represents a dual specificity phosphatase that is also expressed at low to moderate levels across the CNS. Dual-specificity phosphatases comprise a family of MAP kinase regulating enzymes, members of which are upregulated in brains subjected to insults such as ischemia and seizure activity. MAP kinases are kown to regulate neurotrophic and neurotoxic pathways. Consequently, agents that modulate the activity of this gene may have utility in attenuating the apoptotic and neurodegenerative processes following brain insults.

References:

Wiessner C. The dual specificity phosphatase PAC-1 is transcriptionally induced in the rat brain following transient forebrain ischemia. Brain Res Mol Brain Res February 1995;28(2):353-6

PAC-1 mRNA has previously been found only in activated T-cells in vitro and in vivo. The gene encodes a dual specificity protein phosphatase that regulates MAP kinase activity. Here, I describe that PAC-1 mRNA is induced also in neurons in the rat brain following 30 min of forebrain ischemia. At 6, 12 and 24 h after ischemia, PAC-1 mRNA was found most prominently in hippocampal cells which are resistant to 30 min of forebrain ischemia, but not in the selectively vulnerable CA1 sector. At later time points and in control animals no PAC-1 mRNA could be detected in any brain region. The protein-tyrosine/threonine phosphatase PAC-1, therefore, may be involved in adaptational responses of hippocampal cells resistant to ischemic injury.

Boschert U, Muda M, Camps M, Dickinson R, Arkinstall S. Induction of the dual specificity phosphatase PAC1 in rat brain following seizure activity. Neuroreport Sep. 29, 1997;28(14):3077-80

Recurrent seizure activity leads to delayed neuronal death as well as to inflammatory responses involving microglia in hippocampal subfields CA1, CA3 and CA4. Since mitogen activated protein (MAP) kinases control neuronal apoptosis and trigger generation of inflammatory cytokines, their activation state could determine seizure-related brain damage. PAC1 is a dual specificity protein phosphatase inactivating MAP kinases which we have found to be undetectable in normal brain. Despite this, kainic acid-induced seizure activity lead to rapid (approximately 3 h) but transient appearance of PAC1 mRNA in granule cells of the dentate gyrus as well as in pyramidal CA1 neurons. This pattern changed with time and after 2-3 days PAC1 was induced in dying CA1 and CA3 neurons. At this time PAC1 mRNA was also expressed in white matter microglia as well as in microglia invading the damaged hippocampus. PAC1 may play an important role controlling MAP kinase involvement in both neuronal death and neuro-inflammation following excitotoxic damage.

Panel 4D Summary: Ag3023/Ag3373 The NOV77 gene is expressed at high to moderate levels in a wide range of cell types and tissues of significance in the immune response in health and disease. Highest expression of this gene is seen in ionomycin treated Ramos B cells (CT=26.83). Therefore, targeting of this gene product with a small molecule drug or antibody therapeutic may modulate the functions of cells of the immune system as well as resident tissue cells and lead to improvement of the symptoms of patients suffering from autoimmune and inflammatory diseases such as asthma, allergies, inflammatory bowel disease, lupus erythematosus, and arthritis, including osteoarthritis and rheumatoid arthritis.

NOV78

Expression of gene NOV78 was assessed using the primer-probe set Ag3025, described in Table BSA. Results of the RTQ-PCR runs are shown in Tables BSB, BSC and BSD.

TABLE BSA
Probe Name Ag3025

| | | | Start | |
|---|---|---|---|---|
| Primers | Sequences | Length | Position | SEQ ID NO: |
| Forward | 5′-gctgctgcaattgtaataggtt-3′ | 22 | 596 | 1309 |
| Probe | TET-5′-tcctgatgaattctgaacaaaacctca-3′-TAMRA | 26 | 618 | 1310 |
| Reverse | 5′-catatggaaggtcttgcattt-3′ | 22 | 669 | 1311 |

TABLE BSB
CNS_neurodegeneration_v1.0

| | Rel. Exp. (%) Ag3025, | | Rel. Exp. (%) Ag3025, |
|---|---|---|---|
| Tissue Name | Run 209821733 | Tissue Name | Run 209821733 |
| AD 1 Hippo | 21.3 | Control (Path) 3 | 4.8 |
| | | Temporal Ctx | |
| AD 2 Hippo | 29.1 | Control (Path) 4 | 50.7 |
| | | Temporal Ctx | |
| AD 3 Hippo | 15.9 | AD 1 Occipital Ctx | 22.7 |
| AD 4 Hippo | 11.4 | AD 2 Occipital Ctx | 0.0 |
| | | (Missing) | |
| AD 5 Hippo | 66.4 | AD 3 Occipital Ctx | 9.0 |
| AD 6 Hippo | 26.6 | AD 4 Occipital Ctx | 13.7 |
| Control 2 Hippo | 32.5 | AD 5 Occipital Ctx | 54.7 |
| Control 4 Hippo | 7.2 | AD 6 Occipital Ctx | 8.1 |
| Control (Path) 3 | 4.8 | Control 1 Occipital | 2.3 |
| Hippo | | Ctx | |
| AD 1 Temporal Ctx | 19.9 | Control 2 Occipital | 40.1 |
| | | Ctx | |
| AD 2 Temporal Ctx | 29.9 | Control 3 Occipital | 13.8 |
| | | Ctx | |
| AD 3 Temporal Ctx | 9.3 | Control 4 Occipital | 5.6 |
| | | Ctx | |
| AD 4 Temporal Ctx | 25.2 | Control (Path) 1 | 84.7 |
| | | Occipital Ctx | |
| AD 5 Inf Temporal | 79.6 | Control (Path) 2 | 10.0 |
| Ctx | | Occipital Ctx | |
| AD 5 Sup Temporal | 56.6 | Control (Path) 3 | 0.6 |
| Ctx | | Occipital Ctx | |
| AD 6 Inf Temporal | 19.3 | Control (Path) 4 | 12.3 |
| Ctx | | Occipital Ctx | |
| AD 6 Sup Temporal | 24.5 | Control 1 Parietal | 3.8 |
| Ctx | | Ctx | |
| Control 1 Temporal | 9.7 | Control 2 Parietal | 30.1 |
| Ctx | | Ctx | |
| Control 2 Temporal | 43.5 | Control 3 Parietal | 21.6 |
| Ctx | | Ctx | |
| Control 3 Temporal | 15.2 | Control (Path) 1 | 100.0 |
| Ctx | | Parietal Ctx | |
| Control 3 Temporal | 7.4 | Control (Path) 2 | 34.9 |
| Ctx | | Parietal Ctx | |
| Control (Path) 1 | 75.3 | Control (Path) 3 | 1.5 |
| Temporal Ctx | | Parietal Ctx | |
| Control (Path) 2 | 35.6 | Control (Path) 4 | 52.1 |
| Temporal Ctx | | Parietal Ctx | |

TABLE BSC
Panel 1.3D

| | Rel. Exp. (%) Ag3025, | | Rel. Exp. (%) Ag3025, |
|---|---|---|---|
| Tissue Name | Run 167968622 | Tissue Name | Run 167968622 |
| Liver adenocarcinoma | 3.6 | Kidney (fetal) | 16.3 |
| Pancreas | 6.8 | Renal ca. 786-0 | 6.2 |
| Pancreatic ca. CAPAN 2 | 2.7 | Renal ca. A498 | 10.9 |
| Adrenal gland | 2.3 | Renal ca. RXF 393 | 6.7 |
| Thyroid | 7.2 | Renal ca. ACHN | 3.3 |
| Salivary gland | 3.2 | Renal ca. UO-31 | 5.3 |
| Pituitary gland | 16.3 | Renal ca. TK-10 | 7.2 |
| Brain (fetal) | 37.6 | Liver | 3.8 |
| Brain (whole) | 33.2 | Liver (fetal) | 3.4 |
| Brain (amygdala) | 29.7 | Liver ca. | 2.5 |
| | | (hepatoblast) HepG2 | |
| Brain (cerebellum) | 9.7 | Lung | 4.6 |
| Brain (hippocampus) | 21.9 | Lung (fetal) | 0.0 |
| Brain (substantia nigra) | 12.2 | Lung ca. (small cell) | 3.8 |
| | | LX-1 | |
| Brain (thalamus) | 10.4 | Lung ca. (small cell) | 2.5 |
| | | NCI-H69 | |
| Cerebral Cortex | 16.3 | Lung ca. (s.cell var.) | 100.0 |
| | | SHP-77 | |
| Spinal cord | 10.7 | Lung ca. (large | 0.8 |
| | | cell) NCI-H460 | |
| glio/astro U87-MG | 6.0 | Lung ca. (non-sm. | 5.8 |
| | | cell) A549 | |
| glio/astro U-118-MG | 8.8 | Lung ca. (non-s.cell) | 15.4 |
| | | NCI-H23 | |
| astro cytoma SW1783 | 15.3 | Lung ca. (non-s.cell) | 7.7 |
| | | HOP-62 | |
| neuro*; met SK-N-AS | 6.4 | Lung ca. (non-s.cl) | 13.4 |
| | | NCI-H522 | |
| astrocytoma SF-539 | 5.3 | Lung ca. (squam.) | 1.7 |
| | | SW 900 | |
| astrocytoma SNB-75 | 7.1 | Lung ca. (squam.) | 3.7 |
| | | NCI-H596 | |
| glioma SNB-19 | 6.3 | Mammary gland | 4.1 |
| glioma U251 | 9.9 | Breast ca.* (pl.ef) | 8.7 |
| | | MCF-7 | |
| glioma SF-295 | 10.4 | Breast ca.* (pl.ef) | 1.7 |
| | | MDA-MB-231 | |
| Heart (fetal) | 3.3 | Breast ca.* (pl.ef) | 14.1 |
| | | T47D | |
| Heart | 20.3 | Breast ca. BT-549 | 3.6 |
| Skeletal muscle (fetal) | 0.5 | Breast ca. MDA-N | 15.8 |
| Skeletal muscle | 15.5 | Ovary | 9.0 |
| Bone marrow | 2.8 | Ovarian ca. OVCAR-3 | 3.5 |
| Thymus | 11.9 | Ovarian ca. OVCAR-4 | 1.0 |
| Spleen | 5.6 | Ovarian ca. OVCAR-5 | 19.2 |
| Lymph node | 3.1 | Ovarian ca. OVCAR-8 | 5.1 |
| Colorectal | 4.9 | Ovarian ca. IGROV-1 | 0.6 |
| Stomach | 11.9 | Ovarian ca.* (ascites) | 52.1 |
| | | SK-OV-3 | |
| Small intestine | 6.2 | Uterus | 7.3 |
| Colon ca. SW480 | 0.3 | Placenta | 3.1 |
| Colon ca.* | 17.0 | Prostate | 1.4 |
| SW620 (SW480 met) | | | |
| Colon ca. HT29 | 2.8 | Prostate ca.* (bone | 11.6 |
| | | met)PC-3 | |
| Colon ca. HCT-116 | 5.1 | Testis | 5.4 |
| Colon ca. CaCo-2 | 15.2 | Melanoma | 2.2 |
| | | Hs688(A).T | |
| Colon ca. | 1.7 | Melanoma* (met) | 2.2 |
| tissue (ODO3866) | | Hs688(B).T | |
| Colon ca. HCC-2998 | 9.0 | Melanoma UACC-62 | 12.2 |
| Gastric ca.* (liver met) | 3.2 | Melanoma M14 | 3.6 |
| NCI-N87 | | | |
| Bladder | 3.1 | Melanoma LOX | 3.5 |
| | | IMVI | |
| Trachea | 5.2 | Melanoma* (met) | 11.8 |
| | | SK-MEL-5 | |
| Kidney | 17.4 | Adipose | 7.9 |

TABLE BSD
Panel 4D

| | Rel. Exp. (%) | | Rel. Exp. (%) |
|---|---|---|---|
| | Ag3025, Run | | Ag3025, Run |
| Tissue Name | 164528140 | Tissue Name | 164528140 |
| Secondary Th1 act | 2.6 | HUVEC IL-1beta | 12.5 |
| Secondary Th2 act | 3.3 | HUVEC IFN gamma | 9.5 |
| Secondary Tr1 act | 4.8 | HUVEC TNF alpha + IFN | 5.1 |
| | | gamma | |
| Secondary Th1 rest | 1.2 | HUVEC TNF alpha + IL4 | 5.1 |
| Secondary Th2 rest | 2.7 | HUVEC IL-11 | 3.4 |
| Secondary Tr1 rest | 1.4 | Lung Microvascular EC | 10.7 |
| | | none | |
| Primary Th1 act | 6.2 | Lung Microvascular EC | 13.5 |
| | | TNF alpha + IL-1beta | |
| Primary Th2 act | 3.5 | Microvascular Dermal EC | 6.0 |
| | | none | |
| Primary Tr1 act | 2.5 | Microsvasular Dermal EC | 2.6 |
| | | TNF alpha + IL-1beta | |
| Primary Th1 rest | 5.4 | Bronchial epithelium | 6.2 |
| | | TNF alpha + IL1beta | |
| Primary Th2 rest | 6.7 | Small airway epithelium | 0.5 |
| | | none | |
| Primary Tr1 rest | 1.4 | Small airway epithelium | 8.0 |
| | | TNF alpha + IL-1beta | |
| CD45RA CD4 | 4.4 | Coronery artery SMC rest | 32.5 |
| lymphocyte act | | | |
| CD45RO CD4 | 4.0 | Coronery artery SMC | 6.9 |
| lymphocyte act | | TNF alpha + IL-1beta | |
| CD8 lymphocyte act | 16.8 | Astrocytes rest | 9.2 |
| Secondary CD8 | 5.2 | Astrocytes TNF alpha + | 3.4 |
| lymphocyte rest | | IL-1beta | |
| Secondary CD8 | 1.9 | KU-812 (Basophil) rest | 7.3 |
| lymphocyte act | | | |
| CD4 lymphocyte none | 2.3 | KU-812 (Basophil) | 29.5 |
| | | PMA/ionomycin | |
| 2ry Th1/Th2/Tr1_anti- | 2.6 | CCD1106 (Keratinocytes) | 1.3 |
| CD95 CH11 | | none | |
| LAK cells rest | 5.8 | CCD1106 (Keratinocytes) | 0.0 |
| | | TNF alpha + IL-1beta | |
| LAK cells IL-2 | 21.0 | Liver cirrhosis | 3.7 |
| LAK cells IL-2 + IL-12 | 11.3 | Lupus kidney | 3.7 |
| LAK cells IL-2 + IFN | 12.4 | NCI-H292 none | 12.2 |
| gamma | | | |
| LAK cells IL-2 + IL-18 | 67.4 | NCI-H292 IL-4 | 10.2 |
| LAK cells | 0.6 | NCI-H292 IL-9 | 100.0 |
| PMA/ionomycin | | | |
| NK Cells IL-2 rest | 4.2 | NCI-H292 IL-13 | 5.1 |
| Two Way MLR 3 day | 3.5 | NCI-H292 IFN gamma | 2.7 |
| Two Way MLR 5 day | 1.3 | HPAEC none | 9.3 |
| Two Way MLR 7 day | 4.9 | HPAEC TNF alpha + IL-1 | 2.4 |
| | | beta | |
| PBMC rest | 4.2 | Lung fibroblast none | 5.5 |
| PBMC PWM | 24.7 | Lung fibroblast TNF alpha + | 6.3 |
| | | IL-1beta | |
| PBMC PHA-L | 4.3 | Lung fibroblast IL-4 | 6.8 |
| Ramos (B cell) none | 19.1 | Lung fibroblast IL-9 | 10.4 |
| Ramos (B cell) | 33.4 | Lung fibroblast IL-13 | 4.5 |
| ionomycin | | | |
| B lymphocytes PWM | 19.2 | Lung fibroblast IFN | 15.2 |
| | | gamma | |
| B lymphocytes CD40L | 5.2 | Dermal fibroblast | 17.8 |
| and IL-4 | | CCD1070 rest | |
| EOL-1 dbcAMP | 0.7 | Dermal fibroblast | 36.6 |
| | | CCD1070 TNF alpha | |
| EOL-1 dbcAMP | 0.9 | Dermal fibroblast | 6.5 |
| PMA/ionomycin | | CCD1070 IL-1beta | |
| Dendritic cells none | 3.7 | Dermal fibroblast IFN | 0.6 |
| | | gamma | |
| Dendritic cells LPS | 6.0 | Dermal fibroblast IL-4 | 6.6 |
| Dendritic cells anti- | 3.3 | IBD Colitis 2 | 0.7 |
| CD40 | | | |
| Monocytes rest | 2.4 | IBD Crohn's | 0.8 |
| Monocytes LPS | 0.6 | Colon | 21.5 |
| Macrophages rest | 5.6 | Lung | 18.3 |
| Macrophages LPS | 0.8 | Thymus | 52.1 |
| HUVEC none | 6.6 | Kidney | 19.2 |
| HUVEC starved | 21.9 | | |

CNS_neurodegeneration_v1.0 Summary: Ag3025 This panel does not show differential expression of the NOV78 gene in Alzheimer's disease. However, this expression profile confirms the presence of this gene in the brain. Please see Panel 1.3D for discussion of utility of this gene in the central nervous system.

Panel 1.3D Summary: Ag3025 Highest expression of the NOV78 gene is seen in a lung cancer cell line (CT=30.5). Higher levels of expression are observed in prostate, lung, breast and ovarian cancer cell lines when compared with the normal prostate, lung, breast and ovary. Thus, expression of this gene may be used as a diagnostic marker of cancer in these tissues. Furthermore, inhibition of the activity of this gene product using small molecule drugs may be effective in the treatment of cancer in these tissues.

Among tissues with metabolic function, this gene product using small molecule drugs pancreas, thyroid, pituitary, heart, and adipose. Therefore, this gene product may be a small molecule target for the treatment of metabolic and endocrine diseases, including obesity and Types 1 and 2 diabetes.

This gene represents a dual specificity phosphatase that is also expressed at low to moderate levels across the CNS. Dual-specificity phosphatases comprise a family of MAP kinase regulating enzymes that are upregulated in brains subjected to insults such as ischemia and seizure activity. MAP kinases are known to regulate neurotrophic and neurotoxic pathways. Consequently, agents that modulate the activity of this gene may have utility in attenuating the apoptotic and neurodegenerative processes following brain insults.

References:

Wiessner C. The dual specificity phosphatase PAC-1 is transcriptionally induced in the rat brain following transient forebrain ischemia. Brain Res Mol Brain Res February 1995;28(2):353-6

PAC-1 mRNA has previously been found only in activated T-cells in vitro and in vivo. The gene encodes a dual specificity protein phosphatase that regulates MAP kinase activity. Here, I describe that PAC-1 mRNA is induced also in neurons in the rat brain following 30 min of forebrain ischemia. At 6, 12 and 24 h after ischemia, PAC-1 mRNA was found most prominently in hippocampal cells which are resistant to 30 min of forebrain ischemia, but not in the selectively vulnerable CA1 sector. At later time points and in control animals no PAC-1 mRNA could be detected in any brain region. The protein-tyrosine/threonine phosphatase PAC-1, therefore, may be involved in adaptational responses of hippocampal cells resistant to ischemic injury.

Boschert U, Muda M, Camps M, Dickinson R, Arkinstall S. Induction of the dual specificity phosphatase PAC1 in rat brain following seizure activity. Neuroreport Sep. 29, 1997;8(14):3077-80

Recurrent seizure activity leads to delayed death as well as to inflammatory responses involving microglia in hippocampal subfields CA1, CA3 and CA4. Since mitogen activated protein (MAP) kinases control neuronal apoptosis and trigger generation of inflammatory cytokines, their activation state could determine seizure-related brain damage. PAC1 is a dual specificity protein phosphatase inactivating MAP kinases which we have found to be undetectable in normal brain. Despite this, kainic acid-induced seizure activity lead to rapid (approximately 3 h) but transient appearance of PAC1 mRNA is granule cells of the dentate gyrus as well as in pyramidal CA1 neurons. This pattern changed with time and after 2-3 days PAC1 was induced in dying CA1 and CA3 neurons. At this time PAC1 mRNA was also expressed in white matter microglia as well as in microglia invading the damaged hippocampus. PAC1 may play an important role controlling MAP kinase involvement in both neuronal death and neuro-inflammation following excitotoxic damage.

Panel 4D Summary: Ag3025 The NOV78 gene is expressed at moderate to low levels in a wide range of cell types of significance Highest expression is detected in IL-9 treated NCI-H292 mucoepidermoid cells (CT=31.81) with lower expression levels in non-treated NCI-H292 cells. Expression is also seen in (i) LAK cells stimulated with IL-2, IL-2+IL-12, IL-2+IL-18, and IL-2+IFNgamma (ii) stimulated and non-stimulated Ramos B cells and polkweek mitogen stimulated B lymphocytes, (iii) starved and IL-1 treated HUVECs, (iv) TNF alpha+IL-1 beta treated and non treated lung microvascular endothelial cells and resting coronary artery smooth muscle cells (v) treated Ku-812 basophils (vi) IFN gamma treated lung fibroblasts, and (vii) normal tissues represented by colon, lung, thymus and kidney. Based on this pattern of expression, this gene product may be involved in both disease and homeostatic processes for these and other cell types and tissues. Therefore, modulation of this gene product with a functional therapeutic may lead to the alteration of functions associated with these cell and tissue types and improvement of the symptoms of patients suffering from autoimmune and inflammatory diseases such as COPD, emphysema, asthma, allergies, inflammatory bowel disease, lupus erythematosus, psoriasis, rheumatoid arthritis, and osteoarthritis.

NOV79: Dual Specificity Phosphatase

Expression of gene NOV79 was assessed using the primer-probe set Ag3039, described in Table BTA. Results of the RTQ-PCR runs are shown in Tables BTB, BTC and BTD.

TABLE BTA
Probe Name Ag3039

| | | | | |
|---|---|---|---|---|
| | | | Start | |
| Primers | Sequences | Length | Position | SEQ ID NO: |

| | | | | |
|---|---|---|---|---|
| Forward | 5′-gccgaaataagatcacacacat-3′ | 22 | 320 | 1312 |
| Probe | TET-5′-tctatccatgagtcaccccagcctct-3′-TAMRA | 26 | 346 | 1313 |
| Reverse | 5′-atgcgaaggtaggtgatatcct-3′ | 22 | 377 | 1314 |

TABLE BTB
CNS_neurodegeneration_v1.0

| | Rel. Exp. (%) Ag3039, | | Rel. Exp. (%) Ag3039, |
|---|---|---|---|
| Tissue Name | Run 211012103 | Tissue Name | Run 211012103 |
| AD 1 Hippo | 18.4 | Control (Path) 3 | 8.2 |
| | | Temporal Ctx | |
| AD 2 Hippo | 48.0 | Control (Path) 4 | 36.3 |
| | | Temporal Ctx | |
| AD 3 Hippo | 9.8 | AD 1 Occipital Ctx | 9.5 |
| AD 4 Hippo | 13.6 | AD 2 Occipital Ctx | 0.0 |
| | | (Missing) | |
| AD 5 hippo | 70.2 | AD 3 Occipital Ctx | 6.3 |
| AD 6 Hippo | 69.3 | AD 4 Occipital Ctx | 20.9 |
| Control 2 Hippo | 25.5 | AD 5 Occipital Ctx | 18.3 |
| Control 4 Hippo | 24.0 | AD 6 Occipital Ctx | 43.2 |
| Control (Path) 3 | 7.6 | Control 1 Occipital | 6.0 |
| Hippo | | Ctx | |
| AD 1 Temporal Ctx | 24.3 | Control 2 Occipital | 57.0 |
| | | Ctx | |
| AD 2 Temporal Ctx | 36.9 | Control 3 Occipital | 18.7 |
| | | Ctx | |
| AD 3 Temporal Ctx | 4.7 | Control 4 Occipital | 13.9 |
| | | Ctx | |
| AD 4 Temporal Ctx | 24.5 | Control (Path) 1 | 74.2 |
| | | Occipital Ctx | |
| AD 5 Inf Temporal | 100.0 | Control (Path) 2 | 14.8 |
| Ctx | | Occipital Ctx | |
| AD 5 SupTemporal | 62.9 | Control (Path) 3 | 4.3 |
| Ctx | | Occipital Ctx | |
| AD 6 Inf Temporal | 58.2 | Control (Path) 4 | 25.2 |
| Ctx | | Occipital Ctx | |
| AD 6 Sup Temporal | 49.3 | Control 1 Parietal | 15.9 |
| Ctx | | Ctx | |
| Control 1 Temporal | 11.6 | Control 2 Parietal | 58.2 |
| Ctx | | Ctx | |
| Control 2 Temporal | 34.4 | Control 3 Parietal | 32.1 |
| Ctx | | Ctx | |
| Control 3 Temporal | 20.0 | Control (Path) 1 | 66.9 |
| Ctx | | Parietal Ctx | |
| Control 4 Temporal | 20.7 | Control (Path) 2 | 39.0 |
| Ctx | | Parietal Ctx | |
| Control (Path) 1 | 44.4 | Control (Path) 3 | 4.6 |
| Temporal Ctx | | Parietal Ctx | |
| Control (Path) 2 | 30.4 | Control (Path) 4 | 35.8 |
| Temporal Ctx | | Parietal Ctx | |

TABLE BTC
Panel 1.3D

| | Rel. Exp. (%) Ag3039, | | Rel. Exp. (%) Ag3039, |
|---|---|---|---|
| Tissue Name | Run 167961816 | Tissue Name | Run 167961816 |
| Liver adenocarcinoma | 1.7 | Kidney (fetal) | 38.2 |
| Pancreas | 2.0 | Renal ca. 786-0 | 0.0 |
| Pancreatic ca. CAPAN 2 | 0.0 | Renal ca. A498 | 0.4 |
| Adrenal gland | 1.2 | Renal ca. RXF 393 | 0.1 |
| Thyroid | 6.0 | Renal ca. ACHN | 1.5 |
| Salivary gland | 0.8 | Renal ca. UO-31 | 0.0 |
| Pituitary gland | 3.0 | Renal ca. TK-10 | 0.0 |
| Brain (fetal) | 7.4 | Liver | 0.3 |
| Brain (whole) | 7.7 | Liver (fetal) | 0.7 |
| Brain (amygdala) | 6.0 | Liver ca. | 0.1 |
| | | (hepatoblast) HepG2 | |
| Brain (cerebellum) | 5.9 | Lung | 1.3 |
| Brain (hippocampus) | 3.9 | Lung (fetal) | 3.0 |
| Brain (substantia nigra) | 16.0 | Lung ca. (small cell) | 0.0 |
| | | LX-1 | |
| Brain (thalamus) | 3.8 | Lung ca. (small cell) | 1.5 |
| | | NCI-H69 | |
| Cerebral Cortex | 10.8 | Lung ca. (s.cell var.) | 3.7 |
| | | SHP-77 | |
| Spinal cord | 15.2 | Lung ca. (large | 0.1 |
| | | cell) NCI-H460 | |
| glio/astro U87-MG | 0.2 | Lung ca. (non-sm. | 1.6 |
| | | cell) A549 | |
| glio/astro U-118-MG | 0.1 | Lung ca. (non-s.cell) | 0.4 |
| | | NCI-H23 | |
| astrocytoma SW1783 | 0.6 | Lung ca. (non-s.cell) | 0.0 |
| | | HOP-62 | |
| neuro*; met SK-N-AS | 0.3 | Lung ca. (non-s.cl) | 1.4 |
| | | NCI-H522 | |
| astrocytoma SF-539 | 1.5 | Lung ca. (squam.) | 0.7 |
| | | SW 900 | |
| astrocytoma SNB-75 | 1.2 | Lung ca. (squam.) | 5.7 |
| | | NCI-H596 | |
| glioma SNB-19 | 2.1 | Mammary gland | 2.4 |
| glioma U251 | 0.3 | Breast ca.* (pl.ef) | 0.7 |
| | | MCF-7 | |
| glioma SF-295 | 2.3 | Breast ca.* (pl.ef) | 0.1 |
| | | MDA-MB-231 | |
| Heart (fetal) | 14.5 | Breast ca.* (pl.ef) | 13.5 |
| | | T47D | |
| Heart | 3.4 | Breast ca. BT-549 | 1.2 |
| Skeletal muscle (fetal) | 5.1 | Breast ca. MDA-N | 6.4 |
| Skeletal muscle | 0.0 | Ovary | 3.9 |
| Bone marrow | 0.4 | Ovarian ca. OVCAR-3 | 0.3 |
| Thymus | 0.2 | Ovarian ca. OVCAR-4 | 13.8 |
| Spleen | 3.1 | Ovarian ca. OVCAR-5 | 1.5 |
| Lymph node | 0.9 | Ovarian ca. OVCAR-8 | 0.0 |
| Colorectal | 0.4 | Ovarian ca. IGROV-1 | 0.0 |
| Stomach | 0.6 | Ovarian ca.* (ascites) | 0.4 |
| | | SK-OV-3 | |
| Small intestine | 1.0 | Uterus | 2.2 |
| Colon ca. SW480 | 6.7 | Placenta | 0.0 |
| Colon ca.* | 0.0 | Prostate | 0.0 |
| SW620 (SW480 met) | | | |
| Colon ca. HT29 | 0.0 | Prostate ca.* (bone | 0.0 |
| | | met)PC-3 | |
| Colon ca. HCT-116 | 0.0 | Testis | 100.0 |
| Colon ca. CaCo-2 | 12.2 | Melanoma | 0.1 |
| | | Hs688(A).T | |
| Colon ca. | 1.2 | Melanoma* (met) | 0.0 |
| tissue (ODO3866) | | Hs688(B).T | |
| Colon ca. HCC-2998 | 3.0 | Melanoma UACC-62 | 5.2 |
| Gastric ca.* (liver met) | 1.2 | Melanoma M14 | 0.8 |
| NCI-N87 | | | |
| Bladder | 1.8 | Melanoma LOX | 0.0 |
| | | IMVI | |
| Trachea | 0.6 | Melanoma* (met) | 2.0 |
| | | SK-MEL-5 | |
| Kidney | 41.8 | Adipose | 0.6 |

TABLE BTD
Panel 4D

| | Rel. Exp. (%) | | Rel. Exp. (%) |
|---|---|---|---|
| | Ag3039, Run | | Ag3039, Run |
| Tissue Name | 162427949 | Tissue Name | 162427949 |
| Secondary Th1 act | 0.0 | HUVEC IL-1beta | 0.0 |
| Secondary Th2 act | 0.0 | HUVEC IFN gamma | 0.3 |
| Secondary Tr1 act | 0.0 | HUVEC TNF alpha + IFN | 0.0 |
| | | gamma | |
| Secondary Th1 rest | 0.0 | HUVEC TNF alpha + IL4 | 0.0 |
| Secondary Th2 rest | 0.0 | HUVEC IL-11 | 0.3 |
| Secondary Tr1 rest | 0.0 | Lung Microvascular EC | 1.3 |
| | | none | |
| Primary Th1 act | 0.0 | Lung Microvascular EC | 0.0 |
| | | TNF alpha + IL-1beta | |
| Primary Th2 act | 0.0 | Microvascular Dermal EC | 0.3 |
| | | none | |
| Primary Tr1 act | 0.0 | Microsvascular Dermal EC | 0.4 |
| | | TNF alpha + IL-1beta | |
| Primary Th1 rest | 0.0 | Bronchial epithelium | 1.4 |
| | | TNF alpha + IL1beta | |
| Primary Th2 rest | 0.0 | Small airway epithelium | 1.1 |
| | | none | |
| Primary Tr1 rest | 0.0 | Small airway epithelium | 0.3 |
| | | TNF alpha + IL-1beta | |
| CD45RA CD4 | 0.0 | Coronery artery SMC rest | 0.0 |
| lymphocyte act | | | |
| CD45RO CD4 | 0.0 | Coronery artery SMC | 0.0 |
| lymphocyte act | | TNF alpha + IL-1beta | |
| CD8 lymphocyte act | 0.0 | Astrocytes rest | 4.0 |
| Secondary CD8 | 0.0 | Astrocytes TNF alpha + | 2.3 |
| lymphocyte rest | | IL-1beta | |
| Secondary CD8 | 0.0 | KU-812 (Basophil) rest | 0.0 |
| lymphocyte act | | | |
| CD4 lymphocyte none | 0.0 | KU-812 (Basophil) | 0.0 |
| | | PMA/ionomycin | |
| 2ry Th1/Th2/Tr1_anti- | 0.0 | CCD1106 (Keratinocytes) | 0.4 |
| CD95 CH11 | | none | |
| LAK cells rest | 0.1 | CCD1106 (Keratinocytes) | 0.4 |
| | | TNF alpha + IL-1beta | |
| LAK cells IL-2 | 0.4 | Liver cirrhosis | 0.7 |
| LAK cells IL-2 + IL-12 | 0.0 | Lupus kidney | 5.2 |
| LAK cells IL-2 + IFN | 0.4 | NCI-H292 none | 3.5 |
| gamma | | | |
| LAK cells IL-2 + IL-18 | 0.8 | NCI-H292 IL-4 | 0.9 |
| LAK cells | 0.0 | NCI-H292 IL-9 | 1.8 |
| PMA/ionomycin | | | |
| NK Cells IL-2 rest | 0.0 | NCI-H292 IL-13 | 1.2 |
| Two Way MLR 3 day | 0.0 | NCI-H292 IFN gamma | 3.6 |
| Two Way MLR 5 day | 0.0 | HPAEC none | 0.0 |
| Two Way MLR 7 day | 0.0 | HPAEC TNF alpha + IL-1 | 0.0 |
| | | beta | |
| PBMC rest | 0.3 | Lung fibroblast none | 0.0 |
| PBMC PWM | 2.5 | Lung fibroblast TNF | 0.0 |
| | | alpha + IL-1beta | |
| PBMC PHA-L | 1.6 | Lung fibroblast IL-4 | 0.0 |
| Ramos (B cell) none | 0.0 | Lung fibroblast IL-9 | 0.0 |
| Ramos (B cell) | 0.0 | Lung fibroblast IL-13 | 0.5 |
| ionomycin | | | |
| B lymphocytes PWM | 5.5 | Lung fibroblast IFN | 0.0 |
| | | gamma | |
| B lymphocytes CD40L | 1.3 | Dermal fibroblast | 0.0 |
| and IL-4 | | CCD1070 rest | |
| EOL-1 dbcAMP | 0.6 | Dermal fibroblast | 0.0 |
| | | CCD1070 TNF alpha | |
| EOL-1 dbcAMP | 0.0 | Dermal fibroblast | 0.0 |
| PMA/ionomycin | | CCD1070 IL-1beta | |
| Dendritic cells none | 0.0 | Dermal fibroblast IFN | 0.0 |
| | | gamma | |
| Dendritic cells LPS | 0.4 | Dermal fibroblast IL-4 | 0.0 |
| Dendritic cells anti- | 0.0 | IBD Colitis 2 | 0.0 |
| CD40 | | | |
| Monocytes rest | 0.0 | IBD Crohn's | 1.1 |
| Monocytes LPS | 0.4 | Colon | 2.5 |
| Macrophages rest | 1.0 | Lung | 5.1 |
| Macrophages LPS | 0.4 | Thymus | 100.0 |
| HUVEC none | 0.6 | Kidney | 3.1 |
| HUVEC starved | 0.0 | | |

CNS_neurodegeneration_v1.0 Summary: Ag3039 No differential expression of the NOV79 gene is detected between the postmortem brains of Alzheimer's diseased patients and those of non-demented controls. However, this panel confirms the expression of this gene in the CNS. Please see panel 1.3D for a discussion of utility of this gene in the central nervous system.

Panel 1.3D Summary: Ag3039 Highest expression of the NOV79 gene is seen in the testis (CT=29). In addition, expression of this gene is extremely low in renal and brain cancer cell lines but is expressed in the normal brain and kidney tissues in this sample. Therefore, this gene may be used as a diagnostic marker for brain and kidney cancer and prostate tissue. Further, therapeutic modulation of the expression or function of this gene may be effective in the treatment of brain and renal cancers.

In addition, this gene is expressed at low levels in metabolic tissues including pancreas, adrenal, thyroid, pituitary, adult and fetal heart, and adipose. This novel protein phosphatase may be a small molecule target for the treatment of metabolic and endocrine disease, including obesity and Types 1 and 2 diabetes. This gene is also differentially expressed in fetal (CT values=32-33) vs adult skeletal muscle (CT values=35-40) and may be useful for the differentiation of adult and fetal skeletal muscle.

This gene represents a dual specificity phosphatase that is also expressed at low to moderate levels across the CNS. Dual-specificity phosphatases comprise a family of MAP kinase regulating enzymes that are upregulated in brains subjected to insults such as ischemia and seizure activity. MAP kinases are known to regulate neurotrophic and neurotoxic pathways. Consequently, agents that modulate the activity of this gene may have utility in attenuating the apoptotic and neurodegenerative processes following brain insults.

References:

Wiessner C. The dual specificity phosphatase PAC-1 is transcriptionally induced in the rat brain following transient forebrain ischemia. Brain Res Mol Brain Res February 1995;28(2):353-6

PAC-1 mRNA has previously been found only in activated T-cells in vitro and in vivo. The gene encodes a dual specificity protein phosphatase that regulates MAP kinase activity. Here, I describe that PAC-1 mRNA is induced also in neurons in the rat brain following 30 min of forebrain ischemia. At 6, 12 and 24 h after ischemia, PAC-1 mRNA was found most prominently in hippocampal cells which are resistant to 30 min of forebrain ischemia, but not in the selectively vulnerable CA1 sector. At later time points and in control animals no PAC-1 mRNA could be detected in any brain region. The protein-tyrosine/threonine phosphatase PAC-1, therefore, may be involved in adaptational responses of hippocampal cells resistant to ischemic injury.

Boschert U, Muda M, Camp M, Dickinson R, Arkinstall S. Induction of the dual specificity phosphatase PAC1 in rat brain following seizure activity. Neuroreport Sep. 29, 1997;8(14):3007-80

Recurrent seizure activity leads to delayed neuronal death as well as to inflammatory responses involving microglia in hippocampal subfields CA1, CA3 and CA4. Since mitogen activated protein (MAP) kinases control neuronal apoptosis and trigger generation of inflammatory cytokines, their activation state could determine seizure-related brain damage. PAC1 is a dual specificity protein phospatase inactivating MAP kinases which we have found to be undetectable in normal brain. Despite this, kainic acid-induced seizure activity lead to rapid (approximately 3 h) but transient appearance of PAC1 mRNA in granule cells of the dentate gyrus as well as in pyramidal CA1 neurons. This pattern changed with time and after 2-3 days PAC1 was induced in dying CA1 and CA3 neurons. At this time PAC1 mRNA was also expressed in white matter microglia as well as in microglia invading the damaged hippocampus. PAC1 may play an important role controlling MAP kinase involvement in both neuronal death and neuro-inflammation following excitotoxic damage.

Panel 4D Summary: Ag3039 Expression of the NOV79 gene is highest and almost exclusive to the thymus (CTs=29-30). Expression of this gene could be used to distinguish encoded by this gene may play an important role in T cell development. Small molecule therapeutics designed against the protein encoded by this gene could therefore be utilized to modulate immune function (T cell development) and be important for organ transplant, AIDS treatment or post chemotherapy immune reconstitiution.

NOV80

Expression of gene NOV80 was assessed using the primer-probe set Ag3044, described in Table BUA. Results of the RTQ-PCR runs are shown in Tables BUB and BUC.

TABLE BUA
Probe Name Ag3044

| | | | Start | |
|---|---|---|---|---|
| Primers | Sequences | Length | Position | SEQ ID NO: |
| Forward | 5′-tgacgcagaatggaataagct-3′ | 21 | 650 | 1315 |
| Probe | TET-5′-acgtcctctatgccagcaactcctg-3′-TAMRA | 25 | 671 | 1316 |
| Reverse | 5′-gcaagaagtggctctggtagat-3′ | 22 | 712 | 1317 |

Table BUB
Panel 1.3D

| | Rel. Exp. (%) Ag3044, | | Rel. Exp. (%) Ag3044, |
|---|---|---|---|
| Tissue Name | Run 167972762 | Tissue Name | Run 167972762 |
| Liver adenocarcinoma | 0.1 | Kidney (fetal) | 0.5 |
| Pancreas | 0.1 | Renal ca. 786-0 | 0.2 |
| Pancreatic ca. CAPAN 2 | 0.0 | Renal ca. A498 | 0.0 |
| Adrenal gland | 0.0 | Renal ca. RXF 393 | 0.1 |
| Thyroid | 0.0 | Renal ca. ACHN | 0.4 |
| Salivary gland | 0.0 | Renal ca. UO-31 | 0.0 |
| Pituitary gland | 0.1 | Renal ca. TK-10 | 0.0 |
| Brain (fetal) | 0.4 | Liver | 0.0 |
| Brain (whole) | 0.2 | Liver (fetal) | 0.0 |
| Brain (amygdala) | 0.1 | Liver ca. | 0.1 |
| | | (hepatoblast) HepG2 | |
| Brain (cerebellum) | 0.5 | Lung | 0.0 |
| Brain (hippocampus) | 0.1 | Lung (fetal) | 0.1 |
| Brain (substantia nigra) | 0.1 | Lung ca. (small cell) | 0.1 |
| | | LX-1 | |
| Brain (thalamus) | 0.1 | Lung ca. (small cell) | 0.0 |
| | | NCI-H69 | |
| Cerebral Cortex | 0.1 | Lung ca. (s.cell var.) | 0.0 |
| | | SHP-77 | |
| Spinal cord | 0.1 | Lung ca. (large | 0.0 |
| | | cell) NCI-H460 | |
| (glio/astro) U87-MG | 0.0 | Lung ca. (non-sm. | 0.2 |
| | | cell) A549 | |
| (glio/astro) U-118-MG | 0.0 | Lung ca. (non-s.cell) | 0.4 |
| | | NCI-H23 | |
| astrocytoma SW1783 | 0.1 | Lung ca. (non-s.cell) | 0.1 |
| | | HOP-62 | |
| neuro*; met SK-N-AS | 0.1 | Lung ca. (non-s.cl) | 0.3 |
| | | NCI-H522 | |
| astrocytoma SF-539 | 0.0 | Lung ca. (squam.) | 0.2 |
| | | SW 900 | |
| astrocytoma SNB-75 | 0.2 | Lung ca. (squam.) | 0.0 |
| | | NCI-H596 | |
| glioma SNB-19 | 0.1 | Mammary gland | 0.0 |
| glioma U251 | 0.1 | Breast ca.* (pl.ef) | 0.0 |
| | | MCF-7 | |
| glioma SF-295 | 0.1 | Breast ca.* (pl.ef) | 0.0 |
| | | MDA-MB-231 | |
| Heart (fetal) | 0.0 | Breast ca.* (pl.ef) | 0.2 |
| | | T47D | |
| Heart | 0.1 | Breast ca. BT-549 | 0.0 |
| Skeletal muscle (fetal) | 0.1 | Breast ca. MDA-N | 0.0 |
| Skeletal muscle | 0.1 | Ovary | 0.1 |
| Bone marrow | 0.0 | Ovarian ca. OVCAR-3 | 0.1 |
| Thymus | 0.1 | Ovarian ca. OVCAR-4 | 0.1 |
| Spleen | 0.0 | Ovarian ca. OVCAR-5 | 0.3 |
| Lymph node | 0.0 | Ovarian ca. OVCAR-8 | 0.1 |
| Colorectal | 0.0 | Ovarian ca. IGROV-1 | 0.0 |
| Stomach | 0.1 | Ovarian ca* (ascites) | 0.1 |
| | | SK-OV-3 | |
| Small intestine | 0.0 | Uterus | 0.0 |
| Colon ca. SW480 | 0.0 | Placenta | 0.0 |
| Colon ca.* | 0.2 | Prostate | 0.0 |
| SW620 (SW480 met) | | | |
| Colon ca. HT29 | 0.0 | Prostate ca.* (bone | 0.0 |
| | | met)PC-3 | |
| Colon ca. HCT-116 | 0.0 | Testis | 0.1 |
| Colon ca. CaCo-2 | 0.2 | Melanoma | 0.0 |
| | | Hs688(A).T | |
| Colon ca. | 0.0 | Melanoma* (met) | 0.0 |
| tissue (ODO3866) | | Hs688(B).T | |
| Colon ca. HCC-2998 | 0.1 | Melanoma UACC-62 | 0.0 |
| Gastric ca.* (liver met) | 0.1 | Melanoma M14 | 0.0 |
| NCI-N87 | | | |
| Bladder | 0.1 | Melanoma LOX | 0.0 |
| | | IMVI | |
| Trachea | 100.0 | Melanoma* (met) | 0.0 |
| | | SK-MEL-5 | |
| Kidney | 0.2 | Adipose | 0.0 |

TABLE BUC
Panel 4D

| | Rel. Exp. (%) | | Rel. Exp. (%) |
|---|---|---|---|
| | Ag3044, Run | | Ag3044, Run |
| Tissue Name | 164334372 | Tissue Name | 164334372 |
| Secondary Th1 act | 0.0 | HUVEC IL-1beta | 3.5 |
| Secondary Th2 act | 5.1 | HUVEC IFN gamma | 0.0 |
| Secondary Tr1 act | 1.3 | HUVEC TNF alpha + IFN | 2.0 |
| | | gamma | |
| Secondary Th1 rest | 1.2 | HUVEC TNF alpha + IL4 | 2.0 |
| Secondary Th2 rest | 0.7 | HUVEC IL-11 | 0.0 |
| Secondary Tr1 rest | 2.1 | Lung Microvascular EC | 1.6 |
| | | none | |
| Primary Th1 act | 2.0 | Lung Microvascular EC | 0.3 |
| | | TNF alpha + IL-1beta | |
| Primary Th2 act | 4.0 | Microvascular Dermal EC | 1.6 |
| | | none | |
| Primary Tr1 act | 0.0 | Microsvascular Dermal EC | 2.7 |
| | | TNF alpha + IL-1beta | |
| Primary Th1 rest | 9.5 | Bronchial epithelium | 6.7 |
| | | TNF alpha + IL1beta | |
| Primary Th2 rest | 11.9 | Small airway epithelium | 2.5 |
| | | none | |
| Primary Tr1 rest | 4.6 | Small airway epithelium | 20.9 |
| | | TNF alpha + IL-1beta | |
| CD45RA CD4 | 0.0 | Coronery artery SMC rest | 4.3 |
| lymphocyte act | | | |
| CD45RO CD4 | 1.5 | Coronery artery SMC | 0.6 |
| lymphocyte act | | TNF alpha + IL-1beta | |
| CD8 lymphocyte act | 5.3 | Astrocytes rest | 4.3 |
| Secondary CD8 | 1.9 | Astrocytes TNF alpha + | 2.9 |
| lymphocyte rest | | IL-1beta | |
| Secondary CD8 | 22.8 | KU-812 (Basophil) rest | 6.8 |
| lymphocyte act | | | |
| CD4 lymphocyte none | 0.1 | KU-812 (Basophil) | 9.5 |
| | | PMA/ionomycin | |
| 2ry Th1/Th2/Tr1_anti- | 2.5 | CCD1106 (Keratinocytes) | 2.1 |
| CD95 CH11 | | none | |
| LAK cells rest | 4.5 | CCD1106 (Keratinocytes) | 0.0 |
| | | TNF alpha + IL-1beta | |
| LAK cells IL-2 | 0.0 | Liver cirrhosis | 17.7 |
| LAK cells IL-2 + IL-12 | 2.9 | Lupus kidney | 8.1 |
| LAK cells IL-2 + IFN | 8.6 | NCI-H292 none | 52.5 |
| gamma | | | |
| LAK cells IL-2 + IL-18 | 6.5 | NCI-H292 IL-4 | 20.6 |
| LAK cells | 0.2 | NCI-H292 IL-9 | 31.4 |
| PMA/ionomycin | | | |
| NK Cells IL-2 rest | 2.1 | NCI-H292 IL-13 | 16.3 |
| Two Way MLR 3 day | 1.8 | NCI-H292 IFN gamma | 11.9 |
| Two Way MLR 5 day | 0.0 | HPAEC none | 0.0 |
| Two Way MLR 7 day | 0.0 | HPAEC TNF alpha + IL-1 | 1.0 |
| | | beta | |
| PBMC rest | 4.6 | Lung fibroblast none | 3.8 |
| PBMC PWM | 3.2 | Lung fibroblast TNF | 3.6 |
| | | alpha + IL-1beta | |
| PBMC PHA-L | 3.0 | Lung fibroblast IL-4 | 5.6 |
| Ramos (B cell) none | 0.0 | Lung fibroblast IL-9 | 6.9 |
| Ramos (B cell) | 0.2 | Lung fibroblast IL-13 | 2.9 |
| ionomycin | | | |
| B lymphocytes PWM | 11.7 | Lung fibroblast IFN | 3.0 |
| | | gamma | |
| B lymphocytes CD40L | 86.5 | Dermal fibroblast | 5.8 |
| and IL-4 | | CCD1070 rest | |
| EOL-1 dbcAMP | 0.0 | Dermal fibroblast | 9.9 |
| | | CCD1070 TNF alpha | |
| EOL-1 dbcAMP | 0.0 | Dermal fibroblast | 0.9 |
| PMA/ionomycin | | CCD1070 IL-1beta | |
| Dendritic cells none | 4.1 | Dermal fibroblast IFN | 1.0 |
| | | gamma | |
| Dendritic cells LPS | 1.1 | Dermal fibroblast IL-4 | 4.6 |
| Dendritic cells anti- | 1.1 | IBD Colitis 2 | 0.0 |
| CD40 | | | |
| Monocytes rest | 1.7 | IBD Crohn's | 3.8 |
| Monocytes LPS | 2.5 | Colon | 17.9 |
| Macrophages rest | 4.1 | Lung | 7.6 |
| Macrophages LPS | 0.0 | Thymus | 100.0 |
| HUVEC none | 4.8 | Kidney | 10.1 |
| HUVEC starved | 2.2 | | |

Panel 1.3D Summary: Ag3044 Results from one experiment with the NOV80 gene are not included. The amp plot indicates that there were experimental difficulties with this run (data not shown).

Panel 4D Summary: Ag3044 The NOV80 gene is expressed at low levels in a wide range of cell types of significance in the immune response in health and disease. These cells include: (i) resting LAK and LAK cells stimulated with IL-2+IL-12, IL-2+IL-18, and IL-2+IFNgamma (ii) activated primary and secondary Th2 cells, resting primary Th1, Th2 and Tr1 cells, and activated CD8 and secondary CD8 lymphocyte, (iii) IL-1 beta treated HUVECs, (iv) polkweed mitogen stimulated and CD40L+IL-4 stimulated B lymphocytes, (v) treated and non-treated Ku-812 basophils and non-treated dendritic cells, (vi) treated and non-treated peripheral blood mononuclear cells and resting macrophages (vii) treated and non-treated NCI-H292 mucoepidermoid, (viii) treated and non-treated lung fibroblasts, (viii) treated and non-treated astrocytes (ix) resting coronery artery SMCs, (x) resting and TNFalpha treated CCD1070 dermal fibroblasts and IL-4 treated dermal fibroblasts (xi) IBD Crohn's diseases tissue and normal tissues represented by colon, lung, thymus and kidney with the highest expression being detected in thymus tissue (CT=29.81). This expression profile suggests that this gene product may be involved in both disease and homeostatic processes in these and other cell types and tissues. Therefore, modulation of this gene product with a functional therapeutic may lead to the alteration of functions associated with these cell and tissue types and lead to improvement of the symptoms of patients suffering from autoimmune and inflammatory diseases such as COPD, emphysema, asthma, allergies, inflammatory bowel disease, lupus erythematosus, psoriasis, rheumatoid arthritis, and osteoarthritis.

NOV81a and NOV81b

Expression of gene NOV81a and the full length clone, NOV81b, was assessed using the primer-probe set Ag2906, described in Table BVA. Results of the RTQ-PCR runs are shown in Tables BVB, BVC and BVD.

TABLE BVA
Probe Name Ag2906

| | | | Start | |
|---|---|---|---|---|
| Primers | Sequences | Length | Position | SEQ ID NO: |
| Forward | 5′-ctacctgggtgaggtctttacc-3′ | 22 | 845 | 1318 |
| Probe | TET-5′-ctccggaagccaggaggaccctt-3′-TAMRA | 23 | 879 | 1319 |
| Reverse | 5′-agaaggactcgggcacatag-3′ | 20 | 902 | 1320 |

TABLE BVB
Panel 1.3D

| | Rel. Exp. (%) Ag2906, | | Rel. Exp. (%) Ag2906, |
|---|---|---|---|
| Tissue Name | Run 162556445 | Tissue Name | Run 162556445 |
| Liver adenocarcinoma | 2.0 | Kidney (fetal) | 13.5 |
| Pancreas | 1.2 | Renal ca. 786-0 | 1.0 |
| Pancreatic ca. CAPAN 2 | 0.0 | Renal ca. A498 | 2.8 |
| Adrenal gland | 1.1 | Renal ca. RXF 393 | 1.9 |
| Thyroid | 4.0 | Renal ca. ACHN | 0.7 |
| Salivary gland | 3.3 | Renal ca. UO-31 | 0.0 |
| Pituitary gland | 2.4 | Renal ca. TK-10 | 0.0 |
| Brain (fetal) | 0.0 | Liver | 0.0 |
| Brain (whole) | 0.0 | Liver (fetal) | 6.8 |
| Brain (amygdala) | 2.0 | Liver ca. | 5.4 |
| | | (hepatoblast) HepG2 | |
| Brain (cerebellum) | 3.8 | Lung | 42.0 |
| Brain (hippocampus) | 4.7 | Lung (fetal) | 11.8 |
| Brain (substantia nigra) | 0.9 | Lung ca. (small cell) | 0.7 |
| | | LX-1 | |
| Brain (thalamus) | 4.6 | Lung ca. (small cell) | 1.0 |
| | | NCI-H69 | |
| Cerebral Cortex | 6.4 | Lung ca. (s.cell var.) | 4.5 |
| | | SHP-77 | |
| Spinal cord | 7.6 | Lung ca. (large | 0.0 |
| | | cell) NCI-H460 | |
| glio/astro U87-MG | 3.0 | Lung ca. (non-sm. | 2.1 |
| | | cell) A549 | |
| glio/astro U-118-MG | 0.0 | Lung ca. (non-s.cell) | 1.6 |
| | | NCI-H23 | |
| astrocytoma SW1783 | 4.1 | Lung ca. (non-s.cell) | 4.0 |
| | | HOP-62 | |
| neuro*; met SK-N-AS | 0.0 | Lung ca. (non-s.cl) | 0.0 |
| | | NCI-H522 | |
| astrocytoma SF-539 | 4.5 | Lung ca. (squam.) | 8.4 |
| | | SW 900 | |
| astrocytoma SNB-75 | 4.9 | Lung ca. (squam.) | 0.2 |
| | | NCI-H596 | |
| glioma SNB-19 | 7.2 | Mammary gland | 4.0 |
| glioma U251 | 3.2 | Breast ca.* (pl.ef) | 0.0 |
| | | MCF-7 | |
| glioma SF-295 | 4.0 | Breast ca.* (pl.ef) | 3.9 |
| | | MDA-MB-231 | |
| Heart (fetal) | 100.0 | Breast ca.* (pl.ef) | 1.0 |
| | | T47D | |
| Heart | 10.7 | Breast ca. BT-549 | 1.8 |
| Skeletal muscle (fetal) | 35.8 | Breast ca. MDA-N | 0.3 |
| Skeletal muscle | 0.3 | Ovary | 2.2 |
| Bone marrow | 20.6 | Ovarian ca. OVCAR-3 | 1.8 |
| Thymus | 15.8 | Ovarian ca. OVCAR-4 | 0.6 |
| Spleen | 13.8 | Ovarian ca. OVCAR-5 | 0.8 |
| Lymph node | 5.5 | Ovarian ca. OVCAR-8 | 0.0 |
| Colorectal | 31.2 | Ovarian ca. IGROV-1 | 0.0 |
| Stomach | 7.3 | Ovarian ca.* (ascites) | 0.0 |
| | | SK-OV-3 | |
| Small intestine | 31.9 | Uterus | 4.8 |
| Colon ca. SW480 | 1.0 | Placenta | 9.9 |
| Colon ca.* | 0.0 | Prostate | 9.8 |
| SW620 (SW480 met) | | | |
| Colon ca. HT29 | 1.6 | Prostate ca.* (bone | 0.0 |
| | | met)PC-3 | |
| Colon ca. HCT-116 | 1.5 | Testis | 2.4 |
| Colon ca. CaCo-2 | 4.4 | Melanoma | 1.0 |
| | | Hs688(A).T | |
| Colon ca. | 8.7 | Melanoma* (met) | 2.6 |
| tissue (ODO3866) | | Hs688(B).T | |
| Colon ca. HCC-2998 | 8.8 | Melanoma UACC-62 | 0.0 |
| Gastric ca.* (liver met) | 2.3 | Melanoma M14 | 0.0 |
| NCI-N87 | | | |
| Bladder | 9.4 | Melanoma LOX | 0.0 |
| | | IMVI | |
| Trachea | 14.1 | Melanoma* (met) | 2.1 |
| | | SK-MEL-5 | |
| Kidney | 11.8 | Adipose | 6.9 |

TABLE BVC
Panel 2D

| | Rel. Exp. (%) | | Rel. Exp. (%) |
|---|---|---|---|
| | Ag2906, Run | | Ag2906, Run |
| Tissue Name | 162345752 | Tissue Name | 162345752 |
| Normal Colon | 39.5 | Kidney Margin | 3.0 |
| | | 8120608 | |
| CC Well to Mod Diff | 16.3 | Kidney Cancer | 17.3 |
| (ODO3866) | | 8120613 | |
| CC Margin (ODO3866) | 33.9 | Kidney Margin | 6.5 |
| | | 8120614 | |
| CC Gr.2 rectosigmoid (ODO3868) | 14.4 | Kidney Cancer | 3.4 |
| | | 9010320 | |
| CC Margin (ODO3868) | 1.2 | Kidney Margin | 7.2 |
| | | 9010321 | |
| CC Mod Diff (ODO3920) | 27.9 | Normal Uterus | 4.0 |
| CC Margin (ODO3920) | 30.8 | Uterus Cancer 064011 | 4.3 |
| CC Gr.2 ascend colon | 100.0 | Normal Thyroid | 2.6 |
| (ODO3921) | | | |
| CC Margin (ODO3921) | 33.9 | Thyroid Cancer | 3.3 |
| | | 064010 | |
| CC from Partial | 16.2 | Thyroid Cancer | 10.7 |
| Hepatectomy (ODO4309) | | A302152 | |
| Mets | | | |
| Liver Margin (ODO4309) | 2.9 | Thyroid Margin | 5.5 |
| | | A302153 | |
| Colon mets to lung | 15.6 | Normal Breast | 7.5 |
| (OD04451-01) | | | |
| Lung Margin (OD04451- | 15.8 | Breast Cancer | 7.4 |
| 02) | | (OD04566) | |
| Normal Prostate 6546-1 | 24.5 | Breast Cancer | 11.0 |
| | | (OD04590-01) | |
| Prostate Cancer | 4.8 | Breast Cancer Mets | 30.8 |
| (OD04410) | | (OD04590-03) | |
| Prostate Margin | 5.0 | Breast Cancer | 7.0 |
| (OD04410) | | Metastasis | |
| | | (OD04655-05) | |
| Prostate Cancer | 9.5 | Breast Cancer 064006 | 5.7 |
| (OD04720-01) | | | |
| Prostate Margin | 19.5 | Breast Cancer 1024 | 7.5 |
| (OD04720-02) | | | |
| Normal Lung 061010 | 16.5 | Breast Cancer | 13.7 |
| | | 9100266 | |
| Lung Met to Muscle | 3.9 | Breast Margin | 2.7 |
| (ODO4286) | | 9100265 | |
| Muscle Margin | 6.2 | Breast Cancer | 6.1 |
| (ODO4286) | | A209073 | |
| Lung Malignant Cancer | 64.2 | Breast Margin | 5.6 |
| (OD03126) | | A2090734 | |
| Lung Margin (OD03126) | 29.1 | Normal Liver | 1.9 |
| Lung Cancer (OD04404) | 16.7 | Liver Cancer 064003 | 0.4 |
| Lung Margin (OD04404) | 15.1 | Liver Cancer 1025 | 0.6 |
| Lung Cancer (OD04565) | 27.7 | Liver Cancer 1026 | 2.0 |
| Lung Margin (OD04565) | 19.8 | Liver Cancer 6004-T | 0.9 |
| Lung Cancer (OD04237- | 4.5 | Liver Tissue 6004-N | 2.8 |
| 01) | | | |
| Lung Margin (OD04237- | 12.9 | Liver Cancer 6005-T | 0.4 |
| 02) | | | |
| Ocular Mel Met to Liver | 1.8 | Liver Tissue 6005-N | 1.0 |
| (ODO4310) | | | |
| Liver Margin (ODO4310) | 2.6 | Normal Bladder | 8.5 |
| Melanoma Mets to Lung | 4.2 | Bladder Cancer 1023 | 2.4 |
| (OD04321) | | | |
| Lung Margin (OD04321) | 35.6 | Bladder Cancer | 0.8 |
| | | A302173 | |
| Normal Kidney | 5.4 | Bladder Cancer | 5.8 |
| | | (OD04718-01) | |
| Kidney Ca, Nuclear grade | 4.1 | Bladder Normal | 2.0 |
| 2 (OD04338) | | Adjacent (OD04718- | |
| | | 03) | |
| Kidney Margin | 11.0 | Normal Ovary | 3.1 |
| (OD04338) | | | |
| Kidney Ca Nuclear grade | 5.0 | Ovarian Cancer | 11.0 |
| 1/2 (OD04339) | | 064008 | |
| Kidney Margin | 2.0 | Ovarian Cancer | 7.9 |
| (OD04339) | | (OD04768-07) | |
| Kidney Ca, Clear cell | 3.8 | Ovary Margin | 1.3 |
| type (OD04340) | | (OD04768-08) | |
| Kidney Margin | 5.6 | Normal Stomach | 2.9 |
| (OD04340) | | | |
| Kidney Ca, Nuclear grade | 2.7 | Gastric Cancer | 1.0 |
| 3 (OD04348) | | 9060358 | |
| Kidney Margin | 5.0 | Stomach Margin | 3.7 |
| (OD04348) | | 9060359 | |
| Kidney Cancer | 5.9 | Gastric Cancer | 12.0 |
| (OD04622-01) | | 9060395 | |
| Kidney Margin | 2.1 | Stomach Margin | 14.2 |
| (OD04622-03) | | 9060394 | |
| Kidney Cancer | 0.8 | Gastric Cancer | 31.9 |
| (OD04450-01) | | 9060397 | |
| Kidney Margin | 4.5 | Stomach Margin | 5.6 |
| (OD04450-03) | | 9060396 | |
| Kidney Cancer 8120607 | 14.6 | Gastric Cancer | 8.7 |
| | | 064005 | |

TABLE BVD
Panel 4D

| | Rel. Exp. (%) | | Rel. Exp. (%) |
|---|---|---|---|
| | Ag2906, Run | | Ag2906, Run |
| Tissue Name | 159078634 | Tissue Name | 159078634 |
| Secondary Th1 act | 5.6 | HUVEC IL-1beta | 5.7 |
| Secondary Th2 act | 5.3 | HUVEC IFN gamma | 9.2 |
| Secondary Tr1 act | 8.6 | HUVEC TNF alpha + IFN | 9.5 |
| | | gamma | |
| Secondary Th1 rest | 11.0 | HUVEC TNF alpha + IL4 | 4.6 |
| Secondary Th2 rest | 6.6 | HUVEC IL-11 | 9.1 |
| Secondary Tr1 rest | 10.5 | Lung Microvascular EC | 10.0 |
| | | none | |
| Primary Th1 act | 2.0 | Lung Microvascular EC | 3.8 |
| | | TNF alpha + IL-1beta | |
| Primary Th2 act | 1.2 | Microvascular Dermal EC | 10.2 |
| | | none | |
| Primary Tr1 act | 1.1 | Microsvasular Dermal EC | 2.5 |
| | | TNF alpha + IL-1beta | |
| Primary Th1 rest | 10.7 | Bronchial epithelium | 20.4 |
| | | TNF alpha + IL1beta | |
| Primary Th2 rest | 4.4 | Small airway epithelium | 8.0 |
| | | none | |
| Primary Tr1 rest | 18.0 | Small airway epithelium | 22.4 |
| | | TNF alpha + IL-1beta | |
| CD45RA CD4 | 8.0 | Coronery artery SMC rest | 4.0 |
| lymphocyte act | | | |
| CD45RO CD4 | 5.1 | Coronery artery SMC | 6.1 |
| lymphocyte act | | TNF alpha + IL-1beta | |
| CD8 lymphocyte act | 3.7 | Astrocytes rest | 7.4 |
| Secondary CD8 | 2.5 | Astrocytes TNF alpha + | 3.1 |
| lymphocyte rest | | IL-1beta | |
| Secondary CD8 | 1.5 | KU-812 (Basophil) rest | 2.6 |
| lymphocyte act | | | |
| CD4 lymphocyte none | 6.5 | KU-812 (Basophil) | 2.7 |
| | | PMA/ionomycin | |
| 2ry Th1/Th2/Tr1_anti- | 11.6 | CCD1106 (Keratinocytes) | 6.4 |
| CD95 CH11 | | none | |
| LAK cells rest | 14.6 | CCD1106 (Keratinocytes) | 3.6 |
| | | TNF alpha + IL-1beta | |
| LAK cells IL-2 | 8.6 | Liver cirrhosis | 8.0 |
| LAK cells IL-2 + IL-12 | 6.3 | Lupus kidney | 2.5 |
| LAK cells IL-2 + IFN | 14.8 | NCI-H292 none | 3.7 |
| gamma | | | |
| LAK cells IL-2 + IL-18 | 2.5 | NCI-H292 IL-4 | 4.9 |
| LAK cells | 7.6 | NCI-H292 IL-9 | 0.7 |
| PMA/ionomycin | | | |
| NK Cells IL-2 rest | 11.2 | NCI-H292 IL-13 | 1.4 |
| Two Way MLR 3 day | 6.4 | NCI-H292 IFN gamma | 1.3 |
| Two Way MLR 5 day | 8.3 | HPAEC none | 4.0 |
| Two Way MLR 7 day | 2.7 | HPAEC TNF alpha + IL-1 | 8.0 |
| | | beta | |
| PBMC rest | 6.2 | Lung fibroblast none | 5.3 |
| PBMC PWM | 8.2 | Lung fibroblast TNF | 0.0 |
| | | alpha +IL-1beta | |
| PBMC PHA-L | 2.6 | Lung fibroblast IL-4 | 1.6 |
| Ramos (B cell) none | 7.5 | Lung fibroblast IL-9 | 2.0 |
| Ramos (B cell) | 2.2 | Lung fibroblast IL-13 | 3.6 |
| ionomycin | | | |
| B lymphocytes PWM | 2.4 | Lung fibroblast IFN | 2.7 |
| | | gamma | |
| B lymphocytes CD40L | 100.0 | Dermal fibroblast | 2.6 |
| and IL-4 | | CCD1070 rest | |
| EOL-1 dbcAMP | 44.4 | Dermal fibroblast | 8.5 |
| | | CCD1070 TNF alpha | |
| EOL-1 dbcAMP | 77.9 | Dermal fibroblast | 5.6 |
| PMA/ionomycin | | CCD1070 IL-1beta | |
| Dendritic cells none | 21.2 | Dermal fibroblast IFN | 5.7 |
| | | gamma | |
| Dendritic cells LPS | 3.0 | Dermal fibroblast IL-4 | 9.0 |
| Dendritic cells anti- | 9.9 | IBD Colitis 2 | 0.0 |
| CD40 | | | |
| Monocytes rest | 28.1 | IBD Crohn's | 2.6 |
| Monocytes LPS | 10.3 | Colon | 97.9 |
| Macrophages rest | 33.0 | Lung | 41.5 |
| Macrophages LPS | 12.7 | Thymus | 5.5 |
| HUVEC none | 11.9 | Kidney | 6.9 |
| HUVEC starved | 20.4 | | |

Panel 1.3D Summary: Ag2906 The NOV81a gene has a low level of expression in adipose and may be a small molecule target for the treatment of obesity and obesity-related diseases, including Type 2 diabetes. In addition, this gene product appears to be differentially expressed in fetal (CT value=31) vs adult heart (CT value=34) and may be useful for the differentiation between the two tissue types.

Overall, there appears to be higher expression of this gene in the normal tissues compared to the cell lines. Thus, this difference in expression might be of use as a diagnostic marker of cancer.

Panel 2D Summary: Ag2906 The NOV81a gene is expressed at low levels in this panel. A higher level of expression is observed in gastric, bladder, thyroid, breast and ovarian cancer samples when compared to expression in the normal adjacent gastric, bladder, thyroid, breast and ovary tissues. Thus, this gene could potentially be used as a diagnostic marker of cancer in these tissues. Furthermore, inhibition of the activity of this gene product using small molecule drugs may be useful for the treatment of cancer in these tissues.

Panel 4D Summary: Ag2906 Expression of the NOV81a is widespread in this panel, with highest expression in B lymphocytes treated with CD40L and IL-4 (CT=29.8). Significant expression is also seen in treated eosinophils, resting macrophages and monocytes, and normal colon and lung. Based on this pattern of expression, this gene product may be involved in both disease and homeostatic processes for these and other cell types and tissues. Therefore, modulation of this gene product with a functional therapeutic may lead to the symptoms of patients suffering from autoimmune and inflammatory diseases such as COPD, emphysema, asthma, allergies, inflammatory bowel disease, lupus erythematosus, psoriasis, rheumatoid arthritis, and osteoarthritis. In addition, the higher levels of expression in colon (CT=30) when compared to colon from patients with inflammatory bowel diseases (IBD)(CTs=35-40) suggests that expression of this gene could be used to differentiate between normal and inflamed colon. Therapeutic modulation of the expression or function of this gene may be effective in the treatment of IBD.

NOV82

Expression of gene NOV82 was assessed using the primer-probe sets Ag3198 and Ag3063, described in Tables BWA and BWB.

TABLE BWA
Probe Name Ag3198

| | | | Start | |
|---|---|---|---|---|
| Primers | Sequences | Length | Position | SEQ ID NO: |
| Forward | 5′-cgtggtcaccagacagttaatt-3′ | 22 | 179 | 1321 |
| Probe | TET-5′-cctaccagacaccattgtgtccaagg-3′-TAMRA | 26 | 212 | 1322 |
| Reverse | 5′-gtctttcctttgtgcttgtgaa-3′ | 22 | 246 | 1323 |

TABLE BWB
Probe Name Ag3063

| | | | Start | |
|---|---|---|---|---|
| Primers | Sequences | Length | Position | SEQ ID NO: |
| Forward | 5′-cgtggtcaccagacagttaatt-3′ | 22 | 179 | 1324 |
| Probe | TET-5′-cctaccagacaccattgtgtccaagg-3′-TAMRA | 26 | 212 | 1325 |
| Reverse | 5′-gtctttcctttgtgcttgtgaa-3′ | 22 | 246 | 1326 |

CNS_neurodegeneration_v1.0 Summary: Ag3l98 Expression of this gene is low/undetectable (CTs>35) across all of the samples on this panel (data not shown). The amp plot indicates that there is a high probability of a potential probe or chemistry failure.

Panel 1.3D Summary: Ag3063 Expression of this gene is low/undetectable (CTs>35) across all of the samples on this panel (data not shown). The amp plot indicates that there is a high probability of a potential probe or chemistry failure.

Panel 4D Summary: Ag3198 Expression of this gene is low/undetectable (CTs>35) across all of the samples on this panel (data not shown). The amp plot indicates that there is a high probability of a potential probe or chemistry failure.

NOV83

Expression of gene NOV83 was assessed using the primer-probe sets Ag3046 and Ag4125, described in Tables BXA and BXB. Results of the RTQ-PCR runs are shown in Tables BXC and BXD.

TABLE BXA
Probe Name Ag3046

| | | | Start | |
|---|---|---|---|---|
| Primers | Sequences | Length | Position | SEQ ID NO: |
| Forward | 5′-gaagcaaagaactctgcaagac-3′ | 22 | 1215 | 1327 |
| Probe | TET-5′-ttccagcatgataacttcacagagga-3′-TAMRA | 26 | 1246 | 1328 |
| Reverse | 5′-gagcctgcaaatatcttttgct-3′ | 22 | 1272 | 1329 |

TABLE BXB
Probe Name Ag4125

| | | | Start | |
|---|---|---|---|---|
| Primers | Sequences | Length | Position | SEQ ID NO: |
| Forward | 5′-gaagcaaagaactctgcaagac-3′ | 22 | 1215 | 1330 |
| Probe | TET-5′-ttccagcatgataacttcacagagga-3′-TAMRA | 26 | 1246 | 1331 |
| Reverse | 5′-gagcctgcaaatatcttttgct-3′ | 22 | 1272 | 1332 |

TABLE BXC
Panel 2D

| | Rel. Exp. (%) | | Rel. Exp. (%) |
|---|---|---|---|
| | Ag3046, Run | | Ag3046, Run |
| Tissue Name | 162559104 | Tissue Name | 162559104 |
| Normal Colon | 0.0 | Kidney Margin | 0.0 |
| | | 8120608 | |
| CC Well to Mod Diff | 0.0 | Kidney Cancer | 0.0 |
| (ODO3866) | | 8120613 | |
| CC Margin (ODO3866) | 0.0 | Kidney Margin | 0.0 |
| | | 8120614 | |
| CC Gr.2 rectosigmoid (ODO3868) | 0.0 | Kidney Cancer | 0.0 |
| | | 9010320 | |
| CC Margin (ODO3868) | 0.0 | Kidney Margin | 0.0 |
| | | 9010321 | |
| CC Mod Diff (ODO3920) | 0.0 | Normal Uterus | 0.0 |
| CC Margin (ODO3920) | 0.0 | Uterus Cancer 064011 | 0.0 |
| CC Gr.2 ascend colon | 0.0 | Normal Thyroid | 0.0 |
| (ODO3921) | | | |
| CC Margin (ODO3921) | 0.0 | Thyroid Cancer | 0.0 |
| | | 064010 | |
| CC from Partial | 0.0 | Thyroid Cancer | 0.0 |
| Hepatectomy (ODO4309) | | A302152 | |
| Mets | | | |
| Liver Margin (ODO4309) | 0.0 | Thyroid Margin | 0.0 |
| | | A302153 | |
| Colon mets to lung | 0.0 | Normal Breast | 0.0 |
| (OD04451-01) | | | |
| Lung Margin (OD04451- | 0.0 | Breast Cancer | 100.0 |
| 02) | | (OD04566) | |
| Normal Prostate 6546-1 | 0.1 | Breast Cancer | 0.0 |
| | | (OD04590-01) | |
| Prostate Cancer | 0.0 | Breast Cancer Mets | 0.0 |
| (OD04410) | | (OD04590-03) | |
| Prostate Margin | 0.0 | Breast Cancer | 0.0 |
| (OD04410) | | Metastasis | |
| | | (OD04655-05) | |
| Prostate Cancer | 0.1 | Breast Cancer 064006 | 0.0 |
| (OD04720-01) | | | |
| Prostate Margin | 0.0 | Breast Cancer 1024 | 0.0 |
| (OD04720-02) | | | |
| Normal Lung 061010 | 0.0 | Breast Cancer | 0.0 |
| | | 9100266 | |
| Lung Met to Muscle | 0.0 | Breast Margin | 0.0 |
| (ODO4286) | | 9100265 | |
| Muscle Margin | 0.0 | Breast Cancer | 0.0 |
| (ODO4286) | | A209073 | |
| Lung Malignant Cancer | 0.0 | Breast Margin | 0.0 |
| (OD03126) | | A2090734 | |
| Lung Margin (OD03126) | 0.0 | Normal Liver | 0.0 |
| Lung Cancer (OD04404) | 0.0 | Liver Cancer 064003 | 0.0 |
| Lung Margin (OD04404) | 0.0 | Liver Cancer 1025 | 0.0 |
| Lung Cancer (OD04565) | 0.0 | Liver Cancer 1026 | 0.0 |
| Lung Margin (OD04565) | 0.0 | Liver Cancer 6004-T | 0.0 |
| Lung Cancer (OD04237- | 0.0 | Liver Tissue 6004-N | 0.0 |
| 01) | | | |
| Lung Margin (OD04237- | 0.0 | Liver Cancer 6005-T | 0.0 |
| 02) | | | |
| Ocular Mel Met to Liver | 0.0 | Liver Tissue 6005-N | 0.0 |
| (ODO4310) | | | |
| Liver Margin (ODO4310) | 0.0 | Normal Bladder | 0.0 |
| Melanoma Mets to Lung | 0.0 | Bladder Cancer 1023 | 0.0 |
| (OD04321) | | | |
| Lung Margin (OD04321) | 0.0 | Bladder Cancer | 0.0 |
| | | A302173 | |
| Normal Kidney | 0.0 | Bladder Cancer | 0.0 |
| | | (OD04718-01) | |
| Kidney Ca, Nuclear grade | 0.0 | Bladder Normal | 0.0 |
| 2 (OD04338) | | Adjacent (OD04718- | |
| | | 03) | |
| Kidney Margin | 0.0 | Normal Ovary | 0.0 |
| (OD04338) | | | |
| Kidney Ca Nuclear grade | 0.0 | Ovarian Cancer | 0.0 |
| 1/2 (OD04339) | | 064008 | |
| Kidney Margin | 0.0 | Ovarian Cancer | 0.0 |
| (OD04339) | | (OD04768-07) | |
| Kidney Ca, Clear cell | 0.0 | Ovary Margin | 0.0 |
| type (OD04340) | | (OD04768-08) | |
| Kidney Margin | 0.0 | Normal Stomach | 0.0 |
| (OD04340) | | | |
| Kidney Ca, Nuclear grade | 0.0 | Gastric Cancer | 0.0 |
| 3 (OD04348) | | 9060358 | |
| Kidney Margin | 0.0 | Stomach Margin | 0.0 |
| (OD04348) | | 9060359 | |
| Kidney Cancer | 0.0 | Gastric Cancer | 0.0 |
| (OD04622-01) | | 9060395 | |
| Kidney Margin | 0.0 | Stomach Margin | 0.0 |
| (OD04622-03) | | 9060394 | |
| Kidney Cancer | 0.0 | Gastric Cancer | 0.0 |
| (OD04450-01) | | 9060397 | |
| Kidney Margin | 0.0 | Stomach Margin | 0.0 |
| (OD04450-03) | | 9060396 | |
| Kidney Cancer 8120607 | 0.0 | Gastric Cancer | 0.0 |
| | | 064005 | |

TABLE BXD
Panel 4.1D

| | Rel. Exp. (%) | | Rel. Exp. (%) |
|---|---|---|---|
| | Ag4125, Run | | Ag4125, Run |
| Tissue Name | 172859315 | Tissue Name | 172859315 |
| Secondary Th1 act | 3.0 | HUVEC IL-1beta | 0.0 |
| Secondary Th2 act | 4.2 | HUVEC IFN gamma | 0.0 |
| Secondary Tr1 act | 0.0 | HUVEC TNF alpha + IFN | 0.0 |
| | | gamma | |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 2.7 | Lung Microvascular EC | 0.0 |
| | | none | |
| Primary Th1 act | 0.0 | Lung Microvascular EC | 0.0 |
| | | TNF alpha + IL-1beta | |
| Primary Th2 act | 3.0 | Microvascular Dermal EC | 0.0 |
| | | none | |
| Primary Tr1 act | 11.9 | Microsvasular Dermal EC | 7.4 |
| | | TNF alpha + IL-1beta | |
| Primary Th1 rest | 7.2 | Bronchial epithelium | 7.5 |
| | | TNF alpha + IL1beta | |
| Primary Th2 rest | 3.1 | Small airway epithelium | 0.0 |
| | | none | |
| Primary Tr1 rest | 0.0 | Small airway epithelium | 0.0 |
| | | TNF alpha + IL-1beta | |
| CD45RA CD4 | 0.0 | Coronery artery SMC rest | 0.0 |
| lymphocyte act | | | |
| CD45RO CD4 | 0.0 | Coronery artery SMC | 0.0 |
| lymphocyte act | | TNF alpha + IL-1beta | |
| CD8 lymphocyte act | 0.0 | Astrocytes rest | 0.0 |
| Secondary CD8 | 0.0 | Astrocytes TNF alpha + | 0.0 |
| lymphocyte rest | | IL-1beta | |
| Secondary CD8 | 0.0 | KU-812 (Basophil) rest | 0.0 |
| lymphocyte act | | | |
| CD4 lymphocyte none | 0.0 | KU-812 (Basophil) | 0.0 |
| | | PMA/ionomycin | |
| 2ry Th1/Th2/Tr1_anti- | 0.0 | CCD1106 (Keratinocytes) | 0.0 |
| CD95 CH11 | | none | |
| LAK cells rest | 3.3 | CCD1106 (Keratinocytes) | 0.0 |
| | | TNF alpha + IL-1beta | |
| LAK cells IL-2 | 0.0 | Liver cirrhosis | 0.0 |
| LAK cells IL-2 + IL-12 | 5.3 | NCI-H292 none | 0.0 |
| LAK cells IL-2 + IFN | 1.8 | NCI-H292 IL-4 | 5.8 |
| gamma | | | |
| LAK cells IL-2 + IL-18 | 3.6 | NCI-H292 IL-9 | 3.5 |
| LAK cells | 0.0 | NCI-H292 IL-13 | 1.3 |
| PMA/ionomycin | | | |
| NK Cells IL-2 rest | 5.9 | NCI-H292 IFN gamma | 0.8 |
| Two Way MLR 3 day | 3.3 | HPAEC none | 0.0 |
| Two Way MLR 5 day | 3.2 | HPAEC TNF alpha + IL-1 | 0.0 |
| | | beta | |
| Two Way MLR 7 day | 0.0 | Lung fibroblast none | 0.0 |
| PBMC rest | 0.0 | Lung fibroblast TNF alpha + | 2.3 |
| | | IL-1beta | |
| PBMC PWM | 3.0 | Lung fibroblast IL-4 | 0.0 |
| PBMC PHA-L | 1.6 | Lung fibroblast IL-9 | 0.0 |
| Ramos (B cell) none | 0.0 | Lung fibroblast IL-13 | 0.0 |
| Ramos (B cell) | 0.0 | Lung fibroblast IFN | 0.0 |
| ionomycin | | gamma | |
| B lymphocytes PWM | 0.0 | Dermal fibroblast | 1.0 |
| | | CCD1070 rest | |
| B lymphocytes CD40L | 0.0 | Dermal fibroblast | 0.0 |
| and IL-4 | | CCD1070 TNF alpha | |
| EOL-1 dbcAMP | 2.1 | Dermal fibroblast | 0.0 |
| | | CCD1070 IL-1beta | |
| EOL-1 dbcAMP | 3.3 | Dermal fibroblast IFN | 0.0 |
| PMA/ionomycin | | gamma | |
| Dendritic cells none | 0.0 | Dermal fibroblast IL-4 | 0.0 |
| Dendritic cells LPS | 0.0 | Dermal Fibroblasts rest | 0.0 |
| Dendritic cells anti- | 0.0 | Neutrophils TNFa + LPS | 0.0 |
| CD40 | | | |
| Monocytes rest | 0.0 | Neutrophils rest | 0.0 |
| Monocytes LPS | 13.5 | Colon | 0.0 |
| Macrophages rest | 0.0 | Lung | 5.4 |
| Macrophages LPS | 0.0 | Thymus | 18.4 |
| HUVEC none | 0.0 | Kidney | 100.0 |
| HUVEC starved | 0.0 | | |

CNS_neurodegeneration_v1.0 Summary: Ag3046 Expression of this gene is low/undetectable (CTs>35) across all of the samples on this panel (data not shown).

Ag4125 Results from one experiment with this gene are not included. The amp plot indicates that there were experimental difficulties with this run (data not shown).

General_screening_panel_v1.4 Summary: Ag4125 Expression of this gene is low/undetectable (CTs>35) across all of the samples on this panel (data not shown).

Panel 1.3D Summary: Ag3046 Expression of this gene is low/undetectable (CTs>35) across all of the samples on this panel (data not shown).

Panel 2D Summary: Ag3046 Significant expression of this gene is seen exclusively in a breast cancer sample (CT=25.2). Therefore, expression of this gene may be used to distinguish breast cancers from the other samples on this panel. Furthermore, therapeutic modulation of the activity of the GPCR encoded by this gene may be beneficial in the treatment of breast cancer.

Panel 3D Summary: Ag3046 Expression of this gene is low/undetectable (CTs>35) across all of the samples on this panel (data not shown).

Panel 4.1D Summary: Ag4125 This gene is only expressed at detectable levels in the kidney (CT=32.6). The putative GPCR encoded for by this gene could allow cells within the kidney to respond to specific microenvironmental signals (For example, ref. 1). Therefore, modulate kidney function and be important in the treatment of inflammatory or autoimmune diseases that affect the kidney, including lupus and glomerulonephritis.

References:

1. Mark M. D., Wittemann S., Herlitze S. (2000) G protein modulation of recombinant P/Q-type calcium channels by regulators of G protein signalling proteins. J. Physiol. 528 Pt 1: 65-77.

1. Fast synaptic transmission is triggered by the activation of presynaptic Ca2+ channels which can be inhibited by Gbetagamma subunits via G protein-coupled receptors (GPCR). Regulators of G protein signalling (RGS) proteins are GTPase-accelerating proteins (GAPs), which are responsible for >100-fold increases in the GTPase activity of G proteins and might be involved in the regulation of presynaptic Ca2+ channels. In this study we investigated the effects of RGS2 on G protein modulation of recombinant P/Q-type channels expressed in a human embryonic kidney (HEK293) cell line using whole-cell recordings. 2. Ba2+ currents (IBa) through P/Q-type channels heterologously expressed with the muscarinic facilitation properties of P/Q-type Ca2+ channels. G protein reinhibition is accelerated, while release from inhibition is slowed. These kinetics depend on the availability of G protein alpha and betagamma subunits which is altered by RGS proteins. 4. RGS proteins unmask the Ca2+ channel beta subunit modulation of Ca2+ channel G protein inhibition. In the presence of RGS2, P/Q-type channels containing the beta2a and beta3 subunits reveal significantly altered kinetics of G protein modulation and increased facilitation compared to Ca2+ channels coexpressed with the beta1b or beta4 subunit.

PMID: 11018106

Panel 4D Summary: Ag3046 Expression of this gene is low/undetectable (CTs>35) across all of the samples on this panel (data not shown).

NOV84

Expression of gene NOV84 was assessed using the primer-probe set Ag3051, described in Table BYA.

TABLE BYA
Probe Name Ag3051

| | | | Start | |
|---|---|---|---|---|
| Primers | Sequences | Length | Position | SEQ ID NO: |
| Forward | 5′-gcagctcattcagcctatgag-3′ | 22 | 847 | 1333 |
| Probe | TET-5′-ctctcctgccacccctatgacactg-3′-TAMRA | 25 | 883 | 1334 |
| Reverse | 5′-cgacaacaggtacatcatgaag-3′ | 22 | 913 | 1335 |

Panel 1.3D Summary: Ag3051 Results from one experiment with this gene are not included. The amp plot suggests that there were experimental difficulties with this run (data not shown).

Panel 2D Summary: Ag3051 Expression of this gene is low/undetectable (CTs>35) across all of the samples on this panel (data not shown).

Panel 4D Summary: Ag3051 Expression of this gene is low/undetectable (CTs>35) across all of the samples on this panel (data not shown).

NOV85

Expression of gene NOV85 was assessed using the primer-probe set Ag3057, described in Table BZA. Results of the RTQ-PCR runs are shown in Tables BZB, BZC, BZD and BZE.

TABLE BZA
Probe Name Ag3057

| | | | Start | |
|---|---|---|---|---|
| Primers | Sequences | Length | Position | SEQ ID NO: |
| Forward | 5′-aacattggaaggacaggagtct-3′ | 22 | 2314 | 1336 |
| Probe | TET-5′-ccccaggagatgtatcagattcagct-3′-TAMRA | 26 | 2336 | 1337 |
| Reverse | 5′-cagatccccaagaaccctta-3′ | 20 | 2382 | 1338 |

TABLE BZB
CNS_neurodegeneration_v1.0

| | Rel. Exp. (%) Ag3057, | | Rel. Exp. (%) Ag3057, |
|---|---|---|---|
| Tissue Name | Run 211012795 | Tissue Name | Run 211012795 |
| BAD 1 Hippo | 12.9 | Control (Path) 3 | 8.4 |
| | | Temporal Ctx | |
| AD 2 Hippo | 27.2 | Control (Path) 4 | 25.0 |
| | | Temporal Ctx | |
| AD 3 Hippo | 11.2 | AD 1 Occipital Ctx | 29.5 |
| AD 4 Hippo | 13.0 | AD 2 Occipital Ctx | 0.0 |
| | | (Missing) | |
| AD 5 hippo | 23.8 | AD 3 Occipital Ctx | 15.4 |
| AD 6 Hippo | 77.4 | AD 4 Occipital Ctx | 28.9 |
| Control 2 Hippo | 34.9 | AD 5 Occipital Ctx | 15.9 |
| Control 4 Hippo | 15.5 | AD 6 Occipital Ctx | 14.7 |
| Control (Path) 3 | 11.2 | Control 1 Occipital | 8.0 |
| Hippo | | Ctx | |
| AD 1 Temporal Ctx | 45.7 | Control 2 Occipital | 28.3 |
| | | Ctx | |
| AD 2 Temporal Ctx | 31.4 | Control 3 Occipital | 14.8 |
| | | Ctx | |
| AD 3 Temporal Ctx | 19.3 | Control 4 Occipital | 23.8 |
| | | Ctx | |
| AD 4 Temporal Ctx | 28.1 | Control (Path) 1 | 73.7 |
| | | Occipital Ctx | |
| AD 5 Inf Temporal | 41.2 | Control (Path) 2 | 16.8 |
| Ctx | | Occipital Ctx | |
| AD 5 SupTemporal | 23.3 | Control (Path) 3 | 7.3 |
| Ctx | | Occipital Ctx | |
| AD 6 Inf Temporal | 100.0 | Control (Path) 4 | 13.0 |
| Ctx | | Occipital Ctx | |
| AD6 Sup Temporal | 75.3 | Control 1 Parietal | 14.8 |
| Ctx | | Ctx | |
| Control 1 Temporal | 9.7 | Control 2 Parietal | 29.3 |
| Ctx | | Ctx | |
| Control 2 Temporal | 21.8 | Control 3 Parietal | 12.8 |
| Ctx | | Ctx | |
| Control 3 Temporal | 16.0 | Control (Path) 1 | 42.6 |
| Ctx | | Parietal Ctx | |
| Control 4 Temporal | 9.3 | Control (Path) 2 | 30.4 |
| Ctx | | Parietal Ctx | |
| Control (Path) 1 | 45.4 | Control (Path) 3 | 9.7 |
| Temporal Ctx | | Parietal Ctx | |
| Control (Path) 2 | 28.9 | Control (Path) 4 | 30.8 |
| Temporal Ctx | | Parietal Ctx | |

TABLE BZC
Panel 1.3D

| | Rel. Exp. (%) Ag3057, | | Rel. Exp. (%) Ag3057, |
|---|---|---|---|
| Tissue Name | Run 165519995 | Tissue Name | Run 165519995 |
| Liver adenocarcinoma | 9.9 | Kidney (fetal) | 6.1 |
| Pancreas | 3.8 | Renal ca. 786-0 | 8.8 |
| Pancreatic ca. CAPAN 2 | 40.9 | Renal ca. A498 | 15.1 |
| Adrenal gland | 14.5 | Renal ca. RXF 393 | 13.0 |
| Thyroid | 6.2 | Renal ca. ACHN | 2.1 |
| Salivary gland | 10.8 | Renal ca. UO-31 | 13.1 |
| Pituitary gland | 10.2 | Renal ca. TK-10 | 3.9 |
| Brain (fetal) | 51.4 | Liver | 1.9 |
| Brain (whole) | 100.0 | Liver (fetal) | 4.8 |
| Brain (amygdala) | 48.0 | Liver ca. | 17.3 |
| | | (hepatoblast) HepG2 | |
| Brain (cerebellum) | 49.3 | Lung | 10.4 |
| Brain (hippocampus) | 47.6 | Lung (fetal) | 7.2 |
| Brain (substantia nigra) | 70.2 | Lung ca. (small cell) | 7.6 |
| | | LX-1 | |
| Brain (thalamus) | 51.8 | Lung ca. (small cell) | 1.0 |
| | | NCI-H69 | |
| Cerebral Cortex | 11.3 | Lung ca. (s.cell var.) | 7.3 |
| | | SHP-77 | |
| Spinal cord | 85.9 | Lung ca. (large | 16.6 |
| | | cell)NCI-H460 | |
| glio/astro U87-MG | 16.5 | Lung ca. (non-sm. | 4.5 |
| | | cell) A549 | |
| glio/astro U-118-MG | 27.9 | Lung ca. (non-s.cell) | 6.1 |
| | | NCI-H23 | |
| astrocytoma SW1783 | 13.5 | Lung ca. (non-s.cell) | 8.5 |
| | | HOP-62 | |
| neuro*; met SK-N-AS | 12.2 | Lung ca. (non-s.cl) | 3.0 |
| | | NCI-H522 | |
| astrocytoma SF-539 | 14.2 | Lung ca. (squam.) | 8.3 |
| | | SW 900 | |
| astrocytoma SNB-75 | 20.9 | Lung ca. (squam.) | 5.9 |
| | | NCI-H596 | |
| glioma SNB-19 | 23.0 | Mammary gland | 17.3 |
| glioma U251 | 25.7 | Breast ca.* (pl.ef) | 7.3 |
| | | MCF-7 | |
| glioma SF-295 | 9.8 | Breast ca.* (pl.ef) | 35.8 |
| | | MDA-MB-231 | |
| Heart (fetal) | 0.2 | Breast ca.* (pl.ef) | 5.5 |
| | | T47D | |
| Heart | 11.1 | Breast ca. BT-549 | 32.1 |
| Skeletal muscle (fetal) | 3.2 | Breast ca. MDA-N | 3.3 |
| Skeletal muscle | 18.9 | Ovary | 1.9 |
| Bone marrow | 3.8 | Ovarian ca. OVCAR-3 | 3.8 |
| Thymus | 1.5 | Ovarian ca. OVCAR-4 | 6.9 |
| Spleen | 5.6 | Ovarian ca. OVCAR-5 | 7.7 |
| Lymph node | 7.3 | Ovarian ca. OVCAR-8 | 5.7 |
| Colorectal | 9.2 | Ovarian ca. IGROV-1 | 2.5 |
| Stomach | 15.0 | Ovarian ca.* (ascites) | 20.9 |
| | | SK-OV-3 | |
| Small intestine | 10.4 | Uterus | 23.5 |
| Colon ca. SW480 | 5.6 | Placenta | 6.0 |
| Colon ca.* | 4.9 | Prostate | 3.4 |
| SW620 (SW480 met) | | | |
| Colon ca. HT29 | 2.8 | Prostate ca.* (bone | 8.4 |
| | | met)PC-3 | |
| Colon ca. HCT-116 | 3.7 | Testis | 9.8 |
| Colon ca. CaCo-2 | 10.3 | Melanoma | 6.1 |
| | | Hs688(A).T | |
| Colon ca. | 9.2 | Melanoma* (met) | 6.0 |
| tissue (ODO3866) | | Hs688(B).T | |
| Colon ca. HCC-2998 | 5.3 | Melanoma UACC-62 | 1.8 |
| Gastric ca.* (liver met) | 34.9 | Melanoma M14 | 14.5 |
| NCI-N87 | | | |
| Bladder | 14.0 | Melanoma LOX | 0.9 |
| | | IMVI | |
| Trachea | 6.5 | Melanoma* (met) | 3.8 |
| | | SK-MEL-5 | |
| Kidney | 3.0 | Adipose | 12.8 |

TABLE BZD
Panel 2D

| | Rel. Exp. (%) | | Rel. Exp. (%) |
|---|---|---|---|
| | Ag3057, Run | | Ag3057, |
| Tissue Name | 163577596 | Tissue Name | Run 163577596 |
| Normal Colon | 81.8 | Kidney Margin | 1.1 |
| | | 8120608 | |
| CC Well to Mod Diff | 10.7 | Kidney Cancer | 8.1 |
| (ODO3866) | | 8120613 | |
| CC Margin (ODO3866) | 17.6 | Kidney Margin | 1.1 |
| | | 8120614 | |
| CC Gr.2 rectosigmoid | 11.2 | Kidney Cancer | 2.7 |
| (ODO3868) | | 9010320 | |
| CC Margin (ODO3868) | 4.8 | Kidney Margin | 2.8 |
| | | 9010321 | |
| CC Mod Diff (ODO3920) | 15.5 | Normal Uterus | 10.7 |
| CC Margin (ODO3920) | 20.2 | Uterus Cancer 064011 | 31.2 |
| CC Gr.2 ascend colon | 36.9 | Normal Thyroid | 19.1 |
| (ODO3921) | | | |
| CC Margin (ODO3921) | 9.7 | Thyroid Cancer | 7.6 |
| | | 064010 | |
| CC from Partial | 26.2 | Thyroid Cancer | 5.6 |
| Hepatectomy (ODO4309) | | A302152 | |
| Mets | | | |
| Liver Margin (ODO4309) | 13.1 | Thyroid Margin | 13.7 |
| | | A302153 | |
| Colon mets to lung | 2.9 | Normal Breast | 41.5 |
| (OD04451-01) | | | |
| Lung Margin (OD04451- | 6.0 | Breast Cancer | 7.5 |
| 02) | | (OD04566) | |
| Normal Prostate 6546-1 | 60.3 | Breast Cancer | 40.6 |
| | | (OD04590-01) | |
| Prostate Cancer | 20.2 | Breast Cancer Mets | 32.3 |
| (OD04410) | | (OD04590-03) | |
| Prostate Margin | 24.5 | Breast Cancer | 17.9 |
| (OD04410) | | Metastasis | |
| | | (OD04655-05) | |
| Prostate Cancer | 23.8 | Breast Cancer 064006 | 15.7 |
| (OD04720-01) | | | |
| Prostate Margin | 37.4 | Breast Cancer 1024 | 10.2 |
| (OD04720-02) | | | |
| Normal Lung 061010 | 42.6 | Breast Cancer | 6.5 |
| | | 9100266 | |
| Lung Met to Muscle | 38.7 | Breast Margin | 6.4 |
| (ODO4286) | | 9100265 | |
| Muscle Margin | 9.2 | Breast Cancer | 20.2 |
| (ODO4286) | | A209073 | |
| Lung Malignant Cancer | 20.7 | Breast Margin | 15.2 |
| (OD03126) | | A2090734 | |
| Lung Margin (OD03126) | 17.8 | Normal Liver | 7.4 |
| Lung Cancer (OD04404) | 36.9 | Liver Cancer 064003 | 7.2 |
| Lung Margin (OD04404) | 11.0 | Liver Cancer 1025 | 3.2 |
| Lung Cancer (OD04565) | 11.3 | Liver Cancer 1026 | 3.5 |
| Lung Margin (OD04565) | 10.6 | Liver Cancer 6004-T | 4.8 |
| Lung Cancer (OD04237- | 30.4 | Liver Tissue 6004-N | 4.8 |
| 01) | | | |
| Lung Margin (OD04237- | 21.0 | Liver Cancer 6005-T | 2.5 |
| 02) | | | |
| Ocular Mel Met to Liver | 11.7 | Liver Tissue 6005-N | 1.1 |
| (ODO4310) | | | |
| Liver Margin (ODO4310) | 13.5 | Normal Bladder | 32.1 |
| Melanoma Mets to Lung | 10.2 | Bladder Cancer 1023 | 5.6 |
| (OD04321) | | | |
| Lung Margin (OD04321) | 36.3 | Bladder Cancer | 49.3 |
| | | A302173 | |
| Normal Kidney | 100.0 | Bladder Cancer | 28.7 |
| | | (OD04718-01) | |
| Kidney Ca, Nuclear grade | 40.6 | Bladder Normal | 24.8 |
| 2 (OD04338) | | Adjacent (OD04718- | |
| | | 03) | |
| Kidney Margin | 16.8 | Normal Ovary | 6.5 |
| (OD04338) | | | |
| Kidney Ca Nuclear grade | 11.0 | Ovarian Cancer | 21.5 |
| 1/2 (OD04339) | | 064008 | |
| Kidney Margin | 21.9 | Ovarian Cancer | 35.8 |
| (OD04339) | | (OD04768-07) | |
| Kidney Ca, Clear cell | 66.4 | Ovary Margin | 5.4 |
| type (OD04340) | | (OD04768-08) | |
| Kidney Margin | 18.0 | Normal Stomach | 80.1 |
| (OD04340) | | | |
| Kidney Ca, Nuclear grade | 3.4 | Gastric Cancer | 2.2 |
| 3 (OD04348) | | 9060358 | |
| Kidney Margin | 10.2 | Stomach Margin | 12.3 |
| (OD04348) | | 9060359 | |
| Kidney Cancer | 12.3 | Gastric Cancer | 23.2 |
| (OD04622-01) | | 9060395 | |
| Kidney Margin | 1.2 | Stomach Margin | 14.6 |
| (OD04622-03) | | 9060394 | |
| Kidney Cancer | 26.1 | Gastric Cancer | 11.8 |
| (OD04450-01) | | 9060397 | |
| Kidney Margin | 21.5 | Stomach Margin | 5.8 |
| (OD04450-03) | | 9060396 | |
| Kidney Cancer 8120607 | 2.2 | Gastric Cancer | 55.5 |
| | | 064005 | |

TABLE BZE
Panel CNS_1

| | Rel. Exp. (%) Ag3057, | | Rel. Exp. (%) Ag3057, |
|---|---|---|---|
| Tissue Name | Run 171694175 | Tissue Name | Run 171694175 |
| BA4 Control | 7.9 | BA17 PSP | 21.3 |
| BA4 Control2 | 16.0 | BA17 PSP2 | 5.4 |
| BA4 | 2.9 | Sub Nigra Control | 58.6 |
| Alzheimer's2 | | | |
| BA4 Parkinson's | 32.8 | Sub Nigra Control2 | 25.9 |
| BA4 | 46.7 | Sub Nigra | 20.0 |
| Parkinson's2 | | Alzheimer's2 | |
| BA4 | 25.9 | Sub Nigra | 85.3 |
| Huntington's | | Parkinson's2 | |
| BA4 | 4.1 | Sub Nigra | 100.0 |
| Huntington's2 | | Huntington's | |
| BA4 PSP | 5.7 | Sub Nigra | 59.0 |
| | | Huntington's2 | |
| BA4 PSP2 | 36.9 | Sub Nigra PSP2 | 25.2 |
| BA4 Depression | 15.6 | Sub Nigra | 35.8 |
| | | Depression | |
| BA4 | 27.2 | Sub Nigra | 26.1 |
| Depression2 | | Depression2 | |
| BA7 Control | 12.9 | Glob Palladus | 54.3 |
| | | Control | |
| BA7 Control2 | 12.4 | Glob Palladus | 15.2 |
| | | Control2 | |
| BA7 | 5.2 | Glob Palladus | 17.4 |
| Alzheimer's2 | | Alzheimer's | |
| BA7 Parkinson's | 15.4 | Glob Palladus | 15.6 |
| | | Alzheimer's2 | |
| BA7 | 23.2 | Glob Palladus | 73.7 |
| Parkinson's2 | | Parkinson's | |
| BA7 | 34.9 | Glob Palladus | 15.8 |
| Huntington's | | Parkinson's2 | |
| BA7 | 56.3 | Glob Palladus PSP | 17.7 |
| Huntington's2 | | | |
| BA7 PSP | 29.1 | Glob Palladus PSP2 | 8.4 |
| BA7 PSP2 | 12.6 | Glob Palladus | 24.1 |
| | | Depression | |
| BA7 Depression | 12.2 | Temp Pole Control | 0.8 |
| BA9 Control | 4.4 | Temp Pole Control2 | 13.9 |
| BA9 Control2 | 36.1 | Temp Pole | 2.7 |
| | | Alzheimer's | |
| BA9 Alzheimer's | 3.8 | Temp Pole | 2.0 |
| | | Alzheimer's2 | |
| BA9 | 2.1 | Temp Pole | 15.9 |
| Alzheimer's2 | | Parkinson's | |
| BA9 Parkinson's | 17.2 | Temp Pole | 12.9 |
| | | Parkinson's2 | |
| BA9 | 20.7 | Temp Pole | 19.2 |
| Parkinson's2 | | Huntington's | |
| BA9 | 36.9 | Temp Pole PSP | 6.2 |
| Huntington's | | | |
| BA9 | 15.2 | Temp Pole PSP2 | 1.8 |
| Huntington's2 | | | |
| BA9 PSP | 27.4 | Temp Pole | 10.8 |
| | | Depression2 | |
| BA9 PSP2 | 14.0 | Cing Gyr Control | 35.6 |
| BA9 Depression | 7.5 | Cing Gyr Control2 | 15.2 |
| BA9 | 11.7 | Cing Gyr | 15.7 |
| Depression2 | | Alzheimer's | |
| BA17 Control | 20.7 | Cing Gyr | 10.7 |
| | | Alzheimer's2 | |
| BA17 Control2 | 15.3 | Cing Gyr Parkinson's | 47.0 |
| BA17 | 4.2 | Cing Gyr | 48.6 |
| Alzheimer's2 | | Parkinson's2 | |
| BA17 | 41.8 | Cing Gyr | 85.3 |
| Parkinson's | | Huntington's | |
| BA17 | 27.5 | Cing Gyr | 53.2 |
| Parkinson's2 | | Huntington's2 | |
| BA17 | 17.3 | Cing Gyr PSP | 80.1 |
| Huntington's | | | |
| BA17 | 27.0 | Cing Gyr PSP2 | 6.9 |
| Huntington's2 | | | |
| BA17 | 81.8 | Cing Gyr Depression | 18.6 |
| Depression | | | |
| BA17 | 51.4 | Cing Gyr | 69.7 |
| Depression2 | | Depression2 | |

CNS_neurodegeneration_v1.0 Summary: Ag3057 The NOV85 gene is found to be slightly but significantly (p=0.016) upregulated in the Alzheimer's disease (AD) temporal cortex. The temporal cortex is the region of the brain where neurons degenerate in the mid stages of AD. This increase in expression is not apparent in the occipital cortex, which does not experience neurodegeneration in AD. Since the upregulation of this gene appears to be neurodegeneration-specific both within an individual brain and between brains, this gene is an excellent small molecule target. Therefore, treatment with an antagonist may decrease the pathology seen in Alzheimer's disease.

Panel 1.3D Summary: Ag3057 Highest expression of the NOV85 gene is seen in the CNS. Please see CNS_Neurodegeneration for discussion of utility of this gene in the central nervous system.

Among tissues with metabolic function, this gene has low levels of expression in pancreas, adrenal, thyroid, pituitary, skeletal muscle and adipose. Therefore, modulation of this gene product may be a treatment for metabolic and endocrine diseases, including obesity and Types 1 and 2 diabetes.

In addition, this gene is expressed at low levels in the cancer cell lines in this panel. This difference in expression is particularly prominent in the CNS cancer cell lines when compared to the normal brain tissues. Thus, this gene could potentially be used as a diagnostic marker in CNS cancers.

Panel 2D Summary: Ag3057 The NOV85 gene is expressed at moderate to low levels in this panel. A higher level of expression is observed in lung, kidney, uterine, gastric and ovarian cancer when compared to the normal adjacent lung, kidney, uterine, gastric and ovarian tissues in this panel. Thus, this gene could be used as a diagnostic marker of cancer in these tissues. Futhermore, inhibition of the activity of this gene product using small molecule drugs may be useful for the treatment of cancer in these tissues

Panel 4D Summary: Ag3057 The amp plot indicates that there is a high probability of experimental failure. (Data not shown.)

Panel CNS_{—}1 Summary: Ag3057 These results confirm expression of the NOV85 gene in the brain. Please see CNS_Neurodegeneration for discussion of utility of this gene in the central nervous system.

NOV86: GTPase-Activating Protein

Expression of gene NOV86 was assessed using the primer-probe set Ag3058, described in Table CAA. Results of the RTQ-PCR runs are shown in Tables CAB, CAC and CAD.

TABLE CAA
Probe Name Ag3058

| | | | Start | |
|---|---|---|---|---|
| Primers | Sequences | Length | Position | SEQ ID NO: |
| Forward | 5′-agtacccgctgctgaacac-3′ | 19 | 534 | 1339 |
| Probe | TET-5′-accctcattgccaaggtcaaagcct-3′-TAMRA | 25 | 578 | 1340 |
| Reverse | 5′-tcattgttgctctcataatgga-3′ | 22 | 603 | 1341 |

TABLE CAB
Panel 1.3D

| | Rel. Exp. (%) Ag3058, | | Rel. Exp. (%) Ag3058, |
|---|---|---|---|
| Tissue Name | Run 165533238 | Tissue Name | Run 165533238 |
| Liver adenocarcinoma | 5.5 | Kidney (fetal) | 0.9 |
| Pancreas | 1.2 | Renal ca. 786-0 | 0.6 |
| Pancreatic ca. CAPAN 2 | 3.9 | Renal ca. A498 | 0.1 |
| Adrenal gland | 1.8 | Renal ca. RXF 393 | 1.0 |
| Thyroid | 1.7 | Renal ca. ACHN | 0.4 |
| Salivary gland | 6.4 | Renal ca. UO-31 | 15.7 |
| Pituitary gland | 1.1 | Renal ca. TK-10 | 0.1 |
| Brain (fetal) | 0.6 | Liver | 3.5 |
| Brain (whole) | 2.7 | Liver (fetal) | 4.9 |
| Brain (amygdala) | 3.0 | Liver ca. | 5.3 |
| | | (hepatoblast) HepG2 | |
| Brain (cerebellum) | 0.4 | Lung | 16.5 |
| Brain (hippocampus) | 2.4 | Lung (fetal) | 8.4 |
| Brain (substantia nigra) | 4.7 | Lung ca. (small cell) | 1.8 |
| | | LX-1 | |
| Brain (thalamus) | 2.7 | Lung ca. (small cell) | 1.0 |
| | | NCI-H69 | |
| Cerebral Cortex | 1.2 | Lung ca. (s.cell var.) | 1.3 |
| | | SHP-77 | |
| Spinal cord | 4.2 | Lung ca. (large | 0.9 |
| | | cell)NCI-H460 | |
| glio/astro U87-MG | 0.0 | Lung ca. (non-sm. | 0.1 |
| | | cell) A549 | |
| glio/astro U-118-MG | 0.2 | Lung ca. (non-s.cell) | 0.2 |
| | | NCI-H23 | |
| astrocytoma SW1783 | 0.1 | Lung ca. (non-s.cell) | 4.6 |
| | | HOP-62 | |
| neuro*; met SK-N-AS | 0.0 | Lung ca. (non-s.cl) | 0.0 |
| | | NCI-H522 | |
| astrocytoma SF-539 | 2.5 | Lung ca. (squam.) | 6.9 |
| | | SW 900 | |
| astrocytoma SNB-75 | 5.4 | Lung ca. (squam.) | 0.5 |
| | | NCI-H596 | |
| glioma SNB-19 | 0.7 | Mammary gland | 1.7 |
| glioma U251 | 1.1 | Breast ca.* (pl.ef) | 0.0 |
| | | MCF-7 | |
| glioma SF-295 | 0.3 | Breast ca.* (pl.ef) | 19.2 |
| | | MDA-MB-231 | |
| Heart (fetal) | 1.0 | Breast ca.* (pl.ef) | 2.4 |
| | | T47D | |
| Heart | 1.4 | Breast ca. BT-549 | 4.7 |
| Skeletal muscle (fetal) | 0.5 | Breast ca. MDA-N | 0.9 |
| Skeletal muscle | 1.0 | Ovary | 1.8 |
| Bone marrow | 32.1 | Ovarian ca. OVCAR-3 | 0.3 |
| Thymus | 29.3 | Ovarian ca. OVCAR-4 | 0.3 |
| Spleen | 46.3 | Ovarian ca. OVCAR-5 | 9.0 |
| Lymph node | 100.0 | Ovarian ca. OVCAR-8 | 0.7 |
| Colorectal | 1.4 | Ovarian ca. IGROV-1 | 0.7 |
| Stomach | 12.1 | Ovarian ca.* (ascites) | 0.2 |
| | | SK-OV-3 | |
| Small intestine | 16.6 | Uterus | 2.4 |
| Colon ca. SW480 | 2.3 | Placenta | 13.8 |
| Colon ca.* | 0.9 | Prostate | 0.9 |
| SW620 (SW480 met) | | | |
| Colon ca. HT29 | 1.2 | Prostate ca.* (bone | 3.1 |
| | | met)PC-3 | |
| Colon ca. HCT-116 | 2.9 | Testis | 0.2 |
| Colon ca. CaCo-2 | 3.5 | Melanoma | 0.1 |
| | | Hs688(A).T | |
| Colon ca. | 4.6 | Melanoma* (met) | 0.0 |
| tissue (ODO3866) | | Hs688(B).T | |
| Colon ca. HCC-2998 | 2.8 | Melanoma UACC-62 | 0.1 |
| Gastric ca.* (liver met) | 4.6 | Melanoma M14 | 7.2 |
| NCI-N87 | | | |
| Bladder | 1.9 | Melanoma LOX | 1.6 |
| | | IMVI | |
| Trachea | 8.1 | Melanoma* (met) | 0.1 |
| | | SK-MEL-5 | |
| Kidney | 0.3 | Adipose | 3.5 |

TABLE CAC
Panel 2D

| | Rel. Exp. (%) | | Rel. Exp. (%) |
|---|---|---|---|
| | Ag3058, Run | | Ag3058, |
| Tissue Name | 162569974 | Tissue Name | Run 162569974 |
| Normal Colon | 30.8 | Kidney Margin | 6.5 |
| | | 8120608 | |
| CC Well to Mod Diff | 6.8 | Kidney Cancer | 2.9 |
| (ODO3866) | | 8120613 | |
| CC Margin (ODO3866) | 6.7 | Kidney Margin | 6.3 |
| | | 8120614 | |
| CC Gr.2 rectosigmoid | 8.5 | Kidney Cancer | 35.4 |
| (ODO3868) | | 9010320 | |
| CC Margin (ODO3868) | 1.3 | Kidney Margin | 17.0 |
| | | 9010321 | |
| CC Mod Diff (ODO3920) | 9.9 | Normal Uterus | 2.4 |
| CC Margin (ODO3920) | 11.3 | Uterus Cancer 064011 | 10.2 |
| CC Gr.2 ascend colon | 14.7 | Normal Thyroid | 6.8 |
| (ODO3921) | | | |
| CC Margin (ODO3921) | 10.7 | Thyroid Cancer | 5.1 |
| | | 064010 | |
| CC from Partial | 18.7 | Thyroid Cancer | 6.8 |
| Hepatectomy (ODO4309) | | A302152 | |
| Mets | | | |
| Liver Margin (ODO4309) | 12.4 | Thyroid Margin | 7.9 |
| | | A302153 | |
| Colon mets to lung | 12.7 | Normal Breast | 17.7 |
| (OD04451-01) | | | |
| Lung Margin (OD04451- | 18.7 | Breast Cancer | 10.4 |
| 02) | | (OD04566) | |
| Normal Prostate 6546-1 | 13.8 | Breast Cancer | 18.2 |
| | | (OD04590-01) | |
| Prostate Cancer | 13.8 | Breast Cancer Mets | 59.9 |
| (OD04410) | | (OD04590-03) | |
| Prostate Margin | 11.7 | Breast Cancer | 52.5 |
| (OD04410) | | Metastasis | |
| | | (OD04655-05) | |
| Prostate Cancer | 7.3 | Breast Cancer 064006 | 21.3 |
| (OD04720-01) | | | |
| Prostate Margin | 19.2 | Breast Cancer 1024 | 9.7 |
| (OD04720-02) | | | |
| Normal Lung 061010 | 100.0 | Breast Cancer | 12.9 |
| | | 9100266 | |
| Lung Met to Muscle | 29.5 | Breast Margin | 14.1 |
| (ODO4286) | | 9100265 | |
| Muscle Margin | 8.2 | Breast Cancer | 11.6 |
| (ODO4286) | | A209073 | |
| Lung Malignant Cancer | 31.6 | Breast Margin | 6.6 |
| (OD03126) | | A2090734 | |
| Lung Margin (OD03126) | 48.6 | Normal Liver | 7.2 |
| Lung Cancer (OD04404) | 16.4 | Liver Cancer 064003 | 5.4 |
| Lung Margin (OD04404) | 17.1 | Liver Cancer 1025 | 6.8 |
| Lung Cancer (OD04565) | 6.7 | Liver Cancer 1026 | 13.2 |
| Lung Margin (OD04565) | 20.3 | Liver Cancer 6004-T | 7.2 |
| Lung Cancer (OD04237- | 24.0 | Liver Tissue 6004-N | 6.7 |
| 01) | | | |
| Lung Margin (OD04237- | 43.2 | Liver Cancer 6005-T | 14.7 |
| 02) | | | |
| Ocular Mel Met to Liver | 3.4 | Liver Tissue 6005-N | 4.5 |
| (ODO4310) | | | |
| Liver Margin (ODO4310) | 8.2 | Normal Bladder | 20.2 |
| Melanoma Mets to Lung | 6.0 | Bladder Cancer 1023 | 9.2 |
| (OD04321) | | | |
| Lung Margin (OD04321) | 69.3 | Bladder Cancer | 9.6 |
| | | A302173 | |
| Normal Kidney | 13.0 | Bladder Cancer | 38.2 |
| | | (OD04718-01) | |
| Kidney Ca, Nuclear grade | 22.5 | Bladder Normal | 14.9 |
| 2 (OD04338) | | Adjacent (OD04718- | |
| | | 03) | |
| Kidney Margin | 11.1 | Normal Ovary | 5.8 |
| (OD04338) | | | |
| Kidney Ca Nuclear grade | 20.4 | Ovarian Cancer | 17.4 |
| 1/2 (OD04339) | | 064008 | |
| Kidney Margin | 6.5 | Ovarian Cancer | 3.7 |
| (OD04339) | | (OD04768-07) | |
| Kidney Ca, Clear cell | 28.5 | Ovary Margin | 5.2 |
| type (OD04340) | | (OD04768-08) | |
| Kidney Margin | 13.7 | Normal Stomach | 14.8 |
| (OD04340) | | | |
| Kidney Ca, Nuclear grade | 9.7 | Gastric Cancer | 8.0 |
| 3 (OD04348) | | 9060358 | |
| Kidney Margin | 11.3 | Stomach Margin | 17.4 |
| (OD04348) | | 9060359 | |
| Kidney Cancer | 27.9 | Gastric Cancer | 17.7 |
| (OD04622-01) | | 9060395 | |
| Kidney Margin | 2.6 | Stomach Margin | 26.8 |
| (OD04622-03) | | 9060394 | |
| Kidney Cancer | 2.3 | Gastric Cancer | 20.2 |
| (OD04450-01) | | 9060397 | |
| Kidney Margin | 5.3 | Stomach Margin | 12.9 |
| (OD04450-03) | | 9060396 | |
| Kidney Cancer 8120607 | 7.4 | Gastric Cancer | 29.7 |
| | | 064005 | |

TABLE CAD
Panel 4D

| | Rel. Exp. (%) | | Rel. Exp. (%) |
|---|---|---|---|
| | Ag3058, Run | | Ag3058, Run |
| Tissue Name | 162562989 | Tissue Name | 162562989 |
| Secondary Th1 act | 5.9 | HUVEC IL-1beta | 21.9 |
| Secondary Th2 act | 6.2 | HUVEC IFN gamma | 16.8 |
| Secondary Tr1 act | 9.5 | HUVEC TNF alpha + IFN | 20.6 |
| | | gamma | |
| Secondary Th1 rest | 1.0 | HUVEC TNF alpha + IL4 | 28.1 |
| Secondary Th2 rest | 1.2 | HUVEC IL-11 | 12.3 |
| Secondary Tr1 rest | 1.4 | Lung Microvascular EC | 28.3 |
| | | none | |
| Primary Th1 act | 8.7 | Lung Microvascular EC | 46.7 |
| | | TNF alpha + IL-1beta | |
| Primary Th2 act | 3.5 | Microvascular Dermal EC | 38.7 |
| | | none | |
| Primary Tr1 act | 8.3 | Microsvascular Dermal EC | 24.7 |
| | | TNF alpha + IL-1beta | |
| Primary Th1 rest | 7.6 | Bronchial epithelium | 9.2 |
| | | TNF alpha + IL1beta | |
| Primary Th2 rest | 3.1 | Small airway epithelium | 13.8 |
| | | none | |
| Primary Tr1 rest | 4.8 | Small airway epithelium | 100.0 |
| | | TNF alpha + IL-1beta | |
| CD45RA CD4 | 13.7 | Coronery artery SMC rest | 22.2 |
| lymphocyte act | | | |
| CD45RO CD4 | 7.3 | Coronery artery SMC | 11.3 |
| lymphocyte act | | TNF alpha + IL-1beta | |
| CD8 lymphocyte act | 2.3 | Astrocytes rest | 14.7 |
| Secondary CD8 | 3.4 | Astrocytes TNF alpha + | 12.0 |
| lymphocyte rest | | IL-1beta | |
| Secondary CD8 | 4.4 | KU-812 (Basophil) rest | 12.2 |
| lymphocyte act | | | |
| CD4 lymphocyte none | 1.5 | KU-812 (Basophil) | 46.7 |
| | | PMA/ionomycin | |
| 2ry Th1/Th2/Tr1_anti- | 1.3 | CCD1106 (Keratinocytes) | 24.0 |
| CD95 CH11 | | none | |
| LAK cells rest | 15.3 | CCD1106 (Keratinocytes) | 1.6 |
| | | TNF alpha + IL-1beta | |
| LAK cells IL-2 | 4.5 | Liver cirrhosis | 3.7 |
| LAK cells IL-2 + IL-12 | 4.5 | Lupus kidney | 3.9 |
| LAK cells IL-2 + IFN | 8.1 | NCI-H292 none | 46.3 |
| gamma | | | |
| LAK cells IL-2 + IL-18 | 7.9 | NCI-H292 IL-4 | 57.4 |
| LAK cells | 10.2 | NCI-H292 IL-9 | 63.3 |
| PMA/ionomycin | | | |
| NK Cells IL-2 rest | 2.0 | NCI-H292 IL-13 | 28.1 |
| Two Way MLR 3 day | 2.6 | NCI-H292 IFN gamma | 25.9 |
| Two Way MLR 5 day | 3.1 | HPAEC none | 14.8 |
| Two Way MLR 7 day | 2.3 | HPAEC TNF alpha + IL-1 | 19.9 |
| | | beta | |
| PBMC rest | 2.4 | Lung fibroblast none | 29.1 |
| PBMC PWM | 12.3 | Lung fibroblast TNF alpha + | 13.3 |
| | | IL-1beta | |
| PBMC PHA-L | 5.1 | Lung fibroblast IL-4 | 39.8 |
| Ramos (B cell) none | 0.0 | Lung fibroblast IL-9 | 24.5 |
| Ramos (B cell) | 0.0 | Lung fibroblast IL-13 | 19.1 |
| ionomycin | | | |
| B lymphocytes PWM | 32.3 | Lung fibroblast IFN | 45.7 |
| | | gamma | |
| B lymphocytes CD40L | 4.1 | Dermal fibroblast | 57.4 |
| and IL-4 | | CCD1070 rest | |
| EOL-1 dbcAMP | 0.7 | Dermal fibroblast | 81.8 |
| | | CCD1070 TNF alpha | |
| EOL-1 dbcAMP | 6.7 | Dermal fibroblast | 25.0 |
| PMA/ionomycin | | CCD1070 IL-1beta | |
| Dendritic cells none | 12.8 | Dermal fibroblast IFN | 51.1 |
| | | gamma | |
| Dendritic cells LPS | 11.8 | Dermal fibroblast IL-4 | 52.9 |
| Dendritic cells anti- | 13.8 | IBD Colitis 2 | 0.7 |
| CD40 | | | |
| Monocytes rest | 6.2 | IBD Crohn's | 3.4 |
| Monocytes LPS | 6.5 | Colon | 22.5 |
| Macrophages rest | 18.3 | Lung | 19.2 |
| Macrophages LPS | 10.0 | Thymus | 24.7 |
| HUVEC none | 33.7 | Kidney | 13.0 |
| HUVEC starved | 49.0 | | |

Panel 1.3D Summary: Ag3058 Highest expression of the NOV86 gene, a GTPase-activating protein homolog, is seen in the lymph node (CT=27.8). Among tissues with metabolic function, this gene has low levels of expression in pancreas, adrenal, thyroid, pituitary, heart, skeletal muscle, liver and adipose. Rab GTPases are integral to vesicular transport in the secretory and endocytic pathways. Therefore, therapeutic modulation of this gene product may be a treatment for metabolic and endocrine diseases, including obesity and Types 1 and 2 diabetes.

This GTPase activating enzyme like molecule is also expressed at low levels in the CNS. Thus, it may be useful in treating diseases of the nervous system, stroke or CNS trauma.

In addition, this gene is expressed at low levels in the cancer cell lines in this panel. low/undetectable (CTs>35) across all of the samples on this panel (data not shown).

Panel 2D Summary: Ag3058 The NOV86 gene is expressed at low levels in this panel. There is higher expression in kidney, breast, liver and bladder cancer samples compared to the adjacent normal tissue. Conversely, there is lower expression in lung cancer samples compared to the adjacent normal tissue. Thus, the expression of this gene could be used as a diagnostic marker for kidney, breast, liver, bladder and lung cancers. Furthermore, modulation of expression of this gene may also be used for therapy of these cancers.

Panel 4D Summary: Ag3058 The NOV86 gene is expressed at high to moderate levels in a wide range of cell types of significance in the immune response and tissue response in health and disease, with the highest expression being detected in TNF alpha plus IL-1 beta treated small airway epithelial cells (CT=28.03). Therefore, targeting of this gene product with a small molecule drug or antibody therapeutic may modulate the functions of cells of the immune system as well as resident tissue cells and lead to improvement of the symptoms of patients suffering from autoimmune and inflammatory diseases such as COPD, emphysema, asthma, allergies, inflammatory bowel disease, lupus erythematosus, and arthritis, including osteoarthritis and rheumatoid arthritis.

NOV87a and NOV87b

Expression of gene NOV87a and full length clone NOV87b was assessed using the primer-probe set Ag3040, described in Table CBA.

TABLE CBA

Probe Name Ag3040

| | | | Start | |
|---|---|---|---|---|
| Primers | Sequences | Length | Position | SEQ ID NO: |
| Forward | 5′-gagccctgaagctcttctttc-3′ | 21 | 617 | 1342 |
| Probe | TET-5′-cttctcgcacttccgccagttcatt-3′-TAMRA | 25 | 663 | 1343 |
| Reverse | 5′-cctggtcctgctcactgat-3′ | 19 | 694 | 1344 |

CNS_neurodegeneration_v1.0 Summary: Ag3040 Expression of this gene is low/undetectable (CTs>35) across all of the samples on this panel (data not shown).

Panel 1.3D Summary: Ag3040 Expression of this gene is low/undetectable (CTs>35) across all of the samples on this panel (data not shown).

Panel 4D Summary: Ag3040 Expression of this gene is low/undetectable (CTs>35) across all of the samples on this panel (data not shown).

NOV88

Expression of gene NOV88 was assessed using the primer-probe set Ag2923, described in Table CCA.

TABLE CCA
Probe Name Ag2923

| | | | Start | |
|---|---|---|---|---|
| Primers | Sequences | Length | Position | SEQ ID NO: |
| Forward | 5′-agtcaacagatttggccacat-3′ | 21 | 45 | 1345 |
| Probe | TET-5′-tcaccagggctgcttttaactctggt-3′-TAMRA | 26 | 77 | 1346 |
| Reverse | 5′-cagtgaaggggtcactgatg-3′ | 20 | 120 | 1347 |

CNS_neurodegeneration_v1.0 Summary: Ag2923 Expression of this gene is low/undetectable (CTs>34.5) across all of the samples on this panel (data not shown).

Panel 1.3D Summary: Ag2923 Expression of this gene is low/undetectable (CTs>34.5) across all of the samples on this panel (data not shown).

Panel 2D Summary: Ag2923 Expression of this gene is low/undetectable (CTs>35) across all of the samples on this panel (data not shown).

Panel 4D Summary: Ag2923 Expression of this gene is low/undetectable (CTs>34.5) across all of the samples on this panel (data not shown).

NOV89

Expression of gene NOV89 was assessed using the primer-probe set Ag2924, described in Table CDA.

TABLE CDA
Probe Name Ag2924

| | | | Start | |
|---|---|---|---|---|
| Primers | Sequences | Length | Position | SEQ ID NO: |
| Forward | 5′-gaacgggaagcttgttatcaat-3′ | 122 | 1231 | 1348 |
| Probe | TET-5′-agatctcaccaaaatcaaatggggca-3′-TAMRA | 26 | 282 | 1349 |
| Reverse | 5′-atgatgtactcagtgccagcat-3′ | 22 | 308 | 1350 |

CNS_neurodegeneration_v1.0 Summary: Ag2924 Expression of this gene is low/undetectable (CTs>35) across all of the samples on this panel (data not shown).

Panel 1.3D Summary: Ag2924 Expression of this gene is low/undetectable (CTs>35) across all of the samples on this panel (data not shown).

Panel 4D Summary: Ag2924 Expression of this gene is low/undetectable (CTs>35) across all of the samples on this panel (data not shown).

Panel 5 Islet Summary: Ag2924 Run 242285280 Expression of this gene is low/undetectable (CTs>35) across all of the samples on this panel (data not shown). Run 243564308 The amp plot indicates that there were experimental difficulties with this run.

NOV90

Expression of gene NOV90 was assessed using the primer-probe set Ag3045, described in Table CEA. Results of the RTQ-PCR runs are shown in Tables CEB, CEC, CED, CEE, CEF and CEG.

TABLE CEA
Probe Name Ag3045

| | | | Start | |
|---|---|---|---|---|
| Primers | Sequences | Length | Position | SEQ ID NO: |
| Forward | 5′-caccggatcatatgaaatcaat-3′ | 22 | 605 | 1351 |
| Probe | TET-5′-tgtaattgaccctgttcctgcaccag-3′-TAMRA | 26 | 639 | 1352 |
| Reverse | 5′-ccaccatcaacatttgaatca-3′ | 21 | 1669 | 1353 |

TABLE CEB
CNS_neurodegeneration_v1.0

| | Rel. Exp. (%) Ag3045, | | Rel. Exp. (%) Ag3045, |
|---|---|---|---|
| Tissue Name | Run 211012233 | Tissue Name | Run 211012233 |
| AD 1 Hippo | 0.8 | Control (Path) 3 | 0.2 |
| | | Temporal Ctx | |
| AD 2 Hippo | 0.1 | Control (Path) 4 | 4.4 |
| | | Temporal Ctx | |
| AD 3 Hippo | 0.5 | AD 1 Occipital Ctx | 2.1 |
| AD 4 Hippo | 0.3 | AD 2 Occipital Ctx | 0.0 |
| | | (Missing) | |
| AD 5 hippo | 8.5 | AD 3 Occipital Ctx | 0.3 |
| AD 6 Hippo | 0.3 | AD 4 Occipital Ctx | 2.0 |
| Control 2 Hippo | 0.5 | AD 5 Occipital Ctx | 0.8 |
| Control 4 Hippo | 0.5 | AD 6 Occipital Ctx | 1.1 |
| Control (Path) 3 | 0.4 | Control 1 Occipital | 0.0 |
| Hippo | | Ctx | |
| AD 1 Temporal Ctx | 1.4 | Control 2 Occipital | 2.2 |
| | | Ctx | |
| AD 2 Temporal Ctx | 0.0 | Control 3 Occipital | 2.1 |
| | | Ctx | |
| AD 3 Temporal Ctx | 0.7 | Control 4 Occipital | 0.2 |
| | | Ctx | |
| AD 4 Temporal Ctx | 2.7 | Control (Path) 1 | 4.1 |
| | | Occipital Ctx | |
| AD 5 Inf Temporal | 100.0 | Control (Path) 2 | 2.4 |
| Ctx | | Occipital Ctx | |
| AD 5 SupTemporal | 4.4 | Control (Path) 3 | 0.0 |
| Ctx | | Occipital Ctx | |
| AD 6 Inf Temporal | 0.9 | Control (Path) 4 | 2.7 |
| Ctx | | Occipital Ctx | |
| AD 6 Sup Temporal | 0.9 | Control 1 Parietal | 0.0 |
| Ctx | | Ctx | |
| Control 1 Temporal | 0.0 | Control 2 Parietal | 4.1 |
| Ctx | | Ctx | |
| Control 2 Temporal | 0.7 | Control 3 Parietal | 1.5 |
| Ctx | | Ctx | |
| Control 3 Temporal | 1.5 | Control (Path) 1 | 2.6 |
| Ctx | | Parietal Ctx | |
| Control 4 Temporal | 0.6 | Control (Path) 2 | 2.6 |
| Ctx | | Parietal Ctx | |
| Control (Path) 1 | 3.1 | Control (Path) 3 | 0.2 |
| Temporal Ctx | | Parietal Ctx | |
| Control (Path) 2 | 3.6 | Control (Path) 4 | 3.4 |
| Temporal Ctx | | Parietal Ctx | |

TABLE CEC
Panel 1.3D

| | Rel. Exp. (%) Ag3045, | | Rel. Exp. (%) Ag3045, |
|---|---|---|---|
| Tissue Name | Run 165519994 | Tissue Name | Run 165519994 |
| Liver adenocarcinoma | 10.3 | Kidney (fetal) | 1.9 |
| Pancreas | 3.7 | Renal ca. 786-0 | 4.8 |
| Pancreatic ca. CAPAN 2 | 3.5 | Renal ca. A498 | 16.2 |
| Adrenal gland | 7.5 | Renal ca. RXF 393 | 6.6 |
| Thyroid | 2.2 | Renal ca. ACHN | 1.0 |
| Salivary gland | 8.6 | Renal ca. UO-31 | 6.7 |
| Pituitary gland | 28.7 | Renal ca. TK-10 | 1.1 |
| Brain (fetal) | 20.3 | Liver | 4.4 |
| Brain (whole) | 100.0 | Liver (fetal) | 1.9 |
| Brain (amygdala) | 8.4 | Liver ca. | 3.6 |
| | | (hepatoblast) HepG2 | |
| Brain (cerebellum) | 50.7 | Lung | 4.1 |
| Brain (hippocampus) | 36.6 | Lung (fetal) | 2.3 |
| Brain (substantia nigra) | 12.1 | Lung ca. (small cell) | 0.8 |
| | | LX-1 | |
| Brain (thalamus) | 29.7 | Lung ca. (small cell) | 2.5 |
| | | NCI-H69 | |
| Cerebral Cortex | 4.2 | Lung ca. (s.cell var.) | 0.6 |
| | | SHP-77 | |
| Spinal cord | 10.4 | Lung ca. (large | 2.1 |
| | | cell)NCI-H460 | |
| glio/astro U87-MG | 3.2 | Lung ca. (non-sm. | 0.0 |
| | | cell) A549 | |
| glio/astro U-118-MG | 6.6 | Lung ca. (non-s.cell) | 3.2 |
| | | NCI-H23 | |
| astrocytoma SW1783 | 2.4 | Lung ca. (non-s.cell) | 4.5 |
| | | HOP-62 | |
| neuro*; met SK-N-AS | 3.5 | Lung ca. (non-s.cl) | 0.6 |
| | | NCI-H522 | |
| astrocytoma SF-539 | 35.4 | Lung ca. (squam.) | 16.4 |
| | | SW 900 | |
| astrocytoma SNB-75 | 14.0 | Lung ca. (squam.) | 2.6 |
| | | NCI-H596 | |
| glioma SNB-19 | 37.9 | Mammary gland | 2.9 |
| glioma U251 | 44.4 | Breast ca.* (pl.ef) | 4.8 |
| | | MCF-7 | |
| glioma SF-295 | 0.4 | Breast ca.* (pl.ef) | 7.1 |
| | | MDA-MB-231 | |
| Heart (fetal) | 0.4 | Breast ca.* (pl.ef) | 8.5 |
| | | T47D | |
| Heart | 9.5 | Breast ca. BT-549 | 3.8 |
| skeletal muscle (fetal) | 1.0 | Breast ca. MDA-N | 1.3 |
| Skeletal muscle | 5.4 | Ovary | 1.3 |
| Bone marrow | 9.9 | Ovarian ca. OVCAR-3 | 2.8 |
| Thymus | 8.8 | Ovarian ca. OVCAR-4 | 0.9 |
| Spleen | 9.4 | Ovarian ca. OVCAR-5 | 3.6 |
| Lymph node | 21.8 | Ovarian ca. OVCAR-8 | 20.2 |
| Colorectal | 3.3 | Ovarian ca. IGROV-1 | 0.6 |
| Stomach | 6.3 | Ovarian ca.* (ascites) | 11.4 |
| | | SK-OV-3 | |
| Small intestine | 8.0 | Uterus | 18.8 |
| Colon ca. SW480 | 4.1 | Placenta | 7.0 |
| Colon ca.* | 2.1 | Prostate | 15.7 |
| SW620 (SW480 met) | | | |
| Colon ca. HT29 | 0.5 | Prostate ca.* (bone | 2.8 |
| | | met)PC-3 | |
| Colon ca. HCT-116 | 2.5 | Testis | 11.4 |
| Colon ca. CaCo-2 | 1.9 | Melanoma | 0.4 |
| | | Hs688(A).T | |
| Colon ca. | 2.4 | Melanoma* (met) | 0.4 |
| tissue (ODO3866) | | Hs688(B).T | |
| Colon ca. HCC-2998 | 2.4 | Melanoma UACC-62 | 3.1 |
| Gastric ca.* (liver met) | 3.3 | Melanoma M14 | 7.9 |
| NCI-N87 | | | |
| Bladder | 11.3 | Melanoma LOX | 0.0 |
| | | IMVI | |
| Trachea | 1.7 | Melanoma* (met) | 1.8 |
| | | SK-MEL-5 | |
| Kidney | 10.2 | Adipose | 1.4 |

TABLE CED
Panel 2D

| | Rel. Exp. (%) | | Rel. Exp. (%) |
|---|---|---|---|
| | Ag3045, Run | | Ag3045, |
| Tissue Name | 163577595 | Tissue Name | Run 163577595 |
| Normal Colon | 30.4 | Kidney Margin | 1.1 |
| | | 8120608 | |
| CC Well to Mod Diff | 2.9 | Kidney Cancer | 2.5 |
| (ODO3866) | | 8120613 | |
| CC Margin (ODO3866) | 1.1 | Kidney Margin | 0.2 |
| | | 8120614 | |
| CC Gr.2 rectosigmoid | 2.7 | Kidney Cancer | 2.9 |
| (ODO3868) | | 9010320 | |
| CC Margin (ODO3868) | 4.1 | Kidney Margin | 4.0 |
| | | 9010321 | |
| CC Mod Diff (ODO3920) | 1.9 | Normal Uterus | 2.1 |
| CC Margin (ODO3920) | 3.9 | Uterus Cancer 064011 | 22.2 |
| CC Gr.2 ascend colon | 2.7 | Normal Thyroid | 7.1 |
| (ODO3921) | | | |
| CC Margin (ODO3921) | 2.6 | Thyroid Cancer | 2.0 |
| | | 064010 | |
| CC from Partial | 3.6 | Thyroid Cancer | 2.0 |
| Hepatectomy(ODO4309) | | A302152 | |
| Mets | | | |
| Liver Margin (ODO4309) | 3.6 | Thyroid Margin | 20.9 |
| | | A302153 | |
| Colon mets to lung | 1.8 | Normal Breast | 9.6 |
| (OD04451-01) | | | |
| Lung Margin (OD04451- | 2.5 | Breast Cancer | 5.3 |
| 02) | | (OD04566) | |
| Normal Prostate 6546-1 | 100.0 | Breast Cancer | 55.5 |
| | | (OD04590-01) | |
| Prostate Cancer | 39.0 | Breast Cancer Mets | 92.7 |
| (OD04410) | | (OD04590-03) | |
| Prostate Margin | 17.3 | Breast Cancer | 31.9 |
| (OD04410) | | Metastasis | |
| | | (OD04655-05) | |
| Prostate Cancer | 15.1 | Breast Cancer 064006 | 21.8 |
| (OD04720-01) | | | |
| Prostate Margin | 37.1 | Breast Cancer 1024 | 6.5 |
| (OD04720-02) | | | |
| Normal Lung 061010 | 47.0 | Breast Cancer | 8.4 |
| | | 9100266 | |
| Lung Met to Muscle | 4.4 | Breast Margin | 4.0 |
| (ODO4286) | | 9100265 | |
| Muscle Margin | 10.3 | Breast Cancer | 12.2 |
| (ODO4286) | | A209073 | |
| Lung Malignant Cancer | 8.1 | Breast Margin | 2.2 |
| (OD03126) | | A2090734 | |
| Lung Margin (OD03126) | 8.8 | Normal Liver | 1.0 |
| Lung Cancer (OD04404) | 47.0 | Liver Cancer 064003 | 0.8 |
| Lung Margin (OD04404) | 11.0 | Liver Cancer 1025 | 0.7 |
| Lung Cancer (OD04565) | 6.9 | Liver Cancer 1026 | 0.6 |
| Lung Margin (OD04565) | 1.7 | Liver Cancer 6004-T | 3.1 |
| Lung Cancer (OD04237- | 12.2 | Liver Tissue 6004-N | 1.7 |
| 01) | | | |
| Lung Margin (OD04237- | 5.4 | Liver Cancer 6005-T | 0.8 |
| 02) | | | |
| Ocular Mel Met to Liver | 5.1 | Liver Tissue 6005-N | 0.8 |
| (ODO4310) | | | |
| Liver Margin (ODO4310) | 1.0 | Normal Bladder | 12.5 |
| Melanoma Mets to Lung | 3.3 | Bladder Cancer 1023 | 1.4 |
| (OD04321) | | | |
| Lung Margin (OD04321) | 5.7 | Bladder Cancer | 6.7 |
| | | A302173 | |
| Normal Kidney | 7.2 | Bladder Cancer | 16.4 |
| | | (OD04718-01) | |
| Kidney Ca, Nuclear grade | 8.4 | Bladder Normal | 23.3 |
| 2 (OD04338) | | Adjacent (OD04718- | |
| | | 03) | |
| Kidney Margin | 2.9 | Normal Ovary | 2.0 |
| (OD04338) | | | |
| Kidney Ca Nuclear grade | 0.8 | Ovarian Cancer | 5.6 |
| 1/2 (OD04339) | | 064008 | |
| Kidney Margin | 1.8 | Ovarian Cancer | 2.4 |
| (OD04339) | | (OD04768-07) | |
| Kidney Ca, Clear cell | 2.1 | Ovary Margin | 2.4 |
| type (OD04340) | | (OD04768-08) | |
| Kidney Margin | 4.2 | Normal Stomach | 1.0 |
| (OD04340) | | | |
| Kidney Ca, Nuclear grade | 1.6 | Gastric Cancer | 0.5 |
| 3 (OD04348) | | 9060358 | |
| Kidney Margin | 10.4 | Stomach Margin | 2.4 |
| (OD04348) | | 9060359 | |
| Kidney Cancer | 1.3 | Gastric Cancer | 4.5 |
| (OD04622-01) | | 9060395 | |
| Kidney Margin | 1.1 | Stomach Margin | 1.9 |
| (OD04622-03) | | 9060394 | |
| Kidney Cancer | 9.0 | Gastric Cancer | 7.7 |
| (OD04450-01) | | 9060397 | |
| Kidney Margin | 14.0 | Stomach Margin | 0.3 |
| (OD04450-03) | | 9060396 | |
| Kidney Cancer 8120607 | 0.9 | Gastric Cancer | 8.3 |
| | | 064005 | |

TABLE CEE
Panel 3D

| | Rel. Exp. (%) | | Rel. Exp. (%) |
|---|---|---|---|
| | Ag3045, Run | | Ag3045, Run |
| Tissue Name | 164886427 | Tissue Name | 164886427 |
| Daoy-Medulloblastoma | 3.4 | Ca Ski-Cervical epidermoid | 6.6 |
| | | carcinoma (metastasis) | |
| TE671-Medulloblastoma | 4.4 | ES-2-Ovarian clear cell | 2.1 |
| | | carcinoma | |
| D283 Med- | 4.2 | Ramos-Stimulated with | 6.0 |
| Medulloblastoma | | PMA/ionomycin 6 h | |
| PFSK-1-Primitive | 1.7 | Ramos-Stimulated with | 1.7 |
| Neuroectodermal | | PMA/ionomycin 14 h | |
| XF-498-CNS | 7.1 | MEG-01-Chronic | 4.2 |
| | | myelogenous leukemia | |
| | | (megokaryoblast) | |
| SNB-78-Glioma | 2.2 | Raji-Burkitt's lymphoma | 2.0 |
| SF-268-Glioblastoma | 0.8 | Daudi-Burkitt's lymphoma | 3.4 |
| T98G-Glioblastoma | 1.3 | U266-B-cell plasmacytoma | 4.9 |
| SK-N-SH- | 3.1 | CA46-Burkitt's lymphoma | 0.5 |
| Neuroblastoma | | | |
| (metastasis) | | | |
| SF-295-Glioblastoma | 2.0 | RL-non-Hodgkin's B-cell | 2.1 |
| | | lymphoma | |
| Cerebellum | 100.0 | JM1-pre-B-cell lymphoma | 5.8 |
| Cerebellum | 1.4 | Jurkat-T cell leukemia | 3.5 |
| NCI-H292- | 23.0 | TF-1-Erythroleukemia | 8.3 |
| Mucoepidermoid lung | | | |
| carcinoma | | | |
| DMS-114-Small cell | 8.4 | HUT 78-T-cell lymphoma | 3.4 |
| lung cancer | | | |
| DMS-79-Small cell lung | 26.6 | U937-Histiocytic lymphoma | 1.2 |
| cancer | | | |
| NCI-H146-Small cell | 5.0 | KU-812-Myelogenous | 2.6 |
| lung cancer | | leukemia | |
| NCI-H526-Small cell | 30.6 | 769-P-Clear cell renal | 1.4 |
| lung cancer | | carcinoma | |
| NCI-N417-Small cell | 1.3 | Caki-2-Clear cell renal | 3.3 |
| lung cancer | | carcinoma | |
| NCI-H82-Small cell | 4.2 | SW 839-Clear cell renal | 1.6 |
| lung cancer | | carcinoma | |
| NCI-H157-Squamous | 2.6 | G401-Wilms' tumor | 2.0 |
| cell lung cancer | | | |
| (metastasis) | | | |
| NCI-H1155-Large cell | 50.0 | Hs766T-Pancreatic | 5.3 |
| lung cancer | | carcinoma (LN metastasis) | |
| NCI-H1299-Large cell | 10.5 | CAPAN-1-Pancreatic | 2.6 |
| lung cancer | | adenocarcinoma (liver | |
| | | metastasis) | |
| NCI-H727-Lung | 2.6 | SU86.86-Pancreatic | 6.5 |
| carcinoid | | carcinoma (liver metastasis) | |
| NCI-UMC-11-Lung | 8.5 | BxPC-3-Pancreatic | 1.5 |
| carcinoid | | adenocarcinoma | |
| LX-1-Small cell lung | 2.0 | HPAC-Pancreatic | 2.8 |
| cancer | | adenocarcinoma | |
| Colo-205-Colon cancer | 3.0 | MIA PaCa-2-Pancreatic | 1.9 |
| | | carcinoma | |
| KM12-Colon cancer | 2.6 | CFPAC-1-Pancreatic ductal | 6.9 |
| | | adenocarcinoma | |
| KM20L2-Colon cancer | 0.8 | PANC-1-Pancreatic | 7.5 |
| | | epithelioid ductal carcinoma | |
| NCI-H716-Colon cancer | 3.7 | T24-Bladder carcinma | 2.3 |
| | | (transitional cell) | |
| SW-48-Colon | 2.6 | 5637-Bladder carcinoma | 4.0 |
| adenocarcinoma | | | |
| SW1116-Colon | 1.0 | HT-1197-Bladder carcinoma | 3.8 |
| adenocarcinoma | | | |
| LS 174T-Colon | 3.2 | UM-UC-3-Bladder carcinma | 1.0 |
| adenocarcinoma | | (transitional cell) | |
| SW-948-Colon | 0.0 | A204-Rhabdomyosarcoma | 1.2 |
| adenocarcinoma | | | |
| SW-480-Colon | 1.1 | HT-1080-Fibrosarcoma | 6.8 |
| adenocarcinoma | | | |
| NCI-SNU-5-Gastric | 3.2 | MG-63-Osteosarcoma | 1.3 |
| carcinoma | | | |
| KATO III-Gastric | 3.9 | SK-LMS-1-Leiomyosarcoma | 6.3 |
| carcinoma | | (vulva) | |
| NCI-SNU-16-Gastric | 1.6 | SJRH30-Rhabdomyosarcoma | 2.6 |
| carcinoma | | (met to bone marrow) | |
| NCI-SNU-1-Gastric | 3.2 | A431-Epidermoid carcinoma | 5.1 |
| carcinoma | | | |
| RF-1-Gastric | 4.2 | WM266-4-Melanoma | 4.5 |
| adenocarcinoma | | | |
| RF-48-Gastric | 4.5 | DU 145-Prostate carcinoma | 1.0 |
| adenocarcinoma | | (brain metastasis) | |
| MKN-45-Gastric | 4.7 | MDA-MB-468-Breast | 2.3 |
| carcinoma | | adenocarcinoma | |
| NCI-N87-Gastric | 0.9 | SCC-4-Squamous cell | 1.4 |
| carcinoma | | carcinoma of tongue | |
| OVCAR-5-Ovarian | 1.4 | SCC-9-Squamous cell | 0.3 |
| carcinoma | | carcinoma of tongue | |
| RL95-2-Uterine | 0.3 | SCC-15-Squamous cell | 0.7 |
| carcinoma | | carcinoma of tongue | |
| HelaS3-Cervical | 2.3 | CAL 27-Squamous cell | 8.0 |
| adenocarcinoma | | carcinoma of tongue | |

TABLE CEF
Panel 4D

| | Rel. Exp. (%) | | Rel. Exp. (%) |
|---|---|---|---|
| | Ag3045, Run | | Ag3045, Run |
| Tissue Name | 162559632 | Tissue Name | 162559632 |
| Secondary Th1 act | 10.0 | HUVEC IL-1beta | 2.2 |
| Secondary Th2 act | 12.0 | HUVEC IFN gamma | 4.2 |
| Secondary Tr1 act | 21.9 | HUVEC TNF alpha + IFN | 3.2 |
| | | gamma | |
| Secondary Th1 rest | 3.3 | HUVEC TNF alpha + IL4 | 4.4 |
| Secondary Th2 rest | 5.6 | HUVEC IL-11 | 2.9 |
| Secondary Tr1 rest | 6.7 | Lung Microvascular EC | 6.9 |
| | | none | |
| Primary Th1 act | 13.1 | Lung Microvascular EC | 4.2 |
| | | TNF alpha + IL-1beta | |
| Primary Th2 act | 16.2 | Microvascular Dermal EC | 8.2 |
| | | none | |
| Primary Tr1 act | 17.0 | Microsvasular Dermal EC | 4.8 |
| | | TNF alpha + IL-1beta | |
| Primary Th1 rest | 47.0 | Bronchial epithelium | 1.1 |
| | | TNF alpha + IL1beta | |
| Primary Th2 rest | 21.9 | Small airway epithelium | 2.3 |
| | | none | |
| Primary Tr1 rest | 33.2 | Small airway epithelium | 34.2 |
| | | TNF alpha + IL-1beta | |
| CD45RA CD4 | 5.1 | Coronery artery SMC rest | 2.4 |
| lymphocyte act | | | |
| CD45RO CD4 | 11.6 | Coronery artery SMC | 2.1 |
| lymphocyte act | | TNF alpha + IL-1beta | |
| CD8 lymphocyte act | 8.6 | Astrocytes rest | 5.4 |
| Secondary CD8 | 8.1 | Astrocytes TNF alpha + | 3.6 |
| lymphocyte rest | | IL-1beta | |
| Secondary CD8 | 6.0 | KU-812 (Basophil) rest | 4.3 |
| lymphocyte act | | | |
| CD4 lymphocyte none | 2.3 | KU-812 (Basophil) | 14.1 |
| | | PMA/ionomycin | |
| 2ry Th1/Th2/Tr1_anti- | 12.0 | CCD1106 (Keratinocytes) | 6.0 |
| CD95 CH11 | | none | |
| LAK cells rest | 10.7 | CCD1106 (Keratinocytes) | 0.5 |
| | | TNF alpha + IL-1beta | |
| LAK cells IL-2 | 8.6 | Liver cirrhosis | 3.3 |
| LAK cells IL-2 + IL-12 | 8.5 | Lupus kidney | 1.7 |
| LAK cells IL-2 + IFN | 14.7 | NCI-H292 none | 100.0 |
| gamma | | | |
| LAK cells IL-2 + IL-18 | 18.0 | NCI-H292 IL-4 | 84.1 |
| LAK cells | 3.7 | NCI-H292 IL-9 | 96.6 |
| PMA/ionomycin | | | |
| NK Cells IL-2 rest | 6.8 | NCI-H292 IL-13 | 24.1 |
| Two Way MLR 3 day | 6.6 | NCI-H292 IFN gamma | 22.8 |
| Two Way MLR 5 day | 8.8 | HPAEC none | 4.9 |
| Two Way MLR 7 day | 5.7 | HPAEC TNF alpha + IL-1 | 3.9 |
| | | beta | |
| PBMC rest | 4.5 | Lung fibroblast none | 3.8 |
| PBMC PWM | 27.5 | Lung fibroblast TNF alpha + | 2.6 |
| | | IL-1beta | |
| PBMC PHA-L | 7.0 | Lung fibroblast IL-4 | 9.5 |
| Ramos (B cell) none | 6.6 | Lung fibroblast IL-9 | 5.5 |
| Ramos (B cell) | 34.4 | Lung fibroblast IL-13 | 6.0 |
| ionomycin | | | |
| B lymphocytes PWM | 29.1 | Lung fibroblast IFN | 5.2 |
| | | gamma | |
| B lymphocytes CD40L | 10.7 | Dermal fibroblast | 11.4 |
| and IL-4 | | CCD1070 rest | |
| EOL-1 dbcAMP | 6.3 | Dermal fibroblast | 32.5 |
| | | CCD1070 TNF alpha | |
| EOL-1 dbcAMP | 12.2 | Dermal fibroblast | 5.7 |
| PMA/ionomycin | | CCD1070 IL-1beta | |
| Dendritic cells none | 9.7 | Dermal fibroblast IFN | 3.6 |
| | | gamma | |
| Dendritic cells LPS | 6.1 | Dermal fibroblast IL-4 | 5.9 |
| Dendritic cells anti- | 11.7 | IBD Colitis 2 | 0.3 |
| CD40 | | | |
| Monocytes rest | 10.2 | IBD Crohn's | 0.4 |
| Monocytes LPS | 9.5 | Colon | 8.7 |
| Macrophages rest | 22.5 | Lung | 8.0 |
| Macrophages LPS | 2.4 | Thymus | 13.5 |
| HUVEC none | 5.7 | Kidney | 43.5 |
| HUVEC starved | 9.1 | | |

TABLE CEG
Panel CNS_1

| | Rel. Exp. (%) Ag3045, | | Rel. Exp. (%) Ag3045, |
|---|---|---|---|
| Tissue Name | Run171694539 | Tissue Name | Run 171694539 |
| BA4 Control | 0.9 | BA17 PSP | 0.0 |
| BA4 Control2 | 0.0 | BA17 PSP2 | 6.5 |
| BA4 | 9.3 | Sub Nigra Control | 2.6 |
| Alzheimer's2 | | | |
| BA4 Parkinson's | 55.1 | Sub Nigra Control2 | 0.0 |
| BA4 | 19.1 | Sub Nigra | 11.2 |
| Parkinson's2 | | Alzheimer's2 | |
| BA4 | 18.7 | Sub Nigra | 9.0 |
| Huntington's | | Parkinson's2 | |
| BA4 | 6.5 | Sub Nigra | 22.8 |
| Huntingson's2 | | Huntington's | |
| BA4 PSP | 0.0 | Sub Nigra | 21.9 |
| | | Huntington's2 | |
| BA4 PSP2 | 6.1 | Sub Nigra PSP2 | 2.3 |
| BA4 Depression | 15.1 | Sub Nigra | 0.0 |
| | | Depression | |
| BA4 | 6.6 | Sub Nigra | 3.0 |
| Depression2 | | Depression2 | |
| BA7 Control | 8.7 | Glob Palladus | 2.1 |
| | | Control | |
| BA7 Control2 | 0.0 | Glob Palladus | 0.0 |
| | | Control2 | |
| BA7 | 14.7 | Glob Palladus | 0.9 |
| Alzheimer's2 | | Alzheimer's | |
| BA7 Parkinson's | 26.8 | Glob Palladus | 2.8 |
| | | Alzheimer's2 | |
| BA7 | 16.3 | Glob Palladus | 61.6 |
| Parkinson's2 | | Parkinson's | |
| BA7 | 61.1 | Glob Palladus | 2.9 |
| Huntington's | | Parkinson's2 | |
| BA7 | 23.5 | Glob Palladus PSP | 0.0 |
| Huntington's2 | | | |
| BA7 PSP | 0.0 | Glob Palladus PSP2 | 0.8 |
| BA7 PSP2 | 11.6 | Glob Palladus | 0.0 |
| | | Depression | |
| BA7 Depression | 8.1 | Temp Pole Control | 0.0 |
| BA9 Control | 0.9 | Temp Pole Control2 | 1.3 |
| BA9 Control2 | 0.0 | Temp Pole | 0.0 |
| | | Alzheimer's | |
| BA9 Alzheimer's | 0.0 | Temp Pole | 5.6 |
| | | Alzheimer's2 | |
| BA9 | 20.6 | Temp Pole | 27.9 |
| Alzheimer's2 | | Parkinson's | |
| BA9 Parkinson's | 31.4 | Temp Pole | 2.0 |
| | | Parkinson's2 | |
| BA9 | 21.9 | Temp Pole | 29.1 |
| Parkinson's2 | | Huntington's | |
| BA9 | 23.0 | Temp Pole PSP | 0.0 |
| Huntington's | | | |
| BA9 | 15.2 | Temp Pole PSP2 | 0.9 |
| Huntington's2 | | | |
| BA9 PSP | 0.0 | Temp Pole | 5.5 |
| | | Depression2 | |
| BA9 PSP2 | 3.6 | Cing Gyr Control | 2.0 |
| BA9 Depression | 2.0 | Cing Gyr Control2 | 0.9 |
| BA9 | 2.9 | Cing Gyr | 0.0 |
| Depression2 | | Alzheimer's | |
| BA17 Control | 15.1 | Cing Gyr | 14.5 |
| | | Alzheimer's2 | |
| BA17 Control2 | 3.3 | Cing Gyr Parkinson's | 26.4 |
| BA17 | 15.5 | Cing Gyr | 12.0 |
| Alzheimer's2 | | Parkinson's2 | |
| BA17 | 100.0 | Cing Gyr | 46.0 |
| Parkinson's | | Huntington's | |
| BA17 | 54.0 | Cing Gyr | 19.8 |
| Parkinson's2 | | Huntington's2 | |
| BA17 | 32.3 | Cing Gyr PSP | 0.5 |
| Huntington's | | | |
| BA17 | 5.9 | Cing Gyr PSP2 | 1.9 |
| Huntington's2 | | | |
| BA17 | 4.0 | Cing Gyr Depression | 3.9 |
| Depression | | | |
| BA17 | 5.6 | Cing Gyr | 8.4 |
| Depression2 | | Depression2 | |

CNS_neurodegeneration_v1.0 Summary: Ag3045 The NOV90 gene is not differentially expressed in the postmortem brains of Alzheimer's diseased patients when compared to non-demented control. However, this panel does confirm the expression of this gene in the CNS of an independent sample of individuals. See panel 1 for a discussion of utility of this gene in the central nervous system.

Panel 1.3D Summary: Ag3045 Expression of the NOV90 gene shows a brain-preferential expression profile, and is expressed at moderate levels in all regions examined. Thus, this gene may be of utility as a small molecule target in neurologic disease.

In addition, significant expression is seen in a cluster of brain cancer cell lines. Thus, expression of this gene could be used to differentiate between this sample and other samples on this panel and as a marker to detect the presence of brain cancer. Furthermore, therapeutic modulation of the expression or function of this gene may be effective in the treatment of brain cancer.

Panel 2D Summary: Ag3045 The NOV90 gene is expressed at low levels in this panel. There is higher expression in gastric, breast, uterus and lung cancers then the normal samples from these organs. Expression of this gene could therefore be used as a diagnostic marker for gastric, lung, breast and uterine cancers. Furthermore, modulation of the gene product using small molecule inhibitors could be used for the treatment of these cancers.

Panel 3D Summary: A3045 The NOV90 gene is expressed at a low level in most of the cancer cell lines on this panel. Modulation of the gene product using small molecule inhibitors culd therefore be used for the treatment of cancer. Highest expression of this gene is seen in the cerebellum, confirming the results seen in Panel 1.3D.

Panel 4D Summary: Ag3045 The NOV90 gene, a serine/threonine-protein kinase homolog is expressed at moderate levels in pulmonary mucoepidermoid cells prepared under several conditions of cell activation: NCI-H292 none (CT=29.01), NCI-H292 IL-4 (CT=29.26), NCI-H292 IL-9 (CT=29.06), NCI-H292 IL-13 (CT=31.06), NCI-H292 IFN gamma (CT=31.14). Therefore, small molecule antagonists that block the function of the NOV90 gene product may be useful in several autoimmune and inflammatory diseases of the lung including, but not limited to, chronic obstructive pulmonary disease, asthma, and emphysema.

Panel CNS_{—}1 Summary: Ag3045 The expression in this panel confirms the presence of the NOV90 in the brain. Thus, this gene may be of utility as a small molecule target in neurologic disease.

NOV91

Expression of gene NOV91 was assessed using the primer-probe set Ag3018, described in Table CFA. Results of the RTQ-PCR runs are shown in Tables CFB, CFC and CFD.

TABLE CFA
Probe Name Ag3018

| | | | Start | |
|---|---|---|---|---|
| Primers | Sequences | Length | Position | SEQ ID NO: |
| Forward | 5′-ctgcagggttggagaaatg-3′ | 19 | 71 | 1354 |
| Probe | TET-5′-ccatcctgggcaaacccaaggat-3′-TAMRA | 23 | 107 | 1355 |
| Reverse | 5′-ctacacccatcatgttcacatg-3′ | 22 | 130 | 1356 |

TABLE CFB
CNS_neurodegeneration_v1.0

| | Rel. Exp. (%) Ag3018, | | Rel. Exp. (%) Ag3018, |
|---|---|---|---|
| Tissue Name | Run 209820676 | Tissue Name | Run 209820676 |
| AD 1 Hippo | 0.0 | Control (Path) 3 | 4.5 |
| | | Temporal Ctx | |
| AD 2 Hippo | 12.5 | Control (Path) 4 | 22.7 |
| | | Temporal Ctx | |
| AD 3 Hippo | 2.4 | AD 1 Occipital Ctx | 11.3 |
| AD 4 Hippo | 7.7 | AD 2 Occipital Ctx | 42.6 |
| | | (Missing) | |
| AD 5 hippo | 72.7 | AD 3 Occipital Ctx | 3.0 |
| AD 6 Hippo | 47.3 | AD 4 Occipital Ctx | 14.2 |
| Control 2 Hippo | 12.9 | AD 5 Occipital Ctx | 52.1 |
| Control 4 Hippo | 3.6 | AD 6 Occipital Ctx | 48.0 |
| Control (Path) 3 | 3.8 | Control 1 Occipital | 1.6 |
| Hippo | | Ctx | |
| AD 1 Temporal Ctx | 13.6 | Control 2 Occipital | 32.1 |
| | | Ctx | |
| AD 2 Temporal Ctx | 29.9 | Control 3 Occipital | 6.7 |
| | | Ctx | |
| AD 3 Temporal Ctx | 4.0 | Control 4 Occipital | 0.0 |
| | | Ctx | |
| AD 4 Temporal Ctx | 26.8 | Control (Path) 1 | 73.7 |
| | | Occipital Ctx | |
| AD 5 Inf Temporal | 100.0 | Control (Path) 2 | 12.6 |
| Ctx | | Occipital Ctx | |
| AD 5 SupTemporal | 54.7 | Control (Path) 3 | 1.7 |
| Ctx | | Occipital Ctx | |
| AD 6 Inf Temporal | 48.6 | Control (Path) 4 | 23.3 |
| Ctx | | Occipital Ctx | |
| AD 6 Sup Temporal | 49.0 | Control 1 Parietal | 6.5 |
| Ctx | | Ctx | |
| Control 1 Temporal | 1.7 | Control 2 Parietal | 57.8 |
| Ctx | | Ctx | |
| Control 2 Temporal | 9.9 | Control 3 Parietal | 7.3 |
| Ctx | | Ctx | |
| Control 3 Temporal | 14.4 | Control (Path) 1 | 57.8 |
| Ctx | | Parietal Ctx | |
| Control 4 Temporal | 2.3 | Control (Path) 2 | 21.2 |
| Ctx | | Parietal Ctx | |
| Control (Path) 1 | 30.4 | Control (Path) 3 | 3.6 |
| Temporal Ctx | | Parietal Ctx | |
| Control (Path) 2 | 36.9 | Control (Path) 4 | 17.6 |
| Temporal Ctx | | Parietal Ctx | |

TABLE CFC
Panel 1.3D

| | Rel. Exp. (%) Ag3018, | | Rel. Exp. (%) Ag3018, |
|---|---|---|---|
| Tissue Name | Run 167819112 | Tissue Name | Run 167819112 |
| Liver adenocarcinoma | 13.0 | Kidney (fetal) | 7.3 |
| Pancreas | 6.0 | Renal ca. 786-0 | 9.9 |
| Pancreatic ca. CAPAN 2 | 12.9 | Renal ca. A498 | 4.7 |
| Adrenal gland | 1.3 | Renal ca. RXF 393 | 21.2 |
| Thyroid | 1.3 | Renal ca. ACHN | 9.5 |
| Salivary gland | 0.0 | Renal ca. UO-31 | 4.1 |
| Pituitary gland | 7.9 | Renal ca. TK-10 | 12.7 |
| Brain (fetal) | 7.9 | Liver | 2.6 |
| Brain (whole) | 44.1 | Liver (fetal) | 2.9 |
| Brain (amygdala) | 3.2 | Liver ca. | 10.2 |
| | | (hepatoblast) HepG2 | |
| Brain (cerebellum) | 65.1 | Lung | 6.7 |
| Brain (hippocampus) | 9.8 | Lung (fetal) | 14.9 |
| Brain (substantia nigra) | 4.9 | Lung ca. (small cell) | 26.4 |
| | | LX-1 | |
| Brain (thalamus) | 8.8 | Lung ca. (small cell) | 4.6 |
| | | NCI-H69 | |
| Cerebral Cortex | 10.7 | Lung ca. (s.cell var.) | 75.3 |
| | | SHP-77 | |
| Spinal cord | 7.6 | Lung ca. (large | 2.9 |
| | | cell)NCI-H460 | |
| glio/astro U87-MG | 23.2 | Lung ca. (non-sm. | 55.1 |
| | | cell) A549 | |
| glio/astro U-118-MG | 37.9 | Lung ca. (non-s.cell) | 9.6 |
| | | NCI-H23 | |
| astrocytoma SW1783 | 35.1 | Lung ca. (non-s.cell) | 8.6 |
| | | HOP-62 | |
| neuro*; met SK-N-AS | 2.7 | Lung ca. (non-s.cl) | 11.0 |
| | | NCI-H522 | |
| astrocytoma SF-539 | 21.2 | Lung ca. (squam.) | 4.7 |
| | | SW 900 | |
| astrocytoma SNB-75 | 10.6 | Lung ca. (squam.) | 17.6 |
| | | NCI-H596 | |
| glioma SNB-19 | 24.8 | Mammary gland | 2.2 |
| glioma U251 | 28.5 | Breast ca.* (pl.ef) | 17.3 |
| | | MCF-7 | |
| glioma SF-295 | 34.6 | Breast ca.* (pl.ef) | 17.3 |
| | | MDA-MB-231 | |
| Heart (fetal) | 6.4 | Breast ca.* (pl.ef) | 6.5 |
| | | T47D | |
| Heart | 16.2 | Breast ca. BT-549 | 6.3 |
| Skeletal muscle (fetal) | 0.0 | Breast ca. MDA-N | 8.4 |
| Skeletal muscle | 11.7 | Ovary | 0.0 |
| Bone marrow | 9.2 | Ovarian ca. OVCAR-3 | 6.2 |
| Thymus | 27.7 | Ovarian ca. OVCAR-4 | 9.7 |
| Spleen | 5.6 | Ovarian ca. OVCAR-5 | 25.2 |
| Lymph node | 5.8 | Ovarian ca. OVCAR-8 | 20.3 |
| Colorectal | 15.0 | Ovarian ca. IGROV-1 | 9.2 |
| Stomach | 1.3 | Ovarian ca.* (ascites) | 26.6 |
| | | SK-OV-3 | |
| Small intestine | 1.7 | Uterus | 1.4 |
| Colon ca. SW480 | 14.7 | Placenta | 0.0 |
| Colon ca.* | 100.0 | Prostate | 4.2 |
| SW620(SW480 met) | | | |
| Colon ca. HT29 | 2.1 | Prostate ca.* (bone | 15.3 |
| | | met)PC-3 | |
| Colon ca. HCT-116 | 12.4 | Testis | 4.5 |
| Colon ca. CaCo-2 | 22.7 | Melanoma | 3.9 |
| | | Hs688(A).T | |
| Colon ca. | 7.0 | Melanoma* (met) | 9.8 |
| tissue(ODO3866) | | Hs688(B).T | |
| Colon ca. HCC-2998 | 35.6 | Melanoma UACC-62 | 4.1 |
| Gastric ca.* (liver met) | 19.6 | Melanoma M14 | 7.9 |
| NCI-N87 | | | |
| Bladder | 20.3 | Melanoma LOX | 11.0 |
| | | IMVI | |
| Trachea | 0.0 | Melanoma* (met) | 0.7 |
| | | SK-MEL-5 | |
| Kidney | 5.7 | Adipose | 0.0 |

TABLE CFD
Panel 4D

| | Rel. Exp. (%) | | Rel. Exp. (%) |
|---|---|---|---|
| | Ag3018, Run | | Ag3018, Run |
| Tissue Name | 164528110 | Tissue Name | 164528110 |
| Secondary Th1 act | 14.9 | HUVEC IL-1beta | 7.9 |
| Secondary Th2 act | 22.1 | HUVEC IFN gamma | 4.9 |
| Secondary Tr1 act | 28.9 | HUVEC TNF alpha + IFN | 4.5 |
| | | gamma | |
| Secondary Th1 rest | 4.1 | HUVEC TNF alpha + IL4 | 6.7 |
| Secondary Th2 rest | 5.9 | HUVEC IL-11 | 4.7 |
| Secondary Tr1 rest | 4.4 | Lung Microvascular EC | 10.9 |
| | | none | |
| Primary Th1 act | 19.5 | Lung Microvascular EC | 10.1 |
| | | TNF alpha + IL-1beta | |
| Primary Th2 act | 9.4 | Microvascular Dermal EC | 13.2 |
| | | none | |
| Primary Tr1 act | 13.6 | Microsvasular Dermal EC | 6.7 |
| | | TNF alpha + IL-1beta | |
| Primary Th1 rest | 45.7 | Bronchial epithelium | 6.2 |
| | | TNF alpha + IL1beta | |
| Primary Th2 rest | 18.6 | Small airway epithelium | 2.4 |
| | | none | |
| Primary Tr1 rest | 16.5 | Small airway epithelium | 17.6 |
| | | TNF alpha + IL-1beta | |
| CD45RA CD4 | 5.6 | Coronery artery SMC rest | 3.5 |
| lymphocyte act | | | |
| CD45RO CD4 | 17.1 | Coronery artery SMC | 1.0 |
| lymphocyte act | | TNF alpha + IL-1beta | |
| CD8 lymphocyte act | 16.5 | Astrocytes rest | 7.3 |
| Secondary CD8 | 9.6 | Astrocytes TNF alpha + | 3.1 |
| lymphocyte rest | | IL-1beta | |
| Secondary CD8 | 5.3 | KU-812 (Basophil) rest | 2.0 |
| lymphocyte act | | | |
| CD4 lymphocyte none | 4.6 | KU-812 (Basophil) | 6.7 |
| | | PMA/ionomycin | |
| 2ry Th1/Th2/Tr1_anti- | 12.0 | CCD1106 (Keratinocytes) | 4.1 |
| CD95 CH11 | | none | |
| LAK cells rest | 13.5 | CCD1106 (Keratinocytes) | 1.8 |
| | | TNF alpha + IL-1beta | |
| LAK cells IL-2 | 18.6 | Liver cirrhosis | 2.1 |
| LAK cells IL-2 + IL-12 | 14.6 | Lupus kidney | 1.7 |
| LAK cells IL-2 + IFN | 16.3 | NCI-H292 none | 13.5 |
| gamma | | | |
| LAK cells IL-2 + IL-18 | 33.9 | NCI-H292 IL-4 | 14.5 |
| LAK cells | 4.5 | NCI-H292 IL-9 | 23.3 |
| PMA/ionomycin | | | |
| NK Cells IL-2 rest | 17.3 | NCI-H292 IL-13 | 8.0 |
| Two Way MLR 3 day | 15.4 | NCI-H292 IFN gamma | 6.4 |
| Two Way MLR 5 day | 6.4 | HPAEC none | 4.6 |
| Two Way MLR 7 day | 6.3 | HPAEC TNF alpha + IL-1 | 6.4 |
| | | beta | |
| PBMC rest | 4.2 | Lung fibroblast none | 5.0 |
| PBMC PWM | 51.1 | Lung fibroblast TNF alpha + | 1.5 |
| | | IL-1beta | |
| PBMC PHA-L | 14.7 | Lung fibroblast IL-4 | 13.6 |
| Ramos (B cell) none | 24.3 | Lung fibroblast IL-9 | 7.2 |
| Ramos (B cell) | 100.0 | Lung fibroblast IL-13 | 13.1 |
| ionomycin | | | |
| B lymphocytes PWM | 34.4 | Lung fibroblast IFN | 6.8 |
| | | gamma | |
| B lymphocytes CD40L | 33.0 | Dermal fibroblast | 15.4 |
| and IL-4 | | CCD1070 rest | |
| EOL-1 dbcAMP | 13.3 | Dermal fibroblast | 44.8 |
| | | CCD1070 TNF alpha | |
| EOL-1 dbcAMP | 7.4 | Dermal fibroblast | 5.9 |
| PMA/ionomycin | | CCD1070 IL-1beta | |
| Dendritic cells none | 3.0 | Dermal fibroblast IFN | 3.6 |
| | | gamma | |
| Dendritic cells LPS | 8.8 | Dermal fibroblast IL-4 | 9.7 |
| Dendritic cells anti- | 6.0 | IBD Colitis 2 | 0.9 |
| CD40 | | | |
| Monocytes rest | 5.2 | IBD Crohn's | 0.0 |
| Monocytes LPS | 2.6 | Colon | 6.3 |
| Macrophages rest | 4.2 | Lung | 3.7 |
| Macrophages LPS | 1.3 | Thymus | 9.9 |
| HUVEC none | 5.3 | Kidney | 27.2 |
| HUVEC starved | 12.8 | | |

CNS_neurodegeneration_v1.0 Summary: Ag3018 This panel does not show differential expression of the NOV91 gene in Alzheimer's disease. However, this expression profile confirms the presence of this gene in the brain. Please see Panel 1.3D for discussion of utility of this gene in the central nervous system.

Panel 1.3D Summary: Ag3018 The NOV91 gene is expressed in the brain at low levels. D-dopachrome tautomerase has been implicated in the production of neuromelanin from the toxic quinone products of dopamine, and this pathway has been implicated in the death of dopaminergic neurons in Parkinson's disease. Thus, this gene may represent an excellent small molecule target for the treatment or prevention of Parkinson's disease.

In addition, significant expression is seen in a cluster of lung, brain, and colon cancer cell lines. Thus, expression of this gene could be used to differentiate between thes sample and other samples on this panel and as a marker to detect the presence of these cancers. Furthermore, therapeutic modulation of the expression or function of this gene may be effective in the treatment of lung, brain, and colon cancer.

References:

Matsunaga J, Sinha D, Solano F, Santis C, Wistow G, Hearing V. Macrophage migration inhibitory factor (MIF)—its role in catecholamine metabolism. Cell Mol Biol (Noisy-le-grand) November 1999;45(7):1035-40

Macrophage migration inhibitory factor (MIF) was originally identified several decades ago as a lymphokine-derived protein that inhibited monocyte migration. Recently, it has been reported that MIF has D-dopachrome tautomerase, phenylpyruvate tautomerase and thiol protein oxidoreductase activities, although the physiological significance of those activities is not yet clear. Here we show that MIF is able to catalyze the conversion of dopaminechrome and norepinephrinechrome, toxic quinone products of the neurotransmitters dopamine and norepinephrine, respectively, to indole derivatives that may serve as precursors to neuromelanin. Since MIF is highly expressed in human brain, these observations raise the possibility that MIF participates in a detoxification pathway for catecholamine products and could therefore have an important role for neural tissues. The potential role of MIF in the formation of neuromelanin from catecholamines is also an extremely interesting possibility.

Drukarch B, van Muiswinkel F L. Neuroprotection for Parkinson's disease: a new approach for a new millennium. Expert Opin Investig Drugs October 2001;10(10):1855-68

Parkinson's disease (PD) is the only neurogenerative disorder in which pharmacological intervention has resulted in a marked decrease in morbidity and a significant delay in mortality. However, the medium to long-term efficacy of this pharmacotherapy, mainly consisting of dopaminomimetics like L-dopa and dopamine receptor agonists, suffers greatly from the unrelenting progression of the disease process underlying PD, i.e., the degeneration of neuromelanin-containing, dopaminergic neurones in the substantia nigra. Efforts concentrated on understanding the mechanisms of dopaminergic cell death in Parkinson's disease have led to identification of a large variety of pathogenetic factors, including excessive release of oxygen free radicals during enzymatic dopamine breakdown, impairment of mitochondrial function, production of inflammatory mediators, loss of trophic support, and apoptosis. Therapeutic approaches aimed at correcting these abnormalities are currently being evaluated on their efficacy as neuroprotectants for PD. Here, we focus on the process of dopamine auto-oxidation, the chain of reactions leading to the formation of neuromelanin, as an often overlooked, yet obvious pathogenetic factor. In particular, we discuss the option of drug-mediated stimulation of endogenous mechanisms responsible for the detoxification of dopamine auto-oxidation products as a novel means of neuroprotection in Parkinson's disease.

Panel 4D Summary: Ag3018 The NOV91 gene, a D-dopachrome tautomerase homolog, is widely expressed in this panel, with highest expression in Ramos (B cells) activated by treatment with ionomycin (CT=31.28). Therefore, small molecule antagonists that block the function of the NOV91 gene product may be useful in several autoimmune and inflammatory diseases in which activated B cells can play major roles as sources of autoantibody-producing cells and also as powerful antigen-presenting cells, including, but not limited to, Crohn's disease, ulcerative colitis, multiple sclerosis, chronic obstructive pulmonary disease, asthma, emphysema, rheumatoid arthritis, lupus erythematosus, or psoriasis.

Panel 5 Islet Summary: Ag3018 Expression of this gene is low/undetectable (CTs>35) across all of the samples on this panel (data not shown).

NOV92

Expression of gene NOV92 was assessed using the primer-probe set Ag3048, described in Table CGA. Results of the RTQ-PCR runs are shown in Tables CGB, CGC, CGD and CGE.

TABLE CGA
Probe Name Ag3048

| | | | Start | |
|---|---|---|---|---|
| Primers | Sequences | Length | Position | SEQ ID NO: |
| Forward | 5′-cacaaccagctgacagacagt-3′ | 21 | 490 | 1357 |
| Probe | TET-5′-ccaccaccttcagcaagctgcatag-3′-TAMRA | 521 | 1358 | |
| Reverse | 5′-gggagagatccaggtattcaag-3′ | 22 | 547 | 1359 |

TABLE CGB
Panel 1.3D

| | Rel. Exp. (%) Ag3048, | | Rel. Exp. (%) Ag3048, |
|---|---|---|---|
| Tissue Name | Run 168017062 | Tissue Name | Run 168017062 |
| Liver adenocarcinoma | 2.0 | Kidney (fetal) | 0.6 |
| Pancreas | 0.0 | Renal ca. 786-0 | 3.8 |
| Pancreatic ca. CAPAN 2 | 0.3 | Renal ca. A498 | 17.7 |
| Adrenal gland | 0.0 | Renal ca. RXF 393 | 12.5 |
| Thyroid | 0.5 | Renal ca. ACHN | 0.1 |
| Salivary gland | 0.6 | Renal ca. UO-31 | 1.1 |
| Pituitary gland | 0.5 | Renal ca. TK-10 | 0.5 |
| Brain (fetal) | 0.9 | Liver | 0.0 |
| Brain (whole) | 2.4 | Liver (fetal) | 0.0 |
| Brain (amygdala) | 2.9 | Liver ca. | 0.2 |
| | | (hepatoblast) HepG2 | |
| Brain (cerebellum) | 7.0 | Lung | 1.4 |
| Brain (hippocampus) | 2.1 | Lung (fetal) | 7.1 |
| Brain (substantia nigra) | 4.5 | Lung ca. (small cell) | 0.2 |
| | | LX-1 | |
| Brain (thalamus) | 4.6 | Lung ca. (small cell) | 0.0 |
| | | NCI-H69 | |
| Cerebral Cortex | 1.0 | Lung ca. (s.cell var.) | 0.9 |
| | | SHP-77 | |
| Spinal cord | 7.6 | Lung ca. (large | 0.0 |
| | | cell)NCI-H460 | |
| glio/astro U87-MG | 2.8 | Lung ca. (non-sm. | 0.0 |
| | | cell) A549 | |
| glio/astro U-118-MG | 5.9 | Lung ca. (non-s.cell) | 0.4 |
| | | NCI-H23 | |
| astrocytoma SW1783 | 2.7 | Lung ca. (non-s.cell) | 41.2 |
| | | HOP-62 | |
| neuro*; met SK-N-AS | 1.1 | Lung ca. (non-s.cl) | 1.6 |
| | | NCI-H522 | |
| astrocytoma SF-539 | 100.0 | Lung ca. (squam.) | 0.0 |
| | | SW 900 | |
| astrocytoma SNB-75 | 41.2 | Lung ca. (squam.) | 0.0 |
| | | NCI-H596 | |
| glioma SNB-19 | 0.4 | Mammary gland | 15.9 |
| glioma U251 | 6.7 | Breast ca.* (pl.ef) | 3.5 |
| | | MCF-7 | |
| glioma SF-295 | 7.3 | Breast ca.* (pl.ef) | 2.0 |
| | | MDA-MB-231 | |
| Heart (fetal) | 5.1 | Breast ca.* (pl.ef) | 0.3 |
| | | T47D | |
| Heart | 1.3 | Breast ca. BT-549 | 0.0 |
| Skeletal muscle (fetal) | 4.7 | Breast ca. MDA-N | 0.3 |
| Skeletal muscle | 2.6 | Ovary | 3.8 |
| Bone marrow | 0.9 | Ovarian ca. OVCAR-3 | 0.2 |
| Thymus | 0.9 | Ovarian ca. OVCAR-4 | 0.2 |
| Spleen | 0.3 | Ovarian ca. OVCAR-5 | 4.3 |
| Lymph node | 0.0 | Ovarian ca. OVCAR-8 | 0.8 |
| Colorectal | 2.9 | Ovarian ca. IGROV-1 | 0.0 |
| Stomach | 0.8 | Ovarian ca.* (ascites) | 1.5 |
| | | SK-OV-3 | |
| Small intestine | 1.0 | Uterus | 0.6 |
| Colon ca. SW480 | 0.8 | Placenta | 0.9 |
| Colon ca.* | 1.9 | Prostate | 3.0 |
| SW620(SW480 met) | | | |
| Colon ca. HT29 | 0.3 | Prostate ca.* (bone | 1.7 |
| | | met)PC-3 | |
| Colon ca. HCT-116 | 0.0 | Testis | 3.9 |
| Colon ca. CaCo-2 | 0.2 | Melanoma | 32.8 |
| | | Hs688(A).T | |
| Colon ca. | 13.2 | Melanoma* (met) | 31.4 |
| tissue(ODO3866) | | Hs688(B).T | |
| Colon ca. HCC-2998 | 4.1 | Melanoma UACC-62 | 1.8 |
| Gastric ca.* (liver met) | 1.5 | Melanoma M14 | 0.0 |
| NCI-N87 | | | |
| Bladder | 3.0 | Melanoma LOX | 0.0 |
| | | IMVI | |
| Trachea | 3.7 | Melanoma* (met) | 0.0 |
| | | SK-MEL-5 | |
| Kidney | 0.0 | Adipose | 4.3 |

TABLE CGC
Panel 2D

| | Rel. Exp. (%) | | Rel. Exp. (%) |
|---|---|---|---|
| | Ag3048, | | Ag3048, |
| Tissue Name | Run 170858352 | Tissue Name | Run 170858352 |
| Normal Colon | 4.7 | Kidney Margin | 0.5 |
| | | 8120608 | |
| CC Well to Mod Diff | 12.0 | Kidney Cancer | 0.1 |
| (ODO3866) | | 8120613 | |
| CC Margin (ODO3866) | 1.5 | Kidney Margin | 0.4 |
| | | 8120614 | |
| CC Gr.2 rectosigmoid | 10.1 | Kidney Cancer | 13.1 |
| (ODO3868) | | 9010320 | |
| CC Margin (ODO3868) | 0.8 | Kidney Margin | 0.9 |
| | | 9010321 | |
| CC Mod Diff (ODO3920) | 8.4 | Normal Uterus | 0.5 |
| CC Margin (ODO3920) | 2.6 | Uterus Cancer 064011 | 1.0 |
| CC Gr.2 ascend colon | 13.6 | Normal Thyroid | 3.3 |
| (ODO3921) | | | |
| CC Margin (ODO3921) | 2.1 | Thyroid Cancer | 0.7 |
| | | 064010 | |
| CC from Partial | 3.1 | Thyroid Cancer | 4.5 |
| Hepatectomy (ODO4309) | | A302152 | |
| Mets | | | |
| Liver Margin (ODO4309) | 0.1 | Thyroid Margin | 0.8 |
| | | A302153 | |
| Colon mets to lung | 3.3 | Normal Breast | 20.0 |
| (OD04451-01) | | | |
| Lung Margin (OD04451- | 1.1 | Breast Cancer | 6.5 |
| 02) | | (OD04566) | |
| Normal Prostate 6546-1 | 4.1 | Breast Cancer | 10.4 |
| | | (OD04590-01) | |
| Prostate Cancer | 19.8 | Breast Cancer Mets | 11.4 |
| (OD04410) | | (OD04590-03) | |
| Prostate Margin | 4.5 | Breast Cancer | 1.0 |
| (OD04410) | | Metastasis | |
| | | (OD04655-05) | |
| Prostate Cancer | 9.7 | Breast Cancer 064006 | 23.2 |
| (OD04720-01) | | | |
| Prostate Margin | 7.5 | Breast Cancer 1024 | 25.7 |
| (OD04720-02) | | | |
| Normal Lung 061010 | 3.3 | Breast Cancer | 16.4 |
| | | 9100266 | |
| Lung Met to Muscle | 24.5 | Breast Margin | 24.0 |
| (ODO4286) | | 9100265 | |
| Muscle Margin | 1.7 | Breast Cancer | 32.3 |
| (ODO4286) | | A209073 | |
| Lung Malignant Cancer | 11.6 | Breast Margin | 33.2 |
| (OD03126) | | A2090734 | |
| Lung Margin (OD03126) | 3.1 | Normal Liver | 0.0 |
| Lung Cancer (OD04404) | 22.2 | Liver Cancer 064003 | 0.0 |
| Lung Margin (OD04404) | 7.2 | Liver Cancer 1025 | 0.0 |
| Lung Cancer (OD04565) | 36.1 | Liver Cancer 1026 | 4.2 |
| Lung Margin (OD04565) | 2.0 | Liver Cancer 6004-T | 0.0 |
| Lung Cancer (OD04237- | 12.6 | Liver Tissue 6004-N | 2.9 |
| 01) | | | |
| Lung Margin (OD04237- | 6.7 | Liver Cancer 6005-T | 2.2 |
| 02) | | | |
| Ocular Mel Met to Liver | 0.0 | Liver Tissue 6005-N | 0.0 |
| (ODO4310) | | | |
| Liver Margin (ODO4310) | 0.4 | Normal Bladder | 23.8 |
| Melanoma Mets to Lung | 0.0 | Bladder Cancer 1023 | 11.8 |
| (OD04321) | | | |
| Lung Margin (OD04321) | 1.7 | Bladder Cancer | 7.2 |
| | | A302173 | |
| Normal Kidney | 0.8 | Bladder Cancer | 12.7 |
| | | (OD04718-01) | |
| Kidney Ca, Nuclear grade | 0.3 | Bladder Normal | 4.9 |
| 2 (OD04338) | | Adjacent (OD04718- | |
| | | 03) | |
| Kidney Margin | 1.5 | Normal Ovary | 3.5 |
| (OD04338) | | | |
| Kidney Ca Nuclear grade | 0.2 | Ovarian Cancer | 100.0 |
| 1/2 (OD04339) | | 064008 | |
| Kidney Margin | 0.9 | Ovarian Cancer | 2.5 |
| (OD04339) | | (OD04768-07) | |
| Kidney Ca, Clear cell | 0.4 | Ovary Margin | 8.1 |
| type (OD04340) | | (OD04768-08) | |
| Kidney Margin | 1.0 | Normal Stomach | 1.5 |
| (OD04340) | | | |
| Kidney Ca, Nuclear grade | 10.3 | Gastric Cancer | 0.5 |
| 3 (OD04348) | | 9060358 | |
| Kidney Margin | 0.4 | Stomach Margin | 0.8 |
| (OD04348) | | 9060359 | |
| Kidney Cancer | 34.4 | Gastric Cancer | 7.5 |
| (OD04622-01) | | 9060395 | |
| Kidney Margin | 0.2 | Stomach Margin | 1.9 |
| (OD04622-03) | | 9060394 | |
| Kidney Cancer | 0.0 | Gastric Cancer | 27.0 |
| (OD04450-01) | | 9060397 | |
| Kidney Margin | 0.2 | Stomach Margin | 1.5 |
| (OD04450-03) | | 9060396 | |
| Kidney Cancer 8120607 | 9.0 | Gastric Cancer | 3.3 |
| | | 064005 | |

TABLE CGD
Panel 3D

| | Rel. Exp. (%) | | Rel. Exp. (%) |
|---|---|---|---|
| | Ag3048, Run | | Ag3048, Run |
| Tissue Name | 172133336 | Tissue Name | 172133336 |
| Daoy-Medulloblastoma | 6.5 | Ca Ski-Cervical epidermoid | 0.0 |
| | | carcinoma (metastasis) | |
| TE671-Medulloblastoma | 0.0 | ES-2-Ovarian clear cell | 0.0 |
| | | carcinoma | |
| D283 Med- | 0.0 | Ramos-Stimulated with | 0.0 |
| Medulloblastoma | | PMA/ionomycin 6 h | |
| PFSK-1-Primitive | 18.0 | Ramos-Stimulated with | 0.0 |
| Neuroectodermal | | PMA/ionomycin 14 h | |
| XF-498-CNS | 1.4 | MEG-01-Chronic | 2.6 |
| | | myelogenous leukemia | |
| | | (megokaryoblast) | |
| SNB-78-Glioma | 100.0 | Raji-Burkitt's lymphoma | 0.0 |
| SF-268-Glioblastoma | 0.0 | Daudi-Burkitt's lymphoma | 0.0 |
| T98G-Glioblastoma | 24.1 | U266-B-cell plasmacytoma | 0.0 |
| SK-N-SH- | 32.3 | CA46-Burkitt's lymphoma | 0.0 |
| Neuroblastoma | | | |
| (metastasis) | | | |
| SF-295-Glioblastoma | 2.0 | RL-non-Hodgkin's B-cell | 0.0 |
| | | lymphoma | |
| Cerebellum | 3.0 | JM1-pre-B-cell lymphoma | 0.0 |
| Cerebellum | 4.2 | Jurkat-T cell leukemia | 0.0 |
| NCI-H292- | 38.4 | TF-1-Erythroleukemia | 2.3 |
| Mucoepidermoid lung | | | |
| carcinoma | | | |
| DMS-114-Small cell | 1.7 | HUT 78-T-cell lymphoma | 0.0 |
| lung cancer | | | |
| DMS-79-Small cell lung | 3.0 | U937-Histiocytic lymphoma | 0.7 |
| cancer | | | |
| NCI-H146-Small cell | 3.3 | KU-812-Myelogenous | 0.0 |
| lung cancer | | leukemia | |
| NCI-H526-Small cell | 0.0 | 769-P-Clear cell renal | 0.0 |
| lung cancer | | carcinoma | |
| NCI-N417-Small cell | 0.0 | Caki-2-Clear cell renal | 2.5 |
| lung cancer | | carcinoma | |
| NCI-H82-Small cell | 0.0 | SW 839-Clear cell renal | 0.0 |
| lung cancer | | carcinoma | |
| NCI-H157-Squamous | 1.4 | G401-Wilms' tumor | 2.3 |
| cell lung cancer | | | |
| (metastasis) | | | |
| NCI-H1155-Large cell | 1.8 | Hs766T-Pancreatic | 1.5 |
| lung cancer | | carcinoma (LN metastasis) | |
| NCI-H1299-Large cell | 2.8 | CAPAN-1-Pancreatic | 0.0 |
| lung cancer | | adenocarcinoma (liver | |
| | | metastasis) | |
| NCI-H727-Lung | 0.0 | SU86.86-Pancreatic | 0.8 |
| carcinoid | | carcinoma (liver metastasis) | |
| NCI-UMC-11-Lung | 0.0 | BxPC-3-Pancreatic | 0.7 |
| carcinoid | | adenocarcinoma | |
| LX-1-Small cell lung | 0.0 | HPAC-Pancreatic | 0.0 |
| cancer | | adenocarcinoma | |
| Colo-205-Colon cancer | 7.3 | MIA PaCa-2-Pancreatic | 0.7 |
| | | carcinoma | |
| KM12-Colon cancer | 1.6 | CFPAC-1-Pancreatic ductal | 1.7 |
| | | adenocarcinoma | |
| KM20L2-Colon cancer | 0.0 | PANC-1-Pancreatic | 6.7 |
| | | epithelioid ductal carcinoma | |
| NCI-H716-Colon cancer | 0.0 | T24-Bladder carcinma | 6.1 |
| | | (transitional cell) | |
| SW-48-Colon | 0.6 | 5637-Bladder carcinoma | 2.1 |
| adenocarcinoma | | | |
| SW1116-Colon | 0.8 | HT-1197-Bladder carcinoma | 1.7 |
| adenocarcinoma | | | |
| LS 174T-Colon | 2.5 | UM-UC-3-Bladder carcinma | 0.7 |
| adenocarcinoma | | (transitional cell) | |
| SW-948-Colon | 0.0 | A204-Rhabdomyosarcoma | 5.4 |
| adenocarcinoma | | | |
| SW-480-Colon | 2.7 | HT-1080-Fibrosarcoma | 11.7 |
| adenocarcinoma | | | |
| NCI-SNU-5-Gastric | 1.6 | MG-63-Osteosarcoma | 0.0 |
| carcinoma | | | |
| KATO III-Gastric | 1.8 | SK-LMS-1-Leiomyosarcoma | 7.5 |
| carcinoma | | (vulva) | |
| NCI-SNU-16-Gastric | 1.4 | SJRH30-Rhabdomyosarcoma | 0.0 |
| carcinoma | | (met to bone marrow) | |
| NCI-SNU-1-Gastric | 0.0 | A431-Epidermoid carcinoma | 5.1 |
| carcinoma | | | |
| RF-1-Gastric | 0.0 | WM266-4-Melanoma | 16.6 |
| adenocarcinoma | | | |
| RF-48-Gastric | 0.9 | DU 145-Prostate carcinoma | 0.0 |
| adenocarcinoma | | (brain metastasis) | |
| MKN-45-Gastric | 5.6 | MDA-MB-468-Breast | 2.5 |
| carcinoma | | adenocarcinoma | |
| NCI-N87-Gastric | 2.3 | SCC-4-Squamous cell | 0.0 |
| carcinoma | | carcinoma of tongue | |
| OVCAR-5-Ovarian | 4.8 | SCC-9-Squamous cell | 0.0 |
| carcinoma | | carcinoma of tongue | |
| RL95-2-Uterine | 0.0 | SCC-15-Squamous cell | 0.6 |
| carcinoma | | carcinoma of tongue | |
| HelaS3-Cervical | 0.0 | CAL 27-Squamous cell | 0.5 |
| adenocarcinoma | | carcinoma of tongue | |

TABLE CGE
Panel 4D

| | Rel. Exp. (%) | | Rel. Exp. (%) |
|---|---|---|---|
| | Ag3048, Run | | Ag3048, Run |
| Tissue Name | 164315038 | Tissue Name | 164315038 |
| Secondary Th1 act | 0.4 | HUVEC IL-1beta | 0.0 |
| Secondary Th2 act | 0.0 | HUVEC IFN gamma | 1.9 |
| Secondary Tr1 act | 0.6 | HUVEC TNF alpha + IFN | 0.0 |
| | | gamma | |
| Secondary Th1 rest | 0.0 | HUVEC TNF alpha + IL4 | 1.2 |
| Secondary Th2 rest | 0.0 | HUVEC IL-11 | 0.3 |
| Secondary Tr1 rest | 0.0 | Lung Microvascular EC | 3.9 |
| | | none | |
| Primary Th1 act | 0.0 | Lung Microvascular EC | 1.2 |
| | | TNF alpha + IL-1beta | |
| Primary Th2 act | 0.0 | Microvascular Dermal EC | 0.6 |
| | | none | |
| Primary Tr1 act | 0.0 | Microsvasular Dermal EC | 1.4 |
| | | TNF alpha + IL-1beta | |
| Primary Th1 rest | 0.0 | Bronchial epithelium | 28.7 |
| | | TNF alpha + IL1beta | |
| Primary Th2 rest | 0.9 | Small airway epithelium | 39.2 |
| | | none | |
| Primary Tr1 rest | 0.6 | Small airway epithelium | 84.7 |
| | | TNF alpha + IL-1beta | |
| CD45RA CD4 | 8.6 | Coronery artery SMC rest | 17.9 |
| lymphocyte act | | | |
| CD45RO CD4 | 0.0 | Coronery artery SMC | 5.5 |
| lymphocyte act | | TNF alpha + IL-1beta | |
| CD8 lymphocyte act | 0.0 | Astrocytes rest | 6.1 |
| Secondary CD8 | 0.6 | Astrocytes TNF alpha + | 5.5 |
| lymphocyte rest | | IL-1beta | |
| Secondary CD8 | 0.6 | KU-812 (Basophil) rest | 0.7 |
| lymphocyte act | | | |
| CD4 lymphocyte none | 0.5 | KU-812 (Basophil) | 3.8 |
| | | PMA/ionomycin | |
| 2ry Th1/Th2/Tr1_anti- | 1.3 | CCD1106 (Keratinocytes) | 79.0 |
| CD95 CH11 | | none | |
| LAK cells rest | 0.0 | CCD1106 (Keratinocytes) | 97.3 |
| | | TNF alpha + IL-1beta | |
| LAK cells IL-2 | 0.0 | Liver cirrhosis | 4.3 |
| LAK cells IL-2 + IL-12 | 0.0 | Lupus kidney | 3.4 |
| LAK cells IL-2 + IFN | 0.0 | NCI-H292 none | 87.1 |
| gamma | | | |
| LAK cells IL-2 + IL-18 | 0.0 | NCI-H292 IL-4 | 77.4 |
| LAK cells | 0.0 | NCI-H292 IL-9 | 82.9 |
| PMA/ionomycin | | | |
| NK Cells IL-2 rest | 0.0 | NCI-H292 IL-13 | 54.0 |
| Two Way MLR 3 day | 0.0 | NCI-H292 IFN gamma | 49.3 |
| Two Way MLR 5 day | 0.0 | HPAEC none | 0.7 |
| Two Way MLR 7 day | 0.8 | HPAEC TNF alpha + IL-1 | 0.0 |
| | | beta | |
| PBMC rest | 0.8 | Lung fibroblast none | 35.1 |
| PBMC PWM | 0.0 | Lung fibroblast TNF alpha + | 7.0 |
| | | IL-1beta | |
| PBMC PHA-L | 2.2 | Lung fibroblast IL-4 | 46.0 |
| Ramos (B cell) none | 0.0 | Lung fibroblast IL-9 | 38.7 |
| Ramos (B cell) | 0.8 | Lung fibroblast IL-13 | 26.8 |
| ionomycin | | | |
| B lymphocytes PWM | 1.0 | Lung fibroblast IFN | 68.8 |
| | | gamma | |
| B lymphocytes CD40L | 0.0 | Dermal fibroblast | 100.0 |
| and IL-4 | | CCD1070 rest | |
| EOL-1 dbcAMP | 0.0 | Dermal fibroblast | 59.9 |
| | | CCD1070 TNF alpha | |
| EOL-1 dbcAMP | 0.0 | Dermal fibroblast | 39.8 |
| PMA/ionomycin | | CCD1070 IL-1beta | |
| Dendritic cells none | 1.8 | Dermal fibroblast IFN | 17.0 |
| | | gamma | |
| Dendritic cells LPS | 1.7 | Dermal fibroblast IL-4 | 30.6 |
| Dendritic cells anti- | 6.8 | IBD Colitis 2 | 0.0 |
| CD40 | | | |
| Monocytes rest | 1.2 | IBD Crohn's | 0.7 |
| Monocytes LPS | 0.0 | Colon | 2.0 |
| Macrophages rest | 1.9 | Lung | 36.1 |
| Macrophages LPS | 1.3 | Thymus | 0.6 |
| HUVEC none | 0.0 | Kidney | 1.7 |
| HUVEC starved | 2.9 | | |

Panel 1.3D Summary: Ag3048 The expression of the NOV92 gene appears to be highest in a sample derived from a brain cancer cell line (SF-539) (CT=29.4). In addition, there is substantial expression associated with samples derived from another brain cancer cell line, two melanoma cell lines and a lung cancer cell line. Thus, the expression of this gene could be used to distinguish SF-539 cells from other samples in the panel. Moreover, therapeutic modulation of this gene, through the use of small molecule drugs, protein therapeutics or antibodies might be of benefit in treatment of brain or lung cancer or melanoma.

This gene, a leucine-rich repeat homolog, is expressed at low levels in the CNS. The leucine-rich repeat region proteins have been implicated in axonal guidance. Therefore, this gene may be of therapeutic utility in the treatment of any CNS disorder involving neuronal loss, specifically by guiding/enhancing compensatory synaptogenesis and fiber outgrowth, including such clinical conditions as Alzheimer's, Parkinson's, or Huntington's diseases, stroke, head and spinal cord trauma, vascular dementia or spinocerebellar ataxia.

Panel 2D Summary: Ag3048 The expression of the NOV92 gene appears to be highest in a sample derived from an ovarian cancer (CT=29). In addition, there appears to be substantial expression associated with lung cancer, prostate cancer and colon cancer samples. Of note is the differential expression in the lung, colon and prostate cancer samples compared to their respective normal adjacent tissue. Thus, the expression of this gene could be used to distinguish this ovarian cancer sample from other samples in the panel. In addition, the expression of this gene could be used to distinguish colon, prostate or lung cancer samples from their normal adjacent tissue. Moreover, therapeutic modulation of this gene, through the use of small molecule drugs, protein therapeutics or antibodies might be beneficial in the treatment of ovarian, lung, prostate or colon cancer.

Panel 3D Summary: Ag3048 The expression of the NOV92 gene appears to be highest in a sample derived from a brain cancer cell line (SNB-78) (CT=30.2). In addition, there appears to be substantial expression associated with other brain cancer cell line samples and a lung cancer cell line sample. Thus, the expression of this gene could be used to distinguish SNB-78 cells from other samples in the panel. Moreover, therapeutic modulation of this gene, through the use of small molecule drugs, protein therapeutics or antibodies might be beneficial in the treatment of brain or lung cancer.

Panel 4D Summary: Ag3048 The NOV92 gene, a secreted leucine-rich repeat (LRR) protein, is expressed selectively at moderate levels (CT range 29-31) in several resting and cytokine-activated epithelial and connective tissue cells of lung and skin. Therefore, the NOV92 gene product may be useful as a therapeutic protein as well as a target for therapeutic antibodies and small molecules, each of which may prove beneficial in the reduction or elimination of the symptoms in patients with chronic obstructive pulmonary disease, asthma, emphysema, or psoriasis.

NOV93: IMP Dehydrogenase 1

Expression of gene NOV93 was assessed using the primer-probe sets Ag4520 and Ag2904, described in Tables CHA and CHB. Results of the RTQ-PCR runs are shown in Tables CHC, CHD, CHE, CHF, CHG, CHH and CHI.

TABLE CHA
Probe Name Ag4520

| | | | Start | |
|---|---|---|---|---|
| Primers | Sequences | Length | Position | SEQ ID NO: |
| Forward | 5′-ggtacccatgaggatgacaaat-3′ | 22 | 758 | 1360 |
| Probe | TET-5′-acctggacctgctcacccaggtag-3′-TAMRA | 24 | 783 | 1361 |
| Reverse | 5′-cgagtccaagcctatgacatt-3′ | 21 | 812 | 1362 |

TABLE CHB
Probe Name Ag2904

| | | | Start | |
|---|---|---|---|---|
| Primers | Sequences | Length | Position | SEQ ID NO: |
| Forward | 5′-atacttcaacgacggggataag-3′ | 22 | 1300 | 1363 |
| Probe | TET-5′-ctccatccaggacaaagggtccatt-3′-TAMRA | 25 | 1348 | 1364 |
| Reverse | 5′-aggtagggcacgaacttctg-3′ | 20 | 1373 | 1365 |

TABLE CHC
AI_comprehensive panel_v1.0

| | Rel. Exp. (%) | | Rel. Exp. (%) |
|---|---|---|---|
| | Ag4520, | | Ag4520, |
| Tissue Name | Run 219421380 | Tissue Name | Run 219421380 |
| 110967 COPD-F | 11.8 | 112427 Match Control | 63.7 |
| | | Psoriasis-F | |
| 110980 COPD-F | 12.1 | 1112418 Psoriasis-M | 12.5 |
| 110968 COPD-M | 11.0 | 112723 Match Control | 0.3 |
| | | Psoriasis-M | |
| 110977 COPD-M | 46.7 | 1112419 Psoriasis-M | 10.4 |
| 110989 Emphysema-F | 34.9 | 112424 Match Control | 11.6 |
| | | Psoriasis-M | |
| 1110992 Emphysema-F | 22.7 | 112420 Psoriasis-M | 52.1 |
| 110993 Emphysema-F | 21.9 | 112425 Match Control | 50.0 |
| | | Psoriasis-M | |
| 110994 Emphysema-F | 13.3 | 104689 (MF) OA | 30.8 |
| | | Bone-Backus | |
| 110995 Emphysema-F | 33.2 | 104690 (MF) Adj | 23.0 |
| | | “Normal” Bone-Backus | |
| 110996 Emphysema-F | 8.5 | 104691 (MF) OA | 22.4 |
| | | Synovium-Backus | |
| 110997 Asthma-M | 6.3 | 104692 (BA) OA | 3.4 |
| | | Cartilage-Backus | |
| 111001 Asthma-F | 32.5 | 104694 (BA) OA | 17.6 |
| | | Bone-Backus | |
| 111002 Asthma-F | 22.2 | 104695 (BA) Adj | 24.1 |
| | | “Normal” Bone-Backus | |
| 111003 Atopic | 18.9 | 104696 (BA) OA | 13.9 |
| Asthma-F | | Synovium-Backus | |
| 111004 Atopic | 20.9 | 104700 (SS) OA Bone- | 21.3 |
| Asthma-F | | Backus | |
| 111005 Atopic | 10.7 | 104701 (SS) Adj | 21.2 |
| Asthma-F | | “Normal” Bone-Backus | |
| 111006 Atopic | 3.6 | 104702 (SS) OA | 42.3 |
| Asthma-F | | Synovium-Backus | |
| 111417 Allergy-M | 13.1 | 117093 OA Cartilage | 27.9 |
| | | Rep7 | |
| 112347 Allergy-M | 2.6 | 112672 OA Bone5 | 39.0 |
| 112349 Normal Lung-F | 2.0 | 112673 OA Synovium5 | 14.6 |
| 112357 Normal Lung-F | 22.4 | 112674 OA Synovial | 16.6 |
| | | Fluid cells5 | |
| 112354 Normal Lung-M | 10.4 | 117100 OA Cartilage | 5.1 |
| | | Rep14 | |
| 112374 Crohns-F | 11.3 | 112756 OA Bone9 | 6.8 |
| 112389 Match | 18.6 | 112757 OA Synovium9 | 14.7 |
| Control Crohns-F | | | |
| 112375 Crohns-F | 8.5 | 112758 OA Synovial | 12.8 |
| | | Fluid Cells9 | |
| 112732 Match | 41.8 | 117125 RA Cartilage | 10.1 |
| Control Crohns-F | | Rep2 | |
| 112725 Crohns-M | 3.2 | 113492 Bone2 RA | 28.7 |
| 112387 Match | 13.5 | 113493 Synovium2 RA | 13.1 |
| Control Crohns-M | | | |
| 112378 Crohns-M | 1.6 | 113494 Syn Fluid Cells | 26.4 |
| | | RA | |
| 112390 Match | 49.7 | 113499 Cartilage4 RA | 29.3 |
| Control Crohns-M | | | |
| 112726 Crohns-M | 12.8 | 113500 Bone4 RA | 41.2 |
| 112731 Match | 16.7 | 113501 Synovium4 RA | 30.6 |
| Control Crohns-M | | | |
| 112380 Ulcer Col-F | 25.5 | 113502 Syn Fluid | 15.9 |
| | | Cells4 RA | |
| 112734 Match | 100.0 | 113495 Cartilage3 RA | 24.1 |
| Control Ulcer Col-F | | | |
| 112384 Ulcer Col-F | 48.6 | 113496 Bone3 RA | 31.9 |
| 112737 Match | 7.4 | 113497 Synovium3 RA | 13.7 |
| Control Ulcer Col-F | | | |
| 112386 Ulcer Col-F | 4.4 | 113498 Syn Fluid | 26.4 |
| | | Cells3 RA | |
| 112738 Match | 12.9 | 117106 Normal | 6.9 |
| Control Ulcer Col-F | | Cartilage Rep20 | |
| 112381 Ulcer Col-M | 2.0 | 113663 Bone3 Normal | 2.1 |
| 112735 Match | 12.0 | 113664 Synovium3 | 1.2 |
| Control Ulcer Col-M | | Normal | |
| 112382 Ulcer Col-M | 12.1 | 113665 Syn Fluid | 0.8 |
| | | Cells3 Normal | |
| 112394 Match | 4.5 | 117107 Normal | 13.5 |
| Control Ulcer Col-M | | Cartilage Rep22 | |
| 112383 Ulcer Col-M | 26.8 | 113667 Bone4 Normal | 18.3 |
| 112736 Match | 9.5 | 113668 Synovium4 | 17.6 |
| Control Ulcer Col-M | | Normal | |
| 112423 Psoriasis-F | 11.1 | 113669 Syn Fluid | 27.4 |
| | | Cells4 Normal | |

TABLE CHD
CNS_neurodegeneration_v1.0

| | Rel. Exp. (%) | Rel. Exp. (%) | | Rel. Exp. (%) | Rel. Exp. (%) |
|---|---|---|---|---|---|
| | Ag2904, Run | Ag4520, Run | Tissue | Ag2904, Run | Ag4520, Run |
| Tissue Name | 206485416 | 206954220 | Name | 206485416 | 206954220 |
| AD 1 Hippo | 29.3 | 31.9 | Control | 25.0 | 28.9 |
| | | | (Path) 3 | | |
| | | | Temporal | | |
| | | | Ctx | | |
| AD 2 Hippo | 40.6 | 34.2 | Control | 39.0 | 30.8 |
| | | | (Path) 4 | | |
| | | | Temporal | | |
| | | | Ctx | | |
| AD 3 Hippo | 34.2 | 26.1 | AD 1 | 27.4 | 23.7 |
| | | | Occipital | | |
| | | | Ctx | | |
| AD 4 Hippo | 29.5 | 19.1 | AD 2 | 0.0 | 0.0 |
| | | | Occipital | | |
| | | | Ctx | | |
| | | | (Missing) | | |
| AD 5 hippo | 60.7 | 58.2 | AD 3 | 33.0 | 24.8 |
| | | | Occipital | | |
| | | | Ctx | | |
| AD 6 Hippo | 80.7 | 92.7 | AD 4 | 24.5 | 26.2 |
| | | | Occipital | | |
| | | | Ctx | | |
| Control 2 | 25.0 | 30.8 | AD 5 | 41.5 | 30.1 |
| Hippo | | | Occipital | | |
| | | | Ctx | | |
| Control 4 | 53.2 | 40.9 | AD 6 | 30.6 | 52.1 |
| Hippo | | | Occipital | | |
| | | | Ctx | | |
| Control (Path) | 25.0 | 22.4 | Control 1 | 48.0 | 60.3 |
| 3 Hippo | | | Occipital | | |
| | | | Ctx | | |
| AD 1 Temporal | 52.1 | 81.2 | Control 2 | 33.4 | 41.5 |
| Ctx | | | Occipital | | |
| | | | Ctx | | |
| AD 2 Temporal | 24.3 | 21.5 | Control 3 | 25.3 | 23.3 |
| Ctx | | | Occipital | | |
| | | | Ctx | | |
| AD 3 Temporal | 32.3 | 26.1 | Control 4 | 30.8 | 25.3 |
| Ctx | | | Occipital | | |
| | | | Ctx | | |
| AD 4 Temporal | 40.6 | 49.7 | Control | 49.7 | 37.1 |
| Ctx | | | (Path) 1 | | |
| | | | Occipital | | |
| | | | Ctx | | |
| AD 5 Inf | 52.9 | 77.4 | Control | 15.5 | 12.3 |
| Temporal Ctx | | | (Path) 2 | | |
| | | | Occipital | | |
| | | | Ctx | | |
| AD 5 | 83.5 | 100.0 | Control | 39.2 | 45.1 |
| SupTemporal | | | (Path) 3 | | |
| Ctx | | | Occipital | | |
| | | | Ctx | | |
| AD 6 Inf | 77.4 | 85.3 | Control | 33.4 | 36.1 |
| Temporal Ctx | | | (Path) 4 | | |
| | | | Occipital | | |
| | | | Ctx | | |
| AD 6 Sup | 100.0 | 87.1 | Control 1 | 37.4 | 29.1 |
| Temporal Ctx | | | Parietal Ctx | | |
| Control 1 | 29.9 | 29.5 | Control 2 | 34.2 | 52.1 |
| Temporal Ctx | | | Parietal Ctx | | |
| Control 2 | 20.0 | 19.1 | Control 3 | 14.0 | 11.0 |
| Temporal Ctx | | | Parietal Ctx | | |
| Control 3 | 22.4 | 14.3 | Control | 43.2 | 30.1 |
| Temporal Ctx | | | (Path) 1 | | |
| | | | Parietal Ctx | | |
| Control 4 | 26.1 | 10.4 | Control | 32.8 | 33.4 |
| Temporal Ctx | | | (Path) 2 | | |
| | | | Parietal Ctx | | |
| Control (Path) | 46.3 | 25.7 | Control | 34.9 | 36.1 |
| 1 Temporal Ctx | | | (Path) 3 | | |
| | | | Parietal Ctx | | |
| Control (Path) | 34.2 | 34.2 | Control | 48.3 | 44.4 |
| 2 Temporal Ctx | | | (Path) 4 | | |
| | | | Parietal Ctx | | |

TABLE CHE
General_screening_panel_v1.4

| | Rel. Exp. (%) | Rel. Exp. (%) | | Rel. Exp. (%) | Rel. Exp. (%) |
|---|---|---|---|---|---|
| | Ag4520, Run | Ag4520, Run | | Ag4520, Run | Ag4520, Run |
| Tissue Name | 219274490 | 219288511 | Tissue Name | 219274490 | 219288511 |
| Adipose | 26.6 | 24.5 | Renal ca. TK-10 | 47.0 | 42.3 |
| Melanoma* | 14.1 | 12.9 | Bladder | 41.2 | 37.6 |
| Hs688(A).T | | | | | |
| Melanoma* | 13.6 | 13.3 | Gastric ca. (liver | 100.0 | 100.0 |
| Hs688(B).T | | | met.) NCI-N87 | | |
| Melanoma* | 8.5 | 11.2 | Gastric ca. | 14.4 | 13.5 |
| M14 | | | KATO III | | |
| Melanoma* | 17.2 | 18.6 | Colon ca. SW- | 5.0 | 5.3 |
| LOXIMVI | | | 948 | | |
| Melanoma* | 10.7 | 11.3 | Colon ca. SW480 | 18.6 | 20.9 |
| SK-MEL-5 | | | | | |
| Squamous cell | 4.9 | 6.6 | Colon ca.* | 9.0 | 9.0 |
| carcinoma | | | (SW480 met) | | |
| SCC-4 | | | SW620 | | |
| Testis Pool | 7.5 | 7.5 | Colon ca. HT29 | 10.1 | 10.0 |
| Prostate ca.* | 8.0 | 7.2 | Colon ca. HCT- | 16.7 | 14.6 |
| (bone met) | | | 116 | | |
| PC-3 | | | | | |
| Prostate Pool | 13.8 | 13.3 | Colon ca. CaCo-2 | 26.1 | 26.4 |
| Placenta | 18.3 | 19.5 | Colon cancer | 21.0 | 23.8 |
| | | | tissue | | |
| Uterus Pool | 9.0 | 9.0 | Colon ca. | 1.3 | 2.6 |
| | | | SW1116 | | |
| Ovarian ca. | 15.2 | 13.5 | Colon ca. Colo- | 3.5 | 3.7 |
| OVCAR-3 | | | 205 | | |
| Ovarian ca. | 28.9 | 28.3 | Colon ca. SW-48 | 4.3 | 4.5 |
| SK-OV-3 | | | | | |
| Ovarian ca. | 5.9 | 6.2 | Colon Pool | 31.9 | 30.1 |
| OVCAR-4 | | | | | |
| Ovarian ca. | 49.0 | 49.0 | Small Intestine | 29.7 | 32.5 |
| OVCAR-5 | | | Pool | | |
| Ovarian ca. | 16.3 | 15.0 | Stomach Pool | 15.6 | 15.2 |
| IGROV-1 | | | | | |
| Ovarian ca. | 6.9 | 6.0 | Bone Marrow | 13.4 | 13.9 |
| OVCAR-8 | | | Pool | | |
| Ovary | 11.3 | 10.0 | Fetal Heart | 20.3 | 21.8 |
| Breast ca. | 27.7 | 27.0 | Heart Pool | 11.3 | 15.4 |
| MCF-7 | | | | | |
| Breast ca. | 37.9 | 48.3 | Lymph Node | 26.8 | 30.1 |
| MDA-MB- | | | Pool | | |
| 231 | | | | | |
| Breast ca. BT | 52.9 | 53.2 | Fetal Skeletal | 7.5 | 10.7 |
| 549 | | | Muscle | | |
| Breast ca. | 69.7 | 74.7 | Skeletal Muscle | 20.9 | 24.3 |
| T47D | | | Pool | | |
| Breast ca. | 4.7 | 6.7 | Spleen Pool | 45.1 | 57.8 |
| MDA-N | | | | | |
| Breast Pool | 31.0 | 37.1 | Thymus Pool | 31.9 | 31.0 |
| Trachea | 31.0 | 28.5 | CNS cancer | 26.1 | 25.9 |
| | | | (glio/astro) U87- | | |
| | | | MG | | |
| Lung | 4.5 | 3.7 | CNS cancer | 21.3 | 24.0 |
| | | | (glio/astro) U- | | |
| | | | 118-MG | | |
| Fetal Lung | 68.3 | 100.0 | CNS cancer | 18.9 | 21.0 |
| | | | (neuro; met) SK- | | |
| | | | N-AS | | |
| Lung ca. NCI- | 0.5 | 0.6 | CNS cancer | 10.6 | 6.9 |
| N417 | | | (astro) SF-539 | | |
| Lung ca. LX-1 | 13.2 | 11.9 | CNS cancer | 48.0 | 47.0 |
| | | | (astro) SNB-75 | | |
| Lung ca. NCI- | 4.1 | 3.7 | CNS cancer | 14.7 | 13.4 |
| H146 | | | (glio) SNB-19 | | |
| Lung ca. | 6.3 | 6.3 | CNS cancer | 18.8 | 20.4 |
| SHP-77 | | | (glio) SF-295 | | |
| Lung ca. | 21.6 | 23.5 | Brain | 2.4 | 3.3 |
| A549 | | | (Amygdala) Pool | | |
| Lung ca. NCI- | 1.5 | 1.7 | Brain | 9.2 | 10.1 |
| H526 | | | (cerebellum) | | |
| Lung ca. NCI- | 29.7 | 29.5 | Brain (fetal) | 7.7 | 8.2 |
| H23 | | | | | |
| Lung ca. NCI- | 14.2 | 15.1 | Brain | 5.5 | 4.8 |
| H460 | | | (Hippocampus) | | |
| | | | Pool | | |
| Lung ca. | 17.4 | 3.6 | Cerebral Cortex | 5.4 | 3.7 |
| HOP-62 | | | Pool | | |
| Lung ca. NCI- | 20.3 | 25.5 | Brain (Substantia | 3.8 | 3.2 |
| H522 | | | nigra) Pool | | |
| Liver | 2.0 | 2.0 | Brain (Thalamus) | 4.4 | 4.7 |
| | | | Pool | | |
| Fetal Liver | 14.6 | 15.9 | Brain (whole) | 4.0 | 4.8 |
| Liver ca. | 16.2 | 18.8 | Spinal Cord Pool | 6.5 | 7.0 |
| HepG2 | | | | | |
| Kidney Pool | 36.3 | 44.4 | Adrenal Gland | 28.9 | 28.3 |
| Fetal Kidney | 31.9 | 29.1 | Pituitary gland | 3.3 | 2.5 |
| | | | Pool | | |
| Renal ca. 786-0 | 50.7 | 54.3 | Salivary Gland | 8.0 | 6.0 |
| Renal ca. | 28.9 | 27.4 | Thyroid (female) | 9.5 | 7.6 |
| A498 | | | | | |
| Renal ca. | 45.4 | 41.2 | Pancreatic ca. | 51.4 | 48.3 |
| ACHN | | | CAPAN2 | | |
| Renal ca. UO- | 31.9 | 31.6 | Pancreas Pool | 35.4 | 39.0 |
| 31 | | | | | |

TABLE CHF
Panel 1.3D

| | Rel. Exp. (%) Ag2904, | | Rel. Exp. (%) Ag2904, |
|---|---|---|---|
| Tissue Name | Run 162556421 | Tissue Name | Run 162556421 |
| Liver adenocarcinoma | 16.0 | Kidney (fetal) | 40.3 |
| Pancreas | 6.9 | Renal ca. 786-0 | 13.3 |
| Pancreatic ca. CAPAN 2 | 6.4 | Renal ca. A498 | 34.2 |
| Adrenal gland | 9.7 | Renal ca. RXF 393 | 16.3 |
| Thyroid | 8.1 | Renal ca. ACHN | 21.9 |
| Salivary gland | 7.4 | Renal ca. UO-31 | 6.2 |
| Pituitary gland | 2.1 | Renal ca. TK-10 | 9.5 |
| Brain (fetal) | 1.6 | Liver | 4.2 |
| Brain (whole) | 1.5 | Liver (fetal) | 11.3 |
| Brain (amygdala) | 6.6 | Liver ca. | 11.3 |
| | | (hepatoblast) HepG2 | |
| Brain (cerebellum) | 6.7 | Lung | 70.7 |
| Brain (hippocampus) | 7.6 | Lung (fetal) | 42.3 |
| Brain (substantia nigra) | 2.6 | Lung ca. (small cell) | 4.7 |
| | | LX-1 | |
| Brain (thalamus) | 5.0 | Lung ca. (small cell) | 1.6 |
| | | NCI-H69 | |
| Cerebral Cortex | 10.3 | Lung ca. (s.cell var.) | 2.3 |
| | | SHP-77 | |
| Spinal cord | 15.3 | Lung ca. (large | 6.9 |
| | | cell)NCI-H460 | |
| glio/astro U87-MG | 13.3 | Lung ca. (non-sm. | 3.5 |
| | | cell) A549 | |
| glio/astro U-118-MG | 3.4 | Lung ca. (non-s.cell) | 10.6 |
| | | NCI-H23 | |
| astrocytoma SW1783 | 6.1 | Lung ca. (non-s.cell) | 4.9 |
| | | HOP-62 | |
| neuro*; met SK-N-AS | 6.4 | Lung ca. (non-s.cl) | 6.9 |
| | | NCI-H522 | |
| astrocytoma SF-539 | 8.0 | Lung ca. (squam.) | 18.2 |
| | | SW 900 | |
| astrocytoma SNB-75 | 10.9 | Lung ca. (squam.) | 2.4 |
| | | NCI-H596 | |
| glioma SNB-19 | 7.3 | Mammary gland | 9.7 |
| glioma U251 | 3.4 | Breast ca.* (pl.ef) | 18.0 |
| | | MCF-7 | |
| glioma SF-295 | 8.1 | Breast ca.* (pl.ef) | 10.1 |
| | | MDA-MB-231 | |
| Heart (fetal) | 9.9 | Breast ca.* (pl.ef) | 4.1 |
| | | T47D | |
| Heart | 24.3 | Breast ca. BT-549 | 4.8 |
| Skeletal muscle (fetal) | 100.0 | Breast ca. MDA-N | 3.6 |
| Skeletal muscle | 9.8 | Ovary | 18.7 |
| Bone marrow | 35.4 | Ovarian ca. OVCAR-3 | 2.5 |
| Thymus | 80.7 | Ovarian ca. OVCAR-4 | 1.7 |
| Spleen | 50.0 | Ovarian ca. OVCAR-5 | 8.2 |
| Lymph node | 29.1 | Ovarian ca. OVCAR-8 | 3.5 |
| Colorectal | 42.3 | Ovarian ca. IGROV-1 | 4.9 |
| Stomach | 10.7 | Ovarian ca.* (ascites) | 5.2 |
| | | SK-OV-3 | |
| Small intestine | 25.3 | Uterus | 10.7 |
| Colon ca. SW480 | 2.4 | Placenta | 36.3 |
| Colon ca.* | 3.8 | Prostate | 10.5 |
| SW620(SW480 met) | | | |
| Colon ca. HT29 | 5.4 | Prostate ca.* (bone | 1.7 |
| | | met)PC-3 | |
| Colon ca. HCT-116 | 2.6 | Testis | 7.9 |
| Colon ca. CaCo-2 | 10.7 | Melanoma | 4.6 |
| | | Hs688(A).T | |
| Colon ca. | 21.6 | Melanoma* (met) | 6.2 |
| tissue(ODO3866) | | Hs688(B).T | |
| Colon ca. HCC-2998 | 10.4 | Melanoma UACC-62 | 0.9 |
| Gastric ca.* (liver met) | 27.2 | Melanoma M14 | 2.8 |
| NCI-N87 | | | |
| Bladder | 39.5 | Melanoma LOX | 2.9 |
| | | IMVI | |
| Trachea | 36.9 | Melanoma* (met) | 2.5 |
| | | SK-MEL-5 | |
| Kidney | 16.0 | Adipose | 27.4 |

TABLE CHG
Panel 2D

| | Rel. Exp. (%) | | Rel. Exp. (%) |
|---|---|---|---|
| | Ag2904, | | Ag2904, |
| Tissue Name | Run 162345750 | Tissue Name | Run 162345750 |
| Normal Colon | 58.6 | Kidney Margin | 5.3 |
| | | 8120608 | |
| CC Well to Mod Diff | 12.2 | Kidney Cancer | 7.9 |
| (ODO3866) | | 8120613 | |
| CC Margin (ODO3866) | 9.2 | Kidney Margin | 14.2 |
| | | 8120614 | |
| CC Gr.2 rectosigmoid | 50.0 | Kidney Cancer | 31.2 |
| (ODO3868) | | 9010320 | |
| CC Margin (ODO3868) | 5.6 | Kidney Margin | 27.2 |
| | | 9010321 | |
| CC Mod Diff (ODO3920) | 45.4 | Normal Uterus | 6.5 |
| CC Margin (ODO3920) | 27.2 | Uterus Cancer 064011 | 26.6 |
| CC Gr.2 ascend colon | 30.4 | Normal Thyroid | 10.7 |
| (ODO3921) | | | |
| CC Margin (ODO3921) | 9.9 | Thyroid Cancer | 14.2 |
| | | 064010 | |
| CC from Partial | 34.2 | Thyroid Cancer | 36.3 |
| Hepatectomy (ODO4309) | | A302152 | |
| Mets | | | |
| Liver Margin (ODO4309) | 19.1 | Thyroid Margin | 22.4 |
| | | A302153 | |
| Colon mets to lung | 52.1 | Normal Breast | 23.5 |
| (OD04451-01) | | | |
| Lung Margin (OD04451- | 43.2 | Breast Cancer | 6.8 |
| 02) | | (OD04566) | |
| Normal Prostate 6546-1 | 66.9 | Breast Cancer | 13.5 |
| | | (OD04590-01) | |
| Prostate Cancer | 22.4 | Breast Cancer Mets | 29.3 |
| (OD04410) | | (OD04590-03) | |
| Prostate Margin | 21.9 | Breast Cancer | 18.7 |
| (OD04410) | | Metastasis | |
| | | (OD04655-05) | |
| Prostate Cancer | 26.2 | Breast Cancer 064006 | 27.5 |
| (OD04720-01) | | | |
| Prostate Margin | 28.9 | Breast Cancer 1024 | 20.9 |
| (OD04720-02) | | | |
| Normal Lung 061010 | 100.0 | Breast Cancer | 12.2 |
| | | 9100266 | |
| Lung Met to Muscle | 27.2 | Breast Margin | 8.8 |
| (ODO4286) | | 9100265 | |
| Muscle Margin | 13.6 | Breast Cancer | 15.7 |
| (ODO4286) | | A209073 | |
| Lung Malignant Cancer | 32.5 | Breast Margin | 17.6 |
| (OD03126) | | A2090734 | |
| Lung Margin (OD03126) | 63.3 | Normal Liver | 13.4 |
| Lung Cancer (OD04404) | 28.3 | Liver Cancer 064003 | 13.9 |
| Lung Margin (OD04404) | 34.9 | Liver Cancer 1025 | 13.9 |
| Lung Cancer (OD04565) | 23.3 | Liver Cancer 1026 | 6.6 |
| Lung Margin (OD04565) | 49.0 | Liver Cancer 6004-T | 15.4 |
| Lung Cancer (OD04237- | 21.0 | Liver Tissue 6004-N | 9.6 |
| 01) | | | |
| Lung Margin (OD04237- | 55.9 | Liver Cancer 6005-T | 6.5 |
| 02) | | | |
| Ocular Mel Met to Liver | 3.6 | Liver Tissue 6005-N | 2.6 |
| (ODO4310) | | | |
| Liver Margin (ODO4310) | 14.9 | Normal Bladder | 46.7 |
| Melanoma Mets to Lung | 7.0 | Bladder Cancer 1023 | 10.4 |
| (OD04321) | | | |
| Lung Margin (OD04321) | 67.8 | Bladder Cancer | 16.0 |
| | | A302173 | |
| Normal Kidney | 47.0 | Bladder Cancer | 32.1 |
| | | (OD04718-01) | |
| Kidney Ca, Nuclear grade | 39.5 | Bladder Normal | 28.3 |
| 2 (OD04338) | | Adjacent (OD04718- | |
| | | 03) | |
| Kidney Margin | 45.4 | Normal Ovary | 2.4 |
| (OD04338) | | | |
| Kidney Ca Nuclear grade | 80.1 | Ovarian Cancer | 33.2 |
| 1/2 (OD04339) | | 064008 | |
| Kidney Margin | 28.1 | Ovarian Cancer | 80.7 |
| (OD04339) | | (OD04768-07) | |
| Kidney Ca, Clear cell | 67.8 | Ovary Margin | 11.5 |
| type (OD04340) | | (OD04768-08) | |
| Kidney Margin | 57.4 | Normal Stomach | 27.2 |
| (OD04340) | | | |
| Kidney Ca, Nuclear grade | 16.3 | Gastric Cancer | 6.9 |
| 3 (OD04348) | | 9060358 | |
| Kidney Margin | 60.7 | Stomach Margin | 10.3 |
| (OD04348) | | 9060359 | |
| Kidney Cancer | 27.5 | Gastric Cancer | 18.4 |
| (OD04622-01) | | 9060395 | |
| Kidney Margin | 5.6 | Stomach Margin | 22.8 |
| (OD04622-03) | | 9060394 | |
| Kidney Cancer | 28.5 | Gastric Cancer | 27.4 |
| (OD04450-01) | | 9060397 | |
| Kidney Margin | 34.4 | Stomach Margin | 9.2 |
| (OD04450-03) | | 9060396 | |
| Kidney Cancer 8120607 | 3.5 | Gastric Cancer | 77.9 |
| | | 064005 | |

TABLE CHH
Panel 4.1D

| | Rel. | Rel. | | Rel. | Rel. |
|---|---|---|---|---|---|
| | Exp. (%) | Exp. (%) | | Exp. (%) | Exp. (%) |
| | Ag4520, | Ag4520, | | Ag4520, | Ag4520, |
| | Run | Run | | Run | Run |
| Tissue Name | 198263642 | 219310605 | Tissue Name | 198263642 | 219310605 |
| Secondary Th1 act | 22.5 | 26.1 | HUVEC IL-1beta | 9.5 | 11.3 |
| Secondary Th2 act | 37.4 | 40.1 | HUVEC IFN | 17.0 | 15.6 |
| | | | gamma | | |
| Secondary Tr1 act | 22.8 | 22.7 | HUVEC TNF | 18.0 | 23.8 |
| | | | alpha + IFN | | |
| | | | gamma | | |
| Secondary Th1 rest | 7.8 | 6.3 | HUVEC TNF | 12.0 | 12.8 |
| | | | alpha + IL4 | | |
| Secondary Th2 rest | 8.7 | 11.7 | HUVEC IL-11 | 5.4 | 4.6 |
| Secondary Tr1 rest | 7.7 | 3.8 | Lung | 17.6 | 20.7 |
| | | | Microvascular EC | | |
| | | | none | | |
| Primary Th1 act | 35.8 | 22.4 | Lung | 19.9 | 23.5 |
| | | | Microvascular EC | | |
| | | | TNF alpha + IL- | | |
| | | | 1beta | | |
| Primary Th2 act | 36.3 | 39.0 | Microvascular | 8.1 | 8.7 |
| | | | Dermal EC none | | |
| Primary Tr1 act | 34.9 | 36.6 | Microsvasular | 9.1 | 11.1 |
| | | | Dermal EC | | |
| | | | TNF alpha + IL- | | |
| | | | 1beta | | |
| Primary Th1 rest | 3.8 | 2.6 | Bronchial | 5.3 | 5.0 |
| | | | epithelium | | |
| | | | TNF alpha + | | |
| | | | IL1beta | | |
| Primary Th2 rest | 3.4 | 3.1 | Small airway | 1.6 | 1.8 |
| | | | epithelium none | | |
| Primary Tr1 rest | 4.1 | 8.8 | Small airway | 10.6 | 9.7 |
| | | | epithelium | | |
| | | | TNF alpha + IL- | | |
| | | | 1beta | | |
| CD45RA CD4 | 15.1 | 14.0 | Coronery artery | 2.5 | 1.9 |
| lymphocyte act | | | SMC rest | | |
| CD45RO CD4 | 24.3 | 26.2 | Coronery artery | 3.7 | 3.4 |
| lymphocyte act | | | SMC TNF alpha + | | |
| | | | IL-1beta | | |
| CD8 lymphocyte | 13.4 | 16.3 | Astrocytes rest | 1.1 | 2.4 |
| act | | | | | |
| Secondary CD8 | 16.8 | 14.1 | Astrocytes | 7.1 | 5.8 |
| lymphocyte rest | | | TNF alpha + IL- | | |
| | | | 1beta | | |
| Secondary CD8 | 9.1 | 10.8 | KU-812 | 2.3 | 1.7 |
| lymphocyte act | | | (Basophil) rest | | |
| CD4 lymphocyte | 4.2 | 3.7 | KU-812 | 8.4 | 7.0 |
| none | | | (Basophil) | | |
| | | | PMA/ionomycin | | |
| 2ry | 14.7 | 13.1 | CCD1106 | 1.2 | 1.7 |
| Th1/Th2/Tr1_anti- | | | (Keratinocytes) | | |
| CD95 CH11 | | | none | | |
| LAK cells rest | 12.4 | 11.7 | CCD1106 | 6.6 | 3.6 |
| | | | (Keratinocytes) | | |
| | | | TNF alpha + IL- | | |
| | | | 1beta | | |
| LAK cells IL-2 | 14.3 | 15.5 | Liver cirrhosis | 2.8 | 3.6 |
| LAK cells IL-2 + IL- | 9.7 | 6.7 | NCI-H292 none | 2.6 | 2.9 |
| 12 | | | | | |
| LAK cells IL-2 + | 9.3 | 9.0 | NCI-H292 IL-4 | 2.6 | 3.6 |
| IFN gamma | | | | | |
| LAK cells IL-2 + | 12.8 | 11.7 | NCI-H292 IL-9 | 3.5 | 3.5 |
| IL-18 | | | | | |
| LAK cells | 25.0 | 32.1 | NCI-H292 IL-13 | 3.4 | 3.1 |
| PMA/ionomycin | | | | | |
| NK Cells IL-2 rest | 18.2 | 19.6 | NCI-H292 IFN | 3.6 | 4.2 |
| | | | gamma | | |
| Two Way MLR 3 | 20.7 | 22.7 | HPAEC none | 5.0 | 5.2 |
| day | | | | | |
| Two Way MLR 5 | 11.4 | 15.3 | HPAEC TNF | 19.9 | 25.0 |
| day | | | alpha + IL-1beta | | |
| Two Way MLR 7 | 13.2 | 14.6 | Lung fibroblast | 1.5 | 1.6 |
| day | | | none | | |
| PBMC rest | 5.0 | 4.7 | Lung fibroblast | 3.9 | 3.2 |
| | | | TNF alpha + IL- | | |
| | | | 1beta | | |
| PBMC PWM | 18.0 | 21.8 | Lung fibroblast | 1.2 | 1.4 |
| | | | IL-4 | | |
| PBMC PHA-L | 15.0 | 17.4 | Lung fibroblast | 1.1 | 2.4 |
| | | | IL-9 | | |
| Ramos (B cell) | 1.9 | 2.1 | Lung fibroblast | 1.5 | 1.8 |
| none | | | IL-13 | | |
| Ramos (B cell) | 3.0 | 3.0 | Lung fibroblast | 2.5 | 3.1 |
| ionomycin | | | IFN gamma | | |
| B lymphocytes | 10.4 | 12.7 | Dermal fibroblast | 3.4 | 1.8 |
| PWM | | | CCD1070 rest | | |
| B lymphocytes | 17.0 | 20.0 | Dermal fibroblast | 15.3 | 18.3 |
| CD40L and IL-4 | | | CCD1070 TNF | | |
| | | | alpha | | |
| EOL-1 dbcAMP | 9.8 | 17.0 | Dermal fibroblast | 4.6 | 4.6 |
| | | | CCD1070 IL- | | |
| | | | 1beta | | |
| EOL-1 dbcAMP | 37.4 | 32.3 | Dermal fibroblast | 3.3 | 2.9 |
| PMA/ionomycin | | | IFN gamma | | |
| Dendritic cells | 8.4 | 4.8 | Dermal fibroblast | 4.2 | 2.9 |
| none | | | IL-4 | | |
| Dendritic cells LPS | 20.0 | 25.2 | Dermal | 3.3 | 1.8 |
| | | | Fibroblasts rest | | |
| Dendritic cells anti- | 6.3 | 6.3 | Neutrophils | 51.8 | 56.6 |
| CD40 | | | TNFa + LPS | | |
| Monocytes rest | 10.8 | 9.8 | Neutrophils rest | 23.2 | 26.2 |
| Monocytes LPS | 100.0 | 100.0 | Colon | 1.7 | 1.6 |
| Macrophages rest | 7.6 | 6.8 | Lung | 4.0 | 1.8 |
| Macrophages LPS | 29.7 | 26.6 | Thymus | 7.9 | 8.7 |
| HUVEC none | 3.1 | 4.3 | Kidney | 6.3 | 3.6 |
| HUVEC starved | 10.4 | 8.0 | | | |

TABLE CHI
Panel 4D

| | Rel. Exp. (%) | | Rel. Exp. (%) |
|---|---|---|---|
| | Ag2904, Run | | Ag2904, Run |
| Tissue Name | 159078059 | Tissue Name | 159078059 |
| Secondary Th1 act | 52.5 | HUVEC IL-1beta | 14.8 |
| Secondary Th2 act | 65.5 | HUVEC IFN gamma | 32.5 |
| Secondary Tr1 act | 58.2 | HUVEC TNF alpha + IFN | 52.9 |
| | | gamma | |
| Secondary Th1 rest | 13.1 | HUVEC TNF alpha + IL4 | 28.9 |
| Secondary Th2 rest | 18.8 | HUVEC IL-11 | 8.4 |
| Secondary Tr1 rest | 16.6 | Lung Microvascular EC | 22.5 |
| | | none | |
| Primary Th1 act | 94.0 | Lung Microvascular EC | 39.2 |
| | | TNF alpha + IL-1beta | |
| Primary Th2 act | 66.4 | Microvascular Dermal EC | 20.2 |
| | | none | |
| Primary Tr1 act | 94.6 | Microsvasular Dermal EC | 30.4 |
| | | TNF alpha + IL-1beta | |
| Primary Th1 rest | 53.6 | Bronchial epithelium | 9.2 |
| | | TNF alpha + IL1beta | |
| Primary Th2 rest | 30.1 | Small airway epithelium | 6.1 |
| | | none | |
| Primary Tr1 rest | 18.8 | Small airway epithelium | 53.6 |
| | | TNF alpha + IL-1beta | |
| CD45RA CD4 | 24.7 | Coronery artery SMC rest | 16.7 |
| lymphocyte act | | | |
| CD45RO CD4 | 47.6 | Coronery artery SMC | 4.6 |
| lymphocyte act | | TNF alpha + IL-1beta | |
| CD8 lymphocyte act | 20.4 | Astrocytes rest | 4.8 |
| Secondary CD8 | 24.8 | Astrocytes TNF alpha + | 16.2 |
| lymphocyte rest | | IL-1beta | |
| Secondary CD8 | 27.5 | KU-812 (Basophil) rest | 6.6 |
| lymphocyte act | | | |
| CD4 lymphocyte none | 9.9 | KU-812 (Basophil) | 17.3 |
| | | PMA/ionomycin | |
| 2ry Th1/Th2/Tr1_anti- | 27.5 | CCD1106 (Keratinocytes) | 2.1 |
| CD95 CH11 | | none | |
| LAK cells rest | 28.9 | CCD1106 (Keratinocytes) | 4.2 |
| | | TNF alpha + IL-1beta | |
| LAK cells IL-2 | 33.2 | Liver cirrhosis | 5.6 |
| LAK cells IL-2 + IL-12 | 34.9 | Lupus kidney | 6.3 |
| LAK cells IL-2 + IFN | 47.3 | NCI-H292 none | 16.2 |
| gamma | | | |
| LAK cells IL-2 + IL-18 | 94.0 | NCI-H292 IL-4 | 13.4 |
| LAK cells | 48.3 | NCI-H292 IL-9 | 12.9 |
| PMA/ionomycin | | | |
| NK Cells IL-2 rest | 28.1 | NCI-H292 IL-13 | 6.2 |
| Two Way MLR 3 day | 53.2 | NCI-H292 IFN gamma | 13.6 |
| Two Way MLR 5 day | 20.9 | HPAEC none | 14.6 |
| Two Way MLR 7 day | 22.1 | HPAEC TNF alpha + IL-1 | 35.1 |
| | | beta | |
| PBMC rest | 8.4 | Lung fibroblast none | 3.6 |
| PBMC PWM | 100.0 | Lung fibroblast TNF alpha + | 6.4 |
| | | IL-1beta | |
| PBMC PHA-L | 34.9 | Lung fibroblast IL-4 | 5.8 |
| Ramos (B cell) none | 4.5 | Lung fibroblast IL-9 | 4.0 |
| Ramos (B cell) | 13.0 | Lung fibroblast IL-13 | 3.0 |
| ionomycin | | | |
| B lymphocytes PWM | 96.6 | Lung fibroblast IFN | 8.2 |
| | | gamma | |
| B lymphocytes CD40L | 84.7 | Dermal fibroblast | 9.5 |
| and IL-4 | | CCD1070 rest | |
| EOL-1 dbcAMP | 19.5 | Dermal fibroblast | 99.3 |
| | | CCD1070 TNF alpha | |
| EOL-1 dbcAMP | 74.7 | Dermal fibroblast | 15.2 |
| PMA/ionomycin | | CCD1070 IL-1beta | |
| Dendritic cells none | 13.4 | Dermal fibroblast IFN | 7.5 |
| | | gamma | |
| Dendritic cells LPS | 52.9 | Dermal fibroblast IL-4 | 6.7 |
| Dendritic cells anti- | 12.0 | IBD Colitis 2 | 1.3 |
| CD40 | | | |
| Monocytes rest | 22.2 | IBD Crohn's | 3.7 |
| Monocytes LPS | 67.8 | Colon | 15.7 |
| Macrophages rest | 18.9 | Lung | 11.3 |
| Macrophages LPS | 99.3 | Thymus | 28.3 |
| HUVEC none | 15.3 | Kidney | 31.9 |
| HUVEC starved | 34.2 | | |

AI_comprehensive panel_v1.0 Summary: Ag4520 The NOV93 gene is widely expressed among the samples on this panel, with highest expression in normal colon adjacent to diseased colon (CT=29), This widespread pattern of expression is consistent with expression in Panels 4D and 4.1D. Please see Panel 4.1D for discussion of utility of this gene in inflammation.

CNS_neurodegeneration_v1.0 Summary: Ag2904/Ag4520 The NOV93 gene, an IMP dehydrogenase homolog, shows a small but significant (p=0.02) upregulation in the postmortem Alzheimer's brain when compared to nondemented controls. IMP dehydrogenase is involved in purine metabolism, and has been implicated as a drug target for supressing the immune response, inflammation, and cerebral edema. The observed increase in the expression of this gene is in concordance with the evidence for the role of neuroinflammation in Alzheimer's disease. Therefore, the inhibition of this molecule may be of therapeutic benefit in Alzheimer's disease, head or spinal cord trauma, stroke, cerebral edema, or viral infections of the CNS.

References:

Hall I H, Wyrick S D. Cytoxicity of [(5,6-dichloro-9a-n-propyl-2,3,9,9a-tetrahydro-3-oxo-1H fluoren-7-yl)oxy]acetic acid, an agent known to reduce brain edema. Biomed Pharmacother 1996;50(1):19-23

A known agent, [(5,6-dichloro-9a-n-propyl-2,3,9,9a-tetrahydro-3-oxo-1H fluoren-7-yl)oxy]acetic acid, which blocks brain edema, was also shown to be a potent cytotoxic agent in leukemia cells. The major site of action of the agents appears to be in the de novo purine synthetic pathway in L1210 leukemic cells. Both PRPP amido transferase and IMP dehydrogenase activities were suppressed by the agent. The inhibition of both regulatory enzymes of the pathway along with the reduction of dihydrofolate reductase activity would account for the observed suppression of DNA and RNA syntheses and subsequent cancer cell death.

Senda M, Natsumeda Y. Tissue-differential expression of two distinct genes for human IMP dehydrogenase (E.C.1.1.1.205). Life Sci 1994;54(24):1917-26

Human IMP dehydrogenase (E.C. 1.1.1.205) is recently regarded as a potent targeting enzyme for immunosuppressive drugs. Tissue differential expressions of human type I and type II IMP dehydrogenase were investigated in sixteen human adult organs (heart, brain, placenta, lung, liver, skeletal muscle, kidney, pancreas, spleen, thymus, prostate, testis, ovary, small intestine, colon, peripheral blood leukocytes) and five human fetal organs (heart, brain, lung, liver, kidney) using Northern blot analysis. In all tissues examined in this study, the sizes of mRNAs of each isoform were identical, respectively. The 2.3 kb type II mRNA was shown predominantly, and the 3.5 kb type I mRNA level was lower than type II in most human tissues examined. In contrast, type I IMPDH gene expressed higher than type II in peripheral blood leukocytes, uniquely. We also demonstrated that both type I and type II IMPDH genes are widely distributed among various species by Southern blot analysis. Interestingly, type I IMPDH gene may have multiple gene families in primates. [dstone, Jan. 17, 2002]

General_screening_panel_v1.4 Summary: Ag4250 Two experiments with the same probe and primer set produce results that are in excellent agreement, with highest expression of the NOV93 gene in a gastric cancer cell line and fetal lung tissue. In addition, there appears to be substantial expression associated with breast cancer cell lines, lung cancer cell lines and renal cancer cell lines. Thus, the expression of this gene could be used to distinguish NCI-N87 and fetal lung tissue from the other samples in the panel. Moreover, therapeutic modulation of this gene, through the use of small molecule drugs, antibodies or protein therapeutics might be beneficial in the treatment of breast, lung or kidney cancer.

Among tissues with metabolic function, this gene has low-to-moderate levels of expression in adipose, liver, heart, skeletal muscle, adrenal, pituitary, thyroid and pancreas. Thus, this gene product may be a small molecule target for the treatment of metabolic and endocrine diseases, including obesity and Type 2 diabetes. This encodes a putative IMP dehydrogenase, which is involved in purine metabolism and has been implicated as a target for suppressing the immune response. Thus, this gene product may also be a treatment for Type 1 diabetes, in which insulin-secreting beta cells are destroyed by the autoimmune response against them. In addition, this gene appears to be differentially expressed in fetal (CT values=30) vs adult liver (CT value=33) and in fetal (CT values=27-28) vs. adult lung (CTs=33), and may be useful for the differentiation between the two sources of these tissues.

This molecule is also expressed at moderate to low levels in all CNS regions examined. Please see panel CNS_Neurodegeneration for a discussion of utility of this gene in the central nervous system.

Panel 1.3D Summary: Ag2904 Expression of the NOV93 gene is higher overall in normal tissues, with highest expression in fetal skeletal muscle. Furthermore, this gene is expressed at higher levels in fetal skeletal muscle (CT=29) when compared to expression in adult skeletal muscle (CT=32). Thus, expression of this gene could be used to differentiate between fetal skeletal muscle and other samples on this panel and between fetal and adult skeletal muscle.

Expression in the CNS is consistent with expression in previous panels. Please see CNS_neurodegeneration for discussion of utility of this gene in the central nervous system.

Among tissues with metabolic function, this gene is expressed at moderate to low levels in adipose, adrenal gland, pancreas, thyroid, and adult and fetal skeletal muscle, heart and liver. This widespread expression among these tissues suggests that this gene product may be useful for the diagnosis and/or treatment of metabolic disease, including obesity and diabetes.

Panel 2D Summary: Ag2904 The expression of the NOV93 gene appears to be highest in a sample derived from normal lung tissue. In addition, there appears to be substantial expression in most of the samples in the panel. Of note is the expression associated with normal lung tissue when compared to adjacent lung cancer tissue. Thus, the expression of this gene could be used to distinguish this sample of normal lung tissue from other samples in the panel. In addition, the expression of this gene could be used to distinguish normal lung tissue adjacent to cancer tissue. Moreover, therapeutic modulation of this gene, through the use of small molecule drugs, antibodies or protein therapeutics might be beneficial in the treatment of lung cancer.

Panel 4D/4.1D Summary: Ag2904/Ag4520 The NOV93 gene, a novel IMP dehydrogenase-like protein, is differentially expressed, as displayed in Panels 4.1D and 4D, in activated T cells, activated B cells, activated monocytes, activated macrophages, and activated dendritic cells. Small molecule antagonists of the previously characterized IMP dehydrogenase have been found to be useful in the treatment of several immunopathological states (See Allison and Eugui, 2001). Therefore, small molecule antagonists of the NOV93 gene product may reduce or eliminate the symptoms of autoimmune and inflammatory diseases, including Crohn's disease, ulcerative colitis, multiple sclerosis, chronic obstructive pulmonary disease, asthma, emphysema, rheumatoid arthritis, lupus erythematosus, or psoriasis.

References:

Allison A C, Eugui E M. Mycophenolate mofetil and its mechanisms of action. Immunopharmacology May 2000;47(2-3):85-118

Mycophenolate mofetil (MMF, CellCept(R)) is a prodrug of mycophenolic acid (MPA), an inhibitor of inosine monophosphate dehydrogenase (IMPDH). This is the rate-limiting enzyme in de novo synthesis of guanosine nucleotides. T- and B-lymphocytes are more dependent on this pathway than other cell types are. Moreover, MPA is a fivefold more potent inhibitor of the type II isoform of IMPDH, which is expressed in activated lymphocytes, than of the type I isoform of IMPDH, which is expressed in most cell types. MPA has therefore a more potent cytostatic effect on lymphocytes than on other cell types. This is the principal mechanism by which MPA exerts immunosuppressive effects. Three other mechanisms may also contribute to the efficacy of MPA in preventing allograft rejection and other applications. First, MPA can induce apoptosis of activated T-lymphocytes, which may eliminate clones of cells responding to antigenic stimulation. Second, by depleting guanosine nucleotides, MPA suppresses glycosylation and the expression of some adhesion molecules, thereby decreasing the recruitment of lymphocytes and monocytes into sites of inflammation and graft rejection. Third, by depleting guanosine nucleotides MPA also depletes tetrahydrobiopterin, a co-factor for the inducible form of nitric oxide synthase (iNOS). MPA therefore suppresses the production by iNOS of NO, and consequent tissue damage mediated by peroxynitrite. CellCept(R) suppresses T-lymphocytic responses to allogeneic cells and other antigens. The drug also suppresses primary, but not secondary, antibody responses. The efficacy of regimes including CellCept(R) in preventing allograft rejection, and in the treatment of rejection, is now firmly established. CellCept(R) is also efficacious in several experimental animal models of chronic rejection, and it is hoped that the drug will have the same effect in humans.

NOV94

Expression of gene NOV94 was assessed using the primer-probe set Ag2905, described in Table CIA. Results of the RTQ-PCR runs are shown in Tables CIB, CIC and CID.

TABLE CIA
Probe Name Ag2905

| | | | Start | |
|---|---|---|---|---|
| Primers | Sequences | Length | Position | SEQ ID NO: |
| Forward | 5′-gcagaataccacgatgacttct-3′ | 22 | 1468 | 1366 |
| Probe | TET-5′-agtcagcttacgtcgctgcctctgag-3′TAMRA | 26 | 1496 | 1367 |
| Reverse | 5′-gttcctggtgctgtaatgca-3′ | 20 | 1523 | 1368 |

TABLE CIB
Panel 1.3D

| | Rel. Exp. (%) Ag2905, | | Rel. Exp. (%) Ag2905, |
|---|---|---|---|
| Tissue Name | Run 161374149 | Tissue Name | Run 161374149 |
| Liver adenocarcinoma | 20.4 | Kidney (fetal) | 1.4 |
| Pancreas | 0.1 | Renal ca. 786-0 | 0.0 |
| Pancreatic ca. CAPAN 2 | 4.9 | Renal ca. A498 | 7.4 |
| Adrenal gland | 0.2 | Renal ca. RXF 393 | 9.0 |
| Thyroid | 0.9 | Renal ca. ACHN | 2.4 |
| Salivary gland | 0.6 | Renal ca. UO-31 | 5.5 |
| Pituitary gland | 0.4 | Renal ca. TK-10 | 13.7 |
| Brain (fetal) | 0.4 | Liver | 0.3 |
| Brain (whole) | 0.2 | Liver (fetal) | 0.8 |
| Brain (amygdala) | 1.2 | Liver ca. | 0.0 |
| | | (hepatoblast) HepG2 | |
| Brain (cerebellum) | 0.2 | Lung | 0.6 |
| Brain (hippocampus) | 1.0 | Lung (fetal) | 0.9 |
| Brain (substantia nigra) | 0.3 | Lung ca. (small cell) | 15.9 |
| | | LX-1 | |
| Brain (thalamus) | 1.0 | Lung ca. (small cell) | 1.2 |
| | | NCI-H69 | |
| Cerebral Cortex | 2.3 | Lung ca. (s.cell var.) | 27.7 |
| | | SHP-77 | |
| Spinal cord | 1.5 | Lung ca. (large | 2.0 |
| | | cell)NCI-H460 | |
| glio/astro U87-MG | 11.7 | Lung ca. (non-sm. | 8.0 |
| | | cell) A549 | |
| glio/astro U-118-MG | 0.0 | Lung ca. (non-s.cell) | 2.8 |
| | | NCI-H23 | |
| astrocytoma SW1783 | 0.2 | Lung ca. (non-s.cell) | 1.9 |
| | | HOP-62 | |
| neuro*; met SK-N-AS | 5.8 | Lung ca. (non-s.cl) | 5.4 |
| | | NCI-H522 | |
| astrocytoma SF-539 | 5.3 | Lung ca. (squam.) | 2.6 |
| | | SW 900 | |
| astrocytoma SNB-75 | 1.7 | Lung ca. (squam.) | 1.6 |
| | | NCI-H596 | |
| glioma SNB-19 | 0.1 | Mammary gland | 2.2 |
| glioma U251 | 0.0 | Breast ca.* (pl.ef) | 40.6 |
| | | MCF-7 | |
| glioma SF-295 | 0.3 | Breast ca.* (pl.ef) | 6.3 |
| | | MDA-MB-231 | |
| Heart (fetal) | 0.2 | Breast ca.* (pl.ef) | 0.0 |
| | | T47D | |
| Heart | 10.2 | Breast ca. BT-549 | 1.2 |
| Skeletal muscle (fetal) | 3.4 | Breast ca. MDA-N | 10.1 |
| Skeletal muscle | 0.7 | Ovary | 1.9 |
| Bone marrow | 1.0 | Ovarian ca. OVCAR-3 | 1.6 |
| Thymus | 20.2 | Ovarian ca. OVCAR-4 | 0.3 |
| Spleen | 0.8 | Ovarian ca. OVCAR-5 | 7.8 |
| Lymph node | 0.2 | Ovarian ca. OVCAR-8 | 10.2 |
| Colorectal | 1.0 | Ovarian ca. IGROV-1 | 1.5 |
| Stomach | 1.9 | Ovarian ca.* (ascites) | 9.4 |
| | | SK-OV-3 | |
| Small intestine | 2.3 | Uterus | 0.1 |
| Colon ca. SW480 | 6.0 | Placenta | 1.6 |
| Colon ca.* | 9.2 | Prostate | 3.5 |
| SW620(SW480 met) | | | |
| Colon ca. HT29 | 30.8 | Prostate ca.* (bone | 9.3 |
| | | met)PC-3 | |
| Colon ca. HCT-116 | 11.2 | Testis | 100.0 |
| Colon ca. CaCo-2 | 11.3 | Melanoma | 1.2 |
| | | Hs688(A).T | |
| Colon ca. | 2.4 | Melanoma* (met) | 2.0 |
| tissue(ODO3866) | | Hs688(B).T | |
| Colon ca. HCC-2998 | 7.2 | Melanoma UACC-62 | 1.5 |
| Gastric ca.* (liver met) | 40.6 | Melanoma M14 | 1.0 |
| NCI-N87 | | | |
| Bladder | 2.0 | Melanoma LOX | 0.6 |
| | | IMVI | |
| Trachea | 4.0 | Melanoma* (met) | 1.0 |
| | | SK-MEL-5 | |
| Kidney | 1.0 | Adipose | 0.4 |

TABLE CIC
Panel 2D

| | Rel. Exp. (%) | | Rel. Exp. (%) |
|---|---|---|---|
| | Ag2905, | | Ag2905, |
| Tissue Name | Run 161374481 | Tissue Name | Run 161374481 |
| Normal Colon | 15.3 | Kidney Margin | 1.1 |
| | | 8120608 | |
| CC Well to Mod Diff | 5.4 | Kidney Cancer | 0.5 |
| (ODO3866) | | 8120613 | |
| CC Margin (ODO3866) | 1.1 | Kidney Margin | 0.0 |
| | | 8120614 | |
| CC Gr.2 rectosigmoid | 3.1 | Kidney Cancer | 42.3 |
| (ODO3868) | | 9010320 | |
| CC Margin (ODO3868) | 1.1 | Kidney Margin | 2.0 |
| | | 9010321 | |
| CC Mod Diff (ODO3920) | 26.6 | Normal Uterus | 1.8 |
| CC Margin (ODO3920) | 3.6 | Uterus Cancer 064011 | 8.2 |
| CC Gr.2 ascend colon | 14.2 | Normal Thyroid | 9.2 |
| (ODO3921) | | | |
| CC Margin (ODO3921) | 6.2 | Thyroid Cancer | 16.0 |
| | | 064010 | |
| CC from Partial | 44.8 | Thyroid Cancer | 10.2 |
| Hepatectomy (ODO4309) | | A302152 | |
| Mets | | | |
| Liver Margin (ODO4309) | 8.4 | Thyroid Margin | 15.1 |
| | | A302153 | |
| Colon mets to lung | 11.0 | Normal Breast | 9.7 |
| (OD04451-01) | | | |
| Lung Margin (OD04451- | 0.9 | Breast Cancer | 1.2 |
| 02) | | (OD04566) | |
| Normal Prostate 6546-1 | 10.1 | Breast Cancer | 3.5 |
| | | (OD04590-01) | |
| Prostate Cancer | 52.9 | Breast Cancer Mets | 2.0 |
| (OD04410) | | (OD04590-03) | |
| Prostate Margin | 23.0 | Breast Cancer | 54.7 |
| (OD04410) | | Metastasis | |
| | | (OD04655-05) | |
| Prostate Cancer | 18.7 | Breast Cancer 064006 | 8.1 |
| (OD04720-01) | | | |
| Prostate Margin | 33.2 | Breast Cancer 1024 | 26.1 |
| (OD04720-02) | | | |
| Normal Lung 061010 | 12.9 | Breast Cancer | 30.6 |
| | | 9100266 | |
| Lung Met to Muscle | 29.3 | Breast Margin | 17.4 |
| (ODO4286) | | 9100265 | |
| Muscle Margin | 2.7 | Breast Cancer | 62.0 |
| (ODO4286) | | A209073 | |
| Lung Malignant Cancer | 3.4 | Breast Margin | 20.7 |
| (OD03126) | | A2090734 | |
| Lung Margin (OD03126) | 7.5 | Normal Liver | 11.7 |
| Lung Cancer (OD04404) | 35.6 | Liver Cancer 064003 | 6.7 |
| Lung Margin (OD04404) | 4.2 | Liver Cancer 1025 | 3.4 |
| Lung Cancer (OD04565) | 0.7 | Liver Cancer 1026 | 8.2 |
| Lung Margin (OD04565) | 2.2 | Liver Cancer 6004-T | 3.3 |
| Lung Cancer (OD04237- | 51.8 | Liver Tissue 6004-N | 15.3 |
| 01) | | | |
| Lung Margin (OD04237- | 0.4 | Liver Cancer 6005-T | 10.4 |
| 02) | | | |
| Ocular Mel Met to Liver | 41.8 | Liver Tissue 6005-N | 0.2 |
| (ODO4310) | | | |
| Liver Margin (ODO4310) | 4.4 | Normal Bladder | 16.7 |
| Melanoma Mets to Lung | 0.0 | Bladder Cancer 1023 | 6.3 |
| (OD04321) | | | |
| Lung Margin (OD04321) | 4.7 | Bladder Cancer | 40.6 |
| | | A302173 | |
| Normal Kidney | 7.1 | Bladder Cancer | 1.0 |
| | | (OD04718-01) | |
| Kidney Ca, Nuclear grade | 16.5 | Bladder Normal | 5.7 |
| 2 (OD04338) | | Adjacent (OD04718- | |
| | | 03) | |
| Kidney Margin | 7.4 | Normal Ovary | 1.3 |
| (OD04338) | | | |
| Kidney Ca Nuclear grade | 12.9 | Ovarian Cancer | 100.0 |
| 1/2 (OD04339) | | 064008 | |
| Kidney Margin | 4.6 | Ovarian Cancer | 18.3 |
| (OD04339) | | (OD04768-07) | |
| Kidney Ca, Clear cell | 5.8 | Ovary Margin | 0.7 |
| type (OD04340) | | (OD04768-08) | |
| Kidney Margin | 3.5 | Normal Stomach | 5.8 |
| (OD04340) | | | |
| Kidney Ca, Nuclear grade | 4.0 | Gastric Cancer | 0.9 |
| 3 (OD04348) | | 9060358 | |
| Kidney Margin | 4.4 | Stomach Margin | 4.8 |
| (OD04348) | | 9060359 | |
| Kidney Cancer | 0.7 | Gastric Cancer | 13.2 |
| (OD04622-01) | | 9060395 | |
| Kidney Margin | 1.3 | Stomach Margin | 8.3 |
| (OD04622-03) | | 9060394 | |
| Kidney Cancer | 11.9 | Gastric Cancer | 14.7 |
| (OD04450-01) | | 9060397 | |
| Kidney Margin | 8.8 | Stomach Margin | 2.1 |
| (OD04450-03) | | 9060396 | |
| Kidney Cancer 8120607 | 0.0 | Gastric Cancer | 17.1 |
| | | 064005 | |

TABLE CID
Panel 4D

| | Rel. Exp. (%) | | Rel. Exp. (%) |
|---|---|---|---|
| | Ag2905, Run | | Ag2905, Run |
| Tissue Name | 159772697 | Tissue Name | 159772697 |
| Secondary Th1 act | 12.8 | HUVEC IL-1beta | 8.2 |
| Secondary Th2 act | 6.5 | HUVEC IFN gamma | 9.1 |
| Secondary Tr1 act | 8.7 | HUVEC TNF alpha + IFN | 11.6 |
| | | gamma | |
| Secondary Th1 rest | 4.7 | HUVEC TNF alpha + IL4 | 12.2 |
| Secondary Th2 rest | 6.0 | HUVEC IL-11 | 9.0 |
| Secondary Tr1 rest | 6.7 | Lung Microvascular EC | 1.4 |
| | | none | |
| Primary Th1 act | 12.5 | Lung Microvascular EC | 3.1 |
| | | TNF alpha + IL-1beta | |
| Primary Th2 act | 10.6 | Microvascular Dermal EC | 24.3 |
| | | none | |
| Primary Tr1 act | 16.6 | Microvascular Dermal EC | 13.7 |
| | | TNF alpha + IL-1beta | |
| Primary Th1 rest | 24.5 | Bronchial epithelium | 1.0 |
| | | TNF alpha + IL1beta | |
| Primary Th2 rest | 10.2 | Small airway epithelium | 6.7 |
| | | none | |
| Primary Tr1 rest | 17.4 | Small airway epithelium | 29.1 |
| | | TNF alpha + IL-1beta | |
| CD45RA CD4 | 3.8 | Coronery artery SMC rest | 9.9 |
| lymphocyte act | | | |
| CD45RO CD4 | 8.9 | Coronery artery SMC | 6.0 |
| lymphocyte act | | TNF alpha + IL-1beta | |
| CD8 lymphocyte act | 5.1 | Astrocytes rest | 9.1 |
| Secondary CD8 | 8.2 | Astrocytes TNF alpha + | 5.4 |
| lymphocyte rest | | IL-1beta | |
| Secondary CD8 | 7.6 | KU-812 (Basophil) rest | 10.7 |
| lymphocyte act | | | |
| CD4 lymphocyte none | 1.0 | KU-812 (Basophil) | 21.6 |
| | | PMA/ionomycin | |
| 2ry Th1/Th2/Tr1_anti- | 8.9 | CCD1106 (Keratinocytes) | 30.4 |
| CD95 CH11 | | none | |
| LAK cells rest | 4.7 | CCD1106 (Keratinocytes) | 1.1 |
| | | TNF alpha + IL-1beta | |
| LAK cells IL-2 | 10.0 | Liver cirrhosis | 1.1 |
| LAK cells IL-2 + IL-12 | 11.2 | Lupus kidney | 1.0 |
| LAK cells IL-2 + IFN | 14.7 | NCI-H292 none | 35.4 |
| gamma | | | |
| LAK cells IL-2 + IL-18 | 11.7 | NCI-H292 IL-4 | 29.7 |
| LAK cells | 2.8 | NCI-H292 IL-9 | 49.0 |
| PMA/ionomycin | | | |
| NK Cells IL-2 rest | 5.0 | NCI-H292 IL-13 | 17.9 |
| Two Way MLR 3 day | 5.3 | NCI-H292 IFN gamma | 32.8 |
| Two Way MLR 5 day | 5.8 | HPAEC none | 4.8 |
| Two Way MLR 7 day | 3.4 | HPAEC TNF alpha + IL-1 | 5.0 |
| | | beta | |
| PBMC rest | 0.8 | Lung fibroblast none | 7.3 |
| PBMC PWM | 14.4 | Lung fibroblast TNF alpha + | 3.9 |
| | | IL-1beta | |
| PBMC PHA-L | 9.0 | Lung fibroblast IL-4 | 8.4 |
| Ramos (B cell) none | 17.4 | Lung fibroblast IL-9 | 10.9 |
| Ramos (B cell) | 100.0 | Lung fibroblast IL-13 | 8.8 |
| ionomycin | | | |
| B lymphocytes PWM | 31.2 | Lung fibroblast IFN | 17.1 |
| | | gamma | |
| B lymphocytes CD40L | 15.5 | Dermal fibroblast | 31.9 |
| and IL-4 | | CCD1070 rest | |
| EOL-1 dbcAMP | 0.0 | Dermal fibroblast | 39.2 |
| | | CCD1070 TNF alpha | |
| EOL-1 dbcAMP | 0.0 | Dermal fibroblast | 12.2 |
| PMA/ionomycin | | CCD1070 IL-1beta | |
| Dendritic cells none | 4.9 | Dermal fibroblast IFN | 6.4 |
| | | gamma | |
| Dendritic cells LPS | 3.0 | Dermal fibroblast IL-4 | 18.2 |
| Dendritic cells anti- | 4.9 | IBD Colitis 2 | 0.7 |
| CD40 | | | |
| Monocytes rest | 2.0 | IBD Crohn's | 0.0 |
| Monocytes LPS | 2.0 | Colon | 5.4 |
| Macrophages rest | 3.6 | Lung | 5.2 |
| Macrophages LPS | 1.8 | Thymus | 3.0 |
| HUVEC none | 19.2 | Kidney | 20.0 |
| HUVEC starved | 24.0 | | |

Panel 1.3D Summary: Ag2905 The expression of the NOV94 gene appears to be highest in a sample derived from normal testis tissue (CT=28.9). In addition, there is substantial expression associated with samples derived from colon cancer cell lines, lung cancer cell lines and breast cancer cell lines. Thus, the expression of this gene could be used to distinguish normal testis tissue from other samples in the panel. Moreover, therapeutic modulation of this gene, through the use of small molecule drugs, protein therapeutics or antibodies could be beneficial for the treatment of colon, lung or breast cancer.

In addition, this gene appears to be differentially expressed in fetal (CT value=37) vs adult heart (CT value=32), and may be useful for the differentiation between the two sources of heart tissue.

Panel 2D Summary: Ag2905 The expression of the NOV94 gene appears to be highest in a sample derived from an ovarian cancer (CT=30.5). In addition, there appears to be substantial expression associated with breast cancers, lung cancers, gastric cancers, prostate cancers and colon cancers. Thus, the expression of this gene could be used to distinguish this ovarian cancer sample from others in the panel. Moreover, therapeutic modulation of this gene, through the use of small molecule drugs, protein therapeutics or antibodies might be beneficial in the treatment of ovarian, breast, lung, gastric, prostate or colon cancer.

Panel 4D Summary: Ag2905 Low but significant expression of the NOV94 transcript is found predominantly in activated B cell lymphoma cell line (Ramos) and activated B cells (CTs=32-34). It is also found in HUVEC, keratinocytes, lung fibroblasts and the mucoepidermoid cell line H292. Therefore, targeting of this gene product with a small molecule drug therapeutic may modulate the functions of B cells and lead to the improvement of symptoms in autoimmune diseases such as lupus erythematosus, rheumatoid arthritis, hyperglobulinemia and other B cell disorders.

NOV95

Expression of gene NOV95 was assessed using the primer-probe set Ag3060, described in Table CJA. Results of the RTQ-PCR runs are shown in Tables CJB and CJC.

TABLE CJA
Probe Name Ag3060

| | | | Start | |
|---|---|---|---|---|
| Primers | Sequences | Length | Position | SEQ ID NO: |
| Forward | 5′-caaagattgcagcaatcgatag-3′ | 22 | 189 | 1369 |
| Probe | TET-5′-agtatacacgaggctttggccatcca-3′TAMRA | 26 | 219 | 1370 |
| Reverse | 5′-aggacagagctttcacaagtga-3′ | 22 | 245 | 1371 |

TABLE CJB
Panel 1.3D

| | Rel. Exp. (%) Ag3060, | | Rel. Exp. (%) Ag3060, |
|---|---|---|---|
| Tissue Name | Run 168016485 | Tissue Name | Run 168016485 |
| Liver adenocarcinoma | 0.2 | Kidney (fetal) | 0.3 |
| Pancreas | 0.2 | Renal ca. 786-0 | 0.1 |
| Pancreatic ca. CAPAN 2 | 0.0 | Renal ca. A498 | 0.1 |
| Adrenal gland | 0.1 | Renal ca. RXF 393 | 0.1 |
| Thyroid | 0.0 | Renal ca. ACHN | 0.1 |
| Salivary gland | 0.0 | Renal ca. UO-31 | 0.1 |
| Pituitary gland | 0.1 | Renal ca. TK-10 | 0.1 |
| Brain (fetal) | 0.1 | Liver | 0.0 |
| Brain (whole) | 0.1 | Liver (fetal) | 0.0 |
| Brain (amygdala) | 0.1 | Liver ca. | 0.1 |
| | | (hepatoblast) HepG2 | |
| Brain (cerebellum) | 0.1 | Lung | 0.0 |
| Brain (hippocampus) | 0.2 | Lung (fetal) | 0.1 |
| Brain (substantia nigra) | 0.1 | Lung ca. (small cell) | 0.1 |
| | | LX-1 | |
| Brain (thalamus) | 0.1 | Lung ca. (small cell) | 0.1 |
| | | NCI-H69 | |
| Cerebral Cortex | 0.0 | Lung ca. (s.cell var.) | 0.2 |
| | | SHP-77 | |
| Spinal cord | 0.1 | Lung ca. (large | 0.0 |
| | | cell)NCI-H460 | |
| glio/astro U87-MG | 0.0 | Lung ca. (non-sm. | 0.3 |
| | | cell) A549 | |
| glio/astro U-118-MG | 0.0 | Lung ca. (non-s.cell) | 0.1 |
| | | NCI-H23 | |
| astrocytoma SW1783 | 0.2 | Lung ca. (non-s.cell) | 0.1 |
| | | HOP-62 | |
| neuro*; met SK-N-AS | 0.1 | Lung ca. (non-s.cl) | 0.2 |
| | | NCI-H522 | |
| astrocytoma SF-539 | 0.1 | Lung ca. (squam.) | 0.1 |
| | | SW 900 | |
| astrocytoma SNB-75 | 0.2 | Lung ca. (squam.) | 0.1 |
| | | NCI-H596 | |
| glioma SNB-19 | 0.1 | Mammary gland | 0.1 |
| glioma U251 | 0.3 | Breast ca.* (pl.ef) | 0.1 |
| | | MCF-7 | |
| glioma SF-295 | 0.2 | Breast ca.* (pl.ef) | 0.1 |
| | | MDA-MB-231 | |
| Heart (fetal) | 3.3 | Breast ca.* (pl.ef) | 100.0 |
| | | T47D | |
| Heart | 0.0 | Breast ca. BT-549 | 0.0 |
| Skeletal muscle (fetal) | 0.0 | Breast ca. MDA-N | 0.0 |
| Skeletal muscle | 0.0 | Ovary | 0.0 |
| Bone marrow | 0.0 | Ovarian ca. OVCAR-3 | 0.2 |
| Thymus | 0.1 | Ovarian ca. OVCAR-4 | 0.2 |
| Spleen | 0.1 | Ovarian ca. OVCAR-5 | 0.6 |
| Lymph node | 0.1 | Ovarian ca. OVCAR-8 | 0.1 |
| Colorectal | 0.0 | Ovarian ca. IGROV-1 | 0.0 |
| Stomach | 0.0 | Ovarian ca.* (ascites) | 0.2 |
| | | SK-OV-3 | |
| Small intestine | 0.0 | Uterus | 0.1 |
| Colon ca. SW480 | 0.1 | Placenta | 0.0 |
| Colon ca.* | 0.2 | Prostate | 0.0 |
| SW620(SW480 met) | | | |
| Colon ca. HT29 | 0.1 | Prostate ca.* (bone | 0.1 |
| | | met)PC-3 | |
| Colon ca. HCT-116 | 0.1 | Testis | 3.1 |
| Colon ca. CaCo-2 | 0.1 | Melanoma | 0.0 |
| | | Hs688(A).T | |
| Colan ca. | 0.0 | Melanoma* (met) | 0.1 |
| tissue(ODO3866) | | Hs688(B).T | |
| Colon ca. HCC-2998 | 0.2 | Melanoma UACC-62 | 0.0 |
| Gastric ca.* (liver met) | 0.1 | Melanoma M14 | 0.1 |
| NCI-N87 | | | |
| Bladder | 0.1 | Melanoma LOX | 0.0 |
| | | IMVI | |
| Trachea | 0.1 | Melanoma* (met) | 0.0 |
| | | SK-MEL-5 | |
| Kidney | 0.1 | Adipose | 0.1 |

TABLE CJC
Panel 4D

| | Rel. Exp. (%) | | Rel. Exp. (%) |
|---|---|---|---|
| | Ag3060, Run | | Ag3060, Run |
| Tissue Name | 164317425 | Tissue Name | 164317425 |
| Secondary Th1 act | 22.7 | HUVEC IL-1beta | 7.2 |
| Secondary Th2 act | 25.7 | HUVEC IFN gamma | 15.1 |
| Secondary Tr1 act | 40.3 | HUVEC TNF alpha + IFN | 12.3 |
| | | gamma | |
| Secondary Th1 rest | 6.1 | HUVEC TNF alpha + IL4 | 10.4 |
| Secondary Th2 rest | 9.3 | HUVEC IL-11 | 4.8 |
| Secondary Tr1 rest | 11.7 | Lung Microvascular EC | 6.0 |
| | | none | |
| Primary Th1 act | 29.5 | Lung Microvascular EC | 9.7 |
| | | TNF alpha + IL-1beta | |
| Primary Th2 act | 24.1 | Microvascular Dermal EC | 11.8 |
| | | none | |
| Primary Tr1 act | 33.4 | Microsvasular Dermal EC | 9.6 |
| | | TNF alpha + IL-1beta | |
| Primary Th1 rest | 52.9 | Bronchial epithelium | 20.0 |
| | | TNF alpha + IL1beta | |
| Primary Th2 rest | 26.2 | Small airway epithelium | 5.0 |
| | | none | |
| Primary Tr1 rest | 17.4 | Small airway epithelium | 38.4 |
| | | TNF alpha + IL-1beta | |
| CD45RA CD4 | 13.8 | Coronery artery SMC rest | 12.1 |
| lymphocyte act | | | |
| CD45RO CD4 | 23.0 | Coronery artery SMC | 8.7 |
| lymphocyte act | | TNF alpha + IL-1beta | |
| CD8 lymphocyte act | 26.4 | Astrocytes rest | 9.0 |
| Secondary CD8 | 25.9 | Astrocytes TNF alpha + | 5.6 |
| lymphocyte rest | | IL-1beta | |
| Secondary CD8 | 15.1 | KU-812 (Basophil) rest | 22.7 |
| lymphocyte act | | | |
| CD4 lymphocyte none | 7.1 | KU-812 (Basophil) | 67.8 |
| | | PMA/ionomycin | |
| 2ry Th1/Th2/Tr1_anti- | 13.4 | CCD1106 (Keratinocytes) | 10.3 |
| CD95 CH11 | | none | |
| LAK cells rest | 15.0 | CCD1106 (Keratinocytes) | 5.8 |
| | | TNF alpha + IL-1beta | |
| LAK cells IL-2 | 18.7 | Liver cirrhosis | 2.6 |
| LAK cells IL-2 + IL-12 | 14.5 | Lupus kidney | 1.0 |
| LAK cells IL-2 + IFN | 24.1 | NCI-H292 none | 22.1 |
| gamma | | | |
| LAK cells IL-2 + IL-18 | 19.5 | NCI-H292 IL-4 | 21.8 |
| LAK cells | 11.7 | NCI-H292 IL-9 | 28.7 |
| PMA/ionomycin | | | |
| NK Cells IL-2 rest | 14.2 | NCI-H292 IL-13 | 15.5 |
| Two Way MLR 3 day | 14.0 | NCI-H292 IFN gamma | 27.4 |
| Two Way MLR 5 day | 14.5 | HPAEC none | 7.1 |
| Two Way MLR 7 day | 13.7 | HPAEC TNF alpha + IL-1 | 11.5 |
| | | beta | |
| PBMC rest | 4.2 | Lung fibroblast none | 8.9 |
| PBMC PWM | 38.2 | Lung fibroblast TNF alpha + | 10.8 |
| | | IL-1beta | |
| PBMC PHA-L | 21.3 | Lung fibroblast IL-4 | 14.6 |
| Ramos (B cell) none | 14.4 | Lung fibroblast IL-9 | 13.9 |
| Ramos (B cell) | 67.8 | Lung fibroblast IL-13 | 11.0 |
| ionomycin | | | |
| B lymphocytes PWM | 100.0 | Lung fibroblast IFN | 20.9 |
| | | gamma | |
| B lymphocytes CD40L | 27.2 | Dermal fibroblast | 16.3 |
| and IL-4 | | CCD1070 rest | |
| EOL-1 dbcAMP | 13.4 | Dermal fibroblast | 32.3 |
| | | CCD1070 TNF alpha | |
| EOL-1 dbcAMP | 14.8 | Dermal fibroblast | 7.3 |
| PMA/ionomycin | | CCD1070 IL-1beta | |
| Dendritic cells none | 11.4 | Dermal fibroblast IFN | 10.2 |
| | | gamma | |
| Dendritic cells LPS | 15.9 | Dermal fibroblast IL-4 | 18.2 |
| Dendritic cells anti- | 10.8 | IBD Colitis 2 | 0.5 |
| CD40 | | | |
| Monocytes rest | 10.6 | IBD Crohn's | 1.2 |
| Monocytes LPS | 7.0 | Colon | 6.8 |
| Macrophages rest | 12.2 | Lung | 9.5 |
| Macrophages LPS | 10.9 | Thymus | 14.3 |
| HUVEC none | 7.0 | Kidney | 28.9 |
| HUVEC starved | 17.7 | | |

Panel 1.3D Summary: Ag3060 Results from one experiment with this gene are not included. The amp plot indicates that there were experimental difficulties with this run (data not shown).

Panel 4D Summary: Ag3060 High expression of the NOV95 transcript (CTs=26.3-26.9) is found in activated B cells and B cell lymphoma (Ramos). B cells generate antibody response and lead to activation of T cell mediated response as antigen presenting cells and are central to the function of the immune response. Therefore, targeting of this gene product with a small molecule drug therapeutic may modulate the functions of B cells and lead to the improvement of symptoms of autoimmune diseases such as lupus erythematosus, rheumatoid arthritis, hyperglobulinemia and other B cell disorders.

In addition, moderate expression of this gene is also found in a wide range of cell types of significance in the immune response in health and diseases. This suggests the broader involvement of the protein encoded by this gene in many inflammatory and autoimmune diseases.

NOV96a, NOV96b, and NOV96c

Expression of gene NOV96a and full length clones NOV96b and NOV96c was assessed using the primer-probe set Ag4532, described in Table CKA. Results of the RTQ-PCR runs are shown in Table CKB.

TABLE CKA
Probe Name Ag4532

| | | | Start | |
|---|---|---|---|---|
| Primers | Sequences | Length | Position | SEQ ID NO: |
| Forward | 5′-actcacctctctcctccatcat-3′ | 22 | 626 | 1372 |
| Probe | TET-5′-cgttacactgttgccctcaccctgat-3′-TAMRA | 26 | 660 | 1373 |
| Reverse | 5′-agggaatgaagtagccagtgtt-3′ | 22 | 687 | 1374 |

TABLE CKB
General_screening_panel_v1.4

| | Rel. | | Rel. |
|---|---|---|---|
| | Exp. | | Exp. |
| | (%) | | (%) |
| | Ag4532, | | Ag4532, |
| | Run | | Run |
| Tissue Name | 222735297 | Tissue Name | 222735297 |
| Adipose | 2.0 | Renal ca. TK-10 | 31.9 |
| Melanoma* | 3.6 | Bladder | 3.7 |
| Hs688(A).T | | | |
| Melanoma* | 5.0 | Gastric ca. (liver met.) | 26.6 |
| Hs688(B).T | | NCI-N87 | |
| Melanoma* M14 | 18.2 | Gastric ca. KATO III | 33.7 |
| Melanoma* | 5.4 | Colon ca. SW-948 | 15.0 |
| LOXIMVI | | | |
| Melanoma* SK- | 2.7 | Colon ca. SW480 | 31.2 |
| MEL-5 | | | |
| Squamous cell | 6.3 | Colon ca.* (SW480 | 18.4 |
| carcinoma SCC-4 | | met) SW620 | |
| Testis Pool | 1.0 | Colon ca. HT29 | 4.2 |
| Prostate ca.* (bone | 7.3 | Colon ca. HCT-116 | 20.6 |
| met) PC-3 | | | |
| Prostate Pool | 1.5 | Colon ca. CaCo-2 | 17.1 |
| Placenta | 9.0 | Colon cancer tissue | 20.4 |
| Uterus Pool | 0.8 | Colon ca. SW1116 | 6.0 |
| Ovarian ca. | 10.7 | Colon ca. Colo-205 | 12.3 |
| OVCAR-3 | | | |
| Ovarian ca. | 2.5 | Colon ca. SW-48 | 13.1 |
| SK-OV-3 | | | |
| Ovarian ca. | 9.0 | Colon Pool | 3.4 |
| OVCAR-4 | | | |
| Ovarian ca. | 29.5 | Small Intestine Pool | 2.7 |
| OVCAR-5 | | | |
| Ovarian ca. | 4.0 | Stomach Pool | 1.1 |
| IGROV-1 | | | |
| Ovarian ca. | 14.6 | Bone Marrow Pool | 1.3 |
| OVCAR-8 | | | |
| Ovary | 1.7 | Fetal Heart | 1.7 |
| Breast ca. MCF-7 | 17.1 | Heart Pool | 2.1 |
| Breast ca. MDA- | 20.0 | Lymph Node Pool | 2.7 |
| MB-231 | | | |
| Breast ca. BT 549 | 32.3 | Fetal Skeletal Muscle | 0.7 |
| Breast ca. T47D | 68.8 | Skeletal Muscle Pool | 1.2 |
| Breast ca. MDA-N | 13.5 | Spleen Pool | 5.2 |
| Breast Pool | 2.3 | Thymus Pool | 4.0 |
| Trachea | 7.3 | CNS cancer | 47.3 |
| | | (glio/astro) | |
| | | U87-MG | |
| Lung | 0.2 | CNS cancer | 4.0 |
| | | (glio/astro) | |
| | | U-118-MG | |
| Fetal Lung | 8.4 | CNS cancer | 1.4 |
| | | (neuro; met) | |
| | | SK-N-AS | |
| Lung ca. NCI-N417 | 1.0 | CNS cancer | 13.6 |
| | | (astro) | |
| | | SF-539 | |
| Lung ca. LX-1 | 21.2 | CNS cancer (astro) | 16.5 |
| | | SNB-75 | |
| Lung ca. NCI-H146 | 0.2 | CNS cancer (glio) | 4.4 |
| | | SNB-19 | |
| Lung ca. SHP-77 | 13.8 | CNS cancer (glio) | 9.3 |
| | | SF-295 | |
| Lung ca. A549 | 44.4 | Brain (Amygdala) | 0.5 |
| | | Pool | |
| Lung ca. NCI-H526 | 3.1 | Brain (cerebellum) | 4.2 |
| Lung ca. NCI-H23 | 19.5 | Brain (fetal) | 3.8 |
| Lung ca. NCI-H460 | 7.5 | Brain (Hippocampus) | 1.1 |
| | | Pool | |
| Lung ca. HOP-62 | 17.4 | Cerebral Cortex Pool | 0.5 |
| Lung ca. NCI-H522 | 26.6 | Brain | 1.1 |
| | | (Substantia nigra) | |
| | | Pool | |
| Liver | 23.3 | Brain (Thalamus) Pool | 1.3 |
| Fetal Liver | 34.2 | Brain (whole) | 1.3 |
| Liver ca. HepG2 | 32.8 | Spinal Cord Pool | 1.3 |
| Kidney Pool | 4.6 | Adrenal Gland | 2.8 |
| Fetal Kidney | 3.8 | Pituitary gland Pool | 0.6 |
| Renal ca. 786-0 | 9.3 | Salivary Gland | 6.3 |
| Renal ca. A498 | 10.2 | Thyroid (female) | 6.0 |
| Renal ca. ACHN | 100.0 | Pancreatic ca. | 32.1 |
| | | CAPAN2 | |
| Renal ca. UO-31 | 22.2 | Pancreas Pool | 2.4 |

General_screening_panel_v1.4 Summary: Ag4532 The expression of the NOV96a gene appears to be highest in a sample derived from a renal cancer cell line (ACHN)(CT=26.4). In addition, there is substantial expression associated with other renal cancer cell lines as well as gastric cancer cell lines, colon cancer cell lines, lung cancer cell lines, and breast cancer cell lines. Thus, the expression of this gene could be used to distinguish ACHN cells from other samples in this panel. Moreover, therapeutic modulation of this gene, through the use of small molecule drugs, protein therapeutics or antibodies might be of benefit in the treatment of kidney, gastric, colon, lung or breast cancer.

Among metabolic tissues, this gene has low-to-moderate levels of expression in adipose, liver, heart, skeletal muscle, adrenal, pituitary, thyroid, and pancreas. Thus, this gene product may be a small molecule target for the treatment of endocrine and metabolic diseases, including obesity and Types 1 and 2 diabetes. The direction of therapeutic modulation for this gene product would, of necessity, be tissue- or organ-specific. The consequences of altered lactate/monocarboxylate/ketone body transport would differ dramatically between tissues.

In addition, this gene, a monocarboxylate transporter homolog, is expressed at low to moderate levels in all CNS regions examined. The monocarboxylate transporters have been implicated in post-ischemic neuronal loss in stroke, such that blockade of these transporters increase stroke-related damage. Thus, this gene is an excellent drug target, such that increasing its activity may decrease postischemic damage in stroke/cerebral infarct.

References:

Schurr A, Payne R S, Miller J J, Tseng M T, Rigor B M. Blockade of lactate transport exacerbates delayed neuronal damage in a rat model of cerebral ischemia. Brain Res Mar. 23, 2001;895(1-2):268-72

Studies over the past decade have demonstrated that lactate is produced aerobically during brain activation and it has been suggested to be an obligatory aerobic energy substrate postischemia. It has been also hypothesized, based on in vitro studies, that lactate, produced by glia in large amounts during activation and/or ischemia/hypoxia, is transported via specific glial and neuronal monocarboxylate transporters into neurons for aerobic utilization. To test the role of lactate as an aerobic energy substrate postischemia in vivo, we employed the cardiac-arrest-induced transient global cerebral ischemia (TGI) rat model and the monocarboxylate transporter inhibitor alpha-cyano-4-hydroxycinnamate (4-CIN). Once 4-CIN was establish to cross the blood—brain barrier, rats were treated with the inhibitor 60 min prior to a 5-min TGI. These rats exhibited a significantly greater degree of delayed neuronal damage in the hippocampus than control, untreated rats, as measured 7 days post-TGI. We concluded that intra-ischemically-accumulated lactate is utilized aerobically as the main energy substrate immediately postischemia. Blockade of lactate transport into neurons prevents its utilization and, consequently, exacerbates delayed ischemic neuronal damage.

NOV97c and NOV97d

Expression of gene NOV97c and variant NOV97d was assessed using the primer-probe set Ag3697, described in Table CLA. Results of the RTQ-PCR runs are shown in Table CLB.

TABLE CLA
Probe Name Ag3697

| | | | Start | |
|---|---|---|---|---|
| Primers | Sequences | Length | Position | SEQ ID NO: |
| Forward | 5′-catctggattgacactggaatt-3′ | 22 | 585 | 1375 |
| Probe | TET-5′-actcccgggagtggatcacccat-3′-TAMRA | 23 | 608 | 1376 |
| Reverse | 5′-aatcttattggcagtccagatg-3′ | 22 | 639 | 1377 |

TABLE CLB
General_screening panel_v1.4

| | Rel. | | Rel. |
|---|---|---|---|
| | Exp. | | Exp. |
| | (%) | | (%) |
| | Ag3697, | | Ag3697, |
| | Run | | Run |
| Tissue Name | 218253566 | Tissue Name | 218253566 |
| Adipose | 0.0 | Renal ca. TK-10 | 0.0 |
| Melanoma* | 0.0 | Bladder | 0.0 |
| Hs688(A).T | | | |
| Melanoma* | 0.3 | Gastric ca. (liver met.) | 0.0 |
| Hs688(B).T | | NCI-N87 | |
| Melanoma* M14 | 0.0 | Gastric ca. KATO III | 0.0 |
| Melanoma* | 0.0 | Colon ca. SW-948 | 1.0 |
| LOXIMVI | | | |
| Melanoma* SK- | 0.0 | Colon ca. SW480 | 8.2 |
| MEL-5 | | | |
| Squamous cell | 0.9 | Colon ca* (SW480 | 6.9 |
| carcinoma SCC-4 | | met) SW620 | |
| Testis Pool | 100.0 | Colon ca. HT29 | 0.0 |
| Prostate ca.* (bone | 0.3 | Colon ca. HCT-116 | 0.8 |
| met) PC-3 | | | |
| Prostate Pool | 0.0 | Colon ca. CaCo-2 | 0.3 |
| Placenta | 7.8 | Colon cancer tissue | 0.0 |
| Uterus Pool | 0.0 | Colon ca. SW1116 | 0.0 |
| Ovarian ca. | 0.6 | Colon ca. Colo-205 | 0.0 |
| OVCAR-3 | | | |
| Ovarian ca. | 1.3 | Colon ca. SW-48 | 0.0 |
| SK-OV-3 | | | |
| Ovarian ca. | 4.1 | Colon Pool | 0.7 |
| OVCAR-4 | | | |
| Ovarian ca. | 0.0 | Small Intestine Pool | 0.0 |
| OVCAR-5 | | | |
| Ovarian ca. | 0.8 | Stomach Pool | 0.0 |
| IGROV-1 | | | |
| Ovarian ca. | 2.0 | Bone Marrow Pool | 0.0 |
| OVCAR-8 | | | |
| Ovary | 0.2 | Fetal Heart | 0.0 |
| Breast ca. MCF-7 | 0.5 | Heart Pool | 0.7 |
| Breast ca. MDA- | 1.2 | Lymph Node Pool | 0.2 |
| MB-231 | | | |
| Breast ca. BT 549 | 0.4 | Fetal Skeletal Muscle | 4.5 |
| Breast ca. T47D | 0.0 | Skeletal Muscle Pool | 0.7 |
| Breast ca. MDA-N | 0.0 | Spleen Pool | 1.0 |
| Breast Pool | 0.3 | Thymus Pool | 1.8 |
| Trachea | 1.4 | CNS cancer | 1.1 |
| | | (glio/astro) | |
| | | U87-MG | |
| Lung | 0.9 | CNS cancer | 4.7 |
| | | (glio/astro) | |
| | | U-118-MG | |
| Fetal Lung | 1.2 | CNS cancer | 0.2 |
| | | (neuro; met) SK-N-AS | |
| Lung ca. NCI-N417 | 0.0 | CBS cancer (astro) | 0.0 |
| | | SF-539 | |
| Lung ca. LX-1 | 2.5 | CNS cancer (astro) | 1.7 |
| | | SNB-75 | |
| Lung ca. NCI-H146 | 1.4 | CNS cancer (glio) | 0.0 |
| | | SNB-19 | |
| Lung ca. SHP-77 | 28.9 | CNS cancer (glio) | 0.9 |
| | | SF-295 | |
| Lung ca. A549 | 0.5 | Brain | 0.0 |
| | | (Amygdala) Pool | |
| Lung ca. NCI-H526 | 0.0 | Brain (cerebellum) | 0.2 |
| Lung ca. NCI-H23 | 0.8 | Brain (fetal) | 0.0 |
| Lung ca. NCI-H460 | 0.7 | Brain (Hippocampus) | 0.0 |
| | | Pool | |
| Lung ca. HOP-62 | 1.4 | Cerebral Cortex Pool | 0.5 |
| Lung ca. NCI-H522 | 0.0 | Brain | 0.3 |
| | | (Substantia nigra) | |
| | | Pool | |
| Liver | 0.0 | Brain (Thalamus) Pool | 1.6 |
| Fetal Liver | 0.5 | Brain (whole) | 0.7 |
| Liver ca. HepG2 | 0.3 | Spinal Cord Pool | 0.3 |
| Kidney Pool | 0.0 | Adrenal Gland | 0.7 |
| Fetal Kidney | 0.0 | Pituitary gland Pool | 1.1 |
| Renal ca. 786-0 | 0.0 | Salivary Gland | 0.0 |
| Renal ca. A498 | 0.0 | Thyroid (female) | 0.0 |
| Renal ca. ACHN | 0.3 | Pancreatic ca. | 0.4 |
| | | CAPAN2 | |
| Renal ca. UO-31 | 4.1 | Pancreas Pool | 2.5 |

CNS_neurodegeneration_v1.0 Summary: Ag3697 Expression of this gene is low/undetectable (CTs>35) across all of the samples on this panel (data not shown).

General_screening_panel_v1.4 Summary: Ag3697 Expression of this gene is highest in and almost exclusive to testis (CT=30.7). Therefore, expression of this gene could be used to distinguish testis from the other samples on this panel. Moreover, therapeutic modulation of the activity of this gene or its protein product using protein therapeutics, antibodies or small molecule drugs could be of benefit in the treatment of infertility.

Panel 4.1D Summary: Ag3697 Expression of this gene is low/undetectable (CTs>35) across all of the samples on this panel (data not shown).

NOV98: AGRIN

Expression of gene NOV98 was assessed using the primer-probe set Ag3974, described in Table CMA. Results of the RTQ-PCR runs are shown in Tables CMB, CMC and CMD.

TABLE CMA
Probe Name Ag3974

| | | | Start | |
|---|---|---|---|---|
| Primers | Sequences | Length | Position | SEQ ID NO: |
| Forward | 5′-gacaccaggatcttctttgtga-3′ | 22 | 274 | 1378 |
| Probe | TET-5′-catacctgtggccagcccacaag-3′-TAMRA | 23 | 308 | 1379 |
| Reverse | 5′-gagttgagcatcagctcgtt-3′ | 20 | 331 | 1380 |

TABLE CMB
CNS_neurodegeneration_v1.0

| | Rel. | | Rel. |
|---|---|---|---|
| | Exp. | | Exp. |
| | (%) | | (%) |
| | Ag3974, | | Ag3974, |
| | Run | | Run |
| Tissue Name | 212348647 | Tissue Name | 212348647 |
| AD 1 Hippo | 28.7 | Control (Path) 3 | 15.4 |
| | | Temporal Ctx | |
| AD 2 Hippo | 36.3 | Control (Path) 4 | 46.0 |
| | | Temporal Ctx | |
| AD 3 Hippo | 19.2 | AD 1 Occipital Ctx | 28.9 |
| AD 4 Hippo | 21.8 | AD 2 Occipital Ctx | 0.0 |
| | | (Missing) | |
| AD 5 Hippo | 80.7 | AD 3 Occipital Ctx | 19.5 |
| AD 6 Hippo | 42.6 | AD 4 Occipital Ctx | 21.0 |
| Control 2 Hippo | 42.3 | AD 5 Occipital Ctx | 47.6 |
| Control 4 Hippo | 34.9 | AD 6 Occipital Ctx | 14.7 |
| Control (Path) | 12.4 | Control 1 | 20.4 |
| 3 Hippo | | Occipital Ctx | |
| AD 1 Temporal Ctx | 32.1 | Control 2 | 55.1 |
| | | Occipital Ctx | |
| AD 2 Temporal Ctx | 31.2 | Control 3 | 22.5 |
| | | Occipital Ctx | |
| AD 3 Temporal Ctx | 20.2 | Control 4 | 22.2 |
| | | Occipital Ctx | |
| AD 4 Temporal Ctx | 24.0 | Control (Path) 1 | 71.2 |
| | | Occipital Ctx | |
| AD 5 Inf Temporal | 100.0 | Control (Path) 2 | 17.4 |
| Ctx | | Occipital Ctx | |
| AD 5 Sup Temporal | 58.6 | Control (Path) 3 | 15.4 |
| Ctx | | Occipital Ctx | |
| AD 6 Inf Temporal | 40.6 | Control (Path) 4 | 38.7 |
| Ctx | | Occipital Ctx | |
| AD 6 Sup Temporal | 35.1 | Control 1 Parietal | 18.2 |
| Ctx | | Ctx | |
| Control 1 Temporal | 19.8 | Control 2 Parietal | 67.4 |
| Ctx | | Ctx | |
| Control 2 Temporal | 48.3 | Control 3 Parietal | 21.2 |
| Ctx | | Ctx | |
| Control 3 Temporal | 17.8 | Control (Path) 1 | 51.4 |
| Ctx | | Parietal Ctx | |
| Control 3 Temporal | 25.0 | Control (Path) 2 | 32.3 |
| Ctx | | Parietal Ctx | |
| Control (Path) 1 | 63.7 | Control (Path) 3 | 11.9 |
| Temporal Ctx | | Parietal Ctx | |
| Control (Path) 2 | 43.5 | Control (Path) 4 | 58.6 |
| Temporal Ctx | | Parietal Ctx | |

TABLE CMC
General_screening_panel_v1.4

| | Rel. | | Rel. |
|---|---|---|---|
| | Exp. | | Exp. |
| | (%) | | (%) |
| | Ag3974, | | Ag3974, |
| | Run | | Run |
| Tissue Name | 217508632 | Tissue Name | 217508632 |
| Adipose | 1.5 | Renal ca. TK-10 | 16.4 |
| Melanoma* | 3.2 | Bladder | 9.0 |
| Hs688(A).T | | | |
| Melanoma* | 4.2 | Gastric ca. (liver met.) | 80.7 |
| Hs688(B).T | | NCI-N87 | |
| Melanoma* M14 | 6.4 | Gastric ca. KATO III | 17.7 |
| Melanoma* | 4.0 | Colon ca. SW-948 | 7.8 |
| LOXIMVI | | | |
| Melanoma* SK- | 4.2 | Colon ca. SW480 | 32.3 |
| MEL-5 | | | |
| Squamous cell | 8.4 | Colon ca.* (SW480 | 4.6 |
| carcinoma SCC-4 | | met) SW620 | |
| Testis Pool | 1.1 | Colon ca. HT29 | 30.6 |
| Prostate ca.* (bone | 24.8 | Colon ca. HCT-116 | 5.8 |
| met) PC-3 | | | |
| Prostate Pool | 0.8 | Colon ca. CaCo-2 | 10.4 |
| Placenta | 1.3 | Colon cancer tissue | 10.0 |
| Uterus Pool | 0.4 | Colon ca. SW1116 | 3.6 |
| Ovarian ca. | 66.9 | Colon ca. Colo-205 | 1.5 |
| OVCAR-3 | | | |
| Ovarian ca. | 36.3 | Colon ca. SW-48 | 0.7 |
| SK-OV-3 | | | |
| Ovarian ca. | 12.7 | Colon Pool | 1.3 |
| OVCAR-4 | | | |
| Ovarian ca. | 44.4 | Small Intestine Pool | 1.0 |
| OVCAR-5 | | | |
| Ovarian ca. | 27.7 | Stomach Pool | 1.2 |
| IGROV-1 | | | |
| Ovarian ca. | 14.9 | Bone Marrow Pool | 0.5 |
| OVCAR-8 | | | |
| Ovary | 1.9 | Fetal Heart | 1.0 |
| Breast ca. MCF-7 | 9.7 | Heart Pool | 0.8 |
| Breast ca. MDA- | 31.2 | Lymph Node Pool | 2.0 |
| MB-231 | | | |
| Breast ca. BT 549 | 10.1 | Fetal Skeletal Muscle | 0.5 |
| Breast ca. T47D | 100.0 | Skeletal Muscle Pool | 0.5 |
| Breast ca. MDA-N | 4.2 | Spleen Pool | 0.7 |
| Breast Pool | 1.6 | Thymus Pool | 2.2 |
| Trachea | 2.6 | CNS cancer | 6.0 |
| | | (glio/astro) | |
| | | U87-MG | |
| Lung | 0.1 | CNS cancer | 11.2 |
| | | (glio/astro) | |
| | | U-118-MG | |
| Fetal Lung | 8.3 | CNS cancer | 0.9 |
| | | (neuro; met) | |
| | | SK-N-AS | |
| Lung ca. NCI-N417 | 0.7 | CNS cancer | 5.0 |
| | | (astro) | |
| | | SF-539 | |
| Lung ca. LX-1 | 11.0 | CNS cancer (astro) | 32.3 |
| | | SNB-75 | |
| Lung ca. NCI-H146 | 0.1 | CNS cancer (glio) | 20.2 |
| | | SNB-19 | |
| Lung ca. SHP-77 | 0.8 | CNS cancer (glio) | 38.2 |
| | | SF-295 | |
| Lung ca. A549 | 10.4 | Brain (Amygdala) | 1.3 |
| | | Pool | |
| Lung ca. NCI-H526 | 1.6 | Brain (cerebellum) | 1.0 |
| Lung ca. NCI-H23 | 20.6 | Brain (fetal) | 2.8 |
| Lung ca. NCI-H460 | 9.3 | Brain (Hippocampus) | 0.9 |
| | | Pool | |
| Lung ca. HOP-62 | 23.0 | Cerebral Cortex Pool | 0.9 |
| Lung ca. NCI-H522 | 2.3 | Brain | 1.7 |
| | | (Substantia nigra) | |
| | | Pool | |
| Liver | 0.6 | Brain (Thalamus) Pool | 1.6 |
| Fetal Liver | 1.4 | Brain (whole) | 1.1 |
| Liver ca. HepG2 | 12.6 | Spinal Cord Pool | 1.4 |
| Kidney Pool | 2.5 | Adrenal Gland | 0.4 |
| Fetal Kidney | 4.6 | Pituitary gland Pool | 0.2 |
| Renal ca. 786-0 | 39.5 | Salivary Gland | 1.3 |
| Renal ca. A498 | 7.9 | Thyroid (female) | 3.7 |
| Renal ca. ACHN | 15.9 | Pancreatic ca. | 27.7 |
| | | CAPAN2 | |
| Renal ca. UO-31 | 38.7 | Pancreas Pool | 4.1 |

TABLE CMD
Panel 4.1D

| | Rel. | | Rel. |
|---|---|---|---|
| | Exp. | | Exp. |
| | (%) | | (%) |
| | Ag3974, | | Ag3974, |
| | Run | | Run |
| Tissue Name | 170739806 | Tissue Name | 170739806 |
| Secondary Th1 act | 1.2 | HUVEC IL-1beta | 18.9 |
| Secondary Th2 act | 8.0 | HUVEC IFN gamma | 16.7 |
| Secondary Tr1 act | 3.5 | HUVEC TNF alpha + | 34.9 |
| | | IFN gamma | |
| Secondary Th1 rest | 0.7 | HUVEC TNF alpha + | 31.4 |
| | | IL4 | |
| Secondary Th2 rest | 0.2 | HUVEC IL-11 | 13.9 |
| Secondary Tr1 rest | 1.2 | Lung Microvascular | 100.0 |
| | | EC none | |
| Primary Th1 act | 3.2 | Lung Microvascular | 97.9 |
| | | EC TNFalpha + | |
| | | IL-1beta | |
| Primary Th2 act | 2.0 | Microvascular Dermal | 48.3 |
| | | EC none | |
| Primary Tr1 act | 2.9 | Microvasular Dermal | 47.0 |
| | | EC TNFalpha + | |
| | | IL-1beta | |
| Primary Th1 rest | 0.4 | Bronchial epithelium | 90.1 |
| | | TNFalpha + IL1beta | |
| Primary Th2 rest | 0.2 | Small airway | 32.5 |
| | | epithelium none | |
| Primary Tr1 rest | 0.3 | Small airway | 93.3 |
| | | epithelium | |
| | | TNFalpha + IL-1beta | |
| CD45RA CD4 | 22.7 | Coronery artery | 28.5 |
| lymphocyte act | | SMC rest | |
| CD45RO CD4 | 5.5 | Coronery artery | 28.7 |
| lymphocyte act | | SMC TNFalpha + | |
| | | IL-1beta | |
| CD8 lymphocyte act | 3.3 | Astrocytes rest | 55.1 |
| Secondary CD8 | 3.3 | Astrocytes | 66.4 |
| lymphocyte rest | | TNFalpha + | |
| | | IL-1beta | |
| Secondary CD8 | 3.5 | KU-812 (Basophil) | 1.9 |
| lynphocyte act | | rest | |
| CD4 lymphocyte | 0.1 | KU-812 (Basophil) | 2.8 |
| none | | PMA/ionomycin | |
| 2ry Th1/Th2/ | 0.4 | CCD1106 | 82.4 |
| Tr1_anti- | | (Keratinocytes) | |
| CD95 CH11 | | none | |
| LAK cells rest | 6.4 | CCD1106 | 72.7 |
| | | (Keratinocytes) | |
| | | TNFalpha + IL-1beta | |
| LAK cells IL-2 | 1.7 | Liver cirrhosis | 14.4 |
| LAK cells IL-2 + | 1.8 | NCI-H292 none | 54.0 |
| IL-12 | | | |
| LAK cells IL-2 + | 1.1 | NCI-H292 IL-4 | 78.5 |
| IFN gamma | | | |
| LAK cells IL-2 + | 1.6 | NCI-H292 IL-9 | 79.6 |
| IL-18 | | | |
| LAK cells | 4.6 | NCI-H292 IL-13 | 59.9 |
| PMA/ionomycin | | | |
| NK Cells IL-2 rest | 1.9 | NCI-H292 IFN gamma | 71.7 |
| Two Way MLR | 12.4 | HPAEC none | 21.3 |
| 3 day | | | |
| Two Way MLR | 5.3 | HPAEC TNF alpha + | 45.4 |
| 5 day | | IL-1beta | |
| Two Way MLR | 4.0 | Lung fibroblast none | 29.3 |
| 7 day | | | |
| PBMC rest | 0.6 | Lung fibroblast | 87.7 |
| | | TNF alpha + | |
| | | IL-1beta | |
| PBMC PWM | 4.9 | Lung fibroblast IL-4 | 23.3 |
| PBMC PHA-L | 3.4 | Lung fibroblast IL-9 | 30.4 |
| Ramos (B cell) none | 0.4 | Lung fibroblast IL-13 | 36.6 |
| Ramos (B cell) | 0.2 | Lung fibroblast IFN | 29.7 |
| ionomycin | | gamma | |
| B lymphocytes | 3.0 | Dermal fibroblast | 27.2 |
| PMW | | CCD1070 rest | |
| B lymphocytes | 3.7 | Dermal fibroblast | 20.6 |
| CD40L and IL-4 | | CCD1070 TNF alpha | |
| EOL-1 dbAMP | 3.1 | Dermal fibroblast | 22.4 |
| | | CCD1070 IL-1beta | |
| EOL-1 dbcAMP | 8.0 | Dermal fibroblast IFN | 10.3 |
| PMA/ionomycin | | gamma | |
| Dendritic cells none | 9.0 | Dermal fibroblast IL-4 | 80 |
| Dendritic cells LPS | 32.8 | Dermal Fibroblasts | 6.3 |
| | | rest | |
| Dendritic cells anti- | 8.8 | Neutrophils TNFa + | 0.9 |
| CD40 | | LPS | |
| Monocytes rest | 1.4 | Neutrophils rest | 1.0 |
| Monocytes LPS | 81.2 | Colon | 5.8 |
| Macrophages rest | 9.7 | Lung | 23.3 |
| Macrophages LPS | 43.8 | Thymus | 7.3 |
| HUVEC none | 12.6 | Kidney | 33.2 |
| HUVEC starved | 25.2 | | |

CNS_neurodegeneration_v1.0 Summary: Ag3974 This panel does not show differential expression of the NOV98 gene in Alzheimer's disease. However, this expression profile confirms the presence of this gene in the brain. Please see Panel 1.3D for discussion of utility of this gene in the central nervous system.

General_screening_panel_v1.4 Summary: Ag3974 The expression of the NOV98 gene appears to be highest in a sample derived from a breast cancer cell line (T47D) (CT=22.5). In addition, there appears to be substantial expression in other samples derived from breast cancer cell lines, ovarian cancer cell lines, kidney cancer cell lines, lung cancer cell lines, colon cancer cell lines and brain cancer cell lines. Thus, the expression of this gene could be used to distinguish T47D cells from other samples in the panel. Moreover, therapeutic modulation of this gene, through the use of small molecule drugs, protein therapeutics, or antibodies could be of benefit in the treatment of breast, ovarian, kidney, lung, colon or brain cancer.

Among metabolic tissues, this gene has low-to-moderate levels of expression in adrenal, pituitary, adult and fetal heart, adult and fetal liver, adult and fetal skeletal muscle, and adipose. This gene product has high levels of expression (CT values=27) in pancreas and thyroid. Thus, this gene product may be important for the pathogenesis, diagnosis, and/or treatment of metabolic and endocrine diseases, including obesity, Types 1 and 2 diabetes and thyroidopathies. It has recently been reported that an agrin minigene rescued dystrophic symptoms in a mouse model of muscular dystrophy. Therefore, this gene product may also be used as a treatment or cure for congenital muscular dystrophies.

This gene is also expressed at moderate to high levels in all regions of the CNS. This molecule is a homolog of agrin, which has been implicated in the formation of senile plaques in Alzheimer's disease and in the acetylcholine synapse/neuromuscular junction. This gene is therefore an excellent drug target in AD or in any disease involving the neuromuscular junction or the acetylchpoline system.

References:

Moll J, Barzaghi P, Lin S, Bezakova G, Lochmuller H, Engvall E, Muller U, Ruegg M A. An agrin minigene rescues dystrophic symptoms in a mouse model for congenital muscular dystrophy. Nature. Sep. 20, 2001;413(6853):302-7.

Congenital muscular dystrophy is a heterogeneous and severe, progressive muscle-wasting disease that frequently leads to death in early childhood. Most cases of congenital muscular dystrophy are caused by mutations in LAMA2, the gene encoding the alpha2 chain of the main laminin isoforms expressed by muscle fibres. Muscle fibre deterioration in this disease is thought to be caused by the failure to form the primary laminin scaffold, which is necessary for basement membrane structure, and the missing interaction between muscle basement membrane and the dystrophin-glycoprotein complex (DGC) or the integrins. With the aim to restore muscle function in a mouse model for this disease, we have designed a minigene of agrin, a protein known for its role in the formation of the neuromuscular junction. Here we show that this mini-agrin-which binds to basement membrane and to alpha-dystroglycan, a member of the DGC-amends muscle pathology by a mechanism that includes agrin-mediated stabilization of alpha-dystroglycan and the laminin alpha5 chain. Our data provides in vivo evidence that a non-homologous protein in combination with rational protein design can be used to devise therapeutic tools that may restore muscle function in human muscular dystrophies.

PMID: 11565031

Liyanage Y, Hoch W, Beeson D, Vincent A. The agrin/muscle-specific kinase pathway: New targets for autoimmune and genetic disorders at the neuromuscular junction. Muscle Nerve January 2002;25(1):4-16

The increasing understanding of the structural complexity of the neuromuscular junction (NMJ), and the processes that are important in its development, suggests many possible new disease targets. Here, we summarize briefly the genetic and autoimmune disorders that affect neuromuscular transmission, and the identified targets, including new evidence that antibodies to muscle-specific receptor tyrosine kinase (MuSK) are involved in the pathogenesis of acetylcholine receptor (AChR) antibody-negative myasthenia gravis. We then review the development of the NMJ, focusing on the important roles of nerve-derived agrin and MuSK in clustering of AChRs and other essential components of the NMJ.

van Horssen J, Otte-Holler I, David G, Maat-Schieman M L, van den Heuvel L P, Wesseling P, de Waal R M, Verbeek M M. Heparan sulfate proteoglycan expression in cerebrovascular amyloid beta deposits in Alzheimer's disease and hereditary cerebral hemorrhage with amyloidosis (Dutch) brains. Acta Neuropathol (Berl) December 2001;102(6):604-14

Cerebrovascular deposition of amyloid beta protein (A beta) is a characteristic lesion of Alzheimer's disease (AD) and hereditary cerebral hemorrhage with amyloidosis of the Dutch type (HCHWA-D). Besides A beta, several other proteins and proteoglycans accumulate in cerebral amyloid angiopathy (CAA). We have now analyzed the expression of the heparan sulfate proteoglycan (HSPG) subtypes agrin, perlecan, glypican-1, syndecans 1-3 and HS glycosaminoglycan (GAG) side chains in CAA in brains of patients with AD and HCHWA-D. Hereto, specific well-characterized antibodies directed against the core protein of these HSPGs and against the GAG side chains were used for immunostaining. Glypican-1 was abundantly expressed in CAA both in AD and HCHWA-D brains, whereas perlecan and syndecans-1 and -3 were absent in both. Colocalization of agrin with vascular A beta was clearly observed in CAA in HCHWA-D brains, but only in a minority of the AD cases. Conversely, syndecan-2 was frequently associated with vascular A beta in AD, but did not colocalize with vascular A beta deposits in HCHWA-D. The three different syndecans, agrin, glypican-1 and HS GAG, but not perlecan, were associated with the majority of senile plaques (SPs) in all brains. Our results suggest a role for agrin in the formation of SPs and of CAA in HCHWA-D, but not in the pathogenesis of CAA in AD. Both syndecan-2 and glypican, but not perlecan, may be involved in the formation of CAA. We conclude that specific HSPG species may be involved in the pathogenesis of CAA in both AD and HCHWA-D, and that the pathogenesis of CAA and SPs may differ with regard to the involvement of HSPG species.

Panel 4.1D Summary: Ag3974 The NOV98 gene is expressed at moderate levels (CT=29-32) in a wide range of cell types of significance in the immune response in health and disease. Therefore, targeting of this gene product with a small molecule drug or antibody therapeutic may modulate the functions of cells of the immune system as well as resident tissue cells and lead to improvement of the symptoms of patients suffering from autoimmune and inflammatory diseases such as COPD, emphysema, asthma, allergies, inflammatory bowel disease, lupus erythematosus, and arthritis, including osteoarthritis and rheumatoid arthritis. Based on its homology to agrin, this gene product may also be beneficial to the treatment of multiple sclerosis as suggested by the referene below.

References:

Liyanage Y, Hoch W, Beeson D, Vincent A. The agrin/muscle-specific kinase pathway: New targets for autoimmune and genetic disorders at the neuromuscular junction. Muscle Nerve January 2002;25(1):4-16

The increasing understanding of the structural complexity of the neuromuscular junction (NMJ), and the processes that are important in its development, suggests many possible new disease targets. Here, we summarize briefly the genetic and autoimmune disorders that affect neuromuscular transmission, and the identified targets, including new evidence that antibodies to muscle-specific receptor tyrosine kinase (MuSK) are involved in the pathogenesis of acetylcholine receptor (AChR) antibody-negative myasthenia gravis. We then review the development of the NMJ, focusing on the important roles of nerve-derived agrin and MuSK in clustering of AChRs and other essential components of the NMJ.

Example 3. SNP Analysis of NOVX Clones

SeqCalling™ Technology: cDNA was derived from various human samples representing multiple tissue types, normal and diseased states, physiological states, and developmental states from different donors. Samples were obtained as whole tissue, cell lines, primary cells or tissue cultured primary cells and cell lines. Cells and cell lines may have been treated with biological or chemical agents that regulate gene expression for example, growth factors, chemokines, steroids. The cDNA thus derived was then sequenced using CuraGen's proprietary SeqCalling technology. Sequence traces were evaluated manually and edited for corrections if appropriate. cDNA sequences from all samples were assembled with themselves and with public ESTs using bioinformatics programs to generate CuraGen's human SeqCalling database of SeqCalling assemblies. Each assembly contains one or more overlapping cDNA sequences derived from one or more human samples. Fragments and ESTs were included as components for an assembly when the extent of identity with another component of the assembly was at least 95% over 50 bp. Each assembly can represent a gene and/or its variants such as splice forms and/or single nucleotide polymorphisms (SNPs) and their combinations.

Variant sequences are included in this application. A variant sequence can include a single nucleotide polymorphism (SNP). A SNP can, in some instances, be referred to as a “cSNP” to denote that the nucleotide sequence containing the SNP originates as a cDNA. A SNP can arise in several ways. For example, a SNP may be due to a substitution of one nucleotide for another at the polymorphic site. Such a substitution can be either a transition or a transversion. A SNP can also arise from a deletion of a nucleotide or an insertion of a nucleotide, relative to a reference allele. In this case, the polymorphic site is a site at which one allele bears a gap with respect to a particular nucleotide in another allele. SNPs occurring within genes may result in an alteration of the amino acid encoded by the gene at the position of the SNP. Intragenic SNPs may also be silent, however, in the case that a codon including a SNP encodes the same amino acid as a result of the redundancy of the genetic code. SNPs occurring outside the region of a gene, or in an intron within a gene, do not result in changes in any amino acid sequence of a protein but may result in altered regulation of the expression pattern for example, alteration in temporal expression, physiological response regulation, cell type expression regulation, intensity of expression, stability of transcribed message.

Method of novel SNP Identification: SNPs are identified by analyzing sequence assemblies using CuraGen's proprietary SNPTool algorithm. SNPTool identifies variation in assemblies with the following criteria: SNPs are not analyzed within 10 base pairs on both ends of an alignment; Window size (number of bases in a view) is 10; The allowed number of mismatches in a window is 2; Minimum SNP base quality (PHRED score) is 23; Minimum number of changes to score an SNP is 2/assembly position. SNPTool analyzes the assembly and displays SNP positions, associated individual variant sequences in the assembly, the depth of the assembly at that given position, the putative assembly allele frequency, and the SNP sequence variation. Sequence traces are then selected and brought into view for manual validation. The consensus assembly sequence is imported into CuraTools along with variant sequence changes to identify potential amino acid changes resulting from the SNP sequence variation. Comprehensive SNP data analysis is then exported into the SNPCalling database.

Method of novel SNP Confirmation: SNPs are confirmed employing a validated method know as Pyrosequencing (Pyrosequencing, Westborough, Mass.). Detailed protocols for Pyrosequencing can be found in: Alderborn et al. Determination of Single Nucleotide Polymorphisms by Real-time Pyrophosphate DNA Sequencing. (2000). Genome Research. 10, Issue 8,August. 1249-1265. In brief, Pyrosequencing is a real time primer extension process of genotyping. This protocol takes double-stranded, biotinylated PCR products from genomic DNA samples and binds them to streptavidin beads. These beads are then denatured producing single stranded bound DNA. SNPs are characterized utilizing a technique based on an indirect bioluminometric assay of pyrophosphate (PPi) that is released from each dNTP upon DNA chain elongation. Following Klenow polymerase-mediated base incorporation, PPi is released and used as a substrate, together with adenosine 5′-phosphosulfate (APS), for ATP sulfurylase, which results in the formation of ATP. Subsequently, the ATP accomplishes the conversion of luciferin to its oxi-derivative by the action of luciferase. The ensuing light output becomes proportional to the number of added bases, up to about four bases. To allow processivity of the method dNTP excess is degraded by apyrase, which is also present in the starting reaction mixture, so that only dNTPs are added to the template during the sequencing. The process has been fully automated and adapted to a 96-well format, which allows rapid screening of large SNP panels. The DNA and protein sequences for the novel single nucleotide polymorphic variants are reported. Variants are reported individually but any combination of all or a select subset of variants are also included. In addition, the positions of the variant bases and the variant amino acid residues are underlined.

### Results

Variants are reported individually but any combination of all or a select subset of variants are also included as contemplated NOVX embodiments of the invention.

NOV1a SNP Data:

NOV1a has two SNP variants, whose variant positions for its nucleotide and amino acid sequences is numbered according to SEQ ID NOs:1 and 2, respectively. The nucleotide sequence of the NOV1a variant differs as shown in Table 101.

TABLE 101
cSNP and Coding Variants for NOV1a

| NT Position | Wild Type | | Amino Acid | Amino Acid |
|---|---|---|---|---|
| of cSNP | NT | Variant NT | position | Change |
| 393 | T | C | 130 | S −> P |
| 420 | T | C | 139 | W −> R |
| 431 | T | C | 142 | No change |
| 501 | C | T | 166 | L −> F |
| | | | | Putative |
| NT Position | Wild Type | | | Allele |
| of cSNP | NT | Variant NT | Depth | Frequency |
| 420 | T | C | 20 | 0.100 |

NOV1b SNP Data:

NOV1b has four SNP variants, whose variant positions for its nucleotide and amino acid sequences is numbered according to SEQ ID NOs:3 and 4, respectively. The nucleotide sequence of the NOV1b variant differs as shown in Table 102.

TABLE 102
cSNP and Coding Variants for NOV1b

| NT Position | Wild Type | | Amino Acid | Amino Acid |
|---|---|---|---|---|
| of cSNP | NT | Variant NT | position | Change |
| 393 | T | C | 130 | S −> P |
| 420 | T | C | 139 | W −> R |

NOV3a SNP Data:

NOV3a has four SNP variants, whose variant positions for its nucleotide and amino acid sequences is numbered according to SEQ ID NOs:11 and 12, respectively. The nucleotide sequence of the NOV3a variant differs as shown in Table 103.

TABLE 103
cSNP and Coding Variants for NOV3a

| NT Position | Wild Type | | Amino Acid | Amino Acid |
|---|---|---|---|---|
| of cSNP | NT | Variant NT | position | Change |
| 212 | T | C | 54 | C −> R |
| 439 | A | G | 149 | No Change |

NOV4a SNP Data:

NOV4a has four SNP variants, whose variant positions for its nucleotide and amino acid sequences is numbered according to SEQ ID NOs:17 and 18, respectively. The nucleotide sequence of the NOV4a variant differs as shown in Table 104.

TABLE 104
cSNP and Coding Variants for NOV4a

| NT Position | Wild Type | | Amino Acid | Amino Acid |
|---|---|---|---|---|
| of cSNP | NT | Variant NT | position | Change |
| 229 | G | A | 73 | No change |
| 390 | G | A | 127 | W −> End |
| 631 | G | C | 207 | Q −> H |

NOV5a SNP Data:

NOV5a has four SNP variants, whose variant positions for its nucleotide and amino acid sequences is numbered according to SEQ ID NOs:23 and 24, respectively. The nucleotide sequence of the NOV5a variant differs as shown in Table 105.

TABLE 105
cSNP and Coding Variants for NOV5a

| NT Position | Wild Type | | Amino Acid | Amino Acid |
|---|---|---|---|---|
| of cSNP | NT | Variant NT | position | Change |
| 79 | C | T | 19 | A −> V |
| 204 | T | C | 61 | No change |
| 658 | A | T | 212 | Q −> L |
| 884 | C | G | 287 | No change |
| 1149 | G | T | 376 | D −> Y |

NOV6 SNP Data:

NOV6 has four SNP variants, whose variant positions for its nucleotide and amino acid sequences numbered according to SEQ ID NOs:33 and 34, respectively. The nucleotide sequence of the NOV6 variant differs as shown in Table 106.

TABLE 106
cSNP and Coding Variants for NOV6b

| NT Position | Wild Type | | Amino Acid | Amino Acid |
|---|---|---|---|---|
| of cSNP | NT | Variant NT | position | Change |
| 543 | T | C | 156 | V −> A |
| 549 | T | C | 158 | I −> T |
| 660 | C | T | 195 | A −> V |
| 734 | A | G | 220 | T −> A |
| 782 | G | A | 236 | A −> T |

NOV7a SNP Data:

NOV7a has four SNP variants, whose variant positions for its nucleotide and amino acid sequences is numbered according to SEQ ID NOs:35 and 36, respectively. The nucleotide sequence of the NOV7a variant differs as shown in Table 107.

TABLE 107
cSNP and Coding Variants for NOV7a

| NT Position | Wild Type | | Amino Acid | Amino Acid |
|---|---|---|---|---|
| of cSNP | NT | Variant NT | position | Change |
| 168 | T | C | 24 | V −> G |
| 459 | C | T | 121 | A −> V |
| 815 | T | C | 240 | S −> P |
| 896 | A | G | N/A | No change |
| | | | | Putative |
| NT Position | Wild Type | | | Allele |
| of cSNP | NT | Variant NT | Depth | Frequency |
| 428 | G | A | 8 | 0.250 |

NOV7c SNP Data:

NOV7c has four SNP variants, whose variant positions for its nucleotide and amino acid sequences is numbered according to SEQ ID NOs:39 and 40, respectively. The nucleotide sequence of the NOV7c variant differs as shown in Table 108.

TABLE 108
cSNP and Coding Variants for NOV7c

| | | | | Putative |
|---|---|---|---|---|
| NT Position | Wild Type | | | Allele |
| of cSNP | NT | Variant NT | Depth | Frequency |
| 383 | C | T | 5 | 0.400 |

NOV7d SNP Data:

NOV7d has four SNP variants, whose variant positions for its nucleotide and amino acid sequences is numbered according to SEQ ID NOs:41 and 42, respectively. The nucleotide sequence of the NOV7d variant differs as shown in Table 109.

TABLE 109
cSNP and Coding Variants for NOV7d

| NT Position | Wild Type | | Amino Acid | Amino Acid |
|---|---|---|---|---|
| of cSNP | NT | Variant NT | position | Change |
| 260 | G | A | 86 | I −> E |

NOV7e SNP Data:

NOV7e has four SNP variants, whose variant positions for its nucleotide and amino acid sequences is numbered according to SEQ ID NOs:323 and 324, respectively. The nucleotide sequence of the NOV7d variant differs as shown in Table 110.

TABLE 110
cSNP and Coding Variants for NOV7e

| NT Position | Wild Type | | Amino Acid | Amino Acid |
|---|---|---|---|---|
| of cSNP | NT | Variant NT | position | Change |
| 304 | A | G | 102 | T −> A |

NOV9a SNP Data:

NOV9a has one SNP variant, whose variant position for its nucleotide and amino acid sequences is numbered according to SEQ ID NOs:45 and 46, respectively. The nucleotide sequence of the NOV9a variant differs as shown in Table 111.

TABLE 111
cSNP and Coding Variants for NOV9a

| NT Position | Wild Type | | Amino Acid | Amino Acid |
|---|---|---|---|---|
| of cSNP | NT | Variant NT | position | Change |
| 264 | G | A | N/A | No change |
| 391 | C | A | 8 | P −> T |
| 438 | G | A | 23 | No change |
| 550 | T | G | 61 | F −> V |
| 672 | C | T | 101 | No change |
| 1286 | T | C | 306 | L −> S |
| 1338 | G | A | 323 | No change |

NOV10 SNP Data:

NOV10 has one SNP variant, whose variant position for its nucleotide and amino acid sequences is numbered according to SEQ ID NOs:49 and 50, respectively. The nucleotide sequence of the NOV10 variant differs as shown in Table 112.

TABLE 112
cSNP and Coding Variants for NOV10

| | | | | Putative |
|---|---|---|---|---|
| NT Position | Wild Type | | | Allele |
| of cSNP | NT | Variant NT | Depth | Frequency |
| 335 | G | A | 10 | 0.400 |

NOV13b SNP Data:

NOV13b has one SNP variant, whose variant position for its nucleotide and amino acid sequences is numbered according to SEQ ID NOs:57 and 58, respectively. The nucleotide sequence of the NOV13b variant differs as shown in Table 113.

TABLE 113
cSNP and Coding Variants for NOV13b

| | | | | Putative |
|---|---|---|---|---|
| NT Position | Wild Type | | | Allele |
| of cSNP | NT | Variant NT | Depth | Frequency |
| 362 | G | A | 11 | 0.455 |

NOV15b SNP Data:

NOV15b has one SNP variant, whose variant position for its nucleotide and amino acid sequences is s numbered according to SEQ ID NOs:63 and 64, respectively. The nucleotide sequence of the NOV15b variant differs as shown in Table 114.

TABLE 114
cSNP and Coding Variants for NOV15b

| | | | | Putative |
|---|---|---|---|---|
| NT Position | Wild Type | | | Allele |
| of cSNP | NT | Variant NT | Depth | Frequency |
| 388 | T | G | 17 | 0.294 |

NOV16b SNP Data:

NOV16b has one SNP Variant, whose variant position for its nucleotide and amino acid sequences is s numbered according to SEQ ID NOs:67 and 68, respectively. The nucleotide sequence of the NOV16b variant differs as shown in Table 115.

TABLE 115
cSNP and Coding Variants for NOV16b

| | | | | Putative |
|---|---|---|---|---|
| NT Position | Wild Type | | | Allele |
| of cSNP | NT | Variant NT | Depth | Frequency |
| 463 | A | T | 16 | 0.125 |
| 465 | C | T | 16 | 0.125 |
| 535 | T | C | 15 | 0.133 |
| 735 | C | T | 12 | 0.167 |
| 814 | T | G | 11 | 0.182 |

NOV21a SNP Data:

NOV21a has one SNP variant, whose variant position for its nucleotide and amino acid sequences is numbered according to SEQ ID NOs:87 and 88, respectively. The nucleotide sequence of the NOV21a variant differs as shown in Table 116.

TABLE 116
cSNP and Coding Variants for NOV21a

| NT Position | Wild Type | | Amino Acid | Amino Acid |
|---|---|---|---|---|
| of cSNP | NT | Variant NT | position | Change |
| 121 | T | C | 41 | W−>R |
| 170 | T | C | 57 | V−>A |
| 364 | C | T | 122 | No change |
| 415 | G | A | 139 | E−>K |
| 535 | G | A | 182 | R−>H |
| 630 | G | A | 210 | No change |

NOV21b SNP Data:

NOV21b has one SNP variant, whose variant position for its nucleotide and amino acid sequences is numbered according to SEQ ID NOs:89 and 90, respectively. The nucleotide sequence of the NOV21b variant differs as shown in Table 117.

TABLE 117
cSNP and Coding Variants for NOV21b

| | | | | Putative |
|---|---|---|---|---|
| NT Position | Wild Type | | | Allele |
| of cSNP | NT | Variant NT | Depth | Frequency |
| 485 | G | A | 65 | 0.246 |
| 616 | G | A | 44 | 0.136 |
| 714 | G | A | 36 | 0.083 |

NOV22a SNP Data:

NOV22a has one SNP variant, whose variant position for its nucleotide and amino acid sequences is numbered according to SEQ ID NOs:91 and 92, respectively. The nucleotide sequence of the NOV22a variant differs as shown in Table 118.

TABLE 118
cSNP and Coding Variants for NOV22a

| NT Position | Wild Type | | Amino Acid | Amino Acid |
|---|---|---|---|---|
| of cSNP | NT | Variant NT | position | Change |
| 669 | A | G | 223 | No change |
| 725 | C | T | 242 | T−>M |

NOV22c SNP Data:

NOV22c has one SNP variant, whose variant position for its nucleotide and amino acid sequences is numbered according to SEQ ID NOs:95 and 96, respectively. The nucleotide sequence of the NOV22c variant differs as shown in Table 119.

TABLE 119
cSNP and Coding Variants for NOV22c

| | | | | Putative |
|---|---|---|---|---|
| NT Position | Wild Type | | | Allele |
| of cSNP | NT | Variant NT | Depth | Frequency |
| 85 | A | G | 30 | 0.067 |
| 288 | T | C | 31 | 0.065 |
| 484 | A | G | 37 | 0.054 |
| 540 | C | T | 29 | 0.241 |

NOV24a SNP Data:

NOV24a has one SNP variant, whose variant position for its nucleotide and amino acid sequence is numbered according to SEQ ID NOs:103 and 104, respectively. The nucleotide sequence of the NOV24a variant differs as shown in Table 120.

TABLE 120
cSNP and Coding Variants for NOV24a

| NT Position | Wild Type | | Amino Acid | Amino Acid |
|---|---|---|---|---|
| of cSNP | NT | Variant NT | position | Change |
| 539 | C | T | 511 | No change |

NOV24b SNP Data:

NOV24b has one SNP variant, whose variant position for its nucleotide and amino acid sequences is numbered according to SEQ ID NOs:105 and 106, respectively. The nucleotide sequence of the NOV24b variant differs as shown in Table 121.

TABLE 121
cSNP and Coding Variants for NOV24b

| NT Position | Wild Type | | Amino Acid | Amino Acid |
|---|---|---|---|---|
| of cSNP | NT | Variant NT | position | Change |
| 437 | A | G | 143 | N−>S |
| 664 | T | G | 219 | F−>V |
| 1150 | G | T | 381 | A−>S |
| 1210 | G | T | 401 | E−>End |
| 1770 | C | T | 587 | No change |
| 2011 | A | G | N/A | No change |

| | | | | Putative |
|---|---|---|---|---|
| NT Position | Wild Type | | | Allele |
| of cSNP | NT | Variant NT | Depth | Frequency |
| 329 | C | T | 11 | 0.364 |
| 491 | A | C | 13 | 0.154 |

NOV25 SNP Data:

NOV25 has one SNP variant, whose variant position for its nucleotide and amino acid sequences is s numbered according to SEQ ID NOs:107 and 108, respectively. The nucleotide sequence of the NOV25 variant differs as shown in Table 122.

TABLE 122
cSNP and Coding Variants for NOV25

| NT Position | Wild Type | | Amino Acid | Amino Acid |
|---|---|---|---|---|
| of cSNP | NT | Variant NT | position | Change |
| 221 | G | A | 54 | No change |
| 462 | C | T | 135 | L−>F |

NOV26a SNP Data:

NOV26a has one SNP variant, whose variant position for its nucleotide and amino acid sequences is numbered according to SEQ ID NOs:109 and 110, respectively. The nucleotide sequence of the NOV26a variant differs as shown in Table 123.

TABLE 123
cSNP and Coding Variants for NOV26a

| NT Position | Wild Type | | Amino Acid | Amino Acid |
|---|---|---|---|---|
| of cSNP | NT | Variant NT | position | Change |
| 67 | A | G | 1 | M−>V |
| 98 | C | T | 11 | P−>L |
| 128 | A | G | 21 | E−>G |
| 176 | A | G | 37 | A−>T |
| 233 | A | G | 56 | Q−>R |
| 243 | T | C | 59 | No change |
| 252 | A | G | 62 | No change |
| 260 | A | G | 65 | D−>G |
| 296 | A | G | 77 | K−>R |
| 316 | A | G | 84 | N−>D |
| 369 | G | A | 101 | M−>I |
| 395 | A | G | 110 | Q−>R |
| 465 | G | A | N/A | No change |

NOV26b SNP Data:

NOV26b has one SNP variant, whose variant position for its nucleotide and amino acid sequences is numbered according to SEQ ID NOs:111 and 112, respectively. The nucleotide sequence of the NOV26b variant differs as shown in Table 124.

TABLE 124
cSNP and Coding Variants for NOV26b

| | | | | Putative |
|---|---|---|---|---|
| NT Position | Wild Type | | | Allele |
| of cSNP | NT | Variant NT | Depth | Frequency |
| 133 | A | G | 41 | 0.049 |
| 268 | A | G | 41 | 0.049 |
| 324 | A | G | 41 | 0.049 |
| 372 | A | G | 41 | 0.049 |
| 376 | A | * | 41 | 0.049 |
| 456 | T | C | 40 | 0.050 |
| 488 | A | G | 32 | 0.344 |

NOV27a SNP Data:

NOV27a has one SNP variant, whose variant position for its nucleotide and amino acid sequences is numbered according to SEQ ID NOs: 113 and 114, respectively. The nucleotide sequence of the NOV27a variant differs as shown in Table 125.

TABLE 125
cSNP and Coding Variants for NOV27a

| NT Position | Wild Type | | Amino Acid | Amino Acid |
|---|---|---|---|---|
| of cSNP | NT | Variant NT | position | Change |
| 158 | A | T | 36 | No change |
| 491 | C | T | 147 | No change |
| 562 | T | C | 171 | L−>P |
| 858 | C | T | 270 | No change |
| 1750 | C | T | 567 | P−>L |

NOV27b SNP Data:

NOV27b has one SNP variant, whose variant position for its nucleotide and amino acid sequences is numbered according to SEQ ID NOs:115 and 116, respectively. The nucleotide sequence of the NOV27b variant differs as shown in Table 126.

TABLE 126
cSNP and Coding Variants for NOV27b

| | | | | Putative |
|---|---|---|---|---|
| NT Position | Wild Type | | | Allele |
| of cSNP | NT | Variant NT | Depth | Frequency |
| 131 | G | A | 43 | 0.233 |
| 1072 | G | A | 14 | 0.214 |
| 1368 | A | G | 24 | 0.125 |
| 1439 | G | A | 42 | 0.071 |
| 1733 | G | A | 43 | 0.047 |
| 1772 | T | A | 43 | 0.442 |
| 1787 | G | A | 42 | 0.286 |

NOV29c SNP Data:

NOV29c has one SNP variant, whose variant position for its nucleotide and amino acid sequences is numbered according to SEQ ID NOs:125 and 126, respectively. The nucleotide sequence of the NOV29c variant differs as shown in Table 127.

TABLE 127
cSNP and Coding Variants for NOV29c

| NT Position | Wild Type | | Amino Acid | Amino Acid |
|---|---|---|---|---|
| of cSNP | NT | Variant NT | position | Change |
| 760 | A | G | 254 | T−>A |
| 923 | T | C | 308 | G−>D |

NOV30 SNP Data:

NOV30 has one SNP variant, whose variant position for its nucleotide and amino acid sequences is numbered according to SEQ ID NOs:127 and 128, respectively. The nucleotide sequence of the NOV30 variant differs as shown in Table 128.

TABLE 128
cSNP and Coding Variants for NOV30

| NT Position | Wild Type | | Amino Acid | Amino Acid |
|---|---|---|---|---|
| of cSNP | NT | Variant NT | position | Change |
| 103 | A | G | 28 | I−>V |
| 207 | T | C | 62 | No change |
| 225 | C | T | 68 | No change |
| 233 | A | G | 71 | D−>G |
| 267 | T | C | 82 | No change |
| 318 | A | G | 99 | No change |
| 392 | T | C | 124 | L−>P |
| 431 | T | C | 137 | M−>T |
| 464 | A | G | 148 | E−>G |
| 479 | T | A | 153 | V−>E |

NOV33 SNP Data:

NOV33 has one SNP variant, whose variant position for its nucleotide and amino acid sequences is numbered according to SEQ ID NOs:133 and 134, respectively. The nucleotide sequence of the NOV33 variant differs as shown in Table 129.

TABLE 129
cSNP and Coding Variants for NOV33

| NT Position | Wild Type | | Amino Acid | Amino Acid |
|---|---|---|---|---|
| of cSNP | NT | Variant NT | position | Change |
| 5097 | T | C | 1699 | No change |
| 6012 | C | T | 2004 | No change |

NOV36a SNP Data:

NOV36a has one SNP variant, whose variant position for its nucleotide and amino acid sequence is numbered according to SEQ ID NOs:139 and 140, respectively. The nucleotide sequence of the NOV36a variant differs as shown in Table 130.

TABLE 130
cSNP and Coding Variants for NOV36a

| NT Position | Wild Type | | Amino Acid | Amino Acid |
|---|---|---|---|---|
| of cSNP | NT | Variant NT | position | Change |
| 351 | T | C | 102 | No change |
| 737 | A | G | 231 | D−>G |

NOV38 SNP Data:

NOV38 has one SNP variant, whose variant position for its nucleotide and amino acid sequence is numbered according to SEQ ID NOs:145 and 146, respectively. The nucleotide sequence of the NOV38 variant differs as shown in Table 131.

TABLE 131
cSNP and Coding Variants for NOV38

| NT Position | Wild Type | | Amino Acid | Amino Acid |
|---|---|---|---|---|
| of cSNP | NT | Variant NT | position | Change |
| 566 | C | T | 188 | H−>R |
| 658 | A | G | 219 | H−>R |
| 844 | G | A | 281 | C−>Y |
| 892 | C | T | 297 | A−>V |
| 910 | T | C | 303 | V−>A |
| 1009 | G | A | 336 | S−>N |

| | | | | Putative |
|---|---|---|---|---|
| NT Position | Wild Type | | | Allele |
| of cSNP | NT | Variant NT | Depth | Frequency |
| 95 | T | C | 14 | N/A |

NOV39a SNP Data:

NOV39a has one SNP variant, whose variant position for its nucleotide and amino acid sequence is numbered according to SEQ ID NOs:147 and 148, respectively. The nucleotide sequence of the NOV39a variant differs as shown in Table 132.

TABLE 132
cSNP and Coding Variants for NOV39a

| | | | | Putative |
|---|---|---|---|---|
| NT Position | Wild Type | | | Allele |
| of cSNP | NT | Variant NT | Depth | Frequency |
| 1095 | T | C | 11 | N/A |

NOV39b SNP Data:

NOV39b has one SNP variant, whose variant position for its nucleotide and amino acid sequences is numbered according to SEQ ID NOs:149 and 150, respectively. The nucleotide sequence of the NOV39b variant differs as shown in Table 133.

TABLE 133
cSNP and Coding Variants for NOV39b

| | | | | Putative |
|---|---|---|---|---|
| NT Position | Wild Type | | | Allele |
| of cSNP | NT | Variant NT | Depth | Frequency |
| 933 | C | T | 9 | 0.222 |

NOV42c SNP Data:

NOV42c has one SNP variant, whose variant position for its nucleotide and amino acid sequences is numbered according to SEQ ID NOs:161 and 162, respectively. The nucleotide sequence of the NOV42c variant differs as shown in Table 134.

TABLE 134
cSNP and Coding Variants for NOV42c

| NT Position | Wild Type | | Amino Acid | Amino Acid |
|---|---|---|---|---|
| of cSNP | NT | Variant NT | position | Change |
| 330 | G | A | 103 | R−>Q |
| 783 | A | G | 254 | D−>G |
| 903 | G | A | 294 | R−>H |
| 1389 | A | T | 456 | E−>V |
| 1389 | A | G | 456 | E−>G |
| 1394 | G | A | 458 | A−>T |
| 1642 | C | T | 540 | No change |
| 1656 | T | C | 545 | V−>A |
| 1658 | G | A | 546 | A−>T |

NOV43 SNP Data:

NOV43 has one SNP variant, whose variant position for its nucleotide and amino acid sequences is numbered according to SEQ ID NOs:165 and 166, respectively. The nucleotide sequence of the NOV43 variant differs as shown in Table 135.

TABLE 135
cSNP and Coding Variants for NOV43

| NT Position | Wild Type | | Amino Acid | Amino Acid |
|---|---|---|---|---|
| of cSNP | NT | Variant NT | position | Change |
| 378 | G | A | 121 | No change |
| 496 | G | A | 162 | R−>Q |

NOV46b SNP Data:

NOV46b has one SNP variant, whose variant position for its nucleotide and amino acid sequences is numbered according to SEQ ID NOs:173 and 174, respectively. The nucleotide sequence of the NOV46b variant differs as shown in Table 136.

TABLE 136
cSNP and Coding Variants for NOV46b

| NT Position | Wild Type | | Amino Acid | Amino Acid |
|---|---|---|---|---|
| of cSNP | NT | Variant NT | position | Change |
| 500 | C | T | 163 | No change |

| | | | | Putative |
|---|---|---|---|---|
| NT Position | Wild Type | | | Allele |
| of cSNP | NT | Variant NT | Depth | Frequency |
| 486 | C | T | 16 | 0.125 |

NOV48a SNP Data:

NOV48a has one SNP variant, whose variant position for its nucleotide and amino acid sequences is numbered according to SEQ ID NOs:181 and 182, respectively. The nucleotide sequence of the NOV48a variant differs as shown in Table 137.

TABLE 137
cSNP and Coding Variants for NOV48a

| NT Position | Wild Type | | Amino Acid | Amino Acid |
|---|---|---|---|---|
| of cSNP | NT | Variant NT | position | Change |
| 370 | A | G | 1 | M−>V |
| 436 | C | T | 23 | L−>F |
| 539 | A | G | 57 | D−>G |
| 650 | A | G | 94 | E−>G |
| 1012 | C | T | 215 | Q−>End |
| 1922 | A | G | 518 | K−>R |
| 2057 | A | G | 563 | Q−>R |
| 2066 | C | T | 566 | A−>V |
| 2198 | C | T | 610 | P−>L |
| 2618 | A | G | 750 | D−>G |
| 2656 | G | A | N/A | No change |

NOV50b SNP Data:

NOV50b has one SNP variant, whose variant position for its nucleotide and amino acid sequences is numbered according to SEQ ID NOs:189 and 190, respectively. The nucleotide sequence of the NOV50b variant differs as shown in Table 138.

TABLE 138
cSNP and Coding Variants for NOV50b

| NT Position | Wild Type | | Amino Acid | Amino Acid |
|---|---|---|---|---|
| of cSNP | NT | Variant NT | position | Change |
| 797 | A | G | 265 | Q−>R |

NOV52 SNP Data:

NOV52 has one SNP variant, whose variant position for its nucleotide and amino acid sequences is numbered according to SEQ ID NOs:193 and 194, respectively. The nucleotide sequence of the NOV52 variant differs as shown in Table 139.

TABLE 139
cSNP and Coding Variants for NOV52

| NT Position | Wild Type | | Amino Acid | Amino Acid |
|---|---|---|---|---|
| of cSNP | NT | Variant NT | position | Change |
| 318 | T | C | 48 | No change |
| 351 | C | T | 59 | R−>C |
| 1961 | G | T | 595 | M−>I |

| | | | | Putative |
|---|---|---|---|---|
| NT Position | Wild Type | | | Allele |
| of cSNP | NT | Variant NT | Depth | Frequency |
| 70 | C | T | 54 | 0.056 |

NOV56a SNP Data:

NOV56a has one SNP variant, whose variant position for its nucleotide and amino acid sequences is numbered according to SEQ ID NOs:203 and 204, respectively. The nucleotide sequence of the NOV56a variant differs as shown in Table 140.

TABLE 140
cSNP and Coding Variants for NOV56a

| NT Position | Wild Type | | Amino Acid | Amino Acid |
|---|---|---|---|---|
| of cSNP | NT | Variant NT | position | Change |
| 118 | A | C | 39 | No change |
| 435 | C | T | 135 | P−>L |
| 439 | T | C | 146 | No change |
| 473 | A | G | 158 | T−>A |
| 588 | T | C | 196 | V−>A |
| 596 | G | A | 199 | G−>R |
| 614 | A | G | 205 | M−>V |
| 631 | T | C | 210 | No change |
| 637 | A | G | 212 | No change |
| 642 | T | C | 214 | M−>T |
| 732 | G | T | 244 | W−>L |
| 902 | A | T | 301 | M−>L |

NOV57 SNP Data:

NOV57 has one SNP variant, whose variant position for its nucleotide and amino acid sequences is numbered according to SEQ ID NOs:207 and 208, respectively. The nucleotide sequence of the NOV57 variant differs as shown in Table 141.

TABLE 141
cSNP and Coding Variants for NOV57

| NT Position | Wild Type | | Amino Acid | Amino Acid |
|---|---|---|---|---|
| of cSNP | NT | Variant NT | position | Change |
| 939 | T | A | N/A | No change |

NOV58b SNP Data:

NOV58b has one SNP variant, whose variant position for its nucleotide and amino acid sequences is numbered according to SEQ ID NOs:211 and 212, respectively. The nucleotide sequence of the NOV58b variant differs as shown in Table 142.

TABLE 142
cSNP and Coding Variants for NOV58b

| | | | | Putative |
|---|---|---|---|---|
| NT Position | Wild Type | | | Allele |
| of cSNP | NT | Variant NT | Depth | Frequency |
| 88 | T | C | 11 | 0.273 |
| 377 | A | G | 18 | 0.111 |
| 500 | T | C | 18 | 0.111 |
| 509 | A | G | 18 | 0.111 |
| 570 | T | C | 17 | 0.118 |
| 647 | C | T | 9 | 0.222 |

NOV60a SNP Data:

NOV60a has one SNP variant, whose variant position for its nucleotide and amino acid sequences is numbered according to SEQ ID NOs:215 and 216, respectively. The nucleotide sequence of the NOV60a variant differs as shown in Table 143.

TABLE 143
cSNP and Coding Variants for NOV60a

| | | | | Putative |
|---|---|---|---|---|
| NT Position | Wild Type | | | Allele |
| of cSNP | NT | Variant NT | Depth | Frequency |
| 341 | T | C | 52 | |
| 401 | G | C | 79 | |
| 411 | T | C | 79 | |
| 444 | C | T | 79 | |
| 644 | C | T | 59 | |
| 653 | T | A | 59 | |
| 670 | T | C | 59 | |
| 707 | T | C | 33 | |

NOV60b SNP Data:

NOV60b has one SNP variant, whose variant position for its nucleotide and amino acid sequences is numbered according to SEQ ID NOs:217 and 218, respectively. The nucleotide sequence of the NOV60b variant differs as shown in Table 144.

TABLE 144
cSNP and Coding Variants for NOV60b

| | | | | Putative |
|---|---|---|---|---|
| NT Position | Wild Type | | | Allele |
| of cSNP | NT | Variant NT | Depth | Frequency |
| 162 | T | G | 54 | 0.259 |
| 192 | A | G | 54 | 0.056 |
| 229 | A | G | 81 | 0.025 |
| 246 | A | T | 80 | 0.025 |
| 255 | G | A | 79 | 0.038 |
| 263 | A | G | 77 | 0.039 |
| 342 | G | A | 103 | 0.019 |
| 389 | A | G | 105 | 0.019 |

Example 4. In-frame Cloning

NOV7c

For NOV7c the cDNA coding for the DOMAIN of NOV7c from residues 1 to 230 was targeted for “in-frame” cloning by PCR. The PCR template was based on the previously identified plasmid, when available, or on human cDNA(s).

TABLE 145
Oligonucleotide primers used to clone the target cDNA sequence:

| Primers | Sequences | |
|---|---|---|
| F1 | 5′-AGATCTCCCACC ATGCAACTTCAGGACCTGGAACTGC-3′ | (SEQ ID NO:1382) |
| R1 | 5′-CTCGAG TCCACTTACAATTTCCCGTCTCATTTCC-3′ | (SEQ ID NO:1385) |
| SF1 | 5′-TCCTCCTGGAGAAAGCTCAGAATCTGTTTT-3′ | (SEQ ID NO:1387) |
| SF2 | 5′-CTCCAGATTTGGAAAGTTCTGAGGAA-3′ | (SEQ ID NO:1388) |
| SR1 | 5′-ATTTCTCCAAGTCCCAGGCCC-3′ | (SEQ ID NO:1389) |
| SR2 | 5′-GAGCCTGTTCTAGAAGGAGCTGTTG-3′ | (SEQ ID NO:1390) |

For downstream cloning purposes, the forward primer includes an in-frame Hind III restriction site and the reverse primer contains an in-frame Xho I restriction site.

Two parallel PCR reactions were set up using a total of 0.5-1.0 ng human pooled cDNA as template for each reaction. The pool is composed of 5 micrograms of each of the following human tissue cDNAs: adrenal gland, whole brain, amygdala, cerebellum, thalamus, bone marrow, fetal brain, fetal kidney, fetal liver, fetal lung, heart, kidney, liver, lymphoma, Burkitt's Raji cell line, mammary gland, pancreas, pituitary gland, placenta, prostate, salivary gland, skeletal muscle, small Intestine, spleen, stomach, thyroid, trachea, uterus.

When the tissue of expression is known and available, the second PCR was performed using the above primers and 0.5 ng-1.0 ng of one of the following human tissue cDNAs: skeleton muscle, testis, mammary gland, adrenal gland, ovary, colon, normal cerebellum, normal adipose, normal skin, bone marrow, brain amygdala, brain hippocampus, brain substantia nigra, brain thalamus, thyroid, fetal lung, fetal liver, fetal brain, kidney, heart, spleen, uterus, pituitary gland, lymph node, salivary gland, small intestine, prostate, placenta, spinal cord, peripheral blood, trachea, stomach, pancreas, hypothalamus.

The reaction mixtures contained 2 microliters of each of the primers (original concentration: 5 pmol/ul), 1 microliter of 10 mM dNTP (Clontech Laboratories, Palo Alto Calif.) and 1 microliter of 50× Advantage-HF 2 polymerase (Clontech Laboratories) in 50 microliter-reaction volume. The following reaction conditions were used:

PCR condition 1:

| | | |
|---|---|---|
| a) | 96° C. | 3 minutes |
| b) | 96° C. | 30 seconds denaturation |
| c) | 60° C. | 30 seconds, primer annealing |
| d) | 72° C. | 6 minutes extension |

Repeat steps b-d 15 times

| | | |
|---|---|---|
| e) | 96° C. | 15 seconds denaturation |
| f) | 60° C. | 30 seconds, primer annealing |
| g) | 72° C. | 6 minutes extension |

Repeat steps e-g 29 times

| | | |
|---|---|---|
| e) | 72° C. | 10 minutes final extension |

PCR condition 2:

| | | |
|---|---|---|
| a) | 96° C. | 3 minutes |
| b) | 96° C. | 15 seconds denaturation |
| c) | 76° C. | 30 seconds, primer annealing, reducing the temperature |
| | | by 1° C. per cycle |
| d) | 72° C. | 4 minutes extension |

Repeat steps b-d 34 times

| | | |
|---|---|---|
| e) | 72° C. | 10 minutes final extension |

Example 5: SAGE Analysis

Hs 181638: ESTs, Weakly similar to SSR1_HUMAN SOMATOSTATIN RECEPTOR TYPE 1 [H.sapiens] SAGE library data and reliable tag summary reliable tags found in SAGE libraries:

| | | Tags | | |
|---|---|---|---|---|
| | | per | Tag | Total |
| TTGTCCATAT | Library name | million | counts | tags |
| | SAGE Chen LNCaP | 16 | 1 | 62267 |
| | SAGE Chen Normal Pr | 30 | 2 | 66193 |
| | SAGE Chen Tumor Pr | 14 | 1 | 68334 |
| | SAGE CAPAN1 | 26 | 1 | 37926 |
| | SAGE Panc1 | 80 | 2 | 24879 |
| | SAGE Duke H54 | 34 | 2 | 57164 |
| | EGFRvIII | | | |
| | SAGE CPDR LNCaP-T | 22 | 1 | 44122 |
| | SAGE 293-IND | 40 | 1 | 24481 |
| | SAGE PR317 | 16 | 1 | 59419 |
| | normal prostate | | | |
| | SAGE PR317 | 15 | 1 | 65109 |
| | prostate tumor | | | |
| | SAGE BB542 | 10 | 1 | 94806 |
| | whitematter | | | |
| | SAGE Panc 96-6252 | 27 | 1 | 35745 |
| | SAGE SciencePark | 16 | 1 | 61079 |
| | MCF7 | | | |
| | Control 0 h | | | |
| | SAGE SciencePark | 16 | 1 | 60435 |
| | MCF7 | | | |
| | estradiol 10 h | | | |
| | SAGE Duke H566 | 15 | 1 | 65728 |
| | SAGE OVT-6 | 23 | 1 | 42336 |
| | SAGE mammary | 20 | 1 | 49167 |
| | epithelium | | | |
| | SAGE ML10-10 | 35 | 2 | 56943 |
| | SAGE Duke H1043 | 13 | 1 | 76673 |

### Other Embodiments

Although particular embodiments have been disclosed herein in detail, this has been done by way of example for purposes of illustration only, and is not intended to be limiting with respect to the scope of the appended claims, which follow. In particular, it is contemplated by the inventors that various substitutions, alterations, and modifications may be made to the invention without departing from the spirit and scope of the invention as defined by the claims. The choice of nucleic acid starting material, clone of interest, or library type is believed to be a matter of routine for a person of ordinary skill in the art with knowledge of the embodiments described herein. Other aspects, advantages, and modifications considered to be within the scope of the following claims.

What is claimed is:

1. An isolated polypeptide comprising an amino acid sequence selected from the group consisting of:
(a) a mature form of an amino acid sequence selected from the group consisting of SEQ ID NOS:2n, wherein n is an integer between 1 and 162;
(b) a variant of a mature form of an amino acid sequence selected from the group consisting of SEQ ID NOS:2n, wherein n is an integer between 1 and 162, wherein one or more amino acid residues in said variant differs from the amino acid sequence of said mature form, provided that said variant differs in no more than 15% of the amino acid residues from the amino acid sequence of said mature form;
(c) an amino acid sequence selected from the group consisting SEQ ID NOS:2n, wherein n is an integer between 1 and 162; and
(d) a variant of an amino acid sequence selected from the group consisting of SEQ ID NOS:2n, wherein n is an integer between 1 and 162, wherein one or more amino acid residues in said variant differs from the amino acid sequence of said nature form, provided that said variant differs in no more than 15% of amino acid residues from said amino acid sequence.

2. The polypeptide of claim 1, wherein said polypeptide comprises the amino acid sequence of a naturally-occurring allelic variant of an amino acid sequence selected from the group consisting SEQ ID NOS:2n, wherein n is an integer between 1 and 162.

3. The polypeptide of claim 2, wherein said allelic variant comprises an amino acid sequence that is the translation of a nucleic acid sequence differing by a single nucleotide from a nucleic acid sequence selected from the group consisting of SEQ ID NOS:2n−1, wherein n is an integer between 1 and 162.

4. The polypeptide of claim 1, wherein the amino acid sequence of said variant comprises a conservative amino acid substitution.

5. An isolated nucleic acid molecule comprising a nucleic acid sequence encoding a polypeptide comprising an amino acid sequence selected from the group consisting of:
(a) a mature form of an amino acid sequence selected from the group consisting of SEQ ID NOS:2n, wherein n is an integer between 1 and 162;
(b) a mature form of an amino acid sequence selected from the group consisting of SEQ ID NOS:2n, wherein n is an integer between 1 and 162, wherein one or more amino acid residues in said variant differs from the amino acid sequence of said mature form, provided that said variant differs in no more than 15% of the amino acid residues from the amino acid sequence of said mature form;
(c) an amino acid sequence selected from the group consisting of SEQ ID NOS:2n, wherein n is an integer between 1 and 162;
(d) a variant of an amino acid sequence selected from the group consisting SEQ ID NOS:2n, wherein n is an integer between 1 and 162, wherein one or more amino acid residues in said variant differs from the amino acid sequence of said mature form, provided that said variant differs in no more than 15% of amino acid residues from said amino acid sequence;
(e) a nucleic acid fragment encoding at least a portion of a polypeptide comprising an amino acid sequence chosen from the group consisting of SEQ ID NOS:2n, wherein n is an integer between 1 and 162, or a variant of said polypeptide, wherein one or more amino acid residues in said variant differs from the amino acid sequence of said mature form, provided that said variant differs in no more than 15% of amino acid residues from said amino acid sequence; and
(f) a nucleic acid molecule comprising the complement of (a), (b), (c), (d) or (e).

6. The nucleic acid molecule of claim 5, wherein the nucleic acid molecule comprises the nucleotide sequence of a naturally-occurring allelic nucleic acid variant.

7. The nucleic acid molecule of claim 5, wherein the nucleic acid molecule encodes a polypeptide comprising the amino acid sequence of a naturally-occurring polypeptide variant.

8. The nucleic acid molecule of claim 5, wherein the nucleic acid molecule differs by a single nucleotide from a nucleic acid sequence selected from the group consisting of SEQ ID NOS:2n−1, wherein n is an integer between 1 and 162.

9. The nucleic acid molecule of claim 5, wherein said nucleic acid molecule comprises a nucleotide sequence selected from the group consisting of:
(a) a nucleotide sequence selected from the group consisting of SEQ ID NOS:2n−1, wherein n is an integer between 1 and 162;
(b) a nucleotide sequence differing by one or more nucleotides from a nucleotide sequence selected from the group consisting of SEQ ID NOS:2n−1, wherein n is an integer between 1 and 162, provided that no more than 20% of the nucleotides differ from said nucleotide sequence;
(c) a nucleic acid fragment of (a); and
(d) a nucleic acid fragment of (b).

10. The nucleic acid molecule of claim 5, wherein said nucleic acid molecule hybridizes under stringent conditions to a nucleotide sequence chosen from the group consisting SEQ ID NOS:2n−1, wherein n is an integer between 1 and 162, or a complement of said nucleotide sequence.

11. The nucleic acid molecule of claim 5, wherein the nucleic acid molecule comprises a nucleotide sequence selected from the group consisting of:
(a) a first nucleotide sequence comprising a coding sequence differing by one or more nucleotide sequences from a coding sequence encoding said amino acid sequence, provided that no more than 20% of the nucleotides in the coding sequence in said first nucleotide sequence differ from said coding sequence;
(b) an isolated second polynucleotide that is a complement of the first polynucleotide; and
(c) a nucleic acid fragment of (a) or (b).

12. A vector comprising the nucleic acid molecule of claim 11.

13. The vector of claim 12, further comprising a promoter operably-linked to said nucleic acid molecule.

14. A cell comprising the vector of claim 12.

15. An antibody that binds immunospecifically to the polypeptide of claim 1.

16. The antibody of claim 15, wherein said antibody is a monoclonal antibody.

17. The antibody of claim 15, wherein the antibody is a humanized antibody.

18. A method for determining the presence or amount of the polypeptide of claim 1 in a sample, the method comprising:
(a) providing the sample;
(b) contacting the sample with an antibody that binds immunospecifically to the polypeptide; and
(c) determining the presence or amount of antibody bound to said polypeptide,
thereby determining the presence or amount of polypeptide in said sample.

19. A method for determining the presence or amount of the nucleic acid molecule of claim 5 in a sample, the method comprising:
(a) providing the sample;
(b) contacting the sample with a probe that binds to said nucleic acid molecule; and
(c) determining the presence or amount of the probe bound to said nucleic acid molecule,
thereby determining the presence or amount of the nucleic acid molecule in said sample.

20. The method of claim 19 wherein presence or amount of the nucleic acid molecule is used as a marker for cell or tissue type.

21. The method of claim 20 wherein the cell or tissue type is cancerous.

22. A method of identifying an agent that binds to a polypeptide of claim 1, the method comprising:
(a) contacting said polypeptide with said agent; and
(b) determining whether said agent binds to said polypeptide.

23. The method of claim 22 wherein the agent is a cellular receptor or a downstream effector.

24. A method for identifying an agent that modulates the expression or activity of the polypeptide of claim 1, the method comprising:
(a) providing a cell expressing said polypeptide;
(b) contacting the cell with said agent, and
(c) determining whether the agent modulates expression or activity of said polypeptide,
whereby an alteration in expression or activity of said peptide indicates said agent modulates expression or activity of said polypeptide.

25. A method for modulating the activity of the polypeptide of claim 1, the method comprising contacting a cell sample expressing the polypeptide of said claim with a compound that binds to said polypeptide in an amount sufficient to modulate the activity of the polypeptide.

26. A method of treating or preventing a NOVX-associated disorder, said method comprising administering to a subject in which such treatment or prevention is desired the polypeptide of claim 1 in an amount sufficient to treat or prevent said NOVX-associated disorder in said subject.

27. The method of claim 26 wherein the disorder is selected from the group consisting of cardiomyopathy and atherosclerosis.

28. The method of claim 26 wherein the disorder is related to cell signal processing and metabolic pathway modulation.

29. The method of claim 26, wherein said subject is a human.

30. A method of treating or preventing a NOVX-associated disorder, said method comprising administering to a subject in which such treatment or prevention is desired the nucleic acid of claim 5 in an amount sufficient to treat or prevent said NOVX-associated disorder in said subject.

31. The method of claim 30 wherein the disorder is selected from the group consisting of cardiomyopathy and atherosclerosis.

32. The method of claim 30 wherein the disorder is related to cell signal processing and metabolic pathway modulation.

33. The method of claim 30, wherein said subject is a human.

34. A method of treating or preventing a NOVX-associated disorder, said method comprising administering to a subject in which such treatment or prevention is desired the antibody of claim 15 in an amount sufficient to treat or prevent said NOVX-associated disorder in said subject.

35. The method of claim 34 wherein the disorder is diabetes.

36. The method of claim 34 wherein the disorder is related to cell signal processing and metabolic pathway modulation.

37. The method of claim 34, wherein the subject is a human.

38. A pharmaceutical composition comprising the polypeptide of claim 1 and a pharmaceutically-acceptable carrier.

39. A pharmaceutical composition comprising the nucleic acid molecule of claim 5 and a pharmaceutically-acceptable carrier.

40. A pharmaceutical composition comprising the antibody of claim 15 and a pharmaceutically-acceptable carrier.

41. A kit comprising in one or more containers, the pharmaceutical composition of claim 38.

42. A kit comprising in one or more containers, the pharmaceutical composition of claim 39.

43. A kit comprising in one or more containers, the pharmaceutical composition of claim 40.

44. A method for determining the presence of or predisposition to a disease associated with altered levels of the polypeptide of claim 1 in a first mammalian subject, the method comprising:
(a) measuring the level of expression of the polypeptide in a sample from the first mammalian subject; and
(b) comparing the amount of said polypeptide in the sample of step (a) to the amount of the polypeptide present in a control sample from a second mammalian subject known not to have, or not to be predisposed to, said disease;
wherein an alteration in the expression level of the polypeptide in the first subject as compared to the control sample indicates the presence of or predisposition to said disease.

45. The method of claim 44 wherein the predisposition is to a cancer.

46. A method for determining the presence of or predisposition to a disease associated with altered levels of the nucleic acid molecule of claim 5 in a first mammalian subject, the method comprising:
(a) measuring the amount of the nucleic acid in a sample from the first mammalian subject; and
(b) comparing the amount of said nucleic acid in the sample of step (a) to the amount of the nucleic acid present in a control sample from a second mammalian subject known not to have or not be predisposed to, the disease;
wherein an alteration in the level of the nucleic acid in the first subject as compared to the control sample indicates the presence of or predisposition to the disease.

47. The method of claim 46 wherein the predisposition is to a cancer.

48. A method of treating a pathological state in a mammal, the method comprising administering to the mammal a polypeptide in an amount that is sufficient to alleviate the pathological state, wherein the polypeptide is a polypeptide having an amino acid sequence at least 95% identical to a polypeptide comprising an amino acid sequence of at least one SEQ ID NOS:2n, wherein n is an integer between 1 and 162, or a biologically active fragment thereof.

49. A method of treating a pathological state in a mammal, the method comprising administering to the mammal the antibody of claim 15 in an amount sufficient to alleviate the pathological state.
